(19) United States
(12) Patent Application Publication
(10) Pub. No.: US 2024/0150377 A1
(43) Pub. Date: May 9, 2024

(54) MODULATORS OF CYSTIC FIBROSIS TRANSMEMBRANE CONDUCTANCE REGULATOR

(71) Applicant: VERTEX PHARMACEUTICALS INCORPORATED, Boston, MA (US)

(72) Inventors: Jason MCCARTNEY, Cardiff by the Sea, CA (US); Alexander Russell ABELA, Escondido, CA (US); Sunny ABRAHAM, San Diego, CA (US); Corey Don ANDERSON, Brighton, MA (US); Vijayalaksmi ARUMUGAM, San Marcos, CA (US); Jaclyn CHAU, San Diego, CA (US); Jeremy CLEMENS, San Diego, CA (US); Thomas CLEVELAND, San Marcos, CA (US); Timothy Richard COON, Carlsbad, CA (US); Timothy A. DWIGHT, Quincy, MA (US); Lev Tyler Dewey FANNING, San Marcos, CA (US); Bryan A. FRIEMAN, La Jolla, CA (US); Peter GROOTENHUIS, Del Mar, CA (US); Anton V. GULEVICH, San Diego, CA (US); Sara Sabina HADIDA RUAH, La Jolla, CA (US); Yoshihiro ISHIHARA, San Diego, CA (US); Haripada KHATUYA, San Diego, CA (US); Paul KRENITSKY, San Francisco, CA (US); Vito MELILLO, Escondido, CA (US); Mark Thomas MILLER, Rancho Santa Fe, CA (US); Prasuna PARASELLI, San Diego, CA (US); Fabrice PIERRE, La Jolla, CA (US); Alina SILINA, Needham, MA (US); Joe A. TRAN, San Marcos, CA (US); Johnny UY, San Diego, CA (US); Lino VALDEZ, San Diego, CA (US); Troy VICKERS, San Diego, CA (US); Jinglan ZHOU, San Diego, CA (US)

(21) Appl. No.: 18/248,071

(22) PCT Filed: Oct. 6, 2021
(86) PCT No.: PCT/US2021/053858

§ 371 Date: Apr. 6, 2023

Related U.S. Application Data
(60) Provisional application No. 63/088,686, filed on Oct. 7, 2020.

(51) Int. Cl. C07D 515/08; A61K 31/404; A61K 31/47; A61K 31/529

(52) U.S. Cl. CPC C07D 515/08; A61K 31/404; A61K 31/47; A61K 31/529

(57) ABSTRACT

This disclosure provides modulators of Cystic Fibrosis Transmembrane Conductance Regulator (CFTR) having core structure (I), pharmaceutical compositions containing at least one such modulator, methods of treatment of CFTR mediated diseases, including cystic fibrosis, using such modulators and pharmaceutical compositions, combination pharmaceutical compositions and combination therapies employing those modulators, and processes and intermediates for making such modulators.

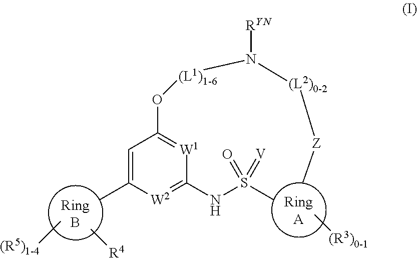

This application claims the benefit of priority of U.S. Provisional Application No. 63/088,686, filed Oct. 7, 2020, the contents of which are incorporated by reference herein in their entirety.

The disclosure relates to modulators of Cystic Fibrosis Transmembrane Conductance Regulator (CFTR), pharmaceutical compositions containing the modulators, methods of treatment of CFTR mediated diseases, including cystic fibrosis, using such modulators, combination therapies and combination pharmaceutical compositions employing such modulators, and processes and intermediates for making such modulators.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion causing an imbalance in ion and fluid transport. The resulting decrease in anion transport contributes to increased mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al. (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 mutations in the CF gene have been identified; currently, the CFTR2 database contains information on only 432 of these identified mutations, with sufficient evidence to define 352 mutations as disease causing. The most prevalent disease-causing mutation is a deletion of phenylalanine at position 508 of the CFTR amino acid sequence and is commonly referred to as the F508del mutation. This mutation occurs in many of the cases of cystic fibrosis and is associated with severe disease.

The deletion of residue 508 in CFTR prevents the nascent protein from folding correctly. This results in the inability of the mutant protein to exit the endoplasmic reticulum (ER) and traffic to the plasma membrane. As a result, the number of CFTR channels for anion transport present in the membrane is far less than observed in cells expressing wild-type CFTR, i.e., CFTR having no mutations. In addition to impaired trafficking, the mutation results in defective channel gating. Together, the reduced number of channels in the membrane and the defective gating lead to reduced anion and fluid transport across epithelia. (Quinton, P. M. (1990), FASEB J. 4: 2709-2727). The channels that are defective because of the F508del mutation are still functional, albeit less functional than wild-type CFTR channels. (Dalemans et al. (1991), Nature Lond. 354: 526-528; Pasyk and Foskett (1995), J. Cell. Biochem. 270: 12347-50). In addition to F508del, other disease-causing mutations in CFTR that result in defective trafficking, synthesis, and/or channel gating could be up- or down-regulated to alter anion secretion and modify disease progression and/or severity.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride transport takes place by the coordinated activity of ENaC and CFTR present on the apical membrane and the Na⁺—K⁺-ATPase pump and Cl⁻ channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Na⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

A number of CFTR modulating compounds have recently been identified. However, compounds that can treat or reduce the severity of cystic fibrosis and other CFTR mediated diseases, and particularly the more severe forms of these diseases, are still needed.

One aspect of the disclosure provides novel compounds, including compounds of Formula I, compounds of any of Formulae Ia, IIa, IIb, III, IV, V, and VI, Compounds 1-1193, Compounds 1194-1294, tautomers thereof, deuterated derivatives of those compounds and tautomers, and pharmaceutically acceptable salts of any of the foregoing.

Formula I encompasses compounds falling within the following structure:

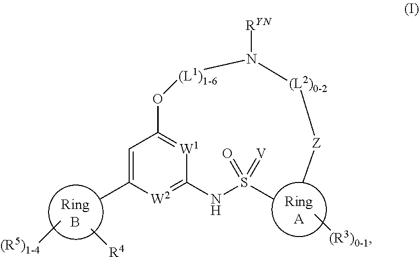

and includes tautomers thereof, deuterated derivatives of those compounds and tautomers, and pharmaceutically acceptable salts of any of the foregoing, wherein:
Ring A is selected from:
   C₆-C₁₀ aryl,
   C₃-C₁₀ cycloalkyl,
   3- to 10-membered heterocyclyl, and
   5- to 10-membered heteroaryl;
Ring B is selected from:
   C₆-C₁₀ aryl,
   C₃-C₁₀ cycloalkyl,
   3- to 10-membered heterocyclyl, and
   5- to 10-membered heteroaryl;
   V is selected from O and NH;
W¹ is selected from N and CH;
W² is selected from N and CH, provided that at least one of W¹ and W² is N;
Z is selected from O, NR^{ZN}, and C(R^{ZC})₂, provided that when L² is absent, Z is C(R^{ZC})₂.
each L¹ is independently selected from C(R^{L1})₂ and

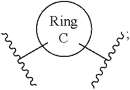

each L² is independently selected from C(R^{L2})₂;
Ring C is selected from C₆-C₁₀ aryl optionally substituted with 1-3 groups independently selected from:
   halogen,
   C₁-C₆ alkyl, and
   N(RN)₂;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl; each R⁵ is independently selected from:
   hydrogen,
   halogen,
   hydroxyl,
   N(R^{N})₂,
   —SO-Me,
   CH═C(R^{LC})₂, wherein both R^{LC} are taken together to form a C₃-C₁₀ cycloalkyl,
   C₁-C₆ alkyl optionally substituted with 1-3 groups independently selected from:
      hydroxyl,
      C₁-C₆ alkoxy optionally substituted with 1-3 groups independently selected from C₁-C₆ alkoxy and C₆-C₁₀ aryl,
      C₃-C₁₀ cycloalkyl,
      —(O)₀₋₁—(C₆-C₁₀ aryl) optionally substituted with 1-3 groups independently selected from C₁-C₆ alkyl and C₁-C₆ alkoxy,
      3- to 10-membered heterocyclyl, and
      N(R^{N})₂,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
R^{YN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy,
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl optionally substituted with 1-3 groups independently selected from oxo and C₁-C₆ alkoxy,
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl,
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3 —SiMe₃), 3- to 10-membered heterocyclyl, and N(R^{N})₂,
         C₁-C₆ alkoxy optionally substituted with 1-3 groups independently selected from hydroxyl, oxo, halogen, C₁-C₆ alkoxy, N(R^{N})₂, 3- to 10-membered heterocyclyl (optionally substituted with 1-3 groups independently selected from C₁-C₆ alkyl), and C₃-C₁₀ cycloalkyl,
         C₁-C₆ fluoroalkyl,
         —(O)₀₋₁—(C₃-C₁₀ cycloalkyl) optionally substituted with 1-3 groups independently selected from halogen, C₁-C₆ alkyl, and C₁-C₆ alkoxy,
         —(O)₀₋₁—(C₆-C₁₀ aryl),
         —(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, C₃-C₁₀ cycloalkyl, 5- to 10-membered heteroaryl (optionally substituted with 1-3 groups independently selected from C₁-C₆ alkyl), and 3- to 10-membered heterocyclyl (optionally substituted with 1-3 groups independently selected from C₁-C₆ fluoroalkyl), and
         5- to 10-membered heteroaryl optionally substituted with 1-4 groups independently selected from C₁-C₆ alkyl and C₃-C₁₀ cycloalkyl, and
   C₁-C₆ fluoroalkyl;
   R^{ZN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy,
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl optionally substituted with 1-3 groups independently selected from oxo and C₁-C₆ alkoxy,
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl,
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3 —SiMe₃), and N(R^{N})₂,
         C₁-C₆ alkoxy optionally substituted with 1-3 groups independently selected from hydroxyl, oxo, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl), and
         5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
         hydroxyl,
         oxo,
         N(R^{N})₂,
         C₁-C₆ alkoxy, and
         C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-member heterocyclyl optionally substituted with 1-3 groups independently selected from C₁-C₆ alkyl,
      5- to 10-membered heteroaryl optionally substituted with 1-3 groups independently selected from:
         hydroxyl,
         cyano,
         oxo,
         halogen,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl,
         3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from halogen and C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
   R^{F};
   each R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl),
   C₆-C₁₀ aryl optionally substituted with 1-3 groups independently selected from C₁-C₆ alkyl, and
   R^{F};
   or two R^{ZC} are taken together to form an oxo group;
   each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen, C₁-C₆ alkyl, and C₁-C₆ fluoroalkyl,
      —O—(C₃-C₁₀ cycloalkyl),
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl optionally substituted with 1-3 groups independently selected from C₁-C₆ alkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
         hydroxyl,
         oxo,
         cyano,
         SiMe₃,
         N(R^{N})₂, and
         C₃-C₁₀ cycloalkyl optionally substituted with 1-3 groups independently selected from C₁-C₆ fluoroalkyl,
      C₁-C₆ alkoxy optionally substituted with 1-3 groups independently selected from:
         C₃-C₁₀ cycloalkyl optionally substituted with 1-3 groups independently selected from C₁-C₆ fluoroalkyl, and
         C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
         oxo, and
         C₁-C₆ alkoxy,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl, and
   R^{F}.
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
      NHCOMe,
      N(R^{N3})₂, wherein each R^{N3} is independently selected from C₁-C₆ alkyl (optionally substituted with 1-3 groups independently selected from oxo and C₁-C₆ alkoxy),
      C₁-C₆ alkoxy optionally substituted with 1-3 groups independently selected from C₆-C₁₀ aryl, oxo, NMe₂, and NHMe,
      —(O)₀₋₁—(C₃-C₁₀ cycloalkyl) optionally substituted with 1-3 groups independently selected from C₁-C₆ alkyl (optionally substituted with 1-3 groups independently selected from C₁-C₆ alkoxy), and C₁-C₆ alkoxy,
      C₆-C₁₀ aryl optionally substituted with 1-3 groups independently selected from halogen and C₁-C₆ alkyl,
      3- to 14-membered heterocyclyl optionally substituted with 1-4 groups independently selected from oxo and C₁-C₆ alkyl, and
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      halogen,
      NH₂,
      NHMe,
      C₁-C₆ alkoxy, and
      C₁-C₆ alkyl optionally substituted with 1-3 groups independently selected from hydroxyl and C₁-C₆ alkoxy,
   C₆-C₁₀ aryl,
   5- to 10-membered heteroaryl optionally substituted with 1-3 groups independently selected from C₁-C₆ alkyl, and
   3- to 10-membered heterocyclyl;
or two R^{N} on the same nitrogen atom are taken together with the nitrogen to which they are bonded to form a 3- to 10-membered heterocyclyl optionally substituted with 1-4 groups selected from:
   hydroxyl,
   halogen,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(R^{N})₂,
      C₁-C₉ alkyl optionally substituted with 1-4 groups independently selected from:
         oxo,
         halogen,
         hydroxyl,
         N(R^{N})₂,
         —SO₂-(C₁-C₆ alkyl),
         C₁-C₆ alkoxy optionally substituted with 1-3 groups independently selected from halogen and C₆-C₁₀ aryl,
         C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
         —O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₃-C₁₂ cycloalkyl optionally substituted with 1-4 groups independently selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy and C₁-C₆ fluoroalkyl, and
   5- to 12-membered heteroaryl optionally substituted with 1-3 groups independently selected from C₁-C₆ alkyl and C₁-C₆ fluoroalkyl.

Formula I encompasses compounds of Formula Ia falling within the following structure:

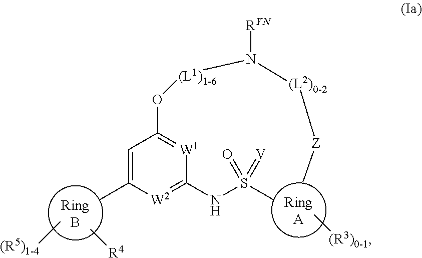

and includes tautomers thereof, deuterated derivatives of those compounds and tautomers, and pharmaceutically acceptable salts of any of the foregoing, wherein:
Ring A is selected from:
   C₆-C₁₀ aryl,
   C₃-C₁₀ cycloalkyl,
   3- to 10-membered heterocyclyl, and
   5- to 10-membered heteroaryl;
Ring B is selected from:
   C₆-C₁₀ aryl,
   C₃-C₁₀ cycloalkyl,
   3- to 10-membered heterocyclyl, and
   5- to 10-membered heteroaryl;
V is selected from O and NH;
W¹ is selected from N and CH;
W² is selected from N and CH, provided that at least one of W¹ and W² is N;
Z is selected from O, NR^{ZN}, and C(R^{ZC})₂, provided that when L² is absent, Z is C(R^{ZC})₂
each L¹ is independently selected from C(R^{L1})₂ and

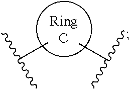

each L² is independently selected from C(R^{L2})₂;
Ring C is selected from C₆-C₁₀ aryl optionally substituted with 1-3 groups independently selected from:
   halogen,
   C₁-C₆ alkyl, and
   N(R^{N})₂;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl; each R⁵ is independently selected from:
   hydrogen,
   halogen,
   hydroxyl,
   N(R^{N})₂,
   —SO-Me,
   —CH═C(R^{LC})₂, wherein both R^{LC} are taken together to form a C₃-C₁₀ cycloalkyl,
   C₁-C₆ alkyl optionally substituted with 1-3 groups independently selected from:
      hydroxyl,
      C₁-C₆ alkoxy optionally substituted with 1-3 groups independently selected from C₁-C₆ alkoxy and C₆-C₁₀ aryl,
      C₃-C₁₀ cycloalkyl,
      —(O)₀₋₁—(C₆-C₁₀ aryl) optionally substituted with 1-3 groups independently selected from C₁-C₆ alkyl and C₁-C₆ alkoxy,
      3- to 10-membered heterocyclyl, and
      N(R^{N})₂,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
R^{YN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy,
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl optionally substituted with 1-3 groups independently selected from oxo and C₁-C₆ alkoxy,
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl,
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3 —SiMe₃), and N(R^{N})₂,
         C₁-C₆ alkoxy optionally substituted with 1-3 groups independently selected from hydroxyl, oxo, halogen, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from halogen, C₁-C₆ alkyl, and C₁-C₆ alkoxy,
         —(O)₀₋₁—(C₆-C₁₀ aryl),
         —(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, and 3- to 10-membered heterocyclyl (optionally substituted with 1-3 groups independently selected from C₁-C₆ fluoroalkyl), and
         5- to 10-membered heteroaryl optionally substituted with 1-4 groups independently selected from C₁-C₆ alkyl and C₃-C₁₀ cycloalkyl, and
   C₁-C₆ fluoroalkyl;
R^{ZN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy,
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl optionally substituted with 1-3 groups independently selected from oxo and C₁-C₆ alkoxy,
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl,
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3 —SiMe₃), and N(R^{N})₂,
         C₁-C₆ alkoxy optionally substituted with 1-3 groups independently selected from hydroxyl, oxo, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl), and
         5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
         hydroxyl,
         oxo,
         N(R^{N})₂,
         C₁-C₆ alkoxy, and
         C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-member heterocyclyl optionally substituted with 1-3 groups independently selected from C₁-C₆ alkyl,
      5- to 10-membered heteroaryl optionally substituted with 1-3 groups independently selected from:
         hydroxyl,
         cyano,
         oxo,
         halogen,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl,
         3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from halogen and C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
   R^{F};
each R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl),
   C₆-C₁₀ aryl optionally substituted with 1-3 groups independently selected from C₁-C₆ alkyl, and
   R^{F};
or two R^{ZC} are taken together to form an oxo group;
each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
         hydroxyl,
         oxo,
         cyano,
         SiMe₃,
         N(R^{N})₂, and
         C₃-C₁₀ cycloalkyl optionally substituted with 1-3 groups independently selected from C₁-C₆ fluoroalkyl,
      C₁-C₆ alkoxy optionally substituted with 1-3 groups independently selected from:
         C₃-C₁₀ cycloalkyl optionally substituted with 1-3 groups independently selected from C₁-C₆ fluoroalkyl, and
         C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
         oxo, and
         C₁-C₆ alkoxy,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl, and
   R^{F}.
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
      NHCOMe,
      C₁-C₆ alkoxy optionally substituted with 1-3 groups independently selected from C₆-C₁₀ aryl,
      —(O)₀₋₁—(C₃-C₁₀ cycloalkyl),
      C₆-C₁₀ aryl optionally substituted with 1-3 groups independently selected from halogen and C₁-C₆ alkyl,
      3- to 14-membered heterocyclyl optionally substituted with 1-4 groups independently selected from oxo and C₁-C₆ alkyl, and
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      NH₂,
      NHMe, and
      C₁-C₆ alkyl optionally substituted with 1-3 groups independently selected from hydroxyl,
   C₆-C₁₀ aryl, and
   3- to 10-membered heterocyclyl;
or two R^{N} on the same nitrogen atom are taken together with the nitrogen to which they are bonded to form a 3- to 10-membered heterocyclyl optionally substituted with 1-4 groups selected from:
   hydroxyl,
   halogen,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(R^{N})₂,
      C₁-C₉ alkyl optionally substituted with 1-4 groups independently selected from:
         oxo,
         halogen,
         hydroxyl,
         N(R^{N})₂,
         —SO₂-(C₁-C₆ alkyl),
         C₁-C₆ alkoxy optionally substituted with 1-3 groups independently selected from halogen and C₆-C₁₀ aryl,
         C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
         —O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₃-C₁₂ cycloalkyl optionally substituted with 1-4 groups independently selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy and C₁-C₆ fluoroalkyl, and
   5- to 12-membered heteroaryl optionally substituted with 1-3 groups independently selected from C₁-C₆ alkyl and C₁-C₆ fluoroalkyl.

Formula I also includes compounds of Formula Ib:

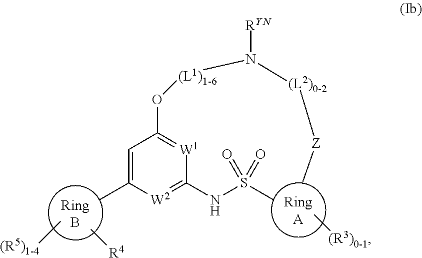

tautomers of those compounds, deuterated derivatives of any of the compounds and tautomers, and pharmaceutically acceptable salts of any of the foregoing, wherein all variables are as defined for Formula Ia.

Formula I also includes compounds of Formula IIa:

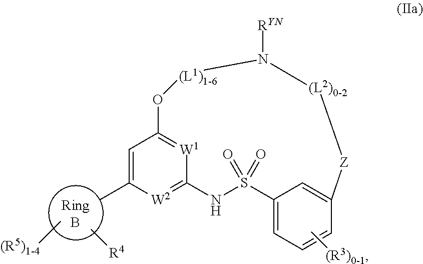

tautomers of those compounds, deuterated derivatives of any of the compounds and tautomers, and pharmaceutically acceptable salts of any of the foregoing, wherein all variables are as defined for Formula Ia.

Formula I also includes compounds of Formula IIb:

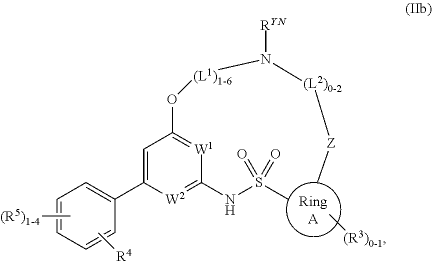

tautomers of those compounds, deuterated derivatives of any of the compounds and tautomers, and pharmaceutically acceptable salts of any of the foregoing, wherein all variables are as defined for Formula Ia.

Formula I also includes compounds of Formula III:

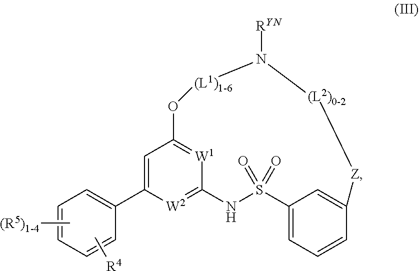

tautomers of those compounds, deuterated derivatives of any of the compounds and tautomers, and pharmaceutically acceptable salts of any of the foregoing, wherein all variables are as defined for Formula Ia.

Formula I also includes compounds of Formula IV:

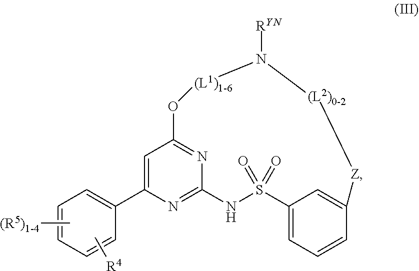

tautomers of those compounds, deuterated derivatives of any of the compounds and tautomers, and pharmaceutically acceptable salts of any of the foregoing, wherein all variables are as defined for Formula Ia.

Formula I also includes compounds of Formula V:

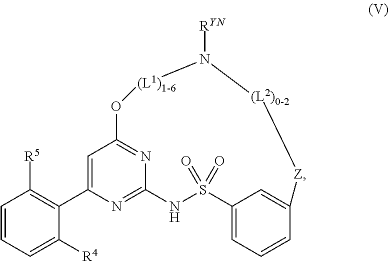

tautomers of those compounds, deuterated derivatives of any of the compounds and tautomers, and pharmaceutically acceptable salts of any of the foregoing, wherein all variables are as defined for Formula Ia.

Formula I also includes compounds of Formula VI.

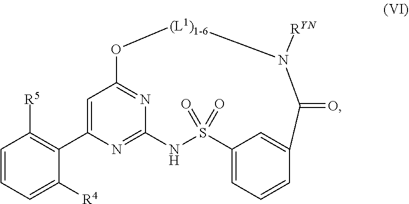

tautomers of those compounds, deuterated derivatives of any of the compounds and tautomers, and pharmaceutically acceptable salts of any of the foregoing, wherein all variables are as defined for Formula Ia.

Another aspect of the disclosure provides pharmaceutical compositions comprising at least one compound chosen from the novel compounds disclosed herein, tautomers thereof, deuterated derivatives of those compounds and tautomers, and pharmaceutically acceptable salts of any of the foregoing, and at least one pharmaceutically acceptable carrier, which compositions may further include at least one additional active pharmaceutical ingredient. In some embodiments of the pharmaceutical compositions disclosed herein, the at least one additional active pharmaceutical ingredient is at least one other CFTR modulator. In some embodiments, the at least one other CFTR modulator is selected from CFTR potentiators and CFTR modulators.

Thus, another aspect of the disclosure provides methods of treating the CFTR-mediated disease cystic fibrosis comprising administering at least one compound chosen from the novel compounds disclosed herein, tautomers thereof, deuterated derivatives of those compounds and tautomers, and pharmaceutically acceptable salts of any of the foregoing, and at least one pharmaceutically acceptable carrier, optionally as part of a pharmaceutical composition comprising at least one additional component, to a subject in need thereof. In some embodiments, the at least one additional active pharmaceutical ingredient in the methods of treating disclosed herein is at least one other CFTR modulator. In some embodiments, the at least one other CFTR modulator is selected from CFTR potentiators and CFTR correctors.

In certain embodiments, the pharmaceutical compositions of the disclosure comprise at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing. In some embodiments, compositions comprising at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing may optionally further comprise (a) at least one (i.e., one or more) compound chosen from (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (tezacaftor), 3-(6-(1-(2,2-difluorobenzo[d][1,3]dioxol-5-yl)cyclopropane carboxamido)-3-methylpyridin-2-yl)benzoic acid (lumacaftor), deuterated derivatives of tezacaftor and lumacaftor, and pharmaceutically acceptable salts of any of the foregoing; and/or (b) at least one (i.e., one or more) compound chosen from N-[2,4-bis(1,1-dimethylethyl)-5-hydroxyphenyl]-1,4-dihydro-4-oxoquinoline-3-carboxamide (ivacaftor), N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (deutivacaftor), (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, deuterated derivatives of ivacaftor, deutivacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and pharmaceutically acceptable salts of any of the foregoing.

Another aspect of the disclosure provides methods of treating the CFTR-mediated disease cystic fibrosis comprising administering to a patient in need thereof at least one compound chosen from the novel compounds disclosed herein, pharmaceutically acceptable salts thereof, and deuterated derivatives of any of the foregoing, and optionally further administering one or more additional CFTR modulating agents selected from tezacaftor, ivacaftor, and lumacaftor.

In a further aspect, compounds of the disclosure (e.g., compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing) and pharmaceutical compositions comprising those compounds, and optionally further comprising one or more CFTR modulating agents, are used in therapy or in the manufacture of a medicament. In some embodiments, the one or more additional CFTR modulating agents are selected from CFTR potentiators. In some embodiments, the one or more additional CFTR modulating agents are selected from CFTR correctors. In some embodiments, the one or more additional CFTR modulating agents are selected from tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

A further aspect of the disclosure provides intermediates and methods for making the compounds and compositions disclosed herein.

### Definitions

“Tezacaftor” as used herein, refers to (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide, which can be depicted with the following structure:

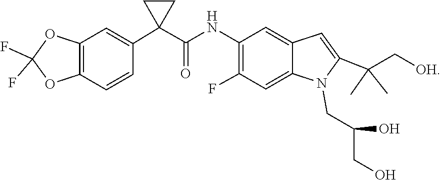 [SMILES: CC(C)(CO)c1cc2cc(NC(=O)C3(c4ccc5c(c4)OC(F)(F)O5)CC3)c(F)cc2n1C[C@@H](O)CO]

Tezacaftor may be in the form of a deuterated derivative, a pharmaceutically acceptable salt, or a pharmaceutically acceptable salt of a deuterated derivative. Tezacaftor and methods of making and using tezacaftor are disclosed in WO 2010/053471, WO 2011/119984, WO 2011/133751, WO 2011/133951, WO 2015/160787, and US 2009/0131492, each of which is incorporated herein by reference.

“Ivacaftor” as used throughout this disclosure refers to N-[2,4-bis(1,1-dimethylethyl)-5-hydroxyphenyl]-1,4-dihydro-4-oxoquinoline-3-carboxamide, which is depicted by the structure:

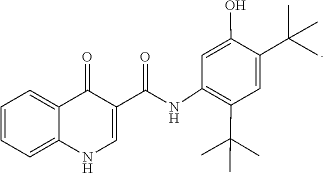 [SMILES: CC(C)(C)c1cc(C(C)(C)C)c(NC(=O)c2c[nH]c3ccccc3c2=O)cc1O]

Ivacaftor may also be in the form of a deuterated derivative, a pharmaceutically acceptable salt, or a pharmaceutically acceptable salt of a deuterated derivative. Ivacaftor and methods of making and using ivacaftor are disclosed in WO 2006/002421, WO 2007/079139, WO 2010/108162, and WO 2010/019239, each of which is incorporated herein by reference.

In some embodiments, a deuterated derivative of ivacaftor (deutivacaftor) is employed in the compositions and methods disclosed herein. A chemical name for deutivacaftor is N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide, as depicted by the structure:

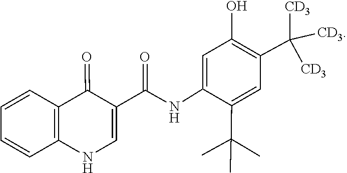 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

Deutivacaftor may be in the form of a further deuterated derivative, a pharmaceutically acceptable salt, or a pharmaceutically acceptable salt of a deuterated derivative. Deutivacaftor and methods of making and using deutivacaftor are disclosed in WO 2012/158885, WO 2014/078842, and U.S. Pat. No. 8,865,902, each of which is incorporated herein by reference.

“Lumacaftor” as used herein, refers to 3-(6-(1-(2,2-difluorobenzo[d][1,3]dioxol-5-yl)cyclopropanecarboxamido)-3-methylpyridin-2-yl)benzoic acid, which is depicted by the chemical structure:

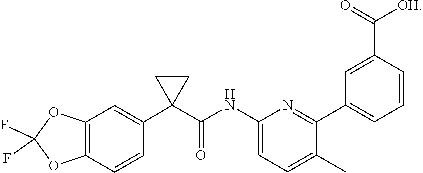 [SMILES: Cc1ccc(NC(=O)C2(c3ccc4c(c3)OC(F)(F)O4)CC2)nc1-c1cccc(C(=O)O)c1]

Lumacaftor may be in the form of a deuterated derivative, a pharmaceutically acceptable salt, or a pharmaceutically acceptable salt of a deuterated derivative. Lumacaftor and methods of making and using lumacaftor are disclosed in WO 2007/056341, WO 2009/073757, and WO 2009/076142, each of which is incorporated herein by reference.

As used herein, the term “alkyl” refers to a saturated or partially saturated, branched, or unbranched aliphatic hydrocarbon containing carbon atoms (such as, for example, 1, 2, 3, 4, 5, 6, 7, 8, 9, 10, 11, 12, 13, 14, 15, 16, 17, 18, 19, or 20 carbon atoms) in which one or more adjacent carbon atoms is interrupted by a double (alkenyl) or triple (alkynyl) bond. Alkyl groups may be substituted or unsubstituted.

As used herein, the term “haloalkyl group” refers to an alkyl group substituted with one or more halogen atoms, e.g., fluoroalkyl, which refers to an alkyl group substituted with one or more fluorine atoms.

The term “alkoxy,” as used herein, refers to an alkyl or cycloalkyl covalently bonded to an oxygen atom. Alkoxy groups may be substituted or unsubstituted.

As used herein, the term “haloalkoxyl group” refers to an alkoxy group substituted with one or more halogen atoms.

As used herein, “cycloalkyl” refers to a cyclic, bicyclic, tricyclic, or polycyclic non-aromatic hydrocarbon groups having 3 to 12 carbons (such as, for example 3-10 carbons) and may include one or more unsaturated bonds. “Cycloalkyl” groups encompass monocyclic, bicyclic, tricyclic, bridged, fused, and spiro rings, including mono spiro and dispiro rings. Non-limiting examples of cycloalkyl groups are cyclopropyl, cyclobutyl, cyclopentyl, cyclohexyl, adamantyl, norbornyl, dispiro[2.0.2.1]heptane, and spiro[2,3]hexane. Cycloalkyl groups may be substituted or unsubstituted.

The term “aryl,” as used herein, is a functional group or substituent derived from an aromatic ring and encompasses monocyclic aromatic rings and bicyclic, tricyclic, and fused ring systems wherein at least one ring in the system is aromatic. Non-limiting examples of aryl groups include phenyl, naphthyl, and 1,2,3,4-tetrahydronaphthalenyl.

The term “heteroaryl ring,” as used herein, refers to an aromatic ring comprising at least one ring atom that is a heteroatom, such as O, N, or S. Heteroaryl groups encompass monocyclic rings and bicyclic, tricyclic, bridged, fused, and spiro ring systems (including mono spiro and dispiro rings) wherein at least one ring in the system is aromatic. Non-limiting examples of heteroaryl rings include pyridine, quinoline, indole, and indoline. In certain embodiments, the term “heteroaryl ring” encompasses heteroaryl rings with various oxidation states, such as heteroaryl rings containing N-oxides and sulfoxides. Non-limiting examples of such heteroaryl rings include pyrimidine N-oxides, quinoline N-oxides, thiophene S-oxides, and pyrimidine N-oxides.

As used herein, the term “heterocyclyl ring” refers to a non-aromatic hydrocarbon containing 3 to 12 atoms in a ring (such as, for example 3-10 atoms) comprising at least one ring atom that is a heteroatom, such as O, N, or S, and may include one or more unsaturated bonds. “Heterocyclyl” rings encompass monocyclic, bicyclic, tricyclic, polycyclic, bridged, fused, and spiro rings, including mono spiro and dispiro rings.

“Substituted,” whether preceded by the term “optionally” or not, indicates that at least one hydrogen of the “substituted” group is replaced by a substituent. Unless otherwise indicated, an “optionally substituted” group may have a suitable substituent at each substitutable position of the group, and when more than one position in any given structure may be substituted with more than one substituent chosen from a specified group, the substituent may be either the same or different at each position.

Non-limiting examples of protecting groups for nitrogen include, for example, t-butyl carbamate (Boc), benzyl (Bn), para-methoxybenzyl (PMB), tetrahydropyranyl (THP), 9-fluorenylmethyl carbamate (Fmoc), benzyl carbamate (Cbz), methyl carbamate, ethyl carbamate, 2,2,2-trichloroethyl carbamate (Troc), 2-trimethylsilylethyl carbamate (Teoc), allyl carbamate (Aloc or Alloc), formamide, acetamide, benzamide, allylamine, trifluoroacetamide, triphenylmethylamine, benzylideneamine, and p-toluenesulfonamide. A comprehensive list of nitrogen protecting groups can be found in Wuts, P. G. M. “Greene's Protective Groups in Organic Synthesis: Fifth Edition,” 2014, John Wiley and Sons.

As used herein, “deuterated derivative(s)” refers to a compound having the same chemical structure as a reference compound, with one or more hydrogen atoms replaced by a deuterium atom. In some embodiments, the one or more hydrogens replaced by deuterium are part of an alkyl group. In some embodiments, the one or more hydrogens replaced by deuterium are part of a methyl group.

The phrase “and deuterated derivatives and pharmaceutically acceptable salts thereof” is used interchangeably with “and deuterated derivatives and pharmaceutically acceptable salts thereof of any of the forgoing” in reference to one or more specified compounds. These terms, as used herein, are intended to include deuterated derivatives of the specified compound or compounds and pharmaceutically acceptable salts of the specified compound or compounds, as well as pharmaceutically acceptable salts of deuterated derivatives of the specified compound or compounds.

As used herein, “CFTR” means cystic fibrosis transmembrane conductance regulator.

As used herein, the term “CFTR modulator” refers to a compound that increases the activity of CFTR. The increase in activity resulting from a CFTR modulator includes, but is not limited to, compounds that correct, potentiate, stabilize, and/or amplify CFTR.

As used herein, the terms “corrector” and “CFTR corrector” are used interchangeably and refer to a compound that facilitates the processing and trafficking of CFTR to increase the amount of CFTR at the cell surface. The novel compounds disclosed herein are CFTR correctors. Tezacaftor and lumacaftor, and deuterated derivatives and pharmaceutically acceptable salts thereof as referenced herein, are correctors.

As used herein, the terms “potentiator” and “CFTR potentiator” refer to a compound that increases the channel activity of CFTR protein located at the cell surface, resulting in enhanced ion transport. Ivacaftor, deutivacaftor, and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, as referenced herein, are CFTR potentiators. It will be appreciated that when a description of a combination of compounds selected from compounds of Formula I, compounds of any of Formulae I, Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, the combination will typically but not necessarily include a CFTR potentiator, such as, e.g., ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, or a deuterated derivative or pharmaceutically acceptable salt of any of the foregoing. In addition, the combination will typically, but not necessarily, include only a single potentiator, but may include more than one corrector. Thus, in some embodiments, a combination of at least one compound selected from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, will include a potentiator selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, or deuterated derivatives or pharmaceutically acceptable salts thereof and may also include another CFTR corrector, such as, e.g., a corrector compound selected from tezacaftor, lumacaftor, and deuterated derivatives and pharmaceutically acceptable salts thereof.

The term “at least one compound selected from,” as used herein, refers to the selection of one or more of the compounds from a specified group.

A reference to “Compounds 1-1924” herein is intended to represent a reference to each of Compounds 1 through 1294 individually or a reference to groups of compounds, such as, e.g., Compounds 1-1193, Compounds 1194-1294, and Compounds 1295-1972.

As used herein, the term “active pharmaceutical ingredient” or “therapeutic agent” (“API”) refers to a biologically active compound.

The terms “patient” and “subject” are used interchangeably and refer to an animal, including a human.

The terms “effective dose” and “effective amount” are used interchangeably herein and refer to that amount of a compound that produces the desired effect for which it is administered (e.g., improvement in CF or a symptom of CF, or lessening the severity of CF or a symptom of CF). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement in one or more symptoms of CF or lessening the severity of CF or one or more symptoms of CF in a subject. “Treatment,” as used herein, includes, but is not limited to, the following: increased growth of the subject, increased weight gain, reduction of mucus in the lungs, improved pancreatic and/or liver function, reduction of chest infections, and/or reductions in coughing or shortness of breath. Improvements in or lessening the severity of any of these symptoms can be readily assessed according to standard methods and techniques known in the art.

It should be understood that references herein to methods of treatment (e.g., methods of treating a CFTR mediated disease or a method of treating cystic fibrosis) using one or more compounds of the disclosure optionally in combination with one or more additional CFTR modulating agents (e.g., a compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, optionally in combination with one or more additional CFTR modulating agents) should also be interpreted as references to:
one or more compounds (e.g., a compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, optionally in combination with one or more additional CFTR modulating agents) for use in methods of treating, e.g., cystic fibrosis, optionally in combination with one or more additional CFTR modulating agents; and/or
the use of one or more compounds (e.g., a compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, optionally in combination with one or more additional CFTR modulating agents) in the manufacture of a medicament for treating, e.g., cystic fibrosis.

It should be also understood that references herein to methods of treatment (e.g., methods of treating a CFTR mediated disease or a method of treating cystic fibrosis) using a pharmaceutical composition of the disclosure (e.g., a pharmaceutical composition comprising at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and optionally further comprising one or more additional CFTR modulating agents) should also be interpreted as references to:
a pharmaceutical composition (e.g., a pharmaceutical composition comprising at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and optionally further comprising one or more additional CFTR modulating agents) for use in methods of treating, e.g., cystic fibrosis; and/or
the use of a pharmaceutical composition (e.g., a pharmaceutical composition comprising at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and optionally further comprising one or more additional CFTR modulating agents) in the manufacture of a medicament for treating, e.g., cystic fibrosis.

As used herein, the term “in combination with,” when referring to two or more compounds, agents, or additional active pharmaceutical ingredients, means the administration of two or more compounds, agents, or active pharmaceutical ingredients to the patient prior to, concurrent with, or subsequent to each other.

The terms “about” and “approximately” may refer to an acceptable error for a particular value as determined by one of skill in the art, which depends in part on how the values are measured or determined. In some embodiments, the terms “about” and “approximately” mean within 20%, 15%, 10%, 5%, 4%, 3%, 2%, 1%, or 0.5% of a given value or range.

As used herein, the term “solvent” refers to any liquid in which the product is at least partially soluble (solubility of product>1 g/L).

As used herein, the term “room temperature” or “ambient temperature” means 15° C. to 30° C.

It will be appreciated that certain compounds of this disclosure may exist as separate stereoisomers or enantiomers and/or mixtures of those stereoisomers or enantiomers.

Certain compounds disclosed herein may exist as tautomers and both tautomeric forms are intended, even though only a single tautomeric structure is depicted. For example, a description of Compound X is understood to include its tautomer Compound Y and vice versa, as well as mixtures thereof:

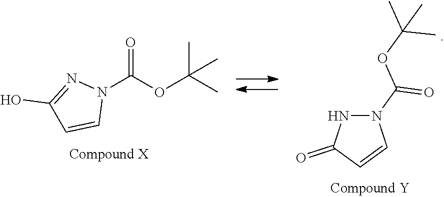

As used herein, “minimal function (MF) mutations” refer to CFTR gene mutations associated with minimal CFTR function (little-to-no functioning CFTR protein) and include, for example, mutations associated with severe defects in ability of the CFTR channel to open and close, known as defective channel gating or “gating mutations”; mutations associated with severe defects in the cellular processing of CFTR and its delivery to the cell surface; mutations associated with no (or minimal) CFTR synthesis; and mutations associated with severe defects in channel conductance.

As used herein, the term “pharmaceutically acceptable salt” refers to a salt form of a compound of this disclosure, wherein the salt is nontoxic. Pharmaceutically acceptable salts of the compounds of this disclosure include those derived from suitable inorganic and organic acids and bases. A “free base” form of a compound, for example, does not contain an ionically bonded salt.

One of ordinary skill in the art would recognize that, when an amount of “a compound or a pharmaceutically acceptable salt thereof” is disclosed, the amount of the pharmaceutically acceptable salt form of the compound is the amount equivalent to the concentration of the free base of the compound. It is noted that the disclosed amounts of the compounds or their pharmaceutically acceptable salts thereof herein are based upon their free base form.

Suitable pharmaceutically acceptable salts are, for example, those disclosed in S. M. Berge, et al. J. Pharmaceutical Sciences, 1977, 66, 1-19. For example, Table 1 of that article provides the following pharmaceutically acceptable salts:

TABLE 1

| | | |
|---|---|---|
| Acetate | Iodide | Benzathine |
| Benzenesulfonate | Isethionate | Chloroprocaine |
| Benzoate | Lactate | Choline |
| Bicarbonate | Lactobionate | Diethanolamine |
| Bitartrate | Malate | Ethylenediamine |
| Bromide | Maleate | Meglumine |
| Calcium edetate | Mandelate | Procaine |
| Camsylate | Mesylate | Aluminum |
| Carbonate | Methylbromide | Calcium |
| Chloride | Methylnitrate | Lithium |
| Citrate | Methylsulfate | Magnesium |
| Dihydrochloride | Mucate | Potassium |
| Edetate | Napsylate | Sodium |
| Edisylate | Nitrate | Zinc |
| Estolate | Pamoate (Embonate) | |
| Esylate | Pantothenate | |
| Fumarate | Phosphate/diphosphate | |
| Gluceptate | Polygalacturonate | |
| Gluconate | Salicylate | |
| Glutamate | Stearate | |
| Glycollylarsanilate | Subacetate | |
| Hexylresorcinate | Succinate | |
| Hydrabamine | Sulfate | |
| Hydrobromide | Tannate | |
| Hydrochloride | Tartrate | |
| Hydroxynaphthoate | Teociate | |
| | Triethiodide | |

Non-limiting examples of pharmaceutically acceptable acid addition salts include: salts formed with inorganic acids, such as hydrochloric acid, hydrobromic acid, phosphoric acid, sulfuric acid, or perchloric acid; salts formed with organic acids, such as acetic acid, oxalic acid, maleic acid, tartaric acid, citric acid, succinic acid, or malonic acid; and salts formed by using other methods used in the art, such as ion exchange. Non-limiting examples of pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts. Pharmaceutically acceptable salts derived from appropriate bases include alkali metal, alkaline earth metal, ammonium, and N⁺ (C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

“Selected from” and “chosen from” are used interchangeably herein.

### Methods of Treatment

Any of the novel compounds disclosed herein, such as, for example, compounds of Formula I, compounds of any of Formulae Ia, Ib, IIa, IIb, III, IV, V, and VI, Compounds 1-1193, Compounds 1194-1294, Compounds 1295-1972, deuterated derivatives thereof, and pharmaceutically acceptable salts of any of the foregoing, can act as a CFTR modulator, i.e., it modulates CFTR activity in the body. Individuals suffering from a mutation in the gene encoding CFTR may benefit from receiving a CFTR modulator. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect). Some CFTR mutations exhibit characteristics of multiple classes. Certain mutations in the CFTR gene result in cystic fibrosis.

Thus, in some embodiments, the disclosure provides methods of treating, lessening the severity of, or symptomatically treating cystic fibrosis in a patient comprising administering to the patient an effective amount of any of the novel compounds disclosed herein, such as, for example, compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, alone or in combination with another active ingredient, such as one or more CFTR modulating agents. In some embodiments, the one or more CFTR modulating agents are selected from ivacaftor, deutivacaftor, lumacaftor, and tezacaftor. In some embodiments, the patient has an F508del/minimal function (MF) genotype, F508del/F508del genotype (homozygous for the F508del mutation), F508del/gating genotype, or F508del/residual function (R^{F}) genotype. In some embodiments, the patient is heterozygous and has one F508del mutation. In some embodiments, the patient is homozygous for the N1303K mutation.

In some embodiments, 5 mg to 500 mg of a compound disclosed herein, a tautomer thereof, deuterated derivatives of the compound and tautomer, or a pharmaceutically acceptable salt of any of the foregoing are administered daily.

In some embodiments, the patient has at least one F508del mutation in the CFTR gene. In some embodiments, the patient has a CFTR gene mutation that is responsive to a compound, tautomer, deuterated derivative, or pharmaceutically acceptable salt of the invention based on in vitro data. In some embodiments, the patient is heterozygous and has an F508del mutation on one allele and a mutation on the other allele selected from Table 2:

TABLE 2
CFTR Mutations

| MF Category | Mutation | | | | |
|---|---|---|---|---|---|
| Nonsense mutations | Q2X | L218X | Q525X | R792X | E1104X |
| | S4X | Q220X | G542X | E822X | W1145X |
| | W19X | Y275X | G550X | W882X | R1158X |
| | G27X | C276X | Q552X | W846X | R1162X |
| | Q39X | Q290X | R553X | Y849X | S1196X |
| | W57X | G330X | E585X | R851X | W1204X |
| | E60X | W401X | G673X | Q890X | L1254X |
| | R75X | Q414X | Q685X | S912X | S1255X |
| | L88X | S434X | R709X | Y913X | W1282X |
| | E92X | S466X | K710X | Q1042X | Q1313X |
| | Q98X | S489X | Q715X | W1089X | Q1330X |
| | Y122X | Q493X | L732X | Y1092X | E1371X |
| | E193X | W496X | R764X | W1098X | Q1382X |
| | W216X | C524X | R785X | R1102X | Q1411X |
| Canonical splice mutations | 185 + 1G→T | 711 + 5G→A | 1717-8G→A | 2622 + 1G→A | 3121-1G→A |
| | 296 + 1G→A | 712-1G→T | 1717-1G→A | 2790-1G→C | 3500-2A→G |
| | 296 + 1G→T | 1248 + 1G→A | 1811 + 1G→C | 3040G→C | 3600 + 2insT |
| | 405 + 1G→A | 1249-1G→A | 1811 + 1.6kbA→G | (G970R) | 3850-1G→A |
| | 405 + 3A→C | 1341 + 1G→A | 1811 + 1643G→T | 3120G→A | 4005 + 1G→A |
| | 406 - 1G→A | 1525 - 2A→G | 1812-1G→A | 3120 + 1G→A | 4374 + 1G→T |
| | 621 + 1G→T | 1525 - 1G→A | 1898 + 1G→A | 3121-2A→G | |
| | 711 + 1G→T | | 1898 + 1G→C | | |
| Small (≤3 nucleotide) | 182delT | 1078delT | 1677delTA | 2711delT | 3737delA |
| insertion/deletion (ins/del) | 306insA | 1119delA | 1782delA | 2732insA | 3791delC |
| frameshift mutations | 306delTAGA | 1138insG | 1824delA | 2869insG | 3821delT |
| | 365-366insT | 1154insTC | 1833delT | 2896insAG | 3876delA |
| | 394delTT | 1161delC | 2043delG | 2942insT | 3878delG |
| | 442delA | 1213delT | 2143delT | 2957delT | 3905insT |
| | 444delA | 1259insA | 2183AA→Gª | 3007delG | 4016insT |
| | 457TAT→G | 1288insTA | 2184delA | 3028delA | 4021dupT |
| | 541delC | 1343delG | 2184insA | 3171delC | 4022insT |
| | 574delA | 1471delA | 2307insA | 3171insC | 4040delA |
| | 663delT | 1497delGG | 2347delG | 3271delGG | 4279insA |
| | 849delG | 1548delG | 2585delT | 3349insT | 4326delTC |
| | 935delA | 1609del CA | 2594delGT | 3659delC | |

| Non-small (>3 nucleotide) | CFTRdele2 | CFTRdele 16-17b | 1461ins4 | |
|---|---|---|---|---|
| insertion/deletion (ins/del) | CFTRdele2 | CFTRdele 17a,17b | 1924del7 | |
| frameshift mutations | CFTRdele2,3 | CFTRdele 17a-18 | 2055del9→A | |
| | CFTRdele2-4 | CFTRdele19 | 2105-2117de113insAGAAA | |
| | CFTRdele3-10,14b-16 | CFTRdele21 | 2372de19 | |
| | CFTRdele4-7 | CFTRdele21 | 2721del11 | |
| | CFTRdele4-11 | CFTRdel22,23 | 2991del32 | |
| | CFTR50kbdel | CFTRdele22,23 | 3667ins4 | |
| | CFTRdup6b-10 | 124del23bp | 4010del4 | |
| | CFTRdele11 | 602del14 | 4209TGTT→AA | |
| | CFTRdele13,14a | 85sdel22 | | |
| | CFTRdele14b-17b | 991del5 | | |
| Missense mutations that | A46D | V520F | Y569D | N1303K |
| · Are not responsive in | G85E | A559T | L1065P | |
| vitro to TEZ, IVA, or | R347P | R560T | R1066C | |
| TEZ/IVA | L467P | R560S | L1077P | |
| and % PI >50% and | I507del | A561E | M1101K | |
| SwCl⁻ >86 mmol/L | | | | |

^{a}Also known as 2183delAA→G.
CFTR: cystic fibrosis transmembrane conductance regulator;
IVA: ivacaftor.
SwCl: sweat chloride.
TEZ: tezacaftor.
Source: CFTR2.org [Internet] . Baltimore (MD): Clinical and functional translation of CFTR. The Clinical and
Functional Translation of CFTR (CFTR2), US Cystic Fibrosis Foundation, Johns Hopkins University, the
Hospital for Sick Children. Available at: http://www.cftr2.org/. Accessed 15 May 2018.
Notes: % PI: percentage of F508del-CFTR heterozygous patients in the CFTR2 patient registry who are pancreatic
insufficient; SwCl: mean sweat chloride of F508del-CFTR heterozygous patients in the CFTR2 patient registry.

In some embodiments, the disclosure also is directed to methods of treatment using isotope-labelled compounds of the afore-mentioned compounds, or pharmaceutically acceptable salts thereof, wherein the formula and variables of such compounds and salts are each and independently as described above or any other embodiments described above, provided that one or more atoms therein have been replaced by an atom or atoms having an atomic mass or mass number which differs from the atomic mass or mass number of the atom which usually occurs naturally (isotope labelled). Examples of isotopes which are commercially available and suitable for the disclosure include isotopes of hydrogen, carbon, nitrogen, oxygen, phosphorus, fluorine, and chlorine, for example ²H, ³H, ¹³C, ¹⁴C, ¹⁵N, ¹⁸O, ¹⁷O, ³¹P, ³²p, ³⁵S, ¹⁸F, and ³⁶Cl, respectively.

The isotope-labelled compounds and salts can be used in a number of beneficial ways. They can be suitable for medicaments and/or various types of assays, such as substrate tissue distribution assays. For example, tritium (3H)- and/or carbon-14 (¹⁴C)-labelled compounds are particularly useful for various types of assays, such as substrate tissue distribution assays, due to relatively simple preparation and excellent detectability. For example, deuterium (²H)-labelled ones are therapeutically useful with potential therapeutic advantages over the non-²H-labelled compounds. In general, deuterium (²H)-labelled compounds and salts can have higher metabolic stability as compared to those that are not isotope-labelled owing to the kinetic isotope effect described below. Higher metabolic stability translates directly into an increased in vivo half-life or lower dosages, which could be desired. The isotope-labelled compounds and salts can usually be prepared by carrying out the procedures disclosed in the synthesis schemes and the related description, in the example part and in the preparation part in the present text, replacing a non-isotope-labelled reactant by a readily available isotope-labelled reactant.

In some embodiments, the isotope-labelled compounds and salts are deuterium (2H)-labelled ones. In some specific embodiments, the isotope-labelled compounds and salts are deuterium (²H)-labelled, wherein one or more hydrogen atoms therein have been replaced by deuterium. In chemical structures, deuterium is represented as “D.”

The concentration of the isotope(s) (e.g., deuterium) incorporated into the isotope-labelled compounds and salt of the disclosure may be defined by the isotopic enrichment factor. The term “isotopic enrichment factor” as used herein means the ratio between the isotopic abundance and the natural abundance of a specified isotope. In some embodiments, if a substituent in a compound of the disclosure is denoted as deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Combination Therapies

One aspect disclosed herein provides methods of treating cystic fibrosis and other CFTR mediated diseases using any of the novel compounds disclosed herein, such as, for example, compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, in combination with at least one additional active pharmaceutical ingredient.

In some embodiments, at least one additional active pharmaceutical ingredient is selected from mucolytic agents, bronchodilators, antibiotics, anti-infective agents, and anti-inflammatory agents.

In some embodiments, the additional therapeutic agent is an antibiotic. Exemplary antibiotics useful herein include tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.

In some embodiments, the additional agent is a mucolyte. Exemplary mucolytes useful herein include Pulmozyme®.

In some embodiments, the additional agent is a bronchodilator. Exemplary bronchodilators include albuterol, metaprotenerol sulfate, pirbuterol acetate, salmeterol, or tetrabuline sulfate.

In some embodiments, the additional agent is an anti-inflammatory agent, i.e., an agent that can reduce the inflammation in the lungs. Exemplary such agents useful herein include ibuprofen, docosahexanoic acid (DHA), sildenafil, inhaled glutathione, pioglitazone, hydroxychloroquine, or simavastatin.

In some embodiments, the additional agent is a nutritional agent. Exemplary nutritional agents include pancrelipase (pancreatic enzyme replacement), including Pancrease®, Pancreacarb®, Ultrase®, or Creon®, Liprotomase® (formerly Trizytek®), Aquadeks®, or glutathione inhalation. In some embodiments, the additional nutritional agent is pancrelipase.

In some embodiments, at least one additional active pharmaceutical ingredient is selected from CFTR modulating agents. In some embodiments, the at least one additional active pharmaceutical ingredient is selected from CFTR potentiators. In some embodiments, the potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In some embodiments, the at least one additional active pharmaceutical ingredient is chosen from CFTR correctors. In some embodiments, the correctors are selected from lumacaftor, tezacaftor, and deuterated derivatives and pharmaceutically acceptable salts of any of the foregoing.

In some embodiments, the at least one additional active pharmaceutical ingredient is chosen from (a) tezacaftor, lumacaftor, and deuterated derivatives and pharmaceutically acceptable salts thereof; and/or (b) ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.

Thus, in some embodiments, the combination therapies provided herein comprise (a) a compound selected from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing; and (b) at least one compound selected from tezacaftor, lumacaftor, and deuterated derivatives and pharmaceutically acceptable salts thereof; or (c) at least one compound selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing. In other embodiments, the combination therapies provided herein comprise (a) at least one compound chosen from compounds of Formula I, compounds of any of Formulae Ia, Ib, IIa, IIb, III, IV, V, and VI, Compounds 1-1193, Compounds 1194-1294, Compounds 1295-1972, deuterated derivatives thereof, and pharmaceutically acceptable salts of any of the foregoing; (b) at least one compound selected from tezacaftor and pharmaceutically acceptable salts thereof; and (c) at least one compound selected from ivacaftor, deutivacaftor, and pharmaceutically acceptable salts thereof. In still other embodiments, the combination therapies provided herein comprise (a) at least one compound chosen from compounds of Formula I, compounds of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing; (b) at least one compound selected from tezacaftor, lumacaftor, and deuterated derivatives and pharmaceutically acceptable salts thereof, and/or (c) at least one compound selected from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in combination with at least one compound chosen from tezacaftor and pharmaceutically acceptable salts thereof. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in combination with at least one compound chosen from lumacaftor and pharmaceutically acceptable salts thereof. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in combination with at least one compound chosen from ivacaftor and pharmaceutically acceptable salts thereof. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, deuterated derivatives thereof, and pharmaceutically acceptable salts of any of the foregoing, is administered in combination with at least one compound chosen from deutivacaftor and pharmaceutically acceptable salts thereof. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in combination with at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and pharmaceutically acceptable salts thereof.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing is administered in combination with at least one compound chosen from tezacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in combination with at least one compound chosen from tezacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing is administered in combination with at least one compound chosen from lumacaftor and deuterated derivatives and pharmaceutically acceptable salts thereof and at least one compound chosen from ivacaftor and deuterated derivatives and pharmaceutically acceptable salts thereof. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from deutivacaftor and deuterated derivatives and pharmaceutically acceptable salts thereof. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in combination with at least one compound chosen from lumacaftor and deuterated derivatives and pharmaceutically acceptable salts thereof and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof.

Each of the compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, independently can be administered once daily, twice daily, or three times daily. In some embodiments, at least one compound chosen from compounds of Formula I, compounds of any of Formulae Ia, IIa, IIb, III, IV, V, and VI, Compounds 1-1193, Compounds 1194-1294, tautomers thereof, deuterated derivatives of those compounds and tautomers, and pharmaceutically acceptable salts of any of the foregoing, is administered once daily. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered twice daily.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered once daily. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof are administered twice daily.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing are administered once daily. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof are administered twice daily.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, at least one compound chosen from tezacaftor and deuterated derivatives and pharmaceutically acceptable salts thereof, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof are administered once daily. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, at least one compound chosen from tezacaftor and pharmaceutically acceptable salts thereof, and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof are administered twice daily.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof, and at least one compound chosen from lumacaftor and deuterated derivatives and pharmaceutically acceptable salts thereof, are administered once daily. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof, and at least one compound chosen from lumacaftor and deuterated derivatives and pharmaceutically acceptable salts thereof, are administered twice daily.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof, are administered once daily and at least one compound chosen from ivacaftor and deuterated derivatives and pharmaceutically acceptable salts thereof, are administered twice daily. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and at least one compound chosen from lumacaftor and pharmaceutically acceptable salts thereof, are administered once daily and at least one compound chosen from ivacaftor and pharmaceutically acceptable salts thereof, are administered twice daily.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and at least one compound chosen from tezacaftor and deuterated derivatives and pharmaceutically acceptable salts thereof, are administered once daily and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, are administered once or twice daily. In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and at least one compound chosen from lumacaftor and deuterated derivatives and pharmaceutically acceptable salts thereof, are administered once daily and at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, are administered once or twice daily.

Compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, tezacaftor, lumacaftor, ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and their deuterated derivatives and pharmaceutically acceptable salts thereof can be administered in a single pharmaceutical composition or separate pharmaceutical compositions. Such pharmaceutical compositions can be administered once daily or multiple times daily, such as twice daily. As used herein, the phrase that a given amount of API (e.g., tezacaftor, lumacaftor, ivacaftor, deutivacaftor (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof) is administered once or twice daily or per day means that said given amount is administered per dosing once or twice daily.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; and at least one compound chosen from ivacaftor and deuterated derivatives and pharmaceutically acceptable salts thereof is administered in a third pharmaceutical composition.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from deutivacaftor and deuterated derivatives and pharmaceutically acceptable salts thereof is administered in a third pharmaceutical composition.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in a first pharmaceutical composition; at least one compound chosen from tezacaftor and deuterated derivatives and pharmaceutically acceptable salts thereof is administered in a second pharmaceutical composition; at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof is administered in a third pharmaceutical composition.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in a first pharmaceutical composition; at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof is administered in a second pharmaceutical composition; at least one compound chosen from lumacaftor and deuterated derivatives and pharmaceutically acceptable salts thereof is administered in a third pharmaceutical composition.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, is administered in a first pharmaceutical composition; and at least one compound chosen from tezacaftor and deuterated derivatives and pharmaceutically acceptable salts thereof and at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts thereof are administered in a second pharmaceutical composition. In some embodiments, the second pharmaceutical composition comprises a half of a daily dose of said at least one compound chosen from ivacaftor and deuterated derivatives and pharmaceutically acceptable salts thereof, and the other half of the daily dose of said at least one compound chosen from ivacaftor and deuterated derivatives and pharmaceutically acceptable salts thereof is administered in a third pharmaceutical composition.

In some embodiments, at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, at least one compound chosen from tezacaftor and pharmaceutically acceptable salts thereof, and at least one compound chosen from ivacaftor, deutivacaftor, and pharmaceutically acceptable salts thereof are administered in a first pharmaceutical composition. In some embodiments, the first pharmaceutical composition is administered to the patient twice daily. In some embodiments, the first pharmaceutical composition is administered once daily. In some embodiments, the first pharmaceutical composition is administered once daily and, when the first composition comprises ivacaftor, a second composition comprising only ivacaftor is administered once daily.

Any suitable pharmaceutical compositions can be used for compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, tezacaftor, ivacaftor, deutivacaftor, lumacaftor and tautomers thereof, deuterated derivatives of those compounds and tautomers, and pharmaceutically acceptable salts of any of the foregoing. Some exemplary pharmaceutical compositions for tezacaftor and its pharmaceutically acceptable salts can be found in WO 2011/119984 and WO 2014/014841, all of which is incorporated herein by reference. Some exemplary pharmaceutical compositions for ivacaftor and its pharmaceutically acceptable salts can be found in WO 2007/134279, WO 2010/019239, WO 2011/019413, WO 2012/027731, and WO 2013/130669, and some exemplary pharmaceutical compositions for deutivacaftor and its pharmaceutically acceptable salts can be found in U.S. Pat. Nos. 8,865,902, 9,181,192, 9,512,079, WO 2017/053455, and WO 2018/080591, all of which are incorporated herein by reference. Some exemplary pharmaceutical compositions for lumacaftor and its pharmaceutically acceptable salts can be found in WO 2010/037066, WO 2011/127421, and WO 2014/071122, all of which are incorporated herein by reference.

### Pharmaceutical Compositions

Another aspect of the disclosure provides a pharmaceutical composition comprising at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and at least one pharmaceutically acceptable carrier.

In some embodiments, the disclosure provides pharmaceutical compositions comprising at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, in combination with at least one additional active pharmaceutical ingredient. In some embodiments, the at least one additional active pharmaceutical ingredient is a CFTR modulator. In some embodiments, the at least one additional active pharmaceutical ingredient is a CFTR corrector. In some embodiments, the at least one additional active pharmaceutical ingredient is a CFTR potentiator. In some embodiments, the pharmaceutical composition comprises at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, and at least two additional active pharmaceutical ingredients, one of which is a CFTR corrector and one of which is a CFTR potentiator.

In some embodiments, the disclosure provides a pharmaceutical composition comprising (a) at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, (b) at least one compound chosen from tezacaftor and pharmaceutically acceptable salts thereof, and (c) at least one pharmaceutically acceptable carrier.

In some embodiments, the disclosure provides a pharmaceutical composition comprising (a) at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, (b) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, and (c) at least one pharmaceutically acceptable carrier.

In some embodiments, the disclosure provides a pharmaceutical composition comprising (a) at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from ivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from deutivacaftor and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, (b) at least one compound chosen from tezacaftor and deuterated derivatives and pharmaceutically acceptable salts thereof, (c) at least one compound chosen from (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts thereof, and (d) at least one pharmaceutically acceptable carrier.

In some embodiments, the disclosure provides a pharmaceutical composition comprising (a) at least one compound chosen from compounds of Formula I, compounds of any of Formulae Ia, Ib, IIa, IIb, III, IV, V, and VI, Compounds 1-1193, Compounds 1194-1294, Compounds 1295-1972, tautomers thereof, deuterated derivatives of those compounds and tautomers, and pharmaceutically acceptable salts of any of the foregoing, (b) at least one compound chosen from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing, (c) at least one compound chosen from lumacaftor and deuterated derivatives and pharmaceutically acceptable salts thereof, and (d) at least one pharmaceutically acceptable carrier.

Any pharmaceutical composition disclosed herein may comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

The pharmaceutical compositions described herein are useful for treating cystic fibrosis and other CFTR mediated diseases.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose, and cellulose acetate), powdered tragacanth, malt, gelatin, talc, excipients (such as cocoa butter and suppository waxes), oils (such as peanut oil, cottonseed oil, safflower oil, sesame oil, olive oil, corn oil, and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

### Exemplary Embodiments

The following provides a non-limiting list of exemplary embodiments:

1. Formula I encompasses compounds falling within the following structure:

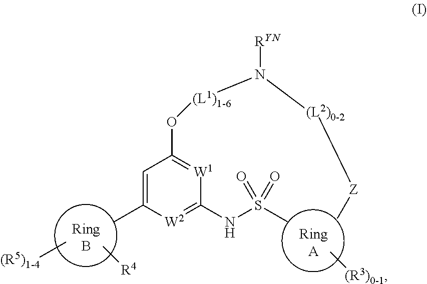

and includes tautomers thereof, deuterated derivatives of those compounds and tautomers, and pharmaceutically acceptable salts of any of the foregoing, wherein:
Ring A is selected from:
   C₆-C₁₀ aryl,
   C₃-C₁₀ cycloalkyl,
   3- to 10-membered heterocyclyl, and
   5- to 10-membered heteroaryl;
Ring B is selected from:
   C₆-C₁₀ aryl,
   C₃-C₁₀ cycloalkyl,
   3- to 10-membered heterocyclyl, and
   5- to 10-membered heteroaryl;
V is selected from O and NH;
W¹ is selected from N and CH;
W² is selected from N and CH, provided that at least one of W¹ and W² is N;
Z is selected from O, NR^{ZN}, and C(R^{ZC})₂, provided that when L² is absent, Z is C(R^{ZC})₂.
each L¹ is independently selected from C(R^{L1})₂ and

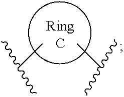

each L² is independently selected from C(R^{L2})₂;
Ring C is selected from C₆-C₁₀ aryl optionally substituted with 1-3 groups independently selected from:
   halogen,
   C₁-C₆ alkyl, and
   N(R^{N})₂;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl;
each R⁵ is independently selected from:
   hydrogen,
   halogen,
   hydroxyl,
   N(R^{N})₂,
   —SO-Me,
   —CH═C(R^{LC})₂, wherein both R^{LC} are taken together to form a C₃-C₁₀ cycloalkyl,
   C₁-C₆ alkyl optionally substituted with 1-3 groups independently selected from:
      hydroxyl,
      C₁-C₆ alkoxy optionally substituted with 1-3 groups independently selected from C₁-C₆ alkoxy and C₆-C₁₀ aryl,
      C₃-C₁₀ cycloalkyl,
      —(O)₀₋₁—(C₆-C₁₀ aryl) optionally substituted with 1-3 groups independently selected from C₁-C₆ alkyl and C₁-C₆ alkoxy,
      3- to 10-membered heterocyclyl, and
      N(R^{N})₂,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
R^{YN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy,
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl optionally substituted with 1-3 groups independently selected from oxo and C₁-C₆ alkoxy,
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl,
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3 —SiMe₃), 3- to 10-membered heterocyclyl, and N(R^{N})₂,
         C₁-C₆ alkoxy optionally substituted with 1-3 groups independently selected from hydroxyl, oxo, halogen, C₁-C₆ alkoxy, N(R^{N})₂, 3- to 10-membered heterocyclyl (optionally substituted with 1-3 groups independently selected from C₁-C₆ alkyl), and C₃-C₁₀ cycloalkyl,
         C₁-C₆ fluoroalkyl,
         —(O)₀₋₁—(C₃-C₁₀ cycloalkyl) optionally substituted with 1-3 groups independently selected from halogen, C₁-C₆ alkyl, and C₁-C₆ alkoxy,
         —(O)₀₋₁—(C₆-C₁₀ aryl),
         —(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, C₃-C₁₀ cycloalkyl, 5- to 10-membered heteroaryl (optionally substituted with 1-3 groups independently selected from C₁-C₆ alkyl), and 3- to 10-membered heterocyclyl (optionally substituted with 1-3 groups independently selected from C₁-C₆ fluoroalkyl), and
         5- to 10-membered heteroaryl optionally substituted with 1-4 groups independently selected from C₁-C₆ alkyl and C₃-C₁₀ cycloalkyl, and
   C₁-C₆ fluoroalkyl;
R^{ZN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy,
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl optionally substituted with 1-3 groups independently selected from oxo and C₁-C₆ alkoxy,
         C₁-C₆ alkoxy,
         C₁-C₆ fluoroalkyl,
         C₆-C₁₀ aryl optionally substituted with 1-3 groups independently selected from halogen, and
         5- to 10-membered heteroaryl,
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3 —SiMe₃), and N(R^{N})₂,
         C₁-C₆ alkoxy optionally substituted with 1-3 groups independently selected from hydroxyl, oxo, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl), and
         5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
         hydroxyl,
         oxo,
         N(R^{N})₂,
         C₁-C₆ alkoxy, and
         C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-member heterocyclyl optionally substituted with 1-3 groups independently selected from C₁-C₆ alkyl,
      5- to 10-membered heteroaryl optionally substituted with 1-3 groups independently selected from:
         hydroxyl,
         cyano,
         oxo,
         halogen,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl,
         3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from halogen and C₆-C₁₀ aryl, and
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
   R^{F}.
each R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl),
   C₆-C₁₀ aryl optionally substituted with 1-3 groups independently selected from C₁-C₆ alkyl, and
   R^{F};
or two R^{ZC} are taken together to form an oxo group;
each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen, C₁-C₆ alkyl, and C₁-C₆ fluoroalkyl,
      —O—(C₃-C₁₀ cycloalkyl),
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl optionally substituted with 1-3 groups independently selected from C₁-C₆ alkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
         hydroxyl,
         oxo,
         cyano,
         SiMe₃,
         N(R^{N})₂, and
         C₃-C₁₀ cycloalkyl optionally substituted with 1-3 groups independently selected from C₁-C₆ fluoroalkyl,
      C₁-C₆ alkoxy optionally substituted with 1-3 groups independently selected from:
         C₃-C₁₀ cycloalkyl optionally substituted with 1-3 groups independently selected from C₁-C₆ fluoroalkyl, and
         C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
         oxo, and
         C₁-C₆ alkoxy,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl, and
   R^{F}.
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
      NHCOMe,
      N(R^{N3})₂, wherein each R^{N3} is independently selected from C₁-C₆ alkyl (optionally substituted with 1-3 groups independently selected from oxo and C₁-C₆ alkoxy),
      C₁-C₆ alkoxy optionally substituted with 1-3 groups independently selected from C₆-C₁₀ aryl, oxo, NMe₂, and NHMe,
      —(O)₀₋₁—(C₃-C₁₀ cycloalkyl) optionally substituted with 1-3 groups independently selected from C₁-C₆ alkyl (optionally substituted with 1-3 groups independently selected from C₁-C₆ alkoxy), and C₁-C₆ alkoxy,
      C₆-C₁₀ aryl optionally substituted with 1-3 groups independently selected from halogen and C₁-C₆ alkyl,
      3- to 14-membered heterocyclyl optionally substituted with 1-4 groups independently selected from oxo and C₁-C₆ alkyl, and
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      halogen,
      NH₂,
      NHMe,
      C₁-C₆ alkoxy, and
      C₁-C₆ alkyl optionally substituted with 1-3 groups independently selected from hydroxyl and C₁-C₆ alkoxy,
   C₆-C₁₀ aryl,
   5- to 10-membered heteroaryl optionally substituted with 1-3 groups independently selected from C₁-C₆ alkyl, and
   3- to 10-membered heterocyclyl;
or two R^{N} on the same nitrogen atom are taken together with the nitrogen to which they are bonded to form a 3- to 10-membered heterocyclyl optionally substituted with 1-4 groups selected from:
   hydroxyl,
   halogen,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(R^{N})₂,
      C₁-C₉ alkyl optionally substituted with 1-4 groups independently selected from:
         oxo,
         halogen,
         hydroxyl,
         N(R^{N})₂,
         —SO₂-(C₁-C₆ alkyl),
         C₁-C₆ alkoxy optionally substituted with 1-3 groups independently selected from halogen and C₆-C₁₀ aryl,
         C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
         —O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₃-C₁₂ cycloalkyl optionally substituted with 1-4 groups independently selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy and C₁-C₆ fluoroalkyl, and
   5- to 12-membered heteroaryl optionally substituted with 1-3 groups independently selected from C₁-C₆ alkyl and C₁-C₆ fluoroalkyl.
      1a. A compound of Formula Ia:

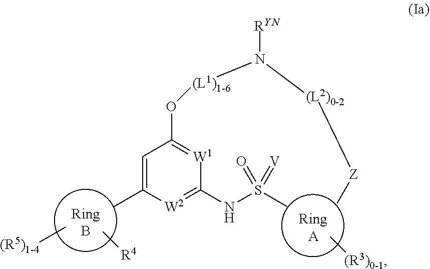

a tautomer thereof, a deuterated derivative of the compound or tautomer, or a pharmaceutically acceptable salt of any of the foregoing, wherein:
Ring A is selected from:
   C₆-C₁₀ aryl,
   C₃-C₁₀ cycloalkyl,
   3- to 10-membered heterocyclyl, and
   5- to 10-membered heteroaryl;
Ring B is selected from:
   C₆-C₁₀ aryl,
   C₃-C₁₀ cycloalkyl,
   3- to 10-membered heterocyclyl, and
   5- to 10-membered heteroaryl;
V is selected from O and NH;
W¹ is selected from N and CH;
W² is selected from N and CH; provided that at least one of W¹ and W² is N;
Z is selected from O, NR^{ZN}, and C(R^{ZC})₂, provided that when L² is absent, Z is C(R^{ZC})₂
each L¹ is independently selected from C(R^{L1})₂ and

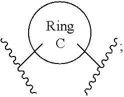

each L² is independently selected from C(R^{L2})₂;
Ring C is selected from C₆-C₁₀ aryl optionally substituted with 1-3 groups independently selected from:
   halogen,
   C₁-C₆ alkyl, and
   N(R^{N})₂;
each R³ is independently selected from:
   halogen,
   C₁-C₆ alkyl,
   C₁-C₆ alkoxy,
   C₃-C₁₀ cycloalkyl,
   C₆-C₁₀ aryl optionally substituted with 1-3 groups independently selected from C₁-C₆ alkyl, and
   3- to 10-membered heterocyclyl;
R⁴ is selected from hydrogen and C₁-C₆ alkyl;
each R⁵ is independently selected from:
   hydrogen,
   halogen,
   hydroxyl,
   N(R^{N})₂,
   —SO-Me,
   —CH═C(R^{LC})₂, wherein both R^{LC} are taken together to form a C₃-C₁₀ cycloalkyl,
   C₁-C₆ alkyl optionally substituted with 1-3 groups independently selected from:
      hydroxyl,
      C₁-C₆ alkoxy optionally substituted with 1-3 groups independently selected from C₁-C₆ alkoxy and C₆-C₁₀ aryl,
      C₃-C₁₀ cycloalkyl,
      —(O)₀₋₁—(C₆-C₁₀ aryl) optionally substituted with 1-3 groups independently selected from C₁-C₆ alkyl and C₁-C₆ alkoxy,
      3- to 10-membered heterocyclyl, and
      N(R^{N})₂,
   C₁-C₆ alkoxy optionally substituted with 1-3 groups independently selected from:
      halogen,
      C₆-C₁₀ aryl, and
      C₃-C₁₀ cycloalkyl optionally substituted with 1-3 groups independently selected from C₁-C₆ fluoroalkyl,
   C₁-C₆ fluoroalkyl,
   C₃-C₁₀ cycloalkyl,
   C₆-C₁₀ aryl, and
   3- to 10-membered heterocyclyl;
R^{YN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy,
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl (optionally substituted with 1-3 groups independently selected from oxo and C₁-C₆ alkoxy),
         C₁-C₆ alkoxy,
         C₁-C₆ fluoroalkyl,
         C₆-C₁₀ aryl optionally substituted with 1-3 groups independently selected from halogen,
         5- to 10-membered heteroaryl, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3-SiMe₃), and N(R^{N})₂,
         C₁-C₆ alkoxy optionally substituted with 1-3 groups independently selected from hydroxyl, oxo, halogen, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from halogen, C₁-C₆ alkyl, and C₁-C₆ alkoxy,
         —(O)₀₋₁—(C₆-C₁₀ aryl),
         —(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
         5- to 10-membered heteroaryl optionally substituted with 1-4 groups independently selected from C₁-C₆ alkyl and C₃-C₁₀ cycloalkyl, and
   C₁-C₆ fluoroalkyl;
R^{ZN} is selected from:
   hydrogen,
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         hydroxyl,
         C₁-C₆ alkyl optionally substituted with 1-3 groups independently selected from hydroxyl, oxo, C₁-C₆ alkoxy, C₆-C₁₀ aryl, and N(R^{N})₂,
         C₁-C₆ fluoroalkyl,
         C₁-C₆ alkoxy, and
         COOH,
         N(R^{N})₂,
         C₆-C₁₀ aryl, and
         3- to 10-membered heterocyclyl optionally substituted with 1-3 groups independently selected from oxo and C₁-C₆ alkyl,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl,
         —(O)₀₋₁—(C₆-C₁₀ aryl), and
         —(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
      3- to 10-membered heterocyclyl optionally substituted with 1-4 groups independently selected from:
         hydroxyl,
         oxo,
         N(R^{N})₂,
         C₁-C₆ alkyl (optionally substituted with 1-3 groups independently selected from oxo and C₁-C₆ alkoxy),
         C₁-C₆ alkoxy,
         C₁-C₆ fluoroalkyl,
         C₆-C₁₀ aryl optionally substituted with 1-3 groups independently selected from halogen,
         5- to 10-membered heteroaryl, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy (optionally substituted with 1-3-SiMe₃), and N(R^{N})₂,
         C₁-C₆ alkoxy optionally substituted with 1-3 groups independently selected from hydroxyl, oxo, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl) and
         5- to 10-membered heteroaryl optionally substituted with 1-4 groups independently selected from C₁-C₆ alkyl and C₃-C₁₀ cycloalkyl,
   C₁-C₆ fluoroalkyl,
   C₃-C₁₀ cycloalkyl optionally substituted with 1-3 groups independently selected from:
      hydroxyl,
      oxo,
      halogen,
      cyano,
      N(R^{N})₂,
      C₁-C₆ alkyl optionally substituted with 1-3 groups independently selected from:
         hydroxyl,
         oxo,
         N(R^{N})₂,
         C₁-C₆ alkoxy, and
         C₆-C₁₀ aryl,
      C₁-C₆ alkoxy optionally substituted with 1-3 groups independently selected from halogen, oxo, C₆-C₁₀ aryl, and N(R^{N})₂,
      halogen,
      C₃-C₁₀ cycloalkyl,
      3- to 10-member heterocyclyl optionally substituted with 1-3 groups independently selected from C₁-C₆ alkyl,
      5- to 10-membered heteroaryl optionally substituted with 1-3 groups independently selected from:
         hydroxyl,
         cyano,
         oxo,
         halogen,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         C₆-C₁₀ aryl,
         3- to 10-membered heterocyclyl optionally substituted with 1-3 groups independently selected from C₁-C₆ alkyl,
   C₆-C₁₀ aryl,
   3- to 10-membered heterocyclyl optionally substituted with 1-3 groups independently selected from:
      oxo,
      C₁-C₆ alkyl optionally substituted with 1-3 groups independently selected from:
         oxo,
         hydroxyl,
         N(R^{N})₂,
         C₁-C₆ alkoxy optionally substituted with 1-3 groups independently selected from halogen and C₆-C₁₀ aryl,
         —(O)₀₋₁—(C₃-C₁₀ cycloalkyl),
      C₁-C₆ fluoroalkyl,
      C₃-C₁₀ cycloalkyl optionally substituted with 1-3 groups independently selected from halogen, and
      3- to 10-membered heterocyclyl,
   5- to 10-membered heteroaryl optionally substituted with 1-3 groups independently selected from:
      halogen,
      C₁-C₆ alkyl optionally substituted with 1-3 groups independently selected from oxo, C₁-C₆ alkoxy, and N(R^{N})₂, and
      3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
   R^{F};
each R^{ZC} is independently selected from:
   hydrogen,
   C₁-C₆ alkyl optionally substituted with 1-3 groups independently selected from C₆-C₁₀ aryl (optionally substituted with 1-3 groups independently selected from C₁-C₆ alkyl),
   C₆-C₁₀ aryl optionally substituted with 1-3 groups independently selected from C₁-C₆ alkyl, and
   R^{F};
or two R^{ZC} are taken together to form an oxo group;
each R^{L1} is independently selected from:
   hydrogen,
   N(R^{N})₂, provided that two N(R^{N})₂ are not bonded to the same carbon,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from halogen and C₁-C₆ fluoroalkyl,
      C₆-C₁₀ aryl optionally substituted with 1-3 groups independently selected from C₁-C₆ alkyl, and
      3- to 10-membered heterocyclyl optionally substituted with 1-3 groups independently selected from C₁-C₆ alkyl (optionally substituted with 1-3 groups independently selected from hydroxyl and oxo),
   C₃-C₁₀ cycloalkyl,
   C₆-C₁₀ aryl optionally substituted with 1-4 groups independently selected from:
      halogen,
      cyano,
      SiMe₃,
      POMe₂,
      C₁-C₇ alkyl optionally substituted with 1-3 groups independently selected from:
         hydroxyl,
         oxo,
         cyano,
         SiMe₃,
         N(R^{N})₂, and
         C₃-C₁₀ cycloalkyl optionally substituted with 1-3 groups independently selected from C₁-C₆ fluoroalkyl,
      C₁-C₆ alkoxy optionally substituted with 1-3 groups independently selected from:
         C₃-C₁₀ cycloalkyl optionally substituted with 1-3 groups independently selected from C₁-C₆ fluoroalkyl, and
      C₁-C₆ alkoxy,
      C₁-C₆ fluoroalkyl,
      C₃-C₁₀ cycloalkyl optionally substituted with 1-3 groups independently selected from C₁-C₆ alkyl and C₁-C₆ fluoroalkyl,
      C₆-C₁₀ aryl,
      3- to 10-membered heterocyclyl optionally substituted with 1-3 groups independently selected from C₁-C₆ alkyl, and
      5- to 10-membered heteroaryl,
   3- to 10-membered heterocyclyl optionally substituted with 1-3 groups independently selected from:
      C₁-C₆ alkyl optionally substituted with 1-3 groups independently selected from:
         oxo, and
         C₁-C₆ alkoxy,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl, and
   R^{F}.
or two R^{L1} on the same carbon atom are taken together to form an oxo group;
each R^{L2} is independently selected from hydrogen and R^{F};
or two R^{L2} on the same carbon atom are taken together to form an oxo group;
each R^{N} is independently selected from:
   hydrogen,
   C₁-C₈ alkyl optionally substituted with 1-3 groups independently selected from:
      oxo,
      halogen,
      hydroxyl,
      NH₂,
      NHMe,
      NMe₂,
      NHCOMe,
      C₁-C₆ alkoxy optionally substituted with 1-3 groups independently selected from C₆-C₁₀ aryl,
      —(O)₀₋₁—(C₃-C₁₀ cycloalkyl),
      C₆-C₁₀ aryl optionally substituted with 1-3 groups independently selected from halogen and C₁-C₆ alkyl, and
      3- to 14-membered heterocyclyl optionally substituted with 1-4 groups independently selected from oxo and C₁-C₆ alkyl,
      5- to 14-membered heteroaryl optionally substituted with 1-4 groups independently selected from oxo and C₁-C₆ alkyl,
   C₃-C₁₀ cycloalkyl optionally substituted with 1-3 groups independently selected from:
      hydroxyl,
      NH₂, and
      NHMe,
      C₁-C₆ alkyl optionally substituted with 1-3 groups independently selected from hydroxyl, and
   C₆-C₁₀ aryl, and
   3- to 10-membered heterocyclyl;
or two R^{N} on the same nitrogen atom are taken together with the nitrogen to which they are bonded to form a 3- to 10-membered heterocyclyl optionally substituted with 1-4 groups selected from:
   hydroxyl,
   halogen,
   oxo,
   cyano,
   C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
   C₁-C₆ alkoxy, and
   C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
   C₃-C₁₀ cycloalkyl optionally substituted with 1-3 groups independently selected from C₁-C₆ alkyl,
   C₆-C₁₀ aryl optionally substituted with 1-3 groups independently selected from:
      halogen,
      C₁-C₆ alkyl,
      N(R^{N})₂, and
      3- to 10-membered heterocyclyl optionally substituted with 1-3 groups independently selected from hydroxyl,
   3- to 11-membered heterocyclyl optionally substituted with 1-3 groups independently selected from:
      oxo,
      N(R^{N})₂,
      C₁-C₉ alkyl optionally substituted with 1-4 groups independently selected from:
         oxo,
         halogen,
         hydroxyl,
         N(R^{N})₂,
         —SO₂-(C₁-C₆ alkyl),
         C₁-C₆ alkoxy optionally substituted with 1-3 groups independently selected from halogen, C₆-C₁₀ aryl,
         C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
         —(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
         3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
         —O-(5- to 12-membered heteroaryl) optionally substituted with 1-3 groups independently selected from C₆-C₁₀ aryl (optionally substituted with 1-3 groups independently selected from halogen) and C₁-C₆ alkyl, and
         5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), —O—(C₆-C₁₀ aryl), and C₃-C₁₀ cycloalkyl,
      C₃-C₁₂ cycloalkyl optionally substituted with 1-4 groups independently selected from halogen, C₁-C₆ alkyl, and C₁-C₆ fluoroalkyl,
      C₆-C₁₀ aryl,
      3- to 10-membered heterocyclyl, and
      5- to 10-membered heteroaryl optionally substituted with 1-3 groups independently selected from C₁-C₆ alkoxy and C₁-C₆ fluoroalkyl, and
   5- to 12-membered heteroaryl optionally substituted with 1-3 groups independently selected from C₁-C₆ alkyl and C₁-C₆ fluoroalkyl.
2. The compound, tautomer, deuterated derivative, or pharmaceutically acceptable salt according to embodiment 1, wherein Ring A is selected from selected from C₆-C₁₀ aryl and 5- to 10-membered heteroaryl.
3. The compound, tautomer, deuterated derivative, or pharmaceutically acceptable salt according to embodiment 1 or 2, wherein Ring A is selected from phenyl, pyridinyl, and pyrazolyl.
4. The compound, tautomer, deuterated derivative, or pharmaceutically acceptable salt according to any one of embodiments 1 to 3, wherein Ring A is phenyl.
5. The compound, tautomer, deuterated derivative, or pharmaceutically acceptable salt according to any one of embodiments 1 to 4, wherein Ring B is selected from C₆-C₁₀ aryl.
6. The compound, tautomer, deuterated derivative, or pharmaceutically acceptable salt according to any one of embodiments 1 to 5, wherein Ring B is selected from phenyl and naphthyl.
7. The compound, tautomer, deuterated derivative, or pharmaceutically acceptable salt according to any one of embodiments 1 to 6, wherein Ring B is phenyl.
8. The compound, tautomer, deuterated derivative, or pharmaceutically acceptable salt according to any one of embodiments 1 to 7, wherein V is O.
9. The compound, tautomer, deuterated derivative, or pharmaceutically acceptable salt according to any one of embodiments 1 to 8, wherein W¹ is N and W² is N.
10. The compound, tautomer, deuterated derivative, or pharmaceutically acceptable salt according to any one of embodiments 1 to 9, wherein Z is selected from NR^{ZN} and C(R^{ZC})₂, provided that when L² is absent, Z is C(R^{ZC})₂
11. The compound, tautomer, deuterated derivative, or pharmaceutically acceptable salt according to any one of embodiments 1 to 10, wherein each L¹ is C(R^{L1})₂
12. The compound, tautomer, deuterated derivative, or pharmaceutically acceptable salt according to any one of embodiments 1 to 11, wherein L² is absent.
13. The compound, tautomer, deuterated derivative, or pharmaceutically acceptable salt according to any one of embodiments 1 to 12, wherein each R³ is independently selected from C₁-C₆ alkyl.
14. The compound, tautomer, deuterated derivative, or pharmaceutically acceptable salt according to any one of embodiments 1 to 13, wherein each R³ is methyl.
15. The compound, tautomer, deuterated derivative, or pharmaceutically acceptable salt according to any one of embodiments 1 to 12, wherein R³ is absent.
16. The compound, tautomer, deuterated derivative, or pharmaceutically acceptable salt according to any one of embodiments 1 to 15, wherein R⁴ is selected from hydrogen and methyl.
17. The compound, tautomer, deuterated derivative, or pharmaceutically acceptable salt according to any one of embodiments 1 to 16, wherein R⁴ is methyl.
18. The compound, tautomer, deuterated derivative, or pharmaceutically acceptable salt according to any one of embodiments 1 to 17, wherein each R⁵ is independently selected from:
   hydrogen,
   hydroxyl,
   C₁-C₆ alkyl optionally substituted with 1-3 groups independently selected from C₃-C₁₀ cycloalkyl, and
   C₁-C₆ alkoxy optionally substituted with 1-3 groups independently selected from C₆-C₁₀ aryl.
19. The compound, tautomer, deuterated derivative, or pharmaceutically acceptable salt according to any one of embodiments 1 to 18, wherein R^{YN} is selected from:
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         C₁-C₆ alkyl optionally substituted with 1-3 groups independently selected from oxo and N(R^{N})₂,
         C₁-C₆ fluoroalkyl,
         C₁-C₆ alkoxy, and
         COOH,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl, and
         —(O)₀₋₁—(C₆-C₁₀ aryl),
      3- to 10-membered heterocyclyl optionally substituted with 1-4 groups independently selected from oxo and C₁-C₆ alkoxy, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from C₁-C₆ alkyl (optionally substituted with 1-3 groups independently selected from hydroxyl), and
         5- to 10-membered heteroaryl optionally substituted with 1-4 groups independently selected from C₁-C₆ alkyl.
20. The compound, tautomer, deuterated derivative, or pharmaceutically acceptable salt according to any one of embodiments 1 to 19, wherein each R^{ZC} is hydrogen, or two R^{ZC} are taken together to form an oxo group.
21. The compound, tautomer, deuterated derivative, or pharmaceutically acceptable salt according to any one of embodiments 1 to 20, wherein each R^{L1} is independently selected from:
   hydrogen,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from C₁-C₆ fluoroalkyl, and
      3- to 10-membered heterocyclyl optionally substituted with 1-3 groups independently selected from C₁-C₆ alkyl (optionally substituted with 1-3 groups independently selected from hydroxyl and oxo),
   C₆-C₁₀ aryl optionally substituted with 1-4 groups independently selected from:
      halogen,
      cyano,
      C₁-C₇ alkyl optionally substituted with 1-3 groups independently selected from oxo and N(R^{N})₂,
      C₃-C₁₀ cycloalkyl,
      3- to 10-membered heterocyclyl optionally substituted with 1-3 groups independently selected from C₁-C₆ alkyl, and
      5- to 10-membered heteroaryl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl.
22. The compound, tautomer, deuterated derivative, or pharmaceutically acceptable salt according to any one of embodiments 1 to 21, wherein each C(R^{L2})₂ is CH₂ or C═O.
23. The compound, tautomer, deuterated derivative, or pharmaceutically acceptable salt according to any one of embodiments 1 to 22, wherein each R^{N} is independently selected from:
   hydrogen,
   C₁-C₈ alkyl optionally substituted with 1-3 groups independently selected from:
      oxo,
      hydroxyl,
      C₁-C₆ alkoxy,
      —(O)₀₋₁—(C₃-C₁₀ cycloalkyl),
      C₆-C₁₀ aryl,
      3- to 14-membered heterocyclyl,
      5- to 14-membered heteroaryl optionally substituted with 1-4 groups independently selected from C₁-C₆ alkyl,
   C₃-C₁₀ cycloalkyl optionally substituted with 1-3 groups independently selected from C₁-C₆ alkyl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from C₁-C₆ alkyl.
24. The compound, tautomer, deuterated derivative, or pharmaceutically acceptable salt according to any one of embodiments 1 to 23, wherein when R^{F} is present, two R^{F} taken together with the atoms to which they are bonded form a group selected from 3- to 11-membered heterocyclyl optionally substituted with 1-3 groups independently selected from:
   C₁-C₉ alkyl, and
   5- to 10-membered heteroaryl optionally substituted with 1-3 groups independently selected from C₁-C₆ alkoxy.
25. A compound of Formula Ib:

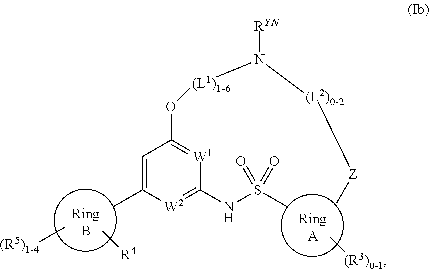

a tautomer thereof, a deuterated derivative of the compound or tautomer, or a pharmaceutically acceptable salt of any of the foregoing, wherein Ring A, Ring B, W¹, W², Z, L¹, L², R³, R⁴, R⁵, and R^{YN} are defined as according to embodiment 1a.
26. The compound, tautomer, deuterated derivative, or pharmaceutically acceptable salt according to embodiment 25, wherein Ring A is selected from selected from C₆-C₁₀ aryl and 5- to 10-membered heteroaryl.
27. The compound, tautomer, deuterated derivative, or pharmaceutically acceptable salt according to embodiment 25 or 26, wherein Ring A is selected from phenyl, pyridinyl, and pyrazolyl.
28. The compound, tautomer, deuterated derivative, or pharmaceutically acceptable salt according to any one of embodiments 25 to 27, wherein Ring A is phenyl.
29. The compound, tautomer, deuterated derivative, or pharmaceutically acceptable salt according to any one of embodiments 25 to 28, wherein Ring B is selected from C₆-C₁₀ aryl.
30. The compound, tautomer, deuterated derivative, or pharmaceutically acceptable salt according to any one of embodiments 25 to 29, wherein Ring B is selected from phenyl and naphthyl.
31. The compound, tautomer, deuterated derivative, or pharmaceutically acceptable salt according to any one of embodiments 25 to 30, wherein Ring B is phenyl.
32. The compound, tautomer, deuterated derivative, or pharmaceutically acceptable salt according to any one of embodiments 25 to 31, wherein W¹ is N and W² is N.
33. The compound, tautomer, deuterated derivative, or pharmaceutically acceptable salt according to any one of embodiments 25 to 32, wherein Z is selected from NR^{ZN} and C(R^{ZC})₂, provided that when L² is absent, Z is C(R^{ZC})₂
34. The compound, tautomer, deuterated derivative, or pharmaceutically acceptable salt according to any one of embodiments 25 to 33, wherein each L¹ is C(R^{L1})₂.
35. The compound, tautomer, deuterated derivative, or pharmaceutically acceptable salt according to any one of embodiments 25 to 34, wherein L² is absent.
36. The compound, tautomer, deuterated derivative, or pharmaceutically acceptable salt according to any one of embodiments 25 to 35, wherein each R³ is independently selected from C₁-C₆ alkyl.
37. The compound, tautomer, deuterated derivative, or pharmaceutically acceptable salt according to any one of embodiments 25 to 36, wherein each R³ is methyl.
38. The compound, tautomer, deuterated derivative, or pharmaceutically acceptable salt according to any one of embodiments 25 to 35, wherein R³ is absent.
39. The compound, tautomer, deuterated derivative, or pharmaceutically acceptable salt according to any one of embodiments 25 to 38, wherein R⁴ is selected from hydrogen and methyl.
40. The compound, tautomer, deuterated derivative, or pharmaceutically acceptable salt according to any one of embodiments 25 to 39, wherein R⁴ is methyl.
41. The compound, tautomer, deuterated derivative, or pharmaceutically acceptable salt according to any one of embodiments 25 to 40, wherein each R⁵ is independently selected from:
   hydrogen,
   hydroxyl,
   C₁-C₆ alkyl optionally substituted with 1-3 groups independently selected from C₃-C₁₀ cycloalkyl,
   C₁-C₆ alkoxy optionally substituted with 1-3 groups independently selected from C₆-C₁₀ aryl,
42. The compound, tautomer, deuterated derivative, or pharmaceutically acceptable salt according to any one of embodiments 25 to 41, wherein R^{YN} is selected from:
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         C₁-C₆ alkyl optionally substituted with 1-3 groups independently selected from oxo and N(R^{N})₂,
         C₁-C₆ fluoroalkyl,
         C₁-C₆ alkoxy, and
         COOH,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl, and
         —(O)₀₋₁—(C₆-C₁₀ aryl),
      3- to 10-membered heterocyclyl optionally substituted with 1-4 groups independently selected from oxo and C₁-C₆ alkoxy, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from C₁-C₆ alkyl (optionally substituted with 1-3 groups independently selected from hydroxyl), and
         5- to 10-membered heteroaryl optionally substituted with 1-4 groups independently selected from C₁-C₆ alkyl.
43. The compound, tautomer, deuterated derivative, or pharmaceutically acceptable salt according to any one of embodiments 25 to 42, wherein each R^{ZC} is hydrogen, or two R^{ZC} are taken together to form an oxo group;
44. The compound, tautomer, deuterated derivative, or pharmaceutically acceptable salt according to any one of embodiments 25 to 43, wherein each R^{L1} is independently selected from:
   hydrogen,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from C₁-C₆ fluoroalkyl, and
      3- to 10-membered heterocyclyl optionally substituted with 1-3 groups independently selected from C₁-C₆ alkyl (optionally substituted with 1-3 groups independently selected from hydroxyl and oxo),
   C₆-C₁₀ aryl optionally substituted with 1-4 groups independently selected from:
      halogen,
      cyano,
      C₁-C₇ alkyl optionally substituted with 1-3 groups independently selected from oxo and N(R^{N})₂,
      C₃-C₁₀ cycloalkyl,
      3- to 10-membered heterocyclyl optionally substituted with 1-3 groups independently selected from C₁-C₆ alkyl, and
      5- to 10-membered heteroaryl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl.
45. The compound, tautomer, deuterated derivative, or pharmaceutically acceptable salt according to any one of embodiments 25 to 44, wherein each C(R^{L2})₂ is CH₂ or C═O.
46. The compound, tautomer, deuterated derivative, or pharmaceutically acceptable salt according to any one of embodiments 25 to 45, wherein each R^{N} is independently selected from:
   hydrogen,
   C₁-C₈ alkyl optionally substituted with 1-3 groups independently selected from:
      oxo,
      hydroxyl,
      C₁-C₆ alkoxy,
      —(O)₀₋₁—(C₃-C₁₀ cycloalkyl),
      C₆-C₁₀ aryl,
      3- to 14-membered heterocyclyl,
      5- to 14-membered heteroaryl optionally substituted with 1-4 groups independently selected from C₁-C₆ alkyl,
   C₃-C₁₀ cycloalkyl optionally substituted with 1-3 groups independently selected from C₁-C₆ alkyl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from C₁-C₆ alkyl.
47. The compound, tautomer, deuterated derivative, or pharmaceutically acceptable salt according to any one of embodiments 25 to 46, wherein when R^{F} is present, two R^{F}taken together with the atoms to which they are bonded form a group selected from 3- to 11-membered heterocyclyl optionally substituted with 1-3 groups independently selected from:
   C₁-C₉ alkyl, and
   5- to 10-membered heteroaryl optionally substituted with 1-3 groups independently selected from C₁-C₆ alkoxy.
48. A compound of Formula IIa:

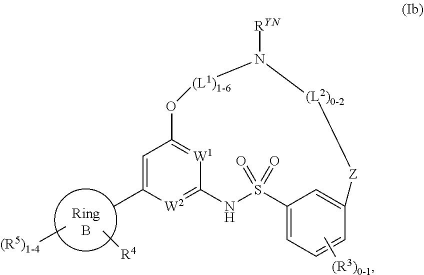

a tautomer thereof, a deuterated derivative of the compound or tautomer, or a pharmaceutically acceptable salt of any of the foregoing, wherein Ring B, W¹, W², Z, L¹, L², R³, R⁴, R⁵, and R^{YN} are defined as according to embodiment 1a.
49. The compound, tautomer, deuterated derivative, or pharmaceutically acceptable salt according to embodiment 48, wherein Ring B is selected from C₆-C₁₀ aryl.
50. The compound, tautomer, deuterated derivative, or pharmaceutically acceptable salt according to embodiment 48 or 47, wherein Ring B is selected from phenyl and naphthyl.
51. The compound, tautomer, deuterated derivative, or pharmaceutically acceptable salt according to any one of embodiments 48 to 50, wherein Ring B is phenyl.
52. The compound, tautomer, deuterated derivative, or pharmaceutically acceptable salt according to any one of embodiments 48 to 51, wherein W¹ is N and W² is N.
53. The compound, tautomer, deuterated derivative, or pharmaceutically acceptable salt according to any one of embodiments 48 to 52, wherein Z is selected from NR^{ZN} and C(R^{ZC})₂, provided that when L² is absent, Z is C(R^{ZC})₂
54. The compound, tautomer, deuterated derivative, or pharmaceutically acceptable salt according to any one of embodiments 48 to 53, wherein each L¹ is C(R^{L1})₂.
55. The compound, tautomer, deuterated derivative, or pharmaceutically acceptable salt according to any one of embodiments 48 to 54, wherein L² is absent.
56. The compound, tautomer, deuterated derivative, or pharmaceutically acceptable salt according to any one of embodiments 48 to 55, wherein each R³ is independently selected from C₁-C₆ alkyl.
57. The compound, tautomer, deuterated derivative, or pharmaceutically acceptable salt according to any one of embodiments 48 to 56, wherein each R³ is methyl.
58. The compound, tautomer, deuterated derivative, or pharmaceutically acceptable salt according to any one of embodiments 48 to 55, wherein R³ is absent.
59. The compound, tautomer, deuterated derivative, or pharmaceutically acceptable salt according to any one of embodiments 48 to 58, wherein R⁴ is selected from hydrogen and methyl.
60. The compound, tautomer, deuterated derivative, or pharmaceutically acceptable salt according to any one of embodiments 48 to 59, wherein R⁴ is methyl.
61. The compound, tautomer, deuterated derivative, or pharmaceutically acceptable salt according to any one of embodiments 48 to 60, wherein each R⁵ is independently selected from:
   hydrogen,
   hydroxyl,
   C₁-C₆ alkyl optionally substituted with 1-3 groups independently selected from C₃-C₁₀ cycloalkyl,
   C₁-C₆ alkoxy optionally substituted with 1-3 groups independently selected from C₆-C₁₀ aryl,
62. The compound, tautomer, deuterated derivative, or pharmaceutically acceptable salt according to any one of embodiments 48 to 61, wherein R^{YN} is selected from:
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         C₁-C₆ alkyl optionally substituted with 1-3 groups independently selected from oxo and N(R^{N})₂,
         C₁-C₆ fluoroalkyl,
         C₁-C₆ alkoxy, and
         COOH,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl, and
         —(O)₀₋₁—(C₆-C₁₀ aryl),
      3- to 10-membered heterocyclyl optionally substituted with 1-4 groups independently selected from oxo and C₁-C₆ alkoxy, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from C₁-C₆ alkyl (optionally substituted with 1-3 groups independently selected from hydroxyl), and
         5- to 10-membered heteroaryl optionally substituted with 1-4 groups independently selected from C₁-C₆ alkyl.
63. The compound, tautomer, deuterated derivative, or pharmaceutically acceptable salt according to any one of embodiments 48 to 62, wherein each R^{ZC} is hydrogen, or two R^{ZC} are taken together to form an oxo group;
64. The compound, tautomer, deuterated derivative, or pharmaceutically acceptable salt according to any one of embodiments 48 to 63, wherein each R^{L1} is independently selected from:
   hydrogen,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from C₁-C₆ fluoroalkyl, and
      3- to 10-membered heterocyclyl optionally substituted with 1-3 groups independently selected from C₁-C₆ alkyl (optionally substituted with 1-3 groups independently selected from hydroxyl and oxo),
   C₆-C₁₀ aryl optionally substituted with 1-4 groups independently selected from:
      halogen,
      cyano,
      C₁-C₇ alkyl optionally substituted with 1-3 groups independently selected from oxo and N(R^{N})₂,
      C₃-C₁₀ cycloalkyl,
      3- to 10-membered heterocyclyl optionally substituted with 1-3 groups independently selected from C₁-C₆ alkyl, and
      5- to 10-membered heteroaryl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl.
65. The compound, tautomer, deuterated derivative, or pharmaceutically acceptable salt according to any one of embodiments 48 to 64, wherein each C(R^{L2})₂ is CH₂ or C═O.
66. The compound, tautomer, deuterated derivative, or pharmaceutically acceptable salt according to any one of embodiments 48 to 65, wherein each R^{N} is independently selected from:
   hydrogen,
   C₁-C₈ alkyl optionally substituted with 1-3 groups independently selected from:
      oxo,
      hydroxyl,
      C₁-C₆ alkoxy,
      —(O)₀₋₁—(C₃-C₁₀ cycloalkyl),
      C₆-C₁₀ aryl,
      3- to 14-membered heterocyclyl,
      5- to 14-membered heteroaryl optionally substituted with 1-4 groups independently selected from C₁-C₆ alkyl,
   C₃-C₁₀ cycloalkyl optionally substituted with 1-3 groups independently selected from C₁-C₆ alkyl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from C₁-C₆ alkyl.
67. The compound, tautomer, deuterated derivative, or pharmaceutically acceptable salt according to any one of embodiments 48 to 66, wherein when R^{F} is present, two R^{F}taken together with the atoms to which they are bonded form a group selected from 3- to 11-membered heterocyclyl optionally substituted with 1-3 groups independently selected from:
   C₁-C₉ alkyl, and
   5- to 10-membered heteroaryl optionally substituted with 1-3 groups independently selected from C₁-C₆ alkoxy.
68. A compound of Formula IIb:

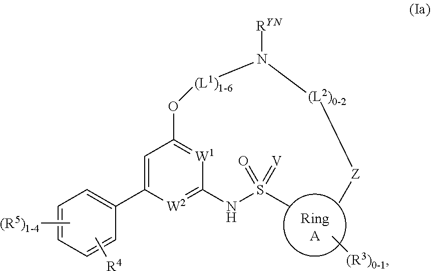

a tautomer thereof, a deuterated derivative of the compound or tautomer, or a pharmaceutically acceptable salt of any of the foregoing, wherein Ring A, W¹, W², Z, L¹, L², R³, R⁴, R⁵, and R^{YN} are defined as according to embodiment 1a.
69. The compound, tautomer, deuterated derivative, or pharmaceutically acceptable salt according to embodiment 68, wherein Ring A is selected from selected from C₆-C₁₀ aryl and 5- to 10-membered heteroaryl.
70. The compound, tautomer, deuterated derivative, or pharmaceutically acceptable salt according to embodiment 68 or 69, wherein Ring A is selected from phenyl, pyridinyl, and pyrazolyl.
71. The compound, tautomer, deuterated derivative, or pharmaceutically acceptable salt according to any one of embodiments 68 to 70, wherein Ring A is phenyl.
72. The compound, tautomer, deuterated derivative, or pharmaceutically acceptable salt according to any one of embodiments 68 to 71, wherein W¹ is N and W² is N.
73. The compound, tautomer, deuterated derivative, or pharmaceutically acceptable salt according to any one of embodiments 68 to 72, wherein Z is selected from NR^{ZN} and C(R^{ZC})₂, provided that when L² is absent, Z is C(R^{ZC})₂
74. The compound, tautomer, deuterated derivative, or pharmaceutically acceptable salt according to any one of embodiments 68 to 73, wherein each L¹ is C(R^{L1})₂.
75. The compound, tautomer, deuterated derivative, or pharmaceutically acceptable salt according to any one of embodiments 68 to 74, wherein L² is absent.
76. The compound, tautomer, deuterated derivative, or pharmaceutically acceptable salt according to any one of embodiments 68 to 75, wherein each R³ is independently selected from C₁-C₆ alkyl.
77. The compound, tautomer, deuterated derivative, or pharmaceutically acceptable salt according to any one of embodiments 68 to 76, wherein each R³ is methyl.
78. The compound, tautomer, deuterated derivative, or pharmaceutically acceptable salt according to any one of embodiments 68 to 75, wherein R³ is absent.
79. The compound, tautomer, deuterated derivative, or pharmaceutically acceptable salt according to any one of embodiments 68 to 78, wherein R⁴ is selected from hydrogen and methyl.
80. The compound, tautomer, deuterated derivative, or pharmaceutically acceptable salt according to any one of embodiments 68 to 79, wherein R⁴ is methyl.
81. The compound, tautomer, deuterated derivative, or pharmaceutically acceptable salt according to any one of embodiments 68 to 80, wherein each R⁵ is independently selected from:
   hydrogen,
   hydroxyl,
   C₁-C₆ alkyl optionally substituted with 1-3 groups independently selected from C₃-C₁₀ cycloalkyl,
   C₁-C₆ alkoxy optionally substituted with 1-3 groups independently selected from C₆-C₁₀ aryl,
82. The compound, tautomer, deuterated derivative, or pharmaceutically acceptable salt according to any one of embodiments 68 to 81, wherein R^{YN} is selected from:
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         C₁-C₆ alkyl optionally substituted with 1-3 groups independently selected from oxo and N(R^{N})₂,
         C₁-C₆ fluoroalkyl,
         C₁-C₆ alkoxy, and
         COOH,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl, and
         —(O)₀₋₁—(C₆-C₁₀ aryl),
      3- to 10-membered heterocyclyl optionally substituted with 1-4 groups independently selected from oxo and C₁-C₆ alkoxy, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from C₁-C₆ alkyl (optionally substituted with 1-3 groups independently selected from hydroxyl), and
         5- to 10-membered heteroaryl optionally substituted with 1-4 groups independently selected from C₁-C₆ alkyl.
83. The compound, tautomer, deuterated derivative, or pharmaceutically acceptable salt according to any one of embodiments 68 to 82, wherein each R^{ZC} is hydrogen, or two R^{ZC} are taken together to form an oxo group;
84. The compound, tautomer, deuterated derivative, or pharmaceutically acceptable salt according to any one of embodiments 68 to 83, wherein each R^{L1} is independently selected from:
   hydrogen,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from C₁-C₆ fluoroalkyl, and
      3- to 10-membered heterocyclyl optionally substituted with 1-3 groups independently selected from C₁-C₆ alkyl (optionally substituted with 1-3 groups independently selected from hydroxyl and oxo),
   C₆-C₁₀ aryl optionally substituted with 1-4 groups independently selected from:
      halogen,
      cyano,
      C₁-C₇ alkyl optionally substituted with 1-3 groups independently selected from oxo and N(R^{N})₂,
      C₃-C₁₀ cycloalkyl,
      3- to 10-membered heterocyclyl optionally substituted with 1-3 groups independently selected from C₁-C₆ alkyl, and
      5- to 10-membered heteroaryl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl.
85. The compound, tautomer, deuterated derivative, or pharmaceutically acceptable salt according to any one of embodiments 68 to 84, wherein each C(R^{L2})₂ is CH₂ or C═O.
86. The compound, tautomer, deuterated derivative, or pharmaceutically acceptable salt according to any one of embodiments 68 to 85, wherein each R^{N} is independently selected from:
   hydrogen,
   C₁-C₈ alkyl optionally substituted with 1-3 groups independently selected from:
      oxo,
      hydroxyl,
      C₁-C₆ alkoxy,
      —(O)₀₋₁—(C₃-C₁₀ cycloalkyl),
      C₆-C₁₀ aryl,
      3- to 14-membered heterocyclyl,
      5- to 14-membered heteroaryl optionally substituted with 1-4 groups independently selected from C₁-C₆ alkyl,
   C₃-C₁₀ cycloalkyl optionally substituted with 1-3 groups independently selected from C₁-C₆ alkyl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from C₁-C₆ alkyl.
87. The compound, tautomer, deuterated derivative, or pharmaceutically acceptable salt according to any one of embodiments 68 to 86, wherein when R^{F} is present, two R^{F}taken together with the atoms to which they are bonded form a group selected from 3- to 11-membered heterocyclyl optionally substituted with 1-3 groups independently selected from:
   C₁-C₉ alkyl, and
   5- to 10-membered heteroaryl optionally substituted with 1-3 groups independently selected from C₁-C₆ alkoxy.
88. A compound of Formula III:

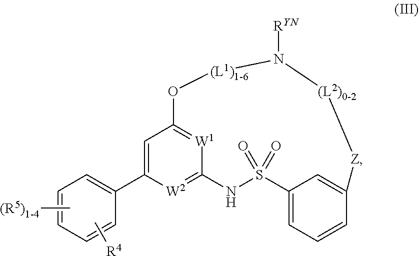

a tautomer thereof, a deuterated derivative of the compound or tautomer, or a pharmaceutically acceptable salt of any of the foregoing, wherein W¹, W², Z, L¹, L², R⁴, R⁵, and R^{YN} are defined as according to embodiment 1a.
89. The compound, tautomer, deuterated derivative, or pharmaceutically acceptable salt according to embodiment 88, wherein W¹ is N and W² is N.
90. The compound, tautomer, deuterated derivative, or pharmaceutically acceptable salt according to embodiment 88 or 89, wherein Z is selected from NR^{ZN} and C(R^{ZC}), provided that when L² is absent, Z is C(R^{ZC}).
91. The compound, tautomer, deuterated derivative, or pharmaceutically acceptable salt according to any one of embodiments 88 to 90, wherein each L¹ is C(R^{L})₂.
92. The compound, tautomer, deuterated derivative, or pharmaceutically acceptable salt according to any one of embodiments 88 to 91, wherein L² is absent.
93. The compound, tautomer, deuterated derivative, or pharmaceutically acceptable salt according to any one of embodiments 88 to 92, wherein R⁴ is selected from hydrogen and methyl.
94. The compound, tautomer, deuterated derivative, or pharmaceutically acceptable salt according to any one of embodiments 88 to 93, wherein R⁴ is methyl.
95. The compound, tautomer, deuterated derivative, or pharmaceutically acceptable salt according to any one of embodiments 88 to 94, wherein each R⁵ is independently selected from:
   hydrogen,
   hydroxyl,
   C₁-C₆ alkyl optionally substituted with 1-3 groups independently selected from C₃-C₁₀ cycloalkyl,
   C₁-C₆ alkoxy optionally substituted with 1-3 groups independently selected from C₆-C₁₀ aryl,
96. The compound, tautomer, deuterated derivative, or pharmaceutically acceptable salt according to any one of embodiments 88 to 95, wherein R^{YN} is selected from:
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         C₁-C₆ alkyl optionally substituted with 1-3 groups independently selected from oxo and N(R^{N})₂,
         C₁-C₆ fluoroalkyl,
         C₁-C₆ alkoxy, and
         COOH,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl, and
         —(O)₀₋₁—(C₆-C₁₀ aryl),
      3- to 10-membered heterocyclyl optionally substituted with 1-4 groups independently selected from oxo and C₁-C₆ alkoxy, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from C₁-C₆ alkyl (optionally substituted with 1-3 groups independently selected from hydroxyl), and
         5- to 10-membered heteroaryl optionally substituted with 1-4 groups independently selected from C₁-C₆ alkyl.
97. The compound, tautomer, deuterated derivative, or pharmaceutically acceptable salt according to any one of embodiments 88 to 96, wherein each R^{ZC} is hydrogen, or two R^{ZC} are taken together to form an oxo group;
98. The compound, tautomer, deuterated derivative, or pharmaceutically acceptable salt according to any one of embodiments 88 to 97, wherein each R^{L1} is independently selected from:
   hydrogen,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from C₁-C₆ fluoroalkyl, and
      3- to 10-membered heterocyclyl optionally substituted with 1-3 groups independently selected from C₁-C₆ alkyl (optionally substituted with 1-3 groups independently selected from hydroxyl and oxo),
   C₆-C₁₀ aryl optionally substituted with 1-4 groups independently selected from:
      halogen,
      cyano,
      C₁-C₇ alkyl optionally substituted with 1-3 groups independently selected from oxo and N(R^{N})₂,
      C₃-C₁₀ cycloalkyl,
      3- to 10-membered heterocyclyl optionally substituted with 1-3 groups independently selected from C₁-C₆ alkyl, and
      5- to 10-membered heteroaryl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl.
99. The compound, tautomer, deuterated derivative, or pharmaceutically acceptable salt according to any one of embodiments 88 to 98, wherein each C(R^{L2})₂ is CH₂ or C═O.
100. The compound, tautomer, deuterated derivative, or pharmaceutically acceptable salt according to any one of embodiments 88 to 99, wherein each R^{N} is independently selected from:
   hydrogen,
   C₁-C₈ alkyl optionally substituted with 1-3 groups independently selected from:
      oxo,
      hydroxyl,
      C₁-C₆ alkoxy,
      —(O)₀₋₁—(C₃-C₁₀ cycloalkyl),
      C₆-C₁₀ aryl,
      3- to 14-membered heterocyclyl,
      5- to 14-membered heteroaryl optionally substituted with 1-4 groups independently selected from C₁-C₆ alkyl,
   C₃-C₁₀ cycloalkyl optionally substituted with 1-3 groups independently selected from C₁-C₆ alkyl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from C₁-C₆ alkyl.
101. The compound, tautomer, deuterated derivative, or pharmaceutically acceptable salt according to any one of embodiments 88 to 100, wherein when R^{F} is present, two R^{F}taken together with the atoms to which they are bonded form a group selected from 3- to 11-membered heterocyclyl optionally substituted with 1-3 groups independently selected from:
   C₁-C₉ alkyl, and
   5- to 10-membered heteroaryl optionally substituted with 1-3 groups independently selected from C₁-C₆ alkoxy.
102. A compound of Formula IV:

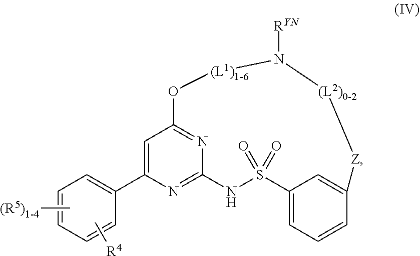

a tautomer thereof, a deuterated derivative of the compound or tautomer, or a pharmaceutically acceptable salt of any of the foregoing, wherein Z, L¹, L², R⁴, R⁵, and R^{YN} are defined as according to embodiment 1a.
103. The compound, tautomer, deuterated derivative, or pharmaceutically acceptable salt according to embodiment 102, wherein Z is selected from NR^{ZN} and C(R^{ZC})₂, provided that when L² is absent, Z is C(R^{ZC})₂.
104. The compound, tautomer, deuterated derivative, or pharmaceutically acceptable salt according to embodiment 102 or 103, wherein each L¹ is C(R^{L1})₂.
105. The compound, tautomer, deuterated derivative, or pharmaceutically acceptable salt according to any one of embodiments 102 to 104, wherein L² is absent.
106. The compound, tautomer, deuterated derivative, or pharmaceutically acceptable salt according to any one of embodiments 102 to 105, wherein R⁴ is selected from hydrogen and methyl.
107. The compound, tautomer, deuterated derivative, or pharmaceutically acceptable salt according to any one of embodiments 102 to 106, wherein R⁴ is methyl.
108. The compound, tautomer, deuterated derivative, or pharmaceutically acceptable salt according to any one of embodiments 102 to 107, wherein each R⁵ is independently selected from:
   hydrogen,
   hydroxyl,
   C₁-C₆ alkyl optionally substituted with 1-3 groups independently selected from C₃-C₁₀ cycloalkyl,
   C₁-C₆ alkoxy optionally substituted with 1-3 groups independently selected from C₆-C₁₀ aryl,
109. The compound, tautomer, deuterated derivative, or pharmaceutically acceptable salt according to any one of embodiments 102 to 108, wherein R^{YN} is selected from:
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         C₁-C₆ alkyl optionally substituted with 1-3 groups independently selected from oxo and N(R^{N})₂,
         C₁-C₆ fluoroalkyl,
         C₁-C₆ alkoxy, and
         COOH,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl, and
         —(O)₀₋₁—(C₆-C₁₀ aryl),
      3- to 10-membered heterocyclyl optionally substituted with 1-4 groups independently selected from oxo and C₁-C₆ alkoxy, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from C₁-C₆ alkyl (optionally substituted with 1-3 groups independently selected from hydroxyl), and
         5- to 10-membered heteroaryl optionally substituted with 1-4 groups independently selected from C₁-C₆ alkyl.
110. The compound, tautomer, deuterated derivative, or pharmaceutically acceptable salt according to any one of embodiments 102 to 109, wherein each R^{ZC} is hydrogen, or two R^{ZC} are taken together to form an oxo group;
111. The compound, tautomer, deuterated derivative, or pharmaceutically acceptable salt according to any one of embodiments 102 to 110, wherein each R^{L1} is independently selected from:
   hydrogen,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from C₁-C₆ fluoroalkyl, and
      3- to 10-membered heterocyclyl optionally substituted with 1-3 groups independently selected from C₁-C₆ alkyl (optionally substituted with 1-3 groups independently selected from hydroxyl and oxo),
   C₆-C₁₀ aryl optionally substituted with 1-4 groups independently selected from:
      halogen,
      cyano,
      C₁-C₇ alkyl optionally substituted with 1-3 groups independently selected from oxo and N(R^{N})₂,
      C₃-C₁₀ cycloalkyl,
      3- to 10-membered heterocyclyl optionally substituted with 1-3 groups independently selected from C₁-C₆ alkyl, and
      5- to 10-membered heteroaryl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl.
112. The compound, tautomer, deuterated derivative, or pharmaceutically acceptable salt according to any one of embodiments 102 to 111, wherein each C(R^{L2})₂ is CH₂ or C═O.
113. The compound, tautomer, deuterated derivative, or pharmaceutically acceptable salt according to any one of embodiments 102 to 112, wherein each R^{N} is independently selected from:
   hydrogen,
   C₁-C₈ alkyl optionally substituted with 1-3 groups independently selected from:
      oxo,
      hydroxyl,
      C₁-C₆ alkoxy,
      —(O)₀₋₁—(C₃-C₁₀ cycloalkyl),
      C₆-C₁₀ aryl,
      3- to 14-membered heterocyclyl,
      5- to 14-membered heteroaryl optionally substituted with 1-4 groups independently selected from C₁-C₆ alkyl,
   C₃-C₁₀ cycloalkyl optionally substituted with 1-3 groups independently selected from C₁-C₆ alkyl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from C₁-C₆ alkyl.
114. The compound, tautomer, deuterated derivative, or pharmaceutically acceptable salt according to any one of embodiments 102 to 113, wherein when R^{F} is present, two R^{F}taken together with the atoms to which they are bonded form a group selected from 3- to 11-membered heterocyclyl optionally substituted with 1-3 groups independently selected from:
   C₁-C₉ alkyl, and
   5- to 10-membered heteroaryl optionally substituted with 1-3 groups independently selected from C₁-C₆ alkoxy.
115. A compound of Formula V:

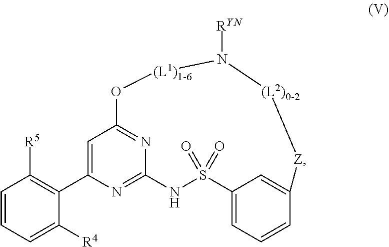

a tautomer thereof, a deuterated derivative of the compound or tautomer, or a pharmaceutically acceptable salt of any of the foregoing, wherein Z, L¹, L², R⁴, R⁵, and R^{YN} are defined as according to embodiment 1a.
116. The compound, tautomer, deuterated derivative, or pharmaceutically acceptable salt according to embodiment 115, wherein Z is selected from NR^{ZN} and C(R^{ZC})₂, provided that when L² is absent, Z is C(R^{ZC})₂.
117. The compound, tautomer, deuterated derivative, or pharmaceutically acceptable salt according to embodiment 115 or 116, wherein each L¹ is C(R^{L1})₂.
118. The compound, tautomer, deuterated derivative, or pharmaceutically acceptable salt according to any one of embodiments 115 to 117, wherein L² is absent.
119. The compound, tautomer, deuterated derivative, or pharmaceutically acceptable salt according to any one of embodiments 115 to 118, wherein R⁴ is selected from hydrogen and methyl.
120. The compound, tautomer, deuterated derivative, or pharmaceutically acceptable salt according to any one of embodiments 115 to 119, wherein R⁴ is methyl.
121. The compound, tautomer, deuterated derivative, or pharmaceutically acceptable salt according to any one of embodiments 115 to 120, wherein each R⁵ is independently selected from:
   hydrogen,
   hydroxyl,
   C₁-C₆ alkyl optionally substituted with 1-3 groups independently selected from C₃-C₁₀ cycloalkyl,
   C₁-C₆ alkoxy optionally substituted with 1-3 groups independently selected from C₆-C₁₀ aryl,
122. The compound, tautomer, deuterated derivative, or pharmaceutically acceptable salt according to any one of embodiments 115 to 121, wherein R^{YN} is selected from:
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         C₁-C₆ alkyl optionally substituted with 1-3 groups independently selected from oxo and N(R^{N})₂,
         C₁-C₆ fluoroalkyl,
         C₁-C₆ alkoxy, and
         COOH,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl, and
         —(O)₀₋₁—(C₆-C₁₀ aryl),
      3- to 10-membered heterocyclyl optionally substituted with 1-4 groups independently selected from oxo and C₁-C₆ alkoxy, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from C₁-C₆ alkyl (optionally substituted with 1-3 groups independently selected from hydroxyl), and
         5- to 10-membered heteroaryl optionally substituted with 1-4 groups independently selected from C₁-C₆ alkyl.
123. The compound, tautomer, deuterated derivative, or pharmaceutically acceptable salt according to any one of embodiments 115 to 122, wherein each R^{ZC} is hydrogen, or two R^{ZC} are taken together to form an oxo group;
124. The compound, tautomer, deuterated derivative, or pharmaceutically acceptable salt according to any one of embodiments 115 to 123, wherein each R^{L1} is independently selected from:
   hydrogen,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from C₁-C₆ fluoroalkyl, and
      3- to 10-membered heterocyclyl optionally substituted with 1-3 groups independently selected from C₁-C₆ alkyl (optionally substituted with 1-3 groups independently selected from hydroxyl and oxo),
   C₆-C₁₀ aryl optionally substituted with 1-4 groups independently selected from:
      halogen,
      cyano,
      C₁-C₇ alkyl optionally substituted with 1-3 groups independently selected from oxo and N(R^{N})₂,
      C₃-C₁₀ cycloalkyl,
      3- to 10-membered heterocyclyl optionally substituted with 1-3 groups independently selected from C₁-C₆ alkyl, and
      5- to 10-membered heteroaryl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl.
125. The compound, tautomer, deuterated derivative, or pharmaceutically acceptable salt according to any one of embodiments 115 to 124, wherein each C(R^{L2})₂ is CH₂ or C═O.
126. The compound, tautomer, deuterated derivative, or pharmaceutically acceptable salt according to any one of embodiments 115 to 125, wherein each R^{N} is independently selected from:
   hydrogen,
   C₁-C₈ alkyl optionally substituted with 1-3 groups independently selected from:
      oxo,
      hydroxyl,
      C₁-C₆ alkoxy,
      —(O)₀₋₁—(C₃-C₁₀ cycloalkyl),
      C₆-C₁₀ aryl,
      3- to 14-membered heterocyclyl,
      5- to 14-membered heteroaryl optionally substituted with 1-4 groups independently selected from C₁-C₆ alkyl,
   C₃-C₁₀ cycloalkyl optionally substituted with 1-3 groups independently selected from C₁-C₆ alkyl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from C₁-C₆ alkyl.
127. The compound, tautomer, deuterated derivative, or pharmaceutically acceptable salt according to any one of embodiments 115 to 126, wherein when R^{F} is present, two R^{F}taken together with the atoms to which they are bonded form a group selected from 3- to 11-membered heterocyclyl optionally substituted with 1-3 groups independently selected from:
   C₁-C₉ alkyl, and
   5- to 10-membered heteroaryl optionally substituted with 1-3 groups independently selected from C₁-C₆ alkoxy.
128. A compound of Formula VI:

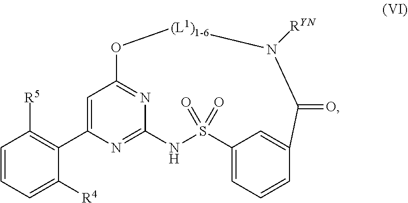

a tautomer thereof, a deuterated derivative of the compound or tautomer, or a pharmaceutically acceptable salt of any of the foregoing, wherein L¹, R⁴, R⁵, and R^{YN}are defined as according to embodiment 1a.
129. The compound, tautomer, deuterated derivative, or pharmaceutically acceptable salt according to embodiment 128, wherein each L¹ is C(R^{L1})₂.
130. The compound, tautomer, deuterated derivative, or pharmaceutically acceptable salt according to embodiment 128 or 129, wherein R⁴ is selected from hydrogen and methyl.
131. The compound, tautomer, deuterated derivative, or pharmaceutically acceptable salt according to any one of embodiments 128 to 130, wherein R⁴ is methyl.
132. The compound, tautomer, deuterated derivative, or pharmaceutically acceptable salt according to any one of embodiments 128 to 131, wherein each R⁵ is independently selected from:
   hydrogen,
   hydroxyl,
   C₁-C₆ alkyl optionally substituted with 1-3 groups independently selected from C₃-C₁₀ cycloalkyl,
   C₁-C₆ alkoxy optionally substituted with 1-3 groups independently selected from C₆-C₁₀ aryl,
133. The compound, tautomer, deuterated derivative, or pharmaceutically acceptable salt according to any one of embodiments 128 to 132, wherein R^{YN} is selected from:
   C₁-C₉ alkyl optionally substituted with 1-3 groups independently selected from:
      hydroxyl,
      oxo,
      cyano,
      C₁-C₆ alkoxy optionally substituted with 1-3 groups independently selected from halogen and C₁-C₆ alkoxy,
      N(R^{N})₂,
      SO₂Me,
      C₃-C₁₀ cycloalkyl optionally substituted with 1-3 groups independently selected from:
         C₁-C₆ alkyl optionally substituted with 1-3 groups independently selected from oxo and N(R^{N})₂,
         C₁-C₆ fluoroalkyl,
         C₁-C₆ alkoxy, and
         COOH,
      C₆-C₁₀ aryl optionally substituted with 1-3 groups independently selected from:
         halogen,
         hydroxyl,
         cyano,
         SiMe₃,
         SO₂Me,
         SF₅,
         N(R^{N})₂,
         P(O)Me₂,
         —(O)₀₋₁—(C₃-C₁₀ cycloalkyl) optionally substituted with 1-3 groups independently selected from C₁-C₆ fluoroalkyl,
         C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
         C₁-C₆ alkoxy optionally substituted with 1-3 groups independently selected from hydroxyl, oxo, N(R^{N})₂, and C₆-C₁₀ aryl,
         C₁-C₆ fluoroalkyl,
         3- to 10-membered heterocyclyl optionally substituted with 1-3 groups independently selected from C₁-C₆ alkyl, and
         —(O)₀₋₁—(C₆-C₁₀ aryl),
      3- to 10-membered heterocyclyl optionally substituted with 1-4 groups independently selected from oxo and C₁-C₆ alkoxy, and
      5- to 10-membered heteroaryl optionally substituted with 1-3 groups independently selected from:
         hydroxyl,
         cyano,
         oxo,
         halogen,
         B(OH)₂,
         N(R^{N})₂,
         C₁-C₆ alkyl optionally substituted with 1-3 groups independently selected from hydroxyl, oxo, C₁-C₆ alkoxy, and N(R^{N})₂,
         C₁-C₆ alkoxy optionally substituted with 1-3 groups independently selected from hydroxyl, C₁-C₆ alkoxy, N(R^{N})₂, and C₃-C₁₀ cycloalkyl,
         C₁-C₆ fluoroalkyl,
         —(O)₀₋₁—(C₃-C₁₀ cycloalkyl) optionally substituted with 1-3 groups independently selected from C₁-C₆ alkyl,
         —(O)₀₋₁—(C₆-C₁₀ aryl),
         —(O)₀₋₁—(3- to 10-membered heterocyclyl) optionally substituted with 1-4 groups independently selected from C₁-C₆ alkyl (optionally substituted with 1-3 groups independently selected from hydroxyl), and
         5- to 10-membered heteroaryl optionally substituted with 1-4 groups independently selected from C₁-C₆ alkyl.
134. The compound, tautomer, deuterated derivative, or pharmaceutically acceptable salt according to any one of embodiments 128 to 133, wherein each R^{L1} is independently selected from:
   hydrogen,
   C₁-C₉ alkyl optionally substituted with 1-3 groups independently selected from:
      halogen,
      hydroxyl,
      oxo,
      N(R^{N})₂,
      C₁-C₆ alkoxy optionally substituted with 1-3 groups independently selected from C₆-C₁₀ aryl,
      C₃-C₁₀ cycloalkyl optionally substituted with 1-3 groups independently selected from C₁-C₆ fluoroalkyl, and
      3- to 10-membered heterocyclyl optionally substituted with 1-3 groups independently selected from C₁-C₆ alkyl (optionally substituted with 1-3 groups independently selected from hydroxyl and oxo),
   C₆-C₁₀ aryl optionally substituted with 1-4 groups independently selected from:
      halogen,
      cyano,
      C₁-C₇ alkyl optionally substituted with 1-3 groups independently selected from oxo and N(R^{N})₂,
      C₃-C₁₀ cycloalkyl,
      3- to 10-membered heterocyclyl optionally substituted with 1-3 groups independently selected from C₁-C₆ alkyl, and
      5- to 10-membered heteroaryl,
   5- to 10-membered heteroaryl optionally substituted with 1-3 groups independently selected from:
      C₁-C₆ alkyl optionally substituted with 1-3 groups independently selected from:
         C₃-C₁₀ cycloalkyl optionally substituted with 1-3 groups independently selected from C₁-C₆ fluoroalkyl, and
      C₆-C₁₀ aryl optionally substituted with 1-3 groups independently selected from C₁-C₆ alkyl.
135. The compound, tautomer, deuterated derivative, or pharmaceutically acceptable salt according to any one of embodiments 128 to 134, wherein each R^{N} is independently selected from:
   hydrogen,
   C₁-C₈ alkyl optionally substituted with 1-3 groups independently selected from:
      oxo,
      hydroxyl,
      C₁-C₆ alkoxy,
      —(O)₀₋₁—(C₃-C₁₀ cycloalkyl),
      C₆-C₁₀ aryl,
      3- to 14-membered heterocyclyl,
      5- to 14-membered heteroaryl optionally substituted with 1-4 groups independently selected from C₁-C₆ alkyl,
   C₃-C₁₀ cycloalkyl optionally substituted with 1-3 groups independently selected from C₁-C₆ alkyl, and
   3- to 10-membered heterocyclyl;
or two R^{N} on the same nitrogen atom are taken together with the nitrogen to which they are bonded to form a 3- to 10-membered heterocyclyl optionally substituted with 1-3 groups selected from C₁-C₆ alkyl.
136. The compound, tautomer, deuterated derivative, or pharmaceutically acceptable salt according to any one of embodiments 128 to 135, wherein when R^{F} is present, two R^{F}taken together with the atoms to which they are bonded form a group selected from 3- to 11-membered heterocyclyl optionally substituted with 1-3 groups independently selected from:
   C₁-C₉ alkyl, and
   5- to 10-membered heteroaryl optionally substituted with 1-3 groups independently selected from C₁-C₆ alkoxy.
137. The compound, tautomer, deuterated derivative, or pharmaceutically acceptable salt according to any one of embodiments 1 to 136, selected from compounds of any one of Formulae I, Ia, Ib, IIa, IIb, III, IV, V, and VI, tautomers thereof, deuterated derivatives of those compounds and tautomers, and pharmaceutically acceptable salts of any of the foregoing.
138. The compound, tautomer, deuterated derivative, or pharmaceutically acceptable salt according any one of embodiments 1 to 137, selected from Compounds 1-1193 (Tables 3 and 6-13), Compounds 1194-1294 (Table 5), Compounds 1295-1972 (Tables 14-16) tautomers thereof, deuterated derivatives of those compounds and tautomers, and pharmaceutically acceptable salts of any of the foregoing.
139. A pharmaceutical composition comprising the compound, tautomer, deuterated derivative, or pharmaceutically acceptable salt according to any one of embodiments 1 to 138, and a pharmaceutically acceptable carrier.
140. The pharmaceutical composition of embodiment 139, further comprising one or more additional therapeutic agents.
141. The pharmaceutical composition of embodiment 140, wherein the one or more additional therapeutic agents is selected from mucolytic agents, bronchodilators, antibiotics, anti-infective agents, and anti-inflammatory agents.
142. The pharmaceutical composition of embodiment 141, wherein the one or more additional therapeutic agent is an antibiotic selected from tobramycin, including tobramycin inhaled powder (TIP), azithromycin, aztreonam, including the aerosolized form of aztreonam, amikacin, including liposomal formulations thereof, ciprofloxacin, including formulations thereof suitable for administration by inhalation, levoflaxacin, including aerosolized formulations thereof, and combinations of two antibiotics, e.g., fosfomycin and tobramycin.
143. The pharmaceutical composition of embodiment 140, wherein the one or more additional therapeutic agent is one or more CFTR modulating agents.
144. The pharmaceutical composition of embodiment 143, wherein the one or more CFTR modulating agents are selected from CFTR potentiators.
145. The pharmaceutical composition of embodiment 143, wherein the one or more CFTR modulating agents are selected from CFTR correctors.
146. The pharmaceutical composition of embodiment 143, wherein the one or more CFTR modulating agents comprises at least one CFTR potentiator and at least one CFTR corrector.
147. The pharmaceutical composition of any one of embodiment 143-146, wherein the one or more CFTR modulating agents are selected from (a) tezacaftor, lumacaftor, and deuterated derivatives and pharmaceutically acceptable salts thereof; and (b) ivacaftor, deutivacaftor, and deuterated derivatives and pharmaceutically acceptable salts of any of the foregoing.
148. The pharmaceutical composition of any one of embodiments 143-146, wherein the one or more CFTR modulating agents are selected from (a) tezacaftor, lumacaftor, and deuterated derivatives and pharmaceutically acceptable salts thereof; or (b) (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol and deuterated derivatives and pharmaceutically acceptable salts of any of the foregoing.
149. The pharmaceutical composition of any one of embodiment 143-147, wherein the composition comprises tezacaftor and ivacaftor.
150. The pharmaceutical composition of any one of embodiment 143-147, wherein the composition comprises tezacaftor and deutivacaftor.
151. The pharmaceutical composition of any one of embodiment 143-147, wherein the composition comprises tezacaftor and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.
152. The pharmaceutical composition of any one of embodiment 143-147, wherein the composition comprises lumacaftor and ivacaftor.
153. The pharmaceutical composition of any one of embodiment 143-147, wherein the composition comprises lumacaftor and deutivacaftor.
154. The pharmaceutical composition of any one of embodiment 143-147, wherein the composition comprises lumacaftor and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.
155. A method of treating cystic fibrosis comprising administering to a patient in need thereof the compound, tautomer, deuterated derivative, or pharmaceutically acceptable salt according to any one of embodiments 1 to 138, or a pharmaceutical composition according to any one of embodiments 139 to 154.
156. The method of embodiment 155, further comprising administering to the patient one or more additional therapeutic agents prior to, concurrent with, or subsequent to the compound, tautomer, deuterated derivative, or pharmaceutically acceptable salt according to any one of embodiments 1 to 138, or the pharmaceutical composition according to any one of embodiments 139.
157. The method of embodiment 156, wherein the one or more additional therapeutic agents is(are) selected from CFTR modulating agents.
158. The method of embodiment 157, wherein the one or more CFTR modulating agents are selected from CFTR potentiators.
159. The method of embodiment 157, wherein the one or more CFTR modulating agents are selected from CFTR correctors.
160. The method of embodiment 157, wherein the one or more CFTR modulating agents comprising both a CFTR potentiator and an additional CFTR corrector.
161. The method of embodiment 158 and 160, wherein the CFTR potentiator is selected from ivacaftor, deutivacaftor, (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol, and deuterated derivatives and pharmaceutically acceptable salts of any of the foregoing.
162. The method of embodiment 159 or embodiment 160, wherein the CFTR corrector is selected from tezacaftor, lumacaftor, and deuterated derivatives and pharmaceutically acceptable salts thereof.
163. The method of embodiment 156, wherein the one or more additional therapeutic agent(s) is a compound selected from tezacaftor, ivacaftor, deutivacaftor, lumacaftor, and pharmaceutically acceptable salts thereof.
164. The method of embodiment 163, wherein the one or more additional therapeutic agents are tezacaftor and ivacaftor.
165. The method of embodiment 163, wherein the one or more additional therapeutic agents are tezacaftor and deutivacaftor.
166. The method of embodiment 163, wherein the one or more additional therapeutic agents are tezacaftor and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.
167. The method of embodiment 163, wherein the one or more additional therapeutic agents are lumacaftor and ivacaftor.
168. The method of embodiment 163, wherein the one or more additional therapeutic agents are lumacaftor and deutivacaftor.
169. The method of embodiment 163, wherein the one or more additional therapeutic agents are lumacaftor and (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol.
170. The compound, tautomer, deuterated derivative, or pharmaceutically acceptable salt according to any one of embodiments 1 to 138, or the pharmaceutical composition according to any one of embodiments 139 to 154, for use in the treatment of cystic fibrosis.
171. The compound, tautomer, deuterated derivative, or pharmaceutically acceptable salt according to any one of embodiments 1 to 138, or the pharmaceutical composition according to any one of embodiments 139 to 154, for use in the manufacture of a medicament for the treatment of cystic fibrosis.
172. A compound selected from Compounds 1-1972, tautomers thereof, deuterated derivatives of those compounds and tautomers, and pharmaceutically acceptable salts of any of the foregoing.
173. A deuterated derivative of a compound selected from Compounds 1-1972.
174. A pharmaceutically acceptable salt of a compound selected from Compounds 1-1972.
175. A compound selected from Compounds 1-1972.
176. A pharmaceutical composition comprising a compound selected from Compounds 1-1972, tautomers thereof, deuterated derivatives of those compounds and tautomers, and pharmaceutically acceptable salts of any of the foregoing and a pharmaceutically acceptable carrier.
177. A pharmaceutical composition comprising a deuterated derivative of a compound selected from Compounds 1-1972 and a pharmaceutically acceptable carrier.
178. A pharmaceutical composition comprising a pharmaceutically acceptable salt of a compound selected from Compounds 1-1972 and a pharmaceutically acceptable carrier.
179. A pharmaceutical composition comprising a compound selected from Compounds 1-1972 and a pharmaceutically acceptable carrier.
180. A pharmaceutical composition comprising (a) a compound selected from Compounds 1-1972, tautomers thereof, deuterated derivatives of those compounds and tautomers, and pharmaceutically acceptable salts of any of the foregoing; (b) a CFTR potentiator; and (c) a pharmaceutically acceptable carrier.
181. A pharmaceutical composition composition comprising (a) a deuterated derivative of a compound selected from Compounds 1-1972; (b) a CFTR potentiator; and (c) a pharmaceutically acceptable carrier.
182. A pharmaceutical comprising (a) a pharmaceutically acceptable salt of a compound selected from Compounds 1-1972; (b) a CFTR potentiator; and (c) a pharmaceutically acceptable carrier.
183. A pharmaceutical composition comprising (a) a compound selected from Compounds 1-1972; (b) a CFTR potentiator; and (c) a pharmaceutically acceptable carrier.
184. A pharmaceutical composition comprising (a) a compound selected from Compounds 1-1972, tautomers thereof, deuterated derivatives of those compounds and tautomers, and pharmaceutically acceptable salts of any of the foregoing; (b) an additional CFTR corrector; and (c) a pharmaceutically acceptable carrier.
185. A pharmaceutical composition comprising (a) a deuterated derivative of a compound selected from Compounds 1-1972; (b) an additional CFTR corrector; and (c) a pharmaceutically acceptable carrier.
186. A pharmaceutical composition comprising (a) a pharmaceutically acceptable salt of a compound selected from Compounds 1-1972; (b) an additional CFTR corrector; and (c) a pharmaceutically acceptable carrier.
187. A pharmaceutical composition comprising (a) a compound selected from Compounds 1-1972; (b) an additional CFTR corrector; and (c) a pharmaceutically acceptable carrier.
188. A pharmaceutical composition comprising (a) a compound selected from Compounds 1-1972, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier.
189. A pharmaceutical composition comprising (a) a deuterated derivative of a compound selected from Compounds 1-1972; (b) an additional CFTR corrector; (c) a CFTR potentiator; and (d) a pharmaceutically acceptable carrier.
190. A pharmaceutical composition comprising (a) a pharmaceutically acceptable salt of a compound selected from Compounds 1-1972; (b) an additional CFTR corrector; (c) a CFTR potentiator; and (d) a pharmaceutically acceptable carrier.
191. A pharmaceutical composition comprising (a) a compound selected from Compounds 1-1972; (b) an additional CFTR corrector; (c) a CFTR potentiator; and (d) a pharmaceutically acceptable carrier.
192. A compound selected from Compounds 1-1972, tautomers thereof, deuterated derivatives of those compounds and tautomers, and pharmaceutically acceptable salts of any of the foregoing for use in a method of treating cystic fibrosis.
193. A deuterated derivative of a compound selected from Compounds 1-1972 for use in a method of treating cystic fibrosis.
194. A pharmaceutically acceptable salt of a compound selected from Compounds 1-1972 for use in a method of treating cystic fibrosis.
195. A compound selected from Compounds 1-1972 for use in a method of treating cystic fibrosis.
196. A pharmaceutical composition comprising a compound selected from Compounds 1-1972, tautomers thereof, deuterated derivatives of those compounds and tautomers, and pharmaceutically acceptable salts of any of the foregoing and a pharmaceutically acceptable carrier for use in a method of treating cystic fibrosis.
197. A pharmaceutical composition comprising a deuterated derivative of a compound selected from Compounds 1-1972 and a pharmaceutically acceptable carrier for use in a method of treating cystic fibrosis.
198. A pharmaceutical composition comprising a pharmaceutically acceptable salt of a compound selected from Compounds 1-1972 and a pharmaceutically acceptable carrier for use in a method of treating cystic fibrosis.
199. A pharmaceutical composition comprising a compound selected from Compounds 1-1972 and a pharmaceutically acceptable carrier for use in a method of treating cystic fibrosis.
200. A pharmaceutical composition comprising (a) a compound selected from Compounds 1-1972, tautomers thereof, deuterated derivatives of those compounds and tautomers, and pharmaceutically acceptable salts of any of the foregoing; (b) a CFTR potentiator; and (c) a pharmaceutically acceptable carrier for use in a method of treating cystic fibrosis.
201. A pharmaceutical comprising (a) a deuterated derivative of a compound selected from Compounds 1-1972; (b) a CFTR potentiator; and (c) a pharmaceutically acceptable carrier for use in a method of treating cystic fibrosis.
202. A pharmaceutical composition comprising (a) a pharmaceutically acceptable salt of a compound selected from Compounds 1-1972; (b) a CFTR potentiator; and (c) a pharmaceutically acceptable carrier for use in a method of treating cystic fibrosis.
203. A pharmaceutical composition comprising (a) a compound selected from Compounds 1-1972; (b) a CFTR potentiator; and (c) a pharmaceutically acceptable carrier.
204. A pharmaceutical composition comprising (a) a compound selected from Compounds 1-1972, tautomers thereof, deuterated derivatives of those compounds and tautomers, and pharmaceutically acceptable salts of any of the foregoing; (b) an additional CFTR corrector; and (c) a pharmaceutically acceptable carrier for use in a method of treating cystic fibrosis.
205. A pharmaceutical composition comprising (a) a deuterated derivative of a compound selected from Compounds 1-1972; (b) an additional CFTR corrector; and (c) a pharmaceutically acceptable carrier for use in a method of treating cystic fibrosis.
206. A pharmaceutical composition comprising (a) a pharmaceutically acceptable salt of a compound selected from Compounds 1-1972; (b) an additional CFTR corrector; and (c) a pharmaceutically acceptable carrier for use in a method of treating cystic fibrosis.
207. A pharmaceutical composition comprising (a) a compound selected from Compounds 1-1972; (b) an additional CFTR corrector; and (c) a pharmaceutically acceptable carrier for use in a method of treating cystic fibrosis.
208. A pharmaceutical composition comprising (a) a compound selected from Compounds 1-1972, tautomers thereof, deuterated derivatives of those compounds and tautomers, and pharmaceutically acceptable salts of any of the foregoing; (b) an additional CFTR corrector; (c) a CRTR potentiator; and (d) a pharmaceutically acceptable carrier for use in a method of treating cystic fibrosis.
209. A pharmaceutical composition comprising (a) a deuterated derivative of a compound selected from Compounds 1-1972; (b) an additional CFTR corrector; (c) a CFTR potentiator; and (d) a pharmaceutically acceptable carrier for use in a method of treating cystic fibrosis.
210. A pharmaceutical composition comprising (a) a pharmaceutically acceptable salt of a compound selected from Compounds 1-1972; (b) an additional CFTR corrector; (c) a CFTR potentiator; and (d) a pharmaceutically acceptable carrier for use in a method of treating cystic fibrosis.
211. A pharmaceutical composition comprising (a) a compound selected from Compounds 1-1972; (b) an additional CFTR corrector; (c) a CFTR potentiator; and (d) a pharmaceutically acceptable carrier for use in a method of treating cystic fibrosis.

### EXAMPLES

### I. Abbreviation List

ACN: Acetonitrile
Boc anhydride ((Boc)₂O): Di-tert-butyl decarbonate
CDCl₃: Chloroform-d CDI: Carbonyl diimidazole
CDI: Carbonyl diimidazole
CDMT: 2-Chloro-4,6-dimethoxy-1,3,5-triazine
CH₂Cl₂: Dichloromethane
CH₃CN: Acetonitrile
COMU: (1-Cyano-2-ethoxy-2-oxoethylidenaminooxy)dimethylamino-morpholino-carbenium hexafluorophosphate
Cmpd: Compound
DABCO: 1,4-Diazabicyclo[2.2.2]octane
DBU: 1,8-Diazabicyclo(5.4.0)undec-7-ene
DCE: 1,2-Dichloroethane
DCM: Dichloromethane
DI: Deionized
DIAD: Diisopropyl azodicarboxylate
DIEA: (DIPEA, DiPEA): N,N-diisopropylethylamine
DMA: N,N-Dimethylacetamide
DMAP: 4-Dimethylaminopyridine
DMF: N,N-Dimethylformamide
DMSO: Dimethyl sulfoxide
DMP: Dess-Martin periodinane
EA: Ethyl acetate
EDC: 1-Ethyl-3-(3-dimethylaminopropyl)carbodiimide
ELSD: Evaporative light scattering detector
diethylether: Diethyl ether
ESI-MS: Electrospray ionization mass spectrometry
EtOAc: Ethyl acetate
EtOH: Ethanol
GC: Gas chromatography
Grubbs 1^{st} Generation catalyst: Dichloro(benzylidene)bis(tricyclohexylphosphine)ruthenium(II)
Grubbs 2^{nd} Generation catalyst: [1,3-Bis(2,4,6-trimethylphenyl)imidazolidin-2-ylidene]-dichloro-[(2-isopropoxyphenyl)methylene]ruthenium
HATU: 1-[Bis(dimethylamino)methylene]-1H-1,2,3-triazolo[4,5-b]pyridinium 3-oxid hexafluorophosphate
HPLC: High-performance liquid chromatography
Hoveyda-Grubbs 2^{nd} Generation catalyst: (1,3-Bis-(2,4,6-trimethylphenyl)-2-imidazolidinylidene)dichloro(o-isopropoxyphenylmethylene)ruthenium, Dichloro[1,3-bis(2,4,6-trimethylphenyl)-2-imidazolidinylidene](2-isopropoxyphenylmethylene)ruthenium(II)
IPA: Isopropanol
KHSO₄: Potassium bisulfate
LC: Liquid chromatography
LCMS: Liquid chromatography mass spectrometry
LCMS Met: LCMS method
LCMS Rt: LCMS retention time
LDA: Lithium diisopropylamide
LiOH: Lithium hydroxide
MeCN: Acetonitrile
MeOH: Methanol
MTBE: Methyl tert-butyl ether
MeTHF or 2-MeTHF: 2-Methyltetrahydrofuran
MgSO₄: Magnesium sulfate
NaHCO₃: Sodium bicarbonate
NaOH: Sodium hydroxide
NMP: N-Methyl-2-pyrrolidone
NMM: N-Methylmorpholine
Pd₂(dba)₃: Tris(dibenzylideneacetone)dipalladium(O)
Pd/C: Palladium on carbon
Pd(dppf)Cl₂: [1,1′-Bis(diphenylphosphino)ferrocene]dichloropalladium(II)
Pd(OAc)₂: Palladium(II) acetate
PTFE: Polytetrafluoroethylene
rt, RT: Room temperature
RuPhos: 2-Dicyclohexylphosphino-2′,6′-diisopropoxybiphenyl
SFC: Supercritical fluid chromatography
TBAI: Tetrabutylammonium iodide
TEA: Triethylamine
TFA: Trifluoroacetic acid
THF: Tetrahydrofuran
TLC: Thin layer chromatography
TMS: Trimethylsilyl
TMSCl: Trimethylsilyl chloride
T3P: Propanephosphonic acid anhydride
UPLC: Ultra Performance Liquid Chromatography
XANTPHOS: 4,5-Bis(diphenylphosphino)-9,9-dimethylxanthene
XPhos: 2-Dicyclohexylphosphino-2′,4′,6′-triisopropylbiphenyl

### II. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. 19F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before Fourier transform. ¹⁹F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Final purity of compounds was determined by reversed phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range. Optical purity of methyl (2S)-2,4-dimethyl-4-nitro-pentanoate was determined using chiral gas chromatography (GC) analysis on an Agilent 7890A/MSD 5975C instrument, using a Restek Rt-βDEXcst (30 m×0.25 mm×0.25 μm_df) column, with a 2.0 mL/min flow rate (H₂ carrier gas), at an injection temperature of 220° C. and an oven temperature of 120° C., 15 minutes.

### III. General UPLC/HPLC/GC Analytical Methods

LC method A: Analytical reverse phase UPLC using an Acquity UPLC BEH Cis column (50×2.1 mm, 1.7 μm particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 L, and column temperature=60° C.

LC method C: Kinetex C₁₈ 4.6×50 mm 2.6 μm. Temp: 45° C., Flow: 2.0 mL/min, Run Time: 3 min. Mobile phase: Initial 95% water (0.1% formic acid) and 5% acetonitrile (0.1% formic acid) linear gradient to 95% acetonitrile (0.1% formic acid) for 2.0 min then hold at 95% acetonitrile (0.1% formic acid) for 1.0 min.

LC method D: Acquity UPLC BEH C₁₈ column (30×2.1 mm, 1.7 μm particle) made by Waters (pn: 186002349), and a dual gradient run from 1-99% mobile phase B over 1.0 minute. Mobile phase A=H₂O (0.05% CF₃CO₂H). Mobile phase B=CH₃CN (0.035% CF₃CO₂H). Flow rate=1.5 mL/min, injection volume=1.5 μL, and column temperature=60° C.

LC method G: Symmetry, 4.6×75 mm 3.5 μm. Temp: 45° C., Flow: 2.0 mL/min, Run Time: 8 min. Mobile Phase: Initial 95% H₂O (0.1% Formic Acid) and 5% CH₃CN (0.1% FA) linear gradient to 95% CH₃CN (0.1% formic acid) for 6.0 min then hold at 95% CH₃CN (0.1% formic acid) for 2.0 min.

LC method H: Kinetex C₁₈ 4.6×50 mm 2.6 um. Temp: 45° C., Flow: 2.0 mL/min, Run Time: 6 min. Mobile Phase: Initial 95% H₂O (0.1% Formic Acid) and 5% CH₃CN (0.1% FA) linear gradient to 95% CH₃CN (0.1% FA) for 4.0 min then hold at 95% CH₃CN (0.1% FA) for 2.0 min.

LC method I: Acquity UPLC BEH C₁₈ column (50×2.1 mm, 1.7 μm particle) made by Waters (pn:186002350), and a dual gradient run from 1-99% mobile phase B over 5.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method J: Reverse phase UPLC using an Acquity UPLC BEH C₁₈ column (50×2.1 mm, 1.7 μm particle) made by Waters (pn: 186002350), and a dual gradient run from 1-99% mobile phase B over 2.9 minutes. Mobile phase A=H₂O (0.05% NH₄HCO₂). Mobile phase B=CH₃CN. Flow rate=1.2 mL/min, injection volume=1.5 μL, and column temperature=60° C.

LC method K: Kinetex Polar C₁₈ 3.0×50 mm 2.6 μm, 3 min, 5-95% ACN in H₂O (0.1% Formic Acid) 1.2 ml/min.

LC method Q: Reversed phase UPLC using an Acquity UPLC BEH C₁₈ column (50×2.1 mm, 1.7 μm particle) made by Waters (pn: 186002350), and a dual gradient run from 30-99% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method S: Merckmillipore Chromolith SpeedROD C₁₈ column (50×4.6 mm) and a dual gradient run from 5-100% mobile phase B over 12 minutes. Mobile phase A=water (0.1% CF₃CO₂H). Mobile phase B=acetonitrile (0.1% CF₃CO₂H).

LC method T: Merckmillipore Chromolith SpeedROD C₁₈ column (50×4.6 mm) and a dual gradient run from 5-100% mobile phase B over 6 minutes. Mobile phase A=water (0.1% CF₃CO₂H). Mobile phase B=acetonitrile (0.1% CF₃CO₂H).

LC method U: Kinetex Polar C₁₈ 3.0×50 mm 2.6 μm, 6 min, 5-95% ACN in H₂O (0.1% Formic Acid) 1.2 mL/min.

LC method V: Acquity UPLC BEH C₁₈ column (50×2.1 mm, 1.7 μm particle) made by Waters (pn: 186002350), and a dual gradient run from 1-30% mobile phase B over 2.9 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

LC method W: water Cortex 2.7μ C₁₈(3.0 mm×50 mm), Temp: 55° C.; Flow: 1.2 mL/min; mobile phase: 100% water with 0.1% trifluoroacetic(TFA) acid then 100% acetonitrile with 0.1% TFA acid, grad:5% to 100% B over 4 min, with stay at 100% B for 0.5 min, equilibration to 5% B over 1.5 min.

LC method X: UPLC Luna C₁₈(2) 50×3 mm 3 m. run: 2.5 min. Mobile phase: Initial 95% H₂O 0.1% FA/5% MeCN 0.1% FA, linear grad to 95% MeCN 0.1% FA over 1.3 min, hold 1.2 min 95% CH₃CN 0.1% FA.T: 45C, Flow: 1.5 mL/min

LC method Y: UPLC SunFire C₁₈ 75×4.6 mm 3.5 m, run: 6 min. Mobile phase conditions: Initial 95% H₂O+0.1% FA/5% CH₃CN+0.1% FA, linear gradient to 95% CH₃CN for 4 min, hold for 2 min at 95% CH₃CN. T:45° C., Flow:1.5 mL/min

LC method 1A: Reversed phase UPC2 using a Viridis BEH 2-Ethylpyridine column (150×2.1 mm, 3.5 μm particle) made by Waters (pn: 186006655), and a dual gradient run from 5-80% mobile phase B over 4.5 minutes. Mobile phase A=C02. Mobile phase B=MeOH (20 mM NH₃). Variable flow rate=1.30-0.40 mL/min to maintain constant pressure, injection volume=2.0 μL, and column temperature=55° C.

GC method 1B: Column SPB-1.30 m×0.32 mm×0.25 um. Control mode: head pressure 100 kPa. Split ratio mode: 10.0. Carrier gas: hydrogen. Injector temperature: 150° C. Detector temperature: 250° C. Oven temperature: isotherm at 40° C. for 1 min, then linear heating at 10° C./min until 100° C., then 20° C./min until 220° C. then isotherm 220° C. for 4 min. Run time 17.0 minutes. Non chiral method.

LC method 1C: Luna C₁₈(2) 3.0×50 mm 3 m, run: 5 min. Mobile phase conditions: Initial 95% H₂O 0.05% TFA 5% CH₃CN, linear gradient to 5% H₂O 0.05% TFA 95% CH₃CN for 3.5 min, hold at 95% CH₃CN for 1.5 min, T: 45° C., Flow: 1.2 mL/min.

LC method 1D: XBridge C₁₈ 4.6×75 mm, 5 m, Initial Gradient at 95% NH₄HCO₃/5% MeCN 6 min run with 1 min equilibration gradient 0 to 3 min at 95% MeCN and hold for 3 minutes. Flow 1.5 mL/min.

LC method 1E: reversed phase UPLC using an Acquity UPLC BEH C₁₈ column (30×2.1 mm, 1.7 μm particle) made by Waters (pn: 186002349), and a dual gradient run from 1-99% mobile phase B over 2.9 minutes. Mobile phase A=H₂O (5 mM NH₄OH). Mobile phase B=CH₃CN. Flow rate=1.1 mL/min, injection volume=1.5 μL, and column temperature=60° C.

### IV. Synthesis of Common Intermediates

### Example A: Preparation of 3-[[4-Chloro-6-(2,6-dimethylphenyl)pyrimidin-2-yl]sulfamoyl]benzoic acid

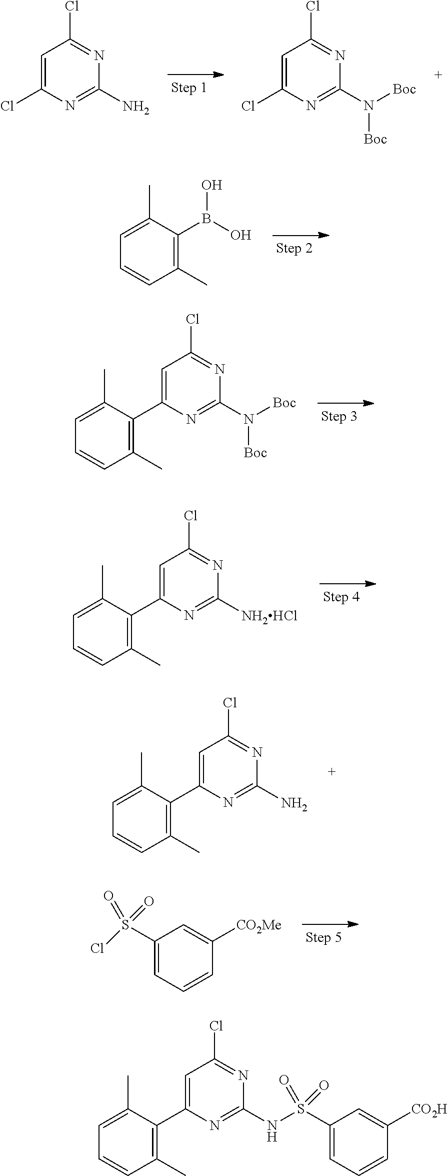

### Step 1: tert-Butyl N-tert-butoxycarbonyl-N-(4,6-dichloropyrimidin-2-yl)carbamate

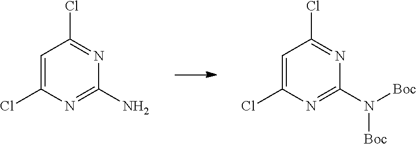 [SMILES: Nc1nc(Cl)cc(Cl)n1 ; CC(C)(C)OC(=O)N(C(=O)OC(C)(C)C)c1nc(Cl)cc(Cl)n1]

To a solution of 4,6-dichloropyrimidin-2-amine (300 g, 1.829 mol) in DCM (2.1 L) was added (BOC)₂O (838 g, 3.840 mol) followed by DMAP (5.6 g, 45.84 mmol). The mixture was stirred at ambient temperature for 6 h. Additional DMAP (5.6 g, 45.84 mmol) was added and the reaction was continued to stir at ambient temperature for 24 h. The mixture was diluted with water (2.1 L) and the organic phase separated. The organic phase was washed with water (2.1 L), 2.1 L of brine, dried over magnesium sulfate, filtered over Celite and concentrated in vacuo affording a light orange oil which had a silt in the slurry. The mixture was diluted with −500 mL of heptane and filtered using an M filter. The precipitate (SM) was washed with 250 mL of heptane. The filtrate was concentrated in vacuo affording a thick orange oil which was seeded with solid from a previous experiment and crystallized on standing, affording a light orange hard solid. tert-butyl N-tert-butoxycarbonyl-N-(4,6-dichloropyrimidin-2-yl)carbamate (645 g, 97%). ¹H NMR (400 MHz, DMSO-d₆) δ 8.07 (s, 1H), 1.44 (s, 18H). ESI-MS m/z calc. 363.07526, found 364.1 (M+1)⁺; Retention time: 2.12 minutes (LC method A).

### Step 2: tert-Butyl N-tert-butoxycarbonyl-N-[4-chloro-6-(2,6-dimethylphenyl)pyrimidin-2-yl]carbamate

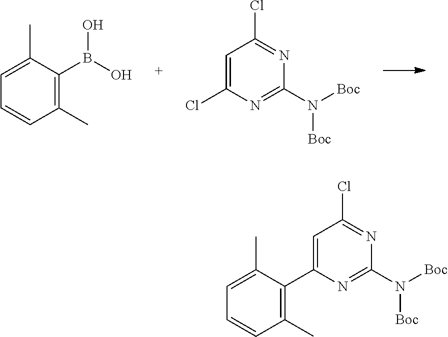 [SMILES: Cc1cccc(C)c1B(O)O ; CC(C)(C)OC(=O)N(C(=O)OC(C)(C)C)c1nc(Cl)cc(Cl)n1 ; Cc1cccc(C)c1-c1cc(Cl)nc(N(C(=O)OC(C)(C)C)C(=O)OC(C)(C)C)n1]

All solvents were degassed prior to use. To a slurry of tert-butyl N-tert-butoxycarbonyl-N-(4,6-dichloropyrimidin-2-yl)carbamate (88 g, 241.6 mmol), (2,6-dimethylphenyl)boronic acid (approximately 36.24 g, 241.6 mmol) and Cs₂CO₃ (approximately 196.8 g, 604.0 mmol) in DME (704 mL) and water (176 mL) were added. Pd(dppf)Cl₂ (approximately 8.839 g, 12.08 mmol) was added and the mixture was vigorously stirred under nitrogen at 80° C. (reflux) for 1 h (no SM remained). The reaction was cooled to ambient temperature and diluted with water (704 mL). The aqueous phase was separated and extracted with EtOAc (704 mL). The organic phase was washed with 700 mL of brine, dried over magnesium sulfate, filtered and concentrated in vacuo. The crude product was chromatographed on a 1500 g silica gel column eluting with 0-30% EtOAc/hexanes. The product fractions (eluted at 15% EtOAc) were combined and concentrated in vacuo affording the product as a clear oil which crystallized on standing. tert-butyl N-tert-butoxycarbonyl-N-[4-chloro-6-(2,6-dimethylphenyl)pyrimidin-2-yl]carbamate (81.3 g, 78%). ¹H NMR (400 MHz, DMSO-d₆) δ 7.88 (s, 1H), 7.30 (dd, J 8.2, 7.0 Hz, 1H), 7.21-7.16 (m, 2H), 2.03 (s, 6H), 1.38 (s, 18H). ESI-MS m/z calc. 433.17682, found 434.1 (M+1)⁺; Retention time: 2.32 minutes (LC method A).

### Step 3: 4-Chloro-6-(2,6-dimethylphenyl)pyrimidin-2-amine (hydrochloride salt)

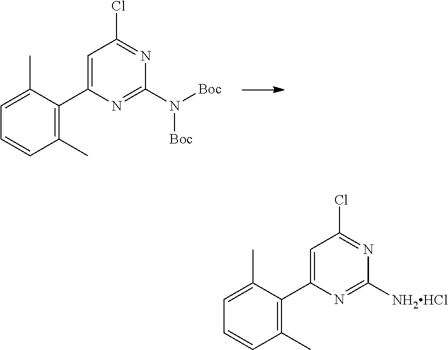

tert-Butyl N-tert-butoxycarbonyl-N-[4-chloro-6-(2,6-dimethylphenyl) pyrimidin-2-yl]carbamate (514.8 g, 915.9 mmol) was dissolved in dichloromethane (4 L). Hydrogen chloride in p-dioxane (1 L, 4 mol) was added and the mixture was stirred overnight at room temperature. The resulting precipitate was collected by vacuum filtration and dried in vacuo to obtain 4-chloro-6-(2,6-dimethylphenyl)pyrimidin-2-amine hydrochloride as a white solid (213.5 g, 82%). ¹H NMR (250 MHz, DMSO-d₆) δ 7.45-6.91 (m, 3H), 6.73 (s, 1H), 2.08 (s, 6H). ESI-MS m/z calc. 233.072, found 234.1 (M+1)⁺; Retention time: 2.1 minutes (LC Method C).

### Step 4: 4-Chloro-6-(2,6-dimethylphenyl)pyrimidin-2-amine

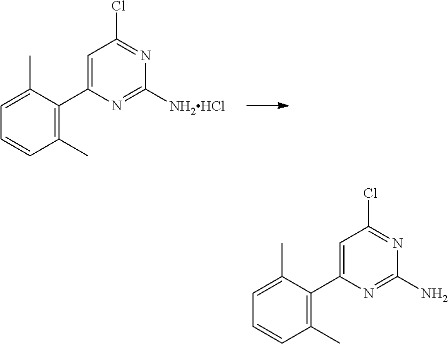

4-Chloro-6-(2,6-dimethylphenyl)pyrimidin-2-amine (hydrochloride salt) (166 g, 614.5 mmol) and 4-chloro-6-(2,6-dimethylphenyl)pyrimidin-2-amine (hydrochloride salt) (30 g, 111.0 mmol) were suspended in DCM (2.5 L), treated with NaOH (725 mL of 1 M, 725.0 mmol) and stirred at room temperature for 1 hour. The mixture was transferred into a separatory funnel and left standing over night. The DCM phase was separated and the aqueous phase with insoluble material was extracted twice more with DCM (2×500 mL). The combined brown DCM phases were stirred over magnesium sulfate and charcoal for 1 hour, filtered and the yellow solution concentrated to a volume of ˜ 500 mL. The solution was diluted with heptane (750 mL) and DCM was removed under reduced pressure at 60° C. to give a cream suspension. It was stirred at room temperature for 1 hour, filtered, washed with cold heptane and dried to give 4-chloro-6-(2,6-dimethylphenyl)pyrimidin-2-amine (157 g, 91%) as a cream solid. ¹H NMR (400 MHz, DMSO-d₆) δ 7.28-7.14 (m, 3H), 7.10 (d, J 7.5 Hz, 2H), 6.63 (s, 1H), 2.06 (s, 6H). ESI-MS m/z calc. 233.07198, found 234.0 (M+1)⁺; Retention time: 1.45 minutes (LC method A).

### Step 5: 3-[[4-Chloro-6-(2,6-dimethylphenyl)pyrimidin-2-yl]sulfamoyl]benzoic acid

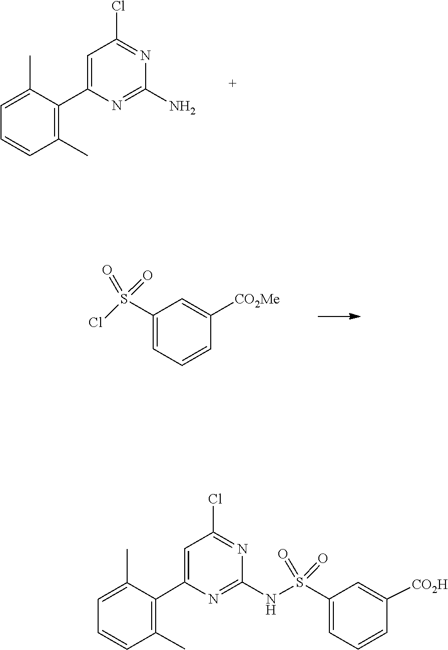 [SMILES: Cc1cccc(C)c1-c1cc(Cl)nc(N)n1 ; COC(=O)c1cccc(S(=O)(=O)Cl)c1 ; Cc1cccc(C)c1-c1cc(Cl)nc(NS(=O)(=O)c2cccc(C(=O)O)c2)n1]

4-Chloro-6-(2,6-dimethylphenyl)pyrimidin-2-amine (235 g, 985.5 mmol) was dissolved in MeTHF (2.3 L) and cooled in an ice bath under stirring and nitrogen. To the cold solution methyl 3-chlorosulfonylbenzoate (347 g, 1.479 mol) was added in one portion (seems slightly endothermic) and to the cold pale-yellow solution a solution of 2-methyl-butan-2-ol (Lithium salt) (875 mL of 3.1 M, 2.712 mol) (in heptane) was added dropwise over 1.25 hour (exothermic, internal temperature from 0 to 10° C.). The ice bath was removed and the greenish solution was stirred for 4 hours at room temperature. To the greenish solution cold HCl (2 L of 1.5 M, 3.000 mol) was added, the phases separated and the organic phase was washed once with water (1 L) and once with brine (500 mL). The aqueous phases were back extracted once with MeTHF (350 mL) and the organic phases were combined. This yellow MeTHF solution of methyl 3-[[4-chloro-6-(2,6-dimethylphenyl)pyrimidin-2-yl]sulfamoyl]benzoate (ESI-MS m/z calc. 431.07065, found 432.0 (M+1)⁺; Retention time: 1.81 minutes) was treated with NaOH (2.3 L of 2 M, 4.600 mol) and stirred at room temperature for 1 hour. The phases were separated and the NaOH phase was washed twice with MeTHF (2×500 mL) and the combined organic phases were extracted once with 2M NaOH (1×250 mL). The combined NaOH phases were combined, stirred in an ice bath and slowly acidified by addition of HCl (416 mL of 36% w/w, 4.929 mol) while keeping the internal temperature between 10 and 20° C. At the end of the addition (pH˜5-6) the final pH was adjusted to 2-3 by addition of solid citric acid. The formed yellow tacky suspension was stirred at room temperature overnight to give a cream crisp suspension. The solid was collected by filtration, washed with plenty of water and sucked dry for 3 hours. The solid was dried under reduced pressure with a nitrogen leak at 45-50° C. for 120 hours 3-[[4-chloro-6-(2,6-dimethylphenyl)pyrimidin-2-yl]sulfamoyl]benzoic acid (395 g, 96%) was isolated as an off-white solid. ¹H NMR (400 MHz, DMSO-d₆) δ 13.44 (s, 1H), 12.46 (s, 1H), 8.48-8.39 (m, 1H), 8.25-8.15 (m, 1H), 8.15-8.08 (m, 1H), 7.68 (t, J 7.8 Hz, 1H), 7.31 (s, 1H), 7.28-7.18 (m, 1H), 7.10 (d, J 7.6 Hz, 2H), 1.84 (s, 6H). ESI-MS m/z calc. 417.055, found 418.0 (M+1)⁺; Retention time: 1.56 minutes. (LC method A).

### Example B: Preparation of 3-[[4-[(2R)-2-(tert-Butoxycarbonylamino)-4-methyl-pentoxy]-6-(2,6-dimethylphenyl)pyrimidin-2-yl]sulfamoyl]benzoic acid

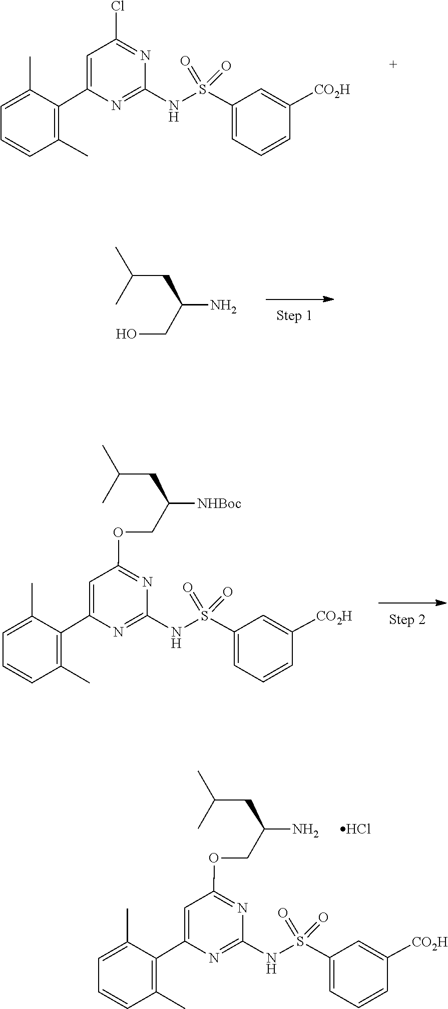

### Step 1: 3-[[4-[(2R)-2-(tert-Butoxycarbonylamino)-4-methyl-pentoxy]-6-(2,6-dimethylphenyl)pyrimidin-2-yl]sulfamoyl]benzoic acid

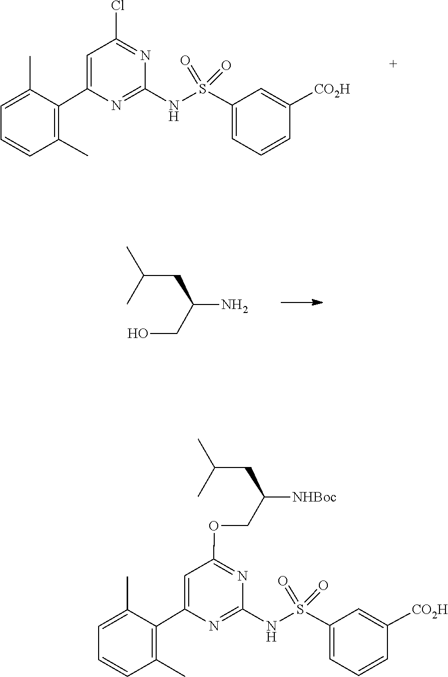

To a stirring solution of (2R)-2-amino-4-methyl-pentan-1-ol (12.419 g, 105.97 mmol) in anhydrous THE (200 mL) at room temperature under nitrogen was added sodium tert-butoxide (15.276 g, 158.95 mmol). The reaction mixture was stirred for 10 minutes and 3-[[4-chloro-6-(2,6-dimethylphenyl)pyrimidin-2-yl]sulfamoyl]benzoic acid (22.14 g, 52.983 mmol) was added. The reaction mixture was placed on a water bath preheated to 60° C. and stirred for 20 minutes. After cooling to room temperature, di-tert-butyl dicarbonate (69.381 g, 317.90 mmol) was added and the reaction mixture was stirred for 3 hours. The reaction was quenched with saturated aqueous ammonium chloride (150 mL). Volatiles were removed under vacuum and the aqueous layer was acidified to pH˜3 with 10% aqueous citric acid. The product was extracted with ethyl acetate (3×200 mL). The combined organic layers were washed with brine (80 mL), dried over anhydrous sodium sulfate and concentrated to a residual volume of ˜250 mL. The product was precipitated out into excess hexanes (750 mL) and collected by vacuum filtration. The obtained white solid was re-purified by silica gel chromatography using 0-40% acetone (0.15% acetic acid buffer) gradient in hexanes (0.15% acetic acid buffer) to afford 3-[[4-[(2R)-2-(tert-butoxycarbonylamino)-4-methyl-pentoxy]-6-(2,6-dimethylphenyl)pyrimidin-2-yl]sulfamoyl]benzoic acid (20.73 g, 61%) as a white solid. ESI-MS m/z calc. 598.2461, found 599.4 (M+1)⁺; Retention time: 5.85 minutes (LC Method S).

### Step 2: 3-[[4-[(2R)-2-Amino-4-methyl-pentoxy]-6-(2,6-dimethylphenyl)pyrimidin-2-yl]sulfamoyl]benzoic acid (hydrochloride salt)

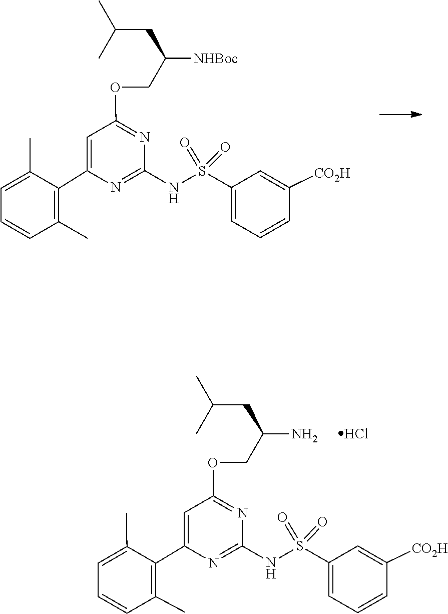 [SMILES: Cc1cccc(C)c1-c1cc(OC[C@@H](CC(C)C)NC(=O)OC(C)(C)C)nc(NS(=O)(=O)c2cccc(C(=O)O)c2)n1 ; Cc1cccc(C)c1-c1cc(OC[C@H](N)CC(C)C)nc(NS(=O)(=O)c2cccc(C(=O)O)c2)n1 ; Cl]

To a stirring solution of 3-[[4-[(2R)-2-(tert-butoxycarbonylamino)-4-methyl-pentoxy]-6-(2,6-dimethylphenyl)pyrimidin-2-yl]sulfamoyl]benzoic acid (20.73 g, 34.624 mmol) in DCM (200 mL) at room temperature was added HCl (87 mL of 4 M solution in 1,4-dioxane, 346.24 mmol). The reaction mixture was stirred for 2 hours. Volatiles were removed under vacuum and the obtained solid was triturated with diethyl ether (150 mL). After removal of the volatiles, the product was dried under vacuum to afford 3-[[4-[(2R)-2-amino-4-methyl-pentoxy]-6-(2,6-dimethylphenyl)pyrimidin-2-yl]sulfamoyl]benzoic acid (hydrochloride salt) (19.68 g, 100%) as a white solid. H NMR (250 MHz, DMSO-d₆) δ 8.56-8.27 (m, 4H), 8.14 (t, J 6.8 Hz, 2H), 7.70 (t, J 7.8 Hz, 1H), 7.34-7.18 (m, 1H), 7.17-7.02 (m, 2H), 6.31 (s, 1H), 4.42-4.23 (m, 1H), 4.23-4.06 (m, 1H), 3.5-3.4 (m, 1H, overlapped with water), 2.01 (s, 6H), 1.82-1.31 (m, 3H), 1.02-0.78 (m, 6H). ESI-MS m/z calc. 498.1937, found 499.3 (M+1)⁺; Retention time: 1.63 minutes (LC Method T).

### Example C: Preparation of N-[4-chloro-6-(2,6-dimethylphenyl)pyrimidin-2-yl]-3-nitro-benzenesulfonamide

### Step 1: N-[4-Chloro-6-(2,6-dimethylphenyl)pyrimidin-2-yl]-3-nitro-benzenesulfonamide

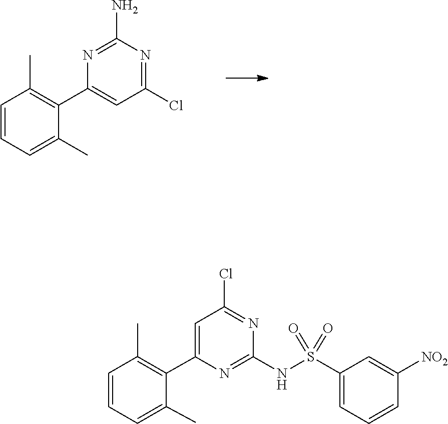 [SMILES: Cc1cccc(C)c1-c1cc(Cl)nc(N)n1 ; Cc1cccc(C)c1-c1cc(Cl)nc(NS(=O)(=O)c2cccc([N+](=O)[O-])c2)n1]

To a suspension of sodium hydride (60% in mineral oil) (4.87 g, 0.122 mol) in anhydrous tetrahydrofuran (30 mL) was added a solution of 4-chloro-6-(2,6-dimethylphenyl)pyrimidin-2-amine (8.13 g, 0.0348 mol) in anhydrous tetrahydrofuran (40 mL) dropwise at 0° C. The reaction mixture was stirred at room temperature for 30 minutes. A solution of 3-nitrobenzenesulfonyl chloride (11.57 g, 52.2 mmol) in anhydrous tetrahydrofuran (40 mL) was added to the reaction mixture dropwise at 0° C. The reaction was stirred at the same temperature for 1 hour. The reaction was quenched with a saturated aqueous solution of sodium bicarbonate (100 mL). The reaction solution was extracted with dichloromethane (3×100 mL). The combined organic layers were washed with water (100 mL), dried over anhydrous sodium sulfate, and then concentrated under vacuum. The residue was purified by silica gel column chromatography using 0 to 10% chloroform-ethyl acetate. The crude product was triturated with a solvent mixture of diethyl ether and hexane (1:5) to furnish N-[4-chloro-6-(2,6-dimethylphenyl)pyrimidin-2-yl]-3-nitro-benzenesulfonamide (5.98 g, 41%) as a white solid. ESI-MS m/z calc. 418.1, found 419.0 (M+1). Retention time: 5.73 minutes. ¹H NMR (250 MHz, CDCl₃) δ (ppm): 9.01 (s, 1H); 8.43 (t, J 10.5 Hz, 2H); 7.682 (t, J 7.8 Hz, 1H); 7.23 (m, 1H); 7.12 (d, J 7.5 Hz, 2H); 6.95 (s, 1H); 1.99 (s, 6H).

### Example D: Preparation of N-[4-(2,6-dimethylphenyl)-6-methylsulfonyl-pyrimidin-2-yl]-3-nitro-benzenesulfonamide

### Step 1: N-[4-(2,6-Dimethylphenyl)-6-methylsulfonyl-pyrimidin-2-yl]-3-nitro-benzenesulfonamide

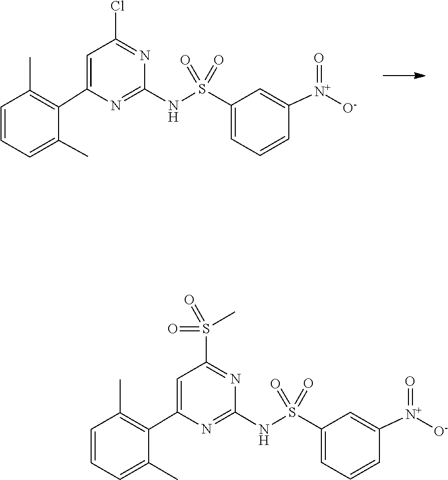 [SMILES: Cc1cccc(C)c1-c1cc(Cl)nc(NS(=O)(=O)c2cccc([N+](=O)[O-])c2)n1 ; Cc1cccc(C)c1-c1cc(S(C)(=O)=O)nc(NS(=O)(=O)c2cccc([N+](=O)[O-])c2)n1]

Stage 1: To a 250 mL round-bottomed flask were added N-[4-chloro-6-(2,6-dimethylphenyl)pyrimidin-2-yl]-3-nitro-benzenesulfonamide (14.14 g, 33.76 mmol), sodium thiomethoxide (5.86 g, 83.61 mmol) and NMP (130 mL). This solution was stirred at 100° C. for 3 h. The reaction mixture was then cooled to room temperature, quenched with 1 N HCl (300 mL), and extracted with ethyl acetate (3×300 mL). The combined organic extracts were washed with water (300 mL), 3% aqueous hydrogen peroxide solution (300 mL), water (300 mL) and saturated aqueous sodium chloride solution (300 mL), then dried over sodium sulfate, filtered, and evaporated in vacuo. This gave an orange foam (16.71 g, 115% crude product yield) that was carried onto the next reaction.

Stage 2: To a 250 mL round-bottomed flask containing the product from Stage 1, DCM (120 mL) was added, followed by m-CPBA (77% pure, 27.22 g, 121.5 mmol). This solution was stirred at room temperature for 90 min. The reaction mixture was quenched by transferring to a 1 L-Erlenmeyer flask containing DCM (400 mL) and solid Na₂S₂O₃ (41.15 g, 260.3 mmol). This mixture was stirred at room temperature for 1 h. The reaction mixture was diluted with DCM (300 mL), then washed with water (3×400 mL) and saturated aqueous sodium chloride solution (300 mL). The organic layer was then dried over sodium sulfate, filtered, and evaporated in vacuo. This solid was then partially dissolved in DCM (100 mL) and filtered in vacuo on a Buchner funnel to remove the m-chlorobenzoic acid waste (this was repeated three times). The remaining solution was then purified by silica gel chromatography (330 g of silica, 0 to 60% gradient of ethyl acetate/hexanes) to give N-[4-(2,6-dimethylphenyl)-6-methylsulfonyl-pyrimidin-2-yl]-3-nitro-benzenesulfonamide (5.881 g, 36%). ESI-MS m z calc. 462.06677, found 463.1 (M+1)⁺; Retention time: 1.6 minutes; LC method A.

### Example E: Preparation of 3-[[4-[(2R)-2-amino-4,4-dimethyl-pentoxy]-6-(2,6-dimethylphenyl)pyrimidin-2-yl]sulfamoyl]benzoic acid

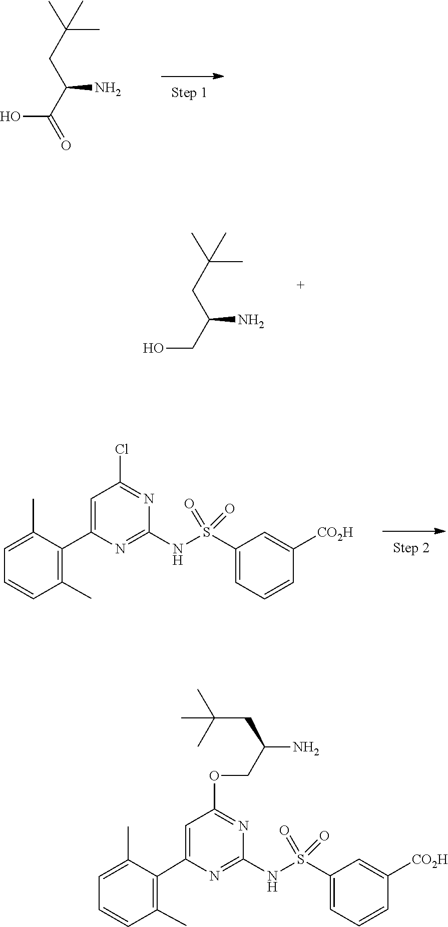

### Step 1: (2R)-2-Amino-4,4-dimethyl-pentan-1-ol

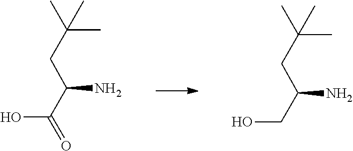

To a solution of (2R)-2-amino-4,4-dimethyl-pentanoic acid (15 g, 103.3 mmol) in THE (150 mL) at 0° C. was added borane-THF (260 mL of 1 M, 260.0 mmol) dropwise keeping the reaction temperature<10° C. The addition took approximately 30 min. The mixture was allowed to warm to ambient temperature and stirred for 22 h. The reaction was quenched with the slow addition of methanol (80 mL, 1.975 mol) and the solvent was removed in vacuo. The residue was co-evaporated 3× with methanol (200 mL, 4.937 mol) The crude residue was diluted with HCl (200 mL of 1 M, 200.0 mmol) and washed with 200 mL of MTBE. The aqueous phase was evaporated to remove residual organic solvent. The water was further removed in vacuo affording an off-white solid. The solid was further dried using an acetonitrile azeotrope. The solid was slurried in 200 mL of ACN and the precipitate collected using a M frit. The solid was air dried for 1 h, then in vacuo at 45° C. for 20 h to give (2R)-2-amino-4,4-dimethyl-pentan-1-ol (hydrochloride salt) (14.73 g, 85%). ¹H NMR (400 MHz, DMSO-d₆) δ 7.80 (s, 3H), 5.36 (t, J 5.1 Hz, 1H), 3.59 (dt, J 11.7, 4.1 Hz, 1H), 3.42-3.34 (m, 1H), 3.10 (dq, J 7.7, 3.8 Hz, 1H), 1.46 (dd, J 14.5, 7.1 Hz, 1H), 1.33 (dd, J 14.5, 3.5 Hz, 1H), 0.91 (s, 9H). ESI-MS m/z calc. 131.13101, found 132.1 (M+1)⁺; Retention time: 0.51 minutes (LC method A).

### Step 2: 3-[[4-[(2R)-2-Amino-4,4-dimethyl-pentoxy]-6-(2,6-dimethylphenyl)pyrimidin-2-yl]sulfamoyl]benzoic acid

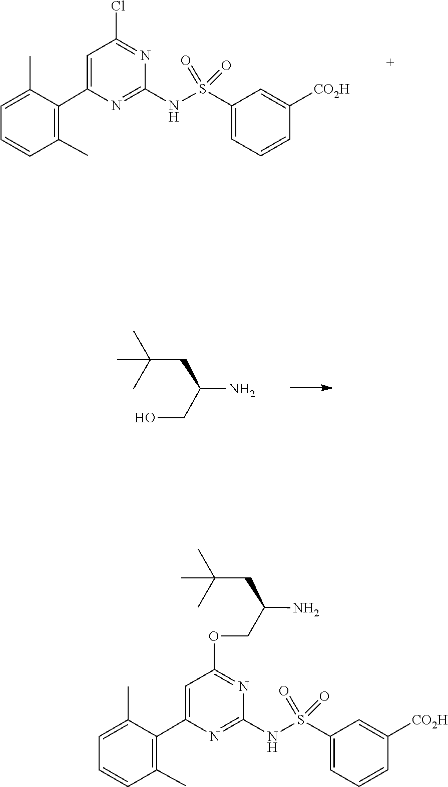

3-[[4-Chloro-6-(2,6-dimethylphenyl)pyrimidin-2-yl]sulfamoyl]benzoic acid (20 g, 47.862 mmol) was suspended in a mixture of 2-methyltetrahydrofuran (80 mL) and DMF (20 mL) and the solution was cooled to −5° C. Sodium tert-butoxide (23 g, 239.33 mmol) was then dissolved in 2-methyltetrahydrofuran (100 mL), cooled to 5° C. and added over 10 minutes, followed by (2R)-2-amino-4,4-dimethyl-pentan-1-ol (hydrochloride salt) (8.02 g, 47.830 mmol) the reaction was then warmed to 10° C. and stirred for 4 hours. It was then cooled to 0° C. and quenched by adding an aqueous solution of hydrochloric acid (2 M, 200 mL) over 10 minutes. The phases were separated, and the aqueous phase extracted with 2-methyltetrahydrofuran (200 mL). The organic phases were combined and washed with an aqueous solution of sodium chloride (15% w/w, 2×200 mL), dried over sodium sulfate (60 g), filtered and evaporated to dryness. The solid was then triturated using ethyl acetate (200 mL) for 16 hours, filtered, washed with ethyl acetate and dried in a vacuum oven at 50° C. for 20 hours to give 3-[[4-[(2R)-2-amino-4,4-dimethyl-pentoxy]-6-(2,6-dimethylphenyl)pyrimidin-2-yl]sulfamoyl]benzoic acid (hydrochloride salt) (22.29 g, 80%). ¹H NMR (400 MHz, DMSO-d₆) δ 13.26 (br. s., 2H), 8.45 (t, J 1.6 Hz, 1H), 8.28-8.06 (m, 5H), 7.69 (t, J 7.8 Hz, 1H), 7.31-7.21 (m, 1H), 7.13 (d, J 7.6 Hz, 2H), 6.29 (br. s., 1H), 4.30 (dd, J 11.7, 2.7 Hz, 1H), 4.10 (dd, J 11.5, 7.1 Hz, 1H), 3.56 (br. s., 1H), 2.13-1.90 (s, 6H), 1.62-1.47 (m, 2H), 0.94 (s, 9H). ESI-MS m/z calc. 512.20935, found 513.0 (M+1)⁺; Retention time: 2.334 minutes; LC method U.

### Example F: Preparation of 3-[[4-[(2R)-2-amino-5,5,5-trifluoro-4,4-dimethyl-pentoxy]-6-(2,6-dimethylphenyl)pyrimidin-2-yl]sulfamoyl]benzoic acid

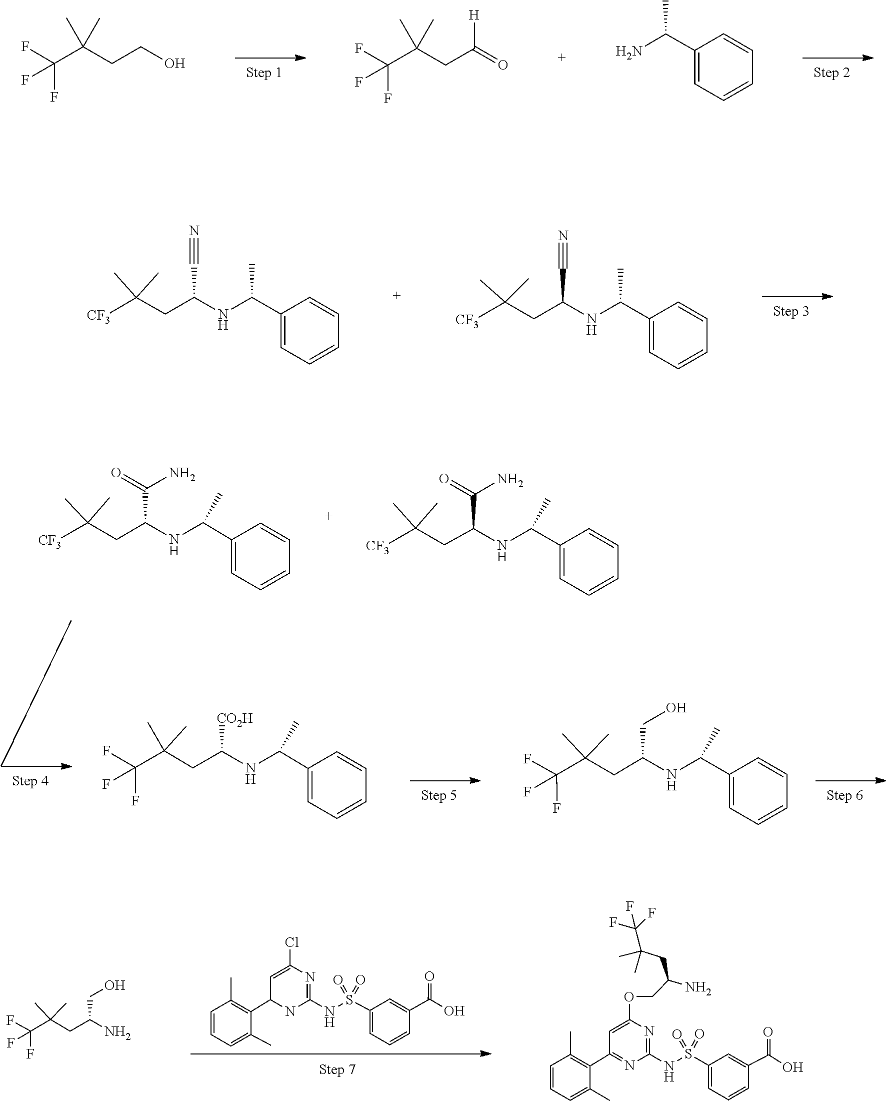

### Step 1: 4,4,4-Trifluoro-3,3-dimethyl-butanal

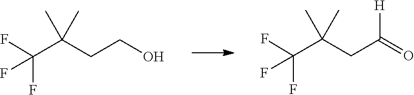

A 1 L three-neck flask was charged with 4,4,4-trifluoro-3,3-dimethyl-butan-1-ol (8.987 g, 57.555 mmol), DCM (63 mL), water (63 mL), NaBr (544 mg, 5.2870 mmol), sodium bicarbonate (12.32 g, 146.66 mmol) and TEMPO (92 mg, 0.5888 mmol). The mixture was cooled with ice-water bath. A aqueous solution of NaOCl (47 mL of 1.31 M, 61.570 mmol) was added dropwise over 2 h at 2.5-4.4° C. After the addition, the mixture was stirred for 10 min. The two layers was separated. The aqueous phase was extracted with DCM (2×15 mL). The combined organic layers were dried with sodium sulfate and filtered to give 113.7 g (about 80 mL) of crude product in DCM, which was used directly the next step. ¹H NMR (300 MHz, CDCl₃) δ 9.82-9.78 (m, 1H), 2.54 (d, J=2.6 Hz, 2H), 1.28 (s, 6H). ¹⁹F NMR (282 MHz, CDCl₃) δ-79.11 (s, 3F).

### Step 2: (2R)-5,5,5-Trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile

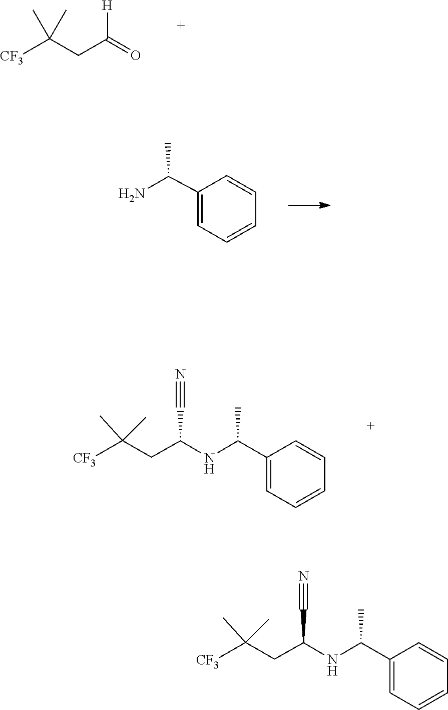

To a DCM (80 mL) solution of 4,4,4-trifluoro-3,3-dimethyl-butanal (113.7 g, 57.540 mmol) (purity about 7.8%) was added MeOH (110 mL). The mixture was cooled with ice-water bath. (1R)-1-phenylethanamine (8.46 g, 69.814 mmol) was added, followed by acetic acid (4.41 g, 73.436 mmol). The mixture was stirred at 0° C. for 10 min, then NaCN (3.56 g, 72.642 mmol) was added. The mixture was allowed to warm to rt slowly and stirred overnight. The reaction mixture was cooled to 0° C. and a solution of potassium carbonate (4 g) in water (20 mL) was added dropwise, followed by brine (40 mL). The mixture was extracted with DCM (2×100 mL). The organic layers were dried with sodium sulfate, filtered and concentrated. The residue was purified by flash chromatography (120 g silica gel, heptanes/EtOAc 0-30%) to afford a 4:1 mixture of (2R)-5,5,5-trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile (14.87 g, 91%) as a colorless oil. ESI-MS m/z calc. 284.15002, found 285.2 (M+1)⁺; Retention time: 3.38 minutes; LC method U.

### Step 3: (2R)-5,5,5-Trifluoro-4,4-dimethyl-2-[[(1R)-1-phenylethyl]amino]pentanamide and (2S)-5,5,5-trifluoro-4,4-dimethyl-2-[[(1R)-1-phenylethyl]amino]pentanamide

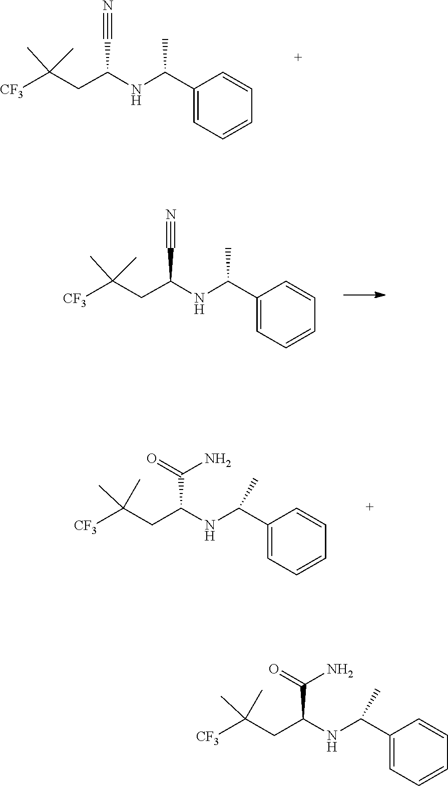 [SMILES: C[C@@H](N[C@@H](C#N)CC(C)(C)C(F)(F)F)c1ccccc1 ; C[C@@H](N[C@H](C#N)CC(C)(C)C(F)(F)F)c1ccccc1 ; C[C@@H](N[C@H](CC(C)(C)C(F)(F)F)C(N)=O)c1ccccc1 ; C[C@@H](N[C@@H](CC(C)(C)C(F)(F)F)C(N)=O)c1ccccc1]

To a solution of a 4:1 mixture of (2R)-5,5,5-trifluoro-4,4-dimethyl-2-[[(1R)-1-phenylethyl]amino]pentanenitrile and (2S)-5,5,5-trifluoro-4,4-dimethyl-2-[[(1R)-1-phenylethyl]amino]pentanenitrile (14.87 g, 52.300 mmol) in DCM (105 mL) was added sulfuric acid (56.3 g, 551.06 mmol). The mixture was stirred at rt overnight, poured on crude ice (200 g) and neutralized to pH 9 with 28% NH₃ in water (100 mL). The mixture was extracted with DCM (500 mL). The organic layer was dried with sodium sulfate, filtered and concentrated. The residue was purified by flash chromatography (330 g silica gel, heptanes/EtOAc 20-50%) to afford (2R)-5,5,5-trifluoro-4,4-dimethyl-2-[[(1R)-1-phenylethyl]amino]pentanamide (10.77 g, 68%) as a white solid. ¹H NMR (300 MHz, CDCl₃) δ 7.39-7.22 (m, 5H), 6.35 (br. s., 1H), 5.55 (br. s., 1H), 3.65 (q, J=6.5 Hz, 1H), 2.93 (dd, J=7.6, 3.8 Hz, 1H), 1.87 (dd, J=15.0, 3.8 Hz, 1H), 1.65-1.56 (m, 2H), 1.35 (d, J=6.5 Hz, 3H), 1.04 (s, 3H), 1.00 (s, 3H). ¹⁹F NMR (282 MHz, CDCl₃) δ-78.77 (s, 3F). 99.4% de by 19F NMR.

### Step 4: (2R)-5,5,5-Trifluoro-4,4-dimethyl-2-[[(1R)-1-phenylethyl]amino]pentanoic acid

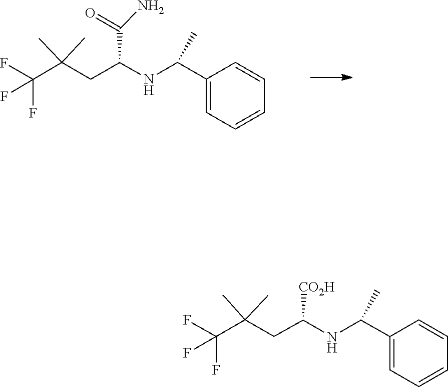 [SMILES: C[C@@H](N[C@H](CC(C)(C)C(F)(F)F)C(N)=O)c1ccccc1 ; C[C@@H](N[C@H](CC(C)(C)C(F)(F)F)C(=O)O)c1ccccc1]

To a solution of (2R)-5,5,5-trifluoro-4,4-dimethyl-2-[[(1R)-1-phenylethyl]amino]pentanamide (11.35 g, 37.541 mmol) in HOAc (50 mL) was added conc. HCl (65 mL of 11.8 M, 767.00 mmol), followed by water (50 mL). A white precipitate appeared. The mixture was heated at 100° C. for 66 h. More conc. HCl (40 mL of 11.8 M, 472.00 mmol) and HOAc (10 mL) were added. The mixture was stirred at 100° C. overnight. More HCl in water (20 mL of 6 M, 120.00 mmol) was added. After 7 h at 100° C., more HCl in water (20 mL of 6 M, 120.00 mmol) was added. The mixture was stirred at 100° C. overnight. It became a clear solution. More HCl in water (20 mL of 6 M, 120.00 mmol) was added. The mixture was stirred at 100° C. for 7 h, more HCl in water (20 mL of 6 M, 120.00 mmol) was added. The mixture was stirred at 100° C. overnight. The mixture was concentrated and co-evaporated with water (50 mL). The residue (17 g) was mixed with water (25 mL) at 50° C. for 20 min, cooled with ice-water bath for 20 min and filtered. The crude product was mixed with 1,4-dioxane (60 mL). The mixture was concentrated and dried on vacuum overnight to give (2R)-5,5,5-trifluoro-4,4-dimethyl-2-[[(1R)-1-phenylethyl]amino]pentanoic acid (hydrochloride salt) (13.04 g, 97%) as an off-white solid. ¹H NMR (300 MHz, DMSO-d₆) δ 10.09 (br. s., 1H), 7.54-7.31 (m, 5H), 7.29-7.05 (m, 1H), 4.07 (q, J=5.9 Hz, 1H), 3.16-2.98 (m, 1H), 2.08-1.83 (m, 2H), 1.49 (d, J=6.5 Hz, 3H), 0.99 (s, 3H), 0.92 (s, 3H). ¹⁹F NMR (282 MHz, DMSO-d₆) δ-78.28 (s, 3F). ESI-MS m/z calc. 303.14462, found 304.2 (M+1)⁺; Retention time: 1.98 minutes; LC method U.

### Step 5: (2R)-5,5,5-Trifluoro-4,4-dimethyl-2-[[(1R)-1-phenylethyl]amino]pentan-1-ol

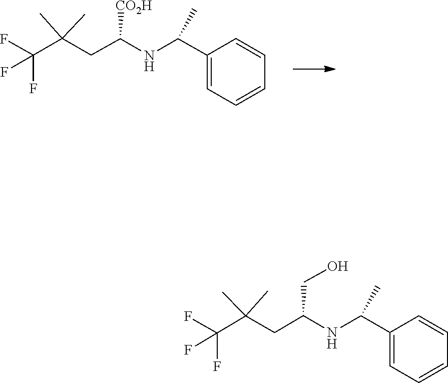 [SMILES: C[C@@H](N[C@H](CC(C)(C)C(F)(F)F)C(=O)O)c1ccccc1 ; C[C@@H](N[C@@H](CO)CC(C)(C)C(F)(F)F)c1ccccc1]

To a suspension of (2R)-5,5,5-trifluoro-4,4-dimethyl-2-[[(1R)-1-phenylethyl]amino]pentanoic acid (hydrochloride salt) (13.04 g, 36.267 mmol) in THE (200 mL) at 35° C. was added LAH in THE (100 mL of 1 M, 100.00 mmol) dropwise. The mixture was stirred at 40° C. for 2 h, cooled to 10° C. with ice-water bath and diluted with THE (200 mL). A mixture of water (3.8 g) and THE (50 mL) was added dropwise, followed by 25% aqueous NaOH (3.8 g) and water (10 g). The resulting mixture was stirred at rt for 30 min and at 50° C. for 1 h, filtered and washed with warm THF. The filtrate was concentrated to give 12.02 g of product (free amine) as a colorless oil. ¹H NMR (300 MHz, CDCl₃) δ 7.37-7.24 (m, 5H), 3.82 (q, J=6.5 Hz, 1H), 3.72-3.67 (m, 1H), 3.21 (dd, J=10.6, 4.7 Hz, 1H), 2.67 (quin, J=4.6 Hz, 1H), 1.66 (dd, J=14.7, 5.9 Hz, 1H), 1.54-1.45 (m, 1H), 1.36 (d, J=6.5 Hz, 3H), 1.03 (s, 3H), 0.97 (s, 3H). ¹⁹F NMR (282 MHz, CDCl₃) δ-78.83 (s, 3F). The above crude product (12.02 g) was dissolved in diethyl ether (20 mL) and diluted with heptanes (80 mL) and cooled in an ice-water bath. HCl in 1,4-dioxane (10.5 mL of 4 M, 42.000 mmol) was added dropwise. The mixture was stirred at rt for 30 min and filtered to give (2R)-5,5,5-trifluoro-4,4-dimethyl-2-[[(1R)-1-phenylethyl]amino]pentan-1-ol (hydrochloride salt) (11.56 g, 98%) as a white solid. ¹H NMR (300 MHz, DMSO-d₆) δ 9.57 (br. s., 1H), 9.25 (t, J=9.8 Hz, 1H), 7.80-7.59 (m, 2H), 7.53-7.32 (m, 3H), 5.63 (br. s., 1H), 4.58 (t, J=6.3 Hz, 1H), 3.81-3.65 (m, 1H), 3.64-3.51 (m, 1H), 2.91-2.74 (m, 1H), 1.98-1.85 (m, 1H), 1.85-1.74 (m, 1H), 1.63 (d, J=6.8 Hz, 3H), 0.91 (s, 3H), 0.88 (s, 3H). ¹⁹F NMR (282 MHz, DMSO-d₆) δ-77.71 (s, 3F).ESI-MS m/z calc. 289.16534, found 290.2 (M+1)⁺; Retention time: 2.08 minutes; LC method U.

### Step 6: (2R)-2-Amino-5,5,5-trifluoro-4,4-dimethyl-pentan-1-ol

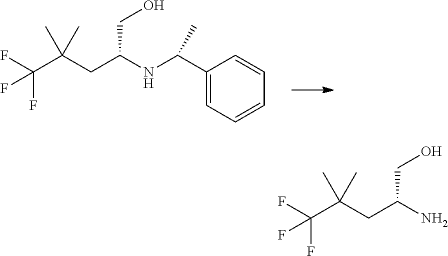 [SMILES: C[C@@H](N[C@@H](CO)CC(C)(C)C(F)(F)F)c1ccccc1 ; CC(C)(C[C@@H](N)CO)C(F)(F)F]

To a solution of (2R)-5,5,5-trifluoro-4,4-dimethyl-2-[[(1R)-1-phenylethyl]amino]pentan-1-ol (hydrochloride salt) (11.56 g, 35.482 mmol) in EtOH (200 mL) was added 10% palladium on carbon, 50% wet (5 g, 2.3492 mmol). The mixture was hydrogenated in a Parr shaker hydrogenation apparatus at 40 psi of hydrogen at rt for 9 h. More 10% palladium on carbon, 50% wet (1 g, 0.4698 mmol) was added. The mixture was shaken at 40 psi for 7 h. The mixture was filtered through Celite and washed with EtOH. The filtrate was concentrated. The residue (7.9 g) was triturated with a mixture of 2-methyltetrahydrofuran (28 mL) and heptanes (200 mL) and stirred overnight. The mixture was filtered, and the white solid was dried on vacuum to give (2R)-2-amino-5,5,5-trifluoro-4,4-dimethyl-pentan-1-ol (hydrochloride salt) (7.66 g, 93%) as a white solid. ¹H NMR (300 MHz, DMSO-d₆) δ 8.08 (br. s., 3H), 5.46 (t, J=5.0 Hz, 1H), 3.67-3.52 (m, 1H), 3.43 (dt, J=11.7, 5.8 Hz, 1H), 3.29-3.16 (m, 1H), 1.88-1.73 (m, 1H), 1.72-1.58 (m, 1H), 1.15 (s, 3H), 1.10 (s, 3H). ¹⁹F NMR (282 MHz, DMSO-d₆) δ-78.07 (s, 3F). ESI-MS m/z calc. 185.10275, found 186.2 (M+1)⁺; Retention time: 0.64 minutes; LC method U.

### Step 7: 3-[[4-[(2R)-2-Amino-5,5,5-trifluoro-4,4-dimethyl-pentoxy]-6-(2,6-dimethylphenyl)pyrimidin-2-yl]sulfamoyl]benzoic acid

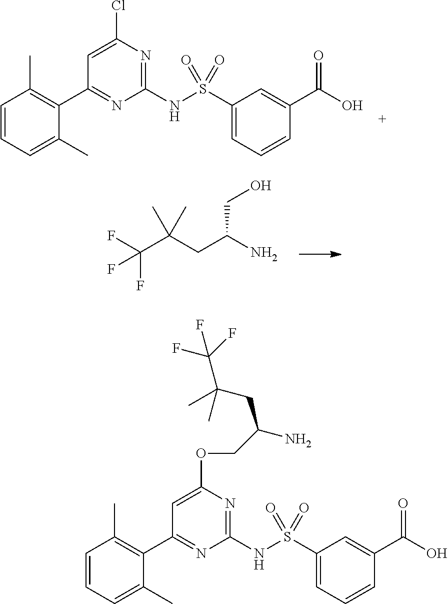

3-[[4-Chloro-6-(2,6-dimethylphenyl)pyrimidin-2-yl]sulfamoyl]benzoic acid (6.12 g, 14.65 mmol) and (2R)-2-amino-5,5,5-trifluoro-4,4-dimethyl-pentan-1-ol (hydrochloride salt) (3.27 g, 14.75 mmol) were combined in THE (30 mL) and the resulting suspension was cooled in a water-ice bath. Sodium tert-butoxide (5.63 g, 58.58 mmol) was added inducing rapid partial dissolution of the solid. After 5 minutes, the cooling bath was removed, and the reaction was stirred at room temperature for 1 hour (90% conversion). More (2R)-2-amino-5,5,5-trifluoro-4,4-dimethyl-pentan-1-ol (hydrochloride salt) (363 mg, 1.638 mmol) was added and the mixture was stirred for one hour (no change). More sodium tert-butoxide (744 mg, 7.742 mmol) was added and the mixture was stirred for 40 min (96% conversion). Ethyl acetate (100 mL), HCl (90 mL of 1 M, 90.00 mmol) and brine (50 mL) were added and the resulting two phases were separated. The organic phase was washed with brine (50 mL), dried over sodium sulfate and concentrated. The residue was triturated in EtOAc/MeOH/hexanes and the solvents were evaporated to give 3-[[4-[(2R)-2-amino-5,5,5-trifluoro-4,4-dimethyl-pentoxy]-6-(2,6-dimethylphenyl)pyrimidin-2-yl]sulfamoyl]benzoic acid (hydrochloride salt) (8.88 g, 93%) as a cream solid. ¹H NMR (400 MHz, DMSO-d₆) δ 13.15 (very broad s, 1H), 8.61-8.30 (m, 4H), 8.14 (dd, J 7.9, 1.9 Hz, 2H), 7.69 (t, J 7.8 Hz, 1H), 7.31-7.20 (m, 1H), 7.12 (d, J 7.6 Hz, 2H), 6.33 (s, 1H), 4.43 (dd, J 11.9, 3.3 Hz, 1H), 4.29-4.15 (m, 1H), 3.74 (s, 1H), 2.06-1.94 (broad m, 6H), 1.94-1.85 (m, 2H), 1.22 (s, 3H), 1.16 (s, 3H). ESI-MS m/z calc. 566.1811, found 567.62 (M+1)⁺; Retention time: 1.13 minutes (LC method A).

### Example G: Preparation of 3-[[4-[(2R)-2-amino-3-[1-(trifluoromethyl)cyclopropyl]propoxy]-6-(2,6-dimethylphenyl)pyrimidin-2-yl]sulfamoyl]benzoic acid

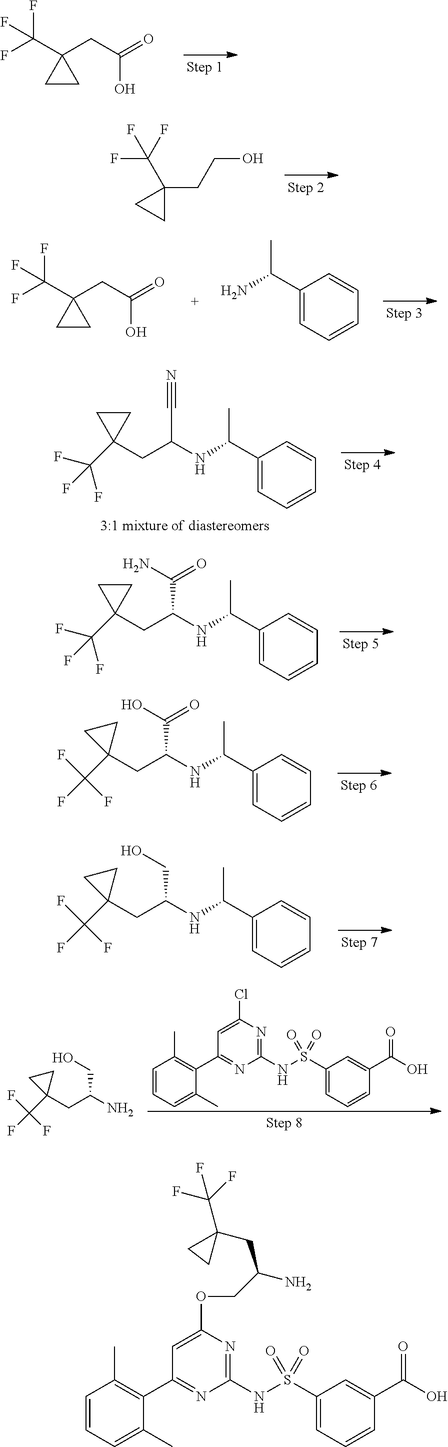

### Step 1: 2-[1-(Trifluoromethyl)cyclopropyl]ethanol

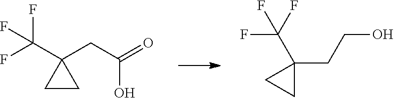 [SMILES: O=C(O)CC1(C(F)(F)F)CC1 ; OCCC1(C(F)(F)F)CC1]

LAH (49.868 g, 1.3139 mol) was added to THE (1700 mL) under nitrogen and the mixture was stirred for 30 minutes before being cooled to 0° C. 2-[1-(trifluoromethyl)cyclopropyl]acetic acid (190.91 g, 1.0107 mol) in THE (500 mL) was added dropwise while controlling the temperature<5° C. The mixture was allowed to warm up to room temperature and stirred for 24 hours. The resulting suspension was cooled to 0° C., water (50 mL) was added very slowly, followed by 15% w/w sodium hydroxide (50 mL) and water (150 mL). The mixture was stirred at 0° C. for 30 minutes, and filtered through Celite pad, the filter cake was washed with THE (2×500 mL). The combined filtrates were evaporated in vacuo to give 2-[1-(trifluoromethyl)cyclopropyl]ethanol (160.27 g, 98%) as amber oil containing˜5% w/w of THE (by NMR). ¹H NMR (250 MHz, DMSO-d₆) δ 4.57 (t, J 5.2 Hz, 1H), 3.55-3.39 (m, 2H), 1.74 (t, J 7.3 Hz, 2H), 1.00-0.58 (m, 4H).

### Step 2: 2-[1-(Trifluoromethyl)cyclopropyl]acetaldehyde

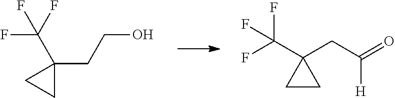 [SMILES: OCCC1(C(F)(F)F)CC1 ; O=CCC1(C(F)(F)F)CC1]

To a solution of 2-[1-(trifluoromethyl)cyclopropyl]ethanol (80 g, 467.1 mmol) in methylene chloride (1.1 L) was stirred at room temperature and treated with Dess-Martin periodinane (250 g, 589.4 mmol) portionwise (exothermic! cooled in ice bath and kept T<15° C.). To the mixture was added water (12 mL, 666.1 mmol) slowly added over 0.5 h (exothermic during addition up to 33° C., kept between 20 and 33° C. by cooling with cold water) giving a thick suspension. After the addition, the pale-yellow fine suspension was stirred at room temperature for 18 h. The yellow suspension was diluted with diethylether (500 mL) (yellow suspension) and stirred for 30 min. The slurry was filtered over Celite and the precipitate washed with 100 mL of Diethylether. diethylether. The organic phase was carefully treated with a saturated aqueous solution of sodium carbonate (500 ml, strong gas evolution, pH˜10 at the end). The three-phase mixture was stirred at room temperature for 1 h and the solid was removed by filtration (large glass frit). The phases (yellow cloudy Diethylether phase, colorless water phase) were separated and the organic phase was washed once more with a saturated aqueous solution of sodium carbonate (250 mL), once with 1M sodium thiosulfate (250 mL) and once with brine (250 mL). The aqueous phases were back extracted once with diethyl ether (150 mL) and the combined organic phases were dried, filtered and evaporated to give 2-[1-(trifluoromethyl)cyclopropyl]acetaldehyde (40 g, 56%) as a yellow liquid.

### Step 3: 2-[[(1R)-1-Phenylethyl]amino]-3-[1-(trifluoromethyl)cyclopropyl]propanenitrile

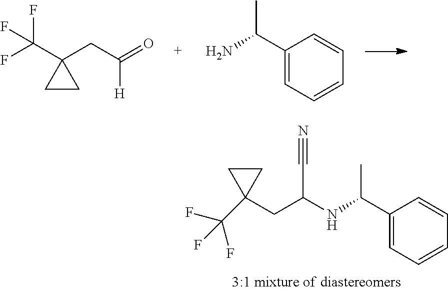

2-[1-(Trifluoromethyl)cyclopropyl]acetaldehyde (102 g, 670.5 mmol) in MeOH (700 mL) was treated with (1R)-1-phenylethanamine (86 mL, 667.1 mmol) and cooled in an ice bath. The solution was treated with acetic acid (38 mL, 668.2 mmol), stirred for 20 min in the ice bath, then solid NaCN (CAUTION, 33 g, 673.4 mmol) was added in one portion and the suspension was stirred in the melting ice bath for 14 hours. The solution was concentrated under reduced pressure (CAUTION, HCN!, the exhaust from the pump was running through a bleach trap) and the residue was extracted with MTBE (1000 mL) and saturated sodium carbonate/water 1:1 (1000 mL) and washed with brine (350 mL). The aqueous phases were back extracted once with MTBE (250 mL) and the combined organic phases were dried, filtered and evaporated to give 2-[[(1R)-1-phenylethyl]amino]-3-[1-(trifluoromethyl)cyclopropyl]propanenitrile (180.8 g, 96%) as 3:1 mixture of diastereomers. ESI-MS m/z calc. 282.13437, found 283.0 (M+1)⁺; Retention time: 1.69 minutes (major isomer) and 1.62 minutes (minor isomer), LC method A.

### Step 4: (2R)-2-[[(1R)-1-Phenylethyl]amino]-3-[1-(trifluoromethyl)cyclopropyl]propenamide

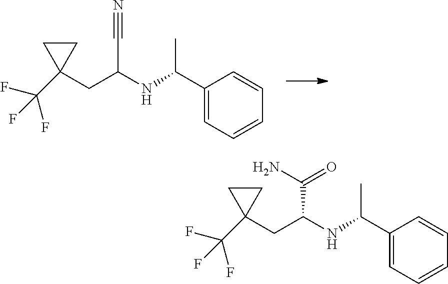

In a 2 L flask equipped with mechanical stirring and a temperature probe, sulfuric acid (285 mL of 18 M, 5.130 mol) was added it was cooled in an ice bath. At an internal temperature of 5° C., a solution of 2-[[(1R)-1-phenylethyl]amino]-3-[1-(trifluoromethyl)cyclopropyl]propanenitrile (180.8 g, 640.4 mmol, 3:1 mixture of diastereomers) in DCM (900 mL) was added dropwise over 20 minutes. The ice bath was removed, and the deep orange emulsion was stirred at room temperature for 18 h and at 30-40° C. for 2 h. The deep orange emulsion was carefully added to a mixture of ice and water (2.2 L) under mechanical stirring to give a yellow three phase mixture which was basified by slow addition of ammonium hydroxide (1.33 L of 30% w/w, 10.25 mol) under ice cooling (very exothermic, internal temperature kept between 10 and 25° C. by adding ice). The yellow emulsion was stirred for 10 minutes at room temperature (pH˜10), diluted with DCM (500 mL) and the phases were separated. The aqueous phase was washed twice more with DCM (400 and 200 mL) and the combined organic phases were washed once with water/brine 1:1 (500 mL). The DCM phase was dried, filtered and evaporated to give crude 2-[[(1R)-1-phenylethyl]amino]-3-[1-(trifluoromethyl)cyclopropyl]propanamide (189.5 g, 99%) as a yellow-orange oil. ESI-MS m z calc. 300.14496, found 301.0 (M+1)⁺; Retention time: 1.40 minutes (major isomer) and 1.50 minutes (minor isomer) (3:1 mixture of diastereomers). The product was dissolved in ethanol (1.5 L) and it was treated quickly with HCl (240 mL of 4 M, 960.0 mmol) (4M in dioxane) and the resulting thick suspension was stirred at room temperature overnight under mechanic stirring. The solid was collected by filtration, washed with cold ethanol and dried under vacuum with a nitrogen bleed at 40-45° C. to give (2R)-2-[[(1R)-1-phenylethyl]amino]-3-[1-(trifluoromethyl)cyclopropyl]propanamide (hydrochloride salt) (147 g, 68%). ¹H NMR (499 MHz, DMSO-d₆) δ 9.74 (d, J=67.9 Hz, 2H), 8.16-7.94 (m, 1H), 7.86 (s, 1H), 7.64-7.51 (m, 2H), 7.51-7.34 (m, 3H), 4.22 (s, 1H), 3.46-3.37 (m, 1H), 2.45 (d, J=15.9 Hz, 1H), 1.85 (dd, J 15.1, 10.4 Hz, 1H), 1.58 (d, J 6.7 Hz, 3H), 0.89 (pd, J 9.6, 9.2, 4.3 Hz, 2H), 0.84-0.66 (m, 2H). ESI-MS m/z calc. 300.14496, found 301.0 (M+1)⁺; Retention time: 1.40 minutes (major isomer) and 1.40 minutes (minor isomer), 97:3 mixture of diastereomers (LC method V).

### Step 5: (2R)-2-[[(1R)-1-Phenylethyl]amino]-3-[1-(trifluoromethyl)cyclopropyl]propanoic acid

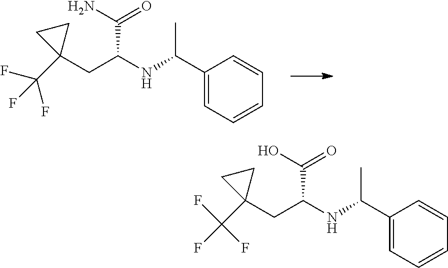

In a 5 L flask equipped with mechanical stirring, (2R)-2-[[(1R)-1-phenylethyl]amino]-3-[1-(trifluoromethyl)cyclopropyl]propanamide (hydrochloride salt) (147 g, 436.5 mmol) was added to acetic acid (735 mL) under stirring and the thick colorless suspension was treated with HCl (1.3 L of 12 M, 15.60 mol). The colorless suspension was carefully heated to 60-65° C. (strong foaming, acetic acid (145 mL) was added) and the suspension was stirred at 60-65° C. for 16 h. The suspension was then slowly heated to 100° C. (over 4 h, strong foaming) and the resulting solution was stirred at 100° C. for another 20 h. The pale-yellow solution was concentrated under reduced pressure at 65° C. to a semisolid mass and it was treated with water (1.5 L). The thick suspension was heated to 70-80° C. and left to cool to room temperature under stirring for 2 h. The solid was collected by filtration, washed with water and sucked dry overnight. The wet solid was further dried under reduced pressure at 50-60° C. for 4 h to give (2R)-2-[[(1R)-1-phenylethyl]amino]-3-[1-(trifluoromethyl)cyclopropyl]propanoic acid (hydrochloride salt) (135 g, 92%) as an off-white solid. ESI-MS m/z calc. 301.12897, found 302.0 (M+1)⁺; Retention time: 1.82 minutes; (LC method V).

### Step 6: (2R)-2-[[(1R)-1-phenylethyl]amino]-3-[1-(trifluoromethyl)cyclopropyl]propan-1-ol

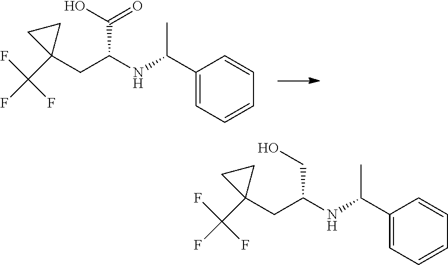 [SMILES: C[C@@H](N[C@H](CC1(C(F)(F)F)CC1)C(=O)O)c1ccccc1 ; C[C@@H](N[C@@H](CO)CC1(C(F)(F)F)CC1)c1ccccc1]

In a 5 L flask equipped with mechanical stirring and under dry nitrogen atmosphere, (2R)-2-[[(1R)-1-phenylethyl]amino]-3-[1-(trifluoromethyl)cyclopropyl]propanoic acid (hydrochloride salt) (135 g, 399.7 mmol) was suspended in THE (2 L) (thick suspension). It was heated to 35-40° C. and LAH (47.3 g, 1.214 mol) (pellets) was slowly added over 1 hour, while keeping the internal temperature between 30 and 40° C. by external cooling. The mixture was stirred for 1 hour at 30-40° C. (almost no hydrogen evolution anymore, grey suspension, most starting material in solution) and it was heated at 50-55° C. for 1 h. The grey suspension was left stirring in the cooling heating mantel overnight. The grey suspension was cooled in an ice bath and quenched by careful addition of water (44 mL, 2.442 mol), NaOH (41 mL of 6 M, 246.0 mmol) and water (44 mL, 2.442 mol) (high exotherm with first water addition, kept between 5° C. and 30° C. by cooling). The grey suspension was heated to 50-55° C. for 1 h, by which time a colorless suspension was obtained. The warm suspension was filtered over a pad of Celite covered over magnesium sulfate. The solids were washed with hot THF and evaporated to give crude (2R)-2-[[(1R)-1-phenylethyl]amino]-3-[1-(trifluoromethyl)cyclopropyl]propan-1-ol (121 g, 105%) as an oil. The crude was dissolved in diethyl ether (1 L, clear solution) and slowly treated with HCl (101 mL of 4 M, 404.0 mmol) (4M in dioxane) under cooling. The resulting thick suspension was stirred at room temperature for 1 h, the solid collected by filtration, washed with diethyl ether and dried under reduced pressure at 40-45° C. with a nitrogen bleed to give (2R)-2-[[(1R)-1-phenylethyl]amino]-3-[1-(trifluoromethyl)cyclopropyl]propan-1-ol (hydrochloride salt) (126.6 g, 98%) as an off-white solid. ¹H NMR (500 MHz, DMSO-d₆) δ 9.34 (s, 2H), 7.66 (d, J 7.4 Hz, 2H), 7.43 (dt, J 25.1, 7.4 Hz, 3H), 5.59 (s, 1H), 4.58 (q, J 6.6 Hz, 1H), 3.83 (d, J=12.6 Hz, 1H), 3.62-3.54 (m, 1H), 2.89 (s, 1H), 2.33-2.24 (m, 1H), 1.67-1.51 (m, 4H), 0.97-0.81 (m, 3H), 0.71 (s, 1H). ESI-MS m/z calc. 287.1497, found 288.0 (M+1)⁺; Retention time: 0.99 minutes (LC method A).

### Step 7: (2R)-2-Amino-3-[1-(trifluoromethyl)cyclopropyl]propan-1-ol

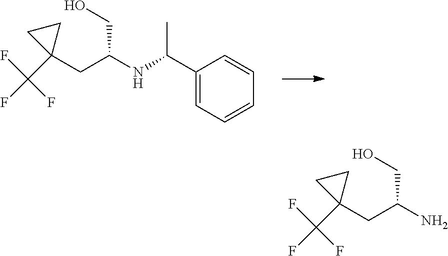 [SMILES: C[C@@H](N[C@@H](CO)CC1(C(F)(F)F)CC1)c1ccccc1 ; N[C@@H](CO)CC1(C(F)(F)F)CC1]

In a 1 L hydrogenation reactor, (2R)-2-[[(1R)-1-phenylethyl]amino]-3-[1-(trifluoromethyl)cyclopropyl]propan-1-ol (hydrochloride salt) (63.3 g, 195.5 mmol) was dissolved in EtOH (630 mL) (under warming), and it was treated with Pd/C (6.3 g of 10% w/w, 5.920 mmol) (12.5 g of 50% water wet) and the reaction was stirred under 2 bar of hydrogen at 40° C. for 24 h. The reaction mixture was filtered over Celite. The pad was washed with ethanol and the colorless filtrate was evaporated to a solid mass, which was triturated with diethyl ether. The suspension was stirred at room temperature for 1 h. The solid was filtered, washed with plenty of diethyl ether and dried to give (2R)-2-amino-3-[1-(trifluoromethyl)cyclopropyl]propan-1-ol (hydrochloride salt) (41.8 g, 97%) as an off-white solid. ¹H NMR (500 MHz, DMSO-d₆) δ 8.18 (s, 3H), 5.45 (t, J 4.9 Hz, 1H), 3.71 (dt, J 11.6, 3.9 Hz, 1H), 3.55 (dt, J 11.2, 5.4 Hz, 1H), 3.24 (h, J 4.7 Hz, 1H), 2.08 (dd, J 15.1, 5.4 Hz, 1H), 1.69 (dd, J 15.1, 9.4 Hz, 1H), 0.97 (h, J 6.5, 5.9 Hz, 2H), 0.86 (s, 2H). ESI-MS m z calc. 183.0871, found 184.0 (M+1)⁺; Retention time: 0.65 minutes; LC method A.

### Step 8: 3-[[4-[(2R)-2-Amino-3-[1-(trifluoromethyl)cyclopropyl]propoxy]-6-(2,6-dimethylphenyl)pyrimidin-2-yl]sulfamoyl]benzoic acid

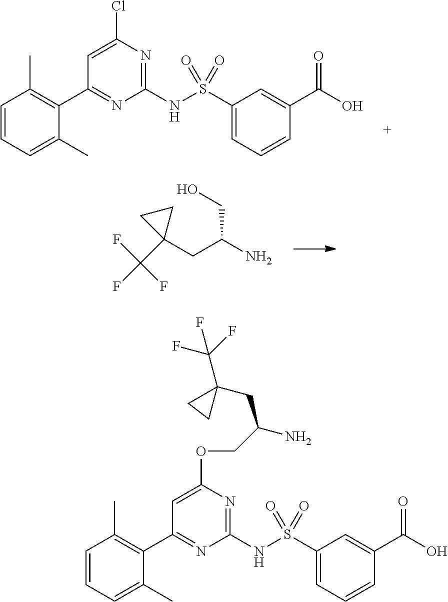 [SMILES: Cc1cccc(C)c1-c1cc(Cl)nc(NS(=O)(=O)c2cccc(C(=O)O)c2)n1 ; N[C@@H](CO)CC1(C(F)(F)F)CC1 ; Cc1cccc(C)c1-c1cc(OC[C@H](N)CC2(C(F)(F)F)CC2)nc(NS(=O)(=O)c2cccc(C(=O)O)c2)n1]

3-[[4-Chloro-6-(2,6-dimethylphenyl)pyrimidin-2-yl]sulfamoyl]benzoic acid (19.09 g, 45.68 mmol) and (2R)-2-amino-3-[1-(trifluoromethyl)cyclopropyl]propan-1-ol (hydrochloride salt) (10.18 g, 46.35 mmol) were dissolved in THE (100 mL) and cooled in an ice water bath. Sodium tert-butoxide (18.14 g, 188.8 mmol) was added and the reaction was allowed to warm to room temperature. The reaction was stirred for 1 h, then partitioned between ethyl acetate (500 mL) and aqueous HCl (275 mL of 1 M, 275.0 mmol). The organics were separated, washed with brine, dried over sodium sulfate and evaporated to give 3-[[4-[(2R)-2-amino-3-[1-(trifluoromethyl)cyclopropyl]propoxy]-6-(2,6-dimethylphenyl)pyrimidin-2-yl]sulfamoyl]benzoic acid (hydrochloride salt) (26.74 g, 94%). ESI-MS m/z calc. 564.1654, found 565.1 (M+1)⁺; Retention time: 0.48 minutes; LC method D.

### Example H: Preparation of 3-[1-(trifluoromethyl)cyclopropyl]propan-1-ol

### Step 1: 2-[1-(Trifluoromethyl)cyclopropyl]ethyl methanesulfonate

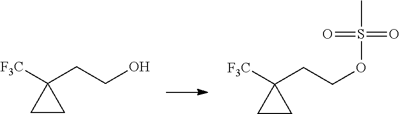

A 1000 mL, 3-neck round bottom flask was fitted with a mechanical stirrer, a cooling bath, a J-Kem temperature probe, an addition funnel and a nitrogen inlet/outlet. The vessel was charged under a nitrogen atmosphere with 2-[1-(trifluoromethyl)cyclopropyl]ethanol (125 g, 811.0 mmol) and 2-methyltetrahydrofuran (625 mL) which provided a clear colorless solution. Stirring was commenced and the pot temperature was recorded at 19° C. The vessel was then charged with triethylamine (124.3 mL, 891.8 mmol) added neat in one portion. The cooling bath was then charged with crushed ice/water and the pot temperature was lowered to 0° C. The addition funnel was charged with a solution of methanesulfonyl chloride (62.77 mL, 811.0 mmol) in 2-methyltetrahydrofuran (125 mL, 2 mL/g) which was subsequently added dropwise over 90 min which resulted in a white suspension and an exotherm to 1° C. The mixture was allowed to slowly warm to room temperature and continue to stir at room temperature for 1 h at which point the mixture was poured into ice cold water (250 mL) and then transferred to a separatory funnel. The organic was removed and washed with 20 wt % potassium bicarbonate solution (250 mL), dried over sodium sulfate (200 g) and then filtered through a glass frit Buchner funnel. The clear filtrate was concentrated under reduced pressure to provide 2-[1-(trifluoromethyl)cyclopropyl]ethyl methanesulfonate (185 g, 98%) as a clear pale yellow oil. ¹H NMR (400 MHz, Chloroform-d) δ 4.36 (ddt, J 7.1, 6.4, 0.7 Hz, 2H), 3.02 (s, 3H), 2.03 (t, J 7.1 Hz, 2H), 1.11-0.98 (m, 2H), 0.81-0.66 (m, 2H).

### Step 2: 3-[1-(Trifluoromethyl)cyclopropyl]propanenitrile

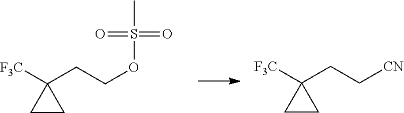

A 1000 mL, 3-neck round bottom flask was fitted with a mechanical stirrer, a heating mantle, a J-Kem temperature probe/controller, a water cooled reflux condenser and a nitrogen inlet/outlet. The vessel was charged under a nitrogen atmosphere with 2-[1-(trifluoromethyl)cyclopropyl]ethyl methanesulfonate (50 g, 215.3 mmol) and dimethyl sulfoxide (250 mL) which provided a clear pale yellow solution. Stirring was commenced and the pot temperature was recorded at 19° C. The vessel was charged with sodium cyanide (13.19 g, 269.1 mmol), added as a solid in one portion. The mixture was heated to a pot temperature of 70° C. and the condition was maintained for 24 h. Upon heating all of the sodium cyanide dissolved and the reaction mixture turned to a light amber suspension. After cooling to room temperature, the reaction mixture was poured into water (500 mL) and then transferred to a separatory funnel and partitioned with methyl tert-butyl ether (500 mL). The organic was removed and the residual aqueous was extracted with methyl tert-butyl ether (3×250 mL). The combined organic layers were washed with water (2×250 mL), dried over sodium sulfate (200 g) and then filtered through a glass frit Buchner funnel. The clear filtrate was concentrated under reduced pressure to provide 3-[1-(trifluoromethyl)cyclopropyl]propanenitrile (30 g, 85%) as a clear amber oil. ¹H NMR (400 MHz, Chloroform-d) δ 2.55 (t, J 7.6 Hz, 2H), 1.93 (t, J 7.7 Hz, 2H), 1.11-1.04 (m, 2H), 0.78-0.70 (m, 2H).

### Step 3: 3-[1-(Trifluoromethyl)cyclopropyl]propanoic acid

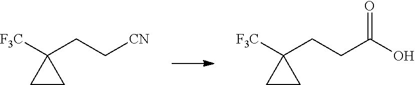 [SMILES: N#CCCC1(C(F)(F)F)CC1 ; O=C(O)CCC1(C(F)(F)F)CC1]

A 1000 mL, 3-neck round bottom flask was fitted with a mechanical stirrer, a heating mantle, a J-Kem temperature probe/controller, a water cooled reflux condenser and a nitrogen inlet/outlet. The vessel was subsequently charged under a nitrogen atmosphere with 3-[1-(trifluoromethyl)cyclopropyl]propanenitrile (25 g, 153.2 mmol) and ethyl alcohol (375 mL) which provided a clear amber solution. Stirring was commenced and the pot temperature was recorded at 19° C. The vessel was then charged with sodium hydroxide (102.1 mL of 6 M, 612.6 mmol), added in one portion. The resulting clear amber solution was heated to a pot temperature of 70° C. and the condition was maintained for 24 h. After cooling to room temperature, the reaction mixture was concentrated to remove the ethyl alcohol. The residual aqueous was diluted with water (150 mL) and then transferred to a separatory funnel and partitioned with methyl tert-butyl ether (50 mL). The aqueous was removed and the pH was adjusted to pH˜ 1 with 6 M hydrochloric acid solution. The resulting aqueous solution was transferred to a separatory funnel and partitioned with methyl tert-butyl ether (250 mL). The organic was removed and the residual aqueous was extracted with methyl tert-butyl ether (2×150 mL). The combined organic was dried over sodium sulfate (150 g) and then filtered through a glass frit Buchner funnel. The clear filtrate was concentrated under reduced pressure to provide 3-[1-(trifluoromethyl)cyclopropyl]propanoic acid (26 g, 93%) as a clear amber oil. ¹H NMR (400 MHz, Chloroform-d) δ 2.63-2.50 (m, 2H), 1.96-1.84 (m, 2H), 1.03-0.95 (m, 2H), 0.66-0.58 (m, J 1.7 Hz, 2H).

### Step 4: 3-[1-(Trifluoromethyl)cyclopropyl]propan-1-ol

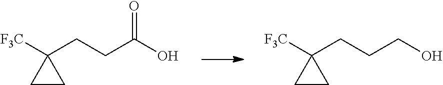 [SMILES: O=C(O)CCC1(C(F)(F)F)CC1 ; OCCCC1(C(F)(F)F)CC1]

A 1000 mL, 3-neck round bottom flask was fitted with a mechanical stirrer, a cooling bath, an addition funnel, a J-Kem temperature probe and a nitrogen inlet/outlet. The vessel was charged under a nitrogen atmosphere with lithium aluminum hydride pellets (6.775 g, 178.5 mmol). The vessel was then charged under a nitrogen atmosphere with tetrahydrofuran (250 mL). Stirring was commenced and the pot temperature was recorded at 20° C. The mixture was allowed to stir at room temperature for 0.5 h to allow the pellets to dissolve. The pot temperature of the resulting grey suspension was recorded at 24° C. The cooling bath was then charged with crushed ice/water and the pot temperature was lowered to 0° C. The addition funnel was charged with a solution of 3-[1-(trifluoromethyl)cyclopropyl]propanoic acid (25 g, 137.3 mmol) in tetrahydrofuran (75 mL, 3 mL/g) and the clear pale yellow solution was added dropwise over 1 h. After the addition was completed, the pot temperature of the resulting greyish-brown suspension was recorded at 5° C. The mixture was allowed to slowly warm to room temperature and continue to stir at room temperature for 24 h. The suspension was cooled to 0° C. with a crushed ice/water cooling bath and then quenched by the very slow dropwise addition of water (6.775 mL), followed by 15 wt % sodium hydroxide solution (6.775 mL) and then finally with water (20.32 mL). The pot temperature of the resulting white suspension was recorded at 5° C. The suspension was continued to stir at ˜5° C. for 30 min and then filtered through a glass frit Buchner funnel with a 20 mm layer of celite. The filter cake was displacement washed with tetrahydrofuran (2×150 mL) and then dried under vacuum for 15 min. The filtrate was dried over sodium sulfate (250 g) and then filtered through a glass frit Buchner funnel. The filtrate was concentrated under reduced pressure to provide a clear pale amber oil as the desired product, 3-[1-(trifluoromethyl)cyclopropyl]propan-1-ol (21.2 g, 92%). ¹H NMR (400 MHz, Chloroform-d) δ 3.65 (t, J 6.0 Hz, 2H), 1.78-1.59 (m, 4H), 0.99-0.91 (m, 2H), 0.59 (dp, J 4.7, 1.7 Hz, 2H).

### Example I: Preparation of 6-[[4-chloro-6-(2,6-dimethylphenyl)pyrimidin-2-yl]sulfamoyl]pyridine-2-carboxylic acid

### Step 1: Methyl 6-benzylsulfanylpyridine-2-carboxylate

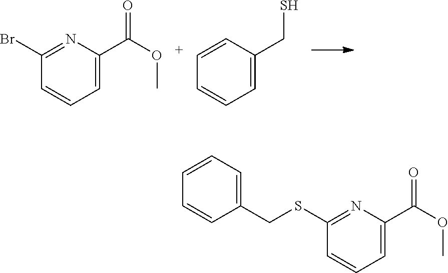

To a solution of phenylmethanethiol (28.408 g, 26.800 mL, 228.72 mmol) in THE (600 mL) was added NaH (11.200 g, 60% w/w, 280.03 mmol) in a few portions at 0° C. The slurry was warmed to room temperature and stirred for 30 min, then methyl 6-bromopyridine-2-carboxylate (50 g, 231.45 mmol) was added as a single portion. After 3 h, the reaction was diluted with ether (800 mL) and quenched with water (400 mL) and saturated sodium bicarbonate (50 mL). The layers were separated, and the organic layer was washed with brine, dried over sodium sulfate, and concentrated under reduced pressure to yield methyl 6-benzylsulfanylpyridine-2-carboxylate (56.35 g, 89%) as a yellow oil. ¹H NMR (500 MHz, DMSO-d₆) δ 7.84-7.77 (m, 1H), 7.77-7.73 (m, 1H), 7.52 (m, 1H), 7.48 (d, J 7.8 Hz, 2H), 7.28 (t, J 7.2, 7.2 Hz, 2H), 7.24-7.18 (m, 1H), 4.44 (s, 2H), 3.90 (d, J 1.2 Hz, 3H). ESI-MS m/z calc. 259.0667, found 260.1 (M+1)⁺; Retention time: 3.2 minutes; LC method T.

### Step 2: Methyl 6-chlorosulfonylpyridine-2-carboxylate

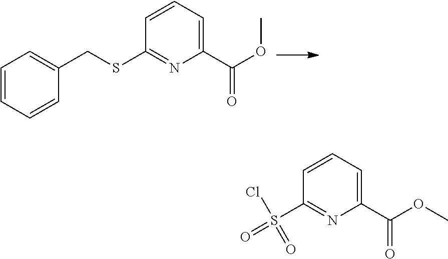 [SMILES: COC(=O)c1cccc(SCc2ccccc2)n1 ; COC(=O)c1cccc(S(=O)(=O)Cl)n1]

A solution of methyl 6-benzylsulfanylpyridine-2-carboxylate (121.62 g, 431.47 mmol) in DCM (950 mL) and DI water (300 mL) was cooled in a −1-0° C. ice bath and, with vigorous stirring, sulfuryl chloride (228.14 g, 140 mL, 1.6396 mol) was added dropwise while the temperature was maintained below 5° C. After the addition, the organic phase was separated, washed with DI water (2×500 mL), dried over anhydrous sodium sulfate, filtered and concentrated under vacuum. The residue was dissolved in DCM (500 mL). hexanes (1000 mL) was added and the DCM was slowly evaporated off. The white precipitate was filtered by vacuum and the solids were washed with hexanes (2×500 mL). The filtered solids were collected. The residue solids in the filtrate were filtered and dissolved in DCM (500 mL). The DCM solution was transferred to a 1 L round-bottom flask and concentrated under vacuum. The residue was dissolved in DCM (200 mL). hexanes (600 mL) was added and the DCM was slowly evaporated off. The white precipitation was filtered by vacuum and the solids were washed with hexanes (2×500 mL) After drying, methyl 6-chlorosulfonylpyridine-2-carboxylate (56.898 g, 55%) was isolated. ¹H NMR (500 MHz, Chloroform-d) δ 8.48 (dd, J 7.8, 1.1 Hz, 1H), 8.31 (dd, J 7.9, 1.1 Hz, 1H), 8.25 (t, J 7.8 Hz, 1H), 4.08 (s, 3H). ESI-MS m/z calc. 234.97061, found 236.1 (M+1)⁺; Retention time: 1.74 minutes; LC method T.

### Step 3: Methyl 6-[[4-chloro-6-(2,6-dimethylphenyl)pyrimidin-2-yl]sulfamoyl]pyridine-2-carboxylate

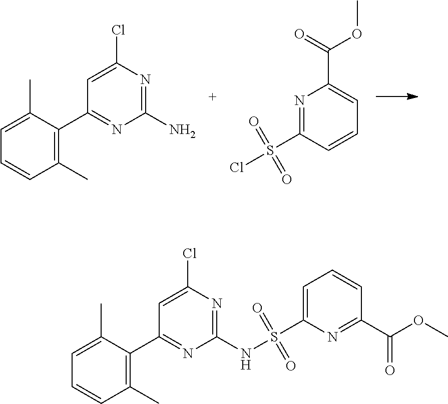 [SMILES: Cc1cccc(C)c1-c1cc(Cl)nc(N)n1 ; COC(=O)c1cccc(S(=O)(=O)Cl)n1 ; COC(=O)c1cccc(S(=O)(=O)Nc2nc(Cl)cc(-c3c(C)cccc3C)n2)n1]

A solution of 4-chloro-6-(2,6-dimethylphenyl)pyrimidin-2-amine (16.63 g, 71.161 mmol) and methyl 6-chlorosulfonylpyridine-2-carboxylate (16.8 g, 71.294 mmol) dissolved in anhydrous THE (680 mL) was cooled to −78° C. Then Lithium bis(trimethylsilyl)amide (143 mL of 1 M, 143.00 mmol) in solution in THE was added dropwise. The mixture was allowed to warm up to 0° C. slowly and then 1M aqueous HCl (146 mL) was added, followed by DI water (680 mL). The THF was evaporated and the aqueous phase was extracted with chloroform (3×250 mL). The combined organic layers were washed with saturated aqueous NaCl (300 mL), dried over anhydrous sodium sulfate, filtered, and concentrated under vacuum. The crude was recrystallized in 10% Acetone in hexanes (500 mL). The white precipitate was filtered and rinsed with acetone (2×100 mL) to give methyl 6-[[4-chloro-6-(2,6-dimethylphenyl)pyrimidin-2-yl]sulfamoyl]pyridine-2-carboxylate (15.79 g, 50%). ESI-MS m/z calc. 432.06592, found 433.3 (M+1)⁺; Retention time: 5.5 minutes; LC method S.

### Step 4: 6-[[4-Chloro-6-(2,6-dimethylphenyl)pyrimidin-2-yl]sulfamoyl]pyridine-2-carboxylic acid

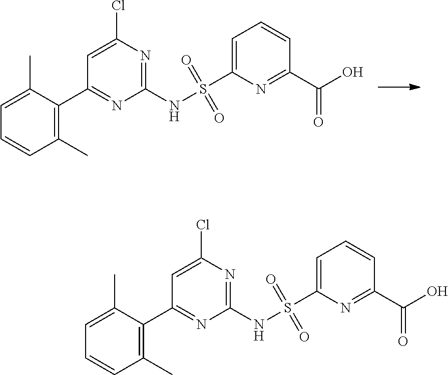 [SMILES: Cc1cccc(C)c1-c1cc(Cl)nc(NS(=O)(=O)c2cccc(C(=O)O)n2)n1 ; Cc1cccc(C)c1-c1cc(Cl)nc(NS(=O)(=O)c2cccc(C(=O)O)n2)n1]

To a solution of methyl 6-[[4-chloro-6-(2,6-dimethylphenyl)pyrimidin-2-yl]sulfamoyl]pyridine-2-carboxylate (15.79 g, 36.477 mmol) in THE (180 mL) was added aqueous sodium hydroxide (182 mL of 1 M, 182.00 mmol). The reaction was stirred at RT for 1h. The THF was evaporated, and the aqueous layer was washed with diethyl ether (2×200 mL). The aqueous layer was acidified to pH 2 with 1 M Aqueous HCl (250 mL). The precipitate was filtered and the a white solid were rinsed with DI water (2×250 mL). The solids were dried under vacuum to give 6-[[4-chloro-6-(2,6-dimethylphenyl)pyrimidin-2-yl]sulfamoyl]pyridine-2-carboxylic acid (14.3444 g, 93%). ¹H NMR (250 MHz, DMSO-d₆) δ 8.14-7.99 (m, 3H), 7.21-7.11 (m, 1H), 7.03 (d, J 7.7 Hz, 2H), 6.92 (s, 1H), 1.78 (s, 6H). ESI-MS m/z calc. 418.05026, found 419.1 (M+1)⁺; Retention time: 2.61 minutes; LC method T.

### Example J: Preparation of 3-({4-[(2R)-2-{[(tert-Butoxy)carbonyl]amino}-3-methylbutoxy]-6-(2,6-dimethylphenyl)pyrimidin-2-yl}sulfamoyl)benzoic acid

### Step 1: 3-({4-[(2R)-2-{[(tert-Butoxy)carbonyl]amino}-3-methylbutoxy]-6-(2,6-dimethylphenyl)pyrimidin-2-yl}sulfamoyl)benzoic acid

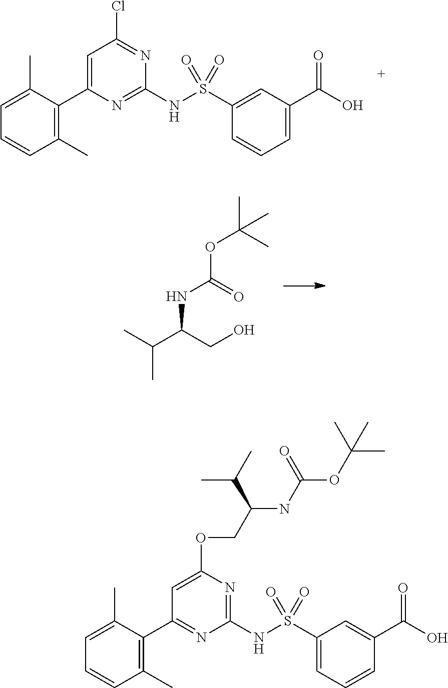

A solution of 3-[[4-chloro-6-(2,6-dimethylphenyl)pyrimidin-2-yl]sulfamoyl]benzoic acid (70 mg, 0.1675 mmol) in THE (653.3 μL) was added to tert-butyl N-[(1R)-1-(hydroxymethyl)-2-methyl-propyl]carbamate (approximately 51.08 mg, 0.2513 mmol). Solid sodium tert-butoxide (approximately 80.49 mg, 0.8375 mmol) was added last. The reaction mixture was allowed to stir overnight at room temperature. The reaction mixture was neutralized with the addition of aqueous HCl. The remaining suspension was diluted with DMSO (200 μL), filtered and purified by reverse phase HPLC using a Luna C₁₈ (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. 3-({4-[(2R)-2-{[(tert-butoxy)carbonyl]amino}-3-methylbutoxy]-6-(2,6-dimethylphenyl)pyrimidin-2-yl}sulfamoyl)benzoic acid was obtained (97 mg). ESI-MS m/z calc. 584.23047, found 585.2 (M+1)⁺; Retention time: 1.73 minutes; LC method A.

### Example K: Preparation of 3-[[4-[(2R)-2-Amino-3-cyclopropyl-propoxy]-6-(2,6-dimethylphenyl)pyrimidin-2-yl]sulfamoyl]benzoic acid

### Step 1: tert-Butyl N-[(1R)-1-(cyclopropylmethyl)-2-hydroxy-ethyl]carbamate

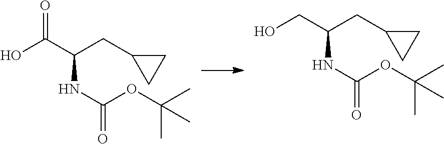 [SMILES: CC(C)(C)OC(=O)N[C@H](CC1CC1)C(=O)O ; CC(C)(C)OC(=O)N[C@@H](CO)CC1CC1]

A solution of (2R)-2-(tert-butoxycarbonylamino)-3-cyclopropyl-propanoic acid (0.22 g, 0.9596 mmol) and Borane-Tetrahydrofuran Complex (2.9 mL of 1 M, 2.900 mmol) in THE (5 mL) was stirred for three hours. The reaction was quenched with 1 M citric acid and extracted with ethyl acetate. The combined extracts were washed with water, dried over sodium sulfate, and evaporated under vacuum to give tert-butyl N-[(1R)-1-(cyclopropylmethyl)-2-hydroxy-ethyl]carbamate (89 mg, 43%). ESI-MS m/z calc. 215.15215, found 216.2 (M+1)⁺; Retention time: 0.47 minutes; LC method D.

### Step 2: 3-[[4-[(2R)-2-(tert-Butoxycarbonylamino)-3-cyclopropyl-propoxy]-6-(2,6-dimethylphenyl)pyrimidin-2-yl]sulfamoyl]benzoic acid

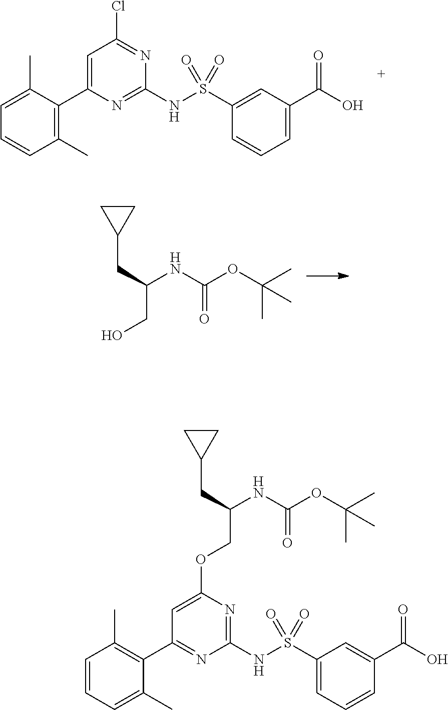 [SMILES: Cc1cccc(C)c1-c1cc(Cl)nc(NS(=O)(=O)c2cccc(C(=O)O)c2)n1 ; CC(C)(C)OC(=O)N[C@@H](CO)CC1CC1 ; Cc1cccc(C)c1-c1cc(OC[C@@H](CC2CC2)NC(=O)OC(C)(C)C)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (approximately 172.7 mg, 0.4134 mmol), tert-Butyl N-[(1R)-1-(cyclopropylmethyl)-2-hydroxy-ethyl]carbamate (89 mg, 0.4134 mmol), and sodium t-butoxide (approximately 159.0 mg, 1.654 mmol) in THE (2.067 mL) was stirred for 22 hours. The reaction was quenched with 1 M citric acid, diluted with water, and extracted with ethyl acetate. The combined extracts were washed with brine, dried over sodium sulfate, and evaporated. The residue was purified by silica gel column chromatography with 0-10% methanol in dichloromethane to give partially clean product. The impure product was re-purified using a reverse phase HPLC-MS method using a Luna C₁₈(2) column (75×30 mm, 5 μm particle size) sold by Phenomenex (pn: 00C-4252-U0-AX), and a dual gradient run from 1-99% mobile phase B over 15.0 minutes. Mobile phase A=H₂O (5 mM HCl). Mobile phase B=CH₃CN. Flow rate=50 mL/min, and column temperature=25° C. to give 3-[[4-[(2R)-2-(tert-butoxycarbonylamino)-3-cyclopropyl-propoxy]-6-(2,6-dimethylphenyl)pyrimidin-2-yl]sulfamoyl]benzoic acid (45 mg, 18%) obtained as a colorless solid. ESI-MS m/z calc. 596.23047, found 597.3 (M+1)⁺; Retention time: 0.68 minutes; LC method D.

### Step 3: 3-[[4-[(2R)-2-Amino-3-cyclopropyl-propoxy]-6-(2,6-dimethylphenyl)pyrimidin-2-yl]sulfamoyl]benzoic acid

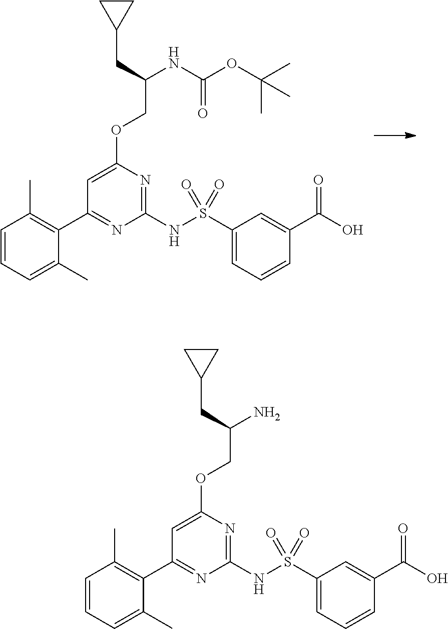 [SMILES: Cc1cccc(C)c1-c1cc(OC[C@@H](CC2CC2)NC(=O)OC(C)(C)C)nc(NS(=O)(=O)c2cccc(C(=O)O)c2)n1 ; Cc1cccc(C)c1-c1cc(OC[C@H](N)CC2CC2)nc(NS(=O)(=O)c2cccc(C(=O)O)c2)n1]

A solution of 3-[[4-[(2R)-2-(tert-butoxycarbonylamino)-3-cyclopropyl-propoxy]-6-(2,6-dimethylphenyl)pyrimidin-2-yl]sulfamoyl]benzoic acid (45 mg, 0.07542 mmol) in HCl (3 mL of 4 M, 12.00 mmol) (in dioxane) was stirred for four hours. The solvent was removed under vacuum, and the resulting solids were triturated with diethyl ether and dried under vacuum to give 3-[[4-[(2R)-2-amino-3-cyclopropyl-propoxy]-6-(2,6-dimethylphenyl)pyrimidin-2-yl]sulfamoyl]benzoic acid (hydrochloride salt) (53 mg, 132%). ESI-MS m/z calc. 496.17804, found 497.3 (M+1)⁺; Retention time: 0.41 minutes; LC method D.

### Example L: Preparation of 3-[[4-[(2R)-2-Amino-4-methyl-pentoxy]-6-(2,6-dimethylphenyl)-2-pyridyl]sulfamoyl]benzoic acid

### Step 1: 4-Chloro-6-(2,6-dimethylphenyl)pyridin-2-amine

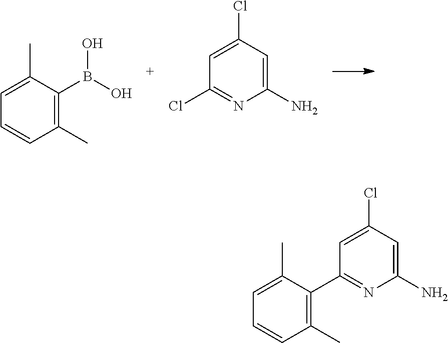 [SMILES: Cc1cccc(C)c1B(O)O ; Nc1cc(Cl)cc(Cl)n1 ; Cc1cccc(C)c1-c1cc(Cl)cc(N)n1]

To a stirring solution of (2,6-dimethylphenyl)boronic acid (11.515 g, 76.775 mmol) and 4,6-dichloropyridin-2-amine (12.513 g, 76.765 mmol) in Toluene (425 mL) and EtOH (213 mL) was added an aqueous solution of Sodium carbonate (115 mL of 2 M, 230.00 mmol) and the reaction mixture was degassed with nitrogen gas for 45 min. Pd(dppf)Cl₂ (6.271 g, 7.6791 mmol) was then added with degassing continuing for an additional 15 min. Then the reaction vial was sealed, and the mixture heated to 100° C. and stirred at that temperature for 24 h. After this time, volatiles were removed under reduced pressure and the residue was extracted with ethyl acetate (3×200 mL). The combined organic layers were washed with brine, dried over anhydrous sodium sulfate and concentrated under reduced pressure. The crude product was purified by silica gel column chromatography (0-25% EtOAc in hexanes) and triturated with hexanes to afford 4-chloro-6-(2,6-dimethylphenyl)pyridin-2-amine (6.469 g, 34%) as an off-white solid. ESI-MS m/z calc. 232.07672, found 233.1 (M+1)⁺; Retention time: 2.31 minutes; (LC method T).

### Step 2: Methyl 3-[[4-chloro-6-(2,6-dimethylphenyl)-2-pyridyl]sulfamoyl]benzoate

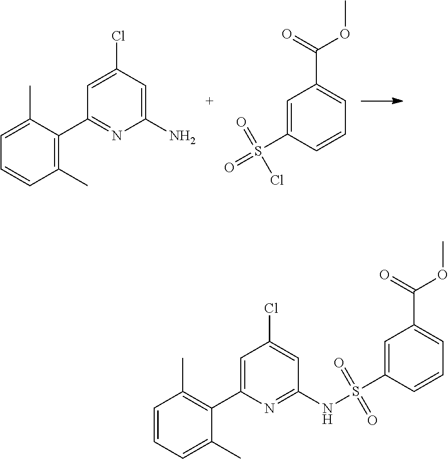 [SMILES: Cc1cccc(C)c1-c1cc(Cl)cc(N)n1 ; COC(=O)c1cccc(S(=O)(=O)Cl)c1 ; COC(=O)c1cccc(S(=O)(=O)Nc2cc(Cl)cc(-c3c(C)cccc3C)n2)c1]

To a solution of 4-chloro-6-(2,6-dimethylphenyl)pyridin-2-amine (4.9 g, 20.635 mmol) and methyl 3-chlorosulfonylbenzoate (4.9 g, 20.046 mmol) in THE (200 mL) was added dropwise Lithium bis(trimethylsilyl)amide (45 mL of 1 M, 45.000 mmol) at −78° C. under nitrogen. The reaction mixture was stirred for 30 minutes at −78° C.; then warmed up to 0° C. and stirred for 2 hours at 0° C. The reaction was quenched with cold 1.0 M Hydrochloric acid (50 mL) and diluted with water (200 mL). The mixture was extracted with ethyl acetate (2×400 mL). The organic layers were combined, washed with brine (500 mL), dried over sodium sulfate, filtered and concentrated. The residue was purified by chromatography using 0-20% ethyl acetate in hexanes to afford methyl 3-[[4-chloro-6-(2,6-dimethylphenyl)-2-pyridyl]sulfamoyl]benzoate (6.2 g, 68%) as a white solid. ESI-MS m/z calc. 430.0754, found 431.5 (M+1)⁺; Retention time: 3.65 minutes; (LC method T).

### Step 3: 3-[[4-chloro-6-(2,6-dimethylphenyl)-2-pyridyl]sulfamoyl]benzoic acid

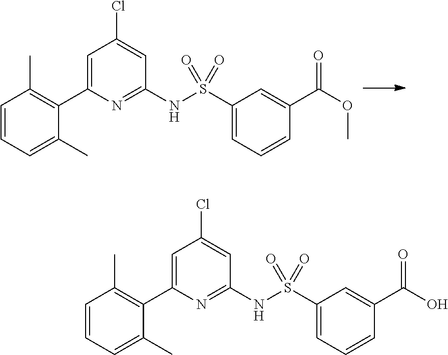 [SMILES: COC(=O)c1cccc(S(=O)(=O)Nc2cc(Cl)cc(-c3c(C)cccc3C)n2)c1 ; Cc1cccc(C)c1-c1cc(Cl)cc(NS(=O)(=O)c2cccc(C(=O)O)c2)n1]

To a stirring solution of 3-[4-chloro-6-(2,6-dimethyl-phenyl)-pyridin-2-ylsulfamoyl]-benzoic acid methyl ester (5.3 g, 12.3 mmol) in a mixture of tetrahydrofuran (80 mL) and water (80 mL) at room temperature was added lithium hydroxide monohydrate (1.55 g, 36.9 mmol) and the reaction mixture was stirred at 45° C. for 2 hours. Tetrahydrofuran was removed under vacuum and the residue was diluted with water (100 mL). The aqueous layer was washed with diethyl ether (2×50 mL), hexanes (50 mL) and acidified with 1.0 M hydrochloric acid to pH=2-3. The precipitated product was collected by filtration and dried in a vacuum oven at 75° C. to constant weight to afford 3-[4-chloro-6-(2,6-dimethyl-phenyl)-pyridin-2-ylsulfamoyl]-benzoic acid (4.8 g, 93%) as a white solid. H NMR (250 MHz, DMSO-d₆) δ (ppm): 8.32 (d, J 1.9 Hz, 1H), 8.14 (d, J 7.7 Hz, 1H), 8.03 (d, J 8.0 Hz, 1H), 7.63 (t, J 7.8 Hz, 1H), 7.28-6.96 (m, 5H), 1.77 (s, 6H). ESI-MS m/z calc. 416.8, found 417.0 (M1). Retention time: 5.11 minutes.

### Step 4: 3-[[4-[(2R)-2-Amino-4-methyl-pentoxy]-6-(2,6-dimethylphenyl)-2-pyridyl]sulfamoyl]benzoic acid

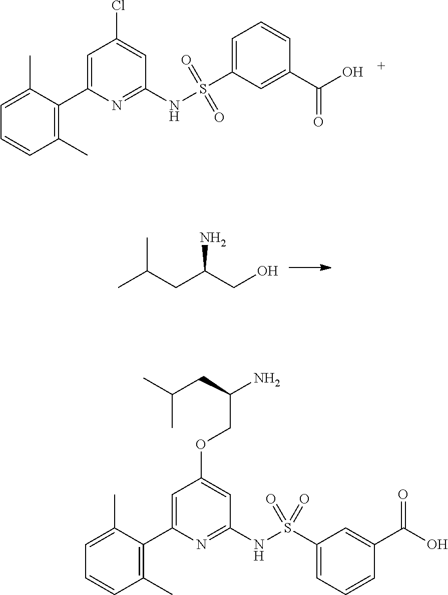

A 20 mL vial was charged with 3-[[4-chloro-6-(2,6-dimethylphenyl)-2-pyridyl]sulfamoyl]benzoic acid (300 mg, 0.7196 mmol), (2R)-2-amino-4-methyl-pentan-1-ol (110 mg, 0.9387 mmol) and anhydrous tetrahydrofuran (12 mL), in that order. Then the vial was purged with nitrogen for 30 seconds, and solid potassium tert-butoxide (350 mg, 3.119 mmol) was added capped under nitrogen. After stirred at 105° C. for 14 h (overnight), the reaction was allowed to cool to ambient temperature. Then glacial acetic acid (200 μL, 3.517 mmol) was added and the volatiles were removed under reduced pressure. To the residue, DMSO (5 mL) was added and microfiltered. Purification by reverse phase chromatography (Cis column, 1-99% acetonitrile in water over 15 min) gave 3-[[4-[(2R)-2-amino-4-methyl-pentoxy]-6-(2,6-dimethylphenyl)-2-pyridyl]sulfamoyl]benzoic acid (hydrochloride salt)(278 mg, 72%) as yellowish solid. ESI-MS m/z calc. 497.19846, found 498.2 (M+1)⁺; Retention time: 0.43 minutes (LC method D).

### Example M: Preparation of methyl 3-[[4-chloro-6-(2,6-dimethylphenyl)pyrimidin-2-yl]-(methoxymethyl)sulfamoyl]benzoate

### Step 1: Methyl 3-[[4-chloro-6-(2,6-dimethylphenyl)pyrimidin-2-yl]-(methoxymethyl)sulfamoyl]benzoate

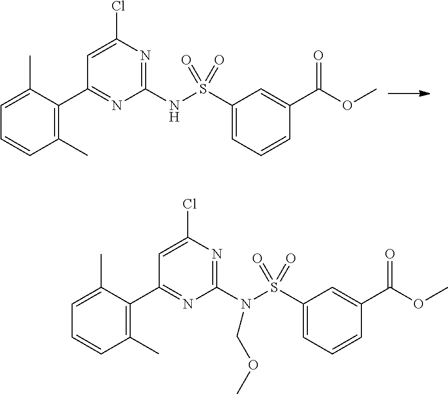 [SMILES: COC(=O)c1cccc(S(=O)(=O)Nc2nc(Cl)cc(-c3c(C)cccc3C)n2)c1 ; COCN(c1nc(Cl)cc(-c2c(C)cccc2C)n1)S(=O)(=O)c1cccc(C(=O)OC)c1]

To a solution of methyl 3-[[4-chloro-6-(2,6-dimethylphenyl)pyrimidin-2-yl]sulfamoyl]benzoate (35.04 g, 81.131 mmol) in Acetonitrile (525 mL) and 1,2-dichloroethane (525 mL) was added potassium carbonate (16.8 g, 121.56 mmol) followed by Chloromethyl methyl ether (7.5260 g, 7.1 mL, 93.475 mmol). The reaction mixture was stirred at room temperature for overnight. The solvent was evaporated, and the resulting material was partitioned between water (300 mL) and EtOAc (300 mL). The aqueous layer was extracted with EtOAc (2×200 mL). The combined organic layers were washed with water (300 mL) and brine (300 mL), dried over anhydrous sodium sulfate and concentrated under vacuum. The residue was purified by silica gel chromatography using 0 to 40% EtOAc in Hexane to afford methyl 3-[[4-chloro-6-(2,6-dimethylphenyl)pyrimidin-2-yl]-(methoxymethyl)sulfamoyl]benzoate (30.95 g, 80%) as clear jell. ESI-MS m/z calc. 475.0969, found 476.3 (M+1)⁺; Retention time: 3.96 minutes, LC method T.

### Example N: Preparation of 3-[[4-[2-amino-4-[1-(trifluoromethyl)cyclopropyl]butoxy]-6-(2,6-dimethylphenyl)pyrimidin-2-yl]sulfamoyl]benzoic acid

### Step 1: 3-[1-(Trifluoromethyl)cyclopropyl]propanal

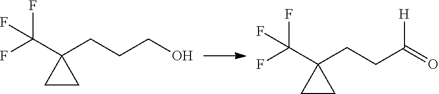 [SMILES: OCCCC1(C(F)(F)F)CC1 ; O=CCCC1(C(F)(F)F)CC1]

Dess-Martin periodinane (880 mg, 2.075 mmol) was added to a stirred solution of 3-[1-(trifluoromethyl)cyclopropyl]propan-1-ol (350 mg, 1.665 mmol) in anhydrous methylene chloride (10 mL) at 0° C. (ice-water bath) under nitrogen. After 15 min, the bath was removed, and the reaction was allowed to warm to ambient temperature and stirring continued for another 3 h. The reaction was diluted with ether (60 mL) and saturated aqueous sodium bicarbonate (20 mL) was added slowly (to mitigate CO₂ gas evolution). Then sodium thiosulfate (10 mL) was added and stirred at ambient temperature for 30 min. The layers were separated, and the aqueous layer was extracted with ether (2×20 mL). The combined organics were washed with brine (20 mL), dried over anhydrous sodium sulfate, filtered and concentrated under reduced pressure (pressure set at 300 mbar) to afford 3-[1-(trifluoromethyl)cyclopropyl]propanal (250 mg, 90%) as a yellow oil. ¹H NMR (400 MHz, Benzene-d₆) δ 9.15 (s, 1H), 1.99-1.90 (m, 2H), 1.52-1.44 (m, 2H), 0.68-0.59 (m, 2H), 0.00 (dd, J=2.5, 1.6 Hz, 2H).

### Step 2: 2-(Benzylamino)-4-[1-(trifluoromethyl)cyclopropyl]butanenitrile

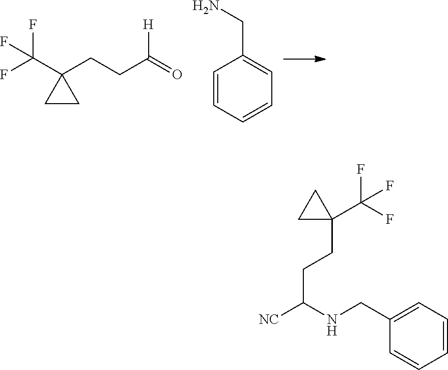

To a stirring solution of 3-[1-(trifluoromethyl)cyclopropyl]propanal (854 mg, 5.140 mmol) in acetonitrile (50.09 mL) under nitrogen atmosphere was added benzylamine (561.5 μL, 5.141 mmol) and trimethylsilylformonitrile (822.4 μL, 6.168 mmol). bromo(dimethyl)sulfonium bromide (114.1 mg, 0.5141 mmol) was then added and the mixture was stirred 2h. Removed 90% of the acetonitrile by rotary evaporation then added water (50.09 mL). Extracted the resulting mixture with EtOAc (3×), combined org. phases, dried (sodium sulfate), filtered and conc. to light tan oil which became a light tan solid on the high vacuum pump, 2-(benzylamino)-4-[1-(trifluoromethyl)cyclopropyl]butanenitrile (1.33 g, 92%) ESI-MS m/z calc. 282.13437, found 283.0 (M+1)⁺; Retention time: 0.56 minutes, LC method D.

### Step 3: 2-(Benzylamino)-4-[1-(trifluoromethyl)cyclopropyl]butanoic acid

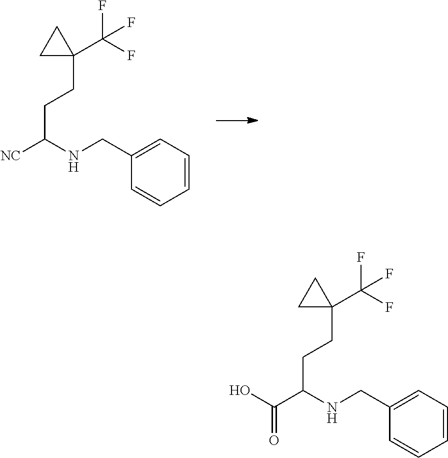 [SMILES: N#CC(CCC1(C(F)(F)F)CC1)NCc1ccccc1 ; O=C(O)C(CCC1(C(F)(F)F)CC1)NCc1ccccc1]

To a stirring solution of 2-(benzylamino)-4-[1-(trifluoromethyl)cyclopropyl]butanenitrile (1.33 g, 4.711 mmol) in acetic acid (897.3 μL, 15.78 mmol) in a vial was added HCl (8.96 mL of 37% w/v, 90.92 mmol) and the vial was capped. The mixture was stirred and heated in an aluminum block at 95° C. for 2 d. The mixture was transferred to a round bottom flask using MeOH and was concentrated by rotary evaporation, including treatment with diethyl ether and removing the solvents three times to give 2-(benzylamino)-4-[1-(trifluoromethyl)cyclopropyl]butanoic acid as a light tan solid that was dried thoroughly on the high vacuum pump then taken directly to the next step. (1.432 g, 100%) ESI-MS m/z calc. 301.12897, found 302.1 (M+1)⁺; Retention time: 0.37 minutes, LC method D.

### Step 4: 2-(Benzylamino)-4-[1-(trifluoromethyl)cyclopropyl]butan-1-ol

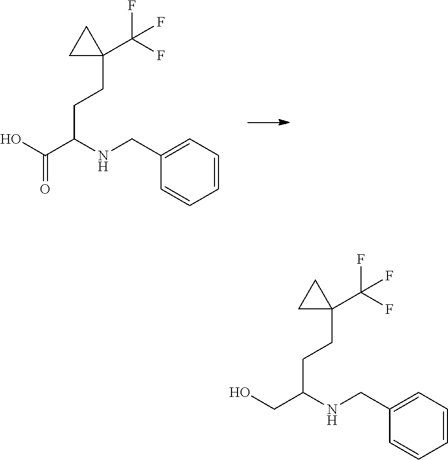 [SMILES: O=C(O)C(CCC1(C(F)(F)F)CC1)NCc1ccccc1 ; OCC(CCC1(C(F)(F)F)CC1)NCc1ccccc1]

To a stirring solution of 2-(benzylamino)-4-[1-(trifluoromethyl)cyclopropyl]butanoic acid (1.432 g, 4.705 mmol) in THE (28.36 mL) under nitrogen atmosphere at 0° C. was slowly added LAH (733.3 mg, 18.82 mmol) and the resulting mixture was stirred at 0° C. for 2 min then allowed to warm to rt and was stirred 75 min. Cooled to 0° C. and quenched by the addition of water (1.410 mL, 78.27 mmol), then KOH (1.411 mL of 15% w/v, 3.772 mmol) then water (2.819 mL, 156.5 mmol). Warmed to rt, added Celite and stirred 5 min then filtered over Celite eluting with ether. The ethereal filtrate was then dried (magnesium sulfate), filtered and concentrated the filtrate by rotary evaporation to give 2-(benzylamino)-4-[1-(trifluoromethyl)cyclopropyl]butan-1-ol (1.5146 g, 100%) as an orange oil which was used directly in the next step.ESI-MS m/z calc. 287.1497, found 288.0 (M+1)⁺; Retention time: 0.39 minutes, LC method D.

### Step 5: 3-[[4-[2-(Benzylamino)-4-[1-(trifluoromethyl)cyclopropyl]butoxy]-6-(2,6-dimethylphenyl)pyrimidin-2-yl]sulfamoyl]benzoic acid

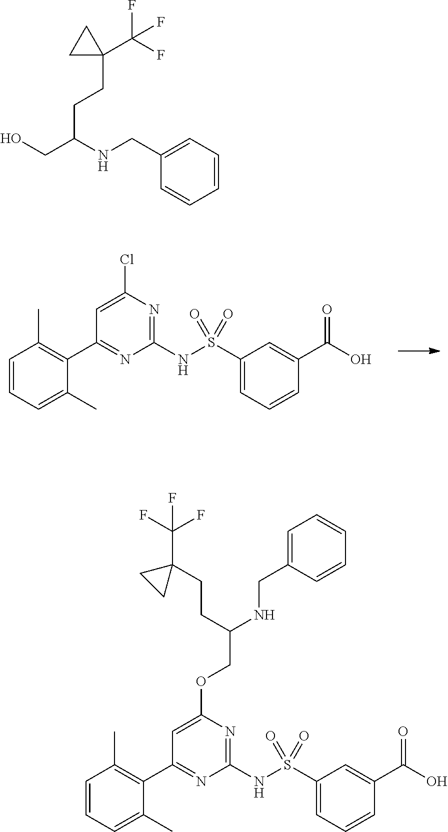 [SMILES: OCC(CCC1(C(F)(F)F)CC1)NCc1ccccc1 ; Cc1cccc(C)c1-c1cc(Cl)nc(NS(=O)(=O)c2cccc(C(=O)O)c2)n1 ; Cc1cccc(C)c1-c1cc(OCC(CCC2(C(F)(F)F)CC2)NCc2ccccc2)nc(NS(=O)(=O)c2cccc(C(=O)O)c2)n1]

To a stirring solution of 3-[[4-chloro-6-(2,6-dimethylphenyl)pyrimidin-2-yl]sulfamoyl]benzoic acid (647.3 mg, 1.549 mmol) and 2-(benzylamino)-4-[1-(trifluoromethyl)cyclopropyl]butan-1-ol (500 mg, 1.549 mmol) in THE (9.79 mL) at 0° C. was added KOtBu (770.8 μL, 6.196 mmol) and the mixture was stirred at 50° C. for 20 min then removed the THF by rotary evaporation, dissolved the residue in DMSO, filtered and chromatographed on a 275 g Reverse Phase Column eluting with 20-100% ACN/Water giving 3-[[4-[2-(benzylamino)-4-[1-(trifluoromethyl)cyclopropyl]butoxy]-6-(2,6-dimethylphenyl)pyrimidin-2-yl]sulfamoyl]benzoic acid (670 mg, 65%). ESI-MS m/z calc. 668.228, found 669.1 (M+1)⁺; Retention time: 0.54 minutes, LC method D.

### Step 6: 3-[[4-[2-amino-4-[1-(trifluoromethyl)cyclopropyl]butoxy]-6-(2,6-dimethylphenyl)pyrimidin-2-yl]sulfamoyl]benzoic acid

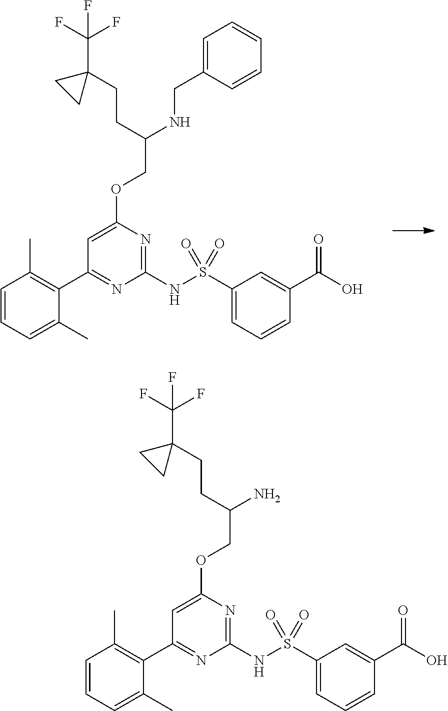 [SMILES: Cc1cccc(C)c1-c1cc(OCC(CCC2(C(F)(F)F)CC2)NCc2ccccc2)nc(NS(=O)(=O)c2cccc(C(=O)O)c2)n1 ; Cc1cccc(C)c1-c1cc(OCC(N)CCC2(C(F)(F)F)CC2)nc(NS(=O)(=O)c2cccc(C(=O)O)c2)n1]

A mixture of 3-[[4-[2-(benzylamino)-4-[1-(trifluoromethyl)cyclopropyl]butoxy]-6-(2,6-dimethylphenyl)pyrimidin-2-yl]sulfamoyl]benzoic acid (hydrochloride salt) (359 mg, 0.5368 mmol) and palladium(2+);dihydroxide (37.69 mg of 20% w/w, 0.05368 mmol) in Ethanol (8.0 mL) and HCl (1.1 mL of 1 M, 1.100 mmol) was purged with hydrogen gas (1 mg, 0.4961 mmol) and vigorously stirred under a hydrogen atmosphere for 6 hours. The reaction was filtered and concentrated under vacuum to give 3-[[4-[2-amino-4-[1-(trifluoromethyl)cyclopropyl]butoxy]-6-(2,6-dimethylphenyl)pyrimidin-2-yl]sulfamoyl]benzoic acid (hydrochloride salt) (303 mg, 92%) ESI-MS m/z calc. 578.1811, found 579.1 (M+1)⁺; Retention time: 1.14 minutes as a solid, LC method A.

### Example O: Preparation of 3-[[4-(2-amino-4,4,4-trifluoro-butoxy)-6-(2,6-dimethylphenyl)pyrimidin-2-yl]sulfamoyl]benzoic acid

### Step 1: 3-[[4-[2-(tert-Butoxycarbonylamino)-4,4,4-trifluoro-butoxy]-6-(2,6-dimethylphenyl)pyrimidin-2-yl]sulfamoyl]benzoic acid

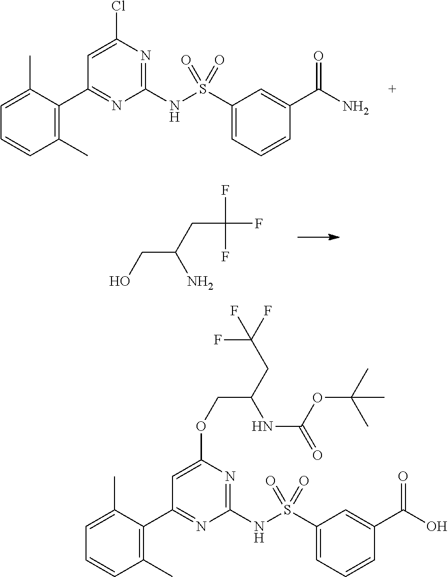 [SMILES: Cc1cccc(C)c1-c1cc(Cl)nc(NS(=O)(=O)c2cccc(C(N)=O)c2)n1 ; NC(CO)CC(F)(F)F ; Cc1cccc(C)c1-c1cc(OCC(CC(F)(F)F)NC(=O)OC(C)(C)C)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (0.63 g, 1.508 mmol), 2-amino-4,4,4-trifluoro-butan-1-ol (hydrochloride salt) (0.54 g, 3.007 mmol), and sodium t-butoxide (0.73 g, 7.596 mmol) in THF (8 mL) was stirred for five minutes, turning bright yellow. The reaction was placed in a preheated 60° C. bath and stirred for 25 minutes. UPLCMS showed complete conversion to amino intermediate. After cooling to room temperature, di-tert-butyl dicarbonate (0.67 g, 3.070 mmol) was added, and the reaction was stirred for 17 hours. The reaction was quenched with 1 M hydrochloric acid, diluted with water, and extracted with ethyl acetate. The combined extracts were washed with water, dried over sodium sulfate, and evaporated under vacuum. The residue was purified by silica gel column chromatography with 0-10% methanol in dichloromethane to give a mixture containing product. The mixture was re-purified by silica gel column chromatography with 0-9% methanol in dichloromethane to give 3-[[4-[2-(tert-butoxycarbonylamino)-4,4,4-trifluoro-butoxy]-6-(2,6-dimethylphenyl)pyrimidin-2-yl]sulfamoyl]benzoic acid (0.54 g, 57%) ESI-MS m/z calc. 624.1866, found 625.3 (M+1)⁺; Retention time: 0.67 minutes as a colorless solid, LC method D.

### Step 2: 3-[[4-(2-Amino-4,4,4-trifluoro-butoxy)-6-(2,6-dimethylphenyl)pyrimidin-2-yl]sulfamoyl]benzoic acid

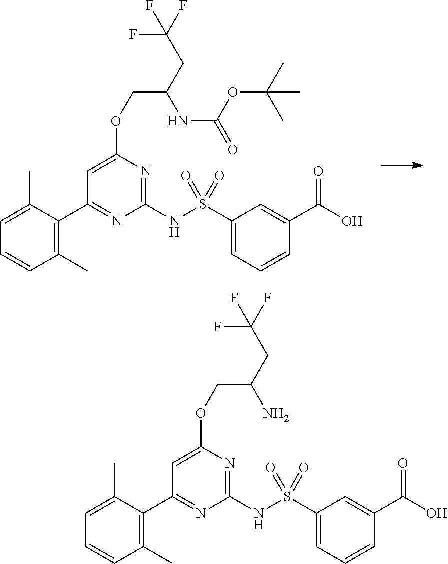

A solution of 3-[[4-[2-(tert-butoxycarbonylamino)-4,4,4-trifluoro-butoxy]-6-(2,6-dimethylphenyl)pyrimidin-2-yl]sulfamoyl]benzoic acid (83 mg, 0.1329 mmol) and HCl (4 mL of 4 M, 16.00 mmol) (in dioxane) was stirred for one hour. The solvent was removed under vacuum, and the solids were triturated with diethyl ether to give 3-[[4-(2-amino-4,4,4-trifluoro-butoxy)-6-(2,6-dimethylphenyl)pyrimidin-2-yl]sulfamoyl]benzoic acid (hydrochloride salt) (81 mg, 109%) ESI-MS m/z calc. 524.13416, found 525.2 (M+1)⁺; Retention time: 0.39 minutes as a colorless solid, LC method D.

### Example P: Preparation of 3-[[4-[(2R)-2-aminopropoxy]-6-(2,6-dimethylphenyl)pyrimidin-2-yl]sulfamoyl]benzoic acid

### Step 1: 3-[[4-[(2R)-2-(tert-Butoxycarbonylamino)propoxy]-6-(2,6-dimethylphenyl)pyrimidin-2-yl]sulfamoyl]benzoic acid

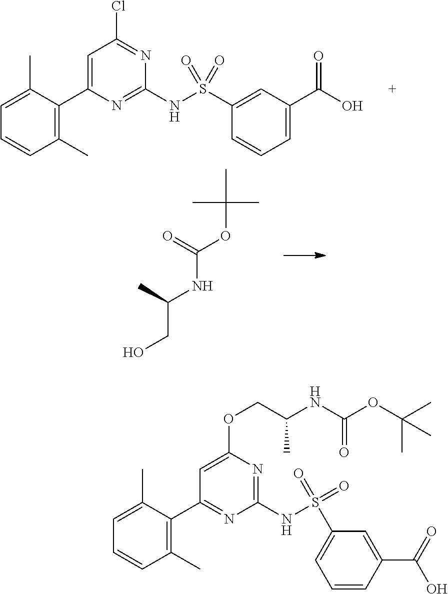

A solution of 3-[[4-chloro-6-(2,6-dimethylphenyl)pyrimidin-2-yl]sulfamoyl]benzoic acid (75 mg, 0.1795 mmol) in THE (0.7 mL) was added to tert-butyl N-[(1R)-2-hydroxy-1-methyl-ethyl]carbamate (approximately 47.17 mg, 0.2692 mmol). Solid sodium tert-butoxide (approximately 86.25 mg, 0.8975 mmol) was added after. The reaction mixture was allowed to stir overnight at room temperature. acetic acid (approximately 64.68 mg, 61.25 μL, 1.077 mmol) was added. The reaction mixture was diluted with DCM and washed with HCl (1 M, 1×7 mL) and brine (2×75 mL). The organic layer was dried over sodium sulfate, filtered and concentrated under reduced pressure. The crude product was chromatographed on a 12 gram silica gel column eluting with a EtOAc/hexane gradient. 3-[[4-[(2R)-2-(tert-Butoxycarbonylamino)propoxy]-6-(2,6-dimethylphenyl)pyrimidin-2-yl]sulfamoyl]benzoic acid (1.65 g, 2.964 mmol) (65 mg, 65%) was obtained. ESI-MS m/z calc. 556.19916, found 557.3 (M+1)⁺; Retention time: 1.63 minutes; LC method A.

### Step 2: 3-[[4-[(2R)-2-Aminopropoxy]-6-(2,6-dimethylphenyl)pyrimidin-2-yl]sulfamoyl]benzoic acid

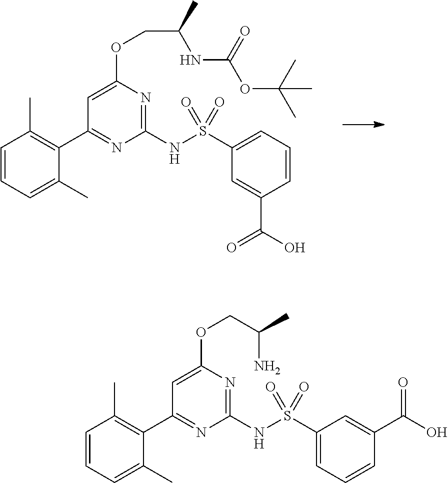 [SMILES: Cc1cccc(C)c1-c1cc(OC[C@@H](C)NC(=O)OC(C)(C)C)nc(NS(=O)(=O)c2cccc(C(=O)O)c2)n1 ; Cc1cccc(C)c1-c1cc(OC[C@@H](C)N)nc(NS(=O)(=O)c2cccc(C(=O)O)c2)n1]

A solution of 3-[[4-[(2R)-2-(tert-butoxycarbonylamino)propoxy]-6-(2,6-dimethylphenyl)pyrimidin-2-yl]sulfamoyl]benzoic acid (1.65 g, 2.964 mmol) in HCl (8 mL of 4 M, 32.00 mmol) (in dioxane) was stirred for two hours, and the solvent was removed under vacuum. The solids were triturated with diethyl ether and dried under vacuum to give 3-[[4-[(2R)-2-aminopropoxy]-6-(2,6-dimethylphenyl)pyrimidin-2-yl]sulfamoyl]benzoic acid (hydrochloride salt) (1.55 g, 106%) as a colorless solid. ESI-MS m/z calc. 456.14673, found 457.2 (M+1)⁺; Retention time: 0.37 minutes, LC method D.

### Example Q: Preparation of 5-[[4-[(2R)-2-amino-4-methyl-pentoxy]-6-(2,6-dimethylphenyl)pyrimidin-2-yl]sulfamoyl]-2-methyl-pyrazole-3-carboxylic acid

### Step 1: Ethyl 3-nitro-1H-pyrazole-5-carboxylate

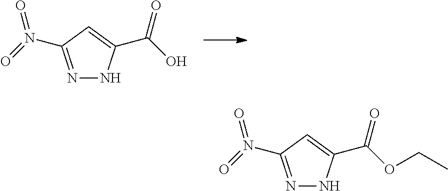

To a solution 3-nitro-1H-pyrazole-5-carboxylic acid (25 g, 159.15 mmol) in EtOH (250 mL) at rt was added acetyl chloride (37.536 g, 34 mL, 478.18 mmol) slowly. The mixture was stirred at reflux for 4 h. The mixture was concentrated and co-evaporated with EtOH (100 mL) and 1,4-dioxane (50 mL) to give ethyl 3-nitro-1H-pyrazole-5-carboxylate (30 g, 1000) as off-white solid. ESI-MS m/z calc. 185.0437, found 186.1 (M+1)⁺; Retention time: 1.58 minutes. ¹H NMR (300 MHz, CDCl₃) δ 7.41 (s, 1H), 4.47 (q, J=7.0 Hz, 2H), 1.43 (t, J=7.0 Hz, 3H), 1.25 (s, 1H), LC method K.

### Step 2: Ethyl 2-methyl-5-nitro-pyrazole-3-carboxylate

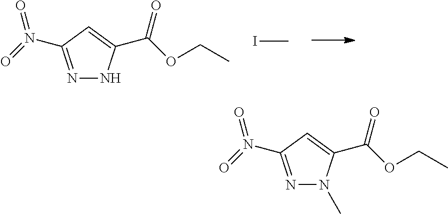

To a solution of ethyl 3-nitro-1H-pyrazole-5-carboxylate (29.6 g, 154.61 mmol) in DMF (200 mL) at 0° C. was added potassium carbonate (44.2 g, 319.81 mmol) and iodomethane (34.200 g, 15 mL, 240.95 mmol) dropwise over 15 min. The mixture was stirred at rt overnight. The mixture was cooled with ice-water bath and cold water (600 mL) was added. The precipitate was collected by filtration and washed with cold water. The resulting precipitate was dissolved in EtOAc (200 mL), dried over sodium sulfate, filtered and concentrated to dryness to give Flash ethyl 2-methyl-5-nitro-pyrazole-3-carboxylate (24.55 g, 78%) as a pale orange solid. ¹H NMR (400 MHz, CDCl₃) δ 7.41 (s, 1H), 4.42 (q, J 7.3 Hz, 2H), 4.29 (s, 3H), 1.42 (t, J 7.2 Hz, 3H). ESI-MS m/z calc. 199.0593, found 200.2 (M+1)⁺; Retention time: 1.66 minutes (LC method X).

### Step 3: Ethyl 5-amino-2-methyl-pyrazole-3-carboxylate

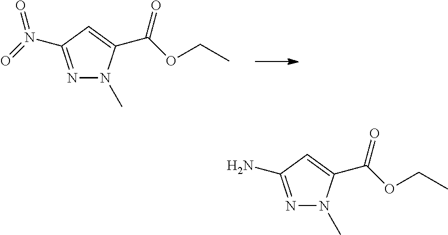

A mixture of ethyl 2-methyl-5-nitro-pyrazole-3-carboxylate (24.74 g, 124.22 mmol), 10% Palladium on carbon 50% wet (8 g, 3.7587 mmol) and MeOH (250 mL) was hydrogenated under hydrogen (balloon) for 24 h. The mixture was filtered through diatomaceous earth and washed with EtOAc. The filtrate was concentrated to give ethyl 5-amino-2-methyl-pyrazole-3-carboxylate (20.88 g, 99%) as white solid. ESI-MS m/z calc. 169.0851, found 170.1 (M+1)⁺; Retention time: 1.33 minutes. ¹H NMR (300 MHz, CDCl₃) δ 6.13 (s, 1H), 4.30 (q, J=7.1 Hz, 2H), 3.99 (s, 3H), 3.62 (br. s., 2H), 1.35 (t, J=7.0 Hz, 3H). LC method K.

### Step 4: Ethyl 5-chlorosulfonyl-2-methyl-pyrazole-3-carboxylate

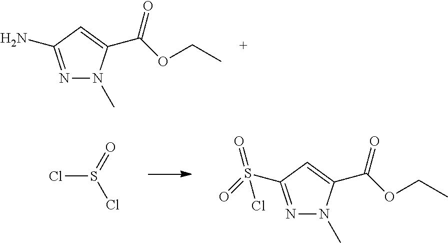

A 500-mL three-neck flask was charged with water (200 mL) and cooled with ice-water bath. Thionyl chloride (66.055 g, 40.5 mL, 555.22 mmol) was added dropwise over 20 minutes. The mixture was stirred at room temperature for 2 hours. Copper(I) chloride (800 mg, 8.0809 mmol) was added and the mixture was cooled to −5° C. Another 250-mL flask was charged with hydrochloric acid solution (37 wt %) (120 mL of 12 M, 1.4400 mol) and ethyl 5-amino-2-methyl-pyrazole-3-carboxylate (20.23 g, 107.38 mmol) was added. The mixture was cooled to −5° C. and a solution of sodium nitrite (9.26 g, 134.21 mmol) in water (50 mL) was added dropwise over 30 minutes, keeping the inner temperature between −6° C. and −3° C. The mixture was stirred at ˜5° C. for 30 minutes, cooled to −10° C., and slowly canulated (˜25 minutes) to the first solution. The resulting mixture was stirred at 0-5° C. (ice-water bath) for 90 minutes. More copper(I) chloride (270 mg, 2.7273 mmol) was added and the resulting mixture was stirred at 0-5° C. (ice-water bath) for 1 hour. The mixture was extracted with ethyl acetate (2×200 mL), the organic layer was dried with sodium sulfate, filtered and concentrated to dryness. The crude material was purified in two equal batches by Flash chromatography on silica gel (120 g silica gel+100 g) eluted with 0% to 20% ethyl acetate in heptane to afford ethyl 5-chlorosulfonyl-2-methyl-pyrazole-3-carboxylate (12.1 g, 43%) as a colorless oil. ¹H NMR (400 MHz, CDCl₃) δ 7.40 (s, 1H), 4.42 (q, J 7.1 Hz, 2H), 4.33 (s, 3H), 1.42 (t, J 7.1 Hz, 3H). ESI-MS m/z calc. 251.9972, found 253.0 (M+1)⁺; Retention time: 4.03 minutes (LC method Y).

### Step 5: Ethyl 5-[[4-chloro-6-(2,6-dimethylphenyl)pyrimidin-2-yl]sulfamoyl]-2-methyl-pyrazole-3-carboxylate

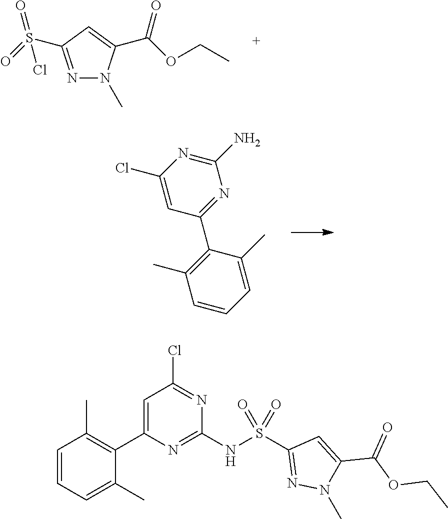 [SMILES: CCOC(=O)c1cc(S(=O)(=O)Cl)nn1C ; Cc1cccc(C)c1-c1cc(Cl)nc(N)n1 ; CCOC(=O)c1cc(S(=O)(=O)Nc2nc(Cl)cc(-c3c(C)cccc3C)n2)nn1C]

To a solution of 4-chloro-6-(2,6-dimethylphenyl)pyrimidin-2-amine (4.8 g, 20.539 mmol) in THE (140 mL) at 0° C. was added a solution of ethyl 5-chlorosulfonyl-2-methyl-pyrazole-3-carboxylate (6.13 g, 23.217 mmol), followed by sodium tert-amoxide in toluene (13.9 mL of 40% w/v, 50.486 mmol) dropwise. The mixture was stirred at rt for 1.5 h. The mixture was slowly poured into a 1 N aqueous HCl (50 mL) at 0° C. The mixture was diluted with water 100 mL and extracted with EtOAc (3×100 mL). The combined organic layers were dried over sodium sulfate filtered and concentrated to dryness. The crude material was purified by flash chromatography on silica gel (330 g) eluted with 5% to 30% ethyl acetate in heptane and the 100% ethyl acetate to give ethyl 5-[[4-chloro-6-(2,6-dimethylphenyl)pyrimidin-2-yl]sulfamoyl]-2-methyl-pyrazole-3-carboxylate (6.77 g, 72%) as white solid. ¹H NMR (400 MHz, CDCl₃) δ 7.95 (br. s., 1H), 7.49 (s, 1H), 7.23 (t, J 8.1 Hz, 1H), 7.09 (d, J 7.6 Hz, 2H), 6.94 (s, 1H), 4.36 (q, J 7.3 Hz, 2H), 4.24 (s, 3H), 2.03 (s, 6H), 1.37 (t, J 7.2 Hz, 3H). ESI-MS m/z calc. 449.0925, found 450.2 (M+1)⁺; Retention time: 4.42 minutes (LC method A).

### Step 6: 5-[[4-Chloro-6-(2,6-dimethylphenyl)pyrimidin-2-yl]sulfamoyl]-2-methyl-pyrazole-3-carboxylic acid

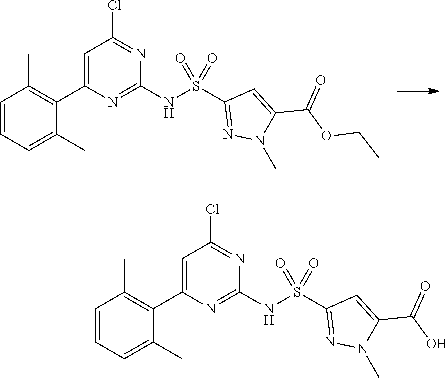 [SMILES: CCOC(=O)c1cc(S(=O)(=O)Nc2nc(Cl)cc(-c3c(C)cccc3C)n2)nn1C ; Cc1cccc(C)c1-c1cc(Cl)nc(NS(=O)(=O)c2cc(C(=O)O)n(C)n2)n1]

To a solution of ethyl 5-[[4-chloro-6-(2,6-dimethylphenyl)pyrimidin-2-yl]sulfamoyl]-2-methyl-pyrazole-3-carboxylate (7.62 g, 16.598 mmol) in THE (220 mL) at 0° C. was added a solution of NaOH (2.7 g, 67.505 mmol) in water (50 mL) and the mixture was stirred for 20 minutes. The mixture was concentrated to remove THF, diluted with water (100 mL) and washed with ethyl acetate (2×100 mL); the combined organic layers were discarded. The aqueous layer was cooled to 0° C., acidified to pH 3-4 with 1N aqueous HCl and extracted with ethyl acetate (3×150 mL). The combined organic layers were dried over sodium sulfate, filtered and concentrated to dryness to give 5-[[4-chloro-6-(2,6-dimethylphenyl)pyrimidin-2-yl]sulfamoyl]-2-methyl-pyrazole-3-carboxylic acid (7.04 g, 99%) as a white solid. ¹H NMR (400 MHz, DMSO-d₆) δ 13.83 (br. s., 1H), 12.48 (br. s., 1H), 7.33 (s, 1H), 7.24 (t, J=8.1 Hz, 1H), 7.13-7.08 (m, 3H), 4.09 (s, 3H), 1.90 (s, 6H). ESI-MS m/z calc. 421.0612, found 422.1 (M+1)⁺; Retention time: 4.04 minutes (LC method Y).

### Step 7: 5-[[4-[(2R)-2-Amino-4-methyl-pentoxy]-6-(2,6-dimethylphenyl)pyrimidin-2-yl]sulfamoyl]-2-methyl-pyrazole-3-carboxylic acid

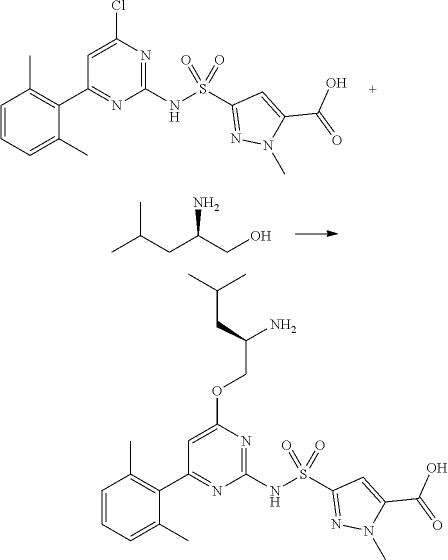

5-[[4-chloro-6-(2,6-dimethylphenyl)pyrimidin-2-yl]sulfamoyl]-2-methyl-pyrazole-3-carboxylic acid (250 mg, 0.5926 mmol) and (2R)-2-amino-4-methyl-pentan-1-ol (100 μL) were combined in THE (1.3 mL) and stirred until the reaction mixture became homogeneous. Sodium tert-butoxide (250 mg, 2.601 mmol) was added and the reaction mixture became warm to the touch and was stirred for 10 minutes without external heating. The reaction mixture was then partitioned between 1M HCl and ethyl acetate. The layers were separated and the aqueous was extracted an additional 3× with ethyl acetate. A substantial amount of product appeared to remain in the aqueous layer, so it was diluted with brine and extracted an additional 5× with ethyl acetate. The combined organics were dried over sodium sulfate and concentrated to give as an off-white solid, which was used in the next step without additional purification. 5-[[4-[(2R)-2-amino-4-methyl-pentoxy]-6-(2,6-dimethylphenyl)pyrimidin-2-yl]sulfamoyl]-2-methyl-pyrazole-3-carboxylic acid (hydrochloride salt) (317 mg, 99%) ESI-MS m/z calc. 502.19983, found 503.3 (M+1)⁺; Retention time: 0.43 minutes (LC method D).

### Example R: Preparation of 5-[[4-[(2R)-2-amino-4,4-dimethyl-pentoxy]-6-(2,6-dimethylphenyl)pyrimidin-2-yl]sulfamoyl]-1-methyl-pyrazole-3-carboxylic acid

### Step 1: Methyl 5-[(4-methoxyphenyl)methylsulfanyl]-1-methyl-pyrazole-3-carboxylate

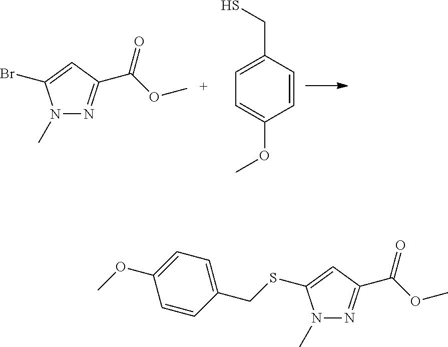

To a sealed tube was added methyl 5-bromo-1-methyl-pyrazole-3-carboxylate (4.71 g, 21.503 mmol), (4-methoxyphenyl)methanethiol (3.32 g, 21.526 mmol) and diisopropylethylamine (5.5650 g, 7.5 mL, 43.058 mmol) in dioxane (100 mL). The mixture was sparged with nitrogen gas for 15 minutes, then added Xantphos (1.24 g, 2.1430 mmol) and Pd2dba3 (980 mg, 1.0702 mmol). The tube was capped and heated in an oil bath set at 100° C. for 5 hours. Once cooled to room temperature, the reaction mixture was transferred to a 1.0-L separatory funnel with water (350 mL) and the aqueous layer was extracted with ethyl acetate (1×300 mL, 1×200 mL). The combined organic layers were washed with brine, dried over sodium sulfate, filtered and concentrated under reduced pressure. The residue was purified by silica gel chromatography on a 220-g column, eluting from 0% to 40% ethyl acetate in heptanes, to afford methyl 5-[(4-methoxyphenyl)methylsulfanyl]-1-methyl-pyrazole-3-carboxylate (5.2 g, 83%) as a pale-yellow solid. ESI-MS m/z calc. 292.0882, found 293.1 (M+1)⁺; Retention time: 1.94 minutes, LC method K.

### Step 2: Methyl 5-chlorosulfonyl-1-methyl-pyrazole-3-carboxylate

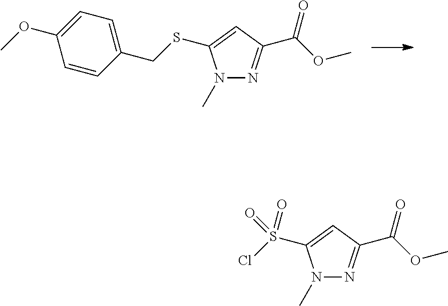

A solution of methyl 5-[(4-methoxyphenyl)methylsulfanyl]-1-methyl-pyrazole-3-carboxylate (4.74 g, 16.213 mmol) in acetic acid (50 mL) and water (25 mL) was treated with N-chlorosuccinimide (6.6 g, 49.426 mmol) at room temperature for 1.5 hours. The reaction was then quenched by adding to a 2.0-L separatory funnel containing cold water (1.5 L) and the aqueous layer was extracted with MTBE (3×250 mL). The combined organic layers were washed with cold water (300 mL), brine, dried over sodium sulfate, filtered and concentrated under reduced pressure. The residue was purified by silica gel chromatography on a 220-g column eluting from 0% to 40% ethyl acetate in heptanes to afford methyl 5-chlorosulfonyl-1-methyl-pyrazole-3-carboxylate (3.62 g, 90%) as a colorless oil. ¹H NMR. ¹H NMR (300 MHz, CDCl₃) δ 7.50 (s, 1H), 4.30 (s, 3H), 3.96 (s, 3H). ESI-MS m/z calc. 237.9815, found 239.0 (M+1)⁺; Retention time: 1.81 minutes, LC method K.

### Step 3: Methyl 5-[[4-chloro-6-(2,6-dimethylphenyl)pyrimidin-2-yl]sulfamoyl]-1-methyl-pyrazole-3-carboxylate

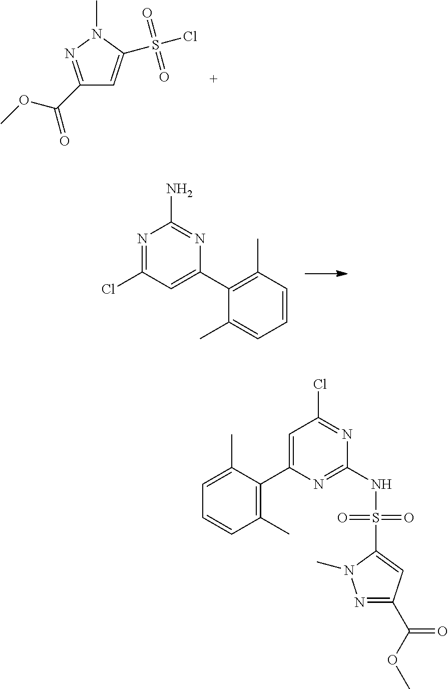 [SMILES: COC(=O)c1cc(S(=O)(=O)Cl)n(C)n1 ; Cc1cccc(C)c1-c1cc(Cl)nc(N)n1 ; COC(=O)c1cc(S(=O)(=O)Nc2nc(Cl)cc(-c3c(C)cccc3C)n2)n(C)n1]

4-chloro-6-(2,6-dimethylphenyl)pyrimidin-2-amine (7.65 g, 32.735 mmol) was dissolved in THE (140 mL) and cooled in an ice bath under stirring and nitrogen. To the cold solution, methyl 5-chlorosulfonyl-1-methyl-pyrazole-3-carboxylate (6.24 g, 26.147 mmol) in solution in THE (45 mL) was added. At 0° C., sodium tert-butoxide (18.5 mL of 40% w/v, 66.584 mmol) was added dropwise (the color was colorless before and yellow after the addition) and the reaction was stirred at room temperature for two hour. The reaction was quenched with HCl 1 N (50 mL). The reaction was diluted with water (150 mL) and EtOAc (250 mL). The organic phase was isolated, and the aqueous phase was extracted with EtOAc (200 mL). The organic phases were combined and washed with water (100 mL and brine (100 mL). The organic phase was dried over sodium sulfate, filtered, and concentrated. The crude was purified by chromatography on silica gel, 120 g, eluted with EtOAc-heptane 5% to 35% to give methyl 5-[[4-chloro-6-(2,6-dimethylphenyl)pyrimidin-2-yl]sulfamoyl]-1-methyl-pyrazole-3-carboxylate (9.15 g, 80%) as a beige solid. ESI-MS m/z calc. 435.0768, found 436.1 (M+1)⁺; Retention time: 1.98 minutes, LC method K.

### Step 4: 5-[[4-Chloro-6-(2,6-dimethylphenyl)pyrimidin-2-yl]sulfamoyl]-1-methyl-pyrazole-3-carboxylic acid

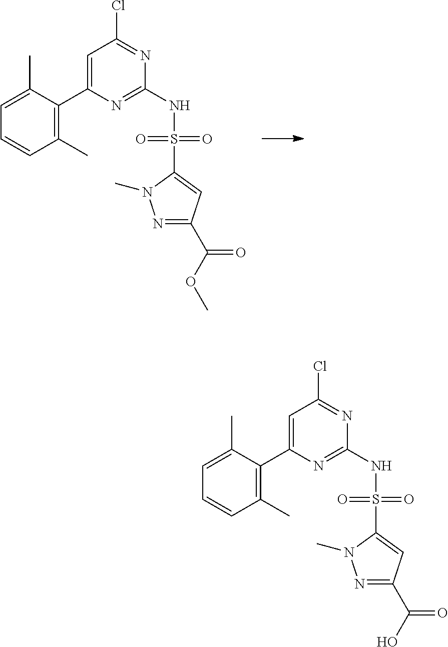 [SMILES: COC(=O)c1cc(S(=O)(=O)Nc2nc(Cl)cc(-c3c(C)cccc3C)n2)n(C)n1 ; Cc1cccc(C)c1-c1cc(Cl)nc(NS(=O)(=O)c2cc(C(=O)O)nn2C)n1]

A mixture of methyl 5-[[4-chloro-6-(2,6-dimethylphenyl)pyrimidin-2-yl]sulfamoyl]-1-methyl-pyrazole-3-carboxylate (832 mg, 1.9088 mmol) in THE (25 mL) and water (25 mL) was treated with lithium hydroxide hydrate (240 mg, 5.7192 mmol) and stirred vigorously at room temperature for 2.5 hours. Most of the THF was removed under reduced pressure, and the remaining aqueous layer was transferred to a 250-mL separatory funnel with water (100 mL) and the aqueous layer was washed with DCM (50 mL). The aqueous layer was acidified to a pH of about 4 using solid citric acid and extracted with ethyl acetate (3×50 mL). The combined organic layers were washed with water (50 mL), brine (50 mL), dried over sodium sulfate, filtered and concentrated under reduced pressure to afford 5-[[4-chloro-6-(2,6-dimethylphenyl)pyrimidin-2-yl]sulfamoyl]-1-methyl-pyrazole-3-carboxylic acid (719 mg, 86%) as a white solid. ¹H NMR (300 MHz, DMSO-d₆) δ 13.14 (br. s., 2H), 7.37 (s, 1H), 7.31-7.22 (m, 1H), 7.18-7.08 (m, 3H), 3.99 (s, 3H), 1.93 (s, 6H). ESI-MS m/z calc. 421.0612, found 422.1 (M+1)⁺; Retention time: 2.62 minutes, LC method U.

### Step 5: 5-[[4-[(2R)-2-Amino-4,4-dimethyl-pentoxy]-6-(2,6-dimethylphenyl)pyrimidin-2-yl]sulfamoyl]-1-methyl-pyrazole-3-carboxylic acid

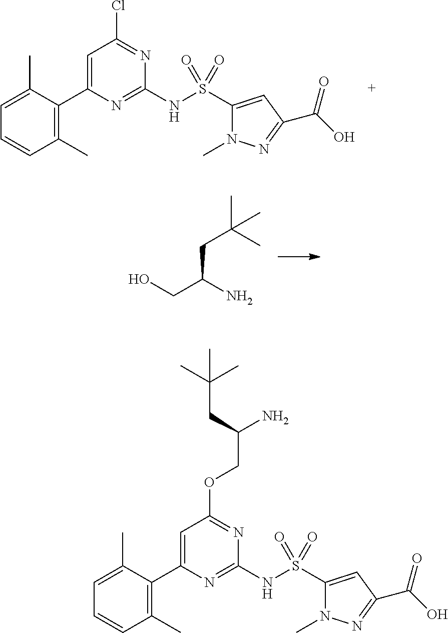

5-[[4-Chloro-6-(2,6-dimethylphenyl)pyrimidin-2-yl]sulfamoyl]-1-methyl-pyrazole-3-carboxylic acid (1.50 g, 3.556 mmol) and (2R)-2-amino-4,4-dimethyl-pentan-1-ol (hydrochloride salt) (656 mg, 3.912 mmol) were combined and dissolved/suspended in THE (12 mL). Solid sodium tert-butoxide (1.71 g, 17.79 mmol) was added in gradual portions over 2 minutes. The reaction mixture was allowed to stir at room temperature for 2 hours. The reaction was quenched with the addition of aqueous HCl (75 mL, 1 M). It was then extracted with EtOAc (3×75 mL). The organic layers were combined, washed with brine (1×100 mL), dried over sodium sulfate, filtered and concentrated under reduced pressure. The crude product was chromatographed on a 24-gram silica gel column eluting with a 0-20% MeOH/DCM gradient over 40 minutes; product eluted at 10% MeOH. The obtained white solid was dissolved into MeOH/DCM, and HCl (800 μL of 4 M, 3.200 mmol) in dioxane was added. After brief stirring, volatiles were removed under reduced pressure to provide 5-[[4-[(2R)-2-amino-4,4-dimethyl-pentoxy]-6-(2,6-dimethylphenyl)pyrimidin-2-yl]sulfamoyl]-1-methyl-pyrazole-3-carboxylic acid (hydrochloride salt) (1.112 g, 57%) was obtained as a pinkish-white solid. ¹H NMR (400 MHz, DMSO-d₆) δ 8.15 (s, 2H), 7.32 (t, J 7.6 Hz, 1H), 7.19 (s, 1H), 7.17 (s, 1H), 7.12 (s, 1H), 6.33 (s, 1H), 4.31 (dd, J=11.9, 3.1 Hz, 1H), 4.13 (d, J=4.1 Hz, 1H), 4.03 (s, 3H), 3.57 (s, 1H), 2.13 (s, 6H), 1.63-1.47 (m, 2H), 0.95 (s, 9H). ESI-MS m/z calc. 516.2155, found 517.2 (M+1)⁺; Retention time: 1.16 minutes (LC method A).

### Example S: Preparation of 3-[[4-[(2R)-2-amino-4-hydroxy-4-methyl-pentoxy]-6-(2,6-dimethylphenyl)pyrimidin-2-yl]sulfamoyl]benzoic acid

### Step 1: Benzyl 2-[(4R)-2-oxooxazolidin-4-yl]acetate

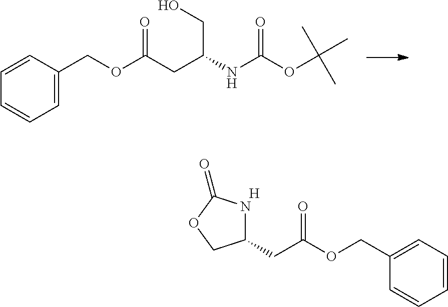

To a solution of benzyl (3R)-3-(tert-butoxycarbonylamino)-4-hydroxy-butanoate (27.8 g, 89.864 mmol)benzyl (3R)-3-(tert-butoxycarbonylamino)-4-hydroxy-butanoate (27.8 g, 89.864 mmol) in 1,2-dichloroethane (250 mL) was added pyridine (65.526 g, 67 mL, 828.40 mmol) and the mixture was cooled to 0-5° C. p-toluenesulfonic anhydride (32.263 g, 98.850 mmol) was added and the mixture was warmed to room temperature and stirred for 2 hours and then heated to 90° C. for 2 hours. The mixture was cooled, diluted with dichloromethane (500 mL) and washed with 1N HCl (3×200 mL). The combined aqueous layers were back extracted with dichloromethane (2×150 mL). The combined organic layers were dried with sodium sulfate, filtered and concentrated to dryness. The crude material was purified by flash chromatography (330 g) using a gradient of 20% to 100% ethyl acetate in heptane to afford enantiopure benzyl 2-[(4R)-2-oxooxazolidin-4-yl]acetate (18.11 g, 86%) as a white solid. ¹H NMR (400 MHz, CDCl₃) δ 7.44-7.31 (m, 5H), 5.58 (br. s., 1H), 5.16 (s, 2H), 4.56 (t, J 8.6 Hz, 1H), 4.25 (qd, J 7.0, 5.9 Hz, 1H), 4.06 (dd, J 8.9, 5.7 Hz, 1H), 2.76-2.63 (m, 2H). ESI-MS m/z calc. 235.0845, found 236.2 (M+1)+, 471.2 (2M+H)⁺; Retention time: 1.49 minutes; LC method X.

### Step 2: (4R)-4-(2-Hydroxy-2-methyl-propyl)oxazolidin-2-one

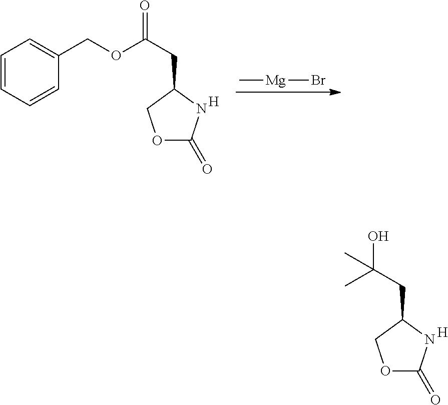

Bromo(methyl)magnesium in diethyl ether (105 mL of 3 M, 315.00 mmol) was added to a mixture of toluene (150 mL) and THE (150 mL) at −20° C. A warm THE (80 mL) solution of benzyl 2-[(4R)-2-oxooxazolidin-4-yl]acetate (18.1 g, 76.944 mmol) was then added dropwise maintaining the temperature below −10° C. The mixture was warm up to room temperature and stirred for 18 hours. The mixture was added via canula to a solution of acetic acid (85 mL) in water (440 mL) at 0° C. The resultant mixture was stirred for 1 hour at room temperature. The layers were separated. The aqueous layer was saturated with brine (200 mL) and further extracted with 2-methyltetrahydrofuran (3×250 mL) and with ethanol/chloroform (½, 3×330 mL). The combined organic extracts were dried over anhydrous sodium sulfate, filtered and concentrated. The residue was co-evaporated with heptanes (4×100 mL). The crude material was purified in two equal batches by flash chromatography (330 g) eluting with 6% isopropanol in dichloromethane) to give (4R)-4-(2-hydroxy-2-methyl-propyl)oxazolidin-2-one (8.88 g, 69%) as an off-white solid. ¹H NMR (400 MHz, DMSO-d₆) δ 7.36 (s, 1H), 4.45-4.38 (m, 1H), 4.36 (s, 1H), 4.00-3.91 (m, 2H), 1.68-1.54 (m, 2H), 1.10 (s, 6H). ESI-MS m/z calc. 159.0895, found 160.2 (M+1)⁺; Retention time: 0.77 minutes, LC method X.

### Step 3: (2R)-2-Amino-4-methyl-pentane-1,4-diol

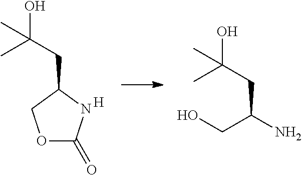

A mixture of (4R)-4-(2-hydroxy-2-methyl-propyl)oxazolidin-2-one (904 mg, 4.2592 mmol) and barium hydroxide octahydrate (4.03 g, 12.775 mmol) in ethanol (20 mL) and water (20 mL) was stirred at 90-95° C. for 4 hours. After cooling down to room temperature, dry ice (˜7 g) was added and the mixture was stirred vigorously for 2 days. The suspension was filtered over a Celite pad and rinsed with ethanol (20 mL). The filtrate was diluted with toluene and concentrated under reduced pressure to provide (2R)-2-amino-4-methyl-pentane-1,4-diol (780 mg) which was used without further purification for the next step. ¹H NMR (400 MHz, DMSO-d₆) δ 5.12 (br. s., 2H), 3.30-3.16 (m, 2H), 2.94 (dd, J 9.0, 3.4 Hz, 1H), 1.83 (s, 2H), 1.49-1.40 (m, 1H), 1.33-1.21 (m, 1H), 1.11 (d, J 11.0 Hz, 6H). ESI-MS m/z calc. 133.1103, found 134.4 (M+1)⁺; Retention time: 0.21 minutes, LC method X.

### Step 4: 3-[[4-[(2R)-2-Amino-4-hydroxy-4-methyl-pentoxy]-6-(2,6-dimethylphenyl)pyrimidin-2-yl]sulfamoyl]benzoic acid

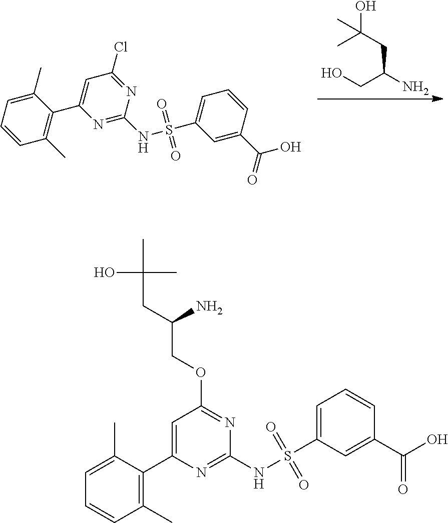

To a solution of (2R)-2-amino-4-methyl-pentane-1,4-diol (567 mg, 4.2571 mmol) and 3-[[4-chloro-6-(2,6-dimethylphenyl)pyrimidin-2-yl]sulfamoyl]benzoic acid (1.5 g, 3.5897 mmol) in tetrahydrofuran (6 mL) was slowly added sodium tert-butoxide in tetrahydrofuran (7.2 mL of 2 M, 14.400 mmol) and the mixture was stirred at room temperature for one hour. The reaction was partitioned between ethyl acetate (30 mL) and 1 N hydrochloric acid (30 mL). The aqueous phase was extracted with ethyl acetate (2×20 mL) and 2-methyltetrahydrofuran (4×30 mL). The combined organic layers were dried over sodium sulfate, filtered and concentrated to dryness. The residue was triturated with ethyl acetate (20 mL), the precipitate was filtered and washed with ethyl acetate (2×10 mL). The product was further dried under vacuum to afford 3-[[4-[(2R)-2-amino-4-hydroxy-4-methyl-pentoxy]-6-(2,6-dimethylphenyl)pyrimidin-2-yl]sulfamoyl]benzoic acid (hydrochloride salt) (1.62 g, 80%) as a pale-yellow solid. ¹H NMR (400 MHz, DMSO-d₆) δ 13.07 (br. s., 2H), 8.43 (s, 1H), 8.14 (d, J 7.8 Hz, 2H), 8.10-8.01 (m, 3H), 7.70 (t, J 7.7 Hz, 1H), 7.32-7.22 (m, 1H), 7.13 (d, J 7.6 Hz, 2H), 6.29 (br. s., 1H), 5.13 (br. s., 1H), 4.36 (dd, J 11.5, 2.9 Hz, 1H), 4.18 (dd, J 11.4, 7.7 Hz, 1H), 3.83-3.70 (m, 1H), 2.02 (s, 6H), 1.71 (d, J 6.4 Hz, 2H), 1.24 (m, 6H). ESI-MS m/z calc. 514.1886, found 515.2 (M+1)⁺; Retention time: 1.3 minutes, LC method X.

### Example T: Preparation of 3-[[4-[(2R)-2-amino-5-hydroxy-5-methyl-hexoxy]-6-(2,6-dimethylphenyl)pyrimidin-2-yl]sulfamoyl]benzoic acid

### Step 1: Benzyl (4R)-4-(tert-butoxycarbonylamino)-5-hydroxy-pentanoate

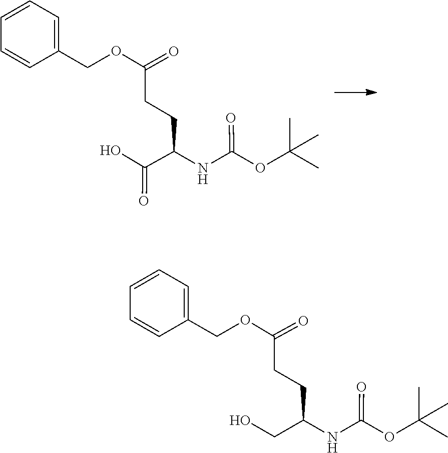 [SMILES: CC(C)(C)OC(=O)N[C@H](CCC(=O)OCc1ccccc1)C(=O)O ; CC(C)(C)OC(=O)N[C@@H](CO)CCC(=O)OCc1ccccc1]

(2R)-5-Benzyloxy-2-(tert-butoxycarbonylamino)-5-oxo-pentanoic acid (10 g, 29.641 mmol) was dissolved in dimethoxyethane (30 mL) and the solution was cooled to −15° C. N-methylmorpholine (3.0360 g, 3.3 mL, 30.016 mmol) was added followed by a slow addition of isobutyl chloroformate (4.1067 g, 3.9 mL, 30.069 mmol) such that the reaction temperature was kept below −10° C. The mixture was stirred for 30 minutes. The solids were quickly filtered and washed with dimethoxyethane (30 mL). The filtrate was cooled to −40° C. and a solution of sodium borohydride (1.45 g, 38.327 mmol) in water (15 mL) was added slowly such that the reaction temperature was maintained between −30° C. and −15° C. The mixture was stirred for 15 minutes. Water (180 mL) was then added dropwise at −15° C. and the temperature was slowly raised to 5° C. while controlling the gas evolution. The suspension was filtered and washed with water (300 mL). The solid was dissolved in dichloromethane (100 mL) and transferred in a separatory funnel. Phases were separated, the organic phase was dried over sodium sulfate, filtered and evaporated to dryness to give benzyl (4R)-4-(tert-butoxycarbonylamino)-5-hydroxy-pentanoate (7.98 g, 83%) as a white solid. ¹H NMR (400 MHz, CDCl₃) δ 7.42-7.30 (m, 5H), 5.13 (s, 2H), 4.81 (br. s., 1H), 3.65 (br. s., 2H), 3.60-3.51 (m, 1H), 2.57-2.36 (m, 3H), 1.98-1.87 (m, 1H), 1.86-1.73 (m, 1H), 1.44 (s, 9H). ESI-MS m/z calc. 323.1733, found 224.4 (M-99)⁺; Retention time: 1.696 minutes, LC method X.

### Step 2: Benzyl 3-[(4R)-2-oxooxazolidin-4-yl]propanoate

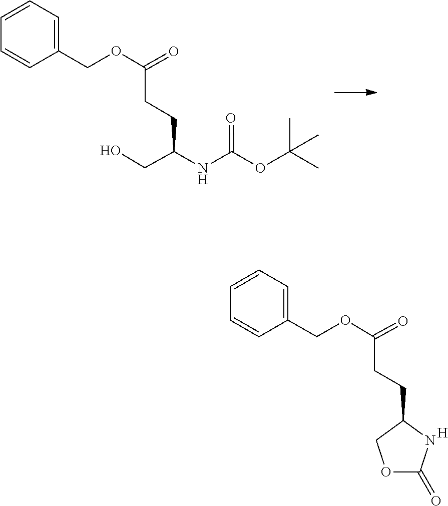

To a solution of benzyl (4R)-4-(tert-butoxycarbonylamino)-5-hydroxy-pentanoate (7.98 g, 24.652 mmol) in dichloroethane (80 mL) was added pyridine (48.900 g, 50 mL, 618.21 mmol). p-toluenesulfonic anhydride (8.65 g, 25.972 mmol) was then added and the mixture was stirred at room temperature for 1 hour and then heated to 90° C. for 2 hours. The mixture was cooled, diluted with dichloromethane (150 mL) and washed with 1N HCl (3×100 mL). The combined organic layers were washed with brine, dried with sodium sulfate and the solvents were removed in vacuo. The residue was purified by silica-gel column chromatography on a 80 g column, eluting from 20% to 80% of EtOAc in heptane to yield benzyl 3-[(4R)-2-oxooxazolidin-4-yl]propanoate (4.85 g, 77%) as a pale brown oil that slowly crystalized over time. ¹H NMR (400 MHz, CDCl₃) δ 7.43-7.30 (m, 5H), 6.15 (br. s., 1H), 5.13 (s, 2H), 4.48 (t, J 8.4 Hz, 1H), 4.02 (dd, J 8.6, 6.1 Hz, 1H), 3.97-3.88 (m, 1H), 2.45 (t, J 7.3 Hz, 2H), 2.00-1.85 (m, 2H). ESI-MS m/z calc. 249.1001, found 250.2 (M+1)⁺; Retention time: 1.511 minutes, LC method X.

### Step 3: (4R)-4-(3-Hydroxy-3-methyl-butyl)oxazolidin-2-one

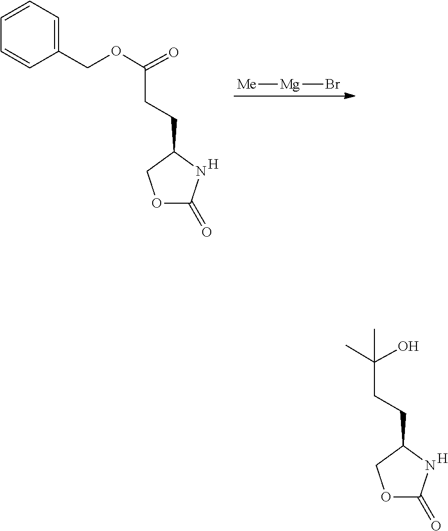

Methylmagnesium bromide (26 mL of 3 M, 78.000 mmol) in diethyl ether was added to a mixture of toluene (42 mL) and tetrahydrofuran (42 mL) at −20° C. A warm tetrahydrofuran (22 mL) solution of benzyl 3-[(4R)-2-oxooxazolidin-4-yl]propanoate (4.85 g, 19.457 mmol) was then added dropwise maintaining the temperature below −10° C. The mixture was warmed up to room temperature and stirred for 2 hours. The reaction mixture was cooled to 0° C., quenched with a 10% aqueous acetic acid solution (50 mL) and the resultant mixture was stirred for 1 hour at room temperature. The layers were separated. The aqueous layer was extracted with methyl-THF (3×100 mL) and then with dichloromethane (2×100 mL). The organic phases were combined, dried on anhydrous sodium sulfate, filtered and concentrated under reduced pressure. The residue was purified by silica-gel column chromatography on a 50 g and 120 g column, eluting from 0 to 15% of isopropanol in dichloromethane to afford (4R)-4-(3-hydroxy-3-methyl-butyl)oxazolidin-2-one (1.73 g, 51%) as a white solid. ¹H NMR (400 MHz, CDCl₃) δ 6.05 (br. s., 1H), 4.50 (t, J 8.4 Hz, 1H), 4.03 (dd, J 8.4, 6.2 Hz, 1H), 3.95-3.81 (m, 1H), 1.76-1.64 (m, 2H), 1.59-1.44 (m, 3H), 1.25 (s, 6H). ESI-MS m/z calc. 173.1052, found 174.2 (M+1)⁺; Retention time: 0.95 minutes, LC method X.

### Step 4: (2R)-2-Amino-5-methyl-hexane-1,5-diol

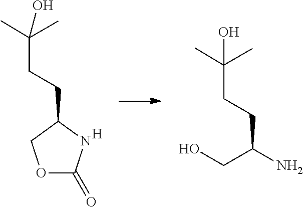

A mixture of (4R)-4-(3-hydroxy-3-methyl-butyl)oxazolidin-2-one (307 mg, 1.7724 mmol), barium hydroxide octahydrate (1.69 g, 5.3572 mmol), ethanol (12 mL) and water (12 mL) was heated at 95° C. to reflux for 2 hours. Reaction mixture was cooled to room temperature before dry ice was slowly added (˜1.8 g) and mixture was stirred vigorously for 2 days. The suspension was filtered over a Celite pad and rinsed with ethanol (˜15 mL). The filtrate was diluted with toluene, co-evaporated three times and concentrated under reduced pressure. Barium salts were observed on the walls of the flask. A minimum of ethanol was added, and the solution was filtered a second time over a Celite pad. The filtrate was concentrated under pressure to provide (2R)-2-amino-5-methyl-hexane-1,5-diol (338.4 mg, 130%) as a yellow oil. The crude was used for the next step without purification. ¹H NMR (400 MHz, DMSO-d₆) δ 3.40-3.28 (m, 1H), 3.25-3.11 (m, 1H), 2.64 (br. s, 1H), 1.81 (s, 2H), 1.51-1.37 (m, 2H), 1.37-1.29 (m, 1H), 1.29-1.18 (m, 1H), 1.06 (d, J 1.0 Hz, 6H). ESI-MS m/z calc. 147.1259, found 148.4 (M+1)⁺; Retention time: 0.22 minutes, LC method X.

### Step 5: 3-[[4-[(2R)-2-Amino-5-hydroxy-5-methyl-hexoxy]-6-(2,6-dimethylphenyl)pyrimidin-2-yl]sulfamoyl]benzoic acid

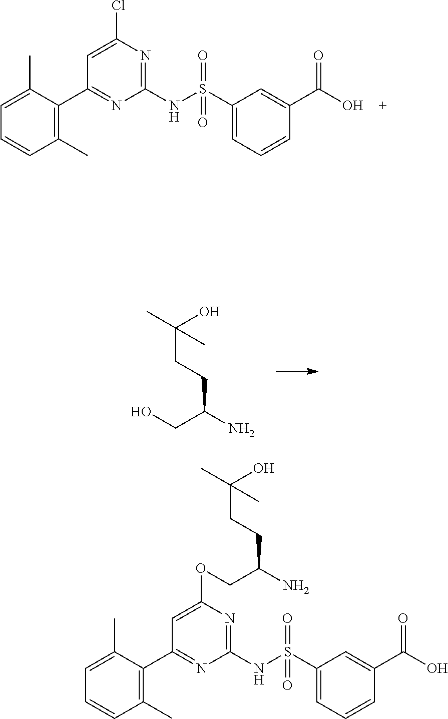

To a solution of 3-[[4-chloro-6-(2,6-dimethylphenyl)pyrimidin-2-yl]sulfamoyl]benzoic acid (371 mg, 0.8878 mmol) and (2R)-2-amino-5-methyl-hexane-1,5-diol (261 mg, 1.7729 mmol) in THE cooled down to 0° C. was slowly added sodium tert-butoxide (375 mg, 3.9020 mmol). After 2 hours sodium tert-butoxide (76 mg, 0.7908 mmol) was slowly added to the reaction and stirred at room temperature. After 2 hours following the addition, sodium tert-butoxide in THE (200 μL of 2 M, 0.4000 mmol) was slowly added and the reaction was stirred at room temperature overnight. The reaction was partitioned between ethyl acetate (6 mL) and hydrochloric acid 1N (6 mL). The aqueous phase was extracted with ethyl acetate (2×6 mL) and 2-methyltetrahydrofuran (3×6 mL). The organic phases were combined, dried over sodium sulfate, filtered and concentrated to dryness. The solid was triturated with ethyl acetate (10 mL) and the precipitate was filtered then washed with ethyl acetate (2×10 mL) to afford 3-[[4-[(2R)-2-amino-5-hydroxy-5-methyl-hexoxy]-6-(2,6-dimethylphenyl)pyrimidin-2-yl]sulfamoyl]benzoic acid (653.4 mg, 139%) as a pale-yellow solid. The crude was used for the next step without purification. ¹H NMR (400 MHz, DMSO-d₆) δ 13.24 (br. s, 1H), 8.43 (s, 1H), 8.19-8.06 (m, 3H), 7.70 (t, J 7.6 Hz, 1H), 7.32-7.19 (m, 1H), 7.18-7.05 (m, 2H), 6.30 (s, 1H), 4.46-4.32 (m, 1H), 4.30-4.18 (m, 1H), 3.53 (s, 1H), 1.99 (s, 6H), 1.78-1.61 (m, 2H), 1.57-1.37 (m, 2H), 1.11 (d, J 7.8 Hz, 6H). ESI-MS m/z calc. 528.2043, found 529.2 (M+1)⁺; Retention time: 1.3 minutes, LC method X.

### Example U: Preparation of 3-[[4-[(2R)-2-amino-4,4-dimethyl-pentoxy]-6-(2,3,6-trimethylphenyl)pyrimidin-2-yl]sulfamoyl]benzoic acid

### Step 1: (2,3,6-Trimethylphenyl) trifluoromethanesulfonate

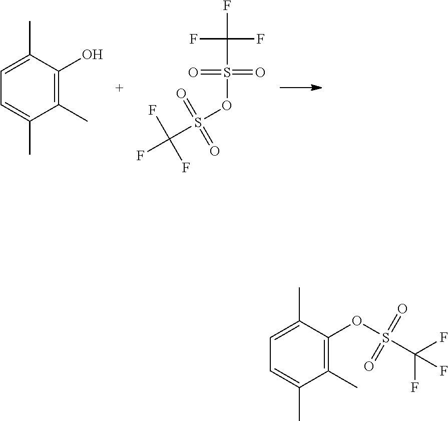

A solution of 2,3,6-trimethylphenol (5 g, 36.713 mmol) in dichloromethane (60 mL) was cooled down to 0° C. and triethylamine (4.4649 g, 6.15 mL, 44.124 mmol) was added. Then trifluoromethanesulfonic anhydride (12.443 g, 7.42 mL, 44.102 mmol) was added dropwise over 15 minutes. Upon addition, the ice bath was removed, and the mixture stirred at room temperature for 16 h. The mixture was diluted with dichloromethane (100 mL), washed with 1M hydrochloric solution (60 mL) and 5% aqueous sodium carbonate (2×50 mL) and brine (50 mL). The organic phase was dried over sodium sulfate, filtered and concentrated to dryness to give (2,3,6-trimethylphenyl) trifluoromethanesulfonate (8.9 g, 90%) as a brown oil. ¹H NMR (400 MHz, CDCl₃) δ 7.06 (d, J 7.8 Hz, 1H), 7.01 (d, J 7.8 Hz, 1H), 2.35 (s, 3H), 2.28 (s, 3H), 2.27 (s, 3H).

### Step 2: 5,5-Dimethyl-2-(2,3,6-trimethylphenyl)-1,3,2-dioxaborinane

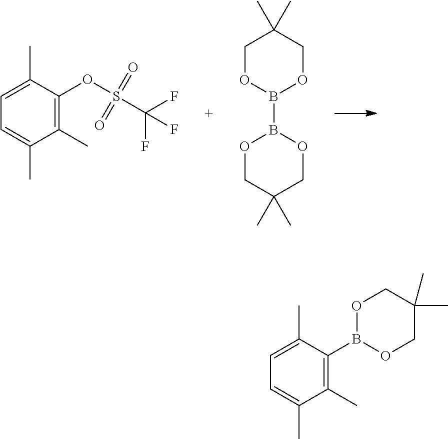 [SMILES: Cc1ccc(C)c(OS(=O)(=O)C(F)(F)F)c1C ; CC1(C)COB(B2OCC(C)(C)CO2)OC1 ; Cc1ccc(C)c(B2OCC(C)(C)CO2)c1C]

A solution of (2,3,6-trimethylphenyl) trifluoromethanesulfonate (8.2 g, 30.538 mmol), bis(neopentyl glycolato)diboron (20.75 g, 91.861 mmol) and potassium acetate (15 g, 152.84 mmol) in 1,4-dioxane (205 mL) was purged by bubbling nitrogen for 15 minutes. [1,1′-Bis(diphenylphosphino)ferrocene]dichloropalladium(II) (2.27 g, 3.1023 mmol) was added and the mixture was stirred for 18 hours at 100-105° C. The mixture was filtered, adsorbed on silica and the product was purified by two successive flash chromatography purifications (on silica 120 g) eluting with 0% to 10% ethyl acetate in heptane to afford 5,5-dimethyl-2-(2,3,6-trimethylphenyl)-1,3,2-dioxaborinane (5.42 g, 72%) as a yellow oil. ¹H NMR (400 MHz, CDCl₃) δ 7.00 (d, J 7.8 Hz, 1H), 6.89 (d, J 7.6 Hz, 1H), 3.82 (s, 4H), 2.37 (s, 3H), 2.31 (s, 3H), 2.22 (s, 3H), 1.14 (s, 6H). ESI-MS m/z calc. 232.1635, found 233.2 (M+1)⁺; Retention time: 4.81 minutes, LC method Y.

### Step 3: tert-Butyl N-[4-chloro-6-(2,3,6-trimethylphenyl)pyrimidin-2-yl]carbamate

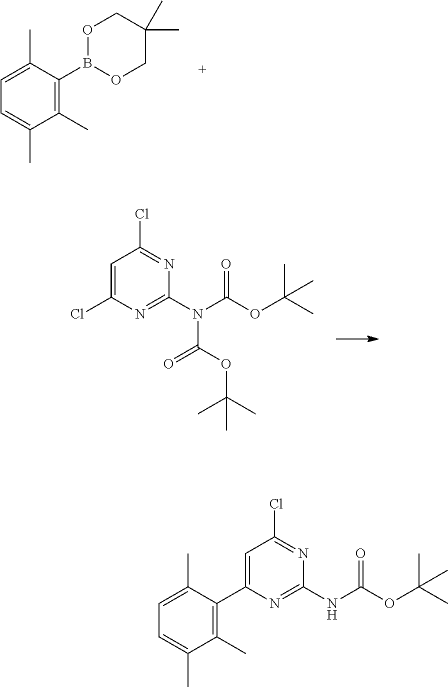 [SMILES: Cc1ccc(C)c(B2OCC(C)(C)CO2)c1C ; CC(C)(C)OC(=O)N(C(=O)OC(C)(C)C)c1nc(Cl)cc(Cl)n1 ; Cc1ccc(C)c(-c2cc(Cl)nc(NC(=O)OC(C)(C)C)n2)c1C]

5,5-Dimethyl-2-(2,3,6-trimethylphenyl)-1,3,2-dioxaborinane (1.00 g, 4.308 mmol) was combined with tert-butyl N-tert-butoxycarbonyl-N-(4,6-dichloropyrimidin-2-yl)carbamate (1.88 g, 5.162 mmol) and dissolved in 1,4-dioxane (17 mL). Water (3 mL) was added followed by barium hydroxide octahydrate (4 g, 12.68 mmol). Pd(dppf)Cl₂ (176 mg, 0.2155 mmol) was added last under nitrogen gas. The reaction mixture was allowed to stir at 80° C. for 1 hour. The reaction mixture was diluted with EtOAc (100 mL) and washed with aqueous HCl (0.5 M, 1×100 mL). The aqueous layer was extracted with EtOAc (1×100 mL). All organic layers were combined and washed with brine (1×75 mL), dried over sodium sulfate, filtered and concentrated under reduced pressure. The crude product was purified by flash chromatography on a 80 gram silica gel column eluting with a 0-30% EtOAc/hexane gradient over 40 minutes; to give tert-butyl N-[4-chloro-6-(2,3,6-trimethylphenyl)pyrimidin-2-yl]carbamate (1.57 g, 105%) as a clear colorless oil. ESI-MS m/z calc. 347.14005, found 292.3 (M-55)⁺; Retention time: 2.04 minutes, LC method A.

### Step 4: 4-Chloro-6-(2,3,6-trimethylphenyl)pyrimidin-2-amine

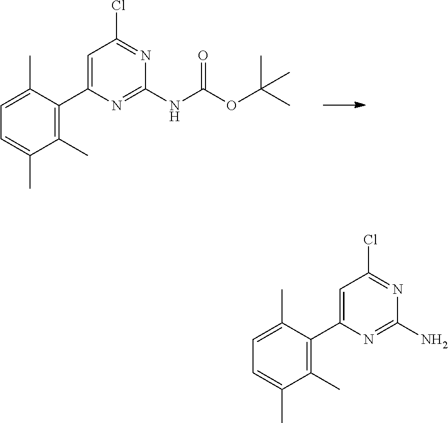 [SMILES: Cc1ccc(C)c(-c2cc(Cl)nc(NC(=O)OC(C)(C)C)n2)c1C ; Cc1ccc(C)c(-c2cc(Cl)nc(N)n2)c1C]

tert-Butyl N-[4-chloro-6-(2,3,6-trimethylphenyl)pyrimidin-2-yl]carbamate (1.57 g, 4.514 mmol) was dissolved in dichloromethane (8 mL). A solution of HCl (5 mL of 4 M, 20.00 mmol) in dioxane was added. The reaction mixture was allowed to stir at room temperature overnight. The obtained slurry was diluted with dichloromethane (75 mL) and washed with aqueous NaOH (1 M, 1×75 mL). The aqueous layer was extracted with dichloromethane (1×75 mL). The organic layers were combined and washed with water (1×100 mL). The organic layer was then dried over sodium sulfate, filtered and concentrated under reduced pressure to give 4-chloro-6-(2,3,6-trimethylphenyl)pyrimidin-2-amine (1.06 g, 95%) as a white waxy semi-solid. ESI-MS m/z calc. 247.08763, found 248.1 (M+1)⁺; Retention time: 1.54 minutes, LC method A.

### Step 5: Methyl 3-[[4-chloro-6-(2,3,6-trimethylphenyl)pyrimidin-2-yl]sulfamoyl]benzoate

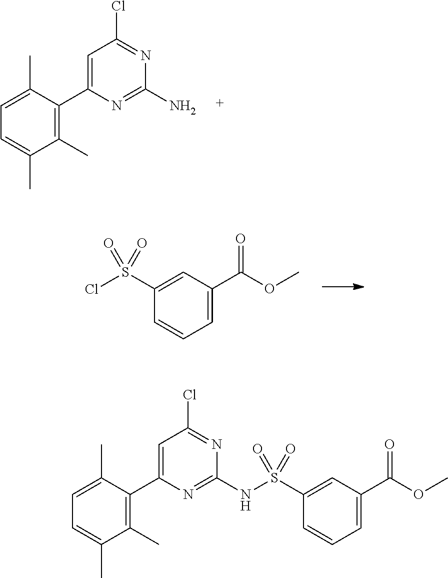 [SMILES: Cc1ccc(C)c(-c2cc(Cl)nc(N)n2)c1C ; COC(=O)c1cccc(S(=O)(=O)Cl)c1 ; COC(=O)c1cccc(S(=O)(=O)Nc2nc(Cl)cc(-c3c(C)ccc(C)c3C)n2)c1]

4-Chloro-6-(2,3,6-trimethylphenyl)pyrimidin-2-amine (1.06 g, 4.279 mmol) was dissolved in tetrahydrofuran (21 mL) and cooled to 0° C. before the addition of sodium hydride (428 mg, 10.70 mmol) (60 wt % dispersion in mineral oil). After stirring for 5 minutes, methyl 3-chlorosulfonylbenzoate (1.51 g, 6.435 mmol) was slowly added dropwise. The reaction mixture was allowed to stir at room temperature for 2 hours. Aqueous HCl (1 M, 75 mL) was added, and the resulting mixture was extracted with EtOAc (2×75 mL). The combined organic layers were washed with brine (1×100 mL), dried over sodium sulfate, filtered and concentrated under reduced pressure. The crude product was chromatographed on a 40 gram silica gel column eluting with a 0-35% EtOAc/hexane gradient over 40 minutes to give methyl 3-[[4-chloro-6-(2,3,6-trimethylphenyl)pyrimidin-2-yl]sulfamoyl]benzoate (631 mg, 33%) as a white solid. ESI-MS m/z calc. 445.0863, found 446.1 (M+1)⁺; Retention time: 1.86 minutes, LC method A.

### Step 6: 3-[[4-Chloro-6-(2,3,6-trimethylphenyl)pyrimidin-2-yl]sulfamoyl]benzoic acid

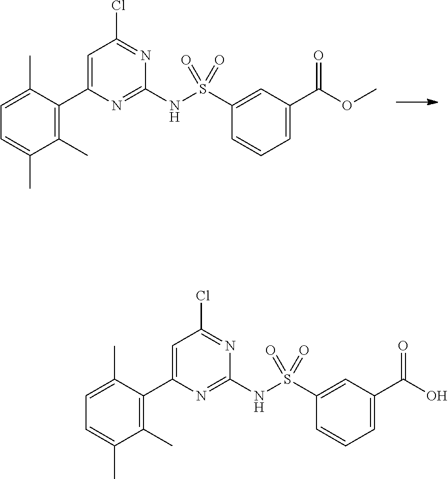 [SMILES: COC(=O)c1cccc(S(=O)(=O)Nc2nc(Cl)cc(-c3c(C)ccc(C)c3C)n2)c1 ; Cc1ccc(C)c(-c2cc(Cl)nc(NS(=O)(=O)c3cccc(C(=O)O)c3)n2)c1C]

Methyl 3-[[4-chloro-6-(2,3,6-trimethylphenyl)pyrimidin-2-yl]sulfamoyl]benzoate (330 mg, 0.7400 mmol) was dissolved in tetrahydrofuran (2.8 mL) and cooled to 0° C. An aqueous solution of sodium hydroxide (1.0 mL of 3 M, 3.000 mmol) was added, and the reaction mixture was allowed to stir at 0° C. for 2 hours. The reaction mixture was diluted with aqueous HCl (1 M, 75 mL) and extracted with EtOAc (2×75 mL). The combined organic layers were washed with brine (1×100 mL), dried over sodium sulfate, filtered and concentrated under reduced pressure. 3-[[4-chloro-6-(2,3,6-trimethylphenyl)pyrimidin-2-yl]sulfamoyl]benzoic acid (317 mg, 99%) was obtained as a foaming solid. ¹H NMR (400 MHz, DMSO-d₆) δ 13.39 (s, 1H), 12.42 (s, 1H), 8.42 (t, J 1.9 Hz, 1H), 8.18 (dt, J 7.8, 1.5 Hz, 1H), 8.11 (dt, J 8.0, 1.4 Hz, 1H), 7.68 (t, J 7.8 Hz, 1H), 7.24 (s, 1H), 7.13 (d, J 7.7 Hz, 1H), 6.98 (d, J 7.7 Hz, 1H), 2.20 (s, 3H), 1.75 (s, 3H), 1.67 (s, 3H). ESI-MS m/z calc. 431.07065, found 432.1 (M+1)⁺; Retention time: 1.62 minutes, LC method A.

### Step 7: 3-[[4-[(2R)-2-Amino-4,4-dimethyl-pentoxy]-6-(2,3,6-trimethylphenyl)pyrimidin-2-yl]sulfamoyl]benzoic acid

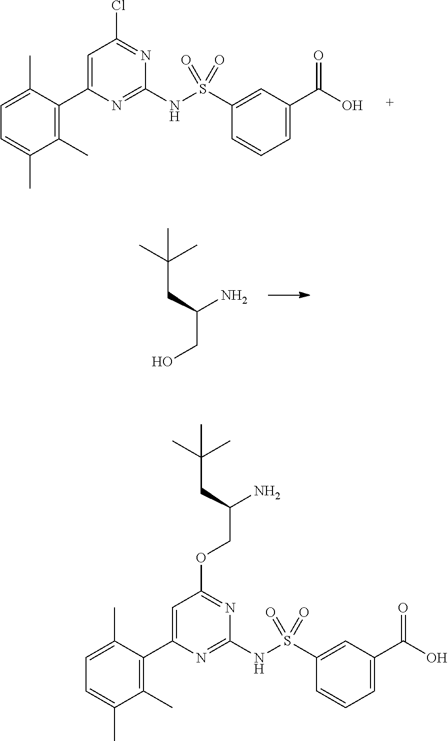

3-[[4-Chloro-6-(2,3,6-trimethylphenyl)pyrimidin-2-yl]sulfamoyl]benzoic acid (317 mg, 0.7340 mmol) and (2R)-2-amino-4,4-dimethyl-pentan-1-ol (hydrochloride salt) (135 mg, 0.8051 mmol) were combined and dissolved/suspended in tetrahydrofuran (5.0 mL). Solid sodium tert-butoxide (353 mg, 3.673 mmol) was added in gradual portions over 2 minutes. The reaction mixture was allowed to stir at room temperature for 2 hours. The reaction mixture was diluted with EtOAc (75 mL). It was then washed with aqueous HCl (0.5 M, 1×75 mL) and brine (1×75 mL). The organic layer was dried over sodium sulfate, filtered and concentrated under reduced pressure. The product was isolated by chromatography on a 12-gram silica gel column eluting with a 0-100% methanol/dichloromethane gradient over 16 minutes. Fractions containing the desired product were combined with HCl (190 μL of 4 M, 0.7600 mmol) and concentrated under reduced pressure to give 3-[[4-[(2R)-2-amino-4,4-dimethyl-pentoxy]-6-(2,3,6-trimethylphenyl)pyrimidin-2-yl]sulfamoyl]benzoic acid (hydrochloride salt) (223 mg, 54%) as a white solid. ESI-MS m/z calc. 526.225, found 527.3 (M+1)⁺; Retention time: 1.14 minutes, LC method A.

### Example V: Preparation of 3-[[4-[(2R)-2-amino-4,4-dimethyl-pentoxy]-6-(2-methyl-1-naphthyl)pyrimidin-2-yl]sulfamoyl]benzoic acid

### Step 1: tert-Butyl N-tert-butoxycarbonyl-N-[4-chloro-6-(2-methyl-1-naphthyl)pyrimidin-2-yl]carbamate

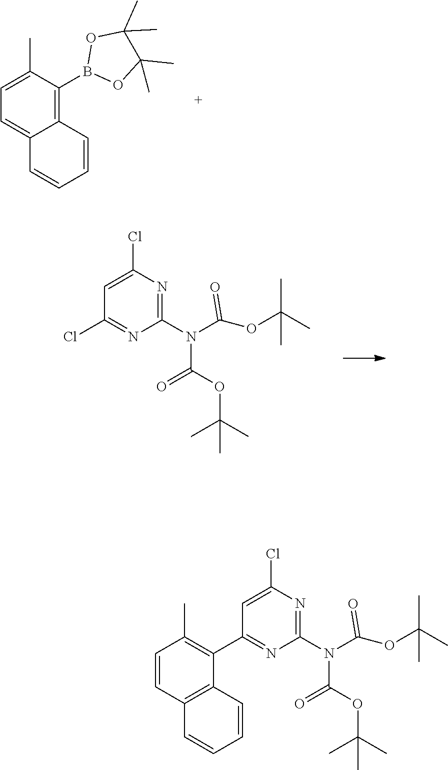 [SMILES: Cc1ccc2ccccc2c1B1OC(C)(C)C(C)(C)O1 ; CC(C)(C)OC(=O)N(C(=O)OC(C)(C)C)c1nc(Cl)cc(Cl)n1 ; Cc1ccc2ccccc2c1-c1cc(Cl)nc(N(C(=O)OC(C)(C)C)C(=O)OC(C)(C)C)n1]

A solution of, 4,4,5,5-tetramethyl-2-(2-methyl-1-naphthyl)-1,3,2-dioxaborolane (2.03 g, 7.4188 mmol), tert-butyl N-tert-butoxycarbonyl-N-(4,6-dichloropyrimidin-2-yl)carbamate (4.651 g, 11.493 mmol) and Cesium carbonate (6.064 g, 18.612 mmol) in a mixture of DME (30 mL) and water (10 mL) was degassed with nitrogen for 5 minutes before adding Pd(dppf)Cl₂ (525.5 mg, 0.7182 mmol) and degassing for another 5 min under nitrogen. The mixture was then stirred at 80° C. for 1 hour. The mixture was then partitioned with DI water (50 mL) and EtOAc (150 mL). The aqueous layer was extracted with EtOAc (2×100 mL). The organic layers were combined, washed with brine, dried over anhydrous sodium sulfate, filtered, and concentrated under reduced pressure. The crude was purified via silica gel column chromatography (40 g column, eluting 0 to 15% EtOAc in hexanes) yielding tert-butyl N-tert-butoxycarbonyl-N-[4-chloro-6-(2-methyl-1-naphthyl)pyrimidin-2-yl]carbamate (3.177 g, 73%) as a yellow solid. ESI-MS m/z calc. 469.1768, found 470.2 (M+1)⁺; Retention time: 4.06 minutes, LC method T.

### Step 2: 4-Chloro-6-(2-methyl-1-naphthyl)pyrimidin-2-amine

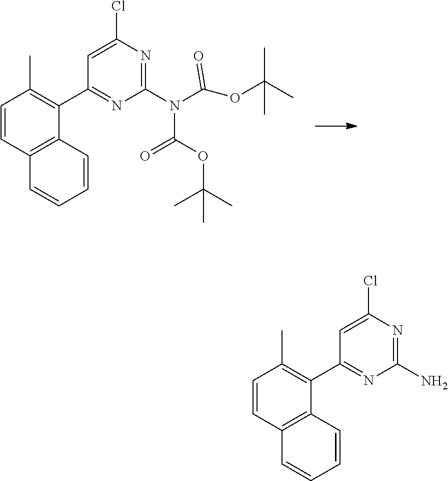 [SMILES: Cc1ccc2ccccc2c1-c1cc(Cl)nc(N(C(=O)OC(C)(C)C)C(=O)OC(C)(C)C)n1 ; Cc1ccc2ccccc2c1-c1cc(Cl)nc(N)n1]

To a solution of tert-butyl N-tert-butoxycarbonyl-N-[4-chloro-6-(2-methyl-1-naphthyl)pyrimidin-2-yl]carbamate (3.342 g, 5.6890 mmol) in DCM (20 mL) at 0° C. was added HCl in dioxane (20 mL of 4 M, 80.000 mmol). The reaction was allowed to reach RT and it was stirred for 3 hours. The reaction was then quenched with aqueous sodium bicarbonate (150 mL) and DCM (100 mL). The aqueous layer was extracted with DCM (2×100 mL). The organic layers were combined, washed with brine, dried over anhydrous sodium sulfate, filtered, and concentrated under reduced pressure to yield 4-chloro-6-(2-methyl-1-naphthyl)pyrimidin-2-amine (2.15 g, 130%) as a yellow solid. ESI-MS m/z calc. 269.072, found 270.0 (M+1)⁺; Retention time: 2.97 minutes, LC method T.

### Step 3: Methyl 3-[[4-chloro-6-(2-methyl-1-naphthyl)pyrimidin-2-yl]sulfamoyl]benzoate

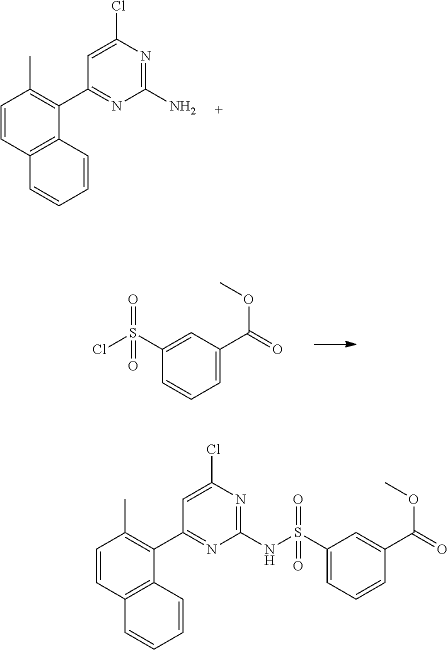 [SMILES: Cc1ccc2ccccc2c1-c1cc(Cl)nc(N)n1 ; COC(=O)c1cccc(S(=O)(=O)Cl)c1 ; COC(=O)c1cccc(S(=O)(=O)Nc2nc(Cl)cc(-c3c(C)ccc4ccccc34)n2)c1]

A solution of crude 4-chloro-6-(2-methyl-1-naphthyl)pyrimidin-2-amine (2.15 g, 7.4130 mmol) in anhydrous THE (28 mL) was cooled to 0° C. Then a solution of methyl 3-chlorosulfonylbenzoate (2.278 g, 9.7078 mmol) in anhydrous THE (35 mL) was added. Lithium tert-amoxide in heptane (1.3724 g, 4.7 mL of 40% w/w, 5.8350 mmol) was then added dropwise. The reaction was brought up to room temperature and stirred for 2 hours. The reaction was then quenched with 1M HCl (50 mL) and EtOAc (100 mL). The aqueous layer was extracted with EtOAc (2×100 mL). The organic layers were combined, washed with brine, dried over anhydrous sodium sulfate, filtered, and concentrated under reduced pressure. The crude was purified via silica gel column chromatography (40 g column, eluting 0 to 35% EtOAc in hexanes) to yield methyl 3-[[4-chloro-6-(2-methyl-1-naphthyl)pyrimidin-2-yl]sulfamoyl]benzoate (2.579 g, 64%) as a white solid. ESI-MS m/z calc. 467.0707, found 468.1 (M+1)⁺; Retention time: 3.39 minutes, LC method T.

### Step 4: 3-[[4-Chloro-6-(2-methyl-1-naphthyl)pyrimidin-2-yl]sulfamoyl]benzoic acid

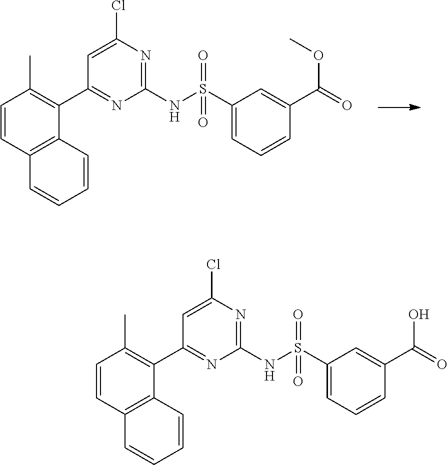 [SMILES: COC(=O)c1cccc(S(=O)(=O)Nc2nc(Cl)cc(-c3c(C)ccc4ccccc34)n2)c1 ; Cc1ccc2ccccc2c1-c1cc(Cl)nc(NS(=O)(=O)c2cccc(C(=O)O)c2)n1]

To a solution of methyl 3-[[4-chloro-6-(2-methyl-1-naphthyl)pyrimidin-2-yl]sulfamoyl]benzoate (2.554 g, 5.4581 mmol) in THE (51 mL) was added aqueous solution of NaOH (11 mL of 2 M, 22.000 mmol). The solution was stirred for 1 hour. The solution was then quenched with 1M HCl (10 mL) and EtOAc (20 mL). The aqueous layer was then extracted with EtOAc (2×20 mL). The organic layers were combined, washed with brine, dried over anhydrous sodium sulfate, filtered, and concentrated under reduced pressure to yield 3-[[4-chloro-6-(2-methyl-1-naphthyl)pyrimidin-2-yl]sulfamoyl]benzoic acid (2.439 g, 87%) as a white solid. ESI-MS m/z calc. 453.055, found 454.0 (M+1)⁺; Retention time: 3.03 minutes, LC method T.

### Step 5: 3-[[4-[(2R)-2-Amino-4,4-dimethyl-pentoxy]-6-(2-methyl-1-naphthyl)pyrimidin-2-yl]sulfamoyl]benzoic acid

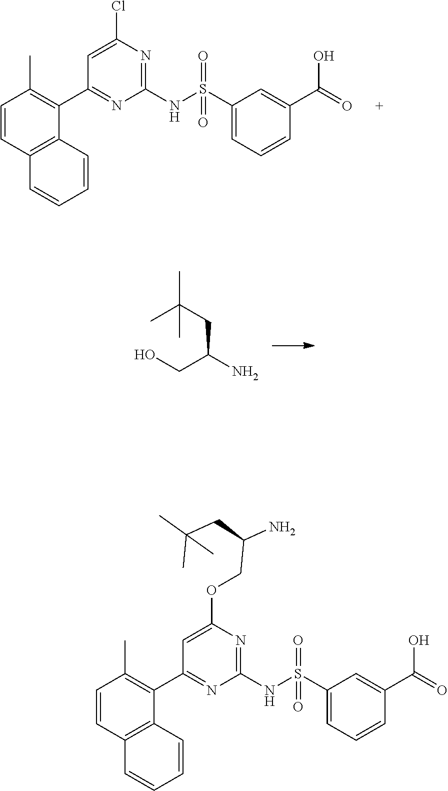

To a solution of 3-[[4-chloro-6-(2-methyl-1-naphthyl)pyrimidin-2-yl]sulfamoyl]benzoic acid (2.412 g, 5.3140 mmol) and (2R)-2-amino-4,4-dimethyl-pentan-1-ol (hydrochloride salt) (1.023 g, 6.1010 mmol) in anhydrous THE (85 mL) was added sodium tert-butoxide (2.054 g, 21.373 mmol). The solution was stirred at RT for 2 hours. The solution was then concentrated under reduced pressure. The residue was then purified via reverse phase HPLC (gradient 25-75% acetonitrile in water buffered by 5 mM HCl) to yield 3-[[4-[(2R)-2-amino-4,4-dimethyl-pentoxy]-6-(2-methyl-1-naphthyl)pyrimidin-2-yl]sulfamoyl]benzoic acid (hydrochloride salt) (1.345 g, 41%) as a white powder. ESI-MS m/z calc. 548.2093, found 549.4 (M+1)⁺; Retention time: 1.88 minutes, LC method W. ¹H NMR (500 MHz, DMSO-d₆) δ 13.43 (s, 1H), 8.48 (d, J 1.8 Hz, 1H), 8.27-8.10 (m, 5H), 7.97 (m, 2H), 7.70 (m, 1H), 7.54-7.42 (m, 3H), 6.43 (s, 1H), 4.37 (d, J 11.8 Hz, 1H), 4.16 (m, 1H), 3.60 (s, 1H), 2.21 (s, 3H), 1.65-1.59 (m, 1H), 1.52 (m, 1H), 0.96 (s, 9H).

### Example W: Preparation of (1R,2R)-2-Amino-1-(4-tert-butylphenyl)propan-1-ol (hydrochloride salt)

### Step 1: tert-Butyl N-[(1R)-1-methyl-2-oxo-ethyl]carbamate

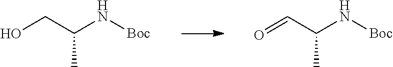

To a solution of tert-butyl N-[(1R)-2-hydroxy-1-methyl-ethyl]carbamate (200 g, 1.141 mol) in DCM (3 L) was added Dess-Martin periodinane (625 g, 1.474 mol) (fine suspension, most into solution, started exotherm, controlled with ice-bath). To the mixture was added water (28 mL, 1.554 mol) slowly added over 0.5 h (exothermic during addition up to 33° C., kept between 20 and 33° C. by cooling with cold water) giving a colorless thick suspension. The suspension was stirred at room temperature for 16 h. The solid was removed by filtration over Celite and washed 3× with 100 mL of DCM. The solvent was removed in vacuo affording an off-white slurry, which was diluted with MTBE (750 mL). The slurry was cooled with an ice-bath and filtered over Celite. The filtrate was washed 3× with sat sodium bicarbonate, brine, dried over magnesium sulfate, filtered and concentrated in vacuo. The semi-solid was re-dissolved in MTBE (300 mL) and diluted with heptane (750 mL). The solution was concentrated in vacuo until a cloud point occurred. The slurry was stirred at ambient temperature for 0.5 h. The precipitate was collected, washed with cold heptane and dried in vacuo at ambient temperature (this solid was product and was therefore kept aside). The filtrate was further concentrated in vacuo until a cloud point occurred. The solution was allowed to stand for 48 h affording a thick off-white slurry. The slurry was filtered, and the filter cake was washed with −50 mL of cold heptane. The filter cake was combined with the solid kept aside earlier and air-dried for 4 h. Product contained approximately 9% residual heptane by ¹H NMR. tert-Butyl N-[(1R)-1-methyl-2-oxo-ethyl]carbamate (95.6 g, 48%), ¹H NMR (500 MHz, DMSO-d₆) δ 9.43 (s, 1H), 7.35 (d, J 6.8 Hz, 1H), 3.86 (t, J 7.2 Hz, 1H), 1.40 (s, 9H), 1.13 (d, J 7.3 Hz, 3H).

### Step 2: tert-Butyl N-[(1R,2R)-2-(4-tert-butylphenyl)-2-hydroxy-1-methyl-ethyl]carbamate

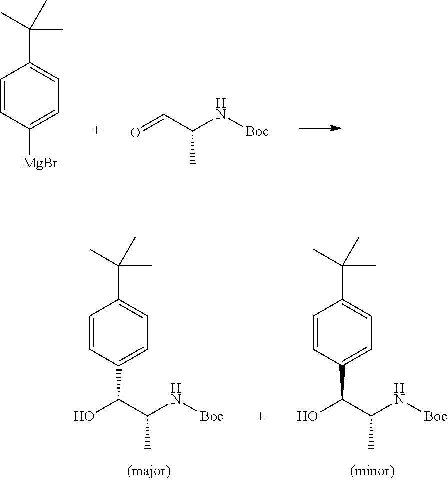

A solution of tert-butyl N-[(1R)-1-methyl-2-oxo-ethyl]carbamate (101.73 g, 587.3 mmol) in MeTHF (500 mL) was added slowly over 1 h to bromo-(4-tert-butylphenyl)magnesium (1300 mL of 1 M, 1.300 mol) (1 M in MeTHF) in a −35° C. cold bath at a rate which maintained an internal temperature between −2° C. and −15° C. After the addition was complete, it was stirred for 5 min, then the mixture was removed from the cold bath and transferred to a room temperature water bath, then stirred for 2.5 h. The mixture was cooled to 0° C., then saturated ammonium chloride (1700 mL) was added (large exotherm) at a rate which maintained an internal temperature of 5° C. Water (500 mL) was added, the organic layer was separated and washed with brine (500 mL), dried over magnesium sulfate, then concentrated under vacuum to give a light yellow oil, tert-butyl N-[(1R,2R)-2-(4-tert-butylphenyl)-2-hydroxy-1-methyl-ethyl]carbamate (266 g, >100% yield), which was used in the next step without further purification. ESI-MS m/z calc. 307.21475, found 308.1 (M+1)⁺; Retention time: 1.86 minutes; LC method A.

### Step 3: (1R,2R)-2-Amino-1-(4-tert-butylphenyl)propan-1-ol (hydrochloride salt)

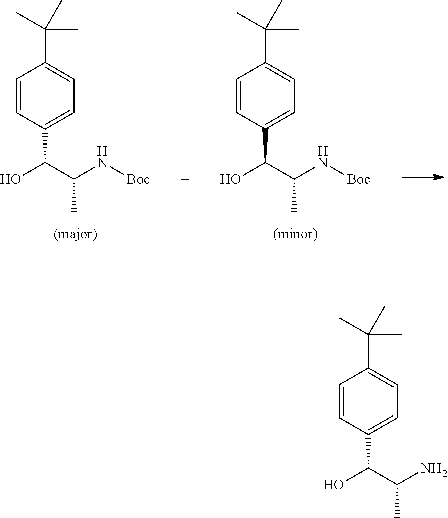 [SMILES: C[C@@H](NC(=O)OC(C)(C)C)[C@H](O)c1ccc(C(C)(C)C)cc1 ; C[C@@H](NC(=O)OC(C)(C)C)[C@@H](O)c1ccc(C(C)(C)C)cc1 ; C[C@@H](N)[C@H](O)c1ccc(C(C)(C)C)cc1]

A solution of tert-butyl N-[(1R,2R)-2-(4-tert-butylphenyl)-2-hydroxy-1-methyl-ethyl]carbamate (180.6 g, 587.5 mmol) in MeOH (250 mL) was added dropwise over 50 min to HCl in dioxane (478 mL of 4 M, 1.912 mol), maintaining a temperature between 18° C. and 23° C., then stirred at room temperature for 2 h. The mixture was concentrated under vacuum to give 267.5 g of residue. This was recrystallized from dioxane, the product was collected by filtration, then rinsed with MeTHF until all the color was removed, giving 75.4 g of product. This was further recrystallized from MeOH/dioxane, which gave (1R,2R)-2-amino-1-(4-tert-butylphenyl)propan-1-ol (hydrochloride salt) (62.65 g, 44%); ¹H NMR (500 MHz, DMSO-d₆) δ 8.10 (s, 3H), 7.39 (d, J 8.2 Hz, 2H), 7.28 (d, J 8.1 Hz, 2H), 6.12 (d, J 3.8 Hz, 1H), 4.50-4.34 (m, 1H), 3.28-3.12 (m, 1H), 1.27 (s, 9H), 0.96 (d, J 6.6 Hz, 3H). ESI-MS m/z calc. 207.16231, found 208.2 (M+1)⁺; Retention time: 1.01 minutes; LC method A.

### V. Synthesis of Compounds 1-1294

### Example 1: Preparation of Compound 1

### Step 1: (2R)-2-[Benzyl(methyl)amino]-N-methoxy-N,4-dimethyl-pentanamide

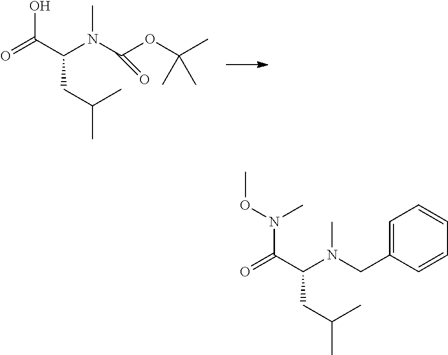 [SMILES: CC(C)C[C@H](C(=O)O)N(C)C(=O)OC(C)(C)C ; CON(C)C(=O)[C@@H](CC(C)C)N(C)Cc1ccccc1]

Stage 1: In a 1-L round-bottomed flask, (2R)-2-[tert-butoxycarbonyl(methyl)amino]-4-methyl-pentanoic acid (21.46 g, 82.23 mmol), DCM (110 mL), DMF (110 mL), N-methoxymethanamine (hydrochloride salt) (11.50 g, 117.9 mmol), DIPEA (68 mL, 390.4 mmol), HOBt (15.97 g, 118.2 mmol) and EDCI (hydrochloride salt) (27.05 g, 118.6 mmol) were added in this order. This solution was stirred at room temperature for 4 h, after which it was diluted with ethyl acetate (1 L). This mixture was washed with 1N NaOH solution (400 mL), 1N HCl solution (2×400 mL), water (400 mL) and saturated aqueous sodium chloride solution (400 mL), then dried over sodium sulfate, filtered, and evaporated in vacuo to give a slightly yellow liquid, corresponding to the Weinreb amide intermediate (˜27 g, >100% yield), ESI-MS m/z calc. 288.2049, found 289.3 (M+1)⁺; Retention time: 1.64 minutes; LC method A.

Stage 2: In a 250-mL round-bottomed flask, the crude product from Stage 1 was dissolved in dioxane (25 mL) and cooled to 0° C. This solution was treated with a dioxane solution of HCl (75 mL of 4.0 M, 300.0 mmol), and the resulting mixture was warmed to room temperature over 4 h. Evaporation of the resulting slurry in vacuo provided an off-white solid, corresponding to the deprotected intermediate (˜28 g, >100% yield).

Stage 3: In a 250-mL round-bottomed flask, the crude product from Stage 2 was dissolved in EtOH (100 mL) and water (25 mL), to which potassium carbonate (35.0 g, 253.2 mmol) and benzyl bromide (11.0 mL, 92.48 mmol) were added. This slurry was stirred at room temperature for 69 h, after which it was filtered over Celite, using MeOH (50 mL) to rinse the potassium carbonate and Celite. The filtrate was evaporated in vacuo and this slurry was taken up in DCM (100 mL), filtered over Celite and evaporated in vacuo. The resulting yellow liquid was then purified by silica gel chromatography (330 g of silica) using a gradient eluent of 1 to 5% MeOH in DCM, then filtered under a flow of nitrogen to give a colorless viscous liquid: (2R)-2-[benzyl(methyl)amino]-N-methoxy-N,4-dimethyl-pentanamide (12.0617 g, 53%); ¹H NMR (400 MHz, dimethylsulfoxide-d₆) δ 7.33-7.25 (m, 4H), 7.25-7.19 (m, 1H), 4.06-3.73 (m, 1H), 3.66 (AB quartet, 2H), 3.59 (s, 3H), 3.11 (s, 3H), 2.21 (s, 3H), 1.68-1.53 (m, 2H), 1.53-1.41 (m, 1H), 0.88 (dd, J 6.7 Hz, 1H). ESI-MS m/z calc. 278.19943, found 279.3 (M+1)⁺; Retention time: 0.88 minutes; LC method A.

### Step 2: (2R)-2-[Benzyl(methyl)amino]-1-(5-tert-butyl-2-pyridyl)-4-methyl-pentan-1-one

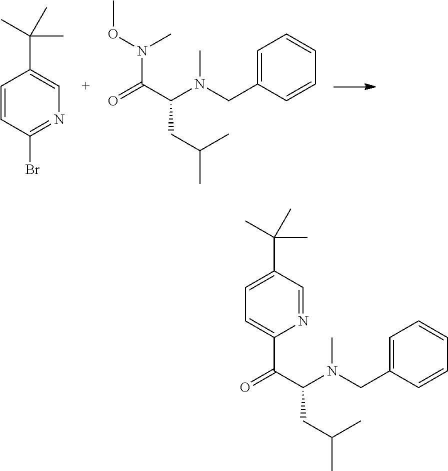 [SMILES: CC(C)(C)c1ccc(Br)nc1 ; CON(C)C(=O)[C@@H](CC(C)C)N(C)Cc1ccccc1 ; CC(C)C[C@H](C(=O)c1ccc(C(C)(C)C)cn1)N(C)Cc1ccccc1]

In a 20-mL microwave vial, 2-bromo-5-tert-butyl-pyridine (350 mg, 1.635 mmol) was dissolved in anhydrous THE (8 mL) and cooled to −78° C. A hexanes solution of nBuLi (700 μL of 2.5 M, 1.750 mmol) was added in one portion, and this mixture was stirred at −78° C. for 10 min. A solution of (2R)-2-[benzyl(methyl)amino]-N-methoxy-N,4-dimethyl-pentanamide (455.3 mg, 1.635 mmol) in anhydrous THE (2 mL) was then added dropwise. This solution was stirred at −78° C. for 5 min and warmed to room temperature over 2 h. The reaction mixture was then quenched with 0.5 N HCl (20 mL) and extracted with ethyl acetate (3×20 mL). The combined organic extracts were washed with water (50 mL) and saturated aqueous sodium chloride solution (50 mL), then dried over sodium sulfate, filtered, and evaporated in vacuo. The resulting yellow oil was purified by silica gel chromatography (40 g of silica) using a gradient eluent of 0 to 40% ethyl acetate in hexanes to give the product as a yellow oil: (2R)-2-[benzyl(methyl)amino]-1-(5-tert-butyl-2-pyridyl)-4-methyl-pentan-1-one (339.0 mg, 59%)¹H NMR (400 MHz, dimethylsulfoxide-d₆) δ 8.80 (dd, J 2.4, 0.8 Hz, 1H), 8.03 (dd, J 8.2, 2.4 Hz, 1H), 7.92 (dd, J=8.3, 0.8 Hz, 1H), 7.28-7.22 (m, 2H), 7.22-7.17 (m, 1H), 7.18-7.14 (m, 2H), 5.11 (t, J 7.1 Hz, 1H), 3.65 (s, 2H), 2.15 (s, 3H), 1.70-1.55 (m, 3H), 1.36 (s, 9H), 0.90 (d, J 6.0 Hz, 6H) ESI-MS m/z calc. 352.25146, found 353.4 (M+1)⁺; Retention time: 1.5 minutes; LC method A.

### Step 3: (1S,2R)-2-[Benzyl(methyl)amino]-1-(5-tert-butyl-2-pyridyl)-4-methyl-pentan-1-ol

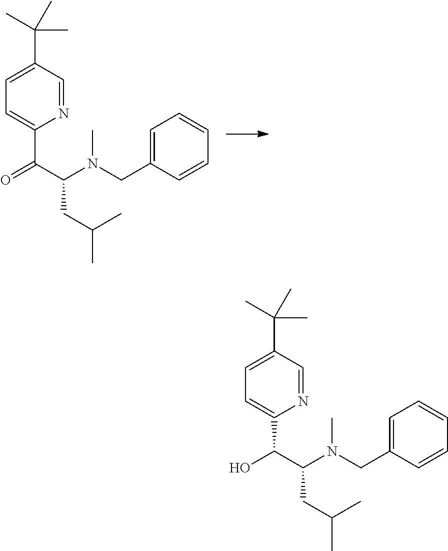 [SMILES: CC(C)C[C@H](C(=O)c1ccc(C(C)(C)C)cn1)N(C)Cc1ccccc1 ; CC(C)C[C@H]([C@H](O)c1ccc(C(C)(C)C)cn1)N(C)Cc1ccccc1]

In a 20-mL vial, (2R)-2-[benzyl(methyl)amino]-1-(5-tert-butyl-2-pyridyl)-4-methyl-pentan-1-one (333.9 mg, 0.9472 mmol) was dissolved in MeOH (2.0 mL), to which sodium borohydride (45.3 mg, 1.197 mmol) was added. This mixture was stirred at room temperature for 10 min, after which it was quenched with 0.5 N HCl solution (5 mL). The mixture was neutralized with 0.5 N NaOH (˜4 mL), then extracted with ethyl acetate (4×5 mL). The combined organic extracts was washed with water (10 mL) and saturated aqueous sodium chloride solution (10 mL), then dried over sodium sulfate, filtered, and evaporated in vacuo to give a slightly yellow viscous gum, (1S,2R)-2-[benzyl(methyl)amino]-1-(5-tert-butyl-2-pyridyl)-4-methyl-pentan-1-ol (303.1 mg, 90%)¹H NMR (400 MHz, dimethylsulfoxide-d₆) δ 8.52 (d, J 2.1 Hz, 1H), 7.79 (dd, J 8.3, 2.6 Hz, 1H), 7.41 (dd, J 8.3, 0.7 Hz, 1H), 7.28-7.15 (m, 3H), 7.14-7.00 (m, 2H), 5.45-4.88 (bs, 1H), 4.70-4.51 (m, 1H), 3.70 (AB quartet, Δδ_{AB}=0.13 ppm, J_{AB}=13.2 Hz, 2H), 3.11-2.84 (m, 1H), 2.19 (s, 3H), 1.59-1.26 (m, 3H), 1.32 (s, 9H), 0.78 (d, J 6.5 Hz, 3H), 0.72 (d, J 6.3 Hz, 3H) ESI-MS m/z calc. 354.26712, found 355.4 (M+1)⁺; Retention time: 1.3 minutes; LC method A.

### Step 4: 3-[[4-[(1S,2R)-1-(5-tert-Butyl-2-pyridyl)-4-methyl-2-(methylamino)pentoxy]-6-(2,6-dimethylphenyl)pyrimidin-2-yl]sulfamoyl]benzoic acid

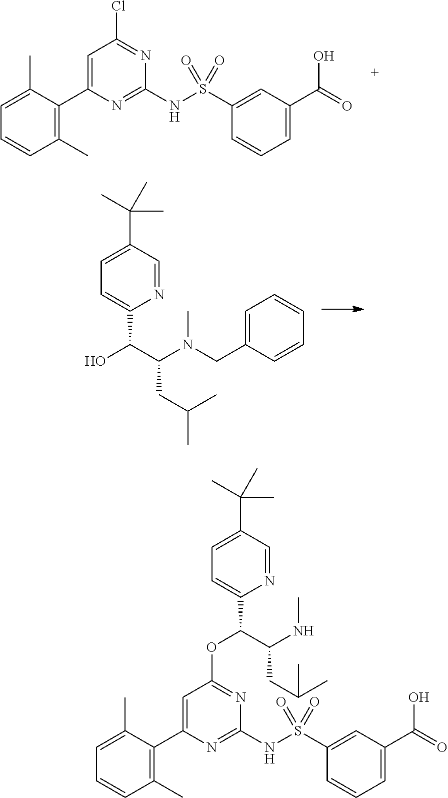

Stage 1: In a 50-mL round-bottomed flask, (1S,2R)-2-[benzyl(methyl)amino]-1-(5-tert-butyl-2-pyridyl)-4-methyl-pentan-1-ol (297.4 mg, 0.8389 mmol) was dissolved in PhMe (5 mL), to which 3-[[4-chloro-6-(2,6-dimethylphenyl)pyrimidin-2-yl]sulfamoyl]benzoic acid (360 mg, 0.8615 mmol) was added. This mixture was evaporated to dryness in vacuo. The resulting solid was dissolved in anhydrous NMP (5.0 mL), and treated with NaH (222 mg of 60% w/w, 5.551 mmol). This slurry was stirred at 70° C. for 30 min, after which it was cooled to room temperature, quenched with 1 N HCl (10 mL), and extracted with ethyl acetate (3×10 mL). The combined organic extracts were washed with water (20 mL) and saturated aqueous sodium chloride solution (20 mL), then dried over sodium sulfate, filtered, and evaporated in vacuo. this gave a light yellow foam, 3-[[4-[(1S,2R)-2-[benzyl(methyl)amino]-1-(5-tert-butyl-2-pyridyl)-4-methyl-pentoxy]-6-(2,6-dimethylphenyl)pyrimidin-2-yl]sulfamoyl]benzoic acid (772 mg, >100%) ESI-MS m/z calc. 735.34546, found 736.6 (M+1)⁺; Retention time: 0.61 minutes; LC method D.

Stage 2: In a 20-mL microwave vial equipped with a magnetic stir bar, the crude product from Stage 1 (772.5 mg, assume 0.8615 mmol if quantitative yield in Stage 1) was dissolved in EtOH (5.0 mL). This solution was sparged with a balloon of hydrogen gas for 5 min. The cap was briefly removed, and Pd/C (107.7 mg of 10% w/w, 0.1012 mmol) was added. This reaction mixture was stirred under a balloon of hydrogen at room temperature for 15 h, after which it was filtered through Celite and rinsed with methanol (10 mL). This solution was evaporated in vacuo to give a yellow solid, 3-[[4-[(1S,2R)-1-(5-tert-butyl-2-pyridyl)-4-methyl-2-(methylamino)pentoxy]-6-(2,6-dimethylphenyl)pyrimidin-2-yl]sulfamoyl]benzoic acid (640 mg, >100%) ESI-MS m/z calc. 645.29846, found 646.6 (M+1)⁺; Retention time: 0.5 minutes; LC method D.

### Step 5: (10S,1IR)-10-(5-tert-Butyl-2-pyridyl)-6-(2,6-dimethylphenyl)-11-isobutyl-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1)

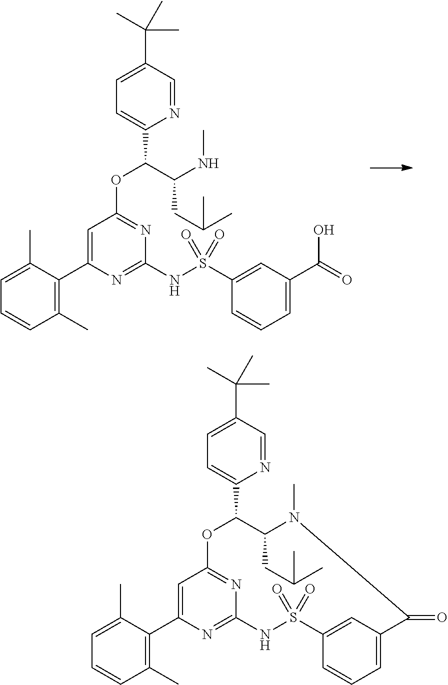

Stage 1: In a 20-mL vial, 3-[[4-[(1S,2R)-1-(5-tert-butyl-2-pyridyl)-4-methyl-2-(methylamino)pentoxy]-6-(2,6-dimethylphenyl)pyrimidin-2-yl]sulfamoyl]benzoic acid (320 mg, 0.4195 mmol) was dissolved in DMF (5.0 mL). DIPEA (400 μL, 2.296 mmol) and Ph₂P(O)—OC₆F₅ (340 mg, 0.8849 mmol) were added, and this solution was stirred at room temperature for 45 min. The reaction mixture was then quenched with 1 N HCl (5 mL), and extracted with ethyl acetate (3×5 mL). The combined organic extracts were washed with water (10 mL) and saturated aqueous sodium chloride solution (10 mL), then dried over sodium sulfate, filtered, and evaporated in vacuo. This crude product was purified by silica gel chromatography (12 g of silica) using a gradient eluent of 1 to 90% ethyl acetate in hexanes to give (2,3,4,5,6-pentafluorophenyl) 3-[[4-[(1S,2R)-1-(5-tert-butyl-2-pyridyl)-4-methyl-2-(methylamino)pentoxy]-6-(2,6-dimethylphenyl)pyrimidin-2-yl]sulfamoyl]benzoate (249.0 mg, 73%) ESI-MS m/z calc. 811.28265, found 812.6 (M+1)⁺; Retention time: 1.81 minutes.

Stage 2: In a 100-mL round-bottomed flask, the purified product from Stage 1 (240 mg, 0.296 mmol) was dissolved in NMP (20 mL), and stirred under nitrogen at 160° C. for 63 h. After this time, the reaction mixture was cooled to room temperature, diluted with water (60 mL), and extracted with ethyl acetate (3×60 mL). The combined organic extracts were washed with water (120 mL) and saturated aqueous sodium chloride solution (120 mL), then dried over sodium sulfate, filtered, and evaporated in vacuo. The resulting brown oil was purified by silica gel chromatography (12 g of silica) using a gradient eluent of 1 to 60% ethyl acetate in hexanes, followed by a second purification by reverse-phase preparative HPLC (C₁₈) to give (10S,11R)-10-(5-tert-butyl-2-pyridyl)-6-(2,6-dimethylphenyl)-11-isobutyl-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (5.6 mg, 2%)¹H NMR (400 MHz, dimethylsulfoxide-d₆) δ 14.01-12.27 (bs, 1H), 8.78 (d, J 2.4 Hz, 1H), 8.63 (s, 1H), 8.01 (dd, J 8.2, 2.5 Hz, 1H), 7.96 (d, J 7.5 Hz, 1H), 7.81-7.58 (m, 3H), 7.27 (t, J 7.6 Hz, 1H), 7.13 (d, J 7.6 Hz, 2H), 6.55 (d, J 5.3 Hz, 1H), 6.44 (s, 1H), 4.10-4.01 (m, 1H), 2.01 (s, 6H), 1.77-1.65 (m, 1H), 1.56-1.45 (m, 1H), 1.36 (s, 9H), 1.20-1.08 (m, 1H), 0.78 (d, J 6.6 Hz, 3H), 0.23 (d, J 6.3 Hz, 3H) ESI-MS m/z calc. 627.2879, found 628.6 (M+1)⁺; Retention time: 1.87 minutes; LC method A.

### Example 2: Preparation of Compound 2

### Step 1: tert-Butyl N-[(1R)-1-(hydroxymethyl)-3-methyl-butyl]-N-methyl-carbamate

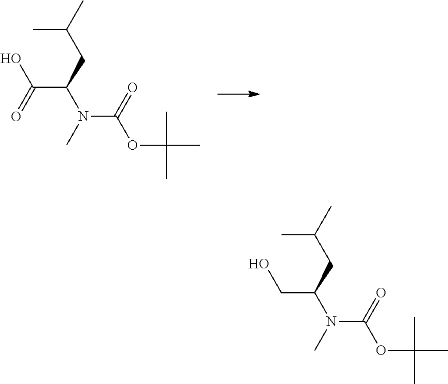 [SMILES: CC(C)C[C@H](C(=O)O)N(C)C(=O)OC(C)(C)C ; CC(C)C[C@H](CO)N(C)C(=O)OC(C)(C)C]

A solution of (2R)-2-[tert-butoxycarbonyl(methyl)amino]-4-methyl-pentanoic acid (0.44 g, 1.794 mmol) and borane-tetrahydrofuran (5.4 mL of 1 M in THF, 5.400 mmol) in THE (9 mL) was stirred for 16 h. The reaction was quenched with 1 M citric acid and extracted with ethyl acetate. The combined extracts were washed with water, dried over sodium sulfate, and evaporated under vacuum. The residue was purified by silica gel column chromatography with 0-5% methanol in dichloromethane to give a colorless oil, tert-butyl N-[(1R)-1-(hydroxymethyl)-3-methyl-butyl]-N-methyl-carbamate (0.37 g, 89%); ESI-MS m/z calc. 231.18344, found 232.2 (M+1)⁺; Retention time: 0.58 minutes; LC method D.

### Step 2: (2R)-4-Methyl-2-(methylamino)pentan-1-ol

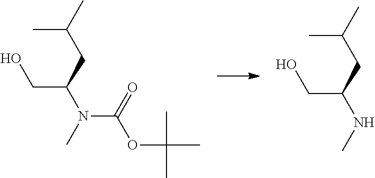

A solution of tert-butyl N-[(1R)-1-(hydroxymethyl)-3-methyl-butyl]-N-methyl-carbamate (0.37 g, 1.599 mmol) in HCl (5 mL of 4 M in dioxane, 20.00 mmol) was stirred for 15 h, and the solvent was evaporated under vacuum. The solids were triturated with diethyl ether and dried under vacuum to give a colorless solid, (2R)-4-methyl-2-(methylamino)pentan-1-ol (hydrochloride salt) (0.26 g, 97%) ESI-MS m/z calc. 131.13101, found 132.1 (M+1)⁺; Retention time: 0.25 minutes; LC method D.

### Step 3: (2R)-2-[Benzyl(methyl)amino]-4-methyl-pentan-1-ol

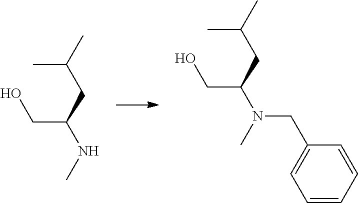

A solution of (2R)-4-methyl-2-(methylamino)pentan-1-ol (hydrochloride salt) (2.0 g, 11.93 mmol), benzyl bromide (1.4 mL, 11.77 mmol), and potassium carbonate (5.0 g, 36.18 mmol) in ethanol (45 mL) and water (15 mL) was stirred for 16 h. The reaction was diluted with water and extracted with ethyl acetate. The combined extracts were washed with water, dried over sodium sulfate, and evaporated under vacuum. The residue was purified by silica gel column chromatography with 0-5% methanol in dichloromethane to give a colorless oil, (2R)-2-[benzyl(methyl)amino]-4-methyl-pentan-1-ol (2.23 g, 84%); ¹H NMR (400 MHz, Chloroform-d) δ 7.36-7.22 (m, 5H), 3.70 (d, J 13.0 Hz, 1H), 3.53 (dd, J 10.5, 5.0 Hz, 1H), 3.47 (d, J 13.0 Hz, 1H), 3.33 (t, J 10.5 Hz, 1H), 2.86 (dddd, J 10.5, 9.0, 5.0, 3.8 Hz, 1H), 2.15 (s, 3H), 1.61-1.47 (m, 1H), 1.42 (ddd, J 13.1, 9.1, 3.8 Hz, 1H), 1.07 (ddd, J 13.5, 9.4, 5.0 Hz, 1H), 0.94 (d, J 6.5 Hz, 3H), 0.90 (d, J 6.5 Hz, 3H). ESI-MS m/z calc. 221.17796, found 222.4 (M+1)⁺; Retention time: 0.42 minutes; LC method D.

### Step 4: (2R)-2-[Benzyl(methyl)amino]-4-methyl-pentanal

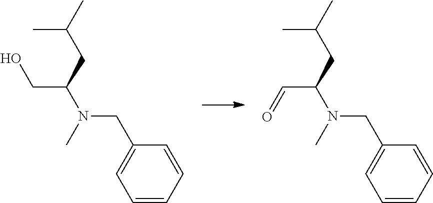 [SMILES: CC(C)C[C@H](CO)N(C)Cc1ccccc1 ; CC(C)C[C@H](C=O)N(C)Cc1ccccc1]

A solution of oxalyl chloride (0.95 mL, 10.89 mmol) in dichloromethane (6 mL) was cooled in a dry ice/acetone bath, and a solution of DMSO (0.90 mL, 12.68 mmol) in dichloromethane (2 mL) was slowly added. After 15 min, a solution of (2R)-2-[benzyl(methyl)amino]-4-methyl-pentan-1-ol (0.40 g, 1.807 mmol) in dichloromethane (3 mL) was slowly added, and the reaction was stirred for 1 h. DIPEA (3.8 mL, 21.82 mmol) was added, and after 45 min, the cooling bath was removed. After 20 min, the reaction was quenched with saturated aqueous ammonium chloride, separated, and the aqueous layer was further extracted with dichloromethane. The combined extracts were washed with water, dried over sodium sulfate, and evaporated under vacuum to give 1.0 g crude (2R)-2-[benzyl(methyl)amino]-4-methyl-pentanal, which was taken on directly to the next step without purification.

### Step 5: (4S,5R)-5-[Benzyl(methyl)amino]-2,7-dimethyl-octan-4-ol

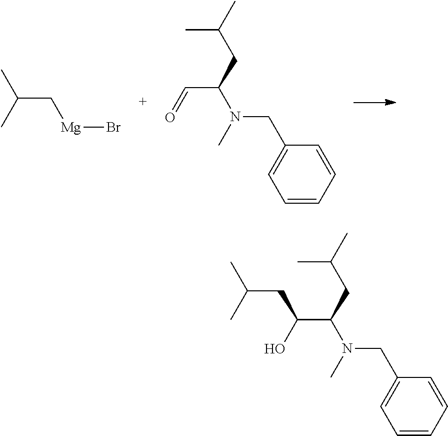

A solution of crude (2R)-2-[benzyl(methyl)amino]-4-methyl-pentanal (0.4 g, 1.824 mmol) (assumed amount in crude starting material) in THE (9 mL) was cooled in an ice bath, and isobutyl magnesium bromide (2.8 mL of 2 M in diethyl ether, 5.600 mmol) was slowly added. After 5 min, the cooling bath was removed, and the reaction was stirred for 2 h. The reaction was quenched with saturated aqueous ammonium chloride, diluted with water, and extracted with ethyl acetate. The combined extracts were washed with water, dried over sodium sulfate, and evaporated under vacuum. The residue was purified by silica gel column chromatography with 0-5% methanol in dichloromethane to give 0.3 g of a mixture containing product. The mixture was re-purified by silica gel column chromatography with 0-50% ethyl acetate in hexanes to give a colorless oil, (4S,5R)-5-[benzyl(methyl)amino]-2,7-dimethyl-octan-4-ol (0.15 g, 30%); ¹H NMR (400 MHz, Chloroform-d) δ 7.34-7.28 (m, 4H), 7.28-7.20 (m, 1H), 3.74 (ddd, J 10.2, 4.5, 2.7 Hz, 1H), 3.72-3.59 (m, 2H), 2.76 (td, J 7.1, 4.5 Hz, 1H), 2.24 (s, 3H), 1.83 (dpd, J 9.3, 6.7, 4.4 Hz, 1H), 1.70 (dq, J 13.3, 6.7 Hz, 1H), 1.54 (dt, J 14.1, 7.1 Hz, 1H), 1.35 (ddd, J 13.6, 10.2, 4.4 Hz, 1H), 1.30-1.19 (m, 2H), 0.95 (dd, J 6.3, 3.8 Hz, 6H), 0.92 (t, J 6.3 Hz, 6H). ESI-MS m/z calc. 277.24057, found 278.3 (M+1)⁺; Retention time: 0.51 minutes; LC method D.

### Step 6: 3-[[4-[(1S,2R)-2-[Benzyl(methyl)amino]-1-isobutyl-4-methyl-pentoxy]-6-(2,6-dimethylphenyl)pyrimidin-2-yl]sulfamoyl]benzoic acid

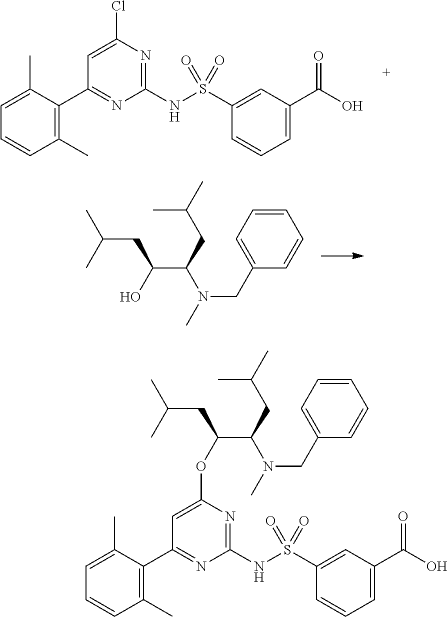 [SMILES: Cc1cccc(C)c1-c1cc(Cl)nc(NS(=O)(=O)c2cccc(C(=O)O)c2)n1 ; CC(C)C[C@H]([C@@H](O)CC(C)C)N(C)Cc1ccccc1 ; Cc1cccc(C)c1-c1cc(O[C@@H](CC(C)C)[C@@H](CC(C)C)N(C)Cc2ccccc2)nc(NS(=O)(=O)c2cccc(C(=O)O)c2)n1]

A solution of (4S,5R)-5-[benzyl(methyl)amino]-2,7-dimethyl-octan-4-ol (0.15 g, 0.5406 mmol), 3-[[4-chloro-6-(2,6-dimethylphenyl)pyrimidin-2-yl]sulfamoyl]benzoic acid (0.23 g, 0.5504 mmol), and sodium t-butoxide (0.21 g, 2.185 mmol) in THE (2.5 mL) was stirred for 15 h. Then, the reaction was stirred at 40° C. for 18 h. The reaction was quenched with 1 M citric acid, diluted with water, and extracted with ethyl acetate. The combined extracts were washed with brine, dried over sodium sulfate, and evaporated under vacuum. The residue was purified by silica gel column chromatography with 0-10% methanol in dichloromethane to give a colorless solid, 3-[[4-[(1S,2R)-2-[benzyl(methyl)amino]-1-isobutyl-4-methyl-pentoxy]-6-(2,6-dimethylphenyl)pyrimidin-2-yl]sulfamoyl]benzoic acid (41 mg, 12%) ESI-MS m/z calc. 658.3189, found 659.6 (M+1)⁺; Retention time: 0.65 minutes; LC method D.

### Step 7: 3-[[4-(2,6-Dimethylphenyl)-6-[(1S,2R)-1-isobutyl-4-methyl-2-(methylamino)pentoxy]pyrimidin-2-yl]sulfamoyl]benzoic acid

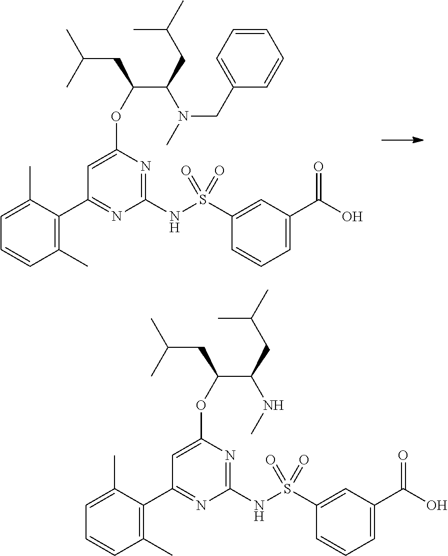

A mixture of 3-[[4-[(1S,2R)-2-[benzyl(methyl)amino]-1-isobutyl-4-methyl-pentoxy]-6-(2,6-dimethylphenyl)pyrimidin-2-yl]sulfamoyl]benzoic acid (41 mg, 0.06223 mmol) and dihydroxypalladium (10% w/w, 10 mg, 0.07121 mmol) in methanol (2 mL) was stirred under a hydrogen atmosphere for four hours. The reaction was filtered, and the solids were washed with methanol. The combined solutions were evaporated under vacuum to give a colorless solid, 3-[[4-(2,6-dimethylphenyl)-6-[(1S,2R)-1-isobutyl-4-methyl-2-(methylamino)pentoxy]pyrimidin-2-yl]sulfamoyl]benzoic acid (32 mg, 90%); ESI-MS m/z calc. 568.2719, found 569.5 (M+1)⁺; Retention time: 0.59 minutes; LC method D.

### Step 8: (10S,11R)-6-(2,6-Dimethylphenyl)-12-methyl-10,11-bis(2-methylpropyl)-9-oxa-2λ⁶-thia-3,5,12,19-tetraazatricyclo[12.3.1.14,8]nonadeca-1(18),4(19),5,7,14,16-hexaene-2,2,13-trione (Compound 2)

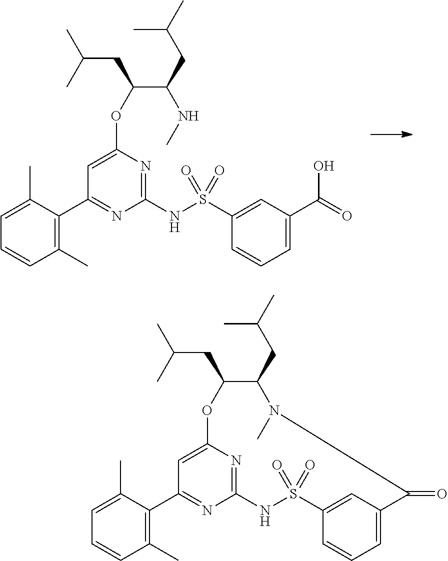

A solution of 3-[[4-(2,6-dimethylphenyl)-6-[(1S,2R)-1-isobutyl-4-methyl-2-(methylamino)pentoxy]pyrimidin-2-yl]sulfamoyl]benzoic acid (32 mg, 0.05627 mmol), HATU (26 mg, 0.06838 mmol), and triethylamine (24 μL, 0.1722 mmol) in DMF (3 mL) was stirred for 15 h. The reaction was concentrated and purified by reverse-phase preparative HPLC (C₁₈) to give a colorless solid, (10S,11R)-6-(2,6-dimethylphenyl)-12-methyl-10,11-bis(2-methylpropyl)-9-oxa-2λ⁶-thia-3,5,12,19-tetraazatricyclo[12.3.1.14,8]nonadeca-1(18),4(19),5,7,14,16-hexaene-2,2,13-trione (14.7 mg, 47%); ESI-MS m/z calc. 550.26135, found 551.4 (M+1)⁺; Retention time: 1.91 minutes; LC method A.

### Example 3: Preparation of Compound 3

### Step 1: Spiro[3.3]heptan-2-ylmethanol

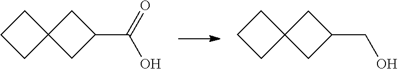 [SMILES: O=C(O)C1CC2(CCC2)C1 ; OCC1CC2(CCC2)C1]

To a solution of spiro[3.3]heptane-2-carboxylic acid (9.5 g, 67.770 mmol) in tetrahydrofuran (190 mL) cooled in an ice bath, was added dropwise Lithium aluminum hydride (in THF) (82 mL of 1 M, 82.000 mmol) over 15 minutes, maintaining an internal temperature<5° C. After the addition was complete, the reaction was stirred at 0-5° C. for 1 hour and at room temperature for 2 hours. The resulting mixture was cooled in an ice bath and water (10 mL) was added dropwise. Aqueous sodium hydroxide (15% w/w, 10 mL) was then added followed by additional water (25 mL). The reaction mixture was stirred for 15 minutes at room temperature and then it was filtered and rinsed with THF. The filtrate was concentrated in vacuo and the residue was diluted in EtOAc (100 mL) and washed with brine (20 mL). The organic phase was concentrated in vacuo to afford spiro[3.3]heptan-2-ylmethanol (8.4 g, 93%) as a light yellow oil. ¹H NMR (400 MHz, CDCl₃) δ 3.55 (d, J 6.8 Hz, 2H), 2.39-2.28 (m, 1H), 2.11-2.03 (m, 2H), 2.03-1.96 (m, 2H), 1.93-1.86 (m, 2H), 1.83-1.76 (m, 2H), 1.74-1.66 (m, 2H), 1.48-1.37 (m, 1H).

### Step 2: Spiro[3.3]heptane-2-carbaldehyde

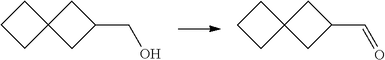 [SMILES: OCC1CC2(CCC2)C1 ; O=CC1CC2(CCC2)C1]

To a solution of spiro[3.3]heptan-2-ylmethanol (7.9 g, 59.471 mmol) in dichloromethane (160 mL) was added sodium bicarbonate (29 g, 345.21 mmol) and Dess-Martin periodinane (31 g, 73.089 mmol). The reaction mixture was stirred at room temperature 3 h. A 5% aqueous solution of sodium bicarbonate (200 mL) was added (strong evolution of gas) followed by a 10% w/w aqueous solution of Na₂S₂O₃ (200 mL). The mixture was vigorously stirred at room temperature for 3 h (until organic phase was clear). The phases were separated and the aqueous layer was extracted with DCM (2×250 mL). The combined organic layers were washed with a 10% w/w aqueous solution of Na₂S₂O₃ (200 mL), dried over magnesium sulfate, filtered and concentrated under reduced pressure to afford crude spiro[3.3]heptane-2-carbaldehyde (8.1 g, 99%) as a clear oil. ¹H NMR (400 MHz, CDCl₃) δ 9.70 (d, J 2.2 Hz, 1H), 3.07-2.97 (m, 1H), 2.27-2.13 (m, 4H), 2.07-2.01 (m, 2H), 1.94-1.88 (m, 2H), 1.85-1.77 (m, 2H).

### Step 3: methyl (Z)-2-(tert-butoxycarbonylamino)-3-spiro[3.3]heptan-2-yl-prop-2-enoate and methyl (E)-2-(tert-butoxycarbonylamino)-3-spiro[3.3]heptan-2-yl-prop-2-enoate

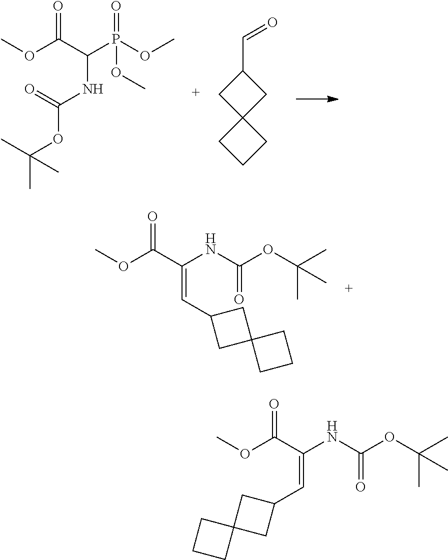

To a stirred solution of methyl 2-(tert-butoxycarbonylamino)-2-dimethoxyphosphoryl-acetate (1.3 g, 4.3735 mmol) and DBU (712.60 mg, 0.7 mL, 4.6809 mmol) in dichloromethane (10 mL) was added spiro[3.3]heptane-2-carbaldehyde (500 mg, 4.0264 mmol). The reaction mixture was stirred at room temperature for 16 h. Aqueous HCl (1 N) (10 mL) was added and the phases were separated. The aqueous layer was washed with DCM (2×20 mL). The combined organic layers were dried over magnesium sulfate, filtered and concentrated under reduced pressure. The crude residue was purified by chromatography on a silica gel cartridge (25 g+40 g) using a gradient of 0 to 30% EtOAc in heptanes to afford methyl (Z)-2-(tert-butoxycarbonylamino)-3-spiro[3.3]heptan-2-yl-prop-2-enoate (1.07 g, 90%) as a clear oil. ¹H NMR (400 MHz, CDCl₃) δ 6.58 (d, J 8.3 Hz, 1H), 5.85 (br. s., 1H), 3.80-3.73 (m, 3H), 3.15-3.04 (m, 1H), 2.33-2.23 (m, 2H), 2.06-2.01 (m, 2H), 1.94-1.75 (m, 6H), 1.51-1.42 (m, 9H). ESI-MS m/z calc. 295.1784, found 240.2 (M-55)⁺; Retention time: 1.98 minutes and methyl (E)-2-(tert-butoxycarbonylamino)-3-spiro[3.3]heptan-2-yl-prop-2-enoate (82 mg, 6%) as a yellow oil. ¹H NMR (400 MHz, CDCl₃) δ 6.80-6.78 (m, 1H), 6.53 (br. s., 1H), 3.81 (s, 3H), 3.69-3.59 (m, 1H), 2.34-2.25 (m, 2H), 2.05 (t, J 7.1 Hz, 2H), 1.92-1.76 (m, 6H), 1.47 (m, 9H). ESI-MS m/z calc. 295.1784, found 240.2 (M-55)⁺; Retention time: 2.05 minutes. LC method X.

### Step 4: Methyl (2R)-2-(tert-butoxycarbonylamino)-3-spiro[3.3]heptan-2-yl-propanoate

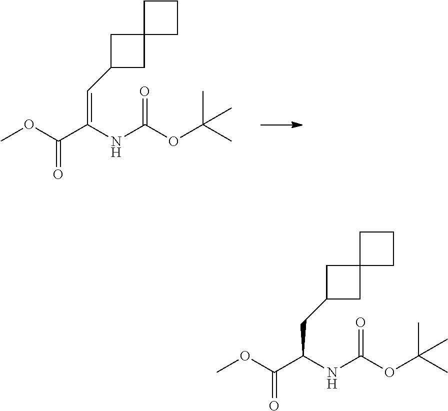 [SMILES: COC(=O)/C(=C/C1CC2(CCC2)C1)NC(=O)OC(C)(C)C ; COC(=O)[C@@H](CC1CC2(CCC2)C1)NC(=O)OC(C)(C)C]

Methyl (Z)-2-(tert-butoxycarbonylamino)-3-spiro[3.3]heptan-2-yl-prop-2-enoate (12.9 g, 42.363 mmol) was dissolved in ethanol (185 mL) and dioxane (60 mL). Nitrogen was passed through for about 10 min using a cannula. The solution was placed into an ultrasound bath (about 5 min), and 1,2-bis[(2R,5R)-2,5-diethylphospholano]benzene(1,5-cyclooctadiene)rhodium(I) trifluoromethanesulfonate (500 mg, 0.6781 mmol) was added. The mixture was hydrogenated under 3.5 bar hydrogen pressure and at room temperature for 24 hours. The reaction mixture was filtered through silica gel and the eluate was concentrated. The crude was directly purified by silica-gel column chromatography on a 100 g and 120 g column, eluting from 0 to 30% of ethyl acetate in heptanes to afford methyl (2R)-2-(tert-butoxycarbonylamino)-3-spiro[3.3]heptan-2-yl-propanoate (12.5 g, 99%) as a clear oil. ¹H NMR (400 MHz, CDCl₃) δ 4.93 (d, J 7.6 Hz, 1H), 4.26-4.15 (m, 1H), 3.72 (s, 3H), 2.23-2.05 (m, 3H), 1.98 (t, J 6.8 Hz, 2H), 1.88-1.65 (m, 6H), 1.64-1.54 (m, 2H), 1.44 (s, 9H). ESI-MS m/z calc. 297.194, found 198.2 (M-99)⁺; Retention time: 2.03 minutes, LC method X.

### Step 5: tert-Butyl N-[(1R)-1-(hydroxymethyl)-2-spiro[3.3]heptan-2-yl-ethyl]carbamate

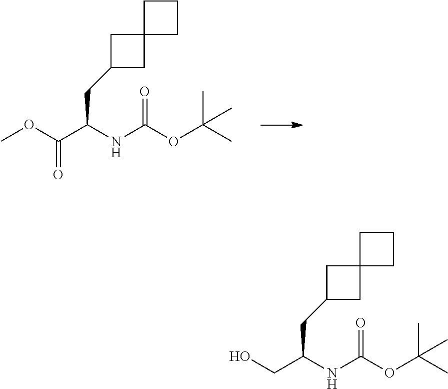 [SMILES: COC(=O)[C@@H](CC1CC2(CCC2)C1)NC(=O)OC(C)(C)C ; CC(C)(C)OC(=O)N[C@@H](CO)CC1CC2(CCC2)C1]

To a solution of methyl (2R)-2-(tert-butoxycarbonylamino)-3-spiro[3.3]heptan-2-yl-propanoate (12.5 g, 42.032 mmol) in tetrahydrofuran (125 mL) was added LiBH₄ (in THF) (55 mL of 2 M, 110.00 mmol) (no exotherm observed). The reaction mixture was stirred at room temperature for 3 hours. The reaction mixture was then poured slowly over a saturated aqueous solution of ammonium chloride (150 mL) at 0° C. (strong evolution of gas, but no exotherm). The product was extracted with EtOAc (3×150 mL). The combined organic layers were washed with brine (200 mL), dried over anhydrous sodium sulfate, filtered and concentrated under reduced pressure. The crude was purified through flash column chromatography (100 g+120 g) using a gradient of 0 to 50% EtOAc in heptanes to afford tert-butyl N-[(1R)-1-(hydroxymethyl)-2-spiro[3.3]heptan-2-yl-ethyl]carbamate (11 g, 97%) as a clear oil. ¹H NMR (400 MHz, CDCl₃) δ 4.58 (br. s., 1H), 3.66-3.43 (m, 3H), 2.22-2.06 (m, 3H), 2.06-2.03 (m, 1H), 1.99 (t, J 6.8 Hz, 2H), 1.88-1.72 (m, 4H), 1.65-1.48 (m, 4H), 1.45 (s, 9H). ESI-MS m/z calc. 269.1991, found 214.2 (M-55)⁺; Retention time: 1.87 minutes, LC method X.

### Step 6: (2R)-2-Amino-3-spiro[3.3]heptan-2-yl-propan-1-ol

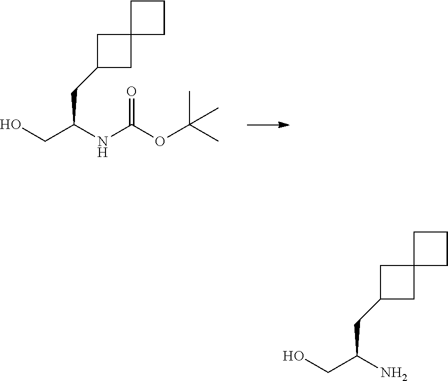 [SMILES: CC(C)(C)OC(=O)N[C@@H](CO)CC1CC2(CCC2)C1 ; N[C@@H](CO)CC1CC2(CCC2)C1]

To a solution of tert-butyl N-[(1R)-1-(hydroxymethyl)-2-spiro[3.3]heptan-2-yl-ethyl]carbamate (11 g, 40.835 mmol) in 1,4-dioxane (110 mL) was added hydrogen chloride (4 N in 1,4-dioxane) (110 mL of 4 M, 440.00 mmol). The reaction mixture was stirred at room temperature for 16 hours. The mixture was evaporated to give (2R)-2-amino-3-spiro[3.3]heptan-2-yl-propan-1-ol (hydrochloride salt) (7.8 g, 88%)(2R)-2-amino-3-spiro[3.3]heptan-2-yl-propan-1-ol (hydrochloride salt) (7.8 g, 88%) as a white solid. ¹H NMR (400 MHz, DMSO-d₆) δ 8.04 (br. s., 3H), 5.26 (br. s., 1H), 3.58-3.48 (m, 1H), 3.42-3.34 (m, 1H), 2.87 (br. s., 1H), 2.25-2.14 (m, 1H), 2.14-2.03 (m, 2H), 1.95 (t, J 7.2 Hz, 2H), 1.87-1.79 (m, 2H), 1.78-1.69 (m, 2H), 1.63-1.49 (m, 4H). ESI-MS m/z calc. 169.1467, found 170.2 (M+1)⁺; Retention time: 1.91 minutes, LC method Y.

### Step 7: 5-Morpholinopyridine-2-carbaldehyde

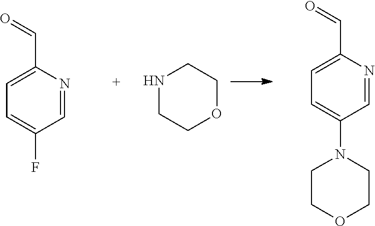 [SMILES: O=Cc1ccc(F)cn1 ; C1COCCN1 ; O=Cc1ccc(N2CCOCC2)cn1]

5-Fluoropyridine-2-carbaldehyde (5 g, 39.97 mmol) was combined with potassium carbonate (22.1 g, 159.9 mmol) and morpholine (7 mL, 80.27 mmol) in DMF (50 mL), and the reaction mixture was heated to 110° C. until completion. After cooling to room temperature, the reaction was diluted with methanol, filtered, and purified. A small quantity of water was added to the filtrate, which was then concentrated under reduced pressure. The resulting crude material was purified by chromatography on silica gel, eluting with a 0-10% gradient of methanol in DCM, to give 5-morpholinopyridine-2-carbaldehyde (6.389 g, 83%) as a light brown solid. ESI-MS m/z calc. 192.08987, found 193.2 (M+1)⁺; Retention time: 0.27 minutes, LC method D.

### Step 8: 3-[[4-[(2R)-2-Amino-3-spiro[3.3]heptan-2-yl-propoxy]-6-(2,6-dimethylphenyl)pyrimidin-2-yl]sulfamoyl]benzoic acid

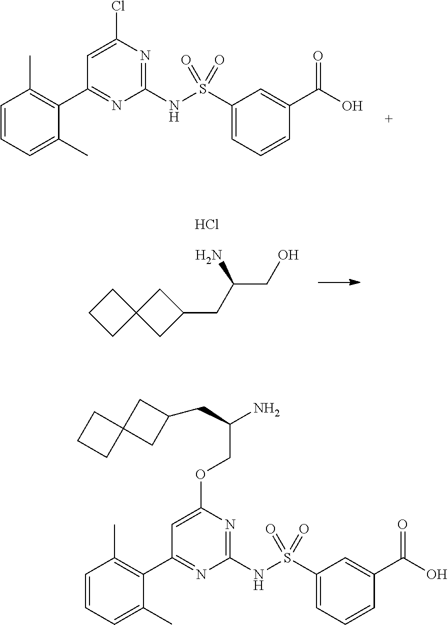 [SMILES: Cc1cccc(C)c1-c1cc(Cl)nc(NS(=O)(=O)c2cccc(C(=O)O)c2)n1 ; Cl ; N[C@@H](CO)CC1CC2(CCC2)C1 ; Cc1cccc(C)c1-c1cc(OC[C@H](N)CC2CC3(CCC3)C2)nc(NS(=O)(=O)c2cccc(C(=O)O)c2)n1]

3-[[4-Chloro-6-(2,6-dimethylphenyl)pyrimidin-2-yl]sulfamoyl]benzoic acid (1 g, 2.393 mmol) and (2R)-2-amino-3-spiro[3.3]heptan-2-yl-propan-1-ol (hydrochloride salt) (590 mg, 2.868 mmol) were combined in THE (5 mL) and stirred at room temperature for 5 minutes in a screwcap vial. Sodium tert-butoxide (1.35 g, 14.05 mmol) was then added in one portion. The reaction became warm and it was stirred for an additional 45 minutes without external heating. The reaction mixture was then partitioned between 40 mL 1M HCl and 40 mL ethyl acetate. The layers were separated, and the aqueous layer was extracted an additional 3× with ethyl acetate. The combined organics were washed with brine, dried over sodium sulfate, and concentrated to give as a white solid, 3-[[4-[(2R)-2-amino-3-spiro[3.3]heptan-2-yl-propoxy]-6-(2,6-dimethylphenyl)pyrimidin-2-yl]sulfamoyl]benzoic acid (hydrochloride salt) (1.455 g, 98%)/ESI-MS m/z calc. 550.225, found 551.5 (M+1)⁺; Retention time: 0.52 minutes, LC method D. Step 9: (11R)-6-(2,6-Dimethylphenyl)-12-[(5-morpholino-2-pyridyl)methyl]-2,2-dioxo-11-(spiro[3.3]heptan-2-ylmethyl)-9-oxa-2λ⁶-thia-3,5,12,19-tetrazatricyclo[12.3.1.14,8]nonadeca-1(18),4(19),5,7,14,16-hexaen-13-one (Compound 3)

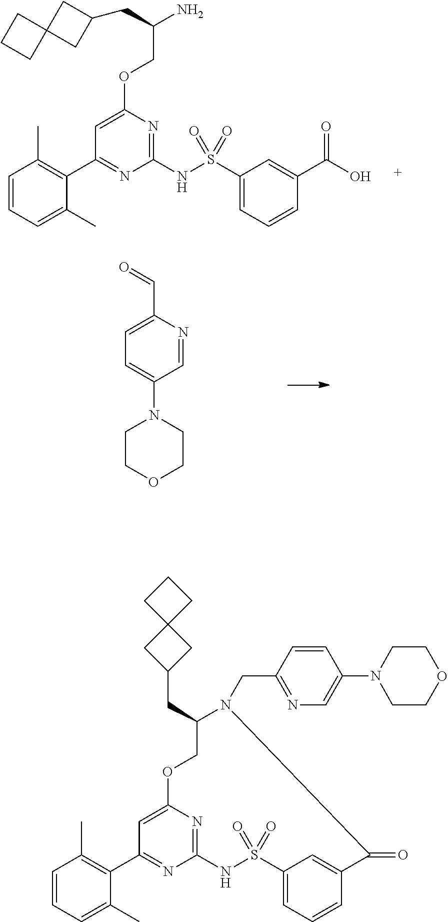 [SMILES: Cc1cccc(C)c1-c1cc(OC[C@H](N)CC2CC3(CCC3)C2)nc(NS(=O)(=O)c2cccc(C(=O)O)c2)n1 ; O=Cc1ccc(N2CCOCC2)cn1 ; Cc1cccc(C)c1-c1cc2nc(n1)NS(=O)(=O)c1cccc(c1)C(=O)N(Cc1ccc(N3CCOCC3)cn1)[C@H](CC1CC3(CCC3)C1)CO2]

3-[[4-[(2R)-2-Amino-3-spiro[3.3]heptan-2-yl-propoxy]-6-(2,6-dimethylphenyl)pyrimidin-2-yl]sulfamoyl]benzoic acid (hydrochloride salt) (50 mg, 0.08090 mmol) was combined with 5-morpholinopyridine-2-carbaldehyde (approximately 18.66 mg, 0.09708 mmol) in DCM (134.8 μL) and stirred at room temperature for 15 minutes. Sodium triacetoxyborohydride (approximately 17.15 mg, 0.08090 mmol) (1 equivalent) was added, followed by additional sodium triacetoxyborohydride (approximately 51.44 mg, 0.2427 mmol) (3 equivalents) 20 minutes later. The reaction was stirred at room temperature for an additional 30 minutes. It was then quenched with several drops of 1M HCl, diluted with 1:1 DMSO/methanol, filtered, and purified by reverse phase HPLC (1-70% ACN in water, HCl modifier, 15 min run) to give the reductive amination product. This product was dissolved in DMF (1.5 mL) and cooled to 0° C. N-Methylmorpholine (approximately 49.10 mg, 53.37 μL, 0.4854 mmol) was added, followed by CDMT (approximately 18.47 mg, 0.1052 mmol). After 30 minutes the reaction mixture was warmed to room temperature and it was stirred for an additional 2 hours at room temperature. The reaction mixture was then filtered and purified by reverse phase HPLC (1-70% ACN in water, HCl modifier). Pure fractions were dried to give (11R)-6-(2,6-dimethylphenyl)-12-[(5-morpholino-2-pyridyl)methyl]-2,2-dioxo-11-(spiro[3.3]heptan-2-ylmethyl)-9-oxa-2λ⁶-thia-3,5,12,19-tetrazatricyclo[12.3.1.14,8]nonadeca-1(18),4(19),5,7,14,16-hexaen-13-one (hydrochloride salt) (7 mg, 11%). ESI-MS m/z calc. 708.3094, found 709.6 (M+1)⁺; Retention time: 1.49 minutes; LC method A.

### Example 4: Preparation of Compound 4

### Step 1: tert-Butyl N-(2-hydroxyethyl)-N-isobutyl-carbamate

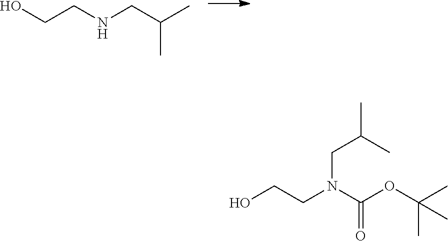

2-(Isobutylamino)ethanol (hydrochloride salt) (100 mg, 0.6508 mmol), Boc anhydride (148 mg, 0.6781 mmol), and cesium carbonate (231 mg, 0.7090 mmol) were combined in THE (2 mL) and stirred for 3 h. The reaction was partitioned between ethyl acetate and a 1M HCl solution. The organics were separated, washed with brine, dried over sodium sulfate and evaporated to give tert-butyl N-(2-hydroxyethyl)-N-isobutyl-carbamate (117 mg, 83%)¹H NMR (400 MHz, Chloroform-d) δ 3.75 (t, J 5.2 Hz, 2H), 3.40 (s, 2H), 3.05 (d, J 7.2 Hz, 2H), 1.87 (s, 1H), 1.46 (s, 9H), 0.89 (d, J=6.7 Hz, 6H).

### Step 2: 3-[[4-(2,6-Dimethylphenyl)-6-[2-(isobutylamino)ethoxy]pyrimidin-2-yl]sulfamoyl]benzoic acid

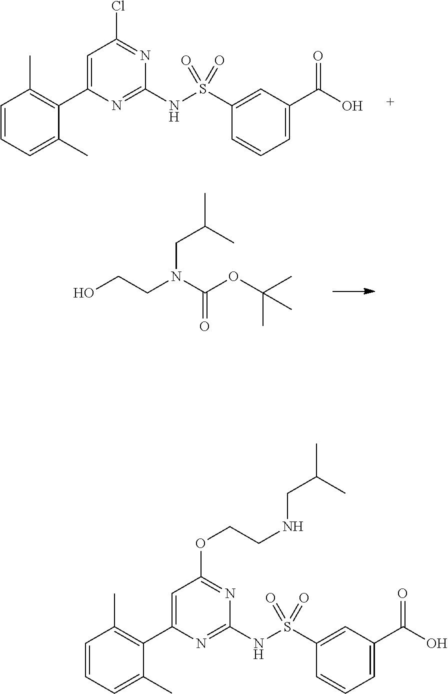 [SMILES: Cc1cccc(C)c1-c1cc(Cl)nc(NS(=O)(=O)c2cccc(C(=O)O)c2)n1 ; CC(C)CN(CCO)C(=O)OC(C)(C)C ; Cc1cccc(C)c1-c1cc(OCCNCC(C)C)nc(NS(=O)(=O)c2cccc(C(=O)O)c2)n1]

3-[[4-Chloro-6-(2,6-dimethylphenyl)pyrimidin-2-yl]sulfamoyl]benzoic acid (110 mg, 0.2632 mmol), tert-butyl N-(2-hydroxyethyl)-N-isobutyl-carbamate (117 mg, 0.5384 mmol), and sodium tert-butoxide (132 mg, 1.374 mmol) were dissolved in THE (1 mL) and stirred at room temperature for 16 h. The reaction was partitioned between ethyl acetate and a 1M HCl solution. The organics were separated, washed with brine, dried over sodium sulfate and evaporated. The crude material was purified by silica gel chromatography eluting with 0-100% ethyl acetate in hexanes to give 3-[[4-[2-[tert-butoxycarbonyl(isobutyl)amino]ethoxy]-6-(2,6-dimethylphenyl)pyrimidin-2-yl]sulfamoyl]benzoic acid (110 mg, 68%). This product was dissolved in 4M HCl in dioxane (1 mL of 4 M, 4.000 mmol) and stirred for 20 min. The reaction was evaporated and further dried to give 3-[[4-(2,6-dimethylphenyl)-6-[2-(isobutylamino)ethoxy]pyrimidin-2-yl]sulfamoyl]benzoic acid (hydrochloride salt) (98 mg, 68%) ESI-MS m/z calc. 498.1937, found 499.4 (M+1)⁺; Retention time: 0.4 minutes, LC method D.

### Step 3: 6-(2,6-Dimethylphenyl)-12-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 4)

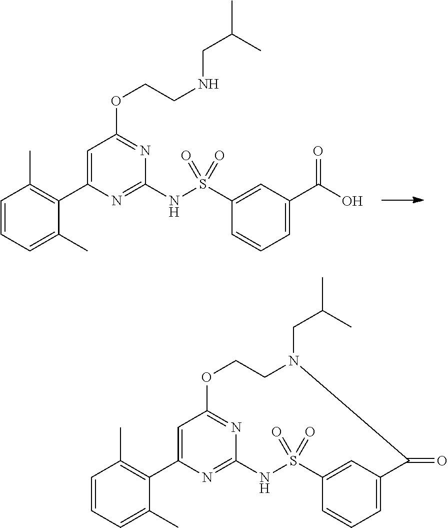 [SMILES: Cc1cccc(C)c1-c1cc(OCCNCC(C)C)nc(NS(=O)(=O)c2cccc(C(=O)O)c2)n1 ; Cc1cccc(C)c1-c1cc2nc(n1)NS(=O)(=O)c1cccc(c1)C(=O)N(CC(C)C)CCO2]

3-[[4-(2,6-Dimethylphenyl)-6-[2-(isobutylamino)ethoxy]pyrimidin-2-yl]sulfamoyl]benzoic acid (hydrochloride salt) (49 mg, 0.09158 mmol), HATU (36 mg, 0.09468 mmol), and triethylamine (50 μL, 0.3587 mmol) were combined in DMF (1 mL) and stirred for 30 min. The reaction mixture was filtered and purified by reverse phase HPLC utilizing a gradient of 1-99% acetonitrile in 5 mM aqueous HCl to yield 6-(2,6-dimethylphenyl)-12-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (21.3 mg, 48%) ESI-MS m/z calc. 480.18314, found 481.4 (M+1)⁺; Retention time: 1.56 minutes (LC method A).

### Example 5: Preparation of Compound 5

### Step 1: 3-[[4-Chloro-6-(2,6-dimethylphenyl)pyrimidin-2-yl]sulfamoyl]-N-(3-hydroxypropyl)-N-(2-pyridylmethyl)benzamide

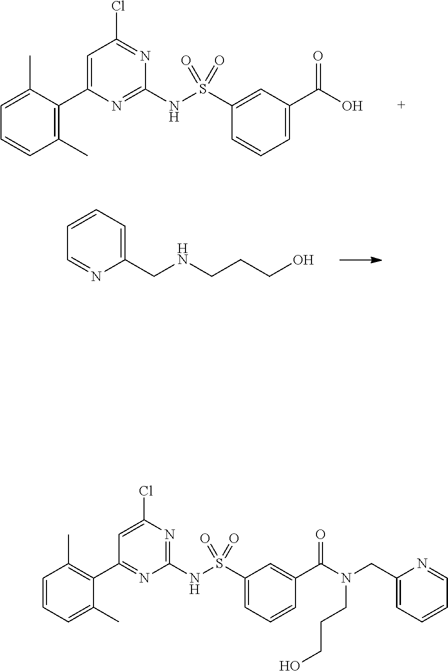 [SMILES: Cc1cccc(C)c1-c1cc(Cl)nc(NS(=O)(=O)c2cccc(C(=O)O)c2)n1 ; OCCCNCc1ccccn1 ; Cc1cccc(C)c1-c1cc(Cl)nc(NS(=O)(=O)c2cccc(C(=O)N(CCCO)Cc3ccccn3)c2)n1]

3-[[4-Chloro-6-(2,6-dimethylphenyl)pyrimidin-2-yl]sulfamoyl]benzoic acid (60 mg, 0.1436 mmol) was dissolved in dichloromethane and added to N,N-diisopropylcarbodiimide. The mixture was allowed to stir at room temperature for 30 minutes. The obtained suspension was then added to 3-(2-pyridylmethylamino)propan-1-ol (approximately 26.26 mg, 0.1580 mmol). Solid sodium bicarbonate was added last. The reaction mixture was allowed to stir overnight at room temperature. The product was isolated by UV-triggered reverse-phase HPLC 9method using a Luna C₁₈ (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C.) to yield 3-[[4-chloro-6-(2,6-dimethylphenyl)pyrimidin-2-yl]sulfamoyl]-N-(3-hydroxypropyl)-N-(2-pyridylmethyl)benzamide (16.1 mg, 20%).ESI-MS m/z calc. 565.155, found 566.3 (M+1)⁺; Retention time: 1.3 minutes; LC method A.

### Step 2: 6-(2,6-Dimethylphenyl)-2,2-dioxo-13-(2-pyridylmethyl)-9-oxa-2λ⁶-thia-3,5,13,20-tetrazatricyclo[13.3.1.14,8]icosa-1(19),4(20),5,7,15,17-hexaen-14-one (Compound 5)

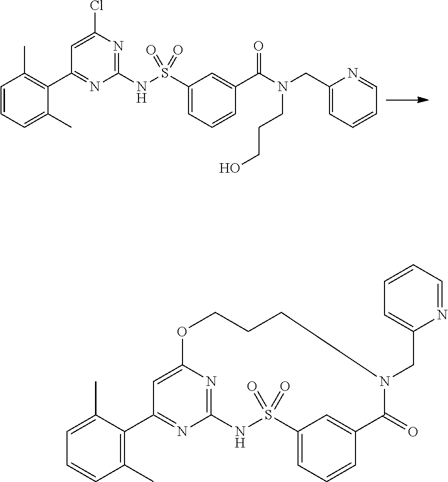 [SMILES: Cc1cccc(C)c1-c1cc(Cl)nc(NS(=O)(=O)c2cccc(C(=O)N(CCCO)Cc3ccccn3)c2)n1 ; Cc1cccc(C)c1-c1cc2nc(n1)NS(=O)(=O)c1cccc(c1)C(=O)N(Cc1ccccn1)CCCO2]

A solution of 3-[[4-chloro-6-(2,6-dimethylphenyl)pyrimidin-2-yl]sulfamoyl]-N-(3-hydroxypropyl)-N-(2-pyridylmethyl)benzamide (16.1 mg, 0.02844 mmol) in 1-methyl-pyrrolidin-2-one (5 mL) was added to sodium hydride (approximately 11.37 mg, 12.63 μL, 0.2844 mmol) (60 wt % dispersion in mineral oil). The reaction mixture was sealed and allowed to stir at 70° C. for 2 hours. Samples were purified using a reverse phase HPLC method using a Luna C₁₈ (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. 6-(2,6-Dimethylphenyl)-2,2-dioxo-13-(2-pyridylmethyl)-9-oxa-2λ⁶-thia-3,5,13,20-tetrazatricyclo[13.3.1.14,8]icosa-1(19),4(20),5,7,15,17-hexaen-14-one (3.5 mg, 23%) was obtained. ESI-MS m/z calc. 529.17834, found 530.3 (M+1)⁺; Retention time: 1.22 minutes; LC method A.

### Example 7: Preparation of Compound 6

### Step 1: 5-Fluoropyrimidine-2-carbaldehyde

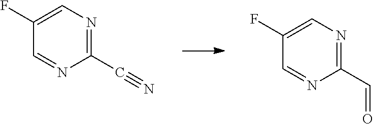 [SMILES: N#Cc1ncc(F)cn1 ; O=Cc1ncc(F)cn1]

Into a solution of 5-fluoropyrimidine-2-carbonitrile (10 g, 77.993 mmol) in anhydrous THE (200 mL) was added 1.0 M DIBAL-H in toluene (117 mL of 1 M, 117.00 mmol) at −78° C. dropwise for 30 minutes. After the addition, the reaction was stirred for another 2 hours at the same temperature. Methanol (40 mL) was added to the reaction mixture at −78° C. The reaction temperature was slowly raised to rt, and then it was diluted with 10% HCl (aqueous) (60 mL) and concentrated HCl (20 mL) (pH=3). Solid NaCl was added to saturate the aqueous layer. The reaction mixture was stirred for 1 hours until both layers were clear. Two layers were separated. The aqueous layer was extracted with DCM (10×100 mL). The combined organic layers were dried over anhydrous sodium sulfate and concentrated under vacuum. The residual toluene solution was directly loaded onto a silica gel column and purified using 0 to 60% diethyl ether in DCM. The desired fractions were combined and concentrated under vacuum to furnish 5-fluoropyrimidine-2-carbaldehyde (5.545 g, 54%) as a yellow liquid. ESI-MS m/z calc. 126.0229, found 127.2 (M+1)⁺; Retention time: 0.34 minutes. ¹H NMR (500 MHz, DMSO-d₆) δ 9.96 (s, 1H), 9.17 (d, J 0.8 Hz, 2H).LC method W.

### Step 2: 5-Morpholinopyrimidine-2-carbaldehyde

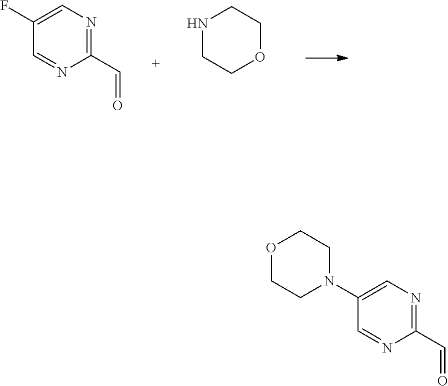 [SMILES: O=Cc1ncc(F)cn1 ; C1COCCN1 ; O=Cc1ncc(N2CCOCC2)cn1]

Into a solution of 5-fluoropyrimidine-2-carbaldehyde (1.29 g, 6.6194 mmol) in anhydrous DMF (10 mL) was added morpholine (1.1988 g, 1.2 mL, 13.760 mmol) and potassium carbonate (3.65 g, 26.410 mmol). The reaction was stirred at 110° C. for 4 hours. After centrifuge, the DMF solution was directly subjected to HPLC purification using 0 to 40% ACN in water (buffered with 0.1% HCl). The desired fractions were combined and lyophilized to furnish 5-morpholinopyrimidine-2-carbaldehyde (hydrochloride salt) (1.4515 g, 91%) as a yellow solid. ESI-MS m/z calc. 193.0851, found 194.3 (M+1)⁺; Retention time: 1.27 minutes. ¹H NMR (500 MHz, DMSO-d₆) δ 9.79 (s, 1H), 8.65 (s, 2H), 3.82-3.66 (m, 4H), 3.54-3.37 (m, 4H). LC method W.

### Step 3: 2-Bromo-1-isopropyl-3-methyl-benzene

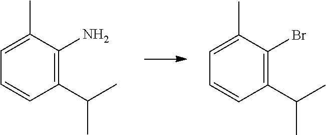

Into a solution of 2-isopropyl-6-methyl-aniline (23.750 g, 25 mL, 159.15 mmol) in concentrated HBr (240 mL) and water (240 mL) was added a solution of sodium nitrite (13.18 g, 191.03 mmol) in water (100 mL) at 0° C. After the addition, the reaction was stirred at 0° C. for 20 minutes. In a separate flask was charged with a solution of Cu(I)Br (22.9 g, 159.64 mmol) in concentrated HBr (240 mL) and heated to 60° C. The diazonium salt solution was added dropwise to the reaction mixture. After the addition was finished, the reaction was stirred at the same temperature for 1 hour, and then it was cooled down to rt. The solution was extracted with diethyl ether (3×175 mL). The combined ether layers were washed with saturated Sodium bicarbonate (200 mL) and brine (150 mL), dried over anhydrous sodium sulfate and concentrated under vacuum. The residue was directly loaded onto a silica gel column and purified using 0 to 40% diethyl ether in hexane (the product comes out very early) to furnish 2-bromo-1-isopropyl-3-methyl-benzene (19.2 g, 54%) as a clear oil. ¹H NMR (250 MHz, CDCl₃) δ 7.23-7.03 (m, 3H), 3.48 (m, 1H), 2.44 (d, J 0.6 Hz, 3H), 1.26 (d, J 0.5 Hz, 3H), 1.24 (d, J 0.5 Hz, 3H).

### Step 4: (2-Isopropyl-6-methyl-phenyl)boronic acid

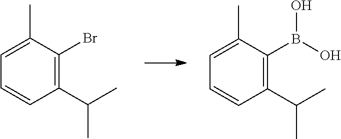

To a solution of 2-bromo-1-isopropyl-3-methyl-benzene (14.77 g, 69.306 mmol) in anhydrous THE (400 mL) at −78° C. was added dropwise n-BuLi in hexanes (33 mL of 2.5 M, 82.500 mmol). The solution was stirred at this temperature for 15 min before adding trimethyl borate (21.436 g, 23 mL, 206.29 mmol) dropwise at the same temperature. After the addition the solution was allowed to warm up to 0° C. and stir for 1 hour. The solution was then quenched with 1M HCl and stirred for 3 hours and then partitioned with EtOAc. The aqueous was extracted with EtOAc (2×20 mL). The organic layers were then combined, washed with brine, dried over anhydrous sodium sulfate, filtered, and concentrated under reduced pressure. The crude was purified via silica gel column chromatography (eluting 0-40% Ether in hexanes) to yield (2-isopropyl-6-methyl-phenyl)boronic acid (4.91 g, 40%) as a white solid. ¹H NMR (500 MHz, DMSO-d₆) δ 8.12 (d, J 5.2 Hz, 2H), 7.13 (m, 1H), 7.03 (d, J 7.8 Hz, 1H), 6.92 (d, J 7.4 Hz, 1H), 2.82 (m, 1H), 2.26 (d, J 2.3 Hz, 3H), 1.18 (m, 6H).

### Step 5: tert-Butyl N-tert-butoxycarbonyl-N-[4-chloro-6-(2-isopropyl-6-methyl-phenyl)pyrimidin-2-yl]carbamate

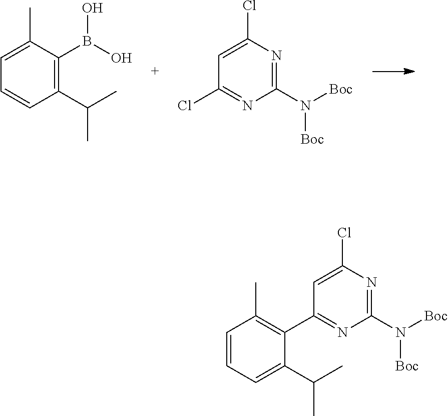 [SMILES: Cc1cccc(C(C)C)c1B(O)O ; CC(C)(C)OC(=O)N(C(=O)OC(C)(C)C)c1nc(Cl)cc(Cl)n1 ; Cc1cccc(C(C)C)c1-c1cc(Cl)nc(N(C(=O)OC(C)(C)C)C(=O)OC(C)(C)C)n1]

To a mixture of tert-butyl N-tert-butoxycarbonyl-N-(4,6-dichloropyrimidin-2-yl)carbamate (7.57 g, 20.784 mmol), (2-isopropyl-6-methyl-phenyl)boronic acid (3.17 g, 17.805 mmol), cesium carbonate (14.7 g, 45.117 mmol), and Pd(dppf)Cl₂ (1.47 g, 1.8001 mmol) was added a solvent mixture of DME (70 mL) and water (70 mL) that had been degassed for 30 minutes. During the addition of the solvents the reaction vial was being flushed with nitrogen. Once the addition was finished the vial was sealed and heated to 80° C. and the reaction mixture was stirred for 2 h. The mixture was then quenched with DI water (40 mL) and EtOAc (70 mL). The aqueous layer was extracted with EtOAc (2×50 mL). The organic layers were combined, washed with brine, dried over anhydrous sodium sulfate, filtered, and concentrated under reduced pressure. The crude was purified via silica gel column chromatography (eluting 0 to 30% EtOAc in hexanes) to yield tert-butyl N-tert-butoxycarbonyl-N-[4-chloro-6-(2-isopropyl-6-methyl-phenyl)pyrimidin-2-yl]carbamate (6.02 g, 59%) as a white solid. ESI-MS m/z calc. 461.2081, found 462.3 (M+1)⁺; Retention time: 4.04 minutes, LC method T.

### Step 6: 4-Dhloro-6-(2-isopropyl-6-methyl-phenyl)pyrimidin-2-amine

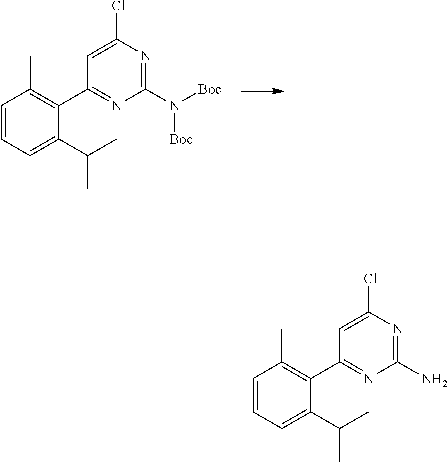 [SMILES: Cc1cccc(C(C)C)c1-c1cc(Cl)nc(N(C(=O)OC(C)(C)C)C(=O)OC(C)(C)C)n1 ; Cc1cccc(C(C)C)c1-c1cc(Cl)nc(N)n1]

To a solution of tert-butyl N-tert-butoxycarbonyl-N-[4-chloro-6-(2-isopropyl-6-methyl-phenyl)pyrimidin-2-yl]carbamate (6.02 g, 10.425 mmol) in anhydrous DCM (35 mL) at 0° C. was added HCl in dioxane (35 mL of 4 M, 140.00 mmol). The solution was then raised to RT and stirred for 2 hours. The solution was then quenched with sodium bicarbonate (75 mL) and partitioned with DCM (50 mL). The aqueous layer was extracted with DCM (2×50 mL). The organic layers were then combined, washed with brine, dried over anhydrous sodium sulfate, filtered, and concentrated under reduced pressure to yield 4-chloro-6-(2-isopropyl-6-methyl-phenyl)pyrimidin-2-amine (3.99 g, 117%) as a white solid. ESI-MS m/z calc. 261.1033, found 262.0 (M+1)⁺; Retention time: 3.0 minutes, LC method T.

### Step 7: Methyl 3-[[4-chloro-6-(2-isopropyl-6-methyl-phenyl)pyrimidin-2-yl]sulfamoyl]benzoate

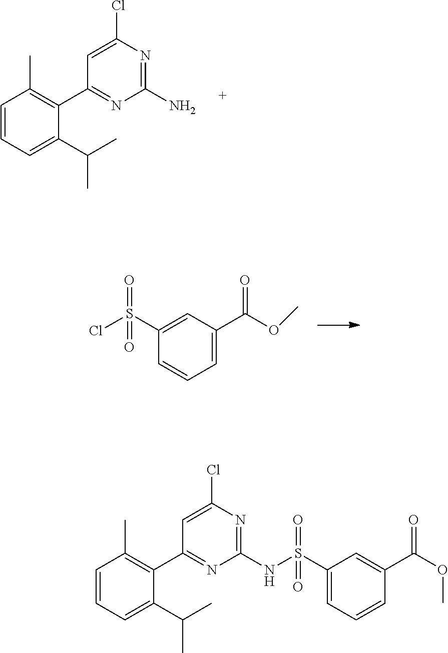 [SMILES: Cc1cccc(C(C)C)c1-c1cc(Cl)nc(N)n1 ; COC(=O)c1cccc(S(=O)(=O)Cl)c1 ; COC(=O)c1cccc(S(=O)(=O)Nc2nc(Cl)cc(-c3c(C)cccc3C(C)C)n2)c1]

To a solution of 4-chloro-6-(2-isopropyl-6-methyl-phenyl)pyrimidin-2-amine (3.99 g, 12.195 mmol) and methyl 3-chlorosulfonylbenzoate (4.27 g, 18.197 mmol) in anhydrous THE (40 mL) at 0° C. was added dropwise lithium tert-amoxide (in heptanes) (8.7600 g, 30 mL of 40% w/w, 37.244 mmol) and then stirred at this temperature for 5 minutes after addition. The solution was then warmed to RT and stirred for 2 hours. The reaction was then quenched with 1M HCl (50 mL) and partitioned with EtOAc (50 mL). The aqueous layer was extracted with EtOAc (2×50 mL). The organic layers were then combined, washed with brine, dried over anhydrous sodium sulfate, filtered, and concentrated under reduced pressure. The crude was purified via silica gel column chromatography (eluting 0 to 30% EtOAc in hexanes) to yield methyl 3-[[4-chloro-6-(2-isopropyl-6-methyl-phenyl)pyrimidin-2-yl]sulfamoyl]benzoate (4.39 g, 63%) as a white solid. ESI-MS m/z calc. 459.102, found 460.1 (M+1)⁺; Retention time: 3.44 minutes, LC method T.

### Step 8: 3-[[4-Chloro-6-(2-isopropyl-6-methyl-phenyl)pyrimidin-2-yl]sulfamoyl]benzoic acid

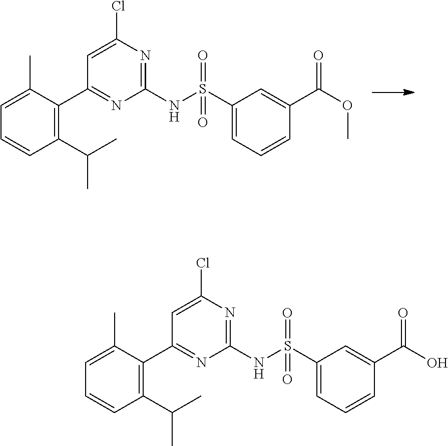 [SMILES: COC(=O)c1cccc(S(=O)(=O)Nc2nc(Cl)cc(-c3c(C)cccc3C(C)C)n2)c1 ; Cc1cccc(C(C)C)c1-c1cc(Cl)nc(NS(=O)(=O)c2cccc(C(=O)O)c2)n1]

To a solution of methyl 3-[[4-chloro-6-(2-isopropyl-6-methyl-phenyl)pyrimidin-2-yl]sulfamoyl]benzoate (4.39 g, 7.6357 mmol) in THE (25 mL) was added NaOH (40 mL of 1 M, 40.000 mmol). The reaction was stirred at RT for 4 hours. The reaction was then quenched with 1M HCl (50 mL) and partitioned with EtOAc (30 mL). The aqueous was then extracted with EtOAc (2×20 mL). The organic layers were then combined, washed with brine, dried over anhydrous sodium sulfate, filtered, and concentrated under reduced pressure. The crude was purified via reverse phase HPLC (gradient 45-90% acetonitrile in water buffered by 0.1% TFA) to yield 3-[[4-chloro-6-(2-isopropyl-6-methyl-phenyl)pyrimidin-2-yl]sulfamoyl]benzoic acid (2.38 g, 67%) as a white solid. ESI-MS m/z calc. 445.0863, found 446.2 (M+1)⁺; Retention time: 2.58 minutes. ¹H NMR (500 MHz, DMSO-d₆) δ 13.41 (s, 1H), 12.45 (s, 1H), 8.41 (t, J 1.8, 1.8 Hz, 1H), 8.15 (dt, J 7.8, 1.4, 1.4 Hz, 1H), 8.09 (dt, J 8.0, 1.4, 1.4 Hz, 1H), 7.64 (t, J 7.8, 7.8 Hz, 1H), 7.33-7.27 (m, 2H), 7.21 (d, J 7.8 Hz, 1H), 7.07 (d, J 7.5 Hz, 1H), 2.34 (p, J 6.8 Hz, 1H), 1.75 (s, 3H), 1.03 (d, J 6.8 Hz, 3H), 0.89 (d, J 6.8 Hz, 3H), LC method W.

### Step 9: 3-[[4-[(2R)-2-Amino-4,4-dimethyl-pentoxy]-6-(2-isopropyl-6-methyl-phenyl)pyrimidin-2-yl]sulfamoyl]benzoic acid

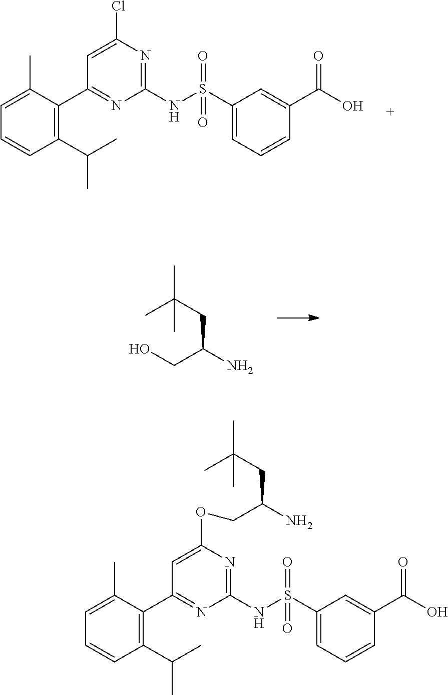

To a solution of 3-[[4-chloro-6-(2-isopropyl-6-methyl-phenyl)pyrimidin-2-yl]sulfamoyl]benzoic acid (1.38 g, 2.9586 mmol) and (2R)-2-amino-4,4-dimethyl-pentan-1-ol (hydrochloride salt) (462 mg, 2.7553 mmol) in anhydrous THE (25 mL) was added sodium tert-butoxide (1.48 g, 15.400 mmol). The reaction was stirred at RT for 2 hours. The crude was then quenched with 1M HCl (35 mL) and partitioned with EtOAc (35 mL). The aqueous layer was extracted with EtOAc (2×30 mL). The organic layers were combined, washed with brine, dried over anhydrous sodium sulfate, filtered, and concentrated under reduced pressure. The crude was purified via reverse phase HPLC (gradient 30-70% acetonitrile in water buffered by 5 mM HCl) to yield 3-[[4-[(2R)-2-amino-4,4-dimethyl-pentoxy]-6-(2-isopropyl-6-methyl-phenyl)pyrimidin-2-yl]sulfamoyl]benzoic acid (hydrochloride salt) (1.175 g, 66%) as a white solid. ESI-MS m/z calc. 540.2406, found 541.3 (M+1)⁺; Retention time: 1.96 minutes. ¹H NMR (500 MHz, DMSO-d₆) δ 8.44 (d, J 1.9 Hz, 1H), 8.11 (dd, J 16.2, 7.8 Hz, 4H), 7.67 (t, J 7.8, 7.8 Hz, 1H), 7.33 (s, 1H), 7.25 (d, J 7.8 Hz, 1H), 7.11 (d, J 7.5 Hz, 1H), 6.27 (s, 1H), 4.29 (dd, J 23.0, 11.7 Hz, 1H), 4.08 (s, 1H), 3.55 (s, 1H), 2.53 (s, 1H), 1.95 (s, 3H), 1.61-1.43 (m, 2H), 1.06 (d, J 7.0 Hz, 6H), 0.93 (s, 9H).LC method W.

### Step 10: (11R)-11-(2,2-Dimethylpropyl)-6-[2-methyl-6-(propan-2-yl)phenyl]-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 6)

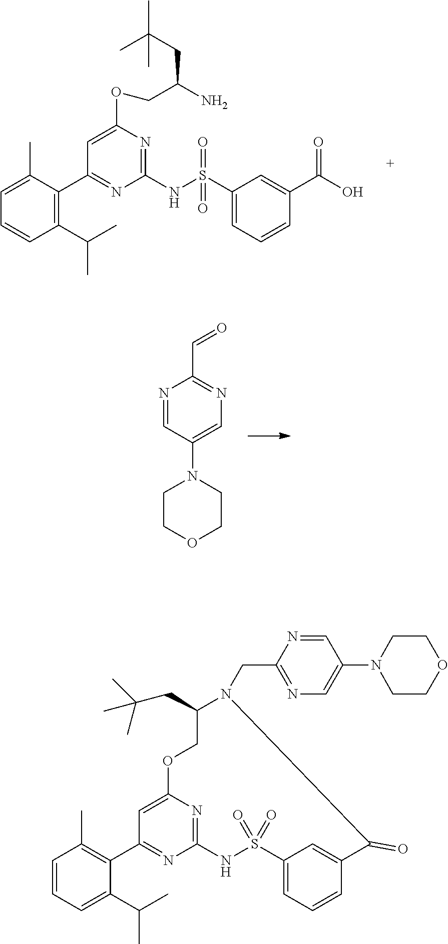 [SMILES: Cc1cccc(C(C)C)c1-c1cc(OC[C@H](N)CC(C)(C)C)nc(NS(=O)(=O)c2cccc(C(=O)O)c2)n1 ; O=Cc1ncc(N2CCOCC2)cn1 ; Cc1cccc(C(C)C)c1-c1cc2nc(n1)NS(=O)(=O)c1cccc(c1)C(=O)N(Cc1ncc(N3CCOCC3)cn1)[C@H](CC(C)(C)C)CO2]

A 4 mL vial was charged with 3-[[4-[(2R)-2-amino-4,4-dimethyl-pentoxy]-6-(2-isopropyl-6-methyl-phenyl)pyrimidin-2-yl]sulfamoyl]benzoic acid (hydrochloride salt) (53 mg, 0.09183 mmol), 5-morpholinopyrimidine-2-carbaldehyde (hydrochloride salt) (26 mg, 0.1132 mmol) and DCM (200 μL). The solution was stirred at room temperature for 15 min. sodium triacetoxyborohydride (25 mg, 0.1180 mmol) was added, the vial was purged with nitrogen, capped and the mixture was stirred at room temperature for 30 min. More sodium triacetoxyborohydride (67 mg, 0.3161 mmol) was added and the mixture was stirred at room temperature for 3.5 h. The solution was quenched with a minimum amount of 1N aqueous HCl. Methanol and DMSO were added. The solution was filtered and purified by reverse phase preparative HPLC (Cis column) using a gradient (1-99% over 30 min) of acetonitrile in aqueous 5 mM HCl to provide 3-[[4-[(2R)-4,4-dimethyl-2-[(5-morpholinopyrimidin-2-yl)methylamino]pentoxy]-6-(2-isopropyl-6-methyl-phenyl)pyrimidin-2-yl]sulfamoyl]benzoic acid (hydrochloride salt) (40.8 mg, 59%) as a tan solid. ESI-MS m/z calc. 717.3309, found 718.36 (M+1)⁺; Retention time: 1.39 minutes (LC method A).

The material was combined in a 4 mL vial with CDMT (15 mg, 0.08543 mmol) and anhydrous DMF (1 mL). The mixture was cooled down in an ice-water bath. 4-methylmorpholine (30 μL, 0.2729 mmol) was added and the mixture was stirred in the cooling bath that was allowed to warm to room temperature. After 2.5 days, the solution was diluted with DMSO, filtered and purified by reverse phase preparative HPLC (Cis column) using a gradient (1-99% over 30 min) of acetonitrile in aqueous 5 mM HCl to provide (11R)-11-(2,2-dimethylpropyl)-6-[2-methyl-6-(propan-2-yl)phenyl]-12-{[5-(morpholin-4-yl)pyrimidin-2-yl]methyl}-9-oxa-2λ⁶-thia-3,5,12,19-tetraazatricyclo[12.3.1.14,8]nonadeca-1(17),4(19),5,7,14(18),15-hexaene-2,2,13-trione (13.8 mg, 21%) as an off-white solid. ESI-MS m/z calc. 699.3203, found 700.39 (M+1)⁺; Retention time: 1.85 minutes (LC method A). ¹H NMR (400 MHz, DMSO-d₆) 1:1 mixture of atropisomers, δ 13.55-11.40 (broad m, 1H), 8.70 (s, 1H), 8.50 (s, 2H), 7.96 (br s, 1H), 7.67 (br s, 2H), 7.35 (t, J 7.8 Hz, 1H), 7.31-7.20 (m, 1H), 7.19-7.03 (m, 1H), 6.43 (br s, 1H), 5.38 (dd, J 10.7, 4.1 Hz, 1H), 4.84 (d, J 16.4 Hz, 1H), 4.61 (d, J 16.5 Hz, 1H), 4.27-3.98 (m, 2H), 3.75 (t, J 4.7 Hz, 4H), 3.23 (t, J 4.8 Hz, 4H), 2.66-2.59 (m, 0.5H), 2.25 (p, J=6.8 Hz, 0.5H), 2.11 (s, 1.5H), 1.85 (s, 1.5H), 1.82-1.72 (m, 1H), 1.38 (t, J 13.8 Hz, 1H), 1.17 (d, J 6.8 Hz, 1.5H), 1.10 (d, J 6.8 Hz, 1.5H), 1.06-0.94 (m, 3H), 0.56 (s, 4.5H), 0.55 (s, 4.5H).

### Example 8: Preparation of Compound 7

### Step 1: 1-Benzyloxy-2-bromo-3-methyl-benzene

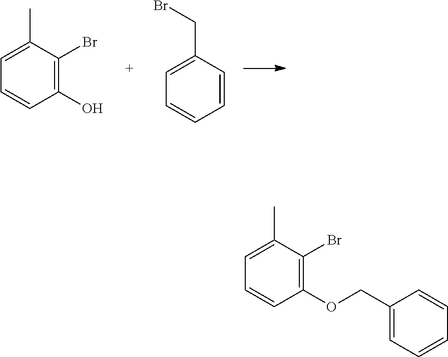

2-Bromo-3-methyl-phenol (20 g, 104.79 mmol) in DMF (100 mL) was added potassium carbonate (29.000 g, 209.83 mmol) and BnBr (27.322 g, 19 mL, 159.75 mmol). The reaction was allowed to proceed for 2 h at 60° C. The reaction mixture was filtered. The filtrate was added water (300 mL) and extracted with ethyl acetate (3×50 mL). The organic fractions were combined and dried over anhydrous sodium sulfate. Evaporation of the solvent gave the crude, which was purified by combi-flash (silica gel 330 g×2, 0-20% EtOAc in hexane) to afford 1-benzyloxy-2-bromo-3-methyl-benzene (25 g, 73%) as a colorless oil (retention time: 4.11 min, no ionization). ¹H NMR (500 MHz, Chloroform-d) δ 7.50 (dd, J 8.0, 1.2 Hz, 2H), 7.40 (td, J 6.8, 6.3, 1.5 Hz, 2H), 7.36-7.30 (m, 1H), 7.13 (t, J 7.9, 7.9 Hz, 1H), 6.88 (dd, J 7.4, 1.1 Hz, 1H), 6.79 (dd, J=8.2, 1.4 Hz, 1H), 5.16 (s, 2H), 2.45 (s, 3H).

### Step 2: 2-(2-Benzyloxy-6-methyl-phenyl)-4,4,5,5-tetramethyl-1,3,2-dioxaborolane &Br 0

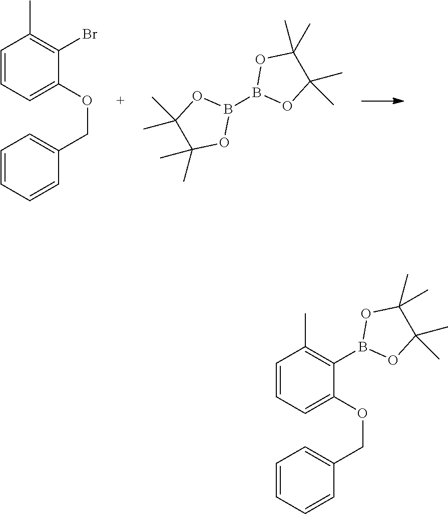

In a 250 mL seal tube was dissolved 1-benzyloxy-2-bromo-3-methyl-benzene (21 g, 71.981 mmol) in dioxane (200 mL) and to it was added KOAc (13 g, 132.46 mmol) and the mixture was degassed under vacuum for several minutes. Then 4,4,5,5-tetramethyl-2-(4,4,5,5-tetramethyl-1,3,2-dioxaborolan-2-yl)-1,3,2-dioxaborolane (26 g, 102.39 mmol) was added, followed by Pd(dppf)2Cl2 (4.8 g, 6.5600 mmol) and the reaction was degassed again, sealed and heated to 100° C. for 20 hours. The reaction was cooled to room temperature, saturated aqueous ammonium chloride (200 mL) was added and extracted with ethyl acetate (3×100 mL). The combined organic extracts were washed with brine, dried over anhydrous sodium sulfate, filtered and concentrated. The resulting brown oil was purified utilizing combi-flash (silica gel 330 g×2, dry loading, 0-20% EtOAc in Hex) to afford 2-(2-benzyloxy-6-methyl-phenyl)-4,4,5,5-tetramethyl-1,3,2-dioxaborolane (9.6 g, 39%) as a colorless oil. ESI-MS m/z calc. 324.1897, found 325.2 (M+1)⁺; Retention time: 4.01 minutes, LC method T.

### Step 3: tert-Butyl N-[4-(2-benzyloxy-6-methyl-phenyl)-6-chloro-pyrimidin-2-yl]-N-tert-butoxycarbonyl-carbamate

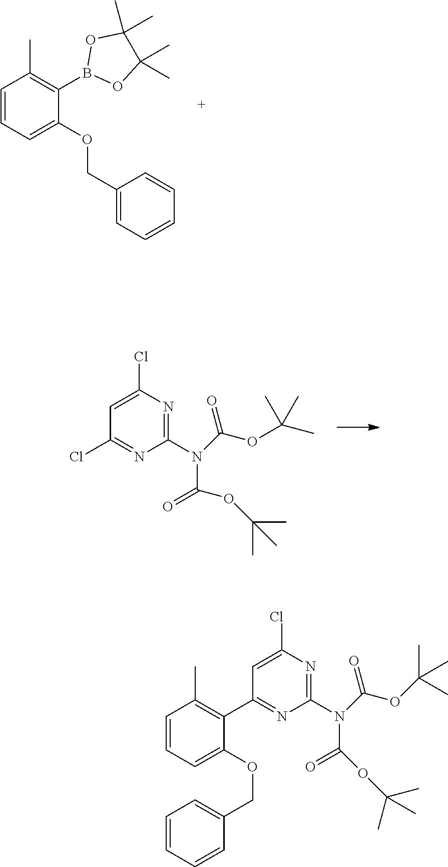 [SMILES: Cc1cccc(OCc2ccccc2)c1B1OC(C)(C)C(C)(C)O1 ; CC(C)(C)OC(=O)N(C(=O)OC(C)(C)C)c1nc(Cl)cc(Cl)n1 ; Cc1cccc(OCc2ccccc2)c1-c1cc(Cl)nc(N(C(=O)OC(C)(C)C)C(=O)OC(C)(C)C)n1]

To a slurry of tert-butyl N-tert-butoxycarbonyl-N-(4,6-dichloropyrimidin-2-yl)carbamate (5.6 g, 14.606 mmol),2-(2-benzyloxy-6-methyl-phenyl)-4,4,5,5-tetramethyl-1,3,2-dioxaborolane (5.6 g, 16.409 mmol) and cesium carbonate (11.7 g, 35.910 mmol) in dimethoxyethane (50 mL) and water (10 mL) was added Pd(dppf)Cl₂ (1 g, 1.3667 mmol) and the mixture vigorously stirred under nitrogen at 80° C. (reflux) for 2 hours. The reaction was cooled to ambient temperature and diluted with water (100 mL). The aqueous phase was separated and extracted with EtOAc (3×30 mL). The organic phase was washed with 100 mL of brine, dried over anhydrous sodium sulfate and concentrated. The crude was purified by combi-flash (silica gel 220 g, dry loading, 0-50% EtOAc in hexane) to afford tert-butyl N-[4-(2-benzyloxy-6-methyl-phenyl)-6-chloro-pyrimidin-2-yl]-N-tert-butoxycarbonyl-carbamate (4.5 g, 56%) as a white solid. ESI-MS m/z calc. 525.203, found 526.5 (M+1)⁺; Retention time: 4.46 minutes, LC method T.

### Step 4: 4-(2-Benzyloxy-6-methyl-phenyl)-6-chloro-pyrimidin-2-amine

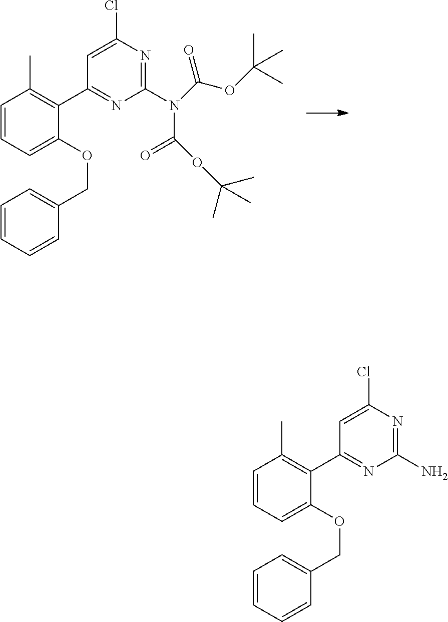 [SMILES: Cc1cccc(OCc2ccccc2)c1-c1cc(Cl)nc(N(C(=O)OC(C)(C)C)C(=O)OC(C)(C)C)n1 ; Cc1cccc(OCc2ccccc2)c1-c1cc(Cl)nc(N)n1]

tert-Butyl N-[4-(2-benzyloxy-6-methyl-phenyl)-6-chloro-pyrimidin-2-yl]-N-tert-butoxycarbonyl-carbamate (6.8 g, 12.281 mmol) in DCM (20 mL) was added HCl in dioxane (15 mL of 4 M, 60.000 mmol). The reaction mixture was stirred at rt for 12 h. After completion, the volatiles were removed under reduced pressure to afford 4-(2-benzyloxy-6-methyl-phenyl)-6-chloro-pyrimidin-2-amine (hydrochloride salt) (5.1 g, 92%) as a white solid. ESI-MS m/z calc. 325.0982, found 326.2 (M+1)⁺; Retention time: 3.27 minutes, LC method T.

### Step 5: Methyl 3-[[4-(2-benzyloxy-6-methyl-phenyl)-6-chloro-pyrimidin-2-yl]sulfamoyl]benzoate

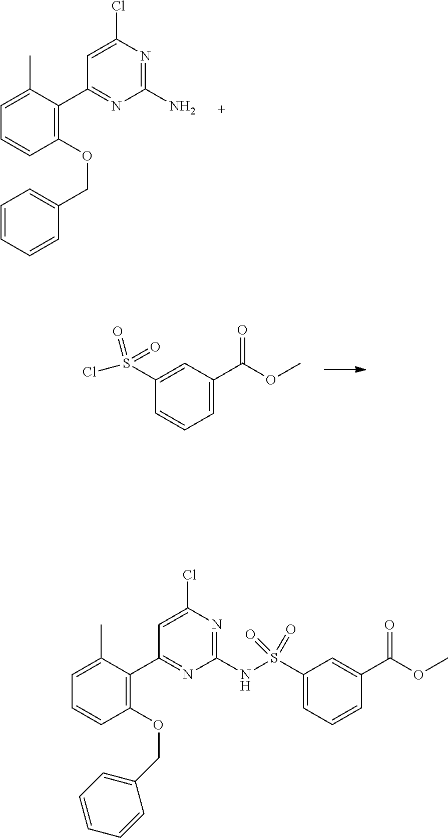 [SMILES: Cc1cccc(OCc2ccccc2)c1-c1cc(Cl)nc(N)n1 ; COC(=O)c1cccc(S(=O)(=O)Cl)c1 ; COC(=O)c1cccc(S(=O)(=O)Nc2nc(Cl)cc(-c3c(C)cccc3OCc3ccccc3)n2)c1]

4-(2-Benzyloxy-6-methyl-phenyl)-6-chloro-pyrimidin-2-amine (hydrochloride salt) (8.5 g, 21.118 mmol) was dissolved in THE (100 mL) and cooled in an ice bath under stirring and nitrogen To the cold solution, methyl 3-chlorosulfonylbenzoate (7.5 g, 31.962 mmol) in solution in THE (20 mL) was added. At 0° C., lithium tert-pentoxide in heptane (17 g, 23.288 mL of 40% w/w, 72.278 mmol) was added dropwise and the reaction was stirred at room temperature for 2 h. The reaction was quenched with HCl 1 N (50 mL). The reaction was diluted with water (50 ml). The aqueous phase was extracted with EtOAc (3×30 mL). The organic phase was dried over anhydrous sodium sulfate, filtered, and concentrated. The crude was purified by combi-flash (silica gel 220 g, dry loading in silica gel, 0-60% acetone in hexane) to give methyl 3-[[4-(2-benzyloxy-6-methyl-phenyl)-6-chloro-pyrimidin-2-yl]sulfamoyl]benzoate (9.9 g, 85%) as a white solid. ESI-MS m/z calc. 523.09686, found 524.3 (M+1)⁺; Retention time: 3.74 minutes; LC method T.

### Step 6: 3-[[4-(2-Benzyloxy-6-methyl-phenyl)-6-chloro-pyrimidin-2-yl]sulfamoyl]benzoic acid

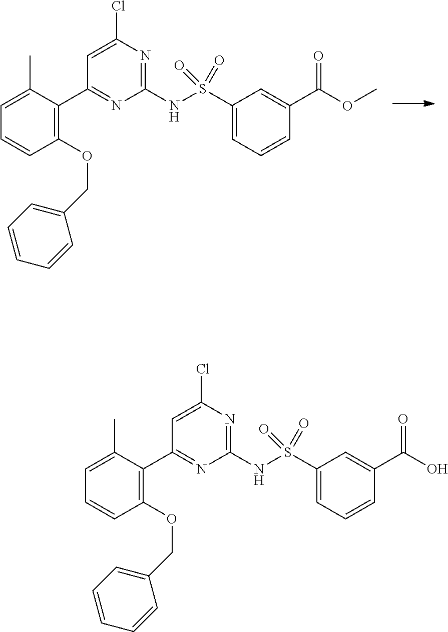 [SMILES: COC(=O)c1cccc(S(=O)(=O)Nc2nc(Cl)cc(-c3c(C)cccc3OCc3ccccc3)n2)c1 ; Cc1cccc(OCc2ccccc2)c1-c1cc(Cl)nc(NS(=O)(=O)c2cccc(C(=O)O)c2)n1]

Methyl 3-[[4-(2-benzyloxy-6-methyl-phenyl)-6-chloro-pyrimidin-2-yl]sulfamoyl]benzoate (3.7 g, 6.8494 mmol) in THE (40 mL) was added aqueous NaOH (20 mL of 3 M, 60.000 mmol). The reaction mixture was stirred at room temperature for 2 h. After completion, aqueous HCl (1 M) was added to acidify the solution. The aqueous phase was extracted with EtOAc (3×30 mL). The combined organic layers were dried over anhydrous sodium sulfate and concentrated under vacuo to afford 3-[[4-(2-benzyloxy-6-methyl-phenyl)-6-chloro-pyrimidin-2-yl]sulfamoyl]benzoic acid (3.35 g, 86%) as a white solid. ESI-MS m/z calc. 509.0812, found 510.0 (M+1)⁺; Retention time: 3.34 minutes, LC method T.

### Step 7: 3-[[4-[(2R)-2-Amino-4,4-dimethyl-pentoxy]-6-(2-benzyloxy-6-methyl-phenyl)pyrimidin-2-yl]sulfamoyl]benzoic acid

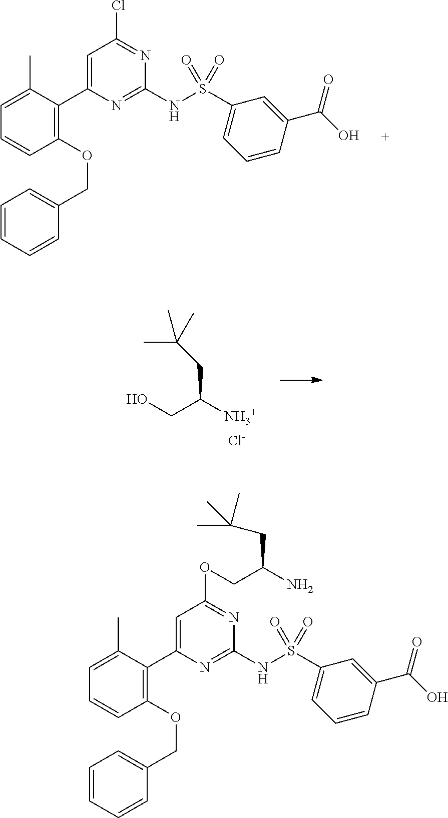

To a solution of 3-[[4-(2-benzyloxy-6-methyl-phenyl)-6-chloro-pyrimidin-2-yl]sulfamoyl]benzoic acid (3.35 g, 6.2407 mmol) and [(1R)-1-(hydroxymethyl)-3,3-dimethyl-butyl]ammonium chloride (1.2 g, 7.0135 mmol) in THE (50 mL) maintained at 5° C. with an ice-water bath was added NaOtBu (3 g, 31.216 mmol) and the mixture was stirred at room temperature for 3 h. The reaction was added 1N HCl and extracted with EtOAc. The organic layers were combined, dried and concentrated. The crude was purified by HPLC (Mobile phase A: 0.1% HCl in water; Mobile phase B: acetonitrile; Method: 25% B to 75% B, 60 mL/min) to afford 3-[[4-[(2R)-2-amino-4,4-dimethyl-pentoxy]-6-(2-benzyloxy-6-methyl-phenyl)pyrimidin-2-yl]sulfamoyl]benzoic acid (hydrochloride salt) (3 g, 73%) as a white solid. ESI-MS m/z calc. 604.2356, found 605.5 (M+1)⁺; Retention time: 2.54 minutes, LC method T.

### Step 8: 3-[[4-(2-Benzyloxy-6-methyl-phenyl)-6-[(2R)-4,4-dimethyl-2-[(5-morpholino-2-pyridyl)methylamino]pentoxy]pyrimidin-2-yl]sulfamoyl]benzoic acid

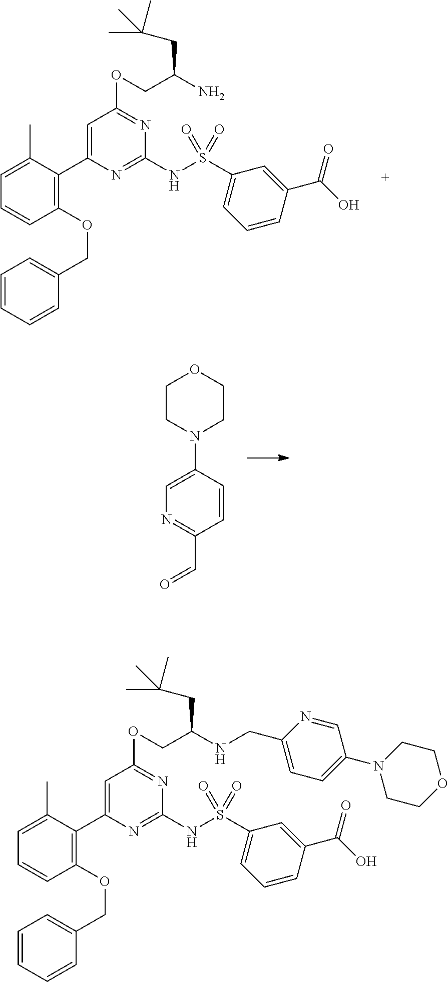 [SMILES: Cc1cccc(OCc2ccccc2)c1-c1cc(OC[C@H](N)CC(C)(C)C)nc(NS(=O)(=O)c2cccc(C(=O)O)c2)n1 ; O=Cc1ccc(N2CCOCC2)cn1 ; Cc1cccc(OCc2ccccc2)c1-c1cc(OC[C@@H](CC(C)(C)C)NCc2ccc(N3CCOCC3)cn2)nc(NS(=O)(=O)c2cccc(C(=O)O)c2)n1]

3-[[4-[(2R)-2-Amino-4,4-dimethyl-pentoxy]-6-(2-benzyloxy-6-methyl-phenyl)pyrimidin-2-yl]sulfamoyl]benzoic acid (hydrochloride salt) (3 g, 4.4450 mmol) was combined with 5-morpholinopyridine-2-carbaldehyde (1.2 g, 5.9309 mmol) in DCM (20 mL) and stirred at rt for 15 minutes. Sodium triacetoxyborohydride (1.9 g, 8.9648 mmol) was then added. The reaction was allowed to stir at rt for an additional 60 minutes, then was quenched with 1M HCl (20 mL), then was extracted with DCM (3×30 mL). The combined organic layers were dried over anhydrous sodium sulfate and concentrated under vacuum to afford 3-[[4-(2-benzyloxy-6-methyl-phenyl)-6-[(2R)-4,4-dimethyl-2-[(5-morpholino-2-pyridyl)methylamino]pentoxy]pyrimidin-2-yl]sulfamoyl]benzoic acid (hydrochloride salt) (3.95 g, 98%). ESI-MS m/z calc. 780.3305, found 781.3 (M+1)⁺; Retention time: 2.77 minutes, LC method T.

### Step 9: (11R)-6-(2-Benzyloxy-6-methyl-phenyl)-11-(2,2-dimethylpropyl)-12-[(5-morpholino-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 7)

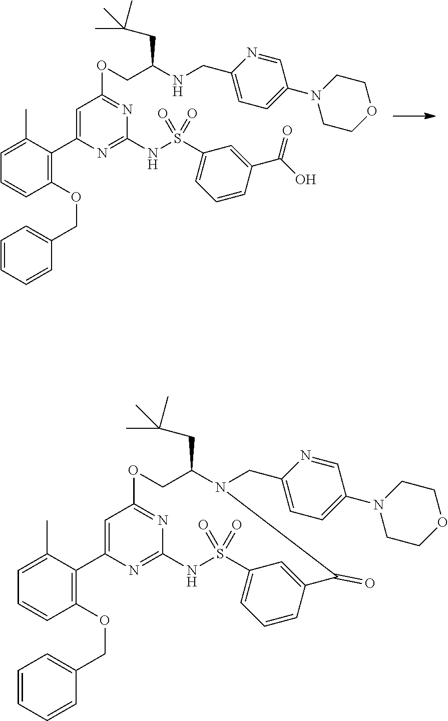 [SMILES: Cc1cccc(OCc2ccccc2)c1-c1cc(OC[C@@H](CC(C)(C)C)NCc2ccc(N3CCOCC3)cn2)nc(NS(=O)(=O)c2cccc(C(=O)O)c2)n1 ; Cc1cccc(OCc2ccccc2)c1-c1cc2nc(n1)NS(=O)(=O)c1cccc(c1)C(=O)N(Cc1ccc(N3CCOCC3)cn1)[C@H](CC(C)(C)C)CO2]

Into a solution of 3-[[4-(2-benzyloxy-6-methyl-phenyl)-6-[(2R)-4,4-dimethyl-2-[(5-morpholino-2-pyridyl)methylamino]pentoxy]pyrimidin-2-yl]sulfamoyl]benzoic acid (30 mg, 0.0376 mmol) in DCM (10 mL) at 0° C. was added HATU (15 mg, 0.0394 mmol), TBTU (10 mg, 0.0311 mmol), (4-(4,6-dimethoxy-1,3,5-triazin-2-yl)-4-methyl-morpholinium chloride) (9 mg, 0.0325 mmol) and DIPEA (37.100 mg, 0.05 mL, 0.2871 mmol). The reaction was stirred at rt for 1 h. The reaction was quenched with 10% citric acid aqueous solution (1 mL). The aqueous solution was extracted with ethyl acetate (3×5 mL). The combined organic layers were dried over anhydrous sodium sulfate and concentrated under vacuum. The reside was purified by HPLC (Mobile phase A: 0.1% HCl in water; Mobile phase B: acetonitrile; Method: 35% B to 75% B, 60 mL/min)) to furnish (11R)-6-(2-benzyloxy-6-methyl-phenyl)-11-(2,2-dimethylpropyl)-12-[(5-morpholino-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (7 mg, 22%) as a yellow solid. ESI-MS m/z calc. 762.32, found 763.6 (M+1)⁺; Retention time: 2.24 minutes, LC method T. ¹H NMR (500 MHz, DMSO-d₆) δ 8.56 (s, 1H), 8.30 (d, J 2.9 Hz, 1H), 7.94 (d, J 7.7 Hz, 1H), 7.78-7.58 (m, 4H), 7.33 (t, J 8.0, 8.0 Hz, 1H), 7.22 (d, J 19.0 Hz, 5H), 7.00 (d, J 8.4 Hz, 1H), 6.91 (d, J 7.7 Hz, 1H), 6.44 (s, 1H), 5.27 (dd, J 10.4, 4.3 Hz, 1H), 5.10-4.99 (m, 2H), 4.81 (d, J 15.5 Hz, 1H), 4.59 (d, J 15.7 Hz, 1H), 4.28 (t, J 11.2, 11.2 Hz, 1H), 4.02 (d, J=12.9 Hz, 1H), 3.74 (dd, J 6.1, 3.7 Hz, 4H), 3.25 (t, J 4.9, 4.9 Hz, 4H), 2.07 (s, 3H), 1.75 (dd, J=15.4, 9.1 Hz, 1H), 1.34 (d, J=15.2 Hz, 1H), 0.45 (s, 9H).

### Example 9: Preparation of Compound 8 and Compound 9

### Step 1: 1-(4-Bromophenyl)-2-(methylamino)ethanol

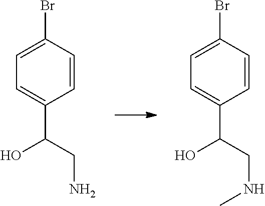

To a nitrogen sparged round bottom flask was added a solution of 2-amino−1-(4-bromophenyl)ethanol (1.33 g, 6.155 mmol) in ethyl formate (40 mL, 495.2 mmol). The reaction solution was heated to reflux under an atmosphere of nitrogen. After 12 h, the volatiles were removed in vacuo to afford the formamide as a colorless solid. To this material was added anhydrous THE (25 mL) and LAH (280 mg, 7.377 mmol) was added at 0° C. The reaction solution was allowed to warm to rt under stirring for 3 h. The reaction solution was quenched with addition of potassium sodium tartrate solution and EtOAc was added. The solution was stirred for 30 min prior to partitioning of layers. The EtOAc phase was dried over sodium sulfate, filtered and concentrated in vacuo to afford 1-(4-bromophenyl)-2-(methylamino)ethanol (740 mg, 52%). ¹H NMR (400 MHz, DMSO-d₆) δ 7.63-7.43 (m, 2H), 7.42-7.28 (m, 2H), 5.35 (s, 1H), 4.61 (t, J 6.2 Hz, 1H), 2.60-2.54 (m, 2H), 2.29 (s, 3H). ESI-MS m/z calc. 229.01022, found 230.2 (M+1)⁺; Retention time: 0.3 minutes, (LC method D).

### Step 2: 3-[[4-[1-(4-Bromophenyl)-2-[tert-butoxycarbonyl(methyl)amino]ethoxy]-6-(2,6-dimethylphenyl)pyrimidin-2-yl]sulfamoyl]benzoic acid

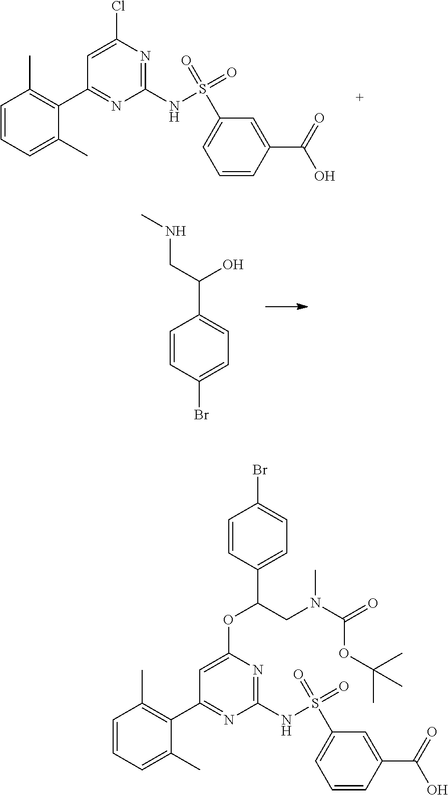 [SMILES: Cc1cccc(C)c1-c1cc(Cl)nc(NS(=O)(=O)c2cccc(C(=O)O)c2)n1 ; CNCC(O)c1ccc(Br)cc1 ; Cc1cccc(C)c1-c1cc(OC(CN(C)C(=O)OC(C)(C)C)c2ccc(Br)cc2)nc(NS(=O)(=O)c2cccc(C(=O)O)c2)n1]

To a nitrogen sparged round bottom flask was added 3-[[4-chloro-6-(2,6-dimethylphenyl)pyrimidin-2-yl]sulfamoyl]benzoic acid (895 mg, 2.142 mmol) and a solution of 1-(4-bromophenyl)-2-(methylamino)ethanol (740 mg, 3.216 mmol) in anhydrous THE (20 mL). To the stirred solution at 0° C. was added sodium tert-butoxide (722 mg, 7.513 mmol). The reaction solution was stirred at 0° C. for 30 min, then allowed to stir warming to room temperature for 30 min. To the reaction solution at room temp was added Boc anhydride (562 mg, 2.575 mmol). The reaction solution was allowed to stir at room temp overnight. The reaction mixture was diluted with EtOAc and quenched with aqueous saturated ammonium chloride. The organics phase was separated, washed with brine, dried over sodium sulfate and evaporated. The crude material was purified by flash chromatography on a 12 g silica gel column using a gradient of 100% DCM to 25% MeOH/DCM to afford purified fractions of 3-[[4-[1-(4-bromophenyl)-2-[tert-butoxycarbonyl(methyl)amino]ethoxy]-6-(2,6-dimethylphenyl)pyrimidin-2-yl]sulfamoyl]benzoic acid (659 mg, 43%). ESI-MS m/z calc. 710.141, found 711.2 (M+1)⁺; Retention time: 0.79 minutes; LC method D.

### Step 3: 10-(4-Bromophenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 8)

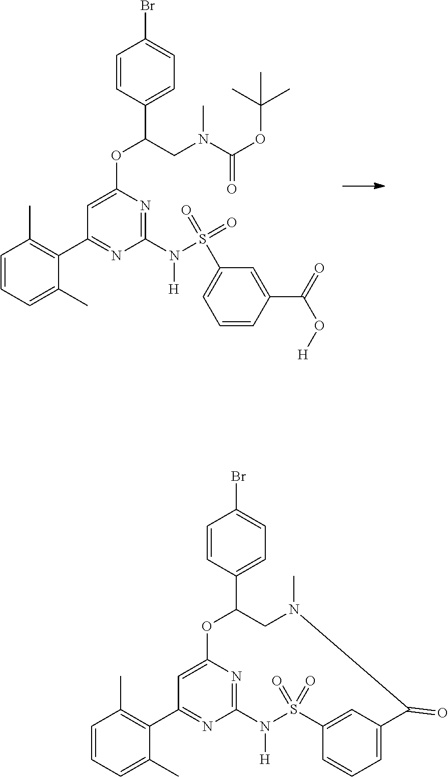 [SMILES: Cc1cccc(C)c1-c1cc(OC(CN(C)C(=O)OC(C)(C)C)c2ccc(Br)cc2)nc(NS(=O)(=O)c2cccc(C(=O)O)c2)n1 ; Cc1cccc(C)c1-c1cc2nc(n1)NS(=O)(=O)c1cccc(c1)C(=O)N(C)CC(c1ccc(Br)cc1)O2]

To a nitrogen sparged round bottom flask was added 3-[[4-[1-(4-bromophenyl)-2-[tert-butoxycarbonyl(methyl)amino]ethoxy]-6-(2,6-dimethylphenyl)pyrimidin-2-yl]sulfamoyl]benzoic acid (634 mg, 0.8909 mmol) and a solution of HCl (2.2 mL of 4 M, 8.800 mmol) in dioxane. The solution was allowed to stir at 40° C. for 40 min. The reaction solution was concentrated in vacuo to afford an off white residue taken directly into the next step. To the material was added HATU (410 mg, 1.078 mmol), anhydrous DMF (30 mL), and DIEA (0.5 mL, 2.871 mmol). The reaction solution was stirred at room temperature for 2 h. The reaction mixture was partitioned between ethyl acetate and a 1 M HCl solution. The organics were separated, washed with brine, dried over sodium sulfate and evaporated. The crude material was chromatographed thru a silica gel column using a gradient of 100% DCM to 15% MeOH/DCM to afford 10-(4-bromophenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (340 mg, 61%)¹H NMR (400 MHz, DMSO-d₆) δ 8.67 (s, 1H), 7.95 (s, 1H), 7.78-7.57 (m, 6H), 7.24 (d, J 7.9 Hz, 1H), 7.11 (d, J 7.6 Hz, 2H), 6.38 (d, J 10.7 Hz, 2H), 3.75 (d, J 14.6 Hz, 1H), 3.43 (dd, J 14.3, 10.9 Hz, 1H), 2.29 (s, 3H), 2.01 (d, J 17.9 Hz, 6H). ESI-MS m/z calc. 592.078, found 592.9 (M+1)+, Retention time: 0.66 minutes (LC method D).

### Step 4: 6-(2,6-Dimethylphenyl)-12-methyl-2,2-dioxo-10-[4-(4-pyridyl)phenyl]-9-oxa-2λ⁶-thia-3,5,12,19-tetrazatricyclo[12.3.1.14,8]nonadeca-1(18),4(19),5,7,14,16-hexaen-13-one (Compound 9)

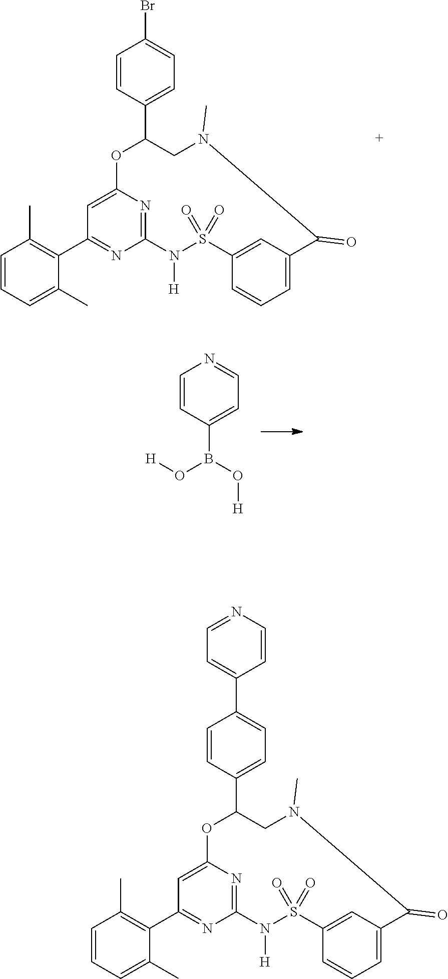 [SMILES: Cc1cccc(C)c1-c1cc2nc(n1)NS(=O)(=O)c1cccc(c1)C(=O)N(C)CC(c1ccc(Br)cc1)O2 ; OB(O)c1ccncc1 ; Cc1cccc(C)c1-c1cc2nc(n1)NS(=O)(=O)c1cccc(c1)C(=O)N(C)CC(c1ccc(-c3ccncc3)cc1)O2]

To a nitrogen sparged round bottom flask was added 10-(4-bromophenyl)-6-(2,6-dimethylphenyl)-12-methyl-2,2-dioxo-9-oxa-2?⁶-thia-3,5,12,19-tetrazatricyclo[12.3.1.14,8]nonadeca-1(18),4(19),5,7,14,16-hexaen-13-one (52 mg, 0.08762 mmol), 4-pyridylboronic acid (21.54 mg, 0.1752 mmol), potassium carbonate (36.33 mg, 0.2629 mmol), Pd(dppf)Cl₂ (14.31 mg, 0.01752 mmol), dioxane (1.5 mL), and water (0.5 mL). The reaction mixture was stirred for 2 h under nitrogen at 80° C. The reaction solution was filtered through a Celite padded funnel and the mother liquor was evaporated in vacuo to dryness. The residue was purified by reverse phase HPLC using a gradient off 1% MeCN in water to 99% MeCN over 15 min to afford 6-(2,6-dimethylphenyl)-12-methyl-2,2-dioxo-10-[4-(4-pyridyl)phenyl]-9-oxa-2?⁶-thia-3,5,12,19-tetrazatricyclo[12.3.1.14,8]nonadeca-1(18),4(19),5,7,14,16-hexaen-13-one (28 mg, 53%)¹H NMR (400 MHz, DMSO-d₆) δ 9.01-8.87 (m, 2H), 8.74 (s, 1H), 8.37 (d, J 6.0 Hz, 2H), 8.18 (d, J 8.3 Hz, 2H), 8.00 (d, J 8.3 Hz, 2H), 7.95 (d, J 7.2 Hz, 1H), 7.69 (d, J 7.9 Hz, 2H), 7.31-7.21 (m, 1H), 7.12 (d, J 7.7 Hz, 2H), 6.49 (dd, J=10.6, 4.4 Hz, 1H), 6.37 (s, 1H), 3.82 (dd, J=14.2, 4.2 Hz, 1H), 2.31 (s, 3H), 2.05 (s, 6H). ESI-MS m/z calc. 591.19403, found 592.35 (M+1)⁺; Retention time: 1.11 minutes; LC method A.

### Example 10: Preparation of Compound 10, Compound 11, Compound 12, and Compound

13

### Step 1: 3-[[4-[2-[tert-Butoxycarbonyl(methyl)amino]-2-(4-tert-butylphenyl)ethoxy]-6-(2,6-dimethylphenyl)pyrimidin-2-yl]sulfamoyl]benzoic acid

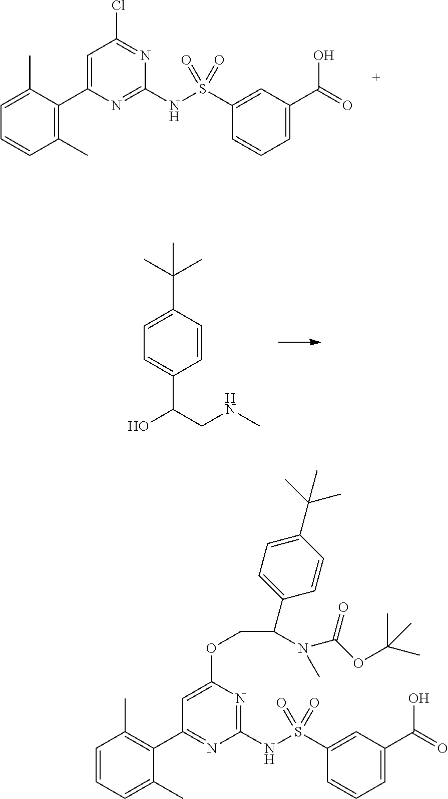 [SMILES: Cc1cccc(C)c1-c1cc(Cl)nc(NS(=O)(=O)c2cccc(C(=O)O)c2)n1 ; CNCC(O)c1ccc(C(C)(C)C)cc1 ; Cc1cccc(C)c1-c1cc(OCC(c2ccc(C(C)(C)C)cc2)N(C)C(=O)OC(C)(C)C)nc(NS(=O)(=O)c2cccc(C(=O)O)c2)n1]

In a 250 mL flask, 3-[[4-chloro-6-(2,6-dimethylphenyl)pyrimidin-2-yl]sulfamoyl]benzoic acid (1.7 g, 4.068 mmol), 1-(4-tert-butylphenyl)-2-(methylamino)ethanol (845 mg, 4.076 mmol) and THE (35.0 mL) were mixed and cooled in an ice bath at 0° C., to which KOtBu (2.75 g, 24.51 mmol) was added. This mixture was stirred 30 min at 0° C., and di-tert-butyl dicarbonate (approximately 2.396 g, 2.522 mL, 10.98 mmol) was added allowed to stir for 16 hours. The mixture was then diluted with ethyl acetate and quenched with sat ammonium chloride solution and then extracted with additional ethyl acetate (2×75 mL). The combined organic extracts were washed with water (50 mL) and saturated brine solution (50 mL), then dried over sodium sulfate, filtered, and evaporated in vacuo. This crude product was purified on silica gel chromatography (120 gram column) using a gradient from 100% dichloromethane to 20% methanol in dichloromethane followed by a second silica gel chromatography (80 gram column) using a gradient from 100% dichloromethane to 10% methanol in dichloromethane to afford as a white solid 3-[[4-[2-[tert-butoxycarbonyl(methyl)amino]-1-(4-tert-butylphenyl)ethoxy]-6-(2,6-dimethylphenyl)pyrimidin-2-yl]sulfamoyl]benzoic acid (0.65 g, 23%) ESI-MS m/z calc. 688.2931, found 689.6 (M+1)⁺; Retention time: 2.18 minutes; LC method A.

### Step 2: 10-(4-tert-butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 10), and 11-(4-tert-butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 13)

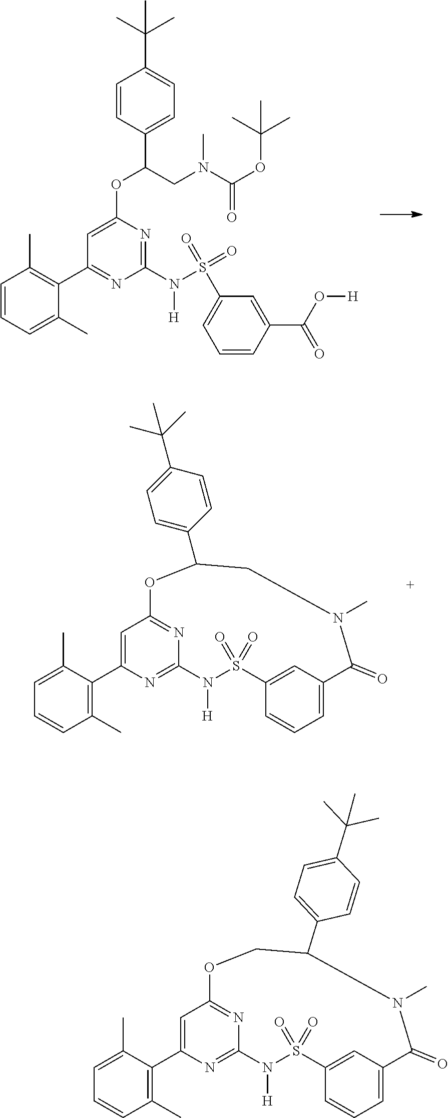 [SMILES: Cc1cccc(C)c1-c1cc(OC(CN(C)C(=O)OC(C)(C)C)c2ccc(C(C)(C)C)cc2)nc(NS(=O)(=O)c2cccc(C(=O)O)c2)n1 ; Cc1cccc(C)c1-c1cc2nc(n1)NS(=O)(=O)c1cccc(c1)C(=O)N(C)CC(c1ccc(C(C)(C)C)cc1)O2 ; Cc1cccc(C)c1-c1cc2nc(n1)NS(=O)(=O)c1cccc(c1)C(=O)N(C)C(c1ccc(C(C)(C)C)cc1)CO2]

In a 250-mL flask, 3-[[4-[2-[tert-butoxycarbonyl(methyl)amino]-1-(4-tert-butylphenyl)ethoxy]-6-(2,6-dimethylphenyl)pyrimidin-2-yl]sulfamoyl]benzoic acid (550 mg, 0.7985 mmol) was dissolved in DCM (30 mL) and to the mixture was added HCl (4M in dioxane) (2.5 mL of 4 M, 10.00 mmol) and it was stirred at room temperature for 90 min. The reaction mixture was concentrated under reduced pressure to a white solid, which was then purified by reverse-phase preparative chromatography utilizing a C₁₈ column a 1-50% gradient of 15 min of acetonitrile-in water+5 mmol HCl to give a mixture of 3-[[4-[1-(4-tert-butylphenyl)-2-(methylamino)ethoxy]-6-(2,6-dimethylphenyl)pyrimidin-2-yl]sulfamoyl]benzoic acid (HCl salt) ESI-MS m/z calc. 588.24066, found 589.3 (M+1)⁺; Retention time: 1.29 minutes and 3-[[4-[2-(4-tert-butylphenyl)-2-(methylamino)ethoxy]-6-(2,6-dimethylphenyl)pyrimidin-2-yl]sulfamoyl]benzoic acid (HCl salt) ESI-MS m/z calc. 588.24066, found 589.3 (M+1)⁺ Retention time: 1.36 minutes (LC method A).

The solid mixture (240 mg, HCl salt) was dissolved in DMF (25 mL), to which DIPEA (1.0 mL, 5.741 mmol) and HATU (450 mg, 1.183 mmol) were added. After stirring at room temperature for 15 min, the reaction mixture was diluted with water and filtered. This crude product was purified by reverse-phase preparative chromatography utilizing a C₁₈ column (30-99% over 15 min of acetonitrile in water+5 mM HCl) to give, as an off-white solid 10-(4-tert-butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (85.7 mg, 19%) ESI-MS m/z calc. 570.2301, found 571.3 (M+1)⁺; Retention time: 1.95 minutes (LC method A), ¹H NMR (400 MHz, DMSO-d₆) δ 13.00 (s, 1H), 8.64 (s, 1H), 7.93 (d, J 7.0 Hz, 1H), 7.71-7.58 (m, 4H), 7.54 (d, J 8.4 Hz, 2H), 7.25 (t, J 7.6 Hz, 1H), 7.11 (d, J 7.6 Hz, 2H), 6.40-6.24 (m, 2H), 3.76 (dd, J 14.3, 4.1 Hz, 1H), 3.43 (dd, J 14.2, 10.8 Hz, 1H), 2.25 (s, 3H), 2.04 (s, 6H), 1.32 (s, 9H); and as an off-white solid, 11-(4-tert-butylphenyl)-6-(2,6-dimethylphenyl)-12-methyl-2,2-dioxo-9-oxa-2?⁶-thia-3,5,12,19-tetrazatricyclo[12.3.1.14,8]nonadeca-1(18),4(19),5,7,14,16-hexaen-13-one (85.0 mg, 19%), ESI-MS m/z calc. 570.2301, found 571.3 (M+1)⁺; Retention time: 1.87 minutes, (LC method A), ¹H NMR (400 MHz, DMSO-d₆) δ 12.98 (s, 1H), 8.60 (s, 1H), 8.06-7.66 (m, 3H), 7.44-7.31 (m, 2H), 7.19 (dd, J=11.0, 8.0 Hz, 3H), 7.07 (s, 2H), 6.18 (s, 1H), 5.40 (dd, J 11.7, 4.4 Hz, 1H), 5.18 (t, J 11.6 Hz, 1H), 4.95 (dd, J 11.7, 4.5 Hz, 1H), 2.60 (s, 3H), 1.97 (d, J 65.7 Hz, 6H), 1.24 (d, J 1.2 Hz, 9H).

### Step 3: 10-(4-tert-Butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one, enantiomer 1 (Compound 11), and 10-(4-tert-butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one, enatiomer 2 (Compound 12)

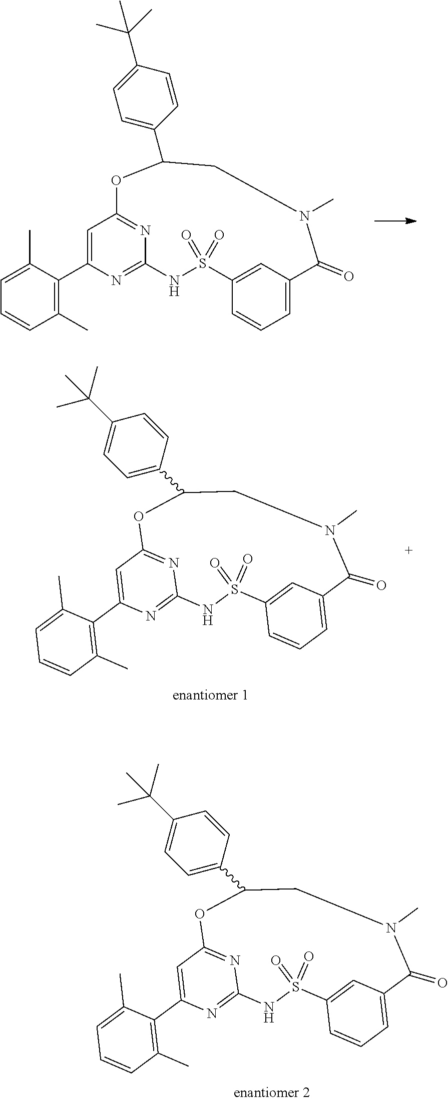

Racemic 10-(4-tert-Butylphenyl)-6-(2,6-dimethylphenyl)-12-methyl-2,2-dioxo-9-oxa-2λ⁶-thia-3, 5,12,19-tetrazatricyclo[12.3.1.14,8]nonadeca-1(18),4(19),5,7,14,16-hexaen-13-one was separated by chiral SFC using IA column to give two enantiomers: Enantiomer 1, SFC Peak 1 10-(4-tert-butylphenyl)-6-(2,6-dimethylphenyl)-12-methyl-2,2-dioxo-9-oxa-2?⁶-thia-3,5,12,19-tetrazatricyclo[12.3.1.14,8]nonadeca-1(18),4(19),5,7,14,16-hexaen-13-one (35.3 mg, 87%) as a white solid. ESI-MS m/z calc. 570.2301, found 571.3 (M+1)⁺; Retention time: 1.95 minutes. (LC method A), ¹H NMR (400 MHz, DMSO-d₆) δ 13.02 (s, 1H), 8.63 (s, 1H), 7.93 (d, J 6.8 Hz, 1H), 7.71-7.60 (m, 4H), 7.57-7.51 (m, 2H), 7.25 (t, J 7.6 Hz, 1H), 7.11 (d, J 7.6 Hz, 2H), 6.43-6.18 (m, 2H), 3.84-3.70 (m, 1H), 3.43 (dd, J 14.3, 10.8 Hz, 1H), 2.25 (s, 3H), 2.06 (d, J 15.6 Hz, 6H), 1.32 (s, 9H); and enantiomer 2, SFC Peak 2, 10-(4-tert-butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (34.8 mg, 87%) as a white solid, ESI-MS m/z calc. 570.2301, found 571.3 (M+1)⁺; Retention time: 1.95 minutes, (LC method A). ¹H NMR (400 MHz, DMSO-d₆) δ 13.02 (s, 1H), 8.64 (s, 1H), 7.93 (d, J 7.0 Hz, 1H), 7.72-7.59 (m, 4H), 7.58-7.51 (m, 2H), 7.25 (t, J 7.6 Hz, 1H), 7.11 (d, J 7.7 Hz, 2H), 6.45-6.19 (m, 2H), 3.82-3.70 (m, 1H), 3.44 (dd, J 14.3, 10.9 Hz, 1H), 2.25 (s, 3H), 2.04 (s, 6H), 1.32 (s, 9H).

### Example 11: Preparation of Compound 14

### Step 1: 3-[[4-[2-(tert-Butoxycarbonylamino)-1-(4-tert-butylphenyl)ethoxy]-6-(2,6-dimethylphenyl)pyrimidin-2-yl]sulfamoyl]benzoic acid

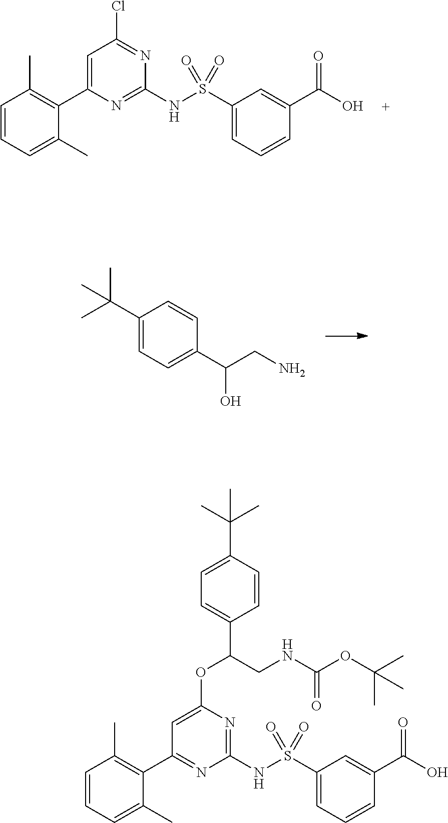 [SMILES: Cc1cccc(C)c1-c1cc(Cl)nc(NS(=O)(=O)c2cccc(C(=O)O)c2)n1 ; CC(C)(C)c1ccc(C(O)CN)cc1 ; Cc1cccc(C)c1-c1cc(OC(CNC(=O)OC(C)(C)C)c2ccc(C(C)(C)C)cc2)nc(NS(=O)(=O)c2cccc(C(=O)O)c2)n1]

To a solution of 3-[[4-chloro-6-(2,6-dimethylphenyl)pyrimidin-2-yl]sulfamoyl]benzoic acid (1.22 g, 2.920 mmol) and 2-amino−1-(4-tert-butylphenyl)ethanol (0.66 g, 3.415 mmol) in THF (24 mL) at 0° C. was added sodium tert-butoxide (0.85 g, 8.845 mmol) and the reaction was stirred at this temperature for 1 hour. The cooling bath was removed, and the reaction was stirred at room temperature for an additional 2 hours. At this time, tert-butoxycarbonyl tert-butyl carbonate (790 mg, 3.620 mmol) was added and the reaction mixture was stirred at room temperature for 16 hours. The reaction mixture was poured into water, the pH adjusted to ˜5 with 1N HCl and extracted with EtOAc (3×). The organics were combined, washed brine, dried over sodium sulfate and evaporated to dryness. Purification by silica gel column chromatography (40 g silica 0-50% EtOAc in hexanes) gave 3-[[4-[2-(tert-butoxycarbonylamino)-1-(4-tert-butylphenyl)ethoxy]-6-(2,6-dimethylphenyl)pyrimidin-2-yl]sulfamoyl]benzoic acid (316 mg, 16%) as a white solid. ESI-MS m/z calc. 674.2774, found 675.5 (M+1)⁺; Retention time: 0.78 minutes, LC method D.

### Step 2: 10-(4-tert-butylphenyl)-12-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 14)

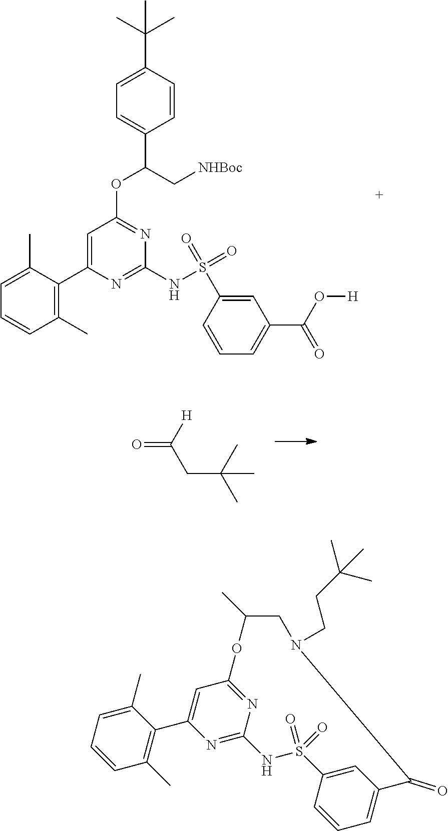

To a solution of 3-[[4-[2-(tert-butoxycarbonylamino)-1-(4-tert-butylphenyl)ethoxy]-6-(2,6-dimethylphenyl)pyrimidin-2-yl]sulfamoyl]benzoic acid (316 mg, 0.4683 mmol) in DCM (5 mL) was added HCl (4N in Dioxane) (5 mL of 4 M, 20.00 mmol) and the reaction mixture stirred at room temperature for 1 h. The reaction mixture was evaporated then co-evaporated with DCM to give 3-[[4-[2-amino−1-(4-tert-butylphenyl)ethoxy]-6-(2,6-dimethylphenyl)pyrimidin-2-yl]sulfamoyl]benzoic acid (Hydrochloride salt) (271 mg, 95%) as a white solid. ESI-MS m/z calc. 574.225, found 575.4 (M+1)⁺; Retention time: 0.54 minutes (LC method D). To a solution of 3-[[4-[2-amino-1-(4-tert-butylphenyl)ethoxy]-6-(2,6-dimethylphenyl)pyrimidin-2-yl]sulfamoyl]benzoic acid (50 mg, 0.08700 mmol) and 3,3-dimethylbutanal (8.7 mg, 0.08686 mmol) in DCM (25 μL) was added sodium triacetoxyborohydride (92 mg, 0.4341 mmol). The solution was stirred for 1 h. The volatiles were removed under a steady stream of air. The sample was purified by reverse phase HPLC (Phenomenex Luna C₁₈ column (75×30 mm, 5 μm particle size), gradient: 1-99% acetonitrile in water (5 mM HCl) over 15.0 minutes) which afforded the intermediate 3-[[4-[1-(4-tert-butylphenyl)-2-(3,3-dimethylbutylamino)ethoxy]-6-(2,6-dimethylphenyl)pyrimidin-2-yl]sulfamoyl]benzoic acid. To that intermediate in DMF (60 μL) was added HATU (33 mg, 0.08679 mmol). The reaction was stirred for 5 min before adding triethylamine (12 μL, 0.08610 mmol). The reaction was further stirred for 20 min. The sample was purified by reverse phase HPLC (Phenomenex Luna C₁₈ column (75×30 mm, 5 μm particle size), gradient: 1-99% acetonitrile in water (5 mM HCl) over 15.0 minutes) which afforded 10-(4-tert-butylphenyl)-12-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (3.4 mg, 6%) ESI-MS m/z calc. 640.30835, found 641.2 (M+1)⁺; Retention time: 2.39 minutes; LC method A.

### Example 12: Preparation of Compound 15, Compound 16, and Compound 17

### Step 1: tert-Butyl N-[2-(3-tert-butylphenyl)-2-hydroxy-ethyl]-N-methyl-carbamate

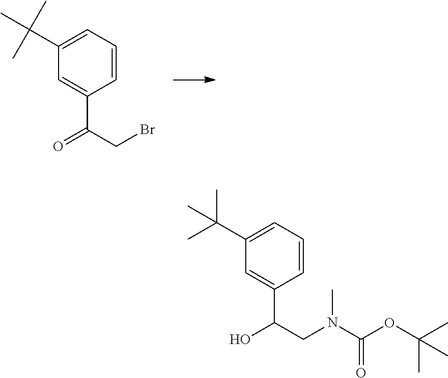 [SMILES: CC(C)(C)c1cccc(C(=O)CBr)c1 ; CN(CC(O)c1cccc(C(C)(C)C)c1)C(=O)OC(C)(C)C]

To a EtOH solution of methanamine (3.7 g of 33% w/w, 39.31 mmol) was added a solution of 2-bromo-1-(3-tert-butylphenyl)ethanone (500 mg, 1.960 mmol) in EtOH (5 mL) at RT slowly with vigorous stirring. The mixture was stirred at RT for 1 h. Next, sodium borohydride (223 mg, 5.894 mmol) was added portion wise and the mixture was stirred overnight. The mixture was evaporated in vacuo to afford crude 1-(3-tert-butylphenyl)-2-(methylamino)ethanol (400 mg, 59%) ESI-MS m/z calc. 207.16231, found 208.19 (M+1)⁺; Retention time: 0.38 minutes (LC method D). The crude material was dissolved in THE (10 mL) and treated with Boc anhydride (500 mg, 2.291 mmol) at RT. After 3 h the mixture was evaporated, quenched with aqueous sodium bicarbonate and extracted with EtOAc. The organic phase was dried over sodium sulfate, evaporated and purified by silica gel chromatography using 12 g column (eluent hexanes:EtOAc 100:0% to 70:30%) to afford tert-butyl N-[2-(3-tert-butylphenyl)-2-hydroxy-ethyl]-N-methyl-carbamate (240 mg, 40%) ESI-MS m/z calc. 307.21475, found 308.29 (M+1)⁺; Retention time: 0.73 minutes, LC method D.

### Step 2: 3-[[4-[2-[tert-Butoxycarbonyl(methyl)amino]-1-(3-tert-butylphenyl)ethoxy]-6-(2,6-dimethylphenyl)pyrimidin-2-yl]sulfamoyl]benzoic acid

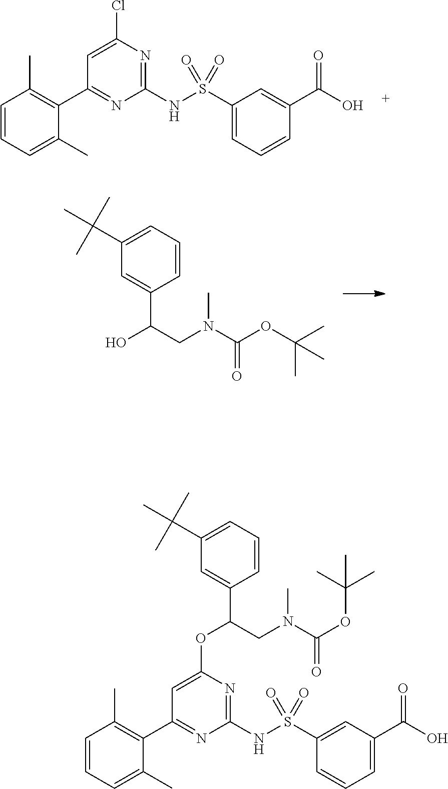 [SMILES: Cc1cccc(C)c1-c1cc(Cl)nc(NS(=O)(=O)c2cccc(C(=O)O)c2)n1 ; CN(CC(O)c1cccc(C(C)(C)C)c1)C(=O)OC(C)(C)C ; Cc1cccc(C)c1-c1cc(OC(CN(C)C(=O)OC(C)(C)C)c2cccc(C(C)(C)C)c2)nc(NS(=O)(=O)c2cccc(C(=O)O)c2)n1]

tert-Butyl N-[2-(3-tert-butylphenyl)-2-hydroxy-ethyl]-N-methyl-carbamate (240 mg, 0.7807 mmol) was dissolved in dioxane solution of HCl (3.6 mL of 4 M, 14.40 mmol) and stirred at RT for 3h. The mixture was evaporated and dried in vacuo to give a crude amino alcohol. A solution of this amino alcohol and 3-[[4-chloro-6-(2,6-dimethylphenyl)pyrimidin-2-yl]sulfamoyl]benzoic acid (294 mg, 0.7036 mmol) in THE (7 mL) was cooled in the ice bath and treated with sodium tert-butoxide (380 mg, 3.954 mmol). The mixture was stirred at RT for 3 h. The mixture was treated with Boc anhydride (155 mg, 0.7102 mmol) and stirred at RT for 3 h. The mixture was quenched with aqueous ammonium chloride and extracted with EtOAc. The organic phase was dried over sodium sulfate and evaporated. The residue was purified by preparative reverse phase HPLC (C₁₈): 1-99% ACN in water/HCl modifier (15 min) to afford 3-[[4-[2-[tert-butoxycarbonyl(methyl)amino]-1-(3-tert-butylphenyl)ethoxy]-6-(2,6-dimethylphenyl)pyrimidin-2-yl]sulfamoyl]benzoic acid (101.7 mg, 21%) ESI-MS m/z calc. 688.2931, found 689.2 (M+1)⁺; Retention time: 0.8 minutes; LC method D.

### Step 3: 10-(3-tert-butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one, racemic mixture (Compound 16), and 10-(3-tert-butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one, enantiomer 1 (Compound 15), and 10-(3-tert-butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one, enantiomer 2 (Compound 17)

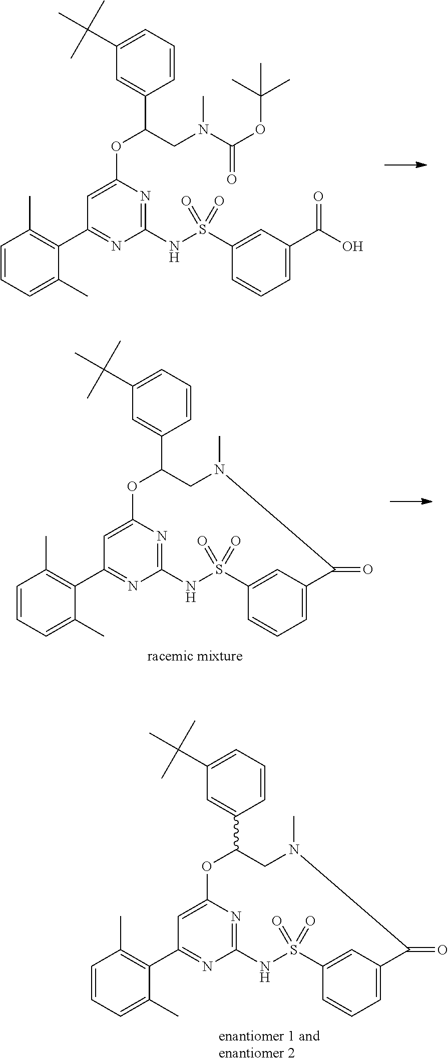 [SMILES: Cc1cccc(C)c1-c1cc(OC(CN(C)C(=O)OC(C)(C)C)c2cccc(C(C)(C)C)c2)nc(NS(=O)(=O)c2cccc(C(=O)O)c2)n1 ; Cc1cccc(C)c1-c1cc2nc(n1)NS(=O)(=O)c1cccc(c1)C(=O)N(C)CC(c1cccc(C(C)(C)C)c1)O2 ; Cc1cccc(C)c1-c1cc2nc(n1)NS(=O)(=O)c1cccc(c1)C(=O)N(C)CC(c1cccc(C(C)(C)C)c1)O2]

3-[[4-[2-[tert-butoxycarbonyl(methyl)amino]-1-(3-tert-butylphenyl)ethoxy]-6-(2,6-dimethylphenyl)pyrimidin-2-yl]sulfamoyl]benzoic acid (101 mg, 0.1466 mmol) was treated with a dioxane solution of HCl (1000 μL of 4 M, 4.000 mmol), stirred at RT for 3 h, evaporated and dried in vacuo. The intermediate was dissolved in DMF (4.5 mL) and treated with HATU (84 mg, 0.2209 mmol) and DIPEA (80 μL, 0.4593 mmol) at 0° C. The mixture was stirred for 1 h at RT, filtered and purified by preparative reverse phase HPLC (C₁₈): 1-99% ACN in water/HCl modifier (15 min) to afford racemic 10-(3-tert-butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (43.4 mg, 51%) racemic mixture ESI-MS m/z calc. 570.2301, found 571.38 (M+1)⁺; Retention time: 2.0 minutes (LC method A).

Two enantiomers were separated by chiral SFC ((R,R)-Whelk-O (150×2.1 mm), 3 m column, mobile phase: 80% MeOH with NH₃, 40 mL/min, injection volume 400 μL, 220/224 nm) to give: enantiomer 1, peak 1:10-(3-tert-butylphenyl)-6-(2,6-dimethylphenyl)-12-methyl-2,2-dioxo-9-oxa-2?⁶-thia-3,5,12,19-tetrazatricyclo[12.3.1.14,8]nonadeca-1(18),4(19),5,7,14,16-hexaen-13-one (16.1 mg, 19%); ESI-MS m/z calc. 570.2301, found 571.38 (M+1)⁺; Retention time: 2.0 minutes, LC method A; and enantiomer 2, peak 2, 10-(3-tert-butylphenyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (16.3 mg, 19%); ESI-MS m/z calc. 570.2301, found 571.38 (M+1)⁺; Retention time: 2.0 minutes. LC method A.

### Example 13: Preparation of Compound 18

### Step 1: tert-Butyl N-benzyl-N-[(2R)-2-hydroxy-2-phenylethyl]carbamate

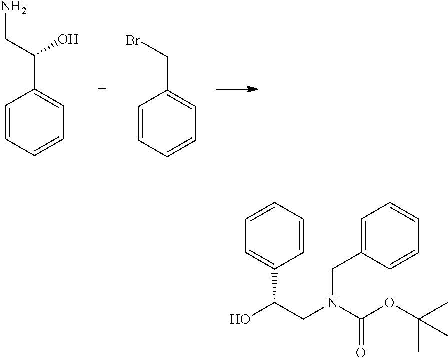 [SMILES: NC[C@H](O)c1ccccc1 ; BrCc1ccccc1 ; CC(C)(C)OC(=O)N(Cc1ccccc1)C[C@H](O)c1ccccc1]

(1R)-2-Amino-1-phenyl-ethanol (274.4 mg, 2 mmol) was dissolved in tetrahydrofuran. bromomethylbenzene (approximately 393.4 mg, 273.6 μL, 2.300 mmol) was added followed by solid potassium carbonate (approximately 276.4 mg, 2.000 mmol). The reaction mixture was allowed to stir at room temperature for 2 hours. Di-tert-butyl dicarbonate (approximately 436.5 mg, 459.5 μL, 2.000 mmol) was then added. The reaction mixture was allowed to stir overnight at room temperature. The reaction mixture was diluted with DCM (7 mL) and washed with aqueous HCl (1 M, 1×7 mL) and brine (2×7 mL). The final organic layer was dried over sodium sulfate. The crude product was chromatographed on a 12-gram silica gel column eluting with a 0-40% EtOAc/hexane gradient over 30 minutes. tert-Butyl N-benzyl-N-[(2R)-2-hydroxy-2-phenylethyl]carbamate (87 mg, 13%) was obtained. ESI-MS m/z calc. 327.18344, found 328.0 (M+1)⁺; Retention time: 1.89 minutes; LC method A.

### Step 2: 3-[[4-[(1R)-2-[Benzyl(tert-butoxycarbonyl)amino]-1-phenyl-ethoxy]-6-(2,6-dimethylphenyl)pyrimidin-2-yl]sulfamoyl]benzoic acid

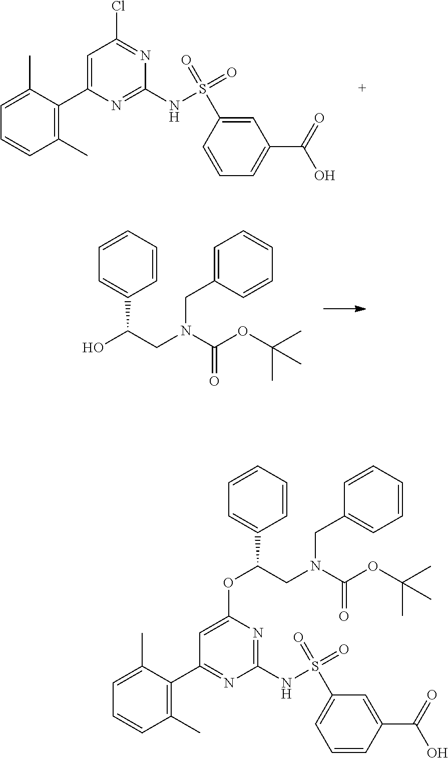 [SMILES: Cc1cccc(C)c1-c1cc(Cl)nc(NS(=O)(=O)c2cccc(C(=O)O)c2)n1 ; CC(C)(C)OC(=O)N(Cc1ccccc1)C[C@H](O)c1ccccc1 ; Cc1cccc(C)c1-c1cc(O[C@@H](CN(Cc2ccccc2)C(=O)OC(C)(C)C)c2ccccc2)nc(NS(=O)(=O)c2cccc(C(=O)O)c2)n1]

3-[[4-Chloro-6-(2,6-dimethylphenyl)pyrimidin-2-yl]sulfamoyl]benzoic acid (approximately 34.04 mg, 0.08147 mmol) was dissolved into a solution of tert-butyl N-benzyl-N-[(2R)-2-hydroxy-2-phenylethyl]carbamate (approximately 40.01 mg, 0.1222 mmol) in tetrahydrofuran (2 mL). Solid sodium tert-butoxide (approximately 39.14 mg, 0.4073 mmol) was added. The reaction mixture was allowed to stir at room temperature overnight. The reaction mixture was diluted with DCM (7 mL) and washed with aqueous HCl (1 M, 1×7 mL) and brine (2×7 mL). The organic layer was dried over sodium sulfate, filtered and concentrated under reduced pressure. The product, 3-[[4-[(1R)-2-[benzyl(tert-butoxycarbonyl)amino]-1-phenyl-ethoxy]-6-(2,6-dimethylphenyl)pyrimidin-2-yl]sulfamoyl]benzoic acid was used in the next step without further purification (191 mg, impure material). ESI-MS m/z calc. 708.2618, found 709.0 (M+1)⁺; Retention time: 2.187 minutes; LC method A.

### Step 3: (10R)-12-Benzyl-6-(2,6-dimethylphenyl)-2,2-dioxo-10-phenyl-9-oxa-2λ⁶-thia-3,5,12,19-tetrazatricyclo[12.3.1.14,8]nonadeca-1(18),4(19),5,7,14,16-hexaen-13-one (Compound 18)

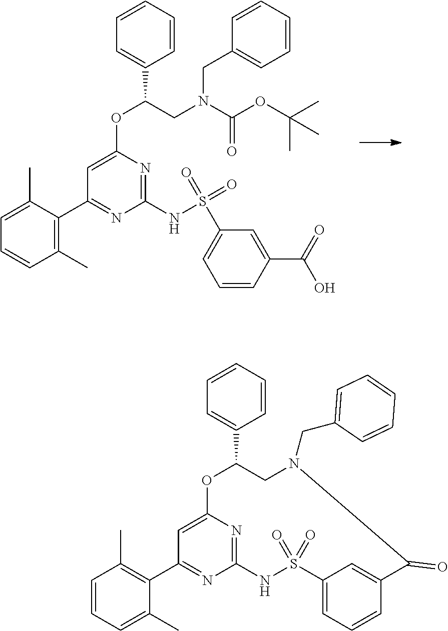 [SMILES: Cc1cccc(C)c1-c1cc(O[C@@H](CN(Cc2ccccc2)C(=O)OC(C)(C)C)c2ccccc2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccccc1)C[C@@H](c1ccccc1)O2]

3-[[4-[(1R)-2-[benzyl(tert-butoxycarbonyl)amino]-1-phenyl-ethoxy]-6-(2,6-dimethylphenyl)pyrimidin-2-yl]sulfamoyl]benzoic acid was dissolved in a solution of HCl in dioxane. The solution was allowed to stir at room temperature for 15 minutes. Volatiles were removed under reduced pressure. The remaining oil was dissolved in DMF (0.7 mL), and HATU was added followed by triethylamine. The reaction mixture was allowed to stir for an additional 15 minutes. After filtration, the product was isolated by reverse-phase HPLC using a Luna Cis (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. to provide (10R)-12-benzyl-6-(2,6-dimethylphenyl)-2,2-dioxo-10-phenyl-9-oxa-2λ⁶-thia-3,5,12,19-tetrazatricyclo[12.3.1.14,8]nonadeca-1(18),4(19),5,7,14,16-hexaen-13-one (6.1 mg), ESI-MS m/z calc. 590.1988, found 591.3 (M+1)⁺; Retention time: 1.99 minutes; LC method A.

### Example 14: Preparation of Compound 19 and Compound 20

### Step 1: 2-Methylhept-6-en-2-ol

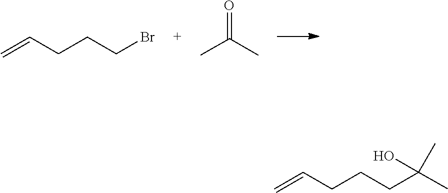

5-Bromo-pent-1-ene (18.870 g, 15 mL, 126.62 mmol) in diethyl ether (32 mL) was dropwise added to magnesium turnings (3.2 g, 131.66 mmol) in diethyl ether (190 mL). The reaction mixture was stirred at 45° C. for 4 h. After the solution reached room temperature, acetone (11.470 g, 14.5 mL, 197.49 mmol) in diethyl ether (32 mL) was added dropwise at 0° C. to the reaction mixture, which was allowed to stir overnight at ambient temperature. The mixture was poured into ice, and diluted HCl (60 mL) and aqueous NaHSO₄ (1.0 M, 50 mL) were added. The aqueous layer was extracted with diethyl ether (3×100 mL). The combined organic layers were dried over sodium sulfate, filtered and the solvent was removed by rotary evaporation to give 2-methylhept-6-en-2-ol (23 g, 95%), as a colorless oil. ¹H NMR (400 MHz, CDCl₃) δ 5.88-5.76 (m, 1H), 5.06-4.93 (m, 2H), 2.11-2.01 (m, 2H), 1.49-1.44 (m, 4H), 1.22-1.21 (m, 6H).

### Step 2: (6,6-Dimethyltetrahydropyran-2-yl)methanol

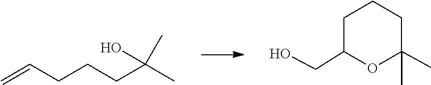

3-Chloroperoxybenzoic acid (22 g, 94.341 mmol) was added in portions to 2-methylhept-6-en-2-ol (10 g, 77.996 mmol) in dichloromethane (190 mL). The mixture was stirred at room temperature for 18 h. The precipitated acid was removed by filtration and the solution was concentrated under reduced pressure. Pentane (20 mL) was added to the residue then the precipitated acid was removed by filtration. The solution was concentrated under reduced pressure. Dichloromethane (670 mL) then methanesulfonic acid (3.7025 g, 2.5 mL, 38.525 mmol) were added. The mixture was stirred at room temperature for 1 h. Saturated aqueous sodium bicarbonate (50 mL) was added, the phases were separated, and the product was extracted with DCM (2×60 mL). The combined organic layers were washed with aqueous sodium bicarbonate (2×40 mL), brine (50 mL) then dried over sodium sulfate, filtered and concentrated under reduced pressure. Afforded (6,6-dimethyltetrahydropyran-2-yl)methanol (7.3 g, 45%) as a light yellow oil. ¹H NMR (400 MHz, CDCl₃) δ 3.72-3.65 (m, 1H), 3.61-3.53 (m, 1H), 3.49-3.41 (m, 1H), 1.74-1.64 (m, 2H), 1.53-1.44 (m, 2H), 1.43-1.26 (m, 2H), 1.23-1.22 (m, 3H), 1.22-1.21 (m, 3H).

### Step 3: 6,6-Dimethyltetrahydropyran-2-carbaldehyde

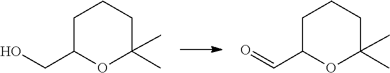

A solution of (6,6-dimethyltetrahydropyran-2-yl)methanol (300 mg, 2.0803 mmol) in dichloromethane (3 mL) was cooled to −10° C. A solution of sodium bromide (22 mg, 0.2138 mmol) and sodium bicarbonate (105 mg, 1.2499 mmol) dissolved in water (0.65 mL) was added. After 15 minutes of stirring at ˜10° C., 2,2,6,6-tetramethylpiperidine 1-oxyl (3.5 mg, 0.0224 mmol) was added, followed by the slow addition of sodium hypochlorite (1.505 M in water) (1.5 mL of 1.505 M, 2.2575 mmol) dropwise keeping the internal temperature in the −10−8° C. range. The mixture was stirred until the layers separated. Methanol (395.50 mg, 0.5 mL, 12.343 mmol) was added to the mixture. The organic layer was separated, and the aqueous layer was extracted with dichloromethane (2×20 mL). The combined organic layers were washed with saturated aqueous NaCl (20 mL), dried over sodium sulfate, filtered, and concentrated. The crude product was purified on silica-gel (24 g) eluting with a gradient of diethyl ether in pentane 10% for 3 CV then 20% 3 CV then 40% for 6 CV to yield 6,6-dimethyltetrahydropyran-2-carbaldehyde (90 mg, 23%) as a brown oil. ¹H NMR (400 MHz, CDCl₃) δ 9.60 (s, 1H), 4.05-3.98 (m, 1H), 1.84-1.65 (m, 3H), 1.56-1.37 (m, 3H), 1.30 (s, 3H), 1.24 (s, 3H). GC-FID (GC method 1B): Retention time: 3.41 minutes.

### Step 4: 3-[[4-(2,6-Dimethylphenyl)-6-[(2R)-2-[(6,6-dimethyltetrahydropyran-2-yl)methylamino]-4,4-dimethyl-pentoxy]pyrimidin-2-yl]sulfamoyl]benzoic acid

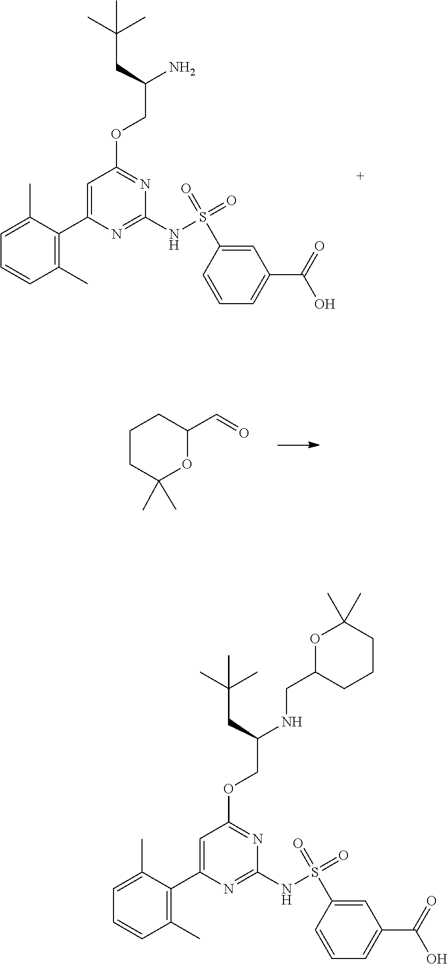

3-[[4-[(2R)-2-amino-4,4-dimethyl-pentoxy]-6-(2,6-dimethylphenyl)pyrimidin-2-yl]sulfamoyl]benzoic acid (hydrochloride salt) (100 mg, 0.1821 mmol) and 6,6-dimethyltetrahydropyran-2-carbaldehyde (31 mg, 0.2180 mmol) were combined and stirred in dichloromethane (0.5 mL) for 30 minutes. Sodium triacetoxyborohydride (77 mg, 0.3633 mmol) was added under nitrogen gas, and the mixture was stirred for another 30 minutes. More sodium triacetoxyborohydride (154 mg, 0.7266 mmol) was added, and the final reaction mixture was stirred for 1 hour. The product was purified by reverse-phase HPLC eluting with a 10-99% acetonitrile/water gradient over 15 minutes with 5 mM acid modifier in the aqueous phase to give 3-[[4-(2,6-dimethylphenyl)-6-[(2R)-2-[(6,6-dimethyltetrahydropyran-2-yl)methylamino]-4,4-dimethyl-pentoxy]pyrimidin-2-yl]sulfamoyl]benzoic acid (hydrochloride salt) (57 mg, 46%) as a white solid. ESI-MS m/z calc. 638.3138, found 639.4 (M+1)⁺; Retention time: 1.41 minutes. LC method A.

### Step 5: (11R)-12-[(6,6-Dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione, diastereomer 1 (Compound 19), and (11R)-12-[(6,6-Dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione, diastereomer 2 (Compound 20)

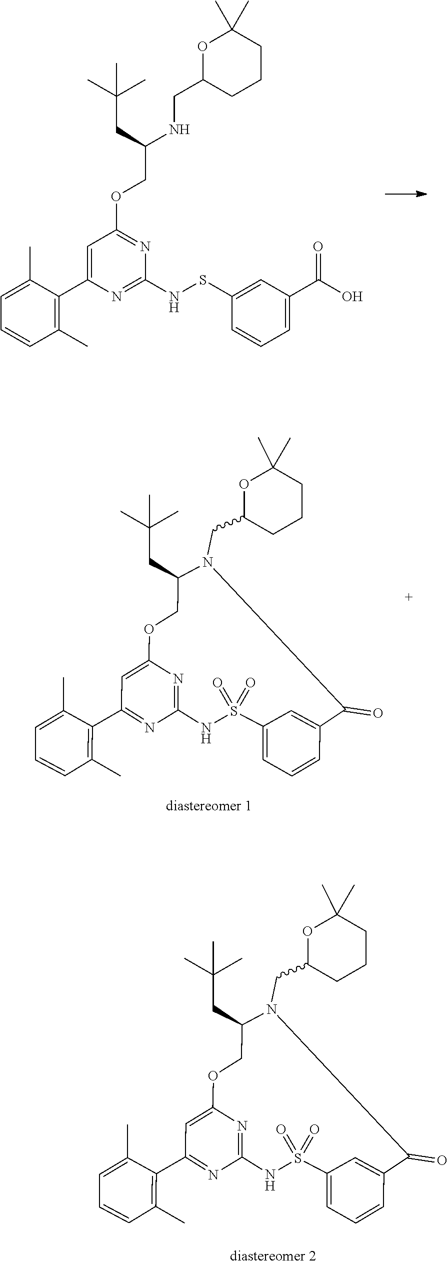 [SMILES: Cc1cccc(C)c1-c1cc(OC[C@@H](CC(C)(C)C)NCC2CCCC(C)(C)O2)nc(NSc2cccc(C(=O)O)c2)n1 ; Cc1cccc(C)c1-c1cc2nc(n1)NS(=O)(=O)c1cccc(c1)C(=O)N(CC1CCCC(C)(C)O1)[C@H](CC(C)(C)C)CO2 ; Cc1cccc(C)c1-c1cc2nc(n1)NS(=O)(=O)c1cccc(c1)C(=O)N(CC1CCCC(C)(C)O1)[C@H](CC(C)(C)C)CO2]

3-[[4-(2,6-dimethylphenyl)-6-[(2R)-2-[(6,6-dimethyltetrahydropyran-2yl)methylamino]-4,4-dimethyl-pentoxy]pyrimidin-2-yl]sulfamoyl]benzoic acid (hydrochloride salt) (57 mg, 0.08441 mmol) and 2-chloro-4,6-dimethoxy-1,3,5-triazine (22 mg, 0.1253 mmol) were combined and dissolved in DMF (1.7 mL). N-Methylmorpholine (43 mg, 0.4251 mmol) was added at 0° C. The reaction mixture was allowed to stir overnight at room temperature. The product was isolated by reverse-phase HPLC eluting with a 10-99% acetonitrile/water gradient over 15 minutes with 5 mM acid modifier in the aqueous phase to give two isomers: (11R)-12-[(6,6-dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (5.6 mg, 21%) diastereomer 1 was obtained as a white solid. ESI-MS m/z calc. 620.3032, found 621.3 (M+1)⁺; Retention time: 2.12 minutes, LC method A. (11R)-12-[(6,6-dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (1.7 mg, 6%) diastereomer 2 was obtained as a white solid. ESI-MS m/z calc. 620.3032, found 621.4 (M+1)⁺; Retention time: 2.19 minutes. LC method A.

### Example 15: Preparation of Compound 21

### Step 1: 2,2-Dimethylpent-4-en-1-ol

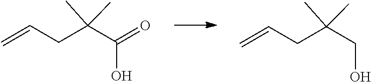

To a solution of 2,2-dimethylpent-4-enoic acid (5 g, 39.011 mmol) in THE (100 mL) cooled to 0° C. was added dropwise lithium aluminum hydride solution in THF (43 mL of 1 M, 43.000 mmol) over 30 minutes and the reaction mixture then stirred overnight with slow warming to room temperature. The mixture was cooled down to 0° C. and quenched by slowly adding water (2.6 mL), NaOH aqueous solution (5.2 mL; 15% w/w) and water (2.6 mL). The precipitate was removed by filtration; the cake was washed with DCM (75 mL). The filtrate was dried over sodium sulfate, filtered and concentrate under reduced pressure to afford 2,2-dimethylpent-4-en-1-ol (7.9 g, 110%) as a pale-yellow oil. ESI-MS m/z calc. 114.10446, found 115.2 (M+1)⁺; Retention time: 0.99 minutes (LC method Y).

### Step 2: (4,4-Dimethyltetrahydrofuran-2-yl)methanol

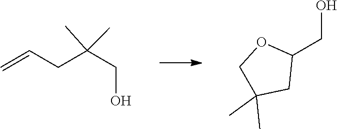

To a solution of 2,2-dimethylpent-4-en-1-ol (5.18 g, 45.365 mmol) in dichloromethane (100 mL) was added 3-chloroperoxybenzoic acid (12 g, 51.459 mmol) in portions over 10 minutes. The mixture was stirred over 90 h at room temperature. The mixture was dried over sodium sulfate, filtered and concentrated. Pentane (50 mL) was added and the precipitate was removed. The crude was purified by flash chromatography (100 g silica) using diethyl ether (0 to 100%) in pentane to give (4,4-dimethyltetrahydrofuran-2-yl)methanol (4.19 g, 71%) as a colorless oil. ESI-MS m/z calc. 130.09938, found 131.4 (M+1)⁺; Retention time: 1.28 minutes; LC method X.

### Step 3: 4,4-Dimethyltetrahydrofuran-2-carbaldehyde

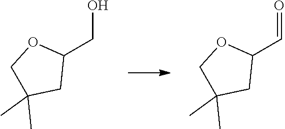

A buffered solution of bleach was prepared by dissolving sodium bicarbonate (123 mg, 1.4642 mmol) in sodium hypochlorite aqueous solution (1.7 mL of 1.505 M, 2.5585 mmol). That solution was then added dropwise to a second solution, that was prepared in advance by adding sodium bromide (13 mg, 0.1263 mmol) (dissolved in water (0.1 mL)) and TEMPO (1.5 mg, 0.0096 mmol) to (4,4-dimethyltetrahydrofuran-2-yl)methanol (300 mg, 2.1892 mmol) in dichloromethane (4 mL) maintained at ˜12° C. During the addition of the buffered bleach solution to the substrate solution the internal temperature was maintained below −10° C. After completion of the addition, the reaction mixture was maintained between −12° C. and −10° C. for 15 min. The excess bleach was quenched with ethanol (55.230 mg, 0.07 mL, 1.1989 mmol). The mixture was partitioned between dichloromethane (10 mL) and water (10 mL). The aqueous phase was separated and extracted with dichloromethane (2×10 mL). The organics were combined, washed with brine (2×10 mL), dried with sodium sulfate, filtered and concentrated under reduced pressure. The resulting oil was purified by flash chromatography on silica gel (SNAP 25 g column) using a gradient of 0% to 50% of diethyl ether in pentane to give 4,4-dimethyltetrahydrofuran-2-carbaldehyde (49.9 mg, 14%) as a pale-yellow oil. ¹H NMR (400 MHz, CDCl₃) δ 9.73 (d, J 1.7 Hz, 1H), 4.41-4.35 (m, 1H), 3.66-3.60 (m, 2H), 2.02-1.95 (m, 1H), 1.78 (dd, J 12.7, 7.3 Hz, 1H), 1.14 (s, 3H), 1.09 (s, 3H). GC-FID (GC method 1B): Retention time: 2.32 minutes.

### Step 4: 3-[[4-(2,6-Dimethylphenyl)-6-[(2R)-2-[(4,4-dimethyltetrahydrofuran-2-yl)methylamino]-4,4-dimethyl-pentoxy]pyrimidin-2-yl]sulfamoyl]benzoic acid

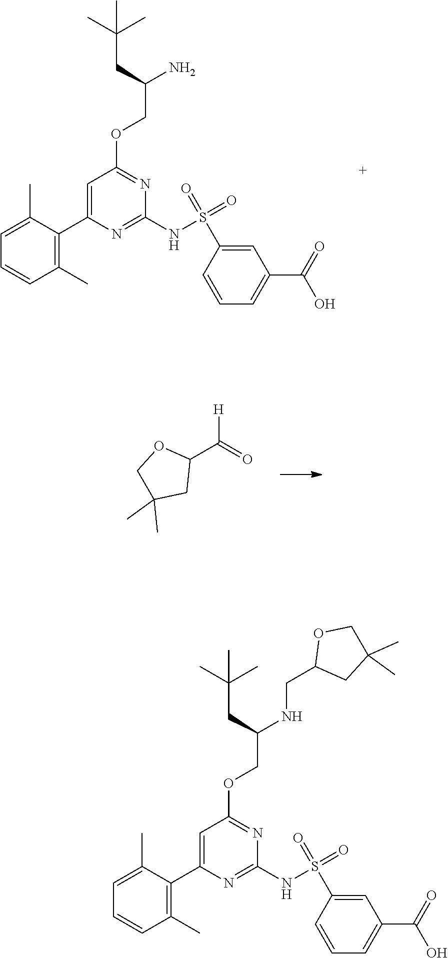

3-[[4-[(2R)-2-amino-4,4-dimethyl-pentoxy]-6-(2,6-dimethylphenyl)pyrimidin-2-yl]sulfamoyl]benzoic acid (hydrochloride salt) (103.6 mg, 0.1821 mmol) and 4,4-dimethyltetrahydrofuran-2-carbaldehyde (28 mg, 0.2185 mmol) were combined and stirred in dichloromethane (0.5 mL) for 30 minutes. Sodium triacetoxyborohydride (77 mg, 0.3633 mmol) was added under nitrogen gas, and the mixture was stirred for another 30 minutes. More sodium triacetoxyborohydride (154 mg, 0.7266 mmol) was added, and the final reaction mixture was stirred for 1 hour. The product was isolated by reverse-phase HPLC eluting with a 10-99% acetonitrile/water gradient over 15 minutes with 5 mM acid modifier in the aqueous phase. 3-[[4-(2,6-dimethylphenyl)-6-[(2R)-2-[(4,4-dimethyltetrahydrofuran-2-yl)methylamino]-4,4-dimethyl-pentoxy]pyrimidin-2-yl]sulfamoyl]benzoic acid (hydrochloride salt) (53 mg, 44%) was obtained as a white solid. ESI-MS m/z calc. 624.29816, found 625.3 (M+1)⁺; Retention time: 1.33 minutes. LC method A.

### Step 5: (11R)-12-[(4,4-Dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (Compound 21)

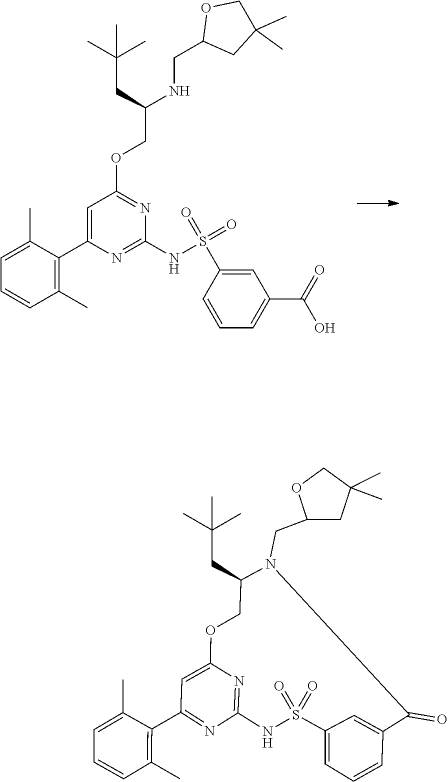 [SMILES: Cc1cccc(C)c1-c1cc(OC[C@@H](CC(C)(C)C)NCC2CC(C)(C)CO2)nc(NS(=O)(=O)c2cccc(C(=O)O)c2)n1 ; Cc1cccc(C)c1-c1cc2nc(n1)NS(=O)(=O)c1cccc(c1)C(=O)N(CC1CC(C)(C)CO1)[C@H](CC(C)(C)C)CO2]

3-[[4-(2,6-dimethylphenyl)-6-[(2R)-2-[(4,4-dimethyltetrahydrofuran-2-yl)methylamino]-4,4-dimethyl-pentoxy]pyrimidin-2-yl]sulfamoyl]benzoic acid (hydrochloride salt) (53 mg, 0.08015 mmol) and 2-chloro-4,6-dimethoxy-1,3,5-triazine (21 mg, 0.1196 mmol) were combined and dissolved in DMF (1.6 mL). N-methylmorpholine (41 mg, 0.4054 mmol) was added at 0° C. The reaction mixture was allowed to stir overnight at room temperature. After filtration, the product was isolated by reverse-phase HPLC eluting with a 30-60% acetonitrile/water gradient over 30 minutes with 5 mM acid modifier in the aqueous phase. (11R)-12-[(4,4-dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (1.9 mg, 8%) was obtained as a white solid. ESI-MS m/z calc. 606.2876, found 607.4 (M+1)⁺; Retention time: 2.04 minutes. LC method A

### Example 16: Preparation of Compound 22 and Compound 23

### Step 1: Ethyl 2,2-dimethylhex-5-enoate

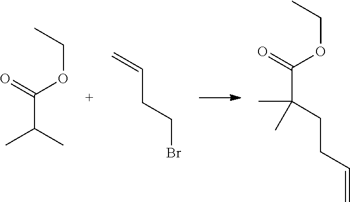

A solution of diisopropylamine (10.469 g, 14.5 mL, 103.46 mmol) in THE (40 mL) was cooled to −20° C. Hexyllithium (in hexanes) (41.1 mL of 2.3 M, 94.530 mmol) was added over 15 min while keeping the temperature between −20° C. and −10° C. and the reaction was stirred for 15 min. Ethyl isobutyrate (10 g, 11.561 mL, 86.090 mmol) was added over 15 min while keeping the temperature between −20° C. and −10° C. and the reaction was stirred for 15 min. DMPU (11.066 g, 10.4 mL, 86.337 mmol) was added over 15 min while keeping the temperature between −20° C. and −10° C. and the reaction was stirred for 15 min. 4-Bromobut-1-ene (12.768 g, 9.6 mL, 94.576 mmol) was added over 15 min while keeping the temperature between −20° C. and −10° C. and the reaction was stirred for 15 min. The reaction mixture was then stirred at room temperature for 1 h. 1 N aqueous HCl (100 mL) and diethyl ether (50 mL) were added. The phases were separated and the aqueous one was extracted with diethyl ether (3×30 mL). The combined organic layers were washed with water (3×100 mL), brine (50 mL), dried over sodium sulfate, filtered and concentrated under reduced pressure. Afforded ethyl 2,2-dimethylhex-5-enoate (24.15 g, 77%) as a yellow oil. ¹H NMR (400 MHz, CDCl₃) δ 5.84-5.73 (m, 1H), 5.00 (dq, J 17.2, 1.5 Hz, 1H), 4.95-4.90 (m, 1H), 4.11 (q, J 7.1 Hz, 2H), 2.04-1.95 (m, 2H), 1.65-1.59 (m, 2H), 1.25 (t, J=7.1 Hz, 3H), 1.18 (s, 6H). ESI-MS m/z calc. 170.13068, found 170.4 (M+1)⁺; Retention time: 1.65 minutes; LC method X.

### Step 2: 2,2-Dimethylhex-5-en-1-ol

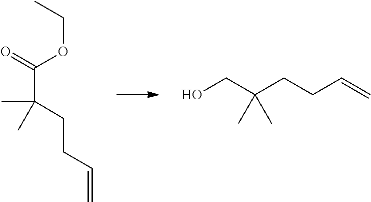

To a suspension of lithium aluminum hydride (4 g, 105.39 mmol) in THE (120 mL) cooled at 0° C. was added a solution of ethyl 2,2-dimethylhex-5-enoate (14.6 g, 85.757 mmol) in THE (70 mL) over 20 min. The reaction mixture was stirred at 0° C. for 1 h then at room temperature. After 2 h the reaction mixture was cooled down to 0° C., diluted with ether (100 mL), and water (4 mL) was added dropwise. Aqueous NaOH (2 N, 4 mL) was subsequently added followed by water (12 mL). The reaction mixture was then stirred at room temperature and sodium sulfate (5 g) was added. After 2 h, the mixture was filtered, rinsing the cake with diethyl ether (2×80 mL). The filtrate was concentrated under reduced pressure. Afforded 2,2-dimethylhex-5-en-1-ol (12.6 g, 68%) as a light-yellow oil that contains 41 mol % of THF. ¹H NMR (400 MHz, CDCl₃) δ 5.88-5.78 (m, 1H), 5.05-4.99 (m, 1H), 4.95-4.91 (m, 1H), 3.34-3.32 (d, J 4.2 Hz, 2H), 2.06-1.99 (m, 2H), 1.36-1.32 (m, 3H), 0.89 (s, 6H). ESI-MS m z calc. 128.12012, found 129.4 (M+1)⁺; Retention time: 1.68 minutes; LC method X.

### Step 3: (5,5-Dimethyltetrahydropyran-2-yl)methanol

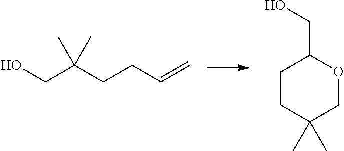

To a solution of 2,2-dimethylhex-5-en-1-ol (11.6 g, 53.380 mmol) in dichloromethane (120 mL) at 0° C. was added 3-chloroperoxybenzoic acid (13.7 g, 58.749 mmol) and sodium sulfate (3 g). The reaction was stirred 16 h at room temperature. More 3-chloroperoxybenzoic acid (2 g, 8.5764 mmol) was added and the reaction was stirred at room temperature for 20 h. The reaction was filtered, and the cake was washed with pentane (40 mL). The filtrate was concentrated under reduced pressure and the yielded white solid was washed with pentane (100 mL). The filtrate was concentrated under reduced pressure and the resulting oil was diluted in DCM (100 mL). The afforded solution was washed with saturated aqueous sodium bicarbonate (4×20 mL). The organic layer was washed with brine (50 mL), dried over sodium sulfate, filtered and concentrated under reduced pressure. The crude residue was purified by flash-chromatography on a 120 g silica gel cartridge, using a gradient of EtOAc in Heptanes (5 to 30% in 15 CV). Afforded after removal of the volatiles 2,2-dimethyl-4-(oxiran-2-yl)butan-1-ol (1.5 g, 17%) as a light yellow oil. ¹H NMR (400 MHz, CDCl₃) δ 3.33 (d, J 0.7 Hz, 2H), 2.96-2.88 (m, 1H), 2.76 (t, J 4.5 Hz, 1H), 2.49 (dd, J 5.0, 2.8 Hz, 1H), 1.60-1.32 (m, 5H), 0.89 (s, 6H). ESI-MS m/z calc. 144.115, found 145.2 (M+1)⁺; Retention time: 1.36 minutes (LC method X). and (5,5-dimethyltetrahydropyran-2-yl)methanol (4.1 g, 38%) as a colorless oil. ¹H NMR (400 MHz, CDCl₃) δ 3.70-3.46 (m, 3H), 3.41-3.30 (m, 1H), 3.21-3.16 (m, 1H), 2.04 (br s, 1H), 1.63-1.46 (m, 2H), 1.44-1.31 (m, 2H), 1.02 (s, 3H), 0.83 (s, 3H). ESI-MS m/z calc. 144.11504, found 145.4 (M+1)⁺; Retention time: 1.44 minutes; LC method X.

### Step 4: 5,5-Dimethyltetrahydropyran-2-carbaldehyde

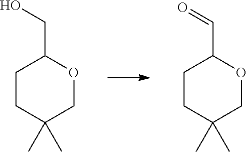

To a 0° C. solution of (5,5-dimethyltetrahydropyran-2-yl)methanol (201 mg, 1.3938 mmol) in water saturated DCM (10 mL) was added Dess-Martin periodinane (600 mg, 1.4146 mmol) and the reaction was stirred for 30 min at room temperature. A mixture of aqueous saturated solutions of sodium thiosulfate (5 mL), saturated sodium bicarbonate (5 mL), water (2 mL) and 1N NaOH (4 mL, to reach pH=9) was added and the reaction mixture was stirred for 5 min. The phases were separated and the aqueous one was extracted with DCM (3×10 mL). The combined organic layers were dried over sodium sulfate, filtered and concentrated under reduced pressure. The crude residue was purified by flash-chromatography on a 12 g silica gel cartridge, using a gradient of diethyl ether in pentane of 5 to 25% in 15 CV. Afforded 5,5-dimethyltetrahydropyran-2-carbaldehyde (108 mg, 38%) as a colorless oil. ESI-MS m/z calc. 142.09938, found 143.1 (M+1)⁺; Retention time: 2.43 minutes; LC method Y.

### Step 5: 3-[[4-(2,6-Dimethylphenyl)-6-[(2R)-2-[(5,5-dimethyltetrahydropyran-2-yl)methylamino]-4,4-dimethyl-pentoxy]pyrimidin-2-yl]sulfamoyl]benzoic acid

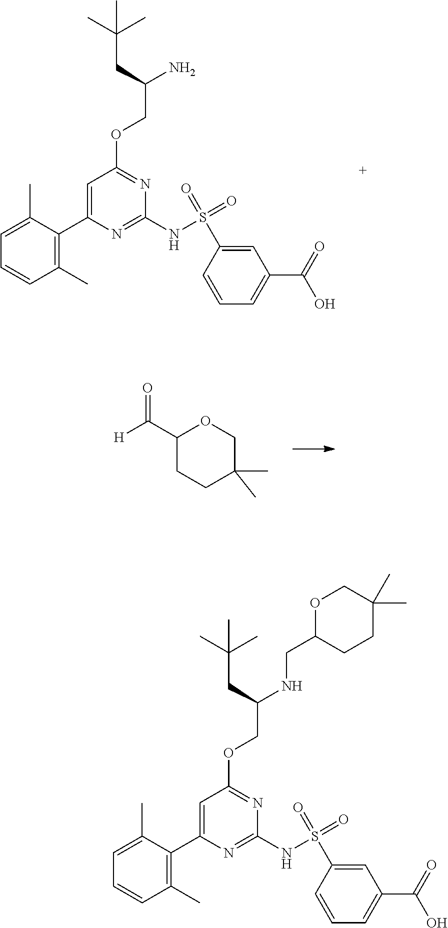

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (200 mg, 0.3642 mmol) in dichloromethane (2.5 mL) was added a solution of 5,5-dimethyltetrahydropyran-2-carbaldehyde (90 mg, 0.4386 mmol) in dichloromethane (2.5 mL) and the reaction was stirred at room temperature for 0.5 h. Sodium triacetoxyborohydride (386 mg, 1.8213 mmol) was added and the reaction was stirred at room temperature for 1.5 h. 1 N aqueous HCl was added (10 mL) and the phases were separated. The aqueous layer was extracted with EtOAc (3×10 mL). The combined organic layers were dried over sodium sulfate, filtered and concentrated under reduced pressure. The residue was evaporated with heptanes (2×50 mL). Afforded 3-[[4-(2,6-dimethylphenyl)-6-[(2R)-2-[(5,5-dimethyltetrahydropyran-2-yl)methylamino]-4,4-dimethyl-pentoxy]pyrimidin-2-yl]sulfamoyl]benzoic acid (250 mg, 51%) as a yellow semi-solid ESI-MS m/z calc. 638.3138, found 639.4 (M+1)⁺; Retention time: 1.55 minutes. LC method X.

### Step 6: (11R)-6-(2,6-Dimethylphenyl)-11-(2,2-dimethylpropyl)-12-[(5,5-dimethyltetrahydropyran-2-yl)methyl]-2,2-dioxo-9-oxa-2⁶-thia-3,5,12,19-tetrazatricyclo[12.3.1.14,8]nonadeca-1(18),4(19),5,7,14,16-hexaen-13-one

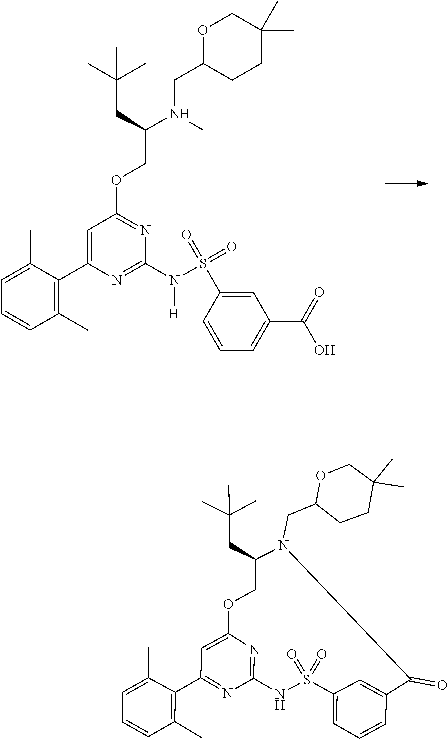 [SMILES: Cc1cccc(C)c1-c1cc(OC[C@@H](CC(C)(C)C)N(C)CC2CCC(C)(C)CO2)nc(NS(=O)(=O)c2cccc(C(=O)O)c2)n1 ; Cc1cccc(C)c1-c1cc2nc(n1)NS(=O)(=O)c1cccc(c1)C(=O)N(CC1CCC(C)(C)CO1)[C@H](CC(C)(C)C)CO2]

To a 0° C. solution of N-methylmorpholine (165.60 mg, 180 μL, 1.6372 mmol) in DMF (30 mL) was added 2-chloro-4,6-dimethoxy-1,3,5-triazine (90 mg, 0.5126 mmol) followed by 3-[[4-(2,6-dimethylphenyl)-6-[(2R)-2-[(5,5-dimethyltetrahydropyran-2-yl)methylamino]-4,4-dimethyl-pentoxy]pyrimidin-2-yl]sulfamoyl]benzoic acid (hydrochloride salt) (232 mg, 0.3291 mmol). After 5 min the reaction was stirred at room temperature for 36 h and then at 50° C. for 20 h. The reaction mixture was concentrated under reduced pressure at 50° C. The remaining crude was diluted with DCM (50 mL) and the solution was washed with a 1:1 v/v mix of water and brine (4×20 mL), water (25 mL) and brine (50 mL). The resulting organic layer was dried over sodium sulfate, filtered and concentrated under reduced pressure. The crude was purified by flash chromatography using a 24 g cartridge, eluting with a gradient of EtOAc in DCM (5 to 50% in 25 CV). Removal of the volatiles under reduced pressure afforded a white solid which was lyophilized and purified by reverse phase chromatography on a 15.5 g Cis cartridge using a gradient of MeCN in acidic water (0.1% v/v of formic acid in water) of 40 to 100% for 15 CV then 100% for 5 CV. The fractions containing the product were evaporated and then lyophilized. Afforded (11R)-6-(2,6-dimethylphenyl)-11-(2,2-dimethylpropyl)-12-[(5,5-dimethyltetrahydropyran-2-yl)methyl]-2,2-dioxo-9-oxa-2?⁶-thia-3,5,12,19-tetrazatricyclo[12.3.1.14,8]nonadeca-1(18),4(19),5,7,14,16-hexaen-13-one (17 mg, 8%) as a white solid. ESI-MS m/z calc. 620.3032, found 621.3 (M+1)⁺; Retention time: 5.0 minutes. ESI-MS m/z calc. 620.3032, found 621.3 (M+1)⁺; Retention time: 5.0 minutes; LC method Y; ¹H NMR (400 MHz, DMSO-d₆) δ 13.32-12.64 (m, 1H), 8.48 (br s, 0.6H), 8.43 (br s, 0.4H), 8.02-7.86 (m, 1H), 7.79-7.57 (m, 2H), 7.33-7.20 (m, 1H), 7.19-7.03 (m, 2H), 6.51-6.27 (m, 1H), 5.16-4.98 (m, 1H), 4.34-4.12 (m, 1H), 3.95-3.86 (m, 0.4H), 3.84-3.73 (m, 0.6H), 3.69-3.53 (m, 2H), 3.48-3.38 (m, 1H), 3.29-3.23 (m, 0.6H), 3.17-3.03 (m, 1.4H), 2.24-1.81 (m, 6H), 1.72-1.21 (m, 6H), 1.01 (s, 3H), 0.82-0.75 (m, 3H), 0.57-0.42 (m, 9H).

### Step 7: (11R)-12-[(5,5-Dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 22), and (11R)-12-[(5,5-dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 2 (Compound 23)

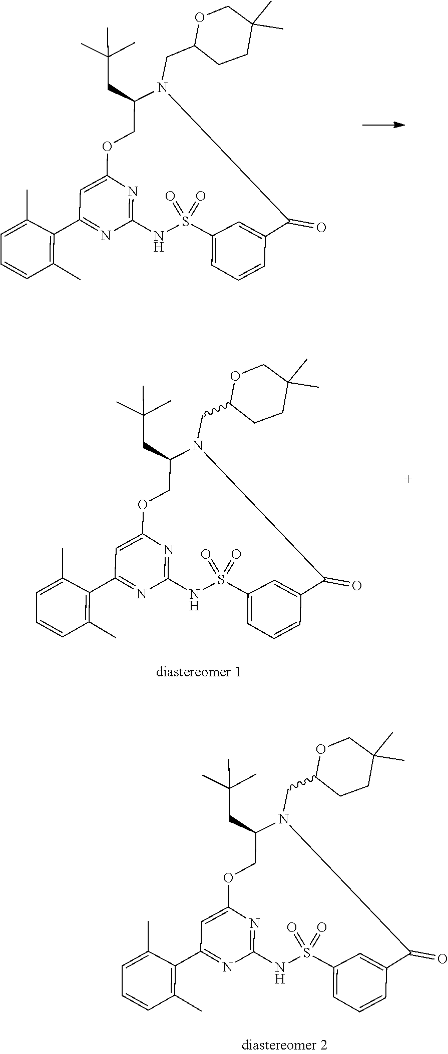 [SMILES: Cc1cccc(C)c1-c1cc2nc(n1)NS(=O)(=O)c1cccc(c1)C(=O)N(CC1CCC(C)(C)CO1)[C@H](CC(C)(C)C)CO2 ; Cc1cccc(C)c1-c1cc2nc(n1)NS(=O)(=O)c1cccc(c1)C(=O)N(CC1CCC(C)(C)CO1)[C@H](CC(C)(C)C)CO2 ; Cc1cccc(C)c1-c1cc2nc(n1)NS(=O)(=O)c1cccc(c1)C(=O)N(CC1CCC(C)(C)CO1)[C@H](CC(C)(C)C)CO2]

(11R)-12-[(5,5-Dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (15.3 mg, 0.02465 mmol) (mixture of diastereomers 60:40), was dissolved in DMSO (1 mL). Purification by reverse phase HPLC (10-80% over 25 min, then 80-99% over 3 min of acetonitrile in 5 mM HCl) provided diastereomer 1, (11R)-12-[(5,5-dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (7 mg, 76%). ESI-MS m/z calc. 620.3032, found 621.4 (M+1)⁺; Retention time: 2.19 minutes. LC method A. ¹H NMR (400 MHz, DMSO-d₆) δ 13.34-11.29 (m, 1H), 8.48 (s, 1H), 7.93 (s, 1H), 7.66 (s, 2H), 7.35-7.19 (m, 1H), 7.19-6.97 (m, 2H), 6.39 (s, 1H), 5.08 (dd, J 10.7, 4.4 Hz, 1H), 4.26 (t, J 10.8 Hz, 1H), 3.84-3.69 (m, 1H), 3.69-3.54 (m, 2H), 3.41 (dd, J 11.0, 2.2 Hz, 1H), 3.28 (d, J 9.1 Hz, 1H), 3.11 (d, J 11.0 Hz, 1H), 2.23-1.80 (m, 7H), 1.64-1.20 (m, 5H), 1.01 (s, 3H), 0.79 (s, 3H), 0.50 (s, 9H), and diastereomer 2, (11R)-12-[(5,5-dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (5.1 mg, 83%). ESI-MS m/z calc. 620.3032, found 621.51 (M+1)⁺; Retention time: 2.28 minutes. LC method A. ¹H NMR (400 MHz, DMSO-d₆) δ 13.34-11.29 (m, 1H), 8.48 (s, 1H), 7.93 (s, 1H), 7.66 (s, 2H), 7.35-7.19 (m, 1H), 7.19-6.97 (m, 2H), 6.39 (s, 1H), 5.08 (dd, J 10.7, 4.4 Hz, 1H), 4.26 (t, J 10.8 Hz, 1H), 3.84-3.69 (m, 1H), 3.69-3.54 (m, 2H), 3.41 (dd, J 11.0, 2.2 Hz, 1H), 3.28 (d, J 9.1 Hz, 1H), 3.11 (d, J 11.0 Hz, 1H), 2.23-1.80 (m, 7H), 1.64-1.20 (m, 5H), 1.01 (s, 3H), 0.79 (s, 3H), 0.50 (s, 9H).

### Example 17: Preparation of Compound 24 and Compound 25

### Step 1: Ethyl 3,3-dimethylhex-5-enoate

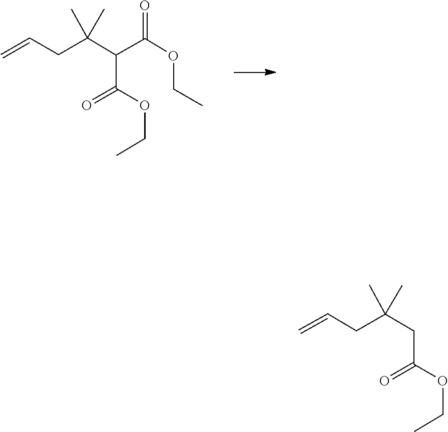 [SMILES: C=CCC(C)(C)C(C(=O)OCC)C(=O)OCC ; C=CCC(C)(C)CC(=O)OCC]

LiCl (3.15 g, 74.303 mmol) was added to a solution of diethyl 2-(1,1-dimethylbut-3-enyl)propanedioate (9.4 g, 38.793 mmol) in DMSO (50 mL) and water (1 mL) and heated at 160° C. for 48 h. The reaction was cooled and diluted with brine and diethyl ether. The aqueous layer was extracted with diethyl ether. The organic phase was combined and washed with saturated aqueous NaCl solution, dried over magnesium sulfate, filtered and evaporated to give a brown oil. The resulting crude product was purified by flash chromatography on a 80 g silica gel cartridge using a gradient of 0-30% EtOAc in heptanes to give ethyl 3,3-dimethylhex-5-enoate (5.75 g, 87%) as a colorless oil. ¹H NMR (400 MHz, CHLOROFORM-d) δ 5.88-5.76 (m, 1H), 5.12-4.98 (m, 2H), 4.12 (q, J 7.1 Hz, 2H), 2.18 (s, 2H), 2.08 (d, J 7.6 Hz, 2H), 1.25 (t, J 7.2 Hz, 3H), 1.00 (s, 6H). ESI-MS m/z calc. 170.13068, found 171.1 (M+1)⁺; Retention time: 1.96 minutes; LC method X.

### Step 2: 3,3-Dimethylhex-5-en-1-ol

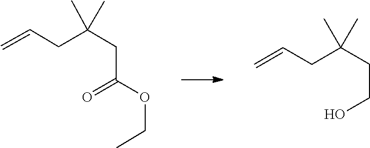

To a suspension of LAH (1.4 g, 36.886 mmol) in dry diethylether (30 mL) was added at 0° C. a solution of ethyl 3,3-dimethylhex-5-enoate (5.7 g, 33.48 mmol) in dry diethyl ether (10 mL). The mixture was stirred for 30 min at 0° C. and overnight at room temperature. Then, water (20 ml) and a solution of NaOH 1M (20 mL) was added carefully to the mixture and stirred 1 h. The resulting mixture was filtered through a Celite pad. The filtrate was concentrated and the resulting crude product was purified by flash-chromatography on a 80 g silica gel cartridge using a gradient of 0-30% EtOAc in heptanes to give 3,3-dimethylhex-5-en-1-ol (3.1 g, 66%) as a colorless oil. ¹H NMR (400 MHz, CHLOROFORM-d) δ 5.98-5.71 (m, 1H), 5.14-4.96 (m, 2H), 3.75-3.67 (m, 2H), 1.98 (d, J 7.6 Hz, 2H), 1.56-1.50 (m, 2H), 1.44-1.32 (m, 1H), 0.91 (s, 6H).

### Step 3: (4,4-Dimethyltetrahydropyran-2-yl)methanol

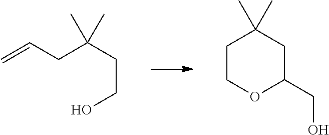

To a solution of 3,3-dimethylhex-5-en-1-ol (2.4 g, 18.719 mmol) in dichloromethane (40 mL) at 0° C. was added m-CPBA (5.23 g, 22.427 mmol). The reaction was stirred 16 h at room temperature. The reaction was filtered, and the cake was washed with DCM (20 mL). The filtrate was concentrated under reduced pressure and the yielded white solid was washed with pentane (100 mL). The filtrate was concentrated under reduced pressure and the resulting oil was diluted in DCM (100 mL). The afforded solution was washed with saturated aqueous sodium bicarbonate (4×20 mL). The organic layer was washed with brine (50 mL), dried over sodium sulfate, filtered and concentrated under reduced pressure. The crude was purified in a short plug of silica gel eluting with 30% of ethyl acetate in heptanes to give (4,4-Dimethyltetrahydropyran-2-yl)methanol (1.13 g, 36%). ¹H NMR (400 MHz, CHLOROFORM-d) δ 3.86 (ddd, J 11.7, 5.1, 1.3 Hz, 1H), 3.68-3.42 (m, 4H), 2.25-2.16 (m, 1H), 1.55-1.42 (m, 1H), 1.28-1.15 (m, 3H), 1.02 (s, 3H), 0.96 (s, 3H).

### Step 4: 4,4-Dimethyltetrahydropyran-2-carbaldehyde

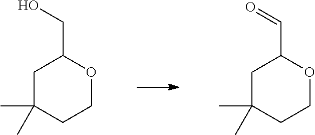

To a 0° C. solution of (4,4-dimethyltetrahydropyran-2-yl)methanol (1.135 g, 7.8704 mmol) in water-saturated DCM (10 mL) was added Dess-Martin periodinane (3.4 g, 8.0162 mmol) and the reaction was stirred for 30 min at room temperature. A mixture of aqueous saturated solutions of sodium thiosulfate (5 mL), saturated sodium bicarbonate (5 mL), water (2 mL) and 1N NaOH (4 mL, to reach pH=9) was added and the reaction mixture was stirred for 5 min. The phases were separated and the aqueous one was extracted with DCM (3×10 mL). The combined organic layers were dried over sodium sulfate, filtered and concentrated under reduced pressure. The crude was purified in a short plug of silica gel eluting with 30% of DCM in pentane to give 4,4-dimethyltetrahydropyran-2-carbaldehyde (850 mg, 49%) as a colorless oil.

### Step 5: 3-[[4-(2,6-Dimethylphenyl)-6-[(2R)-2-[(4,4-dimethyltetrahydropyran-2-yl)methylamino]-4,4-dimethyl-pentoxy]pyrimidin-2-yl]sulfamoyl]benzoic acid

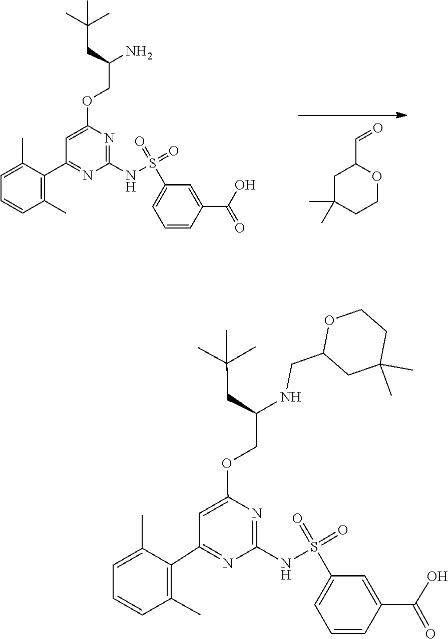

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (265 mg, 0.4826 mmol) in dichloromethane (8 mL) was added 4,4-dimethyltetrahydropyran-2-carbaldehyde (127 mg, 0.5805 mmol) and the reaction was stirred at room temperature for 0.5 h and sonicated during 5 min. Sodium triacetoxyborohydride (512 mg, 2.3433 mmol) was added and the reaction was stirred at room temperature for 1.5 h. Then, 1 N aqueous HCl was added (10 mL) and the phases were separated. The aqueous layer was extracted with EtOAc (3×10 mL). The combined organic layers were dried over sodium sulfate, filtered and concentrated under reduced pressure. The residue was evaporated with heptanes (2×25 mL) to remove residual acetic acid. Afforded 3-[[4-(2,6-dimethylphenyl)-6-[(2R)-2-[(4,4-dimethyltetrahydropyran-2-yl)methylamino]-4,4-dimethyl-pentoxy]pyrimidin-2-yl]sulfamoyl]benzoic acid (hydrochloride salt) (403 mg, 82%) as a pale-yellow semi-solid ESI-MS m/z calc. 638.3138, found 639.3 (M+1)⁺; Retention time: 2.82 minutes (LC method Y).

### Step 6: (11R)-6-(2,6-dimethylphenyl)-11-(2,2-dimethylpropyl)-12-[(4,4-dimethyltetrahydropyran-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 24), and (11R)-6-(2,6-dimethylphenyl)-11-(2,2-dimethylpropyl)-12-[(4,4-dimethyltetrahydropyran-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 25)

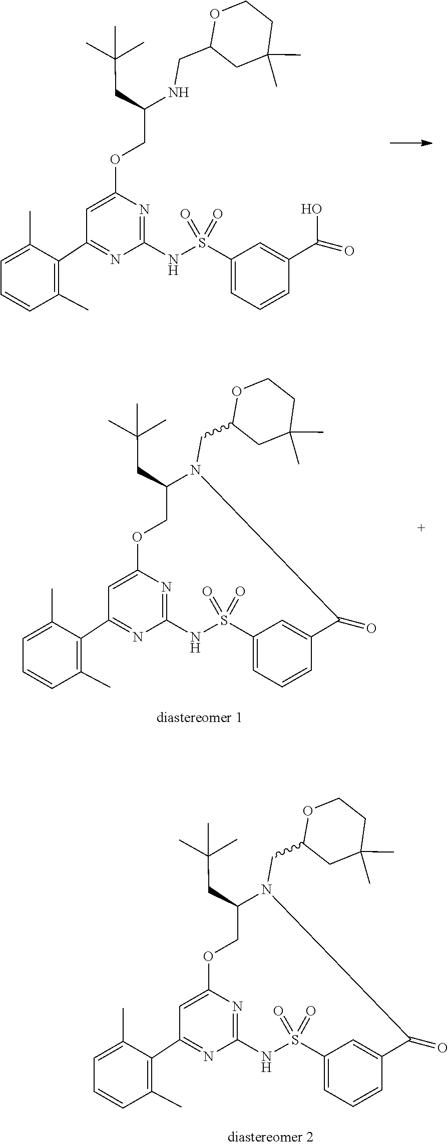 [SMILES: Cc1cccc(C)c1-c1cc(OC[C@@H](CC(C)(C)C)NCC2CC(C)(C)CCO2)nc(NS(=O)(=O)c2cccc(C(=O)O)c2)n1 ; Cc1cccc(C)c1-c1cc2nc(n1)NS(=O)(=O)c1cccc(c1)C(=O)N(CC1CC(C)(C)CCO1)[C@H](CC(C)(C)C)CO2 ; Cc1cccc(C)c1-c1cc2nc(n1)NS(=O)(=O)c1cccc(c1)C(=O)N(CC1CC(C)(C)CCO1)[C@H](CC(C)(C)C)CO2]

To a 0° C. solution of N-methylmorpholine (202 mg, 1.9971 mmol) in DMF (30 mL) was added 2-chloro-4,6-dimethoxy-1,3,5-triazine (105 mg, 0.5980 mmol) followed by 3-[[4-(2,6-dimethylphenyl)-6-[(2R)-2-[(4,4-dimethyltetrahydropyran-2-yl)methylamino]-4,4-dimethyl-pentoxy]pyrimidin-2-yl]sulfamoyl]benzoic acid (hydrochloride salt) (403 mg, 0.3975 mmol). After 5 min the reaction was stirred at room temperature for 16 h. The reaction mixture was concentrated under reduced pressure at 50° C. The remaining crude was diluted with DCM (50 mL) and the solution was washed with a 1:1 v/v mix of water and brine (4×20 mL), water (25 mL) and brine (50 mL). The resulting organic layer was dried over sodium sulfate, filtered and concentrated under reduced pressure. The crude was purified by flash chromatography on silica gel using a 40 g cartridge, eluting with a gradient of EtOAc in DCM (5 to 50%). Removal of the volatiles under reduced pressure afforded a white solid which was purified by reverse phase chromatography on a 50 g C₁₈ cartridge using a gradient of MeCN in acidic water (0.1% v/v of formic acid in water) of 5 to 100% for 20 CV. The fractions containing the product were evaporated and then lyophilized. Afforded two diastereomers as a white solid: (11R)-6-(2,6-dimethylphenyl)-11-(2,2-dimethylpropyl)-12-[(4,4-dimethyltetrahydropyran-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (57 mg, 22%) diastereomer 1, ¹H NMR (400 MHz, DMSO-d₆) δ 13.35-12.81 (m, 1H), 8.47 (br. s., 1H), 7.93 (br. s., 1H), 7.66 (br. s., 2H), 7.39-7.18 (m, 1H), 7.18-7.05 (m, 2H), 6.39 (br. s., 1H), 5.07 (dd, J=10.5, 4.2 Hz, 1H), 4.22 (t, J=11.1 Hz, 1H), 4.04-3.84 (m, 1H), 3.84-3.65 (m, 2H), 3.62-3.40 (m, 2H), 3.28-3.19 (m, 1H), 2.06-1.85 (m, 6H), 1.46-1.34 (m, 2H), 1.32-1.05 (m, 4H), 0.99 (s, 3H), 0.96 (s, 3H), 0.50 (s, 9H). ESI-MS m/z calc. 620.3032, found 621.3 (M+1)⁺; Retention time: 4.89 minutes; LC method Y; and (11R)-6-(2,6-dimethylphenyl)-11-(2,2-dimethylpropyl)-12-[(4,4-dimethyltetrahydropyran-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (35 mg, 14%) diastereomer 2, ¹H NMR (400 MHz, DMSO-d₆) δ 13.33-12.85 (m, 1H), 8.46 (br. s., 1H), 7.94 (br. s., 1H), 7.81-7.55 (m, 2H), 7.25 (d, J=7.3 Hz, 1H), 7.18-7.04 (m, 2H), 6.47-6.31 (m, 1H), 5.05 (d, J=7.8 Hz, 1H), 4.17 (t, J 11.2 Hz, 1H), 3.98-3.86 (m, 1H), 3.86-3.74 (m, 2H), 3.68-3.41 (m, 2H), 2.97 (dd, J 14.2, 8.8 Hz, 1H), 2.25-1.82 (m, 6H), 1.82-1.60 (m, 1H), 1.48-1.07 (m, 5H), 1.00 (s, 3H), 0.96 (s, 3H), 0.50 (s, 9H). ESI-MS m/z calc. 620.3032, found 621.3 (M+1)⁺; Retention time: 5.06 minutes; LC method Y.

### Example 18: Preparation of Compound 26 and Compound 27

### Step 1: 3,3-Dimethylpent-4-en-1-ol

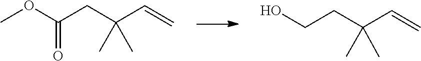

To a suspension of lithium aluminum hydride (3.2 g, 84.312 mmol) in THE (120 mL) cooled at 0° C. was added a solution of methyl 3,3-dimethylpent-4-enoate (9.8674 g, 11.2 mL, 68.005 mmol) in THE (70 mL) over 20 min. The reaction mixture was stirred at 0° C. for 1 h then at room temperature for 1 h. The reaction mixture was then cooled down to 0° C., diluted with ether (150 mL), and water (5 mL) was added dropwise. Aqueous NaOH (1 N, 10 mL) was subsequently added followed by water (15 mL). The reaction mixture was then stirred at room temperature and sodium sulfate (20 g) was added. After overnight stirring, the mixture was filtered, rinsing the cake with diethyl ether (3×100 mL). The filtrate was dried over sodium sulfate, filtered and concentrated under reduced pressure to furnish 3,3-dimethylpent-4-en-1-ol (7.76 g, 95%) as a translucent oil. ¹H NMR (400 MHz, CDCl₃) δ 5.93-5.74 (m, 1H), 5.03-4.84 (m, 2H), 3.62 (br t, J 7.2 Hz, 2H), 1.82-1.68 (m, 1H), 1.60 (t, J 7.2 Hz, 2H), 1.17-0.86 (s, 6H). ESI-MS m/z calc. 114.10446, found 115.1 (M+1)⁺; Retention time: 1.21 minutes; (LC method 1C).

### Step 2: (3,3-Dimethyltetrahydrofuran-2-yl)methanol

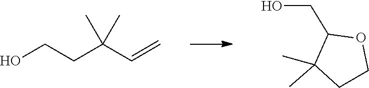

To a solution of 3,3-dimethylpent-4-en-1-ol (9.08 g, 79.520 mmol) in dichloromethane (140 mL) at 0° C. was added 3-Chloroperoxybenzoic acid (37 g, 158.66 mmol) and sodium sulfate (20 g). The reaction was allowed to warm to room temperature and stirred overnight, after which time all the starting material was consumed (TLC, heptane/EtOAc 7/3, v/v). Potassium carbonate (22 g, 159.18 mmol) was added and the reaction mixture was stirred at room temperature for an additional 30 minutes. The reaction was then filtered and concentrated under reduced pressure. diethyl ether (150 mL) was added and the organic layer was washed with saturated aqueous sodium bicarbonate (3×50 mL), with aqueous NaOH (1N, 25 mL), brine (50 mL), dried over sodium sulfate and concentrated under reduced pressure to furnish (3,3-dimethyltetrahydrofuran-2-yl)methanol (2.79 g, 22%) as a translucent oil.

### Step 3: 3,3-Dimethyltetrahydrofuran-2-carbaldehyde

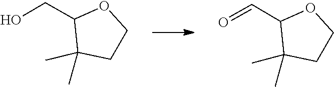

A solution of oxalyl chloride (552.90 mg, 380 μL, 4.3561 mmol) in dry dichloromethane (20.800 mL) was cooled down to −78° C. under nitrogen atmosphere. Dimethylsulfoxide (660.60 mg, 600 μL, 8.4547 mmol) was then added, followed by (3,3-dimethyltetrahydrofuran-2-yl)methanol (520 mg, 3.9943 mmol). The reaction was stirred at −78° C. for 30 min after which time triethylamine (2.0328 g, 2.8 mL, 20.089 mmol) was added dropwise and the reaction was allowed to warm up to room temperature for 1.5 h. A saturated ammonium chloride solution was added (20 mL) and the layers were separated. The aqueous layer was extracted with DCM (3×15 mL) and the combined organic layers were washed with brine (50 mL), dried over sodium sulfate, filtered and concentrated under reduced pressure (water bath at room temperature) to furnish 3,3-dimethyltetrahydrofuran-2-carbaldehyde (1.753 g, 82%) as a pale-yellow oil which was used in the next step without further purification.

### Step 4: 3-[[4-(2,6-dimethylphenyl)-6-[(2R)-2-[(3,3-dimethyltetrahydrofuran-2-yl)methylamino]-4,4-dimethyl-pentoxy]pyrimidin-2-yl]sulfamoyl]benzoic acid

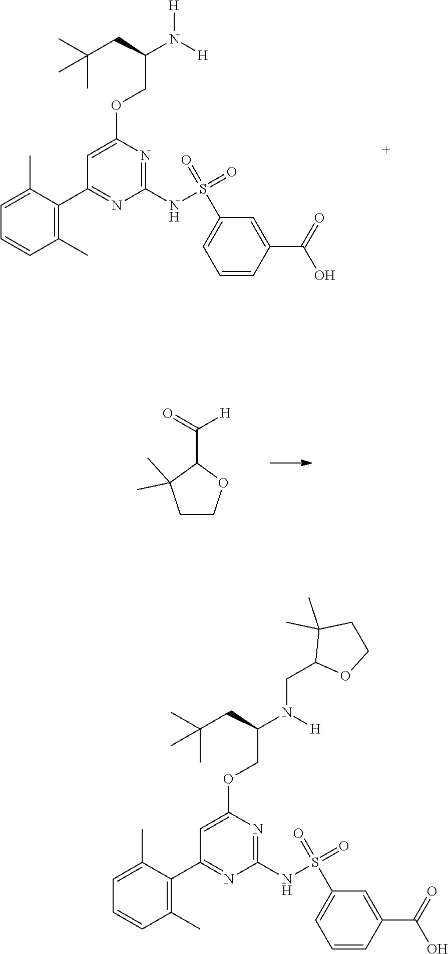

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (210 mg, 0.3825 mmol) in dichloromethane (2.5 mL) was added a solution of 3,3-dimethyltetrahydrofuran-2-carbaldehyde (245 mg, 0.4588 mmol) in dichloromethane (2.5 mL) and the reaction was stirred at room temperature for 0.5 h. Sodium triacetoxyborohydride (420 mg, 1.9222 mmol) was added and the reaction was stirred at room temperature for 1.5 h. 1 N aqueous HCl was added (10 mL) and the phases were separated. The aqueous layer was extracted with EtOAc (3×10 mL). The combined organic layers were dried over sodium sulfate, filtered and concentrated under reduced pressure. The residue was co-evaporated with heptanes (2×50 mL). Afforded 3-[[4-(2,6-dimethylphenyl)-6-[(2R)-2-[(3,3-dimethyltetrahydrofuran-2-yl)methylamino]-4,4-dimethyl-pentoxy]pyrimidin-2-yl]sulfamoyl]benzoic acid (hydrochloride salt) (267 mg, 64%) as a white foam ESI-MS m/z calc. 624.2982, found 625.3 (M+1)⁺; Retention time: 1.51 minutes; LC method X.

### Step 5: (11R)-6-(2,6-Dimethylphenyl)-11-(2,2-dimethylpropyl)-12-[(3,3-dimethyltetrahydrofuran-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

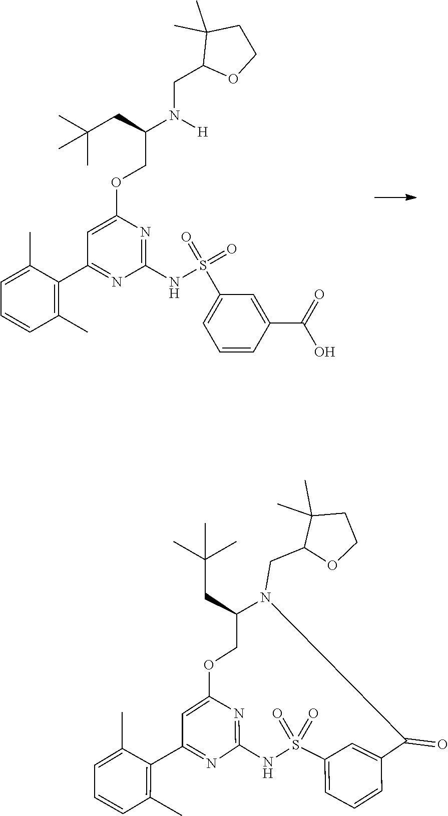 [SMILES: Cc1cccc(C)c1-c1cc(OC[C@@H](CC(C)(C)C)NCC2OCCC2(C)C)nc(NS(=O)(=O)c2cccc(C(=O)O)c2)n1 ; Cc1cccc(C)c1-c1cc2nc(n1)NS(=O)(=O)c1cccc(c1)C(=O)N(CC1OCCC1(C)C)[C@H](CC(C)(C)C)CO2]

To a 0° C. solution of N-methylmorpholine (130 mg, 0.1413 mL, 1.2724 mmol) in DMF (32. mL) was added 2-chloro-4,6-dimethoxy-1,3,5-triazine (90 mg, 0.5024 mmol) followed by 3-[[4-(2,6-dimethylphenyl)-6-[(2R)-2-[(3,3-dimethyltetrahydrofuran-2-yl)methylamino]-4,4-dimethyl-pentoxy]pyrimidin-2-yl]sulfamoyl]benzoic acid (hydrochloride salt) (267 mg, 0.2463 mmol). After 5 min the reaction was stirred at room temperature for 96 h. The reaction mixture then was concentrated under reduced pressure at 50° C., and the remaining crude was diluted with DCM (50 mL). The organic solution was washed with a 1:1 v/v mix of water and brine (4×20 mL), water (25 mL) and brine (50 mL). The organic layer was dried over sodium sulfate, filtered and concentrated under reduced pressure. The crude yellow residue was purified by flash chromatography using a 24 g cartridge, eluting with a gradient of EtOAc in DCM (10 to 50% in 20 CV then 50% for 2CV). Removal of the volatiles under reduced pressure afforded a white sticky solid which was purified by reverse phase chromatography on a 15.5 g Cis cartridge using a gradient of MeCN in acidic water (0.1% v/v of formic acid in water, 40 to 100% MeCN for 20 CV then 100% MeCN for 5 CV). The fractions containing the product were evaporated and then lyophilized to afford (11R)-6-(2,6-dimethylphenyl)-11-(2,2-dimethylpropyl)-12-[(3,3-dimethyltetrahydrofuran-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (31.6 mg, 21%) as a white fluffy solid. ESI-MS m/z calc. 606.2876, found 607.3 (M+1)⁺; Retention time: 4.79 minutes; LC method Y, ¹H NMR (400 MHz, DMSO-d₆) δ 13.64-11.60 (m, 1H), 8.58-8.36 (m, 1H), 8.02-7.82 (m, 1H), 7.79-7.54 (m, 2H), 7.35-7.19 (m, 1H), 7.19-7.04 (m, 2H), 6.52-6.28 (m, 1H), 5.17-5.01 (m, 1H), 4.29-4.14 (m, 1H), 3.97-3.84 (m, 1H), 3.84-3.76 (m, 1.5H), 3.76-3.65 (m, 2H), 3.59-3.51 (m, 0.5H), 3.39-3.33 (m, 0.5H), 2.95-2.77 (m, 0.5H), 2.25-1.87 (m, 6H), 1.81-1.72 (m, 2H), 1.72-1.64 (m, 0.5H), 1.37-1.30 (m, 0.5H), 1.29-1.21 (m, 1H), 1.17 (br s, 1.5H), 1.12 (br s, 1.5H), 1.02 (br s, 1.5H), 1.01 (br s, 1.5H), 0.57-0.44 (m, 9H).

### Step 6: (11R)-12-[(3,3-dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 26), and (11R)-12-[(3,3-dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 2 (Compound 27)

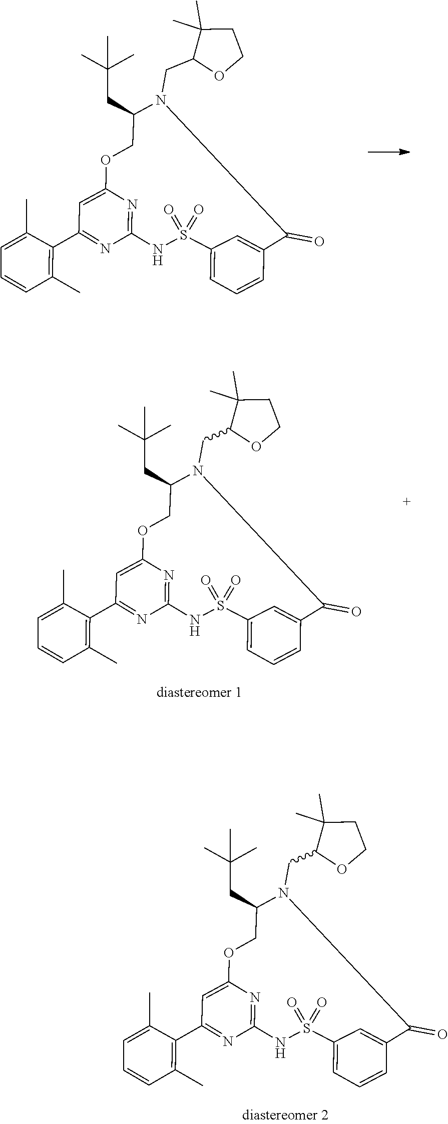 [SMILES: Cc1cccc(C)c1-c1cc2nc(n1)NS(=O)(=O)c1cccc(c1)C(=O)N(CC1OCCC1(C)C)[C@H](CC(C)(C)C)CO2 ; Cc1cccc(C)c1-c1cc2nc(n1)NS(=O)(=O)c1cccc(c1)C(=O)N(CC1OCCC1(C)C)[C@H](CC(C)(C)C)CO2 ; Cc1cccc(C)c1-c1cc2nc(n1)NS(=O)(=O)c1cccc(c1)C(=O)N(CC1OCCC1(C)C)[C@H](CC(C)(C)C)CO2]

(11R)-12-[(3,3-dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (28.9 mg, 0.04763 mmol) was dissolved in DMSO (1 mL). Purification by reverse phase HPLC (1-99% acetonitrile/5 mM HCl over 30 min) provided two separated diastereomers as a white solid: First to elute, more polar isomer: (11R)-12-[(3,3-dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione diastereomer 1 (13.5 mg, 93%). ESI-MS m/z calc. 606.2876, found 607.39 (M+1)⁺; Retention time: 2.06 minutes. LC method A, ¹H NMR (400 MHz, DMSO-d₆) δ 13.49-11.53 (broad m, 1H), 8.49 (s, 1H), 7.89 (br s, 1H), 7.77-7.44 (br m, 2H), 7.35-7.20 (m, 1H), 7.12 (s, 2H), 6.38 (br s, 1H), 5.09 (dd, J 10.9, 4.4 Hz, 1H), 4.21 (t, J 11.1 Hz, 1H), 3.80 (q, J 7.7 Hz, 1H), 3.76-3.63 (m, 3H), 3.55 (d, J 14.2 Hz, 1H), 3.39-3.35 (overlapped with water, m, 1H), 2.21 (dd, J 15.0, 8.3 Hz, 1H), 2.16-1.81 (br m, 6H), 1.75 (t, J 7.3 Hz, 2H), 1.26 (d, J=14.8 Hz, 1H), 1.17 (s, 3H), 1.01 (s, 3H), 0.50 (s, 9H); and a second to elute, less polar isomer: (11R)-12-[(3,3-dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione diastereomer 2 (10.2 mg, 67%). ESI-MS m z calc. 606.2876, found 607.58 (M+1)⁺; Retention time: 2.11 minutes. LC method A, ¹H NMR (400 MHz, DMSO-d₆) δ 13.46-11.22 (broad m, 1H), 8.43 (s, 1H), 7.94 (s, 1H), 7.68 (s, 2H), 7.26 (t, J 7.7 Hz, 1H), 7.13 (s, 2H), 6.39 (s, 1H), 5.09 (dd, J 11.7, 4.2 Hz, 1H), 4.21 (t, J 11.4 Hz, 1H), 3.94 (d, J=11.8 Hz, 1H), 3.87 (q, J=7.8 Hz, 1H), 3.83-3.75 (m, 2H), 3.70 (d, J 9.0 Hz, 1H), 2.86 (dd, J 14.0, 9.3 Hz, 1H), 2.24-1.85 (m, 6H), 1.82-1.74 (m, 2H), 1.68 (dd, J=15.3, 8.9 Hz, 1H), 1.34 (d, J=15.0 Hz, 1H), 1.12 (s, 3H), 1.02 (s, 3H), 0.51 (s, 9H).

### Example 19: Preparation of Compound 28 and Compound 29

### Step 1: 3-[[4-(2,6-Dimethylphenyl)-6-[(2R)-2-[(2,2-dimethyltetrahydropyran-4-yl)methylamino]-4,4-dimethyl-pentoxy]pyrimidin-2-yl]sulfamoyl]benzoic acid

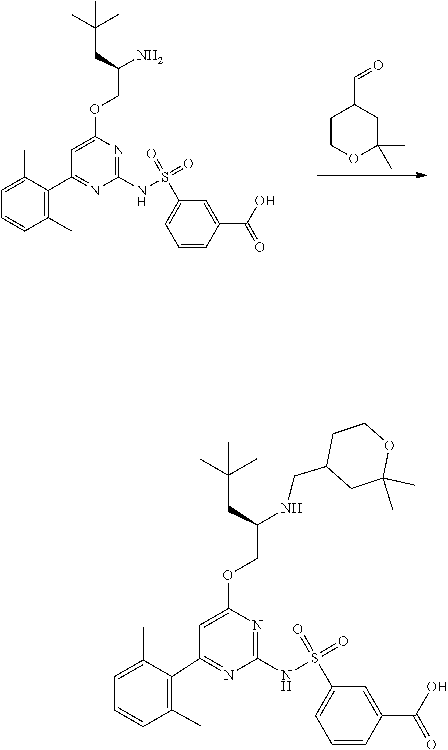

3-[[4-[(2R)-2-amino-4,4-dimethyl-pentoxy]-6-(2,6-dimethylphenyl)pyrimidin-2-yl]sulfamoyl]benzoic acid (hydrochloride salt) (100 mg, 0.1730 mmol) and 2,2-dimethyltetrahydropyran-4-carbaldehyde (30 mg, 0.2110 mmol) were combined and suspended in dichloromethane (0.5 mL). The mixture was stirred at room temperature for 30 minutes. Sodium triacetoxyborohydride (Sodium salt) (73 mg, 0.3444 mmol) was added, and the reaction mixture was stirred for 30 minutes before additional sodium triacetoxyborohydride (Sodium salt) (147 mg, 0.6936 mmol) was added. The final reaction mixture was stirred for 1 hour. Minimal aqueous 1 M HCl was added to quench the reaction. The product was isolated by UV-triggered reverse-phase HPLC eluting with a 10-99% acetonitrile/water gradient over 15 minutes with 5 mM acid modifier in the aqueous phase. 3-[[4-(2,6-dimethylphenyl)-6-[(2R)-2-[(2,2-dimethyltetrahydropyran-4-yl)methylamino]-4,4-dimethyl-pentoxy]pyrimidin-2-yl]sulfamoyl]benzoic acid (84 mg, 76%) was obtained as a white solid. ESI-MS m/z calc. 638.3138, found 639.4 (M+1)⁺; Retention time: 1.24 minutes; LC method A.

### Step 2: (11R)-12-[(2,2-Dimethyloxan-4-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione, diastereomer 1 (Compound 28), and (11R)-12-[(2,2-dimethyloxan-4-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione, diastereomer 2 (Compound 29)

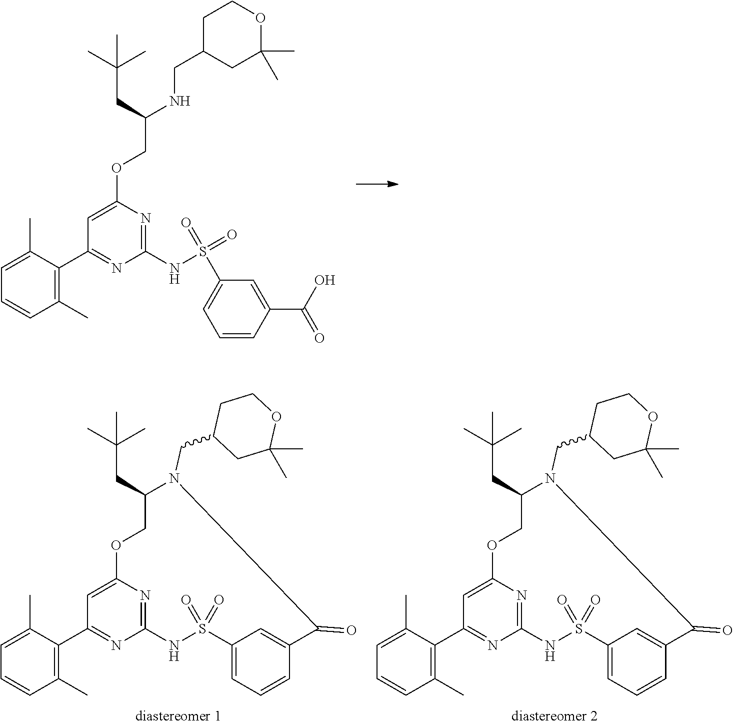

3-(N-(4-(2,6-dimethylphenyl)-6-(((2R)-2-(((2,2-dimethyltetrahydro-2H-pyran-4-yl)methyl)amino)-4,4-dimethylpentyl)oxy)pyrimidin-2-yl)sulfamoyl)benzoic acid (84 mg, 0.1315 mmol) and 2-chloro-4,6-dimethoxy-1,3,5-triazine (35 mg, 0.1993 mmol) were combined and dissolved in DMF (2.5 mL). The solution was cooled to 0° C. before the addition of 4-methylmorpholine (67 mg, 0.6624 mmol). The reaction mixture was stirred overnight at room temperature. After filtration, the product was isolated by UV-triggered reverse-phase HPLC eluting with a 30-60% acetonitrile/water gradient over 30 minutes with 5 mM acid modifier in the aqueous phase. After drying, the material was further purified on UV-triggered reverse-phase HPLC eluting with a 30-45% acetonitrile/water gradient over 30 minutes with 5 mM acid modifier in the aqueous phase, to give two isomers: (11R)-12-[(2,2-dimethyloxan-4-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (1.3 mg, 3%) diastereomer 1 was obtained as a white solid. ESI-MS m/z calc. 620.3032, found 621.4 (M+1)⁺; Retention time: 1.84 minutes; LC method A, and (11R)-12-[(2,2-dimethyloxan-4-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (5.1 mg, 12%) diastereomer 2 was obtained as a white solid. ESI-MS m/z calc. 620.3032, found 621.3 (M+1)⁺; Retention time: 1.87 minutes; LC method A.

### Example 20: Preparation of Compound 30

### Step 1: 3-[[4-(2,6-Dimethylphenyl)-6-[(2R)-4,4-dimethyl-2-[(2,2,6,6-tetramethyltetrahydropyran-4-yl)methylamino]pentoxy]pyrimidin-2-yl]sulfamoyl]benzoic acid

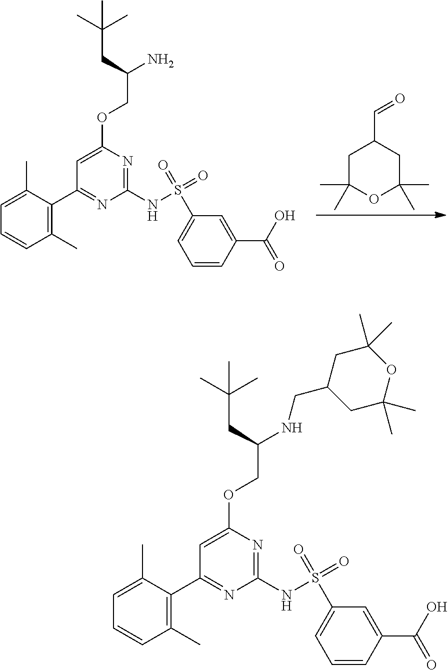

3-[[4-[(2R)-2-amino-4,4-dimethyl-pentoxy]-6-(2,6-dimethylphenyl)pyrimidin-2-yl]sulfamoyl]benzoic acid (hydrochloride salt) (50 mg, 0.08651 mmol) and 2,2,6,6-tetramethyltetrahydropyran-4-carbaldehyde (18 mg, 0.1057 mmol) were combined and suspended in dichloromethane (0.5 mL). The mixture was stirred at room temperature for 30 minutes. Sodium triacetoxyborohydride (Sodium salt) (37 mg, 0.1746 mmol) was added. and the reaction mixture was stirred at room temperature for 30 minutes before additional sodium triacetoxyborohydride (Sodium salt) (74 mg, 0.3492 mmol) was added. The final reaction mixture was stirred for 1 hour. Minimal aqueous 1 M HCl was added to quench the reaction. The product was isolated by UV-triggered reverse-phase HPLC eluting with a 10-99% acetonitrile/water gradient over 15 minutes with 5 mM acid modifier in the aqueous phase. 3-[[4-(2,6-dimethylphenyl)-6-[(2R)-4,4-dimethyl-2-[(2,2,6,6-tetramethyltetrahydropyran-4-yl)methylamino]pentoxy]pyrimidin-2-yl]sulfamoyl]benzoic acid (46 mg, 80%) was obtained as a white solid. ESI-MS m/z calc. 666.3451, found 667.4 (M+1)⁺; Retention time: 1.36 minutes; LC method A.

### Step 2: (11R)-6-(2,6-Dimethylphenyl)-11-(2,2-dimethylpropyl)-12-[(2,2,6,6-tetramethyloxan-4-yl)methyl]-9-oxa-2λ⁶-thia-3,5,12,19-tetraazatricyclo[12.3.1.14,8]nonadeca-1(18),4,6,8(19),14,16-hexaene-2,2,13-trione (Compound 30)

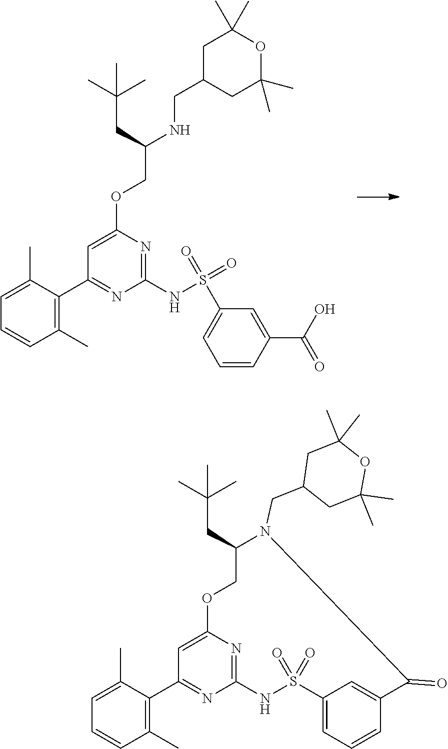

(R)-3-(N-(4-((4,4-dimethyl-2-(((2,2,6,6-tetramethyltetrahydro-2H-pyran-4-yl)methyl)amino)pentyl)oxy)-6-(2,6-dimethylphenyl)pyrimidin-2-yl)sulfamoyl)benzoic acid (46 mg, 0.06898 mmol) and 2-chloro-4,6-dimethoxy-1,3,5-triazine (18 mg, 0.1025 mmol) were combined and dissolved in DMF (1.4 mL). The solution was cooled to 0° C. before the addition of 4-methylmorpholine (35 mg, 0.3460 mmol). The reaction mixture was allowed to stir overnight at room temperature. After filtration, the product was isolated by UV-triggered reverse-phase HPLC eluting with a 30-60% acetonitrile/water gradient over 30 minutes with 5 mM acid modifier in the aqueous phase. (11R)-6-(2,6-Dimethylphenyl)-11-(2,2-dimethylpropyl)-12-[(2,2,6,6-tetramethyloxan-4-yl)methyl]-9-oxa-2λ⁶-thia-3,5,12,19-tetraazatricyclo[12.3.1.14,8]nonadeca-1(18),4,6,8(19),14,16-hexaene-2,2,13-trione (2.7 mg, 6%) was obtained as a white solid. ESI-MS m/z calc. 648.33453, found 649.4 (M+1)⁺; Retention time: 2.05 minutes, LC method A.

### Example 21: Preparation of Compound 31 and Compound 32

### Step 1: (6,6-Dimethyltetrahydropyran-3-yl)methanol

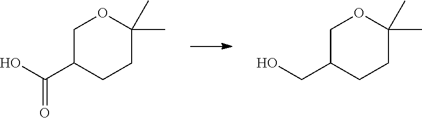

To a suspension of lithium aluminum hydride (910 mg, 22.777 mmol) in THE (25 mL) cooled at 0° C. was added a solution of 6,6-dimethyltetrahydropyran-3-carboxylic acid (3 g, 18.964 mmol) in THE (12 mL) over 10 minutes. The reaction mixture was stirred at 0° C. for 1 hour then at room temperature for 2 hours. The reaction mixture was then cooled down to 0° C., diluted with ether (50 mL) and water (4 mL) was added dropwise. An aqueous solution of NaOH (2 N, 4 mL) was subsequently added followed by water (12 mL). The reaction mixture was then stirred at room temperature and sodium sulfate (2 g) was added. After 2 hours, the mixture was filtered, rinsing the cake with ether (2×50 mL). The filtrate was concentrated in vacuo. Afforded (6,6-dimethyltetrahydropyran-3-yl)methanol (4.2 g, 100%) as a colorless oil that contains 35 mol % of THF by ¹H NMR. ¹H NMR (400 MHz, CDCl₃) δ 3.84-3.76 (m, 1H), 3.55-3.48 (m, 2H), 3.48-3.40 (m, 1H), 2.12-1.93 (m, 1H), 1.78-1.63 (m, 2H), 1.57-1.35 (m, 3H), 1.22 (s, 3H), 1.18 (s, 3H). (GC method 1B): Retention time: 5.73 minutes. ESI-MS m z calc. 144.11504, found 145.4 (M+1)⁺; Retention time: 1.29 minutes; LC method X.

### Step 2: 6,6-Dimethyltetrahydropyran-3-carbaldehyde

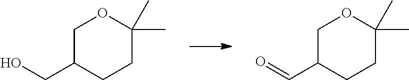

To a 0° C. solution of (6,6-dimethyltetrahydropyran-3-yl)methanol (250 mg, 1.1268 mmol) in water saturated DCM (10 mL) was added Dess-Martin periodinane (955 mg, 2.2516 mmol) and the reaction was then stirred for 2 hours at room temperature. A mixture of aqueous saturated solutions of sodium thiosulfate (5 mL), saturated sodium bicarbonate (5 mL), water (2 mL) and 1N NaOH (4 mL, to reach pH=9) was added and the reaction mixture was stirred for 10 minutes. The phases were separated and the aqueous one was extracted with DCM (3×10 mL). The combined organic layers were dried over sodium sulfate, filtered and concentrated in vacuo. Afforded 6,6-dimethyltetrahydropyran-3-carbaldehyde (235.6 mg, 96%) as a colorless oil. ¹H NMR (400 MHz, CHLOROFORM-d) δ 9.75 (s, 1H), 4.00-3.86 (m, 2H), 2.47-2.33 (m, 1H), 1.96-1.88 (m, 2H), 1.62-1.53 (m, 1H), 1.51-1.43 (m, 1H), 1.24 (s, 3H), 1.18 (s, 3H).

### Step 3: 3-[[4-(2,6-Dimethylphenyl)-6-[(2R)-2-[(6,6-dimethyltetrahydropyran-3-yl)methylamino]-4,4-dimethyl-pentoxy]pyrimidin-2-yl]sulfamoyl]benzoic acid

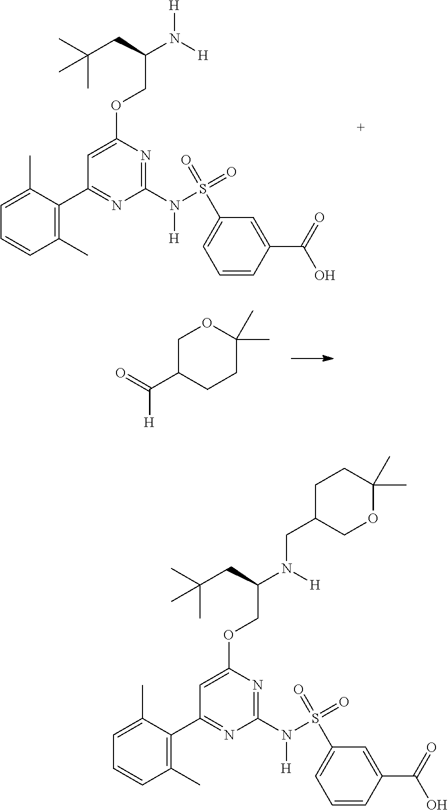

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (200 mg, 0.3642 mmol) in dichloromethane (2.5 mL) was added a solution of 6,6-dimethyltetrahydropyran-3-carbaldehyde (120 mg, 0.5485 mmol) in dichloromethane (2.5 mL) and the reaction was stirred at room temperature for 30 minutes. Sodium triacetoxyborohydride (390 mg, 1.8401 mmol) was added and the reaction was stirred at room temperature for 1.5 hours. 1 N aqueous HCl was added (10 mL) and the phases were separated. The aqueous layer was extracted with EtOAc (3×10 mL). The combined organic layers were dried over sodium sulfate, filtered and concentrated in vacuo. Afforded 3-[[4-(2,6-dimethylphenyl)-6-[(2R)-2-[(6,6-dimethyltetrahydropyran-3-yl)methylamino]-4,4-dimethyl-pentoxy]pyrimidin-2-yl]sulfamoyl]benzoic acid (hydrochloride salt) (365.9 mg, 100%) as a yellow oil. ESI-MS m/z calc. 638.3138, found 639.3 (M+1)⁺; Retention time: 2.65 minutes; LC method Y.

### Step 4: (11R)-6-(2,6-Dimethylphenyl)-11-(2,2-dimethylpropyl)-12-[(6,6-dimethyltetrahydropyran-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 31), and (11R)-6-(2,6-dimethylphenyl)-11-(2,2-dimethylpropyl)-12-[(6,6-dimethyltetrahydropyran-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 32)

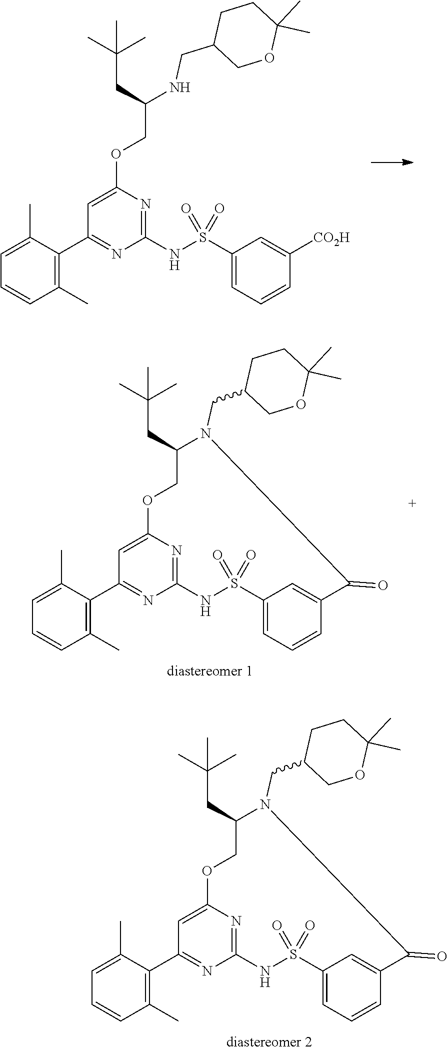 [SMILES: Cc1cccc(C)c1-c1cc(OC[C@@H](CC(C)(C)C)NCC2CCC(C)(C)OC2)nc(NS(=O)(=O)c2cccc(C(=O)O)c2)n1 ; Cc1cccc(C)c1-c1cc2nc(n1)NS(=O)(=O)c1cccc(c1)C(=O)N(CC1CCC(C)(C)OC1)[C@H](CC(C)(C)C)CO2 ; Cc1cccc(C)c1-c1cc2nc(n1)NS(=O)(=O)c1cccc(c1)C(=O)N(CC1CCC(C)(C)OC1)[C@H](CC(C)(C)C)CO2]

To a 0° C. solution of N-methylmorpholine (55.200 mg, 60 μL, 0.5457 mmol) in DMF (11 mL) was added 2-chloro-4,6-dimethoxy-1,3,5-triazine (320 mg, 1.8226 mmol) followed by 3-[[4-(2,6-dimethylphenyl)-6-[(2R)-2-[(6,6-dimethyltetrahydropyran-3-yl)methylamino]-4,4-dimethyl-pentoxy]pyrimidin-2-yl]sulfamoyl]benzoic acid (hydrochloride salt) (365 mg, 0.3621 mmol). After 5 minutes the reaction was warmed to room temperature and was stirred at this temperature for 48 hours. To this mixture was added 2-chloro-4,6-dimethoxy-1,3,5-triazine (320 mg, 1.8226 mmol) and N-methylmorpholine (55.200 mg, 60 μL, 0.5457 mmol) and the solution was stirred 48 hours at room temperature. The reaction was then concentrated under reduced pressure at 50° C. The remaining crude was diluted with DCM (50 mL) and the solution was washed with a 1:1 v/v mix of water and brine (4×40 mL), water (50 mL) and brine (50 mL). The resulting organic layer was dried over sodium sulfate, filtered and concentrated in vacuo. The crude was purified twice by reverse phase chromatography using a 50 g cartridge, eluting with a gradient of MeCN in water (containing 0.1% of formic acid) (5% for 5 CV then 50 to 100% in 20 CV) and by normal phase chromatography using a 12 g cartridge, eluting with a gradient of EtOAc in DCM (0 to 50%). The mixture was then purified by reverse phase chromatography using a 50 g cartridge, eluting with a gradient of MeCN in water (containing 0.1% of formic acid) (5% for 5 CV then 50 to 95% in 20 CV). The both product was extracted with EtOAc (3×50 mL), dried over anhydrous sodium sulfate, filtered and concentrated in vacuo. Afforded after lyophilization (11R)-6-(2,6-dimethylphenyl)-11-(2,2-dimethylpropyl)-12-[(6,6-dimethyltetrahydropyran-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (12.1 mg, 5%) diastereomer 1 as a white solid, ESI-MS m/z calc. 620.3032, found 621.3 (M+1)⁺; Retention time: 4.5 minutes; LC method Y, ¹H NMR (400 MHz, DMSO-d₆) δ 8.47 (s, 1H), 7.87 (br s, 1H), 7.56 (br s, 2H), 7.15 (br s, 1H), 7.08-7.01 (m, 2H), 5.12-5.05 (m, 1H), 4.05 (br s, 2H), 3.58-3.51 (m, 1H), 3.47-3.41 (m, 1H), 3.39-3.35 (m, 1H), 2.89-2.79 (m, 1H), 2.06-1.91 (m, 6H), 1.78-1.52 (m, 4H), 1.52-1.30 (m, 4H), 1.18 (s, 3H), 1.12 (s, 3H), 0.49 (s, 9H); and (11R)-6-(2,6-dimethylphenyl)-11-(2,2-dimethylpropyl)-12-[(6,6-dimethyltetrahydropyran-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (14.9 mg, 7%) diastereomer 2 as a white solid, ESI-MS m/z calc. 620.3032, found 621.3 (M+1)⁺; Retention time: 4.61 minutes; LC method Y. ¹H NMR (400 MHz, DMSO-d₆) δ 8.45 (s, 1H), 7.87 (br s, 1H), 7.60 (br s, 2H), 7.18 (br s, 1H), 7.06 (br s, 2H), 5.12-4.99 (m, 1H), 4.20-3.88 (m, 2H), 3.67-3.61 (m, 1H), 3.51-3.42 (m, 2H), 3.05-2.93 (m, 1H), 2.10-1.92 (m, 6H), 1.76-1.53 (m, 4H), 1.49-1.29 (m, 4H), 1.18 (s, 3H), 1.14 (s, 3H), 0.49 (s, 9H).

### Example 22: Preparation of Compound 33 and Compound 34

### Step 1: 2-Methylhex-5-en-2-ol

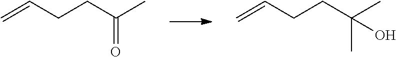

Methyl magnesium bromide (Solution in diethyl ether)) (115 mL of 3 M, 345.00 mmol) was diluted with diethyl ether (100 mL). hex-5-en-2-one (17.976 g, 21 mL, 183.16 mmol) was cautiously added dropwise at rt and the resulting mixture was stirred for 1 h at rt. An aqueous NH₄Cl (saturated solution, 50 mL) followed by aqueous NaHSO₄ (1.0 M, 50 mL). The two layers were separated and the aqueous one was extracted with diethyl ether (3×80 mL). The combined organic layers were washed with brine (50 mL), dried over magnesium sulfate, filtered and the solvent was removed in vacuo to give 2-methylhex-5-en-2-ol (23.26 g, 89%) as a colorless oil. ¹H NMR (400 MHz, CDCl₃) δ 5.90-5.73 (m, 1H), 5.06-4.87 (m, 2H), 2.21-2.02 (m, 2H), 1.58-1.49 (m, 2H), 1.21-1.17 (m, 6H).

### Step 2: (5,5-dDmethyltetrahydrofuran-2-yl)methanol

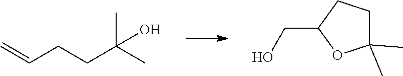

3-Chloroperoxybenzoic acid (25 g, 107.21 mmol) was added in portions to 2-methylhex-5-en-2-ol (10 g, 87.577 mmol) in dichloromethane (193 mL). The mixture was stirred at room temperature for 18 h. The precipitated acid was removed by filtration and the solution was concentrated under reduced pressure. Pentane (100 mL) was added to the residue then the precipitated acid was removed by filtration. The solution was concentrated and the crude product was purified on silica-gel (120 g) eluting with a gradient of EtOAc in heptane 10% for 3 CV then 20% 3 CV then 300% for 6 CV to yield (5,5-dimethyltetrahydrofuran-2-yl)methanol (9 g, 55%) as a yellow oil. ¹H NMR (400 MHz, CDCl₃) δ 4.18-4.10 (m, 1H), 3.70 (dd, J 11.5, 3.2 Hz, 1H), 3.51 (dd, J 11.5, 5.4 Hz, 1H), 2.04-1.94 (m, 1H), 1.88-1.74 (m, 3H), 1.29 (s, 3H), 1.27 (s, 3H).

### Step 3: 5,5-Dimethyltetrahydrofuran-2-carbaldehyde

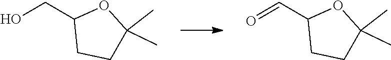

To a 0° C. solution of (5,5-dimethyltetrahydrofuran-2-yl)methanol (500 mg, 3.6487 mmol) in water saturated DCM (25 mL) was added Dess-Martin periodinane (1.6 g, 3.7723 mmol) and the reaction was stirred for 30 min at room temperature. A mixture of aqueous saturated solutions of sodium thiosulfate (10 mL), saturated sodium bicarbonate (10 mL), water (4 mL) and 1N NaOH (10 mL, to reach pH=9) were added and the reaction mixture was stirred for 5 min. The phases were separated and the aqueous one was extracted with DCM (3×10 mL). The combined organic layers were dried over sodium sulfate, filtered and concentrated under reduced pressure. DCM (25 mL) was added to the residue, then Dess-Martin periodinane (1.6 g, 3.7723 mmol) was added to the mixture at 0° C. The reaction mixture was stirred for 30 min at room temperature. A mixture of aqueous saturated solutions of sodium thiosulfate (10 mL), saturated sodium bicarbonate (10 mL), water (4 mL) and 1N NaOH (10 mL, to reach pH=9) were added and the reaction mixture was stirred for 5 min. The phases were separated and the aqueous one was extracted with DCM (3×10 mL). The combined organic layers were dried over sodium sulfate, filtered and concentrated under reduced pressure to afforded 5,5-dimethyltetrahydrofuran-2-carbaldehyde (450 mg, 60%) as a colorless oil. ¹H NMR (400 MHz, CDCl₃) δ 9.69 (d, J 1.7 Hz, 1H), 2.51-2.33 (m, 1H), 2.13-2.04 (m, 1H), 1.87-1.69 (m, 3H), 1.32 (s, 3H), 1.31 (s, 3H). GC-FID (GC method 1B): Retention time: 2.15 minutes.

### Step 4: 3-[[4-(2,6-Dimethylphenyl)-6-[(2R)-2-[(5,5-dimethyltetrahydrofuran-2-yl)methylamino]-4,4-dimethyl-pentoxy]pyrimidin-2-yl]sulfamoyl]benzoic acid

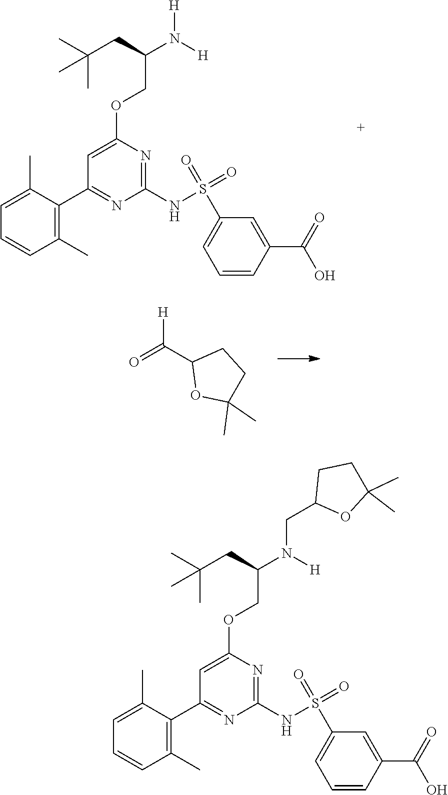

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (200 mg, 0.3642 mmol) in dichloromethane (3.5 mL) was added a solution of 5,5-dimethyltetrahydrofuran-2-carbaldehyde (75 mg, 0.4389 mmol) in dichloromethane (3.5 mL) and the reaction was stirred at room temperature for 45 min. Sodium triacetoxyborohydride (387 mg, 1.8260 mmol) was added and the reaction was stirred at room temperature for 1 h. 1 N aqueous HCl was added (15 mL) and the phases were separated. The aqueous layer was extracted with EtOAc (3×20 mL). The combined organic layers were dried over sodium sulfate, filtered and concentrated under reduced pressure. The residue was co-evaporated with heptanes (2×50 mL). Afforded 3-[[4-(2,6-dimethylphenyl)-6-[(2R)-2-[(5,5-dimethyltetrahydrofuran-2-yl)methylamino]-4,4-dimethyl-pentoxy]pyrimidin-2-yl]sulfamoyl]benzoic acid (hydrochloride salt) (235 mg, 32%) as a yellow semi-solid ESI-MS m/z calc. 624.2982, found 625.4 (M+1)⁺; Retention time: 1.53 minutes; LC method X.

### Step 5: (11R)-6-(2,6-Dimethylphenyl)-11-(2,2-dimethylpropyl)-12-[(5,5-dimethyltetrahydrofuran-2-yl)methyl]-2,2-dioxo-9-oxa-2⁶-thia-3,5,12,19-tetrazatricyclo[12.3.1.14,8]nonadeca-1(18),4(19),5,7,14,16-hexaen-13-one

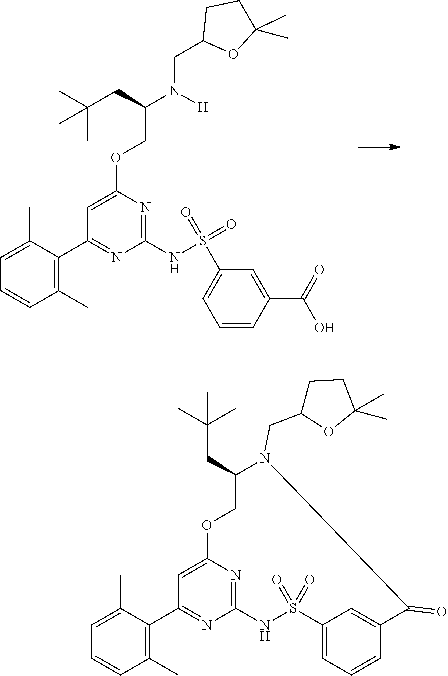 [SMILES: Cc1cccc(C)c1-c1cc(OC[C@@H](CC(C)(C)C)NCC2CCC(C)(C)O2)nc(NS(=O)(=O)c2cccc(C(=O)O)c2)n1 ; Cc1cccc(C)c1-c1cc2nc(n1)NS(=O)(=O)c1cccc(c1)C(=O)N(CC1CCC(C)(C)O1)[C@H](CC(C)(C)C)CO2]

To a 0° C. solution of N-methylmorpholine (184.00 mg, 0.2 mL, 1.8191 mmol) in DMF (31 mL) was added 2-chloro-4,6-dimethoxy-1,3,5-triazine (97 mg, 0.5525 mmol) followed by 3-[[4-(2,6-dimethylphenyl)-6-[(2R)-2-[(5,5-dimethyltetrahydrofuran-2-yl)methylamino]-4,4-dimethyl-pentoxy]pyrimidin-2-yl]sulfamoyl]benzoic acid (hydrochloride salt) (235 mg, 0.3554 mmol). After 5 min the reaction was stirred at room temperature for 72 h. The reaction mixture was concentrated under reduced pressure at 50° C. The remaining crude was diluted with DCM (50 mL) and the solution was washed with a 1:1 v/v mix of water and brine (4×20 mL), water (25 mL) and brine (50 mL). The resulting organic layer was dried over sodium sulfate, filtered and concentrated under reduced pressure. The crude was purified by flash chromatography using a 24 g cartridge, eluting with a gradient of EtOAc in DCM (5 to 50% in 25 CV). Removal of the volatiles under reduced pressure afforded a white solid which was purified by reverse phase chromatography on a 15.5 g C₁₈ cartridge using a gradient of MeCN in acidic water (0.1% v/v of formic acid in water) of 40 to 100% for 15 CV then 100% for 5 CV. The fractions containing the product were evaporated and then lyophilized. Afforded (11R)-6-(2,6-dimethylphenyl)-11-(2,2-dimethylpropyl)-12-[(5,5-dimethyltetrahydrofuran-2-yl)methyl]-2,2-dioxo-9-oxa-2?⁶-thia-3,5,12,19-tetrazatricyclo[12.3.1.14,8]nonadeca-1(18),4(19),5,7,14,16-hexaen-13-one (28.6 mg, 13%) as a white solid. ESI-MS m/z calc. 606.2876, found 607.3 (M+1)⁺; Retention time: 4.72 minutes; LC method Y; ¹H NMR (400 MHz, DMSO-d₆) δ 13.23-12.88 (m, 1H), 8.46 (d, J 19.8 Hz, 1H), 8.02-7.89 (m, 1H), 7.76-7.59 (m, 2H), 7.33-7.21 (m, 1H), 7.17-7.04 (m, 2H), 6.49-6.28 (m, 1H), 5.18-5.01 (m, 1H), 4.38-4.15 (m, 2H), 4.00-3.86 (m, 1H), 3.82-3.71 (m, 1H), 3.61 (br dd, J 14.1, 5.5 Hz, 1H), 3.36 (br dd, J 14.2, 4.4 Hz, 1H), 3.07-2.93 (m, 1H), 2.20-1.88 (m, 6H), 1.81-1.62 (m, 3H), 1.43-1.00 (m, 7H), 0.51 (s, 9H).

### Step 6: (11R)-12-[(5,5-Dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 33), and (11R)-12-[(5,5-dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 2 (Compound 34)

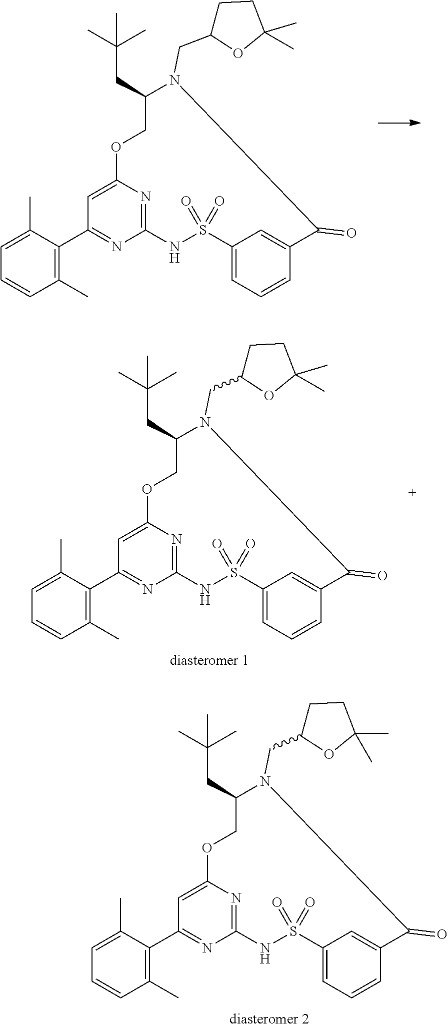 [SMILES: Cc1cccc(C)c1-c1cc2nc(n1)NS(=O)(=O)c1cccc(c1)C(=O)N(CC1CCC(C)(C)O1)[C@H](CC(C)(C)C)CO2 ; Cc1cccc(C)c1-c1cc2nc(n1)NS(=O)(=O)c1cccc(c1)C(=O)N(CC1CCC(C)(C)O1)[C@H](CC(C)(C)C)CO2 ; Cc1cccc(C)c1-c1cc2nc(n1)NS(=O)(=O)c1cccc(c1)C(=O)N(CC1CCC(C)(C)O1)[C@H](CC(C)(C)C)CO2]

(11R)-12-[(5,5-dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (23.6 mg, 0.03889 mmol) (60:40 isomer mixture) was dissolved in DMSO (1 mL). Purification by reverse phase HPLC (1-99% acetonitrile/5 mM HCl over 45 min, providing two enriched fractions that were purified a second time) provided two separated diastereomers as a white solid: First to elute, more polar isomer: (11R)-12-[(5,5-dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (9.1 mg, 64%) diastereomer 1. ESI-MS m z calc. 606.2876, found 607.39 (M+1)⁺; Retention time: 2.07 minutes; LC method A, ¹H NMR (400 MHz, DMSO-d₆) δ 13.47-11.41 (broad m, 1H), 8.49 (s, 1H), 7.93 (s, 1H), 7.66 (s, 2H), 7.39-7.19 (m, 1H), 7.12 (s, 2H), 6.39 (s, 1H), 5.09 (dd, J=11.0, 4.3 Hz, 1H), 4.35-4.13 (m, 2H), 3.77 (t, J 11.4 Hz, 1H), 3.61 (dd, J 14.1, 5.5 Hz, 1H), 3.40-3.34 (m, 1H), 2.24-1.84 (m, 8H), 1.83-1.61 (m, 3H), 1.29 (d, J=15.1 Hz, 1H), 1.25 (s, 3H), 1.19 (s, 3H), 0.51 (s, 9H), and a second to elute, less polar isomer: (11R)-12-[(5,5-dimethyloxolan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (6.6 mg, 70%) diastereomer 2. ESI-MS m/z calc. 606.2876, found 607.65 (M+1)⁺; Retention time: 2.08 minutes; LC method A. ¹H NMR (400 MHz, DMSO-d₆) δ 13.63-11.43 (broad m, 1H), 8.44 (s, 1H), 7.94 (s, 1H), 7.68 (s, 2H), 7.33-7.21 (m, 1H), 7.19-6.97 (m, 2H), 6.40 (s, 1H), 5.08 (dd, J 11.5, 4.2 Hz, 1H), 4.37-4.17 (m, 2H), 4.01-3.88 (m, 1H), 3.76 (dd, J=13.9, 2.3 Hz, 1H), 2.98 (dd, J=13.9, 9.2 Hz, 1H), 2.25-1.86 (m, 7H), 1.83-1.59 (m, 4H), 1.37-1.31 (m, 1H), 1.30 (s, 3H), 1.19 (s, 3H), 0.51 (s, 9H).

### Example 23: Preparation of Compound 35 and Compound 36

### Step 1: 3,3-Dimethyltetrahydropyran-2-one

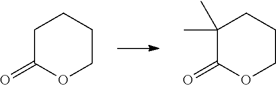

LiHMDS (1M in THF) (112 mL of 1 M, 112.00 mmol) was added to a solution of tetrahydropyran-2-one (5 g, 49.942 mmol) and MeI (29.640 g, 13 mL, 208.82 mmol) in THE (150 mL) at −78° C. The reaction was then allowed to slowly warm to room temperature. After stirring overnight, the reaction mixture was quenched with saturated aq. NH₄Cl (100 mL). After 15 min, the layers were separated, and the aqueous layer was extracted with EtOAc (3×100 mL). The organic layer and extracts were combined, dried over anhydrous sodium sulfate, filtered, and concentrated under reduced pressure. The residue was purified on a silica gel column using 0 then 20% ethyl acetate in heptane to provide 3,3-dimethyltetrahydropyran-2-one (3.77 g, 56%) as a clear pale-yellow oil. ¹H NMR (400 MHz, CHLOROFORM-d) δ 4.40-4.28 (m, 2H), 1.97-1.86 (m, 2H), 1.78-1.72 (m, 2H), 1.35-1.27 (m, 6H). ESI-MS m/z calc. 128.08372, found 129.4 (M+1)⁺; Retention time: 1.35 minutes; LC method X.

### Step 2: 3,3-Dimethyl-2-(p-tolylsulfinylmethyl)tetrahydropyran-2-ol

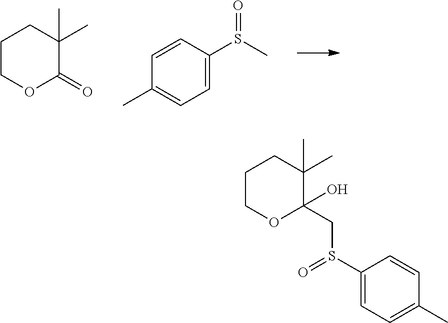

LDA solution in hexane/THF/ethylbenzene (25.3 mL of 2 M, 50.600 mmol) was added dropwise to a solution of 1-methyl-4-methylsulfinyl-benzene (3.91 g, 25.352 mmol) in THE (78 mL) with stirring at −78° C. under inert atmosphere. After 30 min, a solution of 3,3-dimethyltetrahydropyran-2-one (3.25 g, 24.089 mmol) in THE (16 mL) was added to the mixture at −78° C. and the stirring was continued at same temperature for 1 h. The reaction was quenched with saturated ammonium chloride aqueous solution (100 mL) and extracted with Ethyl acetate (3×100 mL). The extract was washed with water and brine (2×50 mL), dried over sodium sulfate, filtered and concentrated to dryness. The crude was purified by flash chromatography (120 g column) eluting with 0 to 40% ethyl acetate in heptanes to afford 3,3-dimethyl-2-(p-tolylsulfinylmethyl)tetrahydropyran-2-ol (5.78 g, 85%) as a white solid. ¹H NMR (400 MHz, CDCl₃) δ 7.59 (d, J 8.3 Hz, 2H), 7.36 (d, J 7.8 Hz, 2H), 5.67 (s, 1H), 4.18 (ddd, J 12.8, 11.2, 2.9 Hz, 1H), 3.80 (dd, J 11.1, 5.3 Hz, 1H), 2.98 (d, J 12.7 Hz, 1H), 2.81 (d, J 12.7 Hz, 1H), 2.44 (s, 3H), 2.06-1.96 (m, 1H), 1.96-1.82 (m, 1H), 1.46-1.39 (m, 1H), 1.27-1.17 (m, 1H), 1.03 (s, 3H), 0.93 (s, 3H). ESI-MS m/z calc. 282.129, found 265.1 (M-17)⁺; Retention time: 2.49 minutes, LC method 1D.

### Step 3: 3,3-Dimethyl-2-(p-tolylsulfinylmethyl)tetrahydropyran

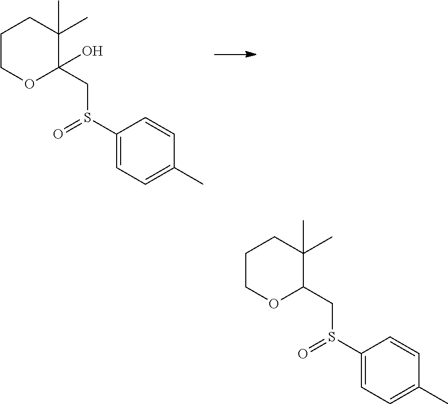 [SMILES: Cc1ccc(S(=O)CC2(O)OCCCC2(C)C)cc1 ; Cc1ccc(S(=O)CC2OCCCC2(C)C)cc1]

To a solution of 3,3-dimethyl-2-(p-tolylsulfinylmethyl)tetrahydropyran-2-ol (5.78 g, 20.468 mmol) in dichloromethane (75 mL) was added boron trifluoride ethyl etherate (8.9700 g, 7.8 mL, 63.201 mmol) followed by triethylsilane (5.0232 g, 6.9 mL, 43.200 mmol). The mixture was stirred at room temperature for 18 hours. The mixture was quenched with a saturated aqueous solution of ammonium chloride (100 mL). The phases were separated, and the aqueous layer was extracted with ethyl acetate (2×100 mL). The organic phase was dried over sodium sulfate, filtered and concentrated to dryness. The crude oil was purified by flash chromatography on silica gel (100 g column) eluting with 0% to 50% to afford a 2:1 diastereomeric mixture of 3,3-dimethyl-2-(p-tolylsulfinylmethyl)tetrahydropyran (4.67 g, 84%) as a yellowish solid. ESI-MS m/z calc. 266.1341, found 267.1 (M+1)⁺; Retention time: 3.9 minutes (major). ESI-MS m/z calc. 266.1341, found 267.2 (M+1)⁺; Retention time 3.82 minutes (minor), LC method Y.

### Step 4: 3,3-Dimethyltetrahydropyran-2-carbaldehyde

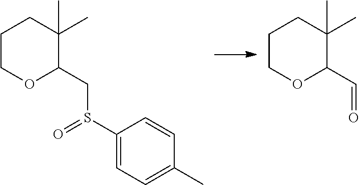

To a solution of 3,3-dimethyl-2-(p-tolylsulfinylmethyl)tetrahydropyran (309 mg, 1.1588 mmol) and 2,6-lutidine (425.50 mg, 0.46 mL, 3.9710 mmol) in acetonitrile (13.5 mL) was added trifluoroacetic anhydride (710.17 mg, 0.47 mL, 3.3813 mmol) at 0° C. After stirring at this temperature for 15 minutes, copper (II) chloride (315 mg, 2.3428 mmol) in water (2.8 mL) was added and the mixture was stirred for 16 hours. The mixture was partitioned between dichloromethane (40 mL) and water (25 mL). The organic layer was washed with brine (2×25 mL), dried over sodium sulfate, filtered and concentrated to afford crude 3,3-dimethyltetrahydropyran-2-carbaldehyde (390 mg, 95%) as a yellow oil. ¹H NMR (400 MHz, CDCl₃) δ 9.63 (d, J 1.5 Hz, 1H), 4.16-4.10 (m, 1H), 3.50 (d, J 1.0 Hz, 1H), 3.46-3.38 (m, 1H), 1.55-1.41 (m, 4H), 1.09 (s, 3H), 1.02 (s, 3H).

### Step 5: 3-[[4-(2,6-Dimethylphenyl)-6-[(2R)-2-[(3,3-dimethyltetrahydropyran-2-yl)methylamino]-4,4-dimethyl-pentoxy]pyrimidin-2-yl]sulfamoyl]benzoic acid

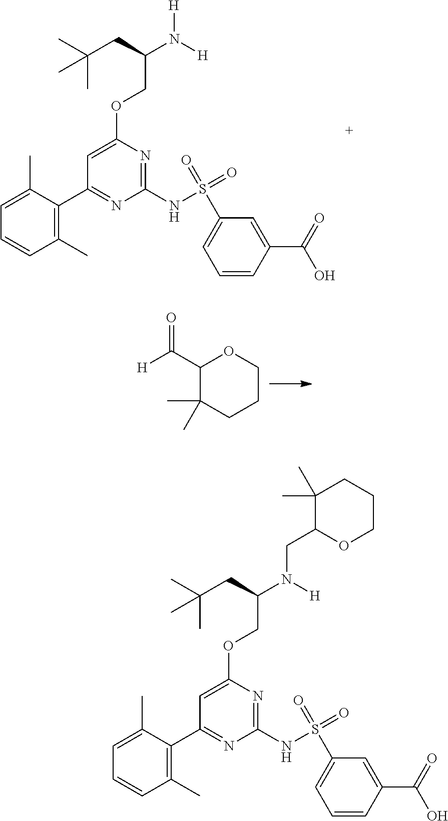

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (100 mg, 0.1821 mmol) in dichloromethane (1.25 mL) was added a solution of 3,3-dimethyltetrahydropyran-2-carbaldehyde (146 mg, 0.3696 mmol) in dichloromethane (1.25 mL) and the reaction was stirred at room temperature for 30 minutes. Sodium triacetoxyborohydride (195 mg, 0.9201 mmol) was added and the reaction was stirred at room temperature for 1 hour. 3,3-Dimethyltetrahydropyran-2-carbaldehyde (200 mg, 0.5063 mmol) and sodium triacetoxyborohydride (150 mg, 0.7077 mmol) were added and the mixture was stirred for 2 more hours. The mixture was quenched with 1 N aqueous HCl (10 mL) and the phases were separated. The aqueous layer was extracted with EtOAc (3×10 mL). The combined organic layers were dried over sodium sulfate, filtered and concentrated in vacuo to give crude 3-[[4-(2,6-dimethylphenyl)-6-[(2R)-2-[(3,3-dimethyltetrahydropyran-2-yl)methylamino]-4,4-dimethyl-pentoxy]pyrimidin-2-yl]sulfamoyl]benzoic acid (hydrochloride salt) (298 mg, 242%) as a yellow oil. ESI-MS m/z calc. 638.3138, found 639.4 (M+1)⁺; Retention time: 1.57 minutes; LC method X.

### Step 6: (11R)-6-(2,6-Dimethylphenyl)-11-(2,2-dimethylpropyl)-12-[(3,3-dimethyltetrahydropyran-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

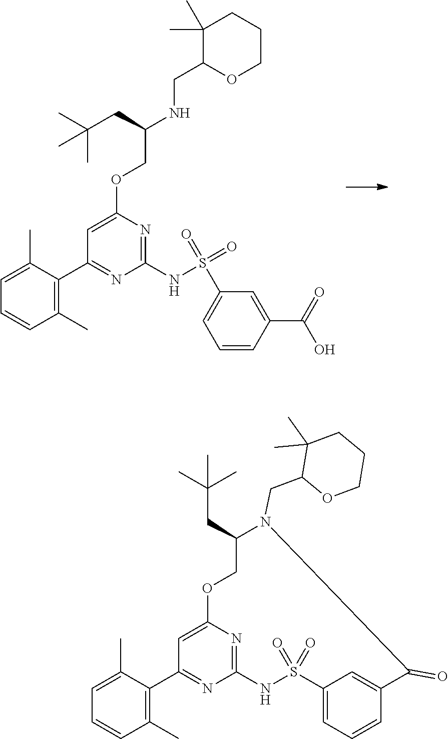 [SMILES: Cc1cccc(C)c1-c1cc(OC[C@@H](CC(C)(C)C)NCC2OCCCC2(C)C)nc(NS(=O)(=O)c2cccc(C(=O)O)c2)n1 ; Cc1cccc(C)c1-c1cc2nc(n1)NS(=O)(=O)c1cccc(c1)C(=O)N(CC1OCCCC1(C)C)[C@H](CC(C)(C)C)CO2]

To a stirring solution of N-methylmorpholine (193.20 mg, 0.21 mL, 1.9101 mmol) in DMF (22 mL) at 0° C. was added 2-chloro-4,6-dimethoxy-1,3,5-triazine (117 mg, 0.6664 mmol) followed by 3-[[4-(2,6-dimethylphenyl)-6-[(2R)-2-[(3,3-dimethyltetrahydropyran-2-yl)methylamino]-4,4-dimethyl-pentoxy]pyrimidin-2-yl]sulfamoyl]benzoic acid (hydrochloride salt) (231 mg, 0.3168 mmol) in DMF (8 mL). After 5 min the reaction was stirred at room temperature for 18 h. The solvent was removed under reduced pressure at 50° C. The remaining crude was diluted with dichloromethane (75 mL) and the solution was washed with a 1:1 v/v mixture of water and brine (3×40 mL). The organic layer was dried over sodium sulfate, filtered and concentrated under reduced pressure. The crude was purified by flash chromatography (40 g column), eluting with a gradient of ethyl acetate in dichloromethane (0 to 50%). The remaining oil was purified by reverse phase chromatography (Cis 50 g column) using a gradient of acetonitrile in acidic water (0.1% v/v of formic acid in water). The fractions containing the product were evaporated and then lyophilized to give a 3:2 diastereoisomeric mixture of (11R)-6-(2,6-dimethylphenyl)-11-(2,2-dimethylpropyl)-12-[(3,3-dimethyltetrahydropyran-2-yl)methyl]-2,2-dioxo-9-oxa-2?⁶-thia-3,5,12,19-tetrazatricyclo[12.3.1.14,8]nonadeca-1(18),4(19),5,7,14,16-hexaen-13-one (94.5 mg, 48%) as a white solid. ESI-MS m/z calc. 620.3032, found 621.3 (M+1)⁺; Retention time: 5.0 minutes (LC method Y); ¹H NMR (400 MHz, DMSO-d₆) δ 12.29 (br s, 1H), 8.55-8.47 (m, 1H), 7.99-7.87 (m, 1H), 7.73-7.56 (m, 2H), 7.23 (t, J 7.3 Hz, 1H), 7.10 (d, J 7.6 Hz, 2H), 6.29 (s, 1H), 5.15-5.06 (m, 1H), 4.18-4.06 (m, 1H), 4.04-3.87 (m, 2H), 3.74-3.51 (m, 2H), 3.42-3.26 (m, 2H), 2.24 (dd, J 14.9, 8.3 Hz, 1H), 2.01 (s, 6H), 1.80-1.62 (m, 1H), 1.51-1.24 (m, 4H), 1.07 (s, 3H), 1.00 (s, 3H), 0.53 (s, 9H) (major isomer). 12.29 (br s, 1H), 8.55-8.47 (m, 1H), 7.99-7.87 (m, 1H), 7.73-7.56 (m, 2H), 7.23 (t, J 7.3 Hz, 1H), 7.10 (d, J 7.6 Hz, 2H), 6.29 (s, 1H), 5.15-5.06 (m, 1H), 4.18-4.06 (m, 1H), 4.04-3.87 (m, 2H), 3.74-3.51 (m, 2H), 3.42-3.26 (m, 2H), 2.84-2.74 (m, 1H), 2.01 (s, 6H), 1.80-1.62 (m, 1H), 1.51-1.24 (m, 4H), 1.02 (s, 3H), 1.01 (s, 3H), 0.55 (s, 9H) (minor isomer).

### Step 7: (11R)-12-[(3,3-Dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 35), and (11R)-12-[(3,3-dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 2 (Compound 36)

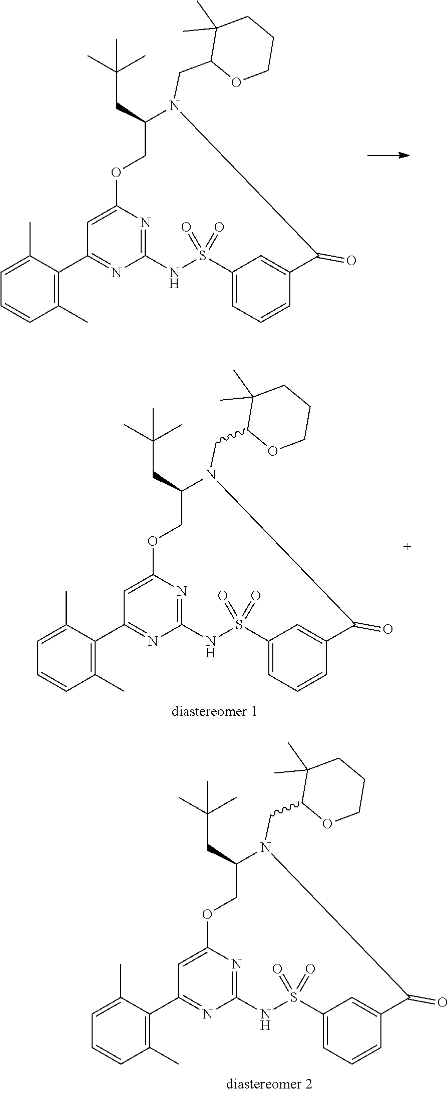 [SMILES: Cc1cccc(C)c1-c1cc2nc(n1)NS(=O)(=O)c1cccc(c1)C(=O)N(CC1OCCCC1(C)C)[C@H](CC(C)(C)C)CO2 ; Cc1cccc(C)c1-c1cc2nc(n1)NS(=O)(=O)c1cccc(c1)C(=O)N(CC1OCCCC1(C)C)[C@H](CC(C)(C)C)CO2 ; "+" ; Cc1cccc(C)c1-c1cc2nc(n1)NS(=O)(=O)c1cccc(c1)C(=O)N(CC1OCCCC1(C)C)[C@H](CC(C)(C)C)CO2]

(11R)-12-[(3,3-dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (89 mg, 0.1434 mmol) (60:40 mixture of diastereomers) was dissolved in DMSO (2 mL). Purification by reverse phase HPLC (10-99% acetonitrile/5 mM HCl over 30 min) provided two isomers as a white solid: First to elute, more polar isomer: (11R)-12-[(3,3-dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (40.1 mg, 74%), diastereomer 1). ESI-MS m z calc. 620.3032, found 621.51 (M+1)⁺; Retention time: 2.21 minutes. LC method A, ¹H NMR (400 MHz, DMSO-d₆) δ 13.25-11.58 (broad m, 1H), 8.49 (s, 1H), 7.88 (s, 1H), 7.75-7.43 (m, 2H), 7.26 (t, J 7.7 Hz, 1H), 7.12 (s, 2H), 6.37 (s, 1H), 5.09 (dd, J 10.9, 4.6 Hz, 1H), 4.13 (t, J=11.3 Hz, 1H), 3.90 (dd, J=11.0, 4.5 Hz, 1H), 3.74-3.51 (m, 3H), 3.30-3.22 (m, 1H), 3.18 (dd, J 14.4, 8.5 Hz, 1H), 2.30 (dd, J 14.8, 8.0 Hz, 1H), 2.21-1.80 (m, 6H), 1.78-1.63 (m, 1H), 1.47-1.32 (m, 3H), 1.27-1.18 (m, 1H), 1.06 (s, 3H), 0.96 (s, 3H), 0.48 (s, 9H), and a second to elute, less polar isomer: (11R)-12-[(3,3-dimethyloxan-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (27.1 mg, 76%) diastereomer 2. ESI-MS m/z calc. 620.3032, found 621.4 (M+1)⁺; Retention time: 2.27 minutes; LC method A. ¹H NMR (400 MHz, DMSO-d₆) δ 13.38-11.68 (broad m, 1H), 8.43 (s, 1H), 7.94 (s, 1H), 7.68 (s, 2H), 7.26 (t, J 7.6 Hz, 1H), 7.12 (d, J 7.6 Hz, 2H), 6.37 (s, 1H), 5.04 (dd, J 11.4, 4.1 Hz, 1H), 4.13 (t, J 11.4 Hz, 1H), 3.99 (dd, J 11.1, 4.6 Hz, 1H), 3.95-3.76 (m, 2H), 3.36 (overlapped with water d, J 8.9 Hz, 1H), 2.76 (dd, J 14.1, 9.0 Hz, 1H), 2.27-1.80 (m, 6H), 1.77-1.57 (m, 2H), 1.51-1.18 (m, 5H), 1.00 (s, 3H), 0.98 (s, 3H), 0.51 (s, 9H).

### Example 24: Preparation of Compound 37

### Step 1: 3-[[4-(2,6-Dimethylphenyl)-6-[(2R)-4-methyl-2-[2-[1-(trifluoromethyl)cyclopropyl]ethylamino]pentoxy]pyrimidin-2-yl]sulfamoyl]benzoic acid

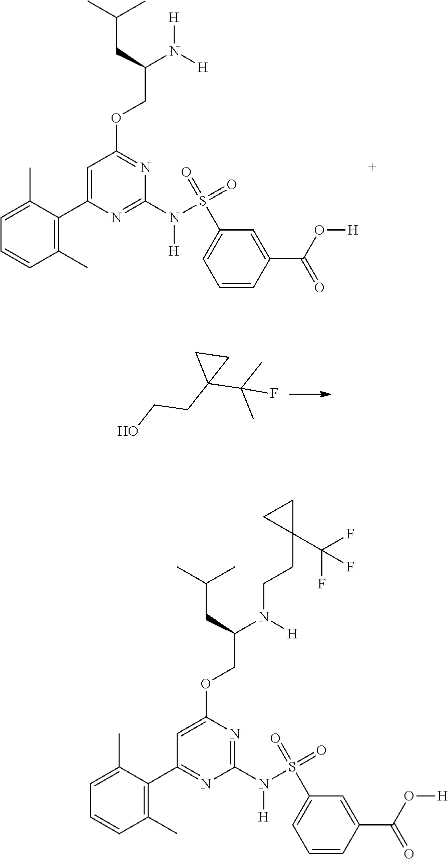

2-[1-(Trifluoromethyl)cyclopropyl]ethanol (approximately 38.66 mg, 0.2508 mmol) was combined with Dess-Martin periodinane (approximately 97.85 mg, 0.2307 mmol) in DCM (0.4 mL). This reaction mixture was stirred for 30 minutes at room temperature. A aliquot (0.2 mL) of the reaction mixture was then added by syringe to a second vial containing 3-[[4-[(2R)-2-amino-4-methyl-pentoxy]-6-(2,6-dimethylphenyl)pyrimidin-2-yl]sulfamoyl]benzoic acid (50 mg, 0.1003 mmol), and acetic acid (approximately 60.23 mg, 57.04 μL, 1.003 mmol) in DCE (0.3 mL). Sodium cyanoborohydride (approximately 50.42 mg, 0.8024 mmol) was added and the reaction mixture was stirred at room temperature for an additional 2 hours. The reaction mixture was then diluted with methanol, filtered, and purified by prep HPC (1-70% ACN in water, HCl modifier, 15 min run) to give the indicated 3-[[4-(2,6-dimethylphenyl)-6-[(2R)-4-methyl-2-[2-[1-(trifluoromethyl)cyclopropyl]ethylamino]pentoxy]pyrimidin-2-yl]sulfamoyl]benzoic acid (16 mg, 25%).ESI-MS m/z calc. 634.24365, found 635.4 (M+1)⁺; Retention time: 0.52 minutes; LC method D.

### Step 2: (11R)-6-(2,6-Dimethylphenyl)-11-(2-methylpropyl)-12-{2-[1-(trifluoromethyl)cyclopropyl]ethyl}-9-oxa-2λ⁶-thia-3,5,12,19-tetraazatricyclo[12.3.1.14,8]nonadeca-1(17),4(19),5,7,14(18),15-hexaene-2,2,13-trione (Compound 37)

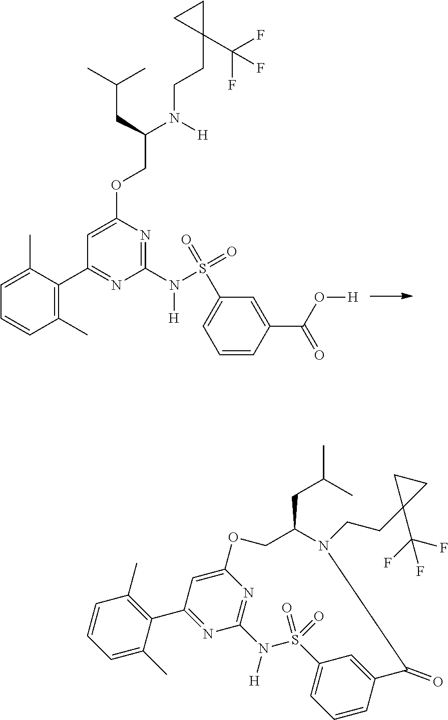 [SMILES: Cc1cccc(C)c1-c1cc(OC[C@@H](CC(C)C)NCCC2(C(F)(F)F)CC2)nc(NS(=O)(=O)c2cccc(C(=O)O)c2)n1 ; Cc1cccc(C)c1-c1cc2nc(n1)NS(=O)(=O)c1cccc(c1)C(=O)N(CCC1(C(F)(F)F)CC1)[C@H](CC(C)C)CO2]

3-[[4-(2,6-Dimethylphenyl)-6-[(2R)-4-methyl-2-[2-[1-(trifluoromethyl)cyclopropyl]ethylamino]pentoxy]pyrimidin-2-yl]sulfamoyl]benzoic acid (16 mg, 0.02521 mmol) was combined with HATU (approximately 11.50 mg, 0.03025 mmol) in anhydrous DMF (1 mL). DIPEA (approximately 16.28 mg, 21.94 μL, 0.1260 mmol) was added, and the reaction mixture was stirred at room temperature for one hour. The reaction mixture was then filtered and purified by reverse phase HPLC (1-99% ACN, HCl modifier, 15 min run) to give (11R)-6-(2,6-dimethylphenyl)-11-(2-methylpropyl)-12-{2-[1-(trifluoromethyl)cyclopropyl]ethyl}-9-oxa-2λ⁶-thia-3,5,12,19-tetraazatricyclo[12.3.1.14,8]nonadeca-1(17),4(19),5,7,14(18),15-hexaene-2,2,13-trione (5.7 mg, 37%) after drying. ESI-MS m/z calc. 616.2331, found 617.4 (M+1)⁺; Retention time: 2.05 minutes; LC method A.

### Example 25: Preparation of Compound 38

### Step 1: 3-[[4-[(2R)-2-[[(4R)-2,2-dimethyl-1,3-Dioxolan-4-yl]methylamino]-4-methyl-pentoxy]-6-(2,6-dimethylphenyl)pyrimidin-2-yl]sulfamoyl]benzoic acid

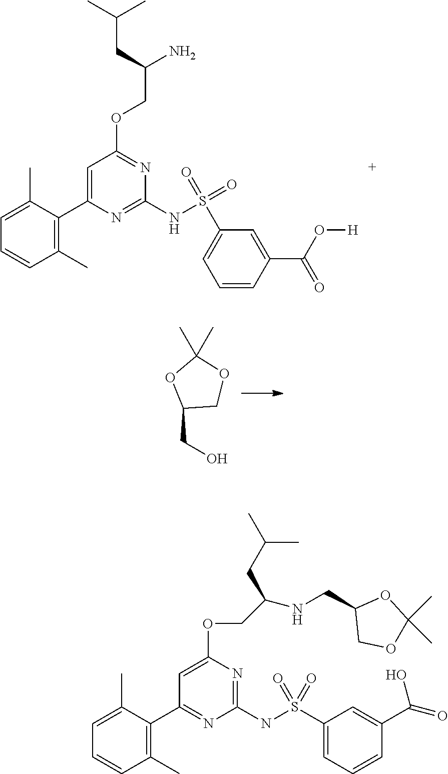

[(4R)-2,2-dimethyl-1,3-dioxolan-4-yl]methanol (approximately 66.28 mg, 0.5015 mmol) was combined with Dess-Martin periodinane (approximately 212.7 mg, 0.5015 mmol) in DCM (0.4 mL). This reaction mixture was stirred for 30 minutes at room temperature. 0.2 mL of the reaction mixture was then added by syringe to a second vial containing 3-[[4-[(2R)-2-amino-4-methyl-pentoxy]-6-(2,6-dimethylphenyl)pyrimidin-2-yl]sulfamoyl]benzoic acid (50 mg, 0.1003 mmol), and acetic acid (approximately 60.23 mg, 57.04 μL, 1.003 mmol) in DCE (0.3 mL). Sodium cyanoborohydride (approximately 50.42 mg, 0.8024 mmol) was added and the reaction mixture was stirred at room temperature for an additional 2 hours. The reaction mixture was then diluted with methanol, filtered, and purified by prep HPC (1-70% ACN in water, HCl modifier, 15 min run) to give the 3-[[4-[(2R)-2-[[(4R)-2,2-dimethyl-1,3-dioxolan-4-yl]methylamino]-4-methyl-pentoxy]-6-(2,6-dimethylphenyl)pyrimidin-2-yl]sulfamoyl]benzoic acid (19 mg, 31%). ESI-MS m/z calc. 612.2618, found 613.5 (M+1)⁺; Retention time: 0.47 minutes; LC method D.

### Step 2: (11R)-12-[(2R)-2,3-Dihydroxypropyl]-6-(2,6-dimethylphenyl)-11-(2-methylpropyl)-9-oxa-2λ⁶-thia-3,5,12,19-tetraazatricyclo[12.3.1.14,8]nonadeca-1(17),4(19),5,7,14(18),15-hexaene-2,2,13-trione (Compound 38)

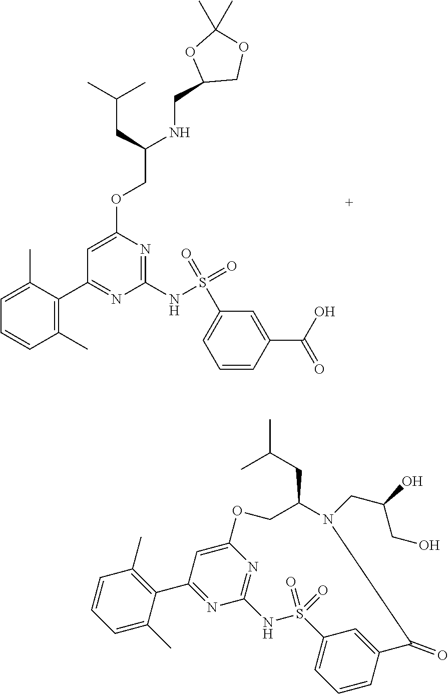 [SMILES: Cc1cccc(C)c1-c1cc(OC[C@@H](CC(C)C)NC[C@@H]2COC(C)(C)O2)nc(NS(=O)(=O)c2cccc(C(=O)O)c2)n1 ; "+" ; Cc1cccc(C)c1-c1cc2nc(n1)NS(=O)(=O)c1cccc(c1)C(=O)N(C[C@@H](O)CO)[C@H](CC(C)C)CO2]

The 3-[[4-[(2R)-2-[[(4R)-2,2-dimethyl-1,3-dioxolan-4-yl]methylamino]-4-methyl-pentoxy]-6-(2,6-dimethylphenyl)pyrimidin-2-yl]sulfamoyl]benzoic acid (hydrochloride salt) (19 mg, 0.02927 mmol) was combined with HATU (approximately 14.47 mg, 0.03805 mmol) in DMF (1 mL), and DIPEA (approximately 18.92 mg, 25.50 μL, 0.1464 mmol) was added. The reaction was stirred at room temperature for 1-2 hours, then filtered and purified by reverse phase HPLC (1-99 ACN in water, HCl modifier) to give a material that was dissolved in 2 mL 2:1 acetonitrile/1M HCl, and let stand for one hour, before concentrating and drying to give the fully deprotected diol (11R)-12-[(2R)-2,3-dihydroxypropyl]-6-(2,6-dimethylphenyl)-11-(2-methylpropyl)-9-oxa-2λ⁶-thia-3,5,12,19-tetraazatricyclo[12.3.1.14,8]nonadeca-1(17),4(19),5,7,14(18),15-hexaene-2,2,13-trione (1.1 mg, 7%). ESI-MS m/z calc. 554.2199, found 555.5 (M+1)⁺; Retention time: 1.36 minutes; LC method A.

### Example 26: Preparation of Compound 39

### Step 1: 3-[[4-[(2R)-2-[[(4S)-2,2-dimethyl-1,3-dioxolan-4-yl]methylamino]-4-methyl-pentoxy]-6-(2,6-dimethylphenyl)pyrimidin-2-yl]sulfamoyl]benzoic acid

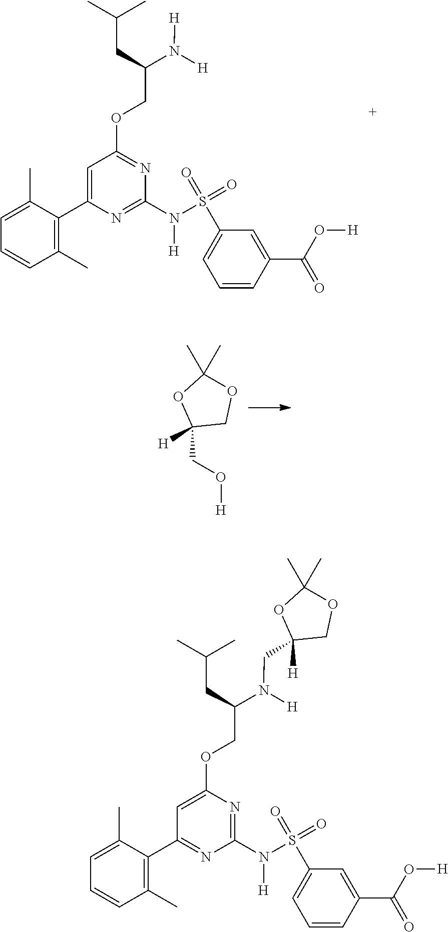

[(4S)-2,2-Dimethyl-1,3-dioxolan-4-yl]methanol (approximately 66.28 mg, 61.94 μL, 0.5015 mmol) was combined with Dess-Martin periodinane (approximately 212.7 mg, 0.5015 mmol) in DCM (0.4 mL). This reaction mixture was stirred for 30 minutes at room temperature. 0.2 mL of the reaction mixture was then added by syringe to a second vial containing 3-[[4-[(2R)-2-amino-4-methyl-pentoxy]-6-(2,6-dimethylphenyl)pyrimidin-2-yl]sulfamoyl]benzoic acid (50 mg, 0.1003 mmol), and acetic acid (approximately 60.23 mg, 57.04 μL, 1.003 mmol) in DCE (0.3 mL). Sodium cyanoborohydride (approximately 50.42 mg, 0.8024 mmol) was added and the reaction mixture was stirred at room temperature for an additional 2 hours. The reaction mixture was then diluted with methanol, filtered, and purified by prep HPC (1-70% ACN in water, HCl modifier, 15 min run) to give the 3-[[4-[(2R)-2-[[(4S)-2,2-dimethyl-1,3-dioxolan-4-yl]methylamino]-4-methyl-pentoxy]-6-(2,6-dimethylphenyl)pyrimidin-2-yl]sulfamoyl]benzoic acid (32 mg, 52%). ESI-MS m/z calc. 612.2618, found 613.5 (M+1)⁺; Retention time: 0.47 minutes; LC method D.

### Step 2: (11R)-12-[(2S)-2,3-Dihydroxypropyl]-6-(2,6-dimethylphenyl)-11-(2-methylpropyl)-9-oxa-2λ⁶-thia-3,5,12,19-tetraazatricyclo[12.3.1.14,8]nonadeca-1(17),4(19),5,7,14(18),15-hexaene-2,2,13-trione (Compound 39)

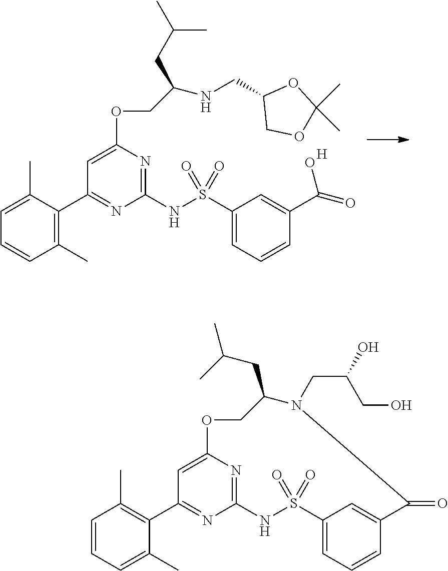

3-[[4-[(2R)-2-[[(4S)-2,2-Dimethyl-1,3-dioxolan-4-yl]methylamino]-4-methyl-pentoxy]-6-(2,6-dimethylphenyl)pyrimidin-2-yl]sulfamoyl]benzoic acid (hydrochloride salt) (17 mg, 0.02619 mmol) was combined with HATU (approximately 12.95 mg, 0.03405 mmol) in DMF (1 mL), and DIPEA (approximately 16.93 mg, 22.82 μL, 0.1310 mmol) was added. The reaction was stirred at room temperature for 1-2 hours, then filtered and purified by reverse phase HPLC (1-99% ACN in water, HCl modifier) to give a material which was dissolved in 2 mL 2:1 acetonitrile/1M HCl, and let stand for one hour, before concentrating and drying to give the fully deprotected diol (11R)-12-[(2S)-2,3-dihydroxypropyl]-6-(2,6-dimethylphenyl)-11-(2-methylpropyl)-9-oxa-2?⁶-thia-3,5,12,19-tetraazatricyclo[12.3.1.14,8]nonadeca-1(17),4(19),5,7,14(18),15-hexaene-2,2,13-trione (1.7 mg, 12%). ESI-MS m/z calc. 554.2199, found 555.5 (M+1)⁺; Retention time: 1.38 minutes; LC method A.

### Example 27: Preparation of Compound 40

### Step 1: {7-Oxaspiro[3.5]nonan-2-yl}methanol

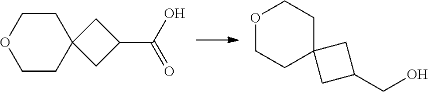 [SMILES: O=C(O)C1CC2(CCOCC2)C1 ; OCC1CC2(CCOCC2)C1]

7-Oxaspiro[3.5]nonane-2-carboxylic acid (300 mg, 1.763 mmol) was dissolved in THE (6 mL) and cooled to 0° C. Lithium Aluminum Hydride (1.2 mL of 2 M, 2.400 mmol) (in THF) was added dropwise while purging with nitrogen. The reaction mixture was then allowed to warm to room temperature and stirred for 16 hours. After this time, the reaction mixture was cooled to 0° C., diluted with 5 mL diethyl ether, then quenched with 1 mL water, followed by 3 mL 1M NaOH. The resulting suspension was warmed to room temperature, then further diluted with diethyl ether and dried using a large quantity of magnesium sulfate. The suspension was filtered, washing with ethyl acetate and concentrated to give the {7-oxaspiro[3.5]nonan-2-yl}methanol (267 mg, 97%) which was used in a later step without additional purification. ESI-MS m/z calc. 156.11504, found 157.1 (M+1)⁺; Retention time: 0.3 minutes; LC method D. ¹H NMR (400 MHz, CDCl₃) δ 3.66-3.58 (m, 4H), 3.58-3.51 (m, 2H), 2.47 (pt, J=8.4, 6.6 Hz, 1H), 1.94 (ddd, J=10.4, 8.7, 2.3 Hz, 2H), 1.67-1.60 (m, 2H), 1.60-1.49 (m, 4H), 1.45 (s, 1H).

### Step 2: 3-[[4-(2,6-Dimethylphenyl)-6-[(2R)-4-methyl-2-(7-oxaspiro[3.5]nonan-2-ylmethylamino)pentoxy]pyrimidin-2-yl]sulfamoyl]benzoic acid

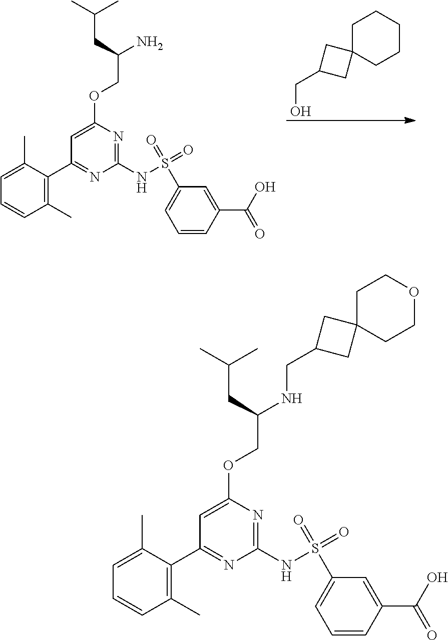 [SMILES: Cc1cccc(C)c1-c1cc(OC[C@H](N)CC(C)C)nc(NS(=O)(=O)c2cccc(C(=O)O)c2)n1 ; OCC1CC2(CCCCC2)C1 ; Cc1cccc(C)c1-c1cc(OC[C@@H](CC(C)C)NCC2CC3(CCOCC3)C2)nc(NS(=O)(=O)c2cccc(C(=O)O)c2)n1]

{7-Oxaspiro[3.5]nonan-2-yl}methanol (approximately 78.35 mg, 0.5015 mmol) was combined with Dess-Martin periodinane (approximately 212.7 mg, 0.5015 mmol) in DCM (0.4 mL). This reaction mixture was stirred for 30 minutes at room temperature. 0.2 mL of the reaction mixture was then added by syringe to a second vial containing 3-[[4-[(2R)-2-amino-4-methyl-pentoxy]-6-(2,6-dimethylphenyl)pyrimidin-2-yl]sulfamoyl]benzoic acid (50 mg, 0.1003 mmol), and acetic acid (approximately 60.23 mg, 57.04 μL, 1.003 mmol) in DCE (0.3 mL). Sodium cyanoborohydride (approximately 50.42 mg, 0.8024 mmol) was added and the reaction mixture was stirred at room temperature for an additional 2 hours. The reaction mixture was then diluted with methanol, filtered, and purified by preparative HPLC (1-70% ACN in water, HCl modifier, 15 min run) to give the 3-[[4-(2,6-dimethylphenyl)-6-[(2R)-4-methyl-2-(7-oxaspiro[3.5]nonan-2-ylmethylamino)pentoxy]pyrimidin-2-yl]sulfamoyl]benzoic acid (12.5 mg, 20%). ESI-MS m/z calc. 636.29816, found 637.5 (M+1)⁺; Retention time: 0.48 minutes; LC method D.

### Step 3: (11R)-6-(2,6-Dimethylphenyl)-11-isobutyl-12-(7-oxaspiro[3.5]nonan-2-ylmethyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 40)

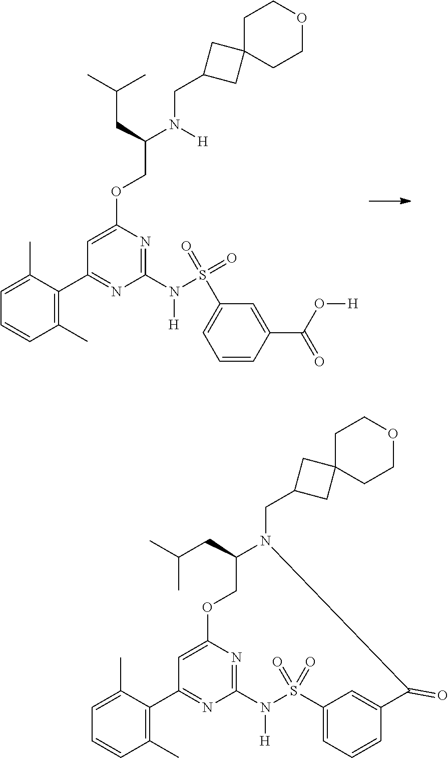 [SMILES: Cc1cccc(C)c1-c1cc(OC[C@@H](CC(C)C)NCC2CC3(CCOCC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(CC1CC3(CCOCC3)C1)[C@H](CC(C)C)CO2]

3-[[4-(2,6-Dimethylphenyl)-6-[(2R)-4-methyl-2-(7-oxaspiro[3.5]nonan-2-ylmethylamino)pentoxy]pyrimidin-2-yl]sulfamoyl]benzoic acid (12 mg, 0.01884 mmol) was combined with HATU (approximately 9.312 mg, 0.02449 mmol) in DMF (1 mL) and DIPEA (approximately 12.17 mg, 16.40 μL, 0.09420 mmol) was added. The reaction was then stirred for 1 hour. The reaction mixture was then filtered and purified by reverse phase reverse phase HPLC to give (11R)-6-(2,6-dimethylphenyl)-11-isobutyl-12-(7-oxaspiro[3.5]nonan-2-ylmethyl)-2,2-dioxo-9-oxa-2λ⁶-thia-3,5,12,19-tetrazatricyclo[12.3.1.14,8]nonadeca-1(18),4(19),5,7,14,16-hexaen-13-one (7.4 mg, 63%). ESI-MS m/z calc. 618.2876, found 619.5 (M+1)⁺; Retention time: 1.87 minutes; LC method A.

### Example 28: Preparation of Compound 41

### Step 1: 3-[[4-(2,6-Dimethylphenyl)-6-[(2R)-4-methyl-2-[(4-oxo-6,7-dihydro-5H-pyrazolo[1,5-a]pyrazin-2-yl)methylamino]pentoxy]pyrimidin-2-yl[sulfamoyl]benzoic acid

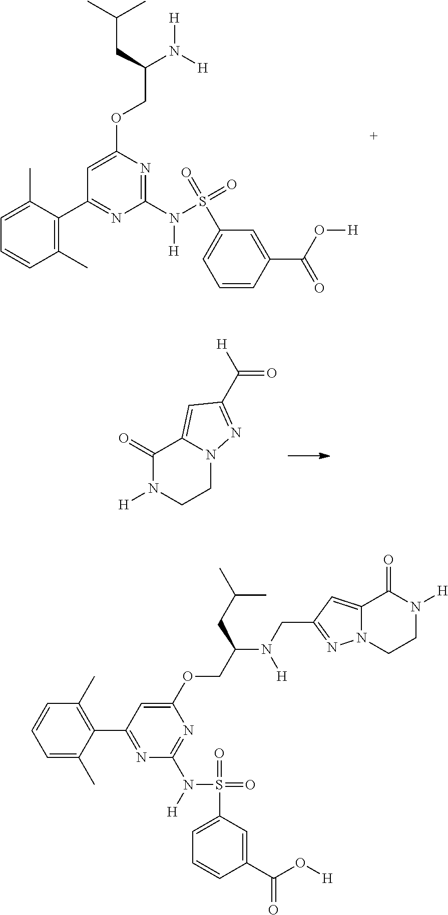 [SMILES: Cc1cccc(C)c1-c1cc(OC[C@H](N)CC(C)C)nc(NS(=O)(=O)c2cccc(C(=O)O)c2)n1 ; "+" ; O=Cc1cc2n(n1)CCNC2=O ; Cc1cccc(C)c1-c1cc(OC[C@@H](CC(C)C)NCc2cc3n(n2)CCNC3=O)nc(NS(=O)(=O)c2cccc(C(=O)O)c2)n1]

3-[[4-[(2R)-2-amino-4-methyl-pentoxy]-6-(2,6-dimethylphenyl)pyrimidin-2-yl]sulfamoyl]benzoic acid (hydrochloride salt) (50 mg, 0.09345 mmol), and 4-oxo-6,7-dihydro-5H-pyrazolo[1,5-a]pyrazine-2-carbaldehyde (approximately 46.31 mg, 0.2804 mmol) compound were combined in DCE (0.4 mL) with acetic acid (approximately 33.67 mg, 31.88 μL, 0.5607 mmol) and stirred at room temperature. After 30 minutes, sodium cyanoborohydride (approximately 23.49 mg, 0.3738 mmol) was added, and stirring at room temperature was continued for 1 hour. At this time the reaction mixture was quenched with 1 drop 1M HCl, concentrated, then diluted with DMSO/methanol (1:1) and purified by reverse phase HPLC (1-70% ACN in water HCl modifier [except as noted]) to give 3-[[4-(2,6-dimethylphenyl)-6-[(2R)-4-methyl-2-[(4-oxo-6,7-dihydro-5H-pyrazolo[1,5-a]pyrazin-2-yl)methylamino]pentoxy]pyrimidin-2-yl]sulfamoyl]benzoic acid (26.0 mg, 43%).ESI-MS m z calc. 647.2526, found 648.5 (M+1)⁺; Retention time: 0.43 minutes; LC method D.

### Step 2: (11R)-6-(2,6-dimethylphenyl)-11-isobutyl-2,2-dioxo-12-[(4-oxo-6,7-dihydro-5H-pyrazolo[1,5-a]pyrazin-2-yl)methyl]-9-oxa-2λ⁶-thia-3,5,12,19-tetrazatricyclo[12.3.1.14,8]nonadeca-1(18),4(19),5,7,14,16-hexaen-13-one (Compound 41)

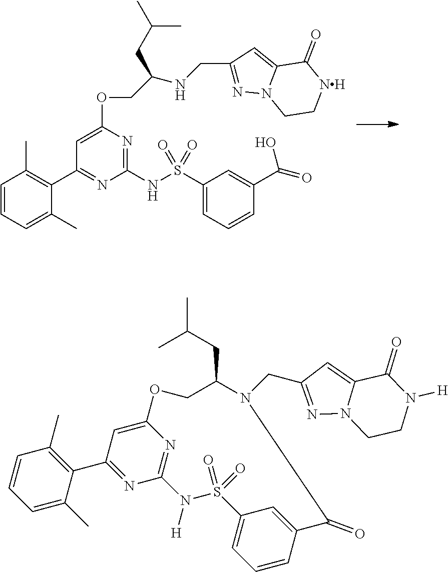

3-[[4-(2,6-Dimethylphenyl)-6-[(2R)-4-methyl-2-[(4-oxo-6,7-dihydro-5H-pyrazolo[1,5-a]pyrazin-2-yl)methylamino]pentoxy]pyrimidin-2-yl]sulfamoyl]benzoic acid (17 mg, 0.02624 mmol) was combined with HATU (approximately 11.97 mg, 0.03149 mmol) in DMSO (1 mL) and DIPEA (approximately 16.96 mg, 22.86 μL, 0.1312 mmol) was added. The reaction was stirred at room temperature for 30 minutes, then was filtered and purified by reverse phase HPLC (1-99 ACN with HCl modifier, 15 min run) to give (11R)-6-(2,6-dimethylphenyl)-11-isobutyl-2,2-dioxo-12-[(4-oxo-6,7-dihydro-5H-pyrazolo[1,5-a]pyrazin-2-yl)methyl]-9-oxa-2λ⁶-thia-3,5,12,19-tetrazatricyclo[12.3.1.14,8]nonadeca-1(18),4(19),5,7,14,16-hexaen-13-one. ESI-MS m/z calc. 629.73, found 630.5 (M+1)⁺; Retention time: 1.44 minutes; LC method A.

### Example 29: Preparation of Compound 42

### Step 1: (11R)-6-(2,6-Dimethylphenyl)-11-isobutyl-3-(methoxymethyl)-2,2-dioxo-12-[3-(trifluoromethoxy)propyl]-9-oxa-2λ⁶-thia-3,5,12,19-tetrazatricyclo[12.3.1.14,8]nonadeca-1(18),4(19),5,7,14,16-hexaen-13-one

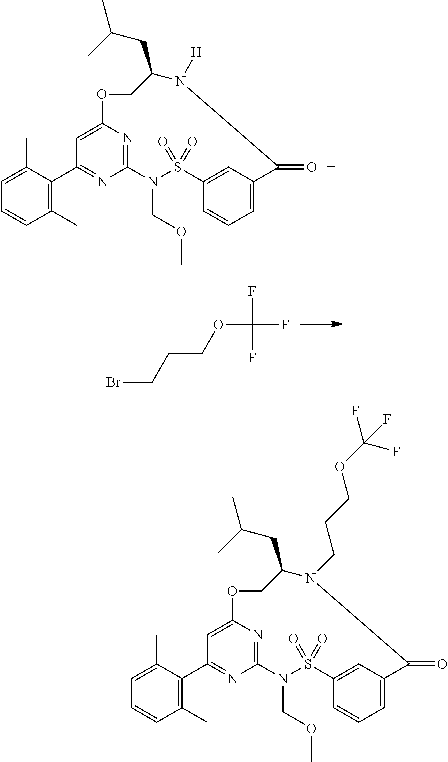

(11R)-6-(2,6-Dimethylphenyl)-11-isobutyl-3-(methoxymethyl)-2,2-dioxo-9-oxa-2?⁶-thia-3,5,12,19-tetrazatricyclo[12.3.1.14,8]nonadeca-1(18),4(19),5,7,14,16-hexaen-13-one (60 mg, 0.1144 mmol) was dissolved in DMF (1 mL) and cooled in an ice bath. Sodium hydride (15.6 mg of 60% w/w, 0.3900 mmol) was added and the reaction mixture was stirred for 20 min. 1-bromo-3-(trifluoromethoxy)propane (31 mg, 0.1498 mmol) was added and the reaction was allowed to warm to room temperature and stir for 16 h. The reaction was partitioned between ethyl acetate and a 1M HCl solution. The organics were separated, washed with brine, dried over sodium sulfate and evaporated. The crude material was purified by reverse phase HPLC utilizing a gradient of 1-99% acetonitrile in 5 mM aqueous HCl to yield (11R)-6-(2,6-dimethylphenyl)-11-isobutyl-3-(methoxymethyl)-2,2-dioxo-12-[3-(trifluoromethoxy)propyl]-9-oxa-2λ⁶-thia-3,5,12,19-tetrazatricyclo[12.3.1.14,8]nonadeca-1(18),4(19),5,7,14,16-hexaen-13-one (10.4 mg, 14%) ESI-MS m/z calc. 650.2386, found 651.4 (M+1)⁺; Retention time: 0.88 minutes; LC method D.

### Step 2: (11R)-6-(2,6-Dimethylphenyl)-11-isobutyl-2,2-dioxo-12-[3-(trifluoromethoxy)propyl]-9-oxa-2λ⁶-thia-3,5,12,19-tetrazatricyclo[12.3.1.14,8]nonadeca-1(18),4(19),5,7,14,16-hexaen-13-one (Compound 42)

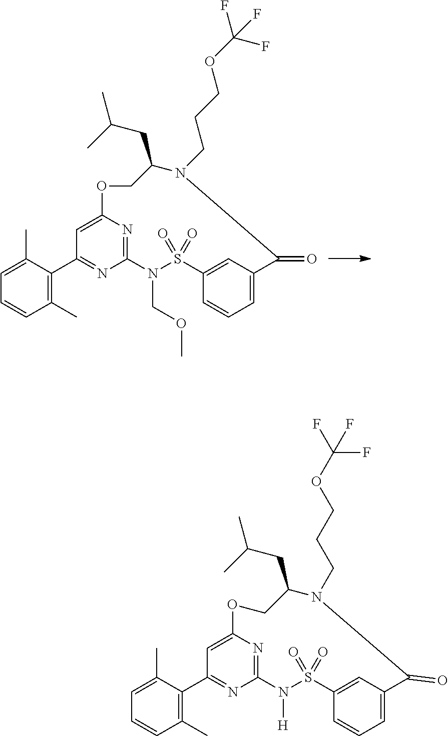 [SMILES: COCN1c2nc(cc(-c3c(C)cccc3C)n2)OC[C@@H](CC(C)C)N(CCCOC(F)(F)F)C(=O)c2cccc(c2)S1(=O)=O ; Cc1cccc(C)c1-c1cc2nc(n1)NS(=O)(=O)c1cccc(c1)C(=O)N(CCCOC(F)(F)F)[C@H](CC(C)C)CO2]

(11R)-6-(2,6-Dimethylphenyl)-11-isobutyl-3-(methoxymethyl)-2,2-dioxo-12-[3-(trifluoromethoxy)propyl]-9-oxa-2λ⁶-thia-3,5,12,19-tetrazatricyclo[12.3.1.14,8]nonadeca-1(18),4(19),5,7,14,16-hexaen-13-one (10.4 mg, 0.01598 mmol) was dissolved in a mixture of TFA (0.2 mL, 2.596 mmol) and DCM (1 mL) and stirred at room temperature for 1 h. The reaction was evaporated and the resulting material was purified by reverse phase HPLC utilizing a gradient of 1-99% acetonitrile in 5 mM aqueous HCl to yield (11R)-6-(2,6-dimethylphenyl)-11-isobutyl-2,2-dioxo-12-[3-(trifluoromethoxy)propyl]-9-oxa-2?⁶-thia-3,5,12,19-tetrazatricyclo[12.3.1.14,8]nonadeca-1(18),4(19),5,7,14,16-hexaen-13-one (6.7 mg, 65%) ESI-MS m/z calc. 606.2124, found 607.4 (M+1)⁺; Retention time: 1.96 minutes; LC method A.

### Example 30: Preparation of Compound 43

### Step 1: Methyl 3-[[4-chloro-6-(2,6-dimethylphenyl)pyrimidin-2-yl]-(methoxymethyl)sulfamoyl]benzoate

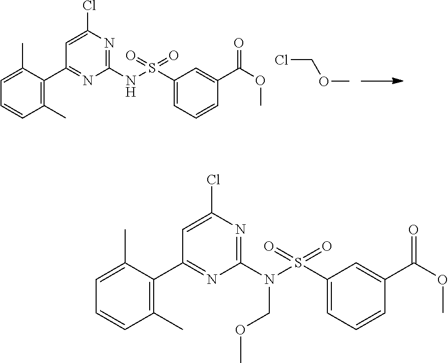

To a solution of methyl 3-[[4-chloro-6-(2,6-dimethylphenyl)pyrimidin-2-yl]sulfamoyl]benzoate (68.5 g, 158.60 mmol) in DMF (400 mL) at 0° C. was added potassium carbonate (44 g, 318.37 mmol) and chloro(methoxy)methane (13.992 g, 13.2 mL, 173.78 mmol). The reaction was stirred at room temperature for 1 h. Water (800 mL) was added and the product was extracted with DCM (3×150 mL). Combined organic layers were washed with a 1:1 mix of water and brine (4×200 mL), and then brine (1×150 mL). The resulting combined organic layers were dried over sodium sulfate, filtered and concentrated under reduced pressure. Afforded methyl 3-[[4-chloro-6-(2,6-dimethylphenyl)pyrimidin-2-yl]-(methoxymethyl)sulfamoyl]benzoate (80.4 g, 90%) as a brown oil. ESI-MS m/z calc. 475.09686, found 476.2 (M+1)⁺; Retention time: 2.06 minutes; LC method X.

### Step 2: 3-[[4-Chloro-6-(2,6-dimethylphenyl)pyrimidin-2-yl]-(methoxymethyl)sulfamoyl]benzoic acid

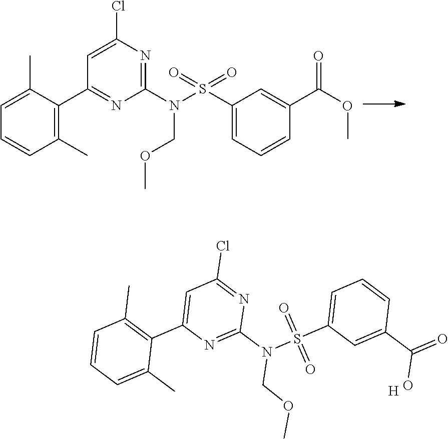

A mixture of methyl 3-[[4-chloro-6-(2,6-dimethylphenyl)pyrimidin-2-yl]-(methoxymethyl)sulfamoyl]benzoate (47.89 g, 80.698 mmol) in THE (475 mL) and water (475 mL) was treated with lithium hydroxide hydrate (5.07 g, 120.82 mmol) and it was stirred at room temperature for 4 hours. Most of the THF was removed under reduced pressure, and the remaining aqueous layer was acidified to a pH of about 2-3 using 1N aqueous HCl (250 ml). The product was extracted with ethyl acetate (3×450 mL). The combined organic layers were washed with brine (100 mL), dried over sodium sulfate, filtered and concentrated under reduced pressure. The resulting sticky solid was triturated twice in ethyl acetate (100 ml and 75 ml) to afford 3-[[4-chloro-6-(2,6-dimethylphenyl)pyrimidin-2-yl]-(methoxymethyl)sulfamoyl]benzoic acid (26.045 g, 65%) as a white solid. ¹H NMR (400 MHz, DMSO-d₆) δ 13.37 (br. s., 1H), 8.48 (s, 1H), 8.20-8.10 (m, 2H), 7.61 (t, J 7.8 Hz, 1H), 7.44 (s, 1H), 7.28-7.20 (m, 1H), 7.10 (d, J 7.6 Hz, 2H), 5.61 (s, 2H), 3.30 (s, 3H), 1.84 (s, 6H). ESI-MS m/z calc. 461.0812, found 462.1 (M+1)⁺; Retention time: 4.32 minutes; LC method Y.

### Step 3: 3-[[4-[(2R)-2-Amino-4-methyl-pentoxy]-6-(2,6-dimethylphenyl)pyrimidin-2-yl]-(methoxymethyl)sulfamoyl]benzoic acid

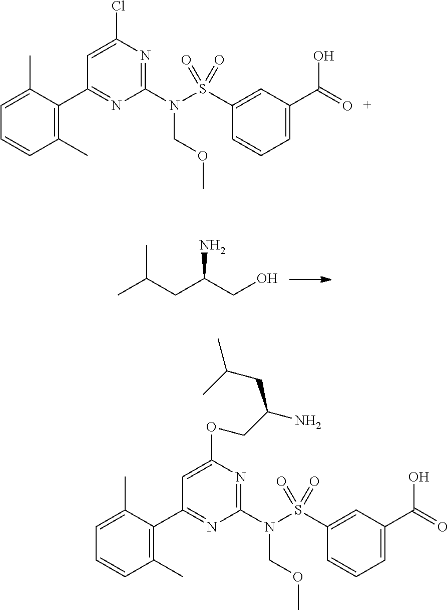

In a reaction vial, 3-[[4-chloro-6-(2,6-dimethylphenyl)pyrimidin-2-yl]-(methoxymethyl)sulfamoyl]benzoic acid (2.6 g, 5.629 mmol), (2R)-2-amino-4-methyl-pentan-1-ol (725 μL, 5.673 mmol), and sodium tert-butoxide (1.75 g, 18.21 mmol) were combined in THE (7 mL) and stirred at room temperature for 2 h. The reaction was diluted with ethyl acetate and washed with a 1M HCl solution. The organics were further washed with brine, dried over sodium sulfate and evaporated. The crude material was recrystallized from ethyl acetate to provide the product as a white solid 3-[[4-[(2R)-2-amino-4-methyl-pentoxy]-6-(2,6-dimethylphenyl)pyrimidin-2-yl]-(methoxymethyl)sulfamoyl]benzoic acid (hydrochloride salt)(1.95 g, 60%) ESI-MS m/z calc. 542.2199, found 543.3 (M+1)⁺; Retention time: 1.4 minutes (LC method A).

### Step 4: (11R)-6-(2,6-Dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

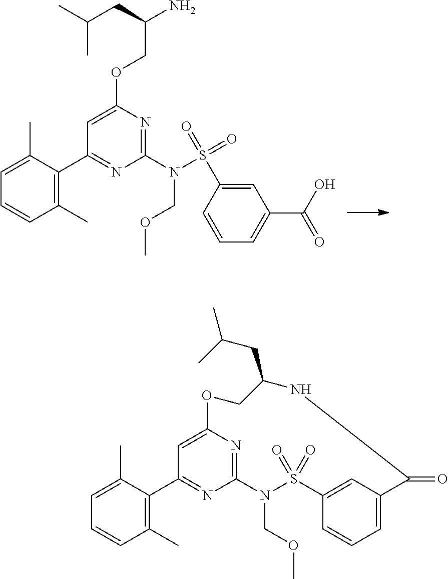 [SMILES: COCN(c1nc(OC[C@H](N)CC(C)C)cc(-c2c(C)cccc2C)n1)S(=O)(=O)c1cccc(C(=O)O)c1 ; COCN1c2nc(cc(-c3c(C)cccc3C)n2)OC[C@@H](CC(C)C)NC(=O)c2cccc(c2)S1(=O)=O]

3-[[4-[(2R)-2-amino-4-methyl-pentoxy]-6-(2,6-dimethylphenyl)pyrimidin-2-yl]-(methoxymethyl)sulfamoyl]benzoic acid (hydrochloride salt) (797 mg, 1.376 mmol) was dissolved in DMF (6 mL) and added to a solution of HATU (640.2 mg, 1.684 mmol) and triethylamine (766 μL, 5.496 mmol) in DMF (7 mL). The reaction was stirred at room temperature for 20 min. The reaction mixture was poured into water (20 mL) and the resulting solid was collected via filtration. The solids were dissolved in ethyl acetate and washed with a 1M HCl solution, then brine. The organics were dried over sodium sulfate and evaporated to give (11R)-6-(2,6-dimethylphenyl)-11-isobutyl-3-(methoxymethyl)-2,2-dioxo-9-oxa-2?⁶-thia-3,5,12,19-tetrazatricyclo[12.3.1.14,8]nonadeca-1(18),4(19),5,7,14,16-hexaen-13-one (720 mg, 100%) ESI-MS m/z calc. 524.20935, found 525.3 (M+1)⁺; Retention time: 0.77 minutes; LC method D.

### Step 5: Ethyl 2-[(11R)-6-(2,6-dimethylphenyl)-11-isobutyl-3-(methoxymethyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]acetate

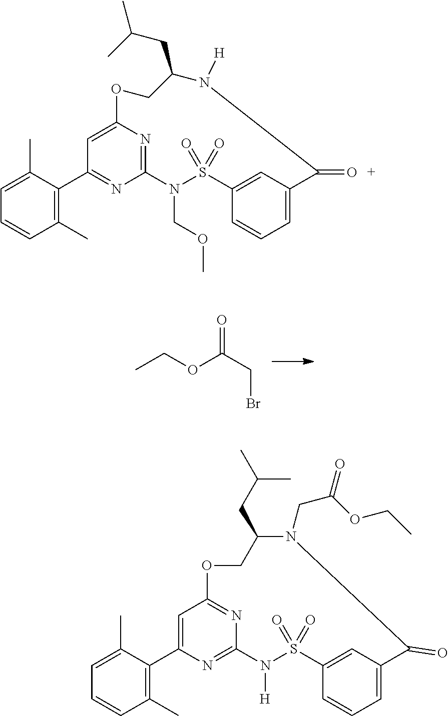

In a reaction vial, (11R)-6-(2,6-dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (135 mg, 0.2573 mmol) was dissolved in DMF (700 μL) along with sodium hydride (20.6 mg of 60% w/w, 0.5150 mmol) and stirred at rt for 30 min. To the reaction mixture, ethyl 2-bromoacetate (64.5 mg, 0.3862 mmol) and sodium(1+) (Iodide Ion (1)) (7.7 mg, 0.05137 mmol) were added. The reaction was allowed to stir at rt for 2 h. The reaction was quenched with ethanol then partitioned between ethyl acetate and saturated NaCl solution. The organic layer was isolated, dried over anhydrous sodium sulfate, filtered, and evaporated to dryness. The crude material was purified by column chromatography on silica using 10-80% ethyl acetate/hexanes gradient. The product was recovered as an off-white solid. Ethyl 2-[(11R)-6-(2,6-dimethylphenyl)-11-isobutyl-3-(methoxymethyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]acetate (49.7 mg, 32%)¹H NMR (400 MHz, Methanol-d4) δ 8.82 (t, J 1.8 Hz, 1H), 8.20 (dt, J 7.8, 1.5 Hz, 1H), 7.83 (dt, J 7.6, 1.4 Hz, 1H), 7.77 (t, J 7.7 Hz, 1H), 7.19 (dd, J 8.2, 7.0 Hz, 1H), 7.09 (d, J 7.6 Hz, 2H), 6.51 (s, 1H), 5.84 (d, J 10.8 Hz, 1H), 5.66 (d, J 10.8 Hz, 1H), 5.33-5.26 (m, 1H), 4.36 (d, J 17.1 Hz, 1H), 4.28 (dd, J 7.2, 4.0 Hz, 1H), 4.26-4.21 (m, 1H), 4.21-4.09 (m, 3H), 3.10 (s, 3H), 2.02 (s, 5H), 1.68 (ddd, J=13.3, 9.9, 3.0 Hz, 1H), 1.44 (ddd, J=9.8, 6.5, 3.2 Hz, 1H), 1.34 (t, J 7.1 Hz, 5H), 0.78 (d, J 6.5 Hz, 3H), 0.30 (d, J 6.3 Hz, 3H). ESI-MS m/z calc. 610.2461, found 611.3 (M+1)⁺; Retention time: 2.17 minutes (LC method A).

### Step 6: (11R)-6-(2,6-Dimethylphenyl)-12-(2-hydroxy-2-methyl-propyl)-11-isobutyl-2,2-dioxo-9-oxa-2λ⁶-thia-3,5,12,19-tetrazatricyclo[12.3.1.14,8]nonadeca 1(18),4(19),5,7,14,16-hexaen-13-one (Compound 43)

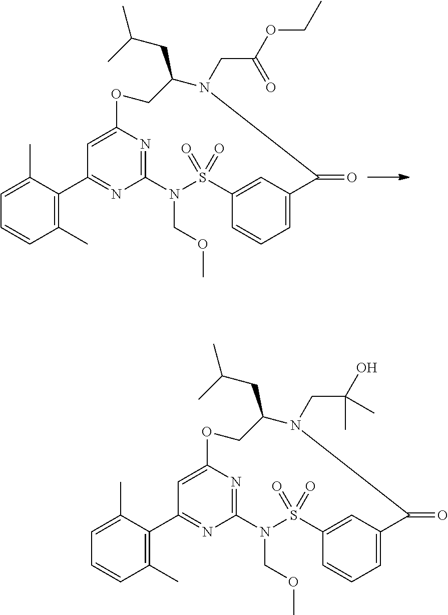 [SMILES: CCOC(=O)CN1C(=O)c2cccc(c2)S(=O)(=O)N(COC)c2nc(cc(-c3c(C)cccc3C)n2)OC[C@H]1CC(C)C ; COCN1c2nc(cc(-c3c(C)cccc3C)n2)OC[C@@H](CC(C)C)N(CC(C)(C)O)C(=O)c2cccc(c2)S1(=O)=O]

In a reaction vial, bromo(methyl)magnesium (35.4 μL, 0.3058 mmol) was added dropwise to a solution of ethyl 2-[(11R)-6-(2,6-dimethylphenyl)-11-isobutyl-3-(methoxymethyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]acetate (44.7 mg, 0.07319 mmol) in THE (510 μL) at 0° C. The reaction was allowed to stir at 0° C. for 5 min then warmed to rt overnight. The reaction mixture was quenched with saturated ammonium chloride solution and extracted with ethyl acetate. The organic layer was isolated, dried over anhydrous sodium sulfate, filtered, and evaporated to dryness. The crude material was dissolved in DCM (500 μL) along with TFA (1 mL, 12.98 mmol) and stirred at rt for 1.5 h. The reaction was evaporated to dryness and purified by preparative HPLC to give as a white solid (11R)-6-(2,6-dimethylphenyl)-12-(2-hydroxy-2-methyl-propyl)-11-isobutyl-2,2-dioxo-9-oxa-2λ⁶-thia-3, 5,12,19-tetrazatricyclo[12.3.1.14,8]nonadeca-1(18),4(19),5,7,14,16-hexaen-13-one (16.62 mg, 41%)¹H NMR (400 MHz, DMSO-d₆) δ 13.01 (s, 1H), 8.54 (s, 1H), 8.02-7.83 (m, 1H), 7.78-7.54 (m, 3H), 7.26 (t, J=7.7 Hz, 1H), 7.12 (d, J=7.6 Hz, 2H), 6.34 (s, 1H), 5.20 (dd, J=11.3, 4.1 Hz, 1H), 4.78 (s, 1H), 4.50 (t, J=11.3 Hz, 1H), 4.01 (d, J=14.2 Hz, 1H), 3.91 (t, J=11.3 Hz, 1H), 2.90 (d, J=14.3 Hz, 1H), 1.70 (t, J=11.0 Hz, 2H), 1.23 (s, 6H), 1.13 (s, 4H), 0.74 (d, J=5.6 Hz, 4H), 0.28 (d, J=5.3 Hz, 4H). ESI-MS m/z calc. 552.24066, found 553.3 (M+1)⁺; Retention time: 1.67 minutes (LC method A).

### Example 31: Preparation of Compound 44

### Step 1: tert-Butyl (4S)-2-(hydroxymethyl)-4-phenyl-oxazolidine-3-carboxylate

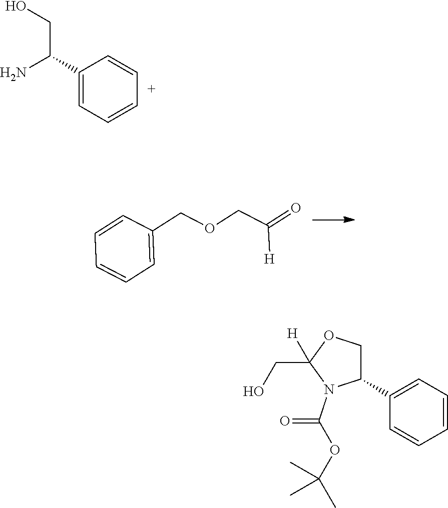 [SMILES: N[C@H](CO)c1ccccc1 ; O=CCOCc1ccccc1 ; CC(C)(C)OC(=O)N1C(CO)OC[C@@H]1c1ccccc1]

In a 100-mL round-bottomed flask, (2S)-2-amino-2-phenyl-ethanol (1.7925 g, 12.81 mmol) was dissolved in dry DCM (40 mL), to which 2-benzyloxyacetaldehyde (1.80 mL, 12.81 mmol) and anhydrous sodium sulfate (3.31 g, 23.30 mmol) were added. This mixture was stirred vigorously at room temperature for 25 h. After this time, TEA (5.0 mL, 35.87 mmol) and Boc anhydride (3.31 g, 15.17 mmol) were added, followed by DMAP (10.5 mg, 0.08595 mmol). This mixture was stirred at room temperature for 2 h, after which a second portion of Boc anhydride (3.31 g, 15.17 mmol) was added and stirred for an additional 13 h. Thereafter, it was filtered over a fritted funnel and evaporated in vacuo to give a yellow liquid. This crude product was purified by silica gel chromatography (120 g of silica, 0 to 30% gradient of ethyl acetate/hexanes) to give tert-butyl (4S)-2-(benzyloxymethyl)-4-phenyl-oxazolidine-3-carboxylate (2.2503 g, 30%) ESI-MS m/z calc. 369.194, found 370.3 (M+1)⁺; Retention time: 2.05 minutes, LC method A.

In a 100-mL round-bottomed flask, the impure product was dissolved in EtOH (40 mL). This solution was sparged with a balloon of hydrogen gas for 5 min. The cap was briefly removed, and Pd(OH)₂/C (1.256 g of 10% w/w, 0.8944 mmol) was added. This reaction mixture was stirred under hydrogen (2 L, 79.37 mmol) at room temperature for 103 h, after which it was filtered through Celite and rinsed with methanol (80 mL). This solution was evaporated in vacuo to give a viscous oil, tert-butyl (4S)-2-(hydroxymethyl)-4-phenyl-oxazolidine-3-carboxylate (1.1509 g, 26%) ESI-MS m/z calc. 279.14706, found 280.2 (M+1)⁺; Retention time: 1.39 minutes, LC method A.

### Step 2: 3-[[4-[[(4S)-3-tert-Butoxycarbonyl-4-phenyl-oxazolidin-2-yl]methoxy]-6-(2,6-dimethylphenyl)pyrimidin-2-yl]sulfamoyl]benzoic acid

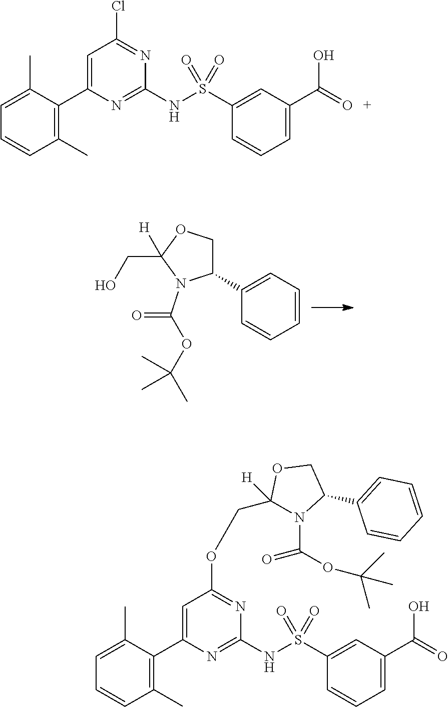

In a 100-mL round-bottomed flask, 3-[[4-chloro-6-(2,6-dimethylphenyl)pyrimidin-2-yl]sulfamoyl]benzoic acid (2.3023 g, 5.510 mmol) and tert-butyl (4S)-2-(hydroxymethyl)-4-phenyl-oxazolidine-3-carboxylate (1.1509 g, 3.708 mmol) were dissolved in NMP (20 mL), and this solution was cooled to 0° C. NaH (0.9031 g of 60% w/w, 22.58 mmol) was added in one portion (CAUTION: evolution of gas and heat), and this mixture was stirred at 0° C. for 5 min then at 50° C. for 15 min. It was then quenched by pouring onto 1 N HCl solution (25 mL), then was extracted with ethyl acetate (3×50 mL). The combined organic extracts were washed with water (100 mL) and saturated aqueous sodium chloride solution (100 mL), then dried over sodium sulfate, filtered, and evaporated in vacuo to give 3 g of a yellow oil. This crude product was purified by silica gel chromatography (120 g of silica, 0 to 80% gradient of ethyl acetate/hexanes) to give a white foam, 3-[[4-[[(4S)-3-tert-butoxycarbonyl-4-phenyl-oxazolidin-2-yl]methoxy]-6-(2,6-dimethylphenyl)pyrimidin-2-yl]sulfamoyl]benzoic acid (4.9812 g, 77%) ESI-MS m/z calc. 660.2254, found 661.4 (M+1)⁺; Retention time: 1.88 minutes, LC method A.

### Step 3: 3-[[4-(2,6-Dimethylphenyl)-6-(2-oxoethoxy)pyrimidin-2-yl]sulfamoyl]benzoic acid

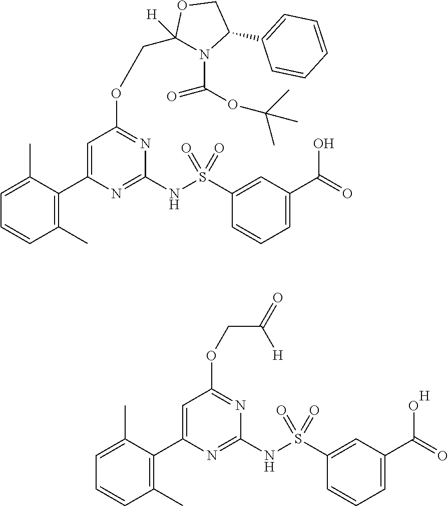

In a 50-mL round-bottomed flask, 3-[[4-[[(4S)-3-tert-butoxycarbonyl-4-phenyl-oxazolidin-2-yl]methoxy]-6-(2,6-dimethylphenyl)pyrimidin-2-yl]sulfamoyl]benzoic acid (4.9812 g, 2.865 mmol) was dissolved in dioxane (12.0 mL), to which a dioxane solution of HCl (4.0 mL of 4.0 M, 16.00 mmol) was added. This solution was stirred at 70° C. for 30 min, after which it was cooled to room temperature and evaporated to dryness in vacuo. This crude product was purified by silica gel chromatography (120 g of silica, 0 to 100% gradient of ethyl acetate/hexanes) to give a white foam, 3-[[4-(2,6-dimethylphenyl)-6-(2-oxoethoxy)pyrimidin-2-yl]sulfamoyl]benzoic acid (0.9135 g, 72%) ESI-MS m/z calc. 441.09946, found 442.3 (M+1)⁺; Retention time: 1.08 minutes. Note: An (M+ water+H)⁺ mass of 460.3 is more prominent, LC method A.

### Step 4: 12-Benzyl-N-tert-butyl-6-(2,6-dimethylphenyl)-2,2,13-trioxo-9-oxa-2⁶-thia-3,5,12,19-tetrazatricyclo[12.3.1.14,8]nonadeca-1(18),4(19),5,7,14,16-hexaene-11-carboxamide (Compound 44)

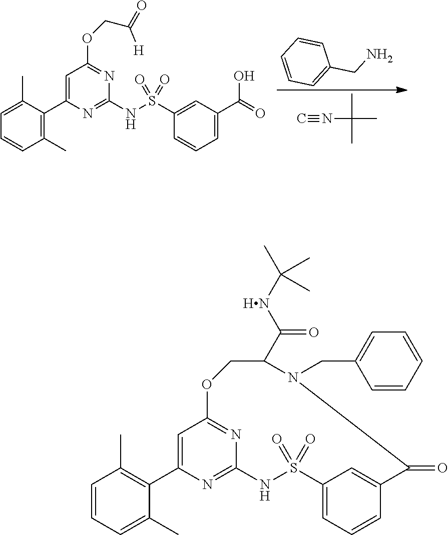

In a 1-mL vial, 3-[[4-(2,6-dimethylphenyl)-6-(2-oxoethoxy)pyrimidin-2-yl]sulfamoyl]benzoic acid (20.0 mg, 0.04530 mmol) was dissolved in MeOH (300 μL), to which benzylamine (4.9 mg, 0.04573 mmol) (0.046 mmol) and t-butyl isocyanide (3.8 mg, 0.04571 mmol) (0.046 mmol) were added in this order. This mixture was stirred at room temperature for 16 h, after which it was diluted with MeOH (500 μL), filtered, and purified by reverse phase preparative chromatography using a C₁₈ column and a gradient eluent of 1 to 99% acetonitrile in water containing 5 mM hydrochloric acid to give almost pure but colored products. The compound was re-purified by preparative TLC (one-sixth of a full silica plate-20 cm×20 cm, 250 m thickness, 60 Å particle size—with 75% ethyl acetate/hexanes, UV active band) to give 12-benzyl-N-tert-butyl-6-(2,6-dimethylphenyl)-2,2,13-trioxo-9-oxa-2λ⁶-thia-3,5,12,19-tetrazatricyclo[12.3.1.14,8]nonadeca-1(18),4(19),5,7,14,16-hexaene-11-carboxamide (1 mg, 4%). ESI-MS m/z calc. 613.2359, found 614.4 (M+1)⁺; Retention time: 2.33 minutes; LC method A.

### Example 32: Preparation of Compound 45

### Step 1: tert-Butyl N-[(1R)-1-[[6-(2,6-dimethylphenyl)-2-[[3-[methoxy(methyl)carbamoyl]phenyl]sulfonylamino]pyrimidin-4-yl]oxymethyl]-3-methyl-butyl]carbamate

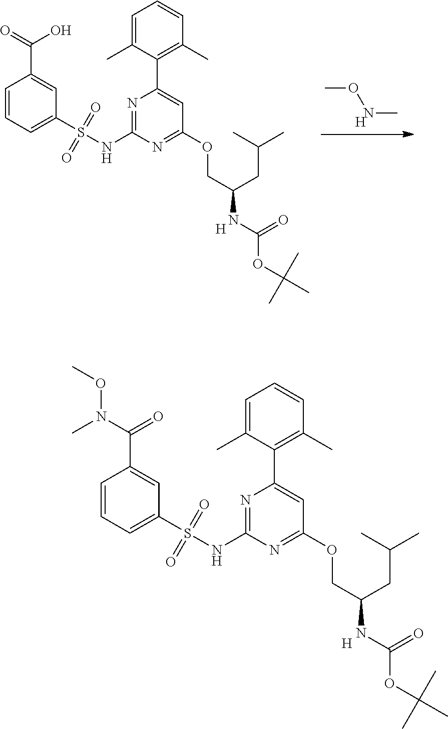

To a solution of 3-[[4-[(2R)-2-(tert-butoxycarbonylamino)-4-methyl-pentoxy]-6-(2,6-dimethylphenyl)pyrimidin-2-yl]sulfamoyl]benzoic acid (crude 67%, 6.6 g, 7.38 mmol) in DMF (40 mL) were added N,O-dimethylhydroxylamine (hydrochloride salt) (1.62 g, 16.608 mmol), DIPEA (4.3 g, 5.7951 mL, 33.271 mmol) and HATU (6.3 g, 16.569 mmol). The mixture was stirred at rt overnight. Ice-water (80 g) was added. The mixture was extracted with DCM. The organic phase was concentrated and the residue was purified by flash chromatography (120 g silica gel, heptanes/EtOAc 30-50%) to afford tert-butyl N-[(1R)-1-[[6-(2,6-dimethylphenyl)-2-[[3-[methoxy(methyl)carbamoyl]phenyl]sulfonylamino]pyrimidin-4-yl]oxymethyl]-3-methyl-butyl]carbamate (5 g, 80% purity, 84%) as pale-yellow oil. ¹H NMR (300 MHz, CDCl₃) δ 0.92-0.95 (m, 6H), 1.31-1.37 (m, 2H), 1.42 (s, 9H), 1.66-1.74 (m, 1H), 2.09 (s, 6H), 3.36 (s, 3H), 3.51 (s, 3H), 3.98-4.09 (m, 1H), 4.11-4.29 (m, 2H), 4.44-4.53 (m, 1H), 6.12 (s, 1H), 7.03-7.12 (m, 2H), 7.18-7.25 (m, 1H), 7.52 (t, J 7.9 Hz, 1H), 7.84 (d, J 7.9 Hz, 1H), 8.09 (d, J 7.9 Hz, 1H), 8.40 (s, 1H), 9.80 (br. s, 1H). ESI-MS m/z calc. 641.2883, found 642.3 (M+1)⁺; Retention time: 2.23 minutes, LC method K.

### Step 2: tert-Butyl N-[(1R)-1-[[6-(2,6-dimethylphenyl)-2-[(3-formylphenyl)sulfonylamino]pyrimidin-4-yl]oxymethyl]-3-methyl-butyl]carbamate

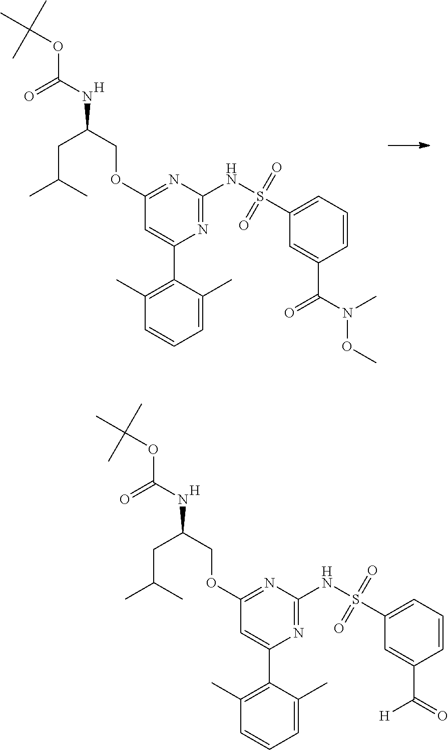

To a solution of tert-butyl N-[(1R)-1-[[6-(2,6-dimethylphenyl)-2-[[3-[methoxy(methyl)carbamoyl]phenyl]sulfonylamino]pyrimidin-4-yl]oxymethyl]-3-methyl-butyl]carbamate (282 mg, 0.4394 mmol) in DCM (35 mL) at −78° C. was added DIBAL (1.3 mL of 1 M in DCM, 1.3000 mmol). The mixture was stirred at −78° C. for 1 h. More DIBAL (1 mL of 1 M in DCM, 1.0000 mmol) was added. The mixture was stirred at −78° C. for 1 h. More DIBAL (0.5 mL of 1 M in DCM, 0.5000 mmol) was added and the mixture was stirred at −78° C. for 1 h. EtOAc (5 mL) was added. The mixture was allowed to warm up to rt slowly and stirred for 10 min. The mixture was cooled with ice-water bath. Water (0.5 mL) was added. The mixture was stirred at rt for 10 min. Sodium sulfate (5 g) was added. The mixture was stirred at rt overnight, filtered and washed with EtOAc. The filtrate was purified by flash chromatography (40 g silica gel, heptanes/EtOAc 0-50%) to afford tert-butyl N-[(1R)-1-[[6-(2,6-dimethylphenyl)-2-[(3-formylphenyl)sulfonylamino]pyrimidin-4-yl]oxymethyl]-3-methyl-butyl]carbamate (217 mg, 85%) as a white solid. ¹H NMR (300 MHz, CDCl₃) δ 0.91-1.00 (m, 6H), 1.31-1.48 (m, 11H), 1.64-1.77 (m, 1H), 2.08 (s, 6H), 3.89-4.11 (m, 1H), 4.13-4.37 (m, 2H), 4.46-4.66 (m, 1H), 6.15 (s, 1H), 7.09 (d, J 7.6 Hz, 2H), 7.22-7.29 (m, 1H), 7.65 (t, J 7.5 Hz, 1H), 8.05 (d, J 7.6 Hz, 1H), 8.28 (d, J=7.6 Hz, 1H), 8.56 (s, 1H), 9.51-9.88 (m, 1H), 9.95 (s, 1H). ESI-MS m/z calc. 582.2512, found 583.3 (M+1)⁺; Retention time: 2.26 minutes, LC method K.

### Step 3: tert-Butyl N-[(1R)-1-[[6-(2,6-dimethylphenyl)-2-[[3-(hydroxymethyl)phenyl]sulfonylamino]pyrimidin-4-yl]oxymethyl]-3-methyl-butyl]carbamate

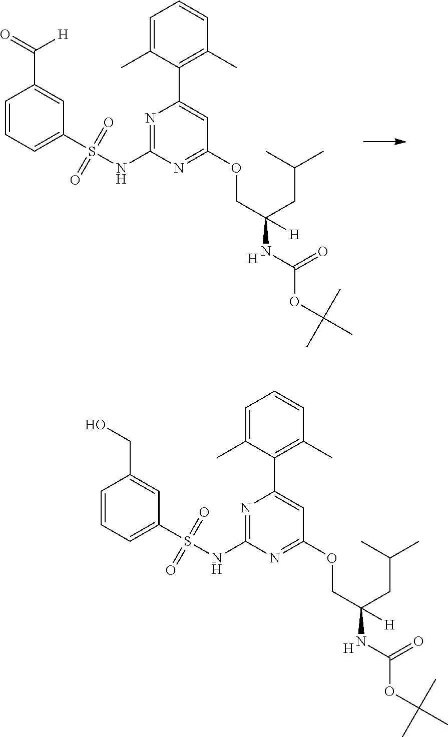

To a solution of tert-butyl N-[(1R)-1-[[6-(2,6-dimethylphenyl)-2-[(3-formylphenyl)sulfonylamino]pyrimidin-4-yl]oxymethyl]-3-methyl-butyl]carbamate (4.35 g, 7.4651 mmol) in THE (100 mL) at −70° C. was added LAH (5.5 mL of 1 M in THF, 5.5000 mmol) dropwise. The mixture was warmed up to −20° C. and stirred at −20° C. to −15° C. for 15 min. A gel was formed. Saturated ammonium chloride (100 mL) was added. The mixture was stirred at rt for 40 min and extracted with EtOAc. The organic layer was dried with sodium sulfate. Flash chromatography (120 g silica gel, heptanes/EtOAc 20-50%) afforded tert-butyl N-[(1R)-1-[[6-(2,6-dimethylphenyl)-2-[[3-(hydroxymethyl)phenyl]sulfonylamino]pyrimidin-4-yl]oxymethyl]-3-methyl-butyl]carbamate (3 g, 69%) as white solid. ¹H NMR (300 MHz, CDCl₃) δ 0.91-0.99 (m, 6H), 1.32-1.50 (m, 11H), 1.64-1.73 (m, 1H), 2.06 (s, 6H), 3.90-4.04 (m, 1H), 4.11-4.35 (m, 2H), 4.59-4.80 (m, 3H), 6.16 (s, 1H), 7.02-7.12 (m, 2H), 7.16-7.25 (m, 1H), 7.39-7.48 (m, 1H), 7.49-7.58 (m, 1H), 7.79 (d, J 7.6 Hz, 1H), 8.25 (br. s., 1H), 9.21 (br. s., 1H). ESI-MS m/z calc. 584.2669, found 585.3 (M+1)⁺; Retention time: 2.18 minutes, LC method K.

### Step 4:[3-[[4-[(2R)-2-(tert-Butoxycarbonylamino)-4-methyl-pentoxy]-6-(2,6-dimethylphenyl)pyrimidin-2-yl]sulfamoyl]phenyl]methyl methanesulfonate

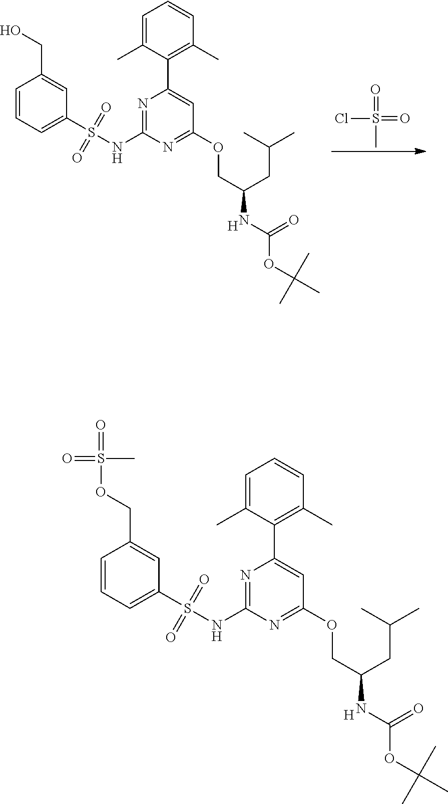

To a solution of tert-butyl N-[(1R)-1-[[6-(2,6-dimethylphenyl)-2-[[3-(hydroxymethyl)phenyl]sulfonylamino]pyrimidin-4-yl]oxymethyl]-3-methyl-butyl]carbamate (1 g, 1.7102 mmol) and TEA (347 mg, 3.4292 mmol) in DCM (25 mL) at −20° C. was added MsCl (236 mg, 2.0602 mmol) dropwise. The mixture was warmed up to −3° C. and stirred at −5 to −1° C. for 20 min. Ice-water (20 mL) was added. The mixture was extracted with DCM. The organic layer was dried with sodium sulfate. Flash chromatography (40 g silica gel, heptanes/EtOAc 20-90%) afforded [3-[[4-[(2R)-2-(tert-butoxycarbonylamino)-4-methyl-pentoxy]-6-(2,6-dimethylphenyl)pyrimidin-2-yl]sulfamoyl]phenyl]methyl methanesulfonate (0.9 g, 79%) as a white solid. ¹H NMR (300 MHz, CDCl₃) δ 0.91-1.01 (m, 6H), 1.28-1.51 (m, 11H), 1.62-1.71 (m, 1H), 2.04 (s, 6H), 3.00 (s, 3H), 3.90-4.06 (m, 1H), 4.07-4.21 (m, 2H), 4.61 (d, J 8.8 Hz, 1H), 5.10-5.28 (m, 2H), 6.10 (s, 1H), 7.02-7.08 (m, 2H), 7.16-7.25 (m, 1H), 7.43-7.65 (m, 2H), 7.97-8.18 (m, 2H), 10.58 (br. s., 1H). ESI-MS m/z calc. 662.2444, found 663.3 (M+1)⁺; Retention time: 2.25 minutes, LC method K.

### Step 5: tert-Butyl (11R)-6-(2,6-dimethylphenyl)-11-(2-methylpropyl)-2,2-dioxo-9-oxa-2λ⁶-thia-3,5,12,19-tetraazatricyclo[12.3.1.14,8]nonadeca-1(17),4,6,8(19),14(18),15-hexaene-12-carboxylate

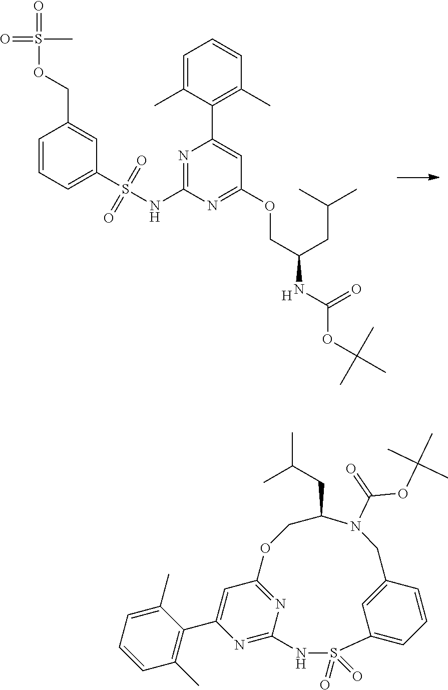

To a solution of [3-[[4-[(2R)-2-(tert-butoxycarbonylamino)-4-methyl-pentoxy]-6-(2,6-dimethylphenyl)pyrimidin-2-yl]sulfamoyl]phenyl]methyl methanesulfonate (26 mg, 0.0392 mmol) in DMF (10 mL) at 0° C. was added NaH (7 mg, 60% in mineral oil, 0.1750 mmol). The mixture was stirred at rt for 1 h. Ammonium chloride (11 mg, 6 eq) in water (0.5 mL) was added. The mixture was concentrated to remove DMF. The residue was dissolved in EtOAc and washed with water. The organic layer was dried with sodium sulfate. Flash chromatography (24 g silica gel, heptanes/EtOAc 20-40%) afforded tert-butyl (11R)-6-(2,6-dimethylphenyl)-11-(2-methylpropyl)-2,2-dioxo-9-oxa-2λ⁶-thia-3,5,12,19-tetraazatricyclo[12.3.1.14,8]nonadeca-1(17),4,6,8(19),14(18),15-hexaene-12-carboxylate (11 mg, 50%) as white solid. ¹H NMR (300 MHz, CDCl₃) δ 0.76-0.84 (m, 6H), 1.42 (s, 3H), 1.49 (s, 6H), 1.55-1.73 (m, 3H), 1.91 (s, 6H), 2.74 (br. s., 1H), 3.80-4.04 (m, 1H), 4.34 (t, J 9.8 Hz, 0.6H), 4.68 (t, J 10.0 Hz, 0.4H), 5.25 (d, J 17.3 Hz, 0.4H), 5.49 (d, J 11.4 Hz, 1.6H), 6.02 (br. s., 1H), 6.88-7.00 (m, 2H), 7.06-7.16 (m, 1H), 7.25-7.45 (m, 2H), 7.60-7.83 (m, 1H), 8.33-8.50 (m, 1H), 9.70 (br. s., 1H). ESI-MS m/z calc. 566.2563, found 567.3 (M+1)⁺; Retention time: 2.44 minutes.

### Step 6: (11R)-6-(2,6-Dimethylphenyl)-11-isobutyl-9-oxa-2λ⁶-thia-3,5,12,19-tetrazatricyclo[12.3.1.14,8]nonadeca-1(18),4(19),5,7,14,16-hexaene 2,2-dioxide

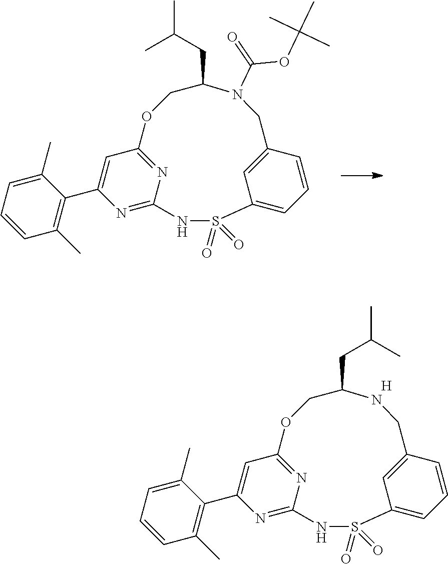

To a solution of tert-butyl (11R)-6-(2,6-dimethylphenyl)-11-(2-methylpropyl)-2,2-dioxo-9-oxa-2λ⁶-thia-3,5,12,19-tetraazatricyclo[12.3.1.14,8]nonadeca-1(17),4,6,8(19),14(18),15-hexaene-12-carboxylate (2.69 g, 4.7467 mmol) in DCM (30 mL) at 0° C. was added TFA (10 mL, 130.59 mmol). The mixture was stirred at rt for 2 h. The mixture was concentrated and co-evaporated with MeOH. The residue was purified by flash chromatography (80 g silica gel, DCM (1% NH₄OH)/MeOH 0-8%) to afford (11R)-6-(2,6-Dimethylphenyl)-11-isobutyl-9-oxa-2λ⁶-thia-3,5,12,19-tetrazatricyclo[12.3.1.14,8]nonadeca-1(18),4(19),5,7,14,16-hexaene 2,2-dioxide (2.002 g, 90%) as white solid. ¹H NMR (300 MHz, DMSO-d₆) δ 0.56 (d, J 6.5 Hz, 3H), 0.65 (d, J=6.5 Hz, 3H), 1.26-1.42 (m, 2H), 1.77 (dquin, J=13.5, 6.7 Hz, 1H), 2.00 (br. s., 6H), 2.23-2.33 (m, 1H), 3.69 (t, J 10.6 Hz, 1H), 3.87-4.00 (m, 1H), 4.05-4.20 (m, 1H), 5.23 (dd, J=10.4, 2.8 Hz, 1H), 6.27 (s, 1H), 7.04-7.15 (m, 2H), 7.18-7.28 (m, 1H), 7.42-7.54 (m, 2H), 7.61-7.71 (m, 1H), 8.56 (s, 1H). ESI-MS m/z calc. 466.2039, found 467.2 (M+1)⁺; Retention time: 1.89 minutes, LC method H.

### Step 7: (11R)-6-(2,6-Dimethylphenyl)-11-(2-methylpropyl)-12-[2-(oxan-4-yl)acetyl]-9-oxa-2λ⁶-thia-3,5,12,19-tetraazatricyclo[12.3.1.14,8]nonadeca-1(17),4,6,8(19),14(18),15-hexaene-2,2-dione (Compound 45)

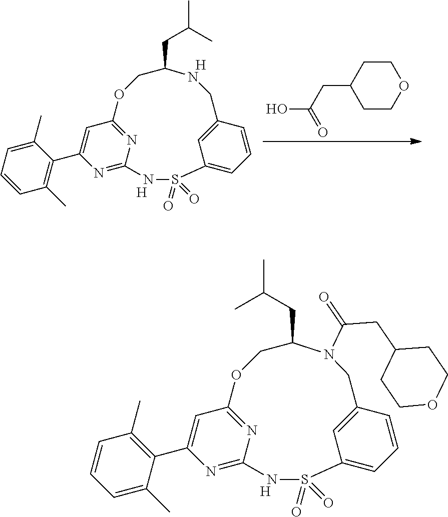

(11R)-6-(2,6-Dimethylphenyl)-11-isobutyl-9-oxa-2λ⁶-thia-3,5,12,19-tetrazatricyclo[12.3.1.14,8]nonadeca-1(18),4(19),5,7,14,16-hexaene 2,2-dioxide (40 mg, 0.08573 mmol), 2-tetrahydropyran-4-ylacetic acid (approximately 12.36 mg, 0.08573 mmol), HATU (approximately 48.90 mg, 0.1286 mmol), DIEA (approximately 44.32 mg, 59.73 μL, 0.3429 mmol), and DMF (1 mL) were stirred at room temperature for 2 h. The reaction mixture was filtered, and purified by reverse phase preparative chromatography using a C₁₈ column and a 15 min. gradient eluent of 30 to 99% acetonitrile in water containing 5 mM hydrochloric acid to give (11R)-6-(2,6-dimethylphenyl)-11-(2-methylpropyl)-12-[2-(oxan-4-yl)acetyl]-9-oxa-2λ⁶-thia-3,5,12,19-tetraazatricyclo[12.3.1.14,8]nonadeca-1(17),4,6,8(19),14(18),15-hexaene-2,2-dione (28.6 mg, 56%).ESI-MS m/z calc. 592.2719, found 593.0 (M+1)⁺; Retention time: 2.29 minutes; (LC method 1A).

### Example 33: Preparation of Compound 46

### Step 1: 3-[[4-(2,6-Dimethylphenyl)-6-[(2R)-2-[(4-methoxy-4-oxo-butyl)amino]-4-methyl-pentoxy]pyrimidin-2-yl]sulfamoyl]benzoic acid

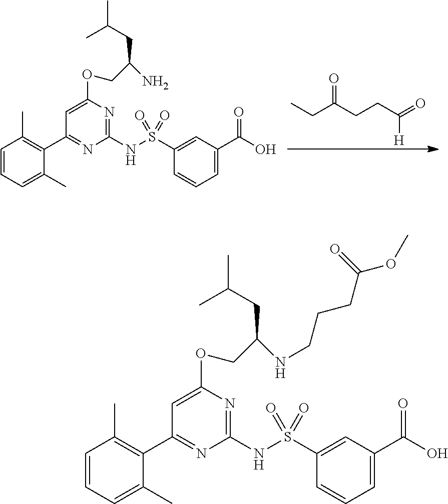

3-[[4-[(2R)-2-amino-4-methyl-pentoxy]-6-(2,6-dimethylphenyl)pyrimidin-2-yl]sulfamoyl]benzoic acid (hydrochloride salt) (293 mg, 0.5147 mmol), methyl 4-oxobutanoate (77.2 mg, 0.6649 mmol), and sodium triacetoxyborohydride (321 mg, 1.515 mmol) were combined in DCM (2 mL) and stirred at room temperature for 2 h. The reaction was partitioned between ethyl acetate and a 1M HCl solution. The organics were separated, washed with brine, dried over sodium sulfate and evaporated. The crude material was purified by reverse-phase HPLC utilizing a gradient of 1-99% acetonitrile in 5 mM aqueous HCl to yield 3-[[4-(2,6-dimethylphenyl)-6-[(2R)-2-[(4-methoxy-4-oxo-butyl)amino]-4-methyl-pentoxy]pyrimidin-2-yl]sulfamoyl]benzoic acid (106 mg, 34%). ESI-MS m/z calc. 598.2461, found 599.4 (M+1)⁺; Retention time: 0.47 minutes, LC method D.

### Step 2: 4-[(11R)-6-(2,6-Dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]butanoic acid

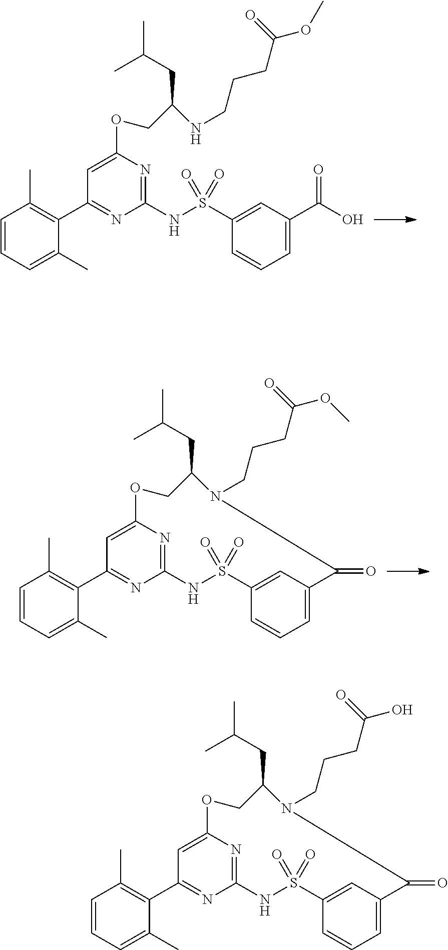 [SMILES: COC(=O)CCCN[C@@H](COc1cc(-c2c(C)cccc2C)nc(NS(=O)(=O)c2cccc(C(=O)O)c2)n1)CC(C)C ; COC(=O)CCCN1C(=O)c2cccc(c2)S(=O)(=O)Nc2nc(cc(-c3c(C)cccc3C)n2)OC[C@H]1CC(C)C ; Cc1cccc(C)c1-c1cc2nc(n1)NS(=O)(=O)c1cccc(c1)C(=O)N(CCCC(=O)O)[C@H](CC(C)C)CO2]

3-[[4-(2,6-Dimethylphenyl)-6-[(2R)-2-[(4-methoxy-4-oxo-butyl)amino]-4-methyl-pentoxy]pyrimidin-2-yl]sulfamoyl]benzoic acid (106 mg, 0.1770 mmol) was dissolved in DMF (4 mL). HATU (84.7 mg, 0.2228 mmol) was added, followed by triethylamine (100 μL, 0.7175 mmol) and the reaction was stirred for 30 min at room temperature. The reaction was partitioned between ethyl acetate and a 1M HCl solution. The organics were separated, washed with brine, dried over sodium sulfate and evaporated. The crude material was dissolved in a mixture of THE (2 mL): NaOH (2 mL of 1 M, 2.000 mmol) and the mixture was stirred at room temperature for 2 h. The reaction mixture was partitioned between ethyl acetate and a 1M HCl solution. The organics were separated, washed with brine, dried over sodium sulfate and evaporated to give 4-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]butanoic acid (100 mg, 100%) ESI-MS m/z calc. 566.2199, found 567.4 (M+1)⁺; Retention time: 0.59 minutes, LC method D.

### Step 3: 4-[(11R)-6-(2,6-Dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-12-yl]butan-1-ol (Compound 46)

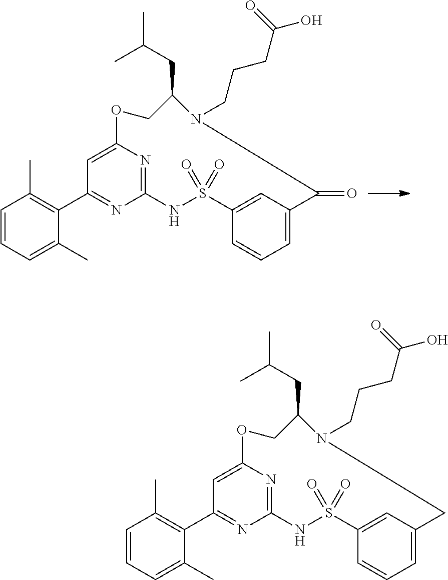 [SMILES: Cc1cccc(C)c1-c1cc2nc(n1)NS(=O)(=O)c1cccc(c1)C(=O)N(CCCC(=O)O)[C@H](CC(C)C)CO2 ; Cc1cccc(C)c1-c1cc2nc(n1)NS(=O)(=O)c1cccc(c1)CN(CCCC(=O)O)[C@H](CC(C)C)CO2]

4-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]butanoic acid (77 mg, 0.1359 mmol) was dissolved in borane tetrahydrofuran (1 mL of 1 M, 1.000 mmol) and stirred at room temperature for 1 h. The reaction was quenched with methanol and evaporated. The crude material was purified by reverse phase HPLC utilizing a gradient of 1-99% acetonitrile in 5 mM aqueous HCl to yield 4-[(11R)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-12-yl]butan-1-ol (hydrochloride salt) (15 mg, 19%) ESI-MS m/z calc. 538.26135, found 539.3 (M+1)⁺; Retention time: 1.18 minutes, LC method D.

### Example 34a: Preparation of Compound 47

Step 1: (11R)-6-(2,6-dimethylphenyl)-11-(2-methylpropyl)-12-[(pyridazin-4-yl)methyl]-9-oxa-2λ⁶-thia-3,5,12,19-tetraazatricyclo[12.3.1.14,8]nonadeca-1(17),4,6,8(19),14(18),15-hexaene-2,2-dione (Compound 47)

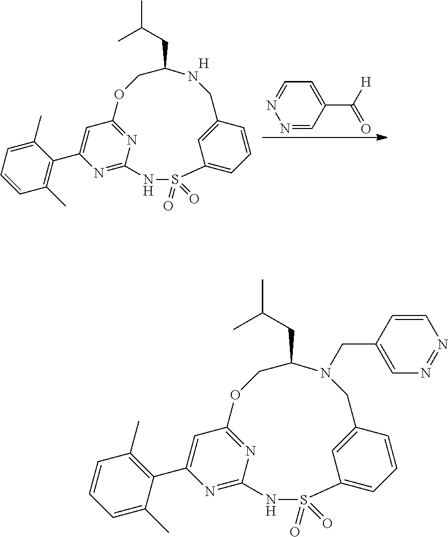

(11R)-6-(2,6-Dimethylphenyl)-11-isobutyl-9-oxa-2λ⁶-thia-3,5,12,19-tetrazatricyclo[12.3.1.14,8]nonadeca-1(18),4(19),5,7,14,16-hexaene 2,2-dioxide (20 mg, 0.04286 mmol), pyridazine-4-carbaldehyde (approximately 4.633 mg, 0.04286 mmol), HOAc (approximately 12.87 mg, 12.19 μL, 0.2143 mmol), in DCE (0.5 mL) were stirred at room temperature for 2 h. sodium triacetoxyborohydride (approximately 45.42 mg, 0.2143 mmol) was added to the mixture and the reactions was stirred at room temperature for 18 h. The reaction mixture was then diluted with methanol, filtered, and purified by reverse phase preparative chromatography using a C₁₈ column and a 15 min. gradient eluent of 30 to 99% acetonitrile in water containing 5 mM hydrochloric acid to give (11R)-6-(2,6-dimethylphenyl)-11-(2-methylpropyl)-12-[(pyridazin-4-yl)methyl]-9-oxa-2λ⁶-thia-3,5,12,19-tetraazatricyclo[12.3.1.14,8]nonadeca-1(17),4,6,8(19),14(18),15-hexaene-2,2-dione (10.4 mg, 41%). ESI-MS m/z calc. 558.24133, found 559.0 (M+1)⁺; Retention time: 1.58 minutes; LC method A. ¹H NMR (400 MHz, DMSO-d₆) δ 9.33 (s, 1H), 9.23 (s, 1H), 8.63 (s, 1H), 7.71 (d, J 24.7 Hz, 2H), 7.45 (d, J 34.5 Hz, 2H), 7.26 (s, 1H), 7.12 (s, 2H), 6.34 (s, 1H), 5.25 (s, 1H), 4.28 (s, 1H), 4.16 (d, J=15.7 Hz, 1H), 3.76 (s, 3H), 2.90 (s, 1H), 1.97 (s, 6H), 1.61 (s, 2H), 1.45 (s, 1H), 0.62 (s, 3H), 0.49 (s, 3H).

### Example 34b: Preparation of Compound 48

### Step 1: (11R)-6-(2,6-Dimethylphenyl)-11-(2,2-dimethylpropyl)-12-[(5-morpholino-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 48)

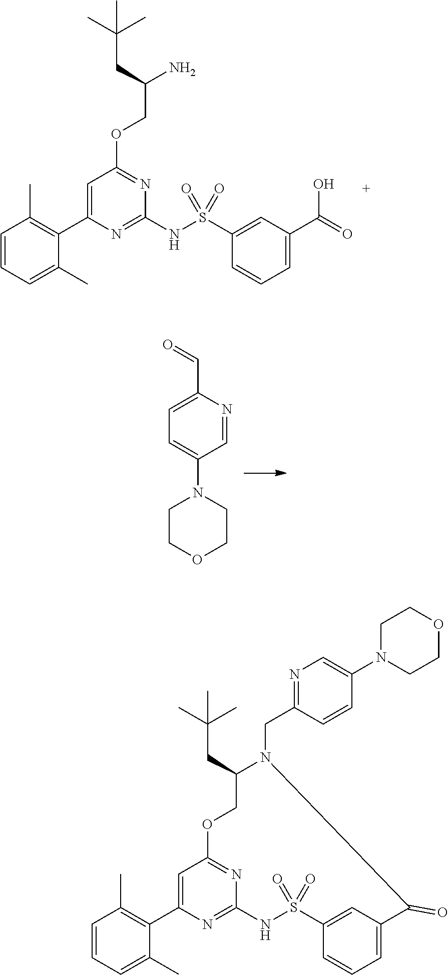 [SMILES: Cc1cccc(C)c1-c1cc(OC[C@H](N)CC(C)(C)C)nc(NS(=O)(=O)c2cccc(C(=O)O)c2)n1 ; O=Cc1ccc(N2CCOCC2)cn1 ; Cc1cccc(C)c1-c1cc2nc(n1)NS(=O)(=O)c1cccc(c1)C(=O)N(Cc1ccc(N3CCOCC3)cn1)[C@H](CC(C)(C)C)CO2]

3-[[4-[(2R)-2-amino-4,4-dimethyl-pentoxy]-6-(2,6-dimethylphenyl)pyrimidin-2-yl]sulfamoyl]benzoic acid (hydrochloride salt) (250 mg, 0.4553 mmol) was combined with 5-morpholinopyridine-2-carbaldehyde (105 mg, 0.5463 mmol) in DCM (750 μL) and stirred at room temperature for 15 minutes. Sodium triacetoxyborohydride (95 mg, 0.4482 mmol) was then added, followed by an additional sodium triacetoxyborohydride (240 mg, 1.132 mmol) 15 minutes later. The reaction was allowed to stir at room temperature for an additional 60 minutes, then was quenched with a small amount of 1M HCl, then was partially concentrated. After diluting with 1:1 DMSO/methanol and filtering, the reaction mixture was then purified by reverse phase HPLC (10-99ACN in water, HCl modifier, 15 min run) to give 3-[[4-[(2R)-4,4-dimethyl-2-[(5-morpholino-2-pyridyl)methylamino]pentoxy]-6-(2,6-dimethylphenyl)pyrimidin-2-yl]sulfamoyl]benzoic acid (hydrochloride salt) (245 mg, 74%) as a slightly yellow solid.

The product was dissolved in DMF (20 mL), and cooled to 0° C. in an ice bath. N-methylmorpholine (300 μL, 2.729 mmol) and CDMT (100 mg, 0.5696 mmol) were added, and the reaction was allowed to stir for an additional 20 minutes before the ice bath was removed and stirring was continued at room temperature for 2 hours. The reaction mixture was then concentrated by rotary evaporation, acidified with several drops of 1M HCl, diluted with 1:1 methanol/DMSO, filtered, and purified by reverse phase HPLC (10-99% ACN in water, HCl modifier, initially shallow gradient.) Fractions containing product were dried and combined to give (11R)-6-(2,6-dimethylphenyl)-11-(2,2-dimethylpropyl)-12-[(5-morpholino-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (75.2 mg, 22%). ¹H NMR (400 MHz, DMSO-d₆) δ 12.77 (s, 1H), 8.54 (s, 1H), 8.28 (d, J 2.8 Hz, 1H), 7.94 (d, J 6.6 Hz, 1H), 7.68 (m, 2H), 7.42-7.30 (m, 2H), 7.26 (t, J 7.6 Hz, 1H), 7.12 (d, J 7.7 Hz, 2H), 6.41 (s, 1H), 5.21 (dd, J 10.9, 4.3 Hz, 1H), 4.82 (d, J 15.3 Hz, 1H), 4.42 (d, J 15.3 Hz, 1H), 4.20 (t, J 11.2 Hz, 1H), 4.01 (d, J=10.3 Hz, 1H), 3.74 (dd, J=6.0, 3.6 Hz, 4H), 3.16 (t, J=4.8 Hz, 4H), 2.01 (m, 6H), 1.88-1.77 (m, 1H), 1.39 (d, J 15.0 Hz, 1H), 0.54 (s, 9H). ESI-MS m/z calc. 670.29376, found 671.6 (M+1)⁺; Retention time: 1.31 minutes (LC method A).

### Example 35: Preparation of Compound 49

### Step 1: 5-(Cyclopentoxy)pyridine-2-carbaldehyde

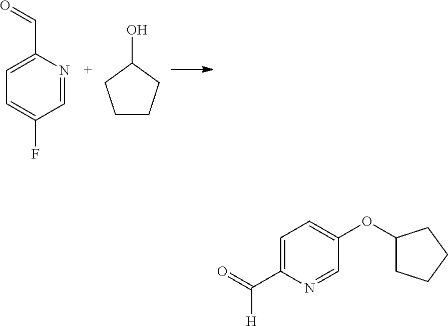

In a 4 mL vial, to a solution of cyclopentanol (100 mg, 1.161 mmol) in anhydrous DMF (2 mL) was added 5-fluoropyridine-2-carbaldehyde (120 mg, 0.9592 mmol), followed by addition of cesium carbonate (500 mg, 1.535 mmol). The vial was purged with nitrogen briefly and the capped heterogeneous mixture was stirred at 100° C. for 3 h. The reaction was allowed to cool to ambient temperature and the dark mixture was poured over cold 10% citric acid solution (15 mL) and extracted with ethyl acetate (3×15 mL). The combined organics were washed with water (15 mL), brine (15 mL), dried over sodium sulfate, filtered and concentrated under reduced pressure to give 5-(cyclopentoxy)pyridine-2-carbaldehyde (60 mg, 33%). ¹H NMR (400 MHz, Chloroform-d) δ 9.98 (d, J 0.8 Hz, 1H), 8.38 (d, J 2.8 Hz, 1H), 7.94 (d, J 8.7 Hz, 1H), 7.26 (dd, J=8.7, 2.8 Hz, 1H), 4.94-4.86 (m, 1H), 1.91-1.81 (m, 4H), 1.72-1.65 (m, 4H). ESI-MS m/z calc. 191.09464, found 192.2 (M+1)⁺; Retention time: 1.68 minutes (LC method A with a 1-50% gradient of MeCN).

### Step 2: 3-[[4-[(2R)-2-[[5-(Cyclopentoxy)-2-pyridyl]methylamino]-4,4-dimethyl-pentoxy]-6-(2,6-dimethylphenyl)pyrimidin-2-yl]sulfamoyl]benzoic acid

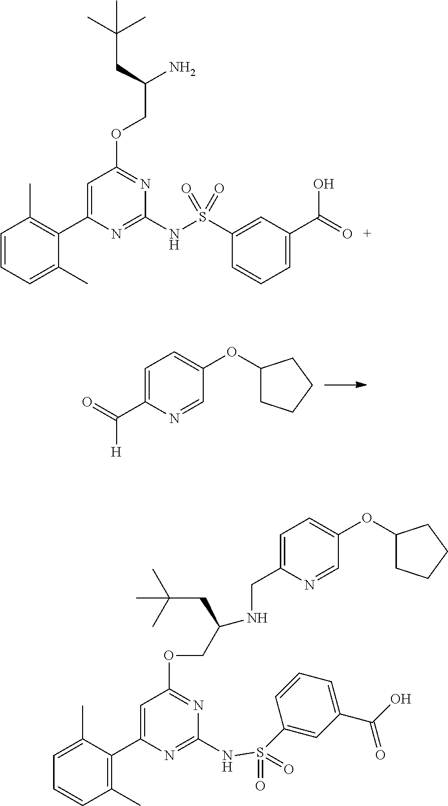

In a 4 mL vial, to a stirred heterogeneous mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (65 mg, 0.1184 mmol) in anhydrous dichloromethane (0.6 mL) were added 5-(cyclopentoxy)pyridine-2-carbaldehyde (23 mg, 0.1203 mmol), and glacial acetic acid (106.733 μL, 1.877 mmol), in that order. The vial was purged with nitrogen briefly and capped and stirred for 5 min, then sodium triacetoxyborohydride (130 mg, 0.6134 mmol) was added, followed by addition of DIEA (70 μL, 0.4019 mmol), and the capped vial was allowed to stir at ambient temperature for 90 min. Then methanol (0.3 mL) and water (0.2 mL) were added to the reaction and the volatiles were removed under reduced pressure and the residue was taken up in DMSO (1.5 mL), micro-filtered, and purified by reverse-phase HPLC (Cis column, 1-99% acetonitrile in water over 15 min, HCl as modifier) to furnish 3-[[4-[(2R)-2-[[5-(cyclopentoxy)-2-pyridyl]methylamino]-4,4-dimethyl-pentoxy]-6-(2,6-dimethylphenyl)pyrimidin-2-yl]sulfamoyl]benzoic acid (Dihydrochloride salt) (36 mg, 40%) as a white solid. ¹H NMR (400 MHz, DMSO-d₆) δ 13.43 (s, 1H), 9.18 (s, 2H), 8.45 (t, J 1.8 Hz, 1H), 8.26 (d, J 2.4 Hz, 1H), 8.13 (t, J 8.0 Hz, 2H), 7.68 (t, J 7.8 Hz, 1H), 7.53-7.42 (m, 2H), 7.26 (t, J 7.4 Hz, 1H), 7.13 (d, J 7.6 Hz, 2H), 6.32 (s, 1H), 4.92 (td, J 6.0, 3.2 Hz, 1H), 4.47-4.19 (m, 4H), 2.15-1.82 (m, 8H), 1.73-1.53 (m, 8H), 0.91 (s, 9H). ESI-MS m/z calc. 687.3091, found 688.3 (M+1)⁺; Retention time: 1.49 minutes (LC method A).

### Step 3: (11R)-12-[[5-(Cyclopentoxy)-2-pyridyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 49)

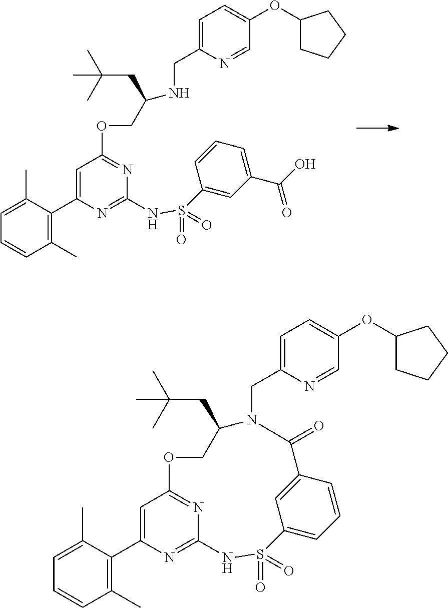 [SMILES: Cc1cccc(C)c1-c1cc(OC[C@@H](CC(C)(C)C)NCc2ccc(OC3CCCC3)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccc(OC3CCCC3)cn1)[C@H](CC(C)(C)C)CO2]

In a 4 mL vial, to a stirred solution of 3-[[4-[(2R)-2-[[5-(cyclopentoxy)-2-pyridyl]methylamino]-4,4-dimethyl-pentoxy]-6-(2,6-dimethylphenyl)pyrimidin-2-yl]sulfamoyl]benzoic acid (Dihydrochloride salt) (32 mg, 0.04206 mmol) in anhydrous DMF (1.5 mL) was added [dimethylamino(triazolo[4,5-b]pyridin-3-yloxy)methylene]-dimethyl-ammonium (Phosphorus Hexafluoride Ion) (35 mg, 0.09205 mmol) (HATU), followed by addition of DIEA (40 μL, 0.2296 mmol) at ambient temperature. The vial was briefly purged with nitrogen and the capped reaction was allowed to stir at ambient temperature for 1 h. The reaction was micro-filtered, and purified by preparative reverse-phase HPLC (1-99% acetonitrile in water over 15 min, HCl as modifier) to give (11R)-12-[[5-(cyclopentoxy)-2-pyridyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (14 mg, 49%) as a white solid. ESI-MS m/z calc. 669.29846, found 670.3 (M+1)⁺; Retention time: 1.8 minutes (LC method A).

### Example 36: Preparation of Compound 50

### Step 1: (11R)-12-[(5-bromopyridin-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione

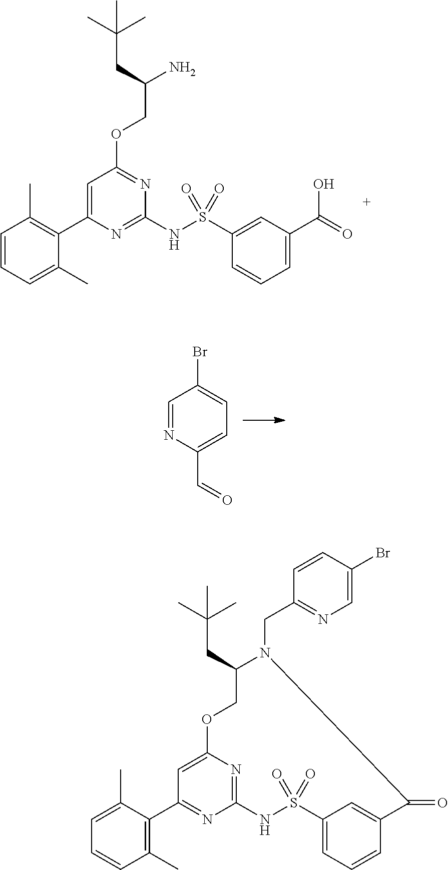

A flask was charged with 3-[[4-[(2R)-2-amino-4,4-dimethyl-pentoxy]-6-(2,6-dimethylphenyl)pyrimidin-2-yl]sulfamoyl]benzoic acid (hydrochloride salt) (2.5 g, 4.553 mmol) and 5-bromopyridine-2-carbaldehyde (1.02 g, 5.484 mmol) and DCM (10 mL) was added. The reaction mixture (slurry) was stirred at room temperature for 15 min then sodium triacetoxyborohydride (965 mg, 4.553 mmol) was added. After 20 minute and additional sodium triacetoxyborohydride (2.9 g, 13.68 mmol) was added and the reaction was continued to stir for an additional 40 minutes. The reaction mixture was partitioned between ethyl acetate and 1N HCl and saturated aqueous sodium chloride. The reaction mixture was extracted with ethyl acetate (3×) and the organic layer was washed with saturated sodium chloride solution. The organic layer was isolated, dried over anhydrous sodium sulfate, filtered, and evaporated to dryness. The crude material was slurried in 50% ethyl acetate/hexanes and filtered. The product was isolated as a white solid which contained some of the dialkylated side product This material 3-[[4-[(2R)-2-[(5-bromo-2-pyridyl)methylamino]-4,4-dimethyl-pentoxy]-6-(2,6-dimethylphenyl)pyrimidin-2-yl]sulfamoyl]benzoic acid (3.58 g, 115%) was used for the next step without further purification. ESI-MS m/z calc. 681.16205, found 682.37 (M+1)⁺; Retention time: 0.53 minutes (LC method D).

3-[[4-[(2R)-2-[(5-bromo-2-pyridyl)methylamino]-4,4-dimethyl-pentoxy]-6-(2,6-dimethylphenyl)pyrimidin-2-yl]sulfamoyl]benzoic acid (3.58 g, 115%) was dissolved in DMF (300 mL) and cooled to cooled in an ice water bath. 2-Chloro-4,6-dimethoxy-1,3,5-triazine (1.2 g, 6.835 mmol) was added to the reaction mixture followed by the addition of 4-methylmorpholine (3.22 g, 31.83 mmol) and the reaction was allowed to stir at 0° C. for 1 h. After 1 hour, the cooling batch was removed, and the reaction was warmed to room temperature and stirred at this temperature for 16 hours. The reaction was concentrated to a third of the volume then partitioned between ethyl acetate and 1N HCl solution. The organics were separated, dried over sodium sulfate, and evaporated to dryness. The crude material was purified by column chromatography on silica using 10-80% ethyl acetate/hexanes gradient to provide (11R)-12-[(5-bromopyridin-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (910 mg, 30%) as a white solid. ESI-MS m/z calc. 663.1515, found 664.32 (M+1)⁺; Retention time: 0.75 minutes (LC method A). ¹H NMR (400 MHz, DMSO-d₆) δ 13.07 (s, 1H), 8.68 (d, J 2.3 Hz, 1H), 8.60 (s, 1H), 8.05 (dd, J 8.4, 2.4 Hz, 1H), 7.95 (s, 1H), 7.68 (s, 2H), 7.47 (d, J 8.4 Hz, 1H), 7.26 (t, J 7.6 Hz, 1H), 7.13 (d, J 7.6 Hz, 2H), 6.43 (s, 1H), 5.30 (dd, J 10.9, 4.3 Hz, 1H), 4.81 (d, J 15.9 Hz, 1H), 4.57 (d, J 15.9 Hz, 1H), 4.26 (t, J=11.2 Hz, 1H), 4.08-3.97 (m, 1H), 2.02 (d, J=67.9 Hz, 6H), 1.79 (dd, J=15.3, 8.9 Hz, 1H), 1.44-1.34 (m, 1H), 0.55 (s, 9H).

### Step 2: (11R)-12-[[5-(Cyclohexen-1-yl)-2-pyridyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

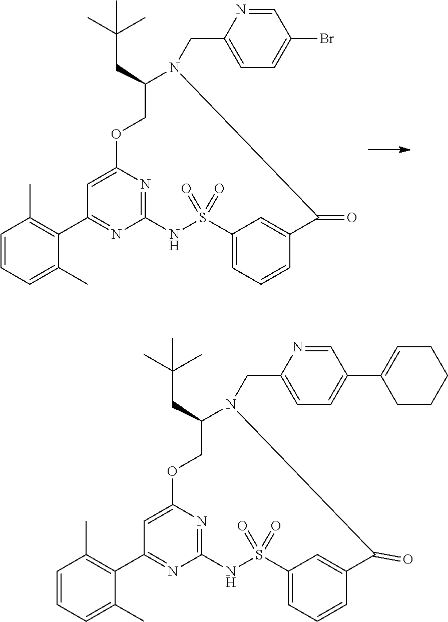 [SMILES: Cc1cccc(C)c1-c1cc2nc(n1)NS(=O)(=O)c1cccc(c1)C(=O)N(Cc1ccc(Br)cn1)[C@H](CC(C)(C)C)CO2 ; Cc1cccc(C)c1-c1cc2nc(n1)NS(=O)(=O)c1cccc(c1)C(=O)N(Cc1ccc(C3=CCCCC3)cn1)[C@H](CC(C)(C)C)CO2]

(11R)-12-[(5-bromo-2-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (15 mg, 0.02140 mmol) was combined with cyclohexen-1-ylboronic acid (approximately 5.391 mg, 0.04280 mmol), Pd(dppf)Cl₂ (approximately 0.7829 mg, 0.001070 mmol), and potassium carbonate (approximately 8.873 mg, 0.06420 mmol) in a nitrogen-purged vial with DMSO (0.2 mL) and DI water (0.05 mL). The reaction was heated to 110° C. for one hour. A second reaction was run: (11R)-12-[(5-bromo-2-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (15 mg, 0.02140 mmol) was combined with cyclohexen-1-ylboronic acid (approximately 5.391 mg, 0.04280 mmol), Pd(dppf)Cl₂ (approximately 0.7829 mg, 0.001070 mmol), and potassium carbonate (approximately 8.873 mg, 0.06420 mmol) in a nitrogen-purged vial with 1,4-dioxane (0.2 mL) and DI water (0.05 mL). The reaction was heated to 110° C. for one hour.

Both reactions were cooled to room temperature, combined, diluted with methanol and filtered, then purified by reverse phase HPLC (1-99% ACN in water, HCl modifier, 15 min run) to give (11R)-12-[[5-(cyclohexen-1-yl)-2-pyridyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) 17.9 mg, 60%). ESI-MS m/z calc. 665.3036, found 666.3 (M+1)⁺; Retention time: 1.79 minutes (LC method A).

### Step 3: (11R)-12-[(5-Cyclohexyl-2-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 50)

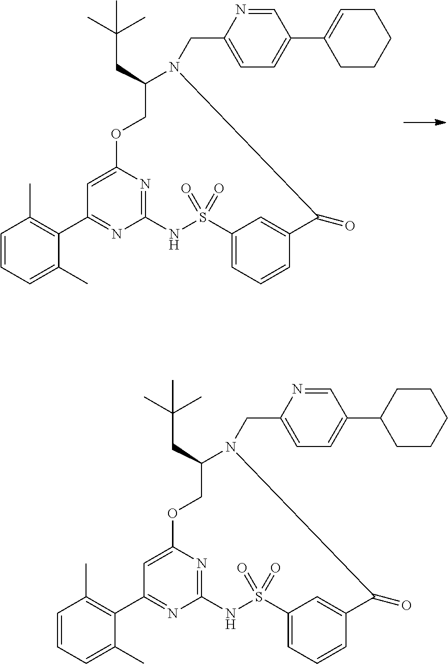 [SMILES: Cc1cccc(C)c1-c1cc2nc(n1)NS(=O)(=O)c1cccc(c1)C(=O)N(Cc1ccc(C3=CCCCC3)cn1)[C@H](CC(C)(C)C)CO2 ; Cc1cccc(C)c1-c1cc2nc(n1)NS(=O)(=O)c1cccc(c1)C(=O)N(Cc1ccc(C3CCCCC3)cn1)[C@H](CC(C)(C)C)CO2]

(11R)-12-[[5-(cyclohexen-1-yl)-2-pyridyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (15 mg, 0.02114 mmol) was combined with dihydroxypalladium (4.5 mg, 10% w/w, 0.003204 mmol) in a nitrogen-purged screwcap vial. methanol (1 mL) was added, and hydrogen gas was bubbled through the reaction mixture from a balloon for 30 minutes. Stirring was continued at room temperature with the balloon in place for an additional 2.5 hours. The reaction vessel was then flushed with nitrogen and the reaction mixture was filtered and purified by reverse phase HPLC (1-99% ACN in water, HCl modifier, 15 min run) to give as a white solid upon drying, (11R)-12-[(5-cyclohexyl-2-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (6.2 mg, 40%) ESI-MS m/z calc. 667.3192, found 668.7 (M+1)⁺; Retention time: 1.78 minutes (LC method A).

### Example 37: Preparation of Compound 51

### Step 1: ethyl 2-[(6-formyl-3-pyridyl)oxy]acetate

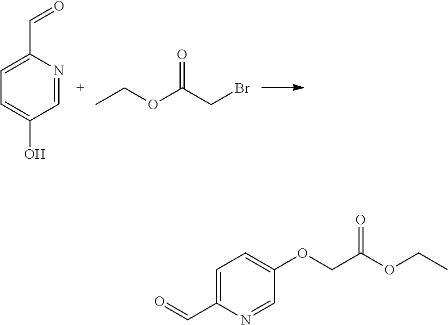

In a 20 mL vial, to a solution of 5-hydroxypyridine-2-carbaldehyde (300 mg, 2.437 mmol) in anhydrous DMF (4 mL) was added cesium carbonate (1.192 g, 3.658 mmol) and stirred at ambient temperature for 20 min. Then to the heterogeneous yellow mixture was added ethyl 2-bromoacetate (410 mg, 2.455 mmol) and stirred at 60° C. for 16 h. Upon cooling to ambient temperature, the reaction was quenched with water (20 mL) and extracted with ethyl acetate (3×20 mL). The combined organics were washed with water (2×15 mL) and brine (20 mL), dried over anhydrous sodium sulfate, filtered and concentrated under reduced pressure to furnish crude ethyl 2-[(6-formyl-3-pyridyl)oxy]acetate (269 mg, 53%) as pale-brown gum. It was used in the subsequent reaction without further purification. ¹H NMR (400 MHz, Chloroform-d) δ 10.00 (s, 1H), 8.47 (d, J 2.8 Hz, 1H), 7.97 (d, J 8.6 Hz, 1H), 7.29 (dd, J 8.7, 2.9 Hz, 1H), 4.76 (s, 2H), 4.30 (q, J 7.2 Hz, 2H), 1.31 (t, J 7.2 Hz, 3H). ESI-MS m z calc. 209.0688, found 210.1 (M+1)⁺; Retention time: 0.96 minutes (LC method A).

### Step 2: 3-[[4-(2,6-dimethylphenyl)-6-[(2R)-2-[[5-(2-ethoxy-2-oxo-ethoxy)-2-pyridyl]methylamino]-4,4-dimethyl-pentoxy]pyrimidin-2-yl]sulfamoyl]benzoic acid

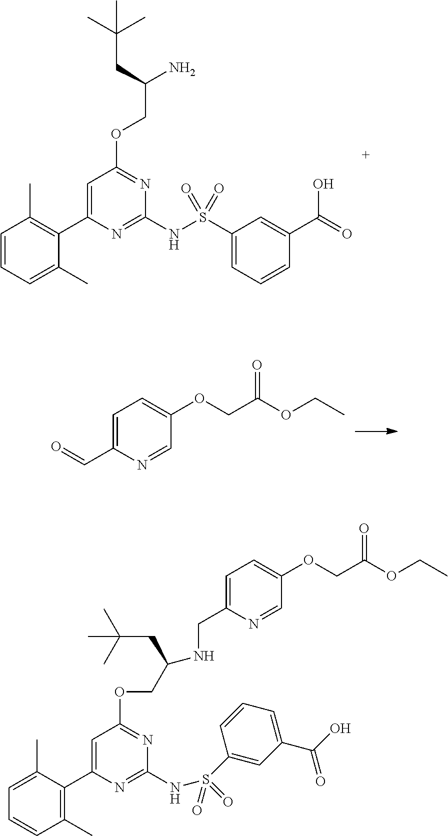 [SMILES: Cc1cccc(C)c1-c1cc(OC[C@H](N)CC(C)(C)C)nc(NS(=O)(=O)c2cccc(C(=O)O)c2)n1 ; CCOC(=O)COc1ccc(C=O)nc1 ; CCOC(=O)COc1ccc(CN[C@@H](COc2cc(-c3c(C)cccc3C)nc(NS(=O)(=O)c3cccc(C(=O)O)c3)n2)CC(C)(C)C)nc1]

In a 4 mL vial, to a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (200 mg, 0.3642 mmol) and ethyl 2-[(6-formyl-3-pyridyl)oxy]acetate (77 mg, 0.3681 mmol) in anhydrous dichloromethane (2 mL) were added glacial acetic acid (25 μL, 0.4396 mmol), sodium triacetoxyborohydride (400 mg, 1.887 mmol) and DIEA (200 μL, 1.148 mmol), in that order. The vial was purged with nitrogen briefly and capped and allowed to stir at ambient temperature for 1 h. Then methanol (0.8 mL) and water (0.5 mL) were added to the reaction and the volatiles were removed under reduced pressure and the residue was taken up in DMSO (1.5 mL), micro-filtered, and purified by (reverse-phase HPLC, 18 column, 1-99% acetonitrile in water over 15 min, HCl as modifier, single injection on big column). The desired fractions were dried in Genevac to furnish 3-[[4-(2,6-dimethylphenyl)-6-[(2R)-2-[[5-(2-ethoxy-2-oxo-ethoxy)-2-pyridyl]methylamino]-4,4-dimethyl-pentoxy]pyrimidin-2-yl]sulfamoyl]benzoic acid (hydrochloride salt) (115 mg, 43%) as a white solid. It was used in the subsequent reaction. ¹H NMR (400 MHz, DMSO-d₆) δ 13.36 (s, 1H), 9.23 (s, 2H), 8.46 (t, J 1.8 Hz, 1H), 8.38-8.29 (m, 1H), 8.14 (t, J 7.8 Hz, 2H), 7.68 (t, J 7.8 Hz, 1H), 7.56-7.43 (m, 2H), 7.26 (t, J 7.6 Hz, 1H), 7.13 (d, J 7.6 Hz, 2H), 6.34 (s, 1H), 4.87 (s, 2H), 4.48-4.23 (m, 4H), 4.16 (q, J 7.1 Hz, 2H), 3.43 (t, J 6.9 Hz, 1H), 2.02 (s, 6H), 1.67 (d, J 4.6 Hz, 2H), 1.21 (t, J 7.1 Hz, 3H), 0.91 (s, 9H). ESI-MS m/z calc. 705.28326, found 706.3 (M+1)⁺; Retention time: 1.29 minutes (LC method A).

### Step 3: Ethyl 2-[[6-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]methyl]-3-pyridyl]oxy]acetate

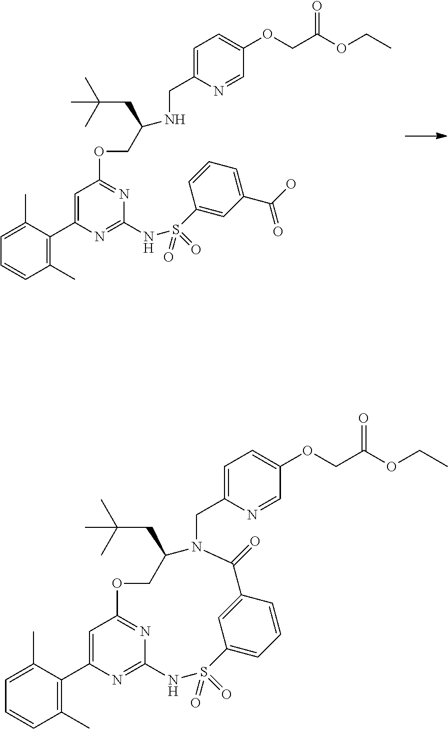 [SMILES: CCOC(=O)COc1ccc(CN[C@@H](COc2cc(-c3c(C)cccc3C)nc(NS(=O)(=O)c3cccc(C(=O)O)c3)n2)CC(C)(C)C)nc1 ; CCOC(=O)COc1ccc(CN2C(=O)c3cccc(c3)S(=O)(=O)Nc3nc(cc(-c4c(C)cccc4C)n3)OC[C@H]2CC(C)(C)C)nc1]

In a 25 mL flask, to a stirred solution of 3-[[4-(2,6-dimethylphenyl)-6-[(2R)-2-[[5-(2-ethoxy-2-oxo-ethoxy)-2-pyridyl]methylamino]-4,4-dimethyl-pentoxy]pyrimidin-2-yl]sulfamoyl]benzoic acid (hydrochloride salt) (108 mg, 0.1455 mmol) in anhydrous DMF (5 mL) was added [dimethylamino(triazolo[4,5-b]pyridin-3-yloxy)methylene]-dimethyl-ammonium (Phosphorus Hexafluoride Ion) (100 mg, 0.2630 mmol) (HATU), followed by addition of DIEA (130 μL, 0.7463 mmol) at ambient temperature. The flask was briefly purged with nitrogen and the capped reaction was allowed to stir at ambient temperature for 4 h. The volatiles were removed under reduced pressure and the residue was taken up in DMSO (1 mL) and methanol (0.2 mL) was added, micro-filtered, and purified by preparative reverse-phase HPLC (1-99% acetonitrile in water over 15 min, HCl as modifier) to give ethyl 2-[[6-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]methyl]-3-pyridyl]oxy]acetate (42 mg, 42%) as a white solid. It was used in the subsequent reaction. ¹H NMR (400 MHz, Chloroform-d) δ 8.69 (s, 1H), 8.29 (d, J 2.9 Hz, 1H), 8.10 (d, J 7.9 Hz, 1H), 7.84 (d, J 7.6 Hz, 1H), 7.64 (t, J 7.8 Hz, 1H), 7.54 (d, J 8.7 Hz, 1H), 7.29 (dd, J 8.6, 2.9 Hz, 1H), 7.20 (t, J 7.6 Hz, 1H), 7.04 (d, J 7.6 Hz, 2H), 6.21 (s, 1H), 5.28-5.12 (m, 2H), 4.68 (s, 2H), 4.40-4.24 (m, 3H), 4.23-4.11 (m, 2H), 1.98 (s, 6H), 1.82 (dd, J 15.3, 8.1 Hz, 1H), 1.52 (d, J 15.1 Hz, 1H), 1.31 (t, J 7.1 Hz, 3H), 0.60 (s, 9H). ESI-MS m/z calc. 687.27264, found 688.3 (M+1)⁺; Retention time: 1.61 minutes (LC method A).

### Step 4: (11R)-6-(2,6-Dimethylphenyl)-11-(2,2-dimethylpropyl)-12-[[5-(2-hydroxy-2-methyl-propoxy)-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 51)

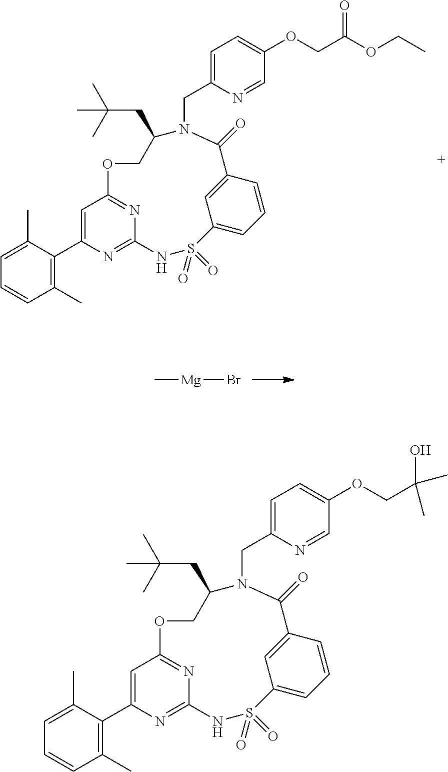

To a stirred solution of ethyl 2-[[6-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]methyl]-3-pyridyl]oxy]acetate (20 mg, 0.02908 mmol) in anhydrous tetrahydrofuran (0.5 mL), a solution of methyl magnesium bromide (60 μL of 3.0 M, 0.1800 mmol) (3.0 M in diethyl ether) was added dropwise at 0-5° C. (ice-water bath) under nitrogen. The reaction mixture was stirred for 30 min, then quenched by slow addition of acetic acid (100 μL), and purified by reverse-phase preparative HPLC (10-99% acetonitrile in water over 15 min, and 5 mM HCl as a modifier) to give (11R)-6-(2,6-dimethylphenyl)-11-(2,2-dimethylpropyl)-12-[[5-(2-hydroxy-2-methyl-propoxy)-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (6 mg, 30%) as white solid. ¹H NMR (400 MHz, DMSO-d₆) δ 8.58 (s, 1H), 8.34 (d, J 2.7 Hz, 1H), 7.95 (dd, J 5.4, 2.8 Hz, 1H), 7.69 (d, J 6.4 Hz, 2H), 7.59-7.43 (m, 2H), 7.26 (t, J 7.6 Hz, 1H), 7.13 (d, J 7.6 Hz, 2H), 6.43 (s, 1H), 5.26 (dd, J 10.8, 4.5 Hz, 1H), 4.89-4.78 (m, 1H), 4.54 (dd, J 15.6, 7.7 Hz, 1H), 4.26 (t, J 11.2 Hz, 1H), 4.07-3.96 (m, 1H), 3.84 (s, 2H), 2.01 (s, 6H), 1.82 (dd, J 15.2, 8.9 Hz, 1H), 1.40 (d, J 15.0 Hz, 1H), 1.21 (s, 6H), 0.54 (s, 9H). (missing NH and OH peaks) ESI-MS m/z calc. 673.2934, found 674.4 (M+1)⁺; Retention time: 1.57 minutes (LC method A).

### Example 38: Preparation of Compound 52

### Step 1: 5-(3,6-Dihydro-2H-pyran-4-yl)pyridine-2-carbaldehyde

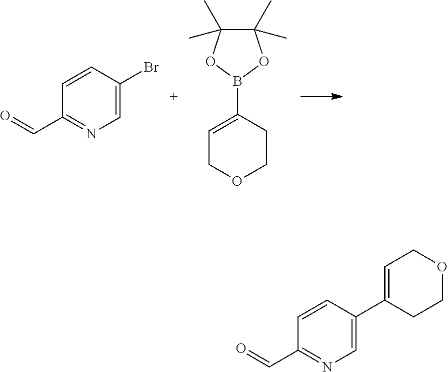

To a sealed tube was added 5-bromopyridine-2-carbaldehyde (2.5 g, 13.440 mmol), 2-(3,6-dihydro-2H-pyran-4-yl)-4,4,5,5-tetramethyl-1,3,2-dioxaborolane (3 g, 14.416 mmol) in degassed 1,4-dioxane (50 mL). A degassed solution of potassium carbonate (5.6 g, 40.519 mmol) in water (12.5 mL) was added followed by 1,1′-bis(diphenylphosphino)ferrocene palladium(II) chloride, complex with dichloromethane (220 mg, 0.2694 mmol). The tube was sealed and the reaction mixture was stirred at 80° C. for 18 hours and then cooled to room temperature. The mixture was filtered on Celite, the pad was rinsed with EtOAc (100 mL) and the filtrate was concentrated in vacuo. The mixture was triturated from MTBE (100 mL) and filtered. Afforded 5-(3,6-dihydro-2H-pyran-4-yl)pyridine-2-carbaldehyde (1.05 g, 39%) as a brown solid. ¹H NMR (400 MHz, CDCl₃) δ 10.08 (s, 1H), 8.84 (d, J 2.0 Hz, 1H), 7.96 (d, J 8.1 Hz, 1H), 7.84 (dd, J 8.1, 2.0 Hz, 1H), 6.44-6.33 (m, 1H), 4.39 (q, J 2.9 Hz, 2H), 3.99 (t, J 5.4 Hz, 2H), 2.71-2.49 (m, 2H). ESI-MS m/z calc. 189.079, found 190.2 (M+1)⁺; Retention time: 1.4 minutes. The filtrate was concentrated under reduced pressure and the resulting semi-solid was triturated from MTBE (15 mL) and filtered. Afforded 5-(3,6-dihydro-2H-pyran-4-yl)pyridine-2-carbaldehyde (0.6 g, 22%) as a brown solid. ¹H NMR (400 MHz, CDCl₃) δ 10.08 (s, 1H), 8.84 (d, J 2.0 Hz, 1H), 7.96 (d, J 8.1 Hz, 1H), 7.84 (dd, J 8.1, 2.0 Hz, 1H), 6.44-6.33 (m, 1H), 4.39 (q, J 2.9 Hz, 2H), 3.99 (t, J 5.4 Hz, 2H), 2.71-2.49 (m, 2H).LC method X.

### Step 2: (5-Tetrahydropyran-4-yl-2-pyridyl)methanol

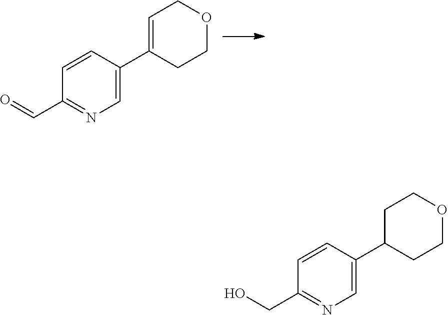 [SMILES: O=Cc1ccc(C2=CCOCC2)cn1 ; OCc1ccc(C2CCOCC2)cn1]

Palladium 5% on carbon (200 mg, 0.0940 mmol) was added into a 250 mL-flask and it was purged with nitrogen for 2 minutes. A solution of 5-(3,6-dihydro-2H-pyran-4-yl)pyridine-2-carbaldehyde (1.65 g, 8.5460 mmol) in methanol (30 mL) was then added into the flask. Hydrogen was bubbled into the suspension for 2 minutes and then the reaction mixture was stirred under hydrogen atmosphere for 18 hours. Nitrogen was then bubbled into the mixture for 10 minutes. The reaction mixture was filtered on Celite, the pad was rinsed with EtOAc (100 mL) and the filtrate was concentrated in vacuo. Afforded crude (5-tetrahydropyran-4-yl-2-pyridyl)methanol (1 g, 58%) as a light yellow solid. ¹H NMR (400 MHz, CDCl₃) δ 8.46 (d, J 1.7 Hz, 1H), 7.55 (dd, J 8.1, 2.2 Hz, 1H), 7.21 (d, J 8.1 Hz, 1H), 4.75 (d, J 3.2 Hz, 2H), 4.17-4.07 (m, 2H), 3.65-3.51 (m, 3H), 2.89-2.76 (m, 1H), 1.93-1.73 (m, 4H). ESI-MS m/z calc. 193.1103, found 194.2 (M+1)⁺; Retention time: 0.25 minutes (LC method X).

### Step 3: 5-Tetrahydropyran-4-ylpyridine-2-carbaldehyde

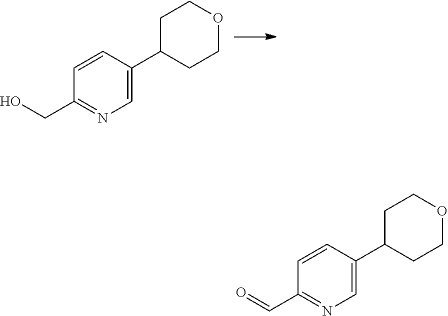 [SMILES: OCc1ccc(C2CCOCC2)cn1 ; O=Cc1ccc(C2CCOCC2)cn1]

To a solution of (5-tetrahydropyran-4-yl-2-pyridyl)methanol (1 g, 5.0714 mmol) in water saturated DCM (25 mL) was added Dess-Martin periodinane (2.39 g, 5.6349 mmol) and the reaction was then stirred for 1 hour at room temperature. A mixture of aqueous saturated solutions of Na₂S₂O₃ (20 mL), saturated aqueous sodium bicarbonate (20 mL), water (10 mL) and 1N aqueous NaOH (6 mL, to reach pH=7) was added and the reaction mixture was stirred for 10 minutes. The phases were separated, and the aqueous layer was extracted with DCM (3×50 mL). The combined organic layers were dried over sodium sulfate, filtered and concentrated in vacuo. The crude product was purified by reverse phase chromatography using a 100 g C₁₈ cartridge, eluting with a gradient of MeCN in water (5% to 100%). Volatiles were removed from the fractions containing the product. The resulting aqueous phase was extracted with DCM (3×30 mL). The combined organic layers were washed with brine (30 mL), dried over sodium sulfate, filtered and concentrated in vacuo. Afforded 5-tetrahydropyran-4-ylpyridine-2-carbaldehyde (730 mg, 74%) as a white solid. ¹H NMR (400 MHz, CDCl₃) δ 10.08 (d, J 0.7 Hz, 1H), 8.68 (d, J 2.0 Hz, 1H), 7.95 (d, J 8.1 Hz, 1H), 7.73 (dd, J 8.1, 2.2 Hz, 1H), 4.19-4.08 (m, 2H), 3.57 (td, J 11.5, 2.7 Hz, 2H), 3.02-2.82 (m, 1H), 1.94-1.79 (m, 4H). ESI-MS m/z calc. 191.0946, found 192.1 (M+1)⁺; Retention time: 2.24 minutes (LC method X).

### Step 4: 3-[[4-(2,6-Dimethylphenyl)-6-[(2R)-4,4-dimethyl-2-[(5-tetrahydropyran-4-yl-2-pyridyl)methylamino]pentoxy]pyrimidin-2-yl]sulfamoyl]benzoic acid

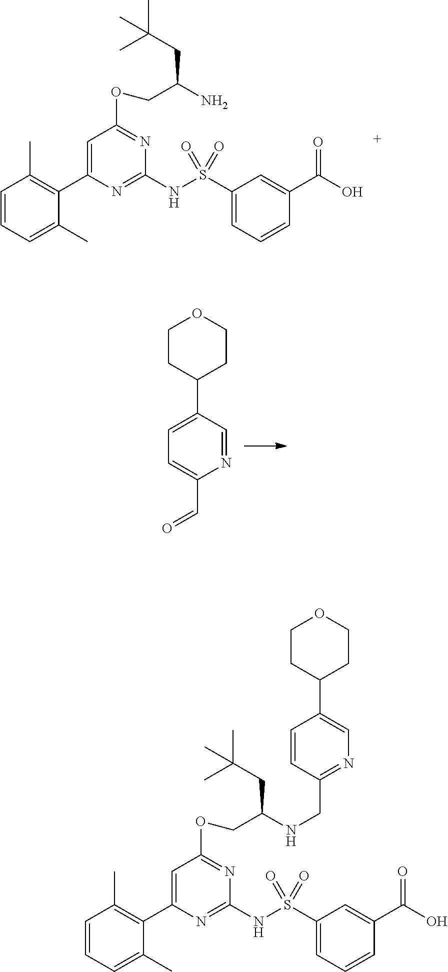 [SMILES: Cc1cccc(C)c1-c1cc(OC[C@H](N)CC(C)(C)C)nc(NS(=O)(=O)c2cccc(C(=O)O)c2)n1 ; O=Cc1ccc(C2CCOCC2)cn1 ; Cc1cccc(C)c1-c1cc(OC[C@@H](CC(C)(C)C)NCc2ccc(C3CCOCC3)cn2)nc(NS(=O)(=O)c2cccc(C(=O)O)c2)n1]

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (90 mg, 0.1542 mmol) in anhydrous dichloromethane (0.5 mL) was added 5-tetrahydropyran-4-ylpyridine-2-carbaldehyde (67 mg, 0.1542 mmol) followed after 10 min by sodium triacetoxyborohydride (98 mg, 0.4624 mmol). The reaction was stirred at ambient temperature for 30 min under nitrogen and 1 mL of 1 N aqueous HCl was added at 0° C. The mixture was stirred at room temperature for 1 h and concentrated under reduced pressure and co-evaporated with toluene (10 mL). Afforded 3-[[4-(2,6-dimethylphenyl)-6-[(2R)-4,4-dimethyl-2-[(5-tetrahydropyran-4-yl-2-pyridyl)methylamino]pentoxy]pyrimidin-2-yl]sulfamoyl]benzoic acid (106 mg, 100%) that was used as is without further analysis.

### Step 5: (11R)-6-(2,6-Dimethylphenyl)-11-(2,2-dimethylpropyl)-2,2-dioxo-12-[(5-tetrahydropyran-4-yl-2-pyridyl)methyl]-9-oxa-2λ⁶-thia-3,5,12,19-tetrazatricyclo[12.3.1.14,8]nonadeca-1(18),4(19),5,7,14,16-hexaen-13-one (Compound 52)

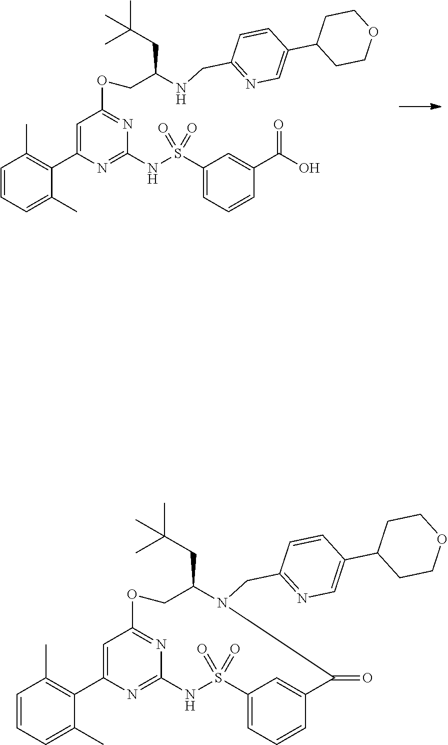 [SMILES: Cc1cccc(C)c1-c1cc(OC[C@@H](CC(C)(C)C)NCc2ccc(C3CCOCC3)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccc(C3CCOCC3)cn1)[C@H](CC(C)(C)C)CO2]

To a 0° C. solution of N-methylmorpholine (132.48 mg, 144 μL, 1.3098 mmol) in DMF (17 mL) was added 2-chloro-4,6-dimethoxy-1,3,5-triazine (42 mg, 0.2392 mmol) followed by 3-[[4-(2,6-dimethylphenyl)-6-[(2R)-4,4-dimethyl-2-[(5-tetrahydropyran-4-yl-2-pyridyl)methylamino]pentoxy]pyrimidin-2-yl]sulfamoyl]benzoic acid (106 mg, 0.0946 mmol). After 5 min the reaction was stirred at room temperature for 72 h. The reaction mixture was concentrated under reduced pressure at 50° C. and the crude was directly loaded on a 30 g C₁₈ cartridge, eluting with a gradient of MeCN in acidic water (0.1% v/v of formic acid in water) of 50 to 100%. The fractions containing the product were concentrated to dryness under reduced pressure, co-evaporated twice with water (2 mL) and lyophilized. Afforded (11R)-6-(2,6-dimethylphenyl)-11-(2,2-dimethylpropyl)-2,2-dioxo-12-[(5-tetrahydropyran-4-yl-2-pyridyl)methyl]-9-oxa-2λ⁶-thia-3,5,12,19-tetrazatricyclo[12.3.1.14,8]nonadeca-1(18),4(19),5,7,14,16-hexaen-13-one (7 mg, 11%) as a white solid. ¹H NMR (400 MHz, DMSO-d₆) δ 13.46-12.58 (m, 1H), 8.71-8.57 (m, 1H), 8.49 (d, J=1.7 Hz, 1H), 8.04-7.89 (m, 1H), 7.81-7.59 (m, 3H), 7.49-7.37 (m, 1H), 7.33-7.21 (m, 1H), 7.19-7.07 (m, 2H), 6.53-6.36 (m, 1H), 5.33 (br dd, J 10.3, 3.9 Hz, 1H), 4.83 (d, J 15.7 Hz, 1H), 4.55 (br d, J 15.4 Hz, 1H), 4.24 (t, J 11.2 Hz, 1H), 4.10-4.00 (m, 1H), 4.00-3.90 (m, 2H), 3.47-3.40 (m, 2H), 2.92-2.79 (m, 1H), 2.23-1.88 (m, 6H), 1.80 (br dd, J 15.2, 9.0 Hz, 1H), 1.75-1.67 (m, 4H), 1.40 (br d, J 14.9 Hz, 1H), 0.55 (s, 9H). ESI-MS m/z calc. 669.2985, found 670.3 (M+1)⁺; Retention time: 4.07 minutes (LC method Y).

### Example 39: Preparation of Compound 53

### Step 1: (11R)-12-[(3-Bromo-2-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

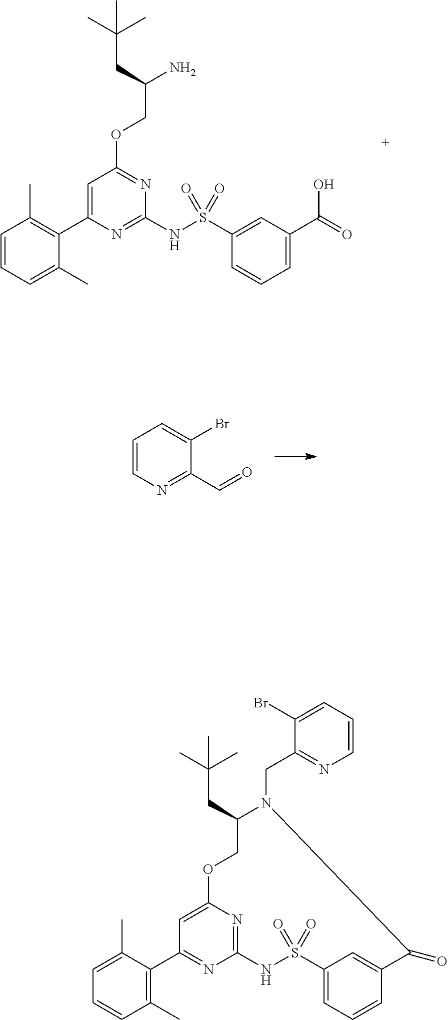 [SMILES: Cc1cccc(C)c1-c1cc(OC[C@H](N)CC(C)(C)C)nc(NS(=O)(=O)c2cccc(C(=O)O)c2)n1 ; O=Cc1ncccc1Br ; Cc1cccc(C)c1-c1cc2nc(n1)NS(=O)(=O)c1cccc(c1)C(=O)N(Cc1ncccc1Br)[C@H](CC(C)(C)C)CO2]

In a reaction vial, 3-[[4-[(2R)-2-amino-4,4-dimethyl-pentoxy]-6-(2,6-dimethylphenyl)pyrimidin-2-yl]sulfamoyl]benzoic acid (hydrochloride salt) (600 mg, 1.093 mmol) was mixed with 3-bromopyridine-2-carbaldehyde (407 mg, 2.188 mmol) in methylene chloride (2.88 mL). The reaction mixture was stirred at rt for 15 min then sodium triacetoxyborohydride (695 mg, 3.279 mmol) was added. The reaction was stirred at rt for 3 h. then partitioned between ethyl acetate and 1N HCl. The reaction mixture was extracted with ethyl acetate (3×) and the organic layer was washed with saturated NaCl solution. The organic layer was isolated, dried over anhydrous sodium sulfate, filtered, and evaporated to dryness. The crude material was slurried in 50% ethyl acetate/hexanes and filtered. The intermediate was isolated as a light-yellow solid which contained some of the dialkylated side product. This material was used for the next step without further purification.

In a reaction vial, 3-[[4-[(2R)-2-[(3-bromo-2-pyridyl)methylamino]-4,4-dimethyl-pentoxy]-6-(2,6-dimethylphenyl)pyrimidin-2-yl]sulfamoyl]benzoic acid (hydrochloride salt) (568.9 mg, 72%) was dissolved in DMF (29.4 mL) along with 4-methylmorpholine (221 mg, 2.185 mmol) and cooled to 0° C. To the reaction, 2-chloro-4,6-dimethoxy-1,3,5-triazine (192 mg, 1.094 mmol) was added and the reaction was allowed to stir at 0° C. for 1 h. then additional 4-methylmorpholine (111 mg, 1.097 mmol) was added. The reaction was allowed to warm to rt and stirred at that temperature for 4 h. The reaction was concentrated to a third of the volume then partitioned between ethyl acetate and 1N HCl solution. The organics were separated, dried over sodium sulfate, and evaporated to dryness. The crude material was purified by column chromatography on silica using 10-80% ethyl acetate/hexanes gradient. The product was isolated as a white solid. (11R)-12-[(3-bromo-2-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (141 mg, 19%) ¹H NMR (400 MHz, DMSO-d₆) δ 13.03 (s, 1H), 8.77 (s, 1H), 8.55 (dd, J 4.7, 1.5 Hz, 1H), 8.09 (dd, J 8.1, 1.5 Hz, 1H), 7.95 (s, 1H), 7.67 (s, 2H), 7.31-7.22 (m, 2H), 7.12 (d, J 7.6 Hz, 2H), 6.40 (s, 1H), 5.47-5.40 (m, 1H), 5.09 (d, J 16.6 Hz, 1H), 4.55 (d, J 16.7 Hz, 1H), 4.14 (t, J 11.1 Hz, 1H), 4.09-3.99 (m, 1H), 3.97 (s, 2H), 3.32 (s, 2H), 2.09 (s, 3H), 1.95 (s, 4H), 1.77 (dd, J 15.2, 8.6 Hz, 1H), 1.49-1.37 (m, 1H), 1.30-1.22 (m, 1H), 0.85 (dt, J=10.6, 6.7 Hz, 1H), 0.59 (s, 9H). ESI-MS m z calc. 663.1515, found 664.2 (M+1)⁺; Retention time: 2.0 minutes (LC method A).

### Step 2: (11R)-6-(2,6-Dimethylphenyl)-11-(2,2-dimethylpropyl)-2,2-dioxo-12-[[3-(1H-pyrazol-4-yl)-2-pyridyl]methyl]-9-oxa-2λ⁶-thia-3,5,12,19-tetrazatricyclo[12.3.1.14,8]nonadeca-1(18),4(19),5,7,14,16-hexaen-13-one (Compound 53)

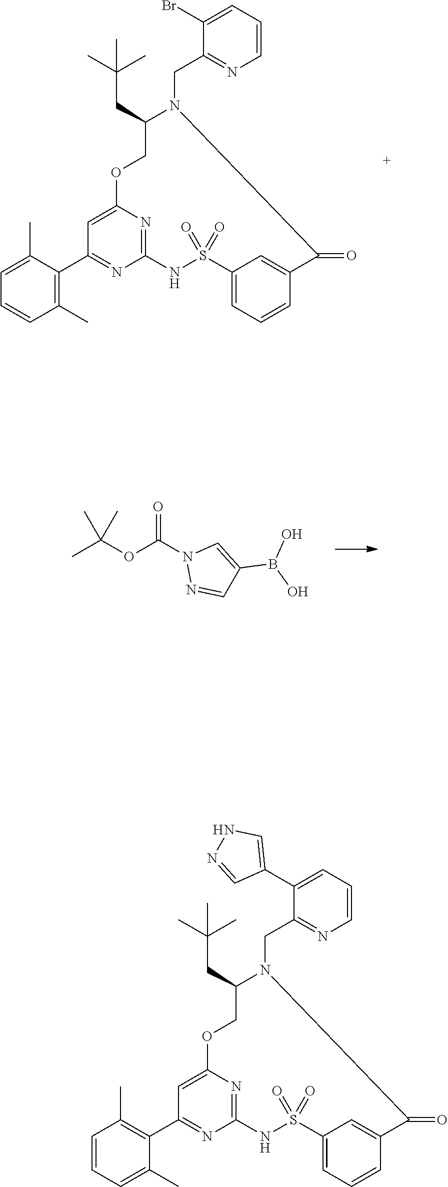 [SMILES: Cc1cccc(C)c1-c1cc2nc(n1)NS(=O)(=O)c1cccc(c1)C(=O)N(Cc1ncccc1Br)[C@H](CC(C)(C)C)CO2 ; CC(C)(C)OC(=O)n1cc(B(O)O)cn1 ; Cc1cccc(C)c1-c1cc2nc(n1)NS(=O)(=O)c1cccc(c1)C(=O)N(Cc1ncccc1-c1cn[nH]c1)[C@H](CC(C)(C)C)CO2]

In a microwave reaction vial, (11R)-12-[(3-bromo-2-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (30 mg, 0.04514 mmol) was mixed with the specified boronic acid/ester (1-tert-butoxycarbonylpyrazol-4-yl)boronic acid (approximately 10.77 mg, 0.05078 mmol) and potassium carbonate (70.5 μL of 2 M, 0.1410 mmol) in DMSO (600 μL). The reaction was flushed with nitrogen then Pd(dppf)Cl₂ (3 mg, 0.003674 mmol) was added. The reaction was purged again with nitrogen and heated at 120° C. for 45 min in the microwave. The reaction mixture was diluted with ethyl acetate and washed with 1N HCl followed by saturated NaCl solution. The organic layer was isolated, dried over anhydrous sodium sulfate, filtered, and evaporated to dryness. The crude material was purified by preparative HPLC to give (11R)-6-(2,6-dimethylphenyl)-11-(2,2-dimethylpropyl)-2,2-dioxo-12-[[3-(1H-pyrazol-4-yl)-2-pyridyl]methyl]-9-oxa-2λ⁶-thia-3,5,12,19-tetrazatricyclo[12.3.1.14,8]nonadeca-1(18),4(19),5,7,14,16-hexaen-13-one (7 mg, 24%). ESI-MS m/z calc. 651.26276, found 652.2 (M+1)⁺; Retention time: 1.49 minutes; LC method A.

### Example 40: Preparation of Compound 54

### Step 1: 3-(Methoxymethoxy)pyridine-2-carbaldehyde

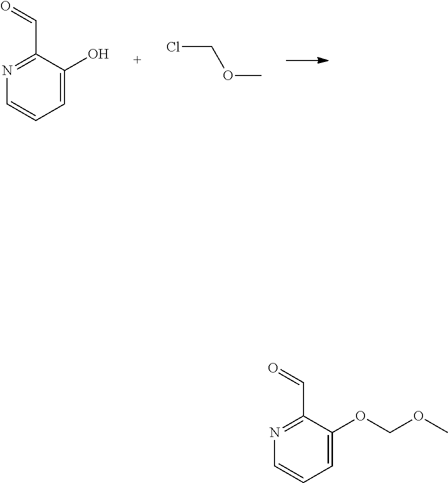

3-hydroxypyridine-2-carbaldehyde (200 mg, 1.625 mmol) was dissolved in DCM (6 mL) and cooled to 0° C. DIPEA (560 μL, 3.215 mmol) was added, followed by dropwise addition of chloro(methoxy)methane (185 μL, 2.436 mmol). The ice bath was allowed to slowly melt and stirring was continued at room temperature for 60 hours. The reaction mixture was then poured into 30 mL DI water and extracted 3× with 20 mL dichloromethane. The combined organics were washed with brine, dried over sodium sulfate and concentrated to give a yellow oil. The resulting material was used in the next step without further purification. 3-(methoxymethoxy)pyridine-2-carbaldehyde (265 mg, 98%) ¹H NMR (400 MHz, Chloroform-d) δ 10.38 (s, 1H), 8.46 (dd, J 4.5, 1.3 Hz, 1H), 7.67 (dd, J 8.5, 1.3 Hz, 1H), 7.46 (dd, J 8.6, 4.4 Hz, 1H), 5.35 (s, 2H), 3.53 (s, 3H). ESI-MS m/z calc. 167.05824, found 168.1 (M+1)⁺; Retention time: 0.45 minutes (LC method A).

### Step 2: (11R)-6-(2,6-Dimethylphenyl)-11-(2,2-dimethylpropyl)-12-[(3-hydroxy-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 54)

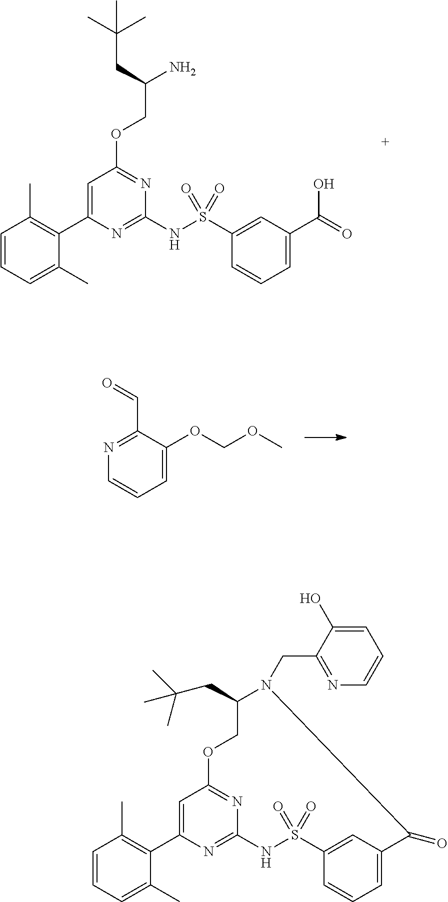 [SMILES: Cc1cccc(C)c1-c1cc(OC[C@H](N)CC(C)(C)C)nc(NS(=O)(=O)c2cccc(C(=O)O)c2)n1 ; COCOc1cccnc1C=O ; Cc1cccc(C)c1-c1cc2nc(n1)NS(=O)(=O)c1cccc(c1)C(=O)N(Cc1ncccc1O)[C@H](CC(C)(C)C)CO2]

3-[[4-[(2R)-2-amino-4,4-dimethyl-pentoxy]-6-(2,6-dimethylphenyl)pyrimidin-2-yl]sulfamoyl]benzoic acid (hydrochloride salt) (100 mg, 0.1821 mmol) was combined with 3-(methoxymethoxy)pyridine-2-carbaldehyde (approximately 36.52 mg, 0.2185 mmol) in DCM (364.2 μL) and stirred at room temperature for 15 minutes. Sodium triacetoxyborohydride (approximately 38.59 mg, 0.1821 mmol) (1 equivalent) was added then added, followed by additional sodium triacetoxyborohydride (approximately 115.8 mg, 0.5463 mmol) (3 equivalents) 15 minutes later. The reaction mixture was then stirred at room temperature for 2 hours. After this time the reaction mixture was quenched into 1M HCl and extracted 4× with ethyl acetate. The combined organics were washed with brine, dried over sodium sulfate and concentrated. The resulting crude material was purified by reverse phase HPLC (1-70% ACN in water, HCl modifier, 15 min run) to give the corresponding reductive amination product.

The product from step 1 was combined with N-methylmorpholine (approximately 110.6 mg, 120.2 μL, 1.093 mmol) in DMF (4 mL) and cooled to 0° C. Solid CDMT (approximately 47.97 mg, 0.2732 mmol) was added and the reaction was stirred at 0° C. for 1 hour then room temperature for an additional 4 hours. The reaction mixture was then concentrated under reduced pressure. The resulting residue was partitioned between ethyl acetate and 1M HCl. The aqueous layer was extracted an additional 3× ethyl acetate, and the combined organics were washed with brine, dried over sodium sulfate and concentrated. The resulting crude material was dissolved in 0.1 mL dichloromethane and 0.2 mL TFA and stirred at room temperature for 1 hour. The volatiles were removed and the crude material was dissolved in 1:1 DMSO/methanol, filtered, and purified by reverse phase HPLC (1-70% ACN in water, HCl modifier, 15 min run) and pure fractions were selected to give the corresponding (11R)-6-(2,6-dimethylphenyl)-11-(2,2-dimethylpropyl)-12-[(3-hydroxy-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (5.2 mg, 4%). ESI-MS m/z calc. 601.2359, found 602.5 (M+1)⁺; Retention time: 1.28 minutes; LC method A.

### Example 41: Preparation of Compound 55

### Step 1: 3-[[4-(2,6-Dimethylphenyl)-6-[(2R)-2-[(4,6-dimethyl-2-pyridyl)methylamino]-4,4-dimethyl-pentoxy]pyrimidin-2-yl]sulfamoyl]benzoic acid

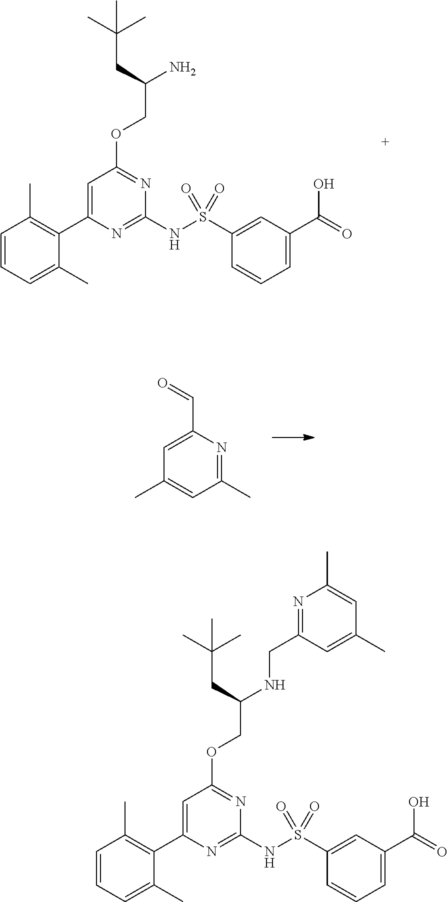

3-[[4-[(2R)-2-amino-4,4-dimethyl-pentoxy]-6-(2,6-dimethylphenyl)pyrimidin-2-yl]sulfamoyl]benzoic acid (hydrochloride salt) (71 mg, 0.1293 mmol), 4,6-dimethylpyridine-2-carbaldehyde (17.9 mg, 0.1324 mmol), and sodium triacetoxyborohydride (38.1 mg, 0.1798 mmol) were combined in DCM (400 μL) and stirred at room temperature for 60 min. The reaction was quenched with aqueous HCl (175 μL of 1 M, 0.1750 mmol), diluted with 1:1 MeOH/DMSO (1 mL) and purified by reverse-phase HPLC utilizing a gradient of 1-99% acetonitrile in 5 mM aqueous HCl to yield 3-[[4-(2,6-dimethylphenyl)-6-[(2R)-2-[(4,6-dimethyl-2-pyridyl)methylamino]-4,4-dimethyl-pentoxy]pyrimidin-2-yl]sulfamoyl]benzoic acid (Dihydrochloride salt) (48.6 mg, 53%) as a white solid. ESI-MS m/z calc. 631.28284, found 632.5 (M+1)⁺; Retention time: 0.53 minutes. ¹H NMR (400 MHz, DMSO-d₆) δ 8.45 (t, J 1.8 Hz, 1H), 8.17-8.09 (m, 2H), 7.68 (t, J 7.8 Hz, 1H), 7.30-7.20 (m, 2H), 7.20-7.09 (m, 3H), 6.33 (s, 1H), 4.48-4.42 (m, 1H), 4.36-4.27 (m, 3H), 3.65-3.59 (m, 1H), 2.46 (s, 3H), 2.30 (s, 3H), 2.01 (s, 6H), 1.74-1.63 (m, 2H), 0.92 (s, 9H). (LC method D).

### Step 2: (11R)-6-(2,6-Dimethylphenyl)-11-(2,2-dimethylpropyl)-12-[(4,6-dimethylpyridin-2-yl)methyl]-9-oxa-2λ⁶-thia-3,5,12,19-tetraazatricyclo[12.3.1.14,8]nonadeca-1(18),4,6,8(19),14,16-hexaene-2,2,13-trione (Compound 55)

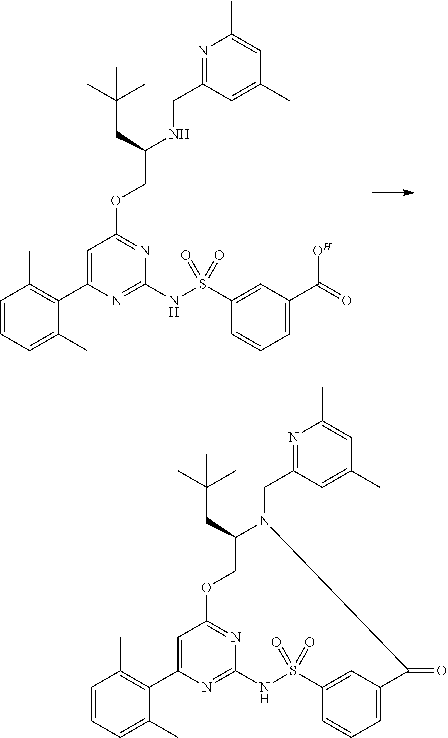 [SMILES: Cc1cc(C)nc(CN[C@@H](COc2cc(-c3c(C)cccc3C)nc(NS(=O)(=O)c3cccc(C(=O)O)c3)n2)CC(C)(C)C)c1 ; Cc1cc(C)nc(CN2C(=O)c3cccc(c3)S(=O)(=O)Nc3nc(cc(-c4c(C)cccc4C)n3)OC[C@H]2CC(C)(C)C)c1]

3-[[4-(2,6-dimethylphenyl)-6-[(2R)-2-[(4,6-dimethyl-2-pyridyl)methylamino]-4,4-dimethyl-pentoxy]pyrimidin-2-yl]sulfamoyl]benzoic acid (Dihydrochloride salt) (44.6 mg, 0.06329 mmol) and CDMT (17.7 mg, 0.1008 mmol) were combined in DMF (1.5 mL) and cooled in an ice bath. 4-methylmorpholine (35 μL, 0.3183 mmol) was added and the reaction was allowed to warm to room temperature and stir for 3 h. The reaction was filtered and purified by reverse-phase HPLC utilizing a gradient of 10-99% acetonitrile in 5 mM aqueous HCl to yield (11R)-6-(2,6-dimethylphenyl)-11-(2,2-dimethylpropyl)-12-[(4,6-dimethylpyridin-2-yl)methyl]-9-oxa-2λ⁶-thia-3,5,12,19-tetraazatricyclo[12.3.1.14,8]nonadeca-1(18),4,6,8(19),14,16-hexaene-2,2,13-trione (hydrochloride salt) (18.2 mg, 44%) as a white solid. ESI-MS m/z calc. 613.2723, found 614.4 (M+1)⁺; Retention time: 1.31 minutes. ¹H NMR (400 MHz, Chloroform-d) δ 8.66 (t, J 1.8 Hz, 1H), 8.17 (dt, J 8.2, 1.3 Hz, 1H), 7.92 (dt, J 7.9, 1.4 Hz, 1H), 7.77-7.72 (m, 1H), 7.76-7.67 (m, 1H), 7.36 (s, 1H), 7.25-7.19 (m, 1H), 7.10-7.04 (m, 2H), 6.27 (s, 1H), 5.53 (d, J 16.1 Hz, 1H), 5.39 (d, J 16.1 Hz, 1H), 5.02 (dd, J 10.2, 4.3 Hz, 1H), 4.70 (t, J 11.0 Hz, 1H), 4.33-4.23 (m, 1H), 2.99 (s, 3H), 2.59 (s, 3H), 2.10-2.02 (m, 1H), 2.02 (s, 6H), 1.65 (dd, J 15.9, 1.7 Hz, 1H), 0.59 (s, 9H), (LC method A).

### Example 42: Preparation of Compound 56

### Step 1: 3,5-Dimorpholinopyridine-2-carbaldehyde

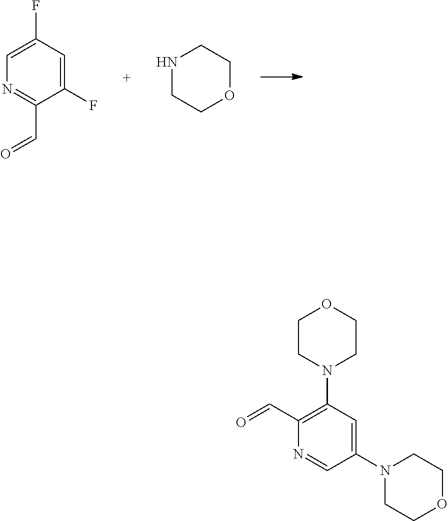 [SMILES: O=Cc1ncc(F)cc1F ; C1COCCN1 ; O=Cc1ncc(N2CCOCC2)cc1N1CCOCC1]

3,5-difluoropyridine-2-carbaldehyde (100 mg, 0.6989 mmol) was combined with morpholine (approximately 243.6 mg, 243.8 μL, 2.796 mmol) and potassium carbonate (approximately 482.9 mg, 3.494 mmol) in DMF (1.165 mL) in a screwcap vial. The reaction mixture was heated to 110° C. for 3 hours. After cooling to room temperature, the reaction mixture was diluted with methanol and filtered through Celite. Several drops of water were added to the filtrate, and it was concentrated under reduced pressure. The resulting crude material was purified by chromatography on silica gel, eluting with a 0-10% methanol in dichloromethane gradient. Fractions containing product were concentrated to give 3,5-dimorpholinopyridine-2-carbaldehyde (152.3 mg, 79%).ESI-MS m/z calc. 277.14264, found 278.6 (M+1)⁺; Retention time: 0.28 minutes; LC method D.

### Step 2: (11R)-6-(2,6-Dimethylphenyl)-11-(2,2-dimethylpropyl)-12-[(3,5-dimorpholino-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 56)

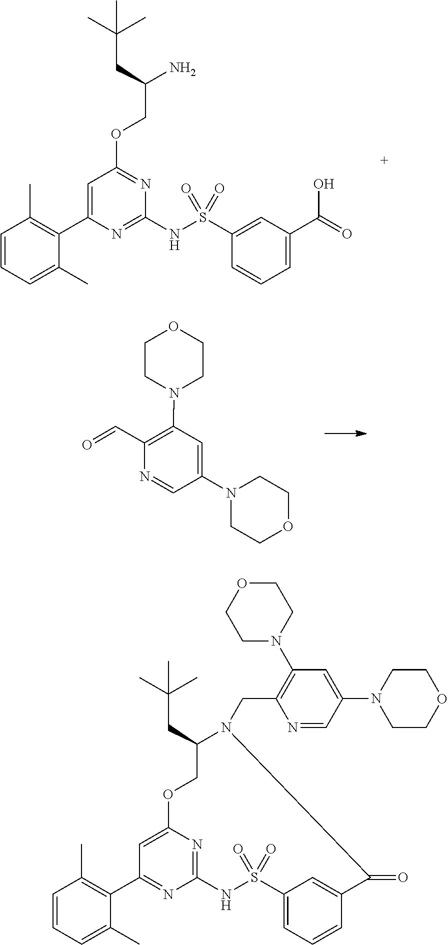

3-[[4-[(2R)-2-amino-4,4-dimethyl-pentoxy]-6-(2,6-dimethylphenyl)pyrimidin-2-yl]sulfamoyl]benzoic acid (hydrochloride salt) (50 mg, 0.09106 mmol) was combined with 3,5-dimorpholinopyridine-2-carbaldehyde (approximately 30.31 mg, 0.1093 mmol) in DCM (151.8 μL) and stirred at room temperature for 15 minutes. Sodium triacetoxyborohydride (approximately 19.30 mg, 0.09106 mmol) (1 equivalent) was added, followed by additional sodium triacetoxyborohydride (approximately 57.90 mg, 0.2732 mmol) (3 equivalents) 20 minutes later. The reaction was stirred at room temperature for an additional 30 minutes. It was then quenched with several drops of 1M HCl, diluted with 1:1 DMSO/methanol, filtered, and purified by reverse phase HPLC (1-70% ACN in water, HCl modifier, 15 min run) to give the reductive amination product. It was dissolved in DMF (1.5 mL), and cooled to 0° C. N-methylmorpholine (approximately 55.27 mg, 60.08 μL, 0.5464 mmol) was added, followed by CDMT (approximately 20.79 mg, 0.1184 mmol). After 30 minutes the reaction mixture was warmed to room temperature and stirred for an additional 2 hours at room temperature. The reaction mixture was then filtered and purified by reverse phase HPLC (1-70% ACN in water, HCl modifier). Pure fractions were dried to give (11R)-6-(2,6-dimethylphenyl)-11-(2,2-dimethylpropyl)-12-[(3,5-dimorpholino-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (15.4 mg, 22%). ESI-MS m/z calc. 755.3465, found 756.6 (M+1)⁺; Retention time: 1.43 minutes; LC method A.

### Example 43: Preparation of Compound 57

### Step 1: 3-Methyl-5-morpholino-pyridine-2-carbaldehyde

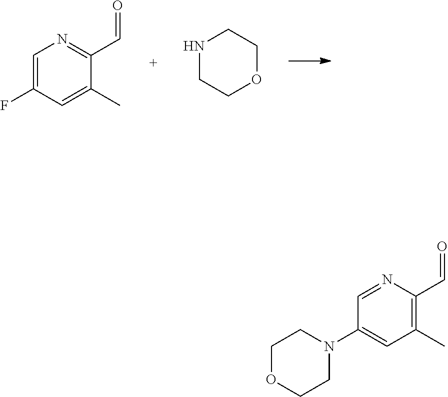

5-fluoro-3-methyl-pyridine-2-carbaldehyde (100 mg, 0.7188 mmol) was combined with morpholine (125 μL, 1.433 mmol) and potassium carbonate (300 mg, 2.171 mmol) in DMF (1 mL). The reaction mixture was heated to 110° C. for 2 hours. After cooling to room temperature the reaction mixture was filtered through Celite, eluting with methanol. Several drops of water were added to the filtrate and it then was concentrated under reduced pressure. The resulting crude material was purified by chromatography on silica gel (eluting with a 0-10% methanol in DCM gradient) to give as a white solid, 3-methyl-5-morpholino-pyridine-2-carbaldehyde (94.5 mg, 64%) ESI-MS m/z calc. 206.10553, found 207.2 (M+1)⁺; Retention time: 0.25 minutes (LC method D).

### Step 2: (11R)-6-(2,6-Dimethylphenyl)-11-(2,2-dimethylpropyl)-12-[(3-methyl-5-morpholino-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 57)

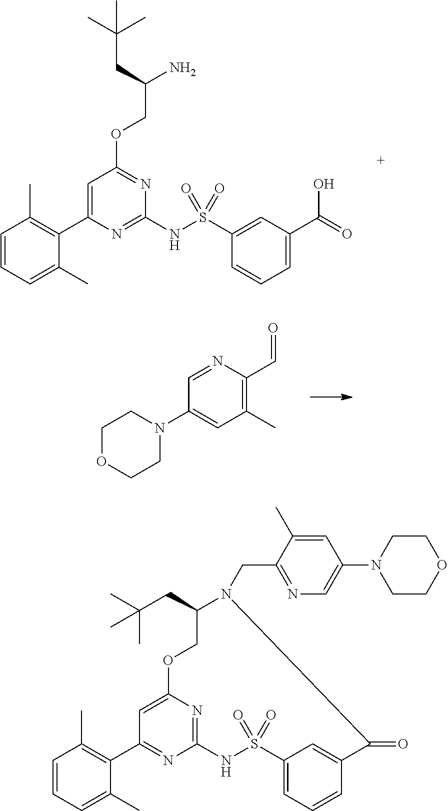

3-[[4-[(2R)-2-amino-4,4-dimethyl-pentoxy]-6-(2,6-dimethylphenyl)pyrimidin-2-yl]sulfamoyl]benzoic acid (hydrochloride salt) (50 mg, 0.09106 mmol) was combined with the 3-methyl-5-morpholino-pyridine-2-carbaldehyde (approximately 22.54 mg, 0.1093 mmol) in DCM (0.3 mL) and stirred at room temperature for 15 minutes. Sodium triacetoxyborohydride (approximately 19.30 mg, 0.09106 mmol) (1 equivalent) was added, followed by additional sodium triacetoxyborohydride (approximately 57.90 mg, 0.2732 mmol) (3 equivalents) 20 minutes later. The reaction was stirred at room temperature for an additional 30 minutes. The reaction mixture was then quenched with several drops of 1M HCl, diluted with 1:1 DMSO/methanol, filtered, and purified by reverse phase HPLC (1-70% ACN in water, HCl modifier, 15 min run) to give the reductive amination product. It was dissolved in DMF (1.5 mL), and cooled to 0° C. N-methylmorpholine (approximately 55.27 mg, 60.08 μL, 0.5464 mmol) was added, followed by CDMT (approximately 20.79 mg, 0.1184 mmol). After 30 minutes the reaction mixture was warmed to room temperature and stirred for an additional 3 hours at room temperature. The reaction mixture was then filtered and purified by reverse phase HPLC (1-70% ACN in water, HCl modifier). Pure fractions were dried to give (11R)-6-(2,6-dimethylphenyl)-11-(2,2-dimethylpropyl)-12-[(3-methyl-5-morpholino-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (11.8 mg, 19%) ESI-MS m/z calc. 684.3094, found 685.5 (M+1)⁺; Retention time: 1.42 minutes; LC method A.

### Example 44: Preparation of Compound 58

### Step 1: 3-[[4-(2,6-Dimethylphenyl)-6-[(2R)-2-(1-isoquinolylmethylamino)-4,4-dimethyl-pentoxy]pyrimidin-2-yl]sulfamoyl]benzoic acid

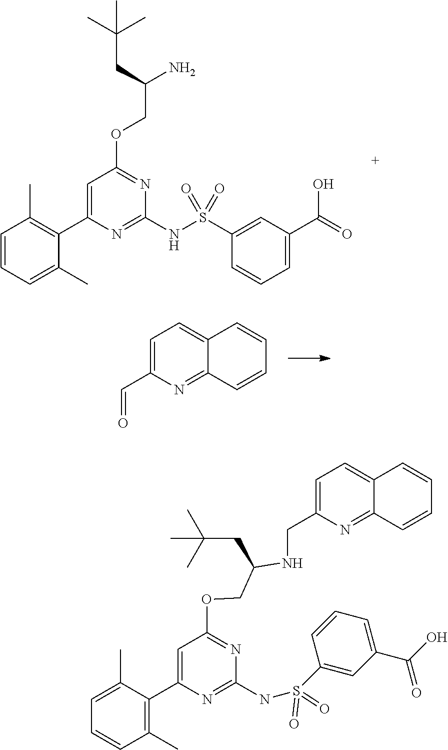

In a 4 mL vial, to a stirred heterogeneous mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (70 mg, 0.1275 mmol) in anhydrous dichloromethane (1.0 mL) were added quinoline-2-carbaldehyde (21 mg, 0.1336 mmol), and glacial acetic acid (10 μL, 0.1758 mmol), in that order. The vial was purged with nitrogen briefly and capped and stirred for 5 min, then sodium triacetoxyborohydride (150 mg, 0.7077 mmol) was added, followed by addition of DIEA (70 μL, 0.4019 mmol), and the capped vial was allowed to stir at ambient temperature for 30 min. Then methanol (0.3 mL) and water (0.2 mL) were added to the reaction and the volatiles were removed under reduced pressure and the residue was taken up in DMSO (1.5 mL), micro-filtered, and purified by reverse-phase HPLC, C₁₈ column, 1-99% acetonitrile in water over 15 min, HCl as modifier, single injection on small column) to furnish 3-[[4-(2,6-dimethylphenyl)-6-[(2R)-4,4-dimethyl-2-(2-quinolylmethylamino)pentoxy]pyrimidin-2-yl]sulfamoyl]benzoic acid (Dihydrochloride salt) (58 mg, 63%), obtained as a pinkish solid. ¹H NMR (400 MHz, DMSO-d₆) δ 13.42 (s, 1H), 9.55 (s, 2H), 8.50-8.42 (m, 2H), 8.20-8.10 (m, 2H), 8.09-7.95 (m, 2H), 7.80 (ddd, J 8.5, 6.9, 1.6 Hz, 1H), 7.72-7.59 (m, 3H), 7.26 (t, J 7.6 Hz, 1H), 7.13 (d, J 7.7 Hz, 2H), 6.35 (s, 1H), 4.75-4.60 (m, 2H), 4.53 (d, J 11.8 Hz, 1H), 4.37 (s, 1H), 3.77 (s, 1H), 1.99 (s, 6H), 1.85-1.68 (m, 2H), 0.94 (s, 9H). ESI-MS m/z calc. 653.2672, found 654.3 (M+1)⁺; Retention time: 1.33 minutes (LC method A).

### Step 2: (11R)-6-(2,6-Dimethylphenyl)-11-(2,2-dimethylpropyl)-2,2-dioxo-12-(2-quinolylmethyl)-9-oxa-2λ⁶-thia-3,5,12,19-tetrazatricyclo[12.3.1.14,8]nonadeca-1(18),4(19),5,7,14,16-hexaen-13-one (Compound 58)

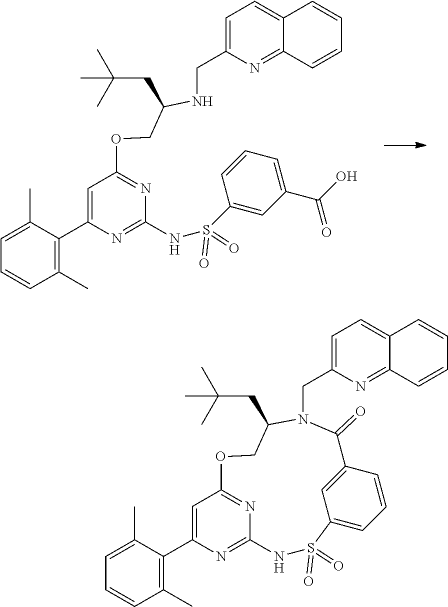 [SMILES: Cc1cccc(C)c1-c1cc(OC[C@@H](CC(C)(C)C)NCc2ccc3ccccc3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccc3ccccc3n1)[C@H](CC(C)(C)C)CO2]

In a 4 mL vial, to a stirred solution of 3-[[4-(2,6-dimethylphenyl)-6-[(2R)-4,4-dimethyl-2-(2-quinolylmethylamino)pentoxy]pyrimidin-2-yl]sulfamoyl]benzoic acid (Dihydrochloride salt) (50 mg, 0.06880 mmol) in anhydrous DMF (2.5 mL) was added [dimethylamino(triazolo[4,5-b]pyridin-3-yloxy)methylene]-dimethyl-ammonium (Phosphorus Hexafluoride Ion) (50 mg, 0.1315 mmol) (HATU), followed by addition of DIEA (70 μL, 0.4019 mmol) at ambient temperature. The vial was briefly purged with nitrogen and the capped reaction was allowed to stir at ambient temperature for 30 min. The reaction was micro-filtered, and purified by preparative reverse-phase HPLC (1-99% acetonitrile in water over 15 min, HCl as modifier) to give (11R)-6-(2,6-dimethylphenyl)-11-(2,2-dimethylpropyl)-2,2-dioxo-12-(2-quinolylmethyl)-9-oxa-2λ⁶-thia-3,5,12,19-tetrazatricyclo[12.3.1.14,8]nonadeca-1(18),4(19),5,7,14,16-hexaen-13-one (hydrochloride salt) (34 mg, 73%) as a white solid. ¹H NMR (400 MHz, DMSO-d₆) δ 13.05 (s, 1H), 9.07 (s, 1H), 8.36 (d, J 8.5 Hz, 1H), 8.08 (d, J 8.4 Hz, 1H), 7.98 (dd, J=8.3, 1.4 Hz, 1H), 7.95 (s, 1H), 7.77 (ddd, J=8.4, 6.9, 1.5 Hz, 1H), 7.68 (s, 2H), 7.62-7.54 (m, 2H), 7.25 (t, J 7.7 Hz, 1H), 7.12 (d, J 7.6 Hz, 2H), 6.38 (s, 1H), 5.65 (d, J 10.2 Hz, 1H), 5.14 (d, J 16.7 Hz, 1H), 4.80 (d, J 16.7 Hz, 1H), 4.15 (t, J 11.1 Hz, 1H), 4.07-3.89 (m, 1H), 2.22-1.88 (m, 6H), 1.84 (dd, J 15.1, 8.7 Hz, 1H), 1.48 (d, J 14.9 Hz, 1H), 0.63 (s, 9H). ESI-MS m/z calc. 635.25665, found 636.3 (M+1)⁺; Retention time: 1.73 minutes (LC method A).

### Example 45: Preparation of Compound 59

### Step 1: 3-[[4-[(2R)-4,4-Dimethyl-2-[(1-methyl-2-oxo-3-pyridyl)methylamino]pentoxy]-6-(2,6-dimethylphenyl)pyrimidin-2-yl]sulfamoyl]benzoic acid

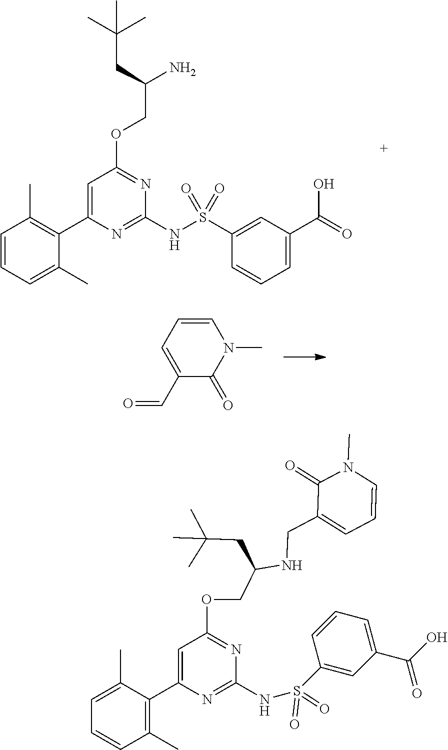

In a 4 mL vial, to a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (65 mg, 0.1184 mmol) in anhydrous dichloromethane (0.6 mL) were added 1-methyl-2-oxo-pyridine-3-carbaldehyde (17 mg, 0.1240 mmol), and glacial acetic acid (10 μL, 0.1758 mmol), in that order. The vial was purged with nitrogen briefly and capped and allowed to stir at ambient temperature for 10 min. Then sodium triacetoxyborohydride (Sodium salt) (150 mg, 0.6385 mmol) and DIEA (70 μL, 0.4019 mmol), in that order. After stirring for another 15 min, methanol (0.3 mL) and water (0.2 mL) were added to the reaction and the volatiles were removed under reduced pressure and the residue was purified by reverse-phase HPLC (1-99% acetonitrile in water over 15 min, 5 mM HCl as modifier) to furnish 3-[[4-[(2R)-4,4-dimethyl-2-[(1-methyl-2-oxo-3-pyridyl)methylamino]pentoxy]-6-(2,6-dimethylphenyl)pyrimidin-2-yl]sulfamoyl]benzoic acid (hydrochloride salt) (40 mg, 50%) as a white solid. It was used in the subsequent reaction. ¹H NMR (400 MHz, DMSO-d₆) δ 13.30 (s, 1H), 9.00 (q, J 5.9, 5.3 Hz, 2H), 8.45 (d, J 1.8 Hz, 1H), 8.13 (dt, J=7.9, 2.1 Hz, 2H), 7.84 (dd, J=6.8, 2.0 Hz, 1H), 7.75-7.62 (m, 2H), 7.27 (t, J 7.6 Hz, 1H), 7.13 (d, J 7.6 Hz, 2H), 6.41-6.27 (m, 2H), 4.40 (dd, J 12.5, 3.0 Hz, 1H), 4.29 (dd, J=12.4, 6.7 Hz, 1H), 4.13-4.03 (m, 2H), 3.57 (s, 1H), 3.48 (s, 3H), 2.02 (s, 6H), 1.76 (d, J 15.4 Hz, 1H), 1.64 (dd, J 14.8, 8.4 Hz, 1H), 0.94 (s, 9H). ESI-MS m/z calc. 633.2621, found 634.5 (M+1)⁺; Retention time: 0.38 minutes (LC method A).

### Step 2: (11R)-6-(2,6-Dimethylphenyl)-11-(2,2-dimethylpropyl)-12-[(1-methyl-2-oxo-3-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 59)

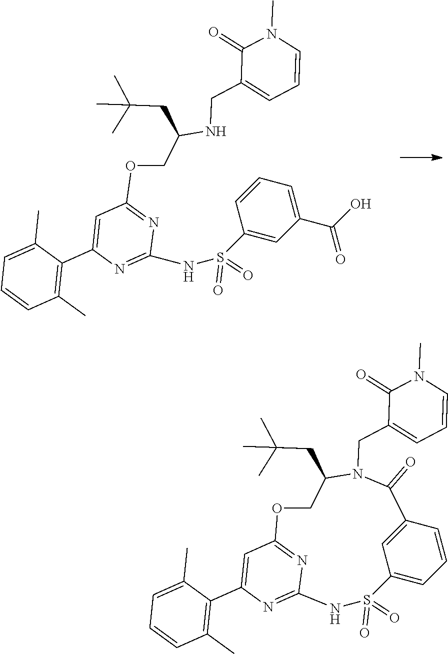 [SMILES: Cc1cccc(C)c1-c1cc(OC[C@@H](CC(C)(C)C)NCc2cccn(C)c2=O)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cccn(C)c1=O)[C@H](CC(C)(C)C)CO2]

In a 4 mL vial, to a stirred solution of 3-[[4-[(2R)-4,4-dimethyl-2-[(1-methyl-2-oxo-3-pyridyl)methylamino]pentoxy]-6-(2,6-dimethylphenyl)pyrimidin-2-yl]sulfamoyl]benzoic acid (hydrochloride salt) (32 mg, 0.04775 mmol) in anhydrous DMF (1.7 mL) was added [dimethylamino(triazolo[4,5-b]pyridin-3-yloxy)methylene]-dimethyl-ammonium (Phosphorus Hexafluoride Ion) (37 mg, 0.09731 mmol) (HATU), followed by addition of DIEA (50 μL, 0.2871 mmol) at ambient temperature. The vial was briefly purged with nitrogen and the capped reaction was allowed to stir at ambient temperature for 2 h. The product was purified by preparative reverse-phase HPLC (1-99% acetonitrile in water over 15 min, 5 mM HCl as modifier) to furnish (11R)-6-(2,6-dimethylphenyl)-11-(2,2-dimethylpropyl)-12-[(1-methyl-2-oxo-3-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (16 mg, 54%) as a white solid. ¹H NMR (400 MHz, DMSO-d₆) δ 13.07 (s, 1H), 8.54 (s, 1H), 7.96 (d, J 6.4 Hz, 1H), 7.72 (s, 1H), 7.68 (dd, J 6.7, 1.9 Hz, 2H), 7.43 (dd, J 6.9, 1.9 Hz, 1H), 7.26 (t, J 7.7 Hz, 1H), 7.12 (d, J 7.8 Hz, 2H), 6.41 (s, 1H), 6.29 (t, J 6.8 Hz, 1H), 5.12 (dd, J 10.8, 4.4 Hz, 1H), 4.57 (d, J 16.2 Hz, 1H), 4.31 (d, J 11.2 Hz, 1H), 4.26 (d, J 16.0 Hz, 1H), 4.07-3.96 (m, 1H), 3.50 (s, 3H), 2.27-1.83 (m, 6H), 1.77 (dd, J 15.3, 8.5 Hz, 1H), 1.38 (d, J 15.1 Hz, 1H), 0.52 (s, 9H). ESI-MS m/z calc. 615.2515, found 616.3 (M+1)⁺; Retention time: 1.66 minutes (LC method A).

### Example 46: Preparation of Compound 60

### Step 1: (11R)-6-(2,6-Dimethylphenyl)-11-(2,2-dimethylpropyl)-12-[(6-fluoro-3-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

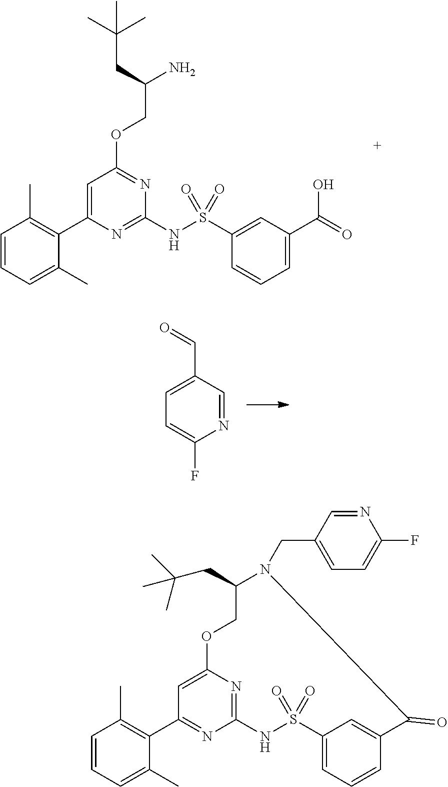 [SMILES: Cc1cccc(C)c1-c1cc(OC[C@H](N)CC(C)(C)C)nc(NS(=O)(=O)c2cccc(C(=O)O)c2)n1 ; O=Cc1ccc(F)nc1 ; Cc1cccc(C)c1-c1cc2nc(n1)NS(=O)(=O)c1cccc(c1)C(=O)N(Cc1ccc(F)nc1)[C@H](CC(C)(C)C)CO2]

3-[[4-[(2R)-2-amino-4,4-dimethyl-pentoxy]-6-(2,6-dimethylphenyl)pyrimidin-2-yl]sulfamoyl]benzoic acid (hydrochloride salt) (330 mg, 0.6010 mmol) was combined with the 6-fluoropyridine-3-carbaldehyde (approximately 90.22 mg, 0.7212 mmol) in DCM (1.202 mL) and stirred at room temperature for 15 minutes. Sodium triacetoxyborohydride (approximately 127.4 mg, 0.6010 mmol) (1 equivalent) was added then added, followed by an additional sodium triacetoxyborohydride (approximately 382.1 mg, 1.803 mmol) (3 equivalents) 15 minutes later. The reaction mixture was then stirred at room temperature for 2 hours. After this time the reaction mixture was quenched into 1M HCl and extracted 4× with ethyl acetate. The combined organics were washed with brine, dried over sodium sulfate and concentrated. The resulting crude material was purified by reverse phase HPLC (1-70% ACN in water, HCl modifier, 15 min run) to give the corresponding reductive amination product. It was combined with N-methylmorpholine (approximately 243.2 mg, 264.3 μL, 2.404 mmol) in DMF (8 mL) and cooled to 0° C. Solid CDMT (approximately 158.3 mg, 0.9015 mmol) was added and the reaction was stirred at 0° C. for 1 hour then room temperature for an additional 4 hours. The reaction mixture was then concentrated under reduced pressure. The crude material was dissolved in 1:1 DMSO/methanol, filtered, and purified by reverse phase HPLC (1-70% ACN in water, HCl modifier, 15 min run) and pure fractions were selected to give the corresponding (11R)-6-(2,6-dimethylphenyl)-11-(2,2-dimethylpropyl)-12-[(6-fluoro-3-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (125 mg, 34%) ESI-MS m/z calc. 603.23157, found 604.5 (M+1)⁺; Retention time: 0.68 minutes; LC method A.

### Step 2: (11R)-6-(2,6-Dimethylphenyl)-11-(2,2-dimethylpropyl)-12-[(6-morpholino-3-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 60)

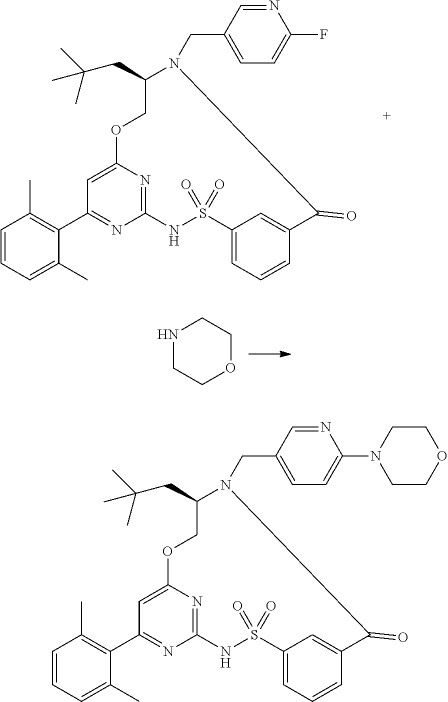 [SMILES: Cc1cccc(C)c1-c1cc2nc(n1)NS(=O)(=O)c1cccc(c1)C(=O)N(Cc1ccc(F)nc1)[C@H](CC(C)(C)C)CO2 ; C1COCCN1 ; Cc1cccc(C)c1-c1cc2nc(n1)NS(=O)(=O)c1cccc(c1)C(=O)N(Cc1ccc(N3CCOCC3)nc1)[C@H](CC(C)(C)C)CO2]

(11R)-6-(2,6-dimethylphenyl)-11-(2,2-dimethylpropyl)-12-[(6-fluoro-3-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (18 mg, 0.02982 mmol) was combined with morpholine (10 μL, 0.1147 mmol), and potassium carbonate (16 mg, 0.1158 mmol), in DMF (200 μL) and heated to 110° C. for 1 hour. Additional morpholine (30 μL, 0.3440 mmol), potassium carbonate (17 mg, 0.1230 mmol), as well as DMSO (200 mL) were added and the reaction temperature was increased to 125° C. After 18 hours, the reaction mixture was cooled to room temperature, diluted with methanol, filtered, and purified by reverse phase HPLC (1-70% ACN in water, HCl modifier, 15 min run). A pure faction was dried to give (11R)-6-(2,6-dimethylphenyl)-11-(2,2-dimethylpropyl)-12-[(6-morpholino-3-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (7.5 mg, 36%). ESI-MS m/z calc. 670.29376, found 671.5 (M+1)⁺; Retention time: 1.3 minutes (LC method A).

### Example 47: Preparation of Compound 61

### Step 1: (11R)-12-[(2-Amino-4-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 61)

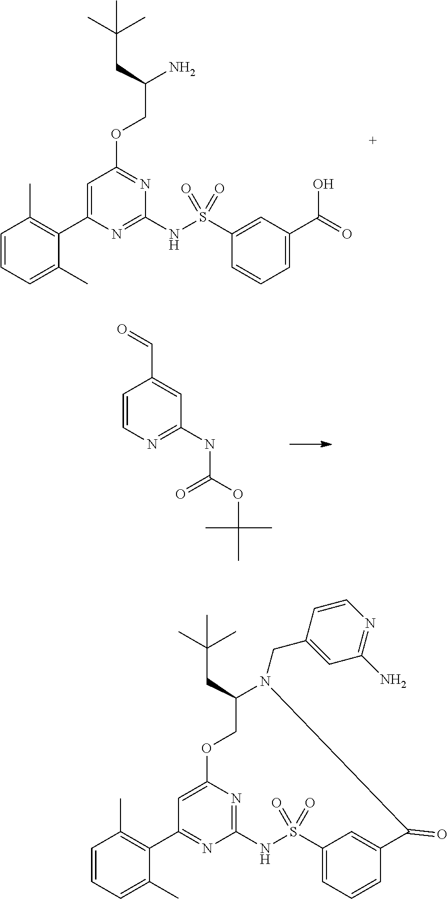 [SMILES: Cc1cccc(C)c1-c1cc(OC[C@H](N)CC(C)(C)C)nc(NS(=O)(=O)c2cccc(C(=O)O)c2)n1 ; CC(C)(C)OC(=O)Nc1cc(C=O)ccn1 ; Cc1cccc(C)c1-c1cc2nc(n1)NS(=O)(=O)c1cccc(c1)C(=O)N(Cc1ccnc(N)c1)[C@H](CC(C)(C)C)CO2]

3-[[4-[(2R)-2-amino-4,4-dimethyl-pentoxy]-6-(2,6-dimethylphenyl)pyrimidin-2-yl]sulfamoyl]benzoic acid (hydrochloride salt) (80 mg, 0.1457 mmol) was combined with tert-butyl N-(4-formyl-2-pyridyl)carbamate (approximately 38.85 mg, 0.1748 mmol) in DCM (0.5 mL) at room temperature. After stirring at room temperature for 15 minutes, sodium triacetoxyborohydride (approximately 92.64 mg, 0.4371 mmol) was added in two portions over 15 minutes. The reaction mixture was stirred for 3 hours at room temperature. The reaction mixture was then partitioned between 1M HCl and ethyl acetate and the layers were separated. The aqueous was extracted an additional 2× with ethyl acetate and the combined organics were washed with brine, dried over sodium sulfate and concentrated. This crude material was then dissolved in 1:1 DMSO/methanol, filtered, and purified by reverse phase HPLC (1-70% ACN in water, HCl modifier, 15 min run). The product partially Boc-deprotected while drying, but it was used in the subsequent step without further purification

The product from step 1 was dissolved in DMF (5 mL) and added dropwise to a stirring solution of HATU (approximately 55.40 mg, 0.1457 mmol) and DIPEA (approximately 94.15 mg, 126.9 μL, 0.7285 mmol) in DMF (3 mL), and stirred for 16 hours at room temperature. The reaction mixture was partially concentrated, then partitioned between 1M HCl and ethyl acetate. The aqueous layer was extracted with an additional 2× with ethyl acetate, and the combined organics were washed with brine, dried over sodium sulfate and concentrated. The crude material was treated with HCl (approximately 728.5 μL of 4 M, 2.914 mmol) in dioxane and stirred for 90 minutes at room temperature. The volatiles were removed and the crude material was then dissolved in 1:1 DMSO/methanol, filtered, and purified by reverse phase HPLC (1-99% ACN in water, 30 min run) to give the corresponding (11R)-12-[(2-amino-4-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (2.2 mg, 2%) ESI-MS m/z calc. 600.2519, found 601.6 (M+1)⁺; Retention time: 1.26 minutes; LC method A.

### Example 48: Preparation of Compound 62

### Step 1: 3-[[4-[(2R)-2-[(6-Chloropyrazin-2-yl)methylamino]-4,4-dimethyl-pentoxy]-6-(2,6-dimethylphenyl)pyrimidin-2-yl]sulfamoyl]benzoic acid

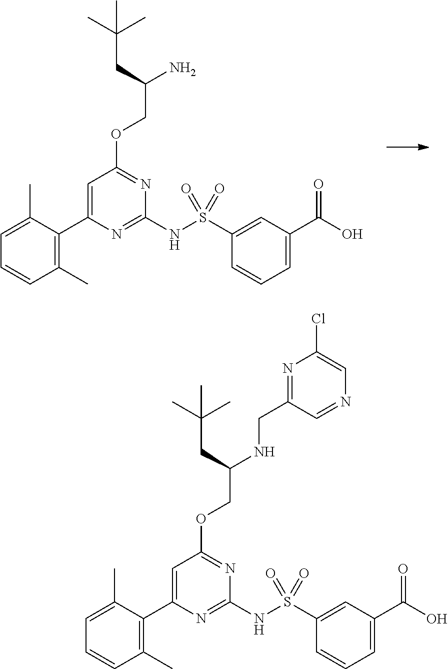 [SMILES: Cc1cccc(C)c1-c1cc(OC[C@H](N)CC(C)(C)C)nc(NS(=O)(=O)c2cccc(C(=O)O)c2)n1 ; Cc1cccc(C)c1-c1cc(OC[C@@H](CC(C)(C)C)NCc2cncc(Cl)n2)nc(NS(=O)(=O)c2cccc(C(=O)O)c2)n1]

3-[[4-[(2R)-2-amino-4,4-dimethyl-pentoxy]-6-(2,6-dimethylphenyl)pyrimidin-2-yl]sulfamoyl]benzoic acid (hydrochloride salt) (1.00 g, 1.730 mmol) and 6-chloropyrazine-2-carbaldehyde (296 mg, 2.077 mmol) were combined and dissolved/suspended in dichloromethane (22 mL). After stirring at room temperature for 30 minutes, sodium triacetoxyborohydride (Sodium salt) (1.47 g, 6.936 mmol) was added in four equal portions separated by fifteen-minute intervals. Aqueous 1 M HCl was added to quench the reaction. After brief stirring, the mixture was diluted with EtOAc (75 mL) and washed with aqueous HCl (1 M, 1×75 mL) and brine (1×75 mL). The organic layer was dried over sodium sulfate, filtered and concentrated under reduced pressure. The product was isolated by silica gel column chromatography eluting with a 0-100% EtOAc/hexane gradient followed by a 0-30% MeOH/DCM gradient. Fractions containing the desired product were combined and concentrated after the addition of HCl in dioxane (500 μL of 4 M, 2.000 mmol). 3-[[4-[(2R)-2-[(6-Chloropyrazin-2-yl)methylamino]-4,4-dimethyl-pentoxy]-6-(2,6-dimethylphenyl)pyrimidin-2-yl]sulfamoyl]benzoic acid (hydrochloride salt) (1.14 g, 98%) was obtained. ESI-MS m/z calc. 638.2078, found 639.2 (M+1)⁺; Retention time: 1.22 minutes (LC method A).

### Step 2: (11R)-12-[(6-Chloropyrazin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

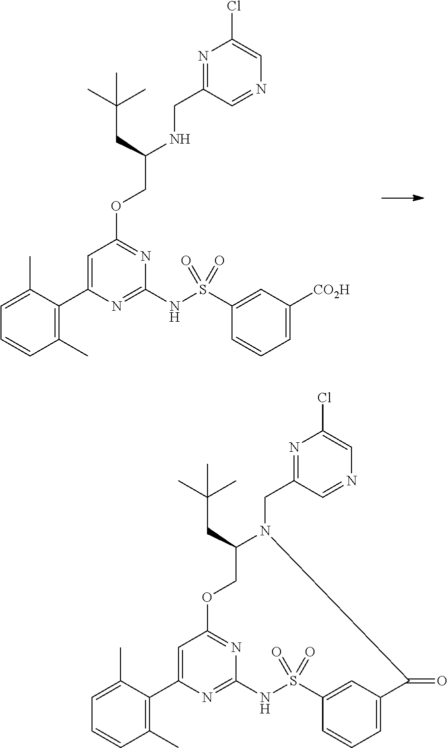 [SMILES: Cc1cccc(C)c1-c1cc(OC[C@@H](CC(C)(C)C)NCc2cncc(Cl)n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cncc(Cl)n1)[C@H](CC(C)(C)C)CO2]

A solution of 3-[[4-[(2R)-2-[(6-chloropyrazin-2-yl)methylamino]-4,4-dimethyl-pentoxy]-6-(2,6-dimethylphenyl)pyrimidin-2-yl]sulfamoyl]benzoic acid (hydrochloride salt) (2.4 g, 3.0905 mmol) in DMF (250 mL) was stirred at 0° C. for 30 min. CDMT (2.72 g, 15.492 mmol) and N-methyl morpholine (1.5640 g, 1.7 mL, 15.463 mmol) were then added at the same temperature. The reaction mixture was stirred at 0° C. for 30 min and was allowed to warm to room temperature and stirred overnight. The reaction was quenched with 1M aqueous HCl (100 mL) and diluted with water (1000 mL). The reaction mixture was extracted with EtOAc (3×200 mL). The combined organic layers were washed with water (2×300 mL) and brine (300 mL). The organic layer was dried over anhydrous sodium sulfate, filtered, and concentrated under reduced pressure. The crude product was purified by flash chromatography (120 g column, dry loaded, eluting from 0 to 80% EtOAc in hexanes in 60 min) yielding (11R)-12-[(6-chloropyrazin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.17 g, 56%) as a white solid. ESI-MS m/z calc. 620.1973, found 621.4 (M+1)⁺; Retention time: 2.63 minutes. ¹H NMR (500 MHz, DMSO) δ 8.78 (s, 1H), 8.69 (s, 1H), 8.63 (s, 1H), 7.95 (d, J 5.7 Hz, 1H), 7.75-7.54 (m, 2H), 7.25 (t, J 7.4 Hz, 1H), 7.12 (d, J 6.4 Hz, 2H), 6.42 (s, 1H), 5.33 (dd, J 10.7, 4.1 Hz, 1H), 4.86 (d, J 16.2 Hz, 1H), 4.71 (d, J 16.2 Hz, 1H), 4.30 (t, J 11.1 Hz, 1H), 4.05-3.93 (m, 1H), 2.26-1.83 (m, 6H), 1.79 (dd, J 15.2, 8.7 Hz, 1H), 1.43 (d, J 14.9 Hz, 1H), 0.55 (s, 9H). LC method W.

### Step 3: (11R)-6-(2,6-Dimethylphenyl)-11-(2,2-dimethylpropyl)-12-({6-[(2,2-dimethylpropyl)(methyl)amino]pyrazin-2-yl}methyl)-9-oxa-2λ⁶-thia-3,5,12,19-tetraazatricyclo[12.3.1.14,8]nonadeca-1(18),4,6,8(19),14,16-hexaene-2,2,13-trione (Compound 62)

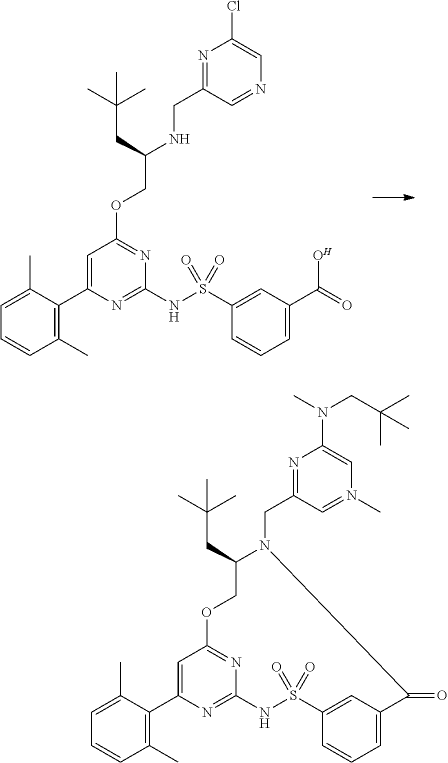

(11R)-12-[(6-chloropyrazin-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (50 mg, 0.08050 mmol) and N,2,2-trimethylpropan-1-amine (24 mg, 0.2372 mmol) were combined and dissolved in DMSO (0.25 mL) with finely ground potassium carbonate (67 mg, 0.4848 mmol). The reaction mixture was stirred at 125° C. overnight. The reaction mixture was diluted with EtOAc (75 mL). It was then washed with water (1×75 mL) and brine (1×75 mL). The organic layer was dried over sodium sulfate, filtered and concentrated under reduced pressure. The product was isolated by UV-triggered reverse-phase HPLC eluting with a 10-99% acetonitrile/water gradient over 15 minutes with 5 mM HCl acid modifier in the aqueous phase. (11R)-6-(2,6-dimethylphenyl)-11-(2,2-dimethylpropyl)-12-({6-[(2,2-dimethylpropyl)(methyl)amino]pyrazin-2-yl}methyl)-9-oxa-2λ⁶-thia-3,5,12,19-tetraazatricyclo[12.3.1.14,8]nonadeca-1(18),4,6,8(19),14,16-hexaene-2,2,13-trione (4.9 mg, 9%) was obtained. ¹H NMR (400 MHz, DMSO-d₆) δ 12.77 (s, 1H), 8.66 (s, 1H), 8.07 (s, 1H), 7.99-7.91 (m, 1H), 7.84 (s, 1H), 7.69 (d, J 5.2 Hz, 2H), 7.26 (t, J 7.6 Hz, 1H), 7.12 (d, J 7.6 Hz, 2H), 6.43 (s, 1H), 5.39 (dd, J 10.8, 4.3 Hz, 1H), 4.72 (d, J 15.7 Hz, 1H), 4.43 (d, J 15.7 Hz, 1H), 4.29 (t, J 11.2 Hz, 1H), 4.09-4.00 (m, 1H), 3.47 (d, J 3.1 Hz, 2H), 3.16 (s, 3H), 2.03 (d, J 27.9 Hz, 6H), 1.77 (dd, J 15.2, 8.9 Hz, 1H), 1.41 (d, J 15.0 Hz, 1H), 0.92 (s, 9H), 0.56 (s, 9H). ESI-MS m/z calc. 685.341, found 686.4 (M+1)⁺; Retention time: 1.93 minutes (LC method A).

### Example 49: Preparation of Compound 63 and Compound 64

### Step 1: Methyl 5,8-dioxaspiro[3.4]octane-2-carboxylate

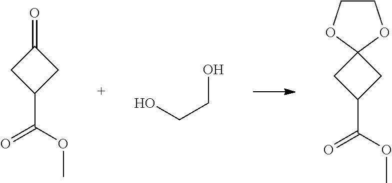

To a solution of methyl 3-oxocyclobutanecarboxylate (9.7 g, 75.707 mmol) in toluene (100 mL) were added ethylene glycol (7.2345 g, 6.5 mL, 116.56 mmol) and p-toluenesulfonic acid hydrate (1.5 g, 7.8857 mmol). The reaction mixture was stirred at reflux (in an oil bath at 140° C.) with a Dean-Stark apparatus for 16 h. The reaction mixture was then cooled down to room temperature and diluted with a saturated aqueous solution of KHCO₃ (100 mL) and the phases were separated. The aqueous layer was washed with EtOAc (3×50 mL) and the combined organic layers were dried over magnesium sulfate, filtered and concentrated in vacuo to afford methyl 5,8-dioxaspiro[3.4]octane-2-carboxylate (13.52 g, 104%) as a pale-yellow oil. Product is contaminated with residual p-TsOH. It was carried through directly into next step. ESI-MS m/z calc. 172.0736, found 173.2 (M+1)⁺; Retention time: 1.35 minutes (LC method X).

### Step 2: 5,8-Dioxaspiro[3.4]octan-2-ylmethanol

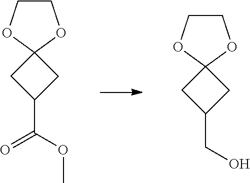

LAH (3 g, 79.042 mmol) was added portionwise (keeping temperature below 30° C.) to a solution of methyl 5,8-dioxaspiro[3.4]octane-2-carboxylate (9.59 g, 55.698 mmol) in THE (150 mL) at 0-5° C. The mixture was stirred at room temperature for 4 h. The reaction mixture was then cooled to 0° C. and water (3 mL) was slowly added followed by 15% aqueous NaOH (3 mL) and finally water (9 mL). The reaction mixture was stirred for 15 min. at room temperature and then it was filtered and rinsed with THF. The filtrate was concentrated in vacuo and the residue was diluted in EtOAc (100 mL) and washed with water (50 mL) and brine (50 mL). More brine (100 mL) was added to the aqueous layer and the product was extracted with EtOAc (3×60 mL). The combined organic layers were dried over magnesium sulfate, filtered and concentrated under reduced pressure to afford 5,8-dioxaspiro[3.4]octan-2-ylmethanol (5.21 g, 65%) as a clear oil. ESI-MS m/z calc. 144.0786, found 145.2 (M+1)⁺; Retention time: 0.78 minutes (LC method X). ¹H NMR (400 MHz, CDCl₃) δ 3.94-3.86 (m, 4H), 3.68 (d, J 6.6 Hz, 2H), 2.50-2.38 (m, 2H), 2.36-2.23 (m, 1H), 2.16-2.07 (m, 2H).

### Step 3: 5,8-Dioxaspiro[3.4]octane-2-carbaldehyde

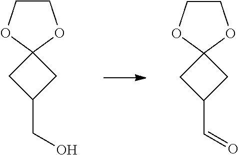 [SMILES: OCC1CC2(C1)OCCO2 ; O=CC1CC2(C1)OCCO2]

To a solution of 5,8-dioxaspiro[3.4]octan-2-ylmethanol (6.17 g, 42.797 mmol) in DCM (125 mL) were added sodium bicarbonate (18 g, 214.27 mmol) and Dess-Martin periodinane (21.8 g, 51.398 mmol). The reaction mixture was stirred at room temperature 3 h. A 10% aqueous solution of sodium bicarbonate (100 mL) was added (strong evolution of gas) followed by a 20% w/w aqueous solution of Na₂S₂O₃ (100 mL). The mixture was vigorously stirred at room temperature for 3 h (until organic phase was clear). The phases were separated and the aqueous layer was washed with DCM (2×70 mL). The combined organic layers were washed with a 10% w/w aqueous solution of Na₂S₂O₃ (100 mL), dried over magnesium sulfate, filtered and concentrated under reduced pressure to afford 5,8-dioxaspiro[3.4]octane-2-carbaldehyde (4.82 g, 79%) as a clear oil. ESI-MS m/z calc. 142.063, found 143.2 (M+1)⁺; Retention time: 0.69 minutes. ¹H NMR (400 MHz, CDCl₃) δ 9.74 (d, J 2.7 Hz, 1H), 3.98-3.86 (m, 4H), 2.98-2.86 (m, 1H), 2.65-2.49 (m, 4H), LC method X.

### Step 4: 3-[[4-(2,6-Dimethylphenyl)-6-[(2R)-2-(5,8-dioxaspiro[3.4]octan-2-ylmethylamino)-4,4-dimethyl-pentoxy]pyrimidin-2-yl]sulfamoyl]benzoic acid

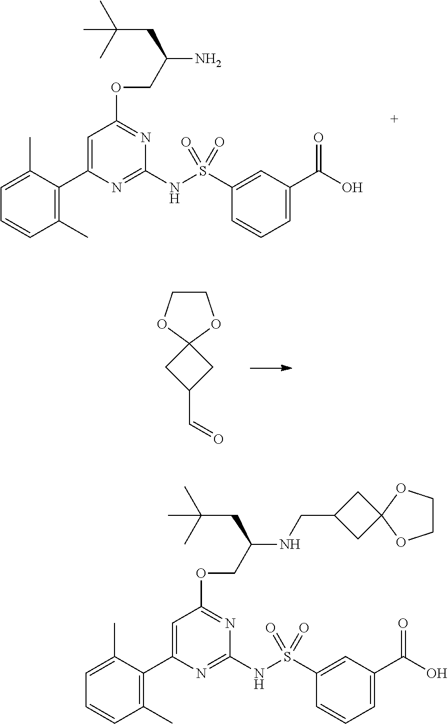 [SMILES: Cc1cccc(C)c1-c1cc(OC[C@H](N)CC(C)(C)C)nc(NS(=O)(=O)c2cccc(C(=O)O)c2)n1 ; O=CC1CC2(C1)OCCO2 ; Cc1cccc(C)c1-c1cc(OC[C@@H](CC(C)(C)C)NCC2CC3(C2)OCCO3)nc(NS(=O)(=O)c2cccc(C(=O)O)c2)n1]

To a stirred solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (10 g, 18.212 mmol) and 5,8-dioxaspiro[3.4]octane-2-carbaldehyde (2.77 g, 19.486 mmol) in DCM (180 mL) was added sodium triacetoxyborohydride (19.3 g, 91.063 mmol). The reaction mixture was stirred at room temperature for 2 h. The reaction was cooled down to 0-5° C. and 1N aqueous HCl (100 mL) was added (strong evolution of gas). DCM was evaporated under reduced pressure and the product was extracted with 2-MeTHF (3×100 mL). The combined organic layers were washed with brine, dried over magnesium sulfate, filtered and evaporated. The crude material (16 g) was triturated in EtOAc (150 mL) at room temperature for 16 h. The resulting solid was filtered on a Buchner funnel. The filter cake was washed with EtOAc (2×50 mL) and dried in vacuo to give 3-[[4-(2,6-dimethylphenyl)-6-[(2R)-2-(5,8-dioxaspiro[3.4]octan-2-ylmethylamino)-4,4-dimethyl-pentoxy]pyrimidin-2-yl]sulfamoyl]benzoic acid (hydrochloride salt) (9.82 g, 80%) as a white solid. ESI-MS m/z calc. 638.2774, found 639.4 (M+1)⁺; Retention time: 1.4 minutes. ¹H NMR (400 MHz, DMSO-d₆) δ 13.40 (br. s., 1H), 9.05-8.66 (m, 2H), 8.46 (s, 1H), 8.14 (t, J 7.5 Hz, 2H), 7.70 (t, J 7.7 Hz, 1H), 7.32-7.21 (m, 1H), 7.13 (d, J 7.3 Hz, 2H), 6.34 (br. s., 1H), 4.40 (d, J 12.5 Hz, 1H), 4.29-4.17 (m, 1H), 3.77 (dd, J 12.7, 5.1 Hz, 4H), 3.57-3.43 (m, 1H), 3.41-3.26 (m, 1H), 3.20-2.98 (m, 2H), 2.43-2.29 (m, 3H), 2.15-1.92 (m, 8H), 1.62 (d, J 4.6 Hz, 2H), 0.93 (s, 9H), LC method X.

### Step 5: (11R)-6-(2,6-Dimethylphenyl)-11-(2,2-dimethylpropyl)-12-(5,8-dioxaspiro[3.4]octan-2-ylmethyl)-2,2-dioxo-9-oxa-2λ⁶-thia-3,5,12,19-tetrazatricyclo[12.3.1.14,8]nonadeca-1(18),4(19),5,7,14,16-hexaen-13-one

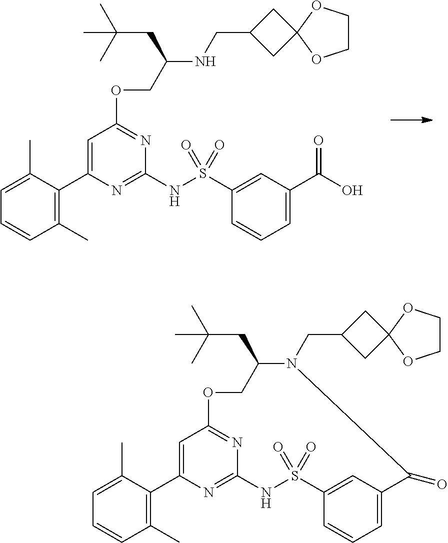 [SMILES: Cc1cccc(C)c1-c1cc(OC[C@@H](CC(C)(C)C)NCC2CC3(C2)OCCO3)nc(NS(=O)(=O)c2cccc(C(=O)O)c2)n1 ; Cc1cccc(C)c1-c1cc2nc(n1)NS(=O)(=O)c1cccc(c1)C(=O)N(CC1CC3(C1)OCCO3)[C@H](CC(C)(C)C)CO2]

To a solution of 3-[[4-(2,6-dimethylphenyl)-6-[(2R)-2-(5,8-dioxaspiro[3.4]octan-2-ylmethylamino)-4,4-dimethyl-pentoxy]pyrimidin-2-yl]sulfamoyl]benzoic acid (hydrochloride salt) (9.8 g, 14.499 mmol) in EtOAc (690 mL) and DMF (150 mL) at 0° C. were added TEA (8.7120 g, 12 mL, 86.095 mmol) and propylphosphonic anhydride 50 wt. % in ethyl acetate (27.7 g, 50% w/w, 43.529 mmol). The reaction mixture was stirred at room temperature for 16 h. The reaction mixture was diluted with 15% brine (600 mL) and the phases were separated. The aqueous layer was washed with EtOAc (2×250 mL) and the combined organic phases were washed with 15% brine (250 mL), dried over magnesium sulfate, filtered and concentrated in vacuo. The residue was purified twice by chromatography on a 120 g silica gel cartridge using a gradient of 30-100% EtOAc in heptanes to provide (11R)-6-(2,6-dimethylphenyl)-11-(2,2-dimethylpropyl)-12-(5,8-dioxaspiro[3.4]octan-2-ylmethyl)-2,2-dioxo-9-oxa-2λ⁶-thia-3, 5,12,19-tetrazatricyclo[12.3.1.14,8]nonadeca-1(18),4(19),5,7,14,16-hexaen-13-one (7.08 g, 79%) as a white solid. The solid was contaminated with 7.1% w/w of ethyl acetate and 6.4% w/w of DMF, based on ¹H NMR. ESI-MS m/z calc. 620.2669, found 621.2 (M+1)⁺; Retention time: 1.86 minutes. ¹H NMR (400 MHz, CDCl₃) δ 8.98-8.71 (m, 1H), 8.62 (s, 1H), 8.08 (d, J 7.8 Hz, 1H), 7.88 (d, J 7.6 Hz, 1H), 7.64 (t, J 7.8 Hz, 1H), 7.25-7.17 (m, 1H), 7.06 (d, J 7.6 Hz, 2H), 6.24 (s, 1H), 5.25 (d, J 7.1 Hz, 1H), 4.09-4.01 (m, 2H), 4.00-3.94 (m, 1H), 3.92 (s, 4H), 3.03-2.97 (m, 1H), 2.76-2.65 (m, 1H), 2.62-2.49 (m, 2H), 2.17-2.07 (m, 2H), 2.00 (s, 6H), 1.71-1.60 (m, 1H), 1.45 (d, J 14.9 Hz, 1H), 0.56 (s, 9H). LC method X.

### Step 6: (11R)-6-(2,6-Dimethylphenyl)-11-(2,2-dimethylpropyl)-2,2-dioxo-12-[(3-oxocyclobutyl)methyl]-9-oxa-2λ⁶-thia-3,5,12,19-tetrazatricyclo[12.3.1.14,8]nonadeca-1(18),4(19),5,7,14,16-hexaen-13-one

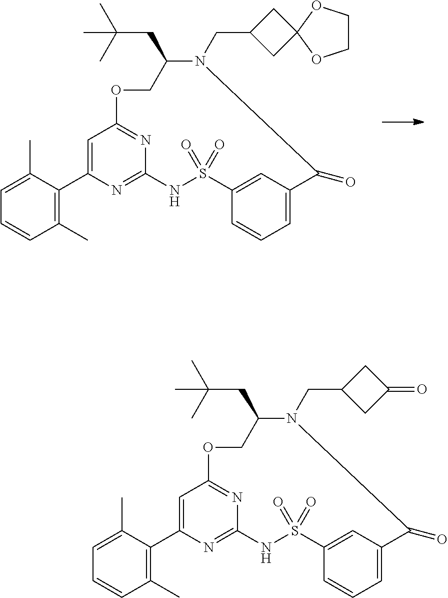 [SMILES: Cc1cccc(C)c1-c1cc2nc(n1)NS(=O)(=O)c1cccc(c1)C(=O)N(CC1CC3(C1)OCCO3)[C@H](CC(C)(C)C)CO2 ; Cc1cccc(C)c1-c1cc2nc(n1)NS(=O)(=O)c1cccc(c1)C(=O)N(CC1CC(=O)C1)[C@H](CC(C)(C)C)CO2]

To a stirred solution of (11R)-6-(2,6-dimethylphenyl)-11-(2,2-dimethylpropyl)-12-(5,8-dioxaspiro[3.4]octan-2-ylmethyl)-2,2-dioxo-9-oxa-2λ⁶-thia-3,5,12,19-tetrazatricyclo[12.3.1.14,8]nonadeca-1(18),4(19),5,7,14,16-hexaen-13-one (7.08 g, 11.405 mmol) in acetone (130 mL) and water (13 mL) was added p-toluenesulfonic acid hydrate (325 mg, 1.7086 mmol). The reaction mixture was stirred in an oil bath at 70° C. for 16 h. The solvent was then evaporated to dryness and the crude residue was diluted in EtOAc (100 mL). Saturated aqueous KHCO₃ (100 mL) was added and the phases were separated. The aqueous phase was washed with EtOAc (2×50 mL). The combined organic layers were washed with brine, dried over magnesium sulfate, filtered and concentrated in vacuo. The residue was purified by chromatography on a 80 g silica gel cartridge using a gradient of 30-100% EtOAc in heptanes. The pure fractions were collected and evaporated to dryness. The resulting solid (6 g, 91%) was triturated in MTBE (10 mL) and pentane (60 mL) at room temperature for 1 h and then filtered on a Buchner funnel. The filter cake was rinsed with pentane and dried in vacuo to afford (11R)-6-(2,6-dimethylphenyl)-11-(2,2-dimethylpropyl)-2,2-dioxo-12-[(3-oxocyclobutyl)methyl]-9-oxa-2λ⁶-thia-3,5,12,19-tetrazatricyclo[12.3.1.14,8]nonadeca-1(18),4(19),5,7,14,16-hexaen-13-one (5.65 g, 83%) as a white solid. ESI-MS m/z calc. 576.2406, found 577.3 (M+1)⁺; Retention time: 4.07 minutes. ¹H NMR (400 MHz, CDCl₃) δ 9.69 (br. s., 1H), 8.63 (s, 1H), 8.07 (d, J 7.8 Hz, 1H), 7.86 (d, J 7.6 Hz, 1H), 7.64 (t, J 7.8 Hz, 1H), 7.25-7.19 (m, 1H), 7.05 (d, J 7.6 Hz, 2H), 6.21 (s, 1H), 5.29 (dd, J=10.1, 3.8 Hz, 1H), 4.16 (dd, J=13.6, 6.0 Hz, 1H), 4.13-4.03 (m, 1H), 4.03-3.93 (m, 1H), 3.36-3.23 (m, 2H), 3.13-3.03 (m, 2H), 3.02-2.83 (m, 2H), 1.98 (s, 6H), 1.64 (dd, J=15.2, 8.6 Hz, 1H), 1.48 (d, J=14.7 Hz, 1H), 0.58 (s, 9H). LC method Y.

### Step 7: (11R)-12-({3-[(2R,6S)-2,6-dimethylmorpholin-4-yl]cyclobutyl}methyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione, diastereomer 1 (Compound 63), and (11R)-12-({3-[(2R,6S)-2,6-dimethylmorpholin-4-yl]cyclobutyl}methyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione, diastereomer 2 (Compound 64)

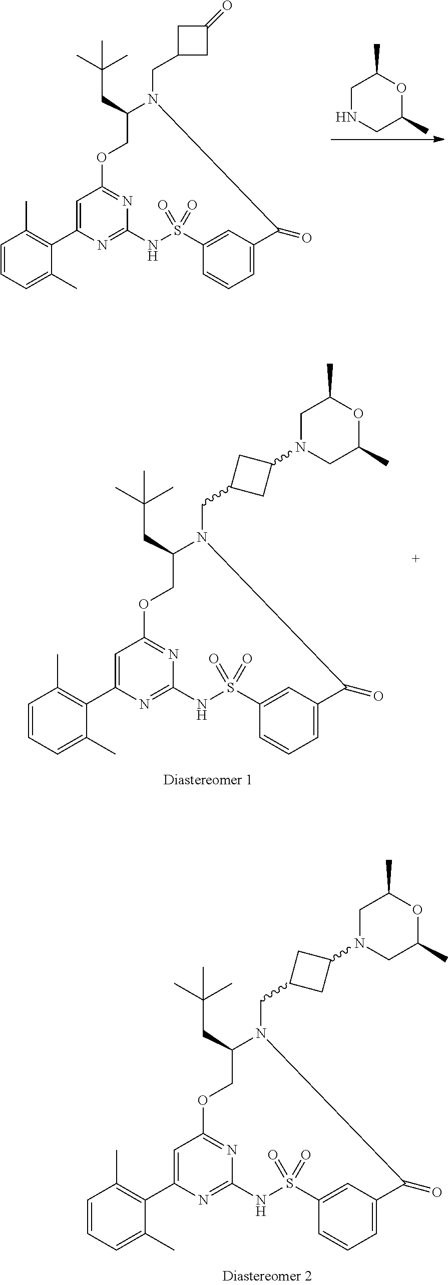

(11R)-6-(2,6-dimethylphenyl)-11-(2,2-dimethylpropyl)-12-[(3-oxocyclobutyl)methyl]-9-oxa-2λ⁶-thia-3,5,12,19-tetraazatricyclo[12.3.1.14,8]nonadeca-1(18),4,6,8(19),14,16-hexaene-2,2,13-trione (50 mg, 0.08670 mmol) and (2S,6R)-2,6-dimethylmorpholine (20 mg, 0.1737 mmol) were combined and stirred in dichloromethane (0.5 mL) for 15 minutes. Sodium triacetoxyborohydride (55 mg, 0.2595 mmol) was added. The reaction mixture was stirred at room temperature for 1 hour. The reaction mixture was diluted with methanol and filtered before purification by UV-triggered reverse-phase HPLC using a 10-50% acetonitrile/water gradient over 60 minutes with 5 mM HCl acid modifier. Both desired stereoisomer products were isolated separately. Diastereomer 1, (11R)-12-({3-[(2R,6S)-2,6-dimethylmorpholin-4-yl]cyclobutyl}methyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (17.0 mg, 58%) was obtained as a white solid. ESI-MS m/z calc. 675.34546, found 676.3 (M+1)⁺; Retention time: 1.38 minutes (LC method A), and diastereomer 2 (11R)-12-({3-[(2R,6S)-2,6-dimethylmorpholin-4-yl]cyclobutyl}methyl)-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (13.4 mg, 46%) was obtained as a white solid. ESI-MS m/z calc. 675.34546, found 676.4 (M+1)⁺; Retention time: 1.4 minutes (LC method A).

### Example 50: Preparation of Compound 65 and Compound 66

### Step 1: (11R)-12-[[3-(2,6-Dimethylmorpholin-4-yl)cyclobutyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 65), and (11R)-12-[[3-(2,6-dimethylmorpholin-4-yl)cyclobutyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 66)

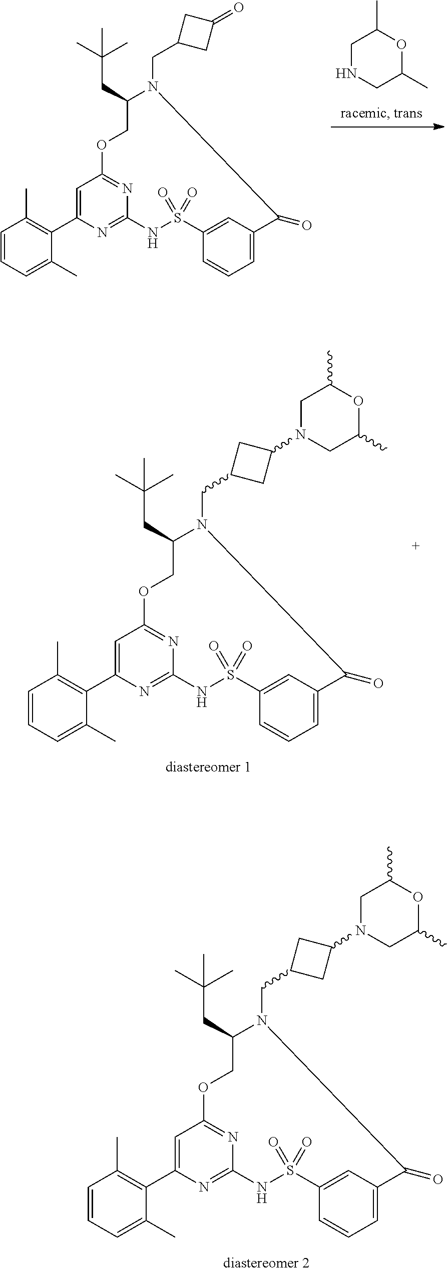

(11R)-6-(2,6-dimethylphenyl)-11-(2,2-dimethylpropyl)-12-[(3-oxocyclobutyl)methyl]-9-oxa-2λ⁶-thia-3,5,12,19-tetraazatricyclo[12.3.1.14,8]nonadeca-1(18),4,6,8(19),14,16-hexaene-2,2,13-trione (100 mg, 0.1734 mmol) and trans-2,6-dimethylmorpholine (racemic, 40 mg, 0.3473 mmol) were combined and dissolved in dichloromethane (1.0 mL). After stirring at room temperature for 15 minutes, sodium triacetoxyborohydride (110 mg, 0.5190 mmol) was added. After 1 hour of stirring at room temperature, the reaction mixture was diluted with DMSO (3 mL) and methanol (1 mL) and filtered. The products were isolated by UV-triggered reverse-phase HPLC using a 20-30% acetonitrile/water gradient over 30 minutes with 5 mM HCl acid modifier to give two isomers: Diastereomer 1 (11R)-12-[[3-(2,6-dimethylmorpholin-4-yl)cyclobutyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (36.1 mg, 62%) was obtained as a white solid. ESI-MS m/z calc. 675.34546, found 676.3 (M+1)⁺; Retention time: 1.37 minutes (LC method A); and diastereomer 2 (11R)-12-[[3-(2,6-dimethylmorpholin-4-yl)cyclobutyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (27.9 mg, 46%) was obtained as a white solid. ESI-MS m/z calc. 675.34546, found 676.3 (M+1)⁺; Retention time: 1.4 minutes (LC method A).

### Example 51: Preparation of Compound 67 and Compound 68

### Step 1: (11R)-12-[(3-benzyl-3-hydroxycyclobutyl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 1 (Compound 67), and (11R)-12-[(3-benzyl-3-hydroxycyclobutyl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione, diastereomer 2 (Compound 68)

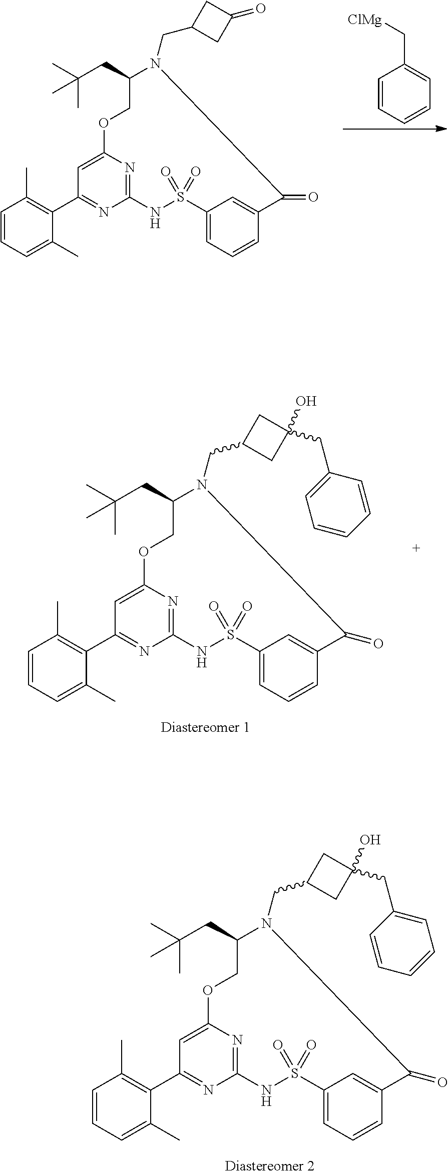

A 4 mL vial was charged under nitrogen with (11R)-6-(2,6-dimethylphenyl)-11-(2,2-dimethylpropyl)-12-[(3-oxocyclobutyl)methyl]-9-oxa-2λ⁶-thia-3,5,12,19-tetraazatricyclo[12.3.1.14,8]nonadeca-1(18),4,6,8(19),14,16-hexaene-2,2,13-trione (57 mg, 0.09884 mmol) and anhydrous THE (0.85 mL). The mixture was cooled down in ice. benzyl(chloro)magnesium (0.075 mL of 2 M, 0.1500 mmol) (2 M in THF) was added. The mixture was stirred in the ice bath for 5-10 min, then at room temperature for 22 h. The mixture was cooled down in ice and quenched by adding an aqueous saturated solution of ammonium chloride (5 drops) and DMSO (2 mL). The solution was microfiltered through a syringe filter disc and purified by reverse phase preparative HPLC (C₁₈) using a gradient of acetonitrile in water (0-40% over 00 min, 40-80% over 20 min, 80-100% over 5 min) and HCl as a modifier, which resulted after evaporation in the isolation of two separated isomers: Diastereomer 1, more polar isomer, peak 1. (11R)-12-[(3-benzyl-3-hydroxycyclobutyl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (6.1 mg, 18%). ESI-MS m/z calc. 668.3032, found 669.3 (M+1)⁺; Retention time: 1.87 minutes (LC method A); and diastereomer 2, less polar isomer peak 2. (11R)-12-[(3-benzyl-3-hydroxycyclobutyl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (6.8 mg, 20%). ESI-MS m/z calc. 668.3032, found 669.35 (M+1)⁺; Retention time: 1.95 minutes (LC method A).

### Example 52: Preparation of Compound 69

### Step 1: 3-[[4-(2,6-Dimethylphenyl)-6-[(2R)-2-[(3-methoxycarbonyl-1-bicyclo[1.1.1]pentanyl)methylamino]-4,4-dimethyl-pentoxy]pyrimidin-2-yl]sulfamoyl]benzoic acid

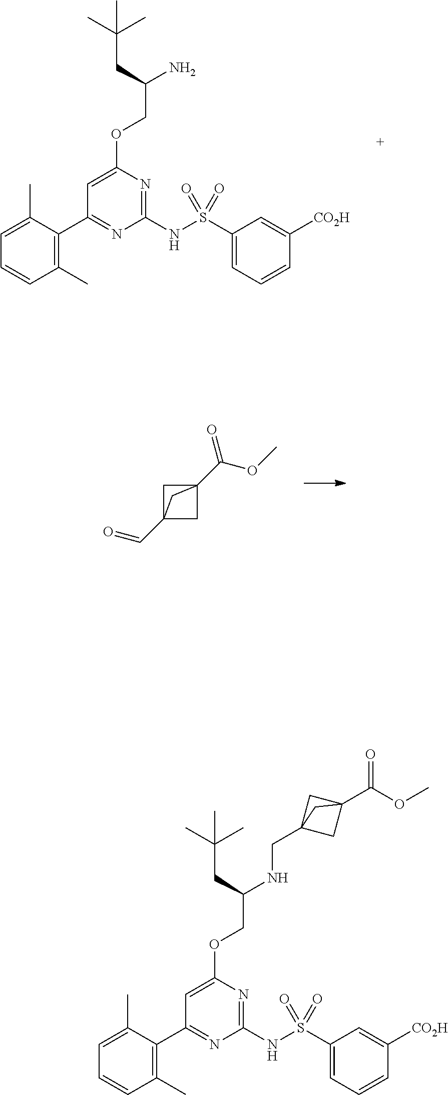 [SMILES: Cc1cccc(C)c1-c1cc(OC[C@H](N)CC(C)(C)C)nc(NS(=O)(=O)c2cccc(C(=O)O)c2)n1 ; COC(=O)C12CC(C=O)(C1)C2 ; COC(=O)C12CC(CN[C@@H](COc3cc(-c4c(C)cccc4C)nc(NS(=O)(=O)c4cccc(C(=O)O)c4)n3)CC(C)(C)C)(C1)C2]

In a 4 mL vial, to a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (200 mg, 0.3642 mmol) and methyl 1-formylbicyclo[1.1.1]pentane-3-carboxylate (57 mg, 0.3697 mmol) in anhydrous dichloromethane (0.6 mL) were added glacial acetic acid (30 μL, 0.5275 mmol), DIEA (350 μL, 2.009 mmol) and sodium triacetoxyborohydride (400 mg, 1.887 mmol), in that order. The vial was purged with nitrogen briefly and capped and allowed to stir at ambient temperature for 5 h. Then methanol (0.3 mL) and water (0.2 mL) were added in that order, and the mixture was concentrated under reduced pressure. The residue was taken up in DMSO (2 mL), micro-filtered, and purified by reverse-phase HPLC (Cis column, 1-99% acetonitrile in water over 15 min, HCl as modifier) to give 3-[[4-(2,6-dimethylphenyl)-6-[(2R)-2-[(3-methoxycarbonyl-1-bicyclo[1.1.1]pentanyl)methylamino]-4,4-dimethyl-pentoxy]pyrimidin-2-yl]sulfamoyl]benzoic acid (hydrochloride salt) (129 mg, 52%) as white solid. ¹H NMR (400 MHz, DMSO-d₆) δ 13.39 (s, 1H), 8.83 (s, 1H), 8.65 (s, 1H), 8.45 (t, J 1.8 Hz, 1H), 8.13 (t, J 9.3 Hz, 2H), 7.70 (t, J 7.8 Hz, 1H), 7.28-7.23 (m, 1H), 7.14 (d, J 7.6 Hz, 2H), 6.35 (s, 1H), 4.42 (d, J=12.4 Hz, 1H), 4.28-4.14 (m, 1H), 3.59 (s, 2H), 3.49 (s, 1H), 3.40 (s, 1H), 3.15 (s, 2H), 2.05 (s, 6H), 2.00 (s, 6H), 1.70-1.54 (m, 2H), 0.92 (s, 9H). ESI-MS m/z calc. 650.2774, found 651.3 (M+1)⁺; Retention time: 1.23 minutes (LC method A).

### Step 2: Methyl 3-[[(11R)-6-(2,6-dimethylphenyl)-11-(2,2-dimethylpropyl)-2,2,13-trioxo-9-oxa-2)⁶-thia-3,5,12,19-tetrazatricyclo[12.3.1.14,8]nonadeca-1(18),4(19),5,7,14,16-hexaen-12-yl]methyl]bicyclo[1.1.1]pentane-1-carboxylate

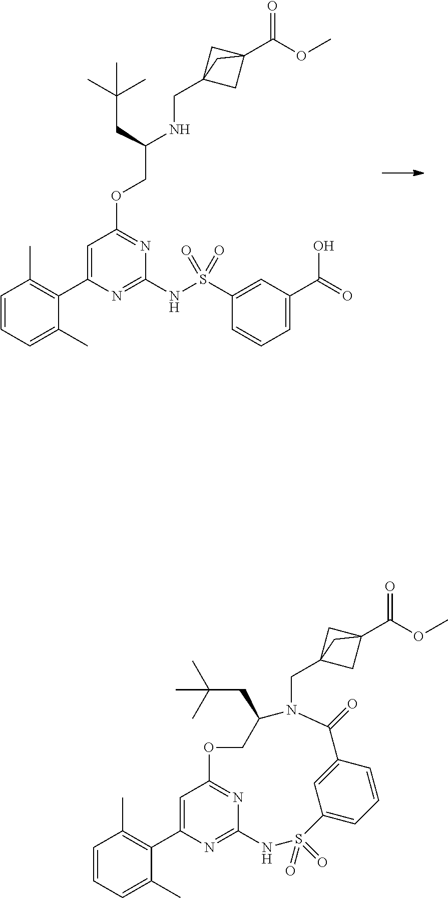 [SMILES: COC(=O)C12CC(CN[C@@H](COc3cc(-c4c(C)cccc4C)nc(NS(=O)(=O)c4cccc(C(=O)O)c4)n3)CC(C)(C)C)(C1)C2 ; COC(=O)C12CC(CN3C(=O)c4cccc(c4)S(=O)(=O)Nc4nc(cc(-c5c(C)cccc5C)n4)OC[C@H]3CC(C)(C)C)(C1)C2]

In a 25 mL flask, to a stirred solution of 3-[[4-(2,6-dimethylphenyl)-6-[(2R)-2-[(3-methoxycarbonyl-1-bicyclo[1.1.1]pentanyl)methylamino]-4,4-dimethyl-pentoxy]pyrimidin-2-yl]sulfamoyl]benzoic acid (hydrochloride salt) (120 mg, 0.1746 mmol) in anhydrous DMF (6 mL) were added 4-methylmorpholine (100 μL, 0.9096 mmol) and 2-chloro-4,6-dimethoxy-1,3,5-triazine (50 mg, 0.2848 mmol) (CDMT), in that order, at 0-5° C. (ice-water bath) under nitrogen. The bath was removed after 15 min and the reaction was allowed to warm to room temperature and stirring continued at that temperature over the weekend (60 h). Then most of the solvent was removed under reduced pressure (below 40° C. bath temperature). The residue was taken up in DMSO (2 mL), micro-filtered, and purified by reverse-phase HPLC (1-99% acetonitrile in water over 15 min, HCl as modifier) to give methyl 3-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]bicyclo[1.1.1]pentane-1-carboxylate (30 mg, 27%) as white solid. ¹H NMR (400 MHz, DMSO-d₆) δ 13.1 (s, 1H), 8.48 (s, 1H), 7.96 (s, 1H), 7.69 (s, 2H), 7.25 (t, J 7.8 Hz, 1H), 7.12 (d, J 7.5 Hz, 2H), 6.43 (s, 1H), 5.17-4.97 (m, 1H), 4.16 (t, J 11.0 Hz, 1H), 3.93-3.77 (m, 2H), 3.59 (s, 3H), 3.26 (d, J 14.5 Hz, 1H), 2.17-1.82 (m, 12H), 1.60 (dd, J 15.3, 8.8 Hz, 1H), 1.31 (d, J 15.0 Hz, 1H), 0.49 (s, 9H). ESI-MS m/z calc. 632.26685, found 633.3 (M+1)⁺; Retention time: 1.81 minutes (LC method A).

### Step 3: (11R)-6-(2,6-Dimethylphenyl)-11-(2,2-dimethylpropyl)-12-[[3-(1-hydroxy-1-methyl-ethyl)-1-bicyclo[1.1.1]pentanyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 69)

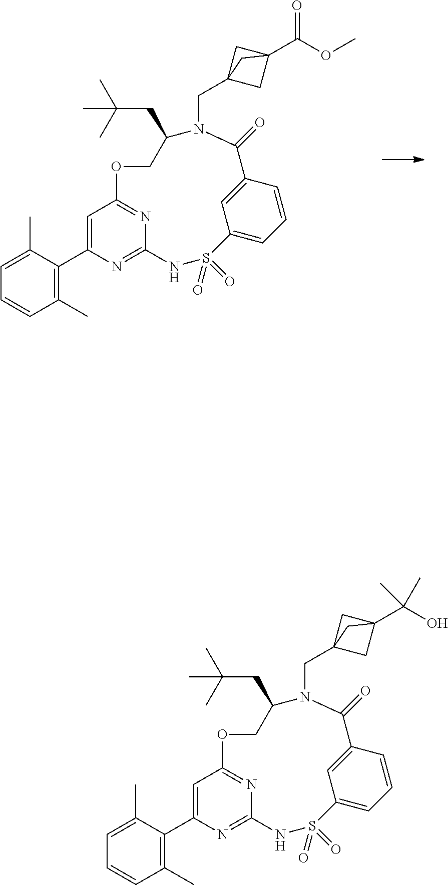 [SMILES: COC(=O)C12CC(CN3C(=O)c4cccc(c4)S(=O)(=O)Nc4nc(cc(-c5c(C)cccc5C)n4)OC[C@H]3CC(C)(C)C)(C1)C2 ; Cc1cccc(C)c1-c1cc2nc(n1)NS(=O)(=O)c1cccc(c1)C(=O)N(CC13CC(C(C)(C)O)(C1)C3)[C@H](CC(C)(C)C)CO2]

To a stirred solution of methyl 3-{[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4,6,8(19),14(18),15-hexaen-12-yl]methyl}bicyclo[1.1.1]pentane-1-carboxylate (25 mg, 0.03951 mmol) in anhydrous tetrahydofuran (1 mL) was added Methyl Magnesium Bromide (100 μL of 3 M, 0.3000 mmol) (3M in diethyl ether) at 0° C. under nitrogen. Stirring continued for 1 h at that temperature. Then the reaction was quenched with glacial acetic acid (20 μL, 0.3517 mmol). The volatiles were removed, and the residue was taken up in DMSO (1 mL), microfiltered through a syringe filter disc and purified from preparative reverse phase HPLC (C₁₈) using 1-99% acetonitrile in water over 15 min and HCl as a modifier to afford (11R)-6-(2,6-dimethylphenyl)-11-(2,2-dimethylpropyl)-12-[[3-(1-hydroxy-1-methyl-ethyl)-1-bicyclo[1.1.1]pentanyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (15 mg, 59%) as white solid. ¹H NMR (400 MHz, DMSO-d₆) δ 8.49 (s, 1H), 7.87 (d, J 6.6 Hz, 1H), 7.57 (s, 2H), 7.17 (s, 1H), 7.06 (d, J 7.0 Hz, 2H), 6.03 (s, 1H), 5.06 (d, J 6.4 Hz, 1H), 3.96 (s, 2H), 3.83 (d, J 14.4 Hz, 1H), 3.59 (s, 3H), 3.31 (s, 3H), 3.20 (d, J 14.4 Hz, 1H), 2.12-1.87 (m, 12H), 1.62-1.46 (m, 1H), 1.32 (d, J 14.9 Hz, 1H), 0.48 (s, 9H). ESI-MS m/z calc. 632.3032, found 633.3 (M+1)⁺; Retention time: 1.81 minutes (LC method A).

### Example 53a: Preparation of Compound 70

### Step 1: Methyl 5-[(4-methoxyphenyl)methylsulfanyl]-1-methyl-pyrazole-3-carboxylate

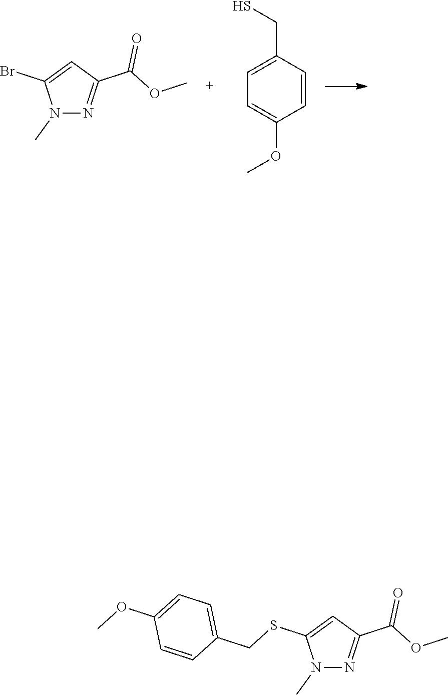

To a sealed tube was added methyl 5-bromo-1-methyl-pyrazole-3-carboxylate (4.71 g, 21.503 mmol), (4-methoxyphenyl)methanethiol (3.32 g, 21.526 mmol) and diisopropylethylamine (5.5650 g, 7.5 mL, 43.058 mmol) in dioxane (100 mL). The mixture was sparged with nitrogen gas for 15 minutes, then added Xantphos (1.24 g, 2.1430 mmol) and Pd₂dba₃ (980 mg, 1.0702 mmol). The tube was capped and heated in an oil bath set at 100° C. for 5 hours. Once cooled to room temperature, the reaction mixture was transferred to a 1.0-L separatory funnel with water (350 mL) and the aqueous layer was extracted with ethyl acetate (1×300 mL, 1×200 mL). The combined organic layers were washed with brine, dried over sodium sulfate, filtered and concentrated under reduced pressure. The residue was purified by silica gel chromatography on a 220-g column, eluting from 0% to 40% ethyl acetate in heptanes, to afford methyl 5-[(4-methoxyphenyl)methylsulfanyl]-1-methyl-pyrazole-3-carboxylate (5.2 g, 83%) as a pale-yellow solid. ESI-MS m/z calc. 292.0882, found 293.1 (M+1)⁺; Retention time: 1.94 minutes, LC method K.

### Step 2: Methyl 5-chlorosulfonyl-1-methyl-pyrazole-3-carboxylate

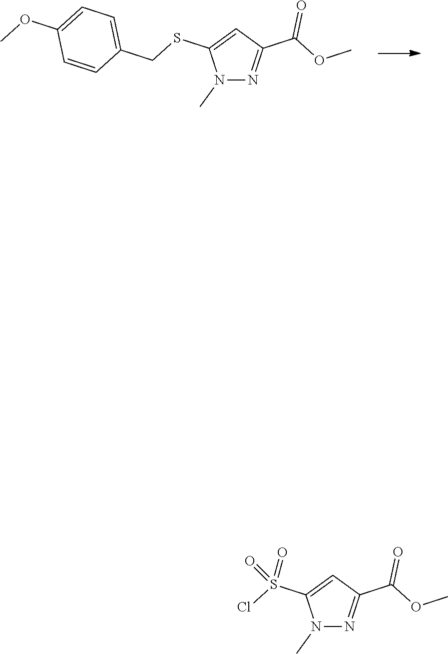

A solution of methyl 5-[(4-methoxyphenyl)methylsulfanyl]-1-methyl-pyrazole-3-carboxylate (4.74 g, 16.213 mmol) in acetic acid (50 mL) and water (25 mL) was treated with N-chlorosuccinimide (6.6 g, 49.426 mmol) at room temperature for 1.5 hours. The reaction was then quenched by adding to a 2.0-L separatory funnel containing cold water (1.5 L) and the aqueous layer was extracted with MTBE (3×250 mL). The combined organic layers were washed with cold water (300 mL), brine, dried over sodium sulfate, filtered and concentrated under reduced pressure. The residue was purified by silica gel chromatography on a 220-g column eluting from 0% to 40% ethyl acetate in heptanes to afford methyl 5-chlorosulfonyl-1-methyl-pyrazole-3-carboxylate (3.62 g, 90%) as a colorless oil. ¹H NMR. ¹H NMR (300 MHz, CDCl₃) δ 7.50 (s, 1H), 4.30 (s, 3H), 3.96 (s, 3H). ESI-MS m/z calc. 237.9815, found 239.0 (M+1)⁺; Retention time: 1.81 minutes, LC method K.

### Step 3: Methyl 5-[[4-chloro-6-(2,6-dimethylphenyl)pyrimidin-2-yl]sulfamoyl]-1-methyl-pyrazole-3-carboxylate

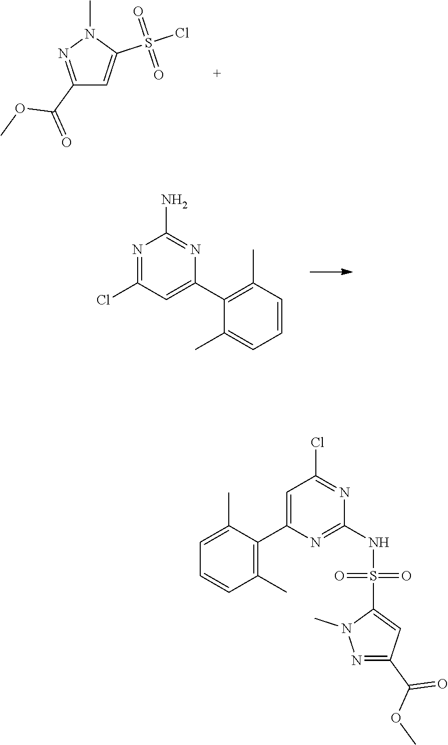 [SMILES: COC(=O)c1cc(S(=O)(=O)Cl)n(C)n1 ; Cc1cccc(C)c1-c1cc(Cl)nc(N)n1 ; COC(=O)c1cc(S(=O)(=O)Nc2nc(Cl)cc(-c3c(C)cccc3C)n2)n(C)n1]

4-chloro-6-(2,6-dimethylphenyl)pyrimidin-2-amine (7.65 g, 32.735 mmol) was dissolved in THE (140 mL) and cooled in an ice bath under stirring and nitrogen. To the cold solution, methyl 5-chlorosulfonyl-1-methyl-pyrazole-3-carboxylate (6.24 g, 26.147 mmol) in solution in THE (45 mL) was added. At 0° C., sodium tert-butoxide (18.5 mL of 40% w/v, 66.584 mmol) was added dropwise (the color was colorless before and yellow after the addition) and the reaction was stirred at room temperature for two hour. The reaction was quenched with HCl 1 N (50 mL). The reaction was diluted with water (150 mL) and EtOAc (250 mL). The organic phase was isolated, and the aqueous phase was extracted with EtOAc (200 mL). The organic phases were combined and washed with water (100 mL and brine (100 mL). The organic phase was dried over sodium sulfate, filtered, and concentrated. The crude was purified by chromatography on silica gel, 120 g, eluted with EtOAc-heptane 5% to 35% to give methyl 5-[[4-chloro-6-(2,6-dimethylphenyl)pyrimidin-2-yl]sulfamoyl]-1-methyl-pyrazole-3-carboxylate (9.15 g, 80%) as a beige solid. ESI-MS m/z calc. 435.0768, found 436.1 (M+1)⁺; Retention time: 1.98 minutes, LC method K.

### Step 4: 5-[[4-Chloro-6-(2,6-dimethylphenyl)pyrimidin-2-yl]sulfamoyl]-1-methyl-pyrazole-3-carboxylic acid

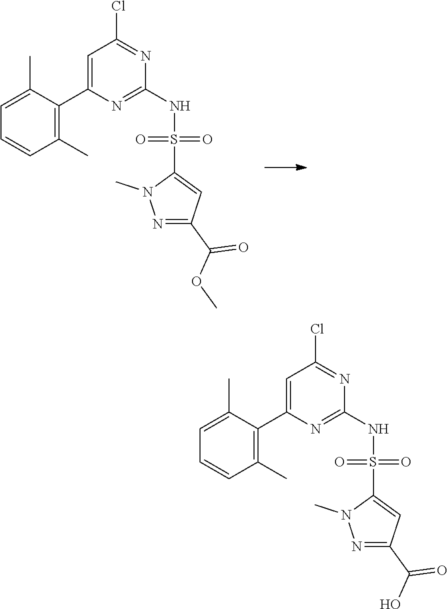 [SMILES: COC(=O)c1cc(S(=O)(=O)Nc2nc(Cl)cc(-c3c(C)cccc3C)n2)n(C)n1 ; Cc1cccc(C)c1-c1cc(Cl)nc(NS(=O)(=O)c2cc(C(=O)O)nn2C)n1]

A mixture of methyl 5-[[4-chloro-6-(2,6-dimethylphenyl)pyrimidin-2-yl]sulfamoyl]-1-methyl-pyrazole-3-carboxylate (832 mg, 1.9088 mmol) in THE (25 mL) and water (25 mL) was treated with lithium hydroxide hydrate (240 mg, 5.7192 mmol) and stirred vigorously at room temperature for 2.5 hours. Most of the THF was removed under reduced pressure, and the remaining aqueous layer was transferred to a 250-mL separatory funnel with water (100 mL) and the aqueous layer was washed with DCM (50 mL). The aqueous layer was acidified to a pH of about 4 using solid citric acid and extracted with ethyl acetate (3×50 mL). The combined organic layers were washed with water (50 mL), brine (50 mL), dried over sodium sulfate, filtered and concentrated under reduced pressure to afford 5-[[4-chloro-6-(2,6-dimethylphenyl)pyrimidin-2-yl]sulfamoyl]-1-methyl-pyrazole-3-carboxylic acid (719 mg, 86%) as a white solid. ¹H NMR (300 MHz, DMSO-d₆) δ 13.14 (br. s., 2H), 7.37 (s, 1H), 7.31-7.22 (m, 1H), 7.18-7.08 (m, 3H), 3.99 (s, 3H), 1.93 (s, 6H). ESI-MS m/z calc. 421.0612, found 422.1 (M+1)⁺; Retention time: 2.62 minutes, LC method U.

### Step 5: 5-[[4-[(2R)-2-Amino-4,4-dimethyl-pentoxy]-6-(2,6-dimethylphenyl)pyrimidin-2-yl]sulfamoyl]-1-methyl-pyrazole-3-carboxylic acid

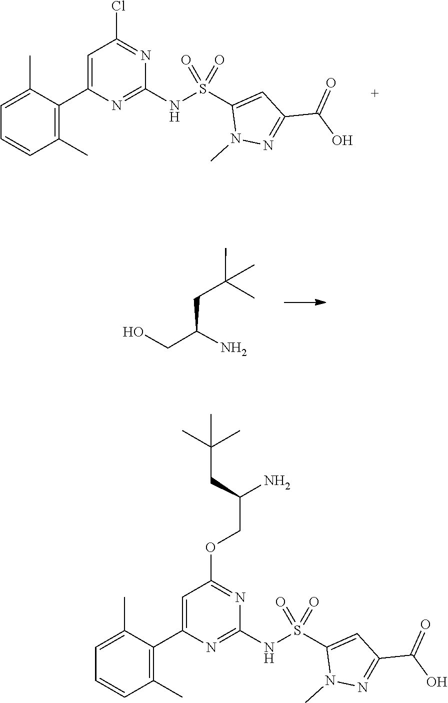

5-[[4-Chloro-6-(2,6-dimethylphenyl)pyrimidin-2-yl]sulfamoyl]-1-methyl-pyrazole-3-carboxylic acid (1.50 g, 3.556 mmol) and (2R)-2-amino-4,4-dimethyl-pentan-1-ol (hydrochloride salt) (656 mg, 3.912 mmol) were combined and dissolved/suspended in THE (12 mL). Solid sodium tert-butoxide (1.71 g, 17.79 mmol) was added in gradual portions over 2 minutes. The reaction mixture was allowed to stir at room temperature for 2 hours. The reaction was quenched with the addition of aqueous HCl (75 mL, 1 M). It was then extracted with EtOAc (3×75 mL). The organic layers were combined, washed with brine (1×100 mL), dried over sodium sulfate, filtered and concentrated under reduced pressure. The crude product was chromatographed on a 24-gram silica gel column eluting with a 0-20% MeOH/DCM gradient over 40 minutes; product eluted at 10% MeOH. The obtained white solid was dissolved into MeOH/DCM, and HCl (800 μL of 4 M, 3.200 mmol) in dioxane was added. After brief stirring, volatiles were removed under reduced pressure to provide 5-[[4-[(2R)-2-amino-4,4-dimethyl-pentoxy]-6-(2,6-dimethylphenyl)pyrimidin-2-yl]sulfamoyl]-1-methyl-pyrazole-3-carboxylic acid (hydrochloride salt) (1.112 g, 57%) was obtained as a pinkish-white solid. ¹H NMR (400 MHz, DMSO-d₆) δ 8.15 (s, 2H), 7.32 (t, J 7.6 Hz, 1H), 7.19 (s, 1H), 7.17 (s, 1H), 7.12 (s, 1H), 6.33 (s, 1H), 4.31 (dd, J=11.9, 3.1 Hz, 1H), 4.13 (d, J=4.1 Hz, 1H), 4.03 (s, 3H), 3.57 (s, 1H), 2.13 (s, 6H), 1.63-1.47 (m, 2H), 0.95 (s, 9H). ESI-MS m/z calc. 516.2155, found 517.2 (M+1)⁺; Retention time: 1.16 minutes (LC method A).

### Step 6: 5-[[4-(2,6-Dimethylphenyl)-6-[(2R)-4,4-dimethyl-2-(pyrimidin-2-ylmethylamino)pentoxy]pyrimidin-2-yl]sulfamoyl]-1-methyl-pyrazole-3-carboxylic acid

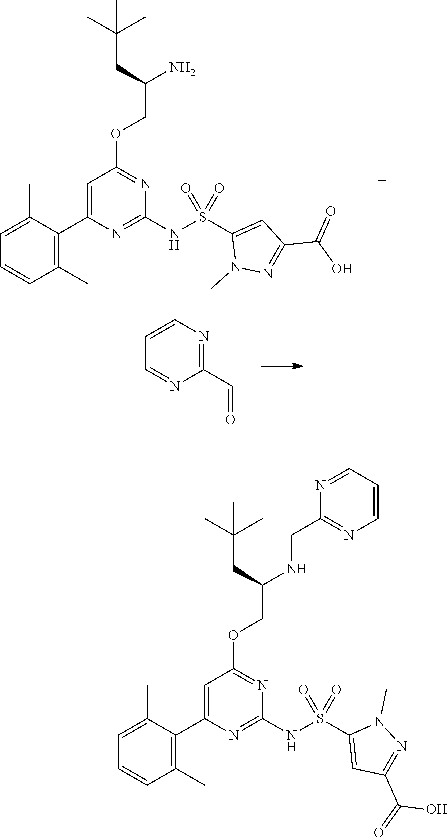 [SMILES: Cc1cccc(C)c1-c1cc(OC[C@H](N)CC(C)(C)C)nc(NS(=O)(=O)c2cc(C(=O)O)nn2C)n1 ; O=Cc1ncccn1 ; Cc1cccc(C)c1-c1cc(OC[C@@H](CC(C)(C)C)NCc2ncccn2)nc(NS(=O)(=O)c2cc(C(=O)O)nn2C)n1]

5-[[4-[(2R)-2-amino-4,4-dimethyl-pentoxy]-6-(2,6-dimethylphenyl)pyrimidin-2-yl]sulfamoyl]-1-methyl-pyrazole-3-carboxylic acid (hydrochloride salt) (75 mg, 0.1356 mmol) and pyrimidine-2-carbaldehyde (approximately 16.13 mg, 0.1492 mmol) were combined in dichloromethane (0.5 mL). Sodium triacetoxyborohydride (approximately 115.0 mg, 0.5424 mmol) was added. The reaction mixture was allowed to stir overnight at room temperature. The product was isolated by mass-triggered reverse-phase HPLC eluting with a 10-99% acetonitrile/water gradient with 5 mM HCl acid modifier. 5-[[4-(2,6-dimethylphenyl)-6-[(2R)-4,4-dimethyl-2-(pyrimidin-2-ylmethylamino)pentoxy]pyrimidin-2-yl]sulfamoyl]-1-methyl-pyrazole-3-carboxylic acid (hydrochloride salt) (14.3 mg, 17%) was obtained. ESI-MS m/z calc. 608.2529, found 609.3 (M+1)⁺; Retention time: 1.02 minutes; LC method A.

### Step 7: (10R)-15-(2,6-Dimethylphenyl)-10-(2,2-dimethylpropyl)-5-methyl-3,3-dioxo-9-(pyrimidin-2-ylmethyl)-12-oxa-3λ⁶-thia-2,5,6,9,16,17-hexazatricyclo[11.3.1.14,7]octadeca-1(17),4(18),6,13,15-pentaen-8-one (Compound

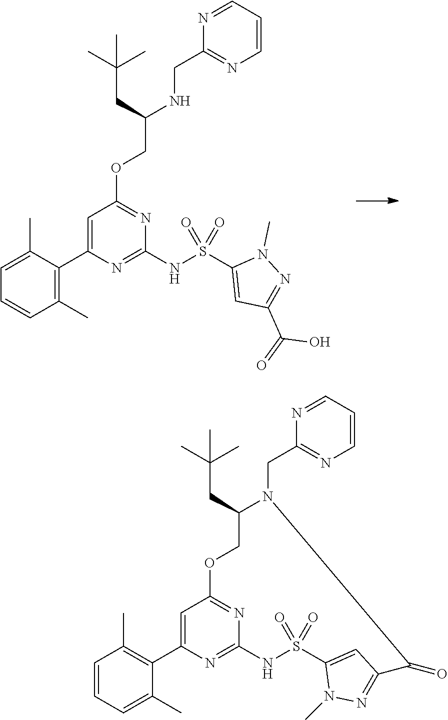 [SMILES: Cc1cccc(C)c1-c1cc(OC[C@@H](CC(C)(C)C)NCc2ncccn2)nc(NS(=O)(=O)c2cc(C(=O)O)nn2C)n1 ; Cc1cccc(C)c1-c1cc2nc(n1)NS(=O)(=O)c1cc(nn1C)C(=O)N(Cc1ncccn1)[C@H](CC(C)(C)C)CO2]

5-[[4-(2,6-dimethylphenyl)-6-[(2R)-4,4-dimethyl-2-(pyrimidin-2-ylmethylamino)pentoxy]pyrimidin-2-yl]sulfamoyl]-1-methyl-pyrazole-3-carboxylic acid (24.35 mg, 0.04 mmol) was dissolved in and DMF (1 mL). To this solution was added dropwise a solution of CDMT (1.3 eq) in DMF (0.1 mL). After stirring at room temperature for 30 minutes, a solution of 4-methylmorpholine (approximately 6.069 mg, 6.597 μL, 0.06000 mmol) in DMF (0.1 mL) was added last. The reaction mixture was allowed to stir overnight at room temperature. The product was isolated by mass-triggered reverse-phase HPLC: Samples were purified using a reverse phase HPLC-MS method using a Luna C₁₈ (2) column (75×30 mm, 5 μm particle size) sold by Phenomenex (pn: 00C-4252-U0-AX), and a dual gradient run from 1-99% mobile phase B over 15.0 minutes. Mobile phase A=water (5 mM HCl). Mobile phase B=acetonitrile. Flow rate=50 mL/min, injection volume=950 μL, and column temperature=25° C. (10R)-15-(2,6-dimethylphenyl)-10-(2,2-dimethylpropyl)-5-methyl-3,3-dioxo-9-(pyrimidin-2-ylmethyl)-12-oxa-3λ⁶-thia-2,5,6,9,16,17-hexazatricyclo[11.3.1.14,7]octadeca-1(17),4(18),6,13,15-pentaen-8-one (1.6 mg, 6%) was obtained. ESI-MS m/z calc. 590.2424, found 591.13 (M+1)⁺; Retention time: 1.69 minutes (LC method A).

### Example 53b: Preparation of Compound 71

### Step 1: 3-[(4-{[(2R)-2-{[(tert-Butoxy)carbonyl](methyl)amino}-4-methylpentyl]oxy}-6-(2,6-dimethylphenyl)pyrimidin-2-yl)sulfamoyl]benzoic acid

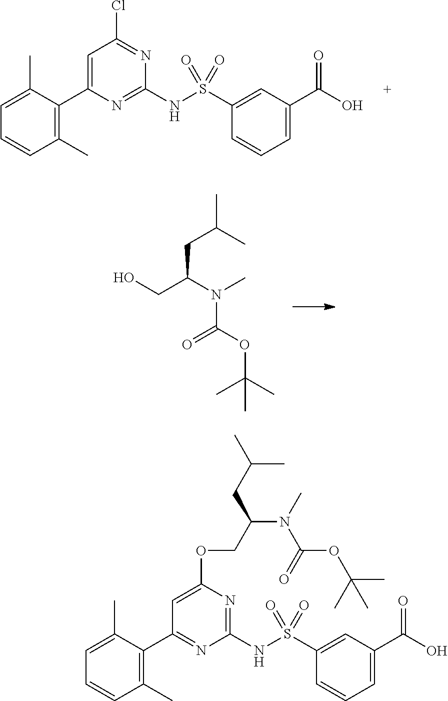

3-[[4-Chloro-6-(2,6-dimethylphenyl)pyrimidin-2-yl]sulfamoyl]benzoic acid (200 mg, 0.4786 mmol) and tert-butyl N-[(2R)-1-hydroxy-4-methylpentan-2-yl]-N-methylcarbamate (approximately 110.7 mg, 0.4786 mmol) were combined and dissolved in THF. Sodium tert-butoxide (approximately 230.0 mg, 2.393 mmol) was added last. The reaction mixture was allowed to stir overnight at room temperature. It was then diluted with EtOAc (7 mL) and washed with HCl (2×7 mL) and brine (1×7 mL). The organic layer was dried over sodium sulfate, filtered and concentrated under reduced pressure. The crude product was purified by UV-triggered reverse-phase HPLC using a Luna C18 (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. 3-[(4-{[(2R)-2-{[(tert-butoxy)carbonyl](methyl)amino}-4-methylpentyl]oxy}-6-(2,6-dimethylphenyl)pyrimidin-2-yl)sulfamoyl]benzoic acid (36 mg, 12%) was obtained. ESI-MS m/z calc. 612.2618, found 613.3 (M+1)⁺; Retention time: 1.97 minutes; LC method A.

### Step 2: (11R)-6-(2,6-Dimethylphenyl)-12-methyl-11-(2-methylpropyl)-9-oxa-2λ⁶-thia-3,5,12,19-tetraazatricyclo[12.3.1.14,8]nonadeca-1(18),4(19),5,7,14,16-hexaene-2,2,13-trione (Compound 71)

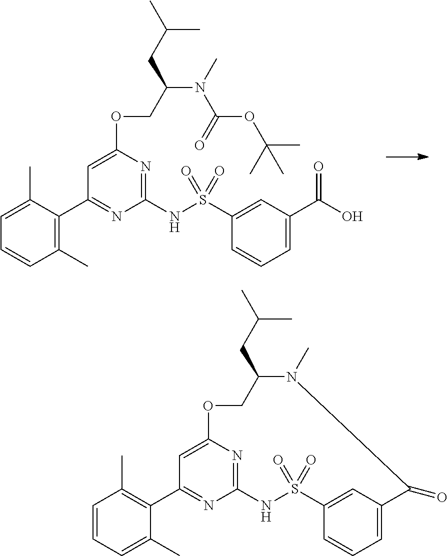

To 3-[(4-{[(2R)-2-{[(tert-butoxy)carbonyl](methyl)amino}-4-methylpentyl]oxy}-6-(2,6-dimethylphenyl)pyrimidin-2-yl)sulfamoyl]benzoic acid (50 mg, 0.08160 mmol) was added a solution of HCl (approximately 204.0 μL of 4 M, 0.8160 mmol) in dioxane. The reaction mixture was allowed to stir at room temperature for 15 minutes. Volatiles were then removed under reduced pressure. The remaining residue was taken up in DMF (0.5 mL), and HATU (approximately 34.13 mg, 0.08976 mmol) was added followed by DIEA (approximately 31.64 mg, 42.64 μL, 0.2448 mmol). After 15 minutes of stirring at room temperature, the product was isolated by UV-triggered reverse-phase HPLC using a Luna C18 (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-6-(2,6-dimethylphenyl)-12-methyl-1l-(2-methylpropyl)-9-oxa-2λ⁶-thia-3,5,12,19-tetraazatricyclo[12.3.1.14,8]nonadeca-1(18),4(19),5,7,14,16-hexaene-2,2,13-trione was obtained. ESI-MS m/z calc. 494.19876, found 495.3 (M+1)⁺; Retention time: 1.64 minutes; LC method A.

### Example 54: Preparation of Compound 72, Compound 73, and Compound 74

### Step 1: 2-(2-Tetrahydropyran-4-ylethylamino)-4-[1-(trifluoromethyl)cyclopropyl]butanenitrile

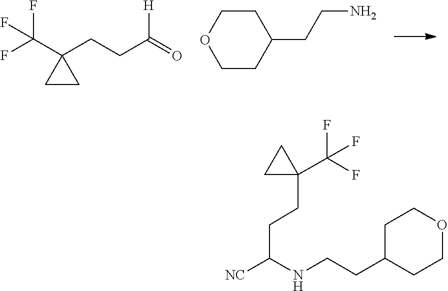

To a stirring solution of 3-[1-(trifluoromethyl)cyclopropyl]propanal (821.9 mg, 4.947 mmol) in acetonitrile (48.19 mL) under nitrogen atmosphere was added 2-tetrahydropyran-4-ylethanamine (639 mg, 4.946 mmol) and trimethylsilylformonitrile (791.4 μL, 5.935 mmol). bromo(dimethyl)sulfonium bromide (109.8 mg, 0.4947 mmol) was then added and the mixture was stirred for 90 min. Diluted with water (48.19 mL) then removed ˜½ of the acetonitrile by rotary evaporation. Extracted the resulting mixture with EtOAc (3×), combined organic phases, dried (sodium sulfate), filtered and concentrated to light tan oil, 2-(2-tetrahydropyran-4-ylethylamino)-4-[1-(trifluoromethyl)cyclopropyl]butanenitrile (1.3 g, 86%) ESI-MS m/z calc. 304.17624, found 305.0 (M+1)⁺; Retention time: 0.39 minutes (LC method D).

### Step 2: 2-(2-Tetrahydropyran-4-ylethylamino)-4-[1-(trifluoromethyl)cyclopropyl]butanoic acid

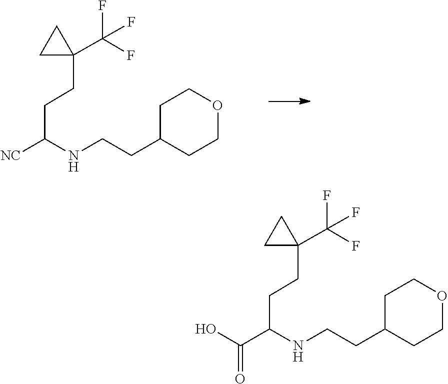 [SMILES: N#CC(CCC1(C(F)(F)F)CC1)NCCC1CCOCC1 ; O=C(O)C(CCC1(C(F)(F)F)CC1)NCCC1CCOCC1]

To a stirring solution 2-(2-tetrahydropyran-4-ylethylamino)-4-[1-(trifluoromethyl)cyclopropyl]butanenitrile (1.3 g, 4.271 mmol) in acetic acid (813.7 μL, 14.31 mmol) in a vial was added HCl (8.123 mL of 37% w/v, 82.43 mmol) and the vial was capped. The mixture was stirred and heated in an aluminum block at 95° C. for 16 h. The mixture was transferred to a round bottom flask using MeOH and was concentrated by rotary evaporation, including treatment with diethyl ether and removing the solvents three times to give 2-(2-tetrahydropyran-4-ylethylamino)-4-[1-(trifluoromethyl)cyclopropyl]butanoic acid as a light tan solid that was dried thoroughly on the high vacuum pump then taken directly to the next step (1.576 g, 100%). ESI-MS m/z calc. 323.17084, found 324.0 (M+1)⁺; Retention time: 0.33 minutes, LC method D.

### Step 3: 2-(2-Tetrahydropyran-4-ylethylamino)-4-[1-(trifluoromethyl)cyclopropyl]butan-1-ol

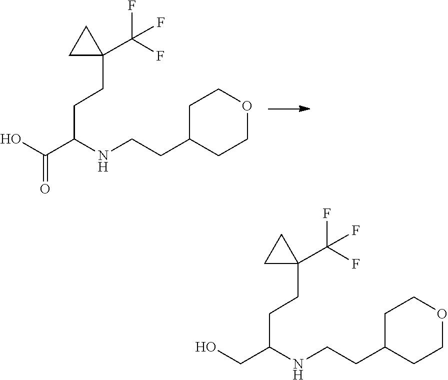 [SMILES: O=C(O)C(CCC1(C(F)(F)F)CC1)NCCC1CCOCC1 ; OCC(CCC1(C(F)(F)F)CC1)NCCC1CCOCC1]

To a stirring solution of 2-(2-tetrahydropyran-4-ylethylamino)-4-[1-(trifluoromethyl)cyclopropyl]butanoic acid (1.576 g, 4.265 mmol) in THE (27.58 mL) under nitrogen atmosphere at 0° C. was slowly added LAH (664.7 mg, 17.06 mmol) and the resulting mixture was stirred at 0° C. for 2 min then allowed to warm to rt and was stirred 75 min. Cooled to 0° C. and quenched by the addition of water (1.279 mL, 71.00 mmol), then KOH (1.279 mL of 15% w/v, 3.419 mmol) then water (2.556 mL, 141.9 mmol). Warmed to rt, added Celite and stirred 5 min then filtered over Celite eluting with ether. The ethereal filtrate was then dried (magnesium sulfate), filtered and concentrated the filtrate by rotary evaporation to an orange oil, 2-(2-tetrahydropyran-4-ylethylamino)-4-[1-(trifluoromethyl)cyclopropyl]butan-1-ol (1.225 g, 93%) ESI-MS m/z calc. 309.19156, found 310.0 (M+1)⁺; Retention time: 0.34 minutes, LC method D.

### Step 4: 3-[[4-(2,6-Dimethylphenyl)-6-[2-(2-tetrahydropyran-4-ylethylamino)-4-[1-(trifluoromethyl)cyclopropyl]butoxy]pyrimidin-2-yl]sulfamoyl]benzoic acid

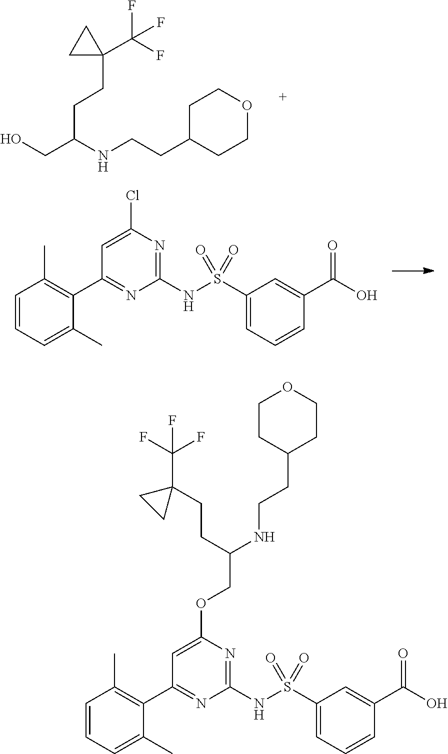

To a stirring solution of 3-[[4-chloro-6-(2,6-dimethylphenyl)pyrimidin-2-yl]sulfamoyl]benzoic acid (675.3 mg, 1.616 mmol) and 2-(2-tetrahydropyran-4-ylethylamino)-4-[1-(trifluoromethyl)cyclopropyl]butan-1-ol (500 mg, 1.616 mmol) in THE (11 mL) at 0° C. was added KOtBu (804.1 μL, 6.464 mmol) and the mixture was stirred at 50° C. for 20 min then removed the acetonitrile by rotary evaporation, dissolved the residue in DMSO, filtered and chromatographed on a 275 g Reverse Phase Column eluting with 20-100% ACN/Water giving 3-[[4-(2,6-dimethylphenyl)-6-[2-(2-tetrahydropyran-4-ylethylamino)-4-[1-(trifluoromethyl)cyclopropyl]butoxy]pyrimidin-2-yl]sulfamoyl]benzoic acid (330 mg, 30%) ESI-MS m/z calc. 690.2699, found 691.1 (M+1)⁺; Retention time: 0.51 minutes, LC method D.

### Step 5: 6-(2,6-dimethylphenyl)-2,2-dioxo-12-(2-tetrahydropyran-4-ylethyl)-11-[2-[1-(trifluoromethyl)cyclopropyl]ethyl]-9-oxa-2λ⁶-thia-3,5,12,19-tetrazatricyclo[12.3.1.14,8]nonadeca-1(18),4(19),5,7,14,16-hexaen-13-one (Compound 74)

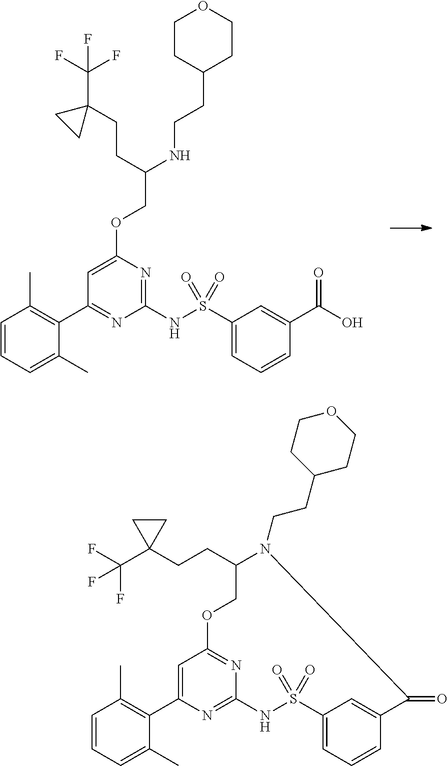 [SMILES: Cc1cccc(C)c1-c1cc(OCC(CCC2(C(F)(F)F)CC2)NCCC2CCOCC2)nc(NS(=O)(=O)c2cccc(C(=O)O)c2)n1 ; Cc1cccc(C)c1-c1cc2nc(n1)NS(=O)(=O)c1cccc(c1)C(=O)N(CCC1CCOCC1)C(CCC1(C(F)(F)F)CC1)CO2]

3-[[4-(2,6-dimethylphenyl)-6-[2-(2-tetrahydropyran-4-ylethylamino)-4-[1-(trifluoromethyl)cyclopropyl]butoxy]pyrimidin-2-yl]sulfamoyl]benzoic acid (325 mg, 0.4705 mmol) was combined with HATU (232.5 mg, 0.6115 mmol) in DMF (19.5 mL), and DIPEA (246 μL, 1.412 mmol) was added. Stirred at rt overnight then diluted with EtOAc and washed with saturated aqueous ammonium chloride (2×), saturated aqueous sodium bicarbonate (2×) and brine (1×), then dried (magnesium sulfate), filtered and conc to an orange oil which was chromatographed on a 275 g Reverse Phase Column eluting with 20-100% ACN/Water giving the intended lactam product still contaminated with the lactone side product. Fractions containing product were concentrated, filtered and purified using a reverse phase HPLC-MS method using a Luna C₁₈(2) column (75×30 mm, 5 μm particle size) sold by Phenomenex (pn: 00C-4252-U0-AX), and a dual gradient run from 50-99% mobile phase B over 15.0 minutes (mobile phase A=water (5 mM HCl), mobile phase B=acetonitrile, flow rate=50 mL/min, injection volume=950 μL and column temperature=25° C.) giving the intended lactam product, 6-(2,6-dimethylphenyl)-2,2-dioxo-12-(2-tetrahydropyran-4-ylethyl)-11-[2-[1-(trifluoromethyl)cyclopropyl]ethyl]-9-oxa-2λ⁶-thia-3,5,12,19-tetrazatricyclo[12.3.1.14,8]nonadeca-1(18),4(19),5,7,14,16-hexaen-13-one as a white solid (71.7 mg, 23%)¹H NMR (400 MHz, Chloroform-d) δ 8.50 (t, J 1.8 Hz, 1H), 8.12 (d, J 7.8 Hz, 1H), 7.74 (dt, J 7.7, 1.4 Hz, 1H), 7.62 (t, J 7.8 Hz, 1H), 7.21 (t, J 7.6 Hz, 1H), 7.00 (d, J 7.6 Hz, 2H), 6.14 (s, 1H), 5.28 (dd, J 10.7, 3.9 Hz, 1H), 4.08-3.93 (m, 3H), 3.93-3.75 (m, 2H), 3.47-3.35 (m, 2H), 2.98 (d, J 2.9 Hz, 1H), 1.92 (d, J 30.5 Hz, 6H), 1.84-1.74 (m, 1H), 1.69 (dt, J 15.0, 7.5 Hz, 4H), 1.53 (d, J 3.8 Hz, 1H), 1.47-1.34 (m, 3H), 0.99 (d, J 3.3 Hz, 1H), 0.95-0.84 (m, 2H), 0.49-0.39 (m, 2H). ESI-MS m/z calc. 672.25934, found 673.1 (M+1)⁺; Retention time: 1.85 minutes (LC method A). The reverse phase HPLC also provided 6-(2,6-dimethylphenyl)-2,2-dioxo-9-(2-tetrahydropyran-4-ylethyl)-10-[2-[1-(trifluoromethyl)cyclopropyl]ethyl]-12-oxa-2λ⁶-thia-3,5,9,19-tetrazatricyclo[12.3.1.14,8]nonadeca-1(18),4(19),5,7,14,16-hexaen-13-one as a white solid (5.49 mg, 2%) ESI-MS m/z calc. 672.25934, found 673.1 (M+1)⁺; Retention time: 1.79 minutes (LC method A).

### Step 6: 6-(2,6-Dimethylphenyl)-2,2-dioxo-12-(2-tetrahydropyran-4-ylethyl)-11-[2-[1-(trifluoromethyl)cyclopropyl]ethyl]-9-oxa-2λ⁶-thia-3,5,12,19-tetrazatricyclo[12.3.1.14,8]nonadeca-1(18),4(19),5,7,14,16-hexaen-13-one, enantiomer 1 (Compound 72), and 6-(2,6-dimethylphenyl)-2,2-dioxo-12-(2-tetrahydropyran-4-ylethyl)-11-[2-[1-(trifluoromethyl)cyclopropyl]ethyl]-9-oxa-2λ⁶-thia-3,5,12,19-tetrazatricyclo[12.3.1.14,8]nonadeca-1(18),4(19),5,7,14,16-hexaen-13-one, enantiomer 2 (Compound 73)

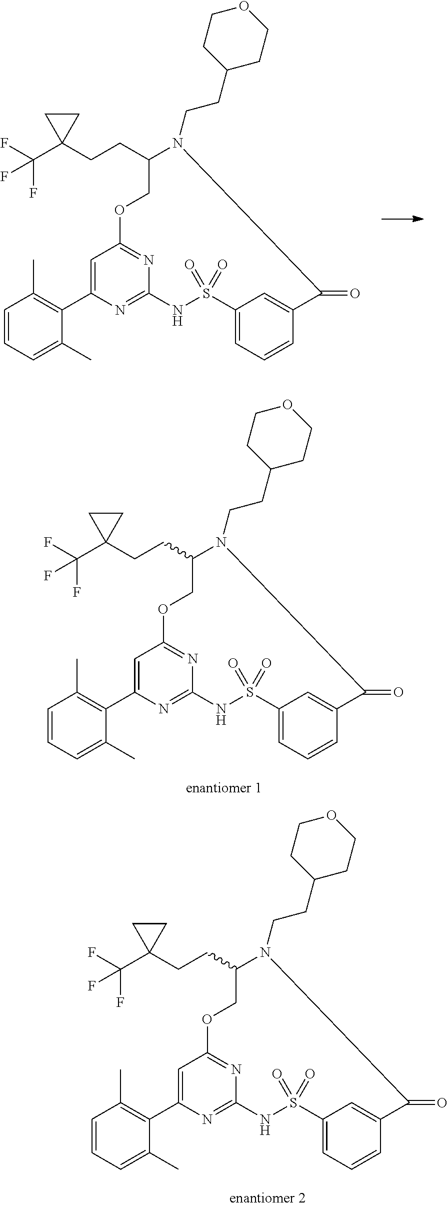 [SMILES: Cc1cccc(C)c1-c1cc2nc(n1)NS(=O)(=O)c1cccc(c1)C(=O)N(CCC1CCOCC1)C(CCC1(C(F)(F)F)CC1)CO2 ; Cc1cccc(C)c1-c1cc2nc(n1)NS(=O)(=O)c1cccc(c1)C(=O)N(CCC1CCOCC1)C(CCC1(C(F)(F)F)CC1)CO2 ; Cc1cccc(C)c1-c1cc2nc(n1)NS(=O)(=O)c1cccc(c1)C(=O)N(CCC1CCOCC1)C(CCC1(C(F)(F)F)CC1)CO2]

6-(2,6-Dimethylphenyl)-12-[2-(oxan-4-yl)ethyl]-11-{2-[1-(trifluoromethyl)cyclopropyl]ethyl}-9-oxa-2λ⁶-thia-3,5,12,19-tetraazatricyclo[12.3.1.14,8]nonadeca-1(17),4,6,8(19),14(18),15-hexaene-2,2,13-trione (64 mg, 0.09513 mmol) was subjected to chiral SFC using.a Phenomenex LUX-1 (250×21.1 mm, 5 μm) column, a mobile phase comprised of 26% MeCN/MeOH (90:10, v:v, no modifier) and 74% C02, with a flow of 70 mL/min, at a concentration of 18 mg/mL in MeCN/MeOH (90:10, v:v, no modifier), with an injection volume of 500 μL, a pressure of 100 bar and a wavelength of 210 nm, to give two enantiomers: Enantiomer 1, Peak 1, 6-(2,6-dimethylphenyl)-2,2-dioxo-12-(2-tetrahydropyran-4-ylethyl)-11-[2-[1-(trifluoromethyl)cyclopropyl]ethyl]-9-oxa-2λ⁶-thia-3,5,12,19-tetrazatricyclo[12.3.1.14,8]nonadeca-1(18),4(19),5,7,14,16-hexaen-13-one (15.5 mg, 48%) isolated as a white solid. ¹H NMR (400 MHz, DMSO-d₆) δ 13.05 (s, 1H), 8.37 (s, 1H), 7.89 (s, 1H), 7.65 (s, 2H), 7.26 (s, 1H), 7.12 (d, J 7.4 Hz, 2H), 6.37 (s, 1H), 5.10 (dd, J 10.6, 4.0 Hz, 1H), 4.30 (s, 1H), 3.92-3.73 (m, 3H), 3.58 (t, J=11.6 Hz, 1H), 3.31 (s, 2H), 3.15 (d, J 11.3 Hz, 1H), 2.06 (d, J 11.5 Hz, 6H), 1.90-1.73 (m, 1H), 1.63 (dd, J=26.8, 11.6 Hz, 6H), 1.45 (d, J=12.0 Hz, 1H), 1.24 (t, J=11.0 Hz, 2H), 1.03-0.91 (m, 1H), 0.74 (dd, J=9.1, 6.0 Hz, 2H), 0.54 (s, 2H). ESI-MS m/z calc. 672.25934, found 673.4 (M+1)⁺; Retention time: 1.86 minutes (LC method A); and enantiomer 2, peak 2, 6-(2,6-dimethylphenyl)-2,2-dioxo-12-(2-tetrahydropyran-4-ylethyl)-11-[2-[1-(trifluoromethyl)cyclopropyl]ethyl]-9-oxa-2λ⁶-thia-3,5,12,19-tetrazatricyclo[12.3.1.14,8]nonadeca-1(18),4(19),5,7,14,16-hexaen-13-one (17.2 mg, 54%) isolated as a white solid, ¹H NMR (400 MHz, DMSO-d₆) δ 8.37 (s, 1H), 7.89 (s, 1H), 7.65 (s, 2H), 7.25 (d, J 8.0 Hz, 1H), 7.13 (d, J 7.6 Hz, 2H), 6.37 (s, 1H), 5.10 (dd, J 10.7, 4.0 Hz, 1H), 4.30 (t, J 10.9 Hz, 1H), 3.93-3.70 (m, 3H), 3.58 (t, J 11.8 Hz, 1H), 3.15 (d, J 11.4 Hz, 1H), 2.06 (d, J 11.1 Hz, 6H), 1.88-1.73 (m, 1H), 1.61 (dt, J 29.0, 11.1 Hz, 6H), 1.44 (t, J=13.2 Hz, 1H), 1.25 (q, J=10.7, 9.9 Hz, 2H), 1.03-0.90 (m, 1H), 0.73 (td, J=9.7, 5.5 Hz, 2H), 0.54 (s, 2H) ESI-MS m/z calc. 672.25934, found 673.5 (M+1)⁺; Retention time: 1.85 minutes (LC method A).

### Example 55: Preparation of Compound 75

### Step 1: 6-[[4-[(2R)-2-Amino-4,4-dimethyl-pentoxy]-6-(2,6-dimethylphenyl)pyrimidin-2-yl]sulfamoyl]pyridine-2-carboxylic acid

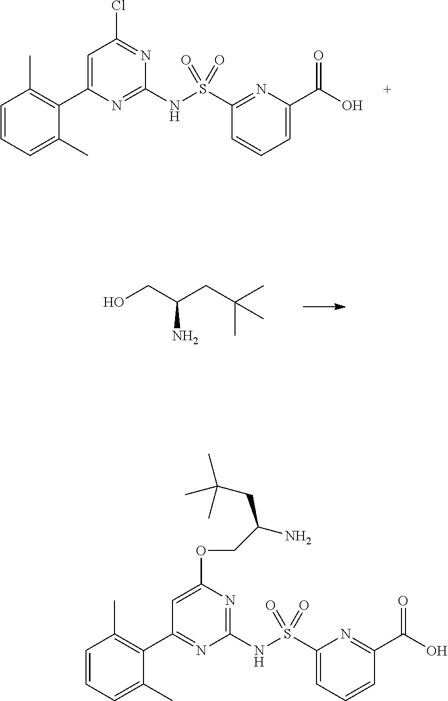

6-[[4-chloro-6-(2,6-dimethylphenyl)pyrimidin-2-yl]sulfamoyl]pyridine-2-carboxylic acid (5.03 g, 12.01 mmol) and (2R)-2-amino-4,4-dimethyl-pentan-1-ol (hydrochloride salt) (2.05 g, 12.23 mmol) were combined in THE (35 mL). To the resulting suspension (hard to stir), sodium tert-butoxide (4.62 g, 48.07 mmol) was added in 3 equal portions resulting in partial dissolution of the solids and a slightly exothermic reaction. The mixture was stirred at room temperature for 5 hours (cloudy suspension). More (2R)-2-amino-4,4-dimethyl-pentan-1-ol (hydrochloride salt) (338 mg, 2.016 mmol) and sodium tert-butoxide (Sodium salt) (610 mg, 6.347 mmol) were added and the mixture was stirred for an additional 1.5 h. The reaction was diluted with ethyl acetate (80 mL), HCl (75 mL of 1 M, 75.00 mmol) and brine (50 mL) and the resulting two phases separated. The aqueous phase was further extracted with EtOAc (3×20 mL). The combined organic extracts were dried over sodium sulfate and concentrated. The residue was triturated in a 1:3 EtOAc: hexanes mixture and stirred in this solvent mixture over the weekend. The solid was filtered and dried to give 6-[[4-[(2R)-2-amino-4,4-dimethyl-pentoxy]-6-(2,6-dimethylphenyl)pyrimidin-2-yl]sulfamoyl]pyridine-2-carboxylic acid (hydrochloride salt) (6.397 g, 97%) as a white solid. ESI-MS m/z calc. 513.2046, found 514.6 (M+1)⁺; Retention time: 1.05 minutes; LC method A. ¹H NMR (400 MHz, DMSO-d₆) δ 13.36 (broad s, 1H), 8.43-7.87 (m, 6H), 7.28 (t, J 7.6 Hz, 1H), 7.14 (d, J 7.6 Hz, 2H), 6.31 (s, 1H), 4.20 (dd, J=12.3, 2.9 Hz, 1H), 4.09-3.91 (m, 1H), 3.61 (s, 1H), 2.03 (s, 6H), 1.57 (dd, J 14.7, 7.3 Hz, 1H), 1.46 (dd, J=14.6, 3.7 Hz, 1H), 0.93 (s, 9H).

### Step 2: 6-[[4-(2,6-Dimethylphenyl)-6-[(2R)-4,4-dimethyl-2-(pyrimidin-2-ylmethylamino)pentoxy]pyrimidin-2-yl]sulfamoyl]pyridine-2-carboxylic acid

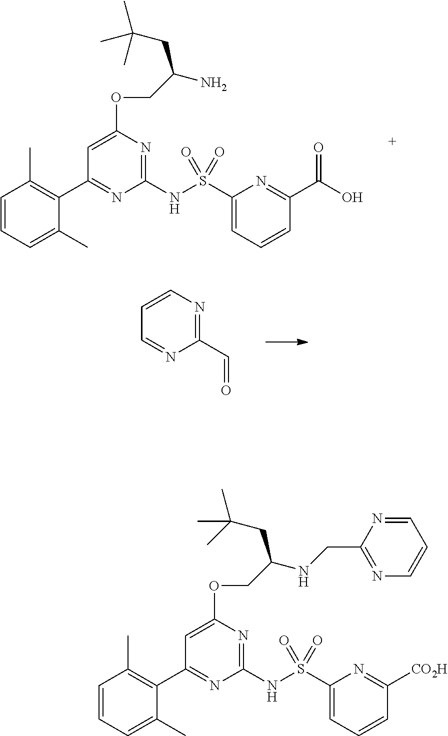 [SMILES: Cc1cccc(C)c1-c1cc(OC[C@H](N)CC(C)(C)C)nc(NS(=O)(=O)c2cccc(C(=O)O)n2)n1 ; O=Cc1ncccn1 ; Cc1cccc(C)c1-c1cc(OC[C@@H](CC(C)(C)C)NCc2ncccn2)nc(NS(=O)(=O)c2cccc(C(=O)O)n2)n1]

6-[[4-[(2R)-2-Amino-4,4-dimethyl-pentoxy]-6-(2,6-dimethylphenyl)pyrimidin-2-yl]sulfamoyl]pyridine-2-carboxylic acid (hydrochloride salt) (approximately 51.94 mg, 0.09442 mmol), pyrimidine-2-carbaldehyde (approximately 12.25 mg, 0.1133 mmol), triethylamine (20 μL, 0.1435 mmol), and acetic acid (10 μL, 0.1758 mmol) were combined in dichloromethane (0.3 mL) and stirred for 5 min. Sodium triacetoxyborohydride (80 mg, 0.3775 mmol) was added and the reaction was stirred an additional 1 h. The reaction mixture was partitioned between ethyl acetate and a 1M HCl solution. The organics were separated, washed with brine, dried over sodium sulfate and evaporated. The crude material was purified by reverse phase HPLC utilizing a gradient of 1-99% acetonitrile in 5 mM aqueous HCl to yield 6-[[4-(2,6-dimethylphenyl)-6-[(2R)-4,4-dimethyl-2-(pyrimidin-2-ylmethylamino)pentoxy]pyrimidin-2-yl]sulfamoyl]pyridine-2-carboxylic acid (hydrochloride salt) (19.6 mg, 34%). ESI-MS m/z calc. 605.24207, found 606.7 (M+1)⁺; Retention time: 0.47 minutes; LC method D.

### Step 3: (11R)-6-(2,6-Dimethylphenyl)-11-(2,2-dimethylpropyl)-12-[(pyrimidin-2-yl)methyl]-9-oxa-2)⁶-thia-3,5,12,18,19-pentaazatricyclo[12.3.1.14,8]nonadeca-1(18),4(19),5,7,14,16-hexaene-2,2,13-trione (Compound 75)

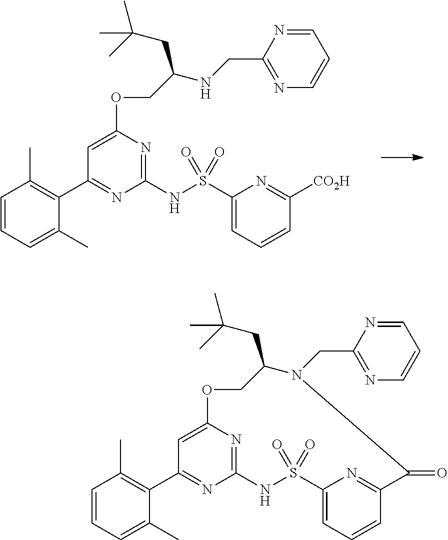 [SMILES: Cc1cccc(C)c1-c1cc(OC[C@@H](CC(C)(C)C)NCc2ncccn2)nc(NS(=O)(=O)c2cccc(C(=O)O)n2)n1 ; Cc1cccc(C)c1-c1cc2nc(n1)NS(=O)(=O)c1cccc(n1)C(=O)N(Cc1ncccn1)[C@H](CC(C)(C)C)CO2]

6-[[4-(2,6-Dimethylphenyl)-6-[(2R)-4,4-dimethyl-2-(pyrimidin-2-ylmethylamino)pentoxy]pyrimidin-2-yl]sulfamoyl]pyridine-2-carboxylic acid (hydrochloride salt) (19.6 mg, 0.03052 mmol), [dimethylamino(triazolo[4,5-b]pyridin-3-yloxy)methylene]-dimethyl-ammonium;hexafluorophosphate (14.5 mg, 0.03813 mmol), and DIEA (27 μL, 0.1550 mmol) were combined in DMF (1 mL) and stirred at room temperature for 30 min. The reaction was filtered and purified by reverse phase HPLC utilizing a gradient of 1-99% acetonitrile in 5 mM aqueous HCl to yield (11R)-6-(2,6-dimethylphenyl)-11-(2,2-dimethylpropyl)-12-[(pyrimidin-2-yl)methyl]-9-oxa-2λ⁶-thia-3,5,12,18,19-pentaazatricyclo[12.3.1.14,8]nonadeca-1(18),4(19),5,7,14,16-hexaene-2,2,13-trione (8.3 mg, 46%) as a white solid. ESI-MS m/z calc. 587.23145, found 589.0 (M+1)⁺; Retention time: 1.52 minutes; LC method A.

### Example 56: Preparation of Compound 76

### Step 1: 6-{[4-(2,6-Dimethylphenyl)-6-{[(2R)-2-[(5-ethoxy-5-oxopentyl)amino]-4,4-dimethylpentyl]oxy}pyrimidin-2-yl]sulfamoyl}pyridine-2-carboxylic acid

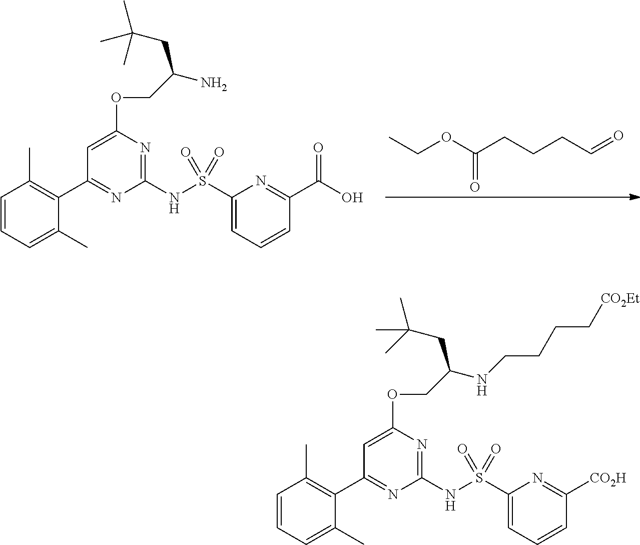

6-[[4-[(2R)-2-amino-4,4-dimethyl-pentoxy]-6-(2,6-dimethylphenyl)pyrimidin-2-yl]sulfamoyl]pyridine-2-carboxylic acid (hydrochloride salt) (100 mg, 0.1818 mmol), ethyl 5-oxopentanoate (36 mg, 0.2497 mmol), and sodium triacetoxyborohydride (156 mg, 0.7361 mmol) were combined in dichloromethane and stirred at room temperature for 2 h. The reaction mixture was partitioned between ethyl acetate and a 1M HCl solution. The organics were separated, washed with brine, dried over sodium sulfate and evaporated. The crude material was purified by reverse phase HPLC utilizing a gradient of 1-99% acetonitrile in 5 mM aqueous HCl to yield 6-{[4-(2,6-dimethylphenyl)-6-{[(2R)-2-[(5-ethoxy-5-oxopentyl)amino]-4,4-dimethylpentyl]oxy}pyrimidin-2-yl]sulfamoyl}pyridine-2-carboxylic acid (hydrochloride salt) (37.3 mg, 32%). ESI-MS m/z calc. 641.2883, found 642.9 (M+1)⁺; Retention time: 0.53 minutes; LC method D.

### Step 2: Ethyl 5-[(11R)-6-(2,6-dimethylphenyl)-11-(2,2-dimethylpropyl)-2,2,13-trioxo-9-oxa-2λ⁶-thia-3,5,12,18,19-pentazatricyclo[12.3.1.14,8]nonadeca-1(18),4,6,8(19),14,16-hexaen-12-yl]pentanoate

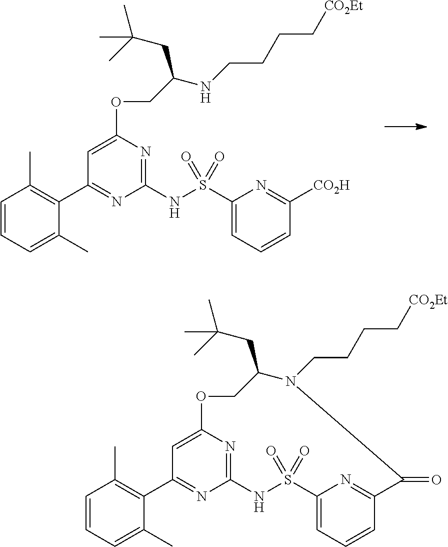 [SMILES: CCOC(=O)CCCCN[C@@H](COc1cc(-c2c(C)cccc2C)nc(NS(=O)(=O)c2cccc(C(=O)O)n2)n1)CC(C)(C)C ; CCOC(=O)CCCCN1C(=O)c2cccc(n2)S(=O)(=O)Nc2nc(cc(-c3c(C)cccc3C)n2)OC[C@H]1CC(C)(C)C]

6-{[4-(2,6-Dimethylphenyl)-6-{[(2R)-2-[(5-ethoxy-5-oxopentyl)amino]-4,4-dimethylpentyl]oxy}pyrimidin-2-yl]sulfamoyl}pyridine-2-carboxylic acid (hydrochloride salt) (17.7 mg, 0.02610 mmol), HATU (13.5 mg, 0.03550 mmol), and DIEA (23 μL, 0.1320 mmol) were combined in DMF (1 mL) and stirred at room temperature for 20 min. The reaction was partitioned between ethyl acetate and a 1M HCl solution. The organics were separated, washed with brine, dried over magnesium sulfate and evaporated to give ethyl 5-[(11R)-6-(2,6-dimethylphenyl)-11-(2,2-dimethylpropyl)-2,2,13-trioxo-9-oxa-2λ⁶-thia-3,5,12,18,19-pentazatricyclo[12.3.1.14,8]nonadeca-1(18),4,6,8(19),14,16-hexaen-12-yl]pentanoate (16.2 mg, 100%) as a clear oil. ESI-MS m/z calc. 623.2778, found 624.8 (M+1)⁺; Retention time: 0.72 minutes; LC method D.

### Step 3: (11R)-6-(2,6-Dimethylphenyl)-11-(2,2-dimethylpropyl)-12-(5-hydroxy-5-methylhexyl)-9-oxa-2λ⁶-thia-3,5,12,18,19-pentaazatricyclo[12.3.1.14,8]nonadeca-1(18),4(19),5,7,14,16-hexaene-2,2,13-trione (Compound 76)

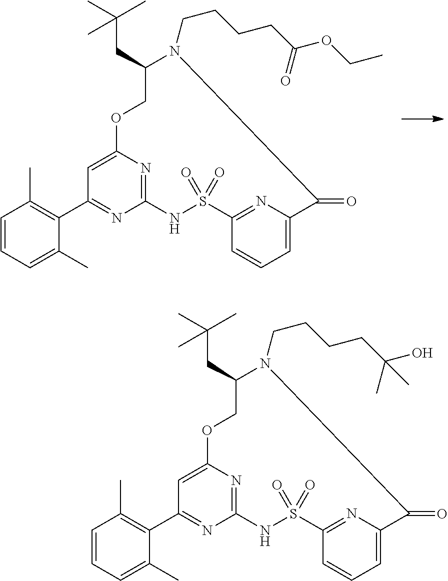 [SMILES: CCOC(=O)CCCCN1C(=O)c2cccc(n2)S(=O)(=O)Nc2nc(cc(-c3c(C)cccc3C)n2)OC[C@H]1CC(C)(C)C ; Cc1cccc(C)c1-c1cc2nc(n1)NS(=O)(=O)c1cccc(n1)C(=O)N(CCCCC(C)(C)O)[C@H](CC(C)(C)C)CO2]

Ethyl 5-[(11R)-6-(2,6-dimethylphenyl)-11-(2,2-dimethylpropyl)-2,2,13-trioxo-9-oxa-2λ⁶-thia-3,5,12,18,19-pentaazatricyclo[12.3.1.14,8]nonadeca-1(18),4(19),5,7,14,16-hexaen-12-yl]pentanoate (25.7 mg, 0.04120 mmol) was dissolved in THF (0.5 mL) and methyl magnesium bromide in diethyl ether (100 μL of 3 M, 0.3000 mmol) was added. The reaction was stirred at room temperature for 1 h. The reaction was quenched with a saturated ammonium chloride solution and extracted with ethyl acetate. The organics were separated, washed with brine, dried over sodium sulfate and evaporated. The crude material was purified by reverse phase HPLC utilizing a gradient of 1-99% acetonitrile in 5 mM aqueous HCl to yield (11R)-6-(2,6-dimethylphenyl)-11-(2,2-dimethylpropyl)-12-(5-hydroxy-5-methylhexyl)-9-oxa-2λ⁶-thia-3,5,12,18,19-pentaazatricyclo[12.3.1.14,8]nonadeca-1(18),4(19),5,7,14,16-hexaene-2,2,13-trione (5.3 mg, 21%). ESI-MS m/z calc. 609.29846, found 610.3 (M+1)⁺; Retention time: 1.71 minutes; LC method A.

### Example 57: Preparation of Compound 77

### Step 1: (11R)-6-(2,6-Dimethylphenyl)-11-isobutyl-2,2-dioxo-12-(pyrimidin-2-ylmethyl)-9-oxa-2λ⁶-thia-3,5,12,19-tetrazatricyclo[12.3.1.14,8]nonadeca-1(18),4(19),5,7,14,16-hexaen-13-one (Compound 77)

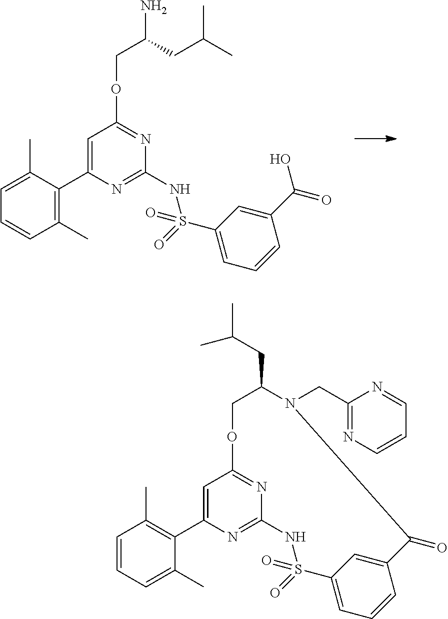

To a solution of 3-[[4-[(2R)-2-amino-4-methyl-pentoxy]-6-(2,6-dimethylphenyl)pyrimidin-2-yl]sulfamoyl]benzoic acid (50 mg, 0.07020 mmol) and pyrimidine-2-carbaldehyde (approximately 8.347 mg, 0.07722 mmol) in THE (280.8 μL) and DCE (280.8 μL) was added sodium triacetoxyborohydride (approximately 74.39 mg, 0.3510 mmol). The reaction was stirred for 2 h before concentrating under a stream of air. The crude residue was dissolved in DMSO and acidified with hydrochloric acid (6 M). The sample was purified by reverse phase HPLC (Phenomenex Luna C₁₈ column (75×30 mm, 5 μm particle size), gradient: 1-99% acetonitrile in water (5 mM HCl) over 15.0 minutes) which afforded the intermediate secondary amine. The intermediate was dissolved in DMF (1.120 mL). HATU (approximately 40.04 mg, 0.1053 mmol) was added and the reaction was stirred for 10 min. triethylamine (approximately 21.31 mg, 29.35 μL, 0.2106 mmol) was added and the reaction was stirred for 30 min. The sample was purified by reverse phase HPLC (Phenomenex Luna C₁₈ column (75×30 mm, 5 μm particle size), gradient: 1-99% acetonitrile in water (5 mM HCl) over 15.0 minutes) which afforded (11R)-6-(2,6-dimethylphenyl)-11-isobutyl-2,2-dioxo-12-(pyrimidin-2-ylmethyl)-9-oxa-2λ⁶-thia-3,5,12,19-tetrazatricyclo[12.3.1.14,8]nonadeca-1(18),4(19),5,7,14,16-hexaen-13-one as a white solid. ESI-MS m/z calc. 572.2206, found 573.57 (M+1)⁺; Retention time: 1.63 minutes; LC method A.

### Example 58: Preparation of Compound 78

### Step 1: 4-[[(11R)-6-(2,6-Dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]benzonitrile (Compound 78)

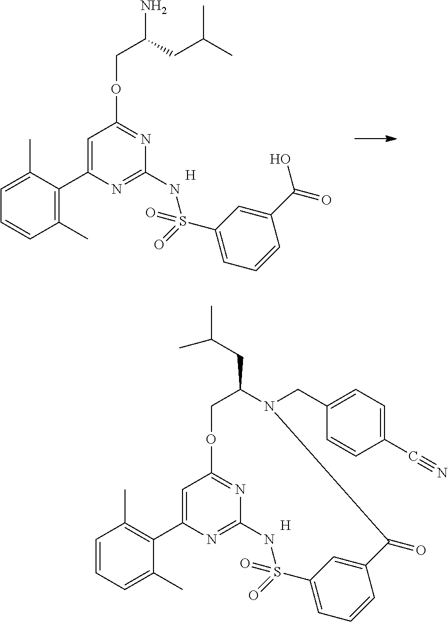

To a solution of 3-[[4-[(2R)-2-amino-4-methyl-pentoxy]-6-(2,6-dimethylphenyl)pyrimidin-2-yl]sulfamoyl]benzoic acid (50 mg, 0.07020 mmol) and 4-formylbenzonitrile (approximately 10.13 mg, 0.07722 mmol) in THE (280.8 μL) and DCE (280.8 μL) was added sodium triacetoxyborohydride (approximately 74.39 mg, 0.3510 mmol). The reaction was stirred for 2 h before concentrating under a steady stream of air. The crude residue was dissolved in DMSO and acidified with 6 N hydrochloric acid. The sample was purified by reverse phase HPLC (Phenomenex Luna C₁₈ column (75×30 mm, 5 μm particle size), gradient: 1-99% acetonitrile in water (5 mM HCl) over 15.0 minutes) which afforded the intermediate secondary amine. The intermediate was dissolved in DMF (1.120 mL). HATU (approximately 40.04 mg, 0.1053 mmol) was added and the reaction was stirred for 10 min. triethylamine (approximately 21.31 mg, 29.35 μL, 0.2106 mmol) was added and the reaction was stirred for 30 min. The sample was purified by reverse phase HPLC (Phenomenex Luna Cis column (75×30 mm, 5 μm particle size), gradient: 1-99% acetonitrile in water (5 mM HCl) over 15.0 minutes) which afforded 4-[[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]benzonitrile as a white solid. ESI-MS m/z calc. 595.22534, found 596.52 (M+1)⁺; Retention time: 1.84 minutes; LC method A.

### Example 59: Preparation of Compound 79

### Step 1: (11R)-6-(2,6-Dimethylphenyl)-11-isobutyl-2,2-dioxo-12-(2-tetrahydropyran-4-ylethyl)-9-oxa-2λ⁶-thia-3,5,12,19-tetrazatricyclo[12.3.1.14,8]nonadeca-1(18),4(19),5,7,14,16-hexaen-13-one (Compound 79)

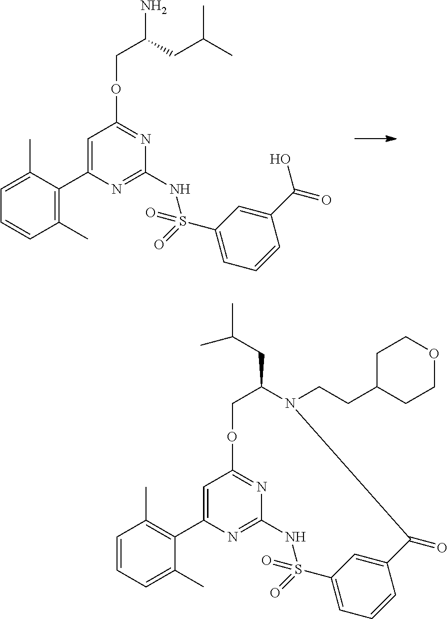

To a solution of 3-[[4-[(2R)-2-amino-4-methyl-pentoxy]-6-(2,6-dimethylphenyl)pyrimidin-2-yl]sulfamoyl]benzoic acid (50 mg, 0.07020 mmol) and 2-tetrahydropyran-4-ylacetaldehyde (approximately 9.897 mg, 0.07722 mmol) in THE (280.8 μL) and DCE (280.8 μL) was added sodium triacetoxyborohydride (approximately 74.39 mg, 0.3510 mmol). The reaction was stirred for 2 h before concentrating under a steady stream of air. The crude residue was dissolved in DMSO and acidified with hydrochloric acid (6 M). The sample was purified by reverse phase HPLC (Phenomenex Luna C₁₈ column (75×30 mm, 5 μm particle size), gradient: 1-99% acetonitrile in water (5 mM HCl) over 15.0 minutes) which afforded the intermediate secondary amine. The intermediate was dissolved in DMF (1.120 mL). HATU (approximately 40.04 mg, 0.1053 mmol) was added and the reaction was stirred for 10 min. triethylamine (approximately 21.31 mg, 29.35 μL, 0.2106 mmol) was added and the reaction was stirred for 30 min. The sample was purified by reverse phase HPLC (Phenomenex Luna Cis column (75×30 mm, 5 μm particle size), gradient: 1-99% acetonitrile in water (5 mM HCl) over 15.0 minutes) which afforded (11R)-6-(2,6-dimethylphenyl)-11-isobutyl-2,2-dioxo-12-(2-tetrahydropyran-4-ylethyl)-9-oxa-2λ⁶-thia-3,5,12,19-tetrazatricyclo[12.3.1.14,8]nonadeca-1(18),4(19),5,7,14,16-hexaen-13-one as a white solid. ESI-MS m/z calc. 592.2719, found 593.59 (M+1)⁺; Retention time: 1.81 minutes; LC method A.

### Example 60: Preparation of Compound 80 and Compound 81

### Step 1: 3-[[4-[(2R)-2-(2-Benzyloxyethylamino)-4-methyl-pentoxy]-6-(2,6-dimethylphenyl)pyrimidin-2-yl]sulfamoyl]benzoic acid

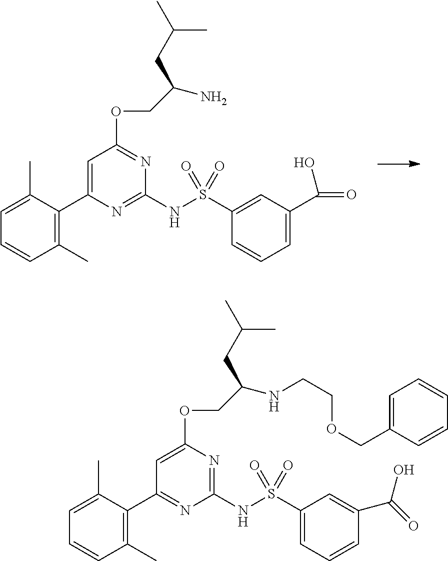 [SMILES: Cc1cccc(C)c1-c1cc(OC[C@H](N)CC(C)C)nc(NS(=O)(=O)c2cccc(C(=O)O)c2)n1 ; Cc1cccc(C)c1-c1cc(OC[C@@H](CC(C)C)NCCOCc2ccccc2)nc(NS(=O)(=O)c2cccc(C(=O)O)c2)n1]

3-[[4-[(2R)-2-Amino-4-methyl-pentoxy]-6-(2,6-dimethylphenyl)pyrimidin-2-yl]sulfamoyl]benzoic acid (hydrochloride salt) (50 mg, 0.09345 mmol) was combined with 2-benzyloxyacetaldehyde (approximately 42.11 mg, 39.39 μL, 0.2804 mmol) in DCE. Acetic acid (approximately 33.67 mg, 31.88 μL, 0.5607 mmol) was added followed by sodium cyanoborohydride (approximately 23.49 mg, 0.3738 mmol) after 10 minutes. The reaction mixture was then stirred for 1-16 hours, partially concentrated then diluted with methanol, filtered and purified by reverse phase HPLC (1-70% ACN, HCl modifier, 15 min run) to give the 3-[[4-[(2R)-2-(2-benzyloxyethylamino)-4-methyl-pentoxy]-6-(2,6-dimethylphenyl)pyrimidin-2-yl]sulfamoyl]benzoic acid (20.4 mg, 35%) after drying. ESI-MS m/z calc. 632.26685, found 633.5 (M+1)⁺; Retention time: 0.52 minutes; LC method D.

### Step 2: (11R)-12-[2-(Benzyloxy)ethyl]-6-(2,6-dimethylphenyl)-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 81)

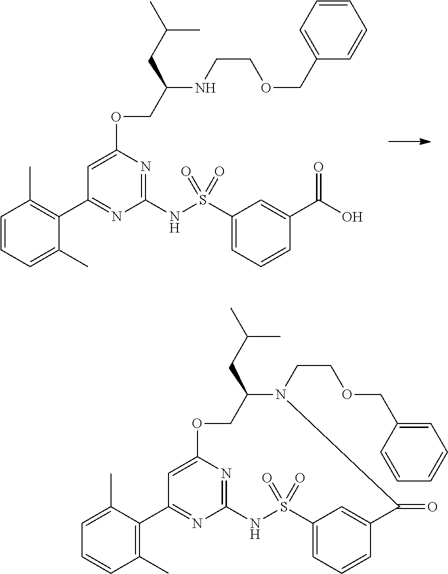 [SMILES: Cc1cccc(C)c1-c1cc(OC[C@@H](CC(C)C)NCCOCc2ccccc2)nc(NS(=O)(=O)c2cccc(C(=O)O)c2)n1 ; Cc1cccc(C)c1-c1cc2nc(n1)NS(=O)(=O)c1cccc(c1)C(=O)N(CCOCc1ccccc1)[C@H](CC(C)C)CO2]

3-[[4-[(2R)-2-(2-Benzyloxyethylamino)-4-methyl-pentoxy]-6-(2,6-dimethylphenyl)pyrimidin-2-yl]sulfamoyl]benzoic acid was dissolved in DMF. HATU was added. After stirring at room temperature for 5 minutes, triethylamine was added. After 5 minutes of stirring, the product was isolated by reverse phase HPLC using a Luna C₁₈ (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-12-[2-(benzyloxy)ethyl]-6-(2,6-dimethylphenyl)-11-(2-methylpropyl)-9-oxa-2λ⁶-thia-3,5,12,19-tetraazatricyclo[12.3.1.14,8]nonadeca-1(17),4,6,8(19),14(18),15-hexaene-2,2,13-trione (6.2 mg) was obtained. ESI-MS m/z calc. 614.2563, found 615.3 (M+1)⁺; Retention time: 1.99 minutes; LC method A.

### Step 3: (11R)-6-(2,6-Dimethylphenyl)-12-(2-hydroxyethyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 80)

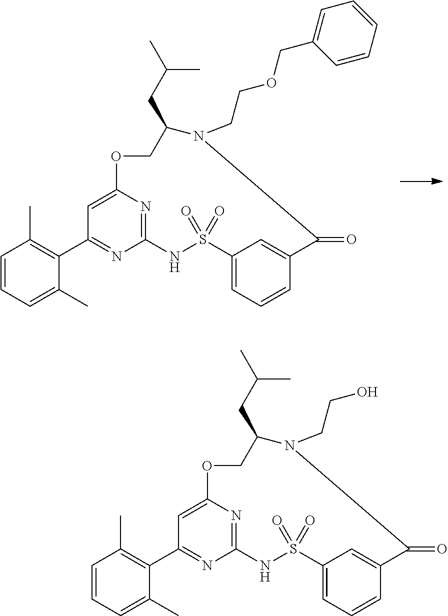 [SMILES: Cc1cccc(C)c1-c1cc2nc(n1)NS(=O)(=O)c1cccc(c1)C(=O)N(CCOCc1ccccc1)[C@H](CC(C)C)CO2 ; Cc1cccc(C)c1-c1cc2nc(n1)NS(=O)(=O)c1cccc(c1)C(=O)N(CCO)[C@H](CC(C)C)CO2]

(11R)-12-(2-Benzyloxyethyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (10.6 mg, 0.01724 mmol) and dihydroxypalladium (3 mg, 0.004273 mmol) in a nitrogen purged vial were combined in methanol (1 mL) and hydrogen from a balloon was bubbled through the reaction mixture for 30 minutes, after which the reaction mixture was allowed to stir at room temperature for an additional 30 minutes with the balloon in place. After this time, the reaction vessel was purged with nitrogen, and the contents were diluted with methanol, filtered through Celite and concentrated. The resulting residue was dissolved in 1:1 DMSO/methanol, filtered a second time, and purified by reverse phase HPLC (1-99% ACN in water, HCl modifier, 15 min run) and dried to give (11R)-6-(2,6-dimethylphenyl)-12-(2-hydroxyethyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (7.2 mg, 80%). ESI-MS m/z calc. 524.20935, found 525.5 (M+1)⁺; Retention time: 1.45 minutes; LC method A.

### Example 61: Preparation of Compound 82

### Step 1: 3-[[4-(2,6-Dimethylphenyl)-6-[(2R)-2-[(4-methoxy-2-pyridyl)methylamino]-4-methyl-pentoxy]pyrimidin-2-yl]sulfamoyl]benzoic acid

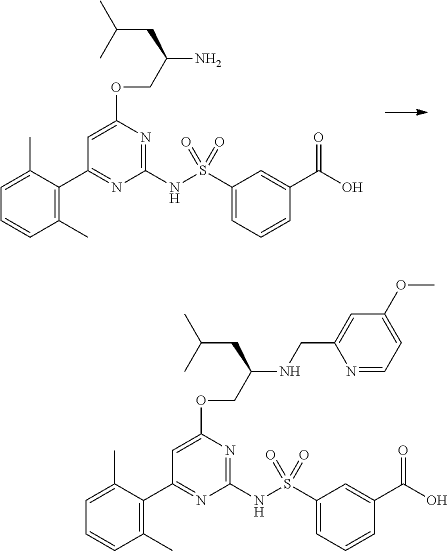 [SMILES: Cc1cccc(C)c1-c1cc(OC[C@H](N)CC(C)C)nc(NS(=O)(=O)c2cccc(C(=O)O)c2)n1 ; COc1ccnc(CN[C@@H](COc2cc(-c3c(C)cccc3C)nc(NS(=O)(=O)c3cccc(C(=O)O)c3)n2)CC(C)C)c1]

3-[[4-[(2R)-2-Amino-4-methyl-pentoxy]-6-(2,6-dimethylphenyl)pyrimidin-2-yl]sulfamoyl]benzoic acid (hydrochloride salt) (50 mg, 0.09345 mmol) and 4-methoxypyridine-2-carbaldehyde (20 mg, 0.1458 mmol) were combined and suspended in dichloromethane (0.5 mL). Sodium triacetoxyborohydride (60 mg, 0.2831 mmol) was added. The reaction mixture was allowed to stir at room temperature overnight. The reaction mixture was purified by reverse phase HPLC using a Luna C₁₈(2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. 3-[[4-(2,6-dimethylphenyl)-6-[(2R)-2-[(4-methoxy-2-pyridyl)methylamino]-4-methyl-pentoxy]pyrimidin-2-yl]sulfamoyl]benzoic acid (50 mg, 86%) was obtained. ESI-MS m/z calc. 619.24646, found 620.3 (M+1)⁺; Retention time: 1.08 minutes; LC method A.

### Step 2: (11R)-6-(2,6-Dimethylphenyl)-11-isobutyl-12-[(4-methoxy-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 82)

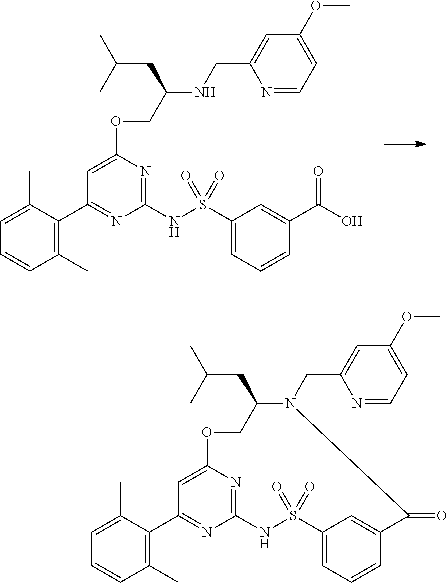 [SMILES: COc1ccnc(CN[C@@H](COc2cc(-c3c(C)cccc3C)nc(NS(=O)(=O)c3cccc(C(=O)O)c3)n2)CC(C)C)c1 ; COc1ccnc(CN2C(=O)c3cccc(c3)S(=O)(=O)Nc3nc(cc(-c4c(C)cccc4C)n3)OC[C@H]2CC(C)C)c1]

3-[[4-(2,6-dimethylphenyl)-6-[(2R)-2-[(4-methoxy-2-pyridyl)methylamino]-4-methyl-pentoxy]pyrimidin-2-yl]sulfamoyl]benzoic acid (50 mg, 0.08068 mmol) was dissolved in DMF (1 mL). COMU (42 mg, 0.09807 mmol) was added. After 5 minutes, triethylamine (35 μL, 0.2511 mmol) was added. The reaction mixture was allowed to stir at room temperature for 30 minutes. The product was purified by reverse phase HPLC using a Luna C₁₈ (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (11R)-6-(2,6-dimethylphenyl)-11-isobutyl-12-[(4-methoxy-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (6.3 mg, 12%) was obtained as a yellowish white solid. ESI-MS m/z calc. 601.2359, found 602.3 (M+1)⁺; Retention time: 1.22 minutes; LC method A.

### Example 62: Preparation of Compound 83

### Step 1: 3-[[4-[(2R)-2-[(3-tert-Butyl-1-methyl-pyrazol-4-yl)methylamino]-4-methyl-pentoxy]-6-(2,6-dimethylphenyl)pyrimidin-2-yl]sulfamoyl]benzoic acid

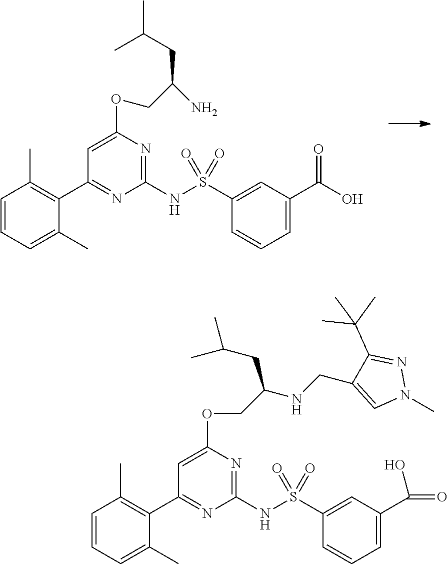 [SMILES: Cc1cccc(C)c1-c1cc(OC[C@H](N)CC(C)C)nc(NS(=O)(=O)c2cccc(C(=O)O)c2)n1 ; Cc1cccc(C)c1-c1cc(OC[C@@H](CC(C)C)NCc2cn(C)nc2C(C)(C)C)nc(NS(=O)(=O)c2cccc(C(=O)O)c2)n1]

3-[[4-[(2R)-2-Amino-4-methyl-pentoxy]-6-(2,6-dimethylphenyl)pyrimidin-2-yl]sulfamoyl]benzoic acid (40 mg, 0.08023 mmol) and 3-tert-butyl-1-methyl-pyrazole-4-carbaldehyde (approximately 40.01 mg, 0.2407 mmol) were combined in DCE with acetic acid (approximately 38.54 mg, 36.50 μL, 0.6418 mmol) and stirred at room temperature for 20 minutes. Sodium cyanoborohydride (approximately 20.17 mg, 0.3209 mmol) was continued at room temperature for 1 hour. At this point, the reaction mixture was quenched with several drops of water, and partially concentrated. The reaction mixture was then dissolved in 1 mL 1:1 DMSO/methanol, then filtered and purified by reverse phase HPLC (1-70% ACN in water, HCl modifier) on a 15 min run. The fractions containing product were concentrated to give as a white solid, 3-[[4-[(2R)-2-[(3-tert-butyl-1-methyl-pyrazol-4-yl)methylamino]-4-methyl-pentoxy]-6-(2,6-dimethylphenyl)pyrimidin-2-yl]sulfamoyl]benzoic acid (hydrochloride salt) (16.2 mg, 29%). ESI-MS m/z calc. 648.3094, found 649.5 (M+1)⁺; Retention time: 0.5 minutes; LC method D.

### Step 2: (11R)-12-[(3-tert-Butyl-1-methyl-pyrazol-4-yl)methyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 83)

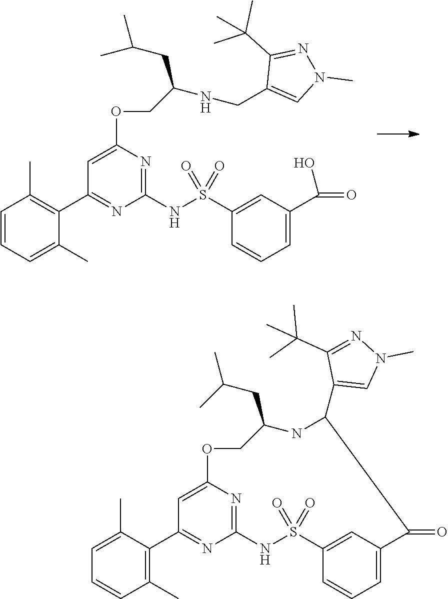 [SMILES: Cc1cccc(C)c1-c1cc(OC[C@@H](CC(C)C)NCc2cn(C)nc2C(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)C(c1cn(C)nc1C(C)(C)C)N[C@H](CC(C)C)CO2]

3-[[4-[(2R)-2-[(3-tert-Butyl-1-methyl-pyrazol-4-yl)methylamino]-4-methyl-pentoxy]-6-(2,6-dimethylphenyl)pyrimidin-2-yl]sulfamoyl]benzoic acid (hydrochloride salt) (14 mg, 0.02043 mmol) was combined with HATU (approximately 10.10 mg, 0.02656 mmol) in DMF (1 mL) and DIPEA (approximately 13.20 mg, 17.79 μL, 0.1021 mmol) was added. The reaction mixture was then stirred at room temperature for 18 h. The reaction was filtered and purified by reverse phase HPLC (1-99% ACN in water, HCl modifier, 15 min run) to give (11R)-12-[(3-tert-butyl-1-methyl-pyrazol-4-yl)methyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (4.3 mg, 32%). ESI-MS m/z calc. 630.2988, found 631.6 (M+1)⁺; Retention time: 1.86 minutes; LC method A.

### Example 63: Preparation of Compound 84

### Step 1: (2R)-2-(4-Methoxybutylamino)-4-methyl-pentan-1-ol

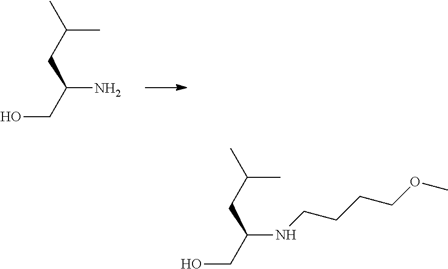

(2R)-2-Amino-4-methyl-pentan-1-ol (39.6 mg, 0.3379 mmol), 4-methoxybutanal (30.9 mg, 0.3026 mmol), and sodium triacetoxyborohydride (132 mg, 0.6228 mmol) were combined in DCM (1 mL) and stirred at room temperature for 3 h. The reaction was evaporated and then partitioned between ethyl acetate and a saturated sodium bicarbonate solution. The organics were separated, washed with brine, dried over sodium sulfate and evaporated. The crude was used directly in the next step. (2R)-2-(4-methoxybutylamino)-4-methyl-pentan-1-ol (20 mg, 29%) ESI-MS m/z calc. 203.18852, found 204.3 (M+1)⁺; Retention time: 0.29 minutes (LC method D).

### Step 2: (11R)-6-(2,6-Dimethylphenyl)-11-isobutyl-12-(4-methoxybutyl)-2,2-dioxo-9-oxa-2λ⁶-thia-3,5,12,19-tetrazatricyclo[12.3.1.14,8]nonadeca-1(18),4 19) 7 14,16-hexaen-13-one (Compound 84)

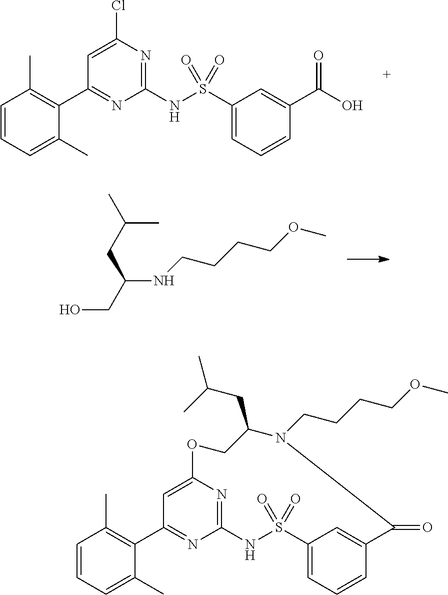

(2R)-2-(4-Methoxybutylamino)-4-methyl-pentan-1-ol (20 mg, 0.09837 mmol) and 3-[[4-chloro-6-(2,6-dimethylphenyl)pyrimidin-2-yl]sulfamoyl]benzoic acid (43 mg, 0.1029 mmol) were combined in THE (1 mL). Sodium tert-butoxide (39 mg, 0.4058 mmol) was added and the reaction mixture was heated at 60° C. for 3 h. The reaction was cooled to room temperature and HATU (79 mg, 0.2078 mmol) was added. The mixture was stirred an additional 1 h and evaporated. The crude material was purified by reverse phase HPLC utilizing a gradient of 1-99% acetonitrile in 5 mM aqueous HCl to yield (11R)-6-(2,6-dimethylphenyl)-11-isobutyl-12-(4-methoxybutyl)-2,2-dioxo-9-oxa-2λ⁶-thia-3,5,12,19-tetrazatricyclo[12.3.1.14,8]nonadeca-1(18),4(19),5,7,14,16-hexaen-13-one (5.7 mg, 9%). ESI-MS m/z calc. 566.2563, found 567.5 (M+1)⁺; Retention time: 1.76 minutes; LC method A.

### Example 64: Preparation of Compound 85

### Step 1: tert-Butyl 4-[2-[(11R)-6-(2,6-dimethylphenyl)-11-isobutyl-2,2,13-trioxo-9-oxa-2)⁶-thia-3,5,12,19-tetrazatricyclo[12.3.1.14,8]nonadeca-1(18),4(19),5,7,14,16-hexaen-12-yl]ethyl]piperidine-1-carboxylate

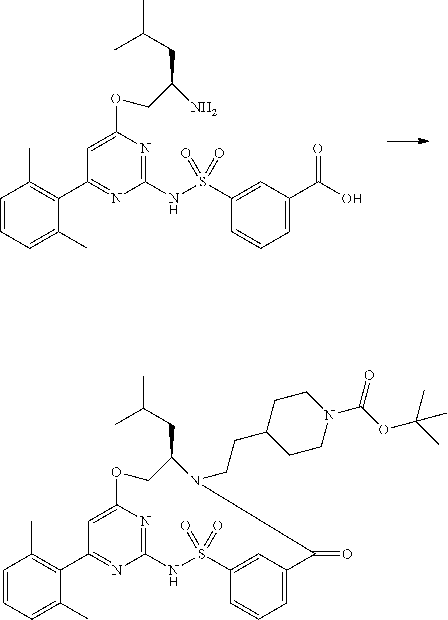 [SMILES: Cc1cccc(C)c1-c1cc(OC[C@H](N)CC(C)C)nc(NS(=O)(=O)c2cccc(C(=O)O)c2)n1 ; Cc1cccc(C)c1-c1cc2nc(n1)NS(=O)(=O)c1cccc(c1)C(=O)N(CCC1CCN(C(=O)OC(C)(C)C)CC1)[C@H](CC(C)C)CO2]

3-[[4-[(2R)-2-Amino-4-methyl-pentoxy]-6-(2,6-dimethylphenyl)pyrimidin-2-yl]sulfamoyl]benzoic acid (hydrochloride salt) (670 mg, 1.252 mmol), tert-butyl 4-(2-oxoethyl)piperidine-1-carboxylate (862 mg, 3.792 mmol), and sodium cyanoborohydride (311 mg, 4.949 mmol) were combined in DCE (5 mL) and stirred at room temperature for 16 h. The reaction was partitioned between ethyl acetate and a 1M HCl solution. The organics were separated, washed with brine, dried over sodium sulfate and evaporated. The crude product was used directly in next step. Crude product was dissolved in DMF (10 mL) and HATU (481 mg, 1.265 mmol) and triethylamine (550 μL, 3.946 mmol) were added. The reaction was stirred at room temperature for 1 h. The reaction was partitioned between ethyl acetate and a 1M HCl solution. The organics were separated, washed with brine, dried over sodium sulfate and evaporated. The crude material was purified by silica gel chromatography eluting with 0-10% dichloromethane in methanol to give tert-butyl 4-[2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]ethyl]piperidine-1-carboxylate (814 mg, 94%) ESI-MS m z calc. 691.34033, found 692.7 (M+1)⁺; Retention time: 0.77 minutes. (LC method D).

### Step 2: (11R)-6-(2,6-Dimethylphenyl)-11-isobutyl-2,2-dioxo-12-[2-(4-piperidyl)ethyl]-9-oxa-2λ⁶-thia-3,5,12,19-tetrazatricyclo[12.3.1.14,8]nonadeca-1(18),4(19),5,7,14,16-hexaen-13-one

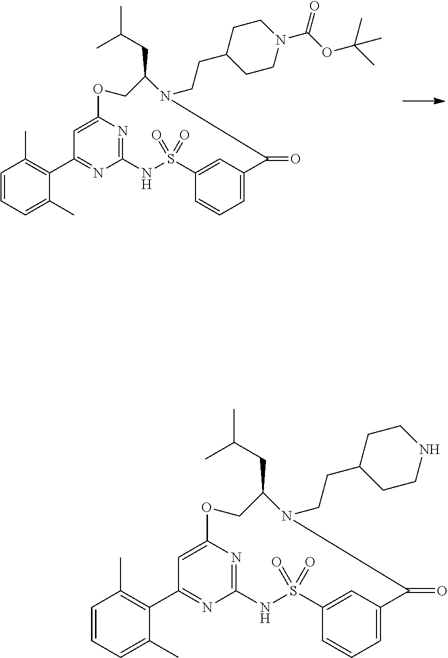 [SMILES: Cc1cccc(C)c1-c1cc2nc(n1)NS(=O)(=O)c1cccc(c1)C(=O)N(CCC1CCN(C(=O)OC(C)(C)C)CC1)[C@H](CC(C)C)CO2 ; Cc1cccc(C)c1-c1cc2nc(n1)NS(=O)(=O)c1cccc(c1)C(=O)N(CCC1CCNCC1)[C@H](CC(C)C)CO2]

tert-Butyl 4-[2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]ethyl]piperidine-1-carboxylate (814 mg, 1.177 mmol) was dissolved in 4M HCl in dioxane (2 mL of 4 M, 8.000 mmol) and stirred at room temperature for 30 min. The reaction was evaporated to dryness. The resulting solid was triturated with ether and the solid was collected. The solid was further dried to give (11R)-6-(2,6-dimethylphenyl)-11-isobutyl-2,2-dioxo-12-[2-(4-piperidyl)ethyl]-9-oxa-2λ⁶-thia-3,5,12,19-tetrazatricyclo[12.3.1.14,8]nonadeca-1(18),4(19),5,7,14,16-hexaen-13-one (hydrochloride salt) (716 mg, 97%). ESI-MS m/z calc. 591.2879, found 592.6 (M+1)⁺; Retention time: 0.47 minutes (LC method D).

### Step 3: (11R)-6-(2,6-Dimethylphenyl)-11-isobutyl-12-[2-(1-isopropyl-4-piperidyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 85)

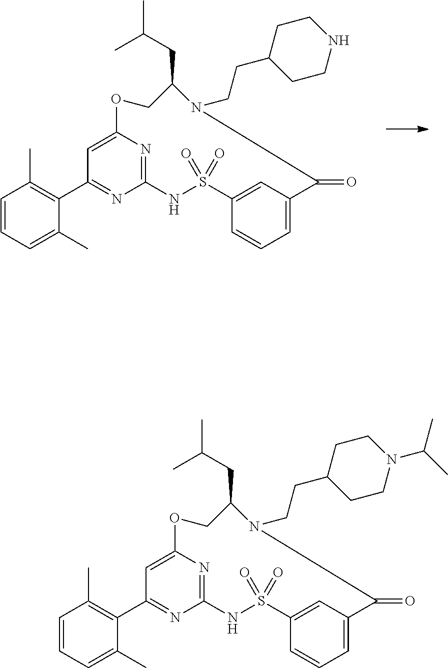 [SMILES: Cc1cccc(C)c1-c1cc2nc(n1)NS(=O)(=O)c1cccc(c1)C(=O)N(CCC1CCNCC1)[C@H](CC(C)C)CO2 ; Cc1cccc(C)c1-c1cc2nc(n1)NS(=O)(=O)c1cccc(c1)C(=O)N(CCC1CCN(C(C)C)CC1)[C@H](CC(C)C)CO2]

(11R)-6-(2,6-Dimethylphenyl)-11-isobutyl-2,2-dioxo-12-[2-(4-piperidyl)ethyl]-9-oxa-2λ⁶-thia-3,5,12,19-tetrazatricyclo[12.3.1.14,8]nonadeca-1(18),4(19),5,7,14,16-hexaen-13-one (hydrochloride salt) (56 mg, 0.08914 mmol), sodium cyanoborohydride (16.4 mg, 0.2610 mmol), and acetone (35 μL, 0.4767 mmol) were combined in methanol (0.5 mL). acetic acid (50 μL, 0.8792 mmol) was added and the reaction mixture was heated at 60° C. for 16 h. The reaction was quenched with 1M HCl (0.1 mL). The diluted with DMSO (0.4 mL), filtered, and purified by reverse phase HPLC utilizing a gradient of 1-99% acetonitrile in 5 mM aqueous HCl to yield (11R)-6-(2,6-dimethylphenyl)-11-isobutyl-12-[2-(1-isopropyl-4-piperidyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (10.4 mg, 17%). ESI-MS m/z calc. 633.3349, found 634.6 (M+1)⁺; Retention time: 1.3 minutes; LC method A.

### Example 65: Preparation of Compound 86

### Step 1: 3-[[4-[(2R)-2-[(4-Bromophenyl)methylamino]-4-methyl-pentoxy]-6-(2,6-dimethylphenyl)pyrimidin-2-yl]sulfamoyl]benzoic acid

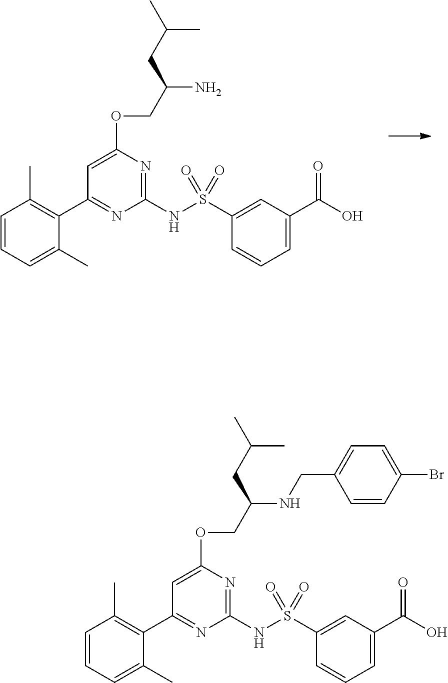 [SMILES: Cc1cccc(C)c1-c1cc(OC[C@H](N)CC(C)C)nc(NS(=O)(=O)c2cccc(C(=O)O)c2)n1 ; Cc1cccc(C)c1-c1cc(OC[C@@H](CC(C)C)NCc2ccc(Br)cc2)nc(NS(=O)(=O)c2cccc(C(=O)O)c2)n1]

3-[[4-[(2R)-2-Amino-4-methyl-pentoxy]-6-(2,6-dimethylphenyl)pyrimidin-2-yl]sulfamoyl]benzoic acid (hydrochloride salt) (340 mg, 0.5973 mmol) and 4-bromobenzaldehyde (166 mg, 0.8972 mmol) were combined and dissolved in dichloromethane (3.5 mL). Sodium triacetoxyborohydride (380 mg, 1.793 mmol) was added. The reaction mixture was allowed to stir overnight at room temperature. It was then diluted with EtOAc (50 mL) and washed with aqueous HCl (1×50 mL) and brine (1×50 mL). The organic layer was dried over sodium sulfate, filtered and concentrated under reduced pressure. The crude product was purified by flash chromatography on a 24 gram silica gel column eluting with a 0-100% EtOAc/hexane gradient; product eluted at 100% EtOAc/hexane with 3-[[4-[(4-bromophenyl)methyl-[(1R)-1-(hydroxymethyl)-3-methyl-butyl]amino]-6-(2,6-dimethylphenyl)pyrimidin-2-yl]sulfamoyl]benzoic acid was the major impurity. 3-[[4-[(2R)-2-[(4-bromophenyl)methylamino]-4-methyl-pentoxy]-6-(2,6-dimethylphenyl)pyrimidin-2-yl]sulfamoyl]benzoic acid (303 mg, 76%) was obtained as a sticky foam. ESI-MS m/z calc. 666.1512, found 667.3 (M+1)⁺; Retention time: 1.31 minutes; LC method A.

### Step 2: (11R)-12-[(4-Bromophenyl)methyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one

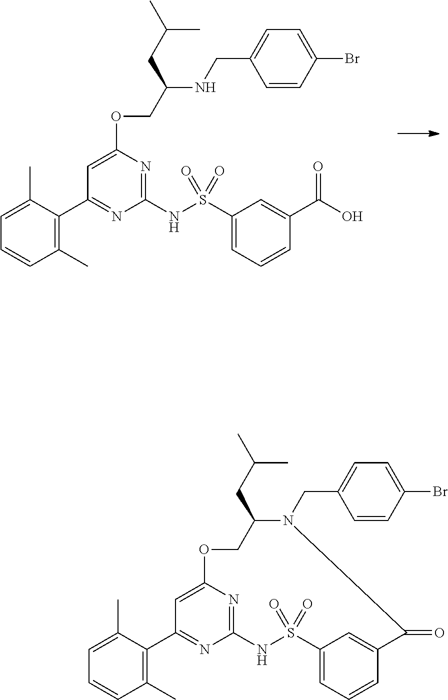 [SMILES: Cc1cccc(C)c1-c1cc(OC[C@@H](CC(C)C)NCc2ccc(Br)cc2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccc(Br)cc1)[C@H](CC(C)C)CO2]

3-[[4-[(2R)-2-[(4-Bromophenyl)methylamino]-4-methyl-pentoxy]-6-(2,6-dimethylphenyl)pyrimidin-2-yl]sulfamoyl]benzoic acid (40 mg, 0.05991 mmol) was dissolved in DMF (1 mL). COMU (31 mg, 0.07238 mmol) was added followed by triethylamine (25 μL, 0.1794 mmol). After 10 minutes of stirring at room temperature, the reaction mixture was diluted with EtOAc (75 mL) and washed with aqueous HCl (1×75 mL) and brine (1×75 mL). The organic layer was dried over sodium sulfate, filtered and concentrated under reduced pressure. The crude product was purified by flash chromatography on a 12 gram silica gel column eluting with a 0-50% EtOAc/hexane gradient over 30 minutes; product eluted at 40%. (11R)-12-[(4-Bromophenyl)methyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (19 mg, 49%) was obtained as a clear colorless residue. ESI-MS m/z calc. 648.14056, found 651.1 (M+3)⁺; Retention time: 2.06 minutes; LC method A.

### Step 3: (11R)-6-(2,6-Dimethylphenyl)-11-isobutyl-12-[[4-(1-methyl-3,6-dihydro-2H-pyridin-4-yl)phenyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 86)

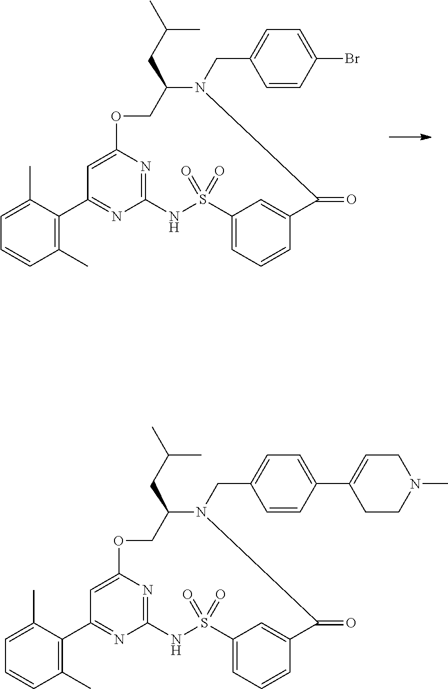 [SMILES: Cc1cccc(C)c1-c1cc2nc(n1)NS(=O)(=O)c1cccc(c1)C(=O)N(Cc1ccc(Br)cc1)[C@H](CC(C)C)CO2 ; Cc1cccc(C)c1-c1cc2nc(n1)NS(=O)(=O)c1cccc(c1)C(=O)N(Cc1ccc(C3=CCN(C)CC3)cc1)[C@H](CC(C)C)CO2]

(11R)-12-[(4-Bromophenyl)methyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (19 mg, 0.02925 mmol) and 1-methyl-4-(4,4,5,5-tetramethyl-1,3,2-dioxaborolan-2-yl)-3,6-dihydro-2H-pyridine (10 mg, 0.04482 mmol) were combined and dissolved in DMF (1 mL). Aqueous potassium carbonate (75 μL of 2 M, 0.1500 mmol) was added followed by Pd(dppf)Cl₂ (2 mg, 0.002449 mmol). The reaction mixture was heated under microwave irradiation at 120° C. for 30 minutes. The product was purified by reverse phase HPLC using a Luna C₁₈ (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 L, and column temperature=25° C. The UV trace at 254 nm was used to collect fractions. (11R)-6-(2,6-dimethylphenyl)-11-isobutyl-12-[[4-(1-methyl-3,6-dihydro-2H-pyridin-4-yl)phenyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (6.55 mg, 34%) was obtained as a brown solid. ESI-MS m/z calc. 665.3036, found 662.5 (M+1)⁺; Retention time: 1.36 minutes; (LC method A).

### Example 66: Preparation of Compound 87

### Step 1: 3-[[4-(2,6-Dimethylphenyl)-6-[(2R)-4-methyl-2-[[4-(1-methyl-4-piperidyl)phenyl]methylamino]pentoxy]pyrimidin-2-yl]sulfamoyl]benzoic acid

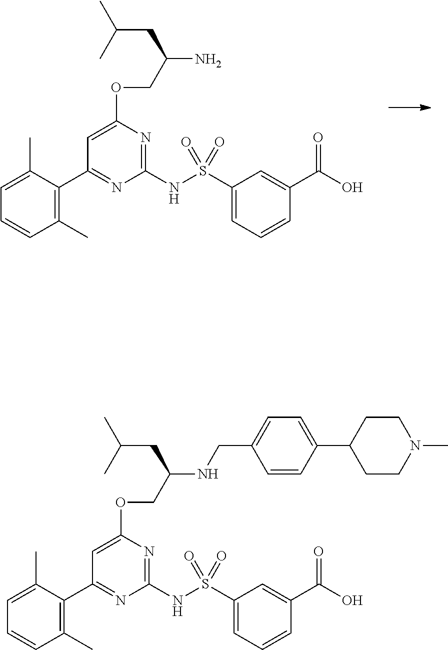 [SMILES: Cc1cccc(C)c1-c1cc(OC[C@H](N)CC(C)C)nc(NS(=O)(=O)c2cccc(C(=O)O)c2)n1 ; Cc1cccc(C)c1-c1cc(OC[C@@H](CC(C)C)NCc2ccc(C3CCN(C)CC3)cc2)nc(NS(=O)(=O)c2cccc(C(=O)O)c2)n1]

3-[[4-[(2R)-2-Amino-4-methyl-pentoxy]-6-(2,6-dimethylphenyl)pyrimidin-2-yl]sulfamoyl]benzoic acid (hydrochloride salt) (50 mg, 0.09345 mmol) was combined with 4-(1-methyl-4-piperidyl)benzaldehyde (29 mg, 0.1427 mmol). The neat mixture was heated briefly with a heat gun until both solids melted together. Dichloromethane (0.5 mL) was added followed by sodium triacetoxyborohydride (60 mg, 0.2831 mmol). The reaction mixture was purified by reverse-phase HPLC using a Luna C₁₈ (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. 3-[[4-(2,6-Dimethylphenyl)-6-[(2R)-4-methyl-2-[[4-(1-methyl-4-piperidyl)phenyl]methylamino]pentoxy]pyrimidin-2-yl]sulfamoyl]benzoic acid (53 mg, 83%) was obtained. ESI-MS m/z calc. 685.3298, found 686.6 (M+1)⁺; Retention time: 0.95 minutes; LC method A.

### Step 2: (11R)-6-(2,6-Dimethylphenyl)-11-isobutyl-12-[[4-(1-methyl-4-piperidyl)phenyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 87)

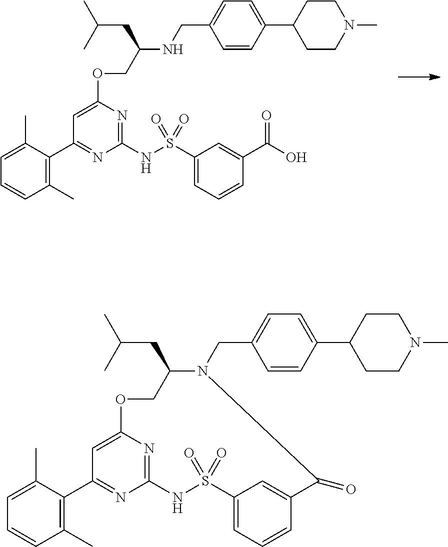 [SMILES: Cc1cccc(C)c1-c1cc(OC[C@@H](CC(C)C)NCc2ccc(C3CCN(C)CC3)cc2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccc(C3CCN(C)CC3)cc1)[C@H](CC(C)C)CO2]

3-[[4-(2,6-Dimethylphenyl)-6-[(2R)-4-methyl-2-[[4-(1-methyl-4-piperidyl)phenyl]methylamino]pentoxy]pyrimidin-2-yl]sulfamoyl]benzoic acid (43 mg, 0.06269 mmol) was dissolved in DMF (2 mL). COMU (32 mg, 0.07472 mmol) was added. After briefly stirring for 5 minutes, triethylamine (25 μL, 0.1794 mmol) was added. After stirring at room temperature for 30 minutes, the reaction mixture was diluted with EtOAc (50 mL) and washed with brine (1×50 mL). The organic layer was dried over sodium sulfate, filtered and concentrated under reduced pressure. The crude product was purified by flash chromatography on 24 gram silica gel column eluting with a 0-20% MeOH/DCM gradient over 30 minutes. (11R)-6-(2,6-Dimethylphenyl)-11-isobutyl-12-[[4-(1-methyl-4-piperidyl)phenyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (28.6 mg, 67%) was obtained as a white solid. ESI-MS m/z calc. 667.3192, found 668.7 (M+1)⁺; Retention time: 1.48 minutes; LC method A. ¹H NMR (400 MHz, DMSO-d₆) δ 8.49 (s, 1H), 7.87 (dt, J 6.4, 2.0 Hz, 1H), 7.60 (d, J 6.6 Hz, 2H), 7.36 (d, J 8.1 Hz, 2H), 7.23 (d, J 7.9 Hz, 2H), 7.18 (t, J 7.6 Hz, 1H), 7.06 (d, J 7.6 Hz, 2H), 6.05 (s, 1H), 5.06 (d, J 5.9 Hz, 1H), 4.80 (d, J 15.6 Hz, 1H), 4.47 (d, J=15.6 Hz, 1H), 4.15-4.02 (m, 3H), 3.14 (s, 1H), 2.46 (s, 4H), 1.98 (d, J 8.5 Hz, 6H), 1.80 (s, 4H), 1.71 (d, J 10.9 Hz, 2H), 1.28-1.10 (m, 3H), 0.59 (d, J 6.4 Hz, 3H), 0.20 (d, J 6.2 Hz, 3H).

### Example 67: Preparation of Compound 88

### Step 1: (11R)-6-(2,6-Dimethylphenyl)-12-[(4-dimethylphosphorylphenyl)methyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 88)

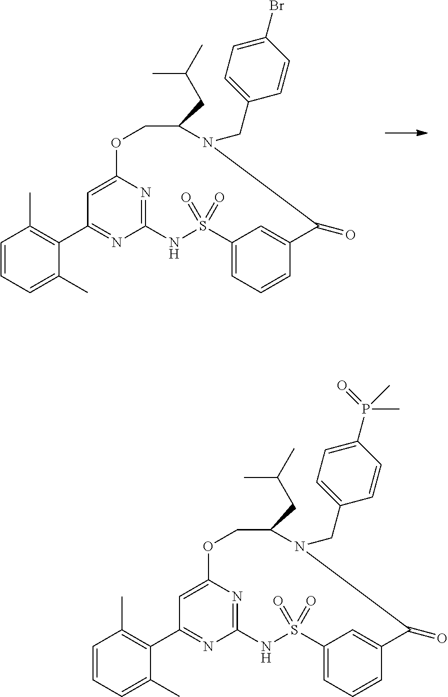 [SMILES: Cc1cccc(C)c1-c1cc2nc(n1)NS(=O)(=O)c1cccc(c1)C(=O)N(Cc1ccc(Br)cc1)[C@H](CC(C)C)CO2 ; Cc1cccc(C)c1-c1cc2nc(n1)NS(=O)(=O)c1cccc(c1)C(=O)N(Cc1ccc(P(C)(C)=O)cc1)[C@H](CC(C)C)CO2]

A heterogeneous solution of (11R)-12-[(4-bromophenyl)methyl]-6-(2,6-dimethylphenyl)-11-isobutyl-2,2-dioxo-9-oxa-2λ⁶-thia-3, 5,12,19-tetrazatricyclo[12.3.1.14,8]nonadeca-1(18),4(19),5,7,14,16-hexaen-13-one (20 mg, 0.03079 mmol), iodocopper (0.58 mg, 0.003045 mmol), cesium carbonate (30 mg, 0.09208 mmol), and methylphosphonoylmethane (7.2 mg, 0.09225 mmol) in toluene (100 μL) and DMF (100 μL) was microwaved in a sealed vessel to 120° C. for 40 min. The sample was purified by reverse phase HPLC (Phenomenex Luna C₁₈ column (75×30 mm, 5 μm particle size), gradient: 1-99% acetonitrile in water (5 mM HCl) over 15.0 minutes) which afforded (11R)-6-(2,6-dimethylphenyl)-12-[(4-dimethylphosphorylphenyl)methyl]-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (5.3 mg, 27%) as a white solid. ESI-MS m/z calc. 646.23785, found 647.1 (M+1)⁺; Retention time: 1.49 minutes; LC method A.

### Example 68: Preparation of Compound 89

### Step 1: (11R)-12-(7-Azaspiro[3.5]nonan-2-ylmethyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one

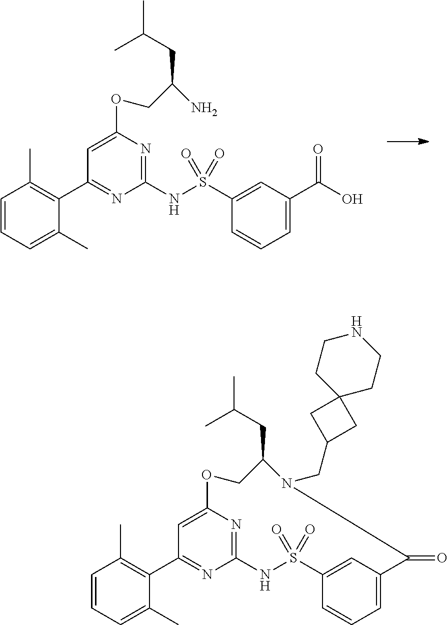 [SMILES: Cc1cccc(C)c1-c1cc(OC[C@H](N)CC(C)C)nc(NS(=O)(=O)c2cccc(C(=O)O)c2)n1 ; Cc1cccc(C)c1-c1cc2nc(n1)NS(=O)(=O)c1cccc(c1)C(=O)N(CC1CC3(CCNCC3)C1)[C@H](CC(C)C)CO2]

3-[[4-[(2R)-2-Amino-4-methyl-pentoxy]-6-(2,6-dimethylphenyl)pyrimidin-2-yl]sulfamoyl]benzoic acid (hydrochloride salt) (250 mg, 0.4672 mmol) and tert-butyl 2-formyl-7-azaspiro[3.5]nonane-7-carboxylate (180 mg, 0.7105 mmol) were combined in dichloromethane (1 mL) at room temperature. Sodium triacetoxyborohydride (200 mg, 0.9437 mmol) was added and the reaction mixture became homogenous after several minutes. After 30 minutes at room temperature The reaction mixture was diluted with 30 mL dichloromethane and washed with 30 mL 0.5M HCl. The aqueous layer was extracted an additional 3×25 mL dichloromethane, then the organic layers were combined, washed with brine, dried over sodium sulfate and concentrated. the resulting crude material (containing substantial double reductive amination side product) was used in the next step without purification. ESI-MS m/z calc. 735.3666, found 736.6 (M+1)⁺; Retention time: 0.57 minutes; (LC method J with a 1 min gradient).

The crude product from above was combined with HATU (267 mg, 0.7022 mmol) in DMF (50 mL) and DIPEA (400 μL, 2.296 mmol) was added. The reaction was stirred for 2 hours at room temperature, then poured into a round bottom flask containing 200 mL ethyl acetate and 200 mL 0.5 M HCl. The layers were separated, and the aqueous layer was extracted with an additional 100 mL ethyl acetate. The combined organics were washed 5×50 mL with water, followed by brine, and dried over sodium sulfate. After concentrating, the crude material was purified by chromatography on silica gel eluting with 0-10% methanol in dichloromethane (compound elutes around 5%) to give a foaming solid tert-butyl 2-[[(11R)-6-(2,6-dimethylphenyl)-11-isobutyl-2,2,13-trioxo-9-oxa-2λ⁶-thia-3, 5,12,19-tetrazatricyclo[12.3.1.14,8]nonadeca-1(18),4(19),5,7,14,16-hexaen-12-yl]methyl]-7-azaspiro[3.5]nonane-7-carboxylate (110 mg, 33%) ESI-MS m/z calc. 717.356, found 718.9 (M+1)⁺; Retention time: 0.81 minutes; (LC method D).

The material was dissolved in dichloromethane (4 mL) and HCl (2.5 mL of 4 M, 10.00 mmol) was added. The reaction mixture was stirred at room temperature for 30 minutes, then concentrated under reduced pressure. The product was re-concentrated from hexanes 3×, until a solid was obtained, (11R)-12-(7-azaspiro[3.5]nonan-2-ylmethyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (98 mg, 32%) ESI-MS m/z calc. 617.3036, found 618.8 (M+1)⁺; Retention time: 0.52 minutes; (LC method D).

### Step 2: (11R)-6-(2,6-Dimethylphenyl)-11-isobutyl-12-[(7-methyl-7-azaspiro[3.5]nonan-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 89)

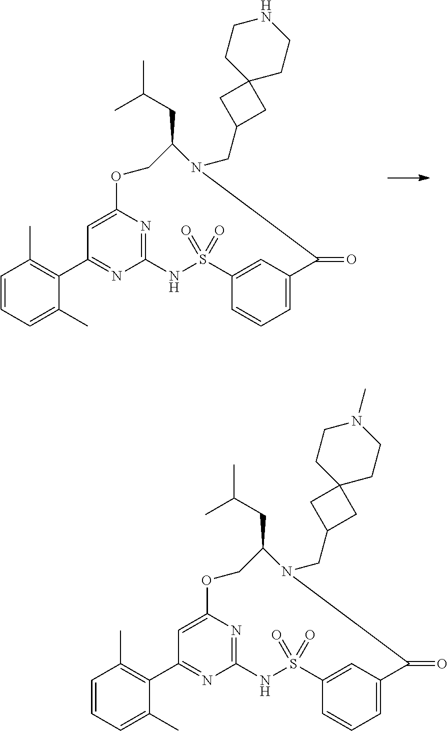 [SMILES: Cc1cccc(C)c1-c1cc2nc(n1)NS(=O)(=O)c1cccc(c1)C(=O)N(CC1CC3(CCNCC3)C1)[C@H](CC(C)C)CO2 ; Cc1cccc(C)c1-c1cc2nc(n1)NS(=O)(=O)c1cccc(c1)C(=O)N(CC1CC3(CCN(C)CC3)C1)[C@H](CC(C)C)CO2]

(11R)-12-(7-Azaspiro[3.5]nonan-2-ylmethyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (75 mg, 0.1214 mmol) was dissolved in formic acid (0.3 mL) and combined with aqueous formaldehyde (0.3 mL, 10.89 mmol) and heated to 90° C. for 20 hours in a screwcap vial. The reaction mixture was then diluted with methanol, filtered, then purified by reverse phase HPLC (1-99% ACN in water, HCl modifier, 15 minutes) to give as a white powder, (11R)-6-(2,6-dimethylphenyl)-11-isobutyl-12-[(7-methyl-7-azaspiro[3.5]nonan-2-yl)methyl]-2,2-dioxo-9-oxa-2λ⁶-thia-3, 5,12,19-tetrazatricyclo[12.3.1.14,8]nonadeca-1(18),4(19),5,7,14,16-hexaen-13-one (hydrochloride salt) (39 mg, 48%). ESI-MS m/z calc. 631.3192, found 632.8 (M+1)⁺; Retention time: 1.33 minutes; LC method A. ¹H NMR (400 MHz, DMSO) δ 9.88 (s, 1H), 8.36 (s, 1H), 7.92 (d, J 6.8 Hz, 1H), 7.67 (d, J 6.4 Hz, 2H), 7.27 (t, J 7.6 Hz, 1H), 7.13 (d, J 7.7 Hz, 2H), 6.42 (d, J 6.0 Hz, 1H), 5.06 (dt, J 10.1, 4.1 Hz, 1H), 4.34 (dt, J 21.4, 11.0 Hz, 1H), 3.94-3.77 (m, 2H), 3.68 (dd, J 13.6, 5.1 Hz, 2H), 3.25 (t, J 13.0 Hz, 2H), 3.11 (dd, J 13.7, 8.4 Hz, 1H), 2.96 (q, J 10.8 Hz, 1H), 2.88-2.77 (m, 1H), 2.73-2.68 (m, 3H), 2.67-2.59 (m, 1H), 2.14-1.96 (m, 6H), 1.94 (d, J 4.2 Hz, 1H), 1.88 (dq, J 10.7, 3.3 Hz, 1H), 1.77 (d, J 9.5 Hz, 2H), 1.65 (ddd, J 24.8, 14.1, 10.2 Hz, 4H), 1.31-1.10 (m, 2H), 0.74 (d, J 6.3 Hz, 3H), 0.24 (d, J 5.9 Hz, 3H).

### Example 69: Preparation of Compound 90

### Step 1: (11R)-12-[(7-Acetyl-7-azaspiro[3.5]nonan-2-yl)methyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 90)

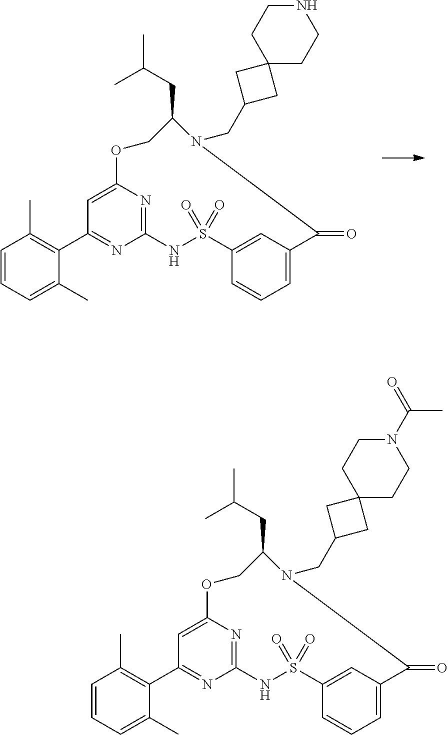 [SMILES: Cc1cccc(C)c1-c1cc2nc(n1)NS(=O)(=O)c1cccc(c1)C(=O)N(CC1CC3(CCNCC3)C1)[C@H](CC(C)C)CO2 ; CC(=O)N1CCC2(CC1)CC(CN1C(=O)c3cccc(c3)S(=O)(=O)Nc3nc(cc(-c4c(C)cccc4C)n3)OC[C@H]1CC(C)C)C2]

(11R)-12-(7-azaspiro[3.5]nonan-2-ylmethyl)-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (17 mg, 0.02598 mmol) was combined in dichloromethane (250 μL) with acetic anhydride (6 μL, 0.06359 mmol), and triethylamine (20 μL, 0.1435 mmol). The reaction mixture was concentrated then diluted with 1:1 DMSO/methanol, filtered, and purified by reverse phase HPLC (1-99% ACN in water 15 min run, HCl modifier) to give as a white powder, (11R)-12-[(7-acetyl-7-azaspiro[3.5]nonan-2-yl)methyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (10 mg, 58%). ESI-MS m/z calc. 659.31415, found 660.8 (M+1)⁺; Retention time: 1.73 minutes (LC method A).

### Example 70: Preparation of Compound 91

### Step 1: (11R)-6-(2,6-Dimethylphenyl)-11-isobutyl-12-[2-(methylamino)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one

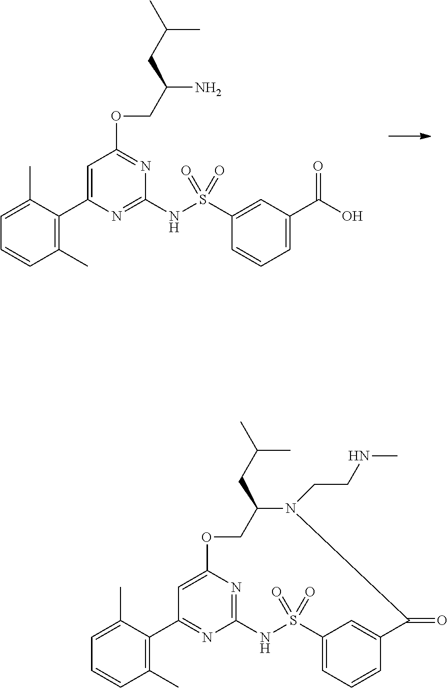 [SMILES: Cc1cccc(C)c1-c1cc(OC[C@H](N)CC(C)C)nc(NS(=O)(=O)c2cccc(C(=O)O)c2)n1 ; CNCCN1C(=O)c2cccc(c2)S(=O)(=O)Nc2nc(cc(-c3c(C)cccc3C)n2)OC[C@H]1CC(C)C]

3-[[4-[(2R)-2-Amino-4-methyl-pentoxy]-6-(2,6-dimethylphenyl)pyrimidin-2-yl]sulfamoyl]benzoic acid (hydrochloride salt) (250 mg, 0.4672 mmol) and tert-butyl N-methyl-N-(2-oxoethyl)carbamate (100 mg, 0.5773 mmol) combined in dichloromethane (934.5 μL) and stirred at room temperature for 5 minutes. Sodium triacetoxyborohydride (200 mg, 0.9437 mmol) was then added and stirring was continued for an additional hour, followed by a second addition of sodium triacetoxyborohydride (200 mg, 0.9437 mmol) and an additional hour of reaction time. The reaction mixture was then partitioned between 1M HCl and ethyl acetate and the layers were separated. The aqueous layer was extracted 3 with ethyl acetate and the combined organics were washed with brine, dried over sodium sulfate and concentrated. The crude material was purified by reverse phase chromatography (Cis column, 1-99% ACN in water, HCl modifier). Fractions containing product were concentrated and used in the next step. The product was combined with HATU (approximately 266.5 mg, 0.7008 mmol) in DMF and DIPEA (approximately 60.38 mg, 81.37 μL, 0.4672 mmol) was added. The reaction mixture was stirred at room temperature for 2 hours, then was partitioned between 1 M HCl and ethyl acetate. The layers were separated and the aqueous was extracted an additional three times with ethyl acetate. The combined organics were washed with brine and dried over sodium sulfate. After concentrating the crude product was purified by chromatography on silica gel eluting with a gradient of 0-10% methanol in dichloromethane. The fractions containing product were concentrated to give foaming solids that were used in the next step. The product was dissolved in dichloromethane, and HCl in dioxane (approximately 584.0 μL of 4 M, 2.336 mmol) was added. The reaction mixture was stirred for 30 minutes at room temperature. The reaction mixture was then evaporated. hexanes were added, and the reaction mixture was evaporated a second time to give as a solid, (11R)-6-(2,6-dimethylphenyl)-11-isobutyl-12-[2-(methylamino)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (102 mg, 38%) ESI-MS m/z calc. 537.24097, found 538.4 (M+1)⁺; Retention time: 0.47 minutes; LC method D.

### Step 2: Isopropyl N-[2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]ethyl]-N-methyl-carbamate (Compound 91)

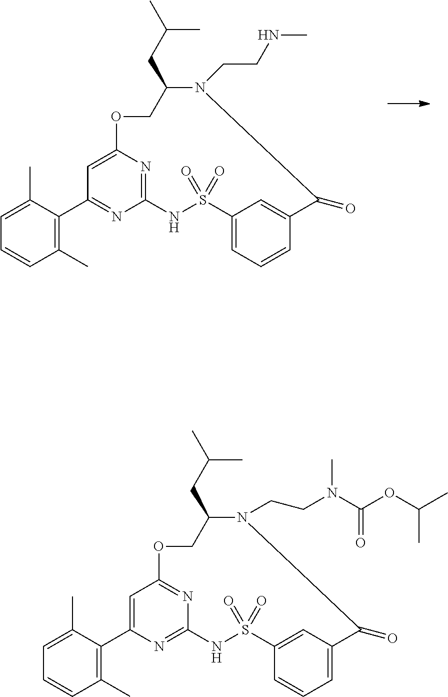 [SMILES: CNCCN1C(=O)c2cccc(c2)S(=O)(=O)Nc2nc(cc(-c3c(C)cccc3C)n2)OC[C@H]1CC(C)C ; Cc1cccc(C)c1-c1cc2nc(n1)NS(=O)(=O)c1cccc(c1)C(=O)N(CCN(C)C(=O)OC(C)C)[C@H](CC(C)C)CO2]

The (11R)-6-(2,6-dimethylphenyl)-11-isobutyl-12-[2-(methylamino)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (12 mg, 0.02090 mmol) dissolved in DCM with DIPEA (approximately 13.51 mg, 18.21 μL, 0.1045 mmol). Isopropyl chloroformate (approximately 15.68 μL of 2 M, 0.03135 mmol) was added, and the reaction was stirred for 30 minutes at room temperature. The reaction was then quenched with 2 drops of 1M HCl, partially concentrated, dissolved in 1:1 methanol/DMSO, filtered and purified by reverse phase HPLC (1-99% ACN in water, HCl modifier, 15 min run) to give the corresponding isopropyl N-[2-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]ethyl]-N-methyl-carbamate (6.9 mg, 52%). ESI-MS m z calc. 623.2778, found 624.3 (M+1)⁺; Retention time: 1.83 minutes; LC method A.

### Example 71: Preparation of Compound 92

### Step 1: 3-[[4-(2,6-Dimethylphenyl)-6-[(2R)-2-[(4-methoxy-4-oxo-butyl)amino]-4-methyl-pentoxy]pyrimidin-2-yl]sulfamoyl]benzoic acid

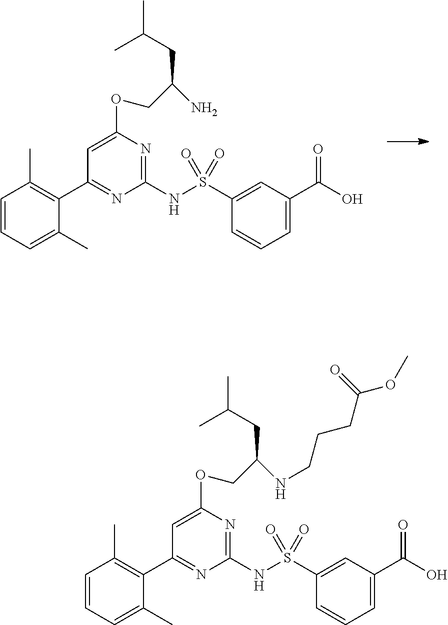 [SMILES: Cc1cccc(C)c1-c1cc(OC[C@H](N)CC(C)C)nc(NS(=O)(=O)c2cccc(C(=O)O)c2)n1 ; COC(=O)CCCN[C@@H](COc1cc(-c2c(C)cccc2C)nc(NS(=O)(=O)c2cccc(C(=O)O)c2)n1)CC(C)C]

3-[[4-[(2R)-2-Amino-4-methyl-pentoxy]-6-(2,6-dimethylphenyl)pyrimidin-2-yl]sulfamoyl]benzoic acid (hydrochloride salt) (293 mg, 0.5147 mmol), methyl 4-oxobutanoate (77.2 mg, 0.6649 mmol), and sodium triacetoxyborohydride (321 mg, 1.515 mmol) were combined in DCM (2 mL) and stirred at room temperature for 2 h. The reaction was partitioned between ethyl acetate and a 1 M HCl solution. The organics were separated, washed with brine, dried over sodium sulfate and evaporated. The crude material was purified by reverse-phase HPLC utilizing a gradient of 1-99% acetonitrile in 5 mM aqueous HCl to yield 3-[[4-(2,6-dimethylphenyl)-6-[(2R)-2-[(4-methoxy-4-oxo-butyl)amino]-4-methyl-pentoxy]pyrimidin-2-yl]sulfamoyl]benzoic acid (106 mg, 34%). ESI-MS m/z calc. 598.2461, found 599.4 (M+1)⁺; Retention time: 0.47 minutes; LC method D.

### Step 2: 4-[(11R)-6-(2,6-Dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]butanoic acid

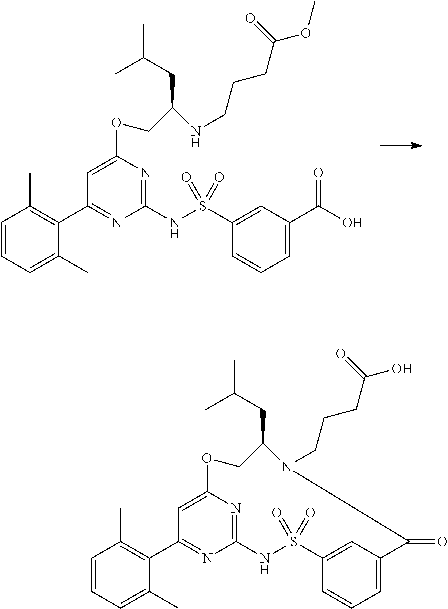 [SMILES: COC(=O)CCCN[C@@H](COc1cc(-c2c(C)cccc2C)nc(NS(=O)(=O)c2cccc(C(=O)O)c2)n1)CC(C)C ; Cc1cccc(C)c1-c1cc2nc(n1)NS(=O)(=O)c1cccc(c1)C(=O)N(CCCC(=O)O)[C@H](CC(C)C)CO2]

3-[[4-(2,6-Dimethylphenyl)-6-[(2R)-2-[(4-methoxy-4-oxo-butyl)amino]-4-methyl-pentoxy]pyrimidin-2-yl]sulfamoyl]benzoic acid (106 mg, 0.1770 mmol) was dissolved in DMF (4 mL). HATU (84.7 mg, 0.2228 mmol) was added, followed by triethylamine (100 μL, 0.7175 mmol) and the reaction was stirred for 30 min at room temperature. The reaction was partitioned between ethyl acetate and a 1M HCl solution. The organics were separated, washed with brine, dried over sodium sulfate and evaporated. The crude material was used directly in the next step. The product was dissolved in a mixture of THE (2 mL): NaOH (2 mL of 1 M, 2.000 mmol) and the mixture was stirred at room temperature for 2 h. The reaction mixture was partitioned between ethyl acetate and a 1M HCl solution. The organics were separated, washed with brine, dried over sodium sulfate and evaporated to give 4-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]butanoic acid (100 mg, 100%). ESI-MS m/z calc. 566.2199, found 567.4 (M+1)⁺; Retention time: 0.59 minutes; LC method D.

### Step 3: 4-[(11R)-6-(2,6-Dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-N,N-dimethyl-butanamide (Compound 92)

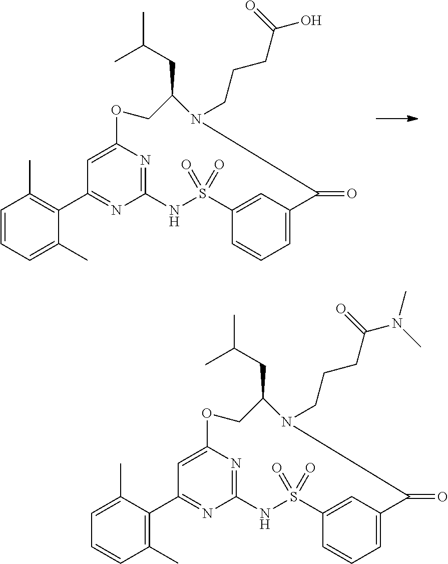 [SMILES: Cc1cccc(C)c1-c1cc2nc(n1)NS(=O)(=O)c1cccc(c1)C(=O)N(CCCC(=O)O)[C@H](CC(C)C)CO2 ; Cc1cccc(C)c1-c1cc2nc(n1)NS(=O)(=O)c1cccc(c1)C(=O)N(CCCC(=O)N(C)C)[C@H](CC(C)C)CO2]

4-[(11R)-6-(2,6-Dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]butanoic acid (15 mg, 0.02647 mmol) was combined with the N-methylmethanamine (hydrochloride salt) (7 mg, 0.08584 mmol) and HATU (20 mg, 0.05260 mmol) in DMF (0.5 mL). DIPEA (40 μL, 0.2296 mmol) was added and the reaction mixture was stirred at room temperature for 30 minutes. The reaction mixture was then filtered and purified by reverse phase HPLC (1-70% ACN in water, HCl modifier, 15 min run) to give as a white solid 4-[(11R)-6-(2,6-dimethylphenyl)-11-isobutyl-2,2,13-trioxo-9-oxa-2λ⁶-thia-3,5,12,19-tetrazatricyclo[12.3.1.14,8]nonadeca-1(18),4(19),5,7,14,16-hexaen-12-yl]-N,N-dimethyl-butanamide (7.2 mg, 45%). ESI-MS m z calc. 593.2672, found 594.3 (M+1)⁺; Retention time: 1.54 minutes; LC method A.

### Example 72: Preparation of Compound 93, Compound 94, and Compound 95

### Step 1: 3-[[4-(2,6-Dimethylphenyl)-6-[(2R)-2-[(6-methoxycarbonyl-3-pyridyl)methylamino]-4-methyl-pentoxy]pyrimidin-2-yl]sulfamoyl]benzoic acid

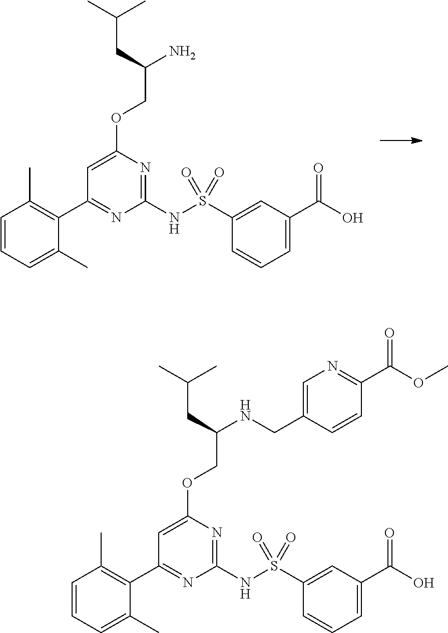 [SMILES: Cc1cccc(C)c1-c1cc(OC[C@H](N)CC(C)C)nc(NS(=O)(=O)c2cccc(C(=O)O)c2)n1 ; COC(=O)c1ccc(CN[C@@H](COc2cc(-c3c(C)cccc3C)nc(NS(=O)(=O)c3cccc(C(=O)O)c3)n2)CC(C)C)cn1]

In a flask containing 3-[[4-[(2R)-2-amino-4-methyl-pentoxy]-6-(2,6-dimethylphenyl)pyrimidin-2-yl]sulfamoyl]benzoic acid (hydrochloride salt) (513 mg, 0.7050 mmol) in DCM (5 mL) was added methyl 5-formylpyridine-2-carboxylate (141 mg, 0.8538 mmol) and sodium triacetoxyborohydride (484 mg, 2.284 mmol). The solution was then stirred at rt for 3 h. A small quantity of 1N HCl was added. The solvent was evaporated and the crude mixture was purified by reverse phase chromatography using a 0-100% gradient of MeCN in water (formic acid modifier) to give 3-[[4-(2,6-dimethylphenyl)-6-[(2R)-2-[(6-methoxycarbonyl-3-pyridyl)methylamino]-4-methyl-pentoxy]pyrimidin-2-yl]sulfamoyl]benzoic acid (317 mg, 63%). ESI-MS m/z calc. 647.2414, found 648.3 (M+1)⁺; Retention time: 0.7 minutes.

### Step 2: Methyl 5-{[(11R)-6-(2,6-Dimethylphenyl)-11-(2-methylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]methyl}pyridine-2-carboxylate (Compound 95)

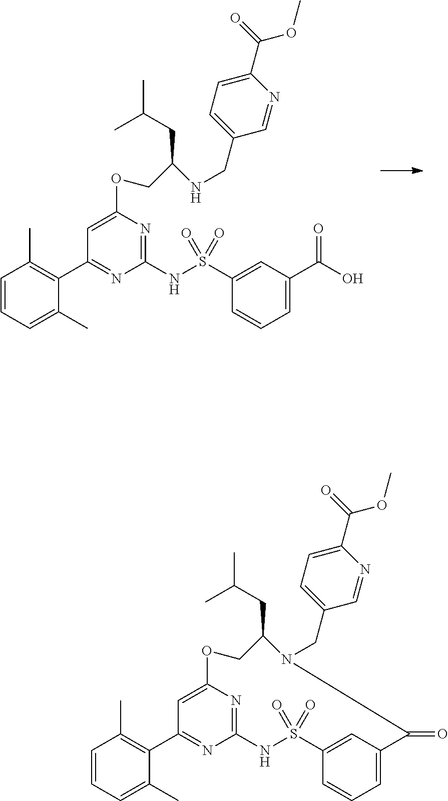 [SMILES: COC(=O)c1ccc(CN[C@@H](COc2cc(-c3c(C)cccc3C)nc(NS(=O)(=O)c3cccc(C(=O)O)c3)n2)CC(C)C)cn1 ; COC(=O)c1ccc(CN2C(=O)c3cccc(c3)S(=O)(=O)Nc3nc(cc(-c4c(C)cccc4C)n3)OC[C@H]2CC(C)C)cn1]

A solution of 3-[[4-(2,6-dimethylphenyl)-6-[(2R)-2-[(6-methoxycarbonyl-3-pyridyl)methylamino]-4-methyl-pentoxy]pyrimidin-2-yl]sulfamoyl]benzoic acid (hydrochloride salt) (0.16 g, 0.2338 mmol), HATU (0.14 g, 0.3682 mmol), and triethylamine (0.14 mL, 1.004 mmol) in DMF (12 mL) was stirred for 17 hours. The reaction was acidified with 1 M citric acid, diluted with water, and extracted with ethyl acetate. The combined extracts were washed with brine and water, dried over sodium sulfate, and evaporated under vacuum. The residue was purified by silica gel column chromatography with 0-5% methanol in dichloromethane to give 0.10 g mostly clean product. A small portion of this was purified by reverse-phase HPLC (1%-99% acetonitrile/water (5 mM HCl)) to give methyl 5-{[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]methyl}pyridine-2-carboxylate (0.10 g, 67%). ESI-MS m z calc. 629.23083, found 630.4 (M+1)⁺; Retention time: 1.57 minutes; LC method A.

### Step 3: 5-{[(11R)-6-(2,6-Dimethylphenyl)-11-(2-methylpropyl)-2,2,13-trioxo-9-oxa-2)⁶-thia-3,5,12,19-tetraazatricyclo[12.3.1.14,8]nonadeca-1(18),4(19),5,7,14,16-hexaen-12-yl]methyl}pyridine-2-carboxylic acid (Compound 94)

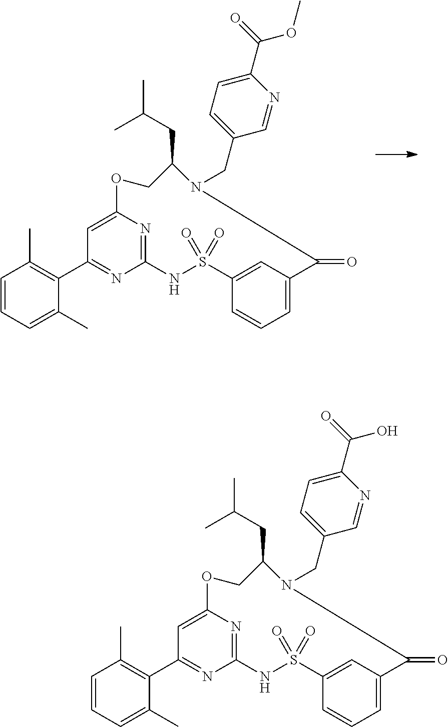 [SMILES: COC(=O)c1ccc(CN2C(=O)c3cccc(c3)S(=O)(=O)Nc3nc(cc(-c4c(C)cccc4C)n3)OC[C@H]2CC(C)C)cn1 ; Cc1cccc(C)c1-c1cc2nc(n1)NS(=O)(=O)c1cccc(c1)C(=O)N(Cc1ccc(C(=O)O)nc1)[C@H](CC(C)C)CO2]

A solution of methyl 5-{[(1 1R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]methyl}pyridine-2-carboxylate (83 mg, 0.1305 mmol) and lithium hydroxide hydrate (27 mg, 0.6434 mmol) in THE (0.7 mL) and water (0.7 mL) was stirred for three hours. The reaction was quenched with 0.65 mL of 1 M hydrochloric acid, diluted with water, and extracted with ethyl acetate. The combined extracts were dried over sodium sulfate and evaporated under vacuum to give 5-{[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]methyl}pyridine-2-carboxylic acid (80 mg, 100%) as a colorless solid. ESI-MS m/z calc. 615.21515, found 616.4 (M+1)⁺; Retention time: 0.56 minutes; LC method D.

### Step 4: 5-{[(11R)-6-(2,6-Dimethylphenyl)-11-(2-methylpropyl)-2,2,13-trioxo-9-oxa-2)⁶-thia-3,5,12,19-tetraazatricyclo[12.3.1.14,8]nonadeca-1(18),4(19),5,7,14,16-hexaen-12-yl]methyl}-N-methylpyridine-2-carboxamide (Compound 93)

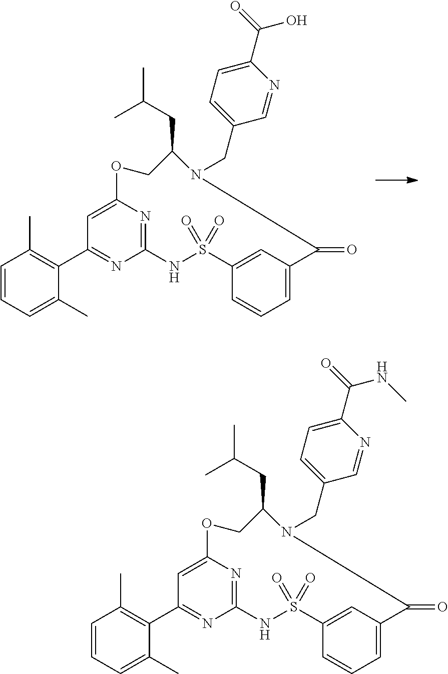 [SMILES: Cc1cccc(C)c1-c1cc2nc(n1)NS(=O)(=O)c1cccc(c1)C(=O)N(Cc1ccc(C(=O)O)nc1)[C@H](CC(C)C)CO2 ; CNC(=O)c1ccc(CN2C(=O)c3cccc(c3)S(=O)(=O)Nc3nc(cc(-c4c(C)cccc4C)n3)OC[C@H]2CC(C)C)cn1]

A solution of 5-{[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]methyl}pyridine-2-carboxylic acid (41 mg, 0.06659 mmol), HATU (39 mg, 0.1026 mmol), and methylamine in methanol (0.17 mL of 2 M, 0.3400 mmol) in DMF (0.4 mL) was stirred for five hours. More methylamine in methanol (0.2 mL of 2 M, 0.4000 mmol) and HATU (27 mg, 0.07101 mmol) were added. The reaction was stirred for three days and purified by reverse-phase HPLC (1%-99% acetonitrile/water (5 mM HCl)) to give 5-{[(11R)-6-(2,6-dimethylphenyl)-11-(2-methylpropyl)-2,2,13-trioxo-9-oxa-2λ⁶-thia-3,5,12,19-tetraazatricyclo[12.3.1.14,8]nonadeca-1(18),4(19),5,7,14,16-hexaen-12-yl]methyl}-N-methylpyridine-2-carboxamide (9.7 mg, 23%) ESI-MS m/z calc. 628.24677, found 629.4 (M+1)⁺; Retention time: 1.56 minutes (LC method A). ¹H NMR (400 MHz, DMSO-d₆) δ 8.79 (dd, J 2.3, 0.9 Hz, 1H), 8.52 (d, J 1.8 Hz, 1H), 8.12-8.01 (m, 2H), 7.95 (d, J 7.7 Hz, 1H), 7.76 (d, J 7.5 Hz, 1H), 7.74-7.64 (m, 1H), 7.26 (t, J 7.6 Hz, 1H), 7.17-7.07 (m, 2H), 6.42 (s, 1H), 5.20 (dd, J 10.5, 4.4 Hz, 1H), 4.79 (s, 2H), 4.44 (t, J 11.1 Hz, 1H), 4.03-3.92 (m, 1H), 2.02 (s, 6H), 1.66 (t, J 11.5 Hz, 1H), 1.28-1.08 (m, 2H), 0.54 (d, J 5.8 Hz, 3H), 0.18 (d, J 5.6 Hz, 3H)

### Example 73: Preparation of Compound 96

### Step 1: 3-[[4-[(2R)-2-[3-[tert-Butyl(dimethyl)silyl]oxypropylamino]-4-methyl-pentoxy]-6-(2,6-dimethylphenyl)pyrimidin-2-yl]sulfamoyl]benzoic acid

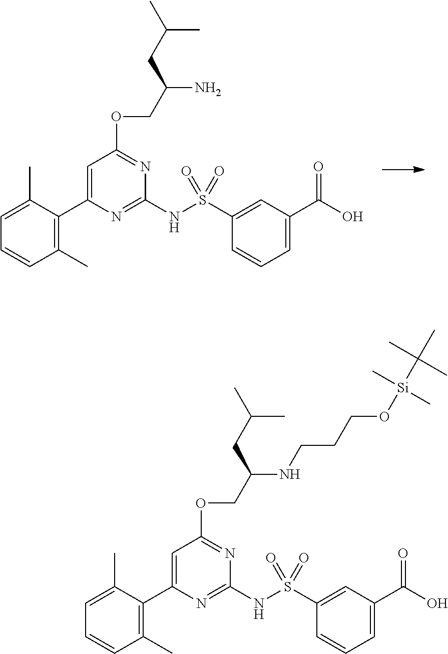 [SMILES: Cc1cccc(C)c1-c1cc(OC[C@H](N)CC(C)C)nc(NS(=O)(=O)c2cccc(C(=O)O)c2)n1 ; Cc1cccc(C)c1-c1cc(OC[C@@H](CC(C)C)NCCCO[Si](C)(C)C(C)(C)C)nc(NS(=O)(=O)c2cccc(C(=O)O)c2)n1]

A solution of 3-[[4-[(2R)-2-amino-4-methyl-pentoxy]-6-(2,6-dimethylphenyl)pyrimidin-2-yl]sulfamoyl]benzoic acid (hydrochloride salt) (0.32 g, 0.5981 mmol), 3-[tert-butyl(dimethyl)silyl]oxypropanal (0.13 g, 0.6902 mmol), and sodium triacetoxyborohydride (0.25 g, 1.180 mmol) in dichloromethane (3 mL) was stirred for two hours. Another three drops of aldehyde were added, and the reaction was stirred for an hour. Five more drops of aldehyde and 0.1 g sodium triacetoxyborohydride were added, and the reaction was stirred for two hours. It was acidified with 1 M citric acid, diluted with water, and extracted with ethyl acetate. The combined extracts were dried over sodium sulfate and evaporated under vacuum. The residue was purified by silica gel column chromatography with 0-10% methanol in dichloromethane to give 3-[[4-[(2R)-2-[3-[tert-butyl(dimethyl)silyl]oxypropylamino]-4-methyl-pentoxy]-6-(2,6-dimethylphenyl)pyrimidin-2-yl]sulfamoyl]benzoic acid (0.12 g, 30%). ESI-MS m/z calc. 670.322, found 671.5 (M+1)⁺; Retention time: 0.64 minutes; LC method D.

### Step 2: (11R)-12-{3-[(tert-Butyldimethylsilyl)oxy]propyl}-6-(2,6-dimethylphenyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione

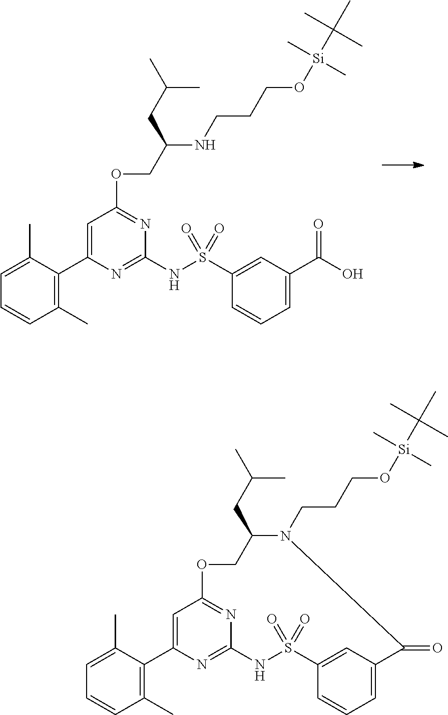 [SMILES: Cc1cccc(C)c1-c1cc(OC[C@@H](CC(C)C)NCCCO[Si](C)(C)C(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)N(CCCO[Si](C)(C)C(C)(C)C)[C@H](CC(C)C)CO2]

A solution of 3-[[4-[(2R)-2-[3-[tert-butyl(dimethyl)silyl]oxypropylamino]-4-methyl-pentoxy]-6-(2,6-dimethylphenyl)pyrimidin-2-yl]sulfamoyl]benzoic acid (0.12 g, 0.1789 mmol), 4-(6-cyano-2-methyl-7-oxo-4,8-dioxa-2,5-diazadec-5-en-3-ylidene)morpholin-4-ium hexafluorophosphate(V) (0.12 g, 0.2802 mmol), and DIEA (95 μL, 0.5454 mmol) in DMF (9 mL) was stirred for 22 hours. The reaction was diluted with water, acidified with 1 M citric acid, and extracted with ethyl acetate. The combined extracts were washed with brine, dried over sodium sulfate, and evaporated under vacuum. The residue was purified by silica gel column chromatography with 0-5% methanol in dichloromethane to give (11R)-12-{3-[(tert-butyldimethylsilyl)oxy]propyl}-6-(2,6-dimethylphenyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione (50 mg, 43%) as a light yellow oil. ESI-MS m/z calc. 652.31146, found 653.3 (M+1)⁺; Retention time: 0.92 minutes; (LC method A).

### Step 3: (11R)-6-(2,6-Dimethylphenyl)-12-(3-hydroxypropyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione (Compound 96)

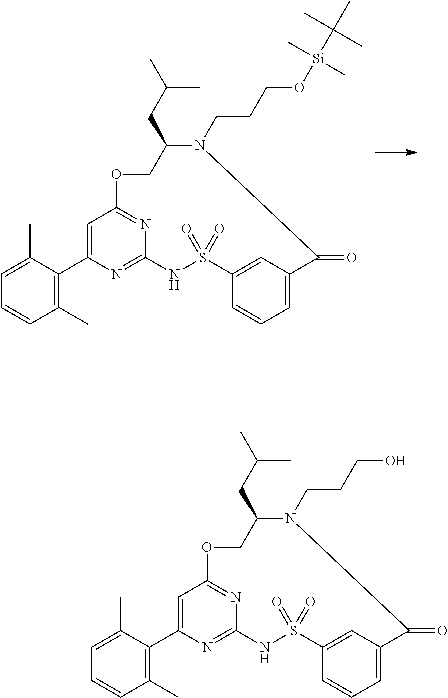 [SMILES: Cc1cccc(C)c1-c1cc2nc(n1)NS(=O)(=O)c1cccc(c1)C(=O)N(CCCO[Si](C)(C)C(C)(C)C)[C@H](CC(C)C)CO2 ; Cc1cccc(C)c1-c1cc2nc(n1)NS(=O)(=O)c1cccc(c1)C(=O)N(CCCO)[C@H](CC(C)C)CO2]

A solution of (11R)-12-{3-[(tert-butyldimethylsilyl)oxy]propyl}-6-(2,6-dimethylphenyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione (50 mg, 0.07658 mmol) in HCl in dioxane (3 mL of 4 M, 12.00 mmol) was stirred for 10 minutes. The solvent was removed under vacuum, and the residue was purified by reverse-phase HPLC (15%-75% acetonitrile/water (5 mM HCl)) to give (11R)-6-(2,6-dimethylphenyl)-12-(3-hydroxypropyl)-11-(2-methylpropyl)-9-oxa-2λ⁶-thia-3,5,12,19-tetraazatricyclo[12.3.1.14,8]nonadeca-1(18),4,6,8(19),14,16-hexaene-2,2,13-trione (hydrochloride salt) (17.3 mg, 39%) as a colorless solid. ESI-MS m/z calc. 538.225, found 539.3 (M+1)⁺; Retention time: 1.42 minutes; LC method A.

### Example 74: Preparation of Compound 97

### Step 1: tert-Butyl N-{3-[benzyl(methyl)amino]-2-hydroxypropyl}carbamate

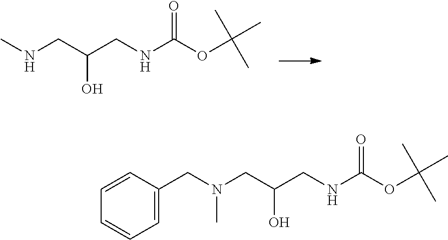 [SMILES: CNCC(O)CNC(=O)OC(C)(C)C ; CN(Cc1ccccc1)CC(O)CNC(=O)OC(C)(C)C]

In a 100-mL flask, tert-butyl N-[2-hydroxy-3-(methylamino)propyl]carbamate (1 g, 4.896 mmol) was dissolved in DMF (40 mL), followed by benzaldehyde (600 μL, 5.903 mmol) and stirred for 30 minutes. Then the mixture was cooled to 0° C. and to it was added sodium triacetoxyborohydride (3.15 g, 14.86 mmol) and the solution was stirred for 20 hours allowing to warm to RT. The mixture was diluted with water (20 mL),extracted with ethyl acetate, washed with saturated sodium bicarbonate (20 mL), and brine (20 mL), dried over sodium sulfate and concentrated. The residue was purified by silica gel chromatography (40 gram column) using a gradient from 100% dichloromethane to 15% methanol in dichloromethane to isolate as a clear oil tert-butyl N-{3-[benzyl(methyl)amino]-2-hydroxypropyl}carbamate (1.43 g, 99%). ESI-MS m/z calc. 294.19434, found 295.2 (M+1)⁺; Retention time: 0.81 minutes; LC method A. ¹H NMR (400 MHz, DMSO-d₆) δ 7.38-7.27 (m, 4H), 7.23 (ddd, J 8.8, 5.3, 3.6 Hz, 1H), 6.61 (t, J 5.7 Hz, 1H), 4.57 (d, J 4.6 Hz, 1H), 3.66 (dt, J 6.6, 4.8 Hz, 1H), 3.59-3.40 (m, 2H), 3.10 (dt, J 13.4, 5.5 Hz, 1H), 2.88-2.75 (m, 1H), 2.41-2.19 (m, 2H), 2.12 (s, 3H), 1.37 (s, 9H).

### Step 2: 3-{[4-({1-[Benzyl(methyl)amino]-3-{[(tert-butoxy)carbonyl]amino}propan-2-yl}oxy)-6-(2,6-dimethylphenyl)pyrimidin-2-yl]sulfamoyl}benzoic acid

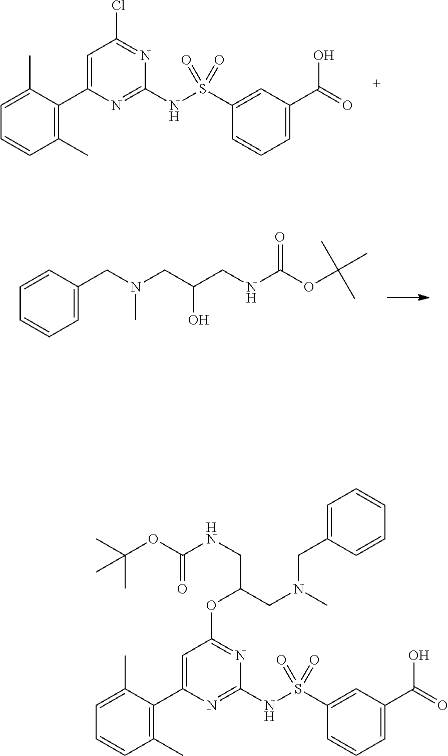 [SMILES: Cc1cccc(C)c1-c1cc(Cl)nc(NS(=O)(=O)c2cccc(C(=O)O)c2)n1 ; CN(Cc1ccccc1)CC(O)CNC(=O)OC(C)(C)C ; Cc1cccc(C)c1-c1cc(OC(CNC(=O)OC(C)(C)C)CN(C)Cc2ccccc2)nc(NS(=O)(=O)c2cccc(C(=O)O)c2)n1]

In a 250 mL flask, 3-[[4-chloro-6-(2,6-dimethylphenyl)pyrimidin-2-yl]sulfamoyl]benzoic acid (1.75 g, 4.188 mmol), tert-butyl N-{3-[benzyl(methyl)amino]-2-hydroxypropyl}carbamate (1.3 g, 4.416 mmol) and THE (45 mL) were mixed, to which KOtBu (2.82 g, 25.13 mmol) was added. This mixture was stirred at room temperature for 60 min. The mixture was quenched with 1 N HCl (to ˜pH 7) and extracted with ethyl acetate (3×75 mL). The combined organic extract was washed with water (50 mL) and saturated brine solution (100 mL), then dried over sodium sulfate, filtered, and evaporated in vacuo. This crude product was purified on silica gel chromatography (120 gram column) using a gradient from 100% dichloromethane to 20% methanol in dichloromethane to afford a white solid, 3-{[4-({1-[benzyl(methyl)amino]-3-{[(tert-butoxy)carbonyl]amino}propan-2-yl}oxy)-6-(2,6-dimethylphenyl)pyrimidin-2-yl]sulfamoyl}benzoic acid (580 mg, 20%). ESI-MS m/z calc. 675.27264, found 676.2 (M+1)⁺; Retention time: 1.28 minutes; LC method A. ¹H NMR (400 MHz, DMSO-d₆) δ 8.39 (t, J 1.8 Hz, 1H), 8.07 (t, J 6.7 Hz, 2H), 7.62 (t, J 7.8 Hz, 1H), 7.33-7.11 (m, 8H), 6.80 (s, 1H), 6.18 (s, 1H), 5.08 (s, 1H), 3.74-3.51 (m, 1H), 3.50-3.36 (m, 4H), 3.13-3.01 (m, 1H), 2.71-2.56 (m, 1H), 2.44 (s, 1H), 2.41-2.25 (m, 1H), 2.03 (s, 7H), 1.35 (d, J 18.9 Hz, 9H), 1.24 (s, 2H).

### Step 3: 3-({4-[(1-{[(tert-Butoxy)carbonyl]amino}-3-(methylamino)propan-2-yl)oxy]-6-(2,6-dimethylphenyl)pyrimidin-2-yl}sulfamoyl)benzoic acid

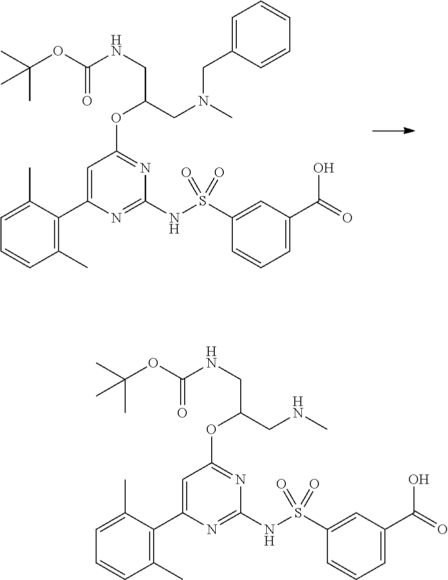 [SMILES: Cc1cccc(C)c1-c1cc(OC(CNC(=O)OC(C)(C)C)CN(C)Cc2ccccc2)nc(NS(=O)(=O)c2cccc(C(=O)O)c2)n1 ; CNCC(CNC(=O)OC(C)(C)C)Oc1cc(-c2c(C)cccc2C)nc(NS(=O)(=O)c2cccc(C(=O)O)c2)n1]

A mixture of 3-{[4-({1-[benzyl(methyl)amino]-3-{[(tert-butoxy)carbonyl]amino}propan-2-yl}oxy)-6-(2,6-dimethylphenyl)pyrimidin-2-yl]sulfamoyl}benzoic acid (620 mg, 0.9174 mmol) in MeOH (15.0 mL) was purged with nitrogen followed by Pd/C (98.5 mg of 10% w/w, 0.09256 mmol) (10% on carbon) followed by hydrogen then stirred under hydrogen (balloon) for 90 minutes. The reaction mixture was filtered over Celite and washed with excess methanol and then concentrated to afford a white solid 3-({4-[(1-{[(tert-butoxy)carbonyl]amino}-3-(methylamino)propan-2-yl)oxy]-6-(2,6-dimethylphenyl)pyrimidin-2-yl}sulfamoyl)benzoic acid (536 mg, 100%) ESI-MS m/z calc. 585.2257, found 586.2 (M+1)⁺; Retention time: 1.1 minutes (LC method A).

### Step 4: tert-Butyl N-[[6-(2,6-dimethylphenyl)-12-methyl-2,2,13-trioxo-9-oxa-2λ⁶-thia-3,5,12,19-tetrazatricyclo[12.3.1.14,8]nonadeca-1(18),4(19),5,7,14,16-hexaen-10-yl]methyl]carbamate (Compound 97)

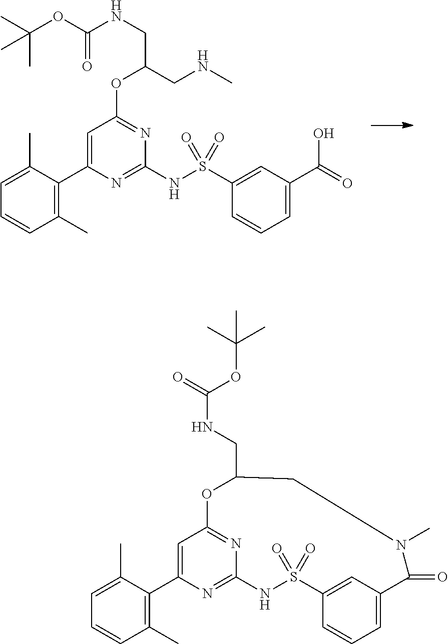 [SMILES: CNCC(CNC(=O)OC(C)(C)C)Oc1cc(-c2c(C)cccc2C)nc(NS(=O)(=O)c2cccc(C(=O)O)c2)n1 ; Cc1cccc(C)c1-c1cc2nc(n1)NS(=O)(=O)c1cccc(c1)C(=O)N(C)CC(CNC(=O)OC(C)(C)C)O2]

In a 100-mL flask, 3-({4-[(1-{[(tert-butoxy)carbonyl]amino}-3-(methylamino)propan-2-yl)oxy]-6-(2,6-dimethylphenyl)pyrimidin-2-yl}sulfamoyl)benzoic acid (500 mg, 0.8537 mmol) was dissolved in DMF (22.5 mL), to which DIPEA (750 μL, 4.306 mmol) and HATU (390 mg, 1.026 mmol) were added. After stirring at room temperature for 15 min. The mixture was diluted with aqueous 10% citric acid solution and extracted with ethyl acetate (3×25 mL). The combined organic extract was then washed with aqueous sodium bicarbonate (25 mL) and saturated brine solution (50 mL), then dried over sodium sulfate, filtered, and evaporated in vacuo. This crude product was purified on silica gel chromatography (40 gram column) using a gradient from 100% dichloromethane to 10% methanol in dichloromethane followed by a second silica gel chromatography (24 gram column) using a gradient from 100% hexanes to 70% ethyl acetate in hexanes to afford a white solid tert-butyl N-[[6-(2,6-dimethylphenyl)-12-methyl-2,2,13-trioxo-9-oxa-2λ⁶-thia-3,5,12,19-tetrazatricyclo[12.3.1.14,8]nonadeca-1(18),4(19),5,7,14,16-hexaen-10-yl]methyl]carbamate (205 mg, 42%)¹H NMR (400 MHz, DMSO-d₆) δ 12.96 (s, 1H), 8.43 (s, 1H), 7.88 (s, 1H), 7.63 (s, 2H), 7.26 (t, J 7.7 Hz, 1H), 7.18 (d, J 5.7 Hz, 1H), 7.13 (d, J 7.6 Hz, 2H), 6.12 (s, 1H), 5.52 (s, 1H), 3.71-3.58 (m, 1H), 3.58-3.42 (m, 1H), 3.42-3.35 (m, 1H), 3.21-3.12 (m, 1H), 3.08 (s, 3H), 2.06 (d, J=16.6 Hz, 6H), 1.38 (s, 9H).ESI-MS m/z calc. 567.21515, found 568.2 (M+1)⁺; Retention time: 1.41 minutes (LC method A).

### Example 75: Preparation of Compound 98

### Step 1: 10-(Aminomethyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one

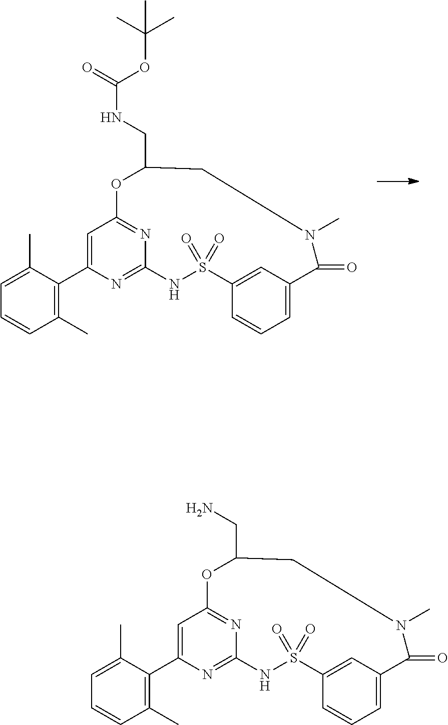 [SMILES: Cc1cccc(C)c1-c1cc2nc(n1)NS(=O)(=O)c1cccc(c1)C(=O)N(C)CC(CNC(=O)OC(C)(C)C)O2 ; Cc1cccc(C)c1-c1cc2nc(n1)NS(=O)(=O)c1cccc(c1)C(=O)N(C)CC(CN)O2]

tert-Butyl N-[[6-(2,6-dimethylphenyl)-12-methyl-2,2,13-trioxo-9-oxa-2λ⁶-thia-3,5,12,19-tetrazatricyclo[12.3.1.14,8]nonadeca-1(18),4(19),5,7,14,16-hexaen-10-yl]methyl]carbamate (71 mg, 0.1251 mmol) was dissolved in DCM (3.0 mL) and to the mixture was added HCl in dioxane (350 μL of 4 M, 1.400 mmol) and stirred at room temperature for 90 min. The reaction mixture was concentrated under reduced pressure to a white solid, which was then slurried in diethyl ether (2×10 mL). The solid was collected by vacuum filtration to provide as a white solid 10-(aminomethyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (63 mg, 100%). ESI-MS m/z calc. 467.16272, found 468.2 (M+1)⁺; Retention time: 0.76 minutes; LC method A.

### Step 2: 10-[[bis(3,3-Dimethylbutyl)amino]methyl]-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 98)

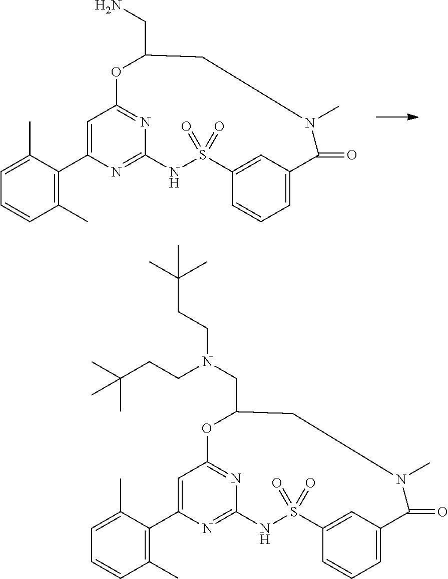 [SMILES: Cc1cccc(C)c1-c1cc2nc(n1)NS(=O)(=O)c1cccc(c1)C(=O)N(C)CC(CN)O2 ; Cc1cccc(C)c1-c1cc2nc(n1)NS(=O)(=O)c1cccc(c1)C(=O)N(C)CC(CN(CCC(C)(C)C)CCC(C)(C)C)O2]

In a 20-mL vial, 10-(aminomethyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (75 mg, 0.1488 mmol) was dissolved in DMF (3.0 mL), followed by 3,3-dimethylbutanal (75 μL, 0.5976 mmol) and stirred for 30 minutes. Then sodium triacetoxyborohydride (160 mg, 0.7549 mmol) was added and the solution was stirred for 1 hour at RT. The mixture was diluted with water, diluted with a saturated aqueous sodium bicarbonate solution and extracted with ethyl acetate (3×25 mL). The combined organic extract was washed then with saturated brine solution (50 mL), then dried over sodium sulfate, filtered, and evaporated in vacuo. The mixture was purified by reverse-phase preparative chromatography utilizing a C₁₈ column and a 10-70% gradient of acetonitrile in Water+5 mM HCl and concentrated to afford a white solid 10-[[bis(3,3-dimethylbutyl)amino]methyl]-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (39.57 mg, 39%) ESI-MS m/z calc. 635.3505, found 636.5 (M+1)⁺; Retention time: 1.41 minutes; LC method A. ¹H NMR (400 MHz, DMSO-d₆) δ 10.16 (s, 1H), 8.53 (s, 1H), 7.90 (s, 1H), 7.65 (s, 2H), 7.27 (t, J=7.7 Hz, 1H), 7.13 (d, J=7.6 Hz, 2H), 6.32 (s, 1H), 5.99 (s, 1H), 3.91 (t, J=12.1 Hz, 1H), 3.69 (dd, J=13.3, 6.6 Hz, 1H), 3.43 (s, 1H), 3.27 (dd, J=14.8, 10.1 Hz, 2H), 3.16 (d, J=9.6 Hz, 3H), 3.09 (s, 3H), 2.08 (s, 3H), 2.04 (s, 3H), 1.65 (dd, J=12.1, 6.3 Hz, 4H), 0.93 (s, 9H), 0.89 (s, 9H).

### Example 76: Preparation of Compound 99

### Step 1: tert-Butyl N-methyl-N-(oxiran-2-ylmethyl)carbamate

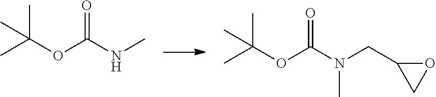

To a solution of tert-butyl N-methylcarbamate (5.0 g, 38.12 mmol) in DMF (50 mL) cooled to 0° C. was added NaH (60% in mineral oil) (1.68 g of 60% w/w, 42.00 mmol). After stirring the solution at 0° C. for 30 minutes and 1 hour at room temperature, a solution of 2-(bromomethyl)oxirane (3.1 mL, 36.23 mmol) in DMF (10 mL) was added dropwise to the light gray mixture. After stirring the solution at room temperature for 24 hours, the reaction mixture was diluted with ethyl acetate and quenched with water and brine. The ethyl acetate layer was then successively washed with water (2×), then brine, dried over sodium sulfate, filtered, and the solvent evaporated in vacuo. The resulting oil was purified by silica gel chromatography (120 gram column) using a gradient of 100% dichloromethane to 10% ethyl acetate in dichloromethane to afford a clear oil, tert-butyl N-methyl-N-(oxiran-2-ylmethyl)carbamate (2.2 g, 31%) ESI-MS m/z calc. 187.12085, found 188.2 (M+1)⁺; Retention time: 0.83 minutes; LC method A. ¹H NMR (400 MHz, Chloroform-d) δ 3.80-3.47 (m, 1H), 3.25-3.04 (m, 2H), 2.94 (s, 3H), 2.77 (s, 1H), 2.52 (dd, J 4.8, 2.2 Hz, 1H), 1.47 (s, 9H).

### Step 2: tert-Butyl N-(3-benzyloxy-2-hydroxy-propyl)-N-methyl-carbamate

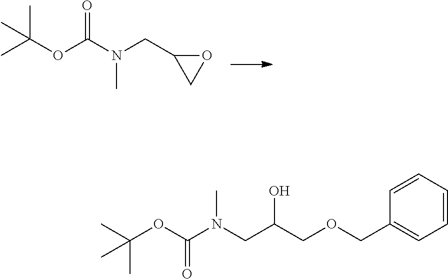 [SMILES: CN(CC1CO1)C(=O)OC(C)(C)C ; CN(CC(O)COCc1ccccc1)C(=O)OC(C)(C)C]

To a suspension of the tert-butyl N-methyl-N-(oxiran-2-ylmethyl)carbamate (2.0 g, 10.68 mmol) stirring in benzyl alcohol (30 mL, 289.9 mmol) at rt was added lithium hydroxide (6.5 g, 154.9 mmol) and the resulting solution was stirred overnight. The reaction mixture was diluted with DCM and water, separated and washed the aqueous with DCM once more. A solution of 1 M citric acid was required to separate the layers (adjusted mixture to pH ˜5). Then the organic layers were combined, dried over sodium sulfate, filtered and concentrated. The residue was then purified by silica gel chromatography (40 gram column) using a gradient from 100% dichloromethane to 20% methanol in dichloromethane to isolate a clear oil, tert-butyl N-(3-benzyloxy-2-hydroxy-propyl)-N-methyl-carbamate (2.8 g, 89%) ESI-MS m/z calc. 295.17834, found 296.3 (M+1)⁺; Retention time: 1.46 minutes; LC method A. ¹H NMR (400 MHz, DMSO-d₆) δ 7.33 (dd, J 10.2, 5.1 Hz, 4H), 7.28-7.20 (m, 1H), 5.17 (t, J 5.7 Hz, 1H), 4.96-4.84 (m, 1H), 4.49 (d, J 5.7 Hz, 3H), 3.81 (dt, J 7.2, 5.1 Hz, 1H), 3.17 (d, J 5.1 Hz, 1H), 3.15-2.87 (m, 1H), 2.86-2.73 (m, 3H), 1.44-1.30 (m, 9H).

### Step 3: 1-Benzyloxy-3-(methylamino)propan-2-ol

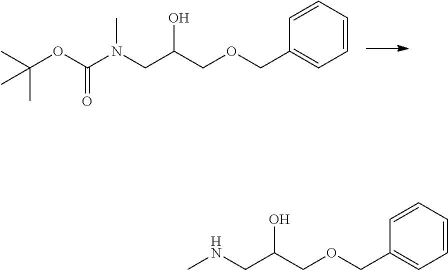 [SMILES: CN(CC(O)COCc1ccccc1)C(=O)OC(C)(C)C ; CNCC(O)COCc1ccccc1]

tert-Butyl N-(3-benzyloxy-2-hydroxy-propyl)-N-methyl-carbamate (2.8 g, 9.480 mmol) was dissolved in DCM (50 mL) and to the mixture was added HCl in dioxane (27 mL of 4 M, 108.0 mmol) and stirred at room temperature for 90 min. The reaction mixture was concentrated under reduced pressure to an oily orange residue, which was then slurried in diethyl ether (2×125 mL). The solid was collected by vacuum filtration to provide as an orange gum 1-benzyloxy-3-(methylamino)propan-2-ol (hydrochloride salt) (1.5 g, 68%) ESI-MS m z calc. 195.12593, found 196.2 (M+1)⁺; Retention time: 0.56 minutes; LC method A.

### Step 4: 3-[[4-[1-(Benzyloxymethyl)-2-[tert-butoxycarbonyl(methyl)amino]ethoxy]-6-(2,6-dimethylphenyl)pyrimidin-2-yl]sulfamoyl]benzoic acid

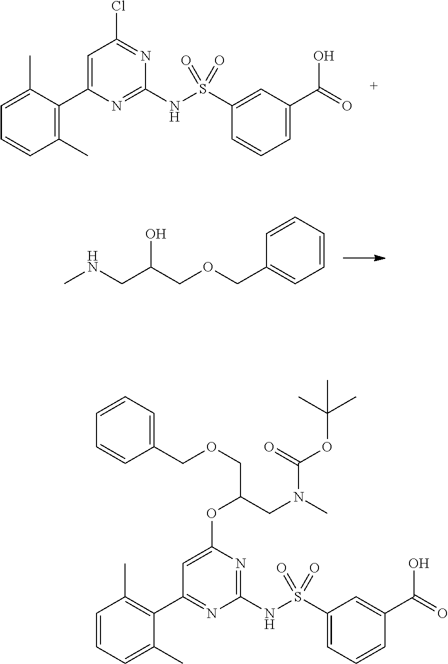 [SMILES: Cc1cccc(C)c1-c1cc(Cl)nc(NS(=O)(=O)c2cccc(C(=O)O)c2)n1 ; CNCC(O)COCc1ccccc1 ; Cc1cccc(C)c1-c1cc(OC(COCc2ccccc2)CN(C)C(=O)OC(C)(C)C)nc(NS(=O)(=O)c2cccc(C(=O)O)c2)n1]

In a 250 mL flask, 3-[[4-chloro-6-(2,6-dimethylphenyl)pyrimidin-2-yl]sulfamoyl]benzoic acid (approximately 2.808 g, 6.720 mmol), 1-benzyloxy-3-(methylamino)propan-2-ol (hydrochloride salt) (1.5 g, 6.473 mmol) and THE (75 mL) were mixed and cooled in an ice bath at 0° C., to which KOtBu (3.4 g, 30.30 mmol) was added. This mixture was stirred for 30 min at 0° C. and di-tert-butyl dicarbonate (2.2 g, 10.08 mmol) was added and allowed to stir for 5 hours. The mixture was then diluted with ethyl acetate and quenched with saturated ammonium chloride solution and then extracted with additional ethyl acetate (3×75 mL). The combined organic extract was washed with water (50 mL) and saturated brine solution (50 mL), then dried over sodium sulfate, filtered, and evaporated in vacuo. This crude product was purified on silica gel chromatography (40 gram column) using a gradient from 100% hexanes to 100% ethyl acetate to afford a white solid 3-[[4-[1-(benzyloxymethyl)-2-[tert-butoxycarbonyl(methyl)amino]ethoxy]-6-(2,6-dimethylphenyl)pyrimidin-2-yl]sulfamoyl]benzoic acid (1.14 g, 25%) ESI-MS m/z calc. 676.2567, found 677.2 (M+1)⁺; Retention time: 1.91 minutes, LC method A.

### Step 5: 3-[[4-[1-(Benzyloxymethyl)-2-(methylamino)ethoxy]-6-(2,6-dimethylphenyl)pyrimidin-2-yl]sulfamoyl]benzoic acid

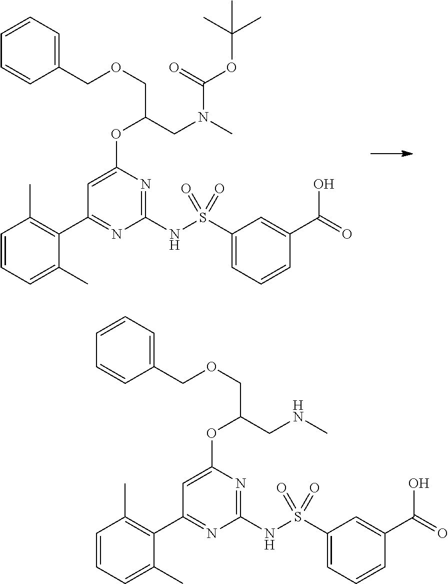 [SMILES: Cc1cccc(C)c1-c1cc(OC(COCc2ccccc2)CN(C)C(=O)OC(C)(C)C)nc(NS(=O)(=O)c2cccc(C(=O)O)c2)n1 ; CNCC(COCc1ccccc1)Oc1cc(-c2c(C)cccc2C)nc(NS(=O)(=O)c2cccc(C(=O)O)c2)n1]

3-[[4-[1-(Benzyloxymethyl)-2-[tert-butoxycarbonyl(methyl)amino]ethoxy]-6-(2,6-dimethylphenyl)pyrimidin-2-yl]sulfamoyl]benzoic acid (1.14 g, 1.684 mmol) was dissolved in DCM (30 mL) and to the mixture was added HCl in dioxane (4.75 mL of 4 M, 19.00 mmol) and stirred at room temperature for 90 min. The reaction mixture was concentrated under reduced pressure to a white solid, which was then slurried in diethyl ether (2×10 mL). The solid was collected by vacuum filtration to provide as a white solid 3-[[4-[1-(benzyloxymethyl)-2-(methylamino)ethoxy]-6-(2,6-dimethylphenyl)pyrimidin-2-yl]sulfamoyl]benzoic acid (hydrochloride salt)(1.03 g, 100%). ESI-MS m/z calc. 576.2043, found 577.3 (M+1)⁺; Retention time: 1.16 minutes (LC method D).

### Step 6: 10-(Benzyloxymethyl)-6-(2,6-dimethylphenyl)-12-methyl-2,2-dioxo-9-oxa-2)⁶-thia-3,5,12,19-tetrazatricyclo[12.3.1.14,8]nonadeca-1(18),4(19),5,7,14,16-hexaen-13-one (Compound 99)

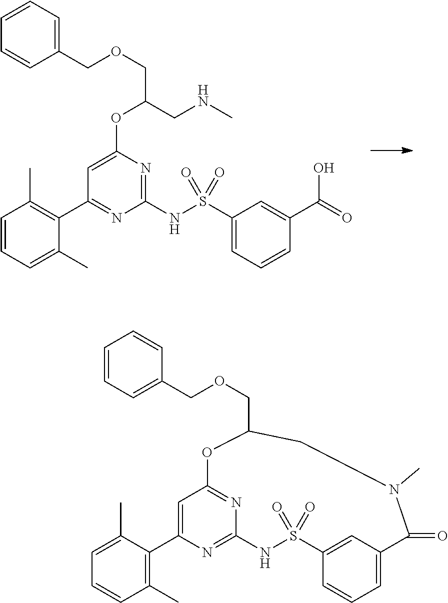 [SMILES: CNCC(COCc1ccccc1)Oc1cc(-c2c(C)cccc2C)nc(NS(=O)(=O)c2cccc(C(=O)O)c2)n1 ; Cc1cccc(C)c1-c1cc2nc(n1)NS(=O)(=O)c1cccc(c1)C(=O)N(C)CC(COCc1ccccc1)O2]

In a 100-mL flask, 3-[[4-[1-(benzyloxymethyl)-2-(methylamino)ethoxy]-6-(2,6-dimethylphenyl)pyrimidin-2-yl]sulfamoyl]benzoic acid (hydrochloride salt) (1.03 g, 1.680 mmol) was dissolved in DMF (50 mL), to which DIPEA (2.1 mL, 12.06 mmol) and HATU (960 mg, 2.525 mmol) were added. After stirring at room temperature for 15 min, the mixture was diluted with aqueous saturated ammonium chloride solution and extracted with ethyl acetate (3×25 mL). The combined organic extract were washed with a saturated brine solution (50 mL), then dried over sodium sulfate, filtered, and evaporated in vacuo. This crude product was purified on silica gel chromatography (40 gram column) using a gradient from 100% hexanes to 100% ethyl acetate followed by a second silica gel chromatography (24 gram column) using a gradient from 100% dichloromethane to 10% methanol in dichloromethane to afford a white solid 10-(benzyloxymethyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (545 mg, 58%) ESI-MS m/z calc. 558.19366, found 559.2 (M+1)⁺; Retention time: 1.64 minutes. LC method A. ¹H NMR (400 MHz, DMSO-d₆) δ 8.46 (s, 1H), 7.89 (s, 1H), 7.63 (s, 2H), 7.41-7.35 (m, 4H), 7.33-7.28 (m, 1H), 7.25 (t, J 7.5 Hz, 1H), 7.12 (d, J 7.6 Hz, 2H), 6.31 (s, 1H), 5.69 (s, 1H), 4.63 (q, J=11.8 Hz, 2H), 4.04-3.94 (m, 2H), 3.31 (d, J=7.7 Hz, 3H), 3.05 (s, 3H), 2.05 (d, J=21.1 Hz, 6H).

### Example 77: Preparation of Compound 100

### Step 1: 6-(2,6-Dimethylphenyl)-10-(hydroxymethyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one

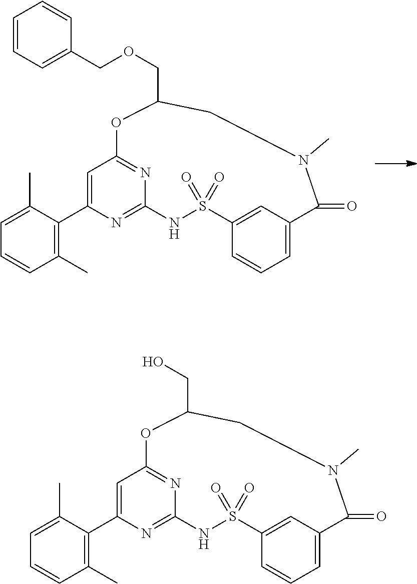 [SMILES: Cc1cccc(C)c1-c1cc2nc(n1)NS(=O)(=O)c1cccc(c1)C(=O)N(C)CC(COCc1ccccc1)O2 ; Cc1cccc(C)c1-c1cc2nc(n1)NS(=O)(=O)c1cccc(c1)C(=O)N(C)CC(CO)O2]

A mixture of 10-(benzyloxymethyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (545 mg, 0.9756 mmol) in MeOH (15 mL) was purged with nitrogen followed by Pd/C (105 mg of 10% w/w, 0.09867 mmol) and stirred under a hydrogen atmosphere for 18 h. The reaction mixture was purged with nitrogen, filtered over Celite, and washed with excess methanol and then concentrated. The material was dried to afford a white solid 6-(2,6-dimethylphenyl)-10-(hydroxymethyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (340 mg, 74%) ESI-MS m/z calc. 468.14673, found 469.2 (M+1)⁺; Retention time: 0.98 minutes; LC method A.

### Step 2: 6-(2,6-Dimethylphenyl)-12-methyl-2,2,13-trioxo-9-oxa-2λ⁶-thia-3,5,12,19-tetrazatricyclo[12.3.1.14,8]nonadeca-1(18),4(19),5,7,14,16-hexaene-10-carboxylic acid

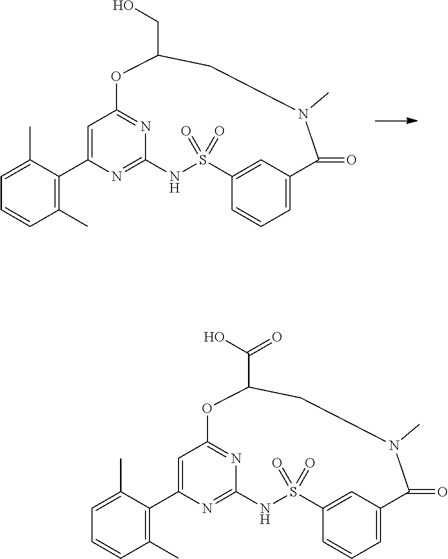 [SMILES: Cc1cccc(C)c1-c1cc2nc(n1)NS(=O)(=O)c1cccc(c1)C(=O)N(C)CC(CO)O2 ; Cc1cccc(C)c1-c1cc2nc(n1)NS(=O)(=O)c1cccc(c1)C(=O)N(C)CC(C(=O)O)O2]

Dess-Martin periodinane (375 mg, 0.8841 mmol) was added to a stirred solution of 6-(2,6-dimethylphenyl)-10-(hydroxymethyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (340 mg, 0.7257 mmol) in anhydrous DCM (8.0 mL) at 0° C. (ice-water bath) under nitrogen. After 15 min, the bath was removed, and the reaction was allowed to warm to ambient temperature and stirring continued for another 1 h. The reaction was diluted with DCM (40 mL) and saturated aqueous sodium bicarbonate (30 mL) was added slowly. Then a solution of 10% sodium thiosulfate (10 mL) was added and stirred at ambient temperature for 30 min (hazy solution). The layers were separated and the aqueous layer was extracted with DCM:MeOH 9:1 (3×30 mL). The combined organics were washed with brine (30 mL), dried over anhydrous sodium sulfate, filtered and concentrated under reduced pressure. This crude product was purified on silica gel chromatography (24 gram column) using a gradient from 100% hexanes to 100% ethyl acetate and to afford an off-white solid 6-(2,6-dimethylphenyl)-12-methyl-2,2,13-trioxo-9-oxa-2λ⁶-thia-3,5,12,19-tetrazatricyclo[12.3.1.14,8]nonadeca-1(18),4(19),5,7,14,16-hexaene-10-carbaldehyde (150 mg, 44%) ESI-MS m/z calc. 466.1311, found 467.2 (M+1)⁺; Retention time: 0.88 minutes; LC method A.

### Step 3: 10-[(4-tert-Butylpiperazin-1-yl)methyl]-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 100)

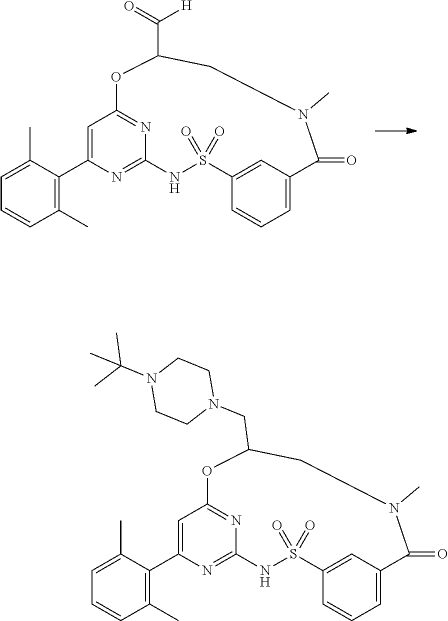 [SMILES: Cc1cccc(C)c1-c1cc2nc(n1)NS(=O)(=O)c1cccc(c1)C(=O)N(C)CC(C=O)O2 ; Cc1cccc(C)c1-c1cc2nc(n1)NS(=O)(=O)c1cccc(c1)C(=O)N(C)CC(CN1CCN(C(C)(C)C)CC1)O2]

In a 20 mL scintillation vial, 6-(2,6-dimethylphenyl)-12-methyl-2,2,13-trioxo-9-oxa-2λ⁶-thia-3,5,12,19-tetrazatricyclo[12.3.1.14,8]nonadeca-1(18),4(19),5,7,14,16-hexaene-10-carbaldehyde (30 mg, 0.06431 mmol) was dissolved in DMF (1.0 mL). Then 1-tert-butylpiperazine (23 mg, 0.1617 mmol) was added and stirred for 15 minutes. Then sodium triacetoxyborohydride (35 mg, 0.1651 mmol) was added portionwise and the solution was stirred for 1 hour at RT. The mixture was quenched with minimal water (drops) then the mixture was filtered and was purified by reverse-phase preparative chromatography utilizing a Cis column and a gradient of 1-30% acetonitrile-water+5 mM HCl to afford a white solid 10-[(4-tert-butylpiperazin-1-yl)methyl]-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (dihydrochloride salt) (3.3 mg, 8%) ESI-MS m/z calc. 592.2832, found 593.4 (M+1)⁺; Retention time: 0.92 minutes; LC method A. ¹H NMR (400 MHz, DMSO-d₆) δ 8.54 (s, 1H), 7.89 (d, J 7.0 Hz, 1H), 7.64 (d, J 7.6 Hz, 2H), 7.26 (t, J 7.7 Hz, 1H), 7.13 (d, J 7.7 Hz, 2H), 6.33 (s, 1H), 5.90 (s, 1H), 3.75-3.69 (m, 8H), 3.50-3.40 (m, 3H), 3.24 (dd, J 14.6, 10.5 Hz, 2H), 3.11-3.07 (m, 3H), 2.07 (s, 6H), 1.37 (s, 9H).

### Example 78: Preparation of Compound 101

### Step 1: tert-Butyl N-[2-(5-tert-butyl-2-pyridyl)-2-oxo-ethyl]-N-methyl-carbamate

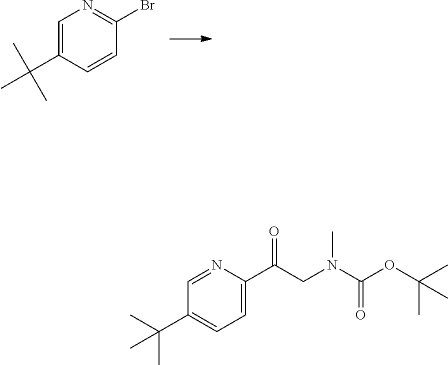 [SMILES: CC(C)(C)c1ccc(Br)nc1 ; CN(CC(=O)c1ccc(C(C)(C)C)cn1)C(=O)OC(C)(C)C]

To a solution of 2-bromo-5-tert-butyl-pyridine (1 g, 4.671 mmol) in THE (15 mL) at −78° C. was added n-BuLi (2.5 M in hexanes (2.4 mL of 2.5 M, 6.000 mmol) dropwise and the reaction mixture was stirred at this temperature for 15 min before the addition of tert-butyl N-[2-[methoxy(methyl)amino]-2-oxo-ethyl]-N-methyl-carbamate (1.3 g, 5.597 mmol). The cooling bath was removed, and the reaction mixture stirred for 2 hours. The reaction mixture was quenched with ammonium chloride, poured into water, the pH brought to 8 with saturated aqueous sodium bicarbonate, then extracted with EtOAc (3×). The organics were combined, washed with brine, dried over sodium sulfate and evaporated to dryness. Purification by column chromatography (80 g silica; 0-50% EtOAc in hexanes) gave tert-butyl N-[2-(5-tert-butyl-2-pyridyl)-2-oxo-ethyl]-N-methyl-carbamate (710 mg, 50%) as a clear oil. ESI-MS m/z calc. 306.19434, found 307.2 (M+1)⁺; Retention time: 0.71 minutes; LC method D.

### Step 2: 1-(5-tert-Butyl-2-pyridyl)-2-(methylamino)ethanol

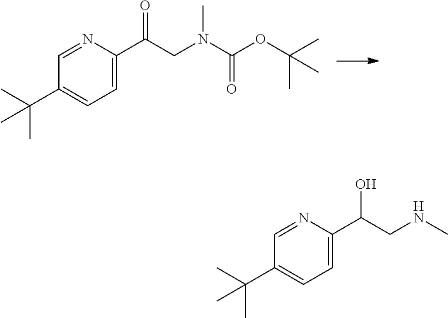

To a solution of tert-butyl N-[2-(5-tert-butyl-2-pyridyl)-2-oxo-ethyl]-N-methyl-carbamate (710 mg, 2.317 mmol) in MeOH (10 mL) was added sodium borohydride (52 mg, 1.374 mmol) and the reaction mixture stirred at room temperature for 1 hour. The reaction mixture was poured into water and extracted with EtOAc (3×). The organic layers were combined, washed with water then brine, dried over sodium sulfate and evaporated to dryness. The residue was taken up in DCM (10 mL), treated with HCl in dioxane (12 mL of 4 M, 48.00 mmol) and the reaction mixture stirred at room temperature for 20 min. Evaporation gave 1-(5-tert-butyl-2-pyridyl)-2-(methylamino)ethanol (hydrochloride salt) (530 mg, 93%).ESI-MS m/z calc. 208.15756, found 209.2 (M+1)⁺; Retention time: 0.24 minutes; (LC method D).

### Step 3: 3-[[4-[2-[tert-Butoxycarbonyl(methyl)amino]-1-(5-tert-butyl-2-pyridyl)ethoxy]-6-(2,6-dimethylphenyl)pyrimidin-2-yl]sulfamoyl]benzoic acid

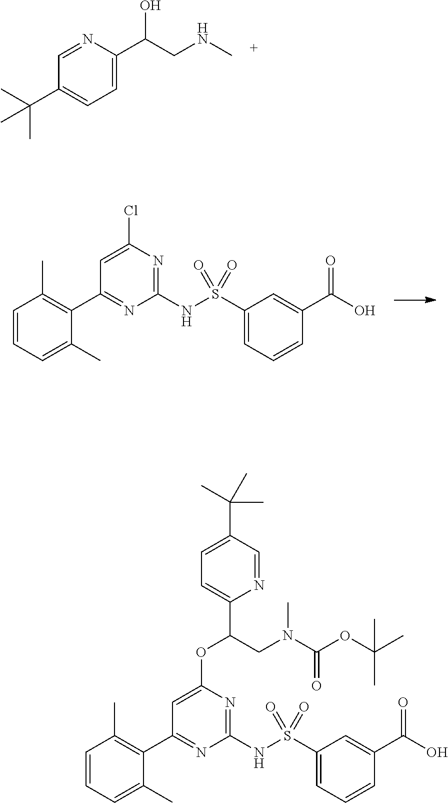 [SMILES: CNCC(O)c1ccc(C(C)(C)C)cn1 ; Cc1cccc(C)c1-c1cc(Cl)nc(NS(=O)(=O)c2cccc(C(=O)O)c2)n1 ; Cc1cccc(C)c1-c1cc(OC(CN(C)C(=O)OC(C)(C)C)c2ccc(C(C)(C)C)cn2)nc(NS(=O)(=O)c2cccc(C(=O)O)c2)n1]

In a 50 mL flask, 1-(5-tert-butyl-2-pyridyl)-2-(methylamino)ethanol (hydrochloride salt) (327.8 mg, 1.339 mmol), 3-[[4-chloro-6-(2,6-dimethylphenyl)pyrimidin-2-yl]sulfamoyl]benzoic acid (671.3 mg, 1.606 mmol), and THE (15 mL) was cooled to 0° C. and treated with NaOtBu (910.2 mg, 9.471 mmol). The reaction was warmed to room temperature and stirred for 5.5 h. To the reaction solution at room temperature was added tert-butoxycarbonyl tert-butyl carbonate (569.1 mg, 2.608 mmol). The reaction solution was allowed to stir at room temperature overnight. The reaction mixture was diluted with EtOAc and quenched with aqueous saturated ammonium chloride. The organics phase was separated, washed with brine, dried over sodium sulfate and evaporated to give 3-[[4-[2-[tert-butoxycarbonyl(methyl)amino]-1-(5-tert-butyl-2-pyridyl)ethoxy]-6-(2,6-dimethylphenyl)pyrimidin-2-yl]sulfamoyl]benzoic acid (253.7 mg, 27%) ESI-MS m/z calc. 689.2883, found 690.4 (M+1)⁺; Retention time: 0.67 minutes; (LC method D).

### Step 4: 10-(5-tert-Butyl-2-pyridyl)-6-(2,6-dimethylphenyl)-12-methyl-2,2-dioxo-9-oxa-2)⁶-thia-3,5,12,19-tetrazatricyclo[12.3.1.14,8]nonadeca-1(18),4(19),5,7,14,16-hexaen-13-one (Compound 101)

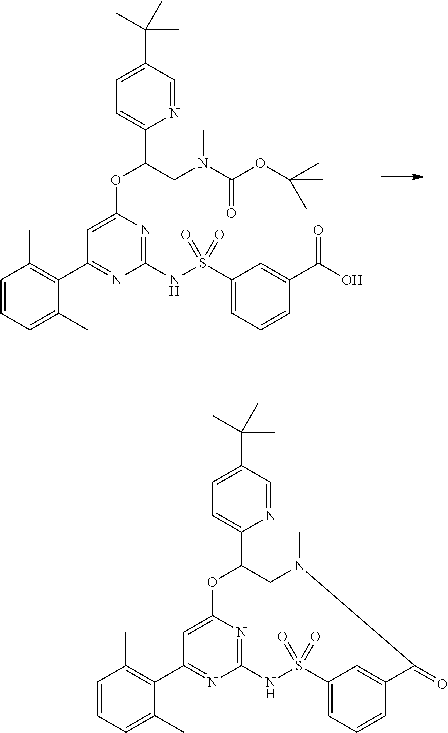 [SMILES: Cc1cccc(C)c1-c1cc(OC(CN(C)C(=O)OC(C)(C)C)c2ccc(C(C)(C)C)cn2)nc(NS(=O)(=O)c2cccc(C(=O)O)c2)n1 ; Cc1cccc(C)c1-c1cc2nc(n1)NS(=O)(=O)c1cccc(c1)C(=O)N(C)CC(c1ccc(C(C)(C)C)cn1)O2]

A dioxane (1 mL) solution of 3-[[4-[2-[tert-butoxycarbonyl(methyl)amino]-1-(5-tert-butyl-2-pyridyl)ethoxy]-6-(2,6-dimethylphenyl)pyrimidin-2-yl]sulfamoyl]benzoic acid (253.7 mg, 0.3678 mmol) and HCl (3000 μL of 4 M, 12.00 mmol) was stirred at room temperature for 1 h and then concentrated in vacuo. The crude residue was taken up in DMF (35 mL) and treated with HATU (210.2 mg, 0.5528 mmol) followed by DIPEA (450 μL, 2.584 mmol). The reaction mixture was stirred for 5 minutes and then the DMF was evaporated in vacuo. The solutions were filtered and the filtrate was purified by reverse phase using a 15 min gradient of 1-99% MeCN in water (HCl modifier) to give 10-(5-tert-butyl-2-pyridyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (123.9 mg, 55%). ESI-MS m/z calc. 571.22534, found 572.33 (M+1)⁺; Retention time: 1.6 minutes; LC method A. ¹H NMR (400 MHz, DMSO-d₆) δ 8.79 (dd, J 2.4, 0.8 Hz, 1H), 8.63 (s, 1H), 8.01 (dd, J 8.2, 2.5 Hz, 1H), 7.94 (d, J 7.3 Hz, 1H), 7.76 (d, J 8.2 Hz, 1H), 7.73-7.58 (m, 2H), 7.30-7.20 (m, 1H), 7.11 (d, J 7.7 Hz, 2H), 6.45 (dd, J 10.8, 4.3 Hz, 1H), 6.31 (s, 1H), 3.75 (dd, J 14.3, 4.2 Hz, 1H), 3.63 (dd, J=14.2, 10.7 Hz, 1H), 2.21 (s, 3H), 2.04 (s, 6H), 1.36 (s, 9H).

### Example 79: Preparation of Compound 102 and Compound 103

### Step 1: 10-(5-tert-Butyl-2-pyridyl)-6-(2,6-dimethylphenyl)-12-methyl-2,2-dioxo-9-oxa-2)⁶-thia-3,5,12,19-tetrazatricyclo[12.3.1.14,8]nonadeca-1(18),4(19),5,7,14,16-hexaen-13-one, enantiomer 1 (Compound 102), and 10-(5-tert-butyl-2-pyridyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one, enantiomer 2 (Compound 103)

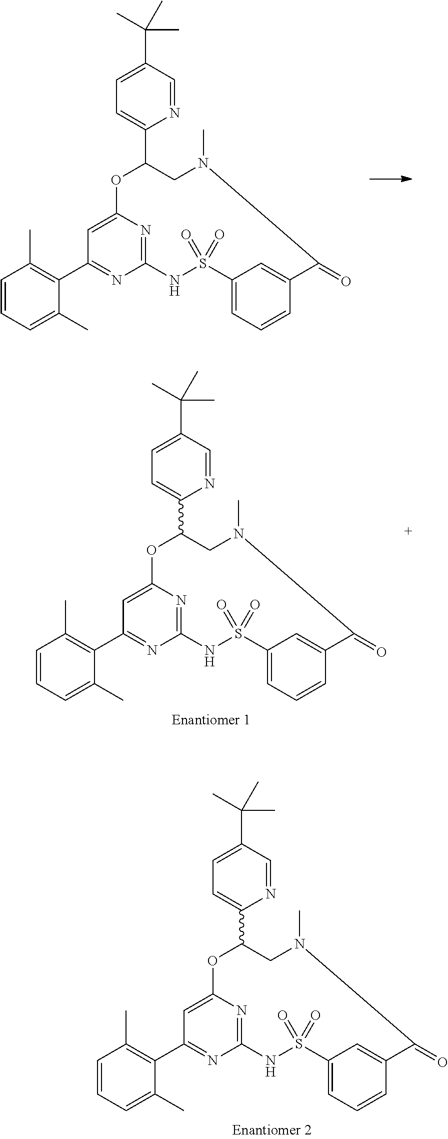 [SMILES: Cc1cccc(C)c1-c1cc2nc(n1)NS(=O)(=O)c1cccc(c1)C(=O)N(C)CC(c1ccc(C(C)(C)C)cn1)O2 ; Cc1cccc(C)c1-c1cc2nc(n1)NS(=O)(=O)c1cccc(c1)C(=O)N(C)CC(c1ccc(C(C)(C)C)cn1)O2 ; Cc1cccc(C)c1-c1cc2nc(n1)NS(=O)(=O)c1cccc(c1)C(=O)N(C)CC(c1ccc(C(C)(C)C)cn1)O2]

Racemic 10-(5-tert-butyl-2-pyridyl)-6-(2,6-dimethylphenyl)-12-methyl-2,2-dioxo-9-oxa-2λ⁶-thia-3, 5,12,19-tetrazatricyclo[12.3.1.14,8]nonadeca-1(18),4(19),5,7,14,16-hexaen-13-one (hydrochloride salt) (100 mg, 0.1644 mmol) was subjected to chiral SFC using a (R,R)-Whelk-O (250×21 mm, 5 m) column at 40° C., a mobile phase comprised of 80% MeOH (ammonia modifier) and 20% C02, with a flow of 40 mL/min, at a concentration of 50 mg/mL in MeOH (20 mM NH₃), and with an injection volume of 300 μL, and utilizing a 220/254 nm wavelength to give two enantiomers: Enantiomer 1, peak 1, 10-(5-tert-butyl-2-pyridyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (37.1 mg, 37%)¹H NMR (400 MHz, DMSO-d₆) δ 8.80 (dd, J=2.4, 0.8 Hz, 1H), 8.64 (s, 1H), 8.04 (dd, J 8.2, 2.5 Hz, 1H), 7.94 (d, J 7.3 Hz, 1H), 7.78 (d, J 8.2 Hz, 1H), 7.67 (q, J 7.9 Hz, 2H), 7.25 (t, J 7.6 Hz, 1H), 7.11 (d, J 7.6 Hz, 2H), 6.47 (dd, J 10.7, 4.3 Hz, 1H), 6.32 (s, 1H), 3.76 (dd, J=14.2, 4.2 Hz, 1H), 3.63 (dd, J=14.2, 10.7 Hz, 1H), 2.21 (s, 3H), 2.04 (s, 6H), 1.37 (s, 9H). ESI-MS m/z calc. 571.22534, found 572.5 (M+1)⁺; Retention time: 1.6 minutes, (LC method A); and enantiomer 2, peak 2, 10-(5-tert-butyl-2-pyridyl)-6-(2,6-dimethylphenyl)-12-methyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (35.2 mg, 35%)¹H NMR (400 MHz, DMSO-d₆) δ 8.80 (d, J 2.4 Hz, 1H), 8.64 (s, 1H), 8.04 (d, J 8.0 Hz, 1H), 7.94 (d, J 7.3 Hz, 1H), 7.79 (d, J 8.2 Hz, 1H), 7.74-7.60 (m, 2H), 7.25 (t, J 7.6 Hz, 1H), 7.11 (d, J 7.6 Hz, 2H), 6.47 (dd, J 10.8, 4.2 Hz, 1H), 6.32 (s, 1H), 3.76 (dd, J=14.4, 4.3 Hz, 1H), 3.63 (dd, J=14.3, 10.8 Hz, 1H), 2.21 (s, 3H), 2.04 (s, 6H), 1.37 (s, 9H). ESI-MS m/z calc. 571.22534, found 572.4 (M+1)⁺; Retention time: 1.62 minutes; (LC method A).

### Example 80: Preparation of Compound 104 and Compound 105

### Step 1: N-Methoxy-N-methyl-1-(trifluoromethyl)cyclopropanecarboxamide

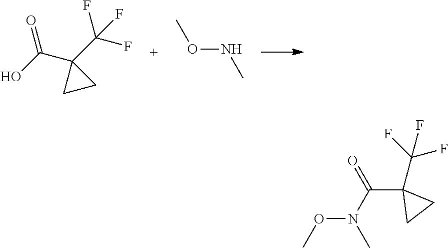

1,1′-Carbonyldiimidazole (14.0 g, 86.340 mmol) was added portionwise to a solution of 1-(trifluoromethyl)cyclopropanecarboxylic acid (10 g, 64.898 mmol) in dichloromethane (100 mL) at 15° C. in 15 min. The mixture was stirred at 20° C. for 2 h. Triethylamine (9.2 g, 12.672 mL, 90.918 mmol), then N-methoxymethanamine (hydrochloride salt) (8.9 g, 91.241 mmol) were added and the mixture was stirred at room temperature 18 h. A hydrochloric acid solution (3 N, 65 mL) was added at 5° C. and phases were separated. The organic layer was washed with a potassium bicarbonate solution (10% w/w, 20 mL), dried over sodium sulfate and concentrated under reduced pressure to afford N-methoxy-N-methyl-1-(trifluoromethyl)cyclopropanecarboxamide (11.87 g, 93%). ¹H NMR (400 MHz, CDCl₃) δ 3.74 (s, 3H), 3.28 (s, 3H), 1.36-1.17 (m, 4H).

### Step 2: (6-Bromo-3-pyridyl)-[1-(trifluoromethyl)cyclopropyl]methanone

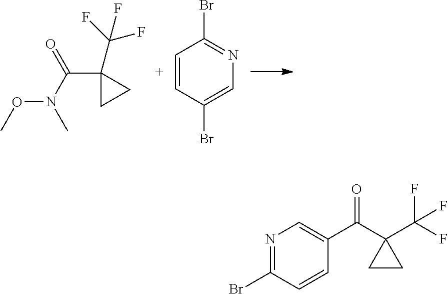

2,5-Dibromopyridine (57 g, 240.62 mmol) was dissolved in diethyl ether (1000 mL) then cooled to −78° C. Then n-BuLi in hexanes (120 mL of 2.5 M, 300.00 mmol) was added drop wise via addition funnel keeping the internal reaction temperature below −70° C. The mixture was stirred for an hour, then N-methoxy-N-methyl-1-(trifluoromethyl)cyclopropanecarboxamide (59.45 g, 301.54 mmol) in diethyl ether (150 mL) was added to the mixture via addition funnel and the reaction was stirred at −78° C. for 1 hour. The reaction was allowed to warm to −10° C., then quenched with NH₄Cl solution (500 mL). The phases were separated, and the aqueous layer was extracted with diethyl ether (200 mL). The organic layer was washed with water (200 mL) and brine (200 mL) and then dried over sodium sulfate and concentrated. The crude residue was combined with another product batch from another reaction run on a similar scale and loaded on to silica gel and purified by flash column chromatography (220 g+330 g column) using 0-20% EtOAc in hexanes. The appropriate fractions were collected to give (6-bromo-3-pyridyl)-[1-(trifluoromethyl)cyclopropyl]methanone (86.41 g, 85% corrected yield). ESI-MS m/z calc. 292.9663, found 294.2 (M+1)⁺; Retention time: 3.17 minutes; LC method T.

### Step 3: (6-Bromo-3-pyridyl)-[1-(trifluoromethyl)cyclopropyl]methanol

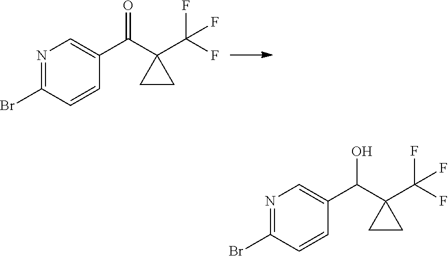 [SMILES: O=C(c1ccc(Br)nc1)C1(C(F)(F)F)CC1 ; OC(c1ccc(Br)nc1)C1(C(F)(F)F)CC1]

(6-Bromo-3-pyridyl)-[1-(trifluoromethyl)cyclopropyl]methanone (86.41 g, 293.84 mmol) was dissolved in EtOH (1000 mL) then sodium borohydride (12.46 g, 329.35 mmol) was added portion-wise at room temperature. The reaction was allowed to stir for 1.5 hours, then quenched with acetone (750 mL). The volatiles were removed under vacuum and the crude residue was taken up in EtOAc (700 mL) and washed with water and brine (400 mL each). The organic layer was dried over sodium sulfate and concentrated. The crude residue was loaded on to silica gel and purified by flash column chromatography using 0-30% EtOAc in hexanes to give (6-bromo-3-pyridyl)-[1-(trifluoromethyl)cyclopropyl]methanol (74.38 g, 51%) as a colorless oil. ESI-MS m/z calc. 294.98196, found 296.2 (M+1)⁺; Retention time: 2.7 minutes; LC method T.

### Step 4: [(6-Bromo-3-pyridyl)-[1-(trifluoromethyl)cyclopropyl]methyl]methanesulfonate

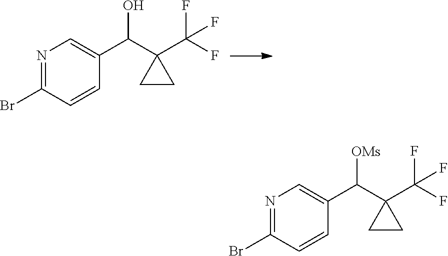 [SMILES: OC(c1ccc(Br)nc1)C1(C(F)(F)F)CC1 ; CS(=O)(=O)OC(c1ccc(Br)nc1)C1(C(F)(F)F)CC1]

(6-Bromo-3-pyridyl)-[1-(trifluoromethyl)cyclopropyl]methanol (74.38 g, 200.97 mmol) was dissolved in pyridine (225 mL) then cooled to 0° C. MsCl (28.860 g, 19.5 mL, 251.94 mmol) was added via syringe and the ice bath was removed and the reaction stirred at room temperature. After 4.5 hours the reaction was quenched by the addition of 1 L EtOAc, then 600 mL water. The layers were separated, and the organic layer washed with 1M HCl (600 mL), sat. sodium bicarbonate solution (600 mL) and brine (4×150 mL). The organic layer was dried over sodium sulfate and concentrated. The crude residue was dry loaded on to silica gel and purified by flash column chromatography using 0-30% EtOAc in hexanes. The crude material was loaded on to silica gel again and purified using 1-5% acetone in DCM to give good separation and the appropriate fractions were collected to give [(6-bromo-3-pyridyl)-[1-(trifluoromethyl)cyclopropyl]methyl] methanesulfonate (35.34 g, 45%) as colorless crystals. ¹H NMR (500 MHz, DMSO-d₆) δ 8.52 (d, J 2.5 Hz, 1H), 7.86 (dd, J 8.3, 2.6 Hz, 1H), 7.74 (d, J=8.3 Hz, 1H), 5.75 (s, 1H), 3.23 (s, 3H), 1.55-1.47 (m, 1H), 1.26 (dt, J 9.9, 6.1 Hz, 1H), 1.17 (ddd, J 9.9, 7.0, 5.5 Hz, 1H), 1.01 (qt, J 6.9, 5.2, 2.3 Hz, 1H). ESI-MS m/z calc. 372.9595, found 374.1 (M+1)⁺; Retention time: 3.04 minutes; LC method T.

### Step 5: 2-Bromo-5-[[1-(trifluoromethyl)cyclopropyl]methyl]pyridine

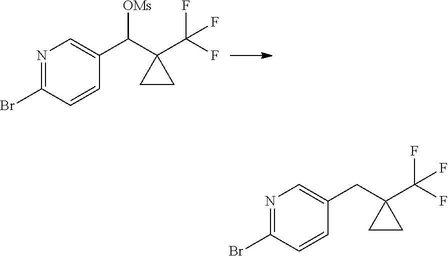 [SMILES: CS(=O)(=O)OC(c1ccc(Br)nc1)C1(C(F)(F)F)CC1 ; FC(F)(F)C1(Cc2ccc(Br)nc2)CC1]

The reaction was run in 7 separate batches using each 1.5 g of [(6-bromo-3-pyridyl)-[1-(trifluoromethyl)cyclopropyl]methyl] methanesulfonate and 1 batch using 1.17 g of [(6-bromo-3-pyridyl)-[1-(trifluoromethyl)cyclopropyl]methyl] methanesulfonate. DMF (20 mL) was added to a heat-dried 100 mL flask under nitrogen, and purged with nitrogen gas for 20 min. [(6-Bromo-3-pyridyl)-[1-(trifluoromethyl)cyclopropyl]methyl] methanesulfonate (1.5 g) was added followed by sodium borohydride (520 mg) and the mixture was placed in a pre-heated oil bath at 100° C. for 20 minutes. The mixture was cooled rapidly in a water bath, then diluted with water (100 mL) and combined with the other batches of the quenched material. The aqueous layer was extracted three times with EtOAc (200 mL each). The organic layers were washed with water (200 mL) and brine (3×150 mL), dried over sodium sulfate and concentrated. The crude residue was dry loaded on to silica gel and purified using flash column chromatography using 0-30% EtOAc in hexanes to give 2-bromo-5-[[1-(trifluoromethyl)cyclopropyl]methyl]pyridine (2.6103 g, 28%) as a colorless oil. ¹H NMR (250 MHz, DMSO-d₆) δ 8.32 (d, J 2.5 Hz, 1H), 7.71 (dd, J 8.2, 2.5 Hz, 1H), 7.61 (d, J 8.2 Hz, 1H), 2.94 (s, 2H), 1.06-0.85 (m, 4H).

### Step 6: tert-Butyl N-[2-[methoxy(methyl)amino]-2-oxo-ethyl]-N-methyl-carbamate

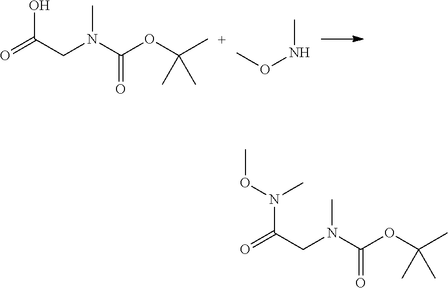

In a 1-L round-bottomed flask, 2-[tert-butoxycarbonyl(methyl)amino]acetic acid (25.85 g, 136.6 mmol), DCM (200 mL), DMF (200 mL), N-methoxymethanamine (hydrochloride salt)(17.10 g, 175.3 mmol), DIPEA (120 mL, 688.9 mmol), HOBt (24.22 g, 179.2 mmol) and EDCI (hydrochloride salt) (40 g, 175.3 mmol) were added in this order. This solution was stirred at room temperature for 3.5 h, after which it was diluted with ethyl acetate (1 L). This mixture was washed with 1N HCl solution (2×500 mL), 1N NaOH solution (2×500 mL), water (500 mL) and saturated aqueous sodium chloride solution (500 mL), then dried over sodium sulfate, filtered, and evaporated in vacuo to give a colorless liquid, tert-butyl N-[2-[methoxy(methyl)amino]-2-oxo-ethyl]-N-methyl-carbamate (25.1688 g, 79%)¹H NMR (400 MHz, dimethylsulfoxide-d6, mixture of 2 distinct rotamers) 6 4.08 and 4.07 (two singlets, 2H), 3.68 and 3.66 (two singlets, 3H), 3.10 and 3.09 (two singlets, 3H), 2.82 and 2.78 (two singlets, 3H), 1.39 and 1.33 (two singlets, 9H). ESI-MS m/z calc. 232.1423, found 233.0 (M+1)⁺; Retention time: 1.02 minutes; LC method A.

### Step 7: tert-Butyl N-methyl-N-[2-oxo-2-[5-[[1-(trifluoromethyl)cyclopropyl]methyl]-2-pyridyl]ethyl]carbamate

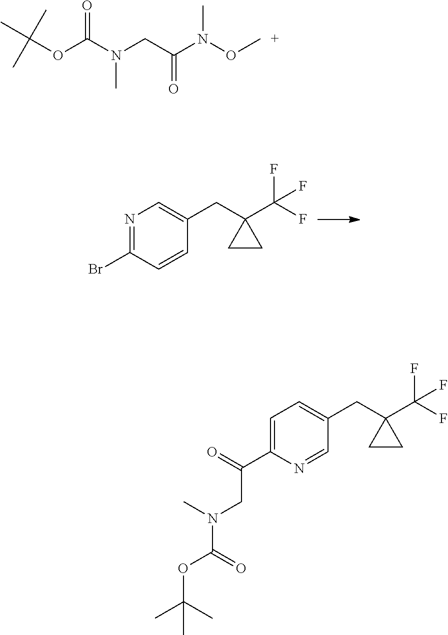

n-BuLi (12.6 mL of 2.5 M, 31.500 mmol) was added dropwise to a suspension of 2-bromo-5-[[1-(trifluoromethyl)cyclopropyl]methyl]pyridine (8 g, 28.563 mmol) in diethyl ether (110 mL) cooled at −78° C. The reaction was stirred for one hour at the same temperature, during which time it slowly turned to a red solution. A solution of tert-butyl N-[2-[methoxy(methyl)amino]-2-oxo-ethyl]-N-methyl-carbamate (9.2 g, 39.608 mmol) in diethyl ether (24 mL) was added dropwise. The obtained red solution was stirred at −78° C. for one hour, and then allowed to slowly warm up to −10° C. over one hour. The reaction was quenched with water (100 mL) and warmed up to rt. EtOAc (200 mL) was added. The two layers were separated, and the aqueous layer was extracted with EtOAc (200 mL). The combined organic layers were washed with brine (40 mL), dried over sodium sulfate and concentrated to provide crude product. The resulting crude was purified by companion (220 g, eluting 0 to 30% ethyl acetate/heptanes) to give tert-butyl N-methyl-N-[2-oxo-2-[5-[[1-(trifluoromethyl)cyclopropyl]methyl]-2-pyridyl]ethyl]carbamate (5.75 g, 54%) as yellow solid. ESI-MS m/z calc. 372.16608, found 373.2 (M+1)⁺; Retention time: 2.15 minutes; LC method K.

### Step 8: tert-Butyl N-[2-hydroxy-2-[5-[[1-(trifluoromethyl)cyclopropyl]methyl]-2-pyridyl]ethyl]-N-methyl-carbamate

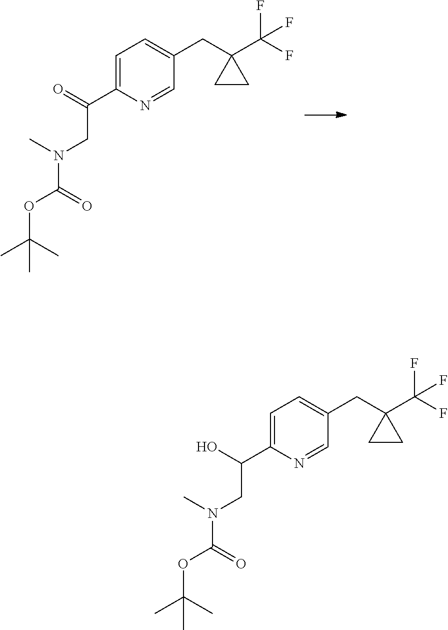 [SMILES: CN(CC(=O)c1ccc(CC2(C(F)(F)F)CC2)cn1)C(=O)OC(C)(C)C ; CN(CC(O)c1ccc(CC2(C(F)(F)F)CC2)cn1)C(=O)OC(C)(C)C]

Sodium borohydride (2.69 g, 71.103 mmol) was added to a solution of tert-butyl N-methyl-N-[2-oxo-2-[5-[[1-(trifluoromethyl)cyclopropyl]methyl]-2-pyridyl]ethyl]carbamate (14.3 g, 38.401 mmol) in MeOH (88 mL) and THE (22 mL) cooled with ice-water. The reaction was stirred at 0° C. for 20 minutes. The reaction was quenched with water (100 mL). The mixture was concentrated at 30° C. to remove most of organic solvents. The residue was extracted with EtOAc (200 mL×2). The combined organic extracts were washed with brine (40 mL), dried over sodium sulfate, filtered and concentrated under reduced pressure. The crude product was purified by flash chromatography (220 g silica gel, eluting 10 to 60% EtOAc/heptanes) to afford tert-butyl N-[2-hydroxy-2-[5-[[1-(trifluoromethyl)cyclopropyl]methyl]-2-pyridyl]ethyl]-N-methyl-carbamate (13.7 g, 95%) as light yellow oil. ESI-MS m/z calc. 374.18173, found 375.2 (M+1)⁺; Retention time: 1.81 minutes; LC method K.

### Step 9: 2-(Methylamino)-1-[5-[[1-(trifluoromethyl)cyclopropyl]methyl]-2-pyridyl]ethanol

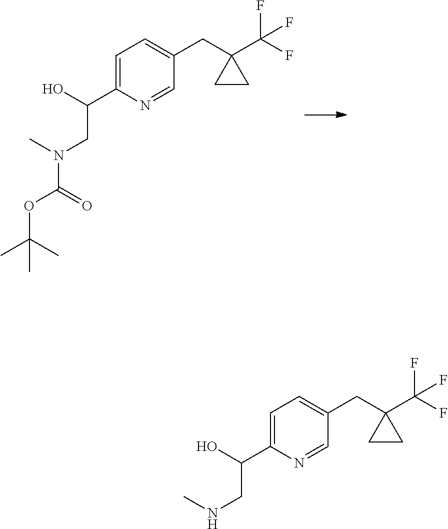 [SMILES: CN(CC(O)c1ccc(CC2(C(F)(F)F)CC2)cn1)C(=O)OC(C)(C)C ; CNCC(O)c1ccc(CC2(C(F)(F)F)CC2)cn1]

HCl in 1,4-dioxane (137 mL of 4 M, 548.00 mmol) was added to a solution of tert-butyl N-[2-hydroxy-2-[5-[[1-(trifluoromethyl)cyclopropyl]methyl]-2-pyridyl]ethyl]-N-methyl-carbamate (13.7 g, 36.592 mmol) in 1,4-dioxane (300 mL) at rt. After the addition, the mixture was stirred at rt overnight. A white suspension was obtained. The reaction was concentrated to dryness. The solid was triturated in a mixture of DCM (100 mL) and heptanes (200 mL). The obtained solid was filtered off and dried under high vacuum to afford 2-(methylamino)-1-[5-[[1-(trifluoromethyl)cyclopropyl]methyl]-2-pyridyl]ethanol (Hydrochloric Acid (2)) (11.7 g, 91%) as white solid. ¹H NMR (300 MHz, DMSO-d₆) δ 9.19 (br. s., 1H), 8.97 (br. s., 1H), 8.59 (d, J=1.8 Hz, 1H), 8.11 (d, J 6.8 Hz, 1H), 7.76 (d, J 8.2 Hz, 1H), 5.17 (dd, J 9.4, 2.9 Hz, 1H), 3.45-3.30 (m, 1H), 3.23-3.08 (m, 1H), 3.05 (s, 2H), 2.60 (t, J 5.4 Hz, 3H), 1.05-0.96 (m, 2H), 0.95-0.86 (m, 2H). ¹⁹F NMR (282 MHz, DMSO-d₆) δ −67.50 (s, 3F). ESI-MS m/z calc. 274.1293, found 275.2 (M+1)⁺; Retention time: 1.75 minutes; LC method G.

### Step 10: 6-(2,6-Dimethylphenyl)-12-methyl-10-(5-{[1-(trifluoromethyl)cyclopropyl]methyl}pyridin-2-yl)-9-oxa-2λ⁶-thia-3,5,12,19-tetraazatricyclo[12.3.1.14,8]nonadeca-1(17),4(19),5,7,14(18),15-hexaene-2,2,13-trione

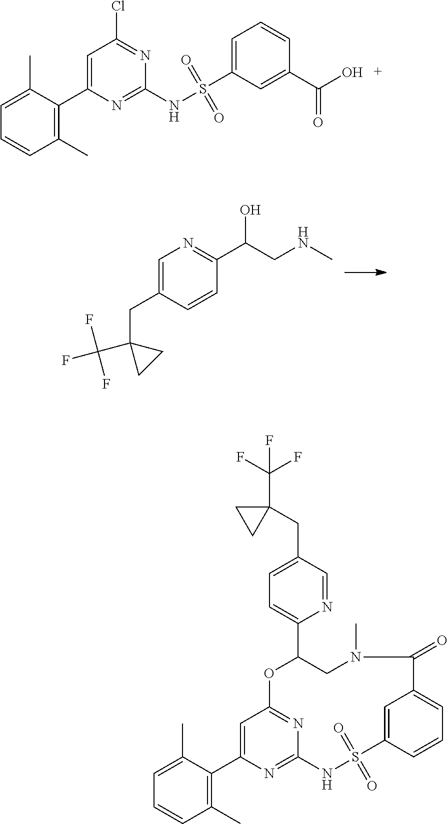 [SMILES: Cc1cccc(C)c1-c1cc(Cl)nc(NS(=O)(=O)c2cccc(C(=O)O)c2)n1 ; CNCC(O)c1ccc(CC2(C(F)(F)F)CC2)cn1 ; Cc1cccc(C)c1-c1cc2nc(n1)NS(=O)(=O)c1cccc(c1)C(=O)N(C)CC(c1ccc(CC3(C(F)(F)F)CC3)cn1)O2]

3-[[4-Chloro-6-(2,6-dimethylphenyl)pyrimidin-2-yl]sulfamoyl]benzoic acid (114 mg, 0.2728 mmol), 2-(methylamino)-1-[5-[[1-(trifluoromethyl)cyclopropyl]methyl]-2-pyridyl]ethanol (dihydrochloride salt) (99.5 mg, 0.2866 mmol), and sodium tert-butoxide (160.6 mg, 1.671 mmol) were combined in THE (3 mL) and stirred at room temperature for 2 h. The reaction mixture was then added dropwise to a stirred solution of HATU (216.2 mg, 0.5686 mmol) in DMF (2.5 mL) and stirred an additional 3 h. The reaction was partitioned between ethyl acetate and a saturated ammonium chloride solution. The organics were separated, washed with brine, dried over sodium sulfate and evaporated. The crude material was purified by reverse-phase HPLC utilizing a gradient of 1-99% acetonitrile in 5 mM aqueous HCl to yield 6-(2,6-dimethylphenyl)-12-methyl-10-(5-{[1-(trifluoromethyl)cyclopropyl]methyl}pyridin-2-yl)-9-oxa-2λ⁶-thia-3,5,12,19-tetraazatricyclo[12.3.1.14,8]nonadeca-1(17),4(19),5,7,14(18),15-hexaene-2,2,13-trione (hydrochloride salt) (63.9 mg, 35%) ESI-MS m/z calc. 637.1971, found 638.2 (M+1)⁺; Retention time: 1.82 minutes; LC method A.

### Step 11: 6-(2,6-Dimethylphenyl)-12-methyl-10-(5-{[1-(trifluoromethyl)cyclopropyl]methyl}pyridin-2-yl)-9-oxa-2×⁶-thia-3,5,12,19-tetraazatricyclo[12.3.1.14,8]nonadeca-1(17),4(19),5,7,14(18),15-hexaene-2,2,13-trione, enantiomer 1 (Compound 104), and 6-(2,6-dimethylphenyl)-12-methyl-10-(5-{[1-(trifluoromethyl)cyclopropyl]methyl}pyridin-2-yl)-9-oxa-2λ⁶-thia-3,5,12,19-tetraazatricyclo[12.3.1.14,8]nonadeca-1(17),4(19),5,7,14(18),15-hexaene-2,2,13-trione, enantiomer 2 (Compound 105)

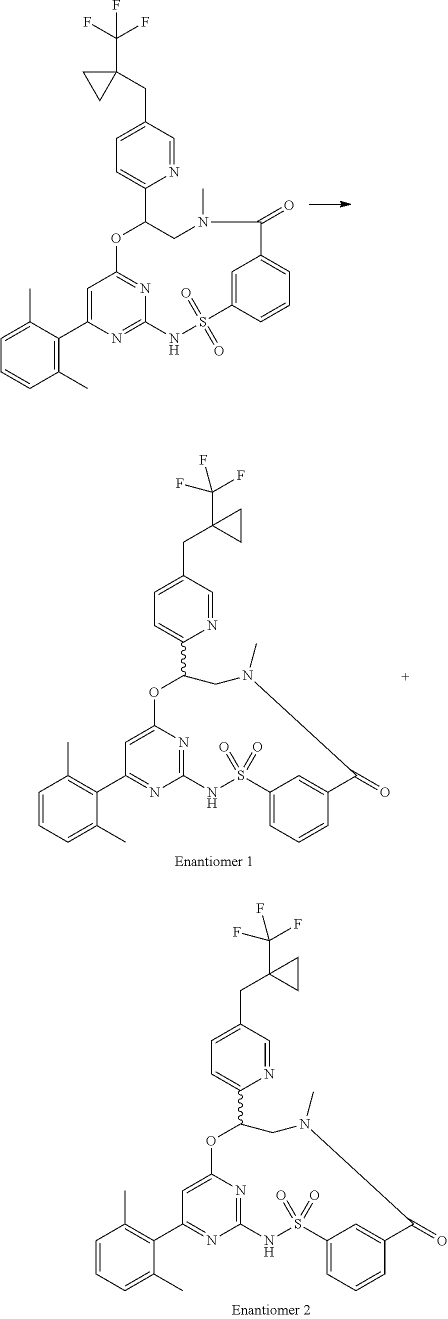 [SMILES: Cc1cccc(C)c1-c1cc2nc(n1)NS(=O)(=O)c1cccc(c1)C(=O)N(C)CC(c1ccc(CC3(C(F)(F)F)CC3)cn1)O2 ; Cc1cccc(C)c1-c1cc2nc(n1)NS(=O)(=O)c1cccc(c1)C(=O)N(C)CC(c1ccc(CC3(C(F)(F)F)CC3)cn1)O2 ; Cc1cccc(C)c1-c1cc2nc(n1)NS(=O)(=O)c1cccc(c1)C(=O)N(C)CC(c1ccc(CC3(C(F)(F)F)CC3)cn1)O2]

Racemic 6-(2,6-dimethylphenyl)-12-methyl-2,2-dioxo-10-[5-[[1-(trifluoromethyl)cyclopropyl]methyl]-2-pyridyl]-9-oxa-2λ⁶-thia-3,5,12,19-tetrazatricyclo[12.3.1.14,8]nonadeca-1(18),4(19),5,7,14,16-hexaen-13-one (hydrochloride salt) (63 mg, 0.09345 mmol) was subjected to SFC separation using a LUX-4 (250×21.2 mm, 5 m) column at 40° C., a mobile phase comprised of 32% MeOH (no modifier) and 68% C02, with a flow of 70 mL/min, at a concentration of 33 mg/mL in MeOH:DMSO (92:8, no modifier), and with an injection volume of 500 μL, and utilizing a pressure of 165 bar and 220/254 nm wavelength to give two enantiomers: Enantiomer 1, peak 1, 6-(2,6-dimethylphenyl)-12-methyl-10-(5-{[1-(trifluoromethyl)cyclopropyl]methyl}pyridin-2-yl)-9-oxa-2λ⁶-thia-3,5,12,19-tetraazatricyclo[12.3.1.14,8]nonadeca-1(17),4(19),5,7,14(18),15-hexaene-2,2,13-trione (28.6 mg, 48%)¹H NMR (400 MHz, DMSO-d₆) δ 13.03 (s, 1H), 8.66-8.56 (m, 2H), 7.98-7.87 (m, 2H), 7.78 (d, J 8.0 Hz, 1H), 7.74-7.57 (m, 2H), 7.29-7.19 (m, 1H), 7.11 (d, J 7.6 Hz, 2H), 6.47 (dd, J 11.0, 4.2 Hz, 1H), 6.33 (s, 1H), 3.80-3.70 (m, 1H), 3.63-3.51 (m, 1H), 3.04 (s, 2H), 2.17 (s, 3H), 2.04 (s, 6H), 1.04-0.97 (m, 2H), 0.97-0.88 (m, 2H). ESI-MS m/z calc. 637.1971, found 638.2 (M+1)⁺; Retention time: 1.69 minutes (LC method A); and enantiomer 2, peak 2 6-(2,6-dimethylphenyl)-12-methyl-10-(5-{[1-(trifluoromethyl)cyclopropyl]methyl}pyridin-2-yl)-9-oxa-2λ⁶-thia-3,5,12,19-tetraazatricyclo[12.3.1.14,8]nonadeca-1(17),4(19),5,7,14(18),15-hexaene-2,2,13-trione (9.7 mg, 16 ESI-MS m/z calc. 637.1971, found 638.2 (M+1)⁺; Retention time: 1.69 minutes; LC method A.

### Example 81: Preparation of Compound 106

### Step 1: tert-Butyl N-benzyl-N-[(2R)-2-hydroxy-3-phenylpropyl]carbamate

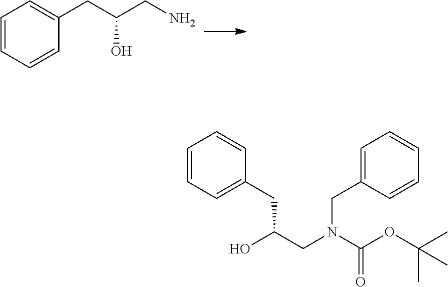 [SMILES: NC[C@H](O)Cc1ccccc1 ; CC(C)(C)OC(=O)N(Cc1ccccc1)C[C@H](O)Cc1ccccc1]

(2R)-1-Amino-3-phenyl-propan-2-ol (302.4 mg, 2 mmol) was dissolved in tetrahydrofuran. Bromomethylbenzene (approximately 393.4 mg, 273.6 μL, 2.300 mmol) was added followed by solid potassium carbonate (approximately 276.4 mg, 2.000 mmol). The reaction mixture was allowed to stir at room temperature for 2 hours. Di-tert-butyl dicarbonate (approximately 436.5 mg, 459.5 μL, 2.000 mmol) was then added. The reaction mixture was allowed to stir overnight at room temperature. The reaction mixture was diluted with DCM (7 mL) and washed with aqueous HCl (1 M, 1×7 mL) and brine (2×7 mL). The organic layer was dried over sodium sulfate. The crude product was chromatographed on a 12 gram silica gel column eluting with a 0-40% EtOAc/hexane gradient over 30 minutes. tert-butyl N-benzyl-N-[(2R)-2-hydroxy-3-phenylpropyl]carbamate (83 mg, 12%) was obtained. ESI-MS m/z calc. 341.1991, found 342.0 (M+1)⁺; Retention time: 1.94 minutes; LC method A.

### Step 2: 3-[(4-{[(2R)-1-{Benzyl[(tert-butoxy)carbonyl]amino}-3-phenylpropan-2-yl]oxy}-6-(2,6-dimethylphenyl)pyrimidin-2-yl)sulfamoyl]benzoic acid

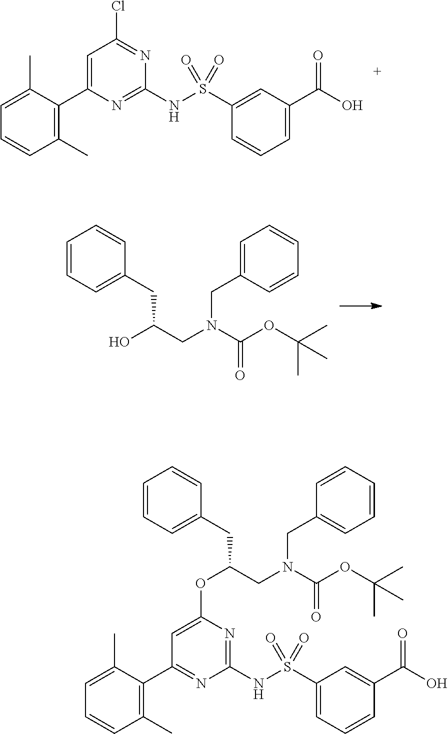 [SMILES: Cc1cccc(C)c1-c1cc(Cl)nc(NS(=O)(=O)c2cccc(C(=O)O)c2)n1 ; CC(C)(C)OC(=O)N(Cc1ccccc1)C[C@H](O)Cc1ccccc1 ; Cc1cccc(C)c1-c1cc(O[C@H](Cc2ccccc2)CN(Cc2ccccc2)C(=O)OC(C)(C)C)nc(NS(=O)(=O)c2cccc(C(=O)O)c2)n1]

3-[[4-Chloro-6-(2,6-dimethylphenyl)pyrimidin-2-yl]sulfamoyl]benzoic acid (approximately 32.62 mg, 0.07807 mmol) was dissolved into a solution of tert-butyl N-benzyl-N-[(2R)-2-hydroxy-3-phenylpropyl]carbamate (approximately 39.98 mg, 0.1171 mmol) in tetrahydrofuran (2 mL). Solid sodium tert-butoxide (approximately 37.51 mg, 0.3903 mmol) was added. The reaction mixture was allowed to stir at room temperature overnight. The reaction mixture was diluted with DCM (7 mL) and washed with aqueous HCl (1 M, 1×7 mL) and brine (2×7 mL). The organic layer was dried over sodium sulfate, filtered and concentrated under reduced pressure. The product was used in the next step without further purification. 3-[(4-{[(2R)-1-{Benzyl[(tert-butoxy)carbonyl]amino}-3-phenylpropan-2-yl]oxy}-6-(2,6-dimethylphenyl)pyrimidin-2-yl)sulfamoyl]benzoic acid (94 mg, 167%) was obtained. ESI-MS m/z calc. 722.2774, found 723.0 (M+1)⁺; Retention time: 2.192 minutes; LC method A.

### Step 3: (10R)-10,12-Dibenzyl-6-(2,6-dimethylphenyl)-9-oxa-2×⁶-thia-3,5,12,19-tetraazatricyclo[12.3.1.14,8]nonadeca-1(17),4(19),5,7,14(18),15-hexaene-2,2,13-trione (Compound 106)

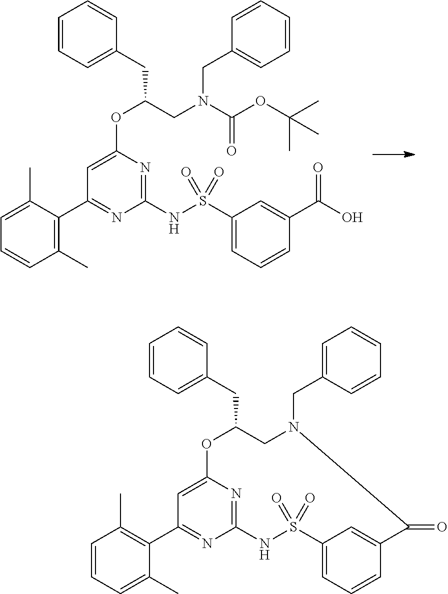 [SMILES: Cc1cccc(C)c1-c1cc(O[C@H](Cc2ccccc2)CN(Cc2ccccc2)C(=O)OC(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccccc1)C[C@@H](Cc1ccccc1)O2]

3-[(4-{[(2R)-1-{Benzyl[(tert-butoxy)carbonyl]amino}-3-phenylpropan-2-yl]oxy}-6-(2,6-dimethylphenyl)pyrimidin-2-yl)sulfamoyl]benzoic acid was dissolved in a solution of HCl in dioxane. The solution was allowed to stir at room temperature for 15 minutes. Volatiles were removed under reduced pressure. The remaining oil was dissolved in DMF (0.7 mL), and HATU was added followed by triethylamine. The reaction mixture was allowed to stir for an additional 15 minutes. After filtration, the product was isolated by reverse-phase HPLC using a Luna Cis (2) column (50×21.2 mm, 5 μm particle size) sold by Phenomenex (pn: 00B-4252-PO-AX), and a dual gradient run from 10-99% mobile phase B over 15.0 minutes. Mobile phase A=water (5 mM acid modifier). Mobile phase B=acetonitrile. Flow rate=35 mL/min, injection volume=950 μL, and column temperature=25° C. The UV trace at 254 nm was used to collect fractions. (10R)-10,12-dibenzyl-6-(2,6-dimethylphenyl)-9-oxa-2λ⁶-thia-3,5,12,19-tetraazatricyclo[12.3.1.14,8]nonadeca-1(17),4(19),5,7,14(18),15-hexaene-2,2,13-trione (4.7 mg) was obtained. ESI-MS m/z calc. 604.2144, found 605.3 (M+1)⁺; Retention time: 2.0 minutes; LC method A.

### Example 82: Preparation of Compound 107

### Step 1: 3-[[4-(2,6-Dimethylphenyl)-6-[(2R)-2-[(5-ethoxy-5-oxo-pentyl)amino]-4,4-dimethyl-pentoxy]pyrimidin-2-yl]sulfamoyl]benzoic acid

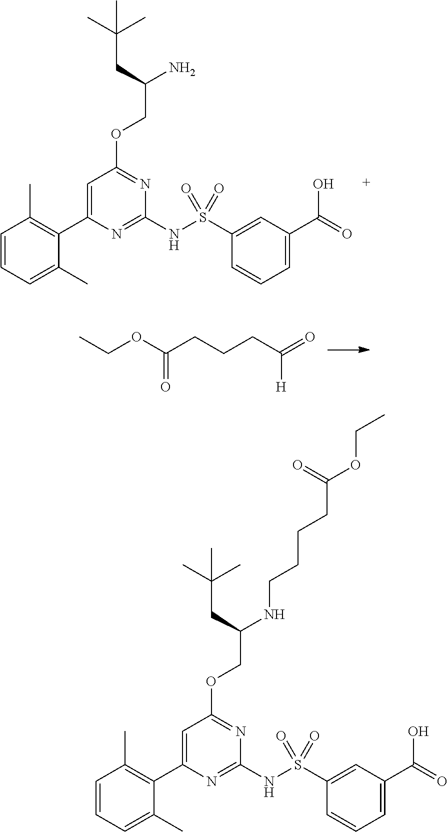

3-[[4-[(2R)-2-Amino-4,4-dimethyl-pentoxy]-6-(2,6-dimethylphenyl)pyrimidin-2-yl]sulfamoyl]benzoic acid (hydrochloride salt) (107.9 mg, 0.1965 mmol), ethyl 5-oxopentanoate (28.4 mg, 0.1970 mmol), and sodium triacetoxyborohydride (211.5 mg, 0.9979 mmol) were combined in DCM (1 mL) and stirred at room temperature for 1 h. The reaction was quenched with methanol, then 1M HCl, and the resulting solution was extracted with ethyl acetate. The organics were washed with brine, dried over sodium sulfate and evaporated. The crude material was purified by reverse phase HPLC utilizing a gradient of 1-70% acetonitrile in 5 mM aqueous HCl to yield 3-[[4-(2,6-dimethylphenyl)-6-[(2R)-2-[(5-ethoxy-5-oxo-pentyl)amino]-4,4-dimethyl-pentoxy]pyrimidin-2-yl]sulfamoyl]benzoic acid (hydrochloride salt) (32 mg, 24%) as a white solid. ESI-MS m/z calc. 640.2931, found 641.9 (M+1)⁺; Retention time: 0.53 minutes; LC method D.

### Step 2: Ethyl 5-[(11R)-6-(2,6-dimethylphenyl)-11-(2,2-dimethylpropyl)-2,2,13-trioxo-9-oxa-2)⁶-thia-3,5,12,19-tetraazatricyclo[12.3.1.14,8]nonadeca-1(18),4,6,8(19),14,16-hexaen-12-yl]pentanoate

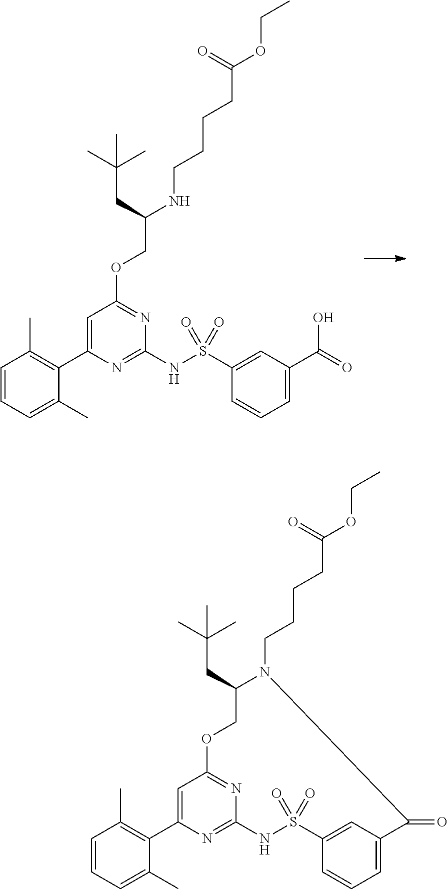 [SMILES: CCOC(=O)CCCCN[C@@H](COc1cc(-c2c(C)cccc2C)nc(NS(=O)(=O)c2cccc(C(=O)O)c2)n1)CC(C)(C)C ; CCOC(=O)CCCCN1C(=O)c2cccc(c2)S(=O)(=O)Nc2nc(cc(-c3c(C)cccc3C)n2)OC[C@H]1CC(C)(C)C]

3-[[4-(2,6-Dimethylphenyl)-6-[(2R)-2-[(5-ethoxy-5-oxo-pentyl)amino]-4,4-dimethyl-pentoxy]pyrimidin-2-yl]sulfamoyl]benzoic acid (hydrochloride salt) (42 mg, 0.06202 mmol) and HATU (29 mg, 0.07627 mmol) were dissolved in DMF (1 mL), then DIEA (54 μL, 0.3100 mmol) was added. The reaction mixture was stirred at room temperature for 1 h. The mixture was filtered and purified by reverse phase HPLC utilizing a gradient of 1-99% acetonitrile in 5 mM aqueous HCl to yield ethyl 5-[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(18),4,6,8(19),14,16-hexaen-12-yl]pentanoate (25.6 mg, 66%) as a white solid. ESI-MS m z calc. 622.28253, found 623.8 (M+1)⁺; Retention time: 0.74 minutes; LC method D.

### Step 3: (11R)-6-(2,6-Dimethylphenyl)-11-(2,2-dimethylpropyl)-12-(5-hydroxy-5-methyl-hexyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 107)

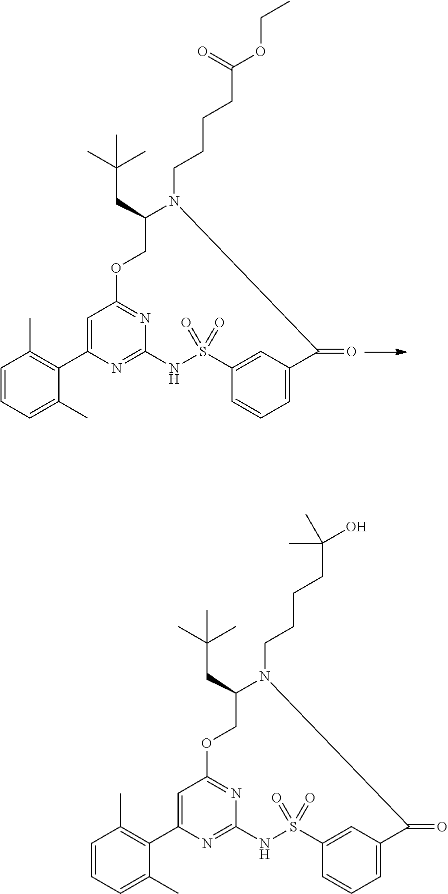 [SMILES: CCOC(=O)CCCCN1C(=O)c2cccc(c2)S(=O)(=O)Nc2nc(cc(-c3c(C)cccc3C)n2)OC[C@H]1CC(C)(C)C ; Cc1cccc(C)c1-c1cc2nc(n1)NS(=O)(=O)c1cccc(c1)C(=O)N(CCCCC(C)(C)O)[C@H](CC(C)(C)C)CO2]

Ethyl 5-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4,6,8(19),14,16-hexaen-12-yl]pentanoate (25.6 mg, 0.04111 mmol) was dissolved in THE (0.5 μL) and bromo(methyl)magnesium (90 μL of 3 M, 0.2700 mmol) was added dropwise. The reaction was stirred at room temperature for 1 h. The reaction was quenched with a saturated ammonium chloride solution and extracted with ethyl acetate. The organics were separated, washed with brine, dried over sodium sulfate and evaporated. The crude material was purified by reverse phase HPLC utilizing a gradient of 1-99% acetonitrile in 5 mM aqueous HCl to yield (11R)-6-(2,6-dimethylphenyl)-11-(2,2-dimethylpropyl)-12-(5-hydroxy-5-methyl-hexyl)-2,2-dioxo-9-oxa-2λ⁶-thia-3,5,12,19-tetrazatricyclo[12.3.1.14,8]nonadeca-1(18),4(19),5,7,14,16-hexaen-13-one (11.7 mg, 47%) as a white solid. ESI-MS m/z calc. 608.3032, found 609.8 (M+1)⁺; Retention time: 1.76 minutes; LC method A.

### Example 83: Preparation of Compound 108

### Step 1: Methyl 4-[2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]ethyl]benzoate

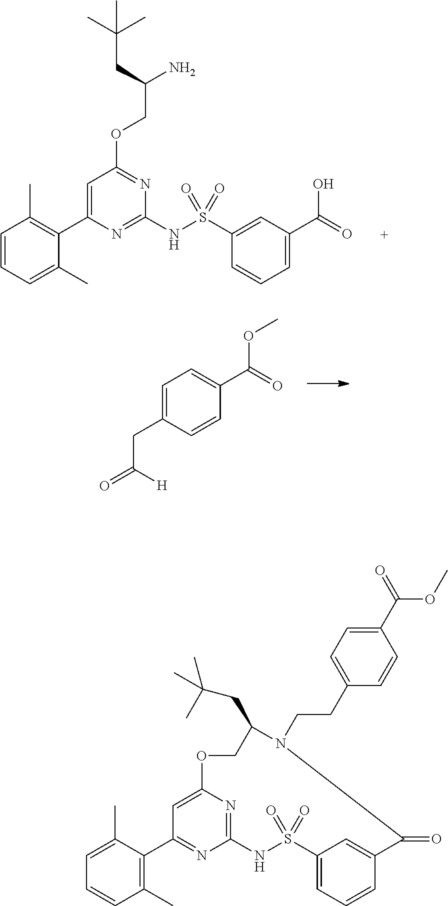

3-[[4-[(2R)-2-Amino-4,4-dimethyl-pentoxy]-6-(2,6-dimethylphenyl)pyrimidin-2-yl]sulfamoyl]benzoic acid (hydrochloride salt) (80 mg, 0.1457 mmol) was combined with the methyl 4-(2-oxoethyl)benzoate (approximately 33.75 mg, 0.1894 mmol) in DCM (0.5 mL) and stirred for 15 minutes at room temperature. Sodium triacetoxyborohydride (approximately 30.88 mg, 0.1457 mmol) was added and the reaction was stirred for an additional 15 minutes. Additional sodium triacetoxyborohydride (approximately 92.64 mg, 0.4371 mmol) was added, and the reaction was stirred for a further 90 minutes at room temperature. The reaction mixture was then partitioned between 1M HCl and ethyl acetate. The layers were separated and the aqueous was extracted 3× ethyl acetate. The combined organics were washed with brine, dried over sodium sulfate and concentrated. The resulting crude material was dissolved in 1:1 DMSO/methanol, filtered and purified by reverse phase HPLC (1-70% ACN in water, HCl modifier, 15 min run) to give the reductive amination product, which was used in the next step without additional purification. The product was dissolved in DMF (1 mL) and added at a rapid dropwise to a stirring solution of HATU (approximately 110.8 mg, 0.2914 mmol) and DIPEA (approximately 113.0 mg, 152.3 μL, 0.8742 mmol) in DMF (6 mL). The reaction was allowed to stir 16 hours at room temperature. The reaction mixture was then concentrated to a volume of less than 1 mL, filtered, and purified by reverse phase HPLC (1-99% ACN in water, HCl modifier, 15 min run) to give methyl 4-[2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]ethyl]benzoate (22 mg, 23%). ESI-MS m/z calc. 656.26685, found 657.6 (M+1)⁺; Retention time: 0.76 minutes; LC method D.

### Step 2: (11R)-6-(2,6-Dimethylphenyl)-11-(2,2-dimethylpropyl)-12-[2-[4-(1-hydroxy-1-methyl-ethyl)phenyl]ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 108)

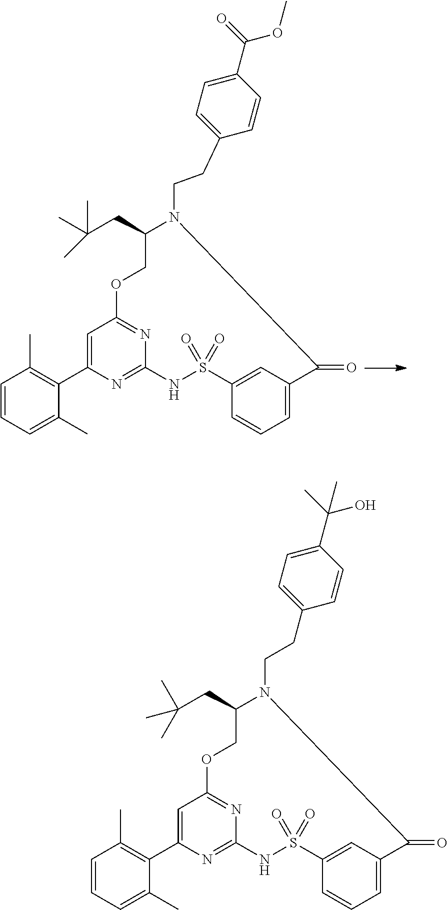 [SMILES: COC(=O)c1ccc(CCN2C(=O)c3cccc(c3)S(=O)(=O)Nc3nc(cc(-c4c(C)cccc4C)n3)OC[C@H]2CC(C)(C)C)cc1 ; Cc1cccc(C)c1-c1cc2nc(n1)NS(=O)(=O)c1cccc(c1)C(=O)N(CCc1ccc(C(C)(C)O)cc1)[C@H](CC(C)(C)C)CO2]

A 4 mL vial was charged under nitrogen with methyl 4-[2-[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]ethyl]benzoate (22 mg, 0.03350 mmol) and anhydrous THE (300 μL) and the solution was cooled down in ice. MeMgBr (70 μL of 3 M, 0.2100 mmol) (3M in diethyl ether) was added dropwise. The reaction was stirred at room temperature for 40 hours. The reaction mixture was cooled to 0° C., quenched with several drops of 1M HCl, diluted with DMSO, and filtered. The resulting material was purified by reverse phase HPLC (1-99% ACN in water, HCl modifier, 15 min run) to give (11R)-6-(2,6-dimethylphenyl)-11-(2,2-dimethylpropyl)-12-[2-[4-(1-hydroxy-1-methyl-ethyl)phenyl]ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (7.3 mg, 33%) ¹H NMR (400 MHz, DMSO-d₆) δ 13.04 (s, 1H), 8.43 (s, 1H), 7.94 (s, 1H), 7.70 (s, 2H), 7.46 (d, J 8.2 Hz, 2H), 7.23 (d, J 8.0 Hz, 3H), 7.11 (s, 2H), 6.36 (s, 1H), 4.91 (s, 1H), 4.50 (s, 1H), 3.96-3.71 (m, 3H), 3.26 (s, 1H), 3.08-2.88 (m, 2H), 2.00 (d, J 76.4 Hz, 6H), 1.65 (dd, J 15.2, 8.2 Hz, 1H), 1.42 (s, 3H), 1.39 (s, 3H), 1.27 (d, J 15.0 Hz, 1H), 0.49 (s, 9H). ESI-MS m/z calc. 656.3032, Retention time: 1.87 minutes (LC method A).

### Example 84: Preparation of Compound 109

### Step 1: tert-Butyl 4-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]piperidine-1-carboxylate

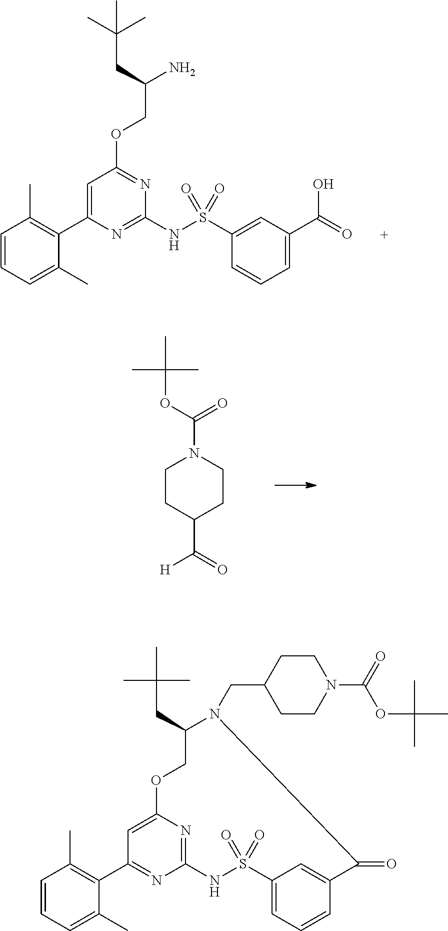 [SMILES: Cc1cccc(C)c1-c1cc(OC[C@H](N)CC(C)(C)C)nc(NS(=O)(=O)c2cccc(C(=O)O)c2)n1 ; CC(C)(C)OC(=O)N1CCC(C=O)CC1 ; Cc1cccc(C)c1-c1cc2nc(n1)NS(=O)(=O)c1cccc(c1)C(=O)N(CC1CCN(C(=O)OC(C)(C)C)CC1)[C@H](CC(C)(C)C)CO2]

3-[[4-[(2R)-2-amino-4,4-dimethyl-pentoxy]-6-(2,6-dimethylphenyl)pyrimidin-2-yl]sulfamoyl]benzoic acid (hydrochloride salt) (250 mg, 0.4553 mmol) was combined with the tert-butyl 4-formylpiperidine-1-carboxylate (approximately 116.5 mg, 0.5464 mmol) in DCM (1 mL) and stirred at room temperature for 15 minutes. Sodium triacetoxyborohydride (approximately 96.50 mg, 0.4553 mmol) (1 equivalent) was added, followed by additional sodium triacetoxyborohydride (approximately 289.5 mg, 1.366 mmol) (3 equivalents) 20 minutes later. The reaction was stirred at room temperature for an additional 30 minutes. The reaction mixture was partitioned between 30 mL 1M HCl and 30 mL ethyl acetate. The layers were separated and the aqueous was extracted an additional 3×20 mL ethyl acetate. The combined organics were washed with brine, dried over sodium sulfate, and concentrated. The resulting crude material was purified by reverse phase HPLC (10-99ACN in water, HCl modifier, 15 min run) to give the reductive amination product after drying. The product was dissolved in DMF (8 mL) and cooled to 0° C. N-methylmorpholine (approximately 276.3 mg, 300.3 μL, 2.732 mmol) was added, followed by CDMT (approximately 103.9 mg, 0.5919 mmol). After 30 minutes the reaction mixture was warmed to room temperature and stirred for an additional 3 hours at room temperature. The reaction mixture was partially concentrated under reduced pressure to a volume of less than 3 mL. The crude material was then filtered and purified by reversed phase HPLC (10-99ACN in water, HCl modifier, 15 min run) to give tert-butyl 4-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]piperidine-1-carboxylate (80 mg, 25%) ESI-MS m/z calc. 691.34033, found 636.8 (M+1)⁺; Retention time: 0.81 minutes; LC method D.

### Step 2: (11R)-6-(2,6-Dimethylphenyl)-11-(2,2-dimethylpropyl)-2,2-dioxo-12-(4-piperidylmethyl)-9-oxa-2λ⁶-thia-3,5,12,19-tetrazatricyclo[12.3.1.14,8]nonadeca-1(18),4(19),5,7,14,16-hexaen-13-one

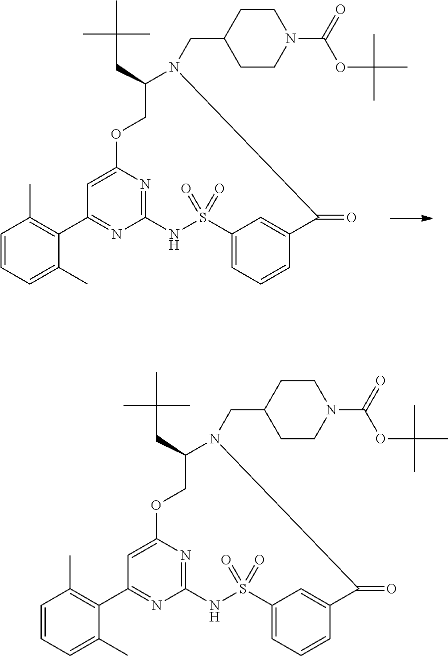 [SMILES: Cc1cccc(C)c1-c1cc2nc(n1)NS(=O)(=O)c1cccc(c1)C(=O)N(CC1CCN(C(=O)OC(C)(C)C)CC1)[C@H](CC(C)(C)C)CO2 ; Cc1cccc(C)c1-c1cc2nc(n1)NS(=O)(=O)c1cccc(c1)C(=O)N(CC1CCN(C(=O)OC(C)(C)C)CC1)[C@H](CC(C)(C)C)CO2]

tert-Butyl 4-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]piperidine-1-carboxylate (80 mg, 0.1156 mmol) was dissolved in DCM (0.5 mL) and HCl (1 mL of 4 M, 4.000 mmol) (in dioxane) was added. The reaction mixture was stirred for 30 minutes at room temperature, then was concentrated under reduced pressure. The resulting product was suspended 2× in DCM/hexanes and again concentrated to give a white solid, (11R)-6-(2,6-dimethylphenyl)-11-(2,2-dimethylpropyl)-2,2-dioxo-12-(4-piperidylmethyl)-9-oxa-2λ⁶-thia-3,5,12,19-tetrazatricyclo[12.3.1.14,8]nonadeca-1(18),4(19),5,7,14,16-hexaen-13-one (hydrochloride salt) (72 mg, 99%), which was used in subsequent steps without further purification. ESI-MS m/z calc. 591.2879, found 592.6 (M+1)⁺; Retention time: 0.53 minutes; LC method D.

### Step 3: (11R)-6-(2,6-Dimethylphenyl)-11-(2,2-dimethylpropyl)-2,2-dioxo-12-[(1-pyrimidin-2-yl-4-piperidyl)methyl]-9-oxa-2λ⁶-thia-3,5,12,19-tetrazatricyclo[12.3.1.14,8]nonadeca-1(18),4(19),5,7,14,16-hexaen-13-one (Compound 109)

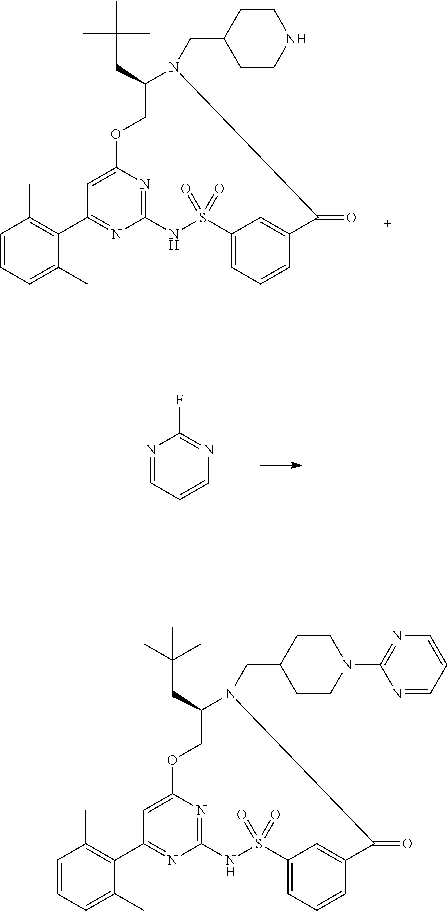 [SMILES: Cc1cccc(C)c1-c1cc2nc(n1)NS(=O)(=O)c1cccc(c1)C(=O)N(CC1CCNCC1)[C@H](CC(C)(C)C)CO2 ; Fc1ncccn1 ; Cc1cccc(C)c1-c1cc2nc(n1)NS(=O)(=O)c1cccc(c1)C(=O)N(CC1CCN(c3ncccn3)CC1)[C@H](CC(C)(C)C)CO2]

The (11R)-6-(2,6-Dimethylphenyl)-11-(2,2-dimethylpropyl)-2,2-dioxo-12-(4-piperidylmethyl)-9-oxa-2λ⁶-thia-3,5,12,19-tetrazatricyclo[12.3.1.14,8]nonadeca-1(18),4(19),5,7,14,16-hexaen-13-one (hydrochloride salt) (15 mg, 0.02388 mmol) was combined with the 2-fluoropyrimidine (5 mg, 0.05098 mmol) and potassium carbonate (approximately 13.20 mg, 0.09552 mmol) in DMF (0.2 mL) and heated to 115° C. until completion. The reaction mixture was then cooled to room temperature, diluted slightly with methanol and filtered, then purified by reverse phase HPLC (1-70% ACN in water, HCl modifier, 15 min run) to give (11R)-6-(2,6-dimethylphenyl)-11-(2,2-dimethylpropyl)-2,2-dioxo-12-[(1-pyrimidin-2-yl-4-piperidyl)methyl]-9-oxa-2λ⁶-thia-3, 5,12,19-tetrazatricyclo[12.3.1.14,8]nonadeca-1(18),4(19),5,7,14,16-hexaen-13-one (6.3 mg, 39%) on drying. ESI-MS m/z calc. 669.30975, found 670.4 (M+1)⁺; Retention time: 1.59 minutes; LC method A.

### Example 85: Preparation of Compound 110

### Step 1: 2-(6-Fluoropyridin-2-yl)acetaldehyde

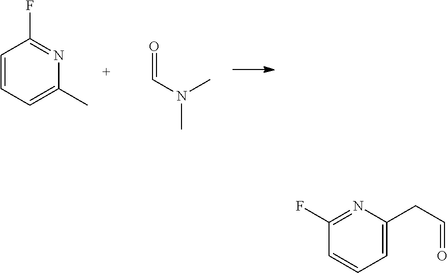

2-Fluoro-6-methyl-pyridine (300 mg, 2.700 mmol) derivative was dissolved in THE (16 mL) and cooled to −78° C. in a dry ice/acetone bath. LDA (approximately 1.755 mL of 2 M, 3.510 mmol) (in THF/heptanes/ethylbenzene) was added dropwise via syringe, and stirring was continued at −78° C. for 1 hour. N,N-dimethylformamide (approximately 1.184 g, 1.254 mL, 16.20 mmol) was then added dropwise by syringe. Stirring was continued at −78° C. for an additional 2 hours, then the reaction mixture was transferred to an ice water bath and allowed to gradually warm from 0° C. to room temperature as the ice melted over an additional 2 hours. The reaction was then returned to 0° C. and quenched by dropwise addition of 5 mL aqueous sodium bicarbonate. The reaction mixture was warmed to room temperature, diluted with water, and extracted 3× ethyl acetate (20 mL ea.) The combined organics were dried over sodium sulfate and concentrated, then immediately dissolved in dichloromethane and purified by chromatography on silica gel (0-10% dichloromethane/methanol). Fractions containing product were combined and concentrated to give 2-(6-fluoropyridin-2-yl)acetaldehyde (180 mg, 48%), which was used immediately in the next step. ESI-MS m/z calc. 139.04333, found 140.0 (M+1)⁺; Retention time: 0.24 minutes; (LC method D).

### Step 2: (11R)-6-(2,6-Dimethylphenyl)-11-(2,2-dimethylpropyl)-12-[2-(6-fluoro-2-pyridyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one

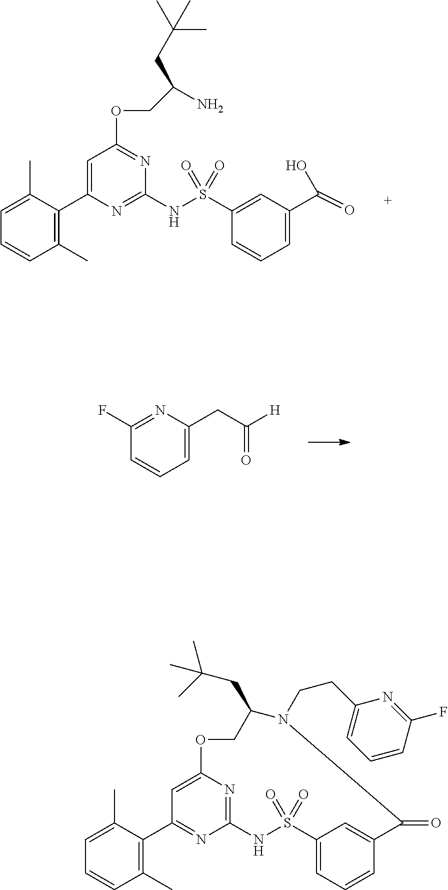 [SMILES: Cc1cccc(C)c1-c1cc(OC[C@H](N)CC(C)(C)C)nc(NS(=O)(=O)c2cccc(C(=O)O)c2)n1 ; O=CCc1cccc(F)n1 ; Cc1cccc(C)c1-c1cc2nc(n1)NS(=O)(=O)c1cccc(c1)C(=O)N(CCc1cccc(F)n1)[C@H](CC(C)(C)C)CO2]

3-[[4-[(2R)-2-Amino-4,4-dimethyl-pentoxy]-6-(2,6-dimethylphenyl)pyrimidin-2-yl]sulfamoyl]benzoic acid (550 mg, 1.073 mmol) (approximately 179.2 mg, 1.288 mmol) in DCM (2 mL), and stirred for 15 minutes at room temperature. Sodium triacetoxyborohydride (approximately 227.4 mg, 1.073 mmol) (1 equiv) was added, followed by additional sodium triacetoxyborohydride (approximately 682.2 mg, 3.219 mmol) (3 equiv) after an additional 15 minutes. The reaction mixture was stirred at room temperature for an additional 2.5 hours, then quenched with 0.4 mL 1M HCl and diluted with 1:1 DMSO methanol until the reaction mixture became homogenous. The reaction mixture was then filtered and purified by reverse phase HPLC (1-70% ACN in water, HCl modifier, 15 min run) to give the reductive amination product upon drying. The product was combined with CDMT (approximately 26.37 mg, 0.1502 mmol) in DMF (1.5 mL), and N-methylmorpholine (approximately 86.82 mg, 94.37 μL, 0.8584 mmol) was added. The reaction mixture was stirred for 3 hours at room temperature, then partially concentrated by rotary evaporation. The crude material was dissolved in 1:1 DMSO/methanol, filtered, and purified by reverse phase HPLC (1-99% ACN in water, HCl modifier, 15 min run) to give as a white solid (11R)-6-(2,6-Dimethylphenyl)-11-(2,2-dimethylpropyl)-12-[2-(6-fluoro-2-pyridyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (42 mg, 6%) ESI-MS m/z calc. 617.2472, found 618.3 (M+1)⁺; Retention time: 0.74 minutes; LC method D.

### Step 3: (11R)-6-(2,6-Dimethylphenyl)-11-(2,2-dimethylpropyl)-12-[2-(6-morpholino-2-pyridyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 110)

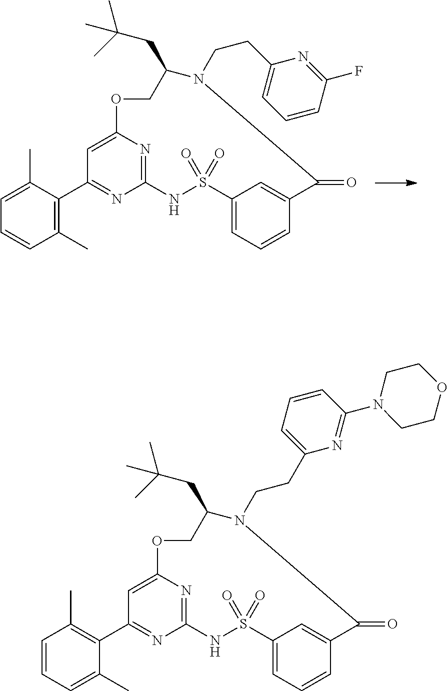 [SMILES: Cc1cccc(C)c1-c1cc2nc(n1)NS(=O)(=O)c1cccc(c1)C(=O)N(CCc1cccc(F)n1)[C@H](CC(C)(C)C)CO2 ; Cc1cccc(C)c1-c1cc2nc(n1)NS(=O)(=O)c1cccc(c1)C(=O)N(CCc1cccc(N3CCOCC3)n1)[C@H](CC(C)(C)C)CO2]

(11R)-6-(2,6-dimethylphenyl)-11-(2,2-dimethylpropyl)-12-[2-(6-fluoro-2-pyridyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (15 mg, 0.02428 mmol), morpholine (approximately 10.58 mg, 10.59 μL, 0.1214 mmol), and potassium carbonate (approximately 26.84 mg, 0.1942 mmol) were combined in DMSO (0.3 mL) and heated to 120° C. for 20 hours. The reaction mixture was then cooled to room temperature, diluted with methanol, filtered, and purified by reverse phase HPLC (1-70% ACN in water, HCl modifier, 15 min run) to give after drying, (11R)-6-(2,6-dimethylphenyl)-11-(2,2-dimethylpropyl)-12-[2-(6-morpholino-2-pyridyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (8 mg, 48%). ESI-MS m/z calc. 684.3094, found 685.4 (M+1)⁺; Retention time: 1.46 minutes; LC method A.

### Example 86: Preparation of Compound 111

### Step 1: 6-Morpholinopyridazine-3-carbaldehyde

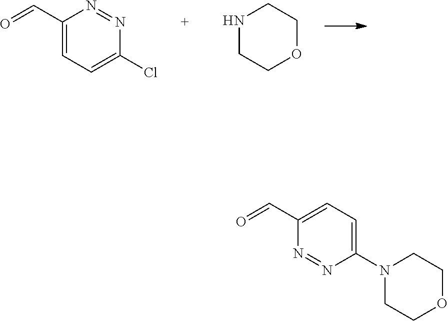 [SMILES: O=Cc1ccc(Cl)nn1 ; C1COCCN1 ; O=Cc1ccc(N2CCOCC2)nn1]

6-Chloropyridazine-3-carbaldehyde (100 mg, 0.7015 mmol) was combined with morpholine (approximately 122.2 mg, 122.3 μL, 1.403 mmol) and potassium carbonate (approximately 387.8 mg, 2.806 mmol) in DMF (2.5 mL). The reaction was stirred at 115° C. for the indicated time. After cooling to room temperature, the reaction mixture was concentrated by rotary evaporation and the resulting crude mixture was purified by chromatography on silica gel to give the indicated 6-morpholinopyridazine-3-carbaldehyde (109 mg, 80%). ESI-MS m/z calc. 193.08513, found 194.2 (M+1)⁺; Retention time: 0.24 minutes; (LC method D).

### Step 2: (11R)-6-(2,6-Dimethylphenyl)-11-(2,2-dimethylpropyl)-12-[(6-morpholinopyridazin-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 111)

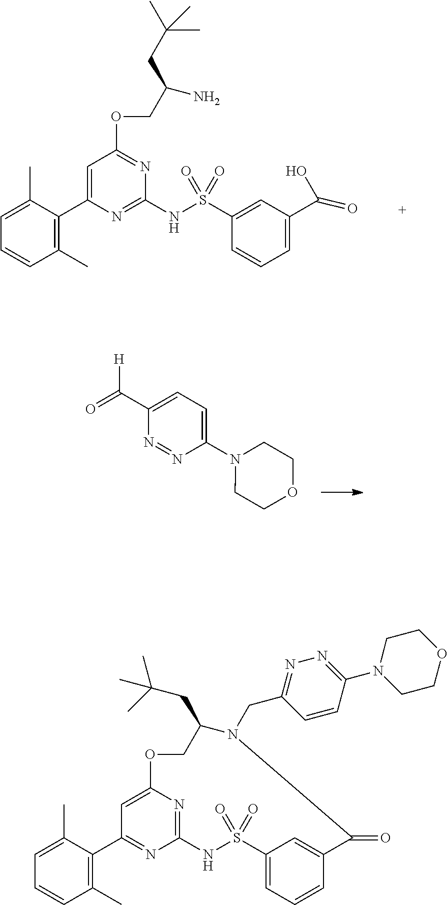 [SMILES: Cc1cccc(C)c1-c1cc(OC[C@H](N)CC(C)(C)C)nc(NS(=O)(=O)c2cccc(C(=O)O)c2)n1 ; O=Cc1ccc(N2CCOCC2)nn1 ; Cc1cccc(C)c1-c1cc2nc(n1)NS(=O)(=O)c1cccc(c1)C(=O)N(Cc1ccc(N3CCOCC3)nn1)[C@H](CC(C)(C)C)CO2]

3-[[4-[(2R)-2-Amino-4,4-dimethyl-pentoxy]-6-(2,6-dimethylphenyl)pyrimidin-2-yl]sulfamoyl]benzoic acid (50 mg, 0.09754 mmol) was combined with 6-morpholinopyridazine-3-carbaldehyde (approximately 22.60 mg, 0.1170 mmol) in DCM (0.3 mL), and stirred for 15 minutes at room temperature. Sodium triacetoxyborohydride (approximately 20.67 mg, 0.09754 mmol) (1 equivalent) was added, followed by additional sodium triacetoxyborohydride (approximately 62.01 mg, 0.2926 mmol) (3 equivalent) after an additional 15 minutes. The reaction mixture was stirred at room temperature for an additional 2.5 hours, then quenched with 0.4 mL 1M HCl and diluted with 1:1 DMSO methanol until the reaction mixture became homogenous. The reaction mixture was then filtered and purified by reverse phase HPLC (1-70% ACN in water, HCl modifier, 15 min run) to give the reductive amination product upon drying. The product was combined with CDMT (approximately 5.652 mg, 0.03219 mmol) in DMF (1.5 mL), and N-methylmorpholine (approximately 24.67 mg, 26.82 μL, 0.2438 mmol) was added. The reaction was stirred at room temperature 3 hours. The reaction mixture was then filtered and purified by reverse phase HPLC (1-99% ACN in water, HCl modifier, 15 min run) to give upon drying (11R)-6-(2,6-dimethylphenyl)-11-(2,2-dimethylpropyl)-12-[(6-morpholinopyridazin-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (6.7 mg, 10%). ESI-MS m/z calc. 671.289, found 672.5 (M+1)⁺; Retention time: 1.4 minutes; LC method A.

### Example 87: Preparation of Compound 112

### Step 1: 2-(5-Bromopyridin-2-yl)acetaldehyde

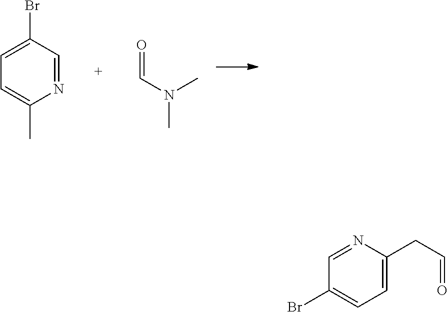

5-Bromo-2-methyl-pyridine (300 mg, 1.744 mmol) derivative was dissolved in THE (12 mL) and cooled to −78° C. in a dry ice/acetone bath. LDA (approximately 1.134 mL of 2 M, 2.267 mmol) (in THF/heptanes/ethylbenzene) was added dropwise via syringe, and stirring was continued at −78° C. for 1 hour. N,N-dimethylformamide (approximately 764.6 mg, 810.0 μL, 10.46 mmol) was then added dropwise by syringe. Stirring was continued at −78° C. for an additional 2 hours, then the reaction mixture was transferred to an ice water bath and allowed to gradually warm from 0° C. to room temperature as the ice melted over an additional 2 hours. The reaction was then returned to 0° C. and quenched by dropwise addition of 5 mL aqueous sodium bicarbonate. The reaction mixture was warmed to room temperature, diluted with water, and extracted 3× ethyl acetate (20 mL ea.) The combined organics were dried over sodium sulfate and concentrated, then immediately dissolved in dichloromethane and purified by chromatography on silica gel (0-10% dichloromethane/methanol). Fractions containing product were combined and concentrated to give 2-(5-bromopyridin-2-yl)acetaldehyde (35 mg, 10%), which was used immediately in the next step. ESI-MS m/z calc. 198.96327, found 199.9 (M+1)⁺; Retention time: 0.27 minutes (LC method D).

### Step 2: (11R)-12-[2-(5-Bromo-2-pyridyl)ethyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

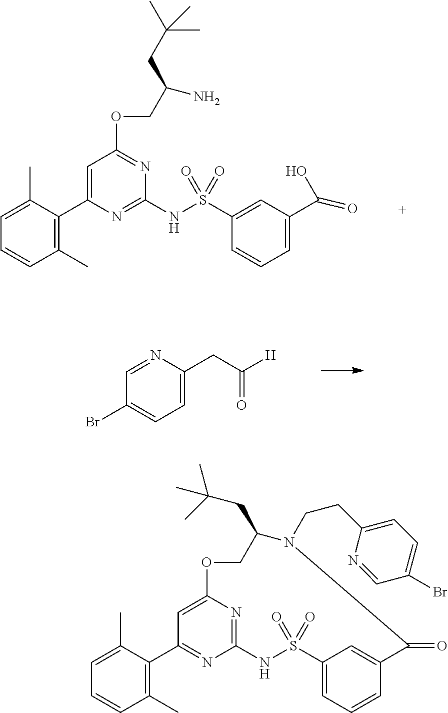 [SMILES: Cc1cccc(C)c1-c1cc(OC[C@H](N)CC(C)(C)C)nc(NS(=O)(=O)c2cccc(C(=O)O)c2)n1 ; O=CCc1ccc(Br)cn1 ; Cc1cccc(C)c1-c1cc2nc(n1)NS(=O)(=O)c1cccc(c1)C(=O)N(CCc1ccc(Br)cn1)[C@H](CC(C)(C)C)CO2]

3-[[4-[(2R)-2-Amino-4,4-dimethyl-pentoxy]-6-(2,6-dimethylphenyl)pyrimidin-2-yl]sulfamoyl]benzoic acid (70 mg, 0.1366 mmol) was combined with 2-(5-bromopyridin-2-yl)acetaldehyde (35 mg, 0.1750 mmol) in DCM (0.5 mL), and stirred for 15 minutes at room temperature. Sodium triacetoxyborohydride (approximately 28.95 mg, 0.1366 mmol) (1 equivalent) was added, followed by additional sodium triacetoxyborohydride (approximately 86.85 mg, 0.4098 mmol) (3 equivalent) after an additional 15 minutes. The reaction mixture was stirred at room temperature for an additional 2.5 hours, then quenched with 0.4 mL 1M HCl and diluted with 1:1 DMSO methanol until the reaction mixture became homogenous. The reaction mixture was then filtered and purified by reverse phase HPLC (1-70% ACN in water, HCl modifier, 15 min run) to give the reductive amination product upon drying. The product was combined with CDMT (approximately 9.593 mg, 0.05464 mmol) in DMF (1.5 mL), and N-methylmorpholine (approximately 30.39 mg, 33.03 μL, 0.3005 mmol) was added. The reaction was stirred at room temperature for 3 hours. After this point the reaction was filtered and purified by reverse phase HPLC (1-99% ACN in water HCl modifier, 15 min run) to give upon drying the (11R)-12-[2-(5-bromo-2-pyridyl)ethyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (14 mg, 15%). ESI-MS m/z calc. 677.1671, found 680.4 (M+3)⁺; Retention time: 0.75 minutes; LC method D.

### Step 3: (11R)-12-[2-(5-Cyclohexyl-2-pyridyl)ethyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 112)

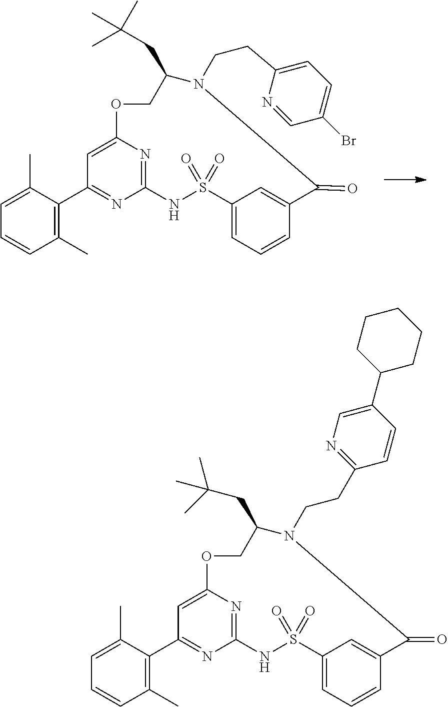 [SMILES: Cc1cccc(C)c1-c1cc2nc(n1)NS(=O)(=O)c1cccc(c1)C(=O)N(CCc1ccc(Br)cn1)[C@H](CC(C)(C)C)CO2 ; Cc1cccc(C)c1-c1cc2nc(n1)NS(=O)(=O)c1cccc(c1)C(=O)N(CCc1ccc(C3CCCCC3)cn1)[C@H](CC(C)(C)C)CO2]

(11R)-12-[2-(5-bromo-2-pyridyl)ethyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (14 mg, 0.02063 mmol), cyclohexen-1-ylboronic acid (4.5 mg, 0.03573 mmol), Pd(dppf)Cl₂ (1 mg, 0.001225 mmol), and potassium carbonate (7.5 mg, 0.05427 mmol) in a screwcap vial, which was then purged with nitrogen. DMSO (200 μL) and water (50 μL) were added by syringe, and the reaction mixture was heated to 100° C. for 90 minutes. The reaction mixture was then cooled to room temperature, filtered, and purified by reverse phase HPLC (1-70% ACN in water, HCl modifier, 15 min run) to give (11R)-12-[2-[5-(cyclohexen-1-yl)-2-pyridyl]ethyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (9.3 mg, 66%) ESI-MS m/z calc. 679.3192, found 680.4 (M+1)⁺; Retention time: 0.64 minutes; LC method D.

The product from above was combined with dihydroxypalladium (3 mg, 0.002136 mmol) on charcoal in a screwcap vial, which was then purged with nitrogen. Methanol (1 mL) was added, and hydrogen gas was bubbled through the reaction mixture from a balloon for 30 minutes. The reaction mixture was then stirred for an additional 30 minutes with the hydrogen balloon in place. After this time the reaction vessel was purged with nitrogen, and the reaction mixture was filtered and purified by reverse phase HPLC (1-99% ACN in water, HCl modifier, 15 min run) to give upon drying (11R)-12-[2-(5-cyclohexyl-2-pyridyl)ethyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (5.4 mg, 36%). ESI-MS m/z calc. 681.3349, found 682.4 (M+1)⁺; Retention time: 1.65 minutes; LC method A.

### Example 88: Preparation of Compound 113

### Step 1: 3-[[4-(2,6-Dimethylphenyl)-6-[(2R)-4,4-dimethyl-2-(pyrimidin-2-ylmethylamino)pentoxy]pyrimidin-2-yl]sulfamoyl]benzoic acid

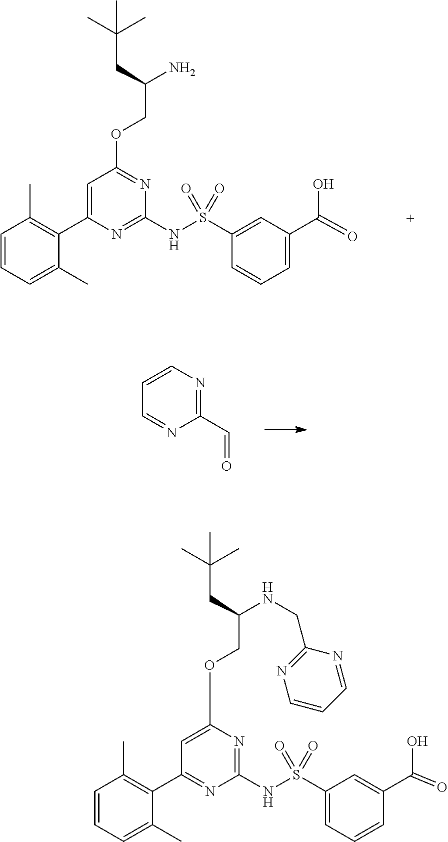 [SMILES: Cc1cccc(C)c1-c1cc(OC[C@H](N)CC(C)(C)C)nc(NS(=O)(=O)c2cccc(C(=O)O)c2)n1 ; O=Cc1ncccn1 ; Cc1cccc(C)c1-c1cc(OC[C@@H](CC(C)(C)C)NCc2ncccn2)nc(NS(=O)(=O)c2cccc(C(=O)O)c2)n1]

3-[[4-[(2R)-2-amino-4,4-dimethyl-pentoxy]-6-(2,6-dimethylphenyl)pyrimidin-2-yl]sulfamoyl]benzoic acid (hydrochloride salt) (30 mg, 0.05464 mmol), pyrimidine-2-carbaldehyde (approximately 5.906 mg, 0.05464 mmol), acetic acid (approximately 3.609 mg, 3.418 μL, 0.06010 mmol), and triethylamine (approximately 6.082 mg, 8.377 μL, 0.06010 mmol) were combined in dichloromethane (0.5 mL) and stirred for 5 min. Sodium triacetoxyborohydride (approximately 17.37 mg, 0.08196 mmol) was added and the reaction mixture was stirred an additional 1 h. The reaction was quenched with methanol (0.5 mL) and a 1M HCl solution (0.1 mL), then evaporated. The crude material was purified by reverse phase HPLC utilizing a gradient of 1-99% acetonitrile in 5 mM aqueous HCl to yield 3-[[4-(2,6-dimethylphenyl)-6-[(2R)-4,4-dimethyl-2-(pyrimidin-2-ylmethylamino)pentoxy]pyrimidin-2-yl]sulfamoyl]benzoic acid (hydrochloride salt) (18.1 mg, 55%). ESI-MS m/z calc. 604.24677, found 605.1 (M+1)⁺; Retention time: 0.47 minutes; LC method D.

### Step 2: (11R)-6-(2,6-Dimethylphenyl)-11-(2,2-dimethylpropyl)-12-[(pyrimidin-2-yl)methyl]-9-oxa-2λ⁶-thia-3,5,12,19-tetraazatricyclo[12.3.1.14,8]nonadeca-1(17),4(19),5,7,14(18),15-hexaene-2,2,13-trione (Compound 113)

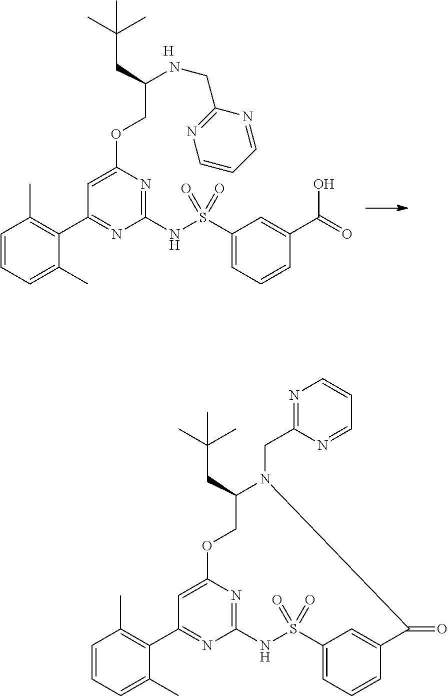

3-[[4-(2,6-Dimethylphenyl)-6-[(2R)-4,4-dimethyl-2-(pyrimidin-2-ylmethylamino)pentoxy]pyrimidin-2-yl]sulfamoyl]benzoic acid (hydrochloride salt) (18 mg, 0.02807 mmol), 4-(6-cyano-2-methyl-7-oxo-4,8-dioxa-2,5-diazadec-5-en-3-ylidene)morpholin-4-ium hexafluorophosphate(V) (18.1 mg, 0.04226 mmol), and DIEA (20 μL, 0.1148 mmol) were combined in DMF (1 mL) and stirred at room temperature for 6 h. The reaction was filtered and purified by reverse phase HPLC utilizing a gradient of 1-99% acetonitrile in 5 mM aqueous HCl to yield (11R)-6-(2,6-dimethylphenyl)-11-(2,2-dimethylpropyl)-12-[(pyrimidin-2-yl)methyl]-9-oxa-2λ⁶-thia-3,5,12,19-tetraazatricyclo[12.3.1.14,8]nonadeca-1(17),4(19),5,7,14(18),15-hexaene-2,2,13-trione (5.5 mg, 33%). ESI-MS m/z calc. 586.2362, found 587.8 (M+1)⁺; Retention time: 1.63 minutes; LC method A.

### Example 89: Preparation of Compound 114

### Step 1: 5-[2-Methoxyethyl(methyl)amino]pyrimidine-2-carbaldehyde

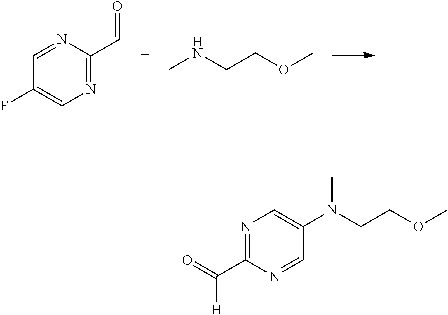

To a stirred solution of 2-methoxy-N-methyl-ethanamine (63 mg, 0.7068 mmol) in anhydrous DMF (1.5 mL) was added 5-fluoropyrimidine-2-carbaldehyde (80 mg, 0.6345 mmol), followed by addition of cesium carbonate (312 mg, 0.9576 mmol). The heterogenous mixture was purged with nitrogen briefly, then stirred at 110° C. for 15 h (overnight). The mixture was allowed to cool to ambient temperature. The dark reaction mixture was poured over ice-water (15 mL) and extracted with ethyl acetate (2×15 mL).The combined organics were washed sequentially with water (15 mL), brine (15 mL), dried over sodium sulphate, filtered and concentrated under reduced pressure to furnish 5-[2-methoxyethyl(methyl)amino]pyrimidine-2-carbaldehyde (56 mg, 45%) as a tan solid. It was used in the subsequent reaction without further purification. ESI-MS m/z calc. 195.10078, found 196.1 (M+1)⁺; Retention time: 0.83 minutes; (LC method A). ¹H NMR (400 MHz, DMSO-d₆) δ 9.75 (s, 1H), 8.45 (s, 2H), 3.72 (t, J 5.3 Hz, 2H), 3.55 (t, J 5.2 Hz, 2H), 3.25 (s, 3H), 3.10 (s, 3H).

### Step 2: 3-[[4-(2,6-Dimethylphenyl)-6-[(2R)-2-[[5-[2-methoxyethyl(methyl)amino]pyrimidin-2-yl]methylamino]-4,4-dimethyl-pentoxy]pyrimidin-2-yl]sulfamoyl]benzoic acid

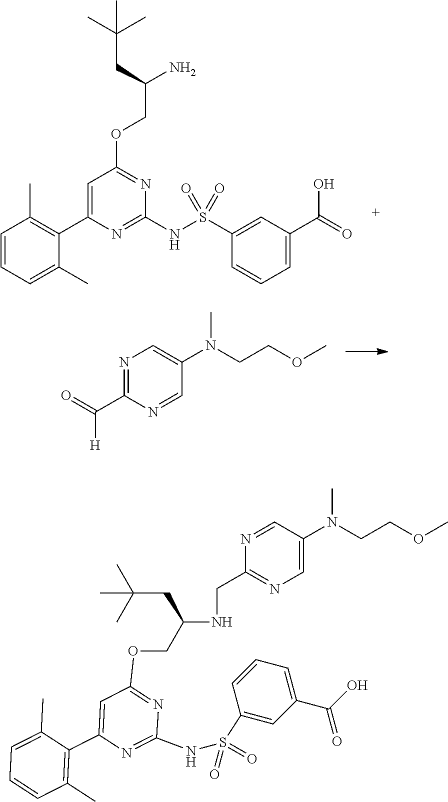 [SMILES: Cc1cccc(C)c1-c1cc(OC[C@H](N)CC(C)(C)C)nc(NS(=O)(=O)c2cccc(C(=O)O)c2)n1 ; COCCN(C)c1cnc(C=O)nc1 ; COCCN(C)c1cnc(CN[C@@H](COc2cc(-c3c(C)cccc3C)nc(NS(=O)(=O)c3cccc(C(=O)O)c3)n2)CC(C)(C)C)nc1]

In a 4 mL vial, to a stirred heterogeneous mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (50 mg, 0.09106 mmol) in anhydrous dichloromethane (0.6 mL) were added 5-[2-methoxyethyl(methyl)amino]pyrimidine-2-carbaldehyde (18 mg, 0.09220 mmol), and glacial acetic acid (10 μL, 0.1758 mmol), in that order. The vial was purged with nitrogen briefly and capped and stirred for 5 min, then sodium triacetoxyborohydride (100 mg, 0.4718 mmol) was added, followed by addition of DIEA (50 μL, 0.2871 mmol), and the capped vial was allowed to stir at ambient temperature for 15 min. Then methanol (0.3 mL) and water (0.2 mL) were added to the reaction and the volatiles were removed under reduced pressure and the residue was taken up in DMSO (1.5 mL), and purified by (reverse-phase HPLC, 1-99% acetonitrile in water over 15 min, HCl as modifier) to furnish 3-[[4-(2,6-dimethylphenyl)-6-[(2R)-2-[[5-[2-methoxyethyl(methyl)amino]pyrimidin-2-yl]methylamino]-4,4-dimethyl-pentoxy]pyrimidin-2-yl]sulfamoyl]benzoic acid (Dihydrochloride salt) (10 mg, 14%) as a white solid. It was used in the subsequent reaction. ESI-MS m/z calc. 691.3152, found 692.4 (M+1)⁺; Retention time: 1.24 minutes; LC method A.

### Step 3: (11R)-6-(2,6-Dimethylphenyl)-11-(2,2-dimethylpropyl)-12-[[5-[2-methoxyethyl(methyl)amino]pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 114)

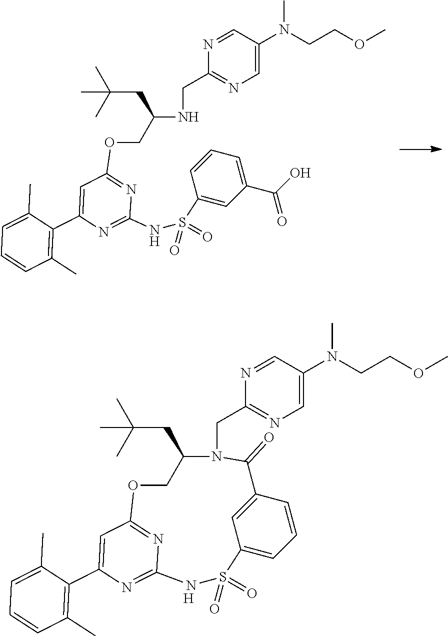

In a 4 mL vial, to a stirred solution of 3-[[4-(2,6-dimethylphenyl)-6-[(2R)-2-[[5-[2-methoxyethyl(methyl)amino]pyrimidin-2-yl]methylamino]-4,4-dimethyl-pentoxy]pyrimidin-2-yl]sulfamoyl]benzoic acid (Dihydrochloride salt) (8 mg, 0.01046 mmol) in anhydrous DMF (1.0 mL) was added [dimethylamino(triazolo[4,5-b]pyridin-3-yloxy)methylene]-dimethyl-ammonium (Phosphorus Hexafluoride Ion) (10 mg, 0.02630 mmol) (HATU), followed by addition of DIEA (10 μL, 0.05741 mmol) at ambient temperature. The vial was briefly purged with nitrogen and the capped vial was allowed to stir at ambient temperature for 2 h. The orange solution was purified by preparative reverse-phase HPLC (1-99% acetonitrile in water over 15 min, 5 mM HCl as modifier), to give (11R)-6-(2,6-dimethylphenyl)-11-(2,2-dimethylpropyl)-12-[[5-[2-methoxyethyl(methyl)amino]pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (4 mg, 53%) as a white solid. ESI-MS m/z calc. 673.3046, found 674.4 (M+1)⁺; Retention time: 1.67 minutes; LC method A.

### Example 90: Preparation of Compound 115

### Step 1: 5-[2,2-Dimethylpropyl(methyl)amino]pyrimidine-2-carbonitrile

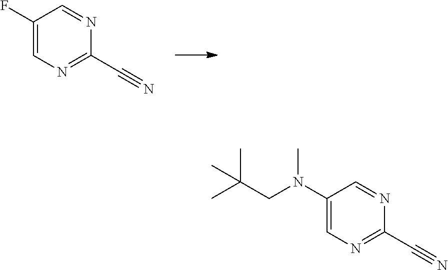

5-Fluoropyrimidine-2-carbonitrile (3.5 g, 27.298 mmol) was dissolved in anhydrous DMF (25 mL) under nitrogen at RT. N,2,2-trimethylpropan-1-amine (hydrochloride salt) (5.1 g, 36.309 mmol) was added to it followed by Cesium carbonate (18.3 g, 56.166 mmol). The reaction mixture was then stirred at 50° C. for 2 h. The reaction mixture was then poured into ice-cold water (200 ml) and stirred for 5 min, and ethyl-acetate (300 ml) was then added to it. The aqueous solution was then extracted with ethyl-acetate (3×300 ml). The combined organic solution was then washed with saturated lithium chloride solution (150 ml) and the organic solution was then dried over anhydrous sodium sulfate, filtered. The solvent was removed by rotary evaporation to furnish 5-[2,2-dimethylpropyl(methyl)amino]pyrimidine-2-carbonitrile (5 g, 81%) as light yellow solid. The product was used in the next step without further purification. ESI-MS m/z calc. 204.1375, found 205.3 (M+1)⁺; Retention time: 4.83 minutes; LC method S.

### Step 2: 5-[2,2-Dimethylpropyl(methyl)amino]pyrimidine-2-carbaldehyde

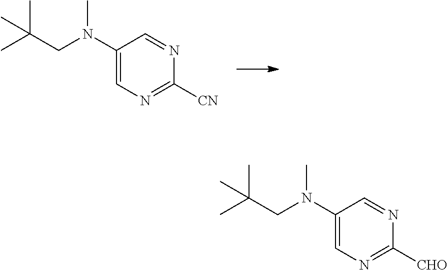

5-[2,2-Dimethylpropyl(methyl)amino]pyrimidine-2-carbonitrile (5.2 g, 24.947 mmol) was dissolved in anhydrous THE (104 mL) under nitrogen and the solution was cooled to −78° C. (acetone & dry-ice bath). After 10 min, diisobutylaluminum hydride in toluene (27.5 mL of 1 M, 27.500 mmol) was added dropwise (over 45 min) to the reaction. The reaction was then stirred at the same temperature for another 30 min (after addition was complete). Further Diisobutylaluminum hydride in toluene (5 mL of 1 M, 5.0000 mmol) was added over 15 min and stirred for another 40 min. The reaction was then quenched with Methanol (25 mL) at −78° C. and stirred for 15 min at the same temperature. After this time reaction mixture was warmed up to RT and 2 M HCl was added and pH was adjusted to pH=3. The organic layer was extracted EtOAc (3×300 mL) and combined organic solution was then washed with brine (200 mL). The organic solution was then dried over anhydrous sodium sulfate, filtered and solvent was removed by rotary evaporation to give crude mixture (weight=12.1 g). The crude product was then purified by flash chromatography (dry loaded in 330 g cartridge and eluting with 0-5% MeOH (3% NH₄OH) in DCM over 60 min and solvent was removed to provide 5-[2,2-dimethylpropyl(methyl)amino]pyrimidine-2-carbaldehyde (2.72 g, 51%) as light yellow. ESI-MS m/z calc. 207.13716, found 208.0 (M+1)⁺; Retention time: 1.73 minutes; LC method W. ¹H NMR(500 MHz, DMSO-d₆) δ 9.74 (s, 1H), 8.51 (s, 2H), 3.41 (s, 2H), 3.14 (s, 3H), 0.95 (s, 9H)

### Step 3: 3-[[4-(2,6-Dimethylphenyl)-6-[(2R)-2-[[5-[2,2-dimethylpropyl(methyl)amino]pyrimidin-2-yl]methylamino]-4,4-dimethyl-pentoxy]pyrimidin-2-yl]sulfamoyl]benzoic acid and 3-[[4-(2,6-dimethylphenyl)-6-[(2R)-2-[[5-(2,2-dimethylpropylamino)pyrimidin-2-yl]methylamino]-4,4-dimethyl-pentoxy]pyrimidin-2-yl]sulfamoyl]benzoic acid

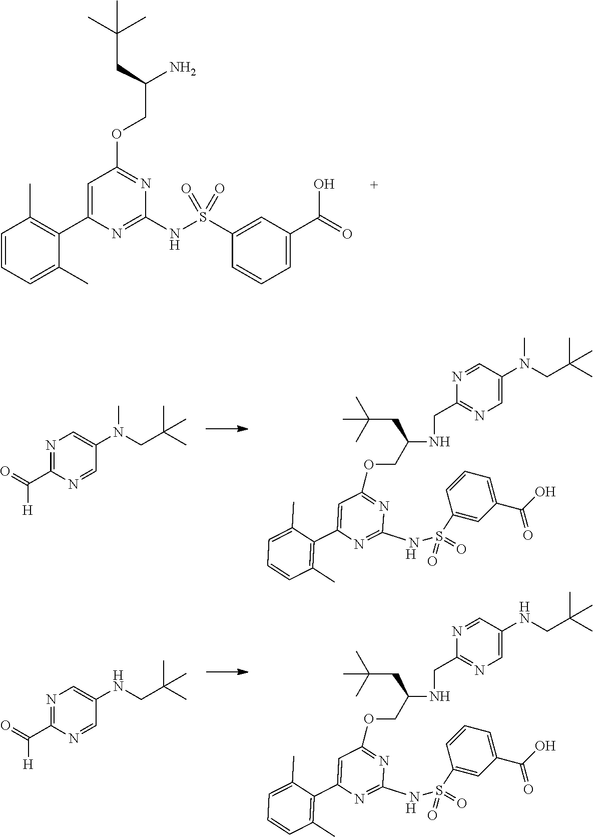

In a 4 mL vial, to a stirred heterogeneous mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (50 mg, 0.09106 mmol) in anhydrous dichloromethane (0.5 mL) were added 5-[2,2-dimethylpropyl(methyl)amino]pyrimidine-2-carbaldehyde (19 mg, 0.09167 mmol), and glacial acetic acid (10 μL, 0.1758 mmol), in that order. The vial was purged with nitrogen briefly and capped and stirred for 5 min, then sodium triacetoxyborohydride (100 mg, 0.4718 mmol) was added, followed by addition of DIEA (50 μL, 0.2871 mmol), and the capped vial was allowed to stir at ambient temperature for 1 h. Then methanol (0.3 mL) and water (0.2 mL) were added to the reaction and the volatiles were removed under reduced pressure and the residue was taken up in DMSO (1.5 mL), and purified by (reverse-phase HPLC, 1-99% acetonitrile in water over 15 min, HCl as modifier) to furnish 3-[[4-(2,6-dimethylphenyl)-6-[(2R)-2-[[5-[2,2-dimethylpropyl(methyl)amino]pyrimidin-2-yl]methylamino]-4,4-dimethyl-pentoxy]pyrimidin-2-yl]sulfamoyl]benzoic acid (Dihydrochloride salt) (18 mg, 25%) as a white solid. It was used in the subsequent reaction. ESI-MS m/z calc. 703.35156, found 704.4 (M+1)⁺; Retention time: 1.48 minutes; LC method A.

Following the above protocol, 5-(2,2-dimethylpropylamino)pyrimidine-2-carbaldehyde (18 mg, 0.09315 mmol) was employed, 3-[[4-(2,6-dimethylphenyl)-6-[(2R)-2-[[5-(2,2-dimethylpropylamino)pyrimidin-2-yl]methylamino]-4,4-dimethyl-pentoxy]pyrimidin-2-yl]sulfamoyl]benzoic acid (Dihydrochloride salt) (20 mg, 29%) was obtained as a white solid. It was used in the subsequent reaction. ESI-MS m/z calc. 689.33594, found 690.4 (M+1)⁺; Retention time: 1.54 minutes; LC method A.

### Step 4: (11R)-6-(2,6-Dimethylphenyl)-11-(2,2-dimethylpropyl)-12-[[5-[2,2-dimethylpropyl(methyl)amino]pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 115)

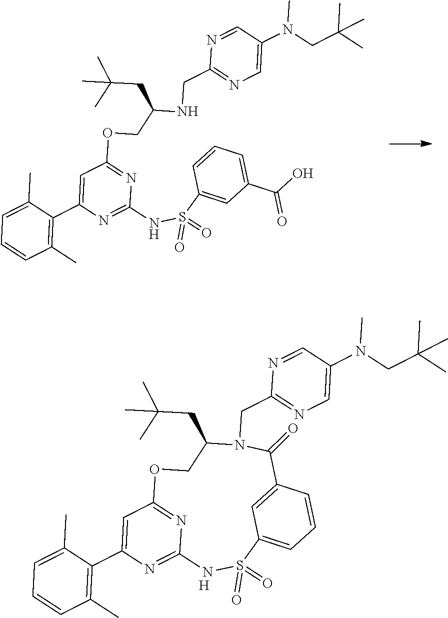

In a 4 mL vial, to a stirred solution of 3-[[4-(2,6-dimethylphenyl)-6-[(2R)-2-[[5-[2,2-dimethylpropyl(methyl)amino]pyrimidin-2-yl]methylamino]-4,4-dimethyl-pentoxy]pyrimidin-2-yl]sulfamoyl]benzoic acid (Dihydrochloride salt) (15 mg, 0.01931 mmol) in anhydrous DMF (1 mL) was added [dimethylamino(triazolo[4,5-b]pyridin-3-yloxy)methylene]-dimethyl-ammonium (Phosphorus Hexafluoride Ion) (15 mg, 0.03945 mmol) (HATU), followed by addition of DIEA (20 μL, 0.1148 mmol) at ambient temperature. The vial was briefly purged with nitrogen and the capped vial was allowed to stir at ambient temperature for 2 h. The orange solution was micro-filtered, and purified by preparative reverse-phase HPLC (1-99% acetonitrile in water over 15 min, 5 mM HCl as modifier), to give (11R)-6-(2,6-dimethylphenyl)-11-(2,2-dimethylpropyl)-12-[[5-[2,2-dimethylpropyl(methyl)amino]pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (7 mg, 50%) as a white solid. ESI-MS m/z calc. 685.341, found 686.5 (M+1)⁺; Retention time: 1.94 minutes; LC method A. ¹H NMR (400 MHz, DMSO-d₆) δ 13.03 (s, 1H), 8.68 (s, 1H), 8.32 (s, 2H), 7.95 (d, J 6.8 Hz, 1H), 7.66 (s, 2H), 7.26 (t, J 7.6 Hz, 1H), 7.13 (d, J 7.6 Hz, 2H), 6.41 (s, 1H), 5.37 (dd, J 10.7, 4.0 Hz, 1H), 4.82 (d, J 16.2 Hz, 1H), 4.54 (d, J 16.3 Hz, 1H), 4.13 (t, J 11.1 Hz, 1H), 4.08-3.97 (m, 1H), 3.23 (d, J 4.7 Hz, 2H), 3.00 (s, 3H), 2.27-1.84 (m, 6H), 1.78 (dd, J 15.2, 8.8 Hz, 1H), 1.38 (d, J 15.0 Hz, 1H), 0.94 (s, 9H), 0.56 (s, 9H).

### Example 91: Preparation of Compound 116

### Step 1: (11R)-6-(2,6-Dimethylphenyl)-11-(2,2-dimethylpropyl)-12-[[5-(2,2-dimethylpropylamino)pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 116)

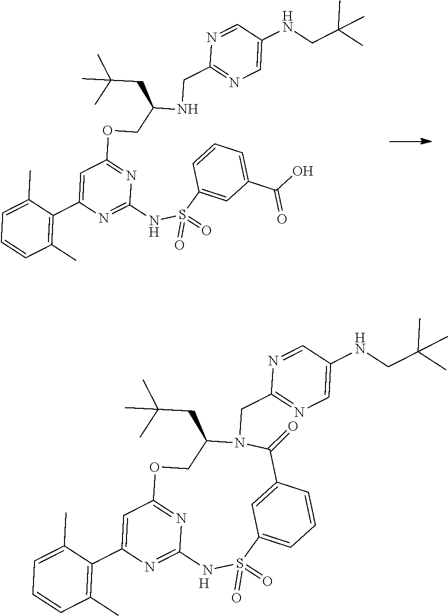

In a 4 mL vial, to a stirred solution of 3-[[4-(2,6-dimethylphenyl)-6-[(2R)-2-[[5-(2,2-dimethylpropylamino)pyrimidin-2-yl]methylamino]-4,4-dimethyl-pentoxy]pyrimidin-2-yl]sulfamoyl]benzoic acid (Dihydrochloride salt) (18 mg, 0.02360 mmol) in anhydrous DMF (1 mL) was added [dimethylamino(triazolo[4,5-b]pyridin-3-yloxy)methylene]-dimethyl-ammonium (Phosphorus Hexafluoride Ion) (18 mg, 0.04734 mmol) (HATU), followed by addition of DIEA (25 μL, 0.1435 mmol) at ambient temperature. The vial was briefly purged with nitrogen and the capped vial was allowed to stir at ambient temperature for 2 h. The orange solution was micro-filtered, and purified by preparative reverse-phase HPLC (1-99% acetonitrile in water over 15 min, 5 mM HCl as modifier), to give (11R)-6-(2,6-dimethylphenyl)-11-(2,2-dimethylpropyl)-12-[[5-(2,2-dimethylpropylamino)pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (2.5 mg, 15%) as a white solid. ESI-MS m/z calc. 671.3254, found 672.5 (M+1)⁺; Retention time: 1.87 minutes; LC method A.

### Example 92: Preparation of Compound 117

### Step 1: 5-(4,4-Dimethyl-1-piperidyl)pyrimidine-2-carbonitrile

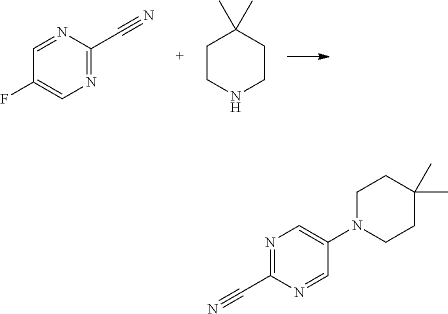

To a stirred solution of 5-fluoropyrimidine-2-carbonitrile (3.00 g, 24.37 mmol) in anhydrous dimethyl sulfoxide (50 mL) was added 4,4-dimethylpiperidine (hydrochloride salt)(4.12 g, 27.53 mmol), followed by addition of potassium carbonate (9.00 g, 65.12 mmol). The heterogenous mixture was purged with nitrogen briefly and stirred at 60° C. under nitrogen for 4 h. After allowing the tan reaction mixture to cool to ambient temperature. It was poured over ice-water (200 mL) and the slurry was stirred for 10 min, then filtered and the filter cake was washed with water (4×40 mL), and dried under vacuum to furnish 5-(4,4-dimethyl-1-piperidyl)pyrimidine-2-carbonitrile (5.12 g, 97%) as a tan light-weighed solid. ¹H NMR (400 MHz, DMSO-d₆) δ 8.55 (s, 2H), 3.53-3.42 (m, 4H), 1.47-1.35 (m, 4H), 0.97 (s, 6H). ESI-MS m/z calc. 216.1375, found 217.1 (M+1)⁺; Retention time: 1.48 minutes (LC method A).

### Step 2: 5-(4,4-Dimethyl-1-piperidyl)pyrimidine-2-carbaldehyde

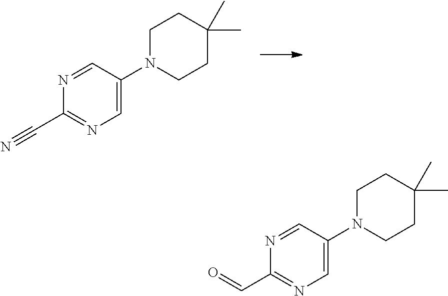 [SMILES: CC1(C)CCN(c2cnc(C#N)nc2)CC1 ; CC1(C)CCN(c2cnc(C=O)nc2)CC1]

Diisobutylaluminum hydride (in toluene) (10.2 mL of 1 M, 10.200 mmol) was added dropwise to 5-(4,4-dimethyl-1-piperidyl)pyrimidine-2-carbonitrile (2 g, 9.2472 mmol) dissolved in dry THE (80 mL) at −75° C. (internal temperature, slight exotherm) then stirred for 2 hours, then a freshly prepared saturated aqueous solution of Rochelle salts was added to the mixture (80 mL). The biphasic mixture was stirred overnight at room temperature. The aqueous phase was separated then extracted with ethyl acetate (3×50 ml), the organic phases were combined, dried with sodium sulfate, filtered and concentrated under reduced pressure. The remaining crude was purified by reverse phase chromatography (30 g) eluting with a gradient of MeCN in water (5% to 100%) to provide 5-(4,4-dimethyl-1-piperidyl)pyrimidine-2-carbaldehyde (1.303 g, 61%) as a yellow solid. ¹H NMR (400 MHz, CDCl₃) δ 9.92 (s, 1H), 8.45 (s, 2H), 3.48-3.43 (m, 4H), 1.58-1.52 (m, 4H), 1.04 (s, 6H).

### Step 3: 3-[[4-(2,6-dimethylphenyl)-6-[(2R)-2-[[5-(4,4-dimethyl-1-piperidyl)pyrimidin-2-yl]methylamino]-4,4-dimethyl-pentoxy]pyrimidin-2-yl]sulfamoyl]benzoic acid

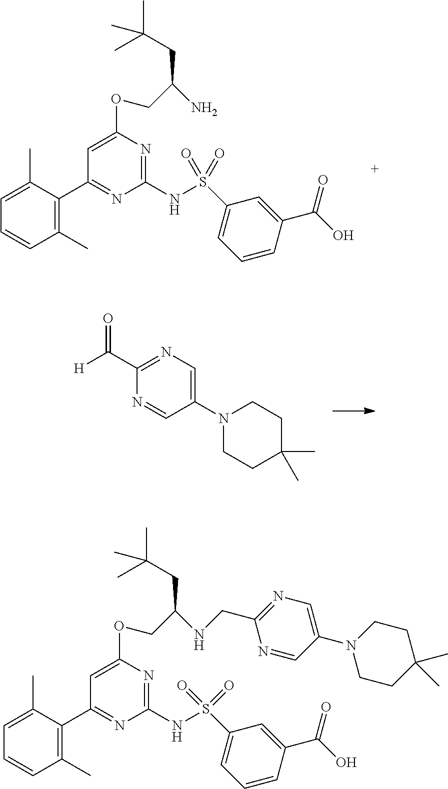 [SMILES: Cc1cccc(C)c1-c1cc(OC[C@H](N)CC(C)(C)C)nc(NS(=O)(=O)c2cccc(C(=O)O)c2)n1 ; CC1(C)CCN(c2cnc(C=O)nc2)CC1 ; Cc1cccc(C)c1-c1cc(OC[C@@H](CC(C)(C)C)NCc2ncc(N3CCC(C)(C)CC3)cn2)nc(NS(=O)(=O)c2cccc(C(=O)O)c2)n1]

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (40 mg, 0.0728 mmol) in dichloromethane (2 mL) was added a solution of 5-(4,4-dimethyl-1-piperidyl)pyrimidine-2-carbaldehyde (40 mg, 0.1204 mmol) in dichloromethane (2 mL) then acetic acid (21.120 mg, 0.02 mL, 0.3517 mmol) and the reaction was stirred at room temperature for 45 minutes. Sodium triacetoxyborohydride (107 mg, 0.4897 mmol) was added and the reaction mixture was stirred at room temperature for 1.5 hours. An aqueous 1N HCl solution (10 mL) was added and the phases were separated. The aqueous layer was extracted with DCM (3×10 mL). The combined organic layers were dried over sodium sulfate, filtered and concentrated under reduced pressure to afford the crude 3-[[4-(2,6-dimethylphenyl)-6-[(2R)-2-[[5-(4,4-dimethyl-1-piperidyl)pyrimidin-2-yl]methylamino]-4,4-dimethyl-pentoxy]pyrimidin-2-yl]sulfamoyl]benzoic acid (hydrochloride salt) (110 mg, 121%) as a yellow solid. ESI-MS m z calc. 715.35156, found 716.2 (M+1)⁺; Retention time: 1.65 minutes; LC method X

### Step 4: (11R)-6-(2,6-dimethylphenyl)-12-[[5-(4,4-dimethyl-1-piperidyl)pyrimidin-2-yl]methyl]-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 117)

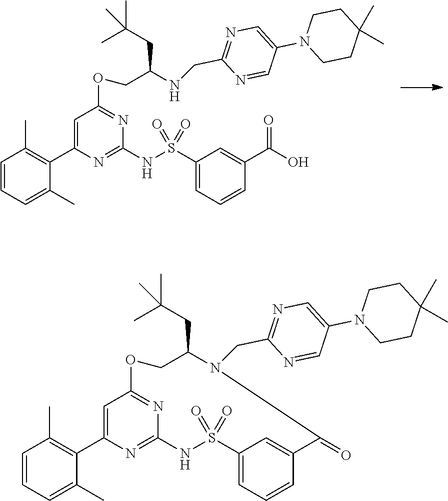 [SMILES: Cc1cccc(C)c1-c1cc(OC[C@@H](CC(C)(C)C)NCc2ncc(N3CCC(C)(C)CC3)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ncc(N3CCC(C)(C)CC3)cn1)[C@H](CC(C)(C)C)CO2]

To a stirred solution of N-methylmorpholine (653.20 mg, 0.71 mL, 6.4579 mmol) in dimethylformamide (DMF) (155 mL) at 0° C. was added 2-chloro-4,6-dimethoxy-1,3,5-triazine (353 mg, 2.0106 mmol) followed by 3-[[4-(2,6-dimethylphenyl)-6-[(2R)-2-[[5-(4,4-dimethyl-1-piperidyl)pyrimidin-2-yl]methylamino]-4,4-dimethyl-pentoxy]pyrimidin-2-yl]sulfamoyl]benzoic acid (hydrochloride salt) (748 mg, 0.9564 mmol) in dimethylformamide (DMF) (15 mL). The reaction mixture was stirred at 0° C. for 5 minutes. Then the reaction was warmed up to room temperature and stirred at room temperature for 72 hours. The reaction mixture was concentrated under reduced pressure at 50° C. The remaining crude was purified by flash chromatography (24 g), eluting with a gradient of EtOAc in Heptanes (10 to 100% in 21 CV). Removal of the volatiles under reduced pressure afforded a yellow solid which was purified by reverse phase chromatography on a 30 g C₁₈ cartridge using a gradient of MeCN in water (10% to 100%, 21CV then 100%, 3CV). The fractions containing the product were evaporated and then lyophilized to give (11R)-6-(2,6-dimethylphenyl)-12-[[5-(4,4-dimethyl-1-piperidyl)pyrimidin-2-yl]methyl]-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (246.6 mg, 37%) as an off-white solid. ESI-MS m/z calc. 697.341, found 698.3 (M+1)⁺; Retention time: 4.8 minutes; LC method Y. ¹H NMR (400 MHz, DMSO-d₆) δ 8.69 (br s., 1H), 8.48 (s, 2H), 7.99-7.91 (m, 1H), 7.74-7.61 (m, 2H), 7.30-7.22 (m, 1H), 7.16-7.08 (m, 2H), 6.42 (br s., 1H), 5.42-5.34 (m, 1H), 4.87-4.78 (m, 1H), 4.62-4.53 (m, 1H), 4.19-4.10 (m, 1H), 4.08-3.98 (m, 1H), 3.28-3.22 (m, 4H), 2.21-1.85 (m, 6H), 1.83-1.73 (m, 1H), 1.47-1.38 (m, 5H), 0.96 (s, 6H), 0.57 (s, 9H).

### Example 93: Preparation of Compound 118

### Step 1: 5-Hydroxypyrimidine-2-carbonitrile

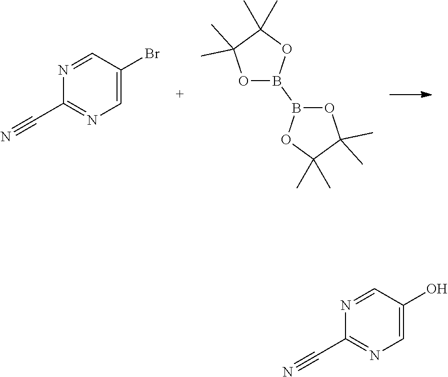

Into a reaction flask was charged with 5-bromopyrimidine-2-carbonitrile (10 g, 54.350 mmol), 4,4,5,5-tetramethyl-2-(4,4,5,5-tetramethyl-1,3,2-dioxaborolan-2-yl)-1,3,2-dioxaborolane (20.75 g, 81.713 mmol) and Potassium acetate (16.1 g, 164.05 mmol) in anhydrous 1,4-dioxane (100 mL). The reaction was purged with nitrogen for 10 minutes. Under nitrogen Pd(dppf)Cl₂ (2 g, 2.7333 mmol) was added. The reaction was purged with nitrogen for another 5 minutes. The reaction was then heated at 80° C. and stirred for 3 hours. The reaction was cooled to rt and diluted with 10% ammonium chloride aqueous solution (100 mL). The reaction was acidified to pH 1 with 3N aqueous HCl. The reaction was extracted with ethyl acetate (5×100 mL). The combined organic layers were washed with brine (100 mL), dried over anhydrous sodium sulfate and concentrated under vacuum. The crude boronic ester was dissolved in THE (100 mL) and water (100 mL). sodium perborate tetrahydrate (47.6 g, 309.37 mmol) was added to the reaction mixture. The reaction was stirred at rt overnight. The volatiles were removed under vacuum. The pH was adjusted to 10 with 15% aq. NaOH. The solid was removed by filtration. The filtrate was washed with ether (3×100 mL). Ether layers were discarded. The aqueous layer was acidified with concentrated HCl to pH 1 and was extracted with ethyl acetate (5×100 mL). The combined organic layers were washed with brine (100 mL), dried over anhydrous sodium sulfate and concentrated under vacuum. The residue was purified by silica gel chromatography using 0-5% MeOH in DCM to furnish the crude product (9.23 g). It was further purified by HPLC (Buffer A: water buffered with 5 mM HCl; Buffer B: ACN, eluting from 10 to 50% in 40 min). The fractions containing the product were combined and rotary evaporated to remove ACN. The pH was then adjusted to 4 using conc. HCl and the aqueous layer was extracted with 3:1 chloroform/isopropanol (3×100 mL). The combined organic solutions were dried over anhydrous sodium sulfate, filtered, and rotary evaporated. The residue was further dried in vacuo overnight yielding 5-hydroxypyrimidine-2-carbonitrile (2.7 g, 41%) as a white solid. ¹H NMR (500 MHz, DMSO) δ 11.81 (s, 1H), 8.45 (s, 2H).ESI-MS m/z calc. 121.02761, no ionization, Retention time: 1.43 minutes; LC method T.

### Step 2: 5-Isopropoxypyrimidine-2-carbonitrile

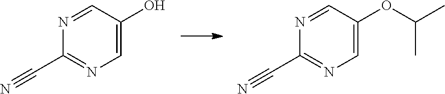

A solution of propan-2-ol (3.5325 g, 4.5 mL, 58.782 mmol), triphenylphosphine (5.8 g, 22.113 mmol), and 5-hydroxypyrimidine-2-carbonitrile (3.12 g, 15.459 mmol) in DCM (20 mL) was added with a solution of Bis(4-chlorobenzyl) azodicarboxylate (DCAD, 7.55 g, 20.562 mmol) in DCM (60 mL) dropwise at rt. The resulting orange solution was stirred at rt overnight. The reaction mixture was filtered, and the filtrate was concentrated. The residue was dry loaded onto silica and purified by flash chromatography (220 g column, 0 to 20% EtOAc in hexanes) yielding 5-isopropoxypyrimidine-2-carbonitrile (2.59 g, 100%) as a white solid. ¹H NMR (500 MHz, DMSO) δ 8.72 (s, 2H), 4.96 (hept, J 6.0 Hz, 1H), 1.33 (d, J 6.0 Hz, 6H). ESI-MS m/z calc. 163.07455, Retention time: 1.79 minutes; LC method W.

### Step 3: 5-Isopropoxypyrimidine-2-carbaldehyde

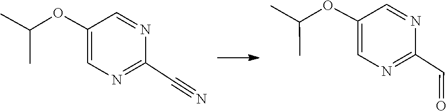

5-Isopropoxypyrimidine-2-carbonitrile (0.5 g, 2.9722 mmol) was dissolved in anhydrous THE (14 mL) under nitrogen and was cooled to −78° C. using an acetone-dry ice bath (stirred at this temperature for 30 min). DIBAL in toluene (3.6 mL of 1 M, 3.6000 mmol) was added dropwise over 15 min to the reaction mixture. The reaction was then stirred at the same temperature for 1 h. LCMS showed some SM left so more DIBAL in toluene (0.6 mL of 1 M, 0.6000 mmol) was added and stirring was continued at the same temperature for 1 h. The reaction was then quenched with saturated aq. sodium potassium tartrate solution (10 mL) and stirred for 15 min. More Rochelle's salt solution (100 mL) and EtOAc (100 mL) were added and the reaction mixture was stirred and allowed to warm up to RT over 2 h. Layers were separated, and the aqueous layer was further extracted with EtOAc (2×100 ml). The combined organic solutions were then washed with brine (150 ml), dried over anhydrous sodium sulfate, filtered, and concentrated. The residue was dried in vacuo for 30 min. The crude 5-isopropoxypyrimidine-2-carbaldehyde (0.56 g, 100%) (brown oil) was used in the next step without further purification. ESI-MS m/z calc. 166.07423, found 166.9 (M+1)⁺; Retention time: 1.77 minutes; LC method T.

### Step 4: 3-[[4-(2,6-Dimethylphenyl)-6-[(2R)-2-[(5-isopropoxypyrimidin-2-yl)methylamino]-4,4-dimethyl-pentoxy]pyrimidin-2-yl]sulfamoyl]benzoic acid

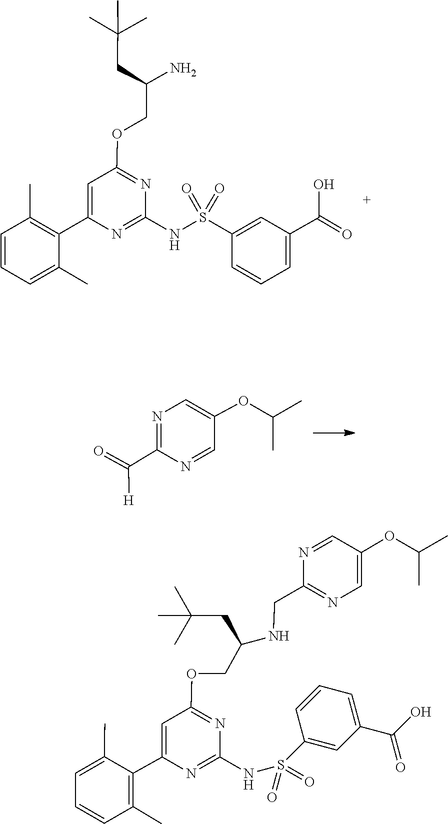 [SMILES: Cc1cccc(C)c1-c1cc(OC[C@H](N)CC(C)(C)C)nc(NS(=O)(=O)c2cccc(C(=O)O)c2)n1 ; CC(C)Oc1cnc(C=O)nc1 ; Cc1cccc(C)c1-c1cc(OC[C@@H](CC(C)(C)C)NCc2ncc(OC(C)C)cn2)nc(NS(=O)(=O)c2cccc(C(=O)O)c2)n1]

The compound was prepared in a manner analogous to that described above, using 5-isopropoxypyrimidine-2-carbaldehyde (17 mg, 0.1023 mmol) to give 3-[[4-(2,6-dimethylphenyl)-6-[(2R)-2-[(5-isopropoxypyrimidin-2-yl)methylamino]-4,4-dimethyl-pentoxy]pyrimidin-2-yl]sulfamoyl]benzoic acid (Hydrochloride salt) (2 mg, 3%), obtained as a white solid. ESI-MS m/z calc. 662.28864, found 663.4 (M+1)⁺; Retention time: 1.3 minutes; (LC method A).

### Step 5: (11R)-6-(2,6-Dimethylphenyl)-11-(2,2-dimethylpropyl)-12-{[5-(propan-2-yloxy)pyrimidin-2-yl]methyl}-9-oxa-2λ⁶-thia-3,5,12,19-tetraazatricyclo[12.3.1.14,8]nonadeca-1(17),4,6,8(19),14(18),15-hexaene-2,2,13-trione (Compound 118)

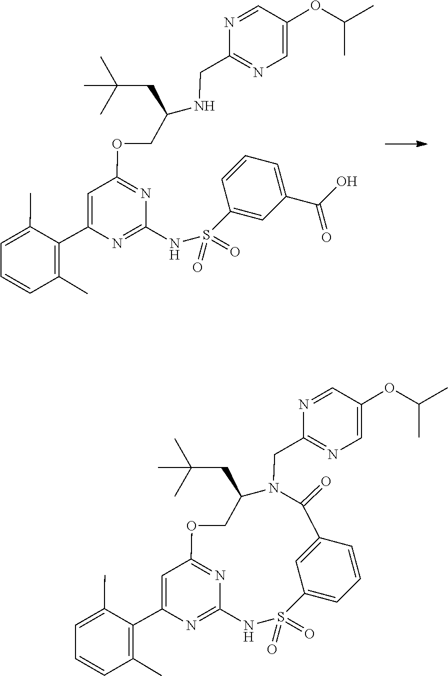 [SMILES: Cc1cccc(C)c1-c1cc(OC[C@@H](CC(C)(C)C)NCc2ncc(OC(C)C)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ncc(OC(C)C)cn1)[C@H](CC(C)(C)C)CO2]

To a stirred solution of 3-[[4-(2,6-dimethylphenyl)-6-[(2R)-2-[(5-isopropoxypyrimidin-2-yl)methylamino]-4,4-dimethyl-pentoxy]pyrimidin-2-yl]sulfamoyl]benzoic acid (Hydrochloride salt) (2 mg, 0.002860 mmol) in anhydrous DMF (0.2 mL) was added 2-chloro-4,6-dimethoxy-1,3,5-triazine (2 mg, 0.01139 mmol) (CDMT), followed by addition of 4-methylmorpholine (5 μL, 0.04548 mmol) at 0-4° C. (ice-water bath) under nitrogen. The clear reaction was allowed to stir at that temperature for 15 min, then allowed to stir at room temperature for 13 h (overnight). The product was purified by preparative reverse-phase HPLC (1-99% acetonitrile in water over 15 min, HCl as modifier) to furnish (11R)-6-(2,6-dimethylphenyl)-11-(2,2-dimethylpropyl)-12-{[5-(propan-2-yloxy)pyrimidin-2-yl]methyl}-9-oxa-2λ⁶-thia-3,5,12,19-tetraazatricyclo[12.3.1.14,8]nonadeca-1(17),4,6,8(19),14(18),15-hexaene-2,2,13-trione (1.1 mg, 59%) as a white solid. ESI-MS m/z calc. 644.2781, found 645.4 (M+1)⁺; Retention time: 1.83 minutes (LC method A).

### Example 94: Preparation of Compound 118

### Step 1: 3-[[4-Chloro-6-(2,6-dimethylphenyl)pyrimidin-2-yl]-(methoxymethyl)sulfamoyl]benzoic acid

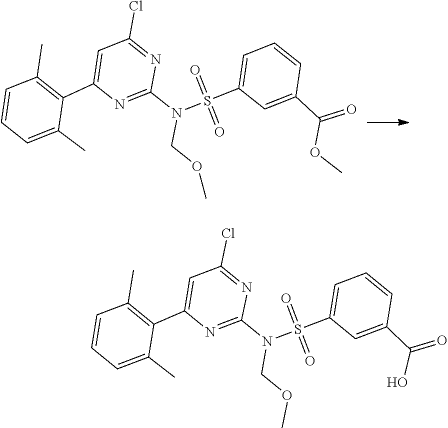

A mixture of methyl 3-[[4-chloro-6-(2,6-dimethylphenyl)pyrimidin-2-yl]-(methoxymethyl)sulfamoyl]benzoate (99 g, 208.01 mmol) in THE (1 L) and water (1 L) was treated with lithium hydroxide mono hydrate (13 g, 309.79 mmol) and stirred at room temperature for 4 h. Most of the THF was removed under reduced pressure, and the remaining aqueous layer was acidified to a pH of about 2-3 using 1N aqueous HCl (250 ml). The product was extracted with ethyl acetate (3×450 mL). The combined organic layers were washed with brine (100 mL), dried over sodium sulfate, filtered and concentrated under reduced pressure. The resulting sticky solid was triturated twice in EtOAc (150 ml and 100 ml) to afford 3-[[4-chloro-6-(2,6-dimethylphenyl)pyrimidin-2-yl]-(methoxymethyl)sulfamoyl]benzoic acid (55.1 g, 51%) as an off-white solid. ¹H NMR (400 MHz, DMSO-d₆) δ 13.37 (br. s., 1H), 8.47 (s, 1H), 8.21-8.09 (m, 2H), 7.61 (t, J 7.8 Hz, 1H), 7.44 (s, 1H), 7.30-7.20 (m, 1H), 7.11 (s, 1H), 7.09 (s, 1H), 5.61 (s, 2H), 3.30 (s, 3H), 1.84 (s, 6H). ESI-MS m/z calc. 461.0812, found 462.0 (M+1)⁺; Retention time: 4.32 minutes; LC method Y.

### Step 2: 3-[[4-[(2R)-2-Amino-4,4-dimethyl-pentoxy]-6-(2,6-dimethylphenyl)pyrimidin-2-yl]-(methoxymethyl)sulfamoyl]benzoic acid

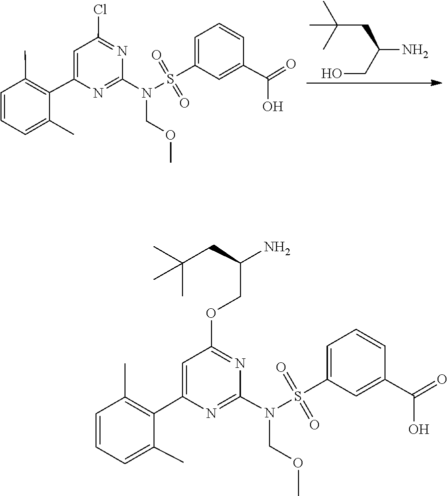

To a suspension of sodium hydride (4 g, 100.01 mmol) in THE (60 mL) was added (2R)-2-amino-4,4-dimethyl-pentan-1-ol (hydrochloride salt) (5.7 g, 33.994 mmol) and the reaction mixture was stirred at room temperature for 2 h. 3-[[4-Chloro-6-(2,6-dimethylphenyl)pyrimidin-2-yl]-(methoxymethyl)sulfamoyl]benzoic acid (16.2 g, 33.773 mmol) was added and the reaction was stirred at room temperature for 16 h. Aqueous 1N HCl was added until pH=2-3 was reached and the product was extracted with MeTHF (4×50 ml). Combined organic layers were washed with brine (40 mL), dried over sodium sulfate, filtered and concentrated under reduced pressure. The resulting crude was triturated from EtOAc (180 mL). Afforded 3-[[4-[(2R)-2-amino-4,4-dimethyl-pentoxy]-6-(2,6-dimethylphenyl)pyrimidin-2-yl]-(methoxymethyl)sulfamoyl]benzoic acid (hydrochloride salt) (14.8 g, 73%) as a white solid. ESI-MS m/z calc. 556.23553, found 557.2 (M+1)⁺; Retention time: 1.48 minutes; LC method X.

### Step 3: 5-Benzyloxy-2-(chloromethyl)pyrimidine

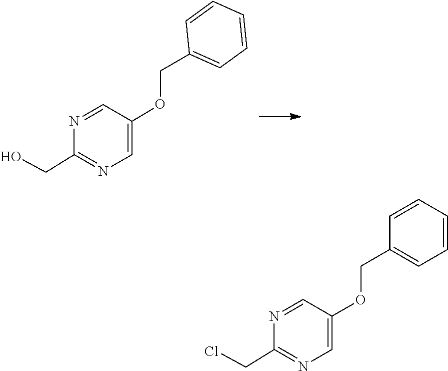 [SMILES: OCc1ncc(OCc2ccccc2)cn1 ; ClCc1ncc(OCc2ccccc2)cn1]

(5-Benzyloxypyrimidin-2-yl)methanol (2.5 g, 11.561 mmol) was dissolved in chloroform (100 mL). Nitrogen was bubbled for ten minutes after which time thionyl chloride (4.8930 g, 3 mL, 41.128 mmol) was added. The reaction was stirred at room temperature for 3 h. The reaction was concentrated under reduced pressure and the white residue was evaporated with chloroform (5×10 mL) then dried under high vacuum to afford 5-benzyloxy-2-(chloromethyl)pyrimidine (hydrochloride salt) (3.07 g, 98%) as an off-white solid. ¹H NMR (400 MHz, DMSO-d₆) δ 8.60 (s, 2H), 7.61-7.21 (m, 5H), 6.93-6.51 (m, 1H). 5.30 (s, 2H), 4.76 (s, 2H), ESI-MS m/z calc. 234.05598, found 235.2 (M+1)⁺; Retention time: 1.74 minutes; LC method X.

### Step 4: (11R)-6-(2,6-Dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

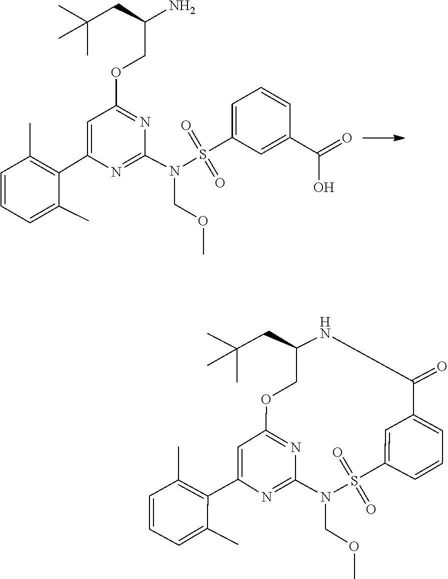 [SMILES: COCN(c1nc(OC[C@H](N)CC(C)(C)C)cc(-c2c(C)cccc2C)n1)S(=O)(=O)c1cccc(C(=O)O)c1 ; COCN1c2nc(cc(-c3c(C)cccc3C)n2)OC[C@@H](CC(C)(C)C)NC(=O)c2cccc(c2)S1(=O)=O]

To a solution of HATU (11.8 g, 31.034 mmol) and DIPEA (12.614 g, 17 mL, 97.599 mmol) in DMF (120 mL) was added a solution of 3-[[4-[(2R)-2-amino-4,4-dimethyl-pentoxy]-6-(2,6-dimethylphenyl)pyrimidin-2-yl]-(methoxymethyl)sulfamoyl]benzoic acid (hydrochloride salt) (14.8 g, 24.753 mmol) in DMF (110 mL). The reaction was stirred at room temperature for 20 min and poured into 370 ml of water. The resulting solid was filtered. The crude material was suspended in DMF (60 ml) and MeCN (50 mL) was added. The mixture was sonicated for 30 min and filtered. The solid was dissolved in DCM (100 mL) and the resulting organic layer was washed first with water (20 ml), then with a 1:1 v/v mix of water and brine (3×50 mL) and brine (2×20 mL). Afforded (11R)-6-(2,6-dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (5.3 g, 39%) as an off-white solid. ¹H NMR (400 MHz, DMSO-d₆) δ 8.72 (s, 1H), 8.14 (d, J 7.1 Hz, 1H), 7.97 (d, J 9.8 Hz, 1H), 7.88-7.76 (m, 2H), 7.22 (t, J 8.1 Hz, 1H), 7.12 (s, 1H), 7.10 (s, 1H), 6.72 (s, 1H), 5.70 (d, J 11.2 Hz, 1H), 5.53 (d, J 11.2 Hz, 1H), 5.04 (dd, J 11.2, 4.2 Hz, 1H), 3.87 (t, J 11.4 Hz, 1H), 3.57-3.45 (m, 1H), 2.88 (s, 3H), 1.96 (br. s., 6H), 1.57-1.41 (m, 2H), 0.51 (s, 9H). ESI-MS m/z calc. 538.225, found 539.2 (M+1)⁺; Retention time: 4.71 minutes; LC method Y.

### Step 5: (11R)-12-[(5-Benzyloxypyrimidin-2-yl)methyl]-6-(2,6-dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

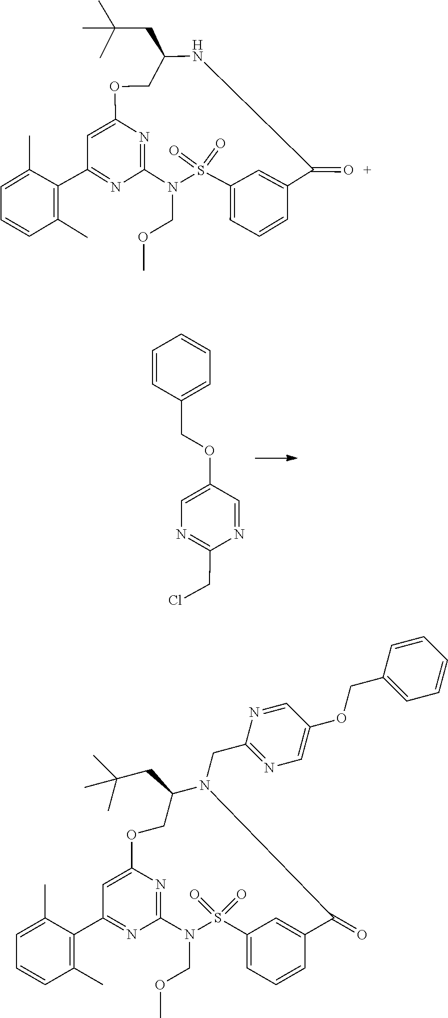 [SMILES: COCN1c2nc(cc(-c3c(C)cccc3C)n2)OC[C@@H](CC(C)(C)C)NC(=O)c2cccc(c2)S1(=O)=O ; ClCc1ncc(OCc2ccccc2)cn1 ; COCN1c2nc(cc(-c3c(C)cccc3C)n2)OC[C@@H](CC(C)(C)C)N(Cc2ncc(OCc3ccccc3)cn2)C(=O)c2cccc(c2)S1(=O)=O]

To a stirred solution of (11R)-6-(2,6-dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.02 g, 1.8936 mmol) in dry tetrahydrofuran (45 mL) were successively added 5-benzyloxy-2-(chloromethyl)pyrimidine (490 mg, 2.0879 mmol), tetrabutylammonium iodide (150 mg, 0.4061 mmol) and cesium carbonate (2.5 g, 7.6730 mmol) Nitrogen was bubbled and the reaction was stirred overnight at 50° C. Additional cesium carbonate (1.25 g, 3.8365 mmol) was added and the reaction was stirred at 50° C. for 24 h. A saturated aqueous solution of saturated ammonium chloride (200 mL) was added and the aqueous layer was extracted with ethyl acetate (5×75 mL). The combined organic extracts were washed with brine (250 mL) and water (250 mL), dried over sodium sulfate, filtered and concentrated under reduced pressure to afford a yellow foam which was purified by reverse phase chromatography on C₁₈ using a 120 g column and eluting with a gradient of acetonitrile in acidic water containing 0.1% weight of formic acid (5 to 100% in 10 CV then 100% for 3 CV). The desired fractions were concentrated under reduced pressure and residual water was evaporated with acetonitrile (3×10 mL) and freeze-dried to afford (11R)-12-[(5-benzyloxypyrimidin-2-yl)methyl]-6-(2,6-dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.04 g, 73%) as a white powder. ¹H NMR (400 MHz, DMSO-d₆) δ 8.85 (br. s, 1H), 8.61 (s, 2H), 8.19-8.12 (m, 1H), 7.85-7.77 (m, 2H), 7.51-7.45 (m, 2H), 7.44-7.38 (m, 2H), 7.38-7.32 (m, 1H), 7.26-7.19 (m, 1H), 7.11 (br. d, J 7.6 Hz, 2H), 6.72 (s, 1H), 5.74 (d, J 10.8 Hz, 1H), 5.57 (d, J 11.0 Hz, 1H), 5.33-5.24 (m, 3H), 4.88 (d, J 16.6 Hz, 1H), 4.70 (d, J 16.6 Hz, 1H), 4.28-4.09 (m, 2H), 2.97 (s, 3H), 2.05-1.87 (m, 6H), 1.78 (dd, J 15.0, 8.2 Hz, 1H), 1.44 (d, J 14.4 Hz, 1H), 0.53 (s, 9H). ESI-MS m/z calc. 736.3043, found 369.0 (M-367)⁺; Retention time: 2.46 minutes (LC method K).

### Step 6: (11R)-6-(2,6-Dimethylphenyl)-11-(2,2-dimethylpropyl)-12-[(5-hydroxypyrimidin-2-yl)methyl]-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

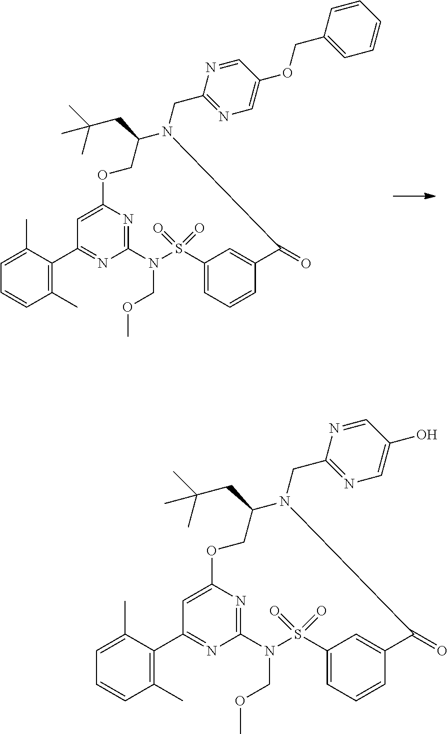 [SMILES: COCN1c2nc(cc(-c3c(C)cccc3C)n2)OC[C@@H](CC(C)(C)C)N(Cc2ncc(OCc3ccccc3)cn2)C(=O)c2cccc(c2)S1(=O)=O ; COCN1c2nc(cc(-c3c(C)cccc3C)n2)OC[C@@H](CC(C)(C)C)N(Cc2ncc(O)cn2)C(=O)c2cccc(c2)S1(=O)=O]

Palladium 5% on carbon (350 mg, 0.1644 mmol) was added into a 250 mL-flask and it was purged with nitrogen for 2 minutes. A solution of (11R)-12-[(5-benzyloxypyrimidin-2-yl)methyl]-6-(2,6-dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.04 g, 1.4114 mmol) in MeOH (100 mL) was then added into the flask. Hydrogen was bubbled into the suspension for 2 minutes and then the reaction mixture was stirred under 1 atm. of hydrogen for 30 minutes. Nitrogen was then bubbled into the mixture for 10 minutes. The reaction mixture was filtered on Celite, the pad was rinsed with methanol (50 mL) and the filtrate was concentrated in vacuo. The crude was purified by reverse phase chromatography (80 g C₁₈ cartridge) eluting with a gradient of MeCN in acidic water (0.1% v/v of formic acid in water) (5% for 5 CV then 50 to 100% for 20 CV). Afforded after evaporation and lyophilization (11R)-6-(2,6-dimethylphenyl)-11-(2,2-dimethylpropyl)-12-[(5-hydroxypyrimidin-2-yl)methyl]-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (731.8 mg, 80%) as a white solid. ESI-MS m/z calc. 646.2573, found 647.0 (M+1)⁺; Retention time: 3.39 minutes; LC method U.

### Step 7: (11R)-6-(2,6-Dimethylphenyl)-11-(2,2-dimethylpropyl)-12-[(5-isopropoxypyrimidin-2-yl)methyl]-3-(methoxymethyl)-2,2-dioxo-9-oxa-2×⁶-thia-3,5,12,19-tetrazatricyclo[12.3.1.14,8]nonadeca-1(18),4(19),5,7,14,16-hexaen-13-one

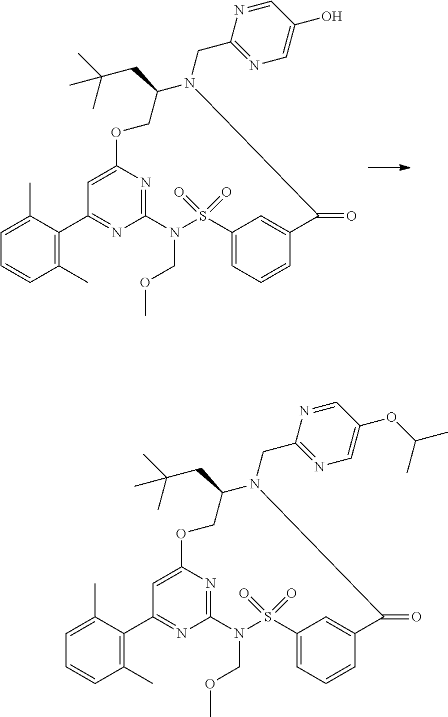 [SMILES: COCN1c2nc(cc(-c3c(C)cccc3C)n2)OC[C@@H](CC(C)(C)C)N(Cc2ncc(O)cn2)C(=O)c2cccc(c2)S1(=O)=O ; COCN1c2nc(cc(-c3c(C)cccc3C)n2)OC[C@@H](CC(C)(C)C)N(Cc2ncc(OC(C)C)cn2)C(=O)c2cccc(c2)S1(=O)=O]

To a solution of (11R)-6-(2,6-dimethylphenyl)-11-(2,2-dimethylpropyl)-12-[(5-hydroxypyrimidin-2-yl)methyl]-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (44 mg, 0.0680 mmol) in THE (0.7 mL) was added 2,2-dimethylpropan-1-ol (18 mg, 0.2042 mmol) and triphenylphosphine (55 mg, 0.2097 mmol) at 0° C. Diisopropyl azodicarboxylate (42.230 mg, 41 L, 0.2088 mmol) was then added and the mixture was stirred at 0° C. for 10 minutes and then at 40° C. for 3 hours. The crude was evaporated in vacuo and the mixture was purified on a 30 g Cis cartridge, eluting with a gradient of MeCN in acidic water (0.1% v/v of formic acid in water) (5% for 5 CV then 50 to 100% for 20 CV). Afforded after evaporation and lyophilization (11R)-6-(2,6-dimethylphenyl)-11-(2,2-dimethylpropyl)-12-[(5-isopropoxypyrimidin-2-yl)methyl]-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (44.8 mg, 95%) as a white solid. ¹H NMR (400 MHz, DMSO-d₆) δ 8.85 (s, 1H), 8.52 (s, 2H), 8.19-8.12 (m, 1H), 7.86-7.77 (m, 2H), 7.26-7.19 (m, 1H), 7.11 (d, J 7.8 Hz, 2H), 6.72 (s, 1H), 5.74 (d, J 11.0 Hz, 1H), 5.56 (d, J 11.0 Hz, 1H), 5.28 (dd, J 10.5, 3.7 Hz, 1H), 4.87 (d, J 16.6 Hz, 1H), 4.80 (dt, J 12.0, 6.0 Hz, 1H), 4.69 (d, J 16.6 Hz, 1H), 4.28-4.18 (m, 1H), 4.18-4.10 (m, 1H), 2.97 (s, 3H), 1.96 (br. s., 6H), 1.78 (dd, J 14.9, 8.6 Hz, 1H), 1.44 (d, J 14.9 Hz, 1H), 1.30 (d, J 2.0 Hz, 3H), 1.29 (d, J 2.0 Hz, 3H), 0.53 (s, 9H). ESI-MS m/z calc. 688.3043, found 689.3 (M+1)⁺; Retention time: 4.12 minutes; LC method 1D.

### Step 8: (11R)-6-(2,6-Dimethylphenyl)-11-(2,2-dimethylpropyl)-12-[(5-isopropoxypyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 118)

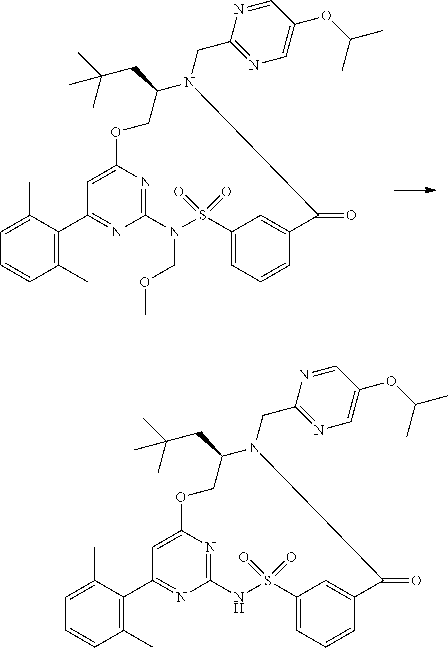 [SMILES: COCN1c2nc(cc(-c3c(C)cccc3C)n2)OC[C@@H](CC(C)(C)C)N(Cc2ncc(OC(C)C)cn2)C(=O)c2cccc(c2)S1(=O)=O ; Cc1cccc(C)c1-c1cc2nc(n1)NS(=O)(=O)c1cccc(c1)C(=O)N(Cc1ncc(OC(C)C)cn1)[C@H](CC(C)(C)C)CO2]

To a solution of (11R)-6-(2,6-dimethylphenyl)-11-(2,2-dimethylpropyl)-12-[(5-isopropoxypyrimidin-2-yl)methyl]-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (35 mg, 0.0504 mmol) in DCM (0.7 mL) was added HCl (in dioxane) (0.26 mL of 4 M, 1.0400 mmol). The mixture was stirred at room temperature for 3 hours and then the solvents were removed in vacuo. The crude was purified by reverse phase chromatography using a 15.5 g cartridge, eluting with a gradient of MeCN in water (containing 0.1% of formic acid) (5% for 5 CV then 50 to 100% in 20 CV). Afforded after evaporation and lyophilization (11R)-6-(2,6-dimethylphenyl)-11-(2,2-dimethylpropyl)-12-[(5-isopropoxypyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (23.7 mg, 73%) as a white solid. ¹H NMR (400 MHz, DMSO-d₆) δ 13.08 (br. s., 1H), 8.69 (br. s., 1H), 8.52 (s, 2H), 7.95 (br. s., 1H), 7.67 (br. s., 2H), 7.32-7.20 (m, 1H), 7.18-7.05 (m, 2H), 6.42 (br. s., 1H), 5.38 (d, J 7.8 Hz, 1H), 4.89-4.77 (m, 2H), 4.65 (d, J 16.6 Hz, 1H), 4.20 (t, J 11.1 Hz, 1H), 4.11-4.00 (m, 1H), 2.21-1.86 (m, 6H), 1.77 (dd, J 15.0, 9.2 Hz, 1H), 1.40 (d, J 14.7 Hz, 1H), 1.30 (d, J 2.2 Hz, 3H), 1.29 (d, J 2.4 Hz, 3H), 0.56 (s, 9H). ESI-MS m z calc. 644.2781, found 645.3 (M+1)⁺; Retention time: 3.09 minutes; LC method 1D.

### Example 95: Preparation of Compound 119

### Step 1: 5-(3,3-Dimethylpyrrolidin-1-yl)pyrimidine-2-carbonitrile

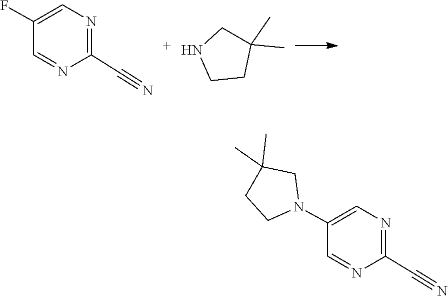

To a stirred solution of 5-fluoropyrimidine-2-carbonitrile (10 g, 77.181 mmol) and 3,3-dimethylpyrrolidine (hydrochloride salt) (13 g, 95.846 mmol) in DMF (100 mL) was added Cesium carbonate (63 g, 193.36 mmol) at rt and stirred for 6 h (The reaction was exothermic in the beginning). Water (400 mL) was added and the resulting precipitate was filtered. The cake was washed with water and dissolved in DCM (500 mL). The DCM layer was washed with brine, dried over sodium sulfate, filtered and concentrated in vacuo. The resulting beige solid was triturated with hexanes and filtered. The solid was washed with hexanes and dried in vacuo to give 5-(3,3-dimethylpyrrolidin-1-yl)pyrimidine-2-carbonitrile (15.1 g, 92%) as a white solid. ESI-MS m/z calc. 202.12184, found 203.5 (M+1)⁺; Retention time: 4.67 minutes; LC method S.

### Step 2: 5-(3,3-Dimethylpyrrolidin-1-yl)pyrimidine-2-carbaldehyde

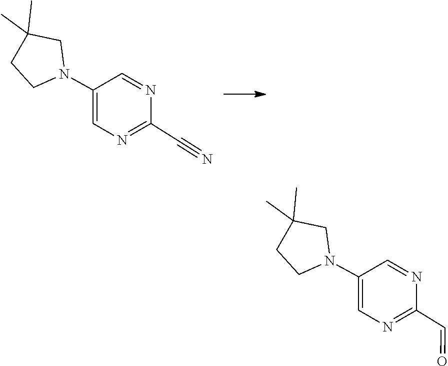

To a stirred solution of 5-(3,3-dimethylpyrrolidin-1-yl)pyrimidine-2-carbonitrile (14.7 g, 69.046 mmol) in THE (300 mL) was added DIBAL in toluene (85 mL of 1 M, 85.000 mmol) at −78° C. dropwise over 30 minutes and stirred for 2 h. The reaction mixture was quenched with 500 mL of saturated aqueous sodium potassium tartrate solution (Rochelle's salt) and stirred for 30 minutes while being allowed to warm to rt. The layers were separated, and the aqueous layer was extracted with EtOAc (3×300 mL). The combined organic layer was washed with brine, dried over sodium sulfate, filtered and concentrated in vacuo. The resulting red solid was purified by flash chromatography (silica gel, 220 g, loaded in DCM, eluted with 3% MeOH in DCM). The desired product fractions were combined and concentrated in vacuo to give 5-(3,3-dimethylpyrrolidin-1-yl)pyrimidine-2-carbaldehyde (8.08 g, 56%) as an orange solid. ¹H NMR (500 MHz, DMSO-d₆) δ 9.73 (s, 1H), 8.23 (s, 2H), 3.52 (t, J 7.0, 7.0 Hz, 2H), 3.21 (s, 2H), 1.80 (t, J 7.0, 7.0 Hz, 2H), 1.10 (s, 6H). ESI-MS m/z calc. 205.1215, found 206.2 (M+1)⁺; Retention time: 1.69 minutes; LC method W.

### Step 3: 3-[[4-(2,6-Dimethylphenyl)-6-[(2R)-2-[[5-(3,3-dimethylpyrrolidin-1-yl)pyrimidin-2-yl]methylamino]-4,4-dimethyl-pentoxy]pyrimidin-2-yl]sulfamoyl]benzoic acid

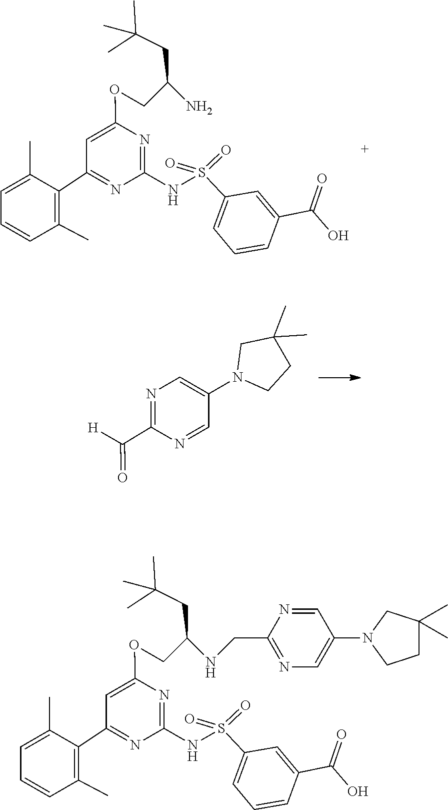 [SMILES: Cc1cccc(C)c1-c1cc(OC[C@H](N)CC(C)(C)C)nc(NS(=O)(=O)c2cccc(C(=O)O)c2)n1 ; CC1(C)CCN(c2cnc(C=O)nc2)C1 ; Cc1cccc(C)c1-c1cc(OC[C@@H](CC(C)(C)C)NCc2ncc(N3CCC(C)(C)C3)cn2)nc(NS(=O)(=O)c2cccc(C(=O)O)c2)n1]

To a 0° C. solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (200 mg, 0.3428 mmol) and 5-(3,3-dimethylpyrrolidin-1-yl)pyrimidine-2-carbaldehyde (75.2 mg, 0.3664 mmol) in anhydrous dichloromethane (20 mL) was added sodium triacetoxyborohydride (328 mg, 1.5476 mmol) and the reaction was stirred at room temperature for 40 min. The reaction was quenched at 0° C. by the addition of 15 mL of 1 N aqueous hydrochloric acid solution, brine (30 mL) and extracted with ethyl acetate (3×50 mL). The combined organics were concentrated under reduced pressure to provide 3-[[4-(2,6-dimethylphenyl)-6-[(2R)-2-[[5-(3,3-dimethylpyrrolidin-1-yl)pyrimidin-2-yl]methylamino]-4,4-dimethyl-pentoxy]pyrimidin-2-yl]sulfamoyl]benzoic acid (hydrochloride salt) (260 mg, 103%) as a light pink solid. ESI-MS m/z calc. 701.33594, found 702.2 (M+1)⁺; Retention time: 1.63 minutes; LC method X.

### Step 4: (11R)-6-(2,6-Dimethylphenyl)-11-(2,2-dimethylpropyl)-12-[[5-(3,3-dimethylpyrrolidin-1-yl)pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 119)

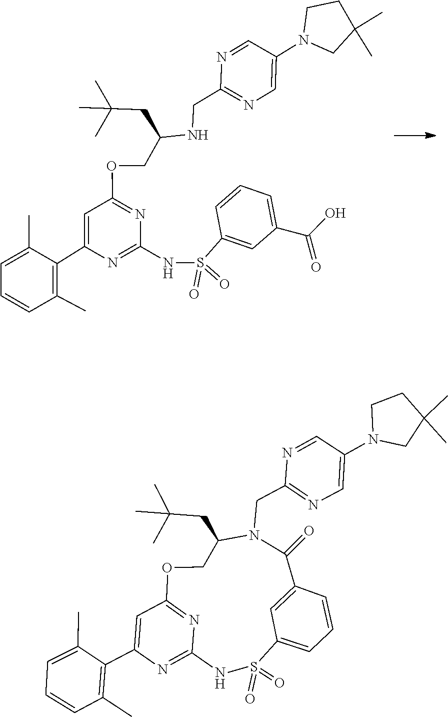 [SMILES: Cc1cccc(C)c1-c1cc(OC[C@@H](CC(C)(C)C)NCc2ncc(N3CCC(C)(C)C3)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ncc(N3CCC(C)(C)C3)cn1)[C@H](CC(C)(C)C)CO2]

To a stirred solution of 3-[[4-(2,6-dimethylphenyl)-6-[(2R)-2-[[5-(3,3-dimethylpyrrolidin-1-yl)pyrimidin-2-yl]methylamino]-4,4-dimethyl-pentoxy]pyrimidin-2-yl]sulfamoyl]benzoic acid (Dihydrochloride salt) (41 mg, 0.05292 mmol) in anhydrous DMF (1.8 mL) was added 2-chloro-4,6-dimethoxy-1,3,5-triazine (20 mg, 0.1139 mmol) (CDMT), followed by addition of 4-methylmorpholine (30 μL, 0.2729 mmol) at 0-4° C. (ice-water bath) under nitrogen. The clear reaction was allowed to stir at that temperature for 15 min, then allowed to stir at room temperature for 1 h. The product was purified by preparative reverse-phase HPLC (1-99% acetonitrile in water over 15 min, HCl as modifier) to furnish (11R)-6-(2,6-dimethylphenyl)-11-(2,2-dimethylpropyl)-12-[[5-(3,3-dimethylpyrrolidin-1-yl)pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (18 mg, 47%) as a white solid. ESI-MS m/z calc. 683.3254, found 684.5 (M+1)⁺; Retention time: 2.03 minutes; LC method A. ¹H NMR (400 MHz, DMSO-d₆) δ 12.92 (s, 1H), 8.69 (s, 1H), 8.19 (s, 2H), 7.95 (d, J 7.0 Hz, 1H), 7.76-7.53 (m, 2H), 7.26 (t, J 7.6 Hz, 1H), 7.12 (d, J 7.6 Hz, 2H), 6.40 (s, 1H), 5.38 (dd, J 10.5, 3.8 Hz, 1H), 4.86 (s, 1H), 4.54 (s, 1H), 4.20-3.92 (m, 2H), 3.24-3.16 (m, 2H), 3.08 (s, 2H), 2.00 (s, 6H), 1.86-1.73 (m, 3H), 1.40 (d, J 14.8 Hz, 1H), 1.10 (s, 6H), 0.57 (s, 9H).

### Example 96: Preparation of Compound 120

### Step 1: 3-[[4-[(2R)-2-[(5-Bromopyrimidin-2-yl)methylamino]-4,4-dimethyl-pentoxy]-6-(2,6-dimethylphenyl)pyrimidin-2-yl]sulfamoyl]benzoic acid

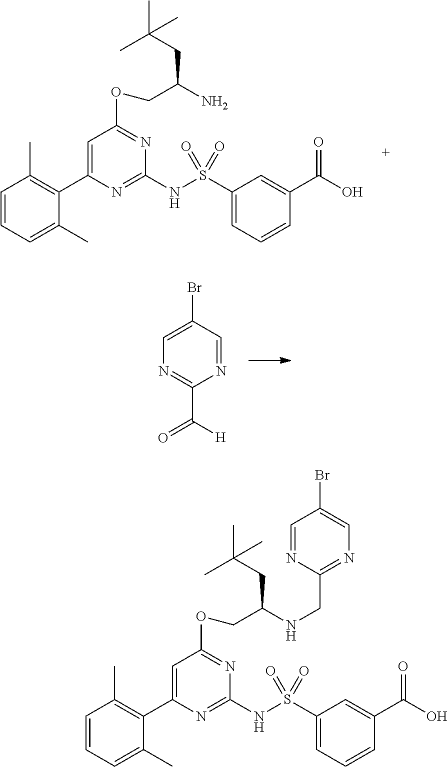 [SMILES: Cc1cccc(C)c1-c1cc(OC[C@H](N)CC(C)(C)C)nc(NS(=O)(=O)c2cccc(C(=O)O)c2)n1 ; O=Cc1ncc(Br)cn1 ; Cc1cccc(C)c1-c1cc(OC[C@@H](CC(C)(C)C)NCc2ncc(Br)cn2)nc(NS(=O)(=O)c2cccc(C(=O)O)c2)n1]

In a 25 mL flask, to a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (1.4 g, 2.4095 mmol) and 5-bromopyrimidine-2-carbaldehyde (1.6 g, 4.7916 mmol) in anhydrous dichloromethane (12 mL) were added acetic acid (glacial) (316.80 mg, 0.3 mL, 5.2754 mmol), triethylamine (617.10 mg, 0.85 mL, 6.0984 mmol) and sodium triacetoxyborohydride (2.5 g, 11.796 mmol), in that order. The vial was purged with nitrogen briefly and capped and allowed to stir at ambient temperature for 40 min under nitrogen. Then the mixture was poured over cold hydrochloric acid (20 mL of 1.0 M, 20.00 mmol) and brine (40 mL) and extracted with ethyl acetate (3×40 mL). The combined organics were concentrated under reduced pressure to provide 3-[[4-[(2R)-2-[(5-bromopyrimidin-2-yl)methylamino]-4,4-dimethyl-pentoxy]-6-(2,6-dimethylphenyl)pyrimidin-2-yl]sulfamoyl]benzoic acid (hydrochloride salt) (2.4 g, 89%) as a yellow solid. ESI-MS m/z calc. 682.1573, found 683.4 (M+1)⁺; Retention time: 1.49 minutes; LC method X.

### Step 2: (11R)-12-[(5-Bromopyrimidin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

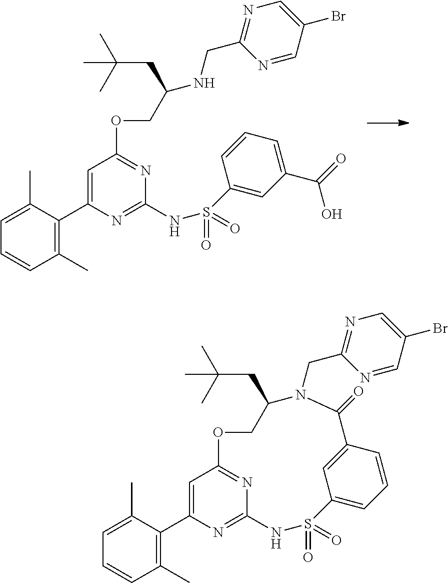 [SMILES: Cc1cccc(C)c1-c1cc(OC[C@@H](CC(C)(C)C)NCc2ncc(Br)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ncc(Br)cn1)[C@H](CC(C)(C)C)CO2]

To a 0° C. solution of N-methylmorpholine (395.60 mg, 0.43 mL, 3.9111 mmol) in DMF (55 mL) was added 2-chloro-4,6-dimethoxy-1,3,5-triazine (137 mg, 0.7803 mmol) followed by 3-[[4-[(2R)-2-[(5-bromopyrimidin-2-yl)methylamino]-4,4-dimethyl-pentoxy]-6-(2,6-dimethylphenyl)pyrimidin-2-yl]sulfamoyl]benzoic acid (hydrochloride salt) (352 mg, 0.4888 mmol). After 5 min the reaction was stirred at room temperature for 72 h. The reaction mixture was concentrated under reduced pressure at 50° C. The remaining crude was diluted with DCM (50 mL) and the solution was washed with a 1:1 v/v mix of water and brine (4×20 mL), water (30 mL) and brine (40 mL). The resulting organic layer was dried over sodium sulfate, filtered and concentrated under reduced pressure. The crude was purified by reverse phase chromatography on a 80 g C₁₈ cartridge using a gradient of MeCN in acidic water (0.1% v/v of formic acid in water) of 40 to 100% for 20 CV then 100% for 2 CV. After evaporation of MeCN, ethyl acetate (80 mL) and an aqueous solution of sodium bicarbonate (30 mL) were added. The aqueous phase was separated and extracted with ethyl acetate (2×40 mL). The combined organic layers were washed with brine (30 mL), dried over sodium sulfate, filtered and concentrated to afford (11R)-12-[(5-bromopyrimidin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (262 mg, 78%) as a white solid. ESI-MS m/z calc. 664.1467, found 665.2 (M+1)⁺; Retention time: 4.33 minutes; LC method Y. ¹H NMR (400 MHz, DMSO-d₆) δ 13.07 (br s, 1H), 9.06-8.97 (m, 2H), 8.73-8.62 (m, 1H), 8.01-7.88 (m, 1H), 7.76-7.56 (m, 2H), 7.34-7.20 (m, 1H), 7.18-7.04 (m, 2H), 6.49-6.34 (m, 1H), 5.42-5.31 (m, 1H), 4.87-4.80 (m, 1H), 4.79-4.72 (m, 1H), 4.33-4.20 (m, 1H), 4.10-3.98 (m, 1H), 2.14-1.87 (m, 6H), 1.82-1.72 (m, 1H), 1.46-1.36 (m, 1H), 0.56 (s, 9H).

### Step 3: (11R)-6-(2,6-Dimethylphenyl)-11-(2,2-dimethylpropyl)-12-[(5-isobutylpyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

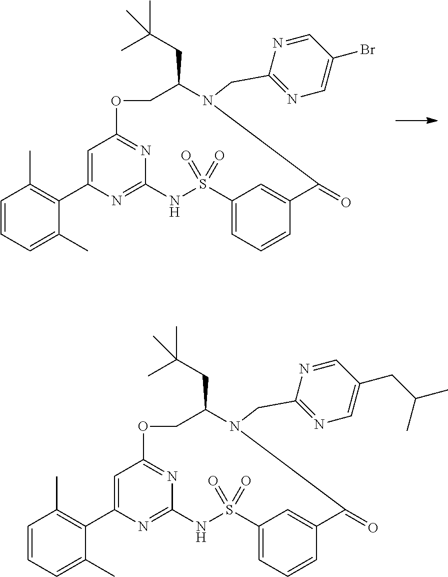 [SMILES: Cc1cccc(C)c1-c1cc2nc(n1)NS(=O)(=O)c1cccc(c1)C(=O)N(Cc1ncc(Br)cn1)[C@H](CC(C)(C)C)CO2 ; Cc1cccc(C)c1-c1cc2nc(n1)NS(=O)(=O)c1cccc(c1)C(=O)N(Cc1ncc(CC(C)C)cn1)[C@H](CC(C)(C)C)CO2]

In a sealed tube was introduced (11R)-12-[(5-bromopyrimidin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (26 mg, 0.0378 mmol), 4,4,5,5-tetramethyl-2-(2-methylprop-1-enyl)-1,3,2-dioxaborolane (30 mg, 0.1648 mmol), potassium carbonate (25 mg, 0.1809 mmol), and 1,1′-bis(diphenylphosphino)ferrocene palladium(II) chloride, complex with dichloromethane (6 mg, 0.0073 mmol). The tube was purged with nitrogen, then dioxane (1 mL) and water (0.1 mL) were added. The tube was capped, and the reaction mixture was stirred at temperature between 80 and 90° C. for 18 hours under a nitrogen atmosphere. The reaction mixture was cooled down to room temperature. ethyl acetate (10 mL) and water (1 mL) was added. The organics was separated, filtered (using a microfilter syringe) and concentrated under reduced pressure to provide crude (11R)-6-(2,6-dimethylphenyl)-11-(2,2-dimethylpropyl)-12-[[5-(2-methylprop-1-enyl)pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one ESI-MS m/z calc. 640.2832, found 641.2 (M+1)⁺; Retention time: 1.96 minutes, which was dissolved in methanol (1.6 mL) and then 10% Palladium on carbon 50% wet (50 mg, 0.0235 mmol) was added. Hydrogen was bubbled into the suspension for 90 min. LCMS showed the reaction was complete. The reaction mixture was filtered with a syringe filter. The filtrate was concentrated in vacuo. The crude product was purified by reverse phase chromatography on a 5.5 g C₁₈ cartridge using a 5 to 100% gradient of methanol in water (with 0.1% formic acid content). The fractions containing the product were concentrated under reduced pressure to afford (11R)-6-(2,6-dimethylphenyl)-11-(2,2-dimethylpropyl)-12-[(5-isobutylpyrimidin-2-yl)methyl]-2,2-dioxo-9-oxa-2λ⁶-thia-3, 5,12,19-tetrazatricyclo[12.3.1.14,8]nonadeca-1(18),4(19),5,7,14,16-hexaen-13-one (9.7 mg, 40%) as a beige fluffy solid after lyophilization. ESI-MS m/z calc. 642.2988, found 643.3 (M+1)⁺; Retention time: 4.58 minutes; LC method Y. ¹H NMR (400 MHz, DMSO-d₆) δ 13.30-12.82 (m, 1H), 8.73 (br s, 1H), 8.64 (s, 2H), 8.00-7.91 (m, 1H), 7.76-7.60 (m, 2H), 7.33-7.22 (m, 1H), 7.12 (br d, J 5.9 Hz, 2H), 6.41 (br s, 1H), 5.42 (br d, J 7.6 Hz, 1H), 4.92-4.82 (m, 1H), 4.77-4.67 (m, 1H), 4.30-4.14 (m, 1H), 4.10-3.97 (m, 1H), 2.49-2.46 (m, 2H), 2.21-1.83 (m, 7H), 1.77 (br dd, J 15.4, 9.0 Hz, 1H), 1.51-1.34 (m, 1H), 0.88 (d, J 6.6 Hz, 6H), 0.57 (s, 9H).

### Example 97: Preparation of Compound 121

### Step 1: 2-Iodo-4-isopropyl-pyrimidine

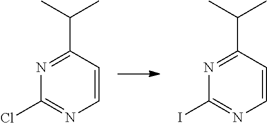

Hydriodic acid (57 wt. % in water) (2 mL of 57% w/v, 8.9124 mmol), precooled to 0° C., was added to solid 2-chloro-4-isopropyl-pyrimidine (500 mg, 3.1926 mmol) in a 10 mL round-bottomed flask. The mixture was kept and vigorously stirred at 0° C. for 2 h. The light brownish green suspension was quickly neutralized at 0° C. with a saturated aqueous solution of potassium carbonate (60 mL), 1N NaOH (8 mL, to reach pH=9) and decolorized with potassium disulfite at 0° C. The aqueous solution was extracted with DCM (5×30 mL), dried over magnesium sulfate, filtered, and evaporated under reduced pressure to give 2-iodo-4-isopropyl-pyrimidine (798 mg, 96%) as a light yellow oil. ¹H NMR (400 MHz, CDCl₃) δ 8.31 (d, J 5.1 Hz, 1H), 7.16 (d, J 5.1 Hz, 1H), 3.05-2.89 (m, 1H), 1.29 (s, 3H), 1.27 (s, 3H). ESI-MS m/z calc. 247.98105, found 249.2 (M+1)⁺; Retention time: 1.71 minutes; LC method X.

### Step 2: 4-Isopropylpyrimidine-2-carbaldehyde

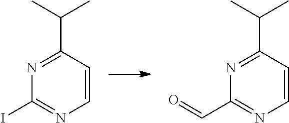

A 10 mL flask equipped with a stirring bar was charged with a solution of 2-iodo-4-isopropyl-pyrimidine (200 mg, 0.8062 mmol) in THE (2.7 mL) under argon atmosphere. isopropylmagnesium chloride lithium chloride complex solution (in THF) (0.7 mL of 1.3 M, 0.9100 mmol) was added dropwise at 0-5° C. The reaction mixture was stirred at 0° C. for 1 h and then Ethyl formate (66.024 mg, 72 μL, 0.8913 mmol) was added. The reaction was allowed to warm to rt and stirred overnight. The mixture was quenched with aqueous saturated NH₄Cl (10 mL) and extracted with DCM (3×20 mL). The combined organic extracts were washed brine (40 mL), dried with sodium sulfate and concentrated under a reduced pressure. The residue was purified by flash chromatography using a 24 g cartridge, eluting with diethyl ether (100%) to give 4-isopropylpyrimidine-2-carbaldehyde (85 mg, 35%) as a colorless oil. ¹H NMR (400 MHz, CDCl₃) δ 10.11 (s, 1H), 8.88-8.84 (m, 1H), 7.38-7.35 (m, 1H), 3.23-3.12 (m, 1H), 1.39-1.37 (m, 3H), 1.37-1.36 (m, 3H). ESI-MS m/z calc. 150.07932, found 151.2 (M+1)⁺; Retention time: 1.15 minutes; LC method X.

### Step 3: 3-[[4-(2,6-Dimethylphenyl)-6-[(2R)-2-[(4-isopropylpyrimidin-2-yl)methylamino]-4,4-dimethyl-pentoxy]pyrimidin-2-yl]sulfamoyl]benzoic acid

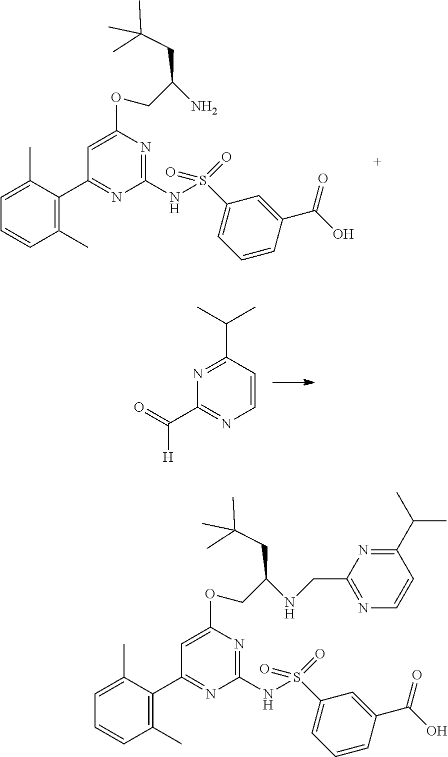 [SMILES: Cc1cccc(C)c1-c1cc(OC[C@H](N)CC(C)(C)C)nc(NS(=O)(=O)c2cccc(C(=O)O)c2)n1 ; CC(C)c1ccnc(C=O)n1 ; Cc1cccc(C)c1-c1cc(OC[C@@H](CC(C)(C)C)NCc2nccc(C(C)C)n2)nc(NS(=O)(=O)c2cccc(C(=O)O)c2)n1]

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (62 mg, 0.1129 mmol) in dichloromethane (1.5 mL) was added a solution of 4-isopropylpyrimidine-2-carbaldehyde (20 mg, 0.1332 mmol) in dichloromethane (1.5 mL), acetic acid (10.560 mg, 10 L, 0.1758 mmol) and the reaction was stirred at room temperature for 45 min. Sodium triacetoxyborohydride (120 mg, 0.5662 mmol) was added and the reaction was stirred at room temperature for 1 h. 1 N aqueous HCl was added (10 mL) and the phases were separated. The aqueous layer was extracted with EtOAc (3×10 mL). The combined organic layers were dried over sodium sulfate, filtered and concentrated under reduced pressure. The residue was co-evaporated with heptanes (2×50 mL). Afforded 3-[[4-(2,6-dimethylphenyl)-6-[(2R)-2-[(4-isopropylpyrimidin-2-yl)methylamino]-4,4-dimethyl-pentoxy]pyrimidin-2-yl]sulfamoyl]benzoic acid (hydrochloride salt) (85 mg, 42%) as a yellow semi-solid. ESI-MS m/z calc. 646.29376, found 647.4 (M+1)⁺; Retention time: 1.49 minutes; LC method X

### Step 4: (11R)-6-(2,6-Dimethylphenyl)-11-(2,2-dimethylpropyl)-12-[(4-isopropylpyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 121)

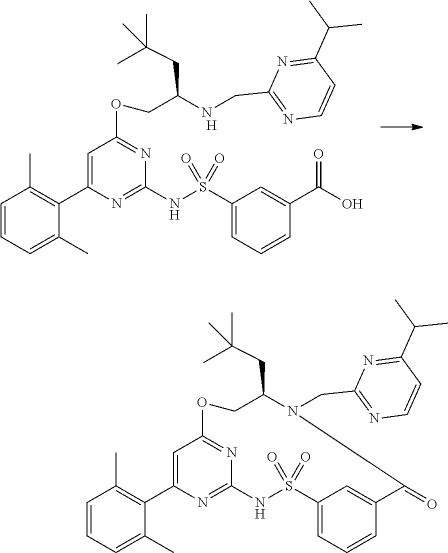

To a 0° C. solution of N-methylmorpholine (92.000 mg, 0.1 mL, 0.9096 mmol) in DMF (12 mL) was added 2-chloro-4,6-dimethoxy-1,3,5-triazine (32 mg, 0.1823 mmol) followed by 3-[[4-(2,6-dimethylphenyl)-6-[(2R)-2-[(4-isopropylpyrimidin-2-yl)methylamino]-4,4-dimethyl-pentoxy]pyrimidin-2-yl]sulfamoyl]benzoic acid (hydrochloride salt) (78 mg, 0.1142 mmol). After 5 min the reaction was stirred at room temperature for 72 h. The reaction mixture was concentrated under reduced pressure at 50° C. The remaining crude was diluted with DCM (50 mL) and the solution was washed with a 1:1 v/v mix of water and brine (4×15 mL), water (15 mL) and brine (40 mL). The resulting organic layer was dried over sodium sulfate, filtered and concentrated under reduced pressure. The crude was purified by flash chromatography using a 12 g cartridge, eluting with a gradient of EtOAc in DCM (5 to 50% in 25 CV). Removal of the volatiles under reduced pressure afforded a white solid which was purified by reverse phase chromatography on a 15.5 g C₁₈ cartridge using a gradient of MeCN in acidic water (0.1% v/v of formic acid in water) of 40 to 100% for 15 CV then 100% for 5 CV. The fractions containing the product were evaporated and then lyophilized. Afforded (11R)-6-(2,6-dimethylphenyl)-11-(2,2-dimethylpropyl)-12-[(4-isopropylpyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (8.6 mg, 12%) as a white solid. ESI-MS m/z calc. 628.2832, found 629.3 (M+1)⁺; Retention time: 4.47 minutes; LC method Y. ¹H NMR (400 MHz, DMSO-d₆) δ 13.28-12.78 (m, 1H), 8.90-8.78 (m, 1H), 8.71-8.63 (m, 1H), 8.02-7.89 (m, 1H), 7.77-7.59 (m, 2H), 7.37-7.22 (m, 2H), 7.19-7.04 (m, 2H), 6.48-6.34 (m, 1H), 5.61-5.42 (m, 1H), 4.92-4.81 (m, 1H), 4.77-4.65 (m, 1H), 4.31-4.13 (m, 1H), 4.09-3.94 (m, 1H), 3.08-2.94 (m, 1H), 2.21-1.87 (m, 6H), 1.84-1.69 (m, 1H), 1.47-1.37 (m, 1H), 1.28 (d, J 7.1 Hz, 3H), 1.24 (d, J=6.8 Hz, 3H), 0.58 (s, 9H).

### Example 98: Preparation of Compound 122

### Step 1: 3-[[4-(2,6-dimethylphenyl)-6-[(2R)-4,4-dimethyl-2-[[4-(trifluoromethyl)pyrimidin-2-yl]methylamino]pentoxy]pyrimidin-2-yl]sulfamoyl]benzoic acid

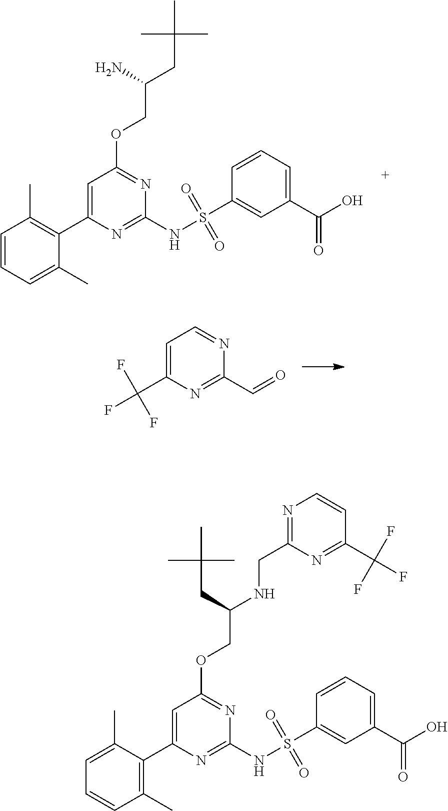 [SMILES: Cc1cccc(C)c1-c1cc(OC[C@H](N)CC(C)(C)C)nc(NS(=O)(=O)c2cccc(C(=O)O)c2)n1 ; O=Cc1nccc(C(F)(F)F)n1 ; Cc1cccc(C)c1-c1cc(OC[C@@H](CC(C)(C)C)NCc2nccc(C(F)(F)F)n2)nc(NS(=O)(=O)c2cccc(C(=O)O)c2)n1]

In a 4 mL vial, to a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (60 mg, 0.1093 mmol) and 4-(trifluoromethyl)pyrimidine-2-carbaldehyde (20 mg, 0.1136 mmol) in anhydrous dichloromethane (0.6 mL) were added glacial acetic acid (10 μL, 0.1758 mmol), DIEA (100 μL, 0.5741 mmol) and sodium triacetoxyborohydride (140 mg, 0.6606 mmol), in that order. The vial was purged with nitrogen briefly and capped and allowed to stir at ambient temperature for 40 min. Then methanol (0.3 mL) and water (0.2 mL) were added to the reaction and the volatiles were removed under reduced pressure and the residue was taken up in DMSO (1.5 mL), micro-filtered, and purified by reverse-phase HPLC (1-99% acetonitrile in water over 15 min, HCl as modifier) to furnish 3-[[4-(2,6-dimethylphenyl)-6-[(2R)-4,4-dimethyl-2-[[4-(trifluoromethyl)pyrimidin-2-yl]methylamino]pentoxy]pyrimidin-2-yl]sulfamoyl]benzoic acid (hydrochloride salt) (28 mg, 36%) as white solid. ESI-MS m/z calc. 672.2342, found 673.2 (M+1)⁺; Retention time: 1.28 minutes; LC method A. ¹H NMR (400 MHz, DMSO-d₆) δ 13.5 (s, 1H), 9.52 (s, 2H), 9.29 (d, J 5.1 Hz, 1H), 8.45 (t, J 1.8 Hz, 1H), 8.22-8.05 (m, 3H), 7.69 (t, J=7.8 Hz, 1H), 7.25 (d, J 8.3 Hz, 1H), 7.13 (d, J 7.6 Hz, 2H), 6.31 (s, 1H), 4.71 (s, 2H), 4.49-4.38 (m, 1H), 4.31 (s, 1H), 3.73 (s, 1H), 2.01 (s, 6H), 1.79-1.61 (m, 2H), 0.93 (s, 9H).

### Step 2: (11R)-6-(2,6-Dimethylphenyl)-11-(2,2-dimethylpropyl)-12-{[4-(trifluoromethyl)pyrimidin-2-yl]methyl}-9-oxa-2λ⁶-thia-3,5,12,19-tetraazatricyclo[12.3.1.14,8]nonadeca-1(17),4,6,8(19),14(18),15-hexaene-2,2,13-trione (Compound 122)

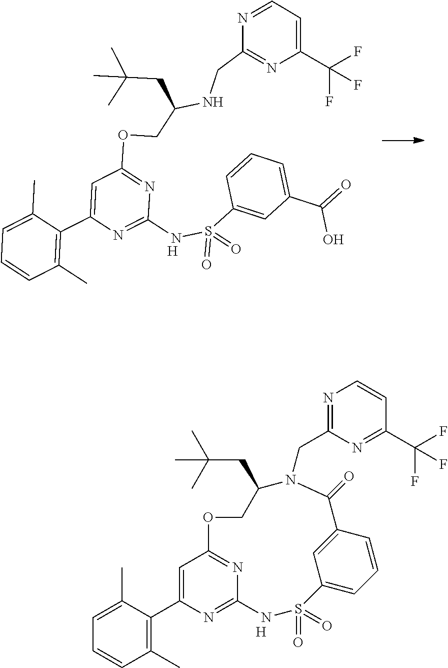

To a stirred solution of 3-[[4-(2,6-dimethylphenyl)-6-[(2R)-4,4-dimethyl-2-[[4-(trifluoromethyl)pyrimidin-2-yl]methylamino]pentoxy]pyrimidin-2-yl]sulfamoyl]benzoic acid (hydrochloride salt) (29 mg, 0.04089 mmol) in anhydrous DMF (1.5 mL) was added 2-chloro-4,6-dimethoxy-1,3,5-triazine (12 mg, 0.06835 mmol) (CDMT), followed by addition of 4-methylmorpholine (30 μL, 0.2729 mmol) at 0-4° C. (ice-water bath) under nitrogen. The clear reaction was allowed to stir at that temperature for 15 min, then allowed to stir at room temperature for h. The product was purified by preparative reverse-phase HPLC (1-99% acetonitrile in water over 15 min, HCl as modifier) to furnish (11R)-6-(2,6-dimethylphenyl)-11-(2,2-dimethylpropyl)-12-{[4-(trifluoromethyl)pyrimidin-2-yl]methyl}-9-oxa-2λ⁶-thia-3,5,12,19-tetraazatricyclo[12.3.1.14,8]nonadeca-1(17),4,6,8(19),14(18),15-hexaene-2,2,13-trione (13 mg, 48%) as a white solid. ESI-MS m/z calc. 654.22363, found 655.4 (M+1)⁺; Retention time: 1.79 minutes; LC method A. ¹H NMR (400 MHz, DMSO-d₆) δ 13.01 (s, 1H), 9.21 (d, J 5.0 Hz, 1H), 8.73 (s, 1H), 7.96 (d, J 7.2 Hz, 1H), 7.93 (d, J 5.1 Hz, 1H), 7.79-7.66 (m, 1H), 7.64 (s, 1H), 7.26 (t, J 7.6 Hz, 1H), 7.12 (d, J 7.7 Hz, 2H), 6.41 (s, 1H), 5.43 (dd, J 10.8, 4.4 Hz, 1H), 5.04-4.84 (m, 2H), 4.27 (t, J 11.2 Hz, 1H), 4.08-3.97 (m, 1H), 1.96 (s, 6H), 1.79 (dd, J 15.2, 8.8 Hz, 1H), 1.44 (d, J 14.9 Hz, 1H), 0.58 (s, 9H).

### Example 99: Preparation of Compound 123

### Step 1: 3-[[4-[(2R)-2-[(4-Chloropyrimidin-2-yl)methylamino]-4,4-dimethyl-pentoxy]-6-(2,6-dimethylphenyl)pyrimidin-2-yl]sulfamoyl]benzoic acid

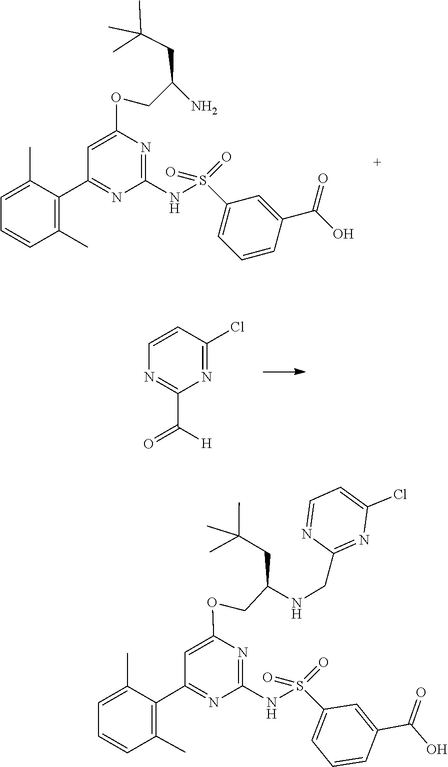 [SMILES: Cc1cccc(C)c1-c1cc(OC[C@H](N)CC(C)(C)C)nc(NS(=O)(=O)c2cccc(C(=O)O)c2)n1 ; O=Cc1nccc(Cl)n1 ; Cc1cccc(C)c1-c1cc(OC[C@@H](CC(C)(C)C)NCc2nccc(Cl)n2)nc(NS(=O)(=O)c2cccc(C(=O)O)c2)n1]

To a solution of 4-chloropyrimidine-2-carbaldehyde (50 mg, 0.3087 mmol) in dichloromethane (2 mL) and Acetic acid (0.2 mL) at 0° C. was added 3-[[4-[(2R)-2-amino-4,4-dimethyl-pentoxy]-6-(2,6-dimethylphenyl)pyrimidin-2-yl]sulfamoyl]benzoic acid (hydrochloride salt) (170 mg, 0.2941 mmol) in solid form and by small portion. The reaction was stirred at 0° C. for 15 min and at room temperature for 1 h. Sodium triacetoxyborohydride (315 mg, 1.4863 mmol) was added and the reaction was stirred at room temperature for 1.5 h. Then, 1 N aqueous HCl was added (10 mL) and the phases were separated. The aqueous layer was extracted with EtOAc (3×10 mL). The combined organic layers were dried over sodium sulfate, filtered and concentrated under reduced pressure. The residue was co-evaporated with heptanes (2×25 mL) to remove residual acetic acid. Afforded 3-[[4-[(2R)-2-[(4-chloropyrimidin-2-yl)methylamino]-4,4-dimethyl-pentoxy]-6-(2,6-dimethylphenyl)pyrimidin-2-yl]sulfamoyl]benzoic acid (hydrochloride salt) (202 mg, 82%) as an off-white solid. ESI-MS m z calc. 638.2078, found 639.2 (M+1)⁺; Retention time: 2.62 minutes; LC method Y.

### Step 2: (11R)-12-[(4-Chloropyrimidin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

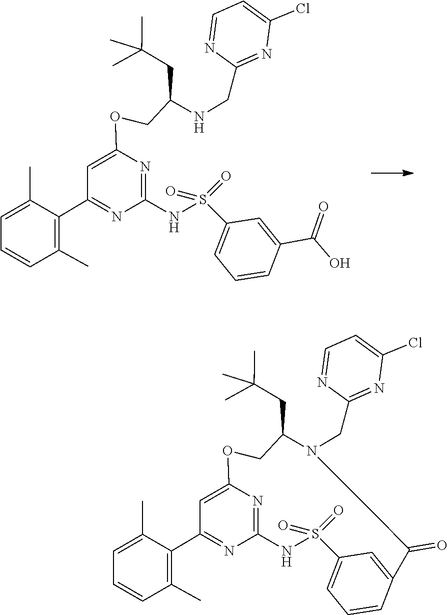 [SMILES: Cc1cccc(C)c1-c1cc(OC[C@@H](CC(C)(C)C)NCc2nccc(Cl)n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1nccc(Cl)n1)[C@H](CC(C)(C)C)CO2]

To a 0° C. solution of N-methylmorpholine (791.20 mg, 0.86 mL, 7.8223 mmol) in DMF (70 mL) was added 2-chloro-4,6-dimethoxy-1,3,5-triazine (162 mg, 0.9227 mmol) followed by 3-[[4-[(2R)-2-[(4-chloropyrimidin-2-yl)methylamino]-4,4-dimethyl-pentoxy]-6-(2,6-dimethylphenyl)pyrimidin-2-yl]sulfamoyl]benzoic acid (hydrochloride salt) (685 mg, 0.6002 mmol). After 5 min the reaction was stirred at room temperature for 16 h. The reaction mixture was concentrated under reduced pressure at 50° C. The remaining crude was diluted with DCM (50 mL) and the solution was washed with a 1:1 v/v mix of water and brine (4×20 mL), water (25 mL) and brine (50 mL). The resulting organic layer was dried over sodium sulfate, filtered and concentrated under reduced pressure. The crude was purified by reverse phase chromatography on a 50 g C₁₈ cartridge using a gradient of MeCN in acidic water (0.1% v/v of formic acid in water) of 5 to 100% for 20 CV. The fractions containing the product were evaporated. Afforded (11R)-12-[(4-chloropyrimidin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (250 mg, 67%) as a white solid. ESI-MS m/z calc. 620.19727, found 621.3 (M+1)⁺; Retention time: 4.24 minutes; LC method Y.

### Step 3: (11R)-6-(2,6-Dimethylphenyl)-11-(2,2-dimethylpropyl)-12-[[4-[isopropyl(methyl)amino]pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 123)

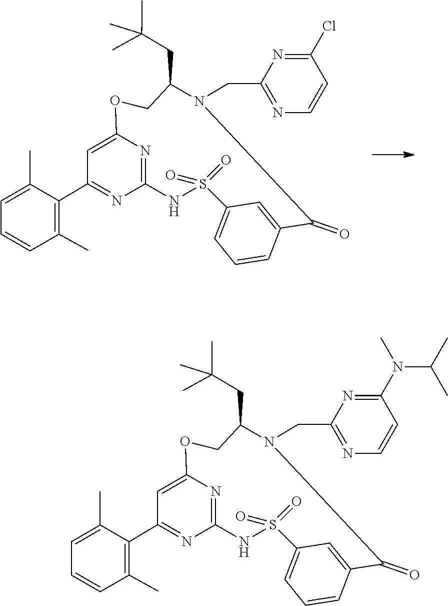 [SMILES: Cc1cccc(C)c1-c1cc2nc(n1)NS(=O)(=O)c1cccc(c1)C(=O)N(Cc1nccc(Cl)n1)[C@H](CC(C)(C)C)CO2 ; Cc1cccc(C)c1-c1cc2nc(n1)NS(=O)(=O)c1cccc(c1)C(=O)N(Cc1nccc(N(C)C(C)C)n1)[C@H](CC(C)(C)C)CO2]

To a stirred solution of (11R)-12-[(4-chloropyrimidin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (10 mg, 0.0146 mmol) in tetrahydrofuran (0.1 mL) was added N-methylpropan-2-amine (7.0200 mg, 10 μL, 0.0960 mmol) and triethylamine (7.2600 mg, 10 μL, 0.0717 mmol). The mixture was stirred at room temperature for 24 hours then tetrahydrofuran (0.1 mL) was added. The mixture was stirred 24 hours at room temperature. The volatiles were removed in vacuo and the crude material was purified by reverse phase chromatography on a 15.5 g C₁₈ cartridge using a gradient of MeOH in acidic water (0.1% v/v of formic acid in water) (5% for 5 CV then 5 to 100% for 20 CV). Afforded after evaporation and lyophilization (11R)-6-(2,6-dimethylphenyl)-11-(2,2-dimethylpropyl)-12-[[4-[isopropyl(methyl)amino]pyrimidin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (6.6 mg, 68%) as a white solid. ESI-MS m/z calc. 657.30975, found 658.4 (M+1)⁺; Retention time: 2.74 minutes; LC method Y. ¹H NMR (400 MHz, DMSO-d₆) δ 13.04-12.53 (m, 1H), 8.78 (s, 1H), 8.13 (d, J 6.1 Hz, 1H), 7.95 (br d, J 6.6 Hz, 1H), 7.72-7.63 (m, 2H), 7.28-7.23 (m, 1H), 7.12 (br d, J=7.6 Hz, 2H), 6.54-6.46 (m, 1H), 6.42 (br s, 1H), 5.46-5.39 (m, 1H), 4.66 (d, J 16.9 Hz, 1H), 4.45 (d, J 16.9 Hz, 1H), 4.25-4.15 (m, 1H), 4.12-4.04 (m, 1H), 2.86 (br s, 3H), 2.19-1.84 (m, 6H), 1.77-1.70 (m, 1H), 1.38 (d, J 14.7 Hz, 1H), 1.19 (d, J 6.6 Hz, 3H), 1.08 (br d, J 6.4 Hz, 3H), 0.55 (s, 9H).

### Example 100: Preparation of Compound 124

### Step 1: 3-[(4-{[(2R)-4,4-Dimethyl-2-{[(pyridin-2-yl)methyl]amino}pentyl]oxy}-6-(2,6-dimethylphenyl)pyrimidin-2-yl)sulfamoyl]benzoic acid

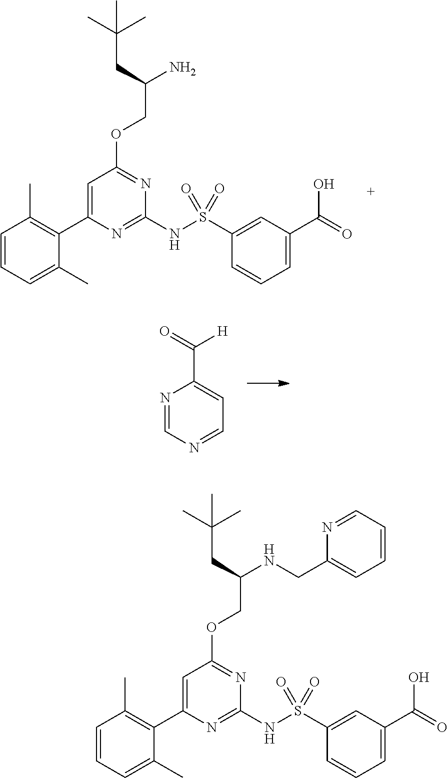 [SMILES: Cc1cccc(C)c1-c1cc(OC[C@H](N)CC(C)(C)C)nc(NS(=O)(=O)c2cccc(C(=O)O)c2)n1 ; O=Cc1ccncn1 ; Cc1cccc(C)c1-c1cc(OC[C@@H](CC(C)(C)C)NCc2ccccn2)nc(NS(=O)(=O)c2cccc(C(=O)O)c2)n1]

3-[[4-[(2R)-2-Amino-4,4-dimethyl-pentoxy]-6-(2,6-dimethylphenyl)pyrimidin-2-yl]sulfamoyl]benzoic acid (hydrochloride salt) (50 mg, 0.09106 mmol), pyridine-2-carbaldehyde (13 μL, 0.1367 mmol), and sodium triacetoxyborohydride (approximately 77.19 mg, 0.3642 mmol) were combined in dichloromethane and stirred at room temperature for 2 h. The reaction mixture was partitioned between ethyl acetate and a 1M HCl solution. The organics were separated, washed with brine, dried over sodium sulfate and evaporated. The crude material was purified by reverse phase HPLC utilizing a gradient of 1-99% acetonitrile in 5 mM aqueous HCl to yield 3-[(4-{[(2R)-4,4-dimethyl-2-{[(pyridin-2-yl)methyl]amino}pentyl]oxy}-6-(2,6-dimethylphenyl)pyrimidin-2-yl)sulfamoyl]benzoic acid (hydrochloride salt) (12.2 mg, 22%). ESI-MS m/z calc. 603.2515, found 604.5 (M+1)⁺; Retention time: 0.52 minutes; LC method D.

### Step 2: (11R)-6-(2,6-Dimethylphenyl)-11-(2,2-dimethylpropyl)-12-[(pyridin-2-yl)methyl]-9-oxa-2λ⁶-thia-3,5,12,19-tetraazatricyclo[12.3.1.14,8]nonadeca-1(17),4,6,8(19),14(18),15-hexaene-2,2,13-trione (Compound 124)

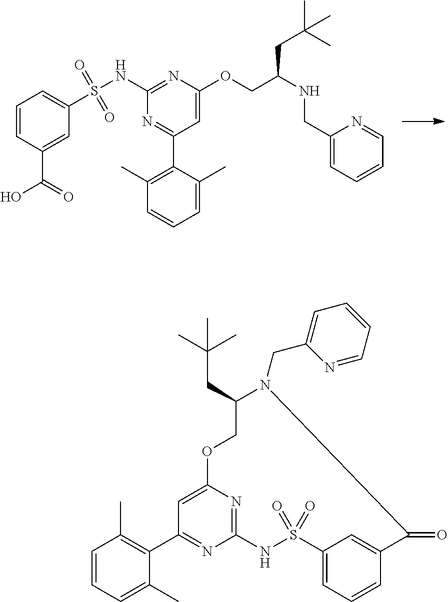 [SMILES: Cc1cccc(C)c1-c1cc(OC[C@@H](CC(C)(C)C)NCc2ccccn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccccn1)[C@H](CC(C)(C)C)CO2]

3-[(4-{[(2R)-4,4-Dimethyl-2-{[(pyridin-2-yl)methyl]amino}pentyl]oxy}-6-(2,6-dimethylphenyl)pyrimidin-2-yl)sulfamoyl]benzoic acid (hydrochloride salt) (12.2 mg, 0.02021 mmol), HATU (10.6 mg, 0.02788 mmol), and triethylamine (20 μL, 0.1435 mmol) were combined in DMF (1 mL) and stirred at room temperature for 16 h. The reaction mixture was filtered and purified by reverse phase HPLC utilizing a gradient of 1-99% acetonitrile in 5 mM aqueous HCl to yield (11R)-6-(2,6-dimethylphenyl)-11-(2,2-dimethylpropyl)-12-[(pyridin-2-yl)methyl]-9-oxa-2λ⁶-thia-3,5,12,19-tetraazatricyclo[12.3.1.14,8]nonadeca-1(17),4,6,8(19),14(18),15-hexaene-2,2,13-trione (hydrochloride salt) (4.8 mg, 41%). ESI-MS m/z calc. 585.24097, found 586.2 (M+1)⁺; Retention time: 1.44 minutes; LC method A.

### Example 101: Preparation of Compound 125

### Step 1: tert-Butyl N-[6-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]-2-pyridyl]carbamate

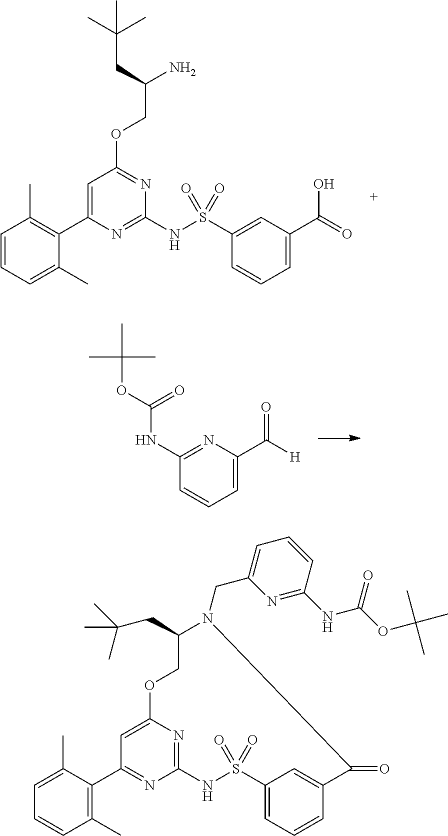 [SMILES: Cc1cccc(C)c1-c1cc(OC[C@H](N)CC(C)(C)C)nc(NS(=O)(=O)c2cccc(C(=O)O)c2)n1 ; CC(C)(C)OC(=O)Nc1cccc(C=O)n1 ; Cc1cccc(C)c1-c1cc2nc(n1)NS(=O)(=O)c1cccc(c1)C(=O)N(Cc1cccc(NC(=O)OC(C)(C)C)n1)[C@H](CC(C)(C)C)CO2]

3-[[4-[(2R)-2-Amino-4,4-dimethyl-pentoxy]-6-(2,6-dimethylphenyl)pyrimidin-2-yl]sulfamoyl]benzoic acid (hydrochloride salt) (100 mg, 0.1821 mmol) was combined with the tert-butyl N-(6-formyl-2-pyridyl)carbamate (61 mg, 0.2745 mmol) in DCM (0.5 mL) and sodium triacetoxyborohydride (116 mg, 0.5473 mmol) was added in two portions over 15 minutes. The reaction was then stirred at room temperature for one hour. Upon completion, the reaction mixture was partitioned between 1M HCl and ethyl acetate. The layers were separated and the aqueous was extracted an additional 2× with ethyl acetate. The combined organics were washed with brine, dried over sodium sulfate, and concentrated. The product in DMF (4 mL) was added at a rapid dropwise to a stirring solution of HATU (140 mg, 0.3682 mmol) and DIPEA (approximately 141.3 mg, 190.4 μL, 1.093 mmol) in DMF (2 mL). The reaction mixture was stirred at room temperature for 16 hours, then was partially concentrated, and partitioned between aqueous 0.5M HCl and ethyl acetate. The layers were separated and the aqueous was extracted an additional 2× with ethyl acetate. The combined organics were washed with brine, dried over sodium sulfate and concentrated. The resulting crude material purified by chromatography on silica gel, eluting with a gradient of 0-100% ethyl acetate in hexanes. Fractions containing product were then concentrated to give the tert-butyl N-[6-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]-2-pyridyl]carbamate (40 mg, 31%) products. ESI-MS m/z calc. 700.3043, found 701.6 (M+1)⁺; Retention time: 0.76 minutes; LC method D.

### Step 2: (11R)-12-[(6-Amino-2-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 125)

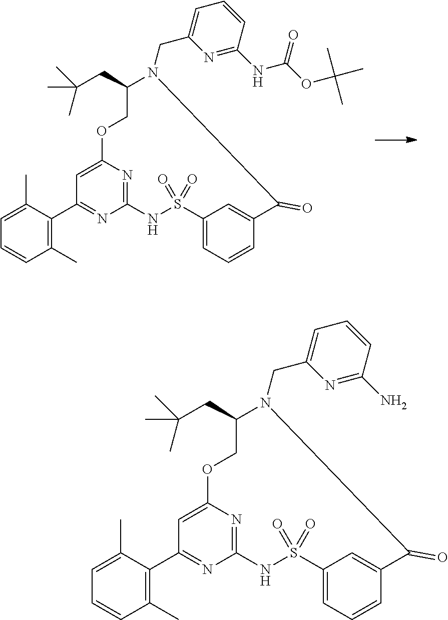 [SMILES: Cc1cccc(C)c1-c1cc2nc(n1)NS(=O)(=O)c1cccc(c1)C(=O)N(Cc1cccc(NC(=O)OC(C)(C)C)n1)[C@H](CC(C)(C)C)CO2 ; Cc1cccc(C)c1-c1cc2nc(n1)NS(=O)(=O)c1cccc(c1)C(=O)N(Cc1cccc(N)n1)[C@H](CC(C)(C)C)CO2]

tert-butyl N-[6-[[(11R)-6-(2,6-Dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]-2-pyridyl]carbamate (18 mg, 0.02568 mmol) was combined with HCl (300 μL of 4 M, 1.200 mmol) in dioxane in a screw cap vial and heated to 50° C. for two hours. The compound was then cooled to room temperature, concentrated, then dissolved in 1:1 DMSO/methanol, filtered, and purified by reverse phase HPLC (1-60ACN in water, HCl modifier, 15 min run) to give (11R)-12-[(6-amino-2-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (7.1 mg, 43%). ESI-MS m/z calc. 600.2519, found 601.6 (M+1)⁺; Retention time: 1.22 minutes; LC method A.

### Example 102: Preparation of Compound 126

### Step 1: 3-[[4-(2,6-Dimethylphenyl)-6-[(2R)-2-[(6-fluoro-2-pyridyl)methylamino]-4,4-dimethyl-pentoxy]pyrimidin-2-yl]sulfamoyl]benzoic acid

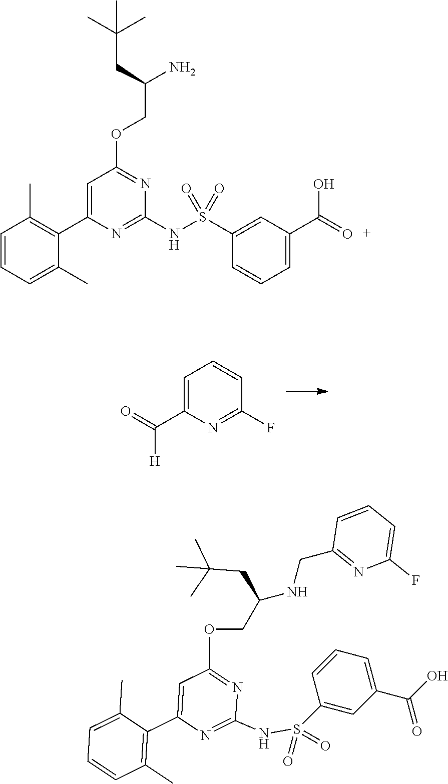

In a 20 mL vial, to a stirred heterogeneous mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (500 mg, 0.9106 mmol) in anhydrous dichloromethane (4 mL) were added 6-fluoropyridine-2-carbaldehyde (115 mg, 0.9193 mmol), and glacial acetic acid (60 μL, 1.055 mmol), in that order. The vial was purged with nitrogen briefly and capped and stirred for 5 min, then sodium triacetoxyborohydride (970 mg, 4.577 mmol) was added, followed by addition of DIEA (500 μL, 2.871 mmol), and the capped vial was allowed to stir at ambient temperature for 25 min. Then the mixture was poured over ice-cold water containing hydrochloric acid (10 mL of 1.0 M, 10.00 mmol) (to pH about 2) and extracted with ethyl acetate (3×30 mL). The combined organics were washed successively with water (20 mL), brine (20 mL), dried over sodium sulfate, filtered and concentrated under reduced pressure to furnish crude 3-[[4-(2,6-dimethylphenyl)-6-[(2R)-2-[(6-fluoro-2-pyridyl)methylamino]-4,4-dimethyl-pentoxy]pyrimidin-2-yl]sulfamoyl]benzoic acid (Dihydrochloride salt) (607 mg, 96%) as an off-white solid. The crude was used in the subsequent cyclization step. Following the above protocol, using same quantities another lot of crude was obtained. The crude was purified by preparative reverse-phase HPLC (1-99% acetonitrile in water over 15 min, 5 mM HCl as modifier) to furnish 3-[[4-(2,6-dimethylphenyl)-6-[(2R)-2-[(6-fluoro-2-pyridyl)methylamino]-4,4-dimethyl-pentoxy]pyrimidin-2-yl]sulfamoyl]benzoic acid (Dihydrochloride salt) (387 mg, 61%) a white solid. ESI-MS m/z calc. 621.2421, found 622.4 (M+1)⁺; Retention time: 1.29 minutes; LC method A. ESI-MS m/z calc. 621.2421, found 622.4 (M+1)⁺; Retention time: 0.59 minutes; LC method D. ¹H NMR (400 MHz, DMSO-d₆) δ 13.3 (s, 1H), 9.47 (s, 1H), 9.41 (s, 1H), 8.44 (d, J 1.9 Hz, 1H), 8.23-8.03 (m, 3H), 7.69 (t, J 7.8 Hz, 1H), 7.55 (dd, J 7.4, 2.3 Hz, 1H), 7.31-7.20 (m, 2H), 7.19-7.09 (m, 2H), 6.34 (s, 1H), 4.49-4.34 (m, 3H), 4.29 (dd, J 12.6, 6.0 Hz, 1H), 3.59 (s, 1H), 2.02 (s, 6H), 1.78-1.59 (m, 2H), 0.92 (s, 9H).

### Step 2: (11R)-6-(2,6-Dimethylphenyl)-11-(2,2-dimethylpropyl)-12-[(6-fluoro-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

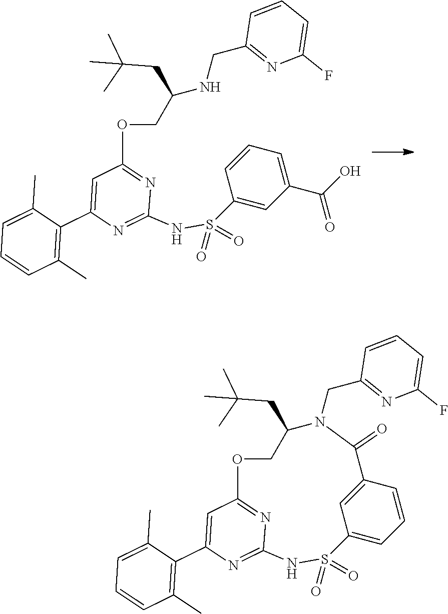 [SMILES: Cc1cccc(C)c1-c1cc(OC[C@@H](CC(C)(C)C)NCc2cccc(F)n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cccc(F)n1)[C@H](CC(C)(C)C)CO2]

To a stirred solution of 3-[[4-(2,6-dimethylphenyl)-6-[(2R)-2-[(6-fluoro-2-pyridyl)methylamino]-4,4-dimethyl-pentoxy]pyrimidin-2-yl]sulfamoyl]benzoic acid (Dihydrochloride salt) (700 mg, 1.008 mmol) in anhydrous DMF (40 mL) was added 2-chloro-4,6-dimethoxy-1,3,5-triazine (215 mg, 1.225 mmol) (CDMT), followed by addition of 4-methylmorpholine (600 μL, 5.457 mmol) at 0-5° C. (ice-water bath under nitrogen. After stirring for 30 min, the reaction was allowed to warm to ambient temperature and stirring continued overnight (total 14 h). The volatiles were removed under reduced pressure and the residue was purified by preparative reverse-phase HPLC (1-99% acetonitrile in water over 15 min, 5 mM HCl as modifier) to give (11R)-6-(2,6-dimethylphenyl)-11-(2,2-dimethylpropyl)-12-[(6-fluoro-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (403 mg, 66%) as a white solid. It was used in the subsequent reactions. ESI-MS m/z calc. 603.23157, found 604.3 (M+1)⁺; Retention time: 1.79 minutes; LC method A. ¹H NMR (400 MHz, DMSO-d₆) δ 13.07 (s, 1H), 8.65 (s, 1H), 8.08-7.90 (m, 2H), 7.68 (s, 2H), 7.41 (dd, J 7.5, 2.4 Hz, 1H), 7.29-7.22 (m, 1H), 7.19-7.10 (m, 2H), 7.06 (dd, J 8.1, 2.6 Hz, 1H), 6.43 (s, 1H), 5.35 (dd, J 11.0, 4.3 Hz, 1H), 4.80 (d, J 16.1 Hz, 1H), 4.59 (d, J 16.1 Hz, 1H), 4.26 (t, J 11.2 Hz, 1H), 4.11-3.91 (m, 1H), 2.25-1.84 (m, 6H), 1.78 (dd, J 15.2, 8.8 Hz, 1H), 1.41 (d, J 15.1 Hz, 1H), 0.55 (s, 9H).

### Step 3: (11R)-6-(2,6-Dimethylphenyl)-11-(2,2-dimethylpropyl)-2,2-dioxo-12-[(6-phenoxy-2-pyridyl)methyl]-9-oxa-2λ⁶-thia-3,5,12,19-tetrazatricyclo[12.3.1.14,8]nonadeca-1(18),4(19),5,7,14,16-hexaen-13-one (Compound 126)

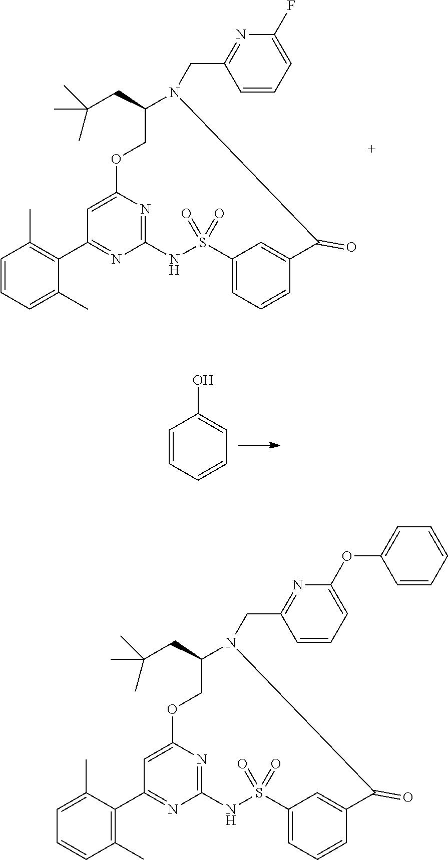 [SMILES: Cc1cccc(C)c1-c1cc2nc(n1)NS(=O)(=O)c1cccc(c1)C(=O)N(Cc1cccc(F)n1)[C@H](CC(C)(C)C)CO2 ; "+" ; Oc1ccccc1 ; Cc1cccc(C)c1-c1cc2nc(n1)NS(=O)(=O)c1cccc(c1)C(=O)N(Cc1cccc(Oc3ccccc3)n1)[C@H](CC(C)(C)C)CO2]

(11R)-6-(2,6-Dimethylphenyl)-11-(2,2-dimethylpropyl)-12-[(6-fluoro-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (15 mg, 0.02485 mmol) was combined with phenol (approximately 7.016 mg, 6.619 μL, 0.07455 mmol) and potassium carbonate (approximately 20.61 mg, 0.1491 mmol) in DMF (0.25 mL) in a screwcap vial, and heated to 120° C. After 5 hours DMSO (0.25 mL) and additional portions of phenol (approximately 7.016 mg, 6.619 μL, 0.07455 mmol) and potassium carbonate (approximately 20.61 mg, 0.1491 mmol) were added, and the reaction was stirred for an additional 14 hours. The reaction mixture was then cooled to room temperature, filtered, and purified by reverse phase HPLC (1-70% ACN in water, HCl modifier, 15 min run) to give upon drying, (11R)-6-(2,6-dimethylphenyl)-11-(2,2-dimethylpropyl)-2,2-dioxo-12-[(6-phenoxy-2-pyridyl)methyl]-9-oxa-2λ⁶-thia-3,5,12,19-tetrazatricyclo[12.3.1.14,8]nonadeca-1(18),4(19),5,7,14,16-hexaen-13-one (10.9 mg, 64%) ESI-MS m/z calc. 677.2672, found 678.3 (M+1)⁺; Retention time: 2.1 minutes; LC method A.

### Example 103: Preparation of Compound 127

### Step 1: (11R)-6-(2,6-Dimethylphenyl)-11-(2,2-dimethylpropyl)-12-[[6-[3-methoxypropyl(methyl)amino]-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 127)

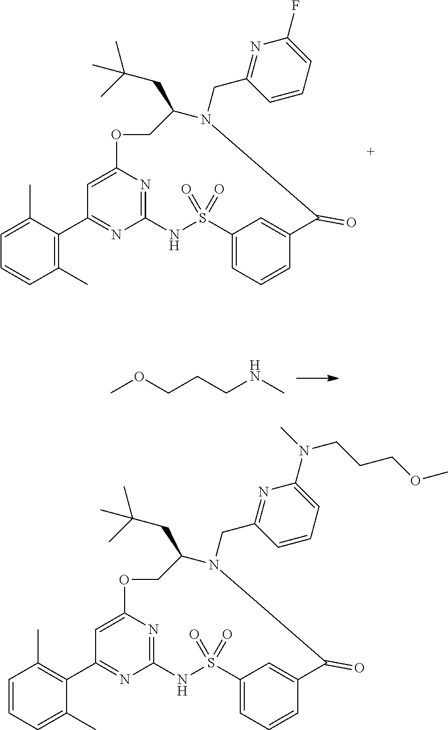

(11R)-6-(2,6-Dimethylphenyl)-11-(2,2-dimethylpropyl)-12-[(6-fluoro-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (15 mg, 0.02485 mmol) was combined with 3-methoxy-N-methyl-propan-1-amine (approximately 12.81 mg, 0.1242 mmol) and potassium carbonate (approximately 17.18 mg, 0.1243 mmol) in DMSO (0.2 mL) and heated to 120° C. in a screwcap vial. After the indicated time the reaction was cooled to room temperature, diluted with methanol, filtered, and purified by reverse phase HPLC (1-99 ACN in water, HCl modifier, 15 min run) to give the indicated (11R)-6-(2,6-dimethylphenyl)-11-(2,2-dimethylpropyl)-12-[[6-[3-methoxypropyl(methyl)amino]-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (8.1 mg, 47%). ESI-MS m/z calc. 686.325, found 687.5 (M+1)⁺; Retention time: 1.49 minutes; LC method A.

### Example 104: Preparation of Compound 128

### Step 1: (11R)-12-[[6-[Bis(2-methoxyethyl)amino]-2-pyridyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 128)

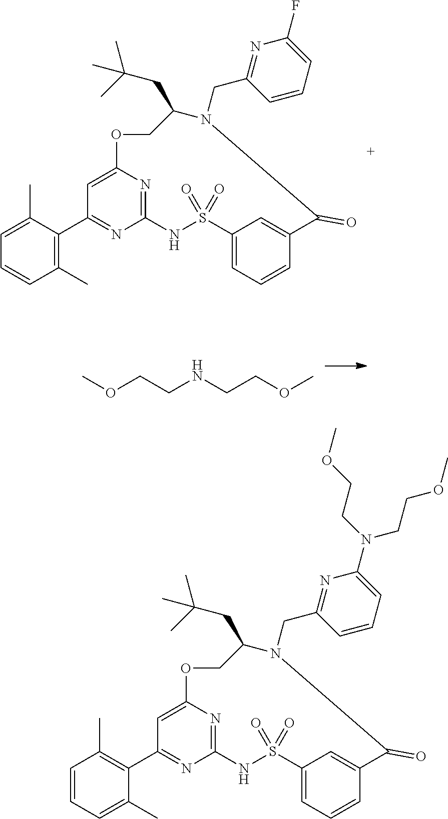

(11R)-6-(2,6-Dimethylphenyl)-11-(2,2-dimethylpropyl)-12-[(6-fluoro-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (15 mg, 0.02485 mmol) was combined with 2-methoxy-N-(2-methoxyethyl)ethanamine (approximately 16.54 mg, 0.1242 mmol) and potassium carbonate (approximately 17.18 mg, 0.1243 mmol) in DMSO (0.2 mL) and heated to 120° C. in a screwcap vial. After the indicated time the reaction was cooled to room temperature, diluted with methanol, filtered, and purified by reverse phase HPLC (1-99 ACN in water, HCl modifier, 15 min run) to give the indicated (11R)-12-[[6-[bis(2-methoxyethyl)amino]-2-pyridyl]methyl]-6-(2,6-dimethylphenyl)-11-(2,2-dimethylpropyl)-2,2-dioxo-9-oxa-2λ⁶-thia-3, 5,12,19-tetrazatricyclo[12.3.1.14,8]nonadeca-1(18),4(19),5,7,14,16-hexaen-13-one (3.4 mg, 19%). ESI-MS m/z calc. 716.33563, found 717.5 (M+1)⁺; Retention time: 1.56 minutes; LC method A.

### Example 105: Preparation of Compound 129

### Step 1: (11R)-6-(2,6-Dimethylphenyl)-11-(2,2-dimethylpropyl)-12-[[6-[2-(methoxymethyl)pyrrolidin-1-yl]-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 129)

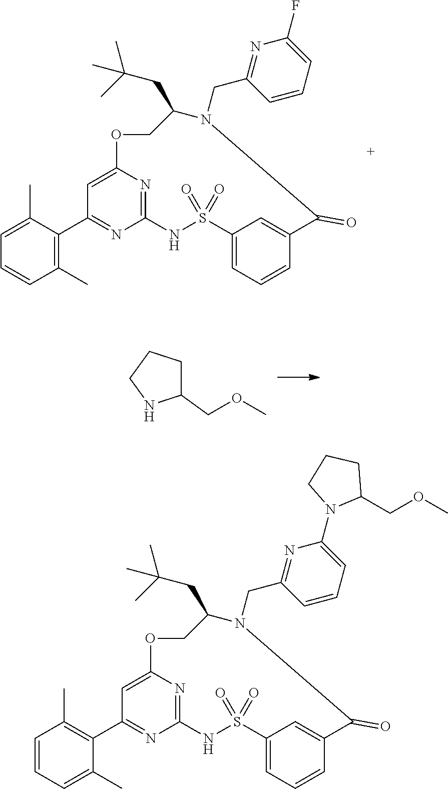

(11R)-6-(2,6-Dimethylphenyl)-11-(2,2-dimethylpropyl)-12-[(6-fluoro-2-pyridyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (15 mg, 0.02485 mmol) was combined with 2-(methoxymethyl)pyrrolidine (approximately 14.30 mg, 0.1242 mmol) and potassium carbonate (approximately 17.17 mg, 0.1242 mmol) in DMSO (0.2 mL) and heated to 120° C. in a screwcap vial. After the indicated time the reaction was cooled to room temperature, diluted with methanol, filtered, and purified by reverse phase HPLC (1-99 ACN in water, HCl modifier, 15 min run) to give the indicated (11R)-6-(2,6-dimethylphenyl)-11-(2,2-dimethylpropyl)-12-[[6-[2-(methoxymethyl)pyrrolidin-1-yl]-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (4.8 mg, 27%). ESI-MS m/z calc. 698.325, found 699.5 (M+1)⁺; Retention time: 1.53 minutes; LC method A.

### Example 106: Preparation of Compound 130

### Step 1: (11R)-6-(2,6-Dimethylphenyl)-11-(2,2-dimethylpropyl)-12-[[6-(methoxymethyl)-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 130)

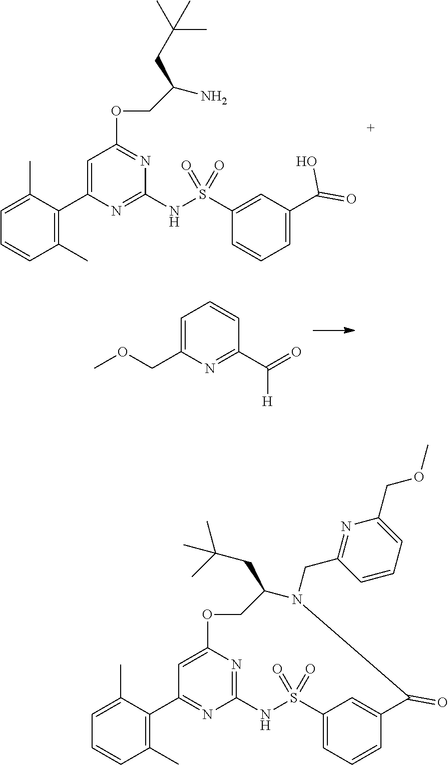 [SMILES: Cc1cccc(C)c1-c1cc(OC[C@H](N)CC(C)(C)C)nc(NS(=O)(=O)c2cccc(C(=O)O)c2)n1 ; "+" ; COCc1cccc(C=O)n1 ; COCc1cccc(CN2C(=O)c3cccc(c3)S(=O)(=O)Nc3nc(cc(-c4c(C)cccc4C)n3)OC[C@H]2CC(C)(C)C)n1]

3-[[4-[(2R)-2-amino-4,4-dimethyl-pentoxy]-6-(2,6-dimethylphenyl)pyrimidin-2-yl]sulfamoyl]benzoic acid (50 mg, 0.09754 mmol) was combined with 6-(methoxymethyl)pyridine-2-carbaldehyde (approximately 17.69 mg, 0.1170 mmol) in DCM (0.5 mL), and stirred for 15 minutes at room temperature. Sodium triacetoxyborohydride (approximately 20.67 mg, 0.09754 mmol) (1 equiv) was added, followed by additional sodium triacetoxyborohydride (approximately 62.01 mg, 0.2926 mmol) (3 equiv) after an additional 15 minutes. The reaction mixture was stirred at room temperature for an additional 2.5 hours, then quenched with 0.4 mL 1M HCl and diluted with 1:1 DMSO methanol until the reaction mixture became homogenous. The reaction mixture was then filtered and purified by reverse phase HPLC (1-70% ACN in water, HCl modifier, 15 min run) to give the reductive amination product upon drying. The product was combined with CDMT (approximately 11.99 mg, 0.06828 mmol) in DMF (1.5 mL), and N-methylmorpholine (approximately 44.39 mg, 48.25 μL, 0.4389 mmol) was added. The reaction was stirred at room temperature for 3 hours (Reaction 7 was allowed to stir for 16 hours out of convenience.) After this point the reaction was filtered and purified by reverse phase HPLC (1-99% ACN in water HCl modifier, 15 min run) to give upon drying the (11R)-6-(2,6-dimethylphenyl)-11-(2,2-dimethylpropyl)-12-[[6-(methoxymethyl)-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (6.6 mg, 10%). ESI-MS m/z calc. 629.2672, found 630.4 (M+1)⁺; Retention time: 1.69 minutes; LC method A.

### Example 107: Preparation of Compound 131

### Step 1: (11R)-6-(2,6-Dimethylphenyl)-11-(2,2-dimethylpropyl)-12-({6-[ethyl(propan-2-yl)amino]pyridin-2-yl}methyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 131)

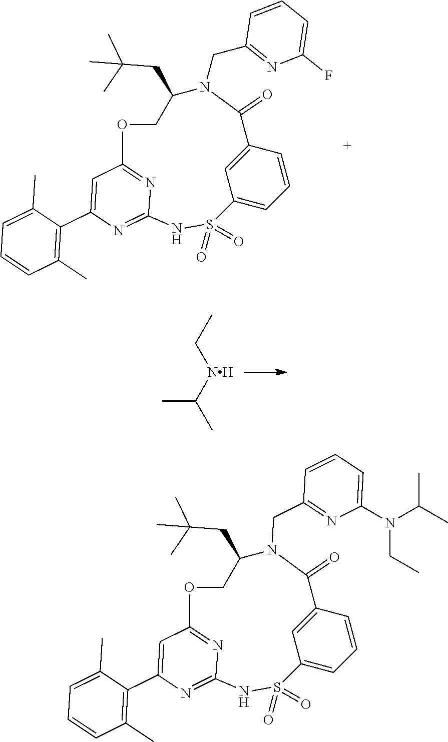

To a stirred solution of (11R)-6-(2,6-dimethylphenyl)-11-(2,2-dimethylpropyl)-12-[(6-fluoropyridin-2-yl)methyl]-9-oxa-2λ⁶-thia-3,5,12,19-tetraazatricyclo[12.3.1.14,8]nonadeca-1(17),4,6,8(19),14(18),15-hexaene-2,2,13-trione (14 mg, 0.02319 mmol) in anhydrous dimethyl sulfoxide (0.3 mL) were added N-ethylpropan-2-amine (25 mg, 0.2868 mmol) and potassium carbonate (25 mg, 0.1809 mmol), in that order, at room temperature. The contents were stirred at 130° C. in a closed vessel for 18 h (overnight) and at 160° C. for 18 h (overnight). The heterogeneous reaction mixture was allowed to cool to ambient temperature and purified by preparative reverse-phase HPLC [1-99% acetonitrile in water (containing 5 mM HCl as modifier) over 15 min] to furnish (11R)-6-(2,6-dimethylphenyl)-11-(2,2-dimethylpropyl)-12-({6-[ethyl(propan-2-yl)amino]pyridin-2-yl}methyl)-9-oxa-2λ⁶-thia-3,5,12,19-tetraazatricyclo[12.3.1.14,8]nonadeca-1(17),4,6,8(19),14(18),15-hexaene-2,2,13-trione (hydrochloride salt) (3 mg, 18%) as a white solid. ESI-MS m/z calc. 670.33014, found 671.5 (M+1)⁺; Retention time: 1.61 minutes; LC method A. ¹H NMR (400 MHz, DMSO-d₆) δ 8.70 (s, 1H), 7.97 (dd, J=5.4, 3.0 Hz, 1H), 7.79-7.65 (m, 2H), 7.56 (s, 1H), 7.26 (t, J=7.6 Hz, 1H), 7.12 (d, J=7.7 Hz, 2H), 6.67 (s, 2H), 6.44 (s, 1H), 5.34 (dd, J=10.7, 4.4 Hz, 1H), 4.81 (s, 1H), 4.74 (d, J=15.7 Hz, 1H), 4.49 (s, 1H), 4.28 (t, J=11.2 Hz, 1H), 4.14-4.03 (m, 1H), 2.57-2.51 (m, 2H), 2.01 (s, 6H), 1.76 (dd, J=15.3, 9.0 Hz, 1H), 1.39 (d, J=15.1 Hz, 1H), 1.22 (d, J=6.8 Hz, 3H), 1.17 (d, J=7.0 Hz, 3H), 1.15 (t, J=7.1 Hz, 3H), 0.51 (s, 9H).

### Example 108: Preparation of Compound 132

### Step 1: (11R)-6-(2,6-Dimethylphenyl)-11-(2,2-dimethylpropyl)-12-({6-[(2R)-2-methylpyrrolidin-1-yl]pyridin-2-yl}methyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 132)

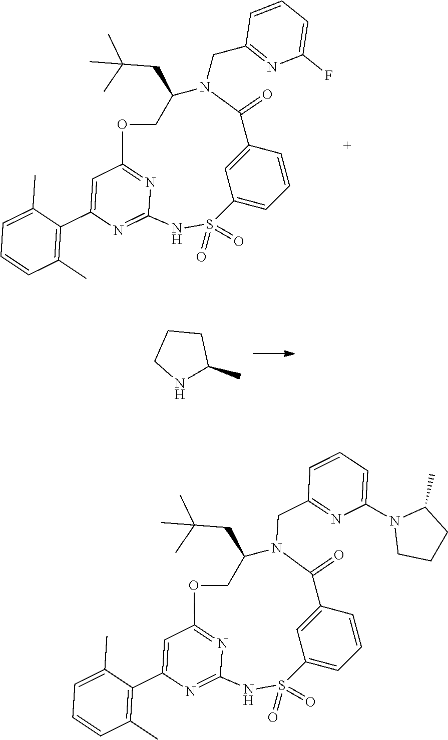

To a stirred solution of (11R)-6-(2,6-dimethylphenyl)-11-(2,2-dimethylpropyl)-12-[(6-fluoropyridin-2-yl)methyl]-9-oxa-2λ⁶-thia-3, 5,12,19-tetraazatricyclo[12.3.1.14,8]nonadeca-1(17),4,6,8(19),14(18),15-hexaene-2,2,13-trione (14 mg, 0.02319 mmol) in anhydrous dimethyl sulfoxide (0.3 mL) were added (2R)-2-methylpyrrolidine (hydrochloride salt) (15 mg, 0.1233 mmol) and potassium carbonate (35 mg, 0.2532 mmol), in that order, at room temperature. The contents were stirred at 130° C. in a closed vessel for 18 h (overnight), then stirred at 160° C. for 18 h (overnight). The heterogeneous reaction mixture was allowed to cool to ambient temperature and purified by preparative reverse-phase HPLC [1-99% acetonitrile in water (containing 5 mM HCl as modifier) over 15 min] to furnish (11R)-6-(2,6-dimethylphenyl)-11-(2,2-dimethylpropyl)-12-({6-[(2R)-2-methylpyrrolidin-1-yl]pyridin-2-yl}methyl)-9-oxa-2λ⁶-thia-3,5,12,19-tetraazatricyclo[12.3.1.14,8]nonadeca-1(17),4,6,8(19),14(18),15-hexaene-2,2,13-trione (hydrochloride salt) (6 mg, 36%) as a white solid. ESI-MS m/z calc. 668.31445, found 669.4 (M+1)⁺; Retention time: 1.45 minutes; LC method A. ¹H NMR (400 MHz, DMSO-d₆) δ 8.73 (s, 1H), 7.96 (s, 1H), 7.69 (s, 2H), 7.49 (s, 1H), 7.26 (t, J 7.6 Hz, 1H), 7.12 (d, J 7.7 Hz, 2H), 6.62 (s, 1H), 6.43 (s, 2H), 5.47 (s, 1H), 4.71 (s, 1H), 4.41 (s, 1H), 4.34-4.16 (m, 2H), 4.11-3.96 (m, 1H), 3.67-3.51 (m, 2H), 2.15-1.87 (m, 9H), 1.75 (dd, J 15.3, 9.0 Hz, 1H), 1.71 (s, 1H), 1.41 (d, J 15.0 Hz, 1H), 1.20 (d, J 6.3 Hz, 3H), 0.54 (s, 9H). (sulfonamide N—H peak missing)

### Example 109: Preparation of Compound 133

### Step 1: 6-Bromo-N-(2,2-dimethylpropyl)-N-methyl-pyridin-2-amine

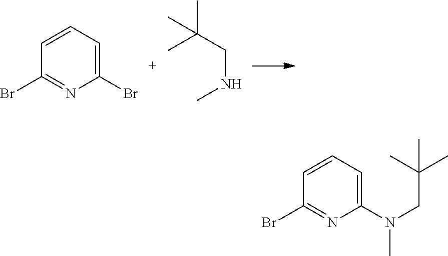

To a solution of N-methyl neopentylamine (hydrochloride salt) (1.3 g, 9.4442 mmol) in DMF (30 mL) was added 2,6-dibromopyridine (2 g, 8.4427 mmol) and potassium carbonate (234 mg, 1.6931 mmol) and the mixture was stirred for 48 h at 120-130° C. The reaction mixture was partitioned between water (100 mL) and ethyl acetate (150 mL). The aqueous layer was back-extracted with ethyl acetate (2×100 mL). The organic phase was washed with a 1:1 v/v mixture of brine and water (4×80 mL), dried over sodium sulfate and filtered and concentrated to dryness. The crude was purified by reverse phase chromatography (Cis, 80 g column) eluting with 10% to 100% methanol in water to give 6-bromo-N-(2,2-dimethylpropyl)-N-methyl-pyridin-2-amine (1.68 g, 77%) as a pale-yellow oil. ¹H NMR (400 MHz, CDCl₃) δ 7.22 (dd, J 8.3, 7.6 Hz, 1H), 6.65 (d, J 7.3 Hz, 1H), 6.41 (d, J 8.6 Hz, 1H), 3.37 (s, 2H), 3.07 (s, 3H), 0.97 (s, 9H). ESI-MS m/z calc. 256.05753, found 257.2 (M+1)⁺; Retention time: 2.15 minutes; LC method X.

### Step 2: 6-[2,2-Dimethylpropyl(methyl)amino]pyridine-2-carbaldehyde

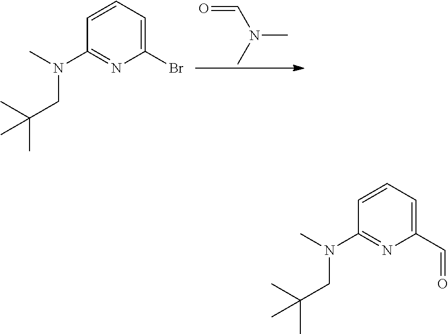

To a solution of 6-bromo-N-(2,2-dimethylpropyl)-N-methyl-pyridin-2-amine (1.68 g, 6.5326 mmol) in THE (13.6 mL) was dropwise added n-butyllithium solution in hexane (3.2 mL of 2.5 M, 8.0000 mmol) at −78° C. After stirring for 1 hour at this temperature, N,N-dimethylformamide (877.92 mg, 0.93 mL, 12.011 mmol) was slowly added and the reaction mixture was stirred for 1 hour at −78° C. The reaction mixture was warmed to room temperature and quenched with a saturated aqueous solution of ammonium chloride (10 mL) and extracted with ethyl acetate (3×20 mL). The organic phase was washed with brine (2×15 mL), dried over sodium sulfate, filtered and concentrated under reduced pressure. The crude was purified by flash chromatography on silica gel (SNAP 100 g) using a gradient of 0% to 10% of ethyl acetate in heptanes to give 6-[2,2-dimethylpropyl(methyl)amino]pyridine-2-carbaldehyde (860 mg, 62%) as a yellow oil. ¹H NMR (400 MHz, CDCl₃) δ 9.89 (d, J 0.7 Hz, 1H), 7.56 (t, J 7.6 Hz, 1H), 7.20 (d, J 7.1 Hz, 1H), 6.75 (d, J 8.6 Hz, 1H), 3.50 (s, 2H), 3.16 (s, 3H), 1.00 (s, 9H). ESI-MS m/z calc. 206.1419, found 207.2 (M+1)⁺; Retention time: 4.6 minutes; LC method Y.

### Step 3: 3-[[4-(2,6-Dimethylphenyl)-6-[(2R)-2-[[6-[2,2-dimethylpropyl(methyl)amino]-2-pyridyl]methylamino]-4,4-dimethyl-pentoxy]pyrimidin-2-yl]sulfamoyl]benzoic acid

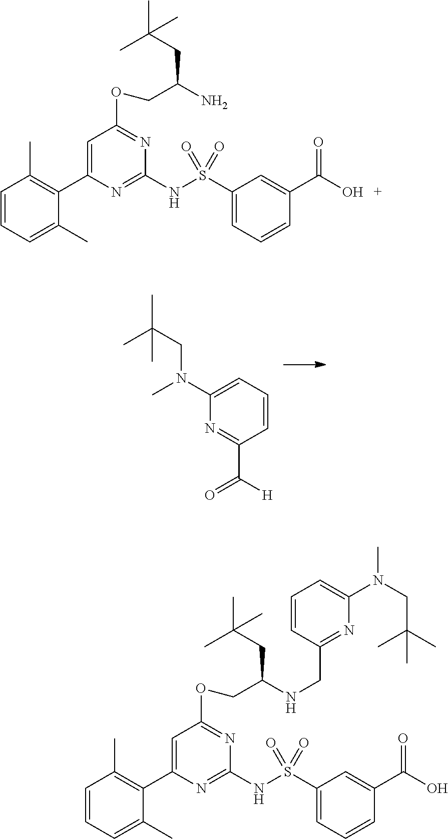

To a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (150 mg, 0.2571 mmol) and 6-[2,2-dimethylpropyl(methyl)amino]pyridine-2-carbaldehyde (160 mg, 0.5864 mmol) in anhydrous dichloromethane (2.25 mL) were added acetic acid (glacial) (33.792 mg, 32 L, 0.5627 mmol), triethylamine (72.600 mg, 0.1 mL, 0.7175 mmol) and sodium triacetoxyborohydride (272 mg, 1.2834 mmol), in that order. The vial was purged with nitrogen briefly and capped and allowed to stir at ambient temperature for 40 min under nitrogen. Then the mixture was poured over cold hydrochloric acid (20 mL of 1.0 M) and extracted with dichloromethane (3×30 mL). The combined organics were washed with brine (2×10 mL), dried over sodium sulfate, filtered and concentrated under reduced pressure. The crude was purified by reverse phase chromatography (Cis, 30 g) using a gradient of 10% to 100% of acetonitrile in acidic water (containing 0.1% w/w of hydrochloric acid) to give crude 3-[[4-(2,6-dimethylphenyl)-6-[(2R)-2-[[6-[2,2-dimethylpropyl(methyl)amino]-2-pyridyl]methylamino]-4,4-dimethyl-pentoxy]pyrimidin-2-yl]sulfamoyl]benzoic acid (hydrochloride salt) (93 mg, 49%). Presence of at least one non-identified impurity. No purity given. The crude product was used as is for the next step. ESI-MS m/z calc. 702.3563, found 703.2 (M+1)⁺; Retention time: 1.7 minutes; LC method X.

### Step 4: (11R)-6-(2,6-Dimethylphenyl)-11-(2,2-dimethylpropyl)-12-[[6-[2,2-dimethylpropyl(methyl)amino]-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 133)

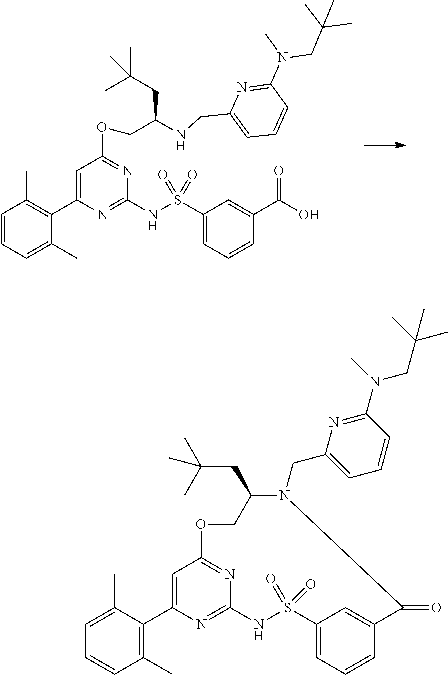 [SMILES: Cc1cccc(C)c1-c1cc(OC[C@@H](CC(C)(C)C)NCc2cccc(N(C)CC(C)(C)C)n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cccc(N(C)CC(C)(C)C)n1)[C@H](CC(C)(C)C)CO2]

To a stirring solution of N-methylmorpholine (66.240 mg, 0.072 mL, 0.6549 mmol) in DMF (7.5 mL) at 0° C. was added 2-chloro-4,6-dimethoxy-1,3,5-triazine (41 mg, 0.2335 mmol) followed by 3-[[4-(2,6-dimethylphenyl)-6-[(2R)-2-[[6-[2,2-dimethylpropyl(methyl)amino]-2-pyridyl]methylamino]-4,4-dimethyl-pentoxy]pyrimidin-2-yl]sulfamoyl]benzoic acid (hydrochloride salt) (90 mg, 0.1134 mmol) in DMF (4 mL). After 5 minutes the reaction mixture was warmed to room temperature and stirred for 3 days (over weekend). The solvent was removed under reduced pressure at 50° C. The crude was purified by reverse phase chromatography (Cis 30 g column) using a gradient of 5% to 100% acetonitrile in acidic water (0.1% v/v of formic acid in water). The fractions containing the product were evaporated and then lyophilized to give (11R)-6-(2,6-dimethylphenyl)-11-(2,2-dimethylpropyl)-12-[[6-[2,2-dimethylpropyl(methyl)amino]-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (37 mg, 46%) as a pale pink solid. ESI-MS m/z calc. 684.34576, found 685.4 (M+1)⁺; Retention time: 4.17 minutes; LC method Y. ¹H NMR (400 MHz, DMSO-d₆) δ 13.05 (br s, 1H), 8.68 (br s, 1H), 8.03-7.88 (m, 1H), 7.78-7.62 (m, 2H), 7.44 (dd, J 8.3, 7.3 Hz, 1H), 7.31-7.21 (m, 1H), 7.18-7.07 (m, 2H), 6.58 (dd, J 12.7, 7.8 Hz, 2H), 6.52-6.32 (m, 1H), 5.41-5.30 (m, 1H), 4.74 (d, J 15.7 Hz, 1H), 4.34 (d, J 15.4 Hz, 1H), 4.20 (t, J=11.0 Hz, 1H), 4.10-4.00 (m, 1H), 3.41 (s, 2H), 3.12 (s, 3H), 2.25-1.84 (m, 6H), 1.78 (dd, J 14.7, 8.8 Hz, 1H), 1.43-1.34 (m, 1H), 0.91 (s, 9H), 0.55 (s, 9H).

### Example 110: Preparation of Compound 134

### Step 1: (11R)-12-[(5-tert-Butyl-1H-pyrazol-3-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 134)

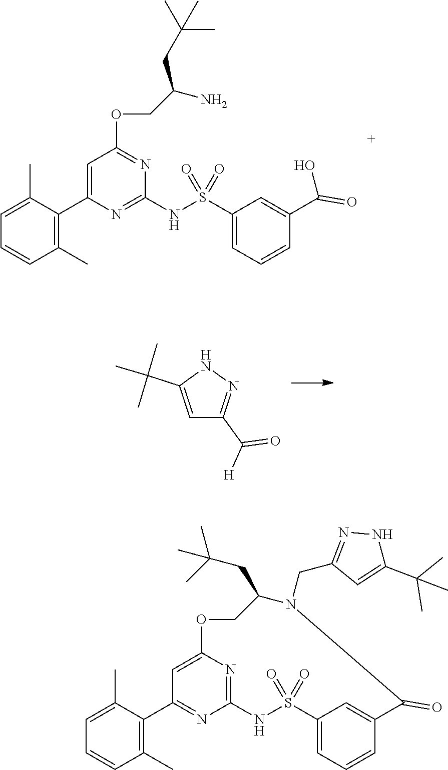 [SMILES: Cc1cccc(C)c1-c1cc(OC[C@H](N)CC(C)(C)C)nc(NS(=O)(=O)c2cccc(C(=O)O)c2)n1 ; "+" ; CC(C)(C)c1cc(C=O)n[nH]1 ; Cc1cccc(C)c1-c1cc2nc(n1)NS(=O)(=O)c1cccc(c1)C(=O)N(Cc1cc(C(C)(C)C)[nH]n1)[C@H](CC(C)(C)C)CO2]

3-[[4-[(2R)-2-Amino-4,4-dimethyl-pentoxy]-6-(2,6-dimethylphenyl)pyrimidin-2-yl]sulfamoyl]benzoic acid (50 mg, 0.09754 mmol) was combined with 5-tert-butyl-1H-pyrazole-3-carbaldehyde (approximately 17.81 mg, 0.1170 mmol) in DCM (0.3 mL), and stirred for 15 minutes at room temperature. Sodium triacetoxyborohydride (approximately 20.67 mg, 0.09754 mmol) (1 equiv) was added, followed by additional sodium triacetoxyborohydride (approximately 62.01 mg, 0.2926 mmol) (3 equiv) after an additional 15 minutes. The reaction mixture was stirred at room temperature for an additional 2.5 hours, then quenched with 0.4 mL 1M HCl and diluted with 1:1 DMSO methanol until the reaction mixture became homogenous. The reaction mixture was then filtered and purified by reverse phase HPLC (1-70% ACN in water, HCl modifier, 15 min run) to give the reductive amination product upon drying. The product from step 1 was combined with CDMT (approximately 11.13 mg, 0.06340 mmol) in DMF (1.5 mL), and N-methylmorpholine (approximately 34.53 mg, 37.53 μL, 0.3414 mmol) was added. The reaction was stirred at room temperature 3 hours. The reaction mixture was then filtered and purified by reverse phase HPLC (1-99% ACN in water, HCl modifier, 15 min run) to give upon drying (11R)-12-[(5-tert-butyl-1H-pyrazol-3-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (12.4 mg, 20%). ESI-MS m/z calc. 630.2988, found 631.3 (M+1)⁺; Retention time: 1.77 minutes; LC method A.

### Example 111: Preparation of Compound 135

### Step 1: 3-[[4-(2,6-Dimethylphenyl)-6-[(2R)-4,4-dimethyl-2-(pyridazin-3-ylmethylamino)pentoxy]pyrimidin-2-yl]sulfamoyl]benzoic acid

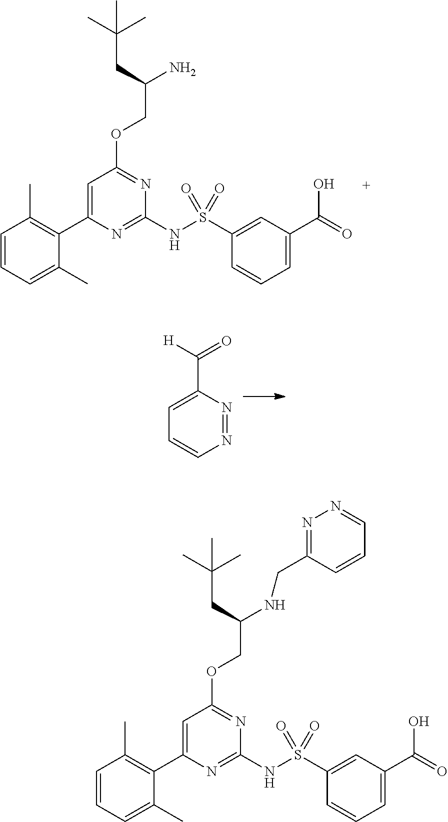 [SMILES: Cc1cccc(C)c1-c1cc(OC[C@H](N)CC(C)(C)C)nc(NS(=O)(=O)c2cccc(C(=O)O)c2)n1 ; O=Cc1cccnn1 ; Cc1cccc(C)c1-c1cc(OC[C@@H](CC(C)(C)C)NCc2cccnn2)nc(NS(=O)(=O)c2cccc(C(=O)O)c2)n1]

3-[[4-[(2R)-2-Amino-4,4-dimethyl-pentoxy]-6-(2,6-dimethylphenyl)pyrimidin-2-yl]sulfamoyl]benzoic acid (hydrochloride salt) (70.1 mg, 0.1277 mmol), pyridazine-3-carbaldehyde (19.7 mg, 0.1822 mmol), triethylamine (23 μL, 0.1650 mmol), and acetic acid (11 μL, 0.1934 mmol) were combined in DCM (500 μL) and stirred at room temperature for 5 min. At this point, everything was in solution. Sodium triacetoxyborohydride (86 mg, 0.4058 mmol) was added and the reaction was stirred an additional 1.5 h at room temperature. The reaction mixture was quenched with methanol, and 1M HCl. The solvents were evaporated and the resulting material was purified by reverse-phase HPLC utilizing a gradient of 1-99% acetonitrile in 5 mM aqueous HCl to yield 3-[[4-(2,6-dimethylphenyl)-6-[(2R)-4,4-dimethyl-2-(pyridazin-3-ylmethylamino)pentoxy]pyrimidin-2-yl]sulfamoyl]benzoic acid (hydrochloride salt) (44.3 mg, 54%). ESI-MS m/z calc. 604.24677, found 605.6 (M+1)⁺; Retention time: 0.47 minutes; LC method D.

### Step 2: (11R)-6-(2,6-Dimethylphenyl)-11-(2,2-dimethylpropyl)-12-[(pyridazin-3-yl)methyl]-9-oxa-2)⁶-thia-3,5,12,19-tetraazatricyclo[12.3.1.14,8]nonadeca-1(18),4,6,8(19),14,16-hexaene-2,2,13-trione (Compound 135)

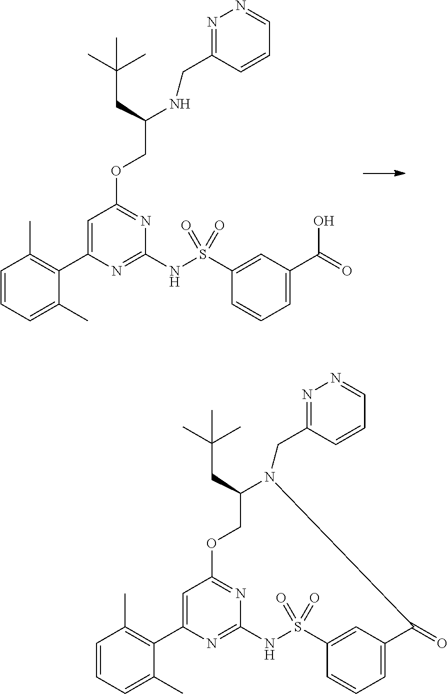 [SMILES: Cc1cccc(C)c1-c1cc(OC[C@@H](CC(C)(C)C)NCc2cccnn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cccnn1)[C@H](CC(C)(C)C)CO2]

3-[[4-(2,6-Dimethylphenyl)-6-[(2R)-4,4-dimethyl-2-(pyridazin-3-ylmethylamino)pentoxy]pyrimidin-2-yl]sulfamoyl]benzoic acid (hydrochloride salt) (26.1 mg, 0.04071 mmol), COMU (26.9 mg, 0.06281 mmol), and DIEA (35 μL, 0.2009 mmol) were combined in DMF (1 mL) and stirred at room temperature for 16 h. The reaction mixture was filtered and purified by reverse phase HPLC utilizing a gradient of 1-99% acetonitrile in 5 mM aqueous HCl to yield (11R)-6-(2,6-dimethylphenyl)-11-(2,2-dimethylpropyl)-12-[(pyridazin-3-yl)methyl]-9-oxa-2λ⁶-thia-3,5,12,19-tetraazatricyclo[12.3.1.14,8]nonadeca-1(18),4,6,8(19),14,16-hexaene-2,2,13-trione (5.4 mg, 22%) as a light yellow solid. ESI-MS m z calc. 586.2362, found 587.2 (M+1)⁺; Retention time: 1.49 minutes; LC method A.

### Example 112: Preparation of Compound 136

### Step 1: 3-[[4-[(2R)-2-[(2-aminopyrimidin-5-yl)methylamino]-4,4-dimethyl-pentoxy]-6-(2,6-dimethylphenyl)pyrimidin-2-yl]sulfamoyl]benzoic acid

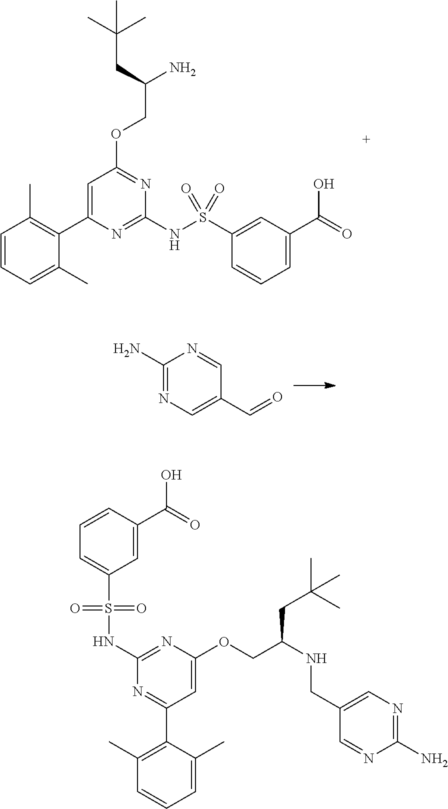 [SMILES: Cc1cccc(C)c1-c1cc(OC[C@H](N)CC(C)(C)C)nc(NS(=O)(=O)c2cccc(C(=O)O)c2)n1 ; Nc1ncc(C=O)cn1 ; Cc1cccc(C)c1-c1cc(OC[C@@H](CC(C)(C)C)NCc2cnc(N)nc2)nc(NS(=O)(=O)c2cccc(C(=O)O)c2)n1]

3-[[4-[(2R)-2-Amino-4,4-dimethyl-pentoxy]-6-(2,6-dimethylphenyl)pyrimidin-2-yl]sulfamoyl]benzoic acid (hydrochloride salt) (100 mg, 0.1821 mmol), 2-aminopyrimidine-5-carbaldehyde (approximately 33.63 mg, 0.2732 mmol), and sodium triacetoxyborohydride (154.7 mg, 0.73 mmol) were combined in dichloromethane (0.5 mL) and stirred at room temperature for 2 h. The reaction mixture was partitioned between ethyl acetate and a 1M HCl solution. The organics were separated, washed with brine, dried over sodium sulfate and evaporated. The crude material was purified by reverse phase HPLC utilizing a gradient of 1-99% acetonitrile in 5 mM aqueous HCl to yield 3-[[4-[(2R)-2-[(2-aminopyrimidin-5-yl)methylamino]-4,4-dimethyl-pentoxy]-6-(2,6-dimethylphenyl)pyrimidin-2-yl]sulfamoyl]benzoic acid (hydrochloride salt) (51.2 mg, 43%). ESI-MS m/z calc. 619.2577, found 620.6 (M+1)⁺; Retention time: 0.46 minutes; LC method D.

### Step 2: (11R)-12-[(2-Aminopyrimidin-5-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (Compound 136)

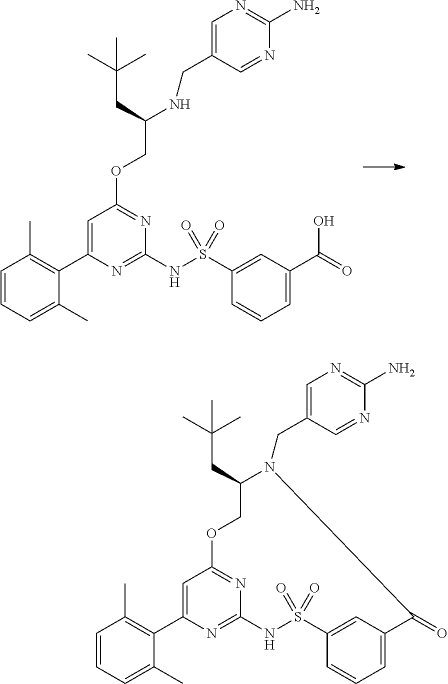 [SMILES: Cc1cccc(C)c1-c1cc(OC[C@@H](CC(C)(C)C)NCc2cnc(N)nc2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnc(N)nc1)[C@H](CC(C)(C)C)CO2]

3-[[4-[(2R)-2-[(2-Aminopyrimidin-5-yl)methylamino]-4,4-dimethyl-pentoxy]-6-(2,6-dimethylphenyl)pyrimidin-2-yl]sulfamoyl]benzoic acid (hydrochloride salt) (19.2 mg, 0.02926 mmol), DIEA (26 μL, 0.1493 mmol), and COMU (21.7 mg, 0.05067 mmol) were combined in DMF (1 mL) and stirred at room temperature for 5 h. The reaction mixture was filtered and purified by reverse phase HPLC utilizing a gradient of 1-99% acetonitrile in 5 mM aqueous HCl to yield (11R)-12-[(2-aminopyrimidin-5-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(18),4,6,8(19),14,16-hexaene-2,2,13-trione (5.8 mg, 33%) as a white solid. ESI-MS m/z calc. 601.24713, found 602.7 (M+1)⁺; Retention time: 1.33 minutes; LC method A.

### Example 113: Preparation of Compound 137

### Step 1: 3-[[4-[(2R)-2-[(3-Cyclopropylisoxazol-5-yl)methylamino]-4,4-dimethyl-pentoxy]-6-(2,6-dimethylphenyl)pyrimidin-2-yl]sulfamoyl]benzoic acid

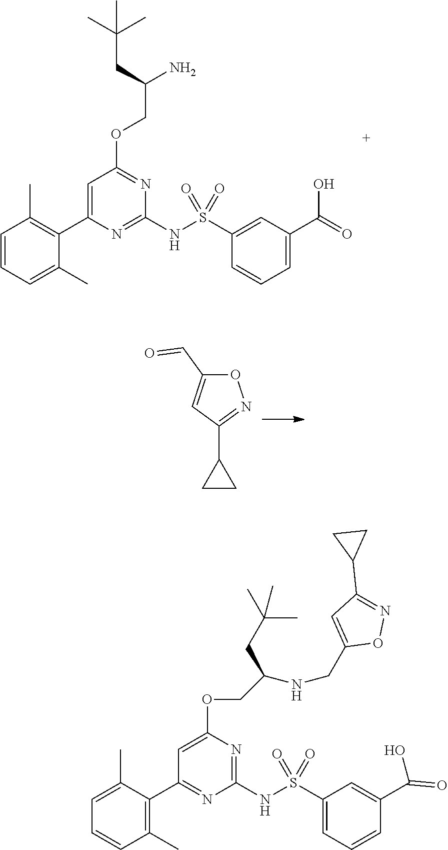 [SMILES: Cc1cccc(C)c1-c1cc(OC[C@H](N)CC(C)(C)C)nc(NS(=O)(=O)c2cccc(C(=O)O)c2)n1 ; O=Cc1cc(C2CC2)no1 ; Cc1cccc(C)c1-c1cc(OC[C@@H](CC(C)(C)C)NCc2cc(C3CC3)no2)nc(NS(=O)(=O)c2cccc(C(=O)O)c2)n1]

1:3-[[4-[(2R)-2-Amino-4,4-dimethyl-pentoxy]-6-(2,6-dimethylphenyl)pyrimidin-2-yl]sulfamoyl]benzoic acid (hydrochloride salt) (49.5 mg, 0.09015 mmol), 3-cyclopropylisoxazole-5-carbaldehyde (15.2 mg, 0.1108 mmol), and sodium triacetoxyborohydride (45.7 mg, 0.2156 mmol) were combined in dichloromethane (600 μL) and stirred for 1 h. The reaction was quenched with aqueous HCl (45 μL of 6 M, 0.2700 mmol) and diluted with methanol (0.5 mL). The solution was filtered and purified by reverse-phase HPLC utilizing a gradient of 1-99% acetonitrile in 5 mM aqueous HCl to yield 3-[[4-[(2R)-2-[(3-cyclopropylisoxazol-5-yl)methylamino]-4,4-dimethyl-pentoxy]-6-(2,6-dimethylphenyl)pyrimidin-2-yl]sulfamoyl]benzoic acid (hydrochloride salt) (10.3 mg, 17%) as a white solid. ESI-MS m/z calc. 633.2621, found 634.5 (M+1)⁺; Retention time: 0.52 minutes; LC method D.

### Step 2: (11R)-12-[(3-Cyclopropyl-1,2-oxazol-5-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (Compound 137)

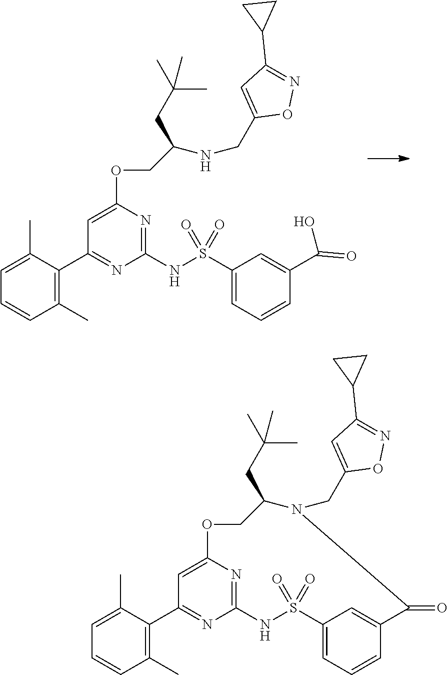 [SMILES: Cc1cccc(C)c1-c1cc(OC[C@@H](CC(C)(C)C)NCc2cc(C3CC3)no2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cc(C3CC3)no1)[C@H](CC(C)(C)C)CO2]

3-[[4-[(2R)-2-[(3-Cyclopropylisoxazol-5-yl)methylamino]-4,4-dimethyl-pentoxy]-6-(2,6-dimethylphenyl)pyrimidin-2-yl]sulfamoyl]benzoic acid (hydrochloride salt) (10.3 mg, 0.01537 mmol), HATU (9.7 mg, 0.02551 mmol), and triethylamine (13 μL, 0.09327 mmol) were combined in DMF (1 mL) and stirred at room temperature for 2 h. The reaction mixture was purified by reverse-phase HPLC utilizing a gradient of 1-99% acetonitrile in 5 mM aqueous HCl to yield (11R)-12-[(3-cyclopropyl-1,2-oxazol-5-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (4.2 mg, 44%). ESI-MS m/z calc. 615.2515, found 616.5 (M+1)⁺; Retention time: 1.85 minutes; LC method A.

### Example 114: Preparation of Compound 138

### Step 1: 3-[[4-[(2R)-2-(3,3-Dimethylbutylamino)-4,4-dimethyl-pentoxy]-6-(2,6-dimethylphenyl)pyrimidin-2-yl)sulfamoyl]benzoic acid

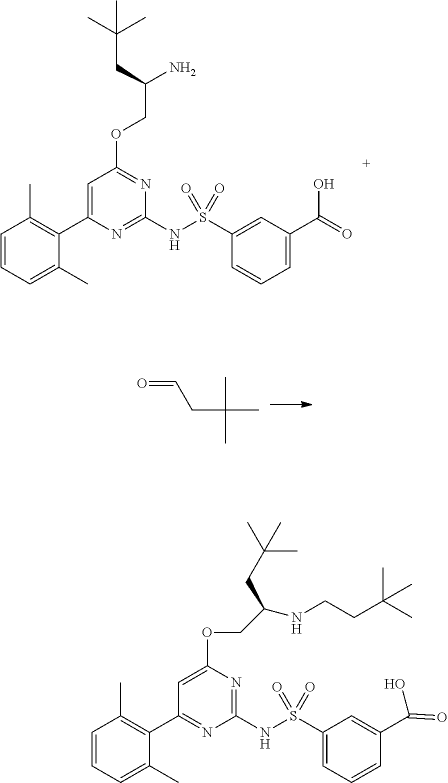

3-[[4-[(2R)-2-Amino-4,4-dimethyl-pentoxy]-6-(2,6-dimethylphenyl)pyrimidin-2-yl]sulfamoyl]benzoic acid (hydrochloride salt) (40 mg, 0.07285 mmol) was combined with 3,3-dimethylbutanal (approximately 14.59 mg, 18.28 μL, 0.1457 mmol) and acetic acid (approximately 35.00 mg, 33.14 μL, 0.5828 mmol) in DCE (0.4 mL) and stirred at room temperature for 20 minutes, at which point sodium cyanoborohydride (approximately 13.74 mg, 0.2186 mmol) was added. At this point the reactions were quenched with 2 drops 1 M HCl, concentrated then dissolved in 1:1 DMSO/methanol, filtered and purified by reverse phase HPLC (1-70% ACN, HCl modifier) to give the corresponding 3-[[4-[(2R)-2-(3,3-dimethylbutylamino)-4,4-dimethyl-pentoxy]-6-(2,6-dimethylphenyl)pyrimidin-2-yl]sulfamoyl]benzoic acid (hydrochloride salt) (5 mg, 12%). ESI-MS m/z calc. 596.3032, found 597.5 (M+1)⁺; Retention time: 0.54 minutes; LC method D.

### Step 2: (11R)-12-(3,3-Dimethylbutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 138)

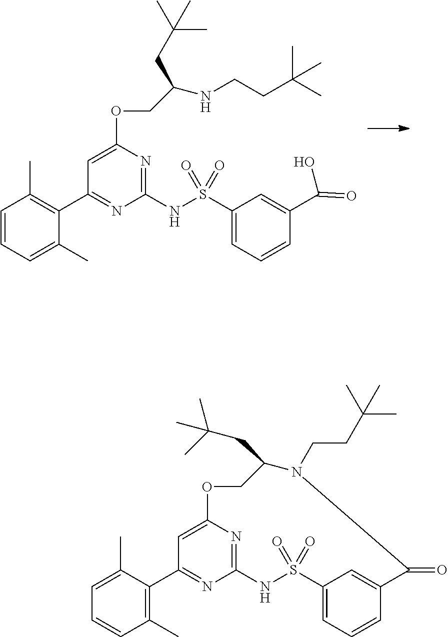 [SMILES: Cc1cccc(C)c1-c1cc(OC[C@@H](CC(C)(C)C)NCCC(C)(C)C)nc(NS(=O)(=O)c2cccc(C(=O)O)c2)n1 ; Cc1cccc(C)c1-c1cc2nc(n1)NS(=O)(=O)c1cccc(c1)C(=O)N(CCC(C)(C)C)[C@H](CC(C)(C)C)CO2]

The 3-[[4-[(2R)-2-(3,3-dimethylbutylamino)-4,4-dimethyl-pentoxy]-6-(2,6-dimethylphenyl)pyrimidin-2-yl]sulfamoyl]benzoic acid (hydrochloride salt) (5 mg, 0.008378 mmol) was combined with HATU (approximately 4.141 mg, 0.01089 mmol) in DMF (1 mL), and DIPEA (approximately 5.414 mg, 7.296 μL, 0.04189 mmol) was added. The reaction was stirred at room temperature for 1-2 hours, then filtered and purified by reverse phase HPLC (1-99% ACN in water, HCl modifier) to give the (11R)-12-(3,3-dimethylbutyl)-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.9 mg, 39%) products after drying. ESI-MS m/z calc. 578.29266, found 579.5 (M+1)⁺; Retention time: 2.13 minutes; LC method A.

### Example 115: Preparation of Compound 139

### Step 1: 3-[[4-(2,6-dimethylphenyl)-6-[(2R)-4,4-dimethyl-2-(pyrazolo[1,5-a]pyridin-3-ylmethylamino)pentoxy]pyrimidin-2-yl]sulfamoyl]benzoic acid

3-[[4-(2,6-dimethylphenyl)-6-[(2R)-4,4-dimethyl-2-(pyrazolo[1,5-a]pyridin-5-ylmethylamino)pentoxy]pyrimidin-2-yl]sulfamoyl]benzoic acid

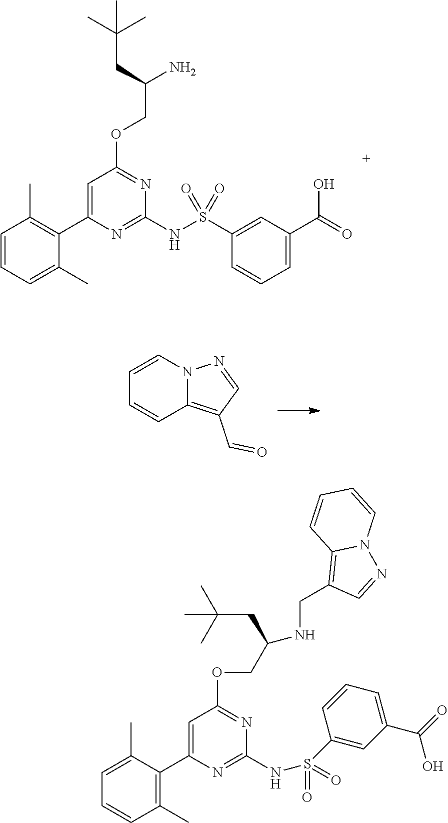 [SMILES: Cc1cccc(C)c1-c1cc(OC[C@H](N)CC(C)(C)C)nc(NS(=O)(=O)c2cccc(C(=O)O)c2)n1 ; O=Cc1cnn2ccccc12 ; Cc1cccc(C)c1-c1cc(OC[C@@H](CC(C)(C)C)NCc2cnn3ccccc23)nc(NS(=O)(=O)c2cccc(C(=O)O)c2)n1]

In a 4 mL vial, to a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (75 mg, 0.1366 mmol) and pyrazolo[1,5-a]pyridine-3-carbaldehyde (20 mg, 0.1368 mmol) in anhydrous dichloromethane (1 mL) were added glacial acetic acid (10 μL, 0.1758 mmol), sodium triacetoxyborohydride (125 mg, 0.5898 mmol) and DIEA (100 μL, 0.5741 mmol), in that order. The vial was purged with nitrogen briefly, capped and allowed to stir at ambient temperature for 2 h. Then methanol (0.3 mL) and water (0.2 mL) were added to the reaction and the volatiles were removed under reduced pressure and the residue was taken up in DMSO (1.5 mL), micro-filtered, and purified from reverse-phase HPLC (1-99% acetonitrile in water over 15 min, HCl as modifier) to furnish 3-[[4-(2,6-dimethylphenyl)-6-[(2R)-4,4-dimethyl-2-(pyrazolo[1,5-a]pyridin-3-ylmethylamino)pentoxy]pyrimidin-2-yl]sulfamoyl]benzoic acid (hydrochloride salt) (42 mg, 45%) as white solid. ESI-MS m/z calc. 642.26245, found 643.3 (M+1)⁺; Retention time: 1.18 minutes; LC method A. ¹H NMR (400 MHz, DMSO-d₆) δ 13.41 (s, 1H), 9.10 (s, 1H), 9.01 (s, 1H), 8.71 (d, J 7.0 Hz, 1H), 8.46 (t, J 1.8 Hz, 1H), 8.17 (s, 1H), 8.13 (t, J 6.8 Hz, 2H), 7.94-7.80 (m, 1H), 7.68 (t, J 7.8 Hz, 1H), 7.39-7.29 (m, 1H), 7.25 (d, J 7.6 Hz, 1H), 7.13 (d, J 7.6 Hz, 2H), 6.96 (td, J 6.8, 1.3 Hz, 1H), 6.37 (s, 1H), 4.46 (s, 3H), 4.28 (s, 1H), 3.60 (s, 1H), 2.02 (s, 6H), 1.80-1.62 (m, 2H), 0.91 (s, 9H). ESI-MS m/z calc. 642.26245, found 643.3 (M+1)⁺; Retention time: 1.17 minutes; LC method A.

### Step 2: (11R)-6-(2,6-Dimethylphenyl)-11-(2,2-dimethylpropyl)-12-({pyrazolo[1,5-a]pyridin-3-yl}methyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 139)

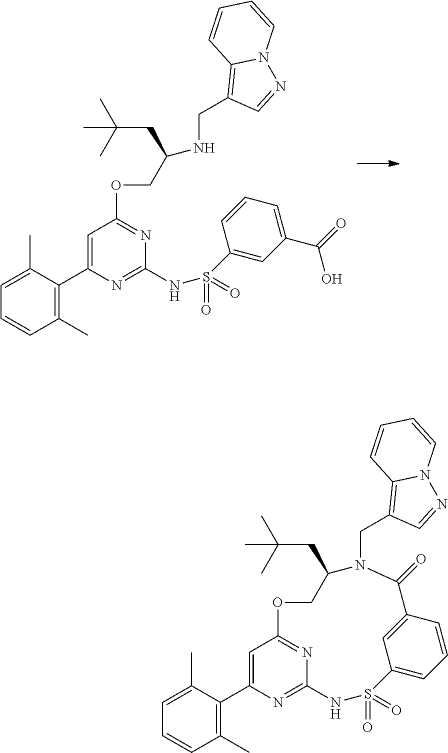 [SMILES: Cc1cccc(C)c1-c1cc(OC[C@@H](CC(C)(C)C)NCc2cnn3ccccc23)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cnn3ccccc13)[C@H](CC(C)(C)C)CO2]

In a 4 mL vial, to a stirred solution of 3-[[4-(2,6-dimethylphenyl)-6-[(2R)-4,4-dimethyl-2-(pyrazolo[1,5-a]pyridin-3-ylmethylamino)pentoxy]pyrimidin-2-yl]sulfamoyl]benzoic acid (hydrochloride salt) (40 mg, 0.05889 mmol) in anhydrous DMF (2 mL) were added [dimethylamino(triazolo[4,5-b]pyridin-3-yloxy)methylene]-dimethyl-ammonium (Phosphorus Hexafluoride Ion) (45 mg, 0.1183 mmol) (HATU) and DIEA (60 μL, 0.3445 mmol), in that order, at ambient temperature under nitrogen. Stirring continued for 4 h, then the solution was micro-filtered, and purified by preparative reverse-phase HPLC (Cis column, 1-99% acetonitrile in water over 15 min, HCl as modifier) to furnish (11R)-6-(2,6-dimethylphenyl)-11-(2,2-dimethylpropyl)-12-({pyrazolo[1,5-a]pyridin-3-yl}methyl)-9-oxa-2λ⁶-thia-3,5,12,19-tetraazatricyclo[12.3.1.14,8]nonadeca-1(17),4,6,8(19),14(18),15-hexaene-2,2,13-trione (21 mg, 57%) as a white solid. ESI-MS m/z calc. 624.2519, found 625.3 (M+1)⁺; Retention time: 1.76 minutes; LC method A. ¹H NMR (400 MHz, DMSO-d₆) δ 12.98 (s, 1H), 8.65 (d, J 7.0 Hz, 1H), 8.37 (s, 1H), 8.15 (s, 1H), 8.01 (d, J 9.0 Hz, 1H), 7.93 (d, J 6.9 Hz, 1H), 7.70 (d, J 6.8 Hz, 2H), 7.32-7.21 (m, 2H), 7.12 (d, J 7.7 Hz, 2H), 6.89 (td, J 6.8, 1.3 Hz, 1H), 6.39 (s, 1H), 5.07-4.88 (m, 2H), 4.66 (d, J 15.2 Hz, 1H), 4.39 (t, J 11.1 Hz, 1H), 3.98-3.83 (m, 1H), 2.15-1.78 (m, 7H), 1.40 (d, J=15.0 Hz, 1H), 0.43 (s, 9H).

### Example 116: Preparation of Compound 140

### Step 1: 3-[[4-[(2R)-4,4-dimethyl-2-[(1-methyltriazol-4-yl)methylamino]pentoxy]-6-(2,6-dimethylphenyl)pyrimidin-2-yl]sulfamoyl]benzoic acid

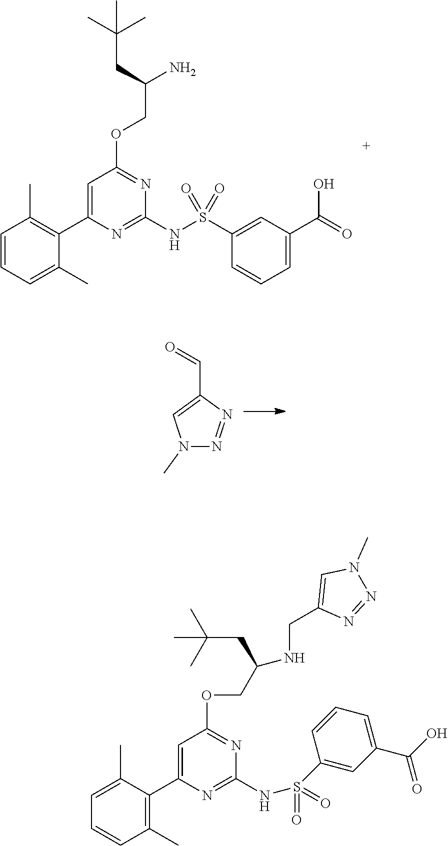

The compound was prepared in a manner analogous to that described above using 1-methyltriazole-4-carbaldehyde (13 mg, 0.1170 mmol) to give 3-[[4-[(2R)-4,4-dimethyl-2-[(1-methyltriazol-4-yl)methylamino]pentoxy]-6-(2,6-dimethylphenyl)pyrimidin-2-yl]sulfamoyl]benzoic acid (hydrochloride salt) (35 mg, 50%), obtained as white solid. It was used in the subsequent reaction. ESI-MS m/z calc. 607.2577, found 608.2 (M+1)⁺; Retention time: 1.09 minutes; LC method A.

### Step 2: (11R)-6-(2,6-Dimethylphenyl)-11-(2,2-dimethylpropyl)-12-[(1-methyltriazol-4-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 140)

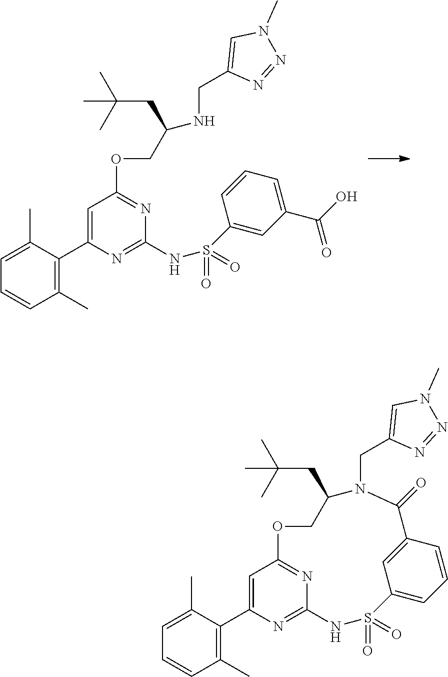 [SMILES: Cc1cccc(C)c1-c1cc(OC[C@@H](CC(C)(C)C)NCc2cn(C)nn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cn(C)nn1)[C@H](CC(C)(C)C)CO2]

In a 4 mL vial, to a stirred solution of 3-[[4-[(2R)-4,4-dimethyl-2-[(1-methyltriazol-4-yl)methylamino]pentoxy]-6-(2,6-dimethylphenyl)pyrimidin-2-yl]sulfamoyl]benzoic acid (hydrochloride salt) (30 mg, 0.04657 mmol) in anhydrous DMF (1.6 mL) was added [dimethylamino(triazolo[4,5-b]pyridin-3-yloxy)methylene]-dimethyl-ammonium (Phosphorus Hexafluoride Ion) (35 mg, 0.09205 mmol) (HATU), followed by addition of DIEA (50 μL, 0.2871 mmol) at ambient temperature. The vial was briefly purged with nitrogen and the capped vial was allowed to stir at ambient temperature for 15 h (overnight). Then DMSO (0.2 mL) and methanol (0.1 mL) were added, micro-filtered, and purified by preparative reverse-phase HPLC (1-99% acetonitrile in water over 15 min, HCl as modifier) and purified again using the above preparative HPLC conditions with a 20-80% gradient of acetonitrile over 30 min to give (11R)-6-(2,6-dimethylphenyl)-11-(2,2-dimethylpropyl)-12-[(1-methyltriazol-4-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (7 mg, 25%) as a white solid. ESI-MS m/z calc. 589.24713, found 590.3 (M+1)⁺; Retention time: 1.53 minutes; LC method A. ¹H NMR (400 MHz, DMSO-d₆) δ 8.42 (s, 1H), 8.04 (s, 1H), 7.94 (d, J 6.6 Hz, 1H), 7.79-7.57 (m, 2H), 7.26 (t, J 7.6 Hz, 1H), 7.13 (d, J 7.8 Hz, 2H), 6.44 (s, 1H), 5.12 (dd, J 11.0, 4.5 Hz, 1H), 4.77 (d, J 15.1 Hz, 1H), 4.52 (d, J 15.2 Hz, 1H), 4.37 (t, J 11.3 Hz, 1H), 4.04 (s, 3H), 3.98-3.86 (m, 1H), 2.19-1.75 (m, 7H), 1.39 (d, J 15.0 Hz, 1H), 0.48 (s, 9H).

### Example 117: Preparation of Compound 141

### Step 1: 3-[[4-[(2R)-2-[[(2R)-4-tert-butoxycarbonylmorpholin-2-yl]methylamino]-4,4-dimethyl-pentoxy]-6-(2,6-dimethylphenyl)pyrimidin-2-yl]sulfamoyl]benzoic acid

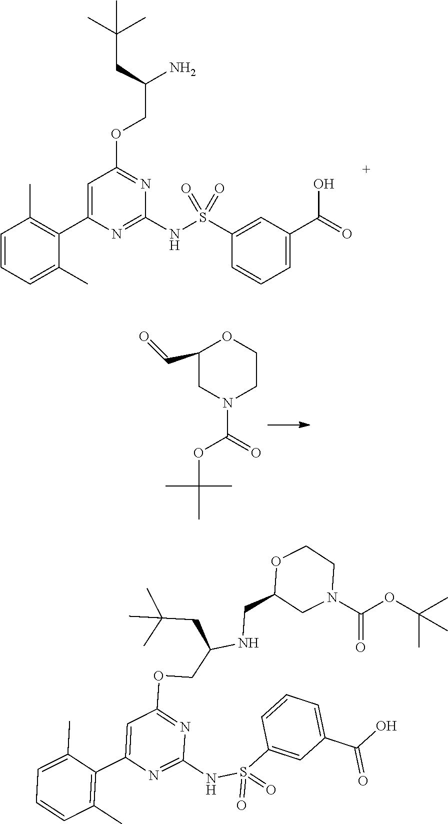 [SMILES: Cc1cccc(C)c1-c1cc(OC[C@H](N)CC(C)(C)C)nc(NS(=O)(=O)c2cccc(C(=O)O)c2)n1 ; CC(C)(C)OC(=O)N1CCO[C@H](C=O)C1 ; Cc1cccc(C)c1-c1cc(OC[C@@H](CC(C)(C)C)NC[C@@H]2CN(C(=O)OC(C)(C)C)CCO2)nc(NS(=O)(=O)c2cccc(C(=O)O)c2)n1]

To a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (300 mg, 0.5464 mmol) in anhydrous dichloromethane (2.3 mL) were added tert-butyl (2S)-2-formylmorpholine-4-carboxylate (119 mg, 0.5529 mmol), and glacial acetic acid (40 μL, 0.7034 mmol), in that order. The clear yellow solution was stirred at room temperature for 5 min. Then sodium triacetoxyborohydride (600 mg, 2.831 mmol) and DIEA (300 μL, 1.722 mmol) were added, in that order. After stirring for 1 h, methanol (1.5 mL) and water (1.0 mL) were added to the reaction and the volatiles were removed under reduced pressure and the residue was partitioned between ethyl acetate (20 mL) and brine (20 mL). and the aqueous layer was re-extracted with ethyl acetate (2×15 mL). The combined organics were concentrated under reduced pressure and the residue was purified by reverse-phase HPLC (1-99% acetonitrile in water over 15 min, 5 mM HCl as modifier). The desire fractions were combined and extracted with ethyl acetate (3×20 mL) and the combined organics were washed with brine (20 mL), dried over sodium sulfate, filtered, and concentrated under reduced pressure to furnish 3-[[4-[(2R)-2-[[(2R)-4-tert-butoxycarbonylmorpholin-2-yl]methylamino]-4,4-dimethyl-pentoxy]-6-(2,6-dimethylphenyl)pyrimidin-2-yl]sulfamoyl]benzoic acid (hydrochloride salt) (160 mg, 39%) as a white solid. It was used in the subsequent reaction. ESI-MS m/z calc. 711.3302, found 712.4 (M+1)⁺; Retention time: 1.34 minutes; LC method A. ¹H NMR (400 MHz, DMSO-d₆) δ 13.42 (s, 1H), 8.87 (s, 2H), 8.45 (d, J 2.1 Hz, 1H), 8.12 (d, J 8.9 Hz, 2H), 7.69 (t, J 7.8 Hz, 1H), 7.26 (s, 1H), 7.13 (d, J 7.6 Hz, 2H), 6.36 (s, 1H), 4.43 (s, 1H), 4.20 (s, 1H), 3.98-3.80 (m, 2H), 3.73 (d, J 12.2 Hz, 2H), 3.54 (s, 1H), 3.47-3.36 (m, 2H), 2.97 (t, J 11.1 Hz, 1H), 2.87 (s, 1H), 2.62 (s, 1H), 2.02 (s, 6H), 1.71-1.52 (m, 2H), 1.38 (s, 9H), 0.93 (s, 9H).

### Step 2: tert-Butyl (2R)-2-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]morpholine-4-carboxylate (Compound 141)

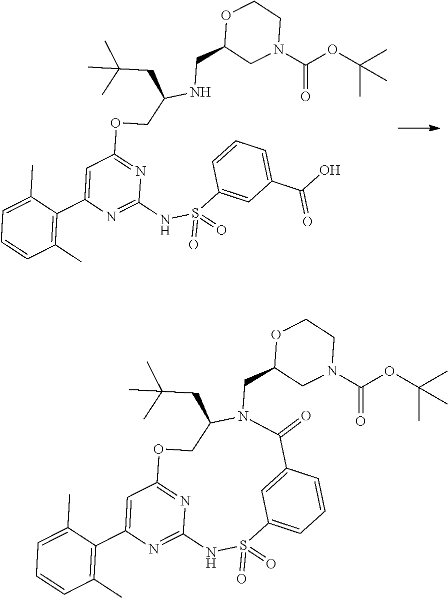 [SMILES: Cc1cccc(C)c1-c1cc(OC[C@@H](CC(C)(C)C)NC[C@@H]2CN(C(=O)OC(C)(C)C)CCO2)nc(NS(=O)(=O)c2cccc(C(=O)O)c2)n1 ; Cc1cccc(C)c1-c1cc2nc(n1)NS(=O)(=O)c1cccc(c1)C(=O)N(C[C@@H]1CN(C(=O)OC(C)(C)C)CCO1)[C@H](CC(C)(C)C)CO2]

To a stirred solution of 3-[[4-[(2R)-2-[[(2R)-4-tert-butoxycarbonylmorpholin-2-yl]methylamino]-4,4-dimethyl-pentoxy]-6-(2,6-dimethylphenyl)pyrimidin-2-yl]sulfamoyl]benzoic acid (hydrochloride salt) (158 mg, 0.2111 mmol) in anhydrous DMF (8 mL) was added 2-chloro-4,6-dimethoxy-1,3,5-triazine (50 mg, 0.2848 mmol) (CDMT), followed by addition of 4-methylmorpholine (150 μL, 1.364 mmol) at 0-4° C. (ice-water bath) under nitrogen. The clear reaction was allowed to stir at that temperature for 15 min, then allowed to stir at room temperature for several h. The product was purified by preparative reverse-phase HPLC (1-99% acetonitrile in water over 15 min, HCl as modifier). The combined fractions were extracted with ethyl acetate (3×20 mL), washed with brine (20 mL), dried over sodium sulfate, filtered and concentrated and dried under reduced pressure to furnish tert-butyl (2R)-2-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]morpholine-4-carboxylate (102 mg, 69%) as a white solid. ESI-MS m/z calc. 693.31964, found 694.3 (M+1)⁺; Retention time: 1.93 minutes; LC method A. ¹H NMR (400 MHz, DMSO-d₆) δ 13.07 (s, 1H), 8.49 (s, 1H), 7.95 (s, 1H), 7.68 (s, 2H), 7.25 (d, J 7.9 Hz, 1H), 7.13 (s, 2H), 6.41 (s, 1H), 5.07 (dd, J 10.5, 4.3 Hz, 1H), 4.26 (t, J 11.0 Hz, 1H), 3.96 (d, J 12.9 Hz, 1H), 3.88 (d, J 11.3 Hz, 1H), 3.85-3.65 (m, 3H), 3.57 (dd, J 14.2, 5.4 Hz, 1H), 3.41 (td, J 11.6, 2.8 Hz, 1H), 3.31-3.26 (m, 1H), 2.81 (d, J 72.4 Hz, 2H), 2.30-1.64 (m, 7H), 1.40 (s, 9H), 1.31 (d, J 15.0 Hz, 1H), 0.49 (s, 9H).

### Example 118: Preparation of Compound 142

### Step 1: (11R)-6-(2,6-Dimethylphenyl)-11-(2,2-dimethylpropyl)-12-{[(2S)-morpholin-2-yl]methyl}-9-oxa-2λ⁶-thia-3,5,12,19-tetraazatricyclo[12.3.1.14,8]nonadeca-1(17),4,6,8(19),14(18),15-hexaene-2,2,13-trione

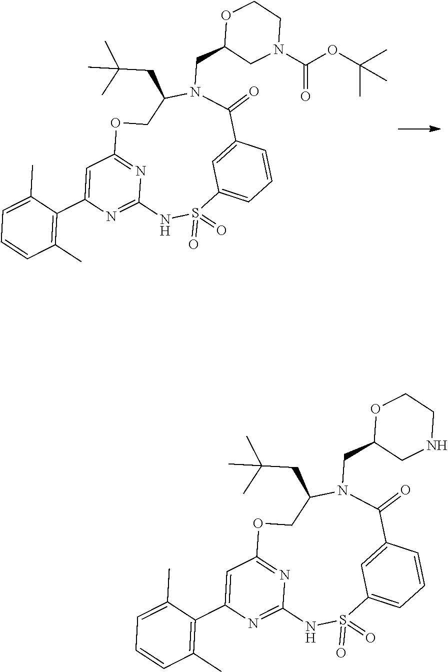 [SMILES: Cc1cccc(C)c1-c1cc2nc(n1)NS(=O)(=O)c1cccc(c1)C(=O)N(C[C@@H]1CN(C(=O)OC(C)(C)C)CCO1)[C@H](CC(C)(C)C)CO2 ; Cc1cccc(C)c1-c1cc2nc(n1)NS(=O)(=O)c1cccc(c1)C(=O)N(C[C@@H]1CNCCO1)[C@H](CC(C)(C)C)CO2]

To a stirred solution of tert-butyl (2R)-2-{[(11R)-6-(2,6-dimethylphenyl)-11-(2,2-dimethylpropyl)-2,2,13-trioxo-9-oxa-2λ⁶-thia-3,5,12,19-tetraazatricyclo[12.3.1.14,8]nonadeca-1(17),4,6,8(19),14(18),15-hexaen-12-yl]methyl}morpholine-4-carboxylate (90 mg, 0.1284 mmol) in anhydrous dichloromethane (2 mL) was added hydrogen chloride (in dioxane) (600 μL of 4 M, 2.400 mmol) and the reaction was allowed to stir at room temperature under nitrogen for 2 h. The volatiles were removed under reduced pressure to furnish (11R)-6-(2,6-dimethylphenyl)-11-(2,2-dimethylpropyl)-12-{[(2S)-morpholin-2-yl]methyl}-9-oxa-2λ⁶-thia-3,5,12,19-tetraazatricyclo[12.3.1.14,8]nonadeca-1(17),4,6,8(19),14(18),15-hexaene-2,2,13-trione (hydrochloride salt) (81 mg, 100%) as white solid. It was used in the subsequent reaction without further purification. ESI-MS m/z calc. 593.2672, found 594.4 (M+1)⁺; Retention time: 1.21 minutes; LC method A.

### Step 2: (11R)-6-(2,6-Dimethylphenyl)-11-(2,2-dimethylpropyl)-12-{[(2S)-4-(2,2-dimethylpropyl)morpholin-2-yl]methyl}-9-oxa-2λ⁶-thia-3,5,12,19-tetraazatricyclo[12.3.1.14,8]nonadeca-1(17),4,6,8(19),14(18),15-hexaene-2,2,13-trione (Compound 142)

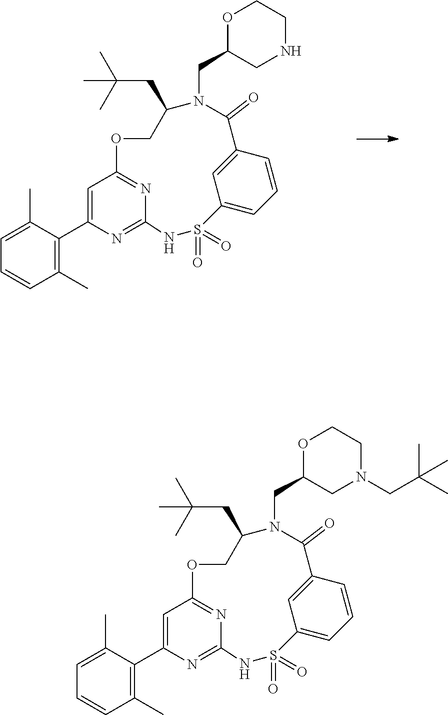 [SMILES: Cc1cccc(C)c1-c1cc2nc(n1)NS(=O)(=O)c1cccc(c1)C(=O)N(C[C@@H]1CNCCO1)[C@H](CC(C)(C)C)CO2 ; Cc1cccc(C)c1-c1cc2nc(n1)NS(=O)(=O)c1cccc(c1)C(=O)N(C[C@@H]1CN(CC(C)(C)C)CCO1)[C@H](CC(C)(C)C)CO2]

To a stirred solution of (11R)-6-(2,6-dimethylphenyl)-11-(2,2-dimethylpropyl)-12-{[(2S)-morpholin-2-yl]methyl}-9-oxa-2λ⁶-thia-3,5,12,19-tetraazatricyclo[12.3.1.14,8]nonadeca-1(17),4,6,8(19),14(18),15-hexaene-2,2,13-trione (hydrochloride salt) (14 mg, 0.02222 mmol) and DIEA (20 μL, 0.1148 mmol) in anhydrous dichloromethane (0.3 mL) were added a solution of 2,2-dimethylpropanal (4 mg, 0.04644 mmol) in anhydrous dichloromethane (0.1 mL), and glacial acetic acid (5 μL, 0.08792 mmol), in that order, at room temperature. After stirring the clear solution for 15 min, sodium triacetoxyborohydride (25 mg, 0.1180 mmol) was added. The heterogeneous mixture was stirred for 2 h, then methanol (0.3 mL) and water (0.2 mL) were added. The volatiles were removed under reduced pressure and the residue was purified by preparative reverse-phase HPLC (1-99% acetonitrile in water over 30 min, 5 mM HCl as modifier) to furnish (11R)-6-(2,6-dimethylphenyl)-11-(2,2-dimethylpropyl)-12-{[(2S)-4-(2,2-dimethylpropyl)morpholin-2-yl]methyl}-9-oxa-2λ⁶-thia-3,5,12,19-tetraazatricyclo[12.3.1.14,8]nonadeca-1(17),4,6,8(19),14(18),15-hexaene-2,2,13-trione (hydrochloride salt) (10 mg, 64%) as a white solid. ESI-MS m/z calc. 663.34546, found 664.5 (M+1)⁺; Retention time: 1.35 minutes; LC method A. ¹H NMR (400 MHz, DMSO-d₆) δ 13.03 (s, 1H), 9.66 (s, 1H), 8.53 (s, 1H), 7.96 (d, J 7.6 Hz, 1H), 7.88-7.53 (m, 2H), 7.26 (t, J 7.6 Hz, 1H), 7.13 (d, J 7.7 Hz, 2H), 6.42 (s, 1H), 5.11 (dd, J 10.8, 4.4 Hz, 1H), 4.54-4.38 (m, 1H), 4.24 (t, J=11.3 Hz, 1H), 4.16-3.95 (m, 2H), 3.84 (q, J=8.1, 7.0 Hz, 1H), 3.72-3.55 (m, 2H), 3.52-3.43 (m, 2H), 3.22-3.03 (m, 4H), 1.97 (s, 6H), 1.90-1.81 (m, 1H), 1.33 (d, J 15.0 Hz, 1H), 1.11 (s, 9H), 0.50 (s, 9H).

### Example 119: Preparation of Compound 143

### Step 1: 3-[[4-[(2R)-2-[(4-Bromophenyl)methylamino]-4,4-dimethyl-pentoxy]-6-(2,6-dimethylphenyl)pyrimidin-2-yl]sulfamoyl]benzoic acid

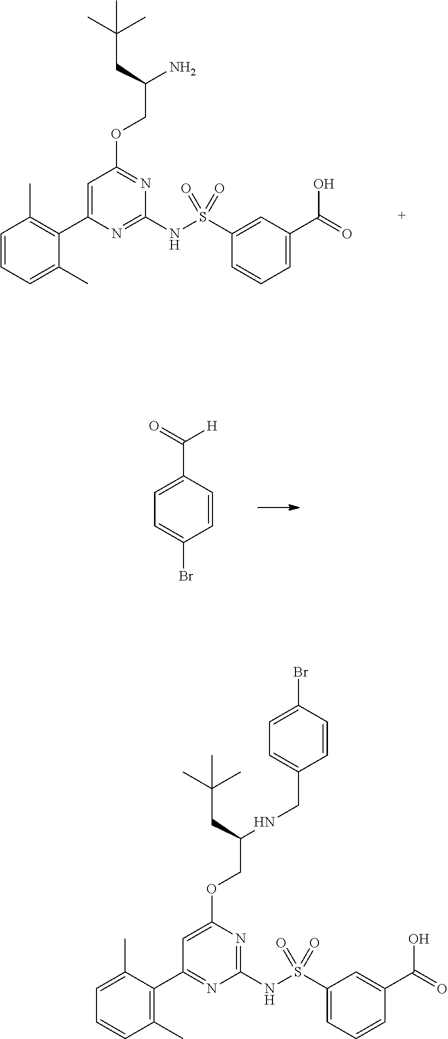 [SMILES: Cc1cccc(C)c1-c1cc(OC[C@H](N)CC(C)(C)C)nc(NS(=O)(=O)c2cccc(C(=O)O)c2)n1 ; O=Cc1ccc(Br)cc1 ; Cc1cccc(C)c1-c1cc(OC[C@@H](CC(C)(C)C)NCc2ccc(Br)cc2)nc(NS(=O)(=O)c2cccc(C(=O)O)c2)n1]

In a reaction vial, 3-[[4-[(2R)-2-amino-4,4-dimethyl-pentoxy]-6-(2,6-dimethylphenyl)pyrimidin-2-yl]sulfamoyl]benzoic acid (hydrochloride salt) (1 g, 1.821 mmol) was mixed with 4-bromobenzaldehyde (505.5 mg, 2.732 mmol) in methylene chloride (4.8 mL). The reaction mixture was stirred at rt for 15 min then sodium triacetoxyborohydride (1.0 g, 4.718 mmol) was added. The reaction was stirred at rt for 3 h. then partitioned between ethyl acetate and 1N HCl. The reaction mixture was extracted with ethyl acetate (3×) and the organic layer was washed with saturated NaCl solution. The organic layer was isolated, dried over anhydrous sodium sulfate, filtered, and evaporated to dryness. The crude material was purified by recrystallization from ethyl acetate/hexanes. The product was isolated as a white solid. 3-[[4-[(2R)-2-[(4-bromophenyl)methylamino]-4,4-dimethyl-pentoxy]-6-(2,6-dimethylphenyl)pyrimidin-2-yl]sulfamoyl]benzoic acid (hydrochloride salt) (1.1243 g, 82%) ESI-MS m/z calc. 680.1668, found 683.2 (M+3)+; Retention time: 1.48 minutes (LC method A).

### Step 2: (11R)-12-[(4-Bromophenyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

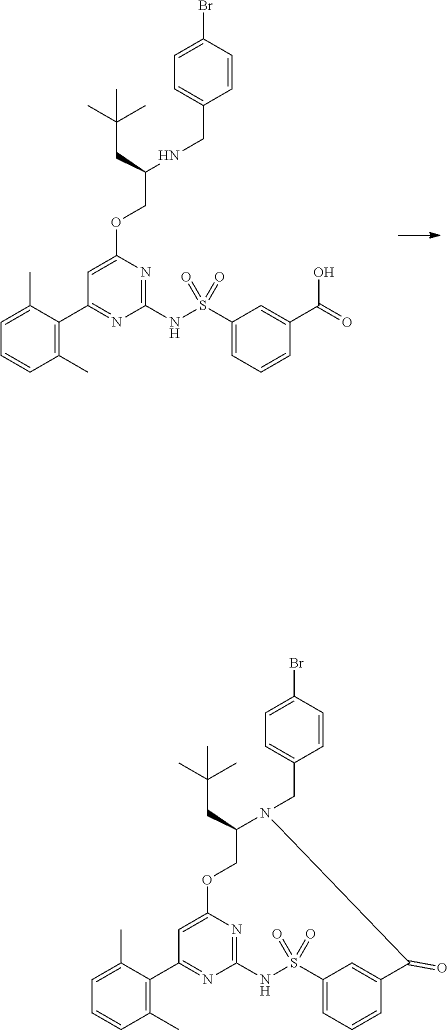 [SMILES: Cc1cccc(C)c1-c1cc(OC[C@@H](CC(C)(C)C)NCc2ccc(Br)cc2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccc(Br)cc1)[C@H](CC(C)(C)C)CO2]

In a reaction vial, 3-[[4-[(2R)-2-[(4-bromophenyl)methylamino]-4,4-dimethyl-pentoxy]-6-(2,6-dimethylphenyl)pyrimidin-2-yl]sulfamoyl]benzoic acid (hydrochloride salt) (1.124 g, 1.487 mmol) was dissolved in DMF (50.3 mL) along with 4-methylmorpholine (327 μL, 2.974 mmol) and cooled to 0° C. To the reaction, 2-chloro-4,6-dimethoxy-1,3,5-triazine (261.1 mg, 1.487 mmol) was added and the reaction was allowed to stir at 0° C. for 1.5 h. then additional 4-methylmorpholine (164 μL, 1.492 mmol) was added. The reaction was allowed to warm to rt and stirred at that temperature overnight. The reaction was concentrated to a third of the volume then partitioned between ethyl acetate and 0.5N HCl solution. The organics were separated, dried over sodium sulfate, and evaporated to dryness. The crude material was purified by column chromatography on silica using 20-80% ethyl acetate/hexanes gradient. The product was isolated as a white solid. (11R)-12-[(4-bromophenyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (560.6 mg, 56%). ¹H NMR (400 MHz, DMSO-d₆) δ 13.07 (s, 1H), 12.33 (s, 1H), 8.52 (s, 1H), 7.98 (d, J 6.4 Hz, 1H), 7.72 (s, 2H), 7.63-7.55 (m, 2H), 7.43-7.37 (m, 2H), 7.26 (t, J 7.6 Hz, 1H), 7.21-7.09 (m, 2H), 6.43 (s, 1H), 5.09 (dd, J 10.4, 4.3 Hz, 1H), 4.77 (d, J 15.6 Hz, 1H), 4.52 (d, J 15.6 Hz, 1H), 4.25 (t, J 11.1 Hz, 1H), 4.06-3.95 (m, 1H), 2.01-1.92 (m, 4H), 1.80 (dd, J 15.3, 8.6 Hz, 1H), 1.37 (d, J 15.1 Hz, 1H), 0.50 (s, 10H). ESI-MS m/z calc. 662.15625, found 665.0 (M+3)⁺; Retention time: 2.1 minutes (LC method A).

### Step 3: (11R)-12-{[4-(3,5-dimethyl-1H-pyrazol-4-yl)phenyl]methyl}-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4,6,8(19),14(18),15-hexaene-2,2,13-trione (Compound 143)

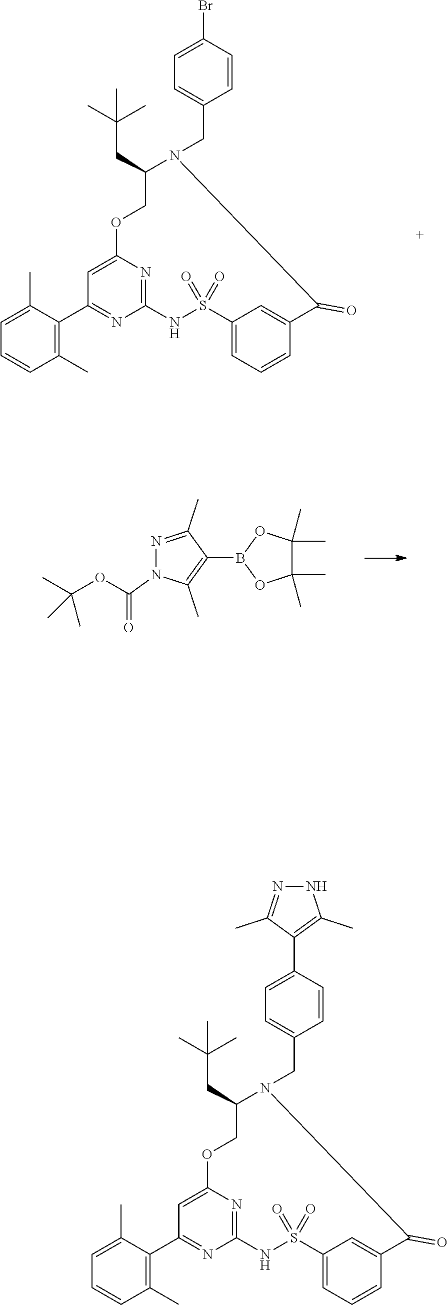 [SMILES: Cc1cccc(C)c1-c1cc2nc(n1)NS(=O)(=O)c1cccc(c1)C(=O)N(Cc1ccc(Br)cc1)[C@H](CC(C)(C)C)CO2 ; Cc1nn(C(=O)OC(C)(C)C)c(C)c1B1OC(C)(C)C(C)(C)O1 ; Cc1cccc(C)c1-c1cc2nc(n1)NS(=O)(=O)c1cccc(c1)C(=O)N(Cc1ccc(-c3c(C)n[nH]c3C)cc1)[C@H](CC(C)(C)C)CO2]

In a microwave reaction vial, (11R)-12-[(4-bromophenyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (30 mg, 0.04475 mmol) was mixed with the specified boronic acid/ester (tert-butyl 3,5-dimethyl-4-(4,4,5,5-tetramethyl-1,3,2-dioxaborolan-2-yl)pyrazole-1-carboxylate (16.2 mg, 0.05028 mmol)) and potassium carbonate (70.5 μL of 2 M, 0.1410 mmol) in DMSO (600 μL). The reaction was flushed with nitrogen then Pd(dppf)Cl₂ (3 mg, 0.003674 mmol) was added. The reaction was purged again with nitrogen and heated at 120° C. for 45 min in the microwave. The reaction mixture was diluted with ethyl acetate and washed with 1N HCl followed by saturated NaCl solution. The organic layer was isolated, dried over anhydrous sodium sulfate, filtered, and evaporated to dryness. The crude material was purified by preparative HPLC to give (11R)-12-{[4-(3,5-dimethyl-1H-pyrazol-4-yl)phenyl]methyl}-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4,6,8(19),14(18),15-hexaene-2,2,13-trione (13.3 mg, 44%) ESI-MS m/z calc. 678.2988, found 679.3 (M+1)⁺; Retention time: 1.64 minutes; LC method A.

### Example 120: Preparation of Compound 144 and Compound 145

### Step 1: 3-[[4-(2,6-Dimethylphenyl)-6-[(2R)-2-[(4-iodophenyl)methylamino]-4,4-dimethyl-pentoxylpyrimidin-2-yl]sulfamoyl]benzoic acid

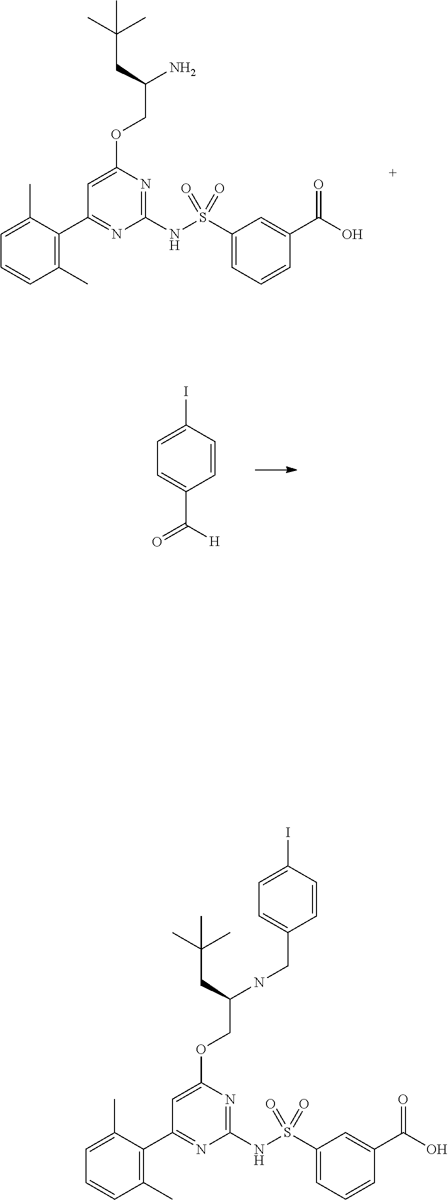 [SMILES: Cc1cccc(C)c1-c1cc(OC[C@H](N)CC(C)(C)C)nc(NS(=O)(=O)c2cccc(C(=O)O)c2)n1 ; O=Cc1ccc(I)cc1 ; Cc1cccc(C)c1-c1cc(OC[C@@H](CC(C)(C)C)NCc2ccc(I)cc2)nc(NS(=O)(=O)c2cccc(C(=O)O)c2)n1]

In a 20 mL vial, to a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (300 mg, 0.5464 mmol) and 4-iodobenzaldehyde (128 mg, 0.5517 mmol) in anhydrous dichloromethane (5 mL) were added glacial acetic acid (40 μL, 0.7034 mmol), sodium triacetoxyborohydride (1.2 g, 5.662 mmol), and DIEA (0.4 mL, 2.296 mmol), in that order. The vial was purged with nitrogen briefly and capped and allowed to stir at ambient temperature for 4 h. Then methanol (1.0 mL) and water (0.4 mL) were added in that order, and the volatiles were removed under reduced pressure. The residue was taken up in DMSO (3 mL), micro-filtered, and purified by reverse-phase HPLC (1-99% acetonitrile in water over 15 min, HCl as modifier) to give 3-[[4-(2,6-dimethylphenyl)-6-[(2R)-2-[(4-iodophenyl)methylamino]-4,4-dimethyl-pentoxy]pyrimidin-2-yl]sulfamoyl]benzoic acid (hydrochloride salt) (219 mg, 52%) as white solid. ¹H NMR (400 MHz, DMSO-d₆) δ 8.45 (t, J=1.8 Hz, 1H), 8.20-8.07 (m, 2H), 7.78 (d, J=8.1 Hz, 2H), 7.68 (t, J 7.8 Hz, 1H), 7.34 (d, J 8.0 Hz, 2H), 7.26 (t, J 7.6 Hz, 1H), 7.13 (d, J 7.6 Hz, 2H), 6.36 (s, 1H), 4.37 (d, J 12.1 Hz, 1H), 4.25 (dd, J 12.5, 5.7 Hz, 1H), 4.20-4.08 (m, 2H), 3.46-3.40 (m, 3H), 2.02 (s, 6H), 1.74-1.55 (m, 2H), 0.90 (s, 9H). ESI-MS m/z calc. 728.15295, found 729.2 (M+1)⁺; Retention time: 1.39 minutes (LC method A).

### Step 2: (11R)-6-(2,6-Dimethylphenyl)-11-(2,2-dimethylpropyl)-12-[(4-iodophenyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

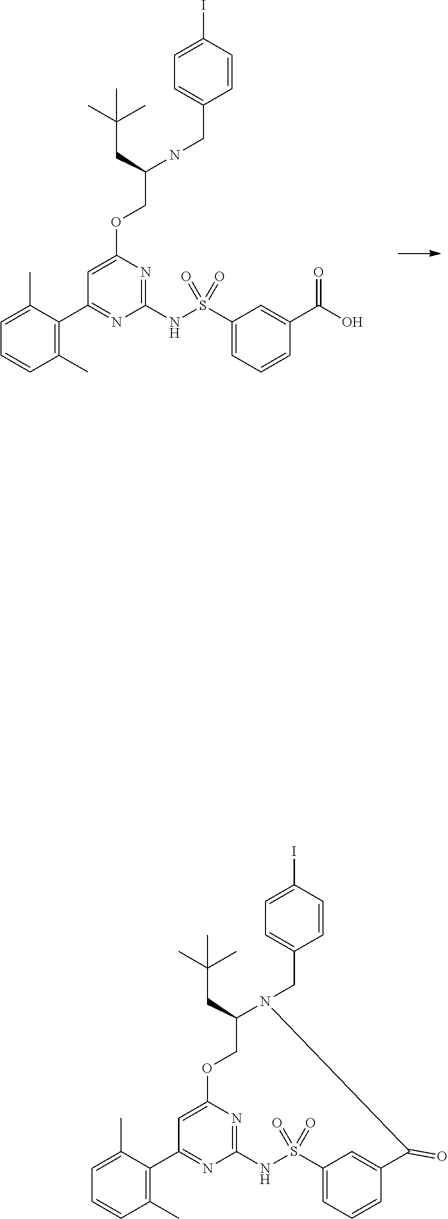 [SMILES: Cc1cccc(C)c1-c1cc(OC[C@@H](CC(C)(C)C)NCc2ccc(I)cc2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccc(I)cc1)[C@H](CC(C)(C)C)CO2]

In a 25 mL flask, to a stirred solution of 3-[[4-(2,6-dimethylphenyl)-6-[(2R)-2-[(4-iodophenyl)methylamino]-4,4-dimethyl-pentoxy]pyrimidin-2-yl]sulfamoyl]benzoic acid (hydrochloride salt) (100 mg, 0.1307 mmol) in anhydrous DMF (5 mL) was added [dimethylamino(triazolo[4,5-b]pyridin-3-yloxy)methylene]-dimethyl-ammonium (Phosphorus Hexafluoride Ion) (82 mg, 0.2157 mmol) (HATU), followed by addition of DIEA (120 μL, 0.6889 mmol) at ambient temperature. The vial was briefly purged with nitrogen and the capped vial was allowed to stir at ambient temperature for 15 h (overnight). The orange solution was micro-filtered, and purified by preparative reverse-phase HPLC (1-99% acetonitrile in water over 15 min, 5 mM HCl as modifier) to furnish (11R)-6-(2,6-dimethylphenyl)-11-(2,2-dimethylpropyl)-12-[(4-iodophenyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (30 mg, 32%) as white solid. It would be used in the subsequent reaction. ESI-MS m/z calc. 710.1424, found 711.3 (M+1)⁺; Retention time: 2.08 minutes (LC method A).

### Step 3: (11R)-6-(2,6-dimethylphenyl)-11-(2,2-dimethylpropyl)-2,2-dioxo-12-[(4-pyrimidin-5-ylphenyl)methyl]-9-oxa-2λ⁶-thia-3,5,12,19-tetrazatricyclo[12.3.1.14,8]nonadeca-1(18),4(19),5,7,14,16-hexaen-13-one, (Compound 144), and (11R)-6-(2,6-dimethylphenyl)-11-(2,2-dimethylpropyl)-12-[[4-(2-hydroxypyrimidin-5-yl)phenyl]methyl]-2,2-dioxo-9-oxa-2×⁶-thia-3,5,12,19-tetrazatricyclo[12.3.1.14,8]nonadeca-1(18),4(19),5,7,14,16-hexaen-13-one, (Compound 145)

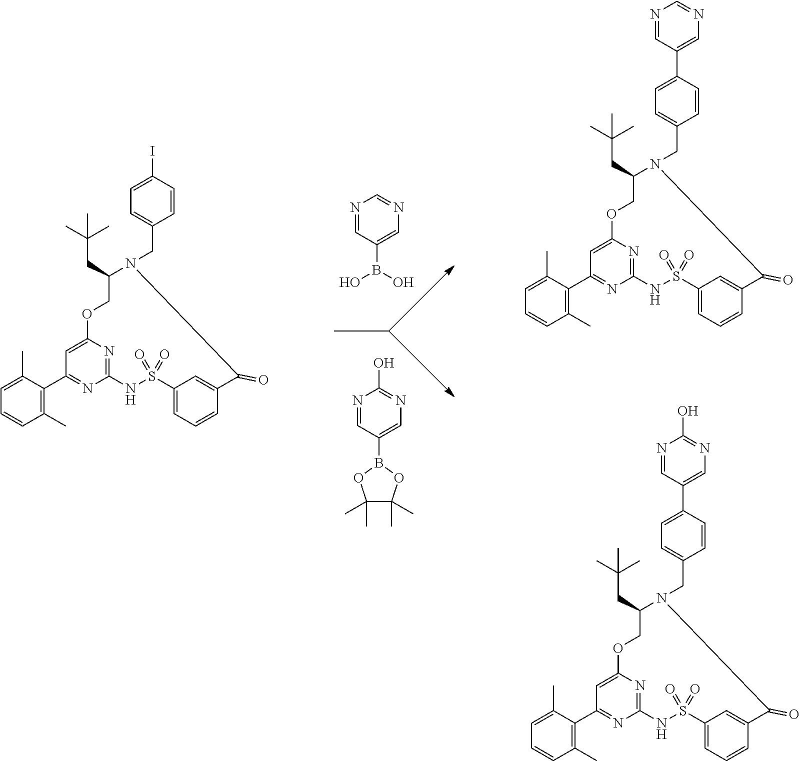

Two reactions were run in parallel: In a 4 mL vial, to a stirred solution of (11R)-6-(2,6-dimethylphenyl)-11-(2,2-dimethylpropyl)-12-[(4-iodophenyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (15 mg, 0.02111 mmol) in anhydrous dioxane (0.5 mL) were added pyrimidin-5-ylboronic acid (7 mg, 0.05649 mmol), and potassium carbonate (12 mg, 0.08683 mmol). Nitrogen gas was purged through the vial for 2 min. Then tetrakis(triphenylphosphane)palladium(0) (3 mg, 0.002596 mmol) was added and the vial was flushed with nitrogen for another 2 min, sealed with a screw cap under nitrogen and heated at 70° C. (external temperature) for 15 h. The reaction was allowed to cool to ambient temperature and the dark reaction mixture was diluted with DMSO (1 mL), filtered through a syringe filter disc and purified by preparative reverse phase HPLC-MS method using a Luna C₁₈(2) column (75×30 mm, 5 μm particle size) sold by Phenomenex (pn: 00C-4252-U0-AX), and a dual gradient run from 1-99% acetonitrile in water over 15.0 minutes (5 mM HCl as modifier) to give (11R)-6-(2,6-dimethylphenyl)-11-(2,2-dimethylpropyl)-2,2-dioxo-12-[(4-pyrimidin-5-ylphenyl)methyl]-9-oxa-2λ⁶-thia-3,5,12,19-tetrazatricyclo[12.3.1.14,8]nonadeca-1(18),4(19),5,7,14,16-hexaen-13-one (11 mg, 78%) as white solid. ¹H NMR (400 MHz, DMSO-d₆) δ 13.09 (s, 1H), 9.23-9.13 (m, 3H), 8.56 (s, 1H), 7.98 (s, 1H), 7.90-7.82 (m, 2H), 7.73 (s, 2H), 7.59 (d, J 8.1 Hz, 2H), 7.26 (t, J 7.6 Hz, 1H), 7.12 (d, J 7.6 Hz, 2H), 6.43 (s, 1H), 5.14 (dd, J 10.4, 4.3 Hz, 1H), 4.90 (d, J 15.6 Hz, 1H), 4.59 (d, J=15.7 Hz, 1H), 4.29 (t, J=11.0 Hz, 1H), 4.14-3.97 (m, 1H), 2.23-1.78 (m, 7H), 1.41 (d, J 15.1 Hz, 1H), 0.54 (s, 9H). ESI-MS m/z calc. 662.2675, found 663.3 (M+1)⁺; Retention time: 1.75 minutes (LC method A).

Using the above protocol with 5-(4,4,5,5-tetramethyl-1,3,2-dioxaborolan-2-yl)pyrimidin-2-ol (11 mg, 0.04954 mmol), the desired (11R)-6-(2,6-dimethylphenyl)-11-(2,2-dimethylpropyl)-12-[[4-(2-hydroxypyrimidin-5-yl)phenyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (11 mg, 76%) was obtained as white solid. ¹H NMR (400 MHz, DMSO-d₆) δ 13.04 (s, 1H), 8.65 (s, 2H), 8.52 (s, 1H), 7.98 (d, J 6.5 Hz, 1H), 7.82-7.69 (m, 2H), 7.69-7.61 (m, 2H), 7.49 (d, J 8.1 Hz, 2H), 7.26 (t, J 7.6 Hz, 1H), 7.13 (d, J 7.7 Hz, 2H), 6.43 (s, 1H), 5.09 (dd, J 10.4, 4.2 Hz, 1H), 4.88 (d, J 15.5 Hz, 1H), 4.52 (d, J 15.6 Hz, 1H), 4.25 (t, J 11.0 Hz, 1H), 4.08-3.98 (m, 1H), 1.96 (s, 6H), 1.86 (dd, J 15.3, 8.7 Hz, 1H), 1.40 (d, J 15.0 Hz, 1H), 0.53 (s, 9H). ESI-MS m/z calc. 678.26245, found 679.4 (M+1)⁺; Retention time: 1.53 minutes (LC method A).

### Example 121: Preparation of Compound 146

### Step 1: 1-[4-[[(11R)-6-(2,6-Dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]phenyl]imidazolidine-2,4-dione (Compound 146)

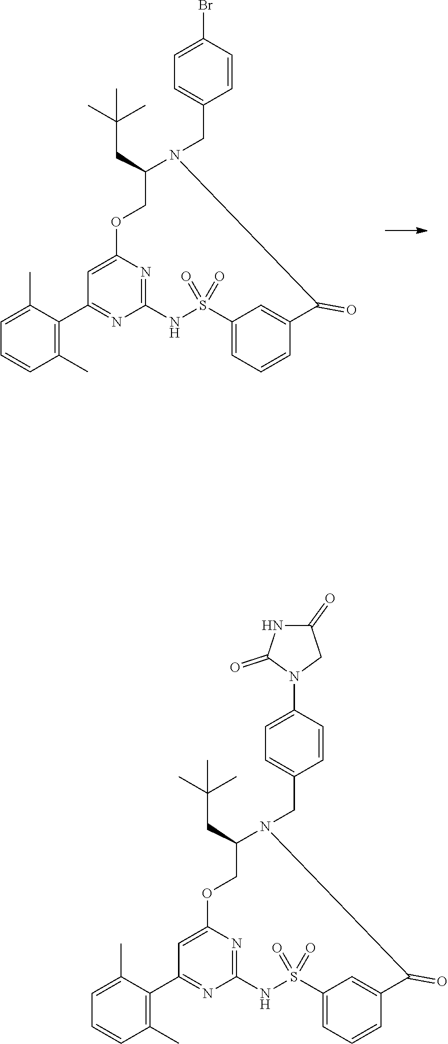 [SMILES: Cc1cccc(C)c1-c1cc2nc(n1)NS(=O)(=O)c1cccc(c1)C(=O)N(Cc1ccc(Br)cc1)[C@H](CC(C)(C)C)CO2 ; Cc1cccc(C)c1-c1cc2nc(n1)NS(=O)(=O)c1cccc(c1)C(=O)N(Cc1ccc(N3CC(=O)NC3=O)cc1)[C@H](CC(C)(C)C)CO2]

In a reaction vial, (11R)-12-[(4-bromophenyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (67 mg, 0.09995 mmol) was dissolved in DMSO (0.5 mL) along with imidazolidine-2,4-dione (50 mg, 0.4996 mmol), iodocopper (11.4 mg, 0.05986 mmol), 2-(dimethylamino)acetic acid (hydrochloride salt) (11.2 mg, 0.08024 mmol), and potassium carbonate (41.4 mg, 0.2996 mmol). The reaction mixture was heated at 120° C. for 12 h. then cooled to rt The reaction mixture was partitioned between ethyl acetate and 1N HCl. The reaction mixture was extracted with ethyl acetate and the combined organic layers were washed with saturated NaCl solution. The organic layer was isolated, dried over anhydrous sodium sulfate, filtered, and evaporated to dryness. The crude material was purified by preparative HPLC to provide the product as a white solid, 1-[4-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]phenyl]imidazolidine-2,4-dione (22.9 mg, 34%) ESI-MS m/z calc. 682.2573, found 683.3 (M+1)⁺; Retention time: 1.62 minutes (LC method A).

### Example 122: Preparation of Compound 147

### Step 1: (11R)-12-[(2-bromophenyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

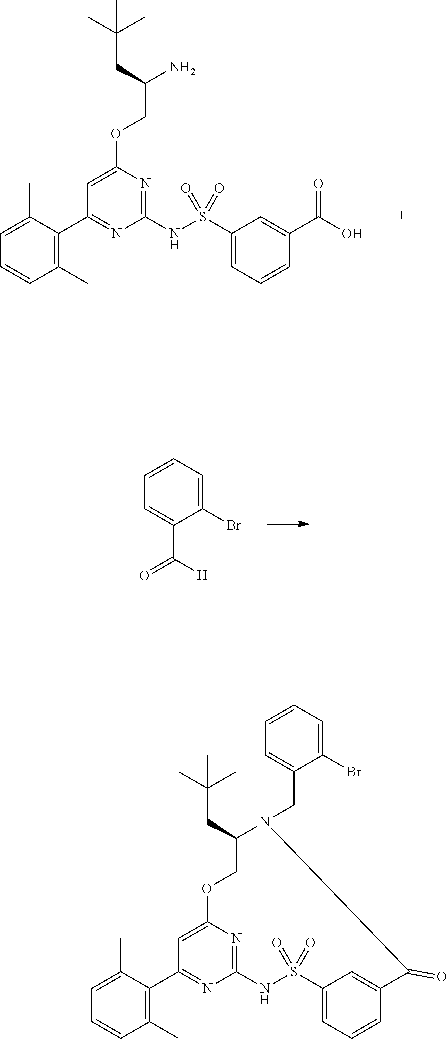 [SMILES: Cc1cccc(C)c1-c1cc(OC[C@H](N)CC(C)(C)C)nc(NS(=O)(=O)c2cccc(C(=O)O)c2)n1 ; O=Cc1ccccc1Br ; Cc1cccc(C)c1-c1cc2nc(n1)NS(=O)(=O)c1cccc(c1)C(=O)N(Cc1ccccc1Br)[C@H](CC(C)(C)C)CO2]

In a reaction vial, 3-[[4-[(2R)-2-amino-4,4-dimethyl-pentoxy]-6-(2,6-dimethylphenyl)pyrimidin-2-yl]sulfamoyl]benzoic acid (hydrochloride salt) (1 g, 1.821 mmol) was mixed with 2-bromobenzaldehyde (505.5 mg, 2.732 mmol) in methylene chloride (4.8 mL). The reaction mixture was stirred at rt for 15 min then sodium triacetoxyborohydride (1.004 g, 4.737 mmol) was added. The reaction was stirred at rt for 3 h. then additional amounts of 2-bromobenzaldehyde (336.9 mg, 1.821 mmol) and sodium triacetoxyborohydride (385.9 mg, 1.821 mmol) were added. The reaction was allowed to stir at rt for another 3 h. The reaction mixture was then partitioned between ethyl acetate and 1N HCl. The reaction mixture was extracted with ethyl acetate (3×) and the organic layer was washed with saturated NaCl solution. The organic layer was isolated, dried over anhydrous sodium sulfate, filtered, and evaporated to dryness. The crude material was purified by recrystallization from ethyl acetate/hexanes. The intermediate was isolated as a white solid and used for the next reaction without further purification. In a reaction vial, the intermediate was dissolved in dimethylformamide (36 mL) along with 4-methylmorpholine (205.1 mg, 2.028 mmol) and cooled to 0° C. To the reaction, 2-chloro-4,6-dimethoxy-1,3,5-triazine (178 mg, 1.014 mmol) was added and the reaction was allowed to stir at 0° C. for 1 h. then additional 4-methylmorpholine (102.6 mg, 1.014 mmol) was added. The reaction was allowed to warm to rt and stirred at that temperature overnight. The reaction was concentrated to a third of the volume then partitioned between ethyl acetate and 1N HCl solution. The organics were separated, dried over sodium sulfate, and evaporated to dryness. The crude material was purified by column chromatography on silica using 20-80% ethyl acetate/hexanes gradient to give (11R)-12-[(2-bromophenyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (259.6 mg, 21%). ¹H NMR (400 MHz, DMSO-d₆) δ 13.10 (s, 1H), 8.66 (s, 1H), 8.00 (d, J 7.1 Hz, 1H), 7.74 (d, J 7.6 Hz, 2H), 7.69 (dd, J 8.0, 1.2 Hz, 1H), 7.47 (td, J 7.5, 1.2 Hz, 1H), 7.41 (dd, J 7.8, 1.8 Hz, 1H), 7.26 (td, J 7.6, 1.7 Hz, 2H), 7.12 (d, J 7.6 Hz, 2H), 6.42 (s, 1H), 5.16 (d, J 6.2 Hz, 1H), 4.92 (d, J 16.2 Hz, 1H), 4.52 (d, J 16.2 Hz, 1H), 4.10-3.96 (m, 2H), 2.07 (s, 3H), 1.97 (d, J=17.0 Hz, 4H), 1.83-1.73 (m, 1H), 1.41 (d, J=15.1 Hz, 1H), 0.53 (s, 8H). ESI-MS m/z calc. 662.15625, found 665.1 (M+3)+; Retention time: 2.16 minutes (LC method A).

### Step 2: (11R)-6-(2,6-dimethylphenyl)-11-(2,2-dimethylpropyl)-12-{[2-(1H-pyrazol-4-yl)phenyl]methyl}-9-oxa-2λ⁶-thia-3,5,12,19-tetraazatricyclo[12.3.1.14,8]nonadeca-1(17),4,6,8(19),14(18),15-hexaene-2,2,13-trione (Compound 147)

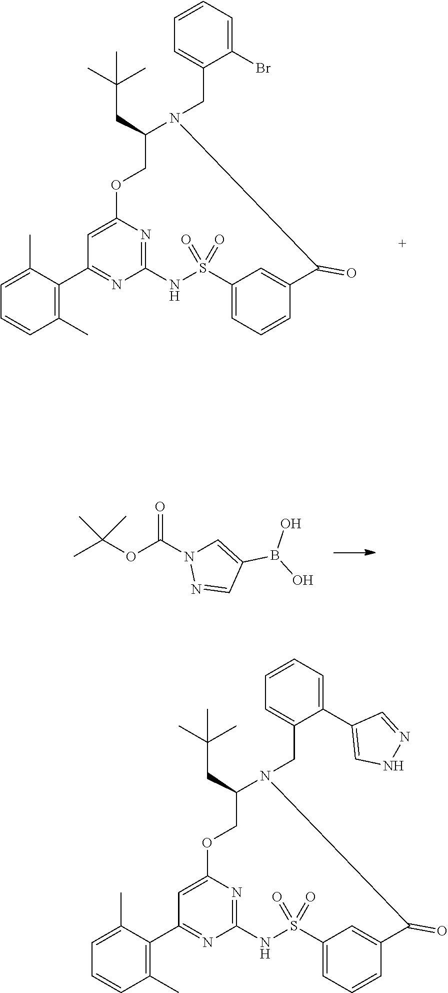 [SMILES: Cc1cccc(C)c1-c1cc2nc(n1)NS(=O)(=O)c1cccc(c1)C(=O)N(Cc1ccccc1Br)[C@H](CC(C)(C)C)CO2 ; CC(C)(C)OC(=O)n1cc(B(O)O)cn1 ; Cc1cccc(C)c1-c1cc2nc(n1)NS(=O)(=O)c1cccc(c1)C(=O)N(Cc1ccccc1-c1cn[nH]c1)[C@H](CC(C)(C)C)CO2]

In a microwave reaction vial, (11R)-12-[(2-bromophenyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (30 mg, 0.04521 mmol) was mixed with the specified boronic acid/ester ((1-tert-butoxycarbonylpyrazol-4-yl)boronic acid (approximately 10.78 mg, 0.05086 mmol)) and potassium carbonate (70.5 μL of 2 M, 0.1410 mmol) in DMSO (600 μL). The reaction was flushed with nitrogen then Pd(dppf)Cl₂ (3 mg, 0.003674 mmol) was added. The reaction was purged again with nitrogen and heated at 120° C. for 45 min in the microwave. The reaction mixture was diluted with ethyl acetate and washed with 1N HCl followed by saturated NaCl solution. The organic layer was isolated, dried over anhydrous sodium sulfate, filtered, and evaporated to dryness. The crude material was purified by preparative HPLC to give (11R)-6-(2,6-dimethylphenyl)-11-(2,2-dimethylpropyl)-12-{[2-(1H-pyrazol-4-yl)phenyl]methyl}-9-oxa-2λ⁶-thia-3,5,12,19-tetraazatricyclo[12.3.1.14,8]nonadeca-1(17),4,6,8(19),14(18),15-hexaene-2,2,13-trione (13.1 mg, 45%) ESI-MS m/z calc. 650.2675, found 651.4 (M+1)⁺; Retention time: 1.71 minutes; LC method A. ¹H NMR (400 MHz, Methanol-d4) δ 8.54 (d, J 1.8 Hz, 1H), 8.04 (dd, J 6.9, 2.1 Hz, 2H), 8.03 (s, OH), 7.75 (dt, J 7.7, 1.5 Hz, 1H), 7.69 (t, J 7.7 Hz, 1H), 7.58 (d, J 7.5 Hz, 1H), 7.47-7.33 (m, 3H), 7.26 (t, J 7.6 Hz, 1H), 7.12 (d, J 7.7 Hz, 2H), 6.21 (s, 1H), 5.37 (d, J 15.8 Hz, 1H), 5.16 (dd, J 10.7, 4.2 Hz, 1H), 4.49 (d, J 15.8 Hz, 1H), 4.04 (q, J 6.5, 5.0 Hz, 1H), 3.58 (t, J 11.2 Hz, 1H), 2.05 (s, 7H), 1.68 (dd, J 15.4, 8.9 Hz, 1H), 1.34 (dd, J 15.4, 1.5 Hz, 1H), 0.57 (s, 9H).

### Example 123: Preparation of Compound 148

### Step 1: (11R)-12-[(3-Bromophenyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

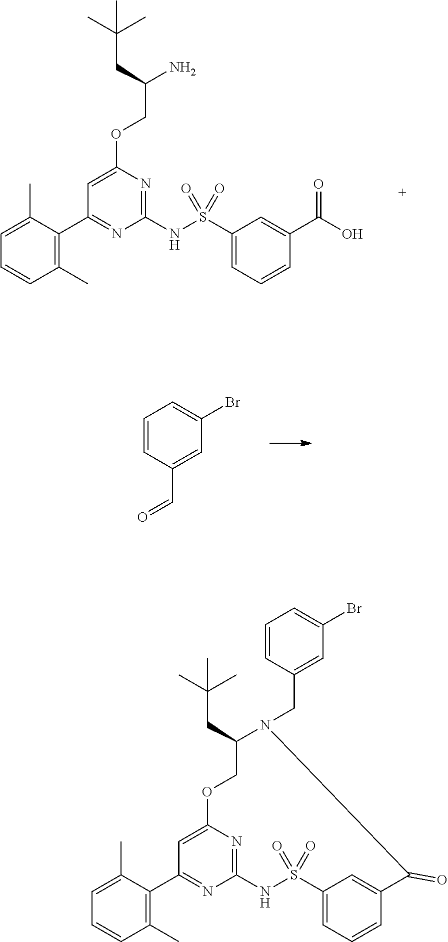 [SMILES: Cc1cccc(C)c1-c1cc(OC[C@H](N)CC(C)(C)C)nc(NS(=O)(=O)c2cccc(C(=O)O)c2)n1 ; O=Cc1cccc(Br)c1 ; Cc1cccc(C)c1-c1cc2nc(n1)NS(=O)(=O)c1cccc(c1)C(=O)N(Cc1cccc(Br)c1)[C@H](CC(C)(C)C)CO2]

In a reaction vial, 3-[[4-[(2R)-2-amino-4,4-dimethyl-pentoxy]-6-(2,6-dimethylphenyl)pyrimidin-2-yl]sulfamoyl]benzoic acid (hydrochloride salt) (1.2 g, 2.185 mmol) was mixed with 3-bromobenzaldehyde (606.5 mg, 3.278 mmol) in methylene chloride (5.76 mL). The reaction mixture was stirred at rt for 15 min then sodium triacetoxyborohydride (1.204 g, 5.681 mmol) was added. The reaction was stirred at rt for 3 h. then partitioned between ethyl acetate and 1N HCl. The reaction mixture was extracted with ethyl acetate (3×) and the organic layer was washed with saturated NaCl solution. The organic layer was isolated, dried over anhydrous sodium sulfate, filtered, and evaporated to dryness. The crude material was purified by recrystallization from ethyl acetate/hexanes. The intermediate was isolated as a white solid. In a reaction vial, the intermediate (˜1.2 g) was dissolved in dimethylformamide (58 mL) along with 4-methylmorpholine (338 mg, 3.342 mmol) and the reaction was cooled to 0° C. To the reaction, 2-chloro-4,6-dimethoxy-1,3,5-triazine (293.4 mg, 1.671 mmol) was added and the reaction was allowed to stir at 0° C. for 1 h. then additional 4-methylmorpholine (169 mg, 1.671 mmol) was added. The reaction was allowed to warm to rt and stirred at that temperature overnight. The reaction was concentrated to a third of the volume then partitioned between ethyl acetate and 1N HCl solution. The organics were separated, dried over sodium sulfate, and evaporated to dryness. The crude material was purified by column chromatography on silica using 20-80% ethyl acetate/hexanes gradient to give (11R)-12-[(3-bromophenyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (257 mg, 18%)¹H NMR (400 MHz, DMSO-d₆) δ 13.04 (s, 1H), 8.53 (s, 1H), 7.98 (t, J 4.7 Hz, 1H), 7.72 (d, J 4.7 Hz, 2H), 7.63 (d, J 1.9 Hz, 1H), 7.52-7.31 (m, 3H), 7.26 (t, J 7.6 Hz, 1H), 7.12 (d, J 7.6 Hz, 2H), 6.43 (s, 1H), 5.10 (dd, J 10.4, 4.3 Hz, 1H), 4.78 (d, J 15.7 Hz, 1H), 4.59 (d, J 15.7 Hz, 1H), 4.28 (t, J 11.0 Hz, 1H), 4.00 (ddd, J 14.9, 8.9, 4.1 Hz, 1H), 3.17 (d, J 5.1 Hz, 1H), 1.96 (s, 5H), 1.78 (dd, J=15.3, 8.5 Hz, 1H), 1.38 (d, J=15.0 Hz, 1H), 0.50 (s, 9H). ESI-MS m/z calc. 662.15625, found 665.2 (M+3)+; Retention time: 2.11 minutes (LC method A).

### Step 2: 1-[3-[[(11R)-6-(2,6-Dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]phenyl]imidazolidine-2,4-dione (Compound 148)

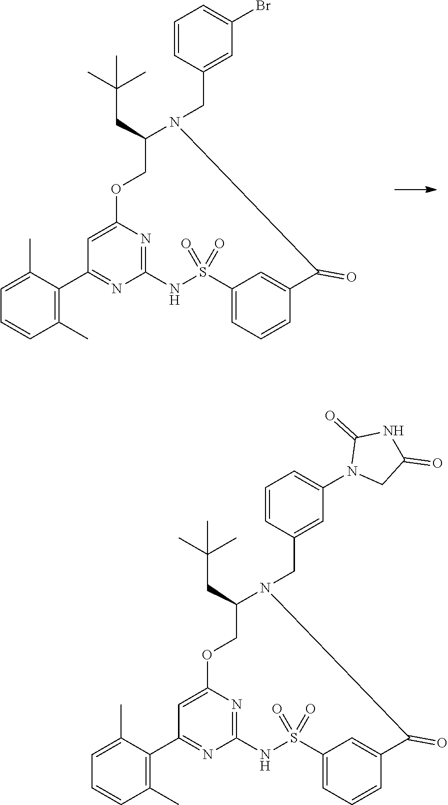 [SMILES: Cc1cccc(C)c1-c1cc2nc(n1)NS(=O)(=O)c1cccc(c1)C(=O)N(Cc1cccc(Br)c1)[C@H](CC(C)(C)C)CO2 ; Cc1cccc(C)c1-c1cc2nc(n1)NS(=O)(=O)c1cccc(c1)C(=O)N(Cc1cccc(N3CC(=O)NC3=O)c1)[C@H](CC(C)(C)C)CO2]

In a reaction vial, (11R)-12-[(3-bromophenyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (55 mg, 0.08288 mmol) was dissolved in DMSO (0.5 μL) along with imidazolidine-2,4-dione (41.5 mg, 0.4147 mmol), iodocopper (9.5 mg, 0.04988 mmol), 2-(dimethylamino)acetic acid (hydrochloride salt) (9.3 mg, 0.06663 mmol), and potassium carbonate (34.4 mg, 0.2489 mmol). The reaction mixture was heated at 120° C. for 12 h. then cooled to rt. The reaction mixture was partitioned between ethyl acetate and 1N HCl. The reaction mixture was extracted with ethyl acetate and the combined organic layers were washed with saturated NaCl solution. The organic layer was isolated, dried over anhydrous sodium sulfate, filtered, and evaporated to dryness. The crude material was purified by preparative HPLC to provide the product as a white solid, 1-[3-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]phenyl]imidazolidine-2,4-dione (12.3 mg, 22%) ESI-MS m/z calc. 682.2573, found 683.3 (M+1)⁺; Retention time: 1.62 minutes (LC method A).

### Example 124: Preparation of Compound 149 and Compound 150

### Step 1: 1,4-Dioxaspiro[4.5]decane-8-carbaldehyde

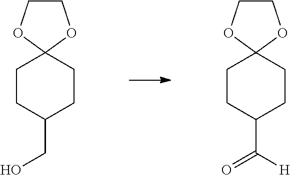 [SMILES: OCC1CCC2(CC1)OCCO2 ; O=CC1CCC2(CC1)OCCO2]

To a solution of 1,4-dioxaspiro[4.5]decan-8-ylmethanol (16.64 g, 96.619 mmol) in DCM (315 mL) was added sodium bicarbonate (34.8 g, 414.25 mmol). The reaction was cooled to 0° C. with an ice-cold bath and Dess-Martin periodinane (44.8 g, 105.63 mmol) was slowly added portionwise over 5 minutes. The reaction mixture was stirred at room temperature for 3 hours. A 5% aqueous solution of sodium bicarbonate (500 mL) was added followed by a 10% w/w aqueous solution of Na₂S₂O₃ (500 mL). The mixture was vigorously stirred at room temperature for 16 h. The phases were separated, and the aqueous layer was washed with DCM (2×100 mL). The combined organic layers were dried with sodium sulfate, filtered and concentrated under reduced pressure to afford crude 1,4-dioxaspiro[4.5]decane-8-carbaldehyde (24.77 g, 97%) as a pale-yellow oil. ESI-MS m/z calc. 170.0943, found 171.2 (M+1)⁺; Retention time: 1.31 minutes. LC method X.

### Step 2: 3-[[4-(2,6-Dimethylphenyl)-6-[(2R)-2-(1,4-dioxaspiro[4.5]decan-8-ylmethylamino)-4,4-dimethyl-pentoxy]pyrimidin-2-yl]sulfamoyl]benzoic acid

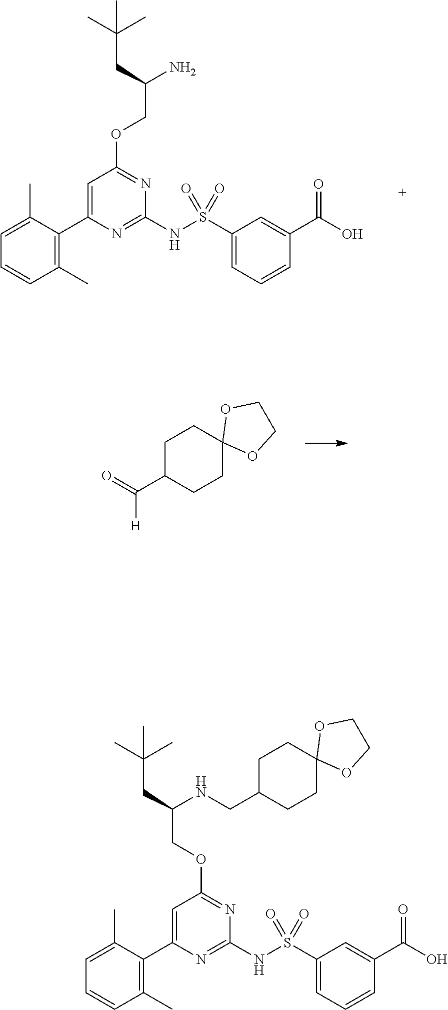 [SMILES: Cc1cccc(C)c1-c1cc(OC[C@H](N)CC(C)(C)C)nc(NS(=O)(=O)c2cccc(C(=O)O)c2)n1 ; O=CC1CCC2(CC1)OCCO2 ; Cc1cccc(C)c1-c1cc(OC[C@@H](CC(C)(C)C)NCC2CCC3(CC2)OCCO3)nc(NS(=O)(=O)c2cccc(C(=O)O)c2)n1]

To a solution of 3-[[4-[(2R)-2-amino-4,4-dimethyl-pentoxy]-6-(2,6-dimethylphenyl)pyrimidin-2-yl]sulfamoyl]benzoic acid (hydrochloride salt) (10.16 g, 18.504 mmol) and 1,4-dioxaspiro[4.5]decane-8-carbaldehyde (3.42 g, 19.892 mmol) in DCM (200 mL) was added sodium triacetoxyborohydride (19.21 g, 90.638 mmol). The reaction was stirred for 3 hours and was diluted with 1 N aqueous HCl (200 mL). The reaction was concentrated to concentrate most of the DCM and the aqueous phase was extracted with methyl-THF (3×200 mL). The combined organic layers were dried with sodium sulfate, filtered and concentrated under reduced pressure. The slurry was co-evaporated with heptanes (2×100 mL) and EtOAc (2×100 mL) then the solid was diluted in EtOAc and stirred for 30 minutes. The solid was filtered and dried under reduced pressure to afford 3-[[4-(2,6-dimethylphenyl)-6-[(2R)-2-(1,4-dioxaspiro[4.5]decan-8-ylmethylamino)-4,4-dimethyl-pentoxy]pyrimidin-2-yl]sulfamoyl]benzoic acid (hydrochloride salt) (15.59 g, 86%) as a white solid. ESI-MS m/z calc. 666.3087, found 667.4 (M+1)⁺; Retention time: 1.44 minutes, LC method X.

### Step 3: (11R)-6-(2,6-Dimethylphenyl)-11-(2,2-dimethylpropyl)-12-(1,4-dioxaspiro[4.5]decan-8-ylmethyl)-2,2-dioxo-9-oxa-2λ⁶-thia-3,5,12,19-tetrazatricyclo[12.3.1.14,8]nonadeca-1(18),4(19),5,7,14,16-hexaen-13-one

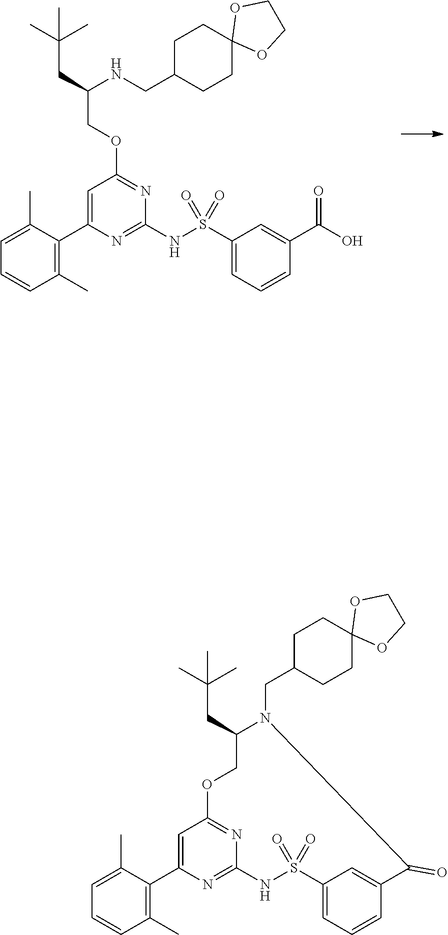 [SMILES: Cc1cccc(C)c1-c1cc(OC[C@@H](CC(C)(C)C)NCC2CCC3(CC2)OCCO3)nc(NS(=O)(=O)c2cccc(C(=O)O)c2)n1 ; Cc1cccc(C)c1-c1cc2nc(n1)NS(=O)(=O)c1cccc(c1)C(=O)N(CC1CCC3(CC1)OCCO3)[C@H](CC(C)(C)C)CO2]

To a solution of 3-[[4-(2,6-dimethylphenyl)-6-[(2R)-2-(1,4-dioxaspiro[4.5]decan-8-ylmethylamino)-4,4-dimethyl-pentoxy]pyrimidin-2-yl]sulfamoyl]benzoic acid (hydrochloride salt) (20.42 g, 24.999 mmol) in dichloromethane (2.6 L) was added N-methylmorpholine (16.560 g, 18 mL, 163.72 mmol) followed by 2-chloro-4,6-dimethoxy-1,3,5-triazine (10.208 g, 58.141 mmol). The reaction was stirred at room temperature for 20 hours. The reaction mixture was concentrated and diluted with 2-methyl-THF (500 mL). The organic layer was washed with an aqueous solution of HCl 1N (2×200 mL) and aqueous solution of NaOH 1M (2×200 mL). The aqueous phase was diluted with brine (100 mL) and back-extracted with 2-methyl-THF (2×200 mL). The combined organic layers were dried over sodium sulfate, filtered and concentrated under reduced pressure, to afford crude (11R)-6-(2,6-dimethylphenyl)-11-(2,2-dimethylpropyl)-12-(1,4-dioxaspiro[4.5]decan-8-ylmethyl)-2,2-dioxo-9-oxa-2λ⁶-thia-3,5,12,19-tetrazatricyclo[12.3.1.14,8]nonadeca-1(18),4(19),5,7,14,16-hexaen-13-one (23.13 g, 91%) as a beige solid. ESI-MS m/z calc. 648.2982, found 649.2 (M+1)⁺; Retention time: 1.92 minutes. LC method X.

### Step 4: (11R)-6-(2,6-Dimethylphenyl)-11-(2,2-dimethylpropyl)-2,2-dioxo-12-[(4-oxocyclohexyl)methyl]-9-oxa-2λ⁶-thia-3,5,12,19-tetrazatricyclo[12.3.1.14,8]nonadeca-1(18),4(19),5,7,14,16-hexaen-13-one

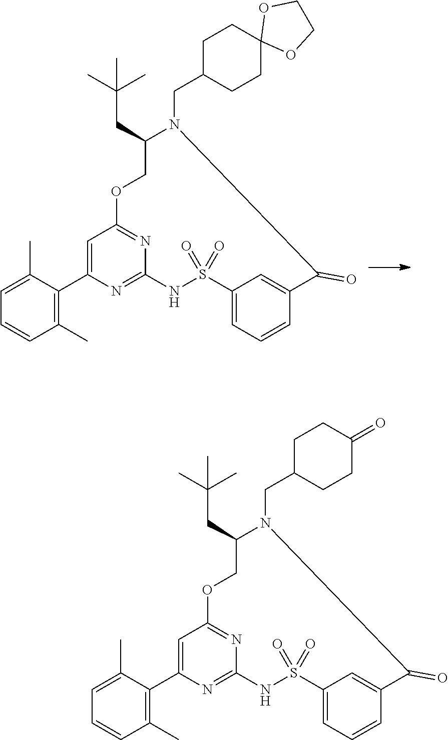 [SMILES: Cc1cccc(C)c1-c1cc2nc(n1)NS(=O)(=O)c1cccc(c1)C(=O)N(CC1CCC3(CC1)OCCO3)[C@H](CC(C)(C)C)CO2 ; Cc1cccc(C)c1-c1cc2nc(n1)NS(=O)(=O)c1cccc(c1)C(=O)N(CC1CCC(=O)CC1)[C@H](CC(C)(C)C)CO2]

To a stirred solution of (11R)-6-(2,6-dimethylphenyl)-11-(2,2-dimethylpropyl)-12-(1,4-dioxaspiro[4.5]decan-8-ylmethyl)-2,2-dioxo-9-oxa-2λ⁶-thia-3,5,12,19-tetrazatricyclo[12.3.1.14,8]nonadeca-1(18),4(19),5,7,14,16-hexaen-13-one (6.35 g, 6.3616 mmol) in acetone (115 mL) and water (15 mL) was added p-toluenesulfonic acid (201 mg, 0.1879 mL, 1.1672 mmol). The reaction mixture was stirred in an oil bath at 65° C. for 5 hours. The solvent was then evaporated, and the aqueous residue was diluted in EtOAc (50 mL). Saturated aqueous KHCO₃ (50 mL) was added and the organic layer was extracted with another portion of saturated aqueous KHCO₃ (50 mL). The combined aqueous phase was washed with EtOAc (3×50 mL). The combined organic layers were washed with brine (50 mL), dried over anhydrous sodium sulfate, filtered and concentrated in vacuo. The residue was purified by chromatography on a 120 g silica gel cartridge with 50 g column using a gradient of 0-100% EtOAc in heptanes then on a reverse phase chromatography on a 275 g C-18 column using a gradient of 50% to 80% MeOH/0.1 0% formic acid water to afford (11R)-6-(2,6-dimethylphenyl)-11-(2,2-dimethylpropyl)-2,2-dioxo-12-[(4-oxocyclohexyl)methyl]-9-oxa-2λ⁶-thia-3,5,12,19-tetrazatricyclo[12.3.1.14,8]nonadeca-1(18),4(19),5,7,14,16-hexaen-13-one (1.41 g, 35%) as a white solid. ¹H NMR (400 MHz, CDCl₃) δ 8.71 (s, 1H), 8.16 (d, J 7.8 Hz, 1H), 7.93 (d, J 7.6 Hz, 1H), 7.69 (t, J 7.8 Hz, 1H), 7.22 (t, J 7.6 Hz, 1H), 7.07 (d, J 7.6 Hz, 2H), 6.25 (s, 1H), 5.31 (dd, J 10.5, 4.2 Hz, 1H), 4.19-4.08 (m, 1H), 3.98-3.90 (m, 1H), 3.90-3.84 (m, 1H), 2.68-2.53 (m, 2H), 2.51-2.33 (m, 4H), 2.33-2.14 (m, 2H), 2.00 (s, 6H), 1.69-1.60 (m, 1H), 1.51-1.36 (m, 3H), 0.57 (s, 9H). ESI-MS m/z calc. 604.2719, found 605.2 (M+1)⁺; Retention time: 4.1 minutes, LC method Y.

### Step 5: (11R)-6-(2,6-dimethylphenyl)-11-(2,2-dimethylpropyl)-12-[(4-hydroxy-4-methyl-cyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 and 2

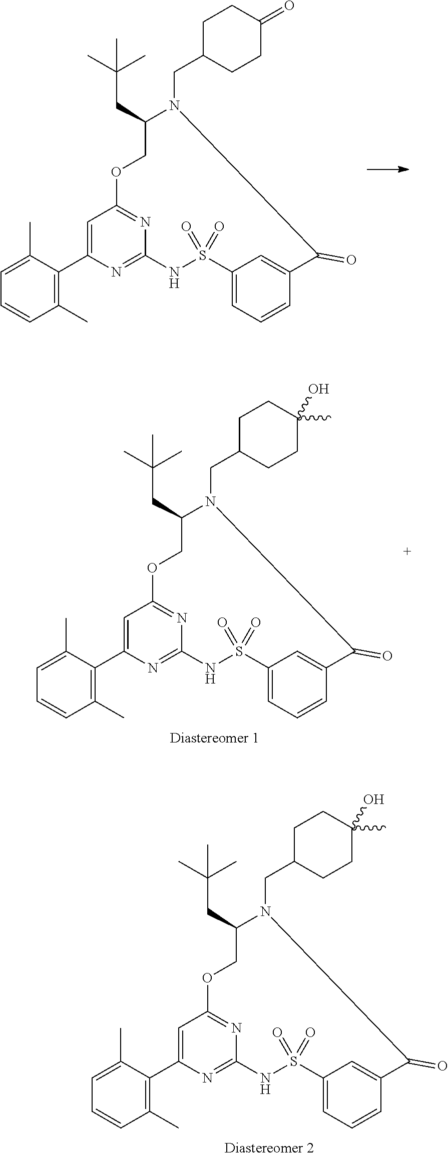

To a solution of (11R)-6-(2,6-dimethylphenyl)-11-(2,2-dimethylpropyl)-2,2-dioxo-12-[(4-oxocyclohexyl)methyl]-9-oxa-2λ⁶-thia-3,5,12,19-tetrazatricyclo[12.3.1.14,8]nonadeca-1(18),4(19),5,7,14,16-hexaen-13-one (178 mg, 0.2799 mmol) in THE (3 mL) was added methyl magnesium bromide solution in diethyl ether (0.5 mL of 3 M, 1.5000 mmol). The reaction was stirred at 0° C. for 30 minutes and the cooling bath was removed. The reaction was stirred at room temperature for 2 hours and the solution was quenched with HCl 1N (20 mL) and diluted with EtOAc (20 mL). The aqueous phase was extracted with EtOAc (3×20 mL) and the combined organic layers were dried over anhydrous sodium sulfate, filtered and concentrated under reduced pressure. The crude residue was purified by silica gel on a 40-g column+25 g snap, eluting from 30% to 100% ethyl acetate in heptanes, to afford (11R)-6-(2,6-dimethylphenyl)-11-(2,2-dimethylpropyl)-12-[(4-hydroxy-4-methyl-cyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (67 mg, 37%), diastereomer 1 and (11R)-6-(2,6-dimethylphenyl)-11-(2,2-dimethylpropyl)-12-[(4-hydroxy-4-methyl-cyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (44 mg, 24%), diastereomer 2 for an overall yield of 61%.

### Step 6: (11R)-6-(2,6-Dimethylphenyl)-11-(2,2-dimethylpropyl)-12-[(4-hydroxy-4-methyl-cyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 149)

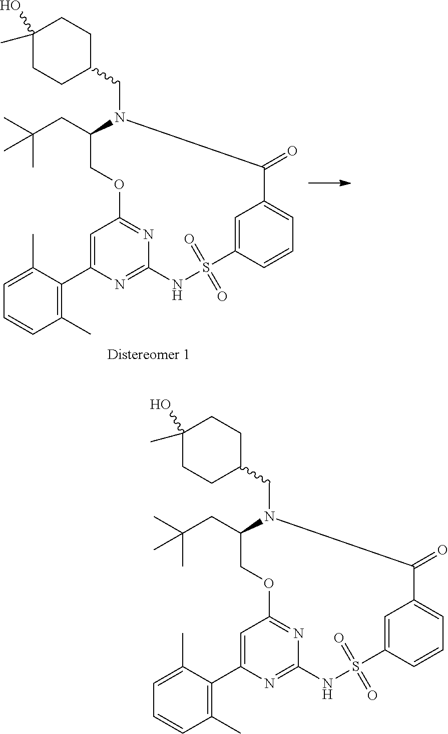 [SMILES: Cc1cccc(C)c1-c1cc2nc(n1)NS(=O)(=O)c1cccc(c1)C(=O)N(CC1CCC(C)(O)CC1)[C@H](CC(C)(C)C)CO2 ; Cc1cccc(C)c1-c1cc2nc(n1)NS(=O)(=O)c1cccc(c1)C(=O)N(CC1CCC(C)(O)CC1)[C@H](CC(C)(C)C)CO2]

Partially purified diastereomer 1 from several experiments were combined (89 mg) and purified by reverse phase chromatography on a 30-g column, eluting from 50% to 80% methanol in water, to afford (11R)-6-(2,6-dimethylphenyl)-11-(2,2-dimethylpropyl)-12-[(4-hydroxy-4-methyl-cyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (53 mg, 65%) as a white solid. ¹H NMR (400 MHz, CDCl₃) δ 8.70 (s, 1H), 8.13 (d, J 7.8 Hz, 1H), 7.91 (d, J 7.8 Hz, 1H), 7.67 (t, J 7.8 Hz, 1H), 7.24-7.19 (m, 1H), 7.07 (d, J 7.6 Hz, 2H), 6.25 (s, 1H), 5.31 (dd, J 10.0, 3.4 Hz, 1H), 4.14-3.96 (m, 2H), 3.75 (dd, J 13.6, 4.3 Hz, 1H), 2.60 (dd, J 13.4, 9.8 Hz, 1H), 2.12-1.94 (m, 7H), 1.80-1.61 (m, 6H), 1.53-1.30 (m, 6H), 1.25 (s, 3H), 0.56 (s, 9H). ESI-MS m/z calc. 620.3032, found 621.3 (M+1)⁺; Retention time: 4.12 minutes; LC method Y.

### Step 7: (11R)-6-(2,6-Dimethylphenyl)-11-(2,2-dimethylpropyl)-12-[(4-hydroxy-4-methyl-cyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 150)

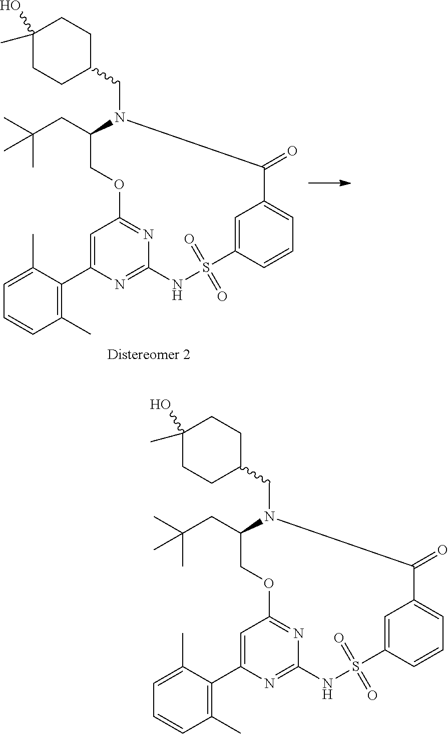 [SMILES: Cc1cccc(C)c1-c1cc2nc(n1)NS(=O)(=O)c1cccc(c1)C(=O)N(CC1CCC(C)(O)CC1)[C@H](CC(C)(C)C)CO2 ; Cc1cccc(C)c1-c1cc2nc(n1)NS(=O)(=O)c1cccc(c1)C(=O)N(CC1CCC(C)(O)CC1)[C@H](CC(C)(C)C)CO2]

Partially purified diastereomer 2 from several experiments were combined (61 mg) and purified by reverse phase chromatography on a 30-g column, eluting from 50% to 80% methanol in water, to afford (11R)-6-(2,6-dimethylphenyl)-11-(2,2-dimethylpropyl)-12-[(4-hydroxy-4-methyl-cyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (39 mg, 72%) as a white solid. ¹H NMR (400 MHz, CDCl₃) δ 8.70 (s, 1H), 8.14 (d, J 7.8 Hz, 1H), 7.92 (d, J 7.6 Hz, 1H), 7.68 (t, J 7.8 Hz, 1H), 7.22 (t, J 7.6 Hz, 1H), 7.08 (d, J 7.6 Hz, 2H), 6.27 (s, 1H), 5.30 (dd, J 10.6, 4.0 Hz, 1H), 4.16-4.07 (m, 1H), 4.03-3.94 (m, 1H), 3.82 (dd, J 13.4, 5.4 Hz, 1H), 2.63 (dd, J 13.8, 8.9 Hz, 1H), 2.02 (br. s., 6H), 1.93-1.74 (m, 4H), 1.66 (dd, J 15.2, 8.3 Hz, 2H), 1.57-1.41 (m, 5H), 1.27 (s, 3H), 1.24-1.05 (m, 2H), 0.56 (s, 9H). ESI-MS m/z calc. 620.3032, found 621.3 (M+1)⁺; Retention time: 4.13 minutes; LC method Y.

### Example 125: Preparation of Compound 151

### Step 1: (11R)-6-(2,6-Dimethylphenyl)-11-(2,2-dimethylpropyl)-12-[(4-hydroxycyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 151)

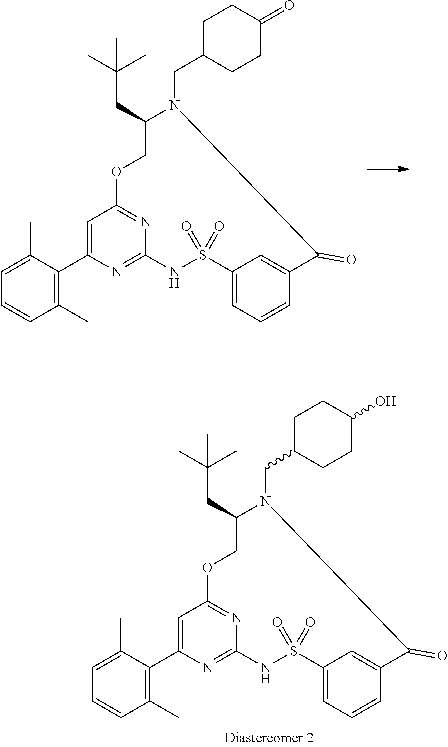 [SMILES: Cc1cccc(C)c1-c1cc2nc(n1)NS(=O)(=O)c1cccc(c1)C(=O)N(CC1CCC(=O)CC1)[C@H](CC(C)(C)C)CO2 ; Cc1cccc(C)c1-c1cc2nc(n1)NS(=O)(=O)c1cccc(c1)C(=O)N(CC1CCC(O)CC1)[C@H](CC(C)(C)C)CO2]

(11R)-6-(2,6-Dimethylphenyl)-11-(2,2-dimethylpropyl)-2,2-dioxo-12-[(4-oxocyclohexyl)methyl]-9-oxa-2λ⁶-thia-3,5,12,19-tetrazatricyclo[12.3.1.14,8]nonadeca-1(18),4(19),5,7,14,16-hexaen-13-one (75 mg, 0.1240 mmol) was dissolved in methanol (0.5 mL). Sodium borohydride (Sodium salt) (6 mg, 0.1586 mmol) was added. The reaction mixture was allowed to stir at room temperature for 1 hour. The reaction mixture was diluted with EtOAc (75 mL). It was then washed with water (1×75 mL) and brine (1×75 mL). The organic layer was dried over sodium sulfate, filtered and concentrated under reduced pressure. The product was isolated by UV-triggered reverse-phase HPLC eluting with a 10-99% acetonitrile/water gradient over 15 minutes with 0.5 mM acid modifier in the aqueous phase. (11R)-6-(2,6-dimethylphenyl)-11-(2,2-dimethylpropyl)-12-[(4-hydroxycyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (16.5 mg, 22%) was obtained as a single isomer. ESI-MS m/z calc. 606.2876, found 607.3 (M+1)⁺; Retention time: 1.67 minutes. LC method A.

### Example 126: Preparation of Compound 152

### Step 1: (11R)-6-(2,6-Dimethylphenyl)-11-(2,2-dimethylpropyl)-12-[(4-hydroxycyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 152)

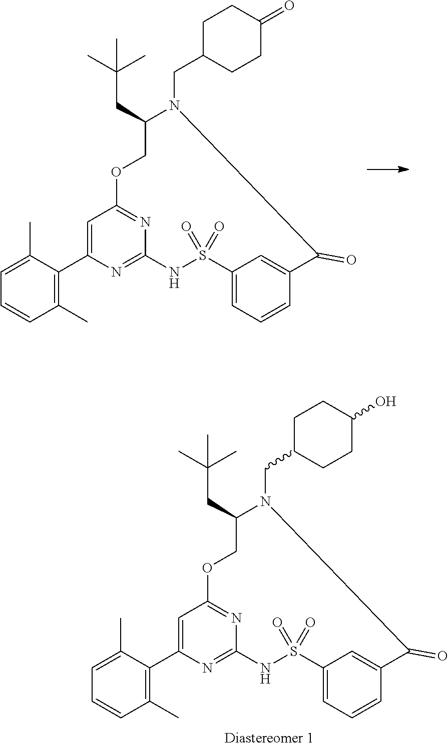 [SMILES: Cc1cccc(C)c1-c1cc2nc(n1)NS(=O)(=O)c1cccc(c1)C(=O)N(CC1CCC(=O)CC1)[C@H](CC(C)(C)C)CO2 ; Cc1cccc(C)c1-c1cc2nc(n1)NS(=O)(=O)c1cccc(c1)C(=O)N(CC1CCC(O)CC1)[C@H](CC(C)(C)C)CO2]

(11R)-6-(2,6-Dimethylphenyl)-11-(2,2-dimethylpropyl)-2,2-dioxo-12-[(4-oxocyclohexyl)methyl]-9-oxa-2λ⁶-thia-3,5,12,19-tetrazatricyclo[12.3.1.14,8]nonadeca-1(18),4(19),5,7,14,16-hexaen-13-one (75 mg, 0.1240 mmol) was dissolved in methanol (0.5 mL). Sodium borohydride (Sodium salt) (7 mg, 0.1850 mmol) was added. The reaction mixture was allowed to stir at room temperature for 1 hour. It was filtered, and the product was isolated by UV-triggered reverse-phase HPLC eluting with a 10-99% water/acetonitrile gradient over 30 minutes with 0.5 mM HCl acid modifier in the aqueous phase. The product was further purified by silica gel column chromatography eluting with a 0-60% EtOAc/hexanes gradient over 50 minutes on a 12-gram silica gel column. (11R)-6-(2,6-dimethylphenyl)-11-(2,2-dimethylpropyl)-12-[(4-hydroxycyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (5.3 mg, 7%) was obtained. ESI-MS m/z calc. 606.2876, found 607.3 (M+1)⁺; Retention time: 1.69 minutes, LC method A.

### Example 127: Preparation of Compound 153

### Step 1: (11R)-6-(2,6-Dimethylphenyl)-11-(2,2-dimethylpropyl)-12-[(4-morpholinocyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 154), and (11R)-6-(2,6-dimethylphenyl)-11-(2,2-dimethylpropyl)-12-[(4-morpholinocyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 153)

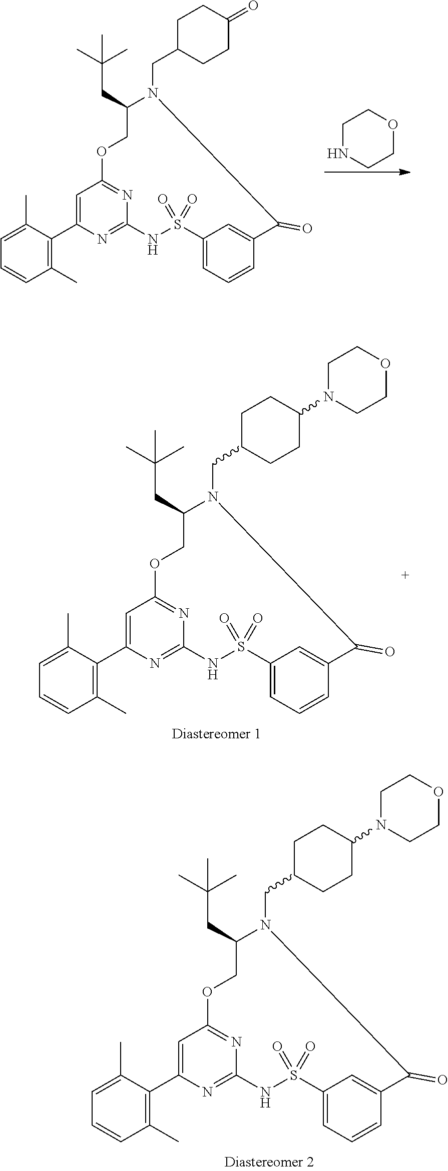 [SMILES: Cc1cccc(C)c1-c1cc2nc(n1)NS(=O)(=O)c1cccc(c1)C(=O)N(CC1CCC(=O)CC1)[C@H](CC(C)(C)C)CO2 ; C1COCCN1 ; Cc1cccc(C)c1-c1cc2nc(n1)NS(=O)(=O)c1cccc(c1)C(=O)N(CC1CCC(N3CCOCC3)CC1)[C@H](CC(C)(C)C)CO2 ; "+" ; Cc1cccc(C)c1-c1cc2nc(n1)NS(=O)(=O)c1cccc(c1)C(=O)N(CC1CCC(N3CCOCC3)CC1)[C@H](CC(C)(C)C)CO2]

(11R)-6-(2,6-Dimethylphenyl)-11-(2,2-dimethylpropyl)-12-[(4-oxocyclohexyl)methyl]-9-oxa-2λ⁶-thia-3,5,12,19-tetraazatricyclo[12.3.1.14,8]nonadeca-1(18),4,6,8(19),14,16-hexaene-2,2,13-trione (50 mg, 0.08268 mmol) was combined with morpholine (14 mg, 0.1607 mmol) and stirred in dichloromethane (0.5 mL) for 30 minutes. Sodium triacetoxyborohydride (35 mg, 0.1651 mmol) was added, and the reaction mixture was allowed to stir overnight. The reaction mixture was filtered and the product was isolated by UV-triggered reverse phase HPLC eluting with a 10-99% acetonitrile/water gradient over 15 minutes with 0.5 mM HCl acid modifier in the aqueous phase, giving two isomers: Diastereomer 1, (11R)-6-(2,6-dimethylphenyl)-11-(2,2-dimethylpropyl)-12-[(4-morpholinocyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (29.0 mg, 52%), ESI-MS m/z calc. 675.34546, found 676.3 (M+1)⁺; Retention time: 1.35 minutes (LC method A); and diastereomer 2, (11R)-6-(2,6-dimethylphenyl)-11-(2,2-dimethylpropyl)-12-[(4-morpholinocyclohexyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (10.6 mg, 19%), ESI-MS m/z calc. 675.34546, found 676.5 (M+1)⁺; Retention time: 1.38 minutes (LC method A).

### Example 128: Preparation of Compound 74

### Step 1: 2-(2-tetrahydropyran-4-ylethylamino)-4-[1-(trifluoromethyl)cyclopropyl]butanenitrile

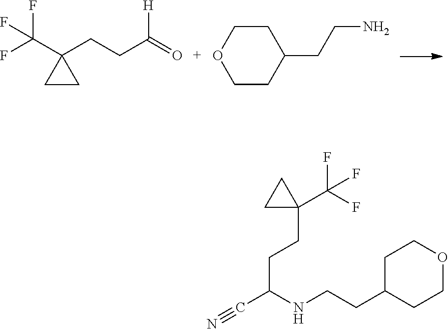

To a stirring solution of 3-[1-(trifluoromethyl)cyclopropyl]propanal (821.9 mg, 4.947 mmol) in acetonitrile (48.19 mL) under nitrogen atmosphere was added 2-tetrahydropyran-4-ylethanamine (639 mg, 4.946 mmol) and trimethylsilylformonitrile (791.4 μL, 5.935 mmol). bromo(dimethyl)sulfonium;bromide (109.8 mg, 0.4947 mmol) was then added and the mixture was stirred for 90 min. Diluted with water (48.19 mL) then removed ˜½ of the acetonitrile by rotary evaporation. Extracted the resulting mixture with EtOAc (3×), combined org. phases, dried (sodium sulfate), filtered and conc. to light tan oil, 2-(2-tetrahydropyran-4-ylethylamino)-4-[1-(trifluoromethyl)cyclopropyl]butanenitrile (1.3 g, 86%) ESI-MS m/z calc. 304.17624, found 305.0 (M+1)⁺; Retention time: 0.39 minutes, and was taken directly to the next step, LC method D.

### Step 2: 2-(2-Tetrahydropyran-4-ylethylamino)-4-[1-(trifluoromethyl)cyclopropyl]butanoic acid

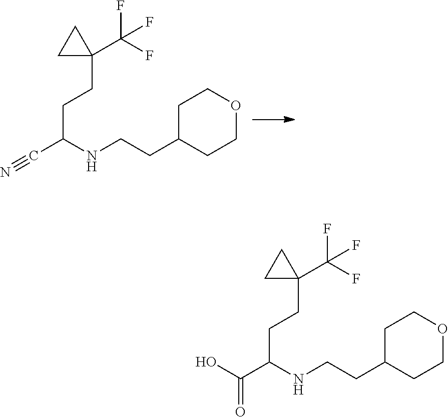 [SMILES: N#CC(CCC1(C(F)(F)F)CC1)NCCC1CCOCC1 ; O=C(O)C(CCC1(C(F)(F)F)CC1)NCCC1CCOCC1]

To a stirring solution of 2-(2-tetrahydropyran-4-ylethylamino)-4-[1-(trifluoromethyl)cyclopropyl]butanenitrile (1.3 g, 4.271 mmol) in acetic acid (813.7 μL, 14.31 mmol) in a vial was added HCl (8.123 mL of 37% w/v, 82.43 mmol) and the vial was capped. The mixture was stirred and heated in an aluminum block at 95° C. for 16 h. The mixture was transferred to a round bottom flask using MeOH and was concentrated by rotary evaporation, including treatment with diethyl ether and removing the solvents three times to give 2-(2-tetrahydropyran-4-ylethylamino)-4-[1-(trifluoromethyl)cyclopropyl]butanoic acid (1.576 g, 100%) ESI-MS m/z calc. 323.17084, found 324.0 (M+1)⁺; Retention time: 0.33 minutes as a light tan solid that was dried thoroughly on the high vacuum pump then taken directly to the next step. LC method D.

### Step 3: 2-(2-Tetrahydropyran-4-ylethylamino)-4-[1-(trifluoromethyl)cyclopropyl]butan-1-ol

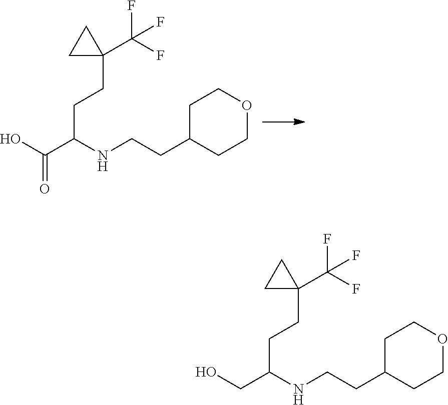 [SMILES: O=C(O)C(CCC1(C(F)(F)F)CC1)NCCC1CCOCC1 ; OCC(CCC1(C(F)(F)F)CC1)NCCC1CCOCC1]

To a stirring solution of 2-(2-tetrahydropyran-4-ylethylamino)-4-[1-(trifluoromethyl)cyclopropyl]butanoic acid (1.576 g, 4.265 mmol) in THE (27.58 mL) under nitrogen atmosphere at 0° C. was slowly added LAH (664.7 mg, 17.06 mmol) and the resulting mixture was stirred at 0° C. for 2 min then allowed to warm to rt and was stirred 75 min. Cooled to 0° C. and quenched by the addition of water (1.279 mL, 71.00 mmol), then KOH (1.279 mL of 15% w/v, 3.419 mmol) then water (2.556 mL, 141.9 mmol). Warmed to rt, added Celite and stirred 5 min then filtered over Celite eluting with ether. The ethereal filtrate was then dried (magnesium sulfate), filtered and concentrated the filtrate by rotary evaporation to an orange oil, 2-(2-tetrahydropyran-4-ylethylamino)-4-[1-(trifluoromethyl)cyclopropyl]butan-1-ol (1.225 g, 93%) ESI-MS m/z calc. 309.19156, found 310.0 (M+1)⁺; Retention time: 0.34 minutes, which was used directly in the next step. LC method D.

### Step 4: 3-[[4-(2,6-Dimethylphenyl)-6-[2-(2-tetrahydropyran-4-ylethylamino)-4-[1-(trifluoromethyl)cyclopropyl]butoxy]pyrimidin-2-yl]sulfamoyl]benzoic acid

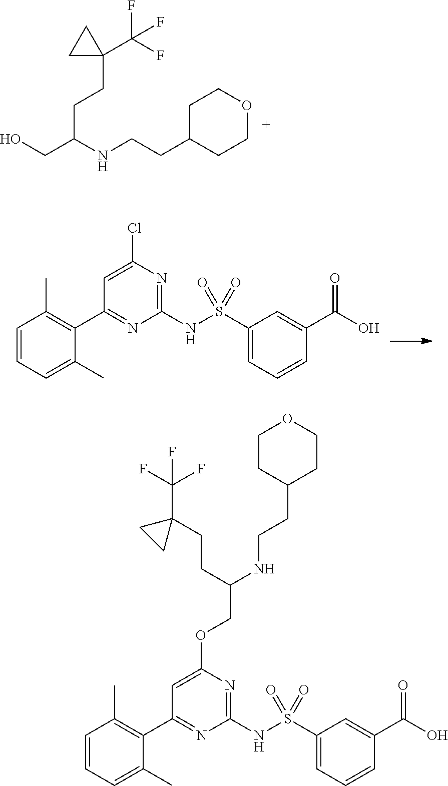

To a stirring solution of 3-[[4-chloro-6-(2,6-dimethylphenyl)pyrimidin-2-yl]sulfamoyl]benzoic acid (675.3 mg, 1.616 mmol) and 2-(2-tetrahydropyran-4-ylethylamino)-4-[1-(trifluoromethyl)cyclopropyl]butan-1-ol (500 mg, 1.616 mmol) in THE (11 mL) at 0° C. was added KOtBu (804.1 μL, 6.464 mmol) and the mixture was stirred at 50° C. for 20 min then removed the acetonitrile by rotary evaporation, dissolved the residue in DMSO, filtered and chromatographed on a 275 g Reverse Phase Column eluting with 20-100% ACN/Water giving 3-[[4-(2,6-dimethylphenyl)-6-[2-(2-tetrahydropyran-4-ylethylamino)-4-[1-(trifluoromethyl)cyclopropyl]butoxy]pyrimidin-2-yl]sulfamoyl]benzoic acid (330 mg, 30%) ESI-MS m/z calc. 690.2699, found 691.1 (M+1)⁺; Retention time: 0.51 minutes LC method D.

### Step 5: 6-(2,6-dimethylphenyl)-2,2-dioxo-12-(2-tetrahydropyran-4-ylethyl)-11-[2-[1-(trifluoromethyl)cyclopropyl]ethyl]-9-oxa-2λ⁶-thia-3,5,12,19-tetrazatricyclo[12.3.1.14,8]nonadeca-1(18),4(19),5,7,14,16-hexaen-13-one (Compound 74)

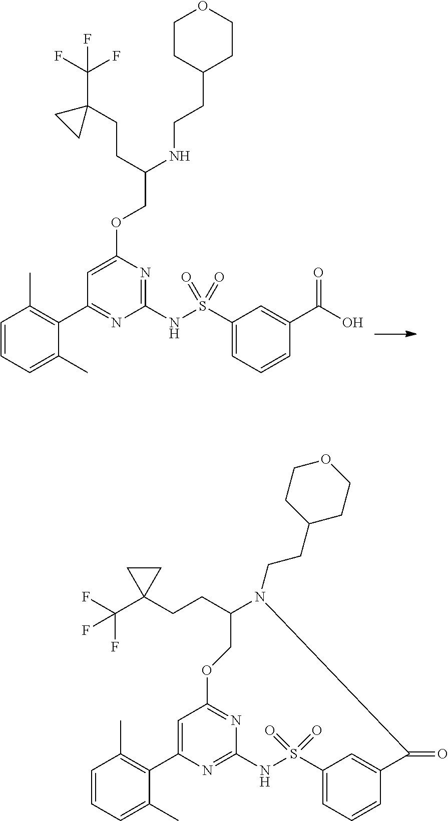 [SMILES: Cc1cccc(C)c1-c1cc(OCC(CCC2(C(F)(F)F)CC2)NCCC2CCOCC2)nc(NS(=O)(=O)c2cccc(C(=O)O)c2)n1 ; Cc1cccc(C)c1-c1cc2nc(n1)NS(=O)(=O)c1cccc(c1)C(=O)N(CCC1CCOCC1)C(CCC1(C(F)(F)F)CC1)CO2]

3-[[4-(2,6-Dimethylphenyl)-6-[2-(2-tetrahydropyran-4-ylethylamino)-4-[1-(trifluoromethyl)cyclopropyl]butoxy]pyrimidin-2-yl]sulfamoyl]benzoic acid (325 mg, 0.4705 mmol) was combined with HATU (232.5 mg, 0.6115 mmol) in DMF (19.5 mL), and DIPEA (246 μL, 1.412 mmol) was added. Stirred at rt overnight then diluted with EtOAc and washed with saturated aqueous ammonium chloride (2×), saturated aqueous sodium bicarbonate (2×) and brine (1×), then dried (magnesium sulfate), filtered and concentrated to an orange oil which was chromatographed on a 275 g Reverse Phase Column eluting with 20-100% ACN/Water giving an impure material that was purified by reverse phase HPLC-MS method using a Luna C₁₈(2) column (75×30 mm, 5 μm particle size) sold by Phenomenex (pn: 00C-4252-U0-AX), and a dual gradient run from 50-99% mobile phase B over 15.0 minutes (mobile phase A=water (5 mM HCl), mobile phase B=acetonitrile, flow rate=50 mL/min, injection volume=950 μL and column temperature=25° C.) giving 6-(2,6-dimethylphenyl)-2,2-dioxo-12-(2-tetrahydropyran-4-ylethyl)-11-[2-[1-(trifluoromethyl)cyclopropyl]ethyl]-9-oxa-2λ⁶-thia-3,5,12,19-tetrazatricyclo[12.3.1.14,8]nonadeca-1(18),4(19),5,7,14,16-hexaen-13-one as a white solid (71.7 mg, 23%). ¹H NMR (400 MHz, Chloroform-d) δ 8.50 (t, J=1.8 Hz, 1H), 8.12 (d, J 7.8 Hz, 1H), 7.74 (dt, J 7.7, 1.4 Hz, 1H), 7.62 (t, J 7.8 Hz, 1H), 7.21 (t, J 7.6 Hz, 1H), 7.00 (d, J=7.6 Hz, 2H), 6.14 (s, 1H), 5.28 (dd, J=10.7, 3.9 Hz, 1H), 4.08-3.93 (m, 3H), 3.93-3.75 (m, 2H), 3.47-3.35 (m, 2H), 2.98 (d, J 2.9 Hz, 1H), 1.92 (d, J 30.5 Hz, 6H), 1.84-1.74 (m, 1H), 1.69 (dt, J 15.0, 7.5 Hz, 4H), 1.53 (d, J 3.8 Hz, 1H), 1.47-1.34 (m, 3H), 0.99 (d, J 3.3 Hz, 1H), 0.95-0.84 (m, 2H), 0.49-0.39 (m, 2H). ESI-MS m/z calc. 672.25934, found 673.1 (M+1)⁺; Retention time: 1.85 minutes (LC method A).

### Example 129: Preparation of Compound 155

### Step 1: (11R)-12-[[5-(2-Azaspiro[3.3]heptan-2-yl)pyrimidin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 155)

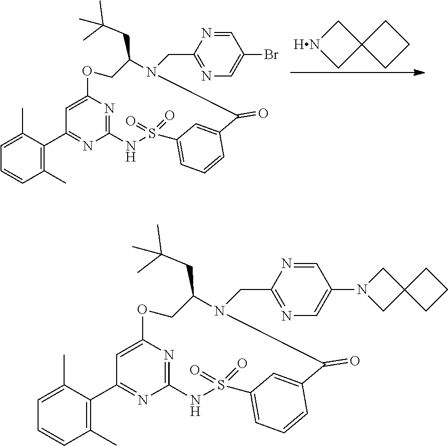

In a tube was introduced (11R)-12-[(5-bromopyrimidin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (30 mg, 0.0451 mmol),2-azaspiro[3.3]heptane (hydrochloride salt) (13 mg, 0.0973 mmol), RuPhos Pd G3 (10.5 mg, 0.0126 mmol) and cesium carbonate (73 mg, 0.2241 mmol). The tube was vacuumed/filled with nitrogen (3 times) before dioxane (0.4 mL) was introduced. The tube was then sealed with a Teflon cap and stirred at 90° C. for 24 h. After cooling down to room temperature, the crude was filtered through a pad of Celite and the pad was rinsed with EtOAc (15 mL). The filtrate was evaporated. The crude material was purified by reverse phase chromatography on 20 g C18 cartridge using a gradient of MeCN in basic water (pH=10) of 30 to 100% for 11 CV then 100% for 2 CV. Fractions containing the product were evaporated and then lyophilized. Afforded (11R)-12-[[5-(2-azaspiro[3.3]heptan-2-yl)pyrimidin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (11.5 mg, 36%) as a white solid. ¹H NMR (400 MHz, DMSO-d₆) δ 12.98 (br. s, 1H), 8.68 (br. s, 1H), 8.02 (s, 2H), 7.90 (d, J 6.8 Hz, 1H), 7.68-7.54 (m, 2H), 7.27-7.18 (m, 1H), 7.14-7.04 (m, 2H), 6.25 (br. s., 1H), 5.40-5.30 (m, 1H), 4.82 (d, J 16.4 Hz, 1H), 4.53 (d, J 16.1 Hz, 1H), 4.14-3.97 (m, 2H), 3.89 (s, 4H), 2.17 (t, J 7.6 Hz, 4H), 2.10-1.88 (m, 6H), 1.85-1.68 (m, 3H), 1.38 (d, J 14.7 Hz, 1H), 0.56 (s, 9H). ESI-MS m/z calc. 681.30975, found 682.4 (M+1)⁺; Retention time: 4.66 minutes; (LC method Y).

### Example 130: Preparation of Compound 156

### Step 1: (11R)-6-(2,6-Dimethylphenyl)-11-(2,2-dimethylpropyl)-12-[[5-(4,4-dimethyltetrahydrofuran-2-yl)-2-pyridyl]methyl]-2,2-dioxo-9-oxa-2λ6-thia-3,5,12,19-tetrazatricyclo[12.3.1.14,8]nonadeca-1(18),4(19),5,7,14,16-hexaen-13-one (Compound 156)

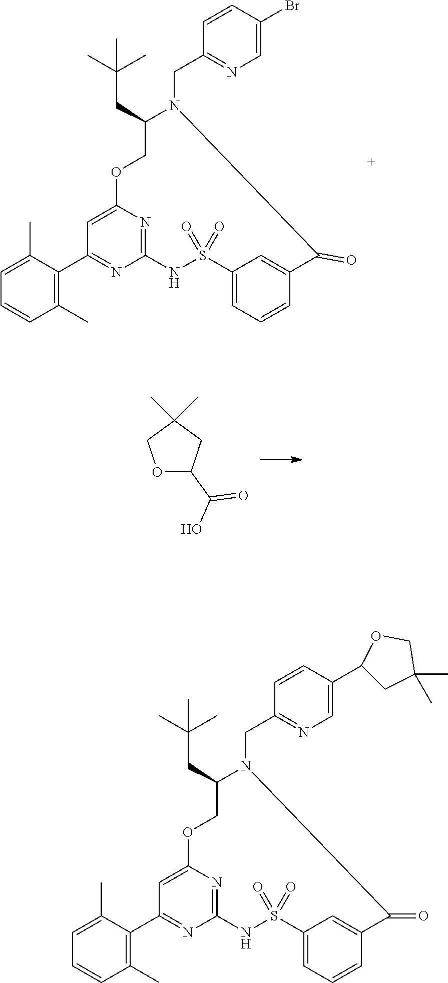

To a 20 mL vial was added (11R)-12-[(5-bromopyridin-2-yl)methyl]-6-(2,6-dimethylphenyl)-11-(2,2-dimethylpropyl)-9-oxa-2λ⁶-thia-3,5,12,19-tetraazatricyclo[12.3.1.14,8]nonadeca-1(17),4(19),5,7,14(18),15-hexaene-2,2,13-trione (70 mg, 0.1053 mmol), 4,4-dimethyltetrahydrofuran-2-carboxylic acid (45.6 mg, 0.3163 mmol), 4-tert-butyl-2-(4-tert-butyl-2-pyridyl)pyridine (4.241 mg, 0.01580 mmol), dichloronickel;1,2-dimethoxyethane (2.314 mg, 0.01053 mmol), (4,4′-Di-tert-butyl-2,2′-bipyridine)bis[(2-pyridinyl)phenyl]iridium(III) (Phosphorus Hexafluoride Ion) (1.181 mg, 0.001053 mmol), and cesium carbonate (103 mg, 0.3161 mmol). The vial was sealed with a Teflon lined septum cap, placed under a nitrogen atmosphere and anhydrous DMF (5 mL) was added. The reaction was degassed by bubbling nitrogen through the reaction solution for 20 minutes. The vial was further sealed by sealing the cap with Parafilm and then irradiated with Kessil blue LEDs with fan running for cooling. After 20 hours, the reaction mixture was diluted with saturated aqueous sodium bicarbonate solution and extracted with ethyl acetate. The combined organics were washed with saturated aqueous sodium chloride solution, dried over sodium sulfate, filtered and concentrated under reduced pressure. The crude material was dissolved in DMF and purified by reverse phase HPLC using (10-99% ACN/H₂O (5 mM HCl)). The desired fractions were combined and partitioned between ethyl acetate and saturated aqueous sodium bicarbonate solution. The organic layer was dried over sodium sulfate, filtered and concentrated under reduced pressure and further dried under high vacuum to provide (11R)-6-(2,6-dimethylphenyl)-11-(2,2-dimethylpropyl)-12-[[5-(4,4-dimethyltetrahydrofuran-2-yl)-2-pyridyl]methyl]-2,2-dioxo-9-oxa-2×6-thia-3,5,12,19-tetrazatricyclo[12.3.1.14,8]nonadeca-1(18),4(19),5,7,14,16-hexaen-13-one (30 mg, 41%) as a white solid. ¹H NMR (400 MHz, DMSO-d₆) δ 12.77 (s, 1H), 8.62 (q, J 1.9 Hz, 1H), 8.51 (t, J 1.7 Hz, 1H), 7.88 (d, J 7.4 Hz, 1H), 7.75 (dt, J 8.1, 2.2 Hz, 1H), 7.58 (d, J 10.6 Hz, 2H), 7.41 (d, J 8.1 Hz, 1H), 7.17 (d, J 7.6 Hz, 1H), 7.07 (d, J 7.6 Hz, 2H), 6.09 (s, 1H), 5.25 (d, J 9.5 Hz, 1H), 5.03 (dd, J 9.4, 6.7 Hz, 1H), 4.84 (d, J 15.7 Hz, 1H), 4.51 (d, J 15.8 Hz, 1H), 4.12 (s, 2H), 3.68 (d, J 7.9 Hz, 1H), 3.55 (d, J 7.9 Hz, 1H), 2.16 (dd, J=12.2, 6.7 Hz, 1H), 1.98 (s, 6H), 1.76 (dd, J=15.1, 8.5 Hz, 1H), 1.61 (dd, J=12.2, 9.5 Hz, 1H), 1.47-1.36 (m, 1H), 1.15 (s, 3H), 1.10 (d, J 1.7 Hz, 3H), 0.53 (s, 9H). ESI-MS m/z calc. 683.31415, found 684.7 (M+1)⁺; Retention time: 1.73 minutes; (LC method A)

### Example 131: Preparation of Compound 157

### Step 1: 3-[[4-[(2R)-2-[(5-Bromo-2-pyridyl)methylamino]-4,4-dimethyl-pentoxy]-6-(2,6-dimethylphenyl)pyrimidin-2-yl]-(methoxymethyl)sulfamoyl]benzoic acid

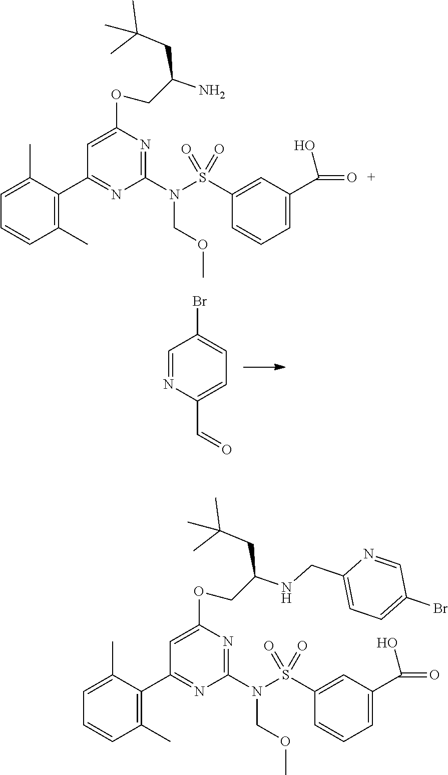

A mixture of 3-[[4-[(2R)-2-amino-4,4-dimethyl-pentoxy]-6-(2,6-dimethylphenyl)pyrimidin-2-yl]-(methoxymethyl)sulfamoyl]benzoic acid (hydrochloride salt) (1.95 g, 3.288 mmol) and 5-bromopyridine-2-carbaldehyde (734 mg, 3.946 mmol) were suspended in anhydrous DCM (10 mL) and stirred at room temperature for 20 minutes. Sodium triacetoxyborohydride (697 mg, 3.289 mmol) was added to the reaction mixture and stirred for 10 minutes followed by the addition of sodium triacetoxyborohydride (2.1 g, 9.908 mmol). The reaction mixture was continued to stir for an additional 30 minutes. The reaction mixture was partitioned between aqueous HCl (20 mL of 1 M, 20.00 mmol), brine and ethyl acetate. The layers were separated, and the aqueous phase was extracted with ethyl acetate (2×). The combined organics were washed with brine, dried over sodium sulfate, and concentrated. The residue was triturated in DCM/hexanes to give a gummy solid. The solvent was decanted out of the flask and the reside was then triturated with Et₂O/hexanes to provide 3-[[4-[(2R)-2-[(5-bromo-2-pyridyl)methylamino]-4,4-dimethyl-pentoxy]-6-(2,6-dimethylphenyl)pyrimidin-2-yl]-(methoxymethyl)sulfamoyl]benzoic acid (hydrochloride salt) (2.45 g, 79%) as an off white solid which contained 19.5% 3-[[4-[(2R)-2-[bis[(5-bromo-2-pyridyl)methyl]amino]-4,4-dimethyl-pentoxy]-6-(2,6-dimethylphenyl)pyrimidin-2-yl]-(methoxymethyl)sulfamoyl]benzoic acid. This material was used directly in the next reaction without any further purification. ESI-MS m z calc. 725.1883, found 726.4 (M+1)⁺; Retention time: 0.63 minutes; (LC method D).

### Step 2: (11R)-12-[(5-Bromo-2-pyridyl)methyl]-6-(2,6-dimethylphenyl)-11-(2,2-dimethylpropyl)-3-(methoxymethyl)-2,2-dioxo-9-oxa-2λ6-thia-3,5,12,19-tetrazatricyclo[12.3.1.14,8]nonadeca-1(18),4(19),5,7,14,16-hexaen-13-one

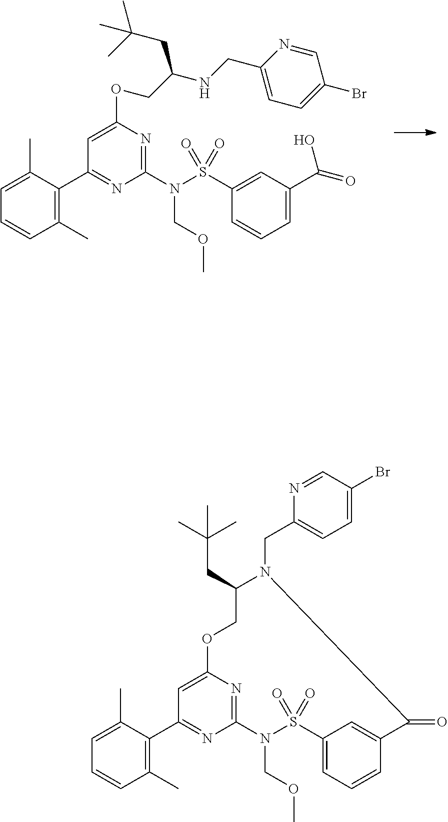 [SMILES: COCN(c1nc(OC[C@@H](CC(C)(C)C)NCc2ccc(Br)cn2)cc(-c2c(C)cccc2C)n1)S(=O)(=O)c1cccc(C(=O)O)c1 ; COCN1c2nc(cc(-c3c(C)cccc3C)n2)OC[C@@H](CC(C)(C)C)N(Cc2ccc(Br)cn2)C(=O)c2cccc(c2)S1(=O)=O]

The crude product from the previous step, 3-[[4-[(2R)-2-[(5-bromo-2-pyridyl)methylamino]-4,4-dimethyl-pentoxy]-6-(2,6-dimethylphenyl)pyrimidin-2-yl]-(methoxymethyl)sulfamoyl]benzoic acid (hydrochloride salt) (2.45 g, 2.584 mmol) was dissolved in anhydrous DMF (200 mL) and cooled in an ice water bath. 2-Chloro-4,6-dimethoxy-1,3,5-triazine (589 mg, 3.355 mmol) was added to the reaction mixture followed by the addition of 4-methylmorpholine (1.7 mL, 15.46 mmol). Stirring was continued with cooling for an additional hour. After 1 hour, the cooling bath was removed, and the reaction was warmed to room temperature and stirred at this temperature for 16 hours. The reaction mixture was partitioned between aqueous HCl (3.4 mL of 1 M, 3.400 mmol), saturated sodium chloride solution and ethyl acetate. The layers were separated, and the aqueous layer was extracted once more with ethyl acetate. The combined organics were then washed with saturated sodium chloride solution three times. The organic layer was dried over sodium sulfate, filtered and concentrated under reduced pressure. The crude product was purified by silica gel flash column chromatography using a gradient of 0-30% ethyl acetate in hexanes to provide (11R)-12-[(5-bromo-2-pyridyl)methyl]-6-(2,6-dimethylphenyl)-11-(2,2-dimethylpropyl)-3-(methoxymethyl)-2,2-dioxo-9-oxa-2λ6-thia-3,5,12,19-tetrazatricyclo[12.3.1.14,8]nonadeca-1(18),4(19),5,7,14,16-hexaen-13-one (602 mg, 33%) as a white solid. ¹H NMR (400 MHz, DMSO-d₆) δ 8.77 (d, J 1.9 Hz, 1H), 8.68 (dd, J 2.4, 0.7 Hz, 1H), 8.16 (dt, J 7.1, 1.9 Hz, 1H), 8.05 (dd, J 8.4, 2.4 Hz, 1H), 7.88-7.75 (m, 2H), 7.49 (d, J 8.4 Hz, 1H), 7.27-7.17 (m, 1H), 7.11 (d, J 7.6 Hz, 2H), 6.72 (s, 1H), 5.74 (d, J 11.0 Hz, 1H), 5.56 (d, J 11.0 Hz, 1H), 5.21 (dd, J 11.1, 4.5 Hz, 1H), 4.82 (d, J 16.0 Hz, 1H), 4.61 (d, J 16.0 Hz, 1H), 4.28 (t, J 11.4 Hz, 1H), 4.18-4.08 (m, 1H), 2.96 (s, 3H), 1.96 (s, 6H), 1.80 (dd, J 15.3, 8.7 Hz, 1H), 1.50-1.38 (m, 1H), 0.51 (s, 9H). ESI-MS m/z calc. 707.1777, found 708.38 (M+1)⁺; Retention time: 0.9 minutes; (LC method D).

### Step 3: (11R)-6-(2,6-dimethylphenyl)-11-(2,2-dimethylpropyl)-12-[[5-(2,2-dimethylspiro[3.3]heptan-6-yl)-2-pyridyl]methyl]-3-(methoxymethyl)-2,2-dioxo-9-oxa-2%6-thia-3,5,12,19-tetrazatricyclo[12.3.1.14,8]nonadeca-1(18),4(19),5,7,14,16-hexaen-13-one

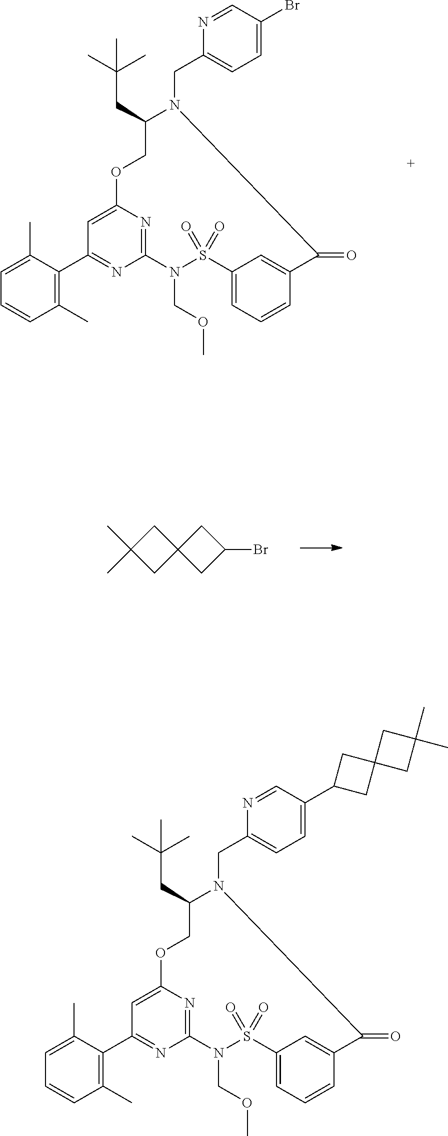

Solution 1: Anhydrous DME (2 mL) was added to a mixture of dichloronickel;1,2-dimethoxyethane (0.31 mg, 0.001411 mmol) and 4-tert-butyl-2-(4-tert-butyl-2-pyridyl)pyridine (0.38 mg, 0.001416 mmol) under a nitrogen atmosphere and stirred for 15 minutes. In a 20 mL vial was added (11R)-12-[(5-bromo-2-pyridyl)methyl]-6-(2,6-dimethylphenyl)-11-(2,2-dimethylpropyl)-3-(methoxymethyl)-2,2-dioxo-9-oxa-2×6-thia-3,5,12,19-tetrazatricyclo[12.3.1.14,8]nonadeca-1(18),4(19),5,7,14,16-hexaen-13-one (100 mg, 0.1411 mmol), 6-bromo-2,2-dimethyl-spiro[3.3]heptane (45.3 mg, 0.2119 mmol), Tris(trimethylsilyl)silane (44.9 μL, 0.1455 mmol), sodium carbonate (30 mg, 0.2830 mmol) and (4,4′-Di-tert-butyl-2,2′-bipyridine)bis[(2-pyridinyl)phenyl]iridium(III) (Phosphorus Hexafluoride Ion) (1.583 mg, 0.001411 mmol). The vial was sealed with a Teflon lined septum cap and placed under a nitrogen atmosphere followed by the addition of anhydrous DME (3.7 mL). 1 mL (0.141 mol % catalyst, 0.706 mol, 0.005 equivalent) of solution 1 was then syringed into this reaction mixture, which was then degassed by sparging with nitrogen while stirring for 20 minutes. The vial was further sealed by sealing the cap with Parafilm and then irradiated with Kessil blue LEDs with fan running for cooling. After 5 hours, the reaction mixture was quenched by exposure to air, filtered through a pad of Celite and the filtrate was concentrated under reduced pressure. The crude material was purified by silica gel column chromatography using a gradient of 0-30% ethyl acetate in hexanes to provide (11R)-6-(2,6-dimethylphenyl)-11-(2,2-dimethylpropyl)-12-[[5-(2,2-dimethylspiro[3.3]heptan-6-yl)-2-pyridyl]methyl]-3-(methoxymethyl)-2,2-dioxo-9-oxa-2λ6-thia-3,5,12,19-tetrazatricyclo[12.3.1.14,8]nonadeca-1(18),4(19),5,7,14,16-hexaen-13-one (36 mg, 34%) as a white solid. ESI-MS m/z calc. 751.3768, found 752.7 (M+1)⁺; Retention time: 0.88 minutes; (LC method D).

### Step 4: (11R)-6-(2,6-Dimethylphenyl)-11-(2,2-dimethylpropyl)-12-[[5-(2,2-dimethylspiro[3.3]heptan-6-yl)-2-pyridyl]methyl]-2,2-dioxo-9-oxa-2λ6-thia-3,5,12,19-tetrazatricyclo[12.3.1.14,8]nonadeca-1(18),4(19),5,7,14,16-hexaen-13-one (Compound 157)

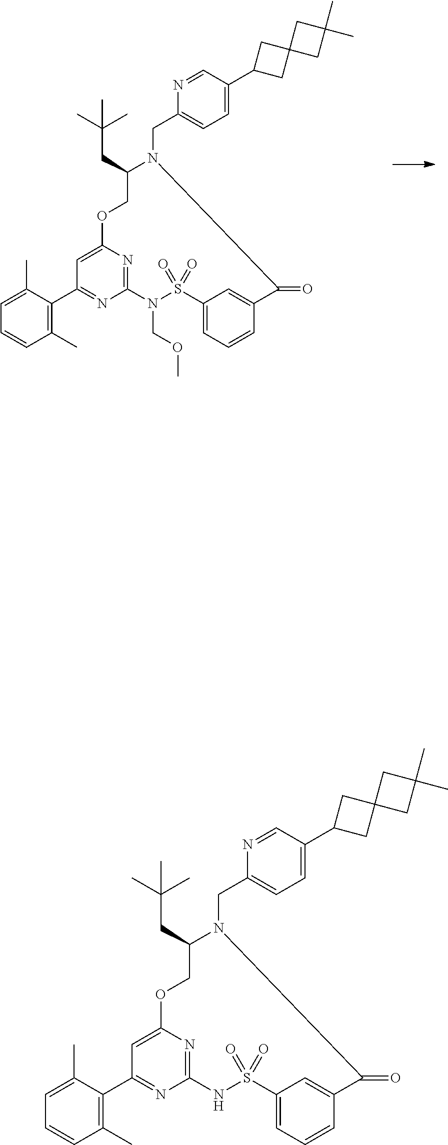 [SMILES: COCN1c2nc(cc(-c3c(C)cccc3C)n2)OC[C@@H](CC(C)(C)C)N(Cc2ccc(C3CC4(C3)CC(C)(C)C4)cn2)C(=O)c2cccc(c2)S1(=O)=O ; Cc1cccc(C)c1-c1cc2nc(n1)NS(=O)(=O)c1cccc(c1)C(=O)N(Cc1ccc(C3CC4(C3)CC(C)(C)C4)cn1)[C@H](CC(C)(C)C)CO2]

(11R)-6-(2,6-Dimethylphenyl)-11-(2,2-dimethylpropyl)-12-[[5-(2,2-dimethylspiro[3.3]heptan-6-yl)-2-pyridyl]methyl]-3-(methoxymethyl)-2,2-dioxo-9-oxa-26-thia-3,5,12,19-tetrazatricyclo[12.3.1.14,8]nonadeca-1(18),4(19),5,7,14,16-hexaen-13-one (36 mg, 0.04787 mmol) was dissolved in DCM (360 μL) followed by the addition of TFA (74 μL, 0.9605 mmol) and stirred at room temperature for 40 minutes. The reaction mixture was diluted with DMSO to 1 mL, syringe filtered and purified by reverse phase HPLC using (10-99% ACN/H₂O (5 mM HCl)) to provide (11R)-6-(2,6-dimethylphenyl)-11-(2,2-dimethylpropyl)-12-[[5-(2,2-dimethylspiro[3.3]heptan-6-yl)-2-pyridyl]methyl]-2,2-dioxo-9-oxa-2λ6-thia-3,5,12,19-tetrazatricyclo[12.3.1.14,8]nonadeca-1(18),4(19),5,7,14,16-hexaen-13-one (24.8 mg, 72%) as a white solid. ESI-MS m/z calc. 707.3505, found 708.7 (M+1)⁺; Retention time: 1.98 minutes (LC method A).

### Example 132: Preparation of (3R,7R)-19-(2,6-Dimethylphenyl)-5-{spiro[3.5]nonan-2-yl}-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (Compound 1190)

### Step 1: 3-[[4-[(3R,4R)-4-Amino-1-tert-butoxycarbonyl-pyrrolidin-3-yl]oxy-6-(2,6-dimethylphenyl)pyrimidin-2-yl]sulfamoyl]benzoic acid

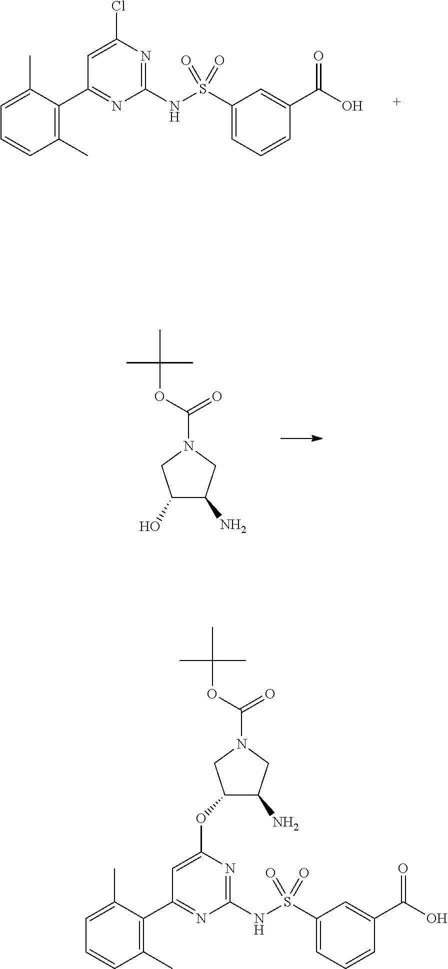 [SMILES: Cc1cccc(C)c1-c1cc(Cl)nc(NS(=O)(=O)c2cccc(C(=O)O)c2)n1 ; CC(C)(C)OC(=O)N1C[C@@H](N)[C@H](O)C1 ; Cc1cccc(C)c1-c1cc(O[C@@H]2CN(C(=O)OC(C)(C)C)C[C@H]2N)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (10.6 g, 25.37 mmol), tert-butyl (3R,4R)-3-amino-4-hydroxy-pyrrolidine-1-carboxylate (5.2 g, 25.71 mmol), and sodium t-butoxide (7.3 g, 75.96 mmol) in THE (0.13 L) was stirred for 18 hours. The reaction was acidified with 1 M citric acid, diluted with water, and extracted with ethyl acetate. The combined extracts were washed with brine, dried over sodium sulfate, and evaporated under vacuum to give a tan oil. The oil was stirred with diethyl ether to give a colorless solid. The solid was filtered, washed with diethyl ether, and dried under vacuum to give 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (15.2 g, 103%) ESI-MS m/z calc. 583.2101, found 584.3 (M+1)⁺; Retention time: 0.49 minutes as a colorless solid, LC method D.

### Step 2: 3-[[4-[(3R,4R)-1-tert-Butoxycarbonyl-4-[2-[1-(trifluoromethyl)cyclopropyl]ethylamino]pyrrolidin-3-yl]oxy-6-(2,6-dimethylphenyl)pyrimidin-2-yl]sulfamoyl]benzoic acid

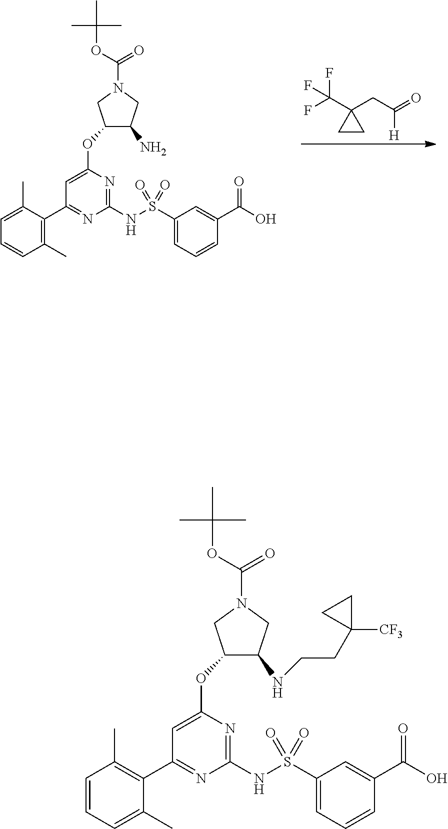 [SMILES: Cc1cccc(C)c1-c1cc(O[C@@H]2CN(C(=O)OC(C)(C)C)C[C@H]2N)nc(NS(=O)(=O)c2cccc(C(=O)O)c2)n1 ; O=CCC1(C(F)(F)F)CC1 ; Cc1cccc(C)c1-c1cc(O[C@@H]2CN(C(=O)OC(C)(C)C)C[C@H]2NCCC2(C(F)(F)F)CC2)nc(NS(=O)(=O)c2cccc(C(=O)O)c2)n1]

A solution of 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (291.8 mg, 0.5 mmol), 2-[1-(trifluoromethyl)cyclopropyl]acetaldehyde (approximately 83.66 mg, 0.5500 mmol), and sodium triacetoxyborohydride (approximately 211.9 mg, 1.000 mmol) in dichloromethane (2.500 mL) was stirred for 18 hours. More 2-[1-(trifluoromethyl)cyclopropyl]acetaldehyde (approximately 83.66 mg, 0.5500 mmol) and sodium triacetoxyborohydride (approximately 211.9 mg, 1.000 mmol) were again added, and the reactions were stirred for 22 hours. The solvent was evaporated, and the residue was diluted with water, acidified with 1 M citric acid, and extracted with ethyl acetate. The residue was purified by silica gel column chromatography with 0-8% methanol in dichloromethane to give 3-[[4-[(3R,4R)-1-tert-butoxycarbonyl-4-[2-[1-(trifluoromethyl)cyclopropyl]ethylamino]pyrrolidin-3-yl]oxy-6-(2,6-dimethylphenyl)pyrimidin-2-yl]sulfamoyl]benzoic acid (0.19 g, 53%). ESI-MS m/z calc. 719.2601, found 720.3 (M+1)⁺; Retention time: 0.57 minutes; LC method D.

### Step 3: tert-Butyl (3R,7R)-19-(2,6-dimethylphenyl)-9,15,15-trioxo-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-5-carboxylate

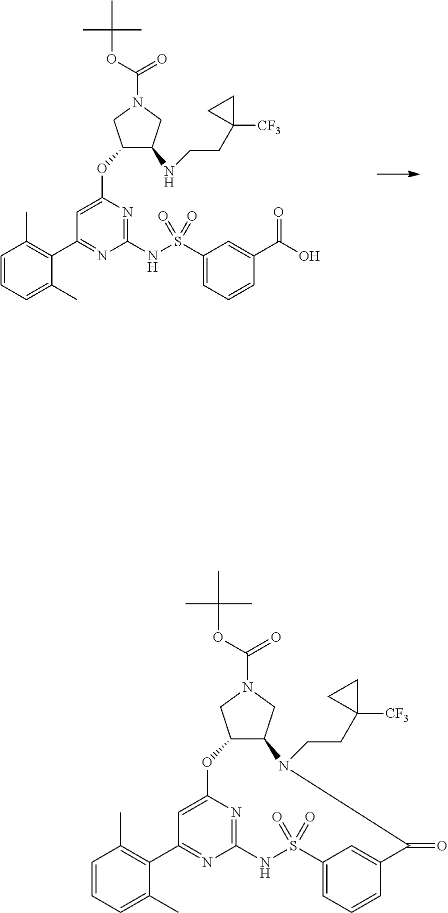 [SMILES: Cc1cccc(C)c1-c1cc(O[C@@H]2CN(C(=O)OC(C)(C)C)C[C@H]2NCCC2(C(F)(F)F)CC2)nc(NS(=O)(=O)c2cccc(C(=O)O)c2)n1 ; Cc1cccc(C)c1-c1cc2nc(n1)NS(=O)(=O)c1cccc(c1)C(=O)N(CCC1(C(F)(F)F)CC1)[C@@H]1CN(C(=O)OC(C)(C)C)C[C@H]1O2]

A solution of 3-[[4-[(3R,4R)-1-tert-butoxycarbonyl-4-[2-[1-(trifluoromethyl)cyclopropyl]ethylamino]pyrrolidin-3-yl]oxy-6-(2,6-dimethylphenyl)pyrimidin-2-yl]sulfamoyl]benzoic acid (0.19 g, 0.2640 mmol), [[(E)-(1-cyano-2-ethoxy-2-oxo-ethylidene)amino]oxy-tetrahydropyran-4-yl-methylene]-dimethyl-ammonium (Phosphorus Hexafluoride Ion) (0.17 g, 0.3979 mmol), and DIEA (0.14 mL, 0.8038 mmol) in DMF (25 mL) was stirred for three days. The reaction was acidified with 1 M citric acid, diluted with water, and extracted with ethyl acetate. The combined extracts were washed with brine, dried over sodium sulfate, and evaporated under vacuum. The residue was purified by silica gel column chromatography with 0-8% methanol in dichloromethane to give tert-butyl (3R,7R)-19-(2,6-dimethylphenyl)-9,15,15-trioxo-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-5-carboxylate (0.17 g, 92%) as an orange solid. ESI-MS m/z calc. 701.2495, found 702.3 (M+1)⁺; Retention time: 0.75 minutes, LC method D.

### Step 4: (3R,7R)-19-(2,6-Dimethylphenyl)-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione

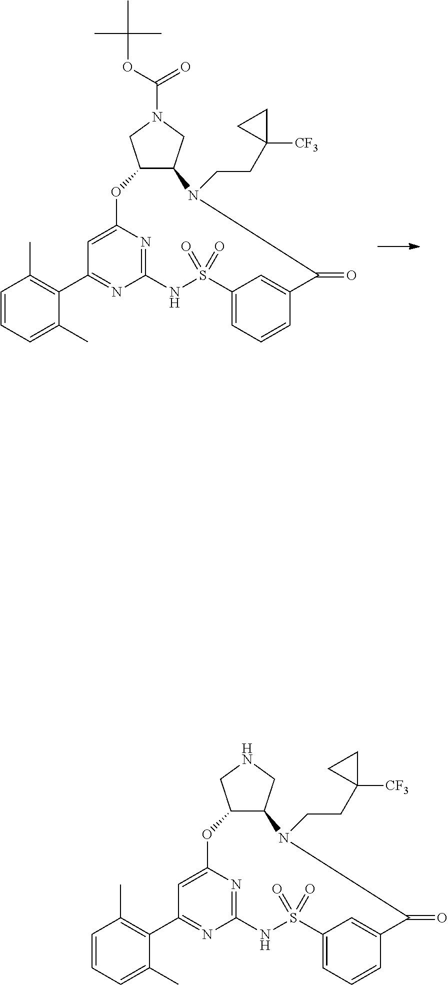 [SMILES: Cc1cccc(C)c1-c1cc2nc(n1)NS(=O)(=O)c1cccc(c1)C(=O)N(CCC1(C(F)(F)F)CC1)[C@@H]1CN(C(=O)OC(C)(C)C)C[C@H]1O2 ; Cc1cccc(C)c1-c1cc2nc(n1)NS(=O)(=O)c1cccc(c1)C(=O)N(CCC1(C(F)(F)F)CC1)[C@@H]1CNC[C@H]1O2]

A solution of tert-butyl (3R,7R)-19-(2,6-dimethylphenyl)-9,15,15-trioxo-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-5-carboxylate (0.17 g, 0.2422 mmol) in HCl (4 mL of 4 M, 16.00 mmol) (in dioxane) was stirred for 30 minutes, and the solvent was removed under vacuum. The solids were triturated with diethyl ether and dried under vacuum to give (3R,7R)-19-(2,6-dimethylphenyl)-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (0.14 g, 91%) as a colorless solid. ESI-MS m/z calc. 601.1971, found 602.3 (M+1)⁺; Retention time: 0.4 minutes, LC method D.

### Step 5: (3R,7R)-19-(2,6-Dimethylphenyl)-5-{spiro[3.5]nonan-2-yl}-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (Compound 1190)

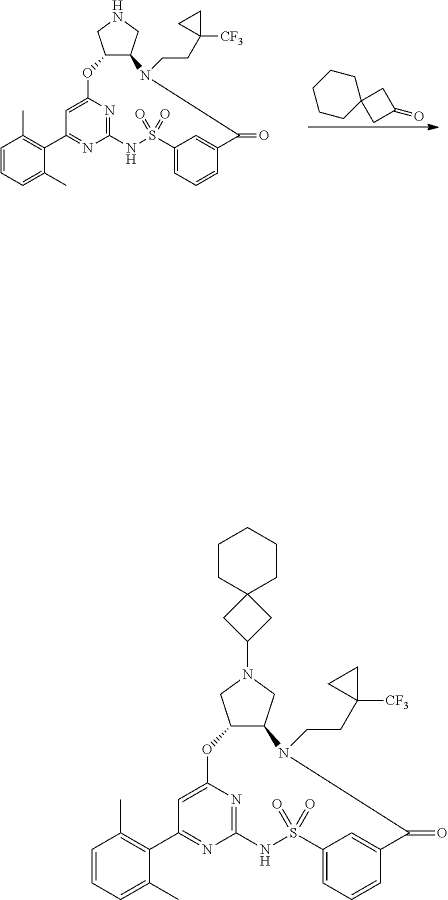 [SMILES: Cc1cccc(C)c1-c1cc2nc(n1)NS(=O)(=O)c1cccc(c1)C(=O)N(CCC1(C(F)(F)F)CC1)[C@@H]1CNC[C@H]1O2 ; O=C1CC2(CCCCC2)C1 ; Cc1cccc(C)c1-c1cc2nc(n1)NS(=O)(=O)c1cccc(c1)C(=O)N(CCC1(C(F)(F)F)CC1)[C@@H]1CN(C3CC4(CCCCC4)C3)C[C@H]1O2]

A solution of (3R,7R)-19-(2,6-dimethylphenyl)-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (40 mg, 0.06269 mmol), spiro[3.5]nonan-2-one (18 mg, 0.1302 mmol), and sodium triacetoxyborohydride (41 mg, 0.1935 mmol) in dichloromethane (0.3 mL) was stirred for four hours. More spiro[3.5]nonan-2-one (18 mg, 0.1302 mmol) and sodium triacetoxyborohydride (41 mg, 0.1935 mmol) were added, and the reaction was stirred for four days. The reaction was stirred with methanol, and the solvents were removed under vacuum. The residue was purified by reverse-phase HPLC-MS (1%-99% acetonitrile/water (5 mM HCl)) to give (3R,7R)-19-(2,6-dimethylphenyl)-5-{spiro[3.5]nonan-2-yl}-8-{2-[1-(trifluoromethyl)cyclopropyl]ethyl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (17.8 mg, 37%) as a light yellow solid. ESI-MS m/z calc. 723.30664, found 724.5 (M+1)⁺; Retention time: 1.61 minutes, LC method A.

### Example 133: Preparation of (3R,7R)-19-(2,6-Dimethylphenyl)-8-[2-(oxan-4-yl)ethyl]-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (Compound 1191)

### Step 1: 3-[[4-[(3R,4R)-1-tert-Butoxycarbonyl-4-(2-tetrahydropyran-4-ylethylamino)pyrrolidin-3-yl]oxy-6-(2,6-dimethylphenyl)pyrimidin-2-yl]sulfamoyl]benzoic acid

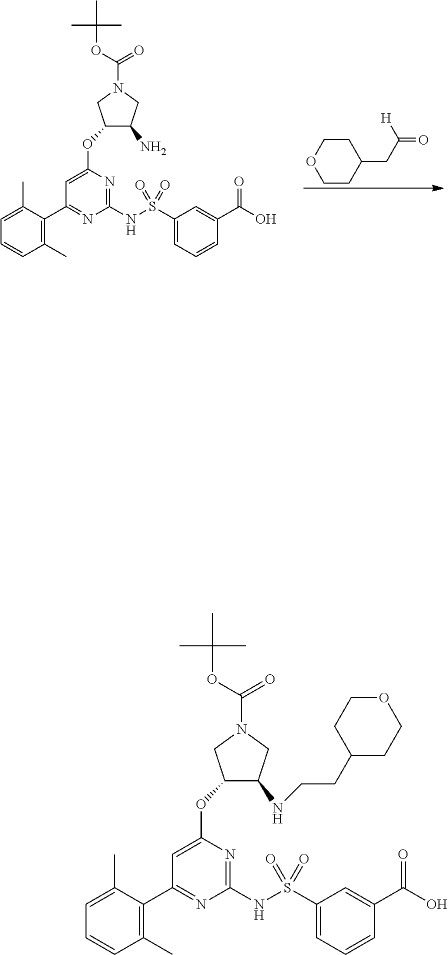 [SMILES: Cc1cccc(C)c1-c1cc(O[C@@H]2CN(C(=O)OC(C)(C)C)C[C@H]2N)nc(NS(=O)(=O)c2cccc(C(=O)O)c2)n1 ; O=CCC1CCOCC1 ; Cc1cccc(C)c1-c1cc(O[C@@H]2CN(C(=O)OC(C)(C)C)C[C@H]2NCCC2CCOCC2)nc(NS(=O)(=O)c2cccc(C(=O)O)c2)n1]

A solution of 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (0.12 g, 0.2056 mmol), 2-tetrahydropyran-4-ylacetaldehyde (52 mg, 0.4057 mmol), and sodium triacetoxyborohydride (0.13 g, 0.6134 mmol) in dichloromethane (1 mL) was stirred for 19 hours. The reaction was stirred with methanol, the volatiles were removed under vacuum, and the residue was purified by reverse-phase HPLC-MS (1%-99% acetonitrile/water (5 mM HCl)) to give a mixture containing product and deprotected product. The mixture was re-purified by reverse-phase HPLC-MS (1%-99% acetonitrile/water) to give 3-[[4-[(3R,4R)-1-tert-butoxycarbonyl-4-(2-tetrahydropyran-4-ylethylamino)pyrrolidin-3-yl]oxy-6-(2,6-dimethylphenyl)pyrimidin-2-yl]sulfamoyl]benzoic acid (16 mg, 11%) as a colorless solid. ESI-MS m/z calc. 695.2989, found 696.4 (M+1)⁺; Retention time: 0.52 minutes, LC method D.

### Step 2: (3R,7R)-19-(2,6-dimethylphenyl)-8-[2-(oxan-4-yl)ethyl]-2-oxa-15λ⁶-thia-5,8,16,18,21-pentaazatetracyclo[15.3.1.110,14.03,7]docosa-1(20),10(22),11,13,17(21),18-hexaene-9,15,15-trione

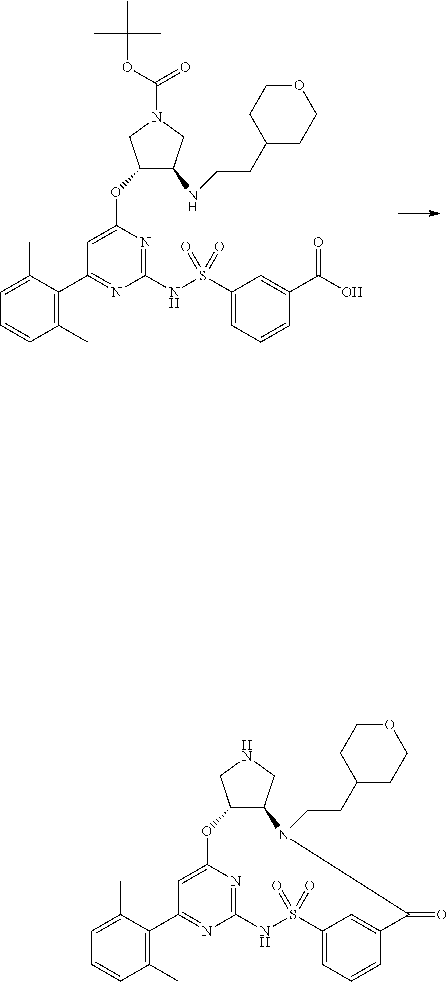 [SMILES: Cc1cccc(C)c1-c1cc(O[C@@H]2CN(C(=O)OC(C)(C)C)C[C@H]2NCCC2CCOCC2)nc(NS(=O)(=O)c2cccc(C(=O)O)c2)n1 ; Cc1cccc(C)c1-c1cc2nc(n1)NS(=O)(=O)c1cccc(c1)C(=O)N(CCC1CCOCC1)[C@@H]1CNC[C@H]1O2]

A solution of 3-[[4-[(3R,4R)-1-tert-butoxycarbonyl-4-(2-tetrahydropyran-4-ylethylamino)pyrrolidin-3-yl]oxy-6-(2,6-dimethylphenyl)pyrimidin-2-yl]sulfamoyl]benzoic acid (16 mg, 0.02299 mmol), HATU (14 mg, 0.03682 mmol), and DIEA (13 μL, 0.07463 mmol) in DMF (2 mL) was stirred for 17 hours. The reaction was diluted with water, acidified with 1 M citric acid, and extracted with ethyl acetate. The combined extracts were washed with brine, dried over sodium sulfate, and evaporated under vacuum. The residue was stirred with HCl (3 mL of 4 M, 12.00 mmol) (in dioxane) for one hour. The solvent was evaporated under vacuum, and the residue was purified by reverse-phase HPLC-MS (1%-99% acetonitrile/water (5 mM HCl)) to give (3R,7R)-19-(2,6-dimethylphenyl)-8-[2-(oxan-4-yl)ethyl]-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (7 mg, 50%) as a colorless solid. ESI-MS m/z calc. 577.2359, found 578.3 (M+1)⁺; Retention time: 0.34 minutes, LC method D.

### Step 3: (3R,7R)-19-(2,6-Dimethylphenyl)-8-[2-(oxan-4-yl)ethyl]-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (Compound 1191)

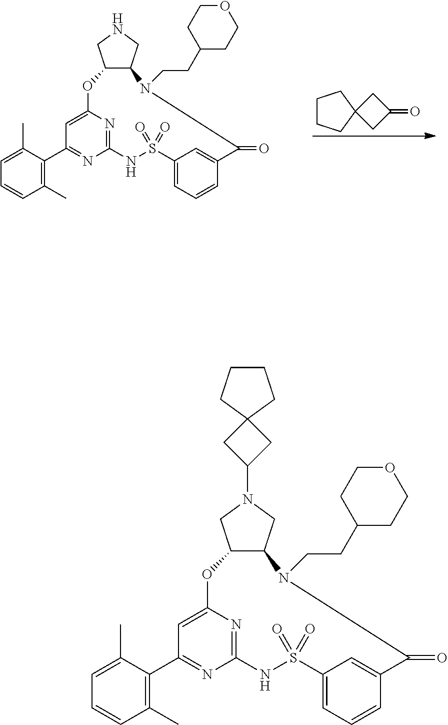 [SMILES: Cc1cccc(C)c1-c1cc2nc(n1)NS(=O)(=O)c1cccc(c1)C(=O)N(CCC1CCOCC1)[C@@H]1CNC[C@H]1O2 ; O=C1CC2(CCCC2)C1 ; Cc1cccc(C)c1-c1cc2nc(n1)NS(=O)(=O)c1cccc(c1)C(=O)N(CCC1CCOCC1)[C@@H]1CN(C3CC4(CCCC4)C3)C[C@H]1O2]

A solution of (3R,7R)-19-(2,6-dimethylphenyl)-8-[2-(oxan-4-yl)ethyl]-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (7 mg, 0.01140 mmol), spiro[3.4]octan-2-one (5 μL, 0.04026 mmol), and sodium triacetoxyborohydride (10 mg, 0.04718 mmol) in dichloromethane (0.2 mL) was stirred for two hours. The reaction was stirred with methanol, the volatiles were removed under vacuum, and the residue was purified by reverse-phase HPLC-MS (20%-80% acetonitrile/water (5 mM HCl)) to give (3R,7R)-19-(2,6-dimethylphenyl)-8-[2-(oxan-4-yl)ethyl]-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (7 mg, 84%) as a colorless solid. ESI-MS m/z calc. 685.3298, found 686.5 (M+1)⁺; Retention time: 1.33 minutes, LC method A.

### Example 134: Preparation of (3R,7R)-19-(2,6-Dimethylphenyl)-8-ethyl-5-{spiro[3.4]octan-2-yl}-2-oxa-15λ⁶-thia-5,8,16,18,21-pentaazatetracyclo[15.3.1.110,14.03,7]docosa-1(20),10(22),11,13,17(21),18-hexaene-9,15,15-trione (Compound 1192)

### Step 1: 3-[[4-[(3R,4R)-1-tert-Butoxycarbonyl-4-(ethylamino)pyrrolidin-3-yl]oxy-6-(2,6-dimethylphenyl)pyrimidin-2-yl]sulfamoyl]benzoic acid

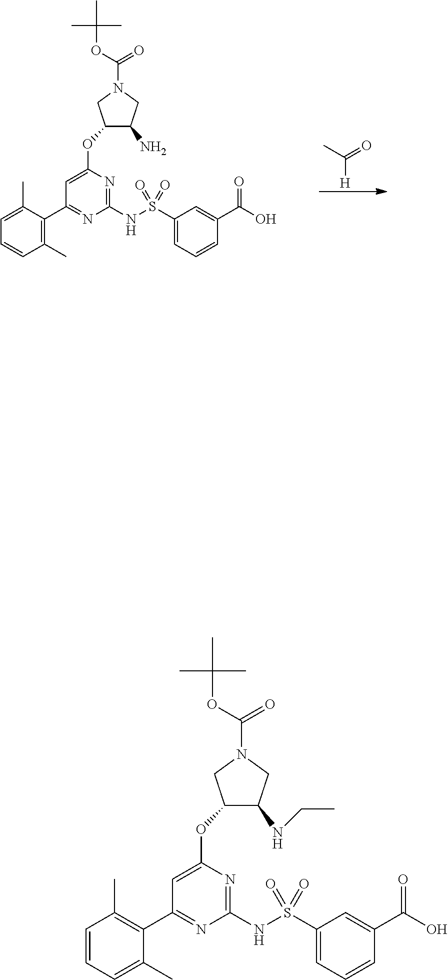

A solution of 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (291.8 mg, 0.5 mmol), acetaldehyde (approximately 24.23 mg, 30.87 μL, 0.5500 mmol), and sodium triacetoxyborohydride (approximately 211.9 mg, 1.000 mmol) in dichloromethane (2.500 mL) was stirred for 18 hours. More acetaldehyde (approximately 24.23 mg, 30.87 μL, 0.5500 mmol) and sodium triacetoxyborohydride (approximately 211.9 mg, 1.000 mmol) were again added, and the reactions were stirred for 22 hours. The solvent was evaporated, and the residue was diluted with water, acidified with 1 M citric acid, and extracted with ethyl acetate. The residue was purified by silica gel column chromatography with 0-8% methanol in dichloromethane to give 3-[[4-[(3R,4R)-1-tert-butoxycarbonyl-4-(ethylamino)pyrrolidin-3-yl]oxy-6-(2,6-dimethylphenyl)pyrimidin-2-yl]sulfamoyl]benzoic acid (44 mg, 14%). ESI-MS m/z calc. 611.2414, found 612.2 (M+1)⁺; Retention time: 0.5 minutes; LC method D.

### Step 2: (3R,7R)-19-(2,6-Dimethylphenyl)-8-ethyl-2-oxa-15λ⁶-thia-5,8,16,18,21-pentaazatetracyclo[15.3.1.110,14.03,7]docosa-1(20),10(22),11,13,17(21),18-hexaene-9,15,15-trione

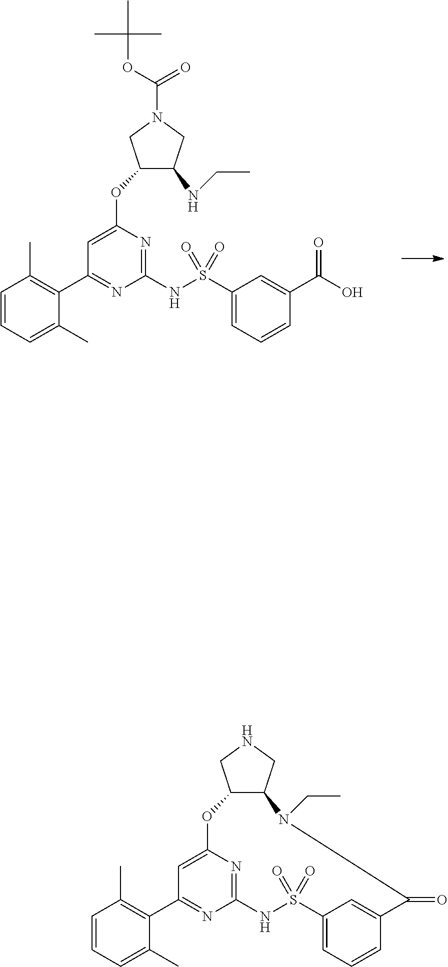 [SMILES: CCN[C@@H]1CN(C(=O)OC(C)(C)C)C[C@H]1Oc1cc(-c2c(C)cccc2C)nc(NS(=O)(=O)c2cccc(C(=O)O)c2)n1 ; CCN1C(=O)c2cccc(c2)S(=O)(=O)Nc2nc(cc(-c3c(C)cccc3C)n2)O[C@@H]2CNC[C@H]21]

A solution of 3-[[4-[(3R,4R)-1-tert-butoxycarbonyl-4-(ethylamino)pyrrolidin-3-yl]oxy-6-(2,6-dimethylphenyl)pyrimidin-2-yl]sulfamoyl]benzoic acid (44 mg, 0.07193 mmol), [[(E)-(1-cyano-2-ethoxy-2-oxo-ethylidene)amino]oxy-tetrahydropyran-4-yl-methylene]-dimethyl-ammonium (Phosphorus Hexafluoride Ion) (49 mg, 0.1147 mmol), and DIEA (38 μL, 0.2182 mmol) in DMF (5 mL) was stirred for three days. The reaction was acidified with 1 M citric acid, diluted with water, and extracted with ethyl acetate. The combined extracts were washed with brine, dried over sodium sulfate, and evaporated under vacuum. The residue was purified by reverse-phase HPLC-MS (1%-99% acetonitrile/water (5 mM HCl)) to give intermediate containing some product. HCl (4 mL of 4 M, 16.00 mmol) (in dioxane) was added, and the reaction was stirred for three hours. The solvent was removed under vacuum to give (3R,7R)-19-(2,6-dimethylphenyl)-8-ethyl-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (13 mg, 34%) as a tan solid. ESI-MS m/z calc. 493.17838, found 494.2 (M+1)⁺; Retention time: 0.3 minutes, LC method D.

### Step 3: (3R,7R)-19-(2,6-Dimethylphenyl)-8-ethyl-5-{spiro[3.4]octan-2-yl}-2-oxa-15)⁶-thia-5,8,16,18,21-pentaazatetracyclo[15.3.1.110,14.03,7]docosa-1(20),10(22),11,13,17(21),18-hexaene-9,15,15-trione (Compound 1192)

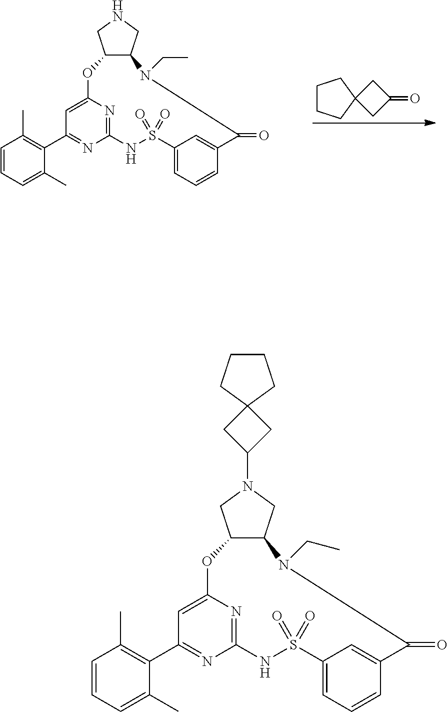 [SMILES: CCN1C(=O)c2cccc(c2)S(=O)(=O)Nc2nc(cc(-c3c(C)cccc3C)n2)O[C@@H]2CNC[C@H]21 ; O=C1CC2(CCCC2)C1 ; CCN1C(=O)c2cccc(c2)S(=O)(=O)Nc2nc(cc(-c3c(C)cccc3C)n2)O[C@@H]2CN(C3CC4(CCCC4)C3)C[C@H]21]

A solution of (3R,7R)-19-(2,6-dimethylphenyl)-8-ethyl-2-oxa-15λ⁶-thia-5,8,16,18,21-pentaazatetracyclo[15.3.1.110,14.03,7]docosa-1(20),10(22),11,13,17(21),18-hexaene-9,15,15-trione (hydrochloride salt) (12 mg, 0.02264 mmol), spiro[3.4]octan-2-one (approximately 8.434 mg, 0.06792 mmol), and sodium triacetoxyborohydride (approximately 19.19 mg, 0.09056 mmol) in dichloromethane (0.3 mL) was stirred for 17 hours. The reaction was stirred with methanol, and the solvent was evaporated. The residue was purified by reverse-phase HPLC-MS (10%-99% acetonitrile/water (5 mM HCl)) to give (3R,7R)-19-(2,6-dimethylphenyl)-8-ethyl ˜5-{spiro[3.4] octan-2-yl}-2-oxa-15λ⁶-thia-5, 8,16,18,21-pentaazatetracyclo[15.3.1.110,14.03,7]docosa-1(20),10(22),11,13,17(21),18-hexaene-9,15,15-trione (hydrochloride salt) (8.4 mg, 61%). ESI-MS m/z calc. 601.2723, found 602.4 (M+1)⁺; Retention time: 1.23 minutes; LC method A.

### Example 135: Characterization of Compounds 158-1193

The compounds in the following tables were prepared in a manner analogous to that described above using commercially available reagents and intermediates described herein.

Lengthy table referenced here
US20240150377A1-20240509-T00001
Please refer to the end of the specification for access instructions.

Lengthy table referenced here
US20240150377A1-20240509-T00002
Please refer to the end of the specification for access instructions.

### Example 136: Compounds 1194 to 1294

Compounds 1194 to 1294, depicted in Table 5, can be prepared following the procedures described above for Compound 1-1193 and CFTR modulating activity can be assessed using one or more of the assays outlined above.

TABLE 5
Compounds 1194-1294

| Compound | |
|---|---|
| Number | Structure |
| 1194 | 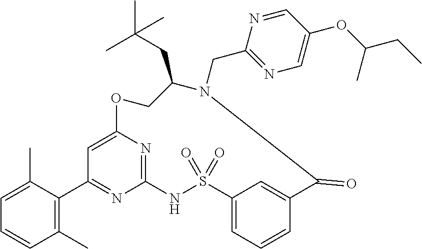 [SMILES: CCC(C)Oc1cnc(CN2C(=O)c3cccc(c3)S(=O)(=O)Nc3nc(cc(-c4c(C)cccc4C)n3)OC[C@H]2CC(C)(C)C)nc1] |
| 1195 | 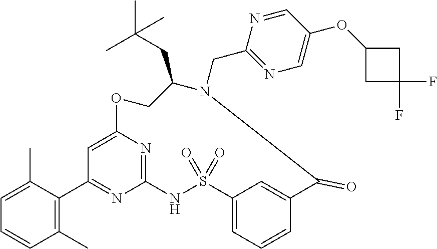 [SMILES: Cc1cccc(C)c1-c1cc2nc(n1)NS(=O)(=O)c1cccc(c1)C(=O)N(Cc1ncc(OC3CC(F)(F)C3)cn1)[C@H](CC(C)(C)C)CO2] |
| 1196 | 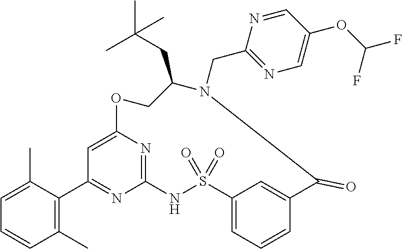 [SMILES: Cc1cccc(C)c1-c1cc2nc(n1)NS(=O)(=O)c1cccc(c1)C(=O)N(Cc1ncc(OC(F)F)cn1)[C@H](CC(C)(C)C)CO2] |
| 1197 | 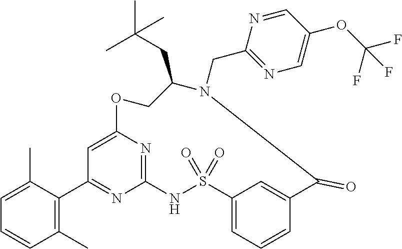 [SMILES: Cc1cccc(C)c1-c1cc2nc(n1)NS(=O)(=O)c1cccc(c1)C(=O)N(Cc1ncc(OC(F)(F)F)cn1)[C@H](CC(C)(C)C)CO2] |
| 1198 | 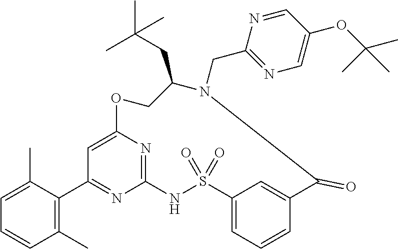 [SMILES: Cc1cccc(C)c1-c1cc2nc(n1)NS(=O)(=O)c1cccc(c1)C(=O)N(Cc1ncc(OC(C)(C)C)cn1)[C@H](CC(C)(C)C)CO2] |
| 1199 | 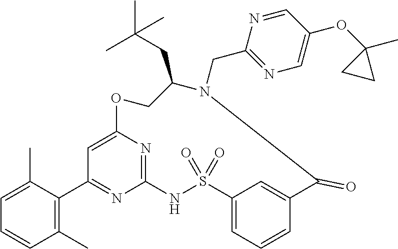 [SMILES: Cc1cccc(C)c1-c1cc2nc(n1)NS(=O)(=O)c1cccc(c1)C(=O)N(Cc1ncc(OC3(C)CC3)cn1)[C@H](CC(C)(C)C)CO2] |
| 1200 | 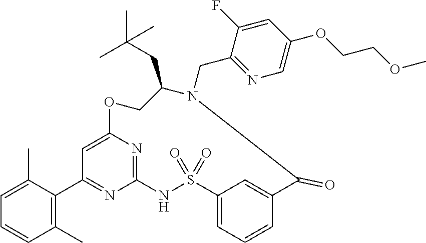 [SMILES: COCCOc1cnc(CN2C(=O)c3cccc(c3)S(=O)(=O)Nc3nc(cc(-c4c(C)cccc4C)n3)OC[C@H]2CC(C)(C)C)c(F)c1] |
| 1201 | 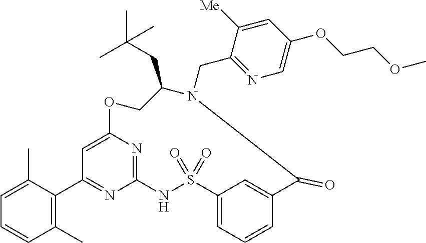 [SMILES: COCCOc1cnc(CN2C(=O)c3cccc(c3)S(=O)(=O)Nc3nc(cc(-c4c(C)cccc4C)n3)OC[C@H]2CC(C)(C)C)c(C)c1] |
| 1202 | 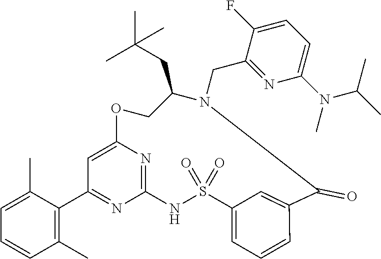 [SMILES: Cc1cccc(C)c1-c1cc2nc(n1)NS(=O)(=O)c1cccc(c1)C(=O)N(Cc1nc(N(C)C(C)C)ccc1F)[C@H](CC(C)(C)C)CO2] |
| 1203 | 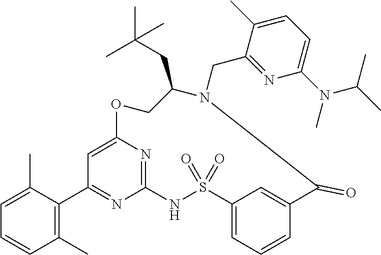 [SMILES: Cc1ccc(N(C)C(C)C)nc1CN1C(=O)c2cccc(c2)S(=O)(=O)Nc2nc(cc(-c3c(C)cccc3C)n2)OC[C@H]1CC(C)(C)C] |
| 1204 | 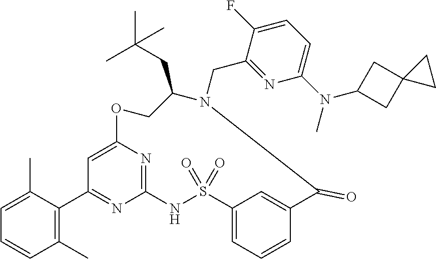 [SMILES: Cc1cccc(C)c1-c1cc2nc(n1)NS(=O)(=O)c1cccc(c1)C(=O)N(Cc1nc(N(C)C3CC4(CC4)C3)ccc1F)[C@H](CC(C)(C)C)CO2] |
| 1205 | 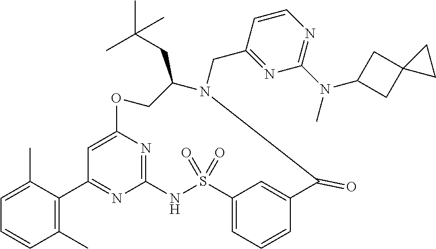 [SMILES: Cc1cccc(C)c1-c1cc2nc(n1)NS(=O)(=O)c1cccc(c1)C(=O)N(Cc1ccnc(N(C)C3CC4(CC4)C3)n1)[C@H](CC(C)(C)C)CO2] |
| 1206 | 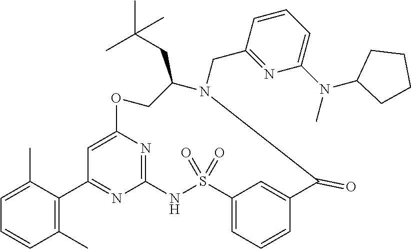 [SMILES: Cc1cccc(C)c1-c1cc2nc(n1)NS(=O)(=O)c1cccc(c1)C(=O)N(Cc1cccc(N(C)C3CCCC3)n1)[C@H](CC(C)(C)C)CO2] |
| 1207 | 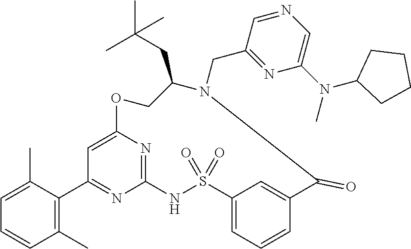 [SMILES: Cc1cccc(C)c1-c1cc2nc(n1)NS(=O)(=O)c1cccc(c1)C(=O)N(Cc1cncc(N(C)C3CCCC3)n1)[C@H](CC(C)(C)C)CO2] |
| 1208 | 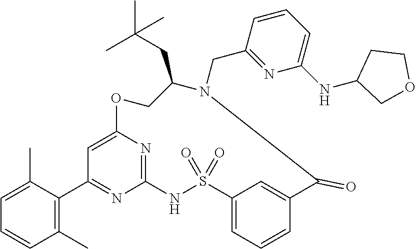 [SMILES: Cc1cccc(C)c1-c1cc2nc(n1)NS(=O)(=O)c1cccc(c1)C(=O)N(Cc1cccc(NC3CCOC3)n1)[C@H](CC(C)(C)C)CO2] |
| 1209 | 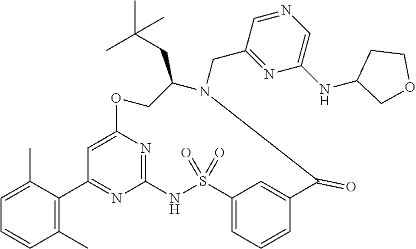 [SMILES: Cc1cccc(C)c1-c1cc2nc(n1)NS(=O)(=O)c1cccc(c1)C(=O)N(Cc1cncc(NC3CCOC3)n1)[C@H](CC(C)(C)C)CO2] |
| 1210 | 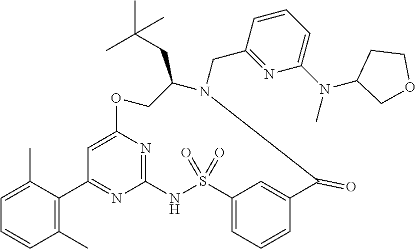 [SMILES: Cc1cccc(C)c1-c1cc2nc(n1)NS(=O)(=O)c1cccc(c1)C(=O)N(Cc1cccc(N(C)C3CCOC3)n1)[C@H](CC(C)(C)C)CO2] |
| 1211 | 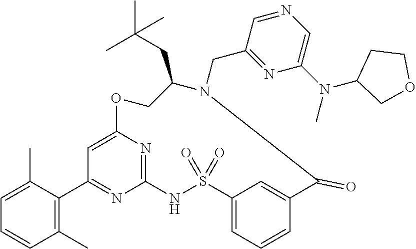 [SMILES: Cc1cccc(C)c1-c1cc2nc(n1)NS(=O)(=O)c1cccc(c1)C(=O)N(Cc1cncc(N(C)C3CCOC3)n1)[C@H](CC(C)(C)C)CO2] |
| 1212 | 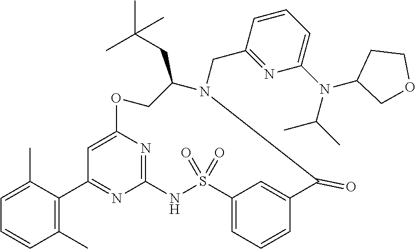 [SMILES: Cc1cccc(C)c1-c1cc2nc(n1)NS(=O)(=O)c1cccc(c1)C(=O)N(Cc1cccc(N(C(C)C)C3CCOC3)n1)[C@H](CC(C)(C)C)CO2] |
| 1213 | 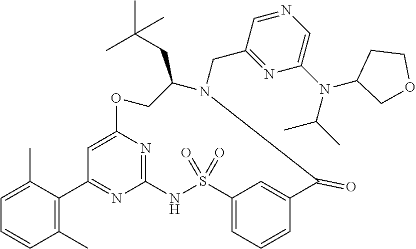 [SMILES: Cc1cccc(C)c1-c1cc2nc(n1)NS(=O)(=O)c1cccc(c1)C(=O)N(Cc1cncc(N(C(C)C)C3CCOC3)n1)[C@H](CC(C)(C)C)CO2] |
| 1214 | 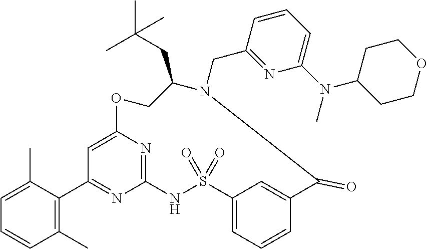 [SMILES: Cc1cccc(C)c1-c1cc2nc(n1)NS(=O)(=O)c1cccc(c1)C(=O)N(Cc1cccc(N(C)C3CCOCC3)n1)[C@H](CC(C)(C)C)CO2] |
| 1215 | 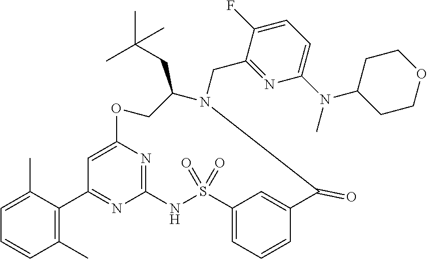 [SMILES: Cc1cccc(C)c1-c1cc2nc(n1)NS(=O)(=O)c1cccc(c1)C(=O)N(Cc1nc(N(C)C3CCOCC3)ccc1F)[C@H](CC(C)(C)C)CO2] |
| 1216 | 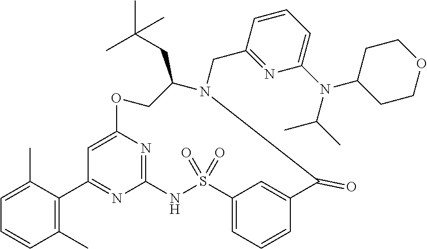 [SMILES: Cc1cccc(C)c1-c1cc2nc(n1)NS(=O)(=O)c1cccc(c1)C(=O)N(Cc1cccc(N(C(C)C)C3CCOCC3)n1)[C@H](CC(C)(C)C)CO2] |
| 1217 | 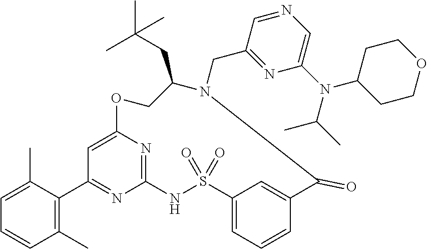 [SMILES: Cc1cccc(C)c1-c1cc2nc(n1)NS(=O)(=O)c1cccc(c1)C(=O)N(Cc1cncc(N(C(C)C)C3CCOCC3)n1)[C@H](CC(C)(C)C)CO2] |
| 1218 | 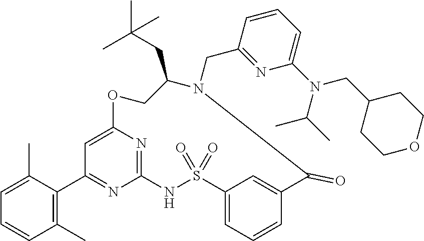 [SMILES: Cc1cccc(C)c1-c1cc2nc(n1)NS(=O)(=O)c1cccc(c1)C(=O)N(Cc1cccc(N(CC3CCOCC3)C(C)C)n1)[C@H](CC(C)(C)C)CO2] |
| 1219 | 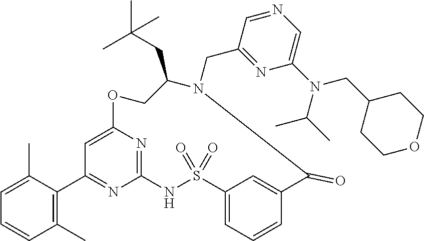 [SMILES: Cc1cccc(C)c1-c1cc2nc(n1)NS(=O)(=O)c1cccc(c1)C(=O)N(Cc1cncc(N(CC3CCOCC3)C(C)C)n1)[C@H](CC(C)(C)C)CO2] |
| 1220 | 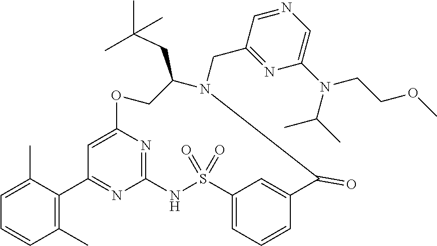 [SMILES: COCCN(c1cncc(CN2C(=O)c3cccc(c3)S(=O)(=O)Nc3nc(cc(-c4c(C)cccc4C)n3)OC[C@H]2CC(C)(C)C)n1)C(C)C] |
| 1221 | 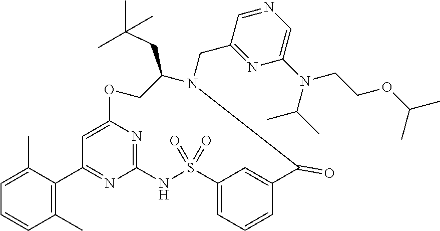 [SMILES: Cc1cccc(C)c1-c1cc2nc(n1)NS(=O)(=O)c1cccc(c1)C(=O)N(Cc1cncc(N(CCOC(C)C)C(C)C)n1)[C@H](CC(C)(C)C)CO2] |
| 1222 | 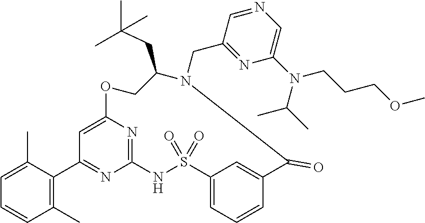 [SMILES: COCCCN(c1cncc(CN2C(=O)c3cccc(c3)S(=O)(=O)Nc3nc(cc(-c4c(C)cccc4C)n3)OC[C@H]2CC(C)(C)C)n1)C(C)C] |
| 1223 | 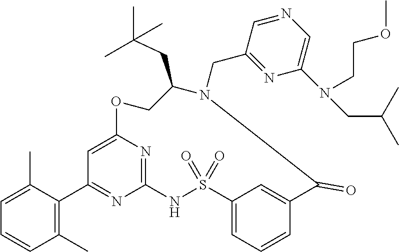 [SMILES: COCCN(CC(C)C)c1cncc(CN2C(=O)c3cccc(c3)S(=O)(=O)Nc3nc(cc(-c4c(C)cccc4C)n3)OC[C@H]2CC(C)(C)C)n1] |
| 1224 | 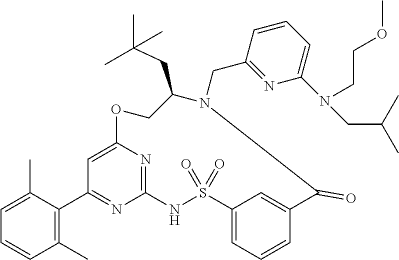 [SMILES: COCCN(CC(C)C)c1cccc(CN2C(=O)c3cccc(c3)S(=O)(=O)Nc3nc(cc(-c4c(C)cccc4C)n3)OC[C@H]2CC(C)(C)C)n1] |
| 1225 | 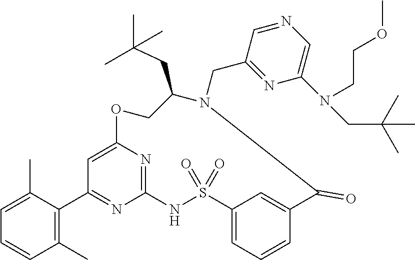 [SMILES: COCCN(CC(C)(C)C)c1cncc(CN2C(=O)c3cccc(c3)S(=O)(=O)Nc3nc(cc(-c4c(C)cccc4C)n3)OC[C@H]2CC(C)(C)C)n1] |
| 1226 | 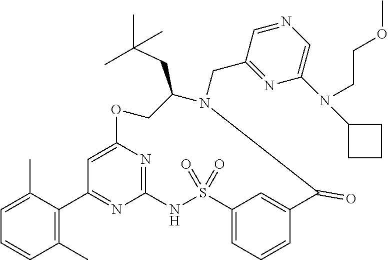 [SMILES: COCCN(c1cncc(CN2C(=O)c3cccc(c3)S(=O)(=O)Nc3nc(cc(-c4c(C)cccc4C)n3)OC[C@H]2CC(C)(C)C)n1)C1CCC1] |
| 1227 | 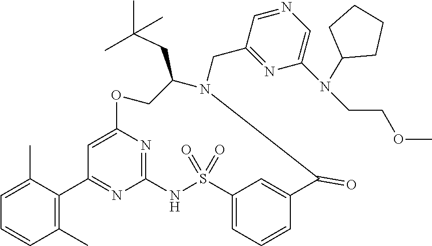 [SMILES: COCCN(c1cncc(CN2C(=O)c3cccc(c3)S(=O)(=O)Nc3nc(cc(-c4c(C)cccc4C)n3)OC[C@H]2CC(C)(C)C)n1)C1CCCC1] |
| 1228 | 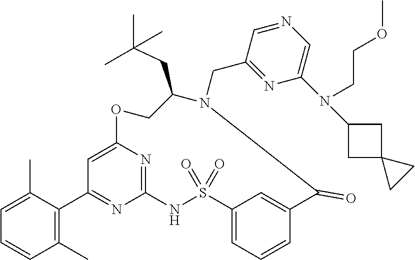 [SMILES: COCCN(c1cncc(CN2C(=O)c3cccc(c3)S(=O)(=O)Nc3nc(cc(-c4c(C)cccc4C)n3)OC[C@H]2CC(C)(C)C)n1)C1CC2(CC2)C1] |
| 1229 | 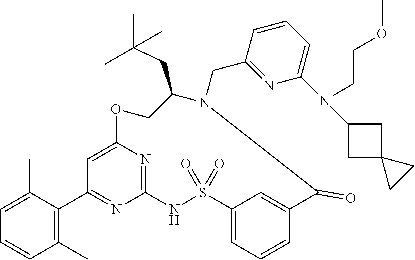 [SMILES: COCCN(c1cccc(CN2C(=O)c3cccc(c3)S(=O)(=O)Nc3nc(cc(-c4c(C)cccc4C)n3)OC[C@H]2CC(C)(C)C)n1)C1CC2(CC2)C1] |
| 1230 | 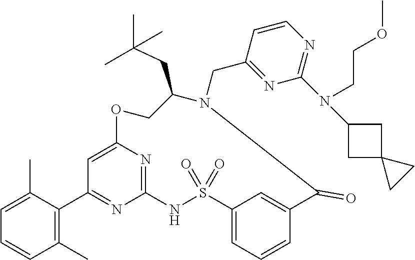 [SMILES: COCCN(c1nccc(CN2C(=O)c3cccc(c3)S(=O)(=O)Nc3nc(cc(-c4c(C)cccc4C)n3)OC[C@H]2CC(C)(C)C)n1)C1CC2(CC2)C1] |
| 1231 | 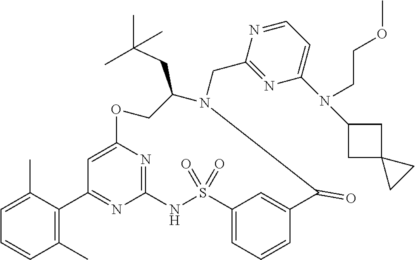 [SMILES: COCCN(c1ccnc(CN2C(=O)c3cccc(c3)S(=O)(=O)Nc3nc(cc(-c4c(C)cccc4C)n3)OC[C@H]2CC(C)(C)C)n1)C1CC2(CC2)C1] |
| 1232 | 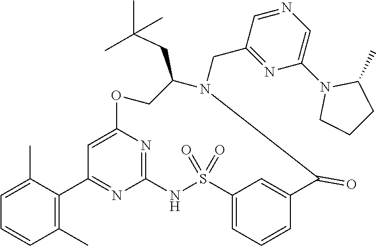 [SMILES: Cc1cccc(C)c1-c1cc2nc(n1)NS(=O)(=O)c1cccc(c1)C(=O)N(Cc1cncc(N3CCC[C@H]3C)n1)[C@H](CC(C)(C)C)CO2] |
| 1233 | 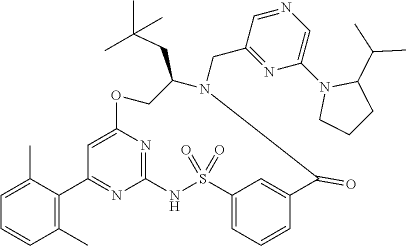 [SMILES: Cc1cccc(C)c1-c1cc2nc(n1)NS(=O)(=O)c1cccc(c1)C(=O)N(Cc1cncc(N3CCCC3C(C)C)n1)[C@H](CC(C)(C)C)CO2] |
| 1234 | 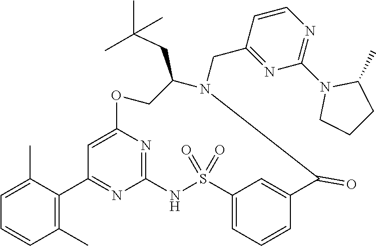 [SMILES: Cc1cccc(C)c1-c1cc2nc(n1)NS(=O)(=O)c1cccc(c1)C(=O)N(Cc1ccnc(N3CCC[C@H]3C)n1)[C@H](CC(C)(C)C)CO2] |
| 1235 | 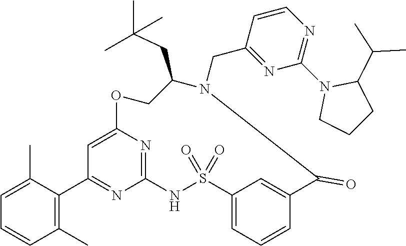 [SMILES: Cc1cccc(C)c1-c1cc2nc(n1)NS(=O)(=O)c1cccc(c1)C(=O)N(Cc1ccnc(N3CCCC3C(C)C)n1)[C@H](CC(C)(C)C)CO2] |
| 1236 | 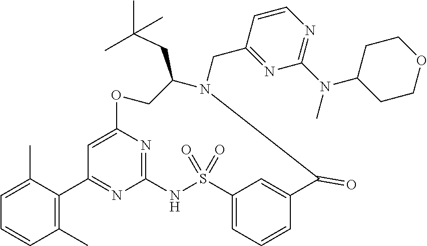 [SMILES: Cc1cccc(C)c1-c1cc2nc(n1)NS(=O)(=O)c1cccc(c1)C(=O)N(Cc1ccnc(N(C)C3CCOCC3)n1)[C@H](CC(C)(C)C)CO2] |
| 1237 | 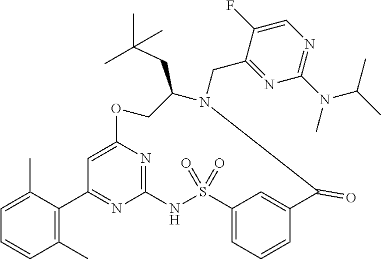 [SMILES: Cc1cccc(C)c1-c1cc2nc(n1)NS(=O)(=O)c1cccc(c1)C(=O)N(Cc1nc(N(C)C(C)C)ncc1F)[C@H](CC(C)(C)C)CO2] |
| 1238 | 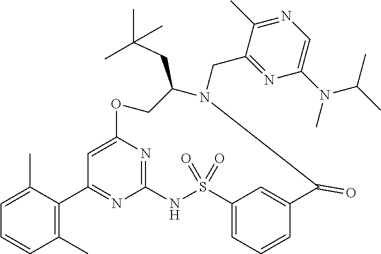 [SMILES: Cc1cccc(C)c1-c1cc2nc(n1)NS(=O)(=O)c1cccc(c1)C(=O)N(Cc1nc(N(C)C(C)C)cnc1C)[C@H](CC(C)(C)C)CO2] |
| 1239 | 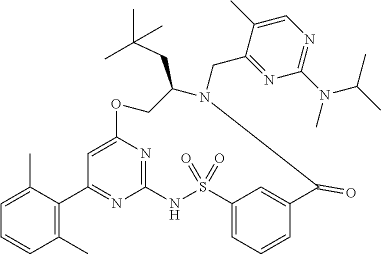 [SMILES: Cc1cnc(N(C)C(C)C)nc1CN1C(=O)c2cccc(c2)S(=O)(=O)Nc2nc(cc(-c3c(C)cccc3C)n2)OC[C@H]1CC(C)(C)C] |
| 1240 | 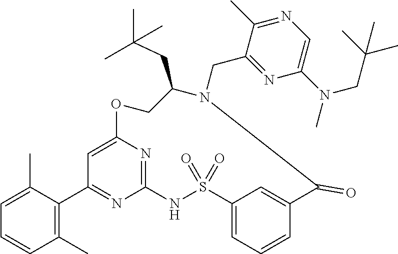 [SMILES: Cc1cccc(C)c1-c1cc2nc(n1)NS(=O)(=O)c1cccc(c1)C(=O)N(Cc1nc(N(C)CC(C)(C)C)cnc1C)[C@H](CC(C)(C)C)CO2] |
| 1241 | 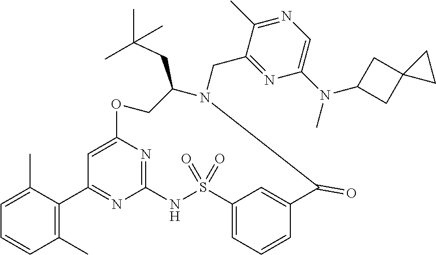 [SMILES: Cc1cccc(C)c1-c1cc2nc(n1)NS(=O)(=O)c1cccc(c1)C(=O)N(Cc1nc(N(C)C3CC4(CC4)C3)cnc1C)[C@H](CC(C)(C)C)CO2] |
| 1242 | 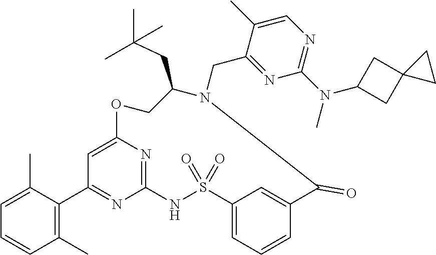 [SMILES: Cc1cnc(N(C)C2CC3(CC3)C2)nc1CN1C(=O)c2cccc(c2)S(=O)(=O)Nc2nc(cc(-c3c(C)cccc3C)n2)OC[C@H]1CC(C)(C)C] |
| 1243 | 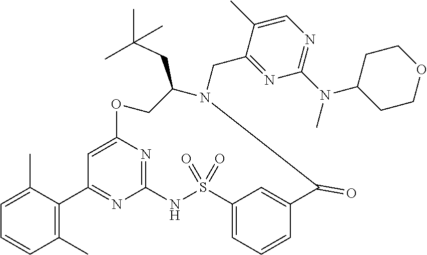 [SMILES: Cc1cnc(N(C)C2CCOCC2)nc1CN1C(=O)c2cccc(c2)S(=O)(=O)Nc2nc(cc(-c3c(C)cccc3C)n2)OC[C@H]1CC(C)(C)C] |
| 1244 | 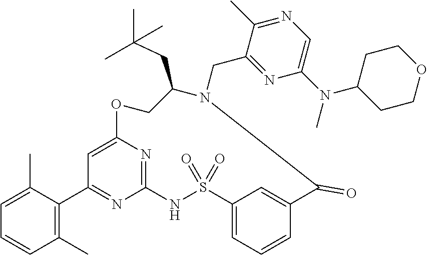 [SMILES: Cc1cccc(C)c1-c1cc2nc(n1)NS(=O)(=O)c1cccc(c1)C(=O)N(Cc1nc(N(C)C3CCOCC3)cnc1C)[C@H](CC(C)(C)C)CO2] |
| 1245 | 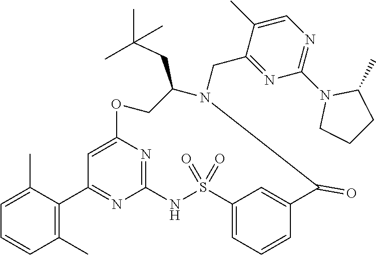 [SMILES: Cc1cnc(N2CCC[C@H]2C)nc1CN1C(=O)c2cccc(c2)S(=O)(=O)Nc2nc(cc(-c3c(C)cccc3C)n2)OC[C@H]1CC(C)(C)C] |
| 1246 | 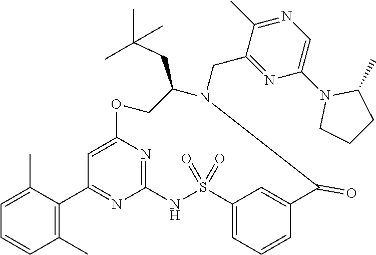 [SMILES: Cc1cccc(C)c1-c1cc2nc(n1)NS(=O)(=O)c1cccc(c1)C(=O)N(Cc1nc(N3CCC[C@H]3C)cnc1C)[C@H](CC(C)(C)C)CO2] |
| 1247 | 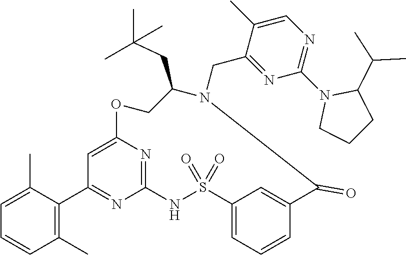 [SMILES: Cc1cnc(N2CCCC2C(C)C)nc1CN1C(=O)c2cccc(c2)S(=O)(=O)Nc2nc(cc(-c3c(C)cccc3C)n2)OC[C@H]1CC(C)(C)C] |
| 1248 | 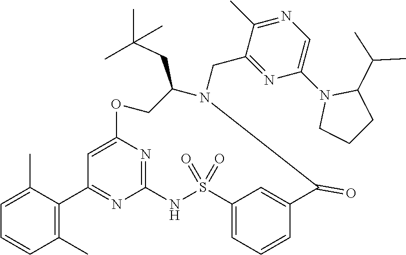 [SMILES: Cc1cccc(C)c1-c1cc2nc(n1)NS(=O)(=O)c1cccc(c1)C(=O)N(Cc1nc(N3CCCC3C(C)C)cnc1C)[C@H](CC(C)(C)C)CO2] |
| 1249 | 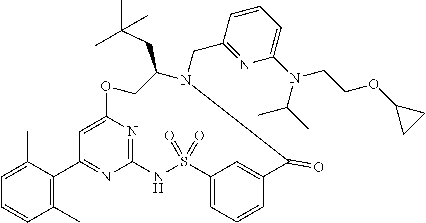 [SMILES: Cc1cccc(C)c1-c1cc2nc(n1)NS(=O)(=O)c1cccc(c1)C(=O)N(Cc1cccc(N(CCOC3CC3)C(C)C)n1)[C@H](CC(C)(C)C)CO2] |
| 1250 | 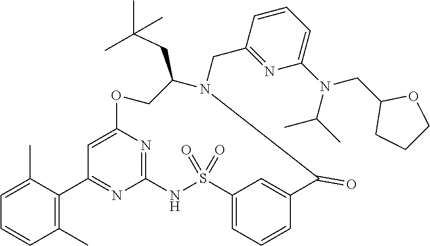 [SMILES: Cc1cccc(C)c1-c1cc2nc(n1)NS(=O)(=O)c1cccc(c1)C(=O)N(Cc1cccc(N(CC3CCCO3)C(C)C)n1)[C@H](CC(C)(C)C)CO2] |
| 1251 | 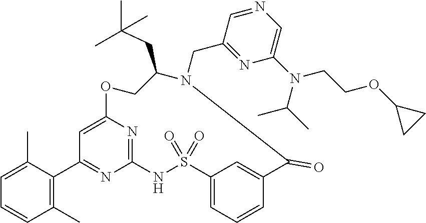 [SMILES: Cc1cccc(C)c1-c1cc2nc(n1)NS(=O)(=O)c1cccc(c1)C(=O)N(Cc1cncc(N(CCOC3CC3)C(C)C)n1)[C@H](CC(C)(C)C)CO2] |
| 1252 | 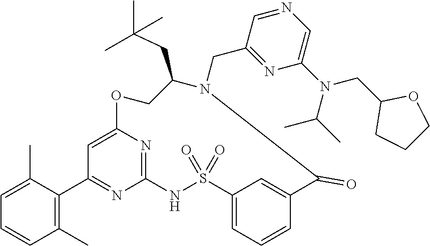 [SMILES: Cc1cccc(C)c1-c1cc2nc(n1)NS(=O)(=O)c1cccc(c1)C(=O)N(Cc1cncc(N(CC3CCCO3)C(C)C)n1)[C@H](CC(C)(C)C)CO2] |
| 1253 | 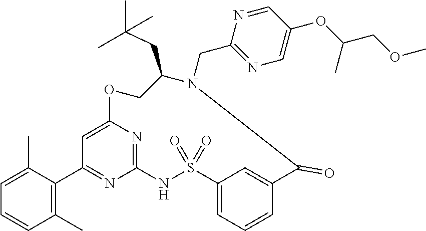 [SMILES: COCC(C)Oc1cnc(CN2C(=O)c3cccc(c3)S(=O)(=O)Nc3nc(cc(-c4c(C)cccc4C)n3)OC[C@H]2CC(C)(C)C)nc1] |
| 1254 | 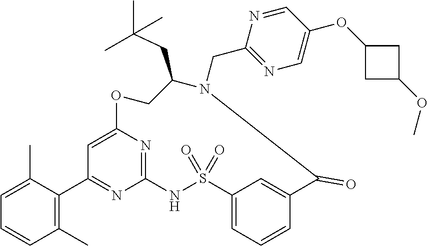 [SMILES: COC1CC(Oc2cnc(CN3C(=O)c4cccc(c4)S(=O)(=O)Nc4nc(cc(-c5c(C)cccc5C)n4)OC[C@H]3CC(C)(C)C)nc2)C1] |
| 1255 | 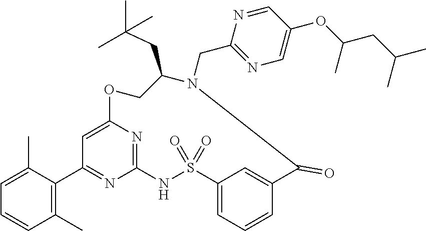 [SMILES: Cc1cccc(C)c1-c1cc2nc(n1)NS(=O)(=O)c1cccc(c1)C(=O)N(Cc1ncc(OC(C)CC(C)C)cn1)[C@H](CC(C)(C)C)CO2] |
| 1256 | 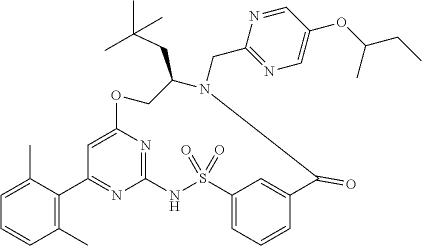 [SMILES: CCC(C)Oc1cnc(CN2C(=O)c3cccc(c3)S(=O)(=O)Nc3nc(cc(-c4c(C)cccc4C)n3)OC[C@H]2CC(C)(C)C)nc1] |
| 1257 | 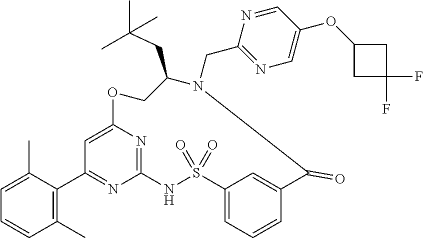 [SMILES: Cc1cccc(C)c1-c1cc2nc(n1)NS(=O)(=O)c1cccc(c1)C(=O)N(Cc1ncc(OC3CC(F)(F)C3)cn1)[C@H](CC(C)(C)C)CO2] |
| 1258 | 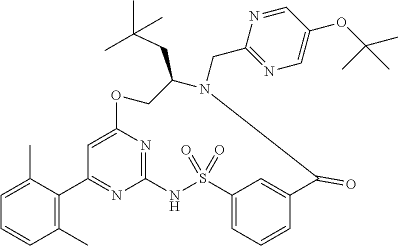 [SMILES: Cc1cccc(C)c1-c1cc2nc(n1)NS(=O)(=O)c1cccc(c1)C(=O)N(Cc1ncc(OC(C)(C)C)cn1)[C@H](CC(C)(C)C)CO2] |
| 1259 | 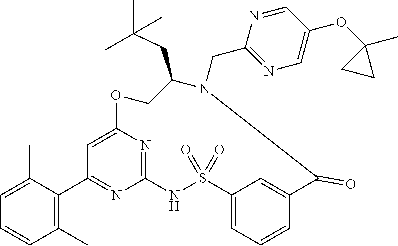 [SMILES: Cc1cccc(C)c1-c1cc2nc(n1)NS(=O)(=O)c1cccc(c1)C(=O)N(Cc1ncc(OC3(C)CC3)cn1)[C@H](CC(C)(C)C)CO2] |
| 1260 | 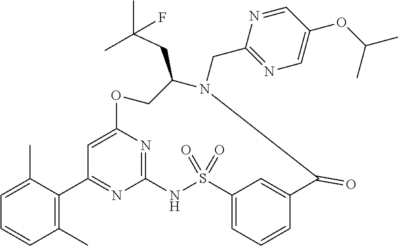 [SMILES: Cc1cccc(C)c1-c1cc2nc(n1)NS(=O)(=O)c1cccc(c1)C(=O)N(Cc1ncc(OC(C)C)cn1)[C@H](CC(C)(C)F)CO2] |
| 1261 | 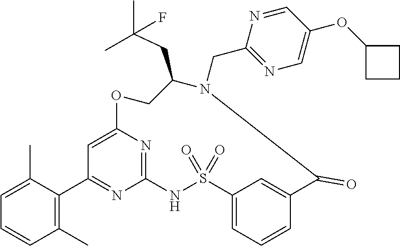 [SMILES: Cc1cccc(C)c1-c1cc2nc(n1)NS(=O)(=O)c1cccc(c1)C(=O)N(Cc1ncc(OC3CCC3)cn1)[C@H](CC(C)(C)F)CO2] |
| 1262 | 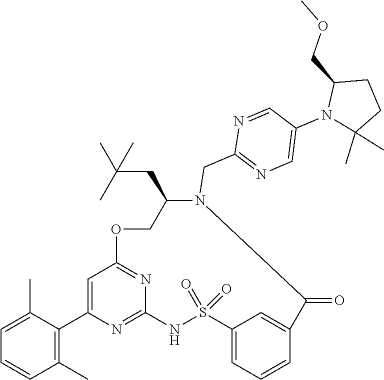 [SMILES: COC[C@H]1CCC(C)(C)N1c1cnc(CN2C(=O)c3cccc(c3)S(=O)(=O)Nc3nc(cc(-c4c(C)cccc4C)n3)OC[C@H]2CC(C)(C)C)nc1] |
| 1263 | 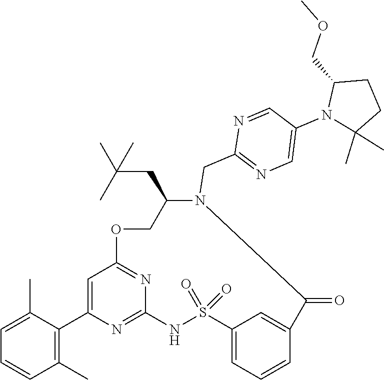 [SMILES: COC[C@@H]1CCC(C)(C)N1c1cnc(CN2C(=O)c3cccc(c3)S(=O)(=O)Nc3nc(cc(-c4c(C)cccc4C)n3)OC[C@H]2CC(C)(C)C)nc1] |
| 1264 | 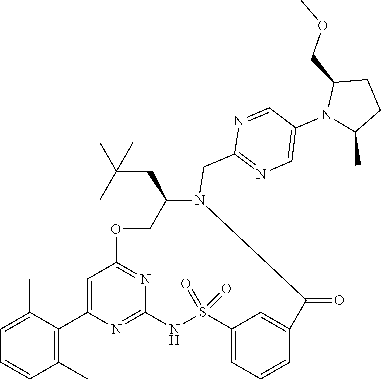 [SMILES: COC[C@H]1CC[C@@H](C)N1c1cnc(CN2C(=O)c3cccc(c3)S(=O)(=O)Nc3nc(cc(-c4c(C)cccc4C)n3)OC[C@H]2CC(C)(C)C)nc1] |
| 1265 | 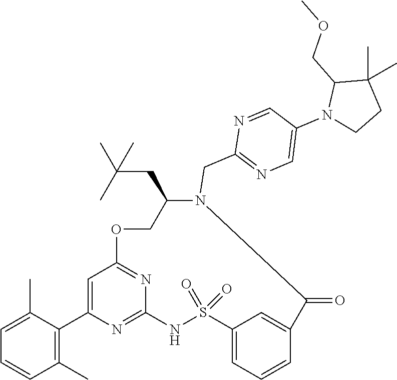 [SMILES: COCC1N(c2cnc(CN3C(=O)c4cccc(c4)S(=O)(=O)Nc4nc(cc(-c5c(C)cccc5C)n4)OC[C@H]3CC(C)(C)C)nc2)CCC1(C)C] |
| 1266 | 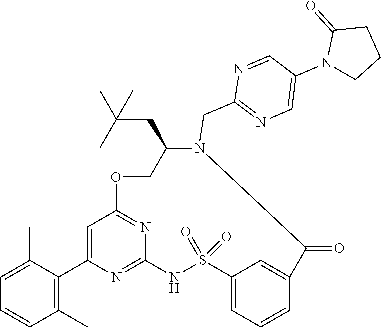 [SMILES: Cc1cccc(C)c1-c1cc2nc(n1)NS(=O)(=O)c1cccc(c1)C(=O)N(Cc1ncc(N3CCCC3=O)cn1)[C@H](CC(C)(C)C)CO2] |
| 1267 | 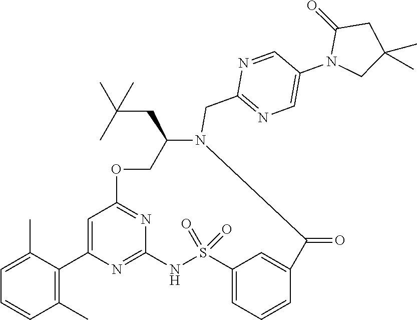 [SMILES: Cc1cccc(C)c1-c1cc2nc(n1)NS(=O)(=O)c1cccc(c1)C(=O)N(Cc1ncc(N3CC(C)(C)CC3=O)cn1)[C@H](CC(C)(C)C)CO2] |
| 1268 | 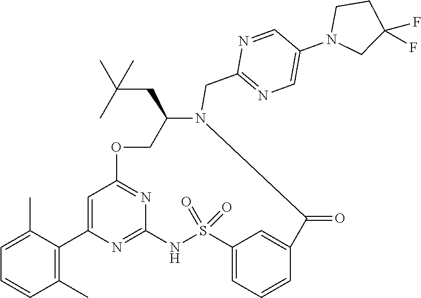 [SMILES: Cc1cccc(C)c1-c1cc2nc(n1)NS(=O)(=O)c1cccc(c1)C(=O)N(Cc1ncc(N3CCC(F)(F)C3)cn1)[C@H](CC(C)(C)C)CO2] |
| 1269 | 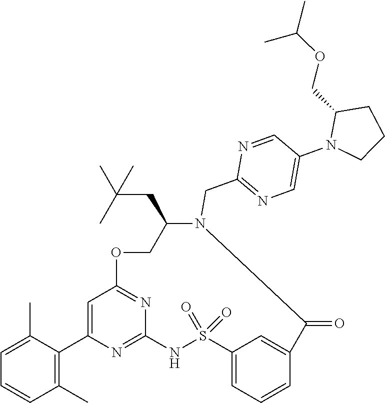 [SMILES: Cc1cccc(C)c1-c1cc2nc(n1)NS(=O)(=O)c1cccc(c1)C(=O)N(Cc1ncc(N3CCC[C@H]3COC(C)C)cn1)[C@H](CC(C)(C)C)CO2] |
| 1270 | 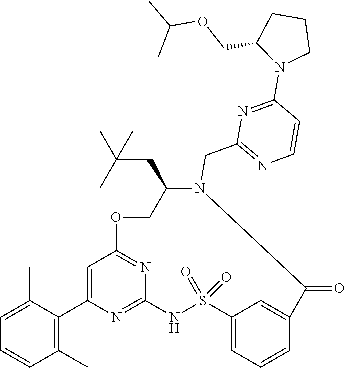 [SMILES: Cc1cccc(C)c1-c1cc2nc(n1)NS(=O)(=O)c1cccc(c1)C(=O)N(Cc1nccc(N3CCC[C@H]3COC(C)C)n1)[C@H](CC(C)(C)C)CO2] |
| 1271 | 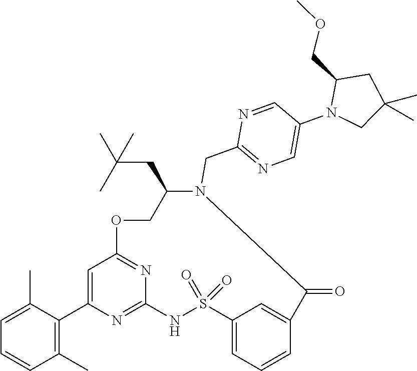 [SMILES: COC[C@H]1CC(C)(C)CN1c1cnc(CN2C(=O)c3cccc(c3)S(=O)(=O)Nc3nc(cc(-c4c(C)cccc4C)n3)OC[C@H]2CC(C)(C)C)nc1] |
| 1272 | 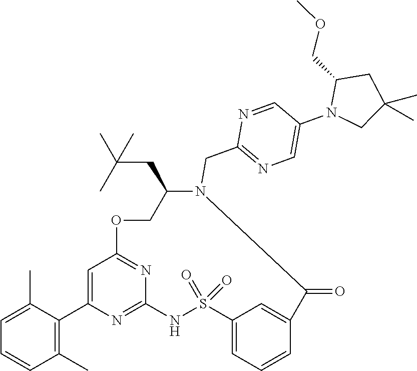 [SMILES: COC[C@@H]1CC(C)(C)CN1c1cnc(CN2C(=O)c3cccc(c3)S(=O)(=O)Nc3nc(cc(-c4c(C)cccc4C)n3)OC[C@H]2CC(C)(C)C)nc1] |
| 1273 | 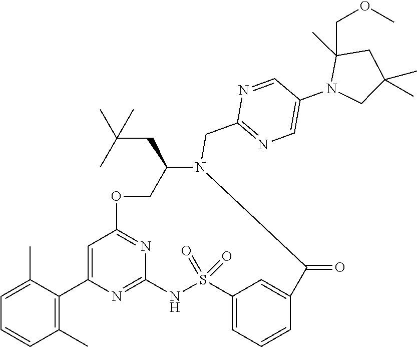 [SMILES: COCC1(C)CC(C)(C)CN1c1cnc(CN2C(=O)c3cccc(c3)S(=O)(=O)Nc3nc(cc(-c4c(C)cccc4C)n3)OC[C@H]2CC(C)(C)C)nc1] |
| 1274 | 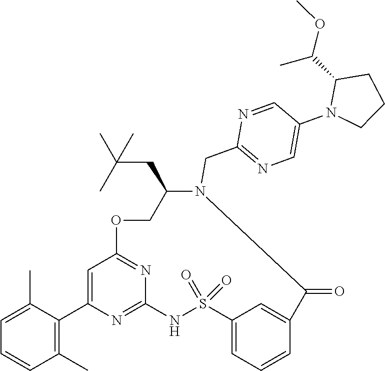 [SMILES: COC(C)[C@@H]1CCCN1c1cnc(CN2C(=O)c3cccc(c3)S(=O)(=O)Nc3nc(cc(-c4c(C)cccc4C)n3)OC[C@H]2CC(C)(C)C)nc1] |
| 1275 | 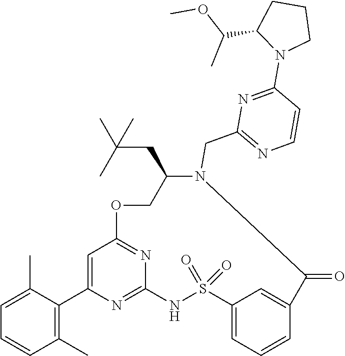 [SMILES: COC(C)[C@@H]1CCCN1c1ccnc(CN2C(=O)c3cccc(c3)S(=O)(=O)Nc3nc(cc(-c4c(C)cccc4C)n3)OC[C@H]2CC(C)(C)C)n1] |
| 1276 | 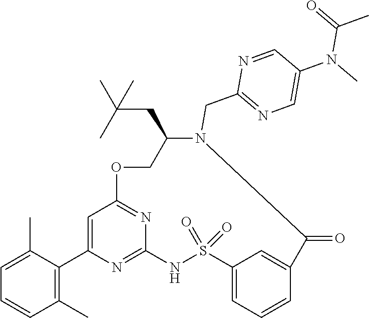 [SMILES: CC(=O)N(C)c1cnc(CN2C(=O)c3cccc(c3)S(=O)(=O)Nc3nc(cc(-c4c(C)cccc4C)n3)OC[C@H]2CC(C)(C)C)nc1] |
| 1277 | 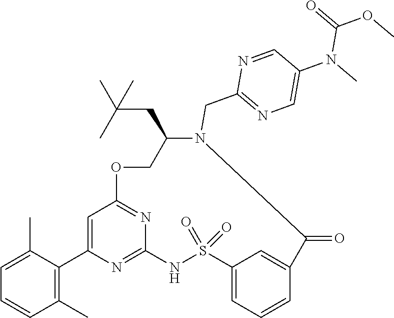 [SMILES: COC(=O)N(C)c1cnc(CN2C(=O)c3cccc(c3)S(=O)(=O)Nc3nc(cc(-c4c(C)cccc4C)n3)OC[C@H]2CC(C)(C)C)nc1] |
| 1278 | 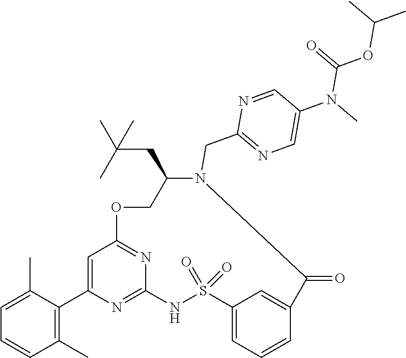 [SMILES: Cc1cccc(C)c1-c1cc2nc(n1)NS(=O)(=O)c1cccc(c1)C(=O)N(Cc1ncc(N(C)C(=O)OC(C)C)cn1)[C@H](CC(C)(C)C)CO2] |
| 1279 | 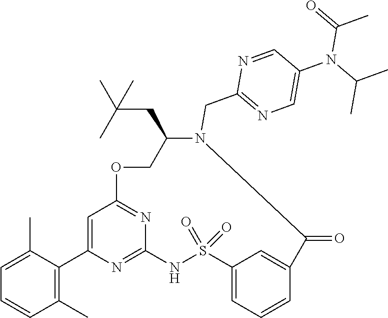 [SMILES: CC(=O)N(c1cnc(CN2C(=O)c3cccc(c3)S(=O)(=O)Nc3nc(cc(-c4c(C)cccc4C)n3)OC[C@H]2CC(C)(C)C)nc1)C(C)C] |
| 1280 | 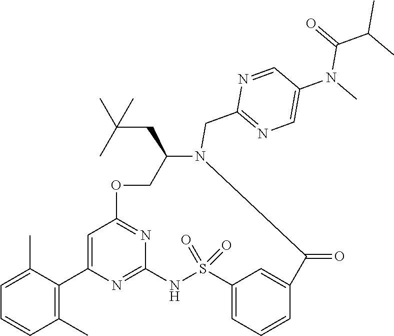 [SMILES: Cc1cccc(C)c1-c1cc2nc(n1)NS(=O)(=O)c1cccc(c1)C(=O)N(Cc1ncc(N(C)C(=O)C(C)C)cn1)[C@H](CC(C)(C)C)CO2] |
| 1281 | 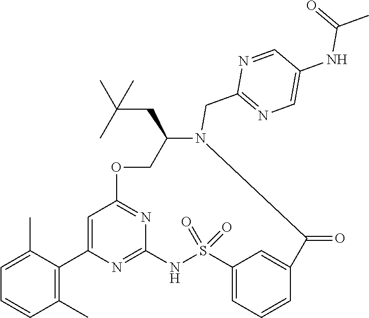 [SMILES: CC(=O)Nc1cnc(CN2C(=O)c3cccc(c3)S(=O)(=O)Nc3nc(cc(-c4c(C)cccc4C)n3)OC[C@H]2CC(C)(C)C)nc1] |
| 1282 | 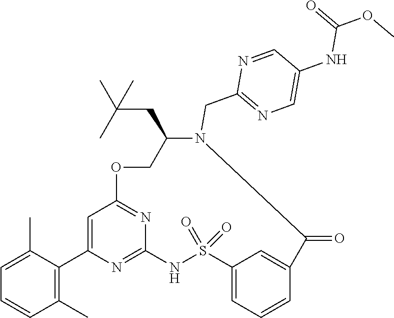 [SMILES: COC(=O)Nc1cnc(CN2C(=O)c3cccc(c3)S(=O)(=O)Nc3nc(cc(-c4c(C)cccc4C)n3)OC[C@H]2CC(C)(C)C)nc1] |
| 1283 | 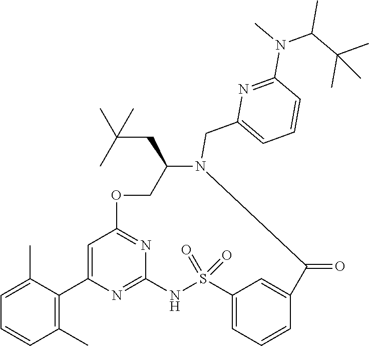 [SMILES: Cc1cccc(C)c1-c1cc2nc(n1)NS(=O)(=O)c1cccc(c1)C(=O)N(Cc1cccc(N(C)C(C)C(C)(C)C)n1)[C@H](CC(C)(C)C)CO2] |
| 1284 | 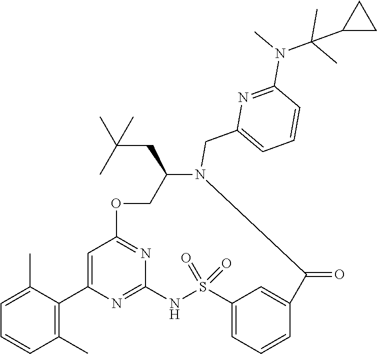 [SMILES: Cc1cccc(C)c1-c1cc2nc(n1)NS(=O)(=O)c1cccc(c1)C(=O)N(Cc1cccc(N(C)C(C)(C)C3CC3)n1)[C@H](CC(C)(C)C)CO2] |
| 1285 | 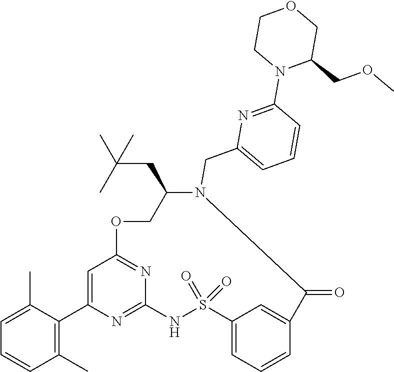 [SMILES: COC[C@@H]1COCCN1c1cccc(CN2C(=O)c3cccc(c3)S(=O)(=O)Nc3nc(cc(-c4c(C)cccc4C)n3)OC[C@H]2CC(C)(C)C)n1] |
| 1286 | 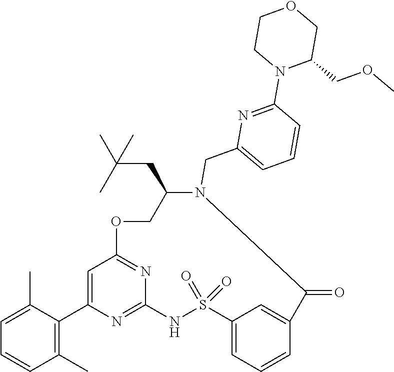 [SMILES: COC[C@H]1COCCN1c1cccc(CN2C(=O)c3cccc(c3)S(=O)(=O)Nc3nc(cc(-c4c(C)cccc4C)n3)OC[C@H]2CC(C)(C)C)n1] |
| 1287 | 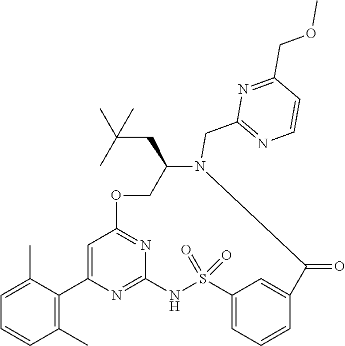 [SMILES: COCc1ccnc(CN2C(=O)c3cccc(c3)S(=O)(=O)Nc3nc(cc(-c4c(C)cccc4C)n3)OC[C@H]2CC(C)(C)C)n1] |
| 1288 | 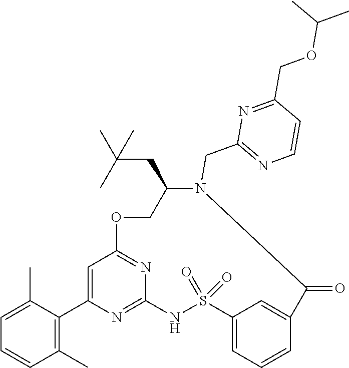 [SMILES: Cc1cccc(C)c1-c1cc2nc(n1)NS(=O)(=O)c1cccc(c1)C(=O)N(Cc1nccc(COC(C)C)n1)[C@H](CC(C)(C)C)CO2] |
| 1289 | 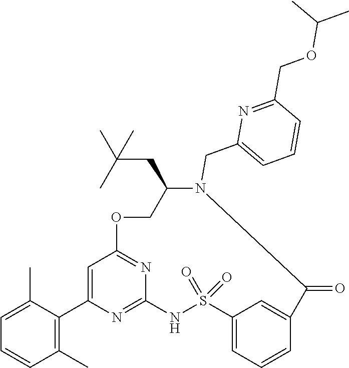 [SMILES: Cc1cccc(C)c1-c1cc2nc(n1)NS(=O)(=O)c1cccc(c1)C(=O)N(Cc1cccc(COC(C)C)n1)[C@H](CC(C)(C)C)CO2] |
| 1290 | 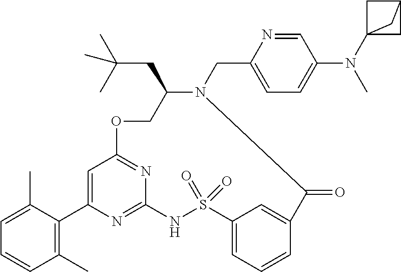 [SMILES: Cc1cccc(C)c1-c1cc2nc(n1)NS(=O)(=O)c1cccc(c1)C(=O)N(Cc1ccc(N(C)C34CC(C3)C4)cn1)[C@H](CC(C)(C)C)CO2] |
| 1291 | 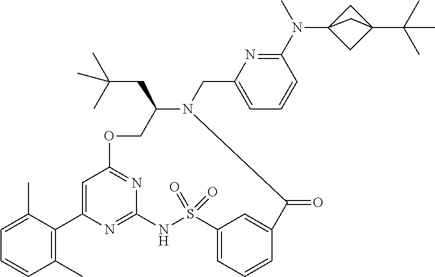 [SMILES: Cc1cccc(C)c1-c1cc2nc(n1)NS(=O)(=O)c1cccc(c1)C(=O)N(Cc1cccc(N(C)C34CC(C(C)(C)C)(C3)C4)n1)[C@H](CC(C)(C)C)CO2] |
| 1292 | 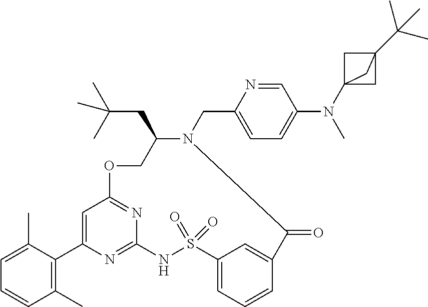 [SMILES: Cc1cccc(C)c1-c1cc2nc(n1)NS(=O)(=O)c1cccc(c1)C(=O)N(Cc1ccc(N(C)C34CC(C(C)(C)C)(C3)C4)cn1)[C@H](CC(C)(C)C)CO2] |
| 1293 | 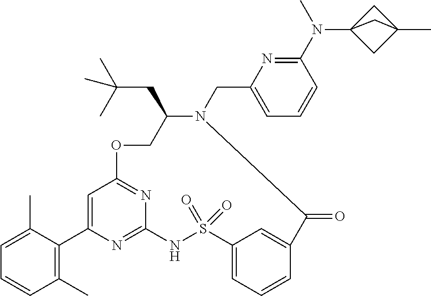 [SMILES: Cc1cccc(C)c1-c1cc2nc(n1)NS(=O)(=O)c1cccc(c1)C(=O)N(Cc1cccc(N(C)C34CC(C)(C3)C4)n1)[C@H](CC(C)(C)C)CO2] |
| 1294 | 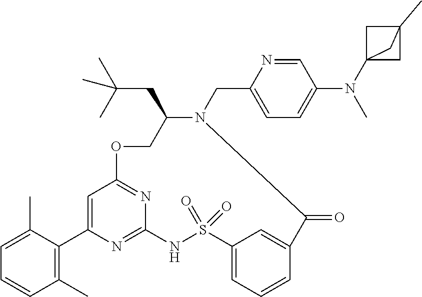 [SMILES: Cc1cccc(C)c1-c1cc2nc(n1)NS(=O)(=O)c1cccc(c1)C(=O)N(Cc1ccc(N(C)C34CC(C)(C3)C4)cn1)[C@H](CC(C)(C)C)CO2] |

### VI. Bioactivity Data for Compounds 1-1193

A. 3t3 Assay

1. Membrane Potential Optical Methods for Assaying F508del Modulation Properties of Compounds

The assay utilizes fluorescent voltage sensing dyes to measure changes in membrane potential using a fluorescent plate reader (e.g., FLIPR III, Molecular Devices, Inc.) as a readout for increase in functional F508del in NIH 3T3 cells. The driving force for the response is the creation of a chloride ion gradient in conjunction with channel activation by a single liquid addition step after the cells have previously been treated with compounds and subsequently loaded with a voltage sensing dye.

2. Identification of Corrector Compounds

To identify correctors of F508del, a single-addition HTS assay format was developed. This HTS assay utilizes fluorescent voltage sensing dyes to measure changes in membrane potential on the FLIPR III as a measurement for increase in gating (conductance) of F508del in F508del NIH 3T3 cells. The F508del NIH 3T3 cell cultures were incubated with the corrector compounds at a range of concentrations for 18-24 hours at 37° C., and subsequently loaded with a redistribution dye. The driving force for the response is a Cl⁻ ion gradient in conjunction with channel activation with forskolin in a single liquid addition step using a fluorescent plate reader such as FLIPR III. The efficacy and potency of the putative F508del correctors was compared to that of the known corrector, lumacaftor, in combination with acutely added 300 nM Ivacaftor.

3. Solutions

Bath Solution #1: (in mM) NaCl 160, KCl 4.5, CaCl₂) 2, MgCl2 1, HEPES 10, pH 7.4 with NaOH.

Chloride-free bath solution: Chloride salts in Bath Solution #1 (above) are substituted with gluconate salts.

4. Cell Culture

NIH3T3 mouse fibroblasts stably expressing F508del are used for optical measurements of membrane potential. The cells are maintained at 37° C. in 5% CO₂ and 90% humidity in Dulbecco's modified Eagle's medium supplemented with 2 mM glutamine, 10% fetal bovine serum, 1× NEAA, b-ME, 1×pen/strep, and 25 mM HEPES in 175 cm² culture flasks. For all optical assays, the cells were seeded at ˜20,000/well in 384-well Matrigel-coated plates. For the correction assays, the cells are cultured at 37° C. with and without compounds for 16-24 hours.

### B. Enteroid Assay

1. Solutions

Base medium (ADF+++) consisted of Advanced DMEM/Ham's F12, 2 mM Glutamax, 10 mM HEPES, 1 μg/mL penicillin/streptomycin.

Intestinal enteroid maintenance medium (IEMM) consisted of ADF+++, 1× B27 supplement, 1× N2 supplement, 1.25 mM N-acetyl cysteine, 10 mM Nicotinamide, 50 ng/mL hEGF, 10 nM Gastrin, 1 μg/mL hR-spondin-1, 100 ng/mL hNoggin, TGF-b type 1 inhibitor A-83-01, 100 μg/mL Primocin, 10 μM P38 MAPK inhibitor SB202190.

Bath 1 Buffer consisted of 1 mM MgCl2, 160 mM NaCl, 4.5 mM KCl, 10 mM HEPES, 10 mM Glucose, 2 mM CaCl₂).

Chloride Free Buffer consisted of 1 mM Magnesium Gluconate, 2 mM Calcium Gluconate, 4.5 mM Potassium Gluconate, 160 mM Sodium Gluconate, 10 mM HEPES, 10 mM Glucose.

BathI Dye Solution consisted of Bath 1 Buffer, 0.04% Pluronic F127, 20 μM Methyl Oxonol, 30 μM CaCCinh-A01, 30 μM Chicago Sky Blue.

Chloride Free Dye Solution consisted of Chloride Free Buffer, 0.04% Pluronic F127, 20 μM Methyl Oxonol, 30 μM CaCCinh-A01, 30 μM Chicago Sky Blue.

Chloride Free Dye Stimulation Solution consisted of Chloride Free Dye Solution, 10 μM forskolin, 100 μM IBMX, and 300 nM Compound III.

2. Cell Culture

Human intestinal epithelial enteroid cells were obtained from the Hubrecht Institute for Developmental Biology and Stem Cell Research, Utrecht, The Netherlands and expanded in T-Flasks as previously described (Dekkers J F, Wiegerinck C L, de Jonge H R, Bronsveld I, Janssens H M, de Winter-de Groot K M, Brandsma A M, de Jong N W M, Bijvelds M J C, Scholte B J, Nieuwenhuis E E S, van den Brink S, Clevers H, van der Ent C K, Middendorp S and M Beekman J M. A functional CFTR assay using primary cystic fibrosis intestinal organoids. Nat Med. 2013 July;19(7):939-45.).

3. Enteroid Cell Harvesting and Seeding

Cells were recovered in cell recovery solution, collected by centrifugation at 650 rpm for 5 min at 4° C., resuspended in TrypLE and incubated for 5 min at 37° C. Cells were then collected by centrifugation at 650 rpm for 5 min at 4° C. and resuspended in IEMM containing 10 μM ROCK inhibitor (RI). The cell suspension was passed through a 40 μm cell strainer and resuspended at 1×106 cells/mL in IEMM containing 10 μM RI. Cells were seeded at 5000 cells/well into multi-well plates and incubated for overnight at 37° C., 95% humidity and 5% CO₂ prior to assay.

4. Membrane Potential Dye, Enteroid Assay A

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added, and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 3 μM N-[(6-amino-2-pyridyl)sulfonyl]-6-(3-fluoro-5-isobutoxy-phenyl)-2-[(4S)-2,2,4-trimethylpyrrolidin-1-yl]pyridine-3-carboxamide, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

5. Membrane Potential Dye, Enteroid Assay B

Enteroid cells were incubated with test compound in IEMM for 18-24 hours at 37° C., 95% humidity and 5% CO₂. Following compound incubations, a membrane potential dye assay was employed using a FLIPR Tetra to directly measure the potency and efficacy of the test compound on CFTR-mediated chloride transport following acute addition of 10 μM forskolin and 300 nM N-[2,4-bis(1,1-dimethylethyl)-5-hydroxyphenyl]-1,4-dihydro-4-oxoquinoline-3-carboxamide. Briefly, cells were washed 5 times in Bath 1 Buffer. Bath 1 Dye Solution was added and the cells were incubated for 25 min at room temperature. Following dye incubation, cells were washed 3 times in Chloride Free Dye Solution. Chloride transport was initiated by addition of Chloride Free Dye Stimulation Solution and the fluorescence signal was read for 15 min. The CFTR-mediated chloride transport for each condition was determined from the AUC of the fluorescence response to acute forskolin and 300 nM N-[2,4-bis(1,1-dimethylethyl)-5-hydroxyphenyl]-1,4-dihydro-4-oxoquinoline-3-carboxamide stimulation. Chloride transport was then expressed as a percentage of the chloride transport following treatment with 1 μM (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, 3 μM (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide and 300 nM acute N-[2,4-bis(1,1-dimethylethyl)-5-hydroxyphenyl]-1,4-dihydro-4-oxoquinoline-3-carboxamide triple combination control (% Activity).

C. HBE Assay

1. Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 K₂HPO₄, 3.3 KH₂PO₄, 1.2 MgCl2, 1.2 CaCl₂), 10 Glucose, 10 HEPES (pH adjusted to 7.4 with NaOH) and the apical solution contained (in mM) 145 NaGluconate, 1.2 MgCl2, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

2. Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 1 μM Ivacaftor or were incubated with the corrector compounds at a single fixed concentration of 10 μM in combination with 1 μM Ivacaftor for 18-24 hours at 37° C. and in the presence of 20% human serum. The concentration of corrector compounds with 1 μM Ivacaftor during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, in combination with 18 μM Tezacaftor and 1 μM Ivacaftor.

3. Biological Activity Data

Table 6 represents CFTR modulating activity for representative compounds of the invention generated using one or more of the assays disclosed herein (EC₅₀: +++ is <1 μM; ++ is 1-<3 μM; + is 3-<30 μM; and ND is “not detected in this assay.” % Activity: +++ is >60%; ++ is 30-60%; + is <30%).

TABLE 6

| | | | 3T3 | Ent. | Ent. A | | Ent. B |
|---|---|---|---|---|---|---|---|
| | | 3T3 | Max | A | Max | Ent. B | Max |
| Cmpd | | EC₅₀ | Activity | EC₅₀ | Activity | EC₅₀ | Activity |
| Number | Structure | (μM) | (%) | (μM) | (%) | (μM) | (%) |
| 516 | 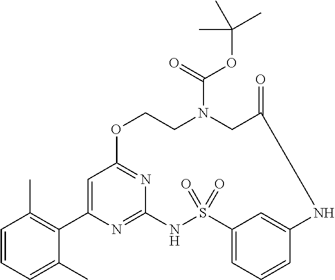 [SMILES: Cc1cccc(C)c1-c1cc2nc(n1)NS(=O)(=O)c1cccc(c1)NC(=O)CN(C(=O)OC(C)(C)C)CCO2] | +++ | +++ | + | +++ | | |
| 5 | 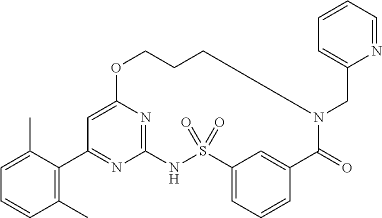 [SMILES: Cc1cccc(C)c1-c1cc2nc(n1)NS(=O)(=O)c1cccc(c1)C(=O)N(Cc1ccccn1)CCCO2] | | | ++ | +++ | | |
| 4 | 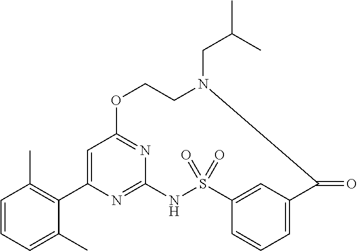 [SMILES: Cc1cccc(C)c1-c1cc2nc(n1)NS(=O)(=O)c1cccc(c1)C(=O)N(CC(C)C)CCO2] | +++ | +++ | +++ | +++ | | |
| 979 | 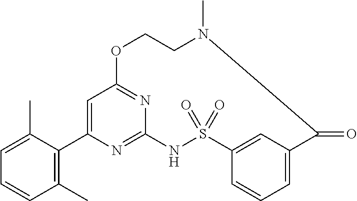 [SMILES: Cc1cccc(C)c1-c1cc2nc(n1)NS(=O)(=O)c1cccc(c1)C(=O)N(C)CCO2] | | | ND | + | | |
| 978 | 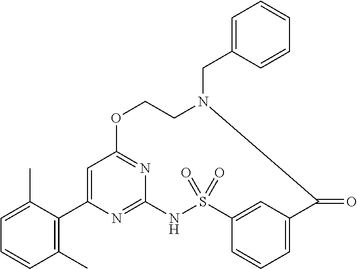 [SMILES: Cc1cccc(C)c1-c1cc2nc(n1)NS(=O)(=O)c1cccc(c1)C(=O)N(Cc1ccccc1)CCO2] | | | +++ | +++ | | |
| 71 | 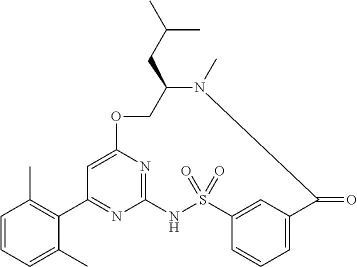 [SMILES: Cc1cccc(C)c1-c1cc2nc(n1)NS(=O)(=O)c1cccc(c1)C(=O)N(C)[C@H](CC(C)C)CO2] | +++ | +++ | +++ | +++ | | |
| 865 | 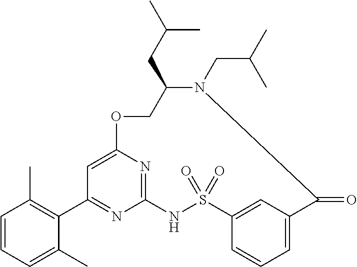 [SMILES: Cc1cccc(C)c1-c1cc2nc(n1)NS(=O)(=O)c1cccc(c1)C(=O)N(CC(C)C)[C@H](CC(C)C)CO2] | +++ | +++ | +++ | +++ | | |
| 977 | 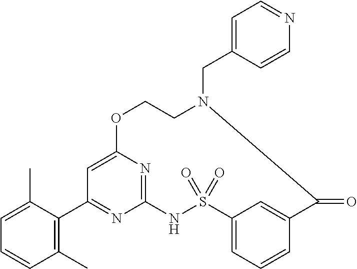 [SMILES: Cc1cccc(C)c1-c1cc2nc(n1)NS(=O)(=O)c1cccc(c1)C(=O)N(Cc1ccncc1)CCO2] | | | + | +++ | | |
| 864 | 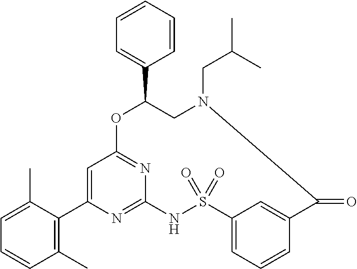 [SMILES: Cc1cccc(C)c1-c1cc2nc(n1)NS(=O)(=O)c1cccc(c1)C(=O)N(CC(C)C)C[C@H](c1ccccc1)O2] | | | +++ | ++ | | |
| 863 | 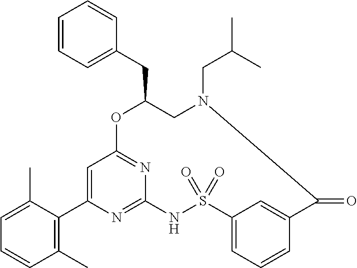 [SMILES: Cc1cccc(C)c1-c1cc2nc(n1)NS(=O)(=O)c1cccc(c1)C(=O)N(CC(C)C)C[C@H](Cc1ccccc1)O2] | | | +++ | +++ | | |
| 18 | 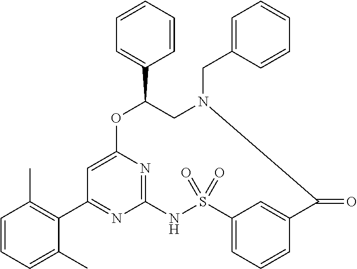 [SMILES: Cc1cccc(C)c1-c1cc2nc(n1)NS(=O)(=O)c1cccc(c1)C(=O)N(Cc1ccccc1)C[C@H](c1ccccc1)O2] | +++ | +++ | +++ | +++ | | |
| 106 | 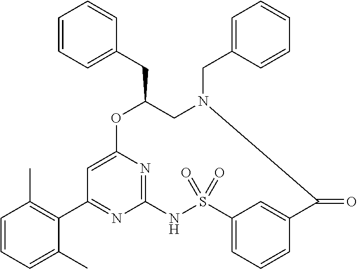 [SMILES: Cc1cccc(C)c1-c1cc2nc(n1)NS(=O)(=O)c1cccc(c1)C(=O)N(Cc1ccccc1)C[C@H](Cc1ccccc1)O2] | +++ | +++ | +++ | +++ | | |
| 862 | 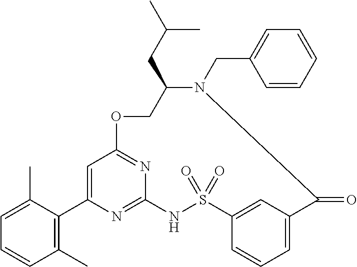 [SMILES: Cc1cccc(C)c1-c1cc2nc(n1)NS(=O)(=O)c1cccc(c1)C(=O)N(Cc1ccccc1)[C@H](CC(C)C)CO2] | | | +++ | +++ | | |
| 1193 | 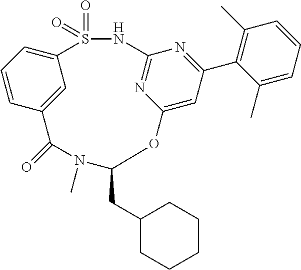 [SMILES: Cc1cccc(C)c1-c1cc2nc(n1)NS(=O)(=O)c1cccc(c1)C(=O)N(C)[C@H](CC1CCCCC1)O2] | +++ | +++ | +++ | +++ | | |
| 44 | 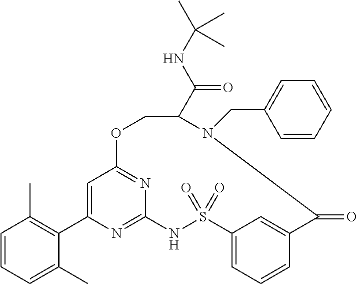 [SMILES: Cc1cccc(C)c1-c1cc2nc(n1)NS(=O)(=O)c1cccc(c1)C(=O)N(Cc1ccccc1)C(C(=O)NC(C)(C)C)CO2] | | | + | ++ | | |
| 515 | 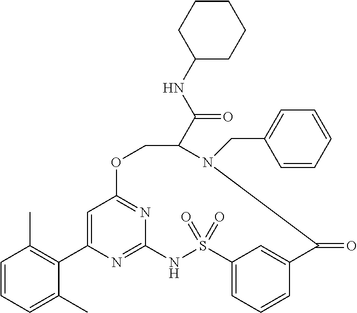 [SMILES: Cc1cccc(C)c1-c1cc2nc(n1)NS(=O)(=O)c1cccc(c1)C(=O)N(Cc1ccccc1)C(C(=O)NC1CCCCC1)CO2] | | | ++ | + | | |
| 514 | 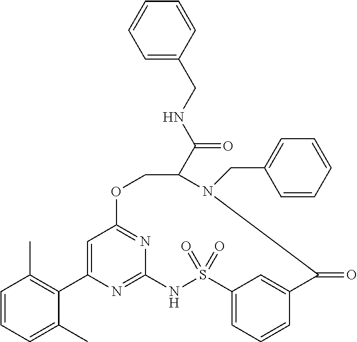 [SMILES: Cc1cccc(C)c1-c1cc2nc(n1)NS(=O)(=O)c1cccc(c1)C(=O)N(Cc1ccccc1)C(C(=O)NCc1ccccc1)CO2] | | | + | + | | |
| 369 | 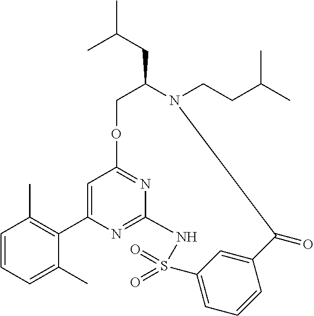 [SMILES: Cc1cccc(C)c1-c1cc2nc(n1)NS(=O)(=O)c1cccc(c1)C(=O)N(CCC(C)C)[C@H](CC(C)C)CO2] | | | +++ | +++ | | |
| 78 | 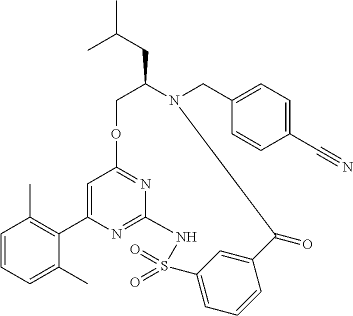 [SMILES: Cc1cccc(C)c1-c1cc2nc(n1)NS(=O)(=O)c1cccc(c1)C(=O)N(Cc1ccc(C#N)cc1)[C@H](CC(C)C)CO2] | +++ | +++ | +++ | +++ | | |
| 79 | 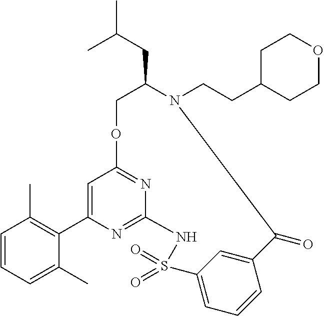 [SMILES: Cc1cccc(C)c1-c1cc2nc(n1)NS(=O)(=O)c1cccc(c1)C(=O)N(CCC1CCOCC1)[C@H](CC(C)C)CO2] | +++ | +++ | +++ | +++ | | |
| 352 | 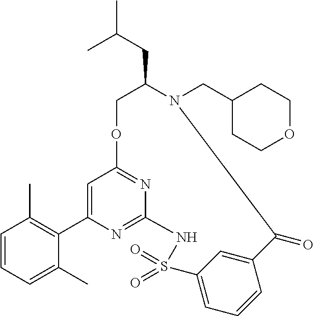 [SMILES: Cc1cccc(C)c1-c1cc2nc(n1)NS(=O)(=O)c1cccc(c1)C(=O)N(CC1CCOCC1)[C@H](CC(C)C)CO2] | | | +++ | +++ | | |
| 351 | 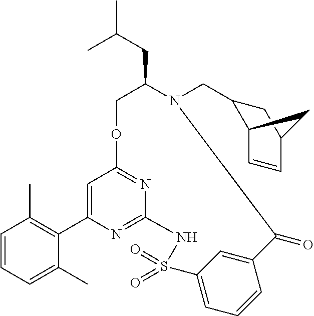 [SMILES: Cc1cccc(C)c1-c1cc2nc(n1)NS(=O)(=O)c1cccc(c1)C(=O)N(CC1CC3C=C[C@@H]1C3)[C@H](CC(C)C)CO2] | | | +++ | +++ | | |
| 368 | 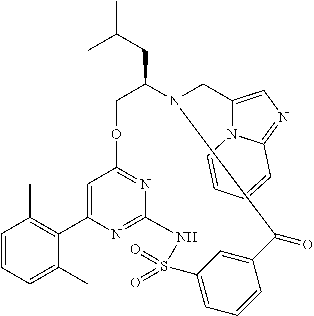 [SMILES: Cc1cccc(C)c1-c1cc2nc(n1)NS(=O)(=O)c1cccc(c1)C(=O)N(Cc1cnc3ccccn13)[C@H](CC(C)C)CO2] | | | +++ | +++ | | |
| 367 | 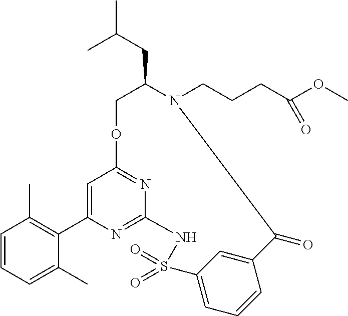 [SMILES: COC(=O)CCCN1C(=O)c2cccc(c2)S(=O)(=O)Nc2nc(cc(-c3c(C)cccc3C)n2)OC[C@H]1CC(C)C] | | | +++ | +++ | | |
| 366 | 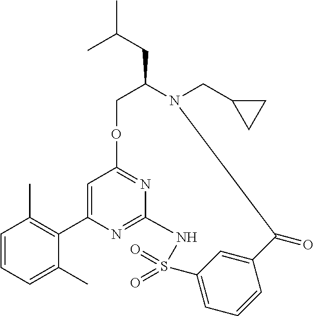 [SMILES: Cc1cccc(C)c1-c1cc2nc(n1)NS(=O)(=O)c1cccc(c1)C(=O)N(CC1CC1)[C@H](CC(C)C)CO2] | | | +++ | +++ | | |
| 365 | 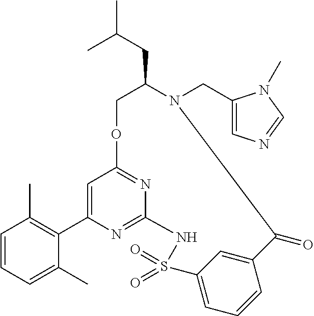 [SMILES: Cc1cccc(C)c1-c1cc2nc(n1)NS(=O)(=O)c1cccc(c1)C(=O)N(Cc1cncn1C)[C@H](CC(C)C)CO2] | | | ++ | +++ | | |
| 364 | 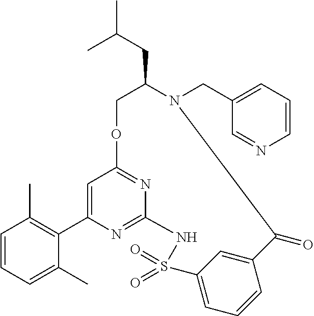 [SMILES: Cc1cccc(C)c1-c1cc2nc(n1)NS(=O)(=O)c1cccc(c1)C(=O)N(Cc1cccnc1)[C@H](CC(C)C)CO2] | | | +++ | +++ | | |
| 77 | 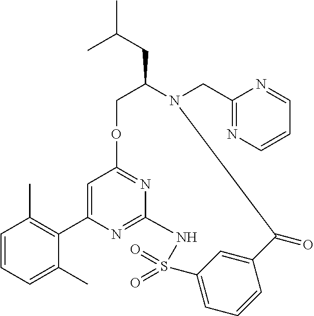 [SMILES: Cc1cccc(C)c1-c1cc2nc(n1)NS(=O)(=O)c1cccc(c1)C(=O)N(Cc1ncccn1)[C@H](CC(C)C)CO2] | | | +++ | +++ | +++ | +++ |
| 363 | 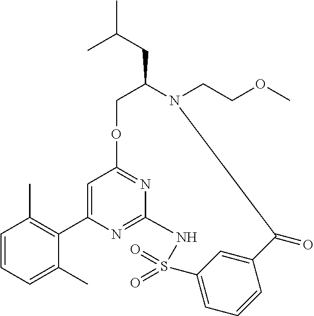 [SMILES: COCCN1C(=O)c2cccc(c2)S(=O)(=O)Nc2nc(cc(-c3c(C)cccc3C)n2)OC[C@H]1CC(C)C] | | | +++ | +++ | +++ | +++ |
| 362 | 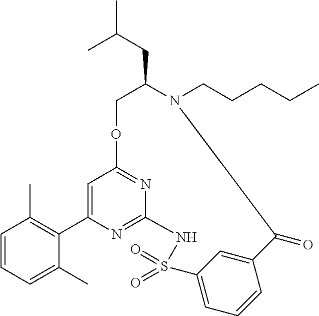 [SMILES: CCCCCN1C(=O)c2cccc(c2)S(=O)(=O)Nc2nc(cc(-c3c(C)cccc3C)n2)OC[C@H]1CC(C)C] | +++ | +++ | +++ | +++ | | |
| 361 | 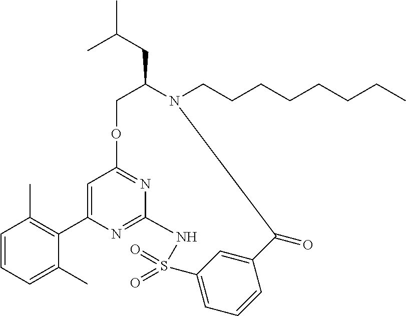 [SMILES: CCCCCCCCN1C(=O)c2cccc(c2)S(=O)(=O)Nc2nc(cc(-c3c(C)cccc3C)n2)OC[C@H]1CC(C)C] | +++ | +++ | +++ | +++ | | |
| 1 | 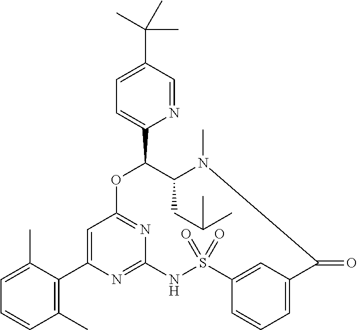 [SMILES: Cc1cccc(C)c1-c1cc2nc(n1)NS(=O)(=O)c1cccc(c1)C(=O)N(C)[C@H](CC(C)C)[C@H](c1ccc(C(C)(C)C)cn1)O2] | +++ | +++ | +++ | +++ | | |
| 2 | 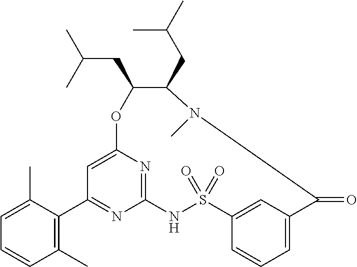 [SMILES: Cc1cccc(C)c1-c1cc2nc(n1)NS(=O)(=O)c1cccc(c1)C(=O)N(C)[C@H](CC(C)C)[C@H](CC(C)C)O2] | +++ | +++ | +++ | +++ | | |
| 97 | 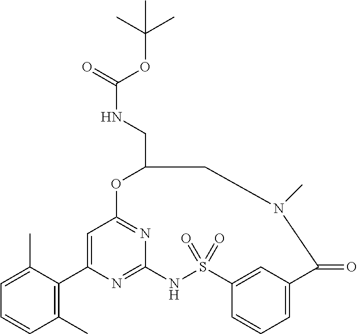 [SMILES: Cc1cccc(C)c1-c1cc2nc(n1)NS(=O)(=O)c1cccc(c1)C(=O)N(C)CC(CNC(=O)OC(C)(C)C)O2] | | | + | ++ | | |
| 346 | 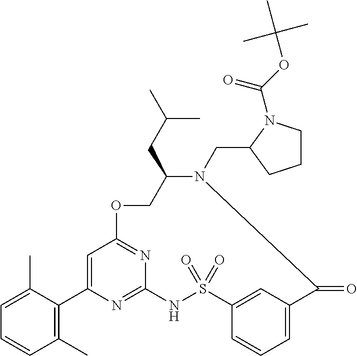 [SMILES: Cc1cccc(C)c1-c1cc2nc(n1)NS(=O)(=O)c1cccc(c1)C(=O)N(CC1CCCN1C(=O)OC(C)(C)C)[C@H](CC(C)C)CO2] | | | +++ | +++ | | |
| 98 | 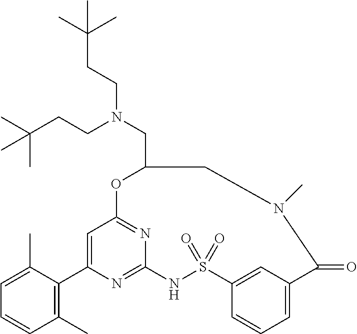 [SMILES: Cc1cccc(C)c1-c1cc2nc(n1)NS(=O)(=O)c1cccc(c1)C(=O)N(C)CC(CN(CCC(C)(C)C)CCC(C)(C)C)O2] | | | +++ | +++ | | |
| 1019 | 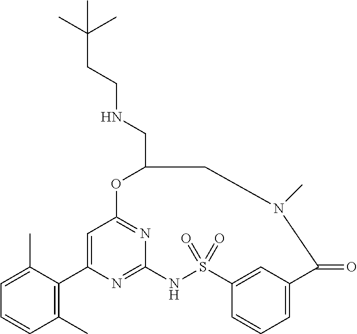 [SMILES: Cc1cccc(C)c1-c1cc2nc(n1)NS(=O)(=O)c1cccc(c1)C(=O)N(C)CC(CNCCC(C)(C)C)O2] | | | ND | + | | |
| 344 | 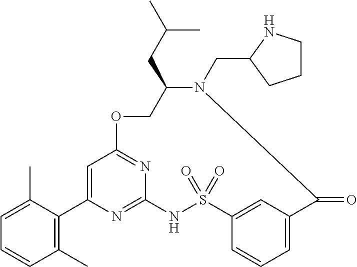 [SMILES: Cc1cccc(C)c1-c1cc2nc(n1)NS(=O)(=O)c1cccc(c1)C(=O)N(CC1CCCN1)[C@H](CC(C)C)CO2] | | | +++ | +++ | | |
| 359 | 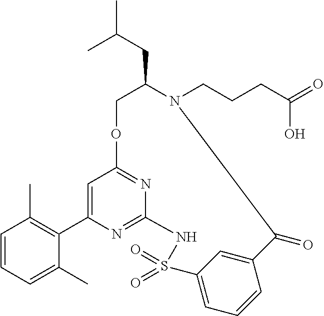 [SMILES: Cc1cccc(C)c1-c1cc2nc(n1)NS(=O)(=O)c1cccc(c1)C(=O)N(CCCC(=O)O)[C@H](CC(C)C)CO2] | | | + | ++ | | |
| 357 | 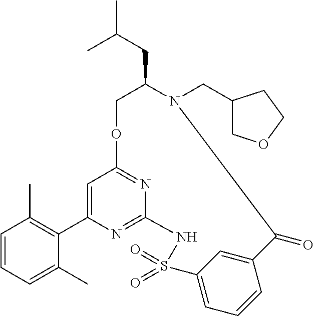 [SMILES: Cc1cccc(C)c1-c1cc2nc(n1)NS(=O)(=O)c1cccc(c1)C(=O)N(CC1CCOC1)[C@H](CC(C)C)CO2] | | | +++ | +++ | | |
| 348 | 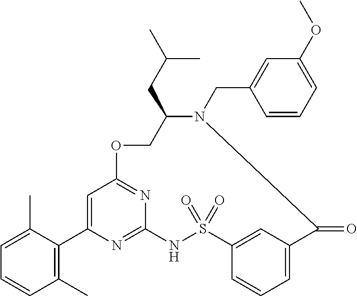 [SMILES: COc1cccc(CN2C(=O)c3cccc(c3)S(=O)(=O)Nc3nc(cc(-c4c(C)cccc4C)n3)OC[C@H]2CC(C)C)c1] | | | +++ | +++ | | |
| 345 | 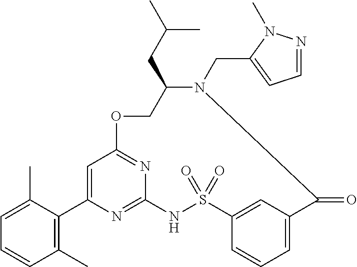 [SMILES: Cc1cccc(C)c1-c1cc2nc(n1)NS(=O)(=O)c1cccc(c1)C(=O)N(Cc1ccnn1C)[C@H](CC(C)C)CO2] | | | +++ | +++ | | |
| 358 | 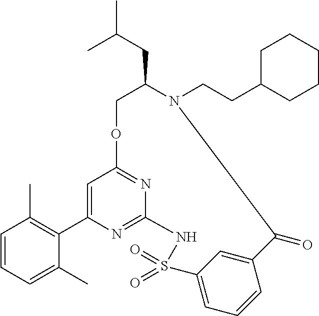 [SMILES: Cc1cccc(C)c1-c1cc2nc(n1)NS(=O)(=O)c1cccc(c1)C(=O)N(CCC1CCCCC1)[C@H](CC(C)C)CO2] | | | +++ | +++ | | |
| 350 | 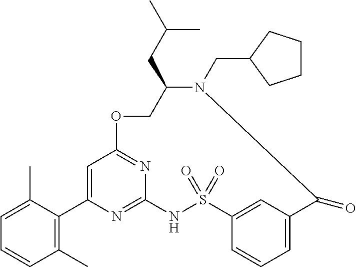 [SMILES: Cc1cccc(C)c1-c1cc2nc(n1)NS(=O)(=O)c1cccc(c1)C(=O)N(CC1CCCC1)[C@H](CC(C)C)CO2] | | | +++ | +++ | | |
| 347 | 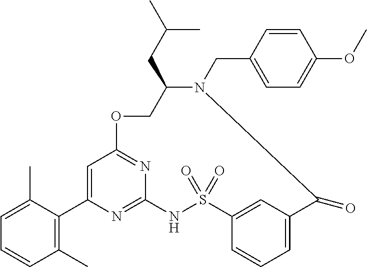 [SMILES: COc1ccc(CN2C(=O)c3cccc(c3)S(=O)(=O)Nc3nc(cc(-c4c(C)cccc4C)n3)OC[C@H]2CC(C)C)cc1] | +++ | +++ | +++ | +++ | | |
| 349 | 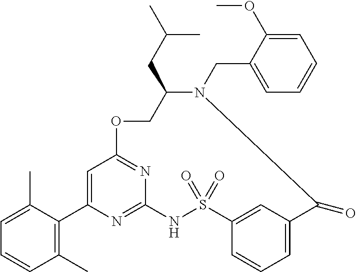 [SMILES: COc1ccccc1CN1C(=O)c2cccc(c2)S(=O)(=O)Nc2nc(cc(-c3c(C)cccc3C)n2)OC[C@H]1CC(C)C] | | | +++ | +++ | | |
| 355 | 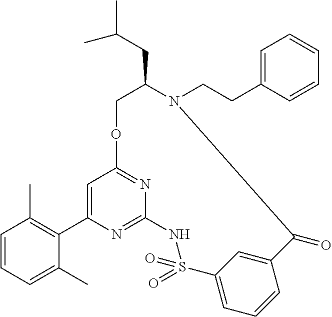 [SMILES: Cc1cccc(C)c1-c1cc2nc(n1)NS(=O)(=O)c1cccc(c1)C(=O)N(CCc1ccccc1)[C@H](CC(C)C)CO2] | | | +++ | +++ | | |
| 356 | 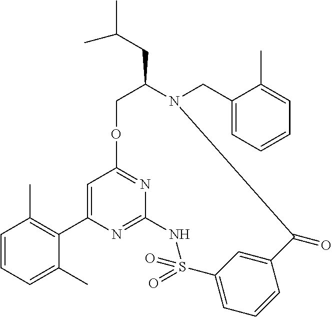 [SMILES: Cc1ccccc1CN1C(=O)c2cccc(c2)S(=O)(=O)Nc2nc(cc(-c3c(C)cccc3C)n2)OC[C@H]1CC(C)C] | | | +++ | +++ | | |
| 360 | 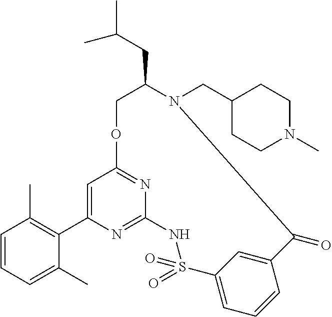 [SMILES: Cc1cccc(C)c1-c1cc2nc(n1)NS(=O)(=O)c1cccc(c1)C(=O)N(CC1CCN(C)CC1)[C@H](CC(C)C)CO2] | | | ++ | +++ | | |
| 991 | 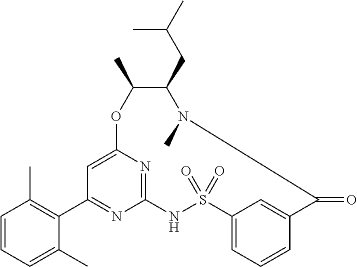 | | | +++ | +++ | | |
| 354 | 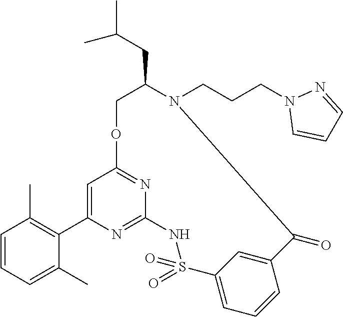 [SMILES: Cc1cccc(C)c1-c1cc2nc(n1)NS(=O)(=O)c1cccc(c1)C(=O)N(CCCn1cccn1)[C@H](CC(C)C)CO2] | | | +++ | +++ | | |
| 88 | 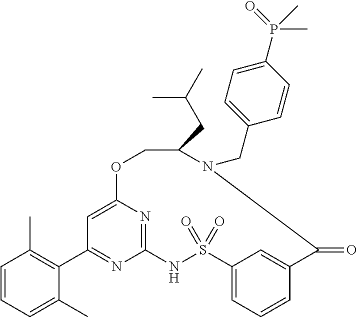 [SMILES: Cc1cccc(C)c1-c1cc2nc(n1)NS(=O)(=O)c1cccc(c1)C(=O)N(Cc1ccc(P(C)(C)=O)cc1)[C@H](CC(C)C)CO2] | | | ++ | ++ | | |
| 858 | 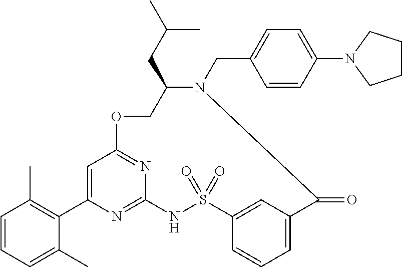 [SMILES: Cc1cccc(C)c1-c1cc2nc(n1)NS(=O)(=O)c1cccc(c1)C(=O)N(Cc1ccc(N3CCCC3)cc1)[C@H](CC(C)C)CO2] | | | +++ | +++ | | |
| 860 | 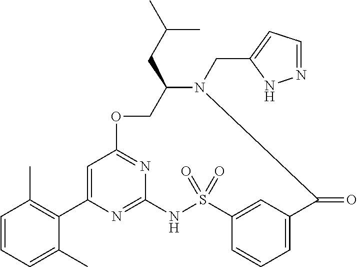 [SMILES: Cc1cccc(C)c1-c1cc2nc(n1)NS(=O)(=O)c1cccc(c1)C(=O)N(Cc1ccn[nH]1)[C@H](CC(C)C)CO2] | | | +++ | +++ | | |
| 81 | 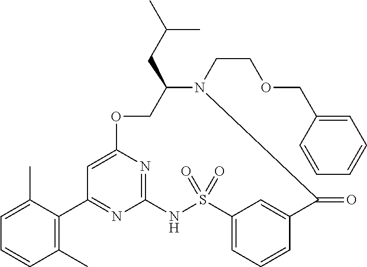 [SMILES: Cc1cccc(C)c1-c1cc2nc(n1)NS(=O)(=O)c1cccc(c1)C(=O)N(CCOCc1ccccc1)[C@H](CC(C)C)CO2] | | | +++ | +++ | | |
| 857 | 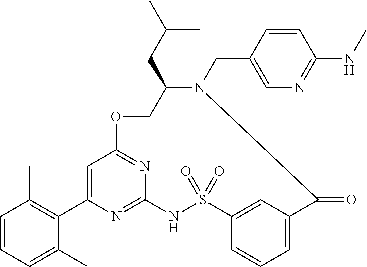 [SMILES: CNc1ccc(CN2C(=O)c3cccc(c3)S(=O)(=O)Nc3nc(cc(-c4c(C)cccc4C)n3)OC[C@H]2CC(C)C)cn1] | | | +++ | +++ | | |
| 861 | 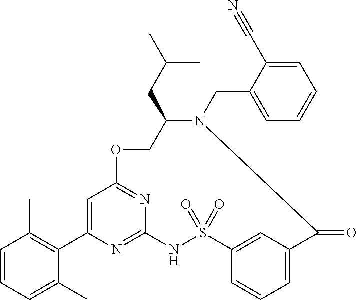 [SMILES: Cc1cccc(C)c1-c1cc2nc(n1)NS(=O)(=O)c1cccc(c1)C(=O)N(Cc1ccccc1C#N)[C@H](CC(C)C)CO2] | | | ++ | ++ | | |
| 859 | 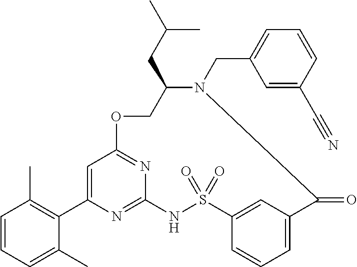 [SMILES: Cc1cccc(C)c1-c1cc2nc(n1)NS(=O)(=O)c1cccc(c1)C(=O)N(Cc1cccc(C#N)c1)[C@H](CC(C)C)CO2] | | | +++ | +++ | | |
| 856 | 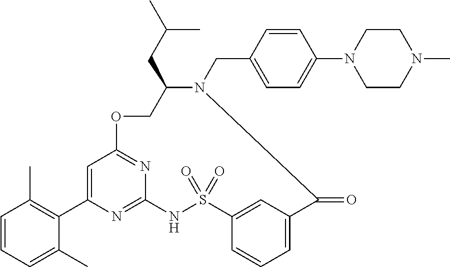 [SMILES: Cc1cccc(C)c1-c1cc2nc(n1)NS(=O)(=O)c1cccc(c1)C(=O)N(Cc1ccc(N3CCN(C)CC3)cc1)[C@H](CC(C)C)CO2] | | | +++ | +++ | | |
| 513 | 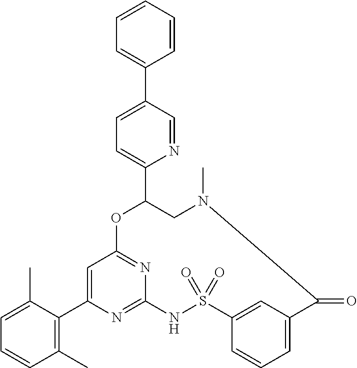 [SMILES: Cc1cccc(C)c1-c1cc2nc(n1)NS(=O)(=O)c1cccc(c1)C(=O)N(C)CC(c1ccc(-c3ccccc3)cn1)O2] | | | ++ | +++ | | |
| 353 | 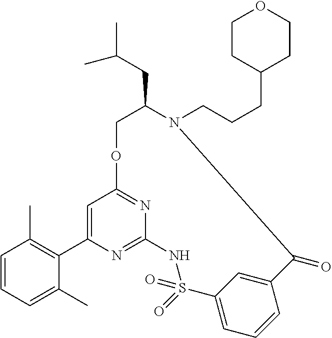 [SMILES: Cc1cccc(C)c1-c1cc2nc(n1)NS(=O)(=O)c1cccc(c1)C(=O)N(CCCC1CCOCC1)[C@H](CC(C)C)CO2] | +++ | +++ | +++ | +++ | | |
| 677 | 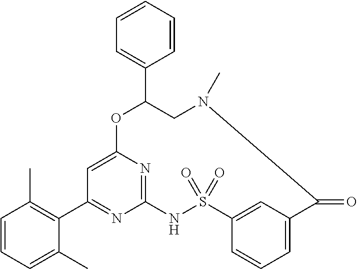 [SMILES: Cc1cccc(C)c1-c1cc2nc(n1)NS(=O)(=O)c1cccc(c1)C(=O)N(C)CC(c1ccccc1)O2] | | | +++ | +++ | | |
| 1109 | 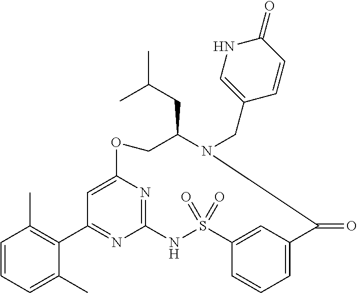 [SMILES: Cc1cccc(C)c1-c1cc2nc(n1)NS(=O)(=O)c1cccc(c1)C(=O)N(Cc1ccc(=O)[nH]c1)[C@H](CC(C)C)CO2] | | | ++ | +++ | | |
| 432 | 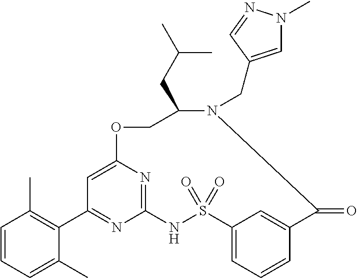 [SMILES: Cc1cccc(C)c1-c1cc2nc(n1)NS(=O)(=O)c1cccc(c1)C(=O)N(Cc1cnn(C)c1)[C@H](CC(C)C)CO2] | | | +++ | +++ | | |
| 430 | 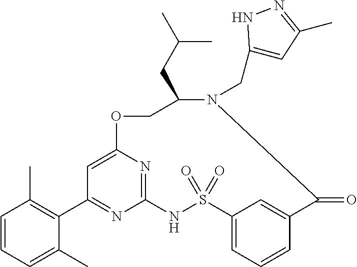 [SMILES: Cc1cc(CN2C(=O)c3cccc(c3)S(=O)(=O)Nc3nc(cc(-c4c(C)cccc4C)n3)OC[C@H]2CC(C)C)[nH]n1] | | | +++ | +++ | | |
| 429 | 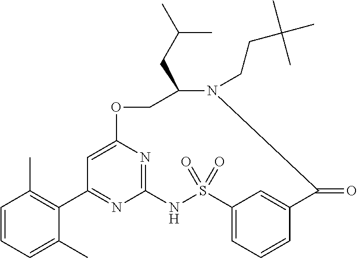 [SMILES: Cc1cccc(C)c1-c1cc2nc(n1)NS(=O)(=O)c1cccc(c1)C(=O)N(CCC(C)(C)C)[C@H](CC(C)C)CO2] | | | +++ | +++ | | |
| 37 | 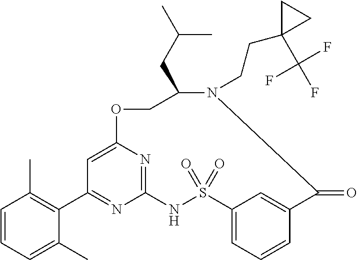 [SMILES: Cc1cccc(C)c1-c1cc2nc(n1)NS(=O)(=O)c1cccc(c1)C(=O)N(CCC1(C(F)(F)F)CC1)[C@H](CC(C)C)CO2] | +++ | +++ | +++ | +++ | | |
| 1108 | 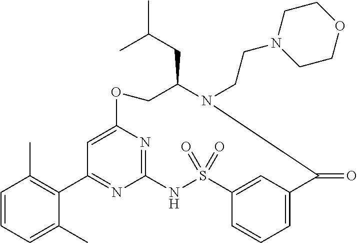 [SMILES: Cc1cccc(C)c1-c1cc2nc(n1)NS(=O)(=O)c1cccc(c1)C(=O)N(CCN1CCOCC1)[C@H](CC(C)C)CO2] | | | +++ | +++ | | |
| 990 | 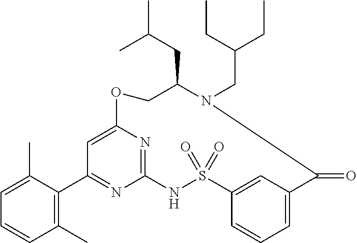 [SMILES: CCC(CC)CN1C(=O)c2cccc(c2)S(=O)(=O)Nc2nc(cc(-c3c(C)cccc3C)n2)OC[C@H]1CC(C)C] | | | +++ | +++ | | |
| 989 | 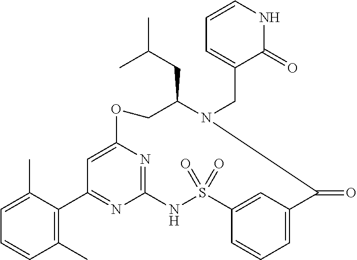 [SMILES: Cc1cccc(C)c1-c1cc2nc(n1)NS(=O)(=O)c1cccc(c1)C(=O)N(Cc1ccc[nH]c1=O)[C@H](CC(C)C)CO2] | | | ++ | +++ | | |
| 855 | 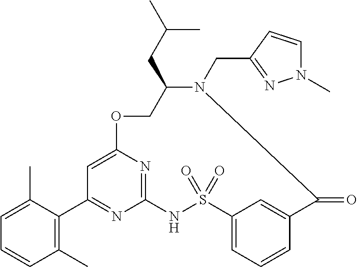 [SMILES: Cc1cccc(C)c1-c1cc2nc(n1)NS(=O)(=O)c1cccc(c1)C(=O)N(Cc1ccn(C)n1)[C@H](CC(C)C)CO2] | | | +++ | +++ | | |
| 667 | 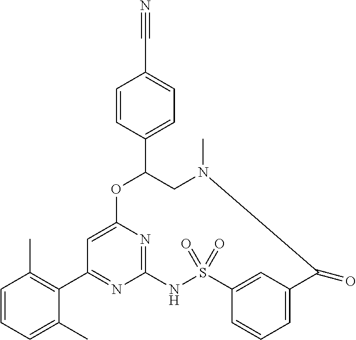 [SMILES: Cc1cccc(C)c1-c1cc2nc(n1)NS(=O)(=O)c1cccc(c1)C(=O)N(C)CC(c1ccc(C#N)cc1)O2] | | | ++ | ++ | | |
| 1016 | 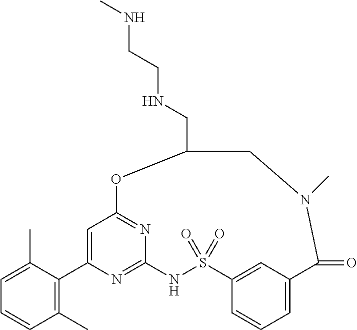 [SMILES: CNCCNCC1CN(C)C(=O)c2cccc(c2)S(=O)(=O)Nc2nc(cc(-c3c(C)cccc3C)n2)O1] | | | ND | + | | |
| 8 | 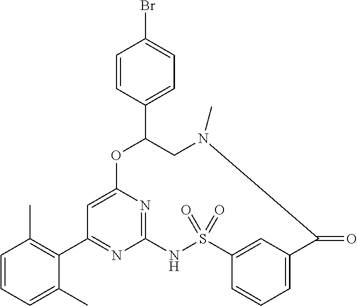 [SMILES: Cc1cccc(C)c1-c1cc2nc(n1)NS(=O)(=O)c1cccc(c1)C(=O)N(C)CC(c1ccc(Br)cc1)O2] | | | +++ | +++ | | |
| 9 | 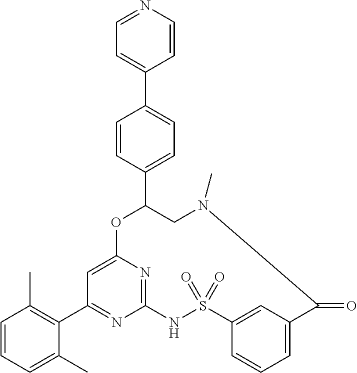 [SMILES: Cc1cccc(C)c1-c1cc2nc(n1)NS(=O)(=O)c1cccc(c1)C(=O)N(C)CC(c1ccc(-c3ccncc3)cc1)O2] | | | +++ | ++ | | |
| 511 | 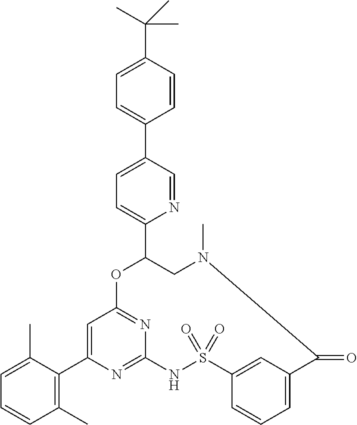 [SMILES: Cc1cccc(C)c1-c1cc2nc(n1)NS(=O)(=O)c1cccc(c1)C(=O)N(C)CC(c1ccc(-c3ccc(C(C)(C)C)cc3)cn1)O2] | | | +++ | +++ | | |
| 341 | 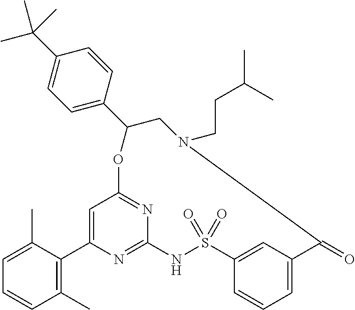 [SMILES: Cc1cccc(C)c1-c1cc2nc(n1)NS(=O)(=O)c1cccc(c1)C(=O)N(CCC(C)C)CC(c1ccc(C(C)(C)C)cc1)O2] | | | ++ | ++ | | |
| 343 | 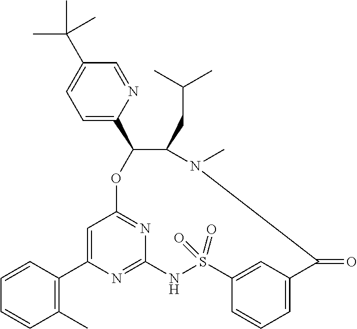 [SMILES: Cc1ccccc1-c1cc2nc(n1)NS(=O)(=O)c1cccc(c1)C(=O)N(C)[C@H](CC(C)C)[C@H](c1ccc(C(C)(C)C)cn1)O2] | | | +++ | +++ | | |
| 428 | 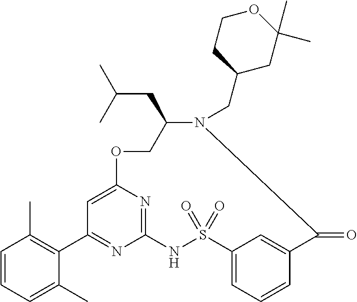 [SMILES: Cc1cccc(C)c1-c1cc2nc(n1)NS(=O)(=O)c1cccc(c1)C(=O)N(C[C@H]1CCOC(C)(C)C1)[C@H](CC(C)C)CO2] | | | +++ | +++ | | |
| 431 | 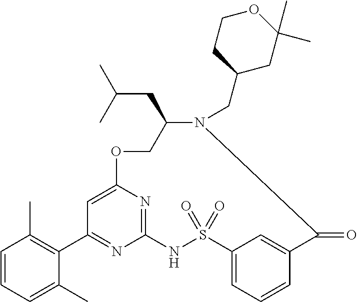 [SMILES: Cc1cccc(C)c1-c1cc2nc(n1)NS(=O)(=O)c1cccc(c1)C(=O)N(C[C@H]1CCOC(C)(C)C1)[C@H](CC(C)C)CO2] | | | +++ | +++ | | |
| 427 | 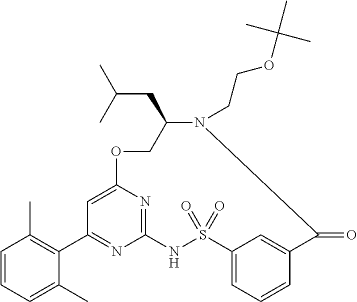 [SMILES: Cc1cccc(C)c1-c1cc2nc(n1)NS(=O)(=O)c1cccc(c1)C(=O)N(CCOC(C)(C)C)[C@H](CC(C)C)CO2] | | | +++ | +++ | | |
| 666 | 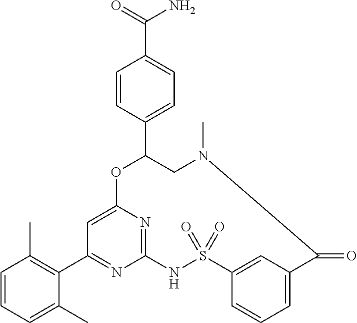 [SMILES: Cc1cccc(C)c1-c1cc2nc(n1)NS(=O)(=O)c1cccc(c1)C(=O)N(C)CC(c1ccc(C(N)=O)cc1)O2] | | | ND | + | | |
| 99 | 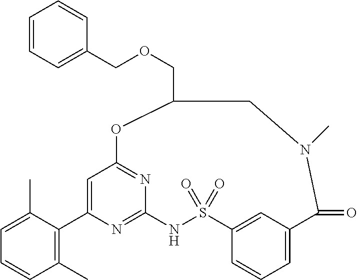 [SMILES: Cc1cccc(C)c1-c1cc2nc(n1)NS(=O)(=O)c1cccc(c1)C(=O)N(C)CC(COCc1ccccc1)O2] | | | ++ | ++ | | |
| 100 | 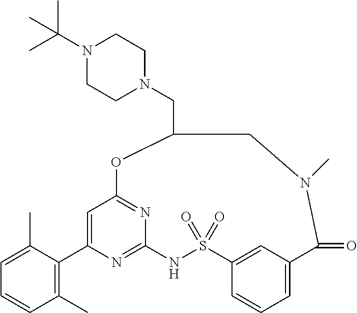 [SMILES: Cc1cccc(C)c1-c1cc2nc(n1)NS(=O)(=O)c1cccc(c1)C(=O)N(C)CC(CN1CCN(C(C)(C)C)CC1)O2] | | | ND | ND | | |
| 1015 | 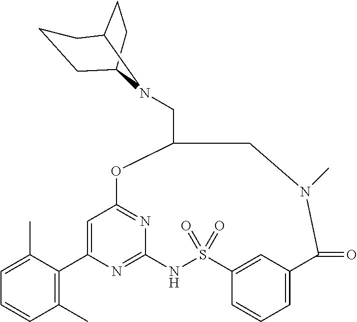 [SMILES: Cc1cccc(C)c1-c1cc2nc(n1)NS(=O)(=O)c1cccc(c1)C(=O)N(C)CC(CN1C3CCC[C@H]1CC3)O2] | | | ND | + | | |
| 1013 | 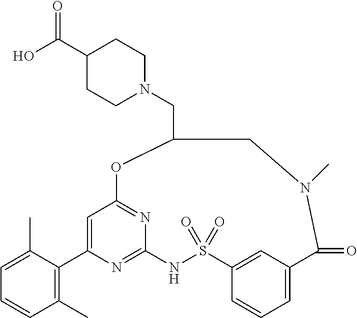 [SMILES: Cc1cccc(C)c1-c1cc2nc(n1)NS(=O)(=O)c1cccc(c1)C(=O)N(C)CC(CN1CCC(C(=O)O)CC1)O2] | | | ND | ND | | |
| 1014 | 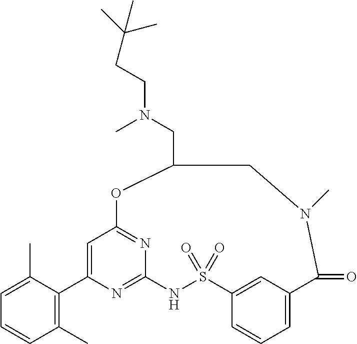 [SMILES: Cc1cccc(C)c1-c1cc2nc(n1)NS(=O)(=O)c1cccc(c1)C(=O)N(C)CC(CN(C)CCC(C)(C)C)O2] | | | + | +++ | | |
| 426 | 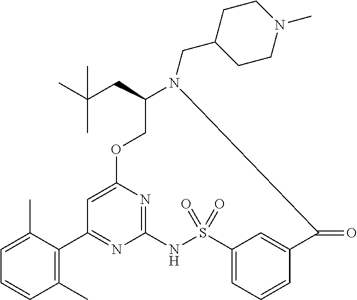 [SMILES: Cc1cccc(C)c1-c1cc2nc(n1)NS(=O)(=O)c1cccc(c1)C(=O)N(CC1CCN(C)CC1)[C@H](CC(C)(C)C)CO2] | | | ++ | +++ | | |
| 40 | 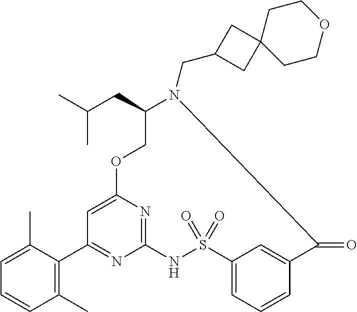 [SMILES: Cc1cccc(C)c1-c1cc2nc(n1)NS(=O)(=O)c1cccc(c1)C(=O)N(CC1CC3(CCOCC3)C1)[C@H](CC(C)C)CO2] | +++ | +++ | +++ | +++ | | |
| 425 | 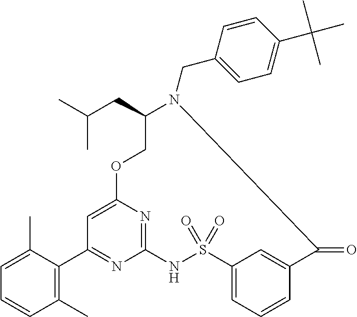 [SMILES: Cc1cccc(C)c1-c1cc2nc(n1)NS(=O)(=O)c1cccc(c1)C(=O)N(Cc1ccc(C(C)(C)C)cc1)[C@H](CC(C)C)CO2] | | | +++ | +++ | | |
| 676 | 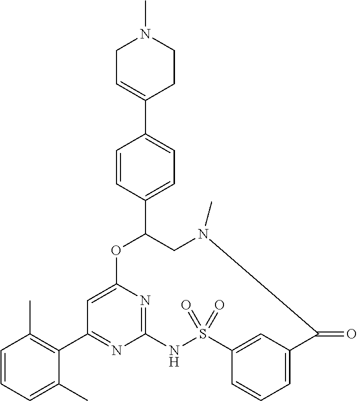 [SMILES: Cc1cccc(C)c1-c1cc2nc(n1)NS(=O)(=O)c1cccc(c1)C(=O)N(C)CC(c1ccc(C3=CCN(C)CC3)cc1)O2] | | | + | ++ | | |
| 675 | 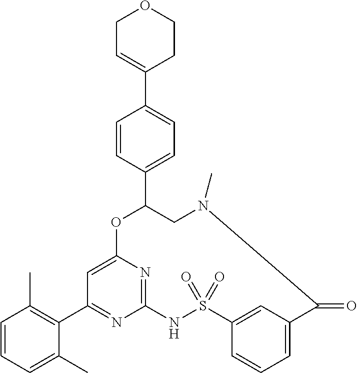 [SMILES: Cc1cccc(C)c1-c1cc2nc(n1)NS(=O)(=O)c1cccc(c1)C(=O)N(C)CC(c1ccc(C3=CCOCC3)cc1)O2] | | | +++ | ++ | | |
| 674 | 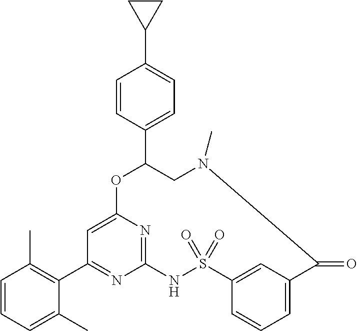 [SMILES: Cc1cccc(C)c1-c1cc2nc(n1)NS(=O)(=O)c1cccc(c1)C(=O)N(C)CC(c1ccc(C3CC3)cc1)O2] | | | +++ | ++ | | |
| 672 | 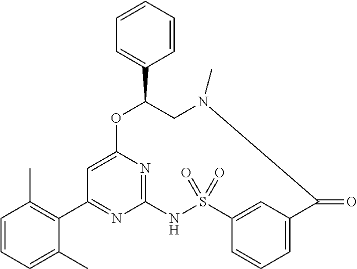 [SMILES: Cc1cccc(C)c1-c1cc2nc(n1)NS(=O)(=O)c1cccc(c1)C(=O)N(C)C[C@H](c1ccccc1)O2] | | | +++ | ++ | | |
| 673 | 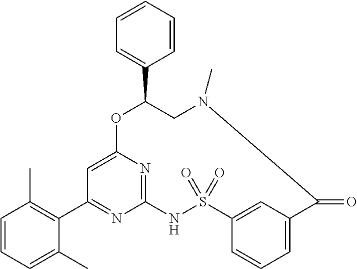 [SMILES: Cc1cccc(C)c1-c1cc2nc(n1)NS(=O)(=O)c1cccc(c1)C(=O)N(C)C[C@H](c1ccccc1)O2] | | | + | + | | |
| 340 | 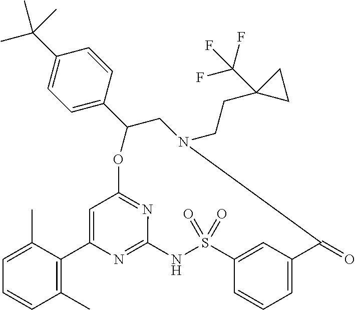 [SMILES: Cc1cccc(C)c1-c1cc2nc(n1)NS(=O)(=O)c1cccc(c1)C(=O)N(CCC1(C(F)(F)F)CC1)CC(c1ccc(C(C)(C)C)cc1)O2] | | | | | | |
| 342 | 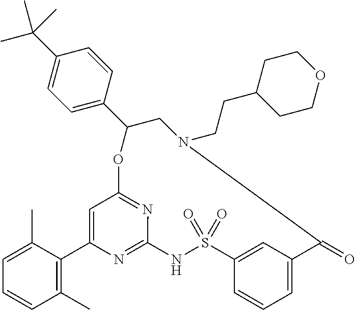 [SMILES: Cc1cccc(C)c1-c1cc2nc(n1)NS(=O)(=O)c1cccc(c1)C(=O)N(CCC1CCOCC1)CC(c1ccc(C(C)(C)C)cc1)O2] | | | +++ | +++ | | |
| 14 | 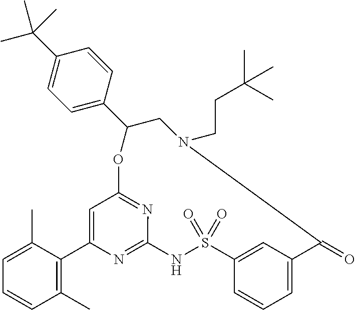 [SMILES: Cc1cccc(C)c1-c1cc2nc(n1)NS(=O)(=O)c1cccc(c1)C(=O)N(CCC(C)(C)C)CC(c1ccc(C(C)(C)C)cc1)O2] | | | +++ | ++ | | |
| 74 | 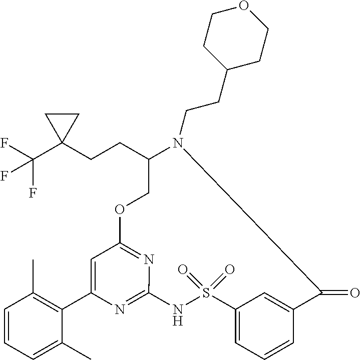 [SMILES: Cc1cccc(C)c1-c1cc2nc(n1)NS(=O)(=O)c1cccc(c1)C(=O)N(CCC1CCOCC1)C(CCC1(C(F)(F)F)CC1)CO2] | | | +++ | +++ | | |
| 688 | 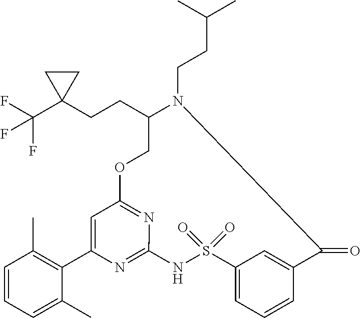 [SMILES: Cc1cccc(C)c1-c1cc2nc(n1)NS(=O)(=O)c1cccc(c1)C(=O)N(CCC(C)C)C(CCC1(C(F)(F)F)CC1)CO2] | | | +++ | +++ | | |
| 689 | 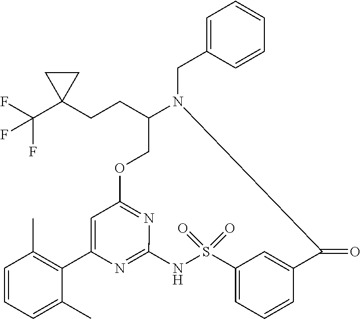 [SMILES: Cc1cccc(C)c1-c1cc2nc(n1)NS(=O)(=O)c1cccc(c1)C(=O)N(Cc1ccccc1)C(CCC1(C(F)(F)F)CC1)CO2] | | | +++ | +++ | | |
| 1017 | 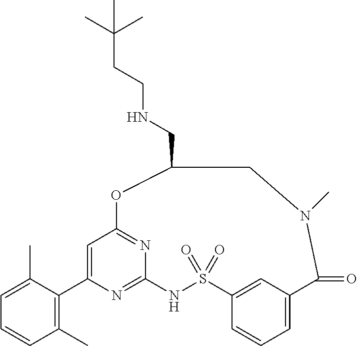 [SMILES: Cc1cccc(C)c1-c1cc2nc(n1)NS(=O)(=O)c1cccc(c1)C(=O)N(C)C[C@H](CNCCC(C)(C)C)O2] | | | ND | + | | |
| 1018 | 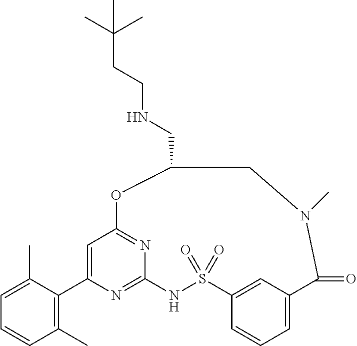 [SMILES: Cc1cccc(C)c1-c1cc2nc(n1)NS(=O)(=O)c1cccc(c1)C(=O)N(C)C[C@@H](CNCCC(C)(C)C)O2] | | | ND | + | | |
| 1012 | 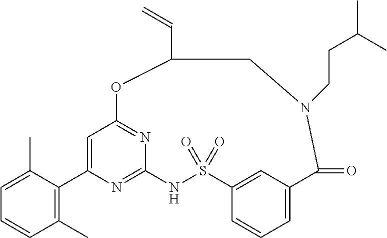 [SMILES: C=CC1CN(CCC(C)C)C(=O)c2cccc(c2)S(=O)(=O)Nc2nc(cc(-c3c(C)cccc3C)n2)O1] | | | +++ | ++ | | |
| 512 | 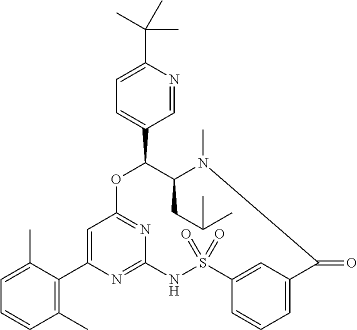 [SMILES: Cc1cccc(C)c1-c1cc2nc(n1)NS(=O)(=O)c1cccc(c1)C(=O)N(C)[C@@H](CC(C)C)[C@H](c1ccc(C(C)(C)C)nc1)O2] | | | ND | + | | |
| 80 | 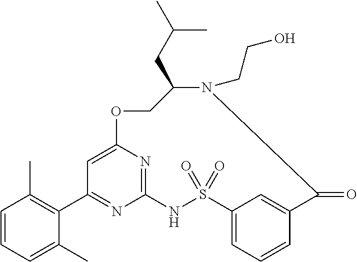 [SMILES: Cc1cccc(C)c1-c1cc2nc(n1)NS(=O)(=O)c1cccc(c1)C(=O)N(CCO)[C@H](CC(C)C)CO2] | +++ | +++ | +++ | +++ | | |
| 38 | 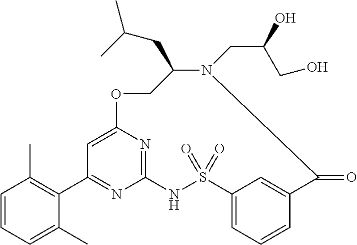 [SMILES: Cc1cccc(C)c1-c1cc2nc(n1)NS(=O)(=O)c1cccc(c1)C(=O)N(C[C@@H](O)CO)[C@H](CC(C)C)CO2] | | | +++ | +++ | | |
| 39 | 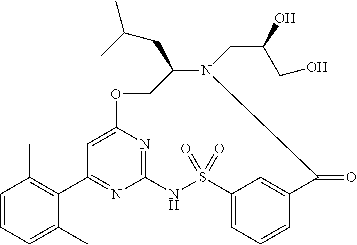 [SMILES: Cc1cccc(C)c1-c1cc2nc(n1)NS(=O)(=O)c1cccc(c1)C(=O)N(C[C@@H](O)CO)[C@H](CC(C)C)CO2] | | | +++ | +++ | | |
| 138 | 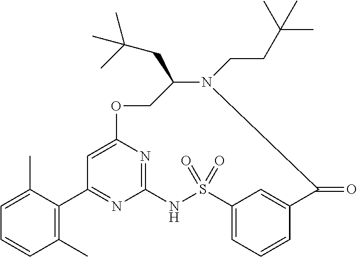 [SMILES: Cc1cccc(C)c1-c1cc2nc(n1)NS(=O)(=O)c1cccc(c1)C(=O)N(CCC(C)(C)C)[C@H](CC(C)(C)C)CO2] | | | +++ | +++ | | |
| 424 | 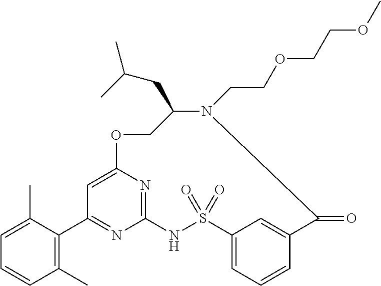 [SMILES: COCCOCCN1C(=O)c2cccc(c2)S(=O)(=O)Nc2nc(cc(-c3c(C)cccc3C)n2)OC[C@H]1CC(C)C] | | | +++ | +++ | | |
| 86 | 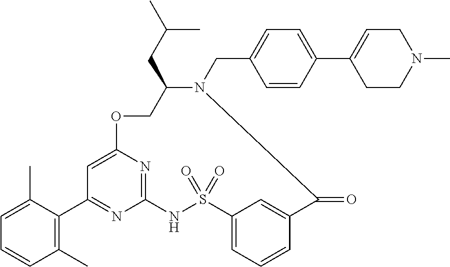 [SMILES: Cc1cccc(C)c1-c1cc2nc(n1)NS(=O)(=O)c1cccc(c1)C(=O)N(Cc1ccc(C3=CCN(C)CC3)cc1)[C@H](CC(C)C)CO2] | | | +++ | +++ | | |
| 854 | 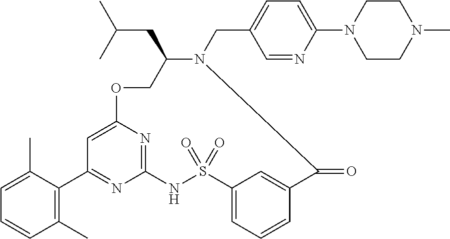 [SMILES: Cc1cccc(C)c1-c1cc2nc(n1)NS(=O)(=O)c1cccc(c1)C(=O)N(Cc1ccc(N3CCN(C)CC3)nc1)[C@H](CC(C)C)CO2] | | | +++ | +++ | | |
| 10 | 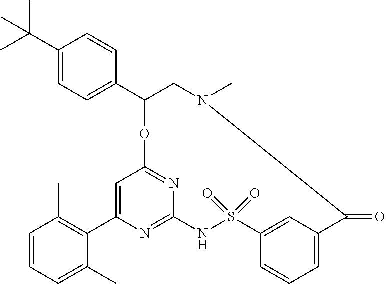 [SMILES: Cc1cccc(C)c1-c1cc2nc(n1)NS(=O)(=O)c1cccc(c1)C(=O)N(C)CC(c1ccc(C(C)(C)C)cc1)O2] | | | +++ | +++ | | |
| 852 | 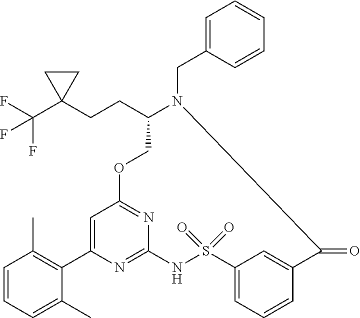 [SMILES: Cc1cccc(C)c1-c1cc2nc(n1)NS(=O)(=O)c1cccc(c1)C(=O)N(Cc1ccccc1)[C@@H](CCC1(C(F)(F)F)CC1)CO2] | | | +++ | ++ | | |
| 853 | 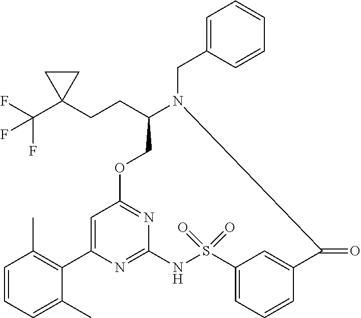 [SMILES: Cc1cccc(C)c1-c1cc2nc(n1)NS(=O)(=O)c1cccc(c1)C(=O)N(Cc1ccccc1)[C@H](CCC1(C(F)(F)F)CC1)CO2] | | | +++ | ++ | | |
| 101 | 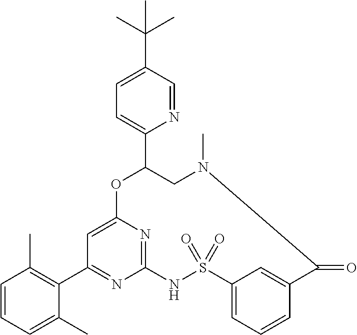 [SMILES: Cc1cccc(C)c1-c1cc2nc(n1)NS(=O)(=O)c1cccc(c1)C(=O)N(C)CC(c1ccc(C(C)(C)C)cn1)O2] | | | +++ | +++ | | |
| 87 | 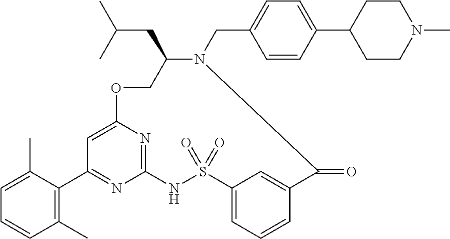 [SMILES: Cc1cccc(C)c1-c1cc2nc(n1)NS(=O)(=O)c1cccc(c1)C(=O)N(Cc1ccc(C3CCN(C)CC3)cc1)[C@H](CC(C)C)CO2] | | | +++ | +++ | | |
| 82 | 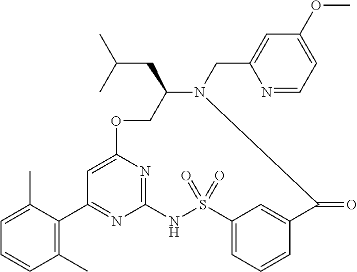 [SMILES: COc1ccnc(CN2C(=O)c3cccc(c3)S(=O)(=O)Nc3nc(cc(-c4c(C)cccc4C)n3)OC[C@H]2CC(C)C)c1] | | | +++ | +++ | | |
| 976 | 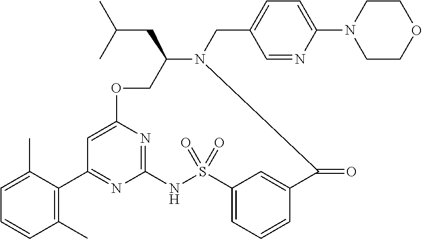 [SMILES: Cc1cccc(C)c1-c1cc2nc(n1)NS(=O)(=O)c1cccc(c1)C(=O)N(Cc1ccc(N3CCOCC3)nc1)[C@H](CC(C)C)CO2] | | | +++ | +++ | | |
| 975 | 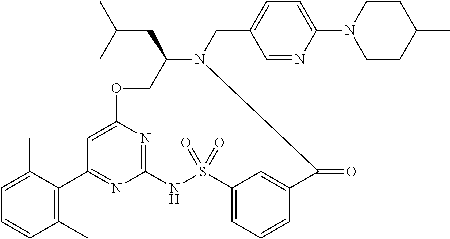 [SMILES: Cc1cccc(C)c1-c1cc2nc(n1)NS(=O)(=O)c1cccc(c1)C(=O)N(Cc1ccc(N3CCC(C)CC3)nc1)[C@H](CC(C)C)CO2] | +++ | +++ | +++ | +++ | +++ | +++ |
| 421 | 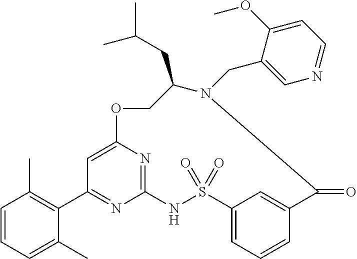 [SMILES: COc1ccncc1CN1C(=O)c2cccc(c2)S(=O)(=O)Nc2nc(cc(-c3c(C)cccc3C)n2)OC[C@H]1CC(C)C] | | | +++ | +++ | | |
| 41 | 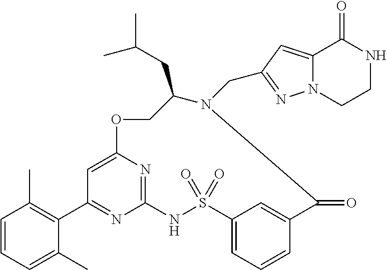 [SMILES: Cc1cccc(C)c1-c1cc2nc(n1)NS(=O)(=O)c1cccc(c1)C(=O)N(Cc1cc3n(n1)CCNC3=O)[C@H](CC(C)C)CO2] | | | +++ | +++ | | |
| 419 | 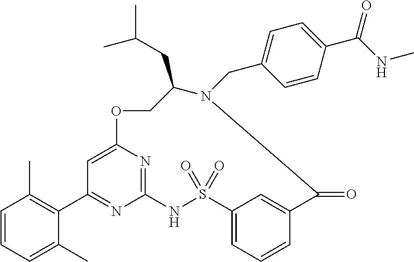 [SMILES: CNC(=O)c1ccc(CN2C(=O)c3cccc(c3)S(=O)(=O)Nc3nc(cc(-c4c(C)cccc4C)n3)OC[C@H]2CC(C)C)cc1] | +++ | +++ | +++ | +++ | | |
| 418 | 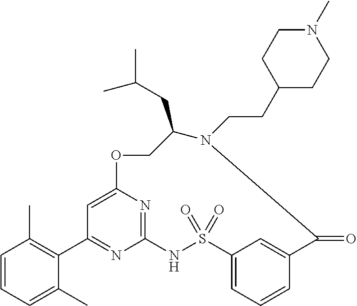 [SMILES: Cc1cccc(C)c1-c1cc2nc(n1)NS(=O)(=O)c1cccc(c1)C(=O)N(CCC1CCN(C)CC1)[C@H](CC(C)C)CO2] | +++ | +++ | +++ | +++ | | |
| 423 | 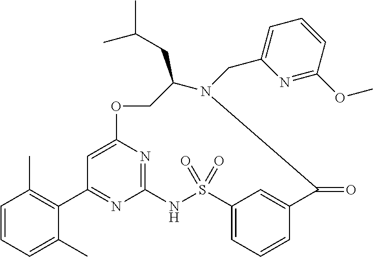 [SMILES: COc1cccc(CN2C(=O)c3cccc(c3)S(=O)(=O)Nc3nc(cc(-c4c(C)cccc4C)n3)OC[C@H]2CC(C)C)n1] | | | +++ | +++ | | |
| 422 | 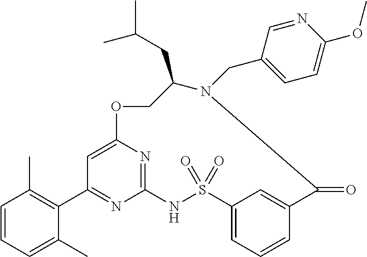 [SMILES: COc1ccc(CN2C(=O)c3cccc(c3)S(=O)(=O)Nc3nc(cc(-c4c(C)cccc4C)n3)OC[C@H]2CC(C)C)cn1] | | | +++ | +++ | | |
| 420 | 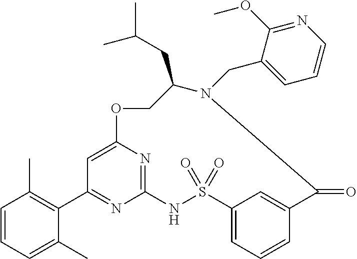 [SMILES: COc1ncccc1CN1C(=O)c2cccc(c2)S(=O)(=O)Nc2nc(cc(-c3c(C)cccc3C)n2)OC[C@H]1CC(C)C] | | | +++ | +++ | | |
| 338 | 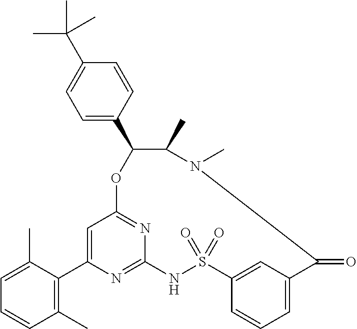 [SMILES: Cc1cccc(C)c1-c1cc2nc(n1)NS(=O)(=O)c1cccc(c1)C(=O)N(C)[C@H](C)[C@H](c1ccc(C(C)(C)C)cc1)O2] | +++ | +++ | ND | + | | |
| 850 | 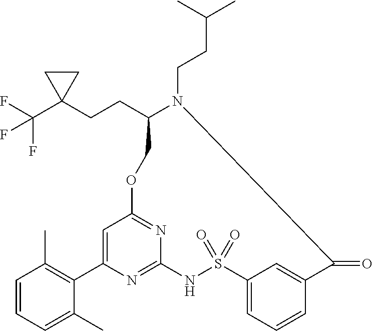 [SMILES: Cc1cccc(C)c1-c1cc2nc(n1)NS(=O)(=O)c1cccc(c1)C(=O)N(CCC(C)C)[C@H](CCC1(C(F)(F)F)CC1)CO2] | +++ | +++ | +++ | +++ | | |
| 851 | 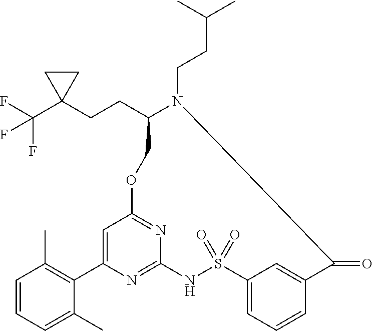 [SMILES: Cc1cccc(C)c1-c1cc2nc(n1)NS(=O)(=O)c1cccc(c1)C(=O)N(CCC(C)C)[C@H](CCC1(C(F)(F)F)CC1)CO2] | | | ND | + | | |
| 72 | 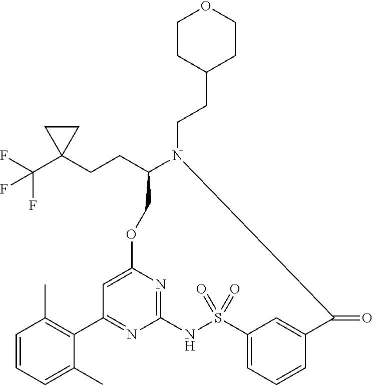 [SMILES: Cc1cccc(C)c1-c1cc2nc(n1)NS(=O)(=O)c1cccc(c1)C(=O)N(CCC1CCOCC1)[C@H](CCC1(C(F)(F)F)CC1)CO2] | +++ | +++ | +++ | +++ | | |
| 73 | 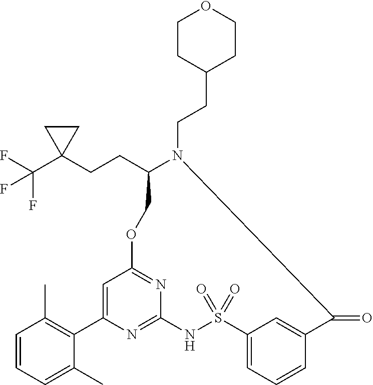 [SMILES: Cc1cccc(C)c1-c1cc2nc(n1)NS(=O)(=O)c1cccc(c1)C(=O)N(CCC1CCOCC1)[C@H](CCC1(C(F)(F)F)CC1)CO2] | | | ND | + | | |
| 339 | 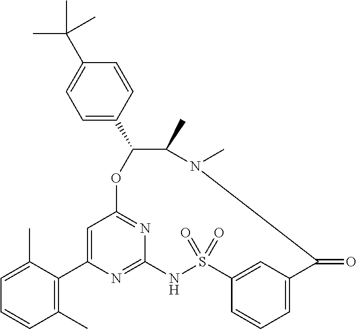 [SMILES: Cc1cccc(C)c1-c1cc2nc(n1)NS(=O)(=O)c1cccc(c1)C(=O)N(C)[C@H](C)[C@@H](c1ccc(C(C)(C)C)cc1)O2] | +++ | +++ | +++ | ++ | | |
| 1044 | 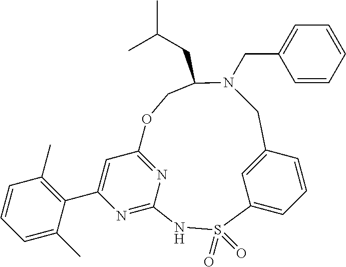 [SMILES: Cc1cccc(C)c1-c1cc2nc(n1)NS(=O)(=O)c1cccc(c1)CN(Cc1ccccc1)[C@H](CC(C)C)CO2] | | | +++ | ++ | | |
| 1043 | 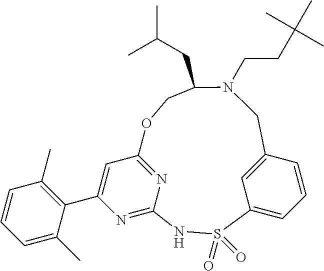 [SMILES: Cc1cccc(C)c1-c1cc2nc(n1)NS(=O)(=O)c1cccc(c1)CN(CCC(C)(C)C)[C@H](CC(C)C)CO2] | +++ | +++ | +++ | +++ | | |

4A Biological Activity Data

Table 7 represents CFTR modulating activity for representative compounds of the invention generated using one or more of the assays disclosed herein (EC₅₀: +++ is <1 M; ++ is 1-<3 μM; + is 3-<30.M; and ND is “not detected in this assay.” 00 Activity: +++ is >60%; ++ is 30-60%; + is <30%).

TABLE 7

| | | | 3T3 | Ent. | Ent. A | Ent. | Ent. B |
|---|---|---|---|---|---|---|---|
| | | 3T3 | Max | A | Max | B | Max |
| Cmpd | | EC₅₀ | Activity | EC₅₀ | Activity | EC₅₀ | Activity |
| Number | Structure | (μM) | (%) | (μM) | (%) | (μM) | (%) |
| 1042 | 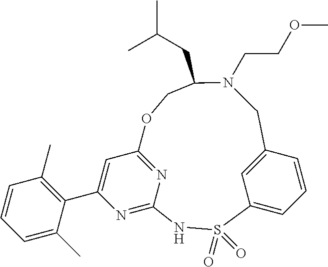 [SMILES: COCCN1Cc2cccc(c2)S(=O)(=O)Nc2nc(cc(-c3c(C)cccc3C)n2)OC[C@H]1CC(C)C] | | | +++ | + + | | |
| 1052 | 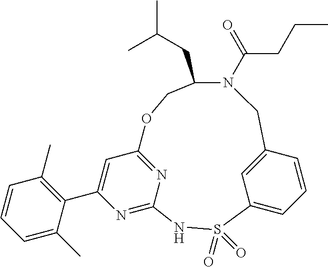 [SMILES: CCCC(=O)N1Cc2cccc(c2)S(=O)(=O)Nc2nc(cc(-c3c(C)cccc3C)n2)OC[C@H]1CC(C)C] | | | ND | ++ | | |
| 1051 | 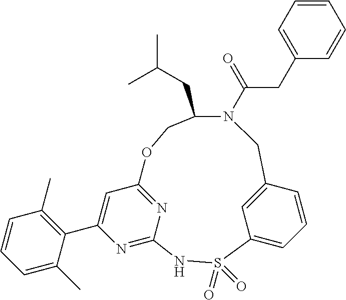 [SMILES: Cc1cccc(C)c1-c1cc2nc(n1)NS(=O)(=O)c1cccc(c1)CN(C(=O)Cc1ccccc1)[C@H](CC(C)C)CO2] | | | ++ | ++ | | |
| 1050 | 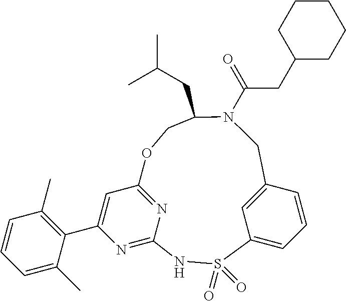 [SMILES: Cc1cccc(C)c1-c1cc2nc(n1)NS(=O)(=O)c1cccc(c1)CN(C(=O)CC1CCCCC1)[C@H](CC(C)C)CO2] | | | ++ | + | | |
| 1049 | 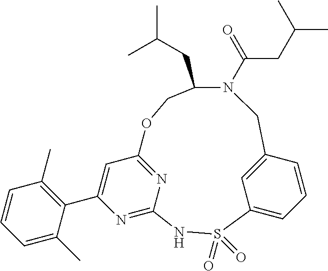 [SMILES: Cc1cccc(C)c1-c1cc2nc(n1)NS(=O)(=O)c1cccc(c1)CN(C(=O)CC(C)C)[C@H](CC(C)C)CO2] | | | ND | + | | |
| 1048 | 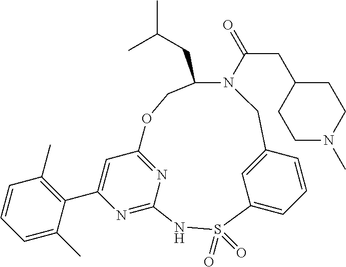 [SMILES: Cc1cccc(C)c1-c1cc2nc(n1)NS(=O)(=O)c1cccc(c1)CN(C(=O)CC1CCN(C)CC1)[C@H](CC(C)C)CO2] | | | ++ | +++ | | |
| 417 | 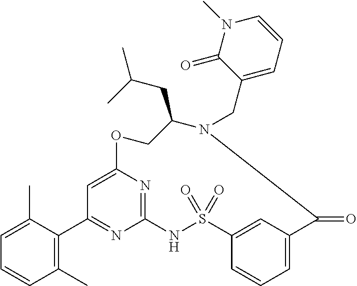 [SMILES: Cc1cccc(C)c1-c1cc2nc(n1)NS(=O)(=O)c1cccc(c1)C(=O)N(Cc1cccn(C)c1=O)[C@H](CC(C)C)CO2] | | | +++ | +++ | | |
| 83 | 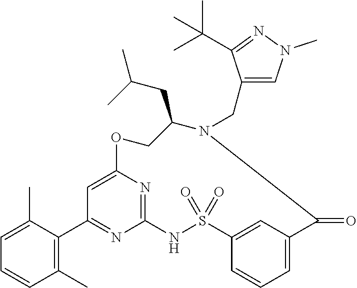 [SMILES: Cc1cccc(C)c1-c1cc2nc(n1)NS(=O)(=O)c1cccc(c1)C(=O)N(Cc1cn(C)nc1C(C)(C)C)[C@H](CC(C)C)CO2] | | | +++ | +++ | | |
| 416 | 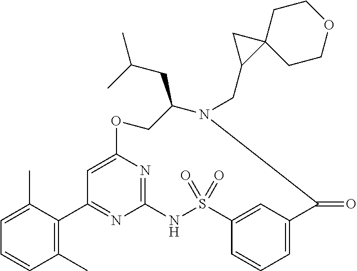 [SMILES: Cc1cccc(C)c1-c1cc2nc(n1)NS(=O)(=O)c1cccc(c1)C(=O)N(CC1CC13CCOCC3)[C@H](CC(C)C)CO2] | | | +++ | +++ | | |
| 988 | 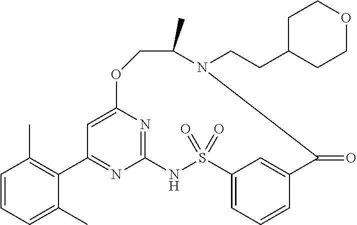 [SMILES: Cc1cccc(C)c1-c1cc2nc(n1)NS(=O)(=O)c1cccc(c1)C(=O)N(CCC1CCOCC1)[C@H](C)CO2] | | | + | + | | |
| 987 | 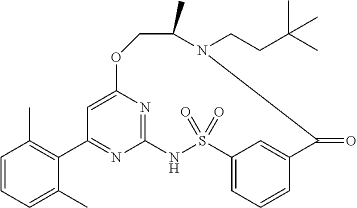 [SMILES: Cc1cccc(C)c1-c1cc2nc(n1)NS(=O)(=O)c1cccc(c1)C(=O)N(CCC(C)(C)C)[C@H](C)CO2] | | | +++ | + | | |
| 986 | 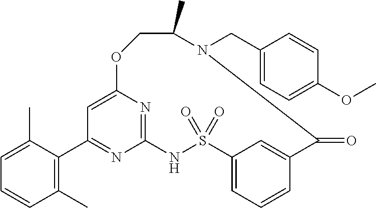 [SMILES: COc1ccc(CN2C(=O)c3cccc(c3)S(=O)(=O)Nc3nc(cc(-c4c(C)cccc4C)n3)OC[C@H]2C)cc1] | | | ++ | ++ | | |
| 1046 | 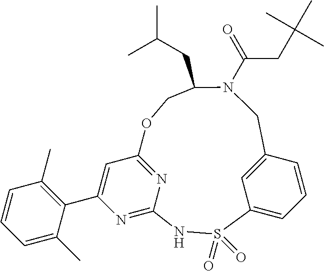 [SMILES: Cc1cccc(C)c1-c1cc2nc(n1)NS(=O)(=O)c1cccc(c1)CN(C(=O)CC(C)(C)C)[C@H](CC(C)C)CO2] | | | ND | + | | |
| 45 | 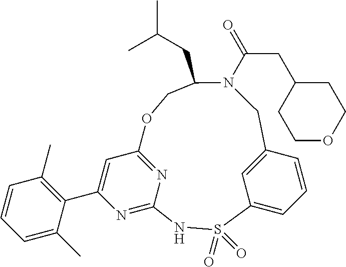 [SMILES: Cc1cccc(C)c1-c1cc2nc(n1)NS(=O)(=O)c1cccc(c1)CN(C(=O)CC1CCOCC1)[C@H](CC(C)C)CO2] | | | ND | + | | |
| 1025 | 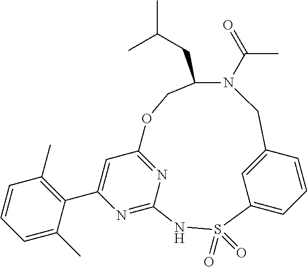 [SMILES: CC(=O)N1Cc2cccc(c2)S(=O)(=O)Nc2nc(cc(-c3c(C)cccc3C)n2)OC[C@H]1CC(C)C] | | | ND | + | | |
| 974 | 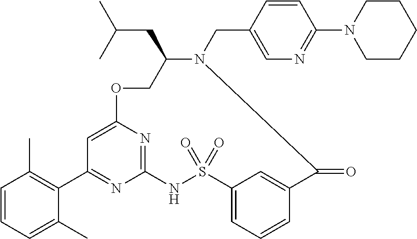 [SMILES: Cc1cccc(C)c1-c1cc2nc(n1)NS(=O)(=O)c1cccc(c1)C(=O)N(Cc1ccc(N3CCCCC3)nc1)[C@H](CC(C)C)CO2] | +++ | +++ | +++ | +++ | | |
| 973 | 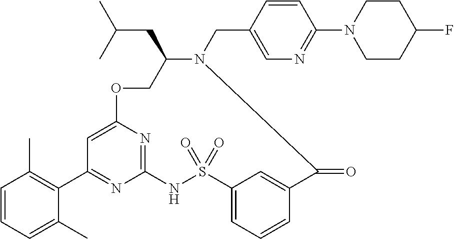 [SMILES: Cc1cccc(C)c1-c1cc2nc(n1)NS(=O)(=O)c1cccc(c1)C(=O)N(Cc1ccc(N3CCC(F)CC3)nc1)[C@H](CC(C)C)CO2] | | | +++ | +++ | | |
| 972 | 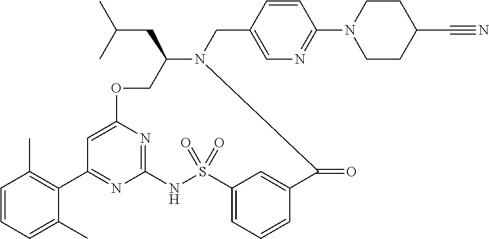 [SMILES: Cc1cccc(C)c1-c1cc2nc(n1)NS(=O)(=O)c1cccc(c1)C(=O)N(Cc1ccc(N3CCC(C#N)CC3)nc1)[C@H](CC(C)C)CO2] | | | +++ | +++ | | |
| 971 | 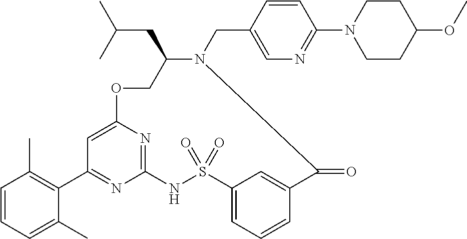 [SMILES: COC1CCN(c2ccc(CN3C(=O)c4cccc(c4)S(=O)(=O)Nc4nc(cc(-c5c(C)cccc5C)n4)OC[C@H]3CC(C)C)cn2)CC1] | | | +++ | +++ | | |
| 970 | 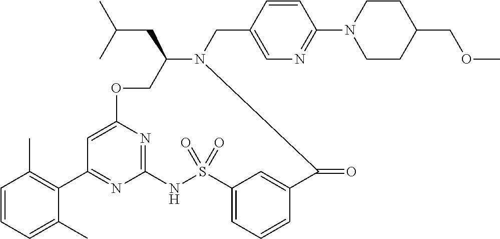 [SMILES: COCC1CCN(c2ccc(CN3C(=O)c4cccc(c4)S(=O)(=O)Nc4nc(cc(-c5c(C)cccc5C)n4)OC[C@H]3CC(C)C)cn2)CC1] | | | +++ | ++ | | |
| 969 | 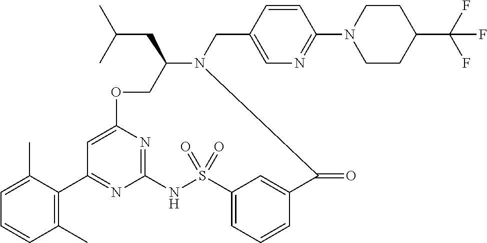 [SMILES: Cc1cccc(C)c1-c1cc2nc(n1)NS(=O)(=O)c1cccc(c1)C(=O)N(Cc1ccc(N3CCC(C(F)(F)F)CC3)nc1)[C@H](CC(C)C)CO2] | | | +++ | ++ | | |
| 337 | 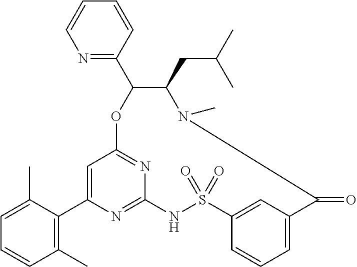 [SMILES: Cc1cccc(C)c1-c1cc2nc(n1)NS(=O)(=O)c1cccc(c1)C(=O)N(C)[C@H](CC(C)C)C(c1ccccn1)O2] | | | +++ | +++ | | |
| 11 | 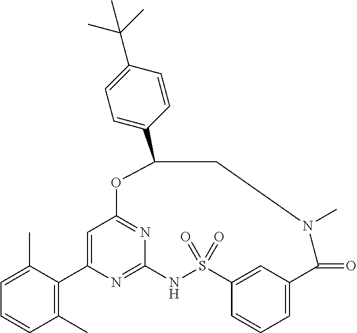 [SMILES: Cc1cccc(C)c1-c1cc2nc(n1)NS(=O)(=O)c1cccc(c1)C(=O)N(C)C[C@H](c1ccc(C(C)(C)C)cc1)O2] | +++ | +++ | +++ | +++ | | |
| 12 | 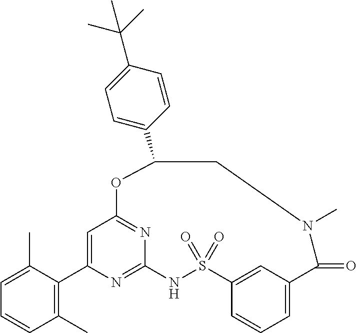 [SMILES: Cc1cccc(C)c1-c1cc2nc(n1)NS(=O)(=O)c1cccc(c1)C(=O)N(C)C[C@@H](c1ccc(C(C)(C)C)cc1)O2] | | | +++ | ++ | | |
| 16 | 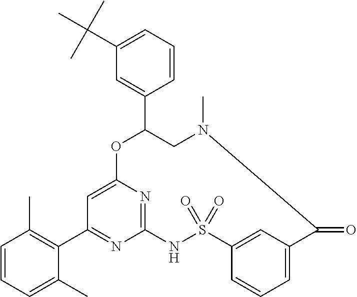 [SMILES: Cc1cccc(C)c1-c1cc2nc(n1)NS(=O)(=O)c1cccc(c1)C(=O)N(C)CC(c1cccc(C(C)(C)C)c1)O2] | | | +++ | ++ | | |
| 670 | 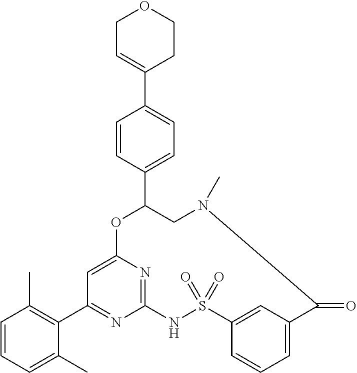 [SMILES: Cc1cccc(C)c1-c1cc2nc(n1)NS(=O)(=O)c1cccc(c1)C(=O)N(C)CC(c1ccc(C3=CCOCC3)cc1)O2] | | | ++ | ++ | | |
| 671 | 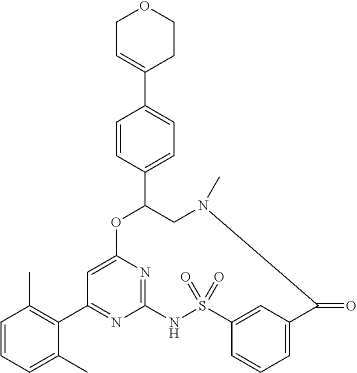 [SMILES: Cc1cccc(C)c1-c1cc2nc(n1)NS(=O)(=O)c1cccc(c1)C(=O)N(C)CC(c1ccc(C3=CCOCC3)cc1)O2] | | | +++ | +++ | | |
| 13 | 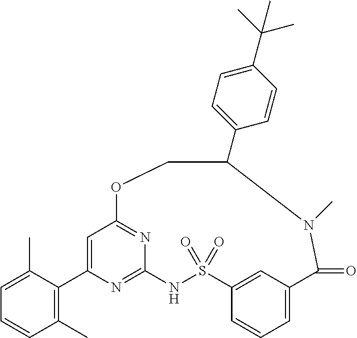 [SMILES: Cc1cccc(C)c1-c1cc2nc(n1)NS(=O)(=O)c1cccc(c1)C(=O)N(C)C(c1ccc(C(C)(C)C)cc1)CO2] | | | +++ | ++ | | |
| 15 | 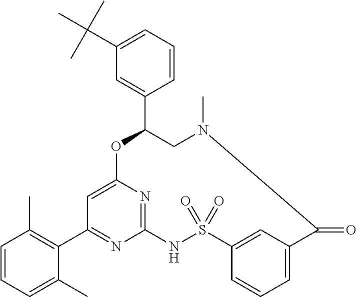 [SMILES: Cc1cccc(C)c1-c1cc2nc(n1)NS(=O)(=O)c1cccc(c1)C(=O)N(C)C[C@H](c1cccc(C(C)(C)C)c1)O2] | | | + | + | | |
| 17 | 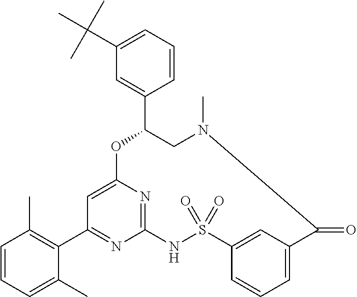 [SMILES: Cc1cccc(C)c1-c1cc2nc(n1)NS(=O)(=O)c1cccc(c1)C(=O)N(C)C[C@@H](c1cccc(C(C)(C)C)c1)O2] | | | +++ | +++ | | |
| 686 | 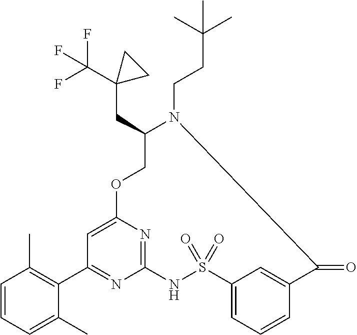 [SMILES: Cc1cccc(C)c1-c1cc2nc(n1)NS(=O)(=O)c1cccc(c1)C(=O)N(CCC(C)(C)C)[C@H](CC1(C(F)(F)F)CC1)CO2] | | | +++ | + | | |
| 687 | 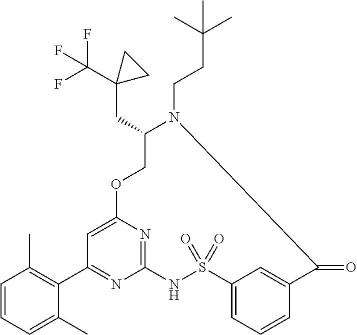 [SMILES: Cc1cccc(C)c1-c1cc2nc(n1)NS(=O)(=O)c1cccc(c1)C(=O)N(CCC(C)(C)C)[C@@H](CC1(C(F)(F)F)CC1)CO2] | | | | | | |
| 1047 | 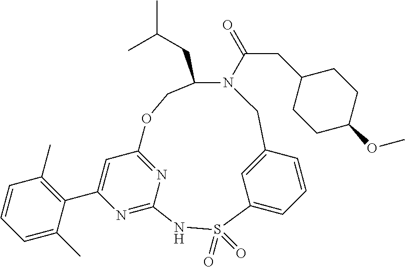 | | | ND | + | | |
| 1045 | 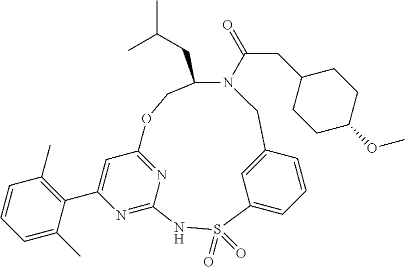 | | | ND | + | | |
| 1041 | 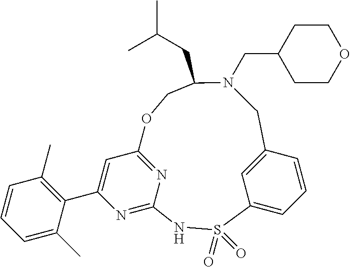 [SMILES: Cc1cccc(C)c1-c1cc2nc(n1)NS(=O)(=O)c1cccc(c1)CN(CC1CCOCC1)[C@H](CC(C)C)CO2] | | | +++ | +++ | | |
| 1040 | 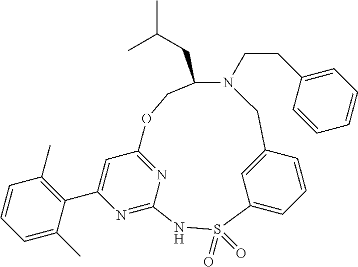 [SMILES: Cc1cccc(C)c1-c1cc2nc(n1)NS(=O)(=O)c1cccc(c1)CN(CCc1ccccc1)[C@H](CC(C)C)CO2] | | | +++ | ++ | | |
| 1039 | 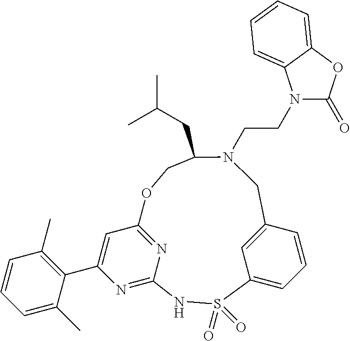 [SMILES: Cc1cccc(C)c1-c1cc2nc(n1)NS(=O)(=O)c1cccc(c1)CN(CCn1c(=O)oc3ccccc31)[C@H](CC(C)C)CO2] | | | +++ | ++ | | |
| 1038 | 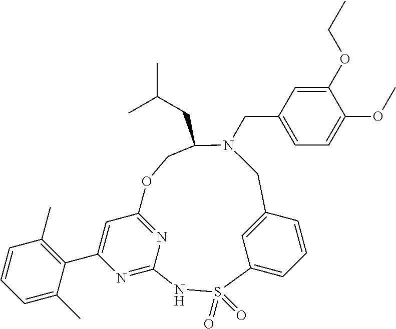 [SMILES: CCOc1cc(CN2Cc3cccc(c3)S(=O)(=O)Nc3nc(cc(-c4c(C)cccc4C)n3)OC[C@H]2CC(C)C)ccc1OC] | | | +++ | ++ | | |
| 1037 | 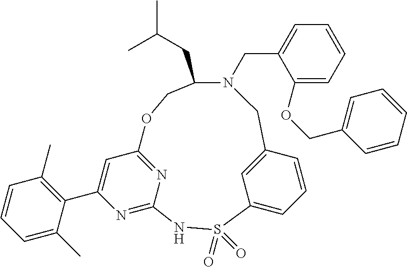 [SMILES: Cc1cccc(C)c1-c1cc2nc(n1)NS(=O)(=O)c1cccc(c1)CN(Cc1ccccc1OCc1ccccc1)[C@H](CC(C)C)CO2] | | | +++ | ++ | | |
| 1036 | 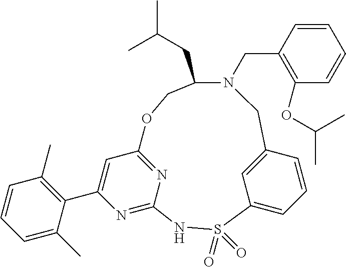 [SMILES: Cc1cccc(C)c1-c1cc2nc(n1)NS(=O)(=O)c1cccc(c1)CN(Cc1ccccc1OC(C)C)[C@H](CC(C)C)CO2] | | | +++ | ++ | | |
| 1035 | 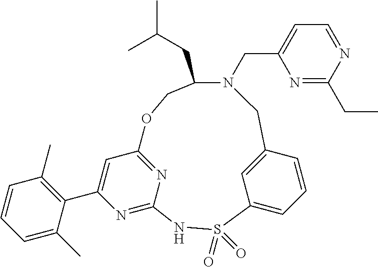 [SMILES: CCc1nccc(CN2Cc3cccc(c3)S(=O)(=O)Nc3nc(cc(-c4c(C)cccc4C)n3)OC[C@H]2CC(C)C)n1] | | | +++ | ++ | | |
| 1034 | 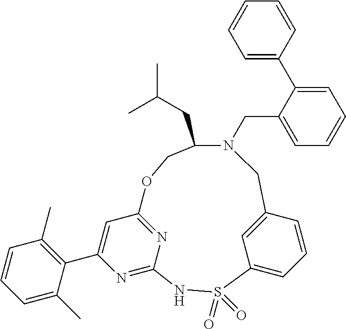 [SMILES: Cc1cccc(C)c1-c1cc2nc(n1)NS(=O)(=O)c1cccc(c1)CN(Cc1ccccc1-c1ccccc1)[C@H](CC(C)C)CO2] | | | +++ | ++ | | |
| 1033 | 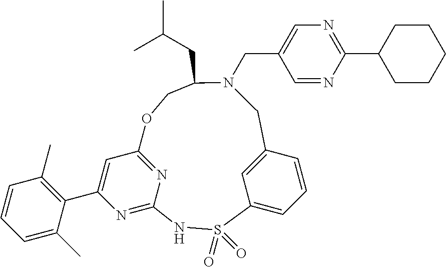 [SMILES: Cc1cccc(C)c1-c1cc2nc(n1)NS(=O)(=O)c1cccc(c1)CN(Cc1cnc(C3CCCCC3)nc1)[C@H](CC(C)C)CO2] | | | +++ | ++ | | |
| 1032 | 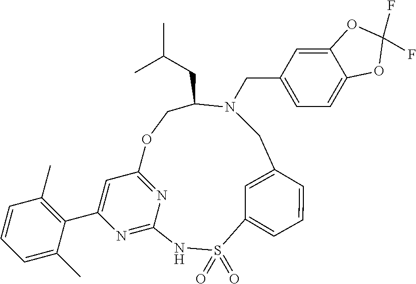 [SMILES: Cc1cccc(C)c1-c1cc2nc(n1)NS(=O)(=O)c1cccc(c1)CN(Cc1ccc3c(c1)OC(F)(F)O3)[C@H](CC(C)C)CO2] | | | +++ | ++ | | |
| 1031 | 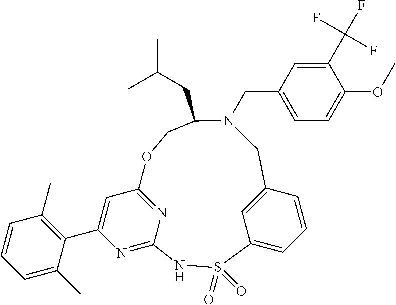 [SMILES: COc1ccc(CN2Cc3cccc(c3)S(=O)(=O)Nc3nc(cc(-c4c(C)cccc4C)n3)OC[C@H]2CC(C)C)cc1C(F)(F)F] | | | +++ | ++ | | |
| 1030 | 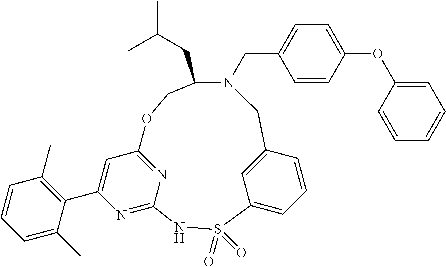 [SMILES: Cc1cccc(C)c1-c1cc2nc(n1)NS(=O)(=O)c1cccc(c1)CN(Cc1ccc(Oc3ccccc3)cc1)[C@H](CC(C)C)CO2] | | | +++ | ++ | | |
| 1029 | 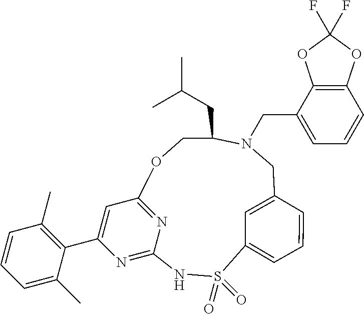 [SMILES: Cc1cccc(C)c1-c1cc2nc(n1)NS(=O)(=O)c1cccc(c1)CN(Cc1cccc3c1OC(F)(F)O3)[C@H](CC(C)C)CO2] | +++ | +++ | +++ | ++ | | |
| 1028 | 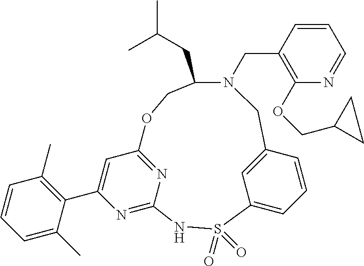 [SMILES: Cc1cccc(C)c1-c1cc2nc(n1)NS(=O)(=O)c1cccc(c1)CN(Cc1cccnc1OCC1CC1)[C@H](CC(C)C)CO2] | | | +++ | +++ | | |
| 47 | 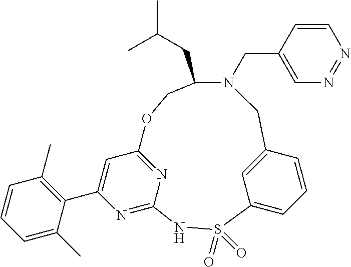 [SMILES: Cc1cccc(C)c1-c1cc2nc(n1)NS(=O)(=O)c1cccc(c1)CN(Cc1ccnnc1)[C@H](CC(C)C)CO2] | +++ | +++ | +++ | +++ | | |
| 1026 | 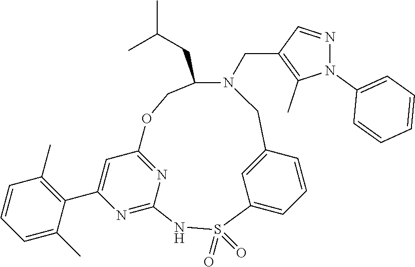 [SMILES: Cc1cccc(C)c1-c1cc2nc(n1)NS(=O)(=O)c1cccc(c1)CN(Cc1cnn(-c3ccccc3)c1C)[C@H](CC(C)C)CO2] | +++ | +++ | +++ | +++ | | |
| 1027 | 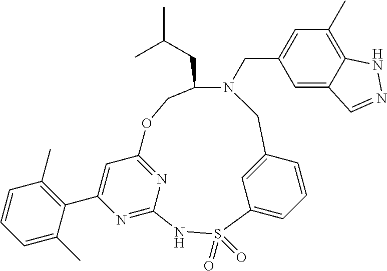 [SMILES: Cc1cccc(C)c1-c1cc2nc(n1)NS(=O)(=O)c1cccc(c1)CN(Cc1cc(C)c3[nH]ncc3c1)[C@H](CC(C)C)CO2] | | | +++ | +++ | | |
| 1024 | 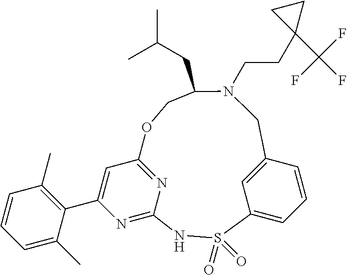 [SMILES: Cc1cccc(C)c1-c1cc2nc(n1)NS(=O)(=O)c1cccc(c1)CN(CCC1(C(F)(F)F)CC1)[C@H](CC(C)C)CO2] | +++ | +++ | +++ | ++ | | |
| 1023 | 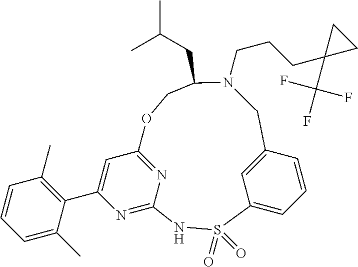 [SMILES: Cc1cccc(C)c1-c1cc2nc(n1)NS(=O)(=O)c1cccc(c1)CN(CCCC1(C(F)(F)F)CC1)[C@H](CC(C)C)CO2] | +++ | +++ | +++ | ++ | | |
| 1021 | 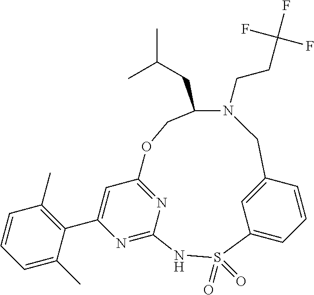 [SMILES: Cc1cccc(C)c1-c1cc2nc(n1)NS(=O)(=O)c1cccc(c1)CN(CCC(F)(F)F)[C@H](CC(C)C)CO2] | +++ | +++ | +++ | ++ | | |
| 373 | 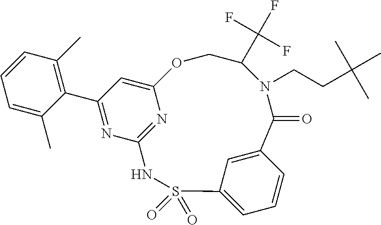 [SMILES: Cc1cccc(C)c1-c1cc2nc(n1)NS(=O)(=O)c1cccc(c1)C(=O)N(CCC(C)(C)C)C(C(F)(F)F)CO2] | ++ | +++ | | | | |
| 668 | 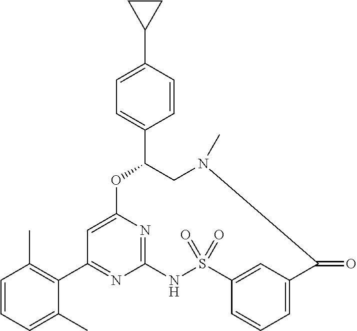 [SMILES: Cc1cccc(C)c1-c1cc2nc(n1)NS(=O)(=O)c1cccc(c1)C(=O)N(C)C[C@@H](c1ccc(C3CC3)cc1)O2] | +++ | +++ | | | | |
| 669 | 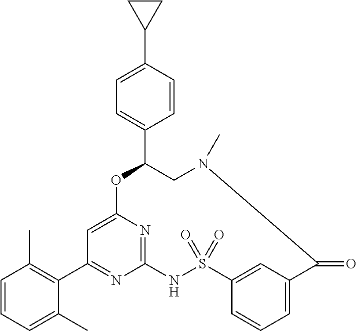 [SMILES: Cc1cccc(C)c1-c1cc2nc(n1)NS(=O)(=O)c1cccc(c1)C(=O)N(C)C[C@H](c1ccc(C3CC3)cc1)O2] | +++ | +++ | +++ | ++ | | |
| 102 | 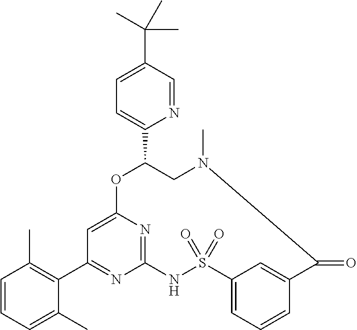 [SMILES: Cc1cccc(C)c1-c1cc2nc(n1)NS(=O)(=O)c1cccc(c1)C(=O)N(C)C[C@@H](c1ccc(C(C)(C)C)cn1)O2] | +++ | +++ | ++ | + | | |
| 103 | 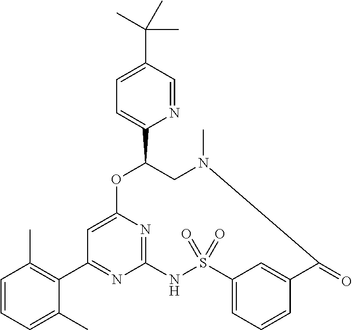 [SMILES: Cc1cccc(C)c1-c1cc2nc(n1)NS(=O)(=O)c1cccc(c1)C(=O)N(C)C[C@H](c1ccc(C(C)(C)C)cn1)O2] | +++ | +++ | +++ | +++ | | |
| 85 | 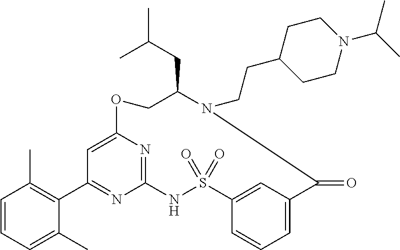 [SMILES: Cc1cccc(C)c1-c1cc2nc(n1)NS(=O)(=O)c1cccc(c1)C(=O)N(CCC1CCN(C(C)C)CC1)[C@H](CC(C)C)CO2] | +++ | +++ | +++ | +++ | | |
| 1107 | 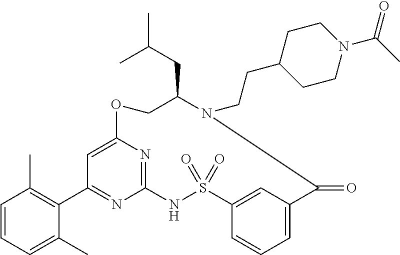 [SMILES: CC(=O)N1CCC(CCN2C(=O)c3cccc(c3)S(=O)(=O)Nc3nc(cc(-c4c(C)cccc4C)n3)OC[C@H]2CC(C)C)CC1] | +++ | +++ | +++ | +++ | | |
| 509 | 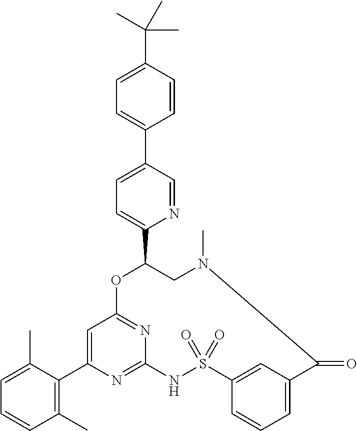 [SMILES: Cc1cccc(C)c1-c1cc2nc(n1)NS(=O)(=O)c1cccc(c1)C(=O)N(C)C[C@H](c1ccc(-c3ccc(C(C)(C)C)cc3)cn1)O2] | | | ND | + | | |
| 510 | 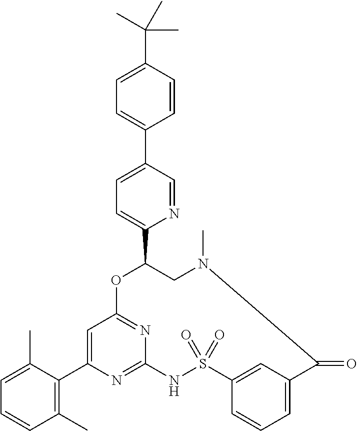 [SMILES: Cc1cccc(C)c1-c1cc2nc(n1)NS(=O)(=O)c1cccc(c1)C(=O)N(C)C[C@H](c1ccc(-c3ccc(C(C)(C)C)cc3)cn1)O2] | | | +++ | +++ | | |
| 89 | 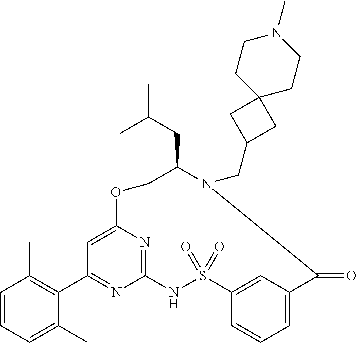 [SMILES: Cc1cccc(C)c1-c1cc2nc(n1)NS(=O)(=O)c1cccc(c1)C(=O)N(CC1CC3(CCN(C)CC3)C1)[C@H](CC(C)C)CO2] | | | +++ | +++ | | |
| 682 | 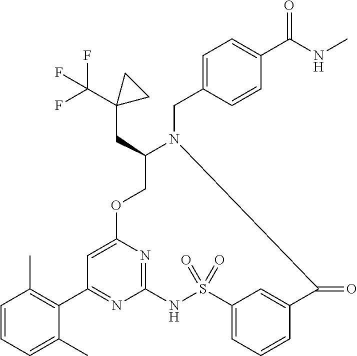 [SMILES: CNC(=O)c1ccc(CN2C(=O)c3cccc(c3)S(=O)(=O)Nc3nc(cc(-c4c(C)cccc4C)n3)OC[C@H]2CC2(C(F)(F)F)CC2)cc1] | | | ++ | + | | |
| 683 | 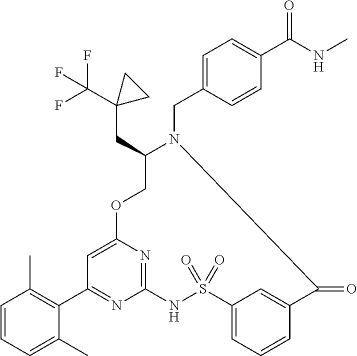 [SMILES: CNC(=O)c1ccc(CN2C(=O)c3cccc(c3)S(=O)(=O)Nc3nc(cc(-c4c(C)cccc4C)n3)OC[C@H]2CC2(C(F)(F)F)CC2)cc1] | | | +++ | +++ | | |
| 415 | 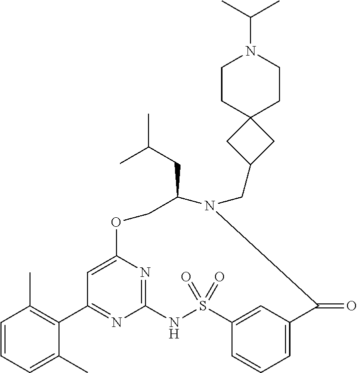 [SMILES: Cc1cccc(C)c1-c1cc2nc(n1)NS(=O)(=O)c1cccc(c1)C(=O)N(CC1CC3(CCN(C(C)C)CC3)C1)[C@H](CC(C)C)CO2] | | | +++ | +++ | | |
| 90 | 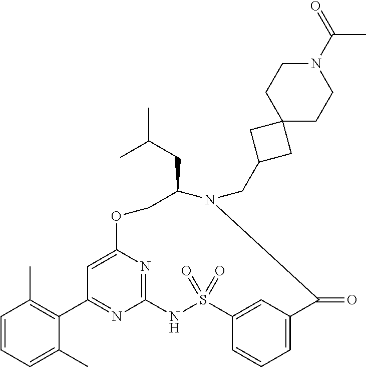 [SMILES: CC(=O)N1CCC2(CC1)CC(CN1C(=O)c3cccc(c3)S(=O)(=O)Nc3nc(cc(-c4c(C)cccc4C)n3)OC[C@H]1CC(C)C)C2] | | | +++ | +++ | | |
| 968 | 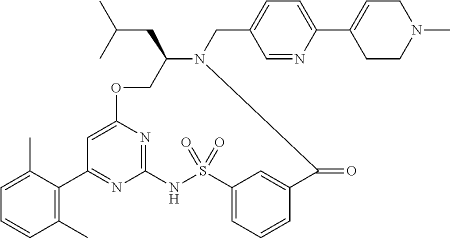 [SMILES: Cc1cccc(C)c1-c1cc2nc(n1)NS(=O)(=O)c1cccc(c1)C(=O)N(Cc1ccc(C3=CCN(C)CC3)nc1)[C@H](CC(C)C)CO2] | | | | | | |
| 967 | 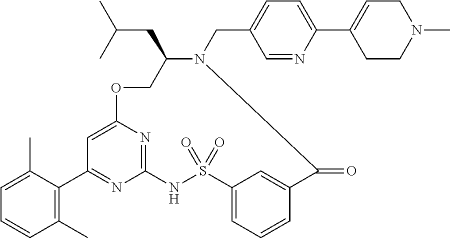 [SMILES: Cc1cccc(C)c1-c1cc2nc(n1)NS(=O)(=O)c1cccc(c1)C(=O)N(Cc1ccc(C3=CCN(C)CC3)nc1)[C@H](CC(C)C)CO2] | | | +++ | | | |
| 680 | 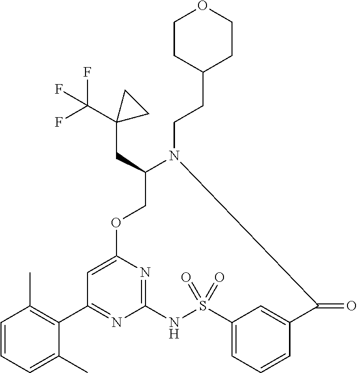 [SMILES: Cc1cccc(C)c1-c1cc2nc(n1)NS(=O)(=O)c1cccc(c1)C(=O)N(CCC1CCOCC1)[C@H](CC1(C(F)(F)F)CC1)CO2] | | | +++ | + | | |
| 681 | 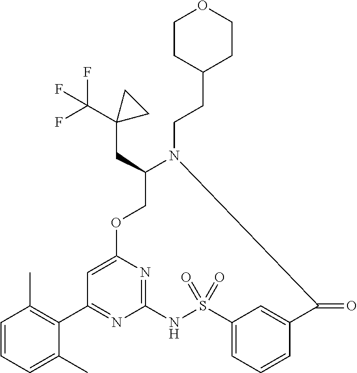 [SMILES: Cc1cccc(C)c1-c1cc2nc(n1)NS(=O)(=O)c1cccc(c1)C(=O)N(CCC1CCOCC1)[C@H](CC1(C(F)(F)F)CC1)CO2] | | | +++ | +++ | | |
| 678 | 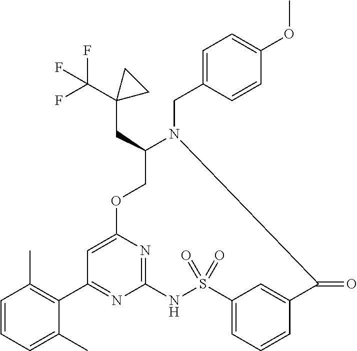 [SMILES: COc1ccc(CN2C(=O)c3cccc(c3)S(=O)(=O)Nc3nc(cc(-c4c(C)cccc4C)n3)OC[C@H]2CC2(C(F)(F)F)CC2)cc1] | | | +++ | ++ | | |
| 679 | 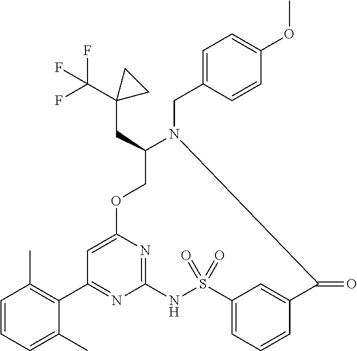 [SMILES: COc1ccc(CN2C(=O)c3cccc(c3)S(=O)(=O)Nc3nc(cc(-c4c(C)cccc4C)n3)OC[C@H]2CC2(C(F)(F)F)CC2)cc1] | | | +++ | +++ | | |
| 684 | 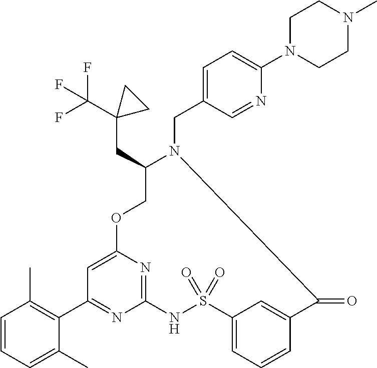 [SMILES: Cc1cccc(C)c1-c1cc2nc(n1)NS(=O)(=O)c1cccc(c1)C(=O)N(Cc1ccc(N3CCN(C)CC3)nc1)[C@H](CC1(C(F)(F)F)CC1)CO2] | | | +++ | +++ | | |
| 685 | 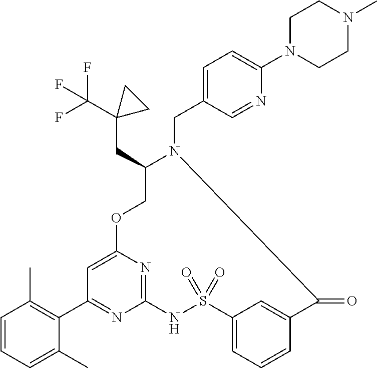 [SMILES: Cc1cccc(C)c1-c1cc2nc(n1)NS(=O)(=O)c1cccc(c1)C(=O)N(Cc1ccc(N3CCN(C)CC3)nc1)[C@H](CC1(C(F)(F)F)CC1)CO2] | | | +++ | +++ | | |
| 1022 | 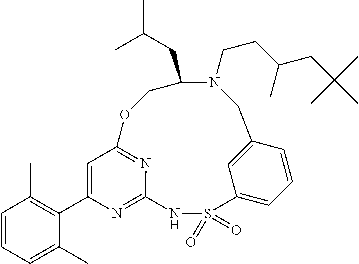 [SMILES: Cc1cccc(C)c1-c1cc2nc(n1)NS(=O)(=O)c1cccc(c1)CN(CCC(C)CC(C)(C)C)[C@H](CC(C)C)CO2] | | | +++ | ++ | | |
| 984 | 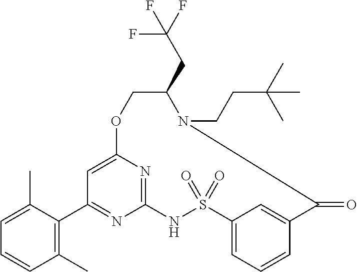 [SMILES: Cc1cccc(C)c1-c1cc2nc(n1)NS(=O)(=O)c1cccc(c1)C(=O)N(CCC(C)(C)C)[C@H](CC(F)(F)F)CO2] | | | ++ | ++ | | |
| 985 | 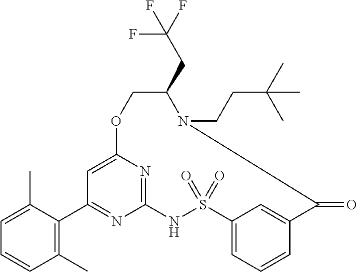 [SMILES: Cc1cccc(C)c1-c1cc2nc(n1)NS(=O)(=O)c1cccc(c1)C(=O)N(CCC(C)(C)C)[C@H](CC(F)(F)F)CO2] | | | +++ | ++ | | |
| 966 | 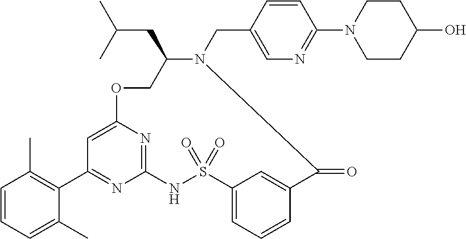 [SMILES: Cc1cccc(C)c1-c1cc2nc(n1)NS(=O)(=O)c1cccc(c1)C(=O)N(Cc1ccc(N3CCC(O)CC3)nc1)[C@H](CC(C)C)CO2] | | | +++ | +++ | | |
| 956 | 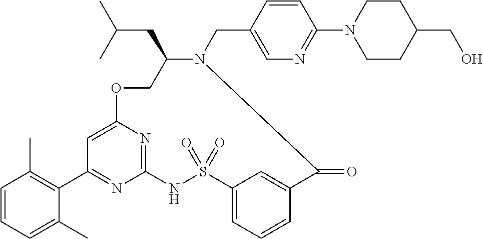 [SMILES: Cc1cccc(C)c1-c1cc2nc(n1)NS(=O)(=O)c1cccc(c1)C(=O)N(Cc1ccc(N3CCC(CO)CC3)nc1)[C@H](CC(C)C)CO2] | | | +++ | +++ | | |
| 945 | 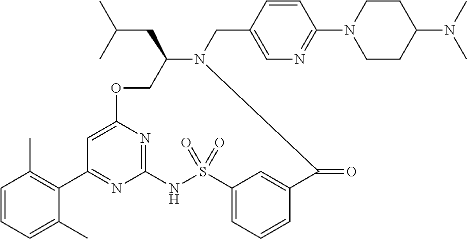 [SMILES: Cc1cccc(C)c1-c1cc2nc(n1)NS(=O)(=O)c1cccc(c1)C(=O)N(Cc1ccc(N3CCC(N(C)C)CC3)nc1)[C@H](CC(C)C)CO2] | | | +++ | +++ | | |
| 941 | 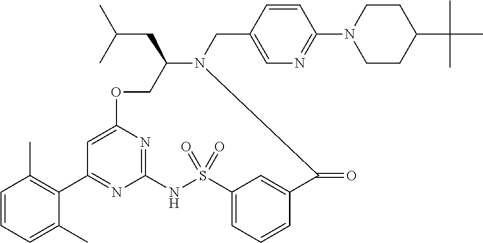 [SMILES: Cc1cccc(C)c1-c1cc2nc(n1)NS(=O)(=O)c1cccc(c1)C(=O)N(Cc1ccc(N3CCC(C(C)(C)C)CC3)nc1)[C@H](CC(C)C)CO2] | | | +++ | +++ | | |
| 940 | 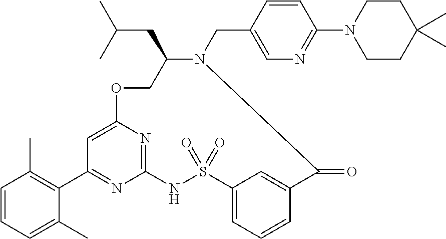 [SMILES: Cc1cccc(C)c1-c1cc2nc(n1)NS(=O)(=O)c1cccc(c1)C(=O)N(Cc1ccc(N3CCC(C)(C)CC3)nc1)[C@H](CC(C)C)CO2] | | | +++ | +++ | | |
| 939 | 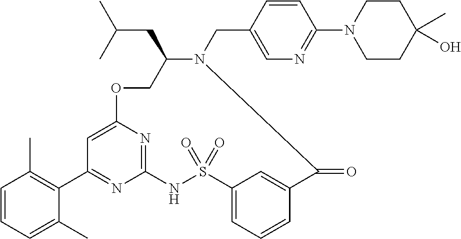 [SMILES: Cc1cccc(C)c1-c1cc2nc(n1)NS(=O)(=O)c1cccc(c1)C(=O)N(Cc1ccc(N3CCC(C)(O)CC3)nc1)[C@H](CC(C)C)CO2] | | | +++ | +++ | | |
| 938 | 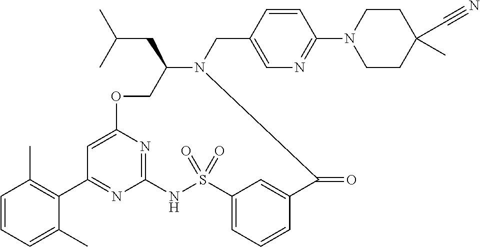 [SMILES: Cc1cccc(C)c1-c1cc2nc(n1)NS(=O)(=O)c1cccc(c1)C(=O)N(Cc1ccc(N3CCC(C)(C#N)CC3)nc1)[C@H](CC(C)C)CO2] | | | +++ | +++ | | |
| 937 | 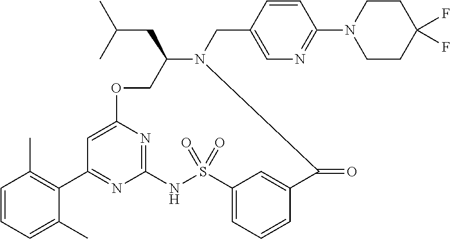 [SMILES: Cc1cccc(C)c1-c1cc2nc(n1)NS(=O)(=O)c1cccc(c1)C(=O)N(Cc1ccc(N3CCC(F)(F)CC3)nc1)[C@H](CC(C)C)CO2] | | | +++ | +++ | | |
| 936 | 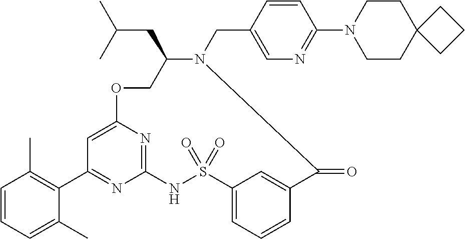 [SMILES: Cc1cccc(C)c1-c1cc2nc(n1)NS(=O)(=O)c1cccc(c1)C(=O)N(Cc1ccc(N3CCC4(CCC4)CC3)nc1)[C@H](CC(C)C)CO2] | | | +++ | +++ | | |
| 965 | 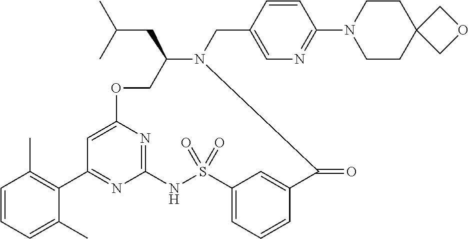 [SMILES: Cc1cccc(C)c1-c1cc2nc(n1)NS(=O)(=O)c1cccc(c1)C(=O)N(Cc1ccc(N3CCC4(CC3)COC4)nc1)[C@H](CC(C)C)CO2] | | | +++ | +++ | | |
| 964 | 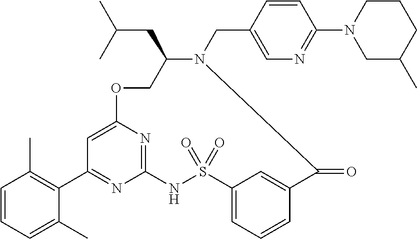 [SMILES: Cc1cccc(C)c1-c1cc2nc(n1)NS(=O)(=O)c1cccc(c1)C(=O)N(Cc1ccc(N3CCCC(C)C3)nc1)[C@H](CC(C)C)CO2] | | | +++ | +++ | | |
| 963 | 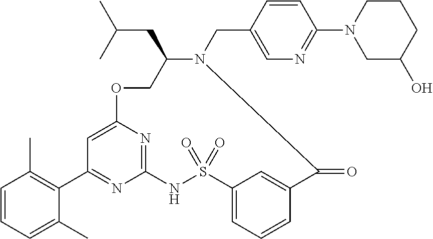 [SMILES: Cc1cccc(C)c1-c1cc2nc(n1)NS(=O)(=O)c1cccc(c1)C(=O)N(Cc1ccc(N3CCCC(O)C3)nc1)[C@H](CC(C)C)CO2] | | | +++ | +++ | | |
| 961 | 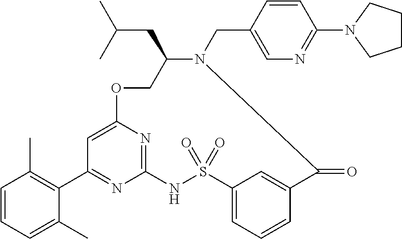 [SMILES: Cc1cccc(C)c1-c1cc2nc(n1)NS(=O)(=O)c1cccc(c1)C(=O)N(Cc1ccc(N3CCCC3)nc1)[C@H](CC(C)C)CO2] | | | +++ | +++ | | |
| 960 | 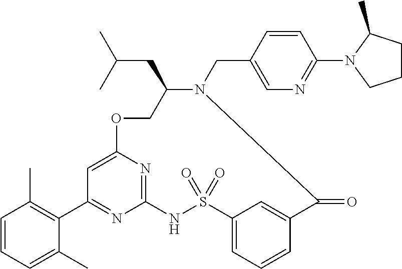 [SMILES: Cc1cccc(C)c1-c1cc2nc(n1)NS(=O)(=O)c1cccc(c1)C(=O)N(Cc1ccc(N3CCC[C@@H]3C)nc1)[C@H](CC(C)C)CO2] | | | +++ | +++ | | |
| 958 | 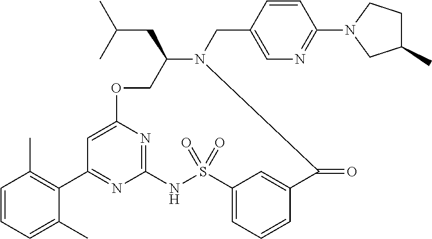 [SMILES: Cc1cccc(C)c1-c1cc2nc(n1)NS(=O)(=O)c1cccc(c1)C(=O)N(Cc1ccc(N3CC[C@@H](C)C3)nc1)[C@H](CC(C)C)CO2] | | | +++ | +++ | | |
| 953 | 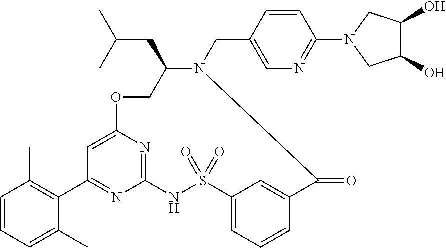 [SMILES: Cc1cccc(C)c1-c1cc2nc(n1)NS(=O)(=O)c1cccc(c1)C(=O)N(Cc1ccc(N3C[C@@H](O)[C@@H](O)C3)nc1)[C@H](CC(C)C)CO2] | | | +++ | +++ | | |
| 950 | 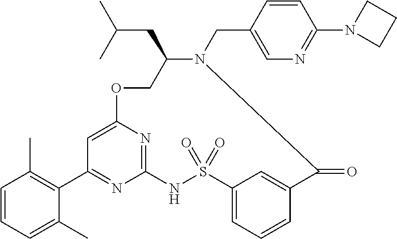 [SMILES: Cc1cccc(C)c1-c1cc2nc(n1)NS(=O)(=O)c1cccc(c1)C(=O)N(Cc1ccc(N3CCC3)nc1)[C@H](CC(C)C)CO2] | | | +++ | +++ | | |
| 949 | 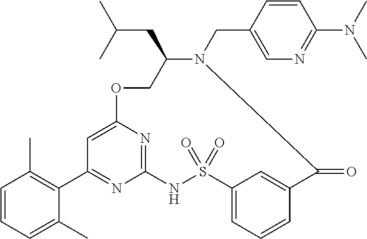 [SMILES: Cc1cccc(C)c1-c1cc2nc(n1)NS(=O)(=O)c1cccc(c1)C(=O)N(Cc1ccc(N(C)C)nc1)[C@H](CC(C)C)CO2] | | | +++ | +++ | | |
| 948 | 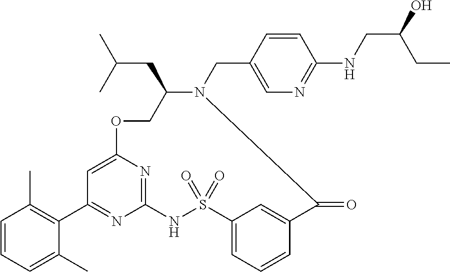 [SMILES: CC[C@H](O)CNc1ccc(CN2C(=O)c3cccc(c3)S(=O)(=O)Nc3nc(cc(-c4c(C)cccc4C)n3)OC[C@H]2CC(C)C)cn1] | | | ++ | +++ | | |
| 946 | 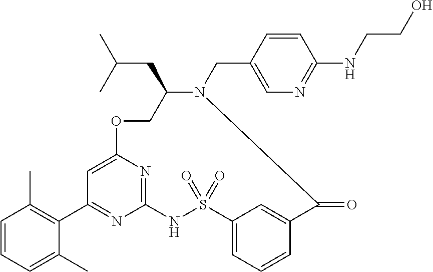 [SMILES: Cc1cccc(C)c1-c1cc2nc(n1)NS(=O)(=O)c1cccc(c1)C(=O)N(Cc1ccc(NCCO)nc1)[C@H](CC(C)C)CO2] | | | +++ | +++ | | |
| 944 | 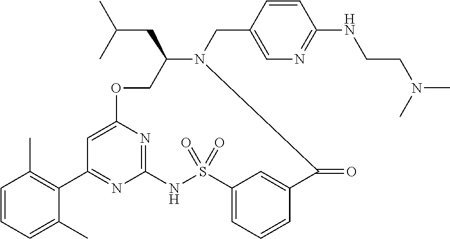 [SMILES: Cc1cccc(C)c1-c1cc2nc(n1)NS(=O)(=O)c1cccc(c1)C(=O)N(Cc1ccc(NCCN(C)C)nc1)[C@H](CC(C)C)CO2] | | | +++ | +++ | | |
| 943 | 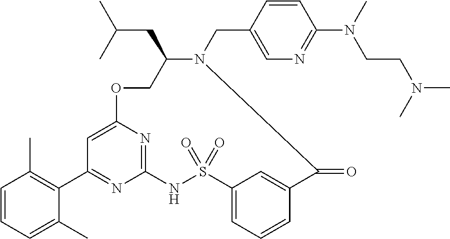 [SMILES: Cc1cccc(C)c1-c1cc2nc(n1)NS(=O)(=O)c1cccc(c1)C(=O)N(Cc1ccc(N(C)CCN(C)C)nc1)[C@H](CC(C)C)CO2] | | | +++ | +++ | | |
| 942 | 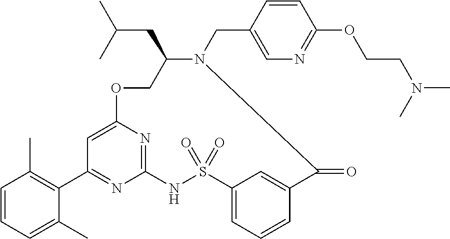 [SMILES: Cc1cccc(C)c1-c1cc2nc(n1)NS(=O)(=O)c1cccc(c1)C(=O)N(Cc1ccc(OCCN(C)C)nc1)[C@H](CC(C)C)CO2] | | | +++ | +++ | | |
| 962 | 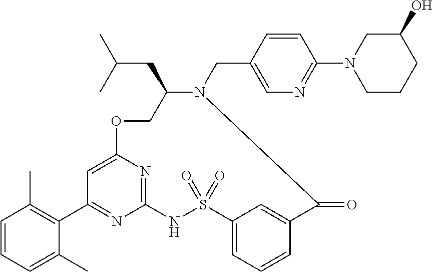 [SMILES: Cc1cccc(C)c1-c1cc2nc(n1)NS(=O)(=O)c1cccc(c1)C(=O)N(Cc1ccc(N3CCC[C@H](O)C3)nc1)[C@H](CC(C)C)CO2] | | | +++ | +++ | | |
| 959 | 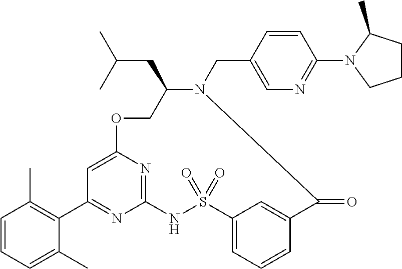 [SMILES: Cc1cccc(C)c1-c1cc2nc(n1)NS(=O)(=O)c1cccc(c1)C(=O)N(Cc1ccc(N3CCC[C@@H]3C)nc1)[C@H](CC(C)C)CO2] | | | +++ | +++ | | |
| 957 | 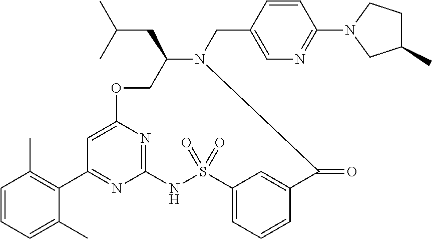 [SMILES: Cc1cccc(C)c1-c1cc2nc(n1)NS(=O)(=O)c1cccc(c1)C(=O)N(Cc1ccc(N3CC[C@@H](C)C3)nc1)[C@H](CC(C)C)CO2] | | | +++ | +++ | | |
| 952 | 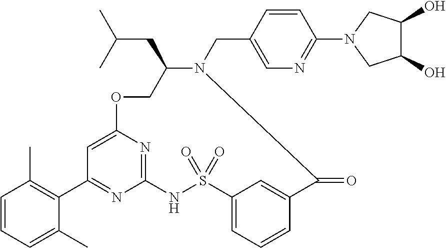 [SMILES: Cc1cccc(C)c1-c1cc2nc(n1)NS(=O)(=O)c1cccc(c1)C(=O)N(Cc1ccc(N3C[C@@H](O)[C@@H](O)C3)nc1)[C@H](CC(C)C)CO2] | | | +++ | +++ | | |
| 951 | 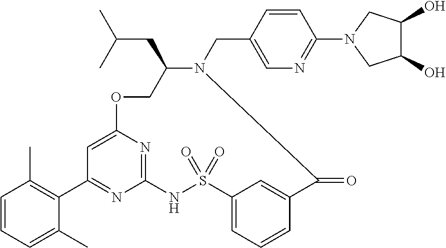 [SMILES: Cc1cccc(C)c1-c1cc2nc(n1)NS(=O)(=O)c1cccc(c1)C(=O)N(Cc1ccc(N3C[C@@H](O)[C@@H](O)C3)nc1)[C@H](CC(C)C)CO2] | | | +++ | +++ | | |
| 947 | 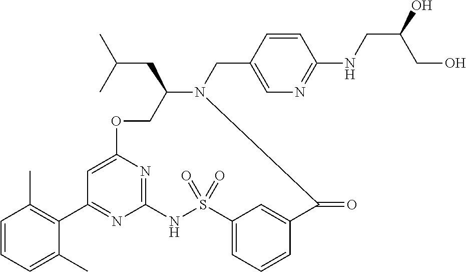 [SMILES: Cc1cccc(C)c1-c1cc2nc(n1)NS(=O)(=O)c1cccc(c1)C(=O)N(Cc1ccc(NC[C@@H](O)CO)nc1)[C@H](CC(C)C)CO2] | | | +++ | +++ | | |
| 955 | 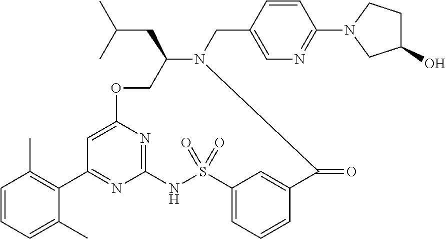 [SMILES: Cc1cccc(C)c1-c1cc2nc(n1)NS(=O)(=O)c1cccc(c1)C(=O)N(Cc1ccc(N3CC[C@@H](O)C3)nc1)[C@H](CC(C)C)CO2] | | | +++ | +++ | | |
| 954 | 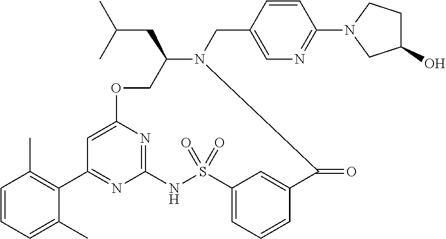 [SMILES: Cc1cccc(C)c1-c1cc2nc(n1)NS(=O)(=O)c1cccc(c1)C(=O)N(Cc1ccc(N3CC[C@@H](O)C3)nc1)[C@H](CC(C)C)CO2] | | | +++ | +++ | | |
| 935 | 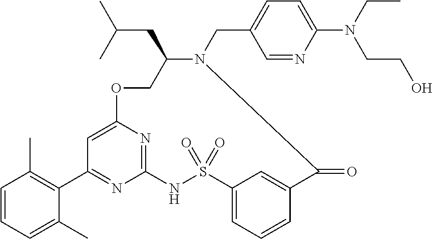 [SMILES: CCN(CCO)c1ccc(CN2C(=O)c3cccc(c3)S(=O)(=O)Nc3nc(cc(-c4c(C)cccc4C)n3)OC[C@H]2CC(C)C)cn1] | | | +++ | +++ | | |
| 95 | 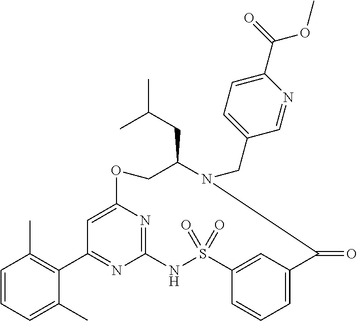 [SMILES: COC(=O)c1ccc(CN2C(=O)c3cccc(c3)S(=O)(=O)Nc3nc(cc(-c4c(C)cccc4C)n3)OC[C@H]2CC(C)C)cn1] | | | +++ | +++ | | |
| 93 | 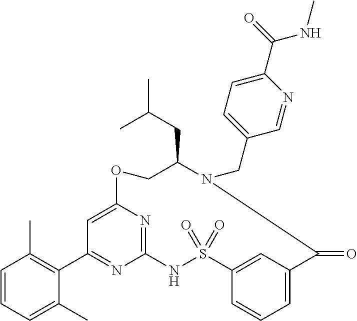 [SMILES: CNC(=O)c1ccc(CN2C(=O)c3cccc(c3)S(=O)(=O)Nc3nc(cc(-c4c(C)cccc4C)n3)OC[C@H]2CC(C)C)cn1] | | | +++ | +++ | | |
| 981 | 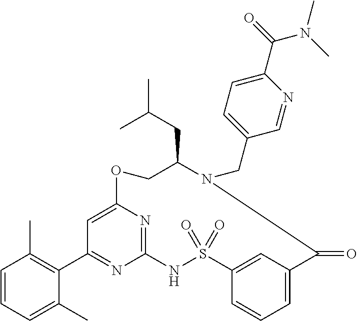 [SMILES: Cc1cccc(C)c1-c1cc2nc(n1)NS(=O)(=O)c1cccc(c1)C(=O)N(Cc1ccc(C(=O)N(C)C)nc1)[C@H](CC(C)C)CO2] | | | +++ | +++ | | |
| 414 | 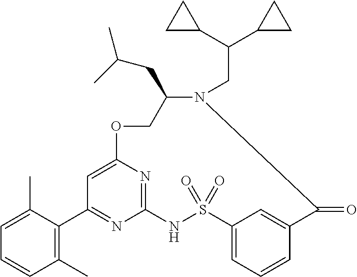 [SMILES: Cc1cccc(C)c1-c1cc2nc(n1)NS(=O)(=O)c1cccc(c1)C(=O)N(CC(C1CC1)C1CC1)[C@H](CC(C)C)CO2] | | | | | | |
| 413 | 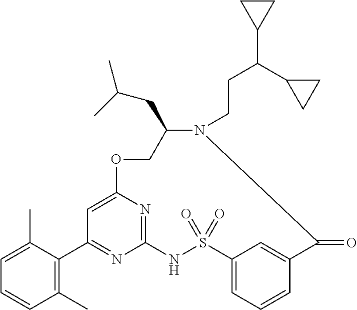 [SMILES: Cc1cccc(C)c1-c1cc2nc(n1)NS(=O)(=O)c1cccc(c1)C(=O)N(CCC(C1CC1)C1CC1)[C@H](CC(C)C)CO2] | | | +++ | +++ | | |
| 982 | 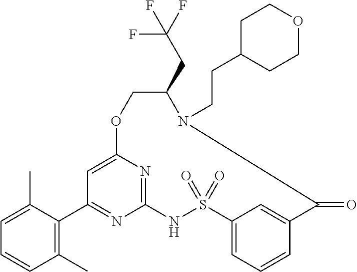 [SMILES: Cc1cccc(C)c1-c1cc2nc(n1)NS(=O)(=O)c1cccc(c1)C(=O)N(CCC1CCOCC1)[C@H](CC(F)(F)F)CO2] | | | ++ | ++ | | |
| 983 | 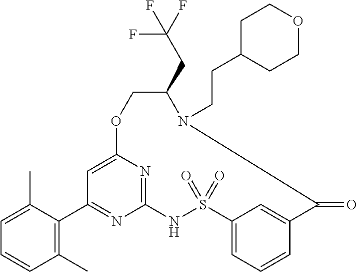 [SMILES: Cc1cccc(C)c1-c1cc2nc(n1)NS(=O)(=O)c1cccc(c1)C(=O)N(CCC1CCOCC1)[C@H](CC(F)(F)F)CO2] | | | +++ | ++ | | |
| 94 | 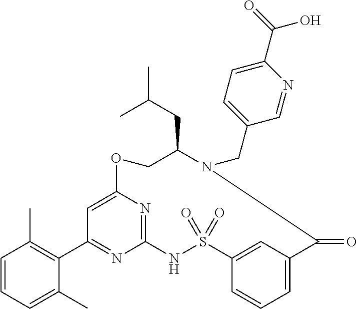 [SMILES: Cc1cccc(C)c1-c1cc2nc(n1)NS(=O)(=O)c1cccc(c1)C(=O)N(Cc1ccc(C(=O)O)nc1)[C@H](CC(C)C)CO2] | | | ++ | +++ | | |
| 1010 | 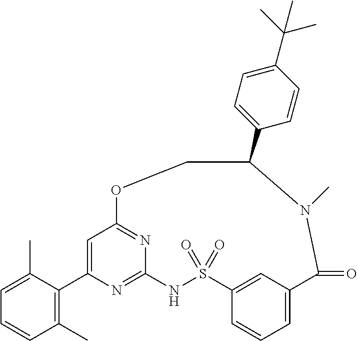 [SMILES: Cc1cccc(C)c1-c1cc2nc(n1)NS(=O)(=O)c1cccc(c1)C(=O)N(C)[C@H](c1ccc(C(C)(C)C)cc1)CO2] | | | +++ | ++ | | |
| 1011 | 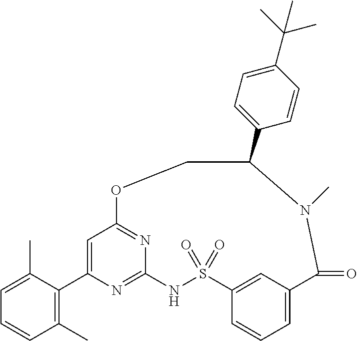 [SMILES: Cc1cccc(C)c1-c1cc2nc(n1)NS(=O)(=O)c1cccc(c1)C(=O)N(C)[C@H](c1ccc(C(C)(C)C)cc1)CO2] | | | +++ | +++ | | |
| 1106 | 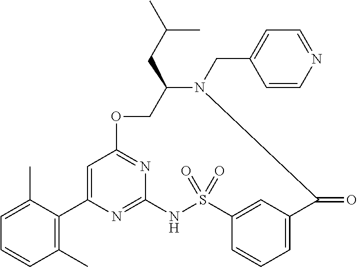 [SMILES: Cc1cccc(C)c1-c1cc2nc(n1)NS(=O)(=O)c1cccc(c1)C(=O)N(Cc1ccncc1)[C@H](CC(C)C)CO2] | | | ++ | +++ | ++ | ++ |
| 84 | 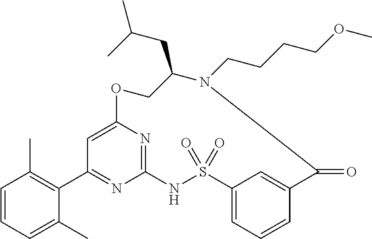 [SMILES: COCCCCN1C(=O)c2cccc(c2)S(=O)(=O)Nc2nc(cc(-c3c(C)cccc3C)n2)OC[C@H]1CC(C)C] | | | +++ | +++ | +++ | +++ |
| 1105 | 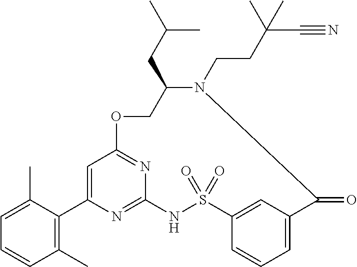 [SMILES: Cc1cccc(C)c1-c1cc2nc(n1)NS(=O)(=O)c1cccc(c1)C(=O)N(CCC(C)(C)C#N)[C@H](CC(C)C)CO2] | | | | | +++ | +++ |
| 1104 | 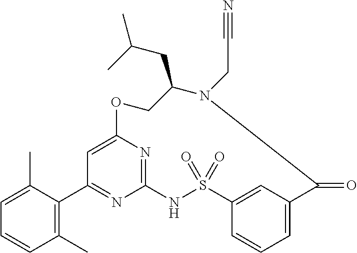 [SMILES: Cc1cccc(C)c1-c1cc2nc(n1)NS(=O)(=O)c1cccc(c1)C(=O)N(CC#N)[C@H](CC(C)C)CO2] | | | | | +++ | +++ |
| 1103 | 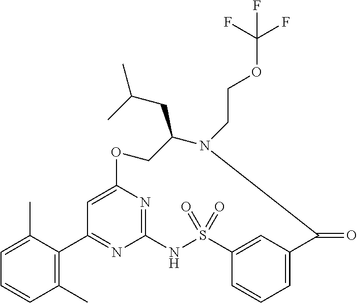 [SMILES: Cc1cccc(C)c1-c1cc2nc(n1)NS(=O)(=O)c1cccc(c1)C(=O)N(CCOC(F)(F)F)[C@H](CC(C)C)CO2] | | | | | +++ | +++ |
| 412 | 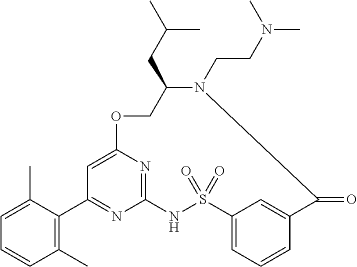 [SMILES: Cc1cccc(C)c1-c1cc2nc(n1)NS(=O)(=O)c1cccc(c1)C(=O)N(CCN(C)C)[C@H](CC(C)C)CO2] | | | | | +++ | +++ |
| 411 | 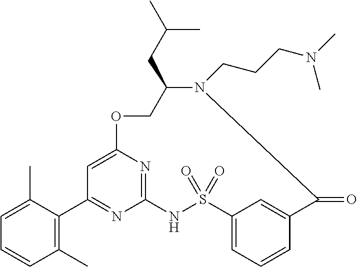 [SMILES: Cc1cccc(C)c1-c1cc2nc(n1)NS(=O)(=O)c1cccc(c1)C(=O)N(CCCN(C)C)[C@H](CC(C)C)CO2] | | | | | +++ | +++ |
| 410 | 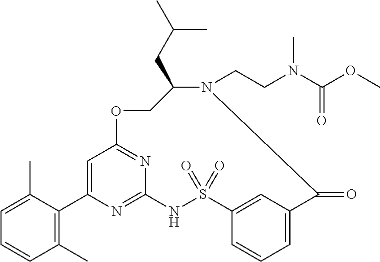 [SMILES: COC(=O)N(C)CCN1C(=O)c2cccc(c2)S(=O)(=O)Nc2nc(cc(-c3c(C)cccc3C)n2)OC[C@H]1CC(C)C] | | | | | +++ | +++ |
| 91 | 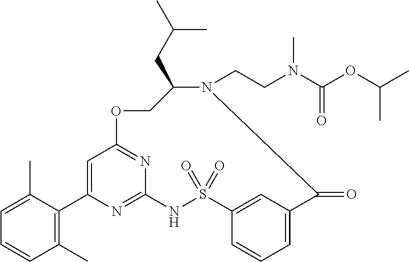 [SMILES: Cc1cccc(C)c1-c1cc2nc(n1)NS(=O)(=O)c1cccc(c1)C(=O)N(CCN(C)C(=O)OC(C)C)[C@H](CC(C)C)CO2] | | | | | +++ | +++ |
| 409 | 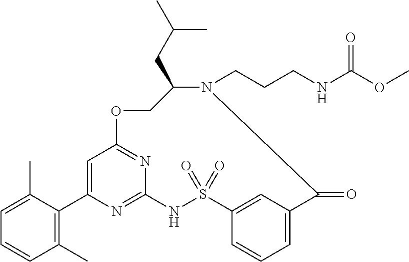 [SMILES: COC(=O)NCCCN1C(=O)c2cccc(c2)S(=O)(=O)Nc2nc(cc(-c3c(C)cccc3C)n2)OC[C@H]1CC(C)C] | | | | | +++ | +++ |
| 408 | 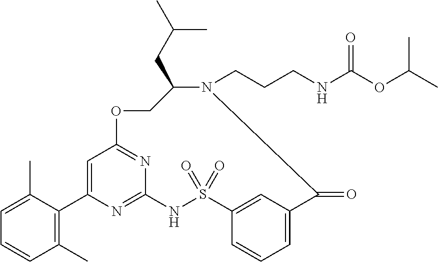 [SMILES: Cc1cccc(C)c1-c1cc2nc(n1)NS(=O)(=O)c1cccc(c1)C(=O)N(CCCNC(=O)OC(C)C)[C@H](CC(C)C)CO2] | | | | | +++ | +++ |
| 407 | 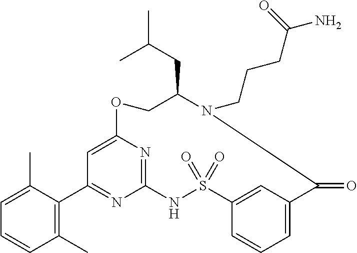 [SMILES: Cc1cccc(C)c1-c1cc2nc(n1)NS(=O)(=O)c1cccc(c1)C(=O)N(CCCC(N)=O)[C@H](CC(C)C)CO2] | | | | | ++ | +++ |

5. Biological Activity Data

Table 8 represents CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” 00 Activity: +++ is >60%; ++ is 30-60%; + is <30%).

TABLE 8

| | | | 3T3 | Ent. | Ent. A | Ent. | Ent. B |
|---|---|---|---|---|---|---|---|
| | | 3T3 | Max | A | Max | B | Max |
| Cmpd | | EC₅₀ | Activity | EC₅₀ | Activity | EC₅₀ | Activity |
| Number | Structure | (μM) | (%) | (μM) | (%) | (μM) | (%) |
| 406 | 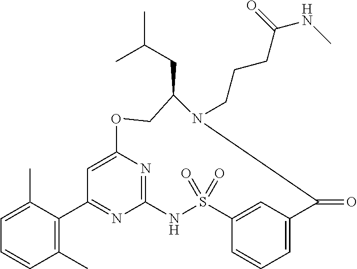 [SMILES: CNC(=O)CCCN1C(=O)c2cccc(c2)S(=O)(=O)Nc2nc(cc(-c3c(C)cccc3C)n2)OC[C@H]1CC(C)C] | | | | | ++ | +++ |
| 92 | 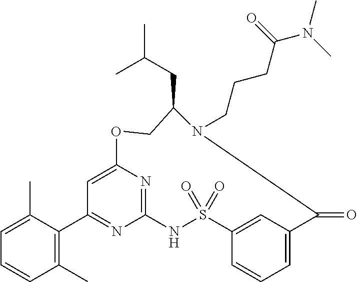 [SMILES: Cc1cccc(C)c1-c1cc2nc(n1)NS(=O)(=O)c1cccc(c1)C(=O)N(CCCC(=O)N(C)C)[C@H](CC(C)C)CO2] | | | | | +++ | +++ |
| 405 | 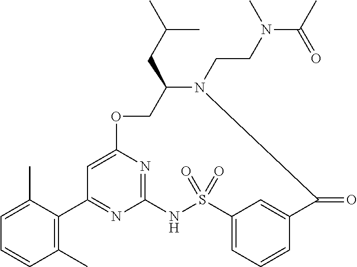 [SMILES: CC(=O)N(C)CCN1C(=O)c2cccc(c2)S(=O)(=O)Nc2nc(cc(-c3c(C)cccc3C)n2)OC[C@H]1CC(C)C] | | | | | +++ | +++ |
| 404 | 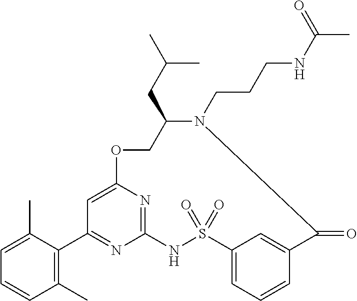 [SMILES: CC(=O)NCCCN1C(=O)c2cccc(c2)S(=O)(=O)Nc2nc(cc(-c3c(C)cccc3C)n2)OC[C@H]1CC(C)C] | | | | | +++ | +++ |
| 96 | 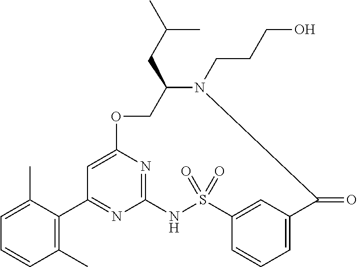 [SMILES: Cc1cccc(C)c1-c1cc2nc(n1)NS(=O)(=O)c1cccc(c1)C(=O)N(CCCO)[C@H](CC(C)C)CO2] | | | | | +++ | +++ |
| 336 | 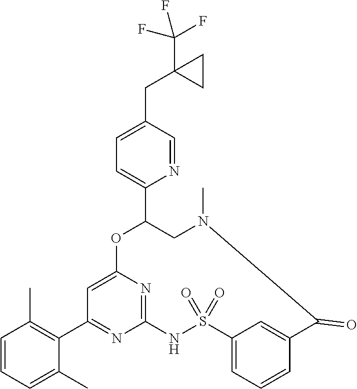 [SMILES: Cc1cccc(C)c1-c1cc2nc(n1)NS(=O)(=O)c1cccc(c1)C(=O)N(C)CC(c1ccc(CC3(C(F)(F)F)CC3)cn1)O2] | | | | | +++ | +++ |
| 980 | 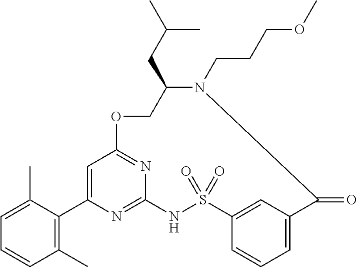 [SMILES: COCCCN1C(=O)c2cccc(c2)S(=O)(=O)Nc2nc(cc(-c3c(C)cccc3C)n2)OC[C@H]1CC(C)C] | | | | | +++ | +++ |
| 46 | 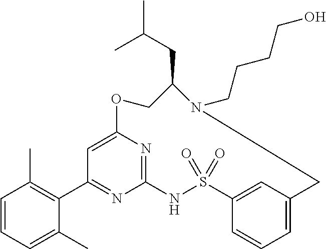 [SMILES: Cc1cccc(C)c1-c1cc2nc(n1)NS(=O)(=O)c1cccc(c1)CN(CCCCO)[C@H](CC(C)C)CO2] | | | | | +++ | +++ |
| 104 | 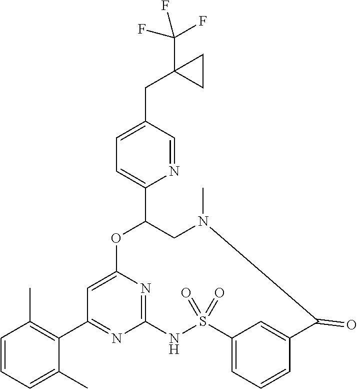 [SMILES: Cc1cccc(C)c1-c1cc2nc(n1)NS(=O)(=O)c1cccc(c1)C(=O)N(C)CC(c1ccc(CC3(C(F)(F)F)CC3)cn1)O2] | | | | | +++ | +++ |
| 105 | 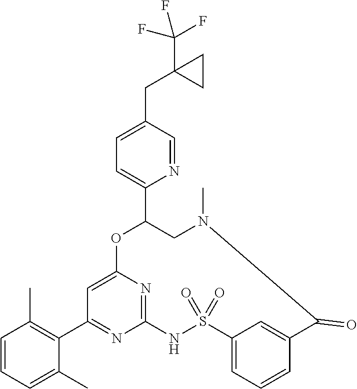 [SMILES: Cc1cccc(C)c1-c1cc2nc(n1)NS(=O)(=O)c1cccc(c1)C(=O)N(C)CC(c1ccc(CC3(C(F)(F)F)CC3)cn1)O2] | | | | | +++ | + |
| 42 | 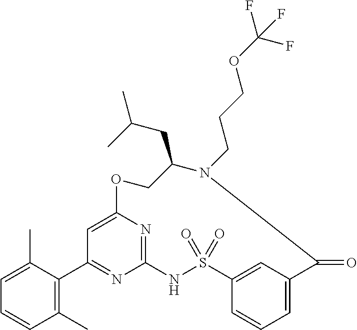 [SMILES: Cc1cccc(C)c1-c1cc2nc(n1)NS(=O)(=O)c1cccc(c1)C(=O)N(CCCOC(F)(F)F)[C@H](CC(C)C)CO2] | | | | | +++ | +++ |
| 1102 | 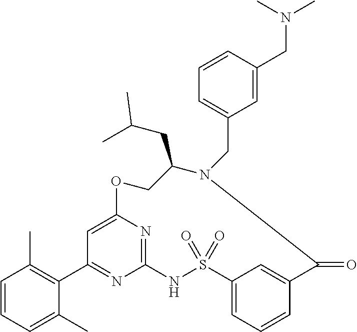 [SMILES: Cc1cccc(C)c1-c1cc2nc(n1)NS(=O)(=O)c1cccc(c1)C(=O)N(Cc1cccc(CN(C)C)c1)[C@H](CC(C)C)CO2] | | | | | +++ | +++ |
| 1100 | 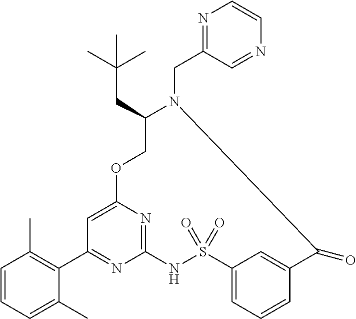 [SMILES: Cc1cccc(C)c1-c1cc2nc(n1)NS(=O)(=O)c1cccc(c1)C(=O)N(Cc1cnccn1)[C@H](CC(C)(C)C)CO2] | | | | | +++ | +++ |
| 113 | 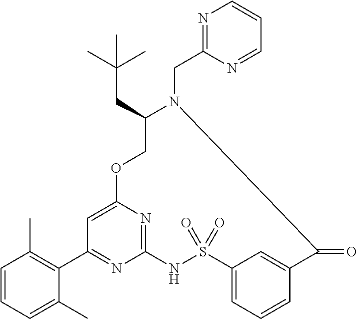 [SMILES: Cc1cccc(C)c1-c1cc2nc(n1)NS(=O)(=O)c1cccc(c1)C(=O)N(Cc1ncccn1)[C@H](CC(C)(C)C)CO2] | | | | | +++ | +++ |
| 1099 | 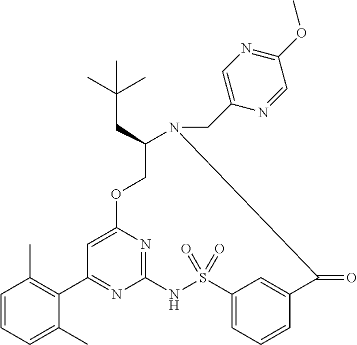 [SMILES: COc1cnc(CN2C(=O)c3cccc(c3)S(=O)(=O)Nc3nc(cc(-c4c(C)cccc4C)n3)OC[C@H]2CC(C)(C)C)cn1] | | | | | +++ | +++ |
| 1101 | 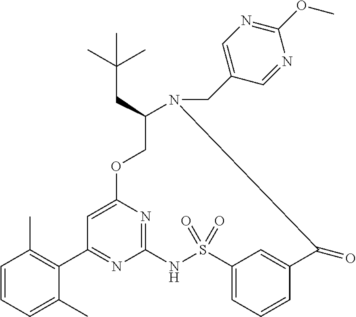 [SMILES: COc1ncc(CN2C(=O)c3cccc(c3)S(=O)(=O)Nc3nc(cc(-c4c(C)cccc4C)n3)OC[C@H]2CC(C)(C)C)cn1] | | | | | +++ | +++ |
| 1098 | 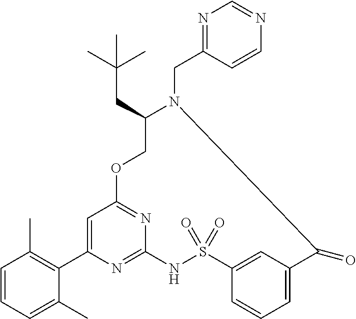 [SMILES: Cc1cccc(C)c1-c1cc2nc(n1)NS(=O)(=O)c1cccc(c1)C(=O)N(Cc1ccncn1)[C@H](CC(C)(C)C)CO2] | | | | | +++ | +++ |
| 135 | 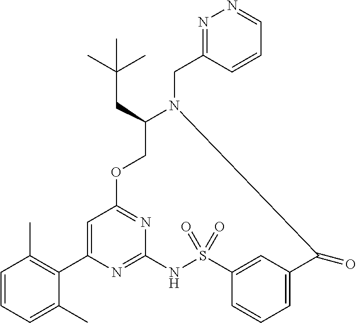 [SMILES: Cc1cccc(C)c1-c1cc2nc(n1)NS(=O)(=O)c1cccc(c1)C(=O)N(Cc1cccnn1)[C@H](CC(C)(C)C)CO2] | | | | | +++ | +++ |
| 1097 | 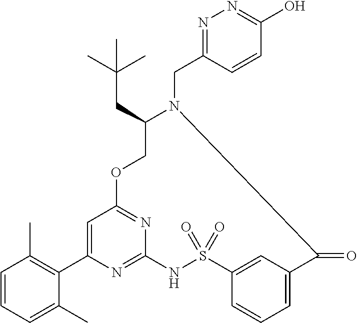 [SMILES: Cc1cccc(C)c1-c1cc2nc(n1)NS(=O)(=O)c1cccc(c1)C(=O)N(Cc1ccc(O)nn1)[C@H](CC(C)(C)C)CO2] | | | | | +++ | ++ |
| 136 | 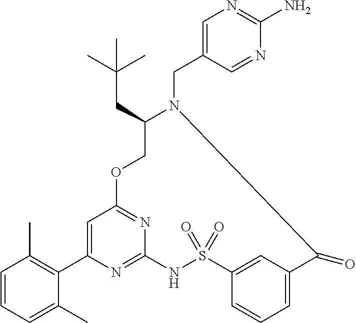 [SMILES: Cc1cccc(C)c1-c1cc2nc(n1)NS(=O)(=O)c1cccc(c1)C(=O)N(Cc1cnc(N)nc1)[C@H](CC(C)(C)C)CO2] | | | | | +++ | +++ |
| 1096 | 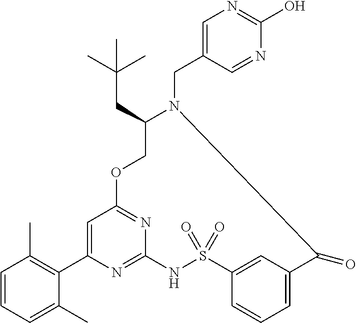 [SMILES: Cc1cccc(C)c1-c1cc2nc(n1)NS(=O)(=O)c1cccc(c1)C(=O)N(Cc1cnc(O)nc1)[C@H](CC(C)(C)C)CO2] | | | | | ND | + |
| 403 | 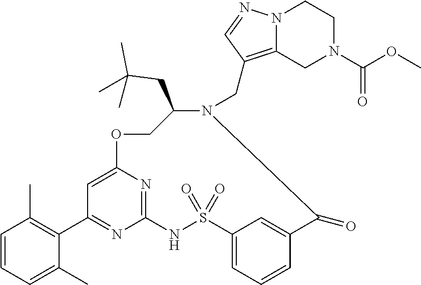 [SMILES: COC(=O)N1CCn2ncc(CN3C(=O)c4cccc(c4)S(=O)(=O)Nc4nc(cc(-c5c(C)cccc5C)n4)OC[C@H]3CC(C)(C)C)c2C1] | | | | | +++ | +++ |
| 1094 | 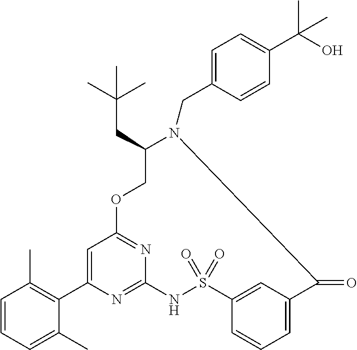 [SMILES: Cc1cccc(C)c1-c1cc2nc(n1)NS(=O)(=O)c1cccc(c1)C(=O)N(Cc1ccc(C(C)(C)O)cc1)[C@H](CC(C)(C)C)CO2] | | | | | +++ | +++ |
| 1093 | 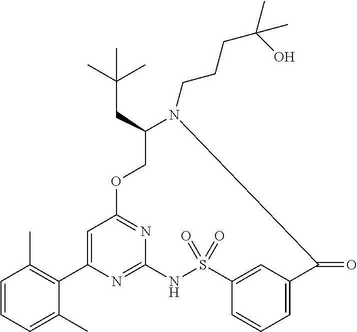 [SMILES: Cc1cccc(C)c1-c1cc2nc(n1)NS(=O)(=O)c1cccc(c1)C(=O)N(CCCC(C)(C)O)[C@H](CC(C)(C)C)CO2] | | | | | +++ | +++ |
| 107 | 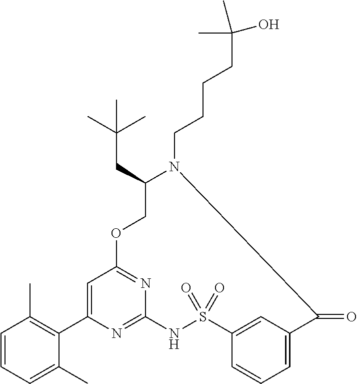 [SMILES: Cc1cccc(C)c1-c1cc2nc(n1)NS(=O)(=O)c1cccc(c1)C(=O)N(CCCCC(C)(C)O)[C@H](CC(C)(C)C)CO2] | | | | | +++ | +++ |
| 1095 | 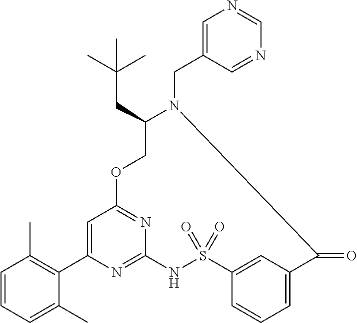 [SMILES: Cc1cccc(C)c1-c1cc2nc(n1)NS(=O)(=O)c1cccc(c1)C(=O)N(Cc1cncnc1)[C@H](CC(C)(C)C)CO2] | | | | | +++ | +++ |
| 1092 | 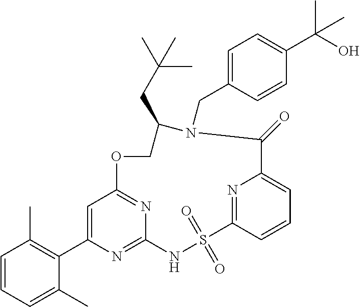 [SMILES: Cc1cccc(C)c1-c1cc2nc(n1)NS(=O)(=O)c1cccc(n1)C(=O)N(Cc1ccc(C(C)(C)O)cc1)[C@H](CC(C)(C)C)CO2] | | | | | +++ | +++ |
| 1091 | 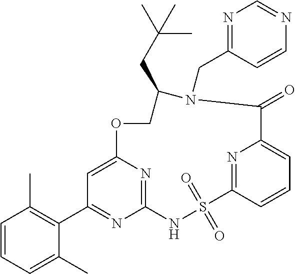 [SMILES: Cc1cccc(C)c1-c1cc2nc(n1)NS(=O)(=O)c1cccc(n1)C(=O)N(Cc1ccncn1)[C@H](CC(C)(C)C)CO2] | | | | | +++ | +++ |
| 75 | 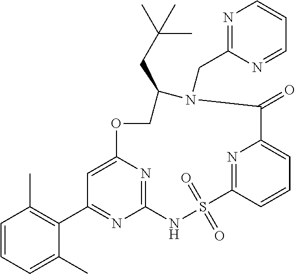 [SMILES: Cc1cccc(C)c1-c1cc2nc(n1)NS(=O)(=O)c1cccc(n1)C(=O)N(Cc1ncccn1)[C@H](CC(C)(C)C)CO2] | | | | | +++ | +++ |
| 1090 | 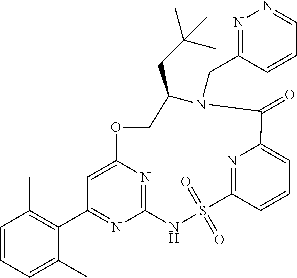 [SMILES: Cc1cccc(C)c1-c1cc2nc(n1)NS(=O)(=O)c1cccc(n1)C(=O)N(Cc1cccnn1)[C@H](CC(C)(C)C)CO2] | | | | | +++ | +++ |
| 402 | 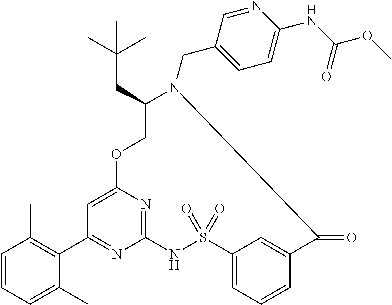 [SMILES: COC(=O)Nc1ccc(CN2C(=O)c3cccc(c3)S(=O)(=O)Nc3nc(cc(-c4c(C)cccc4C)n3)OC[C@H]2CC(C)(C)C)cn1] | | | | | +++ | +++ |
| 401 | 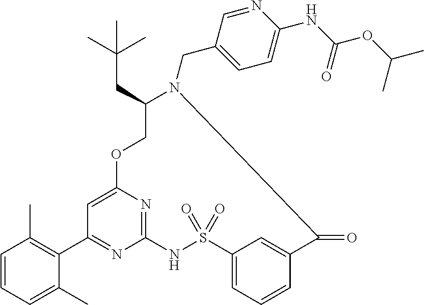 [SMILES: Cc1cccc(C)c1-c1cc2nc(n1)NS(=O)(=O)c1cccc(c1)C(=O)N(Cc1ccc(NC(=O)OC(C)C)nc1)[C@H](CC(C)(C)C)CO2] | | | | | +++ | +++ |
| 508 | 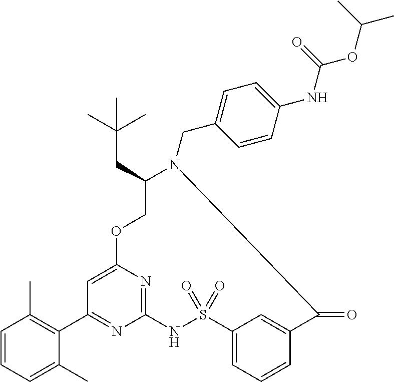 [SMILES: Cc1cccc(C)c1-c1cc2nc(n1)NS(=O)(=O)c1cccc(c1)C(=O)N(Cc1ccc(NC(=O)OC(C)C)cc1)[C@H](CC(C)(C)C)CO2] | | | | | +++ | +++ |
| 76 | 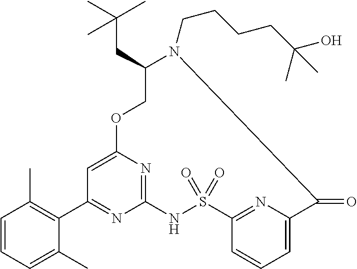 [SMILES: Cc1cccc(C)c1-c1cc2nc(n1)NS(=O)(=O)c1cccc(n1)C(=O)N(CCCCC(C)(C)O)[C@H](CC(C)(C)C)CO2] | | | | | +++ | +++ |
| 849 | 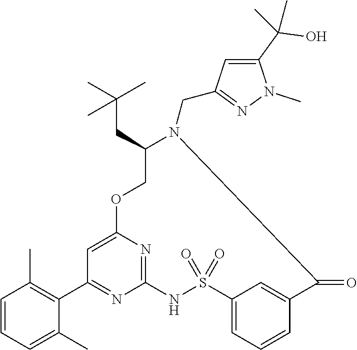 [SMILES: Cc1cccc(C)c1-c1cc2nc(n1)NS(=O)(=O)c1cccc(c1)C(=O)N(Cc1cc(C(C)(C)O)n(C)n1)[C@H](CC(C)(C)C)CO2] | | | | | +++ | +++ |
| 1089 | 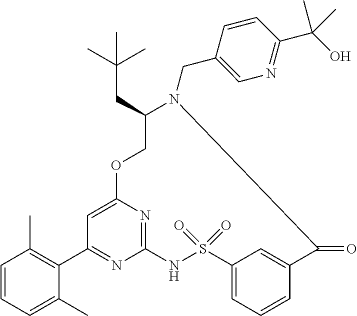 [SMILES: Cc1cccc(C)c1-c1cc2nc(n1)NS(=O)(=O)c1cccc(c1)C(=O)N(Cc1ccc(C(C)(C)O)nc1)[C@H](CC(C)(C)C)CO2] | | | | | +++ | +++ |
| 1085 | 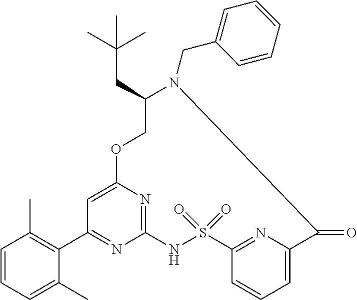 [SMILES: Cc1cccc(C)c1-c1cc2nc(n1)NS(=O)(=O)c1cccc(n1)C(=O)N(Cc1ccccc1)[C@H](CC(C)(C)C)CO2] | | | | | +++ | +++ |
| 1083 | 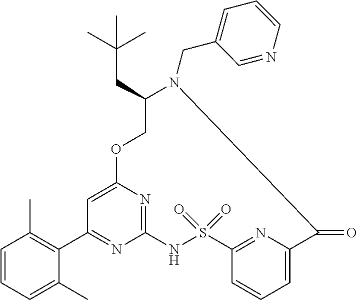 [SMILES: Cc1cccc(C)c1-c1cc2nc(n1)NS(=O)(=O)c1cccc(n1)C(=O)N(Cc1cccnc1)[C@H](CC(C)(C)C)CO2] | | | | | +++ | +++ |
| 1087 | 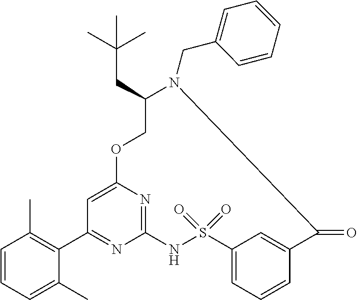 [SMILES: Cc1cccc(C)c1-c1cc2nc(n1)NS(=O)(=O)c1cccc(c1)C(=O)N(Cc1ccccc1)[C@H](CC(C)(C)C)CO2] | | | | | +++ | +++ |
| 1084 | 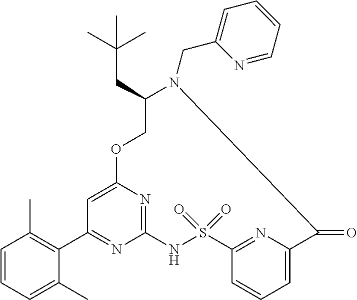 [SMILES: Cc1cccc(C)c1-c1cc2nc(n1)NS(=O)(=O)c1cccc(n1)C(=O)N(Cc1ccccn1)[C@H](CC(C)(C)C)CO2] | | | | | +++ | +++ |
| 124 | 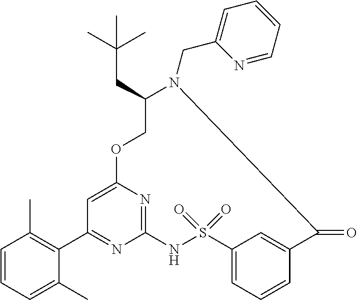 [SMILES: Cc1cccc(C)c1-c1cc2nc(n1)NS(=O)(=O)c1cccc(c1)C(=O)N(Cc1ccccn1)[C@H](CC(C)(C)C)CO2] | | | | | +++ | +++ |
| 1086 | 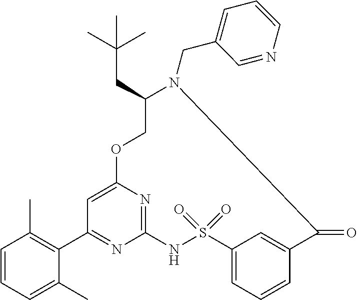 [SMILES: Cc1cccc(C)c1-c1cc2nc(n1)NS(=O)(=O)c1cccc(c1)C(=O)N(Cc1cccnc1)[C@H](CC(C)(C)C)CO2] | | | | | +++ | +++ |
| 507 | 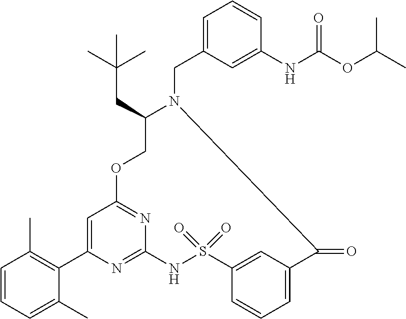 [SMILES: Cc1cccc(C)c1-c1cc2nc(n1)NS(=O)(=O)c1cccc(c1)C(=O)N(Cc1cccc(NC(=O)OC(C)C)c1)[C@H](CC(C)(C)C)CO2] | | | | | +++ | +++ |
| 506 | 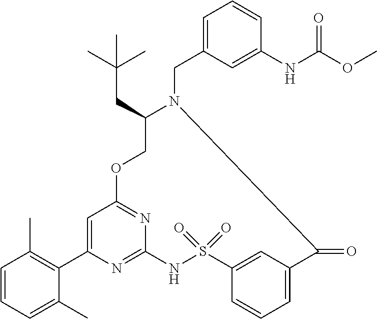 [SMILES: COC(=O)Nc1cccc(CN2C(=O)c3cccc(c3)S(=O)(=O)Nc3nc(cc(-c4c(C)cccc4C)n3)OC[C@H]2CC(C)(C)C)c1] | | | | | +++ | +++ |
| 1082 | 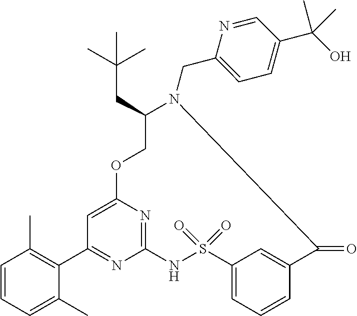 [SMILES: Cc1cccc(C)c1-c1cc2nc(n1)NS(=O)(=O)c1cccc(c1)C(=O)N(Cc1ccc(C(C)(C)O)cn1)[C@H](CC(C)(C)C)CO2] | | | | | +++ | +++ |
| 1088 | 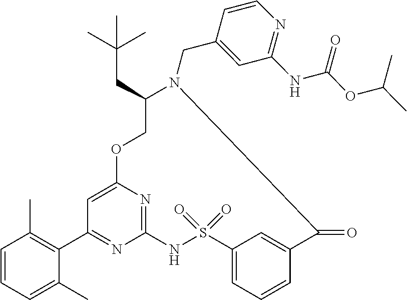 [SMILES: Cc1cccc(C)c1-c1cc2nc(n1)NS(=O)(=O)c1cccc(c1)C(=O)N(Cc1ccnc(NC(=O)OC(C)C)c1)[C@H](CC(C)(C)C)CO2] | | | | | +++ | +++ |
| 335 | 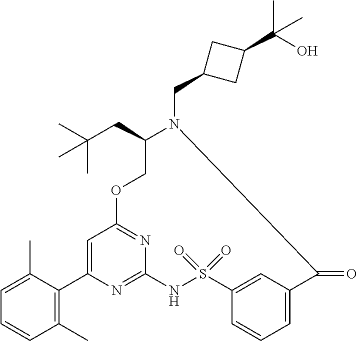 [SMILES: Cc1cccc(C)c1-c1cc2nc(n1)NS(=O)(=O)c1cccc(c1)C(=O)N(C[C@H]1C[C@@H](C(C)(C)O)C1)[C@H](CC(C)(C)C)CO2] | | | | | +++ | +++ |
| 334 | 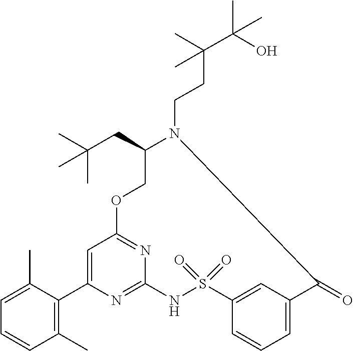 [SMILES: Cc1cccc(C)c1-c1cc2nc(n1)NS(=O)(=O)c1cccc(c1)C(=O)N(CCC(C)(C)C(C)(C)O)[C@H](CC(C)(C)C)CO2] | | | | | +++ | +++ |
| 664 | 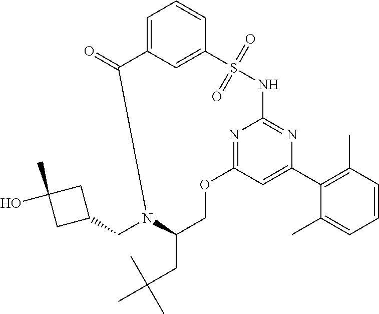 | | | | | +++ | +++ |
| 665 | 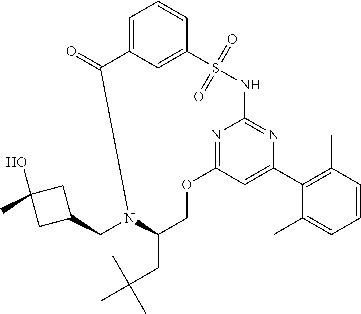 | | | | | +++ | +++ |
| 149 | 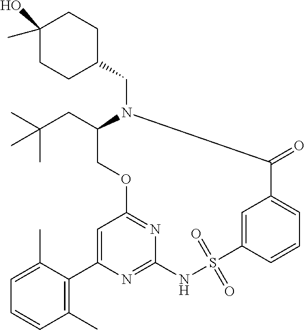 | | | | | +++ | +++ |
| 150 | 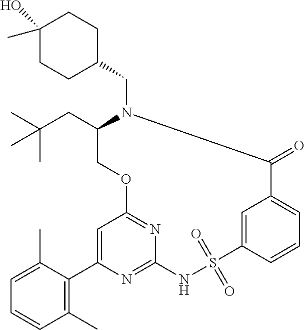 | | | | | +++ | +++ |
| 1020 | 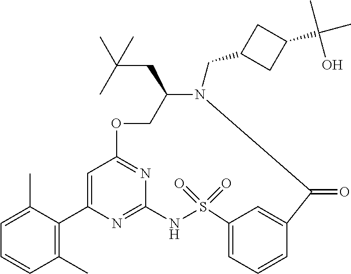 | | | | | +++ | +++ |
| 663 | 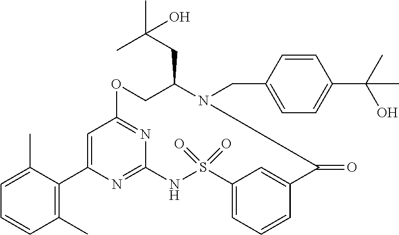 [SMILES: Cc1cccc(C)c1-c1cc2nc(n1)NS(=O)(=O)c1cccc(c1)C(=O)N(Cc1ccc(C(C)(C)O)cc1)[C@H](CC(C)(C)O)CO2] | | | | | +++ | +++ |
| 662 | 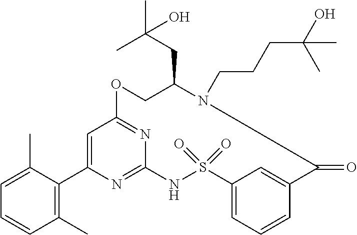 [SMILES: Cc1cccc(C)c1-c1cc2nc(n1)NS(=O)(=O)c1cccc(c1)C(=O)N(CCCC(C)(C)O)[C@H](CC(C)(C)O)CO2] | | | | | ++ | + |
| 660 | 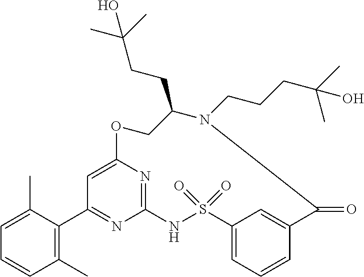 [SMILES: Cc1cccc(C)c1-c1cc2nc(n1)NS(=O)(=O)c1cccc(c1)C(=O)N(CCCC(C)(C)O)[C@H](CCC(C)(C)O)CO2] | | | | | ND | + |
| 661 | 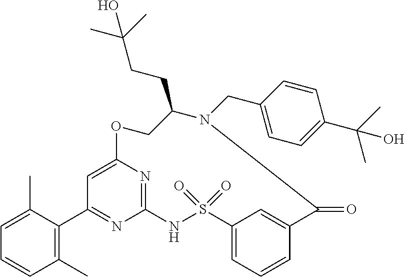 [SMILES: Cc1cccc(C)c1-c1cc2nc(n1)NS(=O)(=O)c1cccc(c1)C(=O)N(Cc1ccc(C(C)(C)O)cc1)[C@H](CCC(C)(C)O)CO2] | | | | | ++ | ++ |
| 659 | 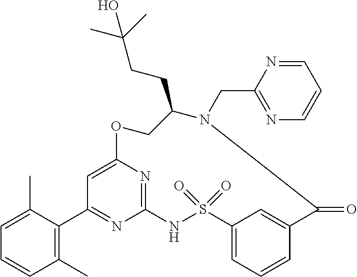 [SMILES: Cc1cccc(C)c1-c1cc2nc(n1)NS(=O)(=O)c1cccc(c1)C(=O)N(Cc1ncccn1)[C@H](CCC(C)(C)O)CO2] | | | | | ++ | ++ |
| 808 | 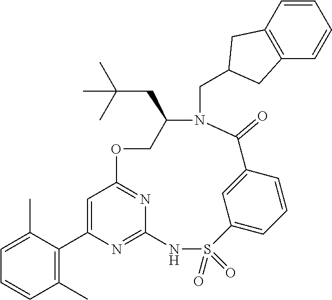 [SMILES: Cc1cccc(C)c1-c1cc2nc(n1)NS(=O)(=O)c1cccc(c1)C(=O)N(CC1Cc3ccccc3C1)[C@H](CC(C)(C)C)CO2] | | | | | +++ | +++ |
| 658 | 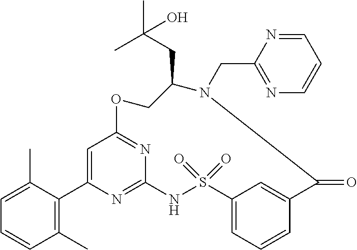 [SMILES: Cc1cccc(C)c1-c1cc2nc(n1)NS(=O)(=O)c1cccc(c1)C(=O)N(Cc1ncccn1)[C@H](CC(C)(C)O)CO2] | | | | | ++ | +++ |
| 934 | 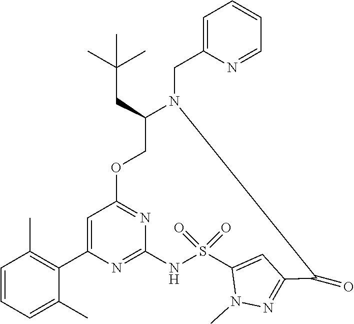 [SMILES: Cc1cccc(C)c1-c1cc2nc(n1)NS(=O)(=O)c1cc(nn1C)C(=O)N(Cc1ccccn1)[C@H](CC(C)(C)C)CO2] | | | | | +++ | ++ |
| 933 | 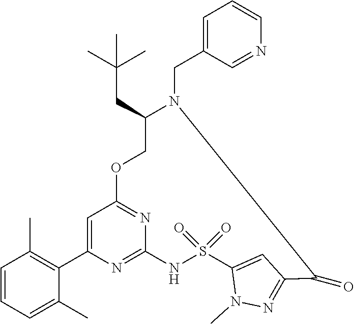 [SMILES: Cc1cccc(C)c1-c1cc2nc(n1)NS(=O)(=O)c1cc(nn1C)C(=O)N(Cc1cccnc1)[C@H](CC(C)(C)C)CO2] | | | | | ND | + |
| 70 | 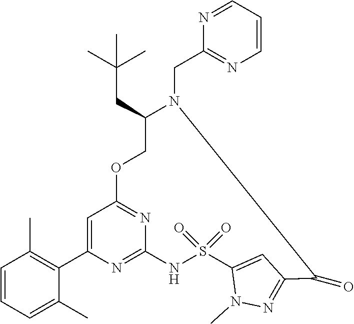 [SMILES: Cc1cccc(C)c1-c1cc2nc(n1)NS(=O)(=O)c1cc(nn1C)C(=O)N(Cc1ncccn1)[C@H](CC(C)(C)C)CO2] | | | | | +++ | ++ |
| 932 | 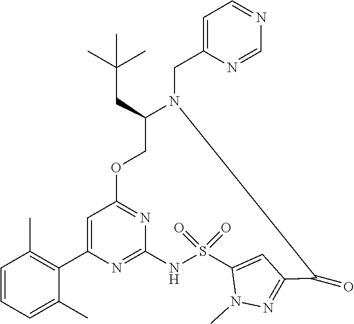 [SMILES: Cc1cccc(C)c1-c1cc2nc(n1)NS(=O)(=O)c1cc(nn1C)C(=O)N(Cc1ccncn1)[C@H](CC(C)(C)C)CO2] | | | | | +++ | + |
| 931 | 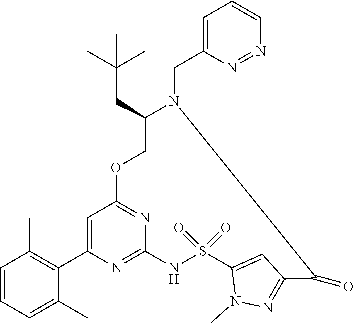 [SMILES: Cc1cccc(C)c1-c1cc2nc(n1)NS(=O)(=O)c1cc(nn1C)C(=O)N(Cc1cccnn1)[C@H](CC(C)(C)C)CO2] | | | | | ++ | + |
| 930 | 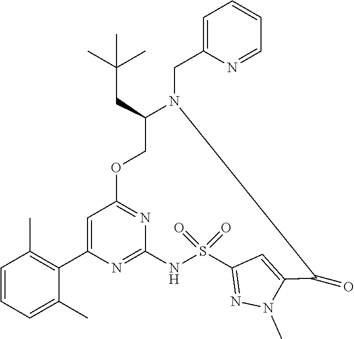 [SMILES: Cc1cccc(C)c1-c1cc2nc(n1)NS(=O)(=O)c1cc(n(C)n1)C(=O)N(Cc1ccccn1)[C@H](CC(C)(C)C)CO2] | | | | | +++ | +++ |
| 929 | 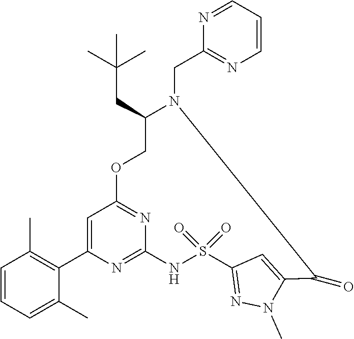 [SMILES: Cc1cccc(C)c1-c1cc2nc(n1)NS(=O)(=O)c1cc(n(C)n1)C(=O)N(Cc1ncccn1)[C@H](CC(C)(C)C)CO2] | | | | | +++ | +++ |
| 43 | 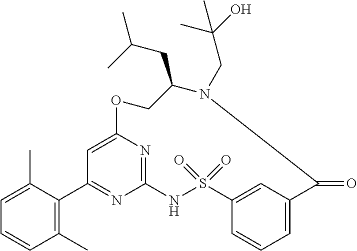 [SMILES: Cc1cccc(C)c1-c1cc2nc(n1)NS(=O)(=O)c1cccc(c1)C(=O)N(CC(C)(C)O)[C@H](CC(C)C)CO2] | | | | | +++ | +++ |
| 1079 | 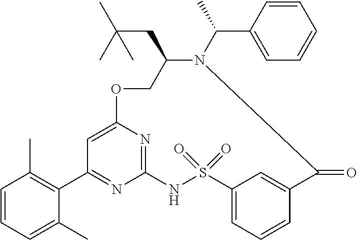 [SMILES: Cc1cccc(C)c1-c1cc2nc(n1)NS(=O)(=O)c1cccc(c1)C(=O)N([C@H](C)c1ccccc1)[C@H](CC(C)(C)C)CO2] | | | | | +++ | +++ |
| 1080 | 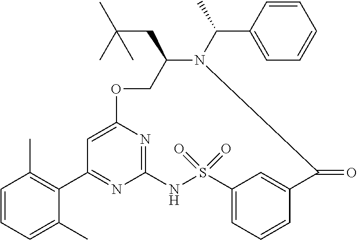 [SMILES: Cc1cccc(C)c1-c1cc2nc(n1)NS(=O)(=O)c1cccc(c1)C(=O)N([C@H](C)c1ccccc1)[C@H](CC(C)(C)C)CO2] | | | | | +++ | +++ |
| 927 | 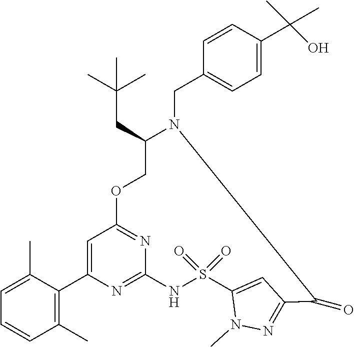 [SMILES: Cc1cccc(C)c1-c1cc2nc(n1)NS(=O)(=O)c1cc(nn1C)C(=O)N(Cc1ccc(C(C)(C)O)cc1)[C@H](CC(C)(C)C)CO2] | | | | | +++ | ++ |
| 928 | 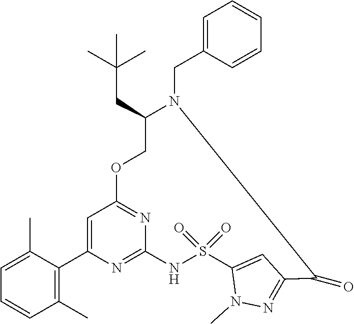 [SMILES: Cc1cccc(C)c1-c1cc2nc(n1)NS(=O)(=O)c1cc(nn1C)C(=O)N(Cc1ccccc1)[C@H](CC(C)(C)C)CO2] | | | | | +++ | ++ |
| 848 | 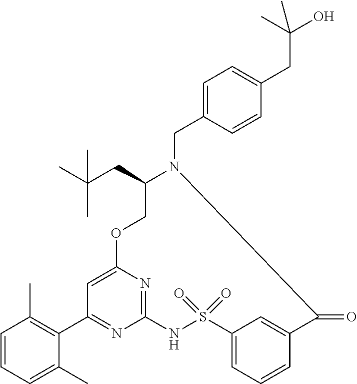 [SMILES: Cc1cccc(C)c1-c1cc2nc(n1)NS(=O)(=O)c1cccc(c1)C(=O)N(Cc1ccc(CC(C)(C)O)cc1)[C@H](CC(C)(C)C)CO2] | | | | | +++ | +++ |
| 398 | 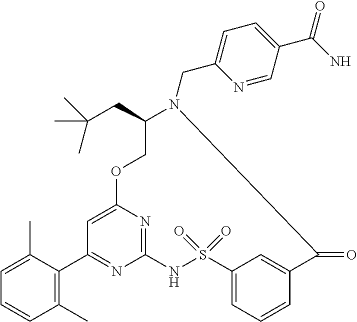 [SMILES: Cc1cccc(C)c1-c1cc2nc(n1)NS(=O)(=O)c1cccc(c1)C(=O)N(Cc1ccc(C(N)=O)cn1)[C@H](CC(C)(C)C)CO2] | | | | | +++ | +++ |
| 397 | 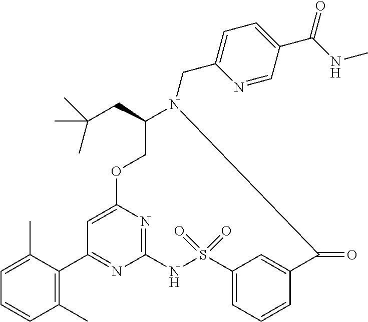 [SMILES: CNC(=O)c1ccc(CN2C(=O)c3cccc(c3)S(=O)(=O)Nc3nc(cc(-c4c(C)cccc4C)n3)OC[C@H]2CC(C)(C)C)nc1] | | | | | +++ | +++ |
| 1110 | 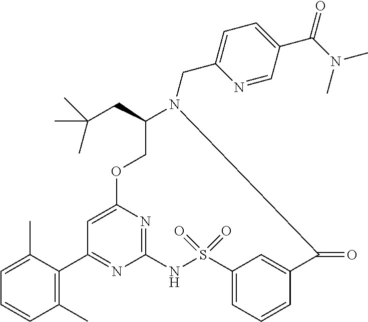 [SMILES: Cc1cccc(C)c1-c1cc2nc(n1)NS(=O)(=O)c1cccc(c1)C(=O)N(Cc1ccc(C(=O)N(C)C)cn1)[C@H](CC(C)(C)C)CO2] | | | | | +++ | +++ |
| 396 | 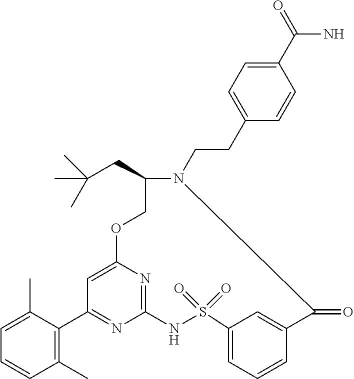 [SMILES: Cc1cccc(C)c1-c1cc2nc(n1)NS(=O)(=O)c1cccc(c1)C(=O)N(CCc1ccc(C(N)=O)cc1)[C@H](CC(C)(C)C)CO2] | | | | | +++ | ++ |
| 395 | 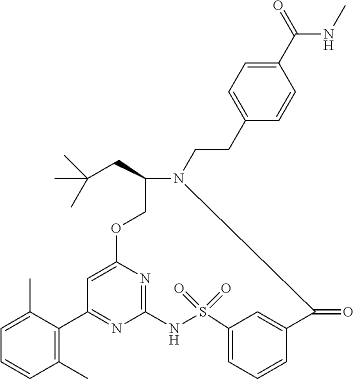 [SMILES: CNC(=O)c1ccc(CCN2C(=O)c3cccc(c3)S(=O)(=O)Nc3nc(cc(-c4c(C)cccc4C)n3)OC[C@H]2CC(C)(C)C)cc1] | | | | | +++ | ++ |
| 394 | 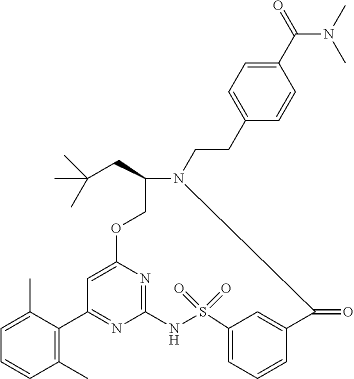 [SMILES: Cc1cccc(C)c1-c1cc2nc(n1)NS(=O)(=O)c1cccc(c1)C(=O)N(CCc1ccc(C(=O)N(C)C)cc1)[C@H](CC(C)(C)C)CO2] | | | | | +++ | +++ |
| 400 | 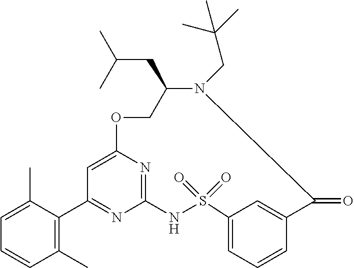 [SMILES: Cc1cccc(C)c1-c1cc2nc(n1)NS(=O)(=O)c1cccc(c1)C(=O)N(CC(C)(C)C)[C@H](CC(C)C)CO2] | | | | | +++ | ++ |
| 926 | 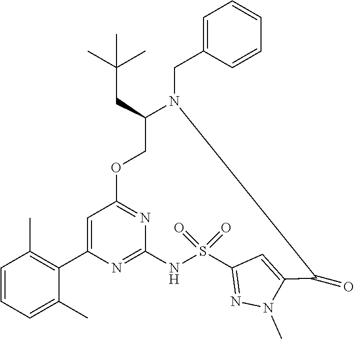 [SMILES: Cc1cccc(C)c1-c1cc2nc(n1)NS(=O)(=O)c1cc(n(C)n1)C(=O)N(Cc1ccccc1)[C@H](CC(C)(C)C)CO2] | | | | | + | ++ |
| 923 | 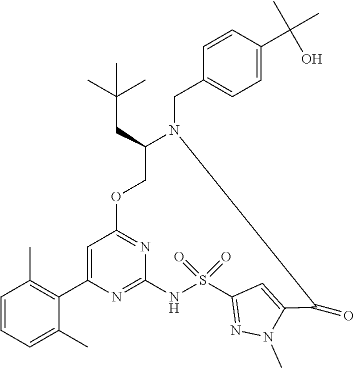 [SMILES: Cc1cccc(C)c1-c1cc2nc(n1)NS(=O)(=O)c1cc(n(C)n1)C(=O)N(Cc1ccc(C(C)(C)O)cc1)[C@H](CC(C)(C)C)CO2] | | | | | +++ | +++ |
| 922 | 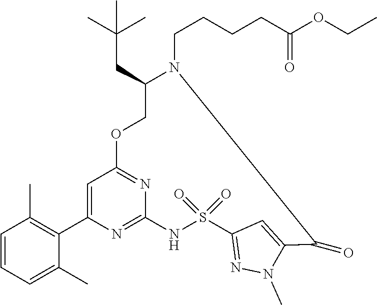 [SMILES: CCOC(=O)CCCCN1C(=O)c2cc(nn2C)S(=O)(=O)Nc2nc(cc(-c3c(C)cccc3C)n2)OC[C@H]1CC(C)(C)C] | | | | | + | ++ |
| 921 | 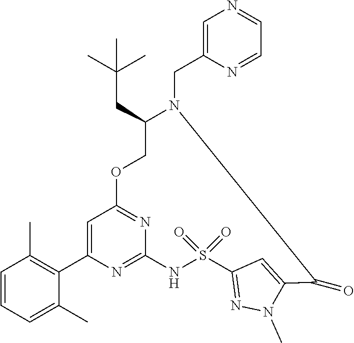 [SMILES: Cc1cccc(C)c1-c1cc2nc(n1)NS(=O)(=O)c1cc(n(C)n1)C(=O)N(Cc1cnccn1)[C@H](CC(C)(C)C)CO2] | | | | | +++ | +++ |
| 925 | 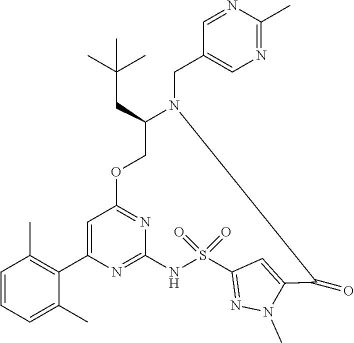 [SMILES: Cc1ncc(CN2C(=O)c3cc(nn3C)S(=O)(=O)Nc3nc(cc(-c4c(C)cccc4C)n3)OC[C@H]2CC(C)(C)C)cn1] | | | | | +++ | ++ |

6. Biological Activity Data

Table 9 represents CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” 00 Activity: +++ is >600%; ++ is 30-60%; + is <300%).

TABLE 9

| | | | 3T3 | Ent. | Ent. A | | Ent. B |
|---|---|---|---|---|---|---|---|
| | | 3T3 | Max | A | Max | Ent. B | Max |
| Cmpd | | EC₅₀ | Activity | EC₅₀ | Activity | EC₅₀ | Activity |
| Number | Structure | (μM) | (%) | (μM) | (%) | (μM) | (%) |
| 924 | 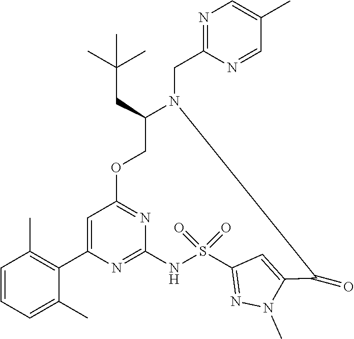 [SMILES: Cc1cnc(CN2C(=O)c3cc(nn3C)S(=O)(=O)Nc3nc(cc(-c4c(C)cccc4C)n3)OC[C@H]2CC(C)(C)C)nc1] | | | | | +++ | +++ |
| 847 | 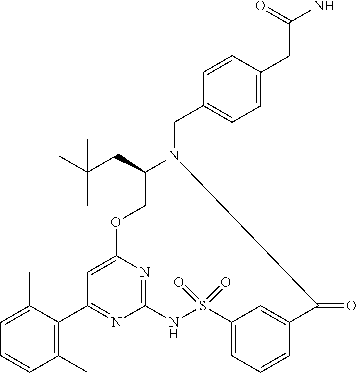 [SMILES: Cc1cccc(C)c1-c1cc2nc(n1)NS(=O)(=O)c1cccc(c1)C(=O)N(Cc1ccc(CC(N)=O)cc1)[C@H](CC(C)(C)C)CO2] | | | | | +++ | +++ |
| 846 | 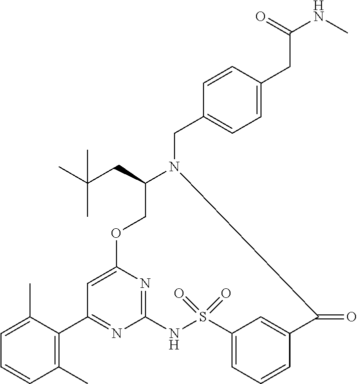 [SMILES: CNC(=O)Cc1ccc(CN2C(=O)c3cccc(c3)S(=O)(=O)Nc3nc(cc(-c4c(C)cccc4C)n3)OC[C@H]2CC(C)(C)C)cc1] | | | | | +++ | +++ |
| 845 | 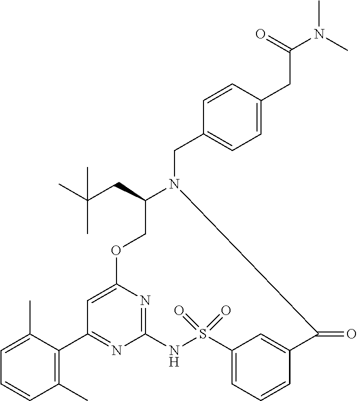 [SMILES: Cc1cccc(C)c1-c1cc2nc(n1)NS(=O)(=O)c1cccc(c1)C(=O)N(Cc1ccc(CC(=O)N(C)C)cc1)[C@H](CC(C)(C)C)CO2] | | | | | +++ | +++ |
| 390 | 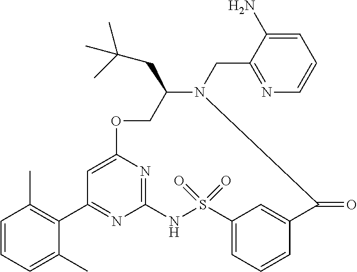 [SMILES: Cc1cccc(C)c1-c1cc2nc(n1)NS(=O)(=O)c1cccc(c1)C(=O)N(Cc1ncccc1N)[C@H](CC(C)(C)C)CO2] | | | | | +++ | +++ |
| 393 | 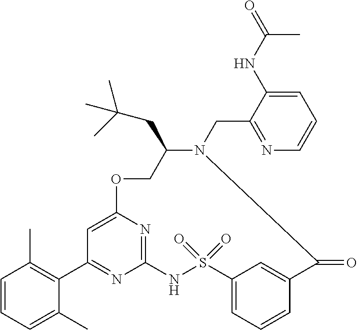 [SMILES: CC(=O)Nc1cccnc1CN1C(=O)c2cccc(c2)S(=O)(=O)Nc2nc(cc(-c3c(C)cccc3C)n2)OC[C@H]1CC(C)(C)C] | | | | | +++ | +++ |
| 392 | 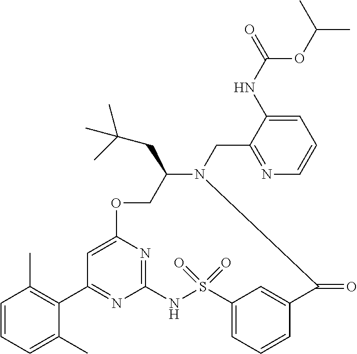 [SMILES: Cc1cccc(C)c1-c1cc2nc(n1)NS(=O)(=O)c1cccc(c1)C(=O)N(Cc1ncccc1NC(=O)OC(C)C)[C@H](CC(C)(C)C)CO2] | | | | | +++ | +++ |
| 391 | 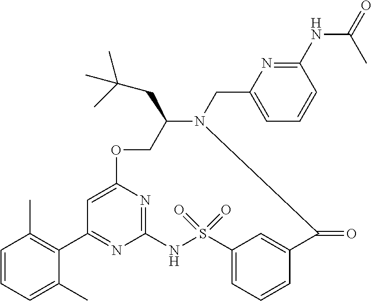 [SMILES: CC(=O)Nc1cccc(CN2C(=O)c3cccc(c3)S(=O)(=O)Nc3nc(cc(-c4c(C)cccc4C)n3)OC[C@H]2CC(C)(C)C)n1] | | | | | +++ | +++ |
| 804 | 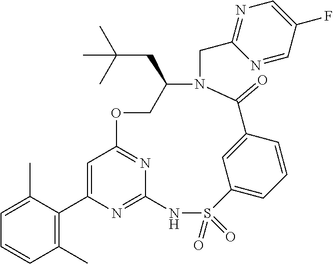 [SMILES: Cc1cccc(C)c1-c1cc2nc(n1)NS(=O)(=O)c1cccc(c1)C(=O)N(Cc1ncc(F)cn1)[C@H](CC(C)(C)C)CO2] | | | | | +++ | +++ |
| 802 | 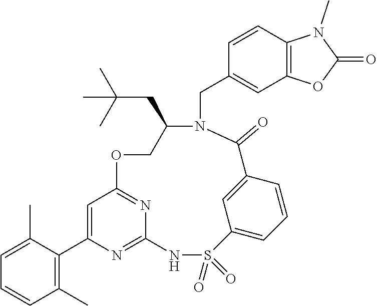 [SMILES: Cc1cccc(C)c1-c1cc2nc(n1)NS(=O)(=O)c1cccc(c1)C(=O)N(Cc1ccc3c(c1)oc(=O)n3C)[C@H](CC(C)(C)C)CO2] | | | | | +++ | +++ |
| 1081 | 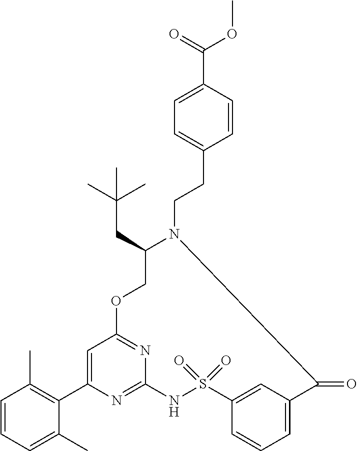 [SMILES: COC(=O)c1ccc(CCN2C(=O)c3cccc(c3)S(=O)(=O)Nc3nc(cc(-c4c(C)cccc4C)n3)OC[C@H]2CC(C)(C)C)cc1] | | | | | +++ | +++ |
| 399 | 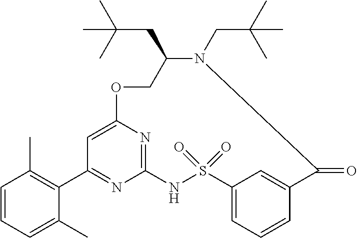 [SMILES: Cc1cccc(C)c1-c1cc2nc(n1)NS(=O)(=O)c1cccc(c1)C(=O)N(CC(C)(C)C)[C@H](CC(C)(C)C)CO2] | | | | | +++ | +++ |
| 807 | 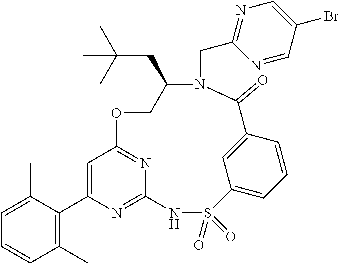 [SMILES: Cc1cccc(C)c1-c1cc2nc(n1)NS(=O)(=O)c1cccc(c1)C(=O)N(Cc1ncc(Br)cn1)[C@H](CC(C)(C)C)CO2] | | | | | +++ | +++ |
| 805 | 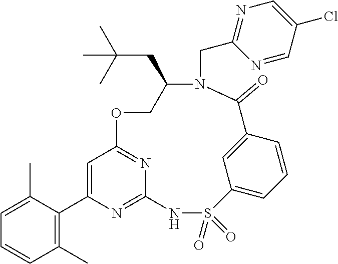 [SMILES: Cc1cccc(C)c1-c1cc2nc(n1)NS(=O)(=O)c1cccc(c1)C(=O)N(Cc1ncc(Cl)cn1)[C@H](CC(C)(C)C)CO2] | | | | | +++ | +++ |
| 806 | 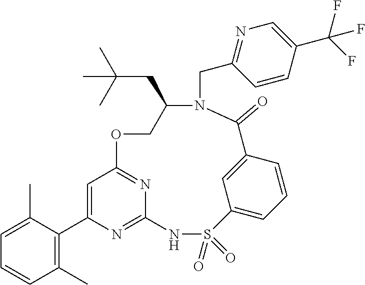 [SMILES: Cc1cccc(C)c1-c1cc2nc(n1)NS(=O)(=O)c1cccc(c1)C(=O)N(Cc1ccc(C(F)(F)F)cn1)[C@H](CC(C)(C)C)CO2] | | | | | +++ | +++ |
| 803 | 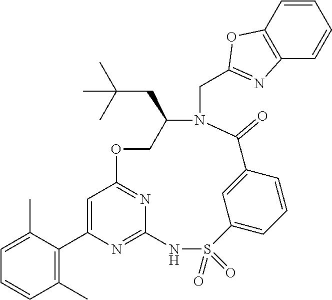 [SMILES: Cc1cccc(C)c1-c1cc2nc(n1)NS(=O)(=O)c1cccc(c1)C(=O)N(Cc1nc3ccccc3o1)[C@H](CC(C)(C)C)CO2] | | | | | +++ | +++ |
| 389 | 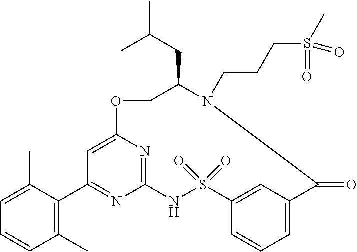 [SMILES: Cc1cccc(C)c1-c1cc2nc(n1)NS(=O)(=O)c1cccc(c1)C(=O)N(CCCS(C)(=O)=O)[C@H](CC(C)C)CO2] | | | | | +++ | +++ |
| 1078 | 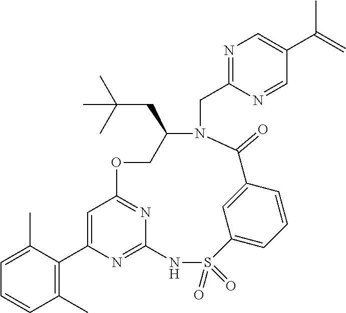 [SMILES: C=C(C)c1cnc(CN2C(=O)c3cccc(c3)S(=O)(=O)Nc3nc(cc(-c4c(C)cccc4C)n3)OC[C@H]2CC(C)(C)C)nc1] | | | | | +++ | +++ |
| 1077 | 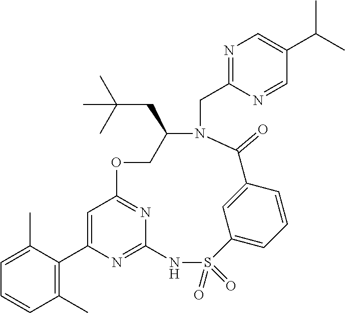 [SMILES: Cc1cccc(C)c1-c1cc2nc(n1)NS(=O)(=O)c1cccc(c1)C(=O)N(Cc1ncc(C(C)C)cn1)[C@H](CC(C)(C)C)CO2] | | | | | +++ | +++ |
| 801 | 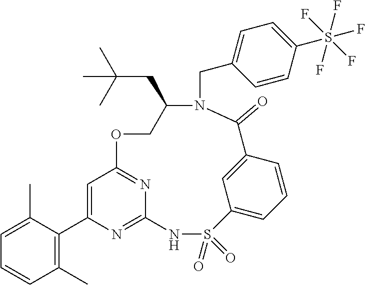 [SMILES: Cc1cccc(C)c1-c1cc2nc(n1)NS(=O)(=O)c1cccc(c1)C(=O)N(Cc1ccc(S(F)(F)(F)(F)F)cc1)[C@H](CC(C)(C)C)CO2] | | | | | +++ | +++ |
| 125 | 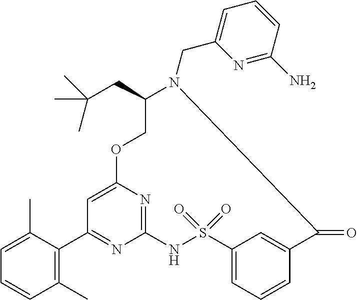 [SMILES: Cc1cccc(C)c1-c1cc2nc(n1)NS(=O)(=O)c1cccc(c1)C(=O)N(Cc1cccc(N)n1)[C@H](CC(C)(C)C)CO2] | | | | | +++ | +++ |
| 388 | 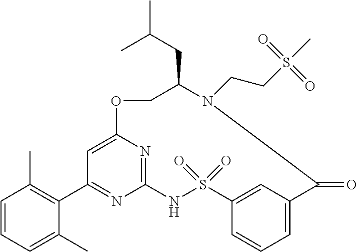 [SMILES: Cc1cccc(C)c1-c1cc2nc(n1)NS(=O)(=O)c1cccc(c1)C(=O)N(CCS(C)(=O)=O)[C@H](CC(C)C)CO2] | | | | | +++ | +++ |
| 799 | 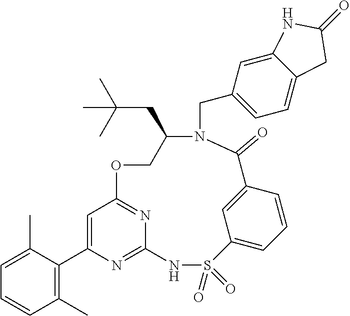 [SMILES: Cc1cccc(C)c1-c1cc2nc(n1)NS(=O)(=O)c1cccc(c1)C(=O)N(Cc1ccc3c(c1)NC(=O)C3)[C@H](CC(C)(C)C)CO2] | | | | | +++ | +++ |
| 798 | 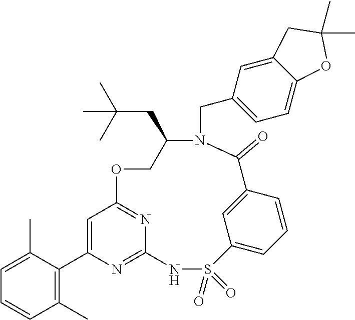 [SMILES: Cc1cccc(C)c1-c1cc2nc(n1)NS(=O)(=O)c1cccc(c1)C(=O)N(Cc1ccc3c(c1)CC(C)(C)O3)[C@H](CC(C)(C)C)CO2] | | | | | +++ | +++ |
| 797 | 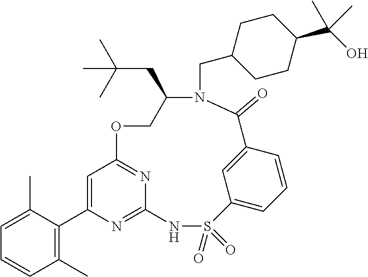 | | | | | +++ | +++ |
| 796 | 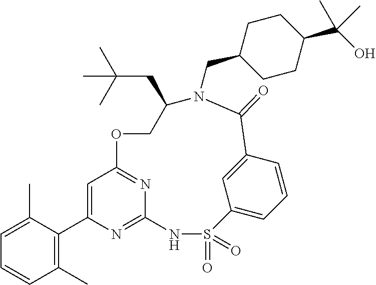 [SMILES: Cc1cccc(C)c1-c1cc2nc(n1)NS(=O)(=O)c1cccc(c1)C(=O)N(C[C@H]1CC[C@@H](C(C)(C)O)CC1)[C@H](CC(C)(C)C)CO2] | | | | | +++ | +++ |
| 108 | 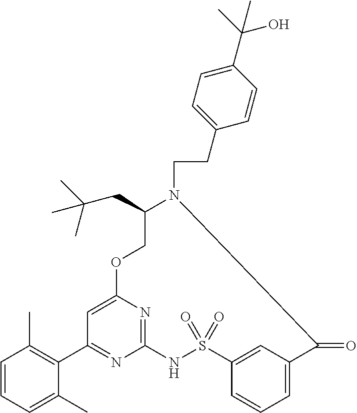 [SMILES: Cc1cccc(C)c1-c1cc2nc(n1)NS(=O)(=O)c1cccc(c1)C(=O)N(CCc1ccc(C(C)(C)O)cc1)[C@H](CC(C)(C)C)CO2] | | | | | +++ | +++ |
| 321 | 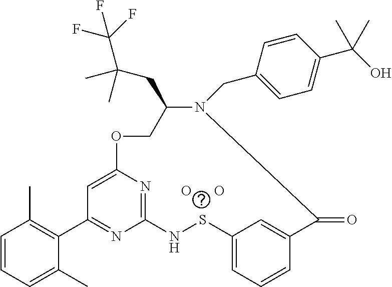 | | | | | +++ | +++ |
| 320 | 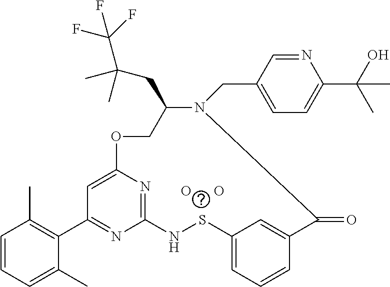 | | | | | +++ | +++ |
| 843 | 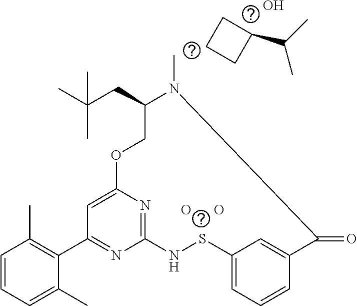 | | | | | +++ | +++ |
| 844 | 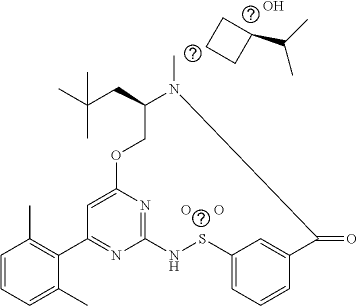 | | | | | +++ | +++ |
| 841 | 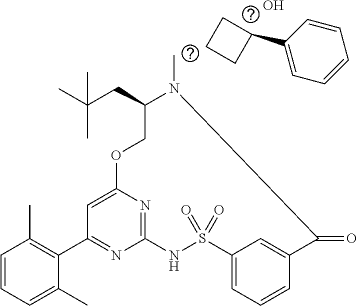 | | | | | +++ | +++ |
| 842 | 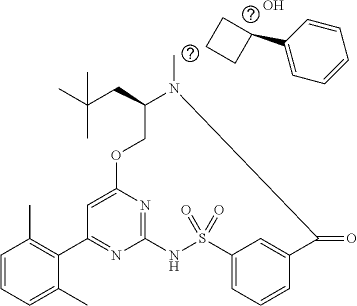 | | | | | +++ | +++ |
| 839 | 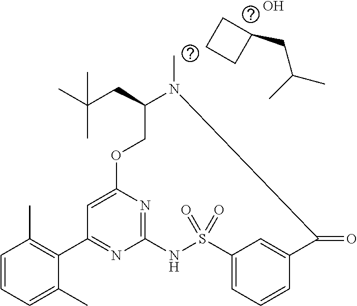 | | | | | +++ | +++ |
| 840 | 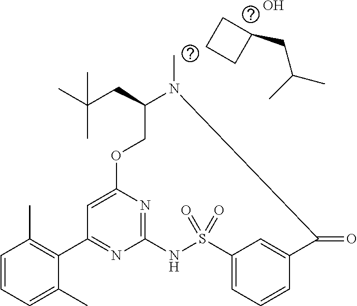 | | | | | +++ | +++ |
| 67 | 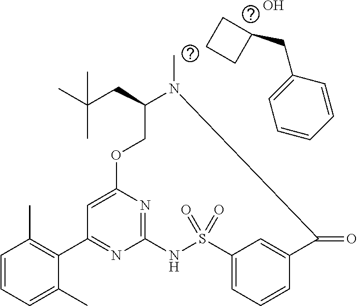 | | | | | +++ | +++ |
| 68 | 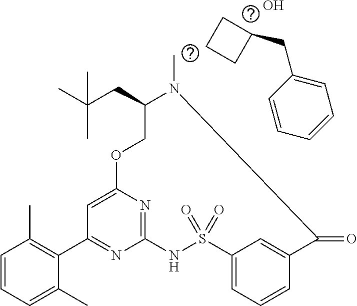 | | | | | +++ | +++ |
| 319 | 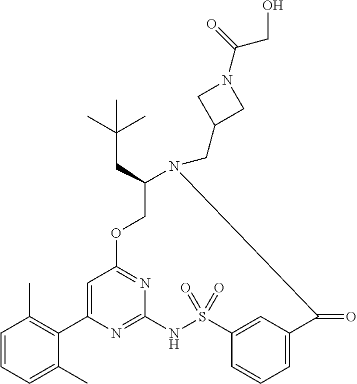 [SMILES: Cc1cccc(C)c1-c1cc2nc(n1)NS(=O)(=O)c1cccc(c1)C(=O)N(CC1CN(C(=O)CO)C1)[C@H](CC(C)(C)C)CO2] | | | | | + | + |
| 793 | 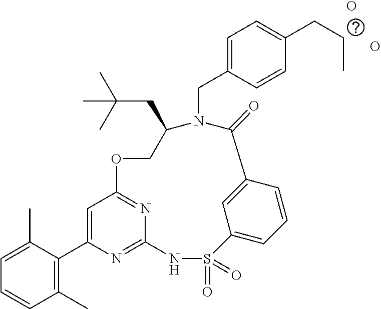 | | | | | +++ | +++ |
| 792 | 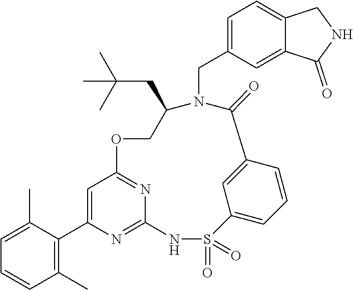 [SMILES: Cc1cccc(C)c1-c1cc2nc(n1)NS(=O)(=O)c1cccc(c1)C(=O)N(Cc1ccc3c(c1)C(=O)NC3)[C@H](CC(C)(C)C)CO2] | | | | | +++ | +++ |
| 800 | 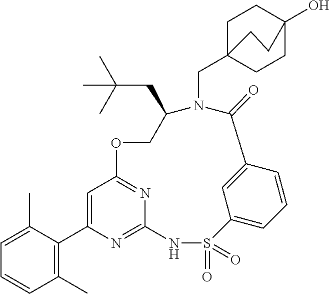 [SMILES: Cc1cccc(C)c1-c1cc2nc(n1)NS(=O)(=O)c1cccc(c1)C(=O)N(CC13CCC(O)(CC1)CC3)[C@H](CC(C)(C)C)CO2] | | | | | +++ | +++ |
| 791 | 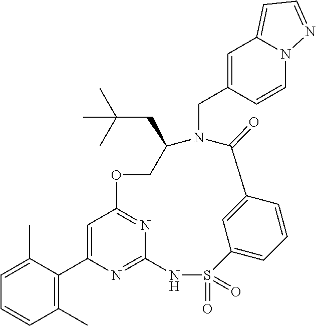 [SMILES: Cc1cccc(C)c1-c1cc2nc(n1)NS(=O)(=O)c1cccc(c1)C(=O)N(Cc1ccn3nccc3c1)[C@H](CC(C)(C)C)CO2] | | | | | +++ | +++ |
| 790 | 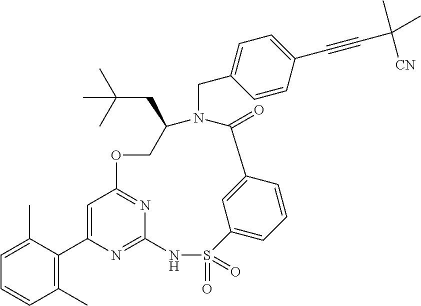 [SMILES: Cc1cccc(C)c1-c1cc2nc(n1)NS(=O)(=O)c1cccc(c1)C(=O)N(Cc1ccc(C#CC(C)(C)C#N)cc1)[C@H](CC(C)(C)C)CO2] | | | | | +++ | +++ |
| 318 | 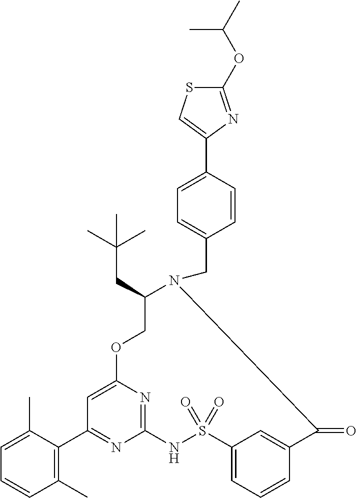 [SMILES: Cc1cccc(C)c1-c1cc2nc(n1)NS(=O)(=O)c1cccc(c1)C(=O)N(Cc1ccc(-c3csc(OC(C)C)n3)cc1)[C@H](CC(C)(C)C)CO2] | | | | | +++ | +++ |
| 317 | 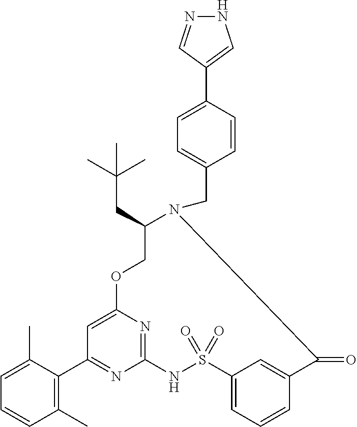 [SMILES: Cc1cccc(C)c1-c1cc2nc(n1)NS(=O)(=O)c1cccc(c1)C(=O)N(Cc1ccc(-c3cn[nH]c3)cc1)[C@H](CC(C)(C)C)CO2] | | | | | +++ | +++ |
| 387 | 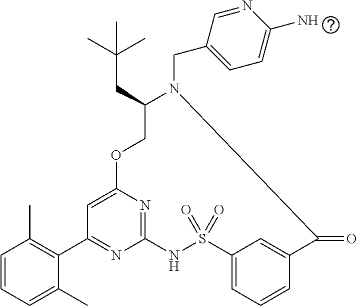 [SMILES: Cc1cccc(C)c1-c1cc2nc(n1)NS(=O)(=O)c1cccc(c1)C(=O)N(Cc1ccc(N)nc1)[C@H](CC(C)(C)C)CO2] | | | | | +++ | +++ |
| 61 | 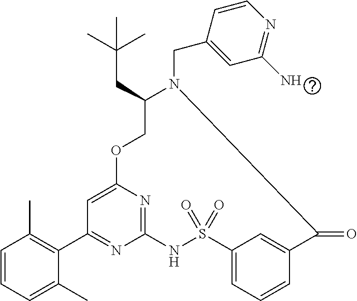 [SMILES: Cc1cccc(C)c1-c1cc2nc(n1)NS(=O)(=O)c1cccc(c1)C(=O)N(Cc1ccnc(N)c1)[C@H](CC(C)(C)C)CO2] | | | | | +++ | +++ |
| 385 | 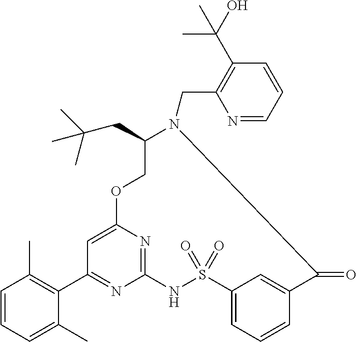 [SMILES: Cc1cccc(C)c1-c1cc2nc(n1)NS(=O)(=O)c1cccc(c1)C(=O)N(Cc1ncccc1C(C)(C)O)[C@H](CC(C)(C)C)CO2] | | | | | +++ | +++ |
| 48 | 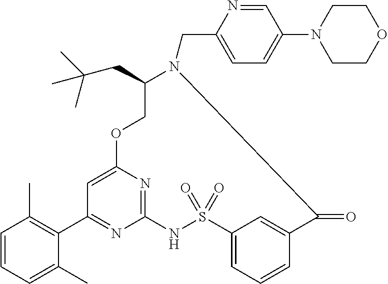 [SMILES: Cc1cccc(C)c1-c1cc2nc(n1)NS(=O)(=O)c1cccc(c1)C(=O)N(Cc1ccc(N3CCOCC3)cn1)[C@H](CC(C)(C)C)CO2] | | | | | +++ | +++ |
| 386 | 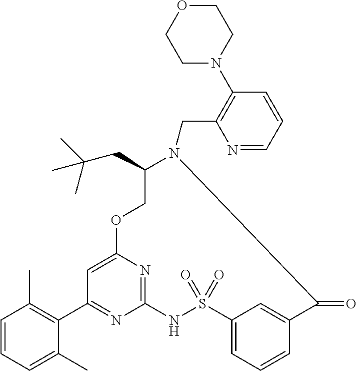 [SMILES: Cc1cccc(C)c1-c1cc2nc(n1)NS(=O)(=O)c1cccc(c1)C(=O)N(Cc1ncccc1N1CCOCC1)[C@H](CC(C)(C)C)CO2] | | | | | +++ | +++ |
| 69 | 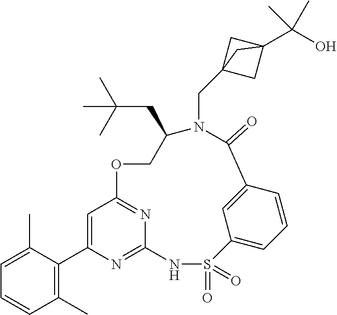 [SMILES: Cc1cccc(C)c1-c1cc2nc(n1)NS(=O)(=O)c1cccc(c1)C(=O)N(CC13CC(C(C)(C)O)(C1)C3)[C@H](CC(C)(C)C)CO2] | | | | | +++ | +++ |
| 789 | 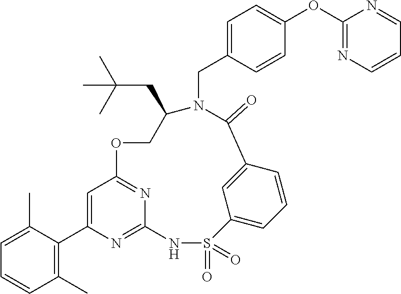 [SMILES: Cc1cccc(C)c1-c1cc2nc(n1)NS(=O)(=O)c1cccc(c1)C(=O)N(Cc1ccc(Oc3ncccn3)cc1)[C@H](CC(C)(C)C)CO2] | | | | | +++ | +++ |
| 788 | 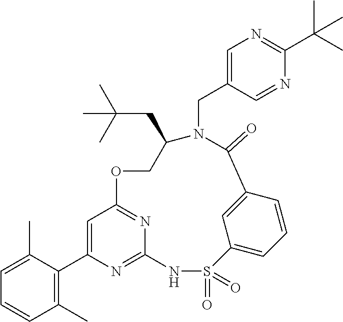 [SMILES: Cc1cccc(C)c1-c1cc2nc(n1)NS(=O)(=O)c1cccc(c1)C(=O)N(Cc1cnc(C(C)(C)C)nc1)[C@H](CC(C)(C)C)CO2] | | | | | +++ | +++ |
| 316 | 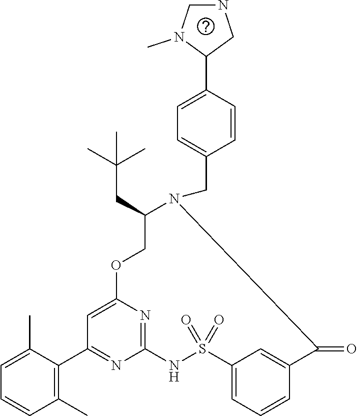 [SMILES: Cc1cccc(C)c1-c1cc2nc(n1)NS(=O)(=O)c1cccc(c1)C(=O)N(Cc1ccc(C3CNCN3C)cc1)[C@H](CC(C)(C)C)CO2] | | | | | +++ | +++ |
| 1190 | 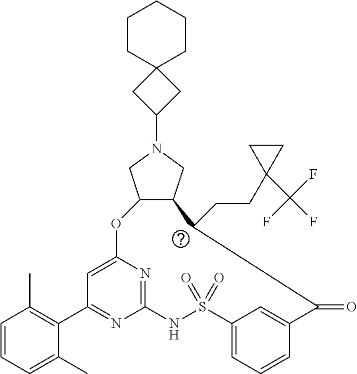 [SMILES: Cc1cccc(C)c1-c1cc2nc(n1)NS(=O)(=O)c1cccc(c1)C(=O)C(CCC1(C(F)(F)F)CC1)[C@@H]1CN(C3CC4(CCCCC4)C3)CC1O2] | | | | | +++ | ++ |
| 1191 | 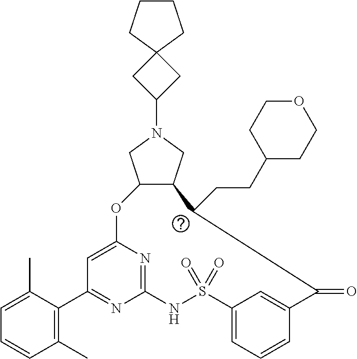 [SMILES: Cc1cccc(C)c1-c1cc2nc(n1)NS(=O)(=O)c1cccc(c1)C(=O)C(CCC1CCOCC1)[C@@H]1CN(C3CC4(CCCC4)C3)CC1O2] | | | | | +++ | +++ |
| 1192 | 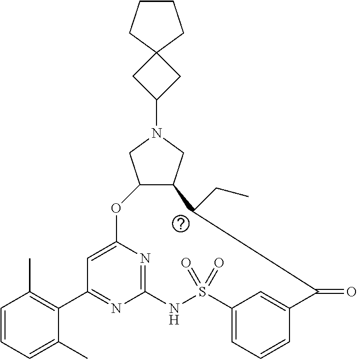 [SMILES: CCC1C(=O)c2cccc(c2)S(=O)(=O)Nc2nc(cc(-c3c(C)cccc3C)n2)OC2CN(C3CC4(CCCC4)C3)C[C@H]21] | | | +++ | +++ | +++ | ++ |
| 1188 | 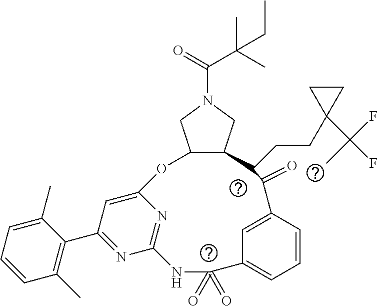 | | | +++ | +++ | +++ | ++ |
| 1189 | 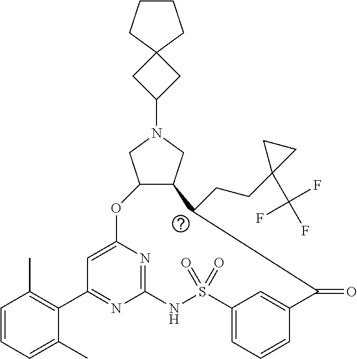 [SMILES: Cc1cccc(C)c1-c1cc2nc(n1)NS(=O)(=O)c1cccc(c1)C(=O)C(CCC1(C(F)(F)F)CC1)[C@@H]1CN(C3CC4(CCCC4)C3)CC1O2] | | | +++ | +++ | +++ | ++ |

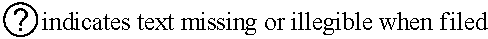

7 Biological Activity Data

Table 10 represents CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” 00 Activity: +++ is >60%; ++ is 30-60%; + is <300%).

TABLE 10

| | | HBE | HBE Max | HBE |
|---|---|---|---|---|
| Cmpd | | EC₅₀ | activity | activity at |
| Number | Structure | (μM) | (%) | 10 (μM) (%) |
| 143 | 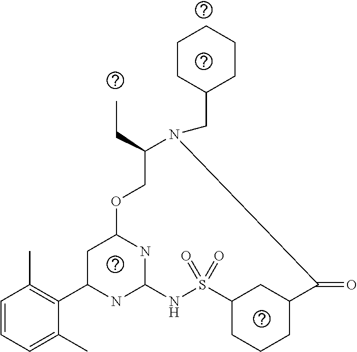 [SMILES: CC[C@@H]1COC2CC(c3c(C)cccc3C)NC(N2)NS(=O)(=O)C2CCCC(C2)C(=O)N1CC1CCCCC1] | +++ | +++ | |
| 63 | 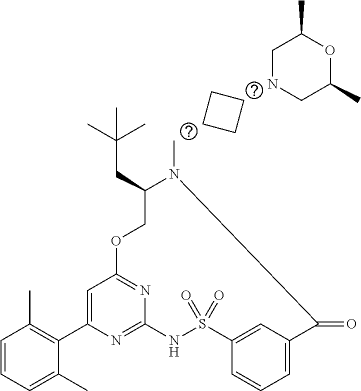 | +++ | +++ | |
| 64 | 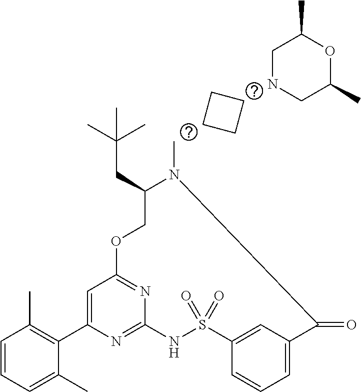 | +++ | +++ | |
| 315 | 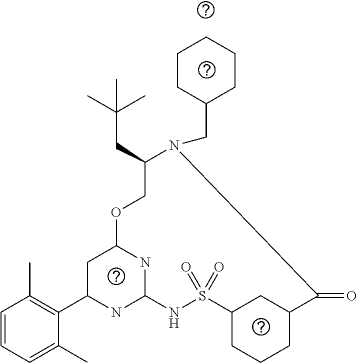 [SMILES: Cc1cccc(C)c1C1CC2NC(N1)NS(=O)(=O)C1CCCC(C1)C(=O)N(CC1CCCCC1)[C@H](CC(C)(C)C)CO2] | +++ | +++ | |
| 314 | 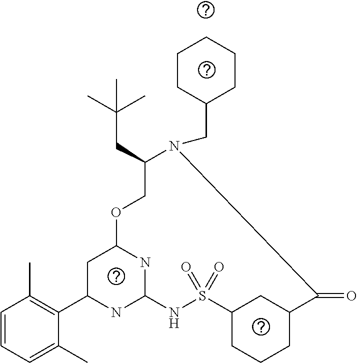 [SMILES: Cc1cccc(C)c1C1CC2NC(N1)NS(=O)(=O)C1CCCC(C1)C(=O)N(CC1CCCCC1)[C@H](CC(C)(C)C)CO2] | +++ | +++ | |
| 787 | 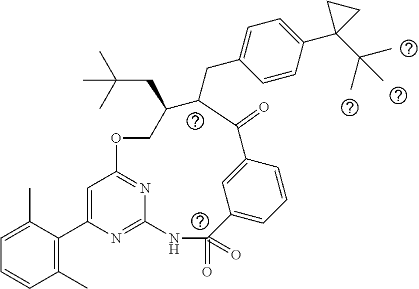 | +++ | +++ | |
| 794 | 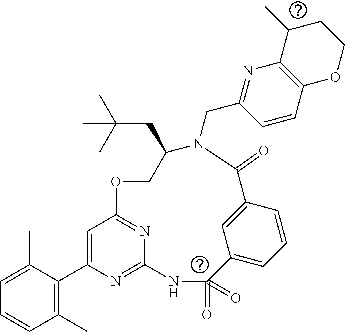 | +++ | +++ | |
| 786 | 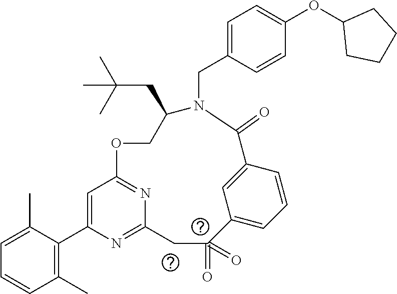 | +++ | +++ | |
| 785 | 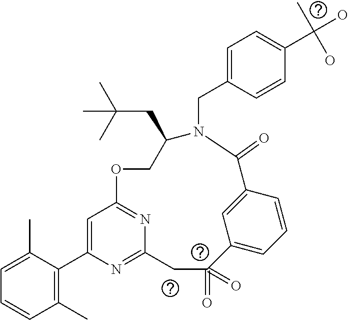 | +++ | +++ | |
| 384 | 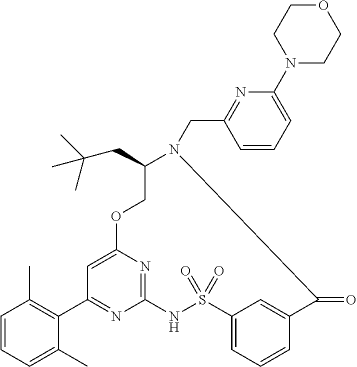 [SMILES: Cc1cccc(C)c1-c1cc2nc(n1)NS(=O)(=O)c1cccc(c1)C(=O)N(Cc1cccc(N3CCOCC3)n1)[C@H](CC(C)(C)C)CO2] | +++ | +++ | |
| 313 | 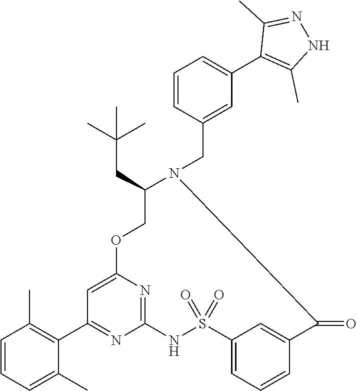 [SMILES: Cc1cccc(C)c1-c1cc2nc(n1)NS(=O)(=O)c1cccc(c1)C(=O)N(Cc1cccc(-c3c(C)n[nH]c3C)c1)[C@H](CC(C)(C)C)CO2] | +++ | +++ | |
| 312 | 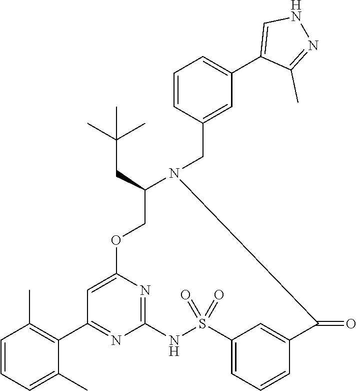 [SMILES: Cc1cccc(C)c1-c1cc2nc(n1)NS(=O)(=O)c1cccc(c1)C(=O)N(Cc1cccc(-c3c[nH]nc3C)c1)[C@H](CC(C)(C)C)CO2] | | | +++ |
| 311 | 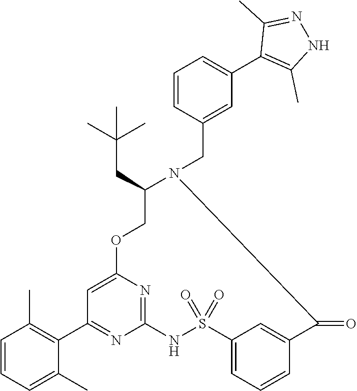 [SMILES: Cc1cccc(C)c1-c1cc2nc(n1)NS(=O)(=O)c1cccc(c1)C(=O)N(Cc1cccc(-c3c(C)n[nH]c3C)c1)[C@H](CC(C)(C)C)CO2] | +++ | +++ | |
| 310 | 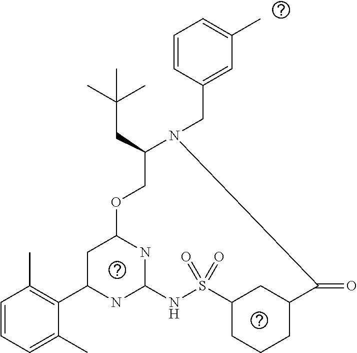 [SMILES: Cc1cccc(CN2C(=O)C3CCCC(C3)S(=O)(=O)NC3NC(CC(c4c(C)cccc4C)N3)OC[C@H]2CC(C)(C)C)c1] | +++ | +++ | |
| 309 | 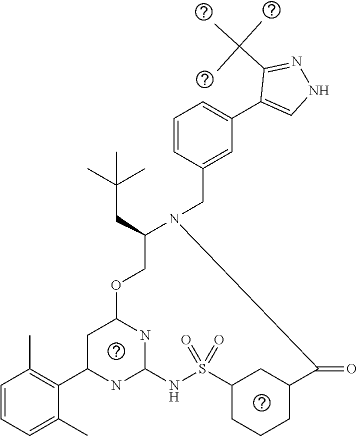 [SMILES: Cc1cccc(C)c1C1CC2NC(N1)NS(=O)(=O)C1CCCC(C1)C(=O)N(Cc1cccc(-c3c[nH]nc3C(C)(C)C)c1)[C@H](CC(C)(C)C)CO2] | +++ | +++ | |
| 308 | 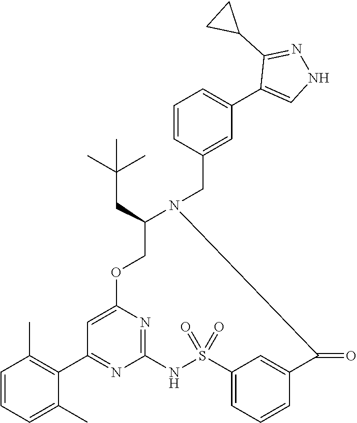 [SMILES: Cc1cccc(C)c1-c1cc2nc(n1)NS(=O)(=O)c1cccc(c1)C(=O)N(Cc1cccc(-c3c[nH]nc3C3CC3)c1)[C@H](CC(C)(C)C)CO2] | +++ | +++ | |
| 784 | 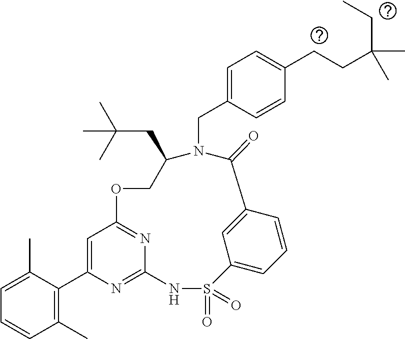 [SMILES: CCC(C)(C)CCc1ccc(CN2C(=O)c3cccc(c3)S(=O)(=O)Nc3nc(cc(-c4c(C)cccc4C)n3)OC[C@H]2CC(C)(C)C)cc1] | +++ | +++ | |
| 376 | 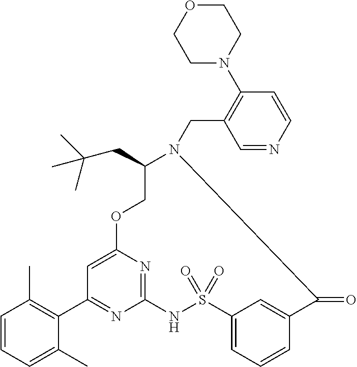 [SMILES: Cc1cccc(C)c1-c1cc2nc(n1)NS(=O)(=O)c1cccc(c1)C(=O)N(Cc1cnccc1N1CCOCC1)[C@H](CC(C)(C)C)CO2] | +++ | +++ | |
| 375 | 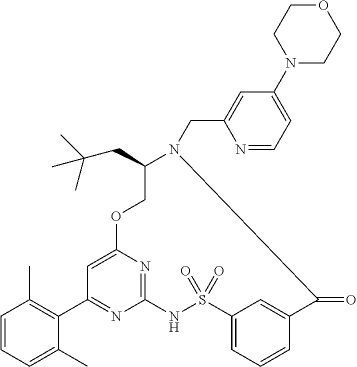 [SMILES: Cc1cccc(C)c1-c1cc2nc(n1)NS(=O)(=O)c1cccc(c1)C(=O)N(Cc1cc(N3CCOCC3)ccn1)[C@H](CC(C)(C)C)CO2] | +++ | +++ | |
| 374 | 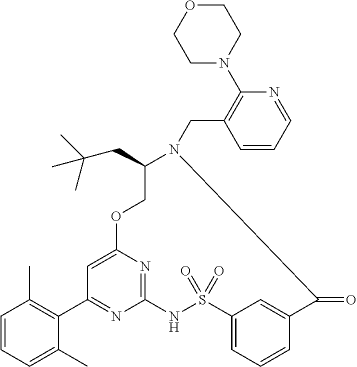 [SMILES: Cc1cccc(C)c1-c1cc2nc(n1)NS(=O)(=O)c1cccc(c1)C(=O)N(Cc1cccnc1N1CCOCC1)[C@H](CC(C)(C)C)CO2] | +++ | +++ | |
| 54 | 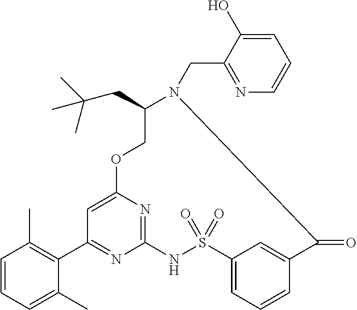 [SMILES: Cc1cccc(C)c1-c1cc2nc(n1)NS(=O)(=O)c1cccc(c1)C(=O)N(Cc1ncccc1O)[C@H](CC(C)(C)C)CO2] | +++ | +++ | |
| 382 | 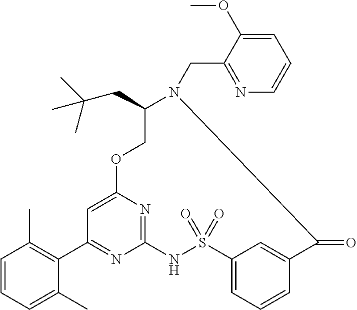 [SMILES: COc1cccnc1CN1C(=O)c2cccc(c2)S(=O)(=O)Nc2nc(cc(-c3c(C)cccc3C)n2)OC[C@H]1CC(C)(C)C] | +++ | +++ | |
| 65 | 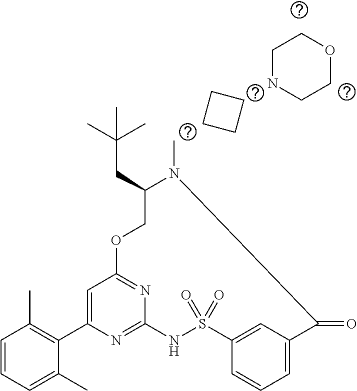 | | | +++ |
| 66 | 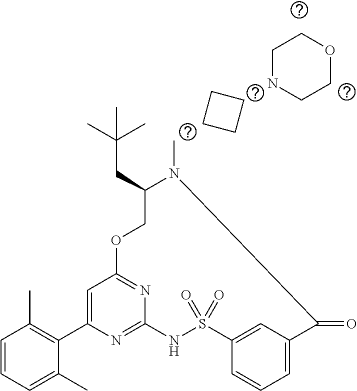 | | | +++ |
| 915 | 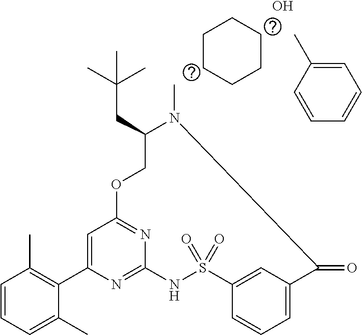 | +++ | +++ | |
| 916 | 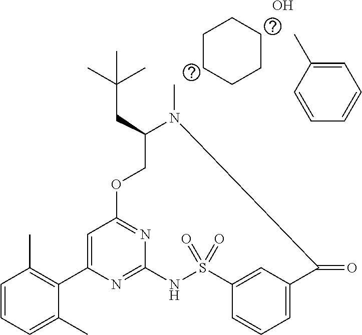 | +++ | +++ | |
| 919 | 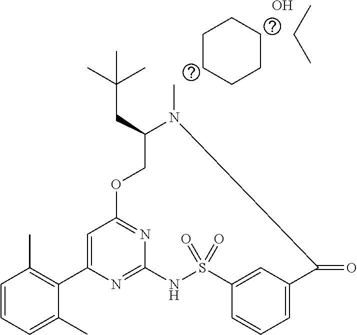 | +++ | +++ | |
| 920 | 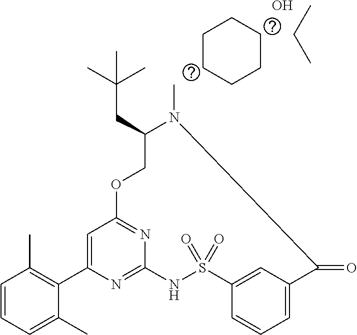 | +++ | +++ | |
| 917 | 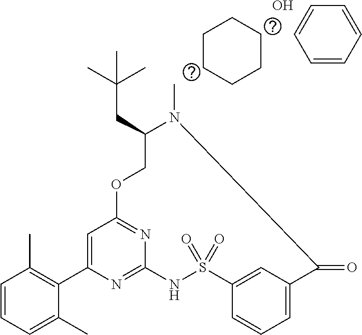 | +++ | +++ | |
| 918 | 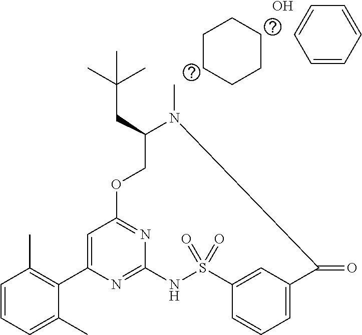 | +++ | +++ | |
| 1076 | 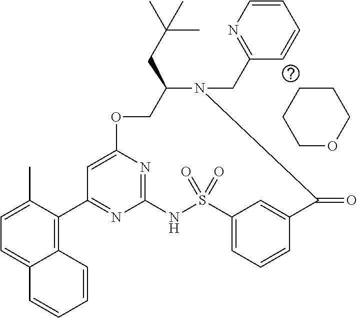 | +++ | +++ | |
| 147 | 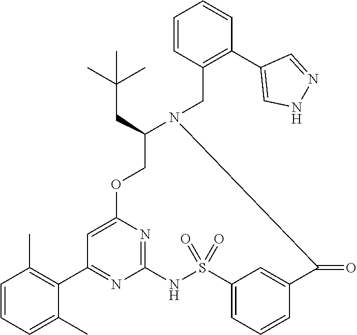 [SMILES: Cc1cccc(C)c1-c1cc2nc(n1)NS(=O)(=O)c1cccc(c1)C(=O)N(Cc1ccccc1-c1cn[nH]c1)[C@H](CC(C)(C)C)CO2] | +++ | +++ | |
| 307 | 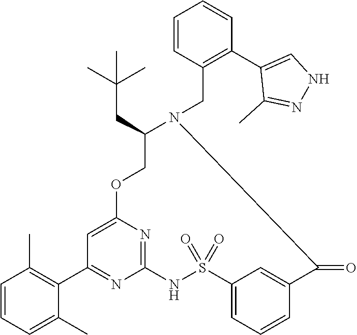 [SMILES: Cc1cccc(C)c1-c1cc2nc(n1)NS(=O)(=O)c1cccc(c1)C(=O)N(Cc1ccccc1-c1c[nH]nc1C)[C@H](CC(C)(C)C)CO2] | | | +++ |
| 306 | 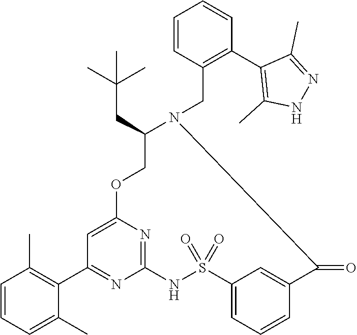 [SMILES: Cc1cccc(C)c1-c1cc2nc(n1)NS(=O)(=O)c1cccc(c1)C(=O)N(Cc1ccccc1-c1c(C)n[nH]c1C)[C@H](CC(C)(C)C)CO2] | +++ | +++ | |
| 305 | 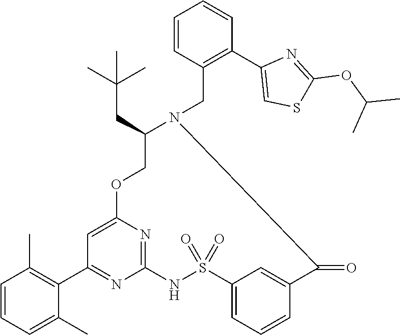 [SMILES: Cc1cccc(C)c1-c1cc2nc(n1)NS(=O)(=O)c1cccc(c1)C(=O)N(Cc1ccccc1-c1csc(OC(C)C)n1)[C@H](CC(C)(C)C)CO2] | | | +++ |
| 304 | 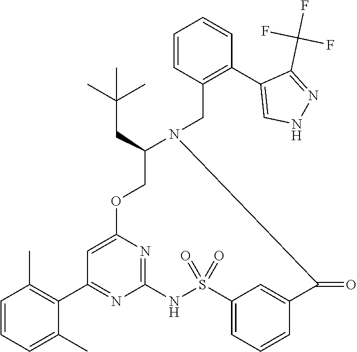 [SMILES: Cc1cccc(C)c1-c1cc2nc(n1)NS(=O)(=O)c1cccc(c1)C(=O)N(Cc1ccccc1-c1c[nH]nc1C(F)(F)F)[C@H](CC(C)(C)C)CO2] | | | +++ |
| 303 | 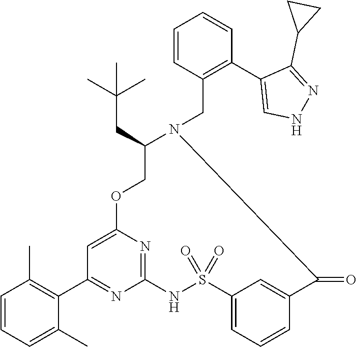 [SMILES: Cc1cccc(C)c1-c1cc2nc(n1)NS(=O)(=O)c1cccc(c1)C(=O)N(Cc1ccccc1-c1c[nH]nc1C1CC1)[C@H](CC(C)(C)C)CO2] | +++ | +++ | |
| 381 | 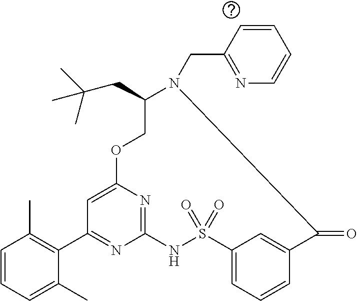 [SMILES: Cc1cccc(C)c1-c1cc2nc(n1)NS(=O)(=O)c1cccc(c1)C(=O)N(Cc1ccccn1)[C@H](CC(C)(C)C)CO2] | +++ | +++ | |
| 380 | 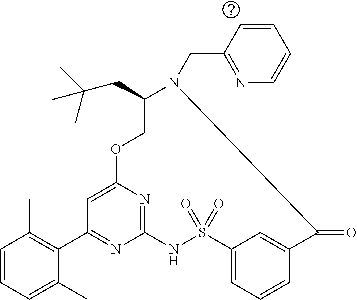 [SMILES: Cc1cccc(C)c1-c1cc2nc(n1)NS(=O)(=O)c1cccc(c1)C(=O)N(Cc1ccccn1)[C@H](CC(C)(C)C)CO2] | +++ | +++ | |
| 379 | 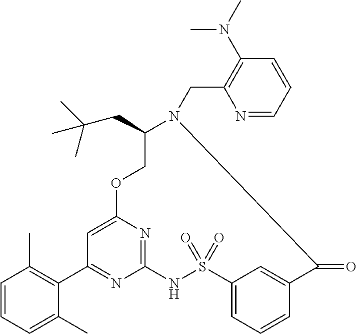 [SMILES: Cc1cccc(C)c1-c1cc2nc(n1)NS(=O)(=O)c1cccc(c1)C(=O)N(Cc1ncccc1N(C)C)[C@H](CC(C)(C)C)CO2] | +++ | +++ | |
| 378 | 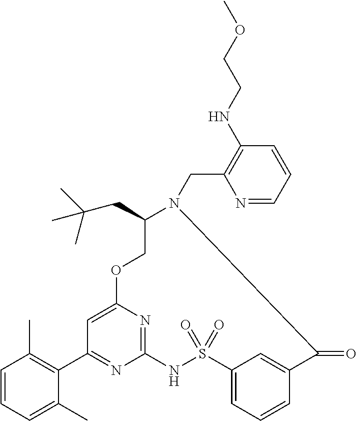 [SMILES: COCCNc1cccnc1CN1C(=O)c2cccc(c2)S(=O)(=O)Nc2nc(cc(-c3c(C)cccc3C)n2)OC[C@H]1CC(C)(C)C] | +++ | +++ | |
| 377 | 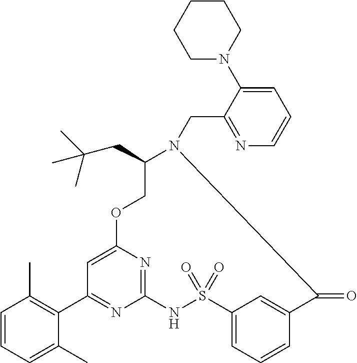 [SMILES: Cc1cccc(C)c1-c1cc2nc(n1)NS(=O)(=O)c1cccc(c1)C(=O)N(Cc1ncccc1N1CCCCC1)[C@H](CC(C)(C)C)CO2] | +++ | +++ | |
| 383 | 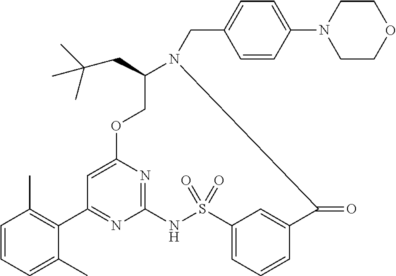 [SMILES: Cc1cccc(C)c1-c1cc2nc(n1)NS(=O)(=O)c1cccc(c1)C(=O)N(Cc1ccc(N3CCOCC3)cc1)[C@H](CC(C)(C)C)CO2] | +++ | +++ | |
| 1075 | 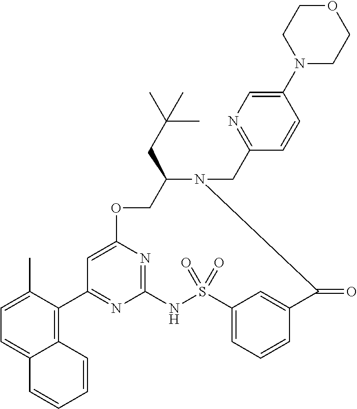 [SMILES: Cc1ccc2ccccc2c1-c1cc2nc(n1)NS(=O)(=O)c1cccc(c1)C(=O)N(Cc1ccc(N3CCOCC3)cn1)[C@H](CC(C)(C)C)CO2] | +++ | +++ | |
| 144 | 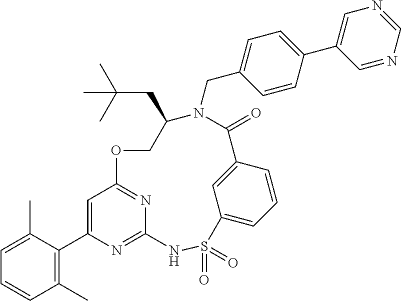 [SMILES: Cc1cccc(C)c1-c1cc2nc(n1)NS(=O)(=O)c1cccc(c1)C(=O)N(Cc1ccc(-c3cncnc3)cc1)[C@H](CC(C)(C)C)CO2] | +++ | +++ | |
| 145 | 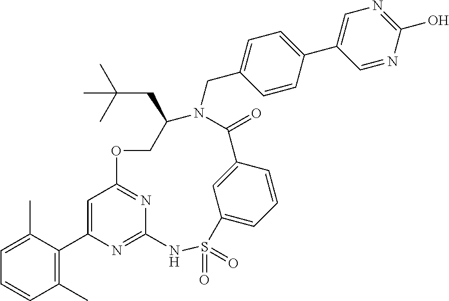 [SMILES: Cc1cccc(C)c1-c1cc2nc(n1)NS(=O)(=O)c1cccc(c1)C(=O)N(Cc1ccc(-c3cnc(O)nc3)cc1)[C@H](CC(C)(C)C)CO2] | ++ | +++ | |
| 782 | 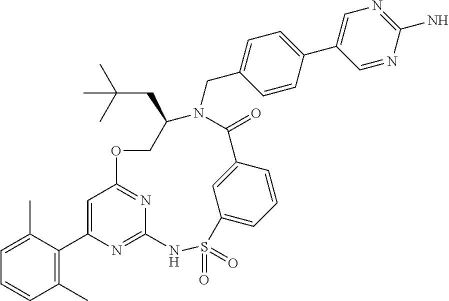 [SMILES: Cc1cccc(C)c1-c1cc2nc(n1)NS(=O)(=O)c1cccc(c1)C(=O)N(Cc1ccc(-c3cnc(N)nc3)cc1)[C@H](CC(C)(C)C)CO2] | +++ | +++ | |
| 783 | 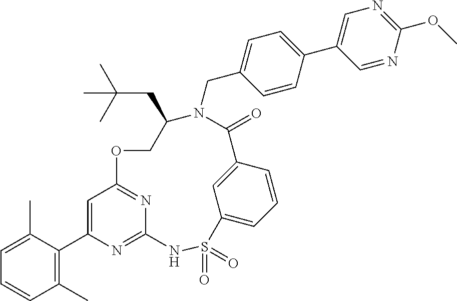 [SMILES: COc1ncc(-c2ccc(CN3C(=O)c4cccc(c4)S(=O)(=O)Nc4nc(cc(-c5c(C)cccc5C)n4)OC[C@H]3CC(C)(C)C)cc2)cn1] | +++ | +++ | |
| 780 | 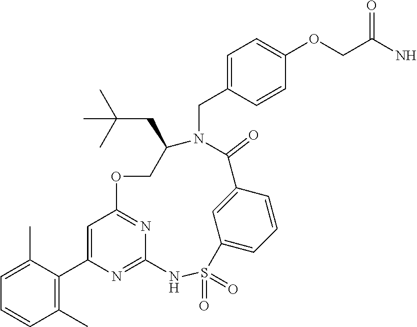 [SMILES: Cc1cccc(C)c1-c1cc2nc(n1)NS(=O)(=O)c1cccc(c1)C(=O)N(Cc1ccc(OCC(N)=O)cc1)[C@H](CC(C)(C)C)CO2] | +++ | +++ | |
| 778 | 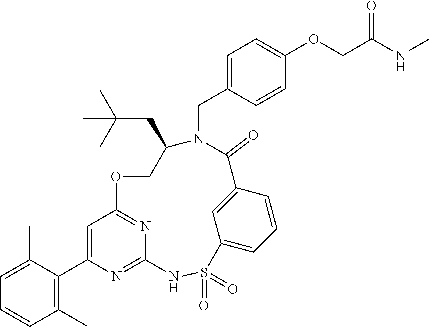 [SMILES: CNC(=O)COc1ccc(CN2C(=O)c3cccc(c3)S(=O)(=O)Nc3nc(cc(-c4c(C)cccc4C)n3)OC[C@H]2CC(C)(C)C)cc1] | +++ | +++ | |
| 60 | 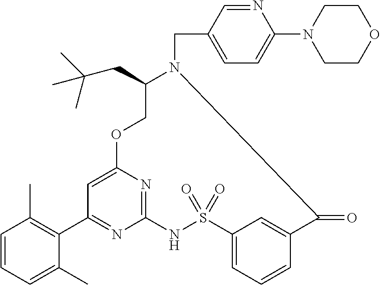 [SMILES: Cc1cccc(C)c1-c1cc2nc(n1)NS(=O)(=O)c1cccc(c1)C(=O)N(Cc1ccc(N3CCOCC3)nc1)[C@H](CC(C)(C)C)CO2] | +++ | +++ | |
| 779 | 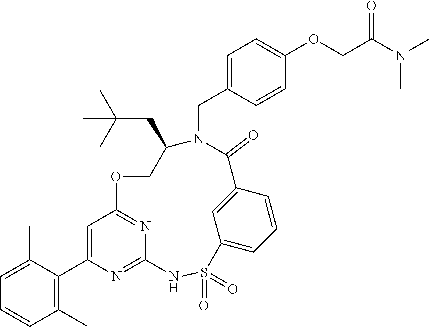 [SMILES: Cc1cccc(C)c1-c1cc2nc(n1)NS(=O)(=O)c1cccc(c1)C(=O)N(Cc1ccc(OCC(=O)N(C)C)cc1)[C@H](CC(C)(C)C)CO2] | +++ | +++ | |
| 777 | 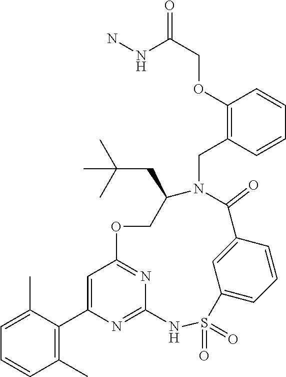 [SMILES: Cc1cccc(C)c1-c1cc2nc(n1)NS(=O)(=O)c1cccc(c1)C(=O)N(Cc1ccccc1OCC(=O)NN)[C@H](CC(C)(C)C)CO2] | | | +++ |
| 775 | 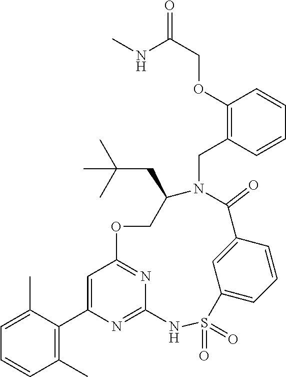 [SMILES: CNC(=O)COc1ccccc1CN1C(=O)c2cccc(c2)S(=O)(=O)Nc2nc(cc(-c3c(C)cccc3C)n2)OC[C@H]1CC(C)(C)C] | | | +++ |
| 776 | 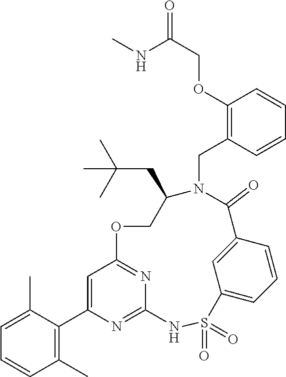 [SMILES: CNC(=O)COc1ccccc1CN1C(=O)c2cccc(c2)S(=O)(=O)Nc2nc(cc(-c3c(C)cccc3C)n2)OC[C@H]1CC(C)(C)C] | +++ | +++ | +++ |
| 53 | 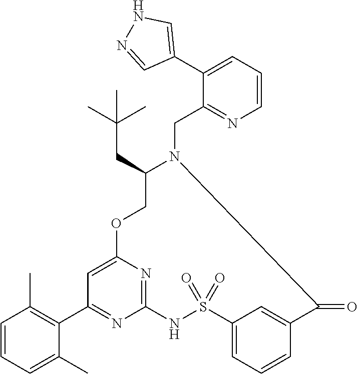 [SMILES: Cc1cccc(C)c1-c1cc2nc(n1)NS(=O)(=O)c1cccc(c1)C(=O)N(Cc1ncccc1-c1cn[nH]c1)[C@H](CC(C)(C)C)CO2] | +++ | +++ | +++ |
| 301 | 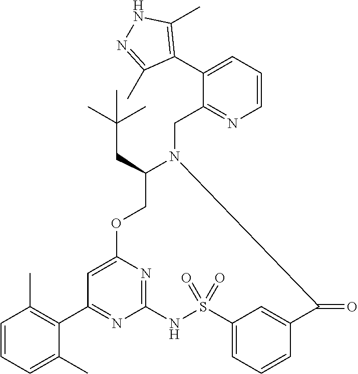 [SMILES: Cc1cccc(C)c1-c1cc2nc(n1)NS(=O)(=O)c1cccc(c1)C(=O)N(Cc1ncccc1-c1c(C)n[nH]c1C)[C@H](CC(C)(C)C)CO2] | +++ | +++ | |
| 300 | 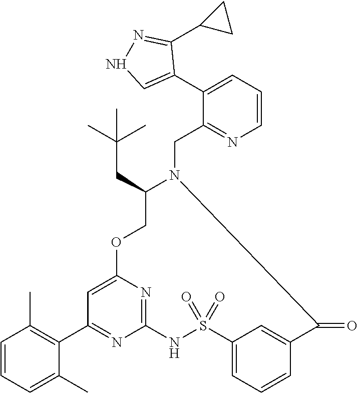 [SMILES: Cc1cccc(C)c1-c1cc2nc(n1)NS(=O)(=O)c1cccc(c1)C(=O)N(Cc1ncccc1-c1c[nH]nc1C1CC1)[C@H](CC(C)(C)C)CO2] | | | +++ |
| 299 | 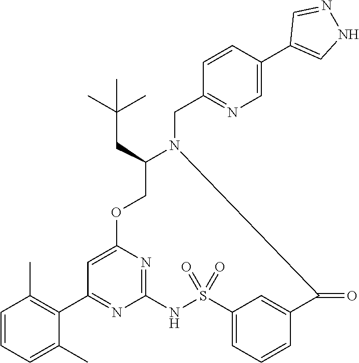 [SMILES: Cc1cccc(C)c1-c1cc2nc(n1)NS(=O)(=O)c1cccc(c1)C(=O)N(Cc1ccc(-c3cn[nH]c3)cn1)[C@H](CC(C)(C)C)CO2] | +++ | +++ | |
| 298 | 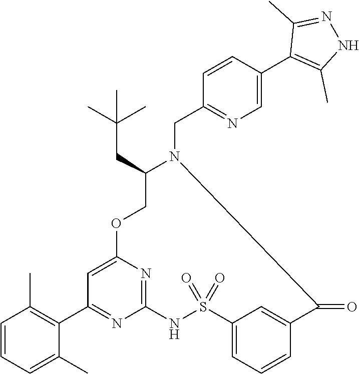 [SMILES: Cc1cccc(C)c1-c1cc2nc(n1)NS(=O)(=O)c1cccc(c1)C(=O)N(Cc1ccc(-c3c(C)n[nH]c3C)cn1)[C@H](CC(C)(C)C)CO2] | ++ | +++ | |
| 297 | 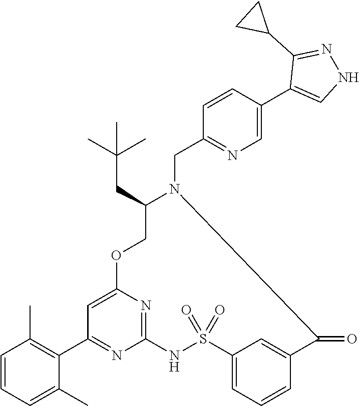 [SMILES: Cc1cccc(C)c1-c1cc2nc(n1)NS(=O)(=O)c1cccc(c1)C(=O)N(Cc1ccc(-c3c[nH]nc3C3CC3)cn1)[C@H](CC(C)(C)C)CO2] | +++ | +++ | |
| 302 | 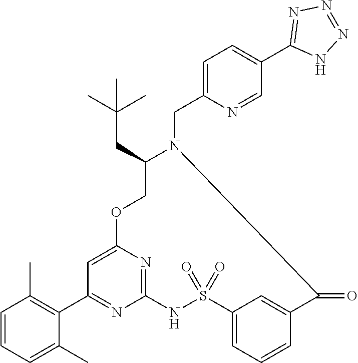 [SMILES: Cc1cccc(C)c1-c1cc2nc(n1)NS(=O)(=O)c1cccc(c1)C(=O)N(Cc1ccc(-c3nnn[nH]3)cn1)[C@H](CC(C)(C)C)CO2] | ND | | |
| 504 | 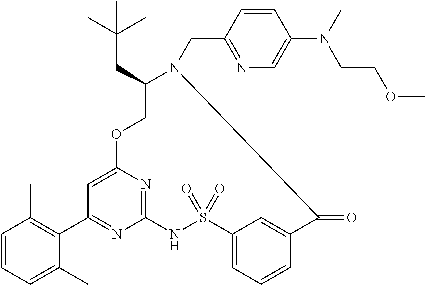 [SMILES: COCCN(C)c1ccc(CN2C(=O)c3cccc(c3)S(=O)(=O)Nc3nc(cc(-c4c(C)cccc4C)n3)OC[C@H]2CC(C)(C)C)nc1] | ++ | +++ | |
| 503 | 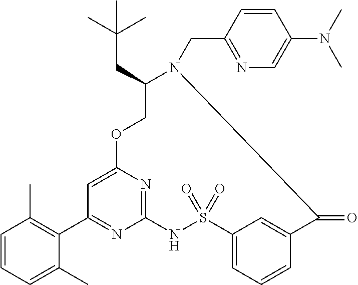 [SMILES: Cc1cccc(C)c1-c1cc2nc(n1)NS(=O)(=O)c1cccc(c1)C(=O)N(Cc1ccc(N(C)C)cn1)[C@H](CC(C)(C)C)CO2] | +++ | +++ | |
| 502 | 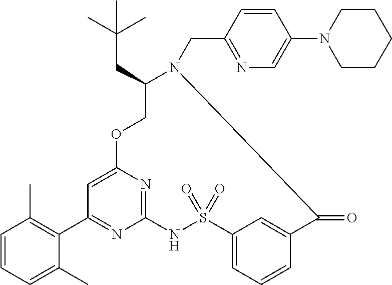 [SMILES: Cc1cccc(C)c1-c1cc2nc(n1)NS(=O)(=O)c1cccc(c1)C(=O)N(Cc1ccc(N3CCCCC3)cn1)[C@H](CC(C)(C)C)CO2] | +++ | +++ | |
| 3 | 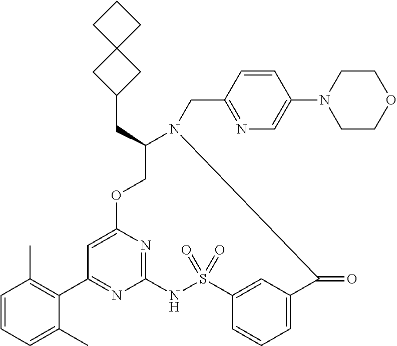 [SMILES: Cc1cccc(C)c1-c1cc2nc(n1)NS(=O)(=O)c1cccc(c1)C(=O)N(Cc1ccc(N3CCOCC3)cn1)[C@H](CC1CC3(CCC3)C1)CO2] | +++ | +++ | |
| 154 | 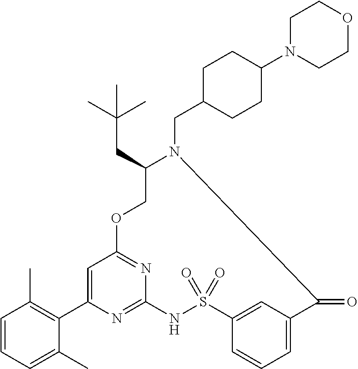 [SMILES: Cc1cccc(C)c1-c1cc2nc(n1)NS(=O)(=O)c1cccc(c1)C(=O)N(CC1CCC(N3CCOCC3)CC1)[C@H](CC(C)(C)C)CO2] | +++ | +++ | |
| 153 | 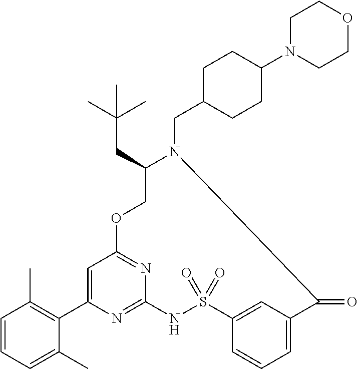 [SMILES: Cc1cccc(C)c1-c1cc2nc(n1)NS(=O)(=O)c1cccc(c1)C(=O)N(CC1CCC(N3CCOCC3)CC1)[C@H](CC(C)(C)C)CO2] | +++ | +++ | |
| 913 | 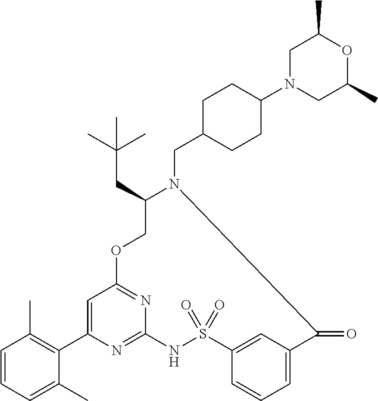 [SMILES: Cc1cccc(C)c1-c1cc2nc(n1)NS(=O)(=O)c1cccc(c1)C(=O)N(CC1CCC(N3C[C@@H](C)O[C@@H](C)C3)CC1)[C@H](CC(C)(C)C)CO2] | +++ | +++ | |
| 914 | 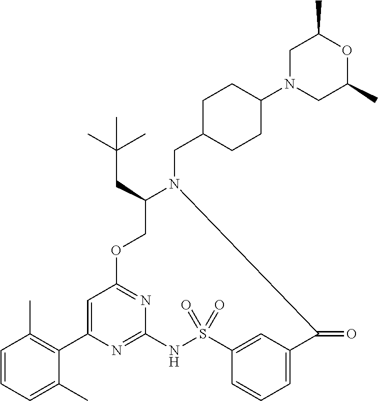 [SMILES: Cc1cccc(C)c1-c1cc2nc(n1)NS(=O)(=O)c1cccc(c1)C(=O)N(CC1CCC(N3C[C@@H](C)O[C@@H](C)C3)CC1)[C@H](CC(C)(C)C)CO2] | +++ | +++ | |
| 501 | 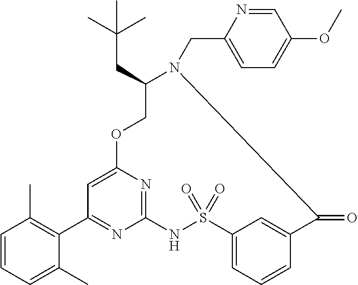 [SMILES: COc1ccc(CN2C(=O)c3cccc(c3)S(=O)(=O)Nc3nc(cc(-c4c(C)cccc4C)n3)OC[C@H]2CC(C)(C)C)nc1] | +++ | +++ | |
| 500 | 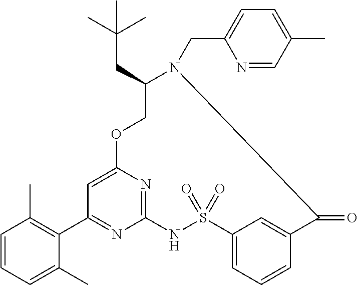 [SMILES: Cc1ccc(CN2C(=O)c3cccc(c3)S(=O)(=O)Nc3nc(cc(-c4c(C)cccc4C)n3)OC[C@H]2CC(C)(C)C)nc1] | +++ | +++ | |
| 56 | 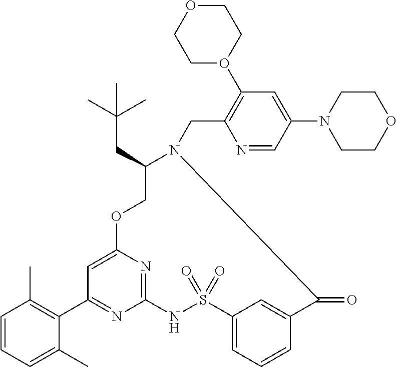 | +++ | +++ | |
| 505 | 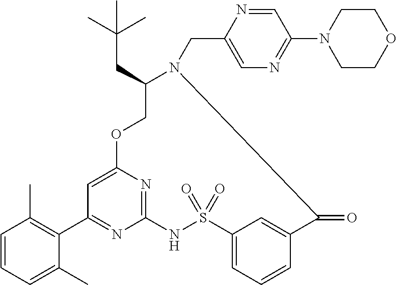 [SMILES: Cc1cccc(C)c1-c1cc2nc(n1)NS(=O)(=O)c1cccc(c1)C(=O)N(Cc1cnc(N3CCOCC3)cn1)[C@H](CC(C)(C)C)CO2] | +++ | +++ | |
| 296 | 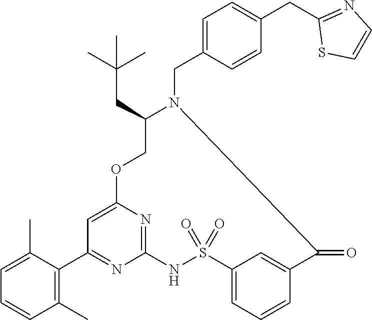 [SMILES: Cc1cccc(C)c1-c1cc2nc(n1)NS(=O)(=O)c1cccc(c1)C(=O)N(Cc1ccc(Cc3nccs3)cc1)[C@H](CC(C)(C)C)CO2] | +++ | +++ | |
| 295 | 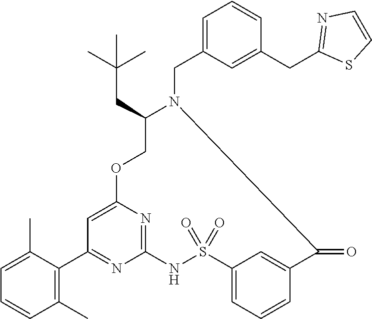 [SMILES: Cc1cccc(C)c1-c1cc2nc(n1)NS(=O)(=O)c1cccc(c1)C(=O)N(Cc1cccc(Cc3nccs3)c1)[C@H](CC(C)(C)C)CO2] | +++ | +++ | |
| 912 | 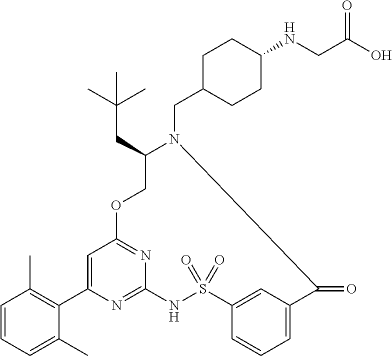 | | | + |
| 911 | 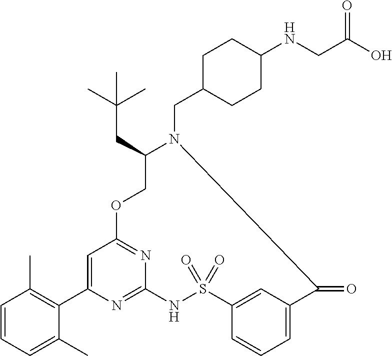 [SMILES: Cc1cccc(C)c1-c1cc2nc(n1)NS(=O)(=O)c1cccc(c1)C(=O)N(CC1CCC(NCC(=O)O)CC1)[C@H](CC(C)(C)C)CO2] | + | ++ | |
| 773 | 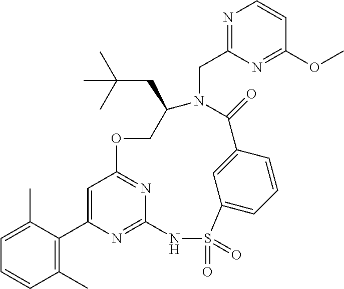 [SMILES: COc1ccnc(CN2C(=O)c3cccc(c3)S(=O)(=O)Nc3nc(cc(-c4c(C)cccc4C)n3)OC[C@H]2CC(C)(C)C)n1] | +++ | +++ | |
| 772 | 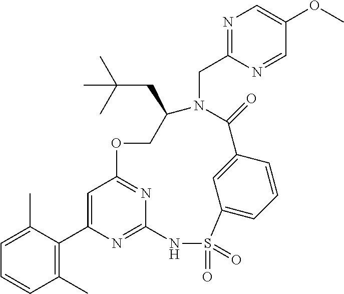 [SMILES: COc1cnc(CN2C(=O)c3cccc(c3)S(=O)(=O)Nc3nc(cc(-c4c(C)cccc4C)n3)OC[C@H]2CC(C)(C)C)nc1] | +++ | +++ | |
| 146 | 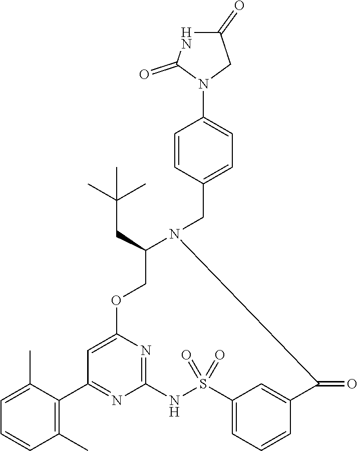 [SMILES: Cc1cccc(C)c1-c1cc2nc(n1)NS(=O)(=O)c1cccc(c1)C(=O)N(Cc1ccc(N3CC(=O)NC3=O)cc1)[C@H](CC(C)(C)C)CO2] | ++ | +++ | |
| 148 | 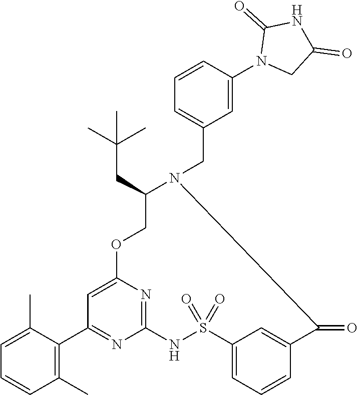 [SMILES: Cc1cccc(C)c1-c1cc2nc(n1)NS(=O)(=O)c1cccc(c1)C(=O)N(Cc1cccc(N3CC(=O)NC3=O)c1)[C@H](CC(C)(C)C)CO2] | ++ | +++ | |
| 910 | 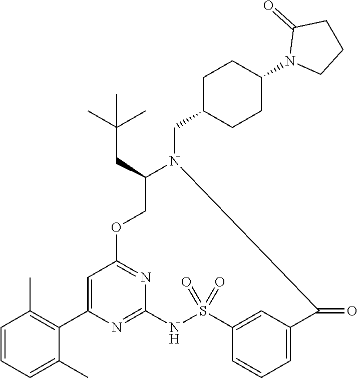 [SMILES: Cc1cccc(C)c1-c1cc2nc(n1)NS(=O)(=O)c1cccc(c1)C(=O)N(C[C@H]1CC[C@@H](N3CCCC3=O)CC1)[C@H](CC(C)(C)C)CO2] | +++ | +++ | |
| 909 | 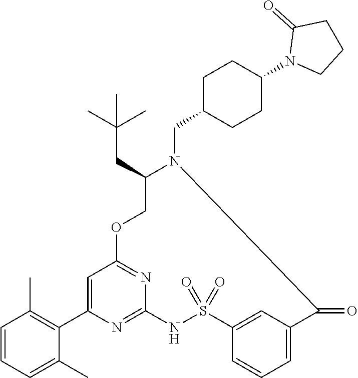 [SMILES: Cc1cccc(C)c1-c1cc2nc(n1)NS(=O)(=O)c1cccc(c1)C(=O)N(C[C@H]1CC[C@@H](N3CCCC3=O)CC1)[C@H](CC(C)(C)C)CO2] | +++ | +++ | |
| 908 | 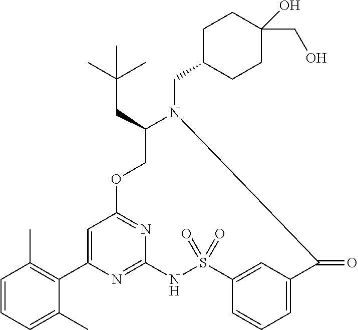 | +++ | +++ | |
| 838 | 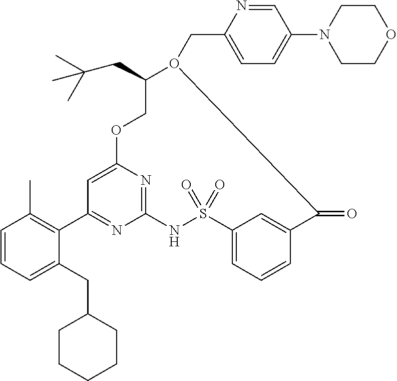 | +++ | +++ | |
| 837 | 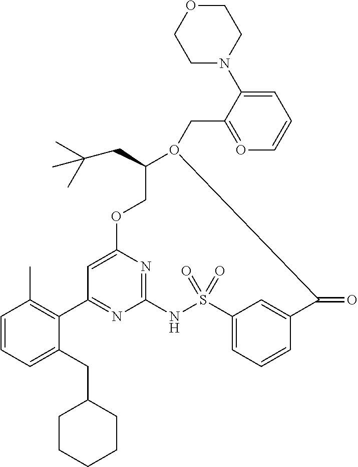 | +++ | +++ | |
| 781 | 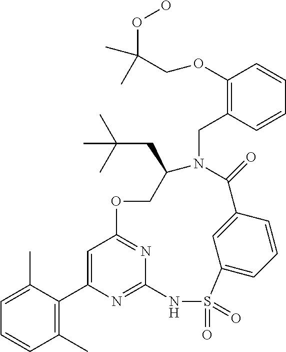 [SMILES: Cc1cccc(C)c1-c1cc2nc(n1)NS(=O)(=O)c1cccc(c1)C(=O)N(Cc1ccccc1OCC(C)(C)OO)[C@H](CC(C)(C)C)CO2] | +++ | +++ | |
| 151 | 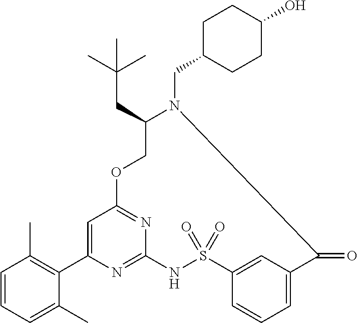 [SMILES: Cc1cccc(C)c1-c1cc2nc(n1)NS(=O)(=O)c1cccc(c1)C(=O)N(C[C@H]1CC[C@@H](O)CC1)[C@H](CC(C)(C)C)CO2] | +++ | +++ | |
| 907 | 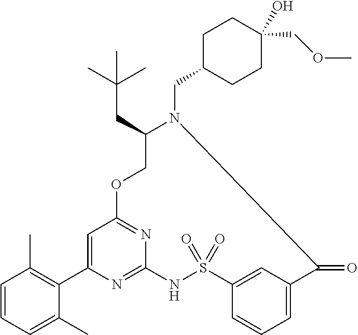 [SMILES: COC[C@]1(O)CC[C@@H](CN2C(=O)c3cccc(c3)S(=O)(=O)Nc3nc(cc(-c4c(C)cccc4C)n3)OC[C@H]2CC(C)(C)C)CC1] | +++ | +++ | |
| 499 | 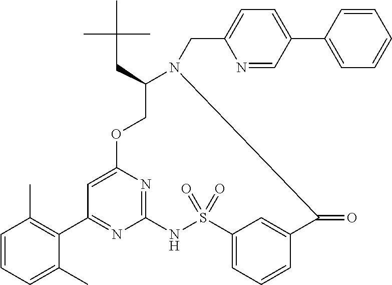 [SMILES: Cc1cccc(C)c1-c1cc2nc(n1)NS(=O)(=O)c1cccc(c1)C(=O)N(Cc1ccc(-c3ccccc3)cn1)[C@H](CC(C)(C)C)CO2] | +++ | +++ | |
| 498 | 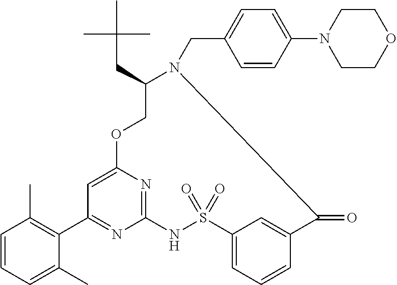 [SMILES: Cc1cccc(C)c1-c1cc2nc(n1)NS(=O)(=O)c1cccc(c1)C(=O)N(Cc1ccc(N3CCOCC3)cc1)[C@H](CC(C)(C)C)CO2] | +++ | +++ | |
| 497 | 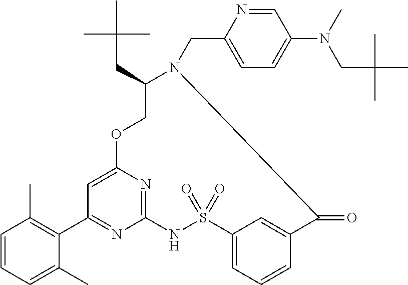 [SMILES: Cc1cccc(C)c1-c1cc2nc(n1)NS(=O)(=O)c1cccc(c1)C(=O)N(Cc1ccc(N(C)CC(C)(C)C)cn1)[C@H](CC(C)(C)C)CO2] | ++ | +++ | |
| 496 | 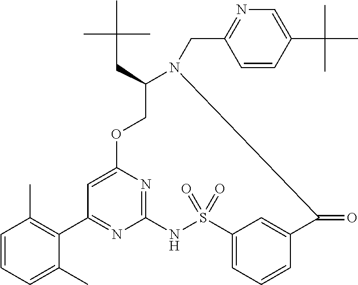 [SMILES: Cc1cccc(C)c1-c1cc2nc(n1)NS(=O)(=O)c1cccc(c1)C(=O)N(Cc1ccc(C(C)(C)C)cn1)[C@H](CC(C)(C)C)CO2] | +++ | +++ | |
| 495 | 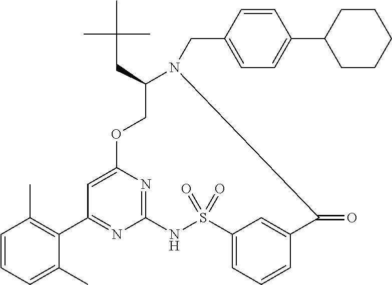 [SMILES: Cc1cccc(C)c1-c1cc2nc(n1)NS(=O)(=O)c1cccc(c1)C(=O)N(Cc1ccc(C3CCCCC3)cc1)[C@H](CC(C)(C)C)CO2] | ++ | +++ | |
| 109 | 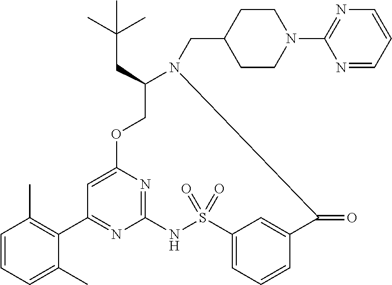 [SMILES: Cc1cccc(C)c1-c1cc2nc(n1)NS(=O)(=O)c1cccc(c1)C(=O)N(CC1CCN(c3ncccn3)CC1)[C@H](CC(C)(C)C)CO2] | +++ | +++ | |
| 795 | 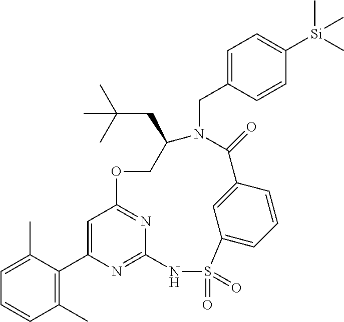 [SMILES: Cc1cccc(C)c1-c1cc2nc(n1)NS(=O)(=O)c1cccc(c1)C(=O)N(Cc1ccc([Si](C)(C)C)cc1)[C@H](CC(C)(C)C)CO2] | | | +++ |
| 771 | 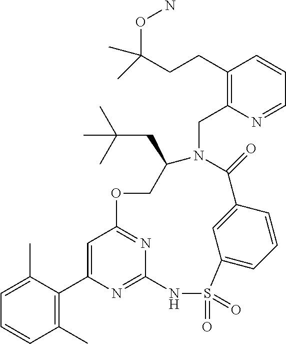 [SMILES: Cc1cccc(C)c1-c1cc2nc(n1)NS(=O)(=O)c1cccc(c1)C(=O)N(Cc1ncccc1CCC(C)(C)ON)[C@H](CC(C)(C)C)CO2] | +++ | +++ | +++ |
| 493 | 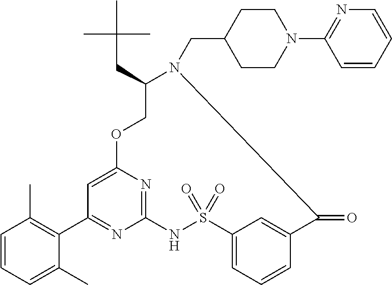 [SMILES: Cc1cccc(C)c1-c1cc2nc(n1)NS(=O)(=O)c1cccc(c1)C(=O)N(CC1CCN(c3ccccn3)CC1)[C@H](CC(C)(C)C)CO2] | +++ | +++ | |
| 57 | 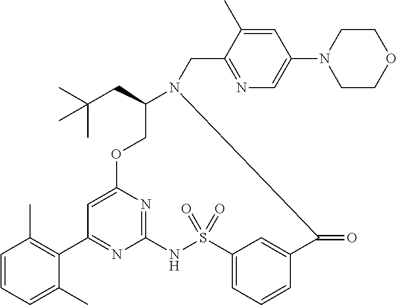 [SMILES: Cc1cc(N2CCOCC2)cnc1CN1C(=O)c2cccc(c2)S(=O)(=O)Nc2nc(cc(-c3c(C)cccc3C)n2)OC[C@H]1CC(C)(C)C] | +++ | +++ | |
| 494 | 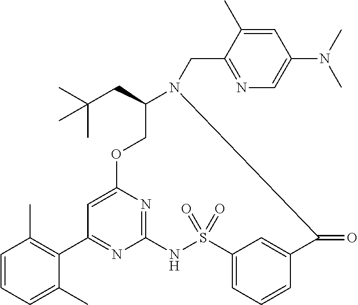 [SMILES: Cc1cc(N(C)C)cnc1CN1C(=O)c2cccc(c2)S(=O)(=O)Nc2nc(cc(-c3c(C)cccc3C)n2)OC[C@H]1CC(C)(C)C] | +++ | +++ | |
| 492 | 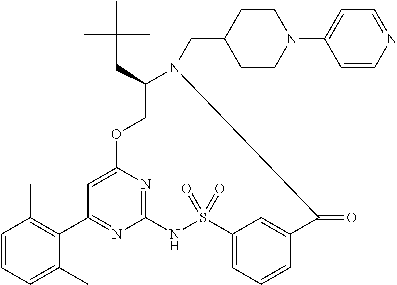 [SMILES: Cc1cccc(C)c1-c1cc2nc(n1)NS(=O)(=O)c1cccc(c1)C(=O)N(CC1CCN(c3ccncc3)CC1)[C@H](CC(C)(C)C)CO2] | | | +++ |
| 770 | 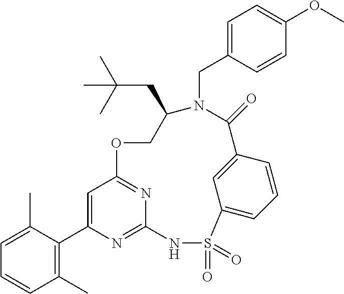 [SMILES: COc1ccc(CN2C(=O)c3cccc(c3)S(=O)(=O)Nc3nc(cc(-c4c(C)cccc4C)n3)OC[C@H]2CC(C)(C)C)cc1] | +++ | +++ | |
| 769 | 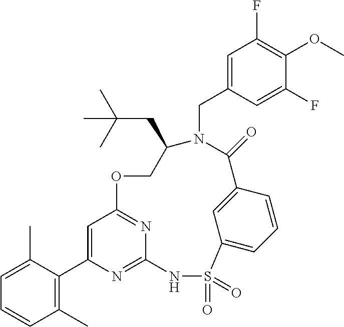 [SMILES: COc1c(F)cc(CN2C(=O)c3cccc(c3)S(=O)(=O)Nc3nc(cc(-c4c(C)cccc4C)n3)OC[C@H]2CC(C)(C)C)cc1F] | | | +++ |
| 768 | 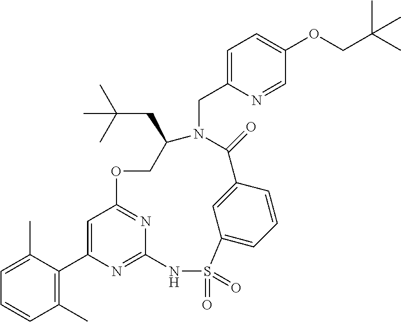 [SMILES: Cc1cccc(C)c1-c1cc2nc(n1)NS(=O)(=O)c1cccc(c1)C(=O)N(Cc1ccc(OCC(C)(C)C)cn1)[C@H](CC(C)(C)C)CO2] | ++ | +++ | |
| 49 | 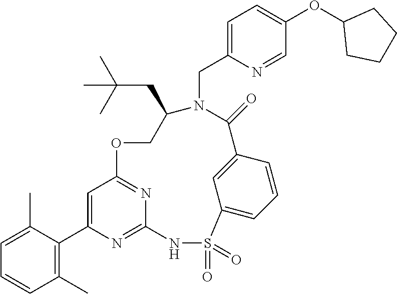 [SMILES: Cc1cccc(C)c1-c1cc2nc(n1)NS(=O)(=O)c1cccc(c1)C(=O)N(Cc1ccc(OC3CCCC3)cn1)[C@H](CC(C)(C)C)CO2] | ++ | +++ | |
| 152 | 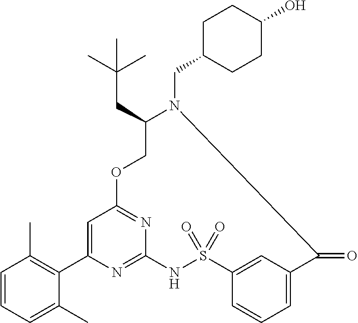 [SMILES: Cc1cccc(C)c1-c1cc2nc(n1)NS(=O)(=O)c1cccc(c1)C(=O)N(C[C@H]1CC[C@@H](O)CC1)[C@H](CC(C)(C)C)CO2] | | | +++ |
| 906 | 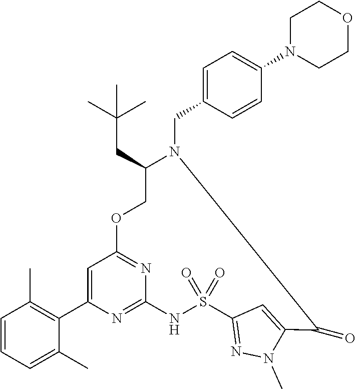 | +++ | +++ | |
| 905 | 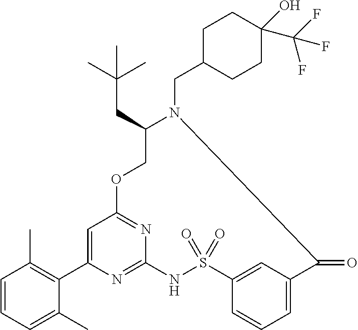 [SMILES: Cc1cccc(C)c1-c1cc2nc(n1)NS(=O)(=O)c1cccc(c1)C(=O)N(CC1CCC(O)(C(F)(F)F)CC1)[C@H](CC(C)(C)C)CO2] | +++ | +++ | |
| 904 | 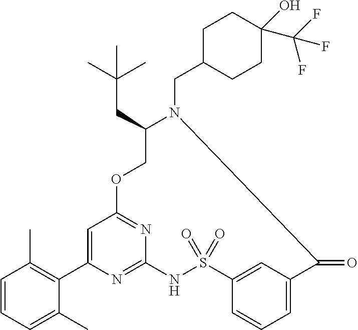 [SMILES: Cc1cccc(C)c1-c1cc2nc(n1)NS(=O)(=O)c1cccc(c1)C(=O)N(CC1CCC(O)(C(F)(F)F)CC1)[C@H](CC(C)(C)C)CO2] | +++ | +++ | |
| 491 | 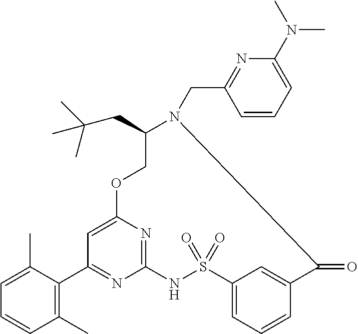 [SMILES: Cc1cccc(C)c1-c1cc2nc(n1)NS(=O)(=O)c1cccc(c1)C(=O)N(Cc1cccc(N(C)C)n1)[C@H](CC(C)(C)C)CO2] | +++ | +++ | +++ |
| 490 | 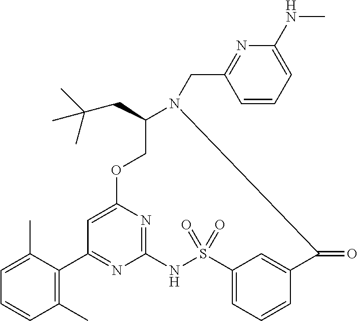 [SMILES: CNc1cccc(CN2C(=O)c3cccc(c3)S(=O)(=O)Nc3nc(cc(-c4c(C)cccc4C)n3)OC[C@H]2CC(C)(C)C)n1] | +++ | +++ | +++ |
| 489 | 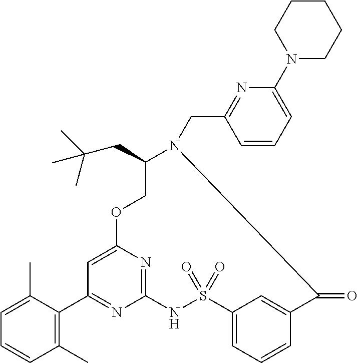 [SMILES: Cc1cccc(C)c1-c1cc2nc(n1)NS(=O)(=O)c1cccc(c1)C(=O)N(Cc1cccc(N3CCCCC3)n1)[C@H](CC(C)(C)C)CO2] | +++ | +++ | |
| 488 | 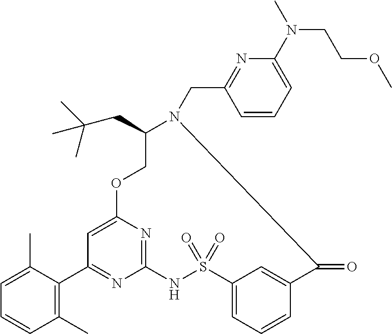 [SMILES: COCCN(C)c1cccc(CN2C(=O)c3cccc(c3)S(=O)(=O)Nc3nc(cc(-c4c(C)cccc4C)n3)OC[C@H]2CC(C)(C)C)n1] | +++ | +++ | |
| 126 | 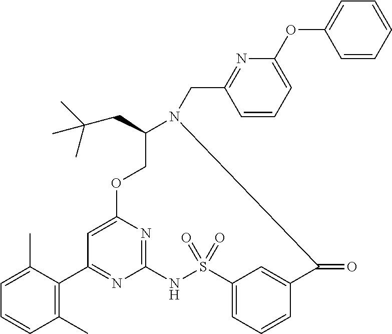 [SMILES: Cc1cccc(C)c1-c1cc2nc(n1)NS(=O)(=O)c1cccc(c1)C(=O)N(Cc1cccc(Oc3ccccc3)n1)[C@H](CC(C)(C)C)CO2] | +++ | +++ | |
| 767 | 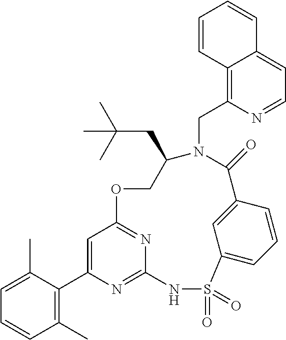 [SMILES: Cc1cccc(C)c1-c1cc2nc(n1)NS(=O)(=O)c1cccc(c1)C(=O)N(Cc1nccc3ccccc13)[C@H](CC(C)(C)C)CO2] | +++ | +++ | |
| 58 | 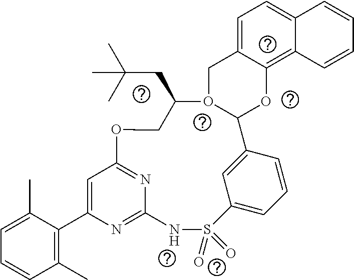 | +++ | +++ | |
| 766 | 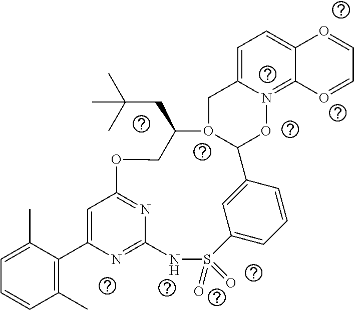 | +++ | +++ | |
| 476 | 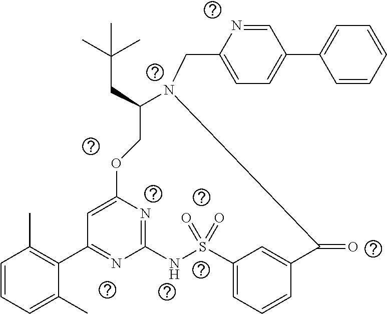 [SMILES: Cc1cccc(C)c1-c1cc2nc(n1)NS(=O)(=O)c1cccc(c1)C(=O)N(Cc1ccc(-c3ccccc3)cn1)[C@H](CC(C)(C)C)CO2] | ++ | +++ | |
| 835 | 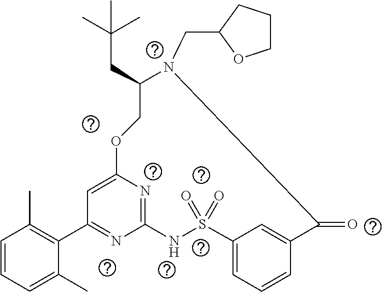 [SMILES: Cc1cccc(C)c1-c1cc2nc(n1)NS(=O)(=O)c1cccc(c1)C(=O)N(CC1CCCO1)[C@H](CC(C)(C)C)CO2] | +++ | +++ | |
| 836 | 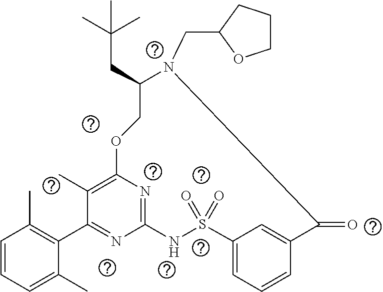 [SMILES: Cc1cccc(C)c1-c1nc2nc(c1C)OC[C@@H](CC(C)(C)C)N(CC1CCCO1)C(=O)c1cccc(c1)S(=O)(=O)N2] | +++ | +++ | |
| 833 | 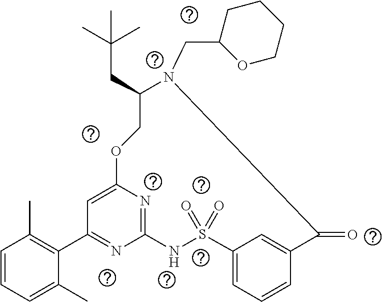 [SMILES: Cc1cccc(C)c1-c1cc2nc(n1)NS(=O)(=O)c1cccc(c1)C(=O)N(CC1CCCCO1)[C@H](CC(C)(C)C)CO2] | +++ | +++ | |
| 834 | 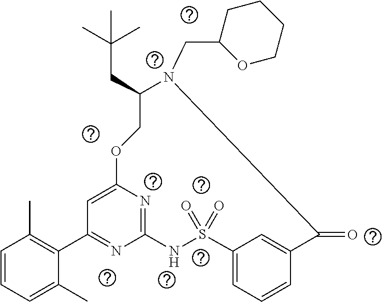 [SMILES: Cc1cccc(C)c1-c1cc2nc(n1)NS(=O)(=O)c1cccc(c1)C(=O)N(CC1CCCCO1)[C@H](CC(C)(C)C)CO2] | +++ | +++ | |
| 764 | 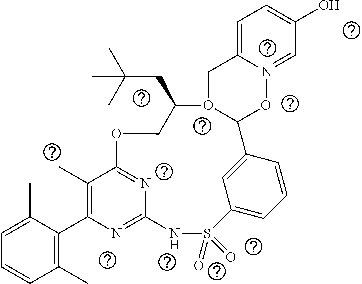 | +++ | +++ | |
| 765 | 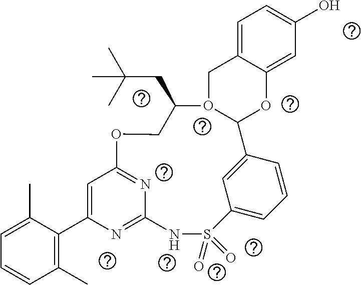 | +++ | +++ | |
| 763 | 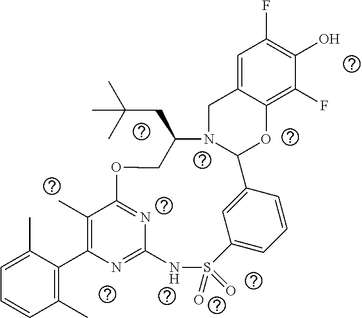 [SMILES: Cc1cccc(C)c1-c1nc2nc(c1C)OC[C@@H](CC(C)(C)C)N1Cc3cc(F)c(O)c(F)c3OC1c1cccc(c1)S(=O)(=O)N2] | | | +++ |
| 294 | 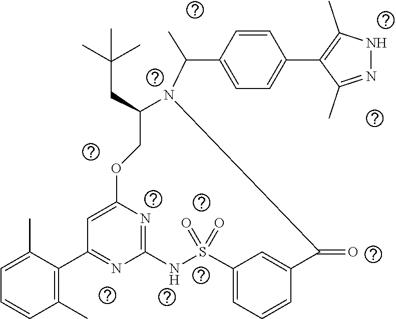 [SMILES: Cc1cccc(C)c1-c1cc2nc(n1)NS(=O)(=O)c1cccc(c1)C(=O)N(C(C)c1ccc(-c3c(C)n[nH]c3C)cc1)[C@H](CC(C)(C)C)CO2] | | | +++ |
| 293 | 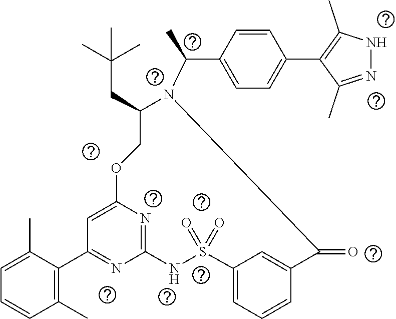 [SMILES: Cc1cccc(C)c1-c1cc2nc(n1)NS(=O)(=O)c1cccc(c1)C(=O)N([C@@H](C)c1ccc(-c3c(C)n[nH]c3C)cc1)[C@H](CC(C)(C)C)CO2] | +++ | +++ | |
| 529 | 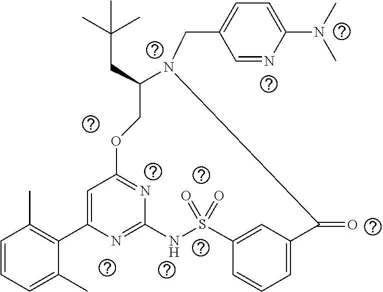 [SMILES: Cc1cccc(C)c1-c1cc2nc(n1)NS(=O)(=O)c1cccc(c1)C(=O)N(Cc1ccc(N(C)C)nc1)[C@H](CC(C)(C)C)CO2] | +++ | +++ | |
| 478 | 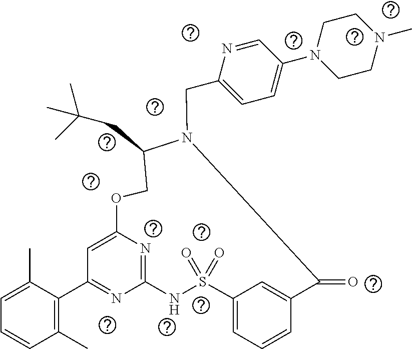 [SMILES: Cc1cccc(C)c1-c1cc2nc(n1)NS(=O)(=O)c1cccc(c1)C(=O)N(Cc1ccc(N3CCN(C)CC3)cn1)[C@H](CC(C)(C)C)CO2] | +++ | +++ | |
| 477 | 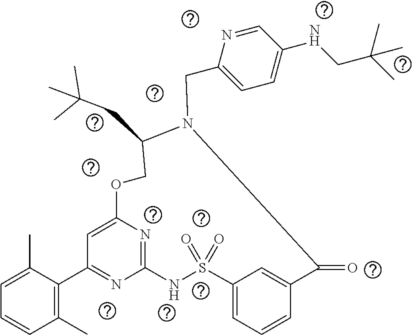 [SMILES: Cc1cccc(C)c1-c1cc2nc(n1)NS(=O)(=O)c1cccc(c1)C(=O)N(Cc1ccc(NCC(C)(C)C)cn1)[C@H](CC(C)(C)C)CO2] | +++ | +++ | |
| 486 | 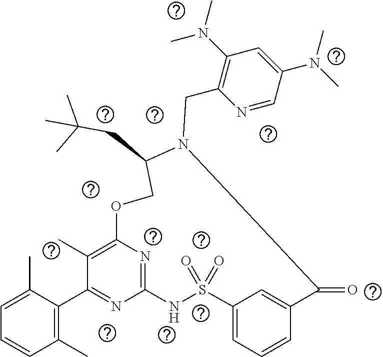 [SMILES: Cc1cccc(C)c1-c1nc2nc(c1C)OC[C@@H](CC(C)(C)C)N(Cc1ncc(N(C)C)cc1N(C)C)C(=O)c1cccc(c1)S(=O)(=O)N2] | | | +++ |
| 111 | 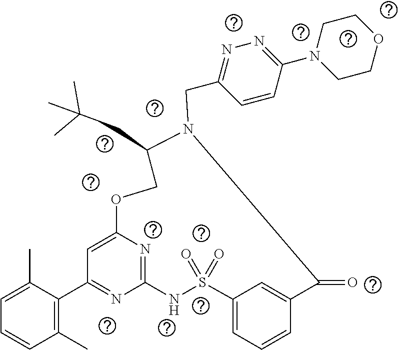 [SMILES: Cc1cccc(C)c1-c1cc2nc(n1)NS(=O)(=O)c1cccc(c1)C(=O)N(Cc1ccc(N3CCOCC3)nn1)[C@H](CC(C)(C)C)CO2] | +++ | +++ | |
| 487 | 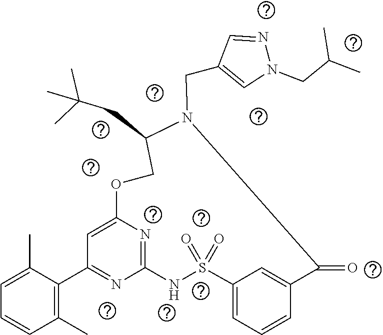 [SMILES: Cc1cccc(C)c1-c1cc2nc(n1)NS(=O)(=O)c1cccc(c1)C(=O)N(Cc1cnn(CC(C)C)c1)[C@H](CC(C)(C)C)CO2] | +++ | +++ | |
| 483 | 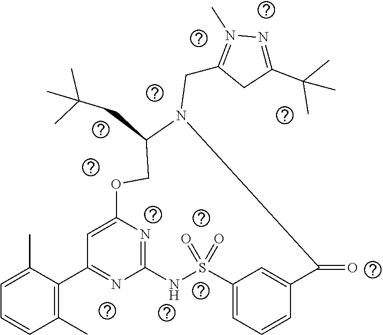 | | | +++ |
| 832 | 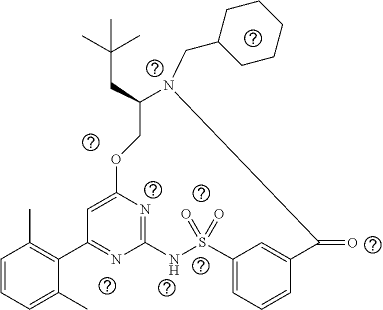 [SMILES: Cc1cccc(C)c1-c1cc2nc(n1)NS(=O)(=O)c1cccc(c1)C(=O)N(CC1CCCCC1)[C@H](CC(C)(C)C)CO2] | | | +++ |
| 1074 | 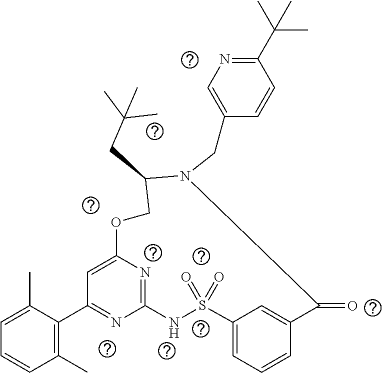 [SMILES: Cc1cccc(C)c1-c1cc2nc(n1)NS(=O)(=O)c1cccc(c1)C(=O)N(Cc1ccc(C(C)(C)C)nc1)[C@H](CC(C)(C)C)CO2] | +++ | +++ | |
| 1073 | 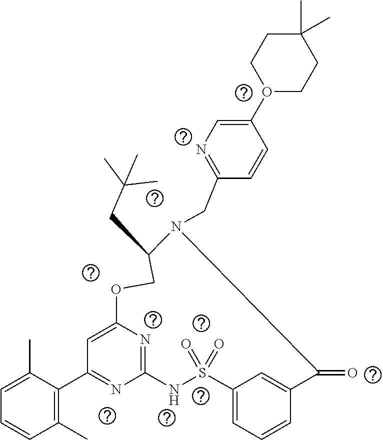 | +++ | +++ | |
| 831 | 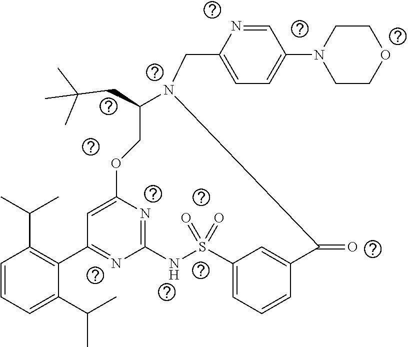 [SMILES: CC(C)c1cccc(C(C)C)c1-c1cc2nc(n1)NS(=O)(=O)c1cccc(c1)C(=O)N(Cc1ccc(N3CCOCC3)cn1)[C@H](CC(C)(C)C)CO2] | +++ | +++ | |
| 830 | 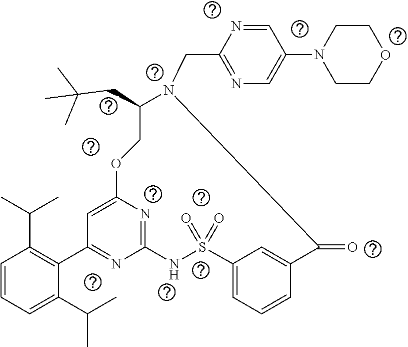 [SMILES: CC(C)c1cccc(C(C)C)c1-c1cc2nc(n1)NS(=O)(=O)c1cccc(c1)C(=O)N(Cc1ncc(N3CCOCC3)cn1)[C@H](CC(C)(C)C)CO2] | +++ | +++ | |
| 759 | 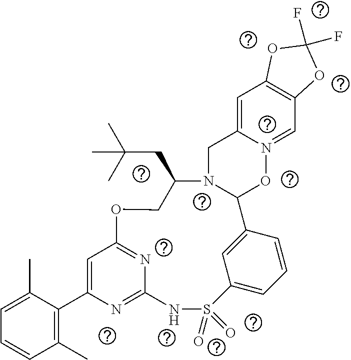 | | | +++ |
| 760 | 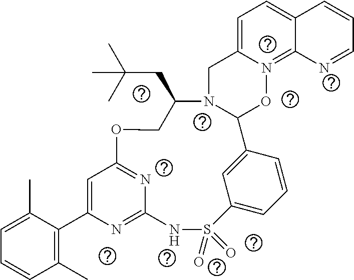 | | | +++ |
| 761 | 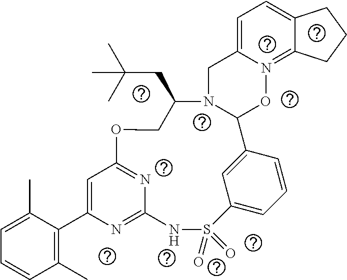 | | | +++ |
| 762 | 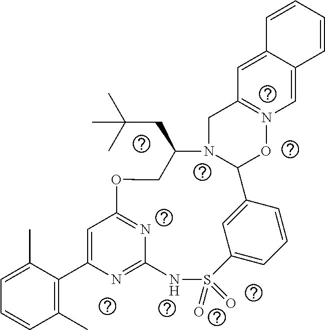 | | | +++ |
| 829 | 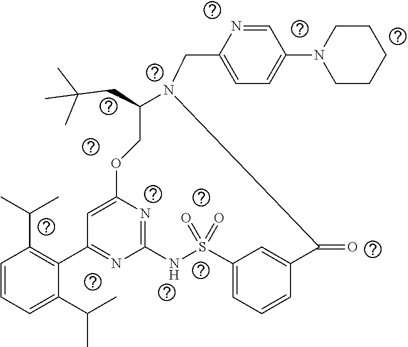 [SMILES: CC(C)c1cccc(C(C)C)c1-c1cc2nc(n1)NS(=O)(=O)c1cccc(c1)C(=O)N(Cc1ccc(N3CCCCC3)cn1)[C@H](CC(C)(C)C)CO2] | | | +++ |
| 50 | 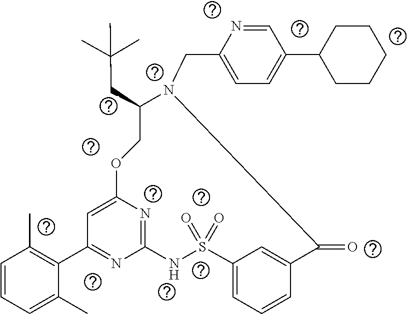 [SMILES: Cc1cccc(C)c1-c1cc2nc(n1)NS(=O)(=O)c1cccc(c1)C(=O)N(Cc1ccc(C3CCCCC3)cn1)[C@H](CC(C)(C)C)CO2] | ++ | +++ | |
| 292 | 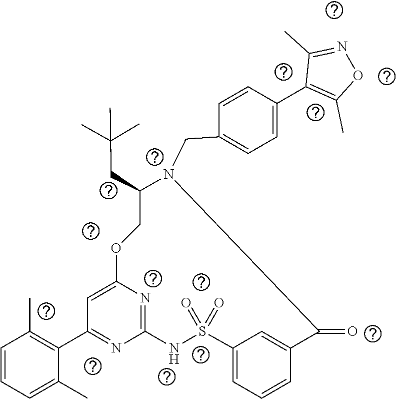 [SMILES: Cc1cccc(C)c1-c1cc2nc(n1)NS(=O)(=O)c1cccc(c1)C(=O)N(Cc1ccc(-c3c(C)noc3C)cc1)[C@H](CC(C)(C)C)CO2] | +++ | +++ | |
| 528 | 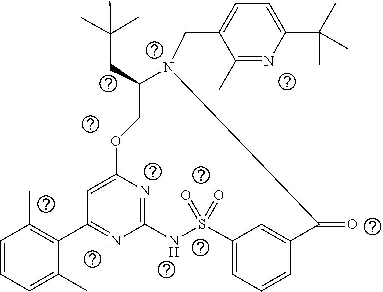 [SMILES: Cc1cccc(C)c1-c1cc2nc(n1)NS(=O)(=O)c1cccc(c1)C(=O)N(Cc1ccc(C(C)(C)C)nc1C)[C@H](CC(C)(C)C)CO2] | +++ | +++ | |
| 291 | 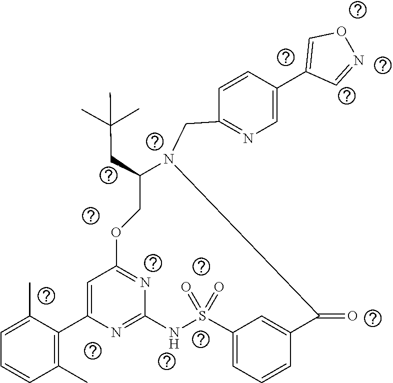 [SMILES: Cc1cccc(C)c1-c1cc2nc(n1)NS(=O)(=O)c1cccc(c1)C(=O)N(Cc1ccc(-c3cnoc3)cn1)[C@H](CC(C)(C)C)CO2] | +++ | +++ | |
| 756 | 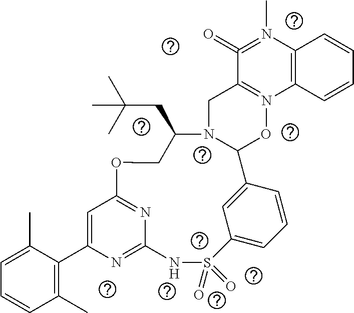 | +++ | +++ | |
| 485 | 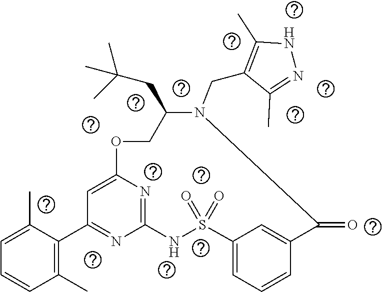 [SMILES: Cc1cccc(C)c1-c1cc2nc(n1)NS(=O)(=O)c1cccc(c1)C(=O)N(Cc1c(C)n[nH]c1C)[C@H](CC(C)(C)C)CO2] | | | +++ |
| 482 | 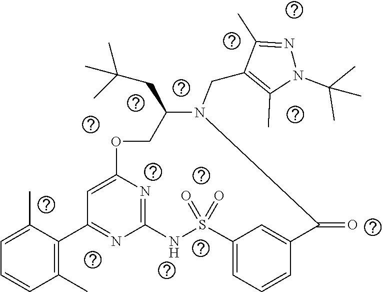 [SMILES: Cc1cccc(C)c1-c1cc2nc(n1)NS(=O)(=O)c1cccc(c1)C(=O)N(Cc1c(C)nn(C(C)(C)C)c1C)[C@H](CC(C)(C)C)CO2] | | | +++ |
| 134 | 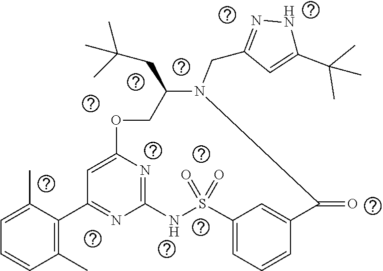 [SMILES: Cc1cccc(C)c1-c1cc2nc(n1)NS(=O)(=O)c1cccc(c1)C(=O)N(Cc1cc(C(C)(C)C)[nH]n1)[C@H](CC(C)(C)C)CO2] | +++ | +++ | |
| 481 | 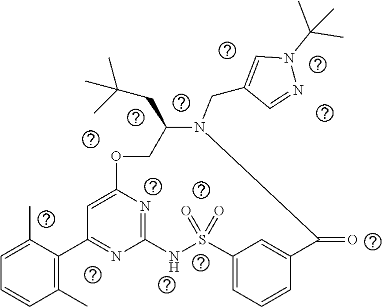 [SMILES: Cc1cccc(C)c1-c1cc2nc(n1)NS(=O)(=O)c1cccc(c1)C(=O)N(Cc1cnn(C(C)(C)C)c1)[C@H](CC(C)(C)C)CO2] | +++ | +++ | |
| 480 | 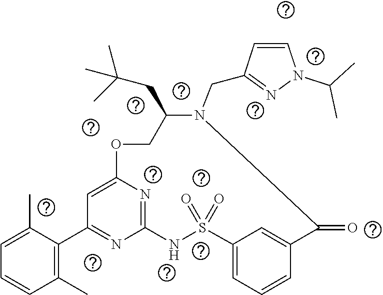 [SMILES: Cc1cccc(C)c1-c1cc2nc(n1)NS(=O)(=O)c1cccc(c1)C(=O)N(Cc1ccn(C(C)C)n1)[C@H](CC(C)(C)C)CO2] | +++ | +++ | |
| 479 | 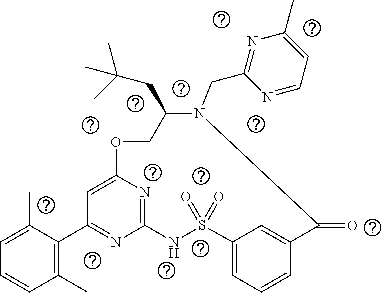 [SMILES: Cc1ccnc(CN2C(=O)c3cccc(c3)S(=O)(=O)Nc3nc(cc(-c4c(C)cccc4C)n3)OC[C@H]2CC(C)(C)C)n1] | +++ | +++ | +++ |
| 484 | 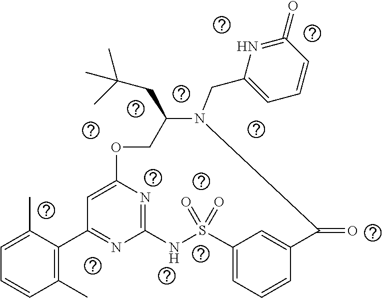 | | | ++ |
| 828 | 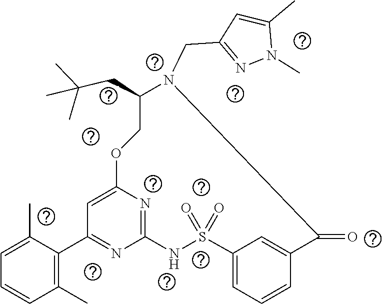 [SMILES: Cc1cccc(C)c1-c1cc2nc(n1)NS(=O)(=O)c1cccc(c1)C(=O)N(Cc1cc(C)n(C)n1)[C@H](CC(C)(C)C)CO2] | | | +++ |
| 827 | 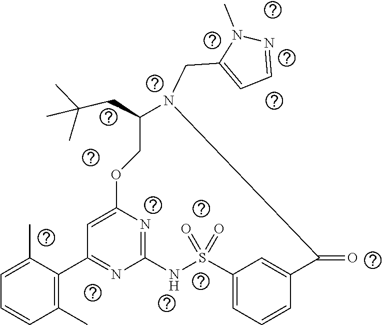 [SMILES: Cc1cccc(C)c1-c1cc2nc(n1)NS(=O)(=O)c1cccc(c1)C(=O)N(Cc1ccnn1C)[C@H](CC(C)(C)C)CO2] | | | +++ |
| 826 | 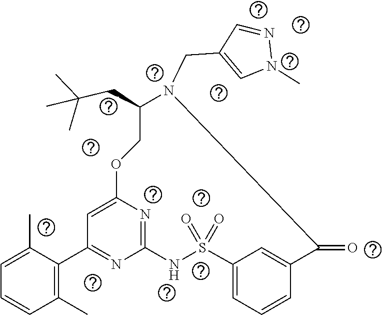 [SMILES: Cc1cccc(C)c1-c1cc2nc(n1)NS(=O)(=O)c1cccc(c1)C(=O)N(Cc1cnn(C)c1)[C@H](CC(C)(C)C)CO2] | | | +++ |
| 825 | 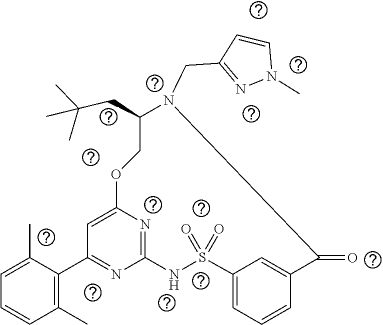 [SMILES: Cc1cccc(C)c1-c1cc2nc(n1)NS(=O)(=O)c1cccc(c1)C(=O)N(Cc1ccn(C)n1)[C@H](CC(C)(C)C)CO2] | | | +++ |
| 824 | 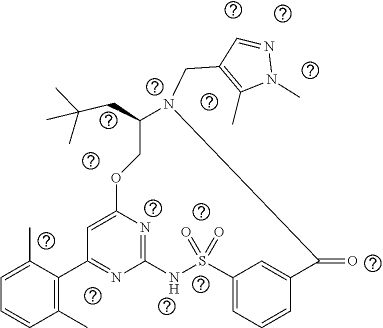 [SMILES: Cc1cccc(C)c1-c1cc2nc(n1)NS(=O)(=O)c1cccc(c1)C(=O)N(Cc1cnn(C)c1C)[C@H](CC(C)(C)C)CO2] | | | +++ |
| 823 | 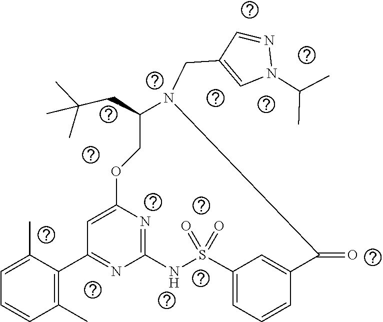 [SMILES: Cc1cccc(C)c1-c1cc2nc(n1)NS(=O)(=O)c1cccc(c1)C(=O)N(Cc1cnn(C(C)C)c1)[C@H](CC(C)(C)C)CO2] | +++ | +++ | +++ |
| 822 | 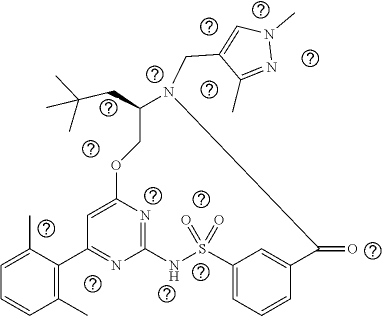 [SMILES: Cc1cccc(C)c1-c1cc2nc(n1)NS(=O)(=O)c1cccc(c1)C(=O)N(Cc1cn(C)nc1C)[C@H](CC(C)(C)C)CO2] | | | +++ |
| 757 | 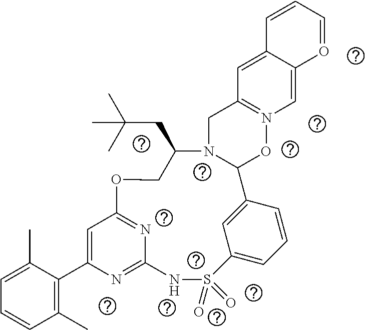 | +++ | +++ | |
| 1072 | 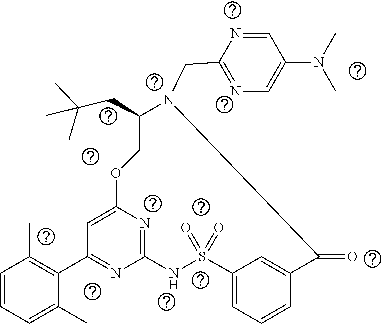 [SMILES: Cc1cccc(C)c1-c1cc2nc(n1)NS(=O)(=O)c1cccc(c1)C(=O)N(Cc1ncc(N(C)C)cn1)[C@H](CC(C)(C)C)CO2] | +++ | +++ | |
| 475 | 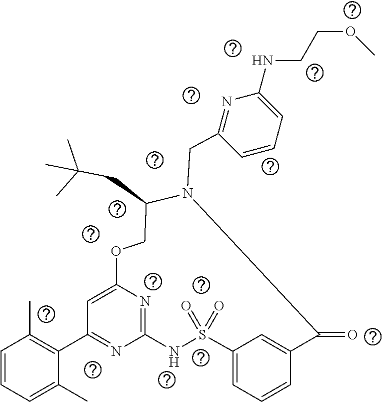 [SMILES: COCCNc1cccc(CN2C(=O)c3cccc(c3)S(=O)(=O)Nc3nc(cc(-c4c(C)cccc4C)n3)OC[C@H]2CC(C)(C)C)n1] | +++ | +++ | |
| 472 | 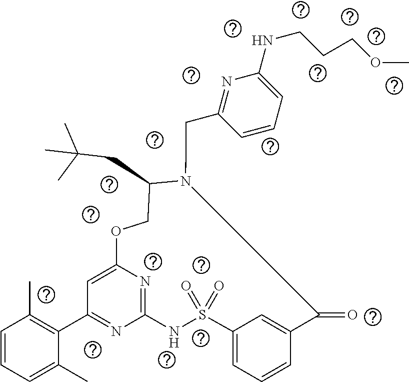 [SMILES: COCCCNc1cccc(CN2C(=O)c3cccc(c3)S(=O)(=O)Nc3nc(cc(-c4c(C)cccc4C)n3)OC[C@H]2CC(C)(C)C)n1] | +++ | +++ | +++ |
| 127 | 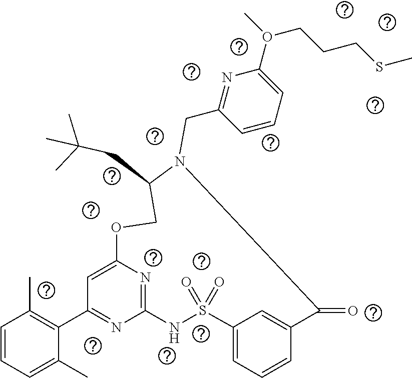 | +++ | +++ | |
| 471 | 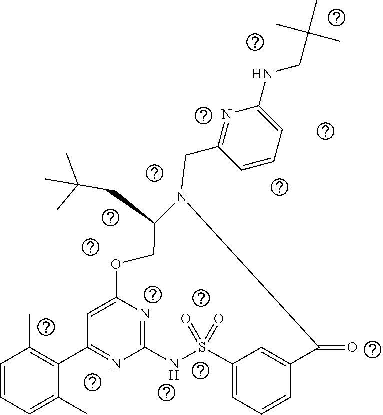 [SMILES: Cc1cccc(C)c1-c1cc2nc(n1)NS(=O)(=O)c1cccc(c1)C(=O)N(Cc1cccc(NCC(C)(C)C)n1)[C@H](CC(C)(C)C)CO2] | +++ | +++ | |
| 469 | 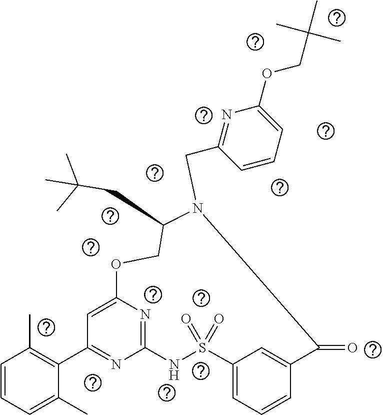 [SMILES: Cc1cccc(C)c1-c1cc2nc(n1)NS(=O)(=O)c1cccc(c1)C(=O)N(Cc1cccc(OCC(C)(C)C)n1)[C@H](CC(C)(C)C)CO2] | +++ | +++ | |
| 470 | 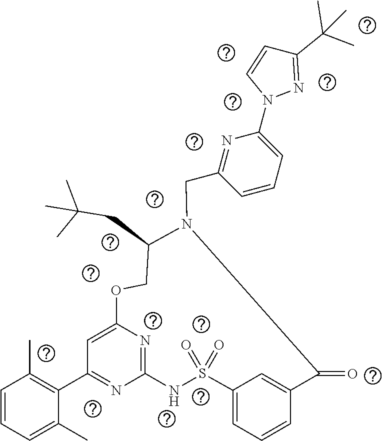 [SMILES: Cc1cccc(C)c1-c1cc2nc(n1)NS(=O)(=O)c1cccc(c1)C(=O)N(Cc1cccc(-n3ccc(C(C)(C)C)n3)n1)[C@H](CC(C)(C)C)CO2] | +++ | +++ | |
| 467 | 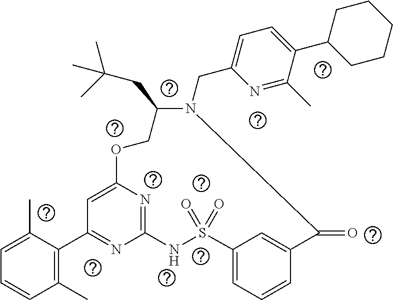 [SMILES: Cc1cccc(C)c1-c1cc2nc(n1)NS(=O)(=O)c1cccc(c1)C(=O)N(Cc1ccc(C3CCCCC3)c(C)n1)[C@H](CC(C)(C)C)CO2] | | | +++ |
| 468 | 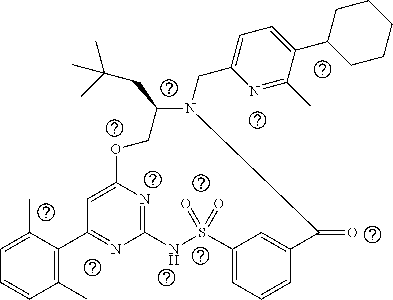 [SMILES: Cc1cccc(C)c1-c1cc2nc(n1)NS(=O)(=O)c1cccc(c1)C(=O)N(Cc1ccc(C3CCCCC3)c(C)n1)[C@H](CC(C)(C)C)CO2] | | | +++ |
| 128 | 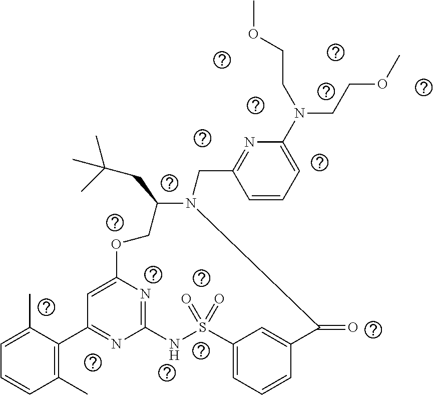 [SMILES: COCCN(CCOC)c1cccc(CN2C(=O)c3cccc(c3)S(=O)(=O)Nc3nc(cc(-c4c(C)cccc4C)n3)OC[C@H]2CC(C)(C)C)n1] | +++ | +++ | +++ |
| 129 | 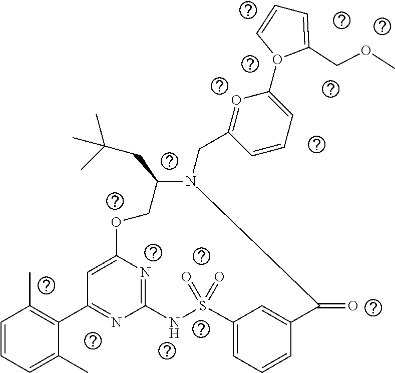 | +++ | +++ | +++ |
| 466 | 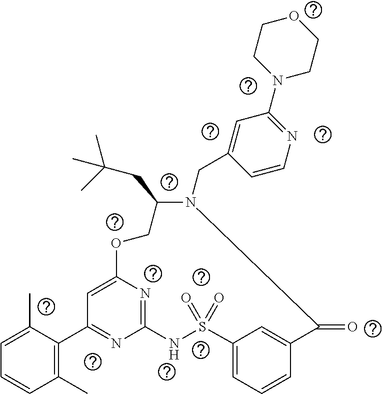 [SMILES: Cc1cccc(C)c1-c1cc2nc(n1)NS(=O)(=O)c1cccc(c1)C(=O)N(Cc1ccnc(N3CCOCC3)c1)[C@H](CC(C)(C)C)CO2] | +++ | +++ | +++ |
| 755 | 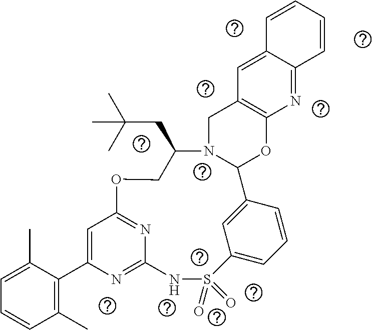 [SMILES: Cc1cccc(C)c1-c1cc2nc(n1)NS(=O)(=O)c1cccc(c1)C1Oc3nc4ccccc4cc3CN1[C@H](CC(C)(C)C)CO2] | | | +++ |
| 754 | 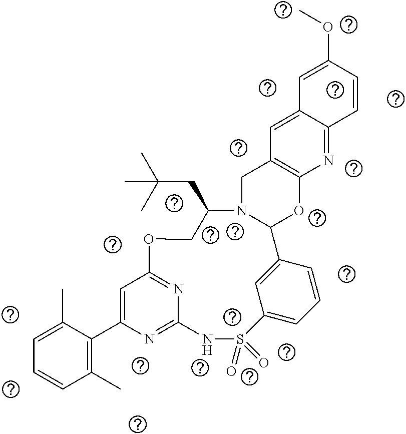 [SMILES: COc1ccc2nc3c(cc2c1)CN1C(O3)c2cccc(c2)S(=O)(=O)Nc2nc(cc(-c3c(C)cccc3C)n2)OC[C@H]1CC(C)(C)C] | | | +++ |
| 753 | 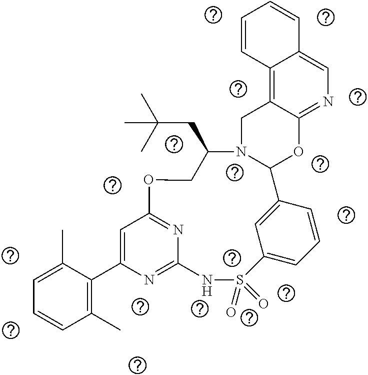 [SMILES: Cc1cccc(C)c1-c1cc2nc(n1)NS(=O)(=O)c1cccc(c1)C1Oc3ncc4ccccc4c3CN1[C@H](CC(C)(C)C)CO2] | | | ++ |
| 752 | 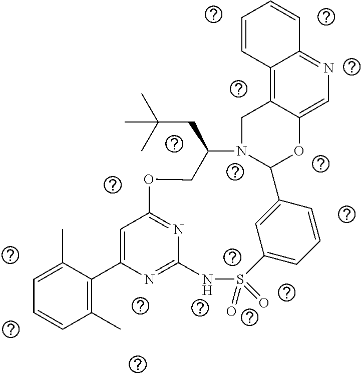 [SMILES: Cc1cccc(C)c1-c1cc2nc(n1)NS(=O)(=O)c1cccc(c1)C1Oc3cnc4ccccc4c3CN1[C@H](CC(C)(C)C)CO2] | | | + |
| 751 | 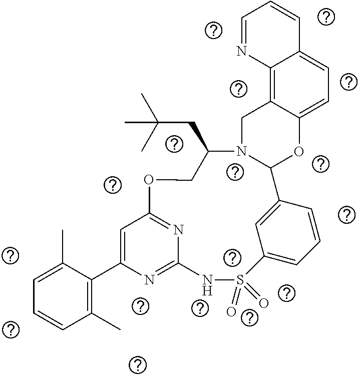 [SMILES: Cc1cccc(C)c1-c1cc2nc(n1)NS(=O)(=O)c1cccc(c1)C1Oc3ccc4cccnc4c3CN1[C@H](CC(C)(C)C)CO2] | | | +++ |
| 750 | 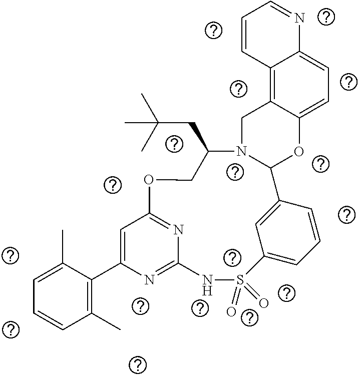 [SMILES: Cc1cccc(C)c1-c1cc2nc(n1)NS(=O)(=O)c1cccc(c1)C1Oc3ccc4ncccc4c3CN1[C@H](CC(C)(C)C)CO2] | | | +++ |
| 290 | 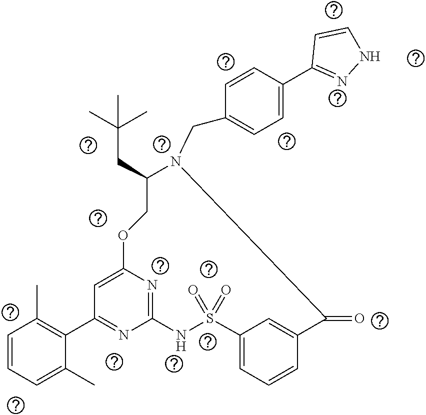 [SMILES: Cc1cccc(C)c1-c1cc2nc(n1)NS(=O)(=O)c1cccc(c1)C(=O)N(Cc1ccc(-c3cc[nH]n3)cc1)[C@H](CC(C)(C)C)CO2] | | | +++ |
| 289 | 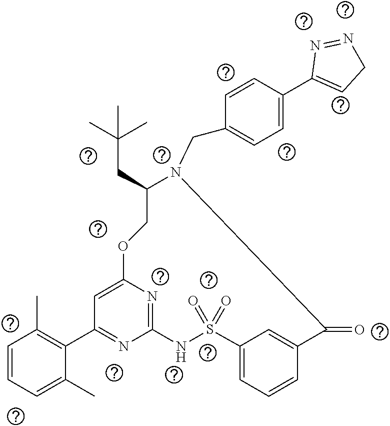 [SMILES: Cc1cccc(C)c1-c1cc2nc(n1)NS(=O)(=O)c1cccc(c1)C(=O)N(Cc1ccc(C3=CCN=N3)cc1)[C@H](CC(C)(C)C)CO2] | +++ | +++ | |
| 288 | 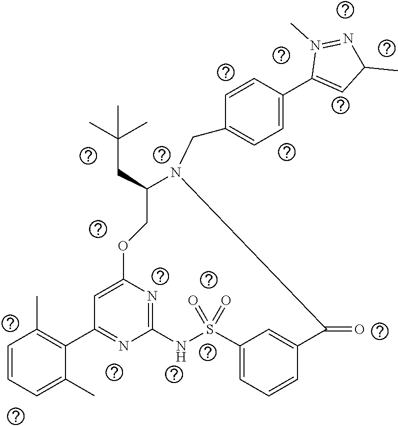 | +++ | +++ | |
| 287 | 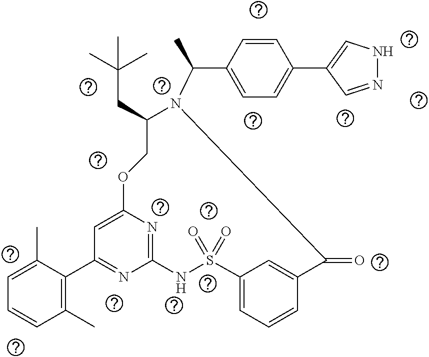 [SMILES: Cc1cccc(C)c1-c1cc2nc(n1)NS(=O)(=O)c1cccc(c1)C(=O)N([C@@H](C)c1ccc(-c3cn[nH]c3)cc1)[C@H](CC(C)(C)C)CO2] | +++ | +++ | |
| 139 | 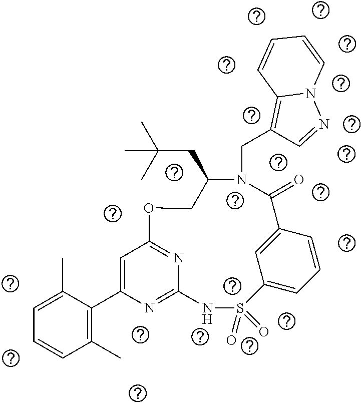 [SMILES: Cc1cccc(C)c1-c1cc2nc(n1)NS(=O)(=O)c1cccc(c1)C(=O)N(Cc1cnn3ccccc13)[C@H](CC(C)(C)C)CO2] | | | +++ |
| 114 | 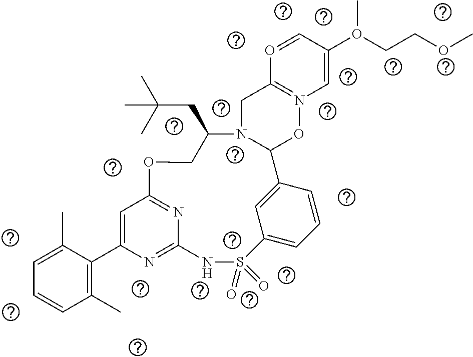 | +++ | +++ | |
| 749 | 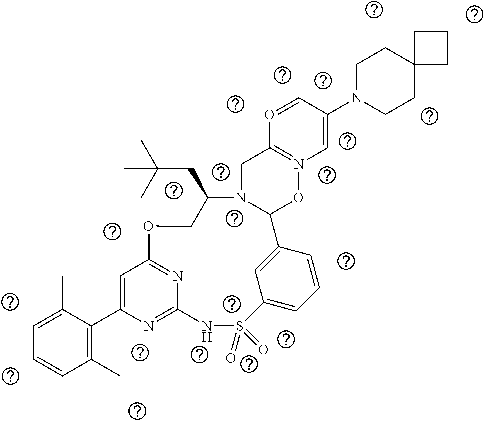 | +++ | +++ | |
| 115 | 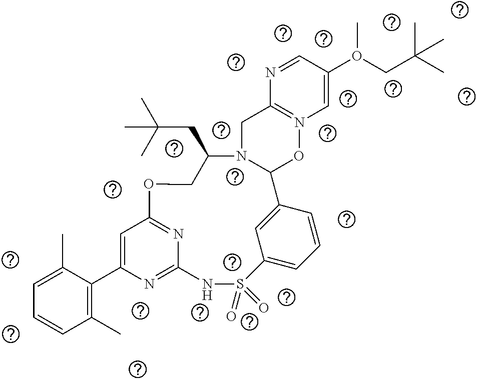 | +++ | +++ | |

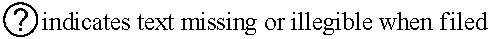

8. Biological Activity Data

Table 11 represents CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” % Activity: +++ is >60%; ++ is 30-60%; + is <30%).

TABLE 11

| | | | HBE | HBE |
|---|---|---|---|---|
| | | HBE | Max | Activity at |
| Cmpd | | EC₅₀ | Activity | 10 μM |
| Number | Structure | (μM) | (%) | (%) |
| 116 | 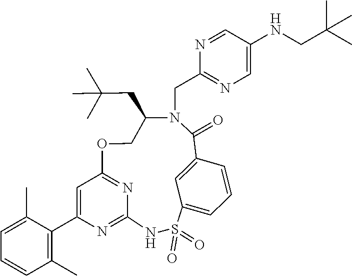 [SMILES: Cc1cccc(C)c1-c1cc2nc(n1)NS(=O)(=O)c1cccc(c1)C(=O)N(Cc1ncc(NCC(C)(C)C)cn1)[C@H](CC(C)(C)C)CO2] | +++ | +++ | +++ |
| 748 | 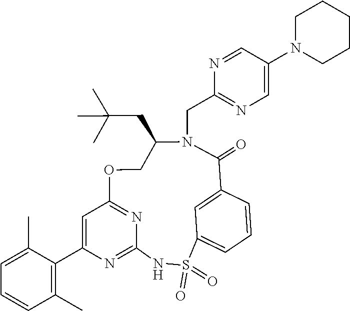 [SMILES: Cc1cccc(C)c1-c1cc2nc(n1)NS(=O)(=O)c1cccc(c1)C(=O)N(Cc1ncc(N3CCCCC3)cn1)[C@H](CC(C)(C)C)CO2] | +++ | +++ | +++ |
| 747 | 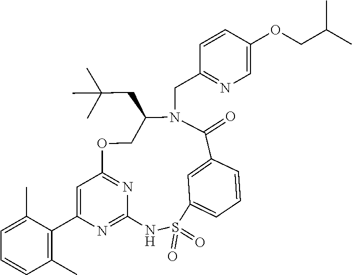 [SMILES: Cc1cccc(C)c1-c1cc2nc(n1)NS(=O)(=O)c1cccc(c1)C(=O)N(Cc1ccc(OCC(C)C)cn1)[C@H](CC(C)(C)C)CO2] | | | +++ |
| 746 | 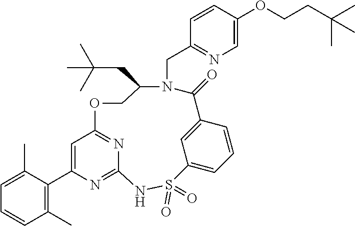 [SMILES: Cc1cccc(C)c1-c1cc2nc(n1)NS(=O)(=O)c1cccc(c1)C(=O)N(Cc1ccc(OCCC(C)(C)C)cn1)[C@H](CC(C)(C)C)CO2] | | | +++ |
| 821 | 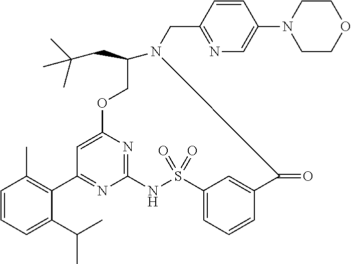 [SMILES: Cc1cccc(C(C)C)c1-c1cc2nc(n1)NS(=O)(=O)c1cccc(c1)C(=O)N(Cc1ccc(N3CCOCC3)cn1)[C@H](CC(C)(C)C)CO2] | +++ | +++ | +++ |
| 474 | 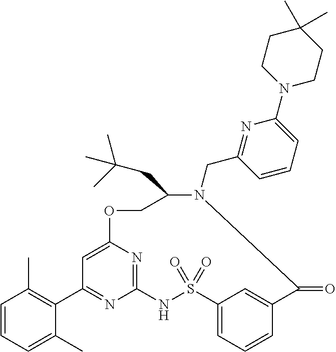 [SMILES: Cc1cccc(C)c1-c1cc2nc(n1)NS(=O)(=O)c1cccc(c1)C(=O)N(Cc1cccc(N3CCC(C)(C)CC3)n1)[C@H](CC(C)(C)C)CO2] | +++ | +++ | |
| 130 | 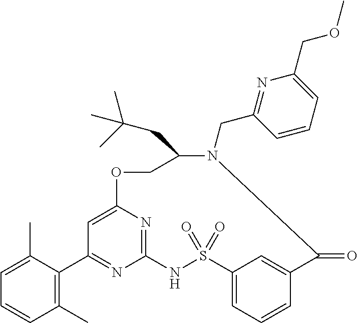 [SMILES: COCc1cccc(CN2C(=O)c3cccc(c3)S(=O)(=O)Nc3nc(cc(-c4c(C)cccc4C)n3)OC[C@H]2CC(C)(C)C)n1] | +++ | +++ | |
| 110 | 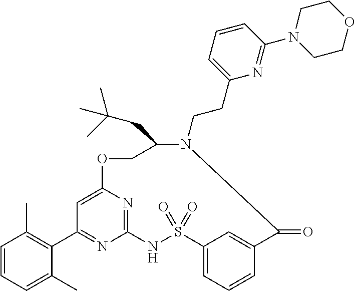 [SMILES: Cc1cccc(C)c1-c1cc2nc(n1)NS(=O)(=O)c1cccc(c1)C(=O)N(CCc1cccc(N3CCOCC3)n1)[C@H](CC(C)(C)C)CO2] | +++ | +++ | +++ |
| 464 | 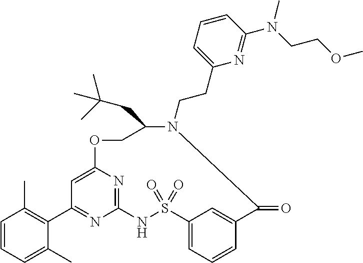 [SMILES: COCCN(C)c1cccc(CCN2C(=O)c3cccc(c3)S(=O)(=O)Nc3nc(cc(-c4c(C)cccc4C)n3)OC[C@H]2CC(C)(C)C)n1] | +++ | +++ | |
| 7 | 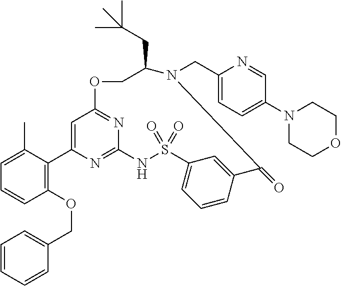 [SMILES: Cc1cccc(OCc2ccccc2)c1-c1cc2nc(n1)NS(=O)(=O)c1cccc(c1)C(=O)N(Cc1ccc(N3CCOCC3)cn1)[C@H](CC(C)(C)C)CO2] | +++ | +++ | +++ |
| 774 | 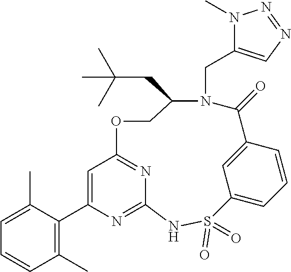 [SMILES: Cc1cccc(C)c1-c1cc2nc(n1)NS(=O)(=O)c1cccc(c1)C(=O)N(Cc1cnnn1C)[C@H](CC(C)(C)C)CO2] | | | +++ |
| 140 | 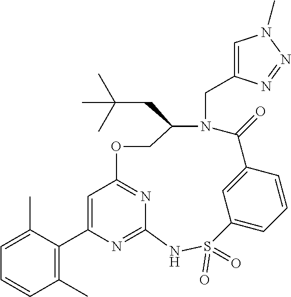 [SMILES: Cc1cccc(C)c1-c1cc2nc(n1)NS(=O)(=O)c1cccc(c1)C(=O)N(Cc1cn(C)nn1)[C@H](CC(C)(C)C)CO2] | | | +++ |
| 51 | 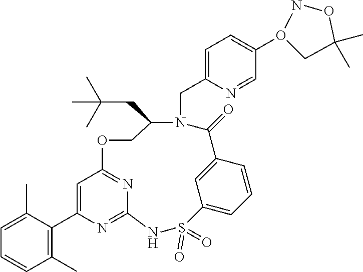 | +++ | +++ | +++ |
| 6 | 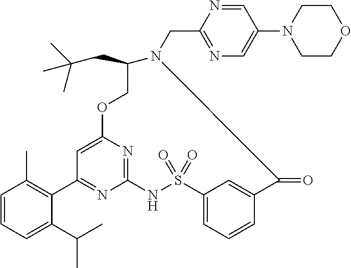 [SMILES: Cc1cccc(C(C)C)c1-c1cc2nc(n1)NS(=O)(=O)c1cccc(c1)C(=O)N(Cc1ncc(N3CCOCC3)cn1)[C@H](CC(C)(C)C)CO2] | +++ | +++ | |
| 465 | 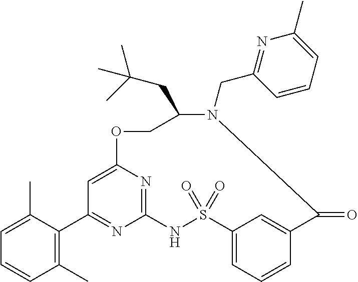 [SMILES: Cc1cccc(CN2C(=O)c3cccc(c3)S(=O)(=O)Nc3nc(cc(-c4c(C)cccc4C)n3)OC[C@H]2CC(C)(C)C)n1] | +++ | +++ | |
| 112 | 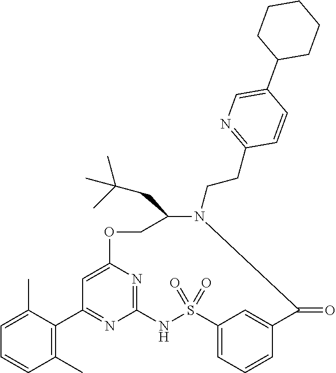 [SMILES: Cc1cccc(C)c1-c1cc2nc(n1)NS(=O)(=O)c1cccc(c1)C(=O)N(CCc1ccc(C3CCCCC3)cn1)[C@H](CC(C)(C)C)CO2] | +++ | +++ | |
| 473 | 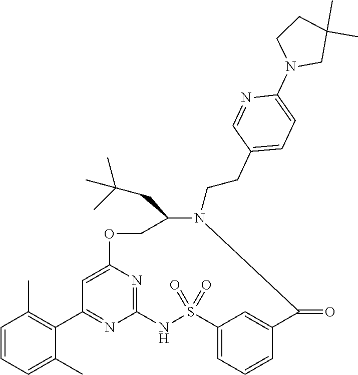 [SMILES: Cc1cccc(C)c1-c1cc2nc(n1)NS(=O)(=O)c1cccc(c1)C(=O)N(CCc1ccc(N3CCC(C)(C)C3)nc1)[C@H](CC(C)(C)C)CO2] | +++ | +++ | |
| 745 | 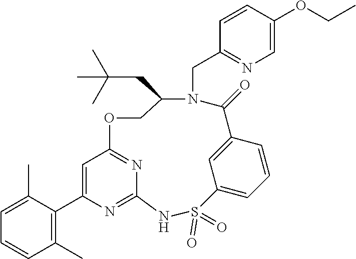 [SMILES: CCOc1ccc(CN2C(=O)c3cccc(c3)S(=O)(=O)Nc3nc(cc(-c4c(C)cccc4C)n3)OC[C@H]2CC(C)(C)C)nc1] | +++ | +++ | |
| 744 | 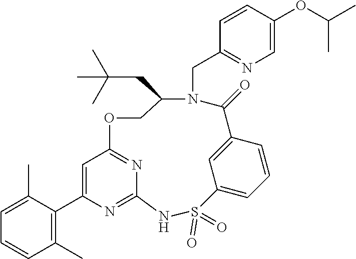 [SMILES: Cc1cccc(C)c1-c1cc2nc(n1)NS(=O)(=O)c1cccc(c1)C(=O)N(Cc1ccc(OC(C)C)cn1)[C@H](CC(C)(C)C)CO2] | +++ | +++ | |
| 743 | 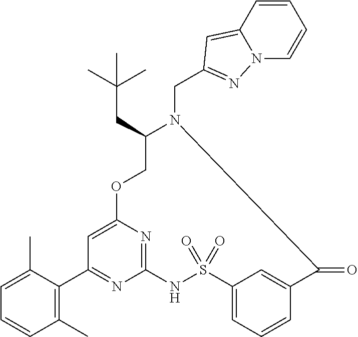 [SMILES: Cc1cccc(C)c1-c1cc2nc(n1)NS(=O)(=O)c1cccc(c1)C(=O)N(Cc1cc3ccccn3n1)[C@H](CC(C)(C)C)CO2] | | | + |
| 758 | 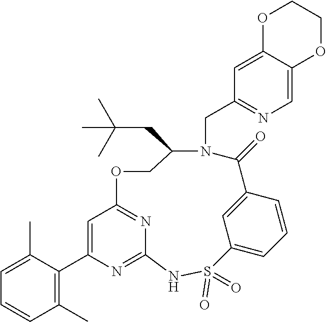 [SMILES: Cc1cccc(C)c1-c1cc2nc(n1)NS(=O)(=O)c1cccc(c1)C(=O)N(Cc1cc3c(cn1)OCCO3)[C@H](CC(C)(C)C)CO2] | | | +++ |
| 742 | 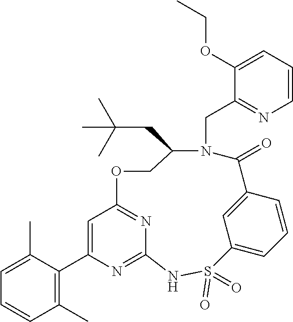 [SMILES: CCOc1cccnc1CN1C(=O)c2cccc(c2)S(=O)(=O)Nc2nc(cc(-c3c(C)cccc3C)n2)OC[C@H]1CC(C)(C)C] | +++ | +++ | |
| 741 | 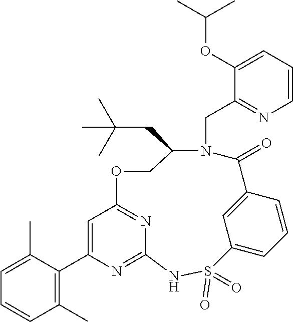 [SMILES: Cc1cccc(C)c1-c1cc2nc(n1)NS(=O)(=O)c1cccc(c1)C(=O)N(Cc1ncccc1OC(C)C)[C@H](CC(C)(C)C)CO2] | +++ | +++ | |
| 820 | 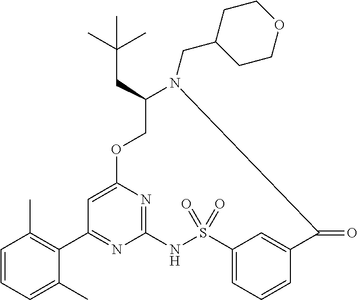 [SMILES: Cc1cccc(C)c1-c1cc2nc(n1)NS(=O)(=O)c1cccc(c1)C(=O)N(CC1CCOCC1)[C@H](CC(C)(C)C)CO2] | +++ | +++ | |
| 818 | 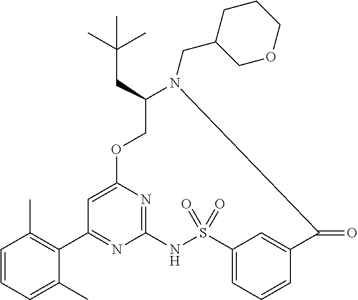 [SMILES: Cc1cccc(C)c1-c1cc2nc(n1)NS(=O)(=O)c1cccc(c1)C(=O)N(CC1CCCOC1)[C@H](CC(C)(C)C)CO2] | +++ | +++ | |
| 819 | 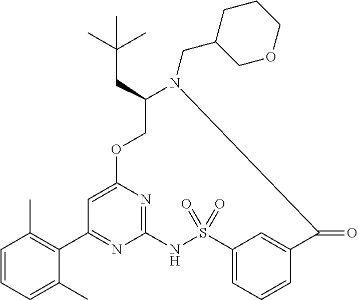 [SMILES: Cc1cccc(C)c1-c1cc2nc(n1)NS(=O)(=O)c1cccc(c1)C(=O)N(CC1CCCOC1)[C@H](CC(C)(C)C)CO2] | +++ | +++ | |
| 902 | 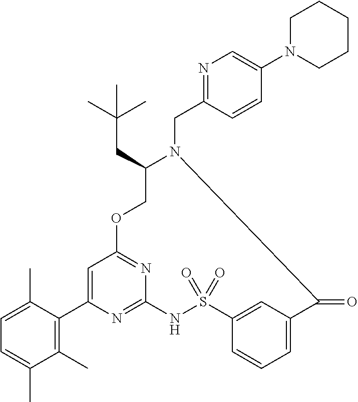 [SMILES: Cc1ccc(C)c(-c2cc3nc(n2)NS(=O)(=O)c2cccc(c2)C(=O)N(Cc2ccc(N4CCCCC4)cn2)[C@H](CC(C)(C)C)CO3)c1C] | | + | |
| 59 | 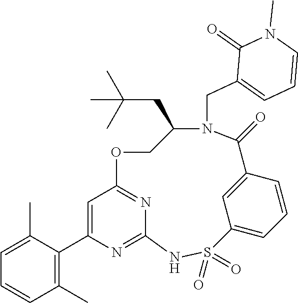 [SMILES: Cc1cccc(C)c1-c1cc2nc(n1)NS(=O)(=O)c1cccc(c1)C(=O)N(Cc1cccn(C)c1=O)[C@H](CC(C)(C)C)CO2] | +++ | +++ | |
| 1071 | 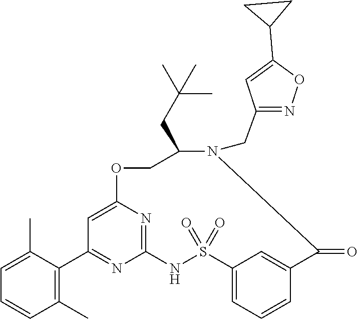 [SMILES: Cc1cccc(C)c1-c1cc2nc(n1)NS(=O)(=O)c1cccc(c1)C(=O)N(Cc1cc(C3CC3)on1)[C@H](CC(C)(C)C)CO2] | +++ | +++ | |
| 137 | 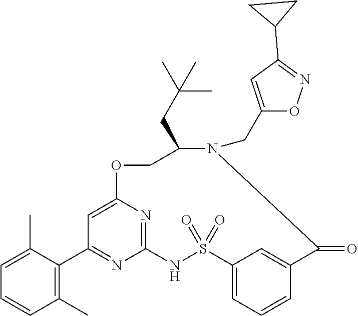 [SMILES: Cc1cccc(C)c1-c1cc2nc(n1)NS(=O)(=O)c1cccc(c1)C(=O)N(Cc1cc(C3CC3)no1)[C@H](CC(C)(C)C)CO2] | +++ | +++ | |
| 817 | 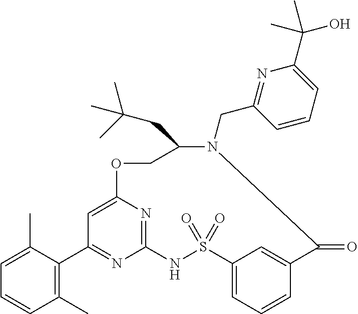 [SMILES: Cc1cccc(C)c1-c1cc2nc(n1)NS(=O)(=O)c1cccc(c1)C(=O)N(Cc1cccc(C(C)(C)O)n1)[C@H](CC(C)(C)C)CO2] | +++ | +++ | |
| 816 | 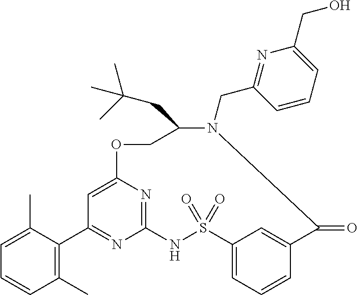 [SMILES: Cc1cccc(C)c1-c1cc2nc(n1)NS(=O)(=O)c1cccc(c1)C(=O)N(Cc1cccc(CO)n1)[C@H](CC(C)(C)C)CO2] | +++ | +++ | |
| 19 | 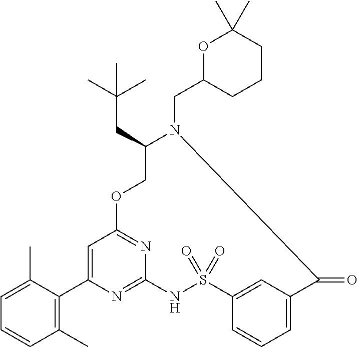 [SMILES: Cc1cccc(C)c1-c1cc2nc(n1)NS(=O)(=O)c1cccc(c1)C(=O)N(CC1CCCC(C)(C)O1)[C@H](CC(C)(C)C)CO2] | | | +++ |
| 20 | 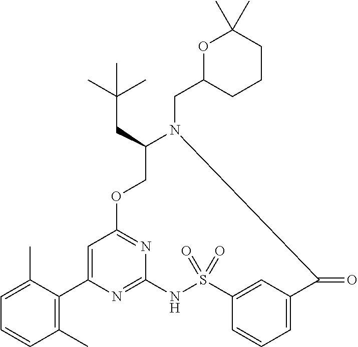 [SMILES: Cc1cccc(C)c1-c1cc2nc(n1)NS(=O)(=O)c1cccc(c1)C(=O)N(CC1CCCC(C)(C)O1)[C@H](CC(C)(C)C)CO2] | | | +++ |
| 21 | 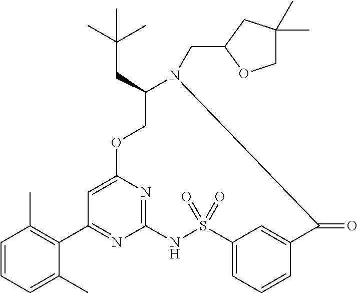 [SMILES: Cc1cccc(C)c1-c1cc2nc(n1)NS(=O)(=O)c1cccc(c1)C(=O)N(CC1CC(C)(C)CO1)[C@H](CC(C)(C)C)CO2] | | | +++ |
| 22 | 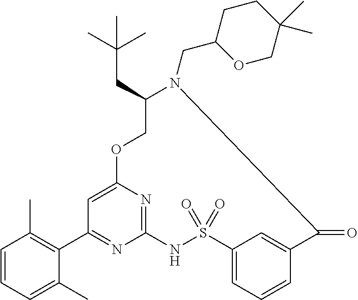 [SMILES: Cc1cccc(C)c1-c1cc2nc(n1)NS(=O)(=O)c1cccc(c1)C(=O)N(CC1CCC(C)(C)CO1)[C@H](CC(C)(C)C)CO2] | +++ | +++ | |
| 23 | 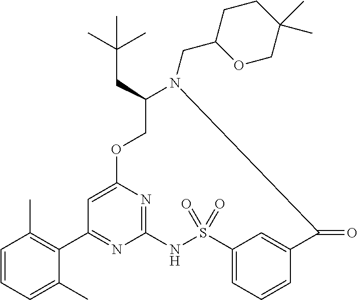 [SMILES: Cc1cccc(C)c1-c1cc2nc(n1)NS(=O)(=O)c1cccc(c1)C(=O)N(CC1CCC(C)(C)CO1)[C@H](CC(C)(C)C)CO2] | +++ | +++ | |
| 740 | 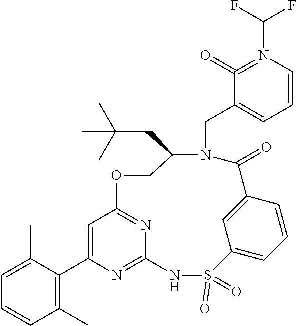 | | | +++ |
| 738 | 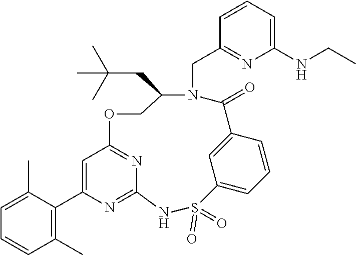 [SMILES: CCNc1cccc(CN2C(=O)c3cccc(c3)S(=O)(=O)Nc3nc(cc(-c4c(C)cccc4C)n3)OC[C@H]2CC(C)(C)C)n1] | +++ | +++ | |
| 739 | 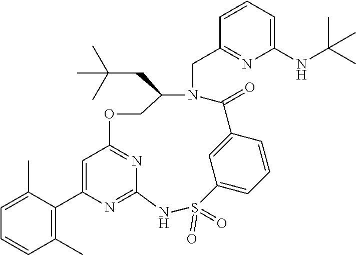 [SMILES: Cc1cccc(C)c1-c1cc2nc(n1)NS(=O)(=O)c1cccc(c1)C(=O)N(Cc1cccc(NC(C)(C)C)n1)[C@H](CC(C)(C)C)CO2] | +++ | +++ | |
| 736 | 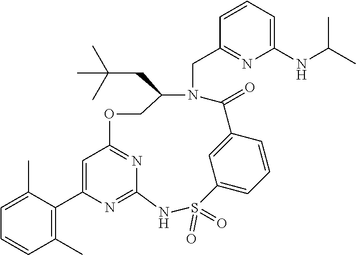 [SMILES: Cc1cccc(C)c1-c1cc2nc(n1)NS(=O)(=O)c1cccc(c1)C(=O)N(Cc1cccc(NC(C)C)n1)[C@H](CC(C)(C)C)CO2] | +++ | +++ | |
| 737 | 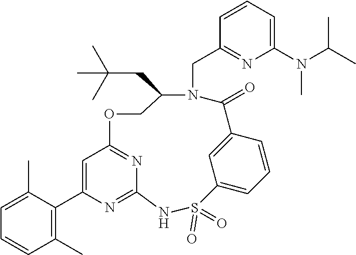 [SMILES: Cc1cccc(C)c1-c1cc2nc(n1)NS(=O)(=O)c1cccc(c1)C(=O)N(Cc1cccc(N(C)C(C)C)n1)[C@H](CC(C)(C)C)CO2] | +++ | +++ | |
| 735 | 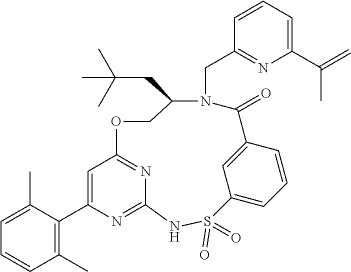 [SMILES: C=C(C)c1cccc(CN2C(=O)c3cccc(c3)S(=O)(=O)Nc3nc(cc(-c4c(C)cccc4C)n3)OC[C@H]2CC(C)(C)C)n1] | +++ | +++ | +++ |
| 733 | 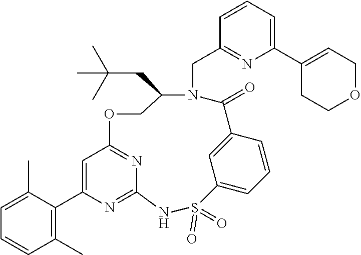 [SMILES: Cc1cccc(C)c1-c1cc2nc(n1)NS(=O)(=O)c1cccc(c1)C(=O)N(Cc1cccc(C3=CCOCC3)n1)[C@H](CC(C)(C)C)CO2] | +++ | +++ | +++ |
| 734 | 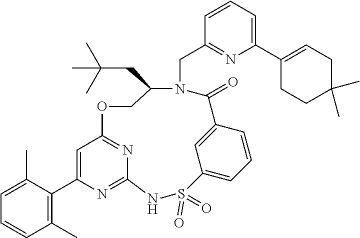 [SMILES: Cc1cccc(C)c1-c1cc2nc(n1)NS(=O)(=O)c1cccc(c1)C(=O)N(Cc1cccc(C3=CCC(C)(C)CC3)n1)[C@H](CC(C)(C)C)CO2] | | | +++ |
| 731 | 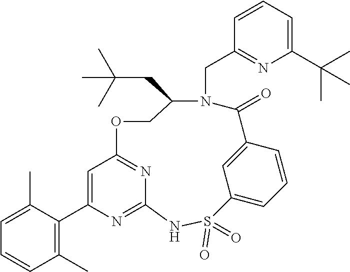 [SMILES: Cc1cccc(C)c1-c1cc2nc(n1)NS(=O)(=O)c1cccc(c1)C(=O)N(Cc1cccc(C(C)(C)C)n1)[C@H](CC(C)(C)C)CO2] | +++ | +++ | |
| 730 | 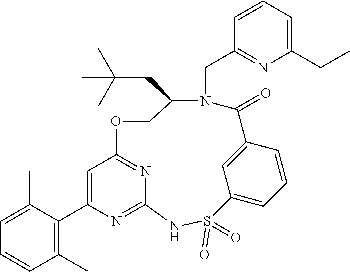 [SMILES: CCc1cccc(CN2C(=O)c3cccc(c3)S(=O)(=O)Nc3nc(cc(-c4c(C)cccc4C)n3)OC[C@H]2CC(C)(C)C)n1] | +++ | +++ | |
| 729 | 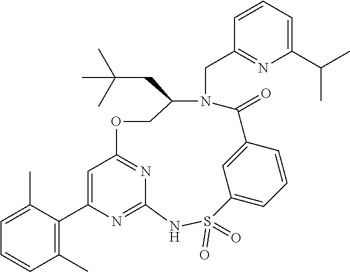 [SMILES: Cc1cccc(C)c1-c1cc2nc(n1)NS(=O)(=O)c1cccc(c1)C(=O)N(Cc1cccc(C(C)C)n1)[C@H](CC(C)(C)C)CO2] | +++ | +++ | |
| 732 | 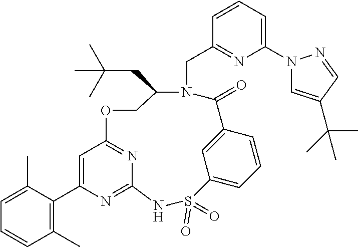 [SMILES: Cc1cccc(C)c1-c1cc2nc(n1)NS(=O)(=O)c1cccc(c1)C(=O)N(Cc1cccc(-n3cc(C(C)(C)C)cn3)n1)[C@H](CC(C)(C)C)CO2] | +++ | +++ | |
| 333 | 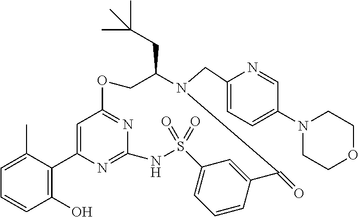 [SMILES: Cc1cccc(O)c1-c1cc2nc(n1)NS(=O)(=O)c1cccc(c1)C(=O)N(Cc1ccc(N3CCOCC3)cn1)[C@H](CC(C)(C)C)CO2] | | | ++ |
| 24 | 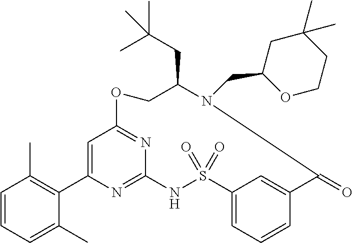 [SMILES: Cc1cccc(C)c1-c1cc2nc(n1)NS(=O)(=O)c1cccc(c1)C(=O)N(C[C@H]1CC(C)(C)CCO1)[C@H](CC(C)(C)C)CO2] | | | +++ |
| 25 | 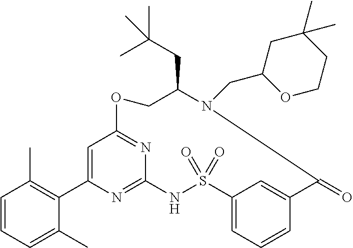 [SMILES: Cc1cccc(C)c1-c1cc2nc(n1)NS(=O)(=O)c1cccc(c1)C(=O)N(CC1CC(C)(C)CCO1)[C@H](CC(C)(C)C)CO2] | | | +++ |
| 26 | 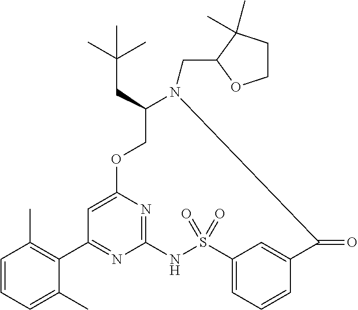 [SMILES: Cc1cccc(C)c1-c1cc2nc(n1)NS(=O)(=O)c1cccc(c1)C(=O)N(CC1OCCC1(C)C)[C@H](CC(C)(C)C)CO2] | | | +++ |
| 27 | 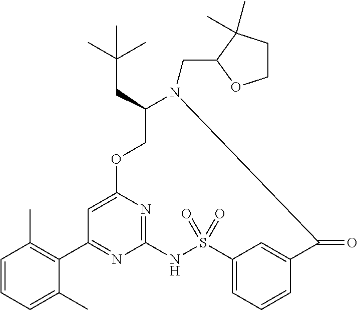 [SMILES: Cc1cccc(C)c1-c1cc2nc(n1)NS(=O)(=O)c1cccc(c1)C(=O)N(CC1OCCC1(C)C)[C@H](CC(C)(C)C)CO2] | | | +++ |
| 814 | 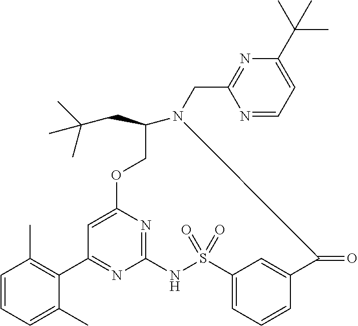 [SMILES: Cc1cccc(C)c1-c1cc2nc(n1)NS(=O)(=O)c1cccc(c1)C(=O)N(Cc1nccc(C(C)(C)C)n1)[C@H](CC(C)(C)C)CO2] | +++ | +++ | |
| 815 | 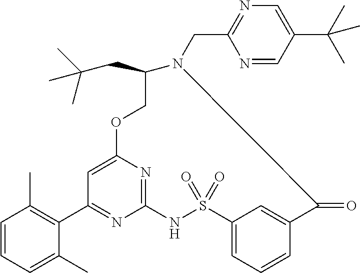 [SMILES: Cc1cccc(C)c1-c1cc2nc(n1)NS(=O)(=O)c1cccc(c1)C(=O)N(Cc1ncc(C(C)(C)C)cn1)[C@H](CC(C)(C)C)CO2] | +++ | +++ | |
| 28 | 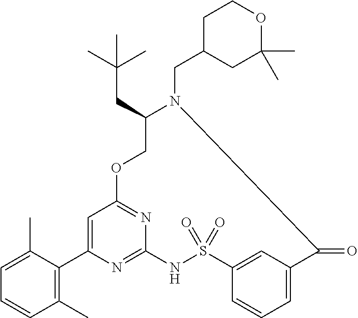 [SMILES: Cc1cccc(C)c1-c1cc2nc(n1)NS(=O)(=O)c1cccc(c1)C(=O)N(CC1CCOC(C)(C)C1)[C@H](CC(C)(C)C)CO2] | | | +++ |
| 29 | 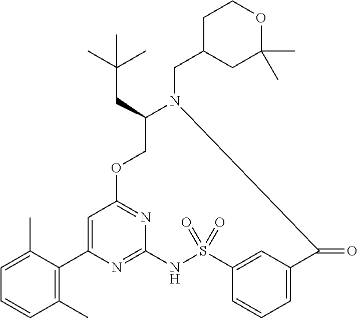 [SMILES: Cc1cccc(C)c1-c1cc2nc(n1)NS(=O)(=O)c1cccc(c1)C(=O)N(CC1CCOC(C)(C)C1)[C@H](CC(C)(C)C)CO2] | | | +++ |
| 30 | 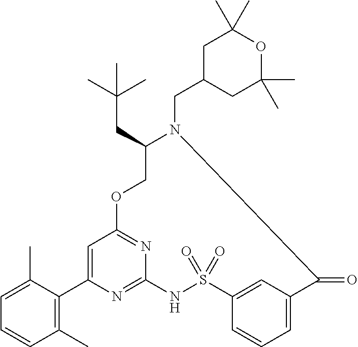 [SMILES: Cc1cccc(C)c1-c1cc2nc(n1)NS(=O)(=O)c1cccc(c1)C(=O)N(CC1CC(C)(C)OC(C)(C)C1)[C@H](CC(C)(C)C)CO2] | | | +++ |
| 900 | 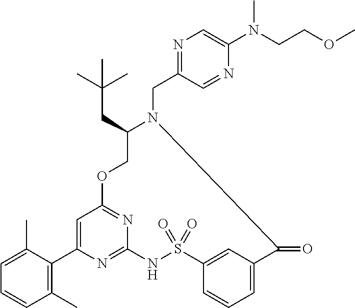 [SMILES: COCCN(C)c1cnc(CN2C(=O)c3cccc(c3)S(=O)(=O)Nc3nc(cc(-c4c(C)cccc4C)n3)OC[C@H]2CC(C)(C)C)cn1] | ++ | +++ | |
| 897 | 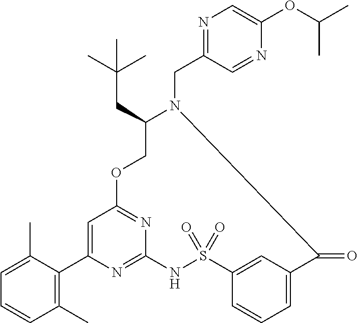 [SMILES: Cc1cccc(C)c1-c1cc2nc(n1)NS(=O)(=O)c1cccc(c1)C(=O)N(Cc1cnc(OC(C)C)cn1)[C@H](CC(C)(C)C)CO2] | +++ | +++ | |
| 31 | 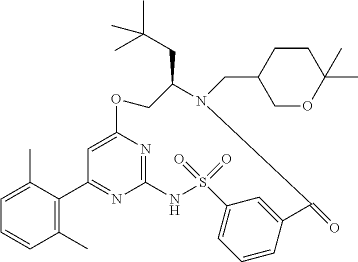 [SMILES: Cc1cccc(C)c1-c1cc2nc(n1)NS(=O)(=O)c1cccc(c1)C(=O)N(CC1CCC(C)(C)OC1)[C@H](CC(C)(C)C)CO2] | | | +++ |
| 32 | 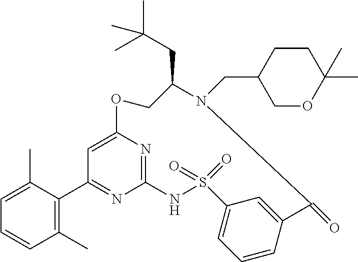 [SMILES: Cc1cccc(C)c1-c1cc2nc(n1)NS(=O)(=O)c1cccc(c1)C(=O)N(CC1CCC(C)(C)OC1)[C@H](CC(C)(C)C)CO2] | | | +++ |
| 727 | 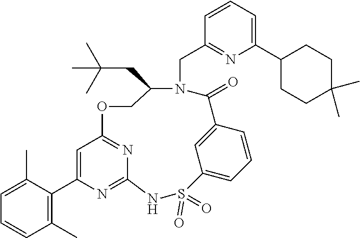 [SMILES: Cc1cccc(C)c1-c1cc2nc(n1)NS(=O)(=O)c1cccc(c1)C(=O)N(Cc1cccc(C3CCC(C)(C)CC3)n1)[C@H](CC(C)(C)C)CO2] | +++ | +++ | |
| 728 | 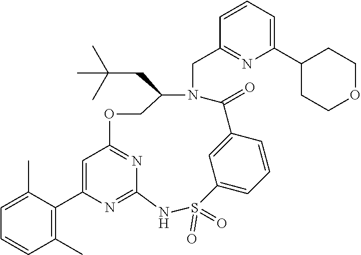 [SMILES: Cc1cccc(C)c1-c1cc2nc(n1)NS(=O)(=O)c1cccc(c1)C(=O)N(Cc1cccc(C3CCOCC3)n1)[C@H](CC(C)(C)C)CO2] | +++ | +++ | |
| 813 | 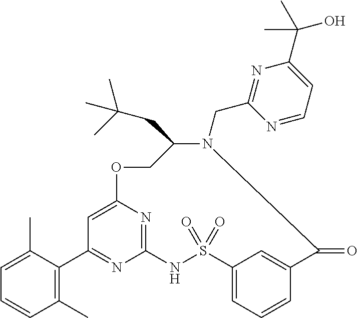 [SMILES: Cc1cccc(C)c1-c1cc2nc(n1)NS(=O)(=O)c1cccc(c1)C(=O)N(Cc1nccc(C(C)(C)O)n1)[C@H](CC(C)(C)C)CO2] | +++ | +++ | |
| 812 | 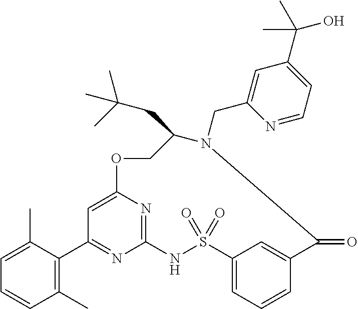 [SMILES: Cc1cccc(C)c1-c1cc2nc(n1)NS(=O)(=O)c1cccc(c1)C(=O)N(Cc1cc(C(C)(C)O)ccn1)[C@H](CC(C)(C)C)CO2] | +++ | +++ | |
| 899 | 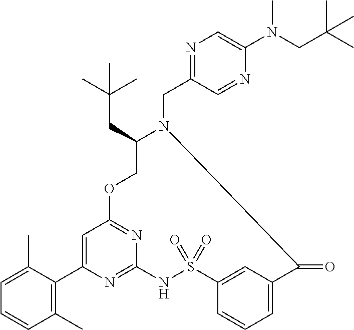 [SMILES: Cc1cccc(C)c1-c1cc2nc(n1)NS(=O)(=O)c1cccc(c1)C(=O)N(Cc1cnc(N(C)CC(C)(C)C)cn1)[C@H](CC(C)(C)C)CO2] | +++ | +++ | |
| 898 | 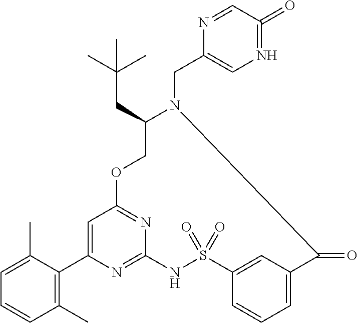 | | | ++ |
| 121 | 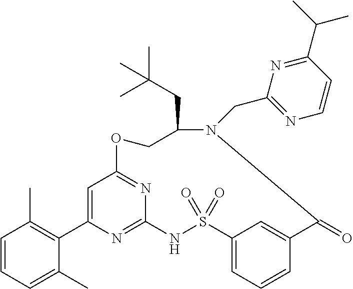 [SMILES: Cc1cccc(C)c1-c1cc2nc(n1)NS(=O)(=O)c1cccc(c1)C(=O)N(Cc1nccc(C(C)C)n1)[C@H](CC(C)(C)C)CO2] | +++ | +++ | |
| 657 | 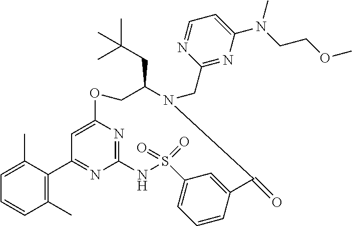 [SMILES: COCCN(C)c1ccnc(CN2C(=O)c3cccc(c3)S(=O)(=O)Nc3nc(cc(-c4c(C)cccc4C)n3)OC[C@H]2CC(C)(C)C)n1] | +++ | +++ | |
| 656 | 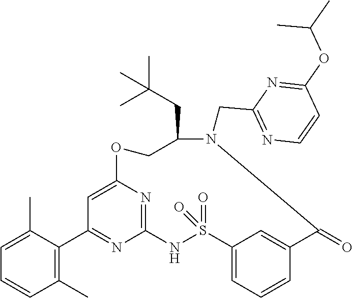 [SMILES: Cc1cccc(C)c1-c1cc2nc(n1)NS(=O)(=O)c1cccc(c1)C(=O)N(Cc1nccc(OC(C)C)n1)[C@H](CC(C)(C)C)CO2] | +++ | +++ | |
| 654 | 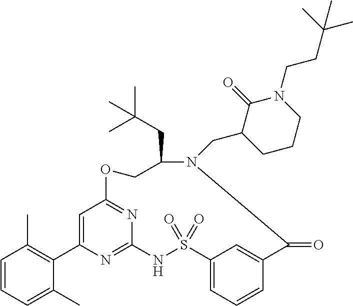 [SMILES: Cc1cccc(C)c1-c1cc2nc(n1)NS(=O)(=O)c1cccc(c1)C(=O)N(CC1CCCN(CCC(C)(C)C)C1=O)[C@H](CC(C)(C)C)CO2] | +++ | +++ | |
| 655 | 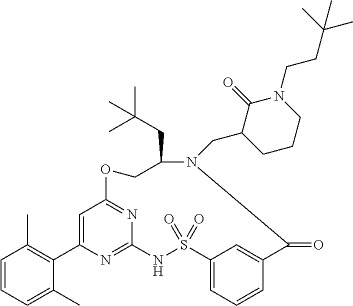 [SMILES: Cc1cccc(C)c1-c1cc2nc(n1)NS(=O)(=O)c1cccc(c1)C(=O)N(CC1CCCN(CCC(C)(C)C)C1=O)[C@H](CC(C)(C)C)CO2] | +++ | +++ | |
| 811 | 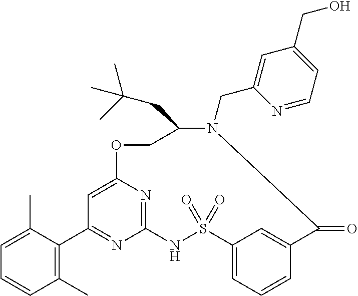 [SMILES: Cc1cccc(C)c1-c1cc2nc(n1)NS(=O)(=O)c1cccc(c1)C(=O)N(Cc1cc(CO)ccn1)[C@H](CC(C)(C)C)CO2] | +++ | +++ | |
| 33 | 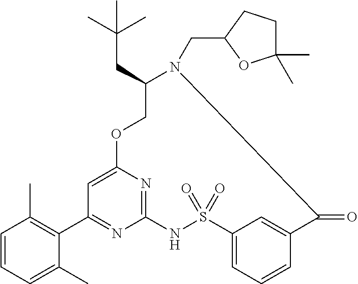 [SMILES: Cc1cccc(C)c1-c1cc2nc(n1)NS(=O)(=O)c1cccc(c1)C(=O)N(CC1CCC(C)(C)O1)[C@H](CC(C)(C)C)CO2] | | | +++ |
| 34 | 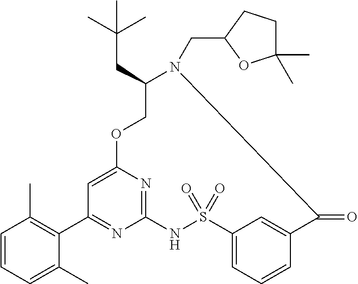 [SMILES: Cc1cccc(C)c1-c1cc2nc(n1)NS(=O)(=O)c1cccc(c1)C(=O)N(CC1CCC(C)(C)O1)[C@H](CC(C)(C)C)CO2] | +++ | +++ | +++ |
| 726 | 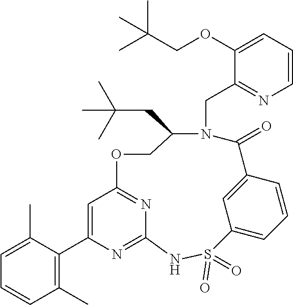 [SMILES: Cc1cccc(C)c1-c1cc2nc(n1)NS(=O)(=O)c1cccc(c1)C(=O)N(Cc1ncccc1OCC(C)(C)C)[C@H](CC(C)(C)C)CO2] | | | |
| 725 | 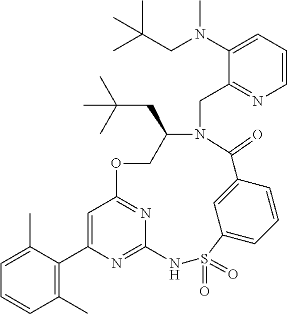 [SMILES: Cc1cccc(C)c1-c1cc2nc(n1)NS(=O)(=O)c1cccc(c1)C(=O)N(Cc1ncccc1N(C)CC(C)(C)C)[C@H](CC(C)(C)C)CO2] | +++ | +++ | |
| 652 | 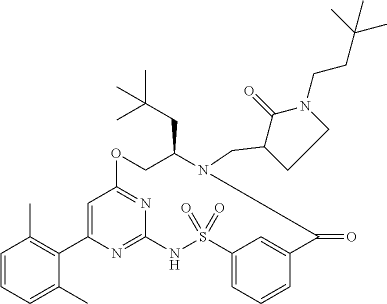 [SMILES: Cc1cccc(C)c1-c1cc2nc(n1)NS(=O)(=O)c1cccc(c1)C(=O)N(CC1CCN(CCC(C)(C)C)C1=O)[C@H](CC(C)(C)C)CO2] | +++ | +++ | +++ |
| 653 | 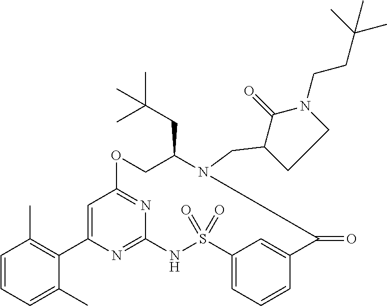 [SMILES: Cc1cccc(C)c1-c1cc2nc(n1)NS(=O)(=O)c1cccc(c1)C(=O)N(CC1CCN(CCC(C)(C)C)C1=O)[C@H](CC(C)(C)C)CO2] | +++ | +++ | +++ |
| 651 | 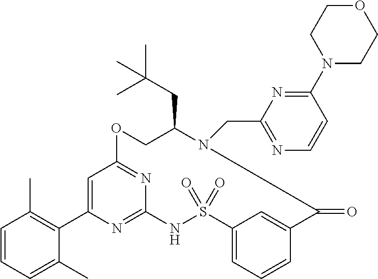 [SMILES: Cc1cccc(C)c1-c1cc2nc(n1)NS(=O)(=O)c1cccc(c1)C(=O)N(Cc1nccc(N3CCOCC3)n1)[C@H](CC(C)(C)C)CO2] | +++ | +++ | |
| 650 | 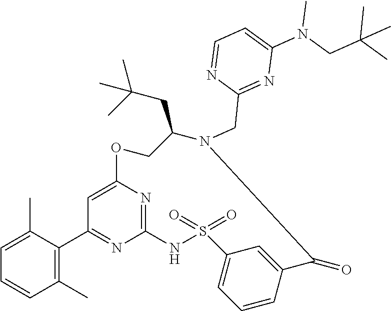 [SMILES: Cc1cccc(C)c1-c1cc2nc(n1)NS(=O)(=O)c1cccc(c1)C(=O)N(Cc1nccc(N(C)CC(C)(C)C)n1)[C@H](CC(C)(C)C)CO2] | +++ | +++ | |
| 649 | 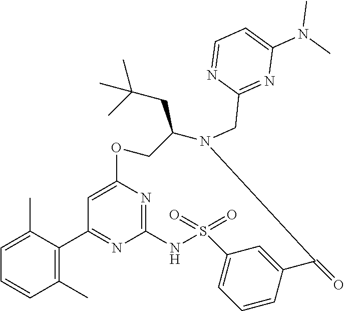 [SMILES: Cc1cccc(C)c1-c1cc2nc(n1)NS(=O)(=O)c1cccc(c1)C(=O)N(Cc1nccc(N(C)C)n1)[C@H](CC(C)(C)C)CO2] | +++ | +++ | |
| 648 | 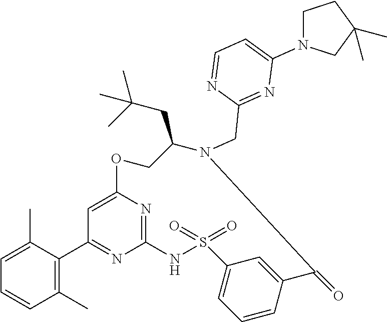 [SMILES: Cc1cccc(C)c1-c1cc2nc(n1)NS(=O)(=O)c1cccc(c1)C(=O)N(Cc1nccc(N3CCC(C)(C)C3)n1)[C@H](CC(C)(C)C)CO2] | +++ | +++ | |
| 647 | 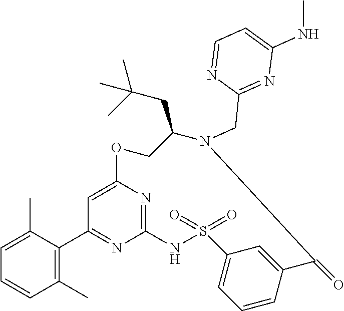 [SMILES: CNc1ccnc(CN2C(=O)c3cccc(c3)S(=O)(=O)Nc3nc(cc(-c4c(C)cccc4C)n3)OC[C@H]2CC(C)(C)C)n1] | +++ | +++ | |
| 896 | 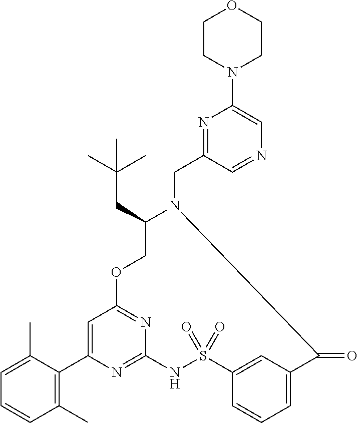 [SMILES: Cc1cccc(C)c1-c1cc2nc(n1)NS(=O)(=O)c1cccc(c1)C(=O)N(Cc1cncc(N3CCOCC3)n1)[C@H](CC(C)(C)C)CO2] | +++ | +++ | |
| 895 | 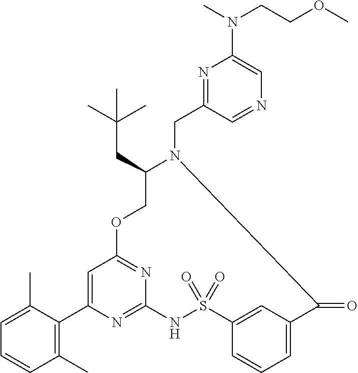 [SMILES: COCCN(C)c1cncc(CN2C(=O)c3cccc(c3)S(=O)(=O)Nc3nc(cc(-c4c(C)cccc4C)n3)OC[C@H]2CC(C)(C)C)n1] | +++ | +++ | |
| 894 | 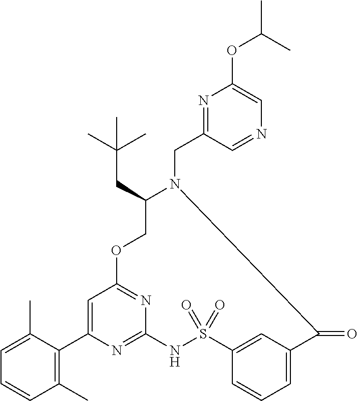 [SMILES: Cc1cccc(C)c1-c1cc2nc(n1)NS(=O)(=O)c1cccc(c1)C(=O)N(Cc1cncc(OC(C)C)n1)[C@H](CC(C)(C)C)CO2] | +++ | +++ | |
| 646 | 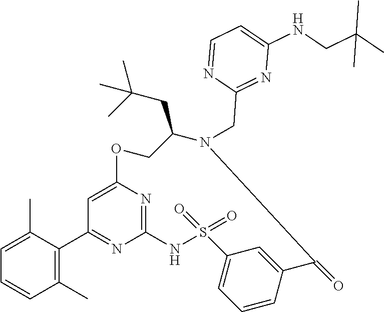 [SMILES: Cc1cccc(C)c1-c1cc2nc(n1)NS(=O)(=O)c1cccc(c1)C(=O)N(Cc1nccc(NCC(C)(C)C)n1)[C@H](CC(C)(C)C)CO2] | +++ | +++ | |
| 35 | 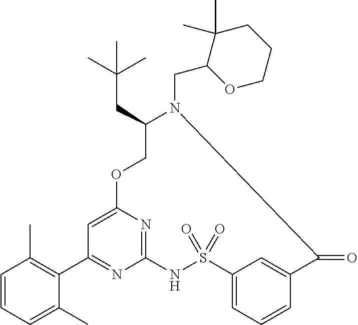 [SMILES: Cc1cccc(C)c1-c1cc2nc(n1)NS(=O)(=O)c1cccc(c1)C(=O)N(CC1OCCCC1(C)C)[C@H](CC(C)(C)C)CO2] | | | +++ |
| 36 | 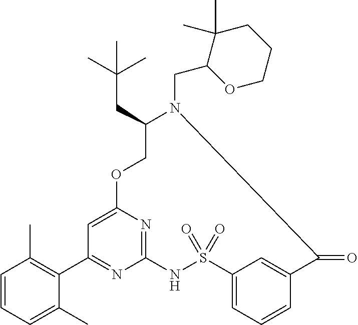 [SMILES: Cc1cccc(C)c1-c1cc2nc(n1)NS(=O)(=O)c1cccc(c1)C(=O)N(CC1OCCCC1(C)C)[C@H](CC(C)(C)C)CO2] | | | +++ |
| 719 | 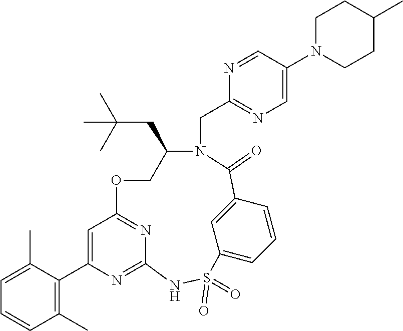 [SMILES: Cc1cccc(C)c1-c1cc2nc(n1)NS(=O)(=O)c1cccc(c1)C(=O)N(Cc1ncc(N3CCC(C)CC3)cn1)[C@H](CC(C)(C)C)CO2] | +++ | +++ | |
| 718 | 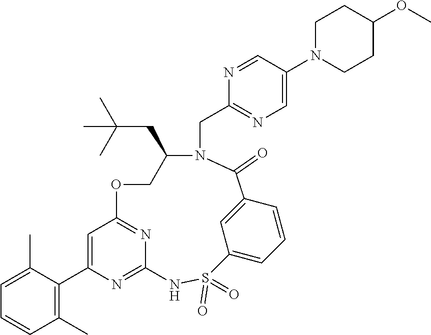 [SMILES: COC1CCN(c2cnc(CN3C(=O)c4cccc(c4)S(=O)(=O)Nc4nc(cc(-c5c(C)cccc5C)n4)OC[C@H]3CC(C)(C)C)nc2)CC1] | +++ | +++ | |
| 717 | 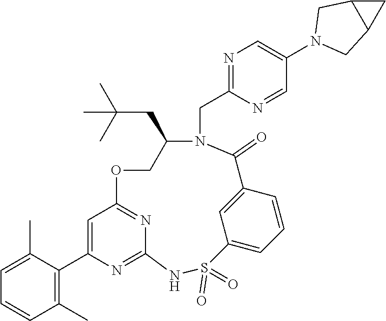 [SMILES: Cc1cccc(C)c1-c1cc2nc(n1)NS(=O)(=O)c1cccc(c1)C(=O)N(Cc1ncc(N3CC4CC4C3)cn1)[C@H](CC(C)(C)C)CO2] | ++ | +++ | |
| 117 | 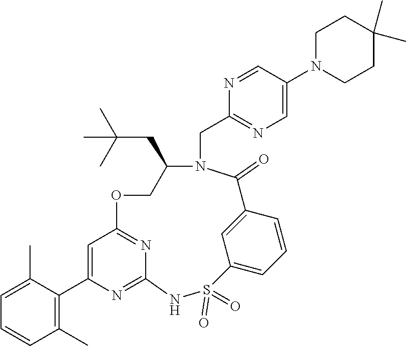 [SMILES: Cc1cccc(C)c1-c1cc2nc(n1)NS(=O)(=O)c1cccc(c1)C(=O)N(Cc1ncc(N3CCC(C)(C)CC3)cn1)[C@H](CC(C)(C)C)CO2] | +++ | +++ | |
| 716 | 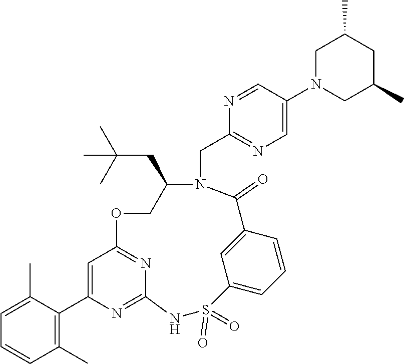 [SMILES: Cc1cccc(C)c1-c1cc2nc(n1)NS(=O)(=O)c1cccc(c1)C(=O)N(Cc1ncc(N3C[C@H](C)C[C@@H](C)C3)cn1)[C@H](CC(C)(C)C)CO2] | + | +++ | |
| 893 | 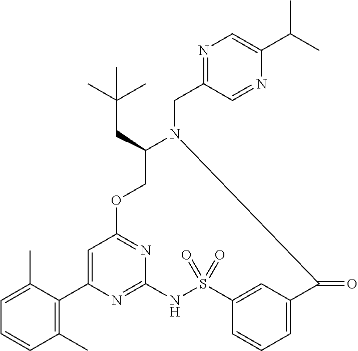 [SMILES: Cc1cccc(C)c1-c1cc2nc(n1)NS(=O)(=O)c1cccc(c1)C(=O)N(Cc1cnc(C(C)C)cn1)[C@H](CC(C)(C)C)CO2] | +++ | +++ | |
| 892 | 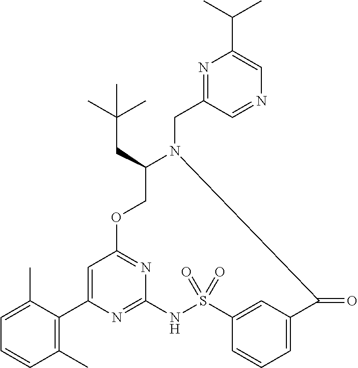 [SMILES: Cc1cccc(C)c1-c1cc2nc(n1)NS(=O)(=O)c1cccc(c1)C(=O)N(Cc1cncc(C(C)C)n1)[C@H](CC(C)(C)C)CO2] | +++ | +++ | |
| 141 | 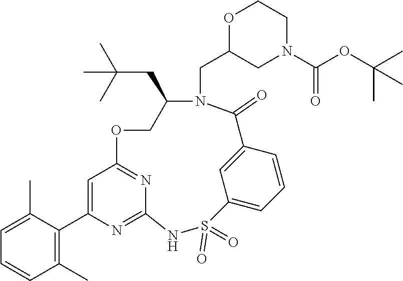 [SMILES: Cc1cccc(C)c1-c1cc2nc(n1)NS(=O)(=O)c1cccc(c1)C(=O)N(CC1CN(C(=O)OC(C)(C)C)CCO1)[C@H](CC(C)(C)C)CO2] | +++ | +++ | |
| 723 | 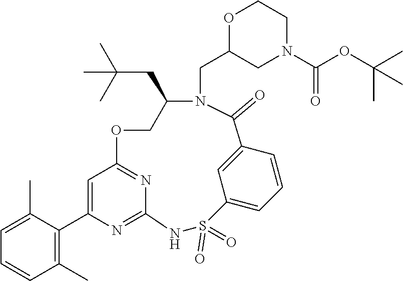 [SMILES: Cc1cccc(C)c1-c1cc2nc(n1)NS(=O)(=O)c1cccc(c1)C(=O)N(CC1CN(C(=O)OC(C)(C)C)CCO1)[C@H](CC(C)(C)C)CO2] | +++ | +++ | |
| 721 | 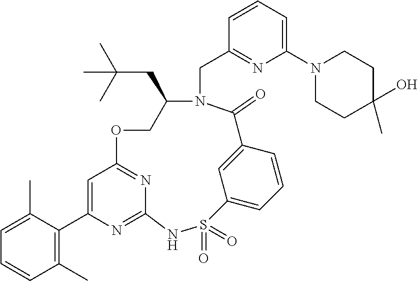 [SMILES: Cc1cccc(C)c1-c1cc2nc(n1)NS(=O)(=O)c1cccc(c1)C(=O)N(Cc1cccc(N3CCC(C)(O)CC3)n1)[C@H](CC(C)(C)C)CO2] | +++ | +++ | |
| 722 | 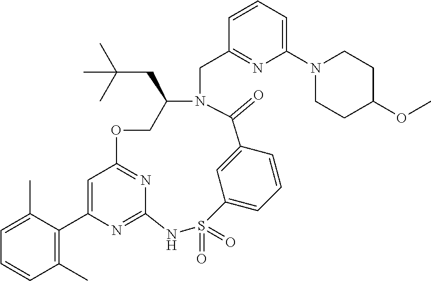 [SMILES: COC1CCN(c2cccc(CN3C(=O)c4cccc(c4)S(=O)(=O)Nc4nc(cc(-c5c(C)cccc5C)n4)OC[C@H]3CC(C)(C)C)n2)CC1] | +++ | +++ | |
| 720 | 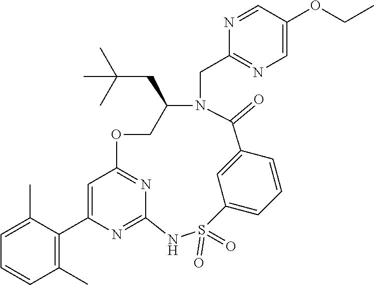 [SMILES: CCOc1cnc(CN2C(=O)c3cccc(c3)S(=O)(=O)Nc3nc(cc(-c4c(C)cccc4C)n3)OC[C@H]2CC(C)(C)C)nc1] | +++ | +++ | |
| 118 | 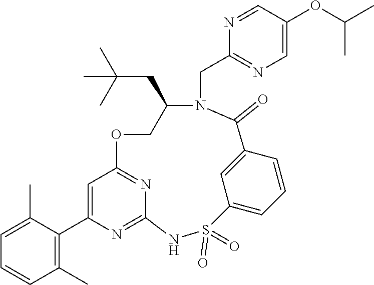 [SMILES: Cc1cccc(C)c1-c1cc2nc(n1)NS(=O)(=O)c1cccc(c1)C(=O)N(Cc1ncc(OC(C)C)cn1)[C@H](CC(C)(C)C)CO2] | +++ | +++ | |
| 645 | 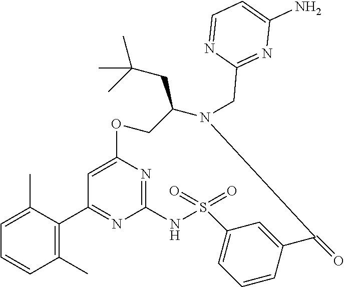 [SMILES: Cc1cccc(C)c1-c1cc2nc(n1)NS(=O)(=O)c1cccc(c1)C(=O)N(Cc1nccc(N)n1)[C@H](CC(C)(C)C)CO2] | +++ | +++ | |
| 810 | 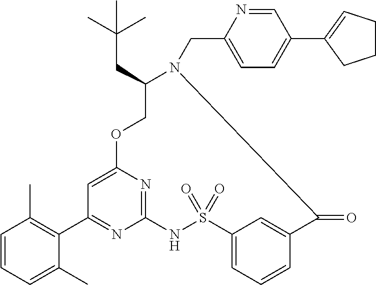 [SMILES: Cc1cccc(C)c1-c1cc2nc(n1)NS(=O)(=O)c1cccc(c1)C(=O)N(Cc1ccc(C3=CCCC3)cn1)[C@H](CC(C)(C)C)CO2] | +++ | +++ | |
| 809 | 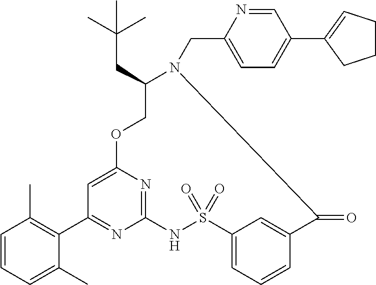 [SMILES: Cc1cccc(C)c1-c1cc2nc(n1)NS(=O)(=O)c1cccc(c1)C(=O)N(Cc1ccc(C3=CCCC3)cn1)[C@H](CC(C)(C)C)CO2] | +++ | +++ | |
| 714 | 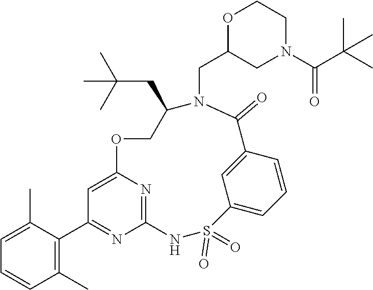 [SMILES: Cc1cccc(C)c1-c1cc2nc(n1)NS(=O)(=O)c1cccc(c1)C(=O)N(CC1CN(C(=O)C(C)(C)C)CCO1)[C@H](CC(C)(C)C)CO2] | +++ | +++ | |
| 142 | 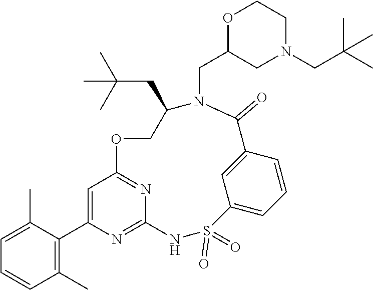 [SMILES: Cc1cccc(C)c1-c1cc2nc(n1)NS(=O)(=O)c1cccc(c1)C(=O)N(CC1CN(CC(C)(C)C)CCO1)[C@H](CC(C)(C)C)CO2] | +++ | +++ | |
| 713 | 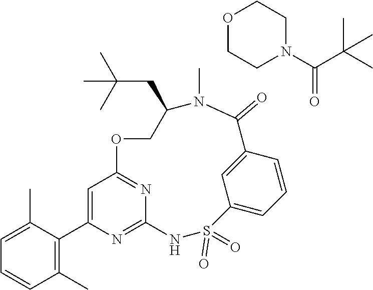 | +++ | +++ | |
| 712 | 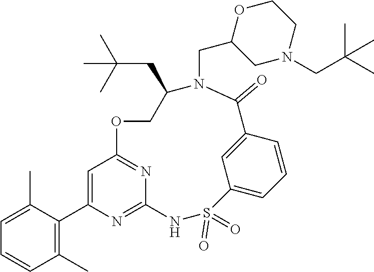 [SMILES: Cc1cccc(C)c1-c1cc2nc(n1)NS(=O)(=O)c1cccc(c1)C(=O)N(CC1CN(CC(C)(C)C)CCO1)[C@H](CC(C)(C)C)CO2] | +++ | +++ | |
| 891 | 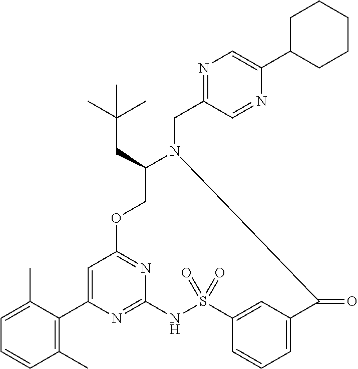 [SMILES: Cc1cccc(C)c1-c1cc2nc(n1)NS(=O)(=O)c1cccc(c1)C(=O)N(Cc1cnc(C3CCCCC3)cn1)[C@H](CC(C)(C)C)CO2] | ++ | +++ | |
| 890 | 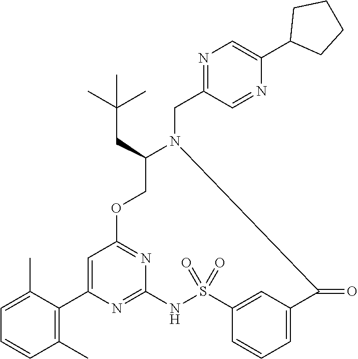 [SMILES: Cc1cccc(C)c1-c1cc2nc(n1)NS(=O)(=O)c1cccc(c1)C(=O)N(Cc1cnc(C3CCCC3)cn1)[C@H](CC(C)(C)C)CO2] | + | +++ | |
| 889 | 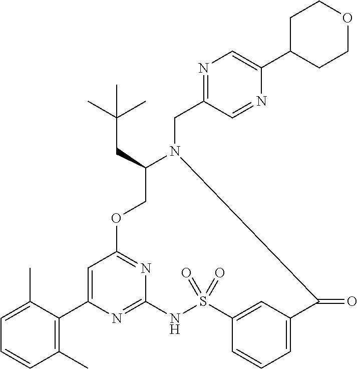 [SMILES: Cc1cccc(C)c1-c1cc2nc(n1)NS(=O)(=O)c1cccc(c1)C(=O)N(Cc1cnc(C3CCOCC3)cn1)[C@H](CC(C)(C)C)CO2] | ++ | +++ | |
| 903 | 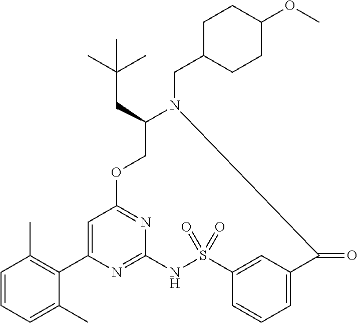 [SMILES: COC1CCC(CN2C(=O)c3cccc(c3)S(=O)(=O)Nc3nc(cc(-c4c(C)cccc4C)n3)OC[C@H]2CC(C)(C)C)CC1] | +++ | +++ | |
| 901 | 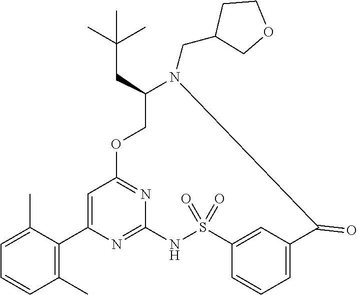 [SMILES: Cc1cccc(C)c1-c1cc2nc(n1)NS(=O)(=O)c1cccc(c1)C(=O)N(CC1CCOC1)[C@H](CC(C)(C)C)CO2] | | | +++ |
| 644 | 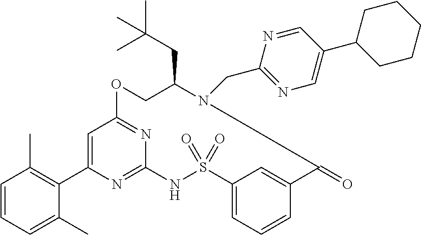 [SMILES: Cc1cccc(C)c1-c1cc2nc(n1)NS(=O)(=O)c1cccc(c1)C(=O)N(Cc1ncc(C3CCCCC3)cn1)[C@H](CC(C)(C)C)CO2] | ++ | +++ | |
| 724 | 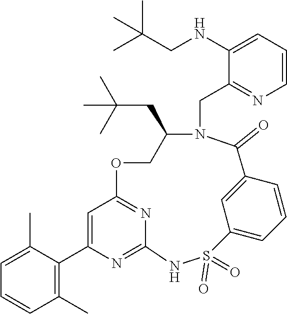 [SMILES: Cc1cccc(C)c1-c1cc2nc(n1)NS(=O)(=O)c1cccc(c1)C(=O)N(Cc1ncccc1NCC(C)(C)C)[C@H](CC(C)(C)C)CO2] | +++ | +++ | |
| 715 | 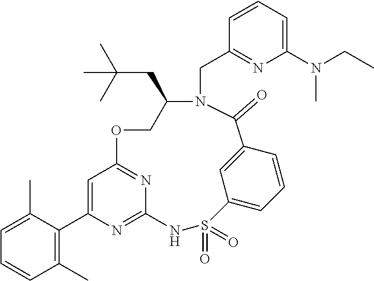 [SMILES: CCN(C)c1cccc(CN2C(=O)c3cccc(c3)S(=O)(=O)Nc3nc(cc(-c4c(C)cccc4C)n3)OC[C@H]2CC(C)(C)C)n1] | ++ | +++ | |
| 711 | 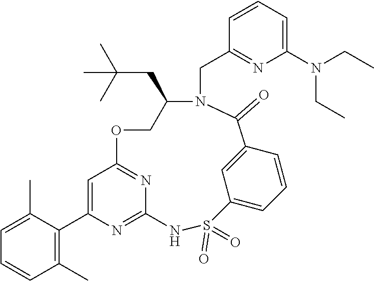 [SMILES: CCN(CC)c1cccc(CN2C(=O)c3cccc(c3)S(=O)(=O)Nc3nc(cc(-c4c(C)cccc4C)n3)OC[C@H]2CC(C)(C)C)n1] | ++ | +++ | |
| 888 | 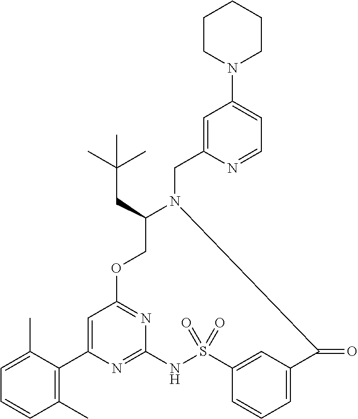 [SMILES: Cc1cccc(C)c1-c1cc2nc(n1)NS(=O)(=O)c1cccc(c1)C(=O)N(Cc1cc(N3CCCCC3)ccn1)[C@H](CC(C)(C)C)CO2] | +++ | +++ | |
| 887 | 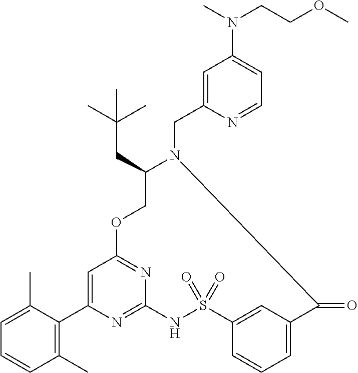 [SMILES: COCCN(C)c1ccnc(CN2C(=O)c3cccc(c3)S(=O)(=O)Nc3nc(cc(-c4c(C)cccc4C)n3)OC[C@H]2CC(C)(C)C)c1] | +++ | +++ | |
| 643 | 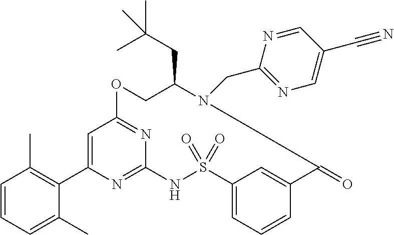 [SMILES: Cc1cccc(C)c1-c1cc2nc(n1)NS(=O)(=O)c1cccc(c1)C(=O)N(Cc1ncc(C#N)cn1)[C@H](CC(C)(C)C)CO2] | +++ | +++ | |
| 119 | 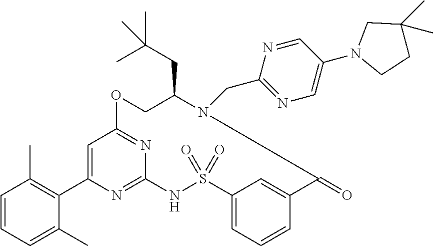 [SMILES: Cc1cccc(C)c1-c1cc2nc(n1)NS(=O)(=O)c1cccc(c1)C(=O)N(Cc1ncc(N3CCC(C)(C)C3)cn1)[C@H](CC(C)(C)C)CO2] | ++ | +++ | |
| 708 | 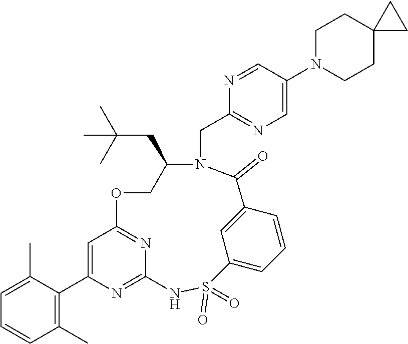 [SMILES: Cc1cccc(C)c1-c1cc2nc(n1)NS(=O)(=O)c1cccc(c1)C(=O)N(Cc1ncc(N3CCC4(CC3)CC4)cn1)[C@H](CC(C)(C)C)CO2] | ++ | +++ | |
| 886 | 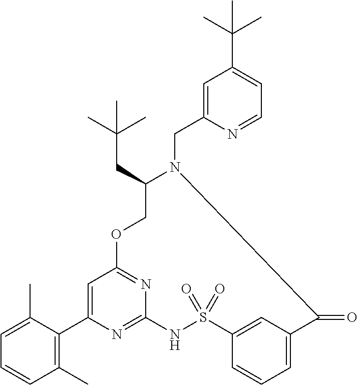 [SMILES: Cc1cccc(C)c1-c1cc2nc(n1)NS(=O)(=O)c1cccc(c1)C(=O)N(Cc1cc(C(C)(C)C)ccn1)[C@H](CC(C)(C)C)CO2] | +++ | +++ | |
| 885 | 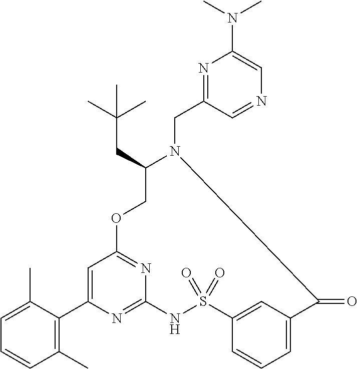 [SMILES: Cc1cccc(C)c1-c1cc2nc(n1)NS(=O)(=O)c1cccc(c1)C(=O)N(Cc1cncc(N(C)C)n1)[C@H](CC(C)(C)C)CO2] | +++ | +++ | |
| 62 | 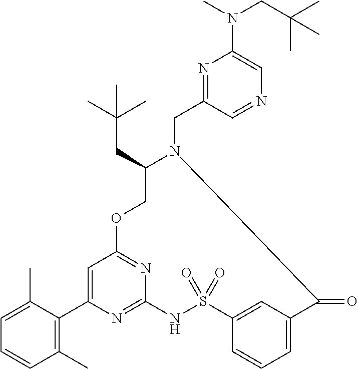 [SMILES: Cc1cccc(C)c1-c1cc2nc(n1)NS(=O)(=O)c1cccc(c1)C(=O)N(Cc1cncc(N(C)CC(C)(C)C)n1)[C@H](CC(C)(C)C)CO2] | +++ | +++ | |
| 884 | 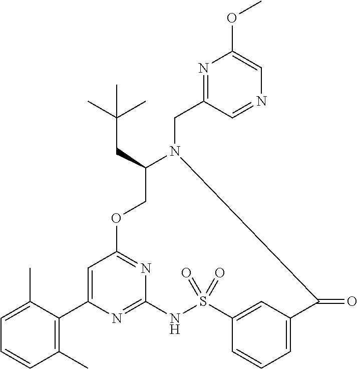 [SMILES: COc1cncc(CN2C(=O)c3cccc(c3)S(=O)(=O)Nc3nc(cc(-c4c(C)cccc4C)n3)OC[C@H]2CC(C)(C)C)n1] | +++ | +++ | |
| 122 | 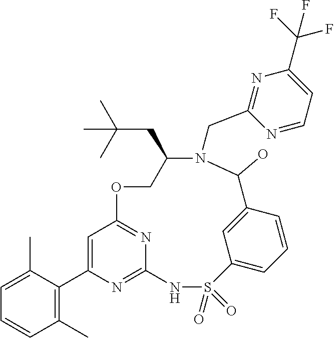 [SMILES: Cc1cccc(C)c1-c1cc2nc(n1)NS(=O)(=O)c1cccc(c1)C(O)N(Cc1nccc(C(F)(F)F)n1)[C@H](CC(C)(C)C)CO2] | +++ | +++ | |
| 1070 | 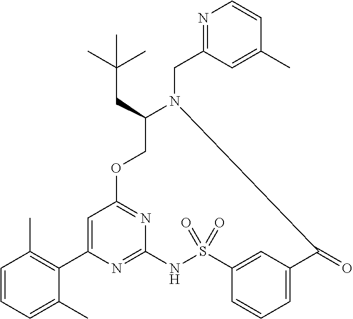 [SMILES: Cc1ccnc(CN2C(=O)c3cccc(c3)S(=O)(=O)Nc3nc(cc(-c4c(C)cccc4C)n3)OC[C@H]2CC(C)(C)C)c1] | +++ | +++ | |
| 1069 | 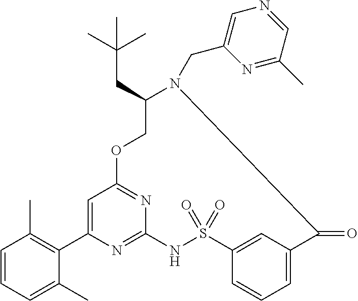 [SMILES: Cc1cncc(CN2C(=O)c3cccc(c3)S(=O)(=O)Nc3nc(cc(-c4c(C)cccc4C)n3)OC[C@H]2CC(C)(C)C)n1] | +++ | +++ | |
| 642 | 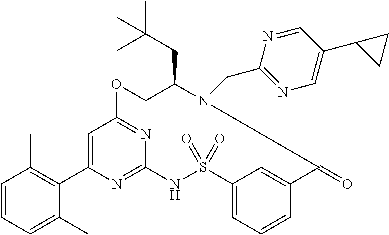 [SMILES: Cc1cccc(C)c1-c1cc2nc(n1)NS(=O)(=O)c1cccc(c1)C(=O)N(Cc1ncc(C3CC3)cn1)[C@H](CC(C)(C)C)CO2] | ++ | +++ | |
| 55 | 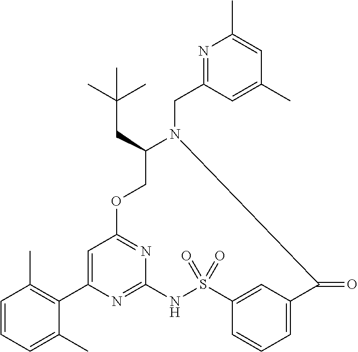 [SMILES: Cc1cc(C)nc(CN2C(=O)c3cccc(c3)S(=O)(=O)Nc3nc(cc(-c4c(C)cccc4C)n3)OC[C@H]2CC(C)(C)C)c1] | +++ | +++ | |
| 131 | 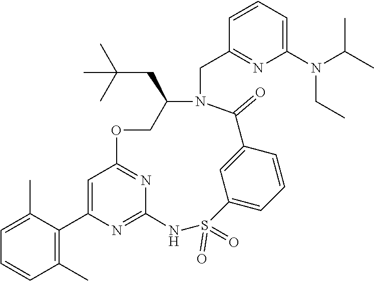 [SMILES: CCN(c1cccc(CN2C(=O)c3cccc(c3)S(=O)(=O)Nc3nc(cc(-c4c(C)cccc4C)n3)OC[C@H]2CC(C)(C)C)n1)C(C)C] | +++ | +++ | |
| 132 | 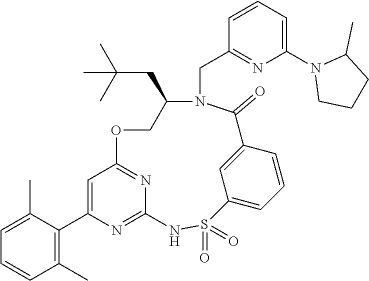 [SMILES: Cc1cccc(C)c1-c1cc2nc(n1)NS(=O)(=O)c1cccc(c1)C(=O)N(Cc1cccc(N3CCCC3C)n1)[C@H](CC(C)(C)C)CO2] | +++ | +++ | |
| 710 | 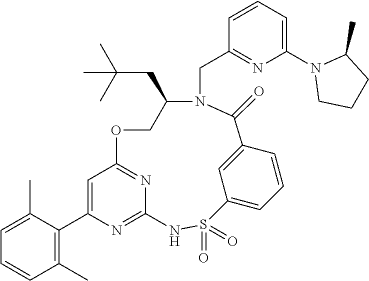 [SMILES: Cc1cccc(C)c1-c1cc2nc(n1)NS(=O)(=O)c1cccc(c1)C(=O)N(Cc1cccc(N3CCC[C@@H]3C)n1)[C@H](CC(C)(C)C)CO2] | +++ | +++ | |
| 709 | 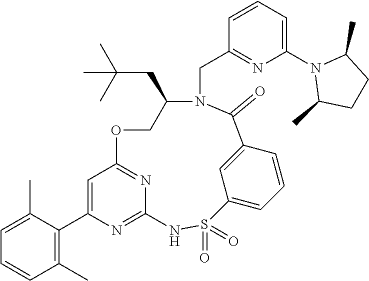 [SMILES: Cc1cccc(C)c1-c1cc2nc(n1)NS(=O)(=O)c1cccc(c1)C(=O)N(Cc1cccc(N3[C@H](C)CC[C@@H]3C)n1)[C@H](CC(C)(C)C)CO2] | +++ | ++ | |
| 638 | 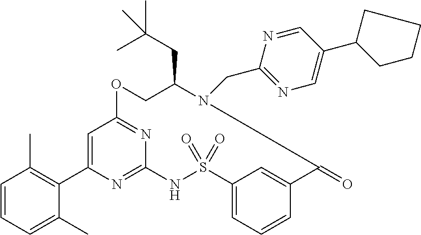 [SMILES: Cc1cccc(C)c1-c1cc2nc(n1)NS(=O)(=O)c1cccc(c1)C(=O)N(Cc1ncc(C3CCCC3)cn1)[C@H](CC(C)(C)C)CO2] | ++ | +++ | |
| 639 | 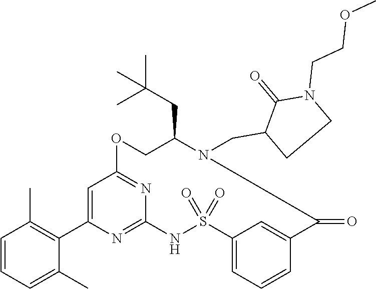 [SMILES: COCCN1CCC(CN2C(=O)c3cccc(c3)S(=O)(=O)Nc3nc(cc(-c4c(C)cccc4C)n3)OC[C@H]2CC(C)(C)C)C1=O] | +++ | +++ | |
| 640 | 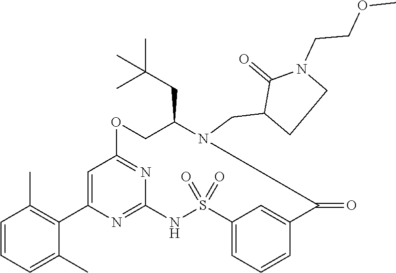 [SMILES: COCCN1CCC(CN2C(=O)c3cccc(c3)S(=O)(=O)Nc3nc(cc(-c4c(C)cccc4C)n3)OC[C@H]2CC(C)(C)C)C1=O] | +++ | +++ | |
| 641 | 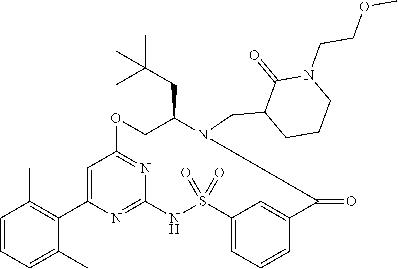 [SMILES: COCCN1CCCC(CN2C(=O)c3cccc(c3)S(=O)(=O)Nc3nc(cc(-c4c(C)cccc4C)n3)OC[C@H]2CC(C)(C)C)C1=O] | ++ | +++ | |
| 635 | 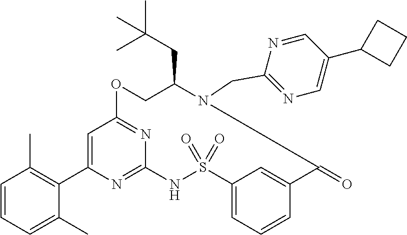 [SMILES: Cc1cccc(C)c1-c1cc2nc(n1)NS(=O)(=O)c1cccc(c1)C(=O)N(Cc1ncc(C3CCC3)cn1)[C@H](CC(C)(C)C)CO2] | + | +++ | |
| 636 | 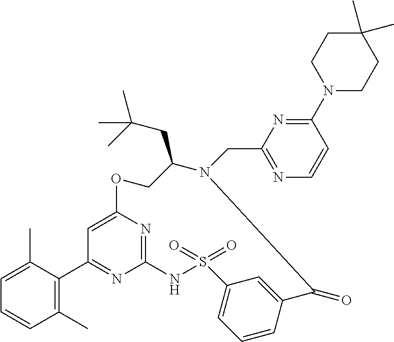 [SMILES: Cc1cccc(C)c1-c1cc2nc(n1)NS(=O)(=O)c1cccc(c1)C(=O)N(Cc1nccc(N3CCC(C)(C)CC3)n1)[C@H](CC(C)(C)C)CO2] | +++ | +++ | |
| 637 | 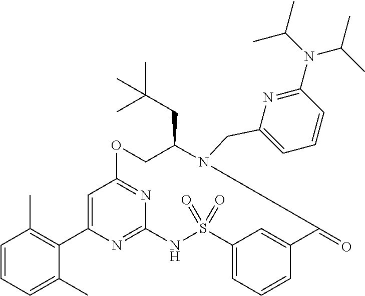 [SMILES: Cc1cccc(C)c1-c1cc2nc(n1)NS(=O)(=O)c1cccc(c1)C(=O)N(Cc1cccc(N(C(C)C)C(C)C)n1)[C@H](CC(C)(C)C)CO2] | + | +++ | |
| 123 | 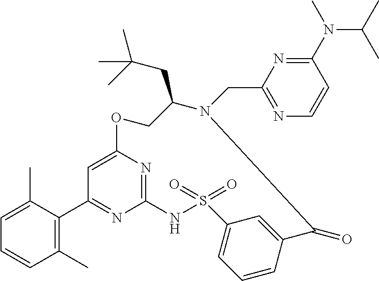 [SMILES: Cc1cccc(C)c1-c1cc2nc(n1)NS(=O)(=O)c1cccc(c1)C(=O)N(Cc1nccc(N(C)C(C)C)n1)[C@H](CC(C)(C)C)CO2] | +++ | +++ | |
| 634 | 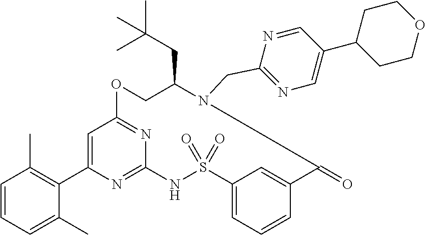 [SMILES: Cc1cccc(C)c1-c1cc2nc(n1)NS(=O)(=O)c1cccc(c1)C(=O)N(Cc1ncc(C3CCOCC3)cn1)[C@H](CC(C)(C)C)CO2] | +++ | +++ | |
| 133 | 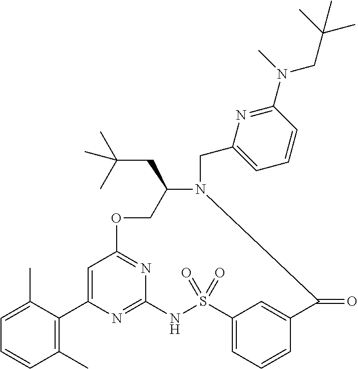 [SMILES: Cc1cccc(C)c1-c1cc2nc(n1)NS(=O)(=O)c1cccc(c1)C(=O)N(Cc1cccc(N(C)CC(C)(C)C)n1)[C@H](CC(C)(C)C)CO2] | +++ | +++ | |
| 633 | 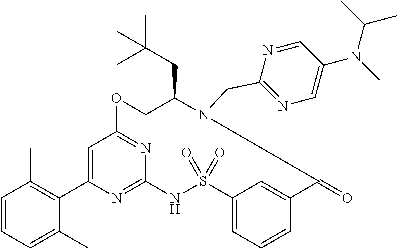 [SMILES: Cc1cccc(C)c1-c1cc2nc(n1)NS(=O)(=O)c1cccc(c1)C(=O)N(Cc1ncc(N(C)C(C)C)cn1)[C@H](CC(C)(C)C)CO2] | ++ | +++ | |
| 52 | 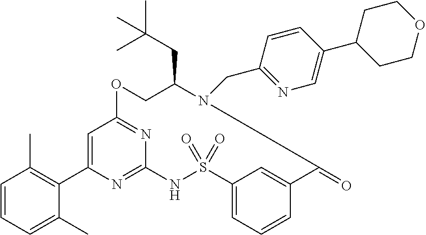 [SMILES: Cc1cccc(C)c1-c1cc2nc(n1)NS(=O)(=O)c1cccc(c1)C(=O)N(Cc1ccc(C3CCOCC3)cn1)[C@H](CC(C)(C)C)CO2] | +++ | +++ | |
| 120 | 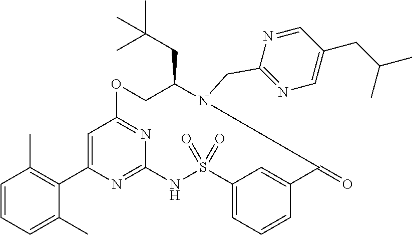 [SMILES: Cc1cccc(C)c1-c1cc2nc(n1)NS(=O)(=O)c1cccc(c1)C(=O)N(Cc1ncc(CC(C)C)cn1)[C@H](CC(C)(C)C)CO2] | +++ | +++ | |
| 632 | 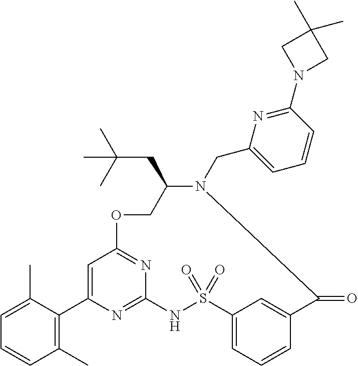 [SMILES: Cc1cccc(C)c1-c1cc2nc(n1)NS(=O)(=O)c1cccc(c1)C(=O)N(Cc1cccc(N3CC(C)(C)C3)n1)[C@H](CC(C)(C)C)CO2] | +++ | +++ | |
| 630 | 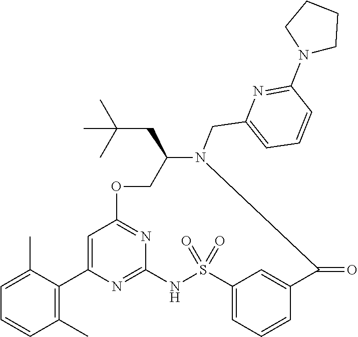 [SMILES: Cc1cccc(C)c1-c1cc2nc(n1)NS(=O)(=O)c1cccc(c1)C(=O)N(Cc1cccc(N3CCCC3)n1)[C@H](CC(C)(C)C)CO2] | +++ | +++ | |
| 631 | 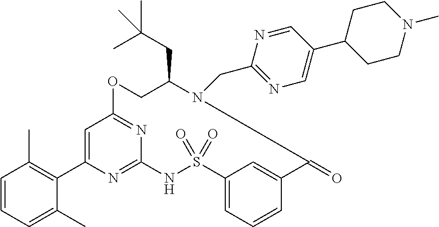 [SMILES: Cc1cccc(C)c1-c1cc2nc(n1)NS(=O)(=O)c1cccc(c1)C(=O)N(Cc1ncc(C3CCN(C)CC3)cn1)[C@H](CC(C)(C)C)CO2] | +++ | +++ | |
| 629 | 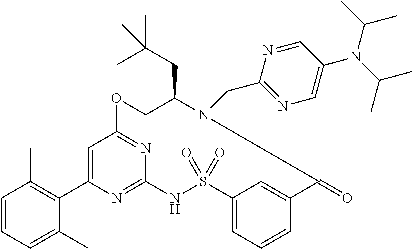 [SMILES: Cc1cccc(C)c1-c1cc2nc(n1)NS(=O)(=O)c1cccc(c1)C(=O)N(Cc1ncc(N(C(C)C)C(C)C)cn1)[C@H](CC(C)(C)C)CO2] | + | +++ | |
| 628 | 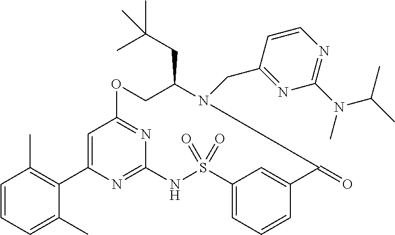 [SMILES: Cc1cccc(C)c1-c1cc2nc(n1)NS(=O)(=O)c1cccc(c1)C(=O)N(Cc1ccnc(N(C)C(C)C)n1)[C@H](CC(C)(C)C)CO2] | +++ | +++ | |
| 627 | 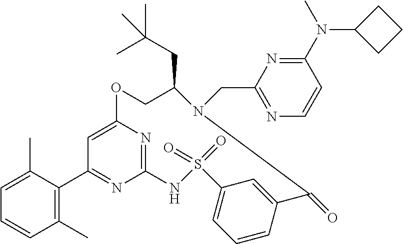 [SMILES: Cc1cccc(C)c1-c1cc2nc(n1)NS(=O)(=O)c1cccc(c1)C(=O)N(Cc1nccc(N(C)C3CCC3)n1)[C@H](CC(C)(C)C)CO2] | +++ | +++ | |
| 626 | 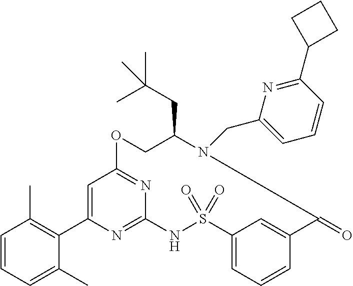 [SMILES: Cc1cccc(C)c1-c1cc2nc(n1)NS(=O)(=O)c1cccc(c1)C(=O)N(Cc1cccc(C3CCC3)n1)[C@H](CC(C)(C)C)CO2] | +++ | +++ | |
| 625 | 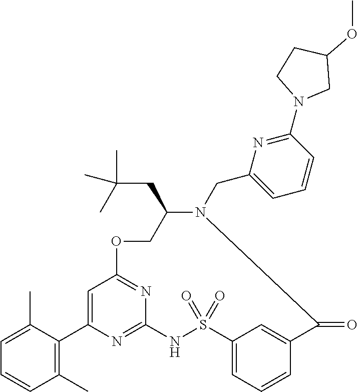 [SMILES: COC1CCN(c2cccc(CN3C(=O)c4cccc(c4)S(=O)(=O)Nc4nc(cc(-c5c(C)cccc5C)n4)OC[C@H]3CC(C)(C)C)n2)C1] | +++ | +++ | |
| 624 | 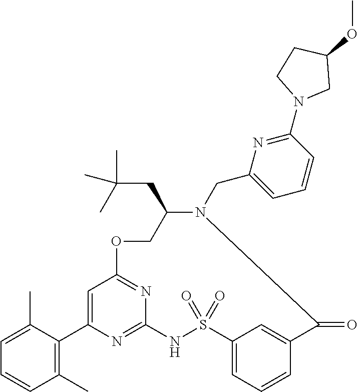 [SMILES: CO[C@@H]1CCN(c2cccc(CN3C(=O)c4cccc(c4)S(=O)(=O)Nc4nc(cc(-c5c(C)cccc5C)n4)OC[C@H]3CC(C)(C)C)n2)C1] | +++ | +++ | |
| 623 | 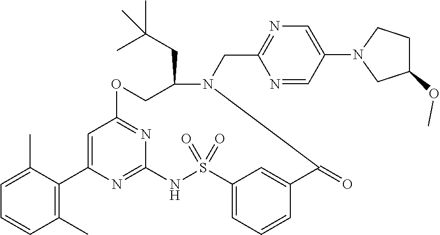 [SMILES: CO[C@@H]1CCN(c2cnc(CN3C(=O)c4cccc(c4)S(=O)(=O)Nc4nc(cc(-c5c(C)cccc5C)n4)OC[C@H]3CC(C)(C)C)nc2)C1] | +++ | +++ | |
| 622 | 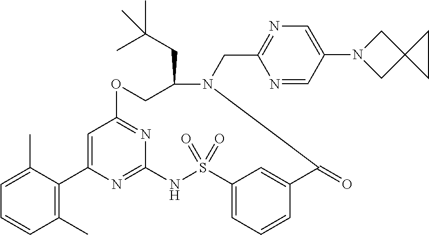 [SMILES: Cc1cccc(C)c1-c1cc2nc(n1)NS(=O)(=O)c1cccc(c1)C(=O)N(Cc1ncc(N3CC4(CC4)C3)cn1)[C@H](CC(C)(C)C)CO2] | ++ | +++ | |
| 621 | 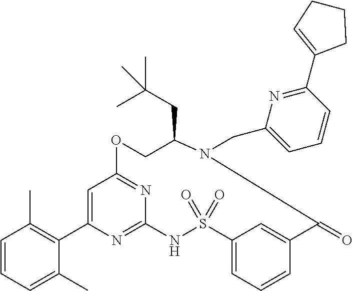 [SMILES: Cc1cccc(C)c1-c1cc2nc(n1)NS(=O)(=O)c1cccc(c1)C(=O)N(Cc1cccc(C3=CCCC3)n1)[C@H](CC(C)(C)C)CO2] | +++ | +++ | |
| 620 | 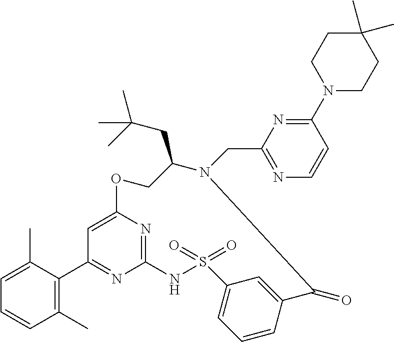 [SMILES: Cc1cccc(C)c1-c1cc2nc(n1)NS(=O)(=O)c1cccc(c1)C(=O)N(Cc1nccc(N3CCC(C)(C)CC3)n1)[C@H](CC(C)(C)C)CO2] | | + | +++ |
| 618 | 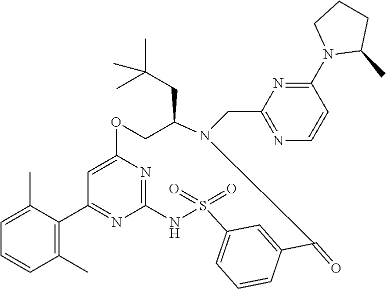 [SMILES: Cc1cccc(C)c1-c1cc2nc(n1)NS(=O)(=O)c1cccc(c1)C(=O)N(Cc1nccc(N3CCC[C@H]3C)n1)[C@H](CC(C)(C)C)CO2] | +++ | +++ | |
| 619 | 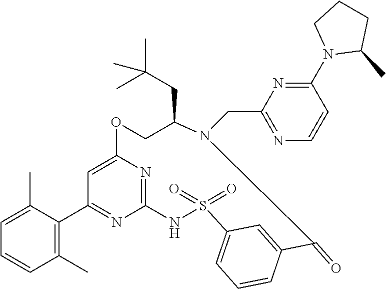 [SMILES: Cc1cccc(C)c1-c1cc2nc(n1)NS(=O)(=O)c1cccc(c1)C(=O)N(Cc1nccc(N3CCC[C@H]3C)n1)[C@H](CC(C)(C)C)CO2] | +++ | +++ | |
| 617 | 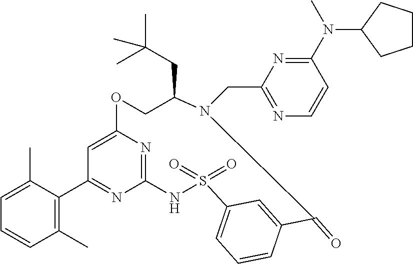 [SMILES: Cc1cccc(C)c1-c1cc2nc(n1)NS(=O)(=O)c1cccc(c1)C(=O)N(Cc1nccc(N(C)C3CCCC3)n1)[C@H](CC(C)(C)C)CO2] | ++ | +++ | |
| 615 | 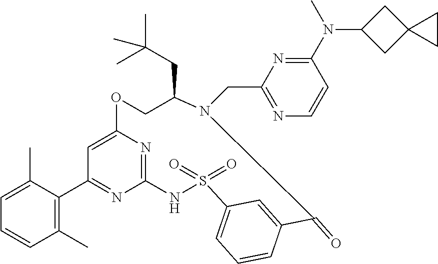 [SMILES: Cc1cccc(C)c1-c1cc2nc(n1)NS(=O)(=O)c1cccc(c1)C(=O)N(Cc1nccc(N(C)C3CC4(CC4)C3)n1)[C@H](CC(C)(C)C)CO2] | +++ | +++ | |
| 616 | 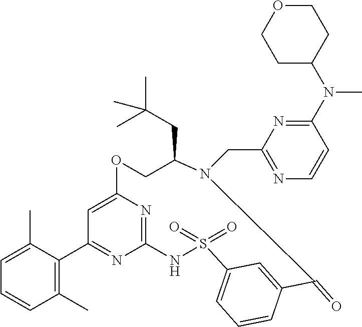 [SMILES: Cc1cccc(C)c1-c1cc2nc(n1)NS(=O)(=O)c1cccc(c1)C(=O)N(Cc1nccc(N(C)C3CCOCC3)n1)[C@H](CC(C)(C)C)CO2] | +++ | +++ | |
| 610 | 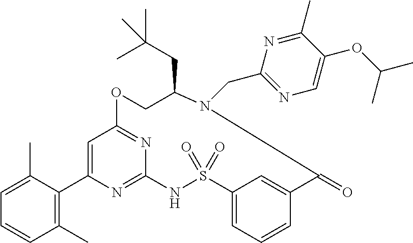 [SMILES: Cc1cccc(C)c1-c1cc2nc(n1)NS(=O)(=O)c1cccc(c1)C(=O)N(Cc1ncc(OC(C)C)c(C)n1)[C@H](CC(C)(C)C)CO2] | +++ | +++ | |
| 611 | 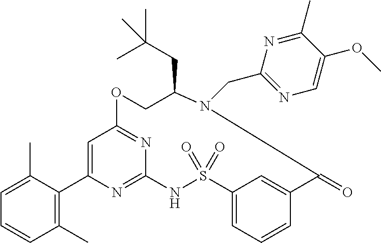 [SMILES: COc1cnc(CN2C(=O)c3cccc(c3)S(=O)(=O)Nc3nc(cc(-c4c(C)cccc4C)n3)OC[C@H]2CC(C)(C)C)nc1C] | +++ | +++ | |
| 614 | 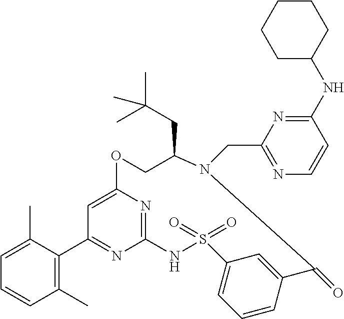 [SMILES: Cc1cccc(C)c1-c1cc2nc(n1)NS(=O)(=O)c1cccc(c1)C(=O)N(Cc1nccc(NC3CCCCC3)n1)[C@H](CC(C)(C)C)CO2] | +++ | +++ | |
| 609 | 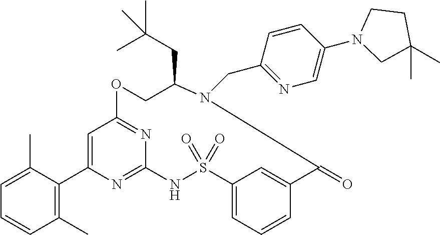 [SMILES: Cc1cccc(C)c1-c1cc2nc(n1)NS(=O)(=O)c1cccc(c1)C(=O)N(Cc1ccc(N3CCC(C)(C)C3)cn1)[C@H](CC(C)(C)C)CO2] | ++ | +++ | |
| 612 | 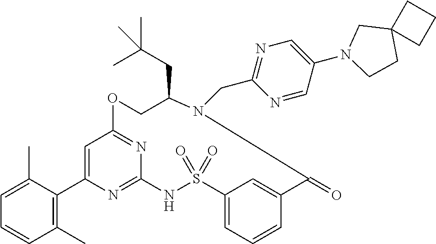 [SMILES: Cc1cccc(C)c1-c1cc2nc(n1)NS(=O)(=O)c1cccc(c1)C(=O)N(Cc1ncc(N3CCC4(CCC4)C3)cn1)[C@H](CC(C)(C)C)CO2] | +++ | +++ | |
| 613 | 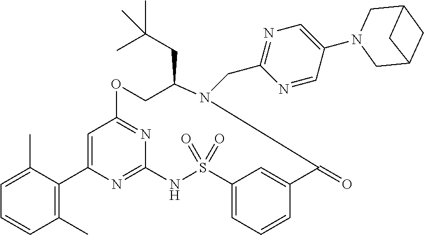 [SMILES: Cc1cccc(C)c1-c1cc2nc(n1)NS(=O)(=O)c1cccc(c1)C(=O)N(Cc1ncc(N3CC4CC(C4)C3)cn1)[C@H](CC(C)(C)C)CO2] | +++ | +++ | |
| 608 | 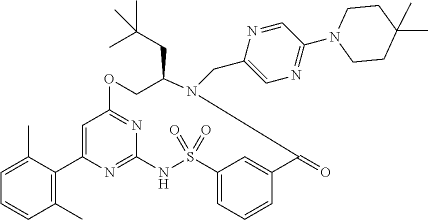 [SMILES: Cc1cccc(C)c1-c1cc2nc(n1)NS(=O)(=O)c1cccc(c1)C(=O)N(Cc1cnc(N3CCC(C)(C)CC3)cn1)[C@H](CC(C)(C)C)CO2] | +++ | +++ | |
| 607 | 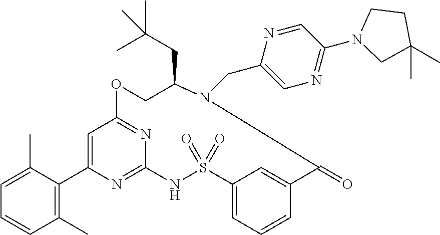 [SMILES: Cc1cccc(C)c1-c1cc2nc(n1)NS(=O)(=O)c1cccc(c1)C(=O)N(Cc1cnc(N3CCC(C)(C)C3)cn1)[C@H](CC(C)(C)C)CO2] | +++ | +++ | |
| 331 | 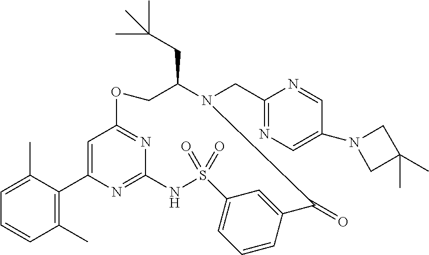 [SMILES: Cc1cccc(C)c1-c1cc2nc(n1)NS(=O)(=O)c1cccc(c1)C(=O)N(Cc1ncc(N3CC(C)(C)C3)cn1)[C@H](CC(C)(C)C)CO2] | +++ | +++ | |
| 332 | 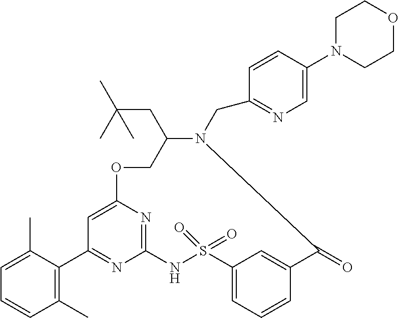 [SMILES: Cc1cccc(C)c1-c1cc2nc(n1)NS(=O)(=O)c1cccc(c1)C(=O)N(Cc1ccc(N3CCOCC3)cn1)C(CC(C)(C)C)CO2] | | | + |
| 606 | 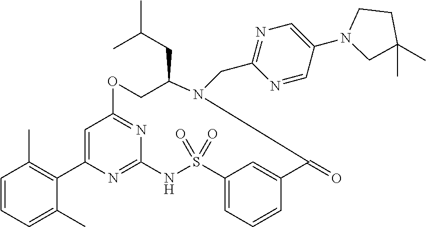 [SMILES: Cc1cccc(C)c1-c1cc2nc(n1)NS(=O)(=O)c1cccc(c1)C(=O)N(Cc1ncc(N3CCC(C)(C)C3)cn1)[C@H](CC(C)C)CO2] | +++ | +++ | |
| 605 | 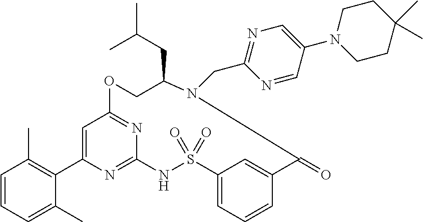 [SMILES: Cc1cccc(C)c1-c1cc2nc(n1)NS(=O)(=O)c1cccc(c1)C(=O)N(Cc1ncc(N3CCC(C)(C)CC3)cn1)[C@H](CC(C)C)CO2] | + | +++ | |
| 599 | 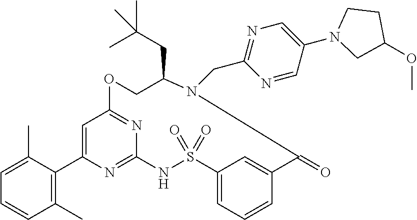 [SMILES: COC1CCN(c2cnc(CN3C(=O)c4cccc(c4)S(=O)(=O)Nc4nc(cc(-c5c(C)cccc5C)n4)OC[C@H]3CC(C)(C)C)nc2)C1] | ++ | +++ | |
| 598 | 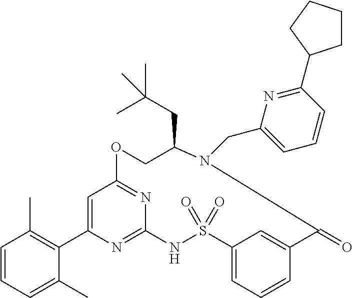 [SMILES: Cc1cccc(C)c1-c1cc2nc(n1)NS(=O)(=O)c1cccc(c1)C(=O)N(Cc1cccc(C3CCCC3)n1)[C@H](CC(C)(C)C)CO2] | +++ | +++ | +++ |
| 600 | 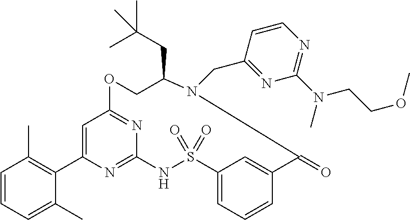 [SMILES: COCCN(C)c1nccc(CN2C(=O)c3cccc(c3)S(=O)(=O)Nc3nc(cc(-c4c(C)cccc4C)n3)OC[C@H]2CC(C)(C)C)n1] | +++ | +++ | |
| 601 | 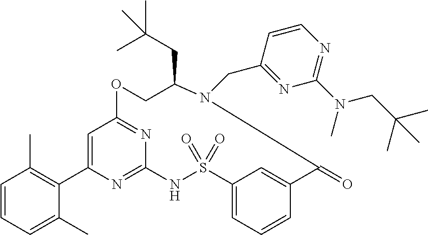 [SMILES: Cc1cccc(C)c1-c1cc2nc(n1)NS(=O)(=O)c1cccc(c1)C(=O)N(Cc1ccnc(N(C)CC(C)(C)C)n1)[C@H](CC(C)(C)C)CO2] | +++ | +++ | |
| 602 | 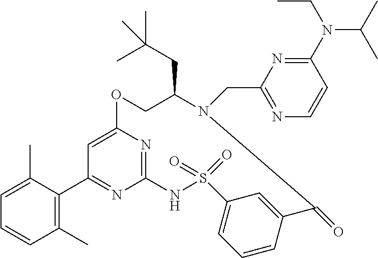 [SMILES: CCN(c1ccnc(CN2C(=O)c3cccc(c3)S(=O)(=O)Nc3nc(cc(-c4c(C)cccc4C)n3)OC[C@H]2CC(C)(C)C)n1)C(C)C] | +++ | +++ | |
| 603 | 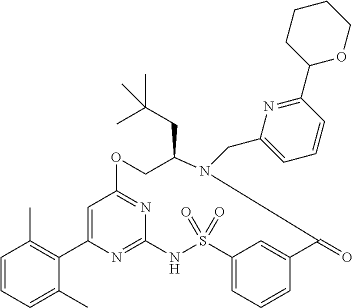 [SMILES: Cc1cccc(C)c1-c1cc2nc(n1)NS(=O)(=O)c1cccc(c1)C(=O)N(Cc1cccc(C3CCCCO3)n1)[C@H](CC(C)(C)C)CO2] | +++ | +++ | |

9. Biological Activity Data

Table 12 represents CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” % Activity: +++ is >600%; ++ is 30-60%; + is <30%).

TABLE 12

| | | | HBE | HBE |
|---|---|---|---|---|
| | | HBE | Max | Activity |
| Cmpd | | EC₅₀ | Activity | at 10 μM |
| Number | Structure | (μM) | (%) | (%) |
| 604 | 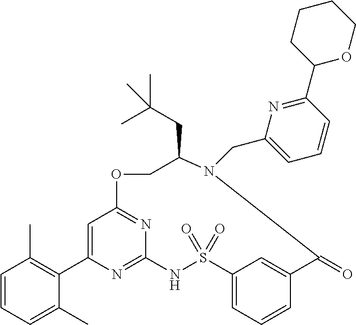 [SMILES: Cc1cccc(C)c1-c1cc2nc(n1)NS(=O)(=O)c1cccc(c1)C(=O)N(Cc1cccc(C3CCCCO3)n1)[C@H](CC(C)(C)C)CO2] | +++ | +++ | |
| 597 | 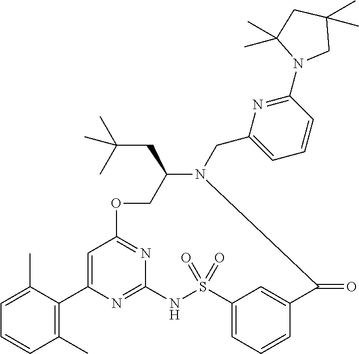 [SMILES: Cc1cccc(C)c1-c1cc2nc(n1)NS(=O)(=O)c1cccc(c1)C(=O)N(Cc1cccc(N3CC(C)(C)CC3(C)C)n1)[C@H](CC(C)(C)C)CO2] | +++ | ++ | |
| 596 | 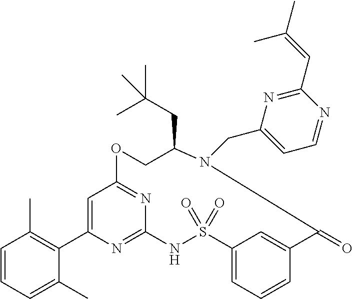 [SMILES: CC(C)=Cc1nccc(CN2C(=O)c3cccc(c3)S(=O)(=O)Nc3nc(cc(-c4c(C)cccc4C)n3)OC[C@H]2CC(C)(C)C)n1] | +++ | +++ | |
| 595 | 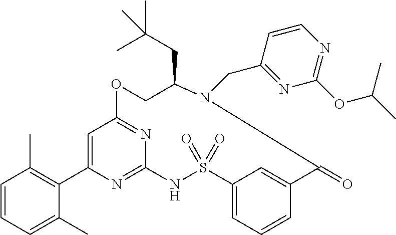 [SMILES: Cc1cccc(C)c1-c1cc2nc(n1)NS(=O)(=O)c1cccc(c1)C(=O)N(Cc1ccnc(OC(C)C)n1)[C@H](CC(C)(C)C)CO2] | +++ | +++ | |
| 594 | 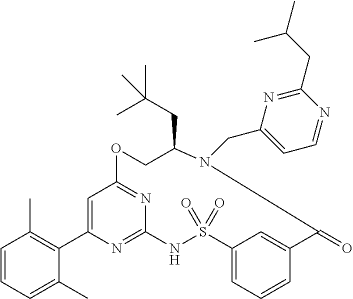 [SMILES: Cc1cccc(C)c1-c1cc2nc(n1)NS(=O)(=O)c1cccc(c1)C(=O)N(Cc1ccnc(CC(C)C)n1)[C@H](CC(C)(C)C)CO2] | +++ | +++ | |
| 593 | 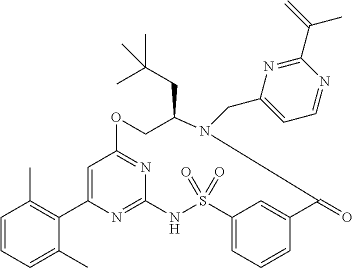 [SMILES: C=C(C)c1nccc(CN2C(=O)c3cccc(c3)S(=O)(=O)Nc3nc(cc(-c4c(C)cccc4C)n3)OC[C@H]2CC(C)(C)C)n1] | +++ | +++ | |
| 592 | 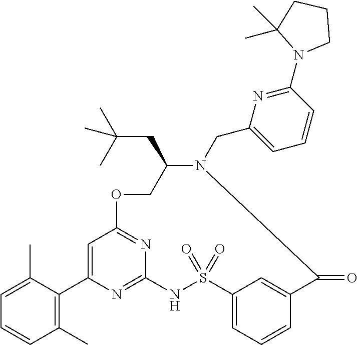 [SMILES: Cc1cccc(C)c1-c1cc2nc(n1)NS(=O)(=O)c1cccc(c1)C(=O)N(Cc1cccc(N3CCCC3(C)C)n1)[C@H](CC(C)(C)C)CO2] | ++ | +++ | |
| 591 | 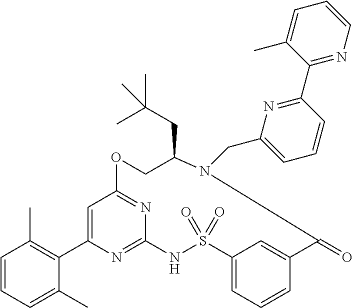 [SMILES: Cc1cccnc1-c1cccc(CN2C(=O)c3cccc(c3)S(=O)(=O)Nc3nc(cc(-c4c(C)cccc4C)n3)OC[C@H]2CC(C)(C)C)n1] | | | +++ |
| 590 | 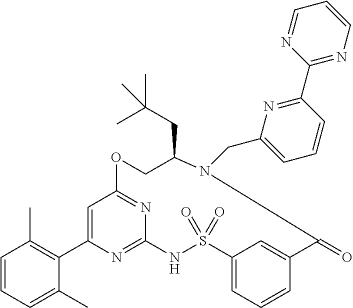 [SMILES: Cc1cccc(C)c1-c1cc2nc(n1)NS(=O)(=O)c1cccc(c1)C(=O)N(Cc1cccc(-c3ncccn3)n1)[C@H](CC(C)(C)C)CO2] | +++ | +++ | |
| 589 | 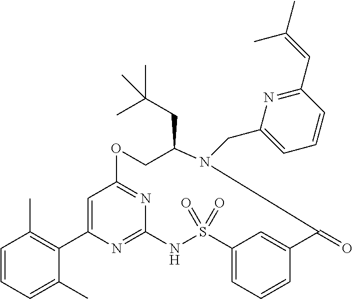 [SMILES: CC(C)=Cc1cccc(CN2C(=O)c3cccc(c3)S(=O)(=O)Nc3nc(cc(-c4c(C)cccc4C)n3)OC[C@H]2CC(C)(C)C)n1] | +++ | +++ | |
| 588 | 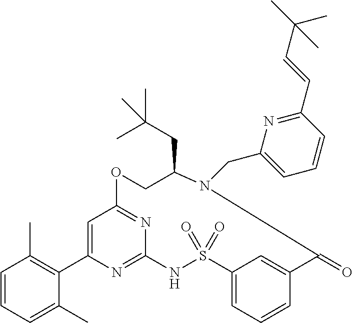 [SMILES: Cc1cccc(C)c1-c1cc2nc(n1)NS(=O)(=O)c1cccc(c1)C(=O)N(Cc1cccc(/C=C/C(C)(C)C)n1)[C@H](CC(C)(C)C)CO2] | +++ | +++ | |
| 587 | 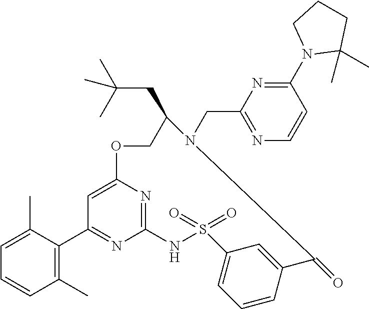 [SMILES: Cc1cccc(C)c1-c1cc2nc(n1)NS(=O)(=O)c1cccc(c1)C(=O)N(Cc1nccc(N3CCCC3(C)C)n1)[C@H](CC(C)(C)C)CO2] | +++ | +++ | |
| 586 | 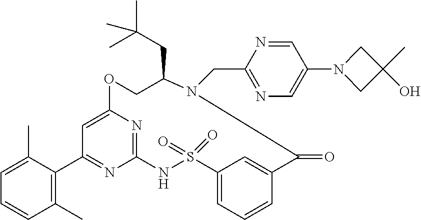 [SMILES: Cc1cccc(C)c1-c1cc2nc(n1)NS(=O)(=O)c1cccc(c1)C(=O)N(Cc1ncc(N3CC(C)(O)C3)cn1)[C@H](CC(C)(C)C)CO2] | +++ | +++ | |
| 585 | 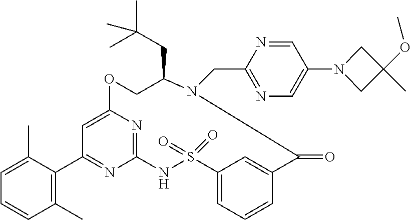 [SMILES: COC1(C)CN(c2cnc(CN3C(=O)c4cccc(c4)S(=O)(=O)Nc4nc(cc(-c5c(C)cccc5C)n4)OC[C@H]3CC(C)(C)C)nc2)C1] | +++ | +++ | |
| 155 | 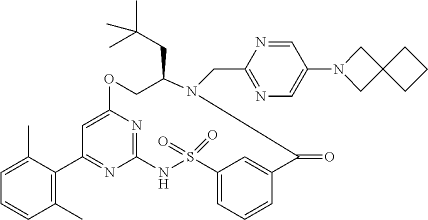 [SMILES: Cc1cccc(C)c1-c1cc2nc(n1)NS(=O)(=O)c1cccc(c1)C(=O)N(Cc1ncc(N3CC4(CCC4)C3)cn1)[C@H](CC(C)(C)C)CO2] | +++ | +++ | |
| 584 | 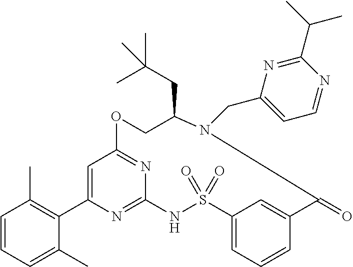 [SMILES: Cc1cccc(C)c1-c1cc2nc(n1)NS(=O)(=O)c1cccc(c1)C(=O)N(Cc1ccnc(C(C)C)n1)[C@H](CC(C)(C)C)CO2] | +++ | +++ | |
| 583 | 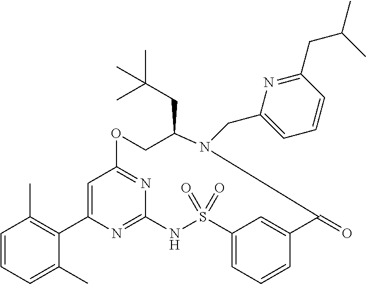 [SMILES: Cc1cccc(C)c1-c1cc2nc(n1)NS(=O)(=O)c1cccc(c1)C(=O)N(Cc1cccc(CC(C)C)n1)[C@H](CC(C)(C)C)CO2] | +++ | +++ | |
| 582 | 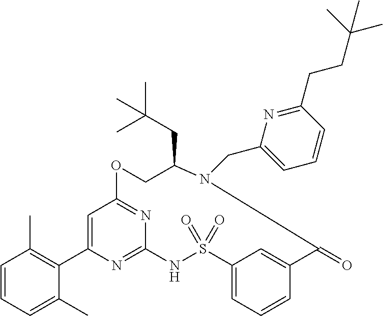 [SMILES: Cc1cccc(C)c1-c1cc2nc(n1)NS(=O)(=O)c1cccc(c1)C(=O)N(Cc1cccc(CCC(C)(C)C)n1)[C@H](CC(C)(C)C)CO2] | +++ | +++ | |
| 576 | 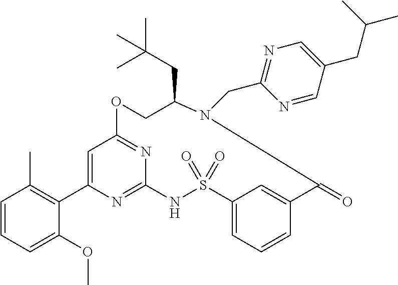 [SMILES: COc1cccc(C)c1-c1cc2nc(n1)NS(=O)(=O)c1cccc(c1)C(=O)N(Cc1ncc(CC(C)C)cn1)[C@H](CC(C)(C)C)CO2] | | | +++ |
| 578 | 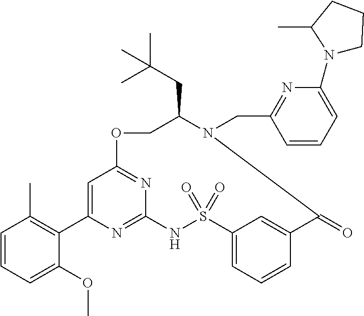 [SMILES: COc1cccc(C)c1-c1cc2nc(n1)NS(=O)(=O)c1cccc(c1)C(=O)N(Cc1cccc(N3CCCC3C)n1)[C@H](CC(C)(C)C)CO2] | | | + |
| 579 | 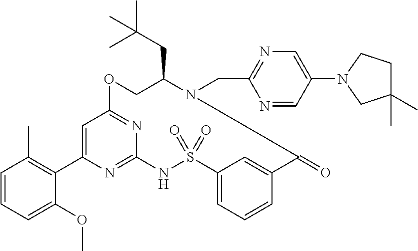 [SMILES: COc1cccc(C)c1-c1cc2nc(n1)NS(=O)(=O)c1cccc(c1)C(=O)N(Cc1ncc(N3CCC(C)(C)C3)cn1)[C@H](CC(C)(C)C)CO2] | +++ | +++ | |
| 580 | 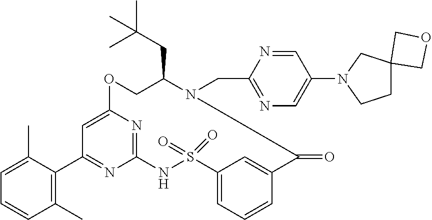 [SMILES: Cc1cccc(C)c1-c1cc2nc(n1)NS(=O)(=O)c1cccc(c1)C(=O)N(Cc1ncc(N3CCC4(COC4)C3)cn1)[C@H](CC(C)(C)C)CO2] | +++ | +++ | |
| 581 | 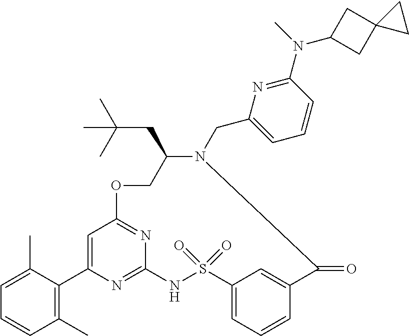 [SMILES: Cc1cccc(C)c1-c1cc2nc(n1)NS(=O)(=O)c1cccc(c1)C(=O)N(Cc1cccc(N(C)C3CC4(CC4)C3)n1)[C@H](CC(C)(C)C)CO2] | +++ | +++ | |
| 463 | 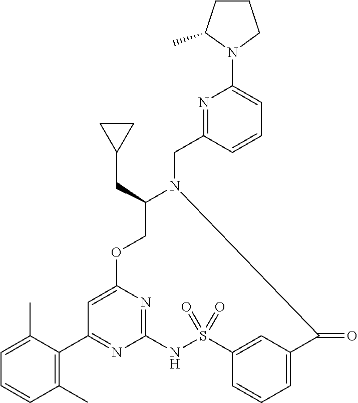 [SMILES: Cc1cccc(C)c1-c1cc2nc(n1)NS(=O)(=O)c1cccc(c1)C(=O)N(Cc1cccc(N3CCC[C@H]3C)n1)[C@H](CC1CC1)CO2] | +++ | +++ | |
| 462 | 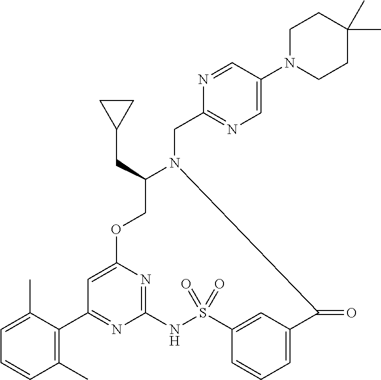 [SMILES: Cc1cccc(C)c1-c1cc2nc(n1)NS(=O)(=O)c1cccc(c1)C(=O)N(Cc1ncc(N3CCC(C)(C)CC3)cn1)[C@H](CC1CC1)CO2] | +++ | +++ | |
| 461 | 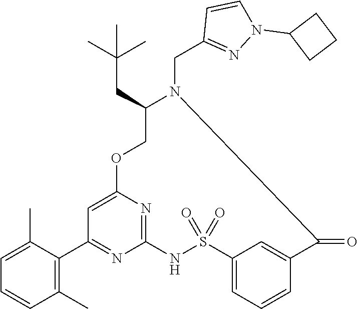 [SMILES: Cc1cccc(C)c1-c1cc2nc(n1)NS(=O)(=O)c1cccc(c1)C(=O)N(Cc1ccn(C3CCC3)n1)[C@H](CC(C)(C)C)CO2] | +++ | +++ | |
| 460 | 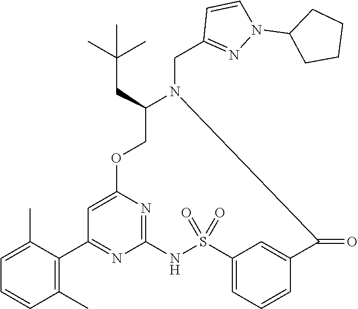 [SMILES: Cc1cccc(C)c1-c1cc2nc(n1)NS(=O)(=O)c1cccc(c1)C(=O)N(Cc1ccn(C3CCCC3)n1)[C@H](CC(C)(C)C)CO2] | +++ | +++ | |
| 285 | 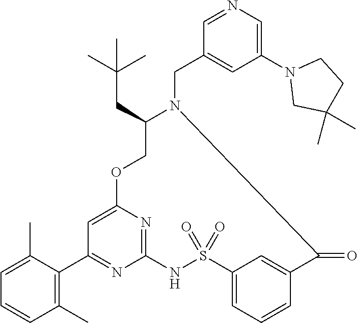 [SMILES: Cc1cccc(C)c1-c1cc2nc(n1)NS(=O)(=O)c1cccc(c1)C(=O)N(Cc1cncc(N3CCC(C)(C)C3)c1)[C@H](CC(C)(C)C)CO2] | +++ | +++ | |
| 330 | 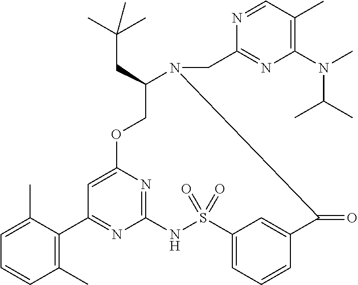 [SMILES: Cc1cnc(CN2C(=O)c3cccc(c3)S(=O)(=O)Nc3nc(cc(-c4c(C)cccc4C)n3)OC[C@H]2CC(C)(C)C)nc1N(C)C(C)C] | | | +++ |
| 329 | 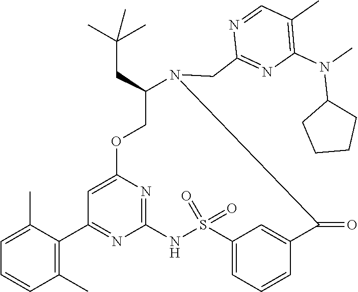 [SMILES: Cc1cnc(CN2C(=O)c3cccc(c3)S(=O)(=O)Nc3nc(cc(-c4c(C)cccc4C)n3)OC[C@H]2CC(C)(C)C)nc1N(C)C1CCCC1] | | | +++ |
| 328 | 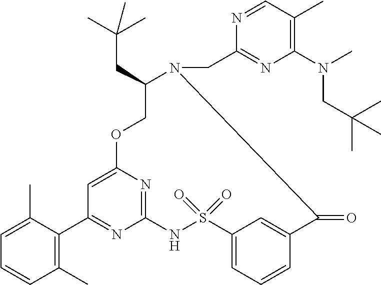 [SMILES: Cc1cnc(CN2C(=O)c3cccc(c3)S(=O)(=O)Nc3nc(cc(-c4c(C)cccc4C)n3)OC[C@H]2CC(C)(C)C)nc1N(C)CC(C)(C)C] | +++ | +++ | |
| 527 | 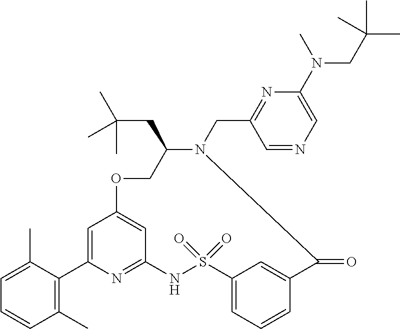 [SMILES: Cc1cccc(C)c1-c1cc2cc(n1)NS(=O)(=O)c1cccc(c1)C(=O)N(Cc1cncc(N(C)CC(C)(C)C)n1)[C@H](CC(C)(C)C)CO2] | +++ | +++ | |
| 459 | 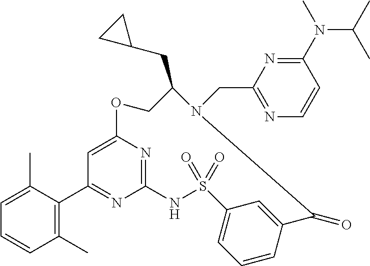 [SMILES: Cc1cccc(C)c1-c1cc2nc(n1)NS(=O)(=O)c1cccc(c1)C(=O)N(Cc1nccc(N(C)C(C)C)n1)[C@H](CC1CC1)CO2] | +++ | +++ | |
| 458 | 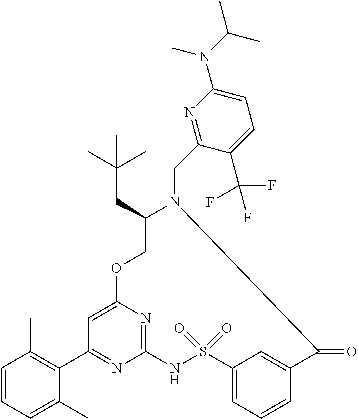 [SMILES: Cc1cccc(C)c1-c1cc2nc(n1)NS(=O)(=O)c1cccc(c1)C(=O)N(Cc1nc(N(C)C(C)C)ccc1C(F)(F)F)[C@H](CC(C)(C)C)CO2] | | | +++ |
| 457 | 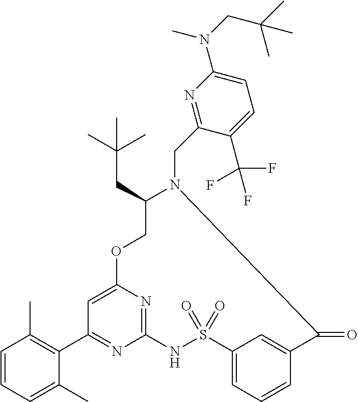 [SMILES: Cc1cccc(C)c1-c1cc2nc(n1)NS(=O)(=O)c1cccc(c1)C(=O)N(Cc1nc(N(C)CC(C)(C)C)ccc1C(F)(F)F)[C@H](CC(C)(C)C)CO2] | +++ | +++ | |
| 286 | 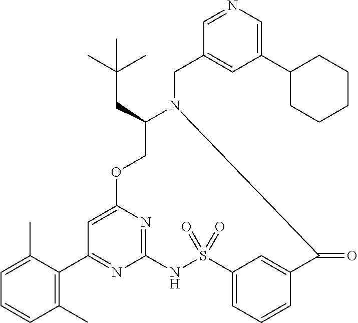 [SMILES: Cc1cccc(C)c1-c1cc2nc(n1)NS(=O)(=O)c1cccc(c1)C(=O)N(Cc1cncc(C3CCCCC3)c1)[C@H](CC(C)(C)C)CO2] | +++ | +++ | |
| 238 | 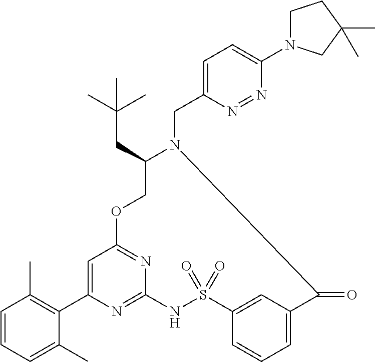 [SMILES: Cc1cccc(C)c1-c1cc2nc(n1)NS(=O)(=O)c1cccc(c1)C(=O)N(Cc1ccc(N3CCC(C)(C)C3)nn1)[C@H](CC(C)(C)C)CO2] | +++ | ++ | |
| 883 | 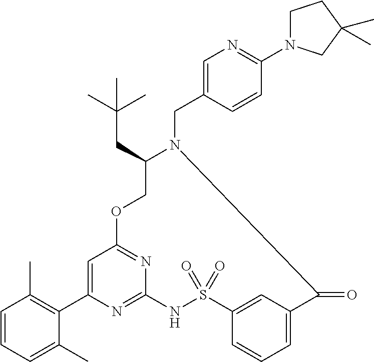 [SMILES: Cc1cccc(C)c1-c1cc2nc(n1)NS(=O)(=O)c1cccc(c1)C(=O)N(Cc1ccc(N3CCC(C)(C)C3)nc1)[C@H](CC(C)(C)C)CO2] | +++ | +++ | |
| 882 | 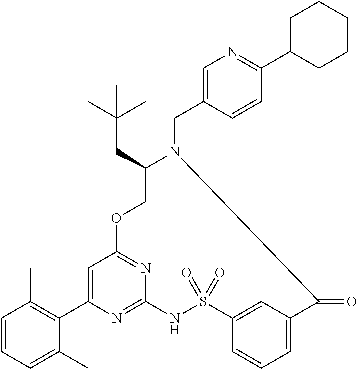 [SMILES: Cc1cccc(C)c1-c1cc2nc(n1)NS(=O)(=O)c1cccc(c1)C(=O)N(Cc1ccc(C3CCCCC3)nc1)[C@H](CC(C)(C)C)CO2] | | | +++ |
| 272 | 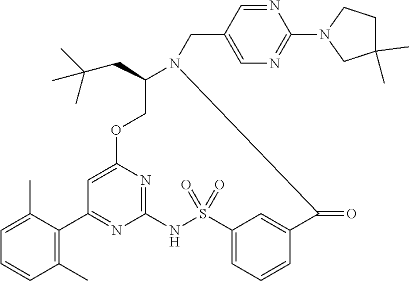 [SMILES: Cc1cccc(C)c1-c1cc2nc(n1)NS(=O)(=O)c1cccc(c1)C(=O)N(Cc1cnc(N3CCC(C)(C)C3)nc1)[C@H](CC(C)(C)C)CO2] | +++ | +++ | |
| 271 | 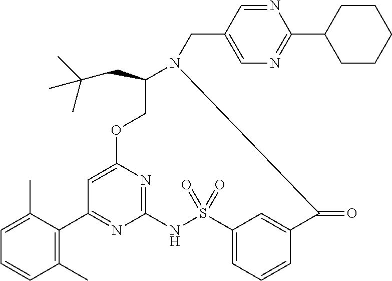 [SMILES: Cc1cccc(C)c1-c1cc2nc(n1)NS(=O)(=O)c1cccc(c1)C(=O)N(Cc1cnc(C3CCCCC3)nc1)[C@H](CC(C)(C)C)CO2] | +++ | +++ | |
| 270 | 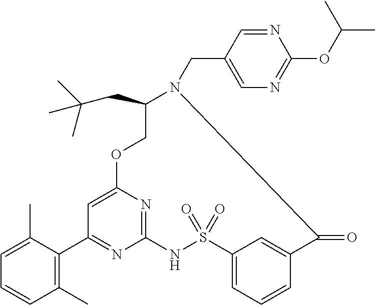 [SMILES: Cc1cccc(C)c1-c1cc2nc(n1)NS(=O)(=O)c1cccc(c1)C(=O)N(Cc1cnc(OC(C)C)nc1)[C@H](CC(C)(C)C)CO2] | +++ | +++ | |
| 1009 | 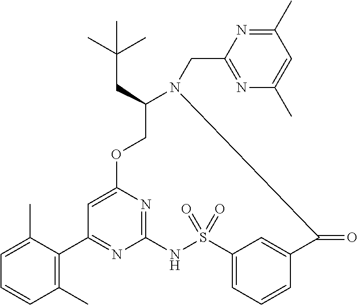 [SMILES: Cc1cc(C)nc(CN2C(=O)c3cccc(c3)S(=O)(=O)Nc3nc(cc(-c4c(C)cccc4C)n3)OC[C@H]2CC(C)(C)C)n1] | +++ | +++ | |
| 250 | 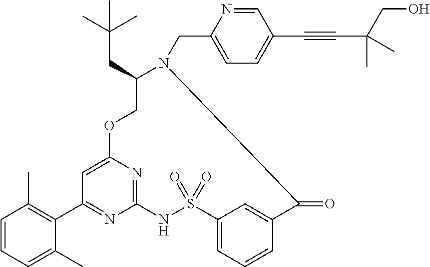 [SMILES: Cc1cccc(C)c1-c1cc2nc(n1)NS(=O)(=O)c1cccc(c1)C(=O)N(Cc1ccc(C#CC(C)(C)CO)cn1)[C@H](CC(C)(C)C)CO2] | +++ | +++ | |
| 1065 | 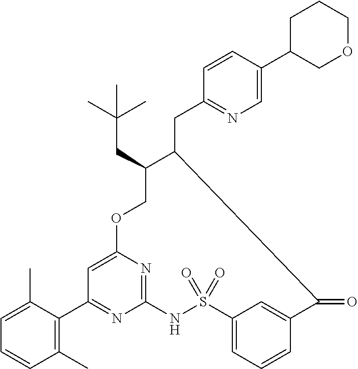 [SMILES: Cc1cccc(C)c1-c1cc2nc(n1)NS(=O)(=O)c1cccc(c1)C(=O)C(Cc1ccc(C3CCCOC3)cn1)[C@H](CC(C)(C)C)CO2] | +++ | +++ | |
| 261 | 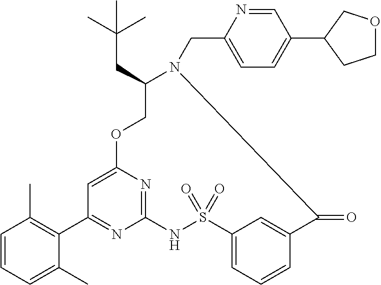 [SMILES: Cc1cccc(C)c1-c1cc2nc(n1)NS(=O)(=O)c1cccc(c1)C(=O)N(Cc1ccc(C3CCOC3)cn1)[C@H](CC(C)(C)C)CO2] | +++ | +++ | |
| 456 | 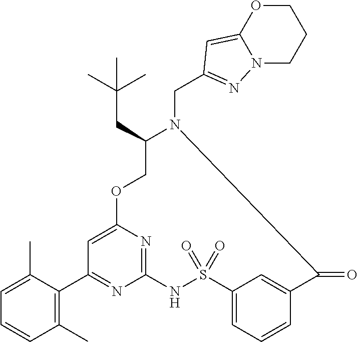 [SMILES: Cc1cccc(C)c1-c1cc2nc(n1)NS(=O)(=O)c1cccc(c1)C(=O)N(Cc1cc3n(n1)CCCO3)[C@H](CC(C)(C)C)CO2] | | | +++ |
| 455 | 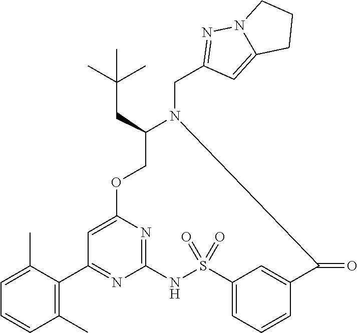 [SMILES: Cc1cccc(C)c1-c1cc2nc(n1)NS(=O)(=O)c1cccc(c1)C(=O)N(Cc1cc3n(n1)CCC3)[C@H](CC(C)(C)C)CO2] | | | +++ |
| 454 | 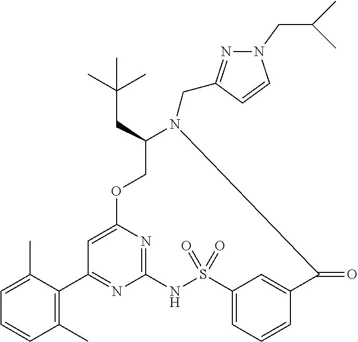 [SMILES: Cc1cccc(C)c1-c1cc2nc(n1)NS(=O)(=O)c1cccc(c1)C(=O)N(Cc1ccn(CC(C)C)n1)[C@H](CC(C)(C)C)CO2] | +++ | +++ | |
| 284 | 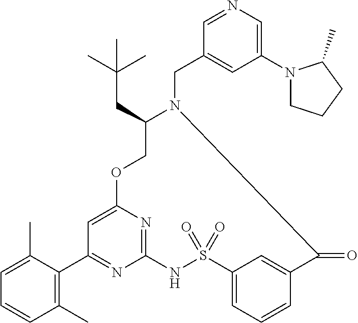 [SMILES: Cc1cccc(C)c1-c1cc2nc(n1)NS(=O)(=O)c1cccc(c1)C(=O)N(Cc1cncc(N3CCC[C@H]3C)c1)[C@H](CC(C)(C)C)CO2] | | | +++ |
| 283 | 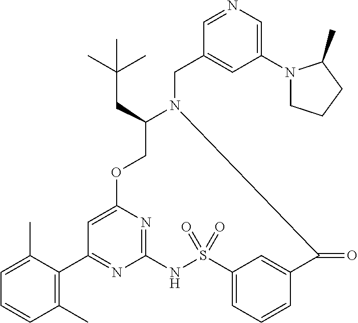 [SMILES: Cc1cccc(C)c1-c1cc2nc(n1)NS(=O)(=O)c1cccc(c1)C(=O)N(Cc1cncc(N3CCC[C@@H]3C)c1)[C@H](CC(C)(C)C)CO2] | | | +++ |
| 233 | 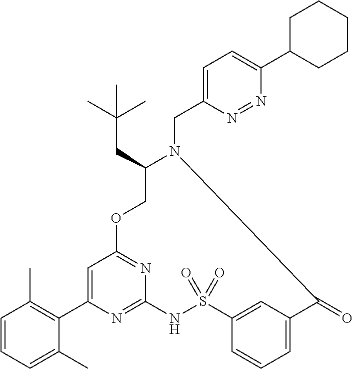 [SMILES: Cc1cccc(C)c1-c1cc2nc(n1)NS(=O)(=O)c1cccc(c1)C(=O)N(Cc1ccc(C3CCCCC3)nn1)[C@H](CC(C)(C)C)CO2] | | | +++ |
| 282 | 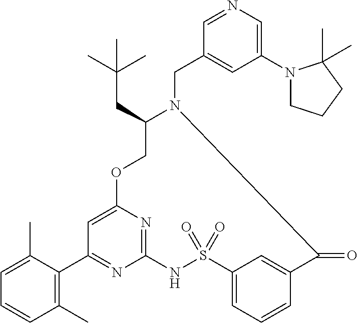 [SMILES: Cc1cccc(C)c1-c1cc2nc(n1)NS(=O)(=O)c1cccc(c1)C(=O)N(Cc1cncc(N3CCCC3(C)C)c1)[C@H](CC(C)(C)C)CO2] | | | +++ |
| 237 | 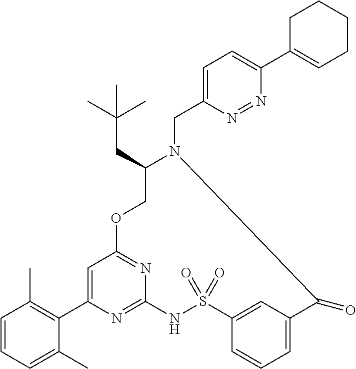 [SMILES: Cc1cccc(C)c1-c1cc2nc(n1)NS(=O)(=O)c1cccc(c1)C(=O)N(Cc1ccc(C3=CCCCC3)nn1)[C@H](CC(C)(C)C)CO2] | | | +++ |
| 281 | 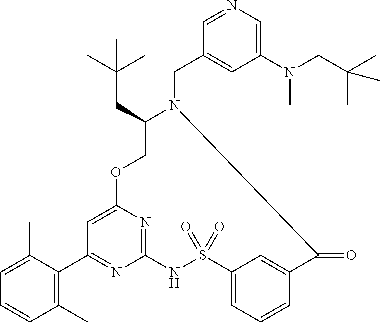 [SMILES: Cc1cccc(C)c1-c1cc2nc(n1)NS(=O)(=O)c1cccc(c1)C(=O)N(Cc1cncc(N(C)CC(C)(C)C)c1)[C@H](CC(C)(C)C)CO2] | | | +++ |
| 881 | 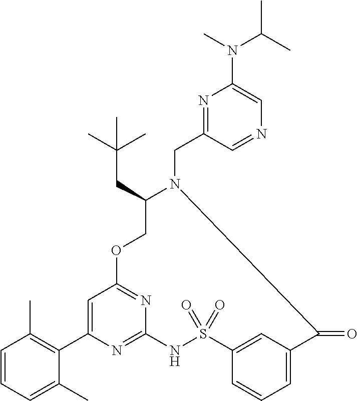 [SMILES: Cc1cccc(C)c1-c1cc2nc(n1)NS(=O)(=O)c1cccc(c1)C(=O)N(Cc1cncc(N(C)C(C)C)n1)[C@H](CC(C)(C)C)CO2] | +++ | +++ | |
| 880 | 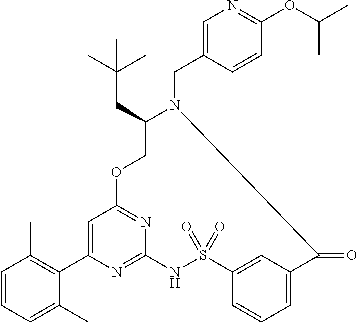 [SMILES: Cc1cccc(C)c1-c1cc2nc(n1)NS(=O)(=O)c1cccc(c1)C(=O)N(Cc1ccc(OC(C)C)nc1)[C@H](CC(C)(C)C)CO2] | | | +++ |
| 705 | 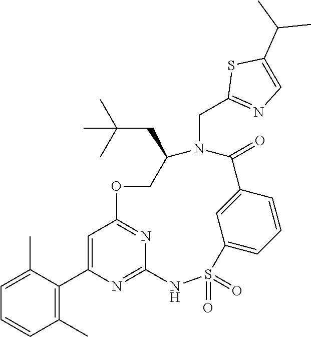 [SMILES: Cc1cccc(C)c1-c1cc2nc(n1)NS(=O)(=O)c1cccc(c1)C(=O)N(Cc1ncc(C(C)C)s1)[C@H](CC(C)(C)C)CO2] | +++ | +++ | |
| 704 | 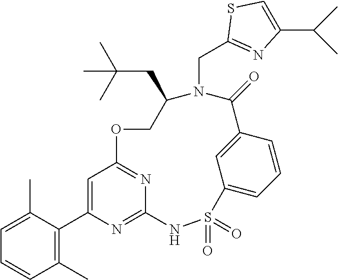 [SMILES: Cc1cccc(C)c1-c1cc2nc(n1)NS(=O)(=O)c1cccc(c1)C(=O)N(Cc1nc(C(C)C)cs1)[C@H](CC(C)(C)C)CO2] | +++ | +++ | |
| 703 | 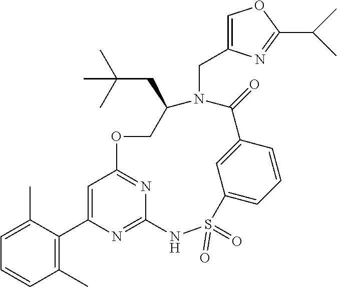 [SMILES: Cc1cccc(C)c1-c1cc2nc(n1)NS(=O)(=O)c1cccc(c1)C(=O)N(Cc1coc(C(C)C)n1)[C@H](CC(C)(C)C)CO2] | +++ | +++ | |
| 702 | 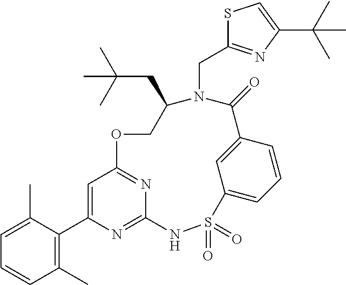 [SMILES: Cc1cccc(C)c1-c1cc2nc(n1)NS(=O)(=O)c1cccc(c1)C(=O)N(Cc1nc(C(C)(C)C)cs1)[C@H](CC(C)(C)C)CO2] | | | +++ |
| 701 | 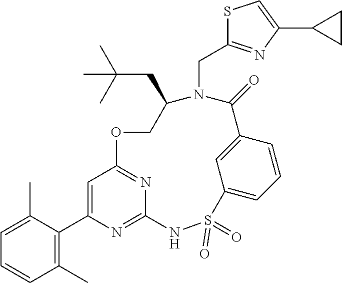 [SMILES: Cc1cccc(C)c1-c1cc2nc(n1)NS(=O)(=O)c1cccc(c1)C(=O)N(Cc1nc(C3CC3)cs1)[C@H](CC(C)(C)C)CO2] | | | +++ |
| 700 | 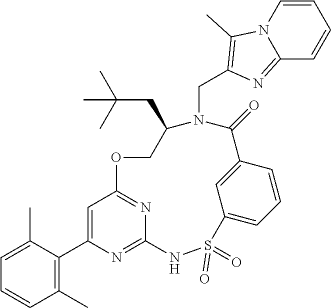 [SMILES: Cc1cccc(C)c1-c1cc2nc(n1)NS(=O)(=O)c1cccc(c1)C(=O)N(Cc1nc3ccccn3c1C)[C@H](CC(C)(C)C)CO2] | | | +++ |
| 699 | 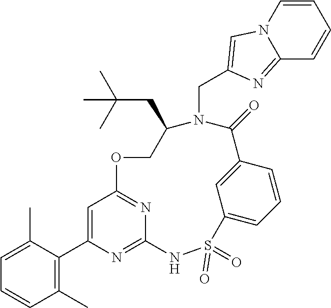 [SMILES: Cc1cccc(C)c1-c1cc2nc(n1)NS(=O)(=O)c1cccc(c1)C(=O)N(Cc1cn3ccccc3n1)[C@H](CC(C)(C)C)CO2] | +++ | +++ | |
| 269 | 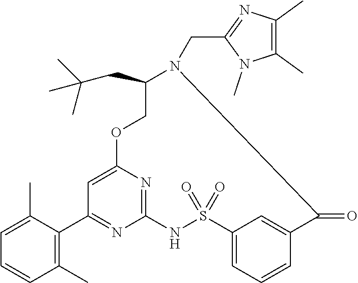 [SMILES: Cc1cccc(C)c1-c1cc2nc(n1)NS(=O)(=O)c1cccc(c1)C(=O)N(Cc1nc(C)c(C)n1C)[C@H](CC(C)(C)C)CO2] | | | +++ |
| 268 | 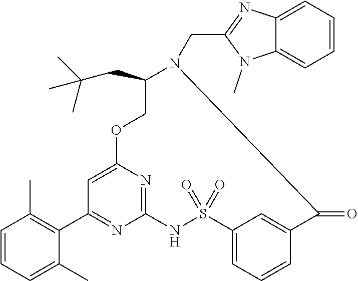 [SMILES: Cc1cccc(C)c1-c1cc2nc(n1)NS(=O)(=O)c1cccc(c1)C(=O)N(Cc1nc3ccccc3n1C)[C@H](CC(C)(C)C)CO2] | +++ | +++ | |
| 267 | 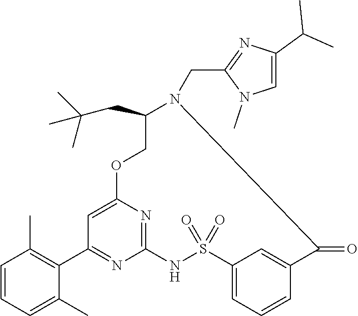 [SMILES: Cc1cccc(C)c1-c1cc2nc(n1)NS(=O)(=O)c1cccc(c1)C(=O)N(Cc1nc(C(C)C)cn1C)[C@H](CC(C)(C)C)CO2] | +++ | +++ | |
| 266 | 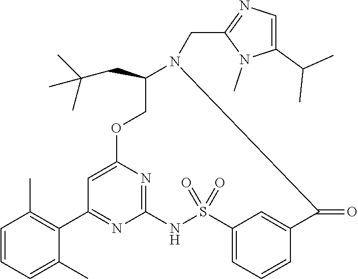 [SMILES: Cc1cccc(C)c1-c1cc2nc(n1)NS(=O)(=O)c1cccc(c1)C(=O)N(Cc1ncc(C(C)C)n1C)[C@H](CC(C)(C)C)CO2] | | | +++ |
| 265 | 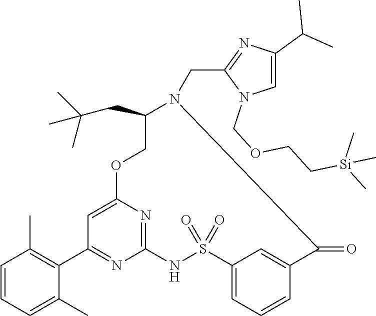 [SMILES: Cc1cccc(C)c1-c1cc2nc(n1)NS(=O)(=O)c1cccc(c1)C(=O)N(Cc1nc(C(C)C)cn1COCC[Si](C)(C)C)[C@H](CC(C)(C)C)CO2] | | | +++ |
| 264 | 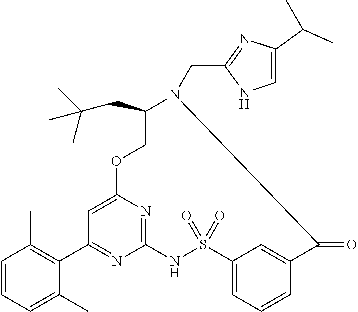 [SMILES: Cc1cccc(C)c1-c1cc2nc(n1)NS(=O)(=O)c1cccc(c1)C(=O)N(Cc1nc(C(C)C)c[nH]1)[C@H](CC(C)(C)C)CO2] | +++ | +++ | + |
| 577 | 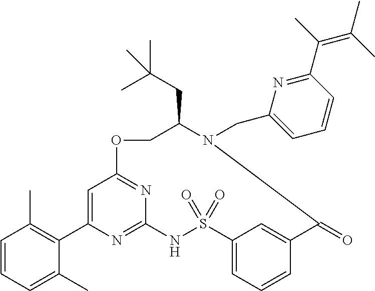 [SMILES: CC(C)=C(C)c1cccc(CN2C(=O)c3cccc(c3)S(=O)(=O)Nc3nc(cc(-c4c(C)cccc4C)n3)OC[C@H]2CC(C)(C)C)n1] | | | +++ |
| 575 | 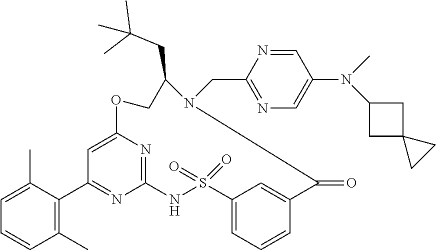 [SMILES: Cc1cccc(C)c1-c1cc2nc(n1)NS(=O)(=O)c1cccc(c1)C(=O)N(Cc1ncc(N(C)C3CC4(CC4)C3)cn1)[C@H](CC(C)(C)C)CO2] | +++ | +++ | |
| 574 | 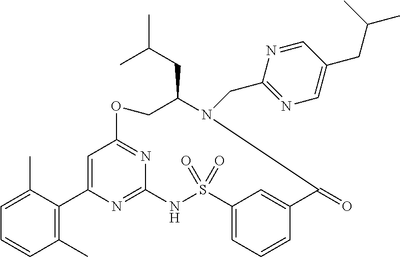 [SMILES: Cc1cccc(C)c1-c1cc2nc(n1)NS(=O)(=O)c1cccc(c1)C(=O)N(Cc1ncc(CC(C)C)cn1)[C@H](CC(C)C)CO2] | +++ | +++ | |
| 573 | 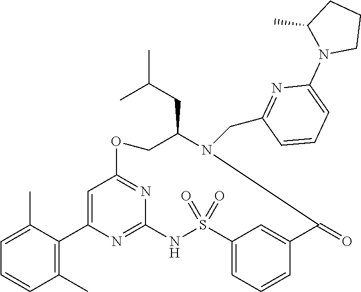 [SMILES: Cc1cccc(C)c1-c1cc2nc(n1)NS(=O)(=O)c1cccc(c1)C(=O)N(Cc1cccc(N3CCC[C@H]3C)n1)[C@H](CC(C)C)CO2] | +++ | +++ | |
| 327 | 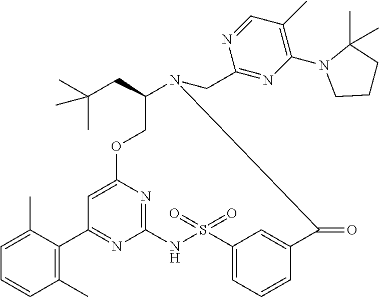 [SMILES: Cc1cnc(CN2C(=O)c3cccc(c3)S(=O)(=O)Nc3nc(cc(-c4c(C)cccc4C)n3)OC[C@H]2CC(C)(C)C)nc1N1CCCC1(C)C] | | | +++ |
| 453 | 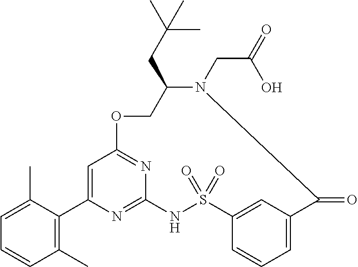 [SMILES: Cc1cccc(C)c1-c1cc2nc(n1)NS(=O)(=O)c1cccc(c1)C(=O)N(CC(=O)O)[C@H](CC(C)(C)C)CO2] | + | +++ | |
| 1008 | 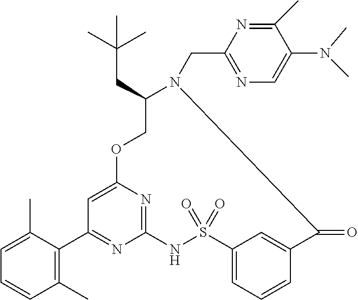 [SMILES: Cc1cccc(C)c1-c1cc2nc(n1)NS(=O)(=O)c1cccc(c1)C(=O)N(Cc1ncc(N(C)C)c(C)n1)[C@H](CC(C)(C)C)CO2] | | | +++ |
| 1007 | 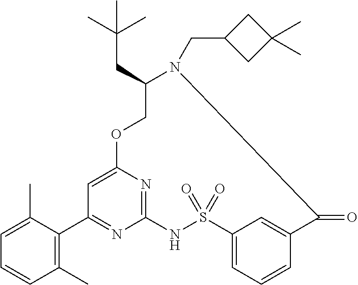 [SMILES: Cc1cccc(C)c1-c1cc2nc(n1)NS(=O)(=O)c1cccc(c1)C(=O)N(CC1CC(C)(C)C1)[C@H](CC(C)(C)C)CO2] | | | +++ |
| 450 | 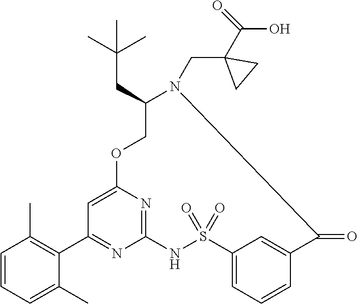 [SMILES: Cc1cccc(C)c1-c1cc2nc(n1)NS(=O)(=O)c1cccc(c1)C(=O)N(CC1(C(=O)O)CC1)[C@H](CC(C)(C)C)CO2] | +++ | | ++ |
| 451 | 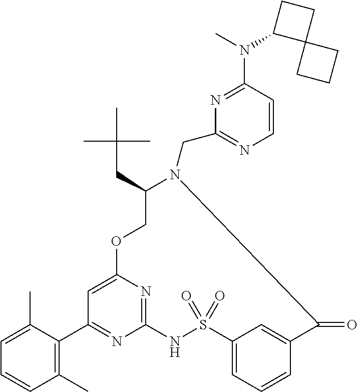 [SMILES: Cc1cccc(C)c1-c1cc2nc(n1)NS(=O)(=O)c1cccc(c1)C(=O)N(Cc1nccc(N(C)[C@@H]3CCC34CCC4)n1)[C@H](CC(C)(C)C)CO2] | +++ | +++ | |
| 452 | 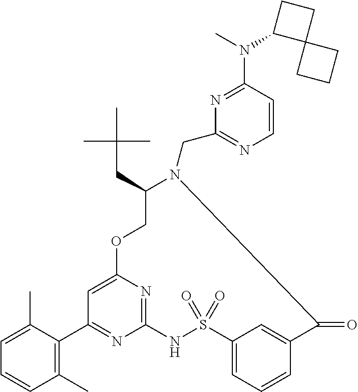 [SMILES: Cc1cccc(C)c1-c1cc2nc(n1)NS(=O)(=O)c1cccc(c1)C(=O)N(Cc1nccc(N(C)[C@@H]3CCC34CCC4)n1)[C@H](CC(C)(C)C)CO2] | +++ | +++ | |
| 879 | 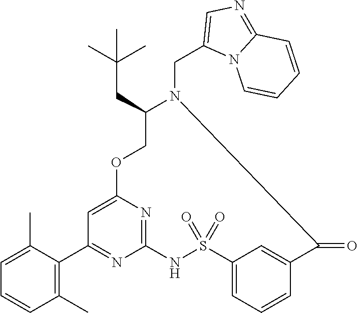 [SMILES: Cc1cccc(C)c1-c1cc2nc(n1)NS(=O)(=O)c1cccc(c1)C(=O)N(Cc1cnc3ccccn13)[C@H](CC(C)(C)C)CO2] | | | +++ |
| 878 | 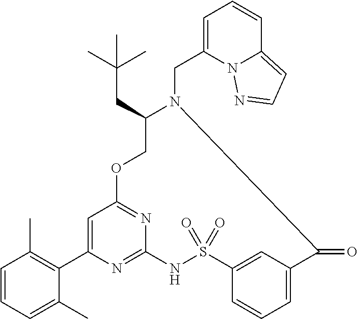 [SMILES: Cc1cccc(C)c1-c1cc2nc(n1)NS(=O)(=O)c1cccc(c1)C(=O)N(Cc1cccc3ccnn13)[C@H](CC(C)(C)C)CO2] | | | +++ |
| 232 | 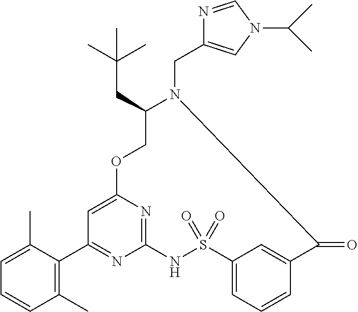 [SMILES: Cc1cccc(C)c1-c1cc2nc(n1)NS(=O)(=O)c1cccc(c1)C(=O)N(Cc1cn(C(C)C)cn1)[C@H](CC(C)(C)C)CO2] | | | +++ |
| 174 | 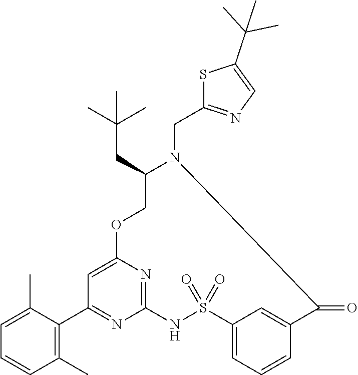 [SMILES: Cc1cccc(C)c1-c1cc2nc(n1)NS(=O)(=O)c1cccc(c1)C(=O)N(Cc1ncc(C(C)(C)C)s1)[C@H](CC(C)(C)C)CO2] | | | +++ |
| 167 | 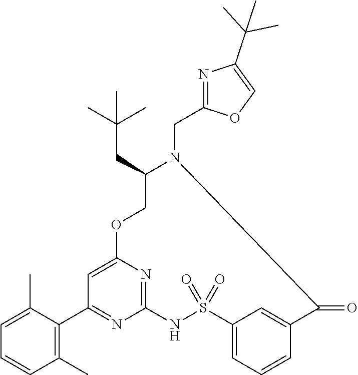 [SMILES: Cc1cccc(C)c1-c1cc2nc(n1)NS(=O)(=O)c1cccc(c1)C(=O)N(Cc1nc(C(C)(C)C)co1)[C@H](CC(C)(C)C)CO2] | | | +++ |
| 326 | 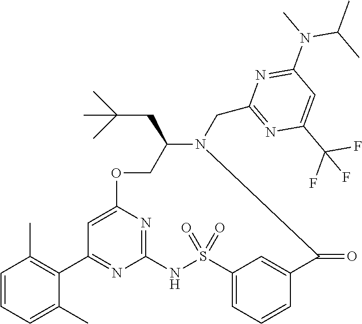 [SMILES: Cc1cccc(C)c1-c1cc2nc(n1)NS(=O)(=O)c1cccc(c1)C(=O)N(Cc1nc(N(C)C(C)C)cc(C(F)(F)F)n1)[C@H](CC(C)(C)C)CO2] | | | +++ |
| 568 | 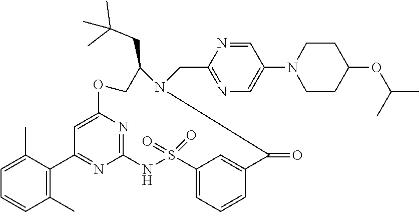 [SMILES: Cc1cccc(C)c1-c1cc2nc(n1)NS(=O)(=O)c1cccc(c1)C(=O)N(Cc1ncc(N3CCC(OC(C)C)CC3)cn1)[C@H](CC(C)(C)C)CO2] | +++ | +++ | |
| 569 | 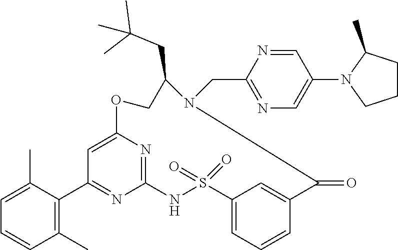 [SMILES: Cc1cccc(C)c1-c1cc2nc(n1)NS(=O)(=O)c1cccc(c1)C(=O)N(Cc1ncc(N3CCC[C@@H]3C)cn1)[C@H](CC(C)(C)C)CO2] | +++ | +++ | |
| 570 | 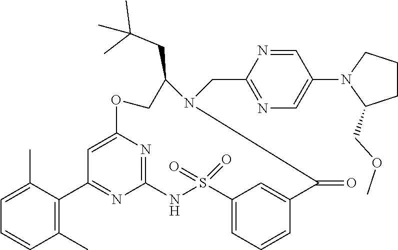 [SMILES: COC[C@H]1CCCN1c1cnc(CN2C(=O)c3cccc(c3)S(=O)(=O)Nc3nc(cc(-c4c(C)cccc4C)n3)OC[C@H]2CC(C)(C)C)nc1] | +++ | +++ | |
| 571 | 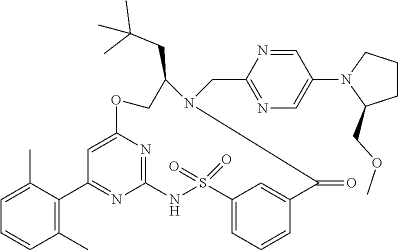 [SMILES: COC[C@@H]1CCCN1c1cnc(CN2C(=O)c3cccc(c3)S(=O)(=O)Nc3nc(cc(-c4c(C)cccc4C)n3)OC[C@H]2CC(C)(C)C)nc1] | +++ | +++ | |
| 572 | 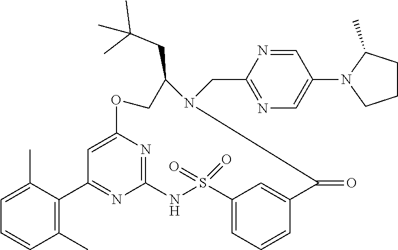 [SMILES: Cc1cccc(C)c1-c1cc2nc(n1)NS(=O)(=O)c1cccc(c1)C(=O)N(Cc1ncc(N3CCC[C@H]3C)cn1)[C@H](CC(C)(C)C)CO2] | +++ | +++ | |
| 707 | 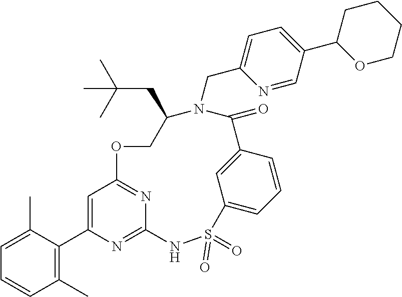 [SMILES: Cc1cccc(C)c1-c1cc2nc(n1)NS(=O)(=O)c1cccc(c1)C(=O)N(Cc1ccc(C3CCCCO3)cn1)[C@H](CC(C)(C)C)CO2] | +++ | +++ | |
| 706 | 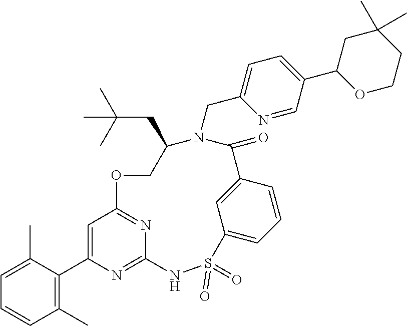 [SMILES: Cc1cccc(C)c1-c1cc2nc(n1)NS(=O)(=O)c1cccc(c1)C(=O)N(Cc1ccc(C3CC(C)(C)CCO3)cn1)[C@H](CC(C)(C)C)CO2] | +++ | +++ | |
| 698 | 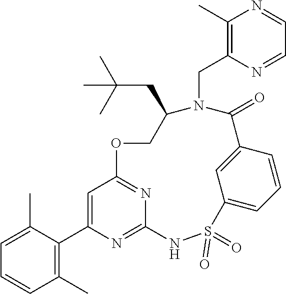 [SMILES: Cc1cccc(C)c1-c1cc2nc(n1)NS(=O)(=O)c1cccc(c1)C(=O)N(Cc1nccnc1C)[C@H](CC(C)(C)C)CO2] | | | +++ |
| 697 | 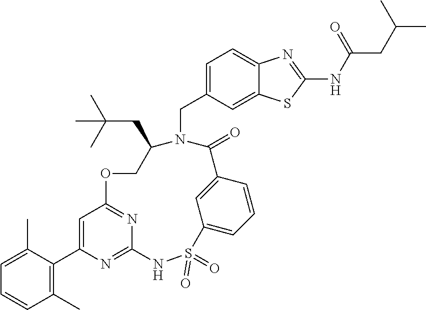 [SMILES: Cc1cccc(C)c1-c1cc2nc(n1)NS(=O)(=O)c1cccc(c1)C(=O)N(Cc1ccc3nc(NC(=O)CC(C)C)sc3c1)[C@H](CC(C)(C)C)CO2] | | | +++ |
| 449 | 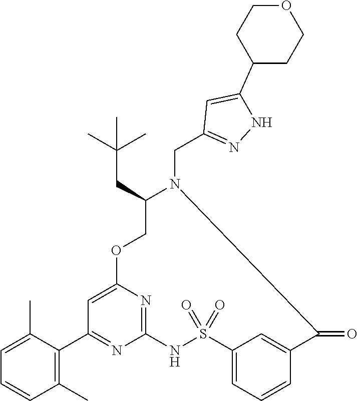 [SMILES: Cc1cccc(C)c1-c1cc2nc(n1)NS(=O)(=O)c1cccc(c1)C(=O)N(Cc1cc(C3CCOCC3)[nH]n1)[C@H](CC(C)(C)C)CO2] | | | +++ |
| 448 | 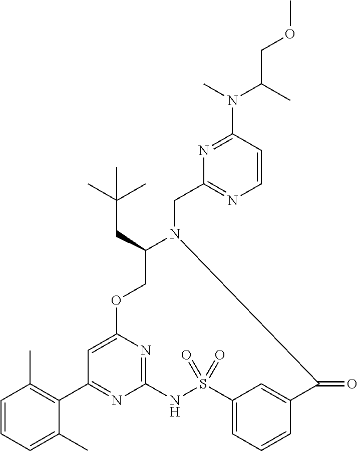 [SMILES: COCC(C)N(C)c1ccnc(CN2C(=O)c3cccc(c3)S(=O)(=O)Nc3nc(cc(-c4c(C)cccc4C)n3)OC[C@H]2CC(C)(C)C)n1] | | | +++ |
| 1064 | 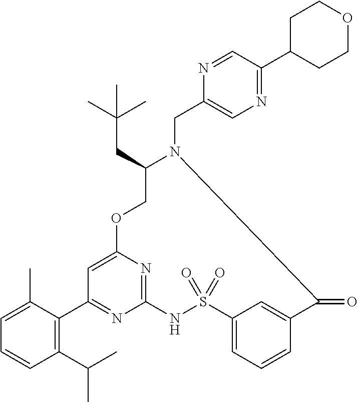 [SMILES: Cc1cccc(C(C)C)c1-c1cc2nc(n1)NS(=O)(=O)c1cccc(c1)C(=O)N(Cc1cnc(C3CCOCC3)cn1)[C@H](CC(C)(C)C)CO2] | +++ | +++ | |
| 1006 | 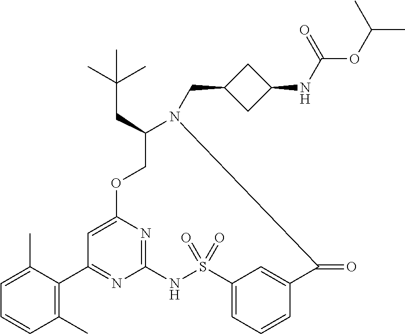 [SMILES: Cc1cccc(C)c1-c1cc2nc(n1)NS(=O)(=O)c1cccc(c1)C(=O)N(C[C@H]1C[C@@H](NC(=O)OC(C)C)C1)[C@H](CC(C)(C)C)CO2] | | | +++ |
| 1005 | 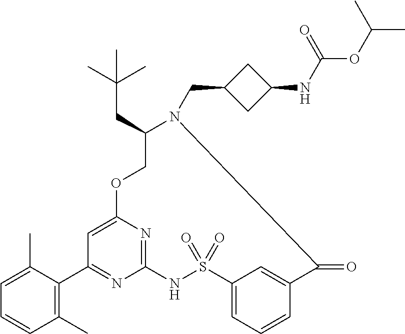 [SMILES: Cc1cccc(C)c1-c1cc2nc(n1)NS(=O)(=O)c1cccc(c1)C(=O)N(C[C@H]1C[C@@H](NC(=O)OC(C)C)C1)[C@H](CC(C)(C)C)CO2] | +++ | +++ | |
| 526 | 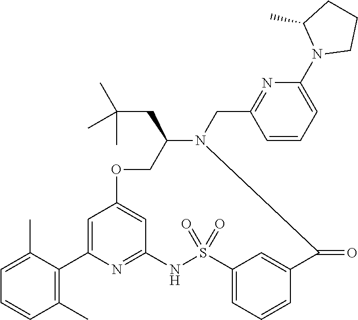 [SMILES: Cc1cccc(C)c1-c1cc2cc(n1)NS(=O)(=O)c1cccc(c1)C(=O)N(Cc1cccc(N3CCC[C@H]3C)n1)[C@H](CC(C)(C)C)CO2] | +++ | +++ | |
| 166 | 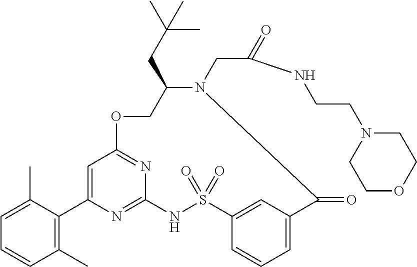 [SMILES: Cc1cccc(C)c1-c1cc2nc(n1)NS(=O)(=O)c1cccc(c1)C(=O)N(CC(=O)NCCN1CCOCC1)[C@H](CC(C)(C)C)CO2] | | | +++ |
| 563 | 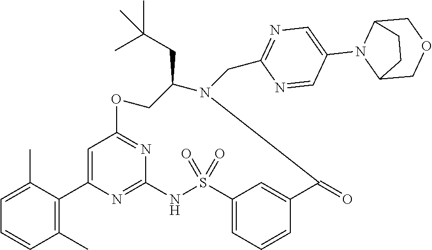 [SMILES: Cc1cccc(C)c1-c1cc2nc(n1)NS(=O)(=O)c1cccc(c1)C(=O)N(Cc1ncc(N3C4CCC3COC4)cn1)[C@H](CC(C)(C)C)CO2] | +++ | +++ | |
| 564 | 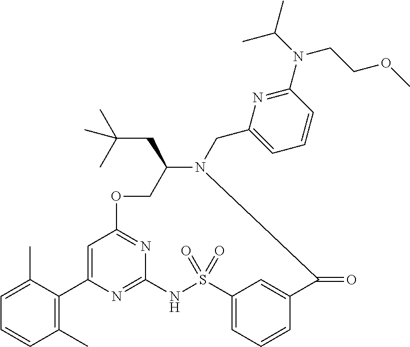 [SMILES: COCCN(c1cccc(CN2C(=O)c3cccc(c3)S(=O)(=O)Nc3nc(cc(-c4c(C)cccc4C)n3)OC[C@H]2CC(C)(C)C)n1)C(C)C] | +++ | +++ | |
| 565 | 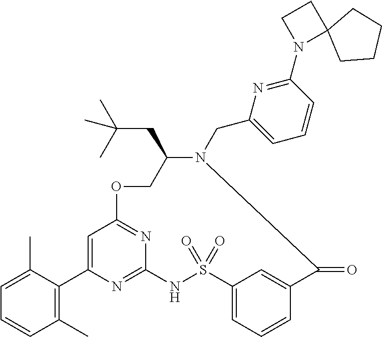 [SMILES: Cc1cccc(C)c1-c1cc2nc(n1)NS(=O)(=O)c1cccc(c1)C(=O)N(Cc1cccc(N3CCC34CCCC4)n1)[C@H](CC(C)(C)C)CO2] | +++ | +++ | |
| 566 | 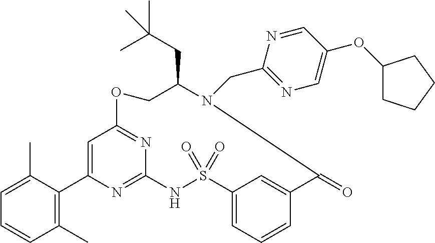 [SMILES: Cc1cccc(C)c1-c1cc2nc(n1)NS(=O)(=O)c1cccc(c1)C(=O)N(Cc1ncc(OC3CCCC3)cn1)[C@H](CC(C)(C)C)CO2] | +++ | +++ | |
| 567 | 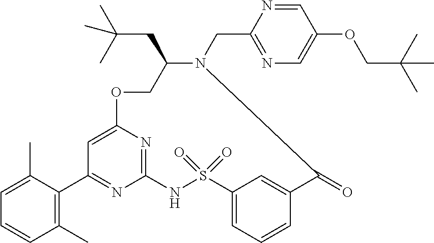 [SMILES: Cc1cccc(C)c1-c1cc2nc(n1)NS(=O)(=O)c1cccc(c1)C(=O)N(Cc1ncc(OCC(C)(C)C)cn1)[C@H](CC(C)(C)C)CO2] | +++ | +++ | |
| 525 | 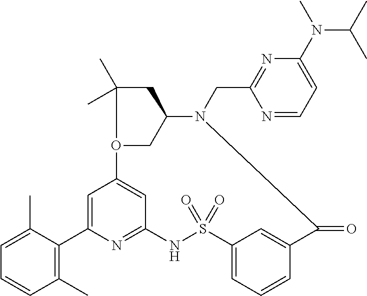 | +++ | +++ | |
| 243 | 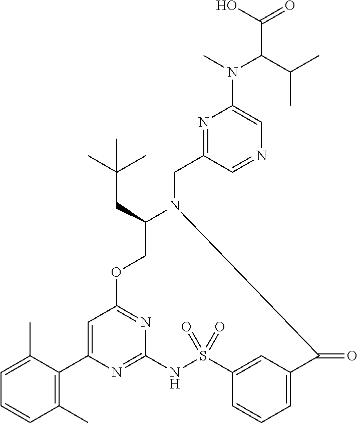 [SMILES: Cc1cccc(C)c1-c1cc2nc(n1)NS(=O)(=O)c1cccc(c1)C(=O)N(Cc1cncc(N(C)C(C(=O)O)C(C)C)n1)[C@H](CC(C)(C)C)CO2] | | | +++ |
| 447 | 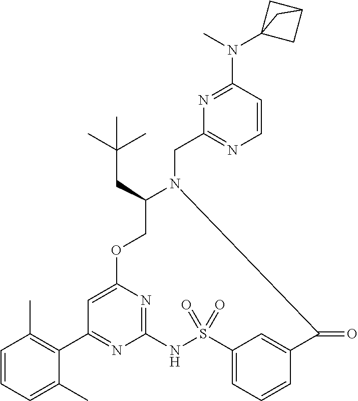 [SMILES: Cc1cccc(C)c1-c1cc2nc(n1)NS(=O)(=O)c1cccc(c1)C(=O)N(Cc1nccc(N(C)C34CC(C3)C4)n1)[C@H](CC(C)(C)C)CO2] | | | +++ |
| 446 | 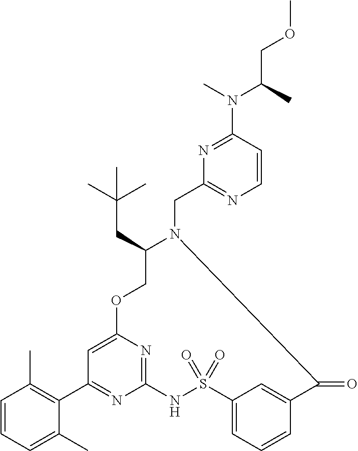 [SMILES: COC[C@@H](C)N(C)c1ccnc(CN2C(=O)c3cccc(c3)S(=O)(=O)Nc3nc(cc(-c4c(C)cccc4C)n3)OC[C@H]2CC(C)(C)C)n1] | +++ | +++ | |
| 441 | 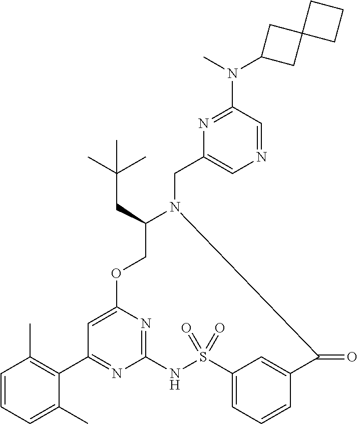 [SMILES: Cc1cccc(C)c1-c1cc2nc(n1)NS(=O)(=O)c1cccc(c1)C(=O)N(Cc1cncc(N(C)C3CC4(CCC4)C3)n1)[C@H](CC(C)(C)C)CO2] | +++ | +++ | |
| 524 | 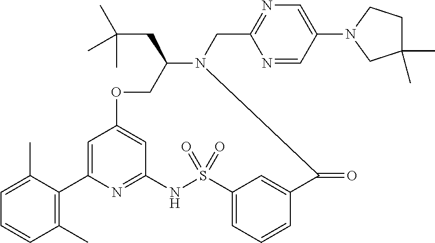 [SMILES: Cc1cccc(C)c1-c1cc2cc(n1)NS(=O)(=O)c1cccc(c1)C(=O)N(Cc1ncc(N3CCC(C)(C)C3)cn1)[C@H](CC(C)(C)C)CO2] | +++ | +++ | |
| 696 | 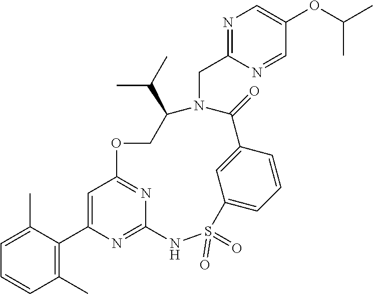 [SMILES: Cc1cccc(C)c1-c1cc2nc(n1)NS(=O)(=O)c1cccc(c1)C(=O)N(Cc1ncc(OC(C)C)cn1)[C@H](C(C)C)CO2] | +++ | + | +++ |
| 695 | 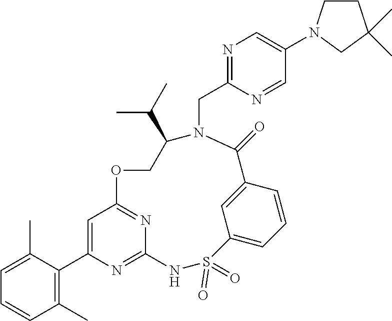 [SMILES: Cc1cccc(C)c1-c1cc2nc(n1)NS(=O)(=O)c1cccc(c1)C(=O)N(Cc1ncc(N3CCC(C)(C)C3)cn1)[C@H](C(C)C)CO2] | +++ | + | +++ |
| 694 | 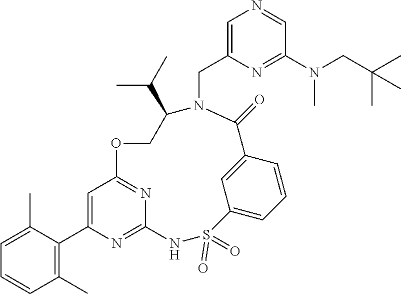 [SMILES: Cc1cccc(C)c1-c1cc2nc(n1)NS(=O)(=O)c1cccc(c1)C(=O)N(Cc1cncc(N(C)CC(C)(C)C)n1)[C@H](C(C)C)CO2] | +++ | +++ | |
| 230 | 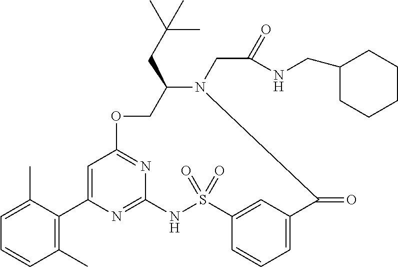 [SMILES: Cc1cccc(C)c1-c1cc2nc(n1)NS(=O)(=O)c1cccc(c1)C(=O)N(CC(=O)NCC1CCCCC1)[C@H](CC(C)(C)C)CO2] | +++ | +++ | |
| 231 | 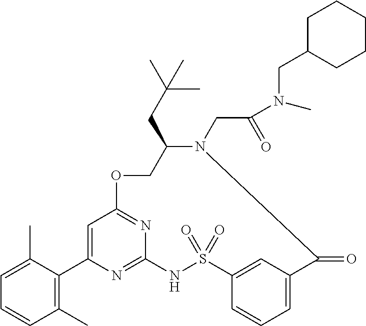 [SMILES: Cc1cccc(C)c1-c1cc2nc(n1)NS(=O)(=O)c1cccc(c1)C(=O)N(CC(=O)N(C)CC1CCCCC1)[C@H](CC(C)(C)C)CO2] | +++ | +++ | |
| 228 | 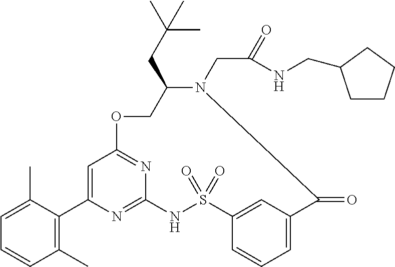 [SMILES: Cc1cccc(C)c1-c1cc2nc(n1)NS(=O)(=O)c1cccc(c1)C(=O)N(CC(=O)NCC1CCCC1)[C@H](CC(C)(C)C)CO2] | | | ++ |
| 229 | 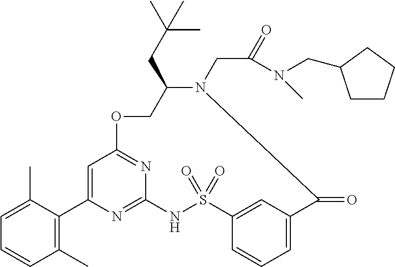 [SMILES: Cc1cccc(C)c1-c1cc2nc(n1)NS(=O)(=O)c1cccc(c1)C(=O)N(CC(=O)N(C)CC1CCCC1)[C@H](CC(C)(C)C)CO2] | | | +++ |
| 877 | 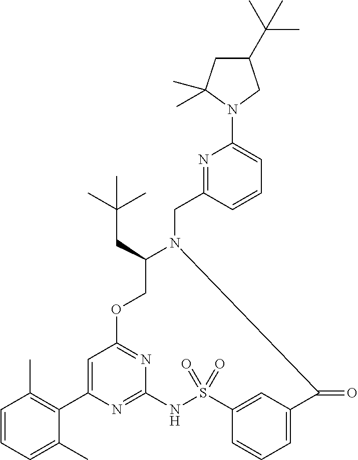 [SMILES: Cc1cccc(C)c1-c1cc2nc(n1)NS(=O)(=O)c1cccc(c1)C(=O)N(Cc1cccc(N3CC(C(C)(C)C)CC3(C)C)n1)[C@H](CC(C)(C)C)CO2] | | | +++ |
| 876 | 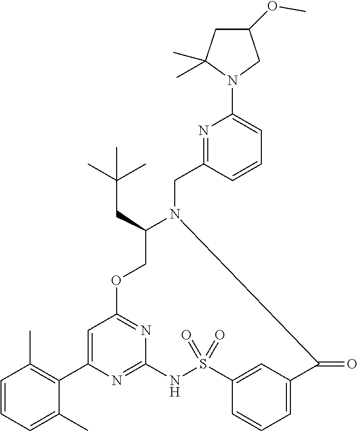 [SMILES: COC1CN(c2cccc(CN3C(=O)c4cccc(c4)S(=O)(=O)Nc4nc(cc(-c5c(C)cccc5C)n4)OC[C@H]3CC(C)(C)C)n2)C(C)(C)C1] | +++ | +++ | |
| 875 | 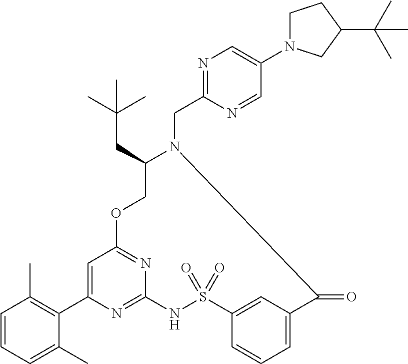 [SMILES: Cc1cccc(C)c1-c1cc2nc(n1)NS(=O)(=O)c1cccc(c1)C(=O)N(Cc1ncc(N3CCC(C(C)(C)C)C3)cn1)[C@H](CC(C)(C)C)CO2] | +++ | +++ | |
| 874 | 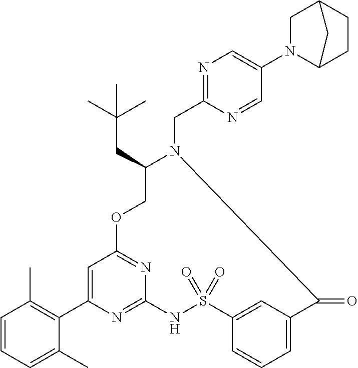 [SMILES: Cc1cccc(C)c1-c1cc2nc(n1)NS(=O)(=O)c1cccc(c1)C(=O)N(Cc1ncc(N3CC4CCC3C4)cn1)[C@H](CC(C)(C)C)CO2] | +++ | +++ | |
| 871 | 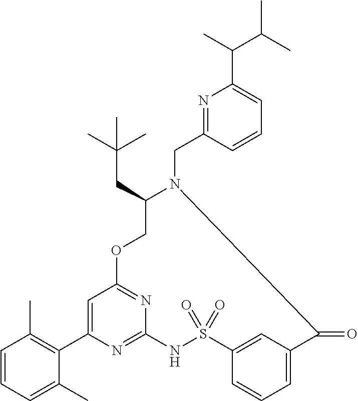 [SMILES: Cc1cccc(C)c1-c1cc2nc(n1)NS(=O)(=O)c1cccc(c1)C(=O)N(Cc1cccc(C(C)C(C)C)n1)[C@H](CC(C)(C)C)CO2] | +++ | +++ | |
| 872 | 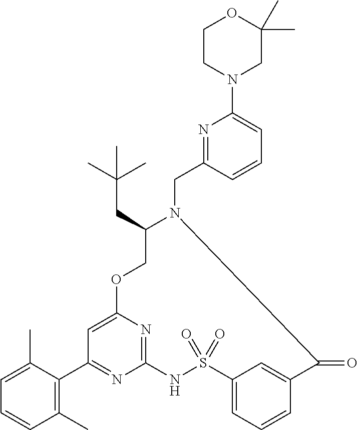 [SMILES: Cc1cccc(C)c1-c1cc2nc(n1)NS(=O)(=O)c1cccc(c1)C(=O)N(Cc1cccc(N3CCOC(C)(C)C3)n1)[C@H](CC(C)(C)C)CO2] | +++ | +++ | |
| 523 | 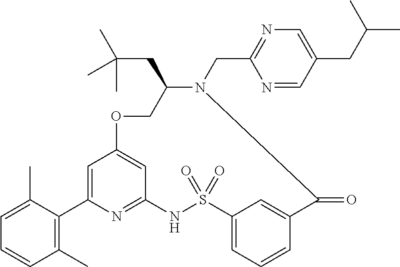 [SMILES: Cc1cccc(C)c1-c1cc2cc(n1)NS(=O)(=O)c1cccc(c1)C(=O)N(Cc1ncc(CC(C)C)cn1)[C@H](CC(C)(C)C)CO2] | | | +++ |
| 242 | 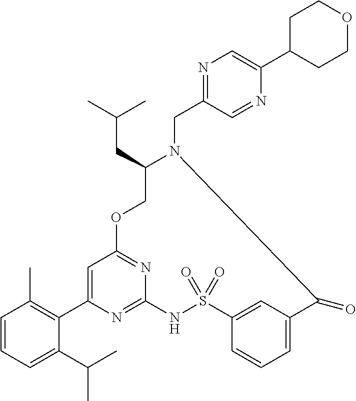 [SMILES: Cc1cccc(C(C)C)c1-c1cc2nc(n1)NS(=O)(=O)c1cccc(c1)C(=O)N(Cc1cnc(C3CCOCC3)cn1)[C@H](CC(C)C)CO2] | | | +++ |
| 444 | 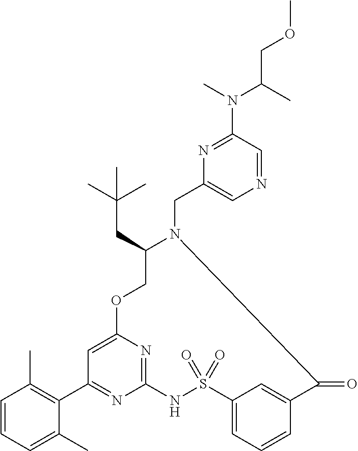 [SMILES: COCC(C)N(C)c1cncc(CN2C(=O)c3cccc(c3)S(=O)(=O)Nc3nc(cc(-c4c(C)cccc4C)n3)OC[C@H]2CC(C)(C)C)n1] | +++ | +++ | |
| 442 | 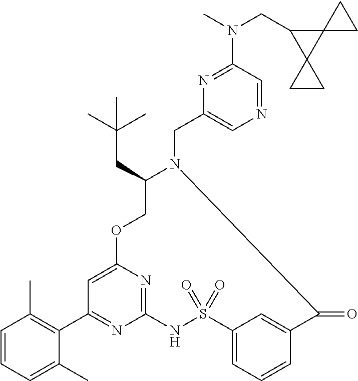 [SMILES: Cc1cccc(C)c1-c1cc2nc(n1)NS(=O)(=O)c1cccc(c1)C(=O)N(Cc1cncc(N(C)CC3C4(CC4)C34CC4)n1)[C@H](CC(C)(C)C)CO2] | +++ | +++ | |
| 324 | 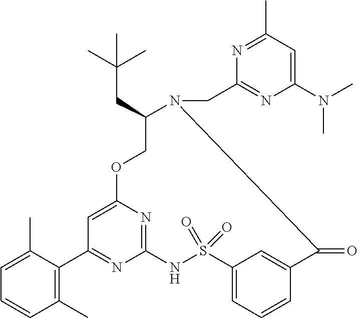 [SMILES: Cc1cc(N(C)C)nc(CN2C(=O)c3cccc(c3)S(=O)(=O)Nc3nc(cc(-c4c(C)cccc4C)n3)OC[C@H]2CC(C)(C)C)n1] | | | +++ |
| 325 | 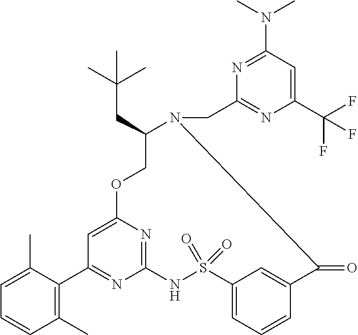 [SMILES: Cc1cccc(C)c1-c1cc2nc(n1)NS(=O)(=O)c1cccc(c1)C(=O)N(Cc1nc(N(C)C)cc(C(F)(F)F)n1)[C@H](CC(C)(C)C)CO2] | | | +++ |
| 562 | 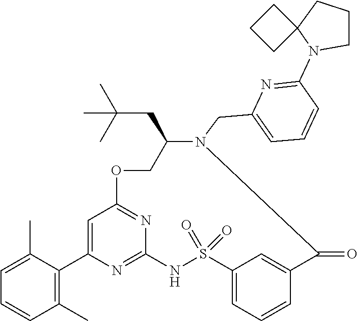 [SMILES: Cc1cccc(C)c1-c1cc2nc(n1)NS(=O)(=O)c1cccc(c1)C(=O)N(Cc1cccc(N3CCCC34CCC4)n1)[C@H](CC(C)(C)C)CO2] | +++ | +++ | |
| 561 | 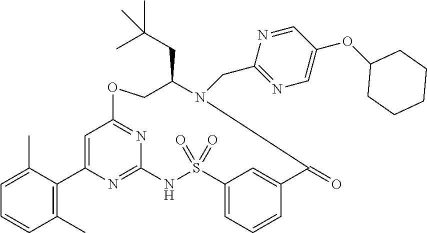 [SMILES: Cc1cccc(C)c1-c1cc2nc(n1)NS(=O)(=O)c1cccc(c1)C(=O)N(Cc1ncc(OC3CCCCC3)cn1)[C@H](CC(C)(C)C)CO2] | +++ | +++ | +++ |
| 560 | 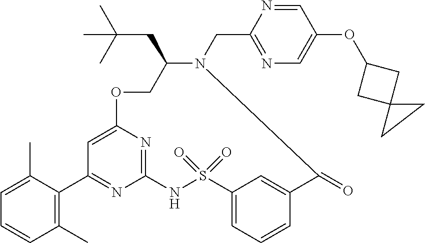 [SMILES: Cc1cccc(C)c1-c1cc2nc(n1)NS(=O)(=O)c1cccc(c1)C(=O)N(Cc1ncc(OC3CC4(CC4)C3)cn1)[C@H](CC(C)(C)C)CO2] | +++ | +++ | |
| 559 | 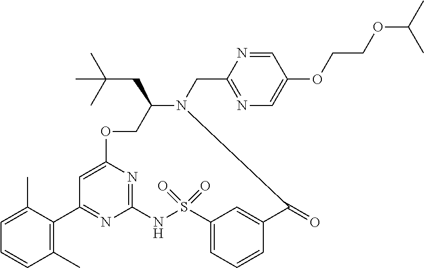 [SMILES: Cc1cccc(C)c1-c1cc2nc(n1)NS(=O)(=O)c1cccc(c1)C(=O)N(Cc1ncc(OCCOC(C)C)cn1)[C@H](CC(C)(C)C)CO2] | +++ | +++ | |
| 558 | 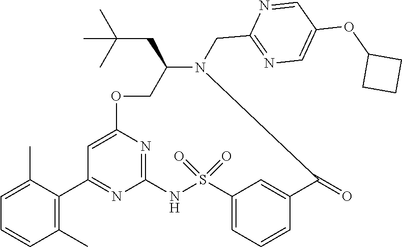 [SMILES: Cc1cccc(C)c1-c1cc2nc(n1)NS(=O)(=O)c1cccc(c1)C(=O)N(Cc1ncc(OC3CCC3)cn1)[C@H](CC(C)(C)C)CO2] | +++ | +++ | |
| 557 | 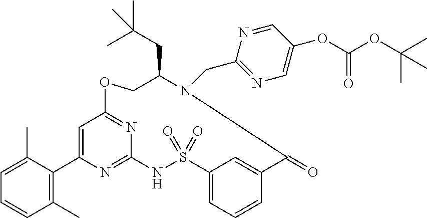 [SMILES: Cc1cccc(C)c1-c1cc2nc(n1)NS(=O)(=O)c1cccc(c1)C(=O)N(Cc1ncc(OC(=O)OC(C)(C)C)cn1)[C@H](CC(C)(C)C)CO2] | | | +++ |
| 556 | 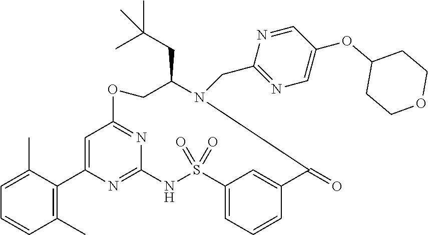 [SMILES: Cc1cccc(C)c1-c1cc2nc(n1)NS(=O)(=O)c1cccc(c1)C(=O)N(Cc1ncc(OC3CCOCC3)cn1)[C@H](CC(C)(C)C)CO2] | +++ | +++ | |
| 555 | 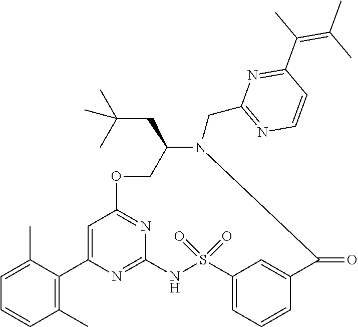 [SMILES: CC(C)=C(C)c1ccnc(CN2C(=O)c3cccc(c3)S(=O)(=O)Nc3nc(cc(-c4c(C)cccc4C)n3)OC[C@H]2CC(C)(C)C)n1] | | | +++ |
| 554 | 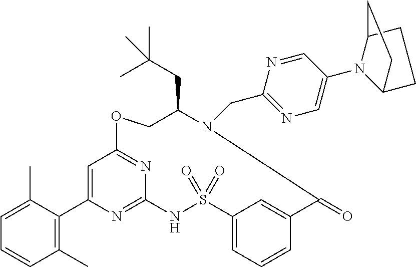 [SMILES: Cc1cccc(C)c1-c1cc2nc(n1)NS(=O)(=O)c1cccc(c1)C(=O)N(Cc1ncc(N3C4CCC3CC4)cn1)[C@H](CC(C)(C)C)CO2] | +++ | +++ | |
| 553 | 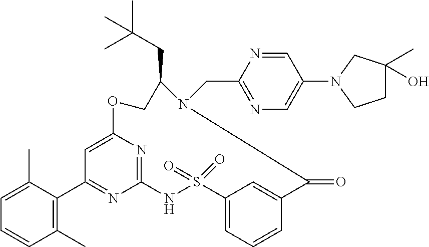 [SMILES: Cc1cccc(C)c1-c1cc2nc(n1)NS(=O)(=O)c1cccc(c1)C(=O)N(Cc1ncc(N3CCC(C)(O)C3)cn1)[C@H](CC(C)(C)C)CO2] | | | +++ |
| 263 | 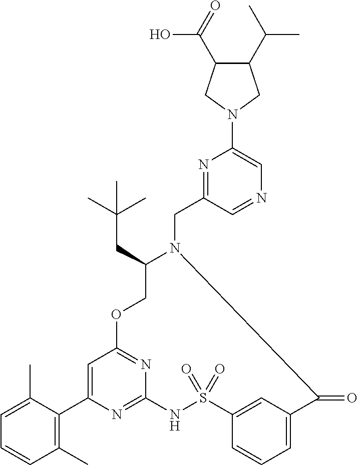 [SMILES: Cc1cccc(C)c1-c1cc2nc(n1)NS(=O)(=O)c1cccc(c1)C(=O)N(Cc1cncc(N3CC(C(=O)O)C(C(C)C)C3)n1)[C@H](CC(C)(C)C)CO2] | | | ++ |
| 323 | 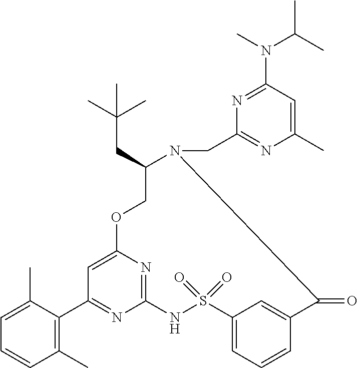 [SMILES: Cc1cc(N(C)C(C)C)nc(CN2C(=O)c3cccc(c3)S(=O)(=O)Nc3nc(cc(-c4c(C)cccc4C)n3)OC[C@H]2CC(C)(C)C)n1] | | | +++ |
| 322 | 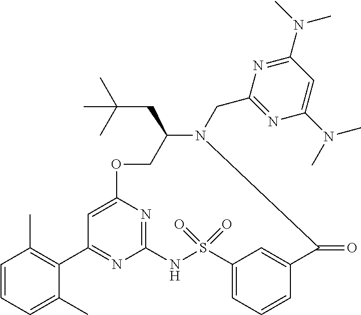 [SMILES: Cc1cccc(C)c1-c1cc2nc(n1)NS(=O)(=O)c1cccc(c1)C(=O)N(Cc1nc(N(C)C)cc(N(C)C)n1)[C@H](CC(C)(C)C)CO2] | +++ | +++ | |
| 280 | 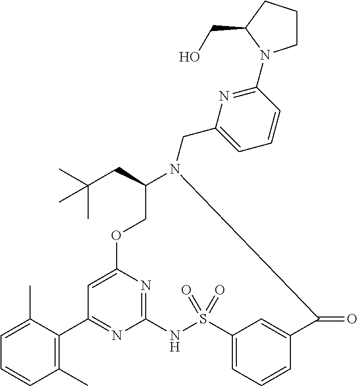 [SMILES: Cc1cccc(C)c1-c1cc2nc(n1)NS(=O)(=O)c1cccc(c1)C(=O)N(Cc1cccc(N3CCC[C@@H]3CO)n1)[C@H](CC(C)(C)C)CO2] | +++ | +++ | |
| 279 | 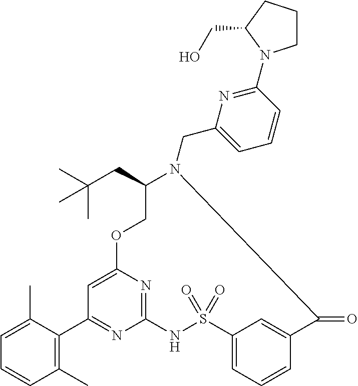 [SMILES: Cc1cccc(C)c1-c1cc2nc(n1)NS(=O)(=O)c1cccc(c1)C(=O)N(Cc1cccc(N3CCC[C@H]3CO)n1)[C@H](CC(C)(C)C)CO2] | +++ | +++ | |
| 262 | 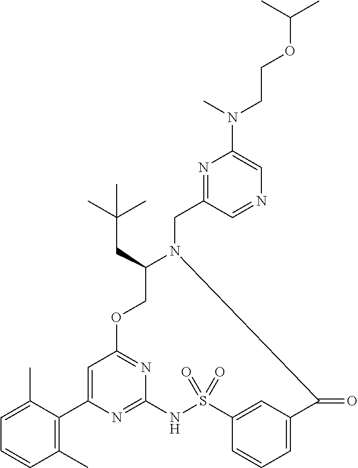 [SMILES: Cc1cccc(C)c1-c1cc2nc(n1)NS(=O)(=O)c1cccc(c1)C(=O)N(Cc1cncc(N(C)CCOC(C)C)n1)[C@H](CC(C)(C)C)CO2] | +++ | +++ | |
| 241 | 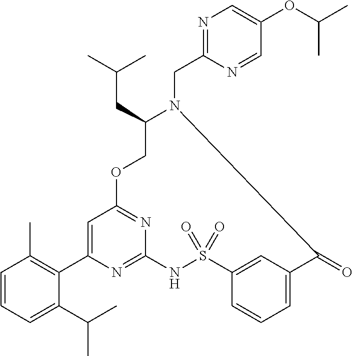 [SMILES: Cc1cccc(C(C)C)c1-c1cc2nc(n1)NS(=O)(=O)c1cccc(c1)C(=O)N(Cc1ncc(OC(C)C)cn1)[C@H](CC(C)C)CO2] | | | +++ |
| 215 | 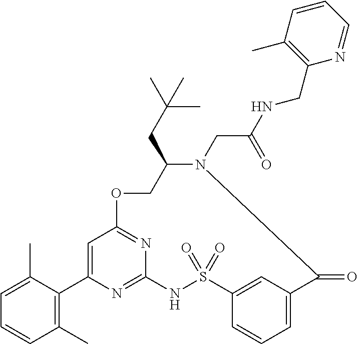 [SMILES: Cc1cccnc1CNC(=O)CN1C(=O)c2cccc(c2)S(=O)(=O)Nc2nc(cc(-c3c(C)cccc3C)n2)OC[C@H]1CC(C)(C)C] | | | +++ |
| 216 | 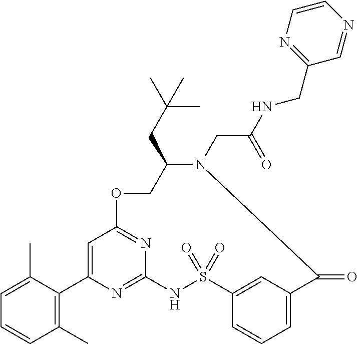 [SMILES: Cc1cccc(C)c1-c1cc2nc(n1)NS(=O)(=O)c1cccc(c1)C(=O)N(CC(=O)NCc1cnccn1)[C@H](CC(C)(C)C)CO2] | | | +++ |
| 217 | 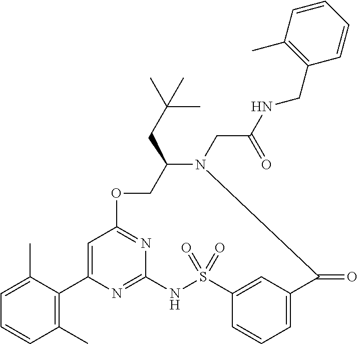 [SMILES: Cc1ccccc1CNC(=O)CN1C(=O)c2cccc(c2)S(=O)(=O)Nc2nc(cc(-c3c(C)cccc3C)n2)OC[C@H]1CC(C)(C)C] | | | +++ |
| 218 | 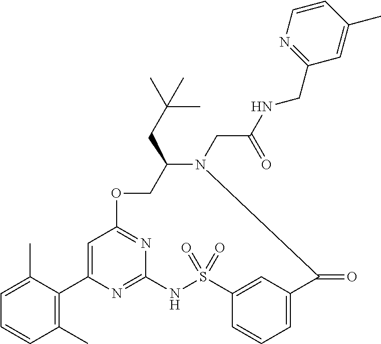 [SMILES: Cc1ccnc(CNC(=O)CN2C(=O)c3cccc(c3)S(=O)(=O)Nc3nc(cc(-c4c(C)cccc4C)n3)OC[C@H]2CC(C)(C)C)c1] | | | +++ |
| 224 | 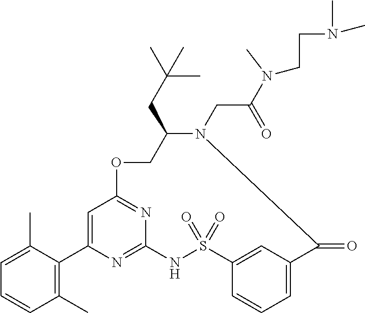 [SMILES: Cc1cccc(C)c1-c1cc2nc(n1)NS(=O)(=O)c1cccc(c1)C(=O)N(CC(=O)N(C)CCN(C)C)[C@H](CC(C)(C)C)CO2] | | | +++ |
| 522 | 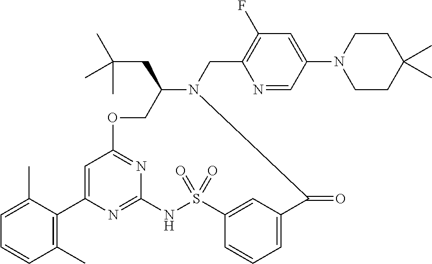 [SMILES: Cc1cccc(C)c1-c1cc2nc(n1)NS(=O)(=O)c1cccc(c1)C(=O)N(Cc1ncc(N3CCC(C)(C)CC3)cc1F)[C@H](CC(C)(C)C)CO2] | +++ | +++ | |
| 521 | 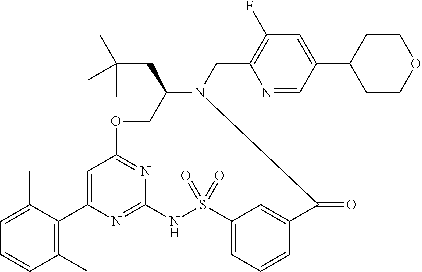 [SMILES: Cc1cccc(C)c1-c1cc2nc(n1)NS(=O)(=O)c1cccc(c1)C(=O)N(Cc1ncc(C3CCOCC3)cc1F)[C@H](CC(C)(C)C)CO2] | | | +++ |
| 873 | 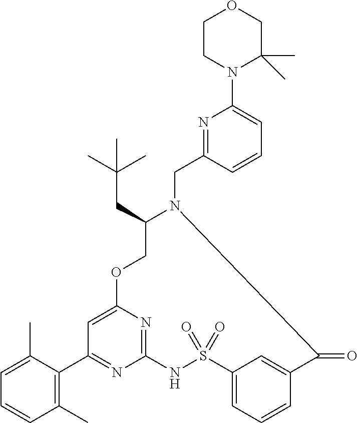 [SMILES: Cc1cccc(C)c1-c1cc2nc(n1)NS(=O)(=O)c1cccc(c1)C(=O)N(Cc1cccc(N3CCOCC3(C)C)n1)[C@H](CC(C)(C)C)CO2] | | | |
| 445 | 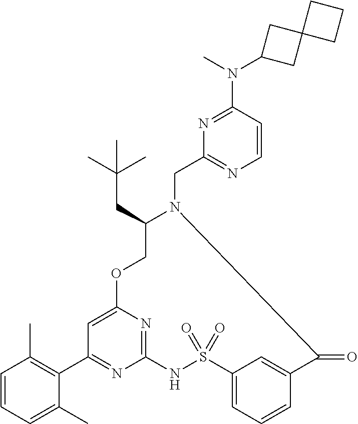 [SMILES: Cc1cccc(C)c1-c1cc2nc(n1)NS(=O)(=O)c1cccc(c1)C(=O)N(Cc1nccc(N(C)C3CC4(CCC4)C3)n1)[C@H](CC(C)(C)C)CO2] | +++ | +++ | |
| 443 | 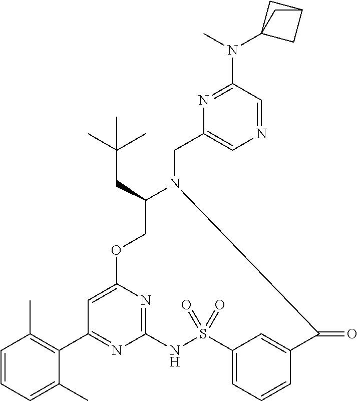 [SMILES: Cc1cccc(C)c1-c1cc2nc(n1)NS(=O)(=O)c1cccc(c1)C(=O)N(Cc1cncc(N(C)C34CC(C3)C4)n1)[C@H](CC(C)(C)C)CO2] | +++ | +++ | |
| 437 | 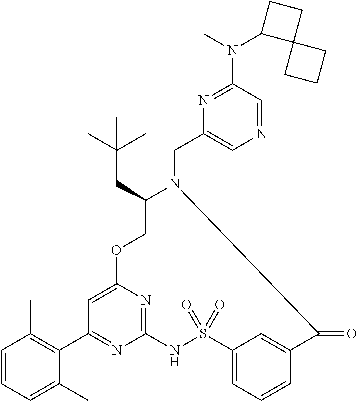 [SMILES: Cc1cccc(C)c1-c1cc2nc(n1)NS(=O)(=O)c1cccc(c1)C(=O)N(Cc1cncc(N(C)C3CCC34CCC4)n1)[C@H](CC(C)(C)C)CO2] | +++ | +++ | |
| 438 | 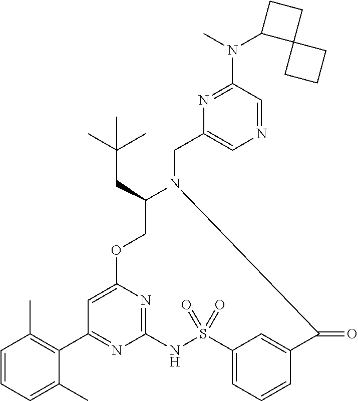 [SMILES: Cc1cccc(C)c1-c1cc2nc(n1)NS(=O)(=O)c1cccc(c1)C(=O)N(Cc1cncc(N(C)C3CCC34CCC4)n1)[C@H](CC(C)(C)C)CO2] | +++ | +++ | |
| 221 | 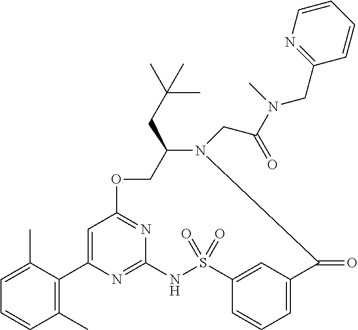 [SMILES: Cc1cccc(C)c1-c1cc2nc(n1)NS(=O)(=O)c1cccc(c1)C(=O)N(CC(=O)N(C)Cc1ccccn1)[C@H](CC(C)(C)C)CO2] | +++ | +++ | |
| 222 | 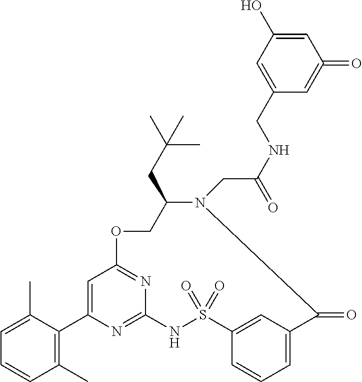 | | | +++ |
| 440 | 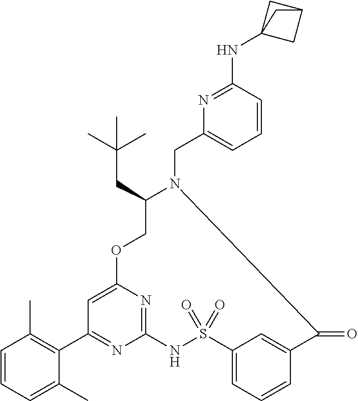 [SMILES: Cc1cccc(C)c1-c1cc2nc(n1)NS(=O)(=O)c1cccc(c1)C(=O)N(Cc1cccc(NC34CC(C3)C4)n1)[C@H](CC(C)(C)C)CO2] | +++ | +++ | |
| 439 | 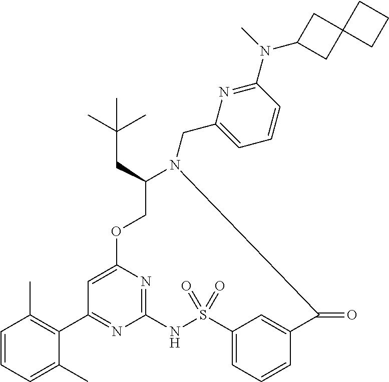 [SMILES: Cc1cccc(C)c1-c1cc2nc(n1)NS(=O)(=O)c1cccc(c1)C(=O)N(Cc1cccc(N(C)C3CC4(CCC4)C3)n1)[C@H](CC(C)(C)C)CO2] | +++ | +++ | |
| 240 | 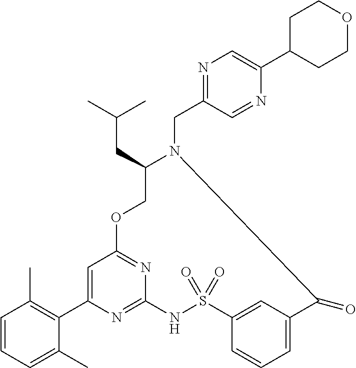 [SMILES: Cc1cccc(C)c1-c1cc2nc(n1)NS(=O)(=O)c1cccc(c1)C(=O)N(Cc1cnc(C3CCOCC3)cn1)[C@H](CC(C)C)CO2] | +++ | +++ | |
| 156 | 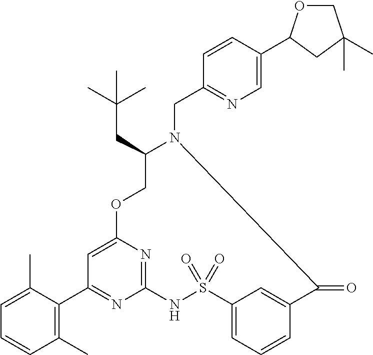 [SMILES: Cc1cccc(C)c1-c1cc2nc(n1)NS(=O)(=O)c1cccc(c1)C(=O)N(Cc1ccc(C3CC(C)(C)CO3)cn1)[C@H](CC(C)(C)C)CO2] | +++ | +++ | |
| 260 | 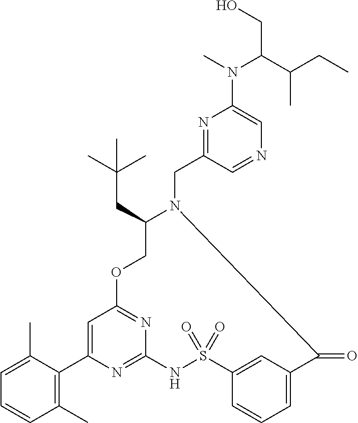 [SMILES: CCC(C)C(CO)N(C)c1cncc(CN2C(=O)c3cccc(c3)S(=O)(=O)Nc3nc(cc(-c4c(C)cccc4C)n3)OC[C@H]2CC(C)(C)C)n1] | +++ | +++ | |
| 549 | 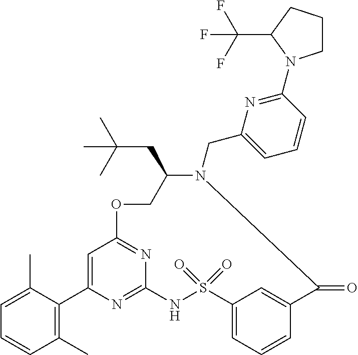 [SMILES: Cc1cccc(C)c1-c1cc2nc(n1)NS(=O)(=O)c1cccc(c1)C(=O)N(Cc1cccc(N3CCCC3C(F)(F)F)n1)[C@H](CC(C)(C)C)CO2] | | | +++ |
| 550 | 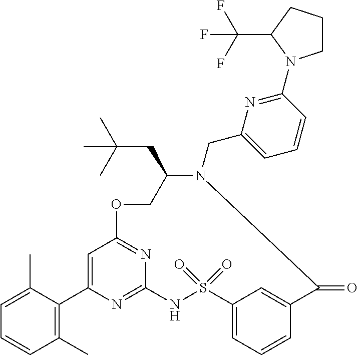 [SMILES: Cc1cccc(C)c1-c1cc2nc(n1)NS(=O)(=O)c1cccc(c1)C(=O)N(Cc1cccc(N3CCCC3C(F)(F)F)n1)[C@H](CC(C)(C)C)CO2] | | | +++ |
| 545 | 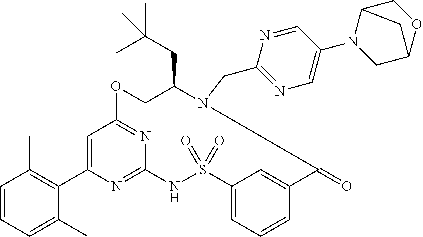 [SMILES: Cc1cccc(C)c1-c1cc2nc(n1)NS(=O)(=O)c1cccc(c1)C(=O)N(Cc1ncc(N3CC4CC3CO4)cn1)[C@H](CC(C)(C)C)CO2] | +++ | +++ | |
| 546 | 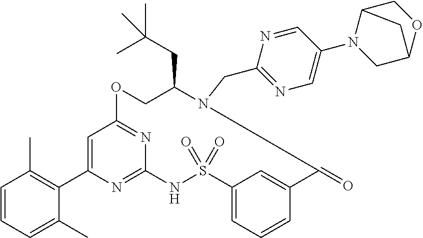 [SMILES: Cc1cccc(C)c1-c1cc2nc(n1)NS(=O)(=O)c1cccc(c1)C(=O)N(Cc1ncc(N3CC4CC3CO4)cn1)[C@H](CC(C)(C)C)CO2] | +++ | +++ | |
| 544 | 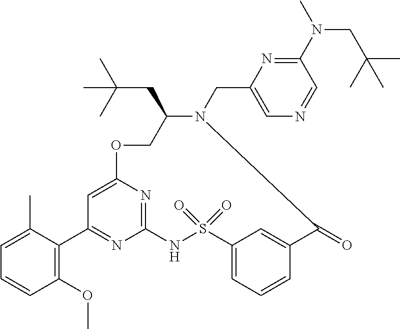 [SMILES: COc1cccc(C)c1-c1cc2nc(n1)NS(=O)(=O)c1cccc(c1)C(=O)N(Cc1cncc(N(C)CC(C)(C)C)n1)[C@H](CC(C)(C)C)CO2] | | | +++ |
| 278 | 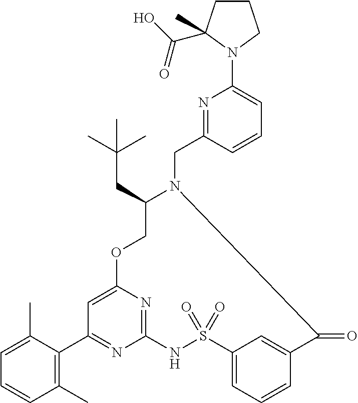 [SMILES: Cc1cccc(C)c1-c1cc2nc(n1)NS(=O)(=O)c1cccc(c1)C(=O)N(Cc1cccc(N3CCC[C@@]3(C)C(=O)O)n1)[C@H](CC(C)(C)C)CO2] | | | +++ |
| 277 | 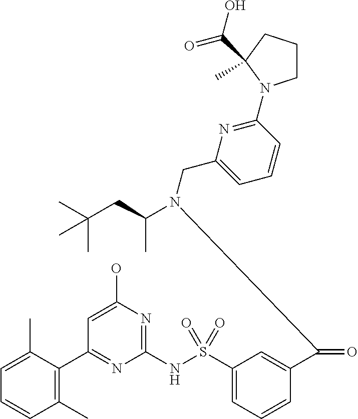 [SMILES: Cc1cccc(C)c1-c1cc(O)nc(NS(=O)(=O)c2cccc(C(=O)N(Cc3cccc(N4CCC[C@]4(C)C(=O)O)n3)[C@@H](C)CC(C)(C)C)c2)n1] | | | +++ |
| 276 | 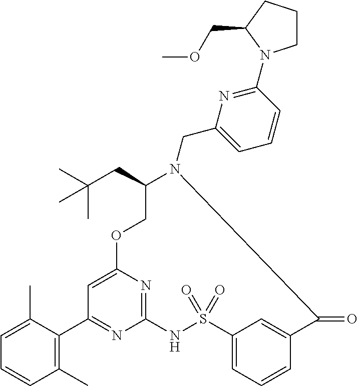 [SMILES: COC[C@H]1CCCN1c1cccc(CN2C(=O)c3cccc(c3)S(=O)(=O)Nc3nc(cc(-c4c(C)cccc4C)n3)OC[C@H]2CC(C)(C)C)n1] | +++ | +++ | |
| 275 | 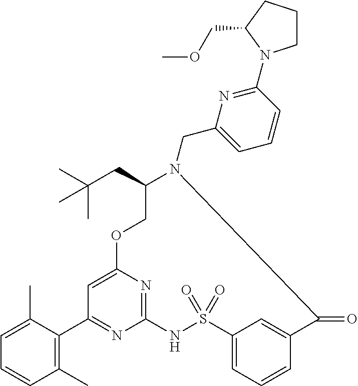 [SMILES: COC[C@@H]1CCCN1c1cccc(CN2C(=O)c3cccc(c3)S(=O)(=O)Nc3nc(cc(-c4c(C)cccc4C)n3)OC[C@H]2CC(C)(C)C)n1] | +++ | +++ | |
| 274 | 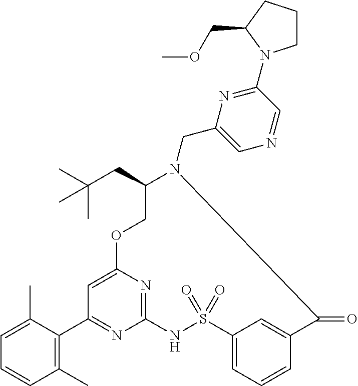 [SMILES: COC[C@H]1CCCN1c1cncc(CN2C(=O)c3cccc(c3)S(=O)(=O)Nc3nc(cc(-c4c(C)cccc4C)n3)OC[C@H]2CC(C)(C)C)n1] | +++ | +++ | |
| 273 | 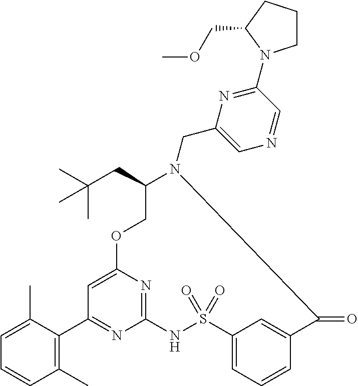 [SMILES: COC[C@@H]1CCCN1c1cncc(CN2C(=O)c3cccc(c3)S(=O)(=O)Nc3nc(cc(-c4c(C)cccc4C)n3)OC[C@H]2CC(C)(C)C)n1] | +++ | +++ | |
| 547 | 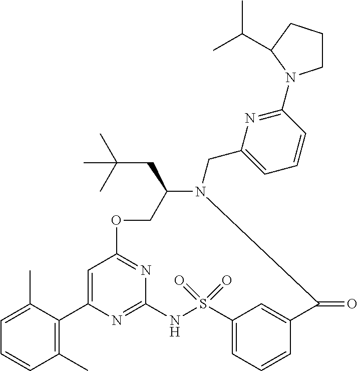 [SMILES: Cc1cccc(C)c1-c1cc2nc(n1)NS(=O)(=O)c1cccc(c1)C(=O)N(Cc1cccc(N3CCCC3C(C)C)n1)[C@H](CC(C)(C)C)CO2] | +++ | +++ | |
| 548 | 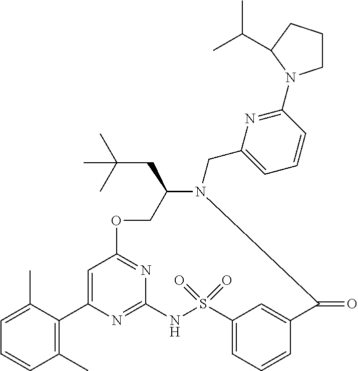 [SMILES: Cc1cccc(C)c1-c1cc2nc(n1)NS(=O)(=O)c1cccc(c1)C(=O)N(Cc1cccc(N3CCCC3C(C)C)n1)[C@H](CC(C)(C)C)CO2] | +++ | +++ | |
| 551 | 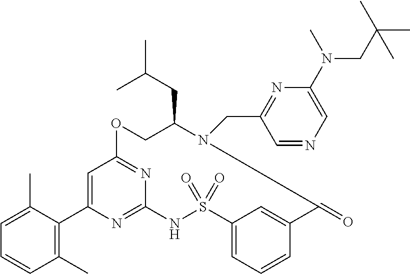 [SMILES: Cc1cccc(C)c1-c1cc2nc(n1)NS(=O)(=O)c1cccc(c1)C(=O)N(Cc1cncc(N(C)CC(C)(C)C)n1)[C@H](CC(C)C)CO2] | +++ | +++ | |
| 552 | 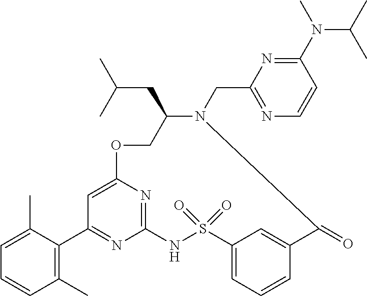 [SMILES: Cc1cccc(C)c1-c1cc2nc(n1)NS(=O)(=O)c1cccc(c1)C(=O)N(Cc1nccc(N(C)C(C)C)n1)[C@H](CC(C)C)CO2] | | | +++ |
| 543 | 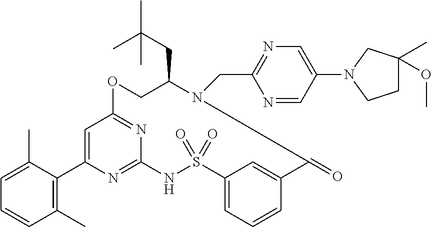 [SMILES: COC1(C)CCN(c2cnc(CN3C(=O)c4cccc(c4)S(=O)(=O)Nc4nc(cc(-c5c(C)cccc5C)n4)OC[C@H]3CC(C)(C)C)nc2)C1] | +++ | +++ | |
| 542 | 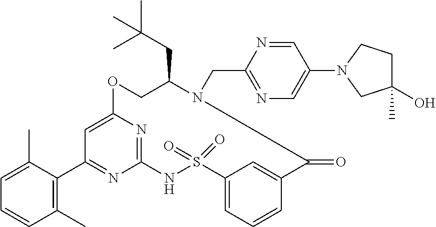 [SMILES: Cc1cccc(C)c1-c1cc2nc(n1)NS(=O)(=O)c1cccc(c1)C(=O)N(Cc1ncc(N3CC[C@@](C)(O)C3)cn1)[C@H](CC(C)(C)C)CO2] | +++ | +++ | |
| 541 | 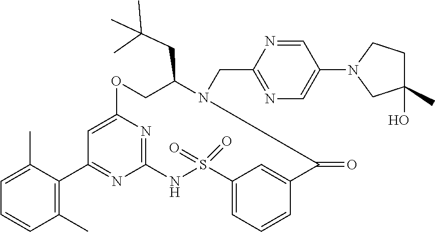 [SMILES: Cc1cccc(C)c1-c1cc2nc(n1)NS(=O)(=O)c1cccc(c1)C(=O)N(Cc1ncc(N3CC[C@](C)(O)C3)cn1)[C@H](CC(C)(C)C)CO2] | +++ | +++ | |
| 239 | 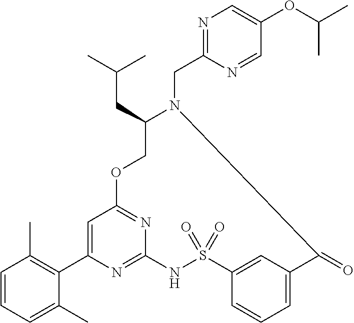 [SMILES: Cc1cccc(C)c1-c1cc2nc(n1)NS(=O)(=O)c1cccc(c1)C(=O)N(Cc1ncc(OC(C)C)cn1)[C@H](CC(C)C)CO2] | ++ | +++ | |
| 693 | 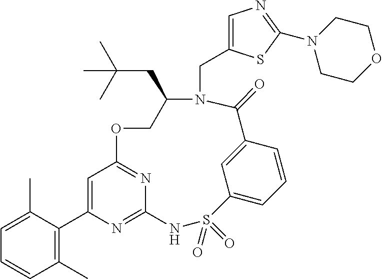 [SMILES: Cc1cccc(C)c1-c1cc2nc(n1)NS(=O)(=O)c1cccc(c1)C(=O)N(Cc1cnc(N3CCOCC3)s1)[C@H](CC(C)(C)C)CO2] | | | ++ |
| 1004 | 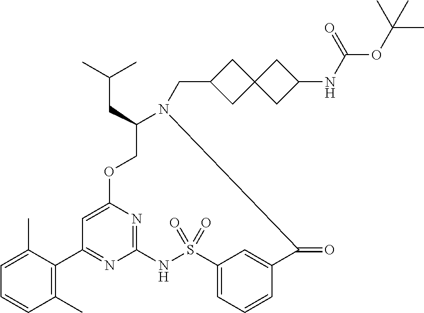 [SMILES: Cc1cccc(C)c1-c1cc2nc(n1)NS(=O)(=O)c1cccc(c1)C(=O)N(CC1CC3(C1)CC(NC(=O)OC(C)(C)C)C3)[C@H](CC(C)C)CO2] | | | +++ |

10. Biological Activity Data

Table 13 represents CFTR modulating activity for representative compounds of the disclosure generated using one or more of the assays disclosed herein (EC₅₀: +++ is <1 μM; ++ is 1-<3 μM; + is 3-<30 μM; and ND is “not detected in this assay.” % Activity: +++ is >600%; ++ is 30-60%; is <30%).

TABLE 13

| | | | HBE | HBE |
|---|---|---|---|---|
| | | HBE | Max | Activity |
| Cmpd | | EC₅₀ | Activity | at 10 μM |
| Number | Structure | (μM) | (%) | (%) |
| 225 | 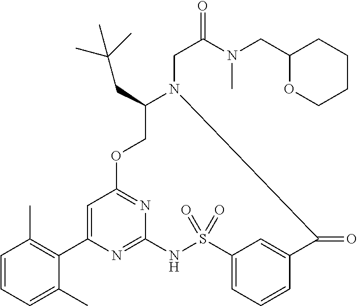 [SMILES: Cc1cccc(C)c1-c1cc2nc(n1)NS(=O)(=O)c1cccc(c1)C(=O)N(CC(=O)N(C)CC1CCCCO1)[C@H](CC(C)(C)C)CO2] | | | +++ |
| 436 | 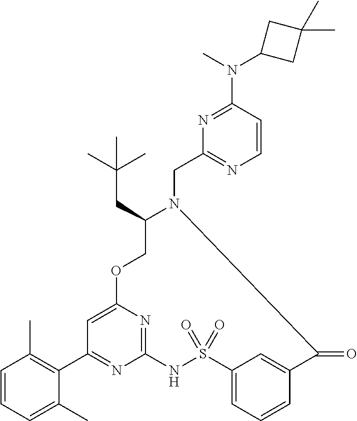 [SMILES: Cc1cccc(C)c1-c1cc2nc(n1)NS(=O)(=O)c1cccc(c1)C(=O)N(Cc1nccc(N(C)C3CC(C)(C)C3)n1)[C@H](CC(C)(C)C)CO2] | +++ | +++ | |
| 435 | 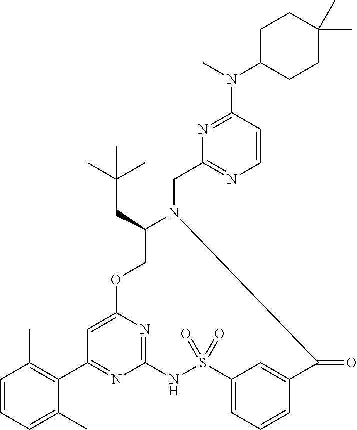 [SMILES: Cc1cccc(C)c1-c1cc2nc(n1)NS(=O)(=O)c1cccc(c1)C(=O)N(Cc1nccc(N(C)C3CCC(C)(C)CC3)n1)[C@H](CC(C)(C)C)CO2] | +++ | +++ | |
| 226 | 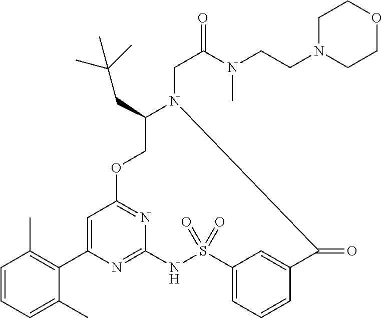 [SMILES: Cc1cccc(C)c1-c1cc2nc(n1)NS(=O)(=O)c1cccc(c1)C(=O)N(CC(=O)N(C)CCN1CCOCC1)[C@H](CC(C)(C)C)CO2] | | | +++ |
| 206 | 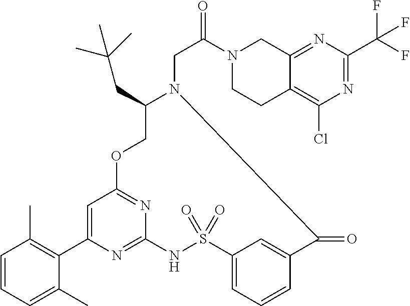 [SMILES: Cc1cccc(C)c1-c1cc2nc(n1)NS(=O)(=O)c1cccc(c1)C(=O)N(CC(=O)N1CCc3c(Cl)nc(C(F)(F)F)nc3C1)[C@H](CC(C)(C)C)CO2] | | | ++ |
| 207 | 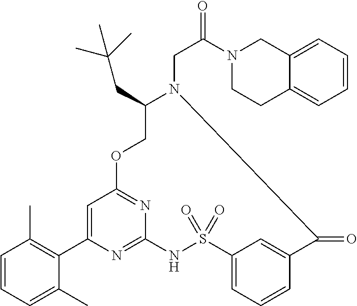 [SMILES: Cc1cccc(C)c1-c1cc2nc(n1)NS(=O)(=O)c1cccc(c1)C(=O)N(CC(=O)N1CCc3ccccc3C1)[C@H](CC(C)(C)C)CO2] | | | +++ |
| 208 | 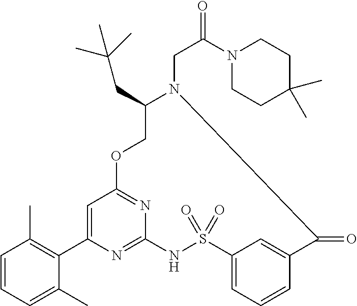 [SMILES: Cc1cccc(C)c1-c1cc2nc(n1)NS(=O)(=O)c1cccc(c1)C(=O)N(CC(=O)N1CCC(C)(C)CC1)[C@H](CC(C)(C)C)CO2] | | | +++ |
| 205 | 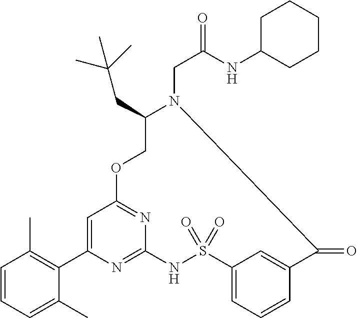 [SMILES: Cc1cccc(C)c1-c1cc2nc(n1)NS(=O)(=O)c1cccc(c1)C(=O)N(CC(=O)NC1CCCCC1)[C@H](CC(C)(C)C)CO2] | | | +++ |
| 201 | 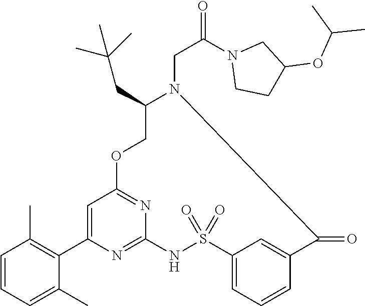 [SMILES: Cc1cccc(C)c1-c1cc2nc(n1)NS(=O)(=O)c1cccc(c1)C(=O)N(CC(=O)N1CCC(OC(C)C)C1)[C@H](CC(C)(C)C)CO2] | | | +++ |
| 200 | 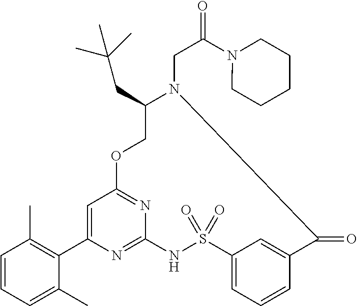 [SMILES: Cc1cccc(C)c1-c1cc2nc(n1)NS(=O)(=O)c1cccc(c1)C(=O)N(CC(=O)N1CCCCC1)[C@H](CC(C)(C)C)CO2] | | | +++ |
| 199 | 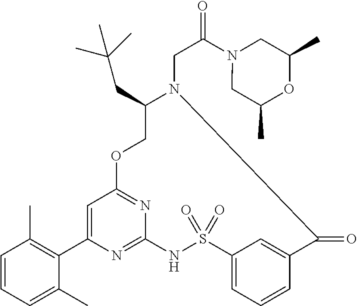 [SMILES: Cc1cccc(C)c1-c1cc2nc(n1)NS(=O)(=O)c1cccc(c1)C(=O)N(CC(=O)N1C[C@@H](C)O[C@@H](C)C1)[C@H](CC(C)(C)C)CO2] | | | +++ |
| 198 | 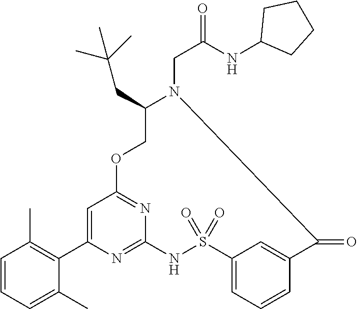 [SMILES: Cc1cccc(C)c1-c1cc2nc(n1)NS(=O)(=O)c1cccc(c1)C(=O)N(CC(=O)NC1CCCC1)[C@H](CC(C)(C)C)CO2] | | | +++ |
| 197 | 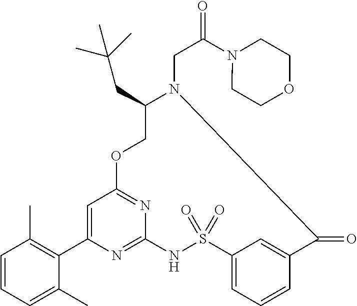 [SMILES: Cc1cccc(C)c1-c1cc2nc(n1)NS(=O)(=O)c1cccc(c1)C(=O)N(CC(=O)N1CCOCC1)[C@H](CC(C)(C)C)CO2] | | | +++ |
| 196 | 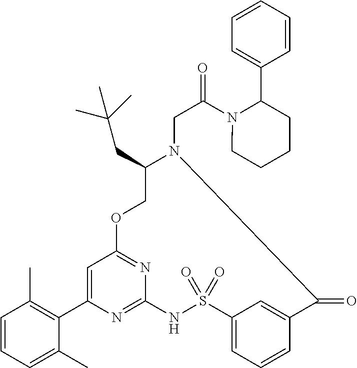 [SMILES: Cc1cccc(C)c1-c1cc2nc(n1)NS(=O)(=O)c1cccc(c1)C(=O)N(CC(=O)N1CCCCC1c1ccccc1)[C@H](CC(C)(C)C)CO2] | | | +++ |
| 195 | 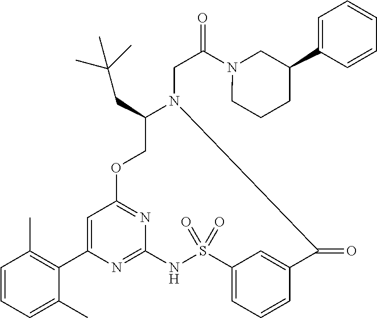 [SMILES: Cc1cccc(C)c1-c1cc2nc(n1)NS(=O)(=O)c1cccc(c1)C(=O)N(CC(=O)N1CCC[C@H](c3ccccc3)C1)[C@H](CC(C)(C)C)CO2] | | | +++ |
| 204 | 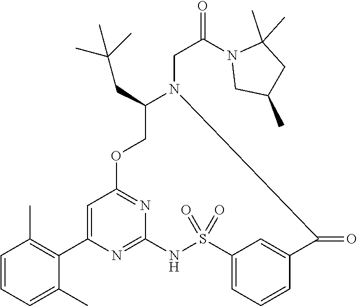 [SMILES: Cc1cccc(C)c1-c1cc2nc(n1)NS(=O)(=O)c1cccc(c1)C(=O)N(CC(=O)N1C[C@H](C)CC1(C)C)[C@H](CC(C)(C)C)CO2] | | | +++ |
| 203 | 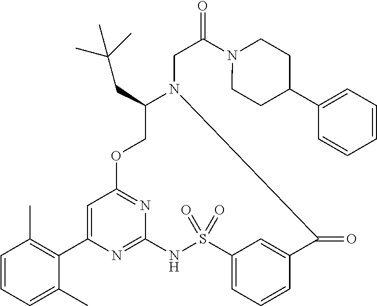 [SMILES: Cc1cccc(C)c1-c1cc2nc(n1)NS(=O)(=O)c1cccc(c1)C(=O)N(CC(=O)N1CCC(c3ccccc3)CC1)[C@H](CC(C)(C)C)CO2] | | | +++ |
| 202 | 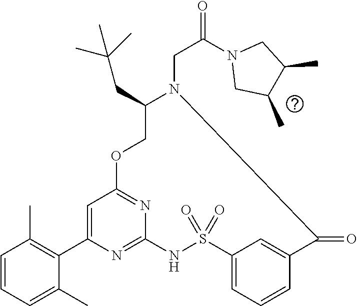 [SMILES: Cc1cccc(C)c1-c1cc2nc(n1)NS(=O)(=O)c1cccc(c1)C(=O)N(CC(=O)N1C[C@@H](C)[C@@H](C)C1)[C@H](CC(C)(C)C)CO2] | | | +++ |
| 223 | 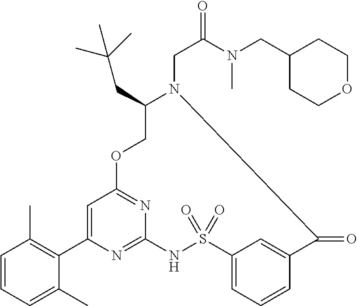 [SMILES: Cc1cccc(C)c1-c1cc2nc(n1)NS(=O)(=O)c1cccc(c1)C(=O)N(CC(=O)N(C)CC1CCOCC1)[C@H](CC(C)(C)C)CO2] | | | +++ |
| 227 | 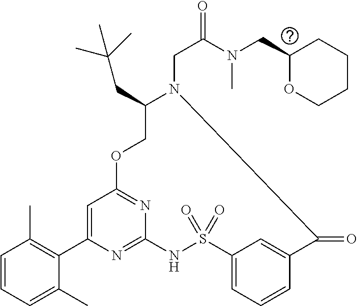 [SMILES: Cc1cccc(C)c1-c1cc2nc(n1)NS(=O)(=O)c1cccc(c1)C(=O)N(CC(=O)N(C)C[C@H]1CCCCO1)[C@H](CC(C)(C)C)CO2] | | | |
| 520 | 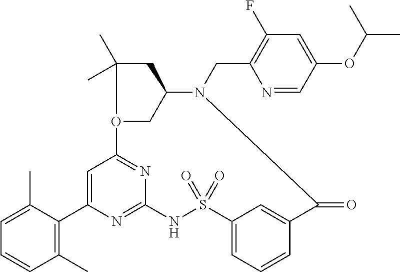 | +++ | +++ | +++ |
| 209 | 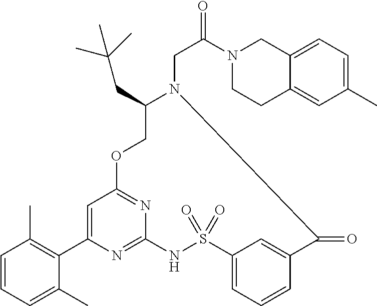 [SMILES: Cc1ccc2c(c1)CCN(C(=O)CN1C(=O)c3cccc(c3)S(=O)(=O)Nc3nc(cc(-c4c(C)cccc4C)n3)OC[C@H]1CC(C)(C)C)C2] | | | +++ |
| 210 | 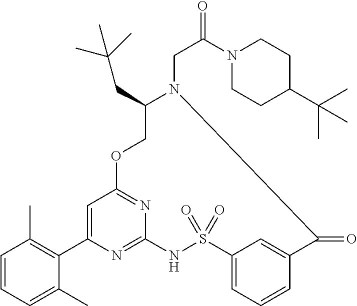 [SMILES: Cc1cccc(C)c1-c1cc2nc(n1)NS(=O)(=O)c1cccc(c1)C(=O)N(CC(=O)N1CCC(C(C)(C)C)CC1)[C@H](CC(C)(C)C)CO2] | | | +++ |
| 1063 | 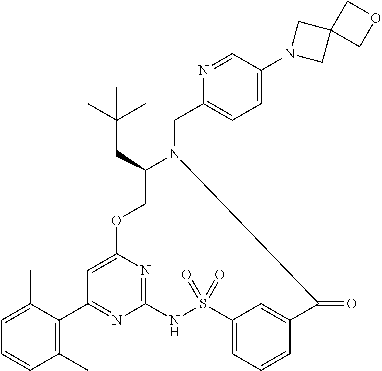 [SMILES: Cc1cccc(C)c1-c1cc2nc(n1)NS(=O)(=O)c1cccc(c1)C(=O)N(Cc1ccc(N3CC4(COC4)C3)cn1)[C@H](CC(C)(C)C)CO2] | +++ | +++ | |
| 539 | 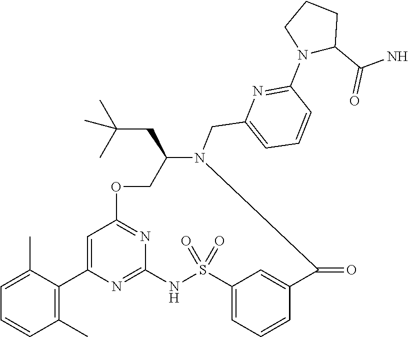 [SMILES: Cc1cccc(C)c1-c1cc2nc(n1)NS(=O)(=O)c1cccc(c1)C(=O)N(Cc1cccc(N3CCCC3C(N)=O)n1)[C@H](CC(C)(C)C)CO2] | | | +++ |
| 540 | 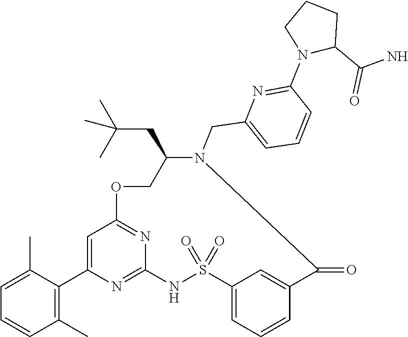 [SMILES: Cc1cccc(C)c1-c1cc2nc(n1)NS(=O)(=O)c1cccc(c1)C(=O)N(Cc1cccc(N3CCCC3C(N)=O)n1)[C@H](CC(C)(C)C)CO2] | | | +++ |
| 538 | 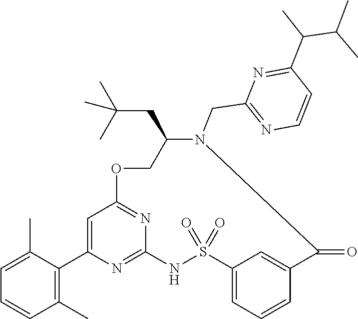 [SMILES: Cc1cccc(C)c1-c1cc2nc(n1)NS(=O)(=O)c1cccc(c1)C(=O)N(Cc1nccc(C(C)C(C)C)n1)[C@H](CC(C)(C)C)CO2] | | | +++ |
| 537 | 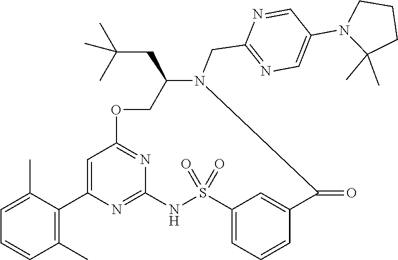 [SMILES: Cc1cccc(C)c1-c1cc2nc(n1)NS(=O)(=O)c1cccc(c1)C(=O)N(Cc1ncc(N3CCCC3(C)C)cn1)[C@H](CC(C)(C)C)CO2] | +++ | +++ | |
| 433 | 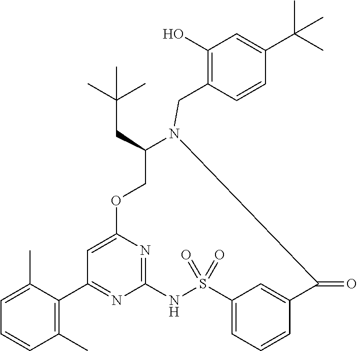 [SMILES: Cc1cccc(C)c1-c1cc2nc(n1)NS(=O)(=O)c1cccc(c1)C(=O)N(Cc1ccc(C(C)(C)C)cc1O)[C@H](CC(C)(C)C)CO2] | | | +++ |
| 434 | 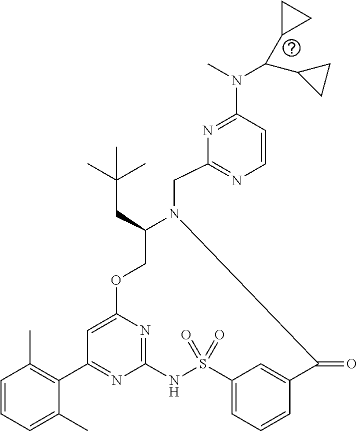 [SMILES: Cc1cccc(C)c1-c1cc2nc(n1)NS(=O)(=O)c1cccc(c1)C(=O)N(Cc1nccc(N(C)C(C3CC3)C3CC3)n1)[C@H](CC(C)(C)C)CO2] | | | +++ |
| 194 | 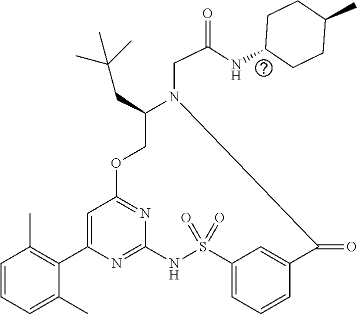 [SMILES: Cc1cccc(C)c1-c1cc2nc(n1)NS(=O)(=O)c1cccc(c1)C(=O)N(CC(=O)N[C@H]1CC[C@H](C)CC1)[C@H](CC(C)(C)C)CO2] | | | +++ |
| 190 | 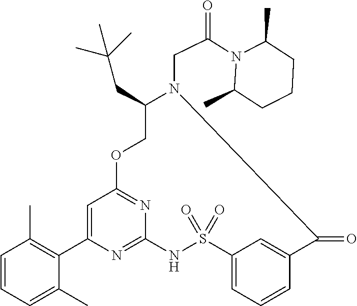 [SMILES: Cc1cccc(C)c1-c1cc2nc(n1)NS(=O)(=O)c1cccc(c1)C(=O)N(CC(=O)N1[C@H](C)CCC[C@@H]1C)[C@H](CC(C)(C)C)CO2] | | | +++ |
| 189 | 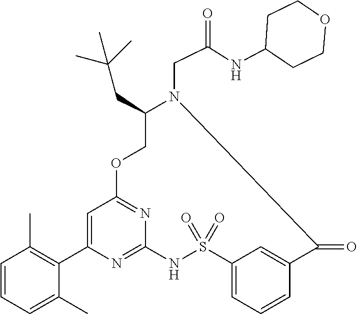 [SMILES: Cc1cccc(C)c1-c1cc2nc(n1)NS(=O)(=O)c1cccc(c1)C(=O)N(CC(=O)NC1CCOCC1)[C@H](CC(C)(C)C)CO2] | | | +++ |
| 157 | 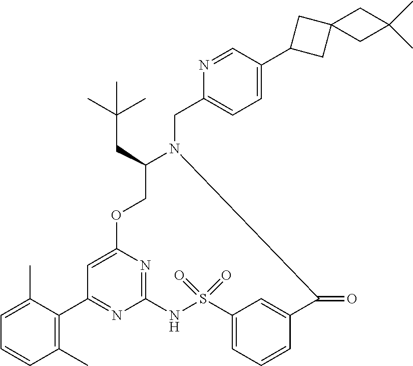 [SMILES: Cc1cccc(C)c1-c1cc2nc(n1)NS(=O)(=O)c1cccc(c1)C(=O)N(Cc1ccc(C3CC4(C3)CC(C)(C)C4)cn1)[C@H](CC(C)(C)C)CO2] | +++ | +++ | |
| 164 | 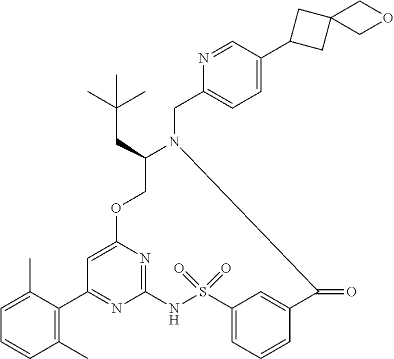 [SMILES: Cc1cccc(C)c1-c1cc2nc(n1)NS(=O)(=O)c1cccc(c1)C(=O)N(Cc1ccc(C3CC4(COC4)C3)cn1)[C@H](CC(C)(C)C)CO2] | +++ | +++ | |
| 188 | 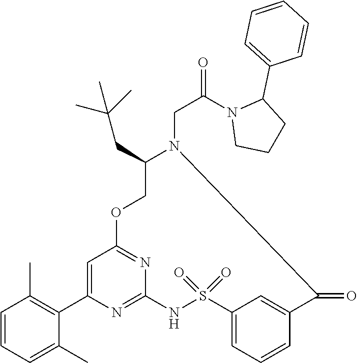 [SMILES: Cc1cccc(C)c1-c1cc2nc(n1)NS(=O)(=O)c1cccc(c1)C(=O)N(CC(=O)N1CCCC1c1ccccc1)[C@H](CC(C)(C)C)CO2] | | | +++ |
| 187 | 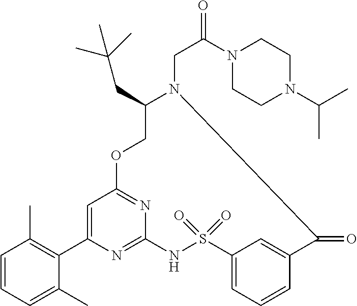 [SMILES: Cc1cccc(C)c1-c1cc2nc(n1)NS(=O)(=O)c1cccc(c1)C(=O)N(CC(=O)N1CCN(C(C)C)CC1)[C@H](CC(C)(C)C)CO2] | | | +++ |
| 186 | 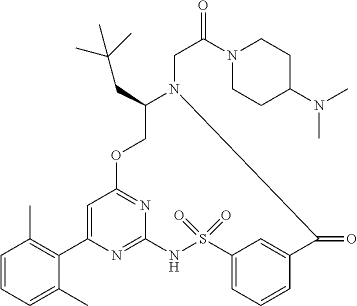 [SMILES: Cc1cccc(C)c1-c1cc2nc(n1)NS(=O)(=O)c1cccc(c1)C(=O)N(CC(=O)N1CCC(N(C)C)CC1)[C@H](CC(C)(C)C)CO2] | | | +++ |
| 185 | 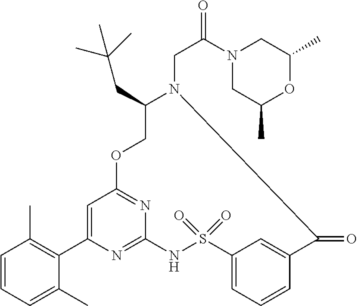 [SMILES: Cc1cccc(C)c1-c1cc2nc(n1)NS(=O)(=O)c1cccc(c1)C(=O)N(CC(=O)N1C[C@H](C)O[C@@H](C)C1)[C@H](CC(C)(C)C)CO2] | | | +++ |
| 184 | 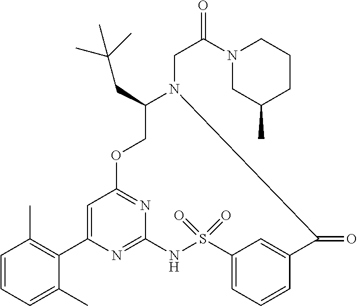 [SMILES: Cc1cccc(C)c1-c1cc2nc(n1)NS(=O)(=O)c1cccc(c1)C(=O)N(CC(=O)N1CCC[C@@H](C)C1)[C@H](CC(C)(C)C)CO2] | | | +++ |
| 193 | 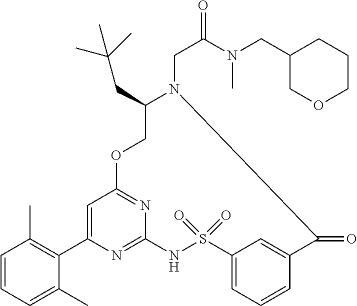 [SMILES: Cc1cccc(C)c1-c1cc2nc(n1)NS(=O)(=O)c1cccc(c1)C(=O)N(CC(=O)N(C)CC1CCCOC1)[C@H](CC(C)(C)C)CO2] | | | +++ |
| 192 | 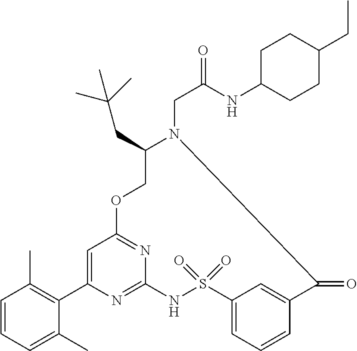 [SMILES: CCC1CCC(NC(=O)CN2C(=O)c3cccc(c3)S(=O)(=O)Nc3nc(cc(-c4c(C)cccc4C)n3)OC[C@H]2CC(C)(C)C)CC1] | | | +++ |
| 191 | 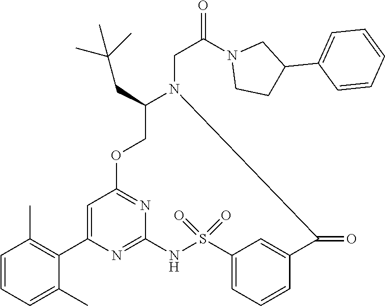 [SMILES: Cc1cccc(C)c1-c1cc2nc(n1)NS(=O)(=O)c1cccc(c1)C(=O)N(CC(=O)N1CCC(c3ccccc3)C1)[C@H](CC(C)(C)C)CO2] | | | +++ |
| 183 | 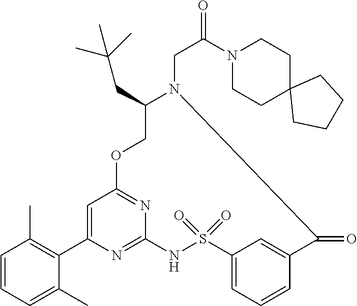 [SMILES: Cc1cccc(C)c1-c1cc2nc(n1)NS(=O)(=O)c1cccc(c1)C(=O)N(CC(=O)N1CCC3(CCCC3)CC1)[C@H](CC(C)(C)C)CO2] | | | +++ |
| 181 | 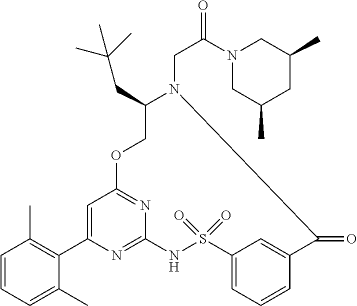 [SMILES: Cc1cccc(C)c1-c1cc2nc(n1)NS(=O)(=O)c1cccc(c1)C(=O)N(CC(=O)N1C[C@H](C)C[C@H](C)C1)[C@H](CC(C)(C)C)CO2] | | | +++ |
| 180 | 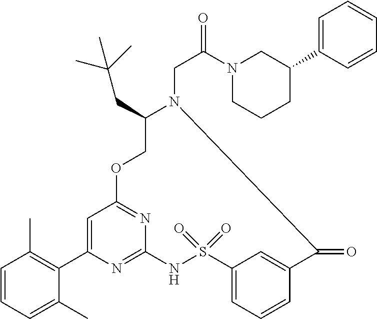 [SMILES: Cc1cccc(C)c1-c1cc2nc(n1)NS(=O)(=O)c1cccc(c1)C(=O)N(CC(=O)N1CCC[C@@H](c3ccccc3)C1)[C@H](CC(C)(C)C)CO2] | | | +++ |
| 179 | 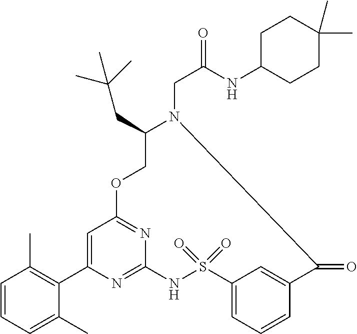 [SMILES: Cc1cccc(C)c1-c1cc2nc(n1)NS(=O)(=O)c1cccc(c1)C(=O)N(CC(=O)NC1CCC(C)(C)CC1)[C@H](CC(C)(C)C)CO2] | | | +++ |
| 178 | 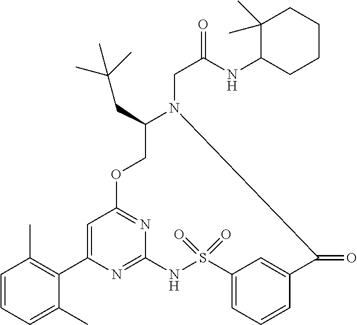 [SMILES: Cc1cccc(C)c1-c1cc2nc(n1)NS(=O)(=O)c1cccc(c1)C(=O)N(CC(=O)NC1CCCCC1(C)C)[C@H](CC(C)(C)C)CO2] | | | +++ |
| 177 | 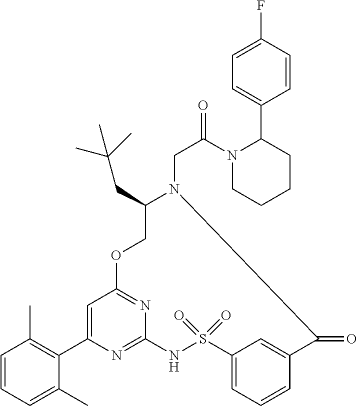 [SMILES: Cc1cccc(C)c1-c1cc2nc(n1)NS(=O)(=O)c1cccc(c1)C(=O)N(CC(=O)N1CCCCC1c1ccc(F)cc1)[C@H](CC(C)(C)C)CO2] | | | +++ |
| 176 | 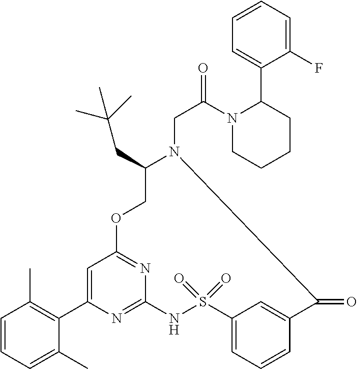 [SMILES: Cc1cccc(C)c1-c1cc2nc(n1)NS(=O)(=O)c1cccc(c1)C(=O)N(CC(=O)N1CCCCC1c1ccccc1F)[C@H](CC(C)(C)C)CO2] | | | +++ |
| 175 | 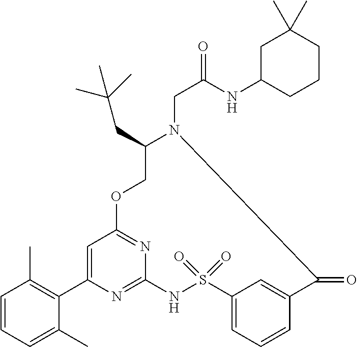 [SMILES: Cc1cccc(C)c1-c1cc2nc(n1)NS(=O)(=O)c1cccc(c1)C(=O)N(CC(=O)NC1CCCC(C)(C)C1)[C@H](CC(C)(C)C)CO2] | | | +++ |
| 182 | 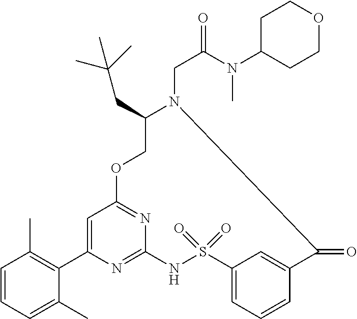 [SMILES: Cc1cccc(C)c1-c1cc2nc(n1)NS(=O)(=O)c1cccc(c1)C(=O)N(CC(=O)N(C)C1CCOCC1)[C@H](CC(C)(C)C)CO2] | | | +++ |
| 219 | 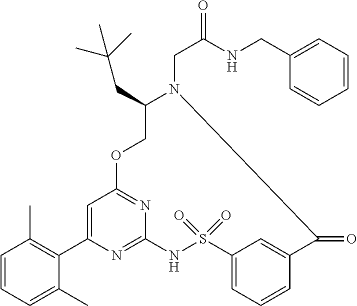 [SMILES: Cc1cccc(C)c1-c1cc2nc(n1)NS(=O)(=O)c1cccc(c1)C(=O)N(CC(=O)NCc1ccccc1)[C@H](CC(C)(C)C)CO2] | | | +++ |
| 220 | 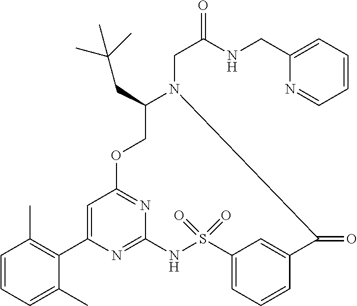 [SMILES: Cc1cccc(C)c1-c1cc2nc(n1)NS(=O)(=O)c1cccc(c1)C(=O)N(CC(=O)NCc1ccccn1)[C@H](CC(C)(C)C)CO2] | | | +++ |
| 211 | 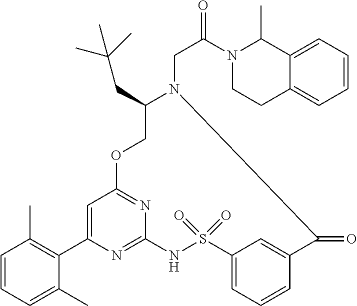 [SMILES: Cc1cccc(C)c1-c1cc2nc(n1)NS(=O)(=O)c1cccc(c1)C(=O)N(CC(=O)N1CCc3ccccc3C1C)[C@H](CC(C)(C)C)CO2] | | | +++ |
| 519 | 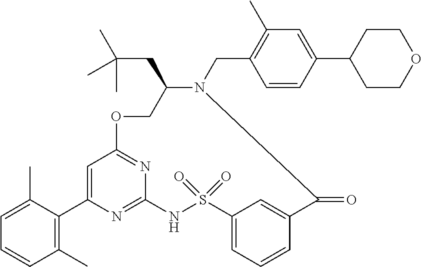 [SMILES: Cc1cc(C2CCOCC2)ccc1CN1C(=O)c2cccc(c2)S(=O)(=O)Nc2nc(cc(-c3c(C)cccc3C)n2)OC[C@H]1CC(C)(C)C] | | | +++ |
| 518 | 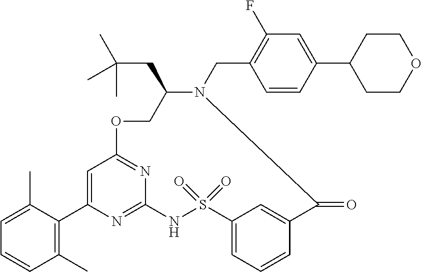 [SMILES: Cc1cccc(C)c1-c1cc2nc(n1)NS(=O)(=O)c1cccc(c1)C(=O)N(Cc1ccc(C3CCOCC3)cc1F)[C@H](CC(C)(C)C)CO2] | | | +++ |
| 517 | 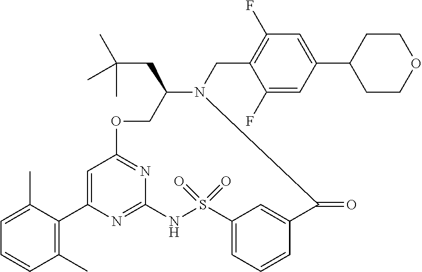 [SMILES: Cc1cccc(C)c1-c1cc2nc(n1)NS(=O)(=O)c1cccc(c1)C(=O)N(Cc1c(F)cc(C3CCOCC3)cc1F)[C@H](CC(C)(C)C)CO2] | | | +++ |
| 212 | 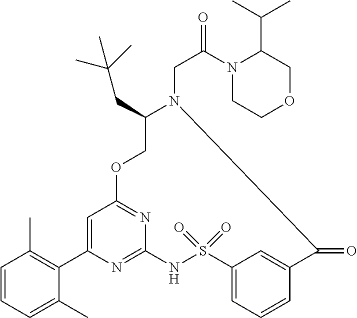 [SMILES: Cc1cccc(C)c1-c1cc2nc(n1)NS(=O)(=O)c1cccc(c1)C(=O)N(CC(=O)N1CCOCC1C(C)C)[C@H](CC(C)(C)C)CO2] | | | +++ |
| 213 | 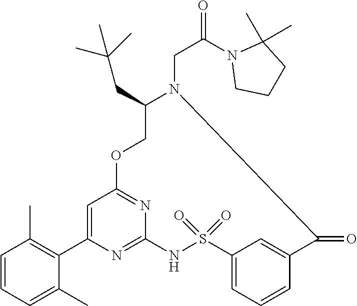 [SMILES: Cc1cccc(C)c1-c1cc2nc(n1)NS(=O)(=O)c1cccc(c1)C(=O)N(CC(=O)N1CCCC1(C)C)[C@H](CC(C)(C)C)CO2] | | | +++ |
| 214 | 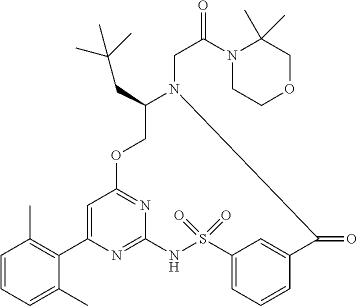 [SMILES: Cc1cccc(C)c1-c1cc2nc(n1)NS(=O)(=O)c1cccc(c1)C(=O)N(CC(=O)N1CCOCC1(C)C)[C@H](CC(C)(C)C)CO2] | | | +++ |
| 259 | 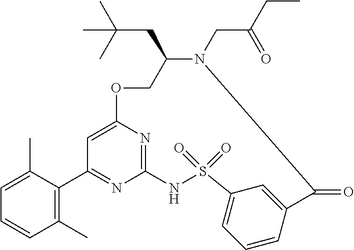 [SMILES: CCC(=O)CN1C(=O)c2cccc(c2)S(=O)(=O)Nc2nc(cc(-c3c(C)cccc3C)n2)OC[C@H]1CC(C)(C)C] | | | +++ |
| 258 | 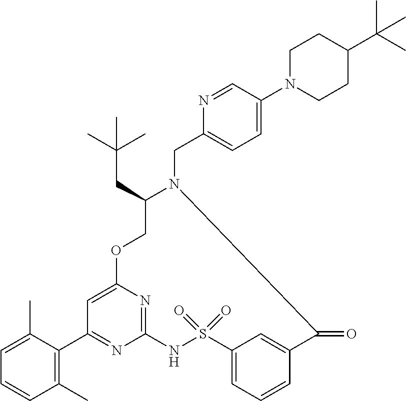 [SMILES: Cc1cccc(C)c1-c1cc2nc(n1)NS(=O)(=O)c1cccc(c1)C(=O)N(Cc1ccc(N3CCC(C(C)(C)C)CC3)cn1)[C@H](CC(C)(C)C)CO2] | | | +++ |
| 257 | 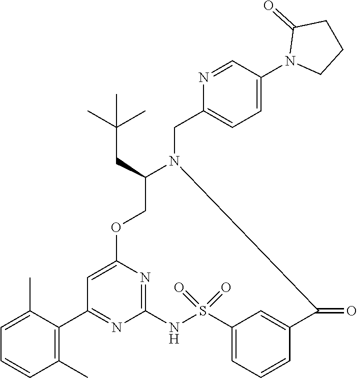 [SMILES: Cc1cccc(C)c1-c1cc2nc(n1)NS(=O)(=O)c1cccc(c1)C(=O)N(Cc1ccc(N3CCCC3=O)cn1)[C@H](CC(C)(C)C)CO2] | | | +++ |
| 1000 | 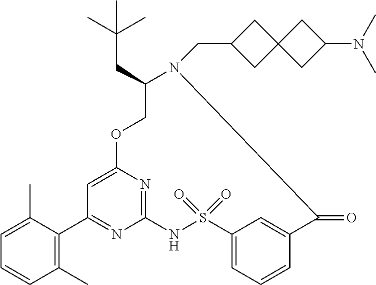 [SMILES: Cc1cccc(C)c1-c1cc2nc(n1)NS(=O)(=O)c1cccc(c1)C(=O)N(CC1CC3(C1)CC(N(C)C)C3)[C@H](CC(C)(C)C)CO2] | | | +++ |
| 1003 | 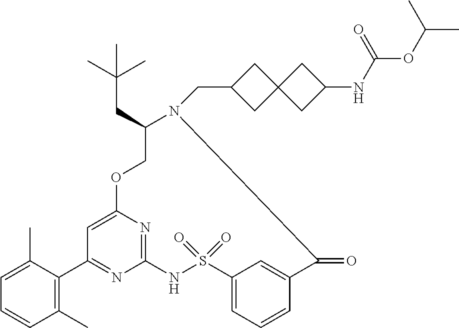 [SMILES: Cc1cccc(C)c1-c1cc2nc(n1)NS(=O)(=O)c1cccc(c1)C(=O)N(CC1CC3(C1)CC(NC(=O)OC(C)C)C3)[C@H](CC(C)(C)C)CO2] | | | +++ |
| 1002 | 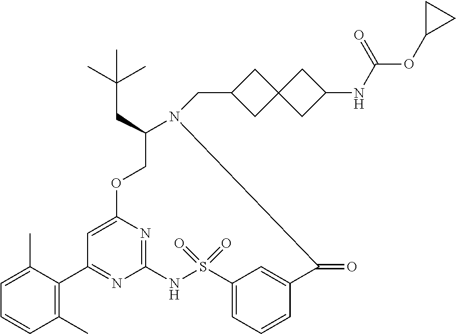 [SMILES: Cc1cccc(C)c1-c1cc2nc(n1)NS(=O)(=O)c1cccc(c1)C(=O)N(CC1CC3(C1)CC(NC(=O)OC1CC1)C3)[C@H](CC(C)(C)C)CO2] | | | +++ |
| 1001 | 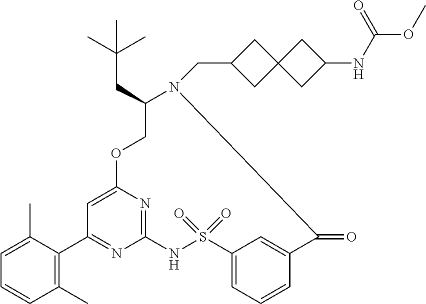 [SMILES: COC(=O)NC1CC2(CC(CN3C(=O)c4cccc(c4)S(=O)(=O)Nc4nc(cc(-c5c(C)cccc5C)n4)OC[C@H]3CC(C)(C)C)C2)C1] | | | +++ |
| 256 | 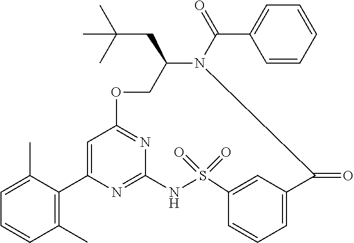 [SMILES: Cc1cccc(C)c1-c1cc2nc(n1)NS(=O)(=O)c1cccc(c1)C(=O)N(C(=O)c1ccccc1)[C@H](CC(C)(C)C)CO2] | | | +++ |
| 255 | 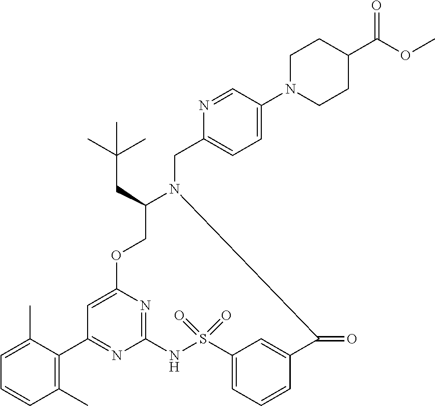 [SMILES: COC(=O)C1CCN(c2ccc(CN3C(=O)c4cccc(c4)S(=O)(=O)Nc4nc(cc(-c5c(C)cccc5C)n4)OC[C@H]3CC(C)(C)C)nc2)CC1] | | | +++ |
| 254 | 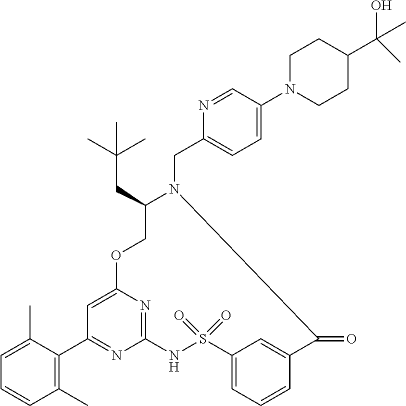 [SMILES: Cc1cccc(C)c1-c1cc2nc(n1)NS(=O)(=O)c1cccc(c1)C(=O)N(Cc1ccc(N3CCC(C(C)(C)O)CC3)cn1)[C@H](CC(C)(C)C)CO2] | | | +++ |
| 1057 | 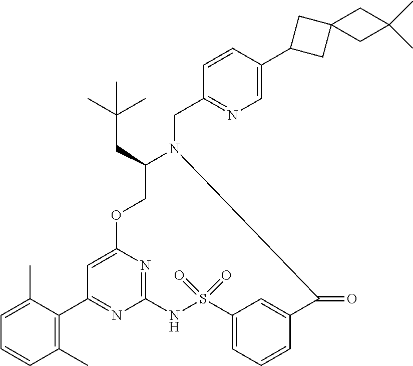 [SMILES: Cc1cccc(C)c1-c1cc2nc(n1)NS(=O)(=O)c1cccc(c1)C(=O)N(Cc1ccc(C3CC4(C3)CC(C)(C)C4)cn1)[C@H](CC(C)(C)C)CO2] | | | +++ |
| 1062 | 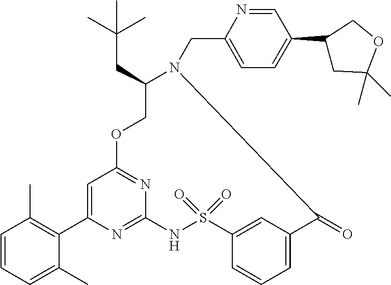 | | | +++ |
| 1061 | 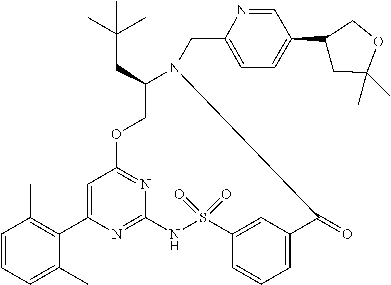 | | | +++ |
| 253 | 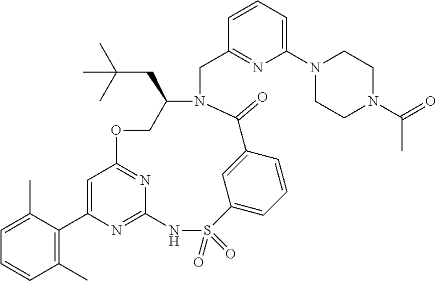 [SMILES: CC(=O)N1CCN(c2cccc(CN3C(=O)c4cccc(c4)S(=O)(=O)Nc4nc(cc(-c5c(C)cccc5C)n4)OC[C@H]3CC(C)(C)C)n2)CC1] | | | +++ |
| 536 | 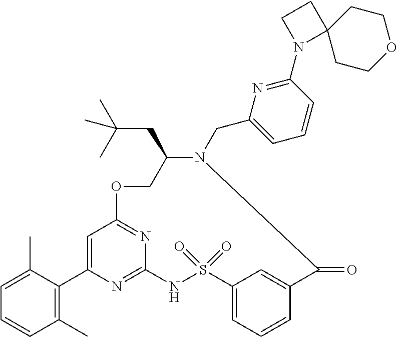 [SMILES: Cc1cccc(C)c1-c1cc2nc(n1)NS(=O)(=O)c1cccc(c1)C(=O)N(Cc1cccc(N3CCC34CCOCC4)n1)[C@H](CC(C)(C)C)CO2] | +++ | +++ | +++ |
| 535 | 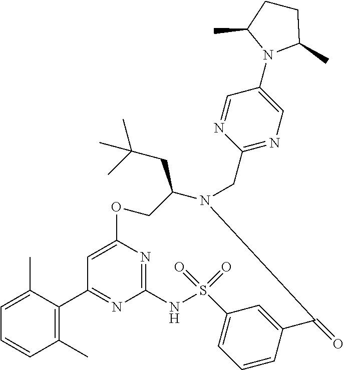 [SMILES: Cc1cccc(C)c1-c1cc2nc(n1)NS(=O)(=O)c1cccc(c1)C(=O)N(Cc1ncc(N3[C@H](C)CC[C@@H]3C)cn1)[C@H](CC(C)(C)C)CO2] | +++ | +++ | +++ |
| 173 | 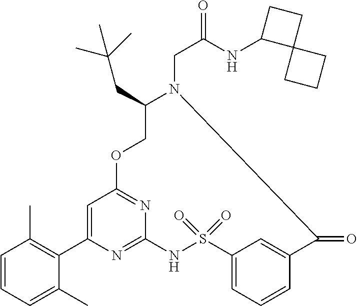 [SMILES: Cc1cccc(C)c1-c1cc2nc(n1)NS(=O)(=O)c1cccc(c1)C(=O)N(CC(=O)NC1CCC13CCC3)[C@H](CC(C)(C)C)CO2] | | | +++ |
| 172 | 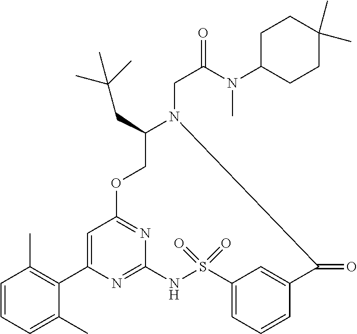 [SMILES: Cc1cccc(C)c1-c1cc2nc(n1)NS(=O)(=O)c1cccc(c1)C(=O)N(CC(=O)N(C)C1CCC(C)(C)CC1)[C@H](CC(C)(C)C)CO2] | | | +++ |
| 171 | 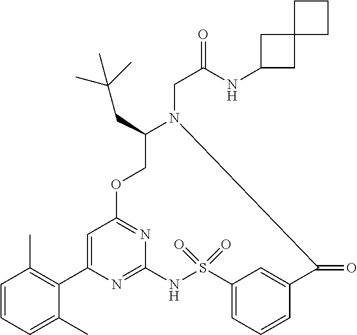 [SMILES: Cc1cccc(C)c1-c1cc2nc(n1)NS(=O)(=O)c1cccc(c1)C(=O)N(CC(=O)NC1CC3(CCC3)C1)[C@H](CC(C)(C)C)CO2] | | | +++ |
| 170 | 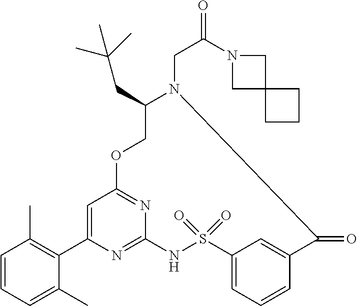 [SMILES: Cc1cccc(C)c1-c1cc2nc(n1)NS(=O)(=O)c1cccc(c1)C(=O)N(CC(=O)N1CC3(CCC3)C1)[C@H](CC(C)(C)C)CO2] | | | ++ |
| 169 | 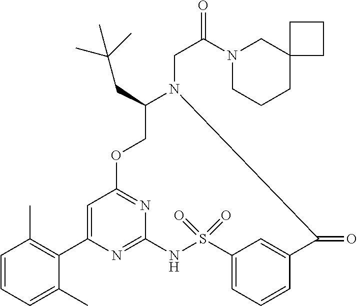 [SMILES: Cc1cccc(C)c1-c1cc2nc(n1)NS(=O)(=O)c1cccc(c1)C(=O)N(CC(=O)N1CCCC3(CCC3)C1)[C@H](CC(C)(C)C)CO2] | | | +++ |
| 168 | 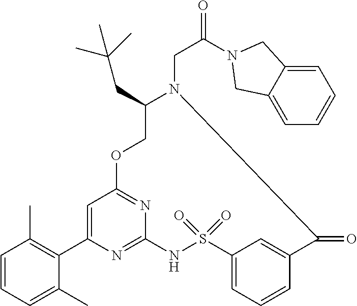 [SMILES: Cc1cccc(C)c1-c1cc2nc(n1)NS(=O)(=O)c1cccc(c1)C(=O)N(CC(=O)N1Cc3ccccc3C1)[C@H](CC(C)(C)C)CO2] | | | +++ |
| 870 | 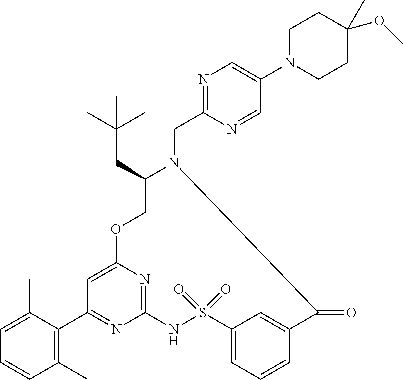 [SMILES: COC1(C)CCN(c2cnc(CN3C(=O)c4cccc(c4)S(=O)(=O)Nc4nc(cc(-c5c(C)cccc5C)n4)OC[C@H]3CC(C)(C)C)nc2)CC1] | +++ | +++ | +++ |
| 999 | 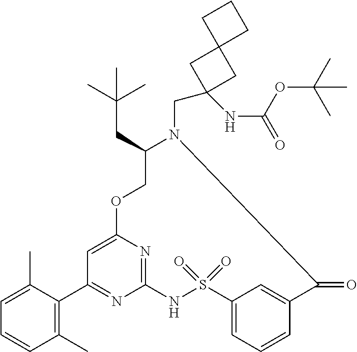 [SMILES: Cc1cccc(C)c1-c1cc2nc(n1)NS(=O)(=O)c1cccc(c1)C(=O)N(CC1(NC(=O)OC(C)(C)C)CC3(CCC3)C1)[C@H](CC(C)(C)C)CO2] | | | +++ |
| 252 | 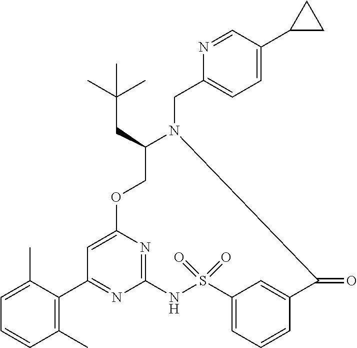 [SMILES: Cc1cccc(C)c1-c1cc2nc(n1)NS(=O)(=O)c1cccc(c1)C(=O)N(Cc1ccc(C3CC3)cn1)[C@H](CC(C)(C)C)CO2] | +++ | +++ | |
| 1060 | 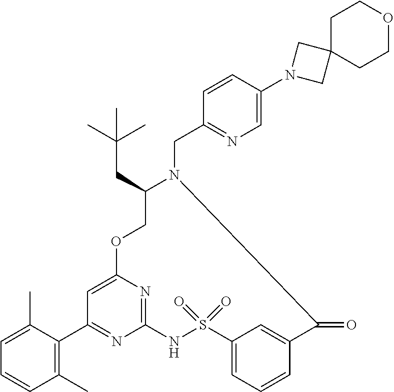 [SMILES: Cc1cccc(C)c1-c1cc2nc(n1)NS(=O)(=O)c1cccc(c1)C(=O)N(Cc1ccc(N3CC4(CCOCC4)C3)cn1)[C@H](CC(C)(C)C)CO2] | +++ | +++ | |
| 163 | 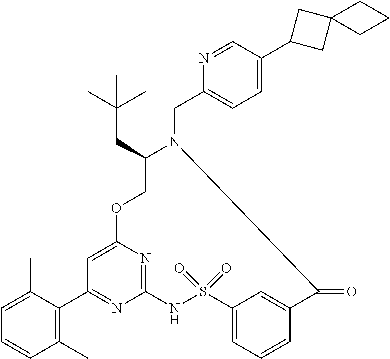 [SMILES: Cc1cccc(C)c1-c1cc2nc(n1)NS(=O)(=O)c1cccc(c1)C(=O)N(Cc1ccc(C3CC4(CCC4)C3)cn1)[C@H](CC(C)(C)C)CO2] | ++ | +++ | |
| 1058 | 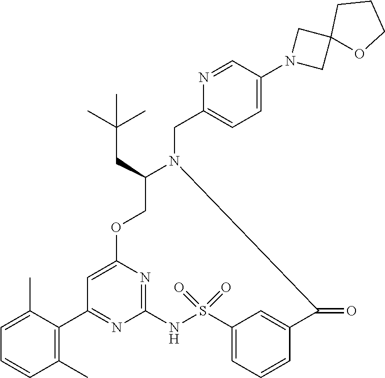 [SMILES: Cc1cccc(C)c1-c1cc2nc(n1)NS(=O)(=O)c1cccc(c1)C(=O)N(Cc1ccc(N3CC4(CCCO4)C3)cn1)[C@H](CC(C)(C)C)CO2] | +++ | +++ | |
| 1059 | 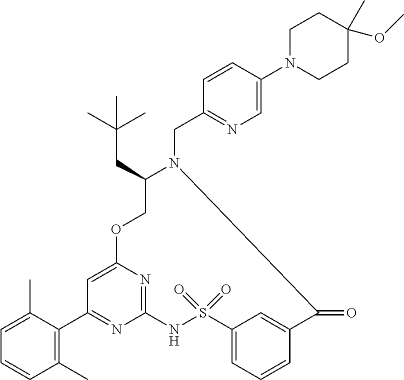 [SMILES: COC1(C)CCN(c2ccc(CN3C(=O)c4cccc(c4)S(=O)(=O)Nc4nc(cc(-c5c(C)cccc5C)n4)OC[C@H]3CC(C)(C)C)nc2)CC1] | +++ | +++ | |
| 251 | 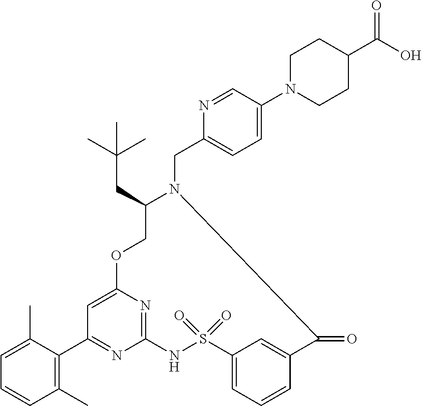 [SMILES: Cc1cccc(C)c1-c1cc2nc(n1)NS(=O)(=O)c1cccc(c1)C(=O)N(Cc1ccc(N3CCC(C(=O)O)CC3)cn1)[C@H](CC(C)(C)C)CO2] | | | ++ |
| 998 | 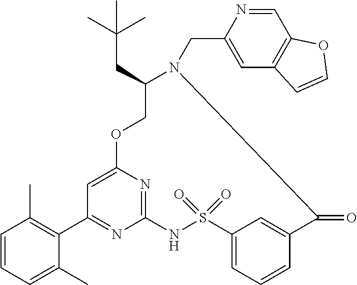 [SMILES: Cc1cccc(C)c1-c1cc2nc(n1)NS(=O)(=O)c1cccc(c1)C(=O)N(Cc1cc3ccoc3cn1)[C@H](CC(C)(C)C)CO2] | | | +++ |
| 249 | 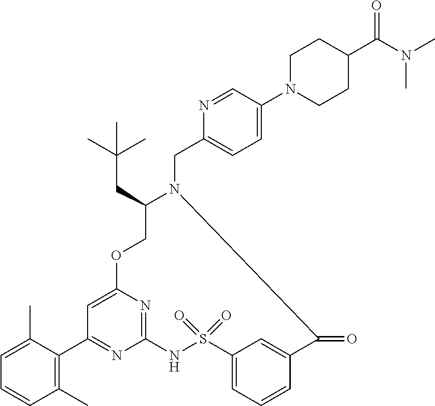 [SMILES: Cc1cccc(C)c1-c1cc2nc(n1)NS(=O)(=O)c1cccc(c1)C(=O)N(Cc1ccc(N3CCC(C(=O)N(C)C)CC3)cn1)[C@H](CC(C)(C)C)CO2] | | | +++ |
| 997 | 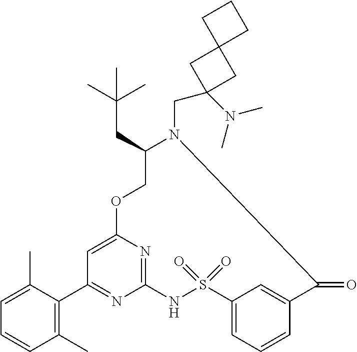 [SMILES: Cc1cccc(C)c1-c1cc2nc(n1)NS(=O)(=O)c1cccc(c1)C(=O)N(CC1(N(C)C)CC3(CCC3)C1)[C@H](CC(C)(C)C)CO2] | | | +++ |
| 248 | 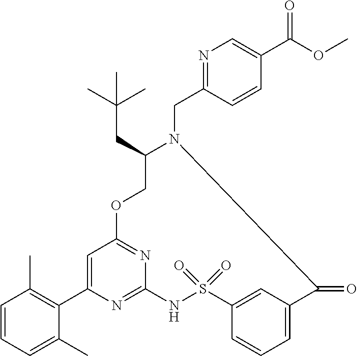 [SMILES: COC(=O)c1ccc(CN2C(=O)c3cccc(c3)S(=O)(=O)Nc3nc(cc(-c4c(C)cccc4C)n3)OC[C@H]2CC(C)(C)C)nc1] | | | +++ |
| 247 | 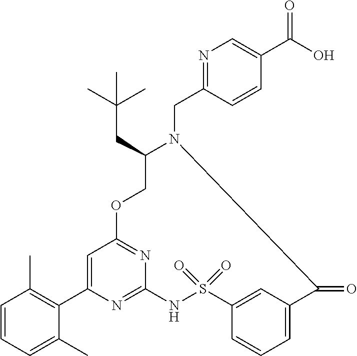 [SMILES: Cc1cccc(C)c1-c1cc2nc(n1)NS(=O)(=O)c1cccc(c1)C(=O)N(Cc1ccc(C(=O)O)cn1)[C@H](CC(C)(C)C)CO2] | | | ++ |
| 533 | 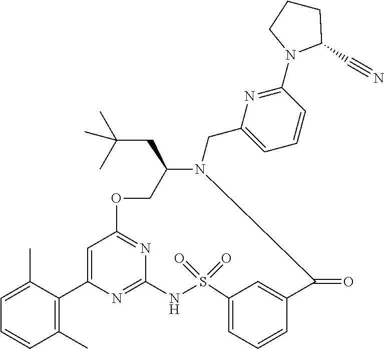 [SMILES: Cc1cccc(C)c1-c1cc2nc(n1)NS(=O)(=O)c1cccc(c1)C(=O)N(Cc1cccc(N3CCC[C@@H]3C#N)n1)[C@H](CC(C)(C)C)CO2] | | | +++ |
| 534 | 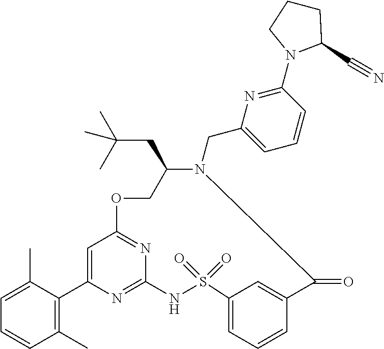 [SMILES: Cc1cccc(C)c1-c1cc2nc(n1)NS(=O)(=O)c1cccc(c1)C(=O)N(Cc1cccc(N3CCC[C@H]3C#N)n1)[C@H](CC(C)(C)C)CO2] | | | +++ |
| 165 | 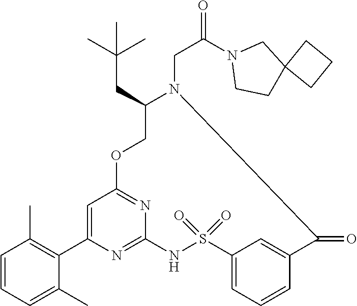 [SMILES: Cc1cccc(C)c1-c1cc2nc(n1)NS(=O)(=O)c1cccc(c1)C(=O)N(CC(=O)N1CCC3(CCC3)C1)[C@H](CC(C)(C)C)CO2] | | | +++ |
| 246 | 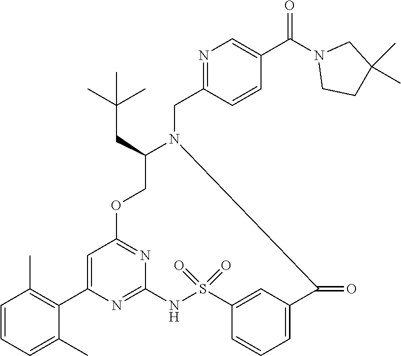 [SMILES: Cc1cccc(C)c1-c1cc2nc(n1)NS(=O)(=O)c1cccc(c1)C(=O)N(Cc1ccc(C(=O)N3CCC(C)(C)C3)cn1)[C@H](CC(C)(C)C)CO2] | | | +++ |
| 245 | 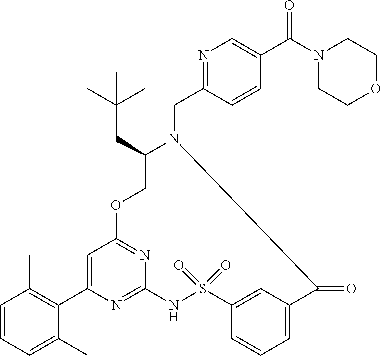 [SMILES: Cc1cccc(C)c1-c1cc2nc(n1)NS(=O)(=O)c1cccc(c1)C(=O)N(Cc1ccc(C(=O)N3CCOCC3)cn1)[C@H](CC(C)(C)C)CO2] | | | +++ |
| 236 | 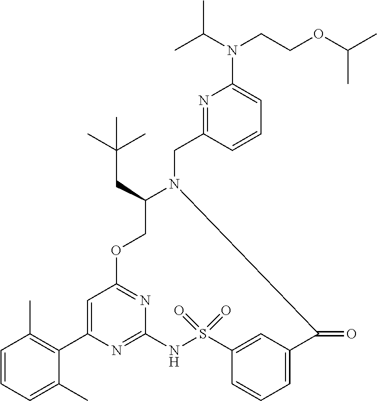 [SMILES: Cc1cccc(C)c1-c1cc2nc(n1)NS(=O)(=O)c1cccc(c1)C(=O)N(Cc1cccc(N(CCOC(C)C)C(C)C)n1)[C@H](CC(C)(C)C)CO2] | ++ | +++ | |
| 532 | 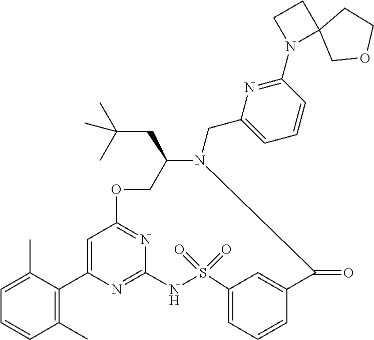 [SMILES: Cc1cccc(C)c1-c1cc2nc(n1)NS(=O)(=O)c1cccc(c1)C(=O)N(Cc1cccc(N3CCC34CCOC4)n1)[C@H](CC(C)(C)C)CO2] | +++ | +++ | |
| 531 | 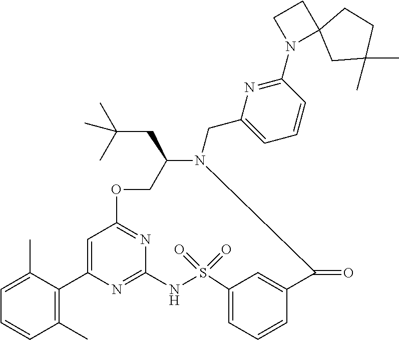 [SMILES: Cc1cccc(C)c1-c1cc2nc(n1)NS(=O)(=O)c1cccc(c1)C(=O)N(Cc1cccc(N3CCC34CCC(C)(C)C4)n1)[C@H](CC(C)(C)C)CO2] | +++ | +++ | |
| 996 | 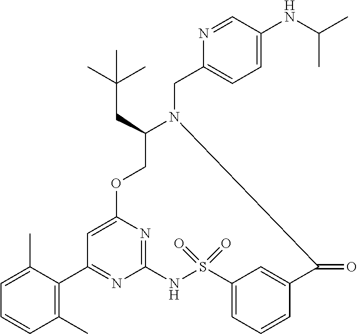 [SMILES: Cc1cccc(C)c1-c1cc2nc(n1)NS(=O)(=O)c1cccc(c1)C(=O)N(Cc1ccc(NC(C)C)cn1)[C@H](CC(C)(C)C)CO2] | +++ | +++ | |
| 162 | 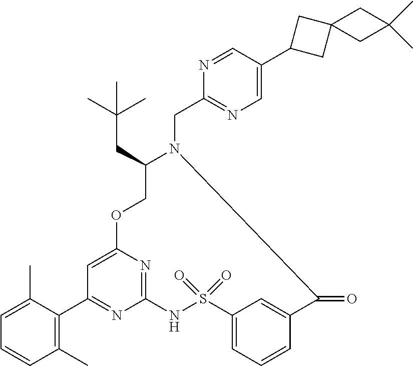 [SMILES: Cc1cccc(C)c1-c1cc2nc(n1)NS(=O)(=O)c1cccc(c1)C(=O)N(Cc1ncc(C3CC4(C3)CC(C)(C)C4)cn1)[C@H](CC(C)(C)C)CO2] | +++ | +++ | |
| 161 | 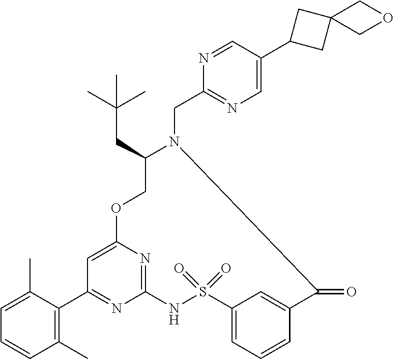 [SMILES: Cc1cccc(C)c1-c1cc2nc(n1)NS(=O)(=O)c1cccc(c1)C(=O)N(Cc1ncc(C3CC4(COC4)C3)cn1)[C@H](CC(C)(C)C)CO2] | +++ | +++ | |
| 160 | 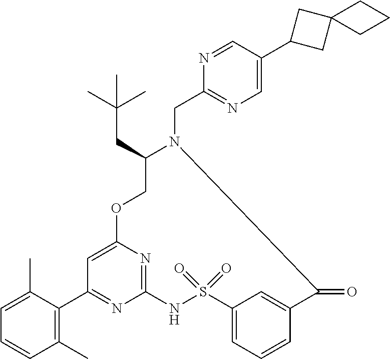 [SMILES: Cc1cccc(C)c1-c1cc2nc(n1)NS(=O)(=O)c1cccc(c1)C(=O)N(Cc1ncc(C3CC4(CCC4)C3)cn1)[C@H](CC(C)(C)C)CO2] | +++ | +++ | |
| 235 | 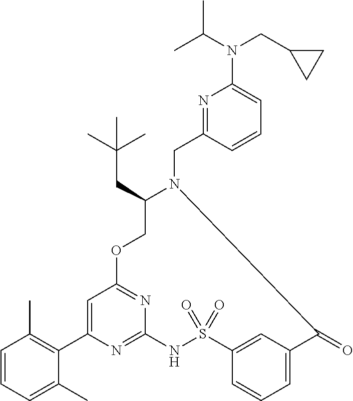 [SMILES: Cc1cccc(C)c1-c1cc2nc(n1)NS(=O)(=O)c1cccc(c1)C(=O)N(Cc1cccc(N(CC3CC3)C(C)C)n1)[C@H](CC(C)(C)C)CO2] | +++ | +++ | |
| 1054 | 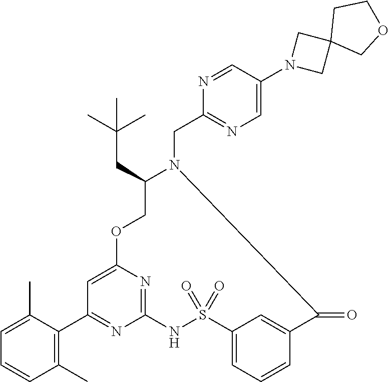 [SMILES: Cc1cccc(C)c1-c1cc2nc(n1)NS(=O)(=O)c1cccc(c1)C(=O)N(Cc1ncc(N3CC4(CCOC4)C3)cn1)[C@H](CC(C)(C)C)CO2] | +++ | +++ | |
| 1055 | 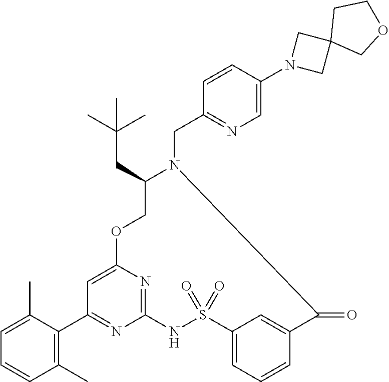 [SMILES: Cc1cccc(C)c1-c1cc2nc(n1)NS(=O)(=O)c1cccc(c1)C(=O)N(Cc1ccc(N3CC4(CCOC4)C3)cn1)[C@H](CC(C)(C)C)CO2] | | | +++ |
| 1056 | 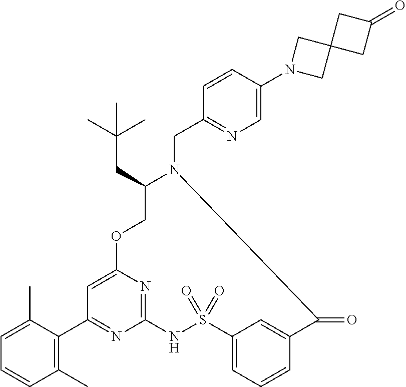 [SMILES: Cc1cccc(C)c1-c1cc2nc(n1)NS(=O)(=O)c1cccc(c1)C(=O)N(Cc1ccc(N3CC4(CC(=O)C4)C3)cn1)[C@H](CC(C)(C)C)CO2] | ++ | +++ | |
| 1053 | 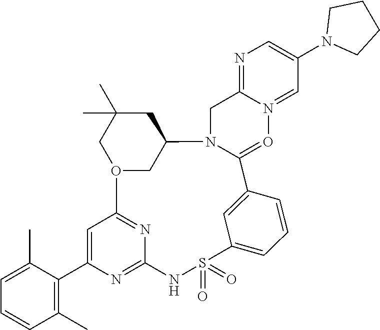 | +++ | +++ | |
| 692 | 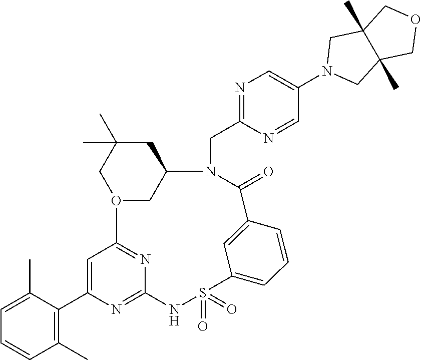 | +++ | +++ | |
| 1068 | 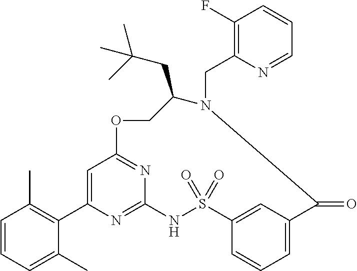 [SMILES: Cc1cccc(C)c1-c1cc2nc(n1)NS(=O)(=O)c1cccc(c1)C(=O)N(Cc1ncccc1F)[C@H](CC(C)(C)C)CO2] | +++ | +++ | |
| 1067 | 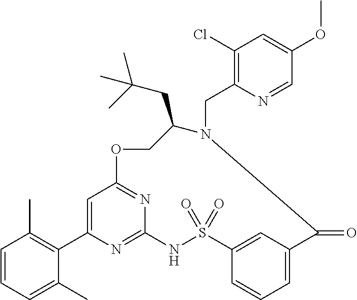 [SMILES: COc1cnc(CN2C(=O)c3cccc(c3)S(=O)(=O)Nc3nc(cc(-c4c(C)cccc4C)n3)OC[C@H]2CC(C)(C)C)c(Cl)c1] | +++ | +++ | |
| 372 | 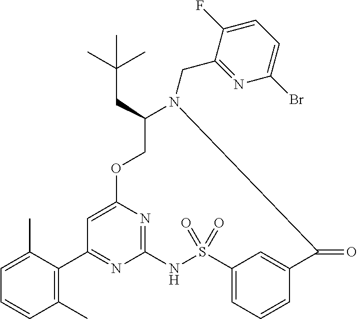 [SMILES: Cc1cccc(C)c1-c1cc2nc(n1)NS(=O)(=O)c1cccc(c1)C(=O)N(Cc1nc(Br)ccc1F)[C@H](CC(C)(C)C)CO2] | | | +++ |
| 1066 | 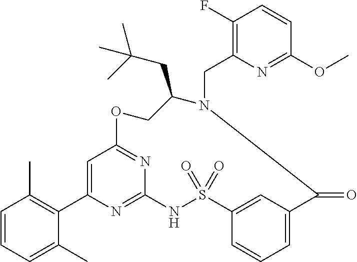 [SMILES: COc1ccc(F)c(CN2C(=O)c3cccc(c3)S(=O)(=O)Nc3nc(cc(-c4c(C)cccc4C)n3)OC[C@H]2CC(C)(C)C)n1] | +++ | +++ | |
| 869 | 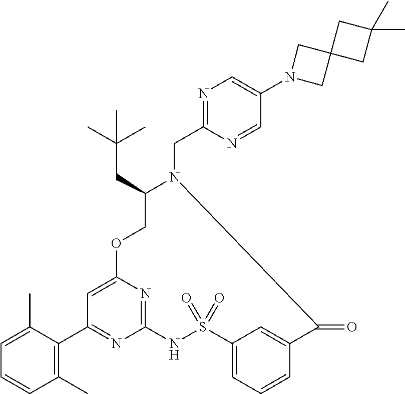 [SMILES: Cc1cccc(C)c1-c1cc2nc(n1)NS(=O)(=O)c1cccc(c1)C(=O)N(Cc1ncc(N3CC4(C3)CC(C)(C)C4)cn1)[C@H](CC(C)(C)C)CO2] | +++ | +++ | |
| 868 | 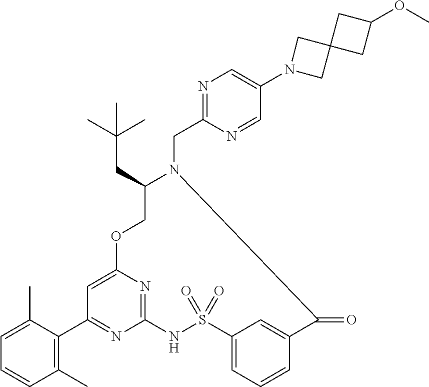 [SMILES: COC1CC2(C1)CN(c1cnc(CN3C(=O)c4cccc(c4)S(=O)(=O)Nc4nc(cc(-c5c(C)cccc5C)n4)OC[C@H]3CC(C)(C)C)nc1)C2] | +++ | +++ | |
| 867 | 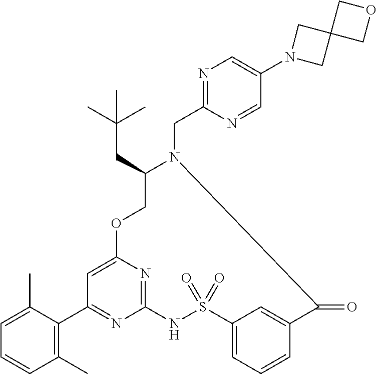 [SMILES: Cc1cccc(C)c1-c1cc2nc(n1)NS(=O)(=O)c1cccc(c1)C(=O)N(Cc1ncc(N3CC4(COC4)C3)cn1)[C@H](CC(C)(C)C)CO2] | +++ | +++ | |
| 866 | 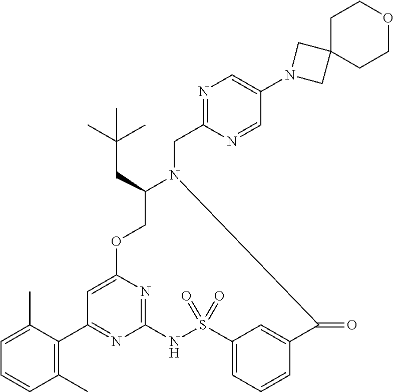 [SMILES: Cc1cccc(C)c1-c1cc2nc(n1)NS(=O)(=O)c1cccc(c1)C(=O)N(Cc1ncc(N3CC4(CCOCC4)C3)cn1)[C@H](CC(C)(C)C)CO2] | +++ | +++ | |
| 530 | 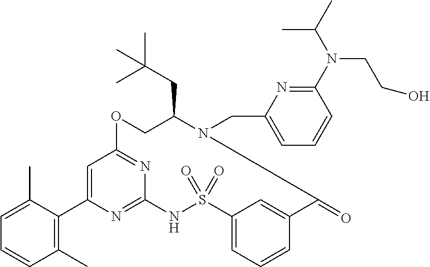 [SMILES: Cc1cccc(C)c1-c1cc2nc(n1)NS(=O)(=O)c1cccc(c1)C(=O)N(Cc1cccc(N(CCO)C(C)C)n1)[C@H](CC(C)(C)C)CO2] | +++ | +++ | |
| 993 | 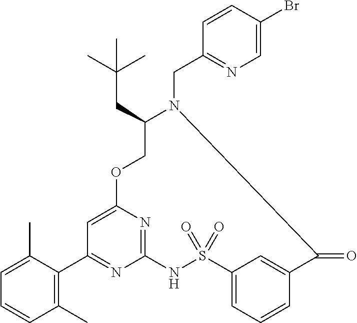 [SMILES: Cc1cccc(C)c1-c1cc2nc(n1)NS(=O)(=O)c1cccc(c1)C(=O)N(Cc1ccc(Br)cn1)[C@H](CC(C)(C)C)CO2] | | | +++ |
| 992 | 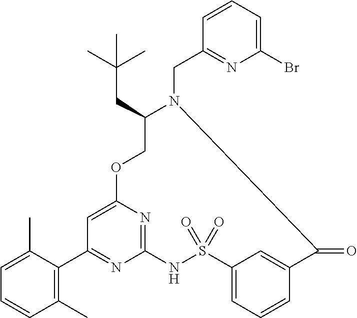 [SMILES: Cc1cccc(C)c1-c1cc2nc(n1)NS(=O)(=O)c1cccc(c1)C(=O)N(Cc1cccc(Br)n1)[C@H](CC(C)(C)C)CO2] | | | +++ |
| 995 | 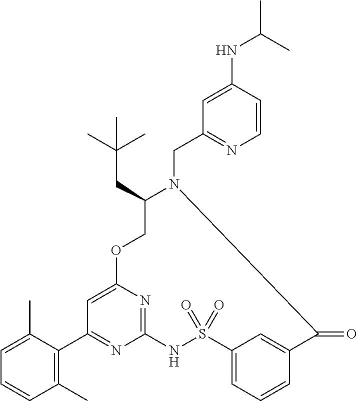 [SMILES: Cc1cccc(C)c1-c1cc2nc(n1)NS(=O)(=O)c1cccc(c1)C(=O)N(Cc1cc(NC(C)C)ccn1)[C@H](CC(C)(C)C)CO2] | | | +++ |
| 994 | 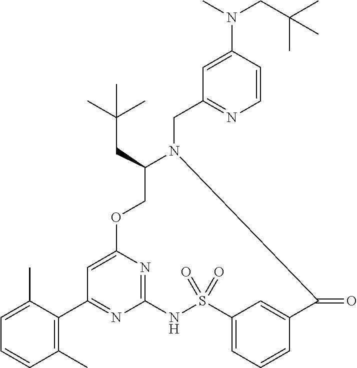 [SMILES: Cc1cccc(C)c1-c1cc2nc(n1)NS(=O)(=O)c1cccc(c1)C(=O)N(Cc1cc(N(C)CC(C)(C)C)ccn1)[C@H](CC(C)(C)C)CO2] | | | +++ |
| 234 | 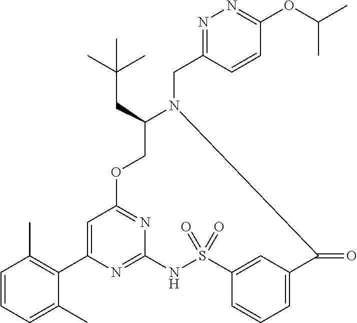 [SMILES: Cc1cccc(C)c1-c1cc2nc(n1)NS(=O)(=O)c1cccc(c1)C(=O)N(Cc1ccc(OC(C)C)nn1)[C@H](CC(C)(C)C)CO2] | +++ | +++ | |
| 244 | 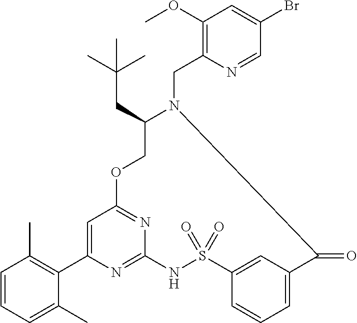 [SMILES: COc1cc(Br)cnc1CN1C(=O)c2cccc(c2)S(=O)(=O)Nc2nc(cc(-c3c(C)cccc3C)n2)OC[C@H]1CC(C)(C)C] | | | +++ |
| 691 | 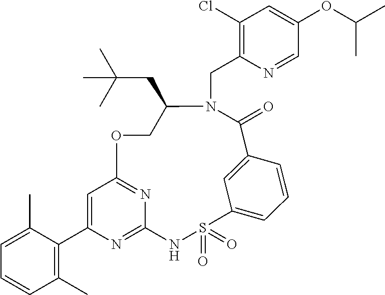 [SMILES: Cc1cccc(C)c1-c1cc2nc(n1)NS(=O)(=O)c1cccc(c1)C(=O)N(Cc1ncc(OC(C)C)cc1Cl)[C@H](CC(C)(C)C)CO2] | +++ | +++ | |
| 690 | 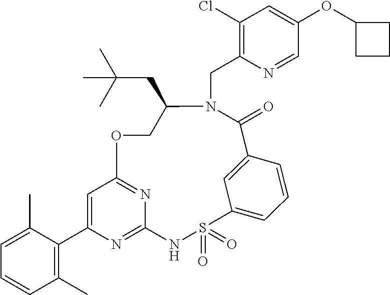 [SMILES: Cc1cccc(C)c1-c1cc2nc(n1)NS(=O)(=O)c1cccc(c1)C(=O)N(Cc1ncc(OC3CCC3)cc1Cl)[C@H](CC(C)(C)C)CO2] | +++ | +++ | |
| 371 | 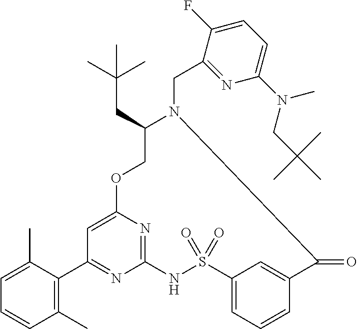 [SMILES: Cc1cccc(C)c1-c1cc2nc(n1)NS(=O)(=O)c1cccc(c1)C(=O)N(Cc1nc(N(C)CC(C)(C)C)ccc1F)[C@H](CC(C)(C)C)CO2] | +++ | +++ | |
| 370 | 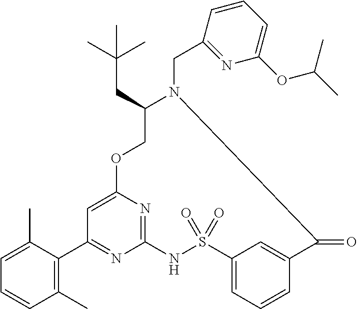 [SMILES: Cc1cccc(C)c1-c1cc2nc(n1)NS(=O)(=O)c1cccc(c1)C(=O)N(Cc1cccc(OC(C)C)n1)[C@H](CC(C)(C)C)CO2] | +++ | +++ | |
| 1115 | 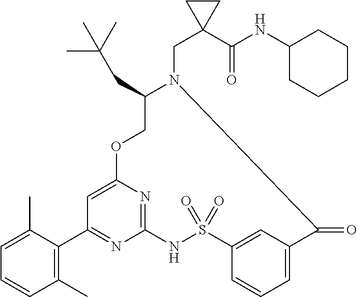 [SMILES: Cc1cccc(C)c1-c1cc2nc(n1)NS(=O)(=O)c1cccc(c1)C(=O)N(CC1(C(=O)NC3CCCCC3)CC1)[C@H](CC(C)(C)C)CO2] | | | +++ |
| 159 | 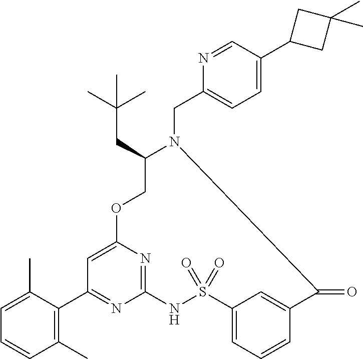 [SMILES: Cc1cccc(C)c1-c1cc2nc(n1)NS(=O)(=O)c1cccc(c1)C(=O)N(Cc1ccc(C3CC(C)(C)C3)cn1)[C@H](CC(C)(C)C)CO2] | +++ | +++ | |
| 1116 | 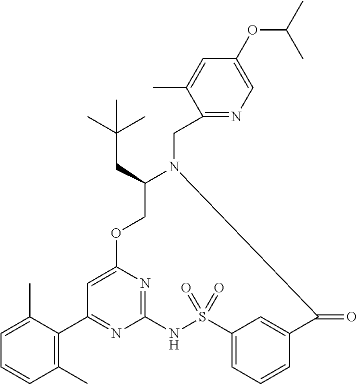 [SMILES: Cc1cc(OC(C)C)cnc1CN1C(=O)c2cccc(c2)S(=O)(=O)Nc2nc(cc(-c3c(C)cccc3C)n2)OC[C@H]1CC(C)(C)C] | +++ | +++ | |
| 158 | 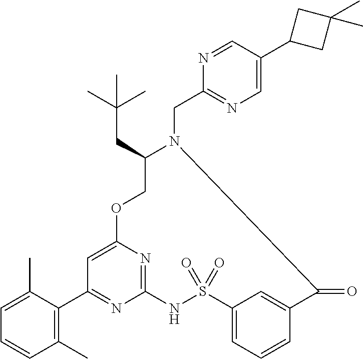 [SMILES: Cc1cccc(C)c1-c1cc2nc(n1)NS(=O)(=O)c1cccc(c1)C(=O)N(Cc1ncc(C3CC(C)(C)C3)cn1)[C@H](CC(C)(C)C)CO2] | +++ | +++ | |
| 1120 | 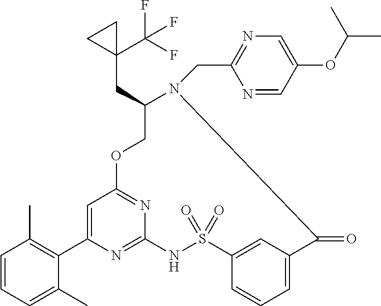 [SMILES: Cc1cccc(C)c1-c1cc2nc(n1)NS(=O)(=O)c1cccc(c1)C(=O)N(Cc1ncc(OC(C)C)cn1)[C@H](CC1(C(F)(F)F)CC1)CO2] | +++ | +++ | |
| 1121 | 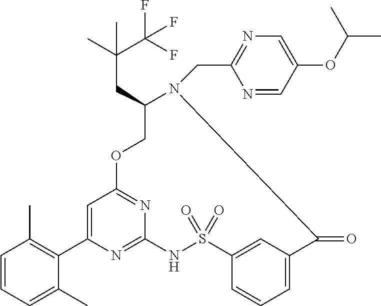 [SMILES: Cc1cccc(C)c1-c1cc2nc(n1)NS(=O)(=O)c1cccc(c1)C(=O)N(Cc1ncc(OC(C)C)cn1)[C@H](CC(C)(C)C(F)(F)F)CO2] | +++ | +++ | |
| 1123 | 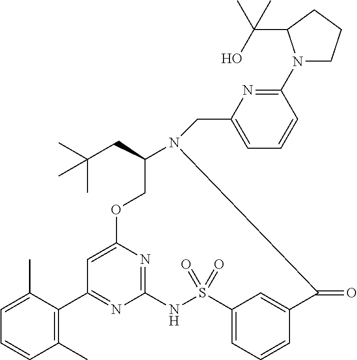 [SMILES: Cc1cccc(C)c1-c1cc2nc(n1)NS(=O)(=O)c1cccc(c1)C(=O)N(Cc1cccc(N3CCCC3C(C)(C)O)n1)[C@H](CC(C)(C)C)CO2] | | | +++ |
| 1122 | 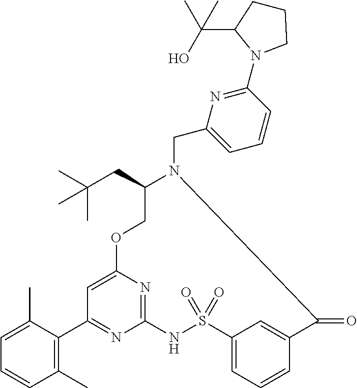 [SMILES: Cc1cccc(C)c1-c1cc2nc(n1)NS(=O)(=O)c1cccc(c1)C(=O)N(Cc1cccc(N3CCCC3C(C)(C)O)n1)[C@H](CC(C)(C)C)CO2] | | | +++ |
| 1118 | 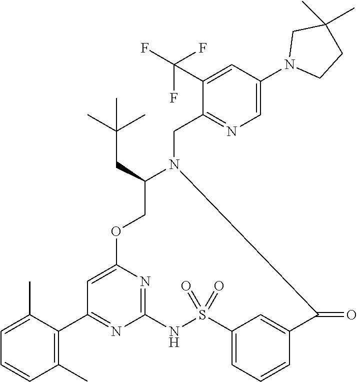 [SMILES: Cc1cccc(C)c1-c1cc2nc(n1)NS(=O)(=O)c1cccc(c1)C(=O)N(Cc1ncc(N3CCC(C)(C)C3)cc1C(F)(F)F)[C@H](CC(C)(C)C)CO2] | +++ | +++ | |
| 1119 | 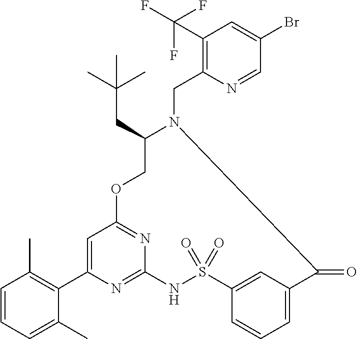 [SMILES: Cc1cccc(C)c1-c1cc2nc(n1)NS(=O)(=O)c1cccc(c1)C(=O)N(Cc1ncc(Br)cc1C(F)(F)F)[C@H](CC(C)(C)C)CO2] | | | +++ |
| 1132 | 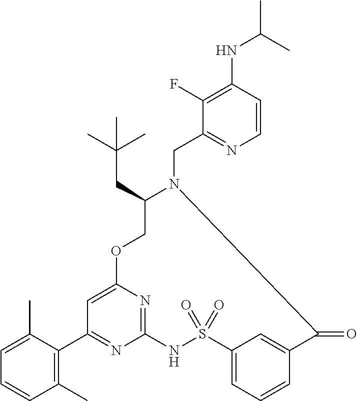 [SMILES: Cc1cccc(C)c1-c1cc2nc(n1)NS(=O)(=O)c1cccc(c1)C(=O)N(Cc1nccc(NC(C)C)c1F)[C@H](CC(C)(C)C)CO2] | +++ | +++ | |
| 1125 | 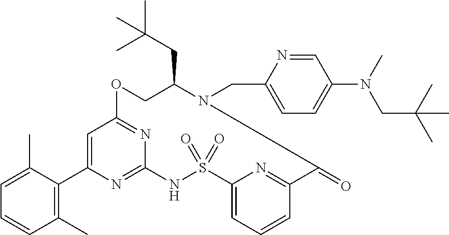 [SMILES: Cc1cccc(C)c1-c1cc2nc(n1)NS(=O)(=O)c1cccc(n1)C(=O)N(Cc1ccc(N(C)CC(C)(C)C)cn1)[C@H](CC(C)(C)C)CO2] | ++ | +++ | |
| 1126 | 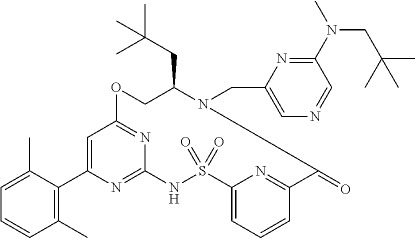 [SMILES: Cc1cccc(C)c1-c1cc2nc(n1)NS(=O)(=O)c1cccc(n1)C(=O)N(Cc1cncc(N(C)CC(C)(C)C)n1)[C@H](CC(C)(C)C)CO2] | +++ | +++ | |
| 1127 | 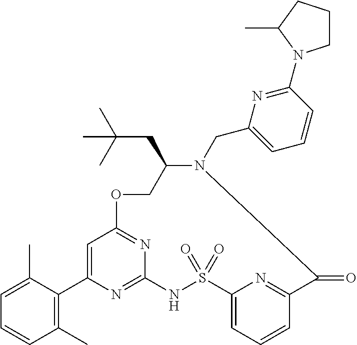 [SMILES: Cc1cccc(C)c1-c1cc2nc(n1)NS(=O)(=O)c1cccc(n1)C(=O)N(Cc1cccc(N3CCCC3C)n1)[C@H](CC(C)(C)C)CO2] | +++ | +++ | |
| 1128 | 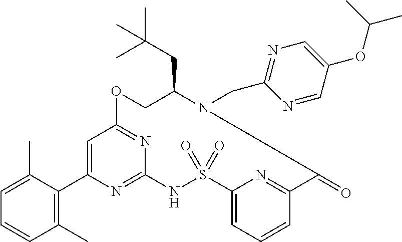 [SMILES: Cc1cccc(C)c1-c1cc2nc(n1)NS(=O)(=O)c1cccc(n1)C(=O)N(Cc1ncc(OC(C)C)cn1)[C@H](CC(C)(C)C)CO2] | +++ | +++ | |
| 1129 | 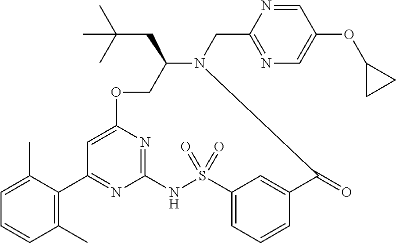 [SMILES: Cc1cccc(C)c1-c1cc2nc(n1)NS(=O)(=O)c1cccc(c1)C(=O)N(Cc1ncc(OC3CC3)cn1)[C@H](CC(C)(C)C)CO2] | +++ | +++ | |
| 1114 | 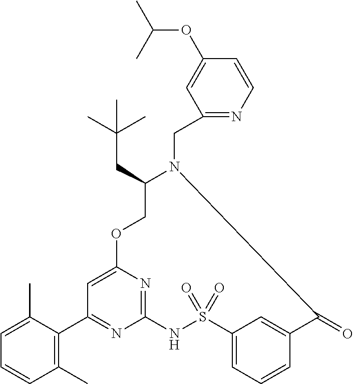 [SMILES: Cc1cccc(C)c1-c1cc2nc(n1)NS(=O)(=O)c1cccc(c1)C(=O)N(Cc1cc(OC(C)C)ccn1)[C@H](CC(C)(C)C)CO2] | | | +++ |
| 1111 | 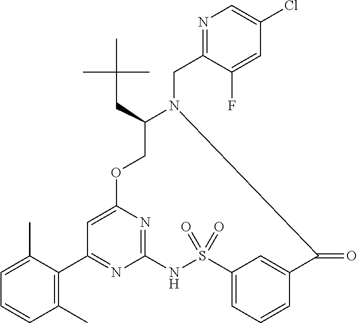 [SMILES: Cc1cccc(C)c1-c1cc2nc(n1)NS(=O)(=O)c1cccc(c1)C(=O)N(Cc1ncc(Cl)cc1F)[C@H](CC(C)(C)C)CO2] | +++ | +++ | |
| 1113 | 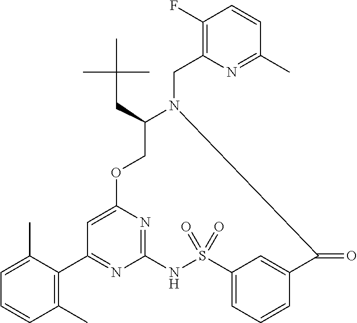 [SMILES: Cc1ccc(F)c(CN2C(=O)c3cccc(c3)S(=O)(=O)Nc3nc(cc(-c4c(C)cccc4C)n3)OC[C@H]2CC(C)(C)C)n1] | +++ | +++ | |
| 1112 | 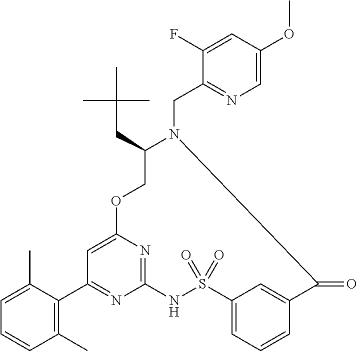 [SMILES: COc1cnc(CN2C(=O)c3cccc(c3)S(=O)(=O)Nc3nc(cc(-c4c(C)cccc4C)n3)OC[C@H]2CC(C)(C)C)c(F)c1] | +++ | +++ | |
| 1124 | 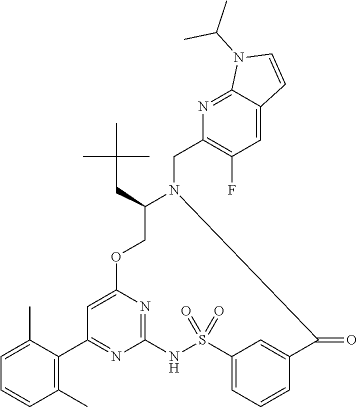 [SMILES: Cc1cccc(C)c1-c1cc2nc(n1)NS(=O)(=O)c1cccc(c1)C(=O)N(Cc1nc3c(ccn3C(C)C)cc1F)[C@H](CC(C)(C)C)CO2] | +++ | +++ | |
| 1131 | 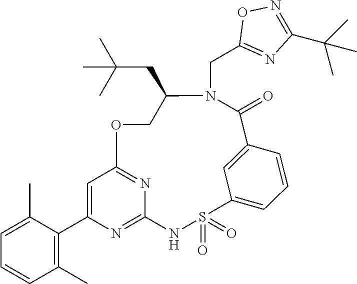 [SMILES: Cc1cccc(C)c1-c1cc2nc(n1)NS(=O)(=O)c1cccc(c1)C(=O)N(Cc1nc(C(C)(C)C)no1)[C@H](CC(C)(C)C)CO2] | | | +++ |
| 1130 | 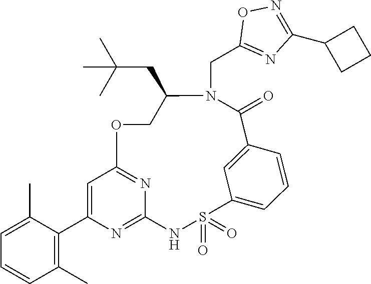 [SMILES: Cc1cccc(C)c1-c1cc2nc(n1)NS(=O)(=O)c1cccc(c1)C(=O)N(Cc1nc(C3CCC3)no1)[C@H](CC(C)(C)C)CO2] | | | +++ |
| 1117 | 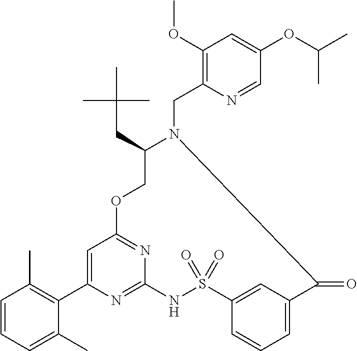 [SMILES: COc1cc(OC(C)C)cnc1CN1C(=O)c2cccc(c2)S(=O)(=O)Nc2nc(cc(-c3c(C)cccc3C)n2)OC[C@H]1CC(C)(C)C] | +++ | +++ | |
| 1144 | 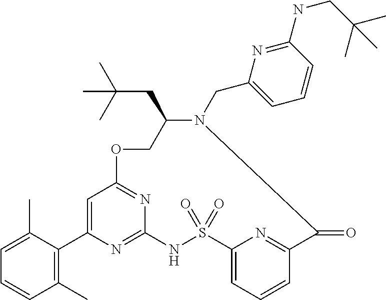 [SMILES: Cc1cccc(C)c1-c1cc2nc(n1)NS(=O)(=O)c1cccc(n1)C(=O)N(Cc1cccc(NCC(C)(C)C)n1)[C@H](CC(C)(C)C)CO2] | +++ | +++ | |
| 1142 | 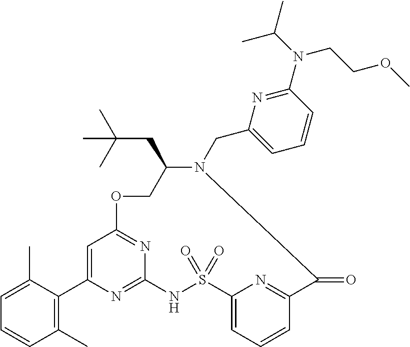 [SMILES: COCCN(c1cccc(CN2C(=O)c3cccc(n3)S(=O)(=O)Nc3nc(cc(-c4c(C)cccc4C)n3)OC[C@H]2CC(C)(C)C)n1)C(C)C] | ++ | +++ | |
| 1143 | 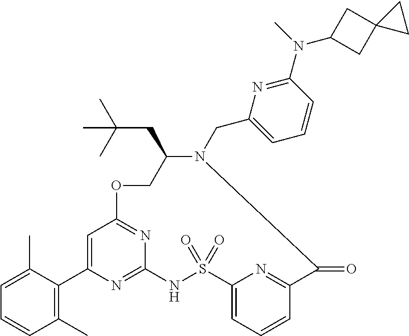 [SMILES: Cc1cccc(C)c1-c1cc2nc(n1)NS(=O)(=O)c1cccc(n1)C(=O)N(Cc1cccc(N(C)C3CC4(CC4)C3)n1)[C@H](CC(C)(C)C)CO2] | +++ | +++ | |
| 1141 | 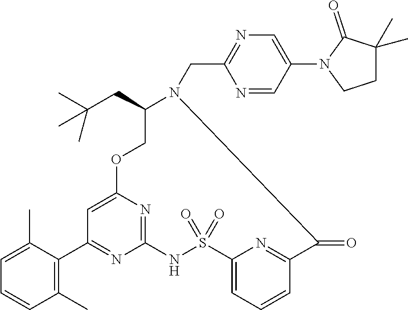 [SMILES: Cc1cccc(C)c1-c1cc2nc(n1)NS(=O)(=O)c1cccc(n1)C(=O)N(Cc1ncc(N3CCC(C)(C)C3=O)cn1)[C@H](CC(C)(C)C)CO2] | +++ | +++ | |
| 1156 | 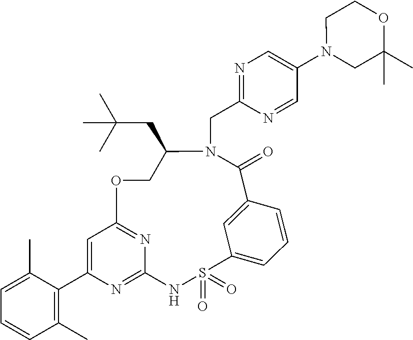 [SMILES: Cc1cccc(C)c1-c1cc2nc(n1)NS(=O)(=O)c1cccc(c1)C(=O)N(Cc1ncc(N3CCOC(C)(C)C3)cn1)[C@H](CC(C)(C)C)CO2] | +++ | +++ | |
| 1139 | 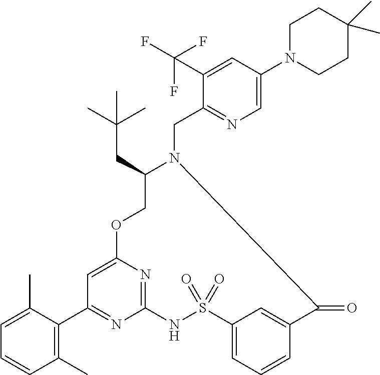 [SMILES: Cc1cccc(C)c1-c1cc2nc(n1)NS(=O)(=O)c1cccc(c1)C(=O)N(Cc1ncc(N3CCC(C)(C)CC3)cc1C(F)(F)F)[C@H](CC(C)(C)C)CO2] | ++ | +++ | |
| 1138 | 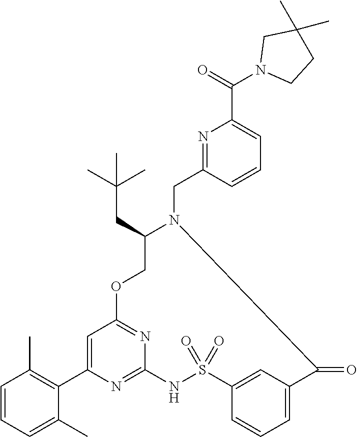 [SMILES: Cc1cccc(C)c1-c1cc2nc(n1)NS(=O)(=O)c1cccc(c1)C(=O)N(Cc1cccc(C(=O)N3CCC(C)(C)C3)n1)[C@H](CC(C)(C)C)CO2] | +++ | +++ | |
| 1134 | 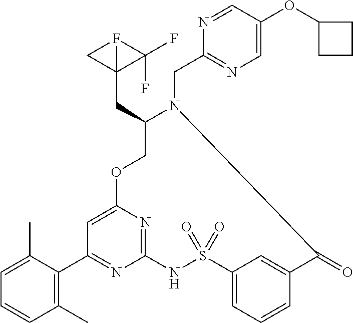 | +++ | +++ | |
| 1133 | 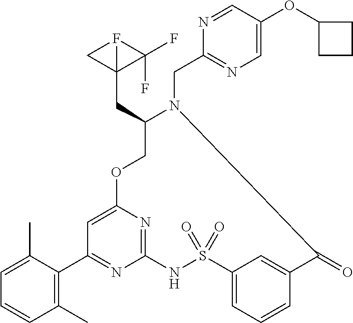 | +++ | +++ | |
| 1155 | 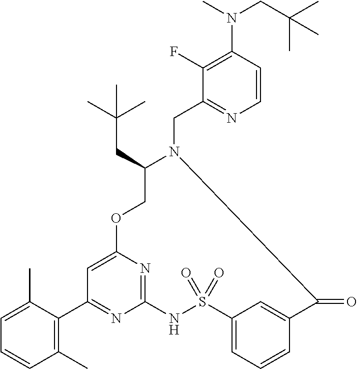 [SMILES: Cc1cccc(C)c1-c1cc2nc(n1)NS(=O)(=O)c1cccc(c1)C(=O)N(Cc1nccc(N(C)CC(C)(C)C)c1F)[C@H](CC(C)(C)C)CO2] | ++ | +++ | |
| 1149 | 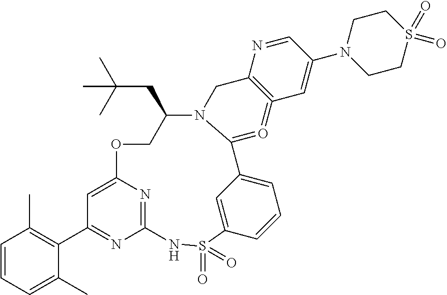 | +++ | +++ | |
| 1147 | 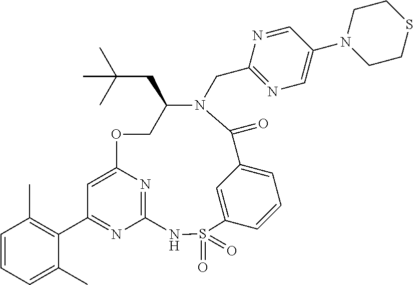 [SMILES: Cc1cccc(C)c1-c1cc2nc(n1)NS(=O)(=O)c1cccc(c1)C(=O)N(Cc1ncc(N3CCSCC3)cn1)[C@H](CC(C)(C)C)CO2] | +++ | +++ | |
| 1146 | 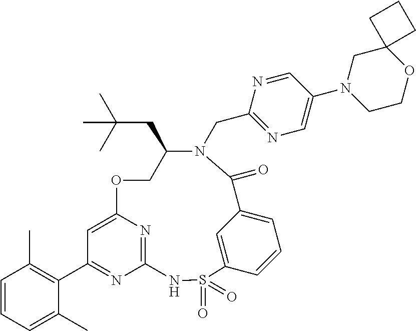 [SMILES: Cc1cccc(C)c1-c1cc2nc(n1)NS(=O)(=O)c1cccc(c1)C(=O)N(Cc1ncc(N3CCOC4(CCC4)C3)cn1)[C@H](CC(C)(C)C)CO2] | +++ | +++ | |
| 1145 | 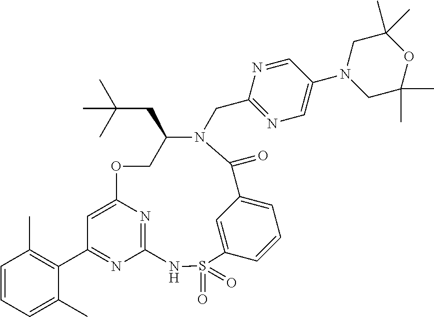 [SMILES: Cc1cccc(C)c1-c1cc2nc(n1)NS(=O)(=O)c1cccc(c1)C(=O)N(Cc1ncc(N3CC(C)(C)OC(C)(C)C3)cn1)[C@H](CC(C)(C)C)CO2] | +++ | +++ | |
| 1135 | 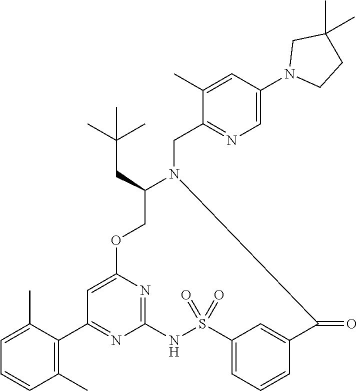 [SMILES: Cc1cc(N2CCC(C)(C)C2)cnc1CN1C(=O)c2cccc(c2)S(=O)(=O)Nc2nc(cc(-c3c(C)cccc3C)n2)OC[C@H]1CC(C)(C)C] | +++ | +++ | |
| 1153 | 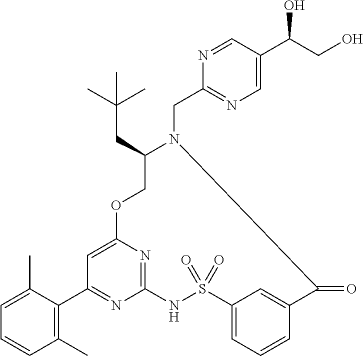 [SMILES: Cc1cccc(C)c1-c1cc2nc(n1)NS(=O)(=O)c1cccc(c1)C(=O)N(Cc1ncc([C@@H](O)CO)cn1)[C@H](CC(C)(C)C)CO2] | ++ | +++ | |
| 1152 | 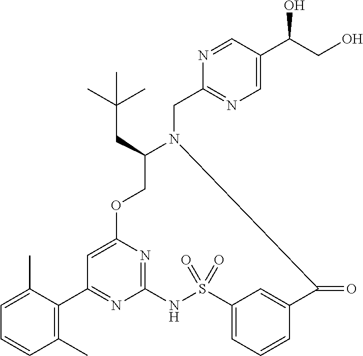 [SMILES: Cc1cccc(C)c1-c1cc2nc(n1)NS(=O)(=O)c1cccc(c1)C(=O)N(Cc1ncc([C@@H](O)CO)cn1)[C@H](CC(C)(C)C)CO2] | +++ | +++ | |
| 1151 | 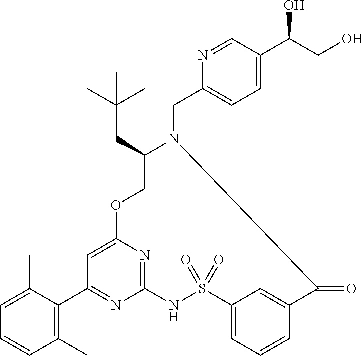 [SMILES: Cc1cccc(C)c1-c1cc2nc(n1)NS(=O)(=O)c1cccc(c1)C(=O)N(Cc1ccc([C@@H](O)CO)cn1)[C@H](CC(C)(C)C)CO2] | ++ | +++ | |
| 1150 | 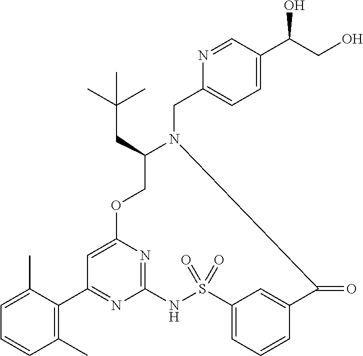 [SMILES: Cc1cccc(C)c1-c1cc2nc(n1)NS(=O)(=O)c1cccc(c1)C(=O)N(Cc1ccc([C@@H](O)CO)cn1)[C@H](CC(C)(C)C)CO2] | +++ | +++ | |
| 1154 | 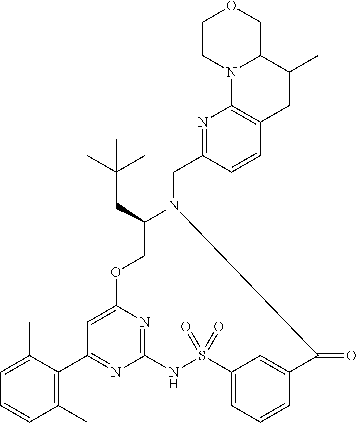 [SMILES: Cc1cccc(C)c1-c1cc2nc(n1)NS(=O)(=O)c1cccc(c1)C(=O)N(Cc1ccc3c(n1)N1CCOCC1C(C)C3)[C@H](CC(C)(C)C)CO2] | +++ | +++ | |
| 1148 | 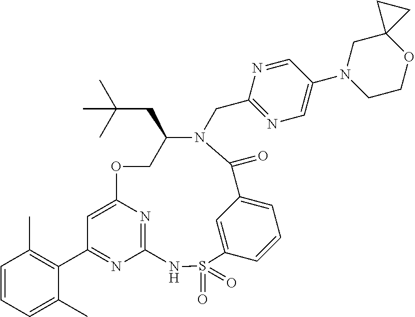 [SMILES: Cc1cccc(C)c1-c1cc2nc(n1)NS(=O)(=O)c1cccc(c1)C(=O)N(Cc1ncc(N3CCOC4(CC4)C3)cn1)[C@H](CC(C)(C)C)CO2] | +++ | +++ | |
| 1137 | 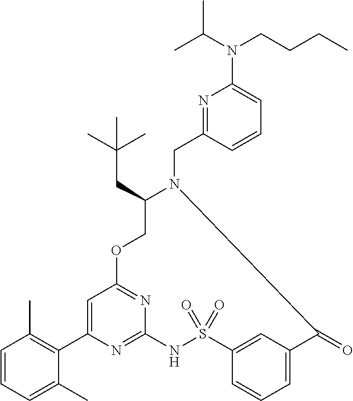 [SMILES: CCCCN(c1cccc(CN2C(=O)c3cccc(c3)S(=O)(=O)Nc3nc(cc(-c4c(C)cccc4C)n3)OC[C@H]2CC(C)(C)C)n1)C(C)C] | +++ | +++ | |
| 1136 | 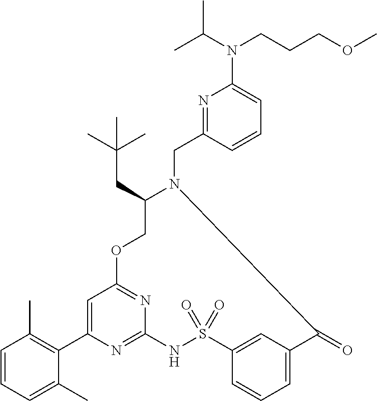 [SMILES: COCCCN(c1cccc(CN2C(=O)c3cccc(c3)S(=O)(=O)Nc3nc(cc(-c4c(C)cccc4C)n3)OC[C@H]2CC(C)(C)C)n1)C(C)C] | +++ | +++ | |
| 1140 | 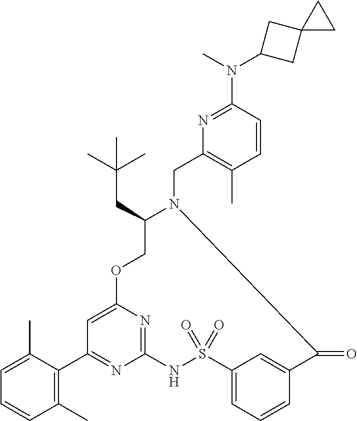 [SMILES: Cc1ccc(N(C)C2CC3(CC3)C2)nc1CN1C(=O)c2cccc(c2)S(=O)(=O)Nc2nc(cc(-c3c(C)cccc3C)n2)OC[C@H]1CC(C)(C)C] | +++ | +++ | |

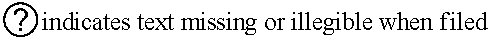

### VI. Synthesis of Compounds 1295-1972

### A. Synthesis of Common Intermediates

### Example X: Preparation of 5-(Cyclobutoxy)pyrimidine-2-carbaldehyde

### Step 1: 2-Chloro-5-(cyclobutoxy)pyrimidine

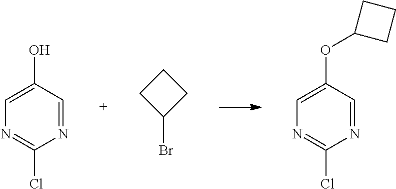 [SMILES: Oc1cnc(Cl)nc1 ; BrC1CCC1 ; Clc1ncc(OC2CCC2)cn1]

A stirred mixture of 2-chloropyrimidin-5-ol (42.6 g, 326.36 mmol), bromocyclobutane (58 g, 429.62 mmol) and potassium carbonate (113 g, 817.62 mmol) in DMF (400 mL) was heated at 80° C. for 2 h. The reaction mixture was allowed to cool to rt and poured onto water (1 L). The solids were filtered, washed with water, and dissolved in DCM (1 L). The DCM layer was dried over sodium sulfate, filtered and concentrated in vacuo to give 2-chloro-5-(cyclobutoxy)pyrimidine (42.3 g, 63%) as a white solid. ¹H NMR (500 MHz, DMSO-d₆) δ 8.42 (s, 2H), 4.84 (p, J 6.8 Hz, 1H), 2.48-2.42 (m, 2H), 2.10-2.00 (m, 2H), 1.84-1.76 (m, 1H), 1.68-1.55 (m, 1H). ESI-MS m/z calc. 184.04034, found 185.5 (M+1)⁺; Retention time: 3.72 minutes; LC method S.

### Step 2: Methyl 5-(cyclobutoxy)pyrimidine-2-carboxylate

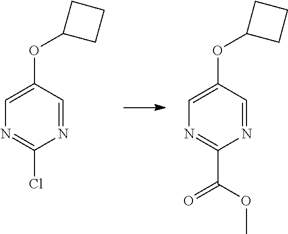

A mixture of 2-chloro-5-(cyclobutoxy)pyrimidine (23.4 g, 114.07 mmol) and Pd(dppf)₂Cl₂CH₂Cl₂ (6.5 g, 7.9595 mmol) in DMF (240 mL)/TEA (240 mL)/MeOH (240 mL) in a 2 L steel bomb with overhead mechanical stirrer was purged with carbon monoxide three times. The reaction mixture was heated to 100° C. with 120 psi of CO over one hour and retained at this temperature for one hour. Heating was turned off and the reaction mixture was allowed to cool to rt. Methanol and triethylamine was evaporated in vacuo. Water (500 mL) was added and filtered to remove catalyst residue. The cake was washed with water. The filtrate was extracted with DCM (3×500 mL). The combined organic layer was washed with brine, dried over sodium sulfate, filtered and concentrated in vacuo. The resulting brown oil was purified by flash chromatography (Silica, 120 g, loaded in DCM, eluted with 25% ethyl acetate in hexanes) to give methyl 5-(cyclobutoxy)pyrimidine-2-carboxylate (11.9 g, 48%) as a beige solid. ESI-MS m/z calc. 208.0848, found 209.1 (M+1)⁺; Retention time: 3.67 minutes; LC method S.

### Step 3: 5-(Cyclobutoxy)pyrimidine-2-carbaldehyde

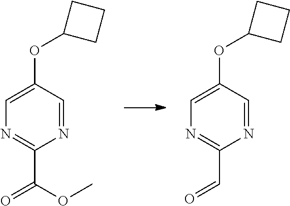

To a stirred solution of methyl 5-(cyclobutoxy)pyrimidine-2-carboxylate (30.1 g, 137.33 mmol) in THF (1 L) was added DIBAL in toluene (200 mL of 1 M, 200.00 mmol) at −78° C. over 30 minutes and the reaction was stirred for 1 h. The reaction mixture was quenched with methanol (200 mL) and water (200 mL). The dry ice bath was removed, and the reaction mixture was allowed to warm to rt. The mixture was concentrated in vacuo to remove THF and methanol. DCM (1 L) was added, and the mixture was filtered. The organic layer from the filtrate was separated and the aqueous layer was extracted with DCM (2×500 mL). The combined organic layer was dried over sodium sulfate, filtered and concentrated in vacuo. The resulting yellow solid was purified by flash chromatography (Silica, 220 g, loaded in DCM, eluted with 25% ethyl acetate in hexanes). The desired product fractions were combined and concentrated in vacuo to give 5-(cyclobutoxy)pyrimidine-2-carbaldehyde (21.5 g, 82%) as a white solid. ¹H NMR (500 MHz, DMSO-d₆) δ 9.88 (s, 1H), 8.67 (s, 2H), 5.08-4.92 (m, 1H), 2.58-2.48 (m, 2H), 2.18-2.03 (m, 2H), 1.88-1.77 (m, 1H), 1.74-1.59 (m, 1H). ESI-MS m/z calc. 178.07423, found 179.2 (M+1)⁺; Retention time: 1.38 minutes; LC method W.

### Example Y: Preparation of 6-[cyclobutyl(methyl)amino]pyrazine-2-carbaldehyde Step 1: Methyl 6-[cyclobutyl(methyl)amino]pyrazine-2-carboxylate

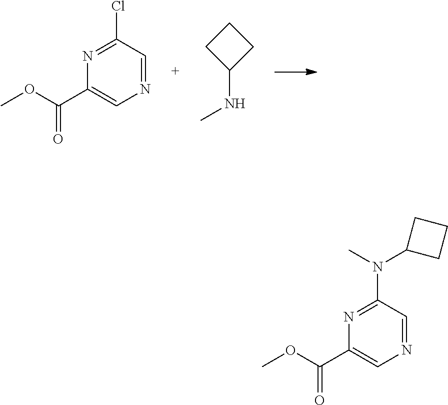

To a stirring solution of methyl 6-chloropyrazine-2-carboxylate (10.5 g, 60.845 mmol) and N-methylcyclobutanamine (hydrochloride salt) (9.46 g, 73.901 mmol) in anhydrous DMSO (150 mL) under nitrogen was added anhydrous sodium carbonate (20 g, 188.70 mmol) in one portion at room temperature. The resulting black mixture was stirred at 90° C. overnight. After cooling to room temperature, water (1000 mL) was added, and the resulting solution was extracted with EtOAc (3×300 mL). The combined organic solutions were washed with water (2×300 mL), followed by brine (300 mL), dried over anhydrous sodium sulfate, and filtered. The solvent was removed by rotary evaporation and the residue was dried in vacuo overnight yielding methyl 6-[cyclobutyl(methyl)amino]pyrazine-2-carboxylate (7.77 g, 58%) as an amber oil. The crude was used in the next Step without further purification. ¹H NMR (500 MHz, CDCl₃) δ 8.48 (s, 1H), 8.14 (s, 1H), 4.76-4.65 (m, 1H), 3.96 (s, 3H), 3.11 (s, 3H), 2.37-2.27 (m, 2H), 2.26-2.14 (m, 2H), 1.82-1.70 (m, 2H). ESI-MS m/z calc. 221.11642, found 222.2 (M+1)⁺; Retention time: 2.43 minutes; LC method T.

### Step 2: [6-[Cyclobutyl(methyl)amino]pyrazin-2-yl]methanol

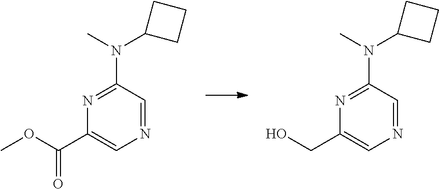 [SMILES: COC(=O)c1cncc(N(C)C2CCC2)n1 ; CN(c1cncc(CO)n1)C1CCC1]

A solution of methyl 6-[cyclobutyl(methyl)amino]pyrazine-2-carboxylate (7.77 g, 35.118 mmol) in MeOH (200 mL) was cooled to 0° C. using an ice bath. Sodium borohydride (13.3 g, 351.55 mmol) was then added in portions over 15 min at the same temperature. The reaction mixture was stirred for 1 h and then warmed to room temperature and stirred for 7 h. The reaction mixture was quenched with water (100 mL). MeOH was removed by rotary evaporation and the remaining aqueous layer was further diluted with water (200 mL), saturated with sodium chloride, and extracted with DCM (100 mL×5). The combined organic solutions were dried over anhydrous sodium sulfate and filtered. The solvent was removed by rotary evaporation and the residue was dried in vacuo for 5 h yielding [6-[cyclobutyl(methyl)amino]pyrazin-2-yl]methanol (5.45 g, 79%) as a yellow oil. The crude was used in the next Step without further purification. ESI-MS m/z calc. 193.1215, found 194.1 (M+1)⁺; Retention time: 1.41 minutes; LC method T.

### Step 3: 6-[Cyclobutyl(methyl)amino]pyrazine-2-carbaldehyde

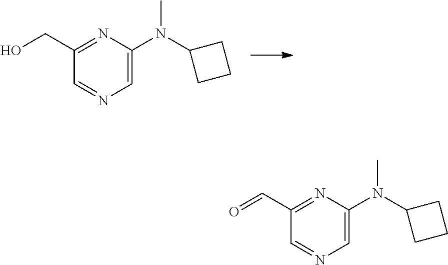

A solution of [6-[cyclobutyl(methyl)amino]pyrazin-2-yl]methanol (5.45 g, 27.638 mmol) in anhydrous DCM (250 mL) was added with DMP (14.2 g, 33.479 mmol) in portions over 5 min under nitrogen. The resulting amber solution was stirred at room temperature overnight. LCMS indicated incomplete oxidation. More DMP (3.6 g, 8.4877 mmol) was added and stirring was continued for 3 h. The reaction mixture was added with a saturated aqueous sodium bicarbonate solution (200 mL) and stirred for 15 min. The organic layer was separated and washed further with saturated aqueous sodium bicarbonate solution (2×200 mL). The organic layer was dried over anhydrous sodium sulfate and filtered. The solvent was removed by rotary evaporation and the crude aldehyde was purified by silica flash chromatography (330 g, dry loaded, eluting from 0 to 40% EtOAc in hexanes over a 70 min gradient). The fractions were combined and concentrated under reduced pressure and the residue was further dried in vacuo overnight yielding 6-[cyclobutyl(methyl)amino]pyrazine-2-carbaldehyde (3.7 g, 67%) as an orange liquid. ¹H NMR (500 MHz, DMSO-d₆) δ 9.90 (s, 1H), 8.40 (s, 1H), 8.27 (s, 1H), 4.89-4.80 (m, 1H), 3.08 (s, 3H), 2.26-2.16 (m, 4H), 1.75-1.61 (m, 2H). ESI-MS m/z calc. 191.10587, found 192.1 (M+1)⁺; Retention time: 2.19 minutes; LC method W.

### B. Synthesis of Compounds 1295 to 1972

### Example 137: Preparation of Compound 1295

### Step 1: 5-(3,3-Dimethylazetidin-1-yl)pyrimidine-2-carbonitrile

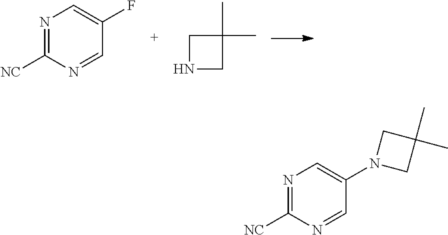

To a stirring solution of 5-fluoropyrimidine-2-carbonitrile (23.3 g, 181.72 mmol) and 3,3-dimethylazetidine (hydrochloride salt) (30.18 g, 240.73 mmol) in anhydrous DMF (360 mL) at 0° C. under nitrogen was added cesium carbonate (60 g, 184.15 mmol) in portions over 15 min. The resulting mixture was stirred at the same temperature for 1 h and then warmed up to room temperature and stirred overnight. The reaction mixture was quenched with ice-cold water (1000 mL). Ethyl acetate (1000 mL) was added, and the mixture was allowed to warm up to room temperature. Two layers were separated, and the aqueous layer was extracted with ethyl acetate (3×500 mL). The combined organic layers were washed with water (3×700 mL), brine (700 mL), dried over anhydrous sodium sulfate and concentrated in vacuo to afford 5-(3,3-dimethylazetidin-1-yl)pyrimidine-2-carbonitrile (34.86 g, 100%) as an off white solid. The crude product was used in the next step without further purification. ESI-MS m/z calc. 188.1062, found 189.1 (M+1)⁺; Retention time: 2.55 minutes; LC method T.

### Step 2: 5-(3,3-Dimethylazetidin-1-yl)pyrimidine-2-carbaldehyde

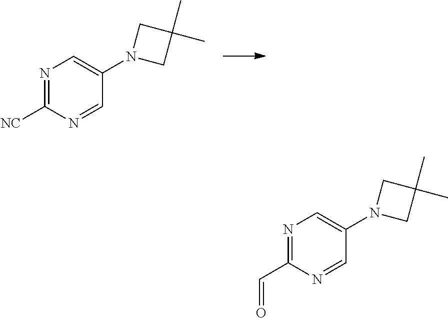

A stirring solution of 5-(3,3-dimethylazetidin-1-yl)pyrimidine-2-carbonitrile (11.12 g, 59.077 mmol) in anhydrous THF (300 mL) under nitrogen was cooled to −78° C. using a dry ice-acetone bath. To the cloudy reaction mixture was added diisobutylaluminum hydride in toluene (80 mL of 1 M, 80.000 mmol) dropwise over 30 min. The reaction mixture was stirred at −78° C. for 2 h. The reaction mixture was quenched with saturated aqueous sodium potassium tartrate solution (150 mL) and stirred at this temperature for 15 minutes. Another portion of saturated aqueous sodium potassium tartrate solution (200 mL) and ethyl acetate (300 mL) were added. The reaction mixture was allowed to warm up to room temperature over 2 h while stirring. Two layers were separated, and the aqueous layer was extracted with ethyl acetate (3×100 mL). The combined organic layers were washed with brine (300 mL), dried over anhydrous sodium sulfate, and concentrated. The crude product was purified by silica flash chromatography (330 g, dry loaded, eluted from 0 to 5% MeOH in dichloromethane over a 60 min gradient). The fractions were combined and concentrated by rotary evaporation and the residue was dried in vacuo overnight yielding 5-(3,3-dimethylazetidin-1-yl)pyrimidine-2-carbaldehyde (5.9 g, 51%) as an orange solid. ¹H NMR (500 MHz, DMSO-d₆) δ 9.74 (s, 1H), 8.12 (s, 2H), 3.83 (s, 4H), 1.31 (s, 6H). ESI-MS m/z calc. 191.10587, found 192.1 (M+1)⁺; Retention time: 1.8 minutes; LC method W.

### Step 3: (2R)-2-[[5-(3,3-Dimethylazetidin-1-yl)pyrimidin-2-yl]methylamino]-4,4-dimethyl-pentan-1-ol

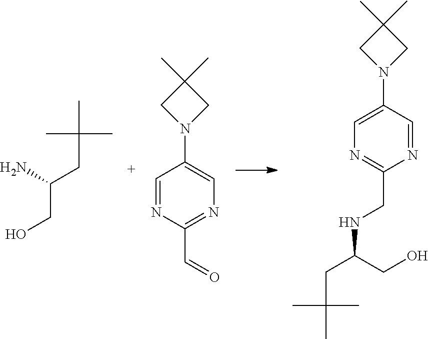

(2R)-2-Amino-4,4-dimethyl-pentan-1-ol (78 g, 594.4 mmol) and three different baches of 5-(3,3-dimethylazetidin-1-yl)pyrimidine-2-carbaldehyde (57.7 g, 301.7 mmol, 36.3 g, 189.8 mmol, and 19.7 g, 103.0 mmol) were suspended in DCE (1.17 L) and glacial acetic acid (39 mL, 685.8 mmol) was added to give a clear orange solution. The solution was stirred at room temperature for 0.5 h and slightly cooled in a cold-water bath. At an internal temperature of 16° C., sodium triacetoxyborohydride (164 g, 773.8 mmol) was added over 5 minutes, while stirring in the cold water (10° C.) which resulted in a slight exotherm with the internal temperature raising to 35° C. The mixture was stirred at room temperature for 3.5 hours to give an orange solution. The reaction was quenched by dropwise addition of aqueous HCl (1.49 L of 1 M, 1.490 mol) at room temperature and stirred for 0.5 hour. Then NaOH (350 g of 50% w/w, 4.375 mol) was added dropwise under cooling, keeping the internal temperature between 15 and 20° C. (final pH ˜12). The phases were separated, and the aqueous phase extracted twice more with DCE (2×150 mL). The combined organic phases were washed once with water (500 mL) and the organic phase was dried over magnesium sulfate, filtered, and evaporated. The crude (189 g, solid mass) was treated with heptane (1.2 L) and heated to 95° C. (on rotary evaporator) to give a solution. The hot solution was cooled to 75° C., seeded and rotated in the cooling water bath for 3 hours to give a thick suspension. The suspension was filtered, washed with cold heptane and dried to give (2R)-2-[[5-(3,3-dimethylazetidin-1-yl)pyrimidin-2-yl]methylamino]-4,4-dimethyl-pentan-1-ol as a pink solid (167 g, 92%). ¹H NMR (400 MHz, CDCl₃) δ 7.89 (s, 2H), 4.04 (d, J 15.4 Hz, 1H), 3.93 (d, J 15.4 Hz, 1H), 3.65 (s, 4H), 3.61 (dd, J 10.9, 3.7 Hz, 1H), 3.31 (dd, J 10.9, 6.7 Hz, 1H), 2.77 (dtd, J 6.8, 5.0, 3.7 Hz, 1H), 1.40-1.36 (m, 2H), 1.35 (s, 6H), 0.94 (s, 9H). ESI-MS m/z calc. 306.24197, found 307.0 (M+1)⁺; Retention time: 1.46 minutes; LC method 1A.

### Step 4: 3-[[4-[(2R)-2-[[5-(3,3-Dimethylazetidin-1-yl)pyrimidin-2-yl]methylamino]-4,4-dimethyl-pentoxy]-6-(2,6-dimethylphenyl)pyrimidin-2-yl]sulfamoyl]benzoic acid

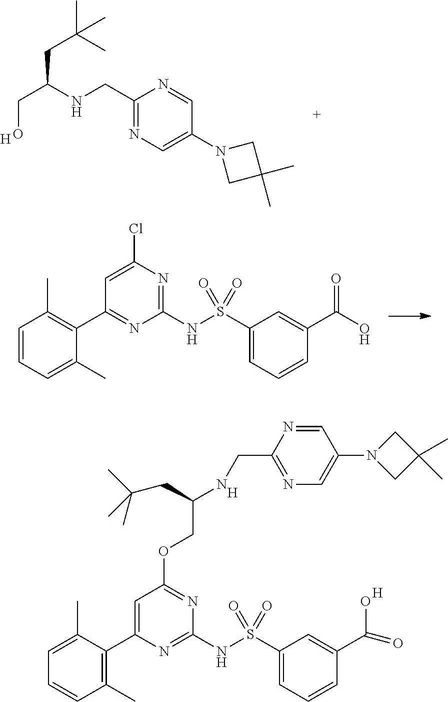

Sodium tert-butoxide (25.1 g, 261.2 mmol) was added to a suspension of (2R)-2-[[5-(3,3-dimethylazetidin-1-yl)pyrimidin-2-yl]methylamino]-4,4-dimethyl-pentan-1-ol (20 g, 65.26 mmol) and 3-[[4-chloro-6-(2,6-dimethylphenyl)pyrimidin-2-yl]sulfamoyl]benzoic acid (27.3 g, 65.33 mmol) in 2-MeTHF (160 mL) in an ice bath at 0° C. After warming up to 37° C., the reaction mixture turned into a thick gel. It was diluted with more MeTHF to a final reaction volume of 450 mL, then heated to 51° C. The mixture was cooled to 0° C. in ice bath, then poured into cold HCl (196 mL of 2 M, 392.0 mmol). The layers were separated, the water layer was extracted with MeTHF (100 mL), and the combined organic layers was washed with brine (100 mL). The organic layer was again washed with brine (100 mL), then dried with magnesium sulfate and concentrated at 25° C. down to a total weight of 147 g. EtOAc (470 mL) was added while stirring, the resulting slurry was stirred overnight, then the product was collected by filtration to give 3-[[4-[(2R)-2-[[5-(3,3-dimethylazetidin-1-yl)pyrimidin-2-yl]methylamino]-4,4-dimethyl-pentoxy]-6-(2,6-dimethylphenyl)pyrimidin-2-yl]sulfamoyl]benzoic acid (hydrochloride salt) (42.34 g, 83%), ¹H NMR (400 MHz, DMSO-d₆) δ 13.26 (s, 2H), 12.08 (s, 1H), 9.36 (s, 2H), 8.45 (s, 1H), 8.22-8.07 (m, 2H), 8.03 (s, 2H), 7.69 (t, J 7.8 Hz, 1H), 7.26 (t, J 7.6 Hz, 1H), 7.13 (d, J 7.6 Hz, 2H), 6.30 (s, 1H), 4.46-4.18 (m, 4H), 3.66 (s, 5H), 2.01 (s, 5H), 1.73-1.61 (m, 2H), 1.29 (s, 6H), 0.91 (s, 9H). ESI-MS m/z calc. 687.3203, found 688.3 (M+1)⁺; Retention time: 2.09 minutes; LC method 1A.

### Step 5: (11R)-12-[[5-(3,3-Dimethylazetidin-1-yl)pyrimidin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

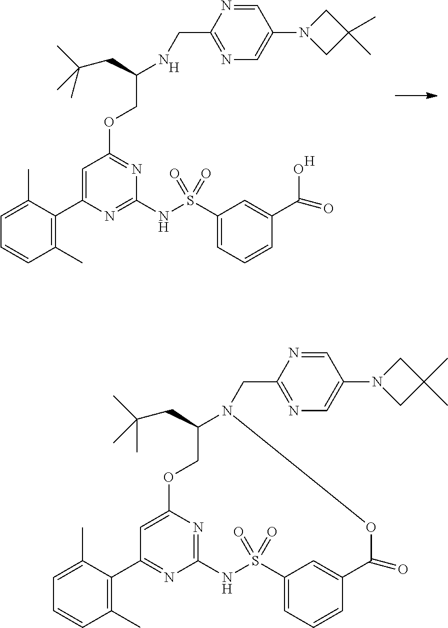

T3P (102 mL of 50% w/w in EtOAc, 171.3 mmol) was added to a suspension of 3-[[4-[(2R)-2-[[5-(3,3-dimethylazetidin-1-yl)pyrimidin-2-yl]methylamino]-4,4-dimethyl-pentoxy]-6-(2,6-dimethylphenyl)pyrimidin-2-yl]sulfamoyl]benzoic acid (hydrochloride salt) (42.38 g, 54.36 mmol) in acetonitrile (1575 mL) at 0° C., followed by DIPEA (58.7 mL, 337.0 mmol). The mixture was stirred at 0° C. for 2.5 hours, then approximately 90% of the acetonitrile was removed under vacuum at 25° C. EtOAc (500 mL) was added, followed by a solution of citric Acid (34.0 g, 177.0 mmol) in water (750 mL). The layers were separated, and the organic layer was washed with a solution of citric Acid (5.3 g, 27.59 mmol) in water (200 mL) and brine (200 mL). The organic layer was then washed with a solution of potassium carbonate (15.1 g, 109.3 mmol) in water (100 mL) and brine (200 mL), dried with magnesium sulfate, and concentrated under vacuum at 45° C. to give 38.35 g light orange solid (crude potassium salt of product). The crude 38.35 g was suspended in DCM (300 mL), then a solution of citric Acid (26.2 g, 136.4 mmol) in water (273 mL) was added and stirred vigorously for 15 minutes. The layers were separated, and the organic layer was dried with magnesium sulfate and concentrated to give 38.3 g. Boiling acetonitrile (219 mL) was added to the solid, and the mixture was heated at reflux for 10 minutes, then stirred vigorously overnight at room temperature. The product was collected by filtration, rinsed with cold acetonitrile, then dried under vacuum at 40° C. to give 35 g. 22.8 g of the material that was recrystallized from EtOH (228 mL) to give (11R)-12-[[5-(3,3-dimethylazetidin-1-yl)pyrimidin-2-yl]methyl]-6-(2,6-dimethylphenyl)-11-(2,2-dimethylpropyl)-2,2-dioxo-9-oxa-2λ⁶-thia-3, 5,12,19-tetrazatricyclo[12.3.1.14,8]nonadeca-1(18),4(19),5,7,14,16-hexaen-13-one (19.45 g, 53%), ¹H NMR (400 MHz, Chloroform-d) δ 9.30 (s, 1H), 9.03-8.86 (m, 1H), 8.07 (d, J 8.0, 1.4 Hz, 1H), 7.92 (s, 2H), 7.83 (d, J 7.7, 1.4 Hz, 1H), 7.62 (t, J 7.8 Hz, 1H), 7.20 (t, J 7.6 Hz, 1H), 7.03 (d, J 7.6 Hz, 2H), 6.17 (s, 1H), 5.51 (dd, J 11.3, 4.3 Hz, 1H), 5.27 (d, J 16.3 Hz, 1H), 4.32 (d, J 16.4 Hz, 1H), 4.22-4.01 (m, 1H), 3.78 (t, J 11.5 Hz, 1H), 3.65 (s, 4H), 1.98 (s, 6H), 1.78-1.70 (m, 1H), 1.50 (d, J 15.2, 1.8 Hz, 1H), 1.34 (s, 6H), 0.65 (s, 9H). ESI-MS m/z calc. 669.30975, found 670.6 (M+1)⁺; Retention time: 2.68 minutes; LC method 1A.

### Example 138: Preparation of Compound 1296

### Step 1: Methyl 2-(tert-butoxycarbonylamino)-5,5-dimethyl-hex-2-enoate

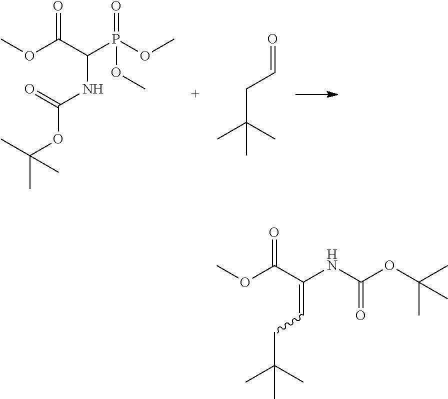

To a stirred solution of methyl 2-(tert-butoxycarbonylamino)-2-dimethoxyphosphoryl-acetate (16.4 g, 55.174 mmol) and DBU (8.0422 g, 7.9 mL, 52.827 mmol) in DCM (100 mL) was added at 0° C. (ice bath) 3,3-dimethylbutyraldehyde (5.0274 g, 6.3 mL, 50.194 mmol). The reaction mixture was stirred at room temperature for 16 h. Aqueous HCl (1 N) (100 mL) was added, and the phases were separated. The aqueous layer was washed with DCM (2×100 mL). The combined organic layers were dried with sodium sulfate, filtered, and concentrated under reduced pressure. The crude residue was purified by 50 g silica gel pad using a gradient of 15% EtOAc in heptanes to afford methyl 2-(tert-butoxycarbonylamino)-5,5-dimethyl-hex-2-enoate (13.6 g, 95%) as a clear oil that crystallized to a white solid. ¹H NMR (400 MHz, CDCl₃) δ 6.66 (t, J 7.6 Hz, 1H), 5.86 (br. s, 1H), 3.79 (s, 3H), 2.12 (d, J 7.6 Hz, 2H), 1.47 (s, 9H), 0.95 (s, 9H).

### Step 2: Methyl (2R)-2-(tert-butoxycarbonylamino)-5,5-dimethyl-hexanoate

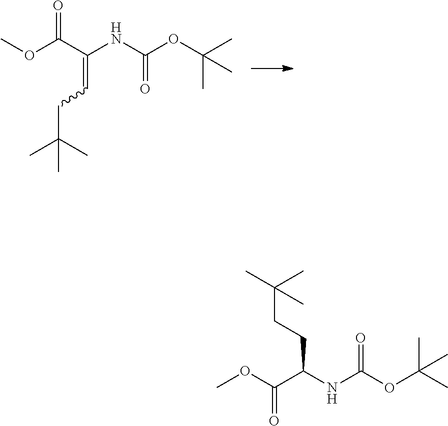 [SMILES: COC(=O)C(=CCC(C)(C)C)NC(=O)OC(C)(C)C ; COC(=O)[C@@H](CCC(C)(C)C)NC(=O)OC(C)(C)C]

To a solution of methyl 2-(tert-butoxycarbonylamino)-5,5-dimethyl-hex-2-enoate (13.630 g, 50.230 mmol) in ethanol (184 mL) and 1,4-dioxane (61 mL) was bubbled nitrogen for 5 min. Then, 1,2-bis[(2R,5R)-2,5-diethylphospholano]benzene(1,5-cyclooctadiene)rhodium(I) trifluoromethanesulfonate (363 mg, 0.5023 mmol) was added and the mixture was put in an ultrasound bath for 5 min under nitrogen. The reaction mixture was then purged with nitrogen gas (3×30 psi) then purged with hydrogen (3×50 psi). A pressure of 50 psi (3.5 bar) of hydrogen pressure was maintained and the reaction was stirred at room temperature for 16 h, at which time the volatiles were removed under reduced pressure and the residue was passed through a plug of silica gel (80 g) using an eluent of 15% EtOAc in heptanes to afford methyl (2R)-2-(tert-butoxycarbonylamino)-5,5-dimethyl-hexanoate (14 g, 97%) as a colorless oil. ¹H NMR (400 MHz, CDCl₃) δ 5.00 (d, J 7.3 Hz, 1H), 4.27 (d, J 5.4 Hz, 1H), 3.74 (s, 3H), 1.87-1.71 (m, 1H), 1.64-1.54 (m, 1H), 1.45 (s, 9H), 1.28-1.15 (m, 2H), 0.87 (s, 9H). ESI-MS m/z calc. 273.194, found 296.2 (M+23)⁺; Retention time: 4.58 minutes; LC method Y.

### Step 3: tert-Butyl N-[(1R)-1-(hydroxymethyl)-4,4-dimethyl-pentyl]carbamate

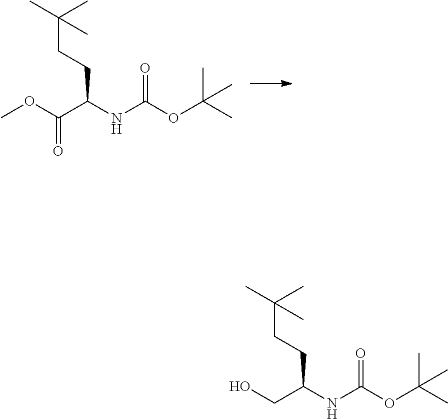 [SMILES: COC(=O)[C@@H](CCC(C)(C)C)NC(=O)OC(C)(C)C ; CC(C)(C)CC[C@H](CO)NC(=O)OC(C)(C)C]

To a solution of methyl (2R)-2-(tert-butoxycarbonylamino)-5,5-dimethyl-hexanoate (14 g, 48.652 mmol) in THF (145 mL) was added LiBH₄ (2 M solution in THF) (61 mL of 2 M, 122.00 mmol) (no exotherm observed). The reaction mixture was stirred at room temperature for 2.5 h. The reaction mixture was then poured slowly over a saturated aqueous solution of NH₄Cl (50 mL) at 0° C. (strong evolution of gas, but no exotherm). The product was extracted with EtOAc (3×150 mL). The combined organic layers were washed with brine (150 mL), dried with sodium sulfate, filtered and concentrated under reduced pressure to afford crude product tert-butyl N-[(1R)-1-(hydroxymethyl)-4,4-dimethyl-pentyl]carbamate (13.23 g, 89%) as a clear oil. ESI-MS m/z calc. 245.1991, found 268.2 (M+23)⁺; Retention time: 1.8 minutes. ¹H NMR (400 MHz, CDCl₃) δ 4.62 (br. s, 1H), 3.68 (d, J 7.1 Hz, 1H), 3.55 (d, J 8.1 Hz, 2H), 2.57 (br. s, 1H), 1.55-1.29 (m, 11H), 1.28-1.19 (m, 2H), 0.88 (s, 9H); LC method X.

### Step 4: (2R)-2-Amino-5,5-dimethyl-hexan-1-ol

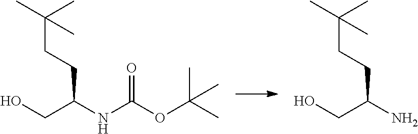

To a solution of tert-butyl N-[(1R)-1-(hydroxymethyl)-4,4-dimethyl-pentyl]carbamate (13.23 g, 43.460 mmol) in 1,4-dioxane (140 mL) was added hydrogen chloride (4 N in 1,4-dioxane) (63 mL of 4 M, 252.00 mmol). The reaction mixture was stirred at room temperature for 16 h then the mixture was evaporated to dryness under reduced pressure. The residue was triturated in THF then filtered to give (2R)-2-amino-5,5-dimethyl-hexan-1-ol (hydrochloride salt) (8.137 g, 98%) as a white solid. ¹H NMR (400 MHz, DMSO-d₆) δ 7.97 (br. s, 3H), 5.28 (br. s, 1H), 3.58 (dd, J 11.4, 3.5 Hz, 1H), 3.44 (dd, J 11.4, 6.2 Hz, 1H), 3.02-2.89 (m, 1H), 1.58-1.43 (m, 2H), 1.30-1.13 (m, 2H), 0.86 (s, 9H). ESI-MS m/z calc. ESI-MS m/z calc. 145.14667, found 146.3 (M+1)⁺; Retention time: 1.78 minutes; LC method Y.

### Step 5: 2-Chloro-5-isopropoxy-pyrimidine

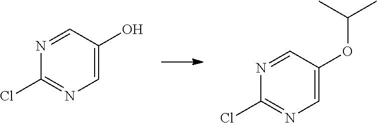

To a solution of 2-chloropyrimidin-5-ol (15 g, 114.91 mmol) in DMF (150 mL) was added potassium carbonate (32 g, 231.54 mmol) then 2-iodopropane (29.803 g, 17.5 mL, 175.32 mmol) and the mixture was heated at 50° C. for 1 h. The reaction was cooled to room temperature and diluted with diethyl ether (1500 mL), washed with a mixture of brine (300 mL) and water (300 mL), then brine (2×300 mL) again. The organics were dried over sodium sulfate, filtered and concentrated in vacuo to provide 2-chloro-5-isopropoxy-pyrimidine (18.5 g, 91%) as flaky white crystals. ¹H NMR (500 MHz, Chloroform-d) δ 8.25 (s, 2H), 4.59 (hept, J 6.1 Hz, 1H), 1.38 (d, J 6.1 Hz, 6H). ESI-MS m/z calc. 172.04034, found 173.4 (M+1)⁺; Retention time: 2.2 minutes; LC method T.

### Step 6: Methyl 5-isopropoxypyrimidine-2-carboxylate

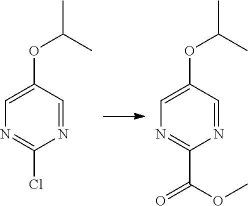

A stirred mixture of 2-chloro-5-isopropoxy-pyrimidine (40 g, 220.15 mmol) and Pd(dppf)₂Cl₂.DCM (10 g, 12.245 mmol) in DMF (200 mL)/MeOH (200 mL)/TEA (400.00 mL) in a 2 L steel bomb with an overhead mechanical stirrer was purged with carbon monoxide three times. The reaction mixture was heated to 120° C. with 120 psi of CO and retained at this temperature for one hour. Heating was turned off and the reaction mixture was allowed to cool to rt. Methanol and triethylamine were evaporated in vacuo. Water (1 L) was added, and the suspension filtered to remove catalyst residue. The cake was washed with water. The filtrate was extracted with DCM (3×700 mL). The combined organic layer was washed with brine, dried over sodium sulfate, filtered, and concentrated in vacuo. The resulting brown oil was purified by flash chromatography (Silica, 220 g, loaded in DCM, eluted with 25% ethyl acetate in hexanes) to give a greenish oil, which was triturated with hexanes and filtered. The cake was washed with hexanes and dried in vacuo to give methyl 5-isopropoxypyrimidine-2-carboxylate (30.1 g, 66%) as a beige solid. ESI-MS m/z calc. 196.0848, found 197.4 (M+1)⁺; Retention time: 3.15 minutes; LC method S.

### Step 7: 5-Isopropoxypyrimidine-2-carbaldehyde

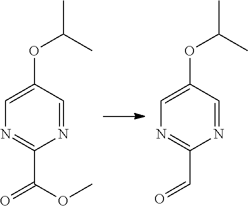

To a stirred solution of methyl 5-isopropoxypyrimidine-2-carboxylate (32.6 g, 157.85 mmol) in THF (1 L) was added DIBAL in toluene (240 mL of 1 M, 240.00 mmol) at −78° C. over 30 minutes and the reaction was stirred for 1 h. The reaction mixture was quenched with methanol (500 mL) and water (250 mL). The dry ice bath was removed, and the reaction mixture was allowed to warm to rt. The mixture was concentrated in vacuo to remove THF and methanol. DCM (2 L) was added, and the suspension was filtered. The organic layer from the filtrate was separated and the aqueous layer was extracted with DCM (2×1 L). The combined organic layer was dried over sodium sulfate, filtered, and concentrated in vacuo. The resulting yellow solid was triturated with hexanes and filtered. The cake was washed with hexanes and dried to give 5-isopropoxypyrimidine-2-carbaldehyde (23.3 g, 88%) as an off-white solid. ¹H NMR (500 MHz, DMSO-d₆) δ 9.89 (s, 1H), 8.75 (s, 2H), 4.99 (hept, J 6.0 Hz, 1H), 1.34 (d, J 6.0 Hz, 6H). ESI-MS m/z calc. 166.07423, found 167.2 (M+1)⁺; Retention time: 1.13 minutes; LC method W.

### Step 8: (2R)-2-[(5-Isopropoxypyrimidin-2-yl)methylamino]-5,5-dimethyl-hexan-1-ol

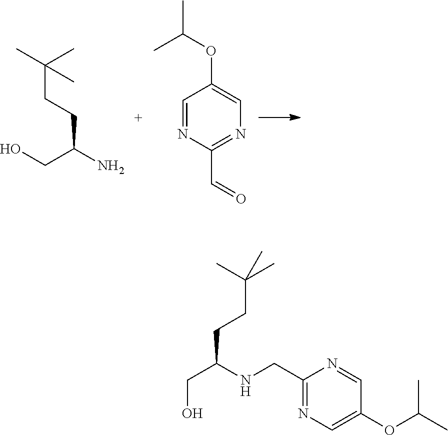

A 500 mL flask was charged under nitrogen with (2R)-2-amino-5,5-dimethyl-hexan-1-ol (hydrochloride salt) (3.75 g, 20.64 mmol) and 1,2-dichloroethane (80 mL). To the suspension was added DIEA (4 mL, 22.96 mmol) and the mixture was stirred for 8 minutes at room temperature. To the suspension was added 5-isopropoxypyrimidine-2-carbaldehyde (3.445 g, 20.73 mmol). After 8 min, acetic acid (1.3 mL, 22.86 mmol) was added, and the mixture was stirred at room temperature for 1.5 h. To the mixture was added sodium triacetoxyborohydride (5.67 g, 26.75 mmol) in three equal portions over 10 minutes and the mixture was continued to stir at room temperature for an additional 4 hours. The reaction mixture was cooled in an ice water bath and slowly quenched with aqueous NaOH (120 mL of 2 M, 240.0 mmol) and brine (50 mL). The reaction mixture was transferred to a separatory funnel, the layers were separated, and the aqueous layer was extracted with diethyl ether (2×30 mL). The combined organic phases were washed with brine (40 mL) and dried over sodium sulfate, filtered, and concentrated under reduced pressure. The crude material (6.52 g) was dissolved in DCM and purified by flash chromatography on silica gel (220 g column) using a gradient of methanol containing 10% v:v of concentrated aqueous ammonium hydroxide (0 to 15% over 40 min gradient) in dichloromethane to give (2R)-2-[(5-isopropoxypyrimidin-2-yl)methylamino]-5,5-dimethyl-hexan-1-ol (4.33 g, 71%) as a tan resin. ¹H NMR (400 MHz, Chloroform-d) δ 8.34 (s, 2H), 4.60 (hept, J 6.1 Hz, 1H), 4.11 (d, J 15.8 Hz, 1H), 4.02 (d, J 15.9 Hz, 1H), 3.65 (dd, J 11.0, 3.6 Hz, 1H), 3.39 (dd, J 11.0, 6.3 Hz, 1H), 2.69-2.60 (m, 1H), 1.55-1.40 (m, 2H), 1.38 (d, J 6.1 Hz, 6H), 1.29-1.15 (m, 2H), 0.88 (s, 9H). ESI-MS m/z calc. 295.22598, found 296.61 (M+1)⁺; Retention time: 1.05 minutes LC method A.

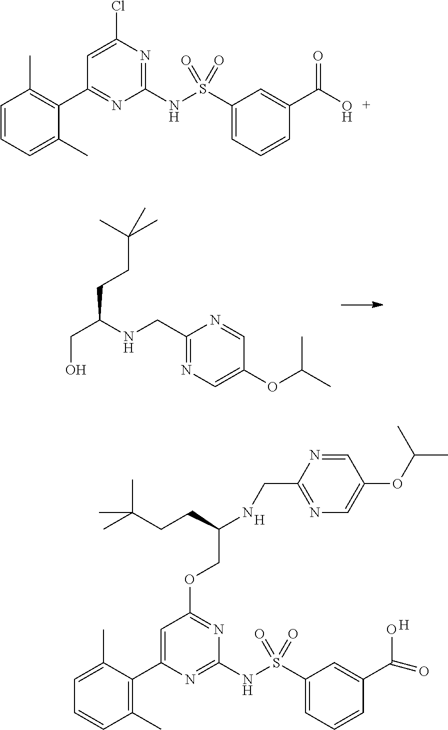

### Step 9: 3-[[4-(2,6-Dimethylphenyl)-6-[(2R)-2-[(5-isopropoxypyrimidin-2-yl)methylamino]-5,5-dimethyl-hexoxy]pyrimidin-2-yl]sulfamoyl]benzoic acid

A 500 mL flask was charged under nitrogen with (2R)-2-[(5-isopropoxypyrimidin-2-yl)methylamino]-5,5-dimethyl-hexan-1-ol (4.33 g, 14.66 mmol), 3-[[4-chloro-6-(2,6-dimethylphenyl)pyrimidin-2-yl]sulfamoyl]benzoic acid (6.15 g, 14.72 mmol) and anhydrous THF (100 mL). The mixture was stirred for about 10 min until all solids dissolved (internal temperature 19° C.). Sodium tert-butoxide (7 g, 72.84 mmol) was added quickly in two portions raising internal temperature to 33° C. and the reaction was stirred without external cooling for 2 h. The reaction mixture was cooled in an ice-water bath and then quenched by dropwise addition of an aqueous HCl (35 mL of 6 M, 210.0 mmol) solution at such a rate as to keep internal temperature below 15° C. (10 min addition time). Brine (40 mL), water (40 mL).and ethyl acetate (60 mL) were added, and the mixture was allowed to warm to room temperature under stirring. The reaction mixture was transferred to a separatory funnel. The layers were separated, and the aqueous layer was extracted once with EtOAc (50 mL). The combined organic phases were washed with water (50 mL) dried over sodium sulfate, filtered and the solution was concentrated to about 100 mL at which time the product started to crystallize out. The slurry was stirred in an ice bath for 30 min. The solid was filtered and washed with a small volume of cold ethyl acetate and dried by suction to give 3-[[4-(2,6-dimethylphenyl)-6-[(2R)-2-[(5-isopropoxypyrimidin-2-yl)methylamino]-5,5-dimethyl-hexoxy]pyrimidin-2-yl]sulfamoyl]benzoic acid (hydrochloride salt) (8.66 g, 82%) as a white solid. ¹H NMR (400 MHz, DMSO-d₆) δ 13.14 (broad s, 2H), 9.51 (br s, 2H), 8.60 (s, 2H), 8.46 (t, J=1.9 Hz, 1H), 8.15 (dd, J=7.9, 1.8 Hz, 2H), 7.69 (t, J 7.8 Hz, 1H), 7.26 (t, J 7.6 Hz, 1H), 7.13 (d, J 7.6 Hz, 2H), 6.34 (br s, 1H), 4.85 (hept, J 6.1 Hz, 1H), 4.65-4.39 (m, 4H), 3.68-3.55 (m, 1H), 2.13-1.92 (m, 6H), 1.87-1.75 (m, 1H), 1.73-1.57 (m, 1H), 1.31 (d, J 6.0 Hz, 6H), 1.25-1.14 (overlapped with residual EtOAc, m, 2H), 0.84 (s, 9H). ESI-MS m/z calc. 676.3043, found 677.68 (M+1)⁺; Retention time: 1.42 minutes; LC method A.

### Step 10: (11R)-11-(3,3-Dimethylbutyl)-6-(2,6-dimethylphenyl)-12-[(5-isopropoxypyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

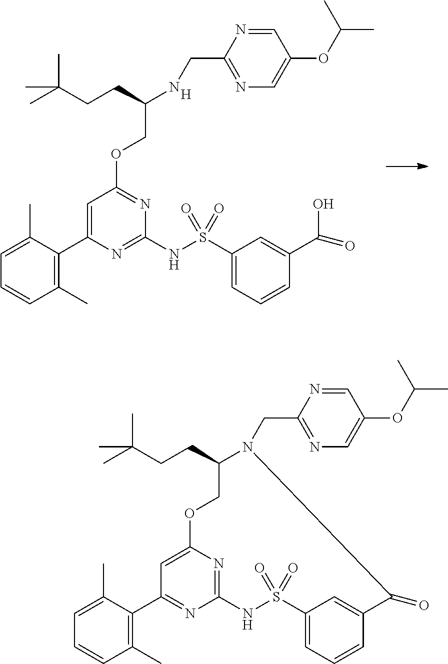

3-[[4-(2,6-Dimethylphenyl)-6-[(2R)-2-[(5-isopropoxypyrimidin-2-yl)methylamino]-5,5-dimethyl-hexoxy]pyrimidin-2-yl]sulfamoyl]benzoic acid (hydrochloride salt) (8.44 g, 11.83 mmol) was combined in a 1 L flask under nitrogen with anhydrous DMF (400 mL). Once all solids had dissolved, CDMT (3.55 g, 20.22 mmol) was added and the solution was stirred in an ice-water bath until internal temperature reached<2° C., at which time 4-Methyl-morpholine (7.3 mL, 66.40 mmol) was added through syringe over 4 minutes. The solution was stirred in the cooling bath that was allowed to warm to room temperature for 21 hours (at which time internal temperature had reached 22° C.). The reaction was concentrated under reduced pressure to about 100 mL (water bath at 50° C.). The mixture was poured into a separatory funnel with ethyl acetate (500 mL), brine (100 mL) and water (100 mL). The funnel was shaken, and the phases separated, and the aqueous layer was extracted with ethyl acetate (2×100 mL). The combined extracts were washed with brine (2×100 mL), dried over sodium sulfate and the solvents were concentrated to a crude amber oil that was diluted with a minimum volume of DCM and ethyl acetate. The solution was loaded on a 330 g silica gel column and purified by flash chromatography using a gradient of ethyl acetate (5 to 100% over 30 min) in hexanes. The pure fractions were combined, and the solvents evaporated to give 4.54 g of pure product. Mixed fractions containing about 13% of dimeric impurity were combined to give 2.0 g of material that was subjected to purification by reverse phase HPLC (C₁₈, 1-99% acetonitrile/5 mM aqueous HCl over 15 min). The pure fractions were combined, and acetonitrile was evaporated. The solid was extracted with ethyl acetate. The organic phase was dried over sodium sulfate, filtered, and combined with the 4.54 g of pure material. After evaporation of the solvents, 5.87 g of non-dry solid was isolated. This material was triturated in a mixture of EtOAc and hexanes (1:4, v:v, 100 mL) and the resulting white suspension was stirred for 30 min. The white solid was filtered and dried under vacuum at rt for 3 days to give (11R)-11-(3,3-dimethylbutyl)-6-(2,6-dimethylphenyl)-12-[(5-isopropoxypyrimidin-2-yl)methyl]-2,2-dioxo-9-oxa-2⁶-thia-3,5,12,19-tetrazatricyclo[12.3.1.14,8]nonadeca-1(18),4(19),5,7,14,16-hexaen-13-one (4.737 g, 60%) as a white solid. ¹H NMR (400 MHz, Chloroform-d) δ 10.08 (broad s, 1H), 8.87 (s, 1H), 8.37 (s, 2H), 8.21 (d, J 7.9 Hz, 1H), 7.82 (d, J 7.6 Hz, 1H), 7.69 (t, J 7.8 Hz, 1H), 7.18 (t, J 7.6 Hz, 1H), 7.00 (d, J 7.6 Hz, 2H), 6.14 (s, 1H), 5.51 (dd, J 11.3, 4.0 Hz, 1H), 5.37 (d, J 16.5 Hz, 1H), 4.60 (hept, J 6.1 Hz, 1H), 4.25 (d, J 16.6 Hz, 1H), 3.99 (s, 1H), 3.81 (t, J 11.4 Hz, 1H), 1.96 (s, 6H), 1.63-1.56 (overlapped with water m, 2H), 1.42-1.34 (m, 6H), 1.25-1.16 (m, 1H), 0.90-0.79 (m, 10H). ESI-MS m/z calc. 658.29376, found 659.27 (M+1)⁺; Retention time: 1.95 minutes; LC method A.

### Example 139: Preparation of Compound 1297

### Step 1: (2R)-2-Amino-3-cyclobutyl-propan-1-ol

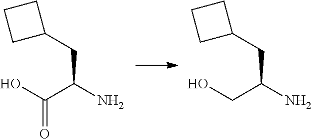 [SMILES: N[C@H](CC1CCC1)C(=O)O ; N[C@@H](CO)CC1CCC1]

Borane tetrahydrofuran complex in tetrahydrofuran (360 mL of 1 M, 360.00 mmol) was added dropwise over 20 minutes to a solution of (2R)-2-amino-3-cyclobutyl-propanoic acid (19.99 g, 139.61 mmol) in anhydrous THF (400 mL) at 0° C. under nitrogen and mechanical stirring. The reaction was stirred at 0° C. for 10 minutes and was then allowed to reach room temperature and stirred overnight at room temperature. The reaction was then cooled down to 0° C. and quenched by the dropwise addition of methanol (120 mL) over 20 minutes. The solution was concentrated under reduced pressure and taken up in ethyl acetate (400 mL) and washed with an aqueous solution of 1N sodium hydroxide (200 mL). The layers were separated, and the organic layer was concentrated under reduced pressure. The crude material was then taken up with an aqueous solution of 3 N hydrochloric acid (100) and co-evaporated with isopropanol (2×100 mL) and then MTBE (3×100 mL) to afford (2R)-2-amino-3-cyclobutyl-propan-1-ol (hydrochloride salt) (20.259 g, 70%) as a white solid. ESI-MS m/z calc. 129.11537, found 130.2 (M+1)⁺; Retention time: 1.7 minutes; LC method 1D.

### Step 2: (2R)-3-Cyclobutyl-2-[(5-isopropoxypyrimidin-2-yl)methylamino]propan-1-ol

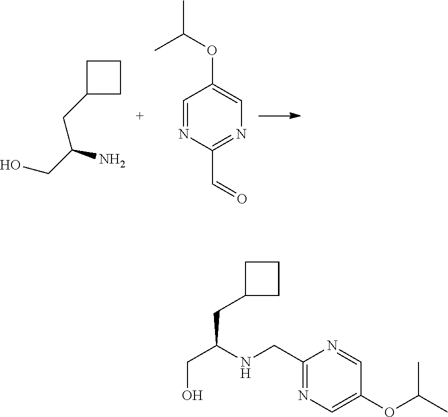

A 500 mL flask was charged under nitrogen with (2R)-2-amino-3-cyclobutyl-propan-1-ol (hydrochloride salt) (3.62 g, 21.85 mmol) and 1,2-dichloroethane (80 mL). To the suspension was added DIEA (4.2 mL, 24.11 mmol) and the mixture was stirred for 8 minutes at room temperature. To the suspension was added 5-isopropoxypyrimidine-2-carbaldehyde (3.64 g, 21.90 mmol). After most of the solids dissolved, acetic acid (1.4 mL, 24.62 mmol) was added, and the mixture was stirred at room temperature for 1.5 h. To the mixture was added sodium triacetoxyborohydride (6 g, 28.31 mmol) in three equal portions over 10 minutes and the mixture was continued to stir at room temperature for an additional 3 hours. The reaction mixture was cooled in an ice water bath and slowly quenched with aqueous NaOH (120 mL of 2 M, 240.0 mmol) and brine (50 mL). The reaction mixture was transferred to a separatory funnel, the layers were separated, and the aqueous layer was extracted with diethyl ether (3×30 mL). The combined organic phases were washed with brine (40 mL) and dried over sodium sulfate, filtered, and concentrated under reduced pressure. The crude material was dissolved in DCM and purified by flash chromatography on silica gel (220 g column) using a gradient of methanol (containing 10% v:v of concentrated aqueous ammonium hydroxide, 0 to 15% over 40 min gradient) in dichloromethane to give (2R)-3-cyclobutyl-2-[(5-isopropoxypyrimidin-2-yl)methylamino]propan-1-ol (4.31 g, 71%) as a tan resin. ¹H NMR (400 MHz, Chloroform-d) δ 8.34 (s, 2H), 4.60 (hept, J 6.1 Hz, 1H), 4.13-3.95 (m, 2H), 3.60 (dd, J 10.9, 3.6 Hz, 1H), 3.32 (dd, J=10.9, 6.4 Hz, 1H), 2.68-2.58 (m, 1H), 2.39 (hept, J=7.9 Hz, 1H), 2.11-1.98 (m, 2H), 1.92-1.74 (m, 2H), 1.69-1.50 (m, 4H), 1.38 (d, J=6.1 Hz, 6H). ESI-MS m/z calc. 279.19467, found 280.6 (M+1)⁺; Retention time: 0.92 minutes; LC method A.

### Step 3: 3-[[4-[(2R)-3-Cyclobutyl-2-[(5-isopropoxypyrimidin-2-yl)methylamino]propoxy]-6-(2,6-dimethylphenyl)pyrimidin-2-yl]sulfamoyl]benzoic acid

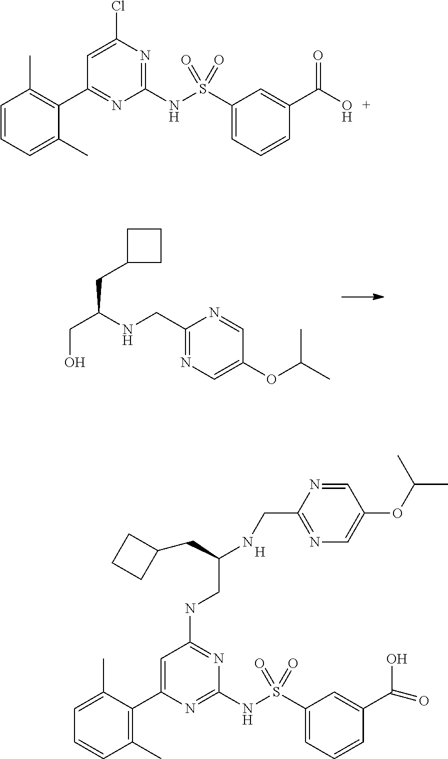

A 500 mL flask was charged under nitrogen with (2R)-3-cyclobutyl-2-[(5-isopropoxypyrimidin-2-yl)methylamino]propan-1-ol (4.31 g, 15.43 mmol), 3-[[4-chloro-6-(2,6-dimethylphenyl)pyrimidin-2-yl]sulfamoyl]benzoic acid (6.45 g, 15.44 mmol) and anhydrous THF (100 mL). The mixture was stirred for about 10 min until all solids dissolved. Sodium tert-butoxide (7.34 g, 76.38 mmol) was added in two portions raising internal temperature to 36° C. and the reaction was stirred without external cooling for 1 h. The reaction mixture was cooled in an ice-water bath and then quenched by dropwise addition of an aqueous HCl (32 mL of 6 M, 192.0 mmol) solution at such a rate as to keep internal temperature below 20° C. (10 min addition time). Brine (40 mL), 1N aqueous HCl (40 mL) and ethyl acetate (60 mL) were added, and the mixture was allowed to warm to room temperature under stirring. The resulting emulsion dissipated upon adding additional water (40 mL). The reaction mixture was transferred to a separatory funnel. The layers were separated, and the aqueous layer was extracted once with EtOAc (50 mL). The combined organic phases were washed with water (50 mL) dried over sodium sulfate, filtered, and concentrated to give a cream foamy crude product (11.25 g). The material was dissolved in DCM containing a bit of methanol and purified by flash chromatography on silica gel (330 g column) using a gradient of methanol (0 to 15% over 30 min) in dichloromethane. After evaporation, the residue was triturated with DCM/hexanes and the solvents were evaporated. The operation was repeated twice. After drying under vacuum, 3-[[4-[(2R)-3-cyclobutyl-2-[(5-isopropoxypyrimidin-2-yl)methylamino]propoxy]-6-(2,6-dimethylphenyl)pyrimidin-2-yl]sulfamoyl]benzoic acid (hydrochloride salt) (9.53 g, 88%) was isolated as an off-white solid. ¹H NMR (400 MHz, DMSO-d₆) δ 13.87-9.32 (broad m, 4H), 8.57 (s, 2H), 8.46 (s, 1H), 8.14 (dd, J 12.3, 7.8 Hz, 2H), 7.70 (t, J 7.8 Hz, 1H), 7.26 (t, J 7.6 Hz, 1H), 7.13 (d, J 7.6 Hz, 2H), 6.31 (s, 1H), 4.83 (hept, J 6.0 Hz, 1H), 4.50-4.30 (m, 3H), 4.23 (dd, J 12.4, 5.4 Hz, 1H), 3.43 (s, 1H), 2.33 (hept, J 7.6 Hz, 1H), 2.12-1.87 (m, 8H), 1.85-1.70 (m, 3H), 1.70-1.49 (m, 2H), 1.35-1.17 (m, 7H). ESI-MS m/z calc. 660.273, found 661.62 (M+1)⁺; Retention time: 1.34 minutes; LC method A.

### Step 4: (11R)-11-(Cyclobutylmethyl)-6-(2,6-dimethylphenyl)-12-[(5-isopropoxypyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

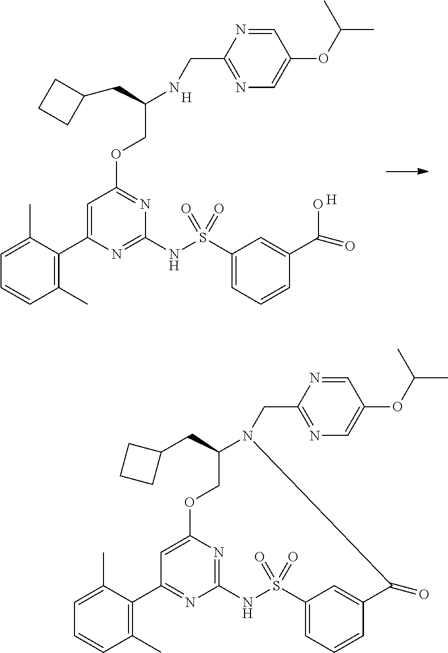

3-[[4-[(2R)-3-Cyclobutyl-2-[(5-isopropoxypyrimidin-2-yl)methylamino]propoxy]-6-(2,6-dimethylphenyl)pyrimidin-2-yl]sulfamoyl]benzoic acid (hydrochloride salt) (9.53 g, 13.67 mmol) was combined in a 1 L flask under nitrogen with anhydrous DMF (450 mL). Once all solids had dissolved, CDMT (4.1 g, 23.35 mmol) was added and the solution was stirred in an ice-water bath until internal temperature reached 2° C., at which time 4-Methyl-morpholine (8.4 mL, 76.40 mmol) was added through syringe over 3 minutes. The solution was stirred in the cooling bath that was allowed to slowly warm up to room temperature. After 15 hours under stirring, the reaction was concentrated under reduced pressure to about half of its volume (water bath at 50° C.). The mixture was poured into a separatory funnel with ethyl acetate (500 mL), brine (100 mL) and water (100 mL). The funnel was shaken, and the phases separated and the aqueous layer was extracted with ethyl acetate (2×100 mL). The combined extracts were washed with brine (100 mL), dried over sodium sulfate and the solvents were concentrated to a crude oil (17.0 g). The material was diluted with a minimum volume of DCM containing a bit of ethyl acetate and purified by flash chromatography on silica gel (330 g column) using a gradient of ethyl acetate (5 to 100% over 30 min) in hexanes. The pure fractions were combined, and the solvents evaporated to give 5.4 g of a white solid. This material was slurried in a mixture of EtOAc and hexanes (1:3, v:v, 50 mL), filtered and dried in a vacuum oven at 50° C. for 3 days to give (11R)-11-(cyclobutylmethyl)-6-(2,6-dimethylphenyl)-12-[(5-isopropoxypyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (5.237 g, 58%) as a white microcrystalline solid. ¹H NMR (400 MHz, Chloroform-d) δ 9.97 (s, 1H), 8.87 (d, J=1.8 Hz, 1H), 8.39-8.32 (m, 2H), 8.10 (d, J 7.9 Hz, 1H), 7.81 (dt, J 7.6, 1.4 Hz, 1H), 7.64 (t, J 7.8 Hz, 1H), 7.19 (t, J 7.6 Hz, 1H), 7.02 (d, J 7.6 Hz, 2H), 6.16 (s, 1H), 5.49 (dd, J 11.2, 4.1 Hz, 1H), 5.36 (d, J 16.6 Hz, 1H), 4.60 (hept, J 6.0 Hz, 1H), 4.26 (d, J 16.6 Hz, 1H), 3.98 (tt, J 11.1, 3.9 Hz, 1H), 3.78 (t, J 11.4 Hz, 1H), 2.17 (hept, J 7.8 Hz, 1H), 1.98 (s, 6H), 1.87-1.74 (m, 2H), 1.73-1.53 (m, 5H), 1.40-1.33 (m, 6H), 1.20-1.06 (m, 1H). ESI-MS m/z calc. 642.26245, found 643.62 (M+1)⁺; Retention time: 1.93 minutes; LC method A.

### Example 140: Preparation of Compound 1298

### Step 1: 3-[[4-[(2R)-2-Amino-4,4-dimethyl-pentoxy]-6-(2,6-dimethylphenyl)pyrimidin-2-yl]-(methoxymethyl)sulfamoyl]benzoic acid

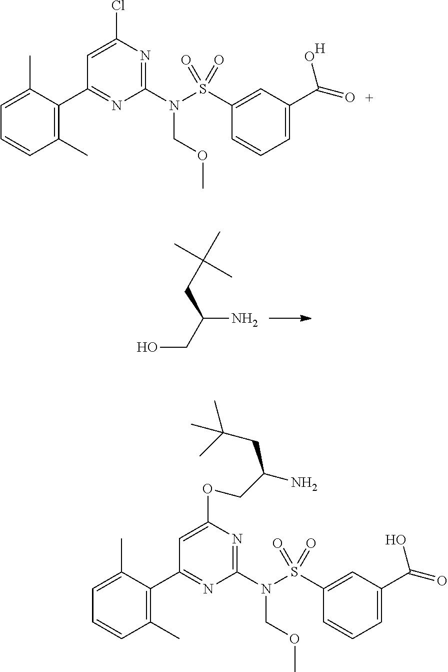

In a 100 mL flask, 3-[[4-chloro-6-(2,6-dimethylphenyl)pyrimidin-2-yl]-(methoxymethyl)sulfamoyl]benzoic acid (3.25 g, 6.684 mmol), (2R)-2-amino-4,4-dimethyl-pentan-1-ol (hydrochloride salt) (1.13 g, 6.739 mmol) and anhydrous THF (9 mL) were combined under nitrogen to give a suspension. Sodium tert-butoxide (2.71 g, 28.20 mmol) was added (important exotherm observed and partial dissolution of solids) and the mixture was stirred at room temperature for 1 h. The reaction was diluted with ethyl acetate (50 mL) and washed with a mixture of aqueous 1M HCl (30 mL) and brine (30 mL). After separation, the aqueous phase was extracted with EtOAc (30 mL). The combined organics were further washed with brine, dried over sodium sulfate and filtered. The product started to precipitate from the EtOAc solution. After concentration, the solid was slurried in a 1:1 (v:v) mixture of EtOAc and hexanes (150 mL). The solid was filtered and dried to give 3-[[4-[(2R)-2-amino-4,4-dimethyl-pentoxy]-6-(2,6-dimethylphenyl)pyrimidin-2-yl]-(methoxymethyl)sulfamoyl]benzoic acid (hydrochloride salt) (2.62 g, 66%) as a white solid. ESI-MS m/z calc. 556.23553, found 557.19 (M+1)⁺; Retention time: 1.4 minutes. ¹H NMR (400 MHz, DMSO-d₆) δ 13.41 (broad s, 1H), 8.44 (t, J 1.8 Hz, 1H), 8.27-8.05 (m, 5H), 7.61 (t, J 7.9 Hz, 1H), 7.22 (t, J 7.6 Hz, 1H), 7.09 (d, J 7.6 Hz, 2H), 6.52 (s, 1H), 5.67 (s, 2H), 4.33 (dd, J 11.7, 3.3 Hz, 1H), 4.15 (dd, J 11.7, 7.1 Hz, 1H), 3.55 (s, 1H), 3.32 (s, 3H), 1.85 (s, 6H), 1.66-1.45 (m, 2H), 0.94 (s, 9H). LC method A.

### Step 2: (11R)-6-(2,6-Dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

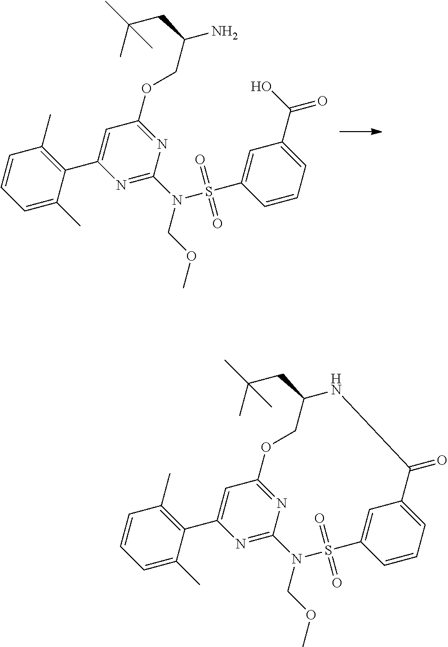 [SMILES: COCN(c1nc(OC[C@H](N)CC(C)(C)C)cc(-c2c(C)cccc2C)n1)S(=O)(=O)c1cccc(C(=O)O)c1 ; COCN1c2nc(cc(-c3c(C)cccc3C)n2)OC[C@@H](CC(C)(C)C)NC(=O)c2cccc(c2)S1(=O)=O]

To a solution of HATU (11.8 g, 31.034 mmol) and DIPEA (12.614 g, 17 mL, 97.599 mmol) in DMF (120 mL) was added a solution of 3-[[4-[(2R)-2-amino-4,4-dimethyl-pentoxy]-6-(2,6-dimethylphenyl)pyrimidin-2-yl]-(methoxymethyl)sulfamoyl]benzoic acid (hydrochloride salt) (14.8 g, 24.753 mmol) in DMF (110 mL). The reaction was stirred at room temperature for 20 min and poured into 370 ml of water. The resulting solid was filtered. The crude material was suspended in DMF (60 ml) and MeCN (50 mL) was added. The mixture was sonicated for 30 min and filtered. The solid was dissolved in DCM (100 mL) and the resulting organic layer was washed first with water (20 mL), then with a 1:1 v/v mix of water and brine (3×50 mL) and brine (2×20 mL). Afforded (11R)-6-(2,6-dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (5.3 g, 39%) as an off-white solid. ¹H NMR (400 MHz, DMSO-d₆) δ 8.72 (s, 1H), 8.14 (d, J=7.1 Hz, 1H), 7.97 (d, J=9.8 Hz, 1H), 7.88-7.76 (m, 2H), 7.22 (t, J 8.1 Hz, 1H), 7.12 (s, 1H), 7.10 (s, 1H), 6.72 (s, 1H), 5.70 (d, J 11.2 Hz, 1H), 5.53 (d, J 11.2 Hz, 1H), 5.04 (dd, J 11.2, 4.2 Hz, 1H), 3.87 (t, J 11.4 Hz, 1H), 3.57-3.45 (m, 1H), 2.88 (s, 3H), 1.96 (br. s., 6H), 1.57-1.41 (m, 2H), 0.51 (s, 9H). ESI-MS m/z calc. 538.225, found 539.2 (M+1)⁺; Retention time: 4.71 minutes; LC method Y.

### Step 3: (11R)-6-(2,6-Dimethylphenyl)-11-(2,2-dimethylpropyl)-3-(methoxymethyl)-9-oxa-2λ⁶-thia-3,5,12,19-tetrazatricyclo[12.3.1.14,8]nonadeca-1(18),4(19),5,7,14,16-hexaene 2,2-dioxide

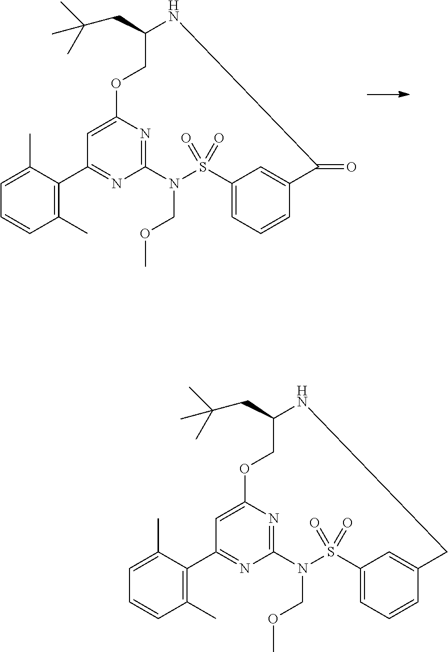 [SMILES: COCN1c2nc(cc(-c3c(C)cccc3C)n2)OC[C@@H](CC(C)(C)C)NC(=O)c2cccc(c2)S1(=O)=O ; COCN1c2nc(cc(-c3c(C)cccc3C)n2)OC[C@@H](CC(C)(C)C)NCc2cccc(c2)S1(=O)=O]

In a reaction vial, (11R)-6-(2,6-dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (150 mg, 0.2785 mmol) was dissolved in THF (2 mL) along with borane tetrahydrofuran (1.39 mL of 1 M, 1.390 mmol). The reaction mixture was refluxed for 2 h. then quenched with MeOH and evaporated to dryness. The crude material was purified by column chromatography on silica using 20-80% ethyl acetate/hexanes gradient. The product was isolated as a clear oil. (11R)-6-(2,6-dimethylphenyl)-11-(2,2-dimethylpropyl)-3-(methoxymethyl)-9-oxa-2λ⁶-thia-3,5,12,19-tetrazatricyclo[12.3.1.14,8]nonadeca-1(18),4(19),5,7,14,16-hexaene 2,2-dioxide (110.2 mg, 68%). ¹H NMR (400 MHz, DMSO-d₆) δ 8.57 (s, 1H), 7.88-7.81 (m, 1H), 7.63-7.57 (m, 2H), 7.23-7.17 (m, 1H), 7.09 (dd, J 7.7, 3.4 Hz, 2H), 4.35 (t, J 5.2 Hz, 2H), 4.10-3.93 (m, 2H), 3.38 (q, J 6.3, 5.1 Hz, 3H), 2.99 (s, 2H), 1.95 (s, 6H), 0.87 (t, J 7.3 Hz, 5H), 0.57 (d, J 12.6 Hz, 9H). ESI-MS m/z calc. 524.2457, found 525.3 (M+1)⁺; Retention time: 1.53 minutes. LC method A.

### Step 4: (11R)-6-(2,6-Dimethylphenyl)-11-(2,2-dimethylpropyl)-12-[(5-isopropoxypyrimidin-2-yl)methyl]-9-oxa-2λ⁶-thia-3,5,12,19-tetrazatricyclo[12.3.1.14,8]nonadeca-1(18),4(19),5,7,14,16-hexaene 2,2-dioxide

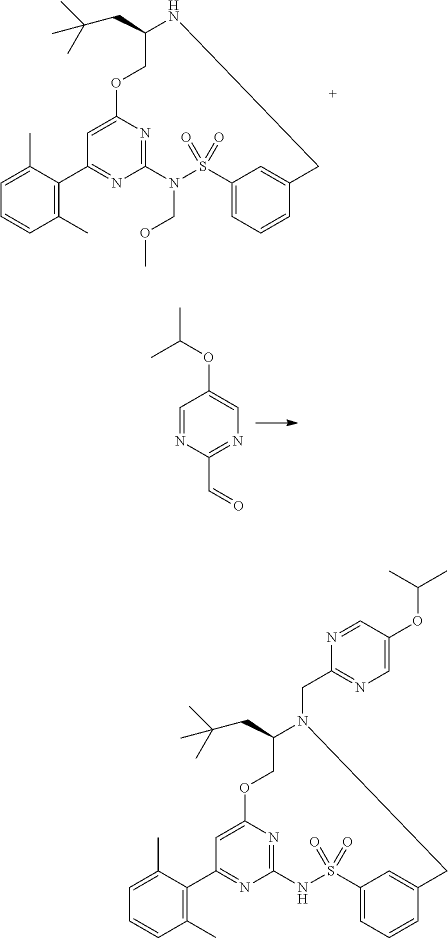 [SMILES: COCN1c2nc(cc(-c3c(C)cccc3C)n2)OC[C@@H](CC(C)(C)C)NCc2cccc(c2)S1(=O)=O ; CC(C)Oc1cnc(C=O)nc1 ; Cc1cccc(C)c1-c1cc2nc(n1)NS(=O)(=O)c1cccc(c1)CN(Cc1ncc(OC(C)C)cn1)[C@H](CC(C)(C)C)CO2]

In a reaction vial, (11R)-6-(2,6-dimethylphenyl)-11-(2,2-dimethylpropyl)-3-(methoxymethyl)-9-oxa-2λ⁶-thia-3,5,12,19-tetrazatricyclo[12.3.1.14,8]nonadeca-1(18),4(19),5,7,14,16-hexaene 2,2-dioxide (50 mg, 0.08577 mmol) was mixed with 5-isopropoxypyrimidine-2-carbaldehyde (14.3 mg, 0.08605 mmol) and acetic acid (4.9 μL, 0.08617 mmol) in DCE (300 μL). To the reaction mixture, sodium triacetoxyborohydride (26 mg, 0.1227 mmol) was added and the reaction was allowed to stir at rt for 3 h. The reaction was quenched with 1N NaOH and extracted with ether. The organic layer was washed with saturated NaCl soln., isolated, dried over anhydrous magnesium sulfate, filtered, and evaporated to dryness. The isolated crude intermediate was used for the next step with further purification. (11R)-6-(2,6-Dimethylphenyl)-11-(2,2-dimethylpropyl)-12-[(5-isopropoxypyrimidin-2-yl)methyl]-3-(methoxymethyl)-9-oxa-2λ⁶-thia-3,5,12,19-tetrazatricyclo[12.3.1.14,8]nonadeca-1(18),4(19),5,7,14,16-hexaene 2,2-dioxide (53.1 mg, 78%), was dissolved in DCM (500 μL) along with trifluoroacetic acid (1 mL, 12.98 mmol) and stirred at rt for 3 h. The reaction mixture was evaporated to dryness and the crude material was purified by prep. HPLC using 30-99% ACN/H₂O gradient with HCl modifier to give (11R)-6-(2,6-dimethylphenyl)-11-(2,2-dimethylpropyl)-12-[(5-isopropoxypyrimidin-2-yl)methyl]-9-oxa-2?⁶-thia-3,5,12,19-tetrazatricyclo[12.3.1.14,8]nonadeca-1(18),4(19),5,7,14,16-hexaene 2,2-dioxide (hydrochloride salt) (24.7 mg, 43%). ¹H NMR (400 MHz, DMSO-d₆) δ 8.54 (s, 3H), 7.66 (s, 1H), 7.42 (d, J 32.4 Hz, 2H), 7.23 (s, 1H), 7.14-7.04 (m, 2H), 6.27 (s, 1H), 5.19 (d, J 9.7 Hz, 1H), 4.79 (p, J 6.0 Hz, 1H), 4.18-4.04 (m, 2H), 3.99 (d, J 14.1 Hz, 1H), 3.84 (dd, J 14.6, 6.8 Hz, 2H), 2.81-2.69 (m, 1H), 2.21-1.85 (m, 6H), 1.80 (dd, J 14.7, 4.1 Hz, 1H), 1.30 (d, J 6.1 Hz, 6H), 0.91-0.79 (m, 2H), 0.57 (s, 9H). ESI-MS m/z calc. 630.2988, found 631.4 (M+1)⁺; Retention time: 2.05 minutes; LC method A.

### Example 141: Preparation of Compound 1299

### Step 1: 2-Chloro-5-(cyclobutoxy)-3-fluoro-pyridine

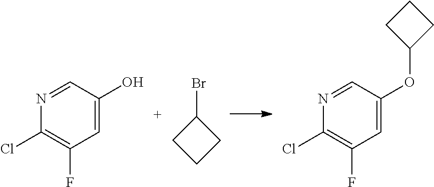 [SMILES: Oc1cnc(Cl)c(F)c1 ; BrC1CCC1 ; Fc1cc(OC2CCC2)cnc1Cl]

A mixture of 6-chloro-5-fluoro-pyridin-3-ol (10 g, 66.425 mmol), bromocyclobutane (13 g, 96.295 mmol) and potassium carbonate (25 g, 180.89 mmol) in DMF (100.00 mL) was stirred at 100° C. for 4 h. The reaction mixture was allowed to cool to rt, water (1 L) was added, extracted with DCM (3×500 mL). The combined organic layer was washed with brine (4×250 mL), dried over sodium sulfate, filtered and concentrated in vacuo. The resulting yellow oil was purified by flash chromatography (Silica, 120 g, loaded in DCM, eluted with 15% ethyl acetate in hexanes). The desired product fractions were combined and concentrated in vacuo to give 2-chloro-5-(cyclobutoxy)-3-fluoro-pyridine (12.74 g, 93%) as a white solid. ESI-MS m/z calc. 201.03568, found 202.2 (M+1)⁺; Retention time: 6.07 minutes; LC method S.

### Step 2: Methyl 5-(cyclobutoxy)-3-fluoro-pyridine-2-carboxylate

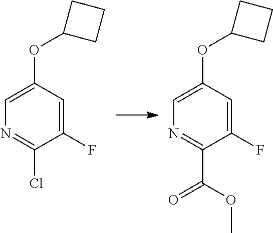 [SMILES: Fc1cc(OC2CCC2)cnc1Cl ; COC(=O)c1ncc(OC2CCC2)cc1F]

A mixture of 2-chloro-5-(cyclobutoxy)-3-fluoro-pyridine (12.7 g, 61.728 mmol) and Pd(dppf)₂Cl₂CH₂Cl₂ (3.0 g, 3.6736 mmol) in a mixture of DMF (130 mL), Et₃N (130 mL) and MeOH (200 mL) in a 2 L steel bomb with an overhead mechanical stirrer was purged with carbon monoxide three times. The reaction mixture was heated to 100° C. with 120 psi of CO over one hour and retained at this temperature for one hour. Heating was turned off and the reaction mixture was allowed to cool to rt. Methanol and triethylamine were evaporated in vacuo. Water (500 mL) was added and filtered to remove the catalyst residue. The cake was washed with water. The filtrate was extracted with DCM (3×500 mL). The combined organic layer was washed with brine, dried over sodium sulfate, filtered, and concentrated in vacuo. The resulting brown oil was purified by flash chromatography (Silica, 120 g, loaded in DCM, eluted with 15% ethyl acetate in hexanes) to give methyl 5-(cyclobutoxy)-3-fluoro-pyridine-2-carboxylate (10.79 g, 74%) as a pale-yellow solid. ESI-MS m/z calc. 225.08012, found 226.4 (M+1)⁺; Retention time: 5.06 minutes; LC method S.

### Step 3: 5-(Cyclobutoxy)-3-fluoro-pyridine-2-carbaldehyde

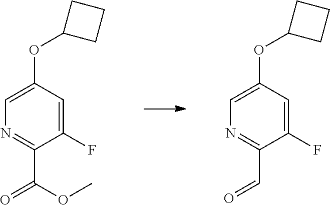 [SMILES: COC(=O)c1ncc(OC2CCC2)cc1F ; O=Cc1ncc(OC2CCC2)cc1F]

To a stirred solution of methyl 5-(cyclobutoxy)-3-fluoro-pyridine-2-carboxylate (10.9 g, 45.978 mmol) in THF (350 mL) was added DIBAL in toluene (70 mL of 1 M, 70.000 mmol) at −78° C. over 15 minutes and stirred for 2 h. The reaction mixture was quenched with methanol (150 mL) and water (100 mL). The dry ice bath was removed, and the reaction mixture was allowed to warm to rt. The mixture was concentrated in vacuo to remove THF and methanol. DCM (1 L) was added and filtered. The organic layer from the filtrate was separated and the aqueous layer was extracted with DCM (2×500 mL). The combined organic layer was dried over sodium sulfate, filtered, and concentrated in vacuo. The resulting yellow solid was purified by flash chromatography (Silica, 120 g, loaded in DCM, eluted with 20% ethyl acetate in hexanes). The desired product fractions were combined and concentrated in vacuo to give 5-(cyclobutoxy)-3-fluoro-pyridine-2-carbaldehyde (6.29 g, 70%) as a clear oil, which solidified upon freezing. ¹H NMR (500 MHz, DMSO-d₆) δ 9.93 (s, 1H), 8.30 (dd, J 2.4, 1.0 Hz, 1H), 7.44 (dd, J 12.6, 2.3 Hz, 1H), 5.00-4.83 (m, 1H), 2.58-2.44 (m, 2H), 2.17-2.02 (m, 2H), 1.82 (qt, J 10.2, 2.8 Hz, 1H), 1.72-1.55 (m, 1H). ESI-MS m/z calc. 195.06955, found 196.0 (M+1)⁺; Retention time: 1.86 minutes; LC method W.

### Step 4: 3-[[4-[(2R)-2-[[5-(Cyclobutoxy)-3-fluoro-2-pyridyl]methylamino]-4,4-dimethyl-pentoxy]-6-(2,6-dimethylphenyl)pyrimidin-2-yl]sulfamoyl]benzoic acid

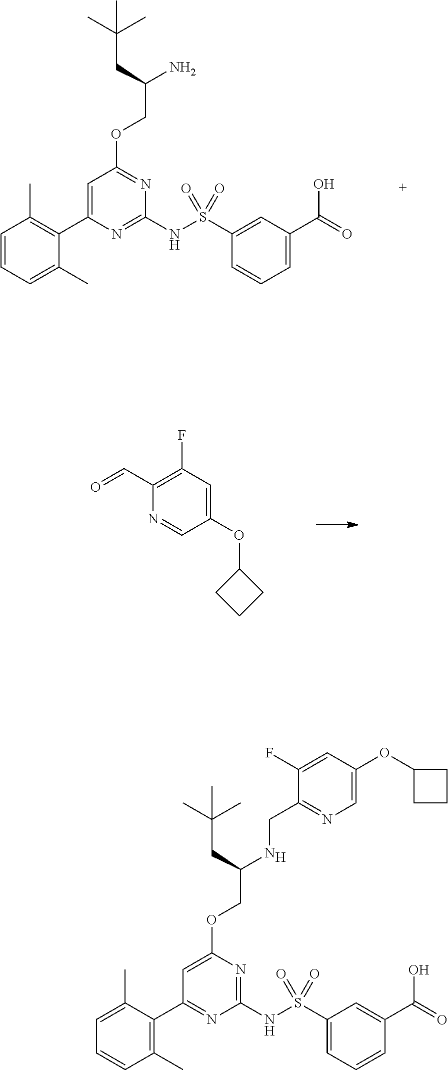

To a suspension of 3-[[4-[(2R)-2-amino-4,4-dimethyl-pentoxy]-6-(2,6-dimethylphenyl)pyrimidin-2-yl]sulfamoyl]benzoic acid (hydrochloride salt) (60.1 mg, 0.1095 mmol) in dichloromethane (1.0 mL) was added 5-(cyclobutoxy)-3-fluoro-pyridine-2-carbaldehyde (23.5 mg, 0.1204 mmol). The reaction mixture was stirred at room temperature for 15 minutes, then sodium triacetoxyborohydride (23.5 mg, 0.1109 mmol) was added. After stirring at room temperature for 10 minutes, additional sodium triacetoxyborohydride (11.5 mg, 0.05426 mmol) was added. The reaction was stirred at room temperature for another 10 minutes. The reaction was partitioned between ethyl acetate and 1N HCl, then the reaction mixture was extracted with ethyl acetate (3×) and the organic layer was washed with brine. The organic layer was isolated, dried over anhydrous magnesium sulfate, filtered, and evaporated to dryness. The crude material was filtered and purified (reverse-phase HPLC, C₁₈, acetonitrile in water with added HCl, 1-99% gradient) to give pure fractions, which were frozen and lyophilized to give 3-[[4-[(2R)-2-[[5-(cyclobutoxy)-3-fluoro-2-pyridyl]methylamino]-4,4-dimethyl-pentoxy]-6-(2,6-dimethylphenyl)pyrimidin-2-yl]sulfamoyl]benzoic acid (hydrochloride salt) (54.0 mg, 67%) as a white solid. ESI-MS m/z calc. 691.284, found 692.6 (M+1)⁺; Retention time: 1.46 minutes; LC method A. Step 5: (11R)-12-[[5-(Cyclobutoxy)-3-fluoro-2-pyridyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1299)

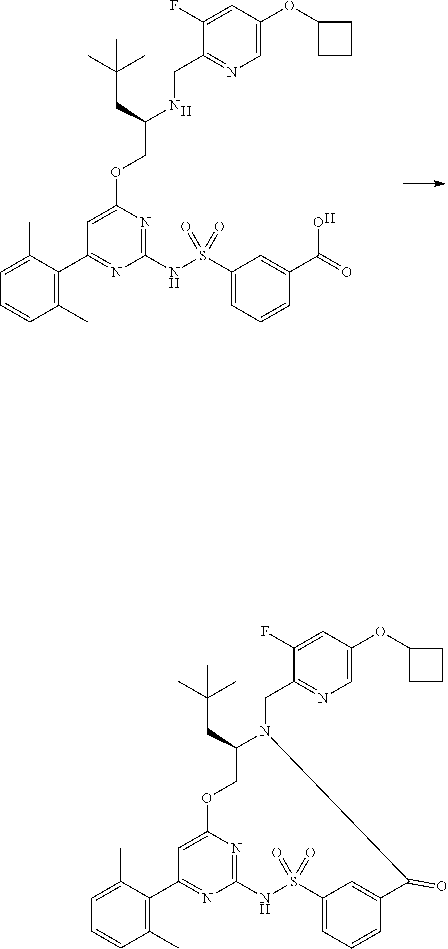

In a reaction vial, 3-[[4-[(2R)-2-[[5-(cyclobutoxy)-3-fluoro-2-pyridyl]methylamino]-4,4-dimethyl-pentoxy]-6-(2,6-dimethylphenyl)pyrimidin-2-yl]sulfamoyl]benzoic acid (hydrochloride salt) (54 mg, 0.07415 mmol) and 2-chloro-4,6-dimethoxy-1,3,5-triazine (17.4 mg, 0.09910 mmol) were dissolved in DMF (2.5 mL) and cooled to 0° C. After stirring for 5 minutes, 4-methylmorpholine (18.0 μL, 0.1637 mmol) was added. The reaction was stirred at 0° C. for 1 hour, then warmed to rt and stirred for an additional 3.5 hours. Additional portions of 2-chloro-4,6-dimethoxy-1,3,5-triazine (23.1 mg, 0.1316 mmol) and 4-methylmorpholine (20.4 μL, 0.1856 mmol) were added and stirring continued for an additional 16 hours. The reaction mixture was filtered and purified (reverse-phase HPLC, C₁₈, acetonitrile in water with added HCl, 1-99% gradient) to give pure fractions which were frozen and lyophilized to give (11R)-12-[[5-(cyclobutoxy)-3-fluoro-2-pyridyl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (22.3 mg, 44%) as a white solid. ESI-MS m/z calc. 673.27344, found 674.5 (M+1)⁺; Retention time: 2.11 minutes. ¹H NMR (400 MHz, DMSO) δ 13.07 (s, 1H), 8.59 (s, 1H), 8.07 (d, J=2.4 Hz, 1H), 7.94 (s, 1H), 7.66 (s, 2H), 7.34-7.22 (m, 2H), 7.13 (d, J=7.2 Hz, 2H), 6.43 (s, 1H), 5.35 (d, J 8.2 Hz, 1H), 4.90 (d, J 15.5 Hz, 1H), 4.82 (p, J 7.1 Hz, 1H), 4.44 (d, J 15.8 Hz, 1H), 4.24 (t, J 11.2 Hz, 1H), 4.07-3.98 (m, 1H), 2.47-2.42 (m, 2H), 2.20-1.91 (m, 8H), 1.85-1.74 (m, 2H), 1.70-1.56 (m, 1H), 1.41 (d, J 15.0 Hz, 1H), 0.56 (s, 9H); LC method A.

### Example 142: Preparation of Compound 1300

### Step 1: 3-[[4-[(2R)-2-[(6-Chloro-3-fluoro-2-pyridyl)methylamino]-4,4-dimethyl-pentoxy]-6-(2,6-dimethylphenyl)pyrimidin-2-yl]sulfamoyl]benzoic acid

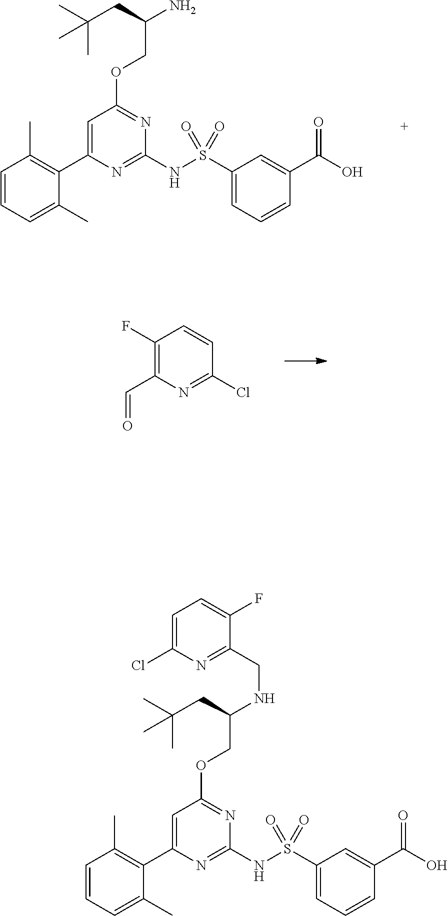 [SMILES: Cc1cccc(C)c1-c1cc(OC[C@H](N)CC(C)(C)C)nc(NS(=O)(=O)c2cccc(C(=O)O)c2)n1 ; O=Cc1nc(Cl)ccc1F ; Cc1cccc(C)c1-c1cc(OC[C@@H](CC(C)(C)C)NCc2nc(Cl)ccc2F)nc(NS(=O)(=O)c2cccc(C(=O)O)c2)n1]

To a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (625 mg, 1.138 mmol) and 6-chloro-3-fluoro-pyridine-2-carbaldehyde (272 mg, 1.705 mmol) in anhydrous DCM (6.5 mL) was added sodium triacetoxyborohydride (724 mg, 3.416 mmol). The reaction mixture was stirred at rt for 2 h. Then the mixture was poured over cold hydrochloric acid (10 mL of 1.0 M) and extracted with ethyl acetate. The combined organics were washed with brine, dried over sodium sulfate, filtered, and concentrated. 1:1 Ethyl acetate and hexanes was added to the residue and sonicated for 1 min. The resultant precipitate was collected by filtration and dried to give a light brown solid of 3-[[4-[(2R)-2-[(6-chloro-3-fluoro-2-pyridyl)methylamino]-4,4-dimethyl-pentoxy]-6-(2,6-dimethylphenyl)pyrimidin-2-yl]sulfamoyl]benzoic acid (hydrochloride salt) (481 mg, 30%) ESI-MS m/z calc. 655.2031, found 656.5 (M+1)⁺; Retention time: 0.51 minutes; LC method D.

### Step 2: (11R)-12-[(6-Chloro-3-fluoro-2-pyridyl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

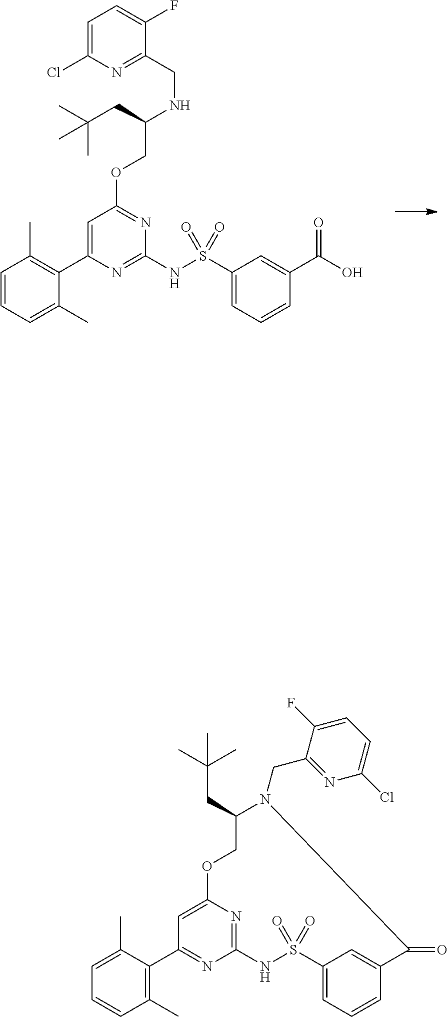 [SMILES: Cc1cccc(C)c1-c1cc(OC[C@@H](CC(C)(C)C)NCc2nc(Cl)ccc2F)nc(NS(=O)(=O)c2cccc(C(=O)O)c2)n1 ; Cc1cccc(C)c1-c1cc2nc(n1)NS(=O)(=O)c1cccc(c1)C(=O)N(Cc1nc(Cl)ccc1F)[C@H](CC(C)(C)C)CO2]

To a solution of 3-[[4-[(2R)-2-[(6-chloro-3-fluoro-2-pyridyl)methylamino]-4,4-dimethyl-pentoxy]-6-(2,6-dimethylphenyl)pyrimidin-2-yl]sulfamoyl]benzoic acid (hydrochloride salt) (967 mg, 1.396 mmol) in DMF (43.5 mL) was added 2-chloro-4,6-dimethoxy-1,3,5-triazine (245 mg, 1.395 mmol) and cooled to 0° C. To the reaction, was added 4-methylmorpholine (424 mg, 4.192 mmol). The reaction was allowed to warm to rt and stirred at that temperature overnight. The reaction was poured on ice containing 1N HCl solution (10 mL). The mixture was extracted with ethyl acetate and combined organics washed with brine, and dried over sodium sulfate, filtered and concentrated. The crude was taken up in dichloromethane and purified using normal phase flash chromatography (24 g silica column, Hexane, 0% EtOAc to 80% over 20 Min). This gave the product (11R)-12-[(6-chloro-3-fluoro-2-pyridyl)methyl]-6-(2,6-dimethylphenyl)-11-(2,2-dimethylpropyl)-2,2-dioxo-9-oxa-2⁶-thia-3,5,12,19-tetrazatricyclo[12.3.1.14,8]nonadeca-1(18),4(19),5,7,14,16-hexaen-13-one (303.4 mg, 21%). ESI-MS m/z calc. 637.19257, found 638.4 (M+1)⁺; Retention time: 0.75 minutes; LC method D.

### Step 3: (11R)-6-(2,6-Dimethylphenyl)-11-(2,2-dimethylpropyl)-12-[[3-fluoro-6-[methyl(spiro[2.3]hexan-5-yl)amino]-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1300)

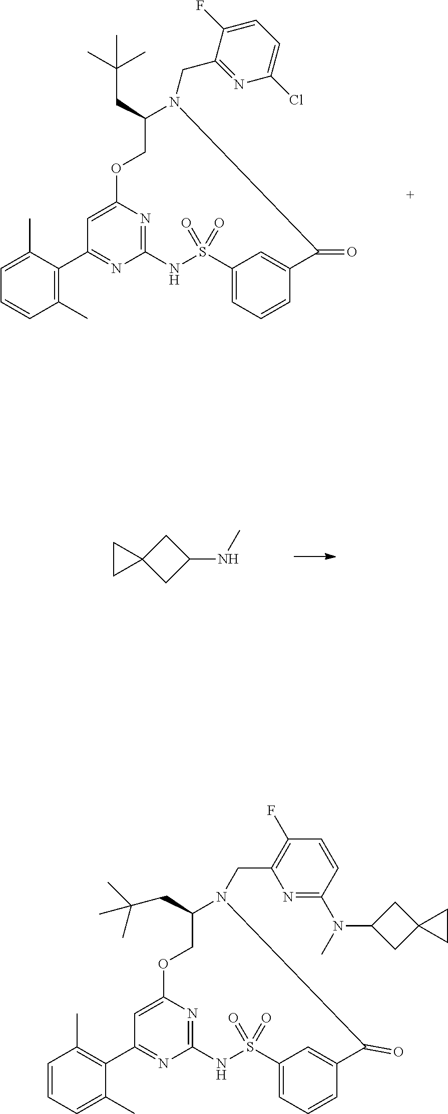

To a solution of (11R)-12-[(6-chloro-3-fluoro-2-pyridyl)methyl]-6-(2,6-dimethylphenyl)-11-(2,2-dimethylpropyl)-2,2-dioxo-9-oxa-2?⁶-thia-3,5,12,19-tetrazatricyclo[12.3.1.14,8]nonadeca-1(18),4(19),5,7,14,16-hexaen-13-one (20 mg, 0.02100 mmol) in NMP (1.4 mL) was added cesium fluoride (31.9 mg, 0.2100 mmol) followed by dimethyl sulfoxide (110 mg, 1.408 mmol). This solution was then heated to 150° C. for 1 h and then was allowed to cool and was followed by the addition of N-methylspiro[2.3]hexan-5-amine (hydrochloride salt) (46.5 mg, 0.3149 mmol) and the solution was then taken to 150° C. and allowed to stir 48 hours. The reaction was extracted with water and EtOAc and the combined organics washed with brine, dried over sodium sulfate, filtered, concentrated and purified by reverse phase HPLC (1-99% MeCN in water with 0.05% TFA) to give (11R)-6-(2,6-dimethylphenyl)-11-(2,2-dimethylpropyl)-12-[[3-fluoro-6-[methyl(spiro[2.3]hexan-5-yl)amino]-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Trifluoroacetate salt) (1.5 mg, 8%). ESI-MS m/z calc. 712.3207, found 713.6 (M+1)⁺; Retention time: 2.14 minutes; LC method A. ¹H NMR (400 MHz, DMSO) δ 7.95 (d, J 6.7 Hz, 1H), 7.68 (s, 2H), 7.43 (t, J 9.3 Hz, 1H), 7.27 (t, J 7.6 Hz, 1H), 7.13 (d, J 7.7 Hz, 2H), 6.54 (dd, J 9.2, 2.6 Hz, 1H), 6.46 (s, 1H), 5.56-5.47 (m, 1H), 5.05 (p, J 8.2 Hz, 1H), 4.34 (dd, J 13.9, 7.4 Hz, 2H), 4.10 (s, 1H), 3.04 (s, 3H), 2.47-2.32 (m, 2H), 2.29-2.09 (m, 2H), 2.01 (s, 7H), 1.77 (dd, J 15.2, 8.8 Hz, 1H), 1.43 (d, J 14.9 Hz, 1H), 1.24 (s, 3H), 0.57 (s, 9H), 0.37 (s, 2H), 0.42-0.31 (m, 1H).

### Example 143: Preparation of Compound 1301

### Step 1: 5-Benzyloxy-2-(chloromethyl)pyrimidine

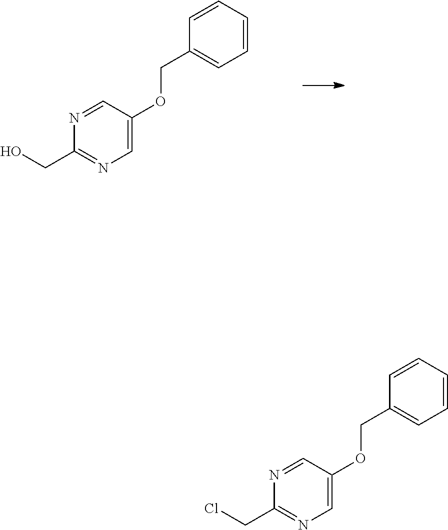 [SMILES: OCc1ncc(OCc2ccccc2)cn1 ; ClCc1ncc(OCc2ccccc2)cn1]

(5-Benzyloxypyrimidin-2-yl)methanol (2.5 g, 11.561 mmol) was dissolved in chloroform (100 mL). Nitrogen was bubbled for ten minutes after which time thionyl chloride (4.8930 g, 3 mL, 41.128 mmol) was added. The reaction was stirred at room temperature for 3 h. The reaction was concentrated under reduced pressure and the white residue was co-evaporated with chloroform (5×10 mL) then dried under high vacuum to afford 5-benzyloxy-2-(chloromethyl)pyrimidine (hydrochloride salt) (3.07 g, 98%) as an off-white solid. ¹H NMR (400 MHz, DMSO-d₆) δ 8.60 (s, 2H), 7.61-7.21 (m, 5H), 6.93-6.51 (m, 1H). 5.30 (s, 2H), 4.76 (s, 2H), ESI-MS m/z calc. 234.05598, found 235.2 (M+1)⁺; Retention time: 1.74 minutes; LC method X.

### Step 2: (11R)-12-[(5-Benzyloxypyrimidin-2-yl)methyl]-6-(2,6-dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

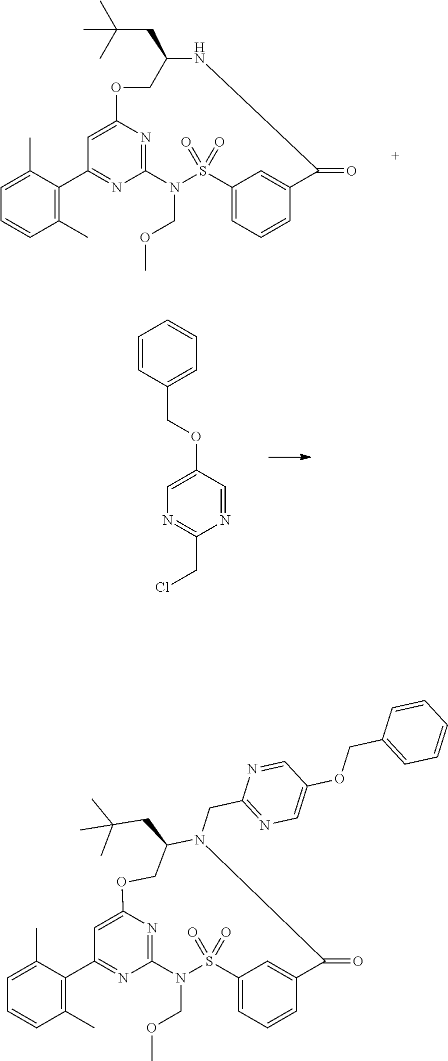 [SMILES: COCN1c2nc(cc(-c3c(C)cccc3C)n2)OC[C@@H](CC(C)(C)C)NC(=O)c2cccc(c2)S1(=O)=O ; ClCc1ncc(OCc2ccccc2)cn1 ; COCN1c2nc(cc(-c3c(C)cccc3C)n2)OC[C@@H](CC(C)(C)C)N(Cc2ncc(OCc3ccccc3)cn2)C(=O)c2cccc(c2)S1(=O)=O]

To a stirred solution of (11R)-6-(2,6-dimethylphenyl)-11-(2,2-dimethylpropyl)-3-(methoxymethyl)-2,2-dioxo-9-oxa-2?⁶-thia-3,5,12,19-tetrazatricyclo[12.3.1.14,8]nonadeca-1(18),4(19),5,7,14,16-hexaen-13-one (1.02 g, 1.8936 mmol) in dry tetrahydrofuran (45 mL) were successively added 5-benzyloxy-2-(chloromethyl)pyrimidine (490 mg, 2.0879 mmol), tetrabutylammonium iodide (150 mg, 0.4061 mmol) and cesium carbonate (2.5 g, 7.6730 mmol) Nitrogen was bubbled and the reaction was stirred overnight at 50° C. Additional cesium carbonate (1.25 g, 3.8365 mmol) was added and the reaction was stirred at 50° C. for 24 h. A saturated aqueous solution of saturated ammonium chloride (200 mL) was added, and the aqueous layer was extracted with ethyl acetate (5×75 mL). The combined organic extracts were washed with brine (250 mL) and water (250 mL), dried over sodium sulfate, filtered, and concentrated under reduced pressure to afford a yellow foam which was purified by reverse phase chromatography on a C₁₈ using a 120 g column and eluting with a gradient of acetonitrile in acidic water containing 0.1% weight of formic acid (5 to 100% in 10 CV then 100% for 3 CV). The desired fractions were concentrated under reduced pressure and residual water was co-evaporated with acetonitrile (3×10 mL) and freeze-dried to afford (11R)-12-[(5-benzyloxypyrimidin-2-yl)methyl]-6-(2,6-dimethylphenyl)-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.04 g, 73%) as a white powder. ¹H NMR (400 MHz, DMSO-d₆) δ 8.85 (br. s, 1H), 8.61 (s, 2H), 8.19-8.12 (m, 1H), 7.85-7.77 (m, 2H), 7.51-7.45 (m, 2H), 7.44-7.38 (m, 2H), 7.38-7.32 (m, 1H), 7.26-7.19 (m, 1H), 7.11 (br. d, J 7.6 Hz, 2H), 6.72 (s, 1H), 5.74 (d, J 10.8 Hz, 1H), 5.57 (d, J 11.0 Hz, 1H), 5.33-5.24 (m, 3H), 4.88 (d, J 16.6 Hz, 1H), 4.70 (d, J 16.6 Hz, 1H), 4.28-4.09 (m, 2H), 2.97 (s, 3H), 2.05-1.87 (m, 6H), 1.78 (dd, J 15.0, 8.2 Hz, 1H), 1.44 (d, J 14.4 Hz, 1H), 0.53 (s, 9H). ESI-MS m/z calc. 736.3043, found 369.0 (M-367)⁺; Retention time: 2.46 minutes; LC method K.

### Step 3: (11R)-6-(2,6-Dimethylphenyl)-11-(2,2-dimethylpropyl)-12-[(5-hydroxypyrimidin-2-yl)methyl]-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

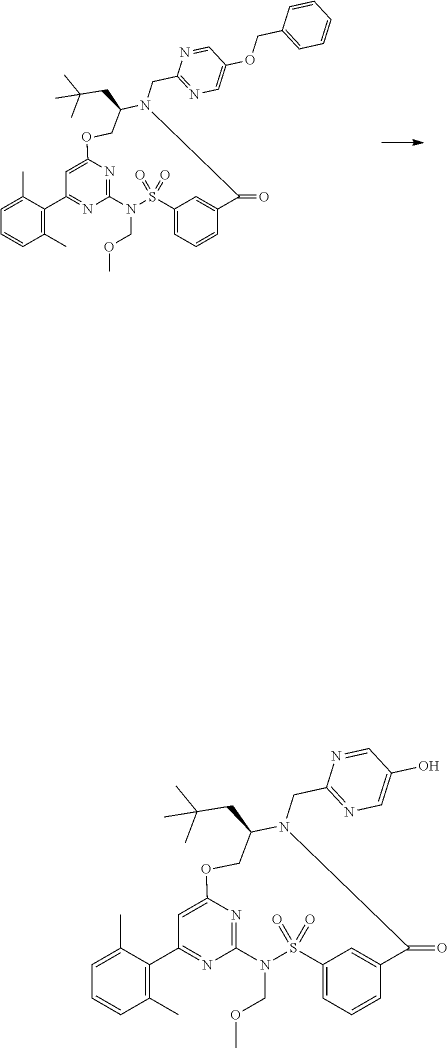 [SMILES: COCN1c2nc(cc(-c3c(C)cccc3C)n2)OC[C@@H](CC(C)(C)C)N(Cc2ncc(OCc3ccccc3)cn2)C(=O)c2cccc(c2)S1(=O)=O ; COCN1c2nc(cc(-c3c(C)cccc3C)n2)OC[C@@H](CC(C)(C)C)N(Cc2ncc(O)cn2)C(=O)c2cccc(c2)S1(=O)=O]

Palladium 5% on carbon (350 mg, 0.1644 mmol) was added into a 250 mL-flask and it was purged with nitrogen for 2 minutes. A solution of (11R)-12-[(5-benzyloxypyrimidin-2-yl)methyl]-6-(2,6-dimethylphenyl)-11-(2,2-dimethylpropyl)-3-(methoxymethyl)-2,2-dioxo-9-oxa-2?⁶-thia-3,5,12,19-tetrazatricyclo[12.3.1.14,8]nonadeca-1(18),4(19),5,7,14,16-hexaen-13-one (1.04 g, 1.4114 mmol) in MeOH (100 mL) was then added into the flask. Hydrogen was bubbled into the suspension for 2 minutes and then the reaction mixture was stirred under 1 atm. of hydrogen for 30 minutes. Nitrogen was then bubbled into the mixture for 10 minutes. The reaction mixture was filtered on Celite, the pad was rinsed with methanol (50 mL) and the filtrate was concentrated in vacuo. The crude was purified on a 80 g C₁₈ GOLD cartridge, eluting with a gradient of MeCN in acidic water (0.1% v/v of formic acid in water) (5% for 5 CV then 50 to 100% for 20 CV). Afforded after evaporation and lyophilization (11R)-6-(2,6-dimethylphenyl)-11-(2,2-dimethylpropyl)-12-[(5-hydroxypyrimidin-2-yl)methyl]-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (731.8 mg, 80%) as a white solid. ESI-MS m/z calc. 646.2574, found 647.0 (M+1)⁺; Retention time: 3.39 minutes; LC method K.

### Step 4: (11R)-6-(2,6-Dimethylphenyl)-12-[[5-[(1S)-1,2-dimethylpropoxy]pyrimidin-2-yl]methyl]-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1301)

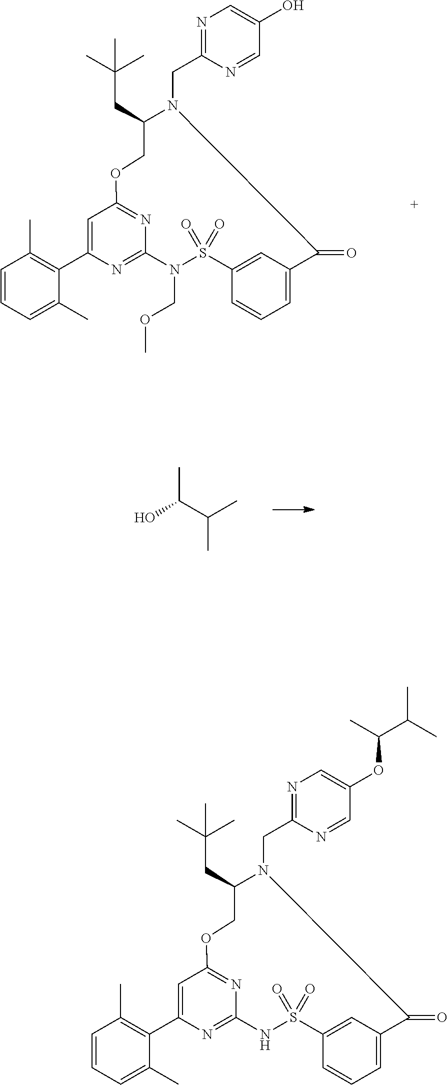

To a solution of (11R)-6-(2,6-dimethylphenyl)-11-(2,2-dimethylpropyl)-12-[(5-hydroxypyrimidin-2-yl)methyl]-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (38 mg, 0.05875 mmol), (2R)-3-methylbutan-2-ol (7 mg, 0.07941 mmol), triphenyl phosphine (20 mg, 0.07625 mmol) in tetrahydrofuran (750 μL) was added DIAD (16 μL, 0.08261 mmol) and the mixture was allowed to stir at room temperature for 90 minutes. The residue was purified by reverse-phase preparative chromatography utilizing a C₁₈ column a 30-99% over 15 min gradient of acetonitrile in water containing 5 mmolar HCl. The desired fractions were pooled and concentrated to afford the desired intermediate (11R)-6-(2,6-dimethylphenyl)-12-[[5-[(]S)-1,2-dimethylpropoxy]pyrimidin-2-yl]methyl]-11-(2,2-dimethylpropyl)-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one, ESI-MS m/z calc. 716.33563, found 717.2 (M+1)⁺; Retention time: 2.02 minutes. To the isolated material was added HCl (4M in dioxane) (approximately 220.3 μL of 4 M, 0.8812 mmol) in DCM (750 μL) and then the mixture was stirred at room temperature for 15 minutes. The mixture was purified by reverse-phase preparative chromatography utilizing a Cis column and a 1-99% over 15 min gradient of acetonitrile in water containing 5 mmolar HCl. The desired fractions were pooled and concentrated to afford the single enantiomer as a white solid, (11R)-6-(2,6-dimethylphenyl)-12-[[5-[(]S)-1,2-dimethylpropoxy]pyrimidin-2-yl]methyl]-11-(2,2-dimethylpropyl)-2,2-dioxo-9-oxa-2?⁶-thia-3,5,12,19-tetrazatricyclo[12.3.1.14,8]nonadeca-1(18),4(19),5,7,14,16-hexaen-13-one (12.3 mg, 31%), ¹H NMR (400 MHz, DMSO-d₆) δ 13.08 (s, 1H), 8.70 (s, 1H), 8.53 (s, 2H), 7.95 (s, 1H), 7.67 (s, 2H), 7.30-7.20 (m, 1H), 7.12 (d, J 7.6 Hz, 2H), 6.42 (s, 1H), 5.43-5.31 (m, 1H), 4.85 (d, J 16.5 Hz, 1H), 4.65 (d, J 16.6 Hz, 1H), 4.47 (p, J 6.1 Hz, 1H), 4.20 (t, J 11.2 Hz, 1H), 4.10-4.01 (m, 1H), 2.25-1.90 (m, 6H), 1.91-1.86 (m, 1H), 1.77 (dd, J 15.1, 8.8 Hz, 1H), 1.40 (d, J 15.0 Hz, 1H), 1.20 (d, J 6.1 Hz, 3H), 0.94 (dd, J=13.7, 6.8 Hz, 6H), 0.56 (s, 9H). ESI-MS m/z calc. 672.3094, found 673.2 (M+1)⁺; Retention time: 2.05 minutes; LC method A.

### Example 144: Preparation of Compound 1302

### Step 1: Methyl 4-ethylpyrimidine-2-carboxylate

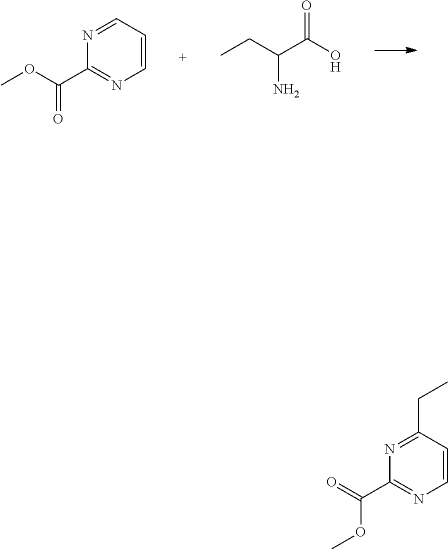

To a mixture of methyl pyrimidine-2-carboxylate (0.5 g, 3.6199 mmol), 2-aminobutanoic acid (749 mg, 7.2634 mmol) and ammonium persulfate (4.1 g, 17.967 mmol) in a mixture of dichloroethane (18 mL) and water (16 mL) was added trifluoroacetic acid (414.40 mg, 0.28 mL, 3.6344 mmol). The mixture was stirred for one minute at room temperature. A silver nitrate aqueous solution (1.8 mL of 2 M, 3.6000 mmol) was added in one portion, and the mixture was stirred at 80° C. for 24 hours. The mixture was quenched with 2 M sodium hydroxide solution (20 mL) and the phase were separated. The aqueous layer was extracted with ethyl acetate (3×30 mL), the organic phase was dried over sodium sulfate, filtered, and concentrated to dryness. The crude was purified by flash chromatography on silica gel (gold 40 g) eluting with 5% to 80% ethyl acetate in heptanes to give methyl 4-ethylpyrimidine-2-carboxylate (290 mg, 43%) as a yellow oil. ESI-MS m/z calc. 166.07423, found 167.2 (M+1)⁺; Retention time: 1.23 minutes; LC method X.

### Step 2: 4-Ethylpyrimidine-2-carbaldehyde

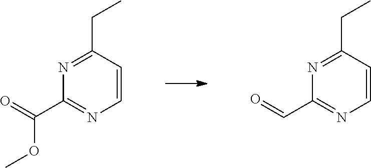

To a solution of methyl 4-ethylpyrimidine-2-carboxylate (290 mg, 1.5462 mmol) in THF (9 mL) was slowly added DIBAL solution in toluene (2 mL of 1 M, 2.0000 mmol) at −60° C. for 40 minutes. The mixture was quenched with a saturated aqueous solution of Rochelle salt (5 mL), the mixture was slowly warmed up to room temperature and stirred overnight. The mixture was extracted with ethyl acetate (4×10 mL). The combined organic layers were washed once with brine (10 mL) and water (10 mL), dried over sodium sulfate, filtered and concentrated to dryness to give crude 4-ethylpyrimidine-2-carbaldehyde (82 mg, 39%) as a pale yellow oil. The crude was directly used for the next step.

### Step 3: 3-[[4-(2,6-Dimethylphenyl)-6-[(2R)-2-[(4-ethylpyrimidin-2-yl)methylamino]-4,4-dimethyl-pentoxy]pyrimidin-2-yl]sulfamoyl]benzoic acid

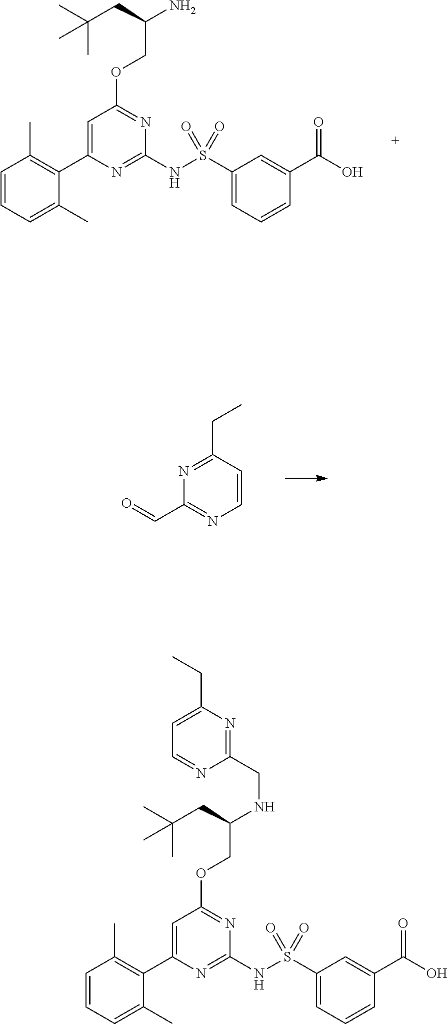

To a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (100 mg, 0.1761 mmol) and 4-ethylpyrimidine-2-carbaldehyde (40 mg, 0.2938 mmol) in anhydrous dichloromethane (2 mL) was added sodium triacetoxyborohydride (116 mg, 0.5473 mmol). The mixture was briefly purged with nitrogen and stirred at room temperature for 1 hour under nitrogen. More 4-ethylpyrimidine-2-carbaldehyde (14 mg, 0.1028 mmol) and sodium triacetoxyborohydride (23 mg, 0.1085 mmol) were added a the mixture was stirred at room temperature for 20 minutes. Then the mixture was quenched with hydrochloric acid solution (4 mL of 1.0 M) and extracted with dichloromethane (2×5 mL). The combined organics were dried over sodium sulfate, filtered, and concentrated under reduced pressure. The crude was purified by reverse phase chromatography (C₁₈, 30 g column) eluting with 5% to 100% acetonitrile in acidic water (containing 0.1% w/w of HCl). The fractions containing the product were concentrated to remove the acetonitrile, diluted hydrochloric acid (10 mL of 1.0 M) and extracted with ethyl acetate (3×10 mL). The organic phase was dried over sodium sulfate, filtered and concentrated to give 3-[[4-(2,6-dimethylphenyl)-6-[(2R)-2-[(4-ethylpyrimidin-2-yl)methylamino]-4,4-dimethyl-pentoxy]pyrimidin-2-yl]sulfamoyl]benzoic acid (hydrochloride salt) (68 mg, 50%) as an off-white solid. ESI-MS m/z calc. 632.2781, found 633.2 (M+1)⁺; Retention time: 1.46 minutes; LC method 1D.

### Step 4: (11R)-6-(2,6-Dimethylphenyl)-11-(2,2-dimethylpropyl)-12-[(4-ethylpyrimidin-2-yl)methyl]-2,2-dioxo-9-oxa-2λ⁶-thia-3,5,12,19-tetrazatricyclo[12.3.1.14,8]nonadeca-1(18),4,6,8(19),14,16-hexaen-13-one (Compound 1302)

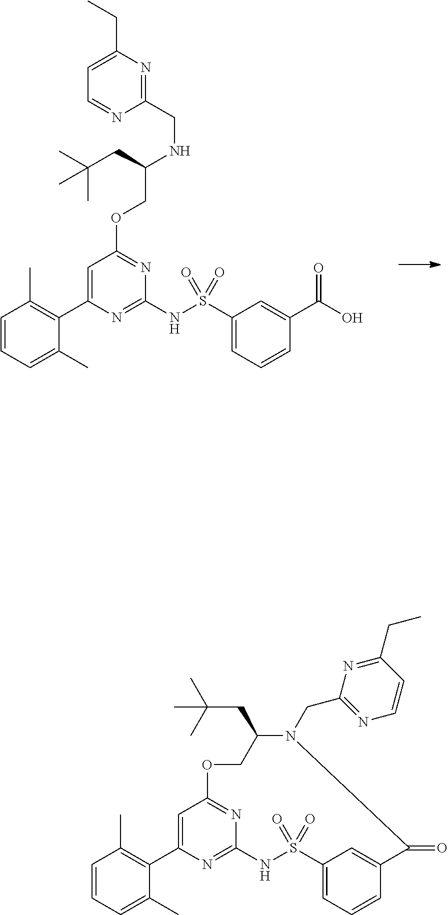 [SMILES: CCc1ccnc(CN[C@@H](COc2cc(-c3c(C)cccc3C)nc(NS(=O)(=O)c3cccc(C(=O)O)c3)n2)CC(C)(C)C)n1 ; CCc1ccnc(CN2C(=O)c3cccc(c3)S(=O)(=O)Nc3nc(cc(-c4c(C)cccc4C)n3)OC[C@H]2CC(C)(C)C)n1]

To a solution of 3-[[4-(2,6-dimethylphenyl)-6-[(2R)-2-[(4-ethylpyrimidin-2-yl)methylamino]-4,4-dimethyl-pentoxy]pyrimidin-2-yl]sulfamoyl]benzoic acid (68 mg, 0.0928 mmol) and N-methylmorpholine (64.400 mg, 70 μL, 0.6367 mmol) in DMF (6.8 mL) was added 2-chloro-4,6-dimethoxy-1,3,5-triazine (38 mg, 0.2164 mmol) at 0° C. After stirring 5 minutes at this temperature, the ice bath was removed, and the mixture was then stirred at room temperature for 18 hours. The mixture was concentrated to dryness at 50° C. and the crude was purified by reverse phase chromatography (gold C₁₈, 30 g) eluting with a gradient of 5% to 100% acetonitrile in acidic water (containing 0.1% v/v of formic acid) to give (11R)-6-(2,6-dimethylphenyl)-11-(2,2-dimethylpropyl)-12-[(4-ethylpyrimidin-2-yl)methyl]-2,2-dioxo-9-oxa-2λ⁶-thia-3,5,12,19-tetrazatricyclo[12.3.1.14,8]nonadeca-1(18),4,6,8(19),14,16-hexaen-13-one (28 mg, 46%) as a white powder after lyophilization. ¹H NMR (400 MHz, DMSO-d₆) δ 13.06 (br. s., 1H), 8.81 (br. s., 1H), 8.66 (d, J 5.1 Hz, 1H), 7.94 (br. s., 1H), 7.67 (br. s., 2H), 7.31-7.21 (m, 2H), 7.18-7.07 (m, 2H), 6.40 (br. s., 1H), 5.51 (d, J 6.8 Hz, 1H), 4.87 (d, J 17.1 Hz, 1H), 4.70 (d, J 17.1 Hz, 1H), 4.18 (t, J 11.1 Hz, 1H), 4.06-3.94 (m, 1H), 2.78 (q, J 7.5 Hz, 2H), 2.26-1.85 (m, 6H), 1.77 (dd, J 15.4, 9.0 Hz, 1H), 1.42 (d, J 14.4 Hz, 1H), 1.26 (t, J 7.6 Hz, 3H), 0.59 (s, 9H). ESI-MS m/z calc. 614.2675, found 615.3 (M+1)⁺; Retention time: 4.23 minutes; LC method Y.

### Example 145: Preparation of Compound 1303

### Step 1: 3-Iodo-1H-pyrrolo[2,3-b]pyridine-6-carbaldehyde

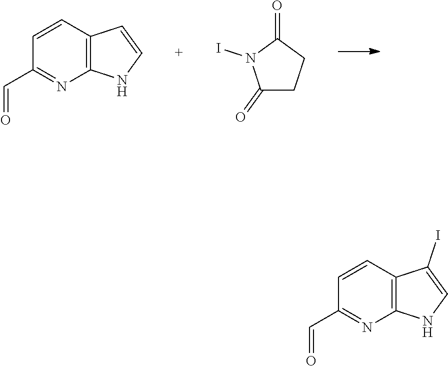 [SMILES: O=Cc1ccc2cc[nH]c2n1 ; O=C1CCC(=O)N1I ; O=Cc1ccc2c(I)c[nH]c2n1]

A microwave vial was charged with 1H-pyrrolo[2,3-b]pyridine-6-carbaldehyde (500 mg, 3.4212 mmol) NIS (850 mg, 3.7780 mmol) dissolved in DMF (5 mL) and it was flushed with nitrogen, capped and then left stirring at room temperature overnight. Water (10 mL) was added to and the the resulting precipitate was filtered and washed with water (10 mL) and then with methanol (3×3 mL) and dried under high vacuum to provide 3-iodo-1H-pyrrolo[2,3-b]pyridine-6-carbaldehyde (873 mg, 94%) as a yellow powder. ESI-MS m/z calc. 271.94464, found 273.0 (M+1)⁺; Retention time: 1.63 minutes; LC method X.

### Step 2: 3-Iodo-1-methyl-pyrrolo[2,3-b]pyridine-6-carbaldehyde

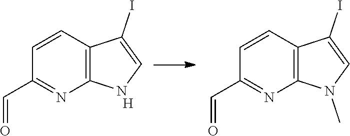

Methyl iodide (684.00 mg, 0.3 mL, 4.8190 mmol) was added to a suspension of 3-iodo-1H-pyrrolo[2,3-b]pyridine-6-carbaldehyde (425 mg, 1.5607 mmol) and potassium carbonate (435 mg, 3.1475 mmol) in DMF (10 mL) and left stirring overnight. Water (50 mL) was added to the reaction mixture and the precipitate that formed was filtered and washed with distilled water then with a small amount of methanol then dried under high vacuum to provide pure 3-iodo-1-methyl-pyrrolo[2,3-b]pyridine-6-carbaldehyde (408 mg, 91%) as a pale yellow powder, ¹H NMR (400 MHz, CDCl₃) δ 10.14 (s, 1H), 7.88 (d, J 8.1 Hz, 1H), 7.84 (d, J 8.3 Hz, 1H), 7.55 (s, 1H), 4.01 (s, 3H). ESI-MS m/z calc. 285.9603, found 287.0 (M+1)⁺; Retention time: 1.79 minutes; LC method X.

### Step 3: 3-[[4-(2,6-Dimethylphenyl)-6-[(2R)-2-[(3-iodo-1-methyl-pyrrolo[2,3-b]pyridin-6-yl)methylamino]-4,4-dimethyl-pentoxy]pyrimidin-2-yl]sulfamoyl]benzoic acid

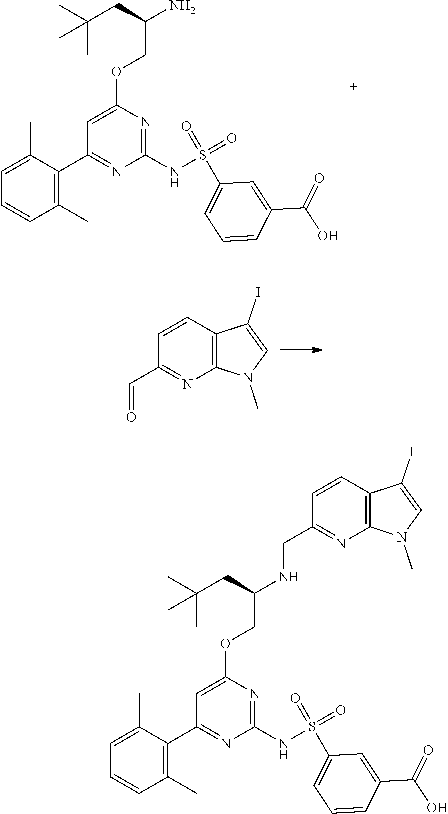 [SMILES: Cc1cccc(C)c1-c1cc(OC[C@H](N)CC(C)(C)C)nc(NS(=O)(=O)c2cccc(C(=O)O)c2)n1 ; "+" ; Cn1cc(I)c2ccc(C=O)nc21 ; Cc1cccc(C)c1-c1cc(OC[C@@H](CC(C)(C)C)NCc2ccc3c(I)cn(C)c3n2)nc(NS(=O)(=O)c2cccc(C(=O)O)c2)n1]

3-[[4-[(2R)-2-Amino-4,4-dimethyl-pentoxy]-6-(2,6-dimethylphenyl)pyrimidin-2-yl]sulfamoyl]benzoic acid (hydrochloride salt) (520 mg, 0.9470 mmol) and 3-iodo-1-methyl-pyrrolo[2,3-b]pyridine-6-carbaldehyde (286 mg, 0.9998 mmol) were stirred together in dichloromethane (25 mL) at 19-20° C. for 2 h then sodium triacetoxyborohydride (921 mg, 4.3455 mmol) was added in one portion and the mixture was stirred at this temperature for 1 h, then quenched with HCl 1N (25 mL) at 0° C. and stirred at this temperature for 20 min. The aqueous phase was separated and washed with DCM (50 mL). The combined organic phases were dried with sodium sulfate, filtered, and concentrated under reduced pressure. The resulting residue was purified by reverse phase chromatography on a 50 g C₁₈ column using a 5 to 100% gradient of acetonitrile in acidic water (10% v/v of HCl 3N in water) to provide 3-[[4-(2,6-dimethylphenyl)-6-[(2R)-2-[(3-iodo-1-methyl-pyrrolo[2,3-b]pyridin-6-yl)methylamino]-4,4-dimethyl-pentoxy]pyrimidin-2-yl]sulfamoyl]benzoic acid (hydrochloride salt) (581 mg, 73%) as a pale yellow solid. ESI-MS m/z calc. 782.17474, found 782.8 (M+1)⁺; Retention time: 1.57 minutes; LC method X.

### Step 4: (11R)-6-(2,6-Dimethylphenyl)-11-(2,2-dimethylpropyl)-12-[(3-iodo-1-methyl-pyrrolo[2,3-b]pyridin-6-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

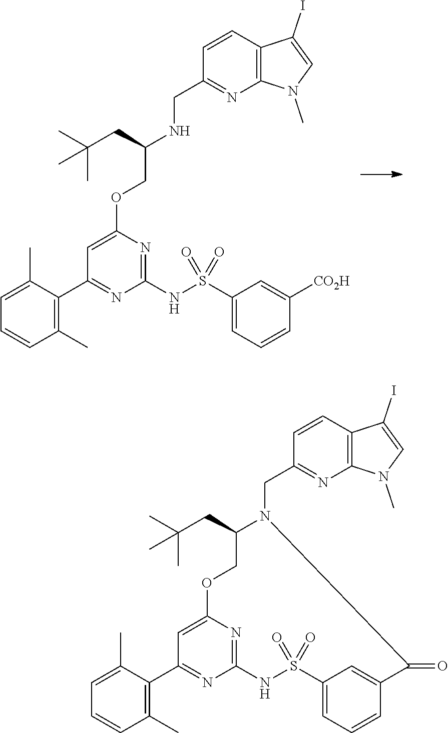 [SMILES: Cc1cccc(C)c1-c1cc(OC[C@@H](CC(C)(C)C)NCc2ccc3c(I)cn(C)c3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccc3c(I)cn(C)c3n1)[C@H](CC(C)(C)C)CO2]

DIPEA (259.70 mg, 0.35 mL, 2.0094 mmol) was added dropwise to a solution of 3-[[4-(2,6-dimethylphenyl)-6-[(2R)-2-[(3-iodo-1-methyl-pyrrolo[2,3-b]pyridin-6-yl)methylamino]-4,4-dimethyl-pentoxy]pyrimidin-2-yl]sulfamoyl]benzoic acid (220 mg, 0.2811 mmol) in DMF (8 mL) at 0° C., 2-Chloro-1-methylpyridinium iodide (145 mg, 0.5676 mmol) was added and the reaction mixture was left stirring at room temperature overnight. The mixture was concentrated under reduced pressure (50° C.). The resulting residue was dissolved in a small amount of DMSO, filtered and purified by reverse phase chromatography on a 50 g C₁₈ using a 5 to 100% gradient of acetonitrile in basic water (ammonium bicarbonate/ammonium hydroxide buffer pH=10) to provide (11R)-6-(2,6-dimethylphenyl)-11-(2,2-dimethylpropyl)-12-[(3-iodo-1-methyl-pyrrolo[2,3-b]pyridin-6-yl)methyl]-2,2-dioxo-9-oxa-2?⁶-thia-3,5,12,19-tetrazatricyclo[12.3.1.14,8]nonadeca-1(18),4(19),5,7,14,16-hexaen-13-one (132 mg, 61%) as a beige powder after lyophilization. ¹H NMR (400 MHz, DMSO-d₆) δ 13.07 (br. s., 1H), 8.88 (br. s., 1H), 7.94 (br. s., 1H), 7.74-7.62 (m, 4H), 7.26 (d, J 8.1 Hz, 2H), 7.17-7.05 (m, 2H), 6.39 (br. s., 1H), 5.60 (dd, J 10.1, 3.3 Hz, 1H), 5.06 (d, J 16.4 Hz, 1H), 4.69 (d, J 16.1 Hz, 1H), 4.18 (t, J 10.9 Hz, 1H), 4.08-3.99 (m, 1H), 3.90 (s, 3H), 2.24-1.86 (m, 6H), 1.81 (dd, J 15.0, 8.9 Hz, 1H), 1.46 (d, J=14.9 Hz, 1H), 0.60 (s, 9H). ESI-MS m/z calc. 764.1642, found 765.8 (M+1)⁺; Retention time: 2.1 minutes; LC method X.

### Step 5: (11R)-12-[(3-Cyclopropyl-1-methyl-pyrrolo[2,3-b]pyridin-6-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1303)

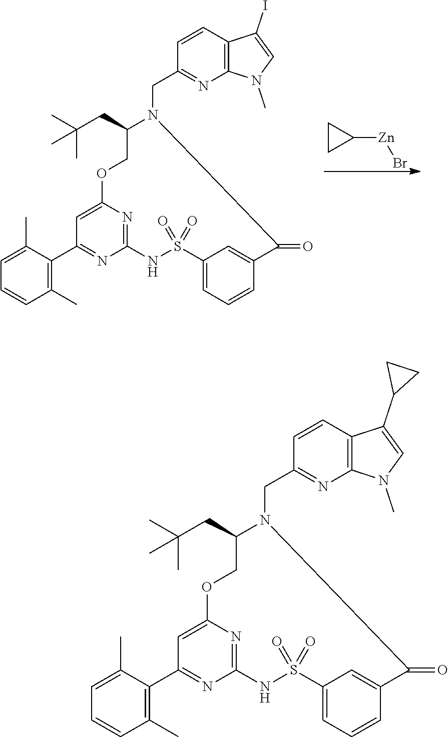

A microwave vial was charged with (11R)-6-(2,6-dimethylphenyl)-11-(2,2-dimethylpropyl)-12-[(3-iodo-1-methyl-pyrrolo[2,3-b]pyridin-6-yl)methyl]-2,2-dioxo-9-oxa-2?⁶-thia-3,5,12,19-tetrazatricyclo[12.3.1.14,8]nonadeca-1(18),4(19),5,7,14,16-hexaen-13-one (11 mg, 0.0144 mmol), 1,1′-Bis(diphenylphosphino)ferrocene palladium(II) chloride, complex with dichloromethane (3 mg, 0.0037 mmol) and bromo(cyclopropyl)zinc (0.5 mL of 0.5 M, 0.2500 mmol). Nitrogen gas was injected into the vial above the reaction mixture. The vial was capped and heated in an oil bath at 60° C. overnight. The reaction mixture was concentrated with a flow of nitrogen, then quenched with a 1:1 mixture of acetonitrile, water (0.2 mL). More acetonitrile was added (1 mL) and the solution was filtered on a syringe micro filter then concentrated under reduced pressure and purified by reverse phase chromatography on a 20 g C₁₈ column using a 5 to 100% gradient of acetonitrile in basic water (ammonium bicarbonate/ammonium hydroxide buffer pH=10) to provide (11R)-12-[(3-cyclopropyl-1-methyl-pyrrolo[2,3-b]pyridin-6-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8] nonadeca-1(18),4(19),5,7,14,16-hexaen-13-one (6 mg, 61%) as a white fluffy solid after lyophilization. ¹H NMR (400 MHz, DMSO-d₆) δ 13.05 (br. s., 1H), 8.86 (br. s., 1H), 8.02-7.89 (m, 2H), 7.66 (br. s., 2H), 7.25 (m, 1H), 7.16 (s, 1H), 7.15-7.06 (m, 3H), 6.47-6.30 (m, 1H), 5.59 (d, J 8.8 Hz, 1H), 5.03 (d, J 16.1 Hz, 1H), 4.62 (d, J 16.1 Hz, 1H), 4.23-4.10 (m, 1H), 4.08-3.99 (m, 1H), 3.80 (s, 3H), 2.23-1.88 (m, 7H), 1.82 (dd, J 15.2, 8.3 Hz, 1H), 1.46 (d, J=15.2 Hz, 1H), 0.89-0.81 (m, 2H), 0.60 (m, 11H). ESI-MS m/z calc. 678.2988, found 679.3 (M+1)⁺; Retention time: 4.97 minutes; LC method Y.

### Example 146: Preparation of Compound 1304

### Step 1: 6-Bromo-1-methyl-pyrrolo[2,3-b]pyridine

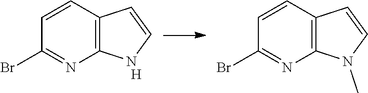

Iodomethane (10 g, 70.453 mmol) was added to 6-bromo-1H-pyrrolo[2,3-b]pyridine (5 g, 25.351 mmol) and potassium carbonate (7 g, 3.0702 mL, 50.649 mmol) in suspension in DMF (100 mL) and left stirring at room temperature overnight. The reaction mixture was concentrated under reduced pressure (at 50° C.) and the resulting residue was partitioned between diethyl ether (300 mL) and water (100 mL). The aqueous phase was separated and extracted with more diethyl ether (200 mL). The combined organic phases was washed with brine (3×100 mL) to remove residual DMF then it was dried with sodium sulfate, filtered and concentrated under reduced pressure to provide pure 6-bromo-1-methyl-pyrrolo[2,3-b]pyridine (5.29 g, 99%) as a yellow oil. ¹H NMR (400 MHz, CDCl₃) δ 7.75 (d, J 8.1 Hz, 1H), 7.20 (d, J 8.3 Hz, 1H), 7.14 (d, J 3.7 Hz, 1H), 6.44 (d, J 3.7 Hz, 1H), 3.87 (s, 3H). ESI-MS m/z calc. 209.97926, found 211.0 (M+1)⁺; Retention time: 1.77 minutes; LC method X.

### Step 2: 1-Methylpyrrolo[2,3-b]pyridine-6-carbaldehyde

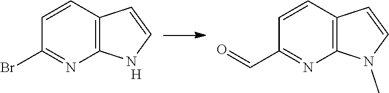

n-BuLi (2.5 M in hexane) (4.8 mL of 2.5 M, 12.000 mmol) was slowly added to a solution of 6-bromo-1-methyl-pyrrolo[2,3-b]pyridine (1.74 g, 8.2441 mmol) in THF (40 mL) maintained at −78° C. Then stirred at that temperature for 45 min. DMF (1.2272 g, 1.3 mL, 16.789 mmol) was added to the reaction mixture which was then left to warm-up to room temperature over ˜ 45 min and quenched with aqueous saturated ammonium chloride (40 mL). Diethyl ether (200 mL) was added, and the aqueous phase was separated and extracted with diethyl ether (100 mL). The combined organic phases were washed with distilled water, (40 mL) then brine (3×50 mL). The organic phase was dried with sodium sulfate, filtered and concentrated under reduced pressure to provide 1-methylpyrrolo[2,3-b]pyridine-6-carbaldehyde (1.2 g, 81%) as a yellow oil which partially crystallized on standing. ¹H NMR (400 MHz, CDCl₃) δ 10.12 (s, 1H), 8.01 (d, J 8.1 Hz, 1H), 7.81 (d, J 8.1 Hz, 1H), 7.44 (d, J 3.4 Hz, 1H), 6.56 (d, J=3.4 Hz, 1H), 3.98 (s, 3H). ESI-MS m/z calc. 160.06366, found 161.2 (M+1)⁺; Retention time: 1.55 minutes; LC method X.

### Step 3: (E)-N-tert-butyl-1-(1-methylpyrrolo[2,3-b]pyridin-6-yl)methanimine

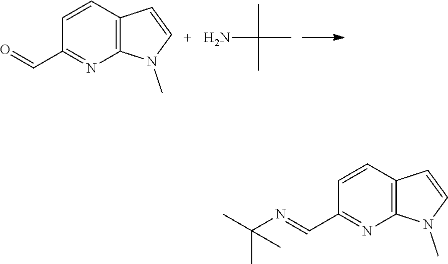

A microwave vial was charged with 1-methylpyrrolo[2,3-b]pyridine-6-carbaldehyde (510 mg, 3.1841 mmol) and 2-methylpropan-2-amine (245 mg, 3.3499 mmol), flushed with nitrogen gas then capped. The reaction was heated at 100° C. overnight in an oil bath. The reaction mixture was diluted with diethyl ether, dried with sodium sulfate, filtered and concentrated under reduced pressure to provide as a dark material (E)-N-tert-butyl-1-(1-methylpyrrolo[2,3-b]pyridin-6-yl)methanimine (430 mg, 52%) as a dark solid. ¹H NMR (400 MHz, CDCl₃) δ 8.46 (s, 1H), 7.94-7.88 (m, 2H), 7.24 (d, J 3.4 Hz, 1H), 6.47 (d, J 3.4 Hz, 1H), 3.93 (s, 3H), 1.35 (s, 9H). ESI-MS m/z calc. 215.1422, found 161.0 (M-55)⁺; Retention time: 1.55 minutes; LC method X.

### Step 4: 2-Iodo-1-methyl-pyrrolo[2,3-b]pyridine-6-carbaldehyde

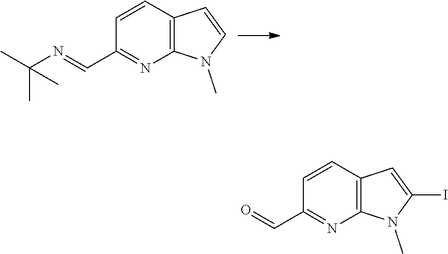 [SMILES: Cn1ccc2ccc(/C=N/C(C)(C)C)nc21 ; Cn1c(I)cc2ccc(C=O)nc21]

LDA (2M in THF, heptane, ethylbenzene) (350 μL of 2 M, 0.7000 mmol) was slowly added to a solution of (E)-N-tert-butyl-1-(1-methylpyrrolo[2,3-b]pyridin-6-yl)methanimine (100 mg, 0.4645 mmol) in THF (5 mL) maintained at −78° C. for 1 h. Iodine (120 mg, 0.0243 mL, 0.4728 mmol) was added dissolved THF (5 mL) and stirred for 30 min. The Reaction mixture was quenched with aqueous sodium sulfite (1 mL), saturated aqueous ammonium chloride (5 mL). The organic phase was separated, and the aqueous phase was extracted with ethyl acetate (2×10 mL). The combined organics was dried with sodium sulfate and concentrated under reduced pressure. The resulting residue was purified by was purified by reverse phase chromatography on 50 g C₁₈ column using a 5 to 100% gradient of ACN in acidic water (0.1% formic acid content) to provide 2-iodo-1-methyl-pyrrolo[2,3-b]pyridine-6-carbaldehyde (28.7 mg, 22%) as a pale yellow powder. ¹H NMR (400 MHz, CDCl₃) δ 10.11 (s, 1H), 7.93 (d, J 8.1 Hz, 1H), 7.77 (d, J 8.1 Hz, 1H), 6.91 (s, 1H), 3.97 (s, 3H). ESI-MS m/z calc. 285.9603, found 287.0 (M+1)⁺; Retention time: 1.8 minutes; LC method X.

### Step 5: 3-[[4-(2,6-Dimethylphenyl)-6-[(2R)-2-[(2-iodo-1-methyl-pyrrolo[2,3-b]pyridin-6-yl)methylamino]-4,4-dimethyl-pentoxy]pyrimidin-2-yl]sulfamoyl]benzoic acid

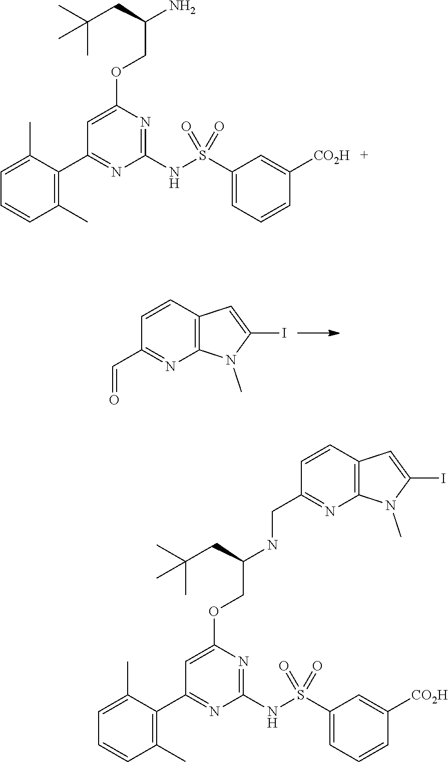

3-[[4-[(2R)-2-Amino-4,4-dimethyl-pentoxy]-6-(2,6-dimethylphenyl)pyrimidin-2-yl]sulfamoyl]benzoic acid (hydrochloride salt) (160 mg, 0.2914 mmol) and 2-iodo-1-methyl-pyrrolo[2,3-b]pyridine-6-carbaldehyde (95 mg, 0.3321 mmol) were stirred together in dichloromethane (4 mL) at 19-20° C. for 2 h then sodium triacetoxyborohydride (310 mg, 1.4627 mmol) was added in one portion and stirred at this temperature for 1 h then the mixture was quenched with HCl 1N (25 mL) at 0° C. and stirred at this temperature for 20 min. The aqueous phase was separated and washed with DCM (50 mL). The combined organic phases were dried over sodium sulfate, filtered and concentrated under reduced pressure. The resulting residue was purified by reverse phase chromatography on a 50 g C₁₈ column using a 5 to 100% gradient of acetonitrile in acidic water (10% v/v of HCl 3N in water) to provide 3-[[4-(2,6-dimethylphenyl)-6-[(2R)-2-[(2-iodo-1-methyl-pyrrolo[2,3-b]pyridin-6-yl)methylamino]-4,4-dimethyl-pentoxy]pyrimidin-2-yl]sulfamoyl]benzoic acid (hydrochloride salt) (130 mg, 51%) as a pale yellow solid. ESI-MS m/z calc. 782.17474, found 783.2 (M+1)⁺; Retention time: 1.57 minutes; LC method X.

### Step 6: (11R)-6-(2,6-Dimethylphenyl)-11-(2,2-dimethylpropyl)-12-[(2-iodo-1-methyl-pyrrolo[2,3-b]pyridin-6-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one

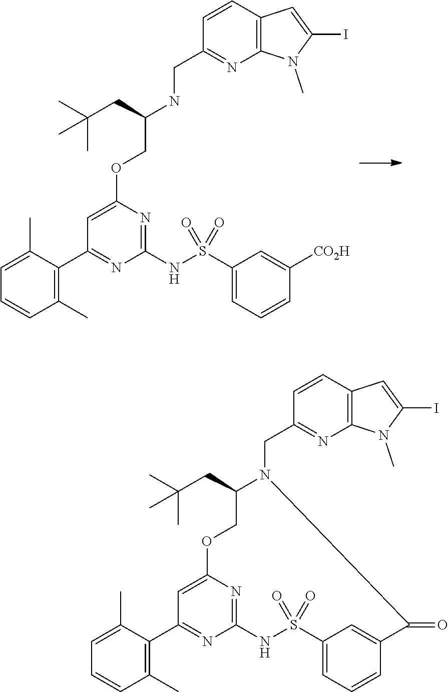 [SMILES: Cc1cccc(C)c1-c1cc(OC[C@@H](CC(C)(C)C)NCc2ccc3cc(I)n(C)c3n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ccc3cc(I)n(C)c3n1)[C@H](CC(C)(C)C)CO2]

DIPEA (150 mg, 0.2022 mL, 1.1606 mmol) was added to a solution of 3-[[4-(2,6-dimethylphenyl)-6-[(2R)-2-[(2-iodo-1-methyl-pyrrolo[2,3-b]pyridin-6-yl)methylamino]-4,4-dimethyl-pentoxy]pyrimidin-2-yl]sulfamoyl]benzoic acid (130 mg, 0.1661 mmol) in DMF (4 mL) at 0° C. 2-Chloro-1-methylpyridinium iodide (85 mg, 0.3327 mmol) was added and the reaction was left stirring at this temperature for 30 min and then left to warm up to room temperature and stirred overnight. The reaction mixture was concentrated and purified by reverse phase chromatography on 50 g C₁₈ column using a 5 to 100% gradient of acetonitrile in basic water (ammonium bicarbonate/ammonium hydroxide buffer pH=10) to provide (11R)-6-(2,6-dimethylphenyl)-11-(2,2-dimethylpropyl)-12-[(2-iodo-1-methyl-pyrrolo[2,3-b]pyridin-6-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (94 mg, 72%) as a white fluffy powder after lyophilization. ¹H NMR (400 MHz, DMSO-d₆) δ 13.08 (br. s., 1H), 8.87 (br. s., 1H), 7.94 (br. s., 1H), 7.88 (d, J 7.8 Hz, 1H), 7.67 (br. s., 2H), 7.31-7.21 (m, 1H), 7.18-7.08 (m, 3H), 6.85 (s, 1H), 6.39 (br. s., 1H), 5.64-5.54 (m, 1H), 5.03 (d, J 16.1 Hz, 1H), 4.64 (d, J 16.1 Hz, 1H), 4.18 (t, J 10.6 Hz, 1H), 4.08-3.99 (m, 1H), 3.85 (s, 3H), 2.23-1.86 (m, 6H), 1.81 (dd, J 14.9, 8.8 Hz, 1H), 1.46 (d, J 14.7 Hz, 1H), 0.60 (s, 9H). ESI-MS m/z calc. 764.1642, found 765.2 (M+1)⁺; Retention time: 4.98 minutes; LC method Y.

### Step 7: (11R)-12-[(2-Cyclopropyl-1-methyl-pyrrolo[2,3-b]pyridin-6-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1304)

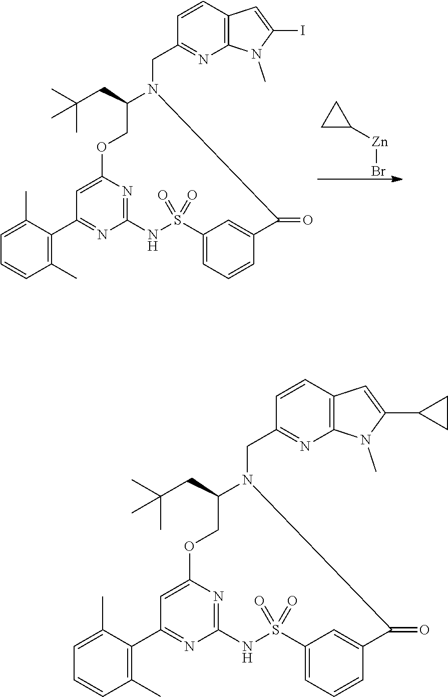

In a microwave tube, under nitrogen gas, bromo(cyclopropyl)zinc (0.5 M in THF) (1.5 mL of 0.5 M, 0.7500 mmol) was added to (11R)-6-(2,6-dimethylphenyl)-11-(2,2-dimethylpropyl)-12-[(2-iodo-1-methyl-pyrrolo[2,3-b]pyridin-6-yl)methyl]-2,2-dioxo-9-oxa-2?⁶-thia-3,5,12,19-tetrazatricyclo[12.3.1.14,8]nonadeca-1(18),4(19),5,7,14,16-hexaen-13-one (30 mg, 0.0383 mmol) and 1,1′-Bis(diphenylphosphino)ferrocene palladium(II) chloride, complex with dichloromethane (10 mg, 0.0122 mmol). The tube was capped, and the reaction mixture was heated at 60° C. in an oil bath with stirring overnight. Water (50 μL) was added to the reaction mixture was then it was filtered with a syringe micro filter and the solvents were evaporated under reduced pressure. The resulting residue was purified by reverse phase chromatography on a 20 g C₁₈ column using a 5 to 100% gradient of acetonitrile in basic water (ammonium bicarbonate/ammonium hydroxide buffer pH=10) to provide (11R)-12-[(2-cyclopropyl-1-methyl-pyrrolo[2,3-b]pyridin-6-yl)methyl]-6-(2,6-dimethylphenyl)-11-(2,2-dimethylpropyl)-2,2-dioxo-9-oxa-2?⁶-thia-3,5,12,19-tetrazatricyclo[12.3.1.14,8]nonadeca-1(18),4(19),5,7,14,16-hexaen-13-one (7 mg, 27%) as a white fluffy solid after lyophilization. ¹H NMR (400 MHz, DMSO-d₆) δ 13.09 (br. s., 1H), 8.89 (br. s., 1H), 7.94 (br. s., 1H), 7.78 (d, J 7.8 Hz, 1H), 7.67 (br. s., 2H), 7.31-7.21 (m, 1H), 7.17-7.04 (m, 3H), 6.39 (br. s., 1H), 6.08 (s, 1H), 5.60 (d, J 7.1 Hz, 1H), 5.01 (d, J 16.1 Hz, 1H), 4.60 (d, J 15.9 Hz, 1H), 4.24-4.12 (m, 1H), 4.10-4.00 (m, 1H), 3.91 (s, 3H), 2.22-1.89 (m, 7H), 1.82 (dd, J 15.2, 8.6 Hz, 1H), 1.46 (d, J 14.9 Hz, 1H), 1.05-0.94 (m, 2H), 0.78-0.65 (m, 2H), 0.60 (s, 9H). ESI-MS m/z calc. 678.2988, found 679.3 (M+1)⁺; Retention time: 4.96 minutes and a second fraction of (11R)-12-[(2-cyclopropyl-1-methyl-pyrrolo[2,3-b]pyridin-6-yl)methyl]-6-(2,6-dimethylphenyl)-11-(2,2-dimethylpropyl)-2,2-dioxo-9-oxa-2?⁶-thia-3,5,12,19-tetrazatricyclo[12.3.1.14,8]nonadeca-1(18),4(19),5,7,14,16-hexaen-13-one (10 mg, 38%) as a white fluffy solid after lyophilization ¹H NMR (400 MHz, DMSO-d₆) δ 13.06 (br. s., 1H), 8.89 (br. s., 1H), 7.94 (br. s., 1H), 7.78 (d, J 7.8 Hz, 1H), 7.67 (br. s., 2H), 7.35-7.20 (m, 1H), 7.18-7.04 (m, 3H), 6.40 (br. s., 1H), 6.08 (s, 1H), 5.61 (d, J 8.1 Hz, 1H), 5.01 (d, J 15.7 Hz, 1H), 4.61 (d, J 15.7 Hz, 1H), 4.29-4.12 (m, 1H), 4.09-4.00 (m, 1H), 3.91 (s, 3H), 2.22-1.89 (m, 7H), 1.82 (dd, J 14.8, 8.9 Hz, 1H), 1.46 (d, J=14.9 Hz, 1H), 1.05-0.92 (m, 2H), 0.76-0.66 (m, 2H), 0.60 (s, 9H). ESI-MS m/z calc. 678.2988, found 679.3 (M+1)⁺; Retention time: 4.96 minutes; LC method Y.

### Example 147: Preparation of Compound 1305

### Step 1: (11R)-12-[[5-(Cyclopentoxy)pyrimidin-2-yl]methyl]-6-(2,6-dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

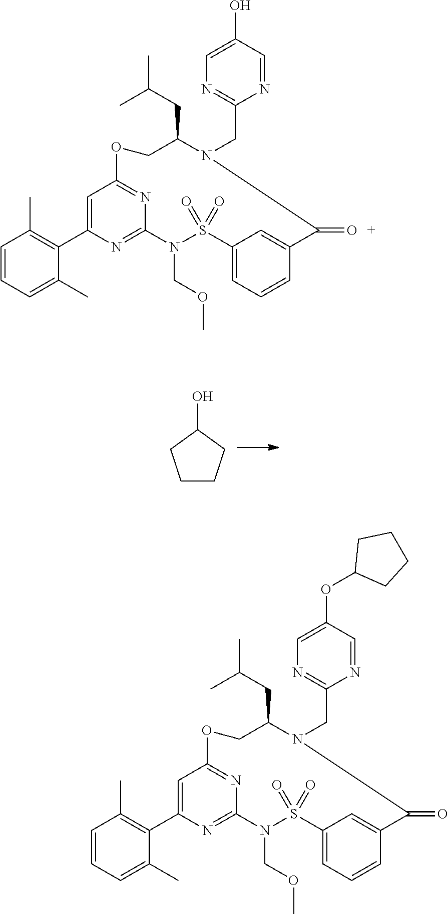 [SMILES: COCN1c2nc(cc(-c3c(C)cccc3C)n2)OC[C@@H](CC(C)C)N(Cc2ncc(O)cn2)C(=O)c2cccc(c2)S1(=O)=O ; OC1CCCC1 ; COCN1c2nc(cc(-c3c(C)cccc3C)n2)OC[C@@H](CC(C)C)N(Cc2ncc(OC3CCCC3)cn2)C(=O)c2cccc(c2)S1(=O)=O]

To (11R)-6-(2,6-dimethylphenyl)-12-[(5-hydroxypyrimidin-2-yl)methyl]-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (33.6 mg, 0.05310 mmol), cyclopentanol (6.4 μL, 0.07044 mmol), and triphenyl phosphine (19.2 mg, 0.07320 mmol) was added was added THF (411 μL) followed by DIAD (14.3 mg, 0.07072 mmol) and the reaction was stirred for 1 hour. The reaction was then diluted with acetonitrile and purified with reverse phase HPLC (1-99 MeCN in H₂O with 0.05% HCl) to give the product (11R)-12-[[5-(cyclopentoxy)pyrimidin-2-yl]methyl]-6-(2,6-dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (16.9 mg, 40%) ESI-MS m/z calc. 700.3043, found 701.5 (M+1)⁺; Retention time: 2.34 minutes; LC method A.

### Step 2: (11R)-12-[[5-(Cyclopentoxy)-1-oxido-pyrimidin-1-ium-2-yl]methyl]-6-(2,6-dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one

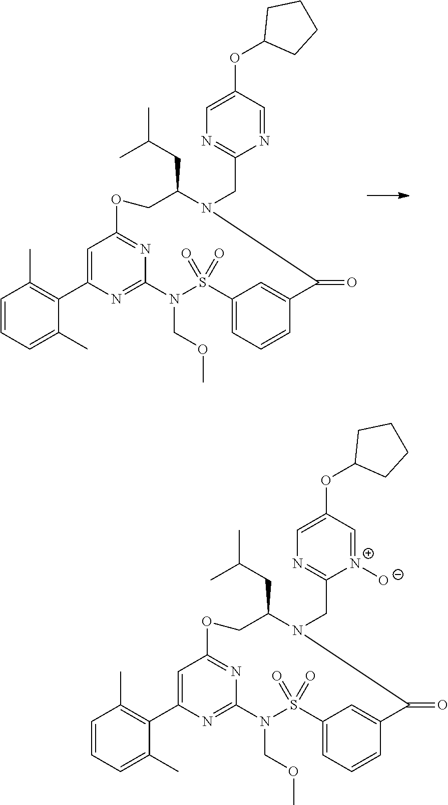 [SMILES: COCN1c2nc(cc(-c3c(C)cccc3C)n2)OC[C@@H](CC(C)C)N(Cc2ncc(OC3CCCC3)cn2)C(=O)c2cccc(c2)S1(=O)=O ; COCN1c2nc(cc(-c3c(C)cccc3C)n2)OC[C@@H](CC(C)C)N(Cc2ncc(OC3CCCC3)c[n+]2[O-])C(=O)c2cccc(c2)S1(=O)=O]

To (11R)-12-[[5-(cyclopentoxy)pyrimidin-2-yl]methyl]-6-(2,6-dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (16.9 mg, 0.02200 mmol) was added m-CPBA (6 mg, 0.02434 mmol) followed by DCM (200 μL). This was allowed to stir for 16 hours and was then partitioned between sodium bicarbonate and water. The aqueous layer was then extracted with DCM (3×) and the combined organic layer was washed with brine, dried sodium sulfate, filtered, and concentrated. This material was purified by reverse phase HPLC (1-99 MeCN in H₂O with 0.05% HCl) to give the solid (11R)-12-[[5-(cyclopentoxy)-1-oxido-pyrimidin-1-ium-2-yl]methyl]-6-(2,6-dimethylphenyl)-11-isobutyl-3-(methoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (10.3 mg, 62%) ESI-MS m/z calc. 716.2992, found 717.5 (M+1)⁺; Retention time: 2.15 minutes; LC method A.

### Step 3: (11R)-12-[[5-(Cyclopentoxy)-1-oxido-pyrimidin-1-ium-2-yl]methyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1305)

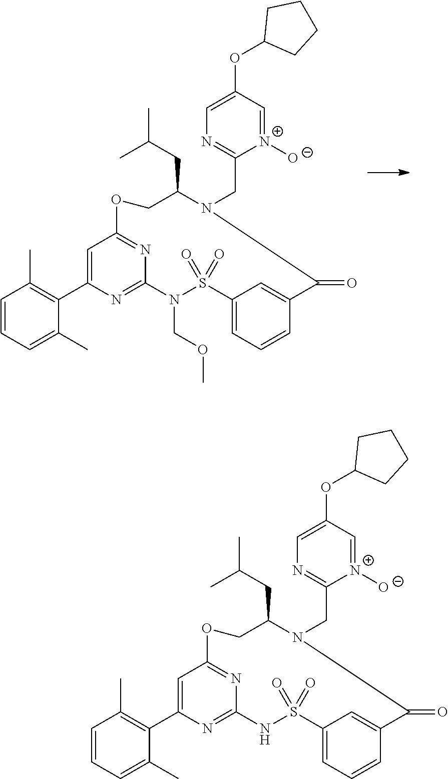 [SMILES: COCN1c2nc(cc(-c3c(C)cccc3C)n2)OC[C@@H](CC(C)C)N(Cc2ncc(OC3CCCC3)c[n+]2[O-])C(=O)c2cccc(c2)S1(=O)=O ; Cc1cccc(C)c1-c1cc2nc(n1)NS(=O)(=O)c1cccc(c1)C(=O)N(Cc1ncc(OC3CCCC3)c[n+]1[O-])[C@H](CC(C)C)CO2]

To (11R)-12-[[5-(cyclopentoxy)-1-oxido-pyrimidin-1-ium-2-yl]methyl]-6-(2,6-dimethylphenyl)-11-isobutyl-3-(methoxymethyl)-2,2-dioxo-9-oxa-2?⁶-thia-3,5,12,19-tetrazatricyclo[12.3.1.14,8]nonadeca-1(18),4(19),5,7,14,16-hexaen-13-one (hydrochloride salt) (12.3 mg, 0.01633 mmol) was added HCl (123 μL of 4 M, 0.4920 mmol) in dioxanes which was allowed to stir for 15 minutes. This was then concentrated to give the desired product (11R)-12-[[5-(cyclopentoxy)-1-oxido-pyrimidin-1-ium-2-yl]methyl]-6-(2,6-dimethylphenyl)-11-isobutyl-2,2-dioxo-9-oxa-2λ⁶-thia-3,5,12,19-tetrazatricyclo[12.3.1.14,8]nonadeca-1(18),4(19),5,7,14,16-hexaen-13-one (hydrochloride salt) (11.3 mg, 98%). ESI-MS m/z calc. 672.273, found 673.5 (M+1)⁺; Retention time: 1.73 minutes; LC method A. ¹H NMR (400 MHz, DMSO) δ 13.03 (s, 1H), 8.65 (s, 1H), 8.59 (d, J 2.4 Hz, 1H), 8.14 (d, J 2.4 Hz, 1H), 7.94 (d, J 5.7 Hz, 1H), 7.68 (d, J 4.8 Hz, 2H), 7.26 (t, J 7.6 Hz, 1H), 7.13 (d, J 7.6 Hz, 2H), 6.39 (s, 1H), 5.29 (dd, J 10.8, 4.3 Hz, 1H), 5.14-4.97 (m, 2H), 4.52 (d, J 18.0 Hz, 1H), 4.23 (q, J 11.1 Hz, 1H), 4.03 (tt, J 11.2, 3.9 Hz, 1H), 3.78-3.59 (m, 3H), 3.64-3.40 (m, 2H), 1.95 (s, 6H), 1.95 (h, J 6.8 Hz, 1H), 1.78-1.67 (m, 4H), 1.71-1.58 (m, 1H), 1.58 (dt, J 10.9, 5.3 Hz, 1H), 1.51-1.34 (m, 1H), 1.28-1.15 (m, 2H), 0.76 (d, J 6.6 Hz, 3H), 0.27 (d, J 6.4 Hz, 3H).

### Example 148: Preparation of Compound 1306

### Step 1: 2-(1-Methylcyclopropyl)ethanol

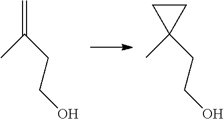

To a solution of diethylzinc (hexane solution) (2 L of 1 M, 2.0000 mol) was added 3-methylbut-3-en-1-ol (135 g, 1.5674 mol) at 0-15° C. in 30 minutes. The mixture was then warmed to 15° C. and after 20 minutes of stirring, diiodomethane (482.7 g, 1.8022 mol) as a DCM (270 mL) solution was added over 1 h. The reaction was then warmed to 25° C. and stirred for 20 hours. After cooling to 5° C., the reaction mixture was quenched with aqueous HCl (2 M, 1.35 L). The phases were split, and the aqueous phase extracted with DCM (2×675 mL). The hexanes extract was, washed with sodium thiosulfate (10% w/w, 1.35 L), and concentrated in vacuo. The sodium thiosulfate solution was then extracted with the two DCM extracts successively. The DCM extracts were then combined with the product obtained from the concentration of the hexanes extract. Water (1.35 L) was then added to this combined organic phase. The mixture was cooled to 2° C. and an aqueous solution of sodium permanganate (83.5 g, 40% w/w, 235.34 mmol) was added. The mixture was stirred at 2° C. for 15 minutes, sodium bisulfite (10% w/w, 1 L) was added, the phases were split and the aqueous phase washed with DCM (2×350 mL). The organic extracts were combined, dried over sodium sulfate, filtered and concentrated in vacuo and distilled (40° C., 2-5 mbar) to give 2-(1-methylcyclopropyl)ethanol (106.6 g, 66%) as a clear oil. ¹H NMR (400 MHz, CDCl₃) δ 3.75 (t, J 7.0 Hz, 2H), 1.51 (t, J 7.0 Hz, 3H), 1.04 (s, 3H), 0.32-0.22 (m, 4H).

### Step 2: 2-(1-Methylcyclopropyl)acetaldehyde

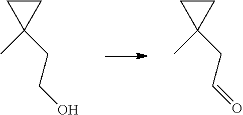

2-(1-Methylcyclopropyl)ethanol (106 g, 1.0319 mol) was added to a mixture of water (800 mL) and DCM (800 mL) before sodium bromide (10.6 g, 103.02 mmol), sodium bicarbonate (200 g, 2.3808 mol) and TEMPO (1.6 g, 10.240 mmol) were successively added. The mixture was cooled down to 0° C. and a NaOCl aqueous solution (1.3 L of 0.8 M, 1.0400 mol) was added over 1 hour (T=1.0 to 8.2° C.). After 1 hour, the mixture was filtered over celite (0.5 part) and the phases were separated. The aqueous phase was extracted with DCM (2×3.5 vol). The combined organic phases were dried with sodium sulfate (0.5 part) and concentrated under reduced pressure (300 mbar, bath: 30° C.) to give a slightly amber DCM solution with a content of 4.15% w/w of 2-(1-methylcyclopropyl)acetaldehyde (101.27 g, 100%). ¹H NMR (400 MHz, Chloroform-d) δ 9.85-9.79 (m, 1H), 2.26 (d, J 2.4 Hz, 2H), 1.13 (s, 3H), 0.45 (br d, J=6.4 Hz, 4H).

### Step 3: 3-(1-Methylcyclopropyl)-2-[[(1R)-1-phenylethyl]amino]propanenitrile

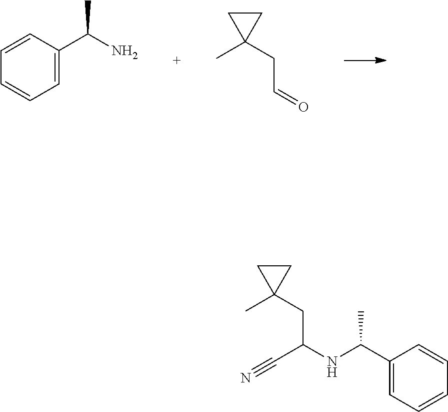

To a solution of 2-(1-methylcyclopropyl)acetaldehyde (101.27 g, 1.0319 mol) in MeOH (850 mL), cooled to 0° C. in an ice bath, was added portionwise (1R)-1-phenylethanamine (122.20 g, 130 mL, 1.0084 mol) (T increased from 3° C. to 9° C.). Acetic acid (68.640 g, 65 mL, 1.1430 mol) was added dropwise (T increased from 3° C. to 5° C.) followed by the portionwise addition of sodium cyanide (53 g, 1.0815 mol) (T increased from 1° C. to 5° C.). The mixture was allowed to warm to room temperature and stirred overnight. The mixture was concentrated under vacuum (rotovapor connected to a scrubber containing a 6 M aqueous solution of sodium hydroxide). To the residue, MTBE (5 vol.) and an aqueous solution of potassium carbonate (10% w/w, 5 vol) were added. The mixture was stirred 5 min before the phases were split. The organic layer was washed with brine (15% w/w, 3×5 vol.), dried with sodium sulfate (0.5 part) and concentrated under reduced pressure to afford 3-(1-methylcyclopropyl)-2-[[(1R)-1-phenylethyl]amino]propanenitrile (diastereomeric mixture 70:30, 245.86 g, 92%) as a slightly amber oil. ESI-MS m/z calc. 228.16264, found 229.2 (M+1)⁺; Retention time: 2.935 minutes; LC method U.

### Step 4: (2R)-3-(1-Methylcyclopropyl)-2-[[(1R)-1-phenylethyl]amino]propanamide

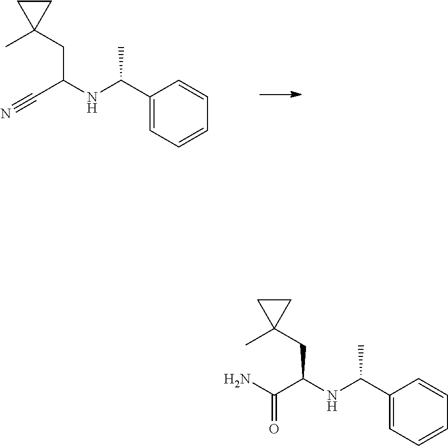 [SMILES: C[C@@H](NC(C#N)CC1(C)CC1)c1ccccc1 ; C[C@@H](N[C@H](CC1(C)CC1)C(N)=O)c1ccccc1]

To a solution combining two batches of 3-(1-methylcyclopropyl)-2-[[(1R)-1-phenylethyl]amino]propanenitrile (diastereomeric mixture 70:30, 245 g, 948.53 mmol, and 19 g, 68.899 mmol) in a mixture of DMSO (1.2 L) and water (250 mL) stirred at 50° C., was added potassium carbonate (33 g, 238.77 mmol). An aqueous solution of hydrogen peroxide (220 mL of 9.8 M, 2.1560 mol) was added dropwise over 1.5 hours. The mixture was stirred for 1 hour at 50° C. The mixture was cooled down to room temperature before water (5 L, 20 vol) was added. The aqueous layer was extracted with MTBE (2×1.5 L, 2×6 vol). The combined organic layers were isolated and extracted with an aqueous solution of HCl (2×1.5 L of 1 M, 2×6 vol). The acidic aqueous layers were combined and slowly stirred at 5° C. for 2 hours. The resulting suspension was filtered and the solids recovered were dried under reduced pressure at 50° C. for 2 hours to afford (2R)-3-(1-methylcyclopropyl)-2-[[(1R)-1-phenylethyl]amino]propanamide (hydrochloride salt) (151.24 g, 52%) as a white powder. ESI-MS m/z calc. 246.1732, found 274.2 (M+1)⁺; Retention time: 1.391 minutes. ¹H NMR (400 MHz, DMSO-d₆) δ 10.04-9.89 (m, 1H), 9.56-9.37 (m, 1H), 8.05 (s, 1H), 7.77-7.56 (m, 3H), 7.47-7.37 (m, 3H), 4.28-4.14 (m, 1H), 3.45-3.28 (m, 1H), 2.20-2.07 (m, 1H), 1.59 (d, J 6.8 Hz, 3H), 1.44 (dd, J 13.4, 10.8 Hz, 1H), 0.92 (s, 3H), 0.35-0.11 (m, 4H); LC method U.

To the mother liquor was added sodium chloride (500 g, 2 parts) and the aqueous solution was stirred at 5° C. overnight. The resulting suspension was filtered and the solids recovered were dried with an air flow for 2 hours to afford 3-(1-methylcyclopropyl)-2-[[(1R)-1-phenylethyl]amino]propanamide (hydrochloride salt) (89.42 g, 31%) as a white powder isomeric mixture. ESI-MS m/z calc. 246.1732, found 247.2 (M+1)⁺; Retention time: 1.541 minutes; LC method U.

### Step 5: (2R)-3-(1-Methylcyclopropyl)-2-[[(1R)-1-phenylethyl]amino]propanoic

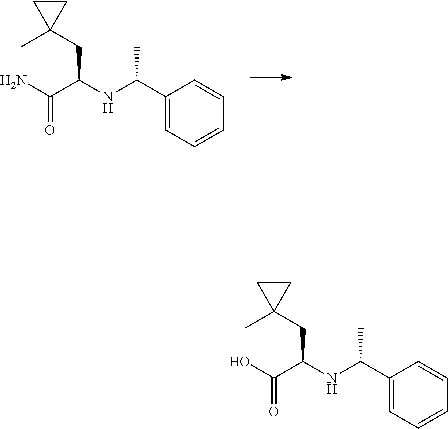 [SMILES: C[C@@H](N[C@H](CC1(C)CC1)C(N)=O)c1ccccc1 ; C[C@@H](N[C@H](CC1(C)CC1)C(=O)O)c1ccccc1]

To a solution of lithium hydroxide monohydrate (324 g, 7.7210 mol) in water (5 L) was added at 70° C. (2R)-3-(1-methylcyclopropyl)-2-[[(1R)-1-phenylethyl]amino]propanamide (hydrochloride salt) (228.9 g, 809.38 mmol). The reaction was heated to 97° C. and stirred for 68 hours. The reaction mixture was then cooled to room temperature and neutralized to pH 6 using 3M aqueous HCl and the product recovered by filtration. The product was then recrystallized four times by dissolving it in 0.5M aqueous NaOH (5 L) and neutralizing to pH 6 using 3M aqueous HCl. The product obtained was then dried in vacuo at 45° C. for 72 h and (2R)-3-(1-methylcyclopropyl)-2-[[(1R)-1-phenylethyl]amino]propanoic acid (161.5 g, 65%) was obtained as a white solid. ¹H NMR (400 MHz, DMSO-d₆) δ 7.62 (d, J 6.4 Hz, 2H), 7.45-7.35 (m, 3H), 4.31 (q, J 6.4 Hz, 1H), 3.25 (dd, J 10.9, 3.8 Hz, 1H), 2.34 (dd, J 13.6, 3.5 Hz, 1H), 1.61 (d, J 6.6 Hz, 3H), 1.33 (dd, J 13.3, 11.4 Hz, 1H), 0.82 (s, 3H), 0.25-0.11 (m, 4H). 2H labile missing. ESI-MS m/z calc. 247.15723, found 248.2 (M+1)⁺; Retention time: 1.68 minutes; LC method U.

### Step 6: (2R)-3-(1-Methylcyclopropyl)-2-[[(1R)-1-phenylethyl]amino]propan-1-ol

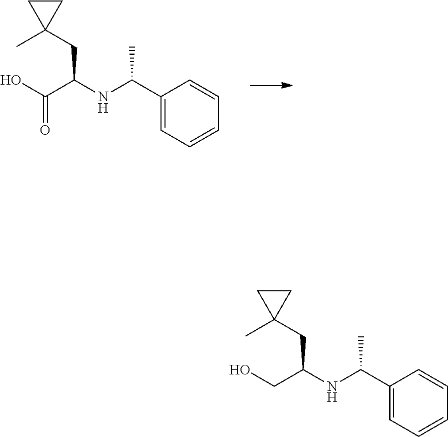 [SMILES: C[C@@H](N[C@H](CC1(C)CC1)C(=O)O)c1ccccc1 ; C[C@@H](N[C@@H](CO)CC1(C)CC1)c1ccccc1]

To a solution of (2R)-3-(1-methylcyclopropyl)-2-[[(1R)-1-phenylethyl]ammonio]propanoate (160 g, 523.26 mmol) in THF (3.2 L) was added LiAlH₄ (40 g, 1.0539 mol) over 3 hours at 20-25° C. After an additional hour of stirring at room temperature, the reaction mixture was cooled down to 10° C. and water (38.000 g, 38 mL, 2.1093 mol) was added over 150 minutes. NaOH (35 mL of 6 M, 210.00 mmol) and water (38.000 g, 38 mL, 2.1093 mol) were then successively added. The mixture was stirred overnight, at room temperature. The reaction mixture was then filtered over a bed of Celite (bottom, 80 g) and magnesium sulfate (top, 120 g). The cake was washed with THF (800 mL). The combined mother liquors were concentrated in vacuo. The resulting yellowish oil was dissolved in diethyl ether (2 L) and a solution of HCl in dioxane (130 mL of 4 M, 520.00 mmol) was added dropwise over 30 minutes inducing a precipitation. After 1 hour of stirring at 20° C., the solids were recovered by filtration, washed with diethyl ether (1 L) and dried in vacuo to afford (2R)-3-(1-methylcyclopropyl)-2-[[(1R)-1-phenylethyl]amino]propan-1-ol (hydrochloride salt) (126 g, 89%)¹H NMR (400 MHz, DMSO-d₆) δ 9.30 (br. s., 1H), 9.11 (br. s., 1H), 7.69 (d, J 7.1 Hz, 2H), 7.48-7.35 (m, 3H), 5.43 (t, J 5.3 Hz, 1H), 4.56 (br. s., 1H), 3.79 (d, J 12.5 Hz, 1H), 3.65-3.52 (m, 1H), 2.89 (br. s., 1H), 1.82 (dd, J 13.7, 2.2 Hz, 1H), 1.62 (d, J 6.6 Hz, 3H), 1.26 (dd, J 13.7, 11.2 Hz, 1H), 0.72 (s, 3H), 0.41-0.27 (m, 1H), 0.24-0.06 (m, 3H). ESI-MS m/z calc. 233.17796, found 234.2 (M+1)⁺; Retention time: 1.82 minutes; LC method U.

### Step 7: (2R)-2-Amino-3-(1-methylcyclopropyl)propan-1-ol

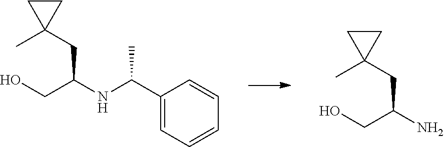

A solution of (2R)-3-(1-methylcyclopropyl)-2-[[(1R)-1-phenylethyl]amino]propan-1-ol (hydrochloride salt) (125 g, 463.29 mmol) in ethanol (1.5 L) was added to palladium on carbon (25 g, 5% w/w, 11.746 mmol). The reaction vessel was purged with nitrogen and then filled with hydrogen (75 psi) and the reaction was stirred at 50° C. for 24 h. The mixture was filtered through a Pall filter 0.45 um, washing with EtOH (2×500 mL), and the filtrate concentrated in vacuo. The white solid obtained was triturated with MTBE (625 mL) during 1 hour and then filtered, washed with MTBE (500 mL) and dried in vacuo to afford (2R)-2-amino-3-(1-methylcyclopropyl)propan-1-ol (hydrochloride salt) (71.78 g, 93%) as a white solid. ¹H NMR (400 MHz, DMSO-d₆) δ 7.98 (br. s., 3H), 5.30 (t, J 4.9 Hz, 1H), 3.69 (dt, J 11.4, 3.6 Hz, 1H), 3.47 (dt, J 11.5, 5.7 Hz, 1H), 3.29-3.16 (m, 1H), 1.62 (dd, J 13.9, 5.9 Hz, 1H), 1.35 (dd, J=13.9, 8.6 Hz, 1H), 1.01 (s, 3H), 0.41-0.19 (m, 4H). ESI-MS m/z calc. 129.11537, found 130.2 (M+1)⁺; Retention time: 0.34 minutes; LC method U.

### Step 8: (2R)-2-[(5-Isopropoxypyrimidin-2-yl)methylamino]-3-(1-methylcyclopropyl)propan-1-ol

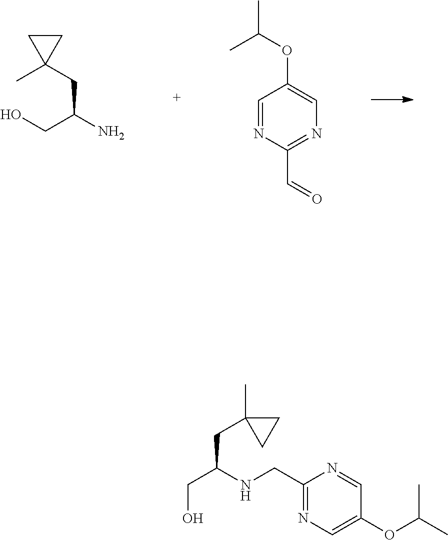

A 500 mL flask was charged under nitrogen with (2R)-2-amino-3-(1-methylcyclopropyl)propan-1-ol (hydrochloride salt) (3.38 g, 19.38 mmol) and 1,2-dichloroethane (70 mL). To the suspension was added DIEA (3.7 mL, 21.24 mmol) and the mixture was stirred for 10 minutes at room temperature. To the suspension was added 5-isopropoxypyrimidine-2-carbaldehyde (3.23 g, 19.44 mmol), resulting in immediate dissolution of the solids. After adding acetic acid (1.2 mL, 21.10 mmol), the mixture was stirred at room temperature for 2 h. To the mixture was added sodium triacetoxyborohydride (5.38 g, 25.38 mmol) in three equal portions over 10 minutes and the mixture was continued to stir at room temperature for an additional 3 hours. The reaction mixture was cooled in an ice water bath and slowly quenched with aqueous NaOH (100 mL of 2 M, 200.0 mmol) and brine (30 mL). The reaction mixture was transferred to a separatory funnel, the layers were separated, and the aqueous layer was extracted with diethyl ether (3×30 mL). The combined organic phases were washed with brine (40 mL) and dried over sodium sulfate, filtered, and concentrated under reduced pressure. The crude material was dissolved in DCM and purified by flash chromatography on silica gel (220 g column) using a gradient of methanol (containing 10% of concentrated aqueous ammonium hydroxide, 0 to 10% over 30 min gradient) in dichloromethane to give (2R)-2-[(5-isopropoxypyrimidin-2-yl)methylamino]-3-(1-methylcyclopropyl)propan-1-ol (4.47 g, 74%) as a tan oil that solidified upon drying. ¹H NMR (400 MHz, Chloroform-d) δ 8.34 (s, 2H), 4.65-4.53 (m, 1H), 4.17-4.01 (m, 2H), 3.74 (dd, J=11.0, 3.5 Hz, 1H), 3.40 (dd, J=11.0, 5.5 Hz, 1H), 2.99-2.87 (m, 1H), 1.38 (d, J 6.1 Hz, 6H), 1.35 (dt, J 6.5, 1.9 Hz, 2H), 1.02 (s, 3H), 0.40-0.20 (m, 4H). ESI-MS m/z calc. 279.19467, found 280.66 (M+1)⁺; Retention time: 0.83 minutes; LC method A.

### Step 9: 3-[[4-(2,6-Dimethylphenyl)-6-[(2R)-2-[(5-isopropoxypyrimidin-2-yl)methylamino]-3-(1-methylcyclopropyl)propoxy]pyrimidin-2-yl]sulfamoyl]benzoic acid

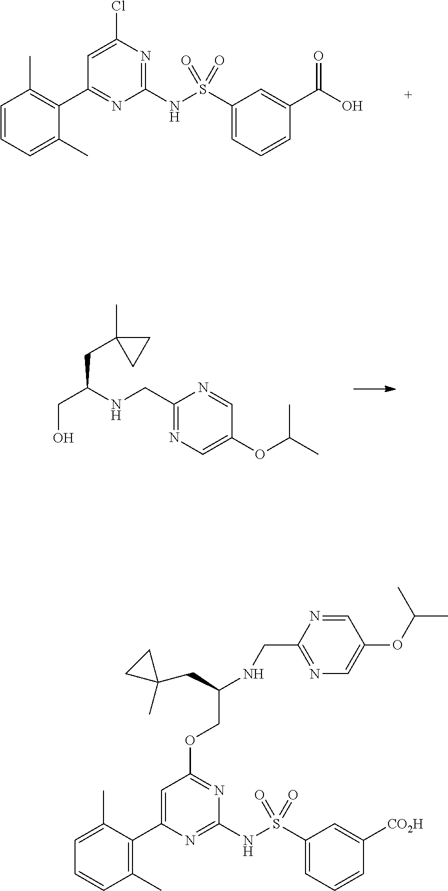 [SMILES: Cc1cccc(C)c1-c1cc(Cl)nc(NS(=O)(=O)c2cccc(C(=O)O)c2)n1 ; CC(C)Oc1cnc(CN[C@@H](CO)CC2(C)CC2)nc1 ; Cc1cccc(C)c1-c1cc(OC[C@@H](CC2(C)CC2)NCc2ncc(OC(C)C)cn2)nc(NS(=O)(=O)c2cccc(C(=O)O)c2)n1]

A 250 mL flask was charged under nitrogen with (2R)-2-[(5-isopropoxypyrimidin-2-yl)methylamino]-3-(1-methylcyclopropyl)propan-1-ol (4.45 g, 14.34 mmol), 3-[[4-chloro-6-(2,6-dimethylphenyl)pyrimidin-2-yl]sulfamoyl]benzoic acid (6.02 g, 14.41 mmol) and anhydrous THF (100 mL). The mixture was stirred for about 10 min until all solids dissolved. The mixture was then stirred in an ice-water bath until internal temperature reached 5° C. Sodium tert-butoxide (6.85 g, 71.28 mmol) was added in two portions raising internal temperature to 12° C., at which time the ice bath was removed, and the reaction was stirred without external cooling for 1.5 h. More sodium tert-butoxide (1.19 g, 12.38 mmol) was added and the mixture was stirred for one hour. The reaction mixture was cooled in an ice-water bath and then quenched with an aqueous HCl (30 mL of 6 M, 180.0 mmol) solution, brine (40 mL) and 1N aqueous HCl (40 mL). The reaction mixture was transferred to a separatory funnel. The layers were separated, and the aqueous layer was extracted once with EtOAc (40 mL). The combined organic phases were washed with brine (40 mL) dried over sodium sulfate, filtered, and concentrated to an orange resin. The material was dissolved in DCM and purified by flash chromatography on silica gel (330 g column) using a gradient of methanol (0 to 15% over 30 min) in dichloromethane. After evaporation, the residue was triturated with DCM/hexanes and the solvents were evaporated. The operation was repeated twice. After drying under vacuum, 3-[[4-(2,6-dimethylphenyl)-6-[(2R)-2-[(5-isopropoxypyrimidin-2-yl)methylamino]-3-(1-methylcyclopropyl)propoxy]pyrimidin-2-yl]sulfamoyl]benzoic acid (hydrochloride salt) (7.11 g, 71%) was isolated as a tan solid. ¹H NMR (400 MHz, DMSO-d₆) δ 8.56 (s, 2H), 8.47 (t, J 1.8 Hz, 1H), 8.13 (dt, J 7.9, 1.7 Hz, 2H), 7.69 (t, J 7.8 Hz, 1H), 7.25 (t, J 7.6 Hz, 1H), 7.12 (d, J 7.6 Hz, 2H), 6.29 (s, 1H), 4.82 (hept, J 6.0 Hz, 1H), 4.55 (d, J 12.1 Hz, 1H), 4.33 (d, J 17.5 Hz, 3H), 3.69 (s, 1H), 2.00 (s, 6H), 1.48-1.37 (m, 1H), 1.29 (d, J 6.0 Hz, 6H), 1.26-1.22 (m, 1H), 1.01 (s, 3H), 0.38-0.30 (m, 1H), 0.30-0.23 (m, 1H), 0.23-0.13 (m, 2H). ESI-MS m/z calc. 660.273, found 661.71 (M+1)⁺; Retention time: 1.25 minutes; LC method A.

### Step 10: (11R)-6-(2,6-Dimethylphenyl)-12-[(5-isopropoxypyrimidin-2-yl)methyl]-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1306)

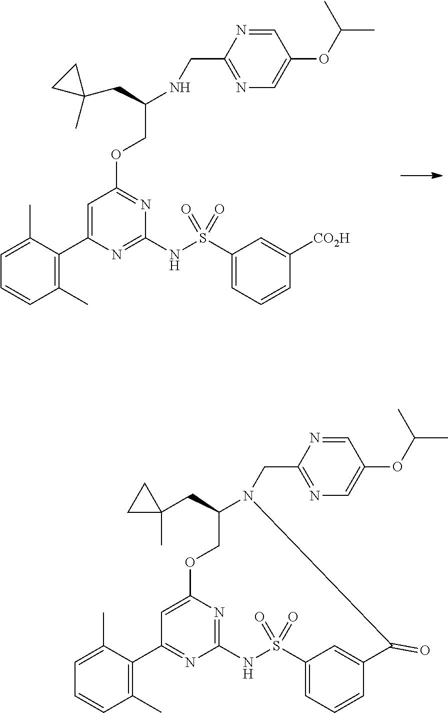 [SMILES: Cc1cccc(C)c1-c1cc(OC[C@@H](CC2(C)CC2)NCc2ncc(OC(C)C)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ncc(OC(C)C)cn1)[C@H](CC1(C)CC1)CO2]

3-[[4-(2,6-Dimethylphenyl)-6-[(2R)-2-[(5-isopropoxypyrimidin-2-yl)methylamino]-3-(1-methylcyclopropyl)propoxy]pyrimidin-2-yl]sulfamoyl]benzoic acid (hydrochloride salt) (7.07 g, 10.14 mmol) was combined in a 500 mL flask under nitrogen with CDMT (2.713 g, 15.45 mmol) and anhydrous DMF (300 mL). The solution was stirred in an ice-water bath until internal temperature reached 4° C., at which time 4-Methyl-morpholine (5.5 mL, 50.03 mmol) was added through syringe over 2 minutes. The solution was stirred for 4 hours in the cooling bath that was allowed to warm to room temperature (at which time internal temperature had reached 18° C.). The mixture was poured into a separatory funnel with ethyl acetate (500 mL), brine (100 mL) and water (100 mL). The funnel was shaken, and the phases separated and the aqueous layer was extracted with ethyl acetate (2×100 mL). The combined extracts were washed with brine (100 mL), then a mixture of water (100 mL) and brine (50 mL). The last three wash solutions were combined and back extracted with EtOAc (100 mL). The combined organic phases were dried over sodium sulfate and the solvents were concentrated to a crude solid (11.5 g). The material was dissolved in a minimum volume of DCM containing a bit of methanol and purified by flash chromatography on silica gel (220 g column) using a gradient of ethyl acetate (5 to 100% over 30 min) in hexanes to give 5.26 g of impure material. This material was dissolved in a mixture of DMSO (25 mL) and MeOH (20 mL) and purified by reverse phase HPLC (50×100 mm C₁₈, 10 μM column, 100 mL/min flow rate, 11×5 mL injections, gradient 10-99% acetonitrile/5 mM HCl over 15 min). The pure fractions were combined, brine (100 mL) was added, and the acetonitrile was evaporated. The solid in suspension was extracted twice with EtOAc (150 mL and 50 mL). The combined organic phases were dried over sodium sulfate and concentrated. The solid was slurried in a mixture of EtOAc and hexanes (1:3, v:v), filtered and dried in a vacuum oven at 40° C. over 2 days to give (11R)-6-(2,6-dimethylphenyl)-12-[(5-isopropoxypyrimidin-2-yl)methyl]-11-[(1-methylcyclopropyl)methyl]-2,2-dioxo-9-oxa-2?⁶-thia-3,5,12,19-tetrazatricyclo[12.3.1.14,8]nonadeca-1(18),4(19),5,7,14,16-hexaen-13-one (3.52 g, 53%) as a white solid. ¹H NMR (400 MHz, DMSO-d₆) δ 13.71-11.37 (Broad m, 1H), 8.65 (s, 1H), 8.52 (s, 2H), 7.93 (s, 1H), 7.67 (s, 2H), 7.31-7.19 (m, 1H), 7.12 (d, J=7.6 Hz, 2H), 6.40 (s, 1H), 5.35 (d, J=9.8 Hz, 1H), 4.88-4.74 (m, 2H), 4.52 (d, J=16.6 Hz, 1H), 4.32-4.11 (m, 2H), 2.20-1.88 (m, 6H), 1.61 (d, J 14.8 Hz, 1H), 1.47 (dd, J 15.1, 9.4 Hz, 1H), 1.30 (dd, J 6.0, 2.4 Hz, 6H), 0.37 (s, 3H), 0.27 (dt, J 9.5, 4.7 Hz, 1H), 0.16 (dt, J 9.5, 4.7 Hz, 1H), 0.06 (d, J 4.5 Hz, 1H), −0.02-−0.10 (overlapped with TMS, m, 1H). ESI-MS m/z calc. 642.26245, found 643.66 (M+1)⁺; Retention time: 1.8 minutes; LC method A.

### Example 149: Preparation of Compound 1307

### Step 1: 3-[[4-[(2R)-2-[[5-(3,3-Dimethylazetidin-1-yl)pyrimidin-2-yl]methylamino]-3-(1-methylcyclopropyl)propoxy]-6-(2,6-dimethylphenyl)pyrimidin-2-yl]sulfamoyl]benzoic acid

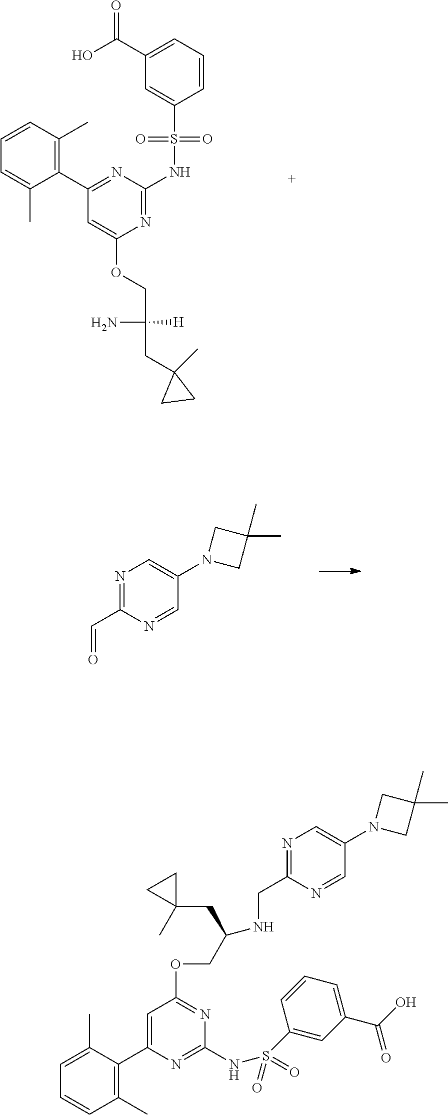 [SMILES: Cc1cccc(C)c1-c1cc(OC[C@H](N)CC2(C)CC2)nc(NS(=O)(=O)c2cccc(C(=O)O)c2)n1 ; CC1(C)CN(c2cnc(C=O)nc2)C1 ; Cc1cccc(C)c1-c1cc(OC[C@@H](CC2(C)CC2)NCc2ncc(N3CC(C)(C)C3)cn2)nc(NS(=O)(=O)c2cccc(C(=O)O)c2)n1]

To a stirred mixture of 3-[[4-[(2R)-2-amino-3-(1-methylcyclopropyl)propoxy]-6-(2,6-dimethylphenyl)pyrimidin-2-yl]sulfamoyl]benzoic acid (hydrochloride salt) (84 mg, 0.1535 mmol) and 5-(3,3-dimethylazetidin-1-yl)pyrimidine-2-carbaldehyde (30 mg, 0.1569 mmol) in anhydrous dichloromethane (0.7 mL) were added glacial acetic acid (10 μL, 0.1758 mmol) and N,N-diisopropylethylamine (50 μL, 0.2871 mmol), in that order, at 0° C. (ice-water bath). The yellow solution was stirred for 2-3 min, then sodium triacetoxyborohydride (100 mg, 0.4718 mmol) was added at once at the same temperature. After stirring for another 6 min, the reaction mixture was poured over ice-water (20 mL) containing HCl. The product was extracted with ethyl acetate (3×30 mL) and the combined organics were washed with water (20 mL), brine (20 mL), dried over sodium sulfate, filtered and concentrated under reduced pressure. The crude was purified by silica gel chromatography (40 g gold column, 1-15% methanol in dichloromethane over 15 min, peak came around 8% methanol) to furnish desired 3-[[4-[(2R)-2-[[5-(3,3-dimethylazetidin-1-yl)pyrimidin-2-yl]methylamino]-3-(1-methylcyclopropyl)propoxy]-6-(2,6-dimethylphenyl)pyrimidin-2-yl]sulfamoyl]benzoic acid (83 mg, 79%) as an off-white solid. It was used in the cyclization step. ESI-MS m/z calc. 685.3046, found 686.3 (M+1)⁺; Retention time: 1.39 minutes; LC method A.

### Step 2: (11R)-12-[[5-(3,3-Dimethylazetidin-1-yl)pyrimidin-2-yl]methyl]-6-(2,6-dimethylphenyl)-11-[(1-methylcyclopropyl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1307)

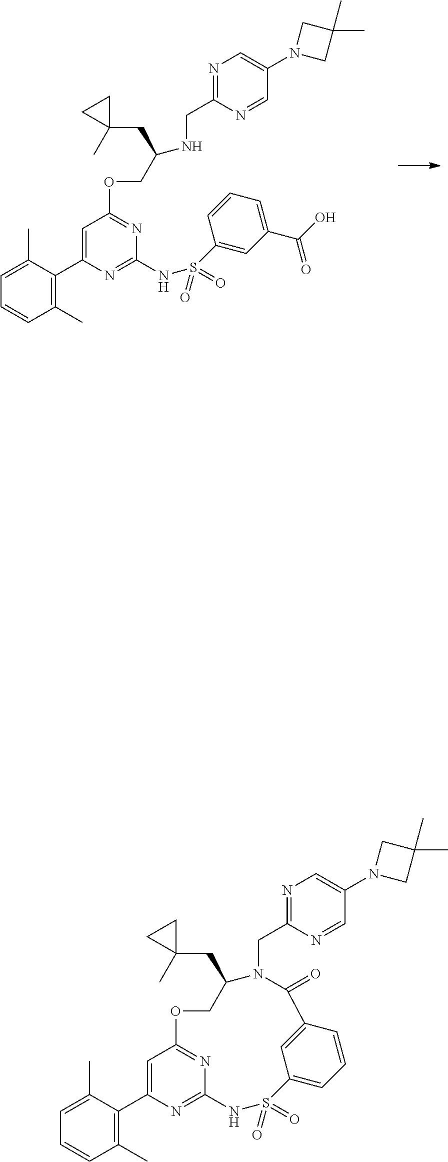 [SMILES: Cc1cccc(C)c1-c1cc(OC[C@@H](CC2(C)CC2)NCc2ncc(N3CC(C)(C)C3)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ncc(N3CC(C)(C)C3)cn1)[C@H](CC1(C)CC1)CO2]

To a stirred solution of 3-[[4-[(2R)-2-[[5-(3,3-dimethylazetidin-1-yl)pyrimidin-2-yl]methylamino]-3-(1-methylcyclopropyl)propoxy]-6-(2,6-dimethylphenyl)pyrimidin-2-yl]sulfamoyl]benzoic acid (83 mg, 0.1210 mmol) in anhydrous N,N-dimethylformamide (5 mL) was added 2-chloro-4,6-dimethoxy-1,3,5-triazine (60 mg, 0.3417 mmol) (CDMT), followed by the addition of 4-methylmorpholine (100 μL, 0.9096 mmol) at 0-4° C. (ice-water bath) under nitrogen. The yellow reaction was allowed to stir at that temperature for 5 min, then allowed to stir at room temperature for 3 h. After removing most of the volatiles under reduced pressure, the residue was purified by preparative reverse-phase HPLC (1-99% acetonitrile in water over 15 min, 5 mM HCl as modifier) and the combined fractions were extracted with ethyl acetate (3×15 mL). The combined organics were washed with brine (15 mL), dried over sodium sulfate, filtered and concentrated under reduced pressure and dried under vacuum to furnish (11R)-12-[[5-(3,3-dimethylazetidin-1-yl)pyrimidin-2-yl]methyl]-6-(2,6-dimethylphenyl)-11-[(1-methylcyclopropyl)methyl]-2,2-dioxo-9-oxa-2?⁶-thia-3,5,12,19-tetrazatricyclo[12.3.1.14,8]nonadeca-1(18),4(19),5,7,14,16-hexaen-13-one (53 mg, 65%) as a white solid. ¹H NMR (400 MHz, DMSO-d₆) δ 13.05 (s, 1H), 8.65 (s, 1H), 8.03 (s, 2H), 7.97-7.84 (m, 1H), 7.67 (s, 2H), 7.25 (t, J 7.7 Hz, 1H), 7.12 (d, J 7.7 Hz, 2H), 6.39 (s, 1H), 5.40-5.28 (m, 1H), 4.81 (d, J 16.3 Hz, 1H), 4.43 (d, J 16.4 Hz, 1H), 4.21 (t, J 10.4 Hz, 1H), 4.11 (t, J 11.1 Hz, 1H), 3.64 (s, 4H), 1.96 (s, 6H), 1.60 (d, J 14.8 Hz, 1H), 1.47 (dd, J 15.2, 9.8 Hz, 1H), 1.29 (s, 6H), 0.37 (s, 3H), 0.27 (dt, J 9.6, 4.5 Hz, 1H), 0.16 (dt, J 9.3, 4.7 Hz, 1H), 0.05 (dt, J 9.3, 4.6 Hz, 1H), −0.01-−0.10 (m, 1H). ESI-MS m/z calc. 667.29407, found 668.2 (M+1)⁺; Retention time: 1.88 minutes; LC method A.

### Example 150: Preparation of Compound 1308

### Step 1: Benzyl (3R)-3-(tert-butoxycarbonylamino)-4-hydroxy-butanoate

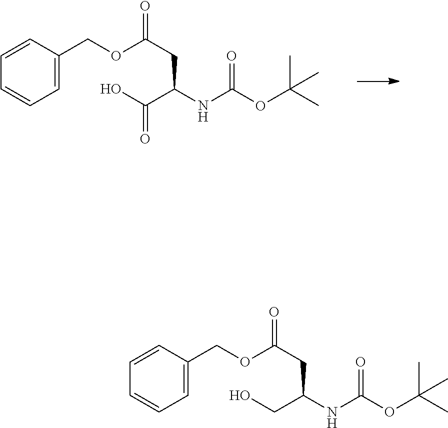 [SMILES: CC(C)(C)OC(=O)N[C@H](CC(=O)OCc1ccccc1)C(=O)O ; CC(C)(C)OC(=O)N[C@@H](CO)CC(=O)OCc1ccccc1]

A stirred solution of (2R)-4-benzyloxy-2-(tert-butoxycarbonylamino)-4-oxo-butanoic acid (20 g, 61.854 mmol) was dissolved in tetrahydrofuran (200 mL) then cooled down to −50° C. Then N-methylmorpholine (7.5440 g, 8.2 mL, 74.585 mmol) was added, followed by isobutylchloroformate (10.185 g, 9.7 mL, 74.573 mmol). The reaction was stirred at −50° C. for 2 h then the reaction was filtered, and the filtrate was cooled down to −10° C. Sodium borohydride (3.50 g, 92.513 mmol) was added and the reaction was allowed to reach room temperature and stirred at room temperature for 4 h. The reaction was quenched at 0° C. by the dropwise addition of water (200 mL). The layers were separated, and the aqueous layer was extracted with ethyl acetate (5×150 mL). The combined organic layers were washed with brine (250 mL), dried over magnesium sulfate, filtered and concentrated under reduced pressure to afford crude benzyl (3R)-3-(tert-butoxycarbonylamino)-4-hydroxy-butanoate (17.857 g, 49%) as a thick translucent oil which was used in the next step without further purification. ESI-MS m/z calc. 309.1576, found 332.2 (M+23)⁺;210.2 (M-99)⁺; Retention time: 1.7 minutes; LC method X.

### Step 2: Benzyl 2-[(4R)-2-oxooxazolidin-4-yl]acetate

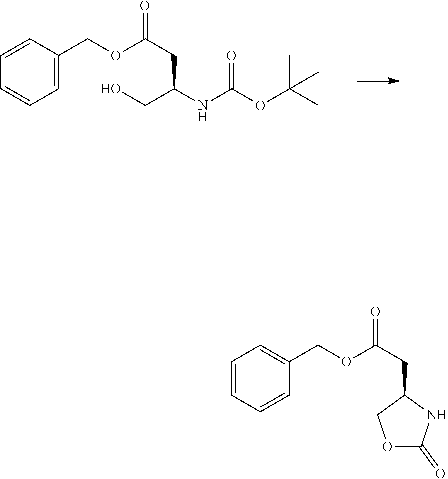 [SMILES: CC(C)(C)OC(=O)N[C@@H](CO)CC(=O)OCc1ccccc1 ; O=C(C[C@@H]1COC(=O)N1)OCc1ccccc1]

To a stirred solution of crude benzyl (3R)-3-(tert-butoxycarbonylamino)-4-hydroxy-butanoate (17.9 g, 30.667 mmol) in anhydrous 1,2-dichloroethane (140 mL) under nitrogen atmosphere at 0° C. were successively added pyridine (23.472 g, 24 mL, 296.74 mmol) and methanesulfonic anhydride (10 g, 57.407 mmol). The reaction was stirred at 0° C. for 15 minutes then stirred at room temperature for 2 h and finally stirred overnight at 90° C. The reaction was then cooled down to room temperature, diluted with dichloromethane (140 mL) and quenched by the addition of an aqueous solution of 1N hydrochloric acid (400 mL). The layers were separated, and the aqueous layer was extracted with dichloromethane (4×100 mL). the combined organic layers were washed with brine (250 mL), dried over magnesium sulfate, filtered, and concentrated under reduced pressure to afford a yellow oil which was purified by flash chromatography on silica gel using a 120 g HP Gold column and eluting with a gradient of ethyl acetate in heptanes (15 to 100% in 15 CV). The desired fractions were concentrated under reduced pressure, dried under vacuum to afford benzyl 2-[(4R)-2-oxooxazolidin-4-yl]acetate (5.01 g, 68%) as an off-white powder. ¹H NMR (400 MHz, CDCl₃) δ 7.48-7.30 (m, 5H), 5.49 (br. s, 1H), 5.16 (s, 2H), 4.56 (t, J 8.6 Hz, 1H), 4.32-4.18 (m, 1H), 4.06 (dd, J 8.9, 5.7 Hz, 1H), 2.78-2.62 (m, 2H). ESI-MS m/z calc. 235.08446, found 236.2 (M+1)⁺; Retention time: 1.51 minutes; LC method X.

### Step 3: (4R)-4-(2-Hydroxy-2-methyl-propyl)oxazolidin-2-one

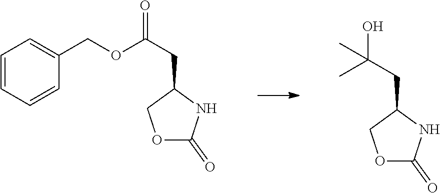 [SMILES: O=C(C[C@@H]1COC(=O)N1)OCc1ccccc1 ; CC(C)(O)C[C@@H]1COC(=O)N1]

A flask was flame-dried then cooled down to room temperature with a nitrogen stream, then charged with anhydrous toluene (30 mL) and anhydrous tetrahydrofuran (30 mL). The solvent mixture was then cooled down to −50° C. A solution of methyl magnesium bromide in diethylether (29 mL of 3 M, 87.000 mmol) was canulated to the mixture and stirred for 30 minutes at −50° C. after which time a solution of benzyl 2-[(4R)-2-oxooxazolidin-4-yl]acetate (5.01 g, 18.678 mmol) in anhydrous tetrahydrofuran (15 mL) was canulated. The reaction was stirred for 30 minutes at −50° C. then allowed to reach room temperature and stirred overnight at room temperature. The reaction was then cooled down to 0° C. and quenched by the dropwise addition of a solution of acetic acid (7.9200 g, 7.5 mL, 131.89 mmol) in water (20 mL). The reaction mixture was vigorously stirred at room temperature for 1 h. Then sodium chloride was added to saturate the aqueous layer. The reaction was then dried over sodium sulfate and filtered on celite pad. The cake was washed with dichloromethane (5×100 mL) and the filtrate was concentrated under reduced pressure to afford a yellow oil which was purified by flash chromatography on silica gel using a 120 g HP Gold column and eluting with a gradient of isopropanol in dichloromethane (0 to 6% in 20 CV). The desired fractions were concentrated under reduced pressure, dried under high vacuum to afford (4R)-4-(2-hydroxy-2-methyl-propyl)oxazolidin-2-one (2.02 g, 65%) as pale yellow crystalline solid. ¹H NMR (400 MHz, DMSO-d₆) δ 7.34 (br. s, 1H), 4.50-4.26 (m, 2H), 4.06-3.88 (m, 2H), 1.78-1.48 (m, 2H), 1.10 (s, 6H). ESI-MS m/z calc. 159.08954, found 160.2 (M+1)⁺; Retention time: 0.74 minutes; LC method X.

### Step 4: (4R)-4-(2-Fluoro-2-methyl-propyl)oxazolidin-2-one

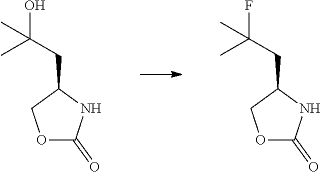

To a stirred solution of (diethylamino)sulfur trifluoride (4.1480 g, 3.4 mL, 25.734 mmol) in anhydrous dichloromethane (70 mL) at −78° C. was canulated a solution of (4R)-4-(2-hydroxy-2-methyl-propyl)oxazolidin-2-one (4.29 g, 25.603 mmol) in anhydrous dichloromethane (25 mL). The resulting solution was stirred for 15 min at −78° C. then allowed to reach room temperature and stirred at room temperature for 2 h. The reaction was then slowly added to a solution of saturated aqueous sodium bicarbonate (500 mL) at 0° C. The solution was then vigorously stirred for 30 minutes at room temperature. The layers were separated, and the aqueous layer was extracted with dichloromethane (4×150 mL). The combined organic layers were washed with water (200 mL), brine (200 mL), dried over magnesium sulfate, filtered and concentrated under reduced pressure to afford crude (4R)-4-(2-fluoro-2-methyl-propyl)oxazolidin-2-one (3.51 g, 81%) as brown crystals which were directly used in the next step without further purification. ¹H NMR (400 MHz, DMSO-d₆) δ 7.56 (br. s, 1H), 4.44 (td, J 8.2, 1.0 Hz, 1H), 4.06-3.97 (m, 1H), 3.95-3.88 (m, 1H), 1.93-1.80 (m, 2H), 1.35 (s, 3H), 1.30 (s, 3H). ESI-MS m/z calc. 161.0852, found 162.2 (M+1)⁺; Retention time: 1.28 minutes; LC method X.

### Step 5: (2R)-2-Amino-4-fluoro-4-methyl-pentan-1-ol

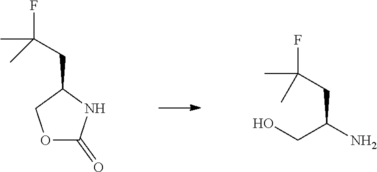

To a solution of potassium hydroxide (2.5 g, 44.559 mmol) in ethanol (15 mL) and water (1.5 mL) was added (4R)-4-(2-fluoro-2-methyl-propyl)oxazolidin-2-one (2.3 g, 13.557 mmol). The reaction mixture was heated at 100° C. for 4 h. The reaction was then allowed to reach room temperature and concentrated under reduced pressure. The residue was co-evaporated with toluene (3×10 mL) to afford a pale orange residue which was filtered on a celite pad, washed with dichloromethane (3×20 mL). The filtrate was concentrated under reduced pressure to afford (2R)-2-amino-4-fluoro-4-methyl-pentan-1-ol (1.82 g, 94%) as a dark orange oil which was directly used in the next step without further purification ¹H NMR (400 MHz, CD₃OD) δ 3.50 (dd, J 10.6, 4.8 Hz, 1H), 3.35-3.27 (m, 1H), 3.17-3.09 (m, 1H), 1.77-1.59 (m, 2H), 1.42 (s, 3H), 1.37 (s, 3H).19F NMR (377 MHz, CD₃OD) δ −139.34 (s, 1F). ESI-MS m/z calc. 135.10594, found 136.2 (M+1)⁺; Retention time: 0.23 minutes; LC method X.

### Step 6: 3-[[4-[(2R)-2-Amino-4-fluoro-4-methyl-pentoxy]-6-(2,6-dimethylphenyl)pyrimidin-2-yl]sulfamoyl]benzoic acid

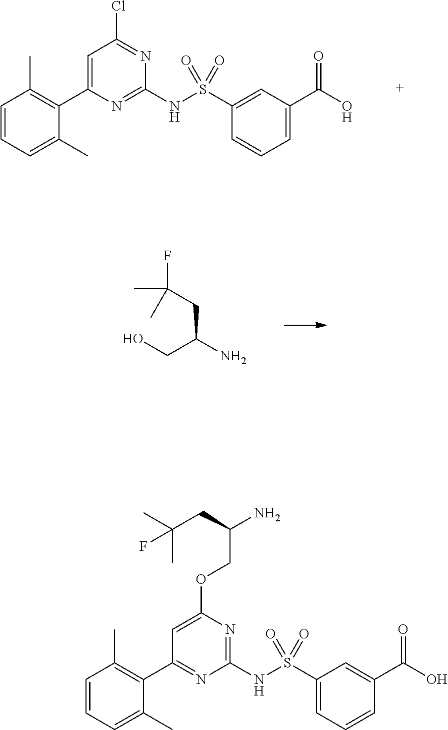

3-[[4-Chloro-6-(2,6-dimethylphenyl)pyrimidin-2-yl]sulfamoyl]benzoic acid (1 g, 2.393 mmol) and (2R)-2-amino-4-fluoro-4-methyl-pentan-1-ol (391 mg, 2.892 mmol) were combined under nitrogen in anhydrous THF (9 mL). To the resulting cloudy solution sodium tert-butoxide (1.05 g, 10.93 mmol) was added in one portion resulting in the dissolution of the solids and a slightly exothermic reaction. The mixture was stirred at room temperature for 2.5 h. The reaction was diluted with ethyl acetate (20 mL), HCl (20 mL of 1 M, 20.00 mmol) and brine (20 mL) and the resulting two phases were separated. The aqueous phase was further extracted with EtOAc (3×15 mL). The combined organic extracts were dried over sodium sulfate and concentrated. The residue was triturated in a mixture of EtOAc and hexanes (1:3, v:v) and the resulting suspension was stirred at rt overnight. The solid was filtered and dried to give 3-[[4-[(2R)-2-amino-4-fluoro-4-methyl-pentoxy]-6-(2,6-dimethylphenyl)pyrimidin-2-yl]sulfamoyl]benzoic acid (hydrochloride salt) (1.238 g, 94%) as a tan solid. ESI-MS m/z calc. 516.18427, found 517.45 (M+1)⁺; Retention time: 0.99 minutes; LC method A.

### Step 7: 3-[[4-(2,6-Dimethylphenyl)-6-[(2R)-4-fluoro-2-[(5-isopropoxypyrimidin-2-yl)methylamino]-4-methyl-pentoxy]pyrimidin-2-yl]sulfamoyl]benzoic acid

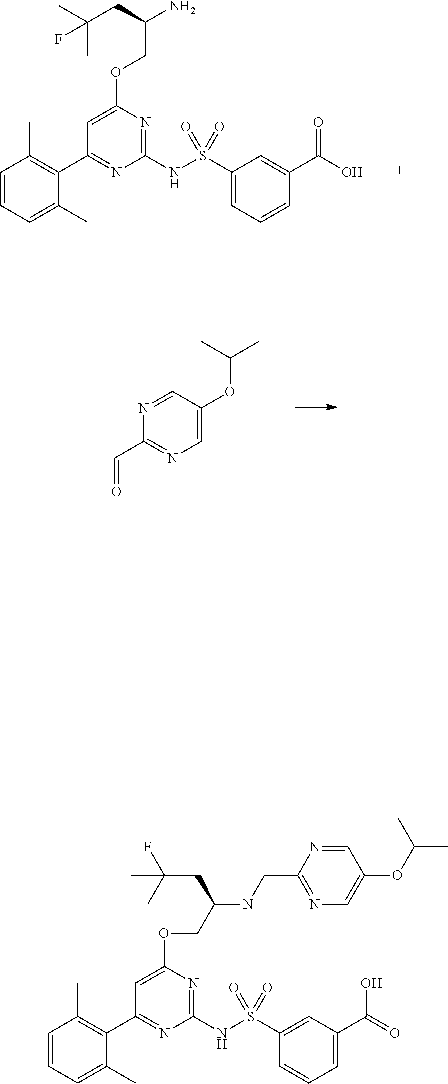 [SMILES: Cc1cccc(C)c1-c1cc(OC[C@H](N)CC(C)(C)F)nc(NS(=O)(=O)c2cccc(C(=O)O)c2)n1 ; CC(C)Oc1cnc(C=O)nc1 ; Cc1cccc(C)c1-c1cc(OC[C@@H](CC(C)(C)F)NCc2ncc(OC(C)C)cn2)nc(NS(=O)(=O)c2cccc(C(=O)O)c2)n1]

A stirred solution of 3-[[4-[(2R)-2-amino-4-fluoro-4-methyl-pentoxy]-6-(2,6-dimethylphenyl)pyrimidin-2-yl]sulfamoyl]benzoic acid (hydrochloride salt) (100 mg, 0.1806 mmol) in anhydrous dichloromethane (5 mL) was stirred at 0° C. for 15 minutes after which time 5-isopropoxypyrimidine-2-carbaldehyde (29 mg, 0.1745 mmol) was added. The mixture was stirred at 0° C. for 15 minutes. Then sodium triacetoxyborohydride (115 mg, 0.5426 mmol) was added and the reaction was stirred at 0° C. for 30 minutes. Then the reaction was quenched by the addition of a saturated aqueous solution of 1N hydrochloric acid (5 mL) and concentrated under reduced pressure. The crude was purified by reverse phase chromatography on a C₁₈ using a 30 g HP gold column and eluting with a gradient of acetonitrile in acidic water (containing 0.1% v/v of hydrochloric acid, 5 to 100% in 15 CV). The desired fractions were concentrated under reduced pressure. The residual water was co-evaporated with methanol, then freeze-dried to afford 3-[[4-(2,6-dimethylphenyl)-6-[(2R)-4-fluoro-2-[(5-isopropoxypyrimidin-2-yl)methylamino]-4-methyl-pentoxy]pyrimidin-2-yl]sulfamoyl]benzoic acid (hydrochloride salt) (55 mg, 39%) as a white powder. ESI-MS m/z calc. 666.2636, found 667.3 (M+1)⁺; Retention time: 2.48 minutes; LC method 1D.

### Step 8: (11R)-6-(2,6-Dimethylphenyl)-11-(2-fluoro-2-methyl-propyl)-12-[(5-isopropoxypyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1308)

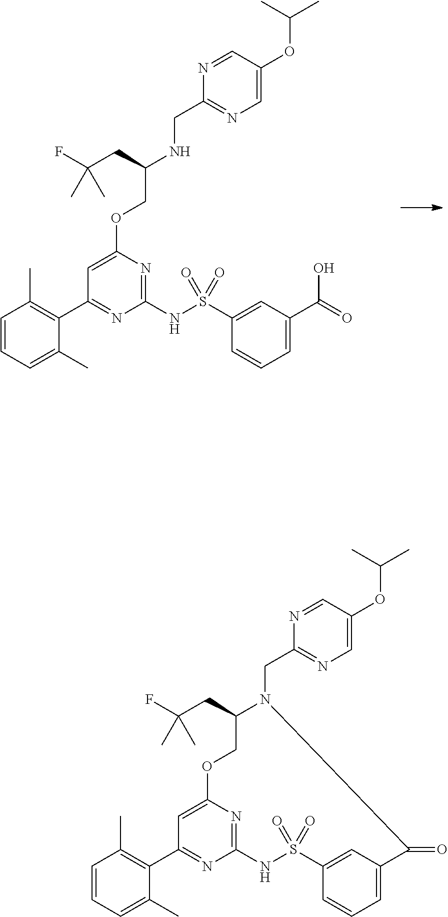 [SMILES: Cc1cccc(C)c1-c1cc(OC[C@@H](CC(C)(C)F)NCc2ncc(OC(C)C)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ncc(OC(C)C)cn1)[C@H](CC(C)(C)F)CO2]

A stirred solution of 3-[[4-(2,6-dimethylphenyl)-6-[(2R)-4-fluoro-2-[(5-isopropoxypyrimidin-2-yl)methylamino]-4-methyl-pentoxy]pyrimidin-2-yl]sulfamoyl]benzoic acid (hydrochloride salt) (55 mg, 0.0782 mmol) in anhydrous N,N-dimethylformamide (2 mL) under nitrogen atmosphere was cooled down to 0° C. Diisopropylethylamine (74.200 mg, 100 L, 0.5741 mmol) was slowly added, followed by 2-chloro-1-methylpyridinium iodide (40 mg, 0.1566 mmol). The reaction was stirred at 0° C. for 15 minutes then allowed to reach room temperature and stirred overnight at room temperature. The reaction was then concentrated under reduced pressure and purified by reverse phase chromatography on a C₁₈ using a 30 g HP Gold column and eluting with a gradient of acetonitrile in acidic water (containing 0.1% v/v of formic acid, 40 to 100% in 18CV). The desired fractions were concentrated under reduced pressure then freeze-dried to afford a pink fluffy powder which was purified by reverse phase chromatography on a C₁₈ using a 30 g HP Gold column and eluting with a gradient of acetonitrile in acidic water (containing 0.1% v/v of formic acid, 40 to 80% in 25CV). The desired fractions were concentrated under reduced pressure, the freeze-dried to afford (11R)-6-(2,6-dimethylphenyl)-11-(2-fluoro-2-methyl-propyl)-12-[(5-isopropoxypyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (17 mg, 31%) as an off-white powder. ESI-MS m/z calc. 648.253, found 649.3 (M+1)⁺; Retention time: 4.04 minutes; LC method Y.

The product (11R)-6-(2,6-dimethylphenyl)-11-(2-fluoro-2-methyl-propyl)-12-[(5-isopropoxypyrimidin-2-yl)methyl]-2,2-dioxo-9-oxa-2⁶-thia-3,5,12,19-tetrazatricyclo[12.3.1.14,8]nonadeca-1(18),4(19),5,7,14,16-hexaen-13-one (17 mg, 0.0245 mmol) was further purified by chiral SFC (Flow rate: 75 mL/min, 50% MeOH, Column Lux 5 m, i-Amylose 3, 250×21.2 mm, temperature=40° C., outlet pressure: 100 bar, injection volume: 1000 μL, 8.5 mg/injection, concentration 7 mg/mL). The pure fractions were concentrated in vacuo and the product was lyophilized to afford (11R)-6-(2,6-dimethylphenyl)-11-(2-fluoro-2-methyl-propyl)-12-[(5-isopropoxypyrimidin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (11 mg, 69%) as a white solid. ¹H NMR (400 MHz, DMSO-d₆) δ 13.07 (br. s., 1H), 8.66 (br. s., 1H), 8.52 (s, 2H), 7.98-7.87 (m, 1H), 7.74-7.50 (m, 2H), 7.24 (br. s., 1H), 7.18-7.05 (m, 2H), 6.33 (br. s., 1H), 5.43-5.31 (m, 1H), 4.88 (d, J 16.6 Hz, 1H), 4.81 (dt, J 12.1, 5.9 Hz, 1H), 4.63 (d, J 16.6 Hz, 1H), 4.23 (br. s., 2H), 2.29-1.85 (m, 7H), 1.82-1.69 (m, 1H), 1.30 (dd, J 6.0, 2.1 Hz, 6H), 1.16-1.06 (m, 3H), 0.93-0.82 (m, 3H). ESI-MS m/z calc. 648.253, found 649.3 (M+1)⁺; Retention time: 4.04 minutes; LC method Y.

### Example 151: Preparation of (Compound 1309)

### Step 1: (2R)-2-Amino-5-methyl-hexan-1-ol

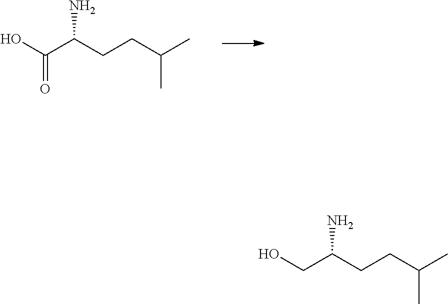

Borane tetrahydrofuran complex in THF (58 mL of 1 M, 58.000 mmol) was slowly added to a suspension of (2R)-2-amino-5-methyl-hexanoic acid (4.05 g, 27.893 mmol) in 2-methyltetrahydrofuran (40 mL). The reaction was stirred at room temperature for 16 h. Aqueous HCl (28 mL of 3 M, 84.00 mmol) was added keeping the temperature under 25° C. and the reaction was stirred at room temperature for 45 minutes. MeTHF (100 mL) was added and the excess THF was removed by evaporation. The solution was basified, at pH around 9, with NaOH 25% aqueous solution (10 mL). The organic phase was separated. The aqueous layer was extracted with MeTHF (2×50 mL). The combined organic layers were washed with brine (50 mL), dried over anhydrous sodium sulfate, filtered, and concentrated under vacuum. Aqueous HCl (14 mL of 3 M, 42.000 mmol) was added to the residue, water was evaporated to dryness and then co-evaporated with isopropanol (3×50 mL). MTBE (100 mL) was added to the residue and the solvent was evaporated to dryness to afford (2R)-2-amino-5-methyl-hexan-1-ol (hydrochloride salt) (2.231 g, 48%) as a white solid. ¹H NMR (400 MHz, DMSO-d₆) δ 7.82 (br. s., 3H), 5.26 (t, J 4.9 Hz, 1H), 3.58 (dt, J 11.2, 4.4 Hz, 1H), 3.42 (dt, J 11.3, 5.7 Hz, 1H), 3.00 (br. s., 1H), 1.57-1.43 (m, 3H), 1.25-1.15 (m, 2H), 0.86 (d, J 6.1 Hz, 6H). ESI-MS m/z calc. 131.131, found 132.2 (M+1)⁺; Retention time: 2.022 minutes. To the aqueous phase, NaOH 25% aqueous (3 mL) was added and the aqueous layer was extracted with MeTHF (2×50 mL). The combined organic layers were washed with brine (50 mL), dried over anhydrous sodium sulfate, filtered, and concentrated under vacuum. Aqueous HCl (14 mL of 3 M, 42.000 mmol) was added to the residue, water was evaporated to dryness and then co-evaporated with isopropanol (3×50 mL). MTBE (100 mL) was added to the residue and the solvent was evaporated to dryness to afford a second batch of (2R)-2-amino-5-methyl-hexan-1-ol (hydrochloride salt) (2.227 g, 45%) as a white solid. ¹H NMR (400 MHz, DMSO-d₆) δ 7.94 (br. s., 3H), 5.27 (t, J 5.0 Hz, 1H), 3.58 (dt, J 11.4, 4.3 Hz, 1H), 3.43 (dt, J 11.5, 5.7 Hz, 1H), 3.06-2.93 (m, 1H), 1.58-1.43 (m, 3H), 1.25-1.15 (m, 2H), 0.86 (d, J 6.4 Hz, 6H). ESI-MS m/z calc. 131.131, found 132.2 (M+1)⁺; Retention time: 1.994 minutes. Total quantity of 4.458 g and total yield of 95%. LC method 1D.

### Step 2: 3-[[4-[(2R)-2-Amino-5-methyl-hexoxy]-6-(2,6-dimethylphenyl)pyrimidin-2-yl]sulfamoyl]benzoic acid

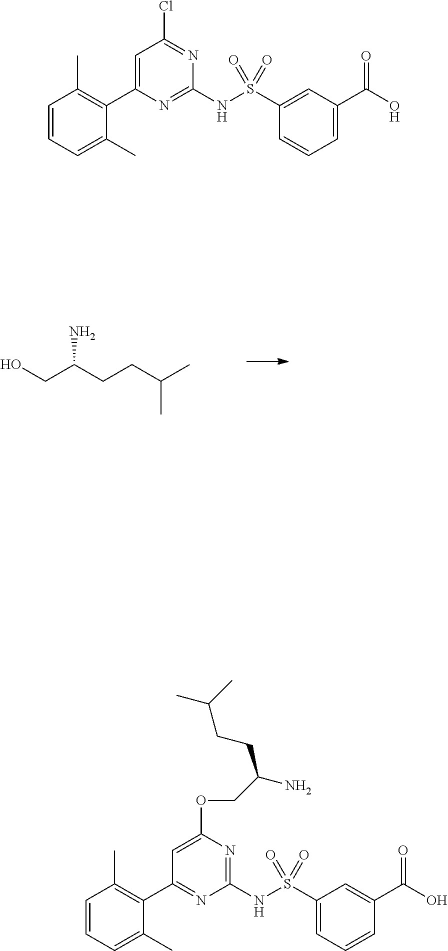

3-[[4-Chloro-6-(2,6-dimethylphenyl)pyrimidin-2-yl]sulfamoyl]benzoic acid (1.1 g, 2.632 mmol) and (2R)-2-amino-5-methyl-hexan-1-ol (hydrochloride salt) (538 mg, 3.209 mmol) were combined under nitrogen in anhydrous THF (10 mL). To the resulting cloudy solution sodium tert-butoxide (1.01 g, 10.51 mmol) was added in one portion resulting in fast dissolution of the solids and a slightly exothermic reaction. The mixture was stirred at room temperature for 1 h. More (2R)-2-amino-5-methyl-hexan-1-ol (hydrochloride salt) (86 mg, 0.5129 mmol) and sodium tert-butoxide (Sodium salt) (140 mg, 1.457 mmol) were added and the mixture was stirred at rt for 1.5 h. The reaction was diluted with ethyl acetate (20 mL), HCl (20 mL of 1 M, 20.00 mmol) and brine (20 mL) and the resulting two phases were separated. The aqueous phase was further extracted with EtOAc (3×15 mL). The combined organic extracts were dried over sodium sulfate and concentrated. The residue was triturated in a mixture of EtOAc and hexanes (1:3, v:v) and the resulting suspension was stirred at rt for 1 h. The solid was filtered and dried to give 3-[[4-[(2R)-2-amino-5-methyl-hexoxy]-6-(2,6-dimethylphenyl)pyrimidin-2-yl]sulfamoyl]benzoic acid (hydrochloride salt) (1.32 g, 82%) as an off-white solid. ESI-MS m/z calc. 512.20935, found 513.59 (M+1)⁺; Retention time: 1.1 minutes; LC method A.

### Step 3: (11R)-12-[[5-(3,3-Dimethylazetidin-1-yl)pyrimidin-2-yl]methyl]-6-(2,6-dimethylphenyl)-11-isopentyl-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1309)

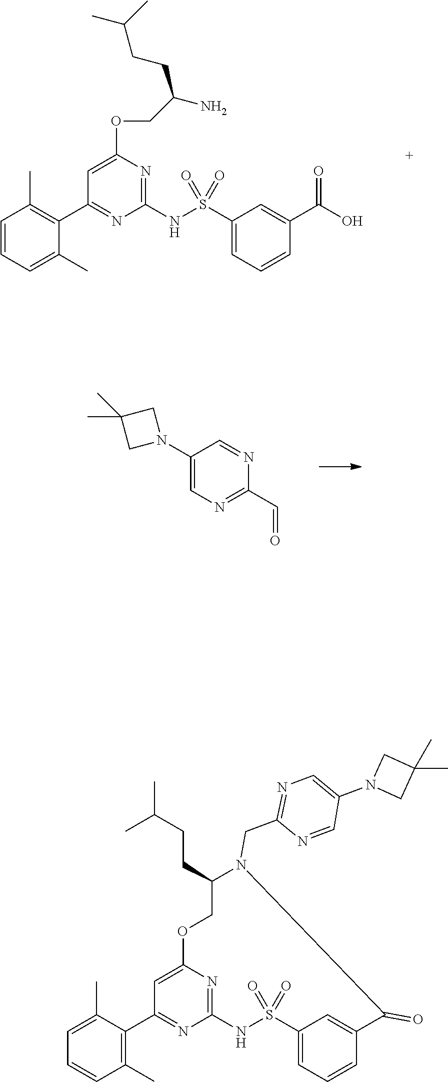 [SMILES: Cc1cccc(C)c1-c1cc(OC[C@H](N)CCC(C)C)nc(NS(=O)(=O)c2cccc(C(=O)O)c2)n1 ; CC1(C)CN(c2cnc(C=O)nc2)C1 ; Cc1cccc(C)c1-c1cc2nc(n1)NS(=O)(=O)c1cccc(c1)C(=O)N(Cc1ncc(N3CC(C)(C)C3)cn1)[C@H](CCC(C)C)CO2]

3-[[4-[(2R)-2-Amino-5-methyl-hexoxy]-6-(2,6-dimethylphenyl)pyrimidin-2-yl]sulfamoyl]benzoic acid (hydrochloride salt) (170 mg, 0.2786 mmol), 5-(3,3-dimethylazetidin-1-yl)pyrimidine-2-carbaldehyde (64.4 mg, 0.3166 mmol), and acetic acid (15.5 μL, 0.2726 mmol) were combined in dichloromethane (1.2 mL). The mixture was cooled down in an ice bath. Diisopropylethyl amine (100 μL, 0.5741 mmol) was added (partial dissolution of solids) followed quickly by sodium triacetoxyborohydride (189.6 mg, 0.8946 mmol). The reaction was vigorously stirred at 0° C. for 30 min. The reaction was then quenched at 0° C. with 1N HCl, then partitioned with ethyl acetate. The aqueous phase was extracted with ethyl acetate (3×), then the combined organic phases were dried over anhydrous sodium sulfate, filtered, and evaporated to dryness. The residue was purified by flash chromatography on silica gel (24 g 0-15% methanol in dichloromethane). After evaporation, the residue was azeotroped with dichloromethane/hexanes to remove any residual methanol and provide 3-[[4-[(2R)-2-[[5-(3,3-dimethylazetidin-1-yl)pyrimidin-2-yl]methylamino]-5-methyl-hexoxy]-6-(2,6-dimethylphenyl)pyrimidin-2-yl]sulfamoyl]benzoic acid (hydrochloride salt) (140.7 mg, 69%) as a white solid. ESI-MS m/z calc. 687.3203, found 688.5 (M+1)⁺; Retention time: 1.45 minutes; LC method A. 3-[[4-[(2R)-2-[[5-(3,3-Dimethylazetidin-1-yl)pyrimidin-2-yl]methylamino]-5-methyl-hexoxy]-6-(2,6-dimethylphenyl)pyrimidin-2-yl]sulfamoyl]benzoic acid (hydrochloride salt) (140.7 mg, 69%) and 2-chloro-4,6-dimethoxy-1,3,5-triazine (42.1 mg, 0.2398 mmol) were combined in anhydrous DMF (10 mL) and cooled to 0° C. 4-methylmorpholine (65 μL, 0.5912 mmol) was added and the reaction allowed to slowly warm to room temperature overnight. The reaction was concentrated under reduced pressure, filtered, and purified by UV-triggered reverse-phase HPLC eluting with a 1-99% acetonitrile/water gradient over 20 minutes with 5 mM HCl acid modifier in the aqueous phase to give pure fractions which were then concentrated to remove the acetonitrile. The aqueous phase was extracted with dichloromethane (3×) and the organic phase dried over anhydrous magnesium sulfate, filtered, and concentrated in vacuo to give (11R)-12-[[5-(3,3-dimethylazetidin-1-yl)pyrimidin-2-yl]methyl]-6-(2,6-dimethylphenyl)-11-isopentyl-2,2-dioxo-9-oxa-2?⁶-thia-3,5,12,19-tetrazatricyclo[12.3.1.14,8]nonadeca-1(18),4(19),5,7,14,16-hexaen-13-one (69.2 mg, 37%) as a white solid. ¹H NMR (400 MHz, DMSO) δ 12.47 (d, J 335.8 Hz, 1H), 8.60 (s, 1H), 8.03 (s, 2H), 7.89 (s, 1H), 7.63 (s, 2H), 7.24 (s, 1H), 7.11 (s, 2H), 6.35 (s, 1H), 5.32 (s, 1H), 4.90 (d, J 16.3 Hz, 1H), 4.46 (d, J 16.2 Hz, 1H), 4.07 (s, 1H), 3.93 (s, 1H), 3.64 (s, 4H), 2.01 (s, 4H), 1.73 (s, 1H), 1.51 (s, 1H), 1.29 (s, 6H), 1.16-0.96 (m, 3H), 0.90-0.78 (m, 2H), 0.71 (d, J 6.4 Hz, 3H), 0.64 (d, J 6.4 Hz, 3H). ESI-MS m/z calc. 687.3203, found 688.5 (M+1)⁺; Retention time: 1.45 minutes; LC method A.

### Example 152: Preparation of Compound 1310

### Step 1: 3-[[4-[(2R)-2-[[6-[Cyclobutyl(methyl)amino]pyrazin-2-yl]methylamino]-4,4-dimethyl-pentoxy]-6-(2,6-dimethylphenyl)pyrimidin-2-yl]sulfamoyl]benzoic acid

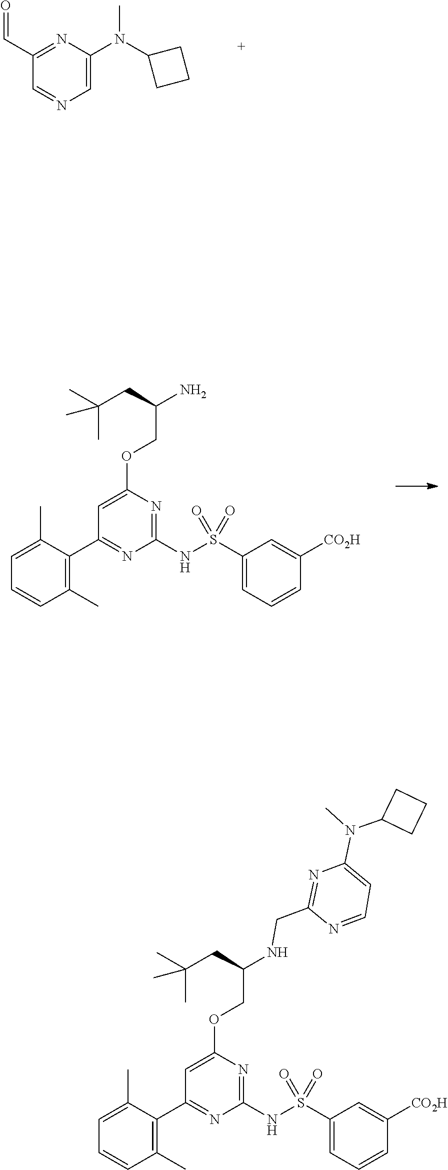 [SMILES: CN(c1cncc(C=O)n1)C1CCC1 ; Cc1cccc(C)c1-c1cc(OC[C@H](N)CC(C)(C)C)nc(NS(=O)(=O)c2cccc(C(=O)O)c2)n1 ; Cc1cccc(C)c1-c1cc(OC[C@@H](CC(C)(C)C)NCc2nccc(N(C)C3CCC3)n2)nc(NS(=O)(=O)c2cccc(C(=O)O)c2)n1]

To a stirring suspension of 3-[[4-[(2R)-2-amino-4,4-dimethyl-pentoxy]-6-(2,6-dimethylphenyl)pyrimidin-2-yl]sulfamoyl]benzoic acid (hydrochloride salt) (228 mg, 0.4152 mmol) and 6-[cyclobutyl(methyl)amino]pyrazine-2-carbaldehyde (114 mg, 0.5306 mmol) in DCM (4.5 mL) at 0° C. was added sodium triacetoxyborohydride (223 mg, 1.0206 mmol) in one portion. The reaction was stirred from 0° C. to rt for 4 h. The reaction was cooled down to 0° C., 1 N aqueous solution of HCl was added (50 mL) and the mixture was stirred 20 min. The two phases were separated, and the aqueous phase was extracted with EtOAc (3×10 mL). The organic phases were combined, dried over sodium sulfate, filtered and evaporated under reduced pressure. The residue was subsequently purified by reverse flash chromatography on a 30 g Cis Gold cartridge using a gradient of MeCN (15% for 3.5 CV then to 100% over 20 CV then 100% for 3 CV) in acidic water (HCl=0.1% w/w) to provide the pure 3-[[4-[(2R)-2-[[6-[cyclobutyl(methyl)amino]pyrazin-2-yl]methylamino]-4,4-dimethyl-pentoxy]-6-(2,6-dimethylphenyl)pyrimidin-2-yl]sulfamoyl]benzoic acid (hydrochloride salt) (188 mg, 54%) as a yellow gum. ESI-MS m/z calc. 687.3203, found 688.2 (M+1)⁺; Retention time: 1.49 minutes; LC method X.

### Step 2: (11R)-12-[[6-[Cyclobutyl(methyl)amino]pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1310)

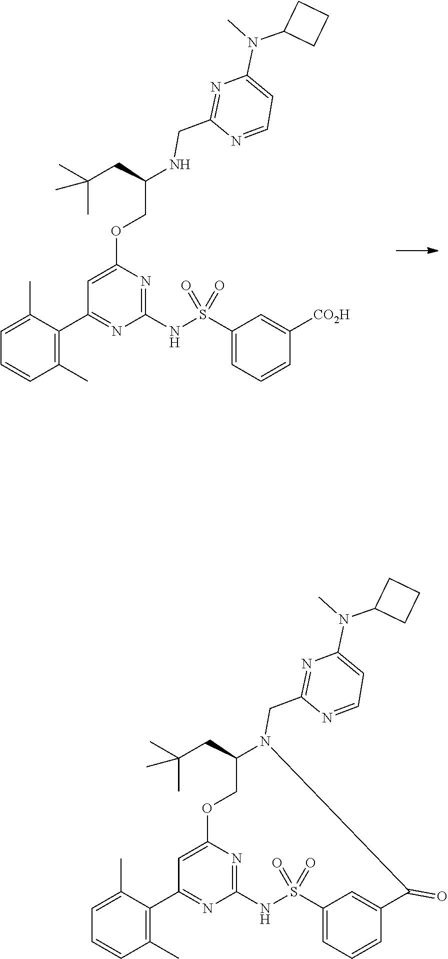 [SMILES: Cc1cccc(C)c1-c1cc(OC[C@@H](CC(C)(C)C)NCc2nccc(N(C)C3CCC3)n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1nccc(N(C)C3CCC3)n1)[C@H](CC(C)(C)C)CO2]

To a stirred solution of N-methylmorpholine (174.80 mg, 0.19 mL, 1.7282 mmol) in dimethylformamide (DMF) (40 mL) at 0° C. was added 2-chloro-4,6-dimethoxy-1,3,5-triazine (97 mg, 0.5525 mmol) followed by 3-[[4-[(2R)-2-[[6-[cyclobutyl(methyl)amino]pyrazin-2-yl]methylamino]-4,4-dimethyl-pentoxy]-6-(2,6-dimethylphenyl)pyrimidin-2-yl]sulfamoyl]benzoic acid (hydrochloride salt) (188 mg, 0.2258 mmol) in dimethylformamide (DMF) (4 mL). The reaction mixture was stirred at 0° C. for 5 minutes. Then the reaction was warmed up to room temperature and stirred at room temperature for 24 hours. After 24 hours the volatiles were removed under reduced pressure and the residue was subsequently purified by reverse flash chromatography on a 30 g C₁₈ Gold cartridge using a gradient of MeCN (10% for 3.5 CV then to 100% over 20 CV then 100% for 3.5 CV) in acidic water (formic acid=0.1% w/w) to provide the pure (11R)-12-[[6-[cyclobutyl(methyl)amino]pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (111.61 mg, 72%) as a white solid. ¹H NMR (400 MHz, DMSO-d₆) δ 12.91 (br. s, 1H), 8.68 (s, 1H), 8.03-7.82 (m, 3H), 7.68 (br. s, 2H), 7.32-7.20 (m, 1H), 7.12 (d, J=7.3 Hz, 2H), 6.44 (br. s, 1H), 5.40 (dd, J 10.8, 4.2 Hz, 1H), 4.89-4.66 (m, 2H), 4.45 (d, J 15.7 Hz, 1H), 4.31 (t, J 11.0 Hz, 1H), 4.05 (br. s., 1H), 3.06 (s, 3H), 2.24-1.84 (m, 10H), 1.76 (dd, J 15.4, 9.0 Hz, 1H), 1.70-1.58 (m, 2H), 1.42 (d, J 14.9 Hz, 1H), 0.56 (s, 9H). ESI-MS m/z calc. 669.30975, found 670.2 (M+1)⁺; Retention time: 4.64 minutes; LC method Y.

### Example 153: Preparation of Compound 1311

### Step 1: Methyl (2R)-2-(benzyloxycarbonylamino)hex-5-enoate

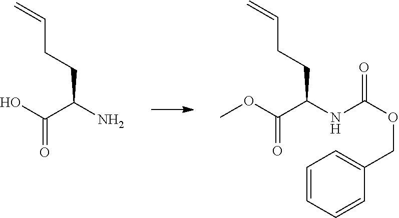

(2R)-2-Aminohex-5-enoic acid (2 g, 15.485 mmol) was mixed in MeOH (40 mL) and cooled in a ˜−10° C. acetone dry ice bath. thionyl chloride (4.0775 g, 2.5 mL, 34.273 mmol) was added dropwise. The clear mixture was then let reach rt and stirred for 24 h. It was then concentrated. The off-white solid obtained was taken into DCM (30 mL) and Water (15 mL) and cooled in ice water bath. Sodium bicarbonate (8.6 g, 102.37 mmol) was added, followed by CbzOSu (4.68 g, 18.779 mmol). The yellowish mixture was stirred efficiently for 15 h (ice bath temperature reaching rt during the course). DCM and water (50 ml each) were added. Layers were separated. The DCM solution was dried over anhydrous magnesium sulfate, filtered and concentrated. The residue was purified by silica gel chromatography (80 g column), using 5-40% EtOAc in Hexanes, to afford methyl (2R)-2-(benzyloxycarbonylamino)hex-5-enoate (4.23 g, 94%) as colorless oil. ESI-MS m/z calc. 277.1314, found 278.3 (M+1)⁺; Retention time: 2.86 minutes; LC method T.

### Step 2: Methyl (2R)-2-(benzyloxycarbonylamino)-4-cyclopropyl-butanoate

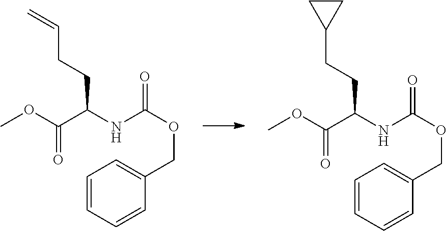 [SMILES: C=CCC[C@@H](NC(=O)OCc1ccccc1)C(=O)OC ; COC(=O)[C@@H](CCC1CC1)NC(=O)OCc1ccccc1]

Et₂Zn in hexanes (50 mL of 1 M, 50.000 mmol) was diluted with DCM (25 mL) and cooled to ˜−10° C. TFA (5.7720 g, 3.9 mL, 50.621 mmol) in DCM (10 mL) was added dropwise. The mixture was stirred at <0° C. for 15 min. CH₂I₂ (12.968 g, 3.9 mL, 48.418 mmol) in DCM (25 mL) was added in portions. The mixture was stirred at the same temperature for 15 min. Methyl (2R)-2-(benzyloxycarbonylamino)hex-5-enoate (3.9 g, 13.360 mmol) in DCM (25 mL) was then added in portions. The mixture was stirred for 15 h (reaching rt gradually). HCl (0.2 N aqueous) was added in portions (40 ml total). More DCM (60 ml) was added. Layers were separated. The organic layer was washed with brine, dried over anhydrous magnesium sulfate, filtered and concentrated. The residue oil was purified by silica gel chromatography (80 g column), using 0-40% EtOAc in Hexanes, to afford methyl (2R)-2-(benzyloxycarbonylamino)-4-cyclopropyl-butanoate (3.65 g, 89%) as colorless oil. ¹H NMR (500 MHz, Chloroform-d) δ 7.43-7.28 (m, 5H), 5.31-5.20 (m, 1H), 5.11 (s, 2H), 4.49-4.35 (m, 1H), 3.74 (s, 3H), 2.02-1.86 (m, 1H), 1.83-1.67 (m, 1H), 1.32-1.19 (m, 2H), 0.77-0.59 (m, 1H), 0.52-0.35 (m, 2H), 0.09-−0.04 (m, 2H). ESI-MS m/z calc. 291.14706, found 292.5 (M+1)⁺; Retention time: 3.01 minutes; LC method T.

### Step 3: Benzyl N-[(1R)-3-cyclopropyl-1-(hydroxymethyl)propyl]carbamate

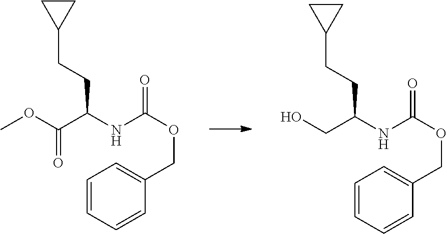 [SMILES: COC(=O)[C@@H](CCC1CC1)NC(=O)OCc1ccccc1 ; O=C(N[C@@H](CO)CCC1CC1)OCc1ccccc1]

Methyl (2R)-2-(benzyloxycarbonylamino)-4-cyclopropyl-butanoate (3.94 g, 12.847 mmol) was dissolved in THF (40 mL) and the solution was cooled in ice water bath and stirred under a nitrogen balloon. LiBH₄ in THF (12 mL of 2 M, 24.000 mmol) was added in small portions over 10 min. The ice bath was removed and the mixture was stirred at rt for 2 h. NH₄Cl (20 ml, saturated aqueous) was added, followed by EtOAc (50 ml) and water (40 ml). Layers were separated. The organic layer was washed with more water (30 ml×2), brine, dried over anhydrous magnesium sulfate, filtered and concentrated to afford crude benzyl N-[(1R)-3-cyclopropyl-1-(hydroxymethyl)propyl]carbamate (3.75 g, 100%) as colorless oil. ESI-MS m/z calc. 263.15213, found 264.4 (M+1)⁺; Retention time: 2.65 minutes; LC method T.

### Step 4: (2R)-2-Amino-4-cyclopropyl-butan-1-ol

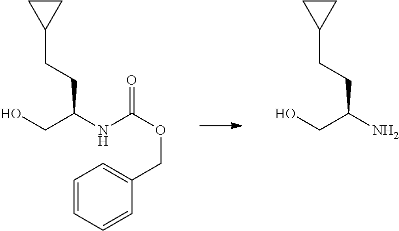 [SMILES: O=C(N[C@@H](CO)CCC1CC1)OCc1ccccc1 ; N[C@@H](CO)CCC1CC1]

Benzyl N-[(1R)-3-cyclopropyl-1-(hydroxymethyl)propyl]carbamate (3.75 g, 12.817 mmol) was dissolved in EtOH (60 mL). HCl aqueous (12.9 mL of 1 M, 12.900 mmol) was added, followed by Pd on activated carbon (300 mg, 5% w/w, 0.1410 mmol). The mixture was vacuumed and refilled with H2 balloon and stirred at rt for 4 h. It was then filtered through a celite pad and washed with MeOH. The combined filtrate was concentrated to give (2R)-2-amino-4-cyclopropyl-butan-1-ol (hydrochloride salt) (2.15 g, 96%) as a colorless oil. ESI-MS m/z calc. 129.11537, found 130.4 (M+1)⁺; Retention time: 1.09 minutes; LC method T.

### Step 5: 3-[[4-[(2R)-2-Amino-4-cyclopropyl-butoxy]-6-(2,6-dimethylphenyl)pyrimidin-2-yl]sulfamoyl]benzoic acid

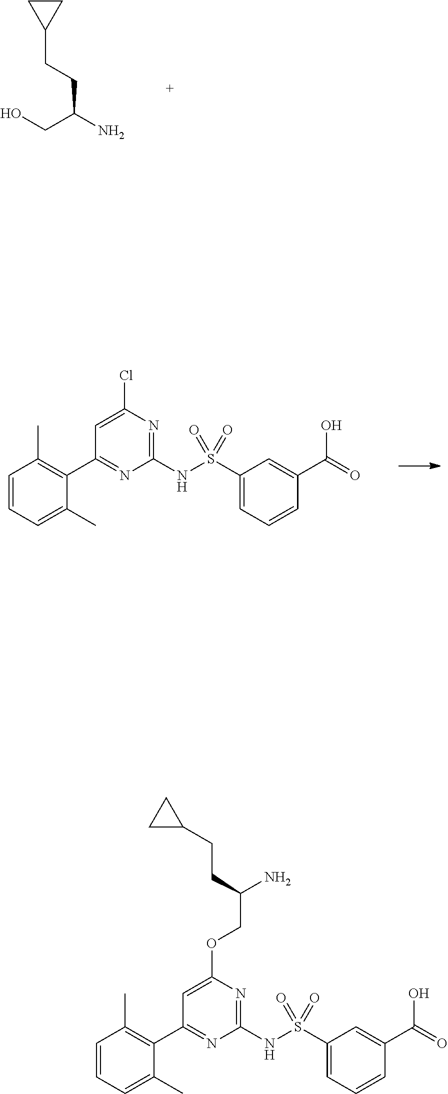 [SMILES: N[C@@H](CO)CCC1CC1 ; Cc1cccc(C)c1-c1cc(Cl)nc(NS(=O)(=O)c2cccc(C(=O)O)c2)n1 ; Cc1cccc(C)c1-c1cc(OC[C@H](N)CCC2CC2)nc(NS(=O)(=O)c2cccc(C(=O)O)c2)n1]

3-[[4-chloro-6-(2,6-Dimethylphenyl)pyrimidin-2-yl]sulfamoyl]benzoic acid (3 g, 7.1793 mmol) and (2R)-2-amino-4-cyclopropyl-butan-1-ol (hydrochloride salt) (2.15 g, 12.329 mmol) were mixed in THF (20 mL) at rt. Sodium t-butoxide (2.8 g, 29.135 mmol) was added in one portion. The mixture was stirred at rt for 1 h. More sodium t-butoxide (1.4 g, 14.568 mmol) was added. The mixture was stirred at rt for 2 h. HCl aqueous (60 mL of 1 M, 60.000 mmol) was added, followed by EtOAc (60 ml). Layers were separated. The aqueous layer was extracted with more EtOAc (20 ml). The combined EtOAc solution was washed with brine, dried over anhydrous magnesium sulfate, filtered and concentrated. The residue was retaken into EtOAc (˜20 ml) and sonicated briefly. The supernatant was discarded. The precipitate was dissolved in THF and transferred into shipment vial and dried under high vacuum for 20 h to afford 3-[[4-[(2R)-2-amino-4-cyclopropyl-butoxy]-6-(2,6-dimethylphenyl)pyrimidin-2-yl]sulfamoyl]benzoic acid (hydrochloride salt) (4.14 g, 95%) as slightly yellowish solid. ¹H NMR (500 MHz, DMSO-d₆) δ 13.20 (s, 1H), 8.44 (s, 1H), 8.28-8.05 (m, 5H), 7.69 (t, J 0.8, 7.8 Hz, 1H), 7.25 (t, J 7.6, 7.6 Hz, 1H), 7.12 (d, J 7.6 Hz, 2H), 6.30 (s, 1H), 4.36 (dd, J 11.8, 3.3 Hz, 1H), 4.21 (dd, J 11.8, 6.6 Hz, 1H), 3.57-3.48 (m, 1H), 1.99 (d, J 8.5 Hz, 6H), 1.72-1.63 (m, 2H), 1.33-1.19 (m, 2H), 0.77-0.61 (m, 1H), 0.47-0.34 (m, 2H), 0.08-−0.01 (m, 2H). ESI-MS m/z calc. 510.1937, found 511.8 (M+1)⁺; Retention time: 1.78 minutes; LC method W.

### Step 6: 3-[[4-[(2R)-4-Cyclopropyl-2-[[5-(4,4-dimethyl-1-piperidyl)pyrimidin-2-yl]methylamino]butoxy]-6-(2,6-dimethylphenyl)pyrimidin-2-yl]sulfamoyl]benzoic acid

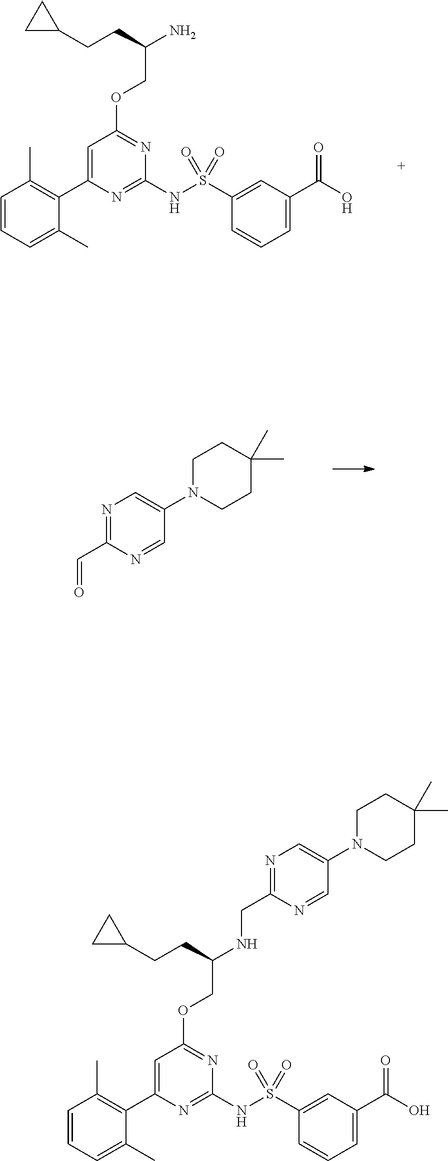 [SMILES: Cc1cccc(C)c1-c1cc(OC[C@H](N)CCC2CC2)nc(NS(=O)(=O)c2cccc(C(=O)O)c2)n1 ; CC1(C)CCN(c2cnc(C=O)nc2)CC1 ; Cc1cccc(C)c1-c1cc(OC[C@@H](CCC2CC2)NCc2ncc(N3CCC(C)(C)CC3)cn2)nc(NS(=O)(=O)c2cccc(C(=O)O)c2)n1]

3-[[4-[(2R)-2-Amino-4-cyclopropyl-butoxy]-6-(2,6-dimethylphenyl)pyrimidin-2-yl]sulfamoyl]benzoic acid (hydrochloride salt) (125 mg, 0.2285 mmol) and 5-(4,4-dimethyl-1-piperidyl)pyrimidine-2-carbaldehyde (55 mg, 0.2508 mmol) were dissolved/suspended in dichloromethane (1 mL). acetic acid (14 μL, 0.2462 mmol) was added. The mixture was cooled to 0° C. before the addition of DIEA (80 μL, 0.4593 mmol). After 5 minutes of stirring at 0° C., sodium triacetoxyborohydride (145 mg, 0.6842 mmol) was added, and stirring was continued at 0° C. for 30 minutes. The reaction mixture was quenched with the addition of aqueous 1 M HCl. After briefly stirring, it was diluted with EtOAc (50 mL) and washed with aqueous HCl (1 M, 1×50 mL). The organic layer was dried over sodium sulfate, filtered and concentrated under reduced pressure. The crude product was flash chromatographed on a 12 gram silica gel column eluting with a 0-15% methanol/dichloromethane gradient over 30 minutes. 3-[[4-[(2R)-4-cyclopropyl-2-[[5-(4,4-dimethyl-1-piperidyl)pyrimidin-2-yl]methylamino]butoxy]-6-(2,6-dimethylphenyl)pyrimidin-2-yl]sulfamoyl]benzoic acid (hydrochloride salt) (129 mg, 44%) was obtained as a light beige solid. ESI-MS m/z calc. 713.33594, found 714.5 (M+1)⁺; Retention time: 1.33 minutes; LC method A.

### Step 7: (11R)-11-(2-Cyclopropylethyl)-6-(2,6-dimethylphenyl)-12-[[5-(4,4-dimethyl-1-piperidyl)pyrimidin-2-yl]methyl]-2,2-dioxo-9-oxa-2⁶-thia-3,5,12,19-tetrazatricyclo[12.3.1.14,8]nonadeca-1(18),4(19),5,7,14,16-hexaen-13-one (Compound 1311)

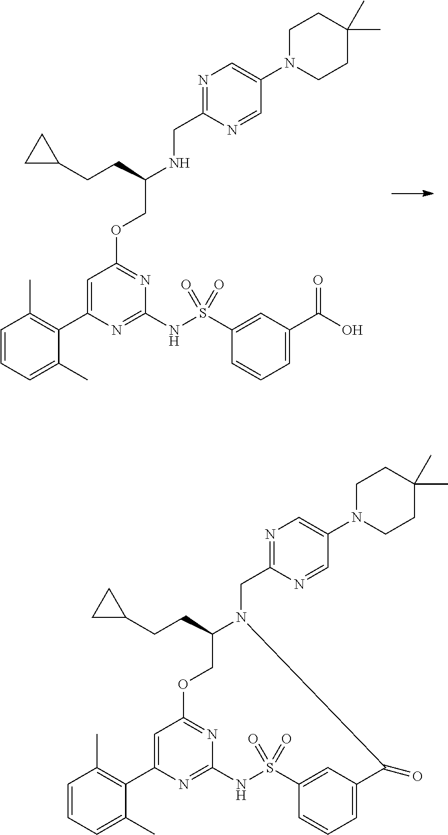 [SMILES: Cc1cccc(C)c1-c1cc(OC[C@@H](CCC2CC2)NCc2ncc(N3CCC(C)(C)CC3)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1ncc(N3CCC(C)(C)CC3)cn1)[C@H](CCC1CC1)CO2]

3-[[4-[(2R)-4-Cyclopropyl-2-[[5-(4,4-dimethyl-1-piperidyl)pyrimidin-2-yl]methylamino]butoxy]-6-(2,6-dimethylphenyl)pyrimidin-2-yl]sulfamoyl]benzoic acid (hydrochloride salt) (129 mg, 0.09971 mmol) and 2-chloro-4,6-dimethoxy-1,3,5-triazine (26 mg, 0.1481 mmol) were combined and dissolved in DMF (2.5 mL). The solution was cooled to 0° C. before the addition of N-methylmorpholine (55 μL, 0.5003 mmol). The reaction mixture was allowed to warm to room temperature and stirred overnight. After filtration, the product was isolated by UV-triggered reverse-phase HPLC eluting with a 10-99% acetonitrile/water gradient over 30 minutes with 5 mM HCl acid modifier in the aqueous phase. (11R)-11-(2-Cyclopropylethyl)-6-(2,6-dimethylphenyl)-12-[[5-(4,4-dimethyl-1-piperidyl)pyrimidin-2-yl]methyl]-2,2-dioxo-9-oxa-2?⁶-thia-3,5,12,19-tetrazatricyclo[12.3.1.14,8]nonadeca-1(18),4(19),5,7,14,16-hexaen-13-one (22.9 mg, 30%) was obtained. ¹H NMR (400 MHz, DMSO-d₆) δ 13.02 (s, 1H), 8.62 (s, 1H), 8.50 (s, 2H), 7.95-7.88 (m, 1H), 7.65 (d, J 4.9 Hz, 2H), 7.26 (t, J 7.6 Hz, 1H), 7.13 (d, J 7.6 Hz, 2H), 6.38 (s, 1H), 5.35 (dd, J 10.6, 3.9 Hz, 1H), 4.90 (d, J 16.3 Hz, 1H), 4.51 (d, J 16.4 Hz, 1H), 4.14 (t, J 11.1 Hz, 1H), 4.04 (td, J 11.1, 10.2, 5.3 Hz, 1H), 3.28-3.25 (m, 3H), 2.05 (s, 6H), 1.83 (ddt, J 13.7, 9.0, 4.4 Hz, 1H), 1.64 (dtd, J 10.6, 7.4, 3.8 Hz, 1H), 1.47-1.40 (m, 4H), 0.96 (s, 9H), 0.35-0.27 (m, 1H), 0.18-0.07 (m, 2H), −0.20-−0.29 (m, J 3.9 Hz, 2H). ESI-MS m/z calc. 695.3254, found 696.5 (M+1)⁺; Retention time: 1.83 minutes; LC method A.

### Example 154: Preparation of Compound 1312

### Step 1: Methyl 3-[[4-chloro-6-(2,6-dimethylphenyl)pyrimidin-2-yl]amino]sulfanylbenzoate

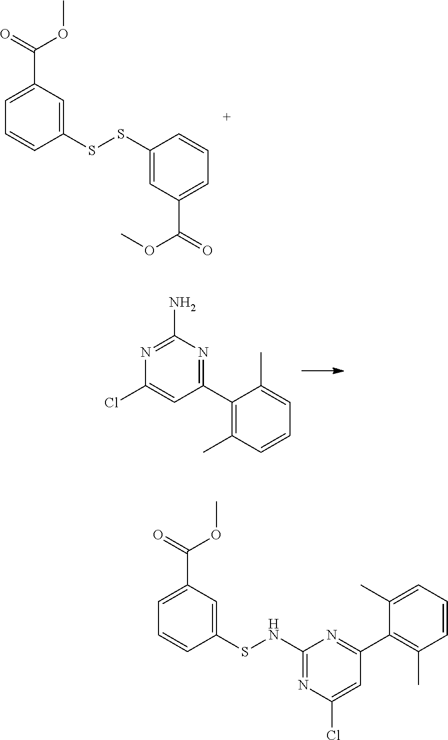 [SMILES: COC(=O)c1cccc(SSc2cccc(C(=O)OC)c2)c1 ; Cc1cccc(C)c1-c1cc(Cl)nc(N)n1 ; COC(=O)c1cccc(SNc2nc(Cl)cc(-c3c(C)cccc3C)n2)c1]

A 500 ml single necked round bottomed flask was charged, under an atmosphere of N₂, with methyl 3-[(3-methoxycarbonylphenyl)disulfanyl]benzoate (26.5 g, 79.244 mmol), dichloromethane (167 mL) and pyridine (2.9340 g, 3 mL, 37.092 mmol). To the resulting amber solution was added dropwise sulfuryl chloride (10.7 g, 79.277 mmol) (no exotherm observed). The solution turned deep orange and was stirred 10 minutes at room temperature. In another 2 L three necked round bottomed flask was charged, under an atmosphere of N₂, with 4-chloro-6-(2,6-dimethylphenyl)pyrimidin-2-amine (25.95 g, 111.04 mmol) and dichloromethane (618 mL). The resulting light-yellow solution was cooled to 2° C. (internal temperature) using an ice bath. Then, triethylamine (47.335 g, 65.2 mL, 467.78 mmol) was added dropwise, keeping the internal temperature below 10° C. Once this solution reached again 2° C., the first solution was added dropwise (exothermic), keeping the internal temperature below 10° C. The resulting pale orange suspension was stirred at 2° C. (ice bath not removed) during 1 h. The reaction was poured into 5% wt. aqueous solution of sodium bicarbonate (585 ml, 63 vol.). After phases separation, the aqueous phase was extracted with DCM (3×50 ml). The combined organic phases were dried over sodium sulfate, filtered, and concentrated to dryness to afford a crude product (76.54 g) as amber viscous oil. The oil was mixed with silica gel (80 g, 1 part vs crude) and DCM. The suspension was concentrated to dryness to afford a fine orange powder. This dry pack was loaded onto silica gel (460 g, 6 parts vs crude) packed with Heptane, in a fritted glass. Elution was started with Heptane/EtOAc (80/20) (1 L) followed by 70/30 (5 L). The filtrate (pale yellow) was concentrated to dryness. During the concentration, a fine white solid was formed. The solid was suspended into Heptane/EtOAc (95/5) (50 ml), was cooled in an ice bath and was filtered. The off-white solid was washed with cold Heptane/EtOAc (95/5) (50 ml) and was dried under high vacuum to furnish methyl 3-[[4-chloro-6-(2,6-dimethylphenyl)pyrimidin-2-yl]amino]sulfanylbenzoate (26.46 g, 83%) as white powder. ¹H NMR (400 MHz, CDCl₃) δ 7.90-7.87 (m, 1H), 7.84 (dt, J 7.3, 1.6 Hz, 1H), 7.46-7.33 (m, 2H), 7.22-7.12 (m, 1H), 7.09-6.99 (m, 3H), 6.80 (s, 1H), 3.90 (s, 3H), 2.05 (s, 6H). ESI-MS m/z calc. 399.0808, found 400.0 (M+1)⁺; Retention time: 2.018 minutes; LC method X.

### Step 2: Methyl 3-[4-chloro-6-(2,6-dimethylphenyl)pyrimidin-2-yl]sulfinamoylbenzoate

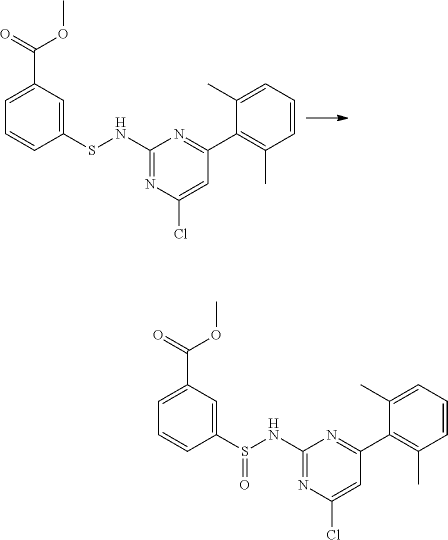 [SMILES: COC(=O)c1cccc(SNc2nc(Cl)cc(-c3c(C)cccc3C)n2)c1 ; COC(=O)c1cccc(S(=O)Nc2nc(Cl)cc(-c3c(C)cccc3C)n2)c1]

A 500 ml three necked round bottomed flask, equipped with an internal temperature probe, was charged, under an atmosphere of N₂, with methyl 3-[[4-chloro-6-(2,6-dimethylphenyl)pyrimidin-2-yl]amino]sulfanylbenzoate (26 g, 65.017 mmol) and dichloromethane (624 mL). The resulting pale-yellow solution was cooled to 2° C. (internal temperature) using an ice bath. Then, 3-chloroperbenzoic acid (15.6 g, 69.608 mmol) was added portion wise (light exotherm) keeping the internal temperature below 5° C. The resulting light-yellow suspension was stirred 1 h at 2° C. To the reaction mixture was added 5% wt. aqueous solution of Na₂S₂O₃ (520 ml, 20 vol.). An exotherm was observed and internal temperature reached 10° C. The mixture was poured into 5% wt. aqueous sodium bicarbonate (520 ml, 20 vol.). After phases separation, the aqueous phase was extracted with DCM (3×100 ml). Combined organic phases were dried over sodium sulfate, concentrated to dryness to afford crude product as yellow oil. The oil was mixed with Heptane/EtOAc (95/5) (200 ml) and was sonicated to obtain a white slurry. The slurry was stirred 30 minutes at room temperature. The solid was recovered by filtration, washed with cold Heptane/EtOAc (95/5) (100 ml) and dried under high vacuum to afford methyl 3-[4-chloro-6-(2,6-dimethylphenyl)pyrimidin-2-yl]sulfinamoylbenzoate (22.76 g, 84%) as white powder. ¹H NMR (400 MHz, CDCl₃) δ 8.48 (t, J 1.7 Hz, 1H), 8.25 (dt, J 7.7, 1.4 Hz, 1H), 8.07 (dt, J 7.8, 1.5 Hz, 1H), 7.67 (t, J 7.7 Hz, 1H), 7.35 (s, 1H), 7.26-7.21 (m, 1H), 7.12 (d, J 7.6 Hz, 2H), 6.96 (s, 1H), 3.96 (s, 3H), 2.15 (s, 6H). ESI-MS m/z calc. 415.07574, found 416.0 (M+1)⁺; Retention time: 1.906 minutes; LC method X.

### Step 3: Methyl 3-[[[4-chloro-6-(2,6-dimethylphenyl)pyrimidin-2-yl]amino]sulfonimidoyl]benzoate

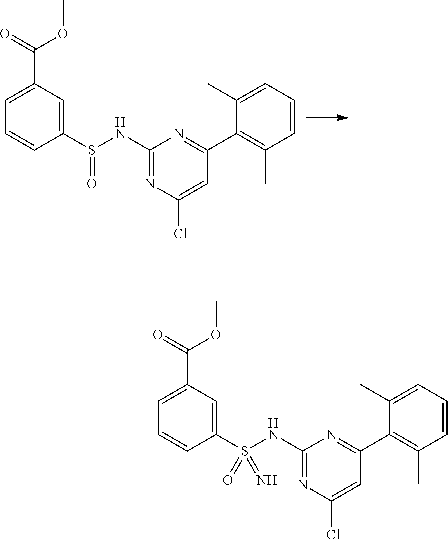 [SMILES: COC(=O)c1cccc(S(=O)Nc2nc(Cl)cc(-c3c(C)cccc3C)n2)c1 ; COC(=O)c1cccc(S(=N)(=O)Nc2nc(Cl)cc(-c3c(C)cccc3C)n2)c1]

A 3 L three necked round bottomed flask, equipped with a dropping funnel and an internal temperature probe, was charged, under an atmosphere of N₂, with methyl 3-[4-chloro-6-(2,6-dimethylphenyl)pyrimidin-2-yl]sulfinamoylbenzoate (22.5 g, 54.100 mmol) and dichloromethane (833 mL). To the resulting light-yellow solution was added portion wise 1-chloropyrrolidine-2,5-dione (10.11 g, 75.712 mmol). The milky white mixture was stirred at room temperature during 7 h. Then, the reaction was cooled to 0° C. (ice bath) and ammonia (0.4M in dioxane) (1.2 L of 0.4 M, 480.00 mmol) was added dropwise over 35 minutes. The reaction was stirred at room temperature overnight. The reaction was poured into a mixture of 5% wt. aqueous sodium bicarbonate/brine 1:1 (1 L). After phases separation, the aqueous phase was extracted with DCM (3×150 ml). Combined organic phases were washed with brine (250 ml), dried over sodium sulfate, filtered, and concentrated to dryness to afford crude product as yellow oil. The oil was solubilized in EtOAc and concentrated to dryness. A mixture of Hept/EtOAc 95/5 was added (a white solid appeared) and the solvents were concentrated to dryness. The solid was triturated with Hept/EtOAc 95/5 (200 ml). The white solid was recovered by filtration to afford methyl 3-[[[4-chloro-6-(2,6-dimethylphenyl)pyrimidin-2-yl]amino]sulfonimidoyl]benzoate (23.399 g, 81%) as an off-white powder containing about 15% wt of succinimide according to ¹H NMR. Methyl 3-[[[4-chloro-6-(2,6-dimethylphenyl)pyrimidin-2-yl]amino]sulfonimidoyl]benzoate (23.399 g, 49.577 mmol) was dissolved by sonication at 40° C. in EtOAc (250 mL). The organic phase was washed with sodium bicarbonate aqueous saturated (2×100 mL). The aqueous phase was backwashed with EtOAc (100 mL). The combined organic phases were washed with brine (100 mL), dried with magnesium sulfate, filtered and concentrated in vacuo to afford methyl 3-[[[4-chloro-6-(2,6-dimethylphenyl)pyrimidin-2-yl]amino]sulfonimidoyl]benzoate (21.98 g, 97%) as a pale yellow solid. ¹H NMR (400 MHz, CDCl₃) δ 8.69 (t, J 1.8 Hz, 1H), 8.28 (ddd, J 8.0, 1.9, 1.1 Hz, 1H), 8.23 (dt, J 7.9, 1.3 Hz, 1H), 7.56 (t, J 7.8 Hz, 1H), 7.21-7.15 (m, 1H), 7.04 (d, J 7.6 Hz, 2H), 6.74 (s, 1H), 6.00 (br. s., 2H), 3.90 (s, 3H), 2.02-1.84 (m, 6H). ESI-MS m/z calc. 430.08664, found 431.1 (M+1)⁺; Retention time: 3.975 minutes; LC method Y.

### Step 4: Methyl 3-[[[4-chloro-6-(2,6-dimethylphenyl)pyrimidin-2-yl]amino]sulfonimidoyl]benzoate, isomer A, and methyl 3-[[[4-chloro-6-(2,6-dimethylphenyl)pyrimidin-2-yl]amino]sulfonimidoyl]benzoate, isomer B

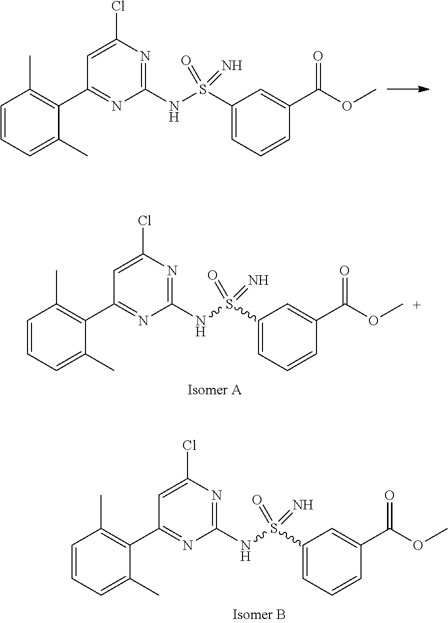

Racemic methyl 3-[[[4-chloro-6-(2,6-dimethylphenyl)pyrimidin-2-yl]amino]sulfonimidoyl]benzoate (21.98 g, 47.948 mmol) was dissolved in a 1:1 mixture of MeOH/MeCN (concentration of 1.2 g/25 mL) and submitted to chiral SFC separation (Flow rate: 75 mL/min, 15% MeOH, column: Cellulose 1, temperature=40° C., outlet pressure: 100 bar, injection volume: 600 μL). Fastest eluting peak by SFC gave after evaporation to dryness and co-evaporation with 2-methyltetrahydrofuran methyl 3-[[[4-chloro-6-(2,6-dimethylphenyl)pyrimidin-2-yl]amino]sulfonimidoyl]benzoate isomer A (8.38 g, 78%) as a pale yellow solid. ¹H NMR (400 MHz, DMSO-d₆) δ 8.39 (t, J 1.6 Hz, 1H), 8.15-8.07 (m, 2H), 7.89 (s, 2H), 7.67 (t, J 7.8 Hz, 1H), 7.22-7.15 (m, 1H), 7.03 (d, J 7.6 Hz, 2H), 6.91 (s, 1H), 3.83 (s, 3H), 1.93-1.51 (m, 6H). ESI-MS m/z calc. 430.0866, found 431.1 (M+1)⁺; Retention time: 3.99 minutes. The slowest eluting peak by SFC gave after evaporation to dryness and co-evaporation with 2-methyltetrahydrofuran methyl 3-[[[4-chloro-6-(2,6-dimethylphenyl)pyrimidin-2-yl]amino]sulfonimidoyl]benzoate isomer B (8.52 g, 76%) as a pale yellow solid. ¹H NMR (400 MHz, DMSO-d₆) δ 8.38 (t, J 1.6 Hz, 1H), 8.14-8.07 (m, 2H), 7.89 (s, 2H), 7.67 (t, J 7.8 Hz, 1H), 7.21-7.15 (m, 1H), 7.03 (d, J 7.6 Hz, 2H), 6.91 (s, 1H), 3.83 (s, 3H), 1.89-1.59 (m, 6H). ESI-MS m/z calc. 430.0866, found 431.1 (M+1)⁺; Retention time: 3.99 minutes; LC method Y.

### Step 5: 3-[[[4-Chloro-6-(2,6-dimethylphenyl)pyrimidin-2-yl]amino]sulfonimidoyl]benzoic acid isomer A

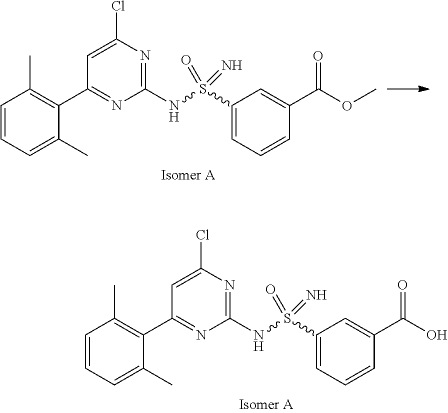

To a solution of methyl 3-[[[4-chloro-6-(2,6-dimethylphenyl)pyrimidin-2-yl]amino]sulfonimidoyl]benzoate isomer A (8.38 g, 18.125 mmol) in tetrahydrofuran (170 mL) and water (170 mL) at 0° C. was added lithium hydroxide hydrate (1.9 g, 45.277 mmol). The resulting light-yellow solution was stirred at room temperature for 16 h. The reaction mixture was diluted with aqueous saturated NH₄Cl (250 mL) and some HCl 1N to reach pH=4. The product was extracted with EtOAc (3×150 mL) and the combined organic phases were washed with brine (200 mL), dried with magnesium sulfate, filtered and concentrated to dryness to afford 3-[[[4-chloro-6-(2,6-dimethylphenyl)pyrimidin-2-yl]amino]sulfonimidoyl]benzoic acid isomer A (7.82 g, 96%) as a pale beige solid. ¹H NMR (400 MHz, DMSO-d₆) δ 8.44 (br. s., 1H), 8.11 (br. s., 1H), 7.93 (d, J 7.6 Hz, 1H), 7.72 (br. s., 2H), 7.53 (t, J 7.6 Hz, 1H), 7.21-7.11 (m, 1H), 7.02 (d, J 6.8 Hz, 2H), 6.88 (s, 1H), 1.77 (br. s., 6H). ESI-MS m/z calc. 416.07098, found 417.1 (M+1)⁺; Retention time: 3.57 minutes; LC method Y.

### Step 6: 3-[[[4-[(2R)-2-Amino-4,4-dimethyl-pentoxy]-6-(2,6-dimethylphenyl)pyrimidin-2-yl]amino]sulfonimidoyl]benzoic acid isomer A

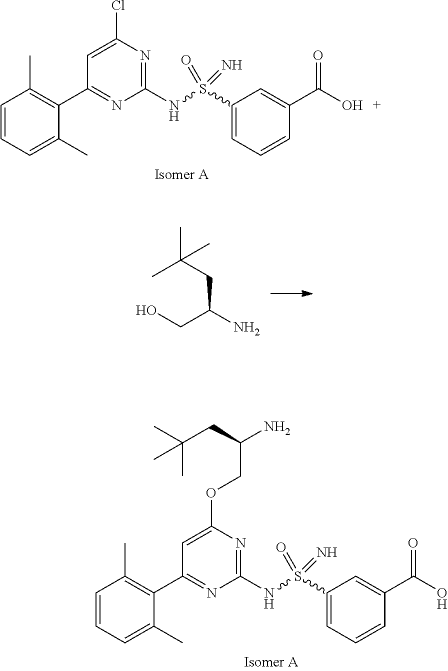

3-[[[4-Chloro-6-(2,6-dimethylphenyl)pyrimidin-2-yl]amino]sulfonimidoyl]benzoic acid isomer A (4 g, 8.5012 mmol) was dissolved in 2-MeTHF (36 mL) and DMF (4 mL). The reaction mixture was cooled to 0° C. and sodium tert-butoxide (4.8 g, 49.946 mmol) was added followed by (2R)-2-amino-4,4-dimethyl-pentan-1-ol (hydrochloride salt) (1.8 g, 10.735 mmol). The reaction was then warmed to room temperature and stirred for 5.5 h. More sodium tert-butoxide (817 mg, 8.5013 mmol) was added and the reaction mixture was stirred 15 min. at room temperature. The reaction was cooled to 0° C. and quenched by adding an aqueous solution of hydrochloric acid (2M, 60 mL). The reaction mixture stood still overnight at room temperature and then the mixture was evaporated to dryness and the residue was purified twice by reverse phase chromatography on a 120 g C₁₈Aq cartridge using a gradient of 10-100% MeCN in acidic water (0.1% HCl) to afford after lyophilization 3-[[[4-[(2R)-2-amino-4,4-dimethyl-pentoxy]-6-(2,6-dimethylphenyl)pyrimidin-2-yl]amino]sulfonimidoyl]benzoic acid (hydrochloride salt) isomer A (2.24 g, 46%) as a pale beige solid. ¹H NMR (400 MHz, DMSO-d₆) δ 8.62-8.31 (m, 5H), 8.30-8.21 (m, 2H), 7.83 (t, J=7.8 Hz, 1H), 7.38-7.30 (m, 1H), 7.19 (d, J 7.6 Hz, 2H), 6.55 (br. s., 1H), 4.48 (d, J 12.0 Hz, 1H), 3.80 (dd, J 11.6, 7.5 Hz, 1H), 3.53 (br. s., 1H), 2.08 (br. s, 6H), 1.52 (d, J 5.6 Hz, 2H), 0.92 (s, 9H). ESI-MS m/z calc. 511.2253, found 512.2 (M+1)⁺; Retention time: 2.11 minutes; LC method Y.

### Step 7: 3-[[[4-[(2R)-2-[[5-(3,3-Dimethylazetidin-1-yl)pyrimidin-2-yl]methylamino]-4,4-dimethyl-pentoxy]-6-(2,6-dimethylphenyl)pyrimidin-2-yl]amino]sulfonimidoyl]benzoic acid isomer A

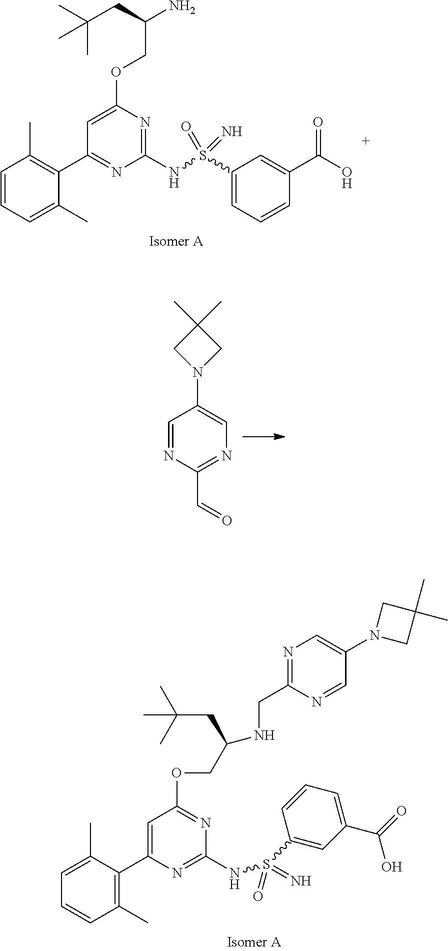

To a stirred mixture of 3-[[[4-[(2R)-2-amino-4,4-dimethyl-pentoxy]-6-(2,6-dimethylphenyl)pyrimidin-2-yl]amino]sulfonimidoyl]benzoic acid (hydrochloride salt) isomer A (80 mg, 0.1460 mmol) and 5-(3,3-dimethylazetidin-1-yl)pyrimidine-2-carbaldehyde (28 mg, 0.1464 mmol) in anhydrous dichloromethane (0.6 mL) were added glacial acetic acid (15 μL, 0.2638 mmol) and N,N-diisopropylethylamine (80 μL, 0.4593 mmol), in that order, at 0° C. (ice-water bath). The yellow solution was stirred for 2-3 min, then sodium triacetoxyborohydride (94 mg, 0.4435 mmol) was added at once at the same temperature. After stirring for another 5 min, the reaction mixture was poured over ice-water (20 mL) containing hydrochloric acid (2 mL of 1 M, 2.000 mmol). The product was extracted with ethyl acetate (3×30 mL) and the combined organics were washed with water (20 mL), brine (20 mL), dried over sodium sulfate, filtered, and concentrated under reduced pressure. The crude was purified by preparative reverse-phase HPLC (1-99% acetonitrile in water over 15 min, HCl as modifier). Then immediately, the desired fractions were combined and extracted with ethyl acetate (3×30 mL) and the combined organics successively were washed with water (20 mL), brine (20 mL), dried over sodium sulfate, filtered and concentrated under reduced pressure to furnish 3-[[[4-[(2R)-2-[[5-(3,3-dimethylazetidin-1-yl)pyrimidin-2-yl]methylamino]-4,4-dimethyl-pentoxy]-6-(2,6-dimethylphenyl)pyrimidin-2-yl]amino]sulfonimidoyl]benzoic acid isomer A (75 mg, 75%) as a white solid. It was used in the cyclization step. ESI-MS m/z calc. 686.33624, found 687.3 (M+1)⁺; Retention time: 1.28 minutes; LC method A.

### Step 8: (11R)-2-Amino-12-[[5-(3,3-dimethylazetidin-1-yl)pyrimidin-2-yl]methyl]-6-(2,6-dimethylphenyl)-11-(2,2-dimethylpropyl)-2-oxo-9-oxa-2λ⁶-thia-3,5,12,19-tetrazatricyclo[12.3.1.14,8]nonadeca-1(18),2,4(19),5,7,14,16-heptaen-13-one, isomer A (Compound 1312)

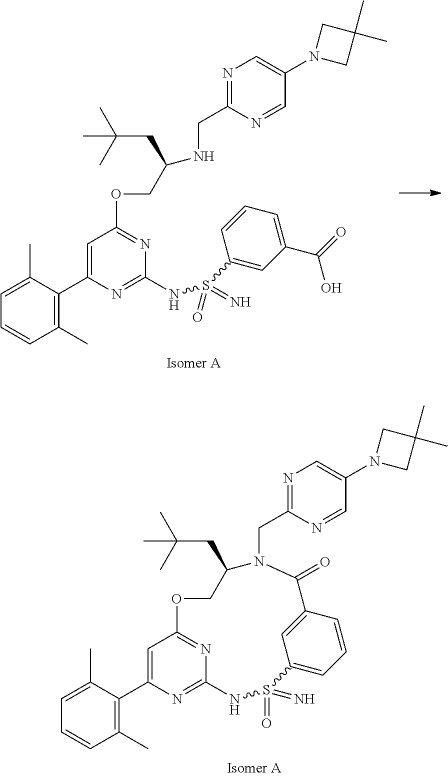

To a stirred solution of 3-[[[4-[(2R)-2-[[5-(3,3-dimethylazetidin-1-yl)pyrimidin-2-yl]methylamino]-4,4-dimethyl-pentoxy]-6-(2,6-dimethylphenyl)pyrimidin-2-yl]amino]sulfonimidoyl]benzoic acid isomer A (75 mg, 0.1092 mmol) in anhydrous N,N-dimethylformamide (5 mL) was added 2-chloro-4,6-dimethoxy-1,3,5-triazine (30 mg, 0.1709 mmol) (CDMT), followed by addition of 4-methylmorpholine (80 μL, 0.7277 mmol) at 0-4° C. (ice-water bath) under nitrogen. The clear reaction was allowed to stir at that temperature for 5 min, then allowed to stir at room temperature. Within 15 min, then reaction became turbid. After stirring for 14 h (overnight), the volatiles were removed under reduced pressure and the residue was purified by silica gel chromatography (0-10% methanol in dichloromethane over 30 min, and the product peak came around 7% methanol). The product was purified further by preparative reverse-phase HPLC (1-99% acetonitrile in water over 15 min, HCl as modifier). The desired fraction was extracted with ethyl acetate (3×50 mL). The organics were washed with brine (2×30 mL), dried over sodium sulfate, filtered, concentrated under reduced pressure and dried in vacuo to furnish slightly impure material again. Then second rounds of silica gel then reverse-phase HPLC, followed by above ethyl acetate workup furnished (11R)-2-amino-12-[[5-(3,3-dimethylazetidin-1-yl)pyrimidin-2-yl]methyl]-6-(2,6-dimethylphenyl)-11-(2,2-dimethylpropyl)-2-oxo-9-oxa-2?⁶-thia-3,5,12,19-tetrazatricyclo[12.3.1.14,8]nonadeca-1(18),2,4(19),5,7,14,16-heptaen-13-one isomer A (7 mg, 9%) as a white solid. ¹H NMR (400 MHz, DMSO-d₆) δ 8.70 (d, J 1.9 Hz, 1H), 8.09 (d, J 8.2 Hz, 1H), 8.03 (s, 2H), 7.94 (s, 2H), 7.71 (t, J 7.7 Hz, 1H), 7.61 (d, J 7.6 Hz, 1H), 7.14 (t, J 7.5 Hz, 1H), 7.04 (d, J 7.6 Hz, 2H), 6.15 (s, 1H), 5.31 (dd, J 10.1, 3.4 Hz, 1H), 4.83 (d, J 16.4 Hz, 1H), 4.52 (d, J 16.4 Hz, 1H), 3.92-3.73 (m, 2H), 3.64 (s, 4H), 1.92 (s, 6H), 1.74 (dd, J 15.1, 8.5 Hz, 1H), 1.42 (d, J 14.9 Hz, 1H), 1.29 (s, 6H), 0.55 (s, 9H). ESI-MS m/z calc. 668.3257, found 669.3 (M+1)⁺; Retention time: 1.62 minutes; LC method A.

### Example 155: Preparation of Compound 1313

### Step 1: 3-[[[4-Chloro-6-(2,6-dimethylphenyl)pyrimidin-2-yl]amino]sulfonimidoyl]benzoic acid isomer B

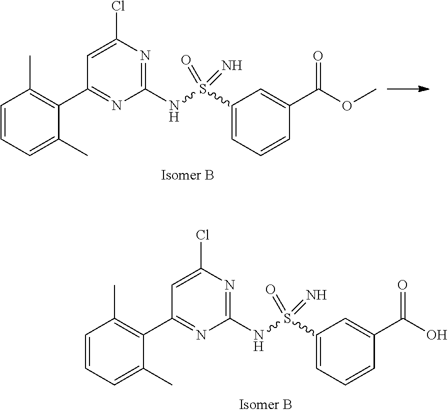

To a solution of methyl 3-[[[4-chloro-6-(2,6-dimethylphenyl)pyrimidin-2-yl]amino]sulfonimidoyl]benzoate isomer B (8.52 g, 17.637 mmol) in tetrahydrofuran (170 mL) and water (170 mL) at 0° C. was added lithium hydroxide hydrate (1.85 g, 44.086 mmol). The resulting light-yellow solution was stirred at room temperature for 16 h. The reaction mixture was diluted with aqueous saturated NH₄Cl (200 mL) and some HCl 1N (approx. 30 mL) to reach pH=4. The product was extracted with EtOAc (3×200 mL) and the combined organic phases were washed with brine (200 mL), dried with magnesium sulfate, filtered and concentrated to dryness to afford 3-[[[4-chloro-6-(2,6-dimethylphenyl)pyrimidin-2-yl]amino]sulfonimidoyl]benzoic acid isomer B (7.62 g, 96%) as a beige solid. ¹H NMR (400 MHz, DMSO-d₆) δ 8.42 (br. s., 1H), 8.10 (d, J 6.8 Hz, 1H), 7.95 (d, J 7.8 Hz, 1H), 7.74 (br. s., 2H), 7.55 (t, J 7.7 Hz, 1H), 7.20-7.13 (m, 1H), 7.03 (d, J 7.3 Hz, 2H), 6.89 (s, 1H), 1.95-1.56 (m, 6H). ESI-MS m/z calc. 416.07098, found 417.1 (M+1)⁺; Retention time: 3.58 minutes; LC method Y.

### Step 2: 3-[[[4-[(2R)-2-Amino-4,4-dimethyl-pentoxy]-6-(2,6-dimethylphenyl)pyrimidin-2-yl]amino]sulfonimidoyl]benzoic acid isomer B

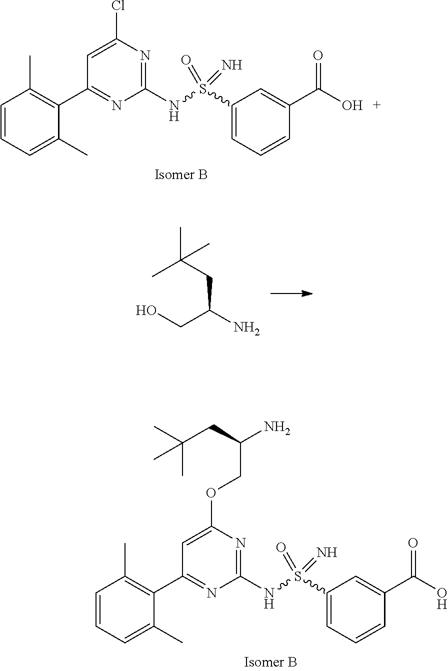

3-[[[4-Chloro-6-(2,6-dimethylphenyl)pyrimidin-2-yl]amino]sulfonimidoyl]benzoic acid isomer B (3.97 g, 8.4279 mmol) was dissolved in 2-MeTHF (36 mL) and DMF (4 mL). The reaction mixture was cooled to 0° C. and sodium tert-butoxide (4.05 g, 42.142 mmol) was added followed by (2R)-2-amino-4,4-dimethyl-pentan-1-ol (hydrochloride salt) (1.7 g, 10.139 mmol). The reaction was then warmed to room temperature and stirred for 6 h. More sodium tert-butoxide (2 g, 20.811 mmol) was added and the mixture was stirred 18 h at room temperature. The reaction was cooled to 0° C. and quenched by adding an aqueous solution of hydrochloric acid (2M, 70 mL). The mixture was evaporated to dryness and the residue was purified twice by reverse phase chromatography on a 120 g C₁₈Aq cartridge using a gradient of 5-100% MeCN in acidic water (0.1% HCl) to afford after lyophilization 3-[[[4-[(2R)-2-amino-4,4-dimethyl-pentoxy]-6-(2,6-dimethylphenyl)pyrimidin-2-yl]amino]sulfonimidoyl]benzoic acid (hydrochloride salt) isomer B (2.13 g, 45%) as an off-white solid. ¹H NMR (400 MHz, DMSO-d₆) δ 8.55-8.33 (m, 5H), 8.29-8.20 (m, 2H), 7.82 (t, J=7.8 Hz, 1H), 7.36-7.29 (m, 1H), 7.18 (d, J 7.6 Hz, 2H), 6.53 (br. s., 1H), 4.32 (dd, J 11.7, 7.6 Hz, 1H), 3.96 (d, J 11.5 Hz, 1H), 3.49 (br. s., 1H), 2.07 (br. s., 6H), 1.58-1.47 (m, 2H), 0.93 (s, 9H). ESI-MS m/z calc. 511.2253, found 512.3 (M+1)⁺; Retention time: 2.13 minutes; LC method Y.

### Step 3: 3-[[[4-[(2R)-2-[[5-(3,3-Dimethylazetidin-1-yl)pyrimidin-2-yl]methylamino]-4,4-dimethyl-pentoxy]-6-(2,6-dimethylphenyl)pyrimidin-2-yl]amino]sulfonimidoyl]benzoic acid isomer B

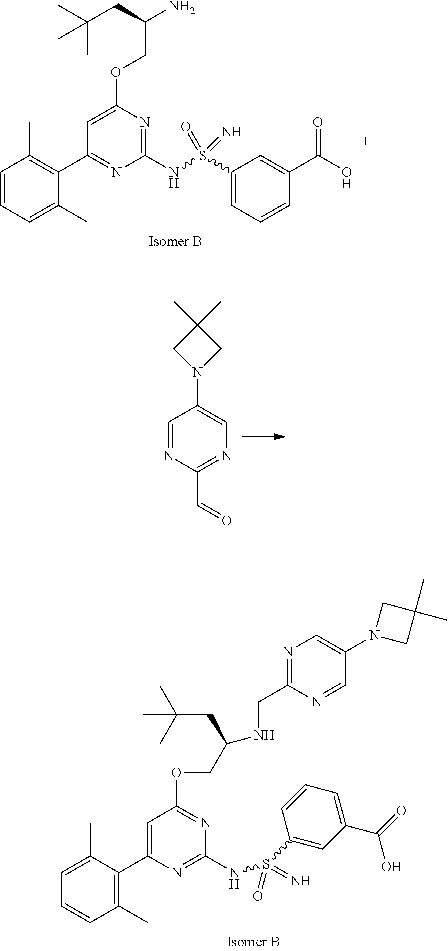

To a stirred mixture of 3-[[[4-[(2R)-2-amino-4,4-dimethyl-pentoxy]-6-(2,6-dimethylphenyl)pyrimidin-2-yl]amino]sulfonimidoyl]benzoic acid (hydrochloride salt) isomer B (80 mg, 0.1460 mmol) and 5-(3,3-dimethylazetidin-1-yl)pyrimidine-2-carbaldehyde (28 mg, 0.1464 mmol) in anhydrous dichloromethane (0.6 mL) were added glacial acetic acid (15 μL, 0.2638 mmol) and N,N-diisopropylethylamine (80 μL, 0.4593 mmol), in that order, at 0° C. (ice-water bath). The yellow solution was stirred for 2-3 min, then sodium triacetoxyborohydride (95 mg, 0.4482 mmol) was added at once at the same temperature. After stirring for another 5 min, the reaction mixture was poured over ice-water (20 mL) containing hydrochloric acid (2 mL of 1 M, 2.000 mmol). The product was extracted with ethyl acetate (3×30 mL) and the combined organics were washed with water (20 mL), brine (20 mL), dried over sodium sulfate, filtered and concentrated under reduced pressure. The crude was purified by preparative reverse-phase HPLC (1-99% acetonitrile in water over 15 min, HCl as modifier). Immediately the desired fractions were combined and extracted with ethyl acetate (3×30 mL) and the combined organics successively were washed with water (20 mL), brine (20 mL), dried over sodium sulfate, filtered and concentrated under reduced pressure to furnish 3-[[[4-[(2R)-2-[[5-(3,3-dimethylazetidin-1-yl)pyrimidin-2-yl]methylamino]-4,4-dimethyl-pentoxy]-6-(2,6-dimethylphenyl)pyrimidin-2-yl]amino]sulfonimidoyl]benzoic acid (hydrochloride salt) isomer B (102 mg, 97%) as a white solid. It was used in the cyclization step. ESI-MS m/z calc. 686.33624, found 687.3 (M+1)⁺; Retention time: 1.24 minutes; LC method A.

### Step 4: (11R)-2-Amino-12-[[5-(3,3-dimethylazetidin-1-yl)pyrimidin-2-yl]methyl]-6-(2,6-dimethylphenyl)-11-(2,2-dimethylpropyl)-2-oxo-9-oxa-2λ⁶-thia-3,5,12,19-tetrazatricyclo[12.3.1.14,8]nonadeca-1(18),2,4(19),5,7,14,16-heptaen-13-one isomer B (Compound 1313)

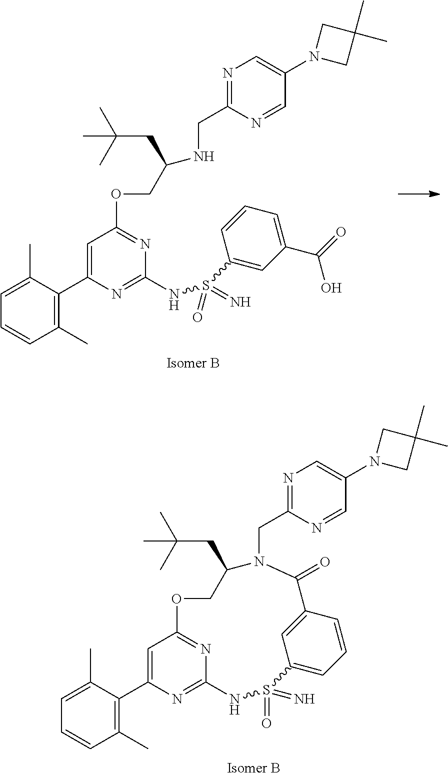

To a stirred solution of 3-[[[4-[(2R)-2-[[5-(3,3-dimethylazetidin-1-yl)pyrimidin-2-yl]methylamino]-4,4-dimethyl-pentoxy]-6-(2,6-dimethylphenyl)pyrimidin-2-yl]amino]sulfonimidoyl]benzoic acid (hydrochloride salt) isomer B (80 mg, 0.1106 mmol) in anhydrous N,N-dimethylformamide (6 mL) was added 2-chloro-4,6-dimethoxy-1,3,5-triazine (30 mg, 0.1709 mmol) (CDMT), followed by addition 4-methylmorpholine (80 μL, 0.7277 mmol) of at 0-4° C. (ice-water bath) under nitrogen. The clear reaction was allowed to stir at that temperature for 5 min, then allowed to stir at room temperature. Within 15 min, then reaction became turbid. After stirring for 14 h (overnight), the volatiles were removed under reduced pressure and the residue was purified by silica gel chromatography (0-10% methanol in dichloromethane over 30 min, and the product peak came around 7% methanol). The product was purified further by preparative reverse-phase HPLC (1-99% acetonitrile in water over 15 min, HCl as modifier). The desired fraction was extracted with ethyl acetate (3×50 mL). The organics were washed with brine (2×30 mL), dried over sodium sulfate, filtered, concentrated under reduced pressure, and dried in vacuo to furnish slightly impure material again. Then a second round of silica gel then reverse-phase HPLC, followed by above ethyl acetate workup furnished (11R)-2-amino-12-[[5-(3,3-dimethylazetidin-1-yl)pyrimidin-2-yl]methyl]-6-(2,6-dimethylphenyl)-11-(2,2-dimethylpropyl)-2-oxo-9-oxa-2λ⁶-thia-3,5,12,19-tetrazatricyclo[12.3.1.14,8]nonadeca-1(18),2,4(19),5,7,14,16-heptaen-13-one isomer B (2.8 mg, 4%) as a white solid. ESI-MS m/z calc. 668.3257, found 669.0 (M+1)⁺; Retention time: 1.61 minutes; LC method A.

### Example 156: Preparation of Compound 1314

### Step 1: (11R)-12-[[6-(2-Azabicyclo[3.2.0]heptan-2-yl)pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

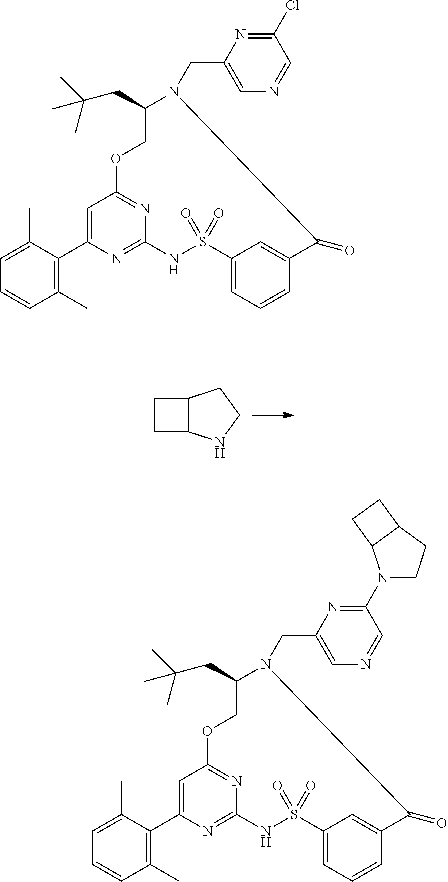 [SMILES: Cc1cccc(C)c1-c1cc2nc(n1)NS(=O)(=O)c1cccc(c1)C(=O)N(Cc1cncc(Cl)n1)[C@H](CC(C)(C)C)CO2 ; C1CC2CCC2N1 ; Cc1cccc(C)c1-c1cc2nc(n1)NS(=O)(=O)c1cccc(c1)C(=O)N(Cc1cncc(N3CCC4CCC43)n1)[C@H](CC(C)(C)C)CO2]

(11R)-12-[(6-Chloropyrazin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (40 mg, 0.06440 mmol) was combined with 2-azabicyclo[3.2.0]heptane (25 mg, 0.2573 mmol) and freshly ground potassium carbonate (75 mg, 0.5427 mmol) in a screwcap vial with an unpierced septum. DMSO (300 μL) and dioxane (0.1 mL) were added and the reaction was heated to 120° C. overnight. The reaction was then cooled to room temperature, diluted with methanol, filtered, and purified by reverse phase preparative HPLC (1-99% ACN in water, HCl modifier) to give (11R)-12-[[6-(2-azabicyclo[3.2.0]heptan-2-yl)pyrazin-2-yl]methyl]-6-(2,6-dimethylphenyl)-11-(2,2-dimethylpropyl)-2,2-dioxo-9-oxa-2λ⁶-thia-3, 5,12,19-tetrazatricyclo[12.3.1.14,8]nonadeca-1(18),4(19),5,7,14,16-hexaen-13-one (30.4 mg, 69%). ESI-MS m/z calc. 681.30975, found 682.8 (M+1)⁺; Retention time: 0.71 minutes; LC method D. Step 2: (11R)-12-[[6-(2-Azabicyclo[3.2.0]heptan-2-yl)pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1, and (11R)-12-[[6-(2-azabicyclo[3.2.0]heptan-2-yl)pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2, (Compound 1314)

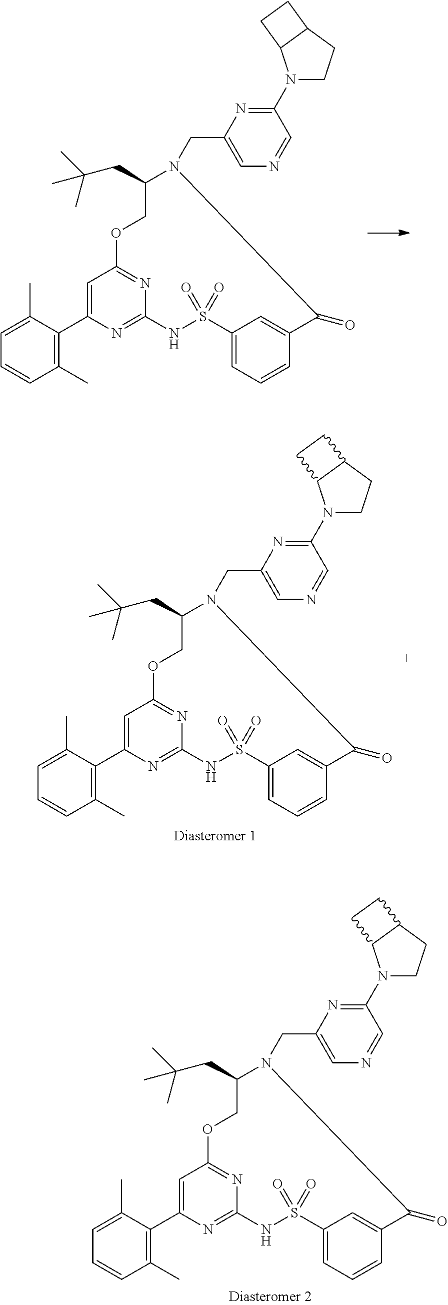 [SMILES: Cc1cccc(C)c1-c1cc2nc(n1)NS(=O)(=O)c1cccc(c1)C(=O)N(Cc1cncc(N3CCC4CCC43)n1)[C@H](CC(C)(C)C)CO2 ; Cc1cccc(C)c1-c1cc2nc(n1)NS(=O)(=O)c1cccc(c1)C(=O)N(Cc1cncc(N3CCC4CCC43)n1)[C@H](CC(C)(C)C)CO2 ; Cc1cccc(C)c1-c1cc2nc(n1)NS(=O)(=O)c1cccc(c1)C(=O)N(Cc1cncc(N3CCC4CCC43)n1)[C@H](CC(C)(C)C)CO2]

The diastereomers of (11R)-12-[[6-(2-azabicyclo[3.2.0]heptan-2-yl)pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (30 mg, 0.04400 mmol) were separated by chiral SFC using a ChiralPak AS column (250×10 mm; 5 m) at 50° C. Mobile phase was 32% MeOH (20 mM NH₃), 68% CO₂ at a 20 mL/min flow. Concentration of the sample was 15 mg/mL in MeOH (No Modifier), injection volume 100 μL with an outlet pressure of 209 bar, detection wavelength of 210 nm. Each diastereomer was collected separately and the resulting products were each further purified by chromatography on silica gel eluting with a 0-15% DCM in methanol gradient. An insufficient quantity of the first peak for submission was obtained (Diastereomer 1). Diastereomer 2, the second eluting peak was obtained in a larger quantity, (11R)-12-[[6-(2-azabicyclo[3.2.0]heptan-2-yl)pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (6.3 mg, 21%). ¹H NMR (400 MHz, DMSO-d₆) δ 13.00 (s, 1H), 8.76 (s, 1H), 7.94 (s, 1H), 7.87 (s, 1H), 7.77 (s, 1H), 7.67 (s, 2H), 7.24 (d, J 7.9 Hz, 1H), 7.12 (d, J 7.6 Hz, 2H), 6.42 (s, 1H), 5.51 (d, J 9.8 Hz, 1H), 4.73 (d, J 16.1 Hz, 1H), 4.54 (s, 1H), 4.46 (d, J 16.1 Hz, 1H), 4.30-4.18 (m, 1H), 4.07-3.94 (m, 2H), 3.66 (q, J 9.8, 9.2 Hz, 1H), 3.23-3.16 (m, 1H), 2.45-2.38 (m, 1H), 2.17-1.90 (m, 8H), 1.85 (dd, J 13.0, 6.9 Hz, 1H), 1.72 (dd, J 15.9, 8.8 Hz, 2H), 1.63 (d, J 12.6 Hz, 1H), 1.42 (d, J 15.0 Hz, 1H), 0.57 (s, 9H). ESI-MS m/z calc. 681.30975, found 682.9 (M+1)⁺; Retention time: 1.9 minutes; LC method A.

### Example 157: Preparation of Compound 1315 and Compound 1316

### Step 1: (11R)-12-[[6-(2-Azabicyclo[4.2.0]octan-2-yl)pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 1315) and (11R)-12-[[6-(2-azabicyclo[4.2.0]octan-2-yl)pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 1316)

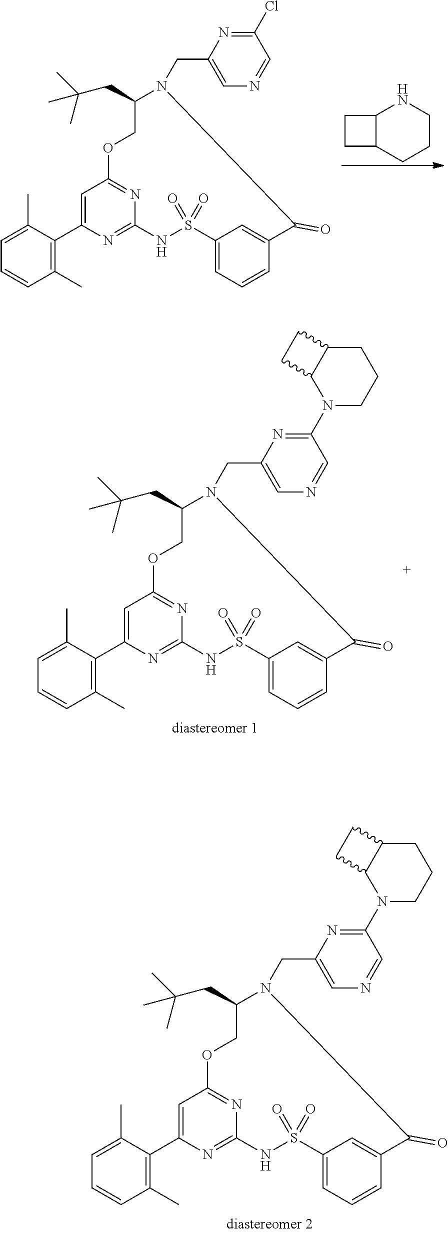 [SMILES: Cc1cccc(C)c1-c1cc2nc(n1)NS(=O)(=O)c1cccc(c1)C(=O)N(Cc1cncc(Cl)n1)[C@H](CC(C)(C)C)CO2 ; C1CNC2CCC2C1 ; Cc1cccc(C)c1-c1cc2nc(n1)NS(=O)(=O)c1cccc(c1)C(=O)N(Cc1cncc(N3CCCC4CCC43)n1)[C@H](CC(C)(C)C)CO2 ; Cc1cccc(C)c1-c1cc2nc(n1)NS(=O)(=O)c1cccc(c1)C(=O)N(Cc1cncc(N3CCCC4CCC43)n1)[C@H](CC(C)(C)C)CO2]

A 4 mL vial was charged with (11R)-12-[(6-chloropyrazin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (47 mg, 0.07567 mmol), (1S,6R)-2-azabicyclo[4.2.0]octane (hydrochloride salt) (31 mg, 0.2100 mmol) (racemic cis isomer), carbonate (Potassium Ion (2)) (88 mg, 0.6367 mmol) (powdered, 325 mesh), DMSO (200 μL) and dioxane (100 μL). The vial was purged with nitrogen, capped, and stirred at 120° C. for 18 h. After cooling down to room temperature, the reaction was diluted with DMSO (1 mL), filtered and purified by reverse phase HPLC (1-99% acetonitrile/5 mM aqueous HCl over 15 min) to give (11R)-12-[[6-(2-azabicyclo[4.2.0]octan-2-yl)pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (47 mg, 89%) as a white solid mixture of diastereomers. ESI-MS m/z calc. 695.3254, found 696.81 (M+1)⁺; Retention time: 1.99 minutes, second isomer, found 696.87 (M+1)⁺, retention time: 2.01 minutes (approximatively 1:1 ratio). The diastereomers were separated by chiral SFC using a ChiralPak AS column (250×21.2 mm; 5 m) at 50° C. Mobile phase was 32% MeOH (20 mM NH₃), 68% CO₂ at a 70 mL/min flow. Concentration of the sample was 18.7 mg/mL in MeOH (no modifier), injection volume 420 μL with an outlet pressure of 201 bar, detection wavelength of 210 nm. For each isomer, the solvents were evaporated, the residue was dissolved in DCM and purified by flash chromatography on silica gel (4 g column) using a gradient of methanol (0 to 15% over 15 min) in dichloromethane. Trituration in DCM/hexanes and evaporation of the solvents gave both isomers as white solids: SFC peak 1, diastereomer 1, (11R)-12-[[6-(2-azabicyclo[4.2.0]octan-2-yl)pyrazin-2-yl]methyl]-6-(2,6-dimethylphenyl)-11-(2,2-dimethylpropyl)-2,2-dioxo-9-oxa-2?⁶-thia-3,5,12,19-tetrazatricyclo[12.3.1.14,8]nonadeca-1(18),4(19),5,7,14,16-hexaen-13-one (11 mg, 42%). ¹H NMR (400 MHz, DMSO-d₆) δ 13.41-11.42 (broad m, 1H), 8.75 (s, 1H), 7.99-7.82 (m, 3H), 7.68 (br s, 2H), 7.25 (t, J 7.6 Hz, 1H), 7.12 (d, J 7.6 Hz, 2H), 6.43 (br s, 1H), 5.40-5.25 (m, 1H), 4.78 (d, J 16.3 Hz, 1H), 4.60-4.40 (m, 2H), 4.29-4.15 (m, 1H), 4.14-4.00 (m, 1H), 3.94-3.80 (m, 1H), 3.25-3.04 (m, 1H), 2.64-2.55 (m, 1H), 2.26-1.81 (m, 11H), 1.75 (dd, J 15.2, 8.9 Hz, 1H), 1.62-1.48 (m, 1H), 1.47-1.31 (m, 3H), 0.55 (s, 9H). ESI-MS m/z calc. 695.3254, found 696.76 (M+1)⁺; Retention time: 1.98 minutes; and SFC peak 2, diastereomer 2, (11R)-12-[[6-(2-azabicyclo[4.2.0]octan-2-yl)pyrazin-2-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (10 mg, 38%). ¹H NMR (400 MHz, DMSO-d₆) δ 13.62-11.53 (broad m, 1H), 8.70 (s, 1H), 8.01-7.85 (m, 3H), 7.67 (br s, 2H), 7.33-7.19 (m, 1H), 7.17-7.03 (m, 2H), 6.44 (br s, 1H), 5.52-5.35 (m, 1H), 4.70 (d, J 16.0 Hz, 1H), 4.58-4.40 (m, 2H), 4.31 (t, J 11.2 Hz, 1H), 4.09-3.98 (m, 1H), 3.93 (d, J 12.7 Hz, 1H), 3.17 (t, J 10.8 Hz, 1H), 2.63 (s, 1H), 2.30-1.83 (m, 11H), 1.74 (dd, J=15.2, 9.0 Hz, 1H), 1.60-1.38 (m, 4H), 0.56 (s, 9H). ESI-MS m/z calc. 695.3254, found 696.81 (M+1)⁺; Retention time: 1.99 minutes; LC method A.

### Example 158: Preparation of Compound 1317

### Step 1: N-Methoxy-N,1-dimethyl-cyclobutanecarboxamide

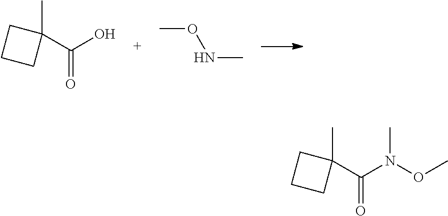

To a solution of 1-methylcyclobutanecarboxylic acid (18 g, 157.70 mmol) in DMF (200 mL) at 0° C. was added N-methoxymethanamine hydrochloride (31 g, 317.81 mmol) followed by HATU (70 g, 184.10 mmol) and triethylamine (50.820 g, 70 mL, 502.22 mmol). The mixture was stirred at 0° C. for 30 minutes and then at room temperature for 18 hours. Water (400 mL) and EtOAc (400 mL) were added, and the mixture was extracted with EtOAc (3×200 mL), washed with an aqueous 1N HCl solution (2×400 mL), an aqueous saturated solution of sodium bicarbonate (2×400 mL), water (2×400 mL) and brine (2×400 mL), dried over sodium sulfate, filtered and concentrated in vacuo to afford N-methoxy-N, 1-dimethyl-cyclobutanecarboxamide (20.5 g, 75%) as a yellow oil. ESI-MS m/z calc. 157.11028, found 158.4 (M+1)⁺; Retention time: 1.49 minutes; LC method X.

### Step 2: 1-Methyicyclobutanecarbaldehyde

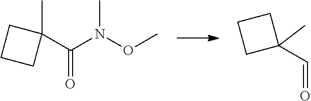

A solution of N-methoxy-N, 1-dimethyl-cyclobutanecarboxamide (20 g, 115.01 mmol) in dry dioxane (100 mL) was added to a suspension of LAH (6.5 g, 171.26 mmol) in dry dioxane (200 mL) at 0° C. The mixture was stirred at 0° C. for 5 minutes and then at room temperature for 2 hours. The mixture was then cooled down to 0° C. and water (6.5 mL) was added followed by an aqueous solution of NaOH (15%, 6.5 mL) and then water (19.5 mL). The mixture was stirred at room temperature for 30 minutes and magnesium sulfate was added (10 g). The mixture was filtered on Celite and the filter cake was rinsed with dioxane (100 mL) to afford 1-methylcyclobutanecarbaldehyde (11.28 g, 100%) in a dioxane solution. This solution will be used directly as is in the next reaction as a dioxane solution.

### Step 3: Methyl 2-(tert-butoxycarbonylamino)-3-(1-methylcyclobutyl)prop-2-enoate

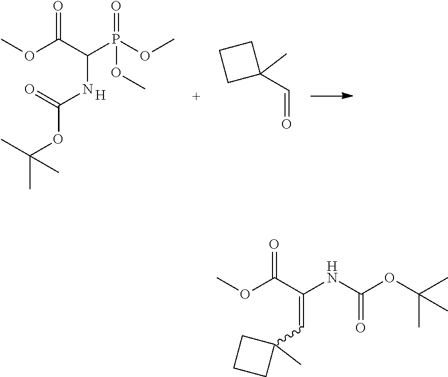

To a stirred solution of 1-methylcyclobutanecarbaldehyde (in a dioxane solution) (11.28 g, 114.93 mmol) at 0° C. was added methyl 2-(tert-butoxycarbonylamino)-2-dimethoxyphosphoryl-acetate (11.5 g, 38.689 mmol) followed by 1,1,3,3-tetramethylguanidine (13.311 g, 14.5 mL, 115.57 mmol). The reaction mixture was stirred at 0° C. for 1 hour and then at room temperature for 24 hours. Water (100 mL) and EtOAc (250 mL) were added, and the mixture was extracted with EtOAc (3×250 mL). The combined organic layers were washed with brine (250 mL), dried over sodium sulfate, filtered and concentrated in vacuo. The crude mixture was purified by flash-chromatography on a silica gel cartridge (120 g Gold), using a gradient of 0 to 40% of EtOAc in heptanes to afford after evaporation methyl 2-(tert-butoxycarbonylamino)-3-(1-methylcyclobutyl)prop-2-enoate (8.6 g, 82%) as a white solid. ESI-MS m/z calc. 269.1627, found 214.2 (M-55)⁺; Retention time: 1.84 minutes, LC method X. E32460-10266 OmegaChem.

### Step 4: Methyl (2R)-2-(tert-butoxycarbonylamino)-3-(1-methylcyclobutyl)propanoate

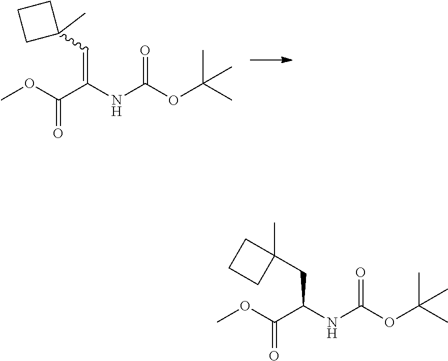 [SMILES: COC(=O)C(=CC1(C)CCC1)NC(=O)OC(C)(C)C ; COC(=O)[C@@H](CC1(C)CCC1)NC(=O)OC(C)(C)C]

Methyl 2-(tert-butoxycarbonylamino)-3-(1-methylcyclobutyl)prop-2-enoate (18 g, 65.962 mmol) was dissolved in ethanol (180 mL) and dioxane (90 mL). Nitrogen was passed through for 15 minutes and then 1,2-bis[(2R,5R)-2,5-diethylphospholano]benzene(1,5-cyclooctadiene)rhodium(I) trifluoromethanesulfonate (2.5 g, 3.4596 mmol) was added. Nitrogen was passed through for 5 minutes and then the mixture was hydrogenated under 65 psi hydrogen pressure and at room temperature for 4 hours. The mixture was concentrated in vacuo to dryness and a solution of EtOAc and heptanes (1:1, 200 mL) was then added to the mixture. The crude solution was filtered on a silica pad and the pad was rinsed with a solution of EtOAc and heptanes (1:1, 400 mL) to afford after evaporation methyl (2R)-2-(tert-butoxycarbonylamino)-3-(1-methylcyclobutyl)propanoate (17.5 g, 93%) as a yellow oil. ¹H NMR (400 MHz, CDCl₃) δ 4.91-4.77 (m, 1H), 4.39-4.24 (m, 1H), 3.72 (s, 3H), 2.01-1.76 (m, 5H), 1.75-1.64 (m, 3H), 1.45 (s, 9H), 1.23 (s, 3H). ESI-MS m/z calc. 271.1784, found 294.2 (M+23)⁺; Retention time: 1.9 minutes; LC method X.

### Step 5: tert-Butyl N-[(1R)-1-(hydroxymethyl)-2-(1-methylcyclobutyl)ethyl]carbamate

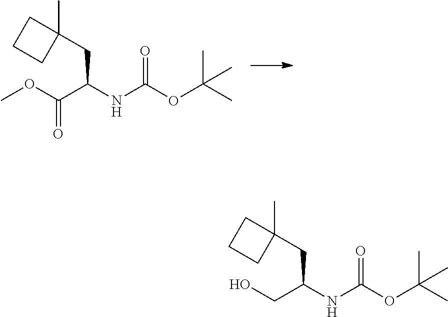 [SMILES: COC(=O)[C@@H](CC1(C)CCC1)NC(=O)OC(C)(C)C ; CC1(C[C@H](CO)NC(=O)OC(C)(C)C)CCC1]

A solution of methyl (2R)-2-(tert-butoxycarbonylamino)-3-(1-methylcyclobutyl)propanoate (17.5 g, 61.267 mmol) in THF (40 mL) at 0° C. was added to a suspension of LAH (3.5 g, 92.216 mmol) in THF (160 mL). The mixture was stirred at 0° C. for 15 minutes and then at room temperature for 2 hours. The mixture was then cooled down to 0° C. and water (3.5 mL) was added followed by an aqueous solution of NaOH (15%, 3.5 mL) and then by water (10.5 mL). The mixture was stirred at room temperature for 30 minutes and then magnesium sulfate (2 g) was added. The mixture was filtered on Celite and the filter cake was washed with EtOAc (100 mL). The filtrate was then concentrated in vacuo to afford the crude tert-butyl N-[(1R)-1-(hydroxymethyl)-2-(1-methylcyclobutyl)ethyl]carbamate (15.3 g, 97%) as a colorless oil. ¹H NMR (400 MHz, CDCl₃) δ 4.51 (br. s, 1H), 3.72 (br. s, 1H), 3.65-3.56 (m, 1H), 3.51-3.41 (m, 1H), 2.47 (br. s, 1H), 2.00-1.90 (m, 1H), 1.89-1.76 (m, 3H), 1.74-1.65 (m, 2H), 1.61-1.49 (m, 2H), 1.44 (s, 9H), 1.19 (s, 3H). ESI-MS m/z calc. 243.1834, found 188.2 (M-55)⁺; Retention time: 1.74 minutes; LC method X.

### Step 6: (2R)-2-Amino-3-(1-methylcyclobutyl)propan-1-ol

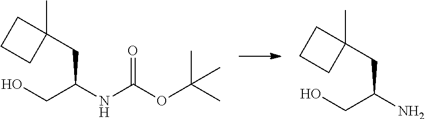

To a solution of tert-butyl N-[(1R)-1-(hydroxymethyl)-2-(1-methylcyclobutyl)ethyl]carbamate (15.3 g, 59.731 mmol) in dry DCM (150 mL) was added HCl (in dioxane) (150 mL of 4 M, 600.00 mmol) at room temperature. After 18 hours, the solvent was removed in vacuo to afford after co-evaporation with MeCN (2×100 mL) (2R)-2-amino-3-(1-methylcyclobutyl)propan-1-ol (hydrochloride salt) (11 g, 97%) as a white solid. ¹H NMR (400 MHz, DMSO-d₆) δ 7.93 (br. s, 3H), 5.33 (br. s, 1H), 3.56 (dd, J 11.5, 3.4 Hz, 1H), 3.39-3.31 (m, 1H, overlapped with water), 3.04 (br. s, 1H), 1.95-1.79 (m, 3H), 1.78-1.56 (m, 5H), 1.12 (s, 3H). Several batches from 3 different reactions of (2R)-2-amino-3-(1-methylcyclobutyl)propan-1-ol (hydrochloride salt) (1.25 g, 6.6087 mmol, 1.28 g, 6.7673 mmol, and 11 g, 58.156 mmol) were combined in water (75 mL). The resulting mixture was then lyophilized to afford (2R)-2-amino-3-(1-methylcyclobutyl)propan-1-ol (hydrochloride salt) (12.8 g, 95%) as a white solid. ¹H NMR (400 MHz, DMSO-d₆) δ 7.97 (br. s, 3H), 5.33 (br. s, 1H), 3.60-3.52 (m, 1H), 3.39-3.30 (m, 1H, overlapped with water), 3.04 (br. s, 1H), 1.94-1.79 (m, 3H), 1.78-1.57 (m, 5H), 1.12 (s, 3H). ESI-MS m/z calc. 143.13101, found 144.4 (M+1)⁺; Retention time: 0.56 minutes; LC method X.

### Step 7: Benzyl N-[(1R)-1-(hydroxymethyl)-2-(1-methylcyclobutyl)ethyl]carbamate

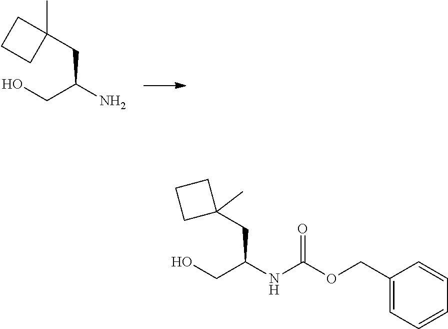

To a stirred suspension of (2R)-2-amino-3-(1-methylcyclobutyl)propan-1-ol (12.3 g, 81.584 mmol) in dry THF (250 mL) at 0° C. were added triethylamine (25.410 g, 35 mL, 251.11 mmol) followed by N-(benzyloxycarbonyloxy)succinimide (24.5 g, 98.307 mmol). The reaction was stirred at 0° C. for 15 minutes and then at room temperature for 4 hours. Water (250 mL) and EtOAc (250 mL) were added, and the mixture was extracted with EtOAc (3×250 mL). The combined organic layers were washed with water (3×250 mL) and brine (250 mL), dried over sodium sulfate, filtered, and concentrated in vacuo. The crude mixture was purified twice by flash-chromatography on a 330 g silica gel cartridge, eluting with a gradient of 0 to 100% of EtOAc in heptanes and then by reverse phase chromatography on a 275 g C₁₈ GOLD cartridge, eluting with a gradient of 40 to 100% of MeOH in acidic water (0.1% v/v of formic acid in water). The fractions containing the desired product were combined and the organic solvent was evaporated. EtOAc (500 mL) was then added, and the mixture was extracted with EtOAc (3×500 mL). The combined organic layers were washed with brine (1×500 mL), dried over sodium sulfate, filtered, and concentrated in vacuo. The product was then separated by SFC (Column Lux 5 m, Cellulose 4, 250×21.2 mm, 21.5 mg/injection, concentration 53.8 mg/mL, Injected volume 400 μL, Column T=40° C., Flow rate 75 mL/min, 20% MeOH). The fractions containing the desired product were combined and the solvent was evaporated to afford benzyl N-[(1R)-1-(hydroxymethyl)-2-(1-methylcyclobutyl)ethyl]carbamate (13.5 g, 58%) as a yellow oil. ¹H NMR (400 MHz, CDCl₃) δ 7.40-7.29 (m, 5H), 5.10 (s, 2H), 4.81 (br. s, 1H), 3.87-3.75 (m, 1H), 3.70-3.60 (m, 1H), 3.54-3.46 (m, 1H), 2.23 (br. s, 1H), 2.01-1.90 (m, 1H), 1.90-1.75 (m, 3H), 1.74-1.65 (m, 2H), 1.64-1.52 (m, 2H), 1.19 (s, 3H). ESI-MS m/z calc. 277.1678, found 278.2 (M+1)⁺; Retention time: 1.74 minutes; LC method X. The fractions containing the other enantiomer were combined and the solvent was concentrated in vacuo. The product was purified twice by reverse phase chromatography on a 80 g C₁₈ GOLD cartridge, eluting with a gradient of MeOH to 50% of 100 in acidic water (0.1% v/v of formic acid in water) and then on a 80 g C₁₈ GOLD cartridge, eluting with a gradient of MeCN to 50% of 100 in acidic water (0.1% v/v of formic acid in water). The fractions containing the desired product were combined and the organic solvent was evaporated. EtOAc (50 mL) was then added, and the mixture was extracted with EtOAc (3×50 mL). The combined organic layers were washed with brine (1×50 mL), dried over sodium sulfate, filtered and concentrated in vacuo to afford benzyl N-[(1S)-1-(hydroxymethyl)-2-(1-methylcyclobutyl)ethyl]carbamate (525 mg, 2%) as a yellow oil. ¹H NMR (400 MHz, CDCl₃) δ 7.38-7.31 (m, 5H), 5.11 (s, 2H), 4.80 (br. s, 1H), 3.86-3.76 (m, 1H), 3.71-3.62 (m, 1H), 3.55-3.47 (m, 1H), 2.00-1.90 (m, 1H), 1.88-1.76 (m, 3H), 1.74-1.65 (m, 2H), 1.64-1.52 (m, 2H), 1.19 (s, 3H), 1 missing proton (labile proton). ESI-MS m/z calc. 277.1678, found 278.2 (M+1)⁺; Retention time: 1.75 minutes; LC method X.

### Step 8: (2R)-2-Amino-3-(1-methylcyclobutyl)propan-1-ol

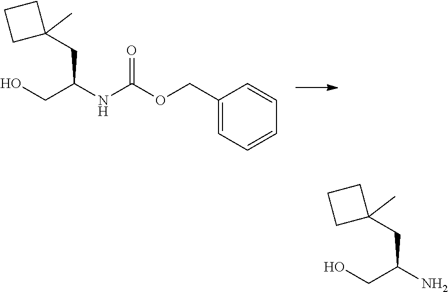

To a degassed solution of benzyl N-[(1R)-1-(hydroxymethyl)-2-(1-methylcyclobutyl)ethyl]carbamate (13.5 g, 47.165 mmol) in methanol (250 mL) was added 10% Palladium on carbon 50% wet (5.2 g, 2.4431 mmol). After purging with nitrogen for 5 minutes, hydrogen was bubbled into the solution for 5 minutes after which the mixture was stirred at room temperature under a hydrogen atmosphere (1 atm.) for 6 hours. The mixture was filtered through a pad of Celite® and the pad was rinsed with methanol (100 mL). The filtrate was concentrated in vacuo and then acidified by adding hydrogen chloride solution (in methanol) (50 mL of 3 M, 150.00 mmol) to the product. The mixture was stirred 5 minutes at room temperature and then concentrated in vacuo to afford after lyophilization (2R)-2-amino-3-(1-methylcyclobutyl)propan-1-ol (hydrochloride salt) (7.68 g, 86%) as a white solid. ¹H NMR (400 MHz, DMSO-d₆) δ 7.28 (br. s, 3H), 5.23 (br. s, 1H), 3.57-3.47 (m, 1H), 3.34-3.25 (m, 1H), 3.05-2.95 (m, 1H), 1.95-1.70 (m, 4H), 1.70-1.58 (m, 3H), 1.58-1.50 (m, 1H), 1.11 (s, 3H). ESI-MS m/z calc. ESI-MS m/z calc. 143.13101, found 144.4 (M+1)⁺; Retention time: 0.64 minutes; LC method X.

### Step 9: 3-[[4-[(2R)-2-Amino-3-(1-methylcyclobutyl)propoxy]-6-(2,6-dimethylphenyl)pyrimidin-2-yl]sulfamoyl]benzoic acid

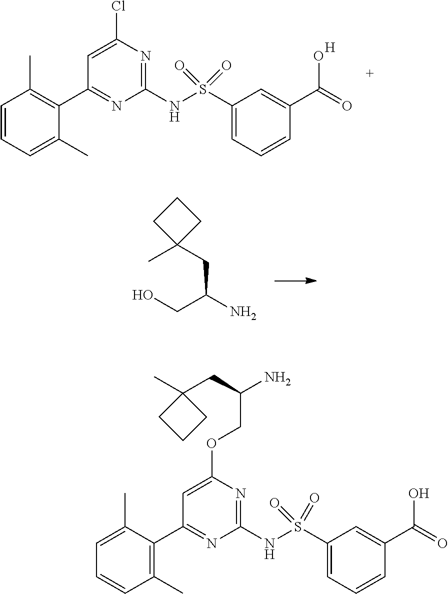

A solution of (2R)-2-amino-3-(1-methylcyclobutyl)propan-1-ol (7.65 g, 50.741 mmol) in anhydrous N,N-dimethylformamide (40 mL) was added to a solution of 3-[[4-chloro-6-(2,6-dimethylphenyl)pyrimidin-2-yl]sulfamoyl]benzoic acid (23 g, 55.042 mmol) in 2-methyltetrahydrofuran (200 mL). The mixture was cooled down to 10-15° C. and then sodium tert-butoxide (30 g, 312.16 mmol) was added. The reaction was stirred at 10-15° C. for 2 hours, then cooled down to 0° C. and quenched by the addition of an aqueous solution of 1N HCl (300 mL). The biphasic mixture was stirred for 30 minutes. The layers were then separated, and the aqueous layer was extracted with 2-methyltetrahydrofuran (5×500 mL). The combined organic layers were washed with water (3×500 mL) and brine (1×500 mL), dried over magnesium sulfate, filtered and concentrated in vacuo. The crude mixture was purified by reverse phase chromatography on a 275 g C₁₈ GOLD cartridge, eluting with a gradient of 20 to 100% of MeOH in acidic water (0.1% of hydrochloric acid in water) to afford after evaporation 3-[[4-[(2R)-2-amino-3-(1-methylcyclobutyl)propoxy]-6-(2,6-dimethylphenyl)pyrimidin-2-yl]sulfamoyl]benzoic acid (hydrochloride salt) (24.25 g, 78%) as a white solid. ¹H NMR (400 MHz, DMSO-d₆) δ 8.48-8.35 (m, 4H), 8.13 (t, J 9.3 Hz, 2H), 7.71 (t, J 7.7 Hz, 1H), 7.25 (t, J 7.6 Hz, 1H), 7.12 (d, J 7.6 Hz, 2H), 6.33 (s, 1H), 4.32 (dd, J 11.6, 2.6 Hz, 1H), 4.06 (dd, J 11.7, 6.1 Hz, 1H), 3.47 (br. s, 1H), 2.00 (s, 6H), 1.93-1.83 (m, 2H), 1.82-1.62 (m, 5H), 1.58-1.47 (m, 1H), 1.16 (s, 3H). 2H missing, labile protons. ESI-MS m/z calc. 524.20935, found 525.3 (M+1)⁺; Retention time: 2.49 minutes; LC method Y.

### Step 10: 3-[[4-(2,6-Dimethylphenyl)-6-[(2R)-2-[(5-isopropoxypyrimidin-2-yl)methylamino]-3-(1-methylcyclobutyl)propoxy]pyrimidin-2-yl]sulfamoyl]benzoic acid

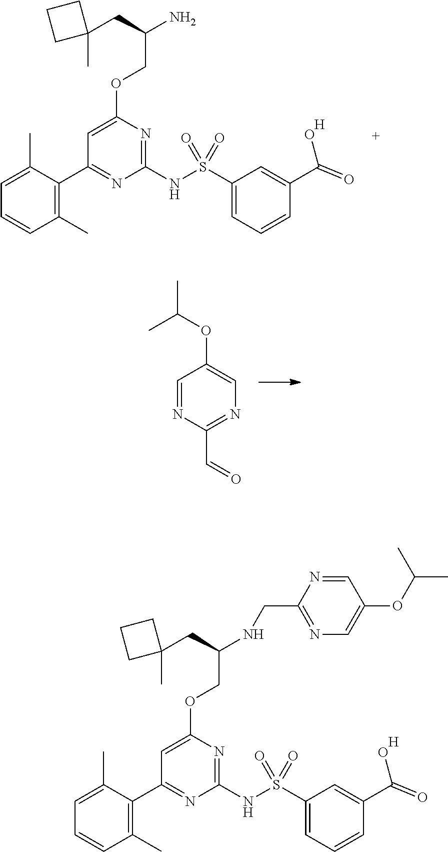

A 4 mL vial was charged under nitrogen with 3-[[4-[(2R)-2-amino-3-(1-methylcyclobutyl)propoxy]-6-(2,6-dimethylphenyl)pyrimidin-2-yl]sulfamoyl]benzoic acid (hydrochloride salt) (107 mg, 0.1907 mmol), 5-isopropoxypyrimidine-2-carbaldehyde (36 mg, 0.2166 mmol), anhydrous DCM (0.85 mL) and acetic acid (15 μL, 0.2638 mmol). The mixture was cooled down in an ice bath. DIEA (73 μL, 0.4191 mmol) was added (partial dissolution of solids) followed quickly by sodium triacetoxyborohydride (228 mg, 1.076 mmol). The reaction was vigorously stirred at 0° C. for one hour. An additional amount of 5-isopropoxypyrimidine-2-carbaldehyde (10 mg, 0.06018 mmol) was added and the reaction was stirred for an additional 1.5 h. Aqueous 1N HCl, brine and EtOAc were added, and the two phases were separated. The aqueous phase was further extracted with EtOAc (3×). The combined organic phases were dried over sodium sulfate and evaporated. The residue was purified by flash chromatography on silica gel (12 g column) using a gradient of methanol (0 to 15% over 20 min) in dichloromethane. After evaporation, the residue was treated several times with additions/evaporations of DCM/hexanes to remove any residual methanol. 3-[[4-(2,6-dimethylphenyl)-6-[(2R)-2-[(5-isopropoxypyrimidin-2-yl)methylamino]-3-(1-methylcyclobutyl)propoxy]pyrimidin-2-yl]sulfamoyl]benzoic acid (hydrochloride salt) (46 mg, 34%) was isolated as a white solid. ESI-MS m/z calc. 674.28864, found 675.68 (M+1)⁺; Retention time: 1.44 minutes; LC method A.

### Step 11: (11R)-6-(2,6-Dimethylphenyl)-12-[(5-isopropoxypyrimidin-2-yl)methyl]-11-[(1-methylcyclobutyl)methyl]-2,2-dioxo-9-oxa-2λ6-thia-3,5,12,19-tetrazatricyclo[12.3.1.14,8]nonadeca-1(18),4(19),5,7,14,16-hexaen-13-one (Compound 1317)

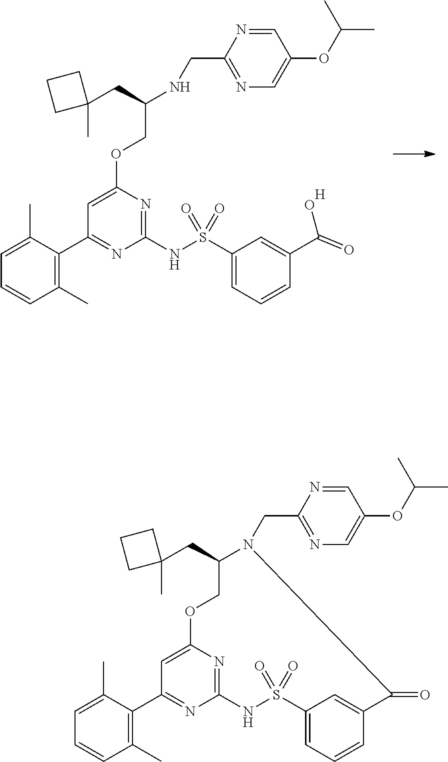

3-[[4-(2,6-Dimethylphenyl)-6-[(2R)-2-[(5-isopropoxypyrimidin-2-yl)methylamino]-3-(1-methylcyclobutyl)propoxy]pyrimidin-2-yl]sulfamoyl]benzoic acid (hydrochloride salt) (46 mg, 0.06467 mmol) was combined in a flask under nitrogen with CDMT (24 mg, 0.1367 mmol) and DMF (5 mL). The solution was stirred at 0° C. 4-Methyl-morpholine (40 μL, 0.3638 mmol) was added and the mixture was stirred in the cooling bath that was allowed to warm to room temperature for 22 h. The reaction was filtered and purified by reverse phase HPLC (1-99% acetonitrile/5 mM aqueous HCl over 15 min) to give (11R)-6-(2,6-dimethylphenyl)-12-[(5-isopropoxypyrimidin-2-yl)methyl]-11-[(1-methylcyclobutyl)methyl]-2,2-dioxo-9-oxa-2λ⁶-thia-3,5,12,19-tetrazatricyclo[12.3.1.14,8] nonadeca-1(18),4(19),5,7,14,16-hexaen-13-one (28 mg, 64%) as a white solid. ¹H NMR (400 MHz, DMSO-d₆) δ 13.37-11.64 (broad m, 1H), 8.65 (s, 1H), 8.52 (s, 2H), 7.94 (s, 1H), 7.75-7.46 (broad m, 2H), 7.33-7.20 (m, 1H), 7.12 (d, J 7.8 Hz, 2H), 6.43 (br s, 1H), 5.42-5.29 (m, 1H), 4.88 (d, J 16.5 Hz, 1H), 4.80 (h, J 6.0 Hz, 1H), 4.69 (d, J 16.6 Hz, 1H), 4.20 (t, J 11.3 Hz, 1H), 4.09-3.96 (m, 1H), 2.24-1.86 (m, 7H), 1.81-1.67 (m, 2H), 1.65-1.57 (m, 1H), 1.51 (d, J 14.9 Hz, 1H), 1.47-1.39 (m, 1H), 1.34-1.26 (m, 8H), 0.60 (s, 3H). ESI-MS m/z calc. 656.2781, found 657.71 (M+1)⁺; Retention time: 1.96 minutes; LC method A.

### Example 159: Preparation of Compound 1318

### Step 1: 2-Bromooxazole-4-carbaldehyde

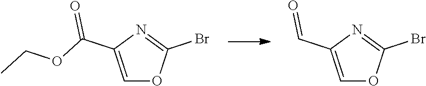

A flame-dried flask under nitrogen atmosphere was charged with ethyl 2-bromooxazole-4-carboxylate (5 g, 22.828 mmol) and anhydrous tetrahydrofuran (100 mL). The solution was cooled down to −78° C. and a solution of DIBAL-H in toluene (90 mL of 1 M, 90.000 mmol) was added dropwise. The reaction was stirred at −78° C. for 5 hours then quenched by the addition of a saturated aqueous solution of sodium potassium tartrate (300 mL). The mixture was then allowed to reach room temperature then stirred at room temperature under nitrogen atmosphere for 2 days. The layers were separated, and the aqueous layer was extracted with dichloromethane (5×100 mL). The combined organic extracts were washed with water (250 mL), brine (250 mL), dried over sodium sulfate, filtered, and concentrated under reduced pressure to afford crude 2-bromooxazole-4-carbaldehyde (1.383 g, 33%) as an off-white powder which was used in the next step without further purification. ¹H NMR (400 MHz, CDCl₃) δ 9.89 (s, 1H), 8.29 (s, 1H). ESI-MS m/z calc. 174.9269, found 175.9 (M+1)⁺; Retention time: 1.66 minutes; LC method 1D.

### Step 2: 3-[[4-[(2R)-2-[(2-Bromooxazol-4-yl)methylamino]-4,4-dimethyl-pentoxy]-6-(2,6-dimethylphenyl)pyrimidin-2-yl]sulfamoyl]benzoic acid

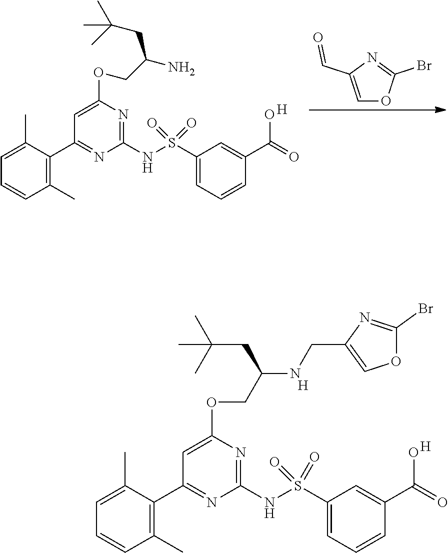

A flask containing 500 mg of 4A molecular sieves was flame-dried under vacuum then cooled down with a nitrogen stream. Then 3-[[4-[(2R)-2-amino-4,4-dimethyl-pentoxy]-6-(2,6-dimethylphenyl)pyrimidin-2-yl]sulfamoyl]benzoic acid (hydrochloride salt) (4.35 g, 6.6436 mmol) and anhydrous dichloromethane (100 mL) were added, followed by a solution of 2-bromooxazole-4-carbaldehyde (1.383 g, 7.5843 mmol) in anhydrous dichloromethane (20 mL) and acetic acid (42.240 mg, 40 μL, 0.7034 mmol). The reaction was stirred at 10-15° C. for 1 h then sodium triacetoxyborohydride (4.85 g, 22.884 mmol) was added. The reaction was stirred at 10-15° C. for 1.5 h, then quenched by the addition of a cold aqueous solution of 1N hydrochloric acid (500 mL). The layers were separated, and the aqueous layer was extracted with ethyl acetate (4×150 mL). The combined organic layers were washed with water (200 mL), brine (250 mL), dried over magnesium sulfate, filtered and concentrated under reduced pressure to afford 3-[[4-[(2R)-2-[(2-bromooxazol-4-yl)methylamino]-4,4-dimethyl-pentoxy]-6-(2,6-dimethylphenyl)pyrimidin-2-yl]sulfamoyl]benzoic acid (hydrochloride salt) (5.62 g, 58%) as a white foam which was directly used in the next step without further purification. ESI-MS m/z calc. 671.1413, found 672.0 (M+1)⁺; Retention time: 1.45 minutes; LC method X.

### Step 3: (11R)-12-[(2-Bromooxazol-4-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

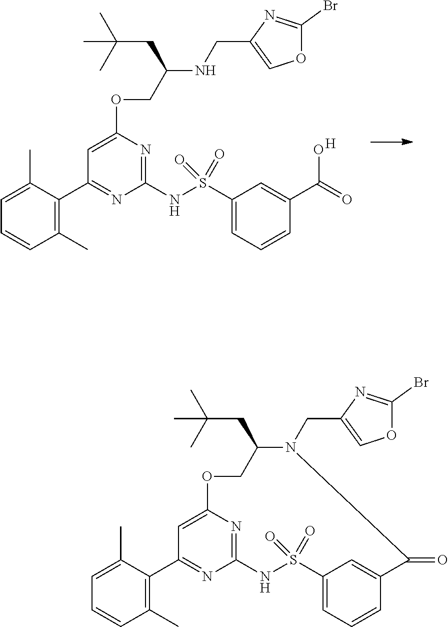

To a stirred solution of crude 3-[[4-[(2R)-2-[(2-bromooxazol-4-yl)methylamino]-4,4-dimethyl-pentoxy]-6-(2,6-dimethylphenyl)pyrimidin-2-yl]sulfamoyl]benzoic acid (hydrochloride salt) (5.62 g, 4.3990 mmol) in anhydrous N,N-dimethylformamide (90 mL) were added 4-methylmorpholine (2.7600 g, 3 mL, 27.287 mmol) and 2-chloro-4,6-dimethoxy-1,3,5-triazine (1.5 g, 8.5435 mmol) at 0° C. The reaction was stirred for 15 minutes at 0° C. then allowed to reach room temperature and stirred overnight at room temperature. Volatiles were removed under reduced pressure and the yellow residue was purified by reverse phase chromatography on a C₁₈ using a 120 g column and eluting with a gradient of acetonitrile in acidic water (containing 0.1% v/v of formic acid, 5 to 100% in 20 CV). The volatiles were removed under reduced pressure and the aqueous layer was neutralized by the addition of a saturated aqueous solution of sodium bicarbonate (250 mL). The aqueous layer was then extracted with ethyl acetate (5×100 mL) and the combined organic layers were washed with brine (200 mL), dried over magnesium sulfate, filtered and concentrated under reduced pressure to afford an—off white foam which was freeze-dried to afford (11R)-12-[(2-bromooxazol-4-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (1.441 g, 48%) as a white powder. ¹H NMR (400 MHz, DMSO-d₆) δ 13.33-12.82 (m, 1H), 8.47 (br. s, 1H), 8.24 (s, 1H), 8.02-7.86 (m, 1H), 7.74-7.58 (m, 2H), 7.32-7.19 (m, 1H), 7.17-7.05 (m, 2H), 6.54-6.32 (m, 1H), 5.16 (dd, J 11.6, 4.5 Hz, 1H), 4.68-4.52 (m, 1H), 4.45-4.26 (m, 2H), 3.99-3.82 (m, 1H), 2.11-1.83 (m, 7H), 1.38 (d, J 14.4 Hz, 1H), 0.49 (s, 9H). ESI-MS m/z calc. 653.13074, found 654.1 (M+1)⁺; Retention time: 4.38 minutes; LC method Y.

### Step 4: (11R)-12-[[2-[Cyclobutyl(methyl)amino]oxazol-4-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1318)

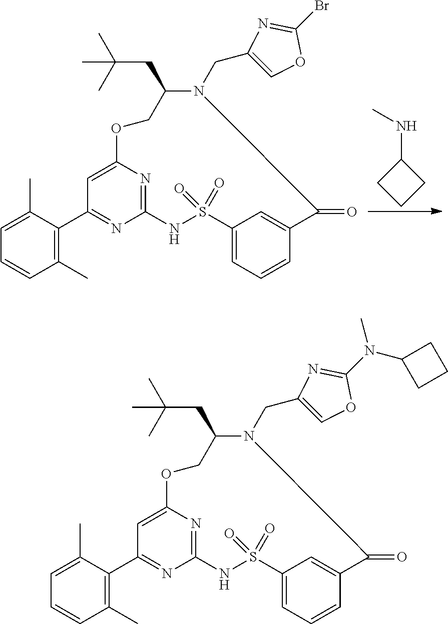

In a tube, to a solution of (11R)-12-[(2-bromooxazol-4-yl)methyl]-6-(2,6-dimethylphenyl)-11-(2,2-dimethylpropyl)-2,2-dioxo-9-oxa-2?⁶-thia-3,5,12,19-tetrazatricyclo[12.3.1.14,8]nonadeca-1(18),4(19),5,7,14,16-hexaen-13-one (60 mg, 0.0879 mmol) in dry tetrahydrofuran (0.5 mL) was added N-methylcyclobutanamine (hydrochloride salt) (32 mg, 0.2631 mmol) followed by sodium tert-butoxide (25 mg, 0.2601 mmol). Nitrogen was bubbled for 5 minutes and then dichloro[1,3-bis(2,6-di-3-pentylphenyl)imidazol-2-ylidene](3-chloropyridyl)palladium(II) (Pd-PEPPSITM-IPent catalyst, 5 mg, 0.0063 mmol) was added. The tube was sealed, and the reaction was stirred 48 hours at 60° C. The crude mixture was then cooled down to room temperature and was concentrated in vacuo. The crude product was purified twice by reverse phase chromatography on a 30 g C₁₈ GOLD cartridge, eluting with a gradient of 5 to 100% of MeCN in basic water (ammonium bicarbonate/ammonium hydroxide buffer pH=10) and then on a 30 g C₁₈ GOLD cartridge, eluting with a gradient of 5 to 100% of MeCN in acidic water (0.1% v/v of formic acid in water) to afford after co-evaporation with water (1×5 mL) and lyophilization (11R)-12-[[2-[cyclobutyl(methyl)amino]oxazol-4-yl]methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (36.5 mg, 61%) as a white solid. ¹H NMR (400 MHz, DMSO-d₆) δ 13.03 (br. s, 1H), 8.51 (br. s, 1H), 7.92 (br. s, 1H), 7.76-7.56 (m, 2H), 7.43 (s, 1H), 7.30-7.21 (m, 1H), 7.19-7.05 (m, 2H), 6.40 (br. s, 1H), 5.24 (dd, J 10.8, 4.2 Hz, 1H), 4.53 (d, J 15.4 Hz, 1H), 4.44-4.27 (m, 2H), 4.17 (d, J 15.7 Hz, 1H), 3.93-3.81 (m, 1H), 2.94 (s, 3H), 2.27-2.06 (m, 6H), 2.04-1.81 (m, 5H), 1.69-1.58 (m, 2H), 1.37 (d, J 15.2 Hz, 1H), 0.52 (s, 9H). ESI-MS m/z calc. 658.29376, found 659.3 (M+1)⁺; Retention time: 4.65 minutes; LC method Y.

### Example 160: Preparation of Compound 1319

### Step 1: 3-[[4-[(2R)-2-Amino-5,5-dimethyl-hexoxy]-6-(2,6-dimethylphenyl)pyrimidin-2-yl]sulfamoyl]benzoic acid

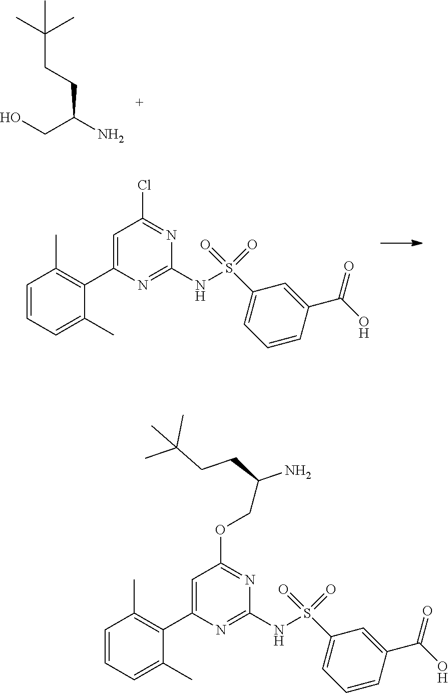

A solution of (2R)-2-amino-5,5-dimethyl-hexan-1-ol (hydrochloride salt) (4.495 g, 23.501 mmol) in anhydrous DMF (23 mL) was added to a solution of 3-[[4-chloro-6-(2,6-dimethylphenyl)pyrimidin-2-yl]sulfamoyl]benzoic acid (13.5 g, 32.384 mmol) in Me-THF (117 mL). The mixture was cooled down to 10-15° C. (inner temperature) and then sodium tert-butoxide (17.4 g, 181.05 mmol) was added. The reaction was stirred at 10-15° C. for 2 hours, then cooled down to 0° C. and quenched by the addition of an aqueous solution of 1N HCl (180 mL) at 0° C. The biphasic mixture was stirred for 30 minutes. The layers were then separated, and the aqueous layer was extracted with 2-methyltetrahydrofuran (5×500 mL). The combined organic layers were washed with water (3×500 mL) and brine (1×500 mL), dried over magnesium sulfate, filtered and concentrated in vacuo. The residue was dissolved in MeOH (75 mL) and precipitated in EtOAc (800 mL). the solid was filtered over a glass frit (por.4) and the residue was collected using MeOH to dissolve it. The crude mixture was purified by reverse phase chromatography on a 275 g C₁₈ GOLD cartridge, eluting with a gradient of 0 to 80% of CH₃CN in acidic water (0.1% of hydrochloric acid in water) to afford after evaporation 3-[[4-[(2R)-2-amino-5,5-dimethyl-hexoxy]-6-(2,6-dimethylphenyl)pyrimidin-2-yl]sulfamoyl]benzoic acid (hydrochloride salt) (10.2 g, 70%) as a white solid. ¹H NMR (400 MHz, DMSO-d₆) δ 8.45 (t, J 1.7 Hz, 1H), 8.38 (br. s, 3H), 8.18-8.09 (m, 2H), 7.70 (t, J 7.8 Hz, 1H), 7.29-7.21 (m, 1H), 7.12 (d, J 7.8 Hz, 2H), 6.31 (s, 1H), 4.43-4.36 (m, 1H), 4.35-4.27 (m, 1H), 3.45 (br. s, 1H), 1.99 (s, 6H), 1.70-1.49 (m, 2H), 1.31 (td, J 12.6, 4.9 Hz, 1H), 1.21-1.11 (m, 1H), 0.83 (s, 9H), 1H labile missing (from —COOH). ESI-MS m/z calc. 526.225, found 527.2 (M+1)⁺; Retention time: 2.58 minutes; LC method A.

### Step 2: 3-[[4-[(2R)-2-[[6-[Cyclobutyl(methyl)amino]pyrazin-2-yl]methylamino]-5,5-dimethyl-hexoxy]-6-(2,6-dimethylphenyl)pyrimidin-2-yl]sulfamoyl]benzoic acid

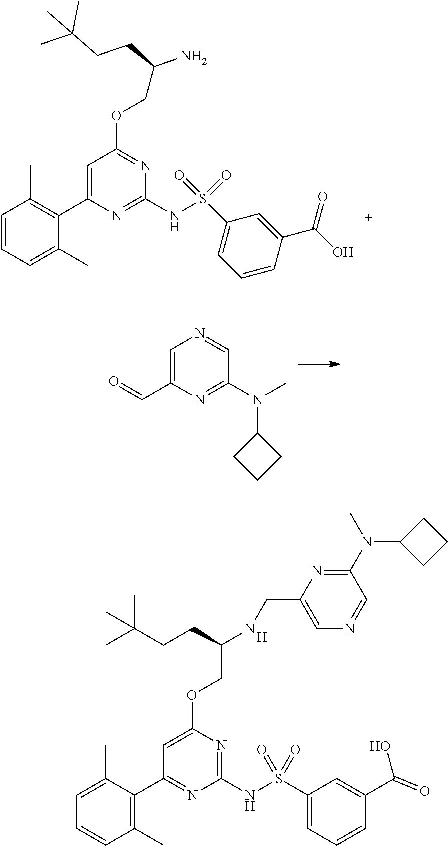

In a 4 mL vial, to a stirred mixture of 3-[[4-[(2R)-2-amino-5,5-dimethyl-hexoxy]-6-(2,6-dimethylphenyl)pyrimidin-2-yl]sulfamoyl]benzoic acid (hydrochloride salt) (75 mg, 0.1321 mmol) and 6-[cyclobutyl(methyl)amino]pyrazine-2-carbaldehyde (27 mg, 0.1348 mmol) in anhydrous dichloromethane (0.5 mL) was added glacial acetic acid (10 μL, 0.1758 mmol) and DIPEA (70 μL, 0.4019 mmol), in that order, at 0° C. under nitrogen. After 2-3 min, sodium triacetoxyborohydride (100 mg, 0.4718 mmol) was added to the yellow solution. The heterogeneous reaction was stirred at that temperature for 15 min. Then the reaction was quenched with aqueous 1M HCl (1 mL), MeOH (1 mL) and DMSO (1 mL) and purified by preparative reverse-phase HPLC [1-99% acetonitrile in water (containing 5 mM HCl) over 15 min] to furnish 3-[[4-[(2R)-2-[[6-[cyclobutyl(methyl)amino]pyrazin-2-yl]methylamino]-5,5-dimethyl-hexoxy]-6-(2,6-dimethylphenyl)pyrimidin-2-yl]sulfamoyl]benzoic acid (hydrochloride salt) (78 mg, 80%) as a yellow solid. ESI-MS m/z calc. 701.33594, found 702.3 (M+1)⁺; Retention time: 1.46 minutes; LC method A.

### Step 3: (11R)-12-[[6-[Cyclobutyl(methyl)amino]pyrazin-2-yl]methyl]-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1319)

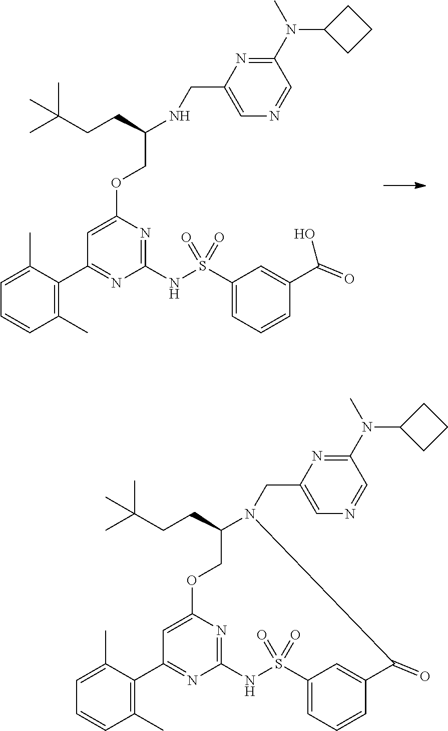 [SMILES: Cc1cccc(C)c1-c1cc(OC[C@@H](CCC(C)(C)C)NCc2cncc(N(C)C3CCC3)n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cncc(N(C)C3CCC3)n1)[C@H](CCC(C)(C)C)CO2]

To a stirred solution of 3-[[4-[(2R)-2-[[6-[cyclobutyl(methyl)amino]pyrazin-2-yl]methylamino]-5,5-dimethyl-hexoxy]-6-(2,6-dimethylphenyl)pyrimidin-2-yl]sulfamoyl]benzoic acid (hydrochloride salt) (78 mg, 0.1056 mmol) in anhydrous DMF (4 mL) was added 2-chloro-4,6-dimethoxy-1,3,5-triazine (50 mg, 0.2848 mmol) (CDMT), followed by addition of 4-methylmorpholine (80 μL, 0.7277 mmol) at 0-4° C. (ice-water bath) under nitrogen. The yellow reaction was allowed to stir at that temperature for 5 min, then allowed to stir at room temperature for 13 h (overnight). After removing most of the volatiles under reduced pressure, the residue was purified by preparative reverse-phase HPLC (1-99% acetonitrile in water over 15 min, 5 mM HCl as modifier) to furnish (11R)-12-[[6-[cyclobutyl(methyl)amino]pyrazin-2-yl]methyl]-11-(3,3-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (32 mg, 44%) as a yellow solid. ¹H NMR (400 MHz, CDCl₃) δ 8.76 (t, J 1.8 Hz, 1H), 8.07 (d, J 7.8 Hz, 1H), 7.90 (s, 1H), 7.88-7.81 (m, 2H), 7.68 (t, J 7.8 Hz, 1H), 7.22 (t, J 7.6 Hz, 1H), 7.06 (d, J 7.6 Hz, 2H), 6.24 (s, 1H), 5.42 (d, J 7.0 Hz, 1H), 5.15 (d, J 15.7 Hz, 1H), 4.58-4.45 (m, 1H), 4.14-4.01 (m, 3H), 3.19 (s, 3H), 2.40-2.28 (m, 2H), 2.28-2.18 (m, 2H), 2.04 (s, 6H), 1.82-1.71 (m, 2H), 1.68-1.56 (m, 2H), 1.20-1.07 (m, 1H), 0.81 (s, 9H), 0.80-0.74 (m, 1H). ESI-MS m/z calc. 683.3254, found 684.8 (M+1)⁺; Retention time: 2.0 minutes; LC method A.

### Example 161: Preparation of Compound 1320 and Compound 1321

### Step 1: Methyl 2-(tert-butoxycarbonylamino)-2-spiro[3.3]heptan-2-ylidene-acetate

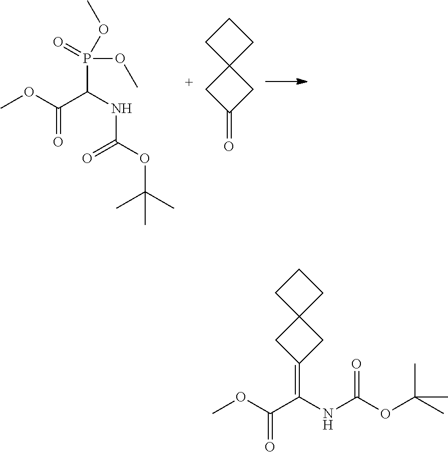 [SMILES: COC(=O)C(NC(=O)OC(C)(C)C)P(=O)(OC)OC ; O=C1CC2(CCC2)C1 ; COC(=O)C(NC(=O)OC(C)(C)C)=C1CC2(CCC2)C1]

To a solution of methyl 2-(tert-butoxycarbonylamino)-2-dimethoxyphosphoryl-acetate (17.55 g, 59.043 mmol) in ethyl acetate (175 mL) was added 1,1,3,3-tetramethylguanidine (9.1800 g, 10 mL, 79.703 mmol). The reaction mixture was stirred at room temperature for 35 minutes, followed by the addition of a solution of spiro[3.3]heptan-2-one (8.47 g, 76.892 mmol) in ethyl acetate (63 mL). The reaction mixture was stirred at room temperature for 6 days then quenched with the addition of a 1N aqueous solution of HCl (250 mL). The phases were separated, and the aqueous layer was extracted with ethyl acetate (3×200 mL). The combined organic layers were washed with a saturated solution of potassium bicarbonate (200 mL) and brine (200 mL) then dried over anhydrous sodium sulfate, filtered, and concentrated under reduced pressure. The crude residue was purified by silica gel on a 330-g column, eluting from 0% to 10% ethyl acetate in heptanes, to afford methyl 2-(tert-butoxycarbonylamino)-2-spiro[3.3]heptan-2-ylidene-acetate (9.38 g, 52%) as a pale-yellow oil. ESI-MS m/z calc. 281.1627, found 226.2 (M-56)⁺; Retention time: 1.92 minutes. LC method X.

### Step 2: Methyl 2-(tert-butoxycarbonylamino)-2-spiro[3.3]heptan-2-yl-acetate

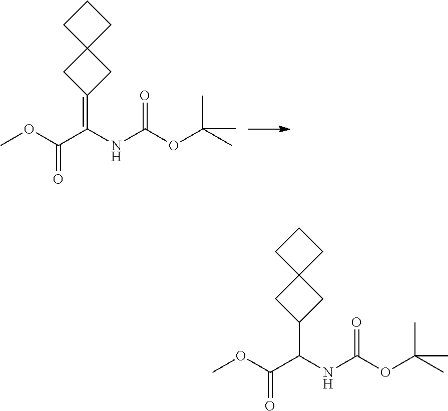 [SMILES: COC(=O)C(NC(=O)OC(C)(C)C)=C1CC2(CCC2)C1 ; COC(=O)C(NC(=O)OC(C)(C)C)C1CC2(CCC2)C1]

To a solution of methyl 2-(tert-butoxycarbonylamino)-2-spiro[3.3]heptan-2-ylidene-acetate (9.38 g, 30.973 mmol) in MeOH (185 mL) was added palladium on carbon (10%, 50% wet) (3.3 g, 5% w/w, 1.5505 mmol). The reaction was bubbled with nitrogen for 5 minutes then bubbled with hydrogen for 5 min. The reaction mixture was stirred at room temperature under 1 atm. of hydrogen for 2 hours. The reaction mixture was filtered on a Celite pad and the pad was rinsed with MeOH (10 mL). The crude residue was purified by silica gel on a 330-g column, eluting from 0% to 10% ethyl acetate in heptanes, to afford methyl 2-(tert-butoxycarbonylamino)-2-spiro[3.3]heptan-2-yl-acetate (7.04 g, 76%) as a colorless oil. ¹H NMR (400 MHz, CDCl₃) δ 4.91 (d, J 7.9 Hz, 1H), 4.19 (t, J 8.2 Hz, 1H), 3.71 (s, 3H), 2.47-2.33 (m, 1H), 2.07-1.94 (m, 4H), 1.91-1.84 (m, 3H), 1.82-1.73 (m, 3H), 1.44 (s, 9H). ESI-MS m/z calc. 283.1784, found 228.2 (M-56)⁺; Retention time: 1.97 minutes, LC method X.

### Step 3: tert-Butyl N-(2-hydroxy-1-spiro[3.3]heptan-2-yl-ethyl)carbamate

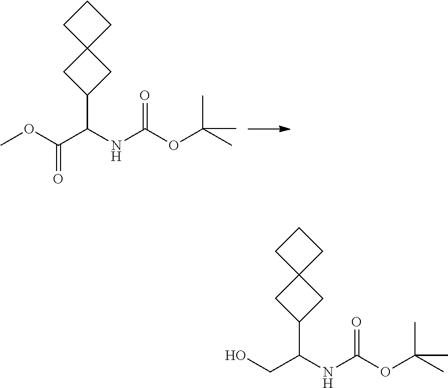 [SMILES: COC(=O)C(NC(=O)OC(C)(C)C)C1CC2(CCC2)C1 ; CC(C)(C)OC(=O)NC(CO)C1CC2(CCC2)C1]

To a solution of methyl 2-(tert-butoxycarbonylamino)-2-spiro[3.3]heptan-2-yl-acetate (7.04 g, 24.844 mmol) in THF (50 mL) was added a LiBH₄ solution in THF (30 mL of 2 M, 60.000 mmol). The reaction mixture was stirred at room temperature for 4 hours then poured slowly over a saturated aqueous solution of ammonium chloride (100 mL) at 0° C. The product was extracted with EtOAc (3×50 mL). The combined organic layers were washed with brine (100 mL), dried with sodium sulfate, filtered and concentrated under reduced pressure to afford crude tert-butyl N-(2-hydroxy-1-spiro[3.3]heptan-2-yl-ethyl)carbamate (6.58 g, 99%) as a white solid. ¹H NMR (400 MHz, CDCl₃) δ 4.52 (br. s., 1H), 3.69-3.58 (m, 1H), 3.57-3.48 (m, 1H), 3.47-3.39 (m, 1H), 2.57 (br. s., 1H), 2.26-2.12 (m, 1H), 2.11-1.94 (m, 4H), 1.93-1.86 (m, 2H), 1.85-1.69 (m, 4H), 1.45 (s, 9H). ESI-MS m/z calc. 255.1834, found 200.2 (M-56)⁺; Retention time: 1.81 minutes, LC method X.

### Step 4: 2-Amino-2-spiro[3.3]heptan-2-yl-ethanol

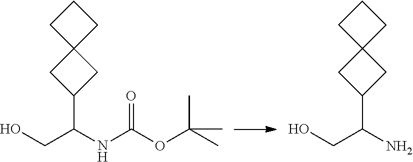 [SMILES: CC(C)(C)OC(=O)NC(CO)C1CC2(CCC2)C1 ; NC(CO)C1CC2(CCC2)C1]

To a solution of tert-butyl N-(2-hydroxy-1-spiro[3.3]heptan-2-yl-ethyl)carbamate (6.58 g, 25.768 mmol) in 1,4-dioxane (20 mL) was added a HCl solution in 1,4-dioxane (45 mL of 4 M, 180.00 mmol). The reaction mixture was stirred at room temperature for 20 hours then a HCl solution in 1,4-dioxane (20 mL of 4 M, 80.000 mmol) was again added. The reaction was stirred 4 more hours and the reaction was concentrated. The resulting solid was diluted in acetonitrile (1 mL) and water (5 mL) and was lyophilized to afford 2-amino-2-spiro[3.3]heptan-2-yl-ethanol (hydrochloride salt) (4.55 g, 88%) as a white solid. ¹H NMR (400 MHz, DMSO-d₆) δ 7.83 (br. s., 3H), 5.20 (t, J 5.0 Hz, 1H), 3.56-3.45 (m, 1H), 3.37-3.26 (m, 1H), 2.99-2.87 (m, 1H), 2.36-2.19 (m, 1H), 2.09-1.91 (m, 4H), 1.90-1.66 (m, 6H). ESI-MS m/z calc. 155.131, found 156.2 (M+1)⁺; Retention time: 1.64 minutes, LC method Y.

### Step 5: 3-[[4-(2-Amino-2-spiro[3.3]heptan-2-yl-ethoxy)-6-(2,6-dimethylphenyl)pyrimidin-2-yl]sulfamoyl]benzoic acid

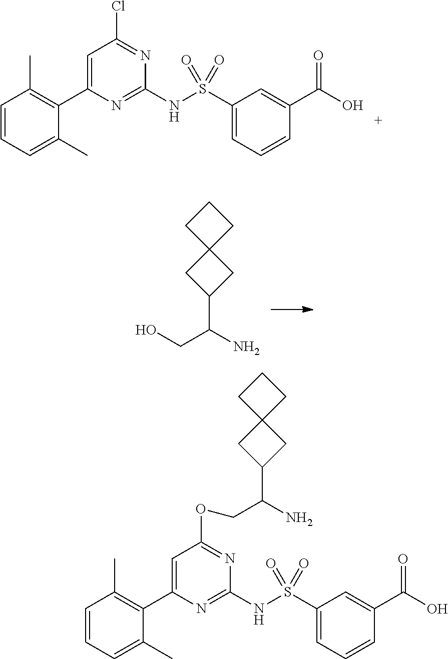

To a solution of 2-amino-2-spiro[3.3]heptan-2-yl-ethanol (hydrochloride salt) (0.998 g, 5.2061 mmol) and 3-[[4-chloro-6-(2,6-dimethylphenyl)pyrimidin-2-yl]sulfamoyl]benzoic acid (hydrochloride salt) (2.375 g, 5.2275 mmol) in THF (12 mL) was added sodium tert-butoxide (2.507 g, 26.086 mmol). The reaction was stirred at room temperature for 30 minutes then THE (10 mL) and 2-methyl THF (5 mL) were added. The reaction was stirred for another 30 minutes then an aqueous solution of HCl 1N (10 mL) was added. The reaction was diluted with 2-methyl THF (20 mL) and the phase were separated. The aqueous layers were extracted with 2-methyl THF (3×20 mL) and the combined organic layers were dried over anhydrous sodium sulfate, filtered and concentrated under reduced pressure. The solid was diluted in EtOAc (100 mL) and stirred for 30 minutes then filtered on Buchner funnel. The solid was again diluted in EtOAc (100 mL) and stirred for 1 hour then filtered and dried under reduced pressure to afford 3-[[4-(2-amino-2-spiro[3.3]heptan-2-yl-ethoxy)-6-(2,6-dimethylphenyl)pyrimidin-2-yl]sulfamoyl]benzoic acid (hydrochloride salt) (2.24 g, 72%) as a white solid. ¹H NMR (400 MHz, DMSO-d₆) δ 8.44 (s, 1H), 8.27-8.06 (m, 5H), 7.70 (t, J=7.8 Hz, 1H), 7.31-7.21 (m, 1H), 7.13 (d, J 7.6 Hz, 2H), 6.30 (s, 1H), 4.28 (dd, J 11.6, 2.6 Hz, 1H), 4.15-3.97 (m, 1H), 3.50-3.34 (m, 1H), 2.43-2.25 (m, 1H), 2.14-1.70 (m, 16H). ESI-MS m/z calc. 536.2093, found 537.2 (M+1)⁺; Retention time: 2.49 minutes, LC method Y.

### Step 6: 3-[[4-[2-[[6-[Cyclobutyl(methyl)amino]pyrazin-2-yl]methylamino]-2-spiro[3.3]heptan-2-yl-ethoxy]-6-(2,6-dimethylphenyl)pyrimidin-2-yl]sulfamoyl]benzoic acid

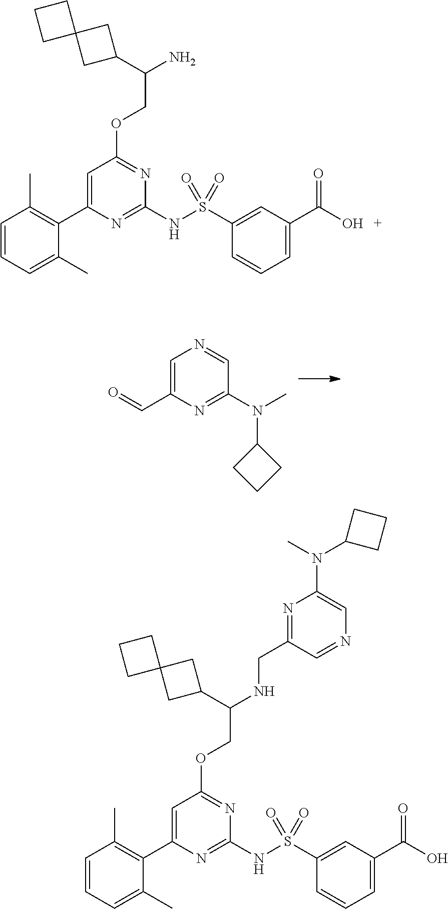

3-[[4-(2-Amino-2-spiro[3.3]heptan-2-yl-ethoxy)-6-(2,6-dimethylphenyl)pyrimidin-2-yl]sulfamoyl]benzoic acid (hydrochloride salt) (50 mg, 0.08349 mmol) and 6-[cyclobutyl(methyl)amino]pyrazine-2-carbaldehyde (18 mg, 0.08989 mmol) were combined in DCM (0.5 mL). acetic acid (7 μL, 0.1231 mmol) was added. The mixture was cooled to 0° C. before the addition of DIEA (32 μL, 0.1837 mmol) followed by sodium triacetoxyborohydride (88 mg, 0.4152 mmol). The reaction mixture was stirred at 0° C. for 30 minutes. The reaction was quenched with the addition of aqueous 1 M HCl (0.4 mL) and methanol (0.3 mL) and DMSO (0.5 mL). Purification by reverse phase HPLC (10-99% acetonitrile/5 mM aqueous HCl over 15 minutes) gave 3-[[4-[2-[[6-[cyclobutyl(methyl)amino]pyrazin-2-yl]methylamino]-2-spiro[3.3]heptan-2-yl-ethoxy]-6-(2,6-dimethylphenyl)pyrimidin-2-yl]sulfamoyl]benzoic acid (hydrochloride salt) (54 mg, 86%) as a yellow solid. ESI-MS m/z calc. 711.3203, found 712.7 (M+1)⁺; Retention time: 1.39 minutes; LC method A.

### Step 7: 12-[[6-[Cyclobutyl(methyl)amino]pyrazin-2-yl]methyl]-6-(2,6-dimethylphenyl)-2,2-dioxo-11-spiro[3.3]heptan-2-yl-9-oxa-2λ⁶-thia-3,5,12,19-tetrazatricyclo[12.3.1.14,8]nonadeca-1(18),4(19),5,7,14,16-hexaen-13-one, enantiomer 1 (Compound 1320) and 12-[[6-[cyclobutyl(methyl)amino]pyrazin-2-yl]methyl]-6-(2,6-dimethylphenyl)-2,2-dioxo-11-spiro[3.3]heptan-2-yl-9-oxa-2λ⁶-thia-3,5,12,19-tetrazatricyclo[12.3.1.14,8]nonadeca-1(18),4(19),5,7,14,16-hexaen-13-one, enantiomer 2 (Compound 1321)

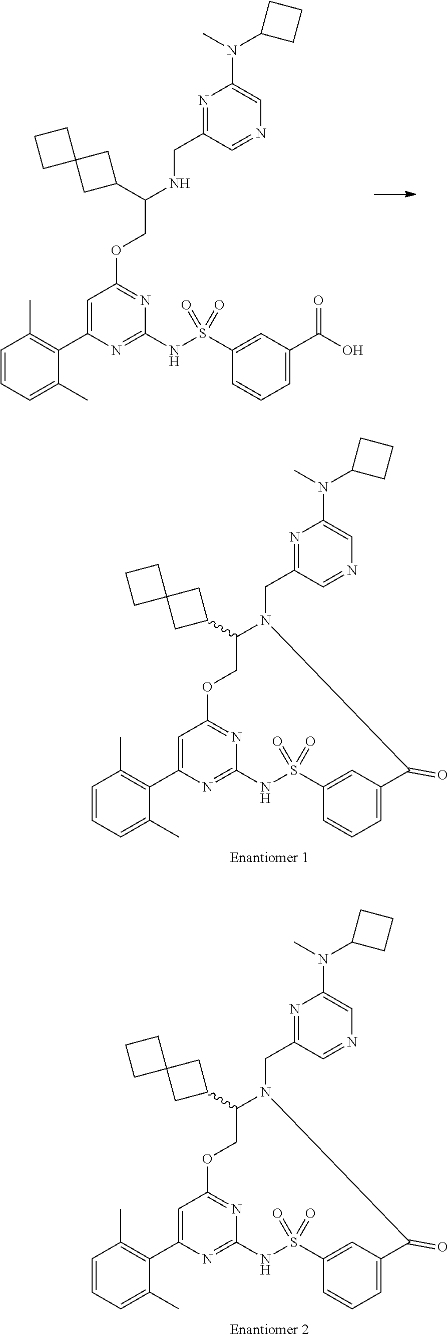 [SMILES: Cc1cccc(C)c1-c1cc(OCC(NCc2cncc(N(C)C3CCC3)n2)C2CC3(CCC3)C2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cncc(N(C)C3CCC3)n1)C(C1CC3(CCC3)C1)CO2 ; Cc1cccc(C)c1-c1cc2nc(n1)NS(=O)(=O)c1cccc(c1)C(=O)N(Cc1cncc(N(C)C3CCC3)n1)C(C1CC3(CCC3)C1)CO2]

3-[[4-[2-[[6-[Cyclobutyl(methyl)amino]pyrazin-2-yl]methylamino]-2-spiro[3.3]heptan-2-yl-ethoxy]-6-(2,6-dimethylphenyl)pyrimidin-2-yl]sulfamoyl]benzoic acid (hydrochloride salt) (54 mg, 0.07216 mmol) and 2-chloro-4,6-dimethoxy-1,3,5-triazine (19 mg, 0.1082 mmol) were combined in DMF (0.5 mL). The solution was cooled to 0° C. before the addition of N-methylmorpholine (40 μL, 0.3638 mmol). The reaction mixture was allowed to slowly warm to room temperature and stirred overnight. After filtration, purification by reverse phase HPLC (10-99% acetonitrile/5 mM aqueous HCl over 30 min) was performed. The diastereomers were separated by chiral SFC using a Phenomenex LUX-4 (21.2×250 mm, 5 μm) at 50° C. Mobile phase was 44% MeOH (20 mM NH₃), 56% CO₂ at a 70 mL/min flow. Concentration of the sample was 20.8 mg/mL in MeOH:DMSO (77:23), injection volume 650 μL with an outlet pressure of 185 bar, detection wavelength of 210 nm. This provided 12-[[6-[cyclobutyl(methyl) amino]pyrazin-2-yl]methyl]-6-(2,6-dimethylphenyl)-2,2-dioxo-11-spiro[3.3]heptan-2-yl-9-oxa-2λ⁶-thia-3,5,12,19-tetrazatricyclo[12.3.1.14,8]nonadeca-1(18),4(19),5,7,14,16-hexaen-13-one (6.2 mg, 25%) as Peak 1, enantiomer 1, as an orange solid, ¹H NMR (400 MHz, Chloroform-d) δ 8.77 (d, J 1.9 Hz, 1H), 8.11 (d, J 7.9 Hz, 1H), 7.93-7.87 (m, 3H), 7.69 (t, J 7.8 Hz, 1H), 7.21 (t, J 7.6 Hz, 1H), 7.07 (d, J 7.6 Hz, 2H), 6.16 (s, 1H), 5.43 (dd, J 11.4, 4.0 Hz, 1H), 5.12 (d, J 15.5 Hz, 1H), 4.56 (q, J 8.3 Hz, 1H), 4.17 (t, J 11.4 Hz, 1H), 4.03-3.91 (m, 2H), 2.62 (q, J 8.7 Hz, 1H), 2.28 (td, J 7.6, 3.1 Hz, 2H), 2.19 (dt, J 20.1, 10.1 Hz, 3H), 2.05 (s, 6H), 1.99 (t, J=6.6 Hz, 3H), 1.73 (dd, J 7.0, 3.1 Hz, 2H), 1.65 (dd, J 8.7, 4.4 Hz, 3H), 1.48 (d, J 6.4 Hz, 2H), 1.34-1.28 (m, 2H), 1.25 (s, 1H), 1.08-1.02 (m, 1H). ESI-MS m/z calc. 693.30975, found 694.5 (M+1)⁺; Retention time: 1.75 minutes and 12-[[6-[cyclobutyl(methyl)amino]pyrazin-2-yl]methyl]-6-(2,6-dimethylphenyl)-2,2-dioxo-11-spiro[3.3]heptan-2-yl-9-oxa-2?⁶-thia-3,5,12,19-tetrazatricyclo[12.3.1.14,8]nonadeca-1(18),4(19),5,7,14,16-hexaen-13-one (5.3 mg, 21%) as Peak 2, enantiomer 2, as an orange solid, ¹H NMR (400 MHz, Chloroform-d) δ 8.76 (d, J 1.8 Hz, 1H), 8.10 (d, J 7.9 Hz, 1H), 7.90 (d, J 5.8 Hz, 3H), 7.68 (t, J 7.8 Hz, 1H), 7.21 (t, J 7.6 Hz, 1H), 7.06 (d, J 7.6 Hz, 2H), 6.14 (s, 1H), 5.43 (dd, J 11.3, 4.0 Hz, 1H), 5.12 (d, J 15.5 Hz, 1H), 4.57 (h, J 8.4, 7.9 Hz, 1H), 4.15 (t, J 11.4 Hz, 1H), 4.01-3.93 (m, 2H), 2.66-2.56 (m, 1H), 2.33-2.25 (m, 2H), 2.20 (q, J 10.0 Hz, 2H), 2.04 (s, 6H), 1.98 (d, J 7.7 Hz, 3H), 1.73 (dt, J 6.1, 2.9 Hz, 2H), 1.66 (td, J 9.1, 7.9, 3.2 Hz, 3H), 1.53-1.46 (m, 2H), 1.32 (t, J 10.2 Hz, 2H), 1.26 (s, 1H), 1.06 (dd, J 11.0, 8.5 Hz, 1H), 0.90-0.84 (m, 1H). ESI-MS m/z calc. 693.30975, found 694.5 (M+1)⁺; Retention time: 1.75 minutes; LC method A.

### Example 162: Preparation of Compound 1322

### Step 1: Methyl 2-(benzyloxycarbonylamino)acetate

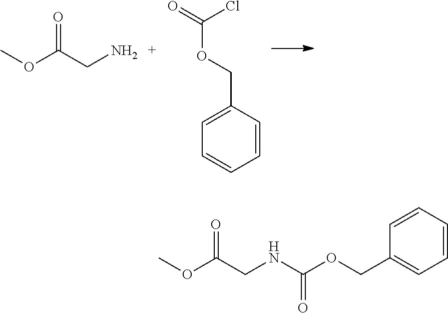

Sodium bicarbonate (15.88 g, 189.03 mmol) in solution in water (120 mL) was added dropwise over 5 minutes to a solution of methyl 2-aminoacetate (hydrochloride salt) (11.99 g, 95.497 mmol) and benzyl chloroformate (21.510 g, 18 mL, 126.09 mmol) in water (60 mL) at room temperature. The reaction was stirred at room temperature for 2 h. EtOAc (100 mL) was added, the phases were separated, and the aqueous layer was extracted with EtOAc (2×100 mL). The combined organic layers were dried over sodium sulfate, filtered and concentrated under reduced pressure. The residue was purified by silica gel chromatography (column: 120 g, 0-40% EtOAc in Heptanes) to afford methyl 2-(benzyloxycarbonylamino)acetate (20.037 g, 94%) as a colorless oil. ESI-MS m/z calc. 223.08446, found 224.2 (M+1)⁺; Retention time: 1.54 minutes; LC method X.

### Step 2: Methyl 2-(benzyloxycarbonylamino)-3-(dimethylamino)prop-2-enoate

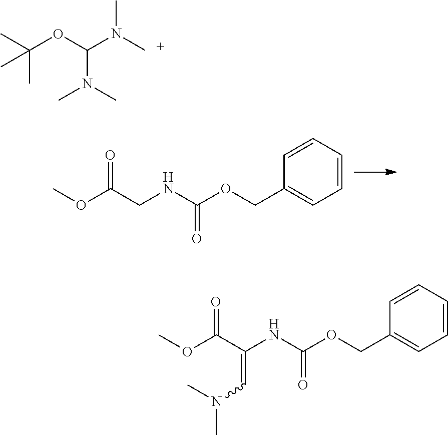

1-tert-Butoxy-N,N,N′,N′-tetramethyl-methanediamine (13.504 g, 16 mL, 77.483 mmol) was added to a solution of methyl 2-(benzyloxycarbonylamino)acetate (11.804 g, 52.827 mmol) in toluene (30 mL) at room temperature. The reaction was stirred and heated at 90° C. for 16 h. The solvent was evaporated to dryness. The residue was purified by silica gel chromatography (column: 275 g, 20-100% EtOAc in Heptanes) to afford methyl 2-(benzyloxycarbonylamino)-3-(dimethylamino)prop-2-enoate (12.649 g, 81%) as a beige solid. ESI-MS m/z calc. 278.12665, found 279.2 (M+1)⁺; Retention time: 2.66 minutes; LC method 1D.

### Step 3: Methyl 2-(benzyloxycarbonylamino)-3-[[(1S)-2-ethoxy-1-methyl-2-oxo-ethyl]amino]prop-2-enoate

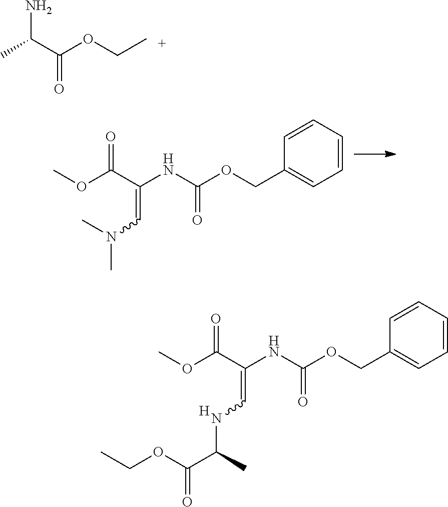

Methyl 2-(benzyloxycarbonylamino)-3-(dimethylamino)prop-2-enoate (8.146 g, 28.070 mmol) and ethyl (2S)-2-aminopropanoate (hydrochloride salt) (8.3 g, 54.034 mmol) were solubilized in anhydrous MeOH (50 mL) and stirred at 40° C. for 3 h. The solvent was evaporated to dryness. The residue was purified by silica gel chromatography (column: 120 g, 0-50% EtOAc in Heptanes) to afford a mixture of the 2 isomers of methyl 2-(benzyloxycarbonylamino)-3-[[(1S)-2-ethoxy-1-methyl-2-oxo-ethyl]amino]prop-2-enoate (8.915 g, 81%) as a pale orange oil. Major isomer: ESI-MS m/z calc. 350.1478, found 351.2 (M+1)⁺; Retention time: 1.65 minutes. Minor isomer ESI-MS m/z calc. 350.1478, found 351.2 (M+1)⁺; Retention time: 1.72 minutes; LC method X.

### Step 4: Methyl (2S)-2-methyl-3-oxo-2,4-dihydro-1H-pyrazine-5-carboxylate

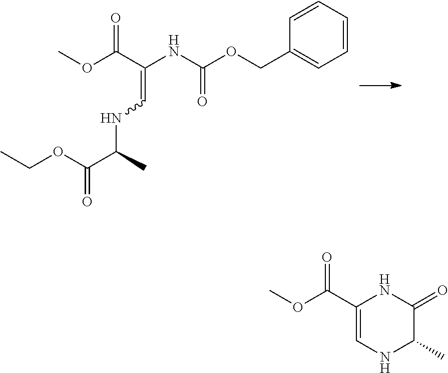

Palladium on carbon (6.73 g, 10% w/w, 6.3240 mmol) and ammonium formate (9.42 g, 149.39 mmol) were added to a solution of methyl 2-(benzyloxycarbonylamino)-3-[[(1S)-2-ethoxy-1-methyl-2-oxo-ethyl]amino]prop-2-enoate (12.91 g, 31.689 mmol) in EtOH (700 mL). The reaction was stirred at room temperature for 3 h. The mixture was filtered through celite. The filter cake was washed with ethanol (2×100 mL) then the solvent was removed under reduced pressure. The residue was purified by silica gel chromatography (column: 120 g, 20-100% EtOAc in Heptanes) to afford methyl (2S)-2-methyl-3-oxo-2,4-dihydro-1H-pyrazine-5-carboxylate (4.006 g, 74%) as an orange solid. ESI-MS m/z calc. 170.06914, found 171.2 (M+1)⁺; Retention time: 0.95 minutes; LC method X.

### Step 5: Methyl 5-methyl-6-oxo-1H-pyrazine-2-carboxylate

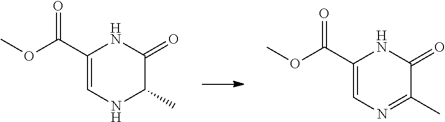

Palladium on carbon (3.72 g, 10% w/w, 3.4956 mmol) was added to a solution of methyl (2S)-2-methyl-3-oxo-2,4-dihydro-1H-pyrazine-5-carboxylate (4.006 g, 23.518 mmol) in xylenes (125 mL). The reaction was stirred at 100° C. for 16 h. The crude mixture was cooled to room temperature, diluted with methanol (50 mL) and then filtered through celite. The filter cake was washed with methanol (3×50 mL) then the solvent was removed under reduced pressure to afford methyl 5-methyl-6-oxo-1H-pyrazine-2-carboxylate (2.068 g, 52%) as an orange solid. ¹H NMR (400 MHz, CDCl₃) δ 7.93 (s, 1H), 3.99 (s, 3H), 2.56 (s, 3H), (1H missing, labile proton). ESI-MS m/z calc. 168.0535, found 169.2 (M+1)⁺; Retention time: 1.02 minutes; LC method X.

### Step 6: Methyl 6-chloro-5-methyl-pyrazine-2-carboxylate

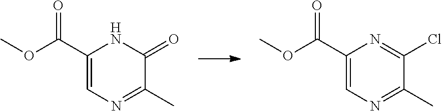

Phenyl dichlorophosphate (11.280 g, 8 mL, 53.464 mmol) was added to methyl 5-methyl-6-oxo-1H-pyrazine-2-carboxylate (2.068 g, 12.286 mmol) and the mixture was stirred at 170° C. for 1 h. The reaction mixture was cooled to room temperature, diluted with EtOAc (50 mL), washed with brine (2×50 mL), dried over sodium sulfate, filtered and concentrated under reduced pressure. The residue was purified by silica gel chromatography (column: 120 g, 0-50% EtOAc in Heptanes) to afford methyl 6-chloro-5-methyl-pyrazine-2-carboxylate (960 mg, 42%) as an orange solid. ESI-MS m/z calc. 186.0196, found 187.2 (M+1)⁺; Retention time: 1.44 minutes; LC method X.

### Step 7: (6-Chloro-5-methyl-pyrazin-2-yl)methanol

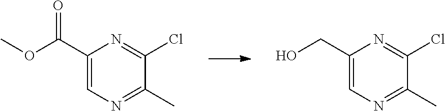

DIBAL in toluene (17 mL of 1 M, 17.000 mmol) was slowly added to a mixture of methyl 6-chloro-5-methyl-pyrazine-2-carboxylate (800 mg, 4.2873 mmol) in THF (40 mL) at −10° C. The reaction was stirred at −10° C. for 1 h. An aqueous saturated solution of Rochelle salt (20 mL) was slowly added, and the mixture was stirred at room temperature for 16 h. The product was extracted with DCM (2×10 mL). The combined organic layers were dried with sodium sulfate, filtered and concentrated under reduced pressure to afford (6-chloro-5-methyl-pyrazin-2-yl)methanol (677 mg, 99%) as a beige solid. ESI-MS m/z calc. 158.02469, found 159.2 (M+1)⁺; Retention time: 1.2 minutes; LC method X.

### Step 8: 6-Chloro-5-methyl-pyrazine-2-carbaldehyde

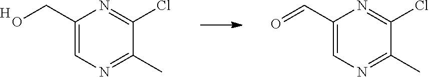

Dess-martin periodinane (2.93 g, 6.9081 mmol) was added to a solution of (6-chloro-5-methyl-pyrazin-2-yl)methanol (675 mg, 4.2521 mmol) in DCM (13 mL). The reaction was stirred at room temperature for 2 h. Pentane (50 ml) was added under stirring. The mixture was deposited on a silica gel pad (5 cm) and the product was filtrated using pentane (2×25 mL) and then EtOAc/heptanes (1:1) (50 ml) as eluent. The filtrate was evaporated under reduced pressure to afford 6-chloro-5-methyl-pyrazine-2-carbaldehyde (662 mg, 94%) as a yellow oil. ¹H NMR (400 MHz, CDCl₃) δ 10.07 (s, 1H), 8.96 (s, 1H), 2.79 (s, 3H). ESI-MS m/z calc. 156.00903, found 157.1 (M+1)⁺; Retention time: 2.21 minutes; LC method 1D.

### Step 9: 3-[[4-[(2R)-2-[(6-Chloro-5-methyl-pyrazin-2-yl)methylamino]-4,4-dimethyl-pentoxy]-6-(2,6-dimethylphenyl)pyrimidin-2-yl]sulfamoyl]benzoic acid

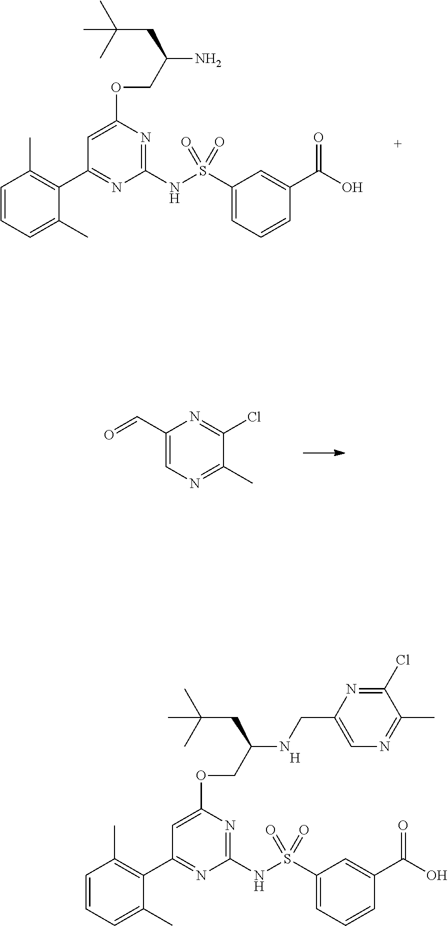

6-Chloro-5-methyl-pyrazine-2-carbaldehyde (399 mg, 2.4210 mmol) and grinded 3-[[4-[(2R)-2-amino-4,4-dimethyl-pentoxy]-6-(2,6-dimethylphenyl)pyrimidin-2-yl]sulfamoyl]benzoic acid (hydrochloride salt) (1.15 g, 2.0253 mmol) were stirred together in DCM (20 mL) for 30 minutes at room temperature. The reaction was cooled down at 0° C. and sodium triacetoxyborohydride (990 mg, 4.6711 mmol) was added. The reaction was stirred from 0° C. to room temperature for 2 h. The reaction was quenched with an aqueous saturated solution of NH₄Cl (10 mL) and the phases were separated. The aqueous layer was extracted with EtOAc (2×25 mL). The combined organic layers were washed with brine (15 mL), dried over sodium sulfate, filtered, and concentrated under reduced pressure. The crude was purified by reversed-phase chromatography (Column: C₁₈. Gradient: 50-100% methanol in water) to afford 3-[[4-[(2R)-2-[(6-chloro-5-methyl-pyrazin-2-yl)methylamino]-4,4-dimethyl-pentoxy]-6-(2,6-dimethylphenyl)pyrimidin-2-yl]sulfamoyl]benzoic acid (649 mg, 41%) as a white solid. ESI-MS m/z calc. 652.22345, found 653.2 (M+1)⁺; Retention time: 1.45 minutes; LC method X.

### Step 10: (11R)-12-[(6-Chloro-5-methyl-pyrazin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one

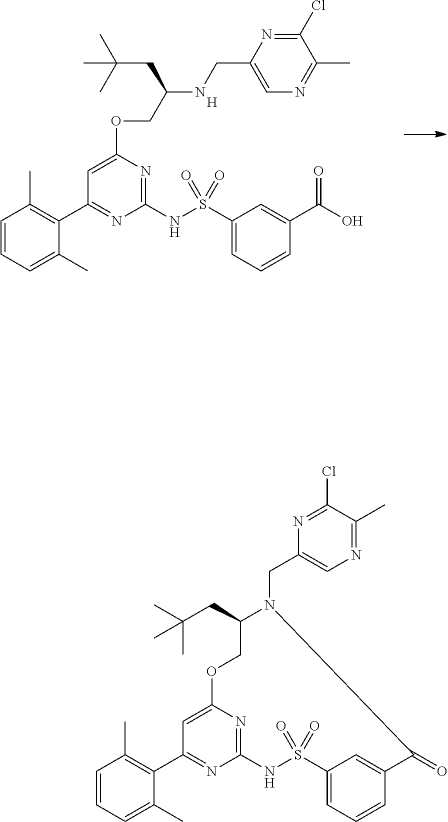

N-Methylmorpholine (1.5640 g, 1.7 mL, 15.463 mmol) followed by 2-chloro-4,6-dimethoxy-1,3,5-triazine (430 mg, 2.4491 mmol) were added to a solution of 3-[[4-[(2R)-2-[(6-chloro-5-methyl-pyrazin-2-yl)methylamino]-4,4-dimethyl-pentoxy]-6-(2,6-dimethylphenyl)pyrimidin-2-yl]sulfamoyl]benzoic acid (918 mg, 1.2115 mmol) in DMF (100 mL) at 0° C. The reaction was stirred from 0° C. to room temperature for 16 h. The solvent was evaporated to dryness. The residue was purified by reversed-phase chromatography (Column: C₁₈. Gradient: 50-100% methanol in water). The pure fractions were concentrated in vacuo and lyophilized to afford (11R)-12-[(4-chloro-5-methyl-pyrimidin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (552 mg, 68%) as a white solid. ¹H NMR (400 MHz, DMSO-d₆) δ 13.09 (br. s., 1H), 8.65 (s, 1H), 8.61 (br. s., 1H), 7.94 (br. s., 1H), 7.66 (br. s., 2H), 7.26 (t, J 7.3 Hz, 1H), 7.12 (d, J 6.6 Hz, 2H), 6.42 (br. s., 1H), 5.32 (dd, J 10.8, 4.2 Hz, 1H), 4.83 (d, J 16.1 Hz, 1H), 4.65 (d, J 15.9 Hz, 1H), 4.31 (t, J 11.0 Hz, 1H), 4.05-3.92 (m, 1H), 2.59 (s, 3H), 2.25-1.87 (m, 6H), 1.82 (dd, J 15.3, 8.7 Hz, 1H), 1.43 (d, J 14.9 Hz, 1H), 0.56 (s, 9H). ESI-MS m/z calc. 634.2129, found 635.3 (M+1)⁺; Retention time: 4.5 minutes; LC method Y.

### Step 11: (11R)-6-(2,6-Dimethylphenyl)-11-(2,2-dimethylpropyl)-12-[[6-[isobutyl(methyl)amino]-5-methyl-pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1322)

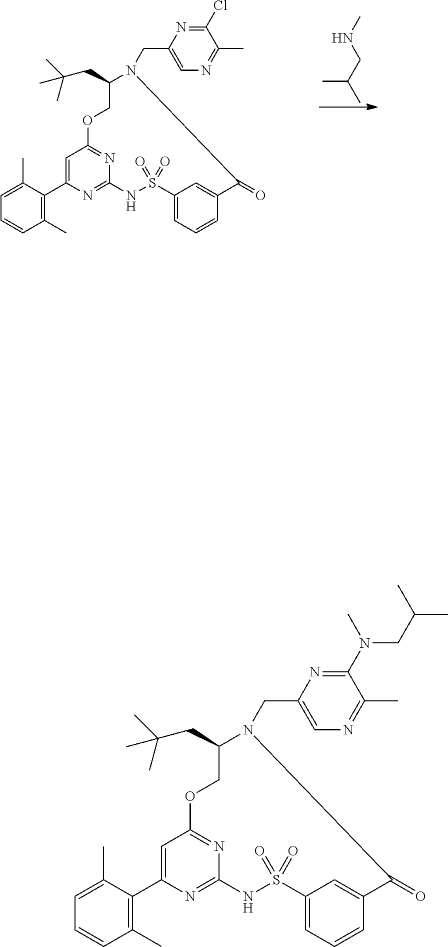

A 4 mL vial was charged with (11R)-12-[(6-chloro-5-methyl-pyrazin-2-yl)methyl]-6-(2,6-dimethylphenyl)-11-(2,2-dimethylpropyl)-2,2-dioxo-9-oxa-2⁶-thia-3, 5,12,19-tetrazatricyclo[12.3.1.14,8]nonadeca-1(18),4(19),5,7,14,16-hexaen-13-one (41.7 mg, 0.06565 mmol), N,2-dimethylpropan-1-amine (23 mg, 0.2639 mmol), potassium carbonate (75 mg, 0.5427 mmol) (powdered, 325 mesh), DMSO (200 μL) and dioxane (100 μL). The vial was purged with nitrogen, capped and stirred at 120° C. for 3 days. After cooling down to room temperature, the reaction was diluted with DMSO (1 mL), filtered and purified by reverse phase HPLC (1-99% acetonitrile/5 mM aqueous HCl over 25 min). After evaporation, the residue was triturated with DCM/hexanes and the solvents evaporated to give (11R)-6-(2,6-dimethylphenyl)-11-(2,2-dimethylpropyl)-12-[[6-[isobutyl(methyl)amino]-5-methyl-pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (26 mg, 58%) as a yellow solid. ¹H NMR (400 MHz, Chloroform-d) δ 8.79 (t, J 1.9 Hz, 1H), 8.33 (broad s, 1H), 8.10 (s, 1H), 8.01 (d, J 8.0 Hz, 1H), 7.86 (d, J 7.7 Hz, 1H), 7.63 (t, J 7.8 Hz, 1H), 7.21 (t, J 7.6 Hz, 1H), 7.07 (d, J 7.6 Hz, 2H), 6.28 (s, 1H), 5.49 (dd, J 10.3, 3.3 Hz, 1H), 5.02 (d, J 15.1 Hz, 1H), 4.28-4.03 (m, 3H), 3.22-3.06 (m, 2H), 3.02 (s, 3H), 2.55 (s, 3H), 2.10-1.92 (m, 7H), 1.71 (dd, J 15.0, 8.2 Hz, 1H), 1.55 (d, J 15.1 Hz, 1H), 0.87-0.78 (m, 6H), 0.61 (s, 9H). ESI-MS m/z calc. 685.341, found 686.82 (M+1)⁺; Retention time: 1.89 minutes; LC method A.

### Example 163: Preparation of Compound 1323

### Step 1: 3-[[4-[(3R,4R)-4-Amino-1-tert-butoxycarbonyl-pyrrolidin-3-yl]oxy-6-(2,6-dimethylphenyl)pyrimidin-2-yl]sulfamoyl]benzoic acid

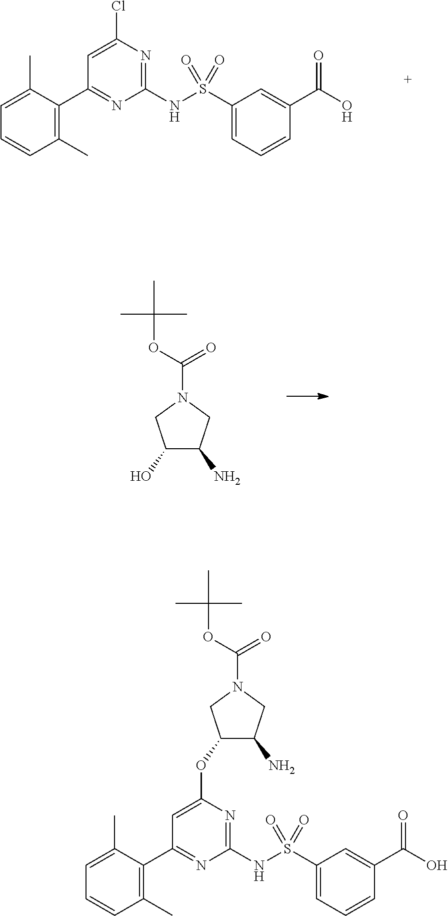 [SMILES: Cc1cccc(C)c1-c1cc(Cl)nc(NS(=O)(=O)c2cccc(C(=O)O)c2)n1 ; CC(C)(C)OC(=O)N1C[C@@H](N)[C@H](O)C1 ; Cc1cccc(C)c1-c1cc(O[C@@H]2CN(C(=O)OC(C)(C)C)C[C@H]2N)nc(NS(=O)(=O)c2cccc(C(=O)O)c2)n1]

A solution of 3-[[4-chloro-6-(2,6-dimethylphenyl)pyrimidin-2-yl]sulfamoyl]benzoic acid (10.6 g, 25.37 mmol), tert-butyl (3R,4R)-3-amino-4-hydroxy-pyrrolidine-1-carboxylate (5.2 g, 25.71 mmol), and sodium t-butoxide (7.3 g, 75.96 mmol) in THF (0.13 L) was stirred for 18 hours. The reaction was acidified with 1 M citric acid, diluted with water, and extracted with ethyl acetate. The combined extracts were washed with brine, dried over sodium sulfate, and evaporated under vacuum to give a tan oil. The oil was stirred with diethyl ether to give a colorless solid. The solid was filtered, washed with diethyl ether, and dried under vacuum to give 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (15.2 g, 103%) ESI-MS m/z calc. 583.2101, found 584.3 (M+1)⁺; Retention time: 0.49 minutes; LC method D.

### Step 2: 3-[[4-[(3R,4R)-1-tert-Butoxycarbonyl-4-[[6-[cyclobutyl(methyl) amino]pyrazin-2-yl]methylamino]pyrrolidin-3-yl]oxy-6-(2,6-dimethyl phenyl)pyrimidin-2-yl]sulfamoyl]benzoic acid

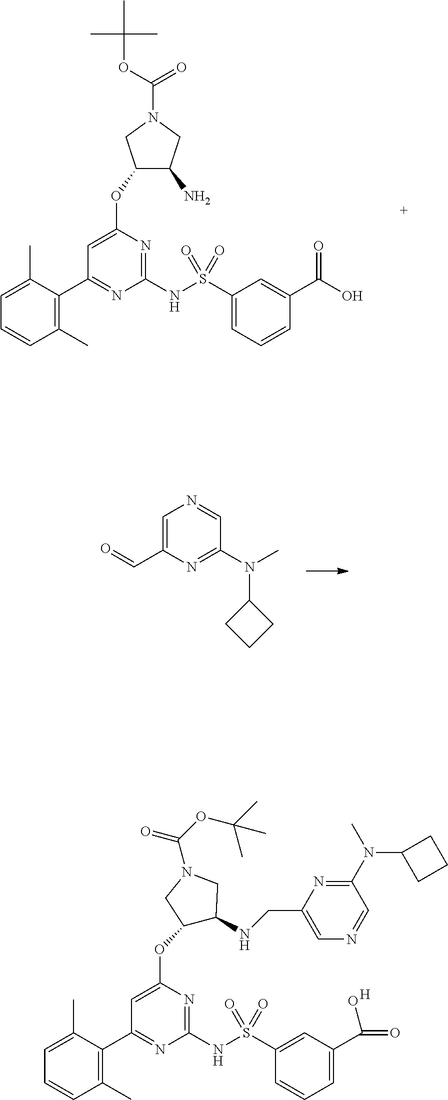

In a 4 mL vial, to a stirred mixture of 3-[[4-[(3R,4R)-4-amino-1-tert-butoxycarbonyl-pyrrolidin-3-yl]oxy-6-(2,6-dimethylphenyl)pyrimidin-2-yl]sulfamoyl]benzoic acid (200 mg, 0.3427 mmol) and 6-[cyclobutyl(methyl)amino]pyrazine-2-carbaldehyde (70 mg, 0.3661 mmol) in anhydrous dichloromethane (1.5 mL) were added glacial acetic acid (20 μL, 0.3517 mmol) and DIPEA (100 μL, 0.5741 mmol), in that order, at 0° C. under nitrogen. After 2-3 min, sodium triacetoxyborohydride (225 mg, 1.062 mmol) was added in one portion to the yellow solution. The heterogeneous reaction was stirred at that temperature for 15 min. Then the reaction was quenched with 1 M hydrochloric acid (1.2 mL), MeOH (1 mL) and DMSO (1 mL) and the resulting solution was filtered. After preparative reverse-phase HPLC [1-99% acetonitrile inm water (containing 5 mM HCl) over 15 min], the desired fraction was extracted with ethyl acetate (3×15 mL). the combined organics were washed with brine, dried over sodium sulfate, filtered and concentrated under reduced pressure to furnish 3-[[4-[(3R,4R)-1-tert-butoxycarbonyl-4-[[6-[cyclobutyl(methyl)amino]pyrazin-2-yl]methylamino]pyrrolidin-3-yl]oxy-6-(2,6-dimethylphenyl)pyrimidin-2-yl]sulfamoyl]benzoic acid (hydrochloride salt) (152 mg, 56%) as a yellow solid. ESI-MS m/z calc. 758.32104, found 759.2 (M+1)⁺; Retention time: 1.43 minutes; LC method A.

### Step 3: tert-Butyl (3R,7R)-8-[[6-[cyclobutyl(methyl)amino]pyrazin-2-yl]methyl]-19-(2,6-dimethylphenyl)-9,15,15-trioxo-2-oxa-15λ⁶-thia-5,8,16,18,21-pentazatetracyclo[15.3.1.110,14.03,7]docosa-1(20),10,12,14(22),17(21),18-hexaene-5-carboxylate

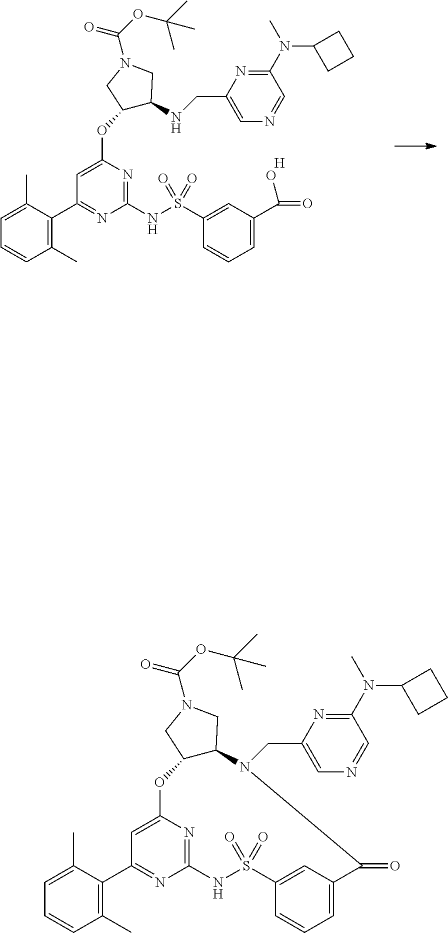

To a stirred solution of 3-[[4-[(3R,4R)-1-tert-butoxycarbonyl-4-[[6[cyclobutyl(methyl)amino]pyrazin-2-yl]methylamino]pyrrolidin-3-yl]oxy-6-(2,6-dimethylphenyl)pyrimidin-2-yl]sulfamoyl]benzoic acid (hydrochloride salt) (193 mg, 0.2427 mmol) in anhydrous DMF (10 mL) was added CDMT (80 mg, 0.4557 mmol) (CDMT), followed by addition of 4-methylmorpholine (200 μL, 1.819 mmol) at 0-4° C. (ice-water bath) under nitrogen. The light-yellow reaction was allowed to stir at that temperature for 5 min, then allowed to stir at room temperature for 15 h (overnight). After removing most of the volatiles under reduced pressure, the residue was purified by preparative reverse-phase HPLC (1-99% acetonitrile in water over 15 min, 5 mM HCl as modifier) to furnish tert-butyl (3R,7R)-8-[[6-[cyclobutyl(methyl)amino]pyrazin-2-yl]methyl]-19-(2,6-dimethylphenyl)-9,15,15-trioxo-2-oxa-15λ⁶-thia-5,8,16,18,21-pentazatetracyclo[15.3.1.110,14.03,7]docosa-1(20),10,12,14(22),17(21),18-hexaene-5-carboxylate (86 mg, 48%) as a yellow solid. ESI-MS m/z calc. 740.3104, found 741.3 (M+1)⁺; Retention time: 1.72 minutes; LC method A.

### Step 4: (3R,7R)-8-[[6-[Cyclobutyl(methyl)amino]pyrazin-2-yl]methyl]-19-(2,6-dimethylphenyl)-15,15-dioxo-2-oxa-15λ⁶-thia-5,8,16,18,21-pentazatetracyclo[15.3.1.110,14.03,7]docosa-1(20),10,12,14(22),17(21),18-hexaen-9-one

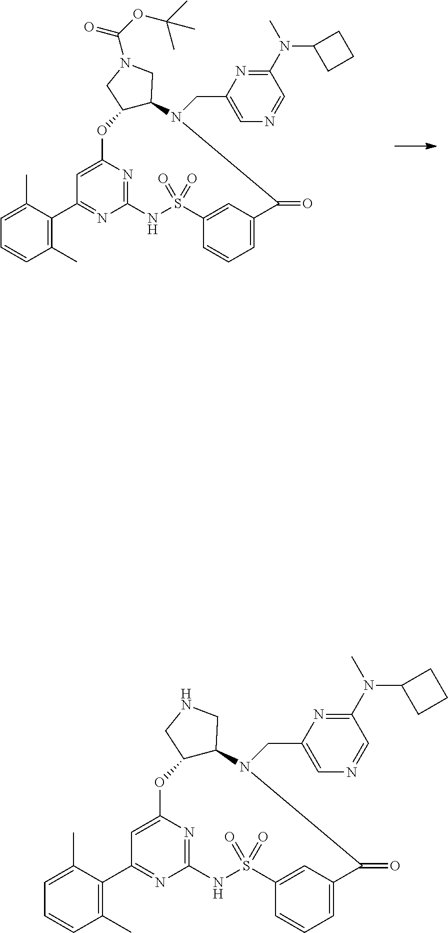 [SMILES: Cc1cccc(C)c1-c1cc2nc(n1)NS(=O)(=O)c1cccc(c1)C(=O)N(Cc1cncc(N(C)C3CCC3)n1)[C@@H]1CN(C(=O)OC(C)(C)C)C[C@H]1O2 ; Cc1cccc(C)c1-c1cc2nc(n1)NS(=O)(=O)c1cccc(c1)C(=O)N(Cc1cncc(N(C)C3CCC3)n1)[C@@H]1CNC[C@H]1O2]

To a solution of tert-butyl (3R,7R)-8-[[6-[cyclobutyl(methyl)amino]pyrazin-2-yl]methyl]-19-(2,6-dimethylphenyl)-9,15,15-trioxo-2-oxa-15λ⁶-thia-5,8,16,18,21-pentazatetracyclo[15.3.1.110,14.03,7]docosa-1(20),10,12,14(22),17(21),18-hexaene-5-carboxylate (88 mg, 0.1188 mmol) in anhydrous dichloromethane (1 mL) was added hydrogen chloride in dioxane (1.0 mL of 4.0 M, 4.000 mmol) (in dioxane) was stirred for 2 h under nitrogen. The volatiles were removed under reduced pressure. Upon further drying under vacuum (3R,7R)-8-[[6-[cyclobutyl(methyl)amino]pyrazin-2-yl]methyl]-19-(2,6-dimethylphenyl)-15,15-dioxo-2-oxa-15λ⁶-thia-5,8,16,18,21-pentazatetracyclo[15.3.1.110,14.03,7]docosa-1(20),10,12,14(22),17(21),18-hexaen-9-one (hydrochloride salt) (78 mg, 97%) was obtained as a yellow solid. ESI-MS m/z calc. 640.258, found 641.1 (M+1)⁺; Retention time: 0.4 minutes; LC method D.

### Step 5: (3R,7R)-8-[[6-[Cyclobutyl(methyl)amino]pyrazin-2-yl]methyl]-19-(2,6-dimethylphenyl)-15,15-dioxo-5-spiro[3.4]octan-2-yl-2-oxa-15λ⁶-thia-5,8,16,18,21-pentazatetracyclo[15.3.1.110,14.03,7]docosa-1(20),10,12,14(22),17(21),18-hexaen-9-one (Compound 1323)

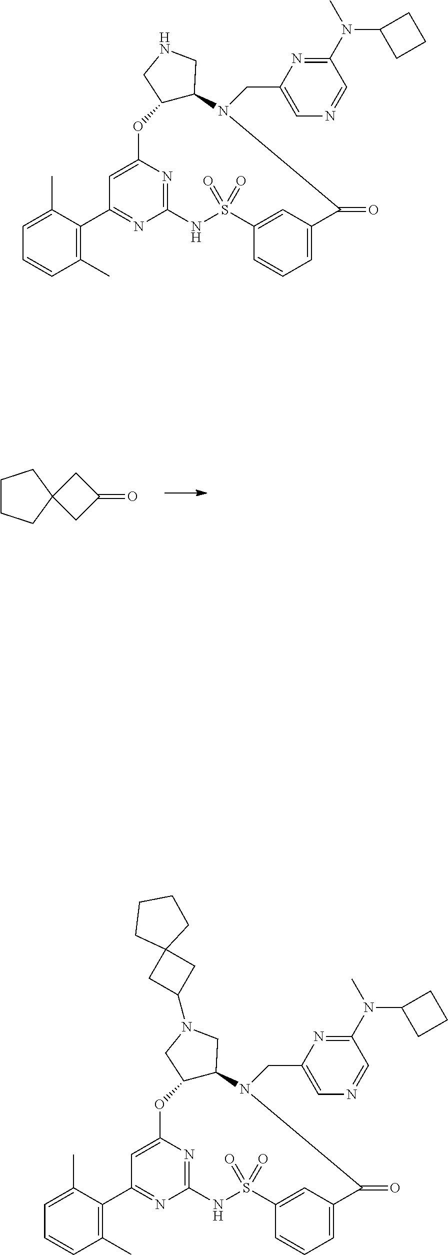 [SMILES: Cc1cccc(C)c1-c1cc2nc(n1)NS(=O)(=O)c1cccc(c1)C(=O)N(Cc1cncc(N(C)C3CCC3)n1)[C@@H]1CNC[C@H]1O2 ; O=C1CC2(CCCC2)C1 ; Cc1cccc(C)c1-c1cc2nc(n1)NS(=O)(=O)c1cccc(c1)C(=O)N(Cc1cncc(N(C)C3CCC3)n1)[C@@H]1CN(C3CC4(CCCC4)C3)C[C@H]1O2]

In a 4 mL vial, to a stirred mixture of (3R,7R)-8-[[6-[cyclobutyl(methyl)amino]pyrazin-2-yl]methyl]-19-(2,6-dimethylphenyl)-15,15-dioxo-2-oxa-15λ⁶-thia-5,8,16,18,21-pentazatetracyclo[15.3.1.110,14.03,7]docosa-1(20),10,12,14(22),17(21),18-hexaen-9-one (hydrochloride salt) (14 mg, 0.02067 mmol) and spiro[3.4]octan-2-one (11 mg, 0.08858 mmol) in anhydrous dichloromethane (150 μL) was added glacial acetic acid (5 μL, 0.08792 mmol) at ambient temperature. After stirring for 2-3 min, sodium triacetoxyborohydride (25 mg, 0.1180 mmol) was added at that temperature. The heterogeneous reaction was stirred at that temperature for 2 h. Then the reaction was quenched with aqueous 1M HCl 0.30 mL), MeOH (0.3 mL) and DMSO (0.5 mL) and purified by preparative reverse-phase HPLC [1-99% acetonitrile in water (containing 5 mM HCl) over 15 min] to furnish (3R,7R)-8-[[6-[cyclobutyl(methyl)amino] pyrazin-2-yl]methyl]-19-(2,6-dimethylphenyl)-15,15-dioxo-5-spiro[3.4]octan-2-yl-2-oxa-15λ⁶-thia-5,8,16,18,21-pentazatetracyclo[15.3.1.110,14.03,7]docosa-1(20),10,12,14(22),17(21),18-hexaen-9-one (hydrochloride salt) (9 mg, 55%) as a yellow solid. ¹H NMR (400 MHz, MeOD) δ 8.59 (s, 1H), 8.03 (s, 1H), 8.00-7.96 (m, 1H), 7.94 (s, 1H), 7.72 (d, J 7.6 Hz, 1H), 7.66 (t, J 7.7 Hz, 1H), 7.29 (t, J 7.6 Hz, 1H), 7.16 (d, J 7.7 Hz, 2H), 6.41 (s, 1H), 6.33 (d, J 2.5 Hz, 1H), 5.14 (d, J 16.7 Hz, 1H), 4.65 (p, J 8.4 Hz, 1H), 4.49 (d, J 16.7 Hz, 1H), 4.30 (d, J 14.2 Hz, 1H), 4.23-4.05 (m, 2H), 3.78 (d, J 14.2 Hz, 1H), 3.72 (dd, J 14.3, 7.1 Hz, 1H), 3.20 (s, 3H), 2.40-2.24 (m, 8H), 2.13 (s, 6H), 1.83-1.72 (m, 2H), 1.72-1.62 (m, 5H), 1.61-1.52 (m, 4H) ESI-MS m/z calc. 748.3519, found 749.3 (M+1)⁺; Retention time: 1.44 minutes; LC method A.

### Example 164: Preparation of Compound 1324

### Step 1: 04-Benzyl 03-tert-butyl (4R)-2-oxooxathiazolidine-3,4-dicarboxylate

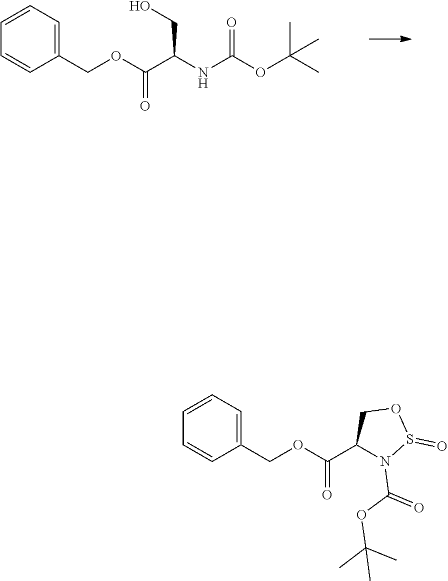

A solution of benzyl (2R)-2-(tert-butoxycarbonylamino)-3-hydroxy-propanoate (20.13 g, 68.161 mmol) in EtOAc (50 mL) was added dropwise over 5 minutes to a solution of thionyl chloride (16.310 g, 10 mL, 137.09 mmol) in EtOAc (400 mL) at −15° C. The reaction was stirred at −15° C. for 5 minutes. Pyridine (26.406 g, 27 mL, 333.83 mmol) was added dropwise over 5 minutes to the reaction at −15° C. The reaction was stirred from −15° C. to room temperature for 4 h. Water (50 mL) was added to the reaction mixture. The phases were split and the organic layer was washed with an aqueous 1N solution of HCl (25 mL), water (25 mL). The organic layer was dried with sodium sulfate, filtered, and concentrated under reduced pressure to afford a mixture of the diastereomers of 04-benzyl 03-tert-butyl (4R)-2-oxooxathiazolidine-3,4-dicarboxylate (23.689 g, 87%) as an orange oil. ESI-MS m/z calc. 341.09332, Retention time: 1.82 minutes; LC method X.

### Step 2: 04-Benzyl 03-tert-butyl (4R)-2,2-dioxooxathiazolidine-3,4-dicarboxylate

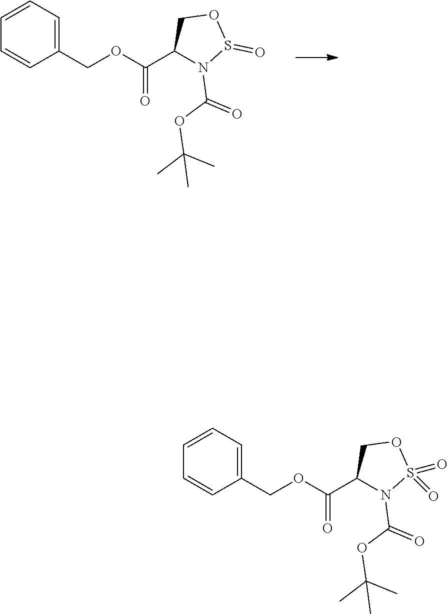

A solution of sodium periodate (17.7 g, 82.752 mmol) and ruthenium(III) chloride hydrate (1.34 g, 5.9438 mmol) in water (285 mL) was added dropwise over 2 minutes to a solution of 04-benzyl 03-tert-butyl (4R)-2-oxooxathiazolidine-3,4-dicarboxylate (23.689 g, 58.983 mmol) in MeCN (110 mL) at 0° C. The reaction was stirred from 0° C. to room temperature for 2 h. An aqueous 10% solution of sodium carbonate (100 mL), water (100 mL) and ethyl acetate (100 mL) were added to the reaction mixture and was filtered. The phases were split and the aqueous layer was extracted with EtOAc (2×10 mL). The combined organic layers were washed with brine (100 mL), dried with sodium sulfate, filtered, and concentrated under reduced pressure. The residue from filtration before work-up was suspended in EtOAc (200 mL) and was stirred at room temperature for 2 h. The suspension was filtered, and the filtrate was concentrated under reduced pressure. Products from work-up and from filtration were solubilized in EtOAc (50 mL each), combined and evaporated to dryness to afford 04-benzyl 03-tert-butyl (4R)-2,2-dioxooxathiazolidine-3,4-dicarboxylate (21.79 g, 89%) as an off-white solid. ESI-MS m/z calc. 357.0882, found 375.2 (M+18)⁺; Retention time: 1.82 minute; LC method X.

### Step 3: Benzyl (2R)-2-amino-3-isopropoxy-propanoate

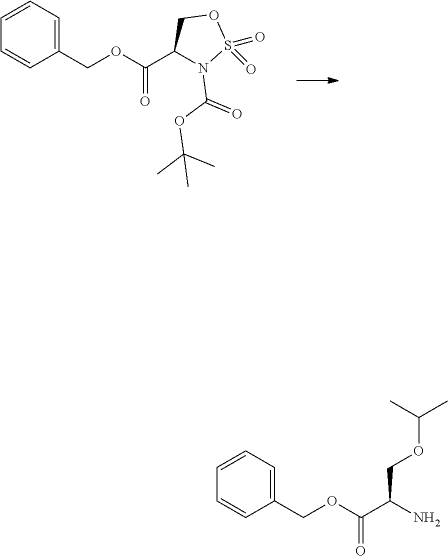 [SMILES: CC(C)(C)OC(=O)N1[C@@H](C(=O)OCc2ccccc2)COS1(=O)=O ; CC(C)OC[C@@H](N)C(=O)OCc1ccccc1]

04-Benzyl 03-tert-butyl (4R)-2,2-di oxooxathiazolidine-3,4-dicarboxylate (21.79 g, 52.375 mmol) was solubilized in iPrOH (250 mL) and heated to 80° C. for 16 h. The reaction mixture was cooled down to room temperature. EtOAc (100 mL), aqueous saturated solution of sodium bicarbonate (50 mL) and brine (50 mL) were added to the reaction mixture. The phases were split and the aqueous layer was extracted with EtOAc (2×100 mL). The combined organic layers were dried with sodium sulfate, filtered, and concentrated under reduced pressure. The residue was purified by silica gel chromatography (column: 220 g, 0-5% MeOH in DCM) to afford benzyl (2R)-2-amino-3-isopropoxy-propanoate (12.781 g, 67%) as an off-white solid. ¹H NMR (400 MHz, CDCl₃) δ 7.41-7.29 (m, 5H), 5.28 (d, J 12.0 Hz, 1H), 5.16 (d, J 12.2 Hz, 1H), 4.45 (br. s., 2H), 4.02 (t, J 3.7 Hz, 1H), 3.86-3.76 (m, 2H), 3.56 (spt, J 6.1 Hz, 1H), 1.12 (d, J 6.1 Hz, 3H), 1.05 (d, J 6.1 Hz, 3H). ESI-MS m/z calc. 237.13649, found 238.2 (M+1)⁺; Retention time: 1.24 minutes; LC method X.

### Step 4: (2R)-2-Amino-3-isopropoxy-propanoic acid

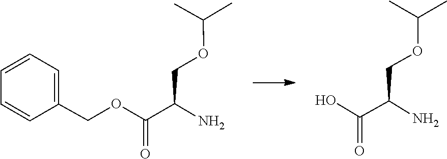

MeOH (125 mL) was added to benzyl (2R)-2-amino-3-isopropoxy-propanoate (12.781 g, 47.398 mmol). The reaction was sparged with nitrogen for 10 minutes and palladium on carbon (3.01 g, 2.8284 mmol) was added. The suspension was sparged with hydrogen for 5 minutes then stirred under a hydrogen atmosphere for 16 hour. The suspension was sparged with nitrogen for 10 minutes then filtered through a short pad of Celite which was washed with MeOH (100 mL). The solvent was removed under reduced pressure to afford crude (2R)-2-amino-3-isopropoxy-propanoic acid (8.89 g, 83%) as a white solid that was used in the following step without further purification. ¹H NMR (400 MHz, DMSO-d₆) δ 8.34 (br. s, 2H), 3.82-3.76 (m, 1H), 3.76-3.63 (m, 2H), 3.58 (spt, J 6.1 Hz, 1H), 1.13-1.05 (m, 6H, overlapped with impurity), (1H missing, labile proton). ESI-MS m/z calc. 147.08954, found 148.2 (M+1)⁺; Retention time: 0.25 minutes; LC method X.

### Step 5: (2R)-2-(Benzyloxycarbonylamino)-3-isopropoxy-propanoic acid

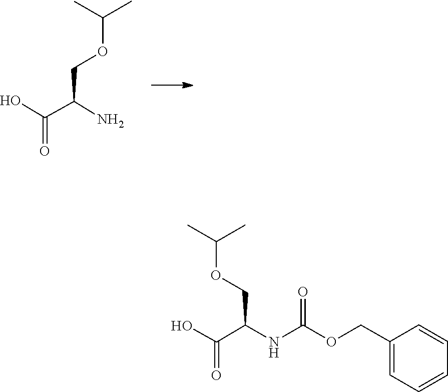

To a solution of (2R)-2-amino-3-isopropoxy-propanoic acid (8.89 g, 39.264 mmol) in anhydrous THF (150 mL) under nitrogen atmosphere at 0° C. were added N-(benzyloxycarbonyloxy)succinimide (21.15 g, 84.865 mmol) and triethylamine (21.054 g, 29 mL, 208.06 mmol). The reaction was then allowed to reach room temperature and stirred at room temperature for 16 h. Water (50 mL) and aqueous 1N HCl (200 mL) were added to the reaction mixture. The aqueous layer was extracted with EtOAc (3×100 mL). The combined organic layers were dried with sodium sulfate, filtered and concentrated under reduced pressure. The crude was purified by silica gel chromatography (column: 120 g, 0-10% MeOH in DCM). The fractions containing the product were combined and evaporated to dryness. The residue was purified by reversed-phase chromatography (Column: C₁₈. Gradient: 5-100% methanol in water) to afford (2R)-2-(benzyloxycarbonylamino)-3-isopropoxy-propanoic acid (2.853 g, 17%) as a pale yellow oil. ESI-MS m/z calc. 281.1263, found 282.2 (M+1)⁺; Retention time: 1.61 minutes; LC method X.

### Step 6: Benzyl N-[(1S)-1-(hydroxymethyl)-2-isopropoxy-ethyl]carbamate

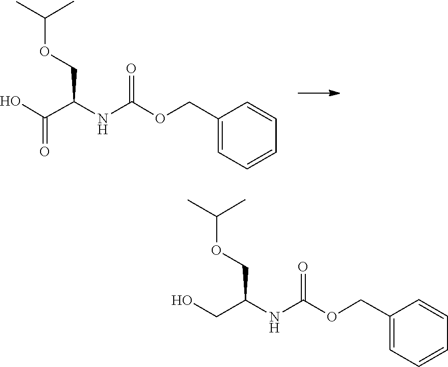

N-Methylmorpholine (1.7480 g, 1.9 mL, 17.282 mmol) and isobutyl chloroformate (2.4219 g, 2.3 mL, 17.733 mmol) were added to a solution of (2R)-2-(benzyloxycarbonylamino)-3-isopropoxy-propanoic acid (2.853 g, 6.5923 mmol) in DME (30 mL) at −15° C. The reaction was stirred at −15° C. for 2 hours. The precipitate was filtered and washed with DME (2 mL). The filtrate was transferred to a round bottom flask, cooled to 0° C. and sodium borohydride (1.07 g, 28.283 mmol) in solution in water (30 mL) was slowly added over 2 minutes. The reaction mixture was stirred at 0° C. for 1 h. More sodium borohydride (300 mg, 0.3175 mL, 7.9297 mmol) was added and the reaction was stirred at 0° C. for 1 h. Aqueous 1N solution of HCl (20 mL) was slowly added over 2 minutes to the reaction and the mixture was stirred at room temperature for 45 minutes. The phases were split and the aqueous layer was extracted with EtOAc (2×25 mL). The combined organic layers were dried with sodium sulfate, filtered and concentrated under reduced pressure. The residue was purified by silica gel chromatography (column: 12 g, 0-10% MeOH in DCM). The fractions containing the product were combined and evaporated to dryness. The residue was purified by reversed-phase chromatography (Column: C₁₈. Gradient: 5-100% methanol in water) to afford benzyl N-[(1S)-1-(hydroxymethyl)-2-isopropoxy-ethyl]carbamate (1.019 g, 55%) as a colorless oil. ESI-MS m/z calc. 267.14706, found 268.2 (M+1)⁺; Retention time: 1.58 minutes; LC method X.

### Step 7: (2S)-2-Amino-3-isopropoxy-propan-1-ol

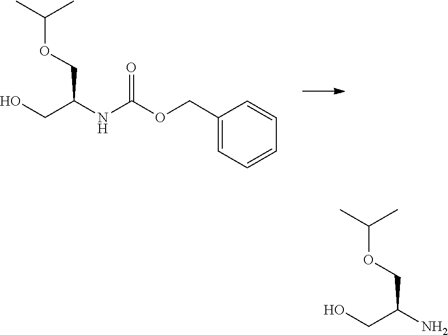

MeOH (10 mL) was added to benzyl N-[(1S)-1-(hydroxymethyl)-2-isopropoxy-ethyl]carbamate (969 mg, 3.4219 mmol). The reaction was degassed with nitrogen for 10 minutes and palladium on carbon (365 mg, 0.3430 mmol) was added. The suspension was sparged with hydrogen for 5 minutes then stirred under a hydrogen atmosphere for 16 hours. The suspension was sparged with nitrogen for 10 minutes then filtered through a short pad of Celite which was washed with MeOH (100 mL). The solvent was removed under reduced pressure to afford crude (2S)-2-amino-3-isopropoxy-propan-1-ol (449 mg, 94%) as a colorless oil that was used in the following step without further purification. ¹H NMR (400 MHz, CDCl₃) δ 3.64-3.49 (m, 3H), 3.48-3.43 (m, 1H), 3.42-3.34 (m, 1H), 3.05 (quin, J 5.3 Hz, 1H), 2.53-2.37 (m, 3H), 1.15 (d, J 6.1 Hz, 6H). ESI-MS m/z calc. 133.11028, found 134.2 (M+1)⁺; Retention time: 1.56 minutes; LC method 1D.

### Step 8: 3-[[4-[(2R)-2-Amino-3-isopropoxy-propoxy]-6-(2,6-dimethylphenyl) pyrimidin-2-yl]sulfamoyl]benzoic acid

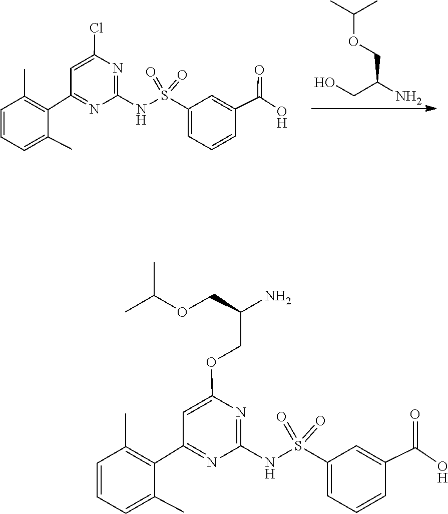

Sodium tert-butoxide (1.55 g, 16.128 mmol) was added in one portion to a solution of (2S)-2-amino-3-isopropoxy-propan-1-ol (449 mg, 3.2026 mmol) and 3-[[4-chloro-6-(2,6-dimethylphenyl)pyrimidin-2-yl]sulfamoyl]benzoic acid (1.11 g, 2.6564 mmol) in MeTHF (28 mL) and DMF (4 mL) at 15° C. The reaction was then stirred at room temperature for 1 h. More of 3-[[4-chloro-6-(2,6-dimethylphenyl)pyrimidin-2-yl]sulfamoyl]benzoic acid (370 mg, 0.8855 mmol) followed by sodium tert-butoxide (520 mg, 5.4108 mmol) were added and the reaction was stirred at room temperature for 1 h. The reaction was acidified with aqueous HCl 1N (50 mL) and the phases were separated. The aqueous layer was washed with EtOAc (3×50 mL). The combined organic layers were dried over sodium sulfate, filtered, and concentrated under reduced pressure. The residue was purified by reversed-phase chromatography (Column: C₁₈. Gradient: 5-100% methanol in water). The fractions containing the desired product were combined and evaporated to dryness. The residue was purified by reversed-phase chromatography (Column: C₁₈. Gradient: 5-60% MeCN in water containing 0.1% v/v HCl). The fractions containing the desired product were combined, evaporated to dryness and freeze-dried to afford 3-[[4-[(2R)-2-amino-3-isopropoxy-propoxy]-6-(2,6-dimethylphenyl)pyrimidin-2-yl]sulfamoyl]benzoic acid (hydrochloride salt) (60 mg, 3%) as a white solid. ESI-MS m/z calc. 514.1886, found 515.2 (M+1)⁺; Retention time: 1.33 minutes; LC method X.

### Step 9: 3-[[4-[(2R)-2-[[5-[Cyclobutyl(methyl)amino]pyrazin-2-yl]methylamino]-3-isopropoxy-propoxy]-6-(2,6-dimethylphenyl)pyrimidin-2-yl]sulfamoyl]benzoic acid

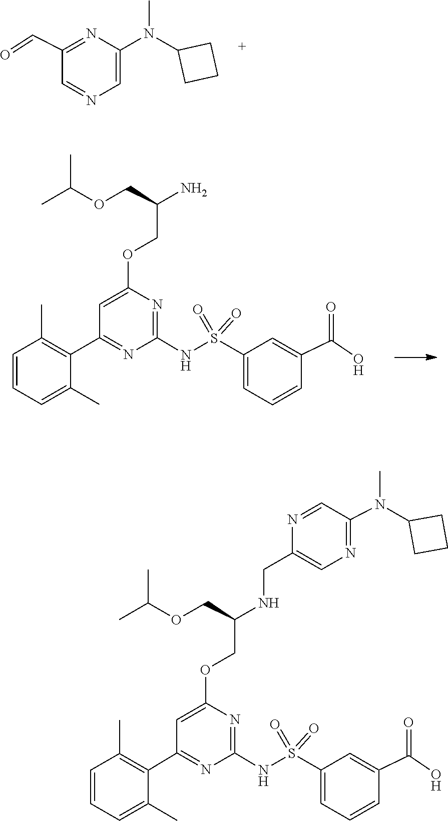 [SMILES: CN(c1cncc(C=O)n1)C1CCC1 ; Cc1cccc(C)c1-c1cc(OC[C@H](N)COC(C)C)nc(NS(=O)(=O)c2cccc(C(=O)O)c2)n1 ; Cc1cccc(C)c1-c1cc(OC[C@@H](COC(C)C)NCc2cnc(N(C)C3CCC3)cn2)nc(NS(=O)(=O)c2cccc(C(=O)O)c2)n1]

6-[Cyclobutyl(methyl)amino]pyrazine-2-carbaldehyde (24 mg, 0.1199 mmol) was added to a solution of 3-[[4-[(2R)-2-amino-3-isopropoxy-propoxy]-6-(2,6-dimethylphenyl)pyrimidin-2-yl]sulfamoyl]benzoic acid (60 mg, 0.1145 mmol) in anhydrous dichloromethane (4 mL) under nitrogen at 10° C. The solution was stirred at 10° C. for 15 minutes and sodium triacetoxyborohydride (72 mg, 0.3397 mmol) was added. The reaction was stirred at 10° C. for 1 h. An aqueous solution of 1N hydrochloric acid (10 mL) was added. The layers were separated, and the aqueous layer was extracted with dichloromethane (3×10 mL) and 2-methyltetrahydrofuran (3×15 mL). The combined organic layers were washed with brine, dried over magnesium sulfate, filtered and concentrated under reduced pressure to afford crude 3-[[4-[(2R)-2-[[5-[cyclobutyl(methyl)amino]pyrazin-2-yl]methylamino]-3-isopropoxy-propoxy]-6-(2,6-dimethylphenyl)pyrimidin-2-yl]sulfamoyl]benzoic acid (hydrochloride salt) (93 mg, 87%) as a yellow oil. ESI-MS m/z calc. 689.29956, found 690.2 (M+1)⁺; Retention time: 1.48 minutes; LC method X.

### Step 10: (11R)-12-[[6-[Cyclobutyl(methyl)amino]pyrazin-2-yl]methyl]-6-(2,6-dimethylphenyl)-11-(isopropoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1324)

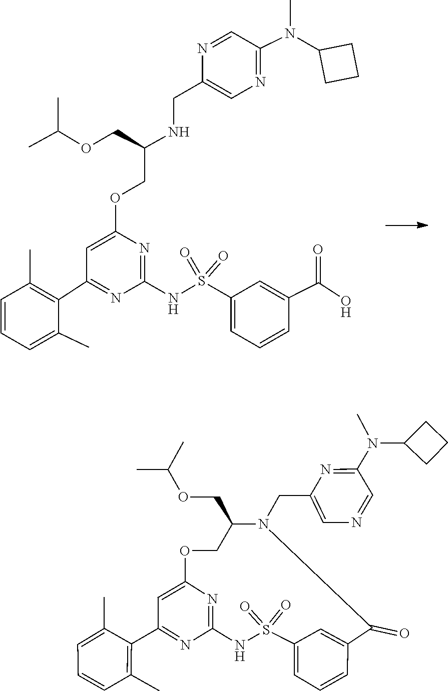 [SMILES: Cc1cccc(C)c1-c1cc(OC[C@@H](COC(C)C)NCc2cnc(N(C)C3CCC3)cn2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cncc(N(C)C3CCC3)n1)[C@H](COC(C)C)CO2]

Crude 3-[[4-[(2R)-2-[[5-[cyclobutyl(methyl)amino]pyrazin-2-yl]methylamino]-3-isopropoxy-propoxy]-6-(2,6-dimethylphenyl)pyrimidin-2-yl]sulfamoyl]benzoic acid (hydrochloride salt) (93 mg, 0.1088 mmol) was dissolved in anhydrous DMF (10 mL) under nitrogen and cooled to 0° C. N-Methylmorpholine (138.00 mg, 0.15 mL, 1.3644 mmol) was added to the solution and was stirred at 0° C. for 10 minutes. 2-Chloro-4,6-dimethoxy-1,3,5-triazine (48 mg, 0.2734 mmol) was then added and the reaction was stirred from 0° C. to room temperature for 3 days. The solvent was removed under reduced pressure and the crude yellow oil was purified by reverse-phase chromatography (Column: C₁₈. Gradient: 5 to 100% methanol in water containing 0.1% v/v of HCl). The fractions containing the desired product were combined and evaporated to dryness. The two isomers were separated by chiral SFC (Column Lux 5 m, Cellulose 1, 250×21.2 mm, 1.65 mg/injection volume, concentration 11 mg/mL, Column T=40° C., Flow rate 75 mL/min, 20% MeOH). The major isomer was then evaporated and freeze-dried. The product was further purified by reverse-phase chromatography (Column: C₁₈. Gradient: 5-100% methanol in basic water (buffer=pH 10, ammonium bicarbonate/ammonium hydroxide). The fractions containing the desired product were combined, evaporated to dryness, co-evaporated with water (3×15 mL) and freeze-dried to afford (11R)-12-[[6-[cyclobutyl(methyl)amino]pyrazin-2-yl]methyl]-6-(2,6-dimethylphenyl)-11-(isopropoxymethyl)-2,2-dioxo-9-oxa-2λ⁶-thia-3,5,12,19-tetrazatricyclo[12.3.1.14,8]nonadeca-1(18),4(19),5,7,14,16-hexaen-13-one (7.9 mg, 11%) as a beige solid. ¹H NMR (400 MHz, DMSO-d₆) δ 8.59 (s, 1H), 7.99 (s, 1H), 7.95-7.89 (m, 1H), 7.87 (s, 1H), 7.74-7.59 (m, 2H), 7.26 (t, J 7.7 Hz, 1H), 7.12 (d, J 7.6 Hz, 2H), 6.36 (br. s., 1H), 5.32 (dd, J 11.0, 4.4 Hz, 1H), 4.83-4.73 (m, 2H), 4.49 (d, J 16.1 Hz, 1H), 4.42 (t, J 11.5 Hz, 1H), 4.25-4.14 (m, 1H), 3.70 (dd, J 10.9, 8.9 Hz, 1H), 3.46 (dd, J 11.0, 3.4 Hz, 1H), 3.37-3.33 (m, 1H, overlapped with water), 3.05 (s, 3H), 2.24-2.12 (m, J 8.8, 8.8 Hz, 4H), 2.06 (br. s., 6H), 1.70-1.58 (m, 2H), 0.98 (d, J 5.9 Hz, 3H), 0.89 (d, J 6.1 Hz, 3H), (1H missing, labile proton). ESI-MS m/z calc. 671.289, found 672.2 (M+1)⁺; Retention time: 4.43 minutes; LC method Y.

### Example 165: Preparation of Compound 1325 and Compound 1326

### Step 1: 3-[[4-[(1-Amino-3,3-dimethyl-cyclopentyl)methoxy]-6-(2,6-dimethylphenyl)pyrimidin-2-yl]sulfamoyl]benzoic acid

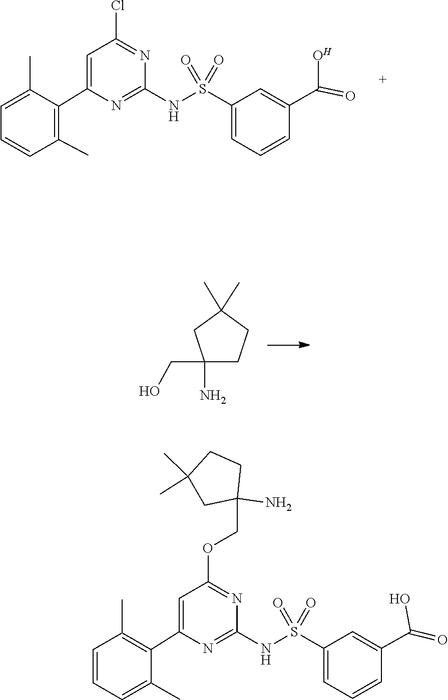

3-[[4-Chloro-6-(2,6-dimethylphenyl)pyrimidin-2-yl]sulfamoyl]benzoic acid (300 mg, 0.7179 mmol) was combined with (1-amino-3,3-dimethyl-cyclopentyl)methanol (hydrochloride salt) (114 mg, 0.7959 mmol) in THF (1.8 mL) and stirred for 5 minutes. Sodium tert-butoxide (350 mg, 3.642 mmol) was then added in one portion, and the reaction mixture was stirred vigorously at room temperature for 30 minutes. The temperature was increased to 45° C. for 30 minutes, then the reaction was cooled to room temperature and partitioned between 1M HCl and ethyl acetate. The layers were separated and the aqueous was extracted an additional 2× with ethyl acetate. The aqueous layer was then diluted with brine and extracted a further 2× ethyl aceate. The combined organics were washed with brine, dried over sodium sulfate and concentrated to give 3-[[4-[(1-amino-3,3-dimethyl-cyclopentyl)methoxy]-6-(2,6-dimethylphenyl)pyrimidin-2-yl]sulfamoyl]benzoic acid (hydrochloride salt) (433.2 mg, 97%), which was used without further purification. ESI-MS m/z calc. 524.20935, found 525.5 (M+1)⁺; Retention time: 0.48 minutes; LC method D.

### Step 2: 3-[[4-[[1-[[6-[Cyclobutyl(methyl)amino]pyrazin-2-yl]methylamino]-3,3-dimethyl-cyclopentyl]methoxy]-6-(2,6-dimethylphenyl)pyrimidin-2-yl]sulfamoyl]benzoic acid

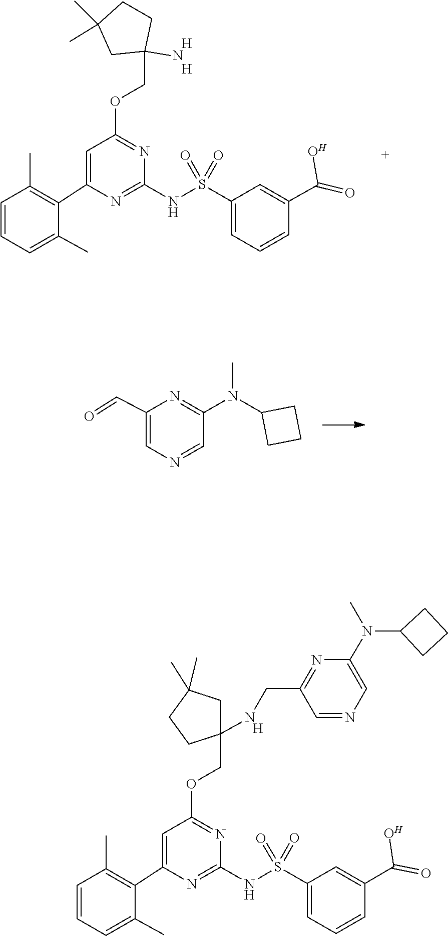

3-[[4-[(1-Amino-3,3-dimethyl-cyclopentyl)methoxy]-6-(2,6-dimethylphenyl)pyrimidin-2-yl]sulfamoyl]benzoic acid (hydrochloride salt) (60 mg, 0.09624 mmol) was combined with 6-[cyclobutyl(methyl)amino]pyrazine-2-carbaldehyde (20 mg, 0.1046 mmol) in dcm (0.4 mL), and stirred for 10 minutes at room temperature. Sodium triacetoxyborohydride (20 mg, 0.09437 mmol) was then added and the reaction was stirred for an additional 20 minutes. Additional sodium triacetoxyborohydride (61 mg, 0.2878 mmol) was added and stirring was continued for an additional 15 minutes. The reaction mixture was then quenched with 0.3 mL 3M HCl. The reaction mixture was diluted with 1:1 DMSO/methanol until it became homogeneous then filtered and purified by preparative HPLC (1-99% ACN in water HCl modifier, 15 minute run) to give 3-[[4-[[1-[[6-[cyclobutyl(methyl)amino]pyrazin-2-yl]methylamino]-3,3-dimethyl-cyclopentyl]methoxy]-6-(2,6-dimethylphenyl)pyrimidin-2-yl]sulfamoyl]benzoic acid (hydrochloride salt) (40 mg, 56%). ESI-MS m/z calc. 699.3203, found 700.9 (M+1)⁺; Retention time: 0.58 minutes; LC method D.

### Step 3: 12-[[6-[Cyclobutyl(methyl)amino]pyrazin-2-yl]methyl]-6-(2,6-dimethylphenyl)-1′,1′-dimethyl-2,2-dioxo-spiro[9-oxa-2λ⁶-thia-3,5,12,19-tetrazatricyclo[12.3.1.14,8]nonadeca-1(18),4(19),5,7,14,16-hexaene-11,3′-cyclopentane]-13-one

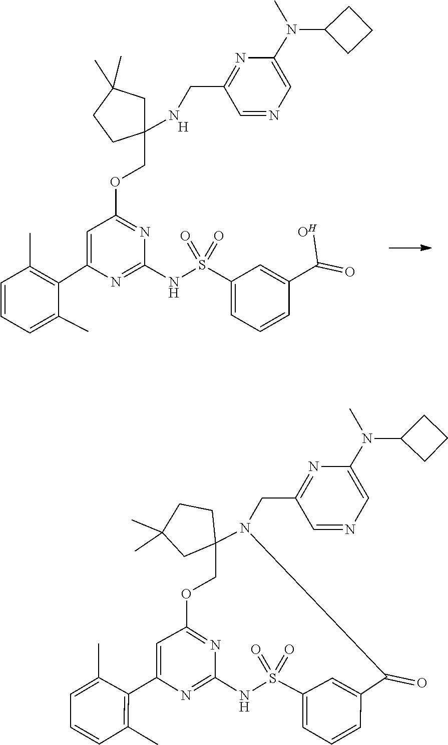

3-[[4-[[1-[[6-[Cyclobutyl(methyl)amino]pyrazin-2-yl]methylamino]-3,3-dimethyl-cyclopentyl]methoxy]-6-(2,6-dimethylphenyl)pyrimidin-2-yl]sulfamoyl]benzoic acid (hydrochloride salt) (40 mg, 0.05432 mmol) was combined with CDMT (11.5 mg, 0.06550 mmol) in DMF (3 mL), and N-methylmorpholine (40 μL, 0.3638 mmol) was added. The reaction was stirred for 48 hours at room temperature at which point an additional quantity of CDMT (6 mg, 0.03417 mmol) was added and the reaction was stirred for a further 72 hours. The reaction mixture was then partitioned between 5 mL 1M HCl and 5 mL ethyl acetate. The layers were separated and the aqueous was extracted an additional 3×ethyl acetate. The combined organics were washed with brine, dried over sodium sulfate, and concentrated. The resulting crude material was purified by flash chromatography on silica gel, eluting with 0-15% methanol in DCM in gradient to give 12-[[6-[cyclobutyl(methyl)amino]pyrazin-2-yl]methyl]-6-(2,6-dimethylphenyl)-1′,1′-dimethyl-2,2-dioxo-spiro[9-oxa-2?⁶-thia-3,5,12,19-tetrazatricyclo[12.3.1.14,8]nonadeca-1(18),4(19),5,7,14,16-hexaene-11,3′-cyclopentane]-13-one (13.4 mg, 36%). ESI-MS m/z calc. 681.30975, found 682.7 (M+1)⁺; Retention time: 0.76 minutes; LC method D.

### Step 4: 12-[[6-[cyclobutyl(methyl)amino]pyrazin-2-yl]methyl]-6-(2,6-dimethylphenyl)-1′,1′-dimethyl-2,2-dioxo-spiro[9-oxa-2λ⁶-thia-3,5,12,19-tetrazatricyclo[12.3.1.14,8]nonadeca-1(18),4(19),5,7,14,16-hexaene-11,3′-cyclopentane]-13-one, enantiomer 1 (Compound 1325) and 12-[[6-[cyclobutyl(methyl)amino]pyrazin-2-yl]methyl]-6-(2,6-dimethylphenyl)-1′,1′-dimethyl-2,2-dioxo-spiro[9-oxa-2λ⁶-thia-3,5,12,19-tetrazatricyclo[12.3.1.14,8]nonadeca-1(18),4(19),5,7,14,16-hexaene-11,3′-cyclopentane]-13-one, enantiomer 2 (Compound 1326)

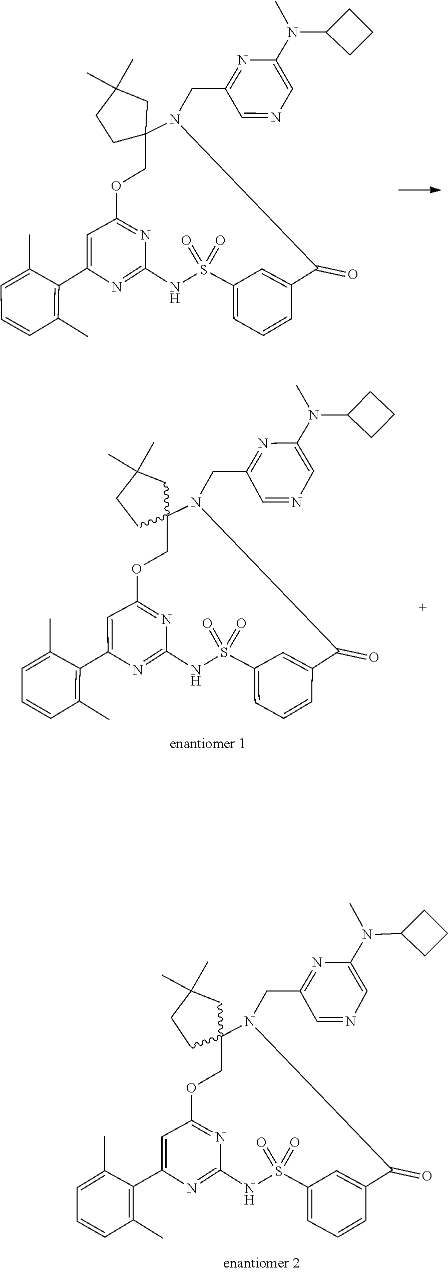

The enantiomers of 12-[[6-[cyclobutyl(methyl)amino]pyrazin-2-yl]methyl]-6-(2,6-dimethylphenyl)-1′,1′-dimethyl-2,2-dioxo-spiro[9-oxa-2?⁶-thia-3,5,12,19-tetrazatricyclo[12.3.1.14,8]nonadeca-1(18),4(19),5,7,14,16-hexaene-11,3′-cyclopentane]-13-one (13.4 mg, 0.01965 mmol) were separated by chiral SFC using a ChiralPak IG (21.2×250 mm, 5 μM) column at 40° C. Mobile phase was 56% MeOH (20 mM NH₃), 44% CO₂ at 70 mL/min flow. Concentration of the sample was 23 mg/mL in MeOH (no modifier), injection volume was 200 μL, outlet pressure at 237 bar, and detection wavelength 210 nm, to give Enantiomer 1 (first eluting SFC), 12-[[6-[cyclobutyl(methyl)amino]pyrazin-2-yl]methyl]-6-(2,6-dimethylphenyl)-1′,1′-dimethyl-2,2-dioxo-spiro[9-oxa-2?⁶-thia-3,5,12,19-tetrazatricyclo[12.3.1.14,8]nonadeca-1(18),4(19),5,7,14,16-hexaene-11,3′-cyclopentane]-13-one (ammonium salt) (3.3 mg, 24%) ESI-MS m/z calc. 681.30975, found 682.8 (M+1)⁺; Retention time: 1.9 minutes and Enantiomer 2 (second eluting SFC) 12-[[6-[cyclobutyl(methyl)amino]pyrazin-2-yl]methyl]-6-(2,6-dimethylphenyl)-1′,1′-dimethyl-2,2-dioxo-spiro[9-oxa-2?⁶-thia-3,5,12,19-tetrazatricyclo[12.3.1.14,8]nonadeca-1(18),4(19),5,7,14,16-hexaene-11,3′-cyclopentane]-13-one (ammonium salt) (3.1 mg, 22%) ESI-MS m/z calc. 681.30975, found 682.8 (M+1)⁺; Retention time: 1.9 minutes; LC method A.

### Example 166: Preparation of Compound 1327 and Compound 1328

### Step 1: Ethyl 3-hydroxy-3-methyl-butanoate

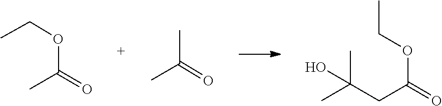

Ethyl acetate (5.0512 g, 5.6 mL, 57.332 mmol) was added dropwise to a solution of (bis(trimethylsilyl)amino)lithium (in THF) (39 mL of 1.5 M, 58.500 mmol) in THF (56 mL) at −78° C. The reaction mixture was stirred at this temperature for 30 min. Acetone (3.9550 g, 5 mL, 68.097 mmol) was added and the reaction mixture was left stirring 10 min. HCl (2 M, 35 ml) was added to the reaction mixture which was then left to warm up to room temperature. The reaction mixture was extracted with ethyl acetate (2×100 mL). The combined organic phases were washed with saturated aqueous sodium bicarbonate (50 mL), dried with sodium sulfate, filtered and concentrated under reduced pressure to afford ethyl 3-hydroxy-3-methyl-butanoate (7.84 g, 89%) as a clear yellow oil ¹H NMR (400 MHz, CDCl₃) δ 4.18 (q, J 7.1 Hz, 2H), 3.59 (s, 1H), 2.48 (s, 2H), 1.31-1.26 (m, 9H). ESI-MS m/z calc. 146.0943, found 169.2 (M+23)⁺; Retention time: 1.3 minutes; LC method X.

### Step 2: Ethyl 3-fluoro-3-methyl-butanoate

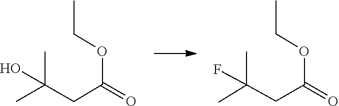

Deoxo-Fluor (solution in toluene) (26 g, 50% w/w, 58.759 mmol) was added to a solution of ethyl 3-hydroxy-3-methyl-butanoate (7.5 g, 48.740 mmol) in DCM (125 mL) at −78° C. The reaction was then left to warm-up to room temperature and stirred for 4 h. The reaction mixture was quenched with aqueous sodium bicarbonate (200 mL). The aqueous phase was extracted with DCM (2×100 mL) and the combined organic phases were washed with saturated aqueous ammonium chloride (100 mL), dried with magnesium sulfate, filtered and concentrated under reduced pressure to provide crude ethyl 3-fluoro-3-methyl-butanoate (4.8 g, 53%) as a clear oil ¹H NMR (400 MHz, CDCl₃) δ 4.16 (q, J 7.2 Hz, 2H), 2.66 (d, J 16.1 Hz, 2H), 1.49 (d, J 21.8 Hz, 6H), 1.28 (t, J 7.2 Hz, 3H). Which was used directly in the next step without further purification.

### Step 3: 3-Fluoro-3-methyl-butanal

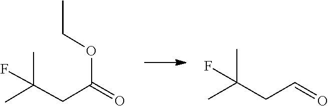

DIBAL (in toluene) (8.7 mL of 1 M, 8.7000 mmol) was slowly added to a solution of ethyl 3-fluoro-3-methyl-butanoate (1 g, 5.3990 mmol) in DCM (10 mL) at −78° C. The reaction mixture was left to stir at this temperature for 1 h. The reaction mixture was quenched with concentrated aqueous ammonium chloride (20 mL) and 1N HCl (5 mL). The reaction mixture was left to warm-up to room temperature and left stirring for 30 min. DCM (100 mL) was added to the mixture and shaken. The aqueous phase was separated and washed with more DCM (25 mL). The combined organic phases were dried with sodium sulfate overnight then filtered to provide a ˜0.3% solution of 3-fluoro-3-methyl-butanal (185 g, 99%) as a clear solution. This solution was used directly in the next step.

### Step 4: Methyl 2-(tert-butoxycarbonylamino)-5-fluoro-5-methyl-hex-2-enoate

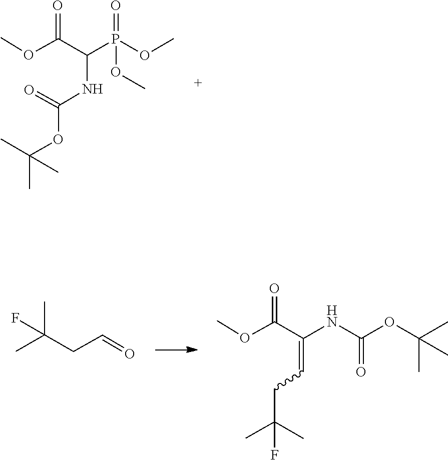

To a stirred solution of 3-fluoro-3-methyl-butanal (3% in DCM) (185 g, 5.3302 mmol) at 0° C. was added methyl 2-(tert-butoxycarbonylamino)-2-dimethoxyphosphoryl-acetate (500 mg, 1.6821 mmol) followed by 1,1,3,3-tetramethylguanidine (580 mg, 5.0357 mmol). The reaction mixture was stirred at 0° C. for 1 hour and then at room temperature for 18 hours. Water (100 mL) and DCM (100 mL) were added and the mixture was extracted with DCM (3×100 mL). The combined organic layers were washed with brine (50 mL), dried over sodium sulfate, filtered and concentrated under reduced pressure. The resulting residue was purified by flash-chromatography on a 40 g silica gel cartridge, using a gradient of 0 to 40% of EtOAc in heptanes to provide methyl 2-(tert-butoxycarbonylamino)-5-fluoro-5-methyl-hex-2-enoate (383 mg, 83%) as a white solid ¹H NMR (400 MHz, CDCl₃) δ 6.62 (t, J 7.3 Hz, 1H), 6.11 (br. s., 1H), 3.80 (s, 3H), 2.54 (dd, J 20.3, 7.6 Hz, 2H), 1.47 (s, 9H), 1.40 (d, J 21.5 Hz, 6H). 19F NMR (377 MHz, CDCl₃) δ −138.20 (br. s., 1F). ESI-MS m/z calc. 275.1533, found 298.2 (M+23)⁺; Retention time: 1.73 minutes; LC method X.

### Step 5: Methyl 2-(tert-butoxycarbonylamino)-5-fluoro-5-methyl-hexanoate

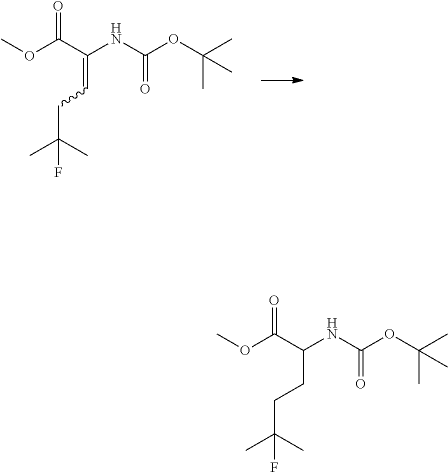 [SMILES: COC(=O)C(=CCC(C)(C)F)NC(=O)OC(C)(C)C ; COC(=O)C(CCC(C)(C)F)NC(=O)OC(C)(C)C]

Palladium (on carbon) (400 mg, 0.1879 mmol) was added to a solution of methyl 2-(tert-butoxycarbonylamino)-5-fluoro-5-methyl-hex-2-enoate (380 mg, 1.3802 mmol) in Methanol (4 mL) and hydrogen was injected into the suspension with a hydrogen balloon equipped with a thin needle on a continuous manner for 30 min. The crude mixture was filtered with a syringe filter and concentrated under reduced pressure. The resulting residue was purified by reverse phase chromatography on a C₁₈ column using 5 to 100% acetonitrile in acid water (with 0.1% formic acid) to provide methyl 2-(tert-butoxycarbonylamino)-5-fluoro-5-methyl-hexanoate (250 mg, 65%) as a clear oil ¹H NMR (400 MHz, CDCl₃) δ 5.03 (d, J 7.1 Hz, 1H), 4.38-4.26 (m, 1H), 3.76 (s, 3H), 2.04-1.89 (m, 1H), 1.80-1.71 (m, 1H), 1.69-1.57 (m, 2H), 1.45 (s, 9H), 1.34 (d, J 21.5 Hz, 6H). ESI-MS m/z calc. 277.1689, found 300.2 (M+23)⁺; Retention time: 1.76 minutes; LC method X.

### Step 6: tert-Butyl N-[4-fluoro-1-(hydroxymethyl)-4-methyl-pentyl]carbamate

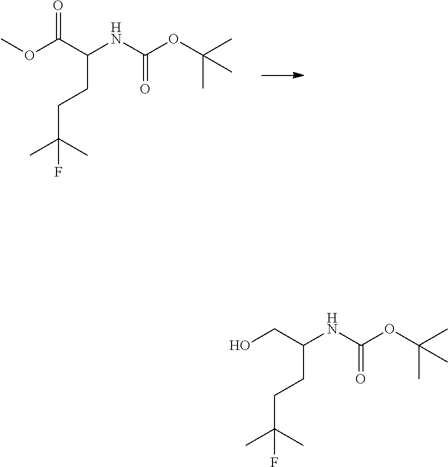 [SMILES: COC(=O)C(CCC(C)(C)F)NC(=O)OC(C)(C)C ; CC(C)(F)CCC(CO)NC(=O)OC(C)(C)C]

To a solution of methyl 2-(tert-butoxycarbonylamino)-5-fluoro-5-methyl-hexanoate (230 mg, 0.8293 mmol) in ethanol (6 mL) at 0° C. was added lithium borohydride (340 mg, 15.608 mmol). This reaction mixture was stirred 1 h at this temperature and then left to warm-up to room temperature stirred for another 30 min, then water (20 mL) was added to the reaction mixture which was then left stirring overnight at room temperature. The reaction mixture was transferred into a cold aqueous 0.1N HCl (40 mL) solution and this mixture was extracted with DCM (3×50 mL). The combined organic phases were dried with sodium sulfate, filtered and concentrated under reduced pressure to provide tert-butyl N-[4-fluoro-1-(hydroxymethyl)-4-methyl-pentyl]carbamate (240 mg, 99%) as a clear oil ¹H NMR (400 MHz, CDCl₃) δ 4.66 (br. s., 1H), 3.78-3.53 (m, 3H), 2.32 (br. s., 1H), 1.82-1.58 (m, 4H), 1.46 (s, 9H), 1.36 (d, J 21.8 Hz, 6H). 19F NMR (377 MHz, CDCl₃) δ −139.16 (br. s., 1F). ESI-MS m/z calc. 249.174, found 272.2 (M+23)⁺; Retention time: 1.63 minutes; LC method X.

### Step 7: 2-Amino-5-fluoro-5-methyl-hexan-1-ol

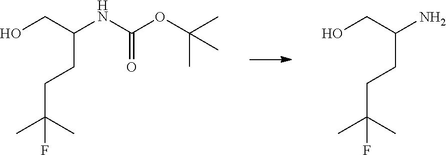

Hydrogen chloride (in dioxane) (2 mL of 4 M, 8.0000 mmol) was added to a solution of tert-butyl N-[4-fluoro-1-(hydroxymethyl)-4-methyl-pentyl]carbamate (240 mg, 0.8182 mmol) in DCM (2 mL) and the reaction mixture was left stirring at room temperature for 1 h. The reaction mixture was concentrated under reduced pressure and the resulting residue was dissolved in pure water and concentrated under reduced pressure the resulting residue was then redissolved in water and lyophilized to provide 2-amino-5-fluoro-5-methyl-hexan-1-ol (hydrochloride salt) (138 mg, 86%) white solid ¹H NMR (400 MHz, DMSO-d₆) δ 7.95 (br. s., 3H), 5.30 (t, J 5.0 Hz, 1H), 3.59 (dt, J 11.5, 4.4 Hz, 1H), 3.45 (dt, J 11.4, 5.7 Hz, 1H), 3.10-2.99 (m, 1H), 1.71-1.57 (m, 4H), 1.30 (d, J 22.0 Hz, 6H). 19F NMR (377 MHz, DMSO-d₆) δ −136.12 (nonu, J 20.4 Hz, 1F). ESI-MS m/z calc. 149.1216, found 150.2 (M+1)⁺; Retention time: 0.29 minutes; LC method X.

### Step 8: 3-[[4-(2-Amino-5-fluoro-5-methyl-hexoxy)-6-(2,6-dimethylphenyl)pyrimidin-2-yl]sulfamoyl]benzoic acid

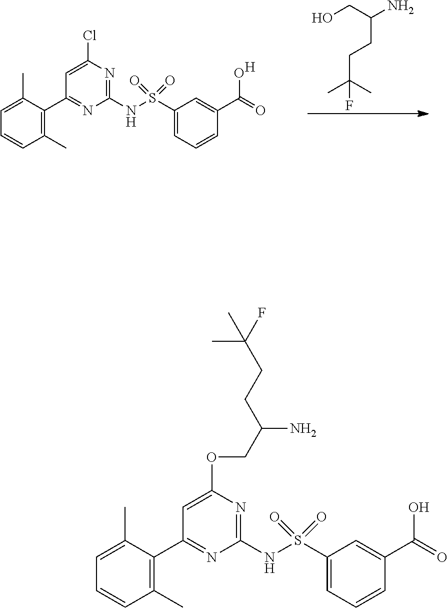

A flame-dried flask under nitrogen atmosphere was charged with 3-[[4-chloro-6-(2,6-dimethylphenyl)pyrimidin-2-yl]sulfamoyl]benzoic acid (325 mg, 0.7778 mmol), 2-amino-5-fluoro-5-methyl-hexan-1-ol (hydrochloride salt) (134 mg, 0.6856 mmol), 2-MeTHF (15 mL) and anhydrous DMF (1.5 mL). The reaction mixture was cooled down to 0° C. then sodium tert-butoxide (375 mg, 3.9020 mmol) was added. The reaction was stirred for 5 minutes at 0° C. then allowed to reach room temperature and stirred at room temperature for 45 minutes. The reaction was then cooled down to 0° C. then diluted with 2-methyltetrahydrofuran (150 mL) and quenched by the addition of an aqueous solution of 1N hydrochloric acid (150 mL). The layers were separated, and the aqueous layer was extracted with 2-methyltetrahydrofuran (2×150 mL). The combined organic layers were dried over sodium sulfate, filtered and concentrated under reduced pressure and the resulting residue was purified by reverse phase chromatography on a C₁₈ using a 50 g Gold column and eluting with a 5 to 100% gradient of acetonitrile in acidic water (containing 0.1% v/v of hydrochloric acid). The desired fractions were concentrated under reduced pressure, then freeze-dried to afford 3-[[4-(2-amino-5-fluoro-5-methyl-hexoxy)-6-(2,6-dimethylphenyl)pyrimidin-2-yl]sulfamoyl]benzoic acid (hydrochloride salt) (305 mg, 64%) as a white fluffy solid ¹H NMR (400 MHz, DMSO-d₆) δ 13.32 (br. s., 1H), 12.60-11.86 (m, 1H), 8.45 (t, J 1.6 Hz, 1H), 8.40-8.03 (m, 5H), 7.69 (t, J 7.7 Hz, 1H), 7.32-7.20 (m, 1H), 7.18-7.06 (m, 2H), 6.32 (br. s., 1H), 4.49-4.34 (m, 1H), 4.28 (dd, J 11.9, 6.2 Hz, 1H), 3.61-3.50 (m, 1H), 2.00 (br. s., 6H), 1.83-1.61 (m, 4H), 1.38-1.27 (m, 6H). 19F NMR (377 MHz, DMSO-d₆) δ −136.46 (s, 1F). ESI-MS m/z calc. 530.1999, found 531.1 (M+1)⁺; Retention time: 2.42 minutes; LC method Y.

### Step 9: Methyl 6-[(2R)-2-methyl-1-piperidyl]pyrazine-2-carboxylate

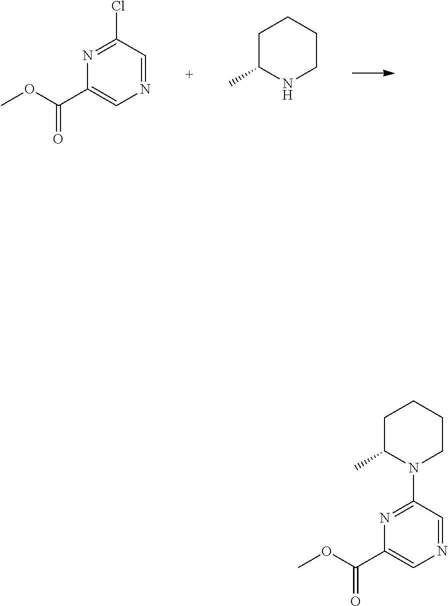

To a stirring solution of methyl 6-chloropyrazine-2-carboxylate (18.5 g, 105.06 mmol) and (2R)-2-methylpiperidine (12.98 g, 126.95 mmol) in anhydrous DMSO (250 mL) was added anhydrous sodium carbonate (22.3 g, 210.40 mmol). The resulting black mixture was stirred at 120° C. overnight. After cooling to room temperature, cold water (500 mL) was added, and the resulting solution was extracted with EtOAc (3×300 mL). The combined organic solutions were washed with water (500 mL), followed by brine (500 mL), dried over anhydrous sodium sulfate, and filtered. The solvent was removed by rotary evaporation and the residue was dried in vacuo yielding methyl 6-[(2R)-2-methyl-1-piperidyl]pyrazine-2-carboxylate (7 g, 28%) as an amber oil. The crude was used in the next step without further purification. ESI-MS m/z calc. 235.13208, found 236.3 (M+1)⁺; Retention time: 2.6 minutes; LC method T.

### Step 10:[6-[(2R)-2-Methyl-1-piperidyl]pyrazin-2-yl]methanol

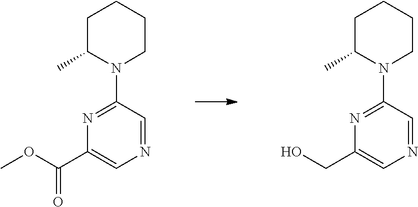 [SMILES: COC(=O)c1cncc(N2CCCC[C@H]2C)n1 ; C[C@@H]1CCCCN1c1cncc(CO)n1]

A solution of methyl 6-[(2R)-2-methyl-1-piperidyl]pyrazine-2-carboxylate (7 g, 29.752 mmol) in MeOH (200 mL) under nitrogen was cooled to 0° C. in an ice bath. NaBH4 (11.3 g, 298.68 mmol) was then added in portions at the same temperature over 5 min. The reaction mixture was stirred for 2 h at 0° C. then at room temperature overnight. The reaction was quenched with water (100 mL), saturated with sodium chloride, and extracted with DCM (3×100 mL). The combined organic solutions were dried over anhydrous sodium sulfate and filtered. The solvent was removed by rotary evaporation and the residue was dried in vacuo yielding [6-[(2R)-2-methyl-1-piperidyl]pyrazin-2-yl]methanol (5.82 g, 90%) as an orange oil. The crude was used in the next step without further purification. ESI-MS m/z calc. 207.13716, found 208.1 (M+1)⁺; Retention time: 1.57 minutes; LC method T.

### Step 11: 6-[(2R)-2-Methyl-1-piperidyl]pyrazine-2-carbaldehyde

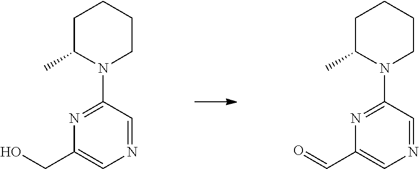

A solution of [6-[(2R)-2-methyl-1-piperidyl]pyrazin-2-yl]methanol (5.82 g, 26.675 mmol) in anhydrous DCM (200 mL) under nitrogen was cooled to 0° C. using an ice bath. DMP (17 g, 40.081 mmol) was then added in portions over 5 min. The resulting amber solution was warmed up to room temperature and stirred overnight. The reaction mixture was added with saturated aqueous sodium bicarbonate solution (300 mL) and stirred for 15 min. The organic layer was separated and washed further with saturated aqueous sodium bicarbonate solution (2×300 mL). The organic layer was dried over anhydrous sodium sulfate and filtered. The solvent was removed by rotary evaporation and the crude aldehyde was purified by silica flash chromatography (330 g, dry loaded, eluting from 0 to 30% EtOAc in hexanes over a 65 min gradient). The fractions were combined and concentrated under reduced pressure and the residue was further dried in vacuo yielding 6-[(2R)-2-methyl-1-piperidyl]pyrazine-2-carbaldehyde (1.2085 g, 20%) as an orange liquid. ¹H NMR (500 MHz, DMSO-d₆) δ 9.88 (s, 1H), 8.53 (s, 1H), 8.23 (s, 1H), 4.83-4.72 (m, 1H), 4.35-4.28 (m, 1H), 2.99 (td, J 13.1, 2.9 Hz, 1H), 1.79-1.73 (m, 1H), 1.73-1.64 (m, 2H), 1.64-1.56 (m, 2H), 1.51-1.37 (m, 1H), 1.17 (d, J 6.8 Hz, 3H). ESI-MS m/z calc. 205.1215, found 206.2 (M+1)⁺; Retention time: 2.26 minutes; LC method W.

### Step 12: 3-[[4-(2,6-Dimethylphenyl)-6-[5-fluoro-5-methyl-2-[[6-[(2R)-2-methyl-1-piperidyl]pyrazin-2-yl]methylamino]hexoxy]pyrimidin-2-yl]sulfamoyl]benzoic acid

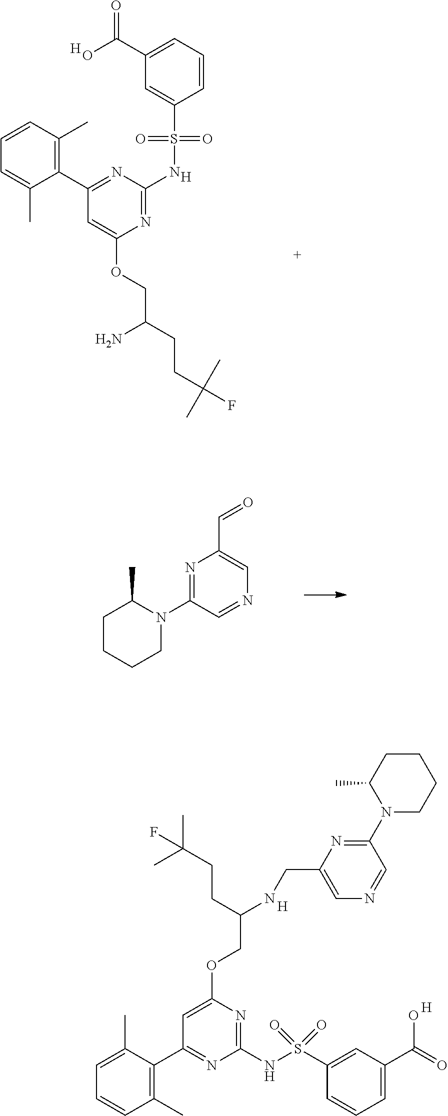

3-[[4-(2-Amino-5-fluoro-5-methyl-hexoxy)-6-(2,6-dimethylphenyl)pyrimidin-2-yl]sulfamoyl]benzoic acid (hydrochloride salt) (60 mg, 0.1058 mmol) was combined with 6-[(2R)-2-methyl-1-piperidyl]pyrazine-2-carbaldehyde (approximately 23.89 mg, 0.1164 mmol) in DCM (0.3 mL). The reaction mixture was stirred for 15 minutes at room temperature and sodium triacetoxyborohydride (approximately 22.42 mg, 0.1058 mmol) was added. After a further 15 minutes an additional portion of sodium triacetoxyborohydride (approximately 67.27 mg, 0.3174 mmol) was added. After an additional 45 minutes the reaction mixture was quenched with 0.2 mL 3M HCl and diluted with DMSO and methanol until the reaction mixture became homogeneous. The reaction mixture was then filtered and purified by preparative HPLC (1-70% ACN in water, HCl monitor, 15 minute run) to give 3-[[4-(2,6-dimethylphenyl)-6-[5-fluoro-5-methyl-2-[[6-[(2R)-2-methyl-1-piperidyl]pyrazin-2-yl]methylamino]hexoxy]pyrimidin-2-yl]sulfamoyl]benzoic acid (hydrochloride salt) (45 mg, 56%) upon drying. ESI-MS m/z calc. 719.32654, found 720.8 (M+1)⁺; Retention time: 0.58 minutes; LC method D.

### Step 13: 6-(2,6-Dimethylphenyl)-11-(3-fluoro-3-methyl-butyl)-12-[[6-[(2R)-2-methyl-1-piperidyl]pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one

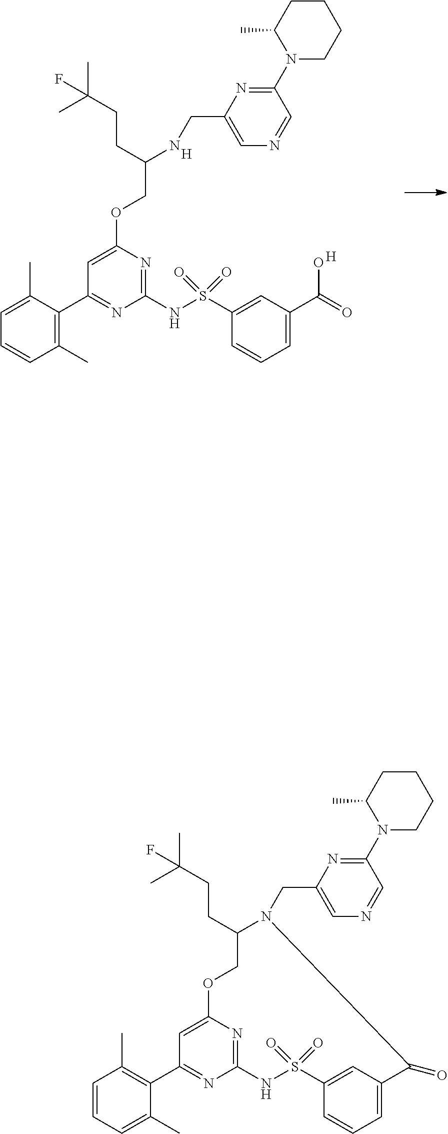

3-[[4-(2,6-Dimethylphenyl)-6-[5-fluoro-5-methyl-2-[[6-[(2R)-2-methyl-1-piperidyl]pyrazin-2-yl]methylamino]hexoxy]pyrimidin-2-yl]sulfamoyl]benzoic acid (hydrochloride salt) (45 mg, 0.05950 mmol) was combined with CDMT (13.5 mg, 0.07689 mmol) in DMF (3 mL) in a screwcap vial. N-methylmorpholine (40 μL, 0.3638 mmol) was added and the reaction was stirred for 16 hours at room temperature. The reaction mixture was then partitioned between 5 mL HCl and 10 mL ethyl acetate. The layers were separated and the aqueous was extracted an additional 2× with ethyl acetate. The combined organics were washed with brine, dried over sodium sulfate and concentrated. The resulting crude mixture was purified by chromatography on silica gel eluting with a 0-15% methanol in DCM gradient to give 6-(2,6-dimethylphenyl)-11-(3-fluoro-3-methyl-butyl)-12-[[6-[(2R)-2-methyl-1-piperidyl]pyrazin-2-yl]methyl]-2,2-dioxo-9-oxa-2?⁶-thia-3,5,12,19-tetrazatricyclo[12.3.1.14,8]nonadeca-1(18),4(19),5,7,14,16-hexaen-13-one (22.5 mg, 54%). ESI-MS m/z calc. 701.316, found 702.8 (M+1)⁺; Retention time: 0.71 minutes; LC method D.

### Step 14: 6-(2,6-Dimethylphenyl)-11-(3-fluoro-3-methyl-butyl)-12-[[6-[(2R)-2-methyl-1-piperidyl]pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 1327) and 6-(2,6-dimethylphenyl)-11-(3-fluoro-3-methyl-butyl)-12-[[6-[(2R)-2-methyl-1-piperidyl]pyrazin-2-yl]methyl]-2,2-dioxo-9-oxa-2)⁶-thia-3,5,12,19-tetrazatricyclo[12.3.1.14,8]nonadeca-1(18),4(19),5,7,14,16-hexaen-13-one, diastereomer 2 (Compound 1328)

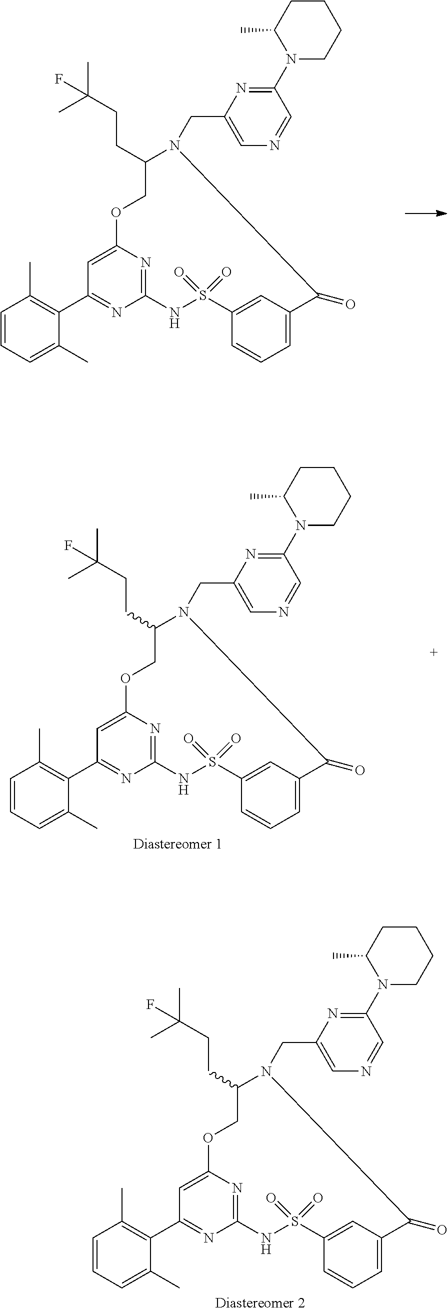 [SMILES: Cc1cccc(C)c1-c1cc2nc(n1)NS(=O)(=O)c1cccc(c1)C(=O)N(Cc1cncc(N3CCCC[C@H]3C)n1)C(CCC(C)(C)F)CO2 ; Cc1cccc(C)c1-c1cc2nc(n1)NS(=O)(=O)c1cccc(c1)C(=O)N(Cc1cncc(N3CCCC[C@H]3C)n1)C(CCC(C)(C)F)CO2 ; Cc1cccc(C)c1-c1cc2nc(n1)NS(=O)(=O)c1cccc(c1)C(=O)N(Cc1cncc(N3CCCC[C@H]3C)n1)C(CCC(C)(C)F)CO2]

The diastereomers of 6-(2,6-dimethylphenyl)-11-(3-fluoro-3-methyl-butyl)-12-[[6-[(2R)-2-methyl-1-piperidyl]pyrazin-2-yl]methyl]-2,2-dioxo-9-oxa-2⁶-thia-3,5,12,19-tetrazatricyclo[12.3.1.14,8]nonadeca-1(18),4(19),5,7,14,16-hexaen-13-one (22 mg, 0.03135 mmol) were separated by chiral SFC using a Phenomenex LUX-4 (21.2×250 mm, 5 PM) column at 50° C. Mobile phase was 36% MeOH (20 mM NH₃), 64% CO₂ at 70 mL/min flow. Concentration of the sample was 20 mg/mL in 2:1 MeOH/DMSO, injection volume was 200 μL, outlet pressure at 216 bar, and detection wavelength 210 nm. Each diastereomer was collected separately and the resulting products were each further purified by preparative HPLC (1-99% ACN in water, HCl modifier, 15 minute run) to give Diastereomer 1 (first eluting SFC), 6-(2,6-dimethylphenyl)-11-(3-fluoro-3-methyl-butyl)-12-[[6-[(2R)-2-methyl-1-piperidyl]pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (2.9 mg, 13%) ESI-MS m/z calc. 701.316, found 702.8 (M+1)⁺; Retention time: 1.77 minutes and Diastereomer 2 (Second eluting SFC) 6-(2,6-dimethylphenyl)-11-(3-fluoro-3-methyl-butyl)-12-[[6-[(2R)-2-methyl-1-piperidyl]pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (2.8 mg, 13%) ESI-MS m/z calc. 701.316, found 702.8 (M+1)⁺; Retention time: 1.77 minutes; LC method A.

### Example 167: Preparation of Compound 1329 and Compound 1330

### Step 1: Diethyl 2-(1,1-dimethylpropyl)propanedioate

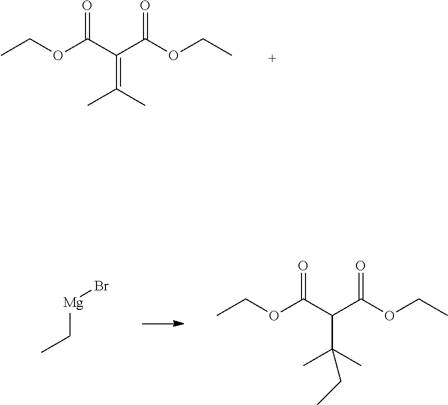

To a solution of diethyl isopropylidenemalonate (2 g, 9.9884 mmol) in THF (60 mL) was added CuI (2.85 g, 14.965 mmol). After stirring for 30 minutes at 0° C., ethylmagnesium bromide solution in THF (30 mL of 1 M, 30.000 mmol) was added dropwise and the mixture was stirred at 0° C. for 3 hours. The mixture was quenched with 1N HCl (50 mL) and extracted with ethyl acetate (2×60 mL). The combined organic phases was washed with brine (2×50 mL), dried over anhydrous sodium sulfate, filtered and concentrated under reduced pressure to afford diethyl 2-(1,1-dimethylpropyl)propanedioate (2.4 g, 99%) as a brownish oil. ¹H NMR (400 MHz, CDCl₃) δ 4.18 (q, J 7.2 Hz, 4H), 3.33 (s, 1H), 1.48 (q, J 7.4 Hz, 2H), 1.27 (t, J 7.2 Hz, 6H), 1.09 (s, 6H), 0.87 (t, J 7.6 Hz, 3H). ESI-MS m/z calc. 230.15181, found 231.2 (M+1)⁺; Retention time: 1.98 minutes; LC method X.

### Step 2: 3,3-Dimethylpentanoic acid

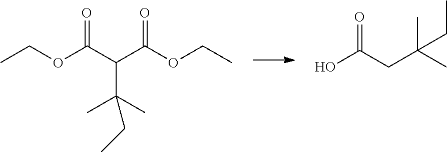

To a solution of diethyl 2-(1,1-dimethylpropyl)propanedioate (2.4 g, 9.9001 mmol) in DMSO (50 mL) and water (10 mL) was added lithium hydroxide hydrate (2.1 g, 50.043 mmol) and the mixture was stirred at 120° C. for 22 hours. The mixture was acidified to pH 2-3 with 1N hydrochloric acid and extracted with ethyl acetate (2×40 mL). The combined organic layers were washed with brine (3×30 mL) and water (30 mL), dried over sodium sulfate, filtered, and concentrated under reduced pressure to give 3,3-dimethylpentanoic acid (1.23 g, 91%) as a yellow oil. ¹H NMR (400 MHz, CDCl₃) δ 2.23 (s, 2H), 1.39 (q, J 7.4 Hz, 2H), 1.02 (s, 6H), 0.87 (t, J 7.5 Hz, 3H); one labile proton missing. ESI-MS m/z calc. 130.09938, found 131.2 (M+1)⁺; Retention time: 1.6 minutes; LC method X.

### Step 3: N-Methoxy-N,3,3-trimethyl-pentanamide

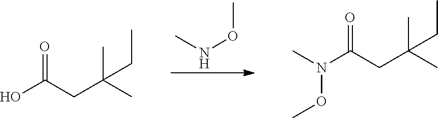

To a solution of 3,3-dimethylpentanoic acid (1.23 g, 8.9757 mmol) in DMF (28 mL) was added at 15-20° C. (water bath)N-methoxymethanamine (hydrochloride salt) (1.1 g, 11.277 mmol), DIPEA (3.6358 g, 4.9 mL, 28.132 mmol) and then HATU (5.39 g, 14.176 mmol). The mixture was stirred for 16 hours at room temperature. The mixture was diluted with water (50 mL) and extracted with MTBE (2×75 mL). The combined organic phases were washed with brine (5×50 mL), dried over anhydrous sodium sulfate, filtered and concentrated under reduced pressure. Purification by silica gel chromatography (Column: 100 g. Gradient: 0-20% ethyl acetate in heptanes) afforded N-methoxy-N,3,3-trimethyl-pentanamide (1.19 g, 76%) as a colorless oil. ¹H NMR (400 MHz, CDCl₃) δ 3.67 (s, 3H), 3.18 (s, 3H), 2.31 (s, 2H), 1.40 (q, J 7.4 Hz, 2H), 1.00 (s, 6H), 0.86 (t, J=7.5 Hz, 3H). ESI-MS m/z calc. 173.14159, found 174.2 (M+1)⁺; Retention time: 1.72 minutes; LC method X.

### Step 4: 3,3-Dimethylpentanal

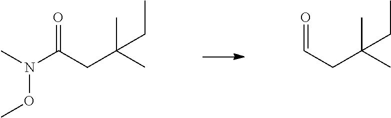

To a solution of N-methoxy-N,3,3-trimethyl-pentanamide (1.12 g, 6.3999 mmol) in THF (20 mL) was added lithium aluminum hydride (736 mg, 19.392 mmol) at 0° C. The mixture was stirred at 0° C. for 1 hour. The reaction mixture was slowly quenched with water (40 mL) and extracted with MTBE (2×30 mL). The combined organic layers were washed with brine (3×45 mL), dried over sodium sulfate and filtered to give crude 3,3-dimethylpentanal (730.77 mg, 100%) as a colorless solution in MTBE/THF.

### Step 5: 2-(Benzylamino)-4,4-dimethyl-hexanenitrile

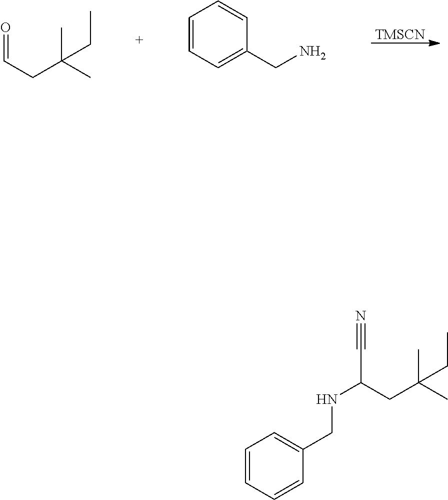

A three-neck round bottom flask equipped with a 6N NaOH trap, was charged with 3,3-dimethylpentanal (730.77 mg, 6.3999 mmol) as a 3:1 MTBE/THF solution (˜80 mL) and benzylamine (765.18 mg, 0.78 mL, 7.1410 mmol). To the mixture was slowly added at 0° C. acetic acid (411.84 mg, 0.39 mL, 6.8581 mmol) and then trimethylsilylcyanide (642.33 mg, 0.81 mL, 6.4747 mmol). The mixture was warmed to room temperature and stirred for 18 hours. The mixture was diluted with water (50 mL) and extracted with ethyl acetate (2×30 mL). The combined organic layers were washed with brine (2×40 mL), dried over anhydrous sodium sulfate, filtered, and concentrated under reduced pressure. Purification by chromatography on silica gel (Column: 120 g. Gradient: 0-10% methanol in dichloromethane) afforded 2-(benzylamino)-4,4-dimethyl-hexanenitrile (761 mg, 49%) as a light yellow oil. ¹H NMR (400 MHz, CDCl₃) δ 7.40-7.28 (m, 5H), 4.08 (d, J 12.7 Hz, 1H), 3.83 (d, J 13.0 Hz, 1H), 3.51 (dd, J 7.6, 5.6 Hz, 1H), 1.83 (dd, J 14.2, 7.6 Hz, 1H), 1.66 (dd, J 14.1, 5.5 Hz, 1H), 1.34-1.25 (m, 3H), 0.94 (s, 6H), 0.84 (t, J 7.6 Hz, 3H). ESI-MS m/z calc. 230.1783, found 231.4 (M+1)⁺; Retention time: 1.95 minutes; LC method X.

### Step 6: 2-(Benzylamino)-4,4-dimethyl-hexanoic acid

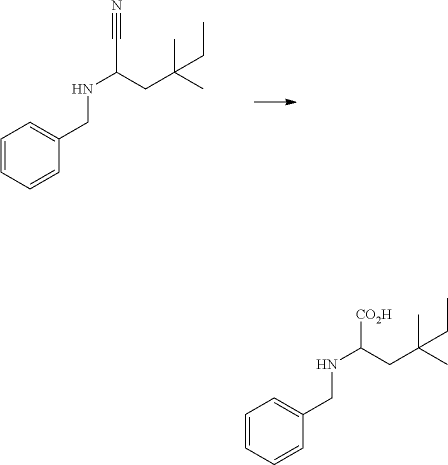 [SMILES: CCC(C)(C)CC(C#N)NCc1ccccc1 ; CCC(C)(C)CC(NCc1ccccc1)C(=O)O]

2-(Benzylamino)-4,4-dimethyl-hexanenitrile (761 mg, 3.1319 mmol) was dissolved in acetic acid (4.2240 g, 4 mL, 70.339 mmol) and hydrochloric acid (24 mL of 12 M, 288.00 mmol) and stirred at 100° C. for 96 hours. The solution was cooled to 10-15° C., the pH was increased to 3-4 with saturated sodium bicarbonate aqueous solution, the mixture was filtered and dried under vacuum to afford 2-(benzylamino)-4,4-dimethyl-hexanoic acid (685 mg, 88%) as a white powder. ¹H NMR (400 MHz, MeOH-d4) δ 7.52-7.42 (m, 5H), 4.15 (d, J 13.2 Hz, 1H), 4.05 (d, J 13.2 Hz, 1H), 3.50 (dd, J 9.3, 3.4 Hz, 1H), 1.94 (dd, J 14.1, 9.4 Hz, 1H), 1.51 (dd, J 13.8, 3.5 Hz, 1H), 1.38-1.29 (m, 2H), 0.93 (s, 6H), 0.84 (t, J 7.5 Hz, 3H); two labile protons missing. ESI-MS m/z calc. 249.17288, found 250.2 (M+1)⁺; Retention time: 1.32 minutes; LC method X.

### Step 7: 2-(Benzylamino)-4,4-dimethyl-hexan-1-ol

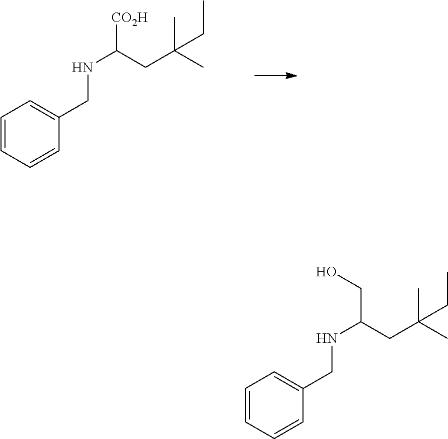 [SMILES: CCC(C)(C)CC(NCc1ccccc1)C(=O)O ; CCC(C)(C)CC(CO)NCc1ccccc1]

To a solution of 2-(benzylamino)-4,4-dimethyl-hexanoic acid (685 mg, 2.7444 mmol) in anhydrous THF (10 mL) was added drop wise at 0° C. borane tetrahydrofuran complex solution in THF (8.3 mL of 1 M, 8.3000 mmol). The reaction was stirred for 30 minutes at 0° C. and then at room temperature for 18 hours. The reaction was cooled down to 0° C. then quenched by the slow addition of methanol (10 mL) and concentrated under reduced pressure. The resulting residue was partitioned between ethyl acetate (50 mL) and 1M sodium hydroxide aqueous solution (40 mL). The biphasic mixture was vigorously stirred until complete solubilization, the layers were separated, and the aqueous layer was extracted ethyl acetate (3×40 mL). The combined organic layers were washed with brine (30 mL), dried over anhydrous sodium sulfate, filtered and concentrated under reduced pressure to afford 2-(benzylamino)-4,4-dimethyl-hexan-1-ol (570 mg, 84%) a colorless oil. ¹H NMR (400 MHz, CDCl₃) δ 7.42-7.28 (m, 5H), 3.82 (d, J 12.7 Hz, 1H), 3.74 (d, J 12.7 Hz, 1H), 3.68 (dd, J 10.5, 3.9 Hz, 1H), 3.25 (dd, J=10.5, 6.4 Hz, 1H), 2.79-2.73 (m, 1H), 1.39-1.34 (m, 2H), 1.30-1.22 (m, 4H), 0.88 (s, 3H), 0.88 (s, 3H), 0.83 (t, J 7.6 Hz, 3H). ESI-MS m/z calc. 235.19362, found 236.2 (M+1)⁺; Retention time: 1.3 minutes; LC method X.

### Step 8: 2-Amino-4,4-dimethyl-hexan-1-ol

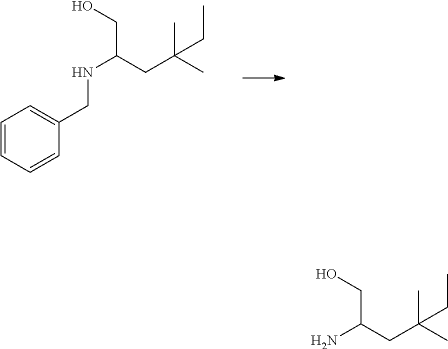

To a degassed solution of 2-(benzylamino)-4,4-dimethyl-hexan-1-ol (560 mg, 2.2603 mmol) in methanol (17 mL) was added 10 wt % palladium on carbon (50% wet) (400 mg, 0.1879 mmol). The mixture was purged with nitrogen for 5 minutes and then hydrogen was bubbled into the solution for 10 minutes. The mixture was then stirred under a hydrogen atmosphere (1 atm.) for 22 hours. The mixture was filtered through a short pad of Celite, the pad was rinsed with methanol (15 mL), and the filtrate was concentrated under reduced pressure to give 2-amino-4,4-dimethyl-hexan-1-ol (305 mg, 88%) as a colorless oil. ¹H NMR (400 MHz, CDCl₃) δ 3.55-3.47 (m, 1H), 3.18 (dd, J 10.3, 8.8 Hz, 1H), 2.98-2.88 (m, 1H), 1.36-1.21 (m, 4H), 1.13 (dd, J 14.5, 6.7 Hz, 1H), 0.91-0.90 (m, 6H), 0.84 (t, J 7.6 Hz, 3H); Two labile protons missing. ESI-MS m/z calc. 145.14667, found 146.2 (M+1)⁺; Retention time: 0.98 minutes; LC method X.

### Step 9: 3-[[4-(2-Amino-4,4-dimethyl-hexoxy)-6-(2,6-dimethylphenyl)pyrimidin-2-yl]sulfamoyl]benzoic acid

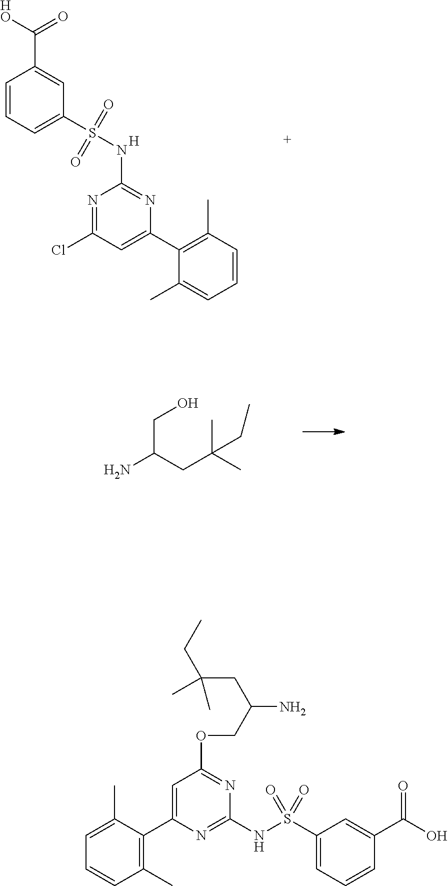

To a solution of 3-[[4-chloro-6-(2,6-dimethylphenyl)pyrimidin-2-yl]sulfamoyl]benzoic acid (870 mg, 2.0820 mmol) and 2-amino-4,4-dimethyl-hexan-1-ol (302 mg, 1.9753 mmol) in 2-methyltetrahydrofuran (8 mL) and N,N-dimethylformamide (0.8 mL) was added sodium tert-butoxide (800 mg, 8.3244 mmol) at 10-15° C. The mixture was stirred for 1 hour. More 3-[[4-chloro-6-(2,6-dimethylphenyl)pyrimidin-2-yl]sulfamoyl]benzoic acid (246 mg, 0.5887 mmol) and sodium tert-butoxide (225 mg, 2.3412 mmol) were added and the mixture was stirred for 45 minutes. The reaction was quenched with 1N hydrochloric acid (20 mL) and extracted with 2-methyltetrahydrofuran (3×20 mL). The combined organic layers were dried over sodium sulfate, filtered and concentrated under reduced pressure. The crude was purified by reverse phase chromatography (Column: 80 g C₁₈. Gradient: 5-100% MeCN in water with 0.1% hydrochloric acid). The fraction containing the product were concentrated to remove acetonitrile, acidified to pH 1-2 with 1N hydrochloric acid and extracted with 2-methyltetrahydrofuran (3×40 mL). The combined organic layers were dried over sodium sulfate, filtered, and concentrated under reduced pressure. The residue was triturated with ethyl acetate (30 mL), filtered and the precipitate was washed with ethyl acetate (2×15 mL). The remaining solid was lyophilized in 1:5 MeCN/water (25 mL) to give 3-[[4-(2-amino-4,4-dimethyl-hexoxy)-6-(2,6-dimethylphenyl)pyrimidin-2-yl]sulfamoyl]benzoic acid (hydrochloride salt) (670 mg, 59%) as a white powder. ¹H NMR (400 MHz, DMSO-d₆) δ 12.99 (br. s., 1H), 8.46 (t, J 1.6 Hz, 1H), 8.36-7.84 (m, 4H), 7.68 (t, J 7.8 Hz, 1H), 7.26 (t, J 7.3 Hz, 1H), 7.13 (d, J 7.6 Hz, 2H), 6.28 (br. s., 1H), 4.32 (dd, J 11.6, 3.1 Hz, 1H), 4.10 (dd, J=11.9, 7.0 Hz, 1H), 3.61-3.53 (m, 1H), 2.01 (br. s., 6H), 1.58 (dd, J 14.7, 7.3 Hz, 1H), 1.47 (dd, J 14.7, 3.7 Hz, 1H), 1.24 (q, J 7.6 Hz, 2H), 0.88 (s, 3H), 0.87 (s, 3H), 0.79 (t, J 7.5 Hz, 3H); two labile protons missing. ESI-MS m/z calc. 526.225, found 527.1 (M+1)⁺; Retention time: 2.54 minutes; LC method Y.

### Step 10: 3-[[4-[2-[[6-[Cyclobutyl(methyl)amino]pyrazin-2-yl]methylamino]-4,4-dimethyl-hexoxy]-6-(2,6-dimethylphenyl)pyrimidin-2-yl]sulfamoyl]benzoic acid

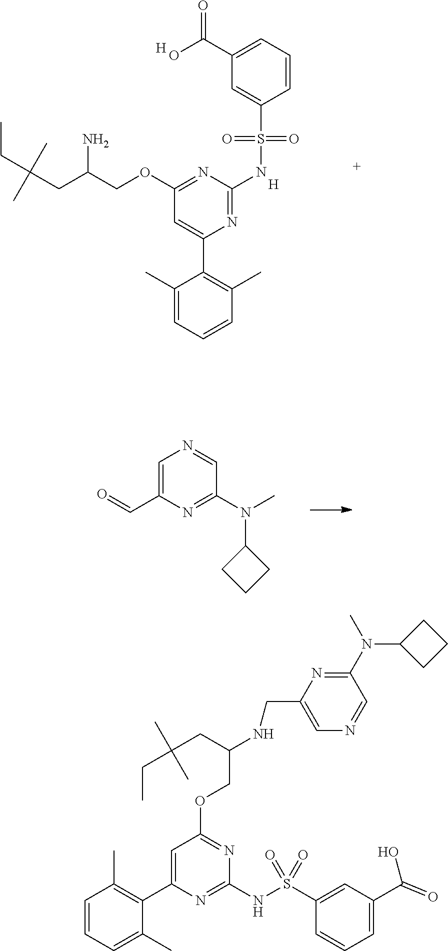

3-[[4-(2-Amino-4,4-dimethyl-hexoxy)-6-(2,6-dimethylphenyl)pyrimidin-2-yl]sulfamoyl]benzoic acid (hydrochloride salt) (80 mg, 0.1421 mmol) was combined with 6-[cyclobutyl(methyl)amino]pyrazine-2-carbaldehyde (30 mg, 0.1569 mmol) in a screwcap vial with DCM (0.4 mL). After stirring for 15 minutes at room temperature sodium triacetoxyborohydride (30 mg, 0.1415 mmol) was added. After an additional 15 minutes an additional portion of sodium triacetoxyborohydride (90 mg, 0.4246 mmol) was added. The reaction was stirred a further 30 minutes then was quenched with 0.2 mL 3M HCl, diluted with methanol and DMSO until the reaction mixture became homogeneous. It was then filtered and purified by preparative HPLC (1-99% ACN in water, HCl modifier, 15 minute run) to give 3-[[4-[2-[[6-[cyclobutyl(methyl)amino]pyrazin-2-yl]methylamino]-4,4-dimethyl-hexoxy]-6-(2,6-dimethylphenyl)pyrimidin-2-yl]sulfamoyl]benzoic acid (hydrochloride salt) (72.3 mg, 69%) upon drying. ESI-MS m/z calc. 701.33594, found 702.9 (M+1)⁺; Retention time: 0.6 minutes; LC method D.

### Step 11: 12-[[6-[Cyclobutyl(methyl)amino]pyrazin-2-yl]methyl]-11-(2,2-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4,6,8(19),14,16-hexaen-13-one

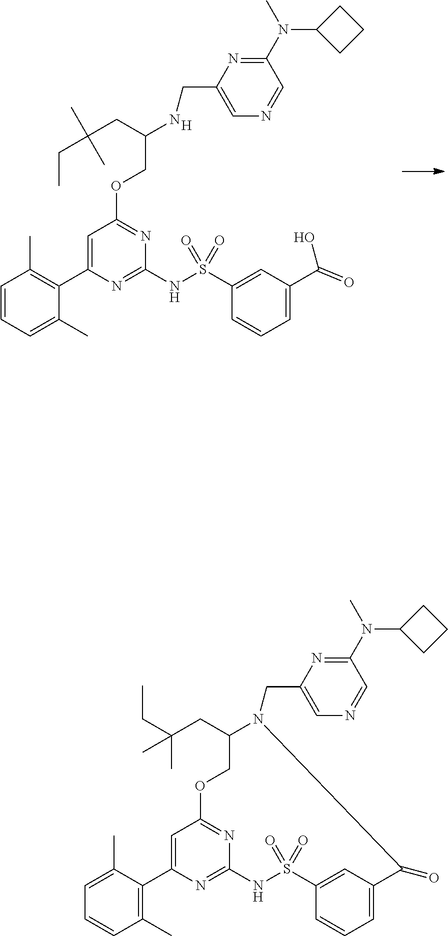

3-[[4-[2-[[6-[Cyclobutyl(methyl)amino]pyrazin-2-yl]methylamino]-4,4-dimethyl-hexoxy]-6-(2,6-dimethylphenyl)pyrimidin-2-yl]sulfamoyl]benzoic acid (hydrochloride salt) (72 mg, 0.09752 mmol) was combined with CDMT (20 mg, 0.1139 mmol) in DMF (5 mL) and cooled to 0° C. in an ice bath. N-methylmorpholine (65 μL, 0.5912 mmol) was added and the reaction was stirred at 0° C. for 10 minutes then the ice bath was removed. The reaction was then stirred at room temperature for 16 hours before it was partitioned between 1M HCl and ethyl acetate. The layers were separated and the aqueous was extracted 2× with ethyl acetate and the combined organics were washed 2× with brine, dried over sodium sulfate and concentrated. The resulting crude material was purified by chromatography on silica gel eluting with a 0-15% methanol in DCM gradient to give 12-[[6-[cyclobutyl(methyl)amino]pyrazin-2-yl]methyl]-11-(2,2-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4,6,8(19),14,16-hexaen-13-one (43.1 mg, 65%). ESI-MS m/z calc. 683.3254, found 684.8 (M+1)⁺; Retention time: 0.77 minutes; LC method D.

### Step 12: 12-[[6-[cyclobutyl(methyl)amino]pyrazin-2-yl]methyl]-11-(2,2-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4,6,8(19),14,16-hexaen-13-one, enantiomer 1 (Compound 1329) and 12-[[6-[cyclobutyl(methyl)amino]pyrazin-2-yl]methyl]-11-(2,2-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4,6,8(19),14,16-hexaen-13-one, enantiomer 2 (Compound 1330)

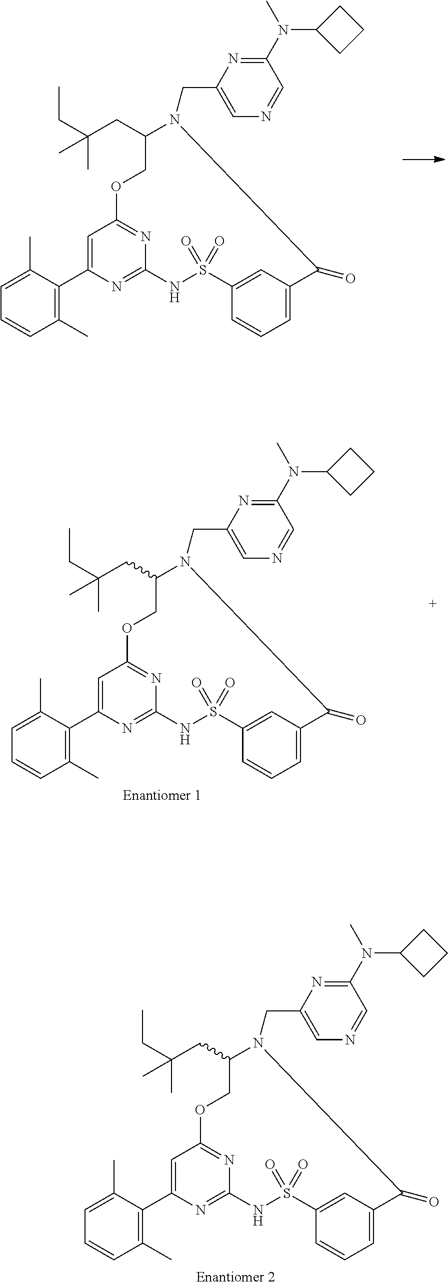 [SMILES: CCC(C)(C)CC1COc2cc(-c3c(C)cccc3C)nc(n2)NS(=O)(=O)c2cccc(c2)C(=O)N1Cc1cncc(N(C)C2CCC2)n1 ; CCC(C)(C)CC1COc2cc(-c3c(C)cccc3C)nc(n2)NS(=O)(=O)c2cccc(c2)C(=O)N1Cc1cncc(N(C)C2CCC2)n1 ; CCC(C)(C)CC1COc2cc(-c3c(C)cccc3C)nc(n2)NS(=O)(=O)c2cccc(c2)C(=O)N1Cc1cncc(N(C)C2CCC2)n1]

The enantiomers of 12-[[6-[cyclobutyl(methyl)amino]pyrazin-2-yl]methyl]-11-(2,2-dimethylbutyl)-6-(2,6-dimethylphenyl)-2,2-dioxo-9-oxa-2?⁶-thia-3,5,12,19-tetrazatricyclo[12.3.1.14,8]nonadeca-1(18),4,6,8(19),14,16-hexaen-13-one (43.1 mg, 0.06302 mmol) were separated by chiral SFC using a ChiralPak AS (21.2×250 mm, 5 μM) column at 40° C. Mobile phase was 30% MeOH (20 mM NH₃), 70% CO₂ at 70 mL/min flow. Concentration of the sample was 20 mg/mL in methanol/DMSO 40:60, injection volume was 200 μL (75° C.) to solubilize through the injection cycle, outlet pressure at 207 bar, and detection wavelength 210 nm. Each diastereomer was collected separately and the resulting products were each further purified by preparative HPLC (1-99% ACN in water, HCl modifier, 15 minute run) to give Enantiomer 1 (Peak 1 SFC) 12-[[6-[cyclobutyl(methyl)amino]pyrazin-2-yl]methyl]-11-(2,2-dimethylbutyl)-6-(2,6-dimethylphenyl)-2,2-dioxo-9-oxa-2?⁶-thia-3,5,12,19-tetrazatricyclo[12.3.1.14,8]nonadeca-1(18),4,6,8(19),14,16-hexaen-13-one (3.3 mg, 7%) ¹H NMR (400 MHz, Chloroform-d) δ 8.86 (d, J 1.8 Hz, 1H), 8.05 (d, J 7.9 Hz, 1H), 7.88 (t, J 9.4 Hz, 3H), 7.65 (t, J 7.8 Hz, 1H), 7.22 (t, J 7.6 Hz, 1H), 7.08 (d, J 7.6 Hz, 2H), 6.27 (s, 1H), 5.46 (dd, J 11.1, 4.3 Hz, 1H), 5.09 (d, J 15.5 Hz, 1H), 4.52 (p, J 8.4 Hz, 1H), 4.23 (t, J 9.2 Hz, 1H), 4.14 (d, J 15.6 Hz, 1H), 4.06 (t, J 11.4 Hz, 1H), 3.17 (s, 3H), 2.35-2.26 (m, 2H), 2.26-2.17 (m, 2H), 2.03 (s, 6H), 1.78-1.74 (m, 2H), 1.59-1.56 (m, 1H), 0.95 (dd, J 14.1, 7.3 Hz, 1H), 0.89-0.84 (m, 1H), 0.83-0.78 (m, 1H), 0.60 (s, 3H), 0.58-0.52 (m, 6H). Sulfonamide N-Hnot visible. ESI-MS m/z calc. 683.3254, found 684.8 (M+1)⁺; Retention time: 1.92 minutes and Enantiomer 2 (Peak 2 SFC) 12-[[6-[cyclobutyl(methyl)amino]pyrazin-2-yl]methyl]-11-(2,2-dimethylbutyl)-6-(2,6-dimethylphenyl)-2,2-dioxo-9-oxa-2λ⁶-thia-3,5,12,19-tetrazatricyclo[12.3.1.14,8]nonadeca-1(18),4,6,8(19),14,16-hexaen-13-one (3.1 mg, 7%)¹H NMR (400 MHz, Chloroform-d) δ 8.86 (t, J 1.7 Hz, 1H), 8.10 (d, J 7.9 Hz, 1H), 7.92-7.83 (m, 3H), 7.67 (t, J 7.8 Hz, 1H), 7.22 (t, J 7.6 Hz, 1H), 7.08 (d, J 7.6 Hz, 2H), 6.29 (s, 1H), 5.47 (dd, J 11.1, 4.2 Hz, 1H), 5.08 (d, J 15.5 Hz, 1H), 4.54-4.46 (m, 1H), 4.25 (s, 1H), 4.16 (d, J 15.7 Hz, 1H), 4.06 (t, J=11.4 Hz, 1H), 3.19 (s, 3H), 2.36-2.28 (m, 2H), 2.25-2.18 (m, 2H), 2.03 (s, 6H), 1.77-1.73 (m, 2H), 1.61-1.59 (m, 1H), 0.98-0.92 (m, 1H), 0.88-0.84 (m, 2H), 0.60 (s, 3H), 0.59-0.53 (m, 6H). (Sulfonamide —NH not visible) ESI-MS m/z calc. 683.3254, found 684.8 (M+1)⁺; Retention time: 1.92 minutes LC method A.

### Example 168: Preparation of Compound 1331

### Step 1: 3-[[4-[(2R)-2-Amino-3-cyclohexyl-propoxy]-6-(2,6-dimethylphenyl)pyrimidin-2-yl]sulfamoyl]benzoic acid

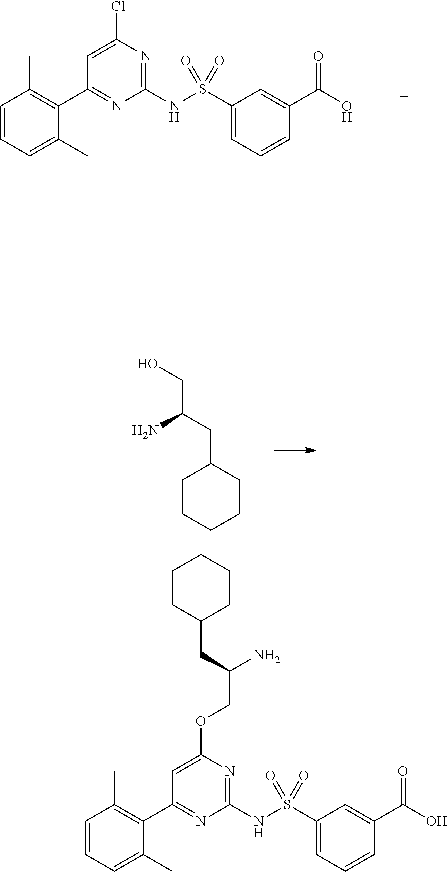

3-[[4-chloro-6-(2,6-Dimethylphenyl)pyrimidin-2-yl]sulfamoyl]benzoic acid (500 mg, 1.197 mmol) was combined with (2R)-2-amino-3-cyclohexyl-propan-1-ol (198 mg, 1.259 mmol) in THF (2.5 mL) and stirred vigorously for 5 minutes at room temperature. Sodium tert-butoxide (600 mg, 6.243 mmol) was added in one portion and the reaction mixture became very warm to the touch. Stirring was continued without any external heating for 15 minutes. The reaction mixture was then partitioned between 1M HCl and ethyl acetate. The layers were separated and the aqueous was extracted an additional 2× with ethyl acetate. The aqueous was diluted with an equal volume of brine and extracted an additional time with ethyl acetate. The combined organics were washed with brine, dried over sodium sulfate, and concentrated to give as an offwhite solid, 3-[[4-[(2R)-2-amino-3-cyclohexyl-propoxy]-6-(2,6-dimethylphenyl)pyrimidin-2-yl]sulfamoyl]benzoic acid (hydrochloride salt) (699.7 mg, 83%). ESI-MS m/z calc. 538.225, found 539.5 (M+1)⁺; Retention time: 0.51 minutes; LC method A.

### Step 2: 3-[[4-[(2R)-2-[(6-Chloropyrazin-2-yl)methylamino]-3-cyclohexyl-propoxy]-6-(2,6-dimethylphenyl)pyrimidin-2-yl]sulfamoyl]benzoic acid

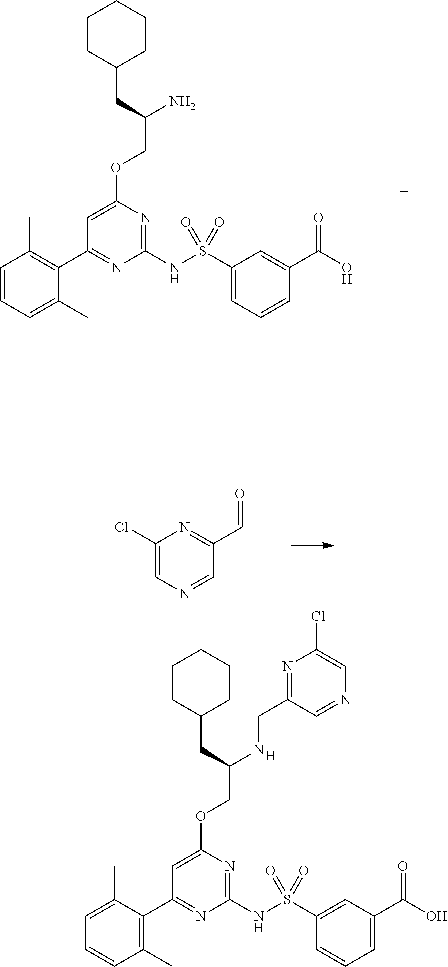

3-[[4-[(2R)-2-Amino-3-cyclohexyl-propoxy]-6-(2,6-dimethylphenyl)pyrimidin-2-yl]sulfamoyl]benzoic acid (hydrochloride salt) (250 mg, 0.3564 mmol) was combined with 6-chloropyrazine-2-carbaldehyde (56 mg, 0.3929 mmol) in DCM (2 mL) and stirred for 15 minutes. Sodium triacetoxyborohydride (75 mg, 0.3539 mmol) was added and the reaction was stirred for 10 minutes at room temperature so a second portion of sodium triacetoxyborohydride (226 mg, 1.066 mmol) was added. After a further 30 minutes the reaction was quenched with 3 M HCl then diluted with methanol and DMSO until the reaction mixture became homogeneous. The reaction mixture was filtered and purified by preparative HPLC (1-99% ACN in water, 15 minute run) to give 3-[[4-[(2R)-2-[(6-chloropyrazin-2-yl)methylamino]-3-cyclohexyl-propoxy]-6-(2,6-dimethylphenyl)pyrimidin-2-yl]sulfamoyl]benzoic acid (hydrochloride salt) (143 mg, 57%). ESI-MS m/z calc. 664.22345, found 665.7 (M+1)⁺; Retention time: 0.57 minutes; LC method D.

### Step 3: (11R)-12-[(6-Chloropyrazin-2-yl)methyl]-11-(cyclohexylmethyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one

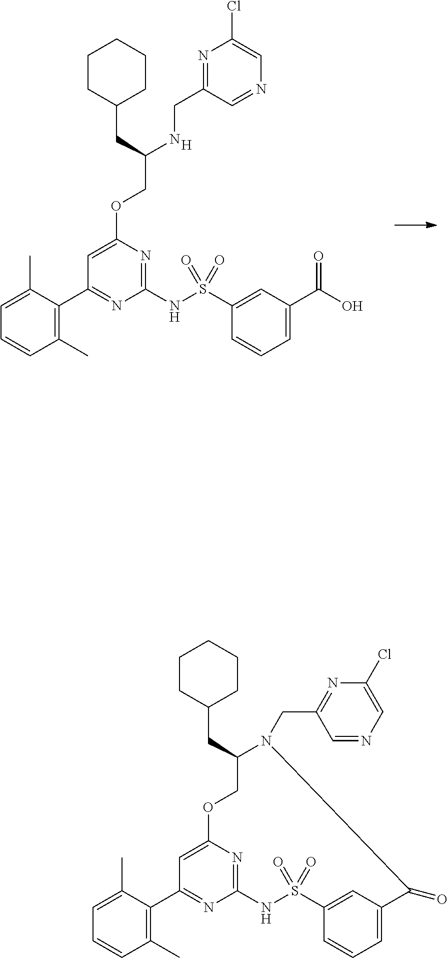

3-[[4-[(2R)-2-[(6-Chloropyrazin-2-yl)methylamino]-3-cyclohexyl-propoxy]-6-(2,6-dimethylphenyl)pyrimidin-2-yl]sulfamoyl]benzoic acid (hydrochloride salt) (143 mg, 0.2038 mmol) was combined with CDMT (43 mg, 0.2449 mmol) in DMF (10 mL) and cooled to 0° C. in an ice bath. N-methylmorpholine (140 μL, 1.273 mmol) was added and the reaction was stirred at 0° C. for 10 minutes then the ice bath was removed. The reaction was then stirred at room temperature for 16 hours before it was partitioned between 1M HCl and ethyl acetate. The layers were separated and the aqueous was extracted 2× with ethyl acetate and the combined organics were washed 2× with brine, dried over sodium sulfate and concentrated. The resulting crude material was purified by chromatography on silica gel eluting with a 0-15% methanol in DCM gradient to give (11R)-12-[(6-chloropyrazin-2-yl)methyl]-11-(cyclohexylmethyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (95.9 mg, 73%). ESI-MS m/z calc. 646.2129, found 647.7 (M+1)⁺; Retention time: 0.78 minutes; LC method D.

### Step 4: (11R)-11-(Cyclohexylmethyl)-6-(2,6-dimethylphenyl)-12-[[6-[(2R)-2-methylpyrrolidin-1-yl]pyrazin-2-yl]methyl]-2,2-dioxo-9-oxa-2⁶-thia-3,5,12,19-tetrazatricyclo[12.3.1.14,8]nonadeca-1(18),4(19),5,7,14,16-hexaen-13-one (Compound 1331)

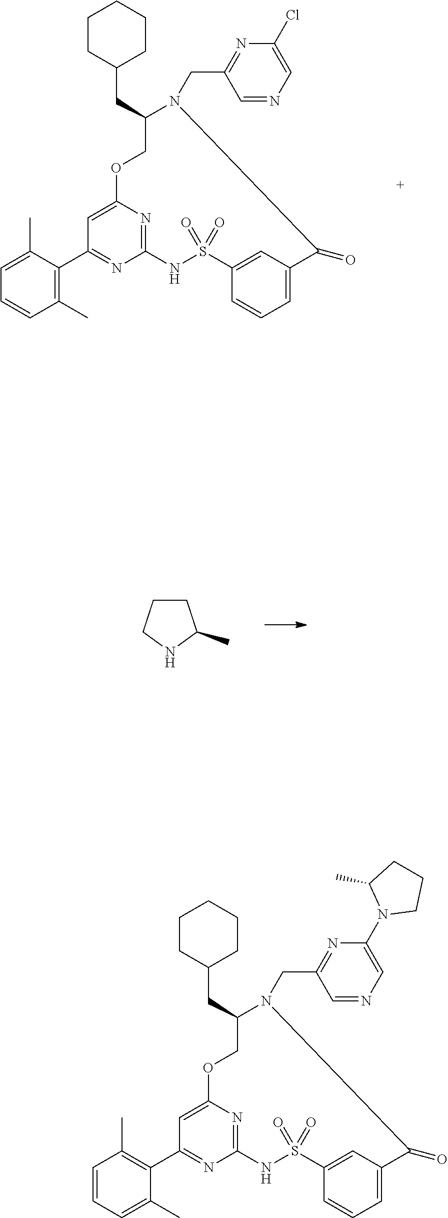

(11R)-12-[(6-Chloropyrazin-2-yl)methyl]-11-(cyclohexylmethyl)-6-(2,6-dimethylphenyl)-2,2-dioxo-9-oxa-2?⁶-thia-3,5,12,19-tetrazatricyclo[12.3.1.14,8]nonadeca-1(18),4(19),5,7,14,16-hexaen-13-one (15 mg, 0.02318 mmol) was combined with (2R)-2-methylpyrrolidine (8 mg, 0.09395 mmol) and potassium carbonate (35 mg, 0.2532 mmol) (325 mesh), DMSO (0.3 mL), and dioxane (0.1 mL) in a screwcap and heated at 120° C. for 6 hours. The reaction mixture was then cooled to room temperature, diluted with methanol, filtered, and purified by preparative HPLC (1-99% ACN in water, HCl modifier 15 minute run) to give (11R)-11-(cyclohexylmethyl)-6-(2,6-dimethylphenyl)-12-[[6-[(2R)-2-methylpyrrolidin-1-yl]pyrazin-2-yl]methyl]-2,2-dioxo-9-oxa-2?⁶-thia-3,5,12,19-tetrazatricyclo[12.3.1.14,8]nonadeca-1(18),4(19),5,7,14,16-hexaen-13-one (7.4 mg, 45%). ESI-MS m/z calc. 695.3254, found 696.8 (M+1)⁺; Retention time: 1.92 minutes; LC method A. ¹H NMR (400 MHz, Chloroform-d) δ 8.81 (d, J 1.8 Hz, 1H), 8.00 (d, J 7.9 Hz, 1H), 7.86 (s, 1H), 7.82 (dt, J 7.8, 1.3 Hz, 1H), 7.76 (s, 1H), 7.62 (t, J 7.8 Hz, 1H), 7.22 (t, J 7.6 Hz, 1H), 7.07 (d, J=7.6 Hz, 2H), 6.23 (s, 1H), 5.47 (dd, J 11.1, 4.1 Hz, 1H), 5.09 (d, J=15.5 Hz, 1H), 4.29-4.17 (m, 2H), 4.13-4.01 (m, 2H), 3.66 (td, J=8.3, 7.5, 4.3 Hz, 1H), 3.53-3.42 (m, 1H), 2.22-2.06 (m, 3H), 2.06 (s, 6H), 1.79-1.75 (m, 1H), 1.67-1.65 (m, 1H), 1.64-1.62 (m, 1H), 1.61-1.59 (m, 1H), 1.54-1.49 (m, 1H), 1.46-1.36 (m, 2H), 1.27 (d, J 6.3 Hz, 3H), 1.20 (d, J 12.3 Hz, 1H), 1.14-1.07 (m, 1H), 1.04-0.97 (m, 2H), 0.89 (qd, J 12.1, 3.4 Hz, 2H), 0.40-0.27 (m, 1H). (Sulfonamide —NH not visible)

### Example 169: Preparation of Compound 1332

### Step 1: Benzyl N-(2-oxocyclobutyl)carbamate

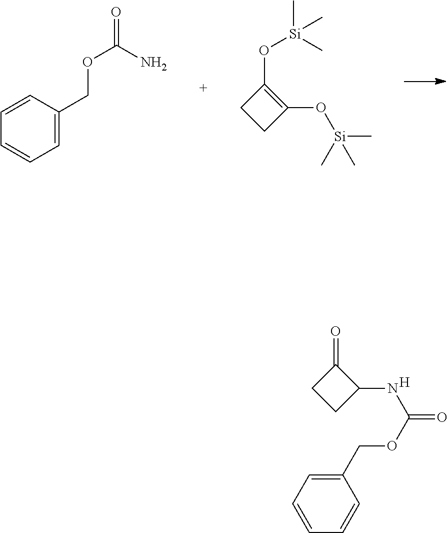

In a sealed tube, 1,2-bis(trimethylsiloxy)cyclobutene (2 g, 8.6786 mmol) was added to benzyl carbamate (1.6 g, 5.3872 mL, 10.585 mmol) in HCl (in diethylether) (30 mL of 2 M, 60.000 mmol) at 0° C. The sealed tube was capped and heated at 80° C. for 4 h. The tube was cooled to 0° C. before opening it. The content was concentrated under reduced pressure and the resulting residue was purified on silica gel using a 0 to 50% ethyl acetate in heptanes to provide benzyl N-(2-oxocyclobutyl)carbamate (1.4 g, 70%) as a clear yellow oil ¹H NMR (400 MHz, CDCl₃) δ 7.39-7.33 (m, 5H), 5.40-5.20 (m, 1H), 5.11 (s, 2H), 4.89 (q, J 8.5 Hz, 1H), 3.01-2.79 (m, 2H), 2.54-2.38 (m, 1H), 2.10-1.97 (m, 1H). ESI-MS m/z calc. 219.08954, found 220.2 (M+1)⁺; Retention time: 1.55 minutes; LC method X.

### Step 2: Benzyl N-(2-hydroxycyclobutyl)carbamate

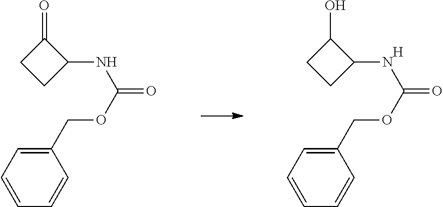

Sodium borohydride (444 mg, 0.4698 mL, 11.736 mmol) was added in small portions to a solution of benzyl N-(2-oxocyclobutyl)carbamate (1.4 g, 6.0665 mmol) in methanol (20 mL) at 0° C. and left stirring for 30 min. The reaction was then left stirring at room temperature for another 30 min then it was concentrated under reduced pressure and the resulting then partitioned between DCM (100 mL) and water (25 mL). The aqueous phase was separated and extracted with DCM (50 mL). The combined organic phases were dried with sodium sulfate, filtered, and concentrated under reduced pressure. The resulting residue was purified on silica gel using 0 to 50% ethyl acetate in heptanes to provide benzyl N-(2-hydroxycyclobutyl)carbamate (1.03 g, 77%) as a clear oil ¹H NMR (400 MHz, CDCl₃) δ 7.46-7.30 (m, 5H), 5.10 (s, 2H), 5.01 (br. s., 1H), 4.08-3.95 (m, 1H), 3.84-3.70 (m, 1H), 2.95 (br. s., 1H), 2.21-2.01 (m, 2H), 1.72-1.52 (m, 1H, overlap with water signal), 1.39-1.28 (m, 1H). ESI-MS m/z calc. 221.1052, found 222.2 (M+1)⁺; Retention time: 1.47 minutes; LC method X.

### Step 3: tert-Butyl 3-[2-(benzyloxycarbonylamino)cyclobutoxy]propanoate

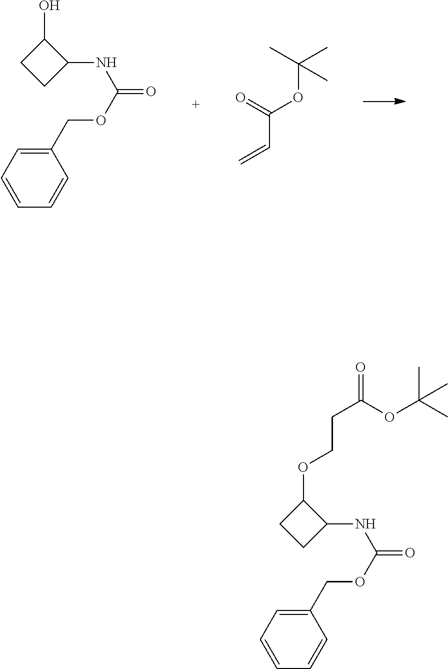

Cesium carbonate (1.5 g, 4.6038 mmol) was added to a solution of benzyl N-(2-hydroxycyclobutyl)carbamate (1 g, 4.5197 mmol) and tert-butyl prop-2-enoate (11.7 g, 91.286 mmol) in t-butanol (20 mL) in a sealed tube. The tube was closed, and the reaction was heated to 40° C. for 2 days. The reaction mixture was cooled down to room temperature and DCM (20 mL) was added and the mixture was filtered. The solid was rinsed with DCM (10 mL) and the combined filtrates were concentrated under reduced pressure. The resulting residue was purified was purified by reverse phase chromatography on 50 g C₁₈ column using a 5 to 100% gradient of ACN in acidic water (0.1% formic acid content) to provide tert-butyl 3-[2-(benzyloxycarbonylamino)cyclobutoxy]propanoate (1.1 g, 66%) as a thick clear oil. ESI-MS m/z calc. 349.1889, found 372.2 (M+23)⁺; Retention time: 1.85 minutes; LC method X.

### Step 4: 3-[2-(Benzyloxycarbonylamino)cyclobutoxy]propanoic acid

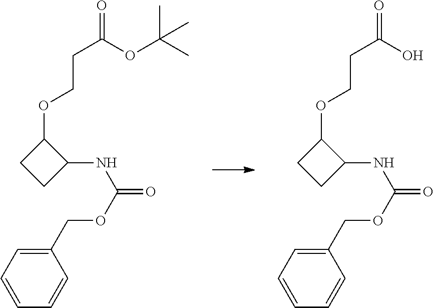 [SMILES: CC(C)(C)OC(=O)CCOC1CCC1NC(=O)OCc1ccccc1 ; O=C(O)CCOC1CCC1NC(=O)OCc1ccccc1]

TFA (22.200 g, 15 mL, 194.70 mmol) was added to a solution of tert-butyl 3-[2-(benzyloxycarbonylamino)cyclobutoxy]propanoate (1.1 g, 3.1481 mmol) and triethylsilane (1.0920 g, 1.5 mL, 9.3914 mmol) in DCM (15 mL) at room temperature. The reaction mixture was stirred at this temperature for 1 h. Then was concentrated under reduced pressure. The resulting residue was suspended in water: acetonitrile 1:1 (5 mL) then distilled water (30 mL) was added and the resulting suspension was concentrated under reduced pressure and the resulting oil was submitted to the same process over again (2×) to provide 3-[2-(benzyloxycarbonylamino)cyclobutoxy]propanoic acid (923 mg, 100%) as a clear oil which solidified on standing ¹H NMR (400 MHz, CDCl₃) δ 7.45-7.29 (m, 5H), 6.68 (br. s., 1H), 5.25-5.02 (m, 3H), 4.07-3.90 (m, 1H), 3.86-3.63 (m, 3H), 2.66-2.51 (m, 2H), 2.19-1.98 (m, 2H), 1.67-1.48 (m, 1H), 1.47-1.31 (m, 1H). ESI-MS m/z calc. 293.1263, found 294.2 (M+1)⁺; Retention time: 1.53 minutes; LC method X.

### Step 5: 3-(2-Aminocyclobutoxy)propanoic acid

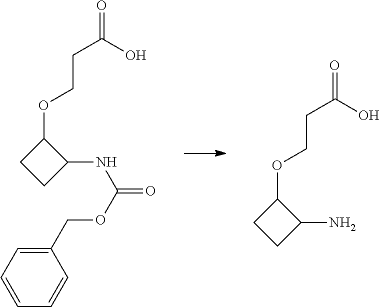 [SMILES: O=C(O)CCOC1CCC1NC(=O)OCc1ccccc1 ; NC1CCC1OCCC(=O)O]

Palladium on carbon (740 mg, 5% w/w, 0.3477 mmol) was added to a solution of 3-[2-(benzyloxycarbonylamino)cyclobutoxy]propanoic acid (1.02 g, 3.4775 mmol) in methanol (10 mL). Hydrogen gas was injected continuously into the stirring suspension for 1 h using a fine needle. A small amount of Celite was added reaction mixture and this suspension was filtered on Celite. The solid was washed with methanol. The filtrates were combined and concentrated under reduced pressure to provide pure 3-(2-aminocyclobutoxy)propanoic acid (550 mg, 99%) as a pale yellow solid ¹H NMR (400 MHz, DMSO-d₆) δ 6.46 (br. s., 3H), 3.69 (q, J 7.3 Hz, 1H), 3.60 (t, J 6.5 Hz, 2H), 3.21-3.12 (m, 1H), 2.38-2.30 (m, 2H), 1.98 (q, J 9.1 Hz, 1H), 1.93-1.84 (m, 1H), 1.46-1.34 (m, 1H), 1.33-1.16 (m, 1H). ESI-MS m/z calc. 159.08954, found 160.2 (M+1)⁺; Retention time: 0.23 minutes; LC method X.

### Step 6: 2-Oxa-6-azabicyclo[5.2.0]nonan-5-one

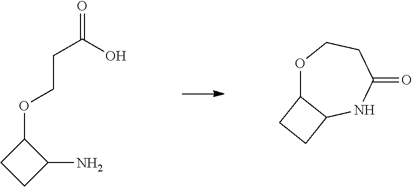 [SMILES: NC1CCC1OCCC(=O)O ; O=C1CCOC2CCC2N1]

T3P (in ethyl acetate) (5.3450 g, 10 mL of 50% w/w, 8.3993 mmol) was added to a solution of 3-(2-aminocyclobutoxy)propanoic acid (571 mg, 3.5871 mmol) and triethyl amine (2.9040 g, 4 mL, 28.698 mmol) in dioxane (700 mL). This reaction mixture was left to stir overnight. The reaction mixture was concentrated under reduced pressure and the resulting residue was purified by reverse phase chromatography on a C₁₈ column using 5-20% acetonitrile in acidic water (0.1% formic acid in water) to provide 3 batches of different purities of 2-oxa-6-azabicyclo[5.2.0]nonan-5-one (25 mg, 5%) as white powder ¹H NMR (400 MHz, CDCl₃) δ 6.26 (br. s., 1H), 3.99 (ddd, J 12.4, 5.2, 2.9 Hz, 1H), 3.73 (t, J 11.9 Hz, 1H), 3.67-3.58 (m, 1H), 3.46-3.38 (m, 1H), 3.00 (ddd, J 15.0, 11.8, 2.8 Hz, 1H), 2.53 (ddd, J 15.1, 5.2, 1.5 Hz, 1H), 2.15 (dt, J 10.3, 7.8 Hz, 1H), 2.11-2.03 (m, 1H), 1.80 (qd, J 10.1, 8.3 Hz, 1H), 1.48 (qd, J 10.1, 7.7 Hz, 1H). ESI-MS m/z calc. 141.079, found 142.2 (M+1)⁺; Retention time: 0.61 minutes, fraction 2: 2-oxa-6-azabicyclo[5.2.0]nonan-5-one (56 mg, 11%) as a white powder ESI-MS m/z calc. 141.079, found 142.2 (M+1)⁺; Retention time: 0.61 minutes and fraction 3: 2-oxa-6-azabicyclo[5.2.0]nonan-5-one (55 mg, 10%) as a white powder ESI-MS m/z calc. 141.079, found 142.2 (M+1)⁺; Retention time: 0.61 minutes; LC method X.

### Step 7: 2-Oxa-6-azabicyclo[5.2.0]nonane

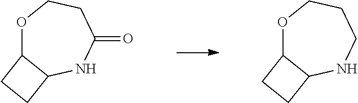 [SMILES: O=C1CCOC2CCC2N1 ; C1CNC2CCC2OC1]

LAH (26 mg, 0.6850 mmol) was added to 2-oxa-6-azabicyclo[5.2.0]nonan-5-one (55 mg, 0.3896 mmol) in THF (5 mL) and heated at 60° C. overnight. The reaction mixture was cooled to room temperature, quenched with water (0.1 mL) aqueous 15% NaOH (0.1 mL) and magnesium sulfate (100 mg) and the reaction mixture was left stirring 1 h at room temperature. The reaction mixture was filtered on Celite and the cake was washed with MeTHF (50 mL). The filtrate was extracted with 0.2 N HCl (2×10 mL). The aqueous phases were combined and concentrated under reduced pressure and then left under high vacuum for 2 h to provide crude 2-oxa-6-azabicyclo[5.2.0]nonane (hydrochloride salt) (40 mg, 50%) as a dark oil ¹H NMR (400 MHz, DMSO-d₆) δ 9.60 (br. s., 1H), 9.39 (br. s., 1H), 4.13-4.00 (m, 1H), 3.96-3.82 (m, 1H), 3.64-3.43 (m, 3H, overlap with the water signal), 3.39-3.15 (m, 2H), 2.10-1.89 (m, 3H), 1.83-1.69 (m, 1H), 1.69-1.51 (m, 1H). ESI-MS m/z calc. 127.09972, found 128.2 (M+1)⁺; Retention time: 0.21 minutes; LC method X.

### Step 8: (11R)-6-(2,6-Dimethylphenyl)-11-(2,2-dimethylpropyl)-12-[[6-(2-oxa-6-azabicyclo[5.2.0]nonan-6-yl)pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1332)

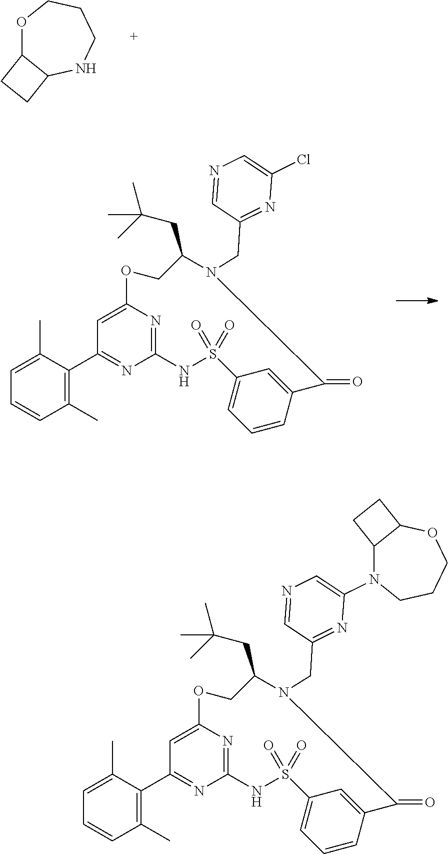 [SMILES: C1CNC2CCC2OC1 ; Cc1cccc(C)c1-c1cc2nc(n1)NS(=O)(=O)c1cccc(c1)C(=O)N(Cc1cncc(Cl)n1)[C@H](CC(C)(C)C)CO2 ; Cc1cccc(C)c1-c1cc2nc(n1)NS(=O)(=O)c1cccc(c1)C(=O)N(Cc1cncc(N3CCCOC4CCC43)n1)[C@H](CC(C)(C)C)CO2]

In a microwave vial, finely grounded potassium carbonate (50 mg, 0.3618 mmol) was added to a solution of (11R)-12-[(6-chloropyrazin-2-yl)methyl]-6-(2,6-dimethylphenyl)-11-(2,2-dimethylpropyl)-2,2-dioxo-9-oxa-2λ⁶-thia-3,5,12,19-tetrazatricyclo[12.3.1.14,8]nonadeca-1(18),4(19),5,7,14,16-hexaen-13-one (35 mg, 0.0563 mmol) and 2-oxa-6-azabicyclo[5.2.0]nonane (hydrochloride salt) (32 mg, 0.1564 mmol) in DMSO (100 μL). The vial was capped and left stirring at room temperature for 30 min then heated in an oil bath from 25 to 120° C. in −45 min and left stirring at 120° C. temperature overnight. The reaction mixture was diluted with a small amount of DMSO, filtered and injected onto a C₁₈ (15.5 g) reverse phase column for purification using a 5 to 100% gradient of acetonitrile in acidic water (0.1% formic acid content) to provide (11R)-6-(2,6-dimethylphenyl)-11-(2,2-dimethylpropyl)-12-[[6-(2-oxa-6-azabicyclo[5.2.0]nonan-6-yl)pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one (1:1 mixture of diastereomers, 25.6 mg, 63%) as a beige fluffy powder after lyophilization. ¹H NMR (400 MHz, DMSO-d₆) δ 13.02 (br. s., 1H), 8.68 (s, 1H), 8.01-7.92 (m, 1H), 7.87 (s, 1H), 7.80-7.63 (m, 3H), 7.31-7.22 (m, 1H), 7.19-7.07 (m, 2H), 6.44 (br. s., 1H), 5.45-5.30 (m, 1H), 4.91-4.68 (m, 2H), 4.46 (dd, J 15.8, 4.3 Hz, 1H), 4.35-4.21 (m, 1H), 4.10-3.99 (m, 1H), 3.96-3.87 (m, 2H), 3.86-3.74 (m, 1H), 3.67-3.44 (m, 2H), 2.38 (q, J 8.6 Hz, 1H), 2.26-1.84 (m, 8H), 1.83-1.68 (m, 2H), 1.61 (quin, J 9.5 Hz, 1H), 1.41 (dd, J 14.9, 5.4 Hz, 1H), 1.30-1.16 (m, 1H), 0.56 (s, 9H). ESI-MS m/z calc. 711.3203, found 712.2 (M+1)⁺; Retention time: 4.36 minutes; LC method Y.

### Example 170: Preparation of Compound 1333

### Step 1: 2-[(4-Methoxyphenyl)methylamino]acetonitrile

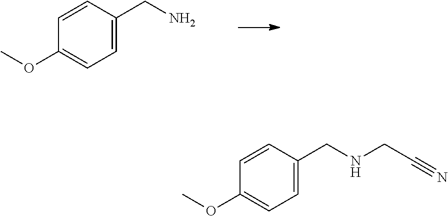

To a solution of (4-methoxyphenyl)methanamine (1.0500 g, 1 mL, 7.6542 mmol) in THF (15 mL) at 0° C., triethylamine (871.20 mg, 1.2 mL, 8.6095 mmol) was added. bromoacetonitrile (1.0320 g, 0.6 mL, 8.6037 mmol) was slowly added and stirred for 10 minutes. The reaction mixture was slowly warmed to room temperature and stirred for 16 h. After 16 h, the reaction mixture was concentrated in vacuo, diluted with ethyl acetate (10 mL) and water (10 mL). The aqueous layer was extracted with ethyl acetate (2×20 mL). The combined organic layers were washed with brine (20 mL), dried with sodium sulfate, filtered and concentrated in vacuo. The residue was purified by silica gel chromatography in 60% ethyl acetate in hexanes to furnish a clear oil. To a suspension of clear oil in diethyl ether, HCl/Dioxane (2.7 mL of 4 M, 10.800 mmol) was added and the resulting mixture was filtered and the filter cake was washed with diethyl ether to afford 2-[(4-methoxyphenyl)methylamino]acetonitrile (hydrochloride salt) (1.45 g, 88%) as a pale yellow solid. ¹H NMR (400 MHz, DMSO) δ 10.51 (s, 1H), 7.48 (d, J 6.5 Hz, 2H), 6.97 (d, J 6.5 Hz, 2H), 4.24 (s, 2H), 4.12 (s, 2H), 3.76 (s, 3H); ESI-MS m/z calc. 176.09496, found 177.0 (M+1)⁺; Retention time: 1.23 minutes; LC method T.

### Step 2: 3,5-Dichloro-1-[(4-methoxyphenyl)methyl]pyrazin-2-one

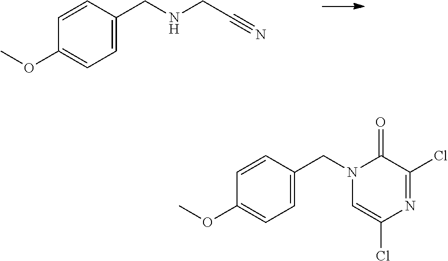 [SMILES: COc1ccc(CNCC#N)cc1 ; COc1ccc(Cn2cc(Cl)nc(Cl)c2=O)cc1]

To a solution of 2-[(4-methoxyphenyl)methylamino]acetonitrile (hydrochloride salt) (1.45 g, 6.8179 mmol) in chlorobenzene (12 mL) at room temperature, oxalyl chloride (2.6190 g, 1.8 mL, 20.634 mmol) was added and the reaction was stirred for 30 minutes. Triethylamine (hydrochloride salt) (4.6 g, 6.3361 mL, 33.418 mmol) was added and the reaction was stirred for 16 h. The reaction mixture is diluted with DCM (100 mL) and washed with water (2×50 mL). The organic layer was washed with brine (50 mL), dried with sodium sulfate, filtered and concentrated in vacuo. The residue was purified by silica gel chromatography in 70% ethyl acetate in hexanes to furnish to give 3,5-dichloro-1-[(4-methoxyphenyl)methyl]pyrazin-2-one (1.28 g, 63%) as a pale yellow oil. ¹H NMR (500 MHz, DMSO) δ 8.12 (s, 1H), 7.21 (d, 2H), 6.83 (d, 2H), 4.92 (s, 2H), 3.63 (s, 3H). ESI-MS m/z calc. 284.01193, found 285.2 (M+1)⁺; Retention time: 2.69 minutes; LC method T.

### Step 3: 5-Chloro-1-[(4-methoxyphenyl)methyl]-3-(3-methylbut-1-ynyl)pyrazin-2-one

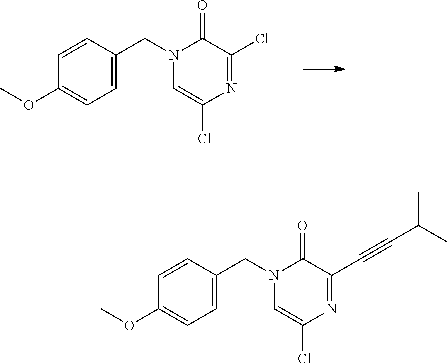 [SMILES: COc1ccc(Cn2cc(Cl)nc(Cl)c2=O)cc1 ; COc1ccc(Cn2cc(Cl)nc(C#CC(C)C)c2=O)cc1]

To a microwave vial 3,5-dichloro-1-[(4-methoxyphenyl)methyl]pyrazin-2-one (1.08 g, 3.7878 mmol), bis(triphenylphosphine)palladium(II) dichloride (28 mg, 0.0399 mmol), CuI (22 mg, 0.1155 mmol) in DMF (6 mL) and Et₃N (6 mL), isopropylacetylene (396.00 mg, 0.6 mL, 5.8135 mmol) was added and sealed. The microwave vial was irradiated at 80° C. for 10 min. The reaction mixture was allowed to cool, was diluted with DCM (50 mL) and washed with water (2×50 mL). The organic layer was washed with brine, dried with sodium sulfate, filtered and concentrated in vacuo. The residue was purified by silica gel chromatography in 50% ethyl acetate in hexanes to furnish to give 5-chloro-1-[(4-methoxyphenyl)methyl]-3-(3-methylbut-1-ynyl)pyrazin-2-one (1.02 g, 83%) as a pale yellow solid. ¹H NMR (500 MHz, CDCl₃) δ 7.28 (d, J 8.6 Hz, 2H), 7.10 (s, 1H), 6.90 (d, J 6.6 Hz, 2H), 4.99 (s, 2H), 3.81 (s, 3H), 2.89 (dt, J=13.7, 6.9 Hz, 1H), 1.30 (d, 6H). ESI-MS m/z calc. 316.09787, found 317.1 (M+1)⁺; Retention time: 3.12 minutes; LC method T.

### Step 4: 2-Chloro-6-isopropyl-furo[2,3-b]pyrazine

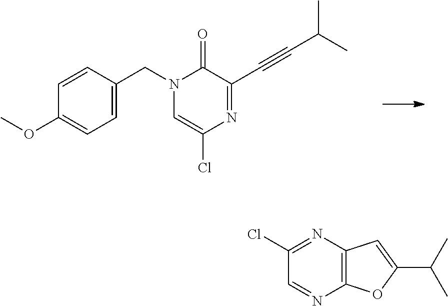 [SMILES: COc1ccc(Cn2cc(Cl)nc(C#CC(C)C)c2=O)cc1 ; CC(C)c1cc2nc(Cl)cnc2o1]

To a solution of 5-chloro-1-[(4-methoxyphenyl)methyl]-3-(3-methylbut-1-ynyl)pyrazin-2-one (970 mg, 3.0620 mmol) in DCM (20 mL), trifluoromethanesulfonate (20 mg, 0.0778 mmol) and TFA (2.9600 g, 2 mL, 25.960 mmol) were added and the reaction was stirred at room temperature for 30 minutes. The residue was concentrated in vacuo and purified by silica gel chromatography in 30% ethyl acetate in hexanes to furnish 2-chloro-6-isopropyl-furo[2,3-b]pyrazine (510 mg, 84%) as a pale yellow solid. ¹H NMR (500 MHz, CDCl₃) δ 8.15 (s, 1H), 6.56 (s, 1H), 3.17 (dt, J 13.8, 6.9 Hz, 1H), 1.40 (d, J 6.9 Hz, 6H). ESI-MS m/z calc. 196.04034, found 197.1 (M+1)⁺; Retention time: 3.14 minutes; LC method T.

### Step 5: Methyl 6-isopropylfuro[2,3-b]pyrazine-2-carboxylate

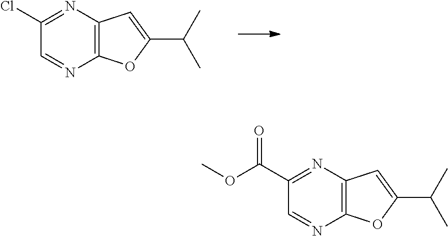 [SMILES: CC(C)c1cc2nc(Cl)cnc2o1 ; COC(=O)c1cnc2oc(C(C)C)cc2n1]

A mixture of 2-chloro-6-isopropyl-furo[2,3-b]pyrazine (260 mg, 1.3223 mmol) and Pd(dppf)₂Cl₂CH₂Cl₂ (105 mg, 0.1286 mmol) and Et₃N (580.80 mg, 0.8 mL, 5.7397 mmol) in MeOH (15 mL) in a steel bomb equipped with mechanical stirrer was purged with carbon monoxide three times. The reaction mixture was heated to 100° C. with 120 psi of carbon monoxide and stirred for 14 h. The reaction mixture was allowed to cool to rt over 1 h. The reaction mixture was filtered through a pad of celite, washed with ethyl acetate, and the filtrate was concentrated in vacuo. The residue was purified by silica gel chromatography in 50% ethyl acetate in hexanes to give methyl 6-isopropylfuro[2,3-b]pyrazine-2-carboxylate (236 mg, 80%) as a pale yellow solid; ¹H NMR (400 MHz, CDCl₃) δ 9.02 (s, 1H), 6.72 (d, J 1.0 Hz, 1H), 4.07 (s, 3H), 3.20 (dq, J=13.7, 6.8 Hz, 1H), 1.43 (d, J=6.9 Hz, 6H); ESI-MS m/z calc. 220.0848, found 221.4 (M+1)⁺; Retention time: 2.54 minutes; LC method T.

### Step 6: 6-Isopropylfuro[2,3-b]pyrazine-2-carbaldehyde

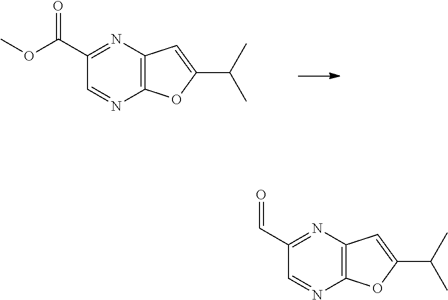 [SMILES: COC(=O)c1cnc2oc(C(C)C)cc2n1 ; CC(C)c1cc2nc(C=O)cnc2o1]

To a stirred solution of methyl 6-isopropylfuro[2,3-b]pyrazine-2-carboxylate (400 mg, 1.8163 mmol) in DCM (8 mL) was added DIBAL in DCM (3.2 mL of 1 M, 3.2000 mmol) at −78° C. over 2 minutes. The reaction mixture was stirred for 2 h, quenched with MeOH (1 mL)/water (1 mL) and concentrated in vacuo. DCM (10 mL) was added and the reaction was filtered. The cake was washed with DCM. The organic layer from the filtrate was separated, washed with brine, dried over sodium sulfate, filtered, and concentrated in vacuo. The resulting yellow oil was purified by silica gel chromatography (Silica, 12 g, loaded with DCM, eluted with 70% ethyl acetate in hexanes). The desired product fractions were combined and concentrated in vacuo to give 6-isopropylfuro[2,3-b]pyrazine-2-carbaldehyde (254 mg, 73%) as a pale yellow solid. ¹H NMR (500 MHz, DMSO) δ 10.11 (s, 1H), 8.81 (s, 1H), 7.11 (s 1H), 3.22 (dt, J 13.7, 6.9 Hz, 1H), 1.35 (d, J=6.9 Hz, 6H). ESI-MS m/z calc. 190.07423, found 191.2 (M+1)⁺; Retention time: 1.92 minutes; LC method T.

### Step 7: 3-[[4-(2,6-Dimethylphenyl)-6-[(2R)-2-[(6-isopropylfuro[2,3-b]pyrazin-2-yl)methylamino]-4,4-dimethyl-pentoxy]pyrimidin-2-yl]sulfamoyl]benzoic acid

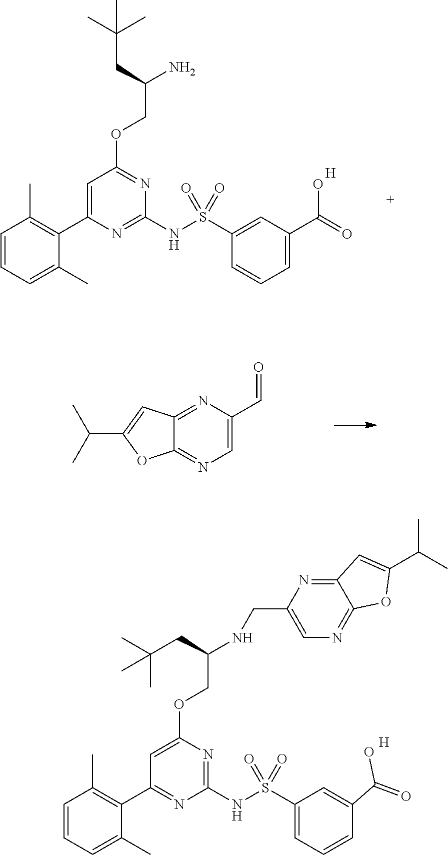

In a 4 mL vial, to a stirred mixture of 3-[[4-[(2R)-2-amino-4,4-dimethyl-pentoxy]-6-(2,6-dimethylphenyl)pyrimidin-2-yl]sulfamoyl]benzoic acid (hydrochloride salt) (45 mg, 0.08195 mmol) and 6-isopropylfuro[2,3-b]pyrazine-2-carbaldehyde (16 mg, 0.08412 mmol) in anhydrous dichloromethane (0.30 mL) were added glacial acetic acid (10 μL, 0.1758 mmol) and DIPEA (50 μL, 0.2871 mmol), in that order, at 0° C. under nitrogen. After 2-3 min, sodium triacetoxyborohydride (60 mg, 0.2831 mmol) was added to the yellow solution. The heterogeneous reaction was stirred at that temperature for 15 min. Then the reaction was quenched with aqueous 1M HCl (0.5 mL), MeOH (0.5 mL) and DMSO (0.5 mL) and purified by preparative reverse-phase HPLC (1-99% acetonitrile in water (containing 5 mM HCl) over 15 min] to furnish 3-[[4-(2,6-dimethylphenyl)-6-[(2R)-2-[(6-isopropylfuro[2,3-b]pyrazin-2-yl)methylamino]-4,4-dimethyl-pentoxy]pyrimidin-2-yl]sulfamoyl]benzoic acid (hydrochloride salt) (35 mg, 59%) as a yellow solid. ESI-MS m/z calc. 686.28864, found 687.2 (M+1)⁺; Retention time: 1.49 minutes; LC method A. Step 8: (11R)-6-(2,6-Dimethylphenyl)-11-(2,2-dimethylpropyl)-12-[(6-isopropylfuro[2,3-b]pyrazin-2-yl)methyl]-2,2-dioxo-9-oxa-2λ6-thia-3,5,12,19-tetrazatricyclo[12.3.1.14,8]nonadeca-1(18),4(19),5,7,14,16-hexaen-13-one (Compound 1333)

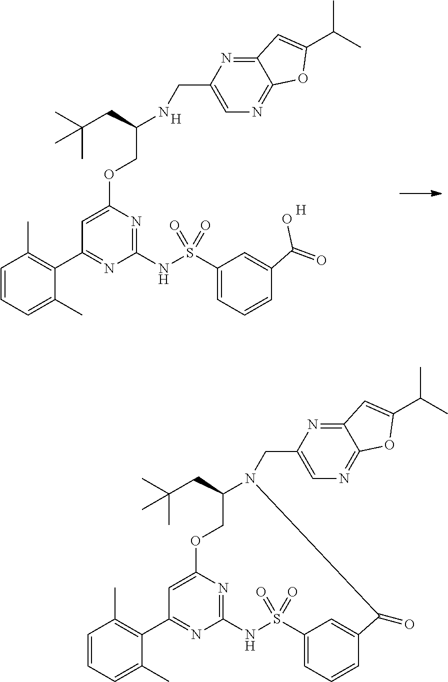

To a stirred solution of 3-[[4-(2,6-dimethylphenyl)-6-[(2R)-2-[(6-isopropylfuro[2,3-b]pyrazin-2-yl)methylamino]-4,4-dimethyl-pentoxy]pyrimidin-2-yl]sulfamoyl]benzoic acid (hydrochloride salt) (35 mg, 0.04839 mmol) in anhydrous DMF (1.7 mL) was added 2-chloro-4,6-dimethoxy-1,3,5-triazine (14 mg, 0.07974 mmol) (CDMT), followed by addition of 4-methylmorpholine (40 μL, 0.3638 mmol) at 0-4° C. (ice-water bath) under nitrogen. The yellow reaction was allowed to stir at that temperature for 5 min, then allowed to stir at room temperature for 90 min. Purification of the reaction mixture by preparative reverse-phase HPLC (1-99% acetonitrile in water over 15 min, 5 mM HCl as modifier) furnished (11R)-6-(2,6-dimethylphenyl)-11-(2,2-dimethylpropyl)-12-[(6-isopropylfuro[2,3-b]pyrazin-2-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (18 mg, 55%) as a yellow solid. ¹H NMR (400 MHz, CDCl₃) δ 8.75 (t, J 1.8 Hz, 1H), 8.40 (s, 1H), 8.03 (dt, J 8.0, 1.4 Hz, 1H), 7.82 (dt, J 7.7, 1.4 Hz, 1H), 7.60 (t, J 7.8 Hz, 1H), 7.21 (t, J 7.6 Hz, 1H), 7.04 (d, J 7.6 Hz, 2H), 6.63 (d, J 1.0 Hz, 1H), 6.21 (s, 1H), 5.45 (dd, J 10.9, 3.9 Hz, 1H), 5.26 (d, J 14.8 Hz, 1H), 4.33 (d, J 14.9 Hz, 1H), 4.26 (t, J 11.3 Hz, 1H), 4.21-4.09 (m, 1H), 3.23-3.09 (m, 1H), 1.97 (s, 6H), 1.80 (dd, J 15.2, 8.6 Hz, 1H), 1.58 (dd, J 15.1, 1.6 Hz, 1H), 1.40 (d, J 6.9 Hz, 6H), 0.64 (s, 9H). ESI-MS m/z calc. 668.2781, found 669.2 (M+1)⁺; Retention time: 2.01 minutes; LC method A.

### Example 171: Preparation of Compound 1334

### Step 1: 6-Chloro-3-(3-methylbut-1-ynyl)pyrazin-2-amine

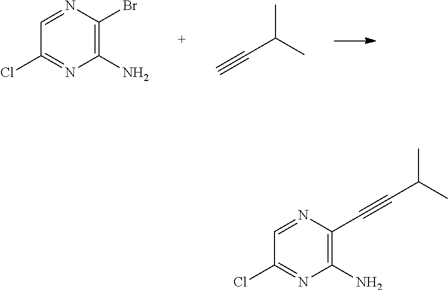

To a stirred solution of 3-bromo-6-chloro-pyrazin-2-amine (5 g, 23.987 mmol) in triethylamine (50 mL) was added 3-methylbut-1-yne (2.6640 g, 4 mL, 39.109 mmol). Nitrogen was bubbled into the mixture for 10 minutes and then bis(triphenylphosphine) palladium(II)chloride (1.7 g, 2.4220 mmol) followed by CuI (460 mg, 2.4153 mmol) was added. Nitrogen was bubbled into the mixture for 5 minutes. The mixture was stirred at room temperature for 90 minutes. The mixture was diluted by EtOAc (100 mL), was filtered on a pad of Celite and the pad was rinsed with EtOAc (100 mL). Water (100 mL) was then added, and the organic phase was washed with water (3×250 mL) and brine (1×20 mL), dried over sodium sulfate, filtered and concentrated in vacuo. The crude mixture was purified by flash-chromatography on a 120 g cartridge, eluting with a gradient of 0 to 50% of EtOAc in heptanes to afford after evaporation 6-chloro-3-(3-methylbut-1-ynyl)pyrazin-2-amine (4.5 g, 96%) as a beige solid. ¹H NMR (400 MHz, CDCl₃) δ 7.86 (s, 1H), 5.14 (br. s, 2H), 2.88 (spt, J=6.9 Hz, 1H), 1.32 (d, J=6.8 Hz, 6H). ESI-MS m/z calc. 195.05632, found 196.2 (M+1)⁺; Retention time: 1.7 minutes; LC method X.

### Step 2: 3-Chloro-6-isopropyl-5H-pyrrolo[2,3-b]pyrazine

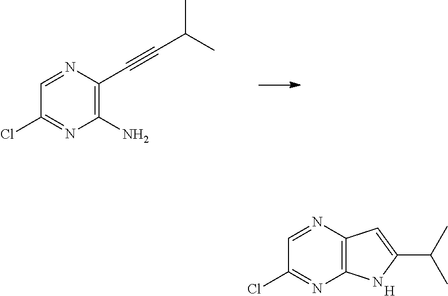

To a solution of 6-chloro-3-(3-methylbut-1-ynyl)pyrazin-2-amine (4.5 g, 22.977 mmol) in tert-butanol (50 mL) was added potassium tert-butoxide (11.5 g, 102.48 mmol). The mixture was stirred at 80° C. for 18 hours and the mixture was then cooled down to room temperature. The crude mixture was evaporated in vacuo to dryness. Water (100 mL) was then added, and the mixture was stirred at room temperature for 30 minutes. The suspension was then filtered, the solid was washed with water (100 mL) and then the solid was co-evaporated with MeCN (2×50 mL) to afford 3-chloro-6-isopropyl-5H-pyrrolo[2,3-b]pyrazine (4.45 g, 99%) as a beige solid. ¹H NMR (400 MHz, DMSO-d₆) δ 12.22 (br. s, 1H), 8.34 (s, 1H), 6.41 (s, 1H), 3.10 (spt, J 6.8 Hz, 1H), 1.32 (d, J 7.1 Hz, 6H). ESI-MS m/z calc. 195.05632, found 196.2 (M+1)⁺; Retention time: 1.64 minutes; LC method X.

### Step 3: 3-Chloro-6-isopropyl-5-methyl-pyrrolo[2,3-b]pyrazine

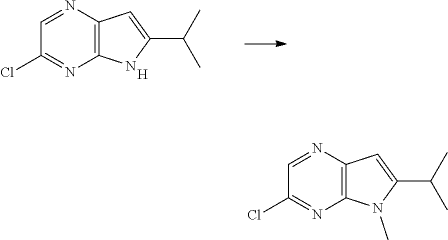

To a solution of 3-chloro-6-isopropyl-5H-pyrrolo[2,3-b]pyrazine (4.4 g, 22.467 mmol) in DMF (60 mL) at 0° C. was added NaH (in mineral dispersion) (2.5 g, 60% w/w, 62.506 mmol). The mixture was stirred at 0° C. for 5 minutes and then dimethyl sulfate (5.9850 g, 4.5 mL, 47.450 mmol) was added. The mixture was stirred at 0° C. for 5 minutes and then at room temperature for 3 hours. The mixture was then cooled down to 0° C. and water (100 mL) was added. EtOAc (250 mL) was added, and the mixture was extracted with EtOAc (3×250 mL). The combined organic layers were washed with water (3×250 mL) and brine (3×250 mL), dried over sodium sulfate, filtered, and concentrated in vacuo. The crude mixture was purified by flash-chromatography on a silica gel cartridge (120 g Gold), eluting with a gradient of 0 to 50% of EtOAc in heptane to afford after evaporation 3-chloro-6-isopropyl-5-methyl-pyrrolo[2,3-b]pyrazine (4 g, 85%) as a yellow oil. ¹H NMR (400 MHz, CDCl₃) δ 8.34 (s, 1H), 6.45 (s, 1H), 3.81 (s, 3H), 3.15 (spt, J 6.8 Hz, 1H), 1.39 (d, J 6.8 Hz, 6H). ESI-MS m/z calc. 209.07198, found 210.2 (M+1)⁺; Retention time: 1.81 minutes; LC method X.

### Step 4: Methyl 6-isopropyl-5-methyl-pyrrolo[2,3-b]pyrazine-3-carboxylate

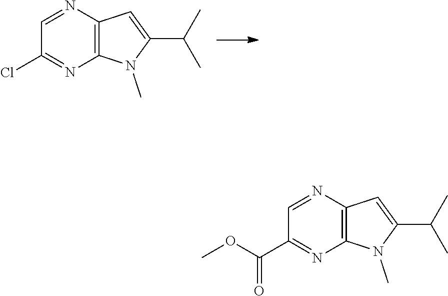 [SMILES: CC(C)c1cc2ncc(Cl)nc2n1C ; COC(=O)c1cnc2cc(C(C)C)n(C)c2n1]

To a solution of 3-chloro-6-isopropyl-5-methyl-pyrrolo[2,3-b]pyrazine (4 g, 19.058 mmol) in MeOH (20 mL) was added triethylamine (3.9930 g, 5.5 mL, 39.460 mmol). Nitrogen was bubbled into the mixture for 15 minutes and then 1,1′-Bis(diphenylphosphino)ferrocene palladium (II) chloride, complex with dichloromethane (775 mg, 0.9490 mmol) was added. Nitrogen was bubbled into the mixture for 5 minutes. The mixture was then stirred at 100° C. under 50 psi carbon monoxide pressure for 18 hours. The mixture was then cooled down to room temperature, was filtered on a Celite pad and was concentrated in vacuo. Water (100 mL) and EtOAc (100 mL) were then added, and the mixture was extracted with EtOAc (3×100 mL). The combined organic layers were washed with brine (100 mL), dried over sodium sulfate, filtered, and concentrated in vacuo. The crude mixture was purified by flash-chromatography on a 120 g cartridge, eluting with a gradient of 0 to 50% of EtOAc in heptane to afford after evaporation methyl 6-isopropyl-5-methyl-pyrrolo[2,3-b]pyrazine-3-carboxylate (4.11 g, 92%) as a beige solid. ¹H NMR (400 MHz, CDCl₃) δ 9.17 (s, 1H), 6.55 (s, 1H), 4.04 (s, 3H), 3.92 (s, 3H), 3.21 (spt, J 6.8 Hz, 1H), 1.42 (d, J 6.6 Hz, 6H). ESI-MS m/z calc. 233.11642, found 234.2 (M+1)⁺; Retention time: 1.59 minutes; LC method X.

### Step 5: (6-Isopropyl-5-methyl-pyrrolo[2,3-b]pyrazin-3-yl)methanol

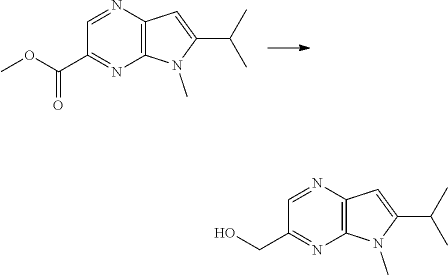 [SMILES: COC(=O)c1cnc2cc(C(C)C)n(C)c2n1 ; CC(C)c1cc2ncc(CO)nc2n1C]

To a solution of LAH (200 mg, 5.2695 mmol) in dry THF (10 mL) at 0° C. was added methyl 6-isopropyl-5-methyl-pyrrolo[2,3-b]pyrazine-3-carboxylate (1 g, 4.2827 mmol) in dry THF (10 mL). The mixture was stirred at 0° C. for 30 minutes. Water (0.2 mL) was added followed by an aqueous solution of NaOH (15%, 0.2 mL) and then by water (0.6 mL). The mixture was stirred at room temperature for 30 minutes and then magnesium sulfate (500 mg) was added. The mixture was filtered on Celite and the filter cake was washed with EtOAc (50 mL). The filtrate was then concentrated in vacuo to afford (6-isopropyl-5-methyl-pyrrolo[2,3-b]pyrazin-3-yl)methanol (750 mg, 81%) as a yellow solid.

### Step 6: 6-Isopropyl-5-methyl-pyrrolo[2,3-b]pyrazine-3-carbaldehyde

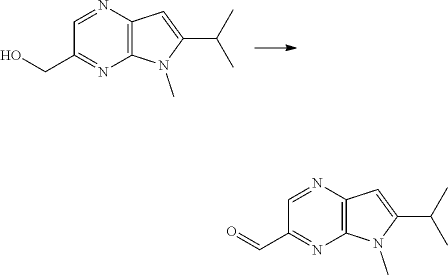

To a 0° C. solution of (6-isopropyl-5-methyl-pyrrolo[2,3-b]pyrazin-3-yl)methanol (2.5 g, 10.548 mmol) in DCM (75 mL) was added Dess-Martin periodinane (7.3 g, 17.211 mmol). The reaction was stirred at 0° C. for 5 minutes and then stirred for 2 hours at room temperature. An aqueous saturated solutions of 1N NaOH (20 mL) and water (20 mL) were added and the reaction mixture was stirred for 30 minutes. The mixture was then filtered on a silica pad and the pad was rinsed with DCM (250 mL). Water (250 mL) was added to the mixture and the aqueous one was extracted with DCM (3×250 mL). The combined organic layers were dried over sodium sulfate, filtered and concentrated in vacuo. The crude mixture was purified by flash-chromatography on a 40 g silica gel cartridge, eluting with a gradient of 0 to 100% of EtOAc in heptane to afford after evaporation 6-isopropyl-5-methyl-pyrrolo[2,3-b]pyrazine-3-carbaldehyde (2 g, 85%) as a dark orange solid. ¹H NMR (400 MHz, CDCl₃) δ 10.17 (s, 1H), 9.04 (s, 1H), 6.59 (s, 1H), 3.93 (s, 3H), 3.24 (spt, J 6.8 Hz, 1H), 1.44 (d, J 6.8 Hz, 6H). ESI-MS m/z calc. 203.10587, found 204.1 (M+1)⁺; Retention time: 3.44 minutes; LC method Y.

### Step 7: (2R)-2-Amino-3-cyclopentyl-propan-1-ol

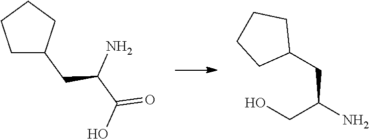 [SMILES: N[C@H](CC1CCCC1)C(=O)O ; N[C@@H](CO)CC1CCCC1]

To a stirred suspension of (2R)-2-amino-3-cyclopentyl-propanoic acid (1.00 g, 6.361 mmol) in anhydrous tetrahydrofuran (12 mL) was added borane-tetrahydrofuran (15 mL of 1.0 M, 15.00 mmol) at 0-4° C. (ice-water bath) the reaction mixture was left at room temperature for 20 h. The heterogenous mixture turned to a clear solution. Then hydrochloric acid (30 mL of 1.0 M, 30.00 mmol) was added slowly at 0-4° C. (ice-water bath). The reaction mixture was stirred for 45 min at ambient temperature. The volatiles were removed under reduced pressure. The solid residue was treated with aqueous sodium hydroxide (12.72 mL of 1.0 M, 12.72 mmol) The aqueous phase was separated and extracted with ethyl acetate (2×30 mL). The combined organic phases were washed with brine (50 mL), dried with magnesium sulfate, filtered, and concentrated under reduced pressure to obtain a semisolid. The latter was taken up in dichloromethane (20 mL) and treated with hydrogen chloride (4 M in dioxane) (5 mL of 4 M, 20.00 mmol) carefully at 0° C., and stirred for 1 h. The volatiles were removed under reduced pressure and triturated with hexanes. Then removal of the volatiles under reduced pressure and further drying furnished (2R)-2-amino-3-cyclopentyl-propan-1-ol (hydrochloride salt) (1.520 g, 133%) as a white solid. ¹H NMR (400 MHz, DMSO) δ 7.82 (s, 3H), 5.26 (t, J 5.3 Hz, 1H), 4.37 (s, 1H), 3.64-3.57 (m, 1H), 3.46-3.41 (m, 1H), 3.03 (s, 1H), 1.80-1.71 (m, 2H), 1.61-1.54 (m, 3H), 1.47-1.43 (m, 3H), 1.05 (qd, J=7.8, 3.7 Hz, 2H). ESI-MS m/z calc. 143.13101, found 142.2 (M+1)⁺; Retention time: 0.69 minutes; LC method A (1-50% gradient of MeCN).

### Step 8: 3-[[4-[(2R)-2-Amino-3-cyclopentyl-propoxy]-6-(2,6-dimethylphenyl)pyrimidin-2-yl]sulfamoyl]benzoic acid

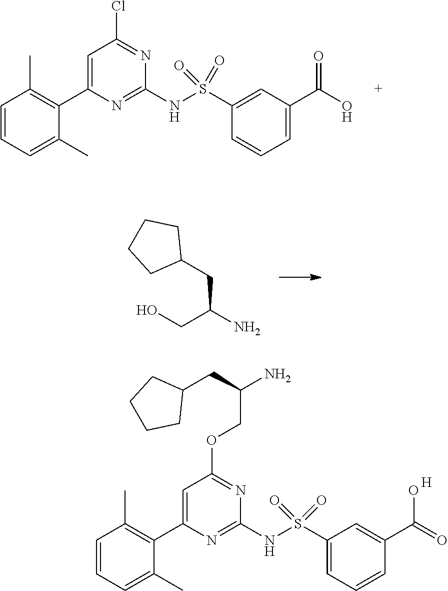

To a stirred mixture of 3-[[4-chloro-6-(2,6-dimethylphenyl)pyrimidin-2-yl]sulfamoyl]benzoic acid (1.0 g, 2.393 mmol) and (2R)-2-amino-3-cyclopentyl-propan-1-ol (hydrochloride salt) (0.55 g, 3.061 mmol) in anhydrous tetrahydrofuran (15 mL) nitrogen was purged for 5 min. Then solid sodium tert-butoxide (1.20 g, 10.08 mmol) was added at once. The heterogeneous mixture was stirred at ambient temperature for 20 h (overnight). The reaction was quenched by addition of cold hydrochloric acid (20 mL of 1 M, 20.00 mmol) and extracted with ethyl acetate (3×25 mL). The combined organics were washed with brine (20 mL), dried over sodium sulfate, filtered, and concentrated under reduced pressure. The crude was purified by preparative reverse-phase HPLC (1-99% acetonitrile in water over 15 min, 5 mM HCl as modifier) to furnish 3-[[4-[(2R)-2-amino-3-cyclopentyl-propoxy]-6-(2,6-dimethylphenyl)pyrimidin-2-yl]sulfamoyl]benzoic acid (hydrochloride salt) (387 mg, 29%) as a colorless solid. ESI-MS m/z calc. 524.20935, found 525.1 (M+1)⁺; Retention time: 1.12 minutes; LC method A.

### Step 9: 3-[[4-[(2R)-3-Cyclopentyl-2-[(6-isopropyl-5-methyl-pyrrolo[2,3-b]pyrazin-3-yl)methylamino]propoxy]-6-(2,6-dimethylphenyl)pyrimidin-2-yl]sulfamoyl]benzoic acid

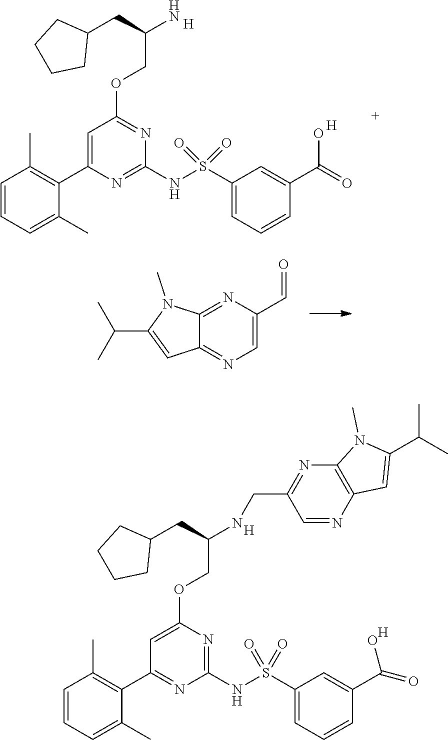

In a 4 mL vial, to a stirred mixture of 3-[[4-[(2R)-2-amino-3-cyclopentyl-propoxy]-6-(2,6-dimethylphenyl)pyrimidin-2-yl]sulfamoyl]benzoic acid (hydrochloride salt) (42 mg, 0.07485 mmol) and 6-isopropyl-5-methyl-pyrrolo[2,3-b]pyrazine-3-carbaldehyde (17 mg, 0.07612 mmol) in anhydrous dichloromethane (0.35 mL) were added glacial acetic acid (10 μL, 0.1758 mmol) and DIPEA (40 μL, 0.2296 mmol), in that order, at 0° C. under nitrogen. After 2-3 min, sodium triacetoxyborohydride (60 mg, 0.2831 mmol) was added to the yellow solution. The heterogeneous reaction was stirred at that temperature for 15 min. Then the reaction was quenched with aqueous 1M HCl (0.5 mL), MeOH (0.5 mL) and DMSO (0.5 mL) and purified by preparative reverse-phase HPLC [1-99% acetonitrile in water (containing 5 mM HCl) over 15 min] to furnish 3-[[4-[(2R)-3-cyclopentyl-2-[(6-isopropyl-5-methyl-pyrrolo[2,3-b]pyrazin-3-yl)methylamino]propoxy]-6-(2,6-dimethylphenyl)pyrimidin-2-yl]sulfamoyl]benzoic acid (hydrochloride salt) (36 mg, 64%) as a yellow solid. ESI-MS m/z calc. 711.3203, found 712.3 (M+1)⁺; Retention time: 1.5 minutes; LC method A. Step 10: (11R)-11-(Cyclopentylmethyl)-6-(2,6-dimethylphenyl)-12-[(6-isopropyl-5-methyl-pyrrolo[2,3-b]pyrazin-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (Compound 1334)

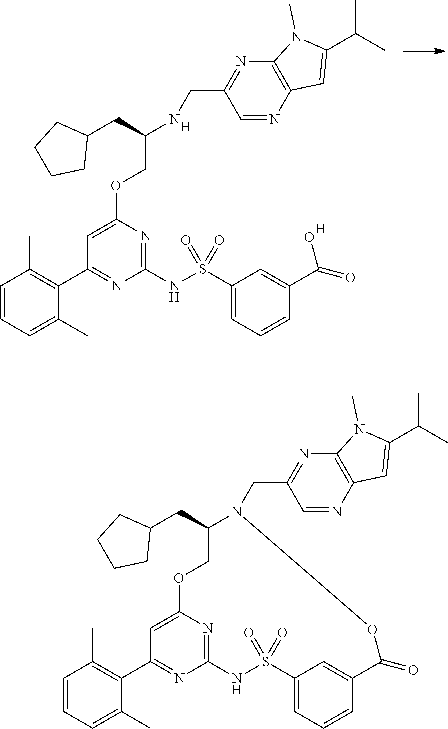

To a stirred solution of 3-[[4-[(2R)-3-cyclopentyl-2-[(6-isopropyl-5-methyl-pyrrolo[2,3-b]pyrazin-3-yl)methylamino]propoxy]-6-(2,6-dimethylphenyl)pyrimidin-2-yl]sulfamoyl]benzoic acid (hydrochloride salt) (36 mg, 0.04811 mmol) in anhydrous DMF (1.7 mL) was added CDMT (14 mg, 0.07974 mmol) (CDMT), followed by addition of 4-methylmorpholine (40 μL, 0.3638 mmol) at 0-4° C. (ice-water bath) under nitrogen. The yellow reaction was allowed to stir at that temperature for 5 min, then allowed to stir at room temperature for 90 min. Purification of the reaction mixture by preparative reverse-phase HPLC (1-99% acetonitrile in water over 15 min, 5 mM HCl as modifier) furnished (11R)-11-(cyclopentylmethyl)-6-(2,6-dimethylphenyl)-12-[(6-isopropyl-5-methyl-pyrrolo[2,3-b]pyrazin-3-yl)methyl]-2,2-dioxo-9-oxa-2λ⁶-thia-3,5,12,19-tetrazatricyclo[12.3.1.14,8]nonadeca-1(18),4(19),5,7,14,16-hexaen-13-one (18 mg, 53%) as a yellow solid. ¹H NMR (400 MHz, CDCl₃) δ 8.94 (t, J 1.8 Hz, 1H), 8.48 (s, 1H), 7.99 (d, J 7.9 Hz, 1H), 7.89-7.81 (m, 1H), 7.63 (t, J 7.8 Hz, 1H), 7.21 (t, J 7.6 Hz, 1H), 7.04 (d, J 7.6 Hz, 2H), 6.48 (s, 1H), 6.18 (s, 1H), 5.56-5.50 (m, 1H), 5.48 (d, J 15.9 Hz, 1H), 4.35 (d, J 15.8 Hz, 1H), 4.16-4.06 (m, 1H), 4.01 (t, J 11.2 Hz, 1H), 3.91 (s, 3H), 3.16 (hept, J 6.8 Hz, 1H), 2.01 (s, 6H), 1.91 (ddd, J 14.4, 10.5, 3.8 Hz, 1H), 1.79-1.68 (m, 2H), 1.63-1.47 (m, 3H), 1.47-1.41 (m, 2H), 1.40 (d, J 6.7 Hz, 3H), 1.37 (d, J 6.8 Hz, 3H), 1.33-1.25 (m, 1H), 1.14-1.03 (m, 1H), 0.58-0.46 (m, 1H). ESI-MS m/z calc. 693.30975, found 694.3 (M+1)⁺; Retention time: 1.8 minutes; LC method A. ESI-MS m/z calc. 693.30975, found 694.3 (M+1)⁺; Retention time: 1.8 minutes; LCMS Method:

### Example 172: Preparation of Compound 1335 and Compound 1336

### Step 1: Methyl 2-(tert-butoxycarbonylamino)-4-(1-methylcyclopropyl)but-2-enoate

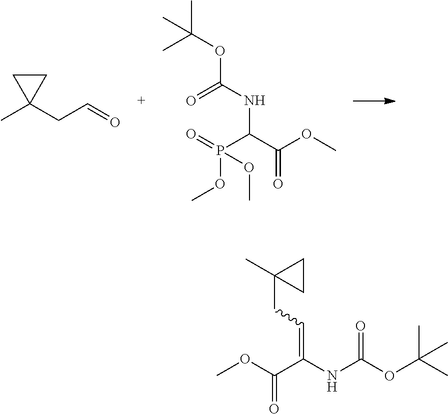

Nitrogen was bubbled to the crude solution of 2-(1-methylcyclopropyl)acetaldehyde (9.8 g, 99.854 mmol) in dichloromethane. Then methyl 2-(tert-butoxycarbonylamino)-2-dimethoxyphosphoryl-acetate (14.8 g, 49.791 mmol) was added and the mixture was cooled down to 0° C. after which time 1,8-diazabicyclo[5.4.0]undec-7-ene (38.684 g, 38 mL, 254.10 mmol) was added dropwise over 15 minutes. The reaction was stirred at 0° C. for 30 minutes then stirred overnight at room temperature. The reaction was quenched by the addition of a saturated aqueous solution of ammonium chloride (150 mL). The biphasic mixture was vigorously stirred for 30 minutes then the layers were separated, and the aqueous layer was extracted with dichloromethane (3×50 mL). The combined organic layers were washed with brine (200 mL), dried over magnesium sulfate, filtered and concentrated under reduced pressure to afford a dark brown oil which was purified by flash chromatography on silica gel (column: 120 g HP Gold; gradient: 0 to 40% ethyl acetate in heptanes, 15 CV). The desired fractions were concentrated under reduced pressure to afford methyl 2-(tert-butoxycarbonylamino)-4-(1-methylcyclopropyl)but-2-enoate (11.370 g, 81%) as a pale yellow oil which crystallized upon standing. ¹H NMR (400 MHz, CDCl₃) δ 6.68 (t, J 7.1 Hz, 1H), 5.95 (br. s, 1H), 3.79 (s, 3H), 2.14 (d, J 7.1 Hz, 2H), 1.47 (s, 9H), 1.04 (s, 3H), 0.40-0.35 (m, 2H), 0.34-0.29 (m, 2H). ESI-MS m/z calc. 269.1627, found 214.2 (M-55)⁺; Retention time: 1.87 minutes; LC method X.

### Step 2: Methyl 2-(tert-butoxycarbonylamino)-4-(1-methylcyclopropyl)butanoate

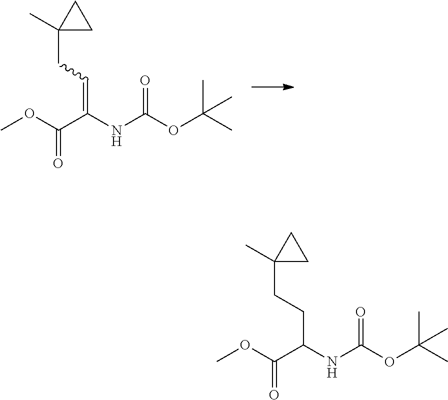 [SMILES: COC(=O)C(=CCC1(C)CC1)NC(=O)OC(C)(C)C ; COC(=O)C(CCC1(C)CC1)NC(=O)OC(C)(C)C]

To a stirred solution of methyl 2-(tert-butoxycarbonylamino)-4-(1-methylcyclopropyl)but-2-enoate (11.33 g, 39.963 mmol) in methanol (130 mL) was added nickel (II) chloride hexahydrate (9.5 g, 39.968 mmol). The green suspension was stirred for 15 minutes at room temperature until a green homogeneous solution was formed. Then a solution of sodium borohydride (15.2 g, 401.77 mmol) in methanol (130 mL) was added. The resulting black suspension was stirred at room temperature for 2 hours. The reaction was then quenched by the addition of water (200 mL). The aqueous layer was extracted with ethyl acetate (5×250 mL) and the combined organic layers were washed with water (500 mL), brine (500 mL), dried over sodium sulfate, filtered and concentrated under reduced pressure to afford crude methyl 2-(tert-butoxycarbonylamino)-4-(1-methylcyclopropyl)butanoate (10.54 g, 92%) as a colorless oil which was used in the next step without further purification. ¹H NMR (400 MHz, CDCl₃) δ 4.96 (d, J 7.1 Hz, 1H), 4.35-4.24 (m, 1H), 3.74 (s, 3H), 1.99-1.84 (m, 1H), 1.77-1.63 (m, 1H), 1.45 (s, 9H), 1.30-1.21 (m, 2H), 1.01 (s, 3H), 0.32-0.15 (m, 4H). ESI-MS m/z calc. 271.1784, found 172.2 (M-99)⁺; Retention time: 1.92 minutes; LC method X.

### Step 3: tert-Butyl N-[1-(hydroxymethyl)-3-(1-methylcyclopropyl)propyl]carbamate

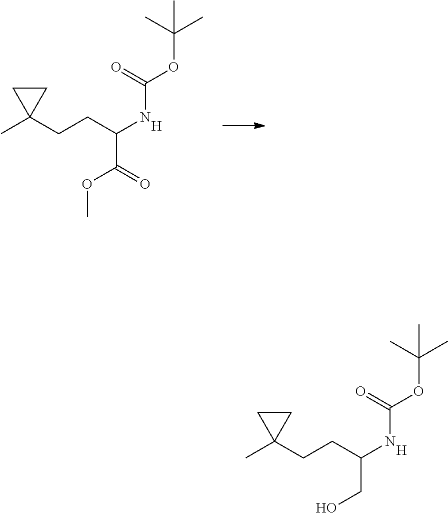

A solution of crude methyl 2-(tert-butoxycarbonylamino)-4-(1-methylcyclopropyl)butanoate (3.41 g, 11.586 mmol) was dissolved in anhydrous tetrahydrofuran (70 mL) under nitrogen atmosphere and was then cooled down to 0° C. Lithium borohydride (650 mg, 26.855 mmol) was added to the solution and the reaction was stirred at 0° C. for 15 minutes then stirred at room temperature for 2 hours. Supplementary lithium borohydride (650 mg, 26.855 mmol) was added, and the reaction was stirred at room temperature for additional 3 hours after which time another batch of lithium borohydride (650 mg, 26.855 mmol) was added. The reaction was then stirred overnight at room temperature then added to a saturated aqueous solution of ammonium chloride (250 mL). The biphasic mixture was vigorously stirred at room temperature for 30 minutes and the layers were then separated. The aqueous layer was extracted with ethyl acetate (5×50 mL) and the combined organic layers were washed with brine, dried over sodium sulfate, filtered and concentrated under reduced pressure to afford crude tert-butyl N-[1-(hydroxymethyl)-3-(1-methylcyclopropyl)propyl]carbamate (3.09 g, 101%) as a colorless oil which was directly used in the next step without further purification ¹H NMR (400 MHz, CDCl₃) δ 4.58 (br. s, 1H), 3.72-3.49 (m, 3H), 2.42 (br. s, 1H), 1.68-1.57 (m, 1H), 1.54-1.40 (m, 10H), 1.37-1.20 (m, 2H), 1.02 (s, 3H), 0.30-0.19 (m, 4H). ESI-MS m/z calc. 243.1834, found 266.2 (M+23)⁺;188.2 (M-55)⁺; Retention time: 1.75 minutes; LC method X.

### Step 4: 2-Amino-4-(1-methylcyclopropyl)butan-1-ol

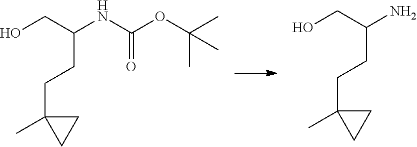

To a stirred solution of tert-butyl N-[1-(hydroxymethyl)-3-(1-methylcyclopropyl)propyl]carbamate (882 mg, 3.6245 mmol) in dichloromethane (20 mL) at 0° C. was added dropwise a solution of hydrogen chloride 4 M in dioxane (10 mL of 4 M, 40.000 mmol). The reaction was stirred for 30 minutes at 0° C. then stirred overnight at room temperature. The reaction was then concentrated under reduced pressure and the solid was co-evaporated with methanol (3×10 mL) then freeze-dried to afford 2-amino-4-(1-methylcyclopropyl)butan-1-ol (hydrochloride salt) (604 mg, 88%) as an off-white powder which could be used in the next step without further purification. ¹H NMR (400 MHz, DMSO-d₆) δ 7.89 (br. s, 3H), 5.34-5.22 (m, 1H), 3.62-3.51 (m, 1H), 3.47-3.36 (m, 1H), 3.06-2.93 (m, 1H), 1.65-1.52 (m, 2H), 1.30-1.19 (m, 2H), 0.99 (s, 3H), 0.31-0.15 (m, 4H). ESI-MS m/z calc. 143.13101, found 144.2 (M+1)⁺; Retention time: 0.81 minutes; LC method X.

### Step 5: 3-[[4-[2-Amino-4-(1-methylcyclopropyl)butoxy]-6-(2,6-dimethylphenyl)pyrimidin-2-yl]sulfamoyl]benzoic acid

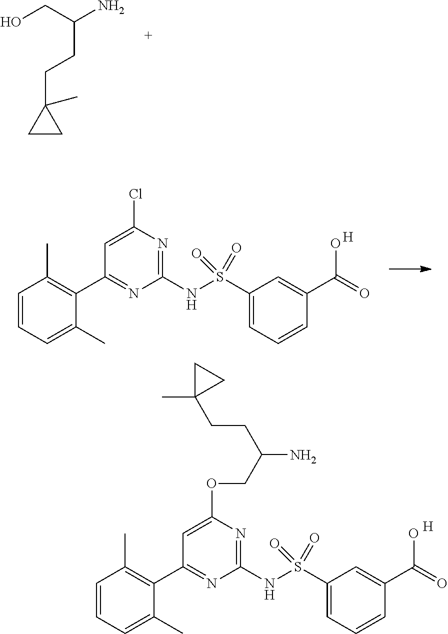

A solution of 2-amino-4-(1-methylcyclopropyl)butan-1-ol (hydrochloride salt) (604 mg, 3.3614 mmol) in anhydrous N,N-dimethylformamide (3.5 mL) was added to a solution of 3-[[4-chloro-6-(2,6-dimethylphenyl)pyrimidin-2-yl]sulfamoyl]benzoic acid (1.2 g, 2.8717 mmol) in 2-methyltetrahydrofuran (35 mL). The reaction was cooled down to 10-15° C. then sodium tert-butoxide (700 mg, 7.2838 mmol) was added. The reaction was stirred for 30 minutes at 10-15° C. then supplementary sodium tert-butoxide (700 mg, 7.2838 mmol) was added. The reaction was then stirred for 1 hour then cooled down to 0° C. and quenched by the addition of an aqueous solution of 1N hydrochloric acid (40 mL). The biphasic mixture was stirred for 30 minutes then layers were separated, and the aqueous layer was extracted with 2-methyltetrahydrofuran (5×30 mL). The combined organic layers were washed with brine (150 mL), dried over magnesium sulfate then concentrated under reduced pressure to afford a pale yellow fluffy solid which was purified by reverse-phase chromatography (column: 120 g Cis; gradient: 10 to 45% methanol in water containing 0.1% v/v of hydrochloric acid; 35 CV). The desired fractions were concentrated under reduced pressure and the residual water was co-evaporated with methanol (6×10 mL) and the residue was freeze-dried to afford 3-[[4-[2-amino-4-(1-methylcyclopropyl)butoxy]-6-(2,6-dimethylphenyl)pyrimidin-2-yl]sulfamoyl]benzoic acid (hydrochloride salt) (586 mg, 29%) as a white fluffy solid. ¹H NMR (400 MHz, DMSO-d₆) δ 13.39 (br. s, 1H), 8.49-8.41 (m, 1H), 8.16-8.03 (m, 5H), 7.69 (t, J 7.9 Hz, 1H), 7.29-7.20 (m, 1H), 7.15-7.09 (m, 2H), 6.31 (br. s, 1H), 4.38 (dd, J 12.0, 3.2 Hz, 1H), 4.25 (dd, J 11.9, 6.2 Hz, 1H), 3.55-3.48 (m, 1H, overlapped with water), 2.00 (br. s, 6H), 1.73-1.63 (m, 2H), 1.37-1.22 (m, 2H), 0.99 (s, 3H), 0.37-0.10 (m, 4H). (1H missing, labile proton). ESI-MS m/z calc. 524.20935, found 525.1 (M+1)⁺; Retention time: 2.53 minutes; LC method Y.

### Step 6: 3-[[4-[2-[[6-[Cyclobutyl(methyl)amino]pyrazin-2-yl]methylamino]-4-(1-methylcyclopropyl)butoxy]-6-(2,6-dimethylphenyl)pyrimidin-2-yl]sulfamoyl]benzoic acid

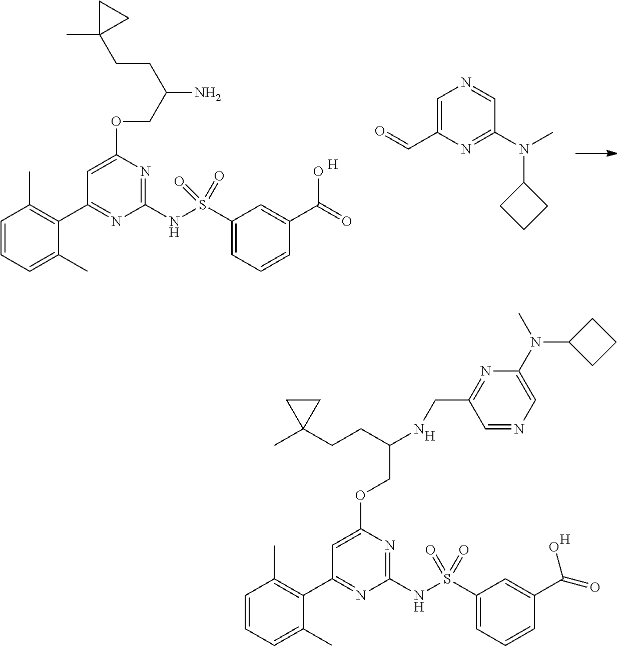

In a 4 mL vial, to a stirred mixture of 3-[[4-[2-amino-4-(1-methylcyclopropyl)butoxy]-6-(2,6-dimethylphenyl)pyrimidin-2-yl]sulfamoyl]benzoic acid (hydrochloride salt) (90 mg, 0.1464 mmol) and 6-[cyclobutyl(methyl)amino]pyrazine-2-carbaldehyde (30 mg, 0.1498 mmol) in anhydrous dichloromethane (0.60 mL) were added AcOH (20 μL, 0.3517 mmol) and DIPEA (60 μL, 0.3445 mmol), in that order, at 0-4° C. under nitrogen. After 2-3 min, sodium triacetoxyborohydride (100 mg, 0.4718 mmol) was added to the yellow solution. The heterogeneous reaction was stirred at that temperature for 15 min. Then the reaction was quenched with aqueous 1M HCl (1 mL), MeOH (1 mL) and DMSO (1 mL) and purified by preparative reverse-phase HPLC (1-99% acetonitrile in water (containing 5 mM HCl) over 15 min] to furnish 3-[[4-[2-[[6-[cyclobutyl(methyl)amino]pyrazin-2-yl]methylamino]-4-(1-methylcyclopropyl)butoxy]-6-(2,6-dimethylphenyl)pyrimidin-2-yl]sulfamoyl]benzoic acid (hydrochloride salt) (62 mg, 58%) as a yellow solid. ESI-MS m/z calc. 699.3203, found 700.3 (M+1)⁺; Retention time: 1.44 minutes; LC method A.

### Step 7: 12-[[6-[Cyclobutyl(methyl)amino]pyrazin-2-yl]methyl]-6-(2,6-dimethylphenyl)-11-[2-(1-methylcyclopropyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one, enantiomer 1 (Compound 1335) and 12-[[6-[cyclobutyl(methyl)amino]pyrazin-2-yl]methyl]-6-(2,6-dimethylphenyl)-11-[2-(1-methylcyclopropyl)ethyl]-2,2-dioxo-9-oxa-2)⁶-thia-3,5,12,19-tetrazatricyclo[12.3.1.14,8]nonadeca-1(18),4(19),5,7,14,16-hexaen-13-one, enantiomer 2 (Compounds 1336)

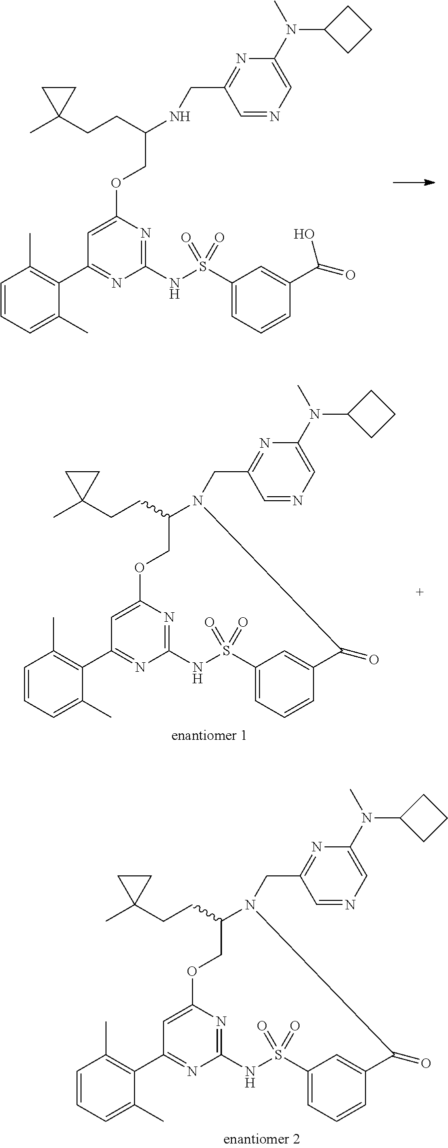 [SMILES: Cc1cccc(C)c1-c1cc(OCC(CCC2(C)CC2)NCc2cncc(N(C)C3CCC3)n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cncc(N(C)C3CCC3)n1)C(CCC1(C)CC1)CO2 ; Cc1cccc(C)c1-c1cc2nc(n1)NS(=O)(=O)c1cccc(c1)C(=O)N(Cc1cncc(N(C)C3CCC3)n1)C(CCC1(C)CC1)CO2]

To a stirred solution of 3-[[4-[2-[[6-[cyclobutyl(methyl)amino]pyrazin-2-yl]methylamino]-4-(1-methylcyclopropyl)butoxy]-6-(2,6-dimethylphenyl)pyrimidin-2-yl]sulfamoyl]benzoic acid (hydrochloride salt) (62 mg, 0.08420 mmol) in anhydrous DMF (3 mL) was added CDMT (25 mg, 0.1424 mmol), followed by addition of 4-methylmorpholine (60 μL, 0.5457 mmol) at 0-4° C. (ice-water bath) under nitrogen. The yellow reaction was allowed to stir at that temperature for 5 min, then allowed to stir at room temperature for 15 h (overnight). Purification of the reaction mixture by preparative reverse-phase HPLC (1-99% acetonitrile in water over 15 min, 5 mM HCl as modifier) furnished 12-[[6-[cyclobutyl(methyl)amino]pyrazin-2-yl]methyl]-6-(2,6-dimethylphenyl)-11-[2-(1-methylcyclopropyl)ethyl]-2,2-dioxo-9-oxa-2λ⁶-thia-3,5,12,19-tetrazatricyclo[12.3.1.14,8]nonadeca-1(18),4(19),5,7,14,16-hexaen-13-one (45 mg, 78%) as a yellow solid, ESI-MS m/z calc. 681.30975, found 6822.0 (M+1)⁺; Retention time: 1.92 minutes. The racemic mixture was subjected to chiral SFC separation (ChiralPak AD 4.6×250 mm, 5 m; Temperature: 55° C.; Mode: Isocratic; Mobile Phase: 32% MeOH+20 mM NH₃; Flow: 2.5 mL/min; Concentration: 0.4 mg/mL (in methanol:DMSO, 67:33); Injection Volume: 5 L; Pressure: 205 bar; Wavelength: 210 nm). Each separated isomer was purified by reverse-phase HPLC (1-99% acetonitrile in water over 15 min, 5 mM HCl as modifier) to give enantiomer 1, SFC Peak-1: 12-[[6-[cyclobutyl(methyl)amino]pyrazin-2-yl]methyl]-6-(2,6-dimethylphenyl)-11-[2-(1-methylcyclopropyl)ethyl]-2,2-dioxo-9-oxa-2?⁶-thia-3,5,12,19-tetrazatricyclo[12.3.1.14,8] nonadeca-1(18),4(19),5,7,14,16-hexaen-13-one (3 mg, 5%), obtained as yellow solid. ¹H NMR (400 MHz, CDCl₃) δ 8.77 (s, 1H), 8.04 (d, J 7.9 Hz, 1H), 7.90 (d, J 7.4 Hz, 2H), 7.84 (d, J 7.5 Hz, 1H), 7.65 (t, J 7.8 Hz, 1H), 7.22 (t, J 7.6 Hz, 1H), 7.06 (d, J 7.6 Hz, 2H), 6.24 (s, 1H), 5.40 (d, J 7.1 Hz, 1H), 5.14 (d, J 15.0 Hz, 1H), 4.61-4.47 (m, 1H), 4.15-3.99 (m, 3H), 3.15 (s, 3H), 2.34-2.27 (m, 2H), 2.26-2.17 (m, 2H), 2.04 (s, 6H), 1.76-1.71 (m, 4H), 1.21-1.12 (m, 1H), 0.91 (s, 3H), 0.87-0.81 (m, 1H), 0.21-0.12 (m, 2H), 0.12-0.05 (m, 2H). ESI-MS m/z calc. 681.30975, found 682.2 (M+1)⁺; Retention time: 1.87 minutes; and enantiomer 2, SFC Peak-2: 12-[[6-[cyclobutyl(methyl)amino]pyrazin-2-yl]methyl]-6-(2,6-dimethylphenyl)-11-[2-(1-methylcyclopropyl)ethyl]-2,2-dioxo-9-oxa-2⁶-thia-3,5,12,19-tetrazatricyclo [12.3.1.14,8]nonadeca-1(18),4(19),5,7,14,16-hexaen-13-one (2 mg, 3%), obtained as yellow solid. ¹H NMR (400 MHz, CDCl₃) δ 8.76 (s, 1H), 8.03 (d, J 7.9 Hz, 1H), 7.90 (d, J 7.4 Hz, 2H), 7.84 (d, J 7.6 Hz, 1H), 7.65 (t, J 7.8 Hz, 1H), 7.22 (t, J 7.6 Hz, 1H), 7.06 (d, J 7.6 Hz, 2H), 6.24 (s, 1H), 5.40 (d, J 7.0 Hz, 1H), 5.15 (d, J 14.8 Hz, 1H), 4.62-4.46 (m, 1H), 4.17-3.97 (m, 3H), 3.15 (s, 3H), 2.35-2.27 (m, 2H), 2.26-2.17 (m, 2H), 2.04 (s, 6H), 1.77-1.71 (m, 4H), 1.22-1.13 (m, 1H), 0.91 (s, 3H), 0.88-0.82 (m, 1H), 0.21-0.12 (m, 2H), 0.12-0.05 (m, 2H). ESI-MS m/z calc. 681.30975, found 682.3 (M+1)⁺; Retention time: 1.87 minutes; LC method A.

### Example 173: Preparation of Compound 1337 and Compound 1338

### Step 1: Methyl 6-[(2R)-2-methylpyrrolidin-1-yl]pyrazine-2-carboxylate

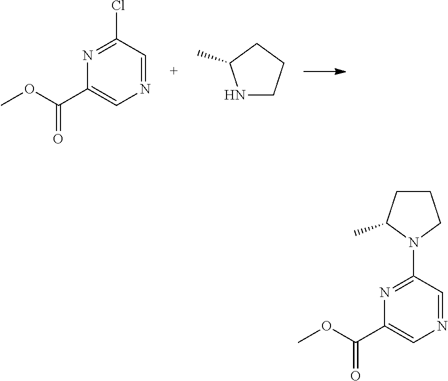

To a stirring solution of methyl 6-chloropyrazine-2-carboxylate (15 g, 85.183 mmol) and (2R)-2-methylpyrrolidine (9.1 g, 106.87 mmol) in anhydrous DMSO (250 mL) was added anhydrous sodium carbonate (18.1 g, 170.77 mmol). The resulting black mixture was stirred at room temperature overnight. The reaction mixture was poured into cold aqueous saturated ammonium chloride solution (1000 mL) and allowed to warm up to room temperature. The mixture was extracted with EtOAc (3×200 mL). The combined organic solutions were washed with brine (500 mL), dried with anhydrous sodium sulfate, and filtered. The solvent was removed under vacuum and the residue was further dried in vacuo yielding crude methyl 6-[(2R)-2-methylpyrrolidin-1-yl]pyrazine-2-carboxylate (16.58 g, 81%) as an amber oil. ESI-MS m/z calc. 221.11642, found 222.2 (M+1)⁺; Retention time: 2.31 minutes; LC method T.

### Step 2: [6-[(2R)-2-Methylpyrrolidin-1-yl]pyrazin-2-yl]methanol

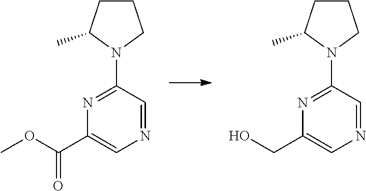 [SMILES: COC(=O)c1cncc(N2CCC[C@H]2C)n1 ; C[C@@H]1CCCN1c1cncc(CO)n1]

Methyl 6-[(2R)-2-methylpyrrolidin-1-yl]pyrazine-2-carboxylate (13.05 g, 58.981 mmol) was dissolved in MeOH (300 mL) and cooled to 0° C. using an ice bath. NaBH4 (23 g, 24.339 mL, 607.94 mmol) was then added in portions and the solution was stirred at 0° C. for 4 h and then warmed to room temperature and stirred overnight. The reaction was quenched with water (200 mL) and the resulting solution was saturated with sodium chloride and extracted with DCM (30×200 mL). The combined organic solutions were dried over anhydrous sodium sulfate, filtered, and concentrated under vacuum. The residue was further dried in vacuo yielding [6-[(2R)-2-methylpyrrolidin-1-yl]pyrazin-2-yl]methanol (9 g, 77%) as an orange oil. ESI-MS m/z calc. 193.1215, found 194.2 (M+1)⁺; Retention time: 1.3 minutes; LC method T.

### Step 3: 6-[(2R)-2-Methylpyrrolidin-1-yl]pyrazine-2-carbaldehyde

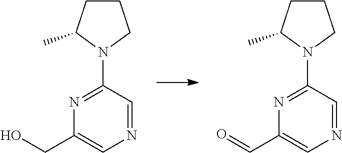

A solution of [6-[(2R)-2-methylpyrrolidin-1-yl]pyrazin-2-yl]methanol (9 g, 45.641 mmol) in anhydrous DCM (250 mL) under nitrogen was cooled to 0° C. using an ice bath. DMP (29.1 g, 68.609 mmol) was then added in portions over 5 min. The resulting amber solution was warmed up to room temperature and stirred for 5 h. The reaction mixture was filtered, and the filtrate was concentrated under reduced pressure. The crude aldehyde was purified by silica flash chromatography (330 g, dry loaded, eluting from 0 to 30% EtOAc in hexanes over a 65 min gradient). The fractions were combined and concentrated under reduced pressure and the residue was further dried in vacuo yielding 6-[(2R)-2-methylpyrrolidin-1-yl]pyrazine-2-carbaldehyde (5.2 g, 57%) as an orange liquid. ¹H NMR (500 MHz, DMSO-d₆) δ 9.89 (s, 1H), 8.23 (s, 2H), 4.32-4.21 (m, 1H), 3.65-3.56 (m, 1H), 3.45-3.36 (m, 1H), 2.12-2.02 (m, 2H), 2.02-1.93 (m, 1H), 1.75-1.69 (m, 1H), 1.20 (d, J 6.3 Hz, 3H); ESI-MS m/z calc. 191.10587, found 192.1 (M+1)⁺; Retention time: 1.65 minutes; LC method W.

### Step 4: N-Methoxy-N,3,3-trimethyl-cyclobutanecarboxamide

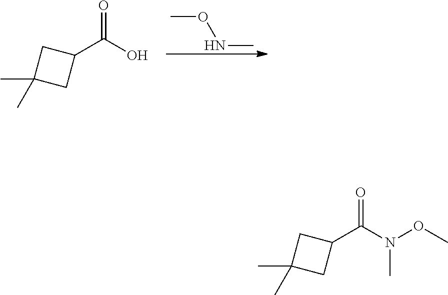

To a solution of 3,3-dimethylcyclobutanecarboxylic acid (2 g, 15.136 mmol), N-methoxymethanamine (hydrochloride salt) (3.01 g, 30.241 mmol) and HATU (6.44 g, 16.598 mmol) in DMF (30 mL) at 0° C. was added triethylamine (4.6464 g, 6.4 mL, 45.918 mmol). The reaction mixture was warmed to room temperature was stirred for 21 hours. The mixture was diluted with water (40 mL) and mixture was extracted with EtOAc (3×40 mL). The combined organic layers were washed with 1N hydrochloric acid solution (2×30 mL), saturated solution of sodium bicarbonate (2×30 mL) and brine (20 mL), dried over sodium sulfate, filtered, and concentrated under reduced pressure to afford N-methoxy-N,3,3-trimethyl-cyclobutanecarboxamide (2.72 g, 99%) as a colorless oil. ¹H NMR (400 MHz, CDCl₃) δ 3.65 (s, 3H), 3.49-3.31 (m, 1H), 3.18 (s, 3H), 2.15-2.07 (m, 2H), 1.95-1.87 (m, 2H), 1.20 (s, 3H), 1.09 (s, 3H). ESI-MS m/z calc. 171.12593, found 172.2 (M+1)⁺; Retention time: 1.67 minutes; LC method X.

### Step 5: 3,3-Dimethylcyclobutanecarbaldehyde

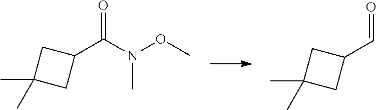

To a suspension of lithium aluminum hydride (860 mg, 22.659 mmol) in dry THF (15 mL) at 0° C. was added dropwise a solution of N-methoxy-N,3,3-trimethyl-cyclobutanecarboxamide (2.72 g, 14.931 mmol) in dry THF (15 mL). The mixture was stirred 5 min at 0° C. then allowed to reach rt and stirred for 2 h. The mixture was cooled down to 0° C., quenched with water (50 mL) and extracted with MTBE (3×50 mL). The combined organic layers were washed with brine (2×40 mL), dried over sodium sulfate, and filtered to give crude 3,3-dimethylcyclobutane carbaldehyde (1.6748 g, 100%) as a colorless solution in MTBE/THF.

### Step 6: 2-(Benzylamino)-2-(3,3-dimethylcyclobutyl)acetonitrile

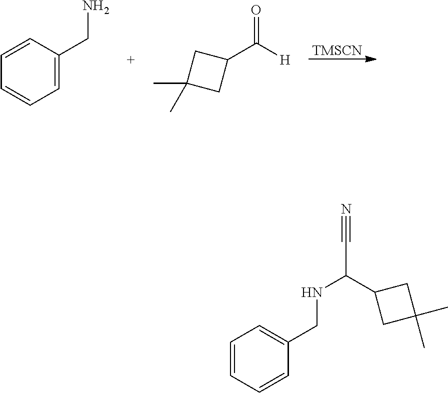

A three neck round bottom flask equipped with a 6 N NaOH trap, was charged with crude 3,3-dimethylcyclobutanecarbaldehyde (1.675 g, 14.933 mmol) as a 5:1 MTBE/THF solution (˜180 mL) and benzylamine (1.8149 g, 1.85 mL, 16.937 mmol). To the mixture was slowly added at 0° C. acetic acid (971.52 mg, 0.92 mL, 16.178 mmol) and then trimethylsilyl cyanide (1.5146 g, 1.91 mL, 15.267 mmol). The mixture was warmed to room temperature and stirred for 18 hours. The mixture was diluted with water (150 mL) and extracted with ethyl acetate (2×100 mL). The combined organic layers were washed with brine (2×60 mL), dried over anhydrous sodium sulfate, filtered, and concentrated under reduced pressure. Purification by chromatography on silica gel (Column: 120 g. Gradient: 0-20% ethyl acetate in heptanes) afforded 2-(benzylamino)-2-(3,3-dimethylcyclobutyl)acetonitrile (1.66 g, 47%) as a light yellow oil. ¹H NMR (400 MHz, CDCl₃) δ 7.38-7.27 (m, 5H), 4.07 (d, J 13.0 Hz, 1H), 3.83 (d, J 13.0 Hz, 1H), 3.46 (d, J 7.1 Hz, 1H), 2.66-2.53 (m, 1H), 1.97-1.85 (m, 2H), 1.79-1.64 (m, 2H), 1.16 (s, 3H), 1.09 (s, 3H); 1 labile proton missing. ESI-MS m/z calc. 228.16264, found 229.2 (M+1)⁺; Retention time: 1.9 minutes; LC method X.

### Step 7: 2-(Benzylamino)-2-(3,3-dimethylcyclobutyl)acetic acid

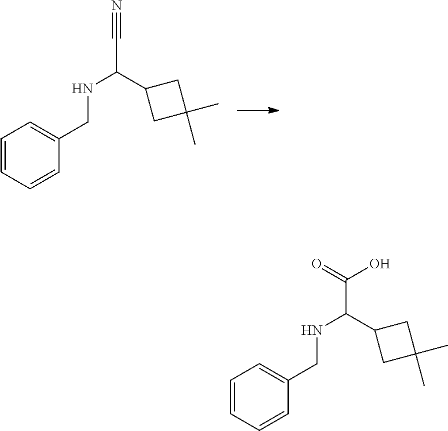 [SMILES: CC1(C)CC(C(C#N)NCc2ccccc2)C1 ; CC1(C)CC(C(NCc2ccccc2)C(=O)O)C1]

2-(Benzylamino)-2-(3,3-dimethylcyclobutyl)acetonitrile (1.66 g, 7.0229 mmol) was dissolved in acetic acid (9.8208 g, 9.3 mL, 163.54 mmol) and hydrochloric acid (56 mL of 12 M, 672.00 mmol). The mixture was stirred at 100° C. for 18 hours. More acetic acid (3.1680 g, 3 mL, 52.754 mmol) and hydrochloric acid (20 mL of 12 M, 240.00 mmol) were added, and the mixture was stirred at 100° C. for 20 hours. The solution was cooled to 10-15° C., the pH was increased to 3-4 with saturated sodium bicarbonate aqueous solution, the mixture was filtered, the solid was washed with water (15 ml) and dried under vacuum to afford 2-(benzylamino)-2-(3,3-dimethylcyclobutyl)acetic acid (1.486 g, 85%) as a grey solid. ¹H NMR (400 MHz, MeOH-d4) δ 7.52-7.40 (m, 5H), 4.19 (d, J 13.0 Hz, 1H), 4.09 (d, J 13.0 Hz, 1H), 3.38 (d, J 8.3 Hz, 1H), 2.70-2.55 (m, 1H), 1.96-1.75 (m, 4H), 1.16 (s, 3H), 1.05 (s, 3H); Two labile protons missing. ESI-MS m/z calc. 247.15723, found 248.2 (M+1)⁺; Retention time: 1.3 minutes; LC method X.

### Step 8: 2-(Benzylamino)-2-(3,3-dimethylcyclobutyl)ethanol

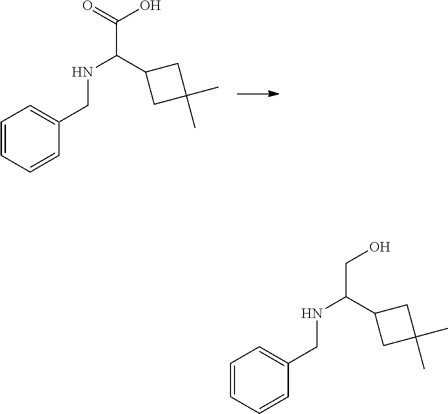 [SMILES: CC1(C)CC(C(NCc2ccccc2)C(=O)O)C1 ; CC1(C)CC(C(CO)NCc2ccccc2)C1]

To a solution of 2-(benzylamino)-2-(3,3-dimethylcyclobutyl)acetic acid (1.486 g, 6.0021 mmol) in anhydrous THF (23 mL) was added drop wise at 0° C. borane tetrahydrofuran complex solution in THF (19.4 mL of 1 M, 19.400 mmol). The reaction was stirred for 30 minutes at 0° C. and then at room temperature for 18 hours. The reaction was cooled down to 0° C. then quenched by the slow addition of methanol (25 mL) and concentrated under reduced pressure. The resulting residue was partitioned between ethyl acetate (50 mL) and 1M sodium hydroxide aqueous solution (50 mL). The biphasic mixture was vigorously stirred until complete solubilization, the layers were separated, and the aqueous layer was extracted ethyl acetate (3×40 mL). The combined organic layers were washed with brine (40 mL), dried over anhydrous sodium sulfate, filtered and concentrated under reduced pressure to afford 2-(benzylamino)-2-(3,3-dimethylcyclobutyl)ethanol (1.07 g, 69%) a colorless oil. ¹H NMR (400 MHz, CDCl₃) δ 7.45-7.28 (m, 5H), 3.83-3.73 (m, 2H), 3.59 (dd, J 10.5, 3.7 Hz, 1H), 3.27 (dd, J 10.6, 4.5 Hz, 1H), 2.60 (dt, J 9.8, 4.2 Hz, 1H), 2.36 (dq, J 17.9, 8.8 Hz, 1H), 1.92-1.80 (m, 2H), 1.54-1.43 (m, 3H), 1.14 (s, 3H), 1.04 (s, 3H); One labile proton missing. ESI-MS m/z calc. 233.17796, found 234.2 (M+1)⁺; Retention time: 1.27 minutes; LC method X.

### Step 9: 2-Amino-2-(3,3-dimethylcyclobutyl)ethanol

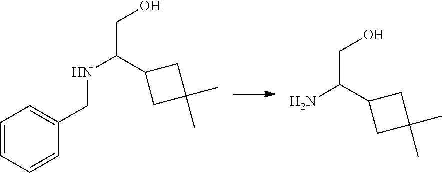

To a degassed solution of 2-(benzylamino)-2-(3,3-dimethylcyclobutyl)ethanol (1.07 g, 4.1269 mmol) in methanol (18 mL) was added 10 wt % palladium on carbon (50% wet) (455 mg, 0.2138 mmol). The mixture was purged with nitrogen for 5 minutes and then hydrogen was bubbled into the solution for 10 minutes. The mixture was then stirred under a hydrogen atmosphere (1 atm.) for 18 hours. The mixture was filtered through a short pad of Celite, the pad was rinsed with methanol (30 mL), and the filtrate was concentrated under reduced pressure to give 2-amino-2-(3,3-dimethylcyclobutyl)ethanol (632 mg, 102%) as a colorless oil. ¹H NMR (400 MHz, CDCl₃) δ 3.55 (dd, J 10.5, 3.4 Hz, 1H), 3.17 (dd, J 10.5, 7.8 Hz, 1H), 2.75-2.66 (m, 1H), 2.17-2.03 (m, 1H), 1.90-1.73 (m, 2H), 1.58-1.43 (m, 2H; overlapped with water), 1.15 (s, 3H), 1.06 (s, 3H); 3 labile protons missing. ESI-MS m/z calc. 143.13101, found 144.2 (M+1)⁺; Retention time: 0.83 minutes; LC method X.

### Step 10: 3-[[4-[2-Amino-2-(3,3-dimethylcyclobutyl)ethoxy]-6-(2,6-dimethylphenyl)pyrimidin-2-yl]sulfamoyl]benzoic acid

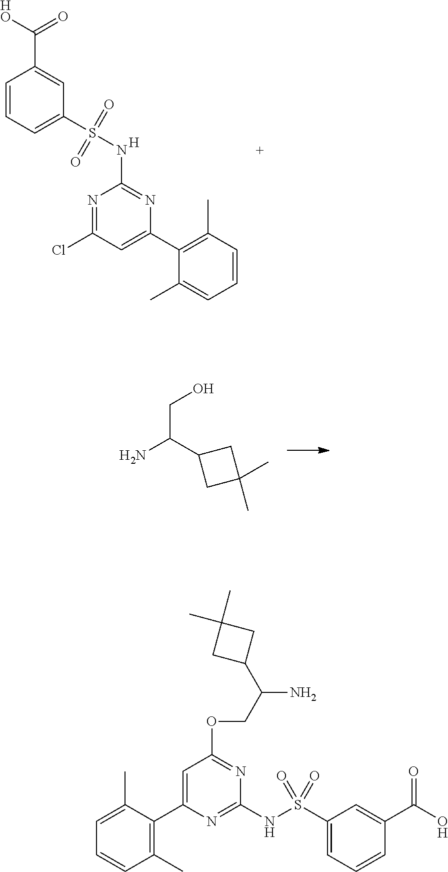

To a solution of 3-[[4-chloro-6-(2,6-dimethylphenyl)pyrimidin-2-yl]sulfamoyl]benzoic acid (1.74 g, 4.1640 mmol) and 2-amino-2-(3,3-dimethylcyclobutyl)ethanol (630 mg, 4.1787 mmol) in 2-methyltetrahydrofuran (17 mL) and N,N-dimethylformamide (1.7 mL) was added sodium tert-butoxide (1.7 g, 17.689 mmol) at 10-15° C. and then the mixture was stirred for 1 hour. The reaction was quenched with 1N hydrochloric solution (50 mL) and extracted with 2-methyltetrahydrofuran (3×50 mL). The combined organic layers were washed with brine (40 mL), dried over anhydrous sodium sulfate, filtered and concentrated under reduced pressure. The residue was triturated with ethyl acetate (80 mL), the precipitate was collected by filtration, washed with ethyl acetate (30 mL) and dried to give 3-[[4-[2-amino-2-(3,3-dimethylcyclobutyl)ethoxy]-6-(2,6-dimethylphenyl)pyrimidin-2-yl]sulfamoyl]benzoic acid (hydrochloride salt) (1.453 g, 58%) as a white solid after lyophilization. ¹H NMR (400 MHz, DMSO-d₆) δ 13.21 (br. s., 1H), 8.44 (t, J 1.6 Hz, 1H), 8.29-7.95 (m, 5H), 7.69 (t, J 7.8 Hz, 1H), 7.26 (t, J 7.6 Hz, 1H), 7.13 (d, J 7.6 Hz, 2H), 6.29 (br. s., 1H), 4.28 (dd, J 11.9, 2.8 Hz, 1H), 4.08 (dd, J 11.9, 6.5 Hz, 1H), 3.54-3.43 (m, 1H), 2.48-2.38 (m, 1H, overlapped with DMSO), 2.01 (br. s., 6H), 1.87-1.80 (m, 1H), 1.77-1.65 (m, 3H), 1.13 (s, 3H), 1.06 (s, 3H); one labile proton missing. ESI-MS m/z calc. 524.20935, found 525.1 (M+1)⁺; Retention time: 2.53 minutes; LC method Y.

### Step 11: 3-[[4-[2-(3,3-Dimethylcyclobutyl)-2-[[6-[(2R)-2-methylpyrrolidin-1-yl]pyrazin-2-yl]methylamino]ethoxy]-6-(2,6-dimethylphenyl)pyrimidin-2-yl]sulfamoyl]benzoic acid

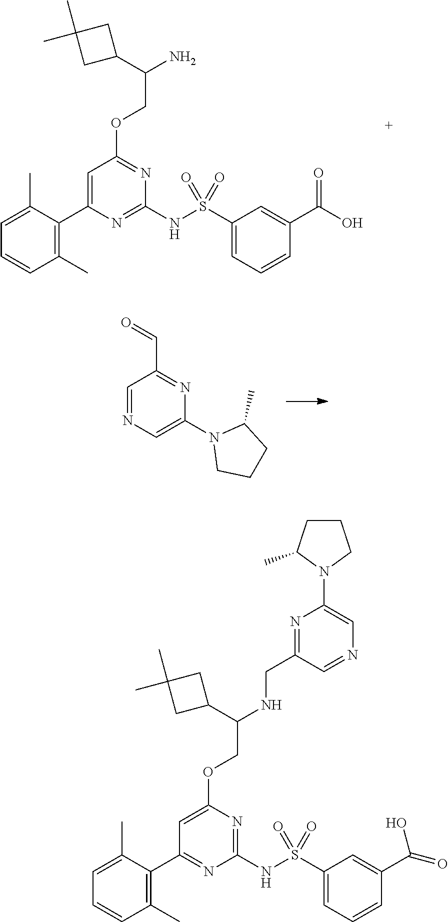 [SMILES: Cc1cccc(C)c1-c1cc(OCC(N)C2CC(C)(C)C2)nc(NS(=O)(=O)c2cccc(C(=O)O)c2)n1 ; C[C@@H]1CCCN1c1cncc(C=O)n1 ; Cc1cccc(C)c1-c1cc(OCC(NCc2cncc(N3CCC[C@H]3C)n2)C2CC(C)(C)C2)nc(NS(=O)(=O)c2cccc(C(=O)O)c2)n1]

A 20 mL vial was charged under nitrogen with 3-[[4-[2-amino-2-(3,3-dimethylcyclobutyl)ethoxy]-6-(2,6-dimethylphenyl)pyrimidin-2-yl]sulfamoyl]benzoic acid (hydrochloride salt) (258.2 mg, 0.4602 mmol), 6-[(2R)-2-methylpyrrolidin-1-yl]pyrazine-2-carbaldehyde (100.2 mg, 0.5240 mmol), anhydrous DCM (2.4 mL), and acetic acid (0.04 mL, 0.7034 mmol). The mixture was cooled down in an ice bath. DIEA (0.18 mL, 1.033 mmol) was added, followed by sodium triacetoxyborohydride (819 mg, 3.864 mmol), and the reaction was vigorously stirred at 0° C. for 1 h. The reaction was quenched with 3 N aqueous HCl, diluted with MeOH and DMSO, and the resulting solution was filtered. Purification by reverse phase HPLC (1-99% acetonitrile/5 mM aqueous HCl over 15 min) provided 3-[[4-[2-(3,3-dimethylcyclobutyl)-2-[[6-[(2R)-2-methylpyrrolidin-1-yl]pyrazin-2-yl]methylamino]ethoxy]-6-(2,6-dimethylphenyl)pyrimidin-2-yl]sulfamoyl]benzoic acid (hydrochloride salt) (163.3 mg, 47%) as a yellow solid. ESI-MS m/z calc. 699.3203, found 700.2 (M+1)⁺; Retention time: 1.39 minutes; LC method A.

### Step 12: 11-(3,3-Dimethylcyclobutyl)-6-(2,6-dimethylphenyl)-12-[[6-[(2R)-2-methylpyrrolidin-1-yl]pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 1337) and 11-(3,3-dimethylcyclobutyl)-6-(2,6-dimethylphenyl)-12-[[6-[(2R)-2-methylpyrrolidin-1-yl]pyrazin-2-yl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 2 (Compound 1338)

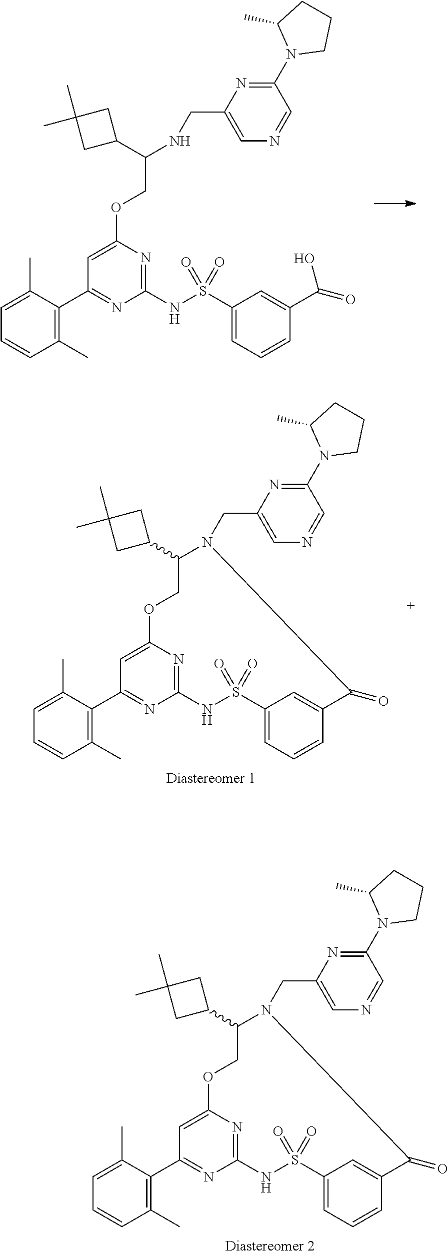 [SMILES: Cc1cccc(C)c1-c1cc(OCC(NCc2cncc(N3CCC[C@H]3C)n2)C2CC(C)(C)C2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cncc(N3CCC[C@H]3C)n1)C(C1CC(C)(C)C1)CO2 ; Cc1cccc(C)c1-c1cc2nc(n1)NS(=O)(=O)c1cccc(c1)C(=O)N(Cc1cncc(N3CCC[C@H]3C)n1)C(C1CC(C)(C)C1)CO2]

3-[[4-[2-(3,3-Dimethylcyclobutyl)-2-[[6-[(2R)-2-methylpyrrolidin-1-yl]pyrazin-2-yl]methylamino]ethoxy]-6-(2,6-dimethylphenyl)pyrimidin-2-yl]sulfamoyl]benzoic acid (hydrochloride salt) (163.3 mg, 0.2151 mmol) was combined with CDMT (48.4 mg, 0.2757 mmol) in DMF (17 mL) and cooled to 0° C. N-methylmorpholine (0.15 mL, 1.364 mmol) was added by syringe and the reaction was stirred at 0° C. for 30 minutes. The ice bath was then removed and stirring was continued for an additional 16 hours at room temperature. The reaction mixture was then partitioned between 130 mL 1M HCl and 130 mL ethyl acetate. The layers were separated, and the aqueous layer was extracted with an additional 130 mL ethyl acetate. The combined organic layers were washed 2×130 mL with brine, dried over sodium sulfate, filtered and concentrated. The crude product was purified by reverse phase HPLC (1-99% acetonitrile/5 mM aqueous HCl over 25 min) to give 82.6 mg of a diastereomeric mixture. This material was subjected to chiral SFC separation using a ChiralPak AS (21.2×250 mm, 5 m) at 40° C. The mobile phase was 46% MeOH (20 mM NH₃) at 70 mL/min flow in isocratic mode. Concentration of the sample was 27.5 mg/mL in MeOH. The injection volume was 500 μL with an outlet pressure of 191 bar and the detection wavelength was 210 nm. For each isomer, the solvents were evaporated. The product was dissolved in DMSO (1 mL) and purified by reverse phase HPLC (1-99% acetonitrile/5 mM aqueous HCl over 20 mins) to give as white solids: diastereomer 1, SFC peak 1, 11-(3,3-dimethylcyclobutyl)-6-(2,6-dimethylphenyl)-12-[[6-[(2R)-2-methylpyrrolidin-1-yl]pyrazin-2-yl]methyl]-2,2-dioxo-9-oxa-2?⁶-thia-3,5,12,19-tetrazatricyclo[12.3.1.14,8]nonadeca-1(18),4(19),5,7,14,16-hexaen-13-one (25.2 mg, 17%), ¹H NMR (400 MHz, CDCl₃) δ 8.81 (s, 1H), 8.13-8.09 (m, 1H), 7.91-7.86 (m, 1H), 7.83 (s, 1H), 7.74 (s, 1H), 7.68 (t, J 7.8 Hz, 1H), 7.20 (t, J 7.6 Hz, 1H), 7.05 (d, J 7.6 Hz, 2H), 6.11 (s, 1H), 5.51 (dd, J 11.3, 3.9 Hz, 1H), 5.09 (d, J 15.8 Hz, 1H), 4.28-4.20 (m, 1H), 4.12 (t, J 11.4 Hz, 1H), 4.02-3.94 (m, 2H), 3.70-3.63 (m, 1H), 3.50-3.39 (m, 1H), 2.74-2.60 (m, 1H), 2.18-2.03 (m, 4H), 2.01 (s, 6H), 1.87-1.71 (m, 6H), 1.11 (s, 3H), 0.89-0.81 (m, 1H), 0.77 (s, 3H). ESI-MS m/z calc. 681.30975, found 682.2 (M+1)⁺; Retention time: 1.795 minutes, and diastereomer 2, SFC peak 2, 11-(3,3-dimethylcyclobutyl)-6-(2,6-dimethylphenyl)-12-[[6-[(2R)-2-methylpyrrolidin-1-yl]pyrazin-2-yl]methyl]-2,2-dioxo-9-oxa-2?⁶-thia-3,5,12,19-tetrazatricyclo[12.3.1.14,8]nonadeca-1(18),4(19),5,7,14,16-hexaen-13-one (26.5 mg, 18%), ¹H NMR (400 MHz, CDCl₃) δ 8.76 (s, 1H), 8.11-8.06 (m, 1H), 7.90-7.85 (m, 1H), 7.83 (s, 1H), 7.76 (s, 1H), 7.67 (t, J 7.8 Hz, 1H), 7.20 (t, J 7.6 Hz, 1H), 7.05 (d, J 7.6 Hz, 2H), 6.09 (s, 1H), 5.50 (dd, J=11.4, 4.0 Hz, 1H), 5.06 (d, J 15.5 Hz, 1H), 4.24-4.14 (m, 2H), 4.01-3.91 (m, 2H), 3.67-3.60 (m, 1H), 3.51-3.42 (m, 2H), 2.76-2.63 (m, 1H), 2.13-2.05 (m, 4H), 2.01 (s, 6H), 1.27 (d, J 6.3 Hz, 3H), 1.19 (t, J 10.2 Hz, 2H), 1.11 (s, 3H), 0.89-0.82 (m, 1H), 0.77 (s, 3H). ESI-MS m/z calc. 681.30975, found 682.3 (M+1)⁺; Retention time: 1.805 minutes; LC method A.

### Example 174: Preparation of Compound 1339 and Compound 1340

### Step 1: 2-Chloro-6-(dimethoxymethyl)pyrazine

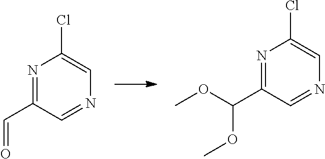

In a round bottom flask, 6-chloropyrazine-2-carbaldehyde (9 g, 63.1 mmol) was combined with DCM (5 mL) and MeOH (1 mL). Purification by flash chromatography on silica gel (0-10% methanol/dichloromethane over 35 minutes) gave 2-chloro-6-(dimethoxymethyl)pyrazine (5.02 g, 76%) as a golden yellow oil. ESI-MS m/z calc. 188.03525, found 189.0 (M+1)⁺; Retention time: 0.76 minutes; LC method A.

### Step 2: Methyl (E)-2-(tert-butoxycarbonylamino)-3-(3,3-dimethylcyclobutyl)prop-2-enoate

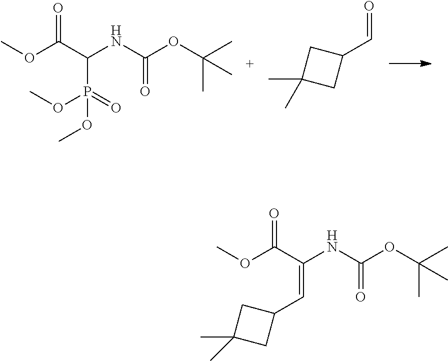

In a flame-dried flask under N₂ was placed 3,3-dimethylcyclobutanecarbaldehyde (1.6695 g, 14.884 mmol) (as solution in diethyl ether/THF), methyl 2-(tert-butoxycarbonylamino)-2-dimethoxyphosphoryl-acetate (1.806 g, 5.9543 mmol) and dry dioxane (119 mL). The solution was cooled to 0° C. and 1,1,3,3-tetramethylguanidine (3.5086 g, 3.9 mL, 29.853 mmol) was added dropwise. Reaction was slowly allowed to reach rt and stirred for 3 days. Water was added (150 mL) and the mixture was extracted with EtOAc (3×60 mL). Organic phase was washed with brine (50 mL), dried over sodium sulfate, filtered and concentrated under reduced pressure. Residue was purified by silica gel chromatography on a 25 g GOLD cartridge, eluting with a gradient of AcOEt in heptanes (0 to 15% over 18 CV). Desired fractions were combined and concentrated under reduced pressure to afford methyl (E)-2-(tert-butoxycarbonylamino)-3-(3,3-dimethylcyclobutyl)prop-2-enoate (1.817 g, 99%) as a white semi-solid. ¹H NMR (400 MHz, CDCl₃) δ 6.62 (d, J 8.1 Hz, 1H), 5.86 (br. s., 1H), 3.78 (s, 3H), 3.20 (sxt, J 8.6 Hz, 1H), 2.07-2.01 (m, 2H), 1.76-1.70 (m, 2H), 1.47 (s, 9H), 1.17 (s, 3H), 1.07 (s, 3H). ESI-MS m/z calc. 283.1784, found 306.2 (M+23)⁺;228.2 (M-55)⁺; Retention time: 1.948 minutes; LC method X.

### Step 3: Methyl 2-(tert-butoxycarbonylamino)-3-(3,3-dimethylcyclobutyl)propanoate

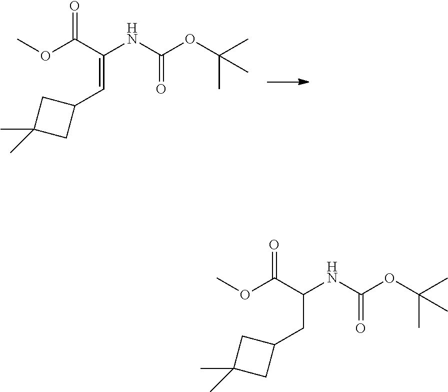 [SMILES: COC(=O)/C(=C\C1CC(C)(C)C1)NC(=O)OC(C)(C)C ; COC(=O)C(CC1CC(C)(C)C1)NC(=O)OC(C)(C)C]

In a flame-dried flask under N₂ was placed methyl (E)-2-(tert-butoxycarbonylamino)-3-(3,3-dimethylcyclobutyl)prop-2-enoate (1.817 g, 5.8993 mmol), palladium on carbon (628 mg, 10% w/w, 0.5901 mmol) and MeOH (23 mL). Hydrogen was bubbled into the suspension for 5 minutes. Reaction was then stirred under H2 (1 atm.) for 19 h. Nitrogen was then bubbled into the mixture for 10 minutes. The reaction mixture was directly filtered over a pad of Celite with MeOH. Solution was filtered again over a pad of Celite then through a nylon 0.45 m filter to obtain a clear solution. The solvent was removed under reduced pressure to afford methyl 2-(tert-butoxycarbonylamino)-3-(3,3-dimethylcyclobutyl)propanoate (1.6016 g, 90%) as a pale yellow oil. ¹H NMR (400 MHz, CDCl₃) δ 4.94 (d, J 7.3 Hz, 1H), 4.28-4.16 (m, 1H), 3.73 (s, 3H), 2.27 (spt, J 8.1 Hz, 1H), 1.93-1.82 (m, 3H), 1.78-1.71 (m, 1H), 1.52-1.36 (m, 11H), 1.12 (s, 3H), 1.04 (s, 3H). ESI-MS m/z calc. 285.194, found 308.2 (M+23)⁺;186.2 (M-99)⁺; Retention time: 1.999 minutes; LC method X.

### Step 4: tert-Butyl N-[1-[(3,3-dimethylcyclobutyl)methyl]-2-hydroxy-ethyl]carbamate

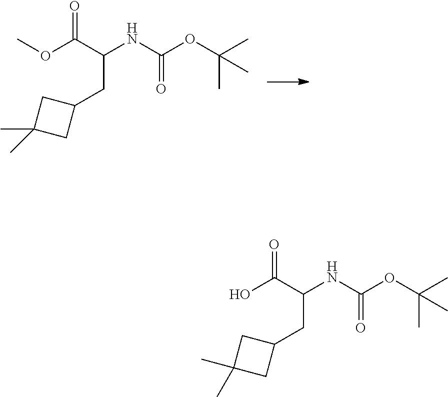 [SMILES: COC(=O)C(CC1CC(C)(C)C1)NC(=O)OC(C)(C)C ; CC1(C)CC(CC(NC(=O)OC(C)(C)C)C(=O)O)C1]

In a flame-dried flask under N₂ was placed methyl 2-(tert-butoxycarbonylamino)-3-(3,3-dimethylcyclobutyl)propanoate (1.6 g, 5.3263 mmol) and dry THF (20 mL). The resulting solution was cooled down to 0° C. and a solution of LiAlH₄ in THF (4.2 mL of 2 M, 8.4000 mmol) was added dropwise. The mixture was stirred 5 min at 0° C. then warmed to rt and stirred for 2 h. Mixture was cooled down to 0° C. Water (319 μL) was added dropwise followed by NaOH 15% aqueous sol. (319 μL) then water (957 μL). Solution was allowed to reach rt and stirred for 30 min. Magnesium sulfate was then added to the mixture and stirring was continued for 15 min. Solution was directly filtered over a pad of Celite, washed with DCM and concentrated under reduced pressure to afford crude tert-butyl N-[1-[(3,3-dimethylcyclobutyl)methyl]-2-hydroxy-ethyl]carbamate (1.26 g, 83%) as a white solid. ¹H NMR (400 MHz, CDCl₃) δ 4.55 (br. s., 1H), 3.68-3.61 (m, 1H), 3.60-3.53 (m, 1H), 3.53-3.46 (m, 1H), 2.36 (br. s, 1H), 2.31-2.20 (m, 1H), 1.94-1.85 (m, 2H), 1.56-1.49 (m, 2H, overlapped with water), 1.49-1.37 (m, 11H), 1.13 (s, 3H), 1.04 (s, 3H). ESI-MS m/z calc. 257.1991, found 280.2 (M+23)⁺;202.2 (M-55)⁺; Retention time: 1.842 minutes; LC method X.

### Step 5: 2-Amino-3-(3,3-dimethylcyclobutyl)propan-1-ol

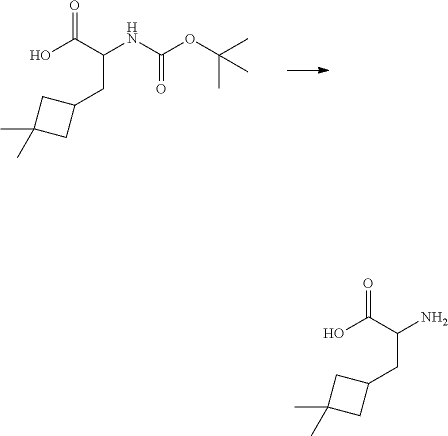 [SMILES: CC1(C)CC(CC(NC(=O)OC(C)(C)C)C(=O)O)C1 ; CC1(C)CC(CC(N)C(=O)O)C1]

In a reaction tube under N₂ was placed tert-butyl N-[1-[(3,3-dimethylcyclobutyl)methyl]-2-hydroxy-ethyl]carbamate (1.26 g, 4.6509 mmol) and dry DCM (18 mL). The resulting solution was cooled down to 0° C. and a solution of HCl in dioxane (11.6 mL of 4 M, 46.400 mmol) was added dropwise. The mixture was stirred 5 min at 0° C. then warmed to rt and stirred 16 h. Solution was concentrated under reduced pressure to afford crude 2-amino-3-(3,3-dimethylcyclobutyl)propan-1-ol (hydrochloride salt) (992.1 mg, 105%) as a light pink solid. ¹H NMR (400 MHz, DMSO-d₆) δ 8.05-7.57 (m, 3H), 5.34-5.17 (m, 1H), 3.58-3.50 (m, 1H), 2.91 (br. s, 1H), 2.35-2.23 (m, 1H), 1.89-1.79 (m, 2H), 1.64-1.55 (m, 2H), 1.44-1.34 (m, 2H), 1.11 (s, 3H), 1.03 (s, 3H). (1H missing, labile proton) ESI-MS m/z calc. 157.14667, found 158.2 (M+1)⁺; Retention time: 1.158 minutes; LC method X.

### Step 6: 3-[[4-[2-Amino-3-(3,3-dimethylcyclobutyl)propoxy]-6-dimethylphenyl)pyrimidin-2-yl]sulfamoyl]benzoic acid

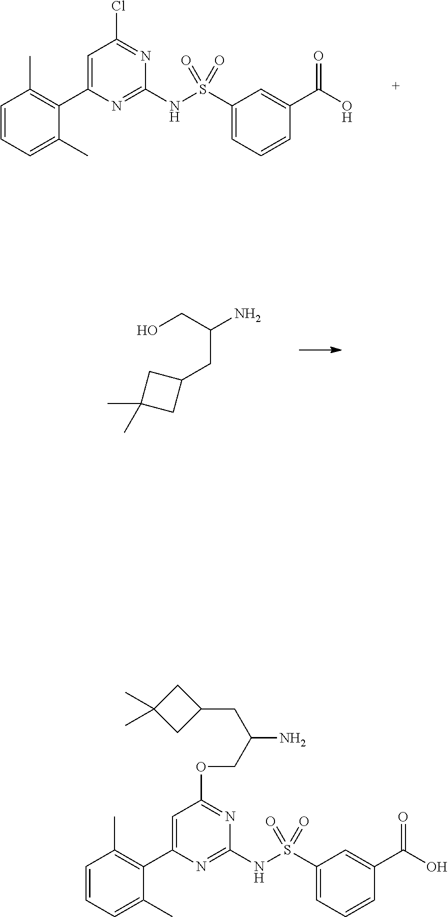 [SMILES: Cc1cccc(C)c1-c1cc(Cl)nc(NS(=O)(=O)c2cccc(C(=O)O)c2)n1 ; CC1(C)CC(CC(N)CO)C1 ; Cc1cccc(C)c1-c1cc(OCC(N)CC2CC(C)(C)C2)nc(NS(=O)(=O)c2cccc(C(=O)O)c2)n1]

A solution of 2-amino-3-(3,3-dimethylcyclobutyl)propan-1-ol (hydrochloride salt) (990 mg, 4.8551 mmol) in dry DMF (3.3 mL) was added to a solution of 3-[[4-chloro-6-(2,6-dimethylphenyl)pyrimidin-2-yl]sulfamoyl]benzoic acid (2.13 g, 5.0973 mmol) in 2-MeTHF (29.7 mL). The mixture was then cooled down to 10-15° C. and sodium tert-butoxide (2.8 g, 28.261 mmol) was added. The reaction was stirred at 10-15° C. for 2 h then cooled down to 0° C. and quenched by the addition of an aqueous solution of 1N HCl (50 mL). The biphasic mixture was stirred for 15 minutes at 0° C. then concentrated under reduced pressure. Residue was purified by reverse phase chromatography on a 120 g C₁₈ GOLD cartridge, eluting with a gradient of MeCN in acidic water (0.1% v/v of HCl in water) (5% for 4 CV then 5 to 70% over 20 CV). Desired fractions were combined and concentrated under reduced pressure. Product was then lyophilized to afford 3-[[4-[2-amino-3-(3,3-dimethylcyclobutyl)propoxy]-6-(2,6-dimethylphenyl)pyrimidin-2-yl]sulfamoyl]benzoic acid (hydrochloride salt) (2.1251 g, 74%) as an off-white solid. ¹H NMR (400 MHz, DMSO-d₆) δ 13.81-11.71 (m, 1H), 8.48-8.42 (m, 1H), 8.23-8.05 (m, 5H), 7.71 (t, J 7.8 Hz, 1H), 7.26 (t, J 7.6 Hz, 1H), 7.13 (d, J 7.6 Hz, 2H), 6.30 (br. s., 1H), 4.31 (dd, J 11.7, 2.9 Hz, 1H), 4.09 (dd, J 11.7, 6.1 Hz, 1H), 3.40-3.33 (m, 1H, overlapped with water), 2.35-2.23 (m, 1H), 2.10-1.93 (m, 6H, overlapped with MeCN), 1.86 (dd, J 10.9, 7.9 Hz, 1H), 1.81-1.65 (m, 3H), 1.44 (t, J 9.8 Hz, 1H), 1.37 (t, J 9.8 Hz, 1H), 1.10 (s, 3H), 1.02 (s, 3H). (1H missing, labile proton) ESI-MS m/z calc. 538.225, found 539.2 (M+1)⁺; Retention time: 2.642 minutes; LC method X.

### Step 7: 3-[[4-[2-[(6-Chloropyrazin-2-yl)methylamino]-3-(3,3-dimethylcyclobutyl)propoxy]-6-(2,6-dimethylphenyl)pyrimidin-2-yl]sulfamoyl]benzoic acid

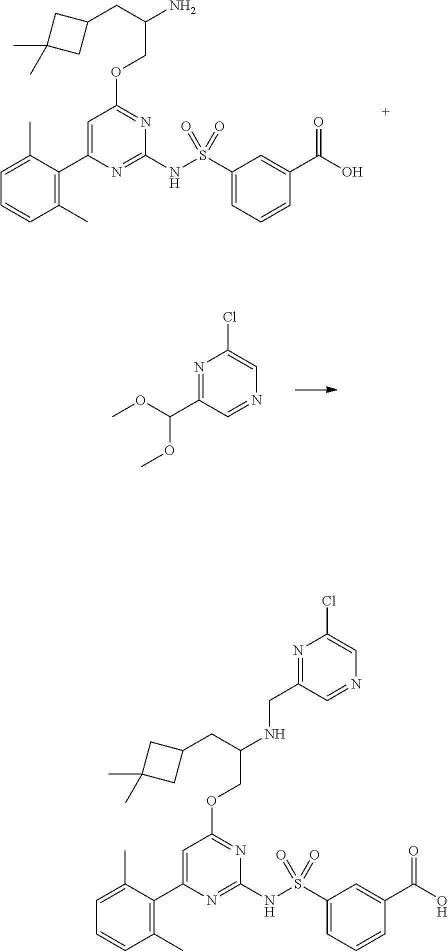 [SMILES: Cc1cccc(C)c1-c1cc(OCC(N)CC2CC(C)(C)C2)nc(NS(=O)(=O)c2cccc(C(=O)O)c2)n1 ; COC(OC)c1cncc(Cl)n1 ; Cc1cccc(C)c1-c1cc(OCC(CC2CC(C)(C)C2)NCc2cncc(Cl)n2)nc(NS(=O)(=O)c2cccc(C(=O)O)c2)n1]

In a 20 mL scintillation vial, 3-[[4-[2-amino-3-(3,3-dimethylcyclobutyl)propoxy]-6-(2,6-dimethylphenyl)pyrimidin-2-yl]sulfamoyl]benzoic acid (hydrochloride salt) (500 mg, 0.8694 mmol) as added to a solution of 2-chloro-6-(dimethoxymethyl)pyrazine (177 mg, 0.9384 mmol) in DCM (5 mL). Glacial acetic acid (75 μL, 1.319 mmol) was added. After brief stirring, DIEA (365 μL, 2.096 mmol) was added at 0° C. Stirring was continued for 15 minutes before the addition of sodium triacetoxyborohydride (553 mg, 2.609 mmol). The reaction mixture was stirred at 0° C. for 30 minutes. The reaction mixture was quenched with the addition of aqueous 1 M HCl. After stirring for 10 minutes, it was taken up in EtOAc (50 mL) and washed with aqueous HCl (1×50 mL) and brine (1×50 mL). The organic layer was dried over sodium sulfate, filtered and concentrated under reduced pressure. The crude product was triturated with EtOAc and hexanes. Solids were collected by vacuum filtration, rinsing with hexanes, to provide 3-[[4-[2-[(6-chloropyrazin-2-yl)methylamino]-3-(3,3-dimethylcyclobutyl)propoxy]-6-(2,6-dimethylphenyl)pyrimidin-2-yl]sulfamoyl]benzoic acid (hydrochloride salt) (609 mg, 100%) as an off-white solid. ESI-MS m/z calc. 664.22345, found 665.2 (M+1)⁺; Retention time: 1.25 minutes; LC method A.

### Step 8: 12-[(6-Chloropyrazin-2-yl)methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one

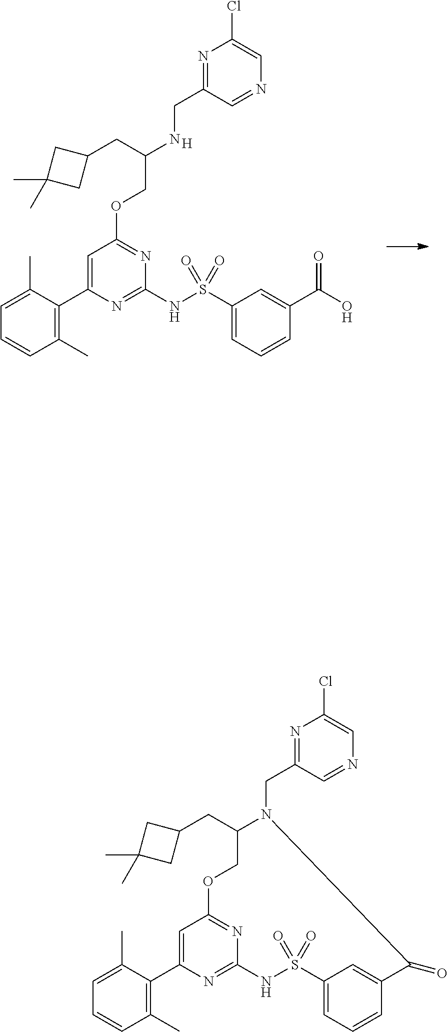

In a round bottom flask, 3-[[4-[2-[(6-chloropyrazin-2-yl)methylamino]-3-(3,3-dimethylcyclobutyl)propoxy]-6-(2,6-dimethylphenyl)pyrimidin-2-yl]sulfamoyl]benzoic acid (hydrochloride salt) (610 mg, 0.8694 mmol) was combined with 2-chloro-4,6-dimethoxy-1,3,5-triazine (198 mg, 1.128 mmol). The mixture was dissolved in DMF (6 mL) and cooled to 0° C. before the addition of 4-methylmorpholine (480 μL, 4.366 mmol). The reaction mixture was allowed to slowly warm to room temperature and stirred overnight. It was diluted with EtOAc (75 mL) and washed with aqueous HCl (1 M, 1×75 mL) and brine (1×75 mL). The organic layer was dried over sodium sulfate, filtered, and concentrated under reduced pressure. Purification by flash chromatography on silica gel (0-50% EtOAc/hexanes over 20 minutes) gave 12-[(6-chloropyrazin-2-yl)methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (294 mg, 52%) as a white solid. ESI-MS m/z calc. 646.2129, found 647.3 (M+1)⁺; Retention time: 1.86 minutes; LC method A.

### Step 9: 12-[[6-[Cyclobutyl(methyl)amino]pyrazin-2-yl]methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 1339) and 12-[[6-[cyclobutyl(methyl)amino]pyrazin-2-yl]methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one, diastereomer 1 (Compound 1340)

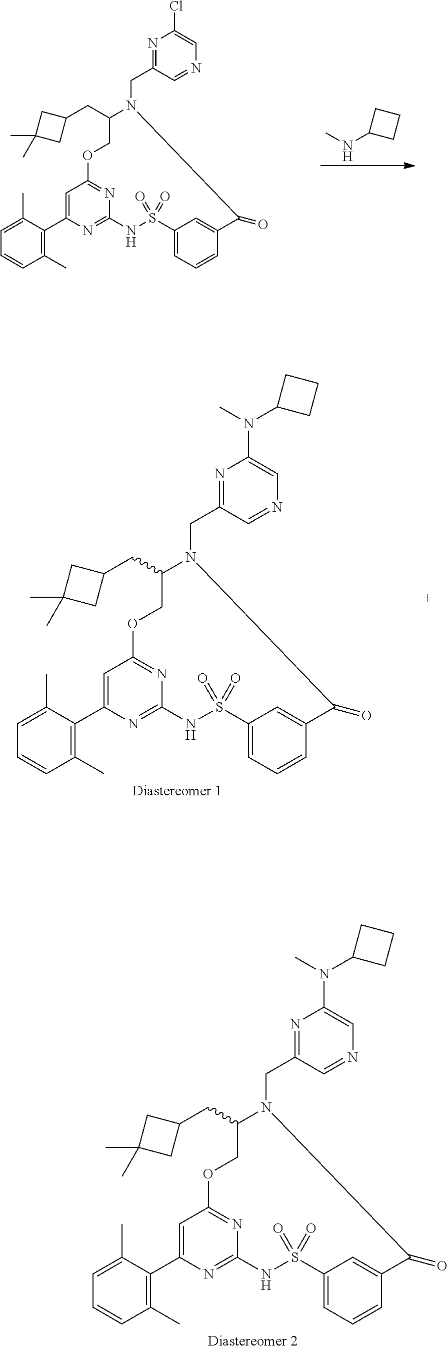

In a 4 mL vial, 12-[(6-chloropyrazin-2-yl)methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (60 mg, 0.09271 mmol) and N-methylcyclobutanamine (hydrochloride salt) (23 mg, 0.1891 mmol) were combined in DMSO (0.25 mL). Finely ground potassium carbonate (38 mg, 0.2750 mmol) was added. The reaction mixture was stirred at 120° C. overnight. After filtration, purification by reverse phase HPLC (30-99% acetonitrile/5 mM aqueous HCl over 15 minutes) gave 12-[[6-[cyclobutyl(methyl)amino]pyrazin-2-yl]methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (47.2 mg, 73%) as an off-white solid. ESI-MS m/z calc. 695.3254, found 696.5 (M+1)⁺; Retention time: 1.73 minutes. The diastereomers were separated by chiral SFC using a ChiralPak AS (4.6×250 mm, 5 μm) at 40° C. Mobile phase was 42% MeOH+20 mm NH₃ at a 70 mL/min flow. Concentration of the sample was 21.3 mg/mL (MeOH:DMSO; 76:24), injection volume 500 μL with an outlet pressure of 191 bar, detection wavelength of 210 nm. This provided 12-[[6-[cyclobutyl(methyl)amino]pyrazin-2-yl]methyl]-11-[(3,3-dimethylcyclobutyl)methyl]-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one (12.1 mg, 37%), ¹H NMR (400 MHz, Chloroform-d) δ 8.75 (s, 1H), 8.04 (d, J 7.9 Hz, 1H), 7.89 (d, J 11.5 Hz, 2H), 7.84 (d, J 7.7 Hz, 1H), 7.64 (t, J 7.8 Hz, 1H), 7.21 (t, J 7.6 Hz, 1H), 7.06 (d, J 7.7 Hz, 2H), 6.20 (s, 1H), 5.41-5.32 (m, 1H), 5.18 (d, J 15.6 Hz, 1H), 4.55 (p, J 8.6 Hz, 1H), 4.06-4.01 (m, 2H), 3.13 (s, 3H), 2.32-2.25 (m, 2H), 2.24-2.17 (m, 2H), 2.04 (s, 7H), 1.87-1.81 (m, 2H), 1.75-1.70 (m, 2H), 1.61-1.53 (m, 2H), 1.40-1.33 (m, 2H), 1.04 (s, 3H), 0.90 (s, 3H), 0.83-0.76 (m, 1H), ESI-MS m/z calc. 695.3254, found 696.6 (M+1)⁺; Retention time: 1.73 minutes as diastereomer 1, Peak 1 as a yellow solid, and provided 12-[[6-[cyclobutyl(methyl)amino]pyrazin-2-yl]methyl]-11-[(3,3-dimethylcyclobutyl)methyl]-6-(2,6-dimethylphenyl)-2,2-dioxo-9-oxa-2?⁶-thia-3,5,12,19-tetrazatricyclo[12.3.1.14,8]nonadeca-1(18),4(19),5,7,14,16-hexaen-13-one (11.3 mg, 35%)¹H NMR (400 MHz, Chloroform-d) δ 8.75 (s, 1H), 8.04 (d, J 7.9 Hz, 1H), 7.89 (d, J 12.3 Hz, 2H), 7.83 (d, J 7.7 Hz, 1H), 7.64 (t, J 7.7 Hz, 1H), 7.21 (t, J 7.6 Hz, 1H), 7.06 (d, J 7.7 Hz, 2H), 6.20 (s, 1H), 5.41-5.31 (m, 1H), 5.18 (d, J 15.6 Hz, 1H), 4.55 (p, J 8.5 Hz, 1H), 4.06-4.01 (m, 2H), 3.13 (s, 3H), 2.32-2.25 (m, 2H), 2.24-2.16 (m, 2H), 2.04 (s, 7H), 1.86-1.79 (m, 2H), 1.76-1.69 (m, 2H), 1.62-1.53 (m, 2H), 1.40-1.32 (m, 2H), 1.05 (s, 3H), 0.90 (s, 3H), 0.80 (t, J 10.9, 8.6 Hz, 1H). ESI-MS m/z calc. 695.3254, found 696.6 (M+1)⁺; Retention time: 1.73 minutes as diastereomer 2, Peak 2 as a yellow solid; LC method A.

### Example 175: Preparation of Compound 1341

### Step 1: 01-Benzyl 02-methyl (2R)-aziridine-1,2-dicarboxylate

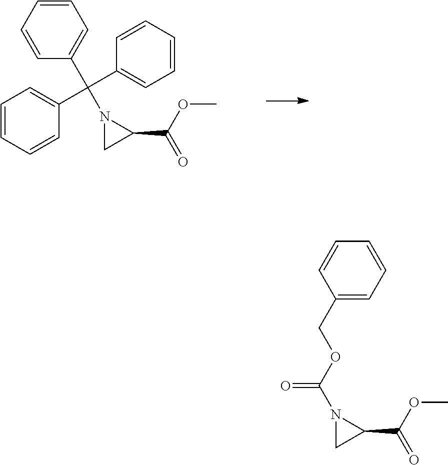 [SMILES: COC(=O)[C@H]1CN1C(c1ccccc1)(c1ccccc1)c1ccccc1 ; COC(=O)[C@H]1CN1C(=O)OCc1ccccc1]

TFA (35.520 g, 24 mL, 311.52 mmol) was added over a period of 10 minutes to a solution of methyl (2R)-1-tritylaziridine-2-carboxylate (6.6 g, 19.219 mmol) in a mixture of chloroform (24 mL) and methanol (24 mL) at 0° C. The reaction was stirred in an ice bath under nitrogen for 4 hours. The solvent was removed in vacuo at 0° C. The last traces of TFA were removed by azeotroping with diethyl ether (3×30 mL). The residue was partitioned between ether (30 mL) and water (30 mL). The ether layer was extracted with water (3×30 mL) and the combined aqueous extracts were made basic with sodium bicarbonate (10.5 g, 124.99 mmol). ethyl acetate (150 mL) was added to the aqueous layers and the mixture was cooled to 0° C. benzyl chloroformate (3.3600 g, 2.8 mL, 19.696 mmol) was added to the mixture, and the reaction was stirred at rt for 20 hours. The layers were separated, and the aqueous layer was extracted with ethyl acetate (3×50 mL). The combined organic layers were washed with brine (2×50 mL) and dried over anhydrous sodium sulfate. The solution was concentrated under vacuum to furnish 01-benzyl 02-methyl (2R)-aziridine-1,2-dicarboxylate (4.436 g, 98%) as a clear liquid. ¹H NMR (500 MHz, Chloroform-d) δ 7.47-7.27 (m, 5H), 5.15 (d, J 1.6 Hz, 2H), 3.71 (s, 3H), 3.11 (dd, J 5.5, 3.2 Hz, 1H), 2.60 (dd, J 3.2, 1.3 Hz, 1H), 2.48 (dd, J 5.5, 1.3 Hz, 1H).

### Step 2: Methyl (2R)-2-(benzyloxycarbonylamino)-3-(cyclopropoxy)propanoate

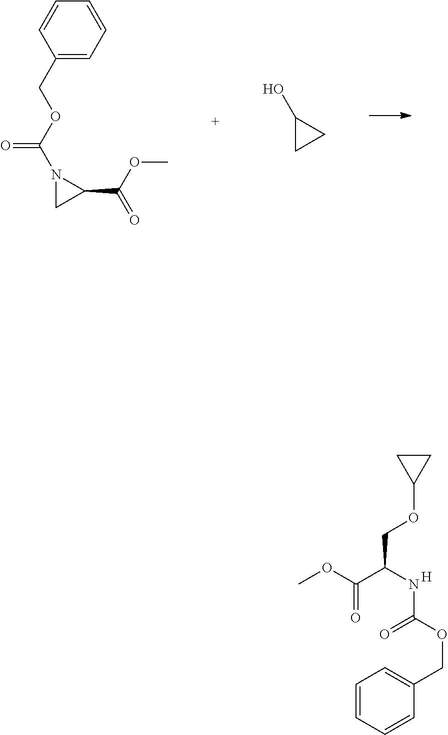

To a solution of 01-benzyl 02-methyl (2R)-aziridine-1,2-dicarboxylate (2 g, 8.5021 mmol) and cyclopropanol (1.8340 g, 2 mL, 31.578 mmol) in anhydrous DCM (20 mL) was added boron trifluoride diethyl etherate (115.00 mg, 0.1 mL, 0.8103 mmol) at 0° C. The reaction was stirred at rt overnight. The reaction mixture was diluted with water (50 mL) and DCM (50 mL). Two layers were separated, and the aqueous layer was extracted with DCM (2×50 mL). The combined organic layers were dried over anhydrous sodium sulfate and concentrated under vacuum. The residue was purified by silica gel chromatography using 0 to 20% acetone in hexane to furnish methyl (2R)-2-(benzyloxycarbonylamino)-3-(cyclopropoxy)propanoate (1.578 g, 63%) as a clear liquid. ESI-MS m/z calc. 293.1263, found 294.4 (M+1)⁺; LC method T.

### Step 3: Benzyl N-[(1S)-1-(cyclopropoxymethyl)-2-hydroxy-ethyl]carbamate

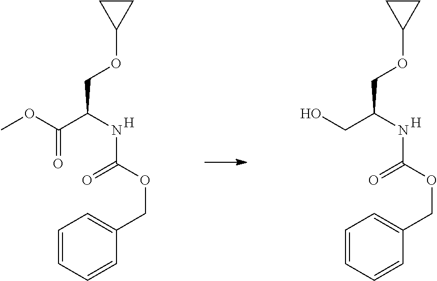

To a solution of methyl (2R)-2-(benzyloxycarbonylamino)-3-(cyclopropoxy)propanoate (1.578 g, 5.3799 mmol) in a solvent mixture of THF (15 mL) and methanol (5 mL) was added sodium borohydride (520 mg, 13.745 mmol). The reaction was stirred at rt for 3 hours. The residue was diluted with ethyl acetate (100 mL) and washed with brine (30 mL), dried over anhydrous sodium sulfate and concentrated under vacuum. The residue was purified by silica gel chromatography using 0 to 30% acetone in hexane to furnish benzyl N-[(1S)-1-(cyclopropoxymethyl)-2-hydroxy-ethyl]carbamate (1.138 g, 80%) as a white solidESI-MS m/z calc. 265.1314, found 266.2 (M+1)⁺; Retention time: 2.19 minutes; LC method T.

### Step 4: (2S)-2-Amino-3-(cyclopropoxy)propan-1-ol

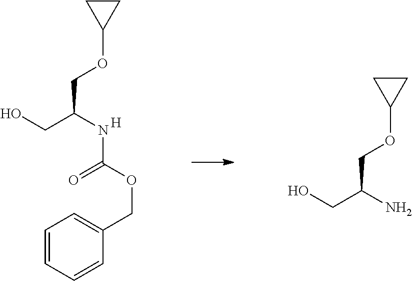

To a solution of benzyl N-[(1S)-1-(cyclopropoxymethyl)-2-hydroxy-ethyl]carbamate (1.138 g, 4.2894 mmol) in ethyl acetate (20 mL) was added 10% Pd/C (458 mg, 10% w/w, 0.4304 mmol). The reaction was hydrogenated under 1 atm of hydrogen gas for 3 hours. The catalyst was removed by filtration through a pad of Celite. The filtrate was concentrated under vacuum to furnish (2S)-2-amino-3-(cyclopropoxy)propan-1-ol (533 mg, 95%) as a clear gel. ¹H NMR (400 MHz, Acetone-d₆) δ 3.61 (dd, J 10.8, 4.5 Hz, 1H), 3.54 (dd, J 9.5, 4.8 Hz, 1H), 3.51-3.43 (m, 2H), 3.30 (tt, J 6.0, 3.0 Hz, 1H), 3.06 (tt, J 6.2, 4.6 Hz, 1H), 1.83 (s, 3H), 0.62-0.53 (m, 2H), 0.53-0.44 (m, 2H).

### Step 5: 3-[[4-[(2R)-2-Amino-3-(cyclopropoxy)propoxy]-6-(2,6-dimethylphenyl)pyrimidin-2-yl]sulfamoyl]benzoic acid

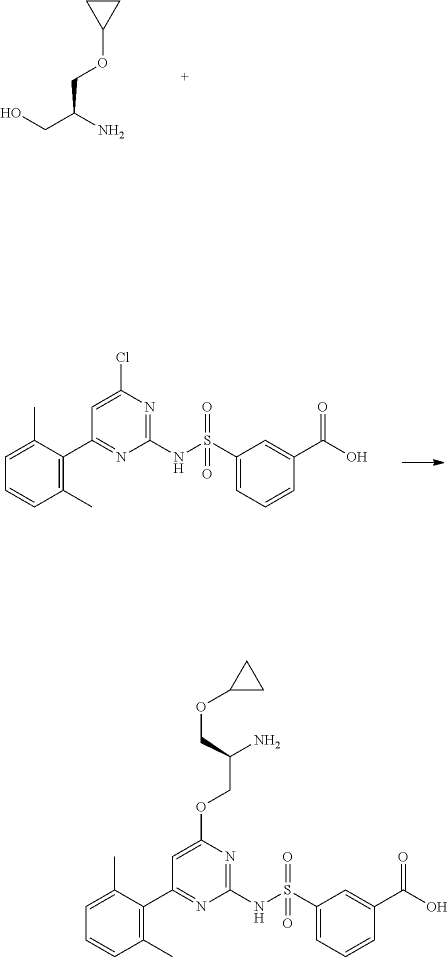 [SMILES: N[C@@H](CO)COC1CC1 ; Cc1cccc(C)c1-c1cc(Cl)nc(NS(=O)(=O)c2cccc(C(=O)O)c2)n1 ; Cc1cccc(C)c1-c1cc(OC[C@H](N)COC2CC2)nc(NS(=O)(=O)c2cccc(C(=O)O)c2)n1]

To a solution of 3-[[4-chloro-6-(2,6-dimethylphenyl)pyrimidin-2-yl]sulfamoyl]benzoic acid (1.615 g, 3.8649 mmol) and (2S)-2-amino-3-(cyclopropoxy)propan-1-ol (533 mg, 3.8602 mmol) in anhydrous THF (12 mL) was added tBuONa (2.56 g, 26.638 mmol) at rt. The reaction was stirred at rt for 1 hour. The reaction was quenched with 1 N HCl (35 mL). The product was extracted with ethyl acetate (3×35 mL). The combined organic layers were washed with brine (30 mL), dried over anhydrous sodium sulfate and concentrated under vacuum. The residue was triturated with 1:1 ethyl acetate and hexane (20 mL) to furnish 3-[[4-[(2R)-2-amino-3-(cyclopropoxy)propoxy]-6-(2,6-dimethylphenyl)pyrimidin-2-yl]sulfamoyl]benzoic acid (hydrochloride salt) (1.654 g, 70%) as an off white solid. ¹H NMR (400 MHz, DMSO-d₆) δ 13.38 (s, 1H), 8.43 (t, J=1.8 Hz, 1H), 8.26 (s, 3H), 8.19-8.08 (m, 2H), 7.70 (t, J 7.8 Hz, 1H), 7.31-7.19 (m, 1H), 7.13 (d, J 7.7 Hz, 2H), 6.32 (s, 1H), 4.41-4.27 (m, 2H), 3.78-3.70 (m, 2H), 3.70-3.62 (m, 1H), 3.45-3.39 (m, 1H), 1.99 (s, 6H), 0.61-0.54 (m, 2H), 0.53-0.43 (m, 2H). ESI-MS m/z calc. 512.173, found 513.4 (M+1)⁺; Retention time: 1.54 minutes; LC method W.

### Step 6: 3-[[4-[(2R)-2-[(6-Chloropyrazin-2-yl)methylamino]-3-(cyclopropoxy)propoxy]-6-(2,6-dimethylphenyl)pyrimidin-2-yl]sulfamoyl]benzoic acid

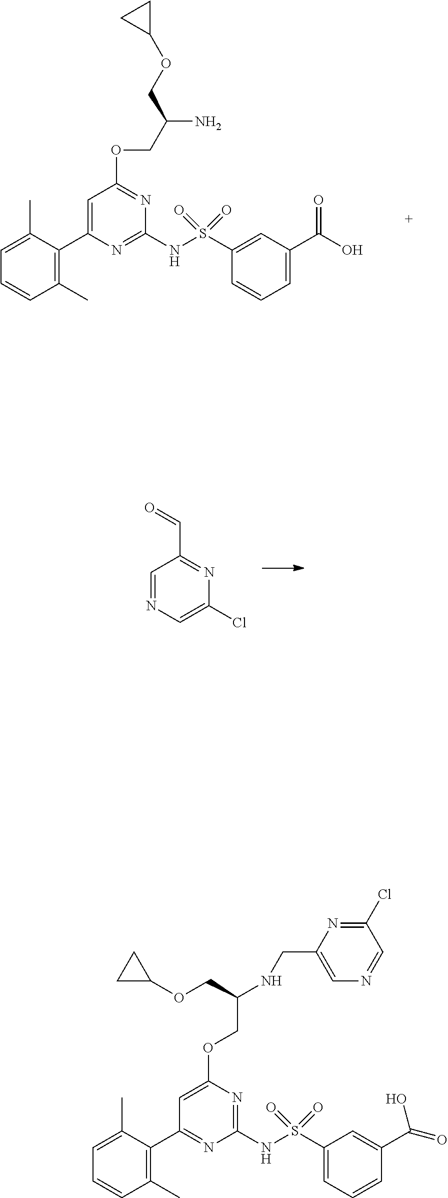 [SMILES: Cc1cccc(C)c1-c1cc(OC[C@H](N)COC2CC2)nc(NS(=O)(=O)c2cccc(C(=O)O)c2)n1 ; O=Cc1cncc(Cl)n1 ; Cc1cccc(C)c1-c1cc(OC[C@@H](COC2CC2)NCc2cncc(Cl)n2)nc(NS(=O)(=O)c2cccc(C(=O)O)c2)n1]

A 20 mL vial was charged with 3-[[4-[(2R)-2-amino-3-(cyclopropoxy)propoxy]-6-(2,6-dimethylphenyl)pyrimidin-2-yl]sulfamoyl]benzoic acid (hydrochloride salt) (600 mg, 0.9835 mmol), 6-chloropyrazine-2-carbaldehyde (148 mg, 1.038 mmol), anhydrous DCM (4.5 mL) and acetic acid (85 μL, 1.495 mmol). The mixture was cooled down in an ice bath. DIEA (375 μL, 2.153 mmol) was added, followed by sodium triacetoxyborohydride (1.05 g, 4.954 mmol) and the reaction was vigorously stirred at 0° C. After 90 minutes additional DCM (2 mL) was added to improve stirring. At 5 h reaction time additional sodium triacetoxyborohydride (200 mg, 0.9437 mmol) was added. After 5.5 hours total reaction time, the reaction mixture was quenched into 2M HCl and extracted 4× with ethyl acetate. The combined organics were washed with brine, dried over sodium sulfate, and concentrated. The resulting crude was dissolved in 1:2 DMSO/methanol, filtered, and purified by preparative HPLC (10-99% ACN in water, HCl modifier, shallow initial gradient to 60%, 20 minute run) to give 3-[[4-[(2R)-2-[(6-chloropyrazin-2-yl)methylamino]-3-(cyclopropoxy)propoxy]-6-(2,6-dimethylphenyl)pyrimidin-2-yl]sulfamoyl]benzoic acid (hydrochloride salt) (435 mg, 65%) as a white solid. ESI-MS m/z calc. 638.17145, found 639.6 (M+1)⁺; Retention time: 0.48 minutes; LC method D.

### Step 7: (11R)-12-[(6-Chloropyrazin-2-yl)methyl]-11-(cyclopropoxymethyl)-6-(2,6-dimethylphenyl)-2,2-dioxo-9-oxa-2λ⁶-thia-3,5,12,19-tetrazatricyclo[12.3.1.14,8]nonadeca-1(18),4(19),5,7,14,16-hexaen-13-one

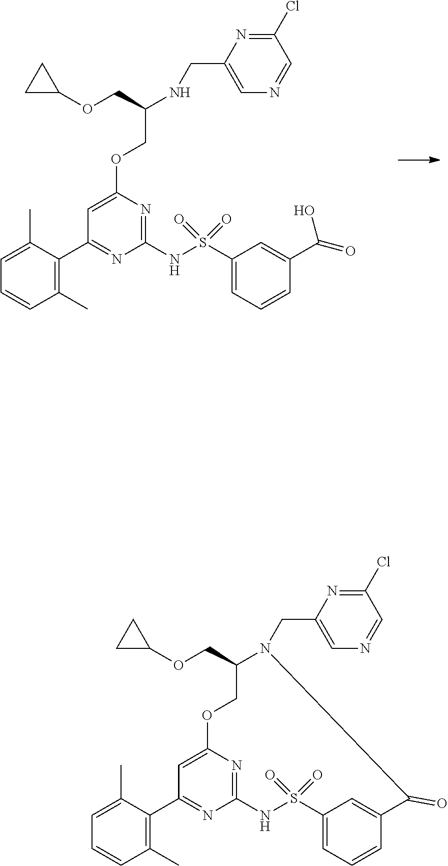 [SMILES: Cc1cccc(C)c1-c1cc(OC[C@@H](COC2CC2)NCc2cncc(Cl)n2)nc(NS(=O)(=O)c2cccc(C(=O)O)c2)n1 ; Cc1cccc(C)c1-c1cc2nc(n1)NS(=O)(=O)c1cccc(c1)C(=O)N(Cc1cncc(Cl)n1)[C@H](COC1CC1)CO2]

3-[[4-[(2R)-2-[(6-Chloropyrazin-2-yl)methylamino]-3-(cyclopropoxy)propoxy]-6-(2,6-dimethylphenyl)pyrimidin-2-yl]sulfamoyl]benzoic acid (hydrochloride salt) (435 mg, 0.6439 mmol) was combined with CDMT (145 mg, 0.8259 mmol) in DMF (50 mL) and cooled to 0° C. N-methylmorpholine (430 μL, 3.911 mmol) was added by syringe and the reaction was stirred at 0° C. for 60 minutes. The ice bath was then removed and stirring was continued for an additional 20 hours at room temperature. The reaction mixture was then concentrated by rotary evaporation (bath temp 50° C.). The resulting residue was partitioned between 50 mL 1M HCl and 50 mL ethyl acetate. The layers were separated and the aqueous was extracted with an additional 3×50 mL ethyl acetate. The combined organics were washed 1×50 mL with brine, dried over sodium sulfate, filtered, and concentrated. The crude product was purified by flash chromatography on silica gel eluting with a 0-10% methanol in DCM gradient to give as a white solid (11R)-12-[(6-chloropyrazin-2-yl)methyl]-11-(cyclopropoxymethyl)-6-(2,6-dimethylphenyl)-2,2-dioxo-9-oxa-2?⁶-thia-3,5,12,19-tetrazatricyclo[12.3.1.14,8]nonadeca-1(18),4(19),5,7,14,16-hexaen-13-one (412 mg, 97%), ESI-MS m/z calc. 620.1609, found 621.5 (M+1)⁺; Retention time: 0.68 minutes; LC method D.

### Step 8: (11R)-11-(Cyclopropoxymethyl)-6-(2,6-dimethylphenyl)-12-[[6-[(2S)-2-isopropylpyrrolidin-1-yl]pyrazin-2-yl]methyl]-2,2-dioxo-9-oxa-2⁶-thia-3,5,12,19-tetrazatricyclo[12.3.1.14,8]nonadeca-1(18),4(19),5,7,14,16-hexaen-13-one (Compound 1341)

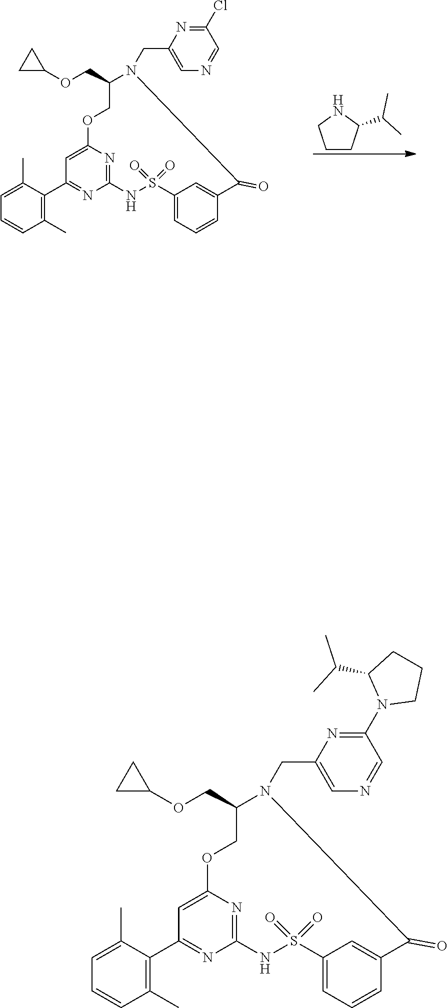

(11R)-12-[(6-Chloropyrazin-2-yl)methyl]-11-(cyclopropoxymethyl)-6-(2,6-dimethylphenyl)-2,2-dioxo-9-oxa-2?⁶-thia-3,5,12,19-tetrazatricyclo[12.3.1.14,8]nonadeca-1(18),4(19),5,7,14,16-hexaen-13-one (18 mg, 0.02724 mmol) was combined with (2S)-2-isopropylpyrrolidine (hydrochloride salt) (24 mg, 0.1604 mmol) and potassium carbonate (50 mg, 0.3618 mmol) (325 mesh) in DMSO (0.3 mL) and dioxane (0.1 mL) in a screwcap vial and heated at 120° C. for 48 hours. The reaction mixture was then cooled to room temperature, diluted with methanol, filtered, and purified by preparative HPLC (1-99% ACN in water, HCl modifier, 15-minute run). Fractions containing product were diluted slightly with brine and extracted with ethyl acetate. The organics were dried over sodium sulfate, and concentrated to give (11R)-11-(cyclopropoxymethyl)-6-(2,6-dimethylphenyl)-12-[[6-[(2S)-2-isopropylpyrrolidin-1-yl]pyrazin-2-yl]methyl]-2,2-dioxo-9-oxa-2?⁶-thia-3,5,12,19-tetrazatricyclo[12.3.1.14,8]nonadeca-1(18),4(19),5,7,14,16-hexaen-13-one (7.8 mg, 41%). ESI-MS m/z calc. 697.3046, found 698.7 (M+1)⁺; Retention time: 1.81 minutes; LC method A. ¹H NMR (400 MHz, Chloroform-d) δ 8.71 (s, 1H), 7.90-7.77 (m, 4H), 7.59 (t, J 7.7 Hz, 1H), 7.22 (d, J 7.5 Hz, 1H), 7.09 (d, J 7.6 Hz, 2H), 6.26 (s, 1H), 5.40 (d, J 6.3 Hz, 1H), 5.10 (d, J 15.4 Hz, 1H), 4.35 (d, J 6.0 Hz, 2H), 4.26 (d, J 15.2 Hz, 1H), 4.06 (q, J 5.3 Hz, 1H), 3.73-3.64 (m, 2H), 3.62-3.54 (m, 2H), 3.23-3.16 (m, 1H), 2.40-2.29 (m, 1H), 2.08 (s, 6H), 2.04-1.92 (m, 4H), 0.91 (d, J 6.9 Hz, 3H), 0.81 (d, J 6.8 Hz, 3H), 0.53-0.41 (m, 4H). (Sulfonamide NH not visible).

### Example 176: Preparation of Compound 1342

### Step 1: (11R)-6-(2,6-Dimethylphenyl)-11-(2,2-dimethylpropyl)-12-[[6-(methylamino)-2-pyridyl]methyl]-2,2-dioxo-9-oxa-2λ⁶-thia-3,5,12,19-tetrazatricyclo[12.3.1.14,8]nonadeca-1(18),4(19),5,7,14,16-hexaen-13-one

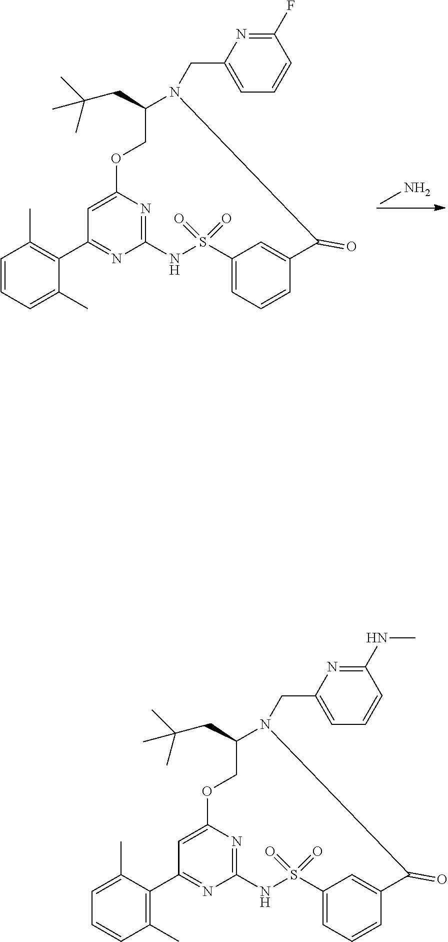

(11R)-6-(2,6-Dimethylphenyl)-11-(2,2-dimethylpropyl)-12-[(6-fluoropyridin-2-yl)methyl]-9-oxa-2λ⁶-thia-3, 5,12,19-tetraazatricyclo[12.3.1.14,8]nonadeca-1(17),4,6,8(19),14(18),15-hexaene-2,2,13-trione (250 mg, 0.4141 mmol) was combined with methylamine (hydrochloride salt) (112 mg, 1.659 mmol) and freshly ground potassium carbonate (570 mg, 4.124 mmol) in a screwcap vial with an unpierced septum. DMSO (500 μL) and dioxane (0.1 mL) were added and the reaction was heated to 120° C. for 5 hours. The reaction was then cooled to room temperature, diluted with methanol, filtered, and purified by reverse phase preparative HPLC (1-99% ACN in water, HCl modifier) to give (11R)-6-(2,6-dimethylphenyl)-11-(2,2-dimethylpropyl)-12-[[6-(methylamino)-2-pyridyl]methyl]-2,2-dioxo-9-oxa-2?⁶-thia-3,5,12,19-tetrazatricyclo[12.3.1.14,8]nonadeca-1(18),4(19),5,7,14,16-hexaen-13-one (180.2 mg, 71%). ESI-MS m/z calc. 614.2675, found 0.52 (M+1)⁺; Retention time: 615.9 minutes; LC method D.

### Step 2: Isopropyl N-[6-[[(11R)-6-(2,6-dimethylphenyl)-11-(2,2-dimethylpropyl)-2,2,13-trioxo-9-oxa-2λ⁶-thia-3,5,12,19-tetrazatricyclo[12.3.1.14,8]nonadeca-1(18),4(19),5,7,14,16-hexaen-12-yl]methyl]-2-pyridyl]-N-methyl-carbamate (Compound 1342)

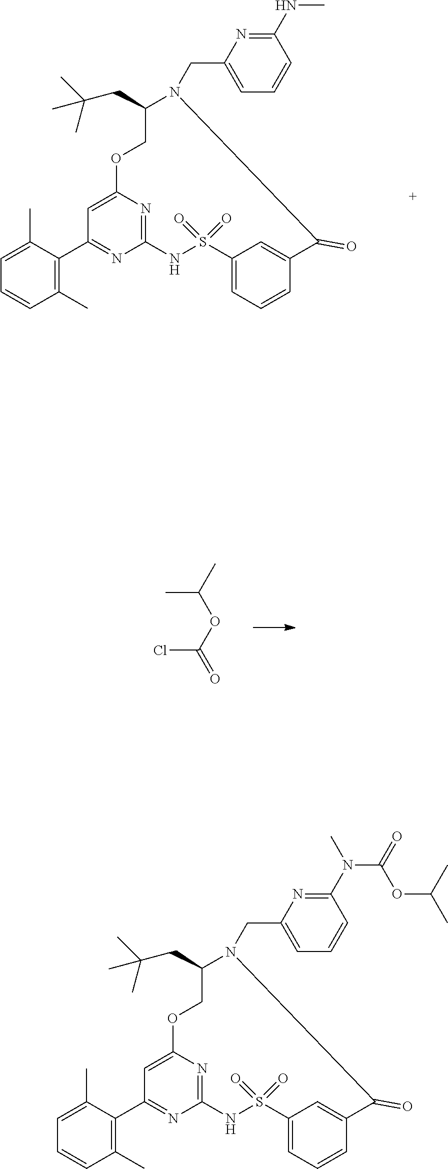

(11R)-6-(2,6-Dimethylphenyl)-11-(2,2-dimethylpropyl)-12-[[6-(methylamino)-2-pyridyl]methyl]-2,2-dioxo-9-oxa-2?⁶-thia-3,5,12,19-tetrazatricyclo[12.3.1.14,8]nonadeca-1(18),4(19),5,7,14,16-hexaen-13-one (25 mg, 0.04067 mmol) was combined with isopropyl chloroformate (70 μL of 2 M, 0.1400 mmol) in DCM (0.5 mL). DIEA (50 μL, 0.2871 mmol) was added and the reaction was stirred for 2 hours at room temperature. DMAP (0.5 mg, 0.004093 mmol) was added, and the reaction was stirred for an additional 22 hours at room temperature. The reaction mixture was then quenched with 2 drops 1M HCl, diluted with methanol, filtered, and purified by preparative HPLC (1-99% ACN in water, HCl modifier) to give isopropyl N-[6-[[(11R)-6-(2,6-dimethylphenyl)-11-(2,2-dimethylpropyl)-2,2,13-trioxo-9-oxa-2?⁶-thia-3,5,12,19-tetrazatricyclo[12.3.1.14,8]nonadeca-1(18),4(19),5,7,14,16-hexaen-12-yl]methyl]-2-pyridyl]-N-methyl-carbamate (1.8 mg, 6%). ESI-MS m/z calc. 700.3043, found 701.8 (M+1)⁺; Retention time: 2.01 minutes; LC method A.

C. Characterization of Compounds 1295-1972

The compounds in Tables 14-16 below were prepared by procedures analogous to those disclosed in the specification, and the analytical data were consistent with the reported structure.

TABLE 14
LCMS Data

| | | LCMS | | | |
|---|---|---|---|---|---|
| Compound | | Rt | Calc. | | LCMS |
| Number | Structure | (min) | mass | M + 1 | Method |
| 1343 | 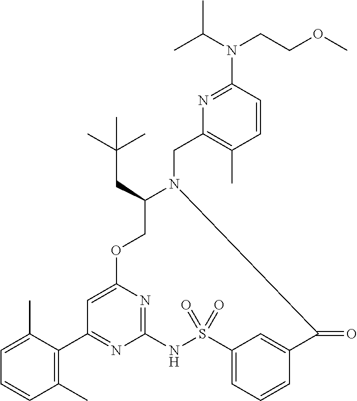 [SMILES: COCCN(c1ccc(C)c(CN2C(=O)c3cccc(c3)S(=O)(=O)Nc3nc(cc(-c4c(C)cccc4C)n3)OC[C@H]2CC(C)(C)C)n1)C(C)C] | 1.64 | 714.356 | 715.6 | A |
| 1344 | 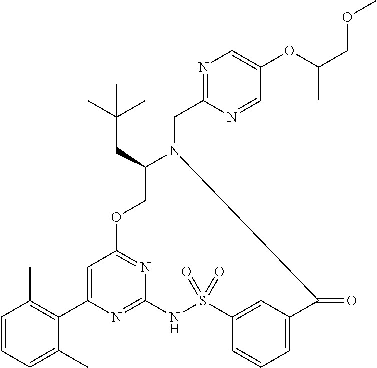 [SMILES: COCC(C)Oc1cnc(CN2C(=O)c3cccc(c3)S(=O)(=O)Nc3nc(cc(-c4c(C)cccc4C)n3)OC[C@H]2CC(C)(C)C)nc1] | 0.7 | 674.289 | 675.5 | D |
| 1345 | 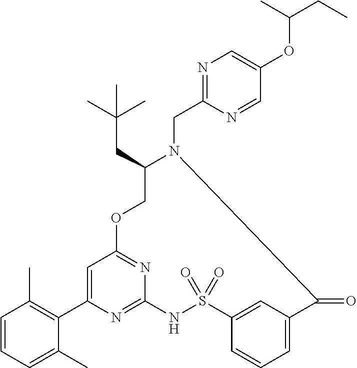 [SMILES: CCC(C)Oc1cnc(CN2C(=O)c3cccc(c3)S(=O)(=O)Nc3nc(cc(-c4c(C)cccc4C)n3)OC[C@H]2CC(C)(C)C)nc1] | 0.77 | 658.294 | 659.7 | D |
| 1346 | 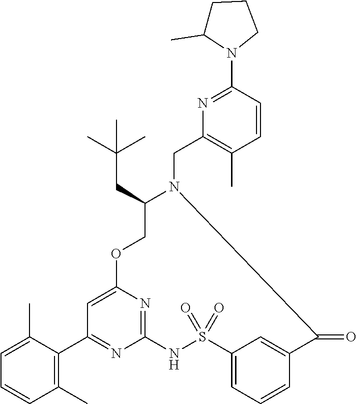 [SMILES: Cc1ccc(N2CCCC2C)nc1CN1C(=O)c2cccc(c2)S(=O)(=O)Nc2nc(cc(-c3c(C)cccc3C)n2)OC[C@H]1CC(C)(C)C] | 1.52 | 682.33 | 683.5 | A |
| 1347 | 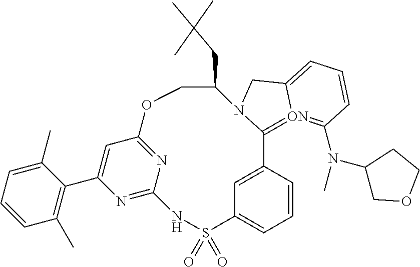 | 1.45 | 684.309 | 685.6 | A |
| 1348 | 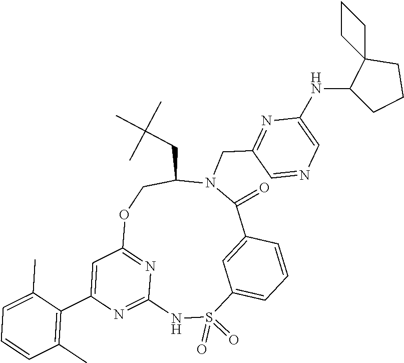 [SMILES: Cc1cccc(C)c1-c1cc2nc(n1)NS(=O)(=O)c1cccc(c1)C(=O)N(Cc1cncc(NC3CCCC34CCC4)n1)[C@H](CC(C)(C)C)CO2] | 1.96 | 709.341 | 710.6 | A |
| 1349 | 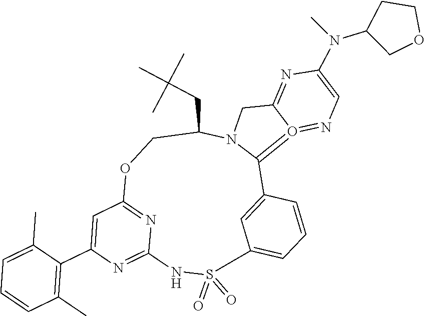 | 1.66 | 685.305 | 686.6 | A |
| 1350 | 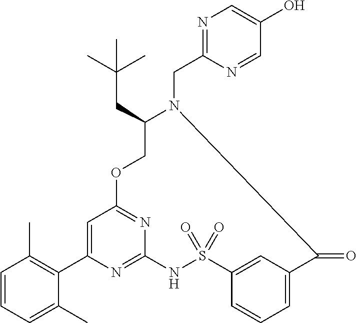 [SMILES: Cc1cccc(C)c1-c1cc2nc(n1)NS(=O)(=O)c1cccc(c1)C(=O)N(Cc1ncc(O)cn1)[C@H](CC(C)(C)C)CO2] | 0.63 | 602.231 | 603.4 | D |
| 1352 | 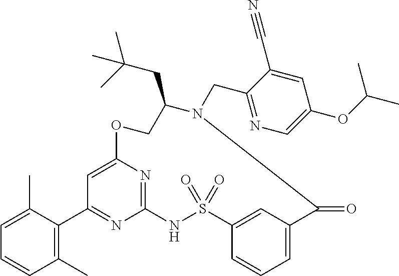 [SMILES: Cc1cccc(C)c1-c1cc2nc(n1)NS(=O)(=O)c1cccc(c1)C(=O)N(Cc1ncc(OC(C)C)cc1C#N)[C@H](CC(C)(C)C)CO2] | 2.02 | 668.278 | 669.5 | A |
| 1352 | 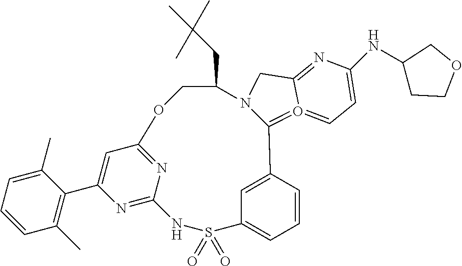 | 1.34 | 670.294 | 671.6 | A |
| 1353 | 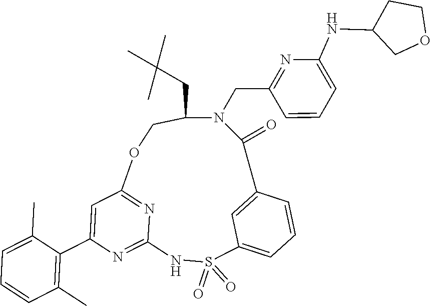 [SMILES: Cc1cccc(C)c1-c1cc2nc(n1)NS(=O)(=O)c1cccc(c1)C(=O)N(Cc1cccc(NC3CCOC3)n1)[C@H](CC(C)(C)C)CO2] | 1.589 | 671.289 | 672.57 | A |
| 1354 | 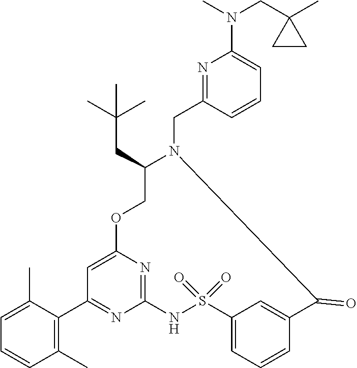 [SMILES: Cc1cccc(C)c1-c1cc2nc(n1)NS(=O)(=O)c1cccc(c1)C(=O)N(Cc1cccc(N(C)CC3(C)CC3)n1)[C@H](CC(C)(C)C)CO2] | 1.52 | 682.33 | 683.5 | A |
| 1355 | 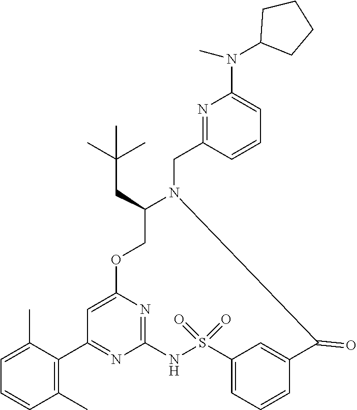 [SMILES: Cc1cccc(C)c1-c1cc2nc(n1)NS(=O)(=O)c1cccc(c1)C(=O)N(Cc1cccc(N(C)C3CCCC3)n1)[C@H](CC(C)(C)C)CO2] | 1.57 | 682.33 | 683.5 | A |
| 1356 | 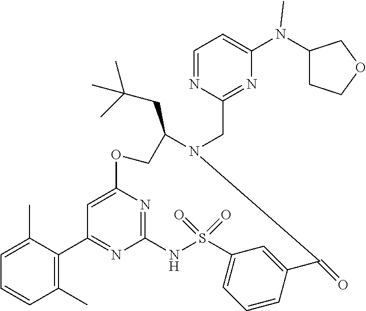 [SMILES: Cc1cccc(C)c1-c1cc2nc(n1)NS(=O)(=O)c1cccc(c1)C(=O)N(Cc1nccc(N(C)C3CCOC3)n1)[C@H](CC(C)(C)C)CO2] | 2.65 | 685.305 | 686.3 | Y |
| 1357 | 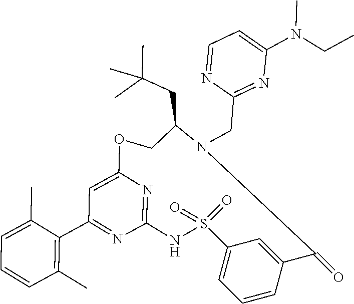 [SMILES: CCN(C)c1ccnc(CN2C(=O)c3cccc(c3)S(=O)(=O)Nc3nc(cc(-c4c(C)cccc4C)n3)OC[C@H]2CC(C)(C)C)n1] | 2.63 | 643.294 | 644.3 | Y |
| 1358 | 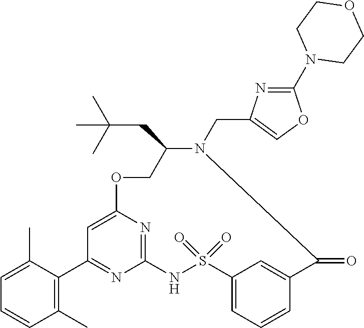 [SMILES: Cc1cccc(C)c1-c1cc2nc(n1)NS(=O)(=O)c1cccc(c1)C(=O)N(Cc1coc(N3CCOCC3)n1)[C@H](CC(C)(C)C)CO2] | 4.13 | 660.273 | 661.3 | Y |
| 1359 | 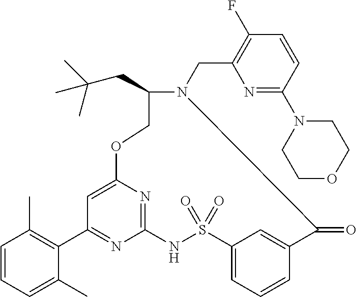 [SMILES: Cc1cccc(C)c1-c1cc2nc(n1)NS(=O)(=O)c1cccc(c1)C(=O)N(Cc1nc(N3CCOCC3)ccc1F)[C@H](CC(C)(C)C)CO2] | 1.94 | 688.284 | 689.5 | A |
| 1360 | 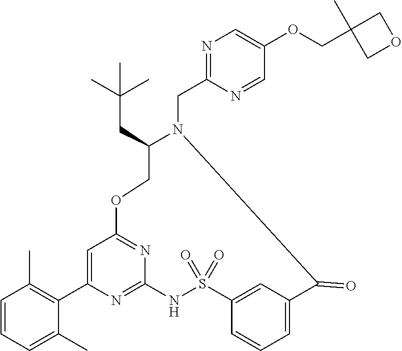 [SMILES: Cc1cccc(C)c1-c1cc2nc(n1)NS(=O)(=O)c1cccc(c1)C(=O)N(Cc1ncc(OCC3(C)COC3)cn1)[C@H](CC(C)(C)C)CO2] | 0.46 | 686.289 | 687.5 | D(30- 99% gradient) |
| 1361 | 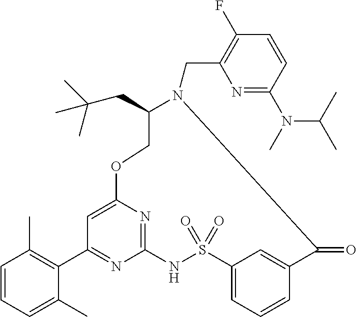 [SMILES: Cc1cccc(C)c1-c1cc2nc(n1)NS(=O)(=O)c1cccc(c1)C(=O)N(Cc1nc(N(C)C(C)C)ccc1F)[C@H](CC(C)(C)C)CO2] | 1.94 | 674.305 | 675.5 | A |
| 1362 | 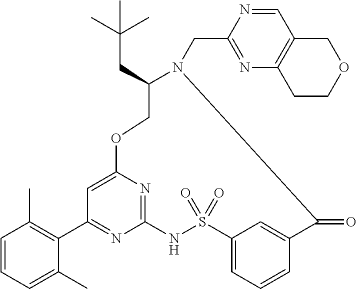 [SMILES: Cc1cccc(C)c1-c1cc2nc(n1)NS(=O)(=O)c1cccc(c1)C(=O)N(Cc1ncc3c(n1)CCOC3)[C@H](CC(C)(C)C)CO2] | 1.64 | 642.262 | 643.2 | A |
| 1363 | 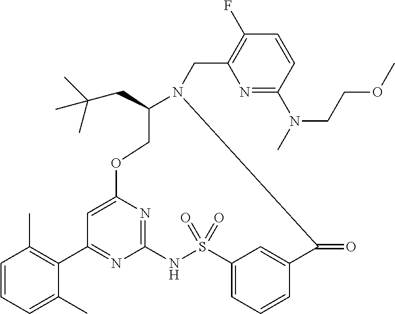 [SMILES: COCCN(C)c1ccc(F)c(CN2C(=O)c3cccc(c3)S(=O)(=O)Nc3nc(cc(-c4c(C)cccc4C)n3)OC[C@H]2CC(C)(C)C)n1] | 1.93 | 690.3 | 691.5 | A |
| 1364 | 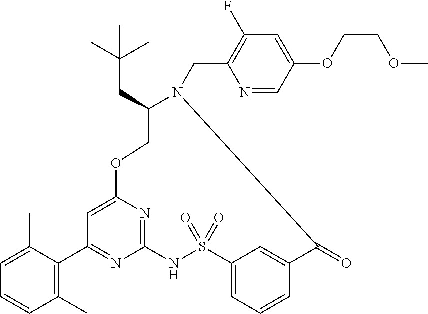 [SMILES: COCCOc1cnc(CN2C(=O)c3cccc(c3)S(=O)(=O)Nc3nc(cc(-c4c(C)cccc4C)n3)OC[C@H]2CC(C)(C)C)c(F)c1] | 1.66 | 677.268 | 678.5 | A |
| 1365 | 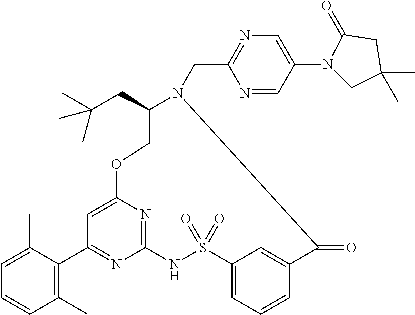 [SMILES: Cc1cccc(C)c1-c1cc2nc(n1)NS(=O)(=O)c1cccc(c1)C(=O)N(Cc1ncc(N3CC(C)(C)CC3=O)cn1)[C@H](CC(C)(C)C)CO2] | 1.79 | 697.305 | 698.6 | A |
| 1366 | 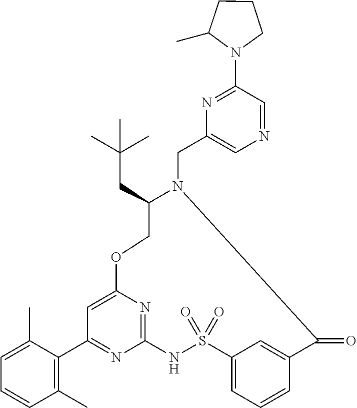 [SMILES: Cc1cccc(C)c1-c1cc2nc(n1)NS(=O)(=O)c1cccc(c1)C(=O)N(Cc1cncc(N3CCCC3C)n1)[C@H](CC(C)(C)C)CO2] | 1.54 | 669.31 | 670.5 | A |
| 1367 | 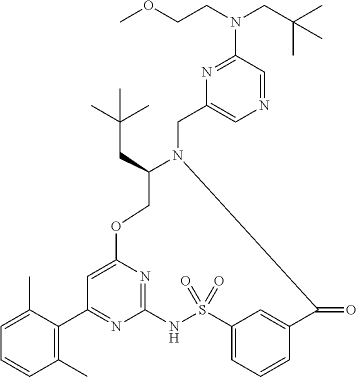 [SMILES: COCCN(CC(C)(C)C)c1cncc(CN2C(=O)c3cccc(c3)S(=O)(=O)Nc3nc(cc(-c4c(C)cccc4C)n3)OC[C@H]2CC(C)(C)C)n1] | 1.72 | 729.367 | 730.6 | A |
| 1368 | 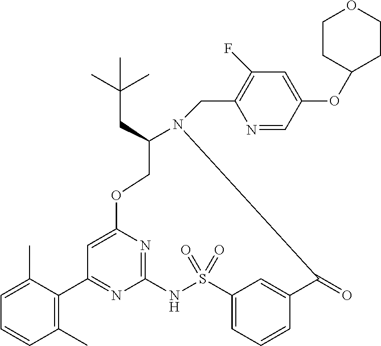 [SMILES: Cc1cccc(C)c1-c1cc2nc(n1)NS(=O)(=O)c1cccc(c1)C(=O)N(Cc1ncc(OC3CCOCC3)cc1F)[C@H](CC(C)(C)C)CO2] | 1.7 | 703.284 | 704.5 | A |
| 1369 | 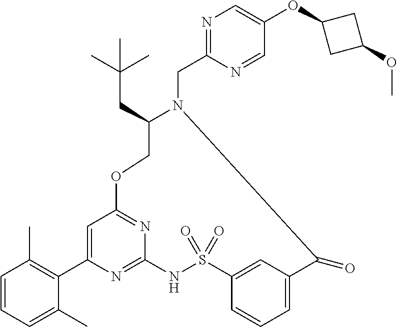 [SMILES: CO[C@H]1C[C@@H](Oc2cnc(CN3C(=O)c4cccc(c4)S(=O)(=O)Nc4nc(cc(-c5c(C)cccc5C)n4)OC[C@H]3CC(C)(C)C)nc2)C1] | 0.71 | 686.289 | 687.6 | D |
| 1370 | 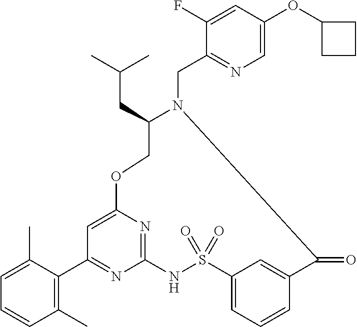 [SMILES: Cc1cccc(C)c1-c1cc2nc(n1)NS(=O)(=O)c1cccc(c1)C(=O)N(Cc1ncc(OC3CCC3)cc1F)[C@H](CC(C)C)CO2] | 2.07 | 659.258 | 660.5 | A |
| 1299 | 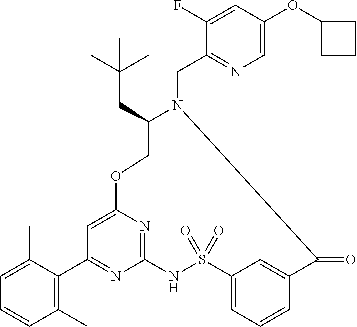 [SMILES: Cc1cccc(C)c1-c1cc2nc(n1)NS(=O)(=O)c1cccc(c1)C(=O)N(Cc1ncc(OC3CCC3)cc1F)[C@H](CC(C)(C)C)CO2] | 2.11 | 673.273 | 674.5 | A |
| 1371 | 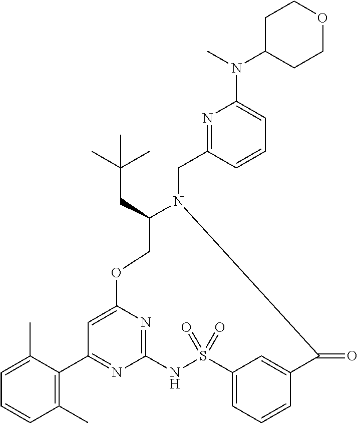 [SMILES: Cc1cccc(C)c1-c1cc2nc(n1)NS(=O)(=O)c1cccc(c1)C(=O)N(Cc1cccc(N(C)C3CCOCC3)n1)[C@H](CC(C)(C)C)CO2] | 1.4 | 698.325 | 699.4 | A |
| 1372 | 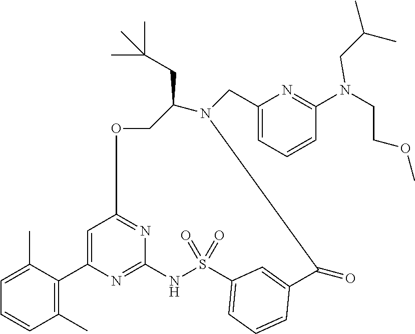 [SMILES: COCCN(CC(C)C)c1cccc(CN2C(=O)c3cccc(c3)S(=O)(=O)Nc3nc(cc(-c4c(C)cccc4C)n3)OC[C@H]2CC(C)(C)C)n1] | 98.4 | 714.356 | 715.6 | W |
| 1373 | 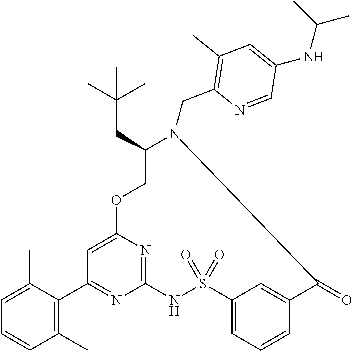 [SMILES: Cc1cc(NC(C)C)cnc1CN1C(=O)c2cccc(c2)S(=O)(=O)Nc2nc(cc(-c3c(C)cccc3C)n2)OC[C@H]1CC(C)(C)C] | 1.46 | 656.314 | 657.7 | A |
| 1374 | 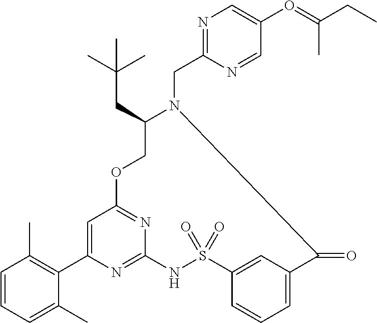 | 0.56 | 658.294 | 659.5 | D(30- 99% gradient) |
| 1375 | 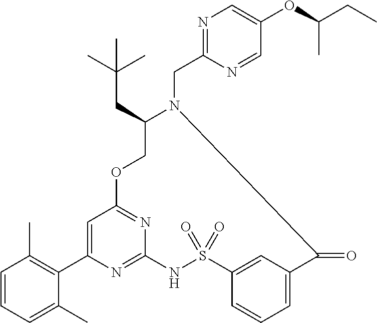 [SMILES: CC[C@@H](C)Oc1cnc(CN2C(=O)c3cccc(c3)S(=O)(=O)Nc3nc(cc(-c4c(C)cccc4C)n3)OC[C@H]2CC(C)(C)C)nc1] | 0.56 | 658.294 | 659.5 | D(30- 99% gradient) |
| 1376 | 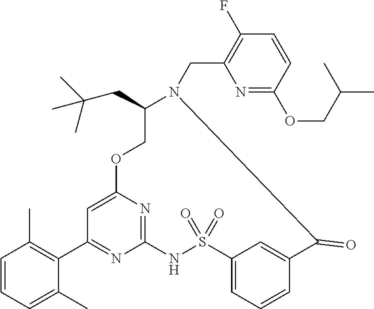 [SMILES: Cc1cccc(C)c1-c1cc2nc(n1)NS(=O)(=O)c1cccc(c1)C(=O)N(Cc1nc(OCC(C)C)ccc1F)[C@H](CC(C)(C)C)CO2] | 2.18 | 675.289 | 676.5 | A |
| 1300 | 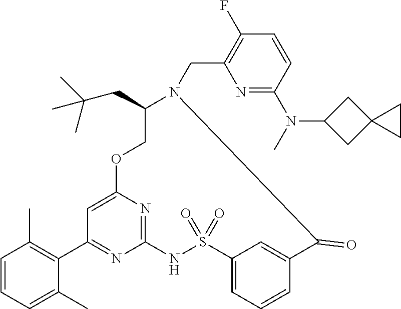 [SMILES: Cc1cccc(C)c1-c1cc2nc(n1)NS(=O)(=O)c1cccc(c1)C(=O)N(Cc1nc(N(C)C3CC4(CC4)C3)ccc1F)[C@H](CC(C)(C)C)CO2] | 2.14 | 712.321 | 713.6 | A |
| 1301 | 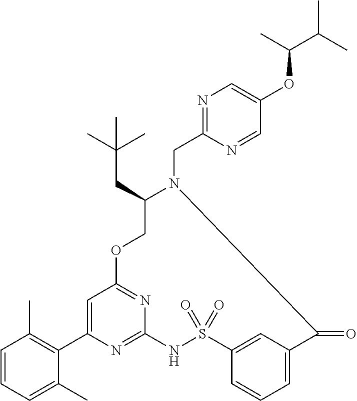 [SMILES: Cc1cccc(C)c1-c1cc2nc(n1)NS(=O)(=O)c1cccc(c1)C(=O)N(Cc1ncc(O[C@@H](C)C(C)C)cn1)[C@H](CC(C)(C)C)CO2] | 2.05 | 672.309 | 673.2 | A |
| 1377 | 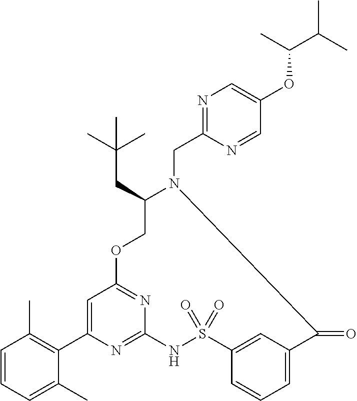 [SMILES: Cc1cccc(C)c1-c1cc2nc(n1)NS(=O)(=O)c1cccc(c1)C(=O)N(Cc1ncc(O[C@H](C)C(C)C)cn1)[C@H](CC(C)(C)C)CO2] | 2.05 | 672.309 | 673.2 | A |
| 1302 | 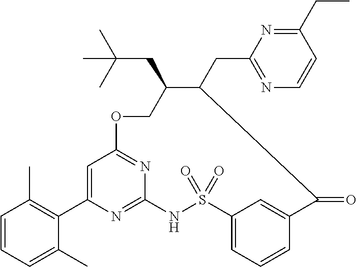 [SMILES: CCc1ccnc(CC2C(=O)c3cccc(c3)S(=O)(=O)Nc3nc(cc(-c4c(C)cccc4C)n3)OC[C@H]2CC(C)(C)C)n1] | 4.23 | 614.268 | 615.3 | Y |
| 1378 | 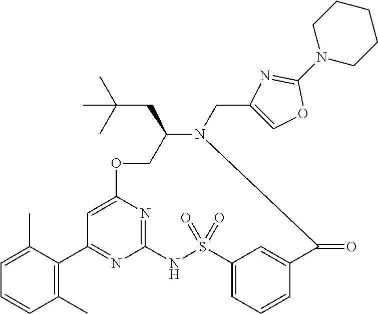 [SMILES: Cc1cccc(C)c1-c1cc2nc(n1)NS(=O)(=O)c1cccc(c1)C(=O)N(Cc1coc(N3CCCCC3)n1)[C@H](CC(C)(C)C)CO2] | 4.59 | 658.294 | 659.3 | Y |
| 1379 | 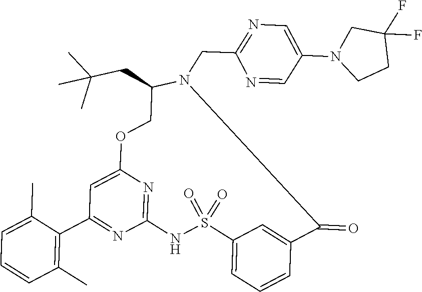 [SMILES: Cc1cccc(C)c1-c1cc2nc(n1)NS(=O)(=O)c1cccc(c1)C(=O)N(Cc1ncc(N3CCC(F)(F)C3)cn1)[C@H](CC(C)(C)C)CO2] | 1.81 | 691.275 | 692.6 | A |
| 1380 | 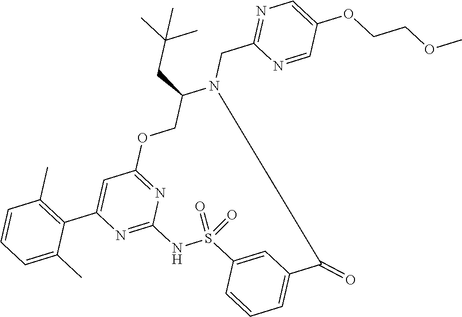 [SMILES: COCCOc1cnc(CN2C(=O)c3cccc(c3)S(=O)(=O)Nc3nc(cc(-c4c(C)cccc4C)n3)OC[C@H]2CC(C)(C)C)nc1] | 1.69 | 660.273 | 661.5 | D(30- 99% gradient) |
| 1381 | 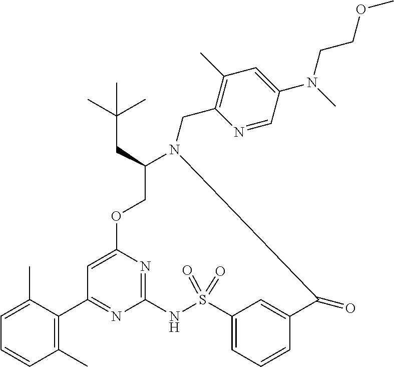 [SMILES: COCCN(C)c1cnc(CN2C(=O)c3cccc(c3)S(=O)(=O)Nc3nc(cc(-c4c(C)cccc4C)n3)OC[C@H]2CC(C)(C)C)c(C)c1] | 1.42 | 686.325 | 687.3 | A |
| 1382 | 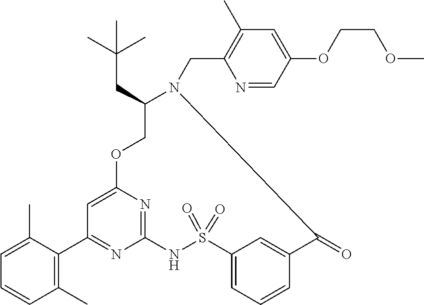 [SMILES: COCCOc1cnc(CN2C(=O)c3cccc(c3)S(=O)(=O)Nc3nc(cc(-c4c(C)cccc4C)n3)OC[C@H]2CC(C)(C)C)c(C)c1] | 1.77 | 673.293 | 674.6 | A |
| 1383 | 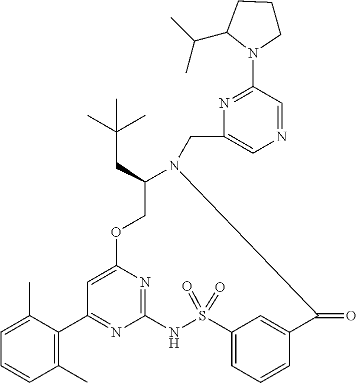 [SMILES: Cc1cccc(C)c1-c1cc2nc(n1)NS(=O)(=O)c1cccc(c1)C(=O)N(Cc1cncc(N3CCCC3C(C)C)n1)[C@H](CC(C)(C)C)CO2] | 1.63 | 697.341 | 698.4 | A |
| 1384 | 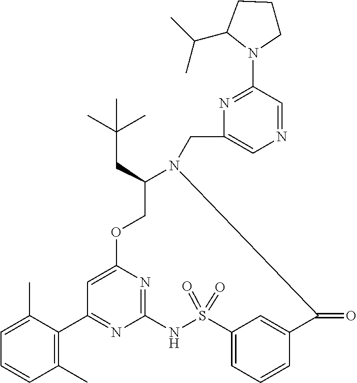 [SMILES: Cc1cccc(C)c1-c1cc2nc(n1)NS(=O)(=O)c1cccc(c1)C(=O)N(Cc1cncc(N3CCCC3C(C)C)n1)[C@H](CC(C)(C)C)CO2] | 1.63 | 697.341 | 698.5 | A |
| 1385 | 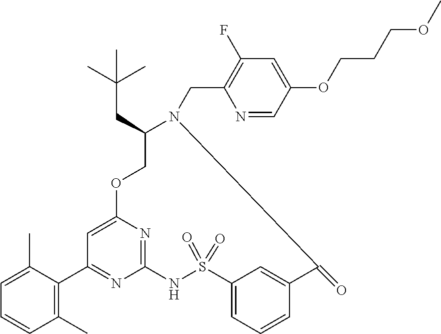 [SMILES: COCCCOc1cnc(CN2C(=O)c3cccc(c3)S(=O)(=O)Nc3nc(cc(-c4c(C)cccc4C)n3)OC[C@H]2CC(C)(C)C)c(F)c1] | 2.03 | 691.284 | 692.5 | A |
| 1386 | 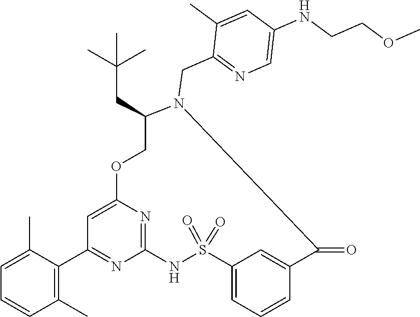 [SMILES: COCCNc1cnc(CN2C(=O)c3cccc(c3)S(=O)(=O)Nc3nc(cc(-c4c(C)cccc4C)n3)OC[C@H]2CC(C)(C)C)c(C)c1] | 1.44 | 672.309 | 673.5 | A |
| 1387 | 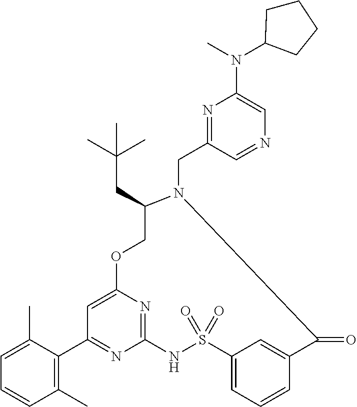 [SMILES: Cc1cccc(C)c1-c1cc2nc(n1)NS(=O)(=O)c1cccc(c1)C(=O)N(Cc1cncc(N(C)C3CCCC3)n1)[C@H](CC(C)(C)C)CO2] | 1.86 | 683.325 | 684.6 | A |
| 1388 | 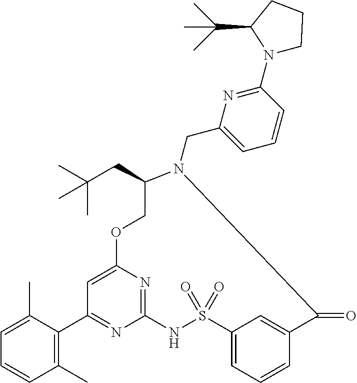 [SMILES: Cc1cccc(C)c1-c1cc2nc(n1)NS(=O)(=O)c1cccc(c1)C(=O)N(Cc1cccc(N3CCC[C@@H]3C(C)(C)C)n1)[C@H](CC(C)(C)C)CO2] | 1.65 | 710.361 | 711.5 | A |
| 1389 | 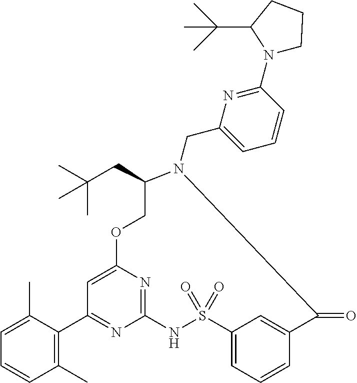 [SMILES: Cc1cccc(C)c1-c1cc2nc(n1)NS(=O)(=O)c1cccc(c1)C(=O)N(Cc1cccc(N3CCCC3C(C)(C)C)n1)[C@H](CC(C)(C)C)CO2] | 1.63 | 710.361 | 711.5 | A |
| 1390 | 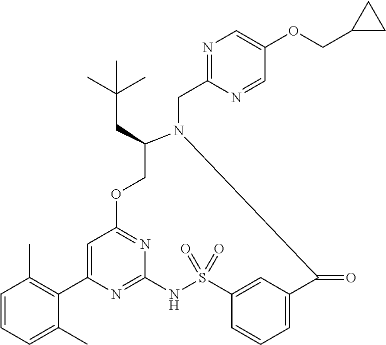 [SMILES: Cc1cccc(C)c1-c1cc2nc(n1)NS(=O)(=O)c1cccc(c1)C(=O)N(Cc1ncc(OCC3CC3)cn1)[C@H](CC(C)(C)C)CO2] | 1.88 | 656.278 | 657.2 | A |
| 1391 | 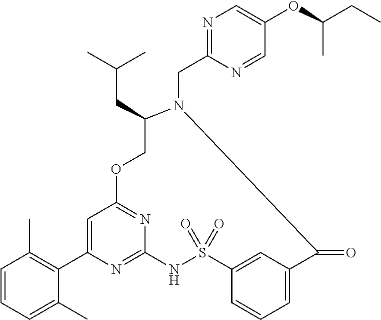 [SMILES: CC[C@@H](C)Oc1cnc(CN2C(=O)c3cccc(c3)S(=O)(=O)Nc3nc(cc(-c4c(C)cccc4C)n3)OC[C@H]2CC(C)C)nc1] | 1.91 | 644.278 | 645.5 | A |
| 1392 | 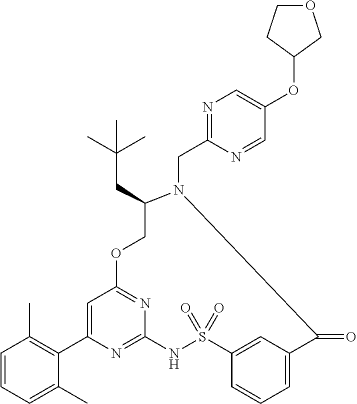 [SMILES: Cc1cccc(C)c1-c1cc2nc(n1)NS(=O)(=O)c1cccc(c1)C(=O)N(Cc1ncc(OC3CCOC3)cn1)[C@H](CC(C)(C)C)CO2] | 1.69 | 672.273 | 673.2 | A |
| 1393 | 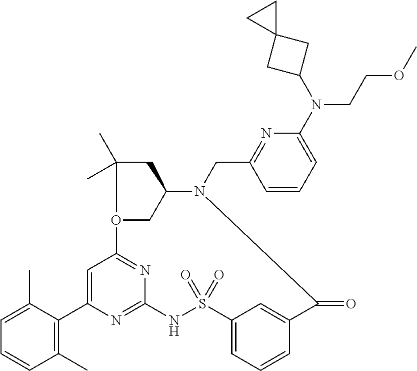 | 1.68 | 738.356 | 739.9 | A |
| 1394 | 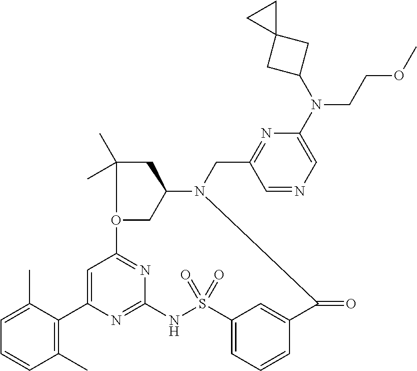 | 1.97 | 739.352 | 740.8 | A |
| 1395 | 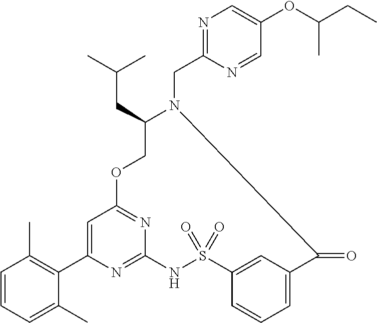 [SMILES: CCC(C)Oc1cnc(CN2C(=O)c3cccc(c3)S(=O)(=O)Nc3nc(cc(-c4c(C)cccc4C)n3)OC[C@H]2CC(C)C)nc1] | 1.91 | 644.278 | 645.7 | A |
| 1396 | 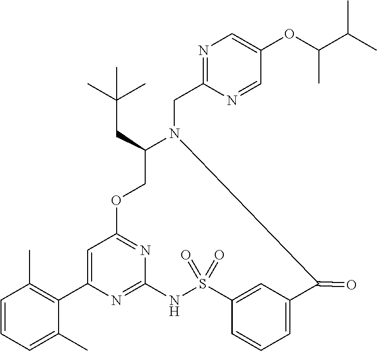 [SMILES: Cc1cccc(C)c1-c1cc2nc(n1)NS(=O)(=O)c1cccc(c1)C(=O)N(Cc1ncc(OC(C)C(C)C)cn1)[C@H](CC(C)(C)C)CO2] | 1.99 | 658.294 | 659.2 | A |
| 1397 | 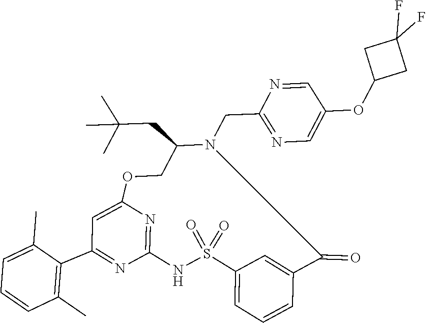 [SMILES: Cc1cccc(C)c1-c1cc2nc(n1)NS(=O)(=O)c1cccc(c1)C(=O)N(Cc1ncc(OC3CC(F)(F)C3)cn1)[C@H](CC(C)(C)C)CO2] | 1.87 | 692.259 | 693.2 | A |
| 1398 | 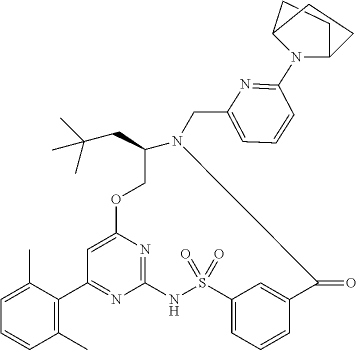 [SMILES: Cc1cccc(C)c1-c1cc2nc(n1)NS(=O)(=O)c1cccc(c1)C(=O)N(Cc1cccc(N3C4CCC3CC4)n1)[C@H](CC(C)(C)C)CO2] | 1.55 | 680.314 | 681.7 | A |
| 1399 | 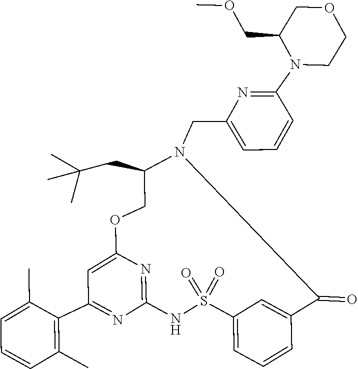 [SMILES: COC[C@H]1COCCN1c1cccc(CN2C(=O)c3cccc(c3)S(=O)(=O)Nc3nc(cc(-c4c(C)cccc4C)n3)OC[C@H]2CC(C)(C)C)n1] | 1.63 | 714.32 | 715.6 | A |
| 1400 | 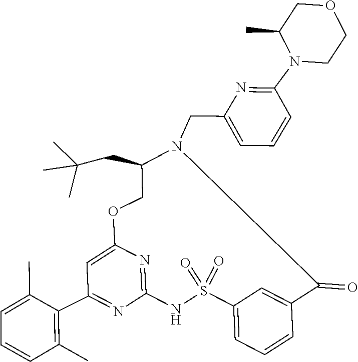 [SMILES: Cc1cccc(C)c1-c1cc2nc(n1)NS(=O)(=O)c1cccc(c1)C(=O)N(Cc1cccc(N3CCOC[C@@H]3C)n1)[C@H](CC(C)(C)C)CO2] | 1.63 | 684.309 | 685.7 | A |
| 1401 | 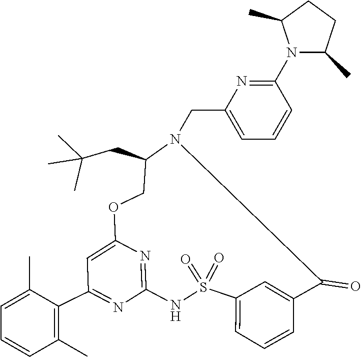 [SMILES: Cc1cccc(C)c1-c1cc2nc(n1)NS(=O)(=O)c1cccc(c1)C(=O)N(Cc1cccc(N3[C@H](C)CC[C@@H]3C)n1)[C@H](CC(C)(C)C)CO2] | 1.47 | 682.33 | 683.6 | A |
| 1402 | 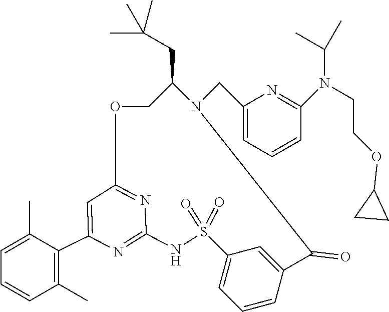 [SMILES: Cc1cccc(C)c1-c1cc2nc(n1)NS(=O)(=O)c1cccc(c1)C(=O)N(Cc1cccc(N(CCOC3CC3)C(C)C)n1)[C@H](CC(C)(C)C)CO2] | 2.45 | 726.356 | 727.6 | W |
| 1403 | 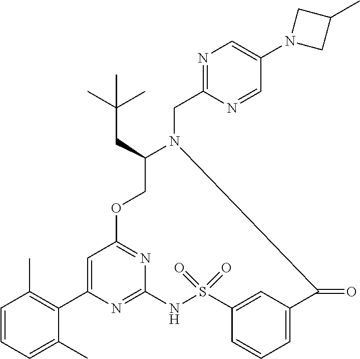 [SMILES: Cc1cccc(C)c1-c1cc2nc(n1)NS(=O)(=O)c1cccc(c1)C(=O)N(Cc1ncc(N3CC(C)C3)cn1)[C@H](CC(C)(C)C)CO2] | 1.58 | 655.294 | 656.44 | A |
| 1404 | 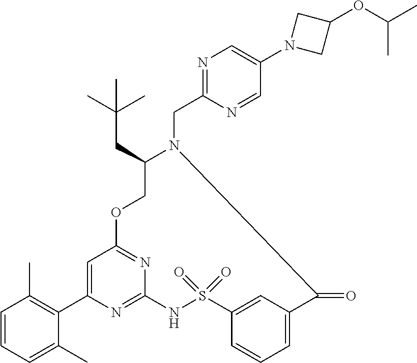 [SMILES: Cc1cccc(C)c1-c1cc2nc(n1)NS(=O)(=O)c1cccc(c1)C(=O)N(Cc1ncc(N3CC(OC(C)C)C3)cn1)[C@H](CC(C)(C)C)CO2] | 1.61 | 699.32 | 700.51 | A |
| 1405 | 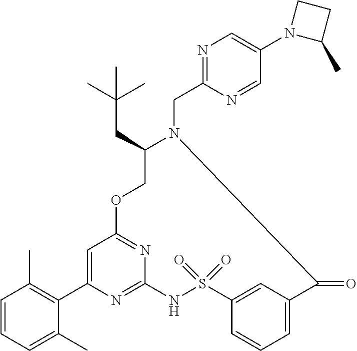 [SMILES: Cc1cccc(C)c1-c1cc2nc(n1)NS(=O)(=O)c1cccc(c1)C(=O)N(Cc1ncc(N3CC[C@H]3C)cn1)[C@H](CC(C)(C)C)CO2] | 1.58 | 655.294 | 656.4 | A |
| 1406 | 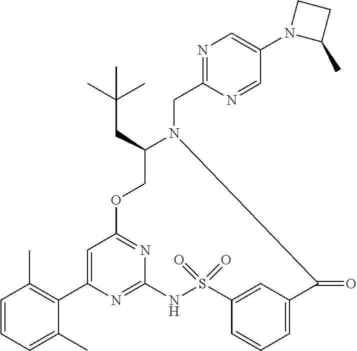 [SMILES: Cc1cccc(C)c1-c1cc2nc(n1)NS(=O)(=O)c1cccc(c1)C(=O)N(Cc1ncc(N3CC[C@H]3C)cn1)[C@H](CC(C)(C)C)CO2] | 1.57 | 655.294 | 656.5 | A |
| 1407 | 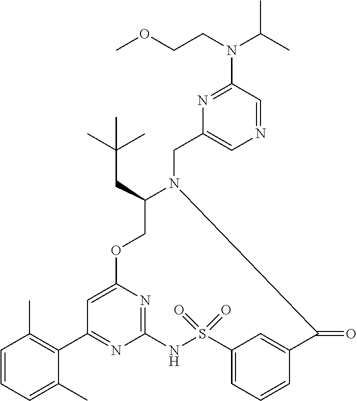 [SMILES: COCCN(c1cncc(CN2C(=O)c3cccc(c3)S(=O)(=O)Nc3nc(cc(-c4c(C)cccc4C)n3)OC[C@H]2CC(C)(C)C)n1)C(C)C] | 1.55 | 701.336 | 702.51 | A |
| 1408 | 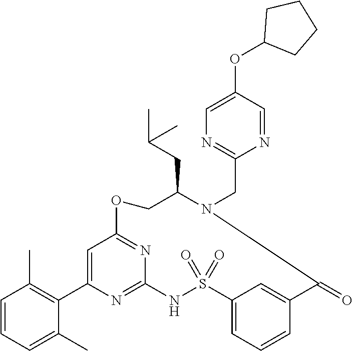 [SMILES: Cc1cccc(C)c1-c1cc2nc(n1)NS(=O)(=O)c1cccc(c1)C(=O)N(Cc1ncc(OC3CCCC3)cn1)[C@H](CC(C)C)CO2] | 1.96 | 656.278 | 657.7 | A |
| 1409 | 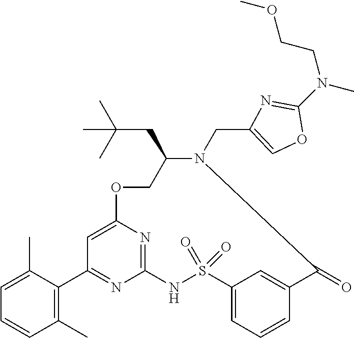 [SMILES: COCCN(C)c1nc(CN2C(=O)c3cccc(c3)S(=O)(=O)Nc3nc(cc(-c4c(C)cccc4C)n3)OC[C@H]2CC(C)(C)C)co1] | 4.12 | 662.289 | 663.3 | Y |
| 1410 | 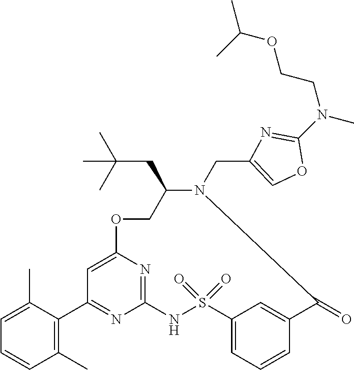 [SMILES: Cc1cccc(C)c1-c1cc2nc(n1)NS(=O)(=O)c1cccc(c1)C(=O)N(Cc1coc(N(C)CCOC(C)C)n1)[C@H](CC(C)(C)C)CO2] | 4.48 | 690.32 | 691.4 | Y |
| 1411 | 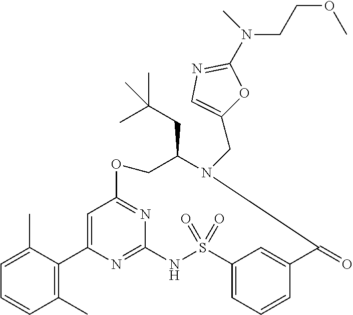 [SMILES: COCCN(C)c1ncc(CN2C(=O)c3cccc(c3)S(=O)(=O)Nc3nc(cc(-c4c(C)cccc4C)n3)OC[C@H]2CC(C)(C)C)o1] | 3.34 | 662.289 | 663.3 | Y |
| 1303 | 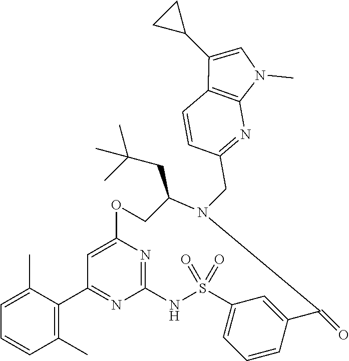 [SMILES: Cc1cccc(C)c1-c1cc2nc(n1)NS(=O)(=O)c1cccc(c1)C(=O)N(Cc1ccc3c(C4CC4)cn(C)c3n1)[C@H](CC(C)(C)C)CO2] | 4.97 | 678.299 | 679.3 | Y |
| 1412 | 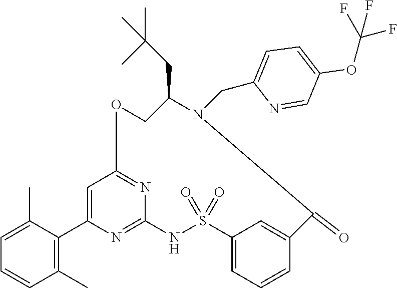 [SMILES: Cc1cccc(C)c1-c1cc2nc(n1)NS(=O)(=O)c1cccc(c1)C(=O)N(Cc1ccc(OC(F)(F)F)cn1)[C@H](CC(C)(C)C)CO2] | 4.75 | 669.223 | 670.3 | Y |
| 1413 | 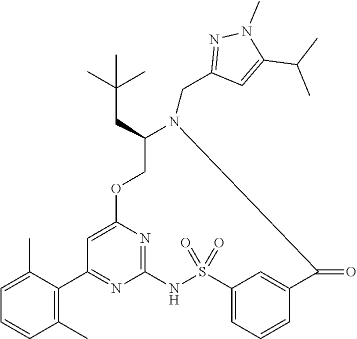 [SMILES: Cc1cccc(C)c1-c1cc2nc(n1)NS(=O)(=O)c1cccc(c1)C(=O)N(Cc1cc(C(C)C)n(C)n1)[C@H](CC(C)(C)C)CO2] | 1.85 | 630.299 | 631.6 | A |
| 1414 | 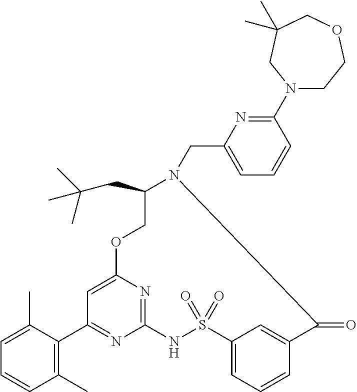 [SMILES: Cc1cccc(C)c1-c1cc2nc(n1)NS(=O)(=O)c1cccc(c1)C(=O)N(Cc1cccc(N3CCOCC(C)(C)C3)n1)[C@H](CC(C)(C)C)CO2] | 1.54 | 712.341 | 713.7 | A |
| 1415 | 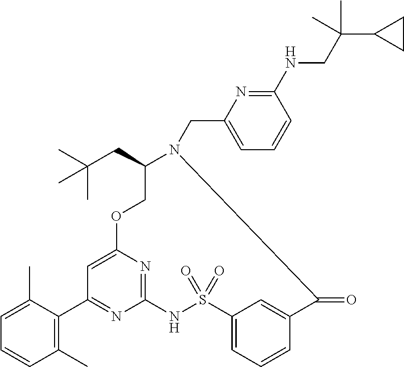 [SMILES: Cc1cccc(C)c1-c1cc2nc(n1)NS(=O)(=O)c1cccc(c1)C(=O)N(Cc1cccc(NCC(C)(C)C3CC3)n1)[C@H](CC(C)(C)C)CO2] | 1.56 | 696.346 | 697.8 | A |
| 1416 | 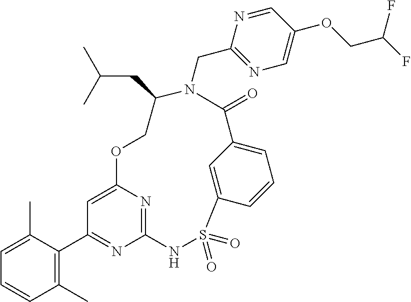 [SMILES: Cc1cccc(C)c1-c1cc2nc(n1)NS(=O)(=O)c1cccc(c1)C(=O)N(Cc1ncc(OCC(F)F)cn1)[C@H](CC(C)C)CO2] | 1.79 | 666.244 | 667.2 | A |
| 1417 | 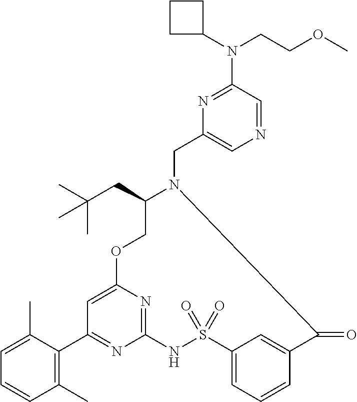 [SMILES: COCCN(c1cncc(CN2C(=O)c3cccc(c3)S(=O)(=O)Nc3nc(cc(-c4c(C)cccc4C)n3)OC[C@H]2CC(C)(C)C)n1)C1CCC1] | 1.89 | 713.336 | 714.5 | A |
| 1418 | 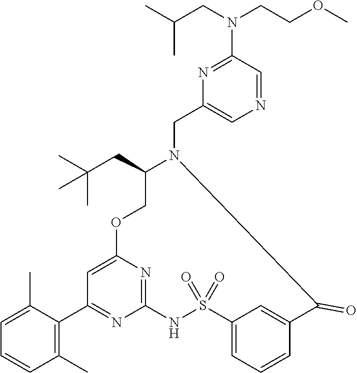 [SMILES: COCCN(CC(C)C)c1cncc(CN2C(=O)c3cccc(c3)S(=O)(=O)Nc3nc(cc(-c4c(C)cccc4C)n3)OC[C@H]2CC(C)(C)C)n1] | 1.9 | 715.352 | 716.5 | A |
| 1419 | 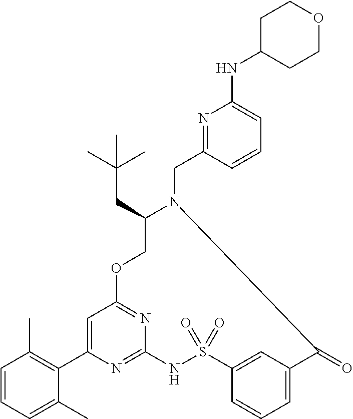 [SMILES: Cc1cccc(C)c1-c1cc2nc(n1)NS(=O)(=O)c1cccc(c1)C(=O)N(Cc1cccc(NC3CCOCC3)n1)[C@H](CC(C)(C)C)CO2] | 1.34 | 684.309 | 685.6 | A |
| 1420 | 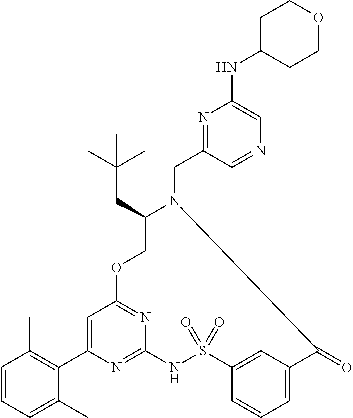 [SMILES: Cc1cccc(C)c1-c1cc2nc(n1)NS(=O)(=O)c1cccc(c1)C(=O)N(Cc1cncc(NC3CCOCC3)n1)[C@H](CC(C)(C)C)CO2] | 1.61 | 685.305 | 686.6 | A |
| 1305 | 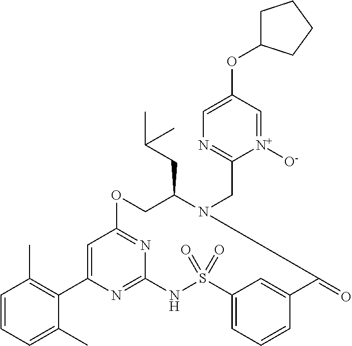 [SMILES: Cc1cccc(C)c1-c1cc2nc(n1)NS(=O)(=O)c1cccc(c1)C(=O)N(Cc1ncc(OC3CCCC3)c[n+]1[O-])[C@H](CC(C)C)CO2] | 1.73 | 672.273 | 673.5 | A |
| 1421 | 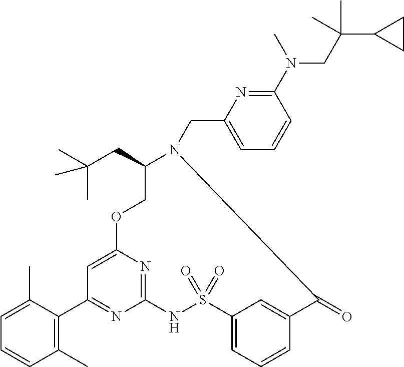 [SMILES: Cc1cccc(C)c1-c1cc2nc(n1)NS(=O)(=O)c1cccc(c1)C(=O)N(Cc1cccc(N(C)CC(C)(C)C3CC3)n1)[C@H](CC(C)(C)C)CO2] | 1.65 | 710.361 | 711.6 | A |
| 1422 | 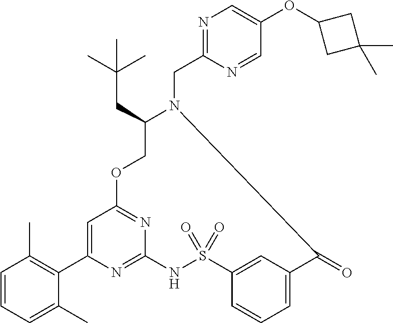 [SMILES: Cc1cccc(C)c1-c1cc2nc(n1)NS(=O)(=O)c1cccc(c1)C(=O)N(Cc1ncc(OC3CC(C)(C)C3)cn1)[C@H](CC(C)(C)C)CO2] | 2.1 | 684.309 | 685.2 | A |
| 1423 | 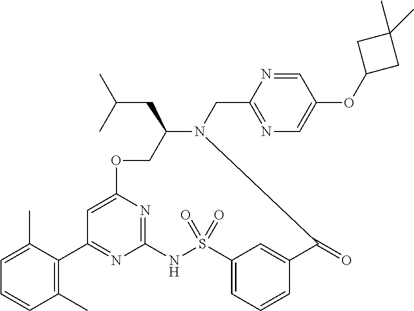 [SMILES: Cc1cccc(C)c1-c1cc2nc(n1)NS(=O)(=O)c1cccc(c1)C(=O)N(Cc1ncc(OC3CC(C)(C)C3)cn1)[C@H](CC(C)C)CO2] | 0.81 | 670.294 | 671.2 | D |
| 1424 | 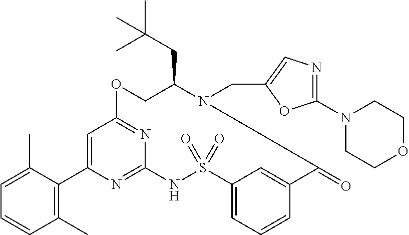 [SMILES: Cc1cccc(C)c1-c1cc2nc(n1)NS(=O)(=O)c1cccc(c1)C(=O)N(Cc1cnc(N3CCOCC3)o1)[C@H](CC(C)(C)C)CO2] | 3.7 | 660.273 | 661.3 | Y |
| 1425 | 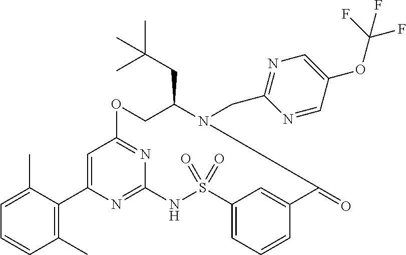 [SMILES: Cc1cccc(C)c1-c1cc2nc(n1)NS(=O)(=O)c1cccc(c1)C(=O)N(Cc1ncc(OC(F)(F)F)cn1)[C@H](CC(C)(C)C)CO2] | 4.42 | 670.219 | 671.2 | Y |
| 1297 | 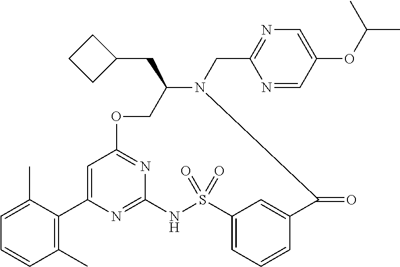 [SMILES: Cc1cccc(C)c1-c1cc2nc(n1)NS(=O)(=O)c1cccc(c1)C(=O)N(Cc1ncc(OC(C)C)cn1)[C@H](CC1CCC1)CO2] | 1.93 | 642.262 | 643.62 | A |
| 1426 | 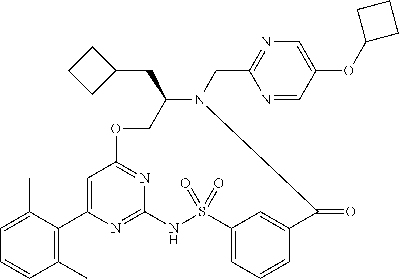 [SMILES: Cc1cccc(C)c1-c1cc2nc(n1)NS(=O)(=O)c1cccc(c1)C(=O)N(Cc1ncc(OC3CCC3)cn1)[C@H](CC1CCC1)CO2] | 1.92 | 654.262 | 655.5 | A |
| 1427 | 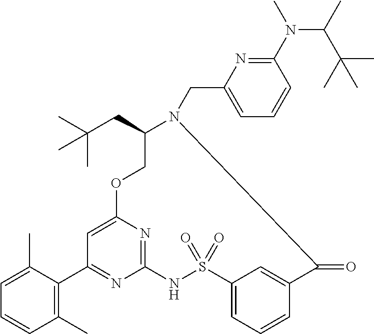 [SMILES: Cc1cccc(C)c1-c1cc2nc(n1)NS(=O)(=O)c1cccc(c1)C(=O)N(Cc1cccc(N(C)C(C)C(C)(C)C)n1)[C@H](CC(C)(C)C)CO2] | 1.61 | 698.361 | 699.7 | A |
| 1428 | 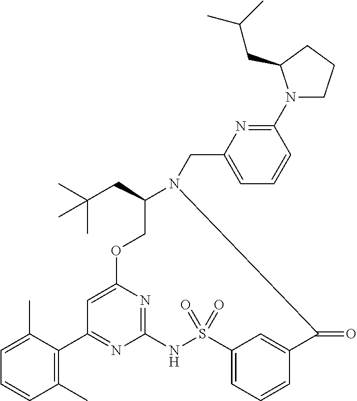 [SMILES: Cc1cccc(C)c1-c1cc2nc(n1)NS(=O)(=O)c1cccc(c1)C(=O)N(Cc1cccc(N3CCC[C@@H]3CC(C)C)n1)[C@H](CC(C)(C)C)CO2] | 1.64 | 710.361 | 711.8 | A |
| 1429 | 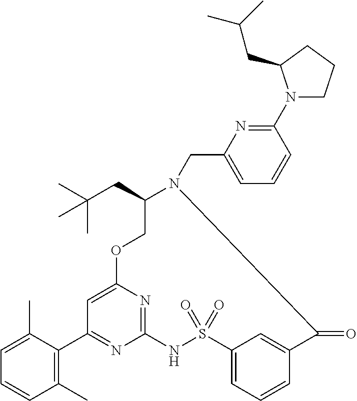 [SMILES: Cc1cccc(C)c1-c1cc2nc(n1)NS(=O)(=O)c1cccc(c1)C(=O)N(Cc1cccc(N3CCC[C@@H]3CC(C)C)n1)[C@H](CC(C)(C)C)CO2] | 1.64 | 710.361 | 711.7 | A |
| 1430 | 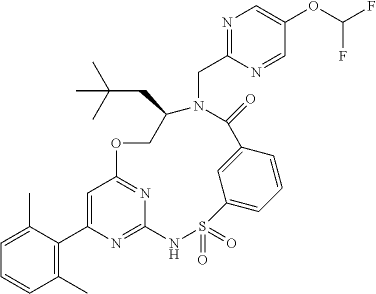 [SMILES: Cc1cccc(C)c1-c1cc2nc(n1)NS(=O)(=O)c1cccc(c1)C(=O)N(Cc1ncc(OC(F)F)cn1)[C@H](CC(C)(C)C)CO2] | 1.8 | 652.228 | 653.2 | A |
| 1431 | 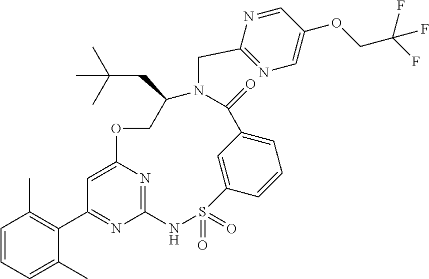 [SMILES: Cc1cccc(C)c1-c1cc2nc(n1)NS(=O)(=O)c1cccc(c1)C(=O)N(Cc1ncc(OCC(F)(F)F)cn1)[C@H](CC(C)(C)C)CO2] | 1.87 | 684.234 | 685.2 | A |
| 1432 | 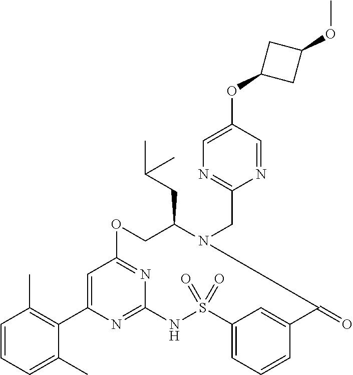 [SMILES: CO[C@H]1C[C@@H](Oc2cnc(CN3C(=O)c4cccc(c4)S(=O)(=O)Nc4nc(cc(-c5c(C)cccc5C)n4)OC[C@H]3CC(C)C)nc2)C1] | 1.75 | 672.273 | 673.2 | A |
| 1433 | 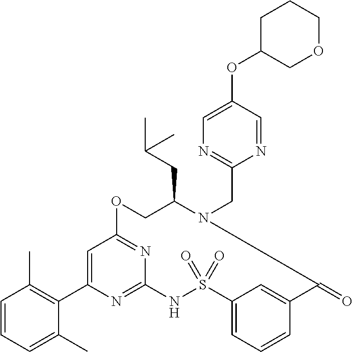 [SMILES: Cc1cccc(C)c1-c1cc2nc(n1)NS(=O)(=O)c1cccc(c1)C(=O)N(Cc1ncc(OC3CCCOC3)cn1)[C@H](CC(C)C)CO2] | 1.71 | 672.273 | 673.1 | A |
| 1434 | 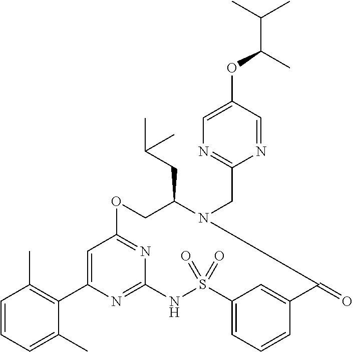 [SMILES: Cc1cccc(C)c1-c1cc2nc(n1)NS(=O)(=O)c1cccc(c1)C(=O)N(Cc1ncc(O[C@H](C)C(C)C)cn1)[C@H](CC(C)C)CO2] | 2.02 | 658.294 | 659.255 | A |
| 1435 | 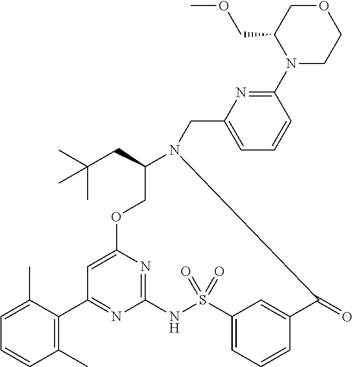 [SMILES: COC[C@@H]1COCCN1c1cccc(CN2C(=O)c3cccc(c3)S(=O)(=O)Nc3nc(cc(-c4c(C)cccc4C)n3)OC[C@H]2CC(C)(C)C)n1] | 1.63 | 714.32 | 715.6 | A |
| 1436 | 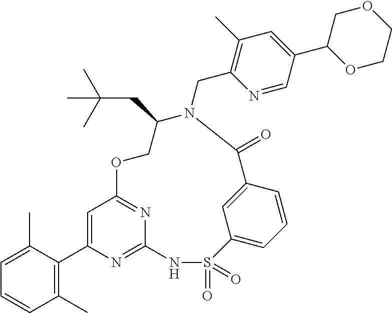 [SMILES: Cc1cc(C2COCCO2)cnc1CN1C(=O)c2cccc(c2)S(=O)(=O)Nc2nc(cc(-c3c(C)cccc3C)n2)OC[C@H]1CC(C)(C)C] | 1.51 | 685.293 | 686.5 | A |
| 1437 | 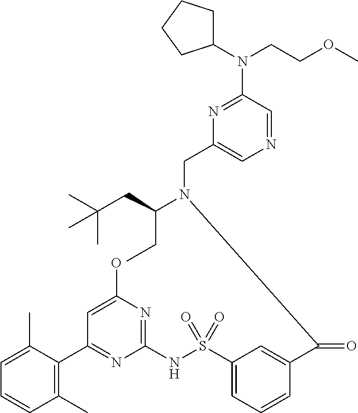 [SMILES: CC[C@@H](CC(C)(C)C)N(Cc1cncc(N(CCOC)C2CCCC2)n1)C(=O)c1cccc(S(=O)(=O)Nc2nc(O)cc(-c3c(C)cccc3C)n2)c1] | 1.92 | 727.352 | 728.5 | A |
| 1438 | 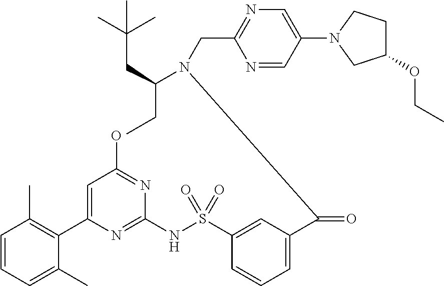 [SMILES: CCO[C@H]1CCN(c2cnc(CN(C(=O)c3cccc(S(=O)(=O)Nc4nc(O)cc(-c5c(C)cccc5C)n4)c3)[C@@H](CC)CC(C)(C)C)nc2)C1] | 1.77 | 699.32 | 700.5 | A |
| 1439 | 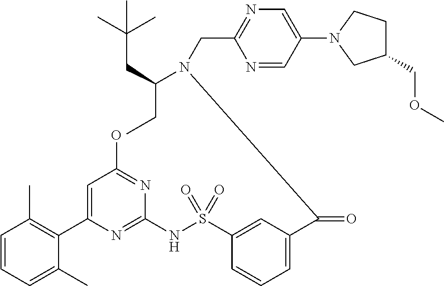 [SMILES: CC[C@@H](CC(C)(C)C)N(Cc1ncc(N2CC[C@H](COC)C2)cn1)C(=O)c1cccc(S(=O)(=O)Nc2nc(O)cc(-c3c(C)cccc3C)n2)c1] | 1.79 | 699.32 | 700.5 | A |
| 1440 | 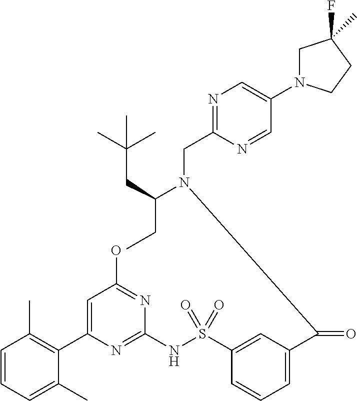 [SMILES: CC[C@@H](CC(C)(C)C)N(Cc1ncc(N2CC[C@](C)(F)C2)cn1)C(=O)c1cccc(S(=O)(=O)Nc2nc(O)cc(-c3c(C)cccc3C)n2)c1] | 1.5 | 687.3 | 688.1 | A |
| 1441 | 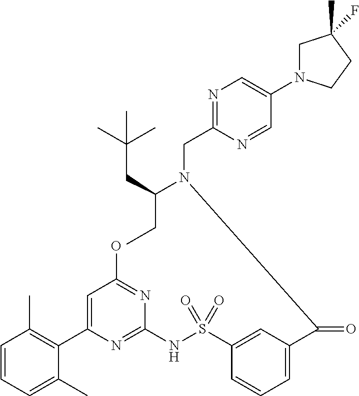 [SMILES: CC[C@@H](CC(C)(C)C)N(Cc1ncc(N2CC[C@@](C)(F)C2)cn1)C(=O)c1cccc(S(=O)(=O)Nc2nc(O)cc(-c3c(C)cccc3C)n2)c1] | 1.5 | 687.3 | 688.4 | A |
| 1442 | 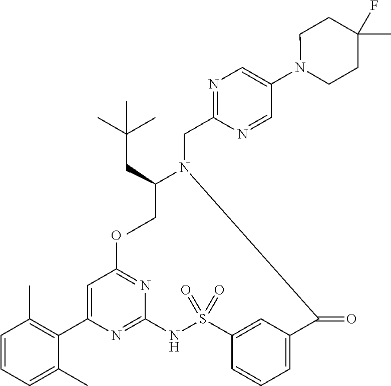 [SMILES: CC[C@@H](CC(C)(C)C)N(Cc1ncc(N2CCC(C)(F)CC2)cn1)C(=O)c1cccc(S(=O)(=O)Nc2nc(O)cc(-c3c(C)cccc3C)n2)c1] | 1.56 | 701.316 | 702.4 | A |
| 1443 | 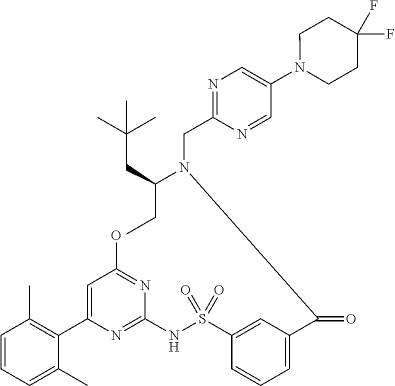 [SMILES: CC[C@@H](CC(C)(C)C)N(Cc1ncc(N2CCC(F)(F)CC2)cn1)C(=O)c1cccc(S(=O)(=O)Nc2nc(O)cc(-c3c(C)cccc3C)n2)c1] | 1.54 | 705.291 | 706.4 | A |
| 1444 | 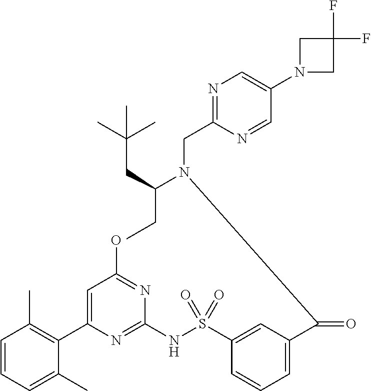 [SMILES: CC[C@@H](CC(C)(C)C)N(Cc1ncc(N2CC(F)(F)C2)cn1)C(=O)c1cccc(S(=O)(=O)Nc2nc(O)cc(-c3c(C)cccc3C)n2)c1] | 1.49 | 677.26 | 678.4 | A |
| 1445 | 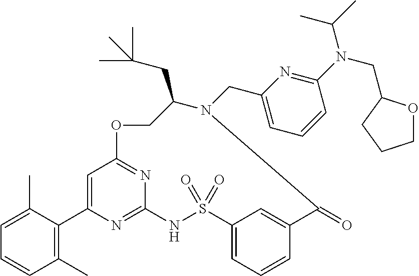 [SMILES: Cc1cccc(C)c1-c1cc2nc(n1)NS(=O)(=O)c1cccc(c1)C(=O)N(Cc1cccc(N(CC3CCCO3)C(C)C)n1)[C@H](CC(C)(C)C)CO2] | 2.46 | 726.356 | 727.6 | W |
| 1446 | 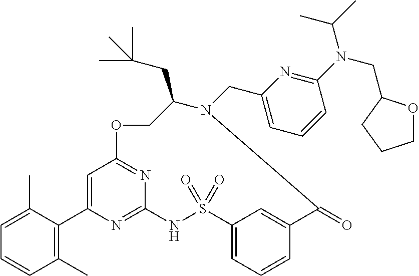 [SMILES: Cc1cccc(C)c1-c1cc2nc(n1)NS(=O)(=O)c1cccc(c1)C(=O)N(Cc1cccc(N(CC3CCCO3)C(C)C)n1)[C@H](CC(C)(C)C)CO2] | 2.52 | 726.356 | 727.6 | W |
| 1447 | 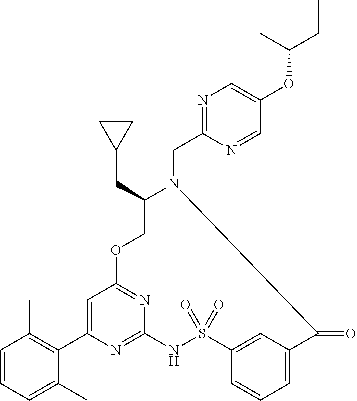 [SMILES: CC[C@@H](C)Oc1cnc(CN2C(=O)c3cccc(c3)S(=O)(=O)Nc3nc(cc(-c4c(C)cccc4C)n3)OC[C@H]2CC2CC2)nc1] | 1.84 | 642.262 | 643.2 | A |
| 1448 | 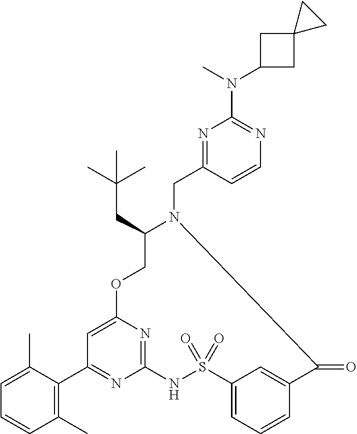 [SMILES: Cc1cccc(C)c1-c1cc2nc(n1)NS(=O)(=O)c1cccc(c1)C(=O)N(Cc1ccnc(N(C)C3CC4(CC4)C3)n1)[C@H](CC(C)(C)C)CO2] | 1.74 | 695.325 | 696.6 | A |
| 1449 | 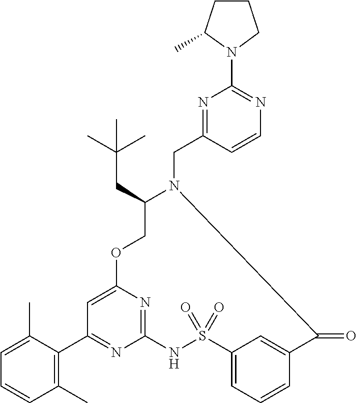 [SMILES: Cc1cccc(C)c1-c1cc2nc(n1)NS(=O)(=O)c1cccc(c1)C(=O)N(Cc1ccnc(N3CCC[C@H]3C)n1)[C@H](CC(C)(C)C)CO2] | 1.46 | 669.31 | 670.5 | A |
| 1450 | 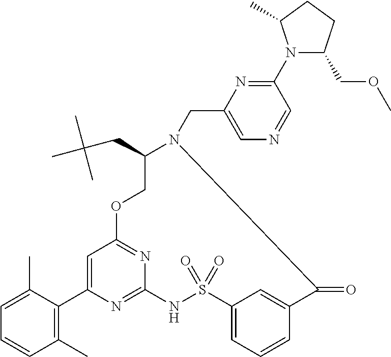 [SMILES: COC[C@H]1CC[C@@H](C)N1c1cncc(CN2C(=O)c3cccc(c3)S(=O)(=O)Nc3nc(cc(-c4c(C)cccc4C)n3)OC[C@H]2CC(C)(C)C)n1] | 1.82 | 713.336 | 714.6 | A |
| 1451 | 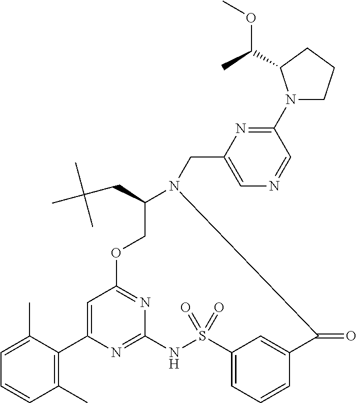 [SMILES: CO[C@H](C)[C@@H]1CCCN1c1cncc(CN2C(=O)c3cccc(c3)S(=O)(=O)Nc3nc(cc(-c4c(C)cccc4C)n3)OC[C@H]2CC(C)(C)C)n1] | 1.76 | 713.336 | 714.7 | A |
| 1452 | 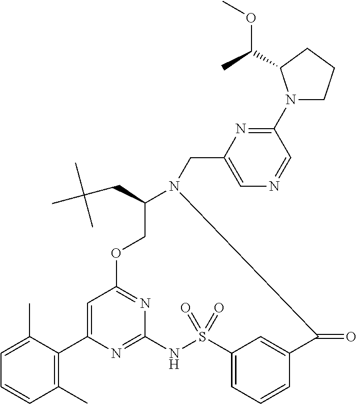 [SMILES: CO[C@H](C)[C@@H]1CCCN1c1cncc(CN2C(=O)c3cccc(c3)S(=O)(=O)Nc3nc(cc(-c4c(C)cccc4C)n3)OC[C@H]2CC(C)(C)C)n1] | 1.79 | 713.336 | 714.7 | A |
| 1453 | 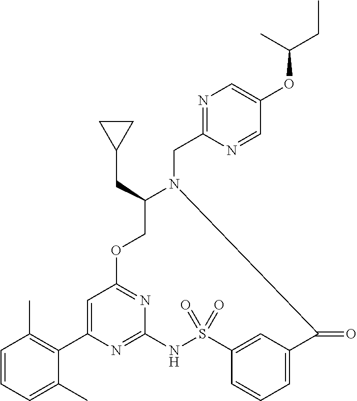 [SMILES: CC[C@H](C)Oc1cnc(CN2C(=O)c3cccc(c3)S(=O)(=O)Nc3nc(cc(-c4c(C)cccc4C)n3)OC[C@H]2CC2CC2)nc1] | 1.84 | 642.262 | 643.2 | A |
| 1454 | 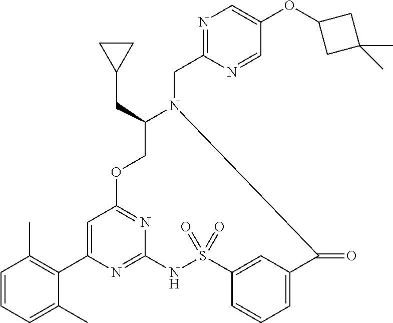 [SMILES: Cc1cccc(C)c1-c1cc2nc(n1)NS(=O)(=O)c1cccc(c1)C(=O)N(Cc1ncc(OC3CC(C)(C)C3)cn1)[C@H](CC1CC1)CO2] | 1.98 | 668.278 | 669.2 | A |
| 1455 | 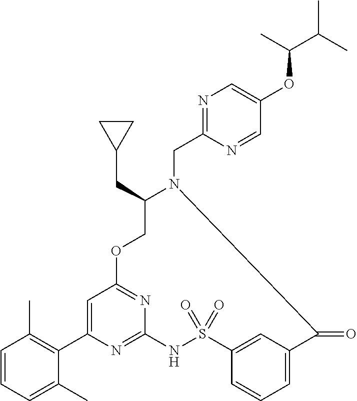 [SMILES: Cc1cccc(C)c1-c1cc2nc(n1)NS(=O)(=O)c1cccc(c1)C(=O)N(Cc1ncc(O[C@@H](C)C(C)C)cn1)[C@H](CC1CC1)CO2] | 1.94 | 656.278 | 657.2 | A |
| 1456 | 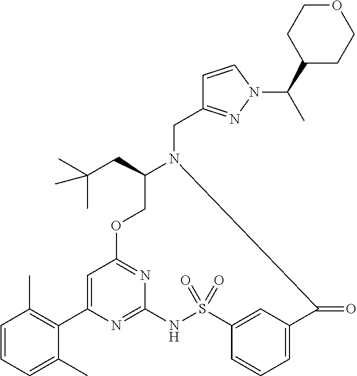 [SMILES: Cc1cccc(C)c1-c1cc2nc(n1)NS(=O)(=O)c1cccc(c1)C(=O)N(Cc1ccn([C@H](C)C3CCOCC3)n1)[C@H](CC(C)(C)C)CO2] | 4.38 | 686.325 | 687.3 | Y |
| 1457 | 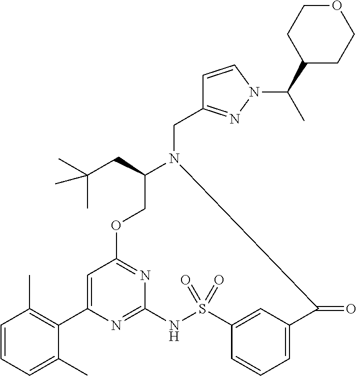 [SMILES: Cc1cccc(C)c1-c1cc2nc(n1)NS(=O)(=O)c1cccc(c1)C(=O)N(Cc1ccn([C@H](C)C3CCOCC3)n1)[C@H](CC(C)(C)C)CO2] | 4.35 | 686.325 | 687.3 | Y |
| 1458 | 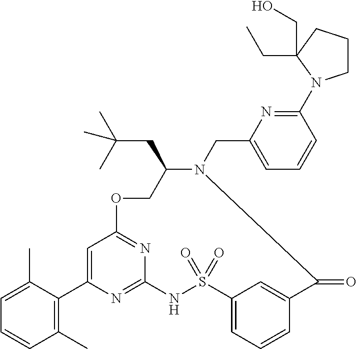 [SMILES: CCC1(CO)CCCN1c1cccc(CN2C(=O)c3cccc(c3)S(=O)(=O)Nc3nc(cc(-c4c(C)cccc4C)n3)OC[C@H]2CC(C)(C)C)n1] | 3.25 | 712.341 | 713.3 | Y |
| 1459 | 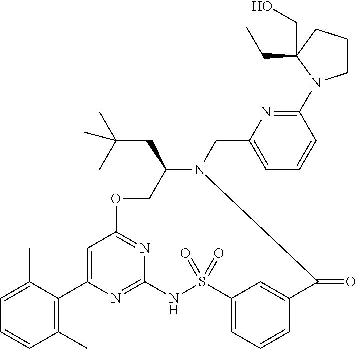 [SMILES: CC[C@@]1(CO)CCCN1c1cccc(CN2C(=O)c3cccc(c3)S(=O)(=O)Nc3nc(cc(-c4c(C)cccc4C)n3)OC[C@H]2CC(C)(C)C)n1] | 3.26 | 712.341 | 713.3 | Y |
| 1460 | 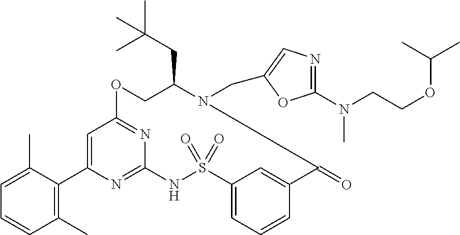 [SMILES: Cc1cccc(C)c1-c1cc2nc(n1)NS(=O)(=O)c1cccc(c1)C(=O)N(Cc1cnc(N(C)CCOC(C)C)o1)[C@H](CC(C)(C)C)CO2] | 3.06 | 690.32 | 691.3 | 1D |
| 1304 | 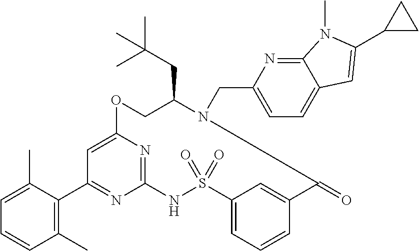 [SMILES: Cc1cccc(C)c1-c1cc2nc(n1)NS(=O)(=O)c1cccc(c1)C(=O)N(Cc1ccc3cc(C4CC4)n(C)c3n1)[C@H](CC(C)(C)C)CO2] | 4.96 | 678.299 | 679.3 | Y |
| 1461 | 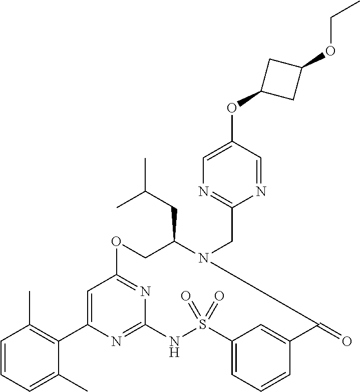 [SMILES: CCO[C@H]1C[C@@H](Oc2cnc(CN3C(=O)c4cccc(c4)S(=O)(=O)Nc4nc(cc(-c5c(C)cccc5C)n4)OC[C@H]3CC(C)C)nc2)C1] | 1.82 | 686.289 | 687.519 | A |
| 1462 | 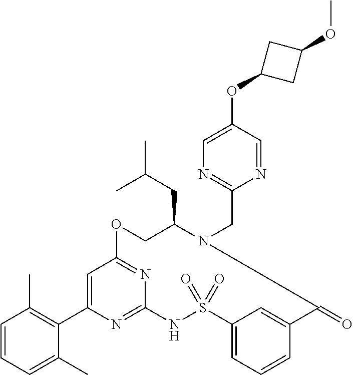 [SMILES: CO[C@H]1C[C@@H](Oc2cnc(CN3C(=O)c4cccc(c4)S(=O)(=O)Nc4nc(cc(-c5c(C)cccc5C)n4)OC[C@H]3CC(C)C)nc2)C1] | 1.75 | 672.273 | 673.544 | A |
| 1463 | 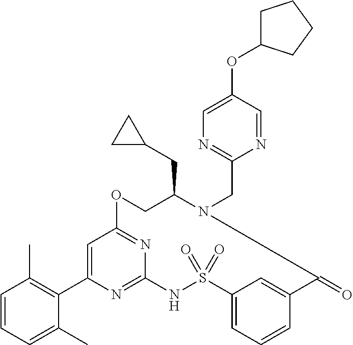 [SMILES: Cc1cccc(C)c1-c1cc2nc(n1)NS(=O)(=O)c1cccc(c1)C(=O)N(Cc1ncc(OC3CCCC3)cn1)[C@H](CC1CC1)CO2] | 1.86 | 654.262 | 655.442 | A |
| 1464 | 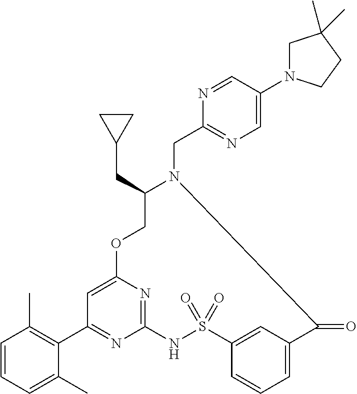 [SMILES: Cc1cccc(C)c1-c1cc2nc(n1)NS(=O)(=O)c1cccc(c1)C(=O)N(Cc1ncc(N3CCC(C)(C)C3)cn1)[C@H](CC1CC1)CO2] | 1.83 | 667.294 | 668.6 | A |
| 1465 | 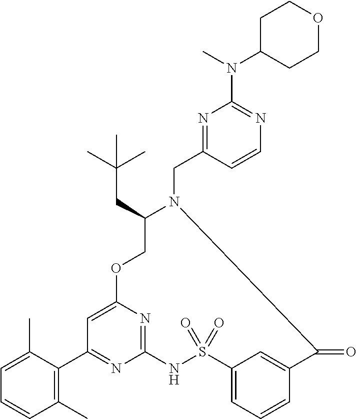 [SMILES: Cc1cccc(C)c1-c1cc2nc(n1)NS(=O)(=O)c1cccc(c1)C(=O)N(Cc1ccnc(N(C)C3CCOCC3)n1)[C@H](CC(C)(C)C)CO2] | 1.52 | 699.32 | 700.6 | A |
| 1466 | 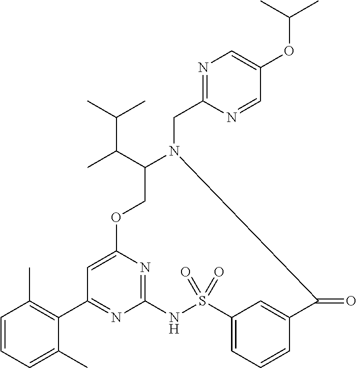 [SMILES: Cc1cccc(C)c1-c1cc2nc(n1)NS(=O)(=O)c1cccc(c1)C(=O)N(Cc1ncc(OC(C)C)cn1)C(C(C)C(C)C)CO2] | 1.89 | 644.278 | 645.5 | A |
| 1467 | 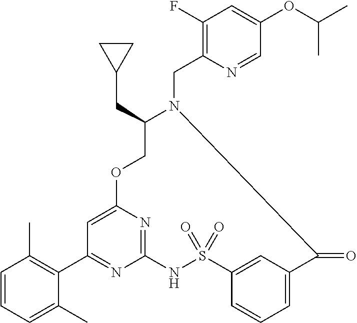 [SMILES: Cc1cccc(C)c1-c1cc2nc(n1)NS(=O)(=O)c1cccc(c1)C(=O)N(Cc1ncc(OC(C)C)cc1F)[C@H](CC1CC1)CO2] | 1.93 | 645.242 | 646.6 | A |
| 1468 | 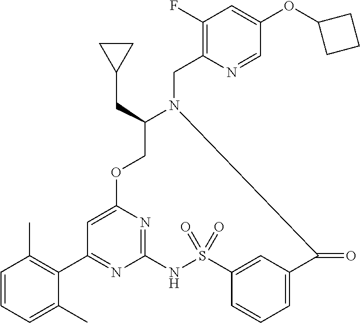 [SMILES: Cc1cccc(C)c1-c1cc2nc(n1)NS(=O)(=O)c1cccc(c1)C(=O)N(Cc1ncc(OC3CCC3)cc1F)[C@H](CC1CC1)CO2] | 1.99 | 657.242 | 658.5 | A |
| 1469 | 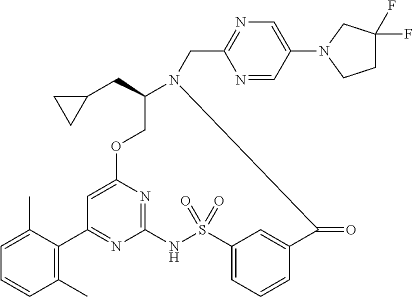 [SMILES: Cc1cccc(C)c1-c1cc2nc(n1)NS(=O)(=O)c1cccc(c1)C(=O)N(Cc1ncc(N3CCC(F)(F)C3)cn1)[C@H](CC1CC1)CO2] | 1.69 | 675.244 | 676.5 | A |
| 1470 | 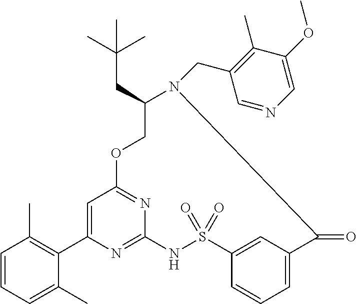 [SMILES: COc1cncc(CN2C(=O)c3cccc(c3)S(=O)(=O)Nc3nc(cc(-c4c(C)cccc4C)n3)OC[C@H]2CC(C)(C)C)c1C] | 1.2 | 629.267 | 630.4 | A |
| 1471 | 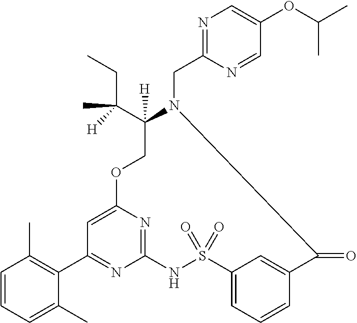 [SMILES: CC[C@H](C)[C@@H]1COc2cc(-c3c(C)cccc3C)nc(n2)NS(=O)(=O)c2cccc(c2)C(=O)N1Cc1ncc(OC(C)C)cn1] | 1.8 | 630.262 | 631.5 | A |
| 1472 | 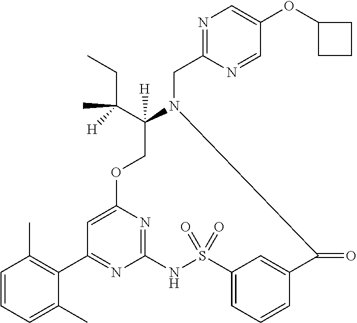 [SMILES: CC[C@H](C)[C@@H]1COc2cc(-c3c(C)cccc3C)nc(n2)NS(=O)(=O)c2cccc(c2)C(=O)N1Cc1ncc(OC2CCC2)cn1] | 1.86 | 642.262 | 643.5 | A |
| 1473 | 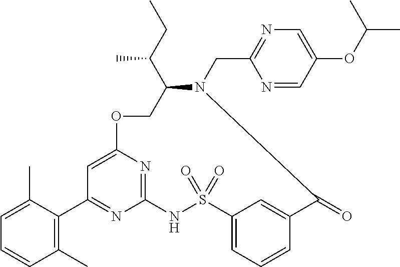 [SMILES: CC[C@@H](C)[C@@H]1COc2cc(-c3c(C)cccc3C)nc(n2)NS(=O)(=O)c2cccc(c2)C(=O)N1Cc1ncc(OC(C)C)cn1] | 1.8 | 630.262 | 631.45 | A |
| 1474 | 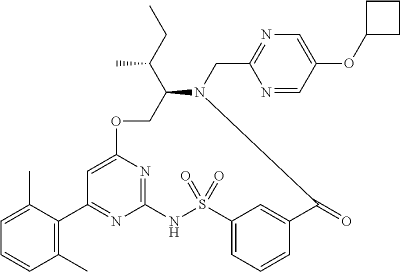 [SMILES: CC[C@@H](C)[C@@H]1COc2cc(-c3c(C)cccc3C)nc(n2)NS(=O)(=O)c2cccc(c2)C(=O)N1Cc1ncc(OC2CCC2)cn1] | 1.86 | 642.262 | 643.45 | A |
| 1475 | 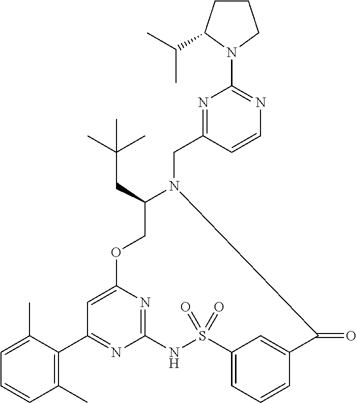 [SMILES: Cc1cccc(C)c1-c1cc2nc(n1)NS(=O)(=O)c1cccc(c1)C(=O)N(Cc1ccnc(N3CCC[C@H]3C(C)C)n1)[C@H](CC(C)(C)C)CO2] | 1.61 | 697.341 | 698.6 | A |
| 1476 | 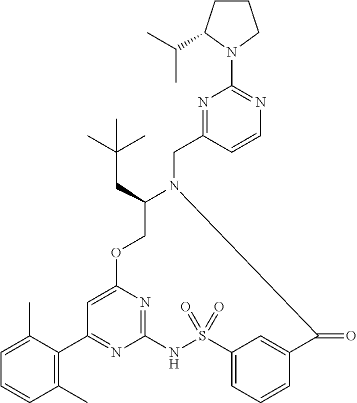 [SMILES: Cc1cccc(C)c1-c1cc2nc(n1)NS(=O)(=O)c1cccc(c1)C(=O)N(Cc1ccnc(N3CCC[C@H]3C(C)C)n1)[C@H](CC(C)(C)C)CO2] | 1.61 | 697.341 | 698.6 | A |
| 1477 | 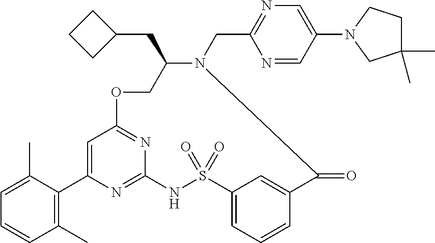 [SMILES: Cc1cccc(C)c1-c1cc2nc(n1)NS(=O)(=O)c1cccc(c1)C(=O)N(Cc1ncc(N3CCC(C)(C)C3)cn1)[C@H](CC1CCC1)CO2] | 1.93 | 681.31 | 682.6 | A |
| 1478 | 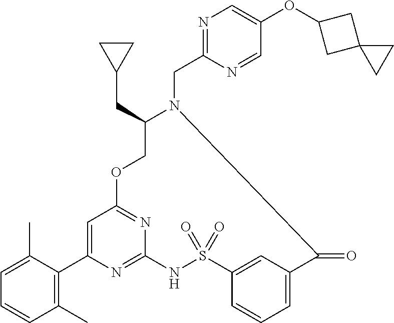 [SMILES: Cc1cccc(C)c1-c1cc2nc(n1)NS(=O)(=O)c1cccc(c1)C(=O)N(Cc1ncc(OC3CC4(CC4)C3)cn1)[C@H](CC1CC1)CO2] | 1.92 | 666.262 | 667.2 | A |
| 1479 | 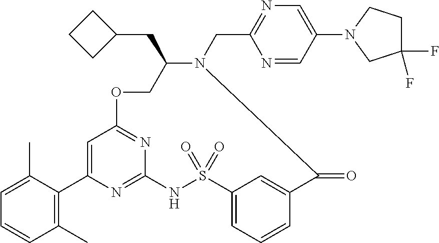 [SMILES: Cc1cccc(C)c1-c1cc2nc(n1)NS(=O)(=O)c1cccc(c1)C(=O)N(Cc1ncc(N3CCC(F)(F)C3)cn1)[C@H](CC1CCC1)CO2] | 1.79 | 689.26 | 690.7 | A |
| 1480 | 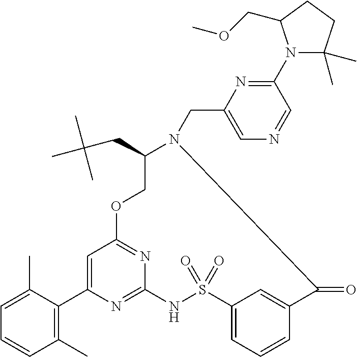 [SMILES: COCC1CCC(C)(C)N1c1cncc(CN2C(=O)c3cccc(c3)S(=O)(=O)Nc3nc(cc(-c4c(C)cccc4C)n3)OC[C@H]2CC(C)(C)C)n1] | 1.88 | 727.352 | 728.6 | A |
| 1481 | 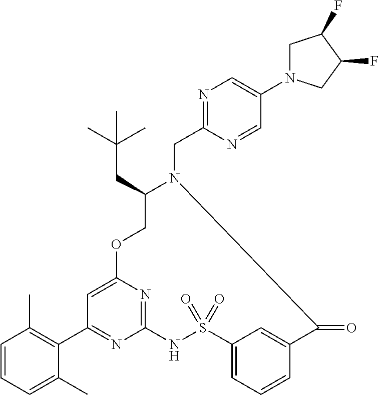 [SMILES: Cc1cccc(C)c1-c1cc2nc(n1)NS(=O)(=O)c1cccc(c1)C(=O)N(Cc1ncc(N3C[C@@H](F)[C@@H](F)C3)cn1)[C@H](CC(C)(C)C)CO2] | 1.45 | 691.275 | 692.4 | A |
| 1482 | 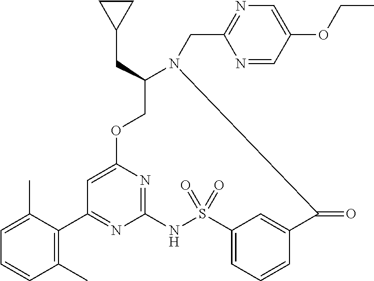 [SMILES: CCOc1cnc(CN2C(=O)c3cccc(c3)S(=O)(=O)Nc3nc(cc(-c4c(C)cccc4C)n3)OC[C@H]2CC2CC2)nc1] | 1.45 | 614.231 | 615.331 | A |
| 1483 | 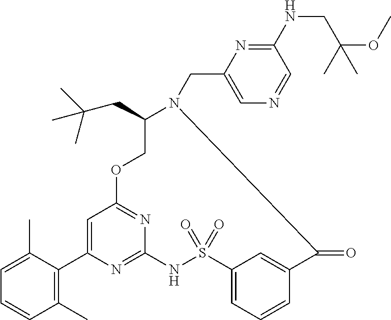 [SMILES: COC(C)(C)CNc1cncc(CN2C(=O)c3cccc(c3)S(=O)(=O)Nc3nc(cc(-c4c(C)cccc4C)n3)OC[C@H]2CC(C)(C)C)n1] | 1.71 | 687.32 | 688.8 | A |
| 1484 | 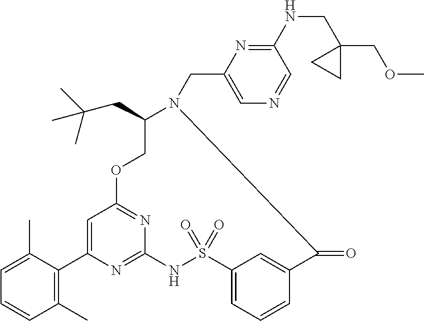 [SMILES: COCC1(CNc2cncc(CN3C(=O)c4cccc(c4)S(=O)(=O)Nc4nc(cc(-c5c(C)cccc5C)n4)OC[C@H]3CC(C)(C)C)n2)CC1] | 1.71 | 699.32 | 700.8 | A |
| 1485 | 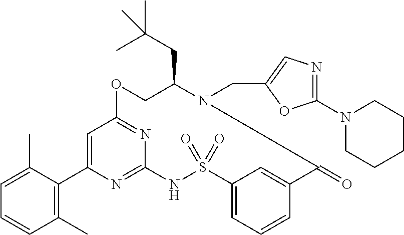 [SMILES: Cc1cccc(C)c1-c1cc2nc(n1)NS(=O)(=O)c1cccc(c1)C(=O)N(Cc1cnc(N3CCCCC3)o1)[C@H](CC(C)(C)C)CO2] | 3.64 | 658.294 | 659.3 | Y |
| 1296 | 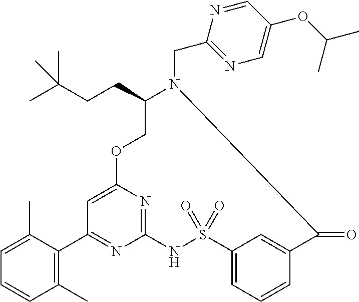 [SMILES: Cc1cccc(C)c1-c1cc2nc(n1)NS(=O)(=O)c1cccc(c1)C(=O)N(Cc1ncc(OC(C)C)cn1)[C@H](CCC(C)(C)C)CO2] | 1.95 | 658.294 | 659.27 | A |
| 1486 | 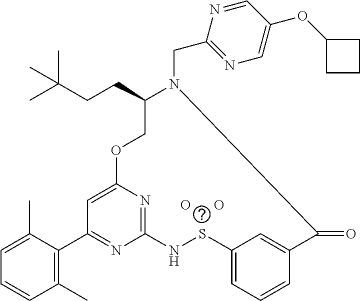 | 2.04 | 670.294 | 671.78 | A |
| 1487 | 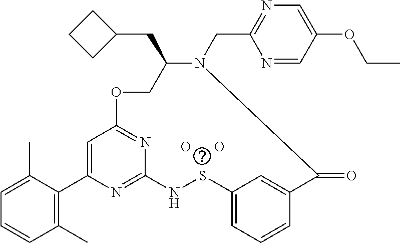 | 1.77 | 628.247 | 629.7 | A |
| 1488 | 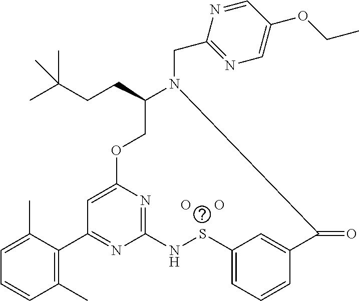 | 1.91 | 644.278 | 645.25 | A |
| 1489 | 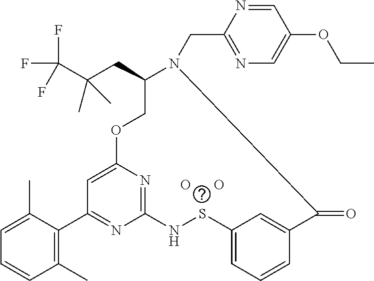 | 1.77 | 684.234 | 685.92 | A |
| 1490 | 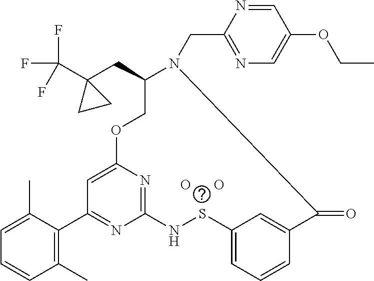 | 1.71 | 682.219 | 683.72 | A |
| 1491 | 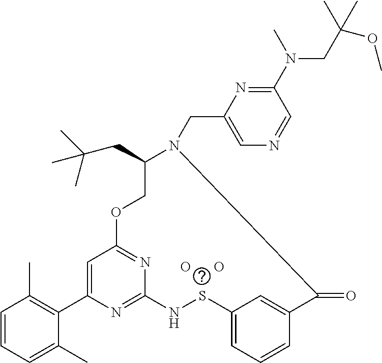 | 1.76 | 701.336 | 702.8 | A |
| 1492 | 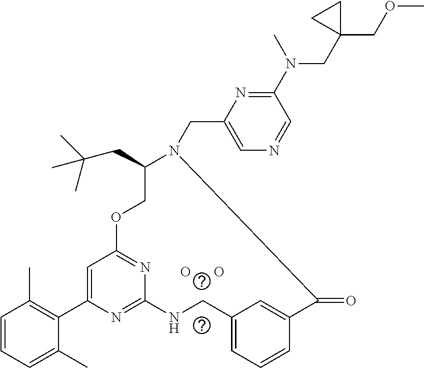 | 1.76 | 713.336 | 714.8 | A |
| 1493 | 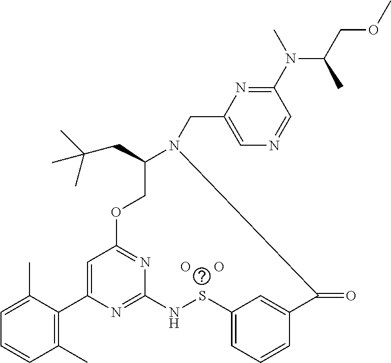 | 1.68 | 687.32 | 688.8 | A |
| 1494 | 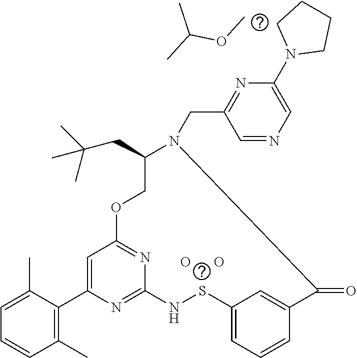 | 1.89 | 727.352 | 728.7 | A |
| 1495 | 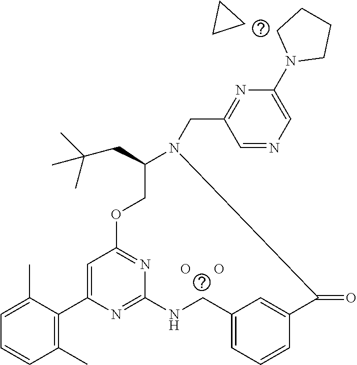 | 1.86 | 695.325 | 696.8 | A |
| 1496 | 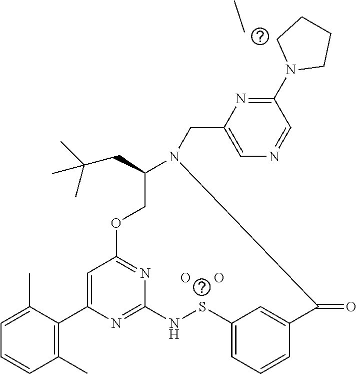 | 1.83 | 683.325 | 684.7 | A |
| 1497 | 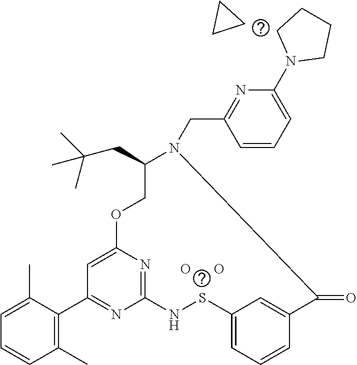 | 1.52 | 694.33 | 695.8 | A |
| 1498 | 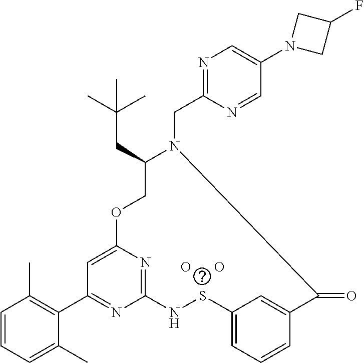 | 1.55 | 659.269 | 660.5 | A |
| 1499 | 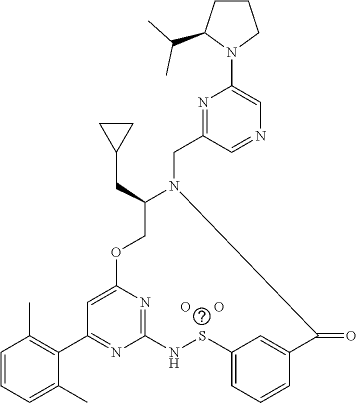 | 1.68 | 681.31 | 682.5 | A |
| 1500 | 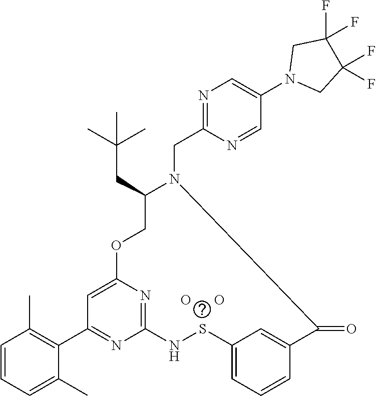 | 1.58 | 727.256 | 728.3 | A |
| 1501 | 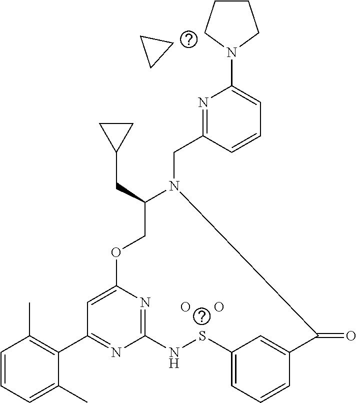 | 1.31 | 678.299 | 679.5 | A |
| 1502 | 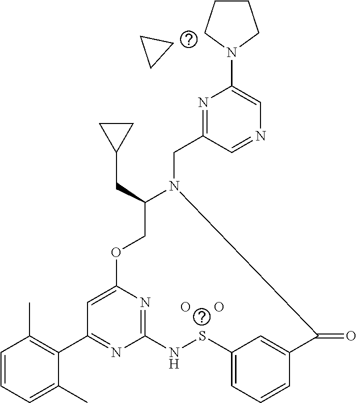 | 1.62 | 679.294 | 680.5 | A |
| 1503 | 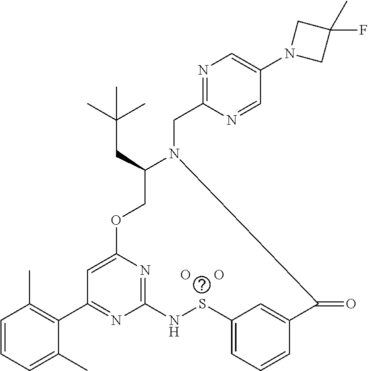 | 1.63 | 673.285 | 674.4 | A |
| 1504 | 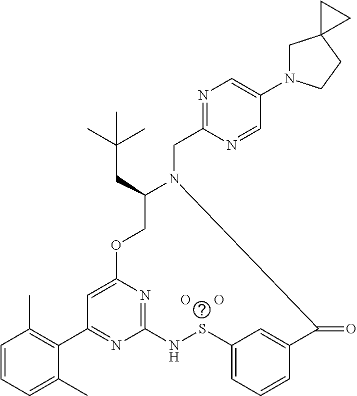 | 1.74 | 681.31 | 682.5 | A |
| 1505 | 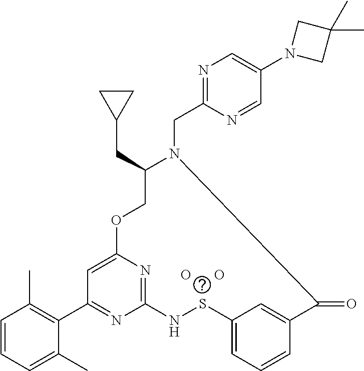 | 1.65 | 653.278 | 654.4 | A |
| 1506 | 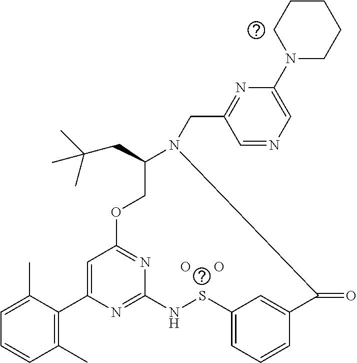 | 1.84 | 683.325 | 684.7 | A |
| 1507 | 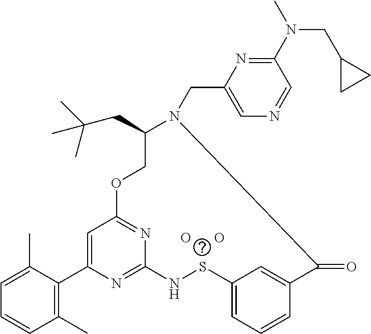 | 1.79 | 669.31 | 670.7 | A |
| 1508 | 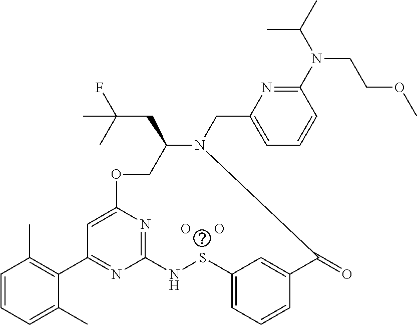 | 3.502 | 704.316 | 705.3 | Y |
| 1509 | 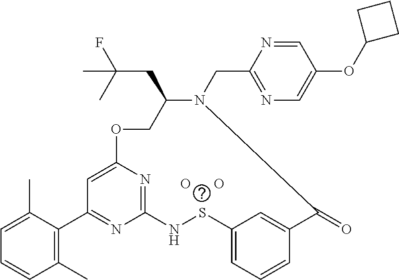 | 4.15 | 660.253 | 661.3 | Y |
| 1510 | 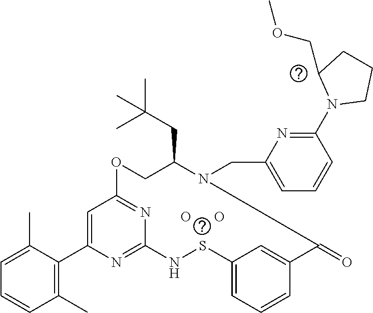 | 3.53 | 712.341 | 713.4 | 1D |
| 1511 | 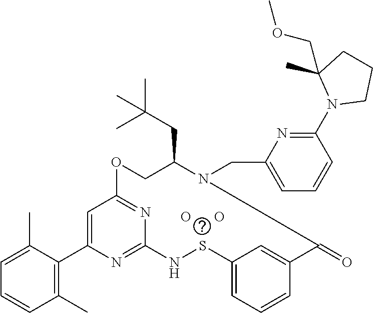 | 3.37 | 712.341 | 713.3 | Y |
| 1512 | 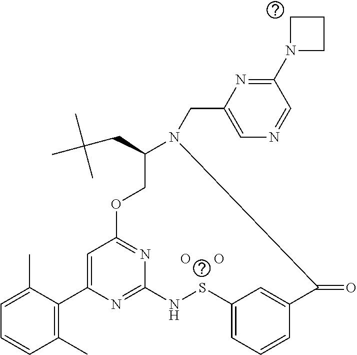 | 1.77 | 655.294 | 656.7 | A |
| 1513 | 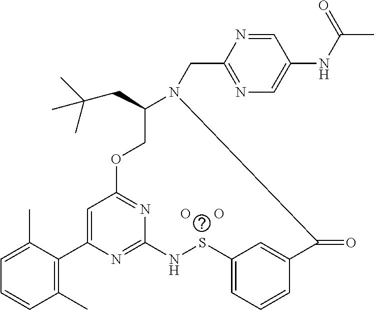 | 1.55 | 643.258 | 644.6 | A(1- 50% gradient) |
| 1514 | 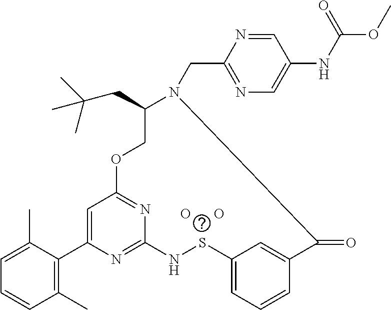 | 1.64 | 659.253 | 660.6 | A |
| 1515 | 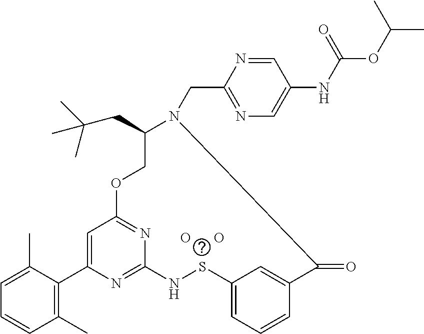 | 1.79 | 687.284 | 688.6 | A |
| 1516 | 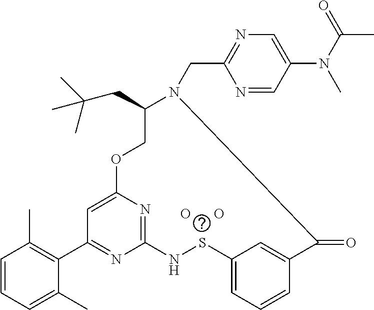 | 1.57 | 657.273 | 658.7 | A |
| 1517 | 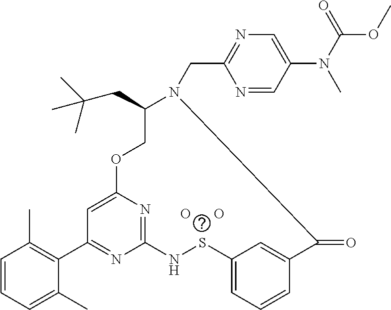 | 1.69 | 673.268 | 674.6 | A |
| 1518 | 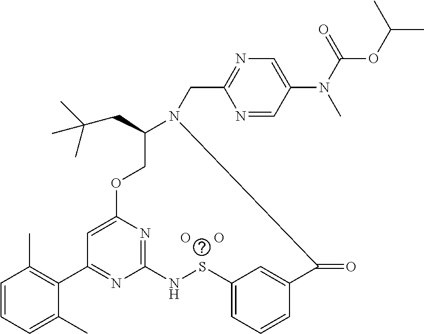 | 1.84 | 701.3 | 702.7 | A |
| 1519 | 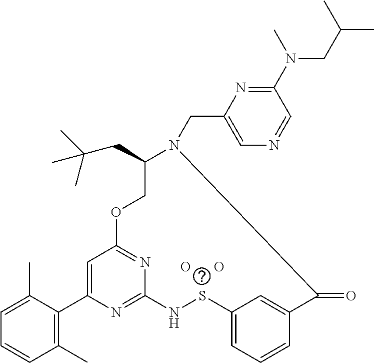 | 1.85 | 671.325 | 672.7 | A |
| 1520 | 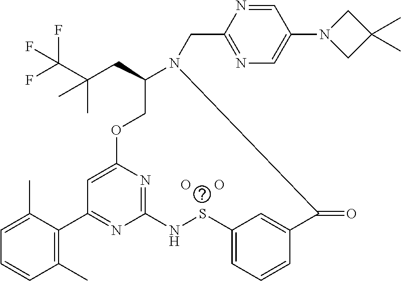 | 1.91 | 723.281 | 724.93 | A |
| 1521 | 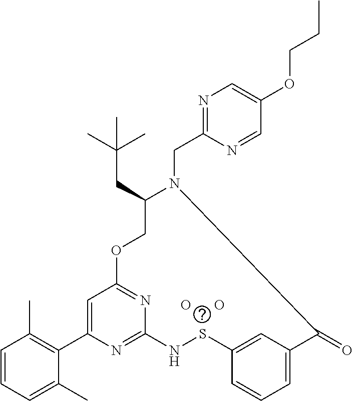 | 1.89 | 644.278 | 645.5 | A |
| 1522 | 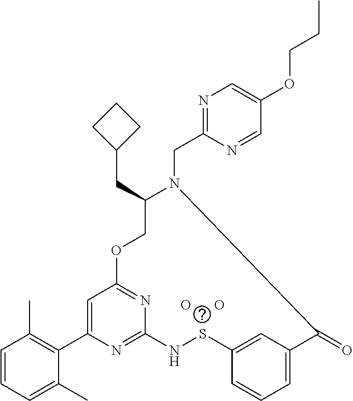 | 1.88 | 642.262 | 643.5 | A |
| 1523 | 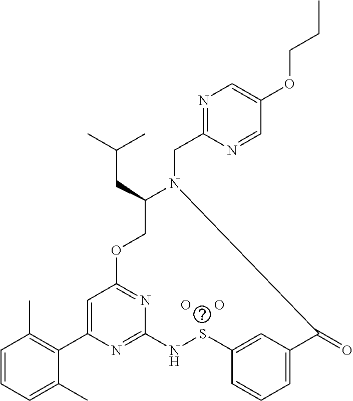 | 1.85 | 630.262 | 631.4 | A |
| 1524 | 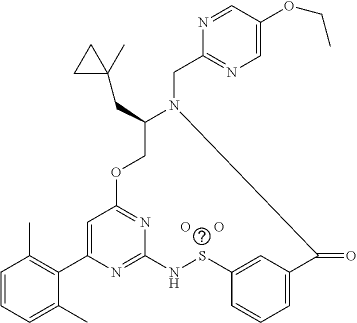 | 1.73 | 628.247 | 629.85 | A |
| 1306 | 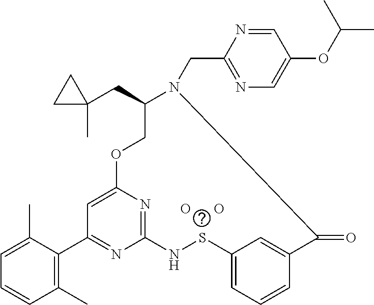 | 1.8 | 642.262 | 643.66 | A |
| 1307 | 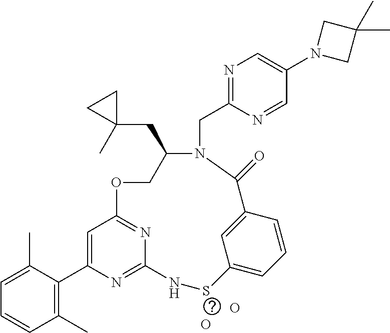 | 1.88 | 667.294 | 668.2 | A |
| 1308 | 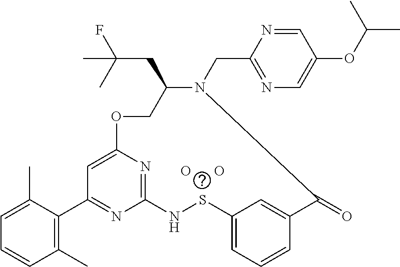 | 4.04 | 648.253 | 649.3 | Y |
| 1525 | 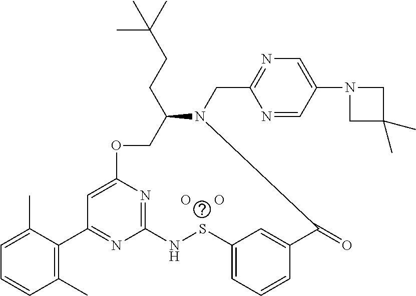 | 2.03 | 683.325 | 684.5 | A |
| 1309 | 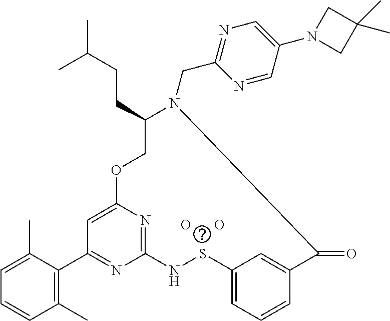 | 1.91 | 669.31 | 670.4 | A |
| 1526 | 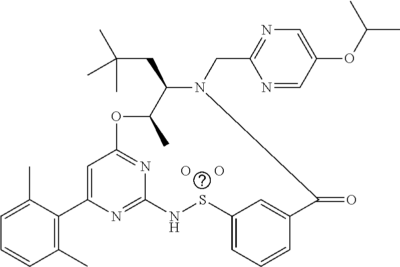 | 1.95 | 658.294 | 659.2 | A |
| 1527 | 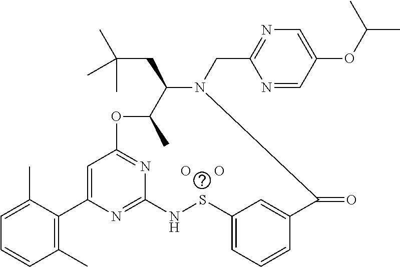 | 1.94 | 658.294 | 659.2 | A |
| 1528 | 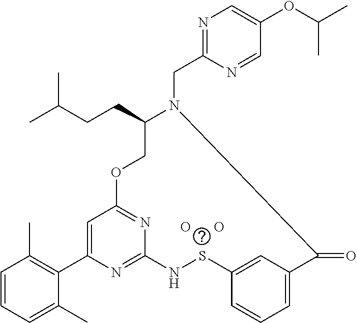 | 2 | 658.294 | 659.67 | A |
| 1529 | 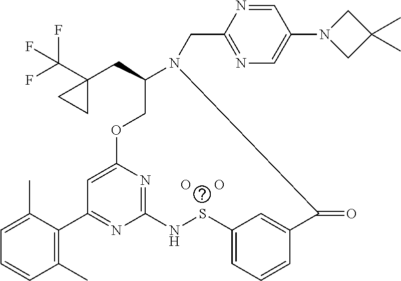 | 1.85 | 721.266 | 722.73 | A |
| 1530 | 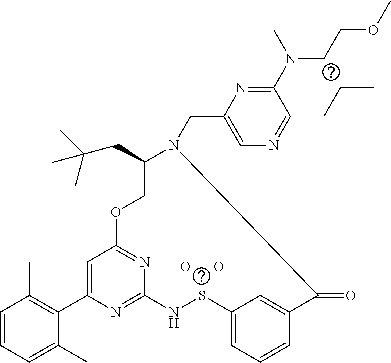 | 1.8 | 715.352 | 716.8 | A |
| 1531 | 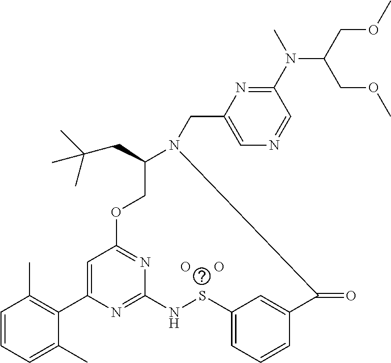 | 1.67 | 717.331 | 718.8 | A |
| 1532 | 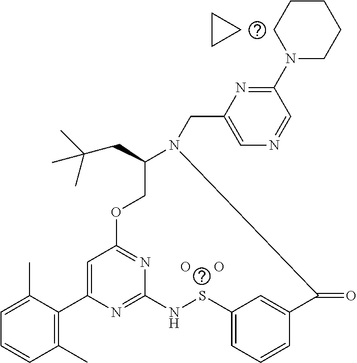 | 1.9 | 709.341 | 710.9 | A |
| 1533 | 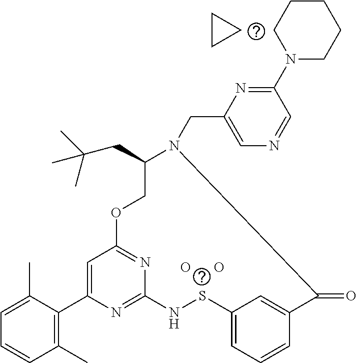 | 1.92 | 709.341 | 710.7 | A |
| 1534 | 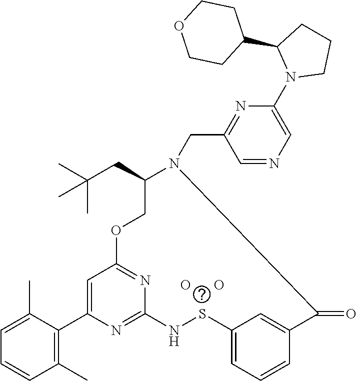 | 1.76 | 739.352 | 740.8 | A |
| 1535 | 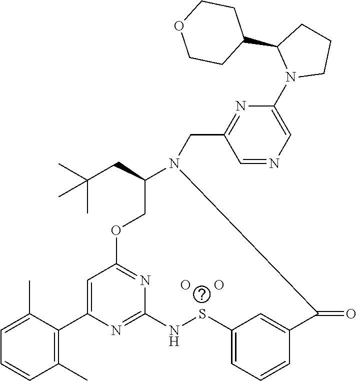 | 1.74 | 739.352 | 740.7 | A |
| 1536 | 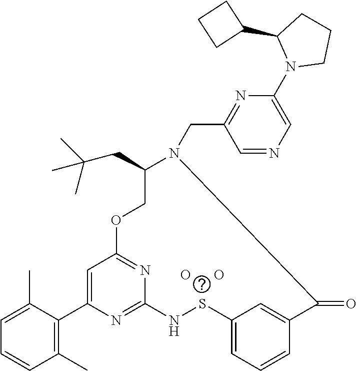 | 1.97 | 709.341 | 710.8 | A |
| 1537 | 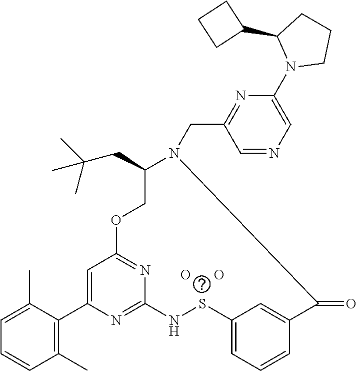 | 1.97 | 709.341 | 710.8 | A |
| 1538 | 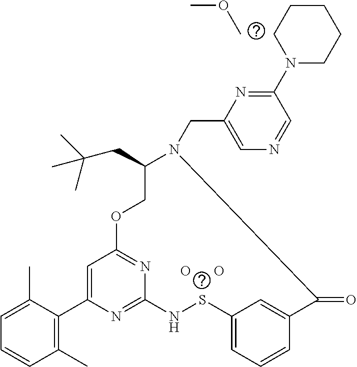 | 1.79 | 713.336 | 715 | A |
| 1539 | 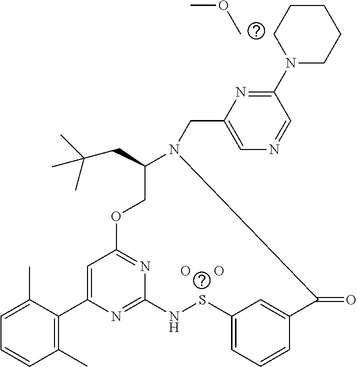 | 1.78 | 713.336 | 715 | A |
| 1540 | 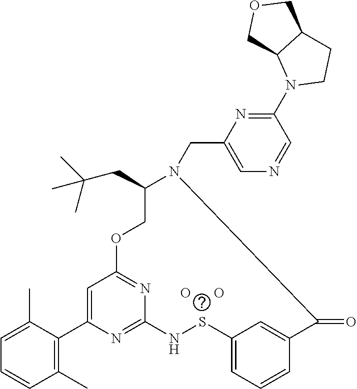 | 1.69 | 697.305 | 698.9 | A |
| 1541 | 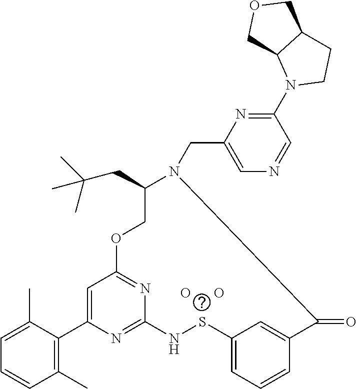 | 1.69 | 697.305 | 698.8 | A |
| 1542 | 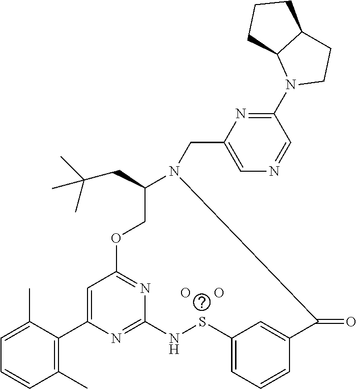 | 1.87 | 695.325 | 696.9 | A |
| 1543 | 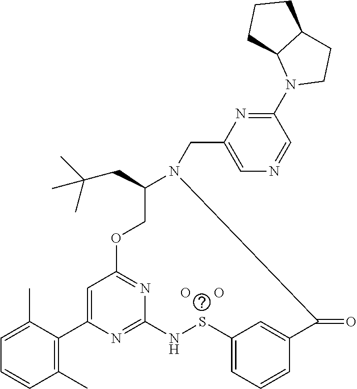 | 1.87 | 695.325 | 696.9 | A |
| 1544 | 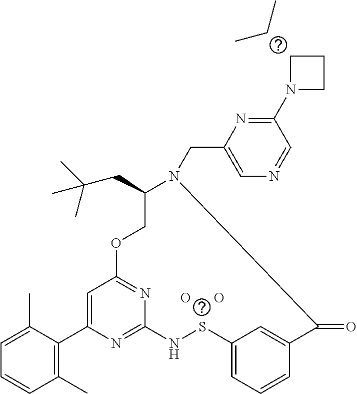 | 1.93 | 683.325 | 684.9 | A |
| 1545 | 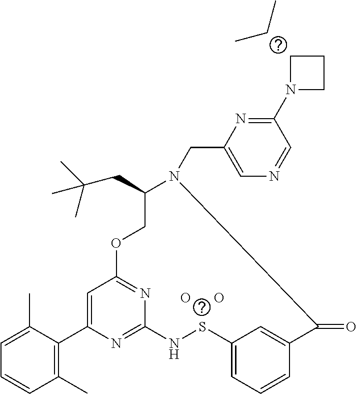 | 1.94 | 683.325 | 684.9 | A |
| 1546 | 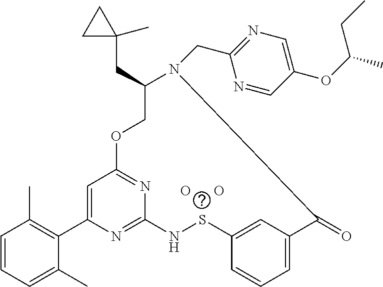 | 1.74 | 656.278 | 657.4 | A |
| 1547 | 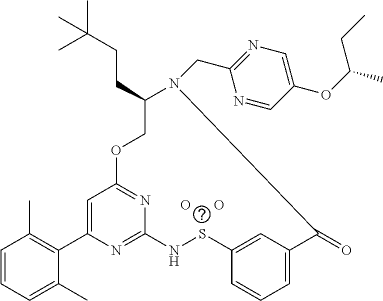 | 1.87 | 672.309 | 673.2 | A |
| 1548 | 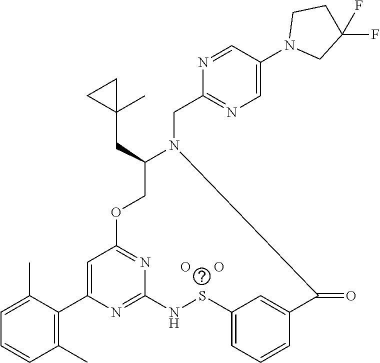 | 1.71 | 689.26 | 690.3 | A |
| 1549 | 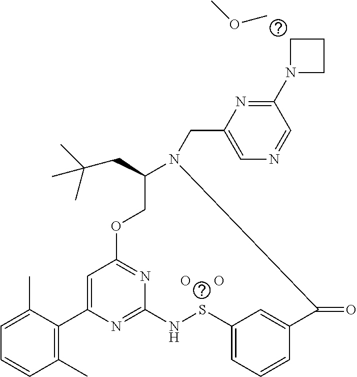 | 1.73 | 685.305 | 686.8 | A |
| 1550 | 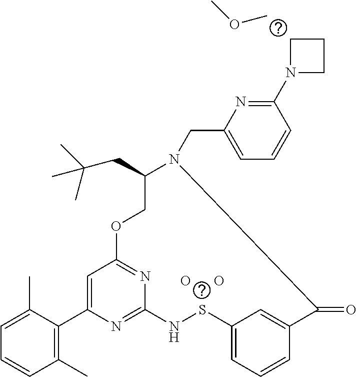 | 1.41 | 684.309 | 685.8 | A |
| 1551 | 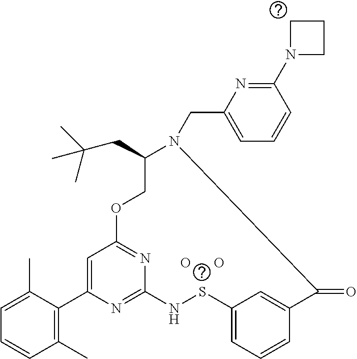 | 1.41 | 654.299 | 655.8 | A |
| 1552 | 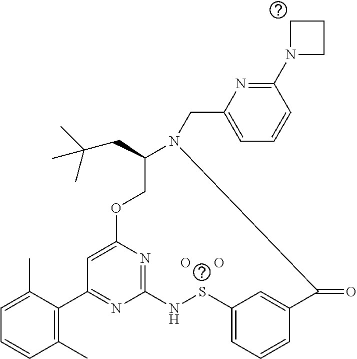 | 1.32 | 655.294 | 656.8 | A |
| 1553 | 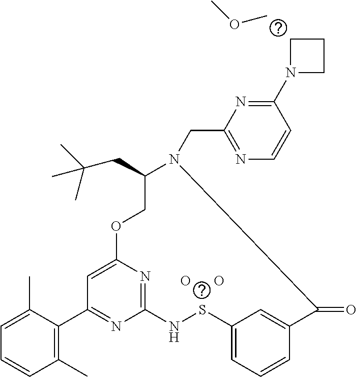 | 1.32 | 685.305 | 686.8 | A |
| 1554 | 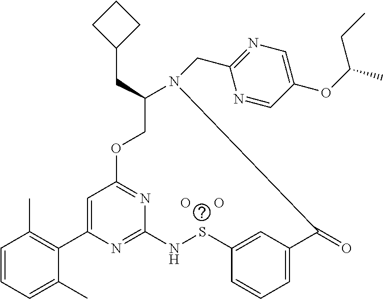 | 1.7 | 656.278 | 657.174 | A |
| 1555 | 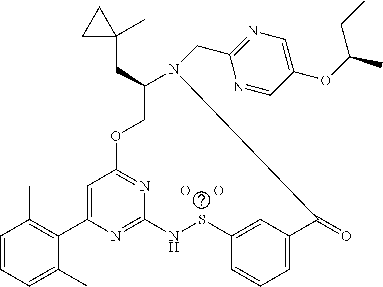 | 1.7 | 656.278 | 657.2 | A |
| 1556 | 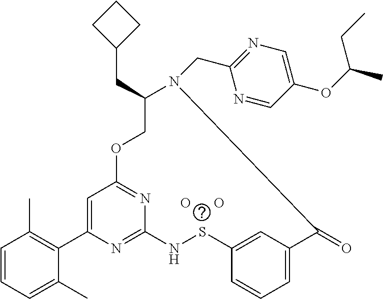 | 1.74 | 656.278 | 657.2 | A |
| 1557 | 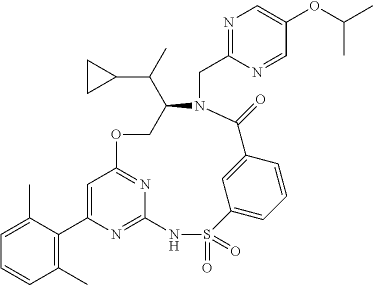 [SMILES: Cc1cccc(C)c1-c1cc2nc(n1)NS(=O)(=O)c1cccc(c1)C(=O)N(Cc1ncc(OC(C)C)cn1)[C@H](C(C)C1CC1)CO2] | 1.82 | 642.262 | 643.2 | A |
| 1558 | 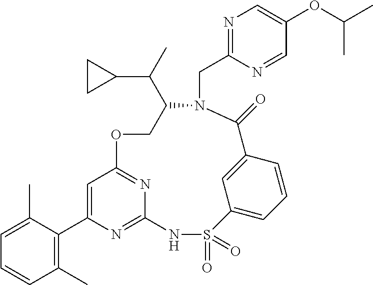 | 1.82 | 642.262 | 643.2 | A |
| 1559 | 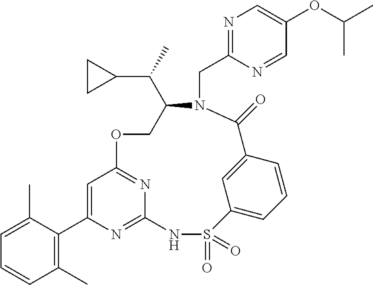 [SMILES: Cc1cccc(C)c1-c1cc2nc(n1)NS(=O)(=O)c1cccc(c1)C(=O)N(Cc1ncc(OC(C)C)cn1)[C@H]([C@@H](C)C1CC1)CO2] | 1.82 | 642.262 | 643.2 | A |
| 1560 | 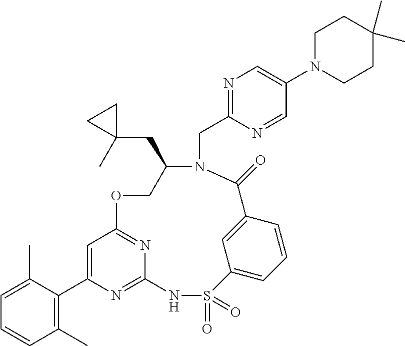 [SMILES: Cc1cccc(C)c1-c1cc2nc(n1)NS(=O)(=O)c1cccc(c1)C(=O)N(Cc1ncc(N3CCC(C)(C)CC3)cn1)[C@H](CC1(C)CC1)CO2] | 2 | 695.325 | 696 | A |
| 1561 | 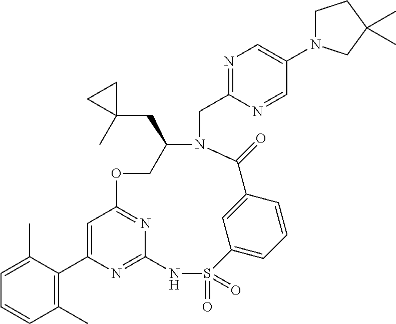 [SMILES: Cc1cccc(C)c1-c1cc2nc(n1)NS(=O)(=O)c1cccc(c1)C(=O)N(Cc1ncc(N3CCC(C)(C)C3)cn1)[C@H](CC1(C)CC1)CO2] | 1.91 | 681.31 | 682.2 | A |
| 1562 | 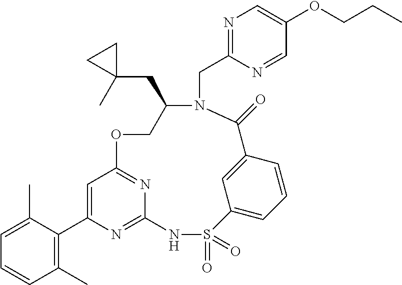 [SMILES: CCCOc1cnc(CN2C(=O)c3cccc(c3)S(=O)(=O)Nc3nc(cc(-c4c(C)cccc4C)n3)OC[C@H]2CC2(C)CC2)nc1] | 1.85 | 642.262 | 643.2 | A |
| 1563 | 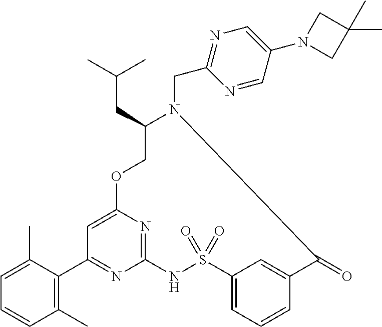 [SMILES: Cc1cccc(C)c1-c1cc2nc(n1)NS(=O)(=O)c1cccc(c1)C(=O)N(Cc1ncc(N3CC(C)(C)C3)cn1)[C@H](CC(C)C)CO2] | 1.87 | 655.294 | 656.5 | A |
| 1564 | 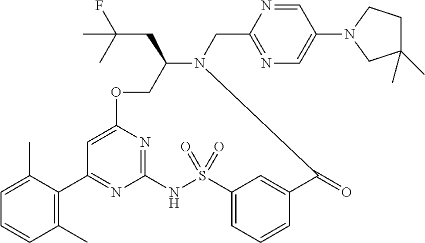 [SMILES: Cc1cccc(C)c1-c1cc2nc(n1)NS(=O)(=O)c1cccc(c1)C(=O)N(Cc1ncc(N3CCC(C)(C)C3)cn1)[C@H](CC(C)(C)F)CO2] | 4.504 | 687.3 | 688.4 | Y |
| 1565 | 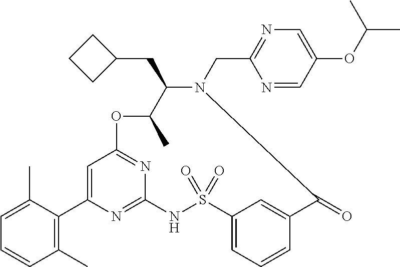 [SMILES: Cc1cccc(C)c1-c1cc2nc(n1)NS(=O)(=O)c1cccc(c1)C(=O)N(Cc1ncc(OC(C)C)cn1)[C@H](CC1CCC1)[C@@H](C)O2] | 1.94 | 656.278 | 657.2 | A |
| 1566 | 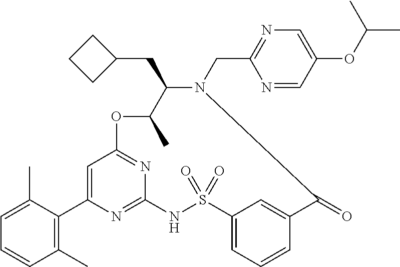 [SMILES: Cc1cccc(C)c1-c1cc2nc(n1)NS(=O)(=O)c1cccc(c1)C(=O)N(Cc1ncc(OC(C)C)cn1)[C@H](CC1CCC1)[C@@H](C)O2] | 1.94 | 656.278 | 657.2 | A |
| 1567 | 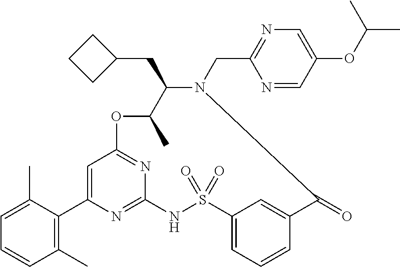 [SMILES: Cc1cccc(C)c1-c1cc2nc(n1)NS(=O)(=O)c1cccc(c1)C(=O)N(Cc1ncc(OC(C)C)cn1)[C@H](CC1CCC1)[C@@H](C)O2] | 1.92 | 656.278 | 657.2 | A |
| 1568 | 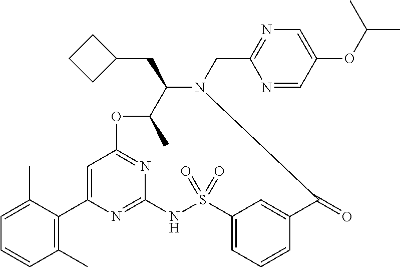 [SMILES: Cc1cccc(C)c1-c1cc2nc(n1)NS(=O)(=O)c1cccc(c1)C(=O)N(Cc1ncc(OC(C)C)cn1)[C@H](CC1CCC1)[C@@H](C)O2] | 1.92 | 656.278 | 657.2 | A |
| 1569 | 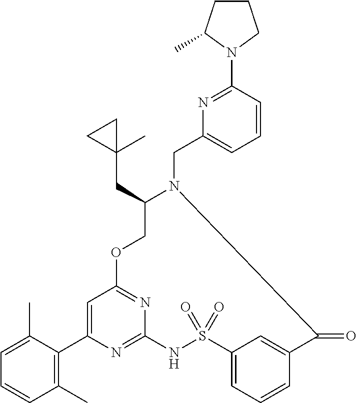 [SMILES: Cc1cccc(C)c1-c1cc2nc(n1)NS(=O)(=O)c1cccc(c1)C(=O)N(Cc1cccc(N3CCC[C@H]3C)n1)[C@H](CC1(C)CC1)CO2] | 1.45 | 666.299 | 667.5 | A |
| 1570 | 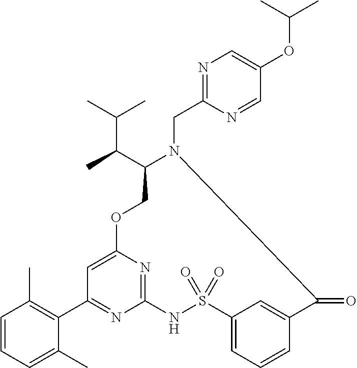 [SMILES: Cc1cccc(C)c1-c1cc2nc(n1)NS(=O)(=O)c1cccc(c1)C(=O)N(Cc1ncc(OC(C)C)cn1)[C@H]([C@@H](C)C(C)C)CO2] | 1.86 | 644.278 | 645.9 | A |
| 1571 | 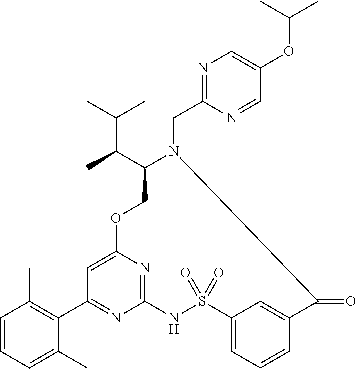 [SMILES: Cc1cccc(C)c1-c1cc2nc(n1)NS(=O)(=O)c1cccc(c1)C(=O)N(Cc1ncc(OC(C)C)cn1)[C@H]([C@@H](C)C(C)C)CO2] | 1.88 | 644.278 | 645.8 | A |
| 1572 | 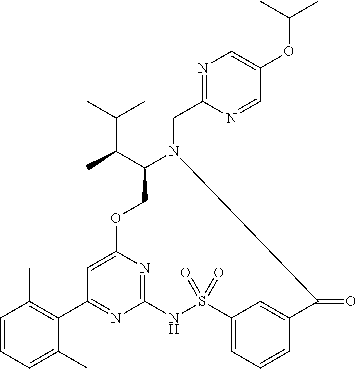 [SMILES: Cc1cccc(C)c1-c1cc2nc(n1)NS(=O)(=O)c1cccc(c1)C(=O)N(Cc1ncc(OC(C)C)cn1)[C@H]([C@@H](C)C(C)C)CO2] | 1.88 | 644.278 | 645.8 | A |
| 1573 | 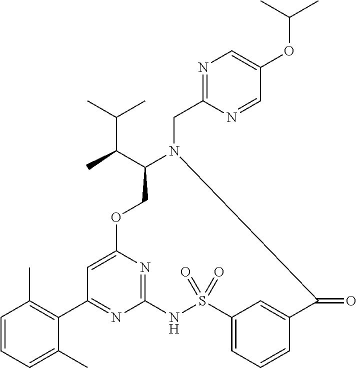 [SMILES: Cc1cccc(C)c1-c1cc2nc(n1)NS(=O)(=O)c1cccc(c1)C(=O)N(Cc1ncc(OC(C)C)cn1)[C@H]([C@@H](C)C(C)C)CO2] | 1.86 | 644.278 | 645.8 | A |
| 1574 | 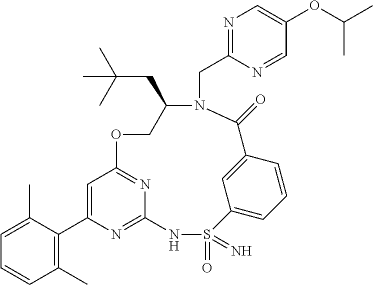 [SMILES: Cc1cccc(C)c1-c1cc2nc(n1)NS(=N)(=O)c1cccc(c1)C(=O)N(Cc1ncc(OC(C)C)cn1)[C@H](CC(C)(C)C)CO2] | 1.59 | 643.294 | 644.2 | A |
| 1575 | 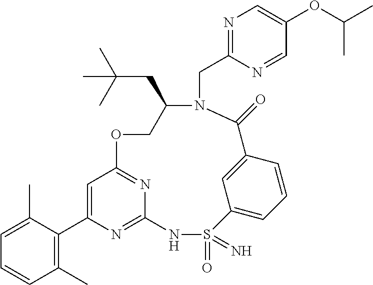 [SMILES: Cc1cccc(C)c1-c1cc2nc(n1)NS(=N)(=O)c1cccc(c1)C(=O)N(Cc1ncc(OC(C)C)cn1)[C@H](CC(C)(C)C)CO2] | 1.53 | 643.294 | 644.2 | A |
| 1576 | 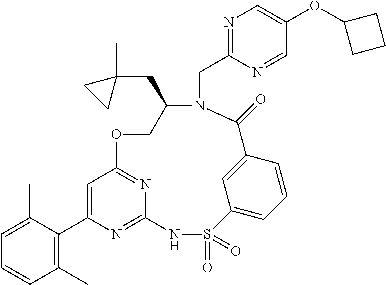 [SMILES: Cc1cccc(C)c1-c1cc2nc(n1)NS(=O)(=O)c1cccc(c1)C(=O)N(Cc1ncc(OC3CCC3)cn1)[C@H](CC1(C)CC1)CO2] | 1.89 | 654.262 | 655.2 | A |
| 1577 | 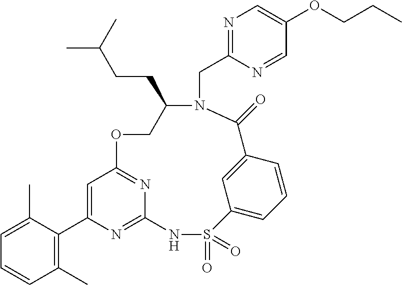 [SMILES: CCCOc1cnc(CN2C(=O)c3cccc(c3)S(=O)(=O)Nc3nc(cc(-c4c(C)cccc4C)n3)OC[C@H]2CCC(C)C)nc1] | 1.96 | 644.278 | 645.2 | A |
| 1310 | 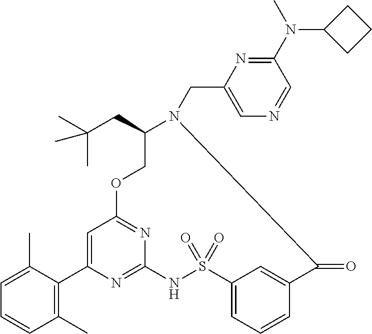 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)C)CO2] | 4.64 | 669.31 | 670.2 | Y |
| 1578 | 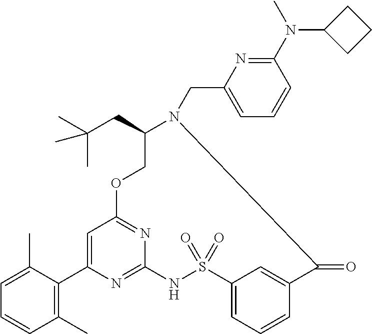 [SMILES: Cc1cccc(C)c1-c1cc2nc(n1)NS(=O)(=O)c1cccc(c1)C(=O)N(Cc1cccc(N(C)C3CCC3)n1)[C@H](CC(C)(C)C)CO2] | 1.48 | 668.314 | 669.8 | A |
| 1579 | 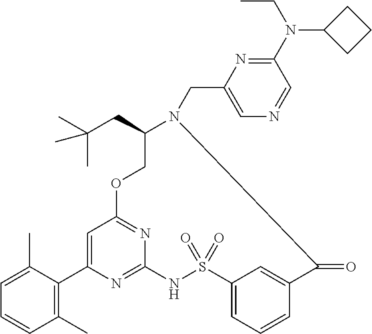 [SMILES: CCN(c1cncc(CN2C(=O)c3cccc(c3)S(=O)(=O)Nc3nc(cc(-c4c(C)cccc4C)n3)OC[C@H]2CC(C)(C)C)n1)C1CCC1] | 1.88 | 683.325 | 684.8 | A |
| 1580 | 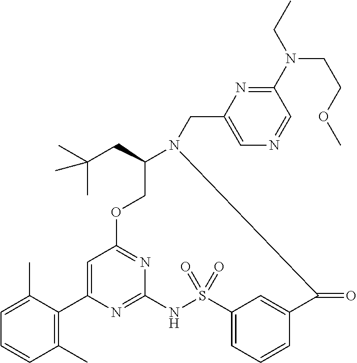 [SMILES: CCN(CCOC)c1cncc(CN2C(=O)c3cccc(c3)S(=O)(=O)Nc3nc(cc(-c4c(C)cccc4C)n3)OC[C@H]2CC(C)(C)C)n1] | 1.7 | 687.32 | 688.9 | A |
| 1581 | 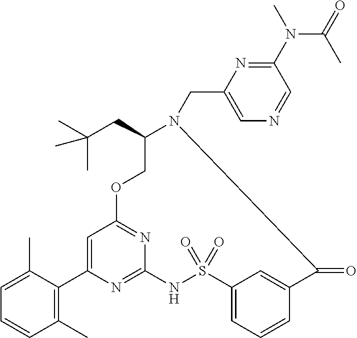 [SMILES: CC(=O)N(C)c1cncc(CN2C(=O)c3cccc(c3)S(=O)(=O)Nc3nc(cc(-c4c(C)cccc4C)n3)OC[C@H]2CC(C)(C)C)n1] | 1.59 | 657.273 | 658.8 | A |
| 1582 | 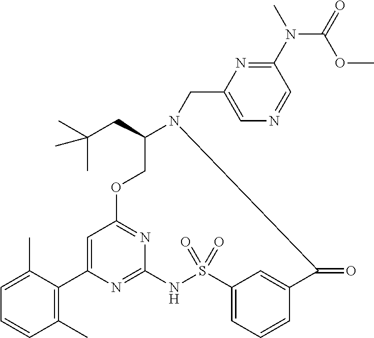 [SMILES: COC(=O)N(C)c1cncc(CN2C(=O)c3cccc(c3)S(=O)(=O)Nc3nc(cc(-c4c(C)cccc4C)n3)OC[C@H]2CC(C)(C)C)n1] | 1.72 | 673.268 | 674.2 | A |
| 1583 | 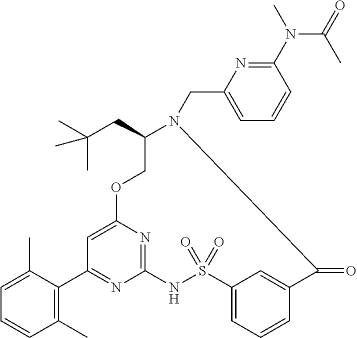 [SMILES: CC(=O)N(C)c1cccc(CN2C(=O)c3cccc(c3)S(=O)(=O)Nc3nc(cc(-c4c(C)cccc4C)n3)OC[C@H]2CC(C)(C)C)n1] | 1.66 | 656.278 | 657.9 | A |
| 1584 | 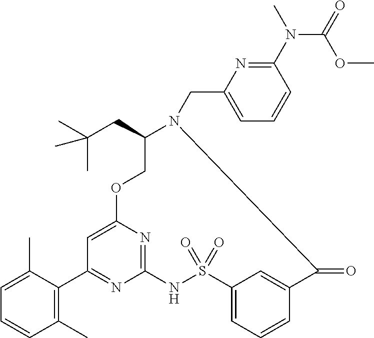 [SMILES: COC(=O)N(C)c1cccc(CN2C(=O)c3cccc(c3)S(=O)(=O)Nc3nc(cc(-c4c(C)cccc4C)n3)OC[C@H]2CC(C)(C)C)n1] | 1.83 | 672.273 | 673.8 | A |
| 1342 | 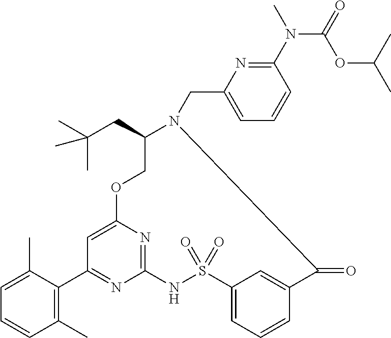 [SMILES: Cc1cccc(C)c1-c1cc2nc(n1)NS(=O)(=O)c1cccc(c1)C(=O)N(Cc1cccc(N(C)C(=O)OC(C)C)n1)[C@H](CC(C)(C)C)CO2] | 2.01 | 700.304 | 701.8 | A |
| 1311 | 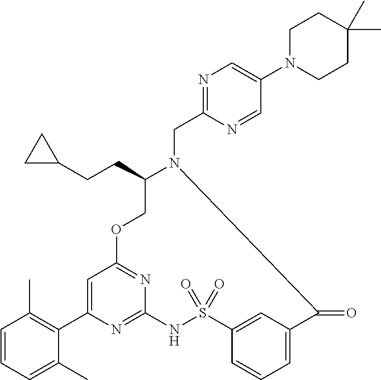 [SMILES: Cc1cccc(C)c1-c1cc2nc(n1)NS(=O)(=O)c1cccc(c1)C(=O)N(Cc1ncc(N3CCC(C)(C)CC3)cn1)[C@H](CCC1CC1)CO2] | 1.83 | 695.325 | 696.5 | A |
| 1585 | 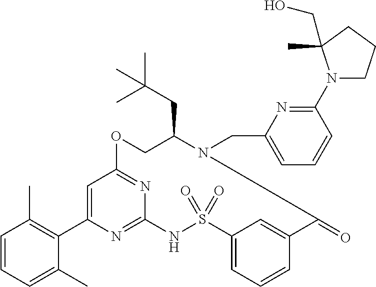 [SMILES: Cc1cccc(C)c1-c1cc2nc(n1)NS(=O)(=O)c1cccc(c1)C(=O)N(Cc1cccc(N3CCC[C@@]3(C)CO)n1)[C@H](CC(C)(C)C)CO2] | 3.11 | 698.325 | 699.4 | 1D |
| 1586 | 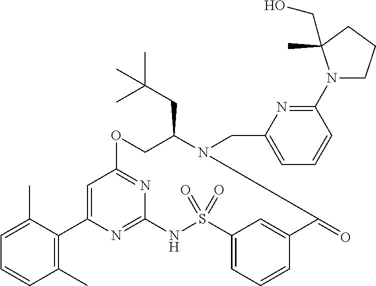 [SMILES: Cc1cccc(C)c1-c1cc2nc(n1)NS(=O)(=O)c1cccc(c1)C(=O)N(Cc1cccc(N3CCC[C@@]3(C)CO)n1)[C@H](CC(C)(C)C)CO2] | 3.11 | 698.325 | 699.4 | 1D |
| 1587 | 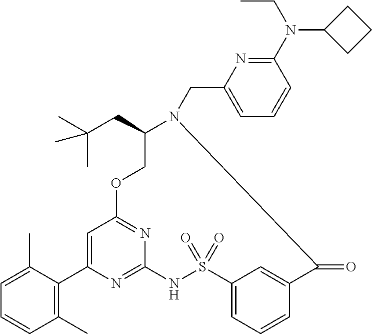 [SMILES: CCN(c1cccc(CN2C(=O)c3cccc(c3)S(=O)(=O)Nc3nc(cc(-c4c(C)cccc4C)n3)OC[C@H]2CC(C)(C)C)n1)C1CCC1] | 1.53 | 682.33 | 683.9 | A |
| 1588 | 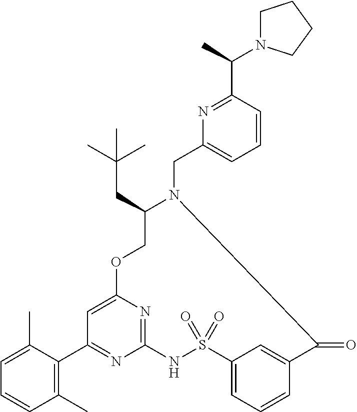 [SMILES: Cc1cccc(C)c1-c1cc2nc(n1)NS(=O)(=O)c1cccc(c1)C(=O)N(Cc1cccc([C@@H](C)N3CCCC3)n1)[C@H](CC(C)(C)C)CO2] | 1.46 | 682.33 | 683.5 | A |
| 1589 | 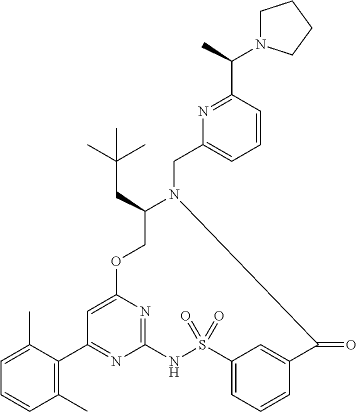 [SMILES: Cc1cccc(C)c1-c1cc2nc(n1)NS(=O)(=O)c1cccc(c1)C(=O)N(Cc1cccc([C@@H](C)N3CCCC3)n1)[C@H](CC(C)(C)C)CO2] | 1.46 | 682.33 | 683.5 | A |
| 1590 | 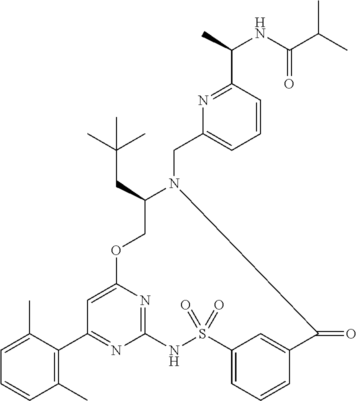 [SMILES: Cc1cccc(C)c1-c1cc2nc(n1)NS(=O)(=O)c1cccc(c1)C(=O)N(Cc1cccc([C@@H](C)NC(=O)C(C)C)n1)[C@H](CC(C)(C)C)CO2] | 1.74 | 698.325 | 699.5 | A |
| 1591 | 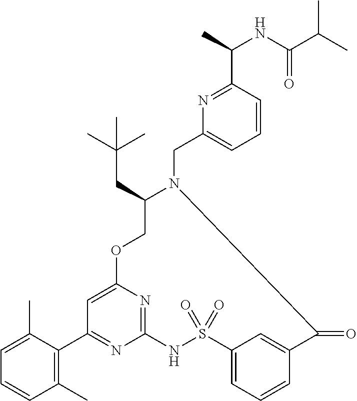 [SMILES: Cc1cccc(C)c1-c1cc2nc(n1)NS(=O)(=O)c1cccc(c1)C(=O)N(Cc1cccc([C@@H](C)NC(=O)C(C)C)n1)[C@H](CC(C)(C)C)CO2] | 1.72 | 698.325 | 699.5 | A |
| 1592 | 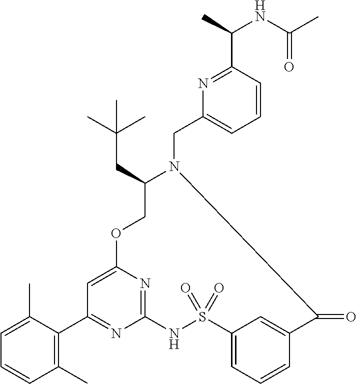 [SMILES: CC(=O)N[C@H](C)c1cccc(CN2C(=O)c3cccc(c3)S(=O)(=O)Nc3nc(cc(-c4c(C)cccc4C)n3)OC[C@H]2CC(C)(C)C)n1] | 1.57 | 670.294 | 671.5 | A |
| 1593 | 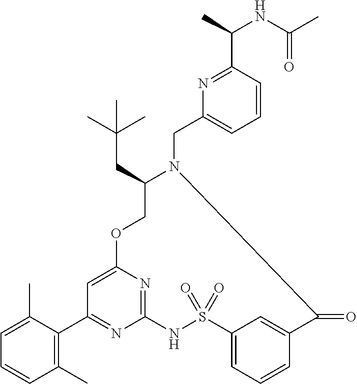 [SMILES: CC(=O)N[C@H](C)c1cccc(CN2C(=O)c3cccc(c3)S(=O)(=O)Nc3nc(cc(-c4c(C)cccc4C)n3)OC[C@H]2CC(C)(C)C)n1] | 1.61 | 670.294 | 671.4 | A |
| 1298 | 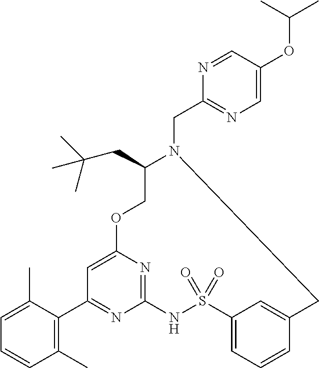 [SMILES: Cc1cccc(C)c1-c1cc2nc(n1)NS(=O)(=O)c1cccc(c1)CN(Cc1ncc(OC(C)C)cn1)[C@H](CC(C)(C)C)CO2] | 2.05 | 630.299 | 631.4 | A |
| 1594 | 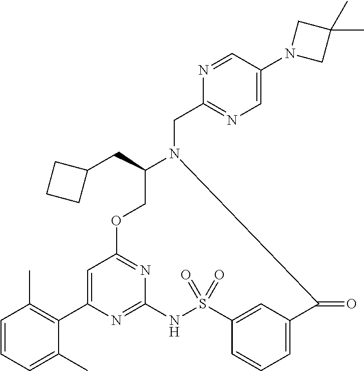 [SMILES: Cc1cccc(C)c1-c1cc2nc(n1)NS(=O)(=O)c1cccc(c1)C(=O)N(Cc1ncc(N3CC(C)(C)C3)cn1)[C@H](CC1CCC1)CO2] | 1.67 | 667.294 | 668.4 | A |
| 1595 | 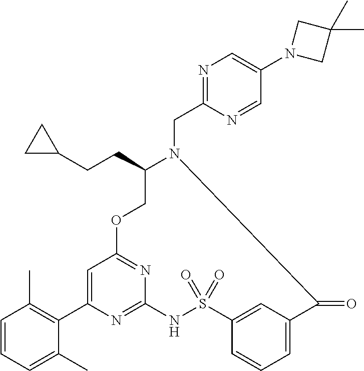 [SMILES: Cc1cccc(C)c1-c1cc2nc(n1)NS(=O)(=O)c1cccc(c1)C(=O)N(Cc1ncc(N3CC(C)(C)C3)cn1)[C@H](CCC1CC1)CO2] | 1.69 | 667.294 | 668.4 | A |
| 1312 | 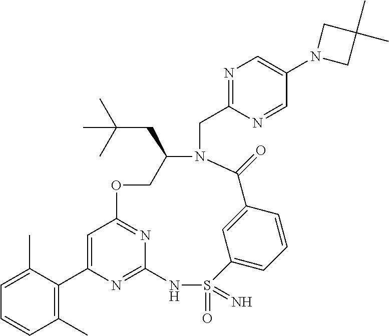 [SMILES: Cc1cccc(C)c1-c1cc2nc(n1)NS(=N)(=O)c1cccc(c1)C(=O)N(Cc1ncc(N3CC(C)(C)C3)cn1)[C@H](CC(C)(C)C)CO2] | 1.62 | 668.326 | 669.3 | A |
| 1313 | 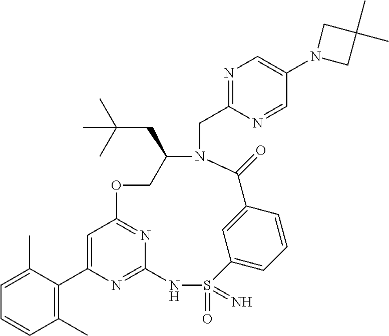 [SMILES: Cc1cccc(C)c1-c1cc2nc(n1)NS(=N)(=O)c1cccc(c1)C(=O)N(Cc1ncc(N3CC(C)(C)C3)cn1)[C@H](CC(C)(C)C)CO2] | 1.61 | 668.326 | 669 | A |
| 1596 | 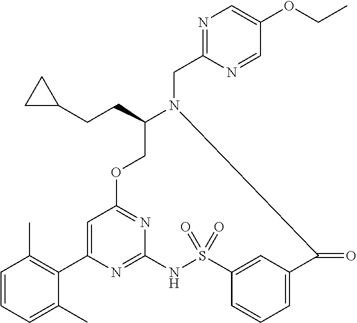 [SMILES: CCOc1cnc(CN2C(=O)c3cccc(c3)S(=O)(=O)Nc3nc(cc(-c4c(C)cccc4C)n3)OC[C@H]2CCC2CC2)nc1] | 1.56 | 628.247 | 629.53 | A |
| 1597 | 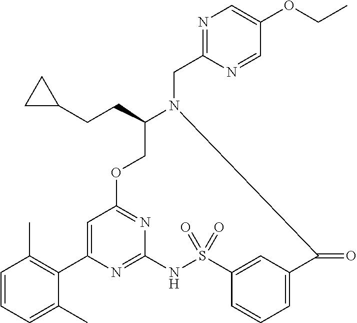 [SMILES: CCOc1cnc(CN2C(=O)c3cccc(c3)S(=O)(=O)Nc3nc(cc(-c4c(C)cccc4C)n3)OC[C@H]2CCC2CC2)nc1] | 1.64 | 642.262 | 643.49 | A |
| 1598 | 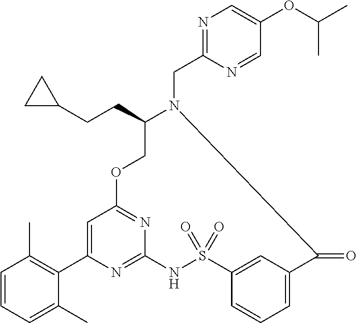 [SMILES: Cc1cccc(C)c1-c1cc2nc(n1)NS(=O)(=O)c1cccc(c1)C(=O)N(Cc1ncc(OC(C)C)cn1)[C@H](CCC1CC1)CO2] | 1.73 | 656.278 | 657.44 | A |
| 1599 | 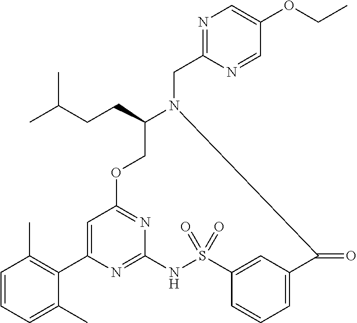 [SMILES: CCOc1cnc(CN2C(=O)c3cccc(c3)S(=O)(=O)Nc3nc(cc(-c4c(C)cccc4C)n3)OC[C@H]2CCC(C)C)nc1] | 1.64 | 630.262 | 631.43 | A |
| 1600 | 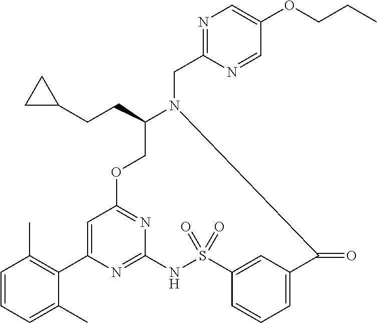 [SMILES: CCCOc1cnc(CN2C(=O)c3cccc(c3)S(=O)(=O)Nc3nc(cc(-c4c(C)cccc4C)n3)OC[C@H]2CCC2CC2)nc1] | 1.67 | 642.262 | 643.5 | A |
| 1601 | 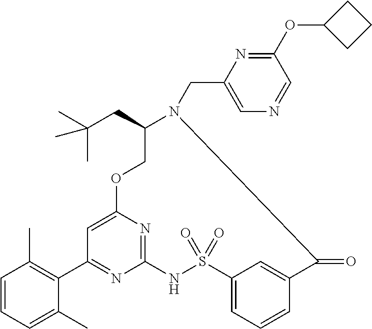 [SMILES: Cc1cccc(C)c1-c1cc2nc(n1)NS(=O)(=O)c1cccc(c1)C(=O)N(Cc1cncc(OC3CCC3)n1)[C@H](CC(C)(C)C)CO2] | 1.92 | 656.278 | 657.8 | A |
| 1602 | 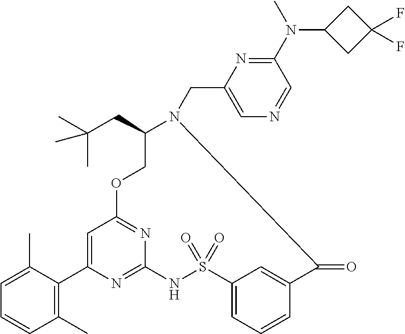 [SMILES: Cc1cccc(C)c1-c1cc2nc(n1)NS(=O)(=O)c1cccc(c1)C(=O)N(Cc1cncc(N(C)C3CC(F)(F)C3)n1)[C@H](CC(C)(C)C)CO2] | 1.82 | 705.291 | 706.8 | A |
| 1603 | 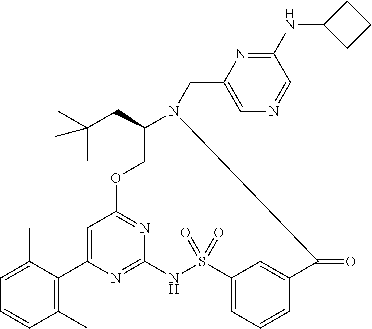 [SMILES: Cc1cccc(C)c1-c1cc2nc(n1)NS(=O)(=O)c1cccc(c1)C(=O)N(Cc1cncc(NC3CCC3)n1)[C@H](CC(C)(C)C)CO2] | 1.75 | 655.294 | 656.8 | D |
| 1604 | 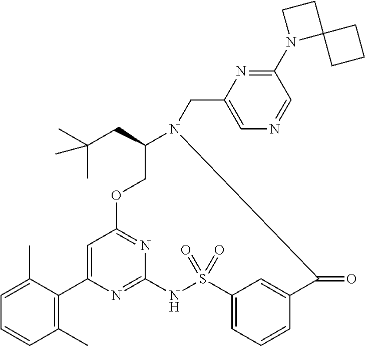 [SMILES: Cc1cccc(C)c1-c1cc2nc(n1)NS(=O)(=O)c1cccc(c1)C(=O)N(Cc1cncc(N3CCC34CCC4)n1)[C@H](CC(C)(C)C)CO2] | 2.15 | 681.31 | 682.4 | A (ammonium formate modifier) |
| 1605 | 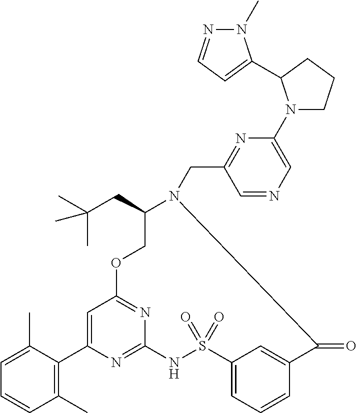 [SMILES: Cc1cccc(C)c1-c1cc2nc(n1)NS(=O)(=O)c1cccc(c1)C(=O)N(Cc1cncc(N3CCCC3c3ccnn3C)n1)[C@H](CC(C)(C)C)CO2] | 1.66 | 735.332 | 736.8 | A |
| 1606 | 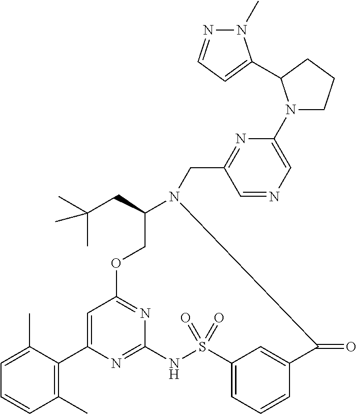 [SMILES: Cc1cccc(C)c1-c1cc2nc(n1)NS(=O)(=O)c1cccc(c1)C(=O)N(Cc1cncc(N3CCCC3c3ccnn3C)n1)[C@H](CC(C)(C)C)CO2] | 1.68 | 735.332 | 736.8 | A |
| 1607 | 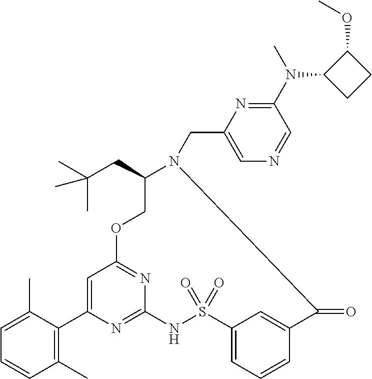 [SMILES: CO[C@@H]1CC[C@@H]1N(C)c1cncc(CN2C(=O)c3cccc(c3)S(=O)(=O)Nc3nc(cc(-c4c(C)cccc4C)n3)OC[C@H]2CC(C)(C)C)n1] | 1.75 | 699.32 | 700.8 | A |
| 1608 | 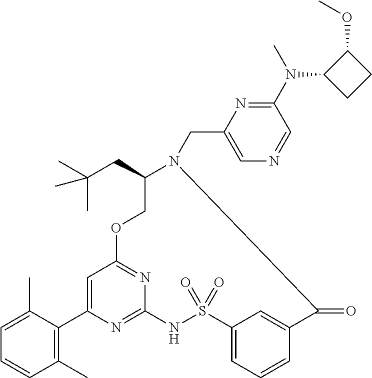 [SMILES: CO[C@@H]1CC[C@@H]1N(C)c1cncc(CN2C(=O)c3cccc(c3)S(=O)(=O)Nc3nc(cc(-c4c(C)cccc4C)n3)OC[C@H]2CC(C)(C)C)n1] | 1.76 | 699.32 | 700.8 | A |
| 1609 | 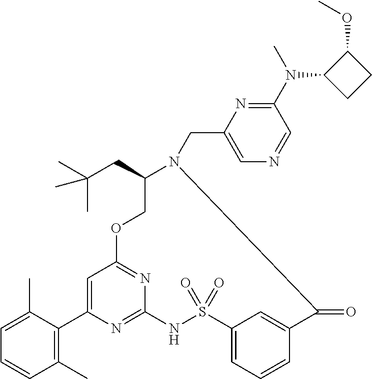 [SMILES: CO[C@@H]1CC[C@@H]1N(C)c1cncc(CN2C(=O)c3cccc(c3)S(=O)(=O)Nc3nc(cc(-c4c(C)cccc4C)n3)OC[C@H]2CC(C)(C)C)n1] | 1.75 | 699.32 | 700.9 | A |
| 1610 | 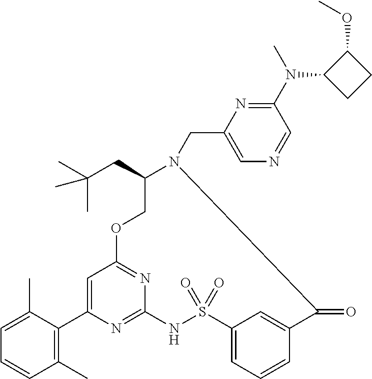 [SMILES: CO[C@@H]1CC[C@@H]1N(C)c1cncc(CN2C(=O)c3cccc(c3)S(=O)(=O)Nc3nc(cc(-c4c(C)cccc4C)n3)OC[C@H]2CC(C)(C)C)n1] | 1.76 | 699.32 | 700.8 | A |
| 1611 | 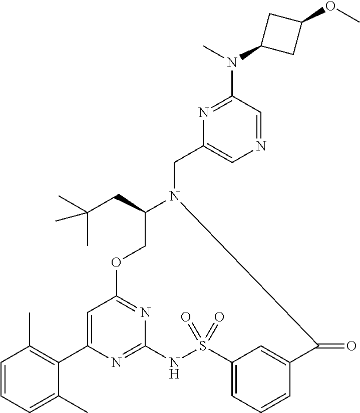 [SMILES: CO[C@H]1C[C@@H](N(C)c2cncc(CN3C(=O)c4cccc(c4)S(=O)(=O)Nc4nc(cc(-c5c(C)cccc5C)n4)OC[C@H]3CC(C)(C)C)n2)C1] | 1.57 | 699.32 | 700.5 | A |
| 1612 | 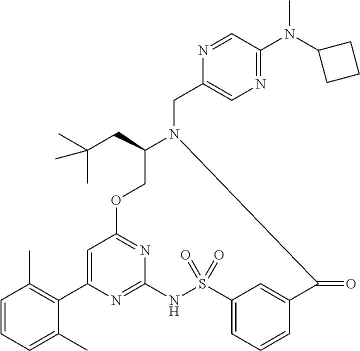 [SMILES: Cc1cccc(C)c1-c1cc2nc(n1)NS(=O)(=O)c1cccc(c1)C(=O)N(Cc1cnc(N(C)C3CCC3)cn1)[C@H](CC(C)(C)C)CO2] | 1.77 | 669.31 | 670.55 | A |
| 1613 | 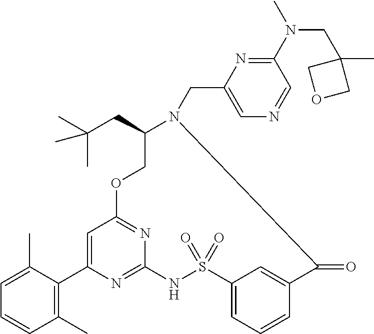 [SMILES: Cc1cccc(C)c1-c1cc2nc(n1)NS(=O)(=O)c1cccc(c1)C(=O)N(Cc1cncc(N(C)CC3(C)COC3)n1)[C@H](CC(C)(C)C)CO2] | 1.71 | 699.32 | 700.83 | A |
| 1614 | 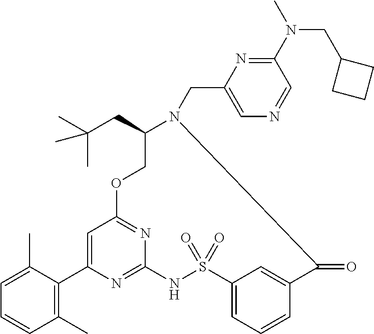 [SMILES: Cc1cccc(C)c1-c1cc2nc(n1)NS(=O)(=O)c1cccc(c1)C(=O)N(Cc1cncc(N(C)CC3CCC3)n1)[C@H](CC(C)(C)C)CO2] | 1.97 | 683.325 | 684.65 | A |
| 1615 | 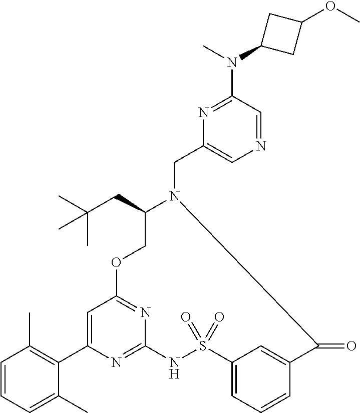 | 1.55 | 699.32 | 700.51 | A |
| 1616 | 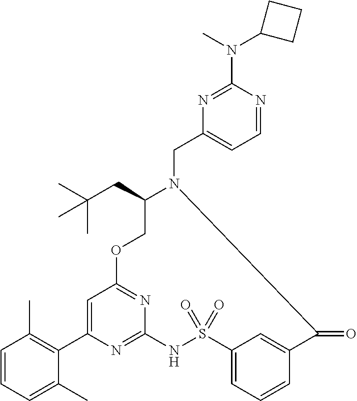 [SMILES: Cc1cccc(C)c1-c1cc2nc(n1)NS(=O)(=O)c1cccc(c1)C(=O)N(Cc1ccnc(N(C)C3CCC3)n1)[C@H](CC(C)(C)C)CO2] | 1.43 | 669.31 | 670.55 | A |
| 1617 | 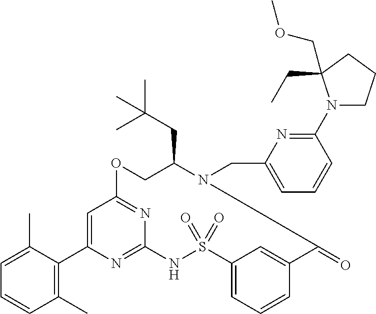 [SMILES: CC[C@@]1(COC)CCCN1c1cccc(CN2C(=O)c3cccc(c3)S(=O)(=O)Nc3nc(cc(-c4c(C)cccc4C)n3)OC[C@H]2CC(C)(C)C)n1] | 3.65 | 726.356 | 727.4 | 1D |
| 1618 | 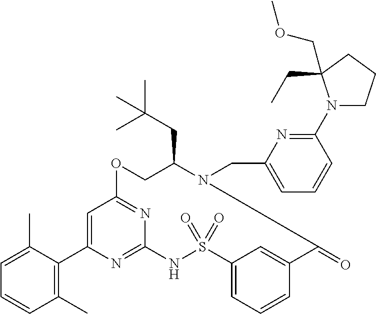 [SMILES: CC[C@@]1(COC)CCCN1c1cccc(CN2C(=O)c3cccc(c3)S(=O)(=O)Nc3nc(cc(-c4c(C)cccc4C)n3)OC[C@H]2CC(C)(C)C)n1] | 3.64 | 726.356 | 727.4 | 1D |
| 1619 | 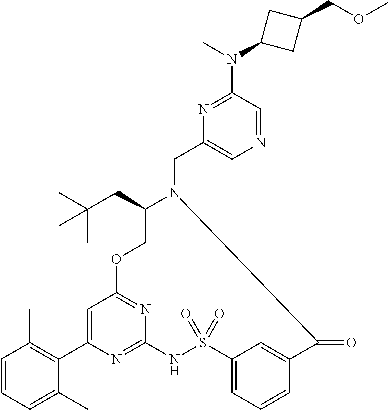 [SMILES: COC[C@H]1C[C@@H](N(C)c2cncc(CN3C(=O)c4cccc(c4)S(=O)(=O)Nc4nc(cc(-c5c(C)cccc5C)n4)OC[C@H]3CC(C)(C)C)n2)C1] | 1.59 | 713.336 | 714.56 | A |
| 1620 | 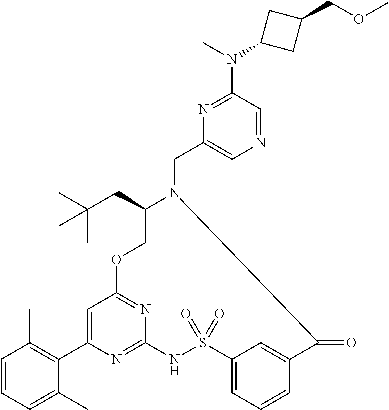 [SMILES: COC[C@H]1C[C@H](N(C)c2cncc(CN3C(=O)c4cccc(c4)S(=O)(=O)Nc4nc(cc(-c5c(C)cccc5C)n4)OC[C@H]3CC(C)(C)C)n2)C1] | 1.58 | 713.336 | 714.51 | A |
| 1621 | 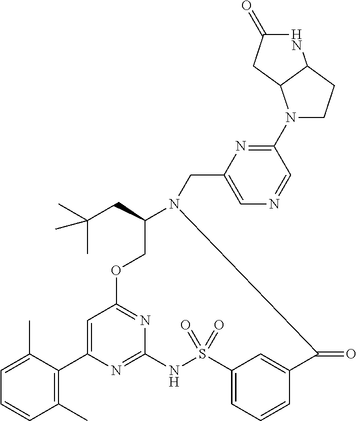 [SMILES: Cc1cccc(C)c1-c1cc2nc(n1)NS(=O)(=O)c1cccc(c1)C(=O)N(Cc1cncc(N3CCC4NC(=O)CC43)n1)[C@H](CC(C)(C)C)CO2] | 1.52 | 710.3 | 711.8 | A |
| 1622 | 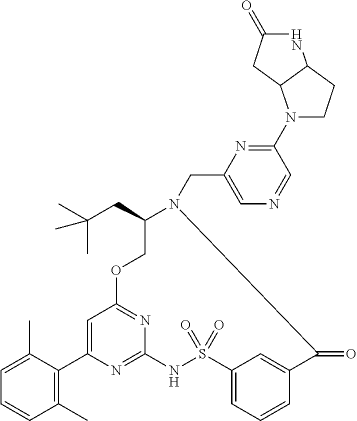 [SMILES: Cc1cccc(C)c1-c1cc2nc(n1)NS(=O)(=O)c1cccc(c1)C(=O)N(Cc1cncc(N3CCC4NC(=O)CC43)n1)[C@H](CC(C)(C)C)CO2] | 1.54 | 710.3 | 711.8 | A |
| 1623 | 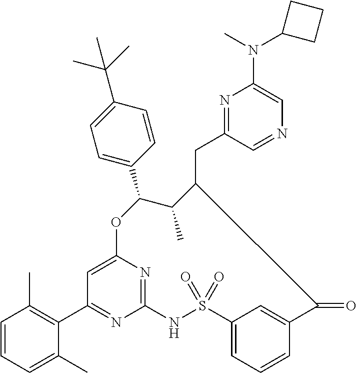 [SMILES: Cc1cccc(C)c1-c1cc2nc(n1)NS(=O)(=O)c1cccc(c1)C(=O)C(Cc1cncc(N(C)C3CCC3)n1)[C@H](C)[C@@H](c1ccc(C(C)(C)C)cc1)O2] | 2.2 | 745.341 | 746.9 | A |
| 1314 | 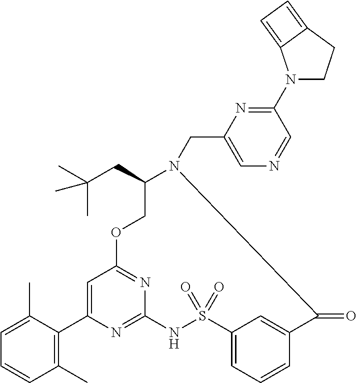 [SMILES: Cc1cccc(C)c1-c1cc2nc(n1)NS(=O)(=O)c1cccc(c1)C(=O)N(Cc1cncc(N3CCC4=CC=C43)n1)[C@H](CC(C)(C)C)CO2] | 1.9 | 681.31 | 682.9 | A |
| 1624 | 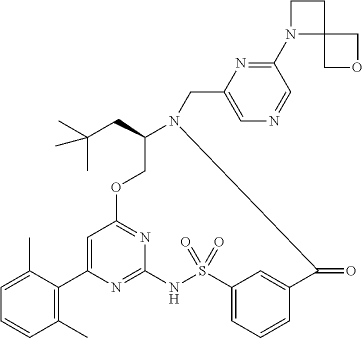 [SMILES: Cc1cccc(C)c1-c1cc2nc(n1)NS(=O)(=O)c1cccc(c1)C(=O)N(Cc1cncc(N3CCC34COC4)n1)[C@H](CC(C)(C)C)CO2] | 1.72 | 683.289 | 684.8 | A |
| 1625 | 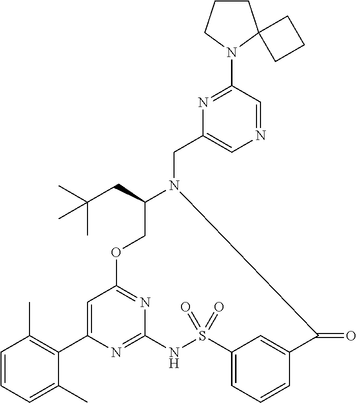 [SMILES: Cc1cccc(C)c1-c1cc2nc(n1)NS(=O)(=O)c1cccc(c1)C(=O)N(Cc1cncc(N3CCCC34CCC4)n1)[C@H](CC(C)(C)C)CO2] | 1.74 | 695.325 | 696.5 | A |
| 1626 | 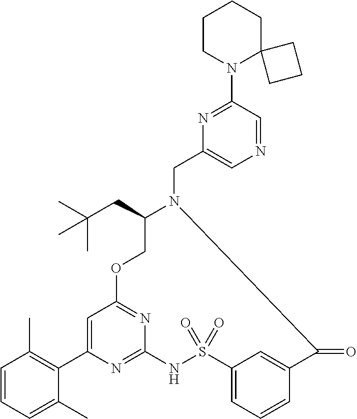 [SMILES: Cc1cccc(C)c1-c1cc2nc(n1)NS(=O)(=O)c1cccc(c1)C(=O)N(Cc1cncc(N3CCCCC34CCC4)n1)[C@H](CC(C)(C)C)CO2] | 1.77 | 709.341 | 710.5 | A |
| 1627 | 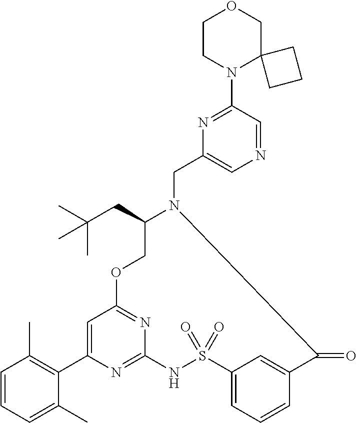 [SMILES: Cc1cccc(C)c1-c1cc2nc(n1)NS(=O)(=O)c1cccc(c1)C(=O)N(Cc1cncc(N3CCOCC34CCC4)n1)[C@H](CC(C)(C)C)CO2] | 1.63 | 711.32 | 712.3 | A |
| 1316 | 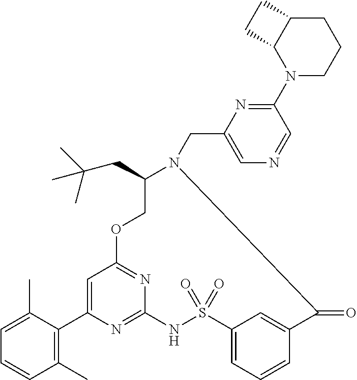 [SMILES: Cc1cccc(C)c1-c1cc2nc(n1)NS(=O)(=O)c1cccc(c1)C(=O)N(Cc1cncc(N3CCC[C@H]4CC[C@H]43)n1)[C@H](CC(C)(C)C)CO2] | 1.98 | 695.325 | 696.76 | A |
| 1315 | 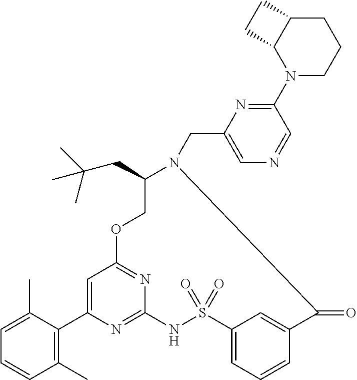 [SMILES: Cc1cccc(C)c1-c1cc2nc(n1)NS(=O)(=O)c1cccc(c1)C(=O)N(Cc1cncc(N3CCC[C@H]4CC[C@H]43)n1)[C@H](CC(C)(C)C)CO2] | 1.99 | 695.325 | 696.76 | A |
| 1628 | 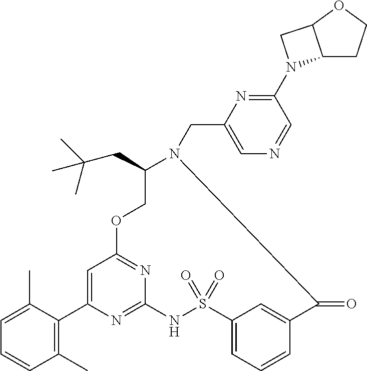 | 1.71 | 683.289 | 684.71 | A |
| 1629 | 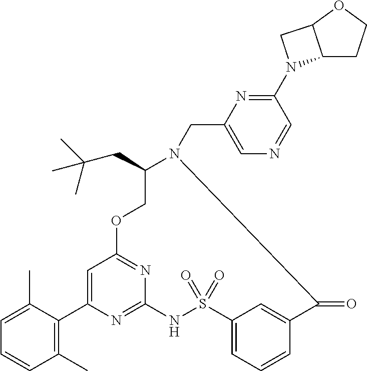 | 1.72 | 683.289 | 684.6 | A |
| 1630 | 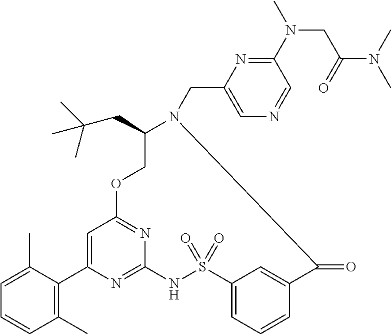 [SMILES: Cc1cccc(C)c1-c1cc2nc(n1)NS(=O)(=O)c1cccc(c1)C(=O)N(Cc1cncc(N(C)CC(=O)N(C)C)n1)[C@H](CC(C)(C)C)CO2] | 1.57 | 700.316 | 701.8 | A |
| 1631 | 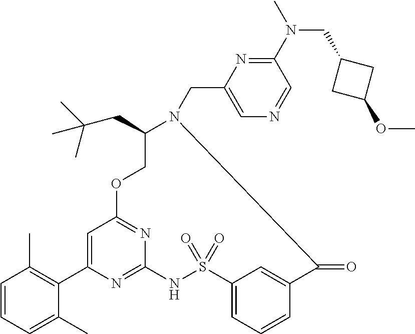 [SMILES: CO[C@H]1C[C@H](CN(C)c2cncc(CN3C(=O)c4cccc(c4)S(=O)(=O)Nc4nc(cc(-c5c(C)cccc5C)n4)OC[C@H]3CC(C)(C)C)n2)C1] | 1.79 | 713.336 | 714.8 | A |
| 1632 | 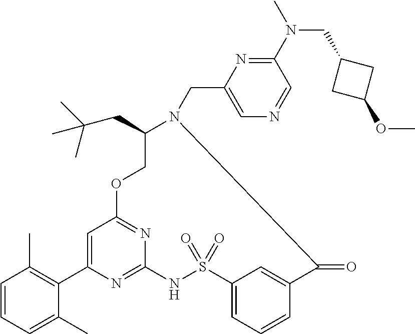 [SMILES: CO[C@H]1C[C@H](CN(C)c2cncc(CN3C(=O)c4cccc(c4)S(=O)(=O)Nc4nc(cc(-c5c(C)cccc5C)n4)OC[C@H]3CC(C)(C)C)n2)C1] | 1.79 | 713.336 | 714.9 | A |
| 1633 | 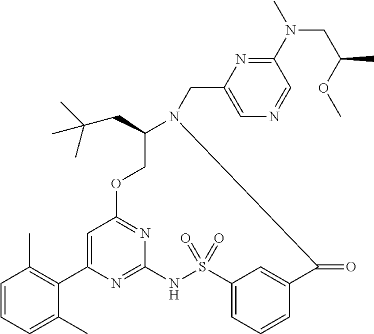 [SMILES: CO[C@H](C)CN(C)c1cncc(CN2C(=O)c3cccc(c3)S(=O)(=O)Nc3nc(cc(-c4c(C)cccc4C)n3)OC[C@H]2CC(C)(C)C)n1] | 1.75 | 687.32 | 688.7 | A |
| 1634 | 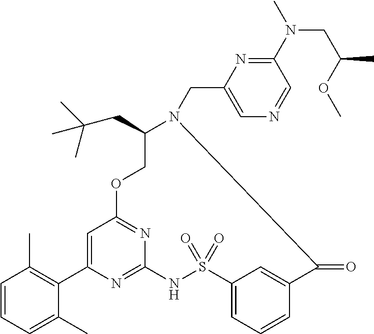 [SMILES: CO[C@H](C)CN(C)c1cncc(CN2C(=O)c3cccc(c3)S(=O)(=O)Nc3nc(cc(-c4c(C)cccc4C)n3)OC[C@H]2CC(C)(C)C)n1] | 1.7 | 687.32 | 688.9 | A |
| 1635 | 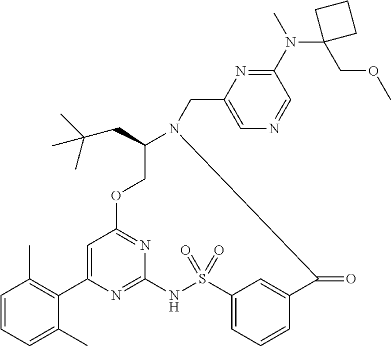 [SMILES: COCC1(N(C)c2cncc(CN3C(=O)c4cccc(c4)S(=O)(=O)Nc4nc(cc(-c5c(C)cccc5C)n4)OC[C@H]3CC(C)(C)C)n2)CCC1] | 1.87 | 713.336 | 714.9 | A |
| 1636 | 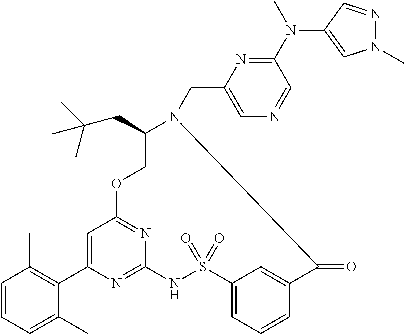 [SMILES: Cc1cccc(C)c1-c1cc2nc(n1)NS(=O)(=O)c1cccc(c1)C(=O)N(Cc1cncc(N(C)c3cnn(C)c3)n1)[C@H](CC(C)(C)C)CO2] | 1.7 | 695.3 | 696.8 | A |
| 1637 | 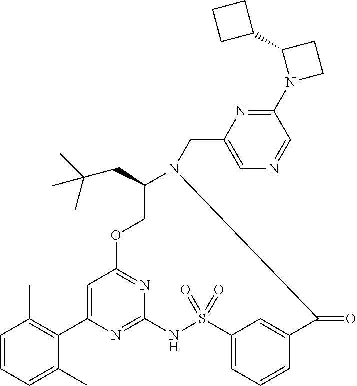 [SMILES: Cc1cccc(C)c1-c1cc2nc(n1)NS(=O)(=O)c1cccc(c1)C(=O)N(Cc1cncc(N3CC[C@H]3C3CCC3)n1)[C@H](CC(C)(C)C)CO2] | 2.05 | 695.325 | 696.8 | A |
| 1638 | 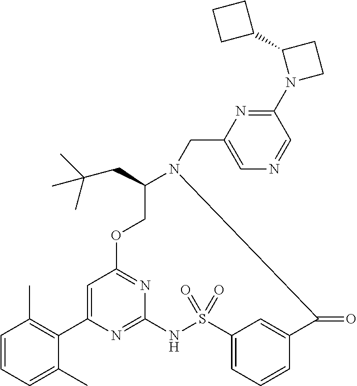 [SMILES: Cc1cccc(C)c1-c1cc2nc(n1)NS(=O)(=O)c1cccc(c1)C(=O)N(Cc1cncc(N3CC[C@H]3C3CCC3)n1)[C@H](CC(C)(C)C)CO2] | 2.05 | 695.325 | 696.9 | A |
| 1317 | 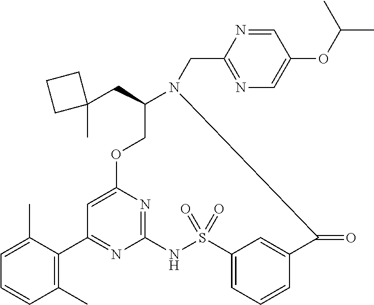 [SMILES: Cc1cccc(C)c1-c1cc2nc(n1)NS(=O)(=O)c1cccc(c1)C(=O)N(Cc1ncc(OC(C)C)cn1)[C@H](CC1(C)CCC1)CO2] | 1.96 | 656.278 | 657.71 | A |
| 1639 | 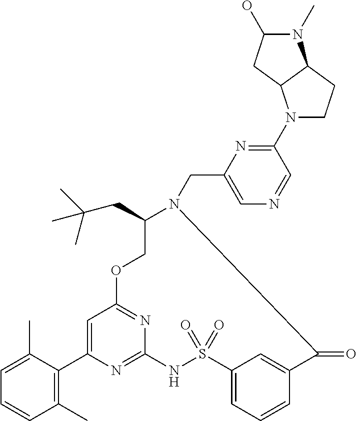 | 1.55 | 724.316 | 725.7 | A |
| 1640 | 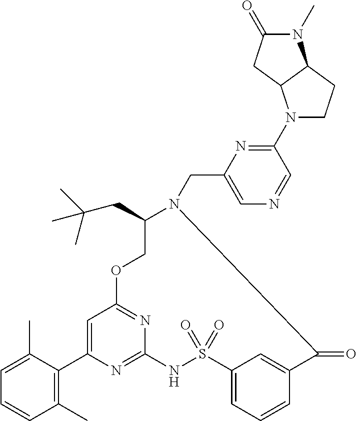 | 1.58 | 724.316 | 725.7 | A |
| 1641 | 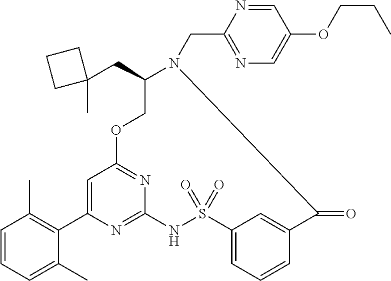 [SMILES: CCCOc1cnc(CN2C(=O)c3cccc(c3)S(=O)(=O)Nc3nc(cc(-c4c(C)cccc4C)n3)OC[C@H]2CC2(C)CCC2)nc1] | 2 | 656.278 | 657.87 | A |
| 1642 | 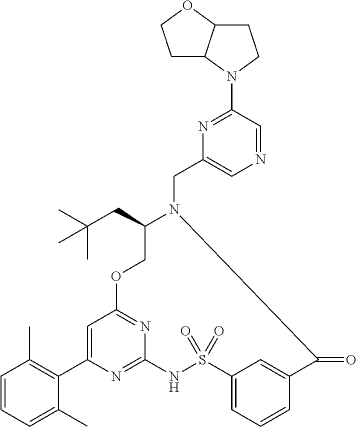 [SMILES: Cc1cccc(C)c1-c1cc2nc(n1)NS(=O)(=O)c1cccc(c1)C(=O)N(Cc1cncc(N3CCC4OCCC43)n1)[C@H](CC(C)(C)C)CO2] | 1.48 | 697.305 | 698.6 | A |
| 1643 | 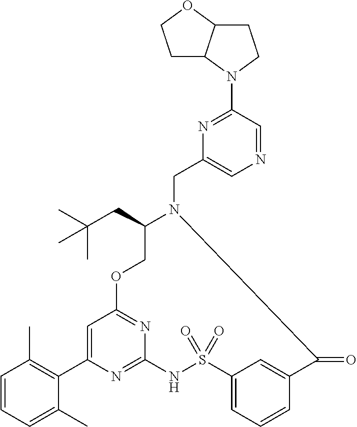 [SMILES: Cc1cccc(C)c1-c1cc2nc(n1)NS(=O)(=O)c1cccc(c1)C(=O)N(Cc1cncc(N3CCC4OCCC43)n1)[C@H](CC(C)(C)C)CO2] | 1.48 | 697.305 | 698.5 | A |
| 1644 | 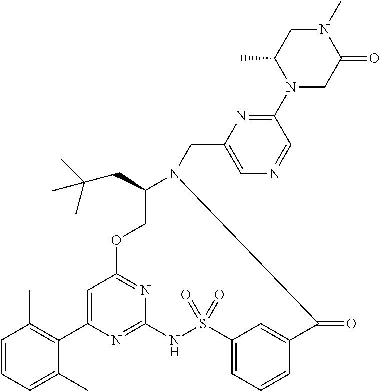 [SMILES: Cc1cccc(C)c1-c1cc2nc(n1)NS(=O)(=O)c1cccc(c1)C(=O)N(Cc1cncc(N3CC(=O)N(C)C[C@H]3C)n1)[C@H](CC(C)(C)C)CO2] | 1.59 | 712.316 | 713.8 | A |
| 1318 | 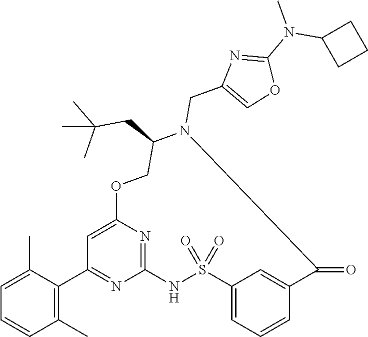 [SMILES: Cc1cccc(C)c1-c1cc2nc(n1)NS(=O)(=O)c1cccc(c1)C(=O)N(Cc1coc(N(C)C3CCC3)n1)[C@H](CC(C)(C)C)CO2] | 4.65 | 658.294 | 659.3 | Y |
| 1645 | 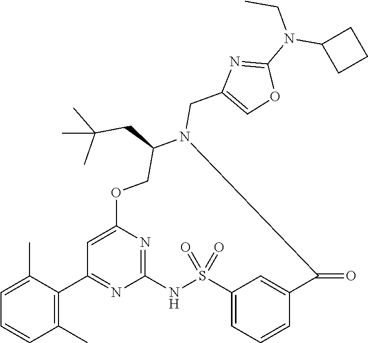 [SMILES: CCN(c1nc(CN2C(=O)c3cccc(c3)S(=O)(=O)Nc3nc(cc(-c4c(C)cccc4C)n3)OC[C@H]2CC(C)(C)C)co1)C1CCC1] | 4.85 | 672.309 | 673.3 | Y |
| 1646 | 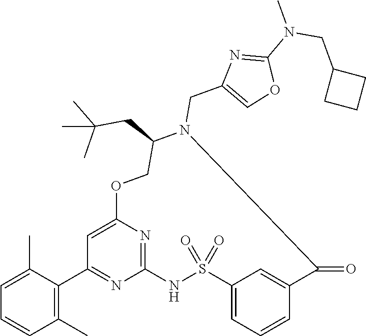 [SMILES: Cc1cccc(C)c1-c1cc2nc(n1)NS(=O)(=O)c1cccc(c1)C(=O)N(Cc1coc(N(C)CC3CCC3)n1)[C@H](CC(C)(C)C)CO2] | 4.79 | 672.309 | 673.4 | Y |
| 1647 | 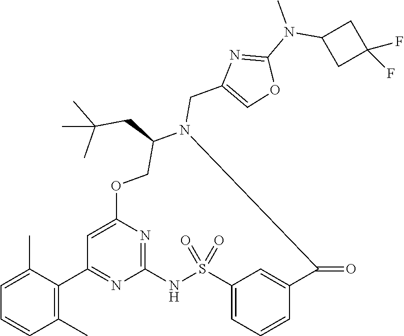 [SMILES: Cc1cccc(C)c1-c1cc2nc(n1)NS(=O)(=O)c1cccc(c1)C(=O)N(Cc1coc(N(C)C3CC(F)(F)C3)n1)[C@H](CC(C)(C)C)CO2] | 4.57 | 694.275 | 695.3 | Y |
| 1648 | 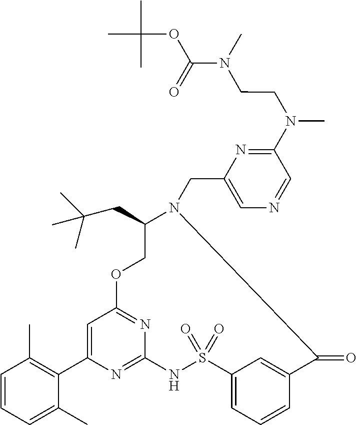 [SMILES: Cc1cccc(C)c1-c1cc2nc(n1)NS(=O)(=O)c1cccc(c1)C(=O)N(Cc1cncc(N(C)CCN(C)C(=O)OC(C)(C)C)n1)[C@H](CC(C)(C)C)CO2] | 1.91 | 772.373 | 773.9 | A |
| 1649 | 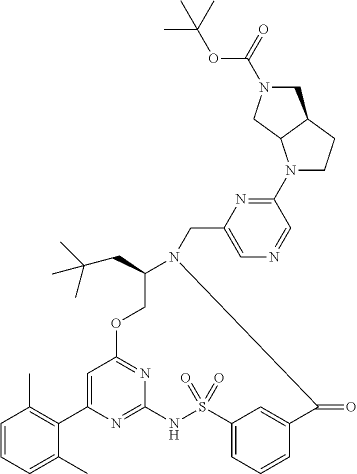 | 1.93 | 796.373 | 797.9 | A |
| 1650 | 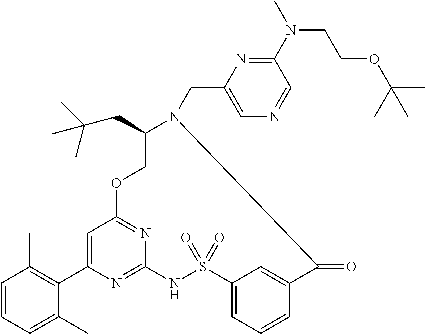 [SMILES: Cc1cccc(C)c1-c1cc2nc(n1)NS(=O)(=O)c1cccc(c1)C(=O)N(Cc1cncc(N(C)CCOC(C)(C)C)n1)[C@H](CC(C)(C)C)CO2] | 1.89 | 715.352 | 716.9 | A |
| 1651 | 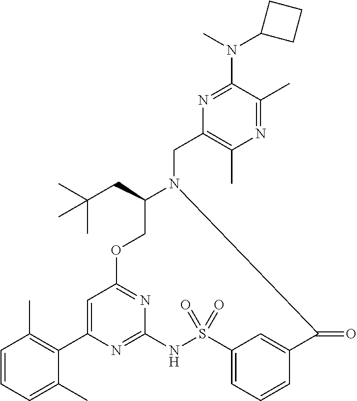 [SMILES: Cc1cccc(C)c1-c1cc2nc(n1)NS(=O)(=O)c1cccc(c1)C(=O)N(Cc1nc(N(C)C3CCC3)c(C)nc1C)[C@H](CC(C)(C)C)CO2] | 1.52 | 697.341 | 698.5 | A |
| 1652 | 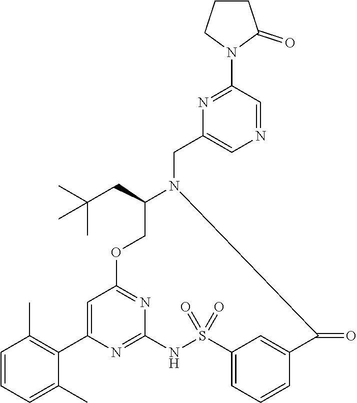 [SMILES: Cc1cccc(C)c1-c1cc2nc(n1)NS(=O)(=O)c1cccc(c1)C(=O)N(Cc1cncc(N3CCCC3=O)n1)[C@H](CC(C)(C)C)CO2] | 1.48 | 669.273 | 670.4 | A |
| 1653 | 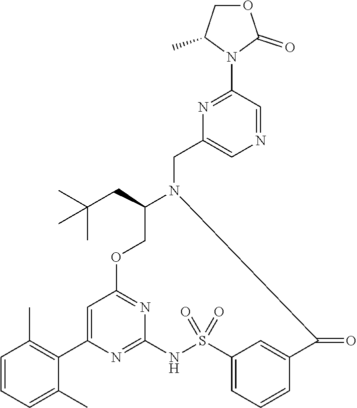 [SMILES: Cc1cccc(C)c1-c1cc2nc(n1)NS(=O)(=O)c1cccc(c1)C(=O)N(Cc1cncc(N3C(=O)OC[C@H]3C)n1)[C@H](CC(C)(C)C)CO2] | 1.52 | 685.268 | 686.4 | A |
| 1654 | 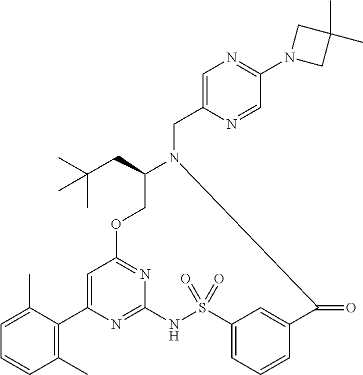 [SMILES: Cc1cccc(C)c1-c1cc2nc(n1)NS(=O)(=O)c1cccc(c1)C(=O)N(Cc1cnc(N3CC(C)(C)C3)cn1)[C@H](CC(C)(C)C)CO2] | 1.79 | 669.31 | 670.3 | A |
| 1655 | 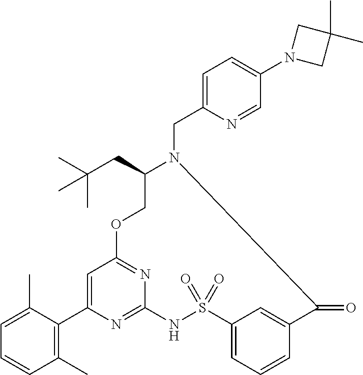 [SMILES: Cc1cccc(C)c1-c1cc2nc(n1)NS(=O)(=O)c1cccc(c1)C(=O)N(Cc1ccc(N3CC(C)(C)C3)cn1)[C@H](CC(C)(C)C)CO2] | 1.5 | 668.314 | 669.3 | A |
| 1656 | 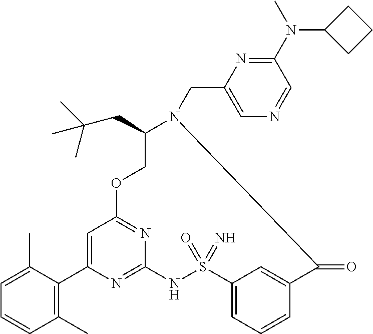 [SMILES: Cc1cccc(C)c1-c1cc2nc(n1)NS(=N)(=O)c1cccc(c1)C(=O)N(Cc1cncc(N(C)C3CCC3)n1)[C@H](CC(C)(C)C)CO2] | 1.7 | 668.326 | 669.38 | A |
| 1657 | 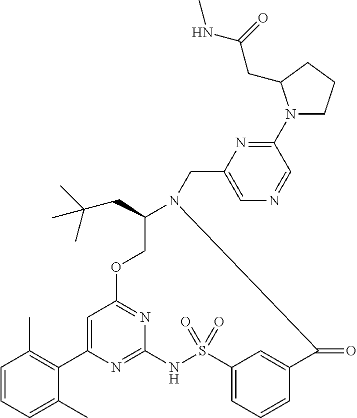 [SMILES: CNC(=O)CC1CCCN1c1cncc(CN2C(=O)c3cccc(c3)S(=O)(=O)Nc3nc(cc(-c4c(C)cccc4C)n3)OC[C@H]2CC(C)(C)C)n1] | 1.58 | 726.331 | 727.9 | A |
| 1658 | 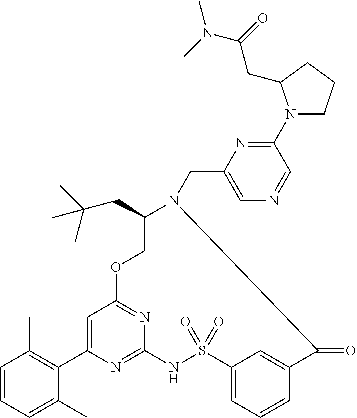 [SMILES: Cc1cccc(C)c1-c1cc2nc(n1)NS(=O)(=O)c1cccc(c1)C(=O)N(Cc1cncc(N3CCCC3CC(=O)N(C)C)n1)[C@H](CC(C)(C)C)CO2] | 1.65 | 740.347 | 741.8 | A |
| 1659 | 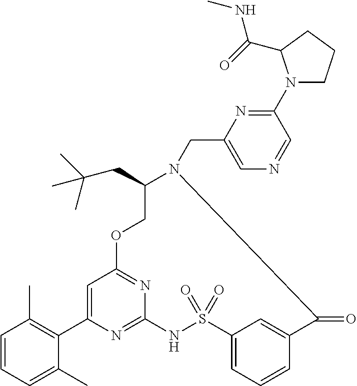 [SMILES: CNC(=O)C1CCCN1c1cncc(CN2C(=O)c3cccc(c3)S(=O)(=O)Nc3nc(cc(-c4c(C)cccc4C)n3)OC[C@H]2CC(C)(C)C)n1] | 1.56 | 712.316 | 713.8 | A |
| 1660 | 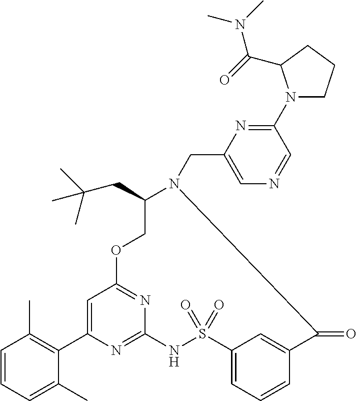 [SMILES: Cc1cccc(C)c1-c1cc2nc(n1)NS(=O)(=O)c1cccc(c1)C(=O)N(Cc1cncc(N3CCCC3C(=O)N(C)C)n1)[C@H](CC(C)(C)C)CO2] | 1.59 | 726.331 | 727.8 | A |
| 1661 | 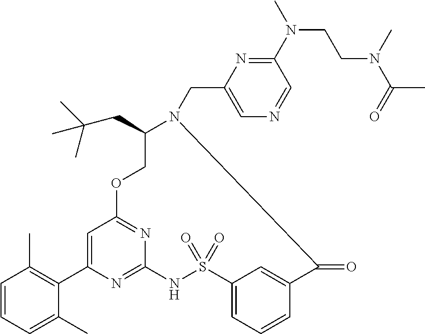 [SMILES: CC(=O)N(C)CCN(C)c1cncc(CN2C(=O)c3cccc(c3)S(=O)(=O)Nc3nc(cc(-c4c(C)cccc4C)n3)OC[C@H]2CC(C)(C)C)n1] | 1.59 | 714.331 | 715.9 | A |
| 1662 | 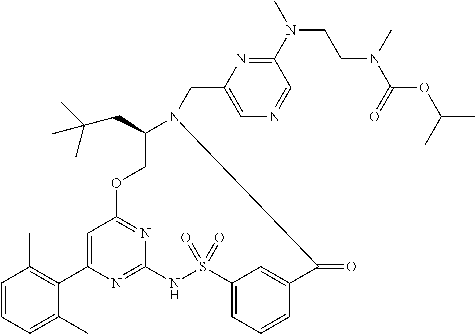 [SMILES: Cc1cccc(C)c1-c1cc2nc(n1)NS(=O)(=O)c1cccc(c1)C(=O)N(Cc1cncc(N(C)CCN(C)C(=O)OC(C)C)n1)[C@H](CC(C)(C)C)CO2] | 1.84 | 758.357 | 759.8 | A |
| 1663 | 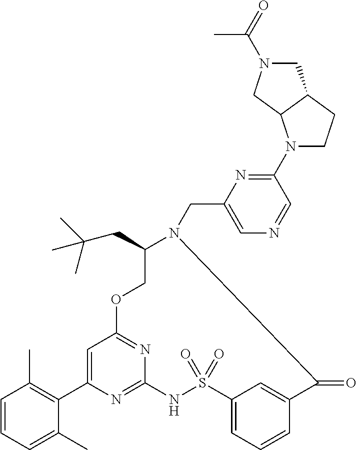 | 1.58 | 738.331 | 739.8 | A |
| 1664 | 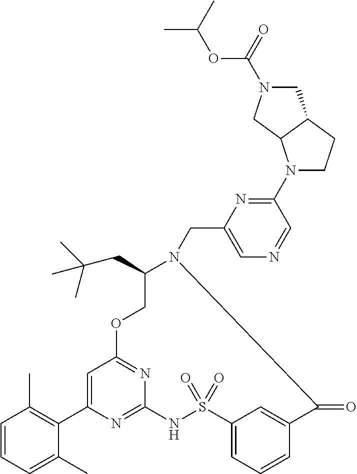 | 1.85 | 782.357 | 783.9 | A |
| 1665 | 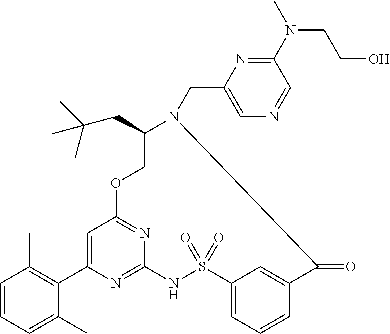 [SMILES: Cc1cccc(C)c1-c1cc2nc(n1)NS(=O)(=O)c1cccc(c1)C(=O)N(Cc1cncc(N(C)CCO)n1)[C@H](CC(C)(C)C)CO2] | 1.54 | 659.289 | 660.8 | A |
| 1666 | 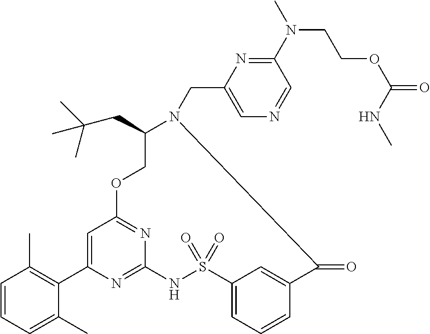 [SMILES: CNC(=O)OCCN(C)c1cncc(CN2C(=O)c3cccc(c3)S(=O)(=O)Nc3nc(cc(-c4c(C)cccc4C)n3)OC[C@H]2CC(C)(C)C)n1] | 1.62 | 716.31 | 717.7 | A |
| 1667 | 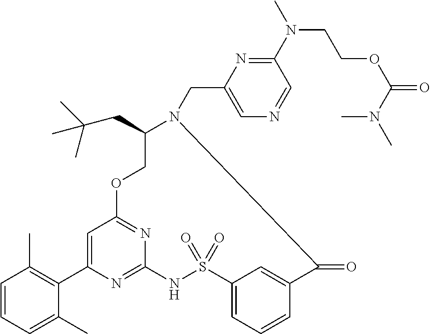 [SMILES: Cc1cccc(C)c1-c1cc2nc(n1)NS(=O)(=O)c1cccc(c1)C(=O)N(Cc1cncc(N(C)CCOC(=O)N(C)C)n1)[C@H](CC(C)(C)C)CO2] | 1.7 | 730.326 | 731.9 | A |
| 1668 | 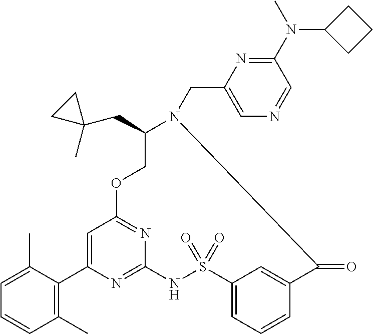 [SMILES: Cc1cccc(C)c1-c1cc2nc(n1)NS(=O)(=O)c1cccc(c1)C(=O)N(Cc1cncc(N(C)C3CCC3)n1)[C@H](CC1(C)CC1)CO2] | 1.83 | 667.294 | 668.71 | A |
| 1669 | 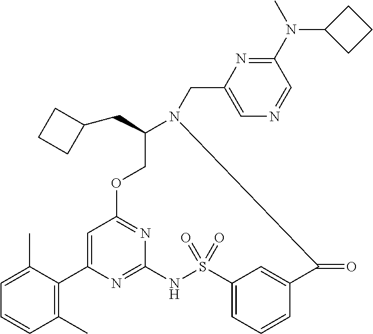 [SMILES: Cc1cccc(C)c1-c1cc2nc(n1)NS(=O)(=O)c1cccc(c1)C(=O)N(Cc1cncc(N(C)C3CCC3)n1)[C@H](CC1CCC1)CO2] | 1.88 | 667.294 | 668.77 | A |
| 1670 | 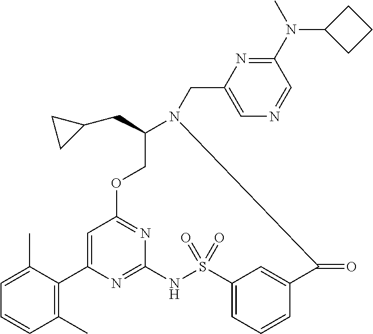 [SMILES: Cc1cccc(C)c1-c1cc2nc(n1)NS(=O)(=O)c1cccc(c1)C(=O)N(Cc1cncc(N(C)C3CCC3)n1)[C@H](CC1CC1)CO2] | 1.76 | 653.278 | 654.68 | A |
| 1671 | 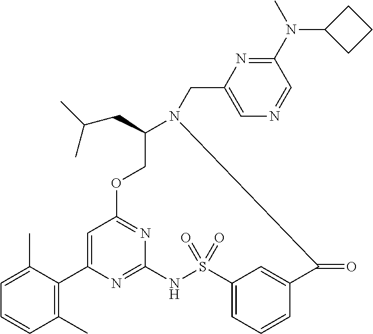 [SMILES: Cc1cccc(C)c1-c1cc2nc(n1)NS(=O)(=O)c1cccc(c1)C(=O)N(Cc1cncc(N(C)C3CCC3)n1)[C@H](CC(C)C)CO2] | 1.84 | 655.294 | 656.66 | A |
| 1672 | 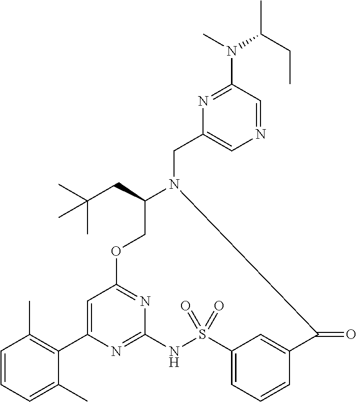 [SMILES: CC[C@@H](C)N(C)c1cncc(CN2C(=O)c3cccc(c3)S(=O)(=O)Nc3nc(cc(-c4c(C)cccc4C)n3)OC[C@H]2CC(C)(C)C)n1] | 1.6 | 671.325 | 672.4 | A |
| 1673 | 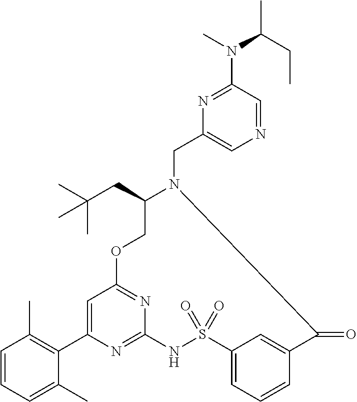 [SMILES: CC[C@H](C)N(C)c1cncc(CN2C(=O)c3cccc(c3)S(=O)(=O)Nc3nc(cc(-c4c(C)cccc4C)n3)OC[C@H]2CC(C)(C)C)n1] | 1.61 | 671.325 | 672.5 | A |
| 1674 | 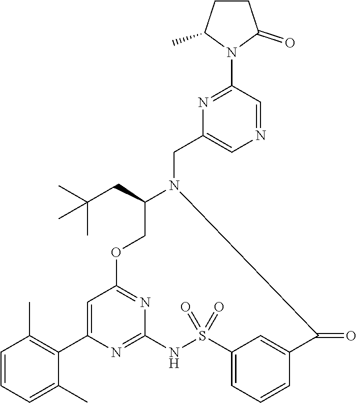 [SMILES: Cc1cccc(C)c1-c1cc2nc(n1)NS(=O)(=O)c1cccc(c1)C(=O)N(Cc1cncc(N3C(=O)CC[C@H]3C)n1)[C@H](CC(C)(C)C)CO2] | 1.53 | 683.289 | 684.4 | A |
| 1675 | 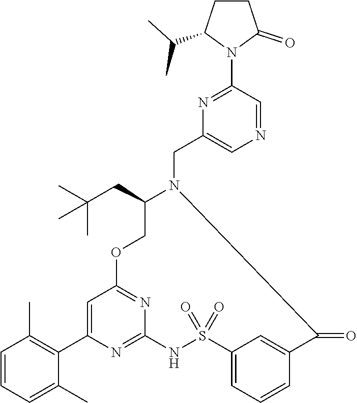 | 1.65 | 711.32 | 712.5 | A |
| 1676 | 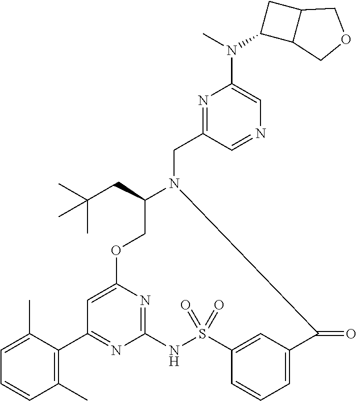 [SMILES: Cc1cccc(C)c1-c1cc2nc(n1)NS(=O)(=O)c1cccc(c1)C(=O)N(Cc1cncc(N(C)[C@@H]3CC4COCC43)n1)[C@H](CC(C)(C)C)CO2] | 1.51 | 711.32 | 712.5 | A |
| 1677 | 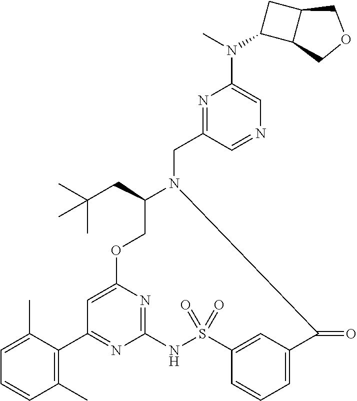 [SMILES: Cc1cccc(C)c1-c1cc2nc(n1)NS(=O)(=O)c1cccc(c1)C(=O)N(Cc1cncc(N(C)[C@@H]3C[C@H]4COC[C@H]43)n1)[C@H](CC(C)(C)C)CO2] | 1.53 | 711.32 | 712.5 | A |
| 1678 | 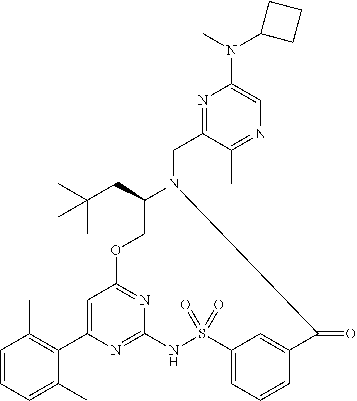 [SMILES: Cc1cccc(C)c1-c1cc2nc(n1)NS(=O)(=O)c1cccc(c1)C(=O)N(Cc1nc(N(C)C3CCC3)cnc1C)[C@H](CC(C)(C)C)CO2] | 1.81 | 683.325 | 684.5 | A |
| 1679 | 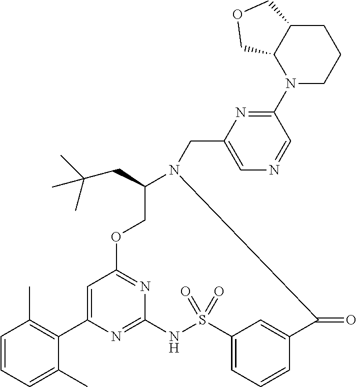 [SMILES: Cc1cccc(C)c1-c1cc2nc(n1)NS(=O)(=O)c1cccc(c1)C(=O)N(Cc1cncc(N3CCC[C@H]4COC[C@H]43)n1)[C@H](CC(C)(C)C)CO2] | 1.76 | 711.32 | 712.8 | A |
| 1680 | 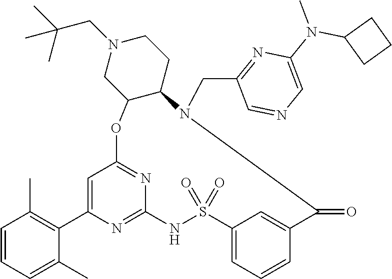 [SMILES: Cc1cccc(C)c1-c1cc2nc(n1)NS(=O)(=O)c1cccc(c1)C(=O)N(Cc1cncc(N(C)C3CCC3)n1)[C@@H]1CCN(CC(C)(C)C)CC1O2] | 1.78 | 711.32 | 712.8 | A |
| 1681 | 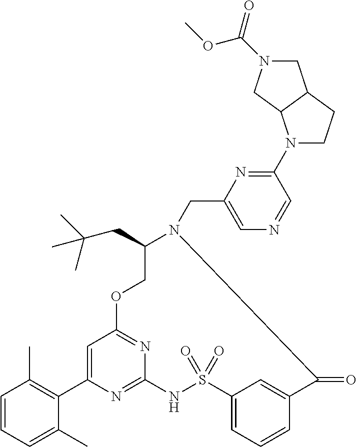 [SMILES: COC(=O)N1CC2CCN(c3cncc(CN4C(=O)c5cccc(c5)S(=O)(=O)Nc5nc(cc(-c6c(C)cccc6C)n5)OC[C@H]4CC(C)(C)C)n3)C2C1] | 1.72 | 754.326 | 755.8 | A |
| 1682 | 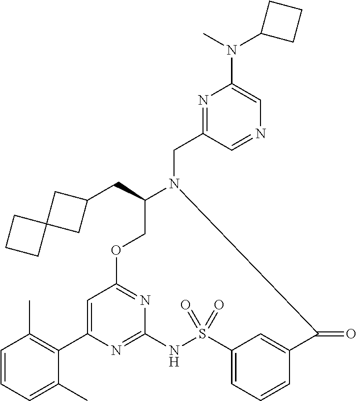 [SMILES: Cc1cccc(C)c1-c1cc2nc(n1)NS(=O)(=O)c1cccc(c1)C(=O)N(Cc1cncc(N(C)C3CCC3)n1)[C@H](CC1CC3(CCC3)C1)CO2] | 1.81 | 707.325 | 708.5 | A |
| 1683 | 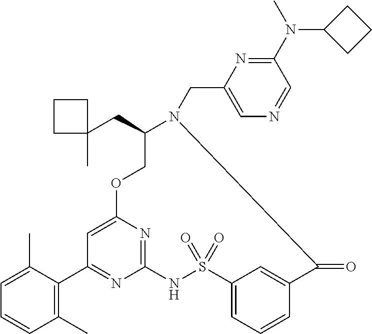 [SMILES: Cc1cccc(C)c1-c1cc2nc(n1)NS(=O)(=O)c1cccc(c1)C(=O)N(Cc1cncc(N(C)C3CCC3)n1)[C@H](CC1(C)CCC1)CO2] | 1.85 | 681.31 | 682.2 | A |
| 1684 | 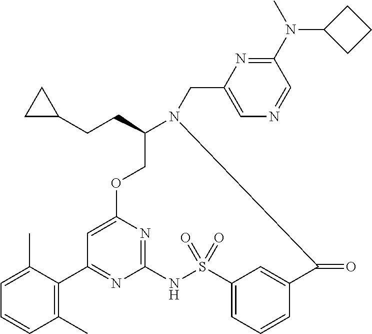 [SMILES: Cc1cccc(C)c1-c1cc2nc(n1)NS(=O)(=O)c1cccc(c1)C(=O)N(Cc1cncc(N(C)C3CCC3)n1)[C@H](CCC1CC1)CO2] | 1.79 | 667.294 | 668.2 | A |
| 1319 | 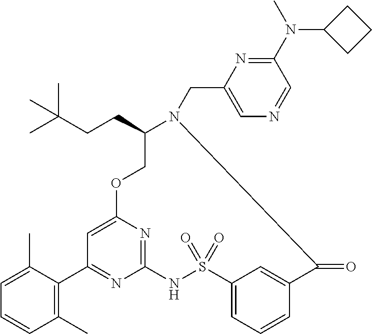 [SMILES: Cc1cccc(C)c1-c1cc2nc(n1)NS(=O)(=O)c1cccc(c1)C(=O)N(Cc1cncc(N(C)C3CCC3)n1)[C@H](CCC(C)(C)C)CO2] | 2 | 683.325 | 684.8 | A |
| 1685 | 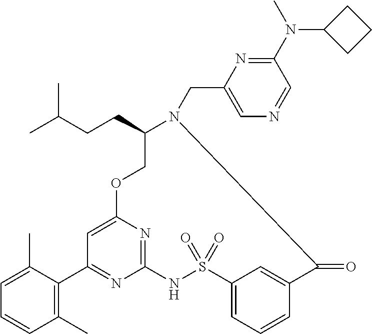 [SMILES: Cc1cccc(C)c1-c1cc2nc(n1)NS(=O)(=O)c1cccc(c1)C(=O)N(Cc1cncc(N(C)C3CCC3)n1)[C@H](CCC(C)C)CO2] | 1.86 | 669.31 | 670.2 | A |
| 1686 | 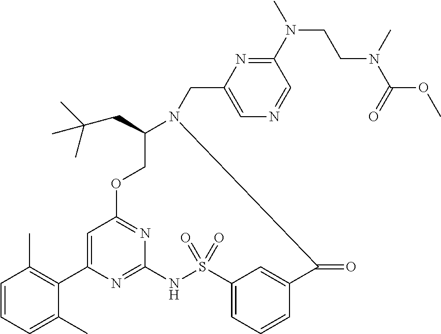 [SMILES: COC(=O)N(C)CCN(C)c1cncc(CN2C(=O)c3cccc(c3)S(=O)(=O)Nc3nc(cc(-c4c(C)cccc4C)n3)OC[C@H]2CC(C)(C)C)n1] | 1.69 | 730.326 | 731.9 | A |
| 1687 | 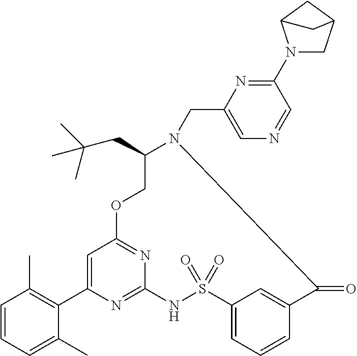 [SMILES: Cc1cccc(C)c1-c1cc2nc(n1)NS(=O)(=O)c1cccc(c1)C(=O)N(Cc1cncc(N3CC4CC3C4)n1)[C@H](CC(C)(C)C)CO2] | 1.77 | 667.294 | 668.7 | A |
| 1688 | 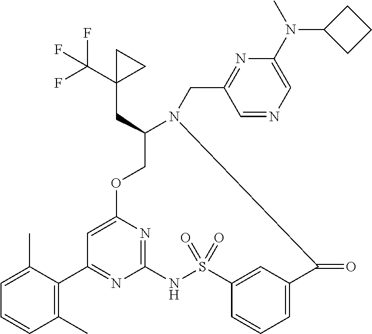 [SMILES: Cc1cccc(C)c1-c1cc2nc(n1)NS(=O)(=O)c1cccc(c1)C(=O)N(Cc1cncc(N(C)C3CCC3)n1)[C@H](CC1(C(F)(F)F)CC1)CO2] | 1.79 | 721.266 | 722.7 | A |
| 1689 | 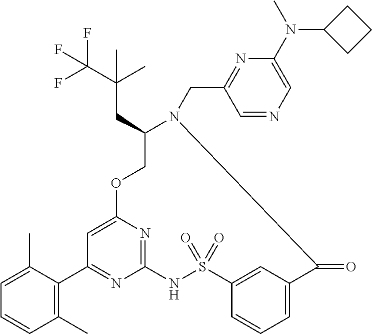 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)C(F)(F)F)CO2] | 1.86 | 723.281 | 724.7 | A |
| 1690 | 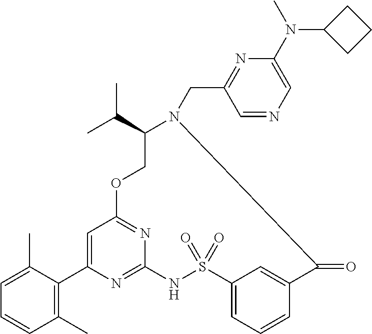 [SMILES: Cc1cccc(C)c1-c1cc2nc(n1)NS(=O)(=O)c1cccc(c1)C(=O)N(Cc1cncc(N(C)C3CCC3)n1)[C@H](C(C)C)CO2] | 1.75 | 641.278 | 642.7 | A |
| 1691 | 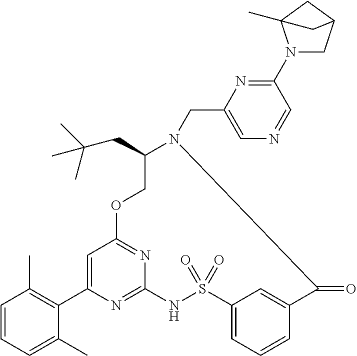 [SMILES: Cc1cccc(C)c1-c1cc2nc(n1)NS(=O)(=O)c1cccc(c1)C(=O)N(Cc1cncc(N3CC4CC3(C)C4)n1)[C@H](CC(C)(C)C)CO2] | 1.83 | 681.31 | 682.5 | A |
| 1692 | 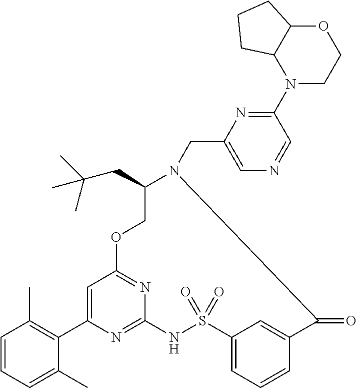 [SMILES: Cc1cccc(C)c1-c1cc2nc(n1)NS(=O)(=O)c1cccc(c1)C(=O)N(Cc1cncc(N3CCOC4CCCC43)n1)[C@H](CC(C)(C)C)CO2] | 1.82 | 711.32 | 712.8 | A |
| 1693 | 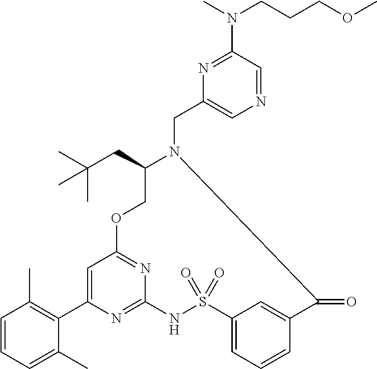 [SMILES: COCCCN(C)c1cncc(CN2C(=O)c3cccc(c3)S(=O)(=O)Nc3nc(cc(-c4c(C)cccc4C)n3)OC[C@H]2CC(C)(C)C)n1] | 1.49 | 687.32 | 688.6 | A |
| 1694 | 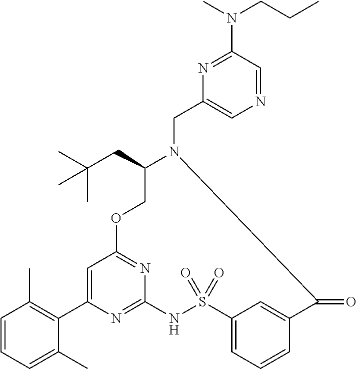 [SMILES: CCCN(C)c1cncc(CN2C(=O)c3cccc(c3)S(=O)(=O)Nc3nc(cc(-c4c(C)cccc4C)n3)OC[C@H]2CC(C)(C)C)n1] | 1.57 | 657.31 | 658.5 | A |
| 1695 | 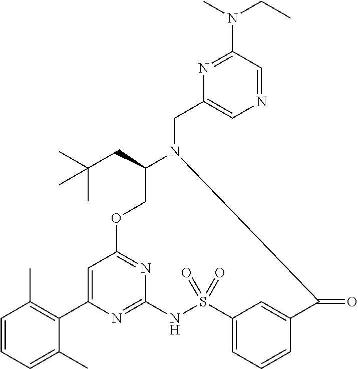 [SMILES: CCN(C)c1cncc(CN2C(=O)c3cccc(c3)S(=O)(=O)Nc3nc(cc(-c4c(C)cccc4C)n3)OC[C@H]2CC(C)(C)C)n1] | 1.48 | 643.294 | 644.4 | A |
| 1696 | 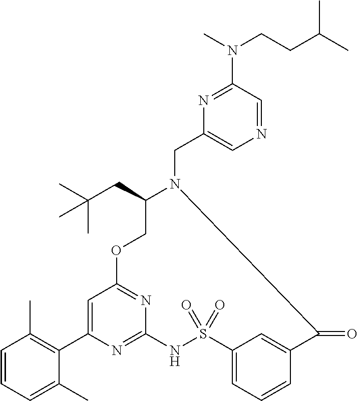 [SMILES: Cc1cccc(C)c1-c1cc2nc(n1)NS(=O)(=O)c1cccc(c1)C(=O)N(Cc1cncc(N(C)CCC(C)C)n1)[C@H](CC(C)(C)C)CO2] | 1.74 | 685.341 | 686.5 | A |
| 1697 | 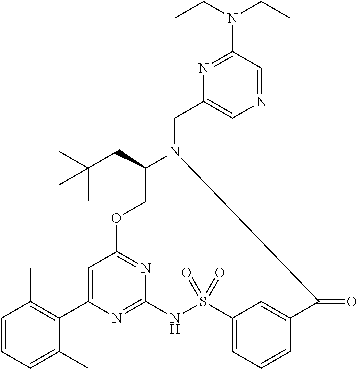 [SMILES: CCN(CC)c1cncc(CN2C(=O)c3cccc(c3)S(=O)(=O)Nc3nc(cc(-c4c(C)cccc4C)n3)OC[C@H]2CC(C)(C)C)n1] | 1.54 | 657.31 | 658.5 | A |
| 1320 | 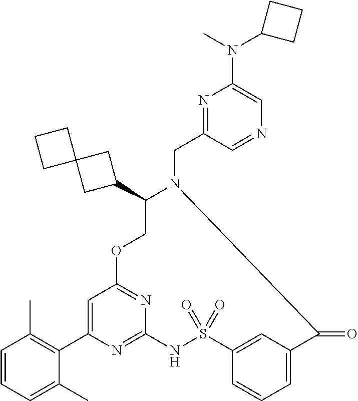 [SMILES: Cc1cccc(C)c1-c1cc2nc(n1)NS(=O)(=O)c1cccc(c1)C(=O)N(Cc1cncc(N(C)C3CCC3)n1)[C@H](C1CC3(CCC3)C1)CO2] | 1.75 | 693.31 | 694.5 | A |
| 1321 | 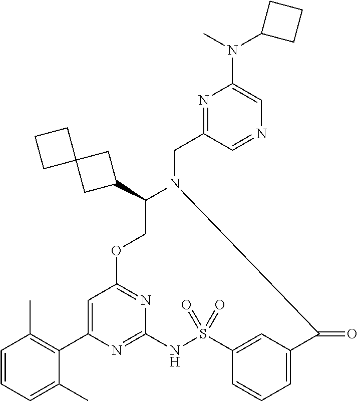 [SMILES: Cc1cccc(C)c1-c1cc2nc(n1)NS(=O)(=O)c1cccc(c1)C(=O)N(Cc1cncc(N(C)C3CCC3)n1)[C@H](C1CC3(CCC3)C1)CO2] | 1.75 | 693.31 | 694.5 | A |
| 1698 | 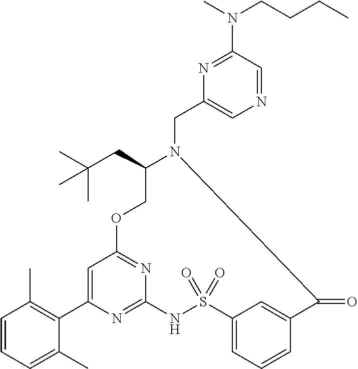 [SMILES: CCCCN(C)c1cncc(CN2C(=O)c3cccc(c3)S(=O)(=O)Nc3nc(cc(-c4c(C)cccc4C)n3)OC[C@H]2CC(C)(C)C)n1] | 1.67 | 671.325 | 672.4 | A |
| 1699 | 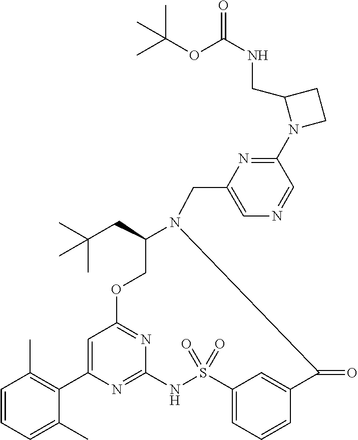 [SMILES: Cc1cccc(C)c1-c1cc2nc(n1)NS(=O)(=O)c1cccc(c1)C(=O)N(Cc1cncc(N3CCC3CNC(=O)OC(C)(C)C)n1)[C@H](CC(C)(C)C)CO2] | 2.04 | 770.357 | 771.5 | A |
| 1700 | 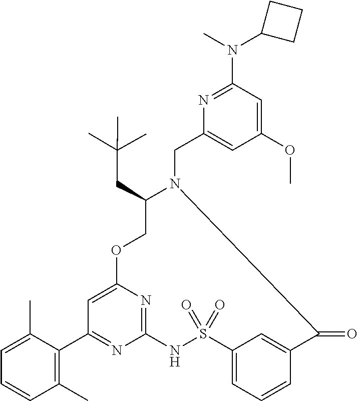 [SMILES: COc1cc(CN2C(=O)c3cccc(c3)S(=O)(=O)Nc3nc(cc(-c4c(C)cccc4C)n3)OC[C@H]2CC(C)(C)C)nc(N(C)C2CCC2)c1] | 1.65 | 698.325 | 699.5 | A |
| 1701 | 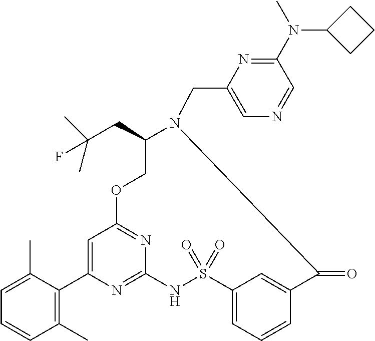 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)F)CO2] | 1.69 | 673.285 | 674.77 | A |
| 1702 | 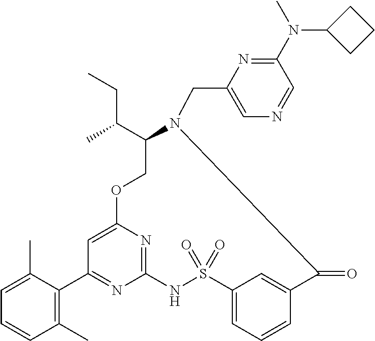 [SMILES: CC[C@@H](C)[C@@H]1COc2cc(-c3c(C)cccc3C)nc(n2)NS(=O)(=O)c2cccc(c2)C(=O)N1Cc1cncc(N(C)C2CCC2)n1] | 1.76 | 655.294 | 656.2 | A |
| 1703 | 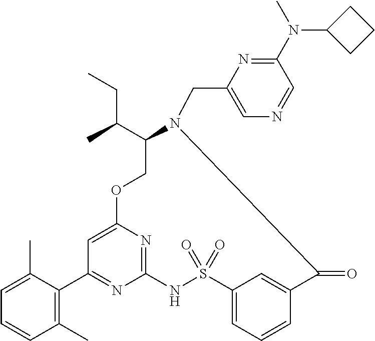 [SMILES: CC[C@H](C)[C@@H]1COc2cc(-c3c(C)cccc3C)nc(n2)NS(=O)(=O)c2cccc(c2)C(=O)N1Cc1cncc(N(C)C2CCC2)n1] | 1.76 | 655.294 | 656.2 | A |
| 1704 | 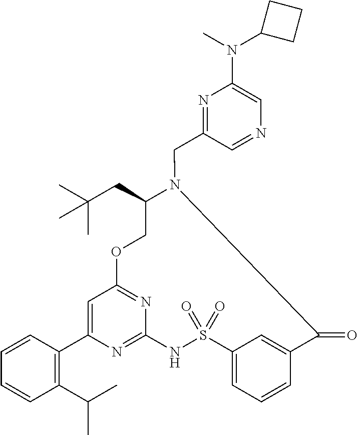 [SMILES: CC(C)c1ccccc1-c1cc2nc(n1)NS(=O)(=O)c1cccc(c1)C(=O)N(Cc1cncc(N(C)C3CCC3)n1)[C@H](CC(C)(C)C)CO2] | 1.75 | 683.325 | 684.6 | A |
| 1705 | 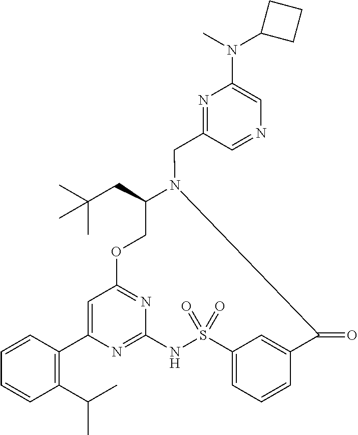 [SMILES: CC(C)c1ccccc1-c1cc2nc(n1)NS(=O)(=O)c1cccc(c1)C(=O)N(Cc1cncc(N(C)C3CCC3)n1)[C@H](CC(C)(C)C)CO2] | 1.62 | 655.294 | 656.4 | A |
| 1706 | 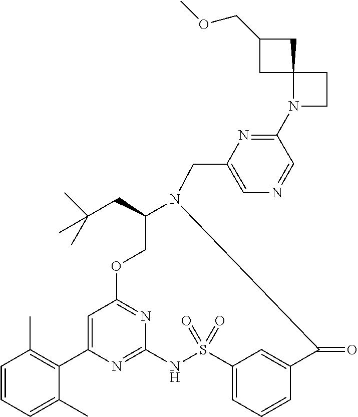 | 1.91 | 725.336 | 726.8 | A |
| 1707 | 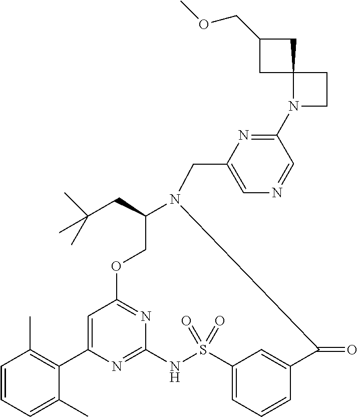 | 1.89 | 725.336 | 726.8 | A |
| 1708 | 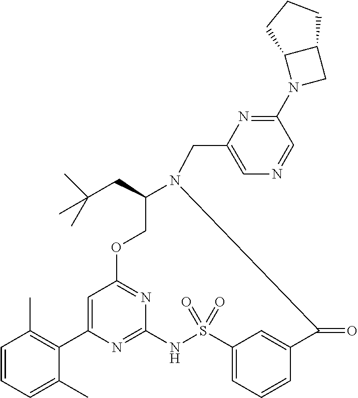 [SMILES: Cc1cccc(C)c1-c1cc2nc(n1)NS(=O)(=O)c1cccc(c1)C(=O)N(Cc1cncc(N3C[C@H]4CCC[C@H]43)n1)[C@H](CC(C)(C)C)CO2] | 1.89 | 681.31 | 682.9 | A |
| 1709 | 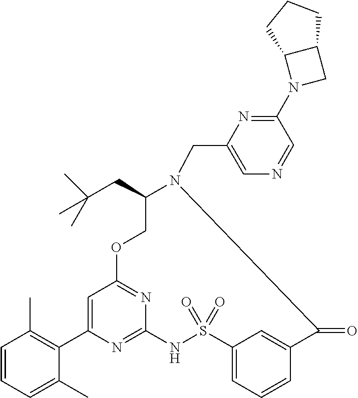 [SMILES: Cc1cccc(C)c1-c1cc2nc(n1)NS(=O)(=O)c1cccc(c1)C(=O)N(Cc1cncc(N3C[C@H]4CCC[C@H]43)n1)[C@H](CC(C)(C)C)CO2] | 1.9 | 681.31 | 682.8 | A |
| 1710 | 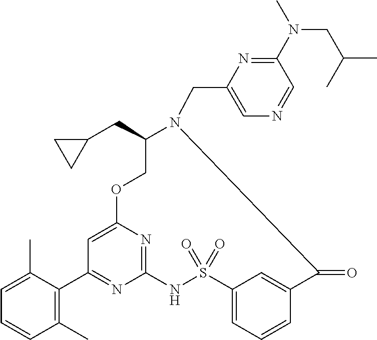 [SMILES: Cc1cccc(C)c1-c1cc2nc(n1)NS(=O)(=O)c1cccc(c1)C(=O)N(Cc1cncc(N(C)CC(C)C)n1)[C@H](CC1CC1)CO2] | 1.72 | 655.294 | 656.3 | A |
| 1711 | 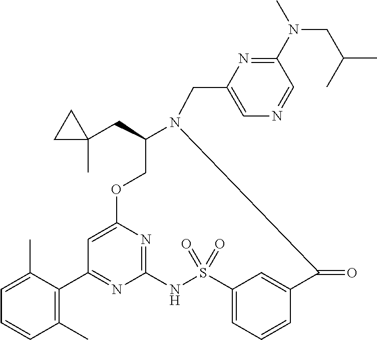 [SMILES: Cc1cccc(C)c1-c1cc2nc(n1)NS(=O)(=O)c1cccc(c1)C(=O)N(Cc1cncc(N(C)CC(C)C)n1)[C@H](CC1(C)CC1)CO2] | 1.82 | 669.31 | 670.3 | A |
| 1712 | 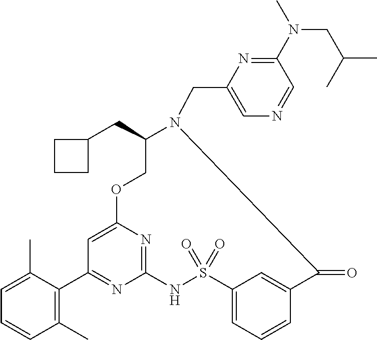 [SMILES: Cc1cccc(C)c1-c1cc2nc(n1)NS(=O)(=O)c1cccc(c1)C(=O)N(Cc1cncc(N(C)CC(C)C)n1)[C@H](CC1CCC1)CO2] | 1.78 | 669.31 | 670.3 | A |
| 1713 | 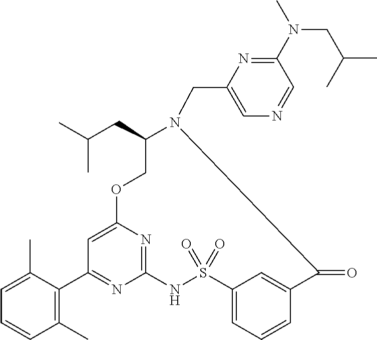 [SMILES: Cc1cccc(C)c1-c1cc2nc(n1)NS(=O)(=O)c1cccc(c1)C(=O)N(Cc1cncc(N(C)CC(C)C)n1)[C@H](CC(C)C)CO2] | 1.79 | 657.31 | 658.3 | A |
| 1714 | 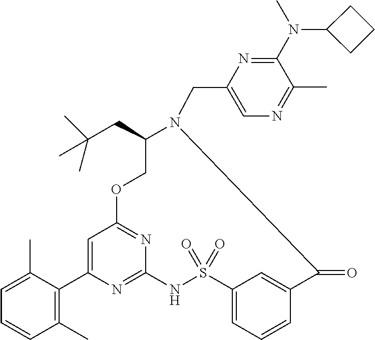 [SMILES: Cc1cccc(C)c1-c1cc2nc(n1)NS(=O)(=O)c1cccc(c1)C(=O)N(Cc1cnc(C)c(N(C)C3CCC3)n1)[C@H](CC(C)(C)C)CO2] | 1.88 | 683.325 | 684.78 | A |
| 1322 | 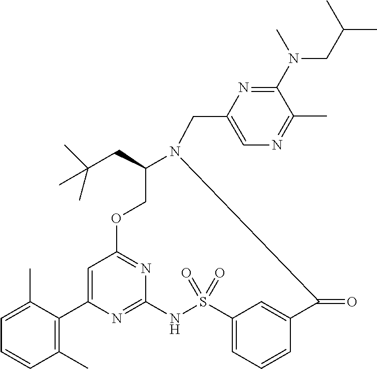 [SMILES: Cc1cccc(C)c1-c1cc2nc(n1)NS(=O)(=O)c1cccc(c1)C(=O)N(Cc1cnc(C)c(N(C)CC(C)C)n1)[C@H](CC(C)(C)C)CO2] | 1.89 | 685.341 | 686.82 | A |
| 1715 | 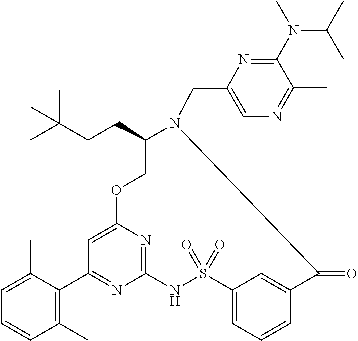 [SMILES: Cc1cccc(C)c1-c1cc2nc(n1)NS(=O)(=O)c1cccc(c1)C(=O)N(Cc1cnc(C)c(N(C)C(C)C)n1)[C@H](CCC(C)(C)C)CO2] | 1.43 | 671.325 | 672.3 | A |
| 1716 | 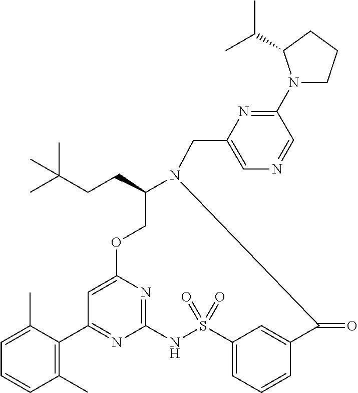 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](CCC(C)(C)C)CO2] | 2.01 | 711.357 | 712.3 | A |
| 1717 | 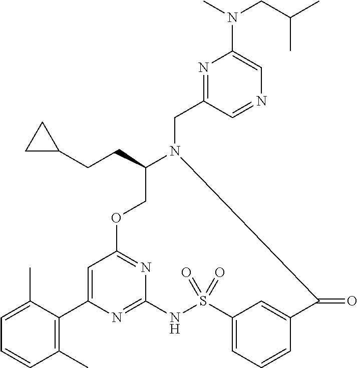 [SMILES: Cc1cccc(C)c1-c1cc2nc(n1)NS(=O)(=O)c1cccc(c1)C(=O)N(Cc1cncc(N(C)CC(C)C)n1)[C@H](CCC1CC1)CO2] | 1.62 | 669.31 | 670.4 | A |
| 1718 | 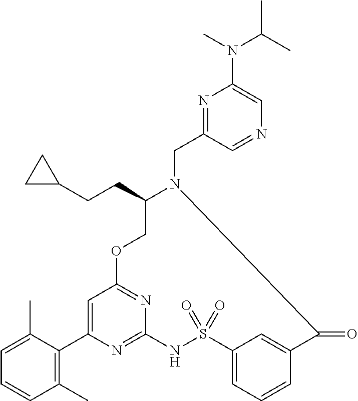 [SMILES: Cc1cccc(C)c1-c1cc2nc(n1)NS(=O)(=O)c1cccc(c1)C(=O)N(Cc1cncc(N(C)C(C)C)n1)[C@H](CCC1CC1)CO2] | 1.51 | 655.294 | 656.4 | A |
| 1719 | 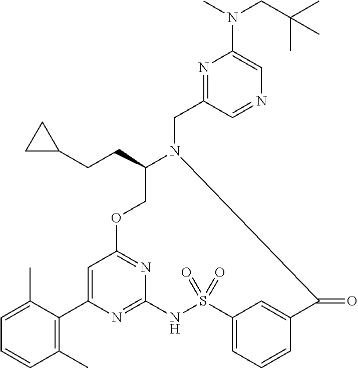 [SMILES: Cc1cccc(C)c1-c1cc2nc(n1)NS(=O)(=O)c1cccc(c1)C(=O)N(Cc1cncc(N(C)CC(C)(C)C)n1)[C@H](CCC1CC1)CO2] | 1.69 | 683.325 | 684.5 | A |
| 1720 | 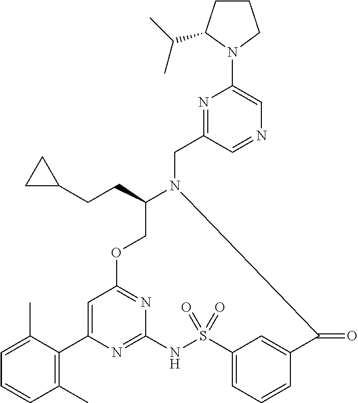 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](CCC1CC1)CO2] | 1.7 | 695.325 | 696.5 | A |
| 1721 | 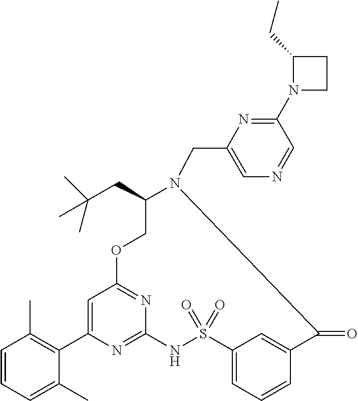 [SMILES: CC[C@@H]1CCN1c1cncc(CN2C(=O)c3cccc(c3)S(=O)(=O)Nc3nc(cc(-c4c(C)cccc4C)n3)OC[C@H]2CC(C)(C)C)n1] | 1.91 | 669.31 | 670.8 | A |
| 1722 | 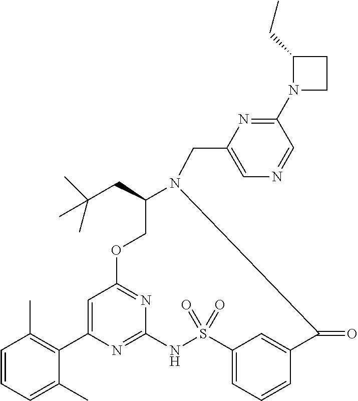 [SMILES: CC[C@@H]1CCN1c1cncc(CN2C(=O)c3cccc(c3)S(=O)(=O)Nc3nc(cc(-c4c(C)cccc4C)n3)OC[C@H]2CC(C)(C)C)n1] | 1.91 | 669.31 | 670.9 | A |
| 1723 | 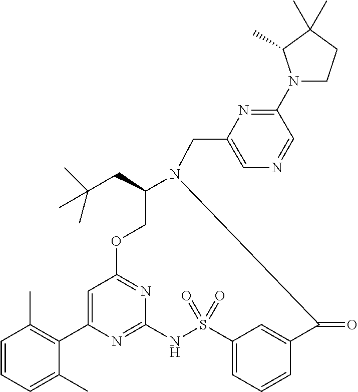 [SMILES: Cc1cccc(C)c1-c1cc2nc(n1)NS(=O)(=O)c1cccc(c1)C(=O)N(Cc1cncc(N3CCC(C)(C)[C@H]3C)n1)[C@H](CC(C)(C)C)CO2] | 1.93 | 697.341 | 698.8 | A |
| 1724 | 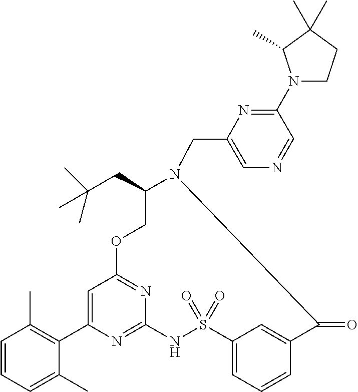 [SMILES: Cc1cccc(C)c1-c1cc2nc(n1)NS(=O)(=O)c1cccc(c1)C(=O)N(Cc1cncc(N3CCC(C)(C)[C@H]3C)n1)[C@H](CC(C)(C)C)CO2] | 1.92 | 697.341 | 698.9 | A |
| 1725 | 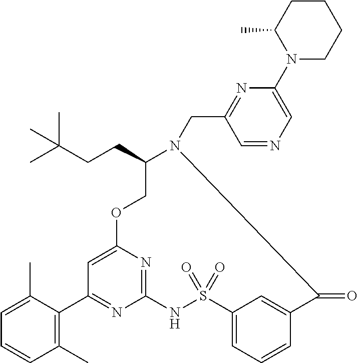 [SMILES: Cc1cccc(C)c1-c1cc2nc(n1)NS(=O)(=O)c1cccc(c1)C(=O)N(Cc1cncc(N3CCCC[C@H]3C)n1)[C@H](CCC(C)(C)C)CO2] | 2.02 | 697.341 | 698.8 | A |
| 1726 | 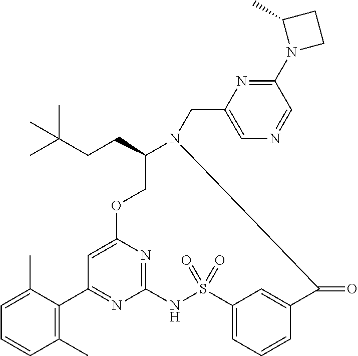 [SMILES: Cc1cccc(C)c1-c1cc2nc(n1)NS(=O)(=O)c1cccc(c1)C(=O)N(Cc1cncc(N3CC[C@H]3C)n1)[C@H](CCC(C)(C)C)CO2] | 1.95 | 669.31 | 670.9 | A |
| 1727 | 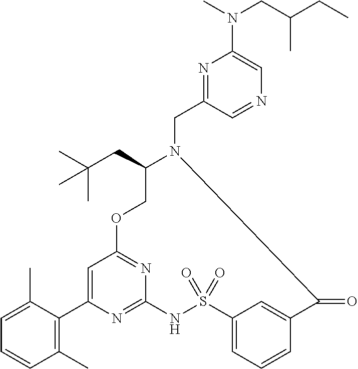 [SMILES: CCC(C)CN(C)c1cncc(CN2C(=O)c3cccc(c3)S(=O)(=O)Nc3nc(cc(-c4c(C)cccc4C)n3)OC[C@H]2CC(C)(C)C)n1] | 1.72 | 685.341 | 686.6 | A |
| 1728 | 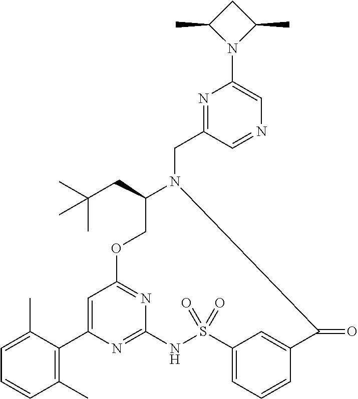 [SMILES: Cc1cccc(C)c1-c1cc2nc(n1)NS(=O)(=O)c1cccc(c1)C(=O)N(Cc1cncc(N3[C@H](C)C[C@@H]3C)n1)[C@H](CC(C)(C)C)CO2] | 1.78 | 669.31 | 670.7 | A |
| 1729 | 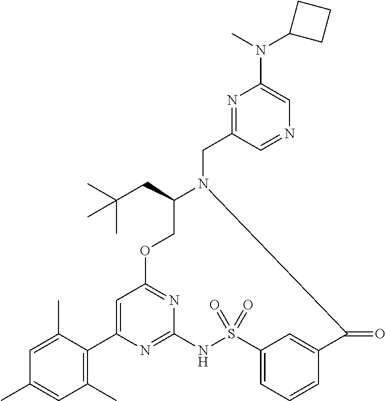 [SMILES: Cc1cc(C)c(-c2cc3nc(n2)NS(=O)(=O)c2cccc(c2)C(=O)N(Cc2cncc(N(C)C4CCC4)n2)[C@H](CC(C)(C)C)CO3)c(C)c1] | 1.71 | 683.325 | 686.4 | A |
| 1730 | 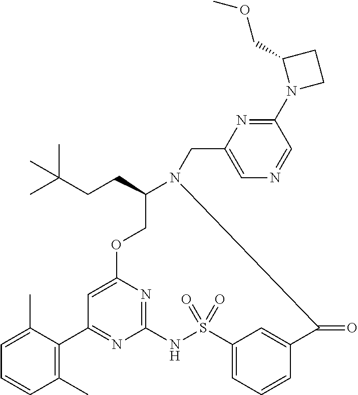 [SMILES: COC[C@@H]1CCN1c1cncc(CN2C(=O)c3cccc(c3)S(=O)(=O)Nc3nc(cc(-c4c(C)cccc4C)n3)OC[C@H]2CCC(C)(C)C)n1] | 1.9 | 699.32 | 700.8 | A |
| 1731 | 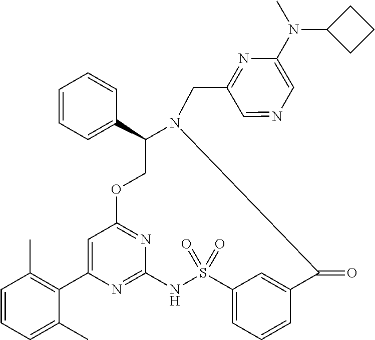 [SMILES: Cc1cccc(C)c1-c1cc2nc(n1)NS(=O)(=O)c1cccc(c1)C(=O)N(Cc1cncc(N(C)C3CCC3)n1)[C@H](c1ccccc1)CO2] | 1.76 | 675.263 | 676.8 | A |
| 1732 | 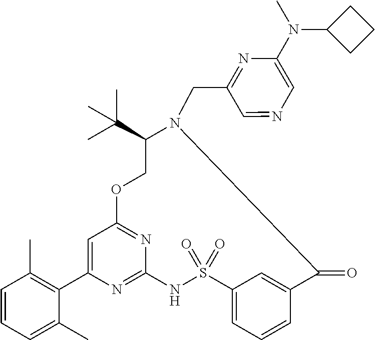 [SMILES: Cc1cccc(C)c1-c1cc2nc(n1)NS(=O)(=O)c1cccc(c1)C(=O)N(Cc1cncc(N(C)C3CCC3)n1)[C@H](C(C)(C)C)CO2] | 1.82 | 655.294 | 656.7 | A |
| 1733 | 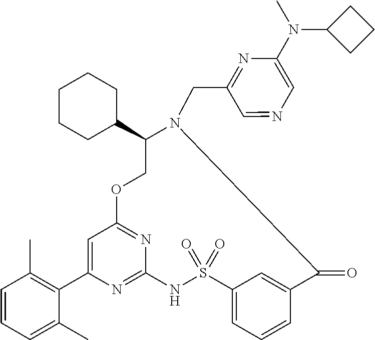 [SMILES: Cc1cccc(C)c1-c1cc2nc(n1)NS(=O)(=O)c1cccc(c1)C(=O)N(Cc1cncc(N(C)C3CCC3)n1)[C@H](C1CCCCC1)CO2] | 1.94 | 681.31 | 682.8 | A |
| 1734 | 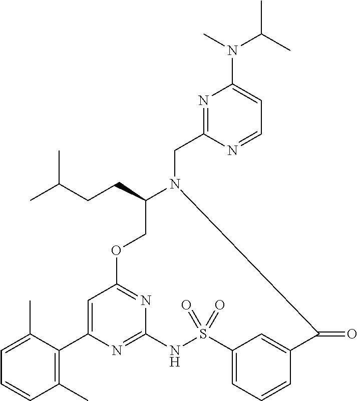 [SMILES: Cc1cccc(C)c1-c1cc2nc(n1)NS(=O)(=O)c1cccc(c1)C(=O)N(Cc1nccc(N(C)C(C)C)n1)[C@H](CCC(C)C)CO2] | 1.2 | 657.31 | 658.4 | A |
| 1735 | 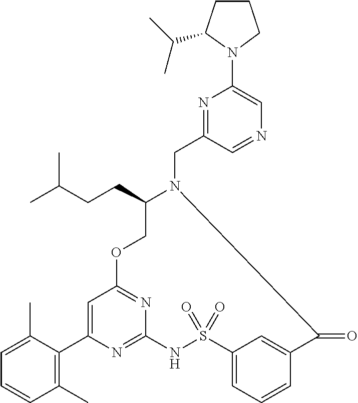 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](CCC(C)C)CO2] | 1.77 | 697.341 | 698.6 | A |
| 1736 | 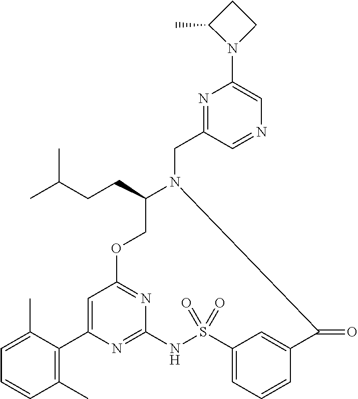 [SMILES: Cc1cccc(C)c1-c1cc2nc(n1)NS(=O)(=O)c1cccc(c1)C(=O)N(Cc1cncc(N3CC[C@H]3C)n1)[C@H](CCC(C)C)CO2] | 1.62 | 655.294 | 656.4 | A |
| 1737 | 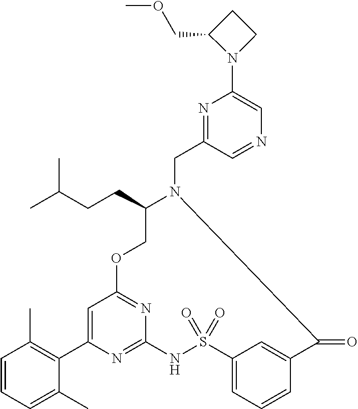 [SMILES: COC[C@@H]1CCN1c1cncc(CN2C(=O)c3cccc(c3)S(=O)(=O)Nc3nc(cc(-c4c(C)cccc4C)n3)OC[C@H]2CCC(C)C)n1] | 1.57 | 685.305 | 686.4 | A |
| 1738 | 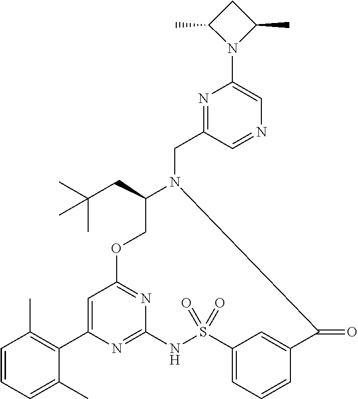 [SMILES: Cc1cccc(C)c1-c1cc2nc(n1)NS(=O)(=O)c1cccc(c1)C(=O)N(Cc1cncc(N3[C@H](C)C[C@H]3C)n1)[C@H](CC(C)(C)C)CO2] | 1.62 | 669.31 | 670.4 | A |
| 1739 | 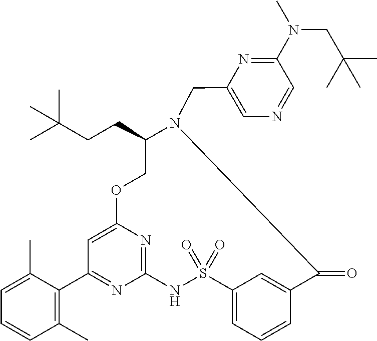 [SMILES: Cc1cccc(C)c1-c1cc2nc(n1)NS(=O)(=O)c1cccc(c1)C(=O)N(Cc1cncc(N(C)CC(C)(C)C)n1)[C@H](CCC(C)(C)C)CO2] | 2 | 699.357 | 700.3 | A |
| 1740 | 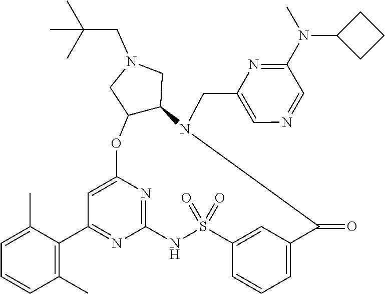 [SMILES: Cc1cccc(C)c1-c1cc2nc(n1)NS(=O)(=O)c1cccc(c1)C(=O)N(Cc1cncc(N(C)C3CCC3)n1)[C@@H]1CN(CC(C)(C)C)CC1O2] | 1.66 | 710.336 | 711.3 | A |
| 1741 | 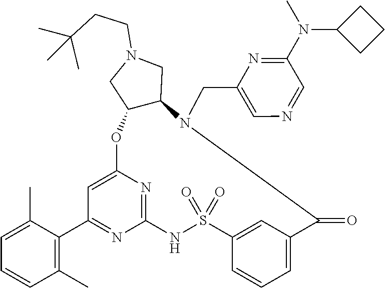 [SMILES: Cc1cccc(C)c1-c1cc2nc(n1)NS(=O)(=O)c1cccc(c1)C(=O)N(Cc1cncc(N(C)C3CCC3)n1)[C@@H]1CN(CCC(C)(C)C)C[C@H]1O2] | 1.37 | 724.352 | 725.3 | A |
| 1742 | 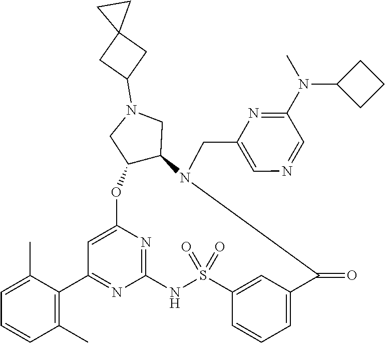 [SMILES: Cc1cccc(C)c1-c1cc2nc(n1)NS(=O)(=O)c1cccc(c1)C(=O)N(Cc1cncc(N(C)C3CCC3)n1)[C@@H]1CN(C3CC4(CC4)C3)C[C@H]1O2] | 1.31 | 720.321 | 721.3 | A |
| 1323 | 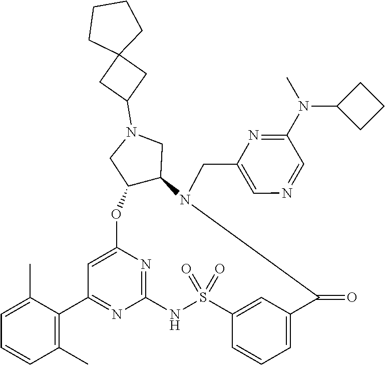 [SMILES: Cc1cccc(C)c1-c1cc2nc(n1)NS(=O)(=O)c1cccc(c1)C(=O)N(Cc1cncc(N(C)C3CCC3)n1)[C@@H]1CN(C3CC4(CCCC4)C3)C[C@H]1O2] | 1.44 | 748.352 | 749.3 | A |
| 1743 | 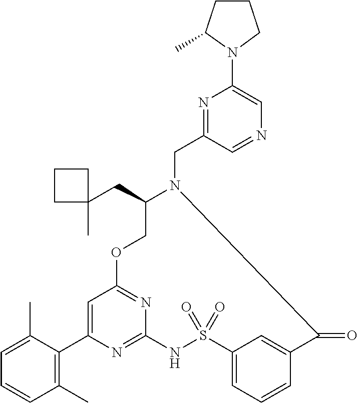 [SMILES: Cc1cccc(C)c1-c1cc2nc(n1)NS(=O)(=O)c1cccc(c1)C(=O)N(Cc1cncc(N3CCC[C@H]3C)n1)[C@H](CC1(C)CCC1)CO2] | 1.73 | 681.31 | 682.3 | A |
| 1744 | 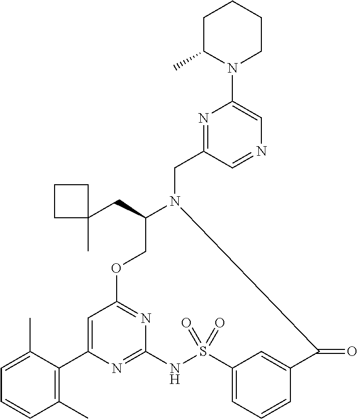 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1(C)CCC1)CO2] | 1.82 | 695.325 | 696.4 | A |
| 1745 | 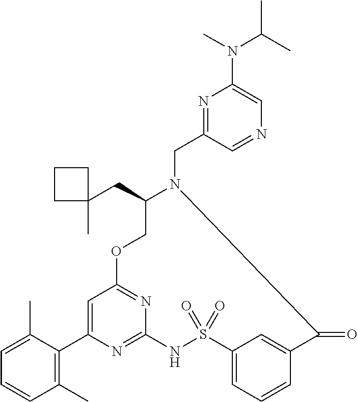 [SMILES: Cc1cccc(C)c1-c1cc2nc(n1)NS(=O)(=O)c1cccc(c1)C(=O)N(Cc1cncc(N(C)C(C)C)n1)[C@H](CC1(C)CCC1)CO2] | 1.7 | 669.31 | 670.4 | A |
| 1746 | 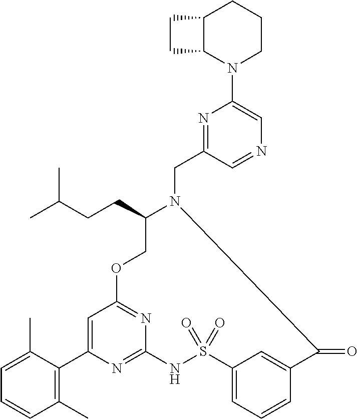 [SMILES: Cc1cccc(C)c1-c1cc2nc(n1)NS(=O)(=O)c1cccc(c1)C(=O)N(Cc1cncc(N3CCC[C@H]4CC[C@H]43)n1)[C@H](CCC(C)C)CO2] | 1.9 | 695.325 | 696.5 | A |
| 1747 | 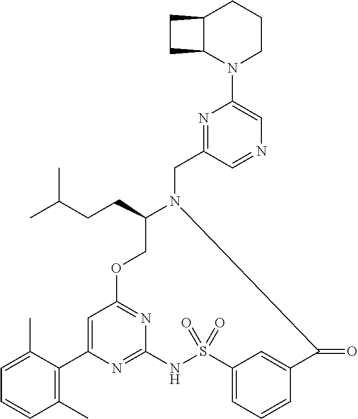 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CCC(C)C)CO2] | 1.91 | 695.325 | 696.6 | A |
| 1748 | 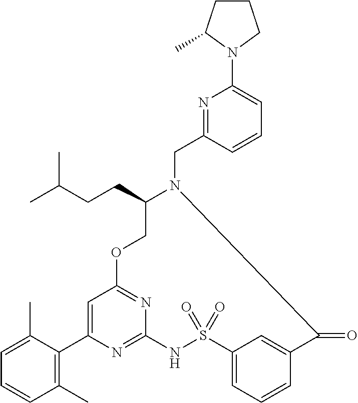 [SMILES: Cc1cccc(C)c1-c1cc2nc(n1)NS(=O)(=O)c1cccc(c1)C(=O)N(Cc1cccc(N3CCC[C@H]3C)n1)[C@H](CCC(C)C)CO2] | 1.38 | 668.314 | 669.4 | A |
| 1749 | 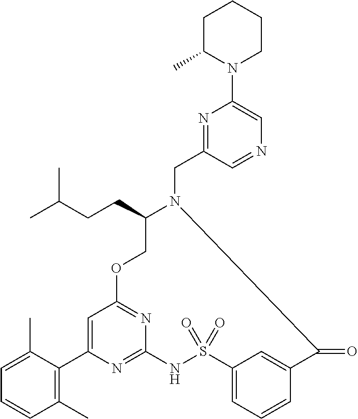 [SMILES: Cc1cccc(C)c1-c1cc2nc(n1)NS(=O)(=O)c1cccc(c1)C(=O)N(Cc1cncc(N3CCCC[C@H]3C)n1)[C@H](CCC(C)C)CO2] | 1.81 | 683.325 | 684.3 | A |
| 1750 | 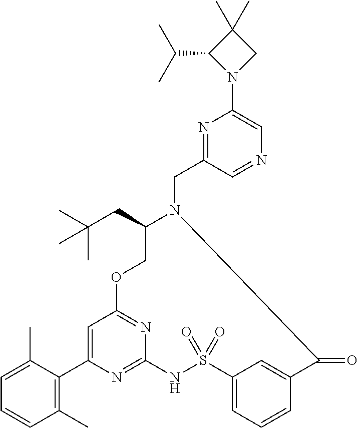 [SMILES: Cc1cccc(C)c1-c1cc2nc(n1)NS(=O)(=O)c1cccc(c1)C(=O)N(Cc1cncc(N3CC(C)(C)[C@H]3C(C)C)n1)[C@H](CC(C)(C)C)CO2] | 1.93 | 711.357 | 712.5 | A |
| 1751 | 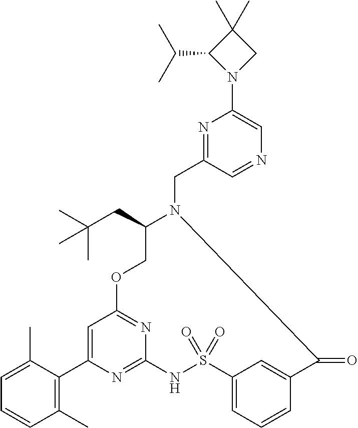 [SMILES: Cc1cccc(C)c1-c1cc2nc(n1)NS(=O)(=O)c1cccc(c1)C(=O)N(Cc1cncc(N3CC(C)(C)[C@H]3C(C)C)n1)[C@H](CC(C)(C)C)CO2] | 1.94 | 711.357 | 712.6 | A |
| 1752 | 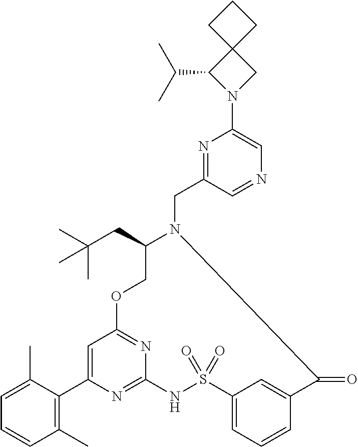 [SMILES: Cc1cccc(C)c1-c1cc2nc(n1)NS(=O)(=O)c1cccc(c1)C(=O)N(Cc1cncc(N3CC4(CCC4)[C@H]3C(C)C)n1)[C@H](CC(C)(C)C)CO2] | 1.97 | 723.357 | 724.6 | A |
| 1753 | 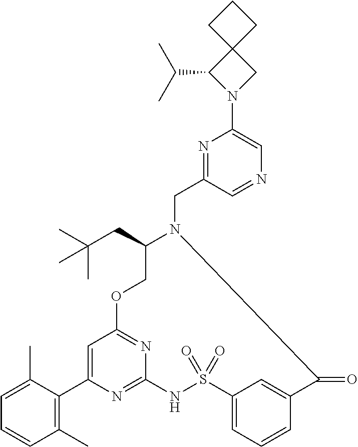 [SMILES: Cc1cccc(C)c1-c1cc2nc(n1)NS(=O)(=O)c1cccc(c1)C(=O)N(Cc1cncc(N3CC4(CCC4)[C@H]3C(C)C)n1)[C@H](CC(C)(C)C)CO2] | 2 | 723.357 | 724.5 | A |
| 1754 | 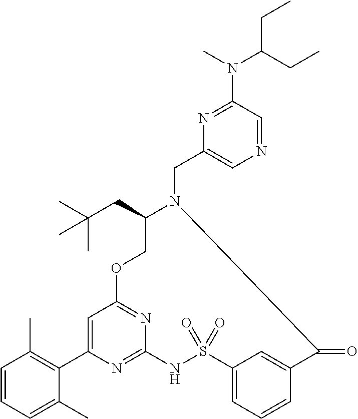 [SMILES: CCC(CC)N(C)c1cncc(CN2C(=O)c3cccc(c3)S(=O)(=O)Nc3nc(cc(-c4c(C)cccc4C)n3)OC[C@H]2CC(C)(C)C)n1] | 1.73 | 685.341 | 686.5 | A |
| 1755 | 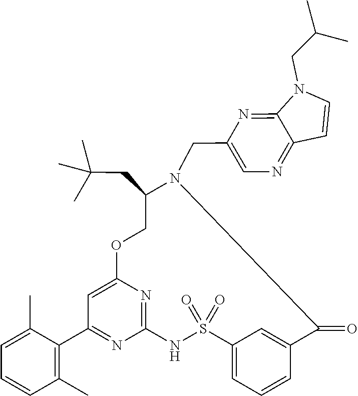 [SMILES: Cc1cccc(C)c1-c1cc2nc(n1)NS(=O)(=O)c1cccc(c1)C(=O)N(Cc1cnc3ccn(CC(C)C)c3n1)[C@H](CC(C)(C)C)CO2] | 4.36 | 681.31 | 682.3 | Y |
| 1756 | 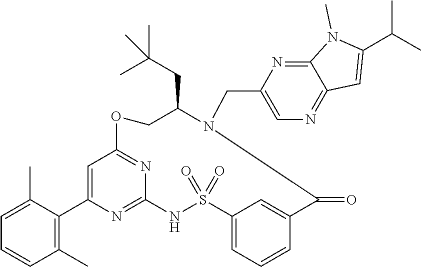 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C)CO2] | 4.61 | 681.31 | 682.4 | Y |
| 1757 | 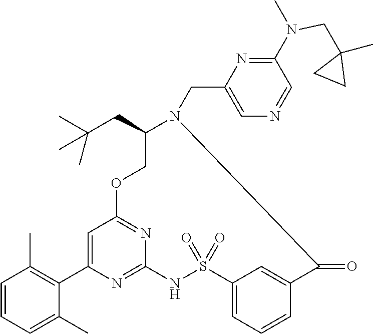 [SMILES: Cc1cccc(C)c1-c1cc2nc(n1)NS(=O)(=O)c1cccc(c1)C(=O)N(Cc1cncc(N(C)CC3(C)CC3)n1)[C@H](CC(C)(C)C)CO2] | 1.9 | 683.325 | 684.9 | A |
| 1758 | 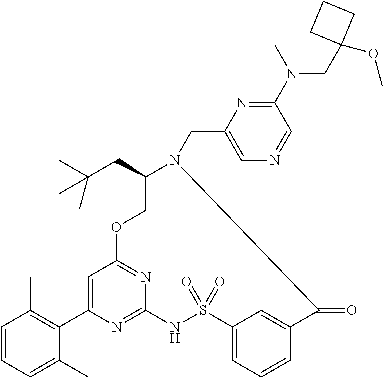 [SMILES: COC1(CN(C)c2cncc(CN3C(=O)c4cccc(c4)S(=O)(=O)Nc4nc(cc(-c5c(C)cccc5C)n4)OC[C@H]3CC(C)(C)C)n2)CCC1] | 1.85 | 713.336 | 714.8 | A |
| 1759 | 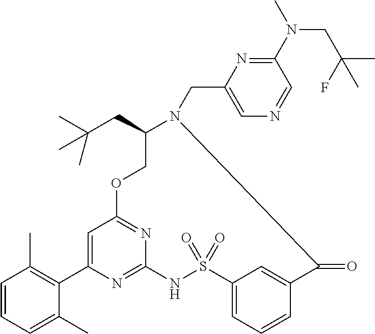 [SMILES: Cc1cccc(C)c1-c1cc2nc(n1)NS(=O)(=O)c1cccc(c1)C(=O)N(Cc1cncc(N(C)CC(C)(C)F)n1)[C@H](CC(C)(C)C)CO2] | 1.83 | 689.316 | 690.8 | A |
| 1760 | 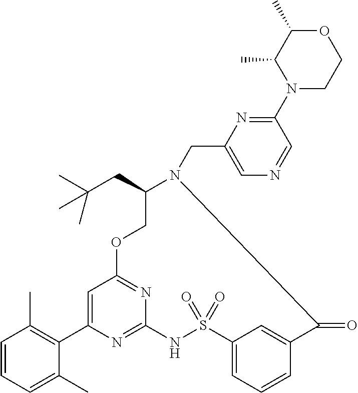 [SMILES: Cc1cccc(C)c1-c1cc2nc(n1)NS(=O)(=O)c1cccc(c1)C(=O)N(Cc1cncc(N3CCO[C@@H](C)[C@H]3C)n1)[C@H](CC(C)(C)C)CO2] | 1.76 | 699.32 | 700.8 | A |
| 1761 | 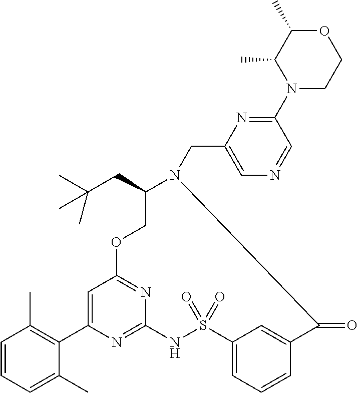 [SMILES: Cc1cccc(C)c1-c1cc2nc(n1)NS(=O)(=O)c1cccc(c1)C(=O)N(Cc1cncc(N3CCO[C@@H](C)[C@H]3C)n1)[C@H](CC(C)(C)C)CO2] | 1.86 | 699.32 | 700.8 | A |
| 1762 | 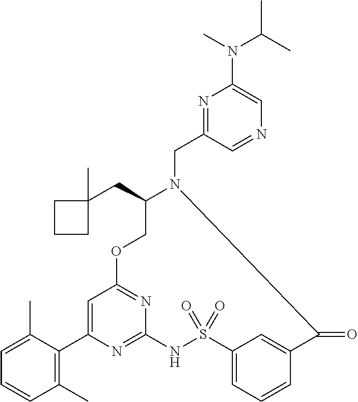 [SMILES: Cc1cccc(C)c1-c1cc2nc(n1)NS(=O)(=O)c1cccc(c1)C(=O)N(Cc1cncc(N(C)C(C)C)n1)[C@H](CC1(C)CCC1)CO2] | 1.31 | 668.314 | 669.4 | A |
| 1763 | 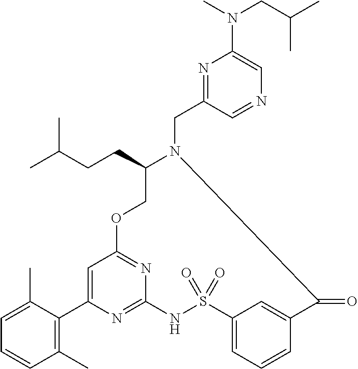 [SMILES: Cc1cccc(C)c1-c1cc2nc(n1)NS(=O)(=O)c1cccc(c1)C(=O)N(Cc1cncc(N(C)CC(C)C)n1)[C@H](CCC(C)C)CO2] | 1.7 | 671.325 | 672.5 | A |
| 1764 | 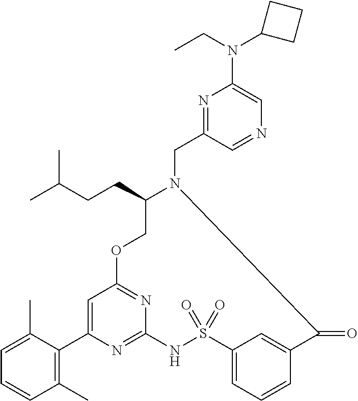 [SMILES: CCN(c1cncc(CN2C(=O)c3cccc(c3)S(=O)(=O)Nc3nc(cc(-c4c(C)cccc4C)n3)OC[C@H]2CCC(C)C)n1)C1CCC1] | 1.73 | 683.325 | 684.5 | A |
| 1765 | 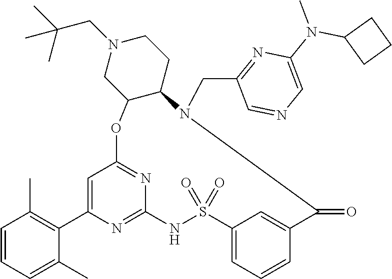 [SMILES: Cc1cccc(C)c1-c1cc2nc(n1)NS(=O)(=O)c1cccc(c1)C(=O)N(Cc1cncc(N(C)C3CCC3)n1)[C@@H]1CCN(CC(C)(C)C)CC1O2] | 1.28 | 724.352 | 725.3 | A |
| 1766 | 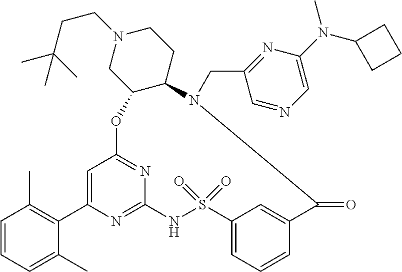 [SMILES: Cc1cccc(C)c1-c1cc2nc(n1)NS(=O)(=O)c1cccc(c1)C(=O)N(Cc1cncc(N(C)C3CCC3)n1)[C@@H]1CCN(CCC(C)(C)C)C[C@H]1O2] | 1.34 | 738.368 | 739.3 | A |
| 1767 | 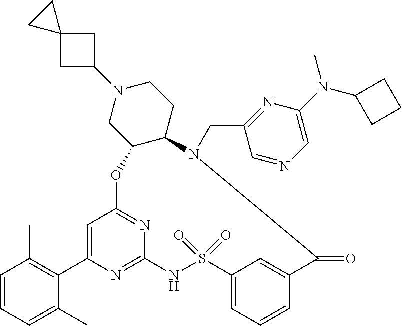 [SMILES: Cc1cccc(C)c1-c1cc2nc(n1)NS(=O)(=O)c1cccc(c1)C(=O)N(Cc1cncc(N(C)C3CCC3)n1)[C@@H]1CCN(C3CC4(CC4)C3)C[C@H]1O2] | 1.25 | 734.336 | 735.3 | A |
| 1768 | 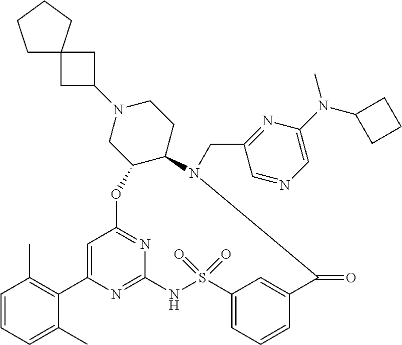 [SMILES: Cc1cccc(C)c1-c1cc2nc(n1)NS(=O)(=O)c1cccc(c1)C(=O)N(Cc1cncc(N(C)C3CCC3)n1)[C@@H]1CCN(C3CC4(CCCC4)C3)C[C@H]1O2] | 1.39 | 762.368 | 763.3 | A |
| 1769 | 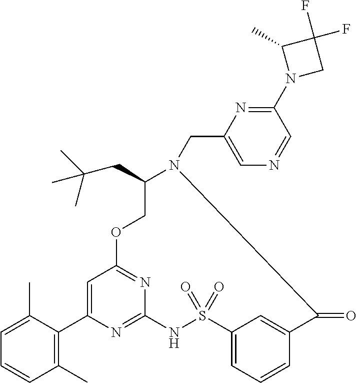 [SMILES: Cc1cccc(C)c1-c1cc2nc(n1)NS(=O)(=O)c1cccc(c1)C(=O)N(Cc1cncc(N3CC(F)(F)[C@H]3C)n1)[C@H](CC(C)(C)C)CO2] | 1.92 | 691.275 | 692.8 | A |
| 1770 | 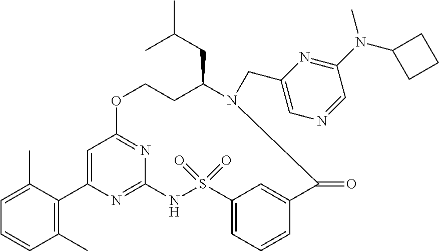 [SMILES: Cc1cccc(C)c1-c1cc2nc(n1)NS(=O)(=O)c1cccc(c1)C(=O)N(Cc1cncc(N(C)C3CCC3)n1)[C@H](CC(C)C)CCO2] | 1.86 | 669.31 | 670.86 | A |
| 1771 | 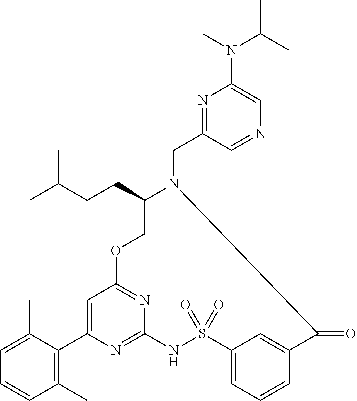 [SMILES: Cc1cccc(C)c1-c1cc2nc(n1)NS(=O)(=O)c1cccc(c1)C(=O)N(Cc1cncc(N(C)C(C)C)n1)[C@H](CCC(C)C)CO2] | 1.56 | 657.31 | 658.5 | A |
| 1772 | 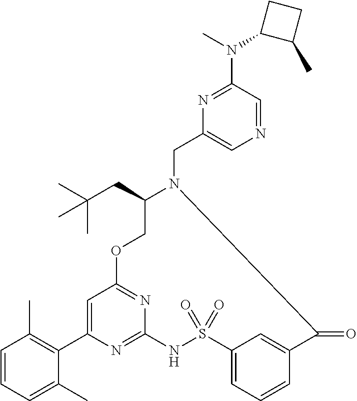 [SMILES: Cc1cccc(C)c1-c1cc2nc(n1)NS(=O)(=O)c1cccc(c1)C(=O)N(Cc1cncc(N(C)[C@@H]3CC[C@H]3C)n1)[C@H](CC(C)(C)C)CO2] | 1.68 | 683.325 | 684.5 | A |
| 1773 | 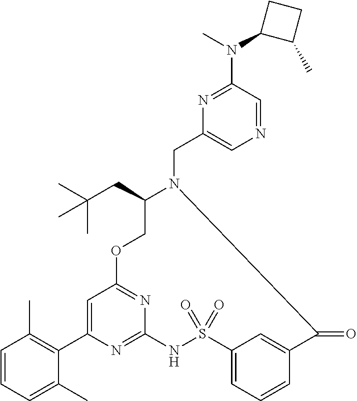 [SMILES: Cc1cccc(C)c1-c1cc2nc(n1)NS(=O)(=O)c1cccc(c1)C(=O)N(Cc1cncc(N(C)[C@H]3CC[C@@H]3C)n1)[C@H](CC(C)(C)C)CO2] | 1.68 | 683.325 | 684.4 | A |
| 1774 | 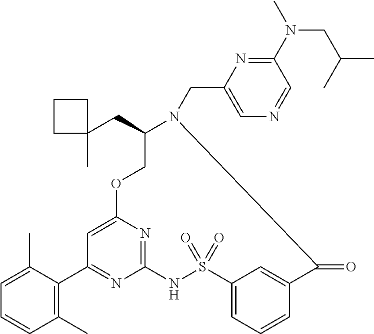 [SMILES: Cc1cccc(C)c1-c1cc2nc(n1)NS(=O)(=O)c1cccc(c1)C(=O)N(Cc1cncc(N(C)CC(C)C)n1)[C@H](CC1(C)CCC1)CO2] | 1.89 | 683.325 | 684.3 | A |
| 1775 | 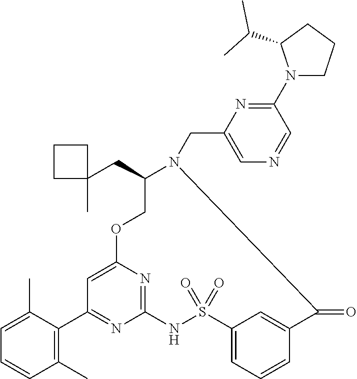 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](CC1(C)CCC1)CO2] | 1.96 | 709.341 | 710.3 | A |
| 1776 | 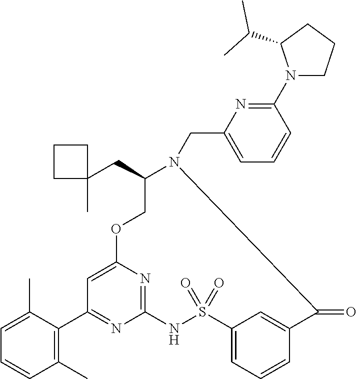 [SMILES: Cc1cccc(C)c1-c1cc2nc(n1)NS(=O)(=O)c1cccc(c1)C(=O)N(Cc1cccc(N3CCC[C@H]3C(C)C)n1)[C@H](CC1(C)CCC1)CO2] | 1.59 | 708.346 | 709.3 | A |
| 1777 | 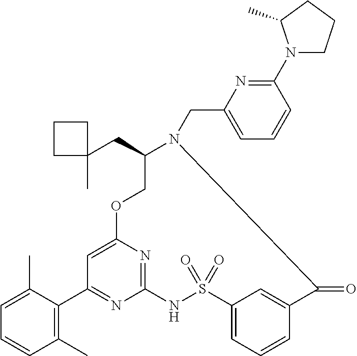 [SMILES: Cc1cccc(C)c1-c1cc2nc(n1)NS(=O)(=O)c1cccc(c1)C(=O)N(Cc1cccc(N3CCC[C@H]3C)n1)[C@H](CC1(C)CCC1)CO2] | 1.45 | 680.314 | 681.3 | A |
| 1778 | 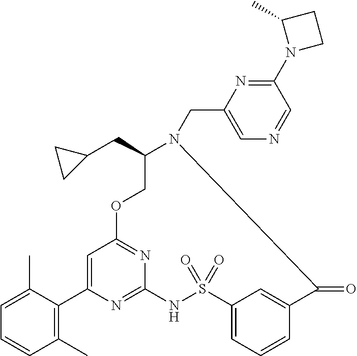 [SMILES: Cc1cccc(C)c1-c1cc2nc(n1)NS(=O)(=O)c1cccc(c1)C(=O)N(Cc1cncc(N3CC[C@H]3C)n1)[C@H](CC1CC1)CO2] | 1.63 | 639.263 | 640.2 | A |
| 1779 | 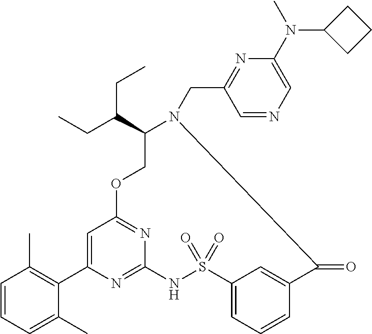 [SMILES: CCC(CC)[C@@H]1COc2cc(-c3c(C)cccc3C)nc(n2)NS(=O)(=O)c2cccc(c2)C(=O)N1Cc1cncc(N(C)C2CCC2)n1] | 1.91 | 669.31 | 670.9 | A |
| 1780 | 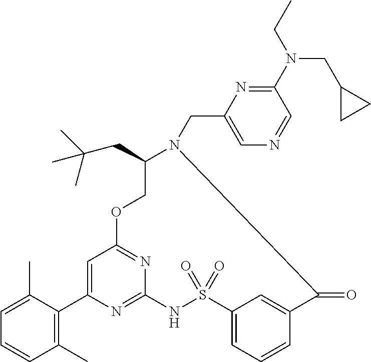 [SMILES: CCN(CC1CC1)c1cncc(CN2C(=O)c3cccc(c3)S(=O)(=O)Nc3nc(cc(-c4c(C)cccc4C)n3)OC[C@H]2CC(C)(C)C)n1] | 1.9 | 683.325 | 684.8 | A |
| 1781 | 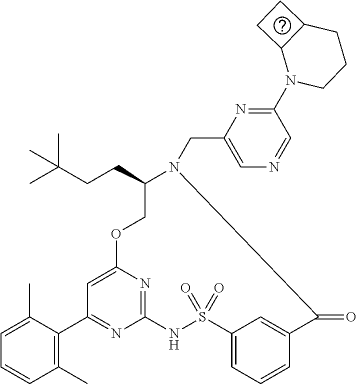 [SMILES: Cc1cccc(C)c1-c1cc2nc(n1)NS(=O)(=O)c1cccc(c1)C(=O)N(Cc1cncc(N3CCCC4CCC43)n1)[C@H](CCC(C)(C)C)CO2] | 2.09 | 709.341 | 710.8 | A |
| 1782 | 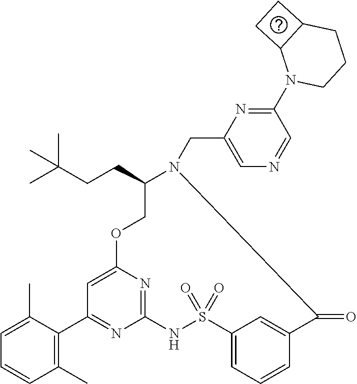 [SMILES: Cc1cccc(C)c1-c1cc2nc(n1)NS(=O)(=O)c1cccc(c1)C(=O)N(Cc1cncc(N3CCCC4CCC43)n1)[C@H](CCC(C)(C)C)CO2] | 2.1 | 709.341 | 710.9 | A |
| 1783 | 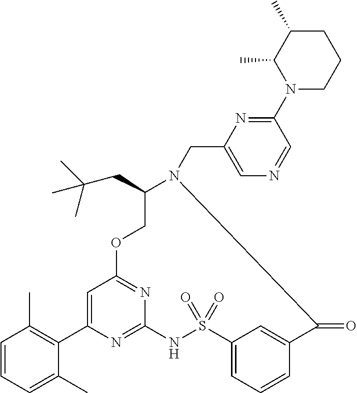 [SMILES: Cc1cccc(C)c1-c1cc2nc(n1)NS(=O)(=O)c1cccc(c1)C(=O)N(Cc1cncc(N3CCC[C@@H](C)[C@H]3C)n1)[C@H](CC(C)(C)C)CO2] | 2 | 697.341 | 698.8 | A |
| 1784 | 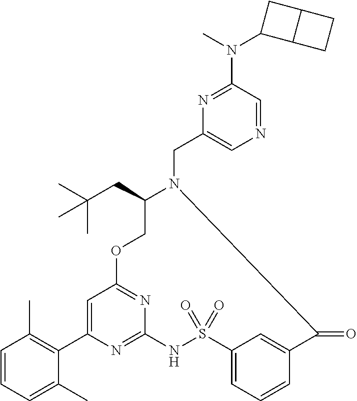 [SMILES: Cc1cccc(C)c1-c1cc2nc(n1)NS(=O)(=O)c1cccc(c1)C(=O)N(Cc1cncc(N(C)C3CC4CCC43)n1)[C@H](CC(C)(C)C)CO2] | 1.75 | 695.325 | 696.6 | A |
| 1785 | 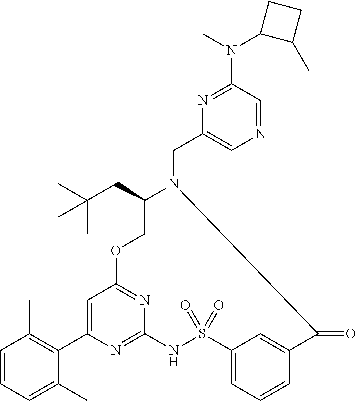 [SMILES: Cc1cccc(C)c1-c1cc2nc(n1)NS(=O)(=O)c1cccc(c1)C(=O)N(Cc1cncc(N(C)C3CCC3C)n1)[C@H](CC(C)(C)C)CO2] | 1.7 | 683.325 | 684.5 | A |
| 1786 | 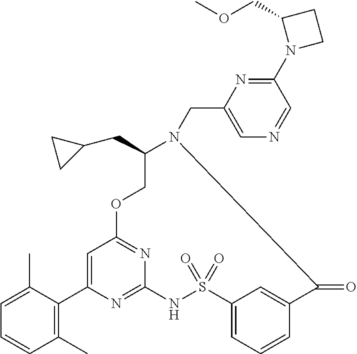 [SMILES: COC[C@@H]1CCN1c1cncc(CN2C(=O)c3cccc(c3)S(=O)(=O)Nc3nc(cc(-c4c(C)cccc4C)n3)OC[C@H]2CC2CC2)n1] | 1.57 | 669.273 | 670.2 | A |
| 1787 | 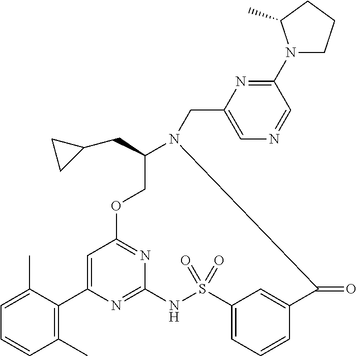 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CC1)CO2] | 1.65 | 653.278 | 654.45 | A |
| 1788 | 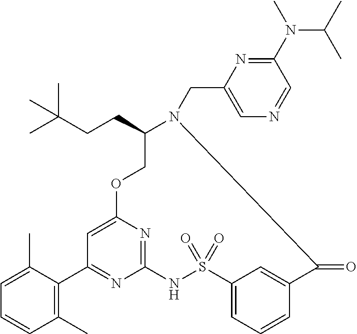 [SMILES: Cc1cccc(C)c1-c1cc2nc(n1)NS(=O)(=O)c1cccc(c1)C(=O)N(Cc1cncc(N(C)C(C)C)n1)[C@H](CCC(C)(C)C)CO2] | 182 | 671.325 | 672.3 | A |
| 1789 | 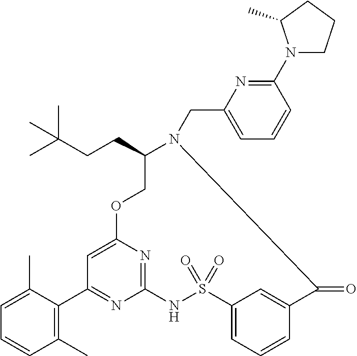 [SMILES: Cc1cccc(C)c1-c1cc2nc(n1)NS(=O)(=O)c1cccc(c1)C(=O)N(Cc1cccc(N3CCC[C@H]3C)n1)[C@H](CCC(C)(C)C)CO2] | 1.51 | 682.33 | 683.3 | A |
| 1790 | 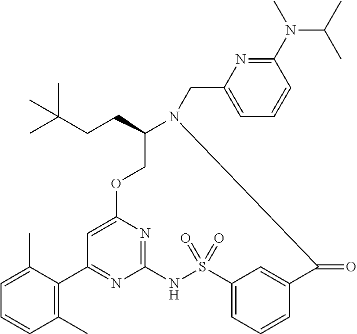 [SMILES: Cc1cccc(C)c1-c1cc2nc(n1)NS(=O)(=O)c1cccc(c1)C(=O)N(Cc1cccc(N(C)C(C)C)n1)[C@H](CCC(C)(C)C)CO2] | 150 | 670.33 | 671.3 | A |
| 1791 | 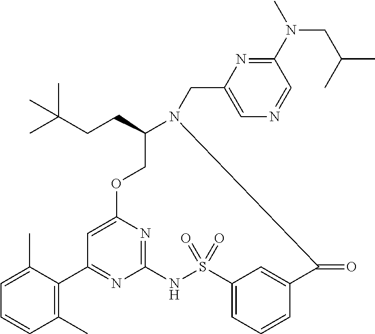 [SMILES: Cc1cccc(C)c1-c1cc2nc(n1)NS(=O)(=O)c1cccc(c1)C(=O)N(Cc1cncc(N(C)CC(C)C)n1)[C@H](CCC(C)(C)C)CO2] | 1.93 | 685.341 | 686.3 | A |
| 1792 | 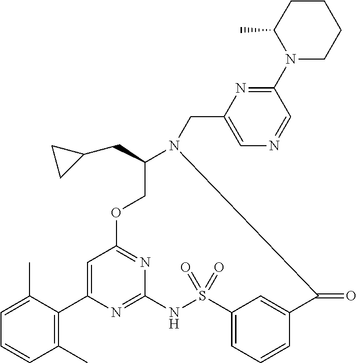 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CC1)CO2] | 1.71 | 667.294 | 668.1 | A |
| 1793 | 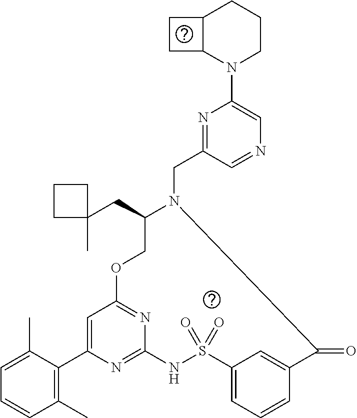 [SMILES: Cc1cccc(C)c1-c1cc2nc(n1)NS(=O)(=O)c1cccc(c1)C(=O)N(Cc1cncc(N3CCCC4CCC43)n1)[C@H](CC1(C)CCC1)CO2] | 1.73 | 707.325 | 708.6 | A |
| 1794 | 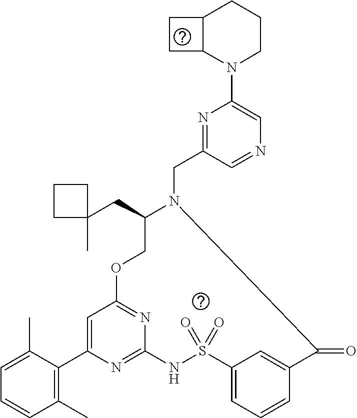 [SMILES: Cc1cccc(C)c1-c1cc2nc(n1)NS(=O)(=O)c1cccc(c1)C(=O)N(Cc1cncc(N3CCCC4CCC43)n1)[C@H](CC1(C)CCC1)CO2] | 1.74 | 707.325 | 708.5 | A |
| 1795 | 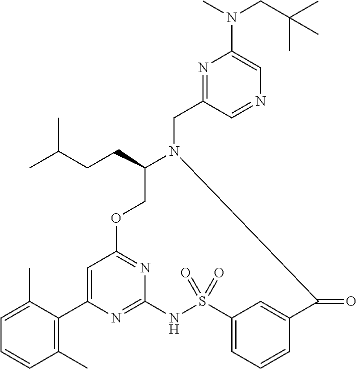 [SMILES: Cc1cccc(C)c1-c1cc2nc(n1)NS(=O)(=O)c1cccc(c1)C(=O)N(Cc1cncc(N(C)CC(C)(C)C)n1)[C@H](CCC(C)C)CO2] | 1.76 | 685.341 | 686.6 | A |
| 1796 | 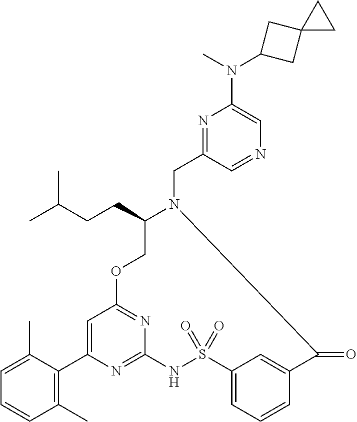 [SMILES: Cc1cccc(C)c1-c1cc2nc(n1)NS(=O)(=O)c1cccc(c1)C(=O)N(Cc1cncc(N(C)C3CC4(CC4)C3)n1)[C@H](CCC(C)C)CO2] | 1.78 | 695.325 | 696.5 | A |
| 1797 | 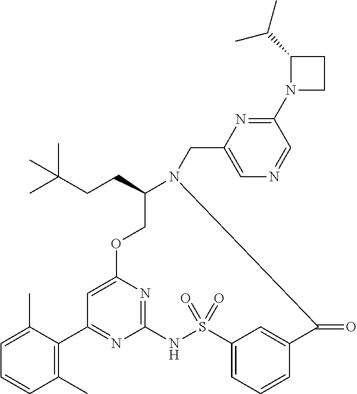 [SMILES: Cc1cccc(C)c1-c1cc2nc(n1)NS(=O)(=O)c1cccc(c1)C(=O)N(Cc1cncc(N3CC[C@H]3C(C)C)n1)[C@H](CCC(C)(C)C)CO2] | 2.11 | 697.341 | 698.9 | A |
| 1798 | 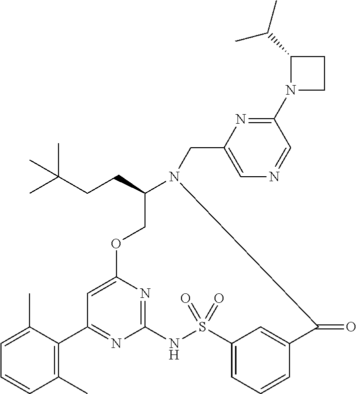 [SMILES: Cc1cccc(C)c1-c1cc2nc(n1)NS(=O)(=O)c1cccc(c1)C(=O)N(Cc1cncc(N3CC[C@H]3C(C)C)n1)[C@H](CCC(C)(C)C)CO2] | 2.12 | 697.341 | 698.8 | A |
| 1799 | 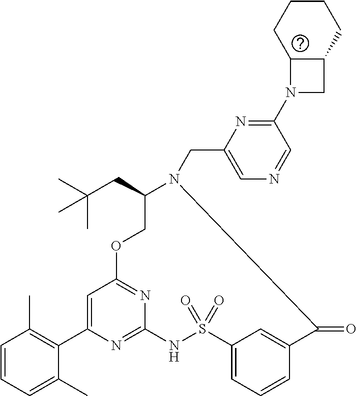 | 2 | 695.325 | 696.8 | A |
| 1800 | 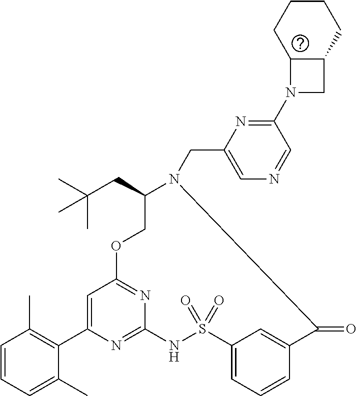 | 2.01 | 695.325 | 696.8 | A |
| 1801 | 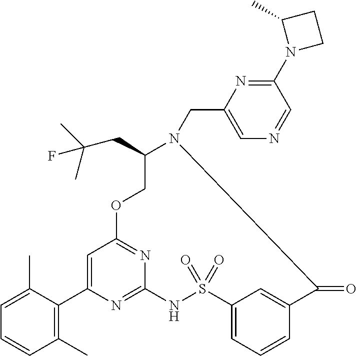 [SMILES: Cc1cccc(C)c1-c1cc2nc(n1)NS(=O)(=O)c1cccc(c1)C(=O)N(Cc1cncc(N3CC[C@H]3C)n1)[C@H](CC(C)(C)F)CO2] | 1.567 | 659.269 | 660.2 | A |
| 1802 | 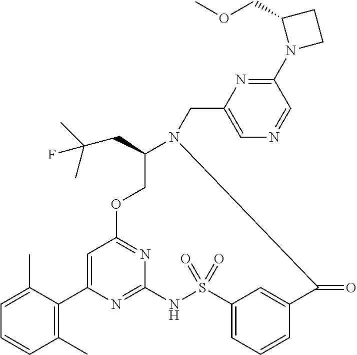 [SMILES: COC[C@@H]1CCN1c1cncc(CN2C(=O)c3cccc(c3)S(=O)(=O)Nc3nc(cc(-c4c(C)cccc4C)n3)OC[C@H]2CC(C)(C)F)n1] | 1.59 | 689.28 | 690 | A |
| 1803 | 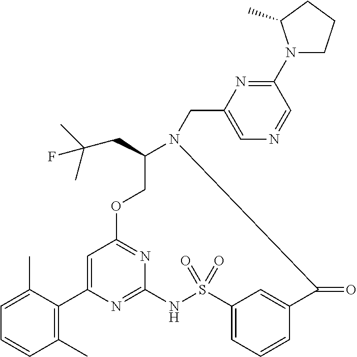 [SMILES: Cc1cccc(C)c1-c1cc2nc(n1)NS(=O)(=O)c1cccc(c1)C(=O)N(Cc1cncc(N3CCC[C@H]3C)n1)[C@H](CC(C)(C)F)CO2] | 1.57 | 673.285 | 674.2 | A |
| 1804 | 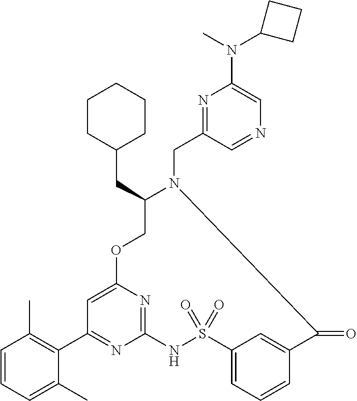 [SMILES: Cc1cccc(C)c1-c1cc2nc(n1)NS(=O)(=O)c1cccc(c1)C(=O)N(Cc1cncc(N(C)C3CCC3)n1)[C@H](CC1CCCCC1)CO2] | 2.04 | 695.325 | 696.8 | A |
| 1805 | 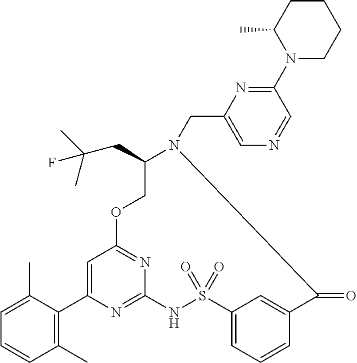 [SMILES: Cc1cccc(C)c1-c1cc2nc(n1)NS(=O)(=O)c1cccc(c1)C(=O)N(Cc1cncc(N3CCCC[C@H]3C)n1)[C@H](CC(C)(C)F)CO2] | 1.65 | 687.3 | 688.2 | A |
| 1324 | 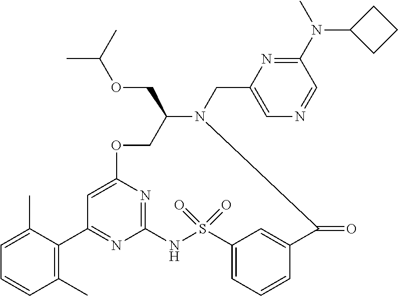 [SMILES: Cc1cccc(C)c1-c1cc2nc(n1)NS(=O)(=O)c1cccc(c1)C(=O)N(Cc1cncc(N(C)C3CCC3)n1)[C@H](COC(C)C)CO2] | 4.43 | 671.289 | 672.2 | Y |
| 1806 | 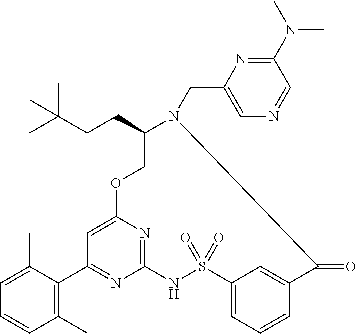 [SMILES: Cc1cccc(C)c1-c1cc2nc(n1)NS(=O)(=O)c1cccc(c1)C(=O)N(Cc1cncc(N(C)C)n1)[C@H](CCC(C)(C)C)CO2] | 1.72 | 643.294 | 644.2 | A |
| 1807 | 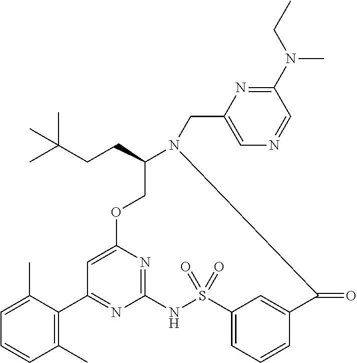 [SMILES: CCN(C)c1cncc(CN2C(=O)c3cccc(c3)S(=O)(=O)Nc3nc(cc(-c4c(C)cccc4C)n3)OC[C@H]2CCC(C)(C)C)n1] | 1.77 | 657.31 | 658.2 | A |
| 1808 | 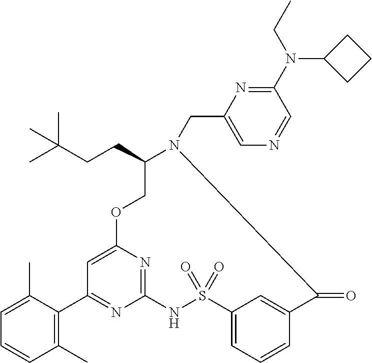 [SMILES: CCN(c1cncc(CN2C(=O)c3cccc(c3)S(=O)(=O)Nc3nc(cc(-c4c(C)cccc4C)n3)OC[C@H]2CCC(C)(C)C)n1)C1CCC1] | 1.96 | 697.341 | 698.3 | A |
| 1809 | 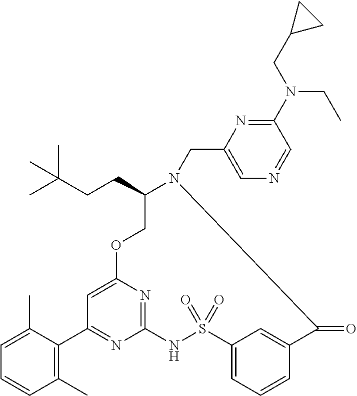 [SMILES: CCN(CC1CC1)c1cncc(CN2C(=O)c3cccc(c3)S(=O)(=O)Nc3nc(cc(-c4c(C)cccc4C)n3)OC[C@H]2CCC(C)(C)C)n1] | 1.92 | 697.341 | 698.3 | A |
| 1810 | 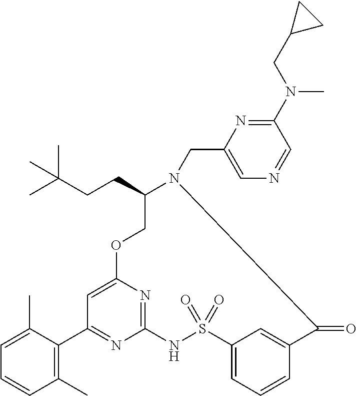 [SMILES: Cc1cccc(C)c1-c1cc2nc(n1)NS(=O)(=O)c1cccc(c1)C(=O)N(Cc1cncc(N(C)CC3CC3)n1)[C@H](CCC(C)(C)C)CO2] | 1.87 | 683.325 | 684.3 | A |
| 1811 | 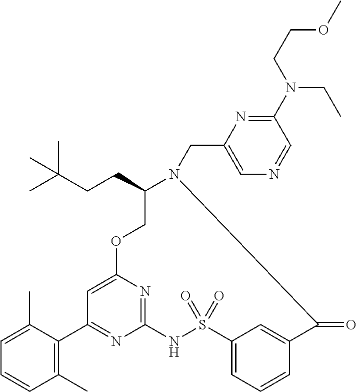 [SMILES: CCN(CCOC)c1cncc(CN2C(=O)c3cccc(c3)S(=O)(=O)Nc3nc(cc(-c4c(C)cccc4C)n3)OC[C@H]2CCC(C)(C)C)n1] | 1.79 | 701.336 | 702.3 | A |
| 1812 | 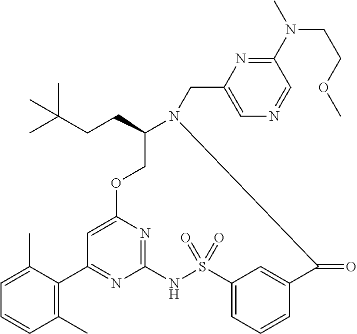 [SMILES: COCCN(C)c1cncc(CN2C(=O)c3cccc(c3)S(=O)(=O)Nc3nc(cc(-c4c(C)cccc4C)n3)OC[C@H]2CCC(C)(C)C)n1] | 1.74 | 687.32 | 688.2 | A |
| 1813 | 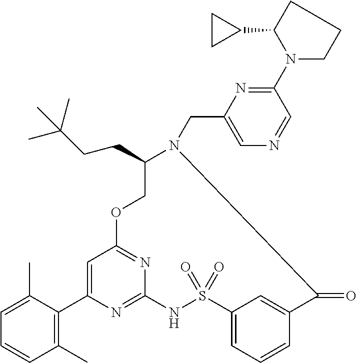 [SMILES: Cc1cccc(C)c1-c1cc2nc(n1)NS(=O)(=O)c1cccc(c1)C(=O)N(Cc1cncc(N3CCC[C@H]3C3CC3)n1)[C@H](CCC(C)(C)C)CO2] | 1.95 | 709.341 | 710.3 | A |
| 1814 | 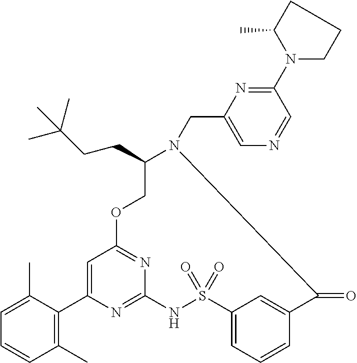 [SMILES: Cc1cccc(C)c1-c1cc2nc(n1)NS(=O)(=O)c1cccc(c1)C(=O)N(Cc1cncc(N3CCC[C@H]3C)n1)[C@H](CCC(C)(C)C)CO2] | 1.85 | 683.325 | 684.2 | A |
| 1815 | 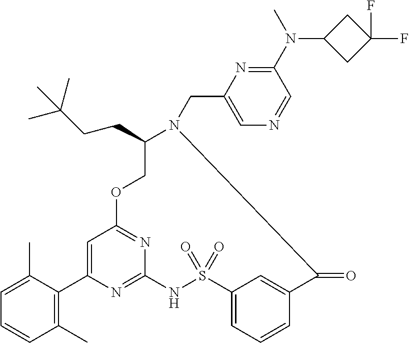 [SMILES: Cc1cccc(C)c1-c1cc2nc(n1)NS(=O)(=O)c1cccc(c1)C(=O)N(Cc1cncc(N(C)C3CC(F)(F)C3)n1)[C@H](CCC(C)(C)C)CO2] | 1.91 | 719.307 | 720.2 | A |
| 1816 | 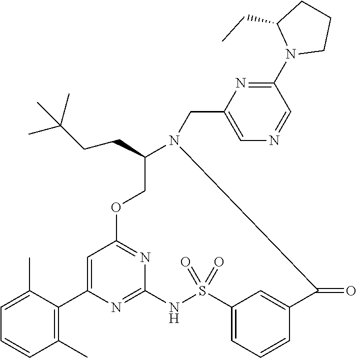 [SMILES: CC[C@@H]1CCCN1c1cncc(CN2C(=O)c3cccc(c3)S(=O)(=O)Nc3nc(cc(-c4c(C)cccc4C)n3)OC[C@H]2CCC(C)(C)C)n1] | 1.93 | 697.341 | 698.3 | A |
| 1817 | 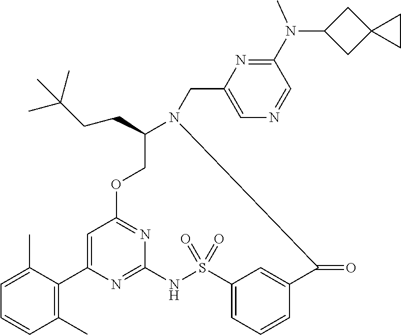 [SMILES: Cc1cccc(C)c1-c1cc2nc(n1)NS(=O)(=O)c1cccc(c1)C(=O)N(Cc1cncc(N(C)C3CC4(CC4)C3)n1)[C@H](CCC(C)(C)C)CO2] | 2.03 | 709.341 | 710.3 | A |
| 1818 | 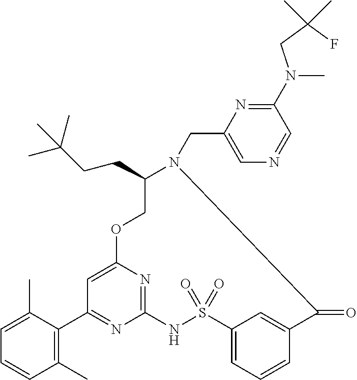 [SMILES: Cc1cccc(C)c1-c1cc2nc(n1)NS(=O)(=O)c1cccc(c1)C(=O)N(Cc1cncc(N(C)CC(C)(C)F)n1)[C@H](CCC(C)(C)C)CO2] | 1.88 | 703.332 | 704.3 | A |
| 1819 | 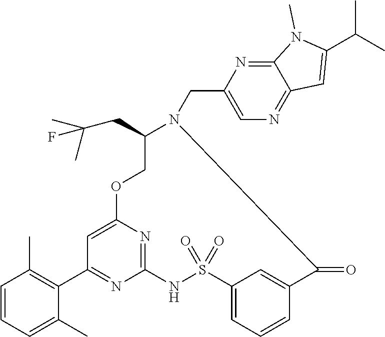 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)F)CO2] | 1.58 | 685.285 | 686.2 | A |
| 1820 | 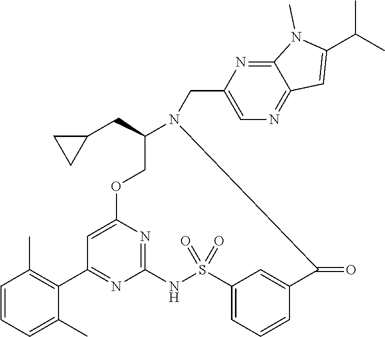 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CC1)CO2] | 1.63 | 665.278 | 666.2 | A |
| 1821 | 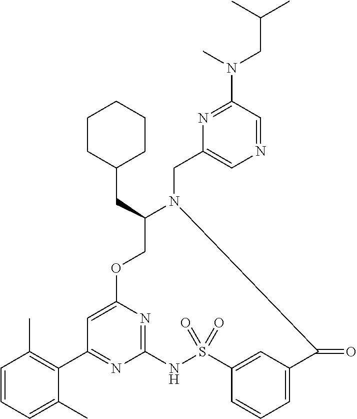 [SMILES: Cc1cccc(C)c1-c1cc2nc(n1)NS(=O)(=O)c1cccc(c1)C(=O)N(Cc1cncc(N(C)CC(C)C)n1)[C@H](CC1CCCCC1)CO2] | 2.03 | 697.341 | 698.8 | A |
| 1822 | 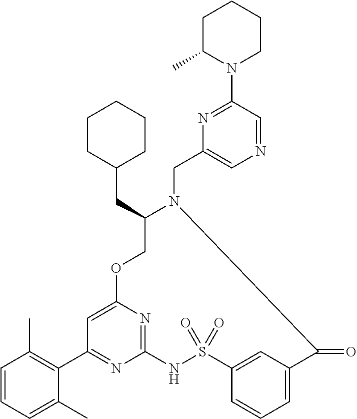 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CCCCC1)CO2] | 2.02 | 709.341 | 710.9 | A |
| 1823 | 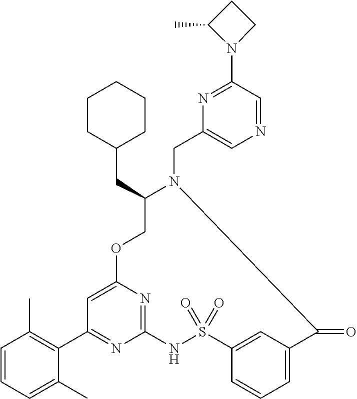 [SMILES: Cc1cccc(C)c1-c1cc2nc(n1)NS(=O)(=O)c1cccc(c1)C(=O)N(Cc1cncc(N3CC[C@H]3C)n1)[C@H](CC1CCCCC1)CO2] | 1.95 | 681.31 | 682.8 | A |
| 1824 | 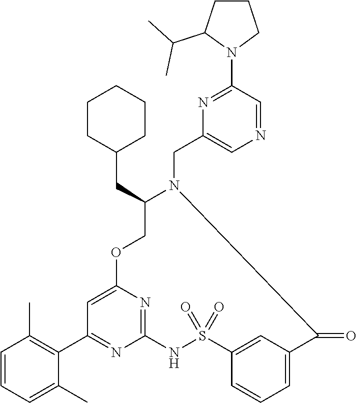 [SMILES: Cc1cccc(C)c1-c1cc2nc(n1)NS(=O)(=O)c1cccc(c1)C(=O)N(Cc1cncc(N3CCCC3C(C)C)n1)[C@H](CC1CCCCC1)CO2] | 2.1 | 723.357 | 724.8 | A |
| 1825 | 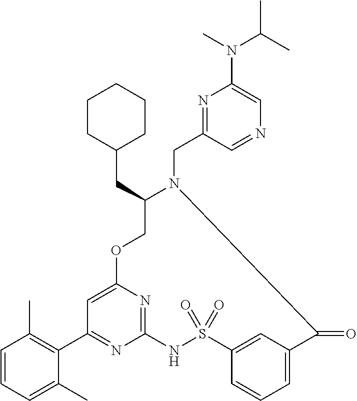 [SMILES: Cc1cccc(C)c1-c1cc2nc(n1)NS(=O)(=O)c1cccc(c1)C(=O)N(Cc1cncc(N(C)C(C)C)n1)[C@H](CC1CCCCC1)CO2] | 1.91 | 683.325 | 684.8 | A |
| 1826 | 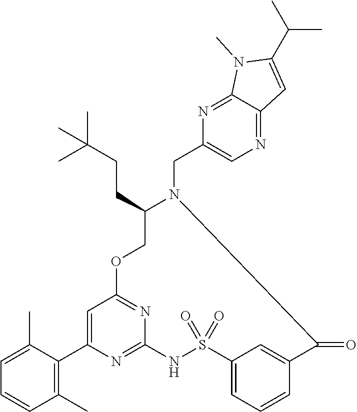 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC(C)(C)C)CO2] | 1.91 | 695.325 | 696.8 | A |
| 1827 | 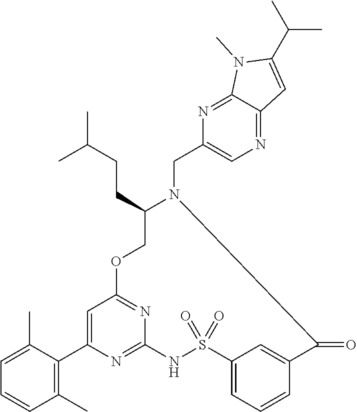 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC(C)C)CO2] | 1.84 | 681.31 | 682.8 | A |
| 1828 | 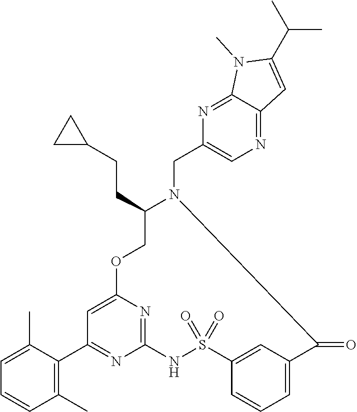 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC1CC1)CO2] | 1.77 | 679.294 | 680.8 | A |
| 1829 | 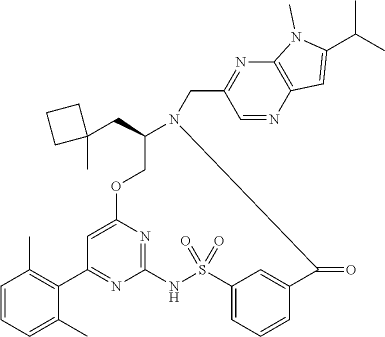 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(C)CCC1)CO2] | 1.78 | 693.31 | 694.3 | A |
| 1830 | 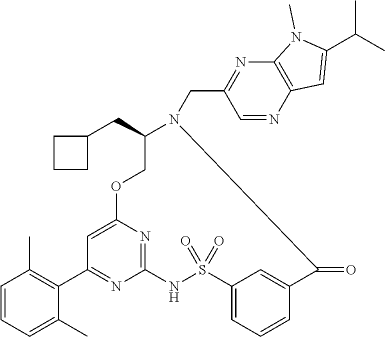 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCC1)CO2] | 1.72 | 679.294 | 680.2 | A |
| 1831 | 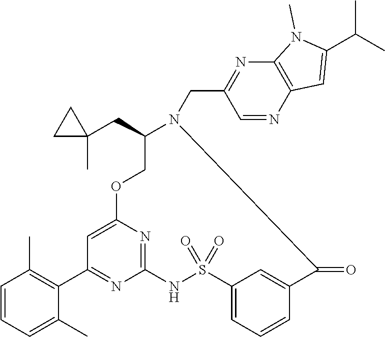 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(C)CC1)CO2] | 1.68 | 679.294 | 680.2 | A |
| 1832 | 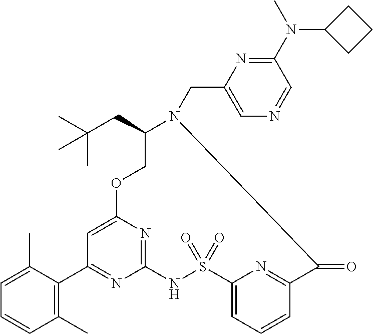 [SMILES: Cc1cccc(C)c1-c1cc2nc(n1)NS(=O)(=O)c1cccc(n1)C(=O)N(Cc1cncc(N(C)C3CCC3)n1)[C@H](CC(C)(C)C)CO2] | 1.75 | 670.305 | 671.3 | A |
| 1833 | 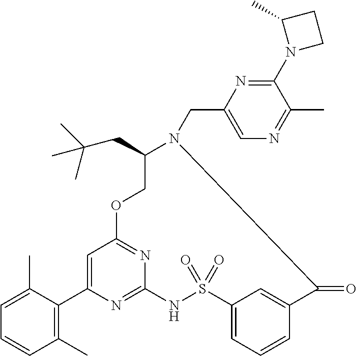 [SMILES: Cc1cccc(C)c1-c1cc2nc(n1)NS(=O)(=O)c1cccc(c1)C(=O)N(Cc1cnc(C)c(N3CC[C@H]3C)n1)[C@H](CC(C)(C)C)CO2] | 1.66 | 669.31 | 670.3 | A |
| 1834 | 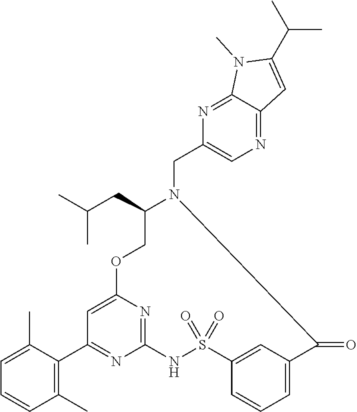 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)C)CO2] | 1.51 | 667.294 | 668.5 | A |
| 1835 | 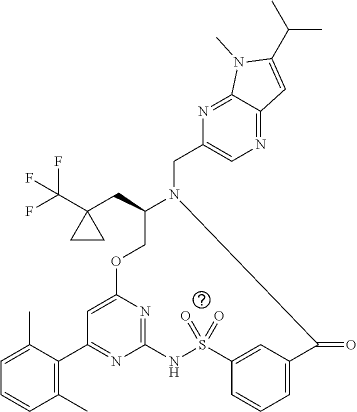 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(C(F)(F)F)CC1)CO2] | 1.48 | 733.266 | 734.5 | A |
| 1836 | 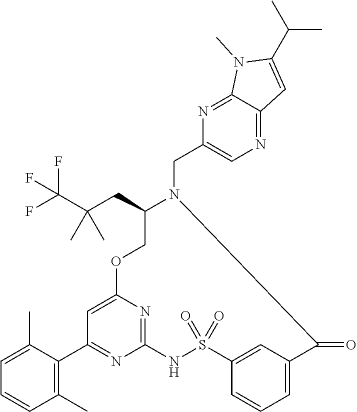 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C(F)(F)F)CO2] | 1.54 | 735.281 | 736.5 | A |
| 1325 | 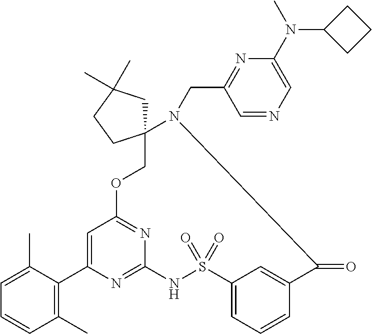 [SMILES: Cc1cccc(C)c1-c1cc2nc(n1)NS(=O)(=O)c1cccc(c1)C(=O)N(Cc1cncc(N(C)C3CCC3)n1)[C@]1(CCC(C)(C)C1)CO2] | 1.9 | 681.31 | 682.8 | A |
| 1326 | 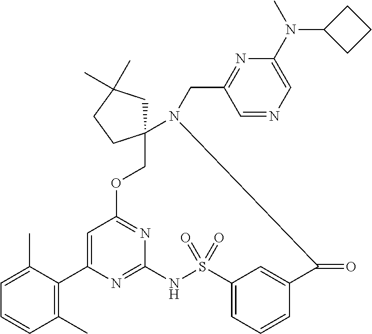 [SMILES: Cc1cccc(C)c1-c1cc2nc(n1)NS(=O)(=O)c1cccc(c1)C(=O)N(Cc1cncc(N(C)C3CCC3)n1)[C@]1(CCC(C)(C)C1)CO2] | 1.9 | 681.31 | 682.8 | A |
| 1837 | 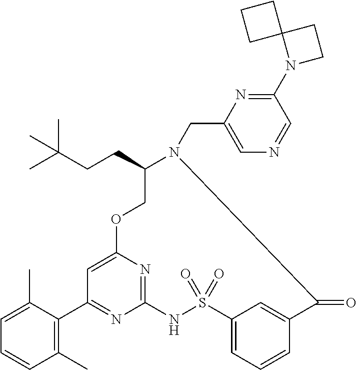 [SMILES: Cc1cccc(C)c1-c1cc2nc(n1)NS(=O)(=O)c1cccc(c1)C(=O)N(Cc1cncc(N3CCC34CCC4)n1)[C@H](CCC(C)(C)C)CO2] | 1.977 | 695.325 | 696.3 | A |
| 1838 | 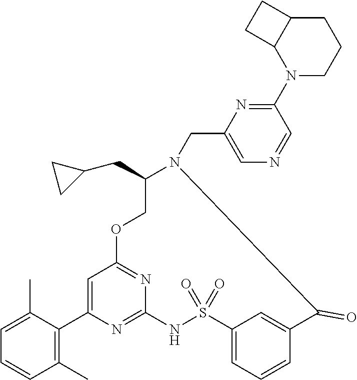 [SMILES: Cc1cccc(C)c1-c1cc2nc(n1)NS(=O)(=O)c1cccc(c1)C(=O)N(Cc1cncc(N3CCCC4CCC43)n1)[C@H](CC1CC1)CO2] | 1.78 | 679.294 | 680.2 | A |
| 1839 | 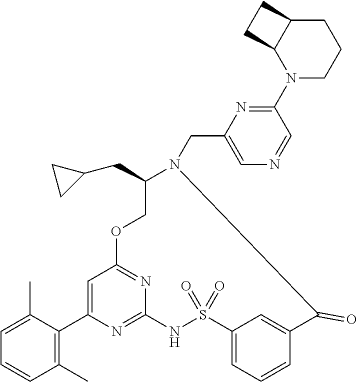 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CC1CC1)CO2] | 1.79 | 679.294 | 680.2 | A |
| 1840 | 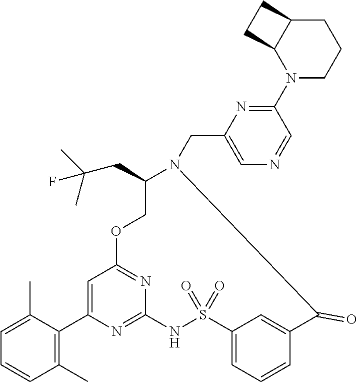 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CC(C)(C)F)CO2] | 1.72 | 699.3 | 700.2 | A |
| 1841 | 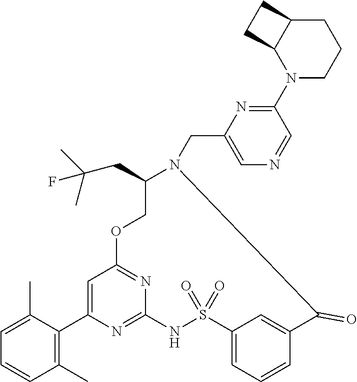 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CC(C)(C)F)CO2] | 1.73 | 699.3 | 700.3 | A |
| 1842 | 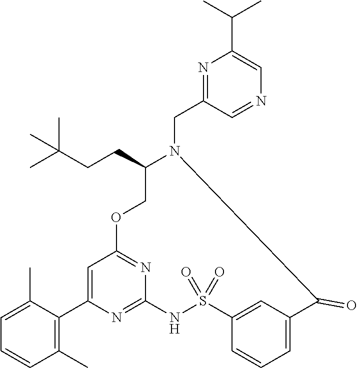 [SMILES: Cc1cccc(C)c1-c1cc2nc(n1)NS(=O)(=O)c1cccc(c1)C(=O)N(Cc1cncc(C(C)C)n1)[C@H](CCC(C)(C)C)CO2] | 1.98 | 642.299 | 643.2 | A |
| 1843 | 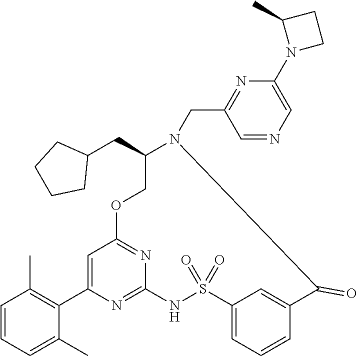 [SMILES: Cc1cccc(C)c1-c1cc2nc(n1)NS(=O)(=O)c1cccc(c1)C(=O)N(Cc1cncc(N3CC[C@@H]3C)n1)[C@H](CC1CCCC1)CO2] | 1.85 | 667.294 | 668.2 | A |
| 1844 | 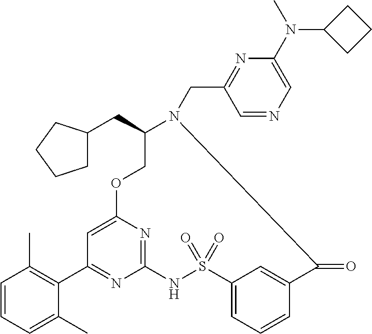 [SMILES: Cc1cccc(C)c1-c1cc2nc(n1)NS(=O)(=O)c1cccc(c1)C(=O)N(Cc1cncc(N(C)C3CCC3)n1)[C@H](CC1CCCC1)CO2] | 1.88 | 681.31 | 682.3 | A |
| 1845 | 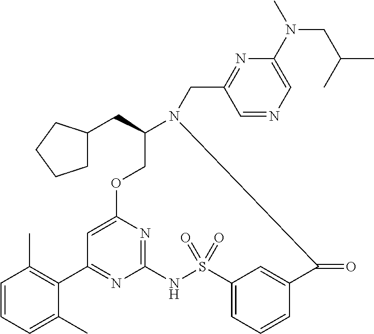 [SMILES: Cc1cccc(C)c1-c1cc2nc(n1)NS(=O)(=O)c1cccc(c1)C(=O)N(Cc1cncc(N(C)CC(C)C)n1)[C@H](CC1CCCC1)CO2] | 1.9 | 683.325 | 684.3 | A |
| 1846 | 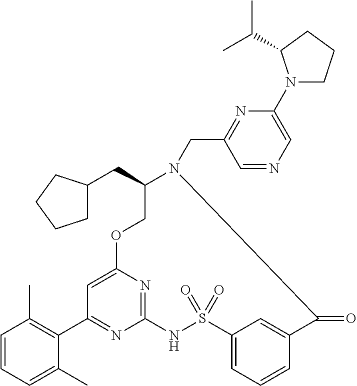 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](CC1CCCC1)CO2] | 1.97 | 709.341 | 710.3 | A |
| 1847 | 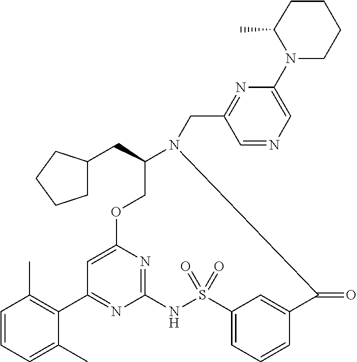 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CCCC1)CO2] | 1.89 | 695.325 | 696.3 | A |
| 1848 | 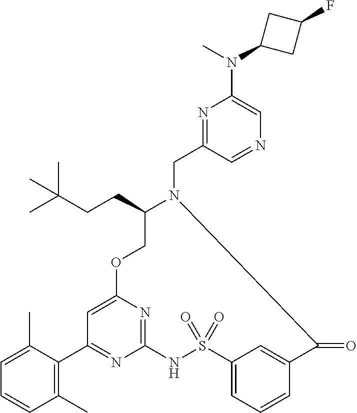 [SMILES: Cc1cccc(C)c1-c1cc2nc(n1)NS(=O)(=O)c1cccc(c1)C(=O)N(Cc1cncc(N(C)[C@H]3C[C@@H](F)C3)n1)[C@H](CCC(C)(C)C)CO2] | 1.67 | 701.316 | 702.5 | A |
| 1849 | 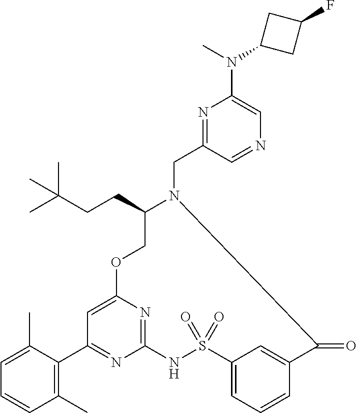 [SMILES: Cc1cccc(C)c1-c1cc2nc(n1)NS(=O)(=O)c1cccc(c1)C(=O)N(Cc1cncc(N(C)[C@H]3C[C@H](F)C3)n1)[C@H](CCC(C)(C)C)CO2] | 1.67 | 701.316 | 702.5 | A |
| 1850 | 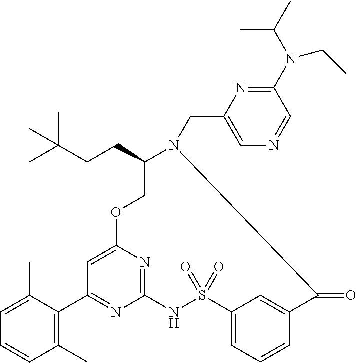 [SMILES: CCN(c1cncc(CN2C(=O)c3cccc(c3)S(=O)(=O)Nc3nc(cc(-c4c(C)cccc4C)n3)OC[C@H]2CCC(C)(C)C)n1)C(C)C] | 1.93 | 685.341 | 686.8 | A |
| 1851 | 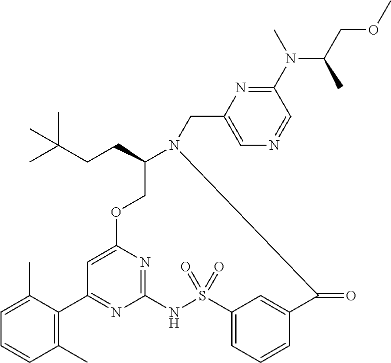 [SMILES: COC[C@@H](C)N(C)c1cncc(CN2C(=O)c3cccc(c3)S(=O)(=O)Nc3nc(cc(-c4c(C)cccc4C)n3)OC[C@H]2CCC(C)(C)C)n1] | 1.82 | 701.336 | 702.8 | A |
| 1852 | 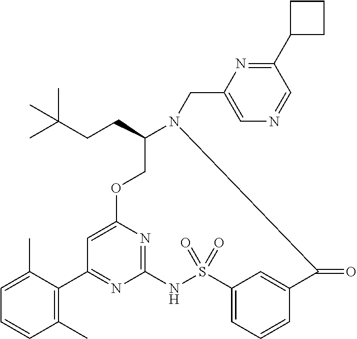 [SMILES: Cc1cccc(C)c1-c1cc2nc(n1)NS(=O)(=O)c1cccc(c1)C(=O)N(Cc1cncc(C3CCC3)n1)[C@H](CCC(C)(C)C)CO2] | 2.01 | 654.299 | 655.2 | A |
| 1327 | 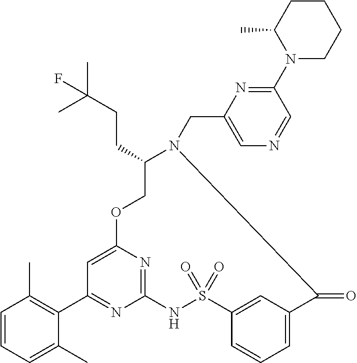 [SMILES: Cc1cccc(C)c1-c1cc2nc(n1)NS(=O)(=O)c1cccc(c1)C(=O)N(Cc1cncc(N3CCCC[C@H]3C)n1)[C@@H](CCC(C)(C)F)CO2] | 1.77 | 701.316 | 702.8 | A |
| 1328 | 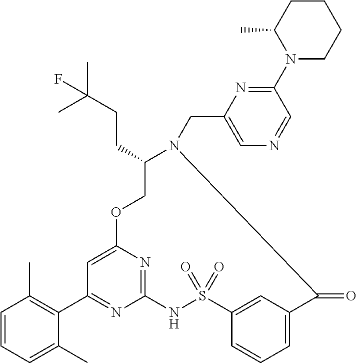 [SMILES: Cc1cccc(C)c1-c1cc2nc(n1)NS(=O)(=O)c1cccc(c1)C(=O)N(Cc1cncc(N3CCCC[C@H]3C)n1)[C@@H](CCC(C)(C)F)CO2] | 1.77 | 701.316 | 702.8 | A |
| 1853 | 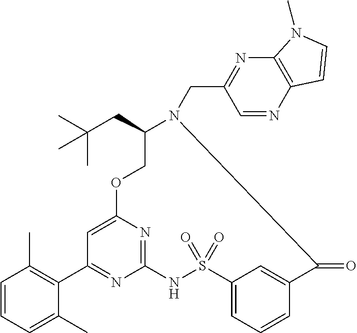 [SMILES: Cc1cccc(C)c1-c1cc2nc(n1)NS(=O)(=O)c1cccc(c1)C(=O)N(Cc1cnc3ccn(C)c3n1)[C@H](CC(C)(C)C)CO2] | 1.67 | 639.263 | 640.2 | A |
| 1854 | 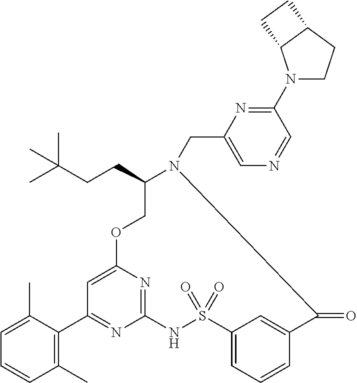 [SMILES: Cc1cccc(C)c1-c1cc2nc(n1)NS(=O)(=O)c1cccc(c1)C(=O)N(Cc1cncc(N3CC[C@H]4CC[C@H]43)n1)[C@H](CCC(C)(C)C)CO2] | 1.94 | 695.325 | 696.3 | A |
| 1855 | 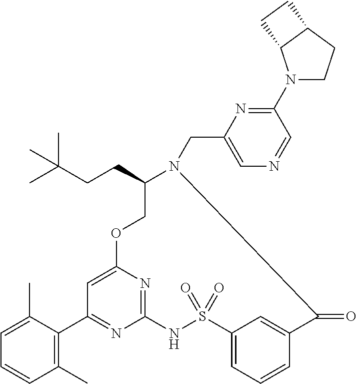 [SMILES: Cc1cccc(C)c1-c1cc2nc(n1)NS(=O)(=O)c1cccc(c1)C(=O)N(Cc1cncc(N3CC[C@H]4CC[C@H]43)n1)[C@H](CCC(C)(C)C)CO2] | 1.93 | 695.325 | 696.3 | A |
| 1856 | 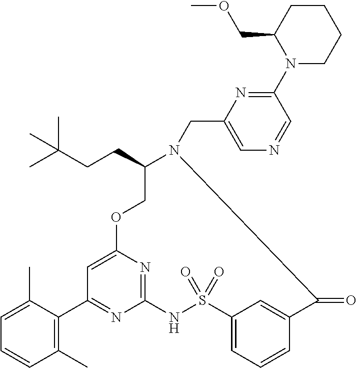 [SMILES: COC[C@H]1CCCCN1c1cncc(CN2C(=O)c3cccc(c3)S(=O)(=O)Nc3nc(cc(-c4c(C)cccc4C)n3)OC[C@H]2CCC(C)(C)C)n1] | 1.9 | 727.352 | 728.3 | A |
| 1857 | 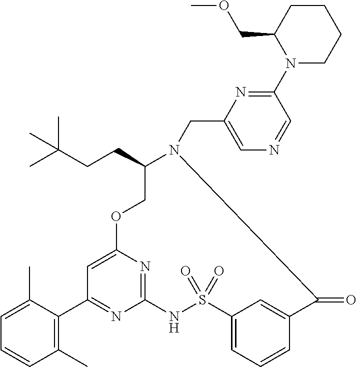 [SMILES: COC[C@H]1CCCCN1c1cncc(CN2C(=O)c3cccc(c3)S(=O)(=O)Nc3nc(cc(-c4c(C)cccc4C)n3)OC[C@H]2CCC(C)(C)C)n1] | 1.91 | 727.352 | 728.3 | A |
| 1858 | 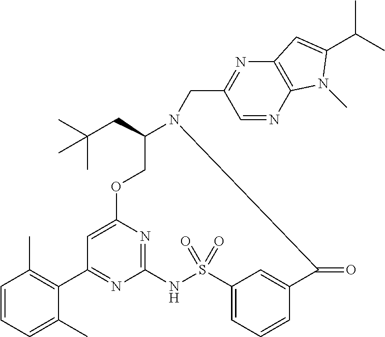 [SMILES: Cc1cccc(C)c1-c1cc2nc(n1)NS(=O)(=O)c1cccc(c1)C(=O)N(Cc1cnc3c(cc(C(C)C)n3C)n1)[C@H](CC(C)(C)C)CO2] | 2 | 681.31 | 682.2 | A |
| 1859 | 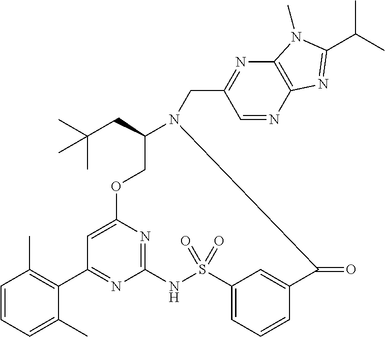 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC(C)(C)C)CO2] | 1.45 | 681.31 | 682.3 | A |
| 1329 | 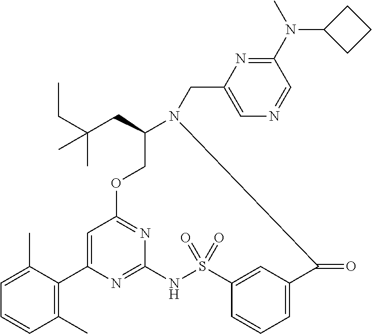 [SMILES: CCC(C)(C)C[C@@H]1COc2cc(-c3c(C)cccc3C)nc(n2)NS(=O)(=O)c2cccc(c2)C(=O)N1Cc1cncc(N(C)C2CCC2)n1] | 1.92 | 683.325 | 684.8 | A |
| 1330 | 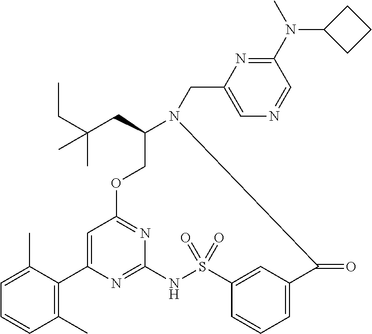 [SMILES: CCC(C)(C)C[C@@H]1COc2cc(-c3c(C)cccc3C)nc(n2)NS(=O)(=O)c2cccc(c2)C(=O)N1Cc1cncc(N(C)C2CCC2)n1] | 1.92 | 683.325 | 684.8 | A |
| 1860 | 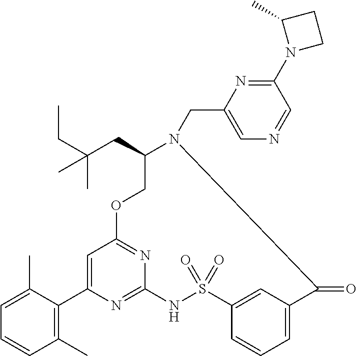 [SMILES: CCC(C)(C)C[C@@H]1COc2cc(-c3c(C)cccc3C)nc(n2)NS(=O)(=O)c2cccc(c2)C(=O)N1Cc1cncc(N2CC[C@H]2C)n1] | 1.87 | 669.31 | 670.8 | A |
| 1861 | 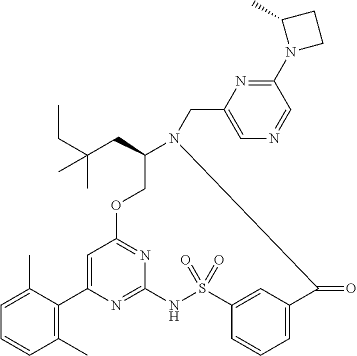 [SMILES: CCC(C)(C)C[C@@H]1COc2cc(-c3c(C)cccc3C)nc(n2)NS(=O)(=O)c2cccc(c2)C(=O)N1Cc1cncc(N2CC[C@H]2C)n1] | 1.87 | 669.31 | 670.7 | A |
| 1862 | 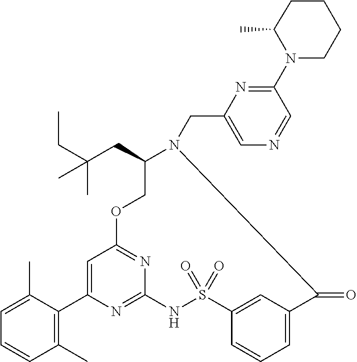 [SMILES: CCC(C)(C)C[C@@H]1COc2cc(-c3c(C)cccc3C)nc(n2)NS(=O)(=O)c2cccc(c2)C(=O)N1Cc1cncc(N2CCCC[C@H]2C)n1] | 1.93 | 697.341 | 698.8 | A |
| 1863 | 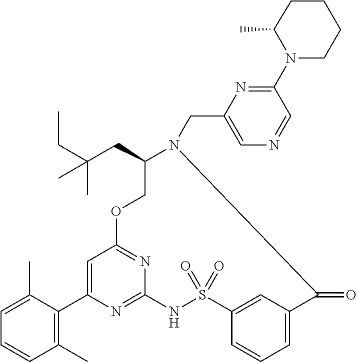 [SMILES: CCC(C)(C)C[C@@H]1COc2cc(-c3c(C)cccc3C)nc(n2)NS(=O)(=O)c2cccc(c2)C(=O)N1Cc1cncc(N2CCCC[C@H]2C)n1] | 1.93 | 697.341 | 698.8 | A |
| 1864 | 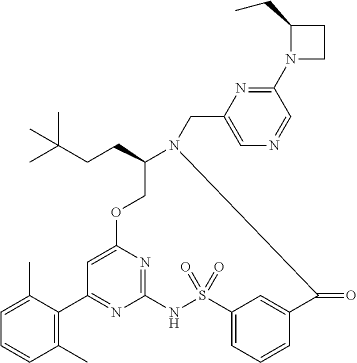 [SMILES: CC[C@H]1CCN1c1cncc(CN2C(=O)c3cccc(c3)S(=O)(=O)Nc3nc(cc(-c4c(C)cccc4C)n3)OC[C@H]2CCC(C)(C)C)n1] | 1.97 | 683.325 | 684.2 | A |
| 1865 | 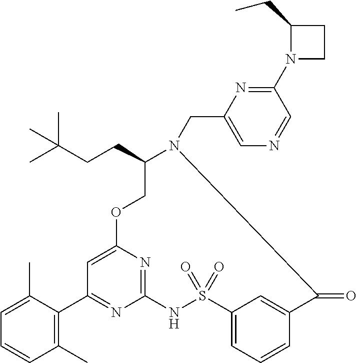 [SMILES: CC[C@H]1CCN1c1cncc(CN2C(=O)c3cccc(c3)S(=O)(=O)Nc3nc(cc(-c4c(C)cccc4C)n3)OC[C@H]2CCC(C)(C)C)n1] | 1.99 | 683.325 | 684.3 | A |
| 1331 | 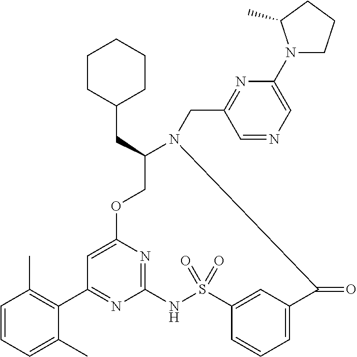 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CCCCC1)CO2] | 1.92 | 695.325 | 696.8 | A |
| 1866 | 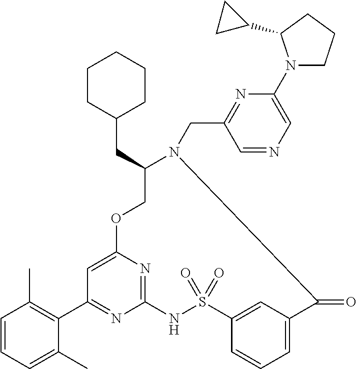 [SMILES: Cc1cccc(C)c1-c1cc2nc(n1)NS(=O)(=O)c1cccc(c1)C(=O)N(Cc1cncc(N3CCC[C@H]3C3CC3)n1)[C@H](CC1CCCCC1)CO2] | 2.02 | 721.341 | 722.8 | A |
| 1867 | 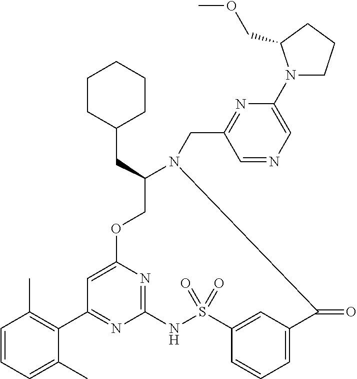 [SMILES: COC[C@@H]1CCCN1c1cncc(CN2C(=O)c3cccc(c3)S(=O)(=O)Nc3nc(cc(-c4c(C)cccc4C)n3)OC[C@H]2CC2CCCCC2)n1] | 1.9 | 725.336 | 726.8 | A |
| 1868 | 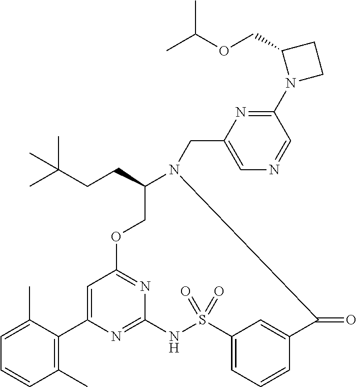 [SMILES: Cc1cccc(C)c1-c1cc2nc(n1)NS(=O)(=O)c1cccc(c1)C(=O)N(Cc1cncc(N3CC[C@H]3COC(C)C)n1)[C@H](CCC(C)(C)C)CO2] | 2 | 727.352 | 728.8 | A |
| 1869 | 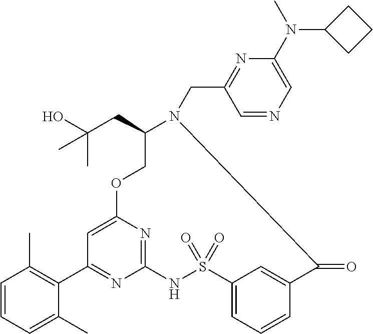 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)O)CO2] | 1.53 | 671.289 | 672.2 | A |
| 1332 | 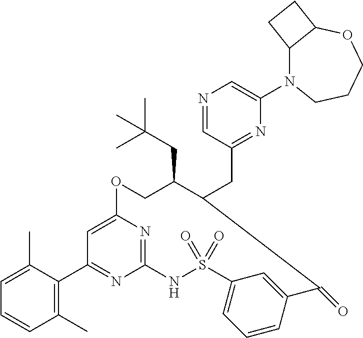 [SMILES: Cc1cccc(C)c1-c1cc2nc(n1)NS(=O)(=O)c1cccc(c1)C(=O)C(Cc1cncc(N3CCCOC4CCC43)n1)[C@H](CC(C)(C)C)CO2] | 4.36 | 711.32 | 712.2 | Y |
| 1870 | 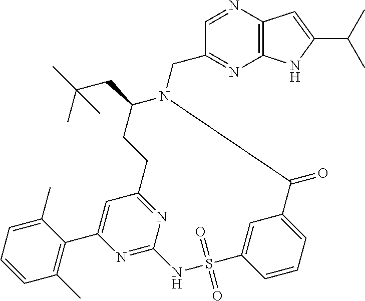 [SMILES: Cc1cccc(C)c1-c1cc2nc(n1)NS(=O)(=O)c1cccc(c1)C(=O)N(Cc1cnc3cc(C(C)C)[nH]c3n1)[C@H](CC(C)(C)C)CC2] | 4.31 | 667.294 | 668.2 | Y |
| 1871 | 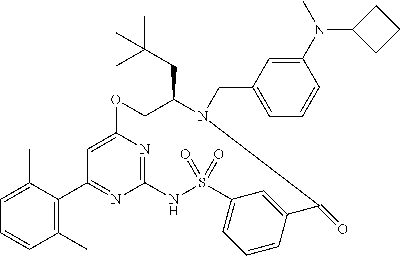 [SMILES: Cc1cccc(C)c1-c1cc2nc(n1)NS(=O)(=O)c1cccc(c1)C(=O)N(Cc1cccc(N(C)C3CCC3)c1)[C@H](CC(C)(C)C)CO2] | 3.6 | 667.319 | 668.2 | 1D |
| 1333 | 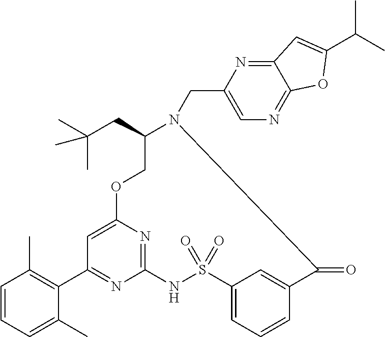 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)C)CO2] | 2.01 | 668.278 | 669.2 | A |
| 1334 | 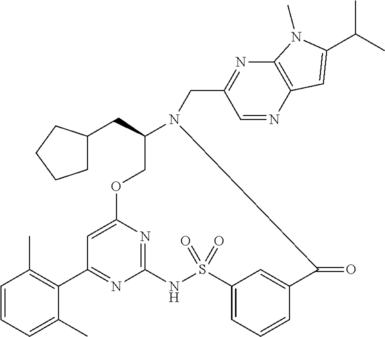 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCC1)CO2] | 1.8 | 693.31 | 694.3 | A |
| 1872 | 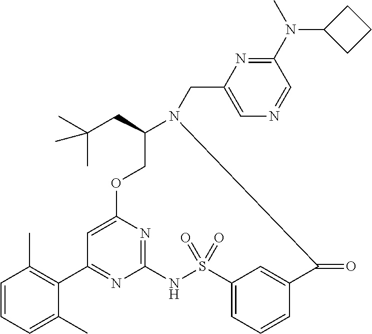 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)C)CO2] | 1.94 | 683.325 | 684.3 | A |
| 1873 | 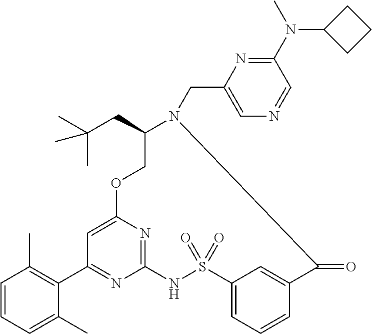 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)C)CO2] | 1.83 | 683.325 | 684.3 | A |
| 1874 | 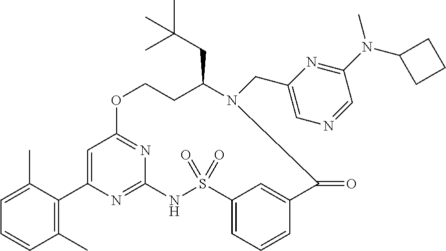 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)C)CCO2] | 1.86 | 683.325 | 684.3 | A |
| 1875 | 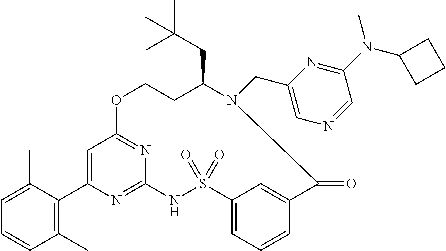 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)C)CCO2] | 1.84 | 683.325 | 684.2 | A |
| 1876 | 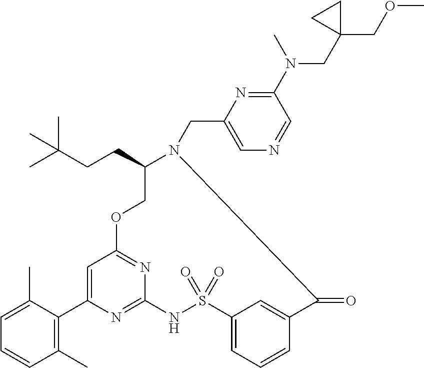 [SMILES: COCC1(CN(C)c2cncc(CN3C(=O)c4cccc(c4)S(=O)(=O)Nc4nc(cc(-c5c(C)cccc5C)n4)OC[C@H]3CCC(C)(C)C)n2)CC1] | 1.84 | 727.352 | 728.2 | A |
| 1877 | 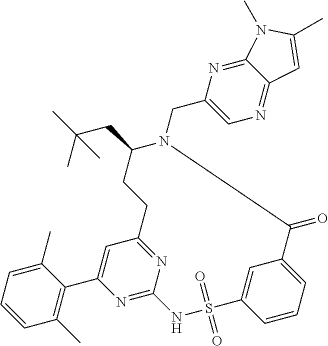 [SMILES: Cc1cccc(C)c1-c1cc2nc(n1)NS(=O)(=O)c1cccc(c1)C(=O)N(Cc1cnc3cc(C)n(C)c3n1)[C@H](CC(C)(C)C)CC2] | 4.27 | 653.278 | 654.2 | Y |
| 1878 | 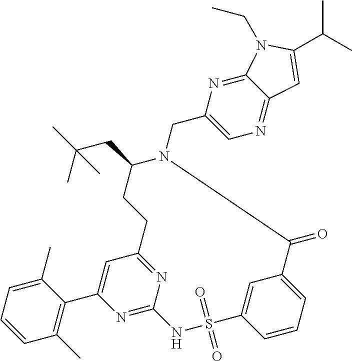 [SMILES: CCn1c(C(C)C)cc2ncc(CN3C(=O)c4cccc(c4)S(=O)(=O)Nc4nc(cc(-c5c(C)cccc5C)n4)CC[C@H]3CC(C)(C)C)nc21] | 3.37 | 695.325 | 696.2 | 1D |
| 1879 | 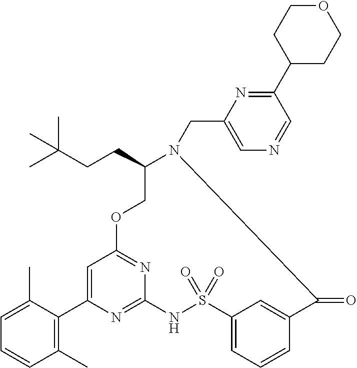 [SMILES: Cc1cccc(C)c1-c1cc2nc(n1)NS(=O)(=O)c1cccc(c1)C(=O)N(Cc1cncc(C3CCOCC3)n1)[C@H](CCC(C)(C)C)CO2] | 1.803 | 684.309 | 685.2 | A |
| 1880 | 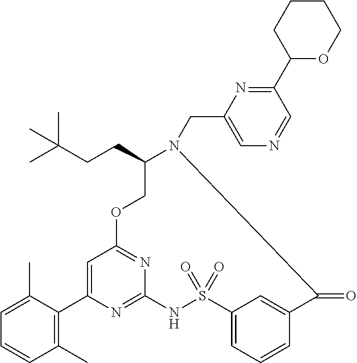 [SMILES: Cc1cccc(C)c1-c1cc2nc(n1)NS(=O)(=O)c1cccc(c1)C(=O)N(Cc1cncc(C3CCCCO3)n1)[C@H](CCC(C)(C)C)CO2] | 2.005 | 684.309 | 685.7 | A |
| 1881 | 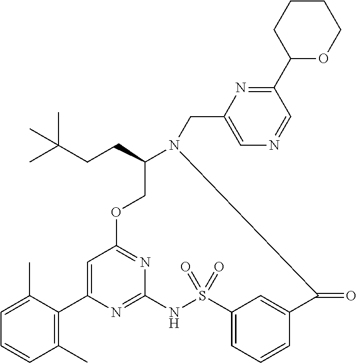 [SMILES: Cc1cccc(C)c1-c1cc2nc(n1)NS(=O)(=O)c1cccc(c1)C(=O)N(Cc1cncc(C3CCCCO3)n1)[C@H](CCC(C)(C)C)CO2] | 1.925 | 684.309 | 685.1 | A |
| 1335 | 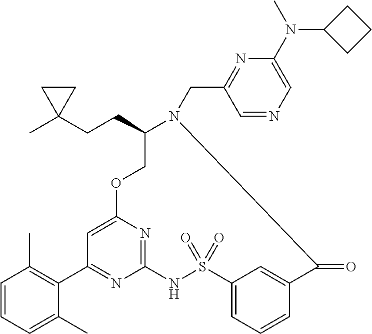 [SMILES: Cc1cccc(C)c1-c1cc2nc(n1)NS(=O)(=O)c1cccc(c1)C(=O)N(Cc1cncc(N(C)C3CCC3)n1)[C@H](CCC1(C)CC1)CO2] | 1.87 | 681.31 | 682.2 | A |
| 1336 | 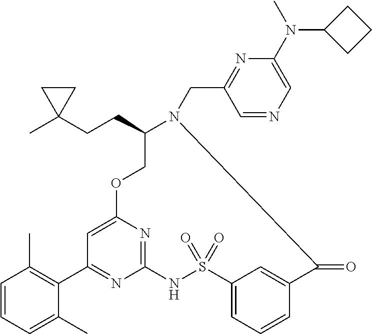 [SMILES: Cc1cccc(C)c1-c1cc2nc(n1)NS(=O)(=O)c1cccc(c1)C(=O)N(Cc1cncc(N(C)C3CCC3)n1)[C@H](CCC1(C)CC1)CO2] | 1.87 | 681.31 | 682.3 | A |
| 1882 | 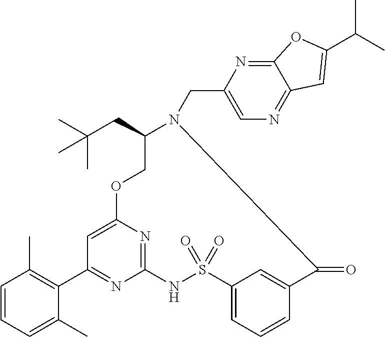 [SMILES: Cc1cccc(C)c1-c1cc2nc(n1)NS(=O)(=O)c1cccc(c1)C(=O)N(Cc1cnc3cc(C(C)C)oc3n1)[C@H](CC(C)(C)C)CO2] | 1.96 | 668.278 | 669.2 | A |
| 1883 | 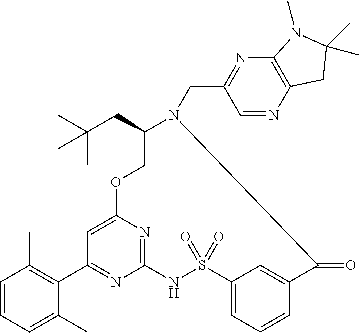 [SMILES: Cc1cccc(C)c1-c1cc2nc(n1)NS(=O)(=O)c1cccc(c1)C(=O)N(Cc1cnc3c(n1)N(C)C(C)(C)C3)[C@H](CC(C)(C)C)CO2] | 1.74 | 669.31 | 670.3 | A |
| 1884 | 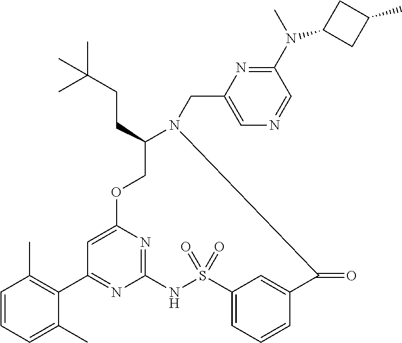 [SMILES: Cc1cccc(C)c1-c1cc2nc(n1)NS(=O)(=O)c1cccc(c1)C(=O)N(Cc1cncc(N(C)[C@H]3C[C@@H](C)C3)n1)[C@H](CCC(C)(C)C)CO2] | 2.05 | 697.341 | 698.8 | A |
| 1885 | 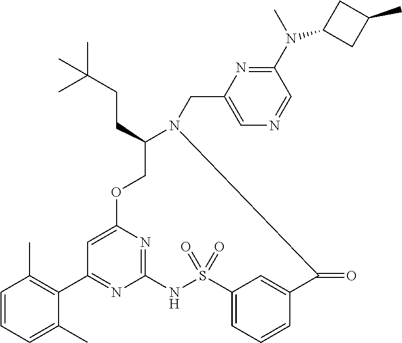 [SMILES: Cc1cccc(C)c1-c1cc2nc(n1)NS(=O)(=O)c1cccc(c1)C(=O)N(Cc1cncc(N(C)[C@H]3C[C@H](C)C3)n1)[C@H](CCC(C)(C)C)CO2] | 2.06 | 697.341 | 698.8 | A |
| 1886 | 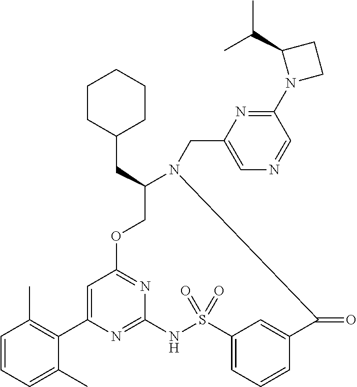 [SMILES: Cc1cccc(C)c1-c1cc2nc(n1)NS(=O)(=O)c1cccc(c1)C(=O)N(Cc1cncc(N3CC[C@@H]3C(C)C)n1)[C@H](CC1CCCCC1)CO2] | 2.09 | 709.341 | 710.8 | A |
| 1887 | 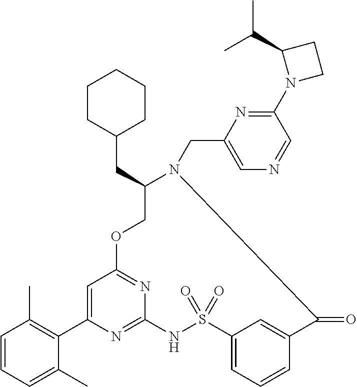 [SMILES: Cc1cccc(C)c1-c1cc2nc(n1)NS(=O)(=O)c1cccc(c1)C(=O)N(Cc1cncc(N3CC[C@@H]3C(C)C)n1)[C@H](CC1CCCCC1)CO2] | 2.11 | 709.341 | 710.8 | A |
| 1888 | 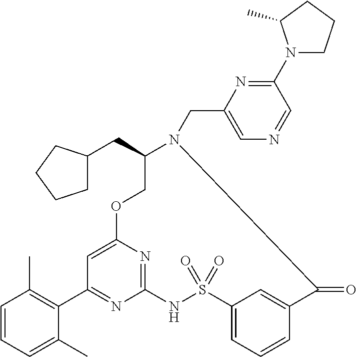 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CCCC1)CO2] | 1.81 | 681.31 | 682.2 | A |
| 1889 | 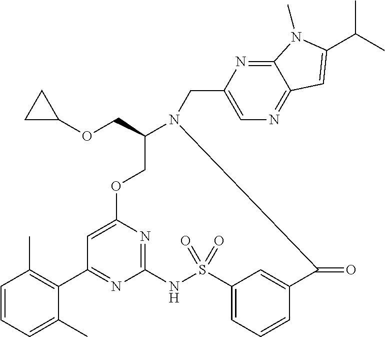 [SMILES: Cc1cccc(C)c1-c1cc2nc(n1)NS(=O)(=O)c1cccc(c1)C(=O)N(Cc1cnc3cc(C(C)C)n(C)c3n1)[C@H](COC1CC1)CO2] | 1.29 | 681.273 | 682.2 | A |
| 1890 | 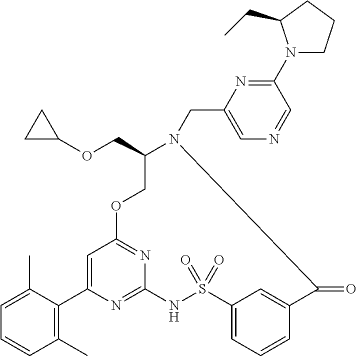 [SMILES: CC[C@H]1CCCN1c1cncc(CN2C(=O)c3cccc(c3)S(=O)(=O)Nc3nc(cc(-c4c(C)cccc4C)n3)OC[C@H]2COC2CC2)n1] | 1.67 | 683.289 | 684.2 | A |
| 1891 | 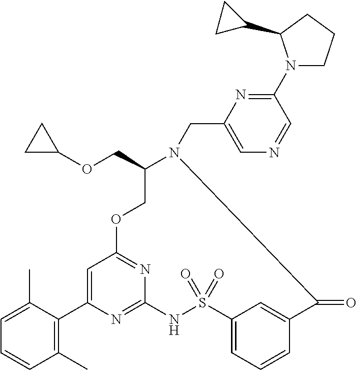 [SMILES: Cc1cccc(C)c1-c1cc2nc(n1)NS(=O)(=O)c1cccc(c1)C(=O)N(Cc1cncc(N3CCC[C@@H]3C3CC3)n1)[C@H](COC1CC1)CO2] | 1.69 | 695.289 | 696.5 | A |
| 1892 | 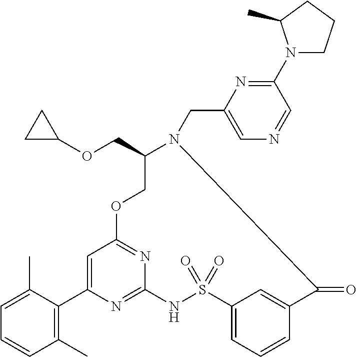 [SMILES: Cc1cccc(C)c1-c1cc2nc(n1)NS(=O)(=O)c1cccc(c1)C(=O)N(Cc1cncc(N3CCC[C@@H]3C)n1)[C@H](COC1CC1)CO2] | 1.565 | 669.273 | 670.2 | A |
| 1893 | 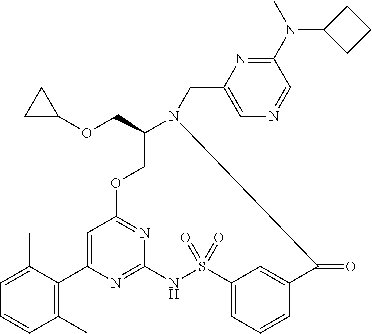 [SMILES: Cc1cccc(C)c1-c1cc2nc(n1)NS(=O)(=O)c1cccc(c1)C(=O)N(Cc1cncc(N(C)C3CCC3)n1)[C@H](COC1CC1)CO2] | 1.628 | 669.273 | 670.3 | A |
| 1894 | 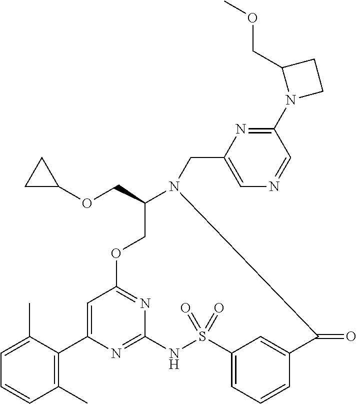 [SMILES: COCC1CCN1c1cncc(CN2C(=O)c3cccc(c3)S(=O)(=O)Nc3nc(cc(-c4c(C)cccc4C)n3)OC[C@H]2COC2CC2)n1] | 1.532 | 685.268 | 686.4 | A |
| 1895 | 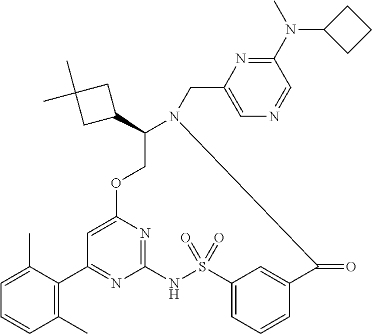 [SMILES: Cc1cccc(C)c1-c1cc2nc(n1)NS(=O)(=O)c1cccc(c1)C(=O)N(Cc1cncc(N(C)C3CCC3)n1)[C@H](C1CC(C)(C)C1)CO2] | 1.863 | 681.31 | 682.2 | A |
| 1896 | 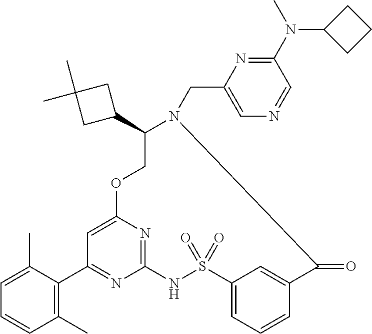 [SMILES: Cc1cccc(C)c1-c1cc2nc(n1)NS(=O)(=O)c1cccc(c1)C(=O)N(Cc1cncc(N(C)C3CCC3)n1)[C@H](C1CC(C)(C)C1)CO2] | 1.867 | 681.31 | 682.2 | A |
| 1337 | 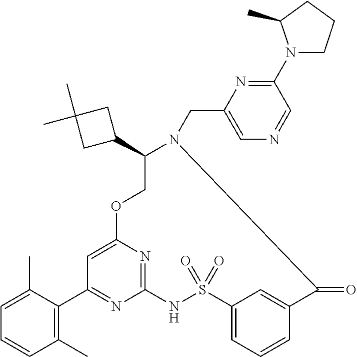 [SMILES: Cc1cccc(C)c1-c1cc2nc(n1)NS(=O)(=O)c1cccc(c1)C(=O)N(Cc1cncc(N3CCC[C@@H]3C)n1)[C@H](C1CC(C)(C)C1)CO2] | 1.795 | 681.31 | 682.2 | A |
| 1338 | 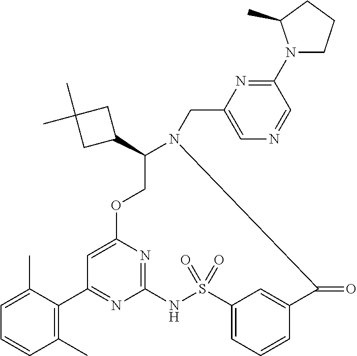 [SMILES: Cc1cccc(C)c1-c1cc2nc(n1)NS(=O)(=O)c1cccc(c1)C(=O)N(Cc1cncc(N3CCC[C@@H]3C)n1)[C@H](C1CC(C)(C)C1)CO2] | 1.805 | 681.31 | 682.3 | A |
| 1897 | 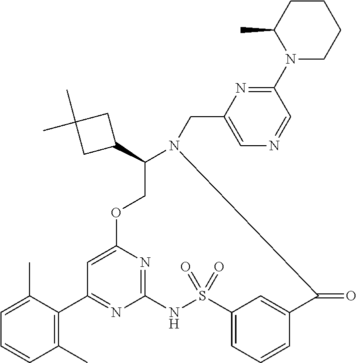 [SMILES: Cc1cccc(C)c1-c1cc2nc(n1)NS(=O)(=O)c1cccc(c1)C(=O)N(Cc1cncc(N3CCCC[C@@H]3C)n1)[C@H](C1CC(C)(C)C1)CO2] | 1.873 | 695.325 | 696.3 | A |
| 1898 | 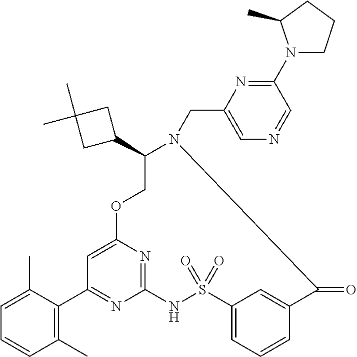 [SMILES: Cc1cccc(C)c1-c1cc2nc(n1)NS(=O)(=O)c1cccc(c1)C(=O)N(Cc1cncc(N3CCC[C@@H]3C)n1)[C@H](C1CC(C)(C)C1)CO2] | 1.877 | 695.325 | 696.3 | A |
| 1339 | 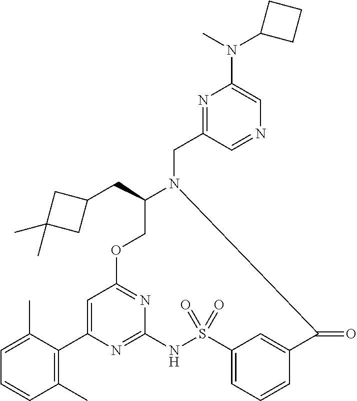 [SMILES: Cc1cccc(C)c1-c1cc2nc(n1)NS(=O)(=O)c1cccc(c1)C(=O)N(Cc1cncc(N(C)C3CCC3)n1)[C@H](CC1CC(C)(C)C1)CO2] | 1.73 | 695.325 | 696.6 | A |
| 1340 | 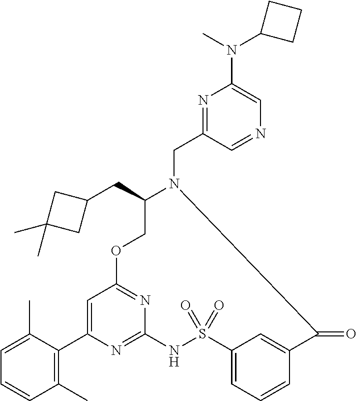 [SMILES: Cc1cccc(C)c1-c1cc2nc(n1)NS(=O)(=O)c1cccc(c1)C(=O)N(Cc1cncc(N(C)C3CCC3)n1)[C@H](CC1CC(C)(C)C1)CO2] | 1.73 | 695.325 | 696.6 | A |
| 1899 | 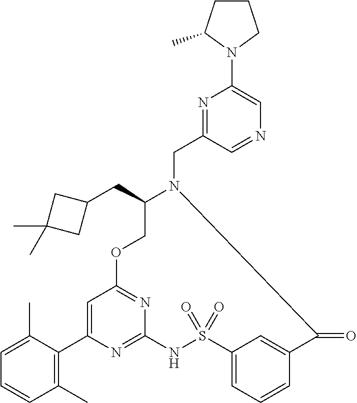 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CC(C)(C)C1)CO2] | 1.67 | 695.325 | 696.5 | A |
| 1900 | 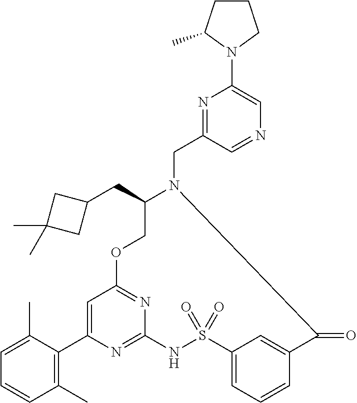 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CC(C)(C)C1)CO2] | 1.67 | 695.325 | 696.5 | A |
| 1901 | 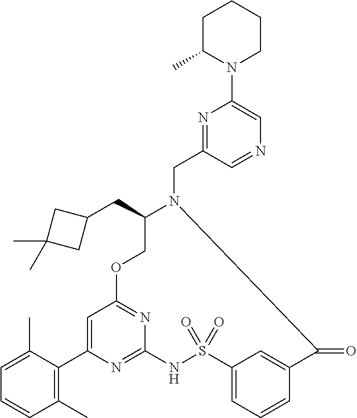 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CC(C)(C)C1)CO2] | 1.75 | 709.341 | 710.5 | A |
| 1902 | 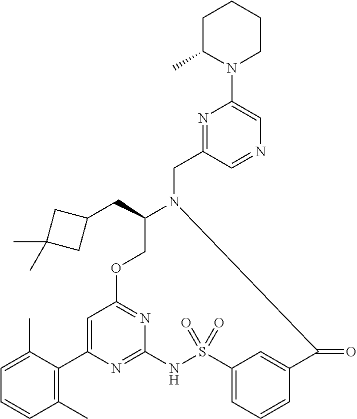 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CC(C)(C)C1)CO2] | 1.74 | 709.341 | 710.5 | A |
| 1903 | 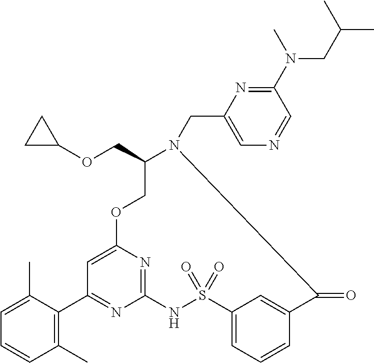 [SMILES: Cc1cccc(C)c1-c1cc2nc(n1)NS(=O)(=O)c1cccc(c1)C(=O)N(Cc1cncc(N(C)CC(C)C)n1)[C@H](COC1CC1)CO2] | 1.72 | 671.289 | 672.7 | A |
| 1904 | 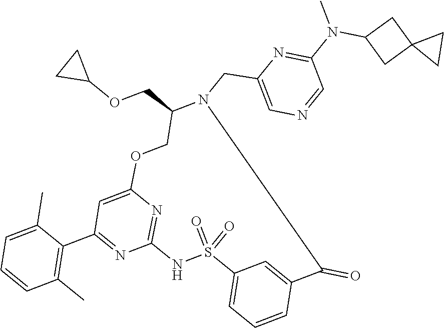 [SMILES: Cc1cccc(C)c1-c1cc2nc(n1)NS(=O)(=O)c1cccc(c1)C(=O)N(Cc1cncc(N(C)C3CC4(CC4)C3)n1)[C@H](COC1CC1)CO2] | 1.82 | 695.289 | 696.8 | A |
| 1905 | 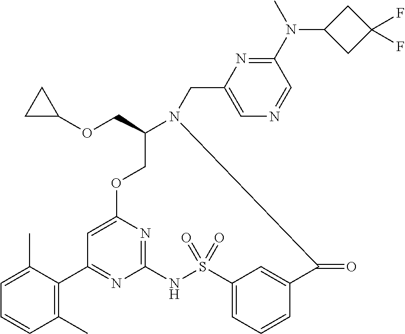 [SMILES: Cc1cccc(C)c1-c1cc2nc(n1)NS(=O)(=O)c1cccc(c1)C(=O)N(Cc1cncc(N(C)C3CC(F)(F)C3)n1)[C@H](COC1CC1)CO2] | 1.72 | 705.254 | 706.7 | A |
| 1341 | 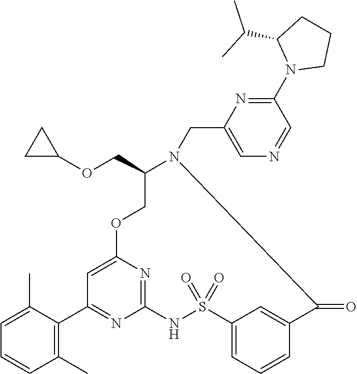 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](COC1CC1)CO2] | 1.81 | 697.305 | 698.7 | A |
| 1906 | 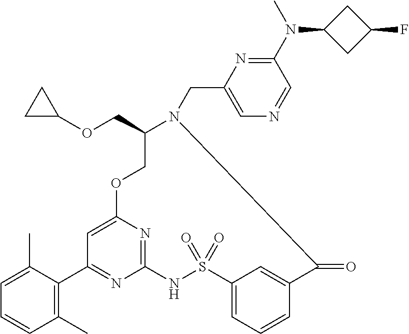 [SMILES: Cc1cccc(C)c1-c1cc2nc(n1)NS(=O)(=O)c1cccc(c1)C(=O)N(Cc1cncc(N(C)[C@H]3C[C@@H](F)C3)n1)[C@H](COC1CC1)CO2] | 1.65 | 687.264 | 688.7 | A |
| 1907 | 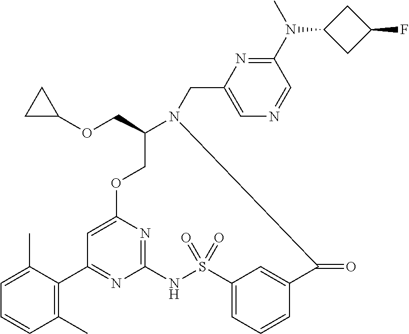 [SMILES: Cc1cccc(C)c1-c1cc2nc(n1)NS(=O)(=O)c1cccc(c1)C(=O)N(Cc1cncc(N(C)[C@H]3C[C@H](F)C3)n1)[C@H](COC1CC1)CO2] | 1.64 | 687.264 | 688.7 | A |
| 1908 | 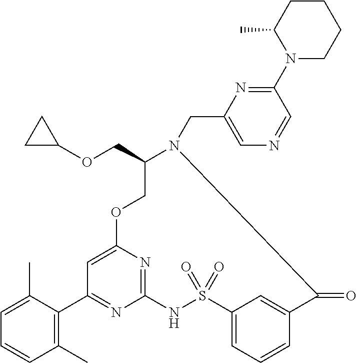 [SMILES: Cc1cccc(C)c1-c1cc2nc(n1)NS(=O)(=O)c1cccc(c1)C(=O)N(Cc1cncc(N3CCCC[C@H]3C)n1)[C@H](COC1CC1)CO2] | 1.665 | 683.289 | 684.3 | A |
| 1909 | 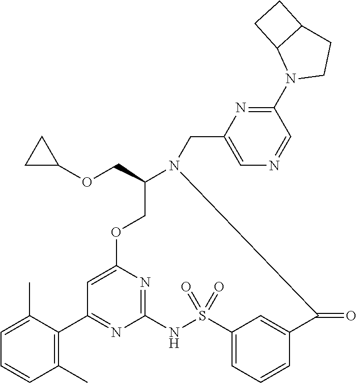 [SMILES: Cc1cccc(C)c1-c1cc2nc(n1)NS(=O)(=O)c1cccc(c1)C(=O)N(Cc1cncc(N3CCC4CCC43)n1)[C@H](COC1CC1)CO2] | 1.71 | 681.273 | 682.7 | A |
| 1910 | 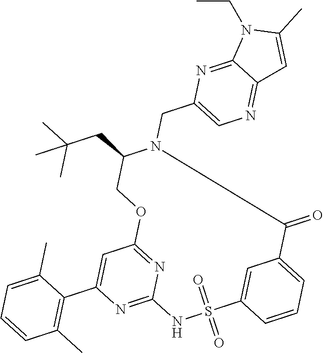 [SMILES: CCn1c(C)cc2ncc(CN3C(=O)c4cccc(c4)S(=O)(=O)Nc4nc(cc(-c5c(C)cccc5C)n4)OC[C@H]3CC(C)(C)C)nc21] | 4.39 | 667.294 | 668.2 | Y |
| 1911 | 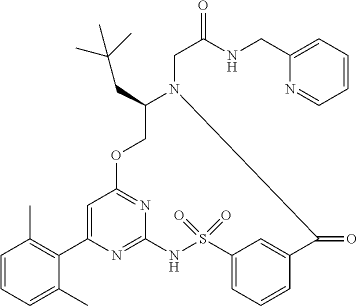 [SMILES: Cc1cccc(C)c1-c1cc2nc(n1)NS(=O)(=O)c1cccc(c1)C(=O)N(CC(=O)NCc1ccccn1)[C@H](CC(C)(C)C)CO2] | 4.46 | 667.294 | 668.3 | Y |
| 1912 | 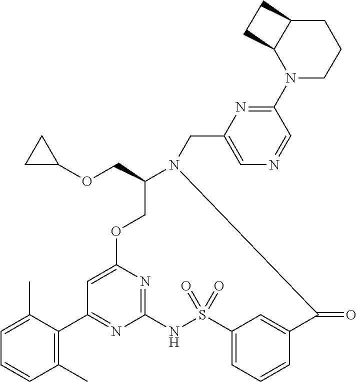 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](COC1CC1)CO2] | 1.81 | 695.289 | 696.7 | A |
| 1913 | 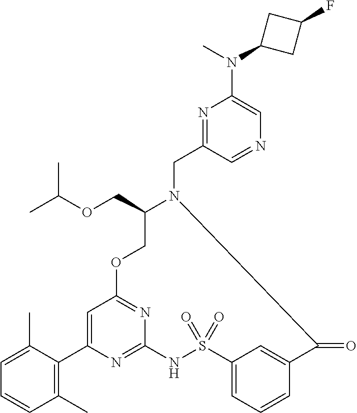 [SMILES: Cc1cccc(C)c1-c1cc2nc(n1)NS(=O)(=O)c1cccc(c1)C(=O)N(Cc1cncc(N(C)[C@H]3C[C@@H](F)C3)n1)[C@H](COC(C)C)CO2] | 1.6 | 687.3 | 688.5 | A |
| 1914 | 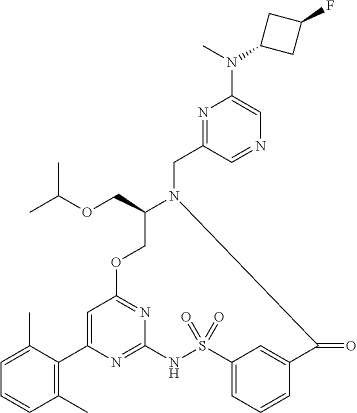 [SMILES: Cc1cccc(C)c1-c1cc2nc(n1)NS(=O)(=O)c1cccc(c1)C(=O)N(Cc1cncc(N(C)[C@H]3C[C@H](F)C3)n1)[C@H](COC(C)C)CO2] | 1.6 | 687.3 | 688.5 | A |
| 1915 | 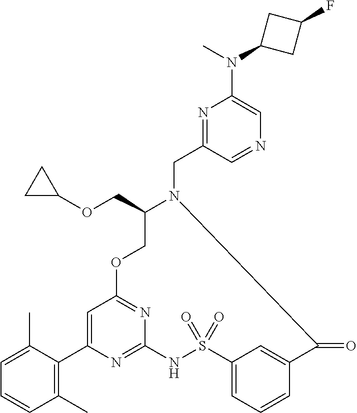 [SMILES: Cc1cccc(C)c1-c1cc2nc(n1)NS(=O)(=O)c1cccc(c1)C(=O)N(Cc1cncc(N(C)[C@H]3C[C@@H](F)C3)n1)[C@H](COC1CC1)CO2] | 1.54 | 685.285 | 686.5 | A |
| 1916 | 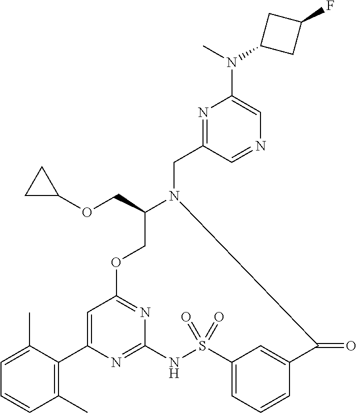 [SMILES: Cc1cccc(C)c1-c1cc2nc(n1)NS(=O)(=O)c1cccc(c1)C(=O)N(Cc1cncc(N(C)[C@H]3C[C@H](F)C3)n1)[C@H](COC1CC1)CO2] | 1.53 | 685.285 | 686.6 | A |
| 1917 | 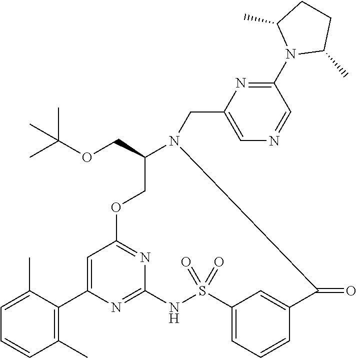 [SMILES: Cc1cccc(C)c1-c1cc2nc(n1)NS(=O)(=O)c1cccc(c1)C(=O)N(Cc1cncc(N3[C@H](C)CC[C@@H]3C)n1)[C@H](COC(C)(C)C)CO2] | 1.9 | 697.341 | 698.3 | A |
| 1918 | 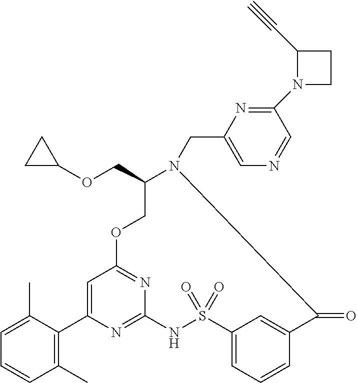 [SMILES: C#CC1CCN1c1cncc(CN2C(=O)c3cccc(c3)S(=O)(=O)Nc3nc(cc(-c4c(C)cccc4C)n3)OC[C@H]2COC2CC2)n1] | 1.59 | 655.258 | 656.2 | A |
| 1919 | 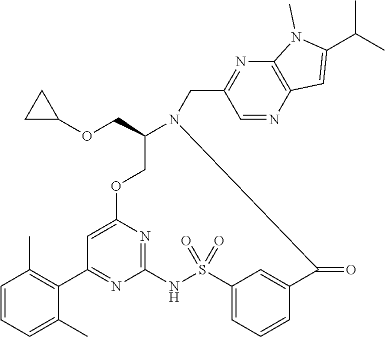 [SMILES: Cc1cccc(C)c1-c1cc2nc(n1)NS(=O)(=O)c1cccc(c1)C(=O)N(Cc1cnc3cc(C(C)C)n(C)c3n1)[C@H](COC1CC1)CO2] | 1.65 | 683.289 | 684.2 | A |
| 1920 | 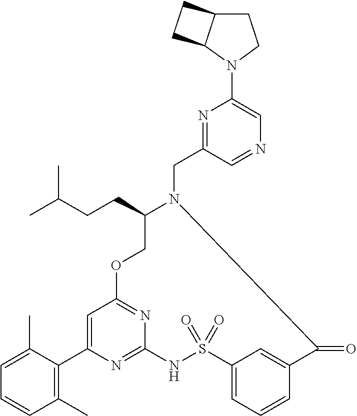 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](CCC(C)C)CO2] | 1.66 | 681.31 | 682.5 | A |
| 1921 | 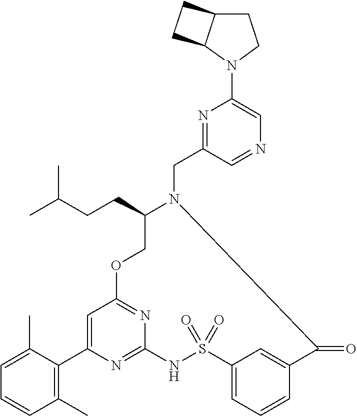 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](CCC(C)C)CO2] | 1.64 | 681.31 | 682.5 | A |
| 1922 | 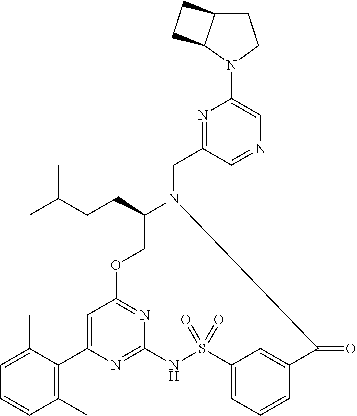 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](CCC(C)C)CO2] | 1.59 | 679.294 | 680.5 | A |
| 1923 | 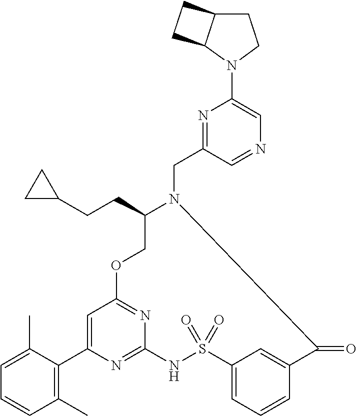 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](CCC1CC1)CO2] | 1.58 | 679.294 | 680.6 | A |
| 1924 | 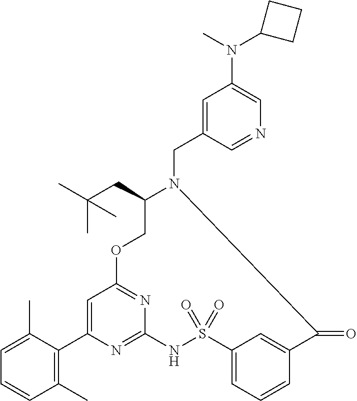 [SMILES: Cc1cccc(C)c1-c1cc2nc(n1)NS(=O)(=O)c1cccc(c1)C(=O)N(Cc1cncc(N(C)C3CCC3)c1)[C@H](CC(C)(C)C)CO2] | 2.22 | 669.31 | 670.6 | W |
| 1925 | 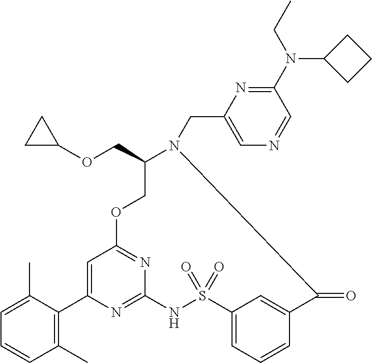 [SMILES: CCN(c1cncc(CN2C(=O)c3cccc(c3)S(=O)(=O)Nc3nc(cc(-c4c(C)cccc4C)n3)OC[C@H]2COC2CC2)n1)C1CCC1] | 1.7 | 683.289 | 684.2 | A |
| 1926 | 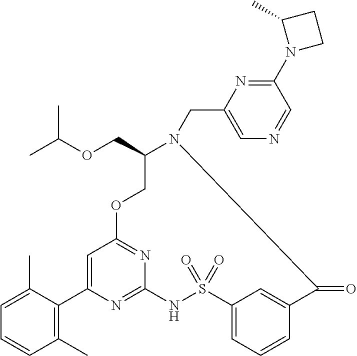 [SMILES: Cc1cccc(C)c1-c1cc2nc(n1)NS(=O)(=O)c1cccc(c1)C(=O)N(Cc1cncc(N3CC[C@H]3C)n1)[C@H](COC(C)C)CO2] | 1.63 | 657.273 | 658.2 | A |
| 1927 | 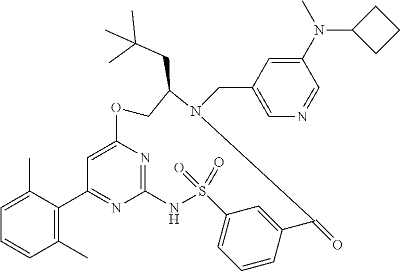 [SMILES: Cc1cccc(C)c1-c1cc2nc(n1)NS(=O)(=O)c1cccc(c1)C(=O)N(Cc1cncc(N(C)C3CCC3)c1)[C@H](CC(C)(C)C)CO2] | 3.07 | 668.314 | 669.2 | Y |
| 1928 | 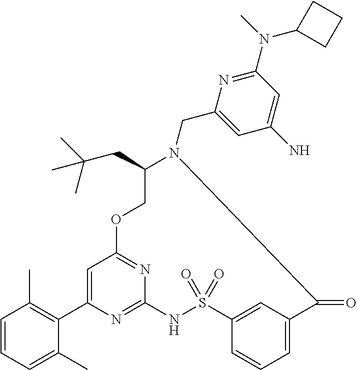 [SMILES: Cc1cccc(C)c1-c1cc2nc(n1)NS(=O)(=O)c1cccc(c1)C(=O)N(Cc1cc(N)cc(N(C)C3CCC3)n1)[C@H](CC(C)(C)C)CO2] | 2.84 | 683.325 | 684.3 | Y |
| 1929 | 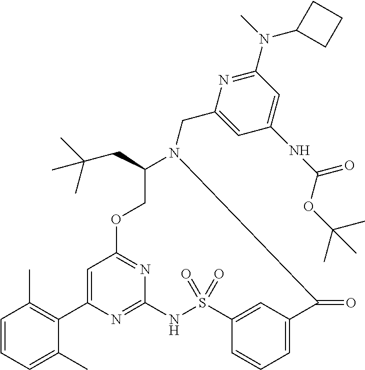 [SMILES: Cc1cccc(C)c1-c1cc2nc(n1)NS(=O)(=O)c1cccc(c1)C(=O)N(Cc1cc(NC(=O)OC(C)(C)C)cc(N(C)C3CCC3)n1)[C@H](CC(C)(C)C)CO2] | 3.66 | 783.378 | 784.3 | Y |
| 1930 | 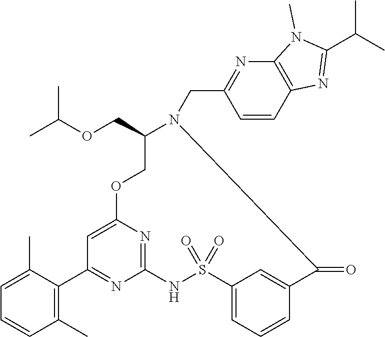 [SMILES: Cc1cccc(C)c1-c1cc2nc(n1)NS(=O)(=O)c1cccc(c1)C(=O)N(Cc1ccc3nc(C(C)C)n(C)c3n1)[C@H](COC(C)C)CO2] | 1.34 | 683.289 | 684.2 | A |
| 1931 | 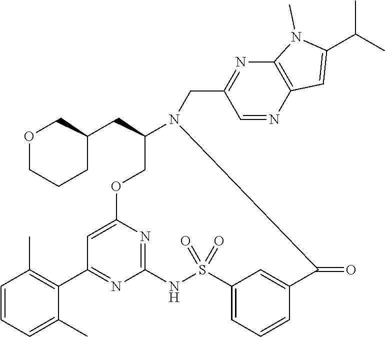 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@H]1CCCOC1)CO2] | 1.5 | 709.305 | 710.3 | A |
| 1932 | 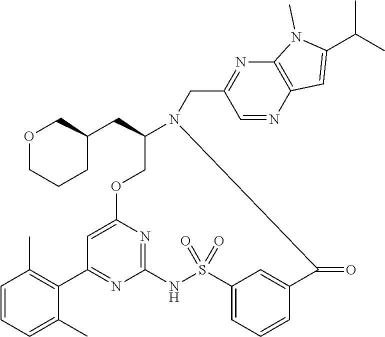 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@H]1CCCOC1)CO2] | 1.57 | 709.305 | 710.3 | A |
| 1933 | 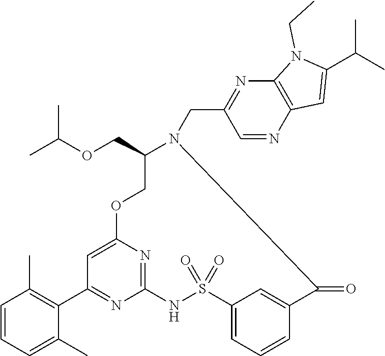 [SMILES: CCn1c(C(C)C)cc2ncc(CN3C(=O)c4cccc(c4)S(=O)(=O)Nc4nc(cc(-c5c(C)cccc5C)n4)OC[C@H]3COC(C)C)nc21] | 1.71 | 697.305 | 698.3 | A |
| 1934 | 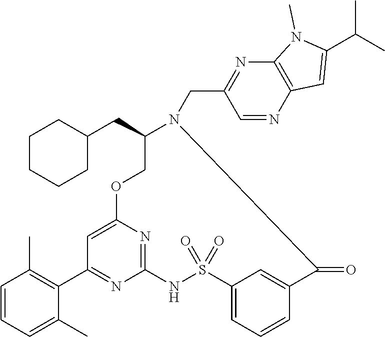 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCCC1)CO2] | 1.88 | 707.325 | 708.3 | A |
| 1935 | 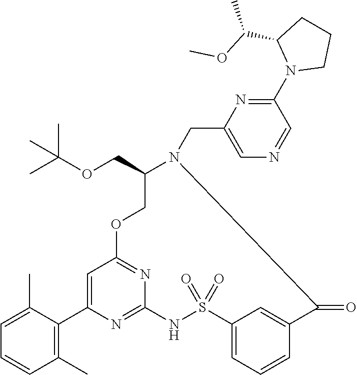 [SMILES: CO[C@H](C)[C@@H]1CCCN1c1cncc(CN2C(=O)c3cccc(c3)S(=O)(=O)Nc3nc(cc(-c4c(C)cccc4C)n3)OC[C@H]2COC(C)(C)C)n1] | 1.87 | 727.352 | 728.3 | A |
| 1936 | 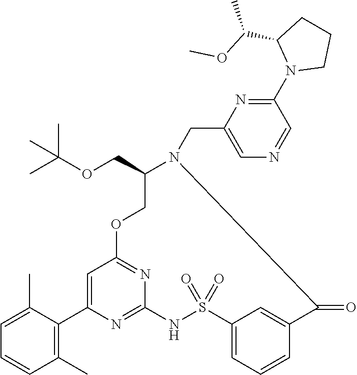 [SMILES: CO[C@H](C)[C@@H]1CCCN1c1cncc(CN2C(=O)c3cccc(c3)S(=O)(=O)Nc3nc(cc(-c4c(C)cccc4C)n3)OC[C@H]2COC(C)(C)C)n1] | 1.84 | 727.352 | 728.4 | A |
| 1937 | 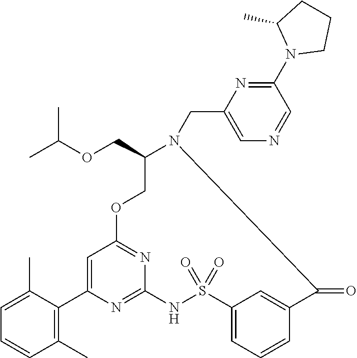 [SMILES: Cc1cccc(C)c1-c1cc2nc(n1)NS(=O)(=O)c1cccc(c1)C(=O)N(Cc1cncc(N3CCC[C@H]3C)n1)[C@H](COC(C)C)CO2] | 1.62 | 671.289 | 672.1 | A |
| 1938 | 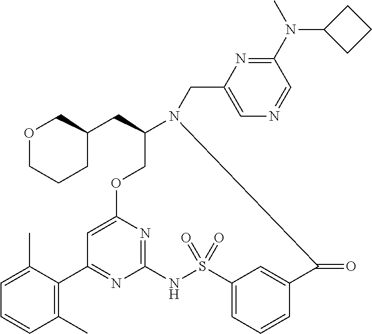 [SMILES: Cc1cccc(C)c1-c1cc2nc(n1)NS(=O)(=O)c1cccc(c1)C(=O)N(Cc1cncc(N(C)C3CCC3)n1)[C@H](C[C@H]1CCCOC1)CO2] | 1.61 | 697.305 | 698.8 | A |
| 1939 | 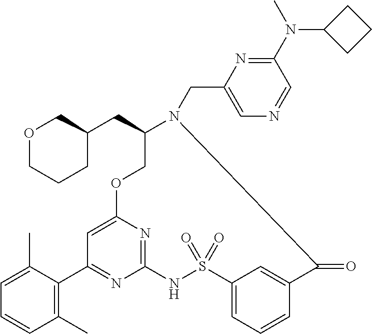 [SMILES: Cc1cccc(C)c1-c1cc2nc(n1)NS(=O)(=O)c1cccc(c1)C(=O)N(Cc1cncc(N(C)C3CCC3)n1)[C@H](C[C@H]1CCCOC1)CO2] | 1.67 | 697.305 | 698.8 | A |
| 1940 | 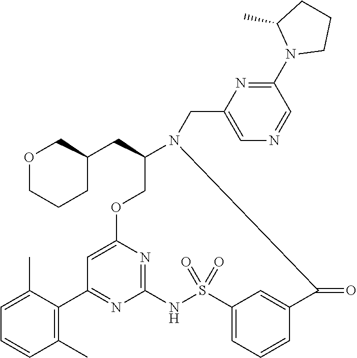 [SMILES: Cc1cccc(C)c1-c1cc2nc(n1)NS(=O)(=O)c1cccc(c1)C(=O)N(Cc1cncc(N3CCC[C@H]3C)n1)[C@H](C[C@H]1CCCOC1)CO2] | 1.55 | 697.305 | 698.7 | A |
| 1941 | 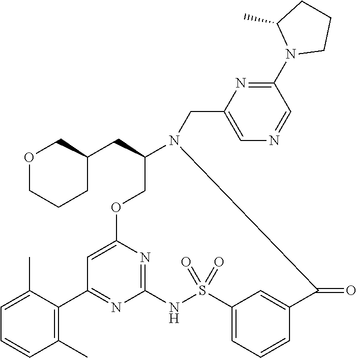 [SMILES: Cc1cccc(C)c1-c1cc2nc(n1)NS(=O)(=O)c1cccc(c1)C(=O)N(Cc1cncc(N3CCC[C@H]3C)n1)[C@H](C[C@H]1CCCOC1)CO2] | 1.6 | 697.305 | 698.8 | A |
| 1942 | 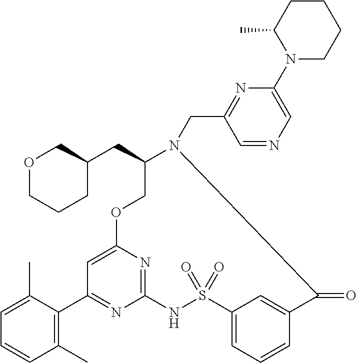 [SMILES: Cc1cccc(C)c1-c1cc2nc(n1)NS(=O)(=O)c1cccc(c1)C(=O)N(Cc1cncc(N3CCCC[C@H]3C)n1)[C@H](C[C@H]1CCCOC1)CO2] | 1.63 | 711.32 | 712.8 | A |
| 1943 | 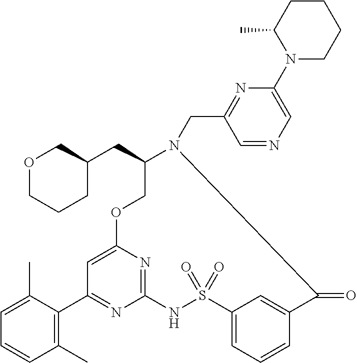 [SMILES: Cc1cccc(C)c1-c1cc2nc(n1)NS(=O)(=O)c1cccc(c1)C(=O)N(Cc1cncc(N3CCCC[C@H]3C)n1)[C@H](C[C@H]1CCCOC1)CO2] | 1.69 | 711.32 | 712.7 | A |
| 1944 | 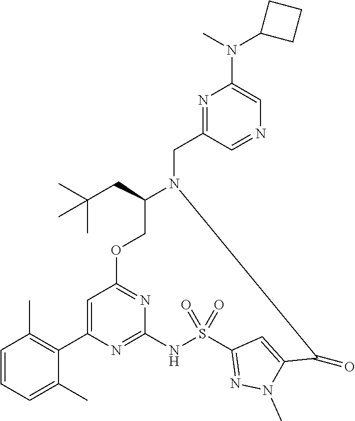 [SMILES: Cc1cccc(C)c1-c1cc2nc(n1)NS(=O)(=O)c1cc(n(C)n1)C(=O)N(Cc1cncc(N(C)C3CCC3)n1)[C@H](CC(C)(C)C)CO2] | 1.55 | 673.316 | 674.6 | A |
| 1945 | 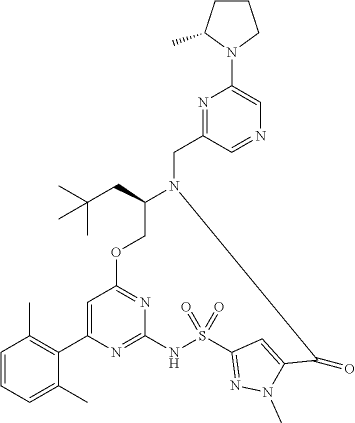 [SMILES: Cc1cccc(C)c1-c1cc2nc(n1)NS(=O)(=O)c1cc(n(C)n1)C(=O)N(Cc1cncc(N3CCC[C@H]3C)n1)[C@H](CC(C)(C)C)CO2] | 1.52 | 673.316 | 674.6 | A |
| 1946 | 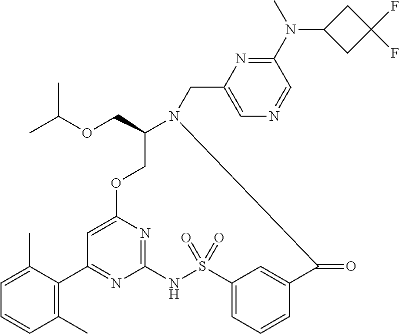 [SMILES: Cc1cccc(C)c1-c1cc2nc(n1)NS(=O)(=O)c1cccc(c1)C(=O)N(Cc1cncc(N(C)C3CC(F)(F)C3)n1)[C@H](COC(C)C)CO2] | 1.7 | 707.27 | 708.3 | A |
| 1947 | 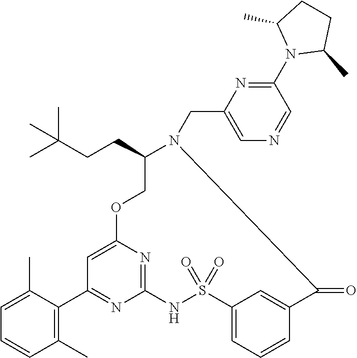 [SMILES: Cc1cccc(C)c1-c1cc2nc(n1)NS(=O)(=O)c1cccc(c1)C(=O)N(Cc1cncc(N3[C@H](C)CC[C@H]3C)n1)[C@H](CCC(C)(C)C)CO2] | 1.87 | 697.341 | 698.2 | A |
| 1948 | 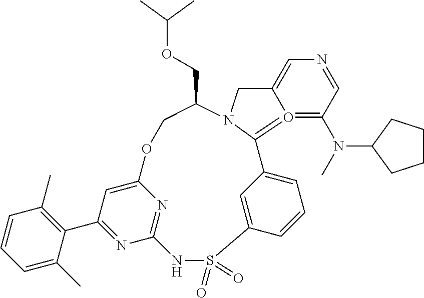 | 1.75 | 685.305 | 686.2 | A |
| 1949 | 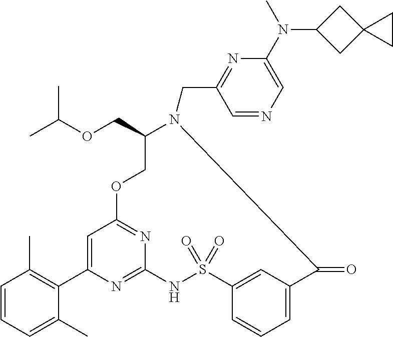 [SMILES: Cc1cccc(C)c1-c1cc2nc(n1)NS(=O)(=O)c1cccc(c1)C(=O)N(Cc1cncc(N(C)C3CC4(CC4)C3)n1)[C@H](COC(C)C)CO2] | 1.83 | 697.305 | 698.2 | A |
| 1950 | 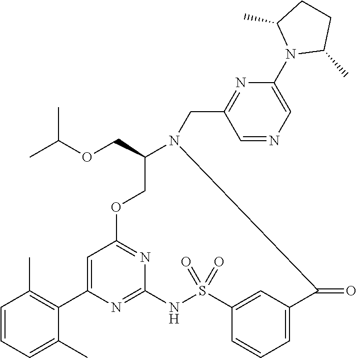 [SMILES: Cc1cccc(C)c1-c1cc2nc(n1)NS(=O)(=O)c1cccc(c1)C(=O)N(Cc1cncc(N3[C@H](C)CC[C@@H]3C)n1)[C@H](COC(C)C)CO2] | 1.72 | 685.305 | 686.2 | A |
| 1951 | 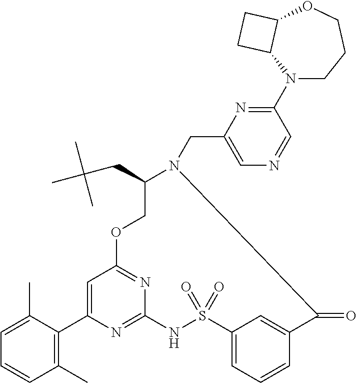 [SMILES: Cc1cccc(C)c1-c1cc2nc(n1)NS(=O)(=O)c1cccc(c1)C(=O)N(Cc1cncc(N3CCCO[C@H]4CC[C@H]43)n1)[C@H](CC(C)(C)C)CO2] | 1.76 | 711.32 | 712.79 | A |
| 1952 | 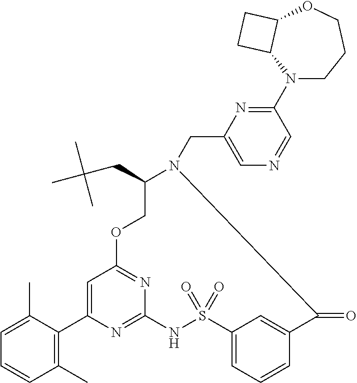 [SMILES: Cc1cccc(C)c1-c1cc2nc(n1)NS(=O)(=O)c1cccc(c1)C(=O)N(Cc1cncc(N3CCCO[C@H]4CC[C@H]43)n1)[C@H](CC(C)(C)C)CO2] | 1.74 | 711.32 | 712.73 | A |
| 1953 | 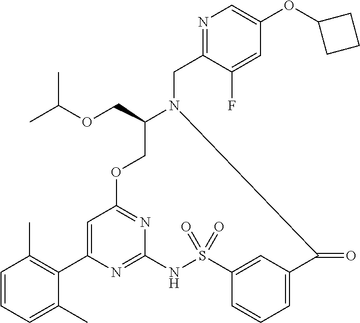 [SMILES: Cc1cccc(C)c1-c1cc2nc(n1)NS(=O)(=O)c1cccc(c1)C(=O)N(Cc1ncc(OC3CCC3)cc1F)[C@H](COC(C)C)CO2] | 1.74 | 675.253 | 676.4 | A |
| 1954 | 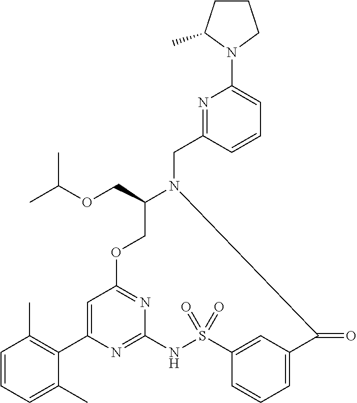 [SMILES: Cc1cccc(C)c1-c1cc2nc(n1)NS(=O)(=O)c1cccc(c1)C(=O)N(Cc1cccc(N3CCC[C@H]3C)n1)[C@H](COC(C)C)CO2] | 1.13 | 670.294 | 671.5 | A |
| 1955 | 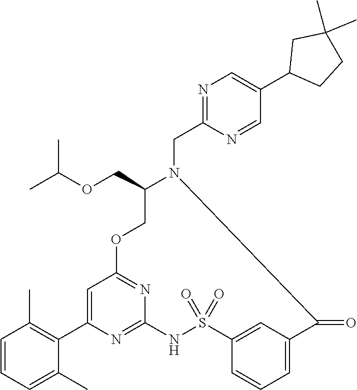 [SMILES: Cc1cccc(C)c1-c1cc2nc(n1)NS(=O)(=O)c1cccc(c1)C(=O)N(Cc1ncc(C3CCC(C)(C)C3)cn1)[C@H](COC(C)C)CO2] | 1.57 | 685.305 | 686.5 | A |
| 1956 | 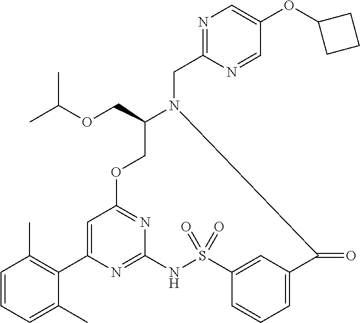 [SMILES: Cc1cccc(C)c1-c1cc2nc(n1)NS(=O)(=O)c1cccc(c1)C(=O)N(Cc1ncc(OC3CCC3)cn1)[C@H](COC(C)C)CO2] | 1.57 | 658.257 | 659.5 | A |
| 1957 | 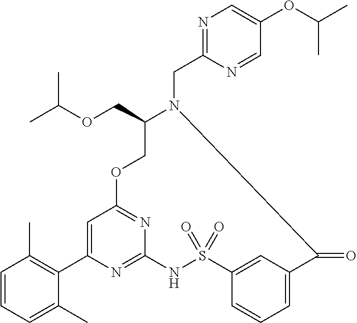 [SMILES: Cc1cccc(C)c1-c1cc2nc(n1)NS(=O)(=O)c1cccc(c1)C(=O)N(Cc1ncc(OC(C)C)cn1)[C@H](COC(C)C)CO2] | 1.51 | 646.257 | 647.4 | A |
| 1958 | 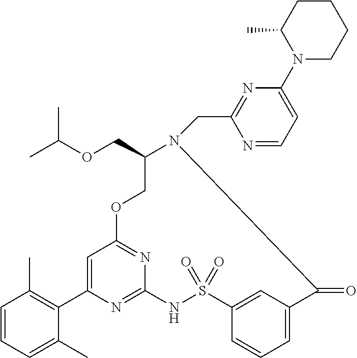 [SMILES: Cc1cccc(C)c1-c1cc2nc(n1)NS(=O)(=O)c1cccc(c1)C(=O)N(Cc1nccc(N3CCCC[C@H]3C)n1)[C@H](COC(C)C)CO2] | 1.74 | 685.305 | 686.3 | A |
| VRT- 2405654 1959 | 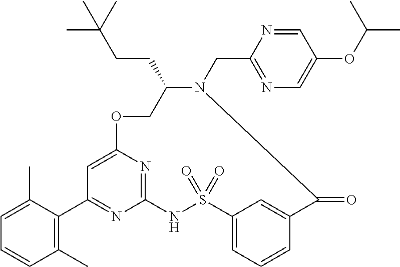 [SMILES: Cc1cccc(C)c1-c1cc2nc(n1)NS(=O)(=O)c1cccc(c1)C(=O)N(Cc1ncc(OC(C)C)cn1)[C@@H](CCC(C)(C)C)CO2] | 3.36 | 658.294 | 659.2 | 1D |
| 1960 | 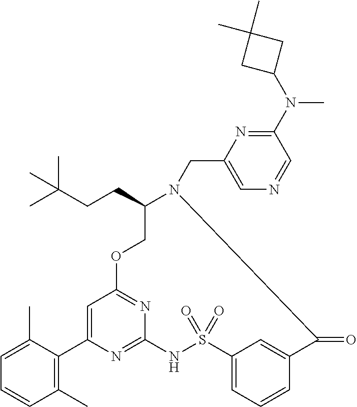 [SMILES: Cc1cccc(C)c1-c1cc2nc(n1)NS(=O)(=O)c1cccc(c1)C(=O)N(Cc1cncc(N(C)C3CC(C)(C)C3)n1)[C@H](CCC(C)(C)C)CO2] | 2.08 | 711.357 | 712.2 | A |
| 1961 | 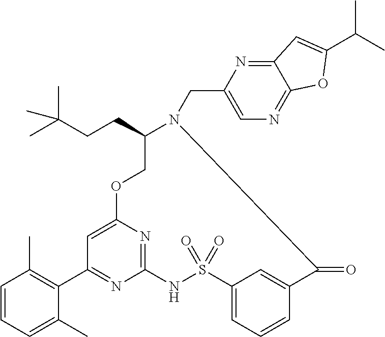 [SMILES: Cc1cccc(C)c1-c1cc2nc(n1)NS(=O)(=O)c1cccc(c1)C(=O)N(Cc1cnc3oc(C(C)C)cc3n1)[C@H](CCC(C)(C)C)CO2] | 2.19 | 682.294 | 683.68 | A |
| 1962 | 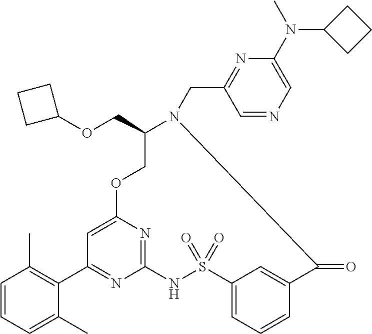 [SMILES: Cc1cccc(C)c1-c1cc2nc(n1)NS(=O)(=O)c1cccc(c1)C(=O)N(Cc1cncc(N(C)C3CCC3)n1)[C@H](COC1CCC1)CO2] | 1.76 | 683.289 | 684.2 | A |
| 1963 | 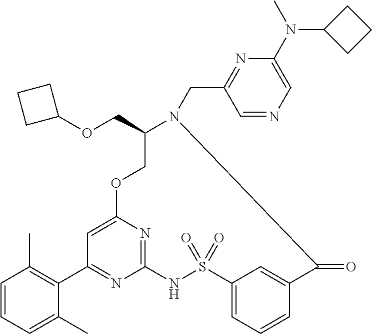 [SMILES: Cc1cccc(C)c1-c1cc2nc(n1)NS(=O)(=O)c1cccc(c1)C(=O)N(Cc1cncc(N(C)C3CCC3)n1)[C@H](COC1CCC1)CO2] | 1.78 | 683.289 | 684.2 | A |
| 1964 | 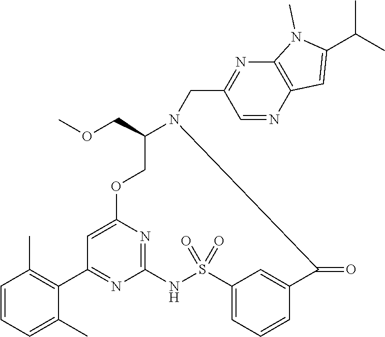 [SMILES: COC[C@@H]1COc2cc(-c3c(C)cccc3C)nc(n2)NS(=O)(=O)c2cccc(c2)C(=O)N1Cc1cnc2cc(C(C)C)n(C)c2n1] | 1.7 | 695.289 | 696.2 | A |
| 1965 | 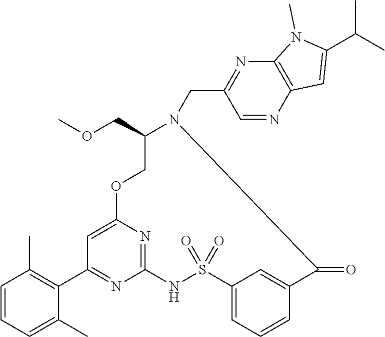 [SMILES: COC[C@@H]1COc2cc(-c3c(C)cccc3C)nc(n2)NS(=O)(=O)c2cccc(c2)C(=O)N1Cc1cnc2cc(C(C)C)n(C)c2n1] | 1.71 | 695.289 | 696.2 | A |
| 1966 | 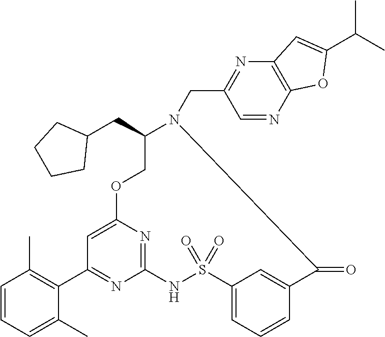 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCC1)CO2] | 2.08 | 680.278 | 681.2 | A |
| 1967 | 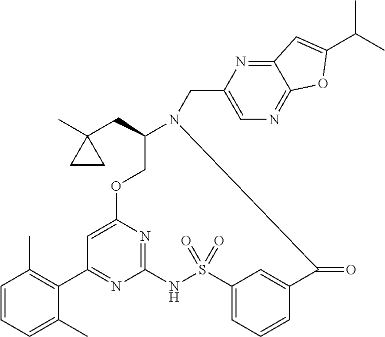 [SMILES: Cc1cccc(C)c1-c1cc2nc(n1)NS(=O)(=O)c1cccc(c1)C(=O)N(Cc1cnc3oc(C(C)C)cc3n1)[C@H](CC1(C)CC1)CO2] | 1.95 | 666.262 | 667.2 | A |
| 1968 | 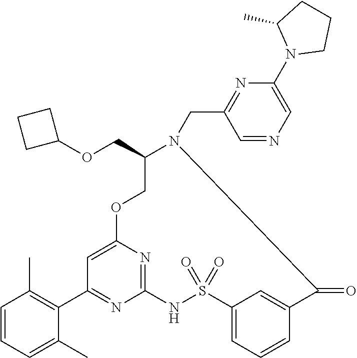 [SMILES: Cc1cccc(C)c1-c1cc2nc(n1)NS(=O)(=O)c1cccc(c1)C(=O)N(Cc1cncc(N3CCC[C@H]3C)n1)[C@H](COC1CCC1)CO2] | 1.72 | 683.289 | 684.2 | A |
| 1969 | 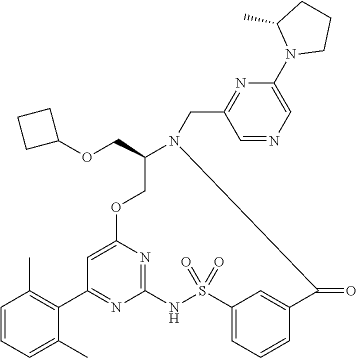 [SMILES: Cc1cccc(C)c1-c1cc2nc(n1)NS(=O)(=O)c1cccc(c1)C(=O)N(Cc1cncc(N3CCC[C@H]3C)n1)[C@H](COC1CCC1)CO2] | 1.72 | 683.289 | 684.2 | A |
| 1970 | 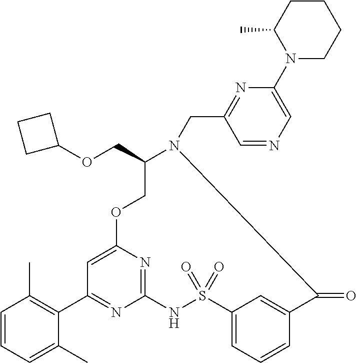 [SMILES: Cc1cccc(C)c1-c1cc2nc(n1)NS(=O)(=O)c1cccc(c1)C(=O)N(Cc1cncc(N3CCCC[C@H]3C)n1)[C@H](COC1CCC1)CO2] | 1.79 | 697.305 | 698.2 | A |
| 1971 | 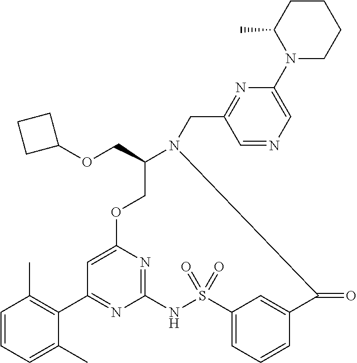 [SMILES: Cc1cccc(C)c1-c1cc2nc(n1)NS(=O)(=O)c1cccc(c1)C(=O)N(Cc1cncc(N3CCCC[C@H]3C)n1)[C@H](COC1CCC1)CO2] | 1.8 | 697.305 | 698.3 | A |
| 1972 | 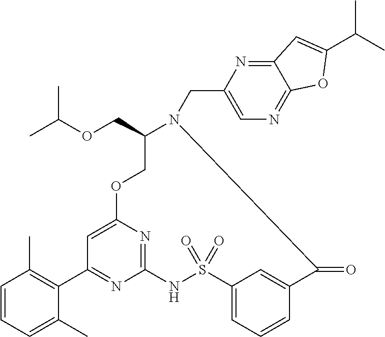 [SMILES: Cc1cccc(C)c1-c1cc2nc(n1)NS(=O)(=O)c1cccc(c1)C(=O)N(Cc1cnc3oc(C(C)C)cc3n1)[C@H](COC(C)C)CO2] | 2.06 | 670.257 | 671.43 | A |

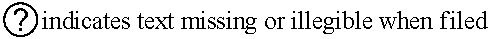

TABLE 15
NMR Data

| Compound Number | NMR |
|---|---|
| 1343 | ¹H NMR (400 MHz, Methanol-d4) 8 8.94 (s, 1H), 8.09 (d, J = |
| | 7.8 Hz, 1H), 7.73 (dt, J = 7.7, 1.4 Hz, 1H), 7.66 (t, J = 7.7 Hz, |
| | 1H), 7.34 - 7.15 (m, 3H), 7.10 (d, J = 7.6 Hz, 2H), 6.46 (d, J = |
| | 8.5 Hz, 1H), 6.20 (s, 1H), 5.51 (dd, J = 11.1, 4.6 Hz, 1H), 5.02 - |
| | 4.90 (m, 2H), 4.48 - 4.32 (m, 1H), 4.26 (d, J = 16.0 Hz, 1H), |
| | 3.58 - 3.42 (m, 5H), 2.30 (s, 3H), 1.31 - 1.18 (m, 10H), 1.10 (d, |
| | J = 6.7 Hz, 3H), 0.89 (dq, J = 8.0, 3.6 Hz, 3H), 0.62 (s, 9H). |
| 1346 | ¹H NMR (400 MHZ, DMSO-d6) 8 8.82 (s, 1H), 7.96 (d, J = 6.8 |
| | Hz, 1H), 7.70 (d, J = 5.9 Hz, 2H), 7.26 (t, J = 7.6 Hz, 2H), 7.12 |
| | (d, J = 7.7 Hz, 2H), 6.43 (s, 1H), 5.59 - 5.44 (m, 1H), 4.76 (d, |
| | J = 16.0 Hz, 1H), 4.20 (s, 2H), 4.04 (p, J = 8.1, 7.1 Hz, 1H), |
| | 2.27 (s, 3H), 2.14 - 1.88 (m, 9H), 1.69 (s, 3H), 1.42 (d, J = 15.0 |
| | Hz, 2H), 1.23 (d, J = 7.6 Hz, 1H), 1.17 (dt, J = 7.1, 4.0 Hz, 4H), 0.51 (s, 9H). |
| 1347 | ¹H NMR (400 MHZ, DMSO-d6) 8 8.68 (s, 1H), 8.01 - 7.85 (m, |
| | 1H), 7.70 (d, J = 4.7 Hz, 2H), 7.58 (s, 1H), 7.26 (t, J = 7.6 Hz, |
| | 1H), 7.13 (d, J = 7.7 Hz, 2H), 6.70 (d, J = 6.8 Hz, 2H), 6.44 (s, |
| | 1H), 5.34 (dt, J = 12.0, 6.6 Hz, 2H), 4.77 (dd, J = 16.2, 9.1 Hz, |
| | 1H), 4.46 (d, J = 16.4 Hz, 2H), 4.26 (q, J = 10.7 Hz, 2H), 4.08 |
| | (q, J = 8.5, 6.4 Hz, 1H), 3.95 (tdd, J = 8.0, 4.7, 2.6 Hz, 1H), |
| | 3.75 - 3.72 (m, 1H), 3.64 - 3.52 (m, 1H), 2.95 (d, J = 4.0 Hz, |
| | 3H), 2.34 - 2.24 (m, 1H), 2.01 (s, 5H), 1.89 - 1.71 (m, 3H), 0.54 (s, 9H). |
| 1348 | ¹H NMR (400 MHZ, DMSO-d6) 8 12.95 (s, 1H), 8.65 (d, J = |
| | 7.4 Hz, 1H), 7.95 (s, 1H), 7.86 (d, J = 11.5 Hz, 1H), 7.68 (d, J = |
| | 4.2 Hz, 2H), 7.24 (d, J = 7.8 Hz, 1H), 7.12 (d, J = 7.7 Hz, |
| | 2H), 6.82 (dd, J = 25.3, 8.3 Hz, 1H), 6.44 (s, 1H), 5.38 (dd, J = |
| | 70.0, 9.4 Hz, 1H), 4.64 (dd, J = 35.3, 15.7 Hz, 1H), 4.44 - 4.17 |
| | (m, 3H), 4.06 (s, 1H), 2.21 - 1.87 (m, 9H), 1.83 - 1.51 (m, 10H), |
| | 1.39 (dd, J = 15.0, 8.7 Hz, 2H), 0.54 (d, J = 6.6 Hz, 9H). |
| 1349 | ¹H NMR (400 MHZ, DMSO-d6) 8 12.85 (s, 1H), 8.67 (d, J = |
| | 2.2 Hz, 1H), 8.05 (d, J = 2.2 Hz, 1H), 7.95 (d, J = 6.7 Hz, 1H), |
| | 7.90 (s, 1H), 7.69 (d, J = 6.6 Hz, 2H), 7.26 (t, J = 7.6 Hz, 1H), |
| | 7.12 (d, J = 7.6 Hz, 2H), 6.44 (s, 1H), 5.38 (td, J = 10.8, 4.3 |
| | Hz, 1H), 5.33 - 5.21 (m, 1H), 4.74 (dd, J = 16.0, 6.5 Hz, 1H), |
| | 4.47 (dd, J = 16.0, 5.9 Hz, 1H), 4.31 (d, J = 8.8 Hz, 1H), 4.11 - |
| | 4.01 (m, 1H), 3.95 (tt, J = 8.6, 4.6 Hz, 1H), 3.79 (dd, J = 5.2, |
| | 2.5 Hz, 1H), 3.76 - 3.73 (m, 1H), 3.63 (p, J = 8.3 Hz, 1H), 2.99 |
| | (d, J = 2.8 Hz, 3H), 2.40 - 1.68 (m, 10H), 0.56 (s, 9H). |
| 1354 | ¹H NMR (400 MHZ, DMSO) § 13.08 (s, 1H), 8.69 (s, 1H), 7.96 |
| | (s, 1H), 7.70 (s, 2H), 7.26 (t, J = 7.6 Hz, 1H), 7.12 (d, J = 7.7 |
| | Hz, 2H), 6.62 (s, 2H), 6.44 (s, 1H), 5.36 (s, 1H), 4.75 (s, 1H), |
| | 4.42 (s, 1H), 4.26 (s, 1H), 4.08 (s, 1H), 3.79 (s, 4H), 3.09 (s, |
| | 2H), 1.95 (d, J = 35.1 Hz, 8H), 1.77 (dd, J = 15.1, 9.0 Hz, 1H), |
| | 1.40 (d, J = 15.0 Hz, 1H), 0.93 (s, 3H), 0.53 (s, 9H), 0.46 (s, 2H). |
| 1355 | ¹H NMR (400 MHZ, DMSO) 8 12.96 (s, 1H), 8.65 (s, 1H), 7.96 |
| | (s, 1H), 7.69 (s, 2H), 7.49 (s, 1H), 7.26 (t, J = 7.6 Hz, 1H), 7.12 |
| | (d, J = 7.7 Hz, 2H), 6.61 (s, 2H), 6.44 (s, 1H), 5.34 (d, J = 10.4 |
| | Hz, 1H), 4.91 (s, 1H), 4.77 - 4.67 (m, 1H), 4.39 (s, 1H), 4.27 (t, |
| | J = 11.4 Hz, 1H), 4.07 (d, J = 8.3 Hz, 1H), 2.90 (s, 3H), 2.12 - |
| | 1.90 (m, 6H), 1.84 (s, 2H), 1.77 (dd, J = 15.3, 9.1 Hz, 2H), 1.66 |
| | (s, 2H), 1.53 (s, 3H), 1.39 (d, J = 15.1 Hz, 1H), 0.54 (s, 9H). |
| 1356 | ¹H NMR (400 MHZ, DMSO-d6) § 12.78 (br. s., 1H), 8.78 (s, |
| | 1H), 8.17 (d, J = 6.1 Hz, 1H), 7.95 (d, J = 6.4 Hz, 1H), 7.79 - |
| | 7.58 (m, 2H), 7.34 - 7.19 (m, 1H), 7.18 - 7.01 (m, 2H), 6.58 (d, |
| | J = 5.9 Hz, 1H), 6.42 (br. s., 1H), 5.62 - 5.23 (m, 2H), 4.68 (d, |
| | J = 16.6 Hz, 1H), 4.46 (d, J = 16.9 Hz, 1H), 4.25 - 4.13 (m, |
| | 1H), 4.12 - 4.03 (m, 1H), 3.95 (td, J = 8.4, 4.6 Hz, 1H), 3.88 - |
| | 3.73 (m, 2H), 3.60 (q, J = 8.2 Hz, 1H), 2.94 (s, 3H), 2.28 - 1.88 |
| | (m, 7H), 1.84 - 1.66 (m, 2H), 1.39 (d, J = 14.4 Hz, 1H), 0.56 (s, 9H). |
| 1357 | ¹H NMR (400 MHZ, DMSO-d6) 8 12.74 (br. s., 1H), 8.79 (s, |
| | 1H), 8.12 (d, J = 6.1 Hz, 1H), 7.94 (d, J = 6.4 Hz, 1H), 7.73 - |
| | 7.62 (m, 2H), 7.30 - 7.21 (m, 1H), 7.17 - 7.07 (m, 2H), 6.49 (d, |
| | J = 6.1 Hz, 1H), 6.39 (br. s., 1H), 5.44 (dd, J = 10.5, 3.9 Hz, |
| | 1H), 4.67 (d, J = 16.6 Hz, 1H), 4.44 (d, J = 16.9 Hz, 1H), 4.27 - |
| | 4.02 (m, 2H), 3.79 - 3.49 (m, 2H), 3.04 (br. s., 3H), 2.20 - 1.84 |
| | (m, 6H), 1.73 (dd, J = 15.2, 8.8 Hz, 1H), 1.39 (d, J = 14.7 Hz, |
| | 1H), 1.07 (t, J = 7.1 Hz, 3H), 0.56 (s, 9H). |
| 1358 | ¹H NMR (400 MHz, DMSO-d6) 8 13.01 (br. s, 1H), 8.50 (br. s, |
| | 1H), 7.97 - 7.87 (m, 1H), 7.65 (br. s, 2H), 7.52 (s, 1H), 7.30 - |
| | 7.20 (m, 1H), 7.17 - 7.06 (m, 2H), 6.49 - 6.25 (m, 1H), 5.23 (dd, |
| | J = 10.3, 4.4 Hz, 1H), 4.56 (d, J = 15.2 Hz, 1H), 4.37 - 4.25 |
| | (m, 1H), 4.18 (d, J = 15.4 Hz, 1H), 3.94 - 3.83 (m, 1H), 3.72 - |
| | 3.64 (m, 4H), 3.42 - 3.36 (m, 4H), 2.24 - 1.82 (m, 7H), 1.38 (d, |
| | J = 15.2 Hz, 1H), 0.52 (s, 9H). |
| 1360 | ¹H NMR (400 MHZ, DMSO-d6) 8 13.06 (s, 1H), 8.70 (s, 1H), |
| | 8.59 (s, 2H), 7.95 (s, 1H), 7.67 (s, 2H), 7.26 (s, 1H), 7.13 (s, |
| | 2H), 6.41 (s, 1H), 5.76 (s, 1H), 5.40 (d, J = 9.8 Hz, 1H), 4.77 = |
| | (dd, J = 79.2, 16.5 Hz, 2H), 4.50 (d, J = 5.8 Hz, 2H), 4.31 (d, J |
| | 5.8 Hz, 2H), 4.24 (s, 2H), 4.04 (s, 1H), 2.03 (d, J = 68.4 Hz, |
| | 6H), 1.79 (dd, J = 15.1, 8.7 Hz, 1H), 1.37 (s, 3H), 1.24 (s, 1H), |
| | 0.57 (s, 9H). |
| 1362 | ¹H NMR (400 MHZ, DMSO-d6) 8 12.85 (s, 1H), 8.79 (s, 1H), |
| | 8.51 (s, 1H), 7.94 (d, J = 7.6 Hz, 1H), 7.72 - 7.59 (m, 2H), 7.26 |
| | (t, J = 7.6 Hz, 1H), 7.12 (d, J = 7.7 Hz, 2H), 6.41 (s, 1H), 5.48 |
| | (dd, J = 10.9, 4.3 Hz, 1H), 4.87 (d, J = 16.8 Hz, 1H), 4.73 (s, |
| | 2H), 4.68 (d, J = 16.9 Hz, 1H), 4.14 (t, J = 11.1 Hz, 1H), 4.02 - |
| | 3.97 (m, 3H), 2.98 - 2.89 (m, 1H), 2.87 - 2.79 (m, 1H), 2.15 - |
| | 1.86 (m, 6H), 1.83 - 1.74 (m, 1H), 1.42 (d, J = 14.7 Hz, 1H), |
| | 0.59 (s, 9H). |
| 1365 | ¹H NMR (400 MHZ, DMSO-d6) 8 13.08 (s, 1H), 9.07 (d, J = |
| | 2.0 Hz, 2H), 8.72 (s, 1H), 7.94 (s, 1H), 7.66 (s, 2H), 7.25 (s, |
| | 1H), 7.12 (s, 2H), 6.41 (s, 1H), 5.41 (s, 1H), 4.88 (d, J = 16.7 |
| | Hz, 1H), 4.74 (t, J = 15.6 Hz, 1H), 4.20 (s, 1H), 4.04 (s, 1H), |
| | 3.66 (d, J = 2.0 Hz, 2H), 2.03 (m, 6H), 1.77 (s, 2H), 1.41 (d, J = |
| | 15.0 Hz, 2H), 1.28 - 1.09 (m, 6H), 0.57 (s, 9H). |
| 1366 | ¹H NMR (400 MHZ, DMSO-d6) 8 12.93 (s, 1H), 8.71 (s, 1H), |
| | 7.95 (d, J = 7.0 Hz, 1H), 7.81 (d, J = 12.7 Hz, 2H), 7.68 (d, J = |
| | 7.5 Hz, 2H), 7.26 (t, J = 7.6 Hz, 1H), 7.12 (d, J = 7.7 Hz, |
| | 2H), 6.43 (s, 1H), 5.51 (dd, J = 10.9, 4.3 Hz, 1H), 4.70 (d, J = |
| | 15.9 Hz, 1H), 4.46 (d, J = 16.0 Hz, 1H), 4.37 - 4.26 (m, 2H), |
| | 4.07 - 3.98 (m, 1H), 3.61 - 3.56 (m, 2H), 3.42 - 3.35 (m, 1H), |
| | 2.12 - 1.87 (m, 8H), 1.78 - 1.68 (m, 2H), 1.43 (d, J = 14.7 Hz, |
| | 1H), 1.22 (d, J = 6.2 Hz, 3H), 0.57 (s, 9H). |
| 1367 | ¹H NMR (400 MHZ, DMSO-d6) 8 13.07 (s, 1H), 8.61 (s, 1H), |
| | 8.14 (s, 1H), 7.96 (s, 1H), 7.82 (s, 1H), 7.69 (s, 2H), 7.26 (t, J = |
| | 7.6 Hz, 1H), 7.12 (d, J = 7.6 Hz, 2H), 6.45 (s, 1H), 5.27 (dd, |
| | J = 10.7, 4.4 Hz, 1H), 4.66 (d, J = 15.6 Hz, 1H), 4.44 (d, J = |
| | 15.7 Hz, 1H), 4.34 (t, J = 11.2 Hz, 1H), 4.11 - 4.04 (m, 1H), |
| | 3.89 (dt, J = 14.5, 5.3 Hz, 1H), 3.73 (dt, J = 14.5, 5.8 Hz, 1H), |
| | 3.55 - 3.48 (m, 3H), 3.45 (s, 1H), 3.11 (s, 3H), 1.95 (s, 6H), 1.76 |
| | (dd, J = 15.3, 8.9 Hz, 1H), 1.39 (d, J = 15.0 Hz, 1H), 0.92 (s, 9H), 0.53 (s, 9H). |
| 1368 | ¹H NMR (400 MHZ, Chloroform-d) 8 8.84 (s, 1H), 8.22 (d, J = |
| | 2.3 Hz, 1H), 8.06 (dt, J = 7.4, 1.6 Hz, 1H), 7.82 - 7.52 (m, 2H), |
| | 7.35 - 7.22 (m, 2H), 7.15 (d, J = 7.6 Hz, 2H), 6.28 (s, 1H), 5.78 - |
| | 5.41 (m, 1H), 5.14 (d, J = 15.8 Hz, 1H), 4.77 - 4.56 (m, 1H), |
| | 4.44 (d, J = 15.9 Hz, 1H), 4.16 (t, J = 5.0 Hz, 2H), 3.95 (dd, J = |
| | 10.9, 5.7 Hz, 2H), 3.62 (td, J = 8.8, 4.4 Hz, 2H), 2.41 - 1.93 |
| | (m, 8H), 1.88 (dd, J = 15.4, 8.1 Hz, 1H), 1.75 (dtd, J = 12.8, |
| | 8.5, 3.9 Hz, 2H), 1.59 (d, J = 15.2 Hz, 1H), 0.69 (s, 9H). |
| 1369 | ¹H NMR (400 MHZ, DMSO-d6) 8 13.08 (s, 1H), 8.69 (s, 1H), |
| | 8.45 (d, J = 11.8 Hz, 2H), 7.95 (s, 1H), 7.80 - 7.42 (m, 2H), |
| | 7.25 (d, J = 7.9 Hz, 1H), 7.12 (d, J = 7.5 Hz, 2H), 6.42 (s, 1H), |
| | 5.76 (s, 1H), 5.37 (d, J = 8.4 Hz, 1H), 4.76 (dd, J = 73.6, 16.6 |
| | Hz, 2H), 4.57 (p, J = 6.8 Hz, 1H), 4.38 - 3.79 (m, 3H), 3.62 (p, |
| | J = 6.9 Hz, 1H), 3.16 (d, J = 3.9 Hz, 3H), 3.02 - 2.76 (m, 2H), |
| | 2.22 - 1.86 (m, 6H), 1.77 (dd, J = 15.1, 8.8 Hz, 1H), 1.39 (d, J = |
| | 15.0 Hz, 1H), 0.56 (s, 9H). |
| 1370 | ¹H NMR (400 MHZ, DMSO) & 13.05 (s, 1H), 8.53 (s, 1H), 8.08 |
| | (d, J = 2.4 Hz, 1H), 7.92 (s, 1H), 7.66 (s, 2H), 7.38 - 7.21 (m, |
| | 2H), 7.13 (d, J = 7.7 Hz, 2H), 6.39 (s, 1H), 5.32 (d, J = 8.3 Hz, |
| | 1H), 4.93 (d, J = 15.4 Hz, 1H), 4.82 (p, J = 7.1 Hz, 1H), 4.42 |
| | (d, J = 15.7 Hz, 1H), 4.24 (t, J = 11.1 Hz, 1H), 4.00 (d, J = 9.2 |
| | Hz, 1H), 2.16 - 1.92 (m, 9H), 1.78 (ddd, J = 13.5, 10.3, 2.8 Hz, |
| | 2H), 1.64 (tt, J = 10.3, 8.1 Hz, 1H), 1.40 (s, 1H), 1.22 (ddd, J = |
| | 13.5, 9.3, 3.0 Hz, 2H), 0.76 (d, J = 6.6 Hz, 3H), 0.27 (d, J = |
| | 6.3 Hz, 3H). |
| 1299 | ¹H NMR (400 MHZ, DMSO) 8 13.07 (s, 1H), 8.59 (s, 1H), 8.07 |
| | (d, J = 2.4 Hz, 1H), 7.94 (s, 1H), 7.66 (s, 2H), 7.34 - 7.22 (m, |
| | 2H), 7.13 (d, J = 7.2 Hz, 2H), 6.43 (s, 1H), 5.35 (d, J = 8.2 Hz, |
| | 1H), 4.90 (d, J = 15.5 Hz, 1H), 4.82 (p, J = 7.1 Hz, 1H), 4.44 |
| | (d, J = 15.8 Hz, 1H), 4.24 (t, J = 11.2 Hz, 1H), 4.07 - 3.98 (m, |
| | 1H), 2.47 - 2.42 (m, 2H), 2.20 - 1.91 (m, 8H), 1.85 - 1.74 (m, |
| | 2H), 1.70 - 1.56 (m, 1H), 1.41 (d, J = 15.0 Hz, 1H), 0.56 (s, |
| | 9H). |
| 1371 | ¹H NMR (400 MHZ, DMSO) 8 12.97 (s, 1H), 8.59 (s, 1H), 7.96 |
| | (s, 1H), 7.70 (d, J = 4.8 Hz, 2H), 7.54 (s, 1H), 7.26 (t, J = 7.7 |
| | Hz, 1H), 7.13 (d, J = 7.7 Hz, 2H), 6.67 (d, J = 9.3 Hz, 1H), 6.45 |
| | (s, 1H), 5.31 (dd, J = 10.8, 4.3 Hz, 1H), 4.76 (d, J = 15.3 Hz, |
| | 1H), 4.66 - 4.62 (m, 1H), 4.41 - 4.34 (m, 1H), 4.28 (t, J = 11.1 |
| | Hz, 1H), 4.09 - 4.00 (m, 2H), 3.91 - 3.86 (m, 8H), 3.31 (dt, J = |
| | 24.6, 11.6 Hz, 2H), 2.91 (s, 3H), 1.79 (dd, J = 15.2, 8.8 Hz, |
| | 3H), 1.62 - 1.52 (m, 2H), 1.40 (d, J = 15.0 Hz, 1H), 0.55 (s, |
| | 9H). |
| 1374 | ¹H NMR (400 MHZ, DMSO-d6) 8 8.69 (s, 1H), 8.53 (s, 2H), |
| | 7.95 (s, 1H), 7.67 (s, 2H), 7.25 (d, J = 7.6 Hz, 1H), 7.13 (d, J = |
| | 7.6 Hz, 2H), 6.43 (s, 1H), 5.43 - 5.30 (m, 1H), 4.85 (d, J = 16.5 |
| | Hz, 1H), 4.70 - 4.55 (m, 2H), 4.19 (d, J = 11.2 Hz, 1H), 4.05 |
| | (s, 1H), 3.75 - 3.71 (m, 1H), 1.99 (d, J = 66.2 Hz, 6H), 1.78 |
| | (dd, J = 15.2, 8.9 Hz, 1H), 1.71 - 1.57 (m, 2H), 1.40 (d, J = |
| | 15.0 Hz, 1H), 1.26 (d, J = 6.0 Hz, 3H), 0.93 (t, J = 7.4 Hz, 3H), |
| | 0.56 (s, 9H). |
| 1375 | ¹H NMR (400 MHZ, DMSO-d6) 8 8.69 (s, 1H), 8.53 (s, 2H), |
| | 7.95 (s, 1H), 7.67 (s, 2H), 7.26 (t, J = 7.5 Hz, 1H), 7.13 (d, J = |
| | 7.7 Hz, 2H), 6.43 (s, 1H), 5.39 (d, J = 9.2 Hz, 1H), 4.85 (d, J = |
| | 16.5 Hz, 1H), 4.72 - 4.58 (m, 2H), 4.20 (d, J = 10.9 Hz, 1H), |
| | 4.05 (s, 1H), 1.93 (d, J = 15.7 Hz, 7H), 1.83 - 1.75 (m, 1H), |
| | 1.71 - 1.56 (m, 2H), 1.40 (d, J = 15.0 Hz, 1H), 1.26 (d, J = 6.0 |
| | Hz, 3H), 0.93 (t, J = 7.4 Hz, 3H), 0.56 (s, 9H). |
| 1376 | ¹H NMR (400 MHZ, DMSO) 8 8.70 (s, 1H), 7.92 (s, 1H), 7.64 |
| | (t, J = 9.0 Hz, 3H), 7.23 (s, 1H), 7.10 (d, J = 7.5 Hz, 2H), 6.74 |
| | (dd, J = 8.9, 3.0 Hz, 1H), 6.37 (s, 1H), 5.45 (d, J = 9.3 Hz, 1H), |
| | 4.89 (dd, J = 16.2, 1.8 Hz, 1H), 4.44 (d, J = 16.1 Hz, 1H), 4.26 |
| | (s, 1H), 4.15 (dd, J = 10.2, 6.1 Hz, 1H), 4.11 (s, 1H), 4.08 (dd, |
| | J = 10.2, 6.3 Hz, 1H), 3.29 (d, J = 7.1 Hz, 1H), 2.69 (d, J = 0.8 |
| | Hz, 1H), 2.18 (t, J = 8.1 Hz, 1H), 2.04 (hept, J = 6.4 Hz, 1H), |
| | 1.96 - 1.84 (m, 1H), 1.75 (dd, J = 15.0, 8.9 Hz, 1H), 1.43 (d, J = |
| | 14.9 Hz, 1H), 1.24 (s, 2H), 0.92 (dd, J = 6.8, 2.8 Hz, 6H), |
| | 0.57 (s, 9H). |
| 1300 | ¹H NMR (400 MHZ, DMSO) 8 7.95 (d, J = 6.7 Hz, 1H), 7.68 |
| | (s, 2H), 7.43 (t, J = 9.3 Hz, 1H), 7.27 (t, J = 7.6 Hz, 1H), 7.13 |
| | (d, J = 7.7 Hz, 2H), 6.54 (dd, J = 9.2, 2.6 Hz, 1H), 6.46 (s, 1H), |
| | 5.56 - 5.47 (m, 1H), 5.05 (p, J = 8.2 Hz, 1H), 4.34 (dd, J = 13.9, |
| | 7.4 Hz, 2H), 4.10 (s, 1H), 3.04 (s, 3H), 2.47 - 2.32 (m, 2H), 2.29 - |
| | 2.09 (m, 2H), 2.01 (s, 7H), 1.77 (dd, J = 15.2, 8.8 Hz, 1H), |
| | 1.43 (d, J = 14.9 Hz, 1H), 1.24 (s, 3H), 0.57 (s, 9H), 0.37 (s, |
| | 2H), 0.42 - 0.31 (m, 1H). |
| 1301 | ¹H NMR (400 MHZ, DMSO-d6) 8 13.08 (s, 1H), 8.70 (s, 1H), |
| | 8.53 (s, 2H), 7.95 (s, 1H), 7.67 (s, 2H), 7.30 - 7.20 (m, 1H), 7.12 |
| | (d, J = 7.6 Hz, 2H), 6.42 (s, 1H), 5.43 - 5.31 (m, 1H), 4.85 (d, |
| | J = 16.5 Hz, 1H), 4.65 (d, J = 16.6 Hz, 1H), 4.47 (p, J = 6.1 |
| | Hz, 1H), 4.20 (t, J = 11.2 Hz, 1H), 4.10 - 4.01 (m, 1H), 2.25 - |
| | 1.90 (m, 6H), 1.91 - 1.86 (m, 1H), 1.77 (dd, J = 15.1, 8.8 Hz, |
| | 1H), 1.40 (d, J = 15.0 Hz, 1H), 1.20 (d, J = 6.1 Hz, 3H), 0.94 |
| | (dd, J = 13.7, 6.8 Hz, 6H), 0.56 (s, 9H). |
| 1377 | ¹H NMR (400 MHZ, DMSO-d6) & 13.08 (s, 1H), 8.70 (s, 1H), |
| | 8.53 (s, 2H), 7.95 (s, 1H), 7.67 (s, 2H), 7.33 - 7.20 (m, 1H), 7.18 - |
| | 7.05 (m, 2H), 6.42 (s, 1H), 5.40 (d, J = 9.5 Hz, 1H), 4.84 (d, |
| | J = 16.5 Hz, 1H), 4.66 (d, J = 16.5 Hz, 1H), 4.46 (p, J = 6.1 |
| | Hz, 1H), 4.21 (t, J = 11.2 Hz, 1H), 4.09 - 3.99 (m, 1H), 2.22 - |
| | 1.90 (m, 6H), 1.91 - 1.86 (m, 1H), 1.77 (dd, J = 15.2, 8.8 Hz, |
| | 1H), 1.40 (d, J = 14.9 Hz, 1H), 1.21 (d, J = 6.1 Hz, 3H), 0.94 |
| | (dd, J = 12.1, 6.8 Hz, 6H), 0.57 (s, 9H). |
| 1302 | ¹H NMR (400 MHZ, DMSO-d6) § 13.06 (br. s., 1H), 8.81 (br. |
| | s., 1H), 8.66 (d, J = 5.1 Hz, 1H), 7.94 (br. s., 1H), 7.67 (br. s., |
| | 2H), 7.31 - 7.21 (m, 2H), 7.18 - 7.07 (m, 2H), 6.40 (br. s., 1H), |
| | 5.51 (d, J = 6.8 Hz, 1H), 4.87 (d, J = 17.1 Hz, 1H), 4.70 (d, J = |
| | 17.1 Hz, 1H), 4.18 (t, J = 11.1 Hz, 1H), 4.06 - 3.94 (m, 1H), |
| | 2.78 (q, J = 7.5 Hz, 2H), 2.26 - 1.85 (m, 6H), 1.77 (dd, J = 15.4, |
| | 9.0 Hz, 1H), 1.42 (d, J = 14.4 Hz, 1H), 1.26 (t, J = 7.6 Hz, 3H), |
| | 0.59 (s, 9H). |
| 1378 | ¹H NMR (400 MHZ, DMSO-d6) 8 13.12 (br. s, 1H), 8.50 (br. s, |
| | 1H), 8.00 - 7.85 (m, 1H), 7.65 (br. s, 2H), 7.44 (s, 1H), 7.32 - |
| | 7.04 (m, 3H), 6.40 (br. s, 1H), 5.28 - 5.15 (m, 1H), 4.53 (d, J = |
| | 15.4 Hz, 1H), 4.39 - 4.25 (m, 1H), 4.17 (d, J = 15.4 Hz, 1H), |
| | 13833.96 - 3.79 (m, 1H), 3.45 - 3.37 (m, 4H), 2.21 - 1.81 (m, |
| | 7H), 1.58 - 1.53 (m, 6H), 1.37 (d, J = 14.7 Hz, 1H), 0.51 (s, |
| | 9H). |
| 1379 | ¹H NMR (400 MHZ, DMSO-d6) 8 13.04 (s, 1H), 8.69 (s, 1H), |
| | 8.23 (s, 2H), 7.95 (s, 1H), 7.67 (s, 2H), 7.25 (d, J = 7.7 Hz, 1H), |
| | 7.12 (d, J = 7.6 Hz, 2H), 6.41 (s, 1H), 5.38 (d, J = 7.8 Hz, 1H), |
| | 4.85 (d, J = 16.3 Hz, 1H), 4.59 (d, J = 16.4 Hz, 1H), 4.13 (t, J = |
| | 11.1 Hz, 1H), 4.04 (d, J = 8.6 Hz, 1H), 3.80 (t, J = 13.2 Hz, |
| | 2H), 3.58 (t, J = 7.2 Hz, 2H), 2.58 (s, 1H), 2.02 (d, J = 63.5 Hz, |
| | 7H), 1.79 (dd, J = 15.1, 8.8 Hz, 1H), 1.39 (d, J = 15.0 Hz, 1H), |
| | 0.57 (s, 9H). |
| 1380 | ¹H NMR (400 MHZ, DMSO-d6) 8 13.08 (s, 1H), 8.69 (s, 1H), |
| | 8.56 (s, 1H), 7.95 (s, 1H), 7.67 (s, 2H), 7.26 (t, J = 7.6 Hz, 1H), |
| | 7.13 (d, J = 7.6 Hz, 2H), 6.39 (d, J = 29.4 Hz, 1H), 5.38 (d, J = |
| | 5.6 Hz, 1H), 4.86 (d, J = 16.5 Hz, 1H), 4.71 - 4.61 (m, 1H), |
| | 4.32 - 4.26 (m, 1H), 4.20 (t, J = 11.1 Hz, 1H), 4.05 (d, J = 8.8 |
| | Hz, 1H), 3.65 - 3.50 (m, 4H), 3.30 (s, 3H), 2.29 - 1.86 (m, 6H), |
| | 1.78 (dd, J = 15.2, 8.8 Hz, 1H), 1.40 (d, J = 15.0 Hz, 1H), 0.57 |
| | (s, 9H). |
| 1383 | ¹H NMR (400 MHZ, DMSO-d6) 8 12.85 (s, 1H), 8.66 (s, 1H), |
| | 7.93 (d, J = 6.5 Hz, 1H), 7.85 (d, J = 4.2 Hz, 2H), 7.67 (d, J = |
| | 5.7 Hz, 2H), 7.25 (t, J = 7.6 Hz, 1H), 7.12 (d, J = 7.6 Hz, 2H), |
| | 6.39 (s, 1H), 5.37 (dd, J = 11.1, 4.2 Hz, 1H), 4.72 (d, J = 15.6 |
| | Hz, 1H), 4.39 (d, J = 15.6 Hz, 1H), 4.27 (s, 1H), 4.11 - 3.99 (m, |
| | 2H), 3.64 (td, J = 9.2, 7.6, 4.0 Hz, 1H), 3.52 (dt, J = 10.8, |
| | 7.6Hz, 1H), 2.28 - 2.16 (m, 1H), 2.14 - 1.70 (m, 11H), 1.42 (d, |
| | J = 15.0 Hz, 1H), 0.85 (d, J = 6.9 Hz, 3H), 0.78 (d, J = 6.7 Hz, |
| | 3H), 0.56 (s, 9H). |
| 1384 | ¹H NMR (400 MHZ, DMSO-d6) § 12.85 (s, 1H), 8.64 (s, 1H), |
| | 7.93 (d, J = 7.2 Hz, 1H), 7.84 (s, 2H), 7.66 (s, 2H), 7.25 (t, J = |
| | 7.6 Hz, 1H), 7.12 (d, J = 7.7 Hz, 2H), 6.41 (s, 1H), 5.49 (d, J = |
| | 7.0 Hz, 1H), 4.66 (d, J = 15.5 Hz, 1H), 4.42 (d, J = 15.5 Hz, |
| | 1H), 4.34 (s, 1H), 4.21 (d, J = 5.3 Hz, 1H), 4.02 (s, 1H), 3.63 - |
| | 3.54 (m, 1H), 3.54 - 3.47 (m, 1H), 2.37 (q, J = 6.4 Hz, 1H), |
| | 2.19 - 1.83 (m, 10H), 1.74 (dd, J = 15.2, 9.0 Hz, 1H), 1.43 (d, |
| | J = 14.9 Hz, 1H), 0.88 (d, J = 6.9 Hz, 3H), 0.75 (d, J = 6.8 Hz, |
| | 3H), 0.58 (s, 9H). |
| 1387 | ¹H NMR (400 MHZ, DMSO) 8 13.04 (s, 1H), 8.64 (s, 1H), 8.03 |
| | (s, 1H), 7.95 (d, J = 6.2 Hz, 1H), 7.84 (s, 1H), 7.68 (s, 2H), 7.26 |
| | (t, J = 7.6 Hz, 1H), 7.13 (d, J = 7.6 Hz, 2H), 6.45 (s, 1H), 5.37 |
| | (dd, J = 10.9, 4.2 Hz, 1H), 4.89 (t, J = 8.1 Hz, 1H), 4.71 (d, J = |
| | 15.8 Hz, 1H), 4.44 (d, J = 15.8 Hz, 1H), 4.32 (t, J = 11.2 Hz, |
| | 1H), 4.10 - 4.01 (m, 1H), 2.94 (s, 3H), 2.20 - 1.90 (m, 6H), 1.89 - |
| | 1.81 (m, 2H), 1.76 (dd, J = 15.2, 9.0 Hz, 1H), 1.71 - 1.63 (m, |
| | 2H), 1.60 - 1.49 (m, 4H), 1.41 (d, J = 14.9 Hz, 1H), 0.56 (s, |
| | 9H). |
| 1388 | ¹H NMR (400 MHZ, Methanol-d4) 8 8.89 (s, 1H), 8.05 (d, J = |
| | 7.8, 1.5 Hz, 1H), 7.76 (d, J = 7.6, 1.5 Hz, 1H), 7.69 (t, J = 7.7 |
| | Hz, 1H), 7.43 (dd, J = 8.5, 7.2 Hz, 1H), 7.25 (t, J = 7.6 Hz, 1H), |
| | 7.12 (d, J = 7.6 Hz, 2H), 6.64 (d, J = 7.2 Hz, 1H), 6.54 (d, J = |
| | 8.5 Hz, 1H), 6.24 (s, 1H), 5.41 (dd, J = 11.0, 4.3 Hz, 1H), 5.07 |
| | (d, J = 15.7 Hz, 1H), 4.32 (d, J = 15.7 Hz, 1H), 4.25 - 4.21 (m, |
| | 1H), 4.10 (q, J = 7.1 Hz, 1H), 4.01 (t, J = 11.3 Hz, 1H), 3.82 - |
| | 3.71 (m, 1H), 3.71 - 3.61 (m, 1H), 2.13 - 2.02 (m, 4H), 2.01 (s, |
| | 1H), 1.98 - 1.84 (m, 4H), 1.52 (d, J = 15.2, 1.6 Hz, 1H), 1.29 |
| | (s, 1H), 1.24 (t, J = 7.1 Hz, 1H), 0.90 (s, 9H), 0.64 (s, 9H). |
| 1389 | ¹H NMR (400 MHZ, Methanol-d4) 8 8.79 (d, J = 1.8 Hz, 1H), |
| | 8.04 (dt, J = 7.6, 1.6 Hz, 1H), 7.74 (dt, J = 7.7, 1.5 Hz, 1H), |
| | 7.68 (t, J = 7.7 Hz, 1H), 7.42 (t, J = 8.5, 7.2 Hz, 1H), 7.26 (t, J = |
| | 7.6 Hz, 1H), 7.13 (s, 2H), 6.66 (d, J = 7.2 Hz, 1H), 6.53 (d, J = |
| | 8.5 Hz, 1H), 6.27 (s, 1H), 5.60 (dd, J = 10.5, 3.8 Hz, 1H), |
| | 4.39 - 4.31 (m, 2H), 4.29 - 4.13 (m, 2H), 3.78 - 3.63 (m, 2H), |
| | 2.16 - 1.82 (m, 11H), 1.55 (d, J = 15.3, 1.3 Hz, 1H), 1.29 (s, |
| | 1H), 0.94 (s, 9H), 0.64 (s, 9H). |
| 1390 | ¹H NMR (400 MHZ, DMSO-d6) 8 13.07 (s, 1H), 8.69 (s, 1H), |
| | 8.53 (s, 2H), 7.95 (s, 1H), 7.66 (s, 2H), 7.25 (d, J = 8.0 Hz, 1H), |
| | 7.13 (s, 2H), 6.41 (s, 1H), 5.38 (d, J = 9.8 Hz, 1H), 4.86 (d, J = |
| | 16.5 Hz, 1H), 4.66 (d, J = 16.6 Hz, 1H), 4.18 (t, J = 11.0 Hz, |
| | 1H), 4.10 - 4.02 (m, 1H), 4.02 - 3.96 (m, 2H), 2.23 - 1.99 (m, |
| | 3H), 1.99 - 1.84 (m, 3H), 1.78 (dd, J = 15.1, 8.9 Hz, 1H), 1.40 |
| | (d, J = 15.0 Hz, 1H), 1.31 - 1.17 (m, 1H), 0.63 - 0.58 (m, 2H), |
| | 0.57 (s, 9H), 0.38 - 0.30 (m, 2H). |
| 1391 | ¹H NMR (400 MHZ, DMSO-d6) 8 13.06 (s, 1H), 8.63 (s, 1H), |
| | 8.53 (s, 1H), 7.93 (s, 1H), 7.66 (s, 2H), 7.26 (t, J = 7.5 Hz, 1H), |
| | 7.13 (d, J = 7.6 Hz, 2H), 6.39 (s, 1H), 5.36 (d, J = 9.8 Hz, 1H), |
| | 4.88 (d, J = 16.6 Hz, 1H), 4.71 - 4.54 (m, 2H), 4.21 (t, J = 11.1 |
| | Hz, 1H), 4.02 (s, 1H), 2.25 - 1.82 (m, 6H), 1.81 - 1.52 (m, 3H), |
| | 1.41 (s, 1H), 1.34 - 1.07 (m, 6H), 0.93 (t, J = 7.4 Hz, 3H), 0.75 |
| | (d, J = 6.6 Hz, 3H), 0.27 (d, J = 6.4 Hz, 2H). |
| 1392 | ¹H NMR (400 MHZ, Chloroform-d) 8 8.96 (s, 1H), 8.38 (s, 2H), |
| | 8.11 (d, J = 7.6 Hz, 1H), 7.85 (d, J = 7.1 Hz, 1H), 7.66 (t, J = |
| | 7.7 Hz, 1H), 7.22 (t, J = 7.6 Hz, 1H), 7.06 (d, J = 7.6 Hz, 2H), |
| | 6.24 (s, 1H), 5.58 (dd, J = 11.3, 4.1 Hz, 1H), 5.28 (d, J = 16.3 |
| | Hz, 1H), 4.97 (s, 1H), 4.37 (d, J = 16.4 Hz, 1H), 4.21 - 4.12 (m, |
| | 1H), 4.04 - 3.96 (m, 3H), 3.96 - 3.89 (m, 1H), 3.86 (t, J = 11.4 |
| | Hz, 1H), 2.34 - 2.23 (m, 1H), 2.19 - 2.11 (m, 1H), 2.06 - 2.00 |
| | (m, 6H), 1.78 - 1.63 (m, 1H), 1.52 (d, J = 14.8 Hz, 1H), 0.65 (s, |
| | 9H). Sulfonamide NH not observed - exchangeable proton in |
| | CDC13. |
| 1394 | ¹H NMR (400 MHZ, DMSO-d6) 8 13.30 - 12.60 (bs, 1H), 8.61 |
| | (s, 1H), 7.98 (s, 1H), 7.98 - 7.91 (m, 1H), 7.91 (s, 1H), 7.68 (s, |
| | 2H), 7.26 (t, J = 7.8 Hz, 1H), 7.13 (d, J = 7.2 Hz, 2H), 6.45 (s, |
| | 1H), 5.41 - 5.28 (m, 1H), 4.87 (p, J = 8.1 Hz, 1H), 4.71 (d, J = |
| | 15.5 Hz, 1H), 4.42 (d, J = 15.6 Hz, 1H), 4.35 (t, J = 10.5 Hz, |
| | 1H), 4.13 - 4.06 (m, 1H), 3.81 (td, J = 15.1, 9.4 Hz, 2H), 3.54 - |
| | 3.44 (m, 2H), 3.18 (s, 3H), 2.48 - 2.42 (m, 1H), 2.39 (dd, J = |
| | 10.7, 8.5 Hz, 1H), 2.35 - 2.17 (m, 2H), 2.16 - 1.85 (bs, 6H), 1.77 |
| | (dd, J = 15.2, 8.9 Hz, 1H), 1.42 (d, J = 14.9 Hz, 1H), 0.56 (s, |
| | 9H), 0.38 (s, 4H) |
| 1395 | ¹H NMR (400 MHZ, DMSO-d6) 8 13.07 (s, 1H), 8.63 (s, 1H), |
| | 8.53 (s, 2H), 7.93 (s, 1H), 7.74 - 7.56 (m, 2H), 7.25 (q, J = 10.5, |
| | 9.0 Hz, 1H), 7.13 (d, J = 7.6 Hz, 2H), 6.37 (d, J = 17.0 Hz, 1H), |
| | 5.43 - 5.29 (m, 1H), 4.88 (d, J = 16.6 Hz, 1H), 4.69 - 4.52 (m, |
| | 2H), 4.21 (t, J = 11.1 Hz, 1H), 4.09 - 3.93 (m, 1H), 2.19 - 1.87 |
| | (m, 6H), 1.81 - 1.57 (m, 3H), 1.47 - 1.35 (m, 1H), 1.32 - 1.12 |
| | (m, 5H), 0.93 (t, J = 7.4 Hz, 3H), 0.75 (d, J = 6.6 Hz, 3H), 0.27 |
| | (d, J = 6.4 Hz, 2H). |
| 1396 | ¹H NMR (400 MHZ, DMSO-d6) 8 13.06 (s, 1H), 8.70 (s, 1H), |
| | 8.54 (s, 2H), 7.94 (s, 1H), 7.66 (s, 2H), 7.34 - 7.22 (m, 1H), 7.12 |
| | (d, J = 7.6 Hz, 2H), 6.41 (s, 1H), 5.43 - 5.33 (m, 1H), 4.86 (d, |
| | J = 16.4 Hz, 1H), 4.66 (d, J = 16.5 Hz, 1H), 4.19 (t, J = 11.2 |
| | Hz, 1H), 4.08 - 3.99 (m, 1H), 3.93 (d, J = 6.5 Hz, 2H), 2.20 - |
| | 1.83 (m, 7H), 1.78 (dd, J = 15.2, 8.8 Hz, 1H), 1.40 (d, J = 14.9 |
| | Hz, 1H), 0.98 (d, J = 6.7 Hz, 6H), 0.57 (s, 9H). |
| 1397 | ¹H NMR (400 MHZ, DMSO-d6) 8 13.08 (s, 1H), 8.69 (s, 1H), |
| | 8.67 (d, J = 1.2 Hz, 1H), 8.53 (s, 1H), 7.95 (s, 1H), 7.67 (s, 2H), |
| | 7.33 - 7.22 (m, 1H), 7.13 (d, J = 7.6 Hz, 2H), 6.90 (dd, J = 12.9, |
| | 3.2 Hz, 1H), 6.52 - 6.40 (m, 1H), 5.44 - 5.32 (m, 1H), 5.04 - |
| | 4.89 (m, 1H), 4.90 - 4.77 (m, 1H), 4.76 - 4.63 (m, 1H), 4.23 (q, |
| | J = 12.1 Hz, 1H), 4.11 - 4.00 (m, 1H), 3.30 - 3.20 (m, 1H), 3.21 - |
| | 3.00 (m, 1H), 2.83 - 2.70 (m, 1H), 2.24 - 2.00 (m, 3H), 2.00 - |
| | 1.85 (m, 3H), 1.78 (dd, J = 15.2, 8.8 Hz, 1H), 1.40 (d, J = 15.1 |
| | Hz, 1H), 0.57 (s, 9H). |
| 1398 | ¹H NMR (400 MHZ, DMSO-d6) 8 13.06 (s, 1H), 8.71 (s, 1H), |
| | 7.94 (s, 1H), 7.67 (s, 2H), 7.48 (dd, J = 8.3, 7.4 Hz, 1H), 7.25 |
| | (d, J = 7.9 Hz, 1H), 7.13 (s, 2H), 6.72 (dd, J = 7.9, 5.1 Hz, 2H), |
| | 6.43 (s, 1H), 5.43 (dd, J = 10.8, 4.3 Hz, 1H), 4.68 (d, J = 15.7 |
| | Hz, 1H), 4.56 (s, 2H), 4.39 (d, J = 15.7 Hz, 1H), 4.27 (t, J = |
| | 11.2 Hz, 1H), 4.05 (d, J = 9.9 Hz, 1H), 2.07 (s, 7H), 1.75 (dd, |
| | J = 15.2, 8.9 Hz, 1H), 1.70 - 1.55 (m, 4H), 1.46 - 1.37 (m, 4H), |
| | 0.55 (s, 9H). |
| 1499 | ¹H NMR (400 MHz, DMSO-d6) 8 13.10 (s, 1H), 8.63 (s, 1H), |
| | 7.95 (t, J = 4.8 Hz, 1H), 7.69 (d, J = 4.7 Hz, 2H), 7.55 (t, J = |
| | 7.9 Hz, 1H), 7.26 (t, J = 7.6 Hz, 1H), 7.12 (d, J = 7.7 Hz, 2H), |
| | 6.70 (dd, J = 13.4, 7.9 Hz, 2H), 6.42 (s, 1H), 5.25 (dd, J = 10.9, |
| | 4.2 Hz, 1H), 4.79 (d, J = 15.8 Hz, 1H), 4.39 (d, J = 15.8 Hz, |
| | 1H), 4.29 (d, J = 5.9 Hz, 1H), 4.19 (t, J = 11.1 Hz, 1H), 4.09 - |
| | 3.92 (m, 4H), 3.65 (d, J = 8.9 Hz, 4H), 3.30 (q, J = 5.5, 3.7 Hz, |
| | 1H), 3.14 (s, 3H), 1.95 (d, J = 36.5 Hz, 6H), 1.77 (dd, J = 15.1, |
| | 8.9 Hz, 1H), 1.39 (d, J = 15.0 Hz, 1H), 0.54 (s, 9H). |
| 1403 | ¹H NMR (400 MHz, DMSO-d6) 8 13.01 (s, 1H), 8.69 (s, 1H), |
| | 8.03 (s, 2H), 7.90 (s, 1H), 7.61 (s, 2H), 7.21 (s, 1H), 7.09 (s, |
| | 2H), 6.37 (s, 1H), 5.36 (d, J = 8.8 Hz, 1H), 4.83 (d, J = 16.3 |
| | Hz, 1H), 4.53 (d, J = 16.5 Hz, 1H), 4.06 (t, J = 7.5 Hz, 3H), |
| | 3.49 (t, J = 6.5 Hz, 2H), 2.85 (h, J = 6.8 Hz, 1H), 1.97 (s, 6H), |
| | 1.74 (s, 2H), 1.39 (d, J = 14.9 Hz, 1H), 1.23 (d, J = 6.8 Hz, |
| | 3H), 0.56 (s, 9H). |
| 1404 | ¹H NMR (400 MHZ, DMSO-d6) 8 8.70 (t, J = 1.8 Hz, 1H), 8.06 |
| | (s, 2H), 7.82 (d, J = 7.6 Hz, 1H), 7.49 (d, J = 24.5 Hz, 2H), |
| | 7.13 (s, 1H), 7.04 (d, J = 7.5 Hz, 2H), 5.82 (s, 1H), 5.32 (d, J = |
| | 6.7 Hz, 1H), 4.82 (d, J = 16.2 Hz, 1H), 4.55 - 4.43 (m, 2H), |
| | 4.20 (dd, J = 8.0, 6.5 Hz, 2H), 3.80 (s, 1H), 3.69 - 3.59 (m, 3H), |
| | 1.93 (d, J = 21.4 Hz, 6H), 1.70 (t, J = 12.6 Hz, 1H), 1.38 (d, J = |
| | 14.8 Hz, 1H), 1.23 (s, 2H), 1.10 (d, J = 6.1 Hz, 6H), 0.53 (s, |
| | 9H). |
| 1405 | ¹H NMR (400 MHZ, DMSO-d6) 8 8.70 (t, J = 1.8 Hz, 1H), 8.06 |
| | (s, 2H), 7.79 (dt, J = 7.7, 1.4 Hz, 1H), 7.48 (t, J = 7.6 Hz, 1H), |
| | 7.41 (dt, J = 7.6, 1.5 Hz, 1H), 7.13 - 7.07 (m, 1H), 7.01 (d, J = |
| | 7.5 Hz, 2H), 5.75 (s, 1H), 5.31 (dd, J = 10.9, 4.6 Hz, 1H), 4.81 |
| | (d, J = 16.2 Hz, 1H), 4.45 (d, J = 16.3 Hz, 1H), 4.22 (qd, J = |
| | 13.2, 12.2, 5.4 Hz, 2H), 3.93 (ddd, J = 8.7, 7.3, 3.9 Hz, 1H), |
| | 3.74 (t, J = 11.2 Hz, 1H), 3.63 (q, J = 8.0 Hz, 1H), 2.40 (ddd, |
| | J = 12.0, 6.9, 3.6 Hz, 1H), 2.06 (dq, J = 10.7, 8.2 Hz, 2H), 1.95 |
| | (s, 6H), 1.67 (dd, J = 15.1, 9.3 Hz, 1H), 1.43 (d, J = 6.1 Hz, |
| | 3H), 1.41 - 1.34 (m, 1H), 0.52 (s, 9H). |
| 1406 | ¹H NMR (400 MHZ, DMSO-d6) 8 8.70 (d, J = 1.8 Hz, 1H), 8.06 |
| | (s, 2H), 7.82 (d, J = 7.7 Hz, 1H), 7.56 - 7.41 (m, 2H), 7.13 (t, J = |
| | 7.1 Hz, 1H), 7.03 (d, J = 7.5 Hz, 2H), 5.81 (s, 1H), 5.31 (dd, |
| | J = 10.8, 4.5 Hz, 1H), 4.82 (d, J = 16.3 Hz, 1H), 4.46 (d, J = |
| | 16.3 Hz, 1H), 4.21 (h, J = 6.4 Hz, 2H), 3.93 (ddd, J = 8.7, 7.3, |
| | 3.8 Hz, 1H), 3.78 (s, 1H), 3.62 (q, J = 8.0 Hz, 1H), 2.46 - 2.37 |
| | (m, 1H), 2.11 - 2.01 (m, 2H), 1.93 (d, J = 20.6 Hz, 5H), 1.69 |
| | (dd, J = 15.1, 9.3 Hz, 1H), 1.42 (d, J = 6.1 Hz, 3H), 1.38 (d, J = |
| | 14.9 Hz, 1H), 1.23 (s, 1H), 0.53 (s, 9H). |
| 1408 | ¹H NMR (400 MHZ, DMSO) 8 8.63 (s, 1H), 8.51 (s, 1H), 7.93 |
| | (d, J = 6.6 Hz, 1H), 7.66 (s, 1H), 7.26 (t, J = 7.6 Hz, 1H), 7.13 |
| | (d, J = 7.6 Hz, 1H), 6.39 (s, 1H), 5.36 (dd, J = 10.8, 4.2 Hz, |
| | 1H), 5.01 (s, 1H), 4.89 (d, J = 16.5 Hz, 1H), 4.68 - 4.58 (m, |
| | 1H), 4.48 (s, 4H), 4.20 (t, J = 11.1 Hz, 1H), 4.02 (s, 1H), 3.79 - |
| | 3.62 (m, 3H), 3.55 - 3.42 (m, 2H), 2.03 (s, 5H), 1.95 (s, 1H), |
| | 1.78 - 1.66 (m, 4H), 1.60 (s, 1H), 1.35 (d, J = 4.0 Hz, 1H), 1.24 |
| | (s, 3H), 1.21 (s, 1H), 0.75 (d, J = 6.6 Hz, 2H), 0.27 (d, J = 6.4 |
| | Hz, 2H). |
| 1409 | ¹H NMR (400 MHZ, DMSO-d6) 8 13.03 (br. s, 1H), 8.49 (br. s, |
| | 1H), 7.99 - 7.85 (m, 1H), 7.73 - 7.56 (m, 2H), 7.42 (s, 1H), 7.31 - |
| | 7.19 (m, 1H), 7.17 - 7.05 (m, 2H), 6.39 (br. s, 1H), 5.21 (dd, |
| | J = 10.5, 4.4 Hz, 1H), 4.53 (d, J = 15.4 Hz, 1H), 4.38 - 4.23 |
| | (m, 1H), 4.16 (d, J = 15.4 Hz, 1H), 3.97 - 3.81 (m, 1H), 3.59 - |
| | 3.46 (m, 4H), 3.24 (s, 3H), 3.02 (s, 3H), 2.29 - 1.80 (m, 7H), |
| | 1.37 (d, J = 14.7 Hz, 1H), 0.51 (s, 9H). |
| 1410 | ¹H NMR (400 MHZ, DMSO-d6) & 13.00 (br. s, 1H), 8.49 (br. s, |
| | 1H), 7.92 (br. s, 1H), 7.65 (br. s, 2H), 7.43 (s, 1H), 7.31 - 7.18 |
| | (m, 1H), 7.17 - 7.03 (m, 2H), 6.39 (br. s, 1H), 5.21 (dd, J = |
| | 11.4, 4.3 Hz, 1H), 4.54 (d, J = 15.4 Hz, 1H), 4.32 (t, J = 11.2 |
| | Hz, 1H), 4.16 (d, J = 15.2 Hz, 1H), 3.93 - 3.80 (m, 1H), 3.61 - |
| | 3.40 (m, 5H), 3.03 (s, 3H), 2.26 - 1.81 (m, 7H), 1.37 (d, J = |
| | 14.9 Hz, 1H), 1.03 (d, J = 2.7 Hz, 3H), 1.01 (d, J = 2.7 Hz, 3H), |
| | 0.51 (s, 9H). |
| 1411 | ¹H NMR (400 MHZ, DMSO-d6) 8 13.26 - 12.37 (br. s, 1H), 8.42 |
| | (s, 1H), 7.94 (d, J = 5.1 Hz, 1H), 7.75 - 7.62 (m, 2H), 7.26 (t, J = |
| | 7.8 Hz, 1H), 7.12 (d, J = 7.6 Hz, 2H), 6.70 (s, 1H), 6.43 (br. |
| | s, 1H), 5.10 (dd, J = 10.6, 4.3 Hz, 1H), 4.72 (d, J = 15.9 Hz, |
| | 1H), 4.46 - 4.31 (m, 2H), 3.96 - 3.86 (m, 1H), 3.59 - 3.45 (m, |
| | 4H), 3.23 (s, 3H), 3.02 (s, 3H), 2.21 - 1.88 (m, 6H), 1.83 (dd, J = |
| | 15.2, 9.0 Hz, 1H), 1.38 (d, J = 14.7 Hz, 1H), 0.50 (s, 9H). |
| 1303 | ¹H NMR (400 MHZ, DMSO-d6) 8 13.05 (br. s., 1H), 8.86 (br. |
| | s., 1H), 8.02 - 7.89 (m, 2H), 7.66 (br. s., 2H), 7.25 (m, 1H), 7.16 |
| | (s, 1H), 7.15 - 7.06 (m, 3H), 6.47 - 6.30 (m, 1H), 5.59 (d, J = |
| | 8.8 Hz, 1H), 5.03 (d, J = 16.1 Hz, 1H), 4.62 (d, J = 16.1 Hz, |
| | 1H), 4.23 - 4.10 (m, 1H), 4.08 - 3.99 (m, 1H), 3.80 (s, 3H), 2.23 - |
| | 1.88 (m, 7H), 1.82 (dd, J = 15.2, 8.3 Hz, 1H), 1.46 (d, J = |
| | 15.2 Hz, 1H), 0.89 - 0.81 (m, 2H), 0.60 (m, 11H). |
| 1412 | ¹H NMR (400 MHz, DMSO-d6) 8 13.13 (br. s., 1H), 8.65 (d, J = |
| | 2.9 Hz, 1H), 8.62 (br. s., 1H), 8.01 - 7.87 (m, 2H), 7.75 - 7.59 |
| | (m, 3H), 7.25 (d, J = 7.8 Hz, 1H), 7.13 (br. s., 2H), 6.42 (br. s., |
| | 1H), 5.32 (d, J = 6.8 Hz, 1H), 4.86 (d, J = 15.9 Hz, 1H), 4.65 |
| | (d, J = 15.9 Hz, 1H), 4.29 (t, J = 10.8 Hz, 1H), 4.05 (br. s., 1H), |
| | 2.22 - 2.03 (m, 3H), 2.01 - 1.87 (m, 3H), 1.78 (dd, J = 15.0, 8.9 |
| | Hz, 1H), 1.41 (d, J = 14.9 Hz, 1H), 0.54 (s, 9H). |
| 1413 | ¹H NMR (400 MHZ, DMSO-d6) & 13.18 (s, 1H), 8.49 (s, 1H), |
| | 7.93 (d, J = 6.4 Hz, 1H), 7.68 (d, J = 5.4 Hz, 2H), 7.26 (t, J = |
| | 7.6 Hz, 1H), 7.12 (d, J = 7.7 Hz, 2H), 6.39 (s, 1H), 6.03 (s, 1H), |
| | 5.09 (dd, J = 10.9, 4.3 Hz, 1H), 4.75 (d, J = 15.2 Hz, 1H), 4.31 |
| | (d, J = 15.3 Hz, 1H), 4.16 (t, J = 11.2 Hz, 1H), 3.93 - 3.85 (m, |
| | 1H), 3.74 (s, 3H), 3.00 (h, J = 6.8 Hz, 1H), 2.15 - 1.85 (m, 7H), |
| | 1.34 (d, J = 15.0 Hz, 1H), 1.19 (dd, J = 9.2, 6.8 Hz, 6H), 0.50 |
| | (s, 9H). |
| 1414 | ¹H NMR (400 MHZ, DMSO-d6) 8 13.11 (s, 1H), 8.67 (s, 1H), |
| | 7.96 (t, J = 4.7 Hz, 1H), 7.70 (d, J = 4.7 Hz, 2H), 7.50 (s, 1H), |
| | 7.26 (t, J = 7.6 Hz, 1H), 7.12 (d, J = 7.7 Hz, 2H), 6.63 (s, 2H), |
| | 6.44 (s, 1H), 5.32 (dd, J = 10.7, 4.3 Hz, 1H), 4.74 (d, J = 15.6 |
| | Hz, 1H), 4.42 (s, 1H), 4.24 (t, J = 11.3 Hz, 1H), 4.07 (d, J = 9.6 |
| | Hz, 1H), 3.91 - 3.83 (m, 4H), 3.68 - 3.64 (m, 2H), 3.30 (s, 2H), |
| | 1.95 (d, J = 36.5 Hz, 6H), 1.77 (dd, J = 15.3, 8.9 Hz, 1H), 1.39 |
| | (d, J = 15.0 Hz, 1H), 0.83 (d, J = 7.2 Hz, 6H), 0.53 (s, 9H). |
| 1416 | ¹H NMR (400 MHZ, DMSO-d6) 8 13.08 (s, 1H), 8.70 (s, 1H), |
| | 8.64 (s, 2H), 8.07 - 7.84 (m, 1H), 7.67 (s, 2H), 7.26 (t, J = 7.6 |
| | Hz, 1H), 7.13 (d, J = 7.6 Hz, 2H), 6.43 (s, 1H), 6.43 (tt, J = |
| | 54.1, 3.4 Hz, 1H), 5.39 (dd, J = 11.0, 4.2 Hz, 1H), 4.87 (d, J = |
| | 16.6 Hz, 1H), 4.70 (d, J = 16.7 Hz, 1H), 4.55 (td, J = 14.7, 3.4 |
| | Hz, 2H), 4.21 (t, J = 11.2 Hz, 1H), 4.12 - 3.95 (m, 1H), 1.94 (s, |
| | 6H), 1.78 (dd, J = 15.2, 8.8 Hz, 1H), 1.40 (d, J = 15.0 Hz, 1H), |
| | 0.57 (s, 9H). |
| 1419 | ¹H NMR (400 MHZ, DMSO) § 8.67 (s, 1H), 8.05 - 7.91 (m, |
| | 1H), 7.71 (s, 2H), 7.27 (t, J = 7.6 Hz, 1H), 7.13 (d, J = 7.7 Hz, |
| | 2H), 6.83 - 6.54 (m, 3H), 6.46 (s, 1H), 5.38 - 5.24 (m, 1H), 4.83 - |
| | 4.67 (m, 1H), 4.38 - 4.21 (m, 1H), 4.12 - 3.98 (m, 2H), 3.88 - |
| | 3.80 (m, 2H), 3.70 - 3.60 (m, 2H), 3.47 - 3.26 (m, 2H), 2.18 - |
| | 1.84 (m, 8H), 1.79 - 1.68 (m, 1H), 1.51 - 1.34 (m, 3H), 0.52 (s, |
| | 9H). |
| 1420 | ¹H NMR (400 MHZ, DMSO) & 8.63 (s, 1H), 7.96 (d, J = 6.8 |
| | Hz, 1H), 7.79 (s, 1H), 7.73 (s, 1H), 7.71 - 7.65 (m, 2H), 7.26 (t, |
| | J = 7.6 Hz, 1H), 7.19 - 7.04 (m, 2H), 6.45 (s, 1H), 5.35 (dd, J = |
| | 11.0, 4.3 Hz, 1H), 4.67 (d, J = 15.7 Hz, 1H), 4.38 (d, J = 15.7 |
| | Hz, 1H), 4.30 (t, J = 11.2 Hz, 1H), 4.08 - 3.96 (m, 2H), 3.82 - |
| | 3.69 (m, 2H), 3.56 - 3.47 (m, 2H), 3.44 - 3.30 (m, 1H), 2.27 - |
| | 1.81 (m, 8H), 1.74 (dd, J = 15.2, 9.0 Hz, 1H), 1.47 - 1.34 (m, |
| | 3H), 0.56 (s, 9H). |
| 1305 | ¹H NMR (400 MHZ, DMSO) 8 13.03 (s, 1H), 8.65 (s, 1H), 8.59 |
| | (d, J = 2.4 Hz, 1H), 8.14 (d, J = 2.4 Hz, 1H), 7.94 (d, J = 5.7 |
| | Hz, 1H), 7.68 (d, J = 4.8 Hz, 2H), 7.26 (t, J = 7.6 Hz, 1H), 7.13 |
| | (d, J = 7.6 Hz, 2H), 6.39 (s, 1H), 5.29 (dd, J = 10.8, 4.3 Hz, |
| | 1H), 5.14 - 4.97 (m, 2H), 4.52 (d, J = 18.0 Hz, 1H), 4.23 (q, J = |
| | 11.1 Hz, 1H), 4.03 (tt, J = 11.2, 3.9 Hz, 1H), 3.78 - 3.59 (m, |
| | 3H), 3.64 - 3.40 (m, 2H), 1.95 (s, 6H), 1.95 (h, J = 6.8 Hz, 1H), |
| | 1.78 - 1.67 (m, 4H), 1.71 - 1.58 (m, 1H), 1.58 (dt, J = 10.9, 5.3 |
| | Hz, 1H), 1.51 - 1.34 (m, 1H), 1.28 - 1.15 (m, 2H), 0.76 (d, J = |
| | 6.6 Hz, 3H), 0.27 (d, J = 6.4 Hz, 3H). |
| 1422 | ¹H NMR (400 MHZ, DMSO-d6) 8 13.07 (s, 1H), 8.69 (s, 1H), |
| | 8.44 (s, 2H), 7.95 (s, 1H), 7.67 (s, 2H), 7.25 (d, J = 7.7 Hz, 1H), |
| | 7.17 - 7.06 (m, 2H), 6.42 (s, 1H), 5.41 - 5.33 (m, 1H), 4.94 (p, |
| | J = 6.9 Hz, 1H), 4.84 (d, J = 16.5 Hz, 1H), 4.66 (d, J = 16.5 |
| | Hz, 1H), 4.20 (t, J = 11.1 Hz, 1H), 4.09 - 4.00 (m, 1H), 2.42 - |
| | 2.31 (m, 2H), 2.24 - 2.01 (m, 3H), 2.00 - 1.89 (m, 3H), 1.86 (dd, |
| | J = 11.2, 6.5 Hz, 2H), 1.77 (dd, J = 15.1, 8.8 Hz, 1H), 1.39 (d, |
| | J = 14.9 Hz, 1H), 1.16 (d, J = 7.3 Hz, 6H), 0.56 (s, 9H). |
| 1424 | ¹H NMR (400 MHZ, DMSO-d6) & 12.92 (br. s., 1H), 8.42 (s, |
| | 1H), 8.00 - 7.90 (m, 1H), 7.68 (br. s., 2H), 7.26 (t, J = 7.6 Hz, |
| | 1H), 7.17 - 7.09 (m, 2H), 6.77 (s, 1H), 6.43 (br. s., 1H), 5.11 |
| | (dd, J = 10.5, 4.2 Hz, 1H), 4.73 (d, J = 15.9 Hz, 1H), 4.44 (d, J = |
| | 15.7 Hz, 1H), 4.36 (t, J = 11.1 Hz, 1H), 3.94 - 3.86 (m, 1H), |
| | 3.71 - 3.64 (m, 4H), 3.41 - 3.35 (m, 4H), 2.19 - 1.87 (m, 6H), |
| | 1.82 (dd, J = 15.2, 9.0 Hz, 1H), 1.39 (d, J = 14.9 Hz, 1H), |
| | 0.50(s, 9H). |
| 1425 | ¹H NMR (400 MHZ, DMSO-d6) 8 13.10 (br. s., 1H), 9.04 (s, |
| | 2H), 8.70 (br. s., 1H), 7.96 (d, J = 6.1 Hz, 1H), 7.67 (br. s., 2H), |
| | 7.32 - 7.21 (m, 1H), 7.18 - 7.07 (m, 2H), 6.43 (br. s., 1H), 5.44 - |
| | 5.33 (m, 1H), 4.97 - 4.79 (m, 2H), 4.30 (t, J = 11.2 Hz, 1H), |
| | 4.13 - 4.01 (m, 1H), 2.21 - 1.84 (m, 6H), 1.77 (dd, J = 15.2, 8.8 |
| | Hz, 1H), 1.41 (d, J = 14.7 Hz, 1H), 0.57 (s, 9H). |
| 1297 | ¹H NMR (400 MHZ, Chloroform-d) 8 9.97 (s, 1H), 8.87 (d, J = |
| | 1.8 Hz, 1H), 8.39 - 8.32 (m, 2H), 8.10 (d, J = 7.9 Hz, 1H), 7.81 |
| | (dt, J = 7.6, 1.4 Hz, 1H), 7.64 (t, J = 7.8 Hz, 1H), 7.19 (t, J = |
| | 7.6 Hz, 1H), 7.02 (d, J = 7.6 Hz, 2H), 6.16 (s, 1H), 5.49 (dd, J = |
| | 11.2, 4.1 Hz, 1H), 5.36 (d, J = 16.6 Hz, 1H), 4.60 (hept, J = |
| | 6.0 Hz, 1H), 4.26 (d, J = 16.6 Hz, 1H), 3.98 (tt, J = 11.1, 3.9 |
| | Hz, 1H), 3.78 (t, J = 11.4 Hz, 1H), 2.17 (hept, J = 7.8 Hz, 1H), |
| | 1.98 (s, 6H), 1.87 - 1.74 (m, 2H), 1.73 - 1.53 (m, 5H), 1.40 - |
| | 1.33 (m, 6H), 1.20 - 1.06 (m, 1H). ¹H NMR (400 MHZ, DMSO) |
| | 8 12.84 (s, 1H), 8.59 (s, 1H), 8.52 (s, 2H), 7.83 (s, 1H), 7.56 (s, |
| | 2H), 7.24 - 7.13 (m, 1H), 7.12 - 7.03 (m, 2H), 6.11 (s, 1H), 5.43 - |
| | 5.21 (m, 1H), 4.89 (d, J = 16.5 Hz, 1H), 4.80 (p, J = 6.0 Hz, |
| | 1H), 4.53 (d, J = 16.5 Hz, 1H), 4.01 - 3.92 (m, 2H), 2.13 - 1.93 |
| | (m, 7H), 1.89 - 1.76 (m, 2H), 1.67 (dt, J = 10.3, 8.5 Hz, 1H), |
| | 1.61 - 1.38 (m, 4H), 1.30 (dd, J = 6.0, 2.7 Hz, 6H), 0.84 (q, J = |
| | 8.2, 7.2 Hz, 1H). |
| 1426 | ¹H NMR (400 MHZ, DMSO-d6) § 13.36 - 12.66 (bs, 1H), 8.58 |
| | (s, 1H), 8.45 (s, 2H), 7.90 (s, 1H), 7.66 (s, 2H), 7.26 (t, J = 7.7 |
| | Hz, 1H), 7.13 (d, J = 7.4 Hz, 2H), 6.40 (s, 1H), 5.40 - 5.25 (m, |
| | 1H), 4.93 - 4.80 (m, 2H), 4.58 (d, J = 16.5 Hz, 1H), 4.19 (t, J = |
| | 11.2 Hz, 1H), 3.94 - 3.81 (m, 1H), 2.48 - 2.40 (m, 2H), 2.19 - |
| | 1.92 (m, 9H), 1.90 - 1.74 (m, 3H), 1.74 - 1.38 (m, 6H), 0.96 - |
| | 0.77 (m, 1H) |
| 1428 | ¹H NMR (400 MHZ, DMSO-d6) 8 13.00 (s, 1H), 8.69 (s, 1H), |
| | 7.92 (s, 1H), 7.65 (s, 2H), 7.49 - 7.42 (m, 1H), 7.23 (s, 1H), 7.10 |
| | (d, J = 7.7 Hz, 2H), 6.57 (d, J = 7.2 Hz, 1H), 6.35 (s, 1H), 6.24 |
| | (d, J = 8.4 Hz, 1H), 5.37 (d, J = 9.1 Hz, 1H), 4.73 (d, J = 15.7 |
| | Hz, 1H), 4.32 (d, J = 15.7 Hz, 1H), 4.14 (s, 1H), 4.06 (s, 1H), |
| | 3.95 (s, 1H), 3.58 (dd, J = 10.4, 6.5 Hz, 1H), 3.46 (d, J = 8.1 |
| | Hz, 1H), 2.20 - 1.86 (m, 9H), 1.78 (dt, J = 23.8, 7.8 Hz, 2H), |
| | 1.70 - 1.59 (m, 1H), 1.52 - 1.35 (m, 2H), 1.27 - 1.20 (m, 1H), |
| | 0.99 (d, J = 6.4 Hz, 3H), 0.90 (d, J = 6.6 Hz, 3H), 0.55 (s, 9H). |
| 1429 | ¹H NMR (400 MHZ, DMSO-d6) 8 12.99 (s, 1H), 8.69 (s, 1H), |
| | 7.92 (s, 1H), 7.65 (s, 2H), 7.45 (dd, J = 8.4, 7.2 Hz, 1H), 7.23 |
| | (s, 1H), 7.10 (d, J = 7.4 Hz, 2H), 6.57 (d, J = 7.3 Hz, 1H), 6.33 |
| | (s, 1H), 6.24 (d, J = 8.4 Hz, 1H), 5.37 (d, J = 9.4 Hz, 1H), 4.73 |
| | (d, J = 15.7 Hz, 1H), 4.32 (d, J = 15.8 Hz, 1H), 4.14 (s, 1H), |
| | 4.06 (s, 1H), 3.95 (s, 1H), 3.65 - 3.54 (m, 1H), 3.53 - 3.40 (m, |
| | 1H), 2.23 - 1.84 (m, 9H), 1.84 - 1.70 (m, 2H), 1.64 (ddt, J = |
| | 10.8, 6.8, 3.5 Hz, 1H), 1.52 - 1.36 (m, 2H), 1.29 - 1.21 (m, 1H), |
| | 0.99 (d, J = 6.5 Hz, 3H), 0.90 (d, J = 6.6 Hz, 3H), 0.54 (s, 9H). |
| 1430 | ¹H NMR (400 MHZ, DMSO-d6) 8 13.08 (s, 1H), 8.80 (s, 2H), |
| | 8.70 (s, 1H), 7.95 (s, 1H), 7.67 (s, 2H), 7.40 (t, J = 54 Hz, 1H), |
| | 7.26 (s, 1H), 7.12 (d, J = 7.6 Hz, 2H), 6.43 (s, 1H), 5.39 (d, J = |
| | 10.2 Hz, 1H), 4.90 (d, J = 16.8 Hz, 1H), 4.78 (d, J = 16.8 Hz, |
| | 1H), 4.24 (t, J = 12.6 Hz, 1H), 4.06 (s, 1H), 1.94 (s, 6H), 1.78 |
| | (dd, J = 15.3, 8.8 Hz, 1H), 1.41 (d, J = 15.0 Hz, 1H), 0.57 (s, |
| | 9H). |
| 1431 | ¹H NMR (400 MHZ, DMSO-d6) 8 13.08 (s, 1H), 8.70 (s, 1H), |
| | 8.69 (s, 2H), 7.96 (s, 1H), 7.67 (s, 2H), 7.26 (t, J = 7.5 Hz, 1H), |
| | 7.13 (d, J = 7.6 Hz, 2H), 6.43 (s, 1H), 5.40 (dd, J = 11.1, 4.1 |
| | Hz, 1H), 5.00 (q, J = 8.8 Hz, 2H), 4.88 (d, J = 16.6 Hz, 1H), |
| | 4.72 (d, J = 16.7 Hz, 1H), 4.22 (t, J = 11.2 Hz, 1H), 4.12 - 3.96 |
| | (m, 1H), 1.94 (s, 6H), 1.78 (dd, J = 15.2, 8.9 Hz, 1H), 1.41 (d, |
| | J = 15.0 Hz, 1H), 0.57 (s, 9H). |
| 1432 | ¹H NMR (400 MHZ, DMSO) § 8.68 - 8.61 (m, 1H), 8.47 (s, |
| | 2H), 7.78 (dt, J = 7.6, 1.5 Hz, 1H), 7.52 - 7.37 (m, 2H), 7.16 - |
| | 7.06 (m, 1H), 7.02 (d, J = 7.6 Hz, 2H), 5.75 (s, 1H), 5.31 (dd, |
| | J = 10.8, 4.4 Hz, 1H), 4.87 (d, J = 16.4 Hz, 1H), 4.63 - 4.48 |
| | (m, 2H), 4.22 (s, 1H), 4.09 (s, OH), 3.84 (t, J = 11.1 Hz, 1H), |
| | 3.77 - 3.54 (m, 2H), 3.17 (s, 1H), 3.15 (s, 2H), 3.00 - 2.84 (m, |
| | 2H), 1.97 (s, 5H), 2.02 - 1.83 (m, 2H), 1.74 - 1.45 (m, 1H), 1.42 - |
| | 1.30 (m, 1H), 1.24 (s, 2H), 1.30 - 1.12 (m, 1H), 0.92 - 0.78 |
| | (m, OH), 0.84 (s, 1H), 0.73 (d, J = 6.6 Hz, 3H), 0.24 (d, J = 6.4 |
| | Hz, 3H). |
| 1433 | ¹H NMR (400 MHZ, DMSO) 8 12.01 (s, 1H), 8.64 (d, J = 1.8 |
| | Hz, 1H), 8.58 (s, 1H), 7.82 (d, J = 7.5 Hz, 1H), 7.55 - 7.45 (m, |
| | 1H), 7.13 (d, J = 7.8 Hz, 1H), 7.04 (d, J = 7.5 Hz, 1H), 5.86 (s, |
| | 1H), 5.32 (dd, J = 10.9, 4.4 Hz, 1H), 4.88 (dd, J = 16.5, 1.4 Hz, |
| | 1H), 4.64 - 4.50 (m, 1H), 4.17 (s, 1H), 3.80 (dtd, J = 11.8, 3.2, |
| | 1.2 Hz, 1H), 3.58 (ttd, J = 11.0, 7.1, 3.3 Hz, 2H), 2.18 (t, J = |
| | 7.4 Hz, 1H), 1.98 (s, 5H), 1.73 (dd, J = 20.3, 8.8 Hz, 1H), 1.57 - |
| | 1.43 (m, 1H), 1.40 - 1.18 (m, 1H), 1.24 (s, 12H), 0.92 - 0.78 |
| | (m, 2H), 0.73 (d, J = 6.6 Hz, 2H), 0.25 (d, J = 6.4 Hz, 2H). |
| 1434 | ¹H NMR (400 MHZ, DMSO) 8 12.91 (s, 1H), 8.64 (t, J = 1.6 |
| | Hz, 1H), 8.54 (s, 2H), 7.98 - 7.90 (m, 1H), 7.73 - 7.64 (m, 2H), |
| | 7.26 (t, J = 7.6 Hz, 1H), 7.13 (d, J = 7.7 Hz, 2H), 6.39 (s, 1H), |
| | 5.37 (dd, J = 10.7, 4.3 Hz, 1H), 4.89 (d, J = 16.5 Hz, 1H), 4.63 |
| | (d, J = 16.8 Hz, 1H), 4.47 (p, J = 6.0 Hz, 1H), 4.21 (t, J = 11.2 |
| | Hz, 1H), 4.02 (td, J = 12.3, 10.2, 3.6 Hz, 1H), 3.78 - 3.63 (m, |
| | 2H), 3.63 - 3.42 (m, 2H), 2.03 (s, 6H), 1.90 (pd, J = 6.8, 5.5 Hz, |
| | 1H), 1.75 (ddd, J = 14.1, 10.3, 3.2 Hz, 1H), 1.41 (ddt, J = 9.9, |
| | 6.5, 3.2 Hz, OH), 1.22 (s, 1H), 1.28 - 1.15 (m, 3H), 0.95 (dd, J = |
| | 13.7, 6.8 Hz, 5H), 0.75 (d, J = 6.6 Hz, 3H), 0.28 (d, J = 6.4 |
| | Hz, 3H). |
| 1436 | ¹H NMR (400 MHZ, Methanol-d4) 8 8.97 (d, J = 1.9 Hz, 1H), |
| | 8.52 (d, J = 1.9 Hz, 1H), 8.19 - 7.87 (m, 2H), 7.83 - 7.60 (m, |
| | 2H), 7.28 (t, J = 7.6 Hz, 1H), 7.15 (d, J = 7.7 Hz, 2H), 6.31 (s, |
| | 1H), 5.73 - 5.53 (m, 1H), 5.17 - 4.94 (m, 1H), 4.77 (dd, J = |
| | 10.1, 2.8 Hz, 2H), 4.62 (d, J = 16.2 Hz, 1H), 4.33 - 4.09 (m, |
| | 2H), 4.06 - 3.84 (m, 3H), 3.84 - 3.59 (m, 2H), 3.43 (ddd, J = |
| | 11.4, 10.1, 1.1 Hz, 1H), 2.63 (s, 3H), 2.46 - 1.71 (m, 7H), 1.59 |
| | (d, J = 15.3 Hz, 1H), 0.66 (s, 9H). |
| 1438 | ¹H NMR (400 MHZ, DMSO-d6) 8 8.69 (s, 1H), 8.15 (s, 2H), |
| | 7.95 (d, J = 7.2 Hz, 1H), 7.74 - 7.60 (m, 2H), 7.26 (t, J = 7.6 |
| | Hz, 1H), 7.13 (d, J = 7.6 Hz, 2H), 6.42 (s, 1H), 5.38 (dd, J= |
| | 10.5, 4.1 Hz, 1H), 4.85 (d, J = 16.2 Hz, 1H), 4.57 (d, J = 16.3 |
| | Hz, 1H), 4.25 - 4.07 (m, 3H), 3.56 - 3.42 (m, 3H), 3.40 - 3.26 |
| | (m, 3H), 2.18 - 1.87 (m, 8H), 1.80 (dd, J = 15.2, 8.7 Hz, 2H), |
| | 1.43 - 1.30 (m, 1H), 1.10 (t, J = 7.0 Hz, 3H), 0.57 (s, 9H). |
| 1439 | ¹H NMR (400 MHZ, DMSO-d6) 8 8.68 (s, 1H), 8.13 (s, 2H), |
| | 7.95 (d, J = 7.3 Hz, 1H), 7.75 - 7.61 (m, 2H), 7.26 (t, J = 7.6 |
| | Hz, 1H), 7.13 (d, J = 7.6 Hz, 2H), 6.41 (s, 1H), 5.38 (dd, J = |
| | 10.5, 4.1 Hz, 1H), 4.84 (d, J = 16.2 Hz, 1H), 4.56 (d, J = 16.3 |
| | Hz, 1H), 4.11 (t, J = 11.1 Hz, 1H), 4.08 - 3.94 (m, 2H), 3.48 - |
| | 3.29 (m, 5H), 3.06 (dd, J = 9.7, 6.5 Hz, 1H), 2.58 (p, J = 7.1 |
| | Hz, 1H), 2.13 - 1.88 (m, 7H), 1.84 - 1.68 (m, 3H), 1.46 - 1.32 |
| | (m, 2H), 0.57 (s, 9H). |
| 1440 | ¹H NMR (400 MHZ, DMSO-d6) 8 13.05 (s, 1H), 8.69 (s, 1H), |
| | 8.13 (s, 2H), 7.94 (d, J = 7.2 Hz, 1H), 7.66 (s, 2H), 7.26 (t, J = |
| | 7.6 Hz, 1H), 7.12 (d, J = 7.6 Hz, 2H), 6.41 (s, 1H), 5.43 - 5.31 |
| | (m, 1H), 4.85 (d, J = 16.3 Hz, 1H), 4.55 (d, J = 16.3 Hz, 1H), |
| | 4.11 (t, J = 11.0 Hz, 1H), 4.03 (d, J = 8.1 Hz, 1H), 3.63 - 3.40 |
| | (m, 4H), 2.30 - 1.87 (m, 8H), 1.79 (dd, J = 15.2, 8.7 Hz, 1H), |
| | 1.58 (d, J = 20.9 Hz, 3H), 1.39 (d, J = 14.8 Hz, 1H), 0.57 (s, |
| | 9H). |
| 1441 | ¹H NMR (400 MHZ, DMSO-d6) 8 13.08 (s, 1H), 8.69 (s, 1H), |
| | 8.14 (s, 2H), 7.94 (d, J = 7.3 Hz, 1H), 7.67 (d, J = 9.0 Hz, 2H), |
| | 7.26 (t, J = 7.6 Hz, 1H), 7.12 (d, J = 7.7 Hz, 2H), 6.41 (s, 1H), |
| | 5.38 (dd, J = 10.6, 4.1 Hz, 1H), 4.84 (d, J = 16.3 Hz, 1H), 4.56 |
| | (d, J = 16.3 Hz, 1H), 4.12 (t, J = 11.1 Hz, 1H), 4.07 - 3.97 (m, |
| | 1H), 3.64 - 3.43 (m, 4H), 2.31 - 1.85 (m, 8H), 1.79 (dd, J = |
| | 15.2, 8.8 Hz, 1H), 1.58 (d, J = 20.9 Hz, 3H), 1.39 (d, J = 14.7 |
| | Hz, 1H), 0.57 (s, 9H). |
| 1442 | ¹H NMR (400 MHZ, DMSO-d6) 8 13.05 (s, 1H), 8.69 (s, 1H), |
| | 8.52 (s, 2H), 7.95 (s, 1H), 7.66 (s, 2H), 7.26 (t, J = 7.7 Hz, 1H), |
| | 7.12 (d, J = 7.7 Hz, 2H), 6.41 (s, 1H), 5.38 (s, 1H), 4.83 (d, J = |
| | 16.4 Hz, 1H), 4.59 (d, J = 16.5 Hz, 1H), 4.15 (t, J = 11.1 Hz, |
| | 1H), 4.04 (d, J = 9.7 Hz, 1H), 3.61 (dd, J = 10.5, 6.4 Hz, 2H), |
| | 3.12 - 3.01 (m, 2H), 2.00 (d, J = 52.7 Hz, 6H), 1.85 - 1.65 (m, |
| | 5H), 1.46 - 1.28 (m, 4H), 0.56 (s, 9H). |
| 1443 | ¹H NMR (400 MHZ, DMSO-d6) 8 13.06 (s, 1H), 8.70 (s, 1H), |
| | 8.58 (s, 2H), 7.95 (s, 1H), 7.67 (s, 2H), 7.27 (t, J = 7.6 Hz, 1H), |
| | 7.13 (d, J = 7.6 Hz, 2H), 6.42 (s, 1H), 5.48 - 5.31 (m, 1H), 4.84 |
| | (d, J = 16.4 Hz, 1H), 4.61 (d, J = 16.5 Hz, 1H), 4.17 (t, J = 11.1 |
| | Hz, 1H), 4.10 - 3.96 (m, 1H), 3.55 - 3.42 (m, 4H), 2.07 (tt, J = |
| | 14.0, 5.8 Hz, 7H), 1.95 (s, 3H), 1.79 (dd, J = 15.2, 8.8 Hz, 1H), |
| | 1.40 (d, J = 14.8 Hz, 1H), 0.57 (s, 9H). |
| 1444 | ¹H NMR (400 MHZ, DMSO-d6) 8 8.71 (s, 1H), 8.21 (s, 2H), |
| | 7.85 (s, 1H), 7.54 (s, 2H), 7.05 (s, 2H), 5.35 (d, J = 9.4 Hz, 1H), |
| | 4.84 (d, J = 16.4 Hz, 1H), 4.55 (d, J = 16.6 Hz, 1H), 4.42 (t, J = |
| | 12.3 Hz, 4H), 4.16 (s, 1H), 1.96 (s, 6H), 1.72 (s, 1H), 1.39 (d, |
| | J = 14.8 Hz, 1H), 1.23 (s, 2H), 0.54 (s, 9H). |
| 1445 | ¹H NMR (500 MHZ, dmso) 8 8.65 (s, 1H), 7.95 (s, 1H), 7.68 (s, |
| | 2H), 7.47 (s, 1H), 7.25 (t, J = 7.5 Hz, 1H), 7.11 (d, J = 7.3 Hz, |
| | 2H), 6.67 - 6.66 (m, 2H), 6.42 (s, 1H), 5.29 (dd, J = 10.7, 4.2 |
| | Hz, 1H), 4.88 (s, 1H), 4.70 (d, J = 15.8 Hz, 1H), 4.39 (d, J = |
| | 14.9 Hz, 1H), 4.25 - 4.16 (m, 1H), 4.11 - 4.02 (m, 1H), 4.01 - |
| | 3.93 (m, 1H), 3.89 - 3.88 (m, 1H), 3.81 - 3.75 (m, 1H), 3.63 - |
| | 3.56 (m, 1H), 3.45 (d, J = 15.0 Hz, 1H), 3.33 - 3.24 (m, 1H), |
| | 2.10 - 1.89 (m, 6H), 1.88 - 1.81 (m, 2H), 1.80 - 1.71 (m, 2H), |
| | 1.54 - 1.45 (m, 1H), 1.37 (d, J = 14.9 Hz, 1H), 1.21 (d, J = 6.6 |
| | Hz, 3H), 1.12 (d, J = 6.6 Hz, 3H), 0.52 (s, 9H). |
| 1446 | ¹H NMR (500 MHz, dmso) 8 8.64 (s, 1H), 7.95 (s, 1H), 7.68 (s, |
| | 2H), 7.51 (s, 1H), 7.25 (t, J = 7.6 Hz, 1H), 7.11 (d, J = 7.1 Hz, |
| | 2H), 6.62 (s, 2H), 6.43 (s, 1H), 5.29 (dd, J = 10.8, 4.3 Hz, 1H), |
| | 4.84 (s, 1H), 4.68 (d, J = 15.6 Hz, 1H), 4.39 (d, J = 14.4 Hz, |
| | 1H), 4.30 - 4.20 (m, 1H), 4.12 - 4.03 (m, 1H), 4.03 - 3.95 (m, |
| | 1H), 3.82 - 3.75 (m, 1H), 3.66 - 3.55 (m, 1H), 3.47 (d, J = 15.0 |
| | Hz, 1H), 3.34 - 3.24 (m, 1H), 2.14 - 1.89 (m, 6H), 1.89 - 1.81 |
| | (m, 2H), 1.80 - 1.70 (m, 2H), 1.79 - 1.40 (m, 1H), 1.37 (d, J = |
| | 14.9 Hz, 1H), 1.20 (d, J = 6.7 Hz, 3H), 1.11 (d, J = 6.5 Hz, 3H), |
| | 0.52 (s, 9H). |
| 1447 | ¹H NMR (400 MHz, DMSO-d6) 8 13.02 (s, 1H), 8.61 (s, 1H), |
| | 8.54 (s, 2H), 7.92 (s, 1H), 7.66 (s, 2H), 7.28 - 7.21 (m, 1H), 7.12 |
| | (d, J = 7.6 Hz, 2H), 6.32 (s, 1H), 5.36 (d, J = 10.3 Hz, 1H), |
| | 4.96 (d, J = 16.5 Hz, 1H), 4.63 - 4.56 (m, 2H), 4.19 (t, J = 11.2 |
| | Hz, 1H), 4.09 - 3.99 (m, 1H), 2.15 - 1.97 (m, 6H), 1.91 (s, 1H), |
| | 1.71 - 1.59 (m, 2H), 1.27 (d, J = 6.0 Hz, 3H), 1.18 (d, J = 6.3 |
| | Hz, 1H), 0.93 (t, J = 7.4 Hz, 3H), 0.46 (d, J = 6.4 Hz, 1H), 0.37 |
| | (dt, J = 13.3, 6.4 Hz, 1H), 0.21 - 0.12 (m, 1H), -0.02 (d, J = 4.5 |
| | Hz, 1H), -0.55 (s, 1H). |
| 1448 | ¹H NMR (400 MHz, DMSO) & 13.04 (s, 1H), 8.75 (s, 1H), 8.31 |
| | (dd, J = 5.1, 1.4 Hz, 1H), 7.97 (d, J = 7.0 Hz, 1H), 7.70 (d, J = |
| | 7.0 Hz, 2H), 7.26 (t, J = 7.6 Hz, 1H), 7.13 (d, J = 7.7 Hz, 2H), |
| | 6.72 (d, J = 5.1 Hz, 1H), 6.45 (s, 1H), 5.52 (q, J = 8.3 Hz, 1H), |
| | 5.43 (dd, J = 10.8, 4.3 Hz, 1H), 4.68 (d, J = 16.5 Hz, 1H), 4.48 |
| | (d, J = 16.6 Hz, 1H), 4.28 (t, J = 11.1 Hz, 1H), 4.12 - 4.01 (m, |
| | 1H), 3.20 (s, 3H), 2.46 (t, J = 9.5 Hz, 2H), 2.16 (q, J = 9.9 Hz, |
| | 3H), 2.02 (s, 6H), 1.73 (dd, J = 15.3, 8.9 Hz, 1H), 1.41 (d, J = |
| | 15.0 Hz, 1H), 0.56 (s, 9H), 0.41 (s, 3H). |
| 1449 | ¹H NMR (400 MHZ, DMSO) 8 12.94 (s, 1H), 8.77 (s, 1H), 8.29 |
| | (dd, J = 5.6, 2.4 Hz, 1H), 8.01 - 7.91 (m, 1H), 7.70 (q, J = 7.7 |
| | Hz, 2H), 7.26 (t, J = 7.6 Hz, 1H), 7.13 (d, J = 7.7 Hz, 2H), 6.78 |
| | (s, 1H), 6.44 (s, 1H), 5.48 (d, J = 9.4 Hz, 1H), 4.65 (dd, J = |
| | 61.5, 12.7 Hz, 2H), 4.28 (s, 2H), 4.09 - 3.98 (m, 1H), 3.38 (q, J = |
| | 7.0 Hz, 3H), 2.14 - 1.92 (m, 8H), 1.75 - 1.65 (m, 2H), 1.42 |
| | (d, J = 15.0 Hz, 1H), 1.32 - 1.19 (m, 2H), 1.09 (t, J = 7.0 Hz, |
| | 1H), 0.56 (s, 9H). |
| 1453 | ¹H NMR (400 MHZ, DMSO-d6) 8 13.01 (s, 1H), 8.61 (s, 1H), |
| | 8.54 (s, 2H), 7.92 (s, 1H), 7.66 (s, 2H), 7.30 - 7.20 (m, 1H), 7.12 |
| | (d, J = 7.6 Hz, 2H), 6.32 (s, 1H), 5.35 (d, J = 10.0 Hz, 1H), |
| | 4.97 (d, J = 16.5 Hz, 1H), 4.64 - 4.60 (m, 1H), 4.60 - 4.52 (m, |
| | 1H), 4.18 (t, J = 11.2 Hz, 1H), 4.09 - 3.98 (m, 1H), 2.19 - 1.95 |
| | (m, 6H), 1.93 - 1.85 (m, 1H), 1.73 - 1.57 (m, 2H), 1.26 (d, J = |
| | 6.0 Hz, 3H), 1.19 - 1.09 (m, 1H), 0.93 (t, J = 7.4 Hz, 3H), 0.51 - |
| | 0.41 (m, 1H), 0.40 - 0.31 (m, 1H), 0.21 - 0.11 (m, 1H), -0.00 - |
| | - 0.09 (m, 1H), -0.55 (s, 1H). |
| 1454 | ¹H NMR (400 MHZ, DMSO-d6) 8 13.01 (s, 1H), 8.60 (s, 1H), |
| | 8.45 (s, 2H), 7.92 (s, 1H), 7.66 (s, 2H), 7.26 (t, J = 7.7 Hz, 1H), |
| | 7.12 (d, J = 7.6 Hz, 2H), 6.32 (s, 1H), 5.35 (d, J = 10.1 Hz, |
| | 1H), 5.00 - 4.88 (m, 2H), 4.59 (d, J = 16.5 Hz, 1H), 4.18 (t, J = |
| | 11.2 Hz, 1H), 4.08 - 3.98 (m, 1H), 2.42 - 2.31 (m, 2H), 2.19 - |
| | 1.91 (m, 7H), 1.91 - 1.78 (m, 3H), 1.16 (d, J = 7.2 Hz, 6H), |
| | 0.54 - 0.44 (m, 1H), 0.42 - 0.31 (m, 1H), 0.22 - 0.10 (m, 1H), - |
| | 0.00 - - 0.07 (m, 1H), -0.56 (s, 1H). |
| 1455 | ¹H NMR (400 MHZ, DMSO-d6) 8 13.02 (s, 1H), 8.61 (s, 1H), |
| | 8.54 (s, 2H), 7.92 (s, 1H), 7.66 (s, 2H), 7.31 - 7.21 (m, 1H), 7.12 |
| | (d, J = 7.7 Hz, 2H), 6.32 (s, 1H), 5.35 (d, J = 10.3 Hz, 1H), |
| | 4.96 (d, J = 16.6 Hz, 1H), 4.59 (d, J = 16.6 Hz, 1H), 4.47 (p, J = |
| | 6.1 Hz, 1H), 4.18 (t, J = 11.2 Hz, 1H), 4.09 - 3.97 (m, 1H), |
| | 2.19 - 1.94 (m, 6H), 1.93 - 1.85 (m, 2H), 1.21 (d, J = 6.1 Hz, |
| | 3H), 1.18 - 1.11 (m, 1H), 0.95 (dd, J = 13.5, 6.8 Hz, 6H), 0.51 - |
| | 0.42 (m, 1H), 0.41 - 0.32 (m, 1H), 0.21 - 0.13 (m, 1H), -0.00 - |
| | - 0.06 (m, 1H), -0.55 (s, 1H). |
| 1456 | ¹H NMR (400 MHZ, DMSO-d6) 8 13.05 (br. s., 1H), 8.53 (br. |
| | s., 1H), 7.98 - 7.86 (m, 1H), 7.72 - 7.59 (m, 3H), 7.30 - 7.22 (m, |
| | 1H), 7.11 (d, J = 6.1 Hz, 2H), 6.38 (br. s., 1H), 6.19 (d, J = 2.2 |
| | Hz, 1H), 5.22 (dd, J = 10.6, 4.3 Hz, 1H), 4.78 (d, J = 15.2 Hz, |
| | 1H), 4.40 (d, J = 15.4 Hz, 1H), 4.18 (t, J = 11.0 Hz, 1H), 4.07 - |
| | 3.98 (m, 1H), 3.90 - 3.78 (m, 2H), 3.62 (dd, J = 10.8, 2.9 Hz, |
| | 1H), 3.25 (t, J = 11.0 Hz, 1H), 2.98 (t, J = 11.2 Hz, 1H), 2.21 - |
| | 1.83 (m, 8H), 1.62 (d, J = 11.7 Hz, 1H), 1.43 (d, J = 6.6 Hz, |
| | 3H), 1.37 (d, J = 14.7 Hz, 1H), 1.20 (qd, J = 12.5, 4.8 Hz, 1H), |
| | 1.05 (qd, J = 12.4, 4.6 Hz, 1H), 0.82 (d, J = 12.7 Hz, 1H), 0.51 |
| | (s, 9H). |
| 1457 | ¹H NMR (400 MHZ, DMSO-d6) 8 13.06 (br. s., 1H), 8.51 (br. |
| | s., 1H), 8.02 - 7.85 (m, 1H), 7.76 - 7.59 (m, 3H), 7.25 (t, J = |
| | 7.8 Hz, 1H), 7.11 (d, J = 7.6 Hz, 2H), 6.38 (br. s., 1H), 6.20 (d, |
| | J = 2.2 Hz, 1H), 5.12 (dd, J = 10.6, 4.0 Hz, 1H), 4.84 (d, J = |
| | 15.2 Hz, 1H), 4.39 (d, J = 15.4 Hz, 1H), 4.17 - 4.01 (m, 2H), |
| | 3.89 -3.82 (m, 2H), 3.73 (dd, J = 11.0, 2.9 Hz, 1H), 3.26 (t, J = |
| | 10.9 Hz, 1H), 3.11 (t, J = 10.9 Hz, 1H), 2.19 - 1.84 (m, 8H), |
| | 1.62 (d, J = 13.0 Hz, 1H), 1.41 (d, J = 6.8 Hz, 3H), 1.35 (d, J = |
| | 15.2 Hz, 1H), 1.29 - 1.08 (m, 2H), 0.92 (d, J = 11.7 Hz, 1H), |
| | 0.51 (s, 9H). |
| 1458 | ¹H NMR (400 MHZ, DMSO-d6) 8 13.10 (br. s, 1H), 8.63 (br. s., |
| | 1H), 7.95 (br. s., 1H), 7.70 (br. s., 2H), 7.47 (t, J = 7.9 Hz, 1H), |
| | 7.31 - 7.21 (m, 1H), 7.18 - 7.07 (m, 2H), 6.61 (d, J = 7.3 Hz, |
| | 1H), 6.39 (d, J = 8.6 Hz, 2H), 5.28 (t, J = 5.1 Hz, 1H), 5.18 (d, |
| | J = 5.1 Hz, 1H), 4.78 (d, J = 15.7 Hz, 1H), 4.32 (d, J = 15.2 |
| | Hz, 1H), 4.04 (d, J = 5.6 Hz, 2H), 3.85 (dd, J = 10.8, 6.1 Hz, |
| | 1H), 3.63 - 3.55 (m, 1H), 3.51 - 3.44 (m, 1H), 3.41 - 3.35 (m, |
| | 1H), 2.36 - 2.25 (m, 1H), 2.17 - 1.81 (m, 11H), 1.74 (dd, J = |
| | 14.1, 7.5 Hz, 1H), 1.35 (d, J = 15.2 Hz, 1H), 0.70 (t, J = 7.3 |
| | Hz, 3H), 0.52 (s, 9H). |
| 1459 | ¹H NMR (400 MHZ, DMSO-d6) § 13.05 (br. s, 1H), 8.63 (br. s., |
| | 1H), 7.95 (br. s., 1H), 7.70 (br. s., 2H), 7.47 (t, J = 7.9 Hz, 1H), |
| | 7.31 - 7.21 (m, 1H), 7.16 - 7.06 (m, 2H), 6.61 (d, J = 7.3 Hz, |
| | 1H), 6.38 (d, J = 8.6 Hz, 2H), 5.32 - 5.24 (m, 1H), 5.18 (d, J = |
| | 5.4 Hz, 1H), 4.78 (d, J = 15.7 Hz, 1H), 4.32 (d, J = 15.9 Hz, |
| | 1H), 4.10 - 3.97 (m, 2H), 3.85 (dd, J = 10.6, 6.2 Hz, 1H), 3.64 - |
| | 3.54 (m, 1H), 3.50 - 3.42 (m, 1H), 3.41 - 3.35 (m, 1H), 2.34 - |
| | 2.25 (m, 1H), 2.18 - 1.81 (m, 11H), 1.77 - 1.70 (m, 1H), 1.35 |
| | (d, J = 15.2 Hz, 1H), 0.70 (t, J = 7.3 Hz, 3H), 0.52 (s, 9H). |
| 1460 | ¹H NMR (400 MHZ, DMSO-d6) 8 13.26 - 12.13 (br. s, 1H), 8.42 |
| | (s, 1H), 7.93 (d, J = 5.6 Hz, 1H), 7.74 - 7.61 (m, 2H), 7.25 (t, J = |
| | 7.8 Hz, 1H), 7.12 (d, J = 7.3 Hz, 2H), 6.70 (s, 1H), 6.41 (br. |
| | s., 1H), 5.11 (dd, J = 10.5, 4.2 Hz, 1H), 4.72 (d, J = 15.7 Hz, |
| | 1H), 4.45 - 4.29 (m, 2H), 3.98 - 3.86 (m, 1H), 3.59 - 3.40 (m, |
| | 5H), 3.03 (s, 3H), 2.07 (br. s, 6H), 1.83 (dd, J = 15.2, 9.0 Hz, |
| | 1H), 1.39 (d, J = 14.7 Hz, 1H), 1.01 (d, J = 6.1 Hz, 6H), 0.50 |
| | (s, 9H). |
| 1304 | ¹H NMR (400 MHZ, DMSO-d6) 8 13.06 (br. s., 1H), 8.89 (br. |
| | s., 1H), 7.94 (br. s., 1H), 7.78 (d, J = 7.8 Hz, 1H), 7.67 (br. s., |
| | 2H), 7.35 - 7.20 (m, 1H), 7.18 - 7.04 (m, 3H), 6.40 (br. s., 1H), |
| | 6.08 (s, 1H), 5.61 (d, J = 8.1 Hz, 1H), 5.01 (d, J = 15.7 Hz, |
| | 1H), 4.61 (d, J = 15.7 Hz, 1H), 4.29 - 4.12 (m, 1H), 4.09 - 4.00 |
| | (m, 1H), 3.91 (s, 3H), 2.22 - 1.89 (m, 7H), 1.82 (dd, J = 14.8, |
| | 8.9 Hz, 1H), 1.46 (d, J = 14.9 Hz, 1H), 1.05 - 0.92 (m, 2H), |
| | 0.76 - 0.66 (m, 2H), 0.60 (s, 9H). |
| 1461 | ¹H NMR (400 MHZ, DMSO) 8 8.63 (d, J = 2.0 Hz, 1H), 8.46 |
| | (s, 2H), 7.94 (dt, J = 6.6, 2.2 Hz, 1H), 7.73 - 7.62 (m, 2H), 7.26 |
| | (t, J = 7.6 Hz, 1H), 7.13 (d, J = 7.6 Hz, 2H), 6.39 (s, 1H), 5.36 |
| | (dd, J = 10.8, 4.3 Hz, 1H), 4.99 - 4.83 (m, 1H), 4.69 - 4.50 (m, |
| | 2H), 4.21 (dd, J = 13.0, 9.3 Hz, 1H), 4.02 (ddt, J = 11.2, 7.3, |
| | 3.6 Hz, 1H), 3.69 (h, J = 6.9 Hz, 1H), 3.36 (q, J = 7.0 Hz, 2H), |
| | 2.99 - 2.84 (m, 2H), 2.03 (s, 6H), 1.94 (dt, J = 12.0, 7.2 Hz, |
| | 2H), 1.75 (ddd, J = 14.1, 10.4, 3.2 Hz, 1H), 1.40 (dtt, J = 12.6, |
| | 9.5, 4.8 Hz, 1H), 1.34 - 1.16 (m, 4H), 1.10 (t, J = 7.0 Hz, 3H), |
| | 0.75 (d, J = 6.6 Hz, 3H), 0.27 (d, J = 6.4 Hz, 3H). |
| 1462 | ¹H NMR (400 MHZ, DMSO) 8 13.01 (s, 1H), 8.63 (s, 1H), 8.44 |
| | (s, 2H), 7.93 (d, J = 5.8 Hz, 1H), 7.70 - 7.63 (m, 2H), 7.26 (t, J = |
| | 7.6 Hz, 1H), 7.13 (d, J = 7.6 Hz, 2H), 6.39 (s, 1H), 5.36 (dd, |
| | J = 10.8, 4.2 Hz, 1H), 5.02 (tt, J = 6.9, 4.2 Hz, 1H), 4.95 - 4.84 |
| | (m, 1H), 4.63 (d, J = 16.7 Hz, 1H), 4.20 (t, J = 11.1 Hz, 1H), |
| | 4.13 - 3.97 (m, 1H), 3.17 (s, 2H), 2.46 (dtd, J = 11.1, 5.5, 4.1, |
| | 2.4 Hz, 2H), 2.33 (ddt, J = 10.8, 7.5, 4.2 Hz, 2H), 2.02 (s, 7H), |
| | 1.75 (ddd, J = 14.1, 10.3, 3.1 Hz, 1H), 1.47 - 1.34 (m, OH), 1.38 |
| | (s, 1H), 1.34 - 1.08 (m, 3H), 0.75 (d, J = 6.6 Hz, 3H), 0.27 (d, |
| | J = 6.4 Hz, 3H). |
| 1463 | ¹H NMR (400 MHZ, DMSO) & 12.99 (s, 1H), 8.61 (s, 1H), 8.51 |
| | (s, 2H), 7.92 (s, 1H), 7.66 (s, 2H), 7.25 (t, J = 7.6 Hz, 1H), 7.12 |
| | (d, J = 7.6 Hz, 2H), 6.31 (s, 1H), 5.35 (dd, J = 10.8, 3.9 Hz, |
| | 1H), 5.02 (td, J = 5.8, 4.8, 2.9 Hz, 1H), 4.97 (d, J = 16.6 Hz, |
| | 1H), 4.59 (d, J = 16.5 Hz, 1H), 4.17 (t, J = 11.2 Hz, 1H), 4.05 |
| | (s, 1H), 4.11 - 3.98 (m, OH), 2.12 - 1.82 (m, 10H), 1.80 - 1.66 |
| | (m, 4H), 1.60 (qd, J = 5.7, 2.7 Hz, 2H), 1.19 - 1.08 (m, 1H), |
| | 0.53 - 0.31 (m, 2H), 0.17 (dd, J = 7.9, 3.9 Hz, 1H), -0.56 (s, |
| | 1H). |
| 1464 | ¹H NMR (400 MHZ, DMSO-d6) 8 12.95 (s, 1H), 8.60 (s, 1H), |
| | 8.09 (s, 2H), 7.91 (s, 1H), 7.66 (s, 2H), 7.25 (t, J = 7.6 Hz, 1H), |
| | 7.12 (d, J = 7.6 Hz, 2H), 6.31 (s, 1H), 5.33 (dd, J = 10.4, 3.6 |
| | Hz, 1H), 4.96 (d, J = 16.2 Hz, 1H), 4.46 (d, J = 16.2 Hz, 1H), |
| | 4.13 - 3.96 (m, 2H), 3.39 (t, J = 7.0 Hz, 2H), 3.08 (s, 2H), 2.05 |
| | (d, J = 16.5 Hz, 6H), 1.93 - 1.85 (m, 1H), 1.77 (t, J = 7.0 Hz, |
| | 2H), 1.21 - 1.13 (m, 1H), 1.10 (s, 6H), 0.46 (dd, J = 7.9, 5.4 Hz, |
| | 1H), 0.41 - 0.33 (m, 1H), 0.17 (tt, J = 9.5, 4.4 Hz, 1H), -0.02 |
| | (dd, J = 7.8, 3.3 Hz, 1H), -0.55 (s, 1H). |
| 1465 | ¹H NMR (400 MHZ, DMSO) 8 13.12 (s, 1H), 8.70 (s, 1H), 8.31 |
| | (d, J = 5.0 Hz, 1H), 7.96 (d, J = 6.7 Hz, 1H), 7.70 (d, J = 6.6 |
| | Hz, 2H), 7.26 (t, J = 7.6 Hz, 1H), 7.13 (d, J = 7.6 Hz, 2H), 6.68 |
| | (d, J = 5.1 Hz, 1H), 6.45 (s, 1H), 5.38 (d, J = 10.8 Hz, 1H), |
| | 4.83 (tt, J = 11.8, 4.1 Hz, 1H), 4.65 (d, J = 16.3 Hz, 1H), 4.44 |
| | (d, J = 16.4 Hz, 1H), 4.29 (t, J = 11.1 Hz, 1H), 4.08 (d, J = 9.5 |
| | Hz, 1H), 3.91 (d, J = 10.9 Hz, 2H), 3.36 (d, J = 8.0 Hz, 2H), |
| | 3.05 (s, 3H), 1.99 (s, 7H), 1.87 - 1.67 (m, 2H), 1.54 (d, J = 12.2 |
| | Hz, 2H), 1.40 (d, J = 14.8 Hz, 1H), 0.55 (s, 9H). |
| 1467 | ¹H NMR (400 MHZ, DMSO-d6) 8 13.02 (s, 1H), 8.50 (s, 1H), |
| | 8.14 (d, J = 2.4 Hz, 1H), 7.91 (s, 1H), 7.66 (s, 2H), 7.45 (dd, J = |
| | 12.1, 2.4 Hz, 1H), 7.26 (t, J = 7.7 Hz, 1H), 7.12 (d, J = 7.7 |
| | Hz, 2H), 6.32 (s, 1H), 5.44 - 5.25 (m, 1H), 5.03 (dd, J = 15.7, |
| | 1.5 Hz, 1H), 4.74 (hept, J = 6.0 Hz, 1H), 4.38 (d, J = 15.7 Hz, |
| | 1H), 4.21 (t, J = 11.2 Hz, 1H), 4.01 (t, J = 11.0 Hz, 1H), 1.97 |
| | (d, J = 39.8 Hz, 7H), 1.29 (d, J = 6.0 Hz, 6H), 1.13 (d, J = 10.5 |
| | Hz, 1H), 0.52 - 0.43 (m, 1H), 0.38 (tq, J = 8.9, 4.2 Hz, 1H), |
| | 0.18 (s, 1H), -0.01 (m, 1H), -0.54 (s, 1H). |
| 1468 | ¹H NMR (400 MHZ, DMSO-d6) 8 13.02 (s, 1H), 8.50 (s, 1H), |
| | 8.18 - 8.05 (m, 1H), 7.90 (s, 1H), 7.66 (s, 2H), 7.32 (dd, J = |
| | 11.8, 2.4 Hz, 1H), 7.25 (d, J = 7.5 Hz, 1H), 7.12 (d, J = 7.7 Hz, |
| | 2H), 6.32 (s, 1H), 5.31 (d, J = 10.1 Hz, 1H), 5.02 (d, J = 15.4 |
| | Hz, 1H), 4.82 (p, J = 7.1 Hz, 1H), 4.38 (d, J = 15.7 Hz, 1H), |
| | 4.22 (t, J = 11.2 Hz, 1H), 4.03 (d, J = 10.8 Hz, 1H), 2.47 (s, |
| | 2H), 2.30 - 1.85 (m, 9H), 1.86 - 1.73 (m, 1H), 1.63 (qt, J = 10.2, |
| | 8.1 Hz, 1H), 1.16 (d, J = 9.7 Hz, 1H), 0.46 (t, J = 6.7 Hz, 1H), |
| | 0.42 - 0.33 (m, J = 3.9 Hz, 1H), 0.18 (d, J = 4.2 Hz, 1H), -0.00 |
| | (s, 1H), -0.54 (s, 1H). |
| 1469 | ¹H NMR (400 MHZ, DMSO-d6) 8 13.01 (s, 1H), 8.60 (s, 1H), |
| | 8.24 (s, 2H), 7.90 (s, 1H), 7.65 (s, 2H), 7.24 (s, 1H), 7.11 (d, J = |
| | 7.5 Hz, 2H), 6.30 (s, 1H), 5.34 (d, J = 9.6 Hz, 1H), 4.97 (d, J = |
| | 16.3 Hz, 1H), 4.51 (d, J = 16.4 Hz, 1H), 4.05 (s, 2H), 3.80 (t, |
| | J = 13.2 Hz, 2H), 3.58 (t, J = 7.2 Hz, 2H), 2.57 (d, J = 7.2 Hz, |
| | 2H), 2.01 (s, 6H), 1.90 (d, J = 4.9 Hz, 2H), 1.12 (d, J = 15.8 |
| | Hz, 1H), 0.45 (s, 1H), 0.41 - 0.33 (m, 1H), 0.17 (s, 1H), -0.56 |
| | (s, 1H). |
| 1471 | ¹H NMR (400 MHZ, DMSO) 8 8.65 (s, 1H), 8.51 (s, 2H), 7.91 |
| | (s, 1H), 7.65 (s, 2H), 7.33 - 7.22 (m, 1H), 7.13 (d, J = 7.6 Hz, |
| | 2H), 6.37 (s, 1H), 5.43 (dd, J = 11.7, 3.9 Hz, 1H), 4.96 (d, J = |
| | 16.5 Hz, 1H), 4.81 (p, J = 6.0 Hz, 1H), 4.60 - 4.44 (m, 2H), |
| | 3.92 (ddd, J = 11.8, 7.9, 4.1 Hz, 1H), 2.19 - 1.93 (m, 6H), 1.37 - |
| | 1.26 (m, 7H), 1.25 - 1.12 (m, 2H), 0.85 (d, J = 6.9 Hz, 3H), |
| | 0.36 (t, J = 7.3 Hz, 3H). |
| 1472 | ¹H NMR (400 MHZ, DMSO) 8 8.64 (s, 1H), 8.44 (s, 2H), 7.91 |
| | (s, 1H), 7.65 (s, 2H), 7.31 - 7.22 (m, 1H), 7.13 (d, J = 7.7 Hz, |
| | 2H), 6.36 (s, 1H), 5.43 (d, J = 11.2 Hz, 1H), 4.99 - 4.82 (m, |
| | 2H), 4.60 - 4.44 (m, 2H), 3.92 (ddd, J = 11.7, 7.9, 4.1 Hz, 1H), |
| | 2.49 - 2.40 (m, 2H), 2.17 - 1.91 (m, 8H), 1.86 - 1.74 (m, 1H), |
| | 1.72 - 1.55 (m, 1H), 1.38 - 1.11 (m, 3H), 0.85 (d, J = 6.9 Hz, |
| | 3H), 0.35 (t, J = 7.3 Hz, 3H). |
| 1473 | ¹H NMR (400 MHZ, DMSO) § 13.33 - 11.69 (m, 1H), 8.64 (s, |
| | 1H), 8.51 (s, 2H), 7.90 (s, 1H), 7.65 (s, 2H), 7.25 (d, J = 7.9 |
| | Hz, 1H), 7.13 (d, J = 7.5 Hz, 2H), 6.36 (s, 1H), 5.43 (d, J = |
| | 11.1 Hz, 1H), 4.96 (d, J = 16.5 Hz, 1H), 4.81 (hept, J = 6.0 Hz, |
| | 1H), 4.55 (d, J = 16.7 Hz, 1H), 4.48 (d, J = 11.8 Hz, 1H), 2.00 |
| | (s, 9H), 1.30 (dd, J = 6.0, 1.9 Hz, 7H), 0.85 (d, J = 6.9 Hz, 3H), |
| | 0.35 (t, J = 7.3 Hz, 3H). |
| 1474 | ¹H NMR (400 MHZ, DMSO) & 13.46 - 11.49 (m, 1H), 8.64 (s, |
| | 1H), 8.44 (s, 2H), 7.90 (s, 1H), 7.64 (s, 2H), 7.25 (s, 1H), 7.13 |
| | (s, 2H), 6.36 (s, 1H), 5.42 (d, J = 11.1 Hz, 1H), 4.95 (d, J = |
| | 16.5 Hz, 1H), 4.91 - 4.84 (m, 1H), 4.55 (d, J = 16.6 Hz, 1H), |
| | 4.52 - 4.43 (m, 1H), 3.93 (s, 1H), 2.48 - 2.42 (m, 2H), 2.14 - |
| | 2.02 (m, 5H), 2.00 (s, 4H), 1.81 (q, J = 10.3 Hz, 1H), 1.64 (dtd, |
| | J = 18.4, 10.2, 8.2 Hz, 1H), 1.35 - 1.27 (m, 1H), 1.18 (dd, J = |
| | 14.6, 7.5 Hz, 1H), 0.84 (d, J = 6.9 Hz, 3H), 0.35 (t, J = 7.3 Hz, |
| | 3H). |
| 1475 | ¹H NMR (400 MHz, DMSO) 8 13.51 - 11.50 (m, 1H), 8.77 (s, |
| | 1H), 8.26 (d, J = 5.0 Hz, 1H), 7.95 (s, 1H), 7.68 (s, 2H), 7.25 |
| | (d, J = 8.1 Hz, 1H), 7.12 (d, J = 7.7 Hz, 2H), 6.64 (d, J = 5.0 |
| | Hz, 1H), 6.42 (s, 1H), 5.45 (s, 1H), 4.65 (d, J = 16.3 Hz, 1H), |
| | 4.42 (d, J = 16.5 Hz, 1H), 4.24 (s, 1H), 4.06 (s, 2H), 3.68 (s, |
| | 2H), 2.41 (dq, J = 13.5, 6.8 Hz, 1H), 2.18 - 1.92 (m, 6H), 1.84 |
| | (s, 3H), 1.71 (dd, J = 15.3, 9.0 Hz, 1H), 1.41 (d, J = 15.0 Hz, |
| | 1H), 1.24 (s, 1H), 0.83 (s, 3H), 0.75 (d, J = 6.8 Hz, 3H), 0.56 |
| | (s, 9H). |
| 1476 | ¹H NMR (400 MHZ, DMSO) 8 13.33 - 11.76 (m, 1H), 8.72 (s, |
| | 1H), 8.26 (d, J = 5.0 Hz, 1H), 7.95 (s, 1H), 7.67 (s, 2H), 7.26 |
| | (t, J = 7.7 Hz, 1H), 7.12 (d, J = 7.6 Hz, 2H), 6.64 (d, J = 5.0 |
| | Hz, 1H), 6.43 (s, 1H), 5.48 (d, J = 8.9 Hz, 1H), 4.59 (s, 1H), |
| | 4.43 (d, J = 16.1 Hz, 1H), 4.26 (s, 2H), 4.02 (d, J = 8.8 Hz, |
| | 1H), 3.72 (s, 1H), 3.56 (s, 1H), 1.93 (s, 6H), 1.87 (s, 4H), 1.70 |
| | (dd, J = 15.2, 9.0 Hz, 1H), 1.41 (d, J = 15.0 Hz, 1H), 1.24 (s, |
| | 1H), 0.86 (d, J = 6.9 Hz, 3H), 0.73 (d, J = 6.9 Hz, 3H), 0.56 (s, |
| | 9H). |
| 1477 | ¹H NMR (400 MHZ, DMSO-d6) 8 13.57 - 11.57 (bs, 1H), 8.58 |
| | (s, 1H), 8.09 (s, 2H), 7.95 - 7.82 (m, 1H), 7.74 - 7.56 (m, 2H), |
| | 7.26 (t, J = 7.6 Hz, 1H), 7.13 (d, J = 7.6 Hz, 2H), 6.40 (s, 1H), |
| | 5.32 (dd, J = 10.8, 4.2 Hz, 1H), 4.90 (d, J = 16.1 Hz, 1H), 4.45 |
| | (d, J = 16.2 Hz, 1H), 4.09 (t, J = 11.2 Hz, 1H), 3.84 (tt, J = |
| | 10.9, 3.8 Hz, 1H), 3.39 (t, J = 7.0 Hz, 2H), 3.08 (s, 2H), 2.17 - |
| | 2.05 (m, 1H), 2.05 (s, 6H), 1.92 - 1.81 (m, 2H), 1.77 (t, J = 7.0 |
| | Hz, 2H), 1.73 - 1.63 (m, 1H), 1.62 - 1.40 (m, 4H), 1.10 (s, 6H), |
| | 0.89 (p, J = 9.8, 9.0 Hz, 1H) |
| 1478 | ¹H NMR (400 MHZ, DMSO-d6) 8 13.03 (s, 1H), 8.60 (s, 1H), |
| | 8.47 (s, 2H), 7.92 (s, 1H), 7.66 (s, 2H), 7.30 - 7.22 (m, 1H), 7.16 - |
| | 7.06 (m, 2H), 6.32 (s, 1H), 5.39 - 5.29 (m, 1H), 5.17 - 5.06 |
| | (m, 1H), 4.96 (d, J = 16.5 Hz, 1H), 4.60 (d, J = 16.6 Hz, 1H), |
| | 4.18 (t, J = 11.2 Hz, 1H), 4.09 - 3.99 (m, 1H), 2.57 - 2.52 (m, |
| | 1H), 2.48 - 2.43 (m, 1H), 2.40 - 2.29 (m, 2H), 2.21 (s, 1H), 2.16 - |
| | 1.90 (m, 6H), 1.90 - 1.80 (m, 1H), 1.20 - 1.07 (m, 1H), 0.55 - |
| | 0.48 (m, 2H), 0.48 - 0.43 (m, 2H), 0.41 - 0.33 (m, 1H), 0.22 - |
| | 0.11 (m, 1H), -0.00 - - 0.07 (m, 1H), -0.56 (s, 1H). |
| 1479 | ¹H NMR (400 MHZ, DMSO-d6) 8 13.84 - 12.16 (bs, 1H), 8.58 |
| | (s, 1H), 8.23 (s, 2H), 7.95 - 7.83 (m, 1H), 7.67 (s, 2H), 7.26 (t, |
| | J = 7.6 Hz, 1H), 7.13 (d, J = 7.6 Hz, 2H), 6.41 (s, 1H), 5.33 |
| | (dd, J = 10.8, 4.3 Hz, 1H), 4.90 (d, J = 16.4 Hz, 1H), 4.50 (d, J = |
| | 16.4 Hz, 1H), 4.12 (t, J = 11.2 Hz, 1H), 3.92 - 3.80 (m, 1H), |
| | 3.80 (t, J = 13.3 Hz, 2H), 3.58 (t, J = 7.2 Hz, 2H), 2.62 - 2.51 |
| | (m, 2H), 2.17 - 2.04 (m, 1H), 2.04 (s, 6H), 1.92 - 1.79 (m, 2H), |
| | 1.76 - 1.62 (m, 1H), 1.62 - 1.53 (m, 2H), 1.53 - 1.40 (m, 2H), |
| | 0.88 (p, J = 8.8 Hz, 1H) |
| 1481 | ¹H NMR (400 MHZ, DMSO-d6) 8 13.05 (s, 1H), 8.69 (s, 1H), |
| | 8.20 (s, 2H), 7.95 (d, J = 7.4 Hz, 1H), 7.67 (d, J = 9.8 Hz, 2H), |
| | 7.26 (t, J = 7.6 Hz, 1H), 7.13 (d, J = 7.7 Hz, 2H), 6.42 (s, 1H), |
| | 5.50 (dt, J = 13.0, 3.4 Hz, 1H), 5.43 - 5.32 (m, 2H), 4.84 (d, J = |
| | 16.3 Hz, 1H), 4.58 (d, J = 16.4 Hz, 1H), 4.13 (t, J = 11.1 Hz, |
| | 1H), 4.03 (ddd, J = 11.6, 8.5, 3.5 Hz, 1H), 3.75 (ddd, J = 20.5, |
| | 11.0, 5.2 Hz, 2H), 3.57 (ddt, J = 18.9, 11.2, 3.6 Hz, 2H), 2.24 - |
| | 1.84 (m, 6H), 1.79 (dd, J = 15.2, 8.8 Hz, 1H), 1.39 (d, J = 14.9 |
| | Hz, 1H), 0.57 (s, 9H). |
| 1482 | ¹H NMR (400 MHZ, Methanol-d4) 8 8.81 (t, J = 1.7 Hz, 1H), |
| | 8.50 (s, 2H), 8.01 (dt, J = 7.1, 1.8 Hz, 1H), 7.82 - 7.49 (m, 2H), |
| | 7.26 (t, J = 7.6 Hz, 1H), 7.13 (d, J = 7.6 Hz, 2H), 6.19 (s, 2H), |
| | 5.60 (dd, J = 10.3, 3.4 Hz, 1H), 5.16 (d, J = 16.6 Hz, 1H), 4.54 |
| | (d, J = 16.6 Hz, 1H), 4.20 (q, J = 6.9 Hz, 2H), 4.17 - 4.04 (m, |
| | 2H), 2.11 (s, 6H), 1.91 (ddd, J = 15.7, 10.2, 5.7 Hz, 1H), 1.44 |
| | (t, J = 7.0 Hz, 3H), 1.30 (ddd, J = 15.1, 7.7, 3.4 Hz, 2H), 0.62 - |
| | 0.37 (m, 2H), 0.28 (tt, J = 9.0, 5.0 Hz, 1H), 0.09 (dq, J = 9.6, |
| | 4.7 Hz, 1H). |
| 1485 | ¹H NMR (400 MHZ, DMSO-d6) 8 12.83 (br. s., 1H), 8.42 (s, |
| | 1H), 7.94 (d, J = 6.6 Hz, 1H), 7.74 - 7.62 (m, 2H), 7.26 (t, J = |
| | 7.6 Hz, 1H), 7.12 (d, J = 7.3 Hz, 2H), 6.72 (s, 1H), 6.42 (br. s., |
| | 1H), 5.10 (dd, J = 10.5, 4.2 Hz, 1H), 4.70 (d, J = 15.9 Hz, 1H), |
| | 4.44 (d, J = 15.9 Hz, 1H), 4.36 (t, J = 11.0 Hz, 1H), 3.90 (m, |
| | 1H), 3.39 (br. s., 4H), 2.07 (br. s, 6H), 1.83 (dd, J = 15.2, 9.0 |
| | Hz, 1H), 1.56 (br. s., 6H), 1.39 (d, J = 14.7 Hz, 1H), 0.50 (s, |
| | 9H). |
| 1296 | ¹H NMR (400 MHZ, Chloroform-d) 8 10.08 (broad s, 1H), 8.87 |
| | (s, 1H), 8.37 (s, 2H), 8.21 (d, J = 7.9 Hz, 1H), 7.82 (d, J = 7.6 |
| | Hz, 1H), 7.69 (t, J = 7.8 Hz, 1H), 7.18 (t, J = 7.6 Hz, 1H), 7.00 |
| | (d, J = 7.6 Hz, 2H), 6.14 (s, 1H), 5.51 (dd, J = 11.3, 4.0 Hz, |
| | 1H), 5.37 (d, J = 16.5 Hz, 1H), 4.60 (hept, J = 6.1 Hz, 1H), |
| | 4.25 (d, J = 16.6 Hz, 1H), 3.99 (s, 1H), 3.81 (t, J = 11.4 Hz, |
| | 1H), 1.96 (s, 6H), 1.63 - 1.56 (overlapped with water m, 2H), |
| | 1.42 - 1.34 (m, 6H), 1.25 - 1.16 (m, 1H), 0.90 - 0.79 (m, 10H). |
| 1486 | ¹H NMR (400 MHZ, DMSO-d6) 8 13.36 - 11.54 (broad m, 1H), |
| | 8.60 (s, 1H), 8.45 (s, 2H), 7.91 (s, 1H), 7.65 (s, 2H), 7.33 - 7.19 |
| | (m, 1H), 7.12 (d, J = 7.6 Hz, 2H), 6.40 (s, 1H), 5.35 (d, J = 9.7 |
| | Hz, 1H), 4.95 - 4.80 (m, 2H), 4.59 (d, J = 16.5 Hz, 1H), 4.29 - |
| | 4.09 (m, 1H), 3.92 (s, 1H), 2.49 - 2.41 (overlapped with DMSO, |
| | m, 2H), 2.22 - 1.95 (m, 8H), 1.80 (q, J = 10.3 Hz, 1H), 1.73 - |
| | 1.57 (m, 2H), 1.55 - 1.41 (m, 1H), 1.09 (td, J = 12.6, 5.0 Hz, |
| | 1H), 0.85 - 0.59 (m, 10H). |
| 1487 | ¹H NMR (400 MHz, DMSO-d6) 8 13.24 - 12.78 (bs, 1H), 8.59 |
| | (s, 1H), 8.53 (s, 2H), 7.90 (s, 1H), 7.66 (s, 2H), 7.27 (t, J = 7.4 |
| | Hz, 1H), 7.13 (d, J = 7.4 Hz, 2H), 6.41 (s, 1H), 5.39 - 5.28 (m, |
| | 1H), 4.90 (d, J = 16.5 Hz, 1H), 4.58 (d, J = 16.6 Hz, 1H), 4.27 - |
| | 4.12 (m, 3H), 3.93 - 3.79 (m, 1H), 2.22 - 1.92 (m, 7H), 1.91 - |
| | 1.79 (m, 2H), 1.75 - 1.63 (m, 1H), 1.63 - 1.52 (m, 2H), 1.52 - |
| | 1.41 (m, 2H), 1.35 (t, J = 7.0 Hz, 3H), 0.95 - 0.78 (m, 1H) |
| 1488 | ¹H NMR (400 MHZ, DMSO-d6) 8 13.29 - 11.67 (m, 1H), 8.61 |
| | (s, 1H), 8.54 (s, 2H), 7.91 (s, 1H), 7.66 (s, 2H), 7.26 (t, J = 7.7 |
| | Hz, 1H), 7.12 (d, J = 7.6 Hz, 2H), 6.41 (s, 1H), 5.36 (d, J = 9.9 |
| | Hz, 1H), 4.89 (d, J = 16.5 Hz, 1H), 4.59 (d, J = 16.5 Hz, 1H), |
| | 4.25 - 4.14 (m, 3H), 3.97 - 3.84 (m, 1H), 2.25 - 1.92 (m, 6H), |
| | 1.81 - 1.63 (m, 1H), 1.54 - 1.44 (m, 1H), 1.35 (t, J = 7.0 Hz, |
| | 3H), 1.16 - 1.02 (m, 1H), 0.82 - 0.59 (m, 10H). |
| 1490 | ¹H NMR (400 MHZ, DMSO-d6) 8 13.31 - 11.73 (broad m, 1H), |
| | 8.62 (s, 1H), 8.53 (s, 2H), 7.91 (s, 1H), 7.65 (s, 2H), 7.34 - 7.19 |
| | (m, 1H), 7.20 - 7.02 (m, 2H), 6.40 (s, 1H), 5.34 (d, J = 9.5 Hz, |
| | 1H), 4.82 (d, J = 16.6 Hz, 1H), 4.56 (d, J = 16.7 Hz, 1H), 4.47 - |
| | 4.34 (m, 1H), 4.21 (q, J = 7.0 Hz, 3H), 2.30 - 1.84 (m, 7H), |
| | 1.71 (dd, J = 16.5, 9.7 Hz, 1H), 1.35 (t, J = 7.0 Hz, 3H), 0.95 - |
| | 0.71 (m, 3H), 0.68 - 0.50 (m, 1H). |
| 1498 | ¹H NMR (400 MHZ, DMSO-d6) 8 12.94 (s, 1H), 8.76 (t, J = 1.9 |
| | Hz, 1H), 8.18 (s, 2H), 7.93 (d, J = 7.6 Hz, 1H), 7.62 (d, J = |
| | 18.1 Hz, 2H), 7.24 (s, 1H), 7.13 (d, J = 7.8 Hz, 2H), 6.25 (d, J = |
| | 48.5 Hz, 1H), 5.57 (dtt, J = 57.5, 6.0, 3.2 Hz, 1H), 5.41 (d, J = |
| | 8.1 Hz, 1H), 4.89 (d, J = 16.3 Hz, 1H), 4.59 (d, J = 16.4 Hz, |
| | 1H), 4.34 (dddd, J = 20.9, 9.7, 5.8, 1.3 Hz, 2H), 4.17 (q, J = |
| | 5.2 Hz, 1H), 4.11 (ddd, J = 9.6, 3.2, 1.3 Hz, 1H), 4.05 (ddd, J = |
| | 9.6, 3.2, 1.2 Hz, 1H), 3.23 (d, J = 5.1 Hz, 1H), 2.00 (d, J = |
| | 27.0 Hz, 6H), 1.79 (dd, J = 14.9, 8.7 Hz, 1H), 1.45 (d, J = 14.9 |
| | Hz, 1H), 0.61 (s, 9H). |
| 1499 | ¹H NMR (400 MHZ, DMSO-d6) § 12.80 (s, 1H), 8.52 (s, 1H), |
| | 7.86 (s, 1H), 7.83 (s, 2H), 7.62 (s, 2H), 7.22 (t, J = 7.3 Hz, 1H), |
| | 7.09 (d, J = 7.6 Hz, 2H), 6.29 (s, 1H), 5.43 (d, J = 9.9 Hz, 1H), |
| | 4.73 (d, J = 15.5 Hz, 1H), 4.34 (d, J = 15.5 Hz, 1H), 4.20 (d, J = |
| | 5.2 Hz, 1H), 3.98 (d, J = 10.5 Hz, 1H), 3.57 - 3.44 (m, 2H), |
| | 2.39 - 2.29 (m, 1H), 2.00 (s, 6H), 1.89 (td, J = 12.6, 10.2, 6.6 |
| | Hz, 6H), 1.25 - 1.10 (m, 2H), 0.82 (d, J = 6.9 Hz, 3H), 0.73 (d, |
| | J = 6.8 Hz, 3H), 0.38 (td, J = 8.2, 7.7, 4.5 Hz, 2H), 0.18 (dt, J = |
| | 8.7, 5.3 Hz, 1H), -0.01 (dq, J = 9.3, 4.6 Hz, 1H). |
| 1500 | ¹H NMR (400 MHZ, DMSO-d6) & 13.06 (s, 1H), 8.70 (s, 1H), |
| | 8.32 (s, 2H), 7.94 (s, 1H), 7.66 (s, 2H), 7.25 (s, 1H), 7.18 - 7.00 |
| | (m, 2H), 6.41 (s, 1H), 5.39 (d, J = 9.2 Hz, 1H), 4.85 (d, J = 16.4 |
| | Hz, 1H), 4.62 (d, J = 16.5 Hz, 1H), 4.31 - 4.07 (m, 6H), 2.33 - |
| | 1.84 (m, 6H), 1.79 (dd, J = 14.5, 9.7 Hz, 1H), 1.40 (d, J = 14.9 |
| | Hz, 1H), 0.57 (s, 9H). |
| 1501 | ¹H NMR (400 MHZ, DMSO-d6) 8 12.68 (s, 1H), 8.60 (s, 1H), |
| | 7.95 (d, J = 7.6 Hz, 1H), 7.72 (dt, J = 15.2, 7.7 Hz, 2H), 7.26 |
| | (t, J = 7.6 Hz, 1H), 7.13 (d, J = 7.7 Hz, 2H), 6.80 (s, 2H), 6.35 |
| | (s, 1H), 5.31 (s, 1H), 4.94 (d, J = 16.2 Hz, 1H), 4.60 (s, 1H), |
| | 4.32 (t, J = 11.2 Hz, 1H), 4.18 - 4.01 (m, 2H), 3.72 (s, 2H), 3.17 |
| | (s, 2H), 2.04 (s, 8H), 1.88 (ddt, J = 19.0, 14.3, 6.8 Hz, 2H), 1.14 |
| | (ddt, J = 15.9, 8.0, 4.2 Hz, 1H), 1.06 (tt, J = 8.2, 4.0 Hz, 1H), |
| | 0.58 (s, 1H), 0.48 (s, 1H), 0.38 (ddt, J = 23.7, 13.7, 5.5 Hz, 3H), |
| | 0.19 (dtd, J = 22.2, 9.4, 8.9, 4.7 Hz, 2H), -0.03 (dq, J = 9.1, 4.0 |
| | Hz, 1H), -0.57 (dq, J = 9.6, 4.8 Hz, 1H). |
| 1502 | ¹H NMR (400 MHZ, DMSO-d6) 8 12.98 (s, 1H), 8.61 (d, J = |
| | 2.0 Hz, 1H), 7.91 (d, J = 7.9 Hz, 2H), 7.85 (s, 1H), 7.67 (d, J = |
| | 6.2 Hz, 2H), 7.25 (t, J = 7.6 Hz, 1H), 7.12 (d, J = 7.6 Hz, 2H), |
| | 6.34 (s, 1H), 5.48 (dd, J = 10.9, 4.1 Hz, 1H), 4.80 (d, J = 15.9 |
| | Hz, 1H), 4.39 (d, J = 15.8 Hz, 1H), 4.29 (t, J = 11.2 Hz, 1H), |
| | 4.15 (t, J = 6.8 Hz, 1H), 4.02 (tt, J = 11.1, 4.0 Hz, 1H), 3.63 |
| | (ddd, J = 10.6, 8.2, 2.1 Hz, 1H), 2.17 - 1.94 (m, 8H), 1.91 - 1.74 |
| | (m, 3H), 1.17 (ddd, J = 14.7, 7.8, 3.7 Hz, 1H), 1.06 (tq, J = 8.2, |
| | 5.4 Hz, 1H), 0.55 - 0.24 (m, 6H), 0.18 (dp, J = 13.3, 4.4 Hz, |
| | 2H), 0.01 (dq, J = 9.2, 4.4 Hz, 1H), -0.53 (dq, J = 9.7, 4.9 Hz, |
| | 1H). |
| 1503 | ¹H NMR (400 MHZ, DMSO-d6) & 13.02 (s, 1H), 8.69 (s, 1H), |
| | 8.25 (s, 1H), 8.12 (s, 1H), 7.95 (d, J = 7.4 Hz, 1H), 7.67 (q, J = |
| | 8.5, 8.0 Hz, 2H), 7.27 (t, J = 7.6 Hz, 1H), 7.13 (d, J = 7.7 Hz, |
| | 2H), 6.42 (s, 1H), 5.39 (dt, J = 11.0, 3.4 Hz, 1H), 4.83 (dd, J = |
| | 16.4, 14.0 Hz, 1H), 4.55 (dd, J = 30.4, 16.4 Hz, 1H), 4.19 - 3.97 |
| | (m, 5H), 3.97 - 3.81 (m, 1H), 2.05 (d, J = 48.7 Hz, 6H), 1.78 |
| | (ddd, J = 14.0, 8.8, 4.9 Hz, 1H), 1.62 (d, J = 22.2 Hz, 2H), 1.48 - |
| | 1.34 (m, 2H), 0.57 (s, 9H). |
| 1504 | ¹H NMR (400 MHZ, DMSO-d6) & 13.02 (s, 1H), 8.70 (s, 1H), |
| | 8.09 (s, 2H), 7.93 (s, 1H), 7.65 (s, 2H), 7.25 (s, 1H), 7.12 (d, J = |
| | 7.4 Hz, 2H), 6.39 (s, 1H), 5.38 (d, J = 8.6 Hz, 1H), 4.85 (d, J = |
| | 16.2 Hz, 1H), 4.54 (d, J = 16.3 Hz, 1H), 4.08 - 3.98 (m, 2H), |
| | 3.47 (t, J = 6.8 Hz, 6H), 3.23 (s, 1H), 1.92 (t, J = 6.8 Hz, 7H), |
| | 1.79 (dd, J = 15.1, 8.2 Hz, 1H), 1.39 (d, J = 15.0 Hz, 1H), 0.69 - |
| | 0.49 (m, 11H). |
| 1505 | ¹H NMR (400 MHZ, DMSO-d6) 8 12.80 (s, 1H), 8.61 (s, 1H), |
| | 8.27 (s, 1H), 8.06 (s, 1H), 7.92 (t, J = 4.7 Hz, 1H), 7.68 (d, J = |
| | 4.6 Hz, 2H), 7.27 (t, J = 7.6 Hz, 1H), 7.14 (d, J = 7.7 Hz, 2H), |
| | 6.37 - 6.29 (m, 1H), 5.35 (dd, J = 10.9, 3.8 Hz, 1H), 4.95 (dd, |
| | J = 16.3, 8.3 Hz, 1H), 4.48 (dd, J = 22.3, 16.3 Hz, 1H), 4.10 |
| | (td, J = 11.1, 8.2 Hz, 1H), 4.06 - 3.95 (m, 1H), 3.66 (s, 2H), |
| | 3.61 (s, 1H), 3.18 (s, 1H), 2.05 (s, 6H), 1.89 (ddt, J = 14.7, 10.0, |
| | 4.6 Hz, 1H), 1.30 (s, 4H), 1.15 (ddd, J = 14.4, 7.7, 3.2 Hz, 1H), |
| | 1.03 (s, 2H), 0.47 (t, J = 7.3 Hz, 1H), 0.39 (dq, J = 8.9, 4.5 Hz, |
| | 1H), 0.19 (dtt, J = 9.1, 6.4, 3.1 Hz, 1H), 0.01 (dq, J = 9.5, 4.7 |
| | Hz, 1H), -0.54 (dq, J = 9.5, 4.7 Hz, 1H). |
| 1508 | ¹H NMR (400 MHZ, DMSO-d6) 8 13.07 (br. s., 1H), 8.61 (s, |
| | 1H), 7.94 (br. s., 1H), 7.65 (br. s., 2H), 7.51 - 7.42 (m, 1H), 7.25 |
| | (br. s., 1H), 7.13 (s, 2H), 6.59 (d, J = 5.6 Hz, 1H), 6.50 (d, J = |
| | 7.8 Hz, 1H), 6.38 (br. s., 1H), 5.39 - 5.24 (m, 1H), 4.91 - 4.80 |
| | (m, 1H), 4.69 (d, J = 15.4 Hz, 1H), 4.41 - 4.23 (m, 3H), 3.46 |
| | (br. s., 4H), 3.27 - 3.20 (m, 3H), 2.25 - 1.90 (m, 7H), 1.80 - 1.66 |
| | (m, 1H), 1.25 - 1.06 (m, 9H), 0.82 (d, J = 21.5 Hz, 3H). |
| 1509 | ¹H NMR (400 MHZ, DMSO-d6, 80°C) 8 12.34 (br. s, 1H), 8.70 |
| | (s, 1H), 8.42 (s, 2H), 7.95 (br. d, J = 7.8 Hz, 1H), 7.71 - 7.58 |
| | (m, 2H), 7.27 - 7.21 (m, 1H), 7.11 (d, J = 7.6 Hz, 2H), 6.29 (br. |
| | s, 1H), 5.39 (dd, J = 10.6, 4.3 Hz, 1H), 4.98 (d, J = 16.6 Hz, |
| | 1H), 4.88 (quin, J = 6.8 Hz, 1H), 4.57 (d, J = 16.6 Hz, 1H), |
| | 4.29 - 4.19 (m, 1H), 4.18 - 4.08 (m, 1H), 2.49 - 2.44 (m, 2H), |
| | 2.30 - 2.17 (m, 1H), 2.16 - 1.98 (m, 8H), 1.90 - 1.60 (m, 3H), |
| | 1.14 (d, J = 20.8 Hz, 3H), 0.93 (d, J = 20.8 Hz, 3H). |
| 1510 | ¹H NMR (400 MHZ, DMSO-d6) 8 13.07 (br. s., 1H), 8.69 (s, |
| | 1H), 7.95 (br. s., 1H), 7.70 (br. s., 2H), 7.43 (dd, J = 8.3, 7.3 |
| | Hz, 1H), 7.29 - 7.20 (m, 1H), 7.12 (d, J = 6.8 Hz, 2H), 6.59 (d, |
| | J = 7.3 Hz, 1H), 6.42 (br. s., 1H), 6.34 (d, J = 8.6 Hz, 1H), 5.22 |
| | (d, J = 6.1 Hz, 1H), 4.76 (d, J = 15.7 Hz, 1H), 4.34 (d, J = 15.7 |
| | Hz, 1H), 4.18 - 4.08 (m, 2H), 4.00 (d, J = 9.0 Hz, 1H), 3.53 (d, |
| | J = 9.0 Hz, 1H), 3.48 - 3.34 (m, 2H), 3.01 (s, 3H), 2.23 - 2.13 |
| | (m, 2H), 2.05 - 1.78 (m, 8H), 1.72 - 1.62 (m, 1H), 1.51 (s, 3H), |
| | 1.37 (d, J = 14.9 Hz, 1H), 0.53 (s, 9H). |
| 1511 | ¹H NMR (400 MHZ, DMSO-d6) 8 13.07 (br. s., 1H), 8.65 (br. |
| | s., 1H), 8.01 - 7.89 (m, 1H), 7.75 - 7.60 (m, 2H), 7.46 - 7.39 (m, |
| | 1H), 7.31 - 7.21 (m, 1H), 7.16 - 7.07 (m, 2H), 6.59 (d, J = 7.3 |
| | Hz, 1H), 6.48 - 6.39 (m, 1H), 6.36 (d, J = 8.6 Hz, 1H), 5.28 (dd, |
| | J = 10.4, 4.0 Hz, 1H), 4.70 (d, J = 15.6 Hz, 1H), 4.35 (d, J = |
| | 15.4 Hz, 1H), 4.22 (t, J = 10.8 Hz, 1H), 4.12 - 4.04 (m, 1H), |
| | 3.92 (d, J = 8.8 Hz, 1H), 3.74 (d, J = 9.0 Hz, 1H), 3.50 - 3.35 |
| | (m, 2H), 3.19 (s, 3H), 2.24 - 2.15 (m, 2H), 2.07 - 1.77 (m, 8H), |
| | 1.73 - 1.64 (m, 1H), 1.45 (s, 3H), 1.38 (d, J = 14.9 Hz, 1H), |
| | 0.53 (s, 9H). |
| 1513 | ¹H NMR (400 MHz, DMSO-d6) 8 13.06 (s, 1H), 10.31 (s, 1H), |
| | 8.94 (s, 2H), 8.72 (s, 1H), 7.95 (s, 1H), 7.67 (s, 2H), 7.26 (t, J = |
| | 7.7 Hz, 1H), 7.13 (d, J = 7.7 Hz, 2H), 6.41 (s, 1H), 5.42 (d, J = |
| | 10.2 Hz, 1H), 4.87 (d, J = 16.7 Hz, 1H), 4.69 (d, J = 16.8 Hz, |
| | 1H), 4.18 (t, J = 11.2 Hz, 1H), 4.00 (s, 1H), 2.10 (s, 9H), 1.82 - |
| | 1.75 (m, 1H), 1.41 (d, J = 14.6 Hz, 1H), 0.58 (s, 9H). |
| 1514 | ¹H NMR (400 MHZ, DMSO-d6) 8 13.15 (s, 1H), 10.02 (s, 1H), |
| | 8.83 (s, 2H), 8.71 (s, 1H), 7.95 (d, J = 7.4 Hz, 1H), 7.67 (q, J = |
| | 9.6, 8.5 Hz, 2H), 7.26 (t, J = 7.7 Hz, 1H), 7.19 (s, 2H), 6.41 (s, |
| | 1H), 5.42 (dd, J = 11.3, 4.1 Hz, 1H), 4.86 (d, J = 16.7 Hz, 1H), |
| | 4.68 (d, J = 16.8 Hz, 1H), 4.18 (t, J = 11.2 Hz, 1H), 4.01 (d, J = |
| | 10.9 Hz, 1H), 3.72 (d, J = 2.1 Hz, 3H), 2.00 (s, 6H), 1.77 (dd, |
| | J = 15.2, 8.9 Hz, 1H), 1.41 (d, J = 14.6 Hz, 1H), 0.58 (s, 9H). |
| 1515 | ¹H NMR (400 MHZ, DMSO-d6) 8 13.06 (s, 1H), 9.94 (s, 1H), |
| | 8.83 (t, J = 1.8 Hz, 2H), 8.71 (s, 1H), 7.94 (s, 1H), 7.66 (s, 2H), |
| | 7.26 (s, 1H), 7.13 (s, 2H), 6.41 (s, 1H), 5.41 (d, J = 10.5 Hz, |
| | 1H), 5.03 - 4.84 (m, 2H), 4.67 (d, J = 16.7 Hz, 1H), 4.17 (t, J = |
| | 11.1 Hz, 1H), 4.00 (s, 1H), 2.03 (d, J = 67.9 Hz, 6H), 1.77 (dd, |
| | J = 15.1, 9.0 Hz, 1H), 1.43 (s, 1H), 1.27 (dt, J = 6.3, 1.8 Hz, |
| | 6H), 0.74 - 0.45 (m, 9H). |
| 1516 | ¹H NMR (400 MHZ, DMSO-d6) 8 13.07 (s, 1H), 8.89 (s, 2H), |
| | 8.72 (s, 1H), 7.96 (d, J = 7.3 Hz, 1H), 7.68 (q, J = 8.5, 8.0 Hz, |
| | 2H), 7.26 (t, J = 7.8 Hz, 1H), 7.13 (d, J = 7.6 Hz, 2H), 6.43 (s, |
| | 1H), 5.41 (dd, J = 11.0, 4.1 Hz, 1H), 4.91 (d, J = 16.8 Hz, 1H), |
| | 4.78 (d, J = 17.4 Hz, 1H), 4.26 (t, J = 11.2 Hz, 1H), 4.07 (d, J = |
| | 10.7 Hz, 1H), 3.20 (s, 3H), 2.26 - 1.75 (m, 10H), 1.42 (d, J = |
| | 15.0 Hz, 1H), 0.57 (d, J = 2.4 Hz, 9H). |
| 1517 | ¹H NMR (400 MHz, DMSO-d6) 8 13.09 (s, 1H), 8.84 (s, 2H), |
| | 8.72 (s, 1H), 7.96 (s, 1H), 7.68 (s, 2H), 7.26 (t, J = 7.4 Hz, 1H), |
| | 7.13 (d, J = 7.7 Hz, 2H), 6.43 (s, 1H), 5.52 - 5.36 (m, 1H), 4.88 |
| | (d, J = 16.8 Hz, 1H), 4.76 (d, J = 16.9 Hz, 1H), 4.26 (t, J = 11.1 |
| | Hz, 1H), 4.06 (q, J = 8.5, 6.2 Hz, 1H), 3.67 (s, 3H), 3.29 (s, |
| | 3H), 2.03 (d, J = 64.9 Hz, 6H), 1.78 (dd, J = 15.2, 8.8 Hz, 1H), |
| | 1.42 (d, J = 14.8 Hz, 1H), 0.57 (s, 9H). |
| 1518 | ¹H NMR (400 MHZ, DMSO-d6) 8 13.07 (s, 1H), 8.90 - 8.78 (m, |
| | 2H), 8.71 (s, 1H), 7.95 (s, 1H), 7.67 (s, 2H), 7.25 (d, J = 8.2 |
| | Hz, 1H), 7.13 (d, J = 7.6 Hz, 2H), 6.42 (s, 1H), 5.40 (s, 1H), |
| | 4.93 - 4.81 (m, 2H), 4.76 (d, J = 16.7 Hz, 1H), 4.25 (t, J = 11.3 |
| | Hz, 1H), 4.04 (s, 1H), 3.30 (s, 3H), 2.02 (d, J = 70.8 Hz, 6H), |
| | 1.78 (t, J = 12.2 Hz, 1H), 1.41 (d, J = 14.8 Hz, 1H), 1.21 (d, J = |
| | 5.8 Hz, 6H), 0.70 - 0.41 (m, 9H). |
| 1520 | ¹H NMR (400 MHZ, DMSO-d6) 8 13.34 - 11.33 (broad m, 1H), |
| | 8.69 (s, 1H), 8.02 (s, 2H), 7.95 (s, 1H), 7.66 (s, 2H), 7.32 - 7.19 |
| | (m, 1H), 7.12 (d, J = 7.7 Hz, 2H), 6.41 (s, 1H), 5.41 (d, J = 8.6 |
| | Hz, 1H), 4.83 (d, J = 16.4 Hz, 1H), 4.65 (d, J = 16.5 Hz, 1H), |
| | 4.28 - 4.07 (m, 2H), 3.64 (s, 4H), 2.22 - 1.86 (m, 7H), 1.78 (d, |
| | J = 15.6 Hz, 1H), 1.29 (s, 6H), 0.94 (s, 3H), 0.71 (s, 3H). |
| 1521 | ¹H NMR (400 MHZ, DMSO) & 8.69 (s, 1H), 8.54 (s, 2H), 8.03 - |
| | 7.87 (m, 1H), 7.76 - 7.56 (m, 2H), 7.31 - 7.20 (m, 1H), 7.19 - |
| | 7.06 (m, 2H), 6.42 (s, 1H), 5.39 (d, J = 10.0 Hz, 1H), 4.86 (d, |
| | J = 16.5 Hz, 1H), 4.66 (d, J = 16.5 Hz, 1H), 4.19 (t, J = 11.2 |
| | Hz, 1H), 4.11 (t, J = 6.5 Hz, 2H), 4.08 - 4.00 (m, 1H), 2.26 - |
| | 1.84 (m, 6H), 1.83 - 1.67 (m, 3H), 1.40 (d, J = 14.9 Hz, 1H), |
| | 0.98 (t, J = 7.4 Hz, 3H), 0.57 (s, 9H). |
| 1522 | ¹H NMR (400 MHZ, DMSO) 8 8.59 (s, 1H), 8.54 (s, 2H), 7.98 - |
| | 7.85 (m, 1H), 7.73 - 7.60 (m, 2H), 7.27 (t, J = 7.6 Hz, 1H), |
| | 7.13 (d, J = 7.6 Hz, 2H), 6.42 (s, 1H), 5.34 (dd, J = 10.8, 4.3 |
| | Hz, 1H), 4.90 (d, J = 16.5 Hz, 1H), 4.59 (d, J = 16.5 Hz, 1H), |
| | 4.18 (t, J = 11.2 Hz, 1H), 4.11 (t, J = 6.5 Hz, 2H), 3.92 - 3.82 |
| | (m, 1H), 2.15 - 1.95 (m, 8H), 1.92 - 1.81 (m, 2H), 1.80 - 1.64 |
| | (m, 3H), 1.62 - 1.40 (m, 3H), 0.98 (t, J = 7.4 Hz, 3H), 0.93 - |
| | 0.82 (m, 1H). |
| 1523 | ¹H NMR (400 MHZ, DMSO) 8 8.63 (s, 1H), 8.54 (s, 2H), 7.98 - |
| | 7.89 (m, 1H), 7.66 (s, 2H), 7.26 (t, J = 7.6 Hz, 1H), 7.13 (d, J = |
| | 7.6 Hz, 2H), 6.39 (s, 1H), 5.36 (dd, J = 10.8, 4.2 Hz, 1H), |
| | 4.90 (d, J = 16.5 Hz, 1H), 4.63 (d, J = 16.5 Hz, 1H), 4.19 (t, J = |
| | 11.1 Hz, 1H), 4.11 (t, J = 6.5 Hz, 2H), 4.06 - 3.95 (m, 1H), |
| | 2.16 - 1.86 (m, 6H), 1.82 - 1.66 (m, 3H), 1.47 - 1.33 (m, 1H), |
| | 1.21 (ddd, J = 13.7, 10.2, 3.0 Hz, 1H), 0.98 (t, J = 7.4 Hz, 3H), |
| | 0.75 (d, J = 6.6 Hz, 3H), 0.28 (d, J = 6.4 Hz, 3H). |
| 1524 | ¹H NMR (400 MHZ, DMSO-d6) & 13.46 - 11.55 (broad m, 1H), |
| | 8.66 (s, 1H), 8.53 (s, 2H), 7.93 (s, 1H), 7.67 (s, 2H), 7.32 - 7.20 |
| | (m, 1H), 7.12 (d, J = 7.6 Hz, 2H), 6.39 (s, 1H), 5.35 (d, J = 9.2 |
| | Hz, 1H), 4.84 (d, J = 16.5 Hz, 1H), 4.53 (d, J = 16.6 Hz, 1H), |
| | 4.32 - 4.12 (m, 4H), 2.28 - 1.85 (m, 6H), 1.61 (d, J = 14.9 Hz, |
| | 1H), 1.48 (dd, J = 15.2, 9.6 Hz, 1H), 1.35 (t, J = 6.9 Hz, 3H), |
| | 0.37 (s, 3H), 0.27 (dt, J = 9.4, 4.8 Hz, 1H), 0.16 (dt, J = 9.5, |
| | 4.8 Hz, 1H), 0.09 - 0.02 (m, 1H), -0.01 - - 0.10 (m, 1H). |
| 1306 | ¹H NMR (400 MHz, DMSO-d6) 8 13.71 - 11.37 (Broad m, 1H), |
| | 8.65 (s, 1H), 8.52 (s, 2H), 7.93 (s, 1H), 7.67 (s, 2H), 7.31 - 7.19 |
| | (m, 1H), 7.12 (d, J = 7.6 Hz, 2H), 6.40 (s, 1H), 5.35 (d, J = 9.8 |
| | Hz, 1H), 4.88 - 4.74 (m, 2H), 4.52 (d, J = 16.6 Hz, 1H), 4.32 - |
| | 4.11 (m, 2H), 2.20 - 1.88 (m, 6H), 1.61 (d, J = 14.8 Hz, 1H), |
| | 1.47 (dd, J = 15.1, 9.4 Hz, 1H), 1.30 (dd, J = 6.0, 2.4 Hz, 6H), |
| | 0.37 (s, 3H), 0.27 (dt, J = 9.5, 4.7 Hz, 1H), 0.16 (dt, J = 9.5, |
| | 4.7 Hz, 1H), 0.06 (d, J = 4.5 Hz, 1H), -0.02 - - 0.10 (overlapped |
| | with TMS, m, 1H). |
| 1307 | ¹H NMR (400 MHZ, DMSO-d6) 8 13.05 (s, 1H), 8.65 (s, 1H), |
| | 8.03 (s, 2H), 7.97 - 7.84 (m, 1H), 7.67 (s, 2H), 7.25 (t, J = 7.7 |
| | Hz, 1H), 7.12 (d, J = 7.7 Hz, 2H), 6.39 (s, 1H), 5.40 - 5.28 (m, |
| | 1H), 4.81 (d, J = 16.3 Hz, 1H), 4.43 (d, J = 16.4 Hz, 1H), 4.21 |
| | (t, J = 10.4 Hz, 1H), 4.11 (t, J = 11.1 Hz, 1H), 3.64 (s, 4H), |
| | 1.96 (s, 6H), 1.60 (d, J = 14.8 Hz, 1H), 1.47 (dd, J = 15.2, 9.8 |
| | Hz, 1H), 1.29 (s, 6H), 0.37 (s, 3H), 0.27 (dt, J = 9.6, 4.5 Hz, |
| | 1H), 0.16 (dt, J = 9.3, 4.7 Hz, 1H), 0.05 (dt, J = 9.3, 4.6 Hz, |
| | 1H), -0.01 - - 0.10 (m, 1H). |
| 1308 | ¹H NMR (400 MHz, DMSO-d6) 8 13.07 (br. s., 1H), 8.66 (br. |
| | s., 1H), 8.52 (s, 2H), 7.98 - 7.87 (m, 1H), 7.74 - 7.50 (m, 2H), |
| | 7.24 (br. s., 1H), 7.18 - 7.05 (m, 2H), 6.33 (br. s., 1H), 5.43 - |
| | 5.31 (m, 1H), 4.88 (d, J = 16.6 Hz, 1H), 4.81 (dt, J = 12.1, 5.9 |
| | Hz, 1H), 4.63 (d, J = 16.6 Hz, 1H), 4.23 (br. s., 2H), 2.29 - 1.85 |
| | (m, 7H), 1.82 - 1.69 (m, 1H), 1.30 (dd, J = 6.0, 2.1 Hz, 6H), |
| | 1.16 - 1.06 (m, 3H), 0.93 - 0.82 (m, 3H). |
| 1525 | ¹H NMR (400 MHZ, DMSO) 8 13.03 (s, 1H), 8.59 (s, 1H), 8.03 |
| | (s, 2H), 7.90 (s, 1H), 7.65 (s, 2H), 7.25 (s, 1H), 7.13 (s, 2H), |
| | 6.39 (s, 1H), 5.34 (d, J = 10.7 Hz, 1H), 4.87 (d, J = 16.3 Hz, |
| | 1H), 4.47 (d, J = 16.2 Hz, 1H), 4.11 (t, J = 12.8 Hz, 1H), 3.89 |
| | (s, 1H), 3.64 (s, 4H), 2.02 (s, 7H), 1.71 (d, J = 12.1 Hz, 1H), |
| | 1.48 (s, 1H), 1.29 (s, 6H), 1.16 - 1.04 (m, 1H), 0.72 (s, 9H). |
| 1309 | ¹H NMR (400 MHz, DMSO) § 12.47 (d, J = 335.8 Hz, 1H), |
| | 8.60 (s, 1H), 8.03 (s, 2H), 7.89 (s, 1H), 7.63 (s, 2H), 7.24 (s, |
| | 1H), 7.11 (s, 2H), 6.35 (s, 1H), 5.32 (s, 1H), 4.90 (d, J = 16.3 |
| | Hz, 1H), 4.46 (d, J = 16.2 Hz, 1H), 4.07 (s, 1H), 3.93 (s, 1H), |
| | 3.64 (s, 4H), 2.01 (s, 4H), 1.73 (s, 1H), 1.51 (s, 1H), 1.29 (s, |
| | 6H), 1.16 - 0.96 (m, 3H), 0.90 - 0.78 (m, 2H), 0.71 (d, J = 6.4 |
| | Hz, 3H), 0.64 (d, J = 6.4 Hz, 3H). |
| 1526 | ¹H NMR (400 MHZ, DMSO-d6) 8 13.25 - 12.71 (bs, 1H), 8.61 |
| | (s, 1H), 8.55 (s, 2H), 7.94 (d, J = 7.6 Hz, 1H), 7.74 - 7.54 (m, |
| | 2H), 7.26 (t, J = 7.6 Hz, 1H), 7.12 (d, J = 7.6 Hz, 2H), 6.33 (s, |
| | 1H), 5.89 - 5.78 (m, 1H), 4.86 - 4.74 (m, 2H), 4.61 (d, J = 16.8 |
| | Hz, 1H), 4.05 - 3.95 (m, 1H), 2.12 (s, 3H), 1.91 (s, 3H), 1.84 |
| | (dd, J = 15.2, 9.2 Hz, 1H), 1.45 (d, J = 14.9 Hz, 1H), 1.38 (d, J = |
| | 6.5 Hz, 3H), 1.31 (d, J = 4.0 Hz, 3H), 1.29 (d, J = 4.0 Hz, |
| | 3H), 0.59 (s, 9H) |
| 1527 | ¹H NMR (400 MHZ, DMSO-d6) 8 13.27 - 12.62 (bs, 1H), 8.90 |
| | (s, 1H), 8.52 (s, 2H), 7.95 (d, J = 7.3 Hz, 1H), 7.77 - 7.63 (m, |
| | 2H), 7.26 (t, J = 7.6 Hz, 1H), 7.13 (d, J = 7.7 Hz, 2H), 6.40 (s, |
| | 1H), 5.03 (t, J = 9.8 Hz, 1H), 4.96 - 4.84 (m, 1H), 4.83 - 4.71 |
| | (m, 1H), 4.66 (d, J = 16.3 Hz, 1H), 4.58 (d, J = 16.3 Hz, 1H), |
| | 2.44 (d, J = 7.1 Hz, 3H), 2.23 - 1.86 (bs, 6H), 1.79 (dd, J = |
| | 15.4, 9.2 Hz, 1H), 1.38 (d, J = 15.1 Hz, 1H), 1.30 (d, J = 3.5 |
| | Hz, 3H), 1.29 (d, J = 3.6 Hz, 3H), 0.45 (s, 9H) |
| 1528 | ¹H NMR (400 MHZ, DMSO-d6) 8 13.26 - 11.61 (broad m, 1H), |
| | 8.61 (s, 1H), 8.53 (s, 2H), 7.91 (s, 1H), 7.65 (s, 2H), 7.31 - 7.21 |
| | (m, 1H), 7.12 (d, J = 7.6 Hz, 2H), 6.38 (s, 1H), 5.36 (d, J = 10.2 |
| | Hz, 1H), 4.90 (d, J = 16.5 Hz, 1H), 4.67 - 4.52 (m, 2H), 4.19 (t, |
| | J = 11.2 Hz, 1H), 4.04 - 3.87 (m, 1H), 2.23 - 1.89 (m, 6H), 1.82 - |
| | 1.71 (m, 1H), 1.70 - 1.56 (m, 2H), 1.56 - 1.43 (m, 1H), 1.26 |
| | (d, J = 6.0 Hz, 3H), 1.18 - 1.07 (m, 1H), 1.07 - 0.96 (m, 1H), |
| | 0.93 (t, J = 7.4 Hz, 3H), 0.85 (q, J = 6.1 Hz, 1H), 0.71 (d, J = |
| | 6.5 Hz, 3H), 0.64 (d, J = 6.4 Hz, 3H). |
| 1529 | ¹H NMR (400 MHZ, DMSO-d6) 8 13.47 - 11.51 (broad m, 1H), |
| | 8.61 (s, 1H), 8.02 (s, 2H), 7.90 (s, 1H), 7.65 (s, 2H), 7.34 - 7.19 |
| | (m, 1H), 7.12 (d, J = 7.8 Hz, 2H), 6.39 (s, 1H), 5.43 - 5.22 (m, |
| | 1H), 4.78 (d, J = 16.4 Hz, 1H), 4.46 (d, J = 16.5 Hz, 1H), 4.42 - |
| | 4.30 (m, 1H), 4.14 (t, J = 11.2 Hz, 1H), 3.64 (s, 4H), 2.31 - |
| | 1.80 (m, 7H), 1.71 (dd, J = 16.5, 9.7 Hz, 1H), 1.29 (s, 6H), 0.87 - |
| | 0.72 (m, 3H), 0.66 - 0.53 (m, 1H). |
| 1534 | ¹H NMR (400 MHZ, DMSO-d6) 8 13.05 (s, 1H), 8.69 (s, 1H), |
| | 7.93 (s, 1H), 7.91 (s, 1H), 7.86 (d, J = 1.8 Hz, 1H), 7.68 (s, 2H), |
| | 7.25 (s, 1H), 7.12 (s, 2H), 6.42 (s, 1H), 5.32 (s, 1H), 4.79 (d, J = |
| | 15.8 Hz, 1H), 4.41 (d, J = 16.2 Hz, 1H), 4.25 (s, 1H), 4.07 |
| | (s, 2H), 3.79 (d, J = 11.1 Hz, 1H), 3.68 (d, J = 10.4 Hz, 1H), |
| | 3.65 - 3.57 (m, 1H), 3.51 (d, J = 9.1 Hz, 1H), 3.10 (dd, J = 21.2, |
| | 10.5 Hz, 2H), 2.18 - 1.72 (m, 12H), 1.42 (d, J = 15.1 Hz, 1H), |
| | 1.30 (s, 2H), 1.24 (s, 2H), 0.71 - 0.35 (s, 9H). |
| 1535 | ¹H NMR (400 MHZ, DMSO-d6) 8 12.85 (s, 1H), 8.63 (s, 1H), |
| | 7.93 (d, J = 7.3 Hz, 1H), 7.86 (s, 2H), 7.66 (d, J = 8.3 Hz, 2H), |
| | 7.25 (t, J = 7.6 Hz, 1H), 7.12 (d, J = 7.3 Hz, 2H), 6.42 (s, 1H), |
| | 5.51 (dd, J = 10.9, 4.2 Hz, 1H), 4.64 (d, J = 15.5 Hz, 1H), 4.50 - |
| | 4.36 (m, 2H), 4.28 (s, 1H), 4.01 (s, 1H), 3.83 (d, J = 11.3 Hz, |
| | 1H), 3.68 (d, J = 9.7 Hz, 1H), 3.57 - 3.50 (m, 1H), 3.44 (d, J = |
| | 9.0 Hz, 1H), 3.21 - 3.10 (m, 2H), 2.28 (t, J = 7.4 Hz, 1H), 2.01 |
| | (d, J = 51.9 Hz, 10H), 1.73 (dd, J = 15.2, 8.9 Hz, 1H), 1.61 (d, |
| | J = 13.1 Hz, 1H), 1.44 (d, J = 14.9 Hz, 1H), 1.35 (d, J = 3.3 |
| | Hz, 1H), 1.31 (s, 2H), 0.58 (s, 9H). |
| 1536 | ¹H NMR (400 MHZ, DMSO-d6) § 12.98 (s, 1H), 8.72 (s, 1H), |
| | 7.94 (s, 1H), 7.91 (s, 1H), 7.82 (s, 1H), 7.67 (s, 2H), 7.25 (s, |
| | 1H), 7.12 (s, 2H), 6.42 (s, 1H), 5.39 (d, J = 10.5 Hz, 1H), 4.74 |
| | (d, J = 15.9 Hz, 1H), 4.43 (d, J = 15.8 Hz, 1H), 4.26 (s, 1H), |
| | 4.19 (t, J = 6.6 Hz, 1H), 4.06 (s, 1H), 3.65 (dt, J = 11.2, 5.8 Hz, |
| | 1H), 3.48 (t, J = 9.3 Hz, 1H), 1.81 (dddd, J = 61.7, 36.5, 18.2, |
| | 9.3 Hz, 16H), 1.58 (d, J = 8.5 Hz, 1H), 1.42 (d, J = 15.0 Hz, |
| | 1H), 1.23 (s, 1H), 0.56 (s, 9H). |
| 1537 | ¹H NMR (400 MHZ, DMSO-d6) 8 13.01 (s, 1H), 8.67 (s, 1H), |
| | 7.93 (s, 1H), 7.87 (s, 1H), 7.82 (s, 1H), 7.67 (s, 2H), 7.25 (s, |
| | 1H), 7.12 (s, 2H), 6.45 (s, 1H), 5.48 (d, J = 10.3 Hz, 1H), 4.68 |
| | (d, J = 15.8 Hz, 1H), 4.48 - 4.40 (m, 1H), 4.33 (d, J = 21.4 Hz, |
| | 2H), 4.03 (s, 1H), 3.58 (q, J = 6.5, 5.7 Hz, 1H), 2.65 (d, J = 9.1 |
| | Hz, 1H), 2.10 - 1.63 (m, 17H), 1.43 (d, J = 15.0 Hz, 1H), 1.34 |
| | (d, J = 6.8 Hz, 1H), 0.57 (s, 9H). |
| 1540 | ¹H NMR (400 MHZ, DMSO-d6) 8 13.04 (s, 1H), 8.69 (s, 1H), |
| | 7.96 (s, 1H), 7.91 (s, 1H), 7.78 (s, 1H), 7.68 (s, 2H), 7.25 (s, |
| | 1H), 7.12 (s, 2H), 6.41 (s, 1H), 5.29 (d, J = 9.8 Hz, 1H), 4.84 |
| | (d, J = 15.9 Hz, 1H), 4.49 - 4.41 (m, 1H), 4.27 (t, J = 6.2 Hz, |
| | 1H), 4.25 - 4.19 (m, 1H), 4.05 (t, J = 10.0 Hz, 2H), 3.82 (t, J = |
| | 6.8 Hz, 1H), 3.75 (dd, J = 10.6, 6.0 Hz, 1H), 3.49 (dd, J = 4.4 |
| | Hz, 1H), 3.42 (s, 1H), 2.74 - 2.69 (m, 1H), 2.19 - 1.72 (m, 10H), |
| | 1.41 (d, J = 15.1 Hz, 1H), 0.56 (s, 9H). |
| 1541 | ¹H NMR (400 MHZ, DMSO-d6) 8 13.04 (s, 1H), 8.64 (s, 1H), |
| | 7.95 (s, 1H), 7.92 (s, 1H), 7.76 (s, 1H), 7.67 (s, 2H), 7.25 (s, |
| | 1H), 7.12 (s, 2H), 6.43 (d, J = 13.8 Hz, 1H), 5.34 (d, J = 10.4 |
| | Hz, 1H), 4.77 (d, J = 15.9 Hz, 1H), 4.44 (d, J = 15.2 Hz, 1H), |
| | 4.31 (t, J = 6.3 Hz, 1H), 4.24 - 4.18 (m, 1H), 4.13 (d, J = 9.7 |
| | Hz, 1H), 4.03 (s, 1H), 3.84 (t, J = 6.8 Hz, 1H), 3.73 (q, J = 4.6 |
| | Hz, 1H), 3.48 (dt, J = 7.2, 3.8 Hz, 2H), 2.67 (dd, J = 3.8, 2.0 |
| | Hz, 1H), 2.17 - 1.83 (m, 9H), 1.76 (dd, J = 15.3, 8.8 Hz, 1H), |
| | 1.42 (d, J = 14.9 Hz, 1H), 0.55 (s, 9H). |
| 1542 | ¹H NMR (400 MHZ, DMSO-d6) 8 12.93 (s, 1H), 8.72 (s, 1H), |
| | 7.93 (d, J = 6.2 Hz, 1H), 7.90 - 7.78 (m, 2H), 7.67 (s, 2H), 7.24 |
| | (d, J = 7.9 Hz, 1H), 7.12 (d, J = 6.6 Hz, 2H), 6.42 (s, 1H), 5.41 |
| | (d, J = 10.7 Hz, 1H), 4.74 (d, J = 15.9 Hz, 1H), 4.43 (d, J = |
| | 16.0 Hz, 1H), 4.36 - 4.18 (m, 2H), 4.08 (s, 1H), 3.59 (t, J = 7.1 |
| | Hz, 2H), 2.80 (s, 1H), 2.67 (s, 1H), 2.22 - 1.83 (m, 8H), 1.81 - |
| | 1.67 (m, 3H), 1.51 (d, J = 13.9 Hz, 4H), 0.56 (s, 9H). |
| 1543 | ¹H NMR (400 MHZ, DMSO-d6) & 12.78 (s, 1H), 8.71 (s, 1H), |
| | 7.93 (d, J = 7.2 Hz, 1H), 7.83 (d, J = 10.5 Hz, 2H), 7.66 (s, |
| | 2H), 7.25 (t, J = 7.6 Hz, 1H), 7.12 (d, J = 7.7 Hz, 2H), 6.43 (d, |
| | J = 18.8 Hz, 1H), 5.52 (dd, J = 10.8, 4.1 Hz, 1H), 4.70 (d, J = |
| | 15.9 Hz, 1H), 4.50 - 4.41 (m, 1H), 4.33 (td, J = 11.4, 6.5 Hz, |
| | 2H), 4.03 (s, 1H), 3.58 (t, J = 6.9 Hz, 2H), 2.79 (dq, J = 12.2, |
| | 7.9, 6.1 Hz, 1H), 2.03 (ddq, J = 21.1, 14.0, 7.2, 6.2 Hz, 8H), |
| | 1.80 - 1.65 (m, 4H), 1.59 - 1.47 (m, 3H), 1.43 (d, J = 14.9 Hz, |
| | 1H), 0.57 (s, 9H). |
| 1544 | ¹H NMR (400 MHZ, DMSO-d6) 8 12.98 (s, 1H), 8.66 (s, 1H), |
| | 7.94 (d, J = 6.7 Hz, 1H), 7.90 (s, 1H), 7.73 (s, 1H), 7.67 (s, 2H), |
| | 7.25 (t, J = 7.6 Hz, 1H), 7.12 (d, J = 7.7 Hz, 2H), 6.41 (s, 1H), |
| | 5.33 (d, J = 8.1 Hz, 1H), 4.76 (d, J = 15.8 Hz, 1H), 4.42 (d, J = |
| | 16.0 Hz, 1H), 4.29 (dt, J = 8.3, 5.7 Hz, 1H), 4.20 (t, J = 10.8 |
| | Hz, 1H), 4.03 (td, J = 8.4, 4.9 Hz, 2H), 3.90 (q, J = 8.6 Hz, 1H), |
| | 2.29 - 2.22 (m, 2H), 2.17 (d, J = 8.7 Hz, 2H), 2.07 (s, 6H), 1.74 |
| | (dd, J = 15.1, 8.9 Hz, 1H), 0.94 (d, J = 6.7 Hz, 3H), 0.82 (d, J = |
| | 6.8 Hz, 3H), 0.56 (s, 9H). |
| 1545 | ¹H NMR (400 MHZ, DMSO-d6) & 12.99 (s, 1H), 8.62 (s, 1H), |
| | 7.93 (d, J = 7.3 Hz, 1H), 7.90 (s, 1H), 7.73 (s, 1H), 7.66 (s, 2H), |
| | 7.25 (t, J = 7.6 Hz, 1H), 7.12 (d, J = 7.8 Hz, 2H), 6.41 (s, 1H), |
| | 5.45 (d, J = 7.1 Hz, 1H), 4.69 (d, J = 15.8 Hz, 1H), 4.42 (d, J = |
| | 15.7 Hz, 1H), 4.34 (dt, J = 8.5, 5.8 Hz, 1H), 4.08 - 3.90 (m, |
| | 3H), 2.37 - 2.22 (m, 3H), 2.16 (ddd, J = 11.4, 8.8, 6.3 Hz, 1H), |
| | 2.11 - 1.85 (m, 6H), 1.73 (dd, J = 15.2, 9.0 Hz, 1H), 1.42 (d, J = |
| | 15.1 Hz, 1H), 0.96 (d, J = 6.8 Hz, 3H), 0.86 (d, J = 6.7 Hz, |
| | 3H), 0.57 (s, 9H). |
| 1546 | ¹H NMR (400 MHZ, Methanol-d4) 8 8.80 (d, J = 1.7 Hz, 1H), |
| | 8.51 (s, 2H), 8.02 (dt, J = 7.3, 1.7 Hz, 1H), 7.84 - 7.54 (m, 2H), |
| | 7.29 (t, J = 7.6 Hz, 1H), 7.16 (d, J = 7.6 Hz, 2H), 6.30 (s, 1H), |
| | 5.62 (dd, J = 10.9, 4.0 Hz, 1H), 5.09 (d, J = 16.5 Hz, 1H), 4.66 - |
| | 4.34 (m, 2H), 4.13 (t, J = 11.2 Hz, 1H), 3.99 (tt, J = 11.1, 3.7 |
| | Hz, 1H), 2.38 - 2.05 (m, 7H), 2.04 - 1.53 (m, 9H), 1.35 (d, J = |
| | 6.0 Hz, 3H), 1.16 - 1.05 (m, 1H), 1.01 (t, J = 7.4 Hz, 3H). |
| 1547 | ¹H NMR (400 MHZ, MEOD-d4) 88.81 (s, 1H), 8.54 (dd, J = |
| | 3.7, 2.1 Hz, 2H), 8.04 (ddd, J = 5.6, 3.6, 1.8 Hz, 1H), 7.82 - |
| | 7.54 (m, 2H), 7.30 (t, J = 7.6 Hz, 1H), 7.17 (d, J = 7.7 Hz, 2H), |
| | 6.30 (s, 1H), 5.65 (dd, J = 11.1, 4.0 Hz, 2H), 5.10 (d, J = 16.5 |
| | Hz, 1H), 4.58 (dt, J = 12.0, 5.6 Hz, 2H), 4.18 (t, J = 11.4 Hz, |
| | 1H), 4.00 (t, J = 11.0 Hz, 1H), 2.15 (s, 6H), 1.96 - 1.48 (m, 4H), |
| | 1.36 (d, J = 6.1 Hz, 3H), 1.32 - 1.10 (m, 2H), 1.03 (t, J = 7.5 |
| | Hz, 3H), 0.85 (s, 9H) |
| 1548 | ¹H NMR (400 MHZ, DMSO-d6) 8 8.61 (s, 1H), 8.18 (s, 2H), |
| | 7.95 - 7.83 (m, 1H), 7.63 (s, 2H), 7.21 (t, J = 7.6 Hz, 1H), 7.08 |
| | (d, J = 7.7 Hz, 2H), 6.35 (s, 1H), 5.30 (dd, J = 10.7, 4.2 Hz, |
| | 1H), 4.78 (d, J = 16.4 Hz, 1H), 4.41 (d, J = 16.4 Hz, 1H), 4.26 - |
| | 4.14 (m, 1H), 4.08 (t, J = 11.1 Hz, 1H), 3.75 (t, J = 13.2 Hz, |
| | 2H), 3.53 (t, J = 7.2 Hz, 2H), 3.12 (s, 1H), 2.56 - 2.48 (m, 2H), |
| | 2.12 - 1.80 (m, 6H), 1.55 (d, J = 14.9 Hz, 1H), 1.49 - 1.37 (m, |
| | 1H), 0.33 (s, 3H), 0.22 (p, J = 4.5 Hz, 1H), 0.16 - 0.06 (m, 1H), |
| | 0.04 - - 0.04 (m, 1H), -0.09 (p, J = 4.5 Hz, 1H). |
| 1552 | ¹H NMR (400 MHZ, DMSO-d6) 8 12.73 (s, 1H), 8.75 (s, 1H), |
| | 8.11 (d, J = 5.9 Hz, 1H), 7.93 (d, J = 7.3 Hz, 1H), 7.65 (s, 2H), |
| | 7.24 (d, J = 7.8 Hz, 1H), 7.11 (d, J = 7.7 Hz, 2H), 6.40 (s, 1H), |
| | 6.24 (d, J = 5.9 Hz, 1H), 5.51 (dd, J = 10.5, 4.0 Hz, 1H), 4.64 |
| | (d, J = 16.7 Hz, 1H), 4.43 (d, J = 15.7 Hz, 2H), 4.21 - 4.04 (m, |
| | 2H), 3.99 (s, 2H), 2.24 - 1.81 (m, 8H), 1.75 - 1.65 (m, 1H), 1.48 |
| | (d, J = 6.2 Hz, 3H), 1.39 (d, J = 14.8 Hz, 1H), 0.56 (s, 9H). |
| 1553 | ¹H NMR (400 MHz, DMSO-d6) 8 13.08 (s, 1H), 8.67 (s, 1H), |
| | 8.30 (d, J = 7.1 Hz, 1H), 7.98 (d, J = 7.2 Hz, 1H), 7.71 (d, J = |
| | 8.5 Hz, 2H), 7.27 (t, J = 7.6 Hz, 1H), 7.13 (d, J = 7.8 Hz, 2H), |
| | 6.82 (s, 1H), 6.46 (s, 1H), 5.46 (dd, J = 11.3, 4.2 Hz, 1H), 4.89 - |
| | 4.62 (m, 3H), 4.29 (m, 3H), 4.01 (m, 1H), 3.70 (m, 2H), 3.30 |
| | (s, 3H), 2.03 (d, J = 64.4 Hz, 7H), 1.63 (dd, J = 15.3, 9.0 Hz, |
| | 1H), 1.45 (d, J = 15.0 Hz, 1H), 1.24 (s, 1H), 0.57 (s, 9H). |
| 1554 | ¹H NMR (400 MHZ, Methanol-d4) 8 8.87 (s, 1H), 8.48 (s, 2H), |
| | 8.03 (d, J = 7.7 Hz, 1H), 7.85 - 7.52 (m, 2H), 7.27 (t, J = 7.6 |
| | Hz, 1H), 7.14 (d, J = 7.6 Hz, 2H), 6.26 (s, 1H), 5.62 (dd, J = |
| | 11.0, 4.4 Hz, 1H), 5.03 (d, J = 16.5 Hz, 1H), 4.61 - 4.43 (m, |
| | 2H), 4.37 (dddd, J = 12.0, 10.2, 4.5, 2.1 Hz, 1H), 4.11 (t, J = |
| | 11.4 Hz, 1H), 2.45 - 1.88 (m, 6H), 1.88 - 1.57 (m, 3H), 1.52 |
| | (dd, J = 15.2, 10.1 Hz, 1H), 1.33 (d, J = 6.1 Hz, 3H), 0.99 (t, J = |
| | 7.5 Hz, 3H), 0.52 (s, 3H), 0.38 (dt, J = 9.5, 4.9 Hz, 1H), 0.25 |
| | (dt, J = 9.6, 4.9 Hz, 1H), 0.13 (dt, J = 9.3, 5.0 Hz, 1H), 0.04 |
| | (dt, J = 9.7, 4.9 Hz, 1H). |
| 1558 | ¹H NMR (400 MHZ, DMSO-d6) 8 12.97 (s, 1H), 8.68 (s, 1H), |
| | 8.51 (s, 2H), 7.90 (s, 1H), 7.64 (s, 2H), 7.24 (d, J = 7.8 Hz, 1H), |
| | 7.11 (d, J = 7.8 Hz, 2H), 6.28 (s, 1H), 5.42 (d, J = 10.7 Hz, |
| | 1H), 4.95 (d, J = 16.4 Hz, 1H), 4.81 (hept, J = 6.0 Hz, 1H), |
| | 4.57 (d, J = 16.5 Hz, 1H), 4.57-4.48 (m, 1H), 4.14 - 4.01 (m, |
| | 1H), 2.07 (s, 6H), 1.30 (two overlapping d, J = 6.0 Hz, 6H), |
| | 1.21 - 1.07 (m, 1H), 0.93 (d, J = 6.8 Hz, 3H), 0.44 - 0.33 (m, |
| | 1H), 0.26 (d, J = 8.0 Hz, 2H), -0.05 - - 0.29 (m, 2H). |
| 1560 | ¹H NMR (400 MHZ, DMSO-d6) § 13.01 (s, 1H), 8.66 (s, 1H), |
| | 8.48 (s, 2H), 7.93 (s, 1H), 7.68 (s, 2H), 7.26 (t, J = 7.6 Hz, 1H), |
| | 7.13 (d, J = 7.6 Hz, 2H), 6.40 (s, 1H), 5.35 (dd, J = 10.6, 4.1 |
| | Hz, 1H), 4.81 (d, J = 16.4 Hz, 1H), 4.45 (d, J = 16.4 Hz, 1H), |
| | 4.30 - 4.18 (m, 1H), 4.13 (t, J = 11.1 Hz, 1H), 3.29 - 3.22 (m, |
| | 4H), 1.99 (s, 6H), 1.60 (d, J = 14.8 Hz, 1H), 1.53 - 1.46 (m, |
| | 1H), 1.46 - 1.40 (m, 4H), 0.96 (s, 6H), 0.37 (s, 3H), 0.27 (dt, J = |
| | 9.5, 4.5 Hz, 1H), 0.16 (dt, J = 9.5, 4.7 Hz, 1H), 0.05 (dt, J = |
| | 9.1, 4.8 Hz, 1H), -0.04 (dt, J = 5.5, 3.0 Hz, 1H). |
| 1561 | ¹H NMR (400 MHZ, DMSO-d6) 8 8.64 (d, J = 1.7 Hz, 1H), 8.13 |
| | (s, 2H), 7.93 (dt, J = 6.4, 2.1 Hz, 1H), 7.73 - 7.61 (m, 2H), 7.26 |
| | (t, J = 7.6 Hz, 1H), 7.13 (d, J = 7.7 Hz, 2H), 6.41 (s, 1H), 5.35 |
| | (dd, J = 10.5, 4.0 Hz, 1H), 4.83 (d, J = 16.2 Hz, 1H), 4.44 (d, J = |
| | 16.2 Hz, 1H), 4.27 - 4.18 (m, 1H), 4.13 (t, J = 11.1 Hz, 1H), |
| | 3.40 (t, J = 7.0 Hz, 2H), 3.09 (s, 2H), 2.02 (s, 6H), 1.78 (t, J = |
| | 7.0 Hz, 2H), 1.61 (dd, J = 15.3, 2.2 Hz, 1H), 1.48 (dd, J = 15.1, |
| | 9.6 Hz, 1H), 1.10 (s, 6H), 0.38 (s, 3H), 0.27 (dt, J = 9.5, 4.6 Hz, |
| | 1H), 0.16 (dt, J = 9.4, 4.7 Hz, 1H), 0.05 (t, J = 9.1, 4.45 Hz, |
| | 1H), -0.04 (dt, J = 9.6, 4.6 Hz, 1H). (missing N-H) |
| 1562 | ¹H NMR (400 MHZ, DMSO-d6) 8 13.06 (s, 1H), 8.66 (s, 1H), |
| | 8.54 (s, 2H), 7.93 (s, 1H), 7.68 (s, 2H), 7.26 (t, J = 7.6 Hz, 1H), |
| | 7.13 (d, J = 7.6 Hz, 2H), 6.41 (s, 1H), 5.36 (dd, J = 10.4, 3.8 |
| | Hz, 1H), 4.84 (d, J = 16.5 Hz, 1H), 4.53 (d, J = 16.6 Hz, 1H), |
| | 4.31 - 4.21 (m, 1H), 4.21 - 4.13 (m, 1H), 4.11 (t, J = 6.5 Hz, |
| | 2H), 1.99 (s, 6H), 1.75 (h, J = 7.1 Hz, 2H), 1.61 (d, J = 14.8 |
| | Hz, 1H), 1.48 (dd, J = 15.1, 9.5 Hz, 1H), 0.98 (t, J = 7.4 Hz, |
| | 3H), 0.37 (s, 3H), 0.27 (dt, J = 9.4, 4.8 Hz, 1H), 0.16 (dt, J = |
| | 9.3, 4.7 Hz, 1H), 0.05 (dt, J = 9.2, 4.8 Hz, 1H), -0.04 (dt, J = |
| | 9.6, 4.6 Hz, 1H). |
| 1563 | ¹H NMR (400 MHZ, DMSO) 8 13.01 (s, 1H), 8.62 (s, 1H), 8.03 |
| | (s, 2H), 7.91 (s, 1H), 7.64 (s, 2H), 7.25 (s, 1H), 7.12 (s, 2H), |
| | 6.36 (s, 1H), 5.34 (s, 1H), 4.87 (d, J = 16.3 Hz, 1H), 4.51 (d, J = |
| | 16.4 Hz, 1H), 4.10 (s, 1H), 4.01 (d, J = 12.0 Hz, 1H), 3.64 |
| | (s, 4H), 1.98 (s, 5H), 1.75 (t, J = 12.1 Hz, 1H), 1.29 (s, 6H), |
| | 1.25 - 1.12 (m, 3H), 0.75 (d, J = 6.6 Hz, 3H), 0.28 (d, J = 6.3 |
| | Hz, 3H). |
| 1564 | ¹H NMR (400 MHZ, DMSO-d6) 8 13.01 (br. s., 1H), 8.66 (s, |
| | 1H), 8.07 (s, 2H), 7.92 (d, J = 6.4 Hz, 1H), 7.72 - 7.54 (m, 2H), |
| | 7.26 (t, J = 6.8 Hz, 1H), 7.12 (d, J = 7.1 Hz, 2H), 6.35 (br. s., |
| | 1H), 5.37 (d, J = 7.3 Hz, 1H), 4.87 (d, J = 16.1 Hz, 1H), 4.52 |
| | (d, J = 16.4 Hz, 1H), 4.25 - 4.07 (m, 2H), 3.38 (t, J = 6.8 Hz, |
| | 2H), 3.08 (s, 2H), 2.29 - 2.18 (m, 1H), 1.97 (br. s., 6H), 1.77 (t, |
| | J = 7.0 Hz, 3H), 1.16 - 1.08 (m, 9H), 0.90 (d, J = 21.8 Hz, 3H). |
| 1569 | ¹H NMR (400 MHZ, DMSO-d6) 8 8.49 (s, 1H), 7.82 (d, J = 7.9 |
| | Hz, 1H), 7.69 - 7.47 (m, 3H), 7.11 (t, J = 7.6 Hz, 1H), 6.98 (d, |
| | J = 7.7 Hz, 2H), 6.61 (s, 1H), 6.29 (s, 1H), 5.22 - 5.07 (m, 1H), |
| | 4.79 - 4.59 (m, 1H), 4.26 - 4.06 (m, 3H), 3.62 - 3.49 (m, 3H), |
| | 2.03 - 1.74 (m, 9H), 1.60 (s, 1H), 1.48 (d, J = 15.0 Hz, 1H), |
| | 1.29 (dd, J = 15.7, 8.5 Hz, 1H), 1.06 (d, J = 6.2 Hz, 4H), 0.15 |
| | (s, 3H), 0.09 - - 0.03 (m, 2H), -0.05 - - 0.14 (m, 1H), -0.18 - - |
| | 0.31 (m, 1H). |
| 1574 | ¹H NMR (400 MHZ, DMSO-d6) 8 14.25 (s, 1H), 8.71 (t, J = 1.8 |
| | Hz, 1H), 8.51 (very broad s, 3H), 8.18 (d, J = 7.9 Hz, 1H), 7.81 |
| | (t, J = 7.7 Hz, 1H), 7.74 (d, J = 7.9 Hz, 1H), 7.25 (t, J = 7.9 |
| | Hz, 1H), 7.19 - 7.03 (m, 2H), 6.56 (s, 1H), 5.38 (dd, J = 10.9, |
| | 4.3 Hz, 1H), 4.86 (d, J = 12 Hz, 1H), 4.81 (hept, J = 6.2 Hz, |
| | 1H), 4.67 (d, J = 16.6 Hz, 1H), 4.17 (s, 1H), 3.83 - 3.70 (m, |
| | 1H), 1.97 (s, 6H), 1.79 (dd, J = 15.2, 8.9 Hz, 1H), 1.42 (d, J = |
| | 15.0 Hz, 1H), 1.30 (two overlapping d, J = 6.0 Hz, 6H), 0.58 |
| | (s, 9H). |
| 1575 | ¹H NMR (400 MHZ, DMSO-d6) 8 14.22 (s, 1H), 8.87 (s, 2H), |
| | 8.51 (very broad s, 2H), 8.11 (s, 1H), 7.79 (d, J = 4.8 Hz, 2H), |
| | 7.29 (t, J = 7.6 Hz, 1H), 7.15 (d, J = 7.6 Hz, 2H), 6.63 (s, 1H), |
| | 5.51 (dd, J = 11.0, 4.2 Hz, 1H), 4.91 (d, J = 16.6 Hz, 1H), 4.78 |
| | (hept, J = 6.0 Hz, 1H), 4.67 (d, J = 16.7 Hz, 1H), 4.22 (s, 1H), |
| | 4.04 - 3.82 (m, 1H), 2.01 (s, 6H), 1.81 (dd, J = 15.2, 8.7 Hz, |
| | 1H), 1.43 (d, J = 14.7 Hz, 1H), 1.31 (d, J = 6.0 Hz, 6H), 0.59 |
| | (s, 9H). |
| 1576 | ¹H NMR (400 MHZ, DMSO-d6) & 13.07 (s, 1H), 8.65 (s, 1H), |
| | 8.45 (s, 2H), 7.94 (s, 1H), 7.67 (s, 2H), 7.26 (t, J = 7.7 Hz, 1H), |
| | 7.13 (d, J = 7.7 Hz, 2H), 6.41 (s, 1H), 5.34 (d, J = 7.7 Hz, 1H), |
| | 4.89 (p, J = 7.1 Hz, 1H), 4.82 (d, J = 16.7 Hz, 1H), 4.53 (d, J = |
| | 16.5 Hz, 1H), 4.26 (q, J = 11.4 Hz, 1H), 4.18 (q, J = 10.9 Hz, |
| | 1H), 2.49 - 2.39 (m, 2H), 2.23 - 2.01 (m, 2H), 1.96 (s, 6H), 1.85 - |
| | 1.75 (m, 1H), 1.70 - 1.54 (m, 2H), 1.47 (dd, J = 15.2, 9.4 Hz, |
| | 1H), 0.36 (s, 3H), 0.26 (dt, J = 9.5, 4.6 Hz, 1H), 0.15 (dt, J = |
| | 9.5, 4.7 Hz, 1H), 0.05 (dt, J = 9.2, 4.4 Hz, 1H), -0.01 - - 0.10 |
| | (m, 1H). |
| 1577 | ¹H NMR (400 MHZ, DMSO-d6) 8 13.02 (s, 1H), 8.62 (s, 1H), |
| | 8.55 (s, 2H), 7.92 (d, J = 7.0 Hz, 1H), 7.65 (s, 2H), 7.26 (t, J = |
| | 7.6 Hz, 1H), 7.13 (d, J = 7.6 Hz, 2H), 6.39 (s, 1H), 5.36 (dd, J = |
| | 11.0, 4.1 Hz, 1H), 4.91 (d, J = 16.5 Hz, 1H), 4.59 (d, J = 16.5 |
| | Hz, 1H), 4.17 (t, J = 11.2 Hz, 1H), 4.11 (t, J = 6.5 Hz, 2H), |
| | 4.02 - 3.86 (m, 1H), 2.04 (s, 6H), 1.82 - 1.69 (m, 3H), 1.58 - |
| | 1.45 (m, 1H), 1.12 (p, J = 6.5 Hz, 1H), 1.08 - 1.01 (m, 1H), |
| | 0.98 (t, J = 7.4 Hz, 3H), 0.91 - 0.79 (m, 1H), 0.72 (d, J = 6.5 |
| | Hz, 3H), 0.65 (d, J = 6.4 Hz, 3H). |
| 1310 | ¹H NMR (400 MHZ, DMSO-d6) 8 12.91 (br. s, 1H), 8.68 (s, |
| | 1H), 8.03 - 7.82 (m, 3H), 7.68 (br. s, 2H), 7.32 - 7.20 (m, 1H), |
| | 7.12 (d, J = 7.3 Hz, 2H), 6.44 (br. s, 1H), 5.40 (dd, J = 10.8, |
| | 4.2 Hz, 1H), 4.89 - 4.66 (m, 2H), 4.45 (d, J = 15.7 Hz, 1H), |
| | 4.31 (t, J = 11.0 Hz, 1H), 4.05 (br. s., 1H), 3.06 (s, 3H), 2.24 - |
| | 1.84 (m, 10H), 1.76 (dd, J = 15.4, 9.0 Hz, 1H), 1.70 - 1.58 (m, |
| | 2H), 1.42 (d, J = 14.9 Hz, 1H), 0.56 (s, 9H). , ¹H NMR (400 |
| | MHz, Chloroform-d) § 8.86 (t, J = 1.8 Hz, 1H), 8.11 - 8.07 (m, |
| | 1H), 7.92 - 7.80 (m, 3H), 7.67 (t, J = 7.7 Hz, 1H), 7.22 (t, J = |
| | 7.6 Hz, 1H), 7.08 (d, J = 7.6 Hz, 2H), 6.28 (s, 1H), 5.47 (dd, J = |
| | 11.1, 4.3 Hz, 1H), 5.08 (d, J = 15.6 Hz, 1H), 4.57 - 4.46 (m, |
| | 1H), 4.31 - 4.22 (m, 1H), 4.16 (d, J = 15.8 Hz, 1H), 4.06 (t, J = |
| | 11.4 Hz, 1H), 3.19 (s, 3H), 2.35 - 2.27 (m, 2H), 2.26 - 2.19 (m, |
| | 2H), 2.03 (s, 6H), 1.76 (dt, J = 10.9, 3.9 Hz, 2H), 1.70 - 1.66 |
| | (m, 1H), 1.56 (d, J = 14.6 Hz, 1H), 0.62 (s, 9H). Sulfonamide - |
| | NH not visible |
| 1578 | ¹H NMR (400 MHZ, Chloroform-d) 8 14.49 (s, 1H), 8.79 (s, |
| | 1H), 8.13 (d, J = 7.8 Hz, 1H), 7.89 (dt, J = 7.7, 1.4 Hz, 1H), |
| | 7.78 - 7.64 (m, 2H), 7.22 (t, J = 7.6 Hz, 1H), 7.07 (d, J = 7.6 |
| | Hz, 2H), 6.90 (s, 1H), 6.81 (s, 1H), 6.24 (s, 1H), 5.72 - 5.46 (m, |
| | 1H), 5.34 (d, J = 16.1 Hz, 1H), 5.19 (s, 1H), 4.68 (s, 1H), 4.27 |
| | (d, J = 18.0 Hz, 2H), 3.38 (s, 3H), 2.44 (d, J = 33.6 Hz, 2H), |
| | 2.32 - 2.20 (m, 2H), 2.01 (s, 6H), 1.82 (s, 2H), 1.59 (s, 2H), 0.60 |
| | (s, 9H). |
| 1579 | ¹H NMR (400 MHZ, Chloroform-d) 8 8.84 (d, J = 2.4 Hz, 1H), |
| | 8.08 (d, J = 7.9 Hz, 1H), 7.92 - 7.84 (m, 2H), 7.78 (d, J = 2.3 |
| | Hz, 1H), 7.66 (td, J = 7.8, 2.3 Hz, 1H), 7.22 (td, J = 8.0, 2.4 |
| | Hz, 1H), 7.08 (d, J = 7.6 Hz, 2H), 6.32 - 6.25 (m, 1H), 5.44 (dd, |
| | J = 10.6, 3.9 Hz, 1H), 5.05 (dd, J = 15.6, 2.3 Hz, 1H), 4.46 - |
| | 4.26 (m, 2H), 4.16 (d, J = 15.6 Hz, 1H), 4.06 (t, J = 11.4 Hz, |
| | 1H), 3.84 - 3.67 (m, 2H), 2.40 - 2.32 (m, 2H), 2.18 (q, J = 10.1 |
| | Hz, 2H), 2.03 (s, 6H), 1.82 - 1.75 (m, 2H), 1.67 (dd, J = 8.9, |
| | 6.8 Hz, 1H), 1.55 (d, J = 15.0 Hz, 1H), 1.19 (td, J = 7.1, 2.3 |
| | Hz, 3H), 0.60 (s, 9H). Sulfonamide NH not visisble. |
| 1580 | ¹H NMR (400 MHZ, Chloroform-d) 8 8.81 (s, 1H), 8.09 (d, J = |
| | 7.8 Hz, 1H), 7.96 - 7.82 (m, 3H), 7.68 (t, J = 7.8 Hz, 1H), 7.23 |
| | (t, J = 7.6 Hz, 1H), 7.09 (d, J = 7.6 Hz, 2H), 6.31 (s, 1H), 5.45 |
| | (d, J = 9.0 Hz, 1H), 5.03 (d, J = 13.6 Hz, 1H), 4.36 - 4.29 (m, |
| | 1H), 4.27 - 4.20 (m, 1H), 4.12 - 4.04 (m, 1H), 3.81 - 3.71 (m, |
| | 3H), 3.70 - 3.59 (m, 3H), 3.27 (s, 3H), 2.05 (s, 6H), 1.70 - 1.62 |
| | (m, 1H), 1.60 - 1.53 (m, 1H), 1.24 (t, J = 6.8 Hz, 3H), 0.61 (s, |
| | 9H). Sulfonamide NH not visible |
| 1582 | ¹H NMR (400 MHZ, DMSO-d6) 8 13.10 (s, 1H), 8.90 (s, 1H), |
| | 8.68 (s, 1H), 8.46 (s, 1H), 7.95 (s, 1H), 7.68 (s, 2H), 7.26 (t, J = |
| | 7.6 Hz, 1H), 7.13 (d, J = 7.7 Hz, 2H), 6.43 (s, 1H), 5.39 (dd, |
| | J = 11.0, 4.3 Hz, 1H), 4.92 (d, J = 16.3 Hz, 1H), 4.66 (d, J = |
| | 16.4 Hz, 1H), 4.31 (t, J = 11.2 Hz, 1H), 4.05 (q, J = 9.1, 6.6 |
| | Hz, 1H), 3.77 (s, 3H), 3.44 (s, 3H), 2.03 (d, J = 70.8 Hz, 6H), |
| | 1.79 - 1.72 (m, 1H), 1.45 (d, J = 15.0 Hz, 1H), 0.58 (s, 9H). |
| 1583 | ¹H NMR (400 MHZ, DMSO-d6) 8 13.07 (s, 1H), 8.66 (s, 1H), |
| | 7.95 (s, 1H), 7.84 (t, J = 7.8 Hz, 1H), 7.68 (s, 2H), 7.41 (d, J = |
| | 8.0 Hz, 1H), 7.33 (d, J = 7.6 Hz, 1H), 7.26 (t, J = 7.6 Hz, 1H), |
| | 7.12 (d, J = 7.7 Hz, 2H), 6.42 (s, 1H), 5.37 (dd, J = 10.8, 4.2 |
| | Hz, 1H), 4.84 (d, J = 16.0 Hz, 1H), 4.60 (d, J = 16.0 Hz, 1H), |
| | 4.26 (t, J = 11.2 Hz, 1H), 4.09 - 3.96 (m, 1H), 3.35 (s, 3H), 2.22 - |
| | 1.87 (m, 9H), 1.77 (dd, J = 15.2, 8.7 Hz, 1H), 1.42 (d, J = |
| | 15.0 Hz, 1H), 0.56 (s, 9H). |
| 1584 | ¹H NMR (400 MHZ, DMSO-d6) 8 13.07 (s, 1H), 8.71 (s, 1H), |
| | 7.95 (d, J = 6.4 Hz, 1H), 7.77 (t, J = 7.9 Hz, 1H), 7.68 (d, J = |
| | 5.5 Hz, 2H), 7.56 (d, J = 8.2 Hz, 1H), 7.26 (t, J = 7.6 Hz, 1H), |
| | 7.21 (d, J = 7.5 Hz, 1H), 7.13 (d, J = 7.7 Hz, 2H), 6.48 - 6.37 |
| | (m, 1H), 5.40 (dd, J = 10.7, 4.3 Hz, 1H), 4.85 (d, J = 16.1 Hz, |
| | 1H), 4.57 (d, J = 16.1 Hz, 1H), 4.25 (t, J = 11.1 Hz, 1H), 4.08 - |
| | 4.00 (m, 1H), 3.72 (s, 3H), 3.43 (s, 3H), 2.02 (d, J = 49.8 Hz, |
| | 6H), 1.76 (dd, J = 15.2, 8.9 Hz, 1H), 1.42 (d, J = 14.9 Hz, 1H), |
| | 0.57 (s, 9H). |
| 1311 | ¹H NMR (400 MHZ, DMSO-d6) 8 13.02 (s, 1H), 8.62 (s, 1H), |
| | 8.50 (s, 2H), 7.95 - 7.88 (m, 1H), 7.65 (d, J = 4.9 Hz, 2H), 7.26 |
| | (t, J = 7.6 Hz, 1H), 7.13 (d, J = 7.6 Hz, 2H), 6.38 (s, 1H), 5.35 |
| | (dd, J = 10.6, 3.9 Hz, 1H), 4.90 (d, J = 16.3 Hz, 1H), 4.51 (d, J = |
| | 16.4 Hz, 1H), 4.14 (t, J = 11.1 Hz, 1H), 4.04 (td, J = 11.1, |
| | 10.2, 5.3 Hz, 1H), 3.28 - 3.25 (m, 3H), 2.05 (s, 6H), 1.83 (ddt, |
| | J = 13.7, 9.0, 4.4 Hz, 1H), 1.64 (dtd, J = 10.6, 7.4, 3.8 Hz, 1H), |
| | 1.47 - 1.40 (m, 4H), 0.96 (s, 9H), 0.35 - 0.27 (m, 1H), 0.18 - |
| | 0.07 (m, 2H), -0.20 - - 0.29 (m, J = 3.9 Hz, 2H). |
| 1585 | ¹H NMR (400 MHZ, DMSO-d6) § 12.96 (br. s., 1H), 8.66 (br. |
| | s., 1H), 7.96 (br. s., 1H), 7.70 (br. s., 2H), 7.45 (t, J = 7.9 Hz, |
| | 1H), 7.26 (t, J = 4.8 Hz, 1H), 7.12 (d, J = 7.1 Hz, 2H), 6.58 (d, |
| | J = 7.3 Hz, 1H), 6.42 (br. s., 1H), 6.38 (d, J = 8.6 Hz, 1H), 5.23 |
| | (d, J = 6.8 Hz, 1H), 5.16 (t, J = 5.1 Hz, 1H), 4.75 (d, J = 15.4 |
| | Hz, 1H), 4.34 (d, J = 15.7 Hz, 1H), 4.15 - 4.01 (m, 2H), 3.85 - |
| | 3.74 (m, 1H), 3.70 - 3.63 (m, 1H), 3.50 - 3.40 (m, 2H), 2.19 - |
| | 2.10 (m, 2H), 2.06 - 1.76 (m, 8H), 1.72 - 1.61 (m, 1H), 1.45 (s, |
| | 3H), 1.37 (d, J = 14.7 Hz, 1H), 0.53 (s, 9H). |
| 1586 | ¹H NMR (400 MHZ, DMSO-d6) 8 12.98 (br. s., 1H), 8.71 (br. |
| | s., 1H), 7.95 (br. s., 1H), 7.69 (br. s., 2H), 7.45 (t, J = 7.8 Hz, |
| | 1H), 7.26 (t, J = 4.8 Hz, 1H), 7.12 (d, J = 7.1 Hz, 2H), 6.58 (d, |
| | J = 7.3 Hz, 1H), 6.42 (br. s., 1H), 6.37 (d, J = 8.3 Hz, 1H), 5.44 - |
| | 5.37 (m, 1H), 5.35 - 5.26 (m, 1H), 4.70 (d, J = 15.9 Hz, 1H), |
| | 4.34 (d, J = 15.4 Hz, 1H), 4.19 - 4.02 (m, 2H), 3.89 - 3.79 (m, |
| | 1H), 3.74 - 3.67 (m, 1H), 3.49 - 3.38 (m, 2H), 2.15 - 2.08 (m, |
| | 2H), 2.03 - 1.75 (m, 8H), 1.71 - 1.63 (m, 1H), 1.39 (s, 3H), 1.38 - |
| | 1.34 (m, 1H), 0.53 (s, 9H). |
| 1588 | ¹H NMR (400 MHZ, THF-d8) 8 8.94 (s, 1H), 8.07 (s, 1H), 7.83 |
| | (t, J = 7.7 Hz, 1H), 7.63 (s, 2H), 7.48 (d, J = 7.7 Hz, 1H), 7.40 |
| | (d, J = 7.7 Hz, 1H), 7.10 (d, J = 40.5 Hz, 4H), 5.03 (d, J = 15.9 |
| | Hz, 1H), 4.28 (s, 1H), 4.05 (s, 3H), 3.02 (s, 2H), 1.83 (s, 4H), |
| | 1.62 (d, J = 6.7 Hz, 8H), 0.94 - 0.85 (m, 7H), 0.66 (s, 9H). |
| 1589 | 'H NMR (400 MHZ, THF-d8) 8 8.96 (s, 1H), 8.08 (s, 1H), 7.83 |
| | (t, J = 7.7 Hz, 1H), 7.65 (s, 2H), 7.47 (d, J = 7.8 Hz, 1H), 7.38 |
| | (d, J = 7.7 Hz, 1H), 7.18 - 6.99 (m, 4H), 4.40 - 4.27 (m, 1H), |
| | 4.27 - 4.16 (m, 1H), 3.17 - 3.00 (m, 2H), 2.96 - 2.84 (m, 2H), |
| | 1.86 - 1.74 (m, 6H), 1.66 - 1.53 (m, 7H), 0.93 - 0.83 (m, 7H), |
| | 0.65 (s, 9H). |
| 1590 | 'H NMR (400 MHZ, THF-d8) 8 9.04 (s, 1H), 8.06 (s, 1H), 7.76 - |
| | 7.62 (m, 3H), 7.33 (d, J = 7.7 Hz, 1H), 7.22 (d, J = 7.5 Hz, |
| | 2H), 7.11 (d, J = 7.4 Hz, 2H), 6.24 (s, 1H), 5.16 - 4.98 (m, 3H), |
| | 4.54 (d, J = 16.2 Hz, 1H), 4.30 - 4.19 (m, 1H), 4.10 (t, J = 11.0 |
| | Hz, 1H), 2.33 (p, J = 7.0 Hz, 2H), 2.21 - 1.92 (m, 6H), 1.64 - |
| | 1.52 (m, 5H), 1.02 (d, J = 6.7 Hz, 4H), 0.81 (d, J = 6.8 Hz, 3H), |
| | 0.69 (s, 9H). |
| 1591 | ¹H NMR (400 MHZ, THF-d8) 8 9.17 (s, 1H), 7.79 - 7.62 (m, |
| | 3H), 7.30 (d, J = 7.8 Hz, 1H), 7.23 (d, J = 7.6 Hz, 2H), 7.10 (s, |
| | 2H), 6.23 (s, 1H), 4.56 (d, J = 16.7 Hz, 2H), 4.21 (s, 1H), 3.87 |
| | (s, 1H), 2.67 - 2.46 (m, 2H), 1.80 (dd, J = 15.2, 7.9 Hz, 2H), |
| | 1.68 - 1.54 (m, 3H), 1.47 - 1.37 (m, 7H), 1.07 (d, J = 5.0 Hz, |
| | 3H), 0.93 - 0.78 (m, 6H), 0.70 (s, 9H). |
| 1592 | ¹H NMR (400 MHZ, Methanol-d4) 8 9.07 (s, 1H), 8.06 (d, J = |
| | 7.4 Hz, 1H), 7.77 - 7.63 (m, 3H), 7.34 - 7.21 (m, 3H), 7.13 (d, |
| | J = 7.6 Hz, 2H), 6.25 (s, 1H), 5.81 (dd, J = 11.0, 4.0 Hz, 1H), |
| | 5.11 (d, J = 16.5 Hz, 1H), 4.55 (d, J = 16.5 Hz, 2H), 4.17 (dd, |
| | J = 7.7, 4.4 Hz, 1H), 4.01 (t, J = 11.2 Hz, 1H), 3.68 - 3.61 (m, |
| | 1H), 1.89 (s, 3H), 1.46 - 1.34 (m, 8H), 0.92 - 0.82 (m, 4H), 0.70 |
| | (s, 9H). |
| 1593 | ¹H NMR (400 MHZ, Methanol-d4) 8 8.99 (s, 1H), 8.05 (d, J = |
| | 7.6 Hz, 1H), 7.77 - 7.66 (m, 3H), 7.33 (d, J = 7.7 Hz, 1H), 7.24 |
| | (dd, J = 9.9, 7.6 Hz, 2H), 7.13 (d, J = 7.6 Hz, 2H), 6.26 (s, 1H), |
| | 5.77 - 5.70 (m, 1H), 5.13 - 4.99 (m, 2H), 4.54 (d, J = 16.2 Hz, |
| | 2H), 4.16 (q, J = 11.6, 9.7 Hz, 2H), 3.67 - 3.60 (m, 1H), 1.85 |
| | (dd, J = 15.3, 8.0 Hz, 1H), 1.81 (s, 3H), 1.55 (d, J = 7.0 Hz, |
| | 3H), 1.40 - 1.34 (m, 3H), 0.95 - 0.82 (m, 4H), 0.69 (s, 9H). |
| 1298 | ¹H NMR (400 MHZ, DMSO-d6) 8 8.54 (s, 3H), 7.66 (s, 1H), |
| | 7.42 (d, J = 32.4 Hz, 2H), 7.23 (s, 1H), 7.14 - 7.04 (m, 2H), |
| | 6.27 (s, 1H), 5.19 (d, J = 9.7 Hz, 1H), 4.79 (p, J = 6.0 Hz, 1H), |
| | 4.18 - 4.04 (m, 2H), 3.99 (d, J = 14.1 Hz, 1H), 3.84 (dd, J = |
| | 14.6, 6.8 Hz, 2H), 2.81 - 2.69 (m, 1H), 2.21 - 1.85 (m, 6H), 1.80 |
| | (dd, J = 14.7, 4.1 Hz, 1H), 1.30 (d, J = 6.1 Hz, 6H), 0.91 - 0.79 |
| | (m, 2H), 0.57 (s, 9H). |
| 1594 | ¹H NMR (400 MHZ, DMSO-d6) 8 12.98 (s, 1H), 8.57 (s, 1H), |
| | 8.03 (s, 2H), 7.89 (s, 1H), 7.65 (s, 2H), 7.26 (t, J = 7.5 Hz, 1H), |
| | 7.13 (d, J = 7.6 Hz, 2H), 6.38 (s, 1H), 5.32 (dd, J = 11.0, 4.2 |
| | Hz, 1H), 4.87 (d, J = 16.3 Hz, 1H), 4.47 (d, J = 16.3 Hz, 1H), |
| | 4.09 (t, J = 10.2 Hz, 1H), 3.85 (d, J = 11.3 Hz, 1H), 3.64 (s, |
| | 4H), 2.04 (q, J = 21.2, 14.5 Hz, 6H), 1.85 (td, J = 12.9, 10.8, |
| | 5.6 Hz, 2H), 1.76 - 1.63 (m, 1H), 1.60 - 1.42 (m, 4H), 1.29 (s, |
| | 7H), 0.93 - 0.82 (m, 1H). |
| 1595 | ¹H NMR (400 MHZ, DMSO-d6) 8 12.96 (s, 1H), 8.61 (s, 1H), |
| | 8.03 (s, 2H), 7.90 (d, J = 5.7 Hz, 1H), 7.71 - 7.55 (m, 2H), 7.26 |
| | (t, J = 7.6 Hz, 1H), 7.13 (d, J = 7.6 Hz, 2H), 6.37 (s, 1H), 5.35 |
| | (dd, J = 10.5, 3.7 Hz, 1H), 4.90 (d, J = 16.3 Hz, 1H), 4.48 (d, J = |
| | 16.4 Hz, 1H), 4.10 (t, J = 11.0 Hz, 1H), 4.06 - 3.95 (m, 1H), |
| | 3.64 (s, 4H), 2.04 (s, 6H), 1.87 - 1.78 (m, 1H), 1.63 (td, J = 7.2, |
| | 3.4 Hz, 1H), 1.29 (s, 6H), 1.03 - 0.90 (m, 2H), 0.31 (s, 1H), 0.17 - |
| | 0.05 (m, 2H), -0.24 (q, J = 4.1, 3.1 Hz, 2H). |
| 1312 | ¹H NMR (400 MHZ, DMSO-d6) 8 8.70 (d, J = 1.9 Hz, 1H), 8.09 |
| | (d, J = 8.2 Hz, 1H), 8.03 (s, 2H), 7.94 (s, 2H), 7.71 (t, J = 7.7 |
| | Hz, 1H), 7.61 (d, J = 7.6 Hz, 1H), 7.14 (t, J = 7.5 Hz, 1H), 7.04 |
| | (d, J = 7.6 Hz, 2H), 6.15 (s, 1H), 5.31 (dd, J = 10.1, 3.4 Hz, |
| | 1H), 4.83 (d, J = 16.4 Hz, 1H), 4.52 (d, J = 16.4 Hz, 1H), 3.92 - |
| | 3.73 (m, 2H), 3.64 (s, 4H), 1.92 (s, 6H), 1.74 (dd, J = 15.1, |
| | 8.5 Hz, 1H), 1.42 (d, J = 14.9 Hz, 1H), 1.29 (s, 6H), 0.55 (s, |
| | 9H). |
| 1596 | ¹H NMR (400 MHZ, DMSO-d6) 8 13.02 (s, 1H), 8.63 (s, 1H), |
| | 8.54 (s, 2H), 7.91 (s, 1H), 7.64 (s, 2H), 7.26 (t, J = 7.6 Hz, 1H), |
| | 7.13 (d, J = 7.6 Hz, 2H), 6.37 (s, 1H), 5.37 (d, J = 7.2 Hz, 1H), |
| | 4.92 (d, J = 16.5 Hz, 1H), 4.59 (d, J = 16.5 Hz, 1H), 4.21 (q, J = |
| | 6.9 Hz, 3H), 4.10 - 4.01 (m, 1H), 2.03 (s, 6H), 1.82 (p, J = |
| | 8.3, 7.2 Hz, 1H), 1.64 (dt, J = 10.7, 3.5 Hz, 1H), 1.35 (t, J = 7.0 |
| | Hz, 3H), 0.98 (ddp, J = 21.2, 14.1, 7.4 Hz, 2H), 0.31 (s, 1H), |
| | 0.12 (d, J = 10.6 Hz, 2H), -0.25 (s, 2H). |
| 1597 | ¹H NMR (400 MHZ, DMSO-d6) & 13.02 (s, 1H), 8.62 (s, 1H), |
| | 8.52 (s, 2H), 7.91 (s, 1H), 7.64 (s, 2H), 7.26 (t, J = 7.6 Hz, 1H), |
| | 7.13 (d, J = 7.6 Hz, 2H), 6.37 (s, 1H), 5.36 (d, J = 9.4 Hz, 1H), |
| | 4.91 (d, J = 16.5 Hz, 1H), 4.81 (hept, J = 6.0 Hz, 1H), 4.58 (d, |
| | J = 16.5 Hz, 1H), 4.19 (t, J = 11.1 Hz, 1H), 4.07 (d, J = 10.4 |
| | Hz, 1H), 2.03 (s, 6H), 1.82 (q, J = 7.5, 6.9 Hz, 1H), 1.63 (dt, J = |
| | 10.7, 3.6 Hz, 1H), 1.30 (dd, J = 6.0, 2.2 Hz, 6H), 0.97 (tt, J = |
| | 14.0, 7.1 Hz, 2H), 0.31 (s, 1H), 0.12 (d, J = 10.5 Hz, 2H), - |
| | 0.25 (s, 2H). |
| 1598 | ¹H NMR (400 MHZ, DMSO-d6) 8 13.02 (s, 1H), 8.63 (s, 1H), |
| | 8.53 (s, 2H), 7.91 (s, 1H), 7.64 (s, 2H), 7.25 (d, J = 7.6 Hz, 1H), |
| | 7.13 (d, J = 7.7 Hz, 2H), 6.37 (s, 1H), 5.37 (d, J = 10.0 Hz, |
| | 1H), 4.91 (d, J = 16.5 Hz, 1H), 4.59 (dt, J = 11.9, 6.1 Hz, 2H), |
| | 4.19 (t, J = 11.1 Hz, 1H), 4.06 (d, J = 5.8 Hz, 1H), 2.06 (d, J = |
| | 29.5 Hz, 6H), 1.84 (dd, J = 15.3, 8.8 Hz, 1H), 1.74 - 1.57 (m, |
| | 3H), 1.26 (d, J = 6.0 Hz, 3H), 0.93 (t, J = 7.4 Hz, 5H), 0.30 (s, |
| | 1H), 0.12 (d, J = 11.5 Hz, 2H), -0.25 (s, 2H). |
| 1599 | ¹H NMR (400 MHZ, DMSO-d6) 8 13.03 (s, 1H), 8.62 (s, 1H), |
| | 8.54 (s, 2H), 7.92 (s, 1H), 7.65 (s, 2H), 7.25 (d, J = 7.5 Hz, 1H), |
| | 7.12 (d, J = 7.6 Hz, 2H), 6.38 (s, 1H), 5.36 (d, J = 10.2 Hz, |
| | 1H), 4.91 (d, J = 16.5 Hz, 1H), 4.59 (d, J = 16.5 Hz, 1H), 4.26 - |
| | 4.11 (m, 3H), 3.95 (s, 1H), 2.02 (s, 6H), 1.76 (d, J = 10.3 Hz, |
| | 1H), 1.52 (d, J = 6.5 Hz, 1H), 1.35 (t, J = 6.9 Hz, 3H), 1.12 (s, |
| | 1H), 1.02 (qd, J = 8.5, 4.3 Hz, 1H), 0.86 (dt, J = 13.0, 6.5 Hz, |
| | 1H), 0.71 (d, J = 6.4 Hz, 3H), 0.65 (d, J = 6.4 Hz, 3H). |
| 1600 | ¹H NMR (400 MHZ, DMSO-d6) 8 13.02 (s, 1H), 8.63 (s, 1H), |
| | 8.54 (s, 2H), 7.91 (s, 1H), 7.64 (s, 2H), 7.25 (d, J = 7.8 Hz, 1H), |
| | 7.13 (d, J = 7.6 Hz, 2H), 6.37 (s, 1H), 5.37 (d, J = 10.0 Hz, |
| | 1H), 4.92 (d, J = 16.5 Hz, 1H), 4.59 (d, J = 16.5 Hz, 1H), 4.18 |
| | (t, J = 11.1 Hz, 1H), 4.11 (t, J = 6.5 Hz, 2H), 4.06 (d, J = 10.6 |
| | Hz, 1H), 2.02 (s, 6H), 1.84 (dt, J = 17.8, 8.4 Hz, 1H), 1.74 (p, |
| | J = 7.1 Hz, 2H), 1.65 (s, 1H), 0.98 (t, J = 7.4 Hz, 5H), 0.31 (s, |
| | 1H), 0.12 (d, J = 10.2 Hz, 2H), -0.25 (s, 2H). |
| 1601 | ¹H NMR (400 MHZ, DMSO-d6) 8 13.09 (s, 1H), 8.75 (s, 1H), |
| | 8.29 (s, 1H), 8.13 (s, 1H), 7.96 (s, 1H), 7.68 (s, 2H), 7.24 (d, J = |
| | 7.9 Hz, 1H), 7.12 (d, J = 7.5 Hz, 2H), 6.38 (d, J = 36.8 Hz, |
| | 1H), 5.43 - 5.30 (m, 2H), 4.86 (d, J = 16.4 Hz, 1H), 4.59 (d, J = |
| | 16.3 Hz, 1H), 4.26 (s, 1H), 4.10 (s, 1H), 2.47 (s, 1H), 2.37 - |
| | 2.29 (m, 1H), 2.22 - 1.86 (m, 8H), 1.74 (qd, J = 9.2, 3.5 Hz, |
| | 3H), 1.42 (d, J = 15.1 Hz, 1H), 0.57 (s, 9H). |
| 1602 | ¹H NMR (400 MHZ, Chloroform-d) 8 8.81 (t, J = 1.9 Hz, 1H), |
| | 8.12 - 8.08 (m, 1H), 8.03 (s, 1H), 7.94 (s, 1H), 7.90 - 7.85 (m, |
| | 1H), 7.67 (t, J = 7.8 Hz, 1H), 7.22 (t, J = 7.6 Hz, 1H), 7.07 (d, |
| | J = 7.6 Hz, 2H), 6.26 (s, 1H), 5.41 (dd, J = 11.0, 4.2 Hz, 1H), |
| | 5.13 (d, J = 15.6 Hz, 1H), 4.77 - 4.68 (m, 1H), 4.23 (d, J = 7.3 |
| | Hz, 1H), 4.15 (d, J = 15.6 Hz, 1H), 4.06 (t, J = 11.4 Hz, 1H), |
| | 3.15 (s, 3H), 3.06 - 2.97 (m, 2H), 2.83 - 2.72 (m, 2H), 2.03 (s, |
| | 6H), 1.71 (dd, J = 15.1, 8.7 Hz, 1H), 1.59 - 1.54 (m, 1H), 0.62 |
| | (s, 9H). Sulfonamide NH not visible. |
| 1603 | ¹H NMR (400 MHZ, Chloroform-d) 8 8.94 (t, J = 1.8 Hz, 1H), |
| | 8.09 (d, J = 8.1 Hz, 1H), 7.95 (s, 1H), 7.89 (s, 1H), 7.85 (dt, J = |
| | 7.6, 1.4 Hz, 1H), 7.65 (t, J = 7.8 Hz, 1H), 7.22 (t, J = 7.6 Hz, |
| | 1H), 7.07 (d, J = 7.6 Hz, 2H), 6.24 (s, 1H), 5.52 (dd, J = 11.1, |
| | 4.1 Hz, 1H), 5.06 (d, J = 15.7 Hz, 1H), 4.29 (p, J = 7.8 Hz, 1H), |
| | 4.24 - 3.98 (m, 4H), 2.52 - 2.41 (m, 2H), 2.09 - 2.00 (m, 8H), |
| | 1.88 - 1.83 (m, 2H), 1.68 (dd, J = 15.2, 8.7 Hz, 2H), 0.63 (s, |
| | 9H). Sulfonamide NH not visible. |
| 1604 | ¹H NMR (400 MHZ, Chloroform-d) 8 8.86 (t, J = 1.9 Hz, 1H), |
| | 8.06 (dt, J = 7.9, 1.5 Hz, 1H), 7.92 (s, 1H), 7.87 (dt, J = 7.6, |
| | 1.4 Hz, 1H), 7.83 (s, 1H), 7.64 (t, J = 7.8 Hz, 1H), 7.21 (t, J = |
| | 7.6 Hz, 1H), 7.07 (d, J = 7.6 Hz, 2H), 6.26 (s, 1H), 5.49 (dd, J = |
| | 11.3, 4.3 Hz, 1H), 5.03 (d, J = 15.4 Hz, 1H), 4.27 - 4.19 (m, |
| | 1H), 4.12 - 3.98 (m, 4H), 2.90 (qd, J = 10.3, 4.0 Hz, 2H), 2.51 |
| | (ddd, J = 8.3, 6.9, 2.0 Hz, 2H), 2.16 - 2.06 (m, 2H), 2.02 (s, |
| | 6H), 1.89 - 1.64 (m, 4H), 0.61 (s, 9H). Sulfonamide NH not |
| | visible |
| 1605 | ¹H NMR (400 MHZ, Chloroform-d) 8 8.77 (s, 1H), 8.07 (d, J = |
| | 7.7 Hz, 1H), 7.97 (s, 1H), 7.84 (dt, J = 7.6, 1.4 Hz, 1H), 7.75 |
| | (s, 1H), 7.65 (t, J = 7.8 Hz, 1H), 7.35 (d, J = 2.0 Hz, 1H), 7.23 |
| | (t, J = 7.6 Hz, 1H), 7.09 (d, J = 7.7 Hz, 2H), 6.30 (s, 1H), 5.83 |
| | (d, J = 2.0 Hz, 1H), 5.42 - 5.30 (m, 2H), 4.91 (d, J = 15.1 Hz, |
| | 1H), 4.17 - 4.11 (m, 1H), 3.97 (d, J = 15.1 Hz, 1H), 3.88 (s, |
| | 3H), 3.86 - 3.81 (m, 1H), 3.73 (t, J = 11.5 Hz, 1H), 3.66 (q, J = |
| | 8.5 Hz, 1H), 2.52 - 2.39 (m, 2H), 2.22 - 2.15 (m, 2H), 2.05 (s, |
| | 6H), 1.59 (dd, J = 15.2, 8.5 Hz, 1H), 1.50 (d, J = 15.1 Hz, 1H), |
| | 0.60 (s, 9H). (Sulfonamide N-H not visible) |
| 1606 | ¹H NMR (400 MHZ, Chloroform-d) 8 8.84 (s, 1H), 8.08 (d, J = |
| | 7.9 Hz, 1H), 7.97 (s, 1H), 7.86 (d, J = 7.6 Hz, 1H), 7.66 (t, J = |
| | 7.8 Hz, 1H), 7.55 (s, 1H), 7.36 (d, J = 1.8 Hz, 1H), 7.22 (t, J = |
| | 7.6 Hz, 1H), 7.08 (d, J = 7.6 Hz, 2H), 6.28 (s, 1H), 5.94 (s, 1H), |
| | 5.34 (d, J = 10.6 Hz, 1H), 5.09 - 4.93 (m, 2H), 4.26 - 4.17 (m, |
| | 1H), 4.14 - 3.97 (m, 3H), 3.92 (s, 3H), 3.72 (q, J = 8.8 Hz, 1H), |
| | 2.36 (q, J = 6.2, 4.6 Hz, 2H), 2.19 - 2.12 (m, 2H), 2.05 (s, 6H), |
| | 1.67 (dd, J = 16.0, 12.0 Hz, 1H), 1.55 (d, J = 15.4 Hz, 1H), |
| | 0.62 (s, 9H). Sulfonamide NH not visible |
| 1607 | ¹H NMR (400 MHz, DMSO-d6) 8 12.95 (s, 1H), 8.69 (s, 1H), |
| | 7.99 (s, 1H), 7.96 (d, J = 6.2 Hz, 1H), 7.89 (s, 1H), 7.69 (s, 2H), |
| | 7.26 (t, J = 7.6 Hz, 1H), 7.12 (d, J = 8.1 Hz, 2H), 6.44 (s, 1H), |
| | 5.34 (dd, J = 10.7, 4.3 Hz, 1H), 4.92 (q, J = 8.3 Hz, 1H), 4.76 |
| | (d, J = 15.9 Hz, 1H), 4.46 (d, J = 16.0 Hz, 1H), 4.30 (t, J = 11.2 |
| | Hz, 1H), 4.18 - 4.04 (m, 2H), 3.17 (s, 3H), 2.95 (s, 3H), 2.25 |
| | (dq, J = 7.7, 3.7 Hz, 1H), 2.22 - 1.82 (m, 8H), 1.82 - 1.69 (m, |
| | 2H), 1.41 (d, J = 15.0 Hz, 1H), 0.56 (s, 9H). |
| 1608 | ¹H NMR (400 MHZ, DMSO-d6) 8 12.88 (s, 1H), 8.69 (s, 1H), |
| | 8.00 (s, 1H), 7.96 (d, J = 7.3 Hz, 1H), 7.89 (s, 1H), 7.69 (d, J = |
| | 8.4 Hz, 2H), 7.26 (t, J = 7.6 Hz, 1H), 7.12 (d, J = 7.6 Hz, 2H), |
| | 6.44 (s, 1H), 5.43 (dd, J = 10.7, 4.3 Hz, 1H), 4.95 (q, J = 8.1 |
| | Hz, 1H), 4.72 (d, J = 16.0 Hz, 1H), 4.48 (d, J = 15.9 Hz, 1H), |
| | 4.31 (t, J = 10.9 Hz, 1H), 4.24 (td, J = 5.6, 2.8 Hz, 1H), 4.06 |
| | (s, 1H), 3.17 (s, 3H), 3.06 (s, 3H), 2.26 (ddd, J = 8.3, 4.9, 2.7 |
| | Hz, 1H), 2.24 - 1.67 (m, 10H), 1.42 (d, J = 14.9 Hz, 1H), 0.56 |
| | (s, 9H). |
| 1611 | ¹H NMR (400 MHZ, DMSO-d6) 8 12.85 (s, 1H), 8.66 (s, 1H), |
| | 8.00 (s, 1H), 7.95 (d, J = 6.9 Hz, 1H), 7.89 (s, 1H), 7.68 (d, J = |
| | 6.5 Hz, 2H), 7.26 (t, J = 7.6 Hz, 1H), 7.12 (d, J = 7.6 Hz, 2H), |
| | 6.43 (s, 1H), 5.37 (dd, J = 10.8, 4.3 Hz, 1H), 4.74 (d, J = 15.9 |
| | Hz, 1H), 4.51 - 4.41 (m, 2H), 4.29 (t, J = 11.1 Hz, 1H), 4.07 (d, |
| | J = 10.3 Hz, 1H), 3.62 (p, J = 6.9 Hz, 1H), 3.17 (d, J = 5.2 Hz, |
| | 1H), 3.13 (s, 3H), 3.04 (s, 3H), 2.58 (qd, J = 13.5, 5.9 Hz, 2H), |
| | 2.12 - 1.90 (m, 7H), 1.76 (dd, J = 15.3, 8.9 Hz, 1H), 1.42 (d, J = |
| | 15.0 Hz, 1H), 0.56 (s, 9H). |
| 1612 | ¹H NMR (400 MHZ, DMSO-d6) & 13.08 (s, 1H), 8.46 (s, 1H), |
| | 8.18 (d, J = 1.4 Hz, 1H), 8.12 (d, J = 1.5 Hz, 1H), 7.93 (d, J = |
| | 6.4 Hz, 1H), 7.67 (d, J = 6.0 Hz, 2H), 7.26 (t, J = 7.6 Hz, 1H), |
| | 7.13 (d, J = 7.7 Hz, 2H), 6.43 (s, 1H), 5.22 (dd, J = 10.9, 4.4 |
| | Hz, 1H), 4.83 - 4.74 (m, 1H), 4.71 (d, J = 15.1 Hz, 1H), 4.42 - |
| | 4.30 (m, 2H), 3.98 (td, J = 10.6, 8.9, 4.3 Hz, 1H), 3.00 (s, 3H), |
| | 2.22 - 2.10 (m, 5H), 2.09 - 1.82 (m, 6H), 1.71 - 1.59 (m, 2H), |
| | 1.40 (d, J = 15.0 Hz, 1H), 0.54 (s, 9H). |
| 1613 | ¹H NMR (400 MHZ, DMSO-d6) 8 13.37 - 11.71 (broad m, 1H), |
| | 8.64 (s, 1H), 8.02 (s, 1H), 7.92 (s, 1H), 7.87 (br s, 1H), 7.64 (br |
| | s, 2H), 7.23 (br s, 1H), 7.11 (br s, 2H), 6.40 (br s, 1H), 5.43 - |
| | 5.24 (m, 1H), 4.70 (d, J = 15.8 Hz, 1H), 4.60 (d, J = 5.8 Hz, |
| | 1H), 4.55 (d, J = 5.9 Hz, 1H), 4.43 (d, J = 15.5 Hz, 1H), 4.11 |
| | (dd, J = 11.9, 5.8 Hz, 4H), 3.96 (d, J = 14.8 Hz, 1H), 3.81 (d, J = |
| | 14.6 Hz, 1H), 3.07 (s, 3H), 2.16 - 1.86 (m, 6H), 1.78 - 1.66 |
| | (m, 1H), 1.44 - 1.36 (m, 1H), 1.18 (s, 3H), 0.55 (s, 9H). |
| 1614 | ¹H NMR (400 MHZ, DMSO-d6) 8 13.90-11.64 (broad m, 1H), |
| | 8.65 (s, 1H), 8.02 - 7.90 (m, 2H), 7.84 (s, 1H), 7.68 (s, 2H), 7.26 |
| | (t, J = 7.6 Hz, 1H), 7.12 (d, J = 7.6 Hz, 2H), 6.44 (s, 1H), 5.39 |
| | (dd, J = 10.9, 4.3 Hz, 1H), 4.71 (d, J = 15.8 Hz, 1H), 4.43 (d, J = |
| | 15.7 Hz, 1H), 4.32 (t, J = 11.2 Hz, 1H), 4.10 - 3.98 (m, 1H), |
| | 3.69 (overlapped with water, dd, J = 14.3, 7.1 Hz, 1H), 3.60 |
| | (overlapped with water, dd, J = 14.3, 7.3 Hz, 1H), 3.09 (s, 3H), |
| | 2.71 - 2.57 (m, 1H), 2.21 - 1.84 (m, 8H), 1.81 - 1.63 (m, 5H), |
| | 1.41 (d, J = 14.9 Hz, 1H), 0.56 (s, 9H). |
| 1615 | ¹H NMR (400 MHZ, DMSO-d6) 8 12.77 (s, 1H), 8.60 (s, 1H), |
| | 7.93 (d, J = 4.8 Hz, 2H), 7.89 (s, 1H), 7.67 (d, J = 6.2 Hz, 2H), |
| | 7.26 (t, J = 7.6 Hz, 1H), 7.12 (d, J = 7.6 Hz, 2H), 6.43 (s, 1H), |
| | 5.42 (dd, J = 10.9, 4.3 Hz, 1H), 5.09 (p, J = 8.1 Hz, 1H), 4.70 |
| | (d, J = 15.5 Hz, 1H), 4.41 (d, J = 15.6 Hz, 1H), 4.34 (d, J = |
| | 11.3 Hz, 1H), 4.05 (td, J = 12.4, 11.2, 4.7 Hz, 1H), 3.92 (dq, J = |
| | 6.9, 2.9 Hz, 1H), 3.09 (s, 3H), 3.07 (s, 3H), 2.45 - 2.30 (m, |
| | 3H), 2.25 (dddd, J = 12.2, 10.2, 4.8, 2.2 Hz, 1H), 2.01 (s, 6H), |
| | 1.76 (dd, J = 15.2, 8.8 Hz, 1H), 1.43 (d, J = 15.0 Hz, 1H), 0.56 |
| | (s, 9H). |
| 1616 | ¹H NMR (400 MHZ, DMSO-d6) 8 13.05 (s, 1H), 8.76 (s, 1H), |
| | 8.28 (d, J = 5.0 Hz, 1H), 7.96 (s, 1H), 7.68 (s, 2H), 7.25 (d, J = |
| | 7.9 Hz, 1H), 7.12 (d, J = 7.4 Hz, 2H), 6.64 (d, J = 5.0 Hz, 1H), |
| | 6.42 (s, 1H), 5.41 (d, J = 8.6 Hz, 1H), 5.24 (p, J = 8.7 Hz, 1H), |
| | 4.64 (d, J = 16.6 Hz, 1H), 4.44 (d, J = 16.7 Hz, 1H), 4.29 - 4.19 |
| | (m, 1H), 4.09 (dd, J = 6.8, 3.7 Hz, 1H), 3.12 (s, 3H), 2.23 - 1.88 |
| | (m, 10H), 1.75 - 1.60 (m, 3H), 1.40 (d, J = 15.1 Hz, 1H), 0.55 |
| | (s, 9H). |
| 1617 | ¹H NMR (400 MHZ, DMSO-d6) 8 13.06 (br. s, 1H), 8.66 (br. s, |
| | 1H), 7.95 (br. s, 1H), 7.70 (br. s, 2H), 7.44 (t, J = 7.8 Hz, 1H), |
| | 7.31 - 7.19 (m, 1H), 7.17 - 7.07 (m, 2H), 6.62 (d, J = 7.1 Hz, |
| | 1H), 6.40 (br. s, 1H), 6.35 (d, J = 8.3 Hz, 1H), 5.15 (d, J = 6.6 |
| | Hz, 1H), 4.81 (d, J = 15.4 Hz, 1H), 4.32 (d, J = 15.7 Hz, 1H), |
| | 4.17 - 4.00 (m, 3H), 3.50 (d, J = 9.0 Hz, 1H), 3.42 - 3.34 (m, |
| | 2H), 3.00 (s, 3H), 2.47 - 2.39 (m, 1H, overlapped with DMSO), |
| | 2.20 - 1.80 (m, 11H), 1.77 - 1.67 (m, 1H), 1.35 (d, J = 14.9 Hz, |
| | 1H), 0.70 (t, J = 7.3 Hz, 3H), 0.53 (s, 9H). |
| 1618 | ¹H NMR (400 MHZ, DMSO-d6) & 13.04 (br. s, 1H), 8.63 (br. s, |
| | 1H), 7.95 (br. s, 1H), 7.69 (br. s, 2H), 7.43 (t, J = 7.8 Hz, 1H), |
| | 7.30 - 7.21 (m, 1H), 7.17 - 7.06 (m, 2H), 6.61 (d, J = 7.1 Hz, |
| | 1H), 6.41 (br. s, 1H), 6.37 (d, J = 8.3 Hz, 1H), 5.29 - 5.22 (m, |
| | 1H), 4.72 (d, J = 15.4 Hz, 1H), 4.32 (d, J = 15.4 Hz, 1H), 4.22 - |
| | 4.13 (m, 1H), 4.12 - 4.04 (m, 1H), 3.96 (d, J = 9.0 Hz, 1H), |
| | 3.65 (d, J = 9.0 Hz, 1H), 3.47 - 3.41 (m, 1H), 3.40 - 3.34 (m, |
| | 1H), 3.18 (s, 3H), 2.48 - 2.42 (m, 1H, overlapped with DMSO), |
| | 2.11 - 1.76 (m, 11H), 1.70 - 1.58 (m, 1H), 1.37 (d, J = 14.9 Hz, |
| | 1H), 0.58 (t, J = 7.3 Hz, 3H), 0.52 (s, 9H). |
| 1619 | ¹H NMR (400 MHZ, DMSO-d6) 8 12.77 (s, 1H), 8.68 (d, J = |
| | 2.2 Hz, 1H), 7.99 (s, 1H), 7.98 - 7.92 (m, 1H), 7.87 (s, 1H), 7.69 |
| | (d, J = 6.8 Hz, 2H), 7.26 (t, J = 7.6 Hz, 1H), 7.12 (d, J = 7.7 |
| | Hz, 2H), 6.43 (s, 1H), 5.40 (dd, J = 10.8, 4.3 Hz, 1H), 4.76 - |
| | 4.65 (m, 2H), 4.45 (d, J = 15.9 Hz, 1H), 4.29 (t, J = 11.1 Hz, |
| | 1H), 4.10 - 4.01 (m, 1H), 3.33 (s, 1H), 3.23 (s, 3H), 3.04 (s, |
| | 3H), 2.26 (tdd, J = 16.1, 12.1, 6.2 Hz, 4H), 2.14 - 1.87 (m, 8H), |
| | 1.76 (dd, J = 15.2, 9.0 Hz, 1H), 1.42 (d, J = 14.9 Hz, 1H), 0.56 |
| | (s, 9H). |
| 1620 | ¹H NMR (400 MHZ, DMSO-d6) & 12.79 (s, 1H), 8.60 (s, 1H), |
| | 7.96 (d, J = 9.6 Hz, 2H), 7.88 (d, J = 2.8 Hz, 1H), 7.68 (s, 2H), |
| | 7.25 (d, J = 8.0 Hz, 1H), 7.12 (d, J = 7.7 Hz, 2H), 6.46 (s, 1H), |
| | 5.43 (d, J = 10.8 Hz, 1H), 5.14 (d, J = 10.5 Hz, 1H), 4.69 (d, J = |
| | 15.2 Hz, 1H), 4.40 (d, J = 14.8 Hz, 2H), 4.07 (d, J = 12.0 Hz, |
| | 1H), 3.39 (d, J = 5.0 Hz, 2H), 3.28 - 3.21 (m, 1H), 3.19 - 3.10 |
| | (m, 3H), 3.10 - 3.01 (m, 3H), 2.33 (s, 3H), 2.06 (d, J = 31.6 Hz, |
| | 8H), 1.76 (t, J = 12.3 Hz, 1H), 1.44 (d, J = 15.1 Hz, 1H), 0.56 |
| | (d, J = 2.6 Hz, 9H). |
| 1623 | ¹H NMR (400 MHZ, DMSO-d6) & 12.79 (s, 1H), 8.84 (s, 1H), |
| | 7.96 (d, J = 7.4 Hz, 1H), 7.88 (s, 1H), 7.71 (dd, J = 13.4, 5.8 |
| | Hz, 2H), 7.50 - 7.43 (m, 3H), 7.37 (d, J = 8.0 Hz, 2H), 7.26 (t, |
| | J = 7.6 Hz, 1H), 7.12 (d, J = 7.7 Hz, 2H), 6.57 (d, J = 4.8 Hz, |
| | 1H), 6.33 (s, 1H), 4.71 (p, J = 8.6 Hz, 1H), 4.28 (t, J = 6.1 Hz, |
| | 1H), 3.82 (d, J = 16.9 Hz, 1H), 3.41 (s, 1H), 2.94 (s, 3H), 2.19 |
| | - 2.07 (m, 4H), 2.06 (s, 6H), 1.60 (dt, J = 18.5, 9.0 Hz, 2H), |
| | 1.28 (s, 9H), 1.17 (d, J = 6.9 Hz, 3H). |
| 1314 | ¹H NMR (400 MHZ, DMSO-d6) 8 13.00 (s, 1H), 8.76 (s, 1H), |
| | 7.94 (s, 1H), 7.87 (s, 1H), 7.77 (s, 1H), 7.67 (s, 2H), 7.24 (d, J = |
| | 7.9 Hz, 1H), 7.12 (d, J = 7.6 Hz, 2H), 6.42 (s, 1H), 5.51 (d, J = |
| | 9.8 Hz, 1H), 4.73 (d, J = 16.1 Hz, 1H), 4.54 (s, 1H), 4.46 (d, |
| | J = 16.1 Hz, 1H), 4.30 - 4.18 (m, 1H), 4.07 - 3.94 (m, 2H), 3.66 |
| | (q, J = 9.8, 9.2 Hz, 1H), 3.23 - 3.16 (m, 1H), 2.45 - 2.38 (m, |
| | 1H), 2.17 - 1.90 (m, 8H), 1.85 (dd, J = 13.0, 6.9 Hz, 1H), 1.72 |
| | (dd, J = 15.9, 8.8 Hz, 2H), 1.63 (d, J = 12.6 Hz, 1H), 1.42 (d, J = |
| | 15.0 Hz, 1H), 0.57 (s, 9H). |
| 1624 | ¹H NMR (400 MHZ, Chloroform-d) 8 8.86 (t, J = 1.8 Hz, 1H), |
| | 8.08 (dt, J = 8.0, 1.4 Hz, 1H), 8.04 (s, 1H), 7.97 (s, 1H), 7.87 |
| | (dd, J = 7.6, 1.4 Hz, 1H), 7.65 (t, J = 7.7 Hz, 1H), 7.22 (t, J = |
| | 7.6 Hz, 1H), 7.08 (d, J = 7.6 Hz, 2H), 6.28 (s, 1H), 5.68 (dd, J = |
| | 11.2, 4.1 Hz, 1H), 5.35 (d, J = 7.7 Hz, 2H), 4.98 (d, J = 15.3 |
| | Hz, 1H), 4.83 (d, J = 7.8 Hz, 1H), 4.77 (d, J = 7.8 Hz, 1H), 4.31 - |
| | 4.22 (m, 1H), 4.13 (d, J = 14.0 Hz, 2H), 4.02 (q, J = 7.2 Hz, |
| | 2H), 2.69 - 2.62 (m, 2H), 2.04 (s, 6H), 1.69 (dd, J = 15.2, 8.6 |
| | Hz, 1H), 1.56 (d, J = 15.1 Hz, 1H), 0.61 (s, 9H). Sulfonamide |
| | NH not visible. |
| 1625 | ¹H NMR (400 MHZ, DMSO-d6) 8 8.64 (s, 1H), 7.98 (s, 1H), |
| | 7.95 (t, J = 5.0 Hz, 1H), 7.90 (s, 1H), 7.69 (d, J = 4.5 Hz, 2H), |
| | 7.26 (t, J = 7.6 Hz, 1H), 7.12 (d, J = 7.7 Hz, 2H), 6.44 (s, 1H), |
| | 5.35 (dd, J = 10.7, 4.4 Hz, 1H), 4.74 (d, J = 15.5 Hz, 1H), 4.44 |
| | (d, J = 15.6 Hz, 1H), 4.30 (t, J = 11.2 Hz, 1H), 4.09 (d, J = 8.0 |
| | Hz, 1H), 3.52 (d, J = 6.5 Hz, 2H), 2.29 - 2.21 (m, 2H), 2.22 - |
| | 2.15 (m, 2H), 2.15 - 1.68 (m, 13H), 1.41 (d, J = 15.0 Hz, 1H), |
| | 1.19 (d, J = 35.1 Hz, 1H), 0.55 (s, 9H). |
| 1626 | ¹H NMR (400 MHZ, DMSO-d6) 8 12.91 (s, 1H), 8.61 (s, 1H), |
| | 7.93 (d, J = 12.6 Hz, 2H), 7.83 (s, 1H), 7.67 (s, 2H), 7.26 (t, J = |
| | 7.6 Hz, 1H), 7.12 (d, J = 7.6 Hz, 2H), 6.43 (s, 1H), 5.31 (dd, |
| | J = 10.6, 4.3 Hz, 1H), 4.71 (d, J = 15.8 Hz, 1H), 4.45 (d, J = |
| | 15.8 Hz, 1H), 4.27 (t, J = 11.1 Hz, 1H), 4.04 (t, J = 11.5 Hz, |
| | 1H), 3.89 (d, J = 13.7 Hz, 1H), 3.51 (t, J = 11.0 Hz, 1H), 2.37 - |
| | 2.27 (m, 2H), 2.19 (dd, J = 16.3, 10.2 Hz, 2H), 2.05 (d, J = |
| | 67.5 Hz, 6H), 1.83 - 1.77 (m, 2H), 1.76 - 1.58 (m, 5H), 1.40 (d, |
| | J = 15.0 Hz, 1H), 1.30 (d, J = 3.5 Hz, 1H), 1.22 - 1.13 (m, 1H), |
| | 0.54 (s, 9H). |
| 1627 | ¹H NMR (400 MHZ, DMSO-d6) 8 13.02 (s, 1H), 8.60 (d, J = |
| | 12.3 Hz, 1H), 8.15 - 7.89 (m, 3H), 7.68 (d, J = 6.3 Hz, 2H), |
| | 7.26 (t, J = 7.6 Hz, 1H), 7.12 (d, J = 7.6 Hz, 2H), 6.44 (s, 1H), |
| | 5.28 (dd, J = 10.7, 4.3 Hz, 1H), 4.75 (d, J = 15.8 Hz, 1H), 4.49 |
| | (d, J = 15.9 Hz, 1H), 4.27 (t, J = 11.1 Hz, 1H), 4.08 - 3.96 (m, |
| | 1H), 3.81 (ddd, J = 14.0, 5.9, 2.9 Hz, 1H), 3.73 (d, J = 4.9 Hz, |
| | 1H), 3.65 (ddd, J = 13.9, 7.1, 2.9 Hz, 1H), 3.49 (ddd, J = 11.4, |
| | 5.8, 2.9 Hz, 1H), 3.40 (td, J = 7.9, 7.5, 3.5 Hz, 1H), 2.38 - 2.29 |
| | (m, 1H), 2.29 - 2.14 (m, 3H), 1.95 (s, 6H), 1.73 (ddd, J = 16.6, |
| | 14.0, 9.4 Hz, 3H), 1.41 (d, J = 15.0 Hz, 1H), 0.56 (d, J = 8.7 |
| | Hz, 9H). |
| 1316 | ¹H NMR (400 MHZ, DMSO-d6) 8 13.41 - 11.42 (broad m, 1H), |
| | 8.75 (s, 1H), 7.99 - 7.82 (m, 3H), 7.68 (br s, 2H), 7.25 (t, J = |
| | 7.6 Hz, 1H), 7.12 (d, J = 7.6 Hz, 2H), 6.43 (br s, 1H), 5.40 - |
| | 5.25 (m, 1H), 4.78 (d, J = 16.3 Hz, 1H), 4.60 - 4.40 (m, 2H), |
| | 4.29 - 4.15 (m, 1H), 4.14 - 4.00 (m, 1H), 3.94 - 3.80 (m, 1H), |
| | 3.25 - 3.04 (m, 1H), 2.64 - 2.55 (m, 1H), 2.26 - 1.81 (m, 11H), |
| | 1.75 (dd, J = 15.2, 8.9 Hz, 1H), 1.62 - 1.48 (m, 1H), 1.47 - 1.31 |
| | (m, 3H), 0.55 (s, 9H). |
| 1315 | ¹H NMR (400 MHZ, DMSO-d6) 8 13.62 - 11.53 (broad m, 1H), |
| | 8.70 (s, 1H), 8.01 - 7.85 (m, 3H), 7.67 (br s, 2H), 7.33 - 7.19 |
| | (m, 1H), 7.17 - 7.03 (m, 2H), 6.44 (br s, 1H), 5.52 - 5.35 (m, |
| | 1H), 4.70 (d, J = 16.0 Hz, 1H), 4.58 - 4.40 (m, 2H), 4.31 (t, J = |
| | 11.2 Hz, 1H), 4.09 - 3.98 (m, 1H), 3.93 (d, J = 12.7 Hz, 1H), |
| | 3.17 (t, J = 10.8 Hz, 1H), 2.63 (s, 1H), 2.30 - 1.83 (m, 11H), |
| | 1.74 (dd, J = 15.2, 9.0 Hz, 1H), 1.60 - 1.38 (m, 4H), 0.56 (s, |
| | 9H). |
| 1628 | ¹H NMR (400 MHZ, DMSO-d6) 8 13.49 - 11.88 (broad m, 1H), |
| | 8.70 (s, 1H), 7.94 (s, 1H), 7.90 (s, 1H), 7.74 (s, 1H), 7.67 (s, |
| | 2H), 7.30 - 7.21 (m, 1H), 7.17 - 7.04 (m, 2H), 6.41 (s, 1H), 5.38 |
| | (d, J = 9.9 Hz, 1H), 5.06 (t, J = 5.0 Hz, 1H), 4.87 (td, J = 5.3, |
| | 2.2 Hz, 1H), 4.77 (d, J = 16.3 Hz, 1H), 4.45 (d, J = 16.2 Hz, |
| | 1H), 4.32 - 4.09 (m, 3H), 4.08 - 3.96 (m, 1H), 3.94 - 3.76 (m, |
| | 2H), 2.24 - 1.80 (m, 7H), 1.79 - 1.62 (m, 2H), 1.41 (d, J = 14.9 |
| | Hz, 1H), 0.56 (s, 9H). |
| 1629 | ¹H NMR (400 MHZ, DMSO-d6) 8 13.48 - 11.54 (broad m, 1H), |
| | 8.67 (s, 1H), 7.99 - 7.87 (m, 2H), 7.73 (s, 1H), 7.66 (s, 2H), 7.31 - |
| | 7.19 (m, 1H), 7.12 (d, J = 7.7 Hz, 2H), 6.41 (s, 1H), 5.53 (d, |
| | J = 10.3 Hz, 1H), 5.06 (t, J = 5.1 Hz, 1H), 4.88 (td, J = 5.4, 2.1 |
| | Hz, 1H), 4.71 (d, J = 16.0 Hz, 1H), 4.45 (d, J = 16.0 Hz, 1H), |
| | 4.34 - 4.22 (m, 1H), 4.23 - 4.10 (m, 2H), 4.07 - 3.90 (m, 2H), |
| | 3.89 - 3.79 (m, 1H), 2.28 - 1.78 (m, 8H), 1.72 (dd, J = 15.2, 9.0 |
| | Hz, 1H), 1.43 (d, J = 14.9 Hz, 1H), 0.57 (s, 9H). |
| 1630 | ¹H NMR (400 MHZ, Chloroform-d) 8 8.73 (t, J = 1.9 Hz, 1H), |
| | 8.10 (d, J = 7.9 Hz, 1H), 7.94 (s, 1H), 7.90 - 7.83 (m, 2H), 7.68 |
| | (t, J = 7.8 Hz, 1H), 7.25 (t, J = 7.8 Hz, 1H), 7.10 (d, J = 7.7 |
| | Hz, 2H), 6.33 (s, 1H), 5.44 (d, J = 10.4 Hz, 1H), 5.02 (d, J = |
| | 15.3 Hz, 1H), 4.64 (s, 1H), 4.35 (d, J = 17.0 Hz, 1H), 4.20 (d, |
| | J = 14.1 Hz, 2H), 4.11 (t, J = 11.3 Hz, 1H), 3.29 (s, 3H), 2.98 |
| | (s, 3H), 2.93 (s, 3H), 2.07 (s, 6H), 1.65 (dd, J = 15.4, 8.6 Hz, |
| | 1H), 1.55 (d, J = 12.0 Hz, 1H), 0.62 (s, 9H). Sulfonamide N-H |
| | not visible. |
| 1636 | ¹H NMR (400 MHZ, DMSO-d6) 8 12.88 (s, 1H), 8.66 (s, 1H), |
| | 7.94 (d, J = 5.4 Hz, 3H), 7.87 (s, 1H), 7.69 (d, J = 5.4 Hz, 2H), |
| | 7.55 (s, 1H), 7.26 (t, J = 7.6 Hz, 1H), 7.13 (d, J = 7.7 Hz, 2H), |
| | 6.44 (s, 1H), 5.38 (dd, J = 10.8, 4.3 Hz, 1H), 4.77 (d, J = 15.8 |
| | Hz, 1H), 4.48 (d, J = 15.8 Hz, 1H), 4.31 (t, J = 11.2 Hz, 1H), |
| | 4.04 (q, J = 9.7, 6.8 Hz, 1H), 3.81 (s, 3H), 3.40 (s, 3H), 1.95 (d, |
| | J = 35.8 Hz, 6H), 1.76 (dd, J = 15.2, 8.9 Hz, 1H), 1.44 (d, J = |
| | 14.9 Hz, 1H), 0.57 (s, 9H). |
| 1637 | ¹H NMR (400 MHZ, DMSO-d6) 8 12.99 (s, 1H), 8.68 (s, 1H), |
| | 7.94 (d, J = 6.6 Hz, 1H), 7.90 (s, 1H), 7.68 (s, 3H), 7.26 (t, J = |
| | 7.6 Hz, 1H), 7.12 (d, J = 7.6 Hz, 2H), 6.41 (s, 1H), 5.34 (d, J = |
| | 9.4 Hz, 1H), 4.77 (d, J = 16.0 Hz, 1H), 4.43 (d, J = 16.5 Hz, |
| | 1H), 4.37 (dd, J = 8.4, 5.9 Hz, 1H), 4.18 (t, J = 11.1 Hz, 1H), |
| | 4.06 (td, J = 9.9, 9.4, 5.8 Hz, 2H), 3.94 - 3.85 (m, 1H), 2.92 - |
| | 2.78 (m, 1H), 2.33 (d, J = 22.3 Hz, 1H), 2.23 - 2.12 (m, 2H), |
| | 1.89 (dt, J = 13.9, 7.1 Hz, 9H), 1.83 - 1.67 (m, 3H), 1.41 (d, J = |
| | 14.9 Hz, 1H), 0.56 (s, 9H). |
| 1638 | ¹H NMR (400 MHZ, DMSO-d6) & 13.01 (s, 1H), 8.63 (s, 1H), |
| | 7.94 (s, 1H), 7.91 (s, 1H), 7.68 (d, J = 9.3 Hz, 3H), 7.26 (t, J = |
| | 7.6 Hz, 1H), 7.12 (d, J = 7.7 Hz, 2H), 6.43 (s, 1H), 5.47 (d, J = |
| | 10.1 Hz, 1H), 4.71 (d, J = 15.9 Hz, 1H), 4.44 (d, J = 15.8 Hz, |
| | 1H), 4.39 (dd, J = 8.5, 5.8 Hz, 1H), 4.27 (t, J = 11.1 Hz, 1H), |
| | 4.09 (q, J = 8.6 Hz, 1H), 4.02 - 3.88 (m, 2H), 2.94 - 2.83 (m, |
| | 1H), 2.39 (t, J = 7.6 Hz, 2H), 2.16 (dt, J = 10.3, 5.7 Hz, 2H), |
| | 1.94 (p, J = 8.1 Hz, 8H), 1.88 - 1.81 (m, 1H), 1.73 (dd, J = 15.2, |
| | 8.7 Hz, 2H), 1.42 (d, J = 14.9 Hz, 1H), 0.57 (s, 9H). |
| 1317 | ¹H NMR (400 MHZ, DMSO-d6) 8 13.37 - 11.64 (broad m, 1H), |
| | 8.65 (s, 1H), 8.52 (s, 2H), 7.94 (s, 1H), 7.75 - 7.46 (broad m, |
| | 2H), 7.33 - 7.20 (m, 1H), 7.12 (d, J = 7.8 Hz, 2H), 6.43 (br s, |
| | 1H), 5.42 - 5.29 (m, 1H), 4.88 (d, J = 16.5 Hz, 1H), 4.80 (h, J = |
| | 6.0 Hz, 1H), 4.69 (d, J = 16.6 Hz, 1H), 4.20 (t, J = 11.3 Hz, |
| | 1H), 4.09 - 3.96 (m, 1H), 2.24 - 1.86 (m, 7H), 1.81 - 1.67 (m, |
| | 2H), 1.65 - 1.57 (m, 1H), 1.51 (d, J = 14.9 Hz, 1H), 1.47 - 1.39 |
| | (m, 1H), 1.34 - 1.26 (m, 8H), 0.60 (s, 3H). |
| 1641 | ¹H NMR (400 MHZ, DMSO-d6) 8 13.36 - 11.56 (broad m, 1H), |
| | 8.65 (s, 1H), 8.54 (s, 2H), 7.93 (s, 1H), 7.76 - 7.52 (br m, 2H), |
| | 7.36 - 7.20 (m, 1H), 7.12 (d, J = 7.6 Hz, 2H), 6.43 (br s, 1H), |
| | 5.36 (d, J = 9.2 Hz, 1H), 4.88 (d, J = 16.5 Hz, 1H), 4.69 (d, J = |
| | 16.6 Hz, 1H), 4.19 (t, J = 11.2 Hz, 1H), 4.11 (t, J = 6.5 Hz, |
| | 2H), 4.04 (d, J = 11.0 Hz, 1H), 2.24 - 1.85 (m, 7H), 1.82 - 1.67 |
| | (m, 4H), 1.66 - 1.55 (m, 1H), 1.51 (d, J = 14.8 Hz, 1H), 1.47 - |
| | 1.39 (m, 1H), 1.34 - 1.26 (m, 2H), 0.98 (t, J = 7.4 Hz, 3H), 0.60 |
| | (s, 3H). |
| 1642 | ¹H NMR (400 MHZ, DMSO-d6) 8 12.82 (s, 1H), 8.74 (s, 1H), |
| | 7.98 - 7.91 (m, 1H), 7.88 (d, J = 10.2 Hz, 2H), 7.67 (d, J = 5.6 |
| | Hz, 2H), 7.25 (t, J = 7.6 Hz, 1H), 7.12 (d, J = 7.6 Hz, 2H), 6.40 |
| | (s, 1H), 5.41 (dd, J = 10.8, 4.2 Hz, 1H), 4.78 (d, J = 16.1 Hz, |
| | 1H), 4.67 - 4.57 (m, 2H), 4.45 (d, J = 16.1 Hz, 1H), 4.23 (t, J = |
| | 11.2 Hz, 1H), 4.17 - 4.02 (m, 2H), 3.79 (t, J = 9.3 Hz, 1H), 3.72 - |
| | 3.58 (m, 2H), 3.43 (td, J = 10.5, 6.8 Hz, 1H), 2.25 - 1.93 (m, |
| | 8H), 1.85 (dddd, J = 12.5, 6.1, 4.3, 1.5 Hz, 1H), 1.75 (dd, J = |
| | 15.2, 8.9 Hz, 1H), 1.41 (d, J = 15.0 Hz, 1H), 0.56 (s, 9H). |
| 1643 | ¹H NMR (400 MHZ, DMSO-d6) 8 12.92 (s, 1H), 8.73 (s, 1H), |
| | 7.94 (d, J = 7.0 Hz, 1H), 7.88 (d, J = 9.0 Hz, 2H), 7.67 (d, J = |
| | 7.9 Hz, 2H), 7.25 (t, J = 7.6 Hz, 1H), 7.12 (d, J = 7.6 Hz, 2H), |
| | 6.41 (s, 1H), 5.55 (dd, J = 10.9, 4.2 Hz, 1H), 4.76 - 4.63 (m, |
| | 2H), 4.62 - 4.56 (m, 1H), 4.48 (d, J = 16.0 Hz, 1H), 4.32 (d, J = |
| | 11.5 Hz, 1H), 4.02 (s, 1H), 3.77 (ddd, J = 12.5, 7.7, 3.5 Hz, |
| | 2H), 3.67 (td, J = 8.4, 6.3 Hz, 1H), 3.41 (td, J = 10.3, 6.8 Hz, |
| | 1H), 2.37 - 2.25 (m, 1H), 2.23 - 1.79 (m, 9H), 1.73 (dd, J = |
| | 15.2, 9.0 Hz, 1H), 1.44 (d, J = 14.9 Hz, 1H), 0.58 (s, 9H). |
| 1318 | ¹H NMR (400 MHZ, DMSO-d6) 8 13.03 (br. s, 1H), 8.51 (br. s, |
| | 1H), 7.92 (br. s, 1H), 7.76 - 7.56 (m, 2H), 7.43 (s, 1H), 7.30 - |
| | 7.21 (m, 1H), 7.19 - 7.05 (m, 2H), 6.40 (br. s, 1H), 5.24 (dd, J = |
| | 10.8, 4.2 Hz, 1H), 4.53 (d, J = 15.4 Hz, 1H), 4.44 - 4.27 (m, |
| | 2H), 4.17 (d, J = 15.7 Hz, 1H), 3.93 - 3.81 (m, 1H), 2.94 (s, |
| | 3H), 2.27 - 2.06 (m, 6H), 2.04 - 1.81 (m, 5H), 1.69 - 1.58 (m, |
| | 2H), 1.37 (d, J = 15.2 Hz, 1H), 0.52 (s, 9H). |
| 1645 | ¹H NMR (400 MHz, DMSO-d6) 8 13.04 (br. s, 1H), 8.51 (br. s, |
| | 1H), 7.92 (br. s, 1H), 7.74 - 7.58 (m, 2H), 7.43 (s, 1H), 7.30 - |
| | 7.22 (m, 1H), 7.18 - 7.05 (m, 2H), 6.40 (br. s, 1H), 5.24 (dd, J = |
| | 10.6, 4.0 Hz, 1H), 4.53 (d, J = 15.2 Hz, 1H), 4.37 - 4.29 (m, |
| | 1H), 4.28 - 4.22 (m, 1H), 4.17 (d, J = 15.4 Hz, 1H), 3.94 - 3.84 |
| | (m, 1H), 3.49 - 3.36 (m, 2H), 2.25 - 2.12 (m, 5H), 2.10 - 1.82 |
| | (m, 6H), 1.71 - 1.56 (m, 2H), 1.37 (d, J = 14.9 Hz, 1H), 1.10 (t, |
| | J = 7.0 Hz, 3H), 0.51 (s, 9H). |
| 1646 | ¹H NMR (400 MHZ, DMSO-d6) 8 13.00 (br. s, 1H), 8.51 (br. s, |
| | 1H), 7.92 (br. s, 1H), 7.66 (br. s, 2H), 7.41 (s, 1H), 7.29 - 7.22 |
| | (m, 1H), 7.17 - 7.08 (m, 2H), 6.40 (br. s, 1H), 5.22 (dd, J = |
| | 10.9, 4.0 Hz, 1H), 4.51 (d, J = 15.4 Hz, 1H), 4.31 (t, J = 11.1 |
| | Hz, 1H), 4.18 (d, J = 15.7 Hz, 1H), 3.92 - 3.80 (m, 1H), 3.45 - |
| | 3.34 (m, 2H), 2.97 (s, 3H), 2.68 - 2.58 (m, 1H), 2.19 - 1.88 (m, |
| | 9H), 1.86 - 1.78 (m, 2H), 1.78 - 1.67 (m, 2H), 1.37 (d, J = 14.9 |
| | Hz, 1H), 0.51 (s, 9H). |
| 1647 | ¹H NMR (400 MHZ, DMSO-d6) 8 13.06 (br. s, 1H), 8.49 (br. s, |
| | 1H), 7.93 (br. s, 1H), 7.66 (br. s, 2H), 7.51 (s, 1H), 7.31 - 7.22 |
| | (m, 1H), 7.17 - 7.07 (m, 2H), 6.40 (br. s, 1H), 5.22 (dd, J = |
| | 10.9, 4.3 Hz, 1H), 4.55 (d, J = 15.4 Hz, 1H), 4.32 (t, J = 11.2 |
| | Hz, 1H), 4.27 - 4.21 (m, 1H), 4.16 (d, J = 15.4 Hz, 1H), 3.94 - |
| | 3.84 (m, 1H), 3.03 - 2.86 (m, 7H), 2.23 - 1.83 (m, 7H), 1.38 (d, |
| | J = 14.7 Hz, 1H), 0.52 (s, 9H). |
| 1651 | ¹H NMR (400 MHZ, DMSO-d6) 8 8.83 (s, 1H), 7.96 (d, J = 6.8 |
| | Hz, 1H), 7.69 (d, J = 6.7 Hz, 2H), 7.26 (t, J = 7.6 Hz, 1H), 7.12 |
| | (d, J = 7.6 Hz, 2H), 6.44 (s, 1H), 5.43 (d, J = 6.6 Hz, 1H), 4.84 |
| | (d, J = 16.5 Hz, 1H), 4.41 (d, J = 16.5 Hz, 2H), 4.14 (dt, J = |
| | 12.1, 6.7 Hz, 5H), 2.72 (s, 3H), 2.43 (s, 3H), 2.19 - 1.85 (m, |
| | 9H), 1.83 - 1.72 (m, 2H), 1.50 (tq, J = 9.8, 5.6, 4.7 Hz, 2H), |
| | 1.41 (d, J = 15.0 Hz, 1H), 1.23 (s, 1H), 0.56 (s, 9H). |
| 1652 | ¹H NMR (400 MHz, DMSO-d6) 8 13.09 (s, 1H), 9.41 (s, 1H), |
| | 8.74 (s, 1H), 8.47 (s, 1H), 7.95 (s, 1H), 7.68 (s, 2H), 7.25 (d, J = |
| | 7.8 Hz, 1H), 7.13 (s, 2H), 6.43 (s, 1H), 5.44 (d, J = 8.6 Hz, |
| | 1H), 4.93 (d, J = 16.4 Hz, 1H), 4.66 (d, J = 16.5 Hz, 1H), 4.29 |
| | (t, J = 11.1 Hz, 1H), 4.22 - 4.15 (m, 1H), 4.10 - 4.00 (m, 2H), |
| | 2.61 (t, J = 8.0 Hz, 2H), 2.19 - 2.06 (m, 4H), 1.93 (s, 4H), 1.74 |
| | (dd, J = 15.2, 8.9 Hz, 1H), 1.45 (d, J = 15.0 Hz, 1H), 0.58 (s, |
| | 9H). |
| 1653 | ¹H NMR (400 MHZ, DMSO-d6) 8 13.11 (s, 1H), 9.17 (s, 1H), |
| | 8.67 (s, 1H), 8.51 (s, 1H), 7.96 (s, 1H), 7.74 - 7.63 (m, 2H), 7.27 |
| | (t, J = 7.6 Hz, 1H), 7.13 (d, J = 7.6 Hz, 2H), 6.45 (s, 1H), 5.52 - |
| | 5.44 (m, 1H), 5.01 (t, J = 9.0 Hz, 1H), 4.88 (d, J = 16.2 Hz, |
| | 1H), 4.68 (d, J = 16.1 Hz, 1H), 4.60 (t, J = 8.2 Hz, 1H), 4.45 |
| | (t, J = 11.1 Hz, 1H), 4.19 (dd, J = 8.4, 3.5 Hz, 1H), 4.06 - 3.99 |
| | (m, 1H), 2.03 (d, J = 71.7 Hz, 6H), 1.74 (dd, J = 15.2, 8.9 Hz, |
| | 1H), 1.51 (d, J = 6.1 Hz, 3H), 1.46 (d, J = 14.9 Hz, 1H), 0.59 |
| | (s, 9H). |
| 1654 | ¹H NMR (400 MHZ, DMSO) 8 13.07 (s, 1H), 8.52 - 8.39 (m, |
| | 1H), 8.15 (d, J = 1.4 Hz, 1H), 7.92 (s, 1H), 7.87 (d, J = 1.5 Hz, |
| | 1H), 7.65 (s, 2H), 7.24 (s, 1H), 7.11 (s, 2H), 6.41 (s, 1H), 5.21 |
| | (d, J = 10.2 Hz, 1H), 4.71 (d, J = 15.0 Hz, 1H), 4.37 (d, J = |
| | 14.9 Hz, 2H), 3.99 (s, 1H), 3.73 (s, 4H), 2.18 - 1.78 (m, 7H), |
| | 1.41 (d, J = 15.0 Hz, 1H), 1.29 (s, 6H), 0.54 (s, 9H). |
| 1655 | ¹H NMR (400 MHZ, DMSO) & 12.82 (s, 1H), 8.51 (s, 1H), 7.92 |
| | (d, J = 6.6 Hz, 1H), 7.78 (d, J = 2.8 Hz, 1H), 7.66 (s, 2H), 7.26 |
| | (d, J = 8.5 Hz, 2H), 7.12 (d, J = 7.6 Hz, 2H), 6.82 (dd, J = 8.4, |
| | 2.8 Hz, 1H), 6.40 (s, 1H), 5.18 (d, J = 10.2 Hz, 1H), 4.81 (d, J = |
| | 15.1 Hz, 1H), 4.36 (d, J = 15.1 Hz, 1H), 4.18 (s, 1H), 3.98 |
| | (s, 1H), 3.58 (s, 4H), 1.99 (s, 6H), 1.85 (dd, J = 15.1, 8.9 Hz, |
| | 1H), 1.38 (d, J = 15.0 Hz, 1H), 1.28 (s, 6H), 0.54 (s, 9H). |
| 1657 | ¹H NMR (400 MHZ, DMSO-d6) & 12.80 (s, 1H), 8.66 (s, 1H), |
| | 8.00 - 7.91 (m, 1H), 7.88 (d, J = 1.7 Hz, 2H), 7.85 (d, J = 4.8 |
| | Hz, 1H), 7.68 (d, J = 5.9 Hz, 2H), 7.26 (t, J = 7.6 Hz, 1H), 7.13 |
| | (d, J = 7.7 Hz, 2H), 6.43 (s, 1H), 5.43 (dd, J = 10.9, 4.3 Hz, |
| | 1H), 4.74 (d, J = 15.8 Hz, 1H), 4.43 (d, J = 15.8 Hz, 1H), 4.39 - |
| | 4.25 (m, 2H), 4.06 - 3.96 (m, 3H), 3.61 (s, 1H), 2.54 (d, J = |
| | 4.5 Hz, 3H), 2.24 - 1.85 (m, 11H), 1.80 - 1.70 (m, 1H), 1.43 (d, |
| | J = 14.9 Hz, 1H), 0.57 (s, 9H). |
| 1658 | ¹H NMR (400 MHZ, DMSO-d6) 8 13.05 (s, 1H), 8.66 (s, 1H), |
| | 7.94 (dd, J = 5.4, 2.8 Hz, 1H), 7.86 (d, J = 2.7 Hz, 1H), 7.78 |
| | (d, J = 3.2 Hz, 1H), 7.68 (d, J = 6.0 Hz, 2H), 7.26 (t, J = 7.6 |
| | Hz, 1H), 7.13 (d, J = 7.7 Hz, 2H), 6.42 (s, 1H), 5.42 (dd, J = |
| | 10.9, 4.3 Hz, 1H), 4.72 (d, J = 15.8 Hz, 1H), 4.44 (d, J = 15.4 |
| | Hz, 2H), 4.28 (t, J = 11.2 Hz, 1H), 4.03 (td, J = 11.5, 4.0 Hz, |
| | 2H), 3.65 (s, 1H), 2.90 (s, 3H), 2.77 (s, 3H), 2.75 - 2.68 (m, |
| | 1H), 2.49 - 2.42 (m, 1H), 2.24 - 1.86 (m, 9H), 1.84 (d, J = 5.8 |
| | Hz, 1H), 1.74 (dd, J = 15.2, 8.9 Hz, 1H), 1.42 (d, J = 14.9 Hz, |
| | 1H), 0.56 (s, 9H). |
| 1659 | ¹H NMR (400 MHZ, DMSO-d6) 8 13.11 (s, 1H), 8.71 (s, 1H), |
| | 8.00 - 7.93 (m, 1H), 7.89 (s, 1H), 7.75 (s, 1H), 7.74 - 7.61 (m, |
| | 3H), 7.28 (t, J = 7.6 Hz, 1H), 7.14 (d, J = 7.7 Hz, 2H), 6.46 (s, |
| | 1H), 5.49 (dd, J = 10.8, 4.2 Hz, 1H), 4.69 (d, J = 15.9 Hz, 1H), |
| | 4.57 (d, J = 8.2 Hz, 1H), 4.44 (d, J = 16.0 Hz, 1H), 4.25 (t, J = |
| | 11.2 Hz, 1H), 4.01 (t, J = 10.1, 7.0 Hz, 1H), 2.27 - 2.19 (m, |
| | 1H), 2.18 - 1.88 (m, 9H), 1.71 (dd, J = 15.2, 8.9 Hz, 1H), 1.41 |
| | (d, J = 15.0 Hz, 1H), 0.57 (s, 9H). Protons from -NMe and two |
| | methylenes presumed under water peak. |
| 1660 | ¹H NMR (400 MHZ, DMSO-d6) 8 12.91 (s, 1H), 8.62 (s, 1H), |
| | 7.94 (d, J = 7.0 Hz, 1H), 7.86 (s, 1H), 7.68 (q, J = 7.7 Hz, 3H), |
| | 7.27 (t, J = 7.6 Hz, 1H), 7.14 (d, J = 7.7 Hz, 2H), 6.48 (s, 1H), |
| | 5.42 (dd, J = 11.0, 4.2 Hz, 1H), 5.15 - 4.99 (m, 1H), 4.64 (d, J = |
| | 15.6 Hz, 1H), 4.39 (d, J = 15.6 Hz, 1H), 4.22 (t, J = 11.2 Hz, |
| | 1H), 4.02 (t, J = 11.9 Hz, 1H), 3.07 (s, 3H), 2.83 (s, 3H), 2.37 - |
| | 2.27 (m, 1H), 2.24 - 1.97 (m, 7H), 1.96 - 1.83 (m, 2H), 1.79 - |
| | 1.70 (m, 1H), 1.42 (d, J = 15.0 Hz, 1H), 0.57 (s, 9H). Two |
| | methylene protons presumably under water peak. |
| 1667 | ¹H NMR (400 MHZ, DMSO-d6) & 12.89 (s, 1H), 8.65 (s, 1H), |
| | 8.04 (s, 1H), 7.95 (d, J = 6.7 Hz, 1H), 7.89 (s, 1H), 7.68 (d, J = |
| | 6.5 Hz, 2H), 7.26 (t, J = 7.6 Hz, 1H), 7.13 (d, J = 7.6 Hz, 2H), |
| | 6.44 (s, 1H), 5.39 (dd, J = 10.7, 4.3 Hz, 1H), 4.72 (d, J = 15.8 |
| | Hz, 1H), 4.45 (d, J = 15.9 Hz, 1H), 4.31 (t, J = 11.2 Hz, 1H), |
| | 4.18 (dh, J = 16.7, 5.4 Hz, 2H), 4.10 - 4.00 (m, 1H), 3.95 (dt, J = |
| | 15.0, 5.4 Hz, 1H), 3.79 (dt, J = 14.9, 5.6 Hz, 1H), 3.14 (s, |
| | 3H), 2.73 (s, 3H), 2.68 (d, J = 14.2 Hz, 3H), 2.04 (d, J = 26.7 |
| | Hz, 6H), 1.75 (dd, J = 15.1, 9.1 Hz, 1H), 1.42 (d, J = 15.0 Hz, |
| | 1H), 0.56 (s, 9H). |
| 1668 | ¹H NMR (400 MHZ, Chloroform-d) 8 8.83 (t, J = 1.8 Hz, 1H), |
| | 8.03 (d, J = 7.9 Hz, 1H), 7.95 - 7.82 (m, 3H), 7.63 (t, J = 7.8 |
| | Hz, 1H), 7.22 (t, J = 7.6 Hz, 1H), 7.07 (d, J = 7.6 Hz, 2H), 6.25 |
| | (s, 1H), 5.41 (dd, J = 11.1, 4.3 Hz, 1H), 5.10 (d, J = 14.8 Hz, |
| | 1H), 4.54 (p, J = 8.3 Hz, 1H), 4.50 - 4.37 (m, 1H), 4.06 (t, J = |
| | 11.2 Hz, 1H), 3.95 (d, J = 15.2 Hz, 1H), 3.16 (s, 3H), 2.35 - |
| | 2.25 (m, 2H), 2.22 (q, J = 9.4 Hz, 2H), 2.03 (s, 6H), 1.80 - 1.69 |
| | (m, 3H), 1.40 (dd, J = 15.1, 10.0 Hz, 1H), 0.45 (s, 3H), 0.32 |
| | (dd, J = 9.5, 4.9 Hz, 1H), 0.23 (dt, J = 9.7, 4.9 Hz, 1H), 0.11 |
| | (dt, J = 9.3, 4.9 Hz, 1H), -0.01 - - 0.04 (overlapped with TMS, |
| | m, 1H). |
| 1669 | ¹H NMR (400 MHZ, Chloroform-d) & 8.76 (t, J = 1.8 Hz, 1H), |
| | 8.00 (d, J = 7.9 Hz, 1H), 7.89 (d, J = 3.5 Hz, 2H), 7.85 (d, J = |
| | 7.6 Hz, 1H), 7.63 (t, J = 7.8 Hz, 1H), 7.22 (t, J = 7.6 Hz, 1H), |
| | 7.07 (d, J = 7.6 Hz, 2H), 6.24 (s, 1H), 5.42 - 5.32 (m, 1H), 5.17 |
| | (d, J = 15.6 Hz, 1H), 4.54 (p, J = 8.4 Hz, 1H), 4.13 - 3.95 (m, |
| | 3H), 3.14 (s, 3H), 2.35 - 2.25 (m, 2H), 2.25 - 2.08 (m, 3H), 2.04 |
| | (s, 6H), 2.01 - 1.93 (m, 1H), 1.89 - 1.78 (m, 2H), 1.78 - 1.70 |
| | (m, 2H), 1.70 - 1.53 (m, 4H), 1.04 (p, J = 9.2 Hz, 1H). |
| 1670 | ¹H NMR (400 MHZ, Chloroform-d) 8 8.77 (s, 1H), 8.00 (d, J = |
| | 7.8 Hz, 1H), 7.95 - 7.88 (m, 2H), 7.85 (d, J = 7.5 Hz, 1H), 7.64 |
| | (t, J = 7.8 Hz, 1H), 7.21 (t, J = 7.6 Hz, 1H), 7.06 (d, J = 7.6 |
| | Hz, 2H), 6.18 (s, 1H), 5.38 (dd, J = 11.1, 4.0 Hz, 1H), 5.25 (d, |
| | J = 15.4 Hz, 1H), 4.55 (p, J = 8.6 Hz, 1H), 4.30 - 4.16 (m, 1H), |
| | 4.11 - 3.96 (m, 2H), 3.15 (s, 3H), 2.36 - 2.27 (m, 2H), 2.22 (q, |
| | J = 9.6 Hz, 2H), 2.03 (s, 6H), 1.93 - 1.80 (m, 1H), 1.79 - 1.65 |
| | (m, 2H), 1.31 - 1.24 (m, 1H), 0.56 - 0.41 (m, 2H), 0.26 (tt, J = |
| | 8.8, 4.9 Hz, 1H), 0.08 (dd, J = 9.2, 5.5 Hz, 1H), -0.40 (dq, J = |
| | 9.7, 4.9 Hz, 1H). |
| 1671 | ¹H NMR (400 MHZ, Chloroform-d) 8 8.81 (s, 1H), 8.04 (d, J = |
| | 7.9 Hz, 1H), 7.90 (d, J = 3.6 Hz, 2H), 7.86 (d, J = 7.9 Hz, 1H), |
| | 7.64 (t, J = 7.8 Hz, 1H), 7.21 (t, J = 7.6 Hz, 1H), 7.06 (d, J = |
| | 7.6 Hz, 2H), 6.22 (s, 1H), 5.42 (dd, J = 11.0, 4.0 Hz, 1H), 5.16 |
| | (d, J = 15.5 Hz, 1H), 4.56 (p, J = 8.4 Hz, 1H), 4.23 - 4.12 (m, |
| | 1H), 4.12 - 3.98 (m, 2H), 3.14 (s, 3H), 2.34 - 2.26 (m, 2H), 2.25 - |
| | 2.13 (m, 2H), 2.02 (s, 6H), 1.78 - 1.65 (m, 3H), 1.52 - 1.43 |
| | (m, 1H), 1.40 - 1.29 (m, 1H), 0.84 (d, J = 6.5 Hz, 3H), 0.39 (d, |
| | J = 6.3 Hz, 3H). |
| 1672 | ¹H NMR (400 MHZ, DMSO-d6) 8 8.68 (d, J = 2.1 Hz, 1H), 8.00 |
| | (s, 1H), 7.96 (dt, J = 6.9, 2.0 Hz, 1H), 7.85 (d, J = 1.9 Hz, 1H), |
| | 7.69 (d, J = 6.7 Hz, 2H), 7.26 (t, J = 7.6 Hz, 1H), 7.13 (d, J = |
| | 7.6 Hz, 2H), 6.45 (s, 1H), 5.37 (dd, J = 10.7, 4.4 Hz, 1H), 4.72 |
| | (d, J = 15.8 Hz, 2H), 4.47 (d, J = 16.0 Hz, 1H), 4.31 (t, J = 11.1 |
| | Hz, 1H), 4.07 (dd, J = 20.5, 4.2 Hz, 1H), 2.87 (s, 3H), 2.01 (s, |
| | 6H), 1.75 (dd, J = 15.3, 9.0 Hz, 1H), 1.62 - 1.48 (m, 2H), 1.41 |
| | (d, J = 15.0 Hz, 1H), 1.16 (d, J = 6.6 Hz, 4H), 0.63 (t, J = 7.3 |
| | Hz, 3H), 0.55 (s, 9H). |
| 1673 | ¹H NMR (400 MHZ, DMSO-d6) 8 8.66 (d, J = 2.1 Hz, 1H), |
| | 8.04 - 7.91 (m, 2H), 7.84 (s, 1H), 7.68 (d, J = 7.4 Hz, 2H), 7.25 |
| | (q, J = 6.7, 5.8 Hz, 1H), 7.13 (d, J = 7.7 Hz, 2H), 6.45 (s, 1H), |
| | 5.40 (dd, J = 10.8, 4.4 Hz, 1H), 4.76 - 4.63 (m, 2H), 4.47 (d, J = |
| | 15.9 Hz, 1H), 4.33 (t, J = 11.1 Hz, 1H), 4.10 - 4.00 (m, 1H), |
| | 3.17 (s, 2H), 2.86 (s, 3H), 2.00 (s, 6H), 1.74 (dd, J = 15.3, 9.0 |
| | Hz, 1H), 1.59 (p, J = 7.4 Hz, 2H), 1.41 (d, J = 14.8 Hz, 1H), |
| | 1.07 (d, J = 6.6 Hz, 2H), 0.79 (t, J = 7.3 Hz, 3H), 0.55 (s, 9H). |
| 1674 | ¹H NMR (400 MHZ, DMSO-d6) 8 13.11 (s, 1H), 9.36 (s, 1H), |
| | 8.68 (s, 1H), 8.48 (s, 1H), 7.96 (s, 1H), 7.68 (s, 2H), 7.27 (t, J = |
| | 7.7 Hz, 1H), 7.13 (d, J = 7.7 Hz, 2H), 6.45 (s, 1H), 5.48 (dd, |
| | J = 10.9, 4.3 Hz, 1H), 4.94 - 4.81 (m, 2H), 4.69 (d, J = 16.2 Hz, |
| | 1H), 4.43 (t, J = 11.1 Hz, 1H), 4.08 - 4.00 (m, 1H), 2.80 (dt, J = |
| | 17.2, 9.5 Hz, 1H), 2.44 (dd, J = 9.5, 3.2 Hz, 1H), 2.37 - 2.26 |
| | (m, 1H), 1.93 (s, 6H), 1.81 - 1.70 (m, 2H), 1.50 - 1.38 (m, 4H), |
| | 0.58 (s, 9H). |
| 1675 | ¹H NMR (400 MHZ, DMSO-d6) 8 13.13 (s, 1H), 9.23 (s, 1H), |
| | 8.66 (s, 1H), 8.52 (s, 1H), 7.95 (s, 1H), 7.67 (s, 2H), 7.25 (d, J = |
| | 7.8 Hz, 1H), 7.18 - 7.07 (m, 2H), 6.46 (s, 1H), 5.48 (d, J = |
| | 9.3 Hz, 1H), 4.96 (dt, J = 9.4, 3.7 Hz, 1H), 4.86 (d, J = 16.1 |
| | Hz, 1H), 4.68 (d, J = 16.0 Hz, 1H), 4.40 (t, J = 11.1 Hz, 1H), |
| | 3.99 (d, J = 9.0 Hz, 1H), 2.67 - 2.56 (m, 1H), 2.37 (d, J = 3.8 |
| | Hz, 1H), 2.20 (dd, J = 15.9, 7.0 Hz, 2H), 2.07 (s, 2H), 2.04 - |
| | 1.85 (m, 5H), 1.72 (dd, J = 15.2, 9.0 Hz, 1H), 1.44 (d, J = 14.8 |
| | Hz, 1H), 0.88 (d, J = 6.8 Hz, 3H), 0.72 (d, J = 6.8 Hz, 3H), 0.59 |
| | (s, 9H). |
| 1676 | ¹H NMR (400 MHZ, Chloroform-d) 8 8.90 (d, J = 2.5 Hz, 1H), |
| | 8.09 (d, J = 7.8 Hz, 1H), 7.92 (d, J = 3.8 Hz, 1H), 7.85 (d, J = |
| | 6.8 Hz, 2H), 7.69 - 7.60 (m, 1H), 7.20 (t, J = 7.6 Hz, 1H), 7.06 |
| | (d, J = 7.6 Hz, 2H), 6.23 (s, 1H), 5.39 (dd, J = 11.3, 4.3 Hz, |
| | 1H), 5.18 (d, J = 15.6 Hz, 1H), 4.54 (q, J = 8.8 Hz, 1H), 4.23 |
| | (s, 1H), 4.10 (d, J = 15.7 Hz, 1H), 3.95 (t, J = 11.4 Hz, 1H), |
| | 3.80 (d, J = 9.0 Hz, 1H), 3.74 (d, J = 10.1 Hz, 1H), 3.45 - 3.37 |
| | (m, 2H), 3.25 (d, J = 7.2 Hz, 1H), 3.21 (s, 3H), 2.73 (dt, J = |
| | 11.2, 5.4 Hz, 1H), 2.61 - 2.51 (m, 1H), 2.34 - 2.19 (m, 2H), 1.72 |
| | (dd, J = 15.2, 8.8 Hz, 1H), 1.51 (s, 1H), 1.26 (s, 5H), 0.62 (s, |
| | 9H). |
| 1677 | ¹H NMR (400 MHZ, Chloroform-d) 8 8.86 (t, J = 1.8 Hz, 1H), |
| | 8.10 (d, J = 7.8 Hz, 1H), 7.96 (s, 1H), 7.91 - 7.84 (m, 2H), 7.67 |
| | (t, J = 7.8 Hz, 1H), 7.21 (t, J = 7.6 Hz, 1H), 7.07 (d, J = 7.6 |
| | Hz, 2H), 6.27 (s, 1H), 5.40 (dd, J = 11.2, 4.3 Hz, 1H), 5.09 (d, |
| | J = 15.5 Hz, 1H), 4.56 (q, J = 8.9 Hz, 1H), 4.29 - 4.22 (m, 1H), |
| | 4.13 - 4.02 (m, 2H), 3.86 (dd, J = 9.6, 5.8 Hz, 2H), 3.52 - 3.46 |
| | (m, 2H), 3.35 (d, J = 7.1 Hz, 1H), 3.21 (s, 3H), 2.88 - 2.82 (m, |
| | 1H), 2.58 - 2.49 (m, 1H), 2.27 (dt, J = 12.5, 8.2 Hz, 1H), 2.02 |
| | (s, 4H), 1.73 (dd, J = 15.2, 8.8 Hz, 2H), 1.26 (s, 2H), 0.61 (s, |
| | 9H). |
| 1678 | ¹H NMR (400 MHZ, Chloroform-d) 8 8.85 (s, 1H), 8.09 (d, J = |
| | 8.0 Hz, 1H), 7.87 (s, 1H), 7.84 (dd, J = 82.3, 8.1 Hz, 1H), 7.63 |
| | (t, J = 7.8 Hz, 1H), 7.20 (t, J = 7.6 Hz, 1H), 7.06 (d, J = 7.6 |
| | Hz, 2H), 6.22 (s, 1H), 5.45 (dd, J = 11.4, 4.3 Hz, 1H), 5.17 (d, |
| | J = 15.3 Hz, 1H), 4.70 (p, J = 8.4 Hz, 1H), 4.19 - 4.10 (m, 1H), |
| | 4.07 (d, J = 15.3 Hz, 1H), 3.92 (t, J = 11.5 Hz, 1H), 3.49 (s, |
| | 2H), 3.05 (s, 3H), 2.60 (s, 3H), 2.31 - 2.22 (m, 2H), 2.22 - 2.14 |
| | (m, 2H), 1.84 - 1.77 (m, 1H), 1.77 - 1.61 (m, 4H), 1.53 (d, J = |
| | 14.3 Hz, 3H), 0.64 (s, 9H). |
| 1679 | ¹H NMR (400 MHZ, DMSO-d6) 8 12.98 (s, 1H), 8.71 (s, 1H), |
| | 8.17 (s, 1H), 8.03 - 7.87 (m, 2H), 7.70 (s, 2H), 7.25 (t, J = 7.7 |
| | Hz, 1H), 7.12 (d, J = 7.7 Hz, 2H), 6.47 (s, 1H), 5.22 (d, J = 9.2 |
| | Hz, 1H), 4.95 (q, J = 7.9 Hz, 1H), 4.84 (d, J = 16.4 Hz, 1H), |
| | 4.50 (d, J = 16.4 Hz, 1H), 4.13 (t, J = 15.8 Hz, 3H), 3.83 (t, J = |
| | 8.1 Hz, 1H), 3.72 (dd, J = 8.5, 5.0 Hz, 1H), 3.54 (d, J = 8.4 |
| | Hz, 1H), 3.39 - 3.38 (m, 1H), 2.94 (t, J = 11.4 Hz, 1H), 2.32 - |
| | 2.22 (m, 1H), 2.21 - 1.83 (m, 7H), 1.83 - 1.70 (m, 2H), 1.53 - |
| | 1.37 (m, 3H), 0.55 (s, 9H). |
| 1680 | ¹H NMR (400 MHZ, DMSO-d6) 8 13.09 (s, 1H), 8.61 (s, 1H), |
| | 8.15 (s, 1H), 8.04 - 7.87 (m, 2H), 7.67 (s, 2H), 7.25 (t, J = 7.9 |
| | Hz, 1H), 7.12 (d, J = 7.5 Hz, 2H), 6.54 - 6.37 (m, 1H), 5.42 (d, |
| | J = 9.6 Hz, 1H), 5.04 (q, J = 8.0 Hz, 1H), 4.67 (d, J = 15.7 Hz, |
| | 1H), 4.47 (d, J = 15.7 Hz, 1H), 4.44 - 4.32 (m, 1H), 4.11 (d, J = |
| | 12.8 Hz, 1H), 3.99 (t, J = 8.1 Hz, 2H), 3.88 (dd, J = 8.4, 5.0 |
| | Hz, 1H), 3.59 (d, J = 8.3 Hz, 1H), 3.48 (t, J = 8.4 Hz, 1H), 2.93 |
| | (t, J = 11.1 Hz, 1H), 2.36 - 2.28 (m, 1H), 2.18 - 1.83 (m, 7H), |
| | 1.80 - 1.70 (m, 2H), 1.59 - 1.43 (m, 3H), 0.56 (s, 9H). |
| 1682 | ¹H NMR (400 MHZ, Chloroform-d) 8 8.74 (d, J = 1.9 Hz, 1H), |
| | 8.03 (d, J = 7.9 Hz, 1H), 7.88 (s, 1H), 7.87 - 7.81 (m, 2H), 7.66 |
| | (t, J = 7.7 Hz, 1H), 7.22 (t, J = 7.6 Hz, 1H), 7.07 (d, J = 7.6 |
| | Hz, 2H), 6.23 (s, 1H), 5.39 (d, J = 8.5 Hz, 1H), 5.15 (d, J = |
| | 15.1 Hz, 1H), 4.51 (t, J = 8.6 Hz, 1H), 4.06 (s, 3H), 3.18 (s, |
| | 3H), 2.31 (q, J = 8.7, 7.7 Hz, 2H), 2.22 (q, J = 9.5 Hz, 2H), |
| | 2.05 (s, 6H), 1.98 - 1.83 (m, 4H), 1.81 - 1.71 (m, 5H), 1.70 - |
| | 1.59 (m, 4H), 1.52 (dd, J = 10.9, 7.7 Hz, 1H), 0.95 (dd, J = |
| | 10.9, 7.8 Hz, 1H). |
| 1683 | ¹H NMR (400 MHZ, CDC13) 8 8.82 (t, J = 1.6 Hz, 1H), 8.01 (d, |
| | J = 7.8 Hz, 1H), 7.90 (s, 2H), 7.82 (d, J = 7.5 Hz, 1H), 7.62 (t, |
| | J = 7.7 Hz, 1H), 7.22 (t, J = 7.6 Hz, 1H), 7.07 (d, J = 7.6 Hz, |
| | 2H), 6.25 (s, 1H), 5.41 (d, J = 9.2 Hz, 1H), 5.13 (d, J = 14.2 |
| | Hz, 1H), 4.60 - 4.46 (m, 1H), 4.29 - 4.18 (m, 1H), 4.18 - 3.99 |
| | (m, 2H), 3.16 (s, 3H), 2.34 - 2.26 (m, 2H), 2.25 - 2.17 (m, 2H), |
| | 2.02 (s, 6H), 1.93 - 1.81 (m, 2H), 1.77 - 1.70 (m, 3H), 1.66 (d, |
| | J = 14.7 Hz, 2H), 1.58 - 1.47 (m, 1H), 1.38 (t, J = 7.6 Hz, 2H), |
| | 0.65 (s, 3H). |
| 1684 | ¹H NMR (400 MHZ, CDC13) 8 8.75 (t, J = 1.8 Hz, 1H), 7.93 (d, |
| | J = 7.9 Hz, 1H), 7.88 (d, J = 8.1 Hz, 2H), 7.81 (d, J = 7.6 Hz, |
| | 1H), 7.62 (t, J = 7.8 Hz, 1H), 7.23 (t, J = 7.6 Hz, 1H), 7.06 (d, |
| | J = 7.6 Hz, 2H), 6.21 (s, 1H), 5.45 - 5.35 (m, 1H), 5.16 (d, J = |
| | 15.3 Hz, 1H), 4.53 (p, J = 8.4 Hz, 1H), 4.22 - 3.97 (m, 3H), |
| | 3.17 (s, 3H), 2.37 - 2.27 (m, 2H), 2.26 - 2.16 (m, 2H), 2.04 (s, |
| | 6H), 1.86 - 1.65 (m, 4H), 1.12 - 0.97 (m, 2H), 0.48 - 0.35 (m, |
| | 1H), 0.35 - 0.21 (m, 2H), -0.07 - - 0.19 (m, 2H). |
| 1319 | ¹H NMR (400 MHZ, CDC13) 8 8.76 (t, J = 1.8 Hz, 1H), 8.07 (d, |
| | J = 7.8 Hz, 1H), 7.90 (s, 1H), 7.88 - 7.81 (m, 2H), 7.68 (t, J = |
| | 7.8 Hz, 1H), 7.22 (t, J = 7.6 Hz, 1H), 7.06 (d, J = 7.6 Hz, 2H), |
| | 6.24 (s, 1H), 5.42 (d, J = 7.0 Hz, 1H), 5.15 (d, J = 15.7 Hz, |
| | 1H), 4.58 - 4.45 (m, 1H), 4.14 - 4.01 (m, 3H), 3.19 (s, 3H), 2.40 - |
| | 2.28 (m, 2H), 2.28 - 2.18 (m, 2H), 2.04 (s, 6H), 1.82 - 1.71 |
| | (m, 2H), 1.68 - 1.56 (m, 2H), 1.20 - 1.07 (m, 1H), 0.81 (s, 9H), |
| | 0.80 - 0.74 (m, 1H). |
| 1685 | ¹H NMR (400 MHZ, CDC13) 8 8.74 (d, J = 1.7 Hz, 1H), 7.96 |
| | (d, J = 7.9 Hz, 1H), 7.89 (d, J = 5.4 Hz, 2H), 7.82 (d, J = 7.6 |
| | Hz, 1H), 7.63 (t, J = 7.7 Hz, 1H), 7.22 (t, J = 7.6 Hz, 1H), 7.05 |
| | (d, J = 7.6 Hz, 2H), 6.17 (s, 1H), 5.43 - 5.35 (m, 1H), 5.19 (d, |
| | J = 15.2 Hz, 1H), 4.53 (p, J = 8.4 Hz, 1H), 4.14 - 3.94 (m, 3H), |
| | 3.17 (s, 3H), 2.40 - 2.26 (m, 2H), 2.26 - 2.14 (m, 2H), 2.02 (s, |
| | 6H), 1.80 - 1.71 (m, 2H), 1.70 - 1.55 (m, 2H), 1.39 - 1.26 (m, |
| | 1H), 1.17 - 1.04 (m, 1H), 0.95 - 0.86 (m, 1H), 0.80 (d, J = 6.5 |
| | Hz, 3H), 0.78 (d, J = 6.5 Hz, 3H). |
| 1688 | ¹H NMR (400 MHZ, DMSO-d6) 8 13.13 (s, 1H), 8.60 (s, 1H), |
| | 8.00 (s, 1H), 7.92 (d, J = 7.6 Hz, 1H), 7.86 (s, 1H), 7.80 - 7.56 |
| | (m, 2H), 7.26 (t, J = 7.6 Hz, 1H), 7.13 (d, J = 7.7 Hz, 2H), 6.44 |
| | (s, 1H), 5.36 (dd, J = 10.7, 4.3 Hz, 1H), 4.79 (p, J = 8.5 Hz, |
| | 1H), 4.69 (d, J = 16.0 Hz, 1H), 4.45 (dq, J = 11.0, 4.1 Hz, 1H), |
| | 4.35 (s, 1H), 4.32 - 4.26 (m, 1H), 3.07 (s, 3H), 2.29 - 1.79 (m, |
| | 11H), 1.74 - 1.58 (m, 3H), 0.90 - 0.85 (m, 1H), 0.82 - 0.75 (m, |
| | 1H), 0.74 - 0.64 (m, 1H), 0.64 - 0.54 (m, 1H). |
| 1689 | ¹H NMR (400 MHZ, DMSO-d6) & 12.99 (s, 1H), 8.68 (s, 1H), |
| | 7.99 (s, 1H), 7.96 (t, J = 5.0 Hz, 1H), 7.88 (s, 1H), 7.70 (d, J = |
| | 4.6 Hz, 2H), 7.26 (t, J = 7.6 Hz, 1H), 7.13 (d, J = 7.7 Hz, 2H), |
| | 6.46 (s, 1H), 5.43 (dd, J = 10.8, 4.3 Hz, 1H), 4.85 - 4.70 (m, |
| | 2H), 4.54 (d, J = 15.9 Hz, 1H), 4.37 (t, J = 11.1 Hz, 1H), 4.26 - |
| | 4.16 (m, 1H), 3.07 (s, 3H), 2.25 - 1.86 (m, 11H), 1.80 (d, J = |
| | 15.7 Hz, 1H), 1.70 - 1.60 (m, 2H), 0.93 (s, 3H), 0.69 (s, 3H). |
| 1690 | ¹H NMR (400 MHZ, DMSO-d6) 8 12.95 (s, 1H), 8.61 (s, 1H), |
| | 7.99 (s, 1H), 7.92 (d, J = 5.8 Hz, 1H), 7.87 (s, 1H), 7.73 - 7.60 |
| | (m, 2H), 7.26 (t, J = 7.6 Hz, 1H), 7.13 (d, J = 7.6 Hz, 2H), 6.38 |
| | (s, 1H), 5.45 (dd, J = 11.5, 3.9 Hz, 1H), 4.83 (d, J = 15.8 Hz, |
| | 1H), 4.76 (p, J = 8.4 Hz, 1H), 4.59 (t, J = 11.4 Hz, 1H), 4.35 |
| | (d, J = 15.8 Hz, 1H), 3.69 (ddd, J = 11.8, 8.9, 3.8 Hz, 1H), 3.06 |
| | (s, 3H), 2.18 (td, J = 9.2, 4.4 Hz, 5H), 2.11 - 1.91 (m, 5H), 1.63 |
| | (ddd, J = 9.8, 7.8, 3.8 Hz, 2H), 1.28 - 1.22 (m, 1H), 0.79 (dd, J |
| | = 6.9 Hz, 6H). |
| 1693 | ¹H NMR (400 MHZ, Chloroform-d) 8 8.84 (s, 1H), 8.04 (dd, J = |
| | 7.9, 1.4 Hz, 1H), 7.94 (s, 1H), 7.90 (s, 1H), 7.87 (d, J = 7.7, |
| | 1.4 Hz, 1H), 7.64 (t, J = 7.8 Hz, 1H), 7.21 (t, J = 7.6 Hz, 1H), |
| | 7.07 (d, J = 7.6 Hz, 2H), 6.27 (s, 1H), 5.47 (dd, J = 11.2, 4.2 |
| | Hz, 1H), 5.05 (d, J = 15.4 Hz, 1H), 4.28 - 4.20 (m, 1H), 4.14 - |
| | 4.05 (m, 2H), 3.72 - 3.60 (m, 2H), 3.38 (t, J = 5.9 Hz, 2H), 3.28 |
| | (s, 3H), 3.18 (s, 3H), 2.01 (s, 6H), 1.93 - 1.86 (m, 2H), 1.74 - |
| | 1.66 (m, 1H), 1.55 (d, J = 15.1, 1.7 Hz, 1H), 0.61 (s, 9H). |
| 1694 | ¹H NMR (400 MHZ, Chloroform-d) 8 8.87 (d, J = 1.8 Hz, 1H), |
| | 8.03 (d, J = 7.8, 1.5 Hz, 1H), 7.90 - 7.84 (m, 3H), 7.64 (t, J = |
| | 7.8 Hz, 1H), 7.21 (t, J = 7.6 Hz, 1H), 7.07 (d, J = 7.6 Hz, 2H), |
| | 6.26 (s, 1H), 5.47 (dd, J = 11.2, 4.3 Hz, 1H), 5.05 (d, J = 15.5 |
| | Hz, 1H), 4.30 - 4.20 (m, 1H), 4.14 - 4.03 (m, 2H), 3.73 - 3.53 |
| | (m, 2H), 3.14 (s, 3H), 2.02 (s, 6H), 1.70 (dd, J = 15.1, 8.8 Hz, |
| | 2H), 1.54 (d, J = 15.1, 1.7 Hz, 2H), 1.20 (t, J = 7.1 Hz, 3H), |
| | 0.61 (s, 9H). |
| 1695 | ¹H NMR (400 MHZ, Chloroform-d) 8 8.85 (d, J = 1.8 Hz, 1H), |
| | 8.11 (d, J = 7.9 Hz, 1H), 7.89 (s, 1H), 7.86 (d, J = 7.4 Hz, 2H), |
| | 7.66 (t, J = 7.8 Hz, 1H), 7.37 - 7.31 (m, 1H), 7.26 - 7.21 (m, |
| | 3H), 6.40 (s, 1H), 5.46 (dd, J = 11.1, 4.2 Hz, 1H), 5.07 (d, J = |
| | 15.5 Hz, 1H), 4.58 - 4.46 (m, 1H), 4.26 - 4.18 (m, 1H), 4.16 (d, |
| | J = 15.5 Hz, 1H), 4.03 (t, J = 11.4 Hz, 1H), 3.19 (s, 3H), 2.29 |
| | (s, 6H), 2.26 - 2.19 (m, 2H), 1.80 - 1.74 (m, 2H), 1.73 - 1.65 |
| | (m, 2H), 0.65 (s, 9H). |
| 1696 | ¹H NMR (400 MHZ, Chloroform-d) 8 8.84 (d, J = 1.9 Hz, 1H), |
| | 8.05 (d, J = 7.9, 1.5 Hz, 1H), 7.92 - 7.84 (m, 3H), 7.64 (t, J = |
| | 7.8 Hz, 1H), 7.21 (t, J = 7.6 Hz, 1H), 7.07 (d, J = 7.6 Hz, 2H), |
| | 6.26 (s, 1H), 5.45 (dd, J = 11.2, 4.2 Hz, 1H), 5.07 (d, J = 15.4 |
| | Hz, 1H), 4.28 - 4.19 (m, 1H), 4.14 - 4.04 (m, 2H), 3.60 - 3.45 |
| | (m, 2H), 3.16 (s, 3H), 1.77 - 1.43 (m, 11H), 0.92 (d, J = 6.4 Hz, |
| | 6H), 0.62 (s, 9H). |
| 1697 | ¹H NMR (400 MHZ, Chloroform-d) 8 8.86 (s, 1H), 8.05 (d, J = |
| | 8.1, 1.4 Hz, 1H), 7.90 - 7.83 (m, 3H), 7.64 (t, J = 7.8 Hz, 1H), |
| | 7.21 (t, J = 7.6 Hz, 1H), 7.08 (d, J = 7.6 Hz, 2H), 6.28 (s, 1H), |
| | 5.45 (dd, J = 11.3, 4.4 Hz, 1H), 5.02 (d, J = 15.5 Hz, 1H), 4.33 - |
| | 4.25 (m, 1H), 4.14 - 4.04 (m, 2H), 3.67 - 3.50 (m, 4H), 2.02 |
| | (s, 6H), 1.72 - 1.67 (m, 1H), 1.53 (d, J = 13.6 Hz, 1H), 1.22 (t, |
| | J = 7.1 Hz, 6H), 0.60 (s, 9H). |
| 1320 | ¹H NMR (400 MHZ, Chloroform-d) 8 8.77 (d, J = 1.9 Hz, 1H), |
| | 8.11 (d, J = 7.9 Hz, 1H), 7.93 - 7.87 (m, 3H), 7.69 (t, J = 7.8 |
| | Hz, 1H), 7.21 (t, J = 7.6 Hz, 1H), 7.07 (d, J = 7.6 Hz, 2H), 6.16 |
| | (s, 1H), 5.43 (dd, J = 11.4, 4.0 Hz, 1H), 5.12 (d, J = 15.5 Hz, |
| | 1H), 4.56 (q, J = 8.3 Hz, 1H), 4.17 (t, J = 11.4 Hz, 1H), 4.03 - |
| | 3.91 (m, 2H), 2.62 (q, J = 8.7 Hz, 1H), 2.28 (td, J = 7.6, 3.1 |
| | Hz, 2H), 2.19 (dt, J = 20.1, 10.1 Hz, 3H), 2.05 (s, 6H), 1.99 (t, |
| | J = 6.6 Hz, 3H), 1.73 (dd, J = 7.0, 3.1 Hz, 2H), 1.65 (dd, J = |
| | 8.7, 4.4 Hz, 3H), 1.48 (d, J = 6.4 Hz, 2H), 1.34 - 1.28 (m, 2H), |
| | 1.25 (s, 1H), 1.08 - 1.02 (m, 1H). |
| 1321 | ¹H NMR (400 MHZ, Chloroform-d) 8 8.76 (d, J = 1.8 Hz, 1H), |
| | 8.10 (d, J = 7.9 Hz, 1H), 7.90 (d, J = 5.8 Hz, 3H), 7.68 (t, J = |
| | 7.8 Hz, 1H), 7.21 (t, J = 7.6 Hz, 1H), 7.06 (d, J = 7.6 Hz, 2H), |
| | 6.14 (s, 1H), 5.43 (dd, J = 11.3, 4.0 Hz, 1H), 5.12 (d, J = 15.5 |
| | Hz, 1H), 4.57 (h, J = 8.4, 7.9 Hz, 1H), 4.15 (t, J = 11.4 Hz, 1H), |
| | 4.01 - 3.93 (m, 2H), 2.66 - 2.56 (m, 1H), 2.33 - 2.25 (m, 2H), |
| | 2.20 (q, J = 10.0 Hz, 2H), 2.04 (s, 6H), 1.98 (d, J = 7.7 Hz, |
| | 3H), 1.73 (dt, J = 6.1, 2.9 Hz, 2H), 1.66 (td, J = 9.1, 7.9, 3.2 |
| | Hz, 3H), 1.53 - 1.46 (m, 2H), 1.32 (t, J = 10.2 Hz, 2H), 1.26 (s, |
| | 1H), 1.06 (dd, J = 11.0, 8.5 Hz, 1H), 0.90 - 0.84 (m, 1H). |
| 1698 | ¹H NMR (400 MHZ, Chloroform-d) 8 8.85 (s, 1H), 8.06 (d, 1H), |
| | 7.94 - 7.81 (m, 3H), 7.64 (t, J = 7.8 Hz, 1H), 7.21 (t, J = 7.6 |
| | Hz, 1H), 7.07 (d, J = 7.6 Hz, 2H), 6.26 (s, 1H), 5.46 (dd, J = |
| | 11.1, 4.2 Hz, 1H), 5.06 (d, J = 15.4 Hz, 1H), 4.28 - 4.19 (m, |
| | 1H), 4.14 - 4.05 (m, 2H), 3.56 - 3.48 (m, 2H), 3.17 (s, 3H), 2.02 |
| | (s, 6H), 1.72 - 1.52 (m, 4H), 1.34 (dt, J = 15.1, 7.5 Hz, 2H), |
| | 0.91 (t, J = 7.3 Hz, 3H), 0.62 (s, 9H). |
| 1701 | ¹H NMR (400 MHZ, Chloroform-d) 8 8.81 (t, J = 1.8 Hz, 1H), |
| | 8.01 (d, J = 8.4 Hz, 1H), 7.90 (d, J = 8.7 Hz, 2H), 7.86 (d, J = |
| | 7.6 Hz, 1H), 7.64 (t, J = 7.8 Hz, 1H), 7.22 (t, J = 7.6 Hz, 1H), |
| | 7.08 (d, J = 7.6 Hz, 2H), 6.25 (s, 1H), 5.44 (dd, J = 11.3, 4.5 |
| | Hz, 1H), 5.14 (d, J = 15.5 Hz, 1H), 4.61 - 4.41 (m, 2H), 4.14 - |
| | 4.02 (m, 2H), 3.16 (s, 3H), 2.38 - 2.15 (m, 4H), 2.12 - 2.06 (m, |
| | 1H), 2.03 (s, 6H), 1.88 (dd, J = 22.4, 15.9 Hz, 1H), 1.79 - 1.70 |
| | (m, 2H), 1.21 (d, J = 21.2 Hz, 3H), 0.93 (d, J = 20.9 Hz, 3H). |
| 1702 | ¹H NMR (400 MHZ, CDC13) 8 8.80 (t, J = 1.8 Hz, 1H), 8.02 (d, |
| | J = 7.9 Hz, 1H), 7.86 (s, 2H), 7.85 - 7.79 (m, 1H), 7.65 (t, J = |
| | 7.8 Hz, 1H), 7.22 (t, J = 7.6 Hz, 1H), 7.07 (d, J = 7.6 Hz, 2H), |
| | 6.23 (s, 1H), 5.49 (dd, J = 11.7, 4.2 Hz, 1H), 5.18 (d, J = 15.4 |
| | Hz, 1H), 4.50 (p, J = 8.5 Hz, 1H), 4.38 (t, J = 11.6 Hz, 1H), |
| | 4.23 - 4.12 (m, 1H), 4.09 (d, J = 15.9 Hz, 1H), 3.18 (s, 3H), |
| | 2.35 - 2.26 (m, 2H), 2.26 - 2.15 (m, 2H), 2.05 (s, 6H), 1.89 - |
| | 1.82 (m, 1H), 1.77 - 1.70 (m, 2H), 1.45 - 1.35 (m, 1H), 1.23 - |
| | 1.10 (m, 1H), 0.99 (d, J = 6.8 Hz, 3H), 0.60 (t, J = 7.3 Hz, 3H). |
| 1703 | ¹H NMR (400 MHZ, CDC13) 8 8.79 (t, J = 1.8 Hz, 1H), 8.05 (d, |
| | J = 7.8 Hz, 1H), 7.91 - 7.76 (m, 3H), 7.67 (t, J = 7.8 Hz, 1H), |
| | 7.23 (t, J = 7.6 Hz, 1H), 7.09 (d, J = 7.6 Hz, 2H), 6.26 (s, 1H), |
| | 5.52 (dd, J = 11.4, 4.1 Hz, 1H), 5.16 (d, J = 15.4 Hz, 1H), 4.57 - |
| | 4.43 (m, 1H), 4.39 (t, J = 11.3 Hz, 1H), 4.24 - 4.10 (m, 2H), |
| | 3.23 (s, 3H), 2.39 - 2.28 (m, 2H), 2.27 - 2.18 (m, 2H), 2.07 (s, |
| | 6H), 1.87 - 1.83 (m, 1H), 1.81 - 1.74 (m, 2H), 1.43 - 1.37 (m, |
| | 1H), 1.21 - 1.12 (m, 1H), 1.00 (d, J = 6.8 Hz, 3H), 0.61 (t, J = |
| | 7.4 Hz, 3H). |
| 1704 | ¹H NMR (400 MHZ, Chloroform-d) 8 8.82 (s, 1H), 7.89 - 7.83 |
| | (m, 2H), 7.82 - 7.78 (m, 1H), 7.71 - 7.64 (m, 1H), 7.61 (t, J = |
| | 7.8 Hz, 1H), 7.48 - 7.45 (m, 1H), 7.43 - 7.40 (m, 1H), 7.20 (d, |
| | J = 7.7, 1.5 Hz, 1H), 6.43 (s, 1H), 5.47 (dd, J = 11.1, 4.2 Hz, |
| | 1H), 5.05 (d, J = 15.5 Hz, 1H), 4.49 (s, 1H), 4.28 - 4.17 (m, |
| | 2H), 4.06 (t, J = 11.3 Hz, 1H), 3.23 (s, 3H), 3.14 - 3.03 (m, 2H), |
| | 2.36 - 2.30 (m, 2H), 2.27 - 2.20 (m, 2H), 1.84 - 1.75 (m, 2H), |
| | 1.70 - 1.59 (m, 2H), 1.14 (d, J = 6.8 Hz, 3H), 1.09 (d, J = 6.8 |
| | Hz, 3H), 0.62 (s, 9H). |
| 1705 | ¹H NMR (400 MHZ, Chloroform-d) 8 8.78 (s, 1H), 8.05 (s, 1H), |
| | 7.85 (dt, J = 7.6, 1.4 Hz, 1H), 7.64 (t, J = 7.3 Hz, 1H), 7.21 (t, |
| | J = 7.6 Hz, 1H), 7.06 (d, J = 7.6 Hz, 2H), 6.76 (d, J = 73.2 Hz, |
| | 2H), 6.41 (s, 1H), 6.25 (d, J = 1.4 Hz, 1H), 5.24 (d, J = 66.9 |
| | Hz, 2H), 4.78 (s, 1H), 4.24 (s, 1H), 2.91 (s, 2H), 2.04 - 1.97 (m, |
| | 6H), 1.69 - 1.53 (m, 6H), 1.29 (d, J = 41.6 Hz, 9H), 0.62 (s, |
| | 2H). |
| 1707 | ¹H NMR (400 MHZ, Chloroform-d) 8 8.83 (d, J = 1.8 Hz, 1H), |
| | 8.00 (dt, J = 8.0, 1.5 Hz, 1H), 7.92 (s, 1H), 7.86 (dt, J = 7.7, |
| | 1.4 Hz, 1H), 7.81 (s, 1H), 7.62 (t, J = 7.8 Hz, 1H), 7.21 (t, J = |
| | 7.6 Hz, 1H), 7.07 (d, J = 7.6 Hz, 2H), 6.25 (s, 1H), 5.46 (dd, J = |
| | 11.3, 4.3 Hz, 1H), 5.05 (d, J = 15.5 Hz, 1H), 4.26 - 4.15 (m, |
| | 1H), 4.12 - 3.96 (m, 4H), 3.40 (d, J = 7.2 Hz, 2H), 3.34 (s, 3H), |
| | 3.10 - 3.00 (m, 2H), 2.63 - 2.53 (m, 1H), 2.53 - 2.44 (m, 2H), |
| | 2.12 - 1.89 (m, 8H), 1.69 (dd, J = 15.1, 8.8 Hz, 1H), 1.55 - 1.51 |
| | (m, 1H), 0.61 (s, 9H). Sulfonamide NH not observed |
| 1708 | ¹H NMR (400 MHZ, Chloroform-d) 8 8.89 (t, J = 1.8 Hz, 1H), |
| | 8.06 (dt, J = 8.2, 1.3 Hz, 1H), 7.86 (dt, J = 7.7, 1.4 Hz, 1H), |
| | 7.84 (s, 1H), 7.65 (t, J = 7.8 Hz, 1H), 7.57 (s, 1H), 7.22 (t, J = |
| | 7.6 Hz, 1H), 7.08 (d, J = 7.6 Hz, 2H), 6.26 (s, 1H), 5.46 (dd, J = |
| | 11.4, 4.4 Hz, 1H), 5.06 (d, J = 15.9 Hz, 1H), 4.85 (t, J = 5.2 |
| | Hz, 1H), 4.26 - 4.16 (m, 2H), 4.12 (d, J = 15.9 Hz, 1H), 3.99 (t, |
| | J = 11.5 Hz, 1H), 3.67 (dd, J = 9.0, 3.9 Hz, 1H), 3.17 - 3.08 |
| | (m, 1H), 2.07 (d, J = 15.1 Hz, 2H), 2.02 (s, 6H), 1.96 - 1.80 (m, |
| | 4H), 1.66 (dd, J = 15.1, 8.6 Hz, 1H), 1.58 - 1.51 (m, 2H), 0.61 |
| | (s, 9H). |
| 1709 | ¹H NMR (400 MHZ, Chloroform-d) 8 8.83 (t, J = 1.8 Hz, 1H), |
| | 8.05 (dt, J = 8.1, 1.4 Hz, 1H), 7.87 - 7.82 (m, 2H), 7.65 (t, J = |
| | 7.8 Hz, 1H), 7.56 (s, 1H), 7.22 (t, J = 7.6 Hz, 1H), 7.08 (d, J = |
| | 7.6 Hz, 2H), 6.28 (s, 1H), 5.56 (dd, J = 11.3, 4.2 Hz, 1H), 4.99 |
| | (d, J = 15.6 Hz, 1H), 4.87 (t, J = 5.3 Hz, 1H), 4.22 (dt, J = 16.6, |
| | 8.2 Hz, 2H), 4.13 (d, J = 15.6 Hz, 1H), 4.04 (t, J = 11.4 Hz, |
| | 1H), 3.71 (dd, J = 9.1, 4.0 Hz, 1H), 3.21 - 3.13 (m, 1H), 2.13 |
| | (dd, J = 14.4, 4.6 Hz, 2H), 2.04 (s, 6H), 1.98 - 1.83 (m, 4H), |
| | 1.65 (dd, J = 15.1, 8.5 Hz, 1H), 1.59 - 1.53 (m, 2H), 0.61 (s, |
| | 9H). |
| 1710 | ¹H NMR (400 MHZ, CDC13) 8 8.91 - 8.55 (m, 1H), 7.99 (d, J = |
| | 7.9 Hz, 1H), 7.92 (s, 1H), 7.87 - 7.79 (m, 2H), 7.64 (t, J = 7.8 |
| | Hz, 1H), 7.23 (t, J = 7.6 Hz, 1H), 7.07 (d, J = 7.6 Hz, 2H), 6.20 |
| | (s, 1H), 5.43 (dd, J = 11.0, 4.0 Hz, 1H), 5.20 (d, J = 15.4 Hz, |
| | 1H), 4.24 (t, J = 11.0 Hz, 1H), 4.19 - 4.02 (m, 2H), 3.40 (d, J = |
| | 7.4 Hz, 2H), 3.26 (s, 3H), 2.13 - 2.06 (m, 1H), 2.05 (s, 6H), 1.92 - |
| | 1.76 (m, 1H), 1.35 - 1.26 (m, 1H), 0.94 (d, J = 6.5 Hz, 3H), |
| | 0.92 (d, J = 6.5 Hz, 3H), 0.59 - 0.40 (m, 2H), 0.34 - 0.21 (m, |
| | 1H), 0.13 - 0.04 (m, 1H), -0.32 - - 0.47 (m, 1H). |
| 1711 | ¹H NMR (400 MHz, CDC13) 8 8.81 (t, J = 1.7 Hz, 1H), 8.03 |
| | (dt, J = 7.9, 1.4 Hz, 1H), 7.90 (s, 1H), 7.90 - 7.85 (m, 2H), 7.63 |
| | (t, J = 7.8 Hz, 1H), 7.21 (t, J = 7.6 Hz, 1H), 7.07 (d, J = 7.6 |
| | Hz, 2H), 6.25 (s, 1H), 5.42 (dd, J = 11.3, 4.5 Hz, 1H), 5.07 (d, |
| | J = 15.3 Hz, 1H), 4.44 (t, J = 11.1 Hz, 1H), 4.10 (t, J = 11.5 |
| | Hz, 1H), 3.92 (d, J = 15.4 Hz, 1H), 3.35 (d, J = 7.5 Hz, 2H), |
| | 3.19 (s, 3H), 2.09 (dt, J = 13.7, 7.0 Hz, 1H), 2.03 (s, 6H), 1.77 |
| | (d, J = 15.4 Hz, 1H), 1.40 (dd, J = 15.1, 10.0 Hz, 1H), 0.93 (d, |
| | J = 6.5 Hz, 3H), 0.91 (d, J = 6.5 Hz, 3H), 0.45 (s, 3H), 0.33 (dt, |
| | J = 9.7, 5.0 Hz, 1H), 0.23 (dt, J = 9.8, 5.1 Hz, 1H), 0.11 (dt, J = |
| | 9.5, 4.9 Hz, 1H), -0.00 - - 0.04 (m, 1H). |
| 1712 | ¹H NMR (400 MHZ, CDC13) 8 8.74 (t, J = 1.8 Hz, 1H), 8.01 |
| | (dt, J = 7.9, 1.5 Hz, 1H), 7.88 (d, J = 8.2 Hz, 2H), 7.87 - 7.83 |
| | (m, 1H), 7.63 (t, J = 7.8 Hz, 1H), 7.22 (t, J = 7.6 Hz, 1H), 7.07 |
| | (d, J = 7.6 Hz, 2H), 6.25 (s, 1H), 5.38 (d, J = 7.1 Hz, 1H), 5.14 |
| | (d, J = 15.4 Hz, 1H), 4.14 - 4.03 (m, 2H), 4.01 (d, J = 15.4 Hz, |
| | 1H), 3.40 - 3.27 (m, 2H), 3.18 (s, 3H), 2.13 - 2.07 (m, 1H), 2.05 |
| | (s, 6H), 2.01 - 1.94 (m, 1H), 1.82 - 1.73 (m, 2H), 1.68 - 1.61 |
| | (m, 4H), 1.59 - 1.55 (m, 1H), 1.11 - 0.97 (m, 1H), 0.92 (d, J = |
| | 6.4 Hz, 3H), 0.90 (d, J = 6.5 Hz, 3H). |
| 1713 | 1¹H NMR (400 MHZ, CDC13) 8 8.76 (t, J = 1.7 Hz, 1H), 8.02 |
| | (d, J = 7.8 Hz, 1H), 7.91 (s, 1H), 7.84 (d, J = 8.7 Hz, 1H), 7.83 |
| | (s, 1H), 7.65 (t, J = 7.7 Hz, 1H), 7.23 (t, J = 7.6 Hz, 1H), 7.08 |
| | (d, J = 7.6 Hz, 2H), 6.24 (s, 1H), 5.53 - 5.40 (m, 1H), 5.10 (d, |
| | J = 14.9 Hz, 1H), 4.20 (d, J = 12.9 Hz, 2H), 4.08 (t, J = 11.2 |
| | Hz, 1H), 3.40 (d, J = 7.4 Hz, 2H), 3.26 (s, 3H), 2.13 - 2.07 (m, |
| | 1H), 2.05 (s, 6H), 1.70 (t, J = 12.0 Hz, 1H), 1.52 - 1.33 (m, 2H), |
| | 0.94 (d, J = 6.4 Hz, 3H), 0.92 (d, J = 6.4 Hz, 3H), 0.84 (d, J = |
| | 6.2 Hz, 3H), 0.40 (d, J = 6.0 Hz, 3H). |
| 1714 | ¹H NMR (400 MHZ, Chloroform-d) 8 8.84 - 8.79 (m, 1H), 8.37 |
| | (broad s, 1H), 8.14 (s, 1H), 8.00 (d, J = 7.9 Hz, 1H), 7.86 (d, J = |
| | 7.7 Hz, 1H), 7.62 (t, J = 7.8 Hz, 1H), 7.22 (t, J = 7.6 Hz, 1H), |
| | 7.07 (d, J = 7.6 Hz, 2H), 6.27 (s, 1H), 5.45 (dd, J = 11.1, 4.1 |
| | Hz, 1H), 5.06 (d, J = 15.2 Hz, 1H), 4.29 - 4.03 (m, 4H), 2.90 |
| | (s, 3H), 2.53 (s, 3H), 2.19 - 2.09 (m, 4H), 2.03 (s, 6H), 1.76 - |
| | 1.59 (m, 4H), 0.61 (s, 9H). |
| 1322 | ¹H NMR (400 MHZ, Chloroform-d) 8 8.79 (t, J = 1.9 Hz, 1H), |
| | 8.33 (broad s, 1H), 8.10 (s, 1H), 8.01 (d, J = 8.0 Hz, 1H), 7.86 |
| | (d, J = 7.7 Hz, 1H), 7.63 (t, J = 7.8 Hz, 1H), 7.21 (t, J = 7.6 |
| | Hz, 1H), 7.07 (d, J = 7.6 Hz, 2H), 6.28 (s, 1H), 5.49 (dd, J = |
| | 10.3, 3.3 Hz, 1H), 5.02 (d, J = 15.1 Hz, 1H), 4.28 - 4.03 (m, |
| | 3H), 3.22 - 3.06 (m, 2H), 3.02 (s, 3H), 2.55 (s, 3H), 2.10 - 1.92 |
| | (m, 7H), 1.71 (dd, J = 15.0, 8.2 Hz, 1H), 1.55 (d, J = 15.1 Hz, |
| | 1H), 0.87 - 0.78 (m, 6H), 0.61 (s, 9H). |
| 1715 | ¹H NMR (400 MHZ, CDC13) 8 8.80 (s, 1H), 8.12 (d, J = 7.3 Hz, |
| | 1H), 8.00 (d, J = 7.9 Hz, 1H), 7.86 (dt, J = 7.7, 1.4 Hz, 1H), |
| | 7.68 (t, J = 7.8 Hz, 1H), 7.23 (t, J = 7.6 Hz, 1H), 7.06 (d, J = |
| | 7.6 Hz, 2H), 6.51 (s, 1H), 6.23 (s, 1H), 5.60 - 5.26 (m, 3H), 4.59 |
| | (d, J = 17.7 Hz, 1H), 4.18 - 4.02 (m, 1H), 4.02 - 3.88 (m, 1H), |
| | 3.12 (s, 3H), 2.04 (s, 6H), 1.71 - 1.62 (m, 3H), 1.34 (s, 6H), 1.20 - |
| | 1.14 (m, 1H), 0.80 (s, 9H). |
| 1716 | ¹H NMR (400 MHZ, CDC13) 8 8.72 (s, 1H), 8.03 (d, J = 8.0 Hz, |
| | 1H), 7.92 (s, 1H), 7.86 - 7.82 (m, 1H), 7.82 (s, 1H), 7.61 (t, J = |
| | 7.2 Hz, 1H), 7.20 (t, J = 7.6 Hz, 1H), 7.05 (d, J = 7.6 Hz, 2H), |
| | 6.22 (s, 1H), 5.43 (dd, J = 11.2, 4.0 Hz, 1H), 5.11 (d, J = 15.1 |
| | Hz, 1H), 4.16 (t, J = 11.4 Hz, 1H), 4.09 - 3.99 (m, 2H), 3.96 (d, |
| | J = 15.1 Hz, 1H), 3.66 - 3.50 (m, 2H), 2.42 - 2.30 (m, 1H), 2.03 |
| | (s, 6H), 2.01 - 1.98 (m, 1H), 1.96 - 1.91 (m, 2H), 1.68 - 1.57 |
| | (m, 3H), 1.16 (dq, J = 12.3, 5.3 Hz, 1H), 0.91 (d, J = 7.0 Hz, |
| | 3H), 0.83 (d, J = 6.8 Hz, 3H), 0.81 (s, 9H), 0.77 - 0.72 (m, 1H). |
| 1717 | ¹H NMR (400 MHZ, Chloroform-d) 8 8.75 (d, J = 1.8 Hz, 1H), |
| | 7.98 (d, J = 7.9 Hz, 1H), 7.88 (d, J = 2.6 Hz, 2H), 7.84 (dt, J = |
| | 7.7, 1.3 Hz, 1H), 7.63 (t, J = 7.8 Hz, 1H), 7.22 (t, J = 7.6 Hz, |
| | 1H), 7.07 (d, J = 7.6 Hz, 2H), 6.24 (s, 1H), 5.41 (dd, J = 10.7, |
| | 3.7 Hz, 1H), 5.13 (d, J = 15.5 Hz, 1H), 4.16 (dt, J = 8.6, 5.6 |
| | Hz, 1H), 4.13 - 4.00 (m, 2H), 3.42 - 3.32 (m, 2H), 3.20 (s, 3H), |
| | 2.05 (s, 8H), 1.87 - 1.71 (m, 3H), 1.03 (q, J = 9.0, 8.4 Hz, 2H), |
| | 0.92 (dd, J = 6.7, 4.2 Hz, 6H), 0.46 - 0.34 (m, 1H), 0.29 (qt, J = |
| | 6.6, 3.5 Hz, 2H). |
| 1718 | ¹H NMR (400 MHZ, Chloroform-d) 8 8.76 (s, 1H), 7.94 (d, J = |
| | 7.8 Hz, 1H), 7.90 (s, 1H), 7.87 (s, 1H), 7.83 (d, J = 7.8, 1.4 Hz, |
| | 1H), 7.62 (t, J = 7.8 Hz, 1H), 7.22 (t, J = 7.6 Hz, 1H), 7.07 (d, |
| | J = 7.6 Hz, 2H), 6.24 (s, 1H), 5.41 (dd, J = 11.0, 3.9 Hz, 1H), |
| | 5.11 (d, J = 15.6 Hz, 1H), 4.72 (p, J = 6.6 Hz, 1H), 4.22 - 4.13 |
| | (m, 1H), 4.11 - 4.03 (m, 2H), 2.97 (s, 3H), 2.05 (s, 6H), 1.82 - |
| | 1.72 (m, 2H), 1.26 (d, J = 6.6 Hz, 3H), 1.21 (d, J = 6.6 Hz, 3H), |
| | 1.03 (q, J = 7.4 Hz, 2H), 0.45 - 0.34 (m, 1H), 0.32 - 0.22 (m, |
| | 2H), -0.15 (d, J = 4.9 Hz, 2H). |
| 1719 | ¹H NMR (400 MHZ, Chloroform-d) 8 8.75 (s, 1H), 7.99 (s, 1H), |
| | 7.94 (d, J = 7.9 Hz, 1H), 7.88 (s, 1H), 7.83 (d, J = 7.7, 1.4 Hz, |
| | 1H), 7.62 (t, J = 7.8 Hz, 1H), 7.22 (t, J = 7.6 Hz, 1H), 7.06 (d, |
| | J = 7.6 Hz, 2H), 6.21 (s, 1H), 5.37 (dd, J = 10.8, 3.8 Hz, 1H), |
| | 5.17 (d, J = 15.4 Hz, 1H), 4.20 - 4.10 (m, 1H), 4.09 - 3.98 (m, |
| | 2H), 3.50 - 3.35 (m, 2H), 3.21 (s, 3H), 2.03 (s, 6H), 1.81 (d, J = |
| | 9.1 Hz, 2H), 1.08 - 1.00 (m, 2H), 0.98 (s, 9H), 0.45 - 0.36 (m, |
| | 1H), 0.34 - 0.22 (m, 2H), -0.09 - - 0.18 (m, 2H). |
| 1720 | ¹H NMR (400 MHZ, Chloroform-d) 8 8.72 (s, 1H), 7.92 (d, J = |
| | 7.9 Hz, 1H), 7.89 (s, 1H), 7.83 (d, J = 7.7, 1.4 Hz, 1H), 7.78 (s, |
| | 1H), 7.61 (t, J = 7.8 Hz, 1H), 7.23 (t, J = 7.6 Hz, 1H), 7.07 (d, |
| | J = 7.6 Hz, 2H), 6.25 (s, 1H), 5.44 (d, J = 6.8 Hz, 1H), 5.09 (d, |
| | J = 15.3 Hz, 1H), 4.19 - 4.11 (m, 2H), 4.05 (d, J = 15.2 Hz, |
| | 2H), 3.67 - 3.56 (m, 2H), 2.38 - 2.28 (m, 1H), 2.05 (s, 8H), 1.99 |
| | (s, 2H), 1.78 (t, J = 5.7 Hz, 2H), 1.03 (q, J = 7.6, 6.8 Hz, 2H), |
| | 0.92 (d, J = 6.9 Hz, 3H), 0.82 (d, J = 6.8 Hz, 3H), 0.45 - 0.35 |
| | (m, 1H), 0.30 - 0.25 (m, 2H), -0.14 (d, J = 4.8 Hz, 2H). |
| 1726 | ¹H NMR (400 MHZ, Chloroform-d) 8 8.72 (s, 1H), 8.02 (d, J = |
| | 7.8 Hz, 1H), 7.93 (s, 1H), 7.83 (d, J = 7.4 Hz, 1H), 7.65 (t, J = |
| | 7.8 Hz, 2H), 7.22 (t, J = 7.6 Hz, 1H), 7.07 (d, J = 7.5 Hz, 2H), |
| | 6.26 - 6.21 (m, 1H), 5.46 (d, J = 9.5 Hz, 1H), 5.07 (d, J = 13.8 |
| | Hz, 1H), 4.62 - 4.52 (m, 1H), 4.23 - 4.00 (m, 5H), 2.66 - 2.57 |
| | (m, 1H), 2.19 - 2.12 (m, 1H), 2.10 - 2.01 (m, 7H), 1.69 - 1.60 |
| | (m, 2H), 1.58 (d, J = 5.3 Hz, 3H), 1.19 - 1.10 (m, 1H), 0.81 (s, |
| | 9H). Sulfonamide NH not vivsible. |
| 1728 | ¹H NMR (400 MHz, Chloroform-d) 8 8.79 (s, 1H), 7.95 (s, 1H), |
| | 7.91 (t, J = 7.0 Hz, 1H), 7.82 (d, J = 7.7 Hz, 1H), 7.70 (s, 1H), |
| | 7.59 - 7.50 (m, 1H), 7.20 (t, J = 7.6 Hz, 1H), 7.05 (d, J = 7.6 |
| | Hz, 2H), 6.19 (s, 1H), 5.40 (dd, J = 11.2, 4.3 Hz, 1H), 5.06 (d, |
| | J = 15.3 Hz, 1H), 4.34 - 4.24 (m, 2H), 4.24 - 4.15 (m, 1H), 4.11 - |
| | 4.01 (m, 2H), 2.79 - 2.68 (m, 1H), 2.00 (s, 6H), 1.76 - 1.64 |
| | (m, 2H), 1.57 (d, J = 6.2 Hz, 3H), 1.53 (s, 1H), 1.49 (d, J = 6.1 |
| | Hz, 3H), 0.61 (s, 9H). |
| 1729 | ¹H NMR (400 MHZ, Chloroform-d) 8 8.85 (s, 1H), 8.03 (d, J = |
| | 7.6 Hz, 1H), 7.88 (s, 2H), 7.84 (d, 1H), 7.66 - 7.59 (m, 1H), |
| | 6.88 (s, 2H), 6.21 - 6.16 (m, 1H), 5.45 (dd, J = 11.3, 4.2 Hz, |
| | 1H), 5.09 (d, J = 15.5 Hz, 1H), 4.59 - 4.48 (m, 1H), 4.27 - 4.17 |
| | (m, 1H), 4.11 (d, J = 15.5 Hz, 1H), 4.03 (t, J = 11.1, 3.0 Hz, |
| | 1H), 3.15 (s, 3H), 2.30 (s, 6H), 2.24 - 2.15 (m, 2H), 1.98 (s, |
| | 6H), 1.73 - 1.67 (m, 2H), 1.53 (d, J = 15.0 Hz, 1H), 0.62 (s, |
| | 9H). |
| 1730 | ¹H NMR (400 MHZ, Chloroform-d) 8 8.72 (s, 1H), 8.02 (d, J = |
| | 8.0 Hz, 1H), 7.99 (s, 1H), 7.94 (s, 1H), 7.83 (dt, J = 7.6, 1.4 Hz, |
| | 1H), 7.65 (t, J = 7.8 Hz, 1H), 7.21 (t, J = 7.6 Hz, 1H), 7.06 (d, |
| | J = 7.6 Hz, 2H), 6.22 (s, 1H), 5.44 (dd, J = 11.3, 3.9 Hz, 1H), |
| | 5.10 (d, J = 15.3 Hz, 1H), 4.53 (p, J = 6.7 Hz, 1H), 4.19 - 3.96 |
| | (m, 5H), 3.76 - 3.69 (m, 2H), 3.40 (s, 3H), 2.54 - 2.42 (m, 1H), |
| | 2.37 - 2.29 (m, 1H), 2.04 (s, 6H), 1.65 - 1.57 (m, 3H), 1.19 - |
| | 1.12 (m, 1H), 0.81 (s, 9H). Sulfonamide NH not visible. |
| 1731 | ¹H NMR (400 MHZ, Chloroform-d) 8 8.90 (t, J = 1.8 Hz, 1H), |
| | 8.02 (dt, J = 7.6, 1.4 Hz, 1H), 7.87 (s, 2H), 7.74 (s, 1H), 7.70 |
| | (t, J = 7.8 Hz, 1H), 7.41 - 7.27 (m, 5H), 7.19 (t, J = 7.6 Hz, |
| | 1H), 7.03 (d, J = 7.6 Hz, 2H), 6.10 (s, 1H), 5.66 (dd, J = 11.9, |
| | 4.4 Hz, 1H), 5.35 (dd, J = 11.8, 4.4 Hz, 1H), 4.87 - 4.75 (m, |
| | 2H), 4.56 (p, J = 8.6 Hz, 1H), 3.74 (d, J = 16.0 Hz, 1H), 3.18 |
| | (s, 3H), 2.33 (td, J = 7.5, 3.0 Hz, 2H), 2.22 (p, J = 10.0 Hz, |
| | 2H), 1.99 (s, 6H), 1.75 (s, 2H). Sulfonamide NH not visible. |
| 1733 | ¹H NMR (400 MHZ, Chloroform-d) 8 8.84 - 8.80 (m, 1H), 8.07 |
| | (dd, J = 8.0, 1.7 Hz, 1H), 7.89 (s, 2H), 7.87 (dt, J = 7.6, 1.4 Hz, |
| | 1H), 7.67 (t, J = 7.8 Hz, 1H), 7.21 (t, J = 7.6 Hz, 1H), 7.07 (d, |
| | J = 7.6 Hz, 2H), 6.22 (s, 1H), 5.48 (dd, J = 11.9, 4.0 Hz, 1H), |
| | 5.19 (d, J = 15.5 Hz, 1H), 4.53 (p, J = 8.5 Hz, 1H), 4.39 (t, J = |
| | 11.8 Hz, 1H), 4.06 - 3.95 (m, 2H), 3.14 (s, 3H), 2.32 - 2.17 (m, |
| | 4H), 2.06 (s, 6H), 1.91 - 1.65 (m, 8H), 1.26 - 1.16 (m, 2H), 1.07 - |
| | 0.97 (m, 1H), 0.83 - 0.70 (m, 2H). Sulfonamide NH not |
| | visible. |
| 1735 | ¹H NMR (400 MHZ, Chloroform-d) 8 8.71 (s, 1H), 7.93 (d, J = |
| | 7.7 Hz, 1H), 7.90 (s, 1H), 7.83 (d, J = 7.7, 1.5 Hz, 1H), 7.80 (s, |
| | 1H), 7.62 (t, 1H), 7.22 (t, J = 7.6 Hz, 1H), 7.06 (d, J = 7.6 Hz, |
| | 2H), 6.22 (s, 1H), 5.42 (dd, J = 10.3, 3.2 Hz, 1H), 5.12 (d, J = |
| | 15.2 Hz, 1H), 4.15 - 3.98 (m, 4H), 3.66 - 3.55 (m, 2H), 2.38 - |
| | 2.29 (m, 1H), 2.04 (s, 8H), 1.99 - 1.94 (m, 2H), 1.73 - 1.59 (m, |
| | 2H), 1.37 - 1.25 (m, 1H), 1.17 - 1.06 (m, 1H), 0.92 (d, J = 7.0 |
| | Hz, 3H), 0.83 - 0.76 (m, 10H). |
| 1736 | ¹H NMR (400 MHZ, Chloroform-d) 8 8.73 (s, 1H), 7.97 (d, J = |
| | 9.0 Hz, 2H), 7.82 (d, J = 7.6 Hz, 1H), 7.66 (s, 1H), 7.60 (t, J = |
| | 7.9 Hz, 1H), 7.21 (t, J = 7.6 Hz, 1H), 7.06 (d, J = 7.6 Hz, 2H), |
| | 6.22 (s, 1H), 5.44 (dd, J = 10.7, 3.4 Hz, 1H), 5.08 (d, J = 15.3 |
| | Hz, 1H), 4.54 - 4.44 (m, 1H), 4.14 - 4.08 (m, 2H), 4.02 - 3.97 |
| | (m, 2H), 2.54 (ddt, J = 16.8, 8.7, 4.7 Hz, 1H), 2.04 (s, 6H), 1.67 - |
| | 1.60 (m, 3H), 1.56 (d, J = 6.2 Hz, 3H), 1.34 - 1.25 (m, 2H), |
| | 1.15 - 1.07 (m, 1H), 0.93 - 0.85 (m, 1H), 0.77 (dd, J = 10.8, 6.6 |
| | Hz, 6H). |
| 1737 | ¹H NMR (400 MHZ, Chloroform-d) & 8.69 (s, 1H), 8.00 (s, 1H), |
| | 7.90 (s, 2H), 7.69 (s, 1H), 7.14 (t, J = 7.7 Hz, 1H), 7.00 (s, 2H), |
| | 6.12 (s, 1H), 5.45 (d, J = 7.3 Hz, 1H), 5.08 (d, J = 15.3 Hz, |
| | 1H), 4.57 - 4.48 (m, 1H), 4.17 - 4.09 (m, 1H), 4.09 - 3.96 (m, |
| | 4H), 3.72 (d, J = 4.9 Hz, 2H), 3.35 (s, 3H), 2.44 (s, 1H), 2.35 - |
| | 2.25 (m, 1H), 1.96 (s, 6H), 1.66 (s, 3H), 1.32 - 1.25 (m, 1H), |
| | 1.16 - 1.06 (m, 1H), 0.94 - 0.83 (m, 1H), 0.78 (dd, J = 11.7, 6.6 |
| | Hz, 6H). |
| 1738 | ¹H NMR (400 MHZ, Chloroform-d) 8 8.74 (s, 1H), 8.00 - 7.87 |
| | (m, 2H), 7.67 (s, 1H), 7.59 (s, 1H), 7.10 (t, J = 7.8 Hz, 1H), |
| | 6.96 (d, J = 7.4 Hz, 2H), 6.10 (s, 1H), 5.49 (d, J = 8.6 Hz, 1H), |
| | 4.93 (d, J = 15.1 Hz, 1H), 4.58 (q, J = 6.3 Hz, 2H), 4.20 - 4.11 |
| | (m, 1H), 4.07 (d, J = 15.1 Hz, 2H), 2.15 (t, J = 6.6 Hz, 2H), |
| | 1.92 (s, 6H), 1.71 (dd, J = 15.1, 8.4 Hz, 2H), 1.55 (d, J = 14.9 |
| | Hz, 1H), 1.46 (d, J = 6.1 Hz, 6H), 0.63 (s, 9H). |
| 1739 | ¹H NMR (400 MHZ, CDC13) 8 8.73 (t, J = 1.8 Hz, 1H), 8.05 (d, |
| | J = 7.9 Hz, 1H), 8.02 (s, 1H), 7.86 (d, J = 8.6 Hz, 1H), 7.84 (dt, |
| | J = 7.7, 1.4 Hz, 1H), 7.65 (t, J = 7.8 Hz, 1H), 7.20 (t, J = 7.6 |
| | Hz, 1H), 7.02 (d, J = 7.6 Hz, 2H), 6.16 (s, 1H), 5.42 - 5.29 (m, |
| | 1H), 5.21 (d, J = 15.4 Hz, 1H), 4.06 - 3.99 (m, 2H), 3.95 (d, J = |
| | 15.5 Hz, 1H), 3.46 (d, J = 14.7 Hz, 1H), 3.38 (d, J = 14.7 Hz, |
| | 1H), 3.20 (s, 3H), 1.99 (s, 6H), 1.69 - 1.53 (m, 3H), 1.21 - 1.11 |
| | (m, 1H), 0.98 (s, 9H), 0.82 (s, 9H). |
| 1740 | ¹H NMR (400 MHZ, MeOD) 8 8.63 (s, 1H), 7.96 (d, J = 7.8 Hz, |
| | 2H), 7.87 (s, 1H), 7.79 - 7.68 (m, 1H), 7.65 (t, J = 7.6 Hz, 1H), |
| | 7.28 (t, J = 7.6 Hz, 1H), 7.15 (d, J = 7.7 Hz, 2H), 6.50 - 5.77 |
| | (m, 2H), 5.60 - 4.97 (m, 2H), 4.77 - 4.61 (m, 1H), 4.61 - 4.14 |
| | (m, 2H), 4.13 - 3.86 (m, 1H), 3.79 - 3.62 (m, 1H), 3.61 - 3.43 |
| | (m, 1H), 3.19 (s, 3H), 3.14 - 2.83 (m, 1H), 2.82 - 2.40 (m, 1H), |
| | 2.36 - 2.23 (m, 4H), 2.12 (s, 6H), 1.83 - 1.70 (m, 2H), 1.02 |
| | (broad, 9H). |
| 1741 | ¹H NMR (400 MHZ, MeOD) 8 8.59 (broad, 1H), 8.08 (s, 1H), |
| | 7.98 (d, J = 7.0 Hz, 2H), 7.73 (d, J = 7.6 Hz, 1H), 7.67 (t, J = |
| | 7.6 Hz, 1H), 7.29 (t, J = 7.6 Hz, 1H), 7.15 (d, J = 7.6 Hz, 2H), |
| | 6.32 (d, J = 2.4 Hz, 1H), 6.26 (s, 1H), 5.16 (d, J = 16.8 Hz, |
| | 1H), 4.91 - 4.87 (m, 1H), 4.78 - 4.65 (m, 1H), 4.61 (d, J = 17.5 |
| | Hz, 1H), 4.35 (d, J = 14.5 Hz, 1H), 4.24 (d, J = 14.6 Hz, 1H), |
| | 3.95 (d, J = 13.9 Hz, 1H), 3.90 - 3.79 (m, 1H), 3.62 - 3.39 (m, |
| | 2H), 3.24 (s, 3H), 2.39 - 2.25 (m, 4H), 2.12 (s, 6H), 1.89 - 1.68 |
| | (m, 4H), 0.96 (s, 9H) |
| 1742 | ¹H NMR (400 MHZ, MeOD) & 8.60 (s, 1H), 8.09 (s, 1H), 8.03 - |
| | 7.94 (m, 2H), 7.72 (d, J = 7.6 Hz, 1H), 7.67 (t, J = 7.6 Hz, |
| | 1H), 7.29 (t, J = 7.6 Hz, 1H), 7.16 (d, J = 7.6 Hz, 2H), 6.45 (s, |
| | 1H), 6.33 (d, J = 2.5 Hz, 1H), 5.21 (d, J = 16.9 Hz, 1H), 4.91 |
| | (d, J = 7.0 Hz, 1H), 4.73 - 4.59 (m, 2H), 4.49 - 4.38 (m, 1H), |
| | 4.33 (d, J = 14.3 Hz, 1H), 4.16 (d, J = 14.3 Hz, 1H), 3.81 (d, J |
| | = 15.0 Hz, 1H), 3.78 - 3.66 (m, 1H), 3.24 (s, 3H), 2.80 (dd, J = |
| | 12.0, 7.5 Hz, 1H), 2.76 - 2.66 (m, 1H), 2.42 - 2.25 (m, 6H), 2.13 |
| | (s, 6H), 1.84 - 1.72 (m, 2H), 0.54 (s, 4H). |
| 1323 | ¹H NMR (400 MHZ, MeOD) 8 8.59 (s, 1H), 8.03 (s, 1H), 8.00 - |
| | 7.96 (m, 1H), 7.94 (s, 1H), 7.72 (d, J = 7.6 Hz, 1H), 7.66 (t, J = |
| | 7.7 Hz, 1H), 7.29 (t, J = 7.6 Hz, 1H), 7.16 (d, J = 7.7 Hz, |
| | 2H), 6.41 (s, 1H), 6.33 (d, J = 2.5 Hz, 1H), 5.14 (d, J = 16.7 |
| | Hz, 1H), 4.65 (p, J = 8.4 Hz, 1H), 4.49 (d, J = 16.7 Hz, 1H), |
| | 4.30 (d, J = 14.2 Hz, 1H), 4.23 - 4.05 (m, 2H), 3.78 (d, J = 14.2 |
| | Hz, 1H), 3.72 (dd, J = 14.3, 7.1 Hz, 1H), 3.20 (s, 3H), 2.40 - |
| | 2.24 (m, 8H), 2.13 (s, 6H), 1.83 - 1.72 (m, 2H), 1.72 - 1.62 (m, |
| | 5H), 1.61 - 1.52 (m, 4H) |
| 1746 | ¹H NMR (400 MHZ, Chloroform-d) 8 8.82 (s, 1H), 7.99 (d, J = |
| | 8.0 Hz, 1H), 7.90 (s, 1H), 7.87 - 7.83 (m, 2H), 7.64 (t, J = 7.8 |
| | Hz, 1H), 7.21 (t, J = 7.6 Hz, 1H), 7.05 (d, J = 7.6 Hz, 2H), 6.20 |
| | (s, 1H), 5.35 (dd, J = 11.1, 4.0 Hz, 1H), 5.25 (d, J = 15.9 Hz, |
| | 1H), 4.41 (q, J = 7.8 Hz, 1H), 4.13 - 4.07 (m, 1H), 4.03 (d, J = |
| | 15.9 Hz, 1H), 3.95 (d, J = 11.3 Hz, 1H), 3.92 - 3.84 (m, 1H), |
| | 3.39 - 3.31 (m, 1H), 2.69 (s, 1H), 2.62 (s, 1H), 2.35 - 2.26 (m, |
| | 1H), 2.02 (s, 6H), 2.01 - 1.89 (m, 4H), 1.68 - 1.62 (m, 2H), 1.55 |
| | (d, J = 8.1 Hz, 2H), 1.35 - 1.26 (m, 1H), 1.17 - 1.06 (m, 1H), |
| | 0.94 - 0.84 (m, 1H), 0.78 (dd, J = 9.4, 6.6 Hz, 6H). |
| 1747 | ¹H NMR (400 MHZ, Chloroform-d) 8 8.74 (s, 1H), 7.94 (d, J = |
| | 7.9 Hz, 1H), 7.90 (s, 1H), 7.86 (s, 1H), 7.83 (d, J = 7.6, 1.4 Hz, |
| | 1H), 7.62 (t, J = 7.8 Hz, 1H), 7.22 (t, J = 7.6 Hz, 1H), 7.05 (d, |
| | J = 7.6 Hz, 2H), 6.19 (s, 1H), 5.37 (d, 1H), 5.16 (d, J = 15.4 |
| | Hz, 1H), 4.35 (q, J = 7.8 Hz, 1H), 4.09 - 3.91 (m, 4H), 3.36 - |
| | 3.29 (m, 1H), 2.70 (s, 1H), 2.62 (s, 2H), 2.41 - 2.32 (m, 1H), |
| | 2.02 (s, 6H), 2.00 - 1.92 (m, 3H), 1.64 - 1.55 (m, 4H), 1.35 - |
| | 1.27 (m, 1H), 1.16 - 1.06 (m, 1H), 0.94 - 0.85 (m, 1H), 0.78 (dd, |
| | J = 9.0, 6.6 Hz, 6H). |
| 1748 | ¹H NMR (400 MHZ, Chloroform-d) 8 14.13 (s, 1H), 8.67 (s, |
| | 1H), 8.20 (d, J = 7.9 Hz, 1H), 7.88 (d, J = 7.7, 1.4 Hz, 1H), |
| | 7.79 - 7.69 (m, 2H), 7.22 (t, J = 7.6 Hz, 1H), 7.04 (d, J = 7.7 |
| | Hz, 2H), 6.86 (d, J = 7.4 Hz, 1H), 6.69 (d, J = 9.1 Hz, 1H), 6.20 |
| | (s, 1H), 5.76 (d, J = 17.4 Hz, 1H), 5.41 (d, J = 17.3 Hz, 1H), |
| | 5.12 (dd, J = 10.4, 4.0 Hz, 1H), 4.48 (s, 1H), 4.23 - 4.14 (m, |
| | 1H), 3.76 (s, 1H), 2.62 (s, 2H), 2.33 - 2.14 (m, 6H), 2.01 (s, |
| | 6H), 1.65 - 1.55 (m, 1H), 1.35 (d, J = 6.3 Hz, 3H), 1.31 - 1.22 |
| | (m, 1H), 1.14 - 1.03 (m, 1H), 0.75 (d, J = 6.5 Hz, 6H). |
| 1750 | ¹H NMR (400 MHZ, Chloroform-d) 8 8.86 (s, 1H), 8.00 - 7.93 |
| | (m, 2H), 7.87 - 7.80 (m, 2H), 7.61 (t, J = 7.8 Hz, 1H), 7.22 (t, |
| | J = 7.6 Hz, 1H), 7.07 (d, J = 7.6 Hz, 2H), 6.23 (s, 1H), 5.46 |
| | (dd, J = 11.3, 4.3 Hz, 1H), 5.08 (d, J = 15.6 Hz, 1H), 4.23 - |
| | 4.14 (m, 1H), 4.11 (d, J = 15.6 Hz, 1H), 4.02 - 3.92 (m, 2H), |
| | 3.69 (d, J = 8.5 Hz, 1H), 3.49 (d, J = 9.7 Hz, 1H), 2.26 - 2.16 |
| | (m, 1H), 2.01 (s, 6H), 1.68 (dd, J = 15.1, 8.8 Hz, 1H), 1.53 (d, |
| | J = 15.1, 1.6 Hz, 1H), 1.29 (s, 3H), 1.18 (s, 3H), 1.03 (d, J = |
| | 6.8 Hz, 3H), 0.95 (d, J = 6.5 Hz, 3H), 0.61 (s, 9H). |
| 1751 | ¹H NMR (400 MHz, Chloroform-d) 8 8.77 (s, 1H), 8.03 - 7.97 |
| | (m, 2H), 7.85 (d, J = 7.7, 1.4 Hz, 1H), 7.79 (s, 1H), 7.63 (t, J = |
| | 7.8 Hz, 1H), 7.22 (t, J = 7.6 Hz, 1H), 7.07 (d, J = 7.6 Hz, 2H), |
| | 6.27 (s, 1H), 5.41 (dd, J = 10.5, 3.6 Hz, 1H), 5.02 (d, J = 15.2 |
| | Hz, 1H), 4.24 - 4.08 (m, 3H), 3.93 (d, J = 8.4 Hz, 1H), 3.68 (d, |
| | J = 8.4 Hz, 1H), 3.61 (d, J = 9.5 Hz, 1H), 2.27 - 2.17 (m, 2H), |
| | 2.02 (s, 6H), 1.70 (dd, J = 15.2, 8.4 Hz, 1H), 1.54 (d, J = 15.0 |
| | Hz, 1H), 1.27 (d, J = 10.8 Hz, 6H), 1.04 (d, J = 6.8 Hz, 3H), |
| | 0.98 (d, J = 6.5 Hz, 3H), 0.60 (s, 9H). |
| 1752 | ¹H NMR (400 MHz, Chloroform-d) 8 8.91 (s, 1H), 8.02 (d, J = |
| | 7.8, 1.7 Hz, 1H), 7.91 (s, 1H), 7.85 (d, J = 7.6, 1.4 Hz, 1H), |
| | 7.69 (s, 1H), 7.63 (t, J = 7.8 Hz, 1H), 7.22 (t, J = 7.6 Hz, 1H), |
| | 7.07 (d, J = 7.6 Hz, 2H), 6.25 (s, 1H), 5.50 (dd, J = 11.3, 4.3 |
| | Hz, 1H), 5.06 (d, J = 15.7 Hz, 1H), 4.34 (d, J = 9.0 Hz, 1H), |
| | 4.25 - 4.16 (m, 1H), 4.15 - 4.06 (m, 2H), 3.98 (t, J = 11.5 Hz, |
| | 1H), 3.81 (d, J = 6.3 Hz, 1H), 2.49 - 2.38 (m, 1H), 2.19 - 2.13 |
| | (m, 1H), 2.10 - 2.04 (m, 3H), 2.03 (s, 6H), 1.99 - 1.94 (m, 1H), |
| | 1.81 - 1.74 (m, 1H), 1.70 - 1.62 (m, 1H), 1.55 (d, J = 15.1 Hz, |
| | 1H), 1.09 - 1.01 (m, 6H), 0.62 (s, 9H). |
| 1753 | ¹H NMR (400 MHZ, Chloroform-d) 8 8.79 (s, 1H), 8.01 (d, J = |
| | 7.9 Hz, 1H), 7.93 (s, 1H), 7.85 (d, J = 7.6 Hz, 1H), 7.67 (s, 1H), |
| | 7.63 (t, J = 7.8 Hz, 1H), 7.23 (t, J = 7.6 Hz, 1H), 7.08 (d, J = |
| | 7.6 Hz, 2H), 6.27 (s, 1H), 5.45 (dd, J = 10.8, 3.9 Hz, 1H), 5.01 |
| | (d, J = 15.3 Hz, 1H), 4.25 (d, J = 9.0 Hz, 1H), 4.22 - 4.17 (m, |
| | 1H), 4.14 (s, 1H), 4.13 - 4.06 (m, 2H), 3.99 (d, J = 5.8 Hz, 1H), |
| | 2.50 - 2.41 (m, 1H), 2.27 - 2.18 (m, 2H), 2.18 - 2.07 (m, 2H), |
| | 2.03 (s, 6H), 1.99 - 1.90 (m, 1H), 1.88 - 1.78 (m, 1H), 1.68 (dd, |
| | J = 15.2, 8.5 Hz, 1H), 1.55 (d, J = 15.0 Hz, 1H), 1.07 (dd, J = |
| | 8.5, 6.9 Hz, 6H), 0.61 (s, 9H). |
| 1755 | ¹H NMR (400 MHZ, DMSO-d6) 8 13.06 (br. s., 1H), 8.79 (br. |
| | s., 1H), 8.52 (s, 1H), 7.95 (br. s., 1H), 7.87 (d, J = 3.7 Hz, 1H), |
| | 7.68 (br. s., 2H), 7.31 - 7.21 (m, 1H), 7.19 - 7.06 (m, 2H), 6.64 |
| | (d, J = 3.4 Hz, 1H), 6.44 (br. s., 1H), 5.61 - 5.48 (m, 1H), 5.05 |
| | (d, J = 16.1 Hz, 1H), 4.74 (d, J = 16.1 Hz, 1H), 4.31 (t, J = 10.6 |
| | Hz, 1H), 4.16 (d, J = 7.3 Hz, 2H), 4.12 - 4.03 (m, 1H), 2.25 - |
| | 1.87 (m, 7H), 1.81 (dd, J = 14.9, 9.0 Hz, 1H), 1.46 (d, J = 14.7 |
| | Hz, 1H), 0.82 (d, J = 6.8 Hz, 3H), 0.74 (d, J = 6.6 Hz, 3H), 0.60 |
| | (s, 9H). |
| 1756 | ¹H NMR (400 MHz, DMSO-d6) 8 12.89 (br. s, 1H), 8.83 (s, |
| | 1H), 8.44 (s, 1H), 7.99 - 7.92 (m, 1H), 7.73 - 7.63 (m, 2H), 7.31 - |
| | 7.23 (m, 1H), 7.13 (d, J = 7.3 Hz, 2H), 6.48 - 6.38 (m, 2H), |
| | 5.59 (dd, J = 10.4, 3.5 Hz, 1H), 5.05 (d, J = 16.1 Hz, 1H), 4.70 |
| | (d, J = 16.1 Hz, 1H), 4.30 (t, J = 11.1 Hz, 1H), 4.09 - 3.98 (m, |
| | 1H), 3.86 (s, 3H), 3.30 - 3.22 (m, 1H, overlapped with water), |
| | 2.05 (br. s, 6H), 1.83 (dd, J = 15.0, 8.9 Hz, 1H), 1.48 (d, J = |
| | 14.7 Hz, 1H), 1.36 - 1.28 (m, 6H), 0.61 (s, 9H). |
| 1757 | ¹H NMR (400 MHZ, Chloroform-d) 8 8.86 (t, J = 1.8 Hz, 1H), |
| | 8.09 - 8.04 (m, 1H), 7.99 (s, 1H), 7.90 (s, 1H), 7.88 (dt, J = 7.8, |
| | 1.4 Hz, 1H), 7.65 (t, J = 7.8 Hz, 1H), 7.21 (t, J = 7.6 Hz, 1H), |
| | 7.07 (d, J = 7.6 Hz, 2H), 6.27 (s, 1H), 5.46 (dd, J = 11.2, 4.3 |
| | Hz, 1H), 5.07 (d, J = 15.4 Hz, 1H), 4.29 - 4.19 (m, 1H), 4.14 - |
| | 4.04 (m, 2H), 3.58 (d, J = 14.6 Hz, 1H), 3.45 (d, J = 14.6 Hz, |
| | 1H), 3.18 (s, 3H), 2.02 (s, 6H), 1.72 (dd, J = 15.1, 8.8 Hz, 1H), |
| | 1.53 (d, J = 15.1 Hz, 1H), 1.01 (s, 3H), 0.61 (s, 9H), 0.48 - 0.44 |
| | (m, 2H), 0.37 - 0.33 (m, 2H). Sulfonamide NH not visible. |
| 1758 | ¹H NMR (400 MHZ, Chloroform-d) 8 8.84 (t, J = 1.8 Hz, 1H), |
| | 8.10 - 8.04 (m, 2H), 7.92 (s, 1H), 7.88 (dt, J = 7.7, 1.3 Hz, 1H), |
| | 7.65 (t, J = 7.8 Hz, 1H), 7.21 (t, J = 7.6 Hz, 1H), 7.07 (d, J = |
| | 7.6 Hz, 2H), 6.24 (s, 1H), 5.39 (dd, J = 11.2, 4.3 Hz, 1H), 5.14 |
| | (d, J = 15.4 Hz, 1H), 4.28 - 4.19 (m, 1H), 4.10 (d, J = 15.5 Hz, |
| | 1H), 4.02 (t, J = 11.4 Hz, 1H), 3.92 - 3.77 (m, 2H), 3.24 (s, 3H), |
| | 3.22 (s, 3H), 2.17 (q, J = 10.1 Hz, 2H), 2.01 (s, 6H), 1.90 - 1.83 |
| | (m, 2H), 1.76 - 1.69 (m, 2H), 1.67 - 1.60 (m, 1H), 1.53 (d, J = |
| | 15.2, 1.7 Hz, 1H), 0.61 (s, 9H). Sulfonamide NH not visible. |
| 1765 | ¹H NMR (400 MHZ, MeOD) 8 8.56 (d, J = 2.0 Hz, 1H), 8.05 |
| | (s, 1H), 8.01 - 7.89 (m, 2H), 7.74 - 7.62 (m, 2H), 7.29 (t, J = |
| | 7.6 Hz, 1H), 7.16 (d, J = 7.6 Hz, 2H), 6.45 - 6.33 (m, 1H), 6.20 - |
| | 6.05 (m, 1H), 5.21 - 4.99 (m, 2H), 4.69 (p, J = 8.3 Hz, 1H), |
| | 4.29 - 3.98 (m, 2H), 3.86 - 3.53 (m, 3H), 3.45 - 3.34 (m, 1H), |
| | 3.22 (s, 3H), 2.83 - 2.52 (m, 1H), 2.41 - 2.23 (m, 5H), 2.13 (s, |
| | 6H), 1.86 - 1.73 (m, 2H), 1.73 - 1.56 (m, 1H), 1.41 - 1.08 (m, |
| | 6H), 0.97 (dd, J = 6.2, 2.3 Hz, 3H). |
| 1766 | ¹H NMR (400 MHZ, MeOD) 8 8.56 (t, J = 1.8 Hz, 1H), 8.09 (s, |
| | 1H), 8.00 (s, 1H), 7.97 (dt, J = 7.6, 1.6 Hz, 1H), 7.74 - 7.69 (m, |
| | 1H), 7.66 (t, J = 7.6 Hz, 1H), 7.29 (t, J = 7.6 Hz, 1H), 7.16 (d, |
| | J = 7.7 Hz, 2H), 6.44 (s, 1H), 6.14 (s, 1H), 5.11 (q, J = 16.4 |
| | Hz, 2H), 4.70 (p, J = 8.5 Hz, 1H), 4.23 (s, 1H), 4.08 (d, J = |
| | 13.9 Hz, 1H), 3.76 (d, J = 14.0 Hz, 1H), 3.64 (d, J = 10.0 Hz, |
| | 2H), 3.50 - 3.33 (m, 2H), 3.25 (s, 3H), 2.73 - 2.53 (m, 1H), 2.43 - |
| | 2.24 (m, 5H), 2.14 (s, 6H), 1.90 - 1.72 (m, 3H), 1.65 (td, J = |
| | 12.4, 4.6 Hz, 1H), 0.98 (s, 9H). |
| 1767 | ¹H NMR (400 MHZ, MeOD) 8 8.56 (t, J = 1.9 Hz, 1H), 8.09 (s, |
| | 1H), 8.00 (s, 1H), 7.97 (dt, J = 7.6, 1.6 Hz, 1H), 7.76 - 7.69 (m, |
| | 1H), 7.66 (t, J = 7.6 Hz, 1H), 7.30 (t, J = 7.6 Hz, 1H), 7.17 (d, |
| | J = 7.6 Hz, 2H), 6.46 (s, 1H), 6.14 (s, 1H), 5.22 - 4.97 (m, 2H), |
| | 4.70 (p, J = 8.4 Hz, 1H), 4.31 (t, J = 8.1 Hz, 1H), 4.26 - 4.14 |
| | (m, 1H), 3.97 (d, J = 13.9 Hz, 1H), 3.67 (d, J = 14.4 Hz, 1H), |
| | 3.64 - 3.57 (m, 1H), 3.52 (t, J = 14.4 Hz, 1H), 3.23 (s, 3H), 2.91 |
| | (dd, J = 11.7, 8.2 Hz, 1H), 2.67 - 2.52 (m, 2H), 2.42 - 2.28 (m, |
| | 6H), 2.24 (t, J = 10.0 Hz, 1H), 2.15 (s, 6H), 1.81 (p, J = 8.3 Hz, |
| | 2H), 0.65 - 0.56 (m, 2H), 0.56 - 0.46 (m, 2H). |
| 1768 | ¹H NMR (400 MHZ, MeOD) 8 8.56 (t, J = 1.9 Hz, 1H), 8.07 (s, |
| | 1H), 7.98 (d, J = 2.6 Hz, 1H), 7.98 - 7.92 (m, 1H), 7.74 - 7.68 |
| | (m, 1H), 7.66 (t, J = 7.6 Hz, 1H), 7.29 (t, J = 7.6 Hz, 1H), 7.16 |
| | (d, J = 7.7 Hz, 2H), 6.51 (s, 1H), 6.12 (s, 1H), 5.17 - 4.96 (m, |
| | 2H), 4.69 (p, J = 8.5 Hz, 1H), 4.27 - 4.16 (m, 1H), 4.04 (p, J = |
| | 8.4 Hz, 1H), 3.92 (d, J = 14.0 Hz, 1H), 3.62 (d, J = 14.3 Hz, |
| | 1H), 3.59 - 3.51 (m, 1H), 3.50 - 3.40 (m, 1H), 3.22 (s, 3H), 2.64 - |
| | 2.51 (m, 2H), 2.41 - 2.29 (m, 5H), 2.28 - 2.24 (m, 1H), 2.24 - |
| | 2.18 (m, 2H), 2.14 (s, 6H), 1.81 (p, J = 8.3 Hz, 2H), 1.72 - 1.61 |
| | (m, 6H), 1.61 - 1.52 (m, 2H). |
| 1769 | ¹H NMR (400 MHZ, Chloroform-d) 8 8.80 (t, J = 1.8 Hz, 1H), |
| | 8.14 (s, 1H), 8.09 (dd, J = 7.9, 1.7 Hz, 1H), 7.89 - 7.84 (m, 1H), |
| | 7.80 (s, 1H), 7.65 (t, J = 7.8 Hz, 1H), 7.21 (t, J = 7.6 Hz, 1H), |
| | 7.08 (d, J = 7.6 Hz, 2H), 6.28 (s, 1H), 5.48 (dd, J = 11.1, 4.2 |
| | Hz, 1H), 5.03 (d, J = 15.4 Hz, 1H), 4.65 (dq, J = 13.2, 6.6 Hz, |
| | 1H), 4.50 (q, J = 10.7 Hz, 1H), 4.45 - 4.32 (m, 1H), 4.27 - 4.18 |
| | (m, 1H), 4.16 - 4.06 (m, 2H), 2.03 (s, 6H), 1.69 (dd, J = 15.1, |
| | 8.6 Hz, 1H), 1.59 (d, J = 6.3 Hz, 3H), 1.57 - 1.54 (m, 1H), 0.62 |
| | (s, 9H). Sulfonamide NH not visible. |
| 1770 | ¹H NMR (400 MHZ, Chloroform-d) 8 8.46 - 8.40 (m, 1H), 7.96 |
| | (dt, J = 8.0, 1.5 Hz, 1H), 7.91 (s, 1H), 7.84 (s, 1H), 7.81 (dt, J = |
| | 7.6, 1.4 Hz, 1H), 7.61 (t, J = 7.8 Hz, 1H), 7.21 (t, J = 7.6 Hz, |
| | 1H), 7.08 (d, J = 7.6 Hz, 2H), 6.21 (s, 1H), 6.02 (t, J = 11.5 Hz, |
| | 1H), 4.83 (d, J = 15.6 Hz, 1H), 4.67 (p, J = 8.7 Hz, 1H), 4.35 |
| | (d, J = 15.6 Hz, 1H), 4.28 - 4.08 (m, 2H), 3.12 (s, 3H), 2.66 (d, |
| | J = 14.7 Hz, 1H), 2.37 - 2.16 (m, 4H), 2.14 - 2.06 (m, 1H), 2.01 |
| | (s, 6H), 1.84 - 1.74 (m, 2H), 1.64 - 1.54 (m, 1H), 1.25 - 1.21 |
| | (m, 1H), 1.19 - 1.07 (m, 1H), 0.61 (d, J = 6.5 Hz, 3H), 0.01 |
| | (overlapped with TMS, d, J = 3.9 Hz, 3H). |
| 1772 | ¹H NMR (400 MHz, Chloroform-d) 8 8.87 (d, J = 1.8 Hz, 1H), |
| | 8.08 (dt, J = 8.0, 1.4 Hz, 1H), 7.93 (s, 1H), 7.89 (dt, J = 7.7, |
| | 1.4 Hz, 1H), 7.83 (s, 1H), 7.66 (t, J = 7.8 Hz, 1H), 7.21 (t, J = |
| | 7.6 Hz, 1H), 7.07 (d, J = 7.6 Hz, 2H), 6.27 (s, 1H), 5.46 (dd, J = |
| | 11.2, 4.3 Hz, 1H), 5.10 (d, J = 15.4 Hz, 1H), 4.31 (q, J = 8.4 |
| | Hz, 1H), 4.26 - 4.18 (m, 1H), 4.15 - 4.04 (m, 2H), 3.21 (s, 3H), |
| | 2.83 (d, J = 7.9 Hz, 1H), 2.61 (p, J = 10.2 Hz, 1H), 2.27 (ddd, |
| | J = 11.0, 9.6, 6.8 Hz, 1H), 2.02 (s, 6H), 1.72 (dd, J = 15.1, 8.7 |
| | Hz, 2H), 1.43 (t, J = 10.5 Hz, 2H), 0.93 (d, J = 7.2 Hz, 3H), |
| | 0.61 (s, 9H). |
| 1773 | ¹H NMR (400 MHZ, Chloroform-d) 8 8.89 (d, J = 1.8 Hz, 1H), |
| | 8.08 (dt, J = 7.9, 1.4 Hz, 1H), 7.92 (s, 1H), 7.88 (dt, J = 7.7, |
| | 1.4 Hz, 1H), 7.81 (s, 1H), 7.65 (t, J = 7.8 Hz, 1H), 7.21 (t, J = |
| | 7.6 Hz, 1H), 7.07 (d, J = 7.6 Hz, 2H), 6.27 (s, 1H), 5.47 (dd, J = |
| | 11.2, 4.3 Hz, 1H), 5.10 (d, J = 15.5 Hz, 1H), 4.35 - 4.18 (m, |
| | 2H), 4.15 - 4.02 (m, 2H), 3.20 (s, 3H), 2.79 (d, J = 8.7 Hz, 1H), |
| | 2.61 (q, J = 10.2 Hz, 1H), 2.32 - 2.21 (m, 1H), 2.10 - 1.90 (m, |
| | 7H), 1.72 (dd, J = 15.1, 8.7 Hz, 2H), 1.40 (t, J = 10.4 Hz, 1H), |
| | 0.92 (d, J = 7.1 Hz, 3H), 0.62 (s, 9H). |
| 1774 | ¹H NMR (400 MHZ, CDC13) 8 8.80 (t, J = 1.9 Hz, 2H), 7.98 |
| | (dt, J = 8.2, 1.3 Hz, 1H), 7.89 (d, J = 10.4 Hz, 2H), 7.82 (dt, J = |
| | 7.7, 1.4 Hz, 1H), 7.61 (t, J = 7.8 Hz, 1H), 7.21 (t, J = 7.6 Hz, |
| | 1H), 7.07 (d, J = 7.6 Hz, 2H), 6.26 (s, 1H), 5.41 (dd, J = 11.0, |
| | 4.2 Hz, 1H), 5.10 (d, J = 15.4 Hz, 1H), 4.28 - 4.15 (m, 1H), |
| | 4.15 - 4.04 (m, 2H), 3.34 (d, J = 7.4 Hz, 2H), 3.18 (s, 3H), 2.13 - |
| | 2.04 (m, 1H), 2.01 (d, J = 3.9 Hz, 6H), 1.90 (dd, J = 15.1, 9.7 |
| | Hz, 1H), 1.85 - 1.71 (m, 2H), 1.68 - 1.62 (m, 2H), 1.57 - 1.46 |
| | (m, 1H), 1.38 (t, J = 7.7 Hz, 2H), 0.92 (d, J = 6.4 Hz, 3H), 0.91 |
| | (d, J = 6.4 Hz, 3H), 0.65 (s, 3H). |
| 1775 | ¹H NMR (400 MHZ, CDC13) 8 8.76 (t, J = 1.9 Hz, 1H), 7.96 - |
| | 7.90 (m, 1H), 7.89 (s, 1H), 7.84 - 7.77 (m, 2H), 7.58 (t, J = 7.8 |
| | Hz, 1H), 7.22 (t, J = 7.6 Hz, 1H), 7.07 (d, J = 7.6 Hz, 2H), 6.25 |
| | (s, 1H), 5.54 - 5.36 (m, 1H), 5.05 (d, J = 15.1 Hz, 1H), 4.25 - |
| | 4.12 (m, 2H), 4.12 - 4.01 (m, 2H), 3.66 - 3.52 (m, 2H), 2.41 - |
| | 2.29 (m, 1H), 2.02 (s, 6H), 2.00 - 1.91 (m, 4H), 1.91 - 1.85 (m, |
| | 1H), 1.85 - 1.79 (m, 1H), 1.79 - 1.71 (m, 1H), 1.71 - 1.60 (m, |
| | 2H), 1.58 - 1.44 (m, 1H), 1.38 (t, J = 7.6 Hz, 2H), 0.92 (d, J = |
| | 7.0 Hz, 3H), 0.81 (d, J = 6.8 Hz, 3H), 0.65 (s, 3H). |
| 1776 | ¹H NMR (400 MHZ, CDC13) 8 14.36 (s, 1H), 8.74 (t, J = 1.8 |
| | Hz, 1H), 8.11 (dt, J = 8.0, 1.4 Hz, 1H), 7.85 (dt, J = 7.7, 1.4 |
| | Hz, 1H), 7.79 - 7.71 (m, 1H), 7.68 (t, J = 7.8 Hz, 1H), 7.22 (t, |
| | J = 7.6 Hz, 1H), 7.05 (d, J = 7.6 Hz, 2H), 6.77 (d, J = 7.4 Hz, |
| | 1H), 6.71 (d, J = 9.1 Hz, 1H), 6.23 (s, 1H), 5.78 (d, J = 17.5 |
| | Hz, 1H), 5.35 (d, J = 17.8 Hz, 1H), 5.19 - 5.04 (m, 1H), 4.36 - |
| | 4.23 (m, 2H), 3.80 (s, 1H), 2.31 (dd, J = 15.4, 9.1 Hz, 2H), 2.21 - |
| | 2.14 (m, 1H), 2.14 - 2.03 (m, 4H), 2.00 (s, 6H), 1.86 - 1.71 |
| | (m, 3H), 1.70 - 1.61 (m, 2H), 1.50 - 1.37 (m, 2H), 1.32 - 1.23 |
| | (m, 1H), 1.05 (d, J = 6.8 Hz, 3H), 0.90 (d, J = 6.5 Hz, 3H), 0.62 |
| | (s, 3H). |
| 1777 | ¹H NMR (400 MHZ, CDC13) 8 14.12 (s, 1H), 8.73 (t, J = 1.8 |
| | Hz, 1H), 8.11 (dt, J = 7.9, 1.4 Hz, 1H), 7.85 (dt, J = 7.7, 1.4 |
| | Hz, 1H), 7.79 - 7.70 (m, 1H), 7.67 (t, J = 7.8 Hz, 1H), 7.22 (t, |
| | J = 7.6 Hz, 1H), 7.05 (d, J = 7.6 Hz, 2H), 6.80 (d, J = 7.3 Hz, |
| | 1H), 6.68 (d, J = 9.1 Hz, 1H), 6.22 (s, 1H), 5.82 (d, J = 17.6 |
| | Hz, 1H), 5.36 (d, J = 17.5 Hz, 1H), 5.10 (d, J = 6.3 Hz, 1H), |
| | 4.41 - 4.24 (m, 2H), 3.86 - 3.62 (m, 1H), 2.34 (dd, J = 15.3, 8.7 |
| | Hz, 1H), 2.27 - 2.14 (m, 3H), 2.00 (s, 6H), 1.91 - 1.85 (m, 1H), |
| | 1.82 - 1.71 (m, 3H), 1.71 - 1.61 (m, 3H), 1.49 - 1.40 (m, 2H), |
| | 1.36 (d, J = 6.3 Hz, 3H), 1.32 - 1.24 (m, 1H), 0.61 (s, 3H). |
| 1778 | ¹H NMR (400 MHZ, CDC13) 8 8.69 (t, J = 1.8 Hz, 1H), 7.92 (s, |
| | 1H), 7.85 - 7.76 (m, 2H), 7.67 (s, 1H), 7.56 (t, J = 7.7 Hz, 1H), |
| | 7.22 (t, J = 7.6 Hz, 1H), 7.05 (d, J = 7.6 Hz, 2H), 6.14 (s, 1H), |
| | 5.41 (dd, J = 10.3, 3.1 Hz, 1H), 5.16 (d, J = 15.3 Hz, 1H), 4.54 - |
| | 4.44 (m, 1H), 4.20 - 4.05 (m, 3H), 4.03 - 3.94 (m, 2H), 2.60 - |
| | 2.49 (m, 1H), 2.16 - 2.06 (m, 1H), 2.02 (s, 6H), 1.90 - 1.67 (m, |
| | 1H), 1.55 (d, J = 6.2 Hz, 4H), 0.55 - 0.42 (m, 2H), 0.31 - 0.20 |
| | (m, 1H), 0.11 - 0.03 (m, 1H), -0.39 (dq, J = 9.7, 5.0 Hz, 1H). |
| 1779 | ¹H NMR (400 MHZ, Chloroform-d) 8 8.82 (t, J = 1.8 Hz, 1H), |
| | 8.04 (d, J = 7.9 Hz, 1H), 7.84 (d, J = 8.6 Hz, 3H), 7.65 (t, J = |
| | 7.8 Hz, 1H), 7.23 (t, J = 7.6 Hz, 1H), 7.08 (d, J = 7.6 Hz, 2H), |
| | 6.24 (s, 1H), 5.52 (dd, J = 11.6, 4.1 Hz, 1H), 5.17 (d, J = 15.7 |
| | Hz, 1H), 4.55 - 4.44 (m, 1H), 4.38 (t, J = 11.6 Hz, 1H), 4.29 - |
| | 4.18 (m, 1H), 4.12 (d, J = 15.7 Hz, 1H), 3.20 (s, 3H), 2.40 - |
| | 2.28 (m, 2H), 2.23 (q, J = 9.8, 9.4 Hz, 2H), 2.05 (s, 6H), 1.81 - |
| | 1.72 (m, 2H), 1.62 (s, 1H), 1.58 - 1.51 (m, 1H), 1.47 - 1.37 (m, |
| | 2H), 1.17 (dt, J = 14.4, 7.5 Hz, 1H), 0.76 (t, J = 7.2 Hz, 3H), |
| | 0.46 (t, J = 7.4 Hz, 3H). Sulfonamide NH not visible. |
| 1780 | ¹H NMR (400 MHZ, Chloroform-d) 8 8.82 (s, 1H), 8.11 (d, J = |
| | 7.9 Hz, 1H), 7.87 (d, J = 7.2 Hz, 2H), 7.80 (s, 1H), 7.68 (t, J = |
| | 7.8 Hz, 1H), 7.23 (d, J = 7.6 Hz, 1H), 7.09 (d, J = 7.6 Hz, 2H), |
| | 6.31 (s, 1H), 5.48 (d, J = 7.4 Hz, 1H), 5.05 (d, J = 14.8 Hz, |
| | 1H), 4.37 - 4.30 (m, 1H), 4.23 (d, J = 15.0 Hz, 1H), 4.08 (t, J = |
| | 11.2 Hz, 1H), 3.85 - 3.74 (m, 1H), 3.72 - 3.65 (m, 1H), 3.61 - |
| | 3.51 (m, 1H), 3.39 (dd, J = 14.7, 6.6 Hz, 1H), 2.10 (s, 6H), 1.65 |
| | (d, J = 8.6 Hz, 1H), 1.57 (d, J = 15.2 Hz, 1H), 1.26 (d, J = 5.5 |
| | Hz, 3H), 0.91 - 0.86 (m, 1H), 0.61 (s, 9H), 0.59 - 0.51 (m, 2H), |
| | 0.34 - 0.25 (m, 2H). Sulfonamide NH not visible. |
| 1784 | ¹H NMR (400 MHZ, Chloroform-d) 8 8.87 (d, J = 5.3 Hz, 1H), |
| | 8.05 (d, J = 7.9 Hz, 1H), 7.92 (s, 1H), 7.87 (d, 1H), 7.76 (d, J = |
| | 9.1 Hz, 1H), 7.64 (t, J = 7.7 Hz, 1H), 7.21 (t, J = 7.6 Hz, 1H), |
| | 7.07 (d, J = 7.7 Hz, 2H), 6.26 (d, J = 3.3 Hz, 1H), 5.52 - 5.41 |
| | (m, 1H), 5.14 - 5.03 (m, 1H), 4.73 (dtd, J = 22.8, 7.0, 2.7 Hz, |
| | 1H), 4.28 - 4.19 (m, 1H), 4.14 - 4.02 (m, 2H), 3.49 (s, 1H), 3.18 |
| | (s, 2H), 3.14 (d, J = 15.2 Hz, 1H), 2.96 - 2.83 (m, 1H), 2.80 - |
| | 2.73 (m, 1H), 2.67 - 2.57 (m, 1H), 2.53 - 2.44 (m, 2H), 2.09 - |
| | 2.06 (m, 1H), 2.02 (s, 6H), 1.74 - 1.68 (m, 1H), 1.55 (d, J = |
| | 15.1 Hz, 2H), 0.62 (s, 9H). |
| 1785 | ¹H NMR (400 MHZ, Chloroform-d) 8 8.87 (s, 1H), 8.04 (d, J = |
| | 5.8 Hz, 1H), 7.96 (d, J = 1.9 Hz, 1H), 7.92 - 7.84 (m, 2H), 7.64 |
| | (t, J = 7.8 Hz, 1H), 7.21 (t, J = 7.6 Hz, 1H), 7.07 (d, J = 7.6 |
| | Hz, 2H), 6.26 (t, J = 1.1 Hz, 1H), 5.46 (ddd, J = 12.4, 8.7, 4.2 |
| | Hz, 1H), 5.07 (d, J = 15.5 Hz, 1H), 4.24 (d, J = 8.0 Hz, 1H), |
| | 4.14 - 4.04 (m, 3H), 3.15 (d, J = 1.7 Hz, 3H), 2.60 (s, 1H), 2.19 |
| | (qd, J = 8.6, 7.8, 3.7 Hz, 1H), 2.02 (d, J = 32.5 Hz, 6H), 1.97 - |
| | 1.87 (m, 2H), 1.71 (ddd, J = 15.2, 8.8, 2.4 Hz, 1H), 1.54 (d, J = |
| | 15.0 Hz, 1H), 1.38 - 1.26 (m, 1H), 1.13 (dd, J = 8.0, 6.5 Hz, |
| | 3H), 0.61 (s, 9H). |
| 1586 | ¹H NMR (400 MHZ, CDC13) 8 8.70 (s, 1H), 7.96 (d, J = 13.8 |
| | Hz, 2H), 7.92 - 7.85 (m, 1H), 7.81 (d, J = 7.6 Hz, 1H), 7.59 (t, |
| | J = 7.8 Hz, 1H), 7.22 (t, J = 7.6 Hz, 1H), 7.06 (d, J = 7.6 Hz, |
| | 2H), 6.17 (s, 1H), 5.45 - 5.38 (m, 1H), 5.17 (d, J = 15.4 Hz, |
| | 1H), 4.58 - 4.49 (m, 1H), 4.19 - 3.97 (m, 5H), 3.76 - 3.69 (m, |
| | 2H), 3.40 (s, 3H), 2.53 - 2.42 (m, 1H), 2.38 - 2.27 (m, 1H), 2.03 |
| | (s, 6H), 1.91 - 1.77 (m, 2H), 0.54 - 0.43 (m, 2H), 0.31 - 0.21 |
| | (m, 1H), 0.12 - 0.03 (m, 1H), -0.34 - - 0.44 (m, 1H). |
| 1787 | ¹H NMR (400 MHZ, CDC13) 8 8.80 - 8.78 (m, 1H), 7.95 (d, J = |
| | 7.9 Hz, 1H), 7.88 - 7.82 (m, 2H), 7.71 (s, 1H), 7.63 (t, J = 7.8 |
| | Hz, 1H), 7.23 (t, J = 7.6 Hz, 1H), 7.08 (d, J = 7.7 Hz, 2H), 6.21 |
| | (s, 1H), 5.48 (dd, J = 11.1, 4.0 Hz, 1H), 5.17 (d, J = 15.7 Hz, |
| | 1H), 4.29 - 4.20 (m, 2H), 4.15 - 4.05 (m, 2H), 3.72 - 3.65 (m, |
| | 1H), 3.56 - 3.48 (m, 1H), 2.18 - 2.10 (m, 3H), 2.06 (s, 6H), 1.88 - |
| | 1.78 (m, 2H), 1.34 - 1.26 (m, 4H), 0.55 - 0.43 (m, 2H), 0.30 - |
| | 0.22 (m, 1H), 0.12 - 0.04 (m, 1H), -0.37 - - 0.45 (m, 1H). |
| 1788 | ¹H NMR (400 MHZ, CDC13) 8 8.73 (t, J = 1.8 Hz, 1H), 8.00 (d, |
| | J = 7.9 Hz, 1H), 7.93 (s, 1H), 7.86 (s, 1H), 7.84 (dt, J = 7.6, 1.4 |
| | Hz, 1H), 7.64 (t, J = 7.8 Hz, 1H), 7.21 (t, J = 7.6 Hz, 1H), 7.04 |
| | (d, J = 7.6 Hz, 2H), 6.19 (s, 1H), 5.44 - 5.33 (m, 1H), 5.14 (d, |
| | J = 15.5 Hz, 1H), 4.72 (hept, J = 6.7 Hz, 1H), 4.13 - 3.93 (m, |
| | 3H), 2.96 (s, 3H), 2.01 (s, 6H), 1.70 - 1.54 (m, 2H), 1.26 (d, J = |
| | 6.6 Hz, 3H), 1.20 (d, J = 6.6 Hz, 3H), 1.18 - 1.11 (m, 1H), |
| | 0.81 (s, 9H), 0.80 - 0.73 (m, 1H). |
| 1789 | ¹H NMR (400 MHZ, CDC13) 8 8.71 (s, 1H), 7.98 (d, J = 7.9 Hz, |
| | 1H), 7.74 (d, J = 7.6 Hz, 1H), 7.46 (s, 1H), 7.41 - 7.30 (m, 1H), |
| | 7.13 (t, J = 7.6 Hz, 1H), 6.96 (d, J = 7.6 Hz, 2H), 6.63 (d, J = |
| | 7.2 Hz, 1H), 6.24 (d, J = 8.4 Hz, 1H), 6.06 (s, 1H), 5.35 (dd, J = |
| | 10.9, 3.6 Hz, 1H), 5.22 (d, J = 15.1 Hz, 1H), 4.15 (p, J = 6.6 |
| | Hz, 1H), 4.10 - 3.92 (m, 3H), 3.63 - 3.55 (m, 1H), 3.47 - 3.37 |
| | (m, 1H), 2.11 - 2.02 (m, 2H), 1.94 (s, 6H), 1.74 - 1.66 (m, 2H), |
| | 1.60 - 1.51 (m, 1H), 1.24 (d, J = 6.3 Hz, 3H), 1.17 (td, J = 12.6, |
| | 4.0 Hz, 2H), 0.82 (s, 9H), 0.78 - 0.73 (m, 1H). |
| 1790 | ¹H NMR (400 MHZ, CDC13) & 8.67 (t, J = 1.7 Hz, 1H), 8.05 (d, |
| | J = 7.9 Hz, 1H), 7.77 (d, J = 7.5 Hz, 1H), 7.53 (t, J = 7.8 Hz, |
| | 1H), 7.41 (dd, J = 8.5, 7.2 Hz, 1H), 7.15 (t, J = 7.6 Hz, 1H), |
| | 6.96 (d, J = 7.6 Hz, 2H), 6.65 (d, J = 7.2 Hz, 1H), 6.39 (d, J = |
| | 8.6 Hz, 1H), 6.06 (s, 1H), 5.34 - 5.17 (m, 2H), 4.82 (h, J = 6.7 |
| | Hz, 1H), 4.07 - 3.87 (m, 3H), 2.89 (s, 3H), 1.94 (s, 6H), 1.79 - |
| | 1.64 (m, 1H), 1.61 - 1.45 (m, 1H), 1.21 (d, J = 6.7 Hz, 3H), |
| | 1.17 (d, J = 6.6 Hz, 3H), 1.16 - 1.11 (m, 1H), 0.82 (s, 9H), 0.76 |
| | (dd, J = 13.0, 4.4 Hz, 1H). |
| 1791 | ¹H NMR (400 MHZ, CDC13) 8 8.72 (t, J = 1.8 Hz, 1H), 8.04 (d, |
| | J = 7.9 Hz, 1H), 7.91 (s, 1H), 7.87 (s, 1H), 7.83 (dt, J = 7.7, 1.4 |
| | Hz, 1H), 7.65 (t, J = 7.8 Hz, 1H), 7.20 (t, J = 7.6 Hz, 1H), 7.03 |
| | (d, J = 7.6 Hz, 2H), 6.17 (s, 1H), 5.44 - 5.32 (m, 1H), 5.18 (d, |
| | J = 15.4 Hz, 1H), 4.09 - 4.00 (m, 2H), 3.97 (d, J = 15.4 Hz, |
| | 1H), 3.42 - 3.29 (m, 2H), 3.18 (s, 3H), 2.16 - 2.03 (m, 1H), 1.99 |
| | (s, 6H), 1.67 - 1.55 (m, 2H), 1.16 (td, J = 12.4, 5.3 Hz, 1H), |
| | 0.93 (d, J = 6.7 Hz, 3H), 0.91 (d, J = 6.8 Hz, 3H), 0.82 (s, 9H), |
| | 0.81 - 0.75 (m, 1H). |
| 1792 | ¹H NMR (400 MHZ, CDC13) 8 8.74 - 8.71 (m, 1H), 8.00 (d, J = |
| | 7.5 Hz, 2H), 7.91 - 7.83 (m, 2H), 7.64 (t, J = 7.8 Hz, 1H), 7.21 |
| | (t, J = 7.6 Hz, 1H), 7.06 (d, J = 7.6 Hz, 2H), 6.20 (s, 1H), 5.34 |
| | (dd, J = 11.1, 4.2 Hz, 1H), 5.20 (d, J = 15.5 Hz, 1H), 4.71 - |
| | 4.59 (m, 1H), 4.30 - 4.17 (m, 2H), 4.11 - 3.99 (m, 2H), 3.02 (td, |
| | J = 12.9, 3.1 Hz, 1H), 2.04 (s, 6H), 1.93 - 1.73 (m, 5H), 1.73 - |
| | 1.59 (m, 6H), 0.53 - 0.44 (m, 2H), 0.29 - 0.19 (m, 1H), 0.10 - |
| | 0.03 (m, 1H), -0.41 - - 0.49 (m, 1H). |
| 1797 | ¹H NMR (400 MHZ, Chloroform-d) 8 8.77 (s, 1H), 8.06 (d, J = |
| | 7.9 Hz, 1H), 7.94 (s, 1H), 7.85 (d, J = 7.6 Hz, 1H), 7.71 (s, 1H), |
| | 7.66 (t, J = 7.7 Hz, 1H), 7.21 (t, J = 7.6 Hz, 1H), 7.05 (d, J = |
| | 7.6 Hz, 2H), 6.22 (s, 1H), 5.40 (d, J = 7.8 Hz, 1H), 5.16 (d, J = |
| | 15.5 Hz, 1H), 4.25 (q, J = 6.5 Hz, 1H), 4.15 - 3.93 (m, 5H), |
| | 2.36 - 2.28 (m, 1H), 2.27 - 2.16 (m, 2H), 2.06 - 1.99 (m, 6H), |
| | 1.63 - 1.57 (m, 3H), 1.17 (s, 1H), 1.00 (d, J = 6.7, 1.2 Hz, 3H), |
| | 0.90 (d, J = 6.8 Hz, 3H), 0.81 (s, 9H). Sulfonamide NH not |
| | visible. |
| 1798 | ¹H NMR (400 MHZ, Chloroform-d) 8 8.71 (t, J = 1.7 Hz, 1H), |
| | 8.05 (d, J = 7.9 Hz, 1H), 7.97 (s, 1H), 7.85 (d, J = 7.5 Hz, 1H), |
| | 7.71 (s, 1H), 7.66 (t, J = 7.8 Hz, 1H), 7.21 (t, J = 7.6 Hz, 1H), |
| | 7.06 (d, J = 7.6 Hz, 2H), 6.24 (s, 1H), 5.41 (dd, J = 11.3, 4.1 |
| | Hz, 1H), 5.10 (d, J = 15.1 Hz, 1H), 4.31 (dt, J = 8.5, 5.8 Hz, |
| | 1H), 4.17 (t, J = 11.4 Hz, 1H), 4.11 - 3.94 (m, 4H), 2.38 - 2.22 |
| | (m, 3H), 2.04 (s, 6H), 1.65 - 1.61 (m, 2H), 1.19 - 1.13 (m, 2H), |
| | 1.02 (d, J = 6.7 Hz, 3H), 0.91 (d, J = 6.8 Hz, 3H), 0.81 (s, 9H). |
| | Sulfonamide NH not visible. |
| 1799 | ¹H NMR (400 MHZ, Chloroform-d) 8 8.88 (s, 1H), 8.06 (d, J = |
| | 7.9 Hz, 1H), 7.94 (s, 1H), 7.87 (d, J = 7.6 Hz, 1H), 7.70 (s, 1H), |
| | 7.65 (t, J = 7.8 Hz, 1H), 7.21 (t, J = 7.6 Hz, 1H), 7.07 (d, J = |
| | 7.6 Hz, 2H), 6.26 (d, J = 1.0 Hz, 1H), 5.44 (dd, J = 11.4, 4.3 |
| | Hz, 1H), 5.08 (d, J = 15.5 Hz, 1H), 4.43 (dt, J = 8.5, 4.4 Hz, |
| | 1H), 4.26 - 4.17 (m, 1H), 4.12 - 3.97 (m, 3H), 3.83 (dd, J = 8.0, |
| | 3.8 Hz, 1H), 2.73 - 2.63 (m, 1H), 2.02 (s, 6H), 1.96 - 1.89 (m, |
| | 1H), 1.80 - 1.63 (m, 6H), 1.53 - 1.46 (m, 2H), 1.41 - 1.35 (m, |
| | 1H), 0.61 (s, 9H). Sulfonamide NH not visible. |
| 1800 | ¹H NMR (400 MHZ, Chloroform-d) 8 8.84 (t, J = 1.8 Hz, 1H), |
| | 8.05 (d, J = 7.8 Hz, 1H), 7.96 (s, 1H), 7.89 - 7.83 (m, 1H), 7.69 |
| | (s, 1H), 7.64 (t, J = 7.8 Hz, 1H), 7.21 (t, J = 7.6 Hz, 1H), 7.08 |
| | (d, J = 7.7 Hz, 2H), 6.27 (s, 1H), 5.51 (dd, J = 11.0, 4.0 Hz, |
| | 1H), 4.99 (d, J = 15.3 Hz, 1H), 4.44 (dt, J = 8.5, 4.4 Hz, 1H), |
| | 4.25 - 4.16 (m, 1H), 4.15 - 4.03 (m, 3H), 3.83 (dd, J = 8.0, 3.9 |
| | Hz, 1H), 2.71 (dt, J = 11.0, 5.3 Hz, 1H), 2.08 (dt, J = 9.6, 5.1 |
| | Hz, 1H), 2.06 - 1.99 (m, 6H), 1.95 - 1.74 (m, 3H), 1.73 - 1.65 |
| | (m, 3H), 1.53 - 1.48 (m, 2H), 1.43 - 1.36 (m, 1H), 0.61 (s, 9H). |
| | Sulfonamide NH not visible. |
| 1801 | ¹H NMR (400 MHZ, CDC13) 8 8.74 (d, J = 1.8 Hz, 1H), 7.93 |
| | (s, 1H), 7.83 - 7.77 (m, 2H), 7.66 (s, 1H), 7.56 (t, J = 7.8 Hz, |
| | 1H), 7.23 (t, J = 7.6 Hz, 1H), 7.08 (d, J = 7.6 Hz, 2H), 6.23 (s, |
| | 1H), 5.49 (dd, J = 11.4, 4.5 Hz, 1H), 5.04 (d, J = 15.3 Hz, 1H), |
| | 4.50 (dt, J = 8.1, 6.1 Hz, 1H), 4.44 - 4.35 (m, 1H), 4.18 - 4.08 |
| | (m, 2H), 4.04 - 3.94 (m, 2H), 2.61 - 2.49 (m, 1H), 2.04 (s, 6H), |
| | 1.94 - 1.80 (m, 2H), 1.55 (d, J = 6.1 Hz, 3H), 1.24 - 1.16 (m, |
| | 4H), 0.98 - 0.90 (m, 3H). |
| 1802 | ¹H NMR (400 MHZ, CDC13) 8 8.77 - 8.73 (m, 1H), 7.96 (d, J = |
| | 13.1 Hz, 2H), 7.84 (dd, J = 17.8, 7.8 Hz, 2H), 7.58 (t, J = 7.8 |
| | Hz, 1H), 7.23 (t, J = 7.6 Hz, 1H), 7.08 (d, J = 7.6 Hz, 2H), 6.24 |
| | (s, 1H), 5.50 (dd, J = 11.4, 4.5 Hz, 1H), 5.07 (d, J = 15.4 Hz, |
| | 1H), 4.58 - 4.50 (m, 1H), 4.45 - 4.35 (m, 1H), 4.21 - 3.99 (m, |
| | 4H), 3.76 - 3.67 (m, 2H), 3.40 (s, 3H), 2.52 - 2.42 (m, 1H), 2.37 - |
| | 2.27 (m, 1H), 2.04 (s, 6H), 1.24 - 1.17 (m, 4H), 0.98 - 0.91 |
| | (m, 4H). |
| 1803 | ¹H NMR (400 MHZ, CDC13) 8 8.80 (s, 1H), 7.91 - 7.68 (m, 4H), |
| | 7.54 (t, J = 7.8 Hz, 1H), 7.26 - 7.20 (m, 1H), 7.08 (d, J = 7.6 |
| | Hz, 2H), 6.21 (s, 1H), 5.51 (dd, J = 11.4, 4.5 Hz, 1H), 5.09 (d, |
| | J = 15.4 Hz, 1H), 4.47 - 4.36 (m, 1H), 4.26 - 4.16 (m, 1H), 4.14 - |
| | 3.98 (m, 2H), 3.68 - 3.60 (m, 1H), 3.51 - 3.41 (m, 1H), 2.04 |
| | (s, 7H), 1.94 - 1.82 (m, 2H), 1.80 - 1.72 (m, 1H), 1.28 - 1.20 |
| | (m, 7H), 1.18 (s, 2H), 0.98 (s, 1H), 0.93 (s, 1H). |
| 1804 | ¹H NMR (400 MHZ, Chloroform-d) 8 8.78 (d, J = 1.8 Hz, 1H), |
| | 8.07 (d, J = 7.9 Hz, 1H), 7.85 (dd, J = 17.6, 10.1 Hz, 3H), 7.65 |
| | (t, J = 7.8 Hz, 1H), 7.22 (t, J = 7.6 Hz, 1H), 7.07 (d, J = 7.6 |
| | Hz, 2H), 6.25 (s, 1H), 5.42 (dd, J = 11.0, 4.2 Hz, 1H), 5.13 (d, |
| | J = 15.6 Hz, 1H), 4.61 - 4.47 (m, 1H), 4.24 (t, J = 11.0 Hz, 1H), |
| | 4.13 (d, J = 15.6 Hz, 1H), 4.05 (t, J = 11.2 Hz, 1H), 3.18 (s, |
| | 3H), 2.38 - 2.27 (m, 2H), 2.23 (q, J = 9.6 Hz, 2H), 2.04 (s, 6H), |
| | 1.76 (td, J = 10.7, 4.9 Hz, 2H), 1.63 (t, J = 13.1 Hz, 3H), 1.53 - |
| | 1.41 (m, 3H), 1.20 - 1.08 (m, 2H), 1.05 - 0.97 (m, 2H), 0.92 - |
| | 0.83 (m, 3H). Sulfonamide NH not visible. |
| 1805 | ¹H NMR (400 MHZ, CDC13) 8 8.74 (s, 1H), 8.00 (s, 1H), 7.91 - |
| | 7.80 (m, 3H), 7.59 (t, J = 7.8 Hz, 1H), 7.22 (t, J = 7.6 Hz, 1H), |
| | 7.07 (d, J = 7.6 Hz, 2H), 6.23 (s, 1H), 5.39 (dd, J = 11.3, 4.6 |
| | Hz, 1H), 5.09 (d, J = 15.5 Hz, 1H), 4.70 - 4.59 (m, 1H), 4.49 - |
| | 4.39 (m, 1H), 4.30 - 4.19 (m, 1H), 4.13 - 3.98 (m, 2H), 3.07 - |
| | 2.93 (m, 1H), 2.06 (s, 6H), 1.93 - 1.79 (m, 3H), 1.72 - 1.62 (m, |
| | 4H), 1.28 - 1.20 (m, 8H), 1.16 (s, 2H). |
| 1324 | ¹H NMR (400 MHZ, DMSO-d6) 8 8.59 (s, 1H), 7.99 (s, 1H), |
| | 7.95 - 7.89 (m, 1H), 7.87 (s, 1H), 7.74 - 7.59 (m, 2H), 7.26 (t, J = |
| | 7.7 Hz, 1H), 7.12 (d, J = 7.6 Hz, 2H), 6.36 (br. s., 1H), 5.32 |
| | (dd, J = 11.0, 4.4 Hz, 1H), 4.83 - 4.73 (m, 2H), 4.49 (d, J = |
| | 16.1 Hz, 1H), 4.42 (t, J = 11.5 Hz, 1H), 4.25 - 4.14 (m, 1H), |
| | 3.70 (dd, J = 10.9, 8.9 Hz, 1H), 3.46 (dd, J = 11.0, 3.4 Hz, 1H), |
| | 3.37 - 3.33 (m, 1H, overlapped with water), 3.05 (s, 3H), 2.24 - |
| | 2.12 (m, J = 8.8, 8.8 Hz, 4H), 2.06 (br. s., 6H), 1.70 - 1.58 (m, |
| | 2H), 0.98 (d, J = 5.9 Hz, 3H), 0.89 (d, J = 6.1 Hz, 3H), (1H |
| | missing, labile proton). |
| 1806 | ¹H NMR (400 MHZ, CDC13) 8 8.77 (d, J = 1.8 Hz, 1H), 8.06 |
| | (d, J = 7.9 Hz, 1H), 7.91 (d, J = 1.8 Hz, 2H), 7.85 (d, J = 7.4 |
| | Hz, 1H), 7.67 (t, J = 7.7 Hz, 1H), 7.21 (t, J = 7.6 Hz, 1H), 7.05 |
| | (d, J = 7.6 Hz, 2H), 6.23 (s, 1H), 5.43 (d, J = 7.6 Hz, 1H), 5.16 |
| | (d, J = 15.5 Hz, 1H), 4.08 - 3.97 (m, 3H), 3.20 (s, 6H), 2.03 (s, |
| | 6H), 1.66 - 1.59 (m, 3H), 1.17 (dt, J = 13.6, 8.2 Hz, 1H), 0.82 |
| | (s, 9H). |
| 1813 | ¹H NMR (400 MHZ, CDC13) 8 8.80 (s, 1H), 8.06 (d, J = 7.9 Hz, |
| | 1H), 7.91 - 7.84 (m, 3H), 7.67 (t, J = 7.8 Hz, 1H), 7.21 (t, J = |
| | 7.6 Hz, 1H), 7.07 (d, J = 7.6 Hz, 2H), 6.27 (s, 1H), 5.53 - 5.46 |
| | (m, 1H), 5.12 (d, J = 15.5 Hz, 1H), 4.16 - 3.91 (m, 4H), 3.74 - |
| | 3.45 (m, 2H), 2.10 - 1.97 (m, 10H), 1.22 - 1.10 (m, 1H), 1.06 - |
| | 0.96 (m, 1H), 0.84 - 0.71 (m, 12H), 0.64 - 0.36 (m, 3H), 0.25 - |
| | 0.12 (m, 1H). |
| 1814 | ¹H NMR (400 MHZ, CDC13) 8 8.81 - 8.78 (m, 1H), 8.05 - 7.99 |
| | (m, 1H), 7.89 - 7.83 (m, 2H), 7.74 (s, 1H), 7.66 (t, J = 7.8 Hz, |
| | 1H), 7.22 (t, J = 7.6 Hz, 1H), 7.07 (d, J = 7.6 Hz, 2H), 6.25 (s, |
| | 1H), 5.51 - 5.45 (m, 1H), 5.11 (d, J = 15.5 Hz, 1H), 4.25 - 4.18 |
| | (m, 1H), 4.15 - 4.00 (m, 3H), 3.70 - 3.62 (m, 1H), 3.53 - 3.43 |
| | (m, 1H), 2.16 - 2.02 (m, 10H), 1.68 - 1.58 (m, 3H), 1.28 (d, J = |
| | 6.3 Hz, 3H), 0.83 - 0.79 (m, 10H). |
| 1815 | ¹H NMR (400 MHZ, CDC13) 8 8.72 - 8.69 (m, 1H), 8.14 (d, J = |
| | 7.9 Hz, 1H), 8.04 (s, 1H), 7.96 (s, 1H), 7.88 - 7.83 (m, 1H), 7.69 |
| | (t, J = 7.8 Hz, 1H), 7.20 (t, J = 7.6 Hz, 1H), 7.03 (d, J = 7.7 |
| | Hz, 2H), 6.20 (s, 1H), 5.35 (d, J = 6.8 Hz, 1H), 5.20 (d, J = |
| | 15.5 Hz, 1H), 4.80 - 4.69 (m, 1H), 4.09 - 3.97 (m, 3H), 3.13 (s, |
| | 3H), 3.08 - 2.93 (m, 2H), 2.84 - 2.68 (m, 2H), 2.62 (s, 1H), 2.01 |
| | (s, 6H), 1.68 - 1.57 (m, 2H), 1.21 - 1.10 (m, 1H), 0.82 (s, 9H). |
| 1816 | ¹H NMR (400 MHZ, CDC13) 8 8.77 (s, 1H), 8.08 - 8.01 (m, 1H), |
| | 7.90 - 7.82 (m, 2H), 7.75 - 7.70 (m, 1H), 7.70 - 7.62 (m, 1H), |
| | 7.24 - 7.17 (m, 1H), 7.10 - 7.03 (m, 2H), 6.28 - 6.24 (m, 1H), |
| | 5.48 - 5.40 (m, 1H), 5.13 (d, J = 15.3 Hz, 1H), 4.17 - 3.90 (m, |
| | 5H), 3.69 - 3.61 (m, 1H), 3.58 - 3.48 (m, 1H), 2.04 (s, 10H), |
| | 0.95 - 0.88 (m, 4H), 0.87 - 0.70 (m, 13H). |
| 1817 | ¹H NMR (400 MHZ, CDC13) 8 8.79 - 8.75 (m, 1H), 8.12 - 8.07 |
| | (m, 1H), 7.92 (d, J = 5.6 Hz, 2H), 7.89 - 7.84 (m, 1H), 7.68 (t, |
| | J = 7.7 Hz, 1H), 7.21 (t, J = 7.6 Hz, 1H), 7.06 (d, J = 7.6 Hz, |
| | 2H), 6.25 (s, 1H), 5.46 - 5.40 (m, 1H), 5.16 (d, J = 15.5 Hz, |
| | 1H), 4.94 - 4.85 (m, 1H), 4.12 - 4.00 (m, 3H), 3.22 (s, 3H), 2.59 - |
| | 2.45 (m, 2H), 2.33 - 2.25 (m, 2H), 2.04 (s, 6H), 1.65 - 1.61 |
| | (m, 3H), 1.16 (d, J = 8.3 Hz, 1H), 0.82 (s, 9H), 0.51 - 0.42 (m, |
| | 4H). |
| 1818 | 'H NMR (400 MHZ, CDC13) 8 8.73 (s, 1H), 8.13 (d, J = 7.9 Hz, |
| | 1H), 8.05 - 7.90 (m, 2H), 7.85 (d, J = 7.6 Hz, 1H), 7.68 (t, J = |
| | 7.8 Hz, 1H), 7.19 (t, J = 7.6 Hz, 1H), 7.03 (d, J = 7.6 Hz, 2H), |
| | 6.18 (s, 1H), 5.34 - 5.27 (m, 1H), 5.24 (d, J = 15.6 Hz, 1H), |
| | 4.09 - 3.89 (m, 4H), 3.79 - 3.65 (m, 1H), 3.21 (s, 3H), 1.99 (s, |
| | 6H), 1.64 - 1.57 (m, 3H), 1.28 - 1.24 (m, 9H), 0.82 (s, 7H). |
| 1819 | ¹H NMR (400 MHZ, CDC13) 8 8.87 (s, 1H), 8.70 - 8.62 (m, 1H), |
| | 8.23 (d, J = 7.9 Hz, 1H), 7.94 (d, J = 7.6 Hz, 1H), 7.77 (t, J = |
| | 7.8 Hz, 1H), 7.31 (t, J = 7.7 Hz, 1H), 7.14 (d, J = 7.7 Hz, 2H), |
| | 6.93 (s, 1H), 6.44 (s, 1H), 5.71 - 5.46 (m, 1H), 4.84 - 4.41 (m, |
| | 2H), 4.03 (s, 3H), 3.33 - 3.22 (m, 1H), 2.31 - 2.02 (m, 9H), 1.45 - |
| | 1.40 (m, 6H), 1.26 - 1.08 (m, 7H). |
| 1820 | ¹H NMR (400 MHZ, CDC13) 8 8.88 (s, 1H), 8.56 - 8.48 (m, 1H), |
| | 8.06 (d, J = 7.9 Hz, 1H), 7.86 (d, J = 7.6 Hz, 1H), 7.67 (t, J = |
| | 7.7 Hz, 1H), 7.23 (t, J = 7.6 Hz, 2H), 7.08 (d, J = 7.6 Hz, 2H), |
| | 6.24 (s, 1H), 5.60 - 5.44 (m, 1H), 4.33 - 4.07 (m, 1H), 4.01 (s, |
| | 3H), 3.32 - 3.16 (m, 1H), 2.62 (s, 3H), 2.10 - 2.02 (m, 8H), 1.42 |
| | (t, J = 7.2 Hz, 6H), 0.55 (s, 2H), 0.31 (s, 1H), 0.20 - 0.09 (m, |
| | 1H), -0.30 - - 0.40 (m, 1H). |
| 1821 | ¹H NMR (400 MHZ, Chloroform-d) 8 8.77 (d, J = 1.8 Hz, 1H), |
| | 8.06 (d, J = 7.9 Hz, 1H), 7.89 (s, 2H), 7.84 (d, J = 7.6 Hz, 1H), |
| | 7.64 (t, J = 7.8 Hz, 1H), 7.21 (t, J = 7.6 Hz, 1H), 7.07 (d, J = |
| | 7.6 Hz, 2H), 6.24 (s, 1H), 5.41 (dd, J = 11.1, 4.2 Hz, 1H), 5.11 |
| | (d, J = 15.4 Hz, 1H), 4.22 (t, J = 11.1 Hz, 1H), 4.13 - 4.01 (m, |
| | 2H), 3.35 (d, J = 7.5 Hz, 2H), 3.20 (s, 3H), 2.15 - 2.07 (m, 1H), |
| | 2.04 (s, 6H), 1.64 - 1.62 (m, 2H), 1.53 - 1.49 (m, 2H), 1.46 - |
| | 1.37 (m, 3H), 1.23 - 1.16 (m, 1H), 1.14 - 1.08 (m, 1H), 1.04 - |
| | 0.96 (m, 2H), 0.92 (dd, J = 6.7, 3.9 Hz, 6H), 0.88 - 0.84 (m, |
| | 1H), 0.34 (q, J = 11.8 Hz, 1H). (Sulfonamide NH not visible) |
| 1822 | ¹H NMR (400 MHZ, Chloroform-d) 8 8.74 (d, J = 2.3 Hz, 1H), |
| | 8.04 (d, J = 7.9 Hz, 1H), 7.99 (s, 1H), 7.87 (s, 1H), 7.83 (d, J = |
| | 7.6 Hz, 1H), 7.63 (t, J = 7.8, 2.0 Hz, 1H), 7.21 (t, J = 7.6, 2.0 |
| | Hz, 1H), 7.07 (d, J = 7.6 Hz, 2H), 6.23 (d, J = 2.0 Hz, 1H), 5.39 |
| | - 5.30 (m, 1H), 5.10 (dd, J = 15.6, 2.0 Hz, 1H), 4.65 (d, J = 7.8 |
| | Hz, 1H), 4.25 (d, J = 13.0 Hz, 2H), 4.11 - 4.00 (m, 2H), 3.00 (t, |
| | J = 12.0 Hz, 1H), 2.04 (s, 6H), 1.85 - 1.66 (m, 7H), 1.55 - 1.46 |
| | (m, 4H), 1.44 - 1.32 (m, 3H), 1.23 (dd, J = 6.8, 2.0 Hz, 3H), |
| | 1.04 - 0.95 (m, 2H), 0.90 - 0.80 (m, 2H), 0.32 (q, J = 11.6 Hz, |
| | 1H). (Sulfonamide -NH not visible) |
| 1824 | ¹H NMR (400 MHZ, Chloroform-d) 8 8.76 (d, J = 1.8 Hz, 1H), |
| | 8.05 (dt, J = 8.0, 1.4 Hz, 1H), 7.91 (s, 1H), 7.87 - 7.78 (m, 2H), |
| | 7.63 (t, J = 7.8 Hz, 1H), 7.21 (t, J = 7.6 Hz, 1H), 7.07 (d, J = |
| | 7.6 Hz, 2H), 6.25 (s, 1H), 5.44 (dd, J = 10.7, 3.7 Hz, 1H), 5.07 |
| | (d, J = 15.2 Hz, 1H), 4.23 (t, J = 11.1 Hz, 1H), 4.14 (t, J = 11.1 |
| | Hz, 1H), 4.10 - 4.01 (m, 2H), 3.66 - 3.53 (m, 2H), 2.35 (dq, J = |
| | 13.1, 6.8 Hz, 1H), 2.04 (s, 6H), 2.00 (q, J = 3.8 Hz, 1H), 1.98 - |
| | 1.91 (m, 2H), 1.65 (s, 2H), 1.50 (s, 2H), 1.46 - 1.34 (m, 3H), |
| | 1.23 - 1.13 (m, 1H), 1.13 - 1.05 (m, 1H), 1.01 (t, J = 10.2 Hz, |
| | 2H), 0.92 (d, J = 7.0 Hz, 3H), 0.87 (s, 2H), 0.82 (d, J = 6.8 Hz, |
| | 3H), 0.32 (q, J = 10.9, 10.5 Hz, 1H). (Sulfonamide -NH not |
| | visible). |
| 1825 | ¹H NMR (400 MHZ, Chloroform-d) 8 8.78 (t, J = 1.9 Hz, 1H), |
| | 8.06 (d, J = 7.9 Hz, 1H), 7.90 (d, J = 14.1 Hz, 2H), 7.83 (d, J = |
| | 8.0 Hz, 1H), 7.64 (t, J = 7.8 Hz, 1H), 7.21 (t, J = 7.6 Hz, 1H), |
| | 7.07 (d, J = 7.6 Hz, 2H), 6.24 (s, 1H), 5.41 (dd, J = 11.1, 4.3 |
| | Hz, 1H), 5.09 (d, J = 15.6 Hz, 1H), 4.74 (p, J = 6.7 Hz, 1H), |
| | 4.25 (t, J = 11.4 Hz, 1H), 4.13 - 4.02 (m, 2H), 2.97 (s, 3H), 2.05 |
| | (s, 6H), 1.52 - 1.48 (m, 2H), 1.48 - 1.34 (m, 4H), 1.26 (d, J = |
| | 6.6 Hz, 3H), 1.21 (d, J = 6.6 Hz, 3H), 1.18 - 1.05 (m, 2H), 1.00 |
| | (t, J = 10.0 Hz, 2H), 0.90 - 0.82 (m, 2H), 0.31 (q, J = 9.7 Hz, |
| | 1H). (Sulfonamide NH not visible) |
| 1826 | ¹H NMR (400 MHZ, Chloroform-d) 8 8.90 (d, J = 1.8 Hz, 1H), |
| | 8.49 (s, 1H), 8.04 (d, J = 7.9 Hz, 1H), 7.86 (dt, J = 7.6, 1.4 Hz, |
| | 1H), 7.66 (t, J = 7.8 Hz, 1H), 7.20 (t, J = 7.6 Hz, 1H), 7.04 (d, |
| | J = 7.7 Hz, 2H), 6.46 (s, 1H), 6.20 (s, 1H), 5.53 - 5.43 (m, 2H), |
| | 4.27 (d, J = 15.7 Hz, 1H), 4.08 - 3.97 (m, 2H), 3.90 (s, 3H), |
| | 3.16 (p, J = 6.8 Hz, 1H), 2.02 (s, 6H), 1.72 - 1.66 (m, 3H), 1.39 |
| | (dd, J = 10.0, 6.8 Hz, 6H), 1.28 - 1.20 (m, 1H), 0.85 (s, 9H). |
| | (Sulfonamide NH not visible). |
| 1827 | ¹H NMR (400 MHZ, Chloroform-d) 8 8.90 (t, J = 1.9 Hz, 1H), |
| | 8.49 (s, 1H), 7.96 (d, J = 7.9 Hz, 1H), 7.85 (dt, J = 7.6, 1.4 Hz, |
| | 1H), 7.64 (t, J = 7.7 Hz, 1H), 7.21 (t, J = 7.6 Hz, 1H), 7.04 (d, |
| | J = 7.6 Hz, 2H), 6.46 (s, 1H), 6.19 (s, 1H), 5.53 - 5.42 (m, 2H), |
| | 4.29 (d, J = 15.7 Hz, 1H), 4.12 - 3.99 (m, 2H), 3.90 (s, 3H), |
| | 3.16 (p, J = 6.8 Hz, 1H), 2.02 (s, 6H), 1.78 - 1.71 (m, 2H), 1.38 |
| | (dd, J = 9.8, 6.8 Hz, 6H), 1.36 - 1.30 (m, 1H), 1.24 - 1.14 (m, |
| | 1H), 0.99 - 0.91 (m, 1H), 0.82 (dd, J = 8.3, 6.6 Hz, 6H). |
| | Sulfonamide NH not visible. |
| 1828 | ¹H NMR (400 MHZ, Chloroform-d) 8 8.90 (t, J = 1.8 Hz, 1H), |
| | 8.49 (s, 1H), 7.93 (d, J = 7.9 Hz, 1H), 7.83 (dt, J = 7.6, 1.4 Hz, |
| | 1H), 7.62 (t, J = 7.8 Hz, 1H), 7.22 (t, J = 7.6 Hz, 1H), 7.06 (d, |
| | J = 7.6 Hz, 2H), 6.51 (s, 1H), 6.21 (s, 1H), 5.51 (dd, J = 10.8, |
| | 3.5 Hz, 1H), 5.45 (d, J = 15.7 Hz, 1H), 4.32 (d, J = 15.7 Hz, |
| | 1H), 4.21 - 4.03 (m, 2H), 3.91 (s, 3H), 3.17 (hept, J = 6.9 Hz, |
| | 1H), 2.04 (s, 6H), 1.89 - 1.82 (m, 2H), 1.39 (dd, J = 9.7, 6.8 Hz, |
| | 6H), 1.13 - 1.05 (m, 2H), 0.45 (p, J = 6.5, 5.6 Hz, 1H), 0.36 - |
| | 0.27 (m, 2H), -0.07 - - 0.12 (m, 2H). (Sulfonamide -NH not |
| | visible) |
| 1829 | ¹H NMR (400 MHZ, CDC13) 8 8.99 (t, J = 1.8 Hz, 1H), 8.49 (s, |
| | 1H), 8.00 (d, J = 7.9 Hz, 1H), 7.88 - 7.78 (m, 1H), 7.62 (t, J = |
| | 7.8 Hz, 1H), 7.22 (t, J = 7.6 Hz, 1H), 7.07 (d, J = 7.6 Hz, 2H), |
| | 6.48 (s, 1H), 6.24 (s, 1H), 5.56 (dd, J = 11.1, 4.1 Hz, 1H), 5.43 |
| | (d, J = 15.7 Hz, 1H), 4.42 (d, J = 15.8 Hz, 1H), 4.26 - 4.15 (m, |
| | 1H), 4.06 (t, J = 11.4 Hz, 1H), 3.91 (s, 3H), 3.16 (hept, J = 6.8 |
| | Hz, 1H), 2.02 (s, 6H), 1.97 - 1.92 (m, 1H), 1.91 - 1.79 (m, 2H), |
| | 1.75 - 1.66 (m, 2H), 1.61 - 1.53 (m, 1H), 1.49 - 1.41 (m, 2H), |
| | 1.39 (d, J = 6.8 Hz, 3H), 1.37 (d, J = 6.8 Hz, 3H), 0.71 (s, 3H). |
| 1830 | ¹H NMR (400 MHZ, CDC13) 8 8.90 (t, J = 1.8 Hz, 1H), 8.47 (s, |
| | 1H), 7.91 (d, J = 7.9 Hz, 1H), 7.86 - 7.80 (m, 1H), 7.60 (t, J = |
| | 7.8 Hz, 1H), 7.22 (t, J = 7.6 Hz, 1H), 7.05 (d, J = 7.6 Hz, 2H), |
| | 6.46 (s, 1H), 6.20 (s, 1H), 5.52 - 5.42 (m, 2H), 4.30 (d, J = 15.8 |
| | Hz, 1H), 4.07 - 3.97 (m, 2H), 3.90 (s, 3H), 3.15 (hept, J = 6.8 |
| | Hz, 1H), 2.19 (h, J = 7.7 Hz, 1H), 2.10 - 2.04 (m, 1H), 2.03 (s, |
| | 6H), 1.93 - 1.85 (m, 1H), 1.84 - 1.78 (m, 1H), 1.74 - 1.67 (m, |
| | 3H), 1.66 - 1.58 (m, 1H), 1.39 (d, J = 6.8 Hz, 3H), 1.37 (d, J = |
| | 6.8 Hz, 3H), 1.22 - 1.09 (m, 1H). |
| 1831 | ¹H NMR (400 MHZ, CDC13) 8 8.97 (t, J = 1.8 Hz, 1H), 8.51 (s, |
| | 1H), 8.00 - 7.93 (m, 1H), 7.86 (dt, J = 7.7, 1.4 Hz, 1H), 7.61 (t, |
| | J = 7.8 Hz, 1H), 7.22 (t, J = 7.6 Hz, 1H), 7.06 (d, J = 7.6 Hz, |
| | 2H), 6.58 (s, 1H), 6.20 (s, 1H), 5.55 (dd, J = 11.2, 4.3 Hz, 1H), |
| | 5.42 (d, J = 15.7 Hz, 1H), 4.47 - 4.35 (m, 1H), 4.28 (d, J = 15.8 |
| | Hz, 1H), 4.05 (t, J = 11.4 Hz, 1H), 3.95 (s, 3H), 3.18 (hept, J = |
| | 6.8 Hz, 1H), 2.02 (s, 6H), 1.84 (d, J = 15.0 Hz, 1H), 1.46 (dd, |
| | J = 15.1, 10.2 Hz, 1H), 1.40 (d, J = 6.8 Hz, 3H), 1.38 (d, J = |
| | 6.8 Hz, 3H), 0.54 (s, 3H), 0.39 (dt, J = 9.8, 5.0 Hz, 1H), 0.28 |
| | (dt, J = 9.8, 5.0 Hz, 1H), 0.15 (dt, J = 9.4, 4.9 Hz, 1H), 0.04 |
| | (dt, J = 9.7, 4.9 Hz, 1H). |
| 1832 | ¹H NMR (400 MHZ, CDC13) 8 10.13 (s, 1H), 8.22 (d, J = 7.9 |
| | Hz, 1H), 8.04 (t, J = 7.8 Hz, 1H), 7.89 (d, J = 7.6 Hz, 2H), 7.87 |
| | (s, 1H), 7.21 (t, J = 7.6 Hz, 1H), 7.04 (d, J = 7.7 Hz, 2H), 6.16 |
| | (s, 1H), 5.97 (dd, J = 10.5, 5.0 Hz, 1H), 5.01 (d, J = 15.6 Hz, |
| | 1H), 4.54 (p, J = 8.4 Hz, 1H), 4.33 - 4.18 (m, 2H), 3.98 (t, J = |
| | 11.0 Hz, 1H), 3.13 (s, 3H), 2.33 - 2.11 (m, 4H), 1.98 (s, 6H), |
| | 1.77 - 1.64 (m, 3H), 1.53 (dd, J = 15.1, 1.7 Hz, 1H), 0.60 (s, |
| | 9H). |
| 1833 | ¹H NMR (400 MHZ, CDC13) 8 8.80 (d, J = 2.1 Hz, 1H), 8.01 |
| | (s, 1H), 7.94 (d, J = 7.9 Hz, 1H), 7.79 (d, J = 7.6 Hz, 1H), 7.46 |
| | (s, 1H), 7.18 (t, J = 7.6 Hz, 1H), 7.03 (d, J = 7.6 Hz, 2H), 6.19 |
| | (s, 1H), 5.64 - 5.51 (m, 1H), 4.92 (d, J = 14.8 Hz, 1H), 4.72 (h, |
| | J = 6.3 Hz, 1H), 4.32 - 4.23 (m, 1H), 4.19 (d, J = 6.8 Hz, 2H), |
| | 4.03 (d, J = 14.8 Hz, 1H), 3.96 - 3.85 (m, 1H), 2.50 - 2.41 (m, |
| | 1H), 2.39 (s, 3H), 2.10 - 2.04 (m, 1H), 2.00 (s, 6H), 1.70 (dd, J = |
| | 15.4, 7.1 Hz, 1H), 1.55 (d, J = 15.0 Hz, 1H), 1.48 (d, J = 6.1 |
| | Hz, 3H), 0.62 (s, 9H). |
| 1837 | ¹H NMR (400 MHZ, CDC13) 8 8.77 - 8.73 (m, 1H), 8.05 (d, J = |
| | 7.9 Hz, 1H), 7.92 (s, 1H), 7.87 - 7.80 (m, 2H), 7.66 (t, J = 7.8 |
| | Hz, 1H), 7.21 (t, J = 7.6 Hz, 1H), 7.05 (d, J = 7.6 Hz, 2H), 6.23 |
| | (s, 1H), 5.41 (d, J = 7.7 Hz, 1H), 5.14 (d, J = 15.4 Hz, 1H), |
| | 4.11 - 3.94 (m, 6H), 2.92 (p, J = 10.2 Hz, 2H), 2.52 (ddd, J = |
| | 8.6, 6.4, 2.6 Hz, 2H), 2.16 - 2.06 (m, 2H), 2.03 (s, 7H), 1.91 - |
| | 1.69 (m, 4H), 0.81 (s, 9H). |
| 1838 | ¹H NMR (400 MHZ, CDC13) 8 8.82 (s, 1H), 8.10 (d, J = 7.8 Hz, |
| | 1H), 7.87 (d, J = 7.0 Hz, 2H), 7.73 - 7.67 (m, 2H), 7.22 (d, J = |
| | 7.5 Hz, 1H), 7.09 (d, J = 7.7 Hz, 2H), 6.26 (s, 1H), 5.42 (d, J = |
| | 10.2 Hz, 1H), 5.25 (d, J = 15.7 Hz, 1H), 4.30 (s, 1H), 4.20 (d, |
| | J = 15.7 Hz, 1H), 4.06 - 3.89 (m, 1H), 3.46 (s, 1H), 2.75 (s, |
| | 1H), 2.39 (s, 1H), 2.07 (s, 11H), 1.83 (s, 1H), 1.62 (q, J = 11.1 |
| | Hz, 4H), 1.25 (s, 1H), 0.51 (dd, J = 8.8, 4.7 Hz, 2H), 0.32 - 0.22 |
| | (m, 1H), 0.08 (dd, J = 9.5, 4.8 Hz, 1H), -0.42 (dd, J = 9.6, 4.8 |
| | Hz, 1H). |
| 1839 | ¹H NMR (400 MHZ, CDC13) 8 8.80 - 8.77 (m, 1H), 8.05 (d, J = |
| | 7.8 Hz, 1H), 7.93 (s, 1H), 7.89 - 7.80 (m, 2H), 7.66 (t, J = 7.7 |
| | Hz, 1H), 7.21 (t, J = 7.6 Hz, 1H), 7.07 (d, J = 7.6 Hz, 2H), 6.24 |
| | - 6.21 (m, 1H), 5.41 (dd, J = 11.2, 4.1 Hz, 1H), 5.20 (d, J = |
| | 15.5 Hz, 1H), 4.40 - 4.30 (m, 1H), 4.29 - 4.18 (m, 1H), 4.13 - |
| | 4.02 (m, 2H), 4.00 - 3.92 (m, 1H), 3.41 - 3.30 (m, 1H), 2.77 - |
| | 2.66 (m, 1H), 2.43 - 2.34 (m, 1H), 2.04 - 1.92 (m, 4H), 1.92 - |
| | 1.80 (m, 1H), 1.58 (d, J = 10.4 Hz, 9H), 1.32 - 1.21 (m, 1H), |
| | 0.54 - 0.43 (m, 2H), 0.31 - 0.20 (m, 1H), 0.13 - 0.04 (m, 1H), - |
| | 0.43 (d, J = 4.9 Hz, 1H). |
| 1840 | ¹H NMR (400 MHZ, CDC13) 8 8.85 - 8.82 (m, 1H), 8.03 (d, J = |
| | 7.8 Hz, 1H), 7.91 (s, 1H), 7.85 (d, J = 7.7 Hz, 1H), 7.72 (s, 1H), |
| | 7.66 (t, J = 7.8 Hz, 1H), 7.23 (t, J = 7.6 Hz, 1H), 7.08 (d, J = |
| | 7.7 Hz, 2H), 6.26 (s, 1H), 5.43 (dd, J = 11.1, 4.5 Hz, 1H), 5.19 |
| | (d, J = 16.2 Hz, 1H), 4.57 - 4.47 (m, 1H), 4.40 - 4.29 (m, 1H), |
| | 4.22 (d, J = 16.3 Hz, 1H), 4.00 (t, J = 11.4 Hz, 1H), 3.96 - 3.88 |
| | (m, 1H), 3.50 - 3.39 (m, 1H), 2.78 - 2.69 (m, 1H), 2.41 - 2.33 |
| | (m, 1H), 2.07 - 1.80 (m, 12H), 1.67 - 1.52 (m, 3H), 1.28 - 1.17 |
| | (m, 3H), 0.97 - 0.85 (m, 3H). |
| 1841 | ¹H NMR (400 MHZ, CDC13) 8 8.83 - 8.79 (m, 1H), 8.04 (d, J = |
| | 7.9 Hz, 1H), 7.92 (s, 1H), 7.86 (d, J = 7.7 Hz, 1H), 7.82 (s, 1H), |
| | 7.65 (t, J = 7.7 Hz, 1H), 7.22 (t, J = 7.6 Hz, 1H), 7.08 (d, J = |
| | 7.6 Hz, 2H), 6.27 (s, 1H), 5.46 (dd, J = 11.3, 4.5 Hz, 1H), 5.10 |
| | (d, J = 15.5 Hz, 1H), 4.52 - 4.43 (m, 1H), 4.40 - 4.29 (m, 1H), |
| | 4.14 - 4.03 (m, 2H), 4.00 - 3.91 (m, 1H), 3.41 - 3.29 (m, 1H), |
| | 2.76 - 2.67 (m, 1H), 2.43 - 2.34 (m, 1H), 2.07 - 1.81 (m, 12H), |
| | 1.62 - 1.58 (m, 3H), 1.27 - 1.16 (m, 3H), 0.97 - 0.85 (m, 3H). |
| 1842 | ¹H NMR (400 MHz, CDC13) 8 8.69 (s, 1H), 8.61 (s, 1H), 8.40 |
| | (s, 1H), 8.05 (d, J = 7.8 Hz, 1H), 7.83 (d, J = 7.4 Hz, 1H), 7.66 |
| | (t, J = 7.7 Hz, 1H), 7.21 (t, J = 7.6 Hz, 1H), 7.06 (d, J = 7.6 |
| | Hz, 2H), 6.25 (s, 1H), 5.46 (dd, J = 11.4, 3.8 Hz, 1H), 5.17 (d, |
| | J = 15.0 Hz, 1H), 4.28 - 4.12 (m, 2H), 4.05 (s, 1H), 3.21 - 3.09 |
| | (m, 1H), 2.04 (s, 6H), 1.69 - 1.61 (m, 3H), 1.38 (d, 6H), 1.16 |
| | (g, J = 7.2 Hz, 1H), 0.81 (s, 9H). |
| 1843 | ¹H NMR (400 MHZ, CDC13) 8 8.77 (t, J = 1.8 Hz, 1H), 8.03 (d, |
| | J = 7.8 Hz, 1H), 7.95 (s, 1H), 7.87 - 7.83 (m, 1H), 7.66 (s, 1H), |
| | 7.65 - 7.60 (m, 1H), 7.21 (t, J = 7.6 Hz, 1H), 7.07 (d, J = 7.6 |
| | Hz, 2H), 6.25 (s, 1H), 5.52 - 5.42 (m, 1H), 5.07 (d, J = 15.4 Hz, |
| | 1H), 4.54 - 4.45 (m, 1H), 4.17 - 4.10 (m, 3H), 4.06 (d, J = 15.4 |
| | Hz, 1H), 4.02 - 3.95 (m, 1H), 2.61 - 2.49 (m, 1H), 2.16 - 2.08 |
| | (m, 1H), 2.04 (s, 6H), 1.88 - 1.76 (m, 1H), 1.70 - 1.64 (m, 2H), |
| | 1.56 (d, J = 6.2 Hz, 3H), 1.51 - 1.44 (m, 3H), 1.41 - 1.32 (m, |
| | 2H), 1.22 - 1.13 (m, 1H), 1.08 - 0.99 (m, 1H), 0.46 - 0.33 (m, |
| | 1H). |
| 1844 | ¹H NMR (400 MHZ, CDC13) 8 8.80 (t, J = 1.8 Hz, 1H), 8.05 (d, |
| | J = 7.9 Hz, 1H), 7.89 (s, 2H), 7.88 - 7.84 (m, 1H), 7.65 (t, J = |
| | 7.8 Hz, 1H), 7.21 (t, J = 7.6 Hz, 1H), 7.06 (d, J = 7.6 Hz, 2H), |
| | 6.22 (s, 1H), 5.41 (dd, J = 10.9, 3.8 Hz, 1H), 5.17 (d, J = 15.6 |
| | Hz, 1H), 4.55 (p, J = 8.4 Hz, 1H), 4.21 - 4.12 (m, 1H), 4.09 (d, |
| | J = 15.2 Hz, 1H), 4.03 (d, J = 11.3 Hz, 1H), 3.14 (s, 3H), 2.36 - |
| | 2.26 (m, 2H), 2.25 - 2.14 (m, 2H), 2.03 (s, 6H), 1.88 - 1.80 |
| | (m, 1H), 1.77 - 1.63 (m, 4H), 1.55 - 1.43 (m, 3H), 1.42 - 1.33 |
| | (m, 2H), 1.27 - 1.15 (m, 1H), 1.10 - 0.98 (m, 1H), 0.51 - 0.35 |
| | (m, 1H). |
| 1845 | ¹H NMR (400 MHZ, CDC13) 8 8.76 (t, J = 1.9 Hz, 1H), 8.01 (d, |
| | J = 7.9 Hz, 1H), 7.90 (s, 1H), 7.86 (s, 1H), 7.86 - 7.82 (m, 1H), |
| | 7.63 (t, J = 7.8 Hz, 1H), 7.21 (t, J = 7.6 Hz, 1H), 7.05 (d, J = |
| | 7.6 Hz, 2H), 6.20 (s, 1H), 5.41 (dd, J = 10.5, 3.5 Hz, 1H), 5.16 |
| | (d, J = 15.5 Hz, 1H), 4.18 - 4.09 (m, 1H), 4.09 - 4.01 (m, 2H), |
| | 3.42 - 3.28 (m, 2H), 3.18 (s, 3H), 2.14 - 2.04 (m, 1H), 2.01 (s, |
| | 6H), 1.84 (ddd, J = 14.1, 10.1, 3.7 Hz, 1H), 1.75 - 1.61 (m, 2H), |
| | 1.56 - 1.43 (m, 3H), 1.42 - 1.34 (m, 2H), 1.26 - 1.16 (m, 1H), |
| | 1.10 - 0.99 (m, 1H), 0.92 (d, J = 6.4 Hz, 3H), 0.91 (d, J = 6.5 |
| | Hz, 3H), 0.49 - 0.37 (m, 1H). |
| 1846 | ¹H NMR (400 MHZ, CDC13) 8 8.76 (t, J = 1.9 Hz, 1H), 8.01 (d, |
| | J = 7.9 Hz, 1H), 7.89 (s, 1H), 7.86 (dt, J = 7.7, 1.4 Hz, 1H), |
| | 7.80 (s, 1H), 7.63 (t, J = 7.8 Hz, 1H), 7.21 (t, J = 7.6 Hz, 1H), |
| | 7.06 (d, J = 7.6 Hz, 2H), 6.24 (s, 1H), 5.50 - 5.40 (m, 1H), 5.11 |
| | (d, J = 15.2 Hz, 1H), 4.20 - 4.01 (m, 4H), 3.68 - 3.52 (m, 2H), |
| | 2.40 - 2.28 (m, 1H), 2.03 (s, 6H), 2.01 - 1.98 (m, 1H), 1.98 - |
| | 1.92 (m, 2H), 1.88 - 1.79 (m, 1H), 1.74 - 1.60 (m, 3H), 1.55 - |
| | 1.43 (m, 3H), 1.42 - 1.32 (m, 2H), 1.23 - 1.14 (m, 1H), 1.09 - |
| | 1.00 (m, 1H), 0.91 (d, J = 6.9 Hz, 3H), 0.81 (d, J = 6.8 Hz, 3H), |
| | 0.48 - 0.34 (m, 1H). |
| 1847 | ¹H NMR (400 MHZ, CDC13) 8 8.73 (t, J = 1.8 Hz, 1H), 8.04 - |
| | 7.95 (m, 2H), 7.89 - 7.80 (m, 2H), 7.63 (t, J = 7.8 Hz, 1H), 7.21 |
| | (t, J = 7.6 Hz, 1H), 7.06 (d, J = 7.6 Hz, 2H), 6.21 (s, 1H), 5.36 |
| | (dd, J = 10.8, 3.9 Hz, 1H), 5.13 (d, J = 15.6 Hz, 1H), 4.70 - |
| | 4.58 (m, 1H), 4.24 (d, J = 13.4 Hz, 1H), 4.18 - 4.10 (m, 1H), |
| | 4.08 (d, J = 11.9 Hz, 1H), 4.06 - 3.99 (m, 1H), 3.00 (td, J = |
| | 13.0, 3.1 Hz, 1H), 2.03 (s, 6H), 1.88 - 1.74 (m, 3H), 1.72 - 1.61 |
| | (m, 5H), 1.60 - 1.42 (m, 4H), 1.41 - 1.32 (m, 2H), 1.23 (d, J = |
| | 6.8 Hz, 3H), 1.20 - 1.12 (m, 1H), 1.09 - 0.97 (m, 1H), 0.45 - |
| | 0.32 (m, 1H). |
| 1850 | ¹H NMR (400 MHZ, CDC13) 8 8.77 - 8.73 (m, 1H), 8.07 (d, J = |
| | 8.1 Hz, 1H), 7.89 - 7.84 (m, 3H), 7.67 (t, J = 7.8 Hz, 1H), 7.21 |
| | (t, J = 7.6 Hz, 1H), 7.06 (d, J = 7.6 Hz, 2H), 6.26 (s, 1H), 5.40 |
| | (d, J = 7.1 Hz, 1H), 5.10 (d, J = 15.5 Hz, 1H), 4.81 - 4.71 (m, |
| | 1H), 4.12 - 4.00 (m, 3H), 3.51 - 3.43 (m, 2H), 2.05 (s, 6H), 1.63 - |
| | 1.57 (m, 3H), 1.29 (d, J = 6.7 Hz, 3H), 1.27 - 1.20 (m, 6H), |
| | 1.19 - 1.09 (m, 1H), 0.80 (s, 9H). |
| 1851 | ¹H NMR (400 MHZ, CDC13) 8 8.76 (s, 1H), 8.11 (d, J = 8.0 Hz, |
| | 1H), 7.92 - 7.86 (m, 2H), 7.74 - 7.66 (m, 2H), 7.11 - 7.04 (m, |
| | 3H), 6.32 - 6.26 (m, 1H), 5.51 - 5.43 (m, 1H), 5.16 - 5.07 (m, |
| | 1H), 4.17 - 4.03 (m, 3H), 3.56 - 3.47 (m, 2H), 3.31 - 3.23 (m, |
| | 3H), 3.04 (s, 3H), 2.10 - 2.05 (m, 8H), 1.21 (d, J = 6.8 Hz, 2H), |
| | 1.14 - 1.08 (m, 2H), 0.82 - 0.79 (m, 11H). |
| 1853 | ¹H NMR (400 MHZ, CDC13) 8 9.02 (t, J = 1.7 Hz, 1H), 8.59 (s, |
| | 1H), 8.03 - 7.96 (m, 1H), 7.86 (dt, J = 7.7, 1.4 Hz, 1H), 7.63 (t, |
| | J = 7.8 Hz, 1H), 7.51 (d, J = 3.6 Hz, 1H), 7.22 (t, J = 7.6 Hz, |
| | 1H), 7.07 (d, J = 7.6 Hz, 2H), 6.73 (d, J = 3.6 Hz, 1H), 6.24 (s, |
| | 1H), 5.63 (dd, J = 11.1, 4.2 Hz, 1H), 5.42 (d, J = 15.8 Hz, 1H), |
| | 4.44 (d, J = 15.9 Hz, 1H), 4.27 - 4.18 (m, 1H), 4.08 - 4.00 (m, |
| | 1H), 3.99 (s, 3H), 2.02 (s, 6H), 1.77 (dd, J = 15.1, 8.8 Hz, 1H), |
| | 1.61 (d, J = 14.1 Hz, 1H), 0.67 (s, 9H). |
| 1855 | ¹H NMR (400 MHZ, CDC13) 8 8.86 (s, 1H), 8.07 (d, J = 7.9 Hz, |
| | 1H), 7.92 (s, 1H), 7.87 (d, J = 7.6 Hz, 1H), 7.70 - 7.63 (m, 2H), |
| | 7.24 - 7.18 (m, 1H), 7.08 (d, J = 7.6 Hz, 2H), 6.27 (s, 1H), 5.55 - |
| | 5.48 (m, 1H), 5.12 (d, J = 15.5 Hz, 1H), 4.15 - 3.97 (m, 6H), |
| | 3.79 - 3.70 (m, 2H), 3.31 - 3.22 (m, 2H), 2.62 (s, 2H), 2.45 - |
| | 2.35 (m, 2H), 2.07 (s, 6H), 1.78 - 1.73 (m, 3H), 0.81 (s, 9H). |
| 1856 | ¹H NMR (400 MHZ, CDC13) 8 8.72 (s, 1H), 8.09 - 8.04 (m, 2H), |
| | 7.91 (s, 1H), 7.87 (d, J = 7.6 Hz, 1H), 7.67 (t, J = 7.8 Hz, 1H), |
| | 7.21 (t, J = 7.6 Hz, 1H), 7.07 (d, J = 7.7 Hz, 2H), 6.26 (s, 1H), |
| | 5.35 - 5.27 (m, 1H), 5.14 (d, J = 15.4 Hz, 1H), 4.66 - 4.57 (m, |
| | 1H), 4.40 - 4.29 (m, 2H), 4.13 - 3.95 (m, 4H), 3.64 - 3.51 (m, |
| | 3H), 3.23 (s, 3H), 3.12 - 3.01 (m, 2H), 2.62 (s, 2H), 2.04 (s, |
| | 6H), 1.94 - 1.77 (m, 4H), 0.80 (s, 9H). |
| 1857 | ¹H NMR (400 MHZ, CDC13) 8 8.71 (s, 1H), 8.08 - 8.04 (m, 2H), |
| | 7.91 - 7.83 (m, 2H), 7.67 (t, J = 7.8 Hz, 1H), 7.21 (t, J = 7.6 |
| | Hz, 1H), 7.06 (d, J = 7.6 Hz, 2H), 6.25 (s, 1H), 5.39 (d, J = 7.1 |
| | Hz, 1H), 5.12 (d, J = 15.4 Hz, 1H), 4.68 - 4.61 (m, 1H), 4.36 |
| | (d, J = 13.6 Hz, 1H), 4.09 - 3.97 (m, 3H), 3.59 (d, J = 7.0 Hz, |
| | 2H), 3.29 (s, 3H), 3.07 - 2.98 (m, 1H), 2.62 (s, 2H), 2.04 (s, |
| | 6H), 1.96 - 1.80 (m, 3H), 1.67 (s, 4H), 1.18 - 1.09 (m, 1H), 0.80 |
| | (s, 9H). |
| 1858 | ¹H NMR (400 MHZ, CDC13) 8 8.69 (t, J = 1.8 Hz, 1H), 8.46 (s, |
| | 1H), 8.07 (d, J = 7.9 Hz, 1H), 7.85 (dt, J = 7.8, 1.4 Hz, 1H), |
| | 7.62 (t, J = 7.8 Hz, 1H), 7.19 (t, J = 7.6 Hz, 1H), 7.04 (d, J = |
| | 7.6 Hz, 2H), 6.45 (s, 1H), 6.20 (s, 1H), 5.37 (d, J = 14.7 Hz, |
| | 1H), 5.28 (dd, J = 11.4, 4.3 Hz, 1H), 4.45 - 4.27 (m, 2H), 4.21 - |
| | 4.10 (m, 1H), 3.83 (s, 3H), 3.16 (hept, J = 6.8 Hz, 1H), 1.97 |
| | (s, 6H), 1.89 (dd, J = 15.2, 9.0 Hz, 1H), 1.56 (dd, J = 15.2, 1.6 |
| | Hz, 1H), 1.41 (d, J = 6.7 Hz, 3H), 1.39 (d, J = 6.6 Hz, 3H), 0.64 |
| | (s, 9H). |
| 1859 | ¹H NMR (400 MHZ, CDC13) 8 9.09 (t, J = 1.8 Hz, 1H), 8.48 (d, |
| | J = 8.3 Hz, 1H), 8.03 - 7.95 (m, 1H), 7.84 (dt, J = 7.7, 1.4 Hz, |
| | 1H), 7.65 (t, J = 7.8 Hz, 1H), 7.56 (d, J = 8.4 Hz, 1H), 7.23 (t, |
| | J = 7.6 Hz, 1H), 7.07 (d, J = 7.6 Hz, 2H), 6.21 (s, 1H), 5.66 |
| | (dd, J = 11.1, 4.1 Hz, 1H), 5.45 (d, J = 16.5 Hz, 1H), 4.44 (d, J = |
| | 16.6 Hz, 1H), 4.21 (t, J = 9.9 Hz, 1H), 4.12 (s, 3H), 3.86 (t, |
| | J = 11.3 Hz, 1H), 3.53 (hept, J = 7.0 Hz, 1H), 2.02 (s, 6H), 1.75 - |
| | 1.70 (m, 1H), 1.69 (d, J = 6.9 Hz, 3H), 1.66 (d, J = 6.8 Hz, |
| | 3H), 1.61 (dd, J = 15.1, 1.8 Hz, 1H), 0.69 (s, 9H). |
| 1329 | ¹H NMR (400 MHZ, Chloroform-d) 8 8.86 (d, J = 1.8 Hz, 1H), |
| | 8.05 (d, J = 7.9 Hz, 1H), 7.88 (t, J = 9.4 Hz, 3H), 7.65 (t, J = |
| | 7.8 Hz, 1H), 7.22 (t, J = 7.6 Hz, 1H), 7.08 (d, J = 7.6 Hz, 2H), |
| | 6.27 (s, 1H), 5.46 (dd, J = 11.1, 4.3 Hz, 1H), 5.09 (d, J = 15.5 |
| | Hz, 1H), 4.52 (p, J = 8.4 Hz, 1H), 4.23 (t, J = 9.2 Hz, 1H), 4.14 |
| | (d, J = 15.6 Hz, 1H), 4.06 (t, J = 11.4 Hz, 1H), 3.17 (s, 3H), |
| | 2.35 - 2.26 (m, 2H), 2.26 - 2.17 (m, 2H), 2.03 (s, 6H), 1.78 - |
| | 1.74 (m, 2H), 1.59 - 1.56 (m, 1H), 0.95 (dd, J = 14.1, 7.3 Hz, |
| | 1H), 0.89 - 0.84 (m, 1H), 0.83 - 0.78 (m, 1H), 0.60 (s, 3H), 0.58 - |
| | 0.52 (m, 6H). Sulfonamide N-Hnot visible. |
| 1330 | ¹H NMR (400 MHZ, Chloroform-d) 8 8.86 (t, J = 1.7 Hz, 1H), |
| | 8.10 (d, J = 7.9 Hz, 1H), 7.92 - 7.83 (m, 3H), 7.67 (t, J = 7.8 |
| | Hz, 1H), 7.22 (t, J = 7.6 Hz, 1H), 7.08 (d, J = 7.6 Hz, 2H), 6.29 |
| | (s, 1H), 5.47 (dd, J = 11.1, 4.2 Hz, 1H), 5.08 (d, J = 15.5 Hz, |
| | 1H), 4.54 - 4.46 (m, 1H), 4.25 (s, 1H), 4.16 (d, J = 15.7 Hz, |
| | 1H), 4.06 (t, J = 11.4 Hz, 1H), 3.19 (s, 3H), 2.36 - 2.28 (m, 2H), |
| | 2.25 - 2.18 (m, 2H), 2.03 (s, 6H), 1.77 - 1.73 (m, 2H), 1.61 - |
| | 1.59 (m, 1H), 0.98 - 0.92 (m, 1H), 0.88 - 0.84 (m, 2H), 0.60 (s, |
| | 3H), 0.59 - 0.53 (m, 6H). (Sulfonamide -NH not visible) |
| 1862 | ¹H NMR (400 MHZ, Chloroform-d) 8 8.83 (t, J = 1.9 Hz, 1H), |
| | 8.03 - 7.93 (m, 2H), 7.90 - 7.83 (m, 2H), 7.63 (t, J = 7.8 Hz, |
| | 1H), 7.22 (t, J = 7.6 Hz, 1H), 7.08 (d, J = 7.6 Hz, 2H), 6.27 (s, |
| | 1H), 5.41 (dd, J = 11.2, 4.4 Hz, 1H), 5.04 (d, J = 15.6 Hz, 1H), |
| | 4.73 - 4.64 (m, 1H), 4.24 (t, J = 8.1 Hz, 1H), 4.20 - 4.10 (m, |
| | 2H), 4.04 (t, J = 11.4 Hz, 1H), 3.05 (td, J = 12.9, 3.2 Hz, 1H), |
| | 2.03 (s, 6H), 1.87 - 1.82 (m, 2H), 1.77 - 1.71 (m, 2H), 1.66 - |
| | 1.62 (m, 2H), 1.56 (d, J = 6.2 Hz, 1H), 1.17 (d, J = 6.7 Hz, 3H), |
| | 0.97 - 0.79 (m, 3H), 0.58 (s, 3H), 0.57 - 0.50 (m, 6H). |
| | Sulfonamide NH not visible. |
| 1864 | ¹H NMR (400 MHZ, CDC13) 8 8.74 (s, 1H), 8.02 (d, J = 8.0 Hz, |
| | 1H), 7.90 (s, 1H), 7.81 (d, J = 7.5 Hz, 1H), 7.70 - 7.59 (m, 2H), |
| | 7.20 (t, J = 7.6 Hz, 1H), 7.01 (d, J = 7.6 Hz, 2H), 6.14 (s, 1H), |
| | 5.37 (d, J = 7.6 Hz, 1H), 5.20 (d, J = 15.6 Hz, 1H), 4.36 - 4.26 |
| | (m, 1H), 4.18 - 4.09 (m, 1H), 4.02 - 3.90 (m, 4H), 2.51 - 2.40 |
| | (m, 1H), 2.19 - 2.10 (m, 1H), 2.05 - 1.93 (m, 7H), 1.83 - 1.67 |
| | (m, 3H), 1.50 (d, J = 4.2 Hz, 1H), 1.21 - 1.01 (m, 4H), 0.82 (s, |
| | 9H). |
| 1865 | ¹H NMR (400 MHZ, CDC13) 8 8.67 (s, 1H), 8.02 - 7.90 (m, 2H), |
| | 7.80 (d, J = 7.6 Hz, 1H), 7.68 (s, 1H), 7.62 (t, J = 7.8 Hz, 1H), |
| | 7.20 (t, J = 7.6 Hz, 1H), 7.03 (d, J = 7.6 Hz, 2H), 6.16 (s, 1H), |
| | 5.40 (dd, J = 11.2, 3.9 Hz, 1H), 5.10 (d, J = 15.2 Hz, 1H), 4.38 |
| | - 4.30 (m, 1H), 4.16 - 4.06 (m, 2H), 4.05 - 3.91 (m, 3H), 2.55 - |
| | 2.44 (m, 1H), 2.20 - 2.11 (m, 1H), 2.09 - 2.02 (m, 2H), 2.00 (s, |
| | 6H), 1.89 - 1.68 (m, 3H), 1.63 - 1.58 (m, 2H), 1.19 - 1.09 (m, |
| | 2H), 0.82 (s, 9H). |
| 1331 | ¹H NMR (400 MHZ, Chloroform-d) 8 8.81 (d, J = 1.8 Hz, 1H), |
| | 8.00 (d, J = 7.9 Hz, 1H), 7.86 (s, 1H), 7.82 (dt, J = 7.8, 1.3 Hz, |
| | 1H), 7.76 (s, 1H), 7.62 (t, J = 7.8 Hz, 1H), 7.22 (t, J = 7.6 Hz, |
| | 1H), 7.07 (d, J = 7.6 Hz, 2H), 6.23 (s, 1H), 5.47 (dd, J = 11.1, |
| | 4.1 Hz, 1H), 5.09 (d, J = 15.5 Hz, 1H), 4.29 - 4.17 (m, 2H), |
| | 4.13 - 4.01 (m, 2H), 3.66 (td, J = 8.3, 7.5, 4.3 Hz, 1H), 3.53 - |
| | 3.42 (m, 1H), 2.22 - 2.06 (m, 3H), 2.06 (s, 6H), 1.79 - 1.75 (m, |
| | 1H), 1.67 - 1.65 (m, 1H), 1.64 - 1.62 (m, 1H), 1.61 - 1.59 (m, |
| | 1H), 1.54 - 1.49 (m, 1H), 1.46 - 1.36 (m, 2H), 1.27 (d, J = 6.3 |
| | Hz, 3H), 1.20 (d, J = 12.3 Hz, 1H), 1.14 - 1.07 (m, 1H), 1.04 - |
| | 0.97 (m, 2H), 0.89 (qd, J = 12.1, 3.4 Hz, 2H), 0.40 - 0.27 (m, |
| | 1H). (Sulfonamide -NH not visible) |
| 1866 | ¹H NMR (400 MHZ, Chloroform-d) 8 8.81 (t, J = 1.8 Hz, 1H), |
| | 8.04 (d, J = 7.9 Hz, 1H), 7.89 (d, J = 4.1 Hz, 2H), 7.86 - 7.81 |
| | (m, 1H), 7.64 (t, J = 7.8 Hz, 1H), 7.21 (t, J = 7.6 Hz, 1H), 7.07 |
| | (d, J = 7.6 Hz, 2H), 6.24 (s, 1H), 5.48 (dd, J = 11.1, 4.2 Hz, |
| | 1H), 5.11 (d, J = 15.5 Hz, 1H), 4.28 - 4.19 (m, 1H), 4.08 (dd, J = |
| | 13.4, 9.3 Hz, 2H), 3.98 (t, J = 6.7 Hz, 1H), 3.67 (td, J = 8.9, |
| | 4.5 Hz, 1H), 3.51 (q, J = 9.3, 8.9 Hz, 1H), 2.20 - 2.07 (m, 2H), |
| | 2.04 (s, 6H), 2.02 - 1.96 (m, 1H), 1.95 - 1.89 (m, 1H), 1.68 - |
| | 1.64 (m, 2H), 1.54 - 1.49 (m, 2H), 1.45 - 1.37 (m, 2H), 1.23 - |
| | 1.10 (m, 2H), 1.06 - 0.97 (m, 3H), 0.93 - 0.82 (m, 2H), 0.54 (tq, |
| | J = 9.2, 4.5 Hz, 1H), 0.50 - 0.40 (m, 2H), 0.39 - 0.27 (m, 1H), |
| | 0.20 (dq, J = 10.0, 5.0 Hz, 1H). (Sulfonamide -NH not visible) |
| 1867 | ¹H NMR (400 MHZ, Chloroform-d) 8 8.77 (t, J = 1.9 Hz, 1H), |
| | 8.04 (d, J = 7.9 Hz, 1H), 7.91 (d, J = 6.4 Hz, 2H), 7.83 (d, J = |
| | 7.6 Hz, 1H), 7.64 (t, J = 7.8 Hz, 1H), 7.21 (t, J = 7.6 Hz, 1H), |
| | 7.07 (d, J = 7.6 Hz, 2H), 6.24 (s, 1H), 5.46 (dd, J = 11.1, 4.0 |
| | Hz, 1H), 5.08 (d, J = 15.4 Hz, 1H), 4.27 - 4.19 (m, 2H), 4.09 |
| | (dd, J = 13.1, 10.3 Hz, 2H), 3.71 - 3.64 (m, 1H), 3.55 (dd, J = |
| | 9.2, 4.4 Hz, 1H), 3.49 (d, J = 8.8 Hz, 1H), 3.39 - 3.34 (m, 1H), |
| | 3.33 (s, 3H), 2.14 - 2.06 (m, 4H), 2.04 (s, 6H), 1.62 (t, J = 3.2 |
| | Hz, 2H), 1.49 (d, J = 13.4 Hz, 2H), 1.43 - 1.36 (m, 2H), 1.20 - |
| | 1.10 (m, 2H), 1.01 (t, J = 10.1 Hz, 2H), 0.92 - 0.83 (m, 2H), |
| | 0.33 (q, J = 11.4 Hz, 1H). (Sulfonamide -NH not visible) |
| 1868 | ¹H NMR (400 MHZ, CDC13) 8 8.74 (s, 1H), 8.09 - 8.03 (m, 2H), |
| | 7.98 (s, 1H), 7.85 (d, J = 7.6 Hz, 1H), 7.66 (t, J = 7.8 Hz, 1H), |
| | 7.21 (t, J = 7.6 Hz, 1H), 7.07 (d, J = 7.6 Hz, 2H), 6.26 (s, 1H), |
| | 5.44 (dd, J = 11.3, 4.0 Hz, 1H), 5.10 (d, J = 15.4 Hz, 1H), 4.52 - |
| | 4.44 (m, 2H), 4.19 - 3.95 (m, 7H), 3.73 (d, J = 5.3 Hz, 2H), |
| | 3.61 (p, J = 6.0 Hz, 2H), 2.05 (s, 6H), 1.19 - 1.13 (m, 8H), 0.80 |
| | (s, 9H). |
| 1869 | ¹H NMR (400 MHZ, CDC13) 8 8.78 (t, J = 1.8 Hz, 1H), 7.92 (d, |
| | J = 8.2 Hz, 1H), 7.89 (s, 1H), 7.87 (s, 1H), 7.84 (dt, J = 7.7, 1.4 |
| | Hz, 1H), 7.58 (t, J = 7.8 Hz, 1H), 7.21 (t, J = 7.6 Hz, 1H), 7.06 |
| | (d, J = 7.6 Hz, 2H), 6.19 (s, 1H), 5.42 (dd, J = 11.2, 4.4 Hz, |
| | 1H), 5.05 (d, J = 15.7 Hz, 1H), 4.59 - 4.46 (m, 1H), 4.45 - 4.34 |
| | (m, 1H), 4.18 (d, J = 15.7 Hz, 1H), 4.06 (t, J = 11.5 Hz, 1H), |
| | 3.13 (s, 3H), 2.34 - 2.24 (m, 2H), 2.24 - 2.12 (m, 2H), 2.01 (s, |
| | 6H), 1.91 (dd, J = 15.2, 9.0 Hz, 1H), 1.77 - 1.72 (m, 2H), 1.70 |
| | (d, J = 14.7 Hz, 1H), 1.06 (s, 3H), 0.72 (s, 3H). |
| 1332 | ¹H NMR (400 MHZ, DMSO-d6) § 13.02 (br. s., 1H), 8.68 (s, |
| | 1H), 8.01 - 7.92 (m, 1H), 7.87 (s, 1H), 7.80 - 7.63 (m, 3H), 7.31 - |
| | 7.22 (m, 1H), 7.19 - 7.07 (m, 2H), 6.44 (br. s., 1H), 5.45 - 5.30 |
| | (m, 1H), 4.91 - 4.68 (m, 2H), 4.46 (dd, J = 15.8, 4.3 Hz, 1H), |
| | 4.35 - 4.21 (m, 1H), 4.10 - 3.99 (m, 1H), 3.96 - 3.87 (m, 2H), |
| | 3.86 - 3.74 (m, 1H), 3.67 - 3.44 (m, 2H), 2.38 (q, J = 8.6 Hz, |
| | 1H), 2.26 - 1.84 (m, 8H), 1.83 - 1.68 (m, 2H), 1.61 (quin, J = |
| | 9.5 Hz, 1H), 1.41 (dd, J = 14.9, 5.4 Hz, 1H), 1.30 - 1.16 (m, |
| | 1H), 0.56 (s, 9H). |
| 1870 | ¹H NMR (400 MHz, DMSO-d6) § 12.87 (br. s., 1H), 11.85 (d, |
| | J = 1.5 Hz, 1H), 8.53 (s, 1H), 8.43 (s, 1H), 8.01 - 7.89 (m, 1H), |
| | 7.75 - 7.63 (m, 2H), 7.31 - 7.22 (m, 1H), 7.18 - 7.08 (m, 2H), |
| | 6.52 - 6.41 (m, 1H), 6.33 (d, J = 1.2 Hz, 1H), 5.31 (dd, J = 10.8, |
| | 4.2 Hz, 1H), 4.93 (d, J = 15.2 Hz, 1H), 4.60 (d, J = 14.9 Hz, |
| | 1H), 4.42 (t, J = 11.0 Hz, 1H), 4.16 - 4.03 (m, 1H), 3.11 (spt, J = |
| | 6.8 Hz, 1H), 2.21 - 1.81 (m, 7H), 1.48 - 1.38 (m, 1H), 1.33 |
| | (d, J = 6.8 Hz, 6H), 0.56 (s, 9H). |
| 1871 | ¹H NMR (400 MHz, DMSO-d6) 8 13.11 (br. s, 1H), 8.58 (br. s, |
| | 1H), 7.97 (br. s, 1H), 7.72 (br. s, 2H), 7.31 - 7.23 (m, 1H), 7.21 - |
| | 7.06 (m, 3H), 6.79 (s, 1H), 6.76 - 6.63 (m, 2H), 6.40 (br. s, |
| | 1H), 5.05 (dd, J = 9.8, 3.4 Hz, 1H), 4.90 (d, J = 15.7 Hz, 1H), |
| | 4.41 (d, J = 15.7 Hz, 1H), 4.18 - 3.96 (m, 3H), 2.80 (s, 3H), |
| | 2.28 - 1.76 (m, 11H), 1.71 - 1.57 (m, 2H), 1.36 (d, J = 15.2 Hz, |
| | 1H), 0.53 (s, 9H). |
| 1333 | ¹H NMR (400 MHZ, CDC13) 8 8.75 (t, J = 1.8 Hz, 1H), 8.40 (s, |
| | 1H), 8.03 (dt, J = 8.0, 1.4 Hz, 1H), 7.82 (dt, J = 7.7, 1.4 Hz, |
| | 1H), 7.60 (t, J = 7.8 Hz, 1H), 7.21 (t, J = 7.6 Hz, 1H), 7.04 (d, |
| | J = 7.6 Hz, 2H), 6.63 (d, J = 1.0 Hz, 1H), 6.21 (s, 1H), 5.45 |
| | (dd, J = 10.9, 3.9 Hz, 1H), 5.26 (d, J = 14.8 Hz, 1H), 4.33 (d, J = |
| | 14.9 Hz, 1H), 4.26 (t, J = 11.3 Hz, 1H), 4.21 - 4.09 (m, 1H), |
| | 3.23 - 3.09 (m, 1H), 1.97 (s, 6H), 1.80 (dd, J = 15.2, 8.6 Hz, |
| | 1H), 1.58 (dd, J = 15.1, 1.6 Hz, 1H), 1.40 (d, J = 6.9 Hz, 6H), |
| | 0.64 (s, 9H). |
| 1334 | ¹H NMR (400 MHZ, CDC13) 8 8.94 (t, J = 1.8 Hz, 1H), 8.48 (s, |
| | 1H), 7.99 (d, J = 7.9 Hz, 1H), 7.89 - 7.81 (m, 1H), 7.63 (t, J = |
| | 7.8 Hz, 1H), 7.21 (t, J = 7.6 Hz, 1H), 7.04 (d, J = 7.6 Hz, 2H), |
| | 6.48 (s, 1H), 6.18 (s, 1H), 5.56 - 5.50 (m, 1H), 5.48 (d, J = 15.9 |
| | Hz, 1H), 4.35 (d, J = 15.8 Hz, 1H), 4.16 - 4.06 (m, 1H), 4.01 (t, |
| | J= 11.2 Hz, 1H), 3.91 (s, 3H), 3.16 (hept, J = 6.8 Hz, 1H), 2.01 |
| | (s, 6H), 1.91 (ddd, J = 14.4, 10.5, 3.8 Hz, 1H), 1.79 - 1.68 (m, |
| | 2H), 1.63 - 1.47 (m, 3H), 1.47 - 1.41 (m, 2H), 1.40 (d, J = 6.7 |
| | Hz, 3H), 1.37 (d, J = 6.8 Hz, 3H), 1.33 - 1.25 (m, 1H), 1.14 - |
| | 1.03 (m, 1H), 0.58 - 0.46 (m, 1H). |
| 1872 | ¹H NMR (400 MHZ, CDC13) 8 8.39 (s, 1H), 7.91 (d, J = 8.0 Hz, |
| | 2H), 7.87 (d, J = 7.8 Hz, 1H), 7.49 (d, J = 7.5 Hz, 1H), 7.40 (t, |
| | J = 7.7 Hz, 1H), 7.12 (t, J = 7.6 Hz, 1H), 6.97 (d, J = 7.6 Hz, |
| | 2H), 5.91 (s, 1H), 5.00 (d, J = 15.0 Hz, 1H), 4.72 (p, J = 8.5 |
| | Hz, 1H), 4.53 (d, J = 15.1 Hz, 1H), 4.44 (d, J = 10.1 Hz, 1H), |
| | 3.66 - 3.51 (m, 2H), 3.36 (dd, J = 14.9, 5.2 Hz, 1H), 3.06 (s, |
| | 3H), 2.30 - 2.17 (m, 4H), 1.99 (s, 7H), 1.79 - 1.68 (m, 2H), 1.47 |
| | (dd, J = 14.4, 4.3 Hz, 1H), 0.94 (dd, J = 14.4, 5.3 Hz, 1H), 0.75 |
| | (s, 9H). |
| 1873 | ¹H NMR (400 MHZ, CDC13) 8 8.41 (s, 1H), 7.91 (s, 2H), 7.87 |
| | (d, J = 7.6 Hz, 1H), 7.47 (d, J = 7.5 Hz, 1H), 7.41 (t, J = 7.6 |
| | Hz, 1H), 7.12 (t, J = 7.6 Hz, 1H), 6.99 (d, J = 7.6 Hz, 2H), 5.86 |
| | (s, 1H), 5.02 (d, J = 15.0 Hz, 1H), 4.80 - 4.66 (m, 1H), 4.51 (d, |
| | J = 15.1 Hz, 1H), 4.43 (dd, J = 11.2, 4.1 Hz, 1H), 3.67 - 3.53 |
| | (m, 2H), 3.34 (dd, J = 15.0, 5.3 Hz, 1H), 3.06 (s, 3H), 2.29 - |
| | 2.15 (m, 4H), 2.00 (broad, 7H), 1.79 - 1.70 (m, 2H), 1.49 (dd, J |
| | = 14.4, 4.4 Hz, 1H), 0.94 (dd, J = 14.4, 5.2 Hz, 1H), 0.75 (s, |
| | 9H). |
| 1874 | ¹H NMR (400 MHZ, CDC13) 8 8.50 (s, 1H), 7.99 (d, J = 7.9 Hz, |
| | 1H), 7.93 (s, 1H), 7.87 (s, 1H), 7.82 (d, J = 7.7 Hz, 1H), 7.53 |
| | (t, J = 7.7 Hz, 1H), 7.18 (t, J = 7.6 Hz, 1H), 7.04 (d, J = 7.6 |
| | Hz, 2H), 6.16 (s, 1H), 6.05 (t, J = 11.7 Hz, 1H), 4.88 (d, J = |
| | 15.5 Hz, 1H), 4.69 (p, J = 8.5 Hz, 1H), 4.38 - 4.29 (m, 1H), |
| | 4.24 - 4.18 (m, 1H), 4.16 (d, J = 15.4 Hz, 1H), 3.04 (s, 3H), |
| | 2.90 (d, J = 14.7 Hz, 1H), 2.26 - 2.15 (m, 5H), 1.99 (s, 6H), |
| | 1.80 - 1.71 (m, 3H), 1.34 (dd, J = 15.0, 3.2 Hz, 1H), 0.54 (s, |
| | 9H). |
| 1875 | ¹H NMR (400 MHZ, CDC13) 8 8.43 (s, 1H), 7.99 (d, J = 7.9 Hz, |
| | 1H), 7.95 (s, 1H), 7.84 (s, 1H), 7.61 (d, J = 7.6 Hz, 1H), 7.20 |
| | (s, 1H), 7.07 (t, J = 7.6 Hz, 1H), 6.92 (d, J = 7.6 Hz, 2H), 6.04 |
| | (s, 1H), 5.96 (s, 1H), 4.84 (d, J = 15.5 Hz, 1H), 4.69 (p, J = 8.6 |
| | Hz, 1H), 4.37 - 4.25 (m, 1H), 4.18 (d, J = 15.5 Hz, 1H), 4.05 |
| | (d, J = 10.7 Hz, 1H), 3.03 (s, 3H), 2.75 (d, J = 14.5 Hz, 1H), |
| | 2.20 - 2.12 (m, 5H), 1.89 (s, 6H), 1.77 - 1.67 (m, 3H), 1.41 - |
| | 1.32 (m, 1H), 0.55 (s, 9H). |
| 1876 | ¹H NMR (400 MHZ, CDC13) 8 8.77 (s, 1H), 8.13 (s, 1H), 8.08 |
| | (d, J = 7.9 Hz, 1H), 7.92 (s, 1H), 7.87 (d, J = 7.6 Hz, 1H), 7.68 |
| | (t, J = 7.7 Hz, 1H), 7.21 (t, J = 7.6 Hz, 1H), 7.07 (d, J = 7.6 |
| | Hz, 2H), 6.26 (s, 1H), 5.43 (d, J = 8.3 Hz, 1H), 5.14 (d, J = |
| | 15.2 Hz, 1H), 4.17 - 3.97 (m, 4H), 3.66 - 3.53 (m, 3H), 3.24 - |
| | 3.19 (m, 5H), 3.18 - 3.05 (m, 3H), 2.05 (s, 6H), 1.21 - 1.10 (m, |
| | 2H), 0.81 (s, 9H), 0.60 - 0.50 (m, 4H). |
| 1877 | ¹H NMR (400 MHZ, DMSO-d6) 8 12.87 (br. s, 1H), 8.81 (s, |
| | 1H), 8.43 (s, 1H), 7.94 (d, J = 6.1 Hz, 1H), 7.73 - 7.62 (m, 2H), |
| | 7.29 - 7.21 (m, 1H), 7.17 - 7.09 (m, 2H), 6.42 (d, J = 1.0 Hz, |
| | 2H), 5.59 (dd, J = 10.3, 3.9 Hz, 1H), 5.04 (d, J = 15.9 Hz, 1H), |
| | 4.70 (d, J = 15.9 Hz, 1H), 4.30 (t, J = 11.4 Hz, 1H), 4.09 - 3.96 |
| | (m, 1H), 3.81 (s, 3H), 2.51 (br. s, 3H, overlapped with DMSO), |
| | 2.25 - 1.88 (m, 6H), 1.83 (dd, J = 15.4, 8.8 Hz, 1H), 1.48 (d, J |
| | = 14.9 Hz, 1H), 0.61 (s, 9H). |
| 1878 | ¹H NMR (400 MHZ, DMSO-d6) 8 12.99 (br. s, 1H), 8.82 (s, |
| | 1H), 8.44 (s, 1H), 7.95 (d, J = 6.4 Hz, 1H), 7.76 - 7.61 (m, 2H), |
| | 7.31 - 7.22 (m, 1H), 7.18 - 7.08 (m, 2H), 6.43 (s, 2H), 5.56 (dd, |
| | J = 10.9, 3.8 Hz, 1H), 5.03 (d, J = 16.1 Hz, 1H), 4.72 (d, J = |
| | 16.4 Hz, 1H), 4.49 - 4.27 (m, 3H), 4.12 - 4.03 (m, 1H), 3.23 |
| | (spt, J = 6.8 Hz, 1H), 2.28 - 1.87 (m, 6H), 1.82 (dd, J = 14.9, |
| | 9.0 Hz, 1H), 1.47 (d, J = 14.7 Hz, 1H), 1.37 - 1.27 (m, 9H), |
| | 0.60 (s, 9H). |
| 1879 | ¹H NMR (400 MHZ, CDC13) 8 8.67 - 8.62 (m, 2H), 8.41 (s, 1H), |
| | 8.12 (d, J = 8.0 Hz, 1H), 7.81 - 7.75 (m, 1H), 7.65 (t, J = 7.8 |
| | Hz, 1H), 7.19 (t, J = 7.6 Hz, 1H), 7.00 (d, J = 7.6 Hz, 2H), 6.17 |
| | - 6.13 (m, 1H), 5.45 - 5.37 (m, 1H), 5.18 (d, J = 15.1 Hz, 1H), |
| | 4.22 - 4.08 (m, 4H), 4.07 - 3.98 (m, 1H), 3.64 - 3.54 (m, 2H), |
| | 3.10 - 3.00 (m, 1H), 2.12 - 1.83 (m, 11H), 1.68 - 1.58 (m, 2H), |
| | 1.19 - 1.09 (m, 1H), 0.83 (s, 9H). |
| 1881 | ¹H NMR (400 MHZ, CDC13) 8 8.74 - 8.66 (m, 3H), 8.09 (d, J = |
| | 7.8 Hz, 1H), 7.86 (d, J = 7.7 Hz, 1H), 7.68 (t, J = 7.8 Hz, 1H), |
| | 7.24 - 7.17 (m, 1H), 7.07 (d, J = 7.6 Hz, 2H), 6.28 (s, 1H), 5.35 |
| | (dd, J = 10.9, 3.6 Hz, 1H), 5.23 (d, J = 15.3 Hz, 1H), 4.53 (d, J |
| | = 11.2 Hz, 1H), 4.22 - 4.02 (m, 5H), 3.71 - 3.60 (m, 2H), 2.62 |
| | (s, 4H), 2.20 - 2.12 (m, 1H), 2.06 (s, 6H), 1.77 - 1.68 (m, 3H), |
| | 0.80 (s, 9H). |
| 1335 | ¹H NMR (400 MHZ, CDC13) 8 8.77 (s, 1H), 8.04 (d, J = 7.9 Hz, |
| | 1H), 7.90 (d, J = 7.4 Hz, 2H), 7.84 (d, J = 7.5 Hz, 1H), 7.65 (t, |
| | J = 7.8 Hz, 1H), 7.22 (t, J = 7.6 Hz, 1H), 7.06 (d, J = 7.6 Hz, |
| | 2H), 6.24 (s, 1H), 5.40 (d, J = 7.1 Hz, 1H), 5.14 (d, J = 15.0 |
| | Hz, 1H), 4.61 - 4.47 (m, 1H), 4.15 - 3.99 (m, 3H), 3.15 (s, 3H), |
| | 2.34 - 2.27 (m, 2H), 2.26 - 2.17 (m, 2H), 2.04 (s, 6H), 1.76 - |
| | 1.71 (m, 4H), 1.21 - 1.12 (m, 1H), 0.91 (s, 3H), 0.87 - 0.81 (m, |
| | 1H), 0.21 - 0.12 (m, 2H), 0.12 - 0.05 (m, 2H). |
| 1336 | ¹H NMR (400 MHZ, CDC13) 8 8.76 (s, 1H), 8.03 (d, J = 7.9 Hz, |
| | 1H), 7.90 (d, J = 7.4 Hz, 2H), 7.84 (d, J = 7.6 Hz, 1H), 7.65 (t, |
| | J = 7.8 Hz, 1H), 7.22 (t, J = 7.6 Hz, 1H), 7.06 (d, J = 7.6 Hz, |
| | 2H), 6.24 (s, 1H), 5.40 (d, J = 7.0 Hz, 1H), 5.15 (d, J = 14.8 |
| | Hz, 1H), 4.62 - 4.46 (m, 1H), 4.17 - 3.97 (m, 3H), 3.15 (s, 3H), |
| | 2.35 - 2.27 (m, 2H), 2.26 - 2.17 (m, 2H), 2.04 (s, 6H), 1.77 - |
| | 1.71 (m, 4H), 1.22 - 1.13 (m, 1H), 0.91 (s, 3H), 0.88 - 0.82 (m, |
| | 1H), 0.21 - 0.12 (m, 2H), 0.12 - 0.05 (m, 2H). |
| 1882 | ¹H NMR (400 MHZ, CDC13) 8 8.74 (s, 1H), 8.70 (t, J = 1.8 Hz, |
| | 1H), 8.11 (d, J = 7.9 Hz, 1H), 7.84 (d, J = 7.6 Hz, 1H), 7.63 (t, |
| | J = 7.8 Hz, 1H), 7.21 (t, J = 7.6 Hz, 1H), 7.05 (d, J = 7.6 Hz, |
| | 2H), 6.63 (s, 1H), 6.24 (s, 1H), 5.45 (dd, J = 11.4, 4.3 Hz, 1H), |
| | 5.20 (d, J = 14.6 Hz, 1H), 4.41 (t, J = 11.6 Hz, 1H), 4.28 (d, J = |
| | 14.6 Hz, 1H), 4.25 - 4.16 (m, 1H), 3.16 (hept, J = 6.9 Hz, |
| | 1H), 1.99 (s, 6H), 1.78 (dd, J = 15.2, 8.8 Hz, 1H), 1.59 (d, J = |
| | 15.1 Hz, 1H), 1.41 (d, J = 6.9 Hz, 6H), 0.63 (s, 9H). |
| 1883 | ¹H NMR (400 MHZ, CDC13) 8 8.89 (t, J = 1.8 Hz, 1H), 7.94 (d, |
| | J = 7.9 Hz, 1H), 7.84 (dt, J = 7.7, 1.4 Hz, 1H), 7.61 (t, J = 7.8 |
| | Hz, 1H), 7.59 (s, 1H), 7.22 (t, J = 7.6 Hz, 1H), 7.07 (d, J = 7.6 |
| | Hz, 2H), 6.23 (s, 1H), 5.53 (d, J = 7.3 Hz, 1H), 5.02 (d, J = |
| | 15.5 Hz, 1H), 4.21 - 4.01 (m, 3H), 2.99 (s, 5H), 2.01 (s, 6H), |
| | 1.70 (dd, J = 15.2, 7.8 Hz, 1H), 1.55 (d, J = 15.0 Hz, 1H), 1.35 |
| | (s, 3H), 1.34 (s, 3H), 0.62 (s, 9H). |
| 1886 | ¹H NMR (400 MHZ, Chloroform-d) & 8.80 (t, J = 1.8 Hz, 1H), |
| | 8.04 (d, J = 7.9 Hz, 1H), 7.92 (s, 1H), 7.82 (dd, J = 7.8, 1.4 Hz, |
| | 1H), 7.70 (s, 1H), 7.63 (t, J = 7.7 Hz, 1H), 7.21 (t, J = 7.6 Hz, |
| | 1H), 7.06 (d, J = 7.6 Hz, 2H), 6.21 (s, 1H), 5.40 (dd, J = 11.3, |
| | 4.1 Hz, 1H), 5.14 (d, J = 15.6 Hz, 1H), 4.29 - 4.08 (m, 3H), |
| | 4.08 - 3.92 (m, 3H), 2.39 - 2.27 (m, 1H), 2.22 (dq, J = 10.9, 5.6, |
| | 4.8 Hz, 2H), 2.03 (s, 6H), 1.69 - 1.49 (m, 6H), 1.46 - 1.35 (m, |
| | 2H), 1.23 - 1.09 (m, 2H), 1.05 - 0.98 (m, 4H), 0.92 - 0.86 (m, |
| | 4H), 0.34 (q, J = 10.7, 9.7 Hz, 1H). Sulfonamide NH not visible |
| 1887 | ¹H NMR (400 MHZ, Chloroform-d) & 8.73 (t, J = 1.8 Hz, 1H), |
| | 8.03 (d, J = 7.9 Hz, 1H), 7.95 (s, 1H), 7.82 (d, J = 7.6 Hz, 1H), |
| | 7.70 (s, 1H), 7.63 (t, J = 7.8 Hz, 1H), 7.21 (t, J = 7.6 Hz, 1H), |
| | 7.07 (d, J = 7.6 Hz, 2H), 6.23 (s, 1H), 5.42 (dd, J = 10.3, 3.2 |
| | Hz, 1H), 5.06 (d, J = 15.3 Hz, 1H), 4.31 (dt, J = 8.5, 5.8 Hz, |
| | 1H), 4.16 (dd, J = 14.9, 10.5 Hz, 2H), 4.13 - 3.97 (m, 3H), 2.41 - |
| | 2.18 (m, 3H), 2.03 (s, 6H), 1.66 - 1.56 (m, 4H), 1.52 - 1.35 |
| | (m, 4H), 1.22 - 1.07 (m, 2H), 1.04 - 0.98 (m, 4H), 0.93 - 0.86 |
| | (m, 4H), 0.33 (q, J = 10.8, 10.3 Hz, 1H). Sulfonamide NH not |
| | visible |
| 1888 | ¹H NMR (400 MHZ, CDC13) 8 8.81 (t, J = 1.9 Hz, 1H), 7.95 (d, |
| | J = 7.9 Hz, 1H), 7.87 - 7.81 (m, 2H), 7.76 (s, 1H), 7.61 (t, J = |
| | 7.7 Hz, 1H), 7.22 (t, J = 7.6 Hz, 1H), 7.06 (d, J = 7.6 Hz, 2H), |
| | 6.21 (s, 1H), 5.48 (dd, J = 10.2, 3.0 Hz, 1H), 5.12 (d, J = 15.6 |
| | Hz, 1H), 4.21 (t, J = 6.4 Hz, 1H), 4.17 - 4.02 (m, 3H), 3.70 - |
| | 3.59 (m, 1H), 3.53 - 3.43 (m, 1H), 2.13 - 2.05 (m, 3H), 2.03 (s, |
| | 6H), 1.87 - 1.76 (m, 2H), 1.73 - 1.60 (m, 2H), 1.58 - 1.42 (m, |
| | 3H), 1.42 - 1.31 (m, 2H), 1.26 (d, J = 6.3 Hz, 3H), 1.23 - 1.14 |
| | (m, 1H), 1.10 - 0.98 (m, 1H), 0.42 (dq, J = 12.3, 7.9 Hz, 1H). |
| 1890 | ¹H NMR (400 MHZ, CDC13) 8 8.74 (s, 1H), 7.86 (s, 1H), 7.86 - |
| | 7.82 (m, 1H), 7.81 - 7.78 (m, 1H), 7.75 (s, 1H), 7.59 (t, J = 7.7 |
| | Hz, 1H), 7.24 - 7.20 (m, 1H), 7.09 (d, J = 7.6 Hz, 2H), 6.26 - |
| | 6.24 (m, 1H), 5.42 - 5.38 (m, 1H), 5.16 - 5.10 (m, 1H), 4.37 - |
| | 4.21 (m, 4H), 3.96 - 3.89 (m, 1H), 3.74 - 3.59 (m, 4H), 3.53 - |
| | 3.45 (m, 1H), 3.21 - 3.16 (m, 1H), 2.08 (s, 6H), 2.05 - 2.00 (m, |
| | 4H), 1.91 - 1.80 (m, 2H), 0.53 - 0.40 (m, 5H). |
| 1891 | ¹H NMR (400 MHZ, CDC13) 8 8.68 (s, 1H), 7.85 (s, 1H), 7.81 - |
| | 7.79 (m, 1H), 7.78 - 7.74 (m, 1H), 7.46 (t, J = 7.8 Hz, 1H), 7.39 - |
| | 7.34 (m, 1H), 7.25 - 7.21 (m, 1H), 7.09 - 7.04 (m, 2H), 6.19 |
| | (s, 1H), 5.43 - 5.37 (m, 1H), 5.07 (d, J = 15.4 Hz, 1H), 4.34 - |
| | 4.19 (m, 4H), 3.95 - 3.90 (m, 1H), 3.70 - 3.56 (m, 4H), 3.48 - |
| | 3.40 (m, 1H), 3.18 - 3.12 (m, 1H), 1.91 - 1.83 (m, 2H), 1.02 - |
| | 0.91 (m, 2H), 0.55 - 0.29 (m, 11H), 0.19 - 0.02 (m, 2H). |
| 1895 | ¹H NMR (400 MHZ, CDC13) 8 8.74 (s, 1H), 8.15 (d, J = 1.6 Hz, |
| | 1H), 7.92 - 7.84 (m, 3H), 7.69 (t, J = 7.8 Hz, 1H), 7.19 (t, J = |
| | 7.6 Hz, 1H), 7.03 (d, J = 7.6 Hz, 2H), 6.07 (s, 1H), 5.47 - 5.38 |
| | (m, 1H), 5.12 (d, J = 15.6 Hz, 1H), 4.62 - 4.50 (m, 1H), 4.13 (t, |
| | J = 11.4 Hz, 1H), 4.00 - 3.90 (m, 2H), 3.12 (s, 3H), 2.76 - 2.64 |
| | (m, 1H), 2.35 - 2.11 (m, 5H), 1.99 (s, 6H), 1.65 - 1.56 (m, 1H), |
| | 1.56 - 1.47 (m, 1H), 1.10 (s, 3H), 0.90 - 0.80 (m, 3H), 0.78 (s, |
| | 3H). |
| 1896 | ¹H NMR (400 MHz, CDC13) 8 8.75 (s, 1H), 8.14 (d, J = 7.9 Hz, |
| | 1H), 7.93 - 7.84 (m, 3H), 7.70 (t, J = 7.8 Hz, 1H), 7.20 (t, J = |
| | 7.6 Hz, 1H), 7.05 (d, J = 7.6 Hz, 2H), 6.10 (s, 1H), 5.44 (dd, J = |
| | 11.3, 4.0 Hz, 1H), 5.11 (d, J = 15.5 Hz, 1H), 4.64 - 4.50 (m, |
| | 1H), 4.15 (t, J = 11.4 Hz, 1H), 4.02 - 3.91 (m, 2H), 3.13 (s, 3H), |
| | 2.76 - 2.65 (m, 1H), 2.34 - 2.14 (m, 4H), 2.01 (s, 8H), 1.54 - |
| | 1.47 (m, 1H), 1.10 (s, 3H), 0.92 - 0.81 (m, 3H), 0.77 (s, 3H). |
| 1337 | ¹H NMR (400 MHZ, CDC13) 8 8.81 (s, 1H), 8.13 - 8.09 (m, 1H), |
| | 7.91 - 7.86 (m, 1H), 7.83 (s, 1H), 7.74 (s, 1H), 7.68 (t, J = 7.8 |
| | Hz, 1H), 7.20 (t, J = 7.6 Hz, 1H), 7.05 (d, J = 7.6 Hz, 2H), 6.11 |
| | (s, 1H), 5.51 (dd, J = 11.3, 3.9 Hz, 1H), 5.09 (d, J = 15.8 Hz, |
| | 1H), 4.28 - 4.20 (m, 1H), 4.12 (t, J = 11.4 Hz, 1H), 4.02 - 3.94 |
| | (m, 2H), 3.70 - 3.63 (m, 1H), 3.50 - 3.39 (m, 1H), 2.74 - 2.60 |
| | (m, 1H), 2.18 - 2.03 (m, 4H), 2.01 (s, 6H), 1.87 - 1.71 (m, 6H), |
| | 1.11 (s, 3H), 0.89 - 0.81 (m, 1H), 0.77 (s, 3H). |
| 1338 | ¹H NMR (400 MHZ, CDC13) 8 8.76 (s, 1H), 8.11 - 8.06 (m, 1H), |
| | 7.90 - 7.85 (m, 1H), 7.83 (s, 1H), 7.76 (s, 1H), 7.67 (t, J = 7.8 |
| | Hz, 1H), 7.20 (t, J = 7.6 Hz, 1H), 7.05 (d, J = 7.6 Hz, 2H), 6.09 |
| | (s, 1H), 5.50 (dd, J = 11.4, 4.0 Hz, 1H), 5.06 (d, J = 15.5 Hz, |
| | 1H), 4.24 - 4.14 (m, 2H), 4.01 - 3.91 (m, 2H), 3.67 - 3.60 (m, |
| | 1H), 3.51 - 3.42 (m, 2H), 2.76 - 2.63 (m, 1H), 2.13 - 2.05 (m, |
| | 4H), 2.01 (s, 6H), 1.27 (d, J = 6.3 Hz, 3H), 1.19 (t, J = 10.2 Hz, |
| | 2H), 1.11 (s, 3H), 0.89 - 0.82 (m, 1H), 0.77 (s, 3H). |
| 1897 | ¹H NMR (400 MHZ, CDC13) 8 8.73 (s, 1H), 8.12 - 8.07 (m, 1H), |
| | 7.98 (s, 1H), 7.90 - 7.83 (m, 2H), 7.68 (t, J = 7.8 Hz, 1H), 7.20 |
| | (t, J = 7.6 Hz, 1H), 7.06 (d, J = 7.6 Hz, 2H), 6.11 (s, 1H), 5.43 |
| | (dd, J = 11.2, 4.0 Hz, 1H), 5.04 (d, J = 15.6 Hz, 1H), 4.73 - |
| | 4.65 (m, 1H), 4.21 - 4.09 (m, 2H), 4.02 - 3.92 (m, 2H), 3.03 (td, |
| | J = 12.9, 3.2 Hz, 1H), 2.75 - 2.61 (m, 1H), 2.02 (s, 6H), 1.85 - |
| | 1.69 (m, 8H), 1.16 (d, J = 6.8 Hz, 4H), 1.10 (s, 3H), 0.86 - 0.79 |
| | (m, 1H), 0.76 (s, 3H). |
| 1898 | ¹H NMR (400 MHZ, CDC13) 8 8.71 (s, 1H), 8.13 - 8.08 (m, 1H), |
| | 7.98 (s, 1H), 7.91 - 7.86 (m, 1H), 7.85 (s, 1H), 7.69 (t, J = 7.8 |
| | Hz, 1H), 7.20 (t, J = 7.6 Hz, 1H), 7.06 (d, J = 7.6 Hz, 2H), 6.11 |
| | (s, 1H), 5.40 (dd, J = 11.3, 4.0 Hz, 1H), 5.08 (d, J = 15.5 Hz, |
| | 1H), 4.69 - 4.61 (m, 1H), 4.28 - 4.19 (m, 1H), 4.14 (t, J = 11.3 |
| | Hz, 1H), 4.01 - 3.91 (m, 2H), 3.06 - 2.94 (m, 1H), 2.70 (h, J = |
| | 8.8 Hz, 1H), 2.02 (s, 6H), 1.87 - 1.73 (m, 7H), 1.24 (d, J = 6.8 |
| | Hz, 3H), 1.17 (t, J = 10.3 Hz, 2H), 1.10 (s, 3H), 0.86 - 0.79 (m, |
| | 1H), 0.76 (s, 3H). |
| 1339 | ¹H NMR (400 MHZ, Chloroform-d) 8 8.75 (s, 1H), 8.04 (d, J = |
| | 7.9 Hz, 1H), 7.89 (d, J = 11.5 Hz, 2H), 7.84 (d, J = 7.7 Hz, 1H), |
| | 7.64 (t, J = 7.8 Hz, 1H), 7.21 (t, J = 7.6 Hz, 1H), 7.06 (d, J = |
| | 7.7 Hz, 2H), 6.20 (s, 1H), 5.41 - 5.32 (m, 1H), 5.18 (d, J = 15.6 |
| | Hz, 1H), 4.55 (p, J = 8.6 Hz, 1H), 4.06 - 4.01 (m, 2H), 3.13 (s, |
| | 3H), 2.32 - 2.25 (m, 2H), 2.24 - 2.17 (m, 2H), 2.04 (s, 7H), 1.87 - |
| | 1.81 (m, 2H), 1.75 - 1.70 (m, 2H), 1.61 - 1.53 (m, 2H), 1.40 - |
| | 1.33 (m, 2H), 1.04 (s, 3H), 0.90 (s, 3H), 0.83 - 0.76 (m, 1H). |
| 1340 | ¹H NMR (400 MHZ, Chloroform-d) 8 8.75 (s, 1H), 8.04 (d, J = |
| | 7.9 Hz, 1H), 7.89 (d, J = 12.3 Hz, 2H), 7.83 (d, J = 7.7 Hz, 1H), |
| | 7.64 (t, J = 7.7 Hz, 1H), 7.21 (t, J = 7.6 Hz, 1H), 7.06 (d, J = |
| | 7.7 Hz, 2H), 6.20 (s, 1H), 5.41 - 5.31 (m, 1H), 5.18 (d, J = 15.6 |
| | Hz, 1H), 4.55 (p, J = 8.5 Hz, 1H), 4.06 - 4.01 (m, 2H), 3.13 (s, |
| | 3H), 2.32 - 2.25 (m, 2H), 2.24 - 2.16 (m, 2H), 2.04 (s, 7H), 1.86 - |
| | 1.79 (m, 2H), 1.76 - 1.69 (m, 2H), 1.62 - 1.53 (m, 2H), 1.40 - |
| | 1.32 (m, 2H), 1.05 (s, 3H), 0.90 (s, 3H), 0.80 (t, J = 10.9, 8.6 |
| | Hz, 1H). |
| 1903 | ¹H NMR (400 MHz, Chloroform-d) 8 8.75 (s, 1H), 7.95 (d, J = |
| | 7.8 Hz, 1H), 7.88 (t, J = 8.2 Hz, 3H), 7.63 (t, J = 7.7 Hz, 1H), |
| | 7.21 (t, J = 7.6 Hz, 1H), 7.08 (d, J = 7.6 Hz, 2H), 6.26 (d, J = |
| | 2.0 Hz, 1H), 5.37 (dd, J = 10.7, 3.9 Hz, 1H), 5.13 (d, J = 15.6 |
| | Hz, 1H), 4.42 - 4.23 (m, 3H), 3.74 - 3.67 (m, 1H), 3.65 (dd, J = |
| | 10.8, 3.1 Hz, 1H), 3.34 (dd, J = 7.4, 3.3 Hz, 2H), 3.24 - 3.12 |
| | (m, 4H), 2.07 (s, 6H), 0.91 (dd, J = 6.7, 3.9 Hz, 6H), 0.56 - 0.38 |
| | (m, 4H). Sulfonamide NH not visible. Single H from isobutyl |
| | group likely under water peak. |
| 1904 | ¹H NMR (400 MHZ, Chloroform-d) 8 8.76 (s, 1H), 7.90 (d, J = |
| | 7.1 Hz, 2H), 7.84 (t, J = 8.8 Hz, 2H), 7.60 (t, J = 7.7 Hz, 1H), |
| | 7.25 - 7.19 (m, 1H), 7.09 (d, J = 7.6 Hz, 2H), 6.25 (s, 1H), 5.38 |
| | (dd, J = 10.3, 3.5 Hz, 1H), 5.15 (d, J = 15.6 Hz, 1H), 4.90 (p, J = |
| | 8.0 Hz, 1H), 4.38 - 4.25 (m, 3H), 3.73 - 3.62 (m, 2H), 3.19 |
| | (s, 3H), 3.18 - 3.14 (m, 1H), 2.51 (td, J = 13.2, 12.7, 8.0 Hz, |
| | 2H), 2.27 (dd, J = 11.9, 8.0 Hz, 2H), 2.07 (s, 6H), 0.51 - 0.38 |
| | (m, 8H) (sulfonamide NH not visible) |
| 1341 | ¹H NMR (400 MHZ, Chloroform-d) 8 8.71 (s, 1H), 7.90 - 7.77 |
| | (m, 4H), 7.59 (t, J = 7.7 Hz, 1H), 7.22 (d, J = 7.5 Hz, 1H), 7.09 |
| | (d, J = 7.6 Hz, 2H), 6.26 (s, 1H), 5.40 (d, J = 6.3 Hz, 1H), 5.10 |
| | (d, J = 15.4 Hz, 1H), 4.35 (d, J = 6.0 Hz, 2H), 4.26 (d, J = 15.2 |
| | Hz, 1H), 4.06 (q, J = 5.3 Hz, 1H), 3.73 - 3.64 (m, 2H), 3.62 - |
| | 3.54 (m, 2H), 3.23 - 3.16 (m, 1H), 2.40 - 2.29 (m, 1H), 2.08 (s, |
| | 6H), 2.04 - 1.92 (m, 4H), 0.91 (d, J = 6.9 Hz, 3H), 0.81 (d, J = |
| | 6.8 Hz, 3H), 0.53 - 0.41 (m, 4H). (Sulfonamide NH not visible) |
| 1906 | ¹H NMR (400 MHZ, Chloroform-d) 8 8.75 (s, 1H), 7.99 - 7.90 |
| | (m, 3H), 7.87 (d, J = 7.6 Hz, 1H), 7.64 (t, J = 7.7 Hz, 1H), 7.25 - |
| | 7.20 (m, 1H), 7.08 (d, J = 7.6 Hz, 2H), 6.26 (s, 1H), 5.32 (dd, |
| | J = 11.2, 4.4 Hz, 1H), 5.18 (d, J = 15.8 Hz, 1H), 4.92 (p, J = |
| | 6.6 Hz, 1H), 4.78 (p, J = 6.7 Hz, 1H), 4.37 - 4.29 (m, 2H), 4.21 |
| | (t, J = 11.5 Hz, 1H), 3.73 - 3.61 (m, 2H), 3.21 - 3.15 (m, 1H), |
| | 3.12 (s, 3H), 2.90 - 2.75 (m, 2H), 2.49 - 2.30 (m, 2H), 2.07 (s, |
| | 6H), 0.52 - 0.39 (m, 4H). Sulfonamide NH not visible |
| 1907 | ¹H NMR (400 MHz, Chloroform-d) 8 8.74 (d, J = 5.9 Hz, 1H), |
| | 7.94 (d, J = 24.5 Hz, 2H), 7.90 - 7.84 (m, 2H), 7.61 (t, J = 7.8 |
| | Hz, 1H), 7.22 (d, J = 7.6 Hz, 1H), 7.08 (d, J = 7.6 Hz, 2H), 6.26 |
| | (s, 1H), 5.36 (dd, J = 10.4, 3.6 Hz, 1H), 5.30 - 5.19 (m, 1H), |
| | 5.13 (d, J = 15.7 Hz, 1H), 5.03 (p, 1H), 4.40 - 4.25 (m, 3H), |
| | 3.73 - 3.63 (m, 2H), 3.19 - 3.14 (m, 1H), 3.12 (s, 3H), 2.69 - |
| | 2.47 (m, 4H), 2.08 (s, 6H), 0.50 - 0.37 (m, 4H). Sulfonamide |
| | NH not visible |
| 1908 | ¹H NMR (400 MHZ, CDC13) 8 8.71 (s, 1H), 7.98 (s, 1H), 7.87 |
| | (t, 3H), 7.64 - 7.57 (m, 1H), 7.25 - 7.19 (m, 1H), 7.09 (d, J = |
| | 7.6 Hz, 2H), 6.27 - 6.24 (m, 1H), 5.36 - 5.29 (m, 1H), 5.10 (d, |
| | J = 15.6 Hz, 1H), 4.69 - 4.61 (m, 1H), 4.40 - 4.18 (m, 5H), 3.73 - |
| | 3.60 (m, 3H), 3.19 - 3.12 (m, 1H), 3.04 - 2.94 (m, 1H), 2.08 |
| | (s, 6H), 1.34 - 1.28 (m, 3H), 1.22 (d, J = 6.7 Hz, 4H), 0.47 - |
| | 0.39 (m, 4H). |
| 1909 | ¹H NMR (400 MHZ, Chloroform-d) 8 8.80 (s, 1H), 7.93 - 7.79 |
| | (m, 2H), 7.74 (d, J = 7.6 Hz, 1H), 7.69 - 7.54 (m, 2H), 7.26 - |
| | 7.23 (m, 1H), 7.11 (d, J = 7.6 Hz, 2H), 6.28 (s, 1H), 5.47 (d, J = |
| | 9.3 Hz, 1H), 5.10 (d, J = 14.5 Hz, 1H), 4.44 - 4.27 (m, 3H), |
| | 4.07 (s, 1H), 3.79 (q, J = 9.3 Hz, 1H), 3.68 (s, 2H), 3.38 - 3.25 |
| | (m, 1H), 3.20 (s, 1H), 2.45 (s, 1H), 2.20 (q, J = 8.8 Hz, 1H), |
| | 2.11 (s, 6H), 2.09 - 2.03 (m, 1H), 2.00 - 1.94 (m, 1H), 1.83 - |
| | 1.70 (m, 3H), 0.55 - 0.41 (m, 4H). (Sulfonamide -NH not |
| | visible) |
| 1910 | ¹H NMR (400 MHZ, DMSO-d6) & 12.88 (br. s, 1H), 8.81 (s, |
| | 1H), 8.43 (s, 1H), 7.95 (d, J = 6.1 Hz, 1H), 7.74 - 7.61 (m, 2H), |
| | 7.31 - 7.22 (m, 1H), 7.17 - 7.08 (m, 2H), 6.43 (br. s, 1H), 6.40 |
| | (d, J = 0.7 Hz, 1H), 5.57 (dd, J = 10.5, 4.2 Hz, 1H), 5.02 (d, J = |
| | 16.1 Hz, 1H), 4.72 (d, J = 16.1 Hz, 1H), 4.47 - 4.23 (m, 3H), |
| | 4.12 - 4.00 (m, 1H), 2.53 (s, 3H), 2.07 (s, 6H), 1.82 (dd, J = |
| | 15.2, 8.8 Hz, 1H), 1.47 (d, J = 14.7 Hz, 1H), 1.27 (t, J = 7.1 |
| | Hz, 3H), 0.60 (s, 9H). |
| 1911 | ¹H NMR (400 MHZ, DMSO-d6) 8 12.98 (br. s, 1H), 8.82 (s, |
| | 1H), 8.44 (s, 1H), 7.94 (d, J = 4.9 Hz, 1H), 7.73 - 7.62 (m, 2H), |
| | 7.31 - 7.21 (m, 1H), 7.16 - 7.09 (m, 2H), 6.49 - 6.38 (m, 2H), |
| | 5.59 (dd, J = 10.6, 3.8 Hz, 1H), 5.04 (d, J = 15.9 Hz, 1H), 4.70 |
| | (d, J = 15.9 Hz, 1H), 4.30 (t, J = 11.0 Hz, 1H), 4.09 - 3.97 (m, |
| | 1H), 3.82 (s, 3H), 2.88 (q, J = 7.3 Hz, 2H), 2.23 - 1.88 (m, 6H), |
| | 1.83 (dd, J = 15.2, 8.8 Hz, 1H), 1.48 (d, J = 14.9 Hz, 1H), 1.32 |
| | (t, J = 7.5 Hz, 3H), 0.61 (s, 9H). |
| 1912 | ¹H NMR (400 MHZ, Chloroform-d) 8 8.76 (s, 1H), 7.94 - 7.80 |
| | (m, 4H), 7.62 (t, J = 7.7 Hz, 1H), 7.25 - 7.20 (m, 1H), 7.09 (d, |
| | J = 7.6 Hz, 2H), 6.27 (s, 1H), 5.37 (dd, J = 10.8, 4.1 Hz, 1H), |
| | 5.11 (d, J = 15.6 Hz, 1H), 4.41 - 4.24 (m, 4H), 3.98 - 3.89 (m, |
| | 1H), 3.73 - 3.61 (m, 2H), 3.32 (t, J = 10.7 Hz, 1H), 3.20 - 3.15 |
| | (m, 1H), 2.75 - 2.66 (m, 1H), 2.43 - 2.34 (m, 1H), 2.08 (s, 6H), |
| | 2.04 - 1.90 (m, 4H), 1.70 - 1.62 (m, 3H), 0.52 - 0.37 (m, 4H). |
| | (Sulfonamide NH not visible) |
| 1917 | ¹H NMR (400 MHZ, CDC13) 8 8.79 (s, 1H), 8.04 (d, J = 7.9 Hz, |
| | 1H), 7.87 (d, J = 7.4 Hz, 2H), 7.79 (s, 1H), 7.66 (t, J = 7.7 Hz, |
| | 1H), 7.21 (t, J = 7.6 Hz, 1H), 7.06 (d, J = 7.6 Hz, 2H), 6.24 (s, |
| | 1H), 5.42 (d, J = 6.9 Hz, 1H), 5.15 (d, J = 15.2 Hz, 1H), 4.23 - |
| | 3.97 (m, 5H), 2.21 - 2.10 (m, 2H), 2.04 (s, 6H), 1.79 (d, J = 6.1 |
| | Hz, 2H), 1.61 (s, 3H), 1.37 (d, J = 6.0 Hz, 3H), 1.30 (d, J = 6.0 |
| | Hz, 3H), 1.15 (d, J = 10.8 Hz, 1H), 0.81 (s, 9H). |
| 1918 | ¹H NMR (400 MHZ, CDC13) 8 8.68 (s, 1H), 7.92 (s, 1H), 7.81 - |
| | 7.78 (m, 1H), 7.65 (s, 1H), 7.51 (d, J = 4.7 Hz, 2H), 7.25 - 7.20 |
| | (m, 1H), 7.10 (d, J = 7.3 Hz, 2H), 6.24 (s, 1H), 5.41 (d, J = 6.4 |
| | Hz, 1H), 5.05 (d, J = 15.5 Hz, 1H), 4.50 - 4.44 (m, 1H), 4.33 - |
| | 4.28 (m, 2H), 4.15 - 4.08 (m, 1H), 4.01 - 3.93 (m, 1H), 3.69 - |
| | 3.64 (m, 2H), 3.20 - 3.15 (m, 1H), 2.58 - 2.48 (m, 1H), 2.11 - |
| | 2.08 (m, 6H), 1.55 (d, J = 6.2 Hz, 4H), 0.52 - 0.40 (m, 5H). |
| 1924 | ¹H NMR (500 MHZ, Chloroform-d) 8 8.40 (d, J = 7.9 Hz, 1H), |
| | 8.08 (t, J = 7.8 Hz, 1H), 7.94 - 7.85 (m, 3H), 7.26 - 7.21 (m, |
| | 1H), 7.20 (t, J = 7.7 Hz, 1H), 7.02 (d, J = 7.6 Hz, 2H), 6.07 (s, |
| | 1H), 5.85 - 5.78 (m, 1H), 5.23 (d, J = 15.9 Hz, 1H), 4.26 (d, J = |
| | 16.0 Hz, 1H), 4.17 - 4.08 (m, 1H), 4.10 - 3.98 (m, 1H), 3.72 - |
| | 3.64 (m, 1H), 2.87 (s, 3H), 2.35 - 2.26 (m, 1H), 2.25 - 2.15 |
| | (m, 1H), 2.16 - 2.04 (m, 1H), 2.06 - 1.89 (m, 7H), 1.82 - 1.73 |
| | (m, 1H), 1.76 - 1.62 (m, 2H), 1.56 - 1.49 (m, 1H), 0.64 (s, 9H). |
| 1925 | ¹H NMR (400 MHZ, CDC13) 8 8.75 - 8.72 (m, 1H), 7.89 - 7.82 |
| | (m, 4H), 7.64 - 7.57 (m, 1H), 7.24 - 7.19 (m, 1H), 7.08 (d, J = |
| | 7.6 Hz, 2H), 6.25 (s, 1H), 5.36 - 5.30 (m, 1H), 5.14 (d, J = 15.6 |
| | Hz, 1H), 4.47 - 4.33 (m, 3H), 4.31 - 4.24 (m, 2H), 3.73 - 3.61 |
| | (m, 5H), 3.19 - 3.13 (m, 1H), 2.37 - 2.30 (m, 3H), 2.19 - 2.12 |
| | (m, 3H), 2.07 (s, 6H), 0.47 - 0.40 (m, 5H). |
| 1926 | ¹H NMR (400 MHZ, CDC13) 8 8.71 - 8.68 (m, 1H), 7.93 (s, 1H), |
| | 7.82 - 7.78 (m, 1H), 7.72 - 7.67 (m, 1H), 7.65 (s, 1H), 7.55 (t, J = |
| | 7.7 Hz, 1H), 7.23 (d, J = 7.6 Hz, 1H), 7.09 (d, J = 7.6 Hz, |
| | 2H), 6.17 (s, 1H), 5.43 (dd, J = 10.7, 3.8 Hz, 1H), 5.09 (d, J = |
| | 15.5 Hz, 1H), 4.51 - 4.45 (m, 1H), 4.36 - 4.30 (m, 2H), 4.16 - |
| | 4.09 (m, 1H), 4.01 - 3.93 (m, 1H), 3.58 (d, J = 5.0 Hz, 2H), |
| | 3.50 - 3.40 (m, 3H), 2.58 - 2.48 (m, 1H), 2.10 (s, 6H), 1.55 (d, |
| | J = 6.2 Hz, 3H), 1.12 (d, J = 6.1 Hz, 3H), 1.06 (d, J = 6.1 Hz, |
| | 3H). |
| 1927 | ¹H NMR (400 MHZ, CDC13) 8 8.72 (s, 1H), 8.11 (br. s, 1H), |
| | 8.04 - 7.92 (m, 2H), 7.84 (br. s, 1H), 7.61 (br. s, 1H), 7.24 - 7.02 |
| | (m, 4H), 6.17 (s, 1H), 5.28 (d, J = 15.4 Hz, 1H), 5.19 - 5.08 (m, |
| | 1H), 4.28 - 4.07 (m, 2H), 4.06 - 3.84 (m, 2H), 2.87 (s, 3H), 2.35 - |
| | 2.20 (m, 2H), 2.17 - 1.97 (m, 8H), 1.83 (dd, J = 15.0, 8.7 Hz, |
| | 1H), 1.72 (td, J = 9.7, 5.6 Hz, 2H), 1.54 (d, J = 14.9 Hz, 1H), |
| | 0.63 (s, 9H), (1H missing, labile). |
| 1928 | ¹H NMR (400 MHZ, DMSO-d6) 8 13.13 (br. s., 1H), 11.44 (s, |
| | 1H), 8.66 (s, 1H), 8.02 (br. s., 1H), 7.74 (br. s., 2H), 7.47 (br. |
| | s., 2H), 7.27 (t, J = 7.3 Hz, 1H), 7.17 - 7.09 (m, 2H), 6.48 (br. |
| | s., 1H), 6.35 (s, 1H), 5.85 (s, 1H), 5.22 (dd, J = 10.3, 3.9 Hz, |
| | 1H), 4.80 - 4.65 (m, 2H), 4.36 (t, J = 10.9 Hz, 1H), 4.31 - 4.21 |
| | (m, 1H), 4.11 - 3.99 (m, 1H), 3.02 (s, 3H), 2.31 - 1.84 (m, 10H), |
| | 1.78 - 1.62 (m, 3H), 1.39 (d, J = 15.2 Hz, 1H), 0.46 (s, 9H). |
| 1929 | ¹H NMR (400 MHZ, DMSO-d6) 8 13.10 (br. s, 1H), 9.53 (s, |
| | 1H), 8.69 (br. s., 1H), 7.95 (br. s., 1H), 7.68 (br. s., 2H), 7.26 (t, |
| | J = 7.5 Hz, 1H), 7.12 (d, J = 6.1 Hz, 2H), 6.79 (s, 1H), 6.65 (d, |
| | J = 1.0 Hz, 1H), 6.42 (br. s., 1H), 5.38 (dd, J = 11.4, 3.8 Hz, |
| | 1H), 4.71 - 4.56 (m, 2H), 4.29 (d, J = 15.9 Hz, 1H), 4.19 (t, J = |
| | 10.8 Hz, 1H), 4.10 - 4.00 (m, 1H), 2.97 (s, 3H), 2.22 - 1.88 (m, |
| | 10H), 1.76 (dd, J = 15.0, 8.7 Hz, 1H), 1.67 - 1.58 (m, 2H), 1.48 |
| | (s, 9H), 1.37 (d, J = 14.7 Hz, 1H), 0.54 (s, 9H). |
| 1930 | ¹H NMR (400 MHZ, CDC13) 8 8.94 (t, J = 1.8 Hz, 1H), 7.95 (d, |
| | J = 8.1 Hz, 1H), 7.84 - 7.78 (m, 1H), 7.49 (t, J = 7.7 Hz, 1H), |
| | 7.46 - 7.37 (m, 1H), 7.26 - 7.22 (m, 2H), 7.07 (d, J = 7.6 Hz, |
| | 2H), 6.18 (s, 1H), 5.55 - 5.46 (m, 2H), 4.58 (d, J = 16.0 Hz, |
| | 1H), 4.39 - 4.27 (m, 1H), 4.17 (t, J = 11.6 Hz, 1H), 3.90 (s, 3H), |
| | 3.69 - 3.59 (m, 2H), 3.53 (hept, J = 6.1 Hz, 1H), 3.24 (hept, J = |
| | 6.9 Hz, 1H), 2.06 (s, 6H), 1.46 (d, J = 6.8 Hz, 3H), 1.43 (d, J = |
| | 6.8 Hz, 3H), 1.15 (d, J = 6.1 Hz, 3H), 1.12 (d, J = 6.1 Hz, |
| | 3H). |
| 1931 | ¹H NMR (400 MHZ, CDC13) 8 8.91 (t, J = 1.8 Hz, 1H), 8.46 (s, |
| | 1H), 8.09 - 8.01 (m, 1H), 7.82 (dt, J = 7.7, 1.3 Hz, 1H), 7.65 (t, |
| | J = 7.8 Hz, 1H), 7.22 (t, J = 7.6 Hz, 1H), 7.06 (d, J = 7.7 Hz, |
| | 2H), 6.42 (d, J = 0.8 Hz, 1H), 6.16 (s, 1H), 5.55 (dd, J = 11.0, |
| | 3.9 Hz, 1H), 5.44 (d, J = 15.7 Hz, 1H), 4.34 (d, J = 15.7 Hz, |
| | 1H), 4.24 - 4.14 (m, 1H), 4.06 (t, J = 11.2 Hz, 1H), 3.90 (s, 3H), |
| | 3.82 - 3.70 (m, 2H), 3.29 (ddd, J = 11.2, 9.3, 3.8 Hz, 1H), 3.21 - |
| | 3.12 (m, 1H), 3.07 (dd, J = 11.2, 8.9 Hz, 1H), 2.04 (s, 6H), |
| | 1.67 (t, J = 12.1 Hz, 2H), 1.59 - 1.42 (m, 3H), 1.39 (d, J = 6.8 |
| | Hz, 3H), 1.37 (d, J = 6.8 Hz, 3H), 1.24 (d, J = 13.2 Hz, 1H), |
| | 0.74 - 0.63 (m, 1H). |
| 1932 | ¹H NMR (400 MHZ, CDC13) 8 8.86 (s, 1H), 8.67 (s, 1H), 7.98 |
| | (d, J = 7.9 Hz, 1H), 7.89 (d, J = 7.7 Hz, 1H), 7.69 (t, J = 7.8 |
| | Hz, 1H), 7.28 (t, J = 7.7 Hz, 3H), 7.12 (d, J = 7.7 Hz, 2H), 6.93 |
| | (s, 1H), 6.34 (s, 1H), 5.60 (d, J = 9.9 Hz, 1H), 5.49 (d, J = 15.5 |
| | Hz, 1H), 4.74 (d, J = 15.9 Hz, 1H), 4.31 (t, J = 11.1 Hz, 1H), |
| | 4.22 - 4.08 (m, 1H), 4.04 (s, 3H), 3.78 - 3.67 (m, 1H), 3.37 - |
| | 3.21 (m, 3H), 2.72 (dd, J = 11.2, 8.3 Hz, 1H), 2.16 (s, 6H), 1.86 - |
| | 1.65 (m, 3H), 1.64 - 1.48 (m, 3H), 1.43 (d, J = 6.8 Hz, 3H), |
| | 1.41 (d, J = 6.8 Hz, 3H), 1.32 - 1.21 (m, 1H). |
| 1933 | ¹H NMR (400 MHZ, CDC13) 8 8.81 (t, J = 1.9 Hz, 1H), 8.47 (s, |
| | 1H), 7.81 (d, J = 7.7 Hz, 1H), 7.53 (t, J = 7.7 Hz, 1H), 7.45 (d, |
| | J = 8.0 Hz, 1H), 7.28 (t, J = 7.5 Hz, 1H), 7.10 (d, J = 7.6 Hz, |
| | 2H), 6.65 (s, 1H), 6.24 (s, 1H), 5.47 (d, J = 6.1 Hz, 1H), 5.42 |
| | (d, J = 15.9 Hz, 1H), 4.77 (d, J = 16.0 Hz, 1H), 4.57 - 4.45 (m, |
| | 1H), 4.44 - 4.37 (m, 1H), 4.35 (d, J = 5.9 Hz, 2H), 3.71 - 3.58 |
| | (m, 2H), 3.52 (p, J = 6.1 Hz, 1H), 3.18 (p, J = 6.8 Hz, 1H), 2.09 |
| | (s, 6H), 1.46 - 1.37 (m, 9H), 1.15 (d, J = 6.1 Hz, 3H), 1.10 (d, |
| | J = 6.1 Hz, 3H). |
| 1934 | ¹H NMR (400 MHZ, CDC13) 8 8.94 (d, J = 1.8 Hz, 1H), 8.48 |
| | (s, 1H), 8.03 (d, J = 7.9 Hz, 1H), 7.83 (d, J = 7.6 Hz, 1H), 7.64 |
| | (t, J = 7.8 Hz, 1H), 7.21 (t, J = 7.6 Hz, 1H), 7.05 (d, J = 7.6 |
| | Hz, 2H), 6.44 (s, 1H), 6.19 (s, 1H), 5.51 (dd, J = 11.1, 4.0 Hz, |
| | 1H), 5.47 (d, J = 15.7 Hz, 1H), 4.33 (d, J = 15.7 Hz, 1H), 4.25 - |
| | 4.15 (m, 1H), 4.09 - 3.95 (m, 1H), 3.90 (s, 3H), 3.23 - 3.07 |
| | (m, 1H), 2.01 (s, 6H), 1.73 - 1.55 (m, 5H), 1.52 - 1.44 (m, 2H), |
| | 1.39 (d, J = 6.8 Hz, 3H), 1.37 (d, J = 6.8 Hz, 3H), 1.26 - 1.16 |
| | (m, 2H), 1.07 - 0.92 (m, 3H), 0.51 - 0.35 (m, 1H). |
| 1936 | ¹H NMR (400 MHZ, CDC13) 8 8.70 (s, 1H), 8.07 - 8.03 (m, 1H), |
| | 7.97 - 7.94 (m, 1H), 7.88 - 7.84 (m, 2H), 7.68 - 7.63 (m, 1H), |
| | 7.22 - 7.18 (m, 1H), 7.09 - 7.06 (m, 1H), 7.06 - 7.04 (m, 1H), |
| | 6.24 (s, 1H), 5.45 - 5.36 (m, 2H), 5.15 - 5.06 (m, 2H), 4.39 - |
| | 4.32 (m, 1H), 4.20 - 3.95 (m, 6H), 3.86 - 3.82 (m, 1H), 3.66 - |
| | 3.40 (m, 5H), 3.26 (s, 3H), 2.05 (s, 6H), 1.14 - 1.11 (m, 3H), |
| | 0.81 (s, 9H). |
| 1937 | ¹H NMR (400 MHZ, CDC13) 8 8.74 (s, 1H), 8.07 (d, J = 7.8 Hz, |
| | 1H), 7.98 (s, 1H), 7.95 (s, 1H), 7.87 (d, J = 7.6 Hz, 1H), 7.67 |
| | (t, J = 7.8 Hz, 1H), 7.20 (t, J = 7.6 Hz, 1H), 7.06 (d, J = 7.6 |
| | Hz, 2H), 6.24 (s, 1H), 5.40 (dd, J = 10.9, 3.6 Hz, 1H), 5.18 (d, |
| | J = 15.3 Hz, 1H), 4.23 - 3.97 (m, 5H), 3.74 - 3.59 (m, 4H), 3.32 |
| | (s, 3H), 2.11 - 1.98 (m, 12H), 1.10 (d, J = 6.2 Hz, 3H), 0.81 (s, |
| | 9H). |
| 1938 | ¹H NMR (400 MHZ, CDC13) 8 8.77 (s, 1H), 7.86 - 7.80 (m, 2H), |
| | 7.73 - 7.67 (m, 2H), 7.61 - 7.54 (m, 1H), 7.25 - 7.21 (m, 1H), |
| | 7.09 (d, J = 7.5 Hz, 2H), 6.26 (s, 1H), 5.46 - 5.41 (m, 1H), 5.16 - |
| | 5.07 (m, 1H), 4.50 - 4.20 (m, 6H), 3.69 - 3.43 (m, 7H), 2.09 |
| | (s, 6H), 1.29 - 1.26 (m, 3H), 1.13 (d, J = 5.7 Hz, 3H), 1.07 (d, |
| | J = 5.8 Hz, 3H) |
| 1946 | ¹H NMR (400 MHZ, CDC13) 8 8.72 (s, 1H), 8.01 (s, 1H), 7.94 |
| | (s, 1H), 7.90 - 7.84 (m, 2H), 7.62 (t, J = 7.8 Hz, 1H), 7.23 - 7.20 |
| | (m, 1H), 7.07 (d, J = 7.7 Hz, 2H), 6.23 (s, 1H), 5.32 - 5.28 (m, |
| | 1H), 5.20 (d, J = 15.7 Hz, 1H), 4.74 - 4.67 (m, 1H), 4.44 (d, J = |
| | 15.7 Hz, 1H), 4.34 - 4.29 (m, 2H), 3.59 - 3.55 (m, 2H), 3.45 |
| | (p, J = 6.1 Hz, 1H), 3.12 (s, 3H), 3.03 - 2.93 (m, 2H), 2.81 - |
| | 2.67 (m, 2H), 2.05 (s, 6H), 1.12 (d, J = 6.1 Hz, 3H), 1.05 (d, J = |
| | 6.1 Hz, 3H). |
| 1947 | ¹H NMR (400 MHZ, CDC13) 8 8.77 (s, 1H), 8.07 (d, J = 7.9 Hz, |
| | 1H), 7.90 - 7.85 (m, 2H), 7.76 (s, 1H), 7.67 (t, J = 7.8 Hz, 1H), |
| | 7.22 - 7.18 (m, 1H), 7.08 (d, J = 7.7 Hz, 2H), 6.28 (s, 1H), 5.50 - |
| | 5.43 (m, 1H), 5.03 (d, J = 14.9 Hz, 1H), 4.37 - 4.17 (m, 3H), |
| | 4.12 - 3.95 (m, 2H), 2.30 - 2.24 (m, 2H), 2.07 (s, 6H), 1.71 (d, |
| | J = 6.1 Hz, 3H), 1.27 - 1.12 (m, 8H), 0.80 (d, J = 1.2 Hz, 9H), |
| | 0.76 - 0.68 (m, 1H). |
| 1959 | ¹H NMR (400 MHZ, DMSO-d6) 8 12.77 (br. s, 1H), 8.61 (s, |
| | 1H), 8.53 (s, 2H), 7.90 (d, J = 6.1 Hz, 1H), 7.72 - 7.54 (m, 2H), |
| | 7.28 - 7.17 (m, 1H), 7.14 - 7.08 (m, 2H), 6.61 - 6.00 (m, 1H), |
| | 5.35 (dd, J = 10.5, 3.7 Hz, 1H), 4.88 (d, J = 16.6 Hz, 1H), 4.81 |
| | (spt, J = 6.1 Hz, 1H), 4.58 (d, J = 16.4 Hz, 1H), 4.25 - 4.08 (m, |
| | 1H), 4.02 - 3.88 (m, 1H), 2.03 (br. s, 6H), 1.78 - 1.63 (m, 1H), |
| | 1.55 - 1.43 (m, 1H), 1.30 (d, J = 6.1 Hz, 3H), 1.30 (d, J = 6.1 |
| | Hz, 3H), 1.24 - 1.00 (m, 2H), 0.71 (s, 9H). |
| 1960 | ¹H NMR (400 MHZ, CDC13) 8 8.72 (s, 1H), 8.07 (d, J = 7.9 Hz, |
| | 1H), 7.91 (s, 1H), 7.87 - 7.82 (m, 2H), 7.66 (t, J = 7.8 Hz, 1H), |
| | 7.20 (t, J = 7.6 Hz, 1H), 7.04 (d, J = 7.6 Hz, 2H), 6.20 (s, 1H), |
| | 5.43 - 5.38 (m, 1H), 5.15 (d, J = 15.4 Hz, 1H), 4.57 (p, J = 8.5 |
| | Hz, 1H), 4.16 - 3.97 (m, 3H), 3.13 (s, 3H), 2.24 - 2.14 (m, 2H), |
| | 2.03 - 1.97 (m, 8H), 1.66 - 1.58 (m, 2H), 1.20 - 1.11 (m, 8H), |
| | 0.82 (s, 9H). |
| 1961 | ¹H NMR (400 MHZ, Chloroform-d) 8 9.65 (s, 1H), 8.64 (t, J = |
| | 1.8 Hz, 1H), 8.43 (s, 1H), 8.16 (d, J = 7.8 Hz, 1H), 7.83 (dt, J = |
| | 7.8, 1.3 Hz, 1H), 7.68 (t, J = 7.8 Hz, 1H), 7.19 (t, J = 7.6 Hz, |
| | 1H), 7.02 (d, J = 7.6 Hz, 2H), 6.62 (d, J = 1.1 Hz, 1H), 6.19 (s, |
| | 1H), 5.41 - 5.27 (m, 2H), 4.36 - 4.18 (m, 2H), 4.10 - 3.98 (m, |
| | 1H), 3.23 - 3.08 (m, 1H), 1.98 (s, 6H), 1.79 - 1.60 (m, 2H), 1.40 |
| | (d, J = 6.9 Hz, 6H), 1.17 (td, J = 12.8, 4.7 Hz, 1H), 0.91 - 0.73 |
| | (m, 10H). |
| 1972 | ¹H NMR (400 MHZ, Chloroform-d) 8 9.31 (broad s, 1H), 8.68 |
| | (t, J = 1.8 Hz, 1H), 8.37 (s, 1H), 7.88 - 7.79 (m, 2H), 7.59 (t, J = |
| | 7.8 Hz, 1H), 7.23 (t, J = 7.6 Hz, 1H), 7.06 (d, J = 7.6 Hz, |
| | 2H), 6.62 (d, J = 1.1 Hz, 1H), 6.22 (s, 1H), 5.42 - 5.27 (m, 2H), |
| | 4.58 (d, J = 15.1 Hz, 1H), 4.48 (t, J = 11.6 Hz, 1H), 4.41 - 4.28 |
| | (m, 1H), 3.71 - 3.55 (m, 2H), 3.46 (hept, J = 6.0 Hz, 1H), 3.23 - |
| | 3.08 (m, 1H), 2.04 (s, 6H), 1.40 (d, J = 6.9 Hz, 6H), 1.11 (d, |
| | J = 6.1 Hz, 3H), 1.06 (d, J = 6.1 Hz, 3H). |

D. Biological Activity—Assay Procedures

1. HBE Assay

Ussing Chamber Assay of CFTR-mediated short-circuit currents

Ussing chamber experiments were performed using human bronchial epithelial (HIBE) cells derived from CF subjects heterozygous for F508del and a minimal function CFTR mutation (F508del/MIF-HIBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 KH₂PO₄, 3.3 KH₂PO₄, 1.2 MgCl2, 1.2 CaCl₂), 10 Glucose, 10 HEPES (pH adjusted to 7.4 with NaOH) and the apical solution contained (in mM) 145 NaGluconate, 1.2 MgCl2, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 1 μM Ivacaftor or were incubated with the corrector compounds at a single fixed concentration of 10 μM in combination with 1 μM Ivacaftor for 18-24 hours at 37° C. and in the presence of 20% human serum. The concentration of corrector compounds with 1 μM Ivacaftor during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S)-8-[3-(2-{Dispiro[2.0.2.1]heptan-7-yl}ethoxy)-1H-pyrazol-1-yl]-12,12-dimethyl-2?⁶-thia-3,9,11,18,23-pentaazatetracyclo[17.3.1.111,14.05,10]tetracosa-1(22),5,7,9,19(23),20-hexaene-2,2,4-trione, in combination with 18 μM Tezacaftor and 1 μM Ivacaftor.

2. HBE2 Assay

Ussing Chamber Assay of CFTR-Mediated Short-Circuit Currents

Ussing chamber experiments were performed using human bronchial epithelial (HBE) cells derived from CF subjects heterozygous for F508del and a minimal function CFTR mutation (F508del/MF-HBE) and cultured as previously described (Neuberger T, Burton B, Clark H, Van Goor F Methods Mol Biol 2011:741:39-54). After four days the apical media was removed, and the cells were grown at an air liquid interface for >14 days prior to use. This resulted in a monolayer of fully differentiated columnar cells that were ciliated, features that are characteristic of human bronchial airway epithelia.

To isolate the CFTR-mediated short-circuit (I_{SC}) current, F508del/MF-HBE grown on Costar® Snapwell™ cell culture inserts were mounted in an Ussing chamber and the transepithelial I_{SC} was measured under voltage-clamp recording conditions (V_{hold}=0 mV) at 37° C. The basolateral solution contained (in mM) 145 NaCl, 0.83 KH₂PO₄, 3.3 KH₂PO₄, 1.2 MgCl2, 1.2 CaCl₂), 10 Glucose, 10 HEPES (pH adjusted to 7.4 with NaOH) and the apical solution contained (in mM) 145 NaGluconate, 1.2 MgCl2, 1.2 CaCl₂), 10 glucose, 10 HEPES (pH adjusted to 7.4 with NaOH) and 30 μM amiloride to block the epithelial sodium channel. Forskolin (20 μM) was added to the apical surface to activate CFTR, followed by apical addition of a CFTR inhibitor cocktail consisting of BPO, GlyH-101, and CFTR inhibitor 172 (each at 20 μM final assay concentration) to specifically isolate CFTR currents. The CFTR-mediated I_{SC} (μA/cm²) for each condition was determined from the peak forskolin response to the steady-state current following inhibition.

Identification of Corrector Compounds

The activity of the CFTR corrector compounds on the CFTR-mediated I_{SC} was determined in Ussing chamber studies as described above. The F508del/MF-HBE cell cultures were either incubated with the corrector compounds at a range of concentrations in combination with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol or were incubated with the corrector compounds at a single fixed concentration of 1 and 3 μM in combination with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol for 18-24 hours at 37° C. and in the presence of 20% human serum. The concentration of corrector compounds with 44 nM (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol during the 18-24 hours incubations was kept constant throughout the Ussing chamber measurement of the CFTR-mediated I_{SC} to ensure compounds were present throughout the entire experiment. The efficacy and potency of the putative F508del correctors was compared to that of the known Vertex corrector, (14S)-8-[3-(2-{Dispiro[2.0.2.1]heptan-7-yl}ethoxy)-1H-pyrazol-1-yl]-12,12-dimethyl-2λ⁶-thia-3,9,11,18,23-pentaazatetracyclo[17.3.1.111,14.05,10]tetracosa-1(22),5,7,9,19(23),20-hexaene-2,2,4-trione, in combination with 18 M Tezacaftor and 1 μM Ivacaftor.

E. Biological Activity Data

The following table represents CFTR modulating activity for representative compounds of the invention generated using one or more of the assays disclosed herein (EC₅₀: +++ is <1 μM; ++ is 1-43 3M; + is 3-<30 μM; and ND is “not detected in this assay.” % Activity: +++ is >60+; ++ is 30-60%; + is <300).

TABLE 16
Bioactivity Data for Compounds 1295-1924

| | | | HBE | HBE | | HBE2 | HBE2 |
|---|---|---|---|---|---|---|---|
| | | HBE | Max | Activity | HBE2 | Max | Activity |
| | | EC₅₀ | activity | at 10 | EC₅₀ | activity | at 3 μM |
| No. | Structure | (μM) | (%) | μM (%) | (μM) | (%) | (%) |
| 1343 | 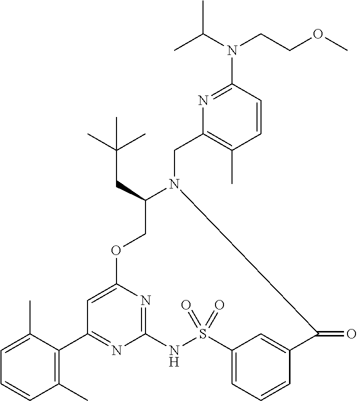 [SMILES: COCCN(c1ccc(C)c(CN2C(=O)c3cccc(c3)S(=O)(=O)Nc3nc(cc(-c4c(C)cccc4C)n3)OC[C@H]2CC(C)(C)C)n1)C(C)C] | +++ | +++ | | | | |
| 1344 | 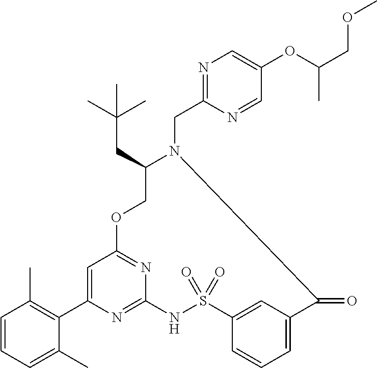 [SMILES: COCC(C)Oc1cnc(CN2C(=O)c3cccc(c3)S(=O)(=O)Nc3nc(cc(-c4c(C)cccc4C)n3)OC[C@H]2CC(C)(C)C)nc1] | +++ | +++ | | | | |
| 1345 | 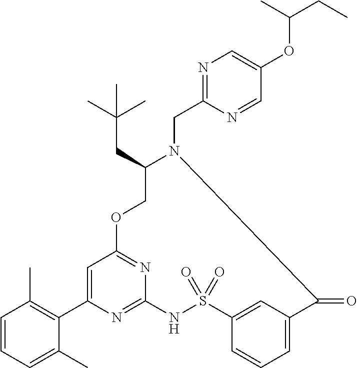 [SMILES: CCC(C)Oc1cnc(CN2C(=O)c3cccc(c3)S(=O)(=O)Nc3nc(cc(-c4c(C)cccc4C)n3)OC[C@H]2CC(C)(C)C)nc1] | +++ | +++ | | | | |
| 1346 | 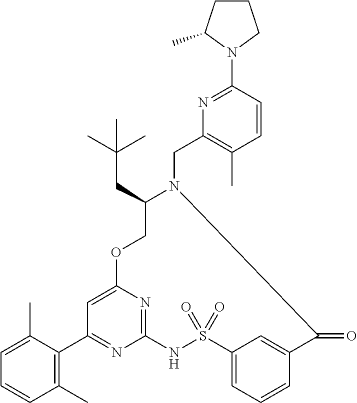 [SMILES: Cc1ccc(N2CCC[C@H]2C)nc1CN1C(=O)c2cccc(c2)S(=O)(=O)Nc2nc(cc(-c3c(C)cccc3C)n2)OC[C@H]1CC(C)(C)C] | +++ | +++ | | | | |
| 1347 | 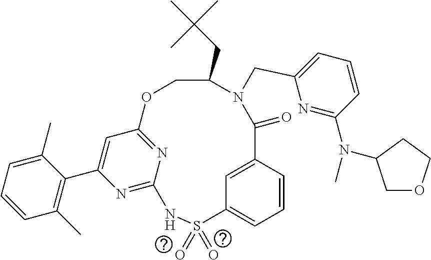 [SMILES: Cc1cccc(C)c1-c1cc2nc(n1)NS(=O)(=O)c1cccc(c1)C(=O)N(Cc1cccc(N(C)C3CCOC3)n1)[C@H](CC(C)(C)C)CO2] | +++ | +++ | | | | |
| 1348 | 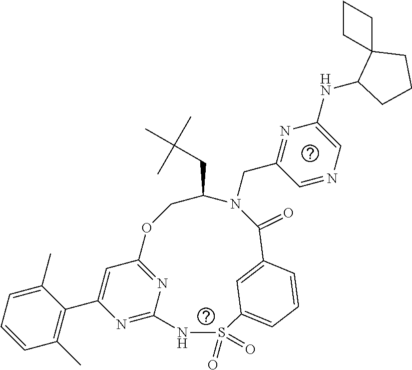 [SMILES: Cc1cccc(C)c1-c1cc2nc(n1)NS(=O)(=O)c1cccc(c1)C(=O)N(Cc1cncc(NC3CCCC34CCC4)n1)[C@H](CC(C)(C)C)CO2] | +++ | +++ | | | | |
| 1349 | 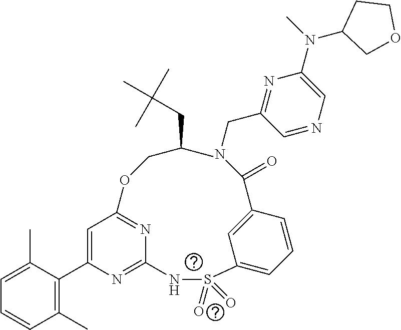 [SMILES: Cc1cccc(C)c1-c1cc(OC[C@H](C)CC(C)(C)C)nc(NS(=O)(=O)c2cccc(C(=O)NCc3cncc(N(C)C4CCOC4)n3)c2)n1] | +++ | +++ | | | | |
| 1350 | 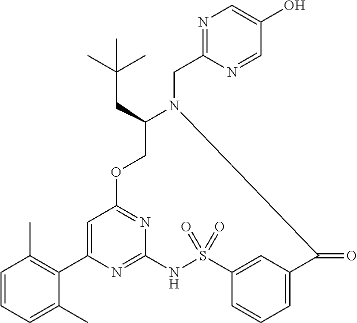 [SMILES: Cc1cccc(C)c1-c1cc2nc(n1)NS(=O)(=O)c1cccc(c1)C(=O)N(Cc1ncc(O)cn1)[C@H](CC(C)(C)C)CO2] | | | | | | |
| 1351 | 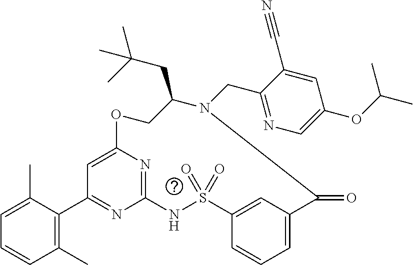 [SMILES: Cc1cccc(C)c1-c1cc2nc(n1)NS(=O)(=O)c1cccc(c1)C(=O)N(Cc1ncc(OC(C)C)cc1C#N)[C@H](CC(C)(C)C)CO2] | +++ | +++ | | | | |
| 1352 | 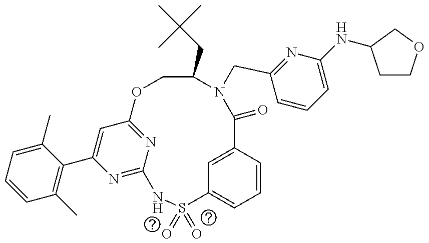 [SMILES: Cc1cccc(C)c1-c1cc2nc(n1)NS(=O)(=O)c1cccc(c1)C(=O)N(Cc1cccc(NC3CCOC3)n1)[C@H](CC(C)(C)C)CO2] | +++ | +++ | | | | |
| 1353 | 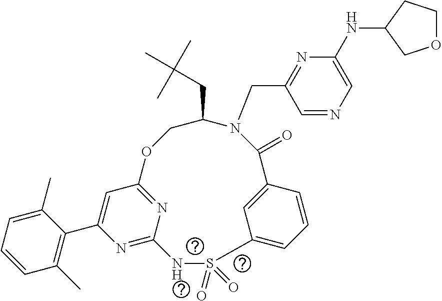 [SMILES: Cc1cccc(C)c1-c1cc2nc(n1)NS(=O)(=O)c1cccc(c1)C(=O)N(Cc1cncc(NC3CCOC3)n1)[C@H](CC(C)(C)C)CO2] | +++ | +++ | | | | |
| 1354 | 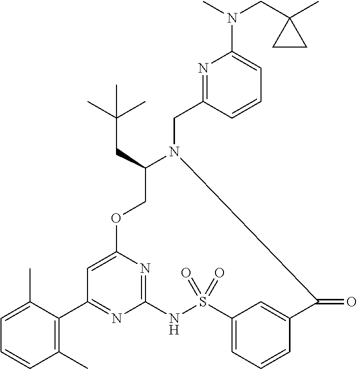 [SMILES: Cc1cccc(C)c1-c1cc2nc(n1)NS(=O)(=O)c1cccc(c1)C(=O)N(Cc1cccc(N(C)CC3(C)CC3)n1)[C@H](CC(C)(C)C)CO2] | +++ | +++ | | | | |
| 1355 | 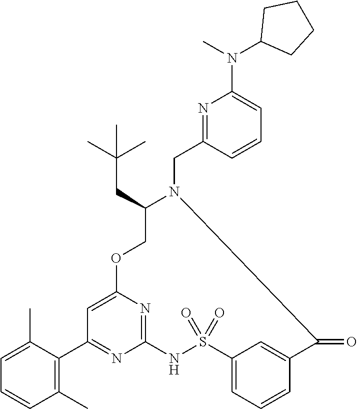 [SMILES: Cc1cccc(C)c1-c1cc2nc(n1)NS(=O)(=O)c1cccc(c1)C(=O)N(Cc1cccc(N(C)C3CCCC3)n1)[C@H](CC(C)(C)C)CO2] | +++ | +++ | | | | |
| 1356 | 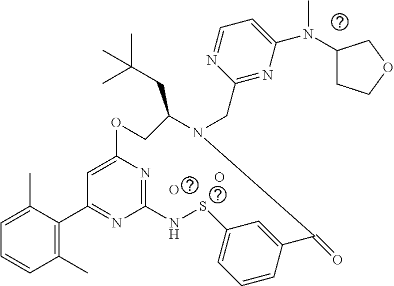 | | | | | | |
| 1357 | 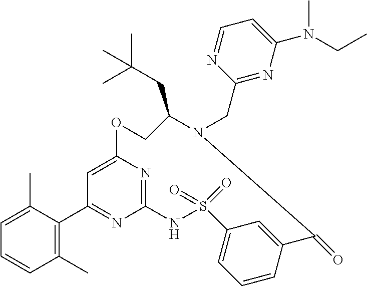 [SMILES: CCN(C)c1ccnc(CN2C(=O)c3cccc(c3)S(=O)(=O)Nc3nc(cc(-c4c(C)cccc4C)n3)OC[C@H]2CC(C)(C)C)n1] | +++ | +++ | | | | |
| 1358 | 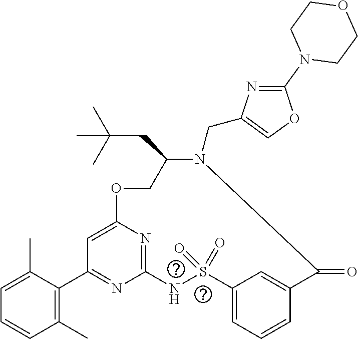 [SMILES: Cc1cccc(C)c1-c1cc2nc(n1)NS(=O)(=O)c1cccc(c1)C(=O)N(Cc1coc(N3CCOCC3)n1)[C@H](CC(C)(C)C)CO2] | +++ | +++ | | | | |
| 1358 | 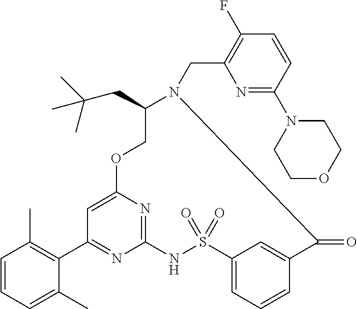 [SMILES: Cc1cccc(C)c1-c1cc2nc(n1)NS(=O)(=O)c1cccc(c1)C(=O)N(Cc1nc(N3CCOCC3)ccc1F)[C@H](CC(C)(C)C)CO2] | | | | | | |
| 1360 | 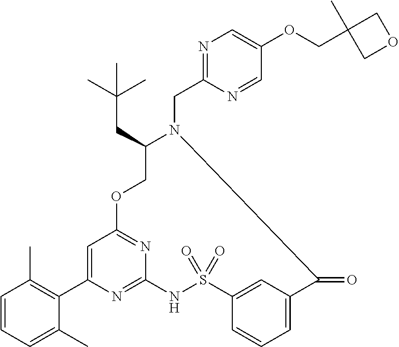 [SMILES: Cc1cccc(C)c1-c1cc2nc(n1)NS(=O)(=O)c1cccc(c1)C(=O)N(Cc1ncc(OCC3(C)COC3)cn1)[C@H](CC(C)(C)C)CO2] | +++ | +++ | | | | |
| 1361 | 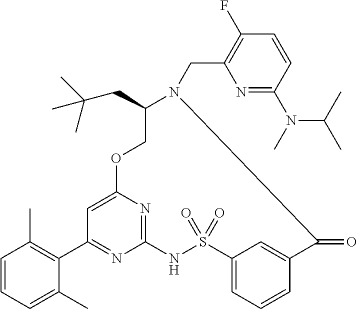 [SMILES: Cc1cccc(C)c1-c1cc2nc(n1)NS(=O)(=O)c1cccc(c1)C(=O)N(Cc1nc(N(C)C(C)C)ccc1F)[C@H](CC(C)(C)C)CO2] | | | | | | |
| 1362 | 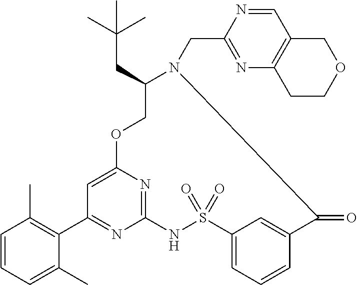 [SMILES: Cc1cccc(C)c1-c1cc2nc(n1)NS(=O)(=O)c1cccc(c1)C(=O)N(Cc1ncc3c(n1)CCOC3)[C@H](CC(C)(C)C)CO2] | | | +++ | | | |
| 1363 | 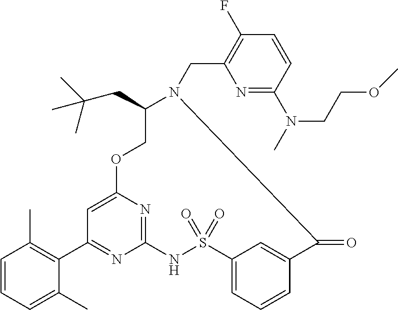 [SMILES: COCCN(C)c1ccc(F)c(CN2C(=O)c3cccc(c3)S(=O)(=O)Nc3nc(cc(-c4c(C)cccc4C)n3)OC[C@H]2CC(C)(C)C)n1] | | | | | | |
| 1364 | 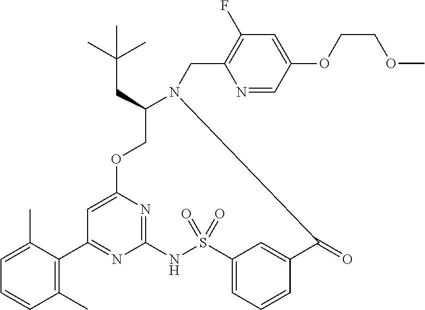 [SMILES: COCCOc1cnc(CN2C(=O)c3cccc(c3)S(=O)(=O)Nc3nc(cc(-c4c(C)cccc4C)n3)OC[C@H]2CC(C)(C)C)c(F)c1] | +++ | +++ | | | | |
| 1365 | 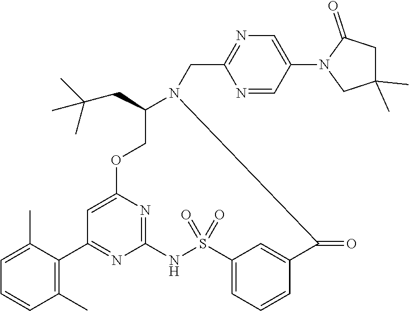 [SMILES: Cc1cccc(C)c1-c1cc2nc(n1)NS(=O)(=O)c1cccc(c1)C(=O)N(Cc1ncc(N3CC(C)(C)CC3=O)cn1)[C@H](CC(C)(C)C)CO2] | +++ | +++ | | | | |
| 1366 | 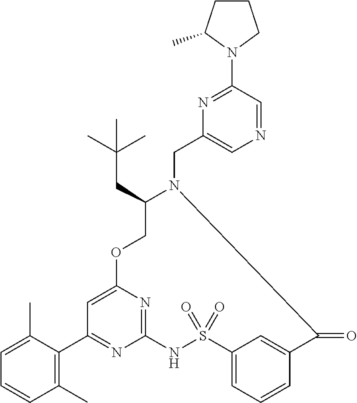 [SMILES: Cc1cccc(C)c1-c1cc2nc(n1)NS(=O)(=O)c1cccc(c1)C(=O)N(Cc1cncc(N3CCC[C@H]3C)n1)[C@H](CC(C)(C)C)CO2] | +++ | +++ | | | | |
| 1367 | 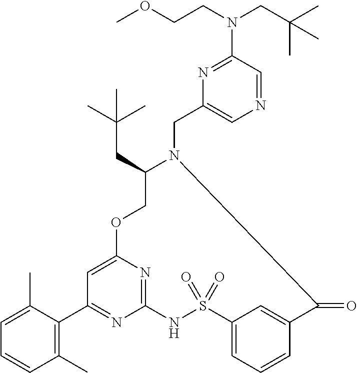 [SMILES: COCCN(CC(C)(C)C)c1cncc(CN2C(=O)c3cccc(c3)S(=O)(=O)Nc3nc(cc(-c4c(C)cccc4C)n3)OC[C@H]2CC(C)(C)C)n1] | +++ | +++ | | | | |
| 1368 | 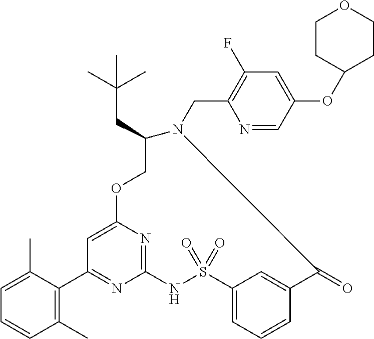 [SMILES: Cc1cccc(C)c1-c1cc2nc(n1)NS(=O)(=O)c1cccc(c1)C(=O)N(Cc1ncc(OC3CCOCC3)cc1F)[C@H](CC(C)(C)C)CO2] | +++ | +++ | | | | |
| 1369 | 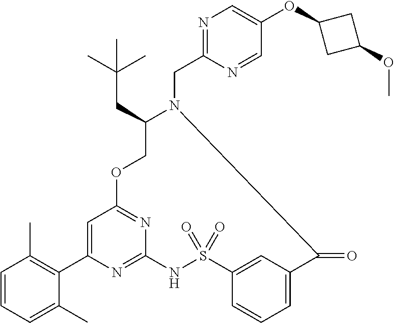 [SMILES: CO[C@H]1C[C@@H](Oc2cnc(CN3C(=O)c4cccc(c4)S(=O)(=O)Nc4nc(cc(-c5c(C)cccc5C)n4)OC[C@H]3CC(C)(C)C)nc2)C1] | +++ | +++ | | | | |
| 1370 | 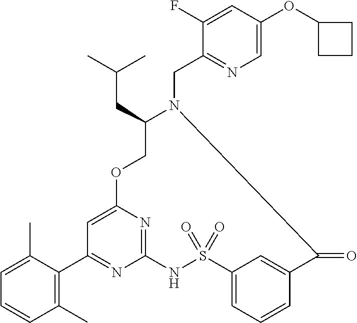 [SMILES: Cc1cccc(C)c1-c1cc2nc(n1)NS(=O)(=O)c1cccc(c1)C(=O)N(Cc1ncc(OC3CCC3)cc1F)[C@H](CC(C)C)CO2] | +++ | +++ | | | | |
| 1299 | 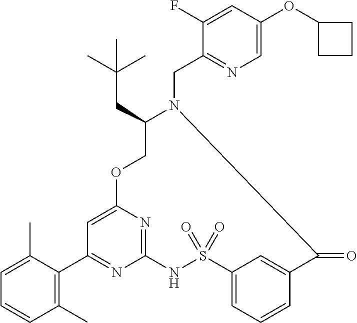 [SMILES: Cc1cccc(C)c1-c1cc2nc(n1)NS(=O)(=O)c1cccc(c1)C(=O)N(Cc1ncc(OC3CCC3)cc1F)[C@H](CC(C)(C)C)CO2] | +++ | +++ | | | | |
| 1371 | 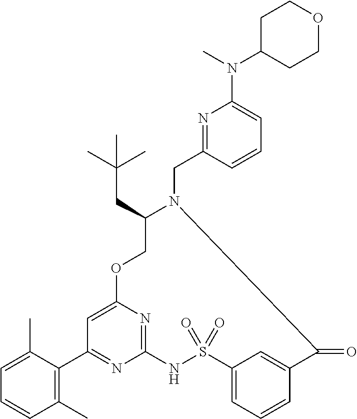 [SMILES: Cc1cccc(C)c1-c1cc2nc(n1)NS(=O)(=O)c1cccc(c1)C(=O)N(Cc1cccc(N(C)C3CCOCC3)n1)[C@H](CC(C)(C)C)CO2] | +++ | +++ | | | | |
| 1372 | 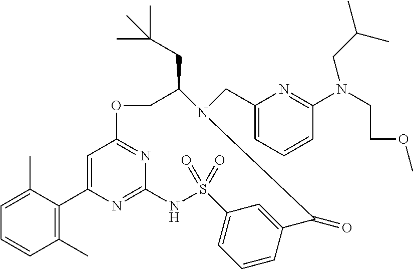 [SMILES: COCCN(CC(C)C)c1cccc(CN2C(=O)c3cccc(c3)S(=O)(=O)Nc3nc(cc(-c4c(C)cccc4C)n3)OC[C@H]2CC(C)(C)C)n1] | +++ | +++ | | | | |
| 1373 | 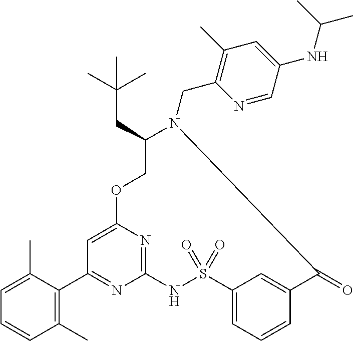 [SMILES: Cc1cc(NC(C)C)cnc1CN1C(=O)c2cccc(c2)S(=O)(=O)Nc2nc(cc(-c3c(C)cccc3C)n2)OC[C@H]1CC(C)(C)C] | +++ | +++ | | | | |
| 1374 | 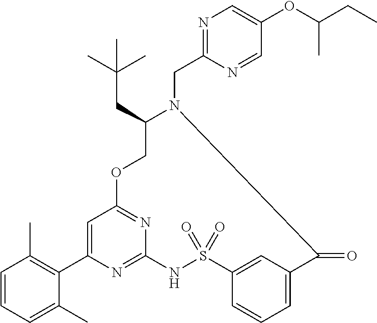 [SMILES: CCC(C)Oc1cnc(CN2C(=O)c3cccc(c3)S(=O)(=O)Nc3nc(cc(-c4c(C)cccc4C)n3)OC[C@H]2CC(C)(C)C)nc1] | +++ | +++ | | | | |
| 1375 | 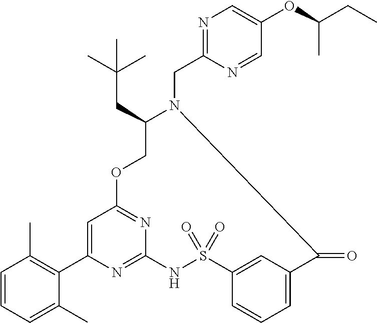 [SMILES: CC[C@@H](C)Oc1cnc(CN2C(=O)c3cccc(c3)S(=O)(=O)Nc3nc(cc(-c4c(C)cccc4C)n3)OC[C@H]2CC(C)(C)C)nc1] | +++ | +++ | | | | |
| 1376 | 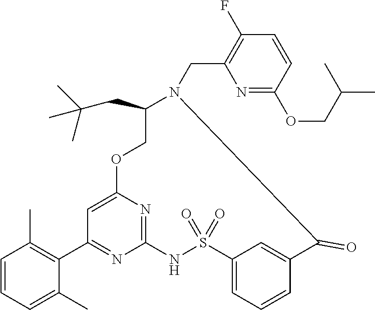 [SMILES: Cc1cccc(C)c1-c1cc2nc(n1)NS(=O)(=O)c1cccc(c1)C(=O)N(Cc1nc(OCC(C)C)ccc1F)[C@H](CC(C)(C)C)CO2] | +++ | +++ | | | | |
| 1300 | 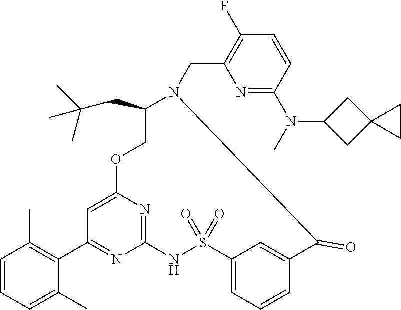 [SMILES: Cc1cccc(C)c1-c1cc2nc(n1)NS(=O)(=O)c1cccc(c1)C(=O)N(Cc1nc(N(C)C3CC4(CC4)C3)ccc1F)[C@H](CC(C)(C)C)CO2] | +++ | +++ | | | | |
| 1301 | 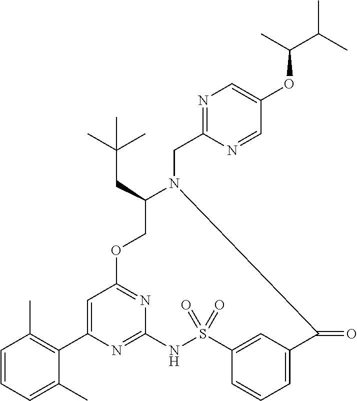 [SMILES: Cc1cccc(C)c1-c1cc2nc(n1)NS(=O)(=O)c1cccc(c1)C(=O)N(Cc1ncc(O[C@@H](C)C(C)C)cn1)[C@H](CC(C)(C)C)CO2] | +++ | +++ | | | | |
| 1377 | 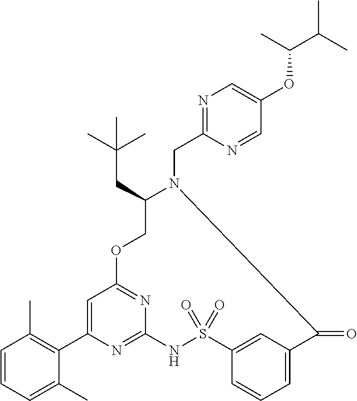 [SMILES: Cc1cccc(C)c1-c1cc2nc(n1)NS(=O)(=O)c1cccc(c1)C(=O)N(Cc1ncc(O[C@H](C)C(C)C)cn1)[C@H](CC(C)(C)C)CO2] | +++ | +++ | | | | |
| 1302 | 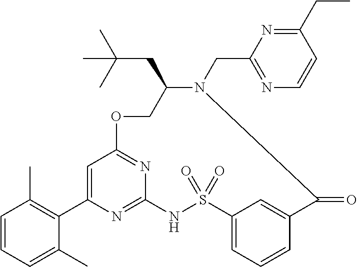 [SMILES: CCc1ccnc(CN2C(=O)c3cccc(c3)S(=O)(=O)Nc3nc(cc(-c4c(C)cccc4C)n3)OC[C@H]2CC(C)(C)C)n1] | | | | | | +++ |
| 1378 | 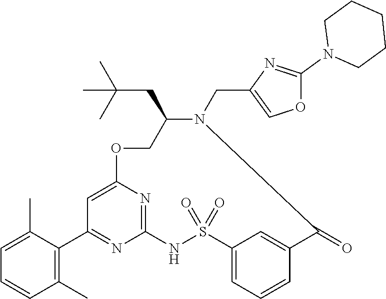 [SMILES: Cc1cccc(C)c1-c1cc2nc(n1)NS(=O)(=O)c1cccc(c1)C(=O)N(Cc1coc(N3CCCCC3)n1)[C@H](CC(C)(C)C)CO2] | | | | | | |
| 1379 | 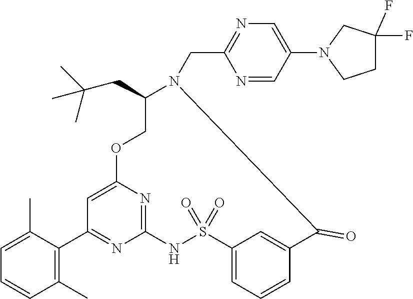 [SMILES: Cc1cccc(C)c1-c1cc2nc(n1)NS(=O)(=O)c1cccc(c1)C(=O)N(Cc1ncc(N3CCC(F)(F)C3)cn1)[C@H](CC(C)(C)C)CO2] | +++ | +++ | | | | |
| 1380 | 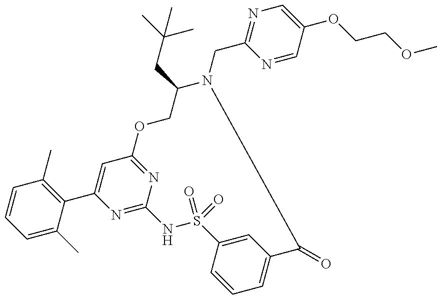 [SMILES: COCCOc1cnc(CN2C(=O)c3cccc(c3)S(=O)(=O)Nc3nc(cc(-c4c(C)cccc4C)n3)OC[C@H]2CC(C)(C)C)nc1] | +++ | +++ | | | | |
| 1381 | 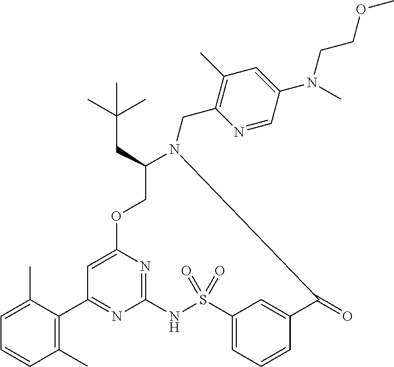 [SMILES: COCCN(C)c1cnc(CN2C(=O)c3cccc(c3)S(=O)(=O)Nc3nc(cc(-c4c(C)cccc4C)n3)OC[C@H]2CC(C)(C)C)c(C)c1] | +++ | +++ | | | | |
| 1382 | 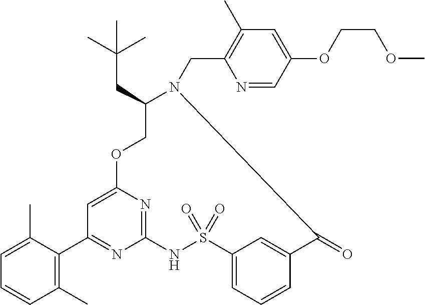 [SMILES: COCCOc1cnc(CN2C(=O)c3cccc(c3)S(=O)(=O)Nc3nc(cc(-c4c(C)cccc4C)n3)OC[C@H]2CC(C)(C)C)c(C)c1] | +++ | +++ | | | | |
| 1383 | 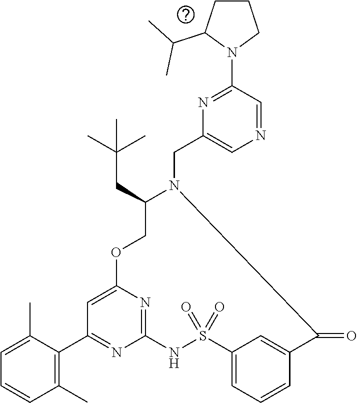 [SMILES: Cc1cccc(C)c1-c1cc2nc(n1)NS(=O)(=O)c1cccc(c1)C(=O)N(Cc1cncc(N3CCCC3C(C)C)n1)[C@H](CC(C)(C)C)CO2] | +++ | +++ | | | | |
| 1384 | 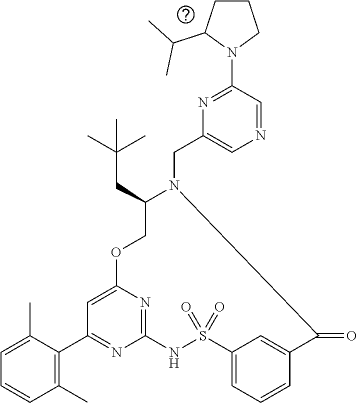 [SMILES: Cc1cccc(C)c1-c1cc2nc(n1)NS(=O)(=O)c1cccc(c1)C(=O)N(Cc1cncc(N3CCCC3C(C)C)n1)[C@H](CC(C)(C)C)CO2] | +++ | +++ | | | | |
| 1385 | 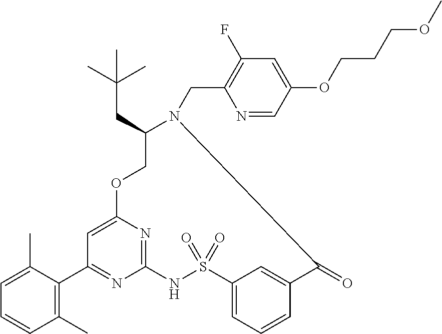 [SMILES: COCCCOc1cnc(CN2C(=O)c3cccc(c3)S(=O)(=O)Nc3nc(cc(-c4c(C)cccc4C)n3)OC[C@H]2CC(C)(C)C)c(F)c1] | +++ | +++ | | | | |
| 1386 | 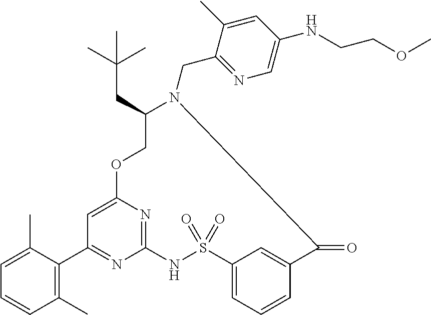 [SMILES: COCCNc1cnc(CN2C(=O)c3cccc(c3)S(=O)(=O)Nc3nc(cc(-c4c(C)cccc4C)n3)OC[C@H]2CC(C)(C)C)c(C)c1] | | | | | | |
| 1387 | 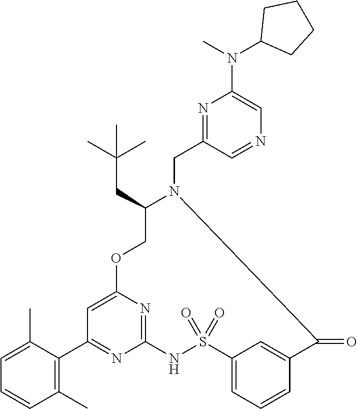 [SMILES: Cc1cccc(C)c1-c1cc2nc(n1)NS(=O)(=O)c1cccc(c1)C(=O)N(Cc1cncc(N(C)C3CCCC3)n1)[C@H](CC(C)(C)C)CO2] | +++ | +++ | | | | |
| 1388 | 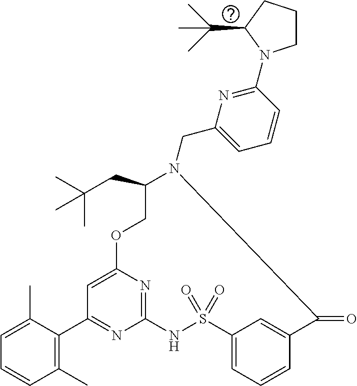 [SMILES: Cc1cccc(C)c1-c1cc2nc(n1)NS(=O)(=O)c1cccc(c1)C(=O)N(Cc1cccc(N3CCC[C@@H]3C(C)(C)C)n1)[C@H](CC(C)(C)C)CO2] | | | | | | |
| 1389 | 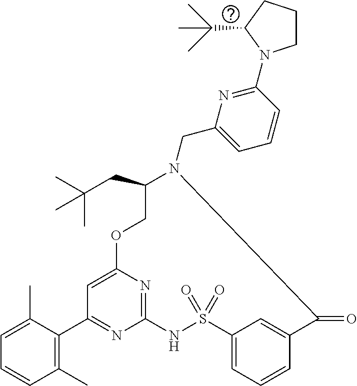 [SMILES: Cc1cccc(C)c1-c1cc2nc(n1)NS(=O)(=O)c1cccc(c1)C(=O)N(Cc1cccc(N3CCC[C@H]3C(C)(C)C)n1)[C@H](CC(C)(C)C)CO2] | | | | | | |
| 1390 | 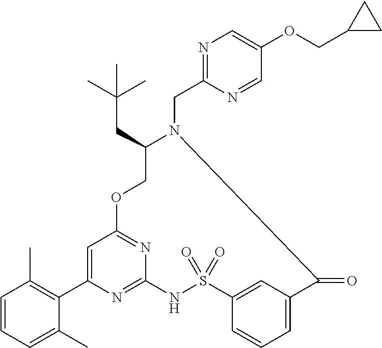 [SMILES: Cc1cccc(C)c1-c1cc2nc(n1)NS(=O)(=O)c1cccc(c1)C(=O)N(Cc1ncc(OCC3CC3)cn1)[C@H](CC(C)(C)C)CO2] | +++ | +++ | | | | |
| 1391 | 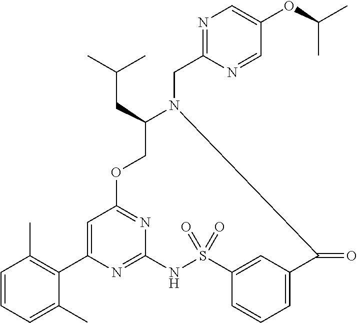 | +++ | +++ | | | | |
| 1392 | 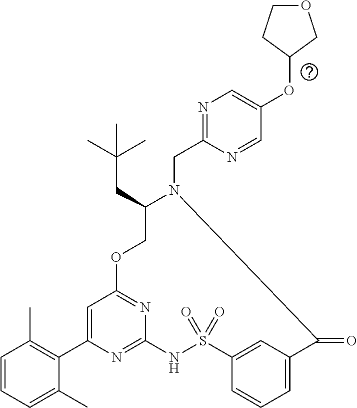 [SMILES: Cc1cccc(C)c1-c1cc2nc(n1)NS(=O)(=O)c1cccc(c1)C(=O)N(Cc1ncc(OC3CCOC3)cn1)[C@H](CC(C)(C)C)CO2] | | | | | | |
| 1393 | 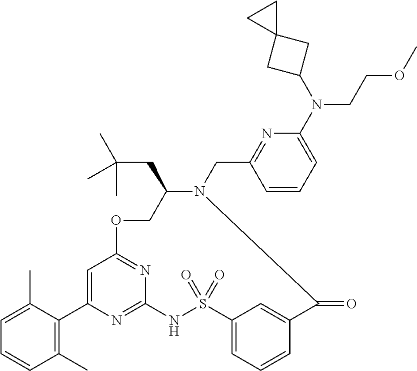 [SMILES: COCCN(c1cccc(CN2C(=O)c3cccc(c3)S(=O)(=O)Nc3nc(cc(-c4c(C)cccc4C)n3)OC[C@H]2CC(C)(C)C)n1)C1CC2(CC2)C1] | +++ | +++ | | | | |
| 1394 | 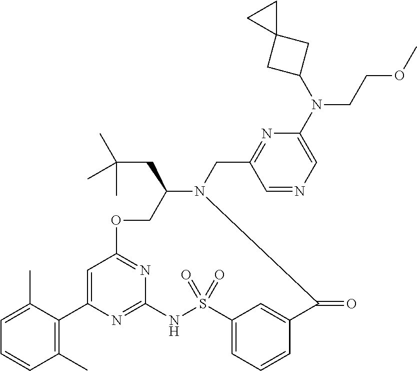 [SMILES: COCCN(c1cncc(CN2C(=O)c3cccc(c3)S(=O)(=O)Nc3nc(cc(-c4c(C)cccc4C)n3)OC[C@H]2CC(C)(C)C)n1)C1CC2(CC2)C1] | +++ | +++ | | | | |
| 1395 | 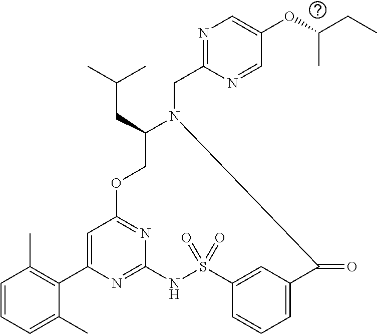 [SMILES: CC[C@H](C)Oc1cnc(CN2C(=O)c3cccc(c3)S(=O)(=O)Nc3nc(cc(-c4c(C)cccc4C)n3)OC[C@H]2CC(C)C)nc1] | +++ | +++ | | | | |
| 1396 | 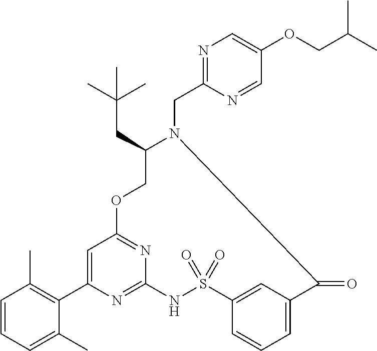 [SMILES: Cc1cccc(C)c1-c1cc2nc(n1)NS(=O)(=O)c1cccc(c1)C(=O)N(Cc1ncc(OCC(C)C)cn1)[C@H](CC(C)(C)C)CO2] | +++ | +++ | | | | |
| 1397 | 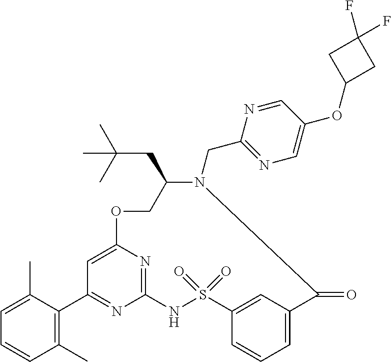 [SMILES: Cc1cccc(C)c1-c1cc2nc(n1)NS(=O)(=O)c1cccc(c1)C(=O)N(Cc1ncc(OC3CC(F)(F)C3)cn1)[C@H](CC(C)(C)C)CO2] | +++ | +++ | | | | |
| 1402 | 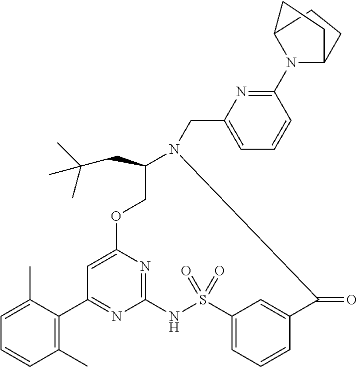 [SMILES: Cc1cccc(C)c1-c1cc2nc(n1)NS(=O)(=O)c1cccc(c1)C(=O)N(Cc1cccc(N3C4CCC3CC4)n1)[C@H](CC(C)(C)C)CO2] | +++ | +++ | | | | |
| 1399 | 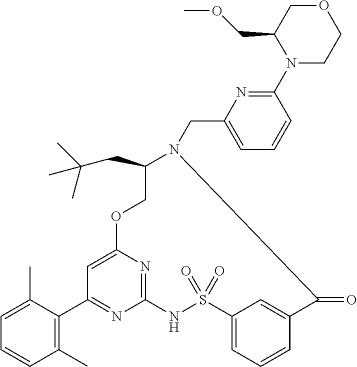 [SMILES: COC[C@H]1COCCN1c1cccc(CN2C(=O)c3cccc(c3)S(=O)(=O)Nc3nc(cc(-c4c(C)cccc4C)n3)OC[C@H]2CC(C)(C)C)n1] | | | +++ | | | |
| 1400 | 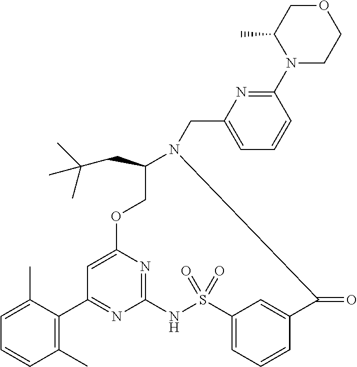 [SMILES: Cc1cccc(C)c1-c1cc2nc(n1)NS(=O)(=O)c1cccc(c1)C(=O)N(Cc1cccc(N3CCOC[C@H]3C)n1)[C@H](CC(C)(C)C)CO2] | +++ | +++ | | | | |
| 1401 | 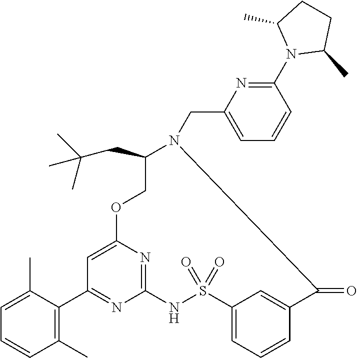 [SMILES: Cc1cccc(C)c1-c1cc2nc(n1)NS(=O)(=O)c1cccc(c1)C(=O)N(Cc1cccc(N3[C@H](C)CC[C@H]3C)n1)[C@H](CC(C)(C)C)CO2] | +++ | +++ | | | | |
| 1402 | 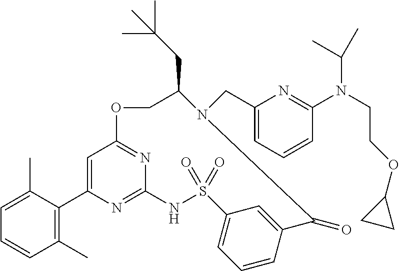 [SMILES: Cc1cccc(C)c1-c1cc2nc(n1)NS(=O)(=O)c1cccc(c1)C(=O)N(Cc1cccc(N(CCOC3CC3)C(C)C)n1)[C@H](CC(C)(C)C)CO2] | | | | | | |
| 1403 | 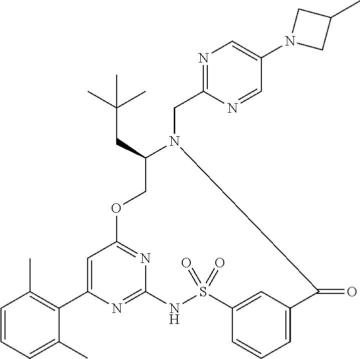 [SMILES: Cc1cccc(C)c1-c1cc2nc(n1)NS(=O)(=O)c1cccc(c1)C(=O)N(Cc1ncc(N3CC(C)C3)cn1)[C@H](CC(C)(C)C)CO2] | +++ | +++ | | | | |
| 1404 | 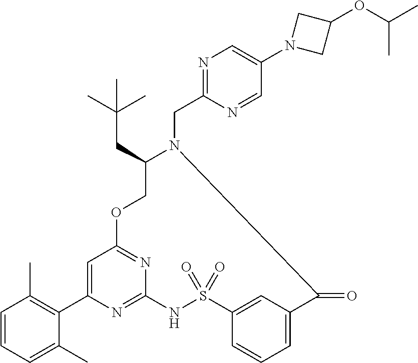 [SMILES: Cc1cccc(C)c1-c1cc2nc(n1)NS(=O)(=O)c1cccc(c1)C(=O)N(Cc1ncc(N3CC(OC(C)C)C3)cn1)[C@H](CC(C)(C)C)CO2] | +++ | +++ | | | | |
| 1405 | 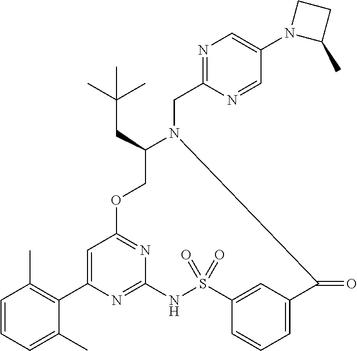 [SMILES: Cc1cccc(C)c1-c1cc2nc(n1)NS(=O)(=O)c1cccc(c1)C(=O)N(Cc1ncc(N3CC[C@H]3C)cn1)[C@H](CC(C)(C)C)CO2] | +++ | +++ | | | | |
| 1406 | 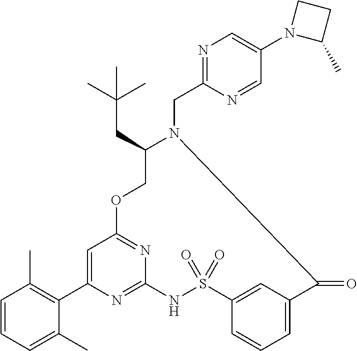 [SMILES: Cc1cccc(C)c1-c1cc2nc(n1)NS(=O)(=O)c1cccc(c1)C(=O)N(Cc1ncc(N3CC[C@@H]3C)cn1)[C@H](CC(C)(C)C)CO2] | +++ | +++ | | | | |
| 1407 | 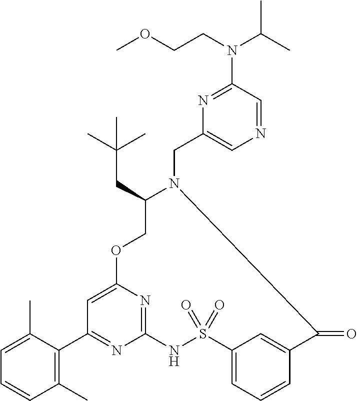 [SMILES: COCCN(c1cncc(CN2C(=O)c3cccc(c3)S(=O)(=O)Nc3nc(cc(-c4c(C)cccc4C)n3)OC[C@H]2CC(C)(C)C)n1)C(C)C] | +++ | +++ | | | | |
| 1408 | 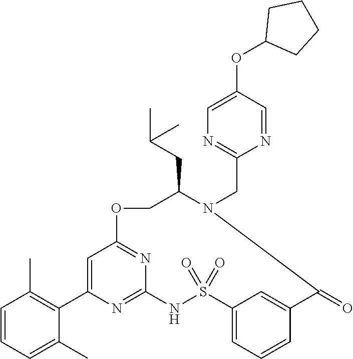 [SMILES: Cc1cccc(C)c1-c1cc2nc(n1)NS(=O)(=O)c1cccc(c1)C(=O)N(Cc1ncc(OC3CCCC3)cn1)[C@H](CC(C)C)CO2] | ++ | +++ | | | | |
| 1409 | 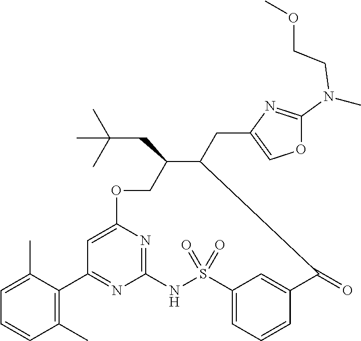 [SMILES: COCCN(C)c1nc(CC2C(=O)c3cccc(c3)S(=O)(=O)Nc3nc(cc(-c4c(C)cccc4C)n3)OC[C@H]2CC(C)(C)C)co1] | | | | | | +++ |
| 1410 | 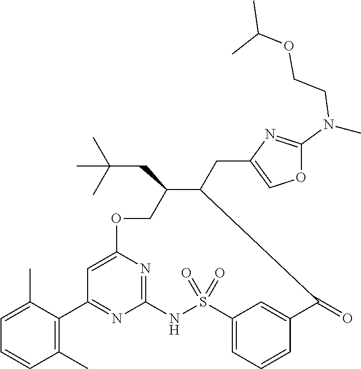 [SMILES: Cc1cccc(C)c1-c1cc2nc(n1)NS(=O)(=O)c1cccc(c1)C(=O)C(Cc1coc(N(C)CCOC(C)C)n1)[C@H](CC(C)(C)C)CO2] | | | | | | |
| 1411 | 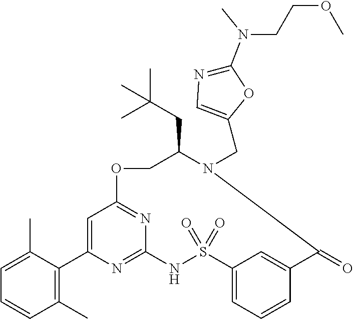 [SMILES: COCCN(C)c1ncc(CN2C(=O)c3cccc(c3)S(=O)(=O)Nc3nc(cc(-c4c(C)cccc4C)n3)OC[C@H]2CC(C)(C)C)o1] | | | | | | |
| 1303 | 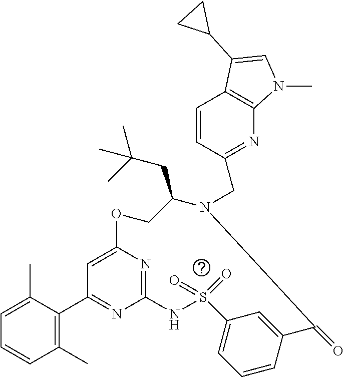 [SMILES: Cc1cccc(C)c1-c1cc2nc(n1)NS(=O)(=O)c1cccc(c1)C(=O)N(Cc1ccc3c(C4CC4)cn(C)c3n1)[C@H](CC(C)(C)C)CO2] | | | +++ | | | |
| 1412 | 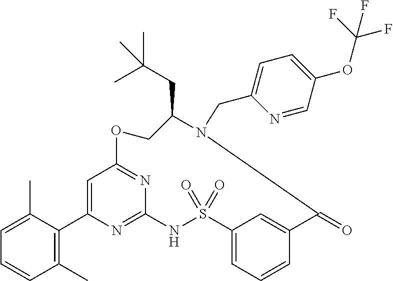 [SMILES: Cc1cccc(C)c1-c1cc2nc(n1)NS(=O)(=O)c1cccc(c1)C(=O)N(Cc1ccc(OC(F)(F)F)cn1)[C@H](CC(C)(C)C)CO2] | +++ | +++ | | | | |
| 1413 | 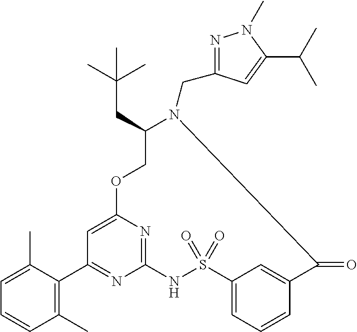 [SMILES: Cc1cccc(C)c1-c1cc2nc(n1)NS(=O)(=O)c1cccc(c1)C(=O)N(Cc1cc(C(C)C)n(C)n1)[C@H](CC(C)(C)C)CO2] | | | | | | ++ |
| 1414 | 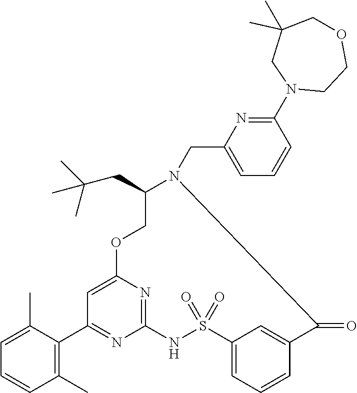 [SMILES: Cc1cccc(C)c1-c1cc2nc(n1)NS(=O)(=O)c1cccc(c1)C(=O)N(Cc1cccc(N3CCOCC(C)(C)C3)n1)[C@H](CC(C)(C)C)CO2] | +++ | +++ | | | | |
| 1415 | 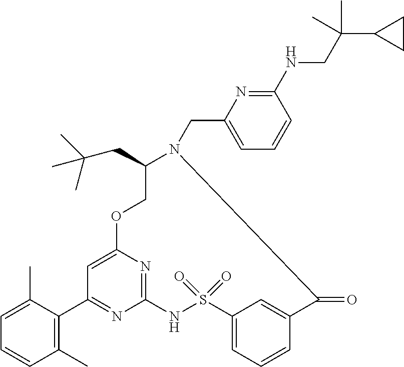 [SMILES: Cc1cccc(C)c1-c1cc2nc(n1)NS(=O)(=O)c1cccc(c1)C(=O)N(Cc1cccc(NCC(C)(C)C3CC3)n1)[C@H](CC(C)(C)C)CO2] | | | | | | |
| 1416 | 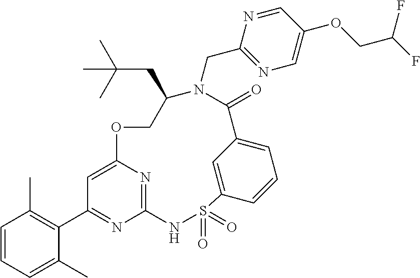 [SMILES: Cc1cccc(C)c1-c1cc2nc(n1)NS(=O)(=O)c1cccc(c1)C(=O)N(Cc1ncc(OCC(F)F)cn1)[C@H](CC(C)(C)C)CO2] | +++ | +++ | | | | |
| 1417 | 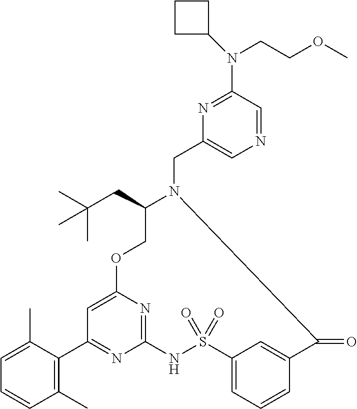 [SMILES: COCCN(c1cncc(CN2C(=O)c3cccc(c3)S(=O)(=O)Nc3nc(cc(-c4c(C)cccc4C)n3)OC[C@H]2CC(C)(C)C)n1)C1CCC1] | +++ | +++ | | | | |
| 1418 | 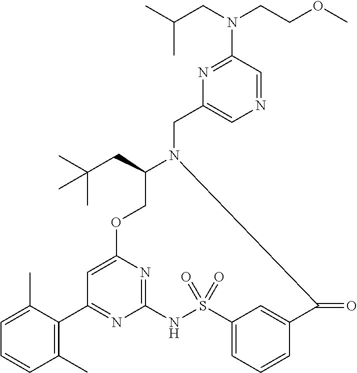 [SMILES: COCCN(CC(C)C)c1cncc(CN2C(=O)c3cccc(c3)S(=O)(=O)Nc3nc(cc(-c4c(C)cccc4C)n3)OC[C@H]2CC(C)(C)C)n1] | +++ | +++ | | | | |
| 1419 | 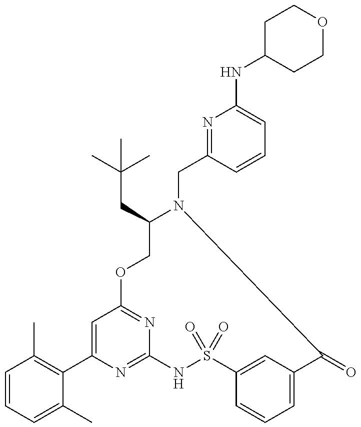 [SMILES: Cc1cccc(C)c1-c1cc2nc(n1)NS(=O)(=O)c1cccc(c1)C(=O)N(Cc1cccc(NC3CCOCC3)n1)[C@H](CC(C)(C)C)CO2] | +++ | +++ | | | | |
| 1420 | 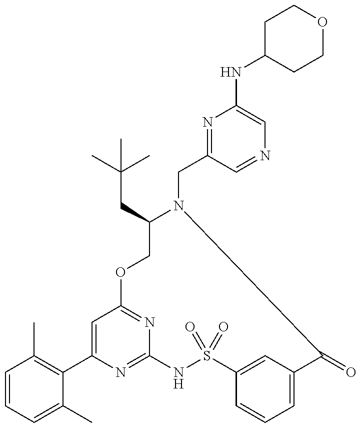 [SMILES: Cc1cccc(C)c1-c1cc2nc(n1)NS(=O)(=O)c1cccc(c1)C(=O)N(Cc1cncc(NC3CCOCC3)n1)[C@H](CC(C)(C)C)CO2] | +++ | +++ | | | | |
| 1305 | 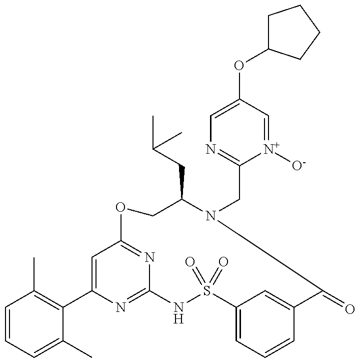 [SMILES: Cc1cccc(C)c1-c1cc2nc(n1)NS(=O)(=O)c1cccc(c1)C(=O)N(Cc1ncc(OC3CCCC3)c[n+]1[O-])[C@H](CC(C)C)CO2] | | | | | | +++ |
| 1421 | 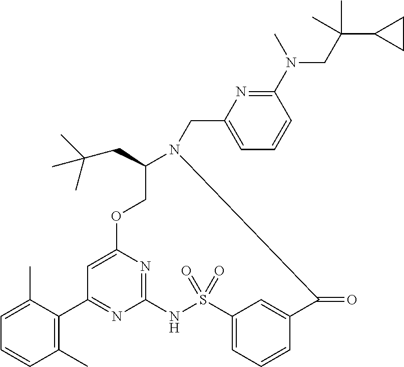 [SMILES: Cc1cccc(C)c1-c1cc2nc(n1)NS(=O)(=O)c1cccc(c1)C(=O)N(Cc1cccc(N(C)CC(C)(C)C3CC3)n1)[C@H](CC(C)(C)C)CO2] | | | +++ | | | |
| 1422 | 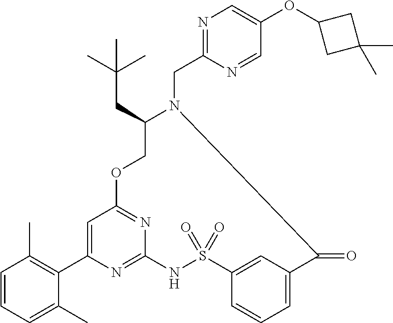 [SMILES: Cc1cccc(C)c1-c1cc2nc(n1)NS(=O)(=O)c1cccc(c1)C(=O)N(Cc1ncc(OC3CC(C)(C)C3)cn1)[C@H](CC(C)(C)C)CO2] | +++ | +++ | | | | |
| 1423 | 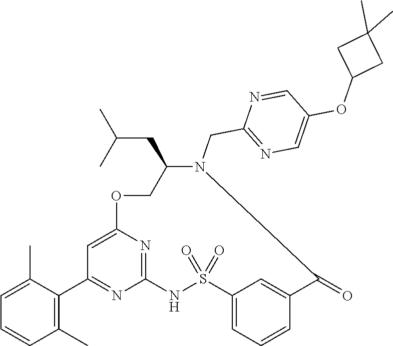 [SMILES: Cc1cccc(C)c1-c1cc2nc(n1)NS(=O)(=O)c1cccc(c1)C(=O)N(Cc1ncc(OC3CC(C)(C)C3)cn1)[C@H](CC(C)C)CO2] | ++ | +++ | | | | |
| 1424 | 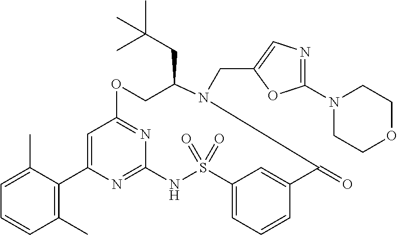 [SMILES: Cc1cccc(C)c1-c1cc2nc(n1)NS(=O)(=O)c1cccc(c1)C(=O)N(Cc1cnc(N3CCOCC3)o1)[C@H](CC(C)(C)C)CO2] | | | | | | |
| 1425 | 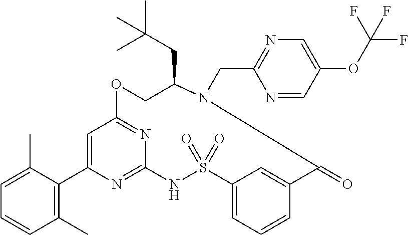 [SMILES: Cc1cccc(C)c1-c1cc2nc(n1)NS(=O)(=O)c1cccc(c1)C(=O)N(Cc1ncc(OC(F)(F)F)cn1)[C@H](CC(C)(C)C)CO2] | +++ | +++ | | | | |
| 1297 | 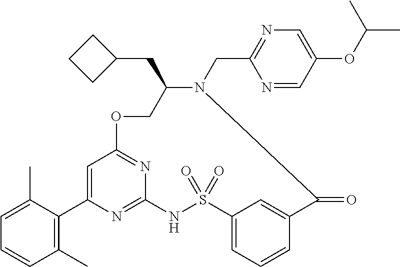 [SMILES: Cc1cccc(C)c1-c1cc2nc(n1)NS(=O)(=O)c1cccc(c1)C(=O)N(Cc1ncc(OC(C)C)cn1)[C@H](CC1CCC1)CO2] | +++ | +++ | | +++ | +++ | +++ |
| 1426 | 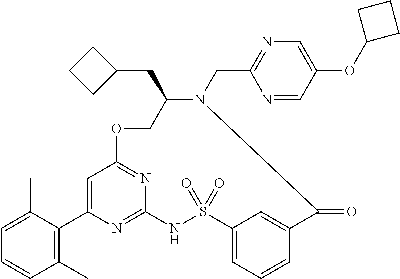 [SMILES: Cc1cccc(C)c1-c1cc2nc(n1)NS(=O)(=O)c1cccc(c1)C(=O)N(Cc1ncc(OC3CCC3)cn1)[C@H](CC1CCC1)CO2] | +++ | +++ | | | | |
| 1427 | 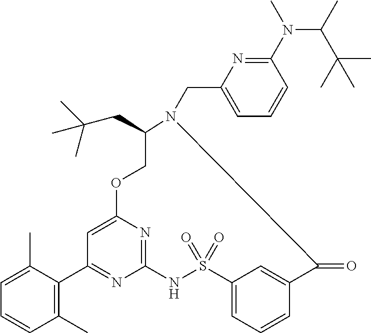 [SMILES: Cc1cccc(C)c1-c1cc2nc(n1)NS(=O)(=O)c1cccc(c1)C(=O)N(Cc1cccc(N(C)C(C)C(C)(C)C)n1)[C@H](CC(C)(C)C)CO2] | | | +++ | | | |
| 1428 | 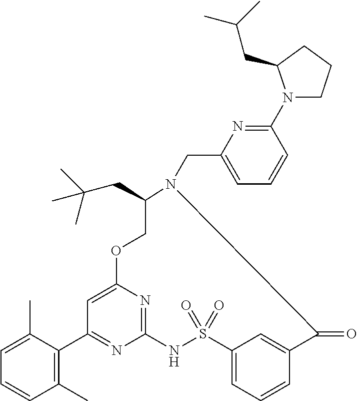 [SMILES: Cc1cccc(C)c1-c1cc2nc(n1)NS(=O)(=O)c1cccc(c1)C(=O)N(Cc1cccc(N3CCC[C@@H]3CC(C)C)n1)[C@H](CC(C)(C)C)CO2] | +++ | +++ | | | | |
| 1429 | 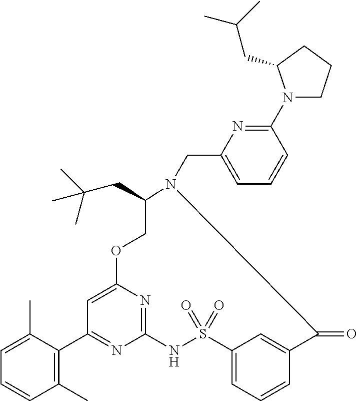 [SMILES: Cc1cccc(C)c1-c1cc2nc(n1)NS(=O)(=O)c1cccc(c1)C(=O)N(Cc1cccc(N3CCC[C@H]3CC(C)C)n1)[C@H](CC(C)(C)C)CO2] | +++ | +++ | | | | |
| 1430 | 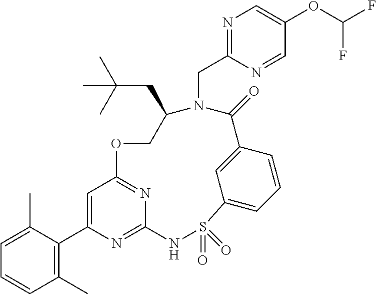 [SMILES: Cc1cccc(C)c1-c1cc2nc(n1)NS(=O)(=O)c1cccc(c1)C(=O)N(Cc1ncc(OC(F)F)cn1)[C@H](CC(C)(C)C)CO2] | +++ | +++ | | | | |
| 1431 | 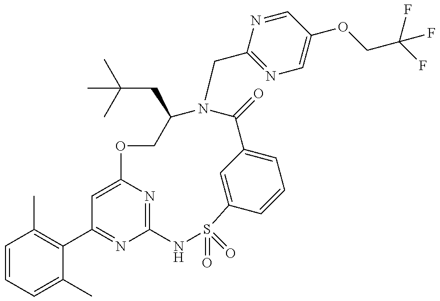 [SMILES: Cc1cccc(C)c1-c1cc2nc(n1)NS(=O)(=O)c1cccc(c1)C(=O)N(Cc1ncc(OCC(F)(F)F)cn1)[C@H](CC(C)(C)C)CO2] | +++ | +++ | | | | |
| 1432 | 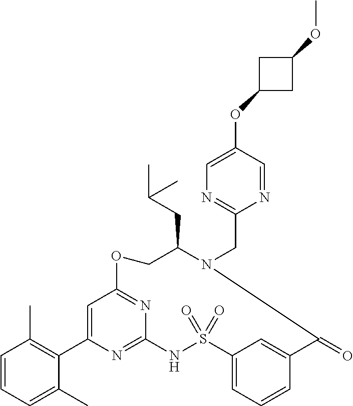 [SMILES: CO[C@H]1C[C@@H](Oc2cnc(CN3C(=O)c4cccc(c4)S(=O)(=O)Nc4nc(cc(-c5c(C)cccc5C)n4)OC[C@H]3CC(C)C)nc2)C1] | +++ | +++ | | | | |
| 1433 | 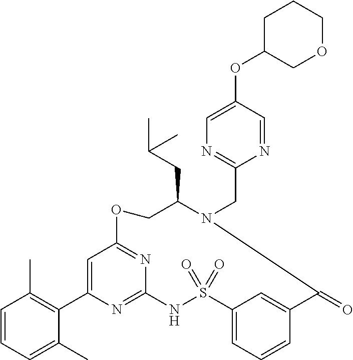 [SMILES: Cc1cccc(C)c1-c1cc2nc(n1)NS(=O)(=O)c1cccc(c1)C(=O)N(Cc1ncc(OC3CCCOC3)cn1)[C@H](CC(C)C)CO2] | | | +++ | | | |
| 1434 | 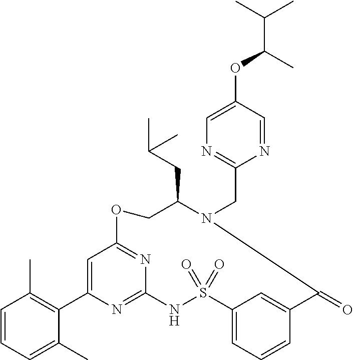 [SMILES: Cc1cccc(C)c1-c1cc2nc(n1)NS(=O)(=O)c1cccc(c1)C(=O)N(Cc1ncc(O[C@H](C)C(C)C)cn1)[C@H](CC(C)C)CO2] | ++ | +++ | | | | |
| 1435 | 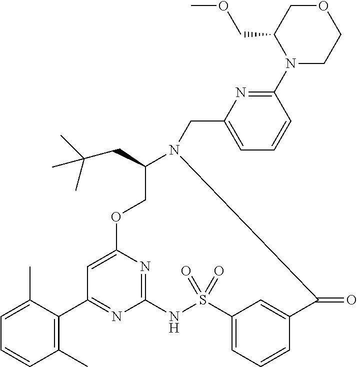 [SMILES: COC[C@@H]1COCCN1c1cccc(CN2C(=O)c3cccc(c3)S(=O)(=O)Nc3nc(cc(-c4c(C)cccc4C)n3)OC[C@H]2CC(C)(C)C)n1] | | | +++ | | | |
| 1436 | 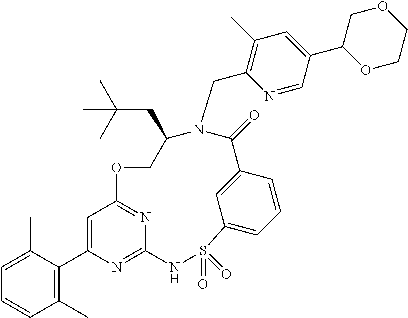 [SMILES: Cc1cc(C2COCCO2)cnc1CN1C(=O)c2cccc(c2)S(=O)(=O)Nc2nc(cc(-c3c(C)cccc3C)n2)OC[C@H]1CC(C)(C)C] | +++ | +++ | +++ | | | |
| 1437 | 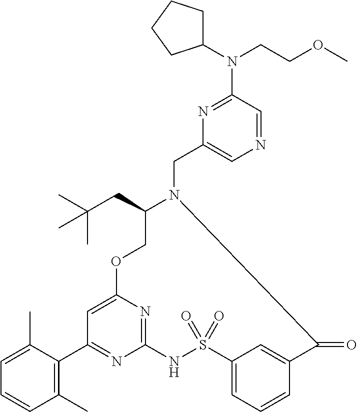 [SMILES: COCCN(c1cncc(CN2C(=O)c3cccc(c3)S(=O)(=O)Nc3nc(cc(-c4c(C)cccc4C)n3)OC[C@H]2CC(C)(C)C)n1)C1CCCC1] | +++ | +++ | | | | |
| 1438 | 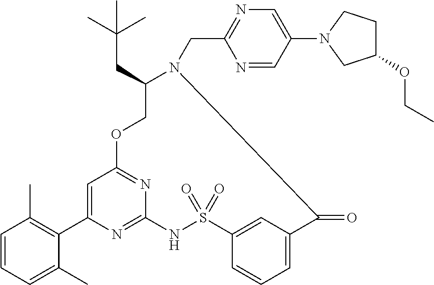 [SMILES: CCO[C@H]1CCN(c2cnc(CN3C(=O)c4cccc(c4)S(=O)(=O)Nc4nc(cc(-c5c(C)cccc5C)n4)OC[C@H]3CC(C)(C)C)nc2)C1] | +++ | +++ | | | | |
| 1439 | 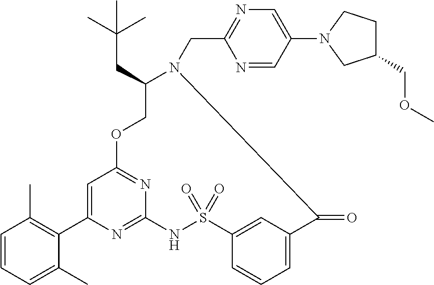 [SMILES: COC[C@H]1CCN(c2cnc(CN3C(=O)c4cccc(c4)S(=O)(=O)Nc4nc(cc(-c5c(C)cccc5C)n4)OC[C@H]3CC(C)(C)C)nc2)C1] | +++ | +++ | | | | |
| 1440 | 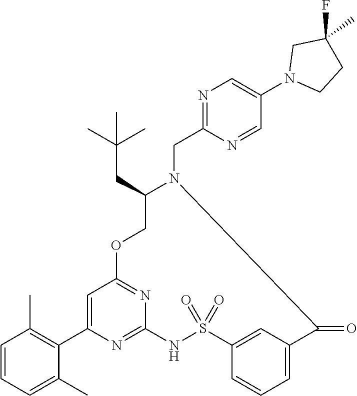 [SMILES: Cc1cccc(C)c1-c1cc2nc(n1)NS(=O)(=O)c1cccc(c1)C(=O)N(Cc1ncc(N3CC[C@](C)(F)C3)cn1)[C@H](CC(C)(C)C)CO2] | +++ | +++ | | | | |
| 1441 | 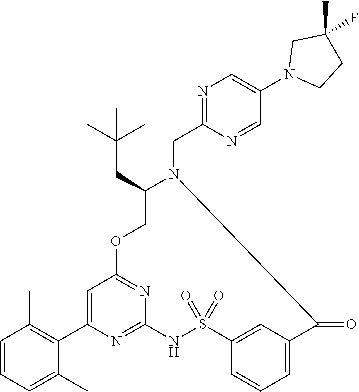 [SMILES: Cc1cccc(C)c1-c1cc2nc(n1)NS(=O)(=O)c1cccc(c1)C(=O)N(Cc1ncc(N3CC[C@@](C)(F)C3)cn1)[C@H](CC(C)(C)C)CO2] | +++ | +++ | | | | |
| 1442 | 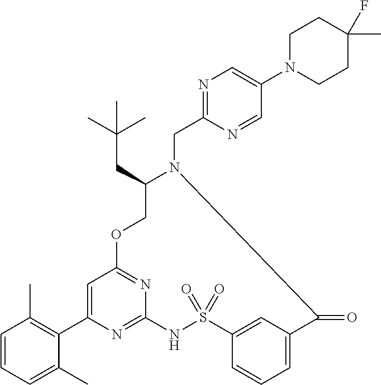 [SMILES: Cc1cccc(C)c1-c1cc2nc(n1)NS(=O)(=O)c1cccc(c1)C(=O)N(Cc1ncc(N3CCC(C)(F)CC3)cn1)[C@H](CC(C)(C)C)CO2] | | | +++ | | | |
| 1443 | 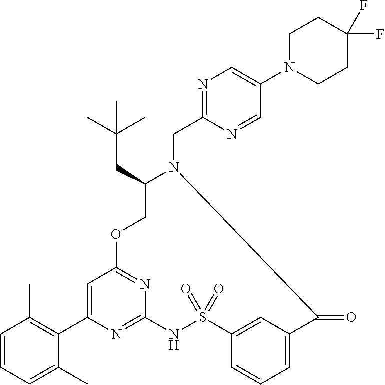 [SMILES: Cc1cccc(C)c1-c1cc2nc(n1)NS(=O)(=O)c1cccc(c1)C(=O)N(Cc1ncc(N3CCC(F)(F)CC3)cn1)[C@H](CC(C)(C)C)CO2] | | | +++ | | | |
| 1444 | 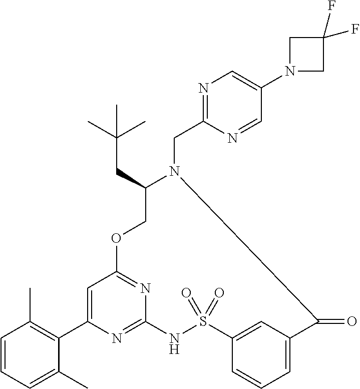 [SMILES: Cc1cccc(C)c1-c1cc2nc(n1)NS(=O)(=O)c1cccc(c1)C(=O)N(Cc1ncc(N3CC(F)(F)C3)cn1)[C@H](CC(C)(C)C)CO2] | +++ | +++ | | | | |
| 1445 | 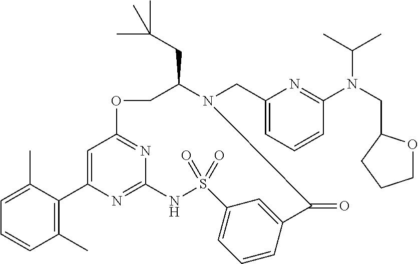 [SMILES: Cc1cccc(C)c1-c1cc2nc(n1)NS(=O)(=O)c1cccc(c1)C(=O)N(Cc1cccc(N(CC3CCCO3)C(C)C)n1)[C@H](CC(C)(C)C)CO2] | ++ | +++ | | | | |
| 1446 | 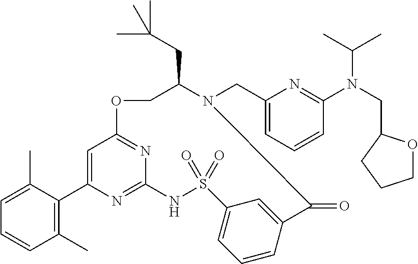 [SMILES: Cc1cccc(C)c1-c1cc2nc(n1)NS(=O)(=O)c1cccc(c1)C(=O)N(Cc1cccc(N(CC3CCCO3)C(C)C)n1)[C@H](CC(C)(C)C)CO2] | +++ | +++ | | | | |
| 1447 | 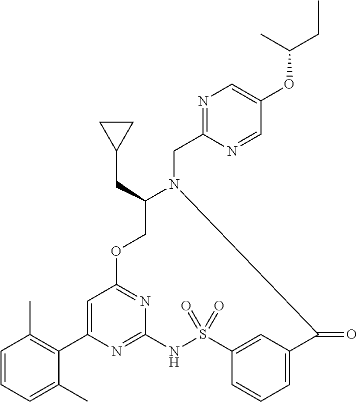 [SMILES: CC[C@@H](C)Oc1cnc(CN2C(=O)c3cccc(c3)S(=O)(=O)Nc3nc(cc(-c4c(C)cccc4C)n3)OC[C@H]2CC2CC2)nc1] | +++ | +++ | | | | |
| 1448 | 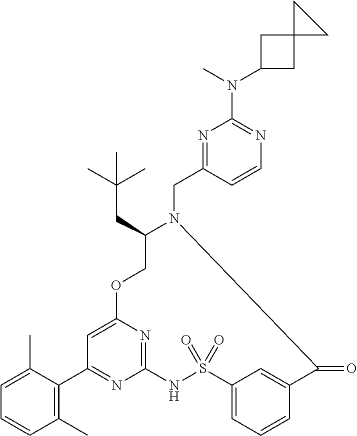 [SMILES: Cc1cccc(C)c1-c1cc2nc(n1)NS(=O)(=O)c1cccc(c1)C(=O)N(Cc1ccnc(N(C)C3CC4(CC4)C3)n1)[C@H](CC(C)(C)C)CO2] | | | +++ | +++ | +++ | |
| 1449 | 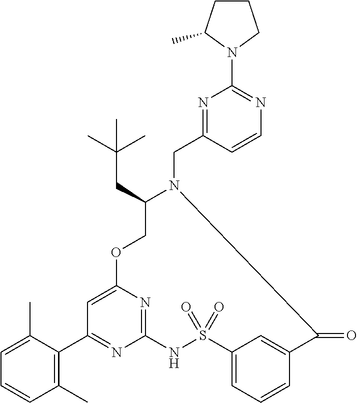 [SMILES: Cc1cccc(C)c1-c1cc2nc(n1)NS(=O)(=O)c1cccc(c1)C(=O)N(Cc1ccnc(N3CCC[C@H]3C)n1)[C@H](CC(C)(C)C)CO2] | +++ | +++ | | | | |
| 1450 | 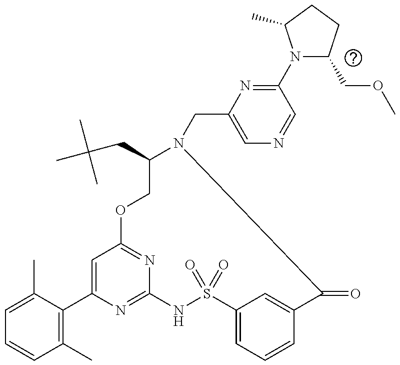 [SMILES: COC[C@H]1CC[C@@H](C)N1c1cncc(CN2C(=O)c3cccc(c3)S(=O)(=O)Nc3nc(cc(-c4c(C)cccc4C)n3)OC[C@H]2CC(C)(C)C)n1] | +++ | +++ | | | | |
| 1451 | 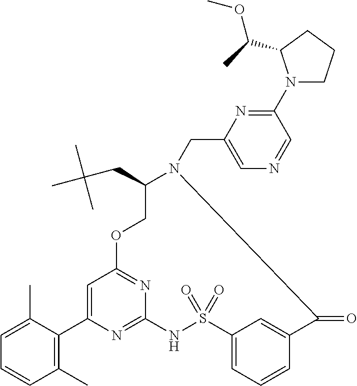 [SMILES: CO[C@H](C)[C@@H]1CCCN1c1cncc(CN2C(=O)c3cccc(c3)S(=O)(=O)Nc3nc(cc(-c4c(C)cccc4C)n3)OC[C@H]2CC(C)(C)C)n1] | +++ | +++ | | | | |
| 1452 | 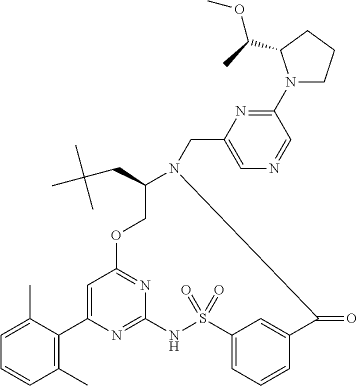 [SMILES: CO[C@H](C)[C@@H]1CCCN1c1cncc(CN2C(=O)c3cccc(c3)S(=O)(=O)Nc3nc(cc(-c4c(C)cccc4C)n3)OC[C@H]2CC(C)(C)C)n1] | +++ | +++ | | | | |
| 1453 | 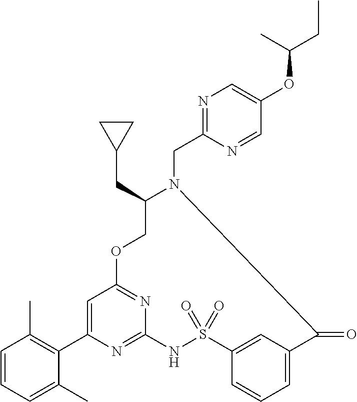 [SMILES: CC[C@H](C)Oc1cnc(CN2C(=O)c3cccc(c3)S(=O)(=O)Nc3nc(cc(-c4c(C)cccc4C)n3)OC[C@H]2CC2CC2)nc1] | ++ | +++ | | | | |
| 1454 | 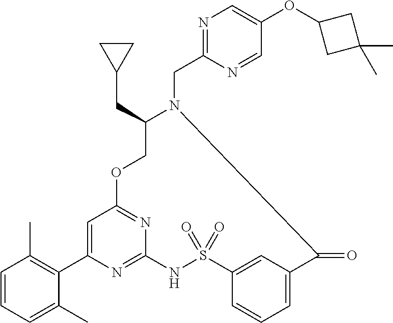 [SMILES: Cc1cccc(C)c1-c1cc2nc(n1)NS(=O)(=O)c1cccc(c1)C(=O)N(Cc1ncc(OC3CC(C)(C)C3)cn1)[C@H](CC1CC1)CO2] | +++ | +++ | | | | |
| 1455 | 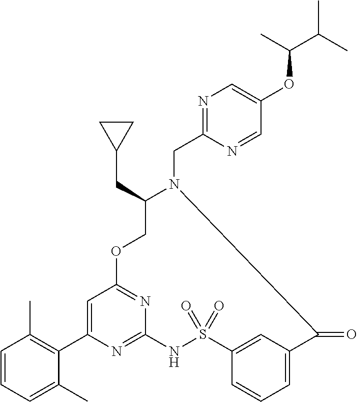 [SMILES: Cc1cccc(C)c1-c1cc2nc(n1)NS(=O)(=O)c1cccc(c1)C(=O)N(Cc1ncc(O[C@@H](C)C(C)C)cn1)[C@H](CC1CC1)CO2] | +++ | +++ | | | | |
| 1456 | 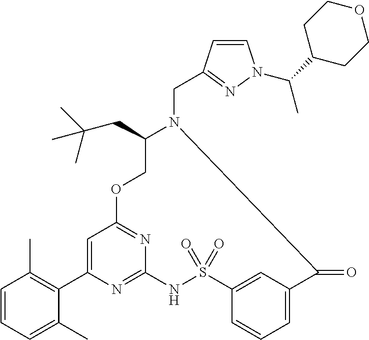 [SMILES: Cc1cccc(C)c1-c1cc2nc(n1)NS(=O)(=O)c1cccc(c1)C(=O)N(Cc1ccn([C@@H](C)C3CCOCC3)n1)[C@H](CC(C)(C)C)CO2] | | | | | | +++ |
| 1457 | 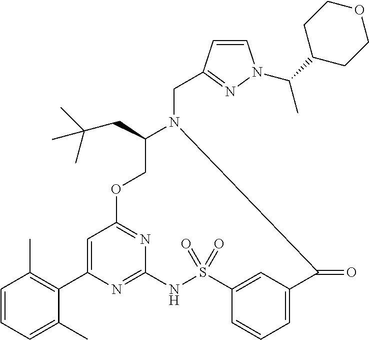 [SMILES: Cc1cccc(C)c1-c1cc2nc(n1)NS(=O)(=O)c1cccc(c1)C(=O)N(Cc1ccn([C@@H](C)C3CCOCC3)n1)[C@H](CC(C)(C)C)CO2] | | | | | | +++ |
| 1458 | 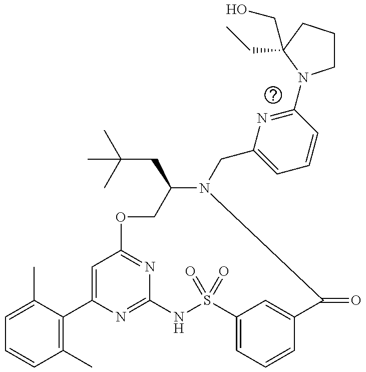 [SMILES: CC[C@]1(CO)CCCN1c1cccc(CN2C(=O)c3cccc(c3)S(=O)(=O)Nc3nc(cc(-c4c(C)cccc4C)n3)OC[C@H]2CC(C)(C)C)n1] | | | +++ | | | |
| 1459 | 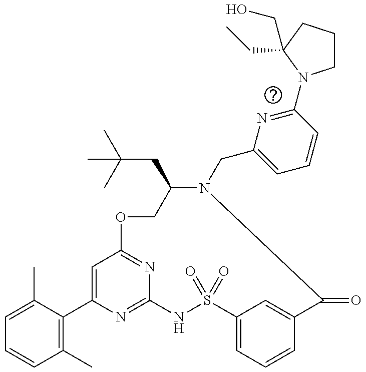 [SMILES: CC[C@]1(CO)CCCN1c1cccc(CN2C(=O)c3cccc(c3)S(=O)(=O)Nc3nc(cc(-c4c(C)cccc4C)n3)OC[C@H]2CC(C)(C)C)n1] | | | +++ | | | |
| 1460 | 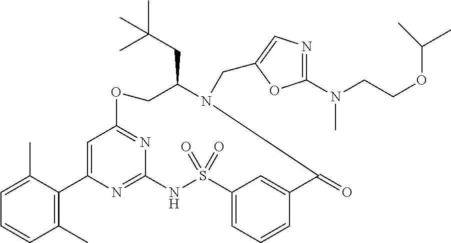 [SMILES: Cc1cccc(C)c1-c1cc2nc(n1)NS(=O)(=O)c1cccc(c1)C(=O)N(Cc1cnc(N(C)CCOC(C)C)o1)[C@H](CC(C)(C)C)CO2] | | | +++ | | | |
| 1304 | 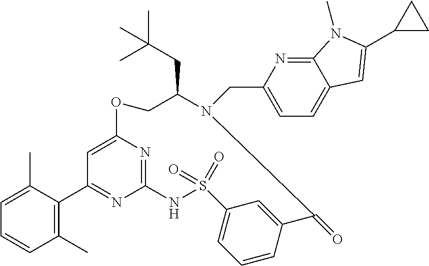 [SMILES: Cc1cccc(C)c1-c1cc2nc(n1)NS(=O)(=O)c1cccc(c1)C(=O)N(Cc1ccc3cc(C4CC4)n(C)c3n1)[C@H](CC(C)(C)C)CO2] | | | +++ | | | +++ |
| 1461 | 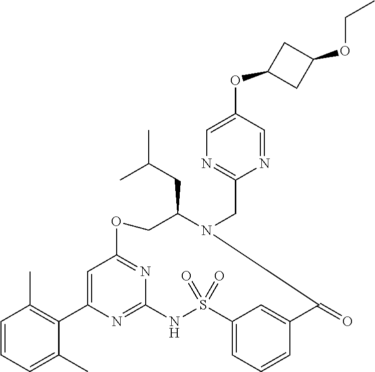 [SMILES: CCO[C@H]1C[C@@H](Oc2cnc(CN3C(=O)c4cccc(c4)S(=O)(=O)Nc4nc(cc(-c5c(C)cccc5C)n4)OC[C@H]3CC(C)C)nc2)C1] | +++ | +++ | | | | |
| 1462 | 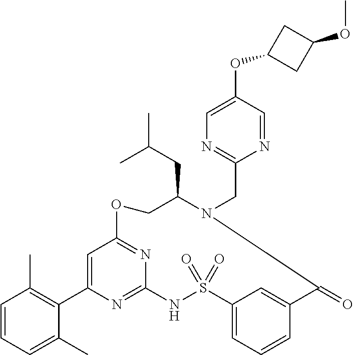 [SMILES: CO[C@H]1C[C@H](Oc2cnc(CN3C(=O)c4cccc(c4)S(=O)(=O)Nc4nc(cc(-c5c(C)cccc5C)n4)OC[C@H]3CC(C)C)nc2)C1] | +++ | +++ | | | | |
| 1463 | 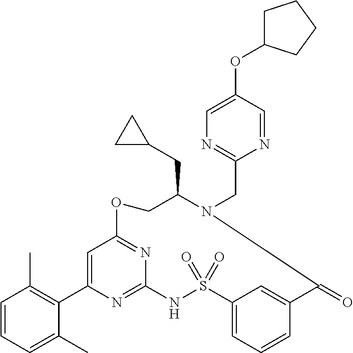 [SMILES: Cc1cccc(C)c1-c1cc2nc(n1)NS(=O)(=O)c1cccc(c1)C(=O)N(Cc1ncc(OC3CCCC3)cn1)[C@H](CC1CC1)CO2] | +++ | +++ | | | | |
| 1464 | 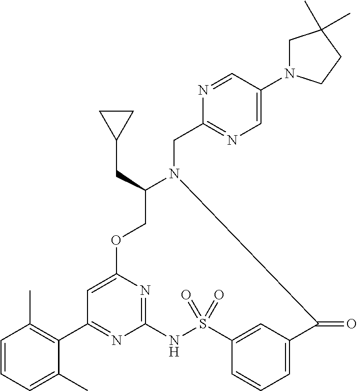 [SMILES: Cc1cccc(C)c1-c1cc2nc(n1)NS(=O)(=O)c1cccc(c1)C(=O)N(Cc1ncc(N3CCC(C)(C)C3)cn1)[C@H](CC1CC1)CO2] | +++ | +++ | | | | |
| 1465 | 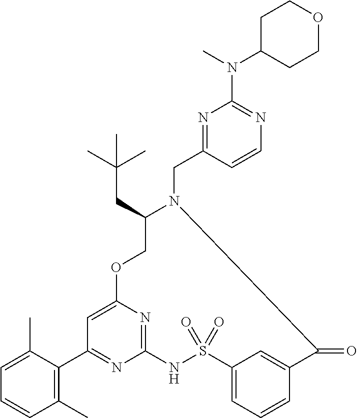 [SMILES: Cc1cccc(C)c1-c1cc2nc(n1)NS(=O)(=O)c1cccc(c1)C(=O)N(Cc1ccnc(N(C)C3CCOCC3)n1)[C@H](CC(C)(C)C)CO2] | | | | | | |
| 1466 | 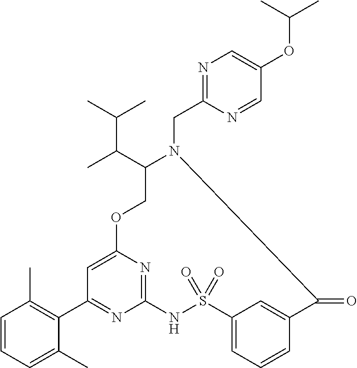 [SMILES: Cc1cccc(C)c1-c1cc2nc(n1)NS(=O)(=O)c1cccc(c1)C(=O)N(Cc1ncc(OC(C)C)cn1)C(C(C)C(C)C)CO2] | | | +++ | | | |
| 1467 | 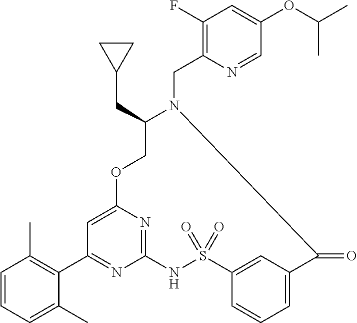 [SMILES: Cc1cccc(C)c1-c1cc2nc(n1)NS(=O)(=O)c1cccc(c1)C(=O)N(Cc1ncc(OC(C)C)cc1F)[C@H](CC1CC1)CO2] | ++ | +++ | | | | |
| 1468 | 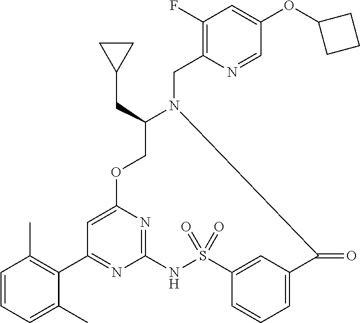 [SMILES: Cc1cccc(C)c1-c1cc2nc(n1)NS(=O)(=O)c1cccc(c1)C(=O)N(Cc1ncc(OC3CCC3)cc1F)[C@H](CC1CC1)CO2] | +++ | +++ | | | | |
| 1469 | 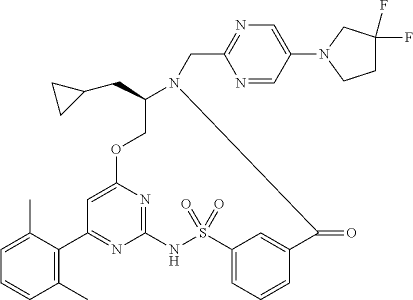 [SMILES: Cc1cccc(C)c1-c1cc2nc(n1)NS(=O)(=O)c1cccc(c1)C(=O)N(Cc1ncc(N3CCC(F)(F)C3)cn1)[C@H](CC1CC1)CO2] | +++ | +++ | | | | |
| 1470 | 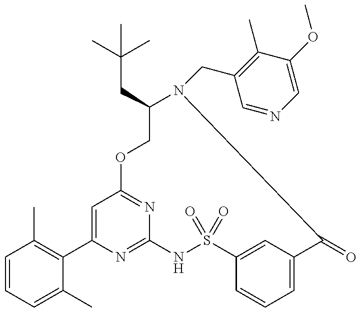 [SMILES: COc1cncc(CN2C(=O)c3cccc(c3)S(=O)(=O)Nc3nc(cc(-c4c(C)cccc4C)n3)OC[C@H]2CC(C)(C)C)c1C] | | | | | | |
| 1471 | 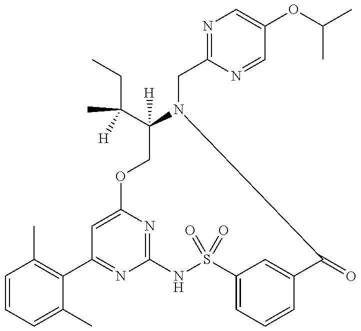 [SMILES: CC[C@H](C)[C@@H]1COc2cc(-c3c(C)cccc3C)nc(n2)NS(=O)(=O)c2cccc(c2)C(=O)N1Cc1ncc(OC(C)C)cn1] | +++ | +++ | | | | |
| 1472 | 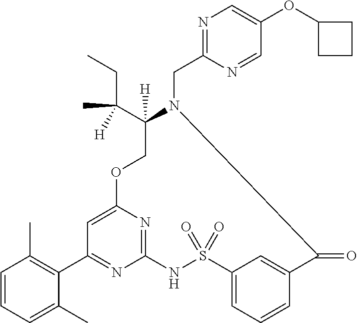 [SMILES: CC[C@H](C)[C@@H]1COc2cc(-c3c(C)cccc3C)nc(n2)NS(=O)(=O)c2cccc(c2)C(=O)N1Cc1ncc(OC2CCC2)cn1] | +++ | +++ | +++ | | | |
| 1473 | 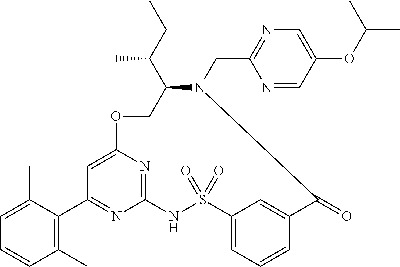 [SMILES: CC[C@@H](C)[C@@H]1COc2cc(-c3c(C)cccc3C)nc(n2)NS(=O)(=O)c2cccc(c2)C(=O)N1Cc1ncc(OC(C)C)cn1] | +++ | +++ | | | | |
| 1474 | 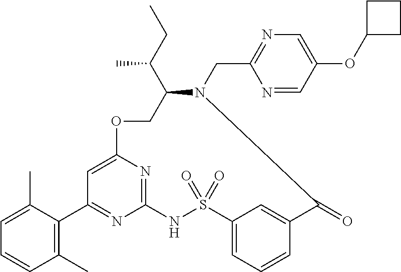 [SMILES: CC[C@@H](C)[C@@H]1COc2cc(-c3c(C)cccc3C)nc(n2)NS(=O)(=O)c2cccc(c2)C(=O)N1Cc1ncc(OC2CCC2)cn1] | +++ | +++ | | | | |
| 1475 | 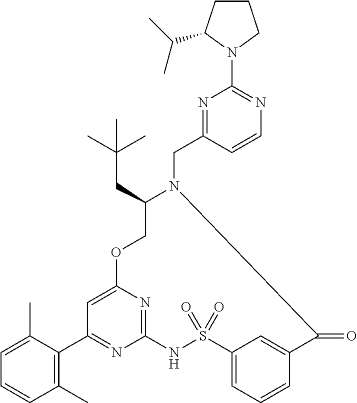 [SMILES: Cc1cccc(C)c1-c1cc2nc(n1)NS(=O)(=O)c1cccc(c1)C(=O)N(Cc1ccnc(N3CCC[C@H]3C(C)C)n1)[C@H](CC(C)(C)C)CO2] | +++ | +++ | | | | |
| 1476 | 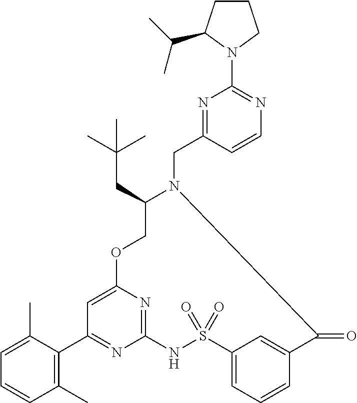 [SMILES: Cc1cccc(C)c1-c1cc2nc(n1)NS(=O)(=O)c1cccc(c1)C(=O)N(Cc1ccnc(N3CCC[C@@H]3C(C)C)n1)[C@H](CC(C)(C)C)CO2] | +++ | +++ | | | | |
| 1477 | 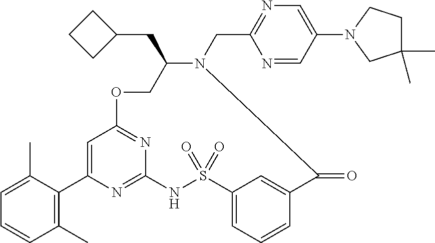 [SMILES: Cc1cccc(C)c1-c1cc2nc(n1)NS(=O)(=O)c1cccc(c1)C(=O)N(Cc1ncc(N3CCC(C)(C)C3)cn1)[C@H](CC1CCC1)CO2] | +++ | +++ | | | | |
| 1478 | 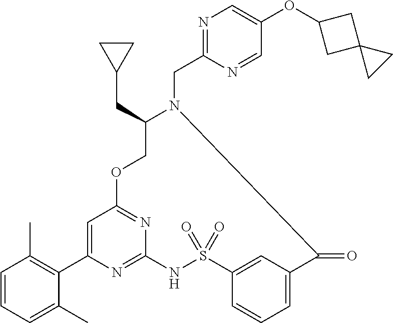 [SMILES: Cc1cccc(C)c1-c1cc2nc(n1)NS(=O)(=O)c1cccc(c1)C(=O)N(Cc1ncc(OC3CC4(CC4)C3)cn1)[C@H](CC1CC1)CO2] | +++ | +++ | | | | |
| 1479 | 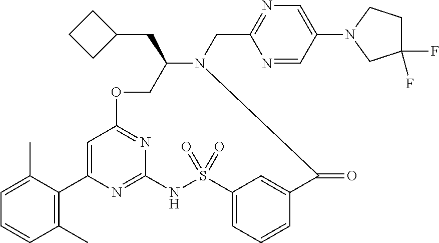 [SMILES: Cc1cccc(C)c1-c1cc2nc(n1)NS(=O)(=O)c1cccc(c1)C(=O)N(Cc1ncc(N3CCC(F)(F)C3)cn1)[C@H](CC1CCC1)CO2] | +++ | +++ | | | | |
| 1480 | 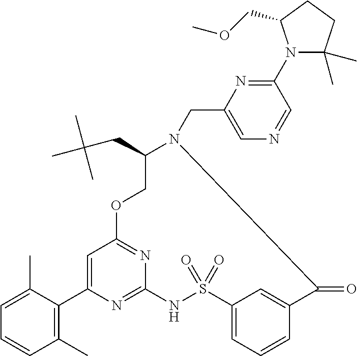 [SMILES: COC[C@@H]1CCC(C)(C)N1c1cncc(CN2C(=O)c3cccc(c3)S(=O)(=O)Nc3nc(cc(-c4c(C)cccc4C)n3)OC[C@H]2CC(C)(C)C)n1] | +++ | +++ | | | | |
| 1481 | 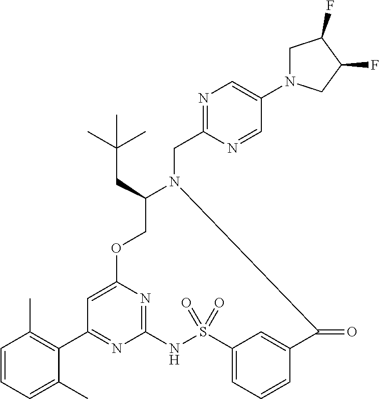 [SMILES: Cc1cccc(C)c1-c1cc2nc(n1)NS(=O)(=O)c1cccc(c1)C(=O)N(Cc1ncc(N3C[C@@H](F)[C@@H](F)C3)cn1)[C@H](CC(C)(C)C)CO2] | +++ | +++ | | | | |
| 1482 | 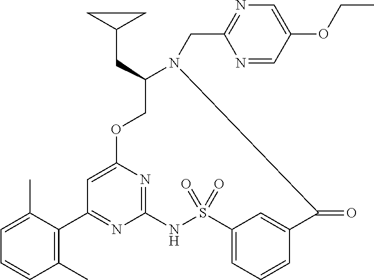 [SMILES: CCOc1cnc(CN2C(=O)c3cccc(c3)S(=O)(=O)Nc3nc(cc(-c4c(C)cccc4C)n3)OC[C@H]2CC2CC2)nc1] | +++ | +++ | | | | |
| 1483 | 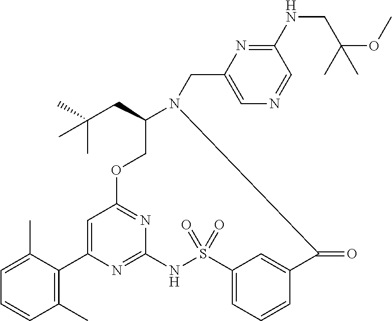 | +++ | +++ | | | | |
| 1484 | 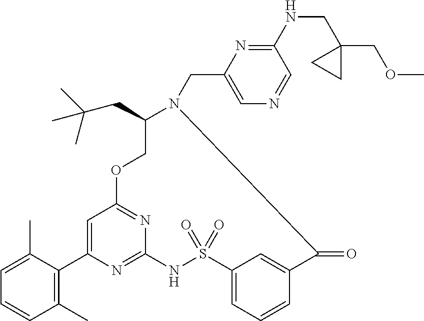 [SMILES: COCC1(CNc2cncc(CN3C(=O)c4cccc(c4)S(=O)(=O)Nc4nc(cc(-c5c(C)cccc5C)n4)OC[C@H]3CC(C)(C)C)n2)CC1] | | | +++ | | | |
| 1485 | 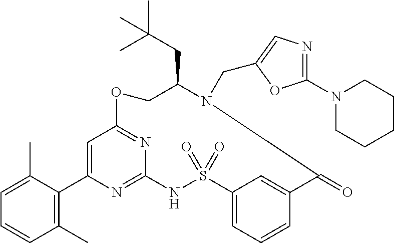 [SMILES: Cc1cccc(C)c1-c1cc2nc(n1)NS(=O)(=O)c1cccc(c1)C(=O)N(Cc1cnc(N3CCCCC3)o1)[C@H](CC(C)(C)C)CO2] | | | | | | |
| 1296 | 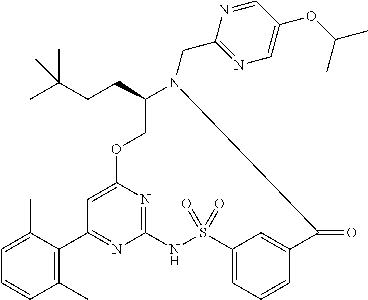 [SMILES: Cc1cccc(C)c1-c1cc2nc(n1)NS(=O)(=O)c1cccc(c1)C(=O)N(Cc1ncc(OC(C)C)cn1)[C@H](CCC(C)(C)C)CO2] | +++ | +++ | | | | +++ |
| 1486 | 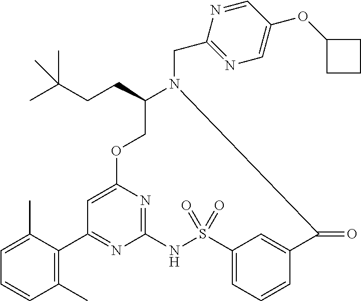 [SMILES: Cc1cccc(C)c1-c1cc2nc(n1)NS(=O)(=O)c1cccc(c1)C(=O)N(Cc1ncc(OC3CCC3)cn1)[C@H](CCC(C)(C)C)CO2] | | | +++ | | | |
| 1487 | 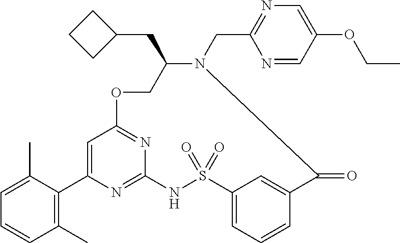 [SMILES: CCOc1cnc(CN2C(=O)c3cccc(c3)S(=O)(=O)Nc3nc(cc(-c4c(C)cccc4C)n3)OC[C@H]2CC2CCC2)nc1] | +++ | +++ | | | | |
| 1488 | 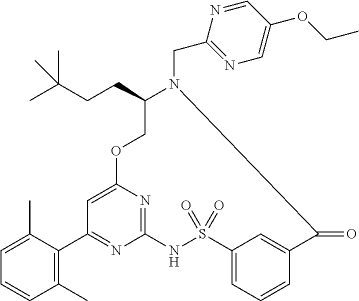 [SMILES: CCOc1cnc(CN2C(=O)c3cccc(c3)S(=O)(=O)Nc3nc(cc(-c4c(C)cccc4C)n3)OC[C@H]2CCC(C)(C)C)nc1] | +++ | +++ | | | | |
| 1489 | 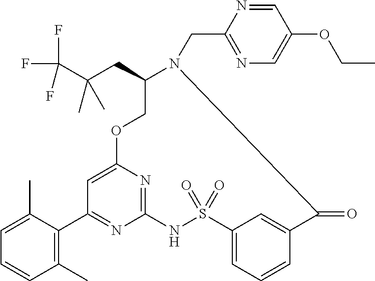 [SMILES: CCOc1cnc(CN2C(=O)c3cccc(c3)S(=O)(=O)Nc3nc(cc(-c4c(C)cccc4C)n3)OC[C@H]2CC(C)(C)C(F)(F)F)nc1] | +++ | +++ | | | | |
| 1490 | 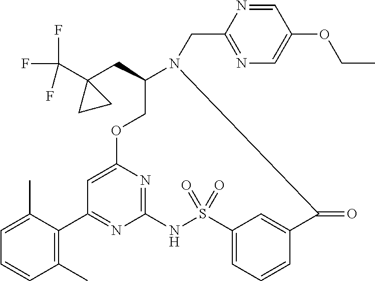 [SMILES: CCOc1cnc(CN2C(=O)c3cccc(c3)S(=O)(=O)Nc3nc(cc(-c4c(C)cccc4C)n3)OC[C@H]2CC2(C(F)(F)F)CC2)nc1] | +++ | +++ | | | | |
| 1491 | 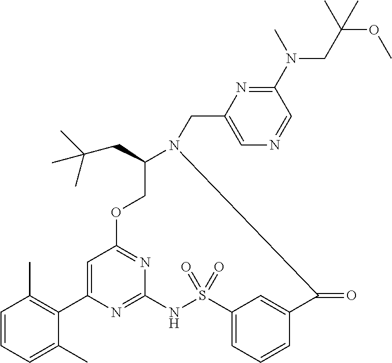 [SMILES: COC(C)(C)CN(C)c1cncc(CN2C(=O)c3cccc(c3)S(=O)(=O)Nc3nc(cc(-c4c(C)cccc4C)n3)OC[C@H]2CC(C)(C)C)n1] | +++ | +++ | | | | |
| 1492 | 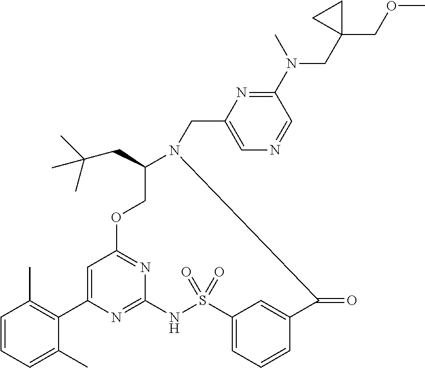 [SMILES: COCC1(CN(C)c2cncc(CN3C(=O)c4cccc(c4)S(=O)(=O)Nc4nc(cc(-c5c(C)cccc5C)n4)OC[C@H]3CC(C)(C)C)n2)CC1] | +++ | +++ | +++ | | | +++ |
| 1493 | 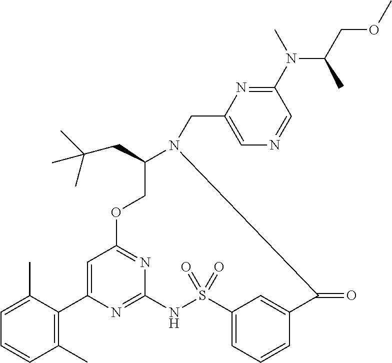 [SMILES: COC[C@@H](C)N(C)c1cncc(CN2C(=O)c3cccc(c3)S(=O)(=O)Nc3nc(cc(-c4c(C)cccc4C)n3)OC[C@H]2CC(C)(C)C)n1] | +++ | +++ | | | | +++ |
| 1494 | 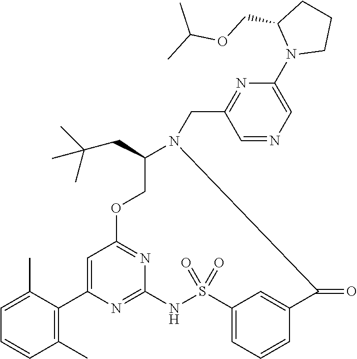 [SMILES: Cc1cccc(C)c1-c1cc2nc(n1)NS(=O)(=O)c1cccc(c1)C(=O)N(Cc1cncc(N3CCC[C@H]3COC(C)C)n1)[C@H](CC(C)(C)C)CO2] | +++ | +++ | | | | |
| 1495 | 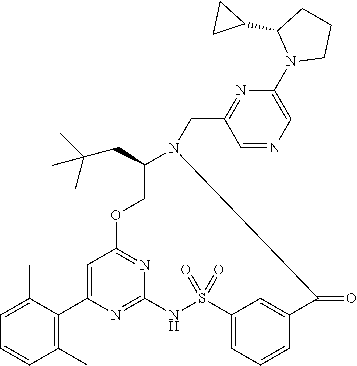 [SMILES: Cc1cccc(C)c1-c1cc2nc(n1)NS(=O)(=O)c1cccc(c1)C(=O)N(Cc1cncc(N3CCC[C@H]3C3CC3)n1)[C@H](CC(C)(C)C)CO2] | +++ | +++ | | | | +++ |
| 1496 | 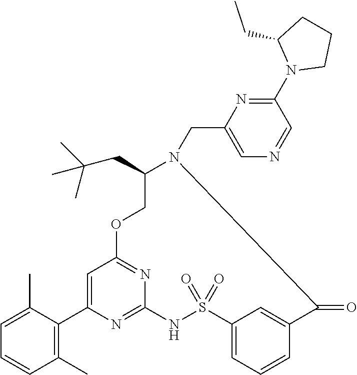 [SMILES: CC[C@@H]1CCCN1c1cncc(CN2C(=O)c3cccc(c3)S(=O)(=O)Nc3nc(cc(-c4c(C)cccc4C)n3)OC[C@H]2CC(C)(C)C)n1] | +++ | +++ | | | | +++ |
| 1497 | 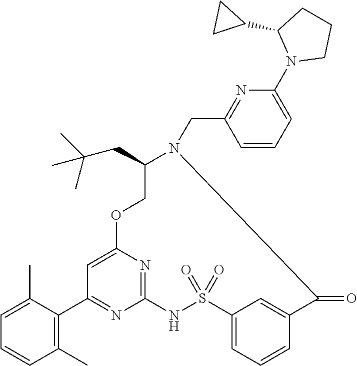 [SMILES: Cc1cccc(C)c1-c1cc2nc(n1)NS(=O)(=O)c1cccc(c1)C(=O)N(Cc1cccc(N3CCC[C@H]3C3CC3)n1)[C@H](CC(C)(C)C)CO2] | +++ | +++ | | | | |
| 1498 | 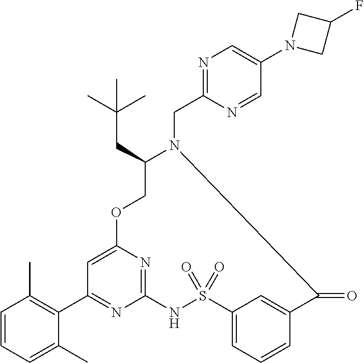 [SMILES: Cc1cccc(C)c1-c1cc2nc(n1)NS(=O)(=O)c1cccc(c1)C(=O)N(Cc1ncc(N3CC(F)C3)cn1)[C@H](CC(C)(C)C)CO2] | | | +++ | | | |
| 1499 | 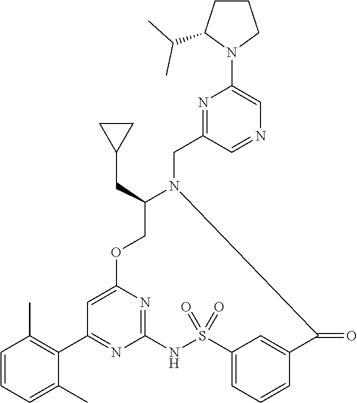 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](CC1CC1)CO2] | +++ | +++ | +++ | | | |
| 1500 | 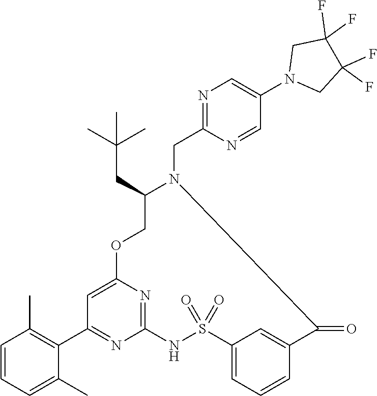 [SMILES: Cc1cccc(C)c1-c1cc2nc(n1)NS(=O)(=O)c1cccc(c1)C(=O)N(Cc1ncc(N3CC(F)(F)C(F)(F)C3)cn1)[C@H](CC(C)(C)C)CO2] | | | +++ | | | |
| 1501 | 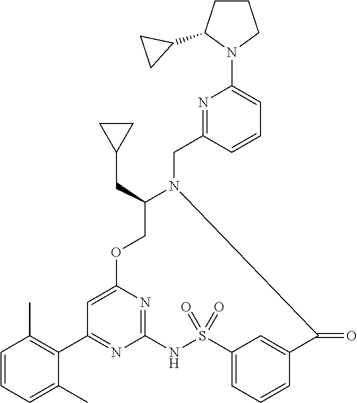 [SMILES: Cc1cccc(C)c1-c1cc2nc(n1)NS(=O)(=O)c1cccc(c1)C(=O)N(Cc1cccc(N3CCC[C@H]3C3CC3)n1)[C@H](CC1CC1)CO2] | | | +++ | | | |
| 1502 | 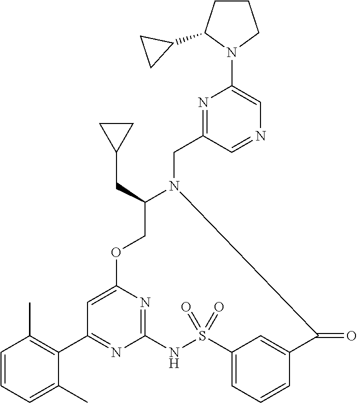 [SMILES: Cc1cccc(C)c1-c1cc2nc(n1)NS(=O)(=O)c1cccc(c1)C(=O)N(Cc1cncc(N3CCC[C@H]3C3CC3)n1)[C@H](CC1CC1)CO2] | +++ | +++ | +++ | | | |
| 1503 | 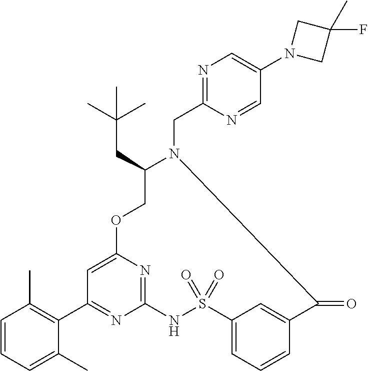 [SMILES: Cc1cccc(C)c1-c1cc2nc(n1)NS(=O)(=O)c1cccc(c1)C(=O)N(Cc1ncc(N3CC(C)(F)C3)cn1)[C@H](CC(C)(C)C)CO2] | +++ | +++ | +++ | | | |
| 1504 | 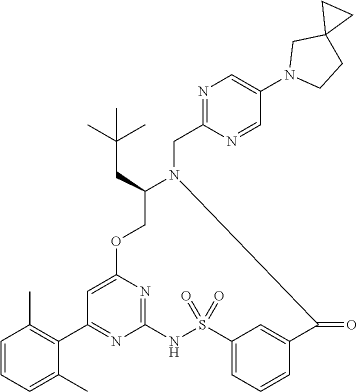 [SMILES: Cc1cccc(C)c1-c1cc2nc(n1)NS(=O)(=O)c1cccc(c1)C(=O)N(Cc1ncc(N3CCC4(CC4)C3)cn1)[C@H](CC(C)(C)C)CO2] | | | +++ | | | |
| 1505 | 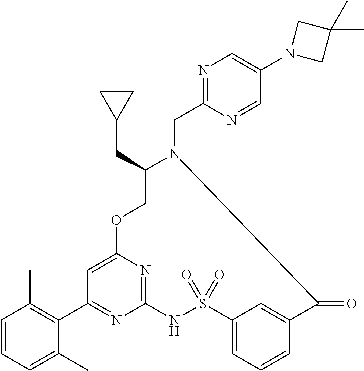 [SMILES: Cc1cccc(C)c1-c1cc2nc(n1)NS(=O)(=O)c1cccc(c1)C(=O)N(Cc1ncc(N3CC(C)(C)C3)cn1)[C@H](CC1CC1)CO2] | +++ | +++ | +++ | | | |
| 1506 | 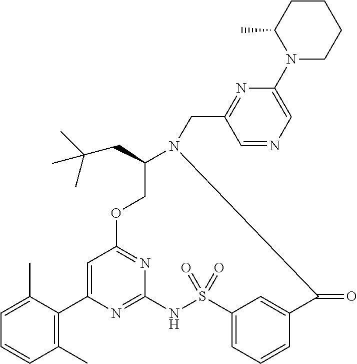 [SMILES: Cc1cccc(C)c1-c1cc2nc(n1)NS(=O)(=O)c1cccc(c1)C(=O)N(Cc1cncc(N3CCCC[C@H]3C)n1)[C@H](CC(C)(C)C)CO2] | | | +++ | +++ | +++ | |
| 1507 | 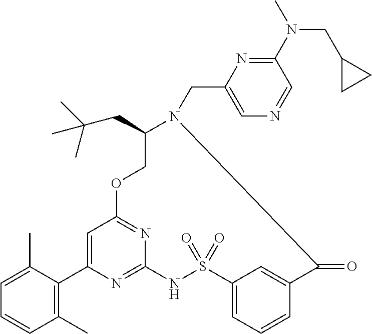 [SMILES: Cc1cccc(C)c1-c1cc2nc(n1)NS(=O)(=O)c1cccc(c1)C(=O)N(Cc1cncc(N(C)CC3CC3)n1)[C@H](CC(C)(C)C)CO2] | | | +++ | | | +++ |
| 1508 | 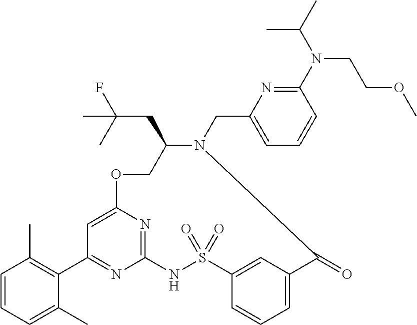 [SMILES: COCCN(c1cccc(CN2C(=O)c3cccc(c3)S(=O)(=O)Nc3nc(cc(-c4c(C)cccc4C)n3)OC[C@H]2CC(C)(C)F)n1)C(C)C] | | | +++ | | | |
| 1509 | 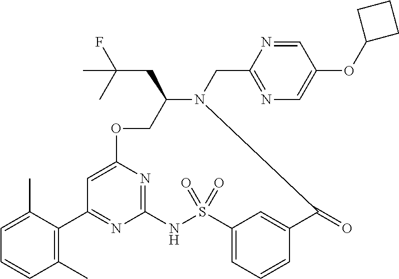 [SMILES: Cc1cccc(C)c1-c1cc2nc(n1)NS(=O)(=O)c1cccc(c1)C(=O)N(Cc1ncc(OC3CCC3)cn1)[C@H](CC(C)(C)F)CO2] | | | +++ | | | |
| 1510 | 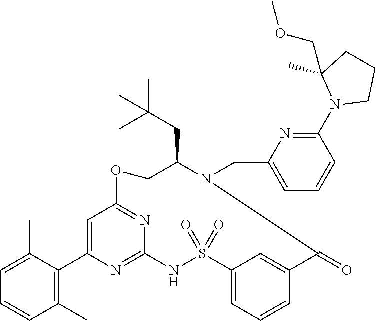 [SMILES: COC[C@@]1(C)CCCN1c1cccc(CN2C(=O)c3cccc(c3)S(=O)(=O)Nc3nc(cc(-c4c(C)cccc4C)n3)OC[C@H]2CC(C)(C)C)n1] | | | +++ | | | |
| 1511 | 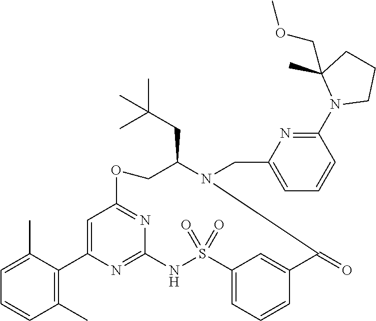 [SMILES: COC[C@]1(C)CCCN1c1cccc(CN2C(=O)c3cccc(c3)S(=O)(=O)Nc3nc(cc(-c4c(C)cccc4C)n3)OC[C@H]2CC(C)(C)C)n1] | | | +++ | | | |
| 1512 | 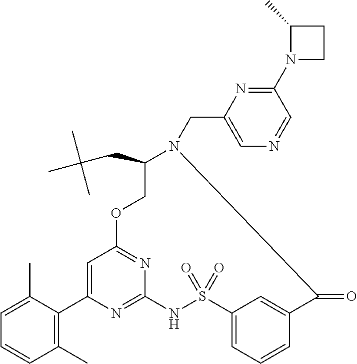 [SMILES: Cc1cccc(C)c1-c1cc2nc(n1)NS(=O)(=O)c1cccc(c1)C(=O)N(Cc1cncc(N3CC[C@H]3C)n1)[C@H](CC(C)(C)C)CO2] | | | +++ | +++ | +++ | |
| 1513 | 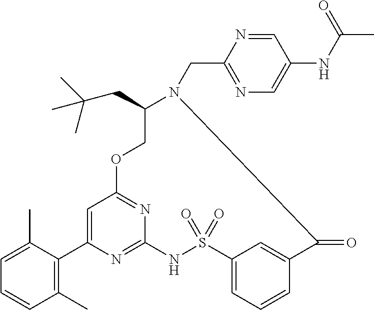 [SMILES: CC(=O)Nc1cnc(CN2C(=O)c3cccc(c3)S(=O)(=O)Nc3nc(cc(-c4c(C)cccc4C)n3)OC[C@H]2CC(C)(C)C)nc1] | | | +++ | | | |
| 1514 | 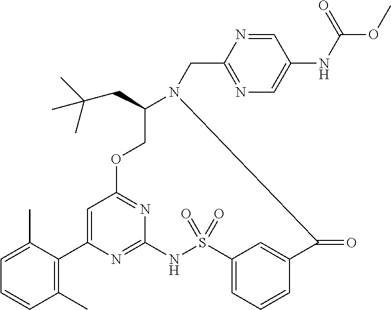 [SMILES: COC(=O)Nc1cnc(CN2C(=O)c3cccc(c3)S(=O)(=O)Nc3nc(cc(-c4c(C)cccc4C)n3)OC[C@H]2CC(C)(C)C)nc1] | | | +++ | | | |
| 1515 | 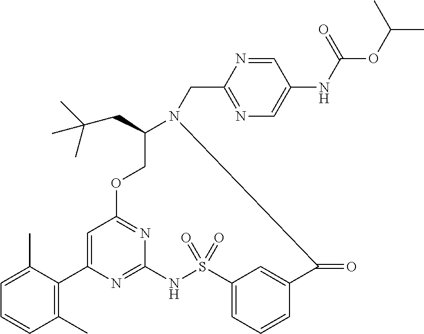 [SMILES: Cc1cccc(C)c1-c1cc2nc(n1)NS(=O)(=O)c1cccc(c1)C(=O)N(Cc1ncc(NC(=O)OC(C)C)cn1)[C@H](CC(C)(C)C)CO2] | | | +++ | | | |
| 1516 | 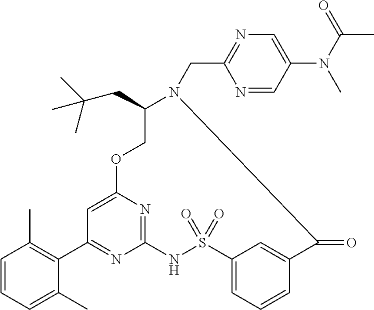 [SMILES: CC(=O)N(C)c1cnc(CN2C(=O)c3cccc(c3)S(=O)(=O)Nc3nc(cc(-c4c(C)cccc4C)n3)OC[C@H]2CC(C)(C)C)nc1] | | | +++ | | | |
| 1517 | 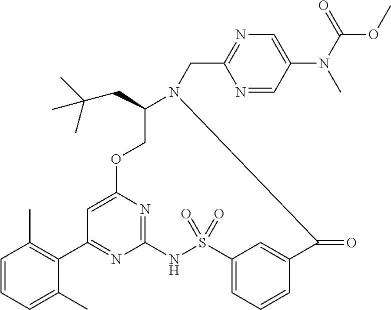 [SMILES: COC(=O)N(C)c1cnc(CN2C(=O)c3cccc(c3)S(=O)(=O)Nc3nc(cc(-c4c(C)cccc4C)n3)OC[C@H]2CC(C)(C)C)nc1] | | | +++ | | | +++ |
| 1518 | 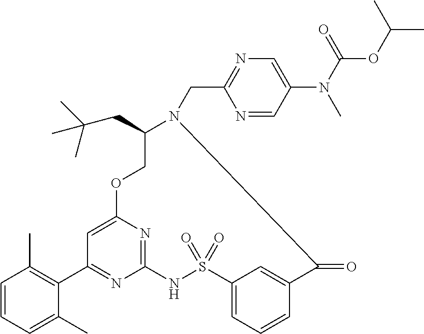 [SMILES: Cc1cccc(C)c1-c1cc2nc(n1)NS(=O)(=O)c1cccc(c1)C(=O)N(Cc1ncc(N(C)C(=O)OC(C)C)cn1)[C@H](CC(C)(C)C)CO2] | | | +++ | | +++ | |
| 1519 | 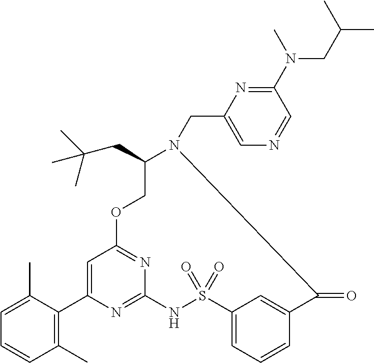 [SMILES: Cc1cccc(C)c1-c1cc2nc(n1)NS(=O)(=O)c1cccc(c1)C(=O)N(Cc1cncc(N(C)CC(C)C)n1)[C@H](CC(C)(C)C)CO2] | | | +++ | +++ | +++ | |
| 1520 | 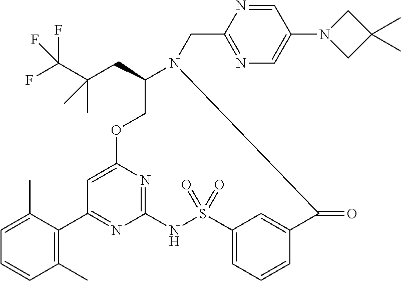 [SMILES: Cc1cccc(C)c1-c1cc2nc(n1)NS(=O)(=O)c1cccc(c1)C(=O)N(Cc1ncc(N3CC(C)(C)C3)cn1)[C@H](CC(C)(C)C(F)(F)F)CO2] | | | +++ | | | +++ |
| 1521 | 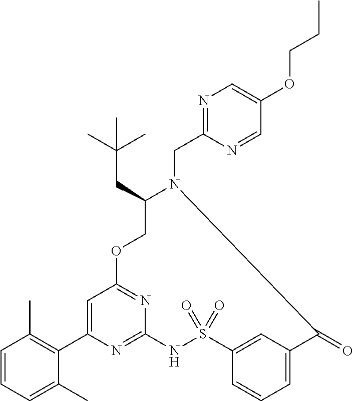 [SMILES: CCCOc1cnc(CN2C(=O)c3cccc(c3)S(=O)(=O)Nc3nc(cc(-c4c(C)cccc4C)n3)OC[C@H]2CC(C)(C)C)nc1] | | | +++ | | | |
| 1522 | 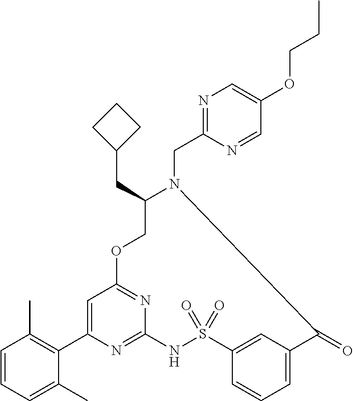 [SMILES: CCCOc1cnc(CN2C(=O)c3cccc(c3)S(=O)(=O)Nc3nc(cc(-c4c(C)cccc4C)n3)OC[C@H]2CC2CCC2)nc1] | | | +++ | +++ | +++ | +++ |
| 1523 | 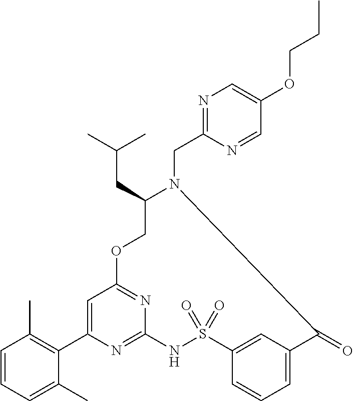 [SMILES: CCCOc1cnc(CN2C(=O)c3cccc(c3)S(=O)(=O)Nc3nc(cc(-c4c(C)cccc4C)n3)OC[C@H]2CC(C)C)nc1] | | | +++ | | | +++ |
| 1524 | 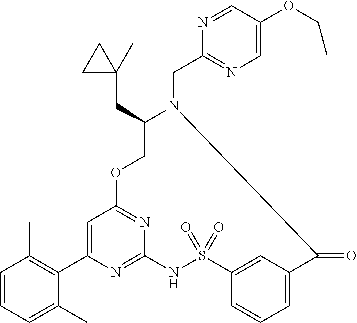 [SMILES: CCOc1cnc(CN2C(=O)c3cccc(c3)S(=O)(=O)Nc3nc(cc(-c4c(C)cccc4C)n3)OC[C@H]2CC2(C)CC2)nc1] | | | +++ | | | |
| 1306 | 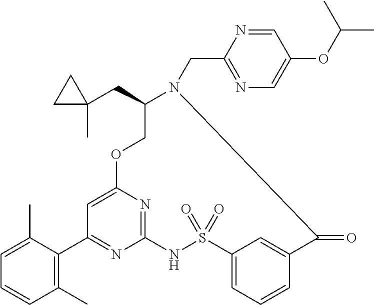 [SMILES: Cc1cccc(C)c1-c1cc2nc(n1)NS(=O)(=O)c1cccc(c1)C(=O)N(Cc1ncc(OC(C)C)cn1)[C@H](CC1(C)CC1)CO2] | | | +++ | +++ | +++ | +++ |
| 1307 | 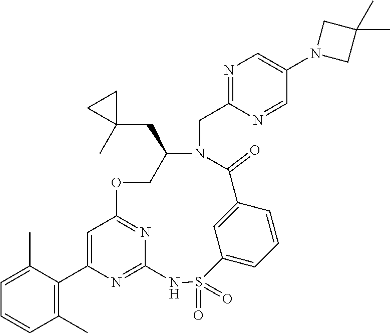 [SMILES: Cc1cccc(C)c1-c1cc2nc(n1)NS(=O)(=O)c1cccc(c1)C(=O)N(Cc1ncc(N3CC(C)(C)C3)cn1)[C@H](CC1(C)CC1)CO2] | | | +++ | | | +++ |
| 1308 | 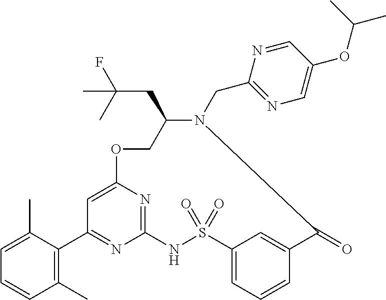 [SMILES: Cc1cccc(C)c1-c1cc2nc(n1)NS(=O)(=O)c1cccc(c1)C(=O)N(Cc1ncc(OC(C)C)cn1)[C@H](CC(C)(C)F)CO2] | | | +++ | | | |
| 1525 | 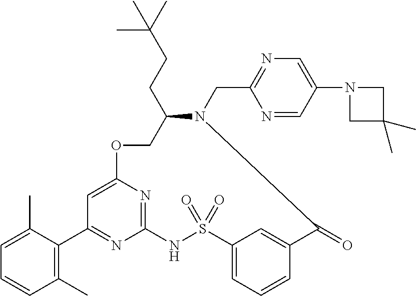 [SMILES: Cc1cccc(C)c1-c1cc2nc(n1)NS(=O)(=O)c1cccc(c1)C(=O)N(Cc1ncc(N3CC(C)(C)C3)cn1)[C@H](CCC(C)(C)C)CO2] | | | +++ | | | |
| 1309 | 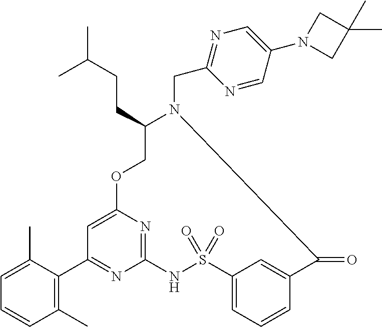 [SMILES: Cc1cccc(C)c1-c1cc2nc(n1)NS(=O)(=O)c1cccc(c1)C(=O)N(Cc1ncc(N3CC(C)(C)C3)cn1)[C@H](CCC(C)C)CO2] | | | +++ | | | |
| 1526 | 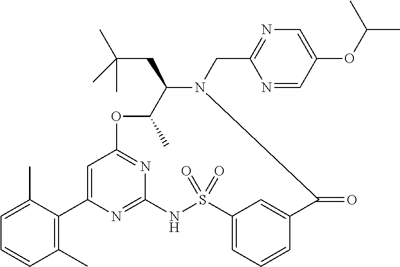 [SMILES: Cc1cccc(C)c1-c1cc2nc(n1)NS(=O)(=O)c1cccc(c1)C(=O)N(Cc1ncc(OC(C)C)cn1)[C@H](CC(C)(C)C)[C@H](C)O2] | | | +++ | | | |
| 1527 | 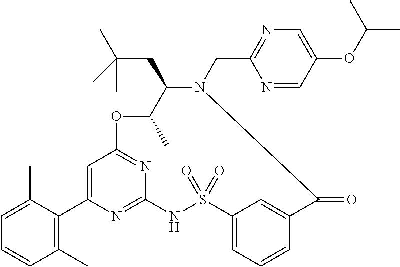 [SMILES: Cc1cccc(C)c1-c1cc2nc(n1)NS(=O)(=O)c1cccc(c1)C(=O)N(Cc1ncc(OC(C)C)cn1)[C@H](CC(C)(C)C)[C@H](C)O2] | | | +++ | | | |
| 1528 | 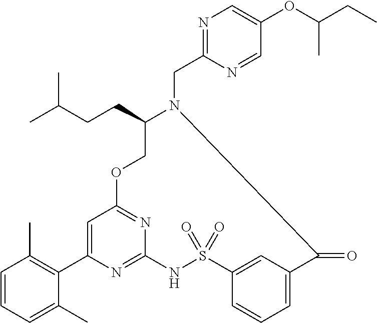 [SMILES: CCC(C)Oc1cnc(CN2C(=O)c3cccc(c3)S(=O)(=O)Nc3nc(cc(-c4c(C)cccc4C)n3)OC[C@H]2CCC(C)C)nc1] | | | +++ | | | +++ |
| 1529 | 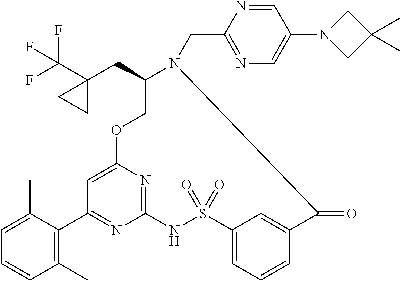 [SMILES: Cc1cccc(C)c1-c1cc2nc(n1)NS(=O)(=O)c1cccc(c1)C(=O)N(Cc1ncc(N3CC(C)(C)C3)cn1)[C@H](CC1(C(F)(F)F)CC1)CO2] | | | +++ | | | +++ |
| 1530 | 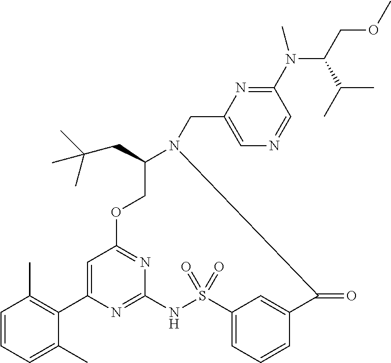 [SMILES: COC[C@H](C(C)C)N(C)c1cncc(CN2C(=O)c3cccc(c3)S(=O)(=O)Nc3nc(cc(-c4c(C)cccc4C)n3)OC[C@H]2CC(C)(C)C)n1] | | | +++ | | | |
| 1531 | 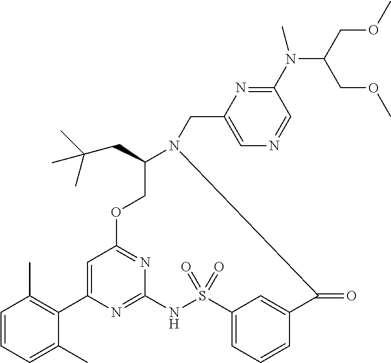 [SMILES: COCC(COC)N(C)c1cncc(CN2C(=O)c3cccc(c3)S(=O)(=O)Nc3nc(cc(-c4c(C)cccc4C)n3)OC[C@H]2CC(C)(C)C)n1] | | | +++ | | | |
| 1532 | 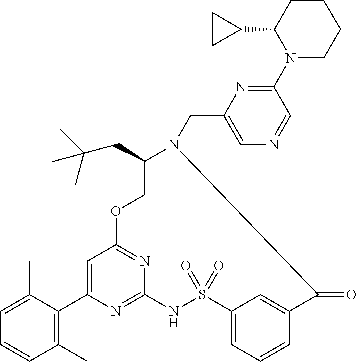 [SMILES: Cc1cccc(C)c1-c1cc2nc(n1)NS(=O)(=O)c1cccc(c1)C(=O)N(Cc1cncc(N3CCCC[C@H]3C3CC3)n1)[C@H](CC(C)(C)C)CO2] | | | +++ | | | |
| 1533 | 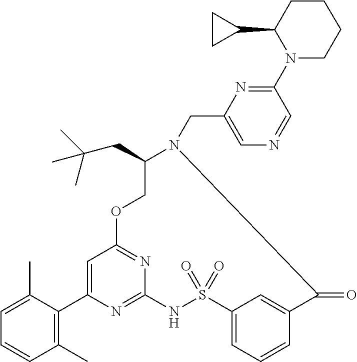 [SMILES: Cc1cccc(C)c1-c1cc2nc(n1)NS(=O)(=O)c1cccc(c1)C(=O)N(Cc1cncc(N3CCCC[C@@H]3C3CC3)n1)[C@H](CC(C)(C)C)CO2] | | | +++ | | | |
| 1534 | 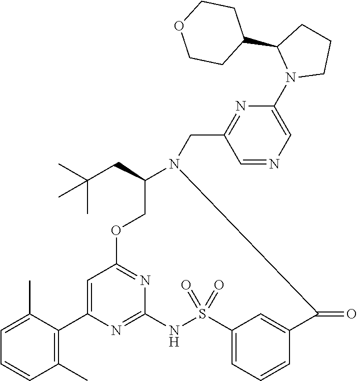 [SMILES: Cc1cccc(C)c1-c1cc2nc(n1)NS(=O)(=O)c1cccc(c1)C(=O)N(Cc1cncc(N3CCC[C@@H]3C3CCOCC3)n1)[C@H](CC(C)(C)C)CO2] | | | +++ | | | |
| 1535 | 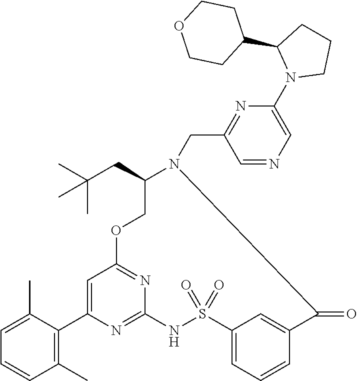 [SMILES: Cc1cccc(C)c1-c1cc2nc(n1)NS(=O)(=O)c1cccc(c1)C(=O)N(Cc1cncc(N3CCC[C@@H]3C3CCOCC3)n1)[C@H](CC(C)(C)C)CO2] | | | +++ | | | |
| 1536 | 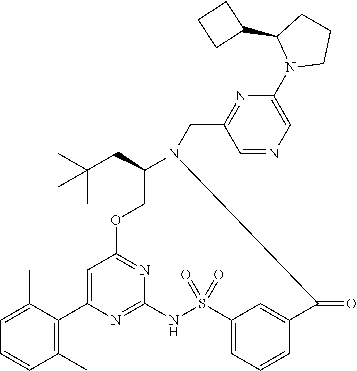 [SMILES: Cc1cccc(C)c1-c1cc2nc(n1)NS(=O)(=O)c1cccc(c1)C(=O)N(Cc1cncc(N3CCC[C@@H]3C3CCC3)n1)[C@H](CC(C)(C)C)CO2] | | | +++ | | | |
| 1537 | 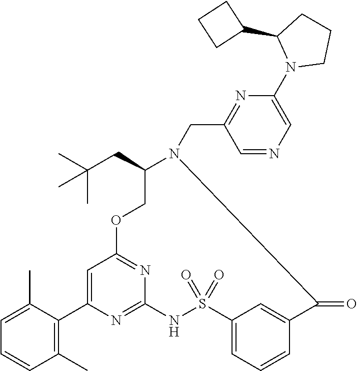 [SMILES: Cc1cccc(C)c1-c1cc2nc(n1)NS(=O)(=O)c1cccc(c1)C(=O)N(Cc1cncc(N3CCC[C@@H]3C3CCC3)n1)[C@H](CC(C)(C)C)CO2] | | | +++ | | | |
| 1538 | 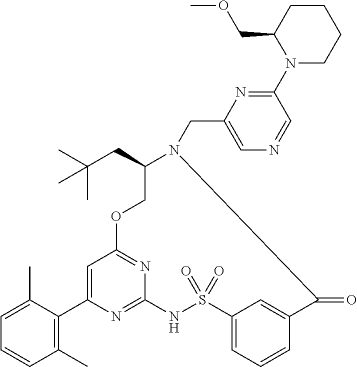 [SMILES: COC[C@H]1CCCCN1c1cncc(CN2C(=O)c3cccc(c3)S(=O)(=O)Nc3nc(cc(-c4c(C)cccc4C)n3)OC[C@H]2CC(C)(C)C)n1] | | | +++ | | | |
| 1539 | 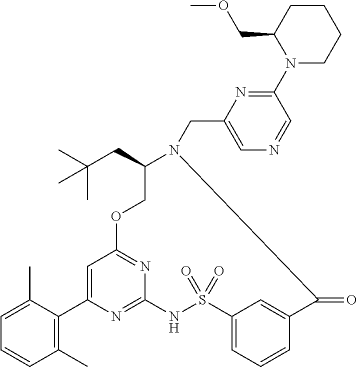 [SMILES: COC[C@H]1CCCCN1c1cncc(CN2C(=O)c3cccc(c3)S(=O)(=O)Nc3nc(cc(-c4c(C)cccc4C)n3)OC[C@H]2CC(C)(C)C)n1] | | | +++ | +++ | +++ | |
| 1540 | 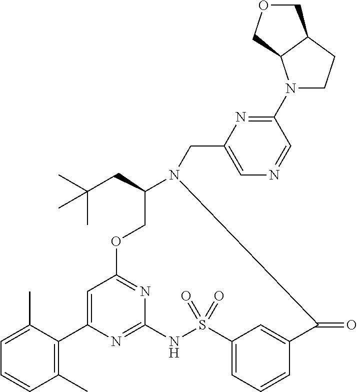 [SMILES: Cc1cccc(C)c1-c1cc2nc(n1)NS(=O)(=O)c1cccc(c1)C(=O)N(Cc1cncc(N3CC[C@@H]4COC[C@@H]43)n1)[C@H](CC(C)(C)C)CO2] | | | +++ | | | |
| 1541 | 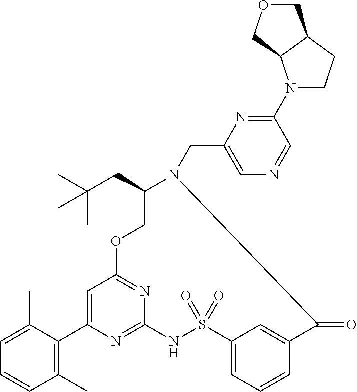 [SMILES: Cc1cccc(C)c1-c1cc2nc(n1)NS(=O)(=O)c1cccc(c1)C(=O)N(Cc1cncc(N3CC[C@@H]4COC[C@@H]43)n1)[C@H](CC(C)(C)C)CO2] | | | +++ | +++ | +++ | |
| 1542 | 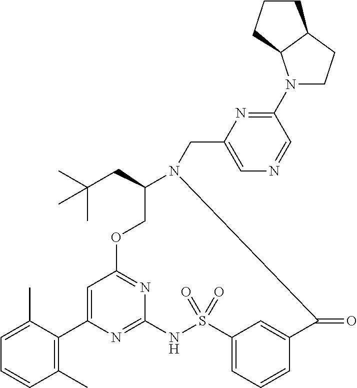 [SMILES: Cc1cccc(C)c1-c1cc2nc(n1)NS(=O)(=O)c1cccc(c1)C(=O)N(Cc1cncc(N3CC[C@@H]4CCC[C@@H]43)n1)[C@H](CC(C)(C)C)CO2] | | | +++ | | | |
| 1543 | 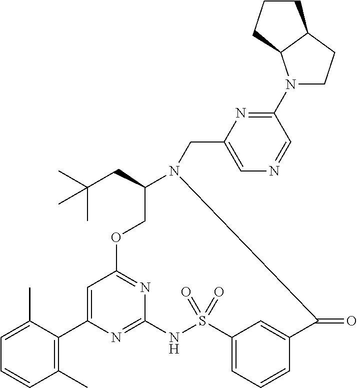 [SMILES: Cc1cccc(C)c1-c1cc2nc(n1)NS(=O)(=O)c1cccc(c1)C(=O)N(Cc1cncc(N3CC[C@@H]4CCC[C@@H]43)n1)[C@H](CC(C)(C)C)CO2] | | | +++ | | | |
| 1544 | 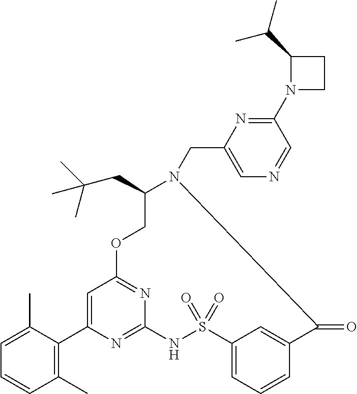 [SMILES: Cc1cccc(C)c1-c1cc2nc(n1)NS(=O)(=O)c1cccc(c1)C(=O)N(Cc1cncc(N3CC[C@@H]3C(C)C)n1)[C@H](CC(C)(C)C)CO2] | | | +++ | | | |
| 1545 | 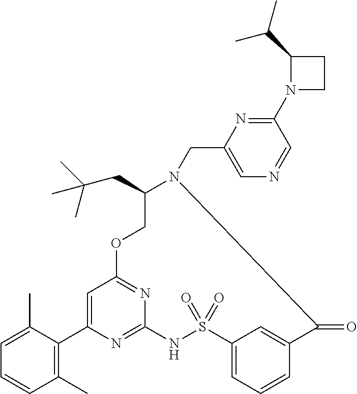 [SMILES: Cc1cccc(C)c1-c1cc2nc(n1)NS(=O)(=O)c1cccc(c1)C(=O)N(Cc1cncc(N3CC[C@@H]3C(C)C)n1)[C@H](CC(C)(C)C)CO2] | | | +++ | +++ | +++ | |
| 1546 | 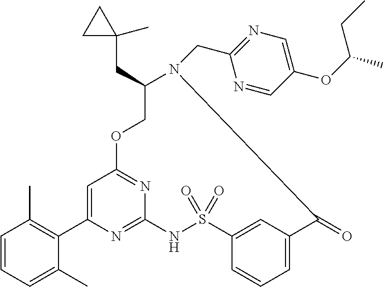 [SMILES: CC[C@H](C)Oc1cnc(CN2C(=O)c3cccc(c3)S(=O)(=O)Nc3nc(cc(-c4c(C)cccc4C)n3)OC[C@H]2CC2(C)CC2)nc1] | | | +++ | | | +++ |
| 1547 | 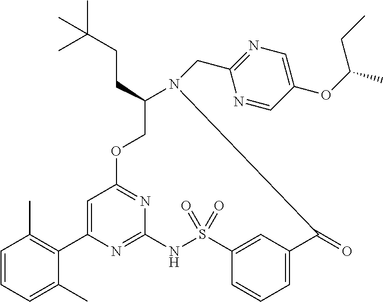 [SMILES: CC[C@H](C)Oc1cnc(CN2C(=O)c3cccc(c3)S(=O)(=O)Nc3nc(cc(-c4c(C)cccc4C)n3)OC[C@H]2CCC(C)(C)C)nc1] | | | +++ | | | +++ |
| 1548 | 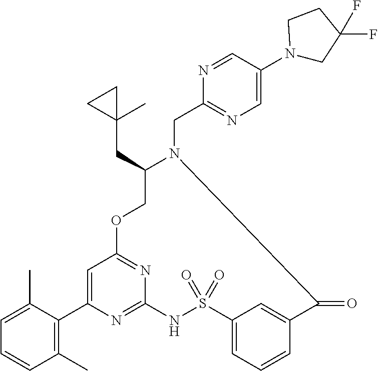 [SMILES: Cc1cccc(C)c1-c1cc2nc(n1)NS(=O)(=O)c1cccc(c1)C(=O)N(Cc1ncc(N3CCC(F)(F)C3)cn1)[C@H](CC1(C)CC1)CO2] | | | | | | +++ |
| 1549 | 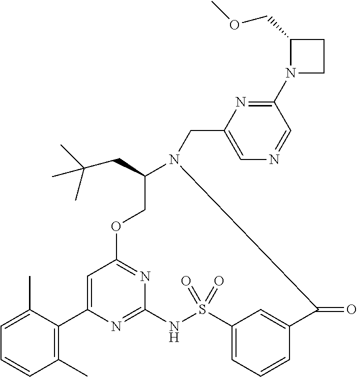 [SMILES: COC[C@@H]1CCN1c1cncc(CN2C(=O)c3cccc(c3)S(=O)(=O)Nc3nc(cc(-c4c(C)cccc4C)n3)OC[C@H]2CC(C)(C)C)n1] | | | | +++ | +++ | +++ |
| 1550 | 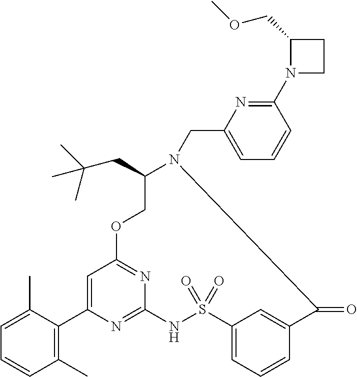 [SMILES: COC[C@@H]1CCN1c1cccc(CN2C(=O)c3cccc(c3)S(=O)(=O)Nc3nc(cc(-c4c(C)cccc4C)n3)OC[C@H]2CC(C)(C)C)n1] | | | | | | +++ |
| 1551 | 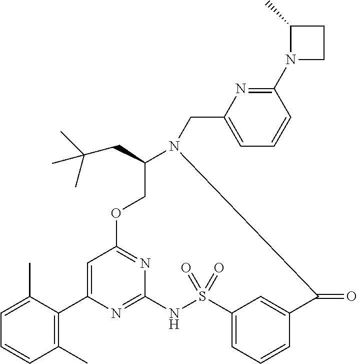 [SMILES: Cc1cccc(C)c1-c1cc2nc(n1)NS(=O)(=O)c1cccc(c1)C(=O)N(Cc1cccc(N3CC[C@H]3C)n1)[C@H](CC(C)(C)C)CO2] | | | | | | +++ |
| 1552 | 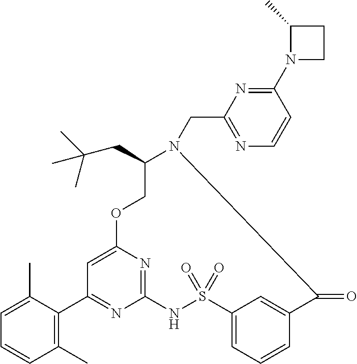 [SMILES: Cc1cccc(C)c1-c1cc2nc(n1)NS(=O)(=O)c1cccc(c1)C(=O)N(Cc1nccc(N3CC[C@H]3C)n1)[C@H](CC(C)(C)C)CO2] | | | | | | +++ |
| 1553 | 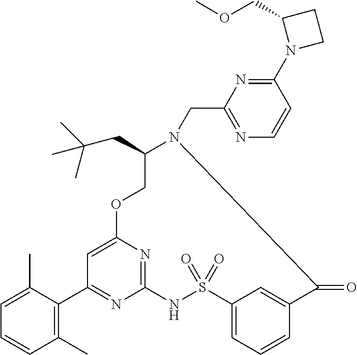 [SMILES: COC[C@@H]1CCN1c1ccnc(CN2C(=O)c3cccc(c3)S(=O)(=O)Nc3nc(cc(-c4c(C)cccc4C)n3)OC[C@H]2CC(C)(C)C)n1] | | | | | | +++ |
| 1554 | 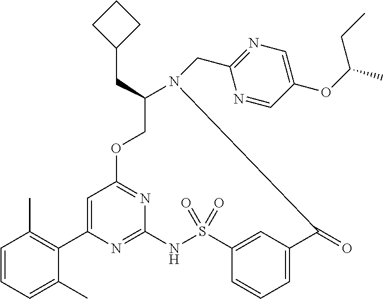 [SMILES: CC[C@H](C)Oc1cnc(CN2C(=O)c3cccc(c3)S(=O)(=O)Nc3nc(cc(-c4c(C)cccc4C)n3)OC[C@H]2CC2CCC2)nc1] | | | | | | +++ |
| 1555 | 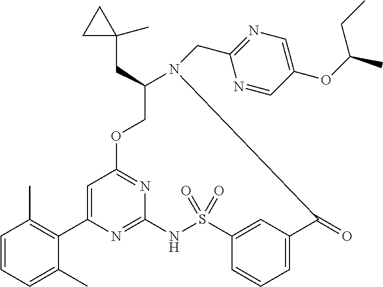 [SMILES: CC[C@@H](C)Oc1cnc(CN2C(=O)c3cccc(c3)S(=O)(=O)Nc3nc(cc(-c4c(C)cccc4C)n3)OC[C@H]2CC2(C)CC2)nc1] | | | | | | +++ |
| 1556 | 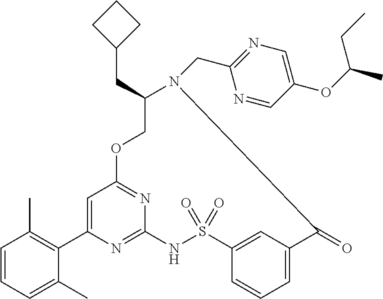 [SMILES: CC[C@@H](C)Oc1cnc(CN2C(=O)c3cccc(c3)S(=O)(=O)Nc3nc(cc(-c4c(C)cccc4C)n3)OC[C@H]2CC2CCC2)nc1] | | | | | | +++ |
| 1557 | 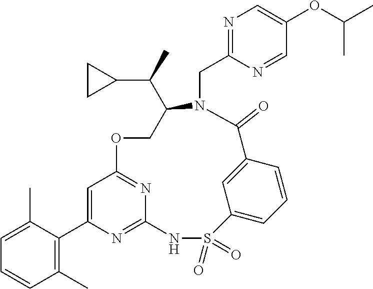 [SMILES: Cc1cccc(C)c1-c1cc2nc(n1)NS(=O)(=O)c1cccc(c1)C(=O)N(Cc1ncc(OC(C)C)cn1)[C@H]([C@H](C)C1CC1)CO2] | | | | | | + |
| 1558 | 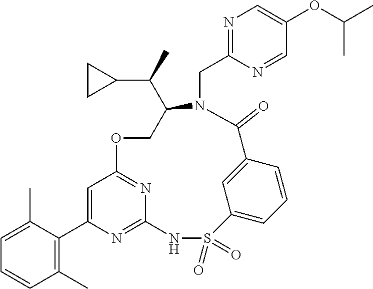 [SMILES: Cc1cccc(C)c1-c1cc2nc(n1)NS(=O)(=O)c1cccc(c1)C(=O)N(Cc1ncc(OC(C)C)cn1)[C@H]([C@H](C)C1CC1)CO2] | | | | | | +++ |
| 1559 | 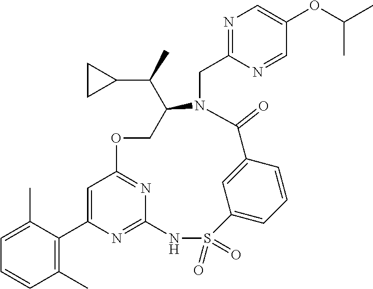 [SMILES: Cc1cccc(C)c1-c1cc2nc(n1)NS(=O)(=O)c1cccc(c1)C(=O)N(Cc1ncc(OC(C)C)cn1)[C@H]([C@H](C)C1CC1)CO2] | | | | | | +++ |
| 1560 | 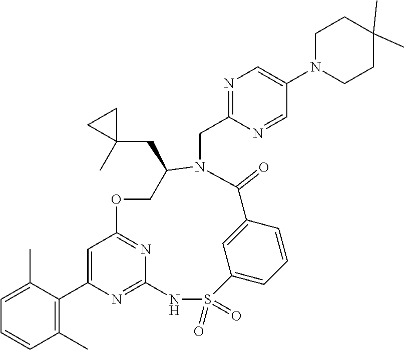 [SMILES: Cc1cccc(C)c1-c1cc2nc(n1)NS(=O)(=O)c1cccc(c1)C(=O)N(Cc1ncc(N3CCC(C)(C)CC3)cn1)[C@H](CC1(C)CC1)CO2] | | | | | | +++ |
| 1561 | 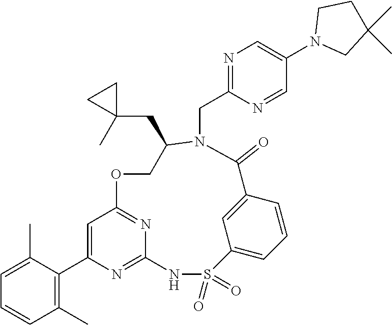 [SMILES: Cc1cccc(C)c1-c1cc2nc(n1)NS(=O)(=O)c1cccc(c1)C(=O)N(Cc1ncc(N3CCC(C)(C)C3)cn1)[C@H](CC1(C)CC1)CO2] | | | | | | +++ |
| 1562 | 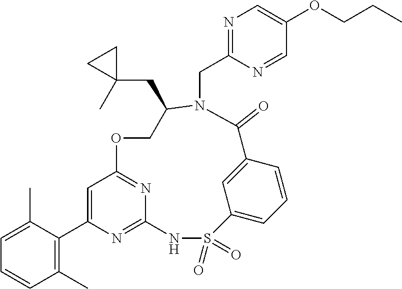 [SMILES: CCCOc1cnc(CN2C(=O)c3cccc(c3)S(=O)(=O)Nc3nc(cc(-c4c(C)cccc4C)n3)OC[C@H]2CC2(C)CC2)nc1] | | | | | | +++ |
| 1563 | 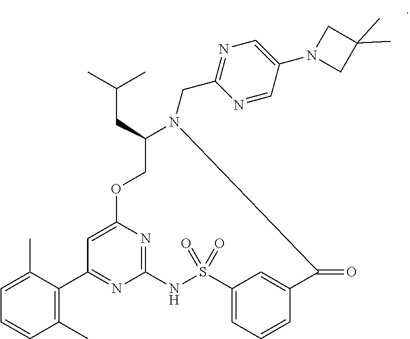 [SMILES: Cc1cccc(C)c1-c1cc2nc(n1)NS(=O)(=O)c1cccc(c1)C(=O)N(Cc1ncc(N3CC(C)(C)C3)cn1)[C@H](CC(C)C)CO2] | | | | | | +++ |
| 1564 | 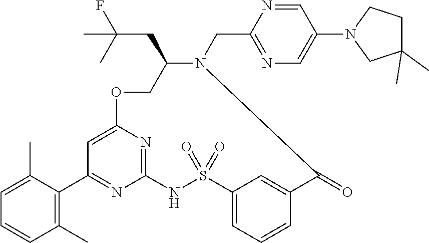 [SMILES: Cc1cccc(C)c1-c1cc2nc(n1)NS(=O)(=O)c1cccc(c1)C(=O)N(Cc1ncc(N3CCC(C)(C)C3)cn1)[C@H](CC(C)(C)F)CO2] | | | | | | +++ |
| 1565 | 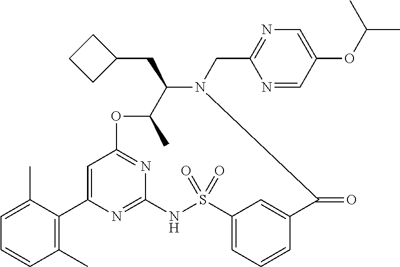 [SMILES: Cc1cccc(C)c1-c1cc2nc(n1)NS(=O)(=O)c1cccc(c1)C(=O)N(Cc1ncc(OC(C)C)cn1)[C@H](CC1CCC1)[C@@H](C)O2] | | | | | | +++ |
| 1566 | 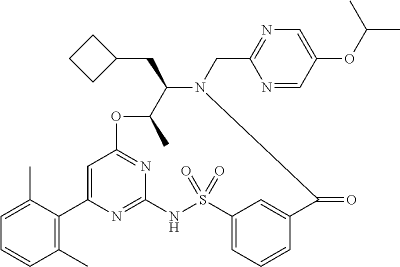 [SMILES: Cc1cccc(C)c1-c1cc2nc(n1)NS(=O)(=O)c1cccc(c1)C(=O)N(Cc1ncc(OC(C)C)cn1)[C@H](CC1CCC1)[C@@H](C)O2] | | | | | | + |
| 1567 | 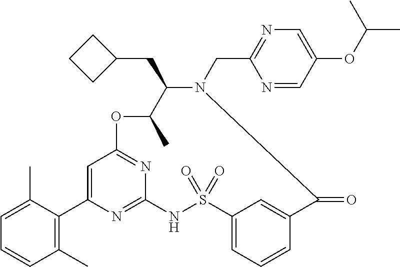 [SMILES: Cc1cccc(C)c1-c1cc2nc(n1)NS(=O)(=O)c1cccc(c1)C(=O)N(Cc1ncc(OC(C)C)cn1)[C@H](CC1CCC1)[C@@H](C)O2] | | | | | | +++ |
| 1568 | 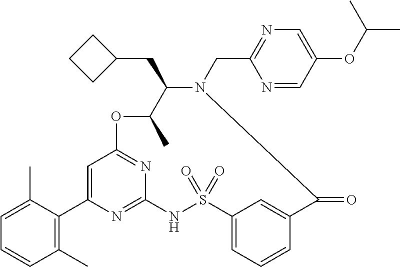 [SMILES: Cc1cccc(C)c1-c1cc2nc(n1)NS(=O)(=O)c1cccc(c1)C(=O)N(Cc1ncc(OC(C)C)cn1)[C@H](CC1CCC1)[C@@H](C)O2] | | | | | | +++ |
| 1569 | 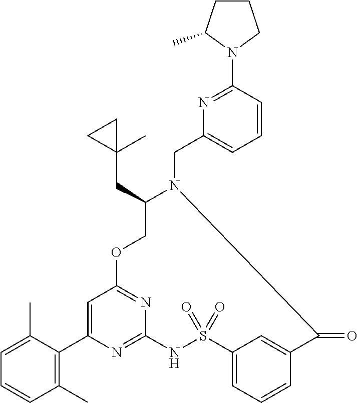 [SMILES: Cc1cccc(C)c1-c1cc2nc(n1)NS(=O)(=O)c1cccc(c1)C(=O)N(Cc1cccc(N3CCC[C@H]3C)n1)[C@H](CC1(C)CC1)CO2] | | | | | | +++ |
| 1570 | 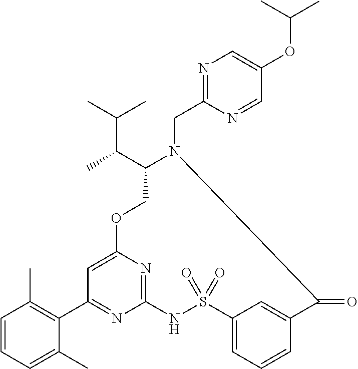 [SMILES: Cc1cccc(C)c1-c1cc2nc(n1)NS(=O)(=O)c1cccc(c1)C(=O)N(Cc1ncc(OC(C)C)cn1)[C@@H]([C@H](C)C(C)C)CO2] | | | | | | + |
| 1571 | 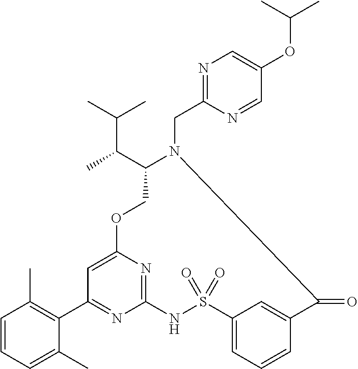 [SMILES: Cc1cccc(C)c1-c1cc2nc(n1)NS(=O)(=O)c1cccc(c1)C(=O)N(Cc1ncc(OC(C)C)cn1)[C@@H]([C@H](C)C(C)C)CO2] | | | | | | + |
| 1572 | 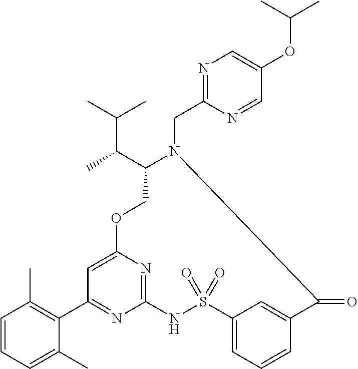 [SMILES: Cc1cccc(C)c1-c1cc2nc(n1)NS(=O)(=O)c1cccc(c1)C(=O)N(Cc1ncc(OC(C)C)cn1)[C@@H]([C@H](C)C(C)C)CO2] | | | | | | +++ |
| 1573 | 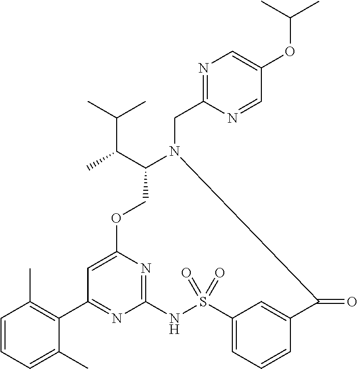 [SMILES: Cc1cccc(C)c1-c1cc2nc(n1)NS(=O)(=O)c1cccc(c1)C(=O)N(Cc1ncc(OC(C)C)cn1)[C@@H]([C@H](C)C(C)C)CO2] | | | | | | +++ |
| 1574 | 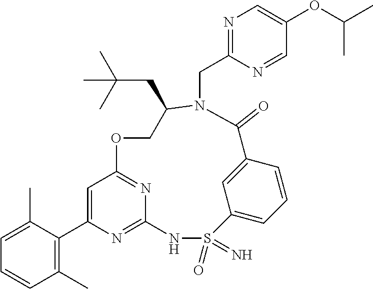 [SMILES: Cc1cccc(C)c1-c1cc2nc(n1)NS(=N)(=O)c1cccc(c1)C(=O)N(Cc1ncc(OC(C)C)cn1)[C@H](CC(C)(C)C)CO2] | | | | | | ++ |
| 1575 | 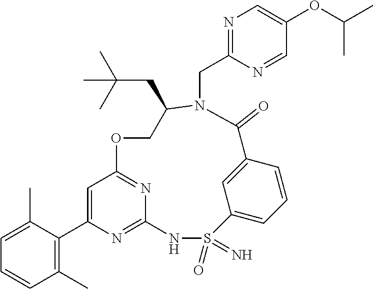 [SMILES: Cc1cccc(C)c1-c1cc2nc(n1)NS(=N)(=O)c1cccc(c1)C(=O)N(Cc1ncc(OC(C)C)cn1)[C@H](CC(C)(C)C)CO2] | | | | | | +++ |
| 1576 | 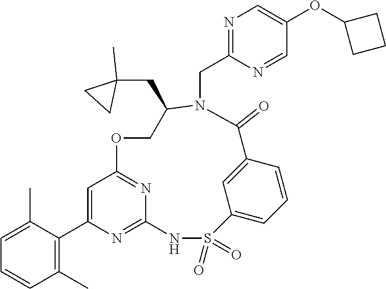 [SMILES: Cc1cccc(C)c1-c1cc2nc(n1)NS(=O)(=O)c1cccc(c1)C(=O)N(Cc1ncc(OC3CCC3)cn1)[C@H](CC1(C)CC1)CO2] | | | | | | +++ |
| 1577 | 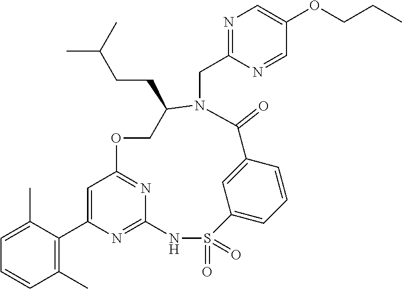 [SMILES: CCCOc1cnc(CN2C(=O)c3cccc(c3)S(=O)(=O)Nc3nc(cc(-c4c(C)cccc4C)n3)OC[C@H]2CCC(C)C)nc1] | | | | | | +++ |
| 1310 | 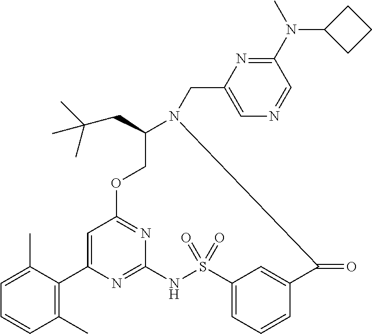 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)C)CO2] | | | | +++ | +++ | +++ |
| 1578 | 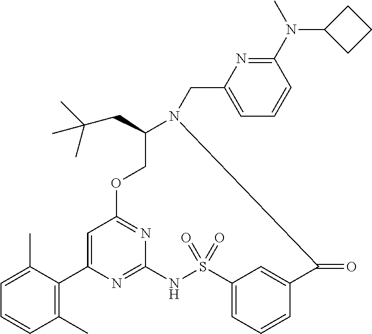 [SMILES: Cc1cccc(C)c1-c1cc2nc(n1)NS(=O)(=O)c1cccc(c1)C(=O)N(Cc1cccc(N(C)C3CCC3)n1)[C@H](CC(C)(C)C)CO2] | | | | +++ | +++ | +++ |
| 1579 | 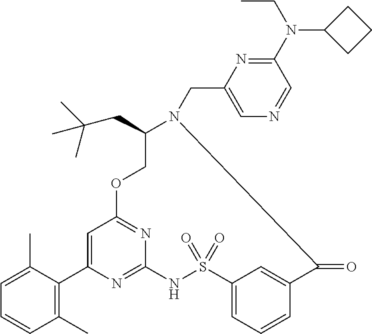 [SMILES: CCN(c1cncc(CN2C(=O)c3cccc(c3)S(=O)(=O)Nc3nc(cc(-c4c(C)cccc4C)n3)OC[C@H]2CC(C)(C)C)n1)C1CCC1] | | | | +++ | +++ | +++ |
| 1580 | 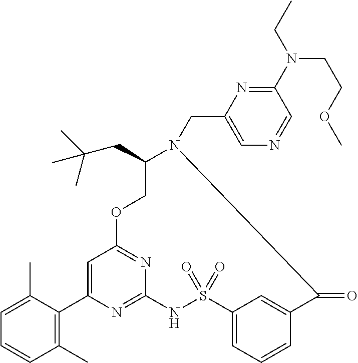 [SMILES: CCN(CCOC)c1cncc(CN2C(=O)c3cccc(c3)S(=O)(=O)Nc3nc(cc(-c4c(C)cccc4C)n3)OC[C@H]2CC(C)(C)C)n1] | | | | +++ | +++ | +++ |
| 1581 | 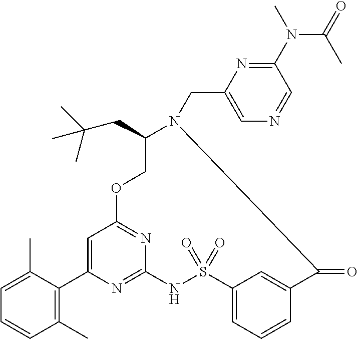 [SMILES: CC(=O)N(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1582 | 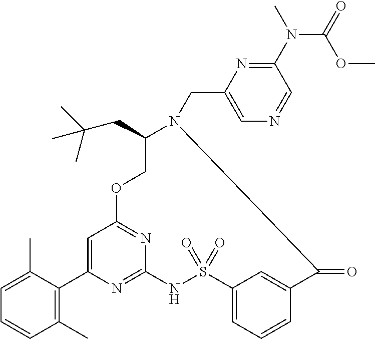 [SMILES: COC(=O)N(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1583 | 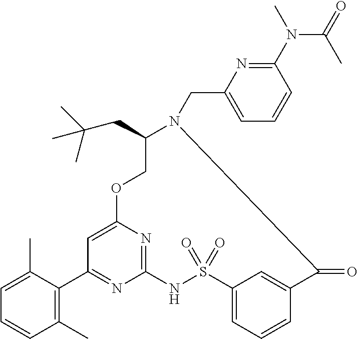 [SMILES: CC(=O)N(C)c1cccc(CN2C(=O)c3cccc(c3)S(=O)(=O)Nc3nc(cc(-c4c(C)cccc4C)n3)OC[C@H]2CC(C)(C)C)n1] | | | | | | +++ |
| 1584 | 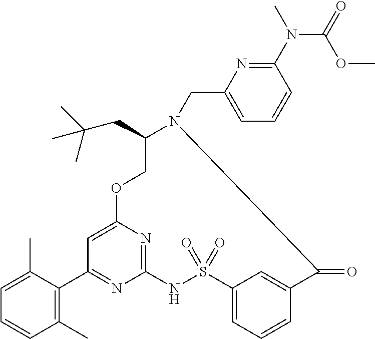 [SMILES: COC(=O)N(C)c1cccc(CN2C(=O)c3cccc(c3)S(=O)(=O)Nc3nc(cc(-c4c(C)cccc4C)n3)OC[C@H]2CC(C)(C)C)n1] | | | | | | +++ |
| 1342 | 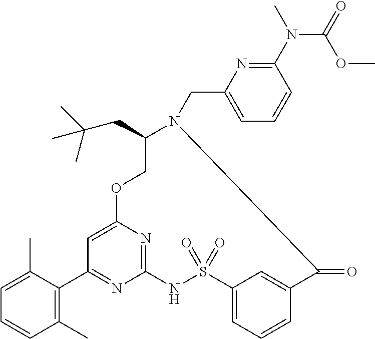 [SMILES: COC(=O)N(C)c1cccc(CN2C(=O)c3cccc(c3)S(=O)(=O)Nc3nc(cc(-c4c(C)cccc4C)n3)OC[C@H]2CC(C)(C)C)n1] | | | | | | +++ |
| 1311 | 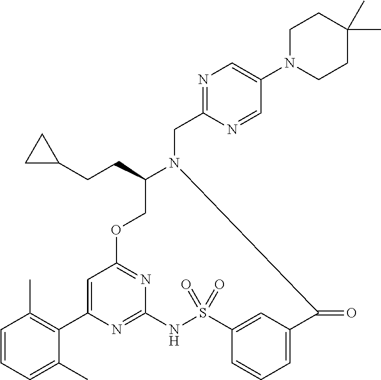 [SMILES: Cc1cccc(C)c1-c1cc2nc(n1)NS(=O)(=O)c1cccc(c1)C(=O)N(Cc1ncc(N3CCC(C)(C)CC3)cn1)[C@H](CCC1CC1)CO2] | | | | | | +++ |
| 1585 | 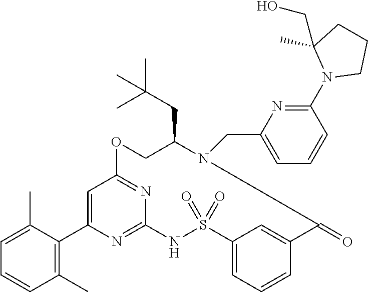 [SMILES: Cc1cccc(C)c1-c1cc2nc(n1)NS(=O)(=O)c1cccc(c1)C(=O)N(Cc1cccc(N3CCC[C@]3(C)CO)n1)[C@H](CC(C)(C)C)CO2] | | | | | | +++ |
| 1586 | 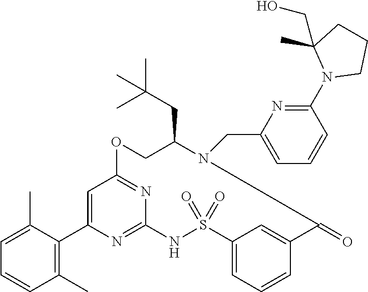 [SMILES: Cc1cccc(C)c1-c1cc2nc(n1)NS(=O)(=O)c1cccc(c1)C(=O)N(Cc1cccc(N3CCC[C@@]3(C)CO)n1)[C@H](CC(C)(C)C)CO2] | | | | | | +++ |
| 1587 | 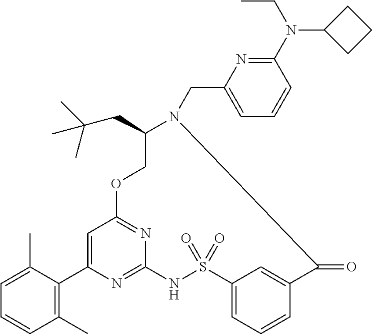 [SMILES: CCN(c1cccc(CN2C(=O)c3cccc(c3)S(=O)(=O)Nc3nc(cc(-c4c(C)cccc4C)n3)OC[C@H]2CC(C)(C)C)n1)C1CCC1] | | | | +++ | +++ | +++ |
| 1588 | 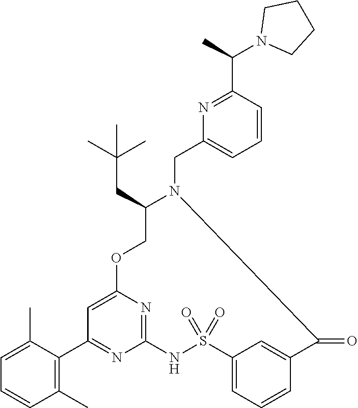 [SMILES: Cc1cccc(C)c1-c1cc2nc(n1)NS(=O)(=O)c1cccc(c1)C(=O)N(Cc1cccc([C@@H](C)N3CCCC3)n1)[C@H](CC(C)(C)C)CO2] | | | | | | +++ |
| 1589 | 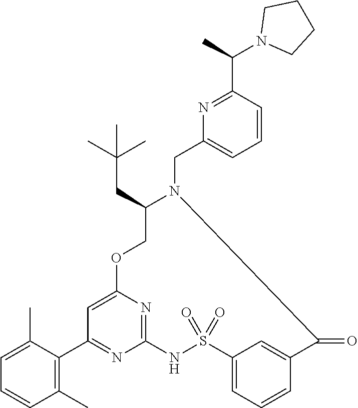 [SMILES: Cc1cccc(C)c1-c1cc2nc(n1)NS(=O)(=O)c1cccc(c1)C(=O)N(Cc1cccc([C@@H](C)N3CCCC3)n1)[C@H](CC(C)(C)C)CO2] | | | | | | ++ |
| 1590 | 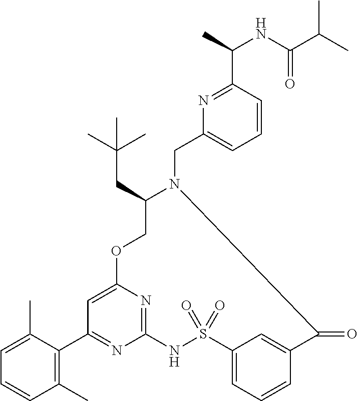 [SMILES: Cc1cccc(C)c1-c1cc2nc(n1)NS(=O)(=O)c1cccc(c1)C(=O)N(Cc1cccc([C@@H](C)NC(=O)C(C)C)n1)[C@H](CC(C)(C)C)CO2] | | | | | | +++ |
| 1591 | 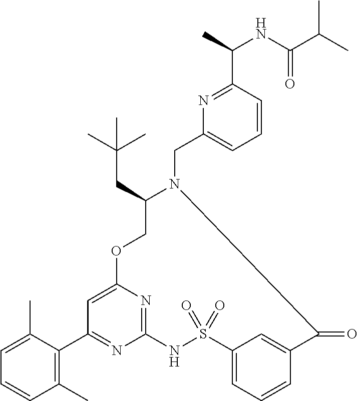 [SMILES: Cc1cccc(C)c1-c1cc2nc(n1)NS(=O)(=O)c1cccc(c1)C(=O)N(Cc1cccc([C@@H](C)NC(=O)C(C)C)n1)[C@H](CC(C)(C)C)CO2] | | | | | | +++ |
| 1592 | 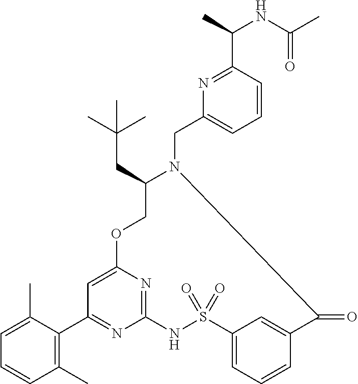 [SMILES: CC(=O)N[C@H](C)c1cccc(CN2C(=O)c3cccc(c3)S(=O)(=O)Nc3nc(cc(-c4c(C)cccc4C)n3)OC[C@H]2CC(C)(C)C)n1] | | | | | | +++ |
| 1593 | 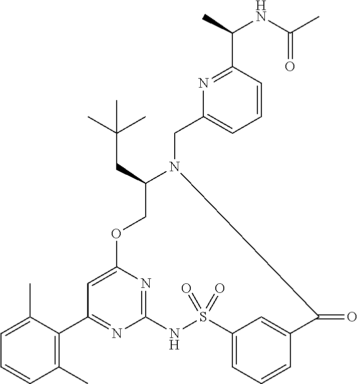 [SMILES: CC(=O)N[C@H](C)c1cccc(CN2C(=O)c3cccc(c3)S(=O)(=O)Nc3nc(cc(-c4c(C)cccc4C)n3)OC[C@H]2CC(C)(C)C)n1] | | | | | | +++ |
| 1298 | 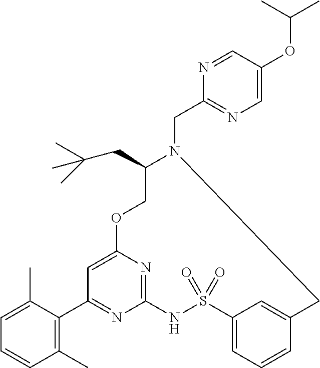 [SMILES: Cc1cccc(C)c1-c1cc2nc(n1)NS(=O)(=O)c1cccc(c1)CN(Cc1ncc(OC(C)C)cn1)[C@H](CC(C)(C)C)CO2] | | | | | | ++ |
| 1594 | 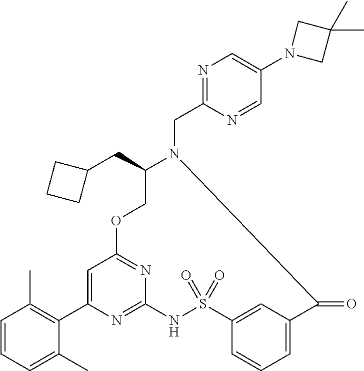 [SMILES: Cc1cccc(C)c1-c1cc2nc(n1)NS(=O)(=O)c1cccc(c1)C(=O)N(Cc1ncc(N3CC(C)(C)C3)cn1)[C@H](CC1CCC1)CO2] | | | | +++ | +++ | |
| 1595 | 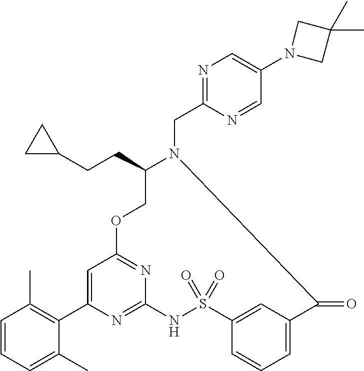 [SMILES: Cc1cccc(C)c1-c1cc2nc(n1)NS(=O)(=O)c1cccc(c1)C(=O)N(Cc1ncc(N3CC(C)(C)C3)cn1)[C@H](CCC1CC1)CO2] | | | | | | +++ |
| 1312 | 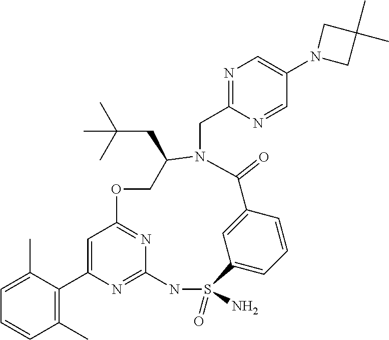 | | | | | | ++ |
| 1313 | 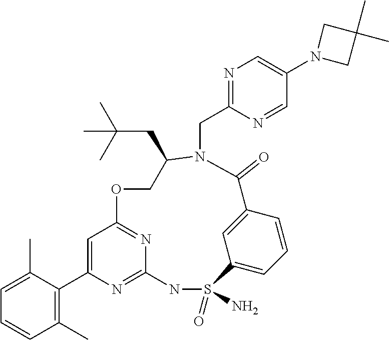 | | | | | | +++ |
| 1596 | 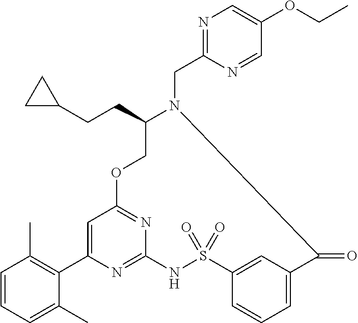 [SMILES: CCOc1cnc(CN2C(=O)c3cccc(c3)S(=O)(=O)Nc3nc(cc(-c4c(C)cccc4C)n3)OC[C@H]2CCC2CC2)nc1] | | | | | | +++ |
| 1597 | 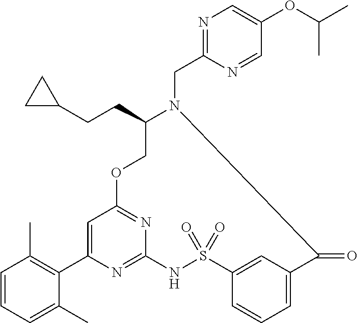 [SMILES: Cc1cccc(C)c1-c1cc2nc(n1)NS(=O)(=O)c1cccc(c1)C(=O)N(Cc1ncc(OC(C)C)cn1)[C@H](CCC1CC1)CO2] | | | | | | +++ |
| 1598 | 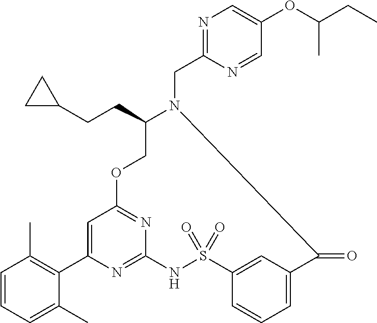 [SMILES: CCC(C)Oc1cnc(CN2C(=O)c3cccc(c3)S(=O)(=O)Nc3nc(cc(-c4c(C)cccc4C)n3)OC[C@H]2CCC2CC2)nc1] | | | | | | +++ |
| 1599 | 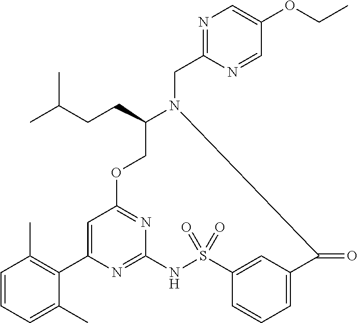 [SMILES: CCOc1cnc(CN2C(=O)c3cccc(c3)S(=O)(=O)Nc3nc(cc(-c4c(C)cccc4C)n3)OC[C@H]2CCC(C)C)nc1] | | | | | | +++ |
| 1600 | 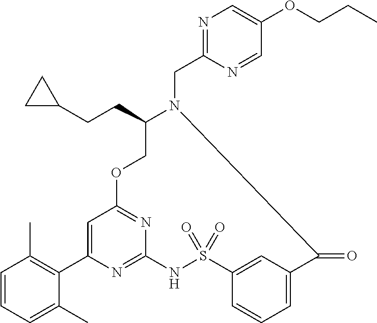 [SMILES: CCCOc1cnc(CN2C(=O)c3cccc(c3)S(=O)(=O)Nc3nc(cc(-c4c(C)cccc4C)n3)OC[C@H]2CCC2CC2)nc1] | | | | | | +++ |
| 1601 | 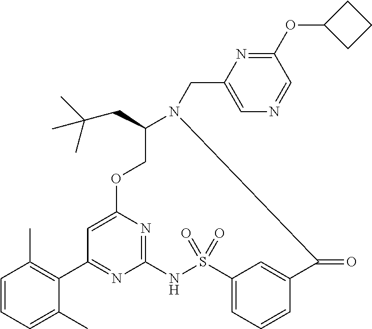 [SMILES: Cc1cccc(C)c1-c1cc2nc(n1)NS(=O)(=O)c1cccc(c1)C(=O)N(Cc1cncc(OC3CCC3)n1)[C@H](CC(C)(C)C)CO2] | | | | | | +++ |
| 1602 | 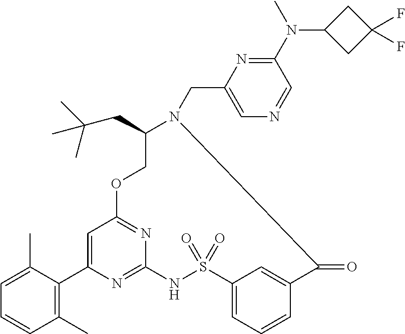 [SMILES: Cc1cccc(C)c1-c1cc2nc(n1)NS(=O)(=O)c1cccc(c1)C(=O)N(Cc1cncc(N(C)C3CC(F)(F)C3)n1)[C@H](CC(C)(C)C)CO2] | | | | +++ | +++ | |
| 1603 | 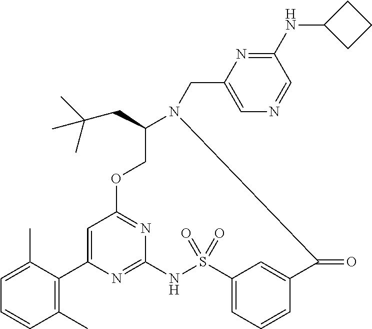 [SMILES: Cc1cccc(C)c1-c1cc2nc(n1)NS(=O)(=O)c1cccc(c1)C(=O)N(Cc1cncc(NC3CCC3)n1)[C@H](CC(C)(C)C)CO2] | | | | +++ | +++ | |
| 1604 | 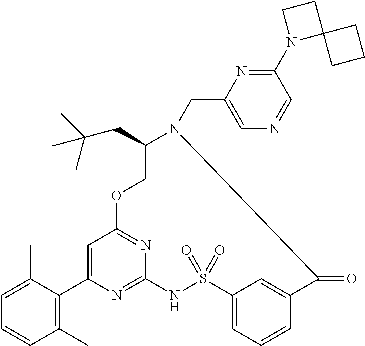 [SMILES: Cc1cccc(C)c1-c1cc2nc(n1)NS(=O)(=O)c1cccc(c1)C(=O)N(Cc1cncc(N3CCC34CCC4)n1)[C@H](CC(C)(C)C)CO2] | | | | +++ | +++ | |
| 1605 | 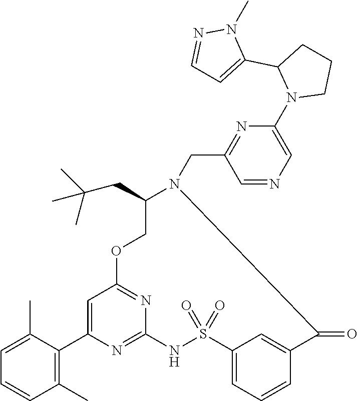 [SMILES: Cc1cccc(C)c1-c1cc2nc(n1)NS(=O)(=O)c1cccc(c1)C(=O)N(Cc1cncc(N3CCCC3c3ccnn3C)n1)[C@H](CC(C)(C)C)CO2] | | | | | | +++ |
| 1606 | 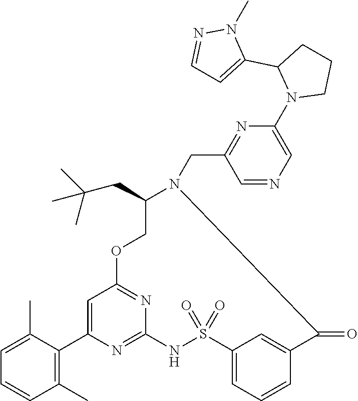 [SMILES: Cc1cccc(C)c1-c1cc2nc(n1)NS(=O)(=O)c1cccc(c1)C(=O)N(Cc1cncc(N3CCCC3c3ccnn3C)n1)[C@H](CC(C)(C)C)CO2] | | | | | | +++ |
| 1607 | 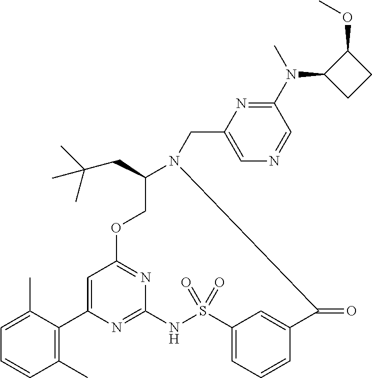 [SMILES: CO[C@H]1CC[C@H]1N(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1608 | 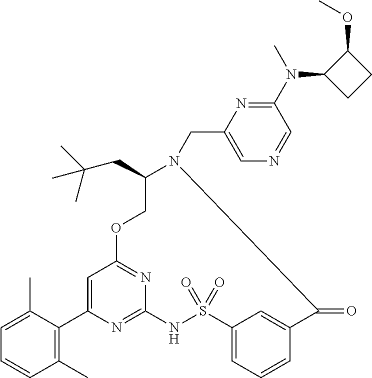 [SMILES: CO[C@H]1CC[C@H]1N(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1609 | 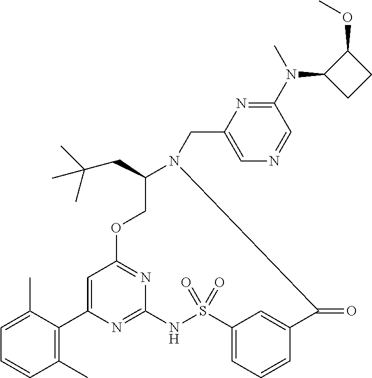 [SMILES: CO[C@H]1CC[C@H]1N(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1610 | 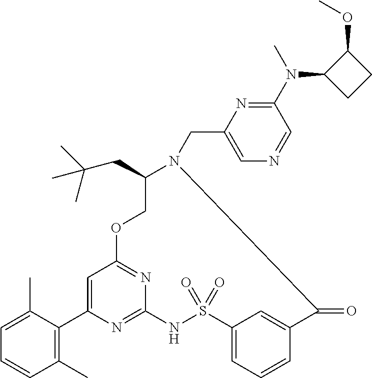 [SMILES: CO[C@H]1CC[C@H]1N(C)c1cncc(CN2C(=O)c3cccc(c3)S(=O)(=O)Nc3nc(cc(-c4c(C)cccc4C)n3)OC[C@H]2CC(C)(C)C)n1] | | | | +++ | +++ | +++ |
| 1611 | 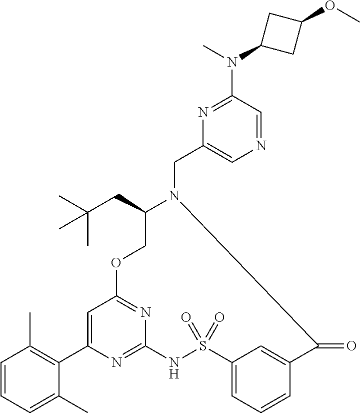 [SMILES: CO[C@H]1C[C@@H](N(C)c2cncc(CN3C(=O)c4cccc(c4)S(=O)(=O)Nc4nc(cc(-c5c(C)cccc5C)n4)OC[C@H]3CC(C)(C)C)n2)C1] | | | | | | +++ |
| 1612 | 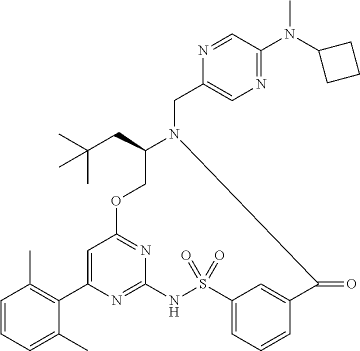 [SMILES: Cc1cccc(C)c1-c1cc2nc(n1)NS(=O)(=O)c1cccc(c1)C(=O)N(Cc1cnc(N(C)C3CCC3)cn1)[C@H](CC(C)(C)C)CO2] | | | | | | +++ |
| 1613 | 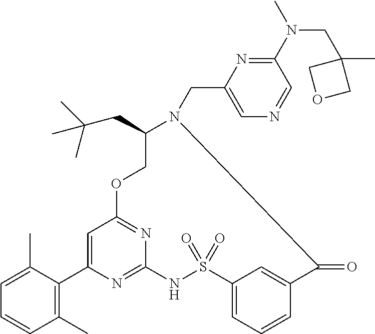 [SMILES: Cc1cccc(C)c1-c1cc2nc(n1)NS(=O)(=O)c1cccc(c1)C(=O)N(Cc1cncc(N(C)CC3(C)COC3)n1)[C@H](CC(C)(C)C)CO2] | | | | | | +++ |
| 1614 | 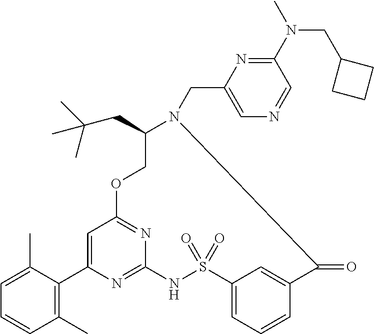 [SMILES: Cc1cccc(C)c1-c1cc2nc(n1)NS(=O)(=O)c1cccc(c1)C(=O)N(Cc1cncc(N(C)CC3CCC3)n1)[C@H](CC(C)(C)C)CO2] | | | | | | +++ |
| 1615 | 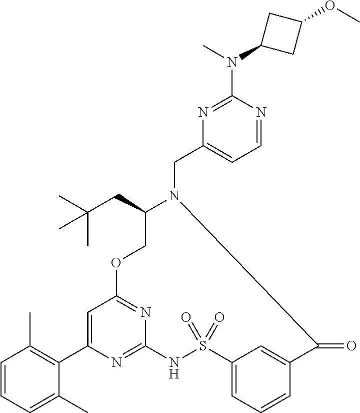 [SMILES: CO[C@H]1C[C@H](N(C)c2nccc(CN3C(=O)c4cccc(c4)S(=O)(=O)Nc4nc(cc(-c5c(C)cccc5C)n4)OC[C@H]3CC(C)(C)C)n2)C1] | | | | | | +++ |
| 1616 | 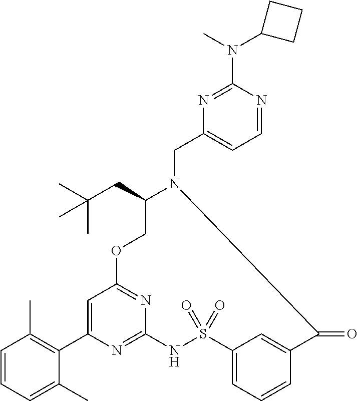 [SMILES: Cc1cccc(C)c1-c1cc2nc(n1)NS(=O)(=O)c1cccc(c1)C(=O)N(Cc1ccnc(N(C)C3CCC3)n1)[C@H](CC(C)(C)C)CO2] | | | | | | +++ |
| 1617 | 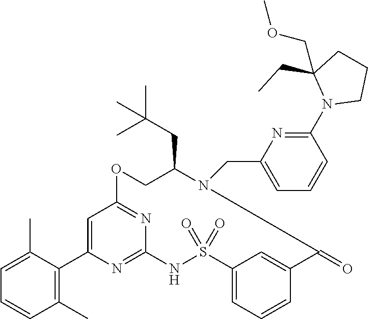 [SMILES: CC[C@@]1(COC)CCCN1c1cccc(CN2C(=O)c3cccc(c3)S(=O)(=O)Nc3nc(cc(-c4c(C)cccc4C)n3)OC[C@H]2CC(C)(C)C)n1] | | | | | | +++ |
| 1618 | 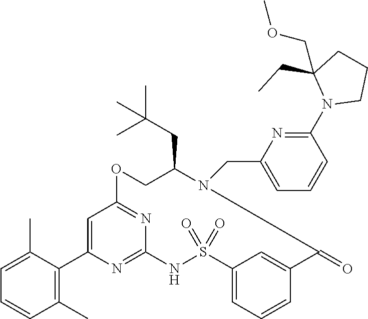 [SMILES: CC[C@@]1(COC)CCCN1c1cccc(CN2C(=O)c3cccc(c3)S(=O)(=O)Nc3nc(cc(-c4c(C)cccc4C)n3)OC[C@H]2CC(C)(C)C)n1] | | | | | | +++ |
| 1619 | 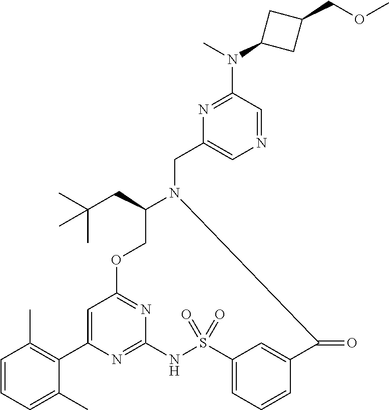 [SMILES: COC[C@H]1C[C@@H](N(C)c2cncc(CN3C(=O)c4cccc(c4)S(=O)(=O)Nc4nc(cc(-c5c(C)cccc5C)n4)OC[C@H]3CC(C)(C)C)n2)C1] | | | | | | +++ |
| 1620 | 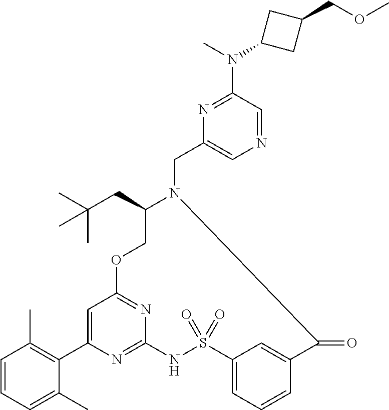 [SMILES: COC[C@H]1C[C@H](N(C)c2cncc(CN3C(=O)c4cccc(c4)S(=O)(=O)Nc4nc(cc(-c5c(C)cccc5C)n4)OC[C@H]3CC(C)(C)C)n2)C1] | | | | | | +++ |
| 1621 | 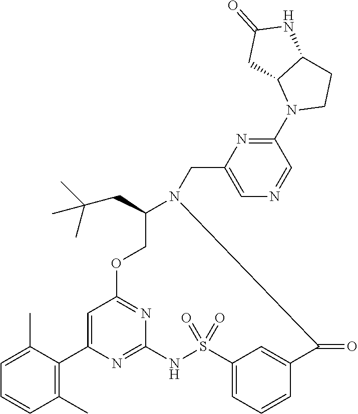 [SMILES: Cc1cccc(C)c1-c1cc2nc(n1)NS(=O)(=O)c1cccc(c1)C(=O)N(Cc1cncc(N3CC[C@H]4NC(=O)C[C@H]43)n1)[C@H](CC(C)(C)C)CO2] | | | | | | ++ |
| 1622 | 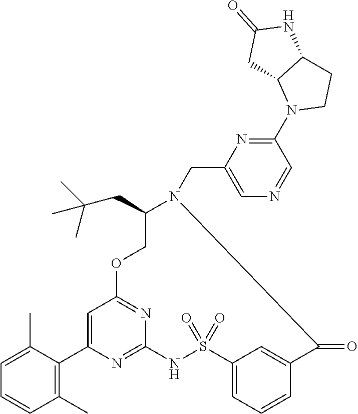 [SMILES: Cc1cccc(C)c1-c1cc2nc(n1)NS(=O)(=O)c1cccc(c1)C(=O)N(Cc1cncc(N3CC[C@H]4NC(=O)C[C@H]43)n1)[C@H](CC(C)(C)C)CO2] | | | | | | ++ |
| 1623 | 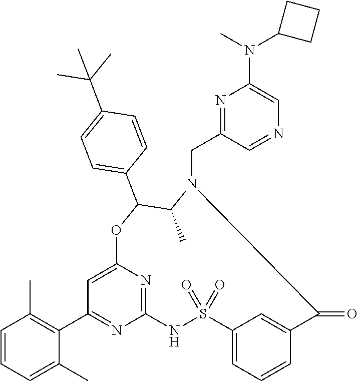 [SMILES: Cc1cccc(C)c1-c1cc2nc(n1)NS(=O)(=O)c1cccc(c1)C(=O)N(Cc1cncc(N(C)C3CCC3)n1)[C@H](C)C(c1ccc(C(C)(C)C)cc1)O2] | | | | | | ++ |
| 1314 | 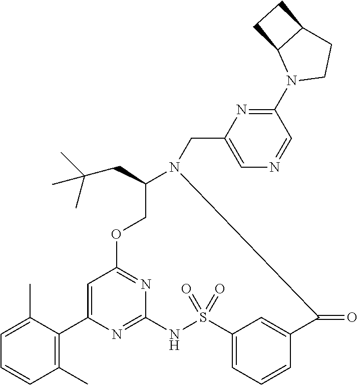 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](CC(C)(C)C)CO2] | | | | | | +++ |
| 1624 | 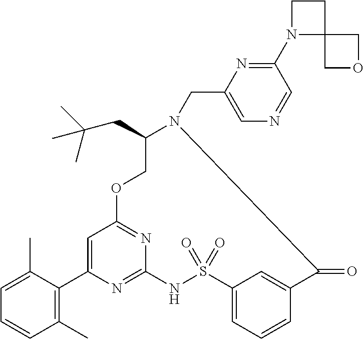 [SMILES: Cc1cccc(C)c1-c1cc2nc(n1)NS(=O)(=O)c1cccc(c1)C(=O)N(Cc1cncc(N3CCC34COC4)n1)[C@H](CC(C)(C)C)CO2] | | | | | | +++ |
| 1625 | 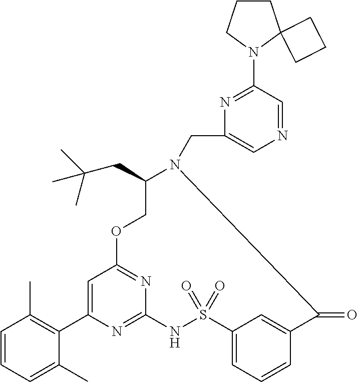 [SMILES: Cc1cccc(C)c1-c1cc2nc(n1)NS(=O)(=O)c1cccc(c1)C(=O)N(Cc1cncc(N3CCCC34CCC4)n1)[C@H](CC(C)(C)C)CO2] | | | | | | +++ |
| 1626 | 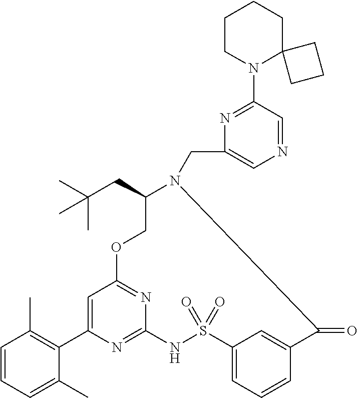 [SMILES: Cc1cccc(C)c1-c1cc2nc(n1)NS(=O)(=O)c1cccc(c1)C(=O)N(Cc1cncc(N3CCCCC34CCC4)n1)[C@H](CC(C)(C)C)CO2] | | | | | | +++ |
| 1627 | 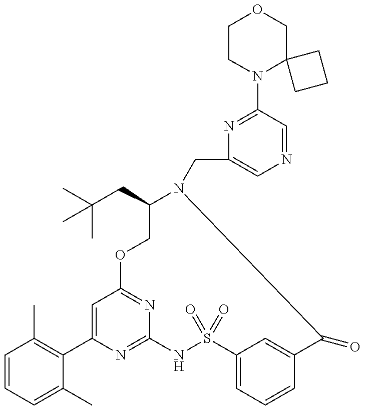 [SMILES: Cc1cccc(C)c1-c1cc2nc(n1)NS(=O)(=O)c1cccc(c1)C(=O)N(Cc1cncc(N3CCOCC34CCC4)n1)[C@H](CC(C)(C)C)CO2] | | | | | | +++ |
| 1316 | 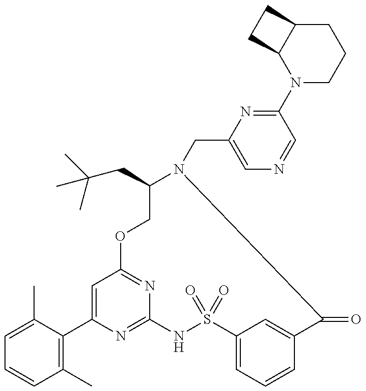 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CC(C)(C)C)CO2] | | | | | | +++ |
| 1315 | 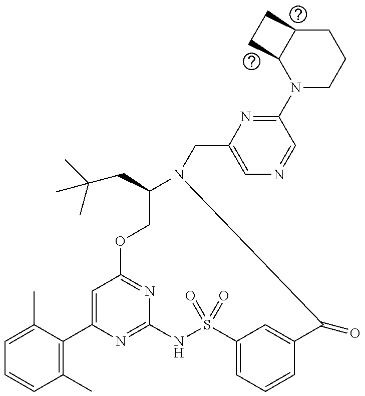 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CC(C)(C)C)CO2] | | | | +++ | +++ | +++ |
| 1628 | 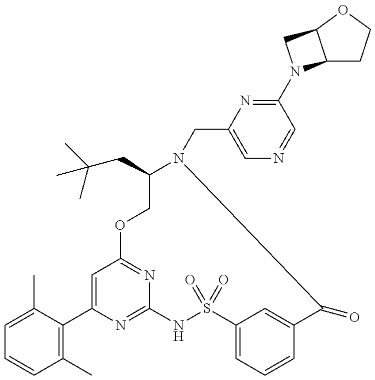 [SMILES: Cc1cccc(C)c1-c1cc2nc(n1)NS(=O)(=O)c1cccc(c1)C(=O)N(Cc1cncc(N3C[C@H]4OCC[C@H]43)n1)[C@H](CC(C)(C)C)CO2] | | | | | | +++ |
| 1629 | 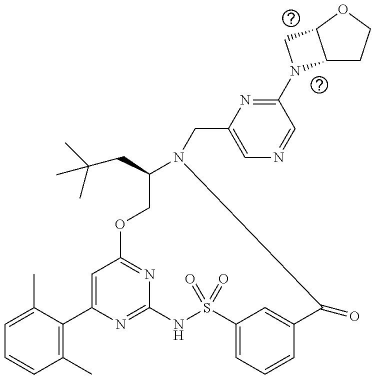 [SMILES: Cc1cccc(C)c1-c1cc2nc(n1)NS(=O)(=O)c1cccc(c1)C(=O)N(Cc1cncc(N3C[C@@H]4OCC[C@@H]43)n1)[C@H](CC(C)(C)C)CO2] | | | | | | +++ |
| 1630 | 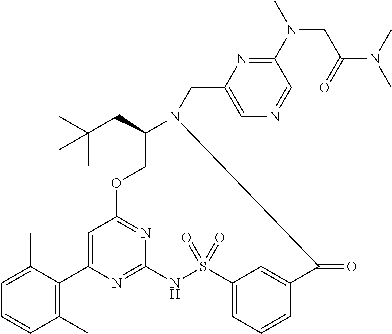 [SMILES: Cc1cccc(C)c1-c1cc2nc(n1)NS(=O)(=O)c1cccc(c1)C(=O)N(Cc1cncc(N(C)CC(=O)N(C)C)n1)[C@H](CC(C)(C)C)CO2] | | | | | | ++ |
| 1631 | 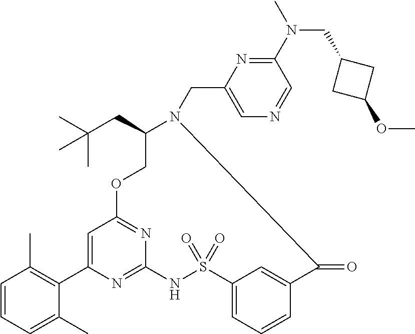 [SMILES: CO[C@H]1C[C@H](CN(C)c2cncc(CN3C(=O)c4cccc(c4)S(=O)(=O)Nc4nc(cc(-c5c(C)cccc5C)n4)OC[C@H]3CC(C)(C)C)n2)C1] | | | | | | +++ |
| 1632 | 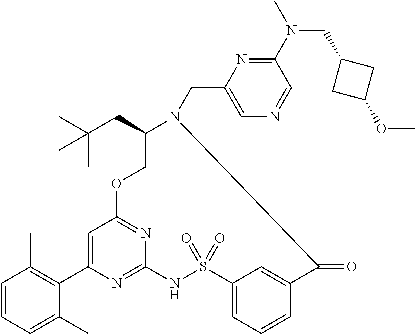 [SMILES: CO[C@H]1C[C@@H](CN(C)c2cncc(CN3C(=O)c4cccc(c4)S(=O)(=O)Nc4nc(cc(-c5c(C)cccc5C)n4)OC[C@H]3CC(C)(C)C)n2)C1] | | | | +++ | +++ | +++ |
| 1633 | 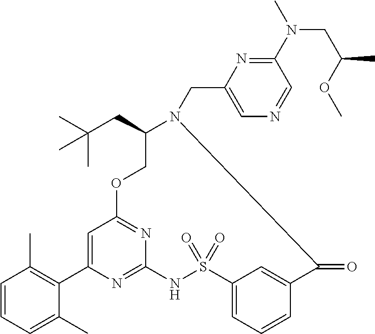 [SMILES: CO[C@H](C)CN(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1634 | 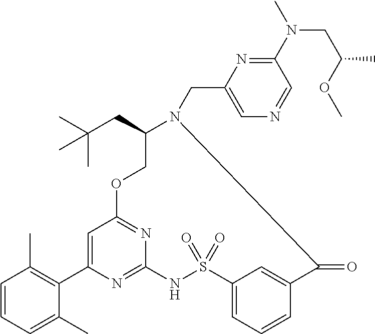 [SMILES: CO[C@@H](C)CN(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1635 | 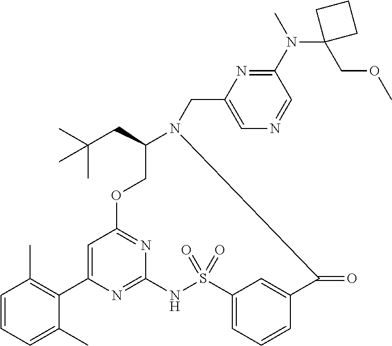 [SMILES: COCC1(N(C)c2cncc(CN3C(=O)c4cccc(c4)S(=O)(=O)Nc4nc(cc(-c5c(C)cccc5C)n4)OC[C@H]3CC(C)(C)C)n2)CCC1] | | | | | | +++ |
| 1636 | 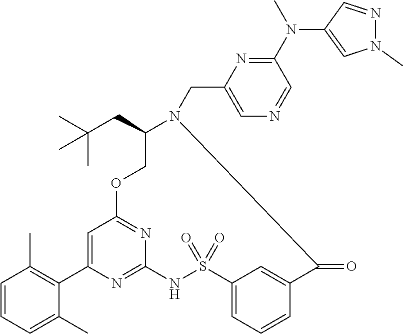 [SMILES: Cc1cccc(C)c1-c1cc2nc(n1)NS(=O)(=O)c1cccc(c1)C(=O)N(Cc1cncc(N(C)c3cnn(C)c3)n1)[C@H](CC(C)(C)C)CO2] | | | | | | +++ |
| 1637 | 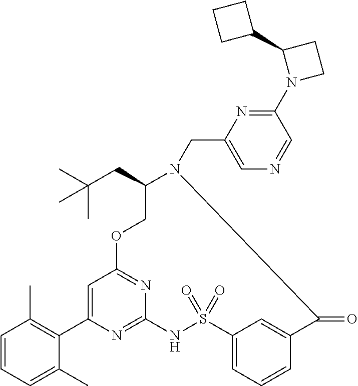 [SMILES: Cc1cccc(C)c1-c1cc2nc(n1)NS(=O)(=O)c1cccc(c1)C(=O)N(Cc1cncc(N3CC[C@@H]3C3CCC3)n1)[C@H](CC(C)(C)C)CO2] | | | | | | +++ |
| 1638 | 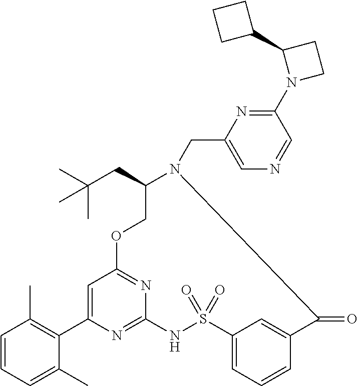 [SMILES: Cc1cccc(C)c1-c1cc2nc(n1)NS(=O)(=O)c1cccc(c1)C(=O)N(Cc1cncc(N3CC[C@@H]3C3CCC3)n1)[C@H](CC(C)(C)C)CO2] | | | | | | +++ |
| 1317 | 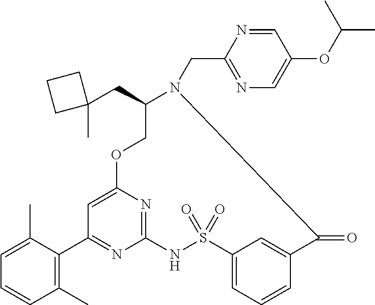 [SMILES: Cc1cccc(C)c1-c1cc2nc(n1)NS(=O)(=O)c1cccc(c1)C(=O)N(Cc1ncc(OC(C)C)cn1)[C@H](CC1(C)CCC1)CO2] | | | | | | +++ |
| 1639 | 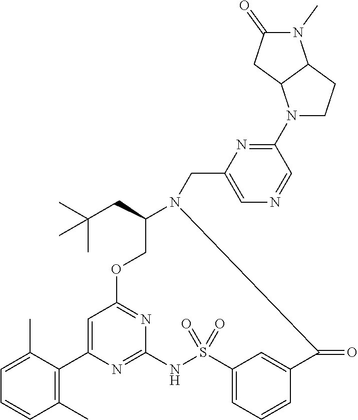 [SMILES: Cc1cccc(C)c1-c1cc2nc(n1)NS(=O)(=O)c1cccc(c1)C(=O)N(Cc1cncc(N3CCC4C3CC(=O)N4C)n1)[C@H](CC(C)(C)C)CO2] | | | | | | +++ |
| 1640 | 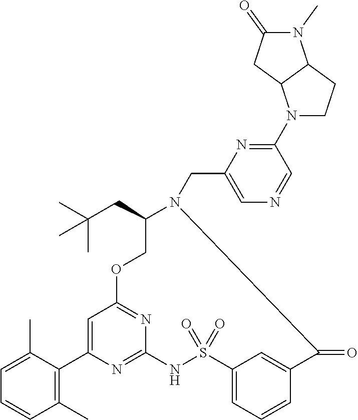 [SMILES: Cc1cccc(C)c1-c1cc2nc(n1)NS(=O)(=O)c1cccc(c1)C(=O)N(Cc1cncc(N3CCC4C3CC(=O)N4C)n1)[C@H](CC(C)(C)C)CO2] | | | | | | +++ |
| 1641 | 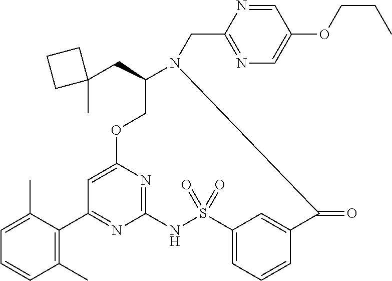 [SMILES: CCCOc1cnc(CN2C(=O)c3cccc(c3)S(=O)(=O)Nc3nc(cc(-c4c(C)cccc4C)n3)OC[C@H]2CC2(C)CCC2)nc1] | | | | | | +++ |
| 1642 | 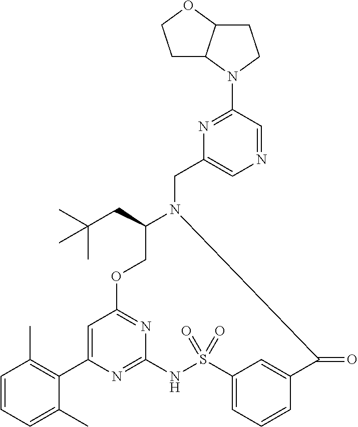 [SMILES: Cc1cccc(C)c1-c1cc2nc(n1)NS(=O)(=O)c1cccc(c1)C(=O)N(Cc1cncc(N3CCC4OCCC43)n1)[C@H](CC(C)(C)C)CO2] | | | | | | +++ |
| 1643 | 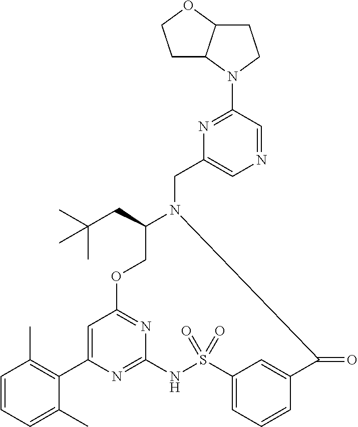 [SMILES: Cc1cccc(C)c1-c1cc2nc(n1)NS(=O)(=O)c1cccc(c1)C(=O)N(Cc1cncc(N3CCC4OCCC43)n1)[C@H](CC(C)(C)C)CO2] | | | | | | +++ |
| 1644 | 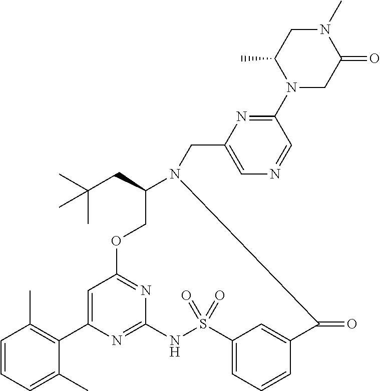 [SMILES: Cc1cccc(C)c1-c1cc2nc(n1)NS(=O)(=O)c1cccc(c1)C(=O)N(Cc1cncc(N3CC(=O)N(C)C[C@H]3C)n1)[C@H](CC(C)(C)C)CO2] | | | | | | +++ |
| 1318 | 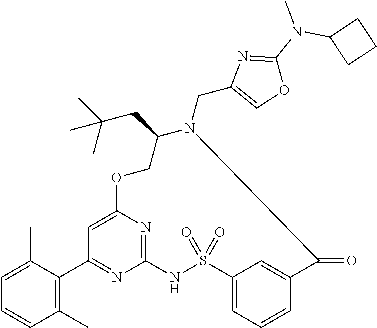 [SMILES: Cc1cccc(C)c1-c1cc2nc(n1)NS(=O)(=O)c1cccc(c1)C(=O)N(Cc1coc(N(C)C3CCC3)n1)[C@H](CC(C)(C)C)CO2] | | | | | | +++ |
| 1645 | 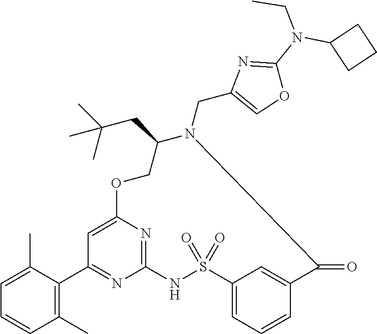 [SMILES: CCN(c1nc(CN2C(=O)c3cccc(c3)S(=O)(=O)Nc3nc(cc(-c4c(C)cccc4C)n3)OC[C@H]2CC(C)(C)C)co1)C1CCC1] | | | | | | +++ |
| 1646 | 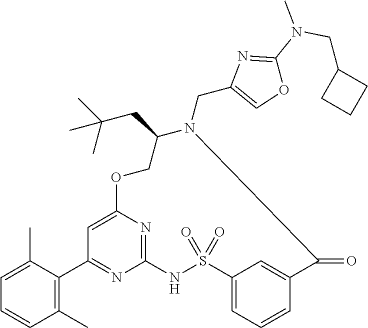 [SMILES: Cc1cccc(C)c1-c1cc2nc(n1)NS(=O)(=O)c1cccc(c1)C(=O)N(Cc1coc(N(C)CC3CCC3)n1)[C@H](CC(C)(C)C)CO2] | | | | | | +++ |
| 1647 | 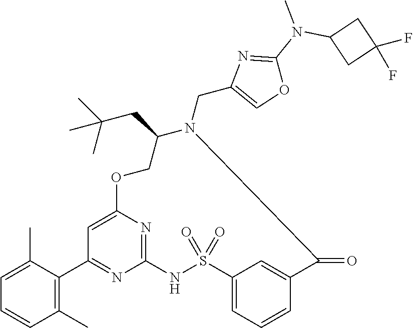 [SMILES: Cc1cccc(C)c1-c1cc2nc(n1)NS(=O)(=O)c1cccc(c1)C(=O)N(Cc1coc(N(C)C3CC(F)(F)C3)n1)[C@H](CC(C)(C)C)CO2] | | | | | | +++ |
| 1648 | 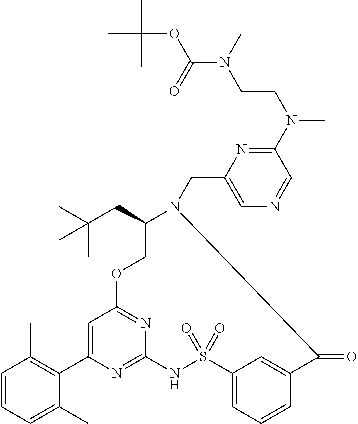 [SMILES: Cc1cccc(C)c1-c1cc2nc(n1)NS(=O)(=O)c1cccc(c1)C(=O)N(Cc1cncc(N(C)CCN(C)C(=O)OC(C)(C)C)n1)[C@H](CC(C)(C)C)CO2] | | | | | | +++ |
| 1649 | 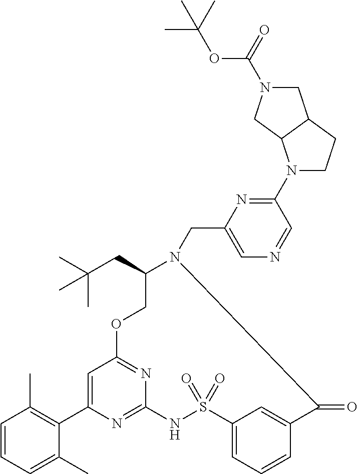 [SMILES: Cc1cccc(C)c1-c1cc2nc(n1)NS(=O)(=O)c1cccc(c1)C(=O)N(Cc1cncc(N3CCC4CN(C(=O)OC(C)(C)C)CC43)n1)[C@H](CC(C)(C)C)CO2] | | | | | | +++ |
| 1650 | 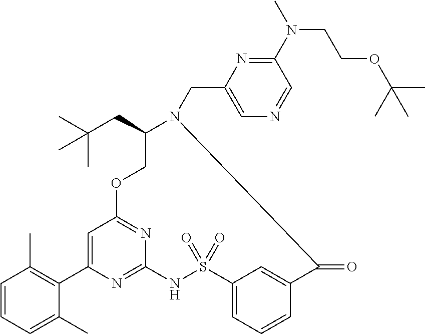 [SMILES: Cc1cccc(C)c1-c1cc2nc(n1)NS(=O)(=O)c1cccc(c1)C(=O)N(Cc1cncc(N(C)CCOC(C)(C)C)n1)[C@H](CC(C)(C)C)CO2] | | | | | | +++ |
| 1651 | 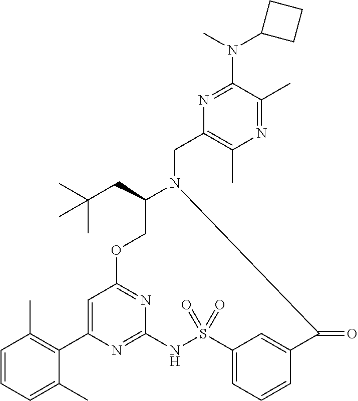 [SMILES: Cc1cccc(C)c1-c1cc2nc(n1)NS(=O)(=O)c1cccc(c1)C(=O)N(Cc1nc(N(C)C3CCC3)c(C)nc1C)[C@H](CC(C)(C)C)CO2] | | | | | | +++ |
| 1652 | 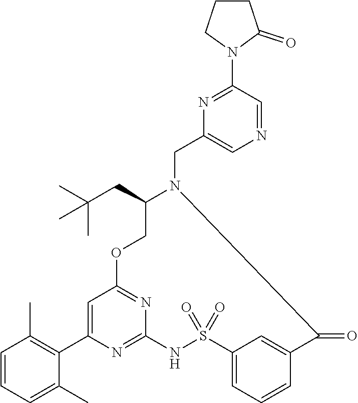 [SMILES: Cc1cccc(C)c1-c1cc2nc(n1)NS(=O)(=O)c1cccc(c1)C(=O)N(Cc1cncc(N3CCCC3=O)n1)[C@H](CC(C)(C)C)CO2] | | | | | | +++ |
| 1653 | 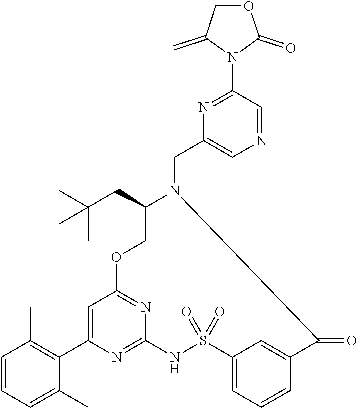 [SMILES: C=C1COC(=O)N1c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1654 | 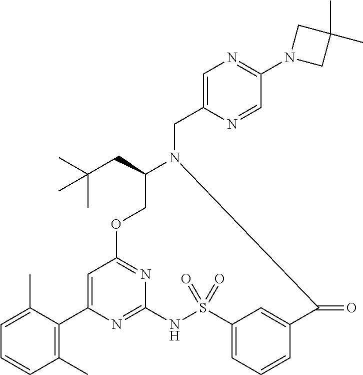 [SMILES: Cc1cccc(C)c1-c1cc2nc(n1)NS(=O)(=O)c1cccc(c1)C(=O)N(Cc1cnc(N3CC(C)(C)C3)cn1)[C@H](CC(C)(C)C)CO2] | | | | | | +++ |
| 1655 | 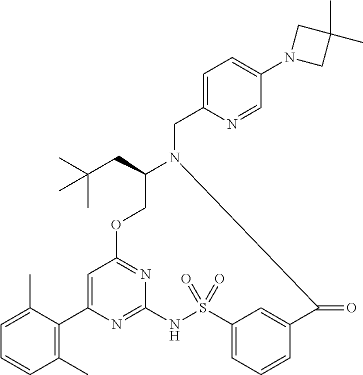 [SMILES: Cc1cccc(C)c1-c1cc2nc(n1)NS(=O)(=O)c1cccc(c1)C(=O)N(Cc1ccc(N3CC(C)(C)C3)cn1)[C@H](CC(C)(C)C)CO2] | | | | | | +++ |
| 1656 | 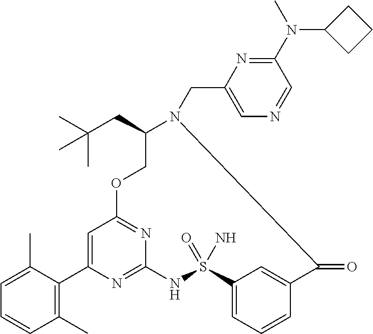 | | | | | | +++ |
| 1657 | 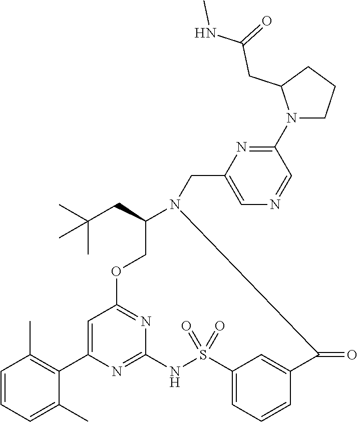 [SMILES: CNC(=O)CC1CCCN1c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1658 | 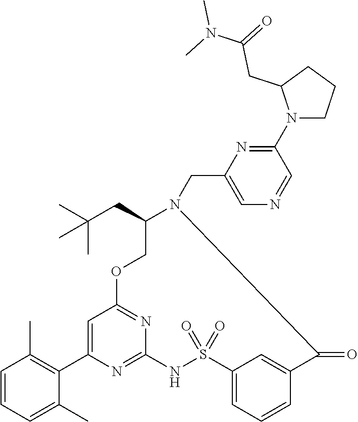 [SMILES: Cc1cccc(C)c1-c1cc2nc(n1)NS(=O)(=O)c1cccc(c1)C(=O)N(Cc1cncc(N3CCCC3CC(=O)N(C)C)n1)[C@H](CC(C)(C)C)CO2] | | | | | | +++ |
| 1659 | 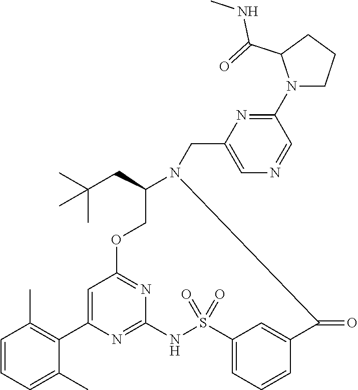 [SMILES: CNC(=O)C1CCCN1c1cncc(CN2C(=O)c3cccc(c3)S(=O)(=O)Nc3nc(cc(-c4c(C)cccc4C)n3)OC[C@H]2CC(C)(C)C)n1] | | | | | | |
| 1660 | 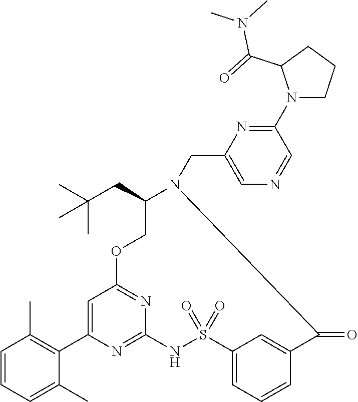 [SMILES: Cc1cccc(C)c1-c1cc2nc(n1)NS(=O)(=O)c1cccc(c1)C(=O)N(Cc1cncc(N3CCCC3C(=O)N(C)C)n1)[C@H](CC(C)(C)C)CO2] | | | | | | +++ |
| 1661 | 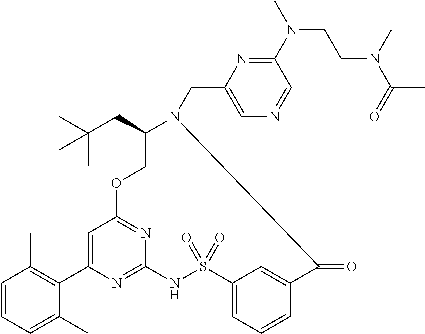 [SMILES: CC(=O)N(C)CCN(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1662 | 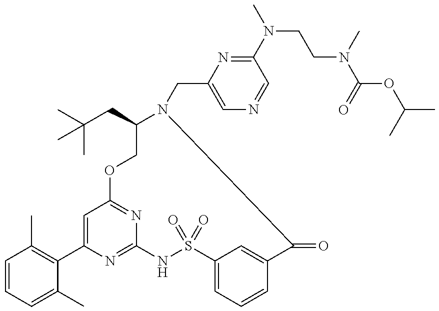 [SMILES: Cc1cccc(C)c1-c1cc2nc(n1)NS(=O)(=O)c1cccc(c1)C(=O)N(Cc1cncc(N(C)CCN(C)C(=O)OC(C)C)n1)[C@H](CC(C)(C)C)CO2] | | | | | | +++ |
| 1663 | 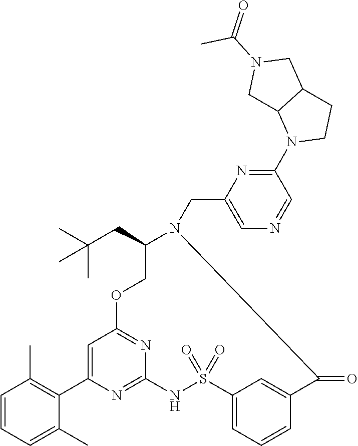 [SMILES: CC(=O)N1CC2CCN(c3cncc(CN4C(=O)c5cccc(c5)S(=O)(=O)Nc5nc(cc(-c6c(C)cccc6C)n5)OC[C@H]4CC(C)(C)C)n3)C2C1] | | | | | | +++ |
| 1664 | 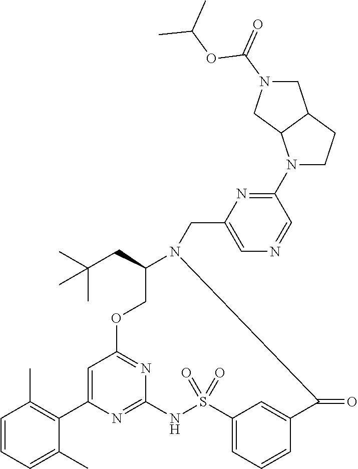 [SMILES: Cc1cccc(C)c1-c1cc2nc(n1)NS(=O)(=O)c1cccc(c1)C(=O)N(Cc1cncc(N3CCC4CN(C(=O)OC(C)C)CC43)n1)[C@H](CC(C)(C)C)CO2] | | | | | | +++ |
| 1665 | 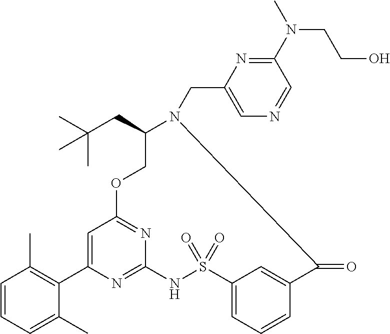 [SMILES: Cc1cccc(C)c1-c1cc2nc(n1)NS(=O)(=O)c1cccc(c1)C(=O)N(Cc1cncc(N(C)CCO)n1)[C@H](CC(C)(C)C)CO2] | | | | | | +++ |
| 1666 | 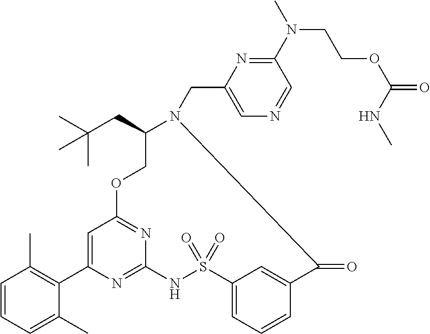 [SMILES: CNC(=O)OCCN(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1667 | 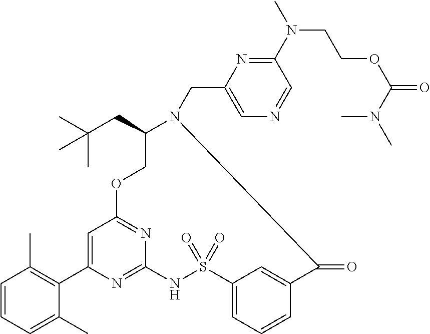 [SMILES: Cc1cccc(C)c1-c1cc2nc(n1)NS(=O)(=O)c1cccc(c1)C(=O)N(Cc1cncc(N(C)CCOC(=O)N(C)C)n1)[C@H](CC(C)(C)C)CO2] | | | | | | +++ |
| 1668 | 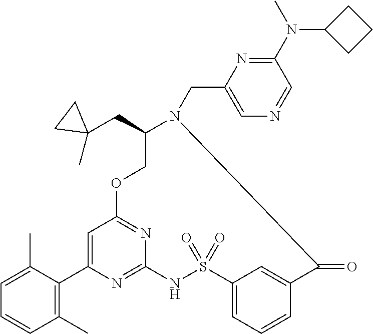 [SMILES: Cc1cccc(C)c1-c1cc2nc(n1)NS(=O)(=O)c1cccc(c1)C(=O)N(Cc1cncc(N(C)C3CCC3)n1)[C@H](CC1(C)CC1)CO2] | | | | +++ | +++ | +++ |
| 1669 | 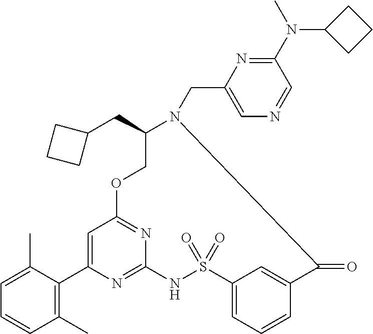 [SMILES: Cc1cccc(C)c1-c1cc2nc(n1)NS(=O)(=O)c1cccc(c1)C(=O)N(Cc1cncc(N(C)C3CCC3)n1)[C@H](CC1CCC1)CO2] | | | | +++ | +++ | +++ |
| 1670 | 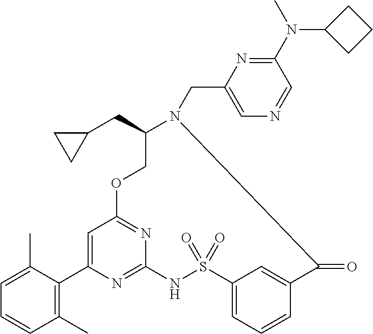 [SMILES: Cc1cccc(C)c1-c1cc2nc(n1)NS(=O)(=O)c1cccc(c1)C(=O)N(Cc1cncc(N(C)C3CCC3)n1)[C@H](CC1CC1)CO2] | | | | +++ | +++ | +++ |
| 1671 | 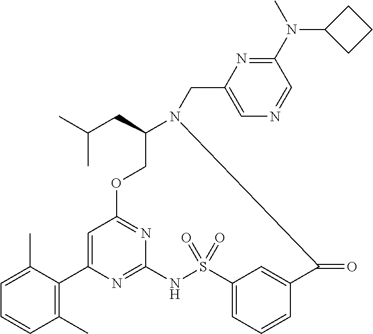 [SMILES: Cc1cccc(C)c1-c1cc2nc(n1)NS(=O)(=O)c1cccc(c1)C(=O)N(Cc1cncc(N(C)C3CCC3)n1)[C@H](CC(C)C)CO2] | | | | +++ | +++ | +++ |
| 1672 | 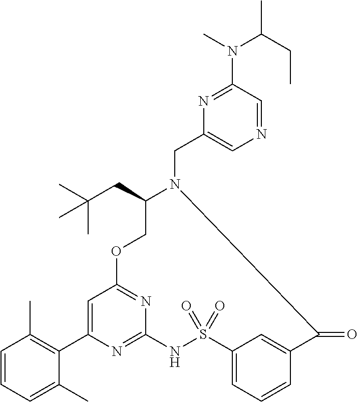 [SMILES: CCC(C)N(C)c1cncc(CN2C(=O)c3cccc(c3)S(=O)(=O)Nc3nc(cc(-c4c(C)cccc4C)n3)OC[C@H]2CC(C)(C)C)n1] | | | | +++ | +++ | +++ |
| 1673 | 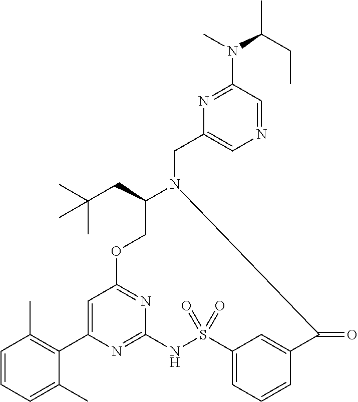 [SMILES: CC[C@H](C)N(C)c1cncc(CN2C(=O)c3cccc(c3)S(=O)(=O)Nc3nc(cc(-c4c(C)cccc4C)n3)OC[C@H]2CC(C)(C)C)n1] | | | | +++ | +++ | +++ |
| 1674 | 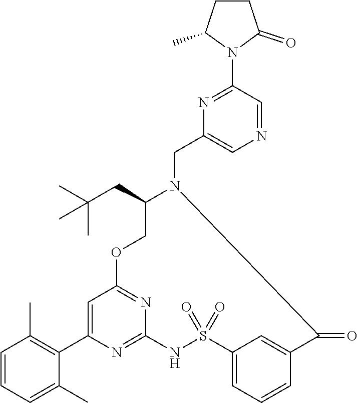 [SMILES: Cc1cccc(C)c1-c1cc2nc(n1)NS(=O)(=O)c1cccc(c1)C(=O)N(Cc1cncc(N3C(=O)CC[C@H]3C)n1)[C@H](CC(C)(C)C)CO2] | | | | | | +++ |
| 1675 | 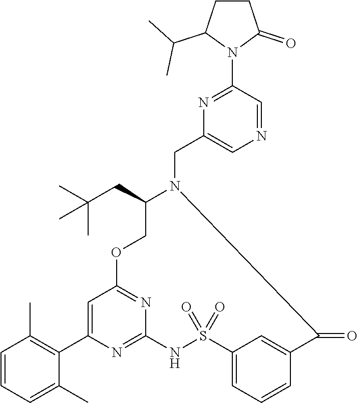 [SMILES: Cc1cccc(C)c1-c1cc2nc(n1)NS(=O)(=O)c1cccc(c1)C(=O)N(Cc1cncc(N3C(=O)CCC3C(C)C)n1)[C@H](CC(C)(C)C)CO2] | | | | | | +++ |
| 1676 | 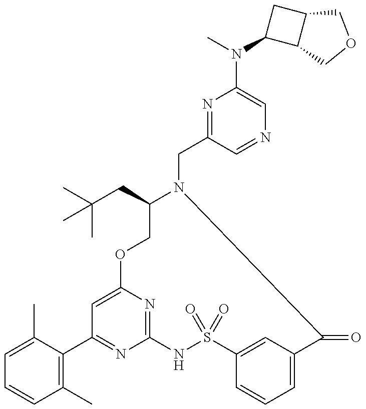 [SMILES: Cc1cccc(C)c1-c1cc2nc(n1)NS(=O)(=O)c1cccc(c1)C(=O)N(Cc1cncc(N(C)[C@H]3C[C@@H]4COC[C@@H]43)n1)[C@H](CC(C)(C)C)CO2] | | | | | | +++ |
| 1677 | 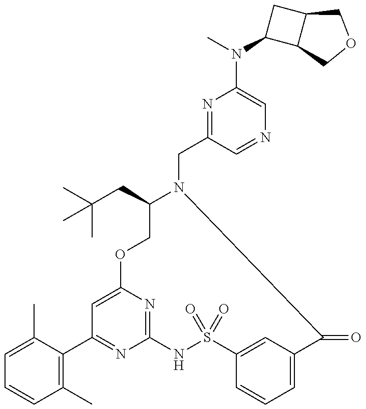 [SMILES: Cc1cccc(C)c1-c1cc2nc(n1)NS(=O)(=O)c1cccc(c1)C(=O)N(Cc1cncc(N(C)[C@H]3C[C@H]4COC[C@H]43)n1)[C@H](CC(C)(C)C)CO2] | | | | | | +++ |
| 1678 | 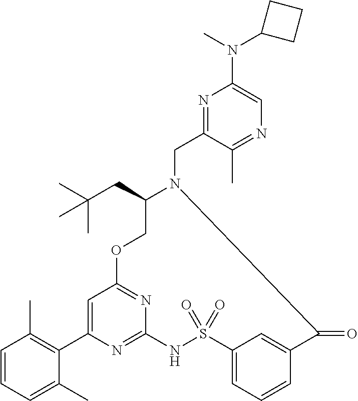 [SMILES: Cc1cccc(C)c1-c1cc2nc(n1)NS(=O)(=O)c1cccc(c1)C(=O)N(Cc1nc(N(C)C3CCC3)cnc1C)[C@H](CC(C)(C)C)CO2] | | | | | | +++ |
| 1679 | 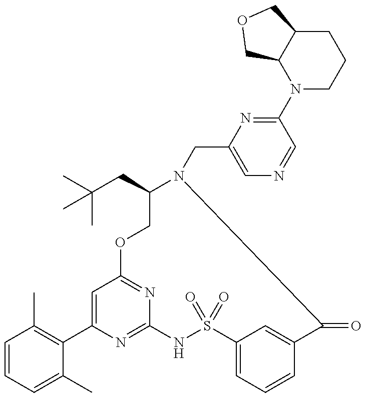 [SMILES: Cc1cccc(C)c1-c1cc2nc(n1)NS(=O)(=O)c1cccc(c1)C(=O)N(Cc1cncc(N3CCC[C@@H]4COC[C@@H]43)n1)[C@H](CC(C)(C)C)CO2] | | | | | | +++ |
| 1680 | 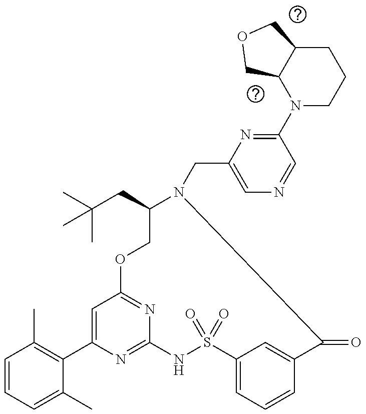 [SMILES: Cc1cccc(C)c1-c1cc2nc(n1)NS(=O)(=O)c1cccc(c1)C(=O)N(Cc1cncc(N3CCC[C@@H]4COC[C@@H]43)n1)[C@H](CC(C)(C)C)CO2] | | | | | | +++ |
| 1681 | 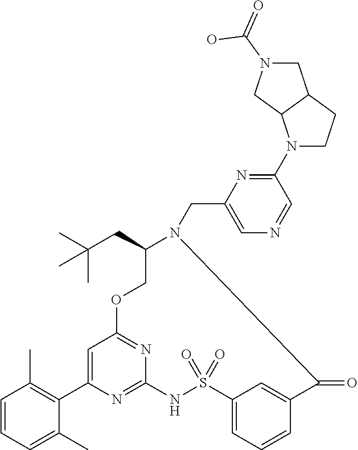 [SMILES: Cc1cccc(C)c1-c1cc2nc(n1)NS(=O)(=O)c1cccc(c1)C(=O)N(Cc1cncc(N3CCC4CN(C(=O)O)CC43)n1)[C@H](CC(C)(C)C)CO2] | | | | | | +++ |
| 1682 | 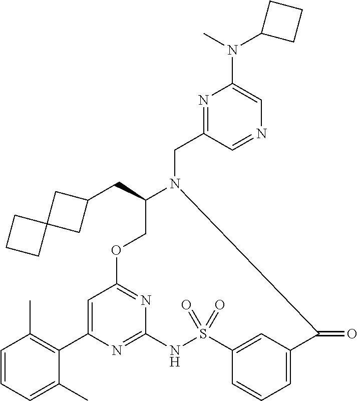 [SMILES: Cc1cccc(C)c1-c1cc2nc(n1)NS(=O)(=O)c1cccc(c1)C(=O)N(Cc1cncc(N(C)C3CCC3)n1)[C@H](CC1CC3(CCC3)C1)CO2] | | | | | | +++ |
| 1683 | 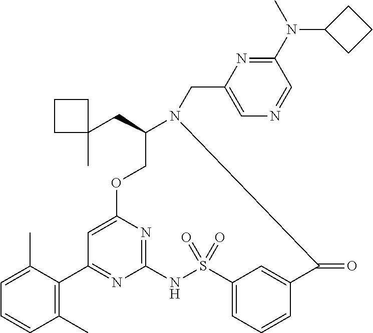 [SMILES: Cc1cccc(C)c1-c1cc2nc(n1)NS(=O)(=O)c1cccc(c1)C(=O)N(Cc1cncc(N(C)C3CCC3)n1)[C@H](CC1(C)CCC1)CO2] | | | | +++ | +++ | +++ |
| 1684 | 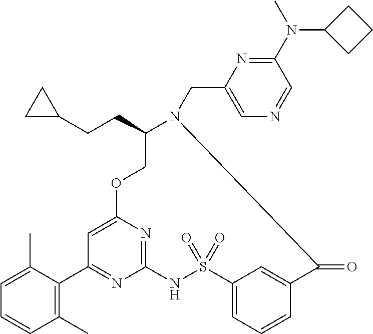 [SMILES: Cc1cccc(C)c1-c1cc2nc(n1)NS(=O)(=O)c1cccc(c1)C(=O)N(Cc1cncc(N(C)C3CCC3)n1)[C@H](CCC1CC1)CO2] | | | | +++ | +++ | +++ |
| 1319 | 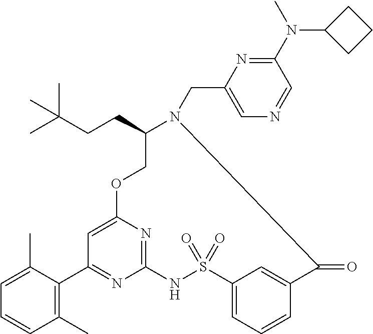 [SMILES: Cc1cccc(C)c1-c1cc2nc(n1)NS(=O)(=O)c1cccc(c1)C(=O)N(Cc1cncc(N(C)C3CCC3)n1)[C@H](CCC(C)(C)C)CO2] | | | | +++ | +++ | +++ |
| 1685 | 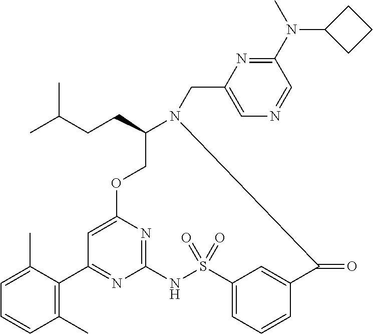 [SMILES: Cc1cccc(C)c1-c1cc2nc(n1)NS(=O)(=O)c1cccc(c1)C(=O)N(Cc1cncc(N(C)C3CCC3)n1)[C@H](CCC(C)C)CO2] | | | | +++ | +++ | +++ |
| 1686 | 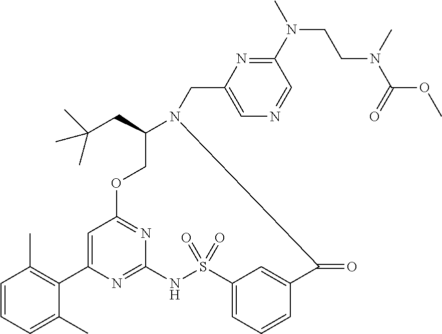 [SMILES: COC(=O)N(C)CCN(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1687 | 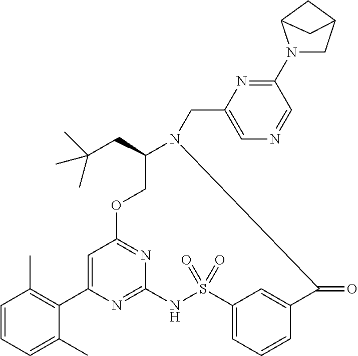 [SMILES: Cc1cccc(C)c1-c1cc2nc(n1)NS(=O)(=O)c1cccc(c1)C(=O)N(Cc1cncc(N3CC4CC3C4)n1)[C@H](CC(C)(C)C)CO2] | | | | | | +++ |
| 1688 | 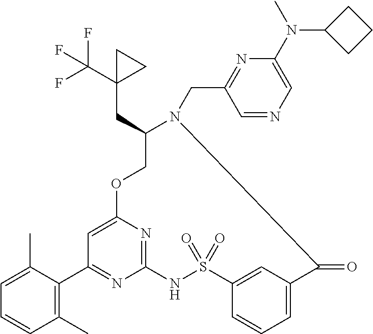 [SMILES: Cc1cccc(C)c1-c1cc2nc(n1)NS(=O)(=O)c1cccc(c1)C(=O)N(Cc1cncc(N(C)C3CCC3)n1)[C@H](CC1(C(F)(F)F)CC1)CO2] | | | | +++ | +++ | +++ |
| 1689 | 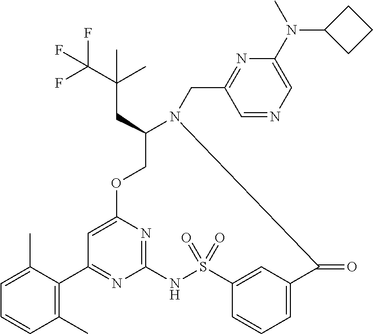 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)C(F)(F)F)CO2] | | | | +++ | +++ | +++ |
| 1690 | 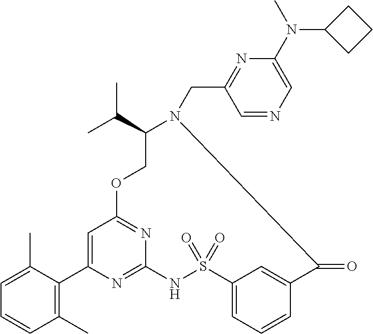 [SMILES: Cc1cccc(C)c1-c1cc2nc(n1)NS(=O)(=O)c1cccc(c1)C(=O)N(Cc1cncc(N(C)C3CCC3)n1)[C@H](C(C)C)CO2] | | | | | | +++ |
| 1691 | 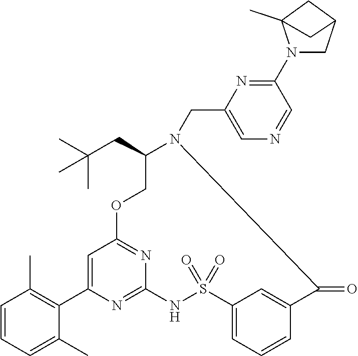 [SMILES: Cc1cccc(C)c1-c1cc2nc(n1)NS(=O)(=O)c1cccc(c1)C(=O)N(Cc1cncc(N3CC4CC3(C)C4)n1)[C@H](CC(C)(C)C)CO2] | | | | | | +++ |
| 1692 | 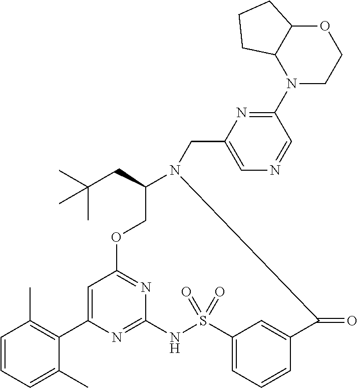 [SMILES: Cc1cccc(C)c1-c1cc2nc(n1)NS(=O)(=O)c1cccc(c1)C(=O)N(Cc1cncc(N3CCOC4CCCC43)n1)[C@H](CC(C)(C)C)CO2] | | | | | | +++ |
| 1693 | 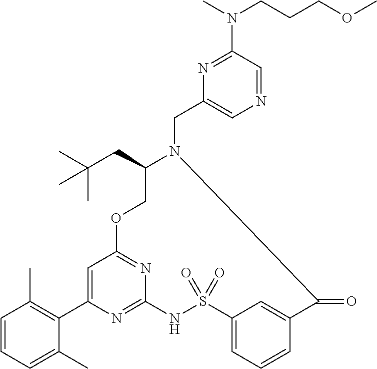 [SMILES: COCCCN(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1694 | 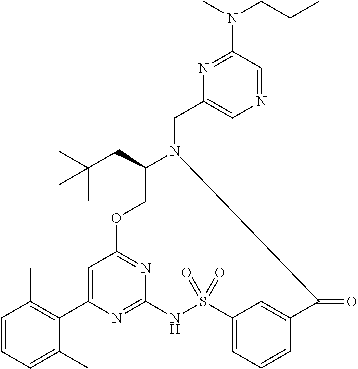 [SMILES: CCCN(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1605 | 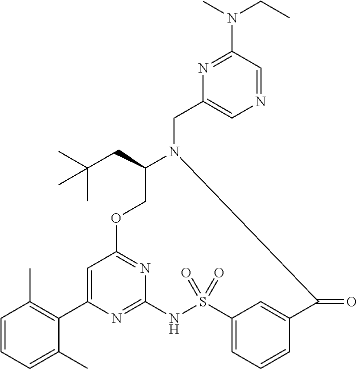 [SMILES: CCN(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1696 | 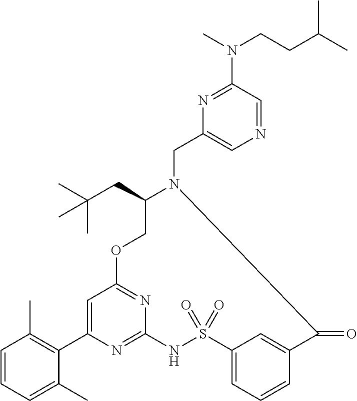 [SMILES: Cc1cccc(C)c1-c1cc2nc(n1)NS(=O)(=O)c1cccc(c1)C(=O)N(Cc1cncc(N(C)CCC(C)C)n1)[C@H](CC(C)(C)C)CO2] | | | | | | +++ |
| 1697 | 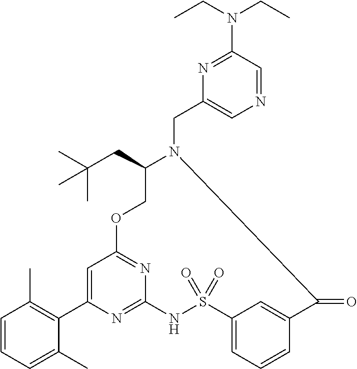 [SMILES: CCN(CC)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1320 | 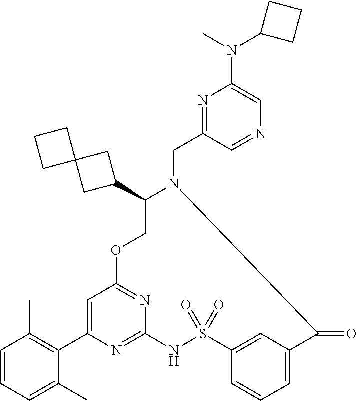 [SMILES: Cc1cccc(C)c1-c1cc2nc(n1)NS(=O)(=O)c1cccc(c1)C(=O)N(Cc1cncc(N(C)C3CCC3)n1)[C@H](C1CC3(CCC3)C1)CO2] | | | | +++ | +++ | +++ |
| 1321 | 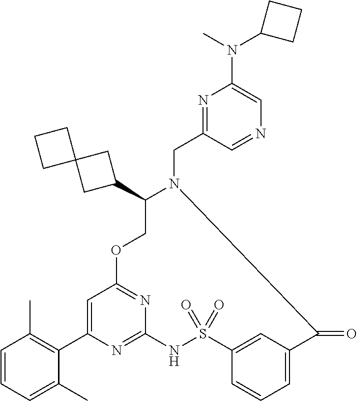 [SMILES: Cc1cccc(C)c1-c1cc2nc(n1)NS(=O)(=O)c1cccc(c1)C(=O)N(Cc1cncc(N(C)C3CCC3)n1)[C@H](C1CC3(CCC3)C1)CO2] | | | | | | ++ |
| 1698 | 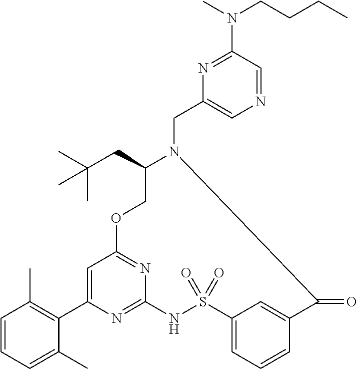 [SMILES: CCCCN(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1699 | 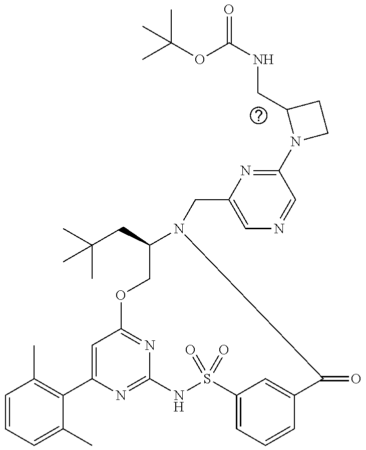 [SMILES: Cc1cccc(C)c1-c1cc2nc(n1)NS(=O)(=O)c1cccc(c1)C(=O)N(Cc1cncc(N3CCC3CNC(=O)OC(C)(C)C)n1)[C@H](CC(C)(C)C)CO2] | | | | | | +++ |
| 1700 | 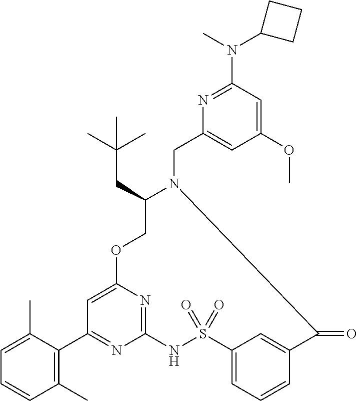 [SMILES: COc1cc(CN2C(=O)c3cccc(c3)S(=O)(=O)Nc3nc(cc(-c4c(C)cccc4C)n3)OC[C@H]2CC(C)(C)C)nc(N(C)C2CCC2)c1] | | | | | | +++ |
| 1701 | 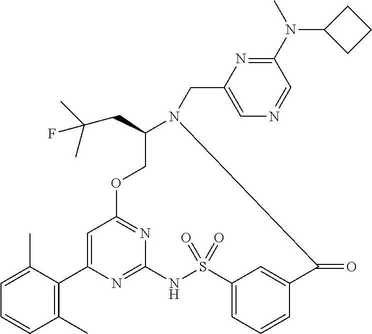 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)F)CO2] | | | | +++ | +++ | |
| 1702 | 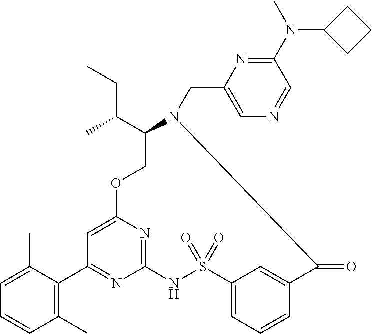 [SMILES: CC[C@@H](C)[C@@H]1COc2cc(-c3c(C)cccc3C)nc(n2)NS(=O)(=O)c2cccc(c2)C(=O)N1Cc1cncc(N(C)C2CCC2)n1] | | | | +++ | +++ | |
| 1703 | 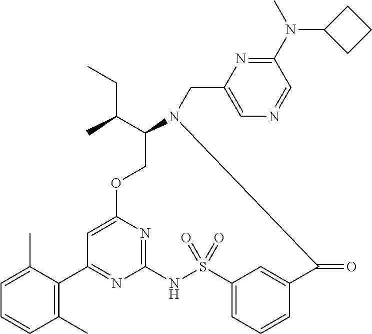 [SMILES: CC[C@H](C)[C@@H]1COc2cc(-c3c(C)cccc3C)nc(n2)NS(=O)(=O)c2cccc(c2)C(=O)N1Cc1cncc(N(C)C2CCC2)n1] | | | | +++ | +++ | |
| 1704 | 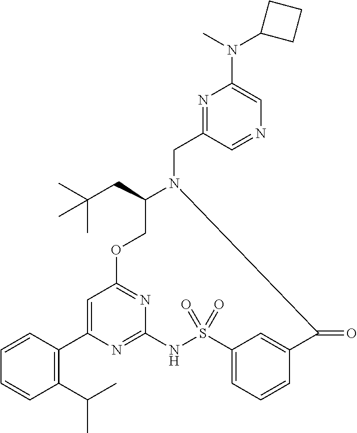 [SMILES: CC(C)c1ccccc1-c1cc2nc(n1)NS(=O)(=O)c1cccc(c1)C(=O)N(Cc1cncc(N(C)C3CCC3)n1)[C@H](CC(C)(C)C)CO2] | | | | | | +++ |
| 1705 | 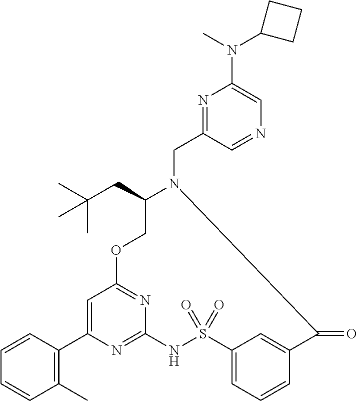 [SMILES: Cc1ccccc1-c1cc2nc(n1)NS(=O)(=O)c1cccc(c1)C(=O)N(Cc1cncc(N(C)C3CCC3)n1)[C@H](CC(C)(C)C)CO2] | | | | +++ | +++ | +++ |
| 1706 | 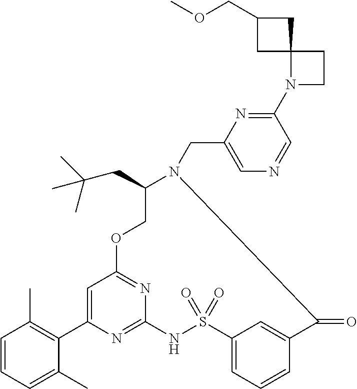 | | | | | | +++ |
| 1707 | 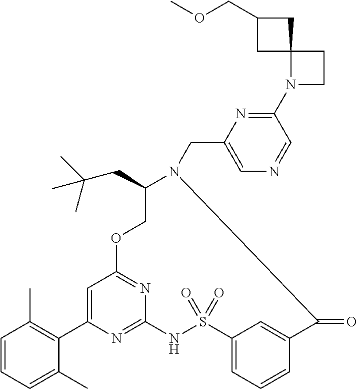 | | | | | | +++ |
| 1708 | 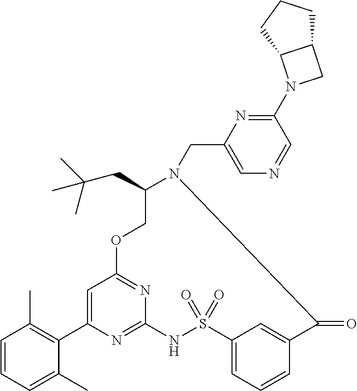 [SMILES: Cc1cccc(C)c1-c1cc2nc(n1)NS(=O)(=O)c1cccc(c1)C(=O)N(Cc1cncc(N3C[C@H]4CCC[C@H]43)n1)[C@H](CC(C)(C)C)CO2] | | | | | | +++ |
| 1709 | 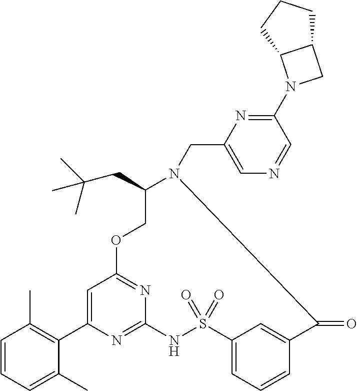 [SMILES: Cc1cccc(C)c1-c1cc2nc(n1)NS(=O)(=O)c1cccc(c1)C(=O)N(Cc1cncc(N3C[C@H]4CCC[C@H]43)n1)[C@H](CC(C)(C)C)CO2] | | | | | | +++ |
| 1710 | 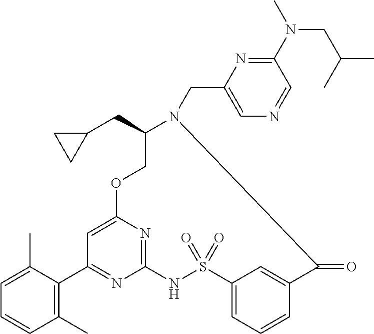 [SMILES: Cc1cccc(C)c1-c1cc2nc(n1)NS(=O)(=O)c1cccc(c1)C(=O)N(Cc1cncc(N(C)CC(C)C)n1)[C@H](CC1CC1)CO2] | | | | +++ | +++ | +++ |
| 1711 | 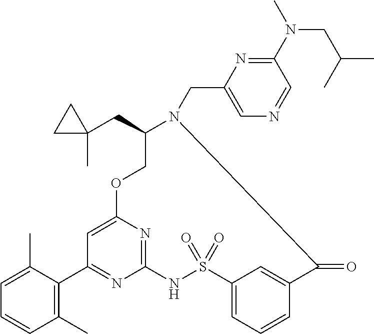 [SMILES: Cc1cccc(C)c1-c1cc2nc(n1)NS(=O)(=O)c1cccc(c1)C(=O)N(Cc1cncc(N(C)CC(C)C)n1)[C@H](CC1(C)CC1)CO2] | | | | +++ | +++ | +++ |
| 1712 | 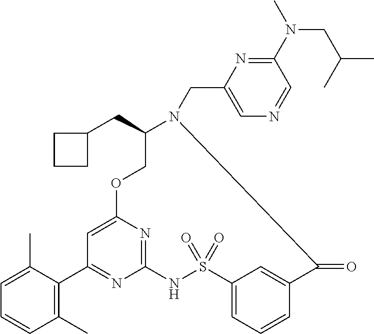 [SMILES: Cc1cccc(C)c1-c1cc2nc(n1)NS(=O)(=O)c1cccc(c1)C(=O)N(Cc1cncc(N(C)CC(C)C)n1)[C@H](CC1CCC1)CO2] | | | | +++ | +++ | +++ |
| 1713 | 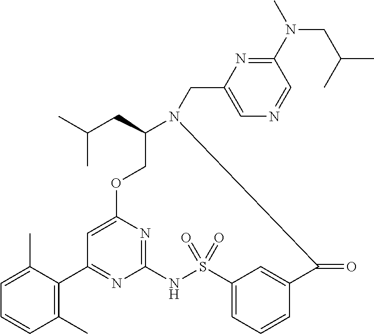 [SMILES: Cc1cccc(C)c1-c1cc2nc(n1)NS(=O)(=O)c1cccc(c1)C(=O)N(Cc1cncc(N(C)CC(C)C)n1)[C@H](CC(C)C)CO2] | | | | | | +++ |
| 1714 | 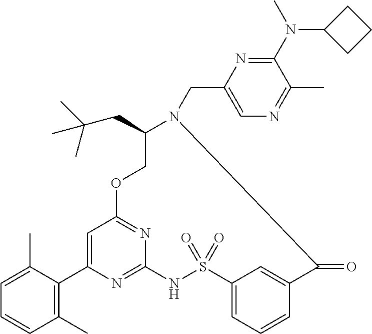 [SMILES: Cc1cccc(C)c1-c1cc2nc(n1)NS(=O)(=O)c1cccc(c1)C(=O)N(Cc1cnc(C)c(N(C)C3CCC3)n1)[C@H](CC(C)(C)C)CO2] | | | | | | +++ |
| 1322 | 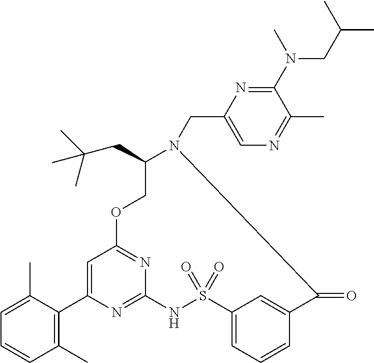 [SMILES: Cc1cccc(C)c1-c1cc2nc(n1)NS(=O)(=O)c1cccc(c1)C(=O)N(Cc1cnc(C)c(N(C)CC(C)C)n1)[C@H](CC(C)(C)C)CO2] | | | | | | +++ |
| 1715 | 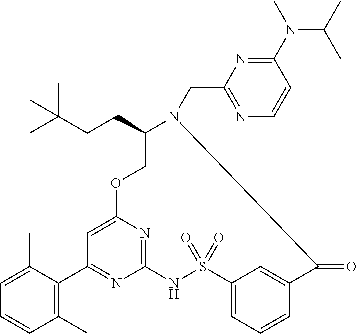 [SMILES: Cc1cccc(C)c1-c1cc2nc(n1)NS(=O)(=O)c1cccc(c1)C(=O)N(Cc1nccc(N(C)C(C)C)n1)[C@H](CCC(C)(C)C)CO2] | | | | | | +++ |
| 1716 | 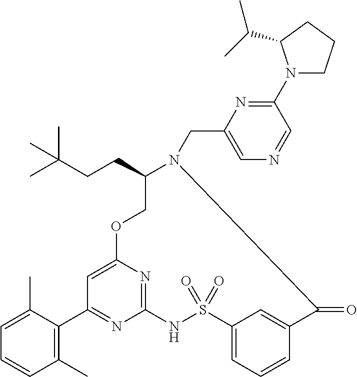 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](CCC(C)(C)C)CO2] | | | | +++ | +++ | +++ |
| 1717 | 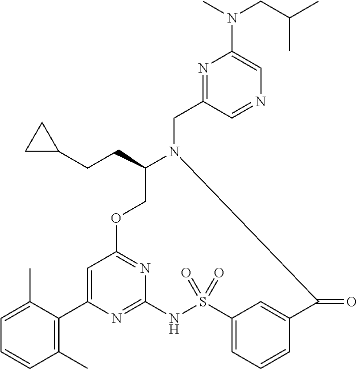 [SMILES: Cc1cccc(C)c1-c1cc2nc(n1)NS(=O)(=O)c1cccc(c1)C(=O)N(Cc1cncc(N(C)CC(C)C)n1)[C@H](CCC1CC1)CO2] | | | | | | +++ |
| 1718 | 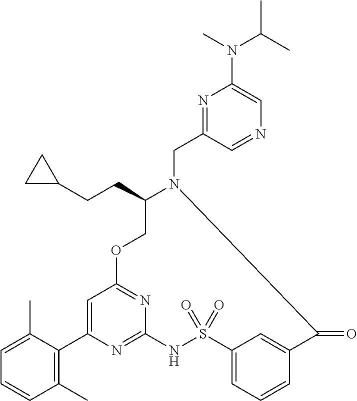 [SMILES: Cc1cccc(C)c1-c1cc2nc(n1)NS(=O)(=O)c1cccc(c1)C(=O)N(Cc1cncc(N(C)C(C)C)n1)[C@H](CCC1CC1)CO2] | | | | | | +++ |
| 1719 | 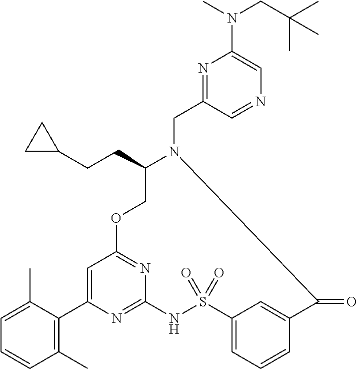 [SMILES: Cc1cccc(C)c1-c1cc2nc(n1)NS(=O)(=O)c1cccc(c1)C(=O)N(Cc1cncc(N(C)CC(C)(C)C)n1)[C@H](CCC1CC1)CO2] | | | | +++ | +++ | +++ |
| 1720 | 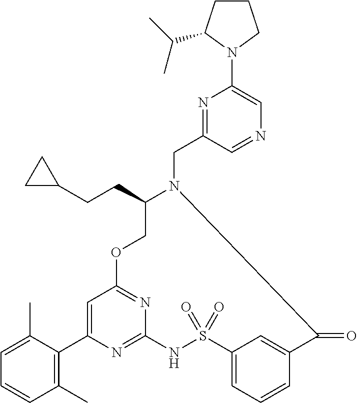 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](CCC1CC1)CO2] | | | | +++ | +++ | +++ |
| 1721 | 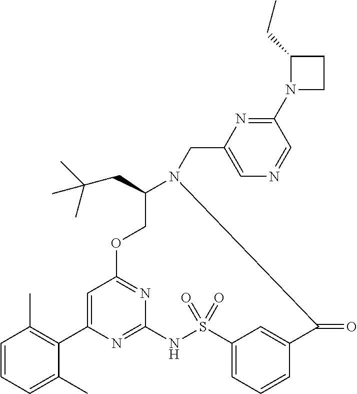 [SMILES: CC[C@@H]1CCN1c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1722 | 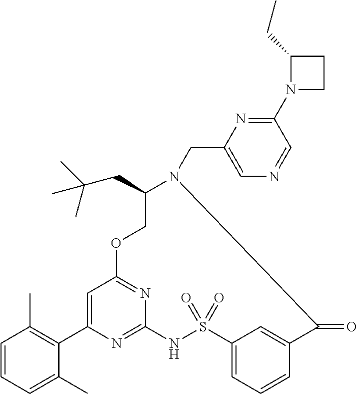 [SMILES: CC[C@@H]1CCN1c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1723 | 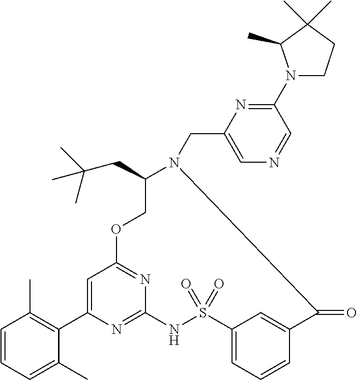 [SMILES: Cc1cccc(C)c1-c1cc2nc(n1)NS(=O)(=O)c1cccc(c1)C(=O)N(Cc1cncc(N3CCC(C)(C)[C@@H]3C)n1)[C@H](CC(C)(C)C)CO2] | | | | | | +++ |
| 1724 | 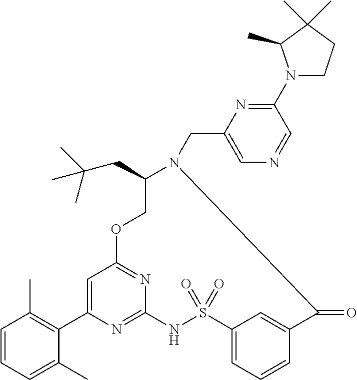 [SMILES: Cc1cccc(C)c1-c1cc2nc(n1)NS(=O)(=O)c1cccc(c1)C(=O)N(Cc1cncc(N3CCC(C)(C)[C@@H]3C)n1)[C@H](CC(C)(C)C)CO2] | | | | | | +++ |
| 1725 | 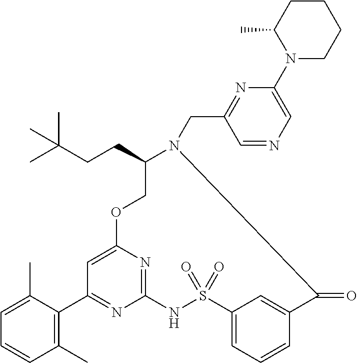 [SMILES: Cc1cccc(C)c1-c1cc2nc(n1)NS(=O)(=O)c1cccc(c1)C(=O)N(Cc1cncc(N3CCCC[C@H]3C)n1)[C@H](CCC(C)(C)C)CO2] | | | | +++ | +++ | +++ |
| 1726 | 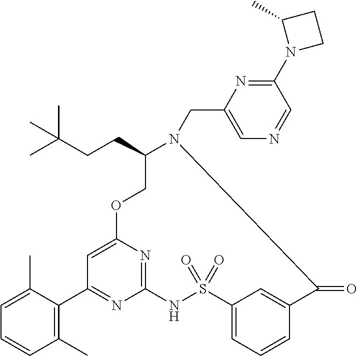 [SMILES: Cc1cccc(C)c1-c1cc2nc(n1)NS(=O)(=O)c1cccc(c1)C(=O)N(Cc1cncc(N3CC[C@H]3C)n1)[C@H](CCC(C)(C)C)CO2] | | | | +++ | +++ | +++ |
| 1727 | 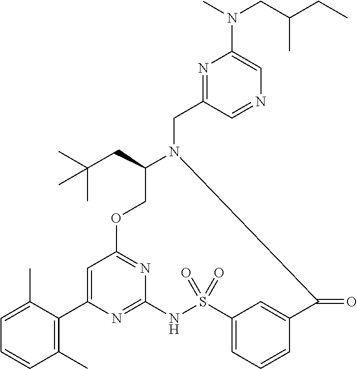 [SMILES: CCC(C)CN(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1728 | 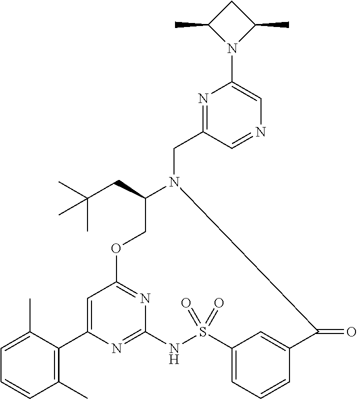 [SMILES: Cc1cccc(C)c1-c1cc2nc(n1)NS(=O)(=O)c1cccc(c1)C(=O)N(Cc1cncc(N3[C@H](C)C[C@@H]3C)n1)[C@H](CC(C)(C)C)CO2] | | | | | | +++ |
| 1729 | 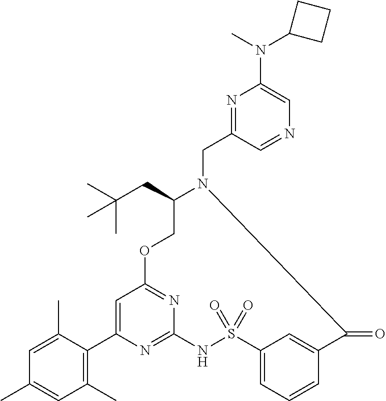 [SMILES: Cc1cc(C)c(-c2cc3nc(n2)NS(=O)(=O)c2cccc(c2)C(=O)N(Cc2cncc(N(C)C4CCC4)n2)[C@H](CC(C)(C)C)CO3)c(C)c1] | | | | | | +++ |
| 1730 | 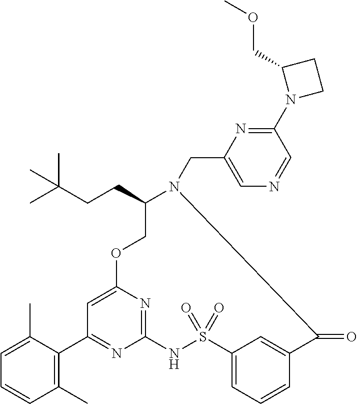 [SMILES: COC[C@@H]1CCN1c1cncc(CN2C(=O)c3cccc(c3)S(=O)(=O)Nc3nc(cc(-c4c(C)cccc4C)n3)OC[C@H]2CCC(C)(C)C)n1] | | | | +++ | +++ | +++ |
| 1731 | 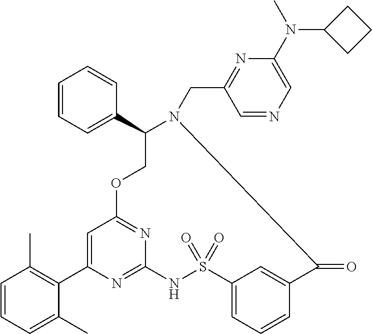 [SMILES: Cc1cccc(C)c1-c1cc2nc(n1)NS(=O)(=O)c1cccc(c1)C(=O)N(Cc1cncc(N(C)C3CCC3)n1)[C@H](c1ccccc1)CO2] | | | | | | +++ |
| 1732 | 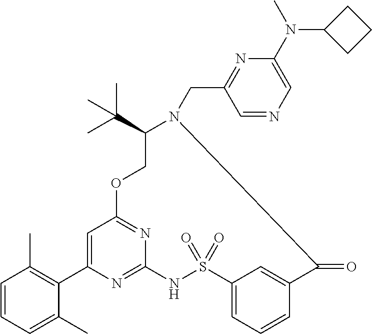 [SMILES: Cc1cccc(C)c1-c1cc2nc(n1)NS(=O)(=O)c1cccc(c1)C(=O)N(Cc1cncc(N(C)C3CCC3)n1)[C@H](C(C)(C)C)CO2] | | | | | | +++ |
| 1733 | 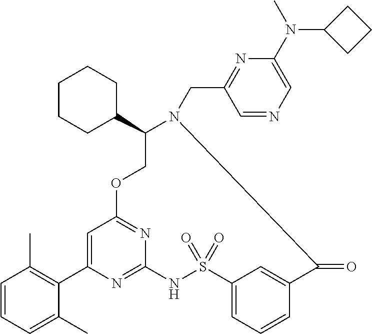 [SMILES: Cc1cccc(C)c1-c1cc2nc(n1)NS(=O)(=O)c1cccc(c1)C(=O)N(Cc1cncc(N(C)C3CCC3)n1)[C@H](C1CCCCC1)CO2] | | | | +++ | +++ | +++ |
| 1734 | 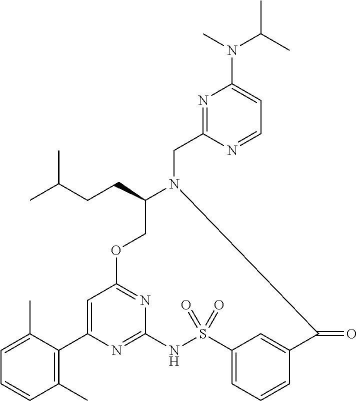 [SMILES: Cc1cccc(C)c1-c1cc2nc(n1)NS(=O)(=O)c1cccc(c1)C(=O)N(Cc1nccc(N(C)C(C)C)n1)[C@H](CCC(C)C)CO2] | | | | | | +++ |
| 1735 | 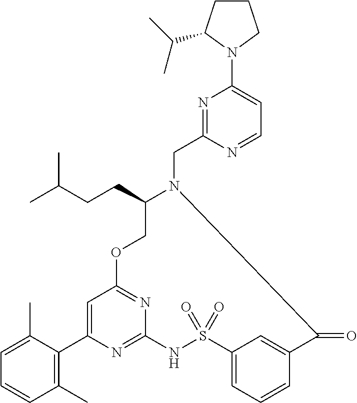 [SMILES: Cc1cccc(C)c1-c1cc2nc(n1)NS(=O)(=O)c1cccc(c1)C(=O)N(Cc1nccc(N3CCC[C@H]3C(C)C)n1)[C@H](CCC(C)C)CO2] | | | | ++ | +++ | +++ |
| 1736 | 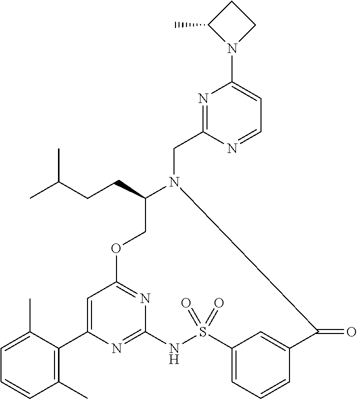 [SMILES: Cc1cccc(C)c1-c1cc2nc(n1)NS(=O)(=O)c1cccc(c1)C(=O)N(Cc1nccc(N3CC[C@H]3C)n1)[C@H](CCC(C)C)CO2] | | | | +++ | +++ | +++ |
| 1737 | 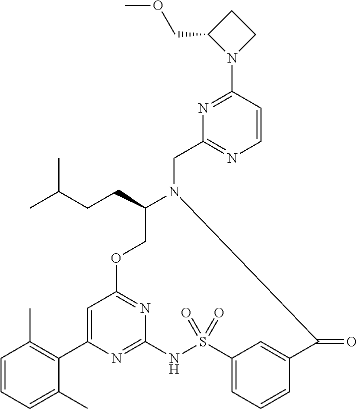 [SMILES: COC[C@@H]1CCN1c1ccnc(CN2C(=O)c3cccc(c3)S(=O)(=O)Nc3nc(cc(-c4c(C)cccc4C)n3)OC[C@H]2CCC(C)C)n1] | | | | +++ | +++ | +++ |
| 1738 | 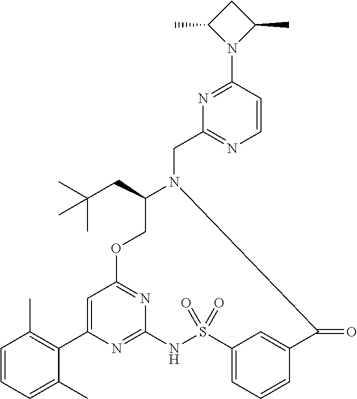 [SMILES: Cc1cccc(C)c1-c1cc2nc(n1)NS(=O)(=O)c1cccc(c1)C(=O)N(Cc1nccc(N3[C@H](C)C[C@H]3C)n1)[C@H](CC(C)(C)C)CO2] | | | | | | +++ |
| 1739 | 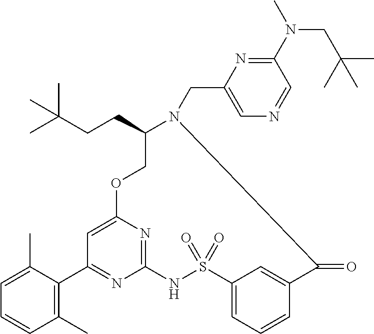 [SMILES: Cc1cccc(C)c1-c1cc2nc(n1)NS(=O)(=O)c1cccc(c1)C(=O)N(Cc1cncc(N(C)CC(C)(C)C)n1)[C@H](CCC(C)(C)C)CO2] | | | | | | +++ |
| 1740 | 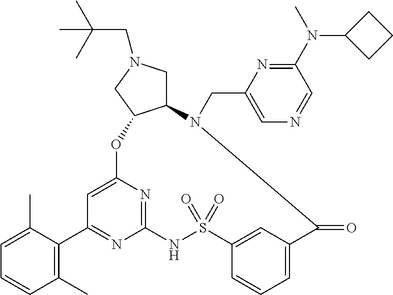 [SMILES: Cc1cccc(C)c1-c1cc2nc(n1)NS(=O)(=O)c1cccc(c1)C(=O)N(Cc1cncc(N(C)C3CCC3)n1)[C@@H]1CN(CC(C)(C)C)C[C@H]1O2] | | | | | | ++ |
| 1741 | 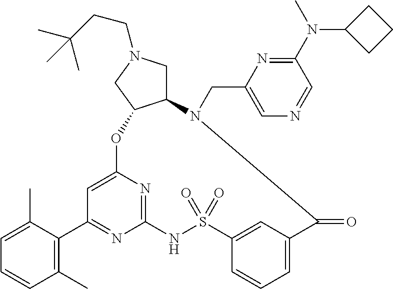 [SMILES: Cc1cccc(C)c1-c1cc2nc(n1)NS(=O)(=O)c1cccc(c1)C(=O)N(Cc1cncc(N(C)C3CCC3)n1)[C@@H]1CN(CCC(C)(C)C)C[C@H]1O2] | | | | | | ++ |
| 1742 | 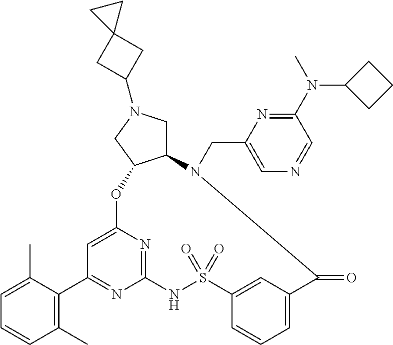 [SMILES: Cc1cccc(C)c1-c1cc2nc(n1)NS(=O)(=O)c1cccc(c1)C(=O)N(Cc1cncc(N(C)C3CCC3)n1)[C@@H]1CN(C3CC4(CC4)C3)C[C@H]1O2] | | | | | | +++ |
| 1323 | 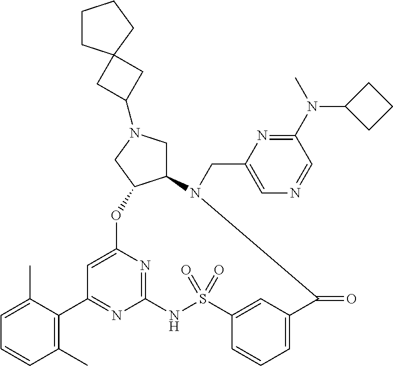 [SMILES: Cc1cccc(C)c1-c1cc2nc(n1)NS(=O)(=O)c1cccc(c1)C(=O)N(Cc1cncc(N(C)C3CCC3)n1)[C@@H]1CN(C3CC4(CCCC4)C3)C[C@H]1O2] | | | | | | ++ |
| 1743 | 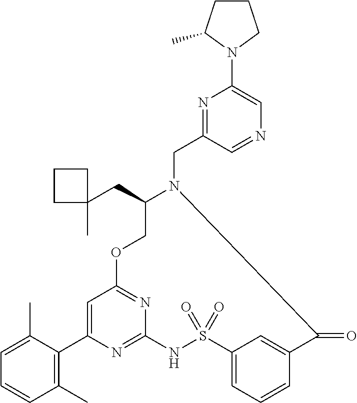 [SMILES: Cc1cccc(C)c1-c1cc2nc(n1)NS(=O)(=O)c1cccc(c1)C(=O)N(Cc1cncc(N3CCC[C@H]3C)n1)[C@H](CC1(C)CCC1)CO2] | | | | | | +++ |
| 1744 | 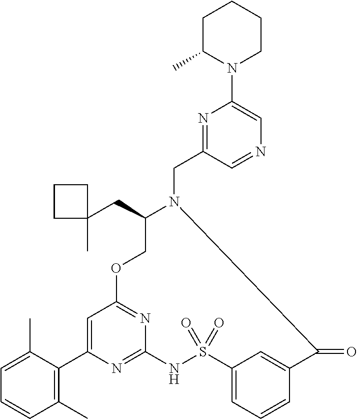 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1(C)CCC1)CO2] | | | | | | +++ |
| 1745 | 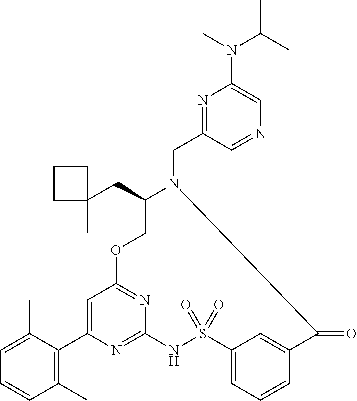 [SMILES: Cc1cccc(C)c1-c1cc2nc(n1)NS(=O)(=O)c1cccc(c1)C(=O)N(Cc1cncc(N(C)C(C)C)n1)[C@H](CC1(C)CCC1)CO2] | | | | | | +++ |
| 1746 | 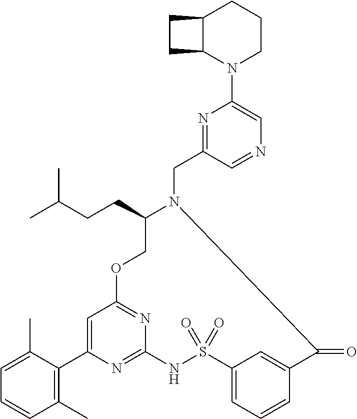 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CCC(C)C)CO2] | | | | | | +++ |
| 1747 | 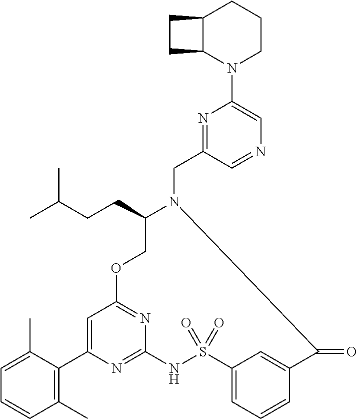 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CCC(C)C)CO2] | | | | | | +++ |
| 1748 | 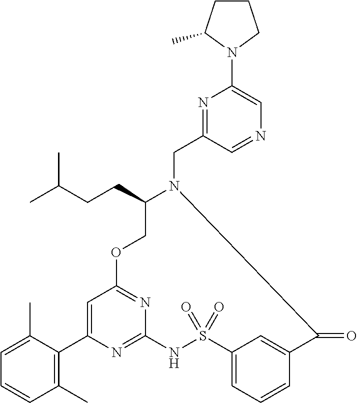 [SMILES: Cc1cccc(C)c1-c1cc2nc(n1)NS(=O)(=O)c1cccc(c1)C(=O)N(Cc1cncc(N3CCC[C@H]3C)n1)[C@H](CCC(C)C)CO2] | | | | | | +++ |
| 1749 | 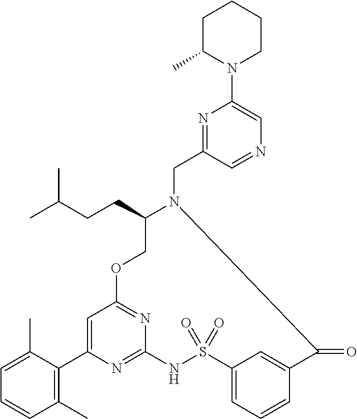 [SMILES: Cc1cccc(C)c1-c1cc2nc(n1)NS(=O)(=O)c1cccc(c1)C(=O)N(Cc1cncc(N3CCCC[C@H]3C)n1)[C@H](CCC(C)C)CO2] | | | | | | +++ |
| 1750 | 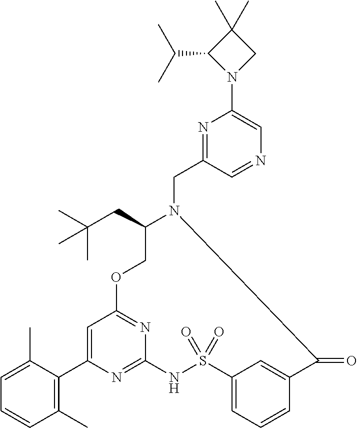 [SMILES: Cc1cccc(C)c1-c1cc2nc(n1)NS(=O)(=O)c1cccc(c1)C(=O)N(Cc1cncc(N3CC(C)(C)[C@H]3C(C)C)n1)[C@H](CC(C)(C)C)CO2] | | | | | | +++ |
| 1751 | 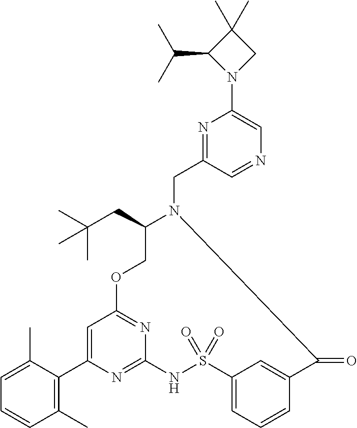 [SMILES: Cc1cccc(C)c1-c1cc2nc(n1)NS(=O)(=O)c1cccc(c1)C(=O)N(Cc1cncc(N3CC(C)(C)[C@@H]3C(C)C)n1)[C@H](CC(C)(C)C)CO2] | | | | | | +++ |
| 1752 | 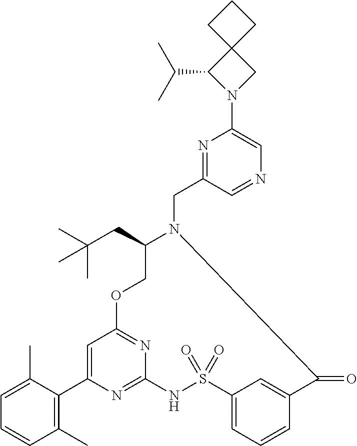 [SMILES: Cc1cccc(C)c1-c1cc2nc(n1)NS(=O)(=O)c1cccc(c1)C(=O)N(Cc1cncc(N3CC4(CCC4)[C@H]3C(C)C)n1)[C@H](CC(C)(C)C)CO2] | | | | | | +++ |
| 1753 | 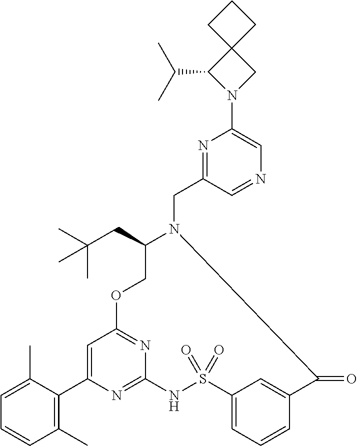 [SMILES: Cc1cccc(C)c1-c1cc2nc(n1)NS(=O)(=O)c1cccc(c1)C(=O)N(Cc1cncc(N3CC4(CCC4)[C@H]3C(C)C)n1)[C@H](CC(C)(C)C)CO2] | | | | | | +++ |
| 1754 | 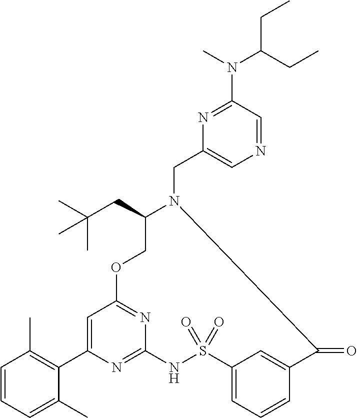 [SMILES: CCC(CC)N(C)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1755 | 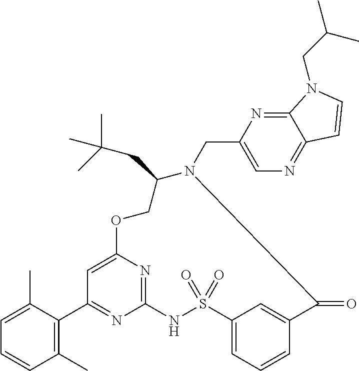 [SMILES: Cc1cccc(C)c1-c1cc2nc(n1)NS(=O)(=O)c1cccc(c1)C(=O)N(Cc1cnc3ccn(CC(C)C)c3n1)[C@H](CC(C)(C)C)CO2] | | | | | | +++ |
| 1756 | 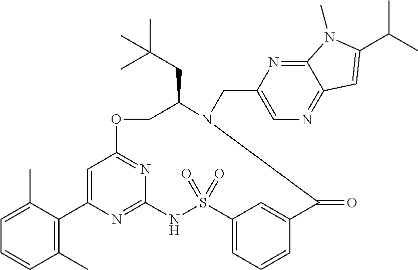 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C)CO2] | | | | | | +++ |
| 1757 | 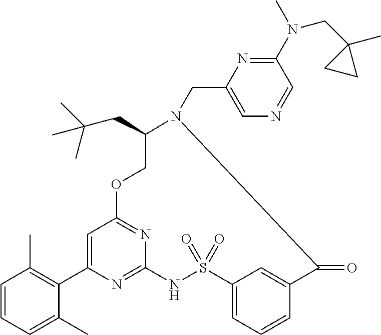 [SMILES: Cc1cccc(C)c1-c1cc2nc(n1)NS(=O)(=O)c1cccc(c1)C(=O)N(Cc1cncc(N(C)CC3(C)CC3)n1)[C@H](CC(C)(C)C)CO2] | | | | | | +++ |
| 1758 | 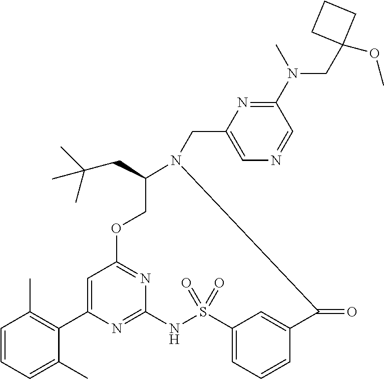 [SMILES: COC1(CN(C)c2cncc(CN3C(=O)c4cccc(c4)S(=O)(=O)Nc4nc(cc(-c5c(C)cccc5C)n4)OC[C@H]3CC(C)(C)C)n2)CCC1] | | | | | | +++ |
| 1759 | 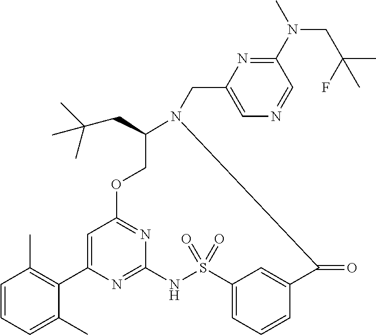 [SMILES: Cc1cccc(C)c1-c1cc2nc(n1)NS(=O)(=O)c1cccc(c1)C(=O)N(Cc1cncc(N(C)CC(C)(C)F)n1)[C@H](CC(C)(C)C)CO2] | | | | | | +++ |
| 1760 | 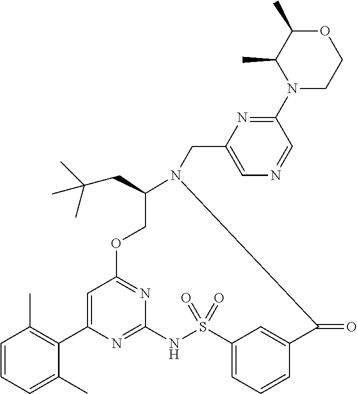 [SMILES: Cc1cccc(C)c1-c1cc2nc(n1)NS(=O)(=O)c1cccc(c1)C(=O)N(Cc1cncc(N3CCO[C@H](C)[C@@H]3C)n1)[C@H](CC(C)(C)C)CO2] | | | | | | +++ |
| 1761 | 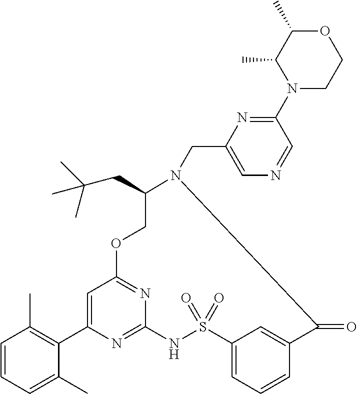 [SMILES: Cc1cccc(C)c1-c1cc2nc(n1)NS(=O)(=O)c1cccc(c1)C(=O)N(Cc1cncc(N3CCO[C@@H](C)[C@H]3C)n1)[C@H](CC(C)(C)C)CO2] | | | | | | +++ |
| 1762 | 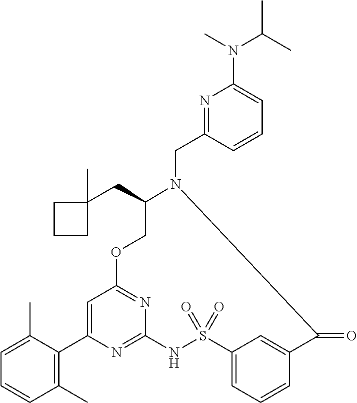 [SMILES: Cc1cccc(C)c1-c1cc2nc(n1)NS(=O)(=O)c1cccc(c1)C(=O)N(Cc1cccc(N(C)C(C)C)n1)[C@H](CC1(C)CCC1)CO2] | | | | | | +++ |
| 1763 | 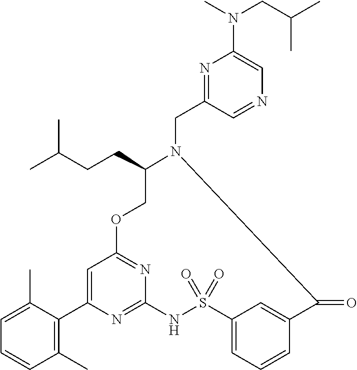 [SMILES: Cc1cccc(C)c1-c1cc2nc(n1)NS(=O)(=O)c1cccc(c1)C(=O)N(Cc1cncc(N(C)CC(C)C)n1)[C@H](CCC(C)C)CO2] | | | | | | +++ |
| 1764 | 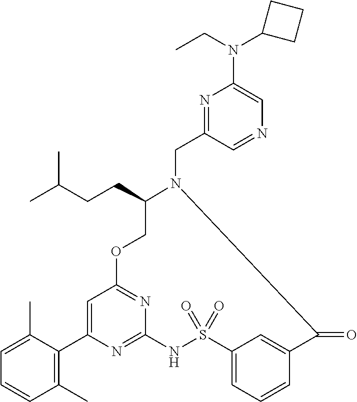 [SMILES: CCN(c1cncc(CN2C(=O)c3cccc(c3)S(=O)(=O)Nc3nc(cc(-c4c(C)cccc4C)n3)OC[C@H]2CCC(C)C)n1)C1CCC1] | | | | | | +++ |
| 1765 | 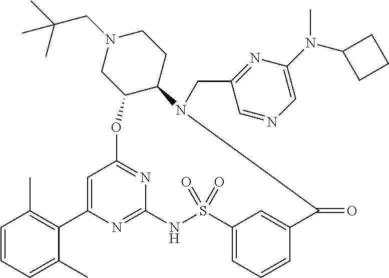 [SMILES: Cc1cccc(C)c1-c1cc2nc(n1)NS(=O)(=O)c1cccc(c1)C(=O)N(Cc1cncc(N(C)C3CCC3)n1)[C@@H]1CCN(CC(C)(C)C)C[C@H]1O2] | | | | | | ++ |
| 1766 | 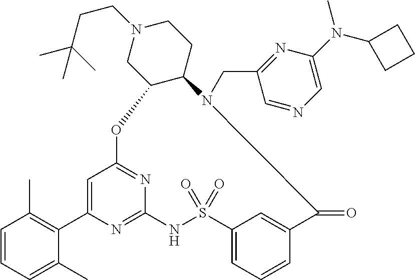 [SMILES: Cc1cccc(C)c1-c1cc2nc(n1)NS(=O)(=O)c1cccc(c1)C(=O)N(Cc1cncc(N(C)C3CCC3)n1)[C@@H]1CCN(CCC(C)(C)C)C[C@H]1O2] | | | | | | +++ |
| 1767 | 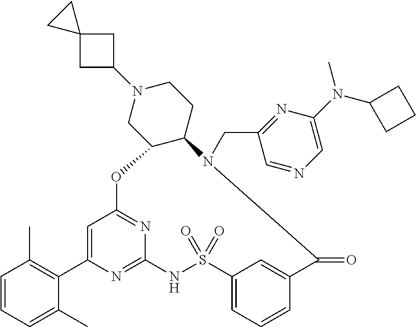 [SMILES: Cc1cccc(C)c1-c1cc2nc(n1)NS(=O)(=O)c1cccc(c1)C(=O)N(Cc1cncc(N(C)C3CCC3)n1)[C@@H]1CCN(C3CC4(CC4)C3)C[C@H]1O2] | | | | | | +++ |
| 1768 | 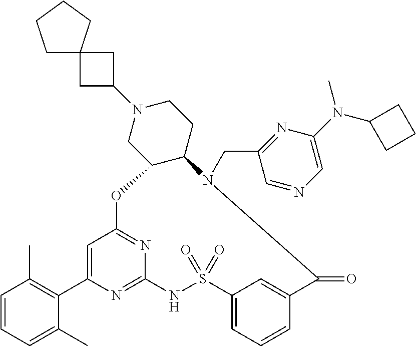 [SMILES: Cc1cccc(C)c1-c1cc2nc(n1)NS(=O)(=O)c1cccc(c1)C(=O)N(Cc1cncc(N(C)C3CCC3)n1)[C@@H]1CCN(C3CC4(CCCC4)C3)C[C@H]1O2] | | | | | | +++ |
| 1769 | 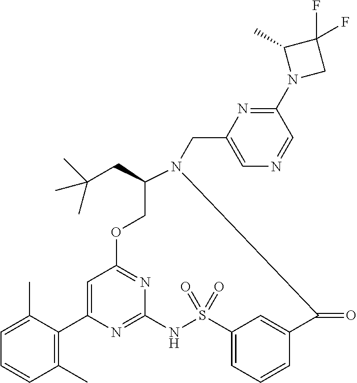 [SMILES: Cc1cccc(C)c1-c1cc2nc(n1)NS(=O)(=O)c1cccc(c1)C(=O)N(Cc1cncc(N3CC(F)(F)[C@H]3C)n1)[C@H](CC(C)(C)C)CO2] | | | | | | +++ |
| 1770 | 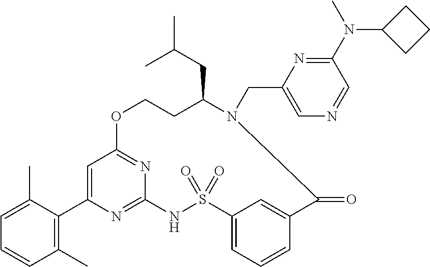 [SMILES: Cc1cccc(C)c1-c1cc2nc(n1)NS(=O)(=O)c1cccc(c1)C(=O)N(Cc1cncc(N(C)C3CCC3)n1)[C@H](CC(C)C)CCO2] | | | | | | +++ |
| 1771 | 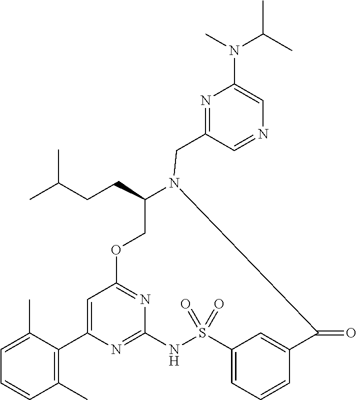 [SMILES: Cc1cccc(C)c1-c1cc2nc(n1)NS(=O)(=O)c1cccc(c1)C(=O)N(Cc1cncc(N(C)C(C)C)n1)[C@H](CCC(C)C)CO2] | | | | | | +++ |
| 1772 | 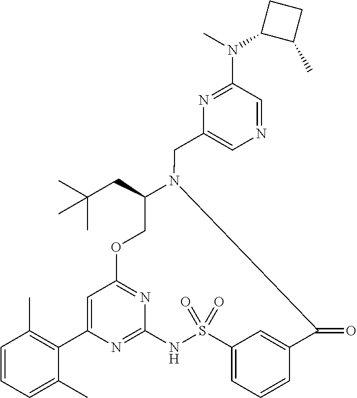 [SMILES: Cc1cccc(C)c1-c1cc2nc(n1)NS(=O)(=O)c1cccc(c1)C(=O)N(Cc1cncc(N(C)[C@@H]3CC[C@@H]3C)n1)[C@H](CC(C)(C)C)CO2] | | | | | | +++ |
| 1773 | 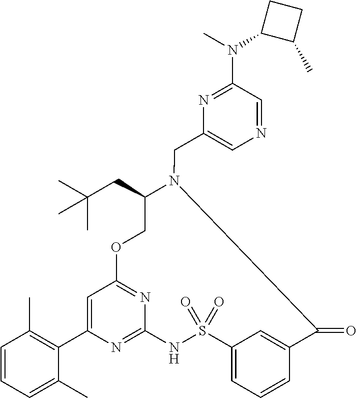 [SMILES: Cc1cccc(C)c1-c1cc2nc(n1)NS(=O)(=O)c1cccc(c1)C(=O)N(Cc1cncc(N(C)[C@@H]3CC[C@@H]3C)n1)[C@H](CC(C)(C)C)CO2] | | | | | | +++ |
| 1774 | 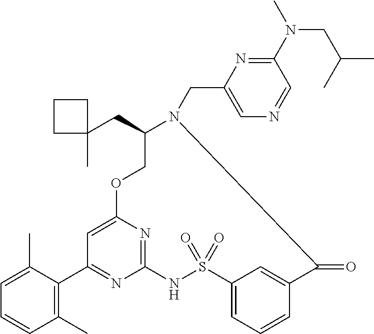 [SMILES: Cc1cccc(C)c1-c1cc2nc(n1)NS(=O)(=O)c1cccc(c1)C(=O)N(Cc1cncc(N(C)CC(C)C)n1)[C@H](CC1(C)CCC1)CO2] | | | | | | +++ |
| 1775 | 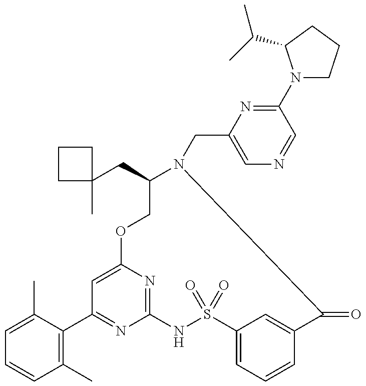 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](CC1(C)CCC1)CO2] | | | | | | +++ |
| 1776 | 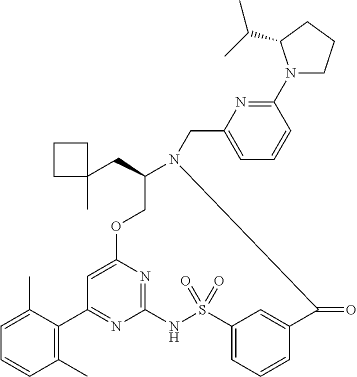 [SMILES: Cc1cccc(C)c1-c1cc2nc(n1)NS(=O)(=O)c1cccc(c1)C(=O)N(Cc1cccc(N3CCC[C@H]3C(C)C)n1)[C@H](CC1(C)CCC1)CO2] | | | | | | +++ |
| 1777 | 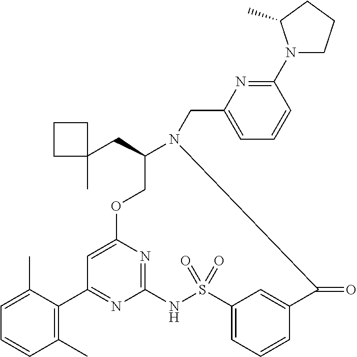 [SMILES: Cc1cccc(C)c1-c1cc2nc(n1)NS(=O)(=O)c1cccc(c1)C(=O)N(Cc1cccc(N3CCC[C@H]3C)n1)[C@H](CC1(C)CCC1)CO2] | | | | | | +++ |
| 1778 | 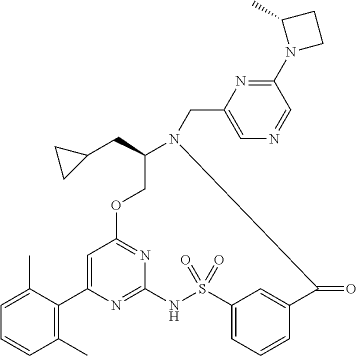 [SMILES: Cc1cccc(C)c1-c1cc2nc(n1)NS(=O)(=O)c1cccc(c1)C(=O)N(Cc1cncc(N3CC[C@H]3C)n1)[C@H](CC1CC1)CO2] | | | | | | +++ |
| 1779 | 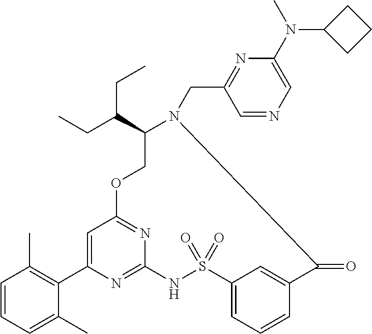 [SMILES: CCC(CC)[C@@H]1COc2cc(-c3c(C)cccc3C)nc(n2)NS(=O)(=O)c2cccc(c2)C(=O)N1Cc1cncc(N(C)C2CCC2)n1] | | | | | | +++ |
| 1780 | 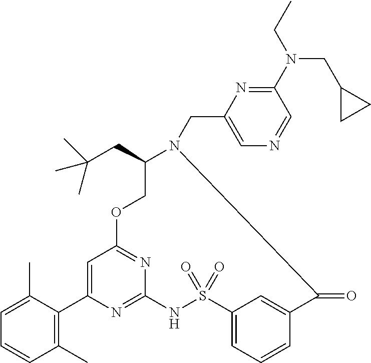 [SMILES: CCN(CC1CC1)c1cncc(CN2C(=O)c3cccc(c3)S(=O)(=O)Nc3nc(cc(-c4c(C)cccc4C)n3)OC[C@H]2CC(C)(C)C)n1] | | | | | | +++ |
| 1781 | 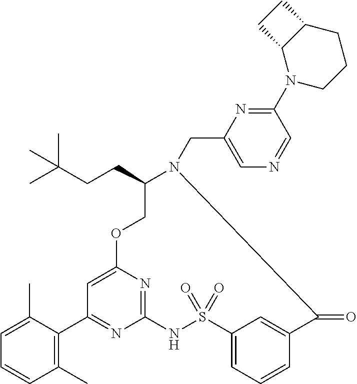 [SMILES: Cc1cccc(C)c1-c1cc2nc(n1)NS(=O)(=O)c1cccc(c1)C(=O)N(Cc1cncc(N3CCC[C@H]4CC[C@H]43)n1)[C@H](CCC(C)(C)C)CO2] | | | | | | +++ |
| 1782 | 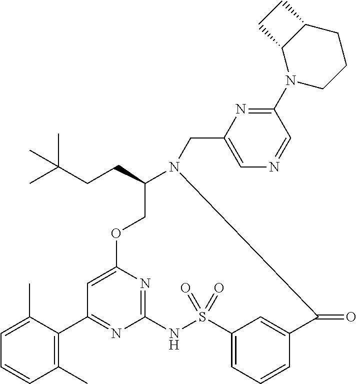 [SMILES: Cc1cccc(C)c1-c1cc2nc(n1)NS(=O)(=O)c1cccc(c1)C(=O)N(Cc1cncc(N3CCC[C@H]4CC[C@H]43)n1)[C@H](CCC(C)(C)C)CO2] | | | | | | +++ |
| 1783 | 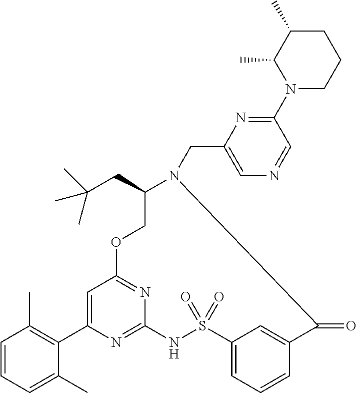 [SMILES: Cc1cccc(C)c1-c1cc2nc(n1)NS(=O)(=O)c1cccc(c1)C(=O)N(Cc1cncc(N3CCC[C@@H](C)[C@H]3C)n1)[C@H](CC(C)(C)C)CO2] | | | | | | +++ |
| 1784 | 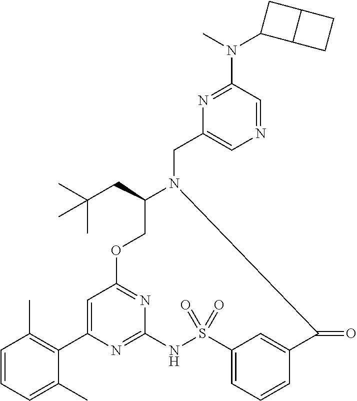 [SMILES: Cc1cccc(C)c1-c1cc2nc(n1)NS(=O)(=O)c1cccc(c1)C(=O)N(Cc1cncc(N(C)C3CC4CCC43)n1)[C@H](CC(C)(C)C)CO2] | | | | | | +++ |
| 1785 | 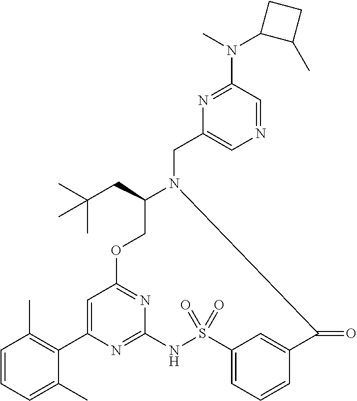 [SMILES: Cc1cccc(C)c1-c1cc2nc(n1)NS(=O)(=O)c1cccc(c1)C(=O)N(Cc1cncc(N(C)C3CCC3C)n1)[C@H](CC(C)(C)C)CO2] | | | | | | +++ |
| 1786 | 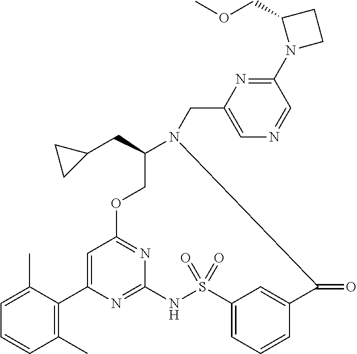 [SMILES: COC[C@@H]1CCN1c1cncc(CN2C(=O)c3cccc(c3)S(=O)(=O)Nc3nc(cc(-c4c(C)cccc4C)n3)OC[C@H]2CC2CC2)n1] | | | | | | +++ |
| 1787 | 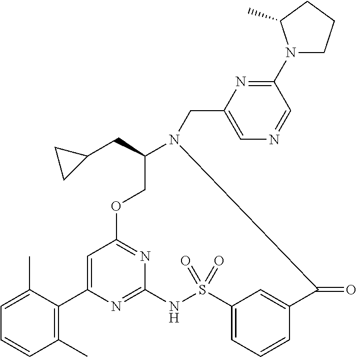 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CC1)CO2] | | | | | | +++ |
| 1788 | 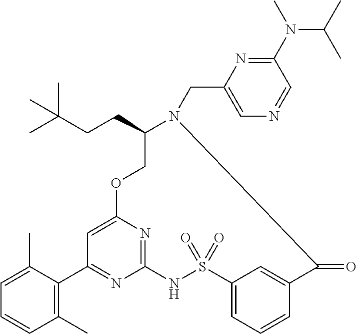 [SMILES: Cc1cccc(C)c1-c1cc2nc(n1)NS(=O)(=O)c1cccc(c1)C(=O)N(Cc1cncc(N(C)C(C)C)n1)[C@H](CCC(C)(C)C)CO2] | | | | | | +++ |
| 1789 | 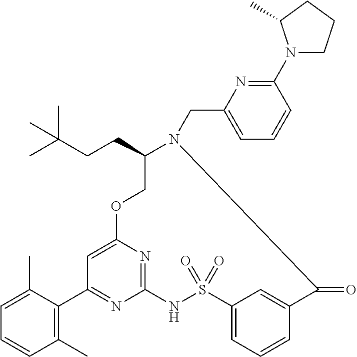 [SMILES: Cc1cccc(C)c1-c1cc2nc(n1)NS(=O)(=O)c1cccc(c1)C(=O)N(Cc1cccc(N3CCC[C@H]3C)n1)[C@H](CCC(C)(C)C)CO2] | | | | | | +++ |
| 1790 | 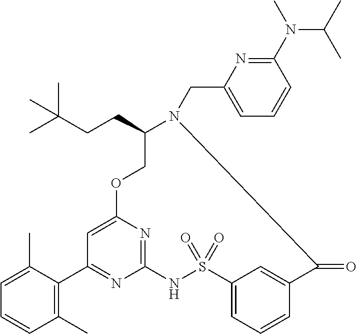 [SMILES: Cc1cccc(C)c1-c1cc2nc(n1)NS(=O)(=O)c1cccc(c1)C(=O)N(Cc1cccc(N(C)C(C)C)n1)[C@H](CCC(C)(C)C)CO2] | | | | | | +++ |
| 1791 | 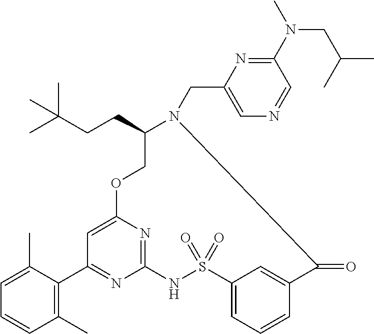 [SMILES: Cc1cccc(C)c1-c1cc2nc(n1)NS(=O)(=O)c1cccc(c1)C(=O)N(Cc1cncc(N(C)CC(C)C)n1)[C@H](CCC(C)(C)C)CO2] | | | | | | +++ |
| 1792 | 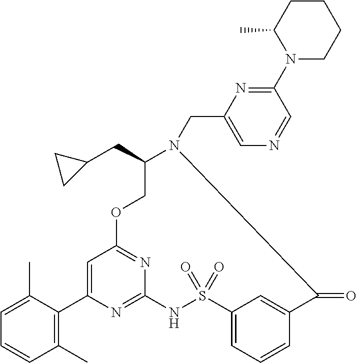 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CC1)CO2] | | | | | | +++ |
| 1793 | 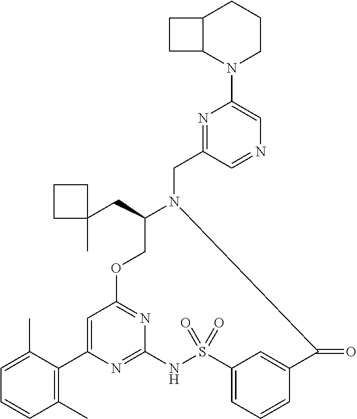 [SMILES: Cc1cccc(C)c1-c1cc2nc(n1)NS(=O)(=O)c1cccc(c1)C(=O)N(Cc1cncc(N3CCCC4CCC43)n1)[C@H](CC1(C)CCC1)CO2] | | | | | | +++ |
| 1794 | 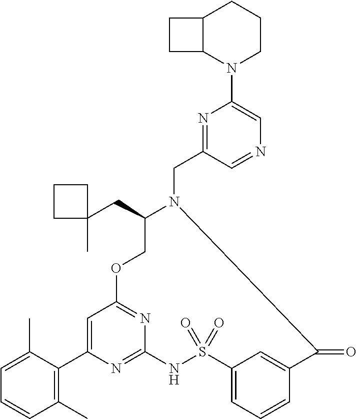 [SMILES: Cc1cccc(C)c1-c1cc2nc(n1)NS(=O)(=O)c1cccc(c1)C(=O)N(Cc1cncc(N3CCCC4CCC43)n1)[C@H](CC1(C)CCC1)CO2] | | | | | | +++ |
| 1795 | 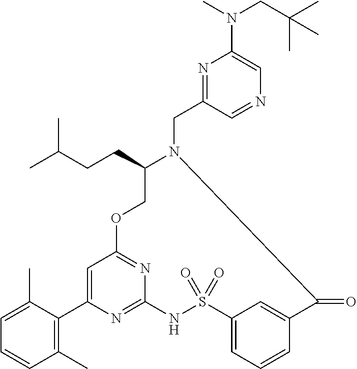 [SMILES: Cc1cccc(C)c1-c1cc2nc(n1)NS(=O)(=O)c1cccc(c1)C(=O)N(Cc1cncc(N(C)CC(C)(C)C)n1)[C@H](CCC(C)C)CO2] | | | | | | +++ |
| 1796 | 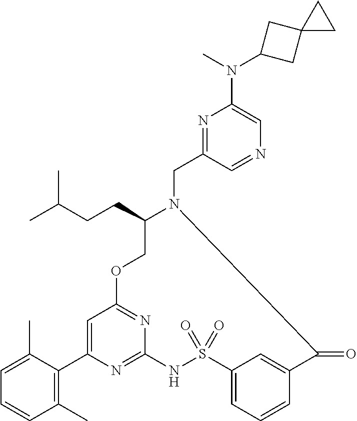 [SMILES: Cc1cccc(C)c1-c1cc2nc(n1)NS(=O)(=O)c1cccc(c1)C(=O)N(Cc1cncc(N(C)C3CC4(CC4)C3)n1)[C@H](CCC(C)C)CO2] | | | | | | +++ |
| 1797 | 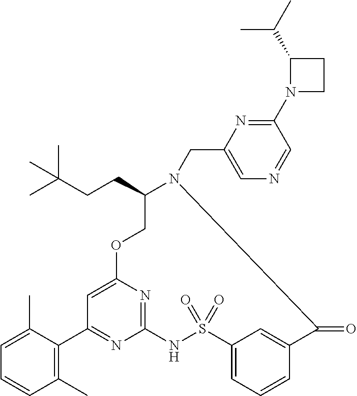 [SMILES: Cc1cccc(C)c1-c1cc2nc(n1)NS(=O)(=O)c1cccc(c1)C(=O)N(Cc1cncc(N3CC[C@H]3C(C)C)n1)[C@H](CCC(C)(C)C)CO2] | | | | | | +++ |
| 1798 | 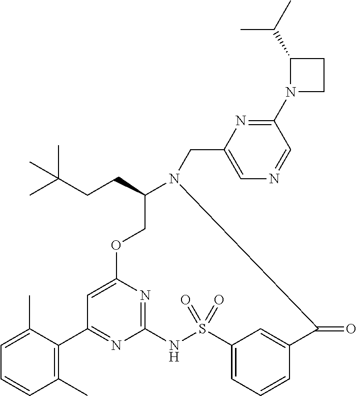 [SMILES: Cc1cccc(C)c1-c1cc2nc(n1)NS(=O)(=O)c1cccc(c1)C(=O)N(Cc1cncc(N3CC[C@H]3C(C)C)n1)[C@H](CCC(C)(C)C)CO2] | | | | | | +++ |
| 1799 | 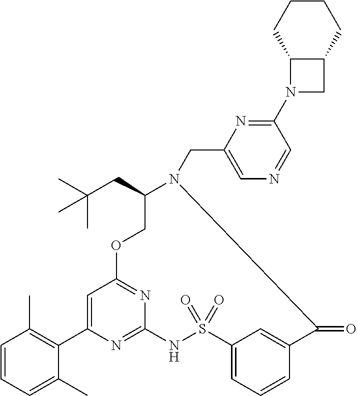 [SMILES: Cc1cccc(C)c1-c1cc2nc(n1)NS(=O)(=O)c1cccc(c1)C(=O)N(Cc1cncc(N3C[C@H]4CCCC[C@H]43)n1)[C@H](CC(C)(C)C)CO2] | | | | | | +++ |
| 1800 | 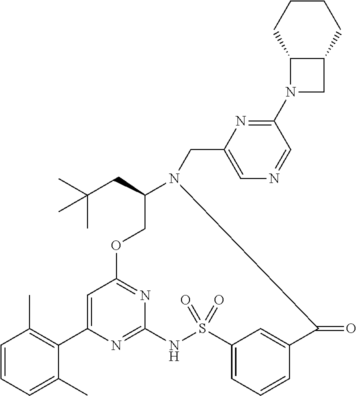 [SMILES: Cc1cccc(C)c1-c1cc2nc(n1)NS(=O)(=O)c1cccc(c1)C(=O)N(Cc1cncc(N3C[C@H]4CCCC[C@H]43)n1)[C@H](CC(C)(C)C)CO2] | | | | | | +++ |
| 1801 | 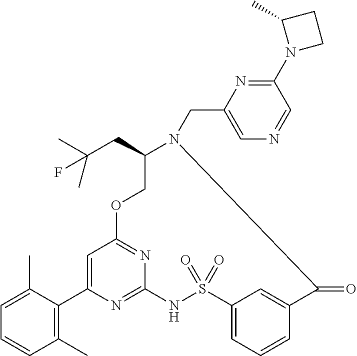 [SMILES: Cc1cccc(C)c1-c1cc2nc(n1)NS(=O)(=O)c1cccc(c1)C(=O)N(Cc1cncc(N3CC[C@H]3C)n1)[C@H](CC(C)(C)F)CO2] | | | | | | +++ |
| 1802 | 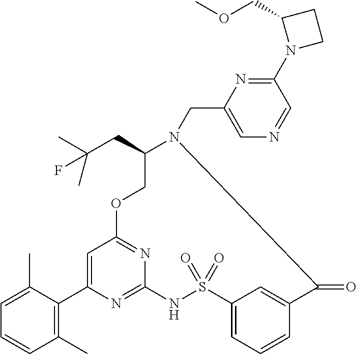 [SMILES: COC[C@@H]1CCN1c1cncc(CN2C(=O)c3cccc(c3)S(=O)(=O)Nc3nc(cc(-c4c(C)cccc4C)n3)OC[C@H]2CC(C)(C)F)n1] | | | | | | +++ |
| 1803 | 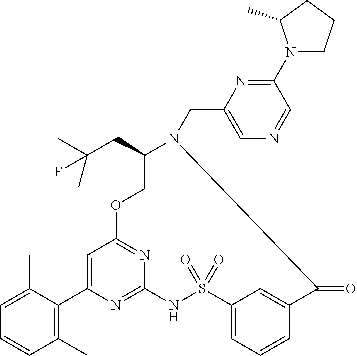 [SMILES: Cc1cccc(C)c1-c1cc2nc(n1)NS(=O)(=O)c1cccc(c1)C(=O)N(Cc1cncc(N3CCC[C@H]3C)n1)[C@H](CC(C)(C)F)CO2] | | | | | | +++ |
| 1804 | 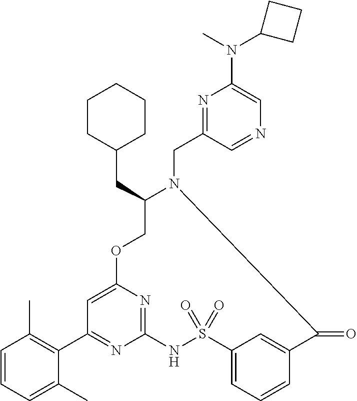 [SMILES: Cc1cccc(C)c1-c1cc2nc(n1)NS(=O)(=O)c1cccc(c1)C(=O)N(Cc1cncc(N(C)C3CCC3)n1)[C@H](CC1CCCCC1)CO2] | | | | | | +++ |
| 1805 | 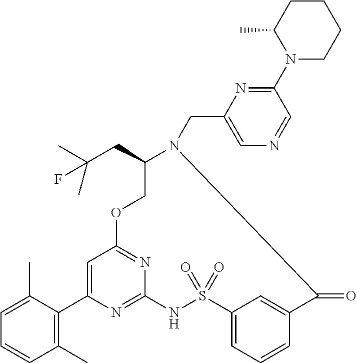 [SMILES: Cc1cccc(C)c1-c1cc2nc(n1)NS(=O)(=O)c1cccc(c1)C(=O)N(Cc1cncc(N3CCCC[C@H]3C)n1)[C@H](CC(C)(C)F)CO2] | | | | | | +++ |
| 1324 | 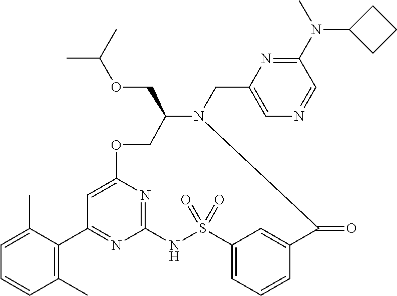 [SMILES: Cc1cccc(C)c1-c1cc2nc(n1)NS(=O)(=O)c1cccc(c1)C(=O)N(Cc1cncc(N(C)C3CCC3)n1)[C@H](COC(C)C)CO2] | | | | | | +++ |
| 1806 | 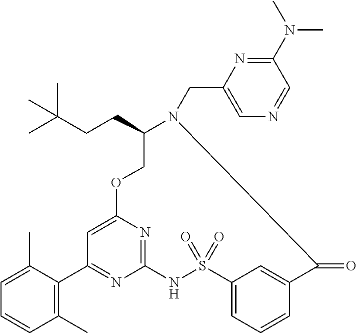 [SMILES: Cc1cccc(C)c1-c1cc2nc(n1)NS(=O)(=O)c1cccc(c1)C(=O)N(Cc1cncc(N(C)C)n1)[C@H](CCC(C)(C)C)CO2] | | | | | | +++ |
| 1807 | 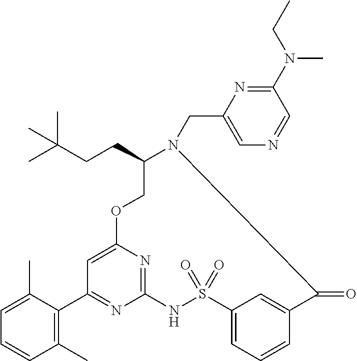 [SMILES: CCN(C)c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1808 | 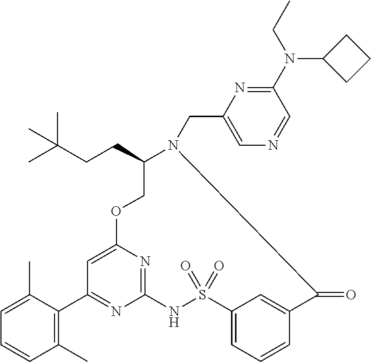 [SMILES: CCN(c1cncc(CN2C(=O)c3cccc(c3)S(=O)(=O)Nc3nc(cc(-c4c(C)cccc4C)n3)OC[C@H]2CCC(C)(C)C)n1)C1CCC1] | | | | | | +++ |
| 1809 | 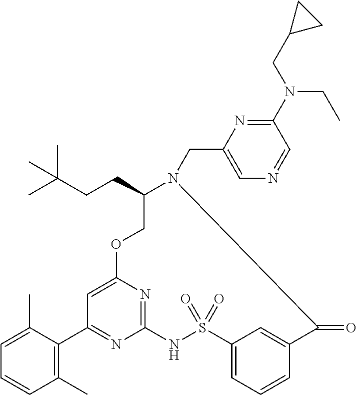 [SMILES: CCN(CC1CC1)c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1810 | 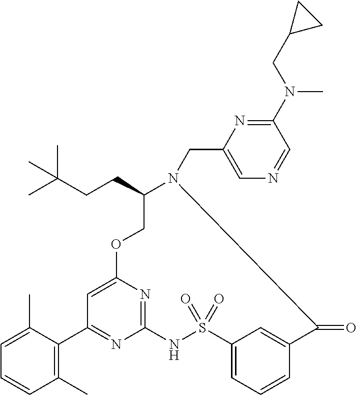 [SMILES: Cc1cccc(C)c1-c1cc2nc(n1)NS(=O)(=O)c1cccc(c1)C(=O)N(Cc1cncc(N(C)CC3CC3)n1)[C@H](CCC(C)(C)C)CO2] | | | | | | +++ |
| 1811 | 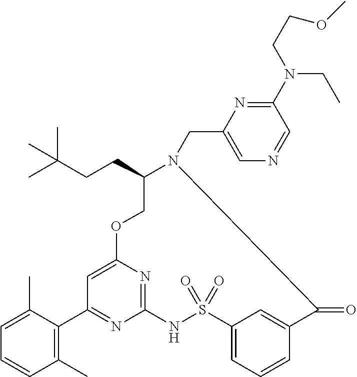 [SMILES: CCN(CCOC)c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1812 | 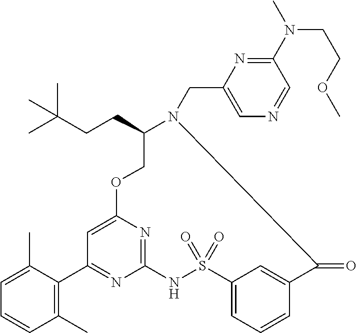 [SMILES: COCCN(C)c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1813 | 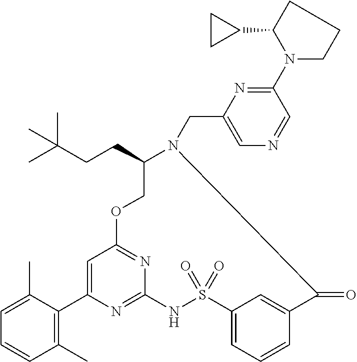 [SMILES: Cc1cccc(C)c1-c1cc2nc(n1)NS(=O)(=O)c1cccc(c1)C(=O)N(Cc1cncc(N3CCC[C@H]3C3CC3)n1)[C@H](CCC(C)(C)C)CO2] | | | | | | +++ |
| 1814 | 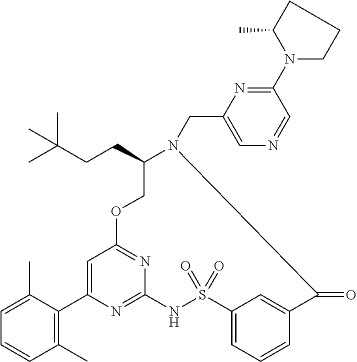 [SMILES: Cc1cccc(C)c1-c1cc2nc(n1)NS(=O)(=O)c1cccc(c1)C(=O)N(Cc1cncc(N3CCC[C@H]3C)n1)[C@H](CCC(C)(C)C)CO2] | | | | | | +++ |
| 1815 | 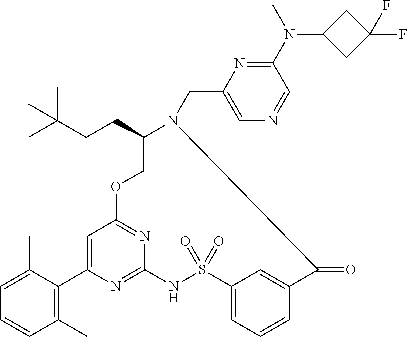 [SMILES: Cc1cccc(C)c1-c1cc2nc(n1)NS(=O)(=O)c1cccc(c1)C(=O)N(Cc1cncc(N(C)C3CC(F)(F)C3)n1)[C@H](CCC(C)(C)C)CO2] | | | | | | +++ |
| 1816 | 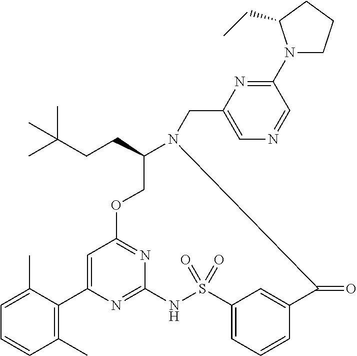 [SMILES: CC[C@@H]1CCCN1c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1817 | 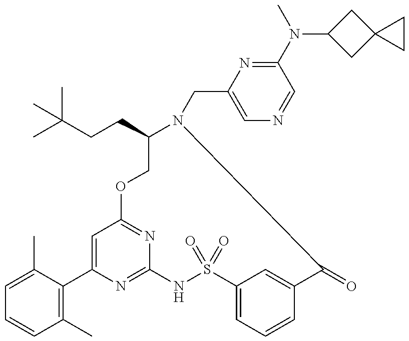 [SMILES: Cc1cccc(C)c1-c1cc2nc(n1)NS(=O)(=O)c1cccc(c1)C(=O)N(Cc1cncc(N(C)C3CC4(CC4)C3)n1)[C@H](CCC(C)(C)C)CO2] | | | | | | +++ |
| 1818 | 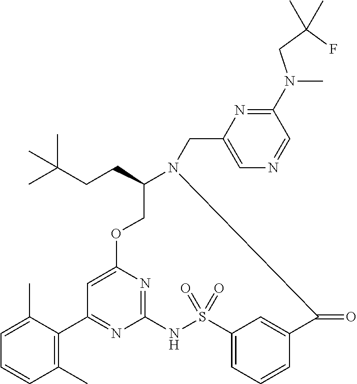 [SMILES: Cc1cccc(C)c1-c1cc2nc(n1)NS(=O)(=O)c1cccc(c1)C(=O)N(Cc1cncc(N(C)CC(C)(C)F)n1)[C@H](CCC(C)(C)C)CO2] | | | | | | +++ |
| 1819 | 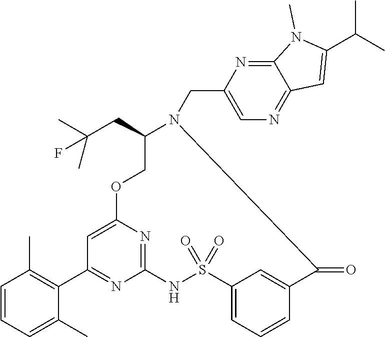 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)F)CO2] | | | | | | +++ |
| 1820 | 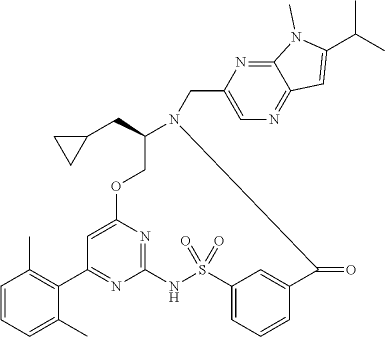 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CC1)CO2] | | | | | | +++ |
| 1821 | 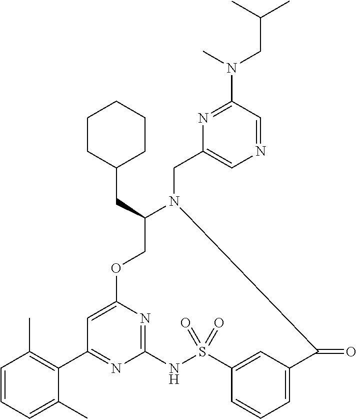 [SMILES: Cc1cccc(C)c1-c1cc2nc(n1)NS(=O)(=O)c1cccc(c1)C(=O)N(Cc1cncc(N(C)CC(C)C)n1)[C@H](CC1CCCCC1)CO2] | | | | | | +++ |
| 1822 | 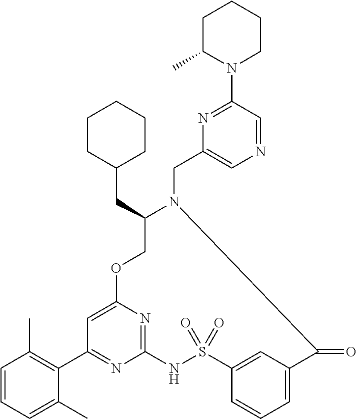 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CCCCC1)CO2] | | | | | | +++ |
| 1823 | 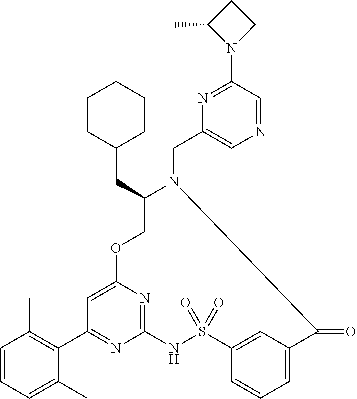 [SMILES: Cc1cccc(C)c1-c1cc2nc(n1)NS(=O)(=O)c1cccc(c1)C(=O)N(Cc1cncc(N3CC[C@H]3C)n1)[C@H](CC1CCCCC1)CO2] | | | | | | +++ |
| 1824 | 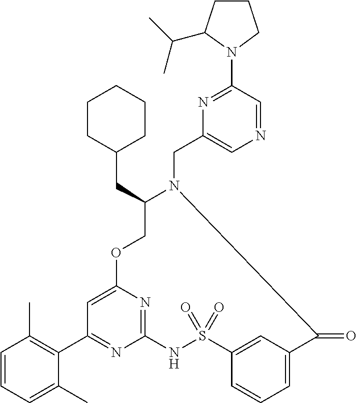 [SMILES: Cc1cccc(C)c1-c1cc2nc(n1)NS(=O)(=O)c1cccc(c1)C(=O)N(Cc1cncc(N3CCCC3C(C)C)n1)[C@H](CC1CCCCC1)CO2] | | | | | | +++ |
| 1825 | 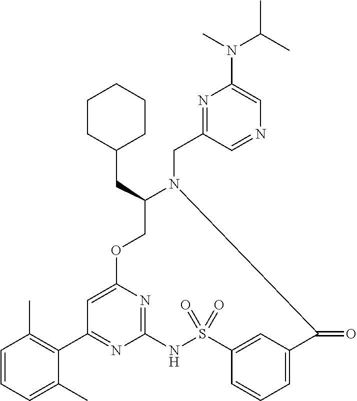 [SMILES: Cc1cccc(C)c1-c1cc2nc(n1)NS(=O)(=O)c1cccc(c1)C(=O)N(Cc1cncc(N(C)C(C)C)n1)[C@H](CC1CCCCC1)CO2] | | | | | | +++ |
| 1826 | 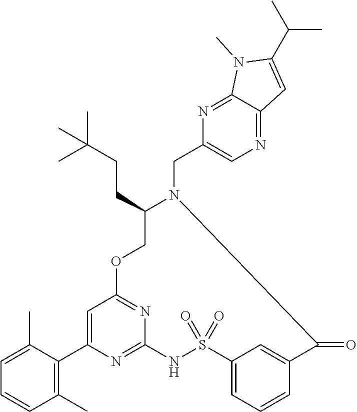 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC(C)(C)C)CO2] | | | | | | +++ |
| 1827 | 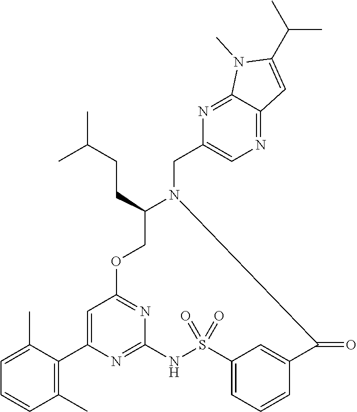 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC(C)C)CO2] | | | | | | +++ |
| 1828 | 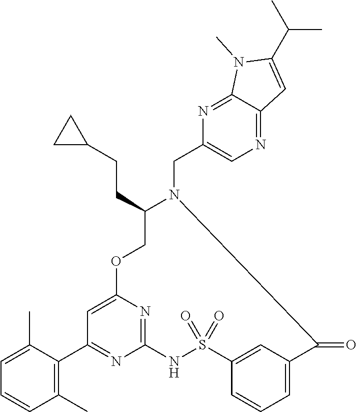 [SMILES: Cc1cccc(C)c1-c1cc2nc(n1)NS(=O)(=O)c1cccc(c1)C(=O)N(Cc1cnc3cc(C(C)C)n(C)c3n1)[C@H](CCC1CC1)CO2] | | | | | | +++ |
| 1829 | 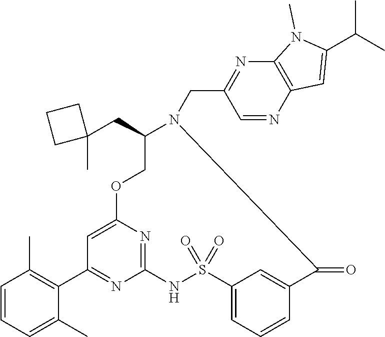 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(C)CCC1)CO2] | | | | | | +++ |
| 1830 | 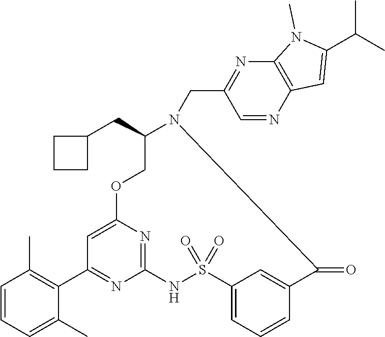 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCC1)CO2] | | | | | | +++ |
| 1831 | 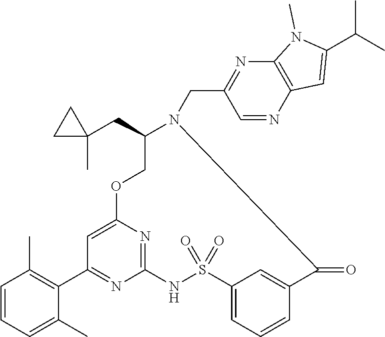 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(C)CC1)CO2] | | | | | | +++ |
| 1832 | 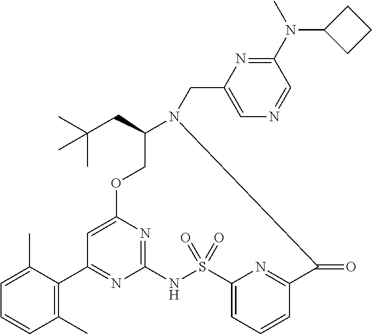 [SMILES: Cc1cccc(C)c1-c1cc2nc(n1)NS(=O)(=O)c1cccc(n1)C(=O)N(Cc1cncc(N(C)C3CCC3)n1)[C@H](CC(C)(C)C)CO2] | | | | | | +++ |
| 1833 | 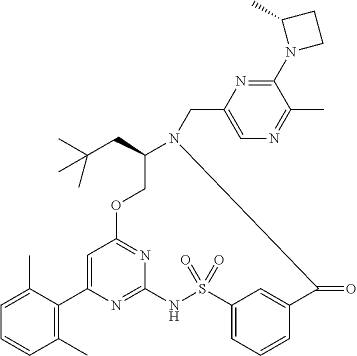 [SMILES: Cc1cccc(C)c1-c1cc2nc(n1)NS(=O)(=O)c1cccc(c1)C(=O)N(Cc1cnc(C)c(N3CC[C@H]3C)n1)[C@H](CC(C)(C)C)CO2] | | | | | | +++ |
| 1834 | 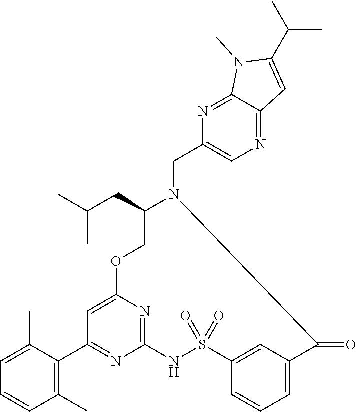 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)C)CO2] | | | | | | +++ |
| 1835 | 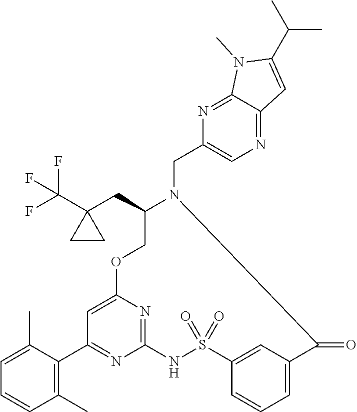 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1(C(F)(F)F)CC1)CO2] | | | | | | +++ |
| 1836 | 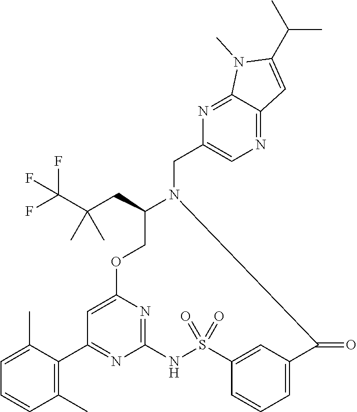 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC(C)(C)C(F)(F)F)CO2] | | | | | | +++ |
| 1325 | 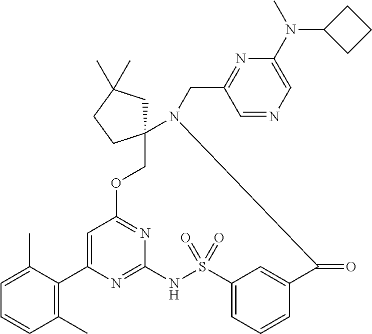 [SMILES: Cc1cccc(C)c1-c1cc2nc(n1)NS(=O)(=O)c1cccc(c1)C(=O)N(Cc1cncc(N(C)C3CCC3)n1)[C@]1(CCC(C)(C)C1)CO2] | | | | | | +++ |
| 1326 | 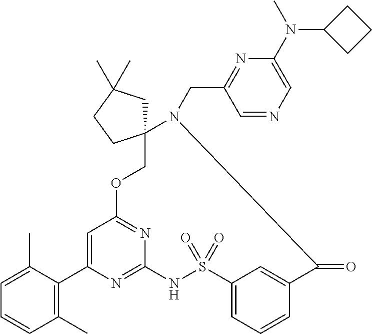 [SMILES: Cc1cccc(C)c1-c1cc2nc(n1)NS(=O)(=O)c1cccc(c1)C(=O)N(Cc1cncc(N(C)C3CCC3)n1)[C@]1(CCC(C)(C)C1)CO2] | | | | | | +++ |
| 1837 | 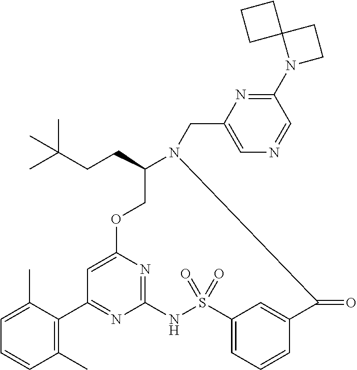 [SMILES: Cc1cccc(C)c1-c1cc2nc(n1)NS(=O)(=O)c1cccc(c1)C(=O)N(Cc1cncc(N3CCC34CCC4)n1)[C@H](CCC(C)(C)C)CO2] | | | | | | +++ |
| 1838 | 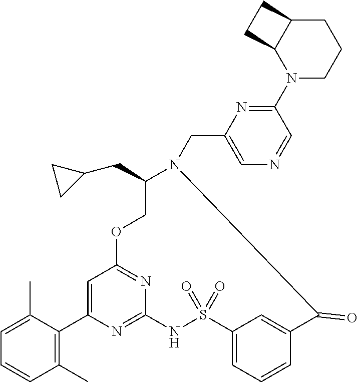 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CC1CC1)CO2] | | | | | | +++ |
| 1839 | 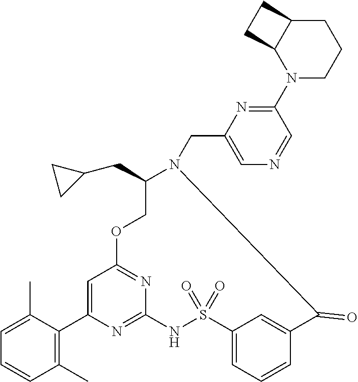 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CC1CC1)CO2] | | | | | | +++ |
| 1840 | 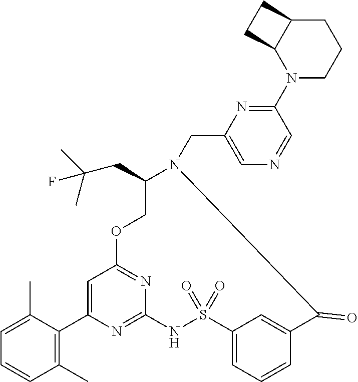 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CC(C)(C)F)CO2] | | | | | | +++ |
| 1841 | 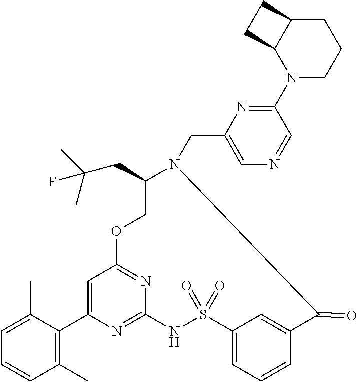 [SMILES: Cc1cccc(C)c1-c1cc2nc(n1)NS(=O)(=O)c1cccc(c1)C(=O)N(Cc1cncc(N3CCC[C@@H]4CC[C@@H]43)n1)[C@H](CC(C)(C)F)CO2] | | | | | | +++ |
| 1842 | 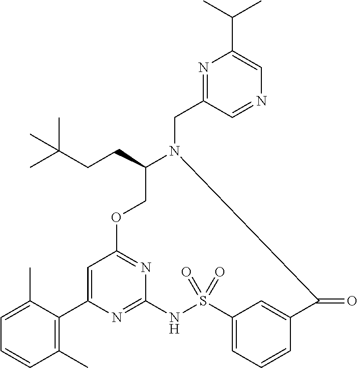 [SMILES: Cc1cccc(C)c1-c1cc2nc(n1)NS(=O)(=O)c1cccc(c1)C(=O)N(Cc1cncc(C(C)C)n1)[C@H](CCC(C)(C)C)CO2] | | | | | | +++ |
| 1843 | 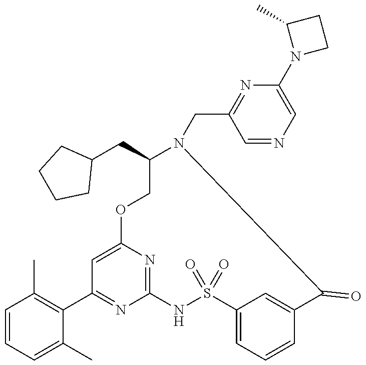 [SMILES: Cc1cccc(C)c1-c1cc2nc(n1)NS(=O)(=O)c1cccc(c1)C(=O)N(Cc1cncc(N3CC[C@H]3C)n1)[C@H](CC1CCCC1)CO2] | | | | | | +++ |
| 1844 | 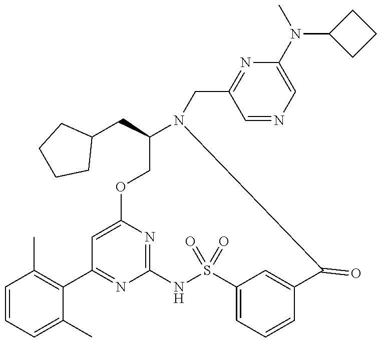 [SMILES: Cc1cccc(C)c1-c1cc2nc(n1)NS(=O)(=O)c1cccc(c1)C(=O)N(Cc1cncc(N(C)C3CCC3)n1)[C@H](CC1CCCC1)CO2] | | | | | | +++ |
| 1845 | 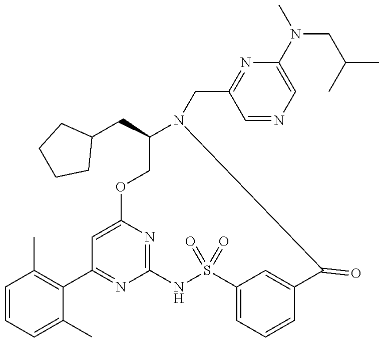 [SMILES: Cc1cccc(C)c1-c1cc2nc(n1)NS(=O)(=O)c1cccc(c1)C(=O)N(Cc1cncc(N(C)CC(C)C)n1)[C@H](CC1CCCC1)CO2] | | | | | | +++ |
| 1846 | 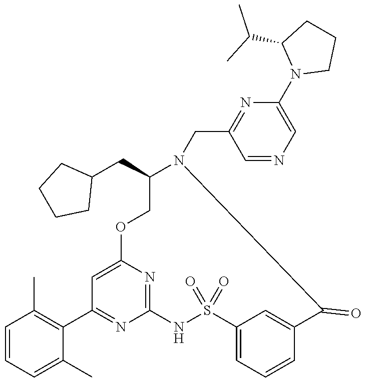 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](CC1CCCC1)CO2] | | | | | | +++ |
| 1847 | 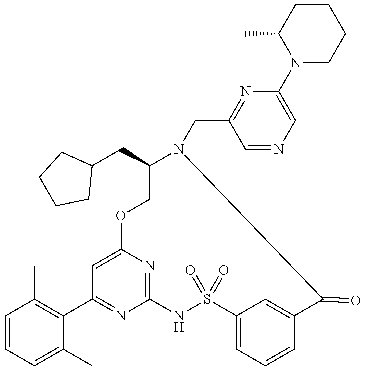 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CCCC1)CO2] | | | | | | +++ |
| 1848 | 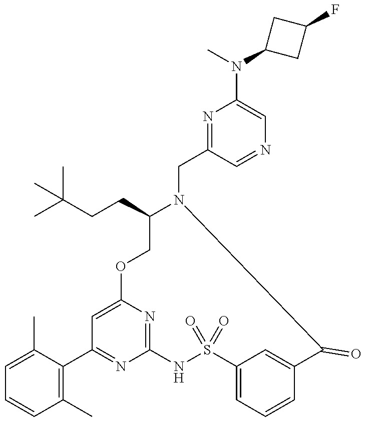 [SMILES: Cc1cccc(C)c1-c1cc2nc(n1)NS(=O)(=O)c1cccc(c1)C(=O)N(Cc1cncc(N(C)[C@H]3C[C@@H](F)C3)n1)[C@H](CCC(C)(C)C)CO2] | | | | | | +++ |
| 1849 | 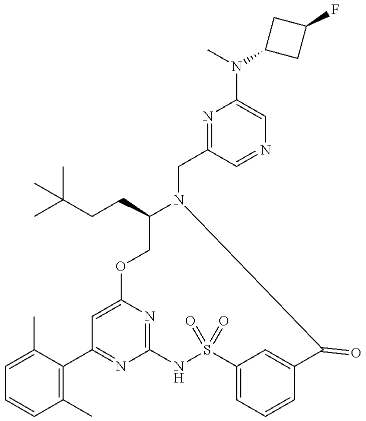 [SMILES: Cc1cccc(C)c1-c1cc2nc(n1)NS(=O)(=O)c1cccc(c1)C(=O)N(Cc1cncc(N(C)[C@H]3C[C@H](F)C3)n1)[C@H](CCC(C)(C)C)CO2] | | | | | | +++ |
| 1850 | 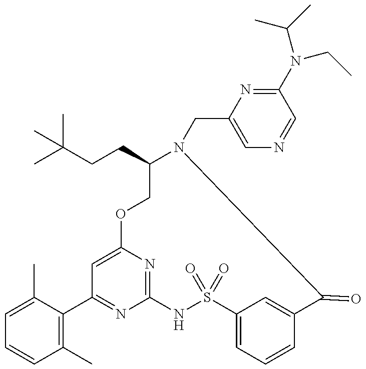 [SMILES: CCN(c1cncc(CN2C(=O)c3cccc(c3)S(=O)(=O)Nc3nc(cc(-c4c(C)cccc4C)n3)OC[C@H]2CCC(C)(C)C)n1)C(C)C] | | | | | | +++ |
| 1851 | 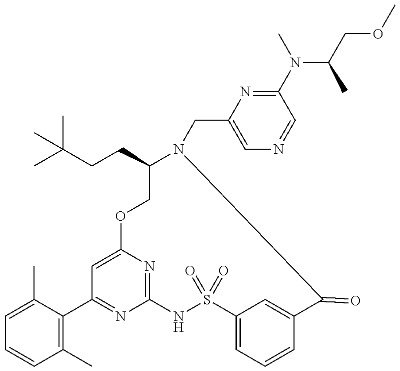 [SMILES: COC[C@@H](C)N(C)c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1852 | 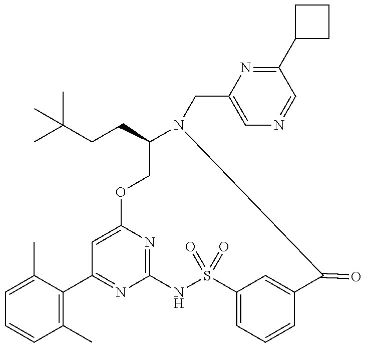 [SMILES: Cc1cccc(C)c1-c1cc2nc(n1)NS(=O)(=O)c1cccc(c1)C(=O)N(Cc1cncc(C3CCC3)n1)[C@H](CCC(C)(C)C)CO2] | | | | | | +++ |
| 1327 | 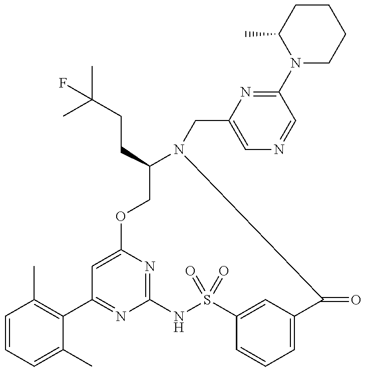 [SMILES: Cc1cccc(C)c1-c1cc2nc(n1)NS(=O)(=O)c1cccc(c1)C(=O)N(Cc1cncc(N3CCCC[C@H]3C)n1)[C@H](CCC(C)(C)F)CO2] | | | | | | +++ |
| 1328 | 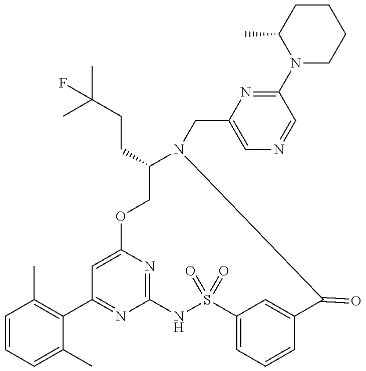 [SMILES: Cc1cccc(C)c1-c1cc2nc(n1)NS(=O)(=O)c1cccc(c1)C(=O)N(Cc1cncc(N3CCCC[C@H]3C)n1)[C@@H](CCC(C)(C)F)CO2] | | | | | | ++ |
| 1853 | 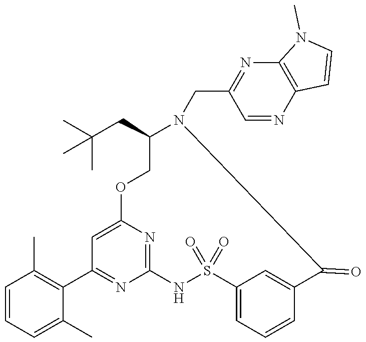 [SMILES: Cc1cccc(C)c1-c1cc2nc(n1)NS(=O)(=O)c1cccc(c1)C(=O)N(Cc1cnc3ccn(C)c3n1)[C@H](CC(C)(C)C)CO2] | | | | | | +++ |
| 1854 | 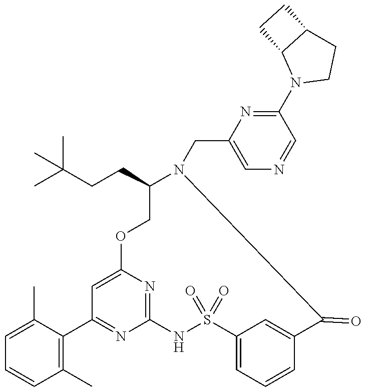 [SMILES: Cc1cccc(C)c1-c1cc2nc(n1)NS(=O)(=O)c1cccc(c1)C(=O)N(Cc1cncc(N3CC[C@H]4CC[C@H]43)n1)[C@H](CCC(C)(C)C)CO2] | | | | | | +++ |
| 1855 | 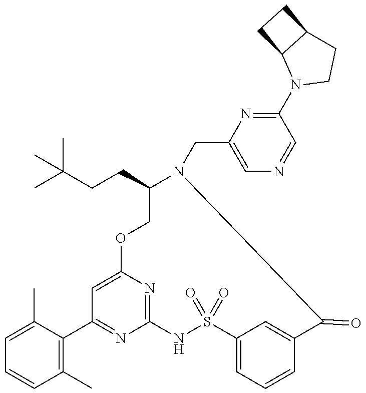 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](CCC(C)(C)C)CO2] | | | | | | +++ |
| 1856 | 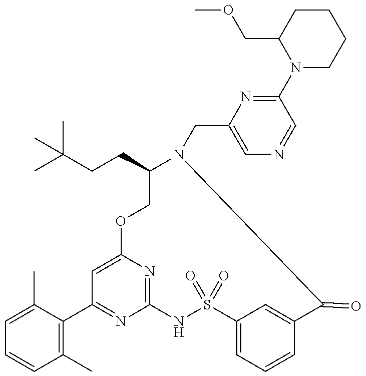 [SMILES: COCC1CCCCN1c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1857 | 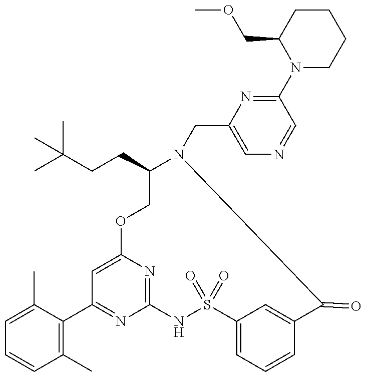 [SMILES: COC[C@H]1CCCCN1c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1858 | 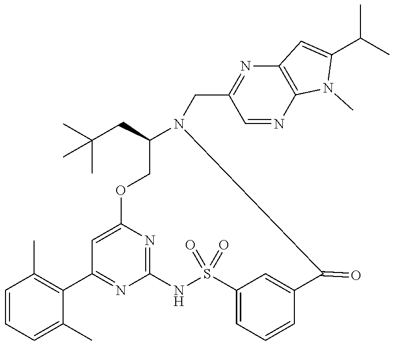 [SMILES: Cc1cccc(C)c1-c1cc2nc(n1)NS(=O)(=O)c1cccc(c1)C(=O)N(Cc1cnc3c(cc(C(C)C)n3C)n1)[C@H](CC(C)(C)C)CO2] | | | | | | +++ |
| 1859 | 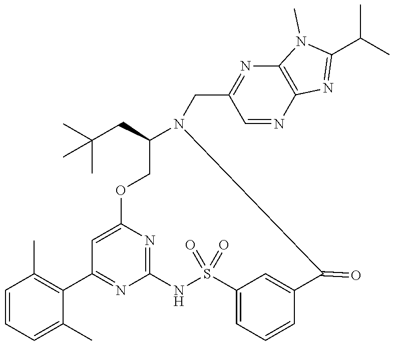 [SMILES: Cc1cccc(C)c1-c1cc2nc(n1)NS(=O)(=O)c1cccc(c1)C(=O)N(Cc1cnc3nc(C(C)C)n(C)c3n1)[C@H](CC(C)(C)C)CO2] | | | | | | +++ |
| 1329 | 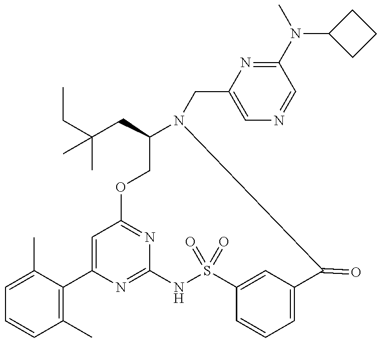 [SMILES: CCC(C)(C)C[C@@H]1COc2cc(-c3c(C)cccc3C)nc(n2)NS(=O)(=O)c2cccc(c2)C(=O)N1Cc1cncc(N(C)C2CCC2)n1] | | | | | | ++ |
| 1330 | 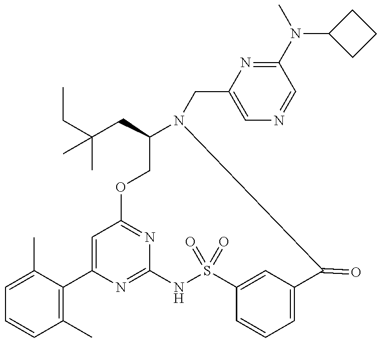 [SMILES: CCC(C)(C)C[C@@H]1COc2cc(-c3c(C)cccc3C)nc(n2)NS(=O)(=O)c2cccc(c2)C(=O)N1Cc1cncc(N(C)C2CCC2)n1] | | | | | | +++ |
| 1860 | 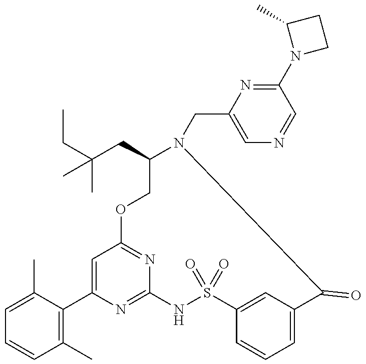 [SMILES: CCC(C)(C)C[C@@H]1COc2cc(-c3c(C)cccc3C)nc(n2)NS(=O)(=O)c2cccc(c2)C(=O)N1Cc1cncc(N2CC[C@H]2C)n1] | | | | | | ++ |
| 1861 | 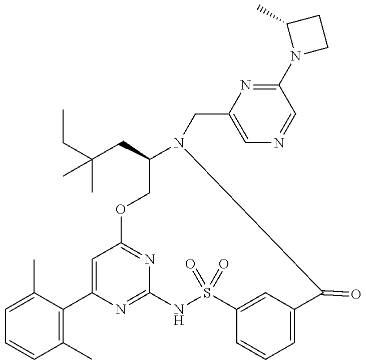 [SMILES: CCC(C)(C)C[C@@H]1COc2cc(-c3c(C)cccc3C)nc(n2)NS(=O)(=O)c2cccc(c2)C(=O)N1Cc1cncc(N2CC[C@H]2C)n1] | | | | | | +++ |
| 1862 | 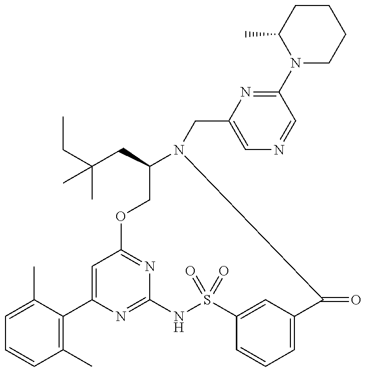 [SMILES: CCC(C)(C)C[C@@H]1COc2cc(-c3c(C)cccc3C)nc(n2)NS(=O)(=O)c2cccc(c2)C(=O)N1Cc1cncc(N2CCCC[C@H]2C)n1] | | | | | | ++ |
| 1863 | 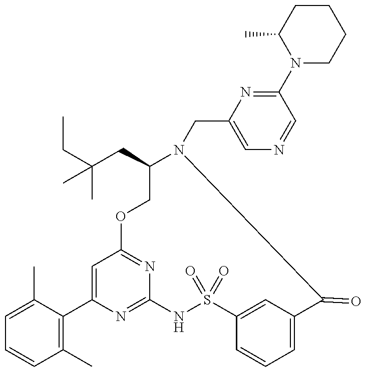 [SMILES: CCC(C)(C)C[C@@H]1COc2cc(-c3c(C)cccc3C)nc(n2)NS(=O)(=O)c2cccc(c2)C(=O)N1Cc1cncc(N2CCCC[C@H]2C)n1] | | | | | | +++ |
| 1864 | 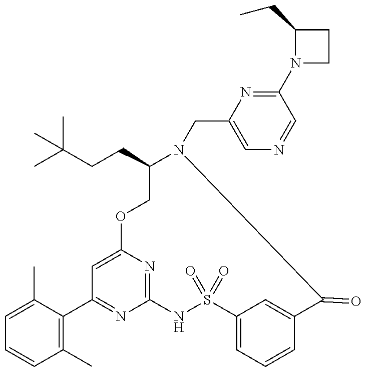 [SMILES: CC[C@H]1CCN1c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1865 | 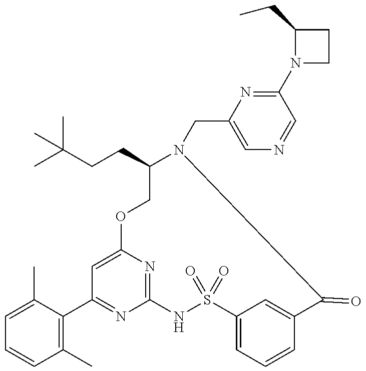 [SMILES: CC[C@H]1CCN1c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1331 | 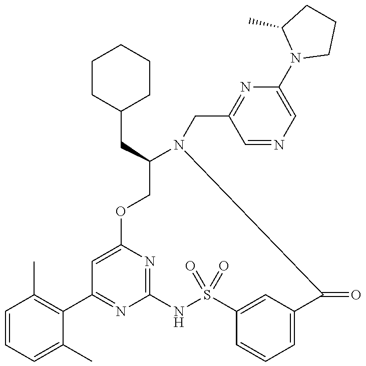 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CCCCC1)CO2] | | | | | | +++ |
| 1866 | 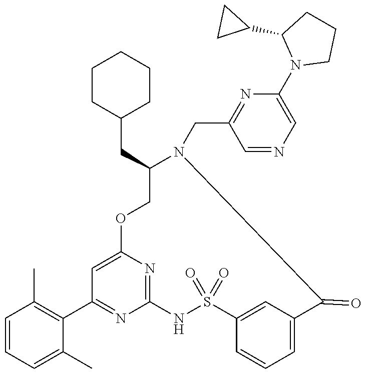 [SMILES: Cc1cccc(C)c1-c1cc2nc(n1)NS(=O)(=O)c1cccc(c1)C(=O)N(Cc1cncc(N3CCC[C@H]3C3CC3)n1)[C@H](CC1CCCCC1)CO2] | | | | | | +++ |
| 1867 | 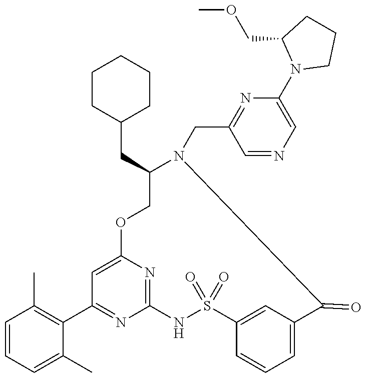 [SMILES: COC[C@@H]1CCCN1c1cncc(CN2C(=O)c3cccc(c3)S(=O)(=O)Nc3nc(cc(-c4c(C)cccc4C)n3)OC[C@H]2CC2CCCCC2)n1] | | | | | | +++ |
| 1868 | 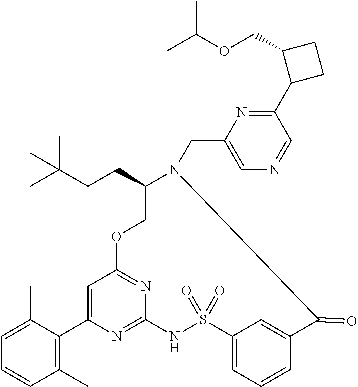 [SMILES: Cc1cccc(C)c1-c1cc2nc(n1)NS(=O)(=O)c1cccc(c1)C(=O)N(Cc1cncc(C3CC[C@H]3COC(C)C)n1)[C@H](CCC(C)(C)C)CO2] | | | | | | +++ |
| 1869 | 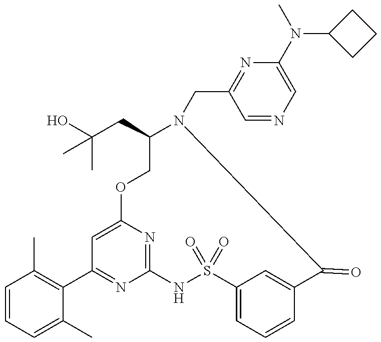 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)O)CO2] | | | | | | |
| 1332 | 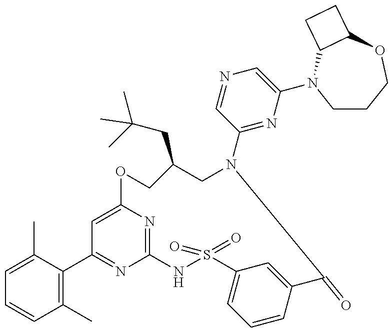 | | | | | | +++ |
| 1870 | 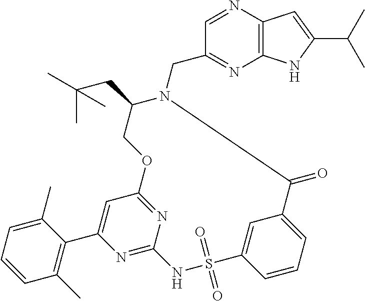 [SMILES: Cc1cccc(C)c1-c1cc2nc(n1)NS(=O)(=O)c1cccc(c1)C(=O)N(Cc1cnc3cc(C(C)C)[nH]c3n1)[C@H](CC(C)(C)C)CO2] | | | | | | |
| 1871 | 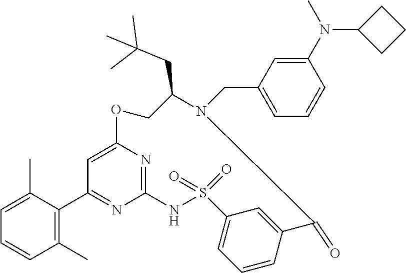 [SMILES: Cc1cccc(C)c1-c1cc2nc(n1)NS(=O)(=O)c1cccc(c1)C(=O)N(Cc1cccc(N(C)C3CCC3)c1)[C@H](CC(C)(C)C)CO2] | | | | | | |
| 1333 | 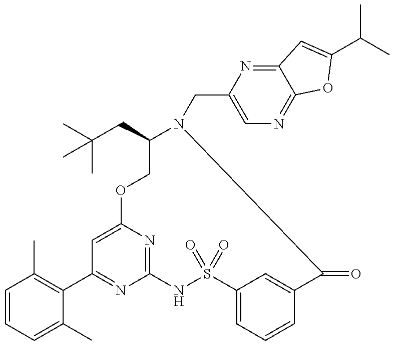 [SMILES: Cc1cccc(C)c1-c1cc2nc(n1)NS(=O)(=O)c1cccc(c1)C(=O)N(Cc1cnc3oc(C(C)C)cc3n1)[C@H](CC(C)(C)C)CO2] | | | | | | +++ |
| 1334 | 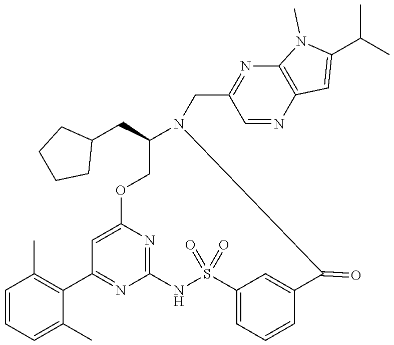 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCC1)CO2] | | | | | | +++ |
| 1872 | 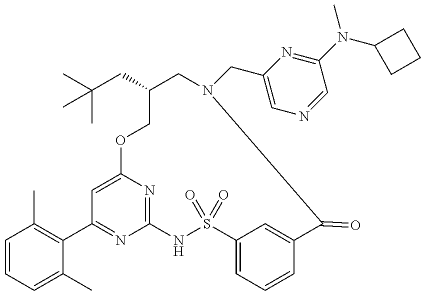 [SMILES: Cc1cccc(C)c1-c1cc2nc(n1)NS(=O)(=O)c1cccc(c1)C(=O)N(Cc1cncc(N(C)C3CCC3)n1)C[C@@H](CC(C)(C)C)CO2] | | | | | | |
| 1873 | 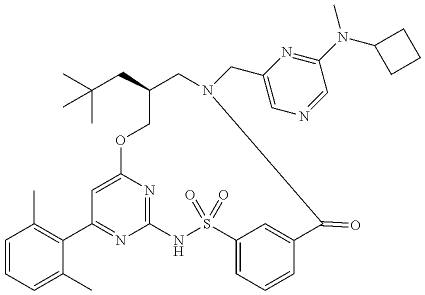 [SMILES: Cc1cccc(C)c1-c1cc2nc(n1)NS(=O)(=O)c1cccc(c1)C(=O)N(Cc1cncc(N(C)C3CCC3)n1)C[C@H](CC(C)(C)C)CO2] | | | | | | |
| 1874 | 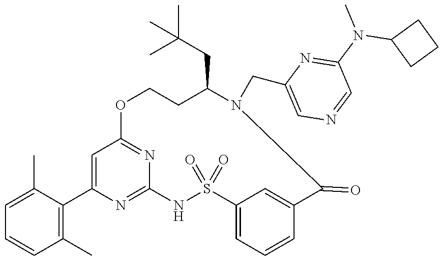 [SMILES: Cc1cccc(C)c1-c1cc2nc(n1)NS(=O)(=O)c1cccc(c1)C(=O)N(Cc1cncc(N(C)C3CCC3)n1)[C@H](CC(C)(C)C)CCO2] | | | | | | |
| 1875 | 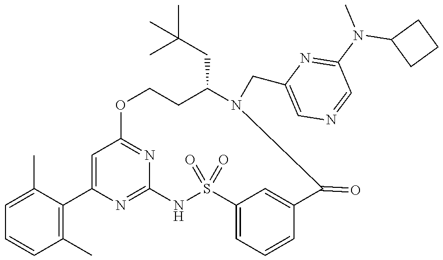 [SMILES: Cc1cccc(C)c1-c1cc2nc(n1)NS(=O)(=O)c1cccc(c1)C(=O)N(Cc1cncc(N(C)C3CCC3)n1)[C@@H](CC(C)(C)C)CCO2] | | | | | | |
| 1876 | 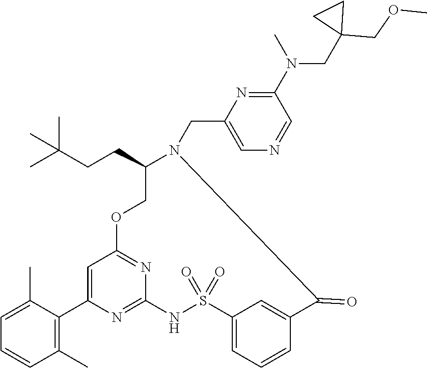 [SMILES: COCC1(CN(C)c2cncc(CN3C(=O)c4cccc(c4)S(=O)(=O)Nc4nc(cc(-c5c(C)cccc5C)n4)OC[C@H]3CCC(C)(C)C)n2)CC1] | | | | | | +++ |
| 1877 | 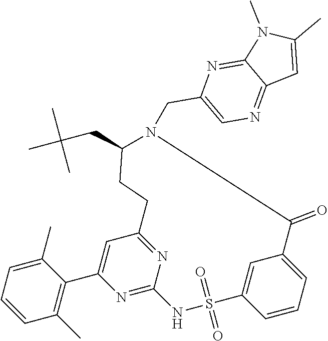 [SMILES: Cc1cccc(C)c1-c1cc2nc(n1)NS(=O)(=O)c1cccc(c1)C(=O)N(Cc1cnc3cc(C)n(C)c3n1)[C@H](CC(C)(C)C)CC2] | | | | | | +++ |
| 1878 | 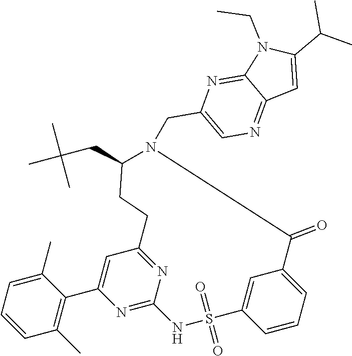 [SMILES: CCn1c(C(C)C)cc2ncc(CN3C(=O)c4cccc(c4)S(=O)(=O)Nc4nc(cc(-c5c(C)cccc5C)n4)CC[C@H]3CC(C)(C)C)nc21] | | | | | | +++ |
| 1879 | 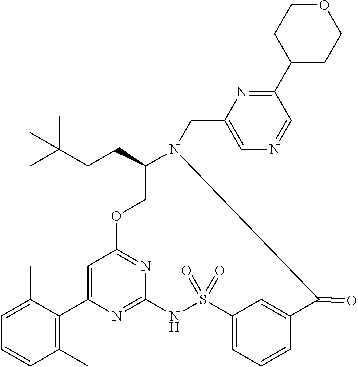 [SMILES: Cc1cccc(C)c1-c1cc2nc(n1)NS(=O)(=O)c1cccc(c1)C(=O)N(Cc1cncc(C3CCOCC3)n1)[C@H](CCC(C)(C)C)CO2] | | | | | | +++ |
| 1880 | 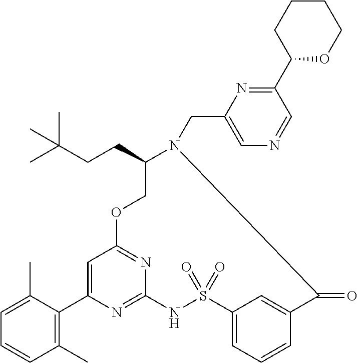 [SMILES: Cc1cccc(C)c1-c1cc2nc(n1)NS(=O)(=O)c1cccc(c1)C(=O)N(Cc1cncc([C@@H]3CCCCO3)n1)[C@H](CCC(C)(C)C)CO2] | | | | | | +++ |
| 1881 | 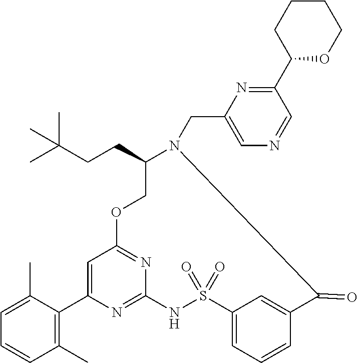 [SMILES: Cc1cccc(C)c1-c1cc2nc(n1)NS(=O)(=O)c1cccc(c1)C(=O)N(Cc1cncc([C@@H]3CCCCO3)n1)[C@H](CCC(C)(C)C)CO2] | | | | | | +++ |
| 1335 | 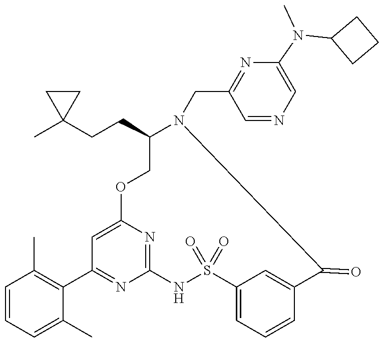 [SMILES: Cc1cccc(C)c1-c1cc2nc(n1)NS(=O)(=O)c1cccc(c1)C(=O)N(Cc1cncc(N(C)C3CCC3)n1)[C@H](CCC1(C)CC1)CO2] | | | | | | |
| 1336 | 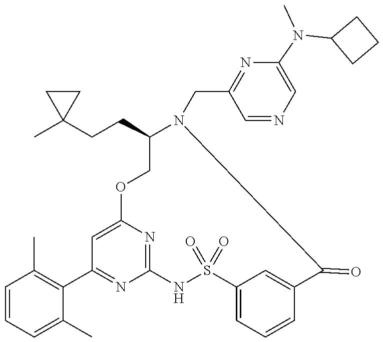 [SMILES: Cc1cccc(C)c1-c1cc2nc(n1)NS(=O)(=O)c1cccc(c1)C(=O)N(Cc1cncc(N(C)C3CCC3)n1)[C@H](CCC1(C)CC1)CO2] | | | | | | +++ |
| 1882 | 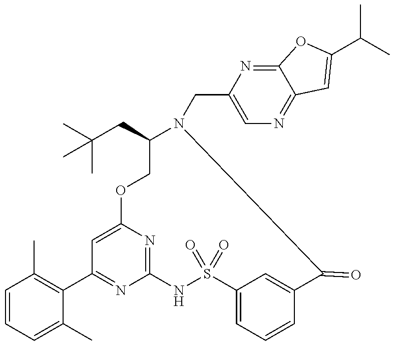 [SMILES: Cc1cccc(C)c1-c1cc2nc(n1)NS(=O)(=O)c1cccc(c1)C(=O)N(Cc1cnc3cc(C(C)C)oc3n1)[C@H](CC(C)(C)C)CO2] | | | | | | +++ |
| 1883 | 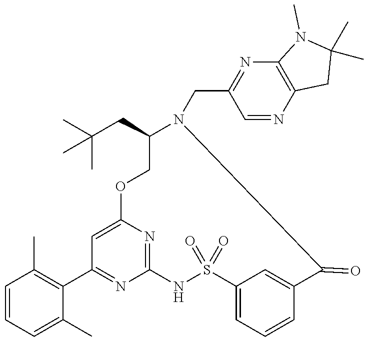 [SMILES: Cc1cccc(C)c1-c1cc2nc(n1)NS(=O)(=O)c1cccc(c1)C(=O)N(Cc1cnc3c(n1)N(C)C(C)(C)C3)[C@H](CC(C)(C)C)CO2] | | | | | | +++ |
| 1884 | 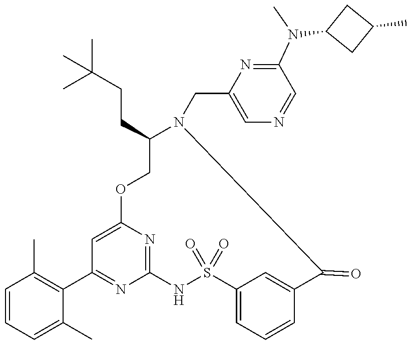 [SMILES: Cc1cccc(C)c1-c1cc2nc(n1)NS(=O)(=O)c1cccc(c1)C(=O)N(Cc1cncc(N(C)[C@H]3C[C@@H](C)C3)n1)[C@H](CCC(C)(C)C)CO2] | | | | | | +++ |
| 1885 | 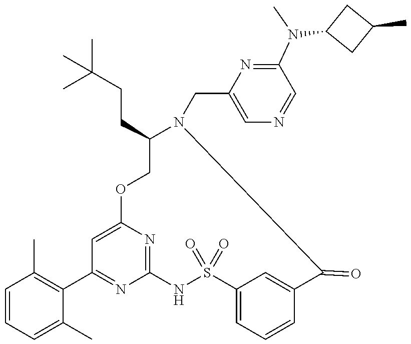 [SMILES: Cc1cccc(C)c1-c1cc2nc(n1)NS(=O)(=O)c1cccc(c1)C(=O)N(Cc1cncc(N(C)[C@H]3C[C@H](C)C3)n1)[C@H](CCC(C)(C)C)CO2] | | | | | | +++ |
| 1886 | 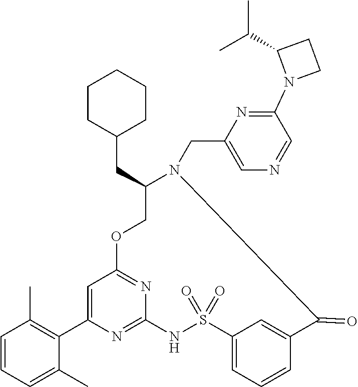 [SMILES: Cc1cccc(C)c1-c1cc2nc(n1)NS(=O)(=O)c1cccc(c1)C(=O)N(Cc1cncc(N3CC[C@H]3C(C)C)n1)[C@H](CC1CCCCC1)CO2] | | | | | | |
| 1887 | 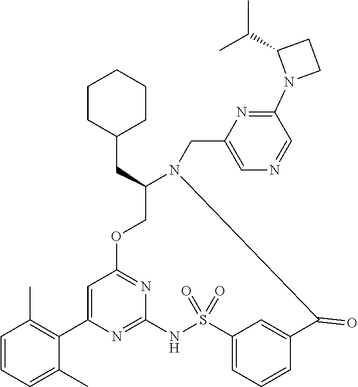 [SMILES: Cc1cccc(C)c1-c1cc2nc(n1)NS(=O)(=O)c1cccc(c1)C(=O)N(Cc1cncc(N3CC[C@H]3C(C)C)n1)[C@H](CC1CCCCC1)CO2] | | | | | | +++ |
| 1888 | 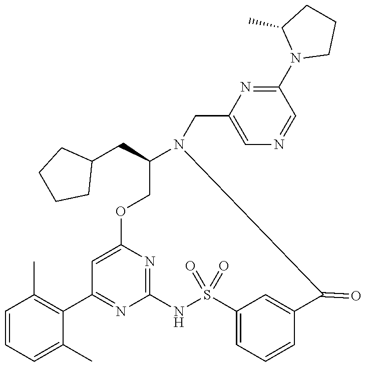 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CCCC1)CO2] | | | | | | +++ |
| 1889 | 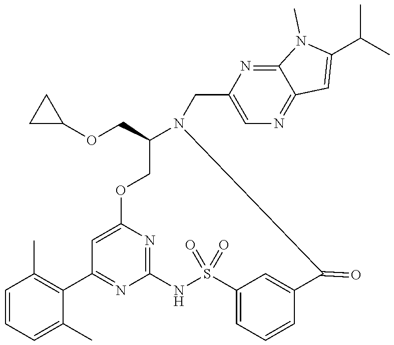 [SMILES: Cc1cccc(C)c1-c1cc2nc(n1)NS(=O)(=O)c1cccc(c1)C(=O)N(Cc1cnc3cc(C(C)C)n(C)c3n1)[C@H](COC1CC1)CO2] | | | | | | +++ |
| 1890 | 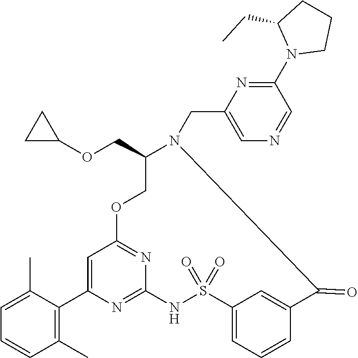 [SMILES: CC[C@@H]1CCCN1c1cncc(CN2C(=O)c3cccc(c3)S(=O)(=O)Nc3nc(cc(-c4c(C)cccc4C)n3)OC[C@H]2COC2CC2)n1] | | | | | | +++ |
| 1891 | 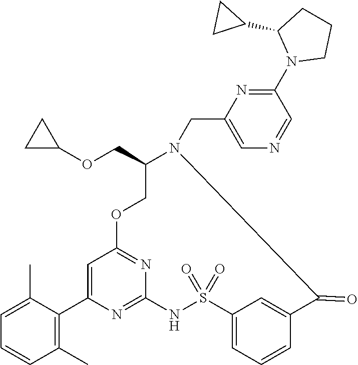 [SMILES: Cc1cccc(C)c1-c1cc2nc(n1)NS(=O)(=O)c1cccc(c1)C(=O)N(Cc1cncc(N3CCC[C@H]3C3CC3)n1)[C@H](COC1CC1)CO2] | | | | | | +++ |
| 1892 | 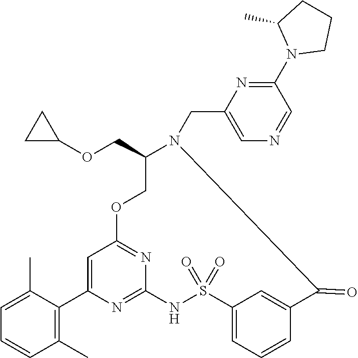 [SMILES: Cc1cccc(C)c1-c1cc2nc(n1)NS(=O)(=O)c1cccc(c1)C(=O)N(Cc1cncc(N3CCC[C@H]3C)n1)[C@H](COC1CC1)CO2] | | | | | | +++ |
| 1893 | 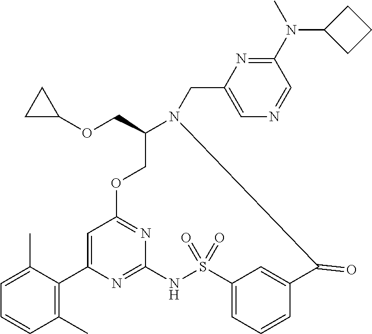 [SMILES: Cc1cccc(C)c1-c1cc2nc(n1)NS(=O)(=O)c1cccc(c1)C(=O)N(Cc1cncc(N(C)C3CCC3)n1)[C@H](COC1CC1)CO2] | | | | | | +++ |
| 1894 | 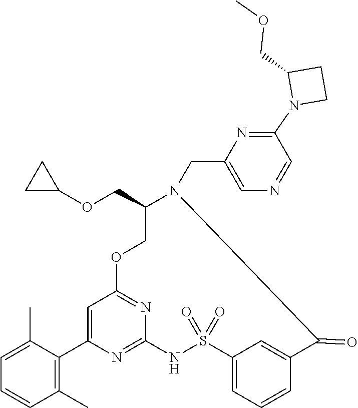 [SMILES: COC[C@@H]1CCN1c1cncc(CN2C(=O)c3cccc(c3)S(=O)(=O)Nc3nc(cc(-c4c(C)cccc4C)n3)OC[C@H]2COC2CC2)n1] | | | | | | +++ |
| 1895 | 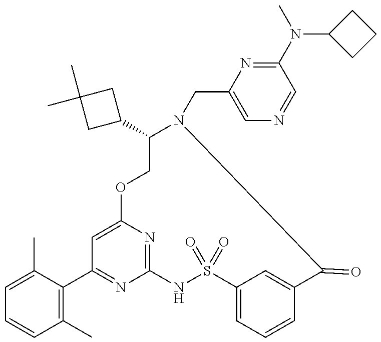 [SMILES: Cc1cccc(C)c1-c1cc2nc(n1)NS(=O)(=O)c1cccc(c1)C(=O)N(Cc1cncc(N(C)C3CCC3)n1)[C@@H](C1CC(C)(C)C1)CO2] | | | | | | ++ |
| 1896 | 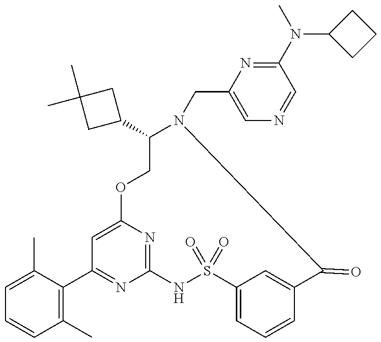 [SMILES: Cc1cccc(C)c1-c1cc2nc(n1)NS(=O)(=O)c1cccc(c1)C(=O)N(Cc1cncc(N(C)C3CCC3)n1)[C@@H](C1CC(C)(C)C1)CO2] | | | | | | +++ |
| 1337 | 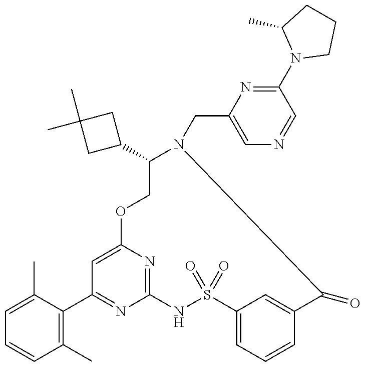 [SMILES: Cc1cccc(C)c1-c1cc2nc(n1)NS(=O)(=O)c1cccc(c1)C(=O)N(Cc1cncc(N3CCC[C@H]3C)n1)[C@@H](C1CC(C)(C)C1)CO2] | | | | | | ++ |
| 1338 | 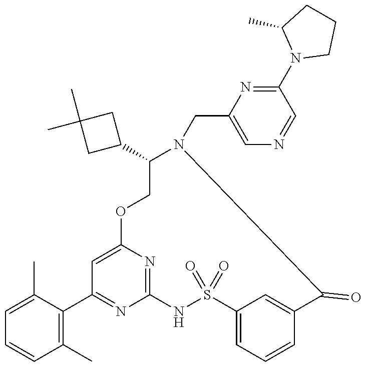 [SMILES: Cc1cccc(C)c1-c1cc2nc(n1)NS(=O)(=O)c1cccc(c1)C(=O)N(Cc1cncc(N3CCC[C@H]3C)n1)[C@@H](C1CC(C)(C)C1)CO2] | | | | | | +++ |
| 1897 | 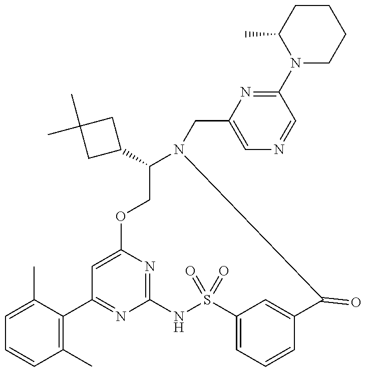 [SMILES: Cc1cccc(C)c1-c1cc2nc(n1)NS(=O)(=O)c1cccc(c1)C(=O)N(Cc1cncc(N3CCCC[C@H]3C)n1)[C@@H](C1CC(C)(C)C1)CO2] | | | | | | + |
| 1898 | 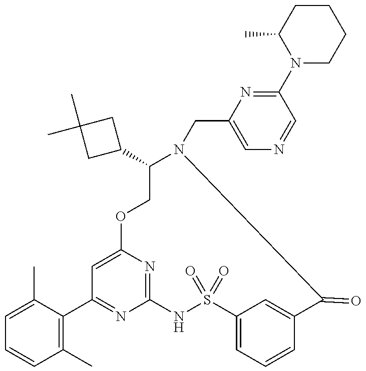 [SMILES: Cc1cccc(C)c1-c1cc2nc(n1)NS(=O)(=O)c1cccc(c1)C(=O)N(Cc1cncc(N3CCCC[C@H]3C)n1)[C@@H](C1CC(C)(C)C1)CO2] | | | | | | +++ |
| 1339 | 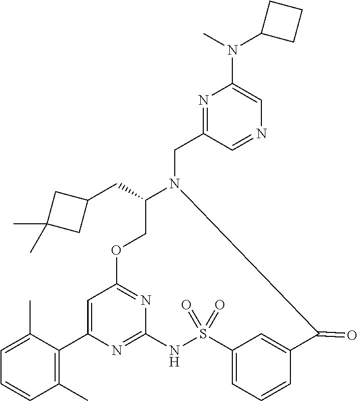 [SMILES: Cc1cccc(C)c1-c1cc2nc(n1)NS(=O)(=O)c1cccc(c1)C(=O)N(Cc1cncc(N(C)C3CCC3)n1)[C@@H](CC1CC(C)(C)C1)CO2] | | | | | | +++ |
| 1340 | 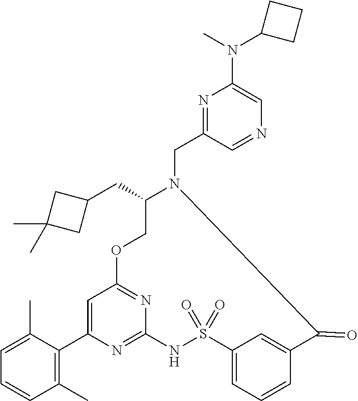 [SMILES: Cc1cccc(C)c1-c1cc2nc(n1)NS(=O)(=O)c1cccc(c1)C(=O)N(Cc1cncc(N(C)C3CCC3)n1)[C@@H](CC1CC(C)(C)C1)CO2] | | | | | | ++ |
| 1899 | 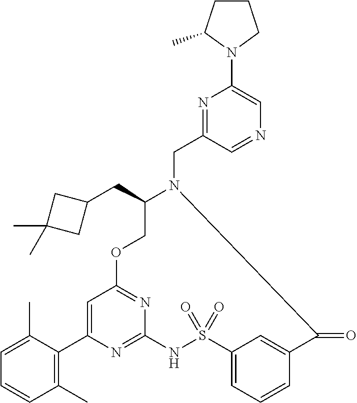 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CC(C)(C)C1)CO2] | | | | | | +++ |
| 1900 | 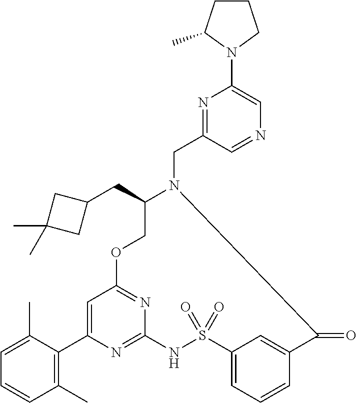 [SMILES: Cc1cccc(C)c1-c1cc2nc(n1)NS(=O)(=O)c1cccc(c1)C(=O)N(Cc1cncc(N3CCC[C@H]3C)n1)[C@H](CC1CC(C)(C)C1)CO2] | | | | | | + |
| 1901 | 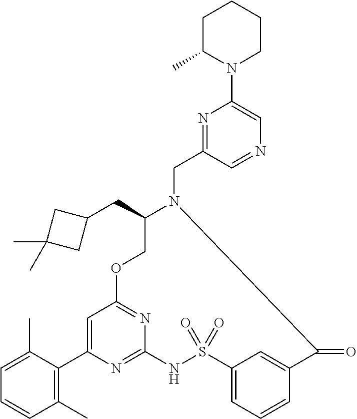 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CC(C)(C)C1)CO2] | | | | | | +++ |
| 1902 | 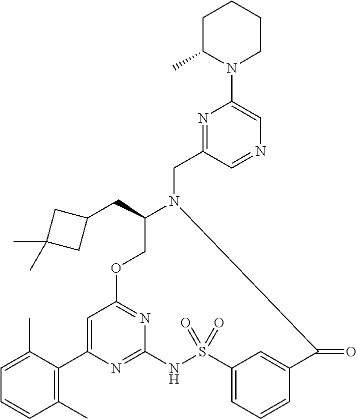 [SMILES: Cc1cccc(C)c1-c1cc2nc(n1)NS(=O)(=O)c1cccc(c1)C(=O)N(Cc1cncc(N3CCCC[C@H]3C)n1)[C@H](CC1CC(C)(C)C1)CO2] | | | | | | +++ |
| 1903 | 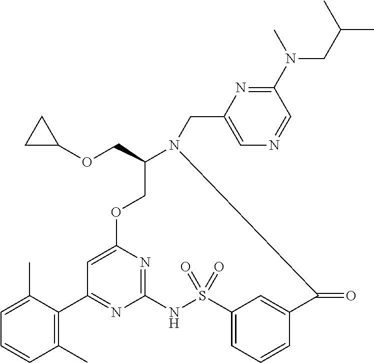 [SMILES: Cc1cccc(C)c1-c1cc2nc(n1)NS(=O)(=O)c1cccc(c1)C(=O)N(Cc1cncc(N(C)CC(C)C)n1)[C@H](COC1CC1)CO2] | | | | | | +++ |
| 1904 | 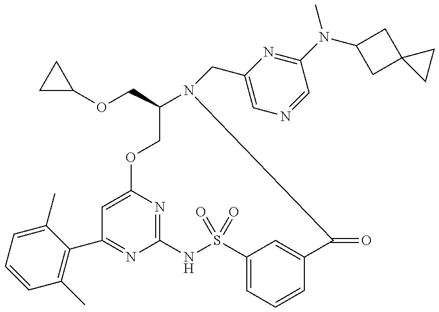 [SMILES: Cc1cccc(C)c1-c1cc2nc(n1)NS(=O)(=O)c1cccc(c1)C(=O)N(Cc1cncc(N(C)C3CC4(CC4)C3)n1)[C@H](COC1CC1)CO2] | | | | | | +++ |
| 1905 | 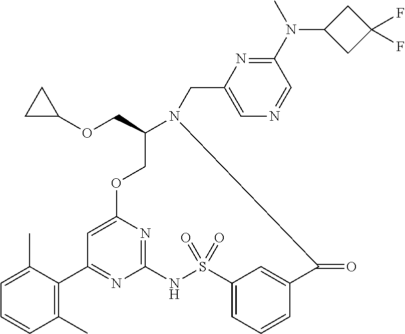 [SMILES: Cc1cccc(C)c1-c1cc2nc(n1)NS(=O)(=O)c1cccc(c1)C(=O)N(Cc1cncc(N(C)C3CC(F)(F)C3)n1)[C@H](COC1CC1)CO2] | | | | | | +++ |
| 1341 | 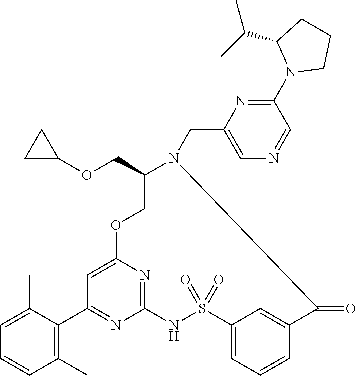 [SMILES: Cc1cccc(C)c1-c1cc2nc(n1)NS(=O)(=O)c1cccc(c1)C(=O)N(Cc1cncc(N3CCC[C@H]3C(C)C)n1)[C@H](COC1CC1)CO2] | | | | | | +++ |
| 1906 | 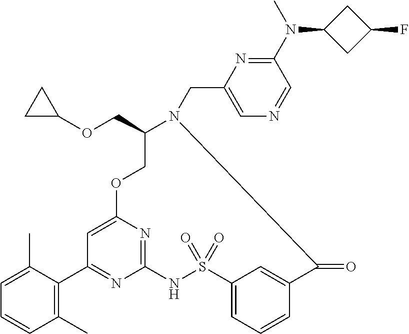 [SMILES: Cc1cccc(C)c1-c1cc2nc(n1)NS(=O)(=O)c1cccc(c1)C(=O)N(Cc1cncc(N(C)[C@H]3C[C@@H](F)C3)n1)[C@H](COC1CC1)CO2] | | | | | | +++ |
| 1907 | 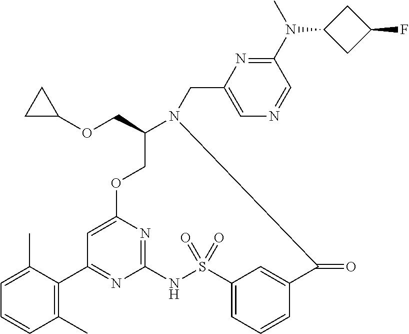 [SMILES: Cc1cccc(C)c1-c1cc2nc(n1)NS(=O)(=O)c1cccc(c1)C(=O)N(Cc1cncc(N(C)[C@H]3C[C@H](F)C3)n1)[C@H](COC1CC1)CO2] | | | | | | +++ |
| 1908 | 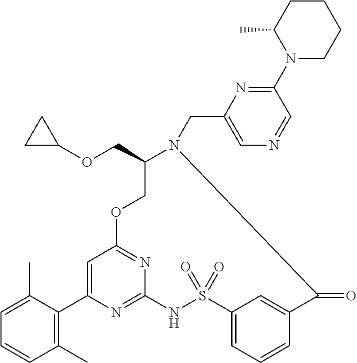 [SMILES: Cc1cccc(C)c1-c1cc2nc(n1)NS(=O)(=O)c1cccc(c1)C(=O)N(Cc1cncc(N3CCCC[C@H]3C)n1)[C@H](COC1CC1)CO2] | | | | | | +++ |
| 1909 | 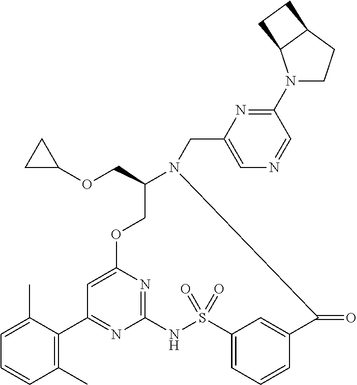 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](COC1CC1)CO2] | | | | | | +++ |
| 1910 | 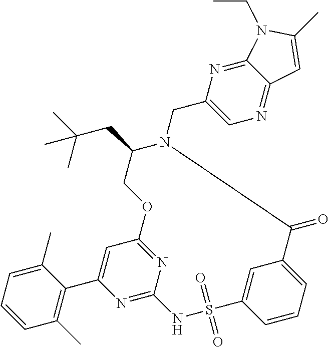 [SMILES: CCn1c(C)cc2ncc(CN3C(=O)c4cccc(c4)S(=O)(=O)Nc4nc(cc(-c5c(C)cccc5C)n4)OC[C@H]3CC(C)(C)C)nc21] | | | | | | +++ |
| 1911 | 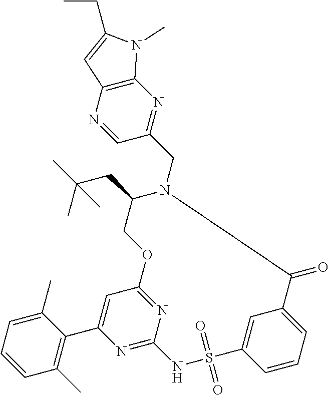 [SMILES: CCc1cc2ncc(CN3C(=O)c4cccc(c4)S(=O)(=O)Nc4nc(cc(-c5c(C)cccc5C)n4)OC[C@H]3CC(C)(C)C)nc2n1C] | | | | | | +++ |
| 1912 | 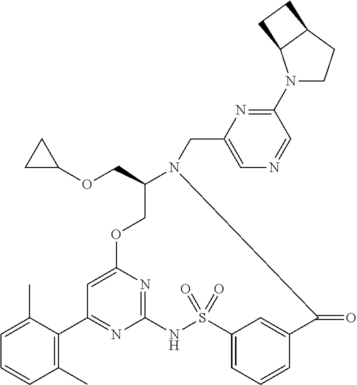 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](COC1CC1)CO2] | | | | | | +++ |
| 1913 | 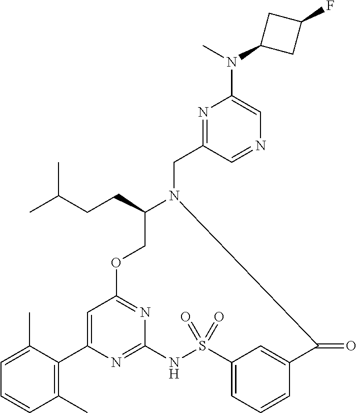 [SMILES: Cc1cccc(C)c1-c1cc2nc(n1)NS(=O)(=O)c1cccc(c1)C(=O)N(Cc1cncc(N(C)[C@H]3C[C@@H](F)C3)n1)[C@H](CCC(C)C)CO2] | | | | | | +++ |
| 1914 | 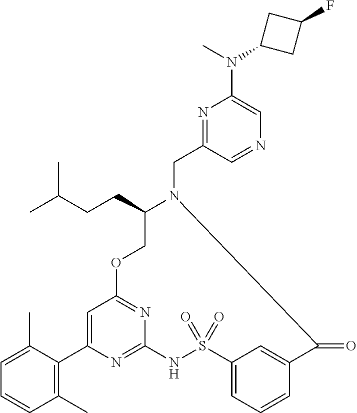 [SMILES: Cc1cccc(C)c1-c1cc2nc(n1)NS(=O)(=O)c1cccc(c1)C(=O)N(Cc1cncc(N(C)[C@H]3C[C@H](F)C3)n1)[C@H](CCC(C)C)CO2] | | | | | | +++ |
| 1915 | 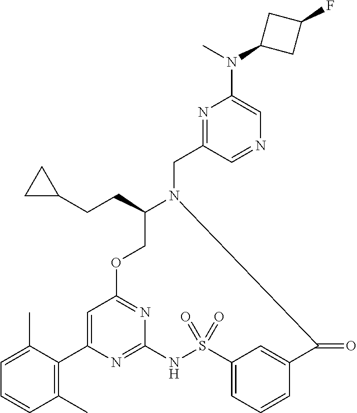 [SMILES: Cc1cccc(C)c1-c1cc2nc(n1)NS(=O)(=O)c1cccc(c1)C(=O)N(Cc1cncc(N(C)[C@H]3C[C@@H](F)C3)n1)[C@H](CCC1CC1)CO2] | | | | | | +++ |
| 1916 | 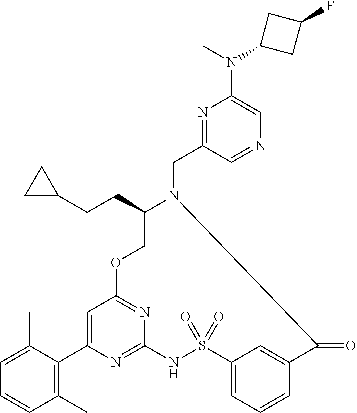 [SMILES: Cc1cccc(C)c1-c1cc2nc(n1)NS(=O)(=O)c1cccc(c1)C(=O)N(Cc1cncc(N(C)[C@H]3C[C@H](F)C3)n1)[C@H](CCC1CC1)CO2] | | | | | | +++ |
| 1917 | 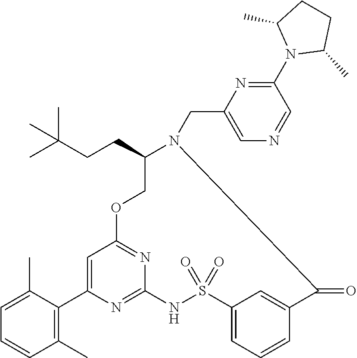 [SMILES: Cc1cccc(C)c1-c1cc2nc(n1)NS(=O)(=O)c1cccc(c1)C(=O)N(Cc1cncc(N3[C@H](C)CC[C@@H]3C)n1)[C@H](CCC(C)(C)C)CO2] | | | | | | +++ |
| 1918 | 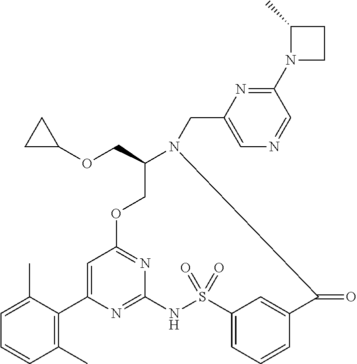 [SMILES: Cc1cccc(C)c1-c1cc2nc(n1)NS(=O)(=O)c1cccc(c1)C(=O)N(Cc1cncc(N3CC[C@H]3C)n1)[C@H](COC1CC1)CO2] | | | | | | +++ |
| 1919 | 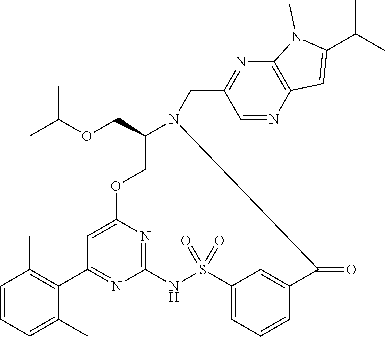 [SMILES: Cc1cccc(C)c1-c1cc2nc(n1)NS(=O)(=O)c1cccc(c1)C(=O)N(Cc1cnc3cc(C(C)C)n(C)c3n1)[C@H](COC(C)C)CO2] | | | | | | +++ |
| 1920 | 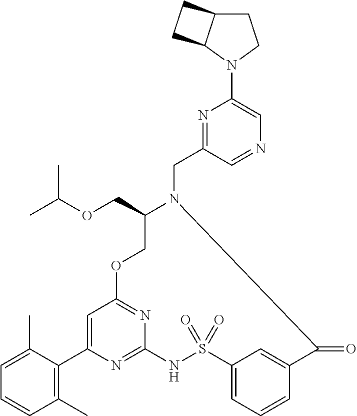 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](COC(C)C)CO2] | | | | | | +++ |
| 1921 | 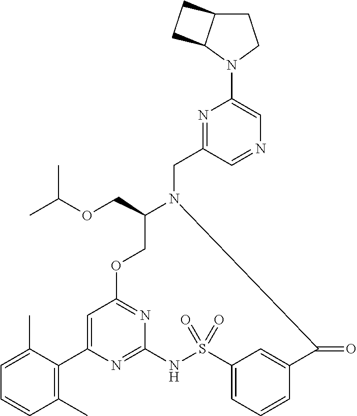 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](COC(C)C)CO2] | | | | | | +++ |
| 1922 | 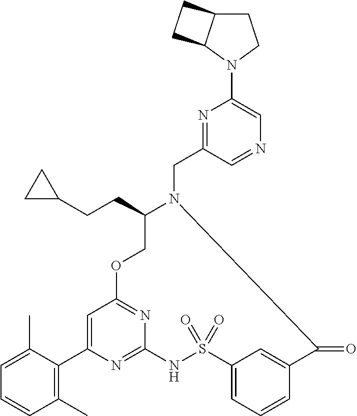 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](CCC1CC1)CO2] | | | | | | +++ |
| 1923 | 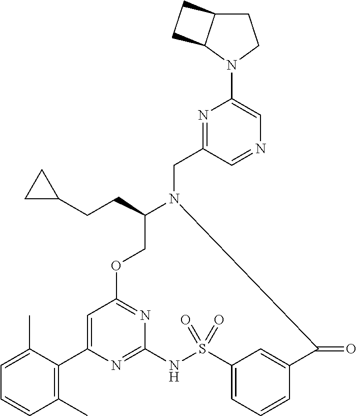 [SMILES: Cc1cccc(C)c1-c1cc2nc(n1)NS(=O)(=O)c1cccc(c1)C(=O)N(Cc1cncc(N3CC[C@@H]4CC[C@@H]43)n1)[C@H](CCC1CC1)CO2] | | | | | | +++ |
| 1924 | 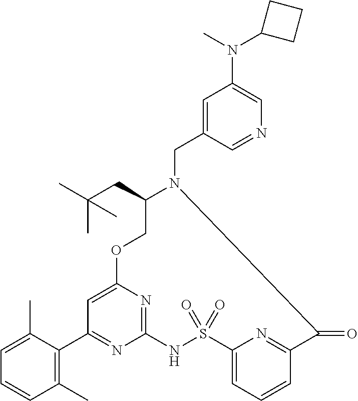 [SMILES: Cc1cccc(C)c1-c1cc2nc(n1)NS(=O)(=O)c1cccc(n1)C(=O)N(Cc1cncc(N(C)C3CCC3)c1)[C@H](CC(C)(C)C)CO2] | | | | | | +++ |
| 1925 | 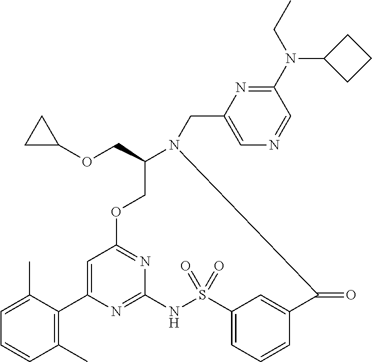 [SMILES: CCN(c1cncc(CN2C(=O)c3cccc(c3)S(=O)(=O)Nc3nc(cc(-c4c(C)cccc4C)n3)OC[C@H]2COC2CC2)n1)C1CCC1] | | | | | | +++ |
| 1926 | 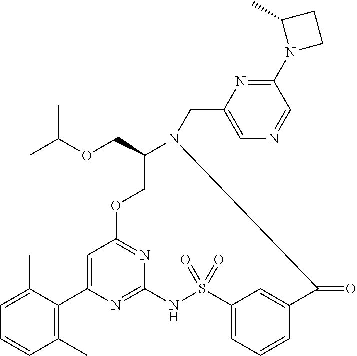 [SMILES: Cc1cccc(C)c1-c1cc2nc(n1)NS(=O)(=O)c1cccc(c1)C(=O)N(Cc1cncc(N3CC[C@H]3C)n1)[C@H](COC(C)C)CO2] | | | | | | +++ |
| 1927 | 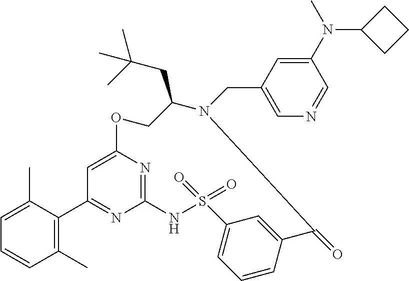 [SMILES: Cc1cccc(C)c1-c1cc2nc(n1)NS(=O)(=O)c1cccc(c1)C(=O)N(Cc1cncc(N(C)C3CCC3)c1)[C@H](CC(C)(C)C)CO2] | | | | | | +++ |
| 1928 | 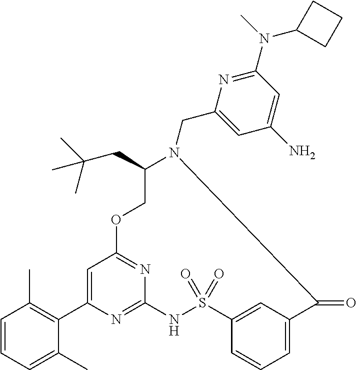 [SMILES: Cc1cccc(C)c1-c1cc2nc(n1)NS(=O)(=O)c1cccc(c1)C(=O)N(Cc1cc(N)cc(N(C)C3CCC3)n1)[C@H](CC(C)(C)C)CO2] | | | | | | +++ |
| 1929 | 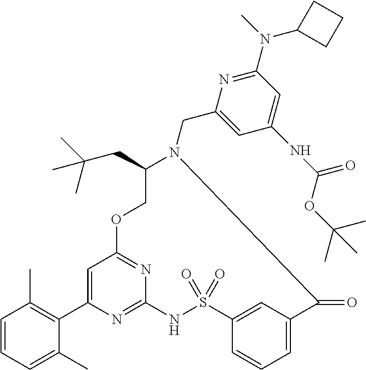 [SMILES: Cc1cccc(C)c1-c1cc2nc(n1)NS(=O)(=O)c1cccc(c1)C(=O)N(Cc1cc(NC(=O)OC(C)(C)C)cc(N(C)C3CCC3)n1)[C@H](CC(C)(C)C)CO2] | | | | | | +++ |
| 1930 | 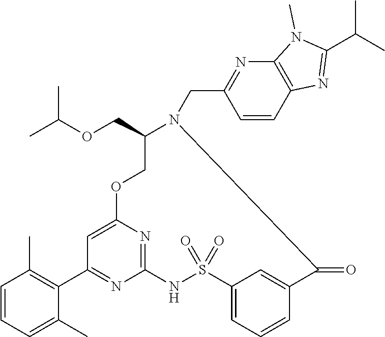 [SMILES: Cc1cccc(C)c1-c1cc2nc(n1)NS(=O)(=O)c1cccc(c1)C(=O)N(Cc1ccc3nc(C(C)C)n(C)c3n1)[C@H](COC(C)C)CO2] | | | | | | +++ |
| 1931 | 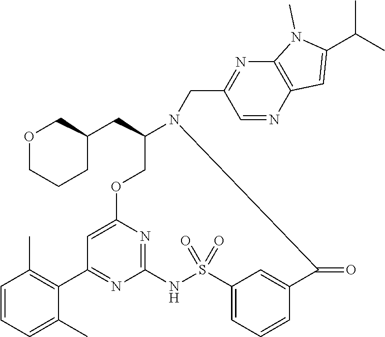 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@H]1CCCOC1)CO2] | | | | | | +++ |
| 1932 | 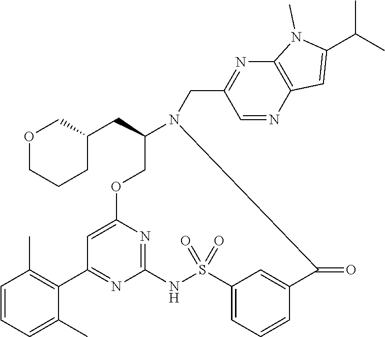 [SMILES: Cc1cccc(C)c1-c1cc2nc(n1)NS(=O)(=O)c1cccc(c1)C(=O)N(Cc1cnc3cc(C(C)C)n(C)c3n1)[C@H](C[C@@H]1CCCOC1)CO2] | | | | | | +++ |
| 1933 | 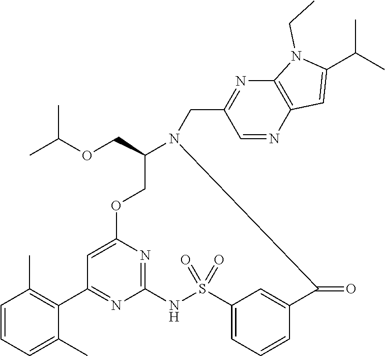 [SMILES: CCn1c(C(C)C)cc2ncc(CN3C(=O)c4cccc(c4)S(=O)(=O)Nc4nc(cc(-c5c(C)cccc5C)n4)OC[C@H]3COC(C)C)nc21] | | | | | | +++ |
| 1934 | 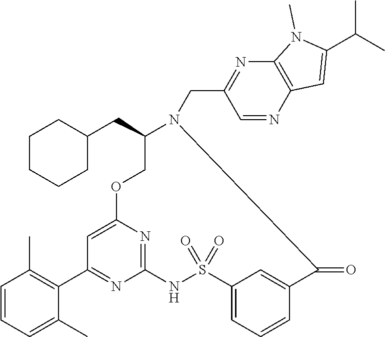 [SMILES: Cc1cccc(C)c1-c1cc2nc(n1)NS(=O)(=O)c1cccc(c1)C(=O)N(Cc1cnc3cc(C(C)C)n(C)c3n1)[C@H](CC1CCCCC1)CO2] | | | | | | +++ |
| 1935 | 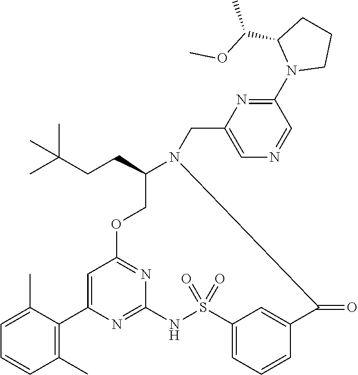 [SMILES: CO[C@H](C)[C@@H]1CCCN1c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1936 | 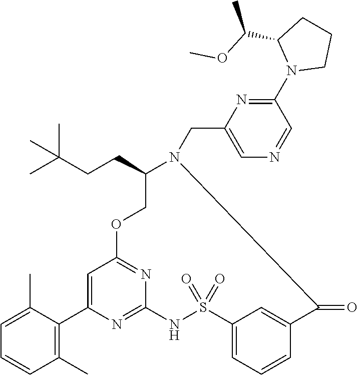 [SMILES: CO[C@@H](C)[C@@H]1CCCN1c1cncc(CN2C(=O)c3cccc(c3)S(=O)(=O)Nc3nc(cc(-c4c(C)cccc4C)n3)OC[C@H]2CCC(C)(C)C)n1] | | | | | | +++ |
| 1937 | 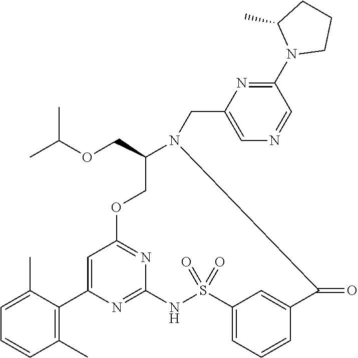 [SMILES: Cc1cccc(C)c1-c1cc2nc(n1)NS(=O)(=O)c1cccc(c1)C(=O)N(Cc1cncc(N3CCC[C@H]3C)n1)[C@H](COC(C)C)CO2] | | | | | | +++ |
| 1938 | 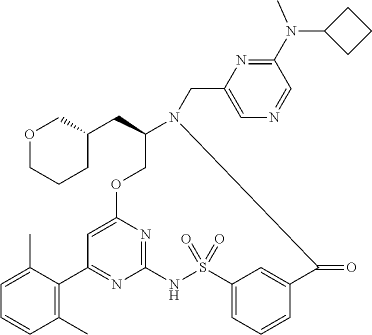 [SMILES: Cc1cccc(C)c1-c1cc2nc(n1)NS(=O)(=O)c1cccc(c1)C(=O)N(Cc1cncc(N(C)C3CCC3)n1)[C@H](C[C@@H]1CCCOC1)CO2] | | | | | | +++ |
| 1939 | 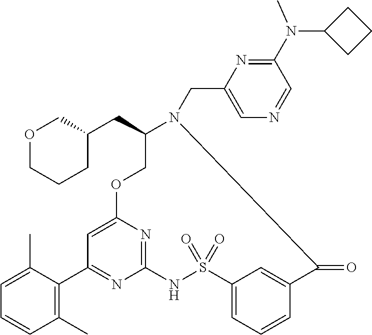 [SMILES: Cc1cccc(C)c1-c1cc2nc(n1)NS(=O)(=O)c1cccc(c1)C(=O)N(Cc1cncc(N(C)C3CCC3)n1)[C@H](C[C@@H]1CCCOC1)CO2] | | | | | | +++ |
| 1940 | 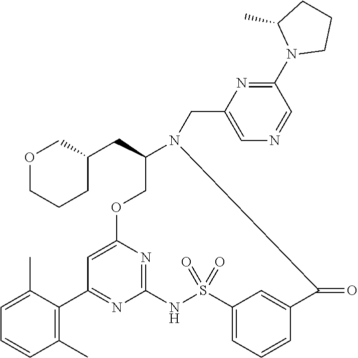 [SMILES: Cc1cccc(C)c1-c1cc2nc(n1)NS(=O)(=O)c1cccc(c1)C(=O)N(Cc1cncc(N3CCC[C@H]3C)n1)[C@H](C[C@@H]1CCCOC1)CO2] | | | | | | +++ |
| 1941 | 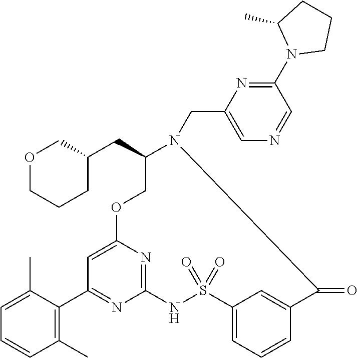 [SMILES: Cc1cccc(C)c1-c1cc2nc(n1)NS(=O)(=O)c1cccc(c1)C(=O)N(Cc1cncc(N3CCC[C@H]3C)n1)[C@H](C[C@@H]1CCCOC1)CO2] | | | | | | +++ |
| 1942 | 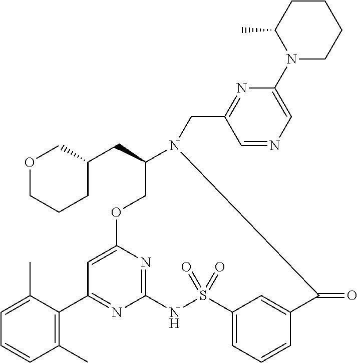 [SMILES: Cc1cccc(C)c1-c1cc2nc(n1)NS(=O)(=O)c1cccc(c1)C(=O)N(Cc1cncc(N3CCCC[C@H]3C)n1)[C@H](C[C@@H]1CCCOC1)CO2] | | | | | | +++ |
| 1943 | 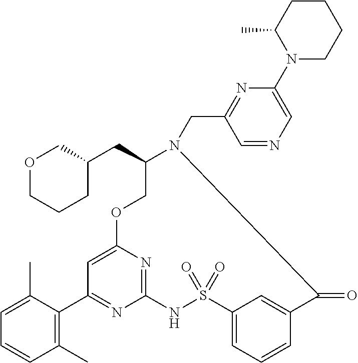 [SMILES: Cc1cccc(C)c1-c1cc2nc(n1)NS(=O)(=O)c1cccc(c1)C(=O)N(Cc1cncc(N3CCCC[C@H]3C)n1)[C@H](C[C@@H]1CCCOC1)CO2] | | | | | | +++ |
| 1944 | 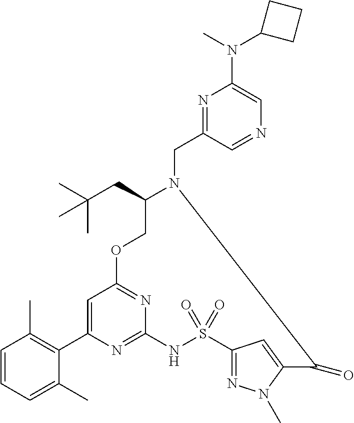 [SMILES: Cc1cccc(C)c1-c1cc2nc(n1)NS(=O)(=O)c1cc(n(C)n1)C(=O)N(Cc1cncc(N(C)C3CCC3)n1)[C@H](CC(C)(C)C)CO2] | | | | | | +++ |
| 1945 | 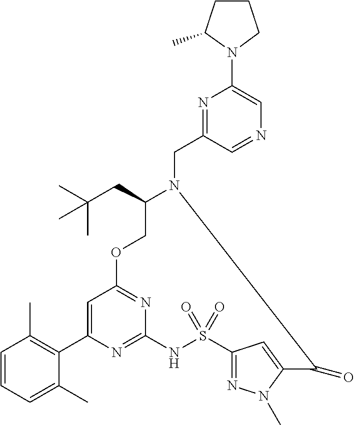 [SMILES: Cc1cccc(C)c1-c1cc2nc(n1)NS(=O)(=O)c1cc(n(C)n1)C(=O)N(Cc1cncc(N3CCC[C@H]3C)n1)[C@H](CC(C)(C)C)CO2] | | | | | | +++ |
| 1946 | 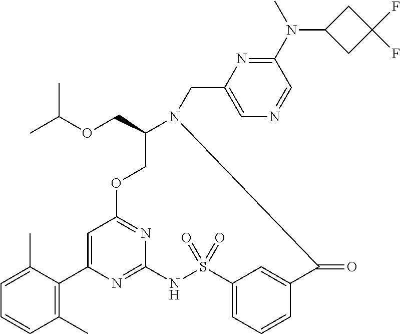 [SMILES: Cc1cccc(C)c1-c1cc2nc(n1)NS(=O)(=O)c1cccc(c1)C(=O)N(Cc1cncc(N(C)C3CC(F)(F)C3)n1)[C@H](COC(C)C)CO2] | | | | | | +++ |
| 1947 | 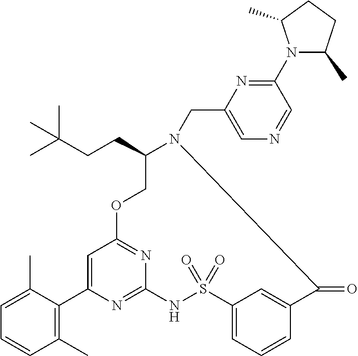 [SMILES: Cc1cccc(C)c1-c1cc2nc(n1)NS(=O)(=O)c1cccc(c1)C(=O)N(Cc1cncc(N3[C@H](C)CC[C@H]3C)n1)[C@H](CCC(C)(C)C)CO2] | | | | | | +++ |
| 1948 | 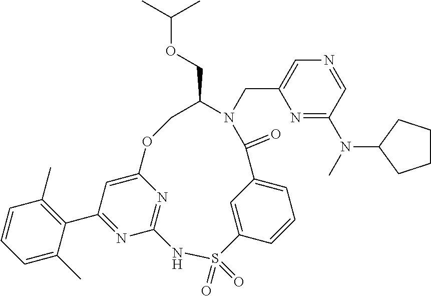 [SMILES: Cc1cccc(C)c1-c1cc2nc(n1)NS(=O)(=O)c1cccc(c1)C(=O)N(Cc1cncc(N(C)C3CCCC3)n1)[C@H](COC(C)C)CO2] | | | | | | +++ |
| 1949 | 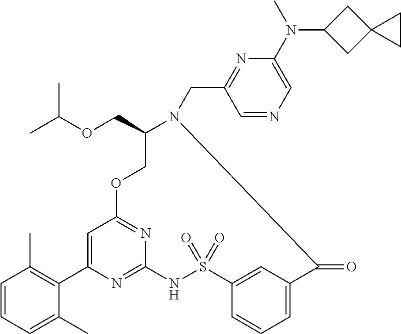 [SMILES: Cc1cccc(C)c1-c1cc2nc(n1)NS(=O)(=O)c1cccc(c1)C(=O)N(Cc1cncc(N(C)C3CC4(CC4)C3)n1)[C@H](COC(C)C)CO2] | | | | | | +++ |
| 1950 | 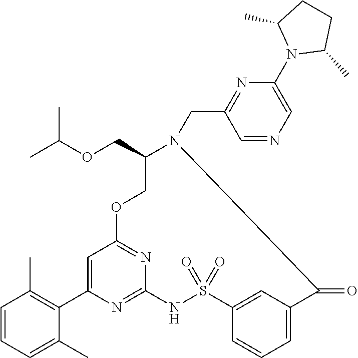 [SMILES: Cc1cccc(C)c1-c1cc2nc(n1)NS(=O)(=O)c1cccc(c1)C(=O)N(Cc1cncc(N3[C@H](C)CC[C@@H]3C)n1)[C@H](COC(C)C)CO2] | | | | | | +++ |
| 1951 | 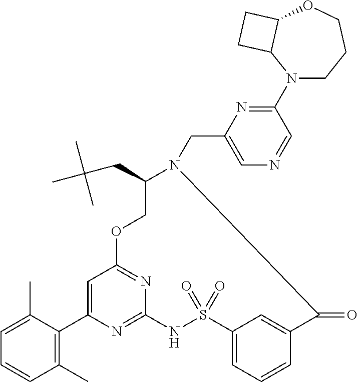 | | | | | | +++ |
| 1952 | 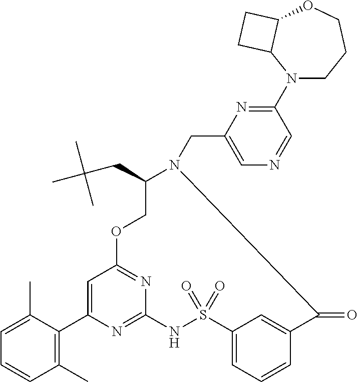 | | | | | | +++ |
| 1953 | 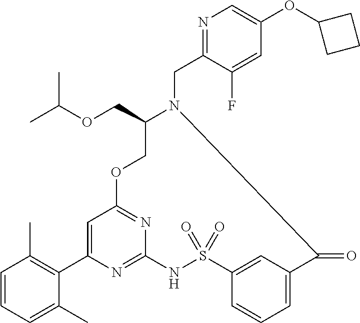 [SMILES: Cc1cccc(C)c1-c1cc2nc(n1)NS(=O)(=O)c1cccc(c1)C(=O)N(Cc1ncc(OC3CCC3)cc1F)[C@H](COC(C)C)CO2] | | | | | | +++ |
| 1954 | 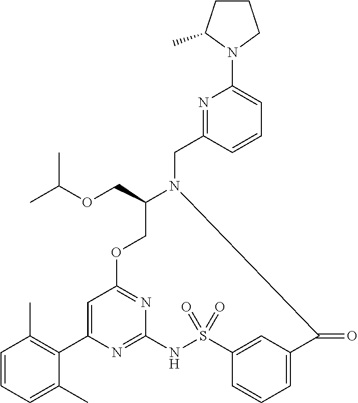 [SMILES: Cc1cccc(C)c1-c1cc2nc(n1)NS(=O)(=O)c1cccc(c1)C(=O)N(Cc1cccc(N3CCC[C@H]3C)n1)[C@H](COC(C)C)CO2] | | | | | | +++ |
| 1955 | 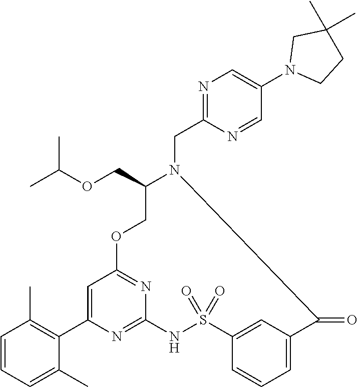 [SMILES: Cc1cccc(C)c1-c1cc2nc(n1)NS(=O)(=O)c1cccc(c1)C(=O)N(Cc1ncc(N3CCC(C)(C)C3)cn1)[C@H](COC(C)C)CO2] | | | | | | +++ |
| 1956 | 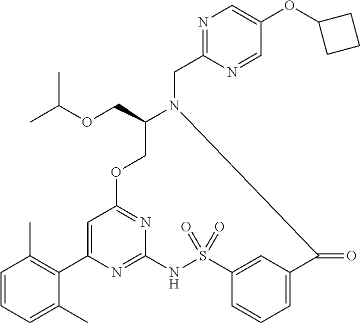 [SMILES: Cc1cccc(C)c1-c1cc2nc(n1)NS(=O)(=O)c1cccc(c1)C(=O)N(Cc1ncc(OC3CCC3)cn1)[C@H](COC(C)C)CO2] | | | | | | +++ |
| 1957 | 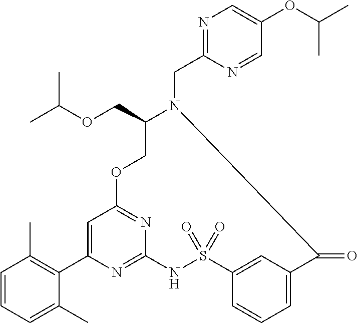 [SMILES: Cc1cccc(C)c1-c1cc2nc(n1)NS(=O)(=O)c1cccc(c1)C(=O)N(Cc1ncc(OC(C)C)cn1)[C@H](COC(C)C)CO2] | | | | | | +++ |
| 1958 | 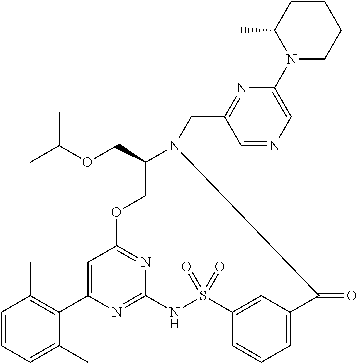 [SMILES: Cc1cccc(C)c1-c1cc2nc(n1)NS(=O)(=O)c1cccc(c1)C(=O)N(Cc1cncc(N3CCCC[C@H]3C)n1)[C@H](COC(C)C)CO2] | | | | | | |
| 1959 | 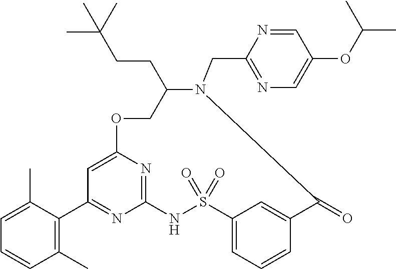 [SMILES: Cc1cccc(C)c1-c1cc2nc(n1)NS(=O)(=O)c1cccc(c1)C(=O)N(Cc1ncc(OC(C)C)cn1)C(CCC(C)(C)C)CO2] | | | | | | |
| 1960 | 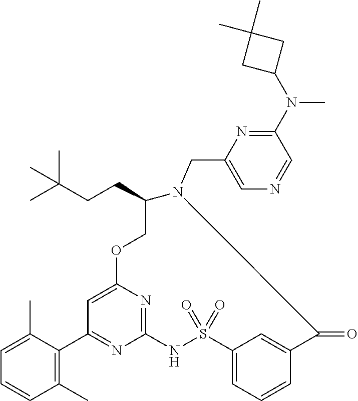 [SMILES: Cc1cccc(C)c1-c1cc2nc(n1)NS(=O)(=O)c1cccc(c1)C(=O)N(Cc1cncc(N(C)C3CC(C)(C)C3)n1)[C@H](CCC(C)(C)C)CO2] | | | | | | |
| 1961 | 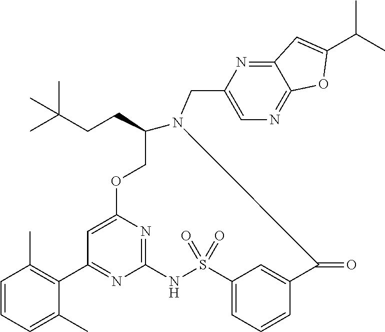 [SMILES: Cc1cccc(C)c1-c1cc2nc(n1)NS(=O)(=O)c1cccc(c1)C(=O)N(Cc1cnc3oc(C(C)C)cc3n1)[C@H](CCC(C)(C)C)CO2] | | | | | | |
| 1962 | 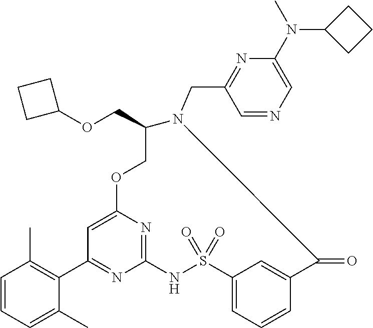 [SMILES: Cc1cccc(C)c1-c1cc2nc(n1)NS(=O)(=O)c1cccc(c1)C(=O)N(Cc1cncc(N(C)C3CCC3)n1)[C@H](COC1CCC1)CO2] | | | | | | |
| 1963 | 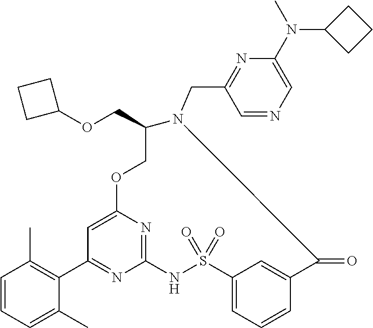 [SMILES: Cc1cccc(C)c1-c1cc2nc(n1)NS(=O)(=O)c1cccc(c1)C(=O)N(Cc1cncc(N(C)C3CCC3)n1)[C@H](COC1CCC1)CO2] | | | | | | |
| 1964 | 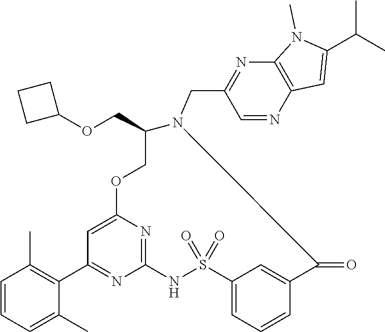 [SMILES: Cc1cccc(C)c1-c1cc2nc(n1)NS(=O)(=O)c1cccc(c1)C(=O)N(Cc1cnc3cc(C(C)C)n(C)c3n1)[C@H](COC1CCC1)CO2] | | | | | | |
| 1965 | 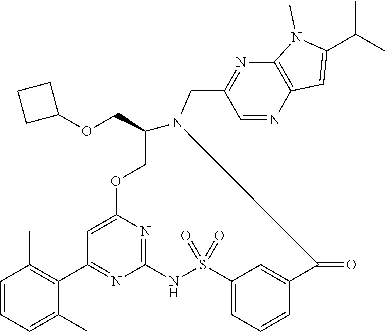 [SMILES: Cc1cccc(C)c1-c1cc2nc(n1)NS(=O)(=O)c1cccc(c1)C(=O)N(Cc1cnc3cc(C(C)C)n(C)c3n1)[C@H](COC1CCC1)CO2] | | | | | | |
| 1966 | 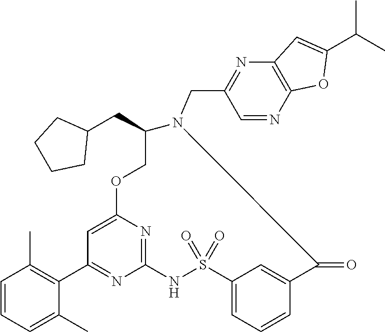 [SMILES: Cc1cccc(C)c1-c1cc2nc(n1)NS(=O)(=O)c1cccc(c1)C(=O)N(Cc1cnc3oc(C(C)C)cc3n1)[C@H](CC1CCCC1)CO2] | | | | | | |
| 1967 | 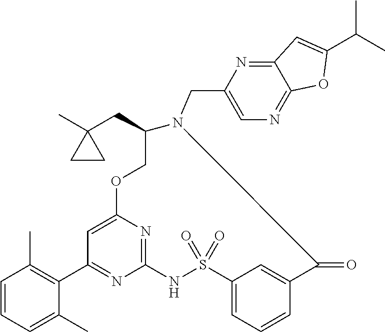 [SMILES: Cc1cccc(C)c1-c1cc2nc(n1)NS(=O)(=O)c1cccc(c1)C(=O)N(Cc1cnc3oc(C(C)C)cc3n1)[C@H](CC1(C)CC1)CO2] | | | | | | |
| 1968 | 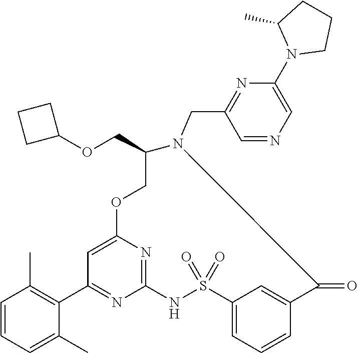 [SMILES: Cc1cccc(C)c1-c1cc2nc(n1)NS(=O)(=O)c1cccc(c1)C(=O)N(Cc1cncc(N3CCC[C@H]3C)n1)[C@H](COC1CCC1)CO2] | | | | | | |
| 1969 | 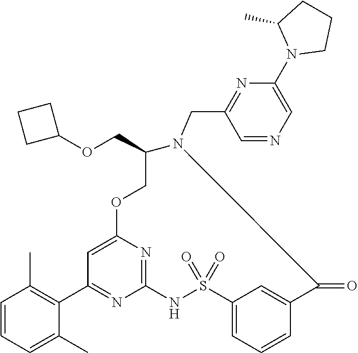 [SMILES: Cc1cccc(C)c1-c1cc2nc(n1)NS(=O)(=O)c1cccc(c1)C(=O)N(Cc1cncc(N3CCC[C@H]3C)n1)[C@H](COC1CCC1)CO2] | | | | | | |
| 1970 | 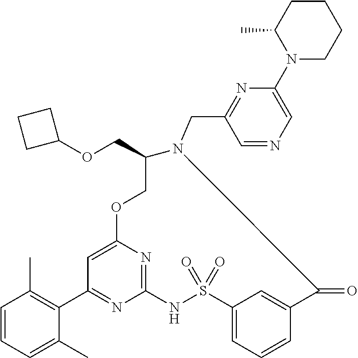 [SMILES: Cc1cccc(C)c1-c1cc2nc(n1)NS(=O)(=O)c1cccc(c1)C(=O)N(Cc1cncc(N3CCCC[C@H]3C)n1)[C@H](COC1CCC1)CO2] | | | | | | |
| 1971 | 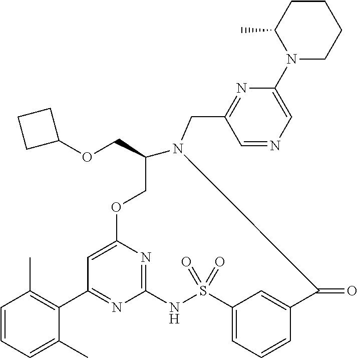 [SMILES: Cc1cccc(C)c1-c1cc2nc(n1)NS(=O)(=O)c1cccc(c1)C(=O)N(Cc1cncc(N3CCCC[C@H]3C)n1)[C@H](COC1CCC1)CO2] | | | | | | |
| 1972 | 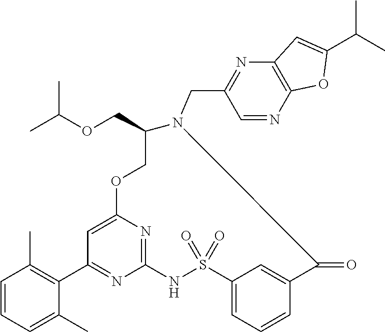 [SMILES: Cc1cccc(C)c1-c1cc2nc(n1)NS(=O)(=O)c1cccc(c1)C(=O)N(Cc1cnc3oc(C(C)C)cc3n1)[C@H](COC(C)C)CO2] | | | | | | |

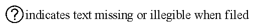

### VIL. Synthesis of (6R,12R)-17-amino-12-methyl-6,15-bis(trifluoromethyl)-13,19-dioxa-3,4,18-triazatricyclo [12.3.1.12,51 nonadeca-1(18),2,4,14,16-pentaen-6-ol

### A. General Methods

Reagents and starting materials were obtained by commercial sources unless otherwise stated and were used without purification.

Proton and carbon NMR spectra were acquired on either a Bruker Biospin DRX 400 MHz FTNMR spectrometer operating at a ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters.

NMR (1D & 2D) spectra were also recorded on a Bruker AVNEO 400 MHz spectrometer operating at 400 MHz and 100 MHz respectively equipped with a 5 mm multinuclear Iprobe.

NMR spectra were also recorded on a Varian Mercury NMR instrument at 300 MHz for ¹H using a 45 degree pulse angle, a spectral width of 4800 Hz and 28860 points of acquisition. FID were zero-filled to 32 k points and a line broadening of 0.3 Hz was applied before Fourier transform. ¹⁹F NMR spectra were recorded at 282 MHz using a 30 degree pulse angle, a spectral width of 100 kHz and 59202 points were acquired. FID were zero-filled to 64 k points and a line broadening of 0.5 Hz was applied before Fourier transform.

NMR spectra were also recorded on a Bruker Avance III HD NMR instrument at 400 MHz for ¹H using a 30 degree pulse angle, a spectral width of 8000 Hz and 128 k points of acquisition. FID were zero-filled to 256 k points and a line broadening of 0.3 Hz was applied before Fourier transform. ¹⁹F NMR spectra were recorded at 377 MHz using a 30 deg pulse angle, a spectral width of 89286 Hz and 128 k points were acquired. FID were zero-filled to 256 k points and a line broadening of 0.3 Hz was applied before Fourier transform.

NMR spectra were also recorded on a Bruker AC 250 MHz instrument equipped with a: 5 mm QNP(H1/C13/F19/P31) probe (type: 250-SB, s #23055/0020) or on a Varian 500 MHz instrument equipped with a ID PFG, 5 mm, 50-202/500 MHz probe (model/part #99337300).

Unless stated to the contrary in the following examples, final purity of compounds was determined by reversed phase UPLC using an Acquity UPLC BEH C₁₈ column (50×2.1 mm, 1.7 m particle) made by Waters (pn: 186002350), and a dual gradient run from 1-99% mobile phase B over 3.0 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C. Final purity was calculated by averaging the area under the curve (AUC) of two UV traces (220 nm, 254 nm). Low-resolution mass spectra were reported as [M+1]⁺ species obtained using a single quadrupole mass spectrometer equipped with an electrospray ionization (ESI) source capable of achieving a mass accuracy of 0.1 Da and a minimum resolution of 1000 (no units on resolution) across the detection range.

Solid-state NMR (SSNMR) spectra were recorded on a Bruker-Biospin 400 MHz wide-bore spectrometer equipped with Bruker-Biospin 4 mm HFX probe. Samples were packed into 4 mm ZrO2 rotors and spun under Magic Angle Spinning (MAS) condition with spinning speed typically set to 12.5 kHz. The proton relaxation time was measured using ¹H MAS Ti saturation recovery relaxation experiment in order to set up proper recycle delay of the ¹³C cross-polarization (CP) MAS experiment. The fluorine relaxation time was measured using ¹⁹F MAS Ti saturation recovery relaxation experiment in order to set up proper recycle delay of the ¹⁹F MAS experiment. The CP contact time of carbon CPMAS experiment was set to 2 ms. A CP proton pulse with linear ramp (from 50% to 100%) was employed. The carbon Hartmann-Hahn match was optimized on external reference sample (glycine). Both carbon and fluorine spectra were recorded with proton decoupling using TPPM15 decoupling sequence with the field strength of approximately 100 kHz.

### B. Procedures for the Synthesis of Intermediates

### Intermediate 1: Preparation of methyl 3-[bis(tert-butoxycarbonyl)amino]-6-bromo-5-(trifluoromethyl)pyridine-2-carboxylate

### Step 1: Methyl 3-(benzhydrylideneamino)-5-(trifluoromethyl)pyridine-2-carboxylate

A mixture of methyl 3-chloro-5-(trifluoromethyl)pyridine-2-carboxylate (47.3 g, 197.43 mmol), diphenylmethanimine (47 g, 259.33 mmol), Xantphos (9.07 g, 15.675 mmol), and cesium carbonate (131 g, 402.06 mmol) in dioxane (800 mL) was degassed with bubbling nitrogen for 30 minutes. Pd(OAc)₂ (3.52 g, 15.679 mmol) was added and the system was purged with nitrogen three times. The reaction mixture was heated at 100° C. for 18 h. The reaction was cooled to room temperature and filtered on a pad of Celite. The cake was washed with EtOAc and solvents were evaporated under reduced pressure to give methyl 3-(benzhydrylideneamino)-5-(trifluoromethyl)pyridine-2-carboxylate (90 g, 84%) as yellow solid. ESI-MS m z calc. 384.10855, found 385.1 (M+1)⁺; Retention time: 2.24 minutes. LCMS Method: Kinetex C₁₈ 4.6×50 mm 2.6 μM, 2.0 mL/min, 95% H₂O (0.1% formic acid)+5% acetonitrile (0.1% formic acid) to 95% acetonitrile (0.1% formic acid) gradient (2.0 min) then held at 95% acetonitrile (0.1% formic acid) for 1.0 min.

### Step 2: Methyl 3-amino-5-(trifluoromethyl)pyridine-2-carboxylate

To a suspension of methyl 3-(benzhydrylideneamino)-5-(trifluoromethyl)pyridine-2-carboxylate (65 g, 124.30 mmol) in methanol (200 mL) was added HCl (3 M in methanol) (146 mL of 3 M, 438.00 mmol). The mixture was stirred at room temperature for 1.5 hour then the solvent was removed under reduced pressure. The residue was taken up in ethyl acetate (2 L) and dichloromethane (500 mL). The organic phase was washed with 5% aqueous sodium bicarbonate solution (3×500 mL) and brine (2×500 mL), dried over anhydrous sodium sulfate, filtered and the solvent was removed under reduced pressure. The residue was triturated with heptanes (2×50 mL) and the mother liquors were discarded. The solid obtained was triturated with a mixture of dichloromethane and heptanes (1:1, 40 mL) and filtered to afford methyl 3-amino-5-(trifluoromethyl)pyridine-2-carboxylate (25.25 g, 91%) as yellow solid. ¹H NMR (300 MHz, CDCl₃) δ 8.24 (s, 1H), 7.28 (s, 1H), 5.98 (br. s, 2H), 4.00 (s, 3H) ppm. 19F NMR (282 MHz, CDCl₃) δ-63.23 (s, 3F) ppm. ESI-MS m/z calc. 220.046, found 221.1 (M+1)⁺; Retention time: 1.62 minutes. LCMS Method: Kinetex Polar C₁₈ 3.0×50 mm 2.6 μm, 3 min, 5-95% acetonitrile in H₂O (0.1% formic acid) 1.2 mL/min.

### Step 3: Methyl 3-amino-6-bromo-5-(trifluoromethyl)pyridine-2-carboxylate

To a solution of methyl 3-amino-5-(trifluoromethyl)pyridine-2-carboxylate (18.75 g, 80.91 mmol) in acetonitrile (300 mL) at 0° C. was added portion wise N-bromosuccinimide (18.7 g, 105.3 mmol). The mixture was stirred overnight at 25° C. Ethyl acetate (1000 mL) was added. The organic layer was washed with 10% sodium thiosulfate solution (3×200 mL) which were back extracted with ethyl acetate (2×200 mL). The combined organic extracts were washed with saturated sodium bicarbonate solution (3×200 mL), brine (200 mL), dried over sodium sulfate and concentrated in vacuo to provide methyl 3-amino-6-bromo-5-(trifluoromethyl)pyridine-2-carboxylate (25.46 g, 98%). ¹H NMR (300 MHz, CDCl₃) δ 3.93-4.03 (m, 3H), 6.01 (br. s., 2H), 7.37 (s, 1H) ppm. ¹⁹F NMR (282 MHz, CDCl₃) ppm −64.2 (s, 3F). ESI-MS m/z calc. 297.9565, found 299.0 (M+1)⁺; Retention time: 2.55 minutes. LCMS Method: Kinetex C₁₈ 4.6×50 mm 2.6 μM. Temp: 45° C., Flow: 2.0 mL/min, Run Time: 6 min. Mobile Phase: Initial 95% H₂O (0.1% formic acid) and 5% acetonitrile (0.1% formic acid) linear gradient to 95% acetonitrile (0.1% formic acid) for 4.0 min then held at 95% acetonitrile (0.1% formic acid) for 2.0 min.

### Step 4: Methyl 3-[bis(tert-butoxycarbonyl)amino]-6-bromo-5-(trifluoro methyl)pyridine-2-carboxylate

A mixture of methyl 3-amino-6-bromo-5-(trifluoromethyl)pyridine-2-carboxylate (5 g, 15.549 mmol), (Boc)₂₀ (11 g, 11.579 mL, 50.402 mmol), DMAP (310 mg, 2.5375 mmol) and CH₂C₁₂ (150 mL) was stirred at room temperature overnight. The reaction mixture was concentrated under reduced pressure and purification by silica gel chromatography (0-15% ethyl acetate in heptane) provided methyl 3-[bis(tert-butoxycarbonyl)amino]-6-bromo-5-(trifluoromethyl)pyridine-2-carboxylate (6.73 g, 87%) as light yellow solid. ¹H NMR (300 MHz, CDCl₃) δ 1.42 (s, 18H), 3.96 (s, 3H), 7.85 (s, 1H) ppm. ¹⁹F NMR (282 MHz, CDCl₃) δ-63.9 (s, 3F) ppm. ESI-MS m/z calc. 498.06134, Retention time: 2.34 minutes. LCMS Method: Kinetex C₁₈ 4.6×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Intermediate 2: Preparation of 6-bromo-3-(tert-butoxycarbonylamino)-5-(trifluoromethyl)pyridine-2-carboxylic acid

### Step 1: 6-Bromo-3-(tert-butoxycarbonylamino)-5-(trifluoromethyl)pyridine-2-carboxylic acid

To a mixture of methyl 3-[bis(tert-butoxycarbonyl)amino]-6-bromo-5-(trifluoromethyl)pyridine-2-carboxylate (247 g, 494.7 mmol) in THE (1.0 L) was added a solution of LiOH (47.2 g, 1.971 mol) in water (500 mL). The mixture was stirred at ambient temperature for 18 h affording a yellow slurry. The mixture was cooled with an ice-bath and slowly acidified with HCl (1000 mL of 2 M, 2.000 mol) keeping the reaction temperature<15° C. The mixture was diluted with heptane (1.5 L), mixed and the organic phase separated. The aqueous phase was extracted with heptane (500 mL). The combined organic phases were washed with brine, dried over MgSO₄, filtered and concentrated in vacuo. The crude oil was dissolved in heptane (600 mL), seeded and stirred at ambient temperature for 18 h affording a thick slurry. The slurry was diluted with cold heptane (500 mL) and the precipitate collected using a medium frit. The filter cake was washed with cold heptane and air dried for 1 h, then in vacuo at 45° C. for 48 h to afford 6-bromo-3-(tert-butoxycarbonylamino)-5-(trifluoromethyl)pyridine-2-carboxylic acid (158.3 g, 83%). ¹H NMR (400 MHz, DMSO-d6) δ 10.38 (s, 1H), 9.01 (s, 1H), 1.50 (s, 9H) ppm. ESI-MS m z calc. 383.99326, found 384.9 (M+1)⁺; Retention time: 2.55 minutes. LCMS Method Detail: Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=acetonitrile (0.035% CF₃CO₂H). Flow rate=1.2 mL/min, injection volume=1.5 μL, and column temperature=60° C.

### Intermediate 3: Preparation of 2-Benzyloxy-2-(trifluoromethyl)hex-5-enoic acid

### Step 1: Ethyl 2-hydroxy-2-(trifluoromethyl)hex-5-enoate

To a solution of ethyl 3,3,3-trifluoro-2-oxo-propanoate (25.15 g, 147.87 mmol) in Et₂O (270 mL) at −78° C. was added bromo(but-3-enyl)magnesium in THE (190 mL of 0.817 M, 155.23 mmol) dropwise over a period of 1.5 h (inner temperature −72° C. to −76° C.). The mixture was stirred at −78° C. for 20 min. The dry ice-acetone bath was removed. The mixture was slowly warm to 5° C. during 1 h, added to a mixture of 1 N aqueous HCl (170 mL) and crushed ice (150 g) (pH=4). The two layers were separated. The organic layer was concentrated, and the residue was combined with aqueous phase and extracted with EtOAc (2×150 mL). The combined organic phase was washed with 5% aqueous NaHCO₃(50 mL) and brine (20 mL), dried with Na₂SO₄. The mixture was filtered and concentrated, and co-evaporated with THE (2×40 mL) to give ethyl 2-hydroxy-2-(trifluoromethyl)hex-5-enoate (37.44 g, 96%) as colorless oil. ¹H NMR (300 MHz, CDCl₃) δ 5.77 (ddt, J=17.0, 10.4, 6.4 Hz, 1H), 5.15-4.93 (m, 2H), 4.49-4.28 (m, 2H), 3.88 (s, 1H), 2.35-2.19 (m, 1H), 2.17-1.89 (m, 3H), 1.34 (t, J=7.0 Hz, 3H) ppm. ¹⁹F NMR (282 MHz, CDCl₃) δ −78.74 (s, 3F) ppm.

### Step 2: Ethyl 2-benzyloxy-2-(trifluoromethyl)hex-5-enoate

To a solution of ethyl 2-hydroxy-2-(trifluoromethyl)hex-5-enoate (24.29 g, 87.6% purity, 94.070 mmol) in DMF (120 mL) at 0° C. was added NaH (60% in mineral oil, 5.64 g, 141.01 mmol) portion-wise. The mixture was stirred at 0° C. for 10 min. Benzyl bromide (24.13 g, 141.08 mmol) and TBAI (8.68 g, 23.500 mmol) were added. The mixture was stirred at room temperature overnight. NH₄Cl (3 g, 0.6 eq) was added. The mixture was stirred for 10 min. 30 mL of EtOAc was added, then ice-water was added (400 g). The mixture was extracted with CH₂C₁₂ and the combined organic layers were concentrated. Purification by silica gel chromatography (0-20% CH₂C₁₂ in heptanes) provided ethyl 2-benzyloxy-2-(trifluoromethyl)hex-5-enoate (26.05 g, 88%) as pink oil. ¹H NMR (300 MHz, CDCl₃) δ 1.34 (t, J=7.2 Hz, 3H), 2.00-2.19 (m, 3H), 2.22-2.38 (m, 1H), 4.33 (q, J=7.2 Hz, 2H), 4.64 (d, J=10.6 Hz, 1H), 4.84 (d, J=10.9 Hz, 1H), 4.91-5.11 (m, 2H), 5.62-5.90 (m, 1H), 7.36 (s, 5H) ppm. 19F NMR (282 MHz, CDCl₃) δ −70.5 (s, 3F) ppm. ESI-MS m/z calc. 316.12863, found 317.1 (M+1)⁺; Retention time: 2.47 minutes. LCMS Method: Kinetex C₁₈ 4.6×50 mm 2.6 μM. Temp: 45° C., Flow: 2.0 mL/min, Run Time: 3 min. Mobile Phase: Initial 95% H₂O (0.1% formic acid) and 5% acetonitrile (0.1% formic acid) linear gradient to 95% acetonitrile (0.1% formic acid) for 2.0 min then held at 95% acetonitrile (0.1% formic acid) for 1.0 min.

### Step 3: 2-Benzyloxy-2-(trifluoromethyl)hex-5-enoic acid

A solution of sodium hydroxide (7.86 g, 196.51 mmol) in water (60 mL) was added to a solution of ethyl 2-benzyloxy-2-(trifluoromethyl)hex-5-enoate (24.86 g, 78.593 mmol) in methanol (210 mL). The reaction was heated at 50° C. overnight. The reaction was concentrated to remove methanol, diluted with water (150 mL) and the carboxylate sodium salt was washed with heptane (1×100 mL). The aqueous solution was acidified to pH=2 with aqueous 3N solution of HCl. The carboxylic acid was extracted with dichloromethane (3×100 mL) and dried over sodium sulfate. The solution was filtered and concentrated to give 2-benzyloxy-2-(trifluoromethyl)hex-5-enoic acid (22.57 g, 97%) as pale yellow oil. ¹H NMR (300 MHz, DMSO-d6) δ 14.31 (br. s., 1H), 7.55-7.20 (m, 5H), 5.93-5.70 (m, 1H), 5.17-4.91 (m, 2H), 4.85-4.68 (m, 1H), 4.67-4.55 (m, 1H), 2.32-1.94 (m, 4H) ppm. ¹⁹F NMR (282 MHz, DMSO-d6) δ-70.29 (s, 3F) ppm. ESI-MS m/z calc. 288.09732, found 287.1 (M-1); Retention time: 3.1 minutes. LCMS Method: Kinetex Polar C₁₈ 3.0×50 mm 2.6 μm, 6 min, 5-95% acetonitrile in H₂O (0.1% formic acid) 1.2 mL/min.

### Intermediate 4: Preparation of (2R)-2-benzyloxy-2-(trifluoromethyl)hex-5-enoic acid

### Step-1: (2R)-2-Benzyloxy-2-(trifluoromethyl)hex-5-enoic acid; (R)-4-quinolyl-[(2S,4S)-5-vinylquinuclidin-2-yl]methanol

To a N₂ purged jacketed reactor set to 20° C. was added isopropyl acetate (IPAC, 100 L, 0.173 M, 20 Vols), followed by previously melted 2-benzyloxy-2-(trifluoromethyl)hex-5-enoic acid (5.00 kg, 17.345 mol) and cinchonidine (2.553 kg, 8.67 mol) made into a slurry with minor amount of the reaction solvent. The reactor was set to ramp internal temperature to 80° C. over 1 hour, with solids going in solution upon heating to set temperature, then the solution was held at temperature for at least 10 minutes, then cooled to 70° C. held and seeded with chiral salt (50 g, 1.0% by wt). The mixture was stirred for 10 minutes, then ramped to 20° C. internal temperature over 4 hours, then held overnight at 20° C. The mixture was filtered, cake washed with isopropyl acetate (10.0 L, 2.0 vols) and dried under vacuum. The cake was then dried in vacuo (50° C., vacuum) to afford 4.7 kg of salt. The resulting solid salt was returned to the reactor by making a slurry with a portion of isopropyl acetate (94 L, 20 vol based on current salt wt), and pumped into reactor and stirred. The mixture was then heated to 80° C. internal, stirred hot slurry for at least 10 minutes, then ramped to 20° C. over 4-6 h, then stirred overnight at 20° C. The material was then filtered and cake washed with isopropyl acetate (9.4 L, 2.0 vol), pulled dry, cake scooped out and dried in vacuo (50° C., vacuum) to afford 3.1 kg of solid. The solid (3.1 kg) and isopropyl acetate (62 L, 20 vol based on salt solid wt) was slurried and added to a reactor, stirred under N₂ purge and heated to 80° C. and held at temperature at least 10 minutes, then ramped to 20° C. over 4-6 hours, then stirred overnight. The mixture was filtered, cake washed with isopropyl acetate (6.2 L, 2 vol), pulled dry, scooped out and dried in vacuo (50° C., vac) to afford 2.25 kg of solid salt. The solid (2.25 kg) and isopropyl acetate (45 L, 20 vol based on salt solid wt) was slurried and added to a reactor, stirred under N₂ purge and heated to 80° C., held at temperature at least 10 minutes, then ramped to 20° C. over 4-6 hours, then stirred overnight. The mixture was filtered, cake washed with isopropyl acetate (4.5 L, 2 vol), pulled dry, scooped out and dried in vacuo (50° C. to afford (2R)-2-benzyloxy-2-(trifluoromethyl)hex-5-enoic acid;(R)-4-quinolyl-[(2S,4S)-5-vinylquinuclidin-2-yl]methanol (1.886 kg, >98.0% ee) as off-white to tan solid. Chiral purity was determined by Agilent 1200 HPLC instrument using Phenomenex Lux i-Amylose-3 column (3 μm, 150×4.6 mm) and a dual, isocratic gradient run 30% to 70% mobile phase B over 20.0 minutes. Mobile phase A=H₂O (0.1% CF₃CO₂H). Mobile phase B=MeOH (0.1% CF₃CO₂H). Flow rate=1.0 mL/min, injection volume=2 μL, and column temperature=30° C., sample concentration: 1 mg/mL in 60% acetonitrile/40% water.

### Step 2: (2R)-2-Benzyloxy-2-(trifluoromethyl)hex-5-enoic acid

A suspension of (2R)-2-benzyloxy-2-(trifluoromethyl)hex-5-enoic acid; (R)-4-quinolyl-[(2S,4S)-5-vinylquinuclidin-2-yl]methanol (50 g, 87.931 mmol) in ethyl acetate (500.00 mL) was treated with an aqueous solution of hydrochloric acid (200 mL of 1 M, 200.00 mmol). After stirring 15 minutes at room temperature, the two phases were separated. The aqueous phase was extracted twice with ethyl acetate (200 mL). The combined organic layer was washed with 1 N HCl (100 mL). The organic layer was dried over sodium sulfate, filtered and concentrated. The material was dried over high vacuum overnight to give (2R)-2-benzyloxy-2-(trifluoromethyl)hex-5-enoic acid (26.18 g, 96%) as pale brown oil. ¹H NMR (400 MHz, CDCl₃) δ 7.46-7.31 (m, 5H), 5.88-5.73 (m, 1H), 5.15-4.99 (m, 2H), 4.88 (d, J=10.3 Hz, 1H), 4.70 (d, J=10.3 Hz, 1H), 2.37-2.12 (m, 4H) ppm. ¹⁹F NMR (377 MHz, CDCl₃) δ-71.63 (br s, 3F) ppm. ESI-MS m/z calc. 288.0973, found 287.0 (M-1); Retention time: 2.15 minutes. LCMS Method: Kinetex Polar C₁₈ 3.0×50 mm 2.6 μm, 3 min, 5-95% acetonitrile in H₂O (0.1% formic acid) 1.2 mL/min.

### Intermediate 5: Preparation of (2R)-2-benzyloxy-2-(trifluoromethyl)hex-5-enehydrazide

### Step 1: tert-Butyl N-[[(2R)-2-benzyloxy-2-(trifluoromethyl)hex-5-enoyl]amino]carbamate

To a solution of (2R)-2-benzyloxy-2-(trifluoromethyl)hex-5-enoic acid (365 g, 1.266 mol) in DMF (2 L) was added HATU (612 g, 1.610 mol) and DIEA (450 mL, 2.584 mol) and the mixture was stirred at ambient temperature for 10 min. To the mixture was added tert-butyl N-aminocarbamate (200 g, 1.513 mol) (slight exotherm upon addition) and the mixture was stirred at ambient temperature for 16 h. The reaction was poured into ice water (5 L). The resultant precipitate was collected by filtration and washed with water. The solid was dissolved in EtOAc (2 L) and washed with brine. The organic phase was dried over MgSO₄, filtered and concentrated in vacuo. The oil was diluted with EtOAc (500 mL) followed by heptane (3 L) and stirred at ambient temperature for several hours affording a thick slurry. The slurry was diluted with additional heptane and filtered to collect fluffy white solid (343 g). The filtrate was concentrated and purification by silica gel chromatography (0-40% EtOAc/hexanes) provided tert-butyl N-[[(2R)-2-benzyloxy-2-(trifluoromethyl)hex-5-enoyl]amino]carbamate (464 g, 91%, combined with product from crystallization). ESI-MS m z calc. 402.17664, found 303.0 (M+1-Boc)⁺; Retention time: 2.68 minutes. Final purity was determined by reversed phase UPLC using an Acquity UPLC BEH C₁₈ column (50×2.1 mm, 1.7 m particle) made by Waters (pn: 186002350) and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 2: (2R)-2-Benzyloxy-2-(trifluoromethyl)hex-5-enehydrazide

To a solution of tert-butyl N-[[(2R)-2-benzyloxy-2-(trifluoromethyl)hex-5-enoyl]amino]carbamate (464 g, 1.153 mol) in DCM (1.25 L) and was added HCl (925 mL of 4 M, 3.700 mol) and the mixture stirred at ambient temperature for 20 h. The mixture was concentrated in vacuo removing most of the DCM. The mixture was diluted with isopropyl acetate (1 L) and basified to pH=6 with NaOH (140 g of 50% w/w, 1.750 mol) in 1 L of ice water. The organic phase was separated and washed with IL of brine and the combined aqueous phases were extracted with isopropyl acetate (1 L). The combined organic phases were dried over MgSO₄, filtered and concentrated in vacuo affording a dark yellow oil of (2R)-2-benzyloxy-2-(trifluoromethyl)hex-5-enehydrazide (358 g, quant.). ¹H NMR (400 MHz, CDCl₃) δ 8.02 (s, 1H), 7.44-7.29 (m, 5H), 5.81 (ddt, J=16.8, 10.1, 6.4 Hz, 1H), 5.13-4.93 (m, 2H), 4.75 (dd, J=10.5, 1.5 Hz, 1H), 4.61 (d, J=10.5 Hz, 1H), 3.78 (s, 2H), 2.43 (ddd, J=14.3, 11.0, 5.9 Hz, 1H), 2.26-1.95 (m, 3H) ppm. ESI-MS m z calc. 302.1242, found 303.0 (M+1)⁺; Retention time: 2.0 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 6: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

### Step 1: tert-Butyl N-[2-[[[(2R)-2-benzyloxy-2-(trifluoromethyl)hex-5-enoyl]amino]carbamoyl]-6-bromo-5-(trifluoromethyl)-3-pyridyl]carbamate

To a mixture of 6-bromo-3-(tert-butoxycarbonylamino)-5-(trifluoromethyl)pyridine-2-carboxylic acid (304 g, 789.3 mmol) and (2R)-2-benzyloxy-2-(trifluoromethyl)hex-5-enehydrazide (270 g, 893.2 mmol) in EtOAc (2.25 L) at ambient temperature was added DIEA (425 mL, 2.440 mol). To the mixture was slowly added T3P (622 g of 50% w/w, 977.4 mmol) using an ice-water bath to keep the temperature<35° C. (temperature rose to 34° C.) and the reaction mixture was stirred at ambient temperature for 18 h. Added additional DIEA (100 mL, 574.1 mmol) and T3P (95 g, 298.6 mmol) and stirred at ambient temperature for 2 days. Starting material was still observed and an additional T3P (252 g, 792 mmol) was added and stirred for 5 days. The reaction was quenched with the slow addition of water (2.5 L) and the mixture stirred for 30 min. The organic phase was separated, and the aqueous phase extracted with EtOAc (2 L). The combined organic phases were washed with brine, dried over MgSO₄, filtered and concentrated in vacuo. The crude product was dissolved in MTBE (300 mL) and diluted with heptane (3 L), the mixture stirred at ambient temperature for 12 h affording a light yellow slurry. The slurry was filtered, and the resultant solid was air dried for 2 h, then in vacuo at 40° C. for 48 h. The filtrate was concentrated in vacuo and purified by silica gel chromatography (0-20% EtOAc/hexanes) and combined with material obtained from crystallization providing tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (433 g, 82%). ¹H NMR (400 MHz, DMSO) δ 11.07 (s, 1H), 10.91 (s, 1H), 10.32 (s, 1H), 9.15 (s, 1H), 7.53-7.45 (m, 2H), 7.45-7.28 (m, 3H), 5.87 (ddt, J=17.0, 10.2, 5.1 Hz, 1H), 5.09 (dq, J=17.1, 1.3 Hz, 1H), 5.02 (dd, J=10.3, 1.9 Hz, 1H), 4.84 (q, J=11.3 Hz, 2H), 2.37-2.13 (m, 4H), 1.49 (s, 9H) ppm. ESI-MS m z calc. 668.1069, found 669.0 (M+1)⁺; Retention time: 3.55 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Step 2: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate

To a solution of tert-butyl N-[2-[[[(2R)-2-benzyloxy-2-(trifluoromethyl)hex-5-enoyl]amino]carbamoyl]-6-bromo-5-(trifluoromethyl)-3-pyridyl]carbamate (240 g, 358.5 mmol) in anhydrous acetonitrile (1.5 L) under nitrogen was added DIEA (230 mL, 1.320 mol) and the orange solution heated to 70° C. To the mixture was added p-toluenesulfonyl chloride (80.5 g, 422.2 mmol) in 3 equal portions over 1 h. The mixture was stirred at 70° C. for 9 h then additional p-toluenesulfonyl chloride (6.5 g, 34.09 mmol) was added. The mixture was stirred for a total of 24 h then allowed to cool to ambient temperature. Acetonitrile was removed in vacuo affording a dark orange oil which was diluted with EtOAc (1.5 L) and water (1.5 L). The organic phase was separated and washed with 500 mL of 1M HCl, 500 mL of brine, dried over MgSO₄, filtered and concentrated in vacuo. Purification by silica gel chromatography (0-20% EtOAc/hexanes) provided tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (200 g, 86%). ¹H NMR (400 MHz, DMSO) δ 10.11 (s, 1H), 9.10 (s, 1H), 7.55-7.48 (m, 2H), 7.47-7.28 (m, 3H), 5.87 (ddt, J=16.7, 10.2, 6.4 Hz, 1H), 5.11 (dt, J=17.2, 1.7 Hz, 1H), 5.01 (dt, J=10.2, 1.5 Hz, 1H), 4.74 (d, J=10.6 Hz, 1H), 4.65 (d, J=10.6 Hz, 1H), 2.55-2.42 (m, 2H), 2.30 (qd, J=11.3, 10.3, 6.9 Hz, 2H), 1.52 (s, 9H) ppm. ESI-MS m z calc. 650.0963, found 650.0 (M+1)⁺; Retention time: 3.78 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 7: Preparation of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate

To a solution of tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]carbamate (222 g, 340.8 mmol) in MTBE (1.333 L) was added DIPEA (65.3 mL, 374.9 mmol) followed DMAP (2.09 g, 17.11 mmol). Added a solution of di-tert-butyl dicarbonate (111.6 g, 511.3 mmol) in MTBE (250 mL) over approx. 8 minutes, and the resulting mixture was stirred for additional 30 min. Added 1 L of water and separated the layers. The organic layer was washed with KHSO₄ (886 mL of 0.5 M, 443.0 mmol), 300 mL brine, dried with MgSO₄ and most (>95%) of the MTBE was evaporated by rotary evaporation at 45° C., leaving a thick oil. Added 1.125 L of heptane, spun in the 45° C. rotovap bath until dissolved, then evaporated out 325 mL of solvent by rotary evaporation. The rotovap bath temp was allowed to drop to room temperature and product started crystallizing out during the evaporation. Then put the flask in a −20° C. freezer overnight. The resultant solid was filtered and washed with cold heptane and dried at room temperature for 3 days to give tert-butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (240.8 g, 94%). ¹H NMR (400 MHz, Chloroform-d) δ 7.95 (s, 1H), 7.52-7.45 (m, 2H), 7.44-7.36 (m, 2H), 7.36-7.29 (m, 1H), 5.83-5.67 (m, 1H), 5.08-5.00 (m, 1H), 5.00-4.94 (m, 1H), 4.79 (d, J=10.4 Hz, 1H), 4.64 (d, J=10.4 Hz, 1H), 2.57-2.26 (m, 3H), 2.26-2.12 (m, 1H), 1.41 (s, 18H) ppm. ESI-MS m z calc. 750.14874, found 751.1 (M+1)⁺; Retention time: 3.76 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### Intermediate 8: Preparation of tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

### Step 1: tert-Butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate

tert-Butyl N-[2-[5-[(1R)-1-benzyloxy-1-(trifluoromethyl)pent-4-enyl]-1,3,4-oxadiazol-2-yl]-6-bromo-5-(trifluoromethyl)-3-pyridyl]-N-tert-butoxycarbonyl-carbamate (280 g, 372.6 mmol) was dissolved in DMSO (1.82 L) (yellow solution) and treated with cesium acetate (215 g, 1.120 mol) under stirring at room temperature. The yellow suspension was heated at 80° C. for 5 h. The reaction mixture was cooled to room temperature and added to a stirred cold emulsion of water (5.5 L) with 1 kg ammonium chloride dissolved in it and a 1:1 mixture of MTBE and heptane (2 L) (in 20 L). The phases were separated and the organic phase washed water (3×3 L) and with brine (1×2.5 L). The organic phase was dried with MgSO₄, filtered and concentrated under reduced pressure. The resultant yellow solution was diluted with heptane (˜1 L) and seeded with tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate and stirred on the rotovap at 100 mbar pressure at room temperature for 1.5 h. The solid mass was stirred mechanically for 2 h at room temperature, resultant thick fine suspension was filtered, washed with dry ice cold heptane and dried under vacuum at 45° C. with a nitrogen bleed for 16 h to give tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (220 g, 85%) as an off white solid. ¹H NMR (400 MHz, DMSO-d6) δ 13.28 (s, 1H), 8.43 (s, 1H), 7.58-7.26 (m, 5H), 5.85 (ddt, J=16.8, 10.3, 6.5 Hz, 1H), 5.10 (dq, J=17.2, 1.6 Hz, 1H), 5.01 (dq, J=10.2, 1.3 Hz, 1H), 4.76 (d, J=11.0 Hz, 1H), 4.65 (d, J=11.0 Hz, 1H), 2.55 (dd, J=9.6, 5.2 Hz, 2H), 2.23 (td, J=13.2, 10.0, 5.7 Hz, 2H), 1.27 (d, J=3.8 Hz, 18H) ppm. ESI-MS m z calc. 688.23315, found 689.0 (M+1)⁺; Retention time: 3.32 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=H₂O (0.05% CF₃CO₂H). Mobile phase B=CH₃CN (0.035% CF₃CO₂H). Flow rate=1.2 mL/min, injection volume=1.5 μL, and column temperature=60° C.

### C. Preparation of (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

### Step 1: tert-Butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate

Dissolved tert-butyl N-[2-[5-[(1R)-1-benzyloxy-1-(trifluoromethyl)pent-4-enyl]-1,3,4-oxadiazol-2-yl]-6-hydroxy-5-(trifluoromethyl)-3-pyridyl]-N-tert-butoxycarbonyl-carbamate (159.3 g, 231.3 mmol) and triphenylphosphine (72.9 g, 277.9 mmol) in toluene (1 L), then added (2S)-pent-4-en-2-ol (28.7 mL, 278.9 mmol). Heated this mixture to 45° C., then added DIAD (58.3 mL, 296.1 mmol) (exotherm) slowly over 40 min. For the next approximately 2 h, the mixture was cooled to room temperature. During this cooling period, after the first 10 minutes, triphenylphosphine (6.07 g, 23.14 mmol) was added. After a further 1 h, additional triphenylphosphine (3.04 g, 11.59 mmol) was added. After a further 23 min, DIAD (2.24 mL, 11.57 mmol) was added. After the ˜2 h cooling to room temperature period, the mixture was cooled to 15° C., and seed crystals of DIAD-triphenylphosphine oxide complex were added which caused precipitation to occur, then added 1000 mL heptane. Stored the mixture at −20° C. for 3 days. Filtered out and discarded the precipitate and concentrated the filtrate to give a red residue/oil. Dissolved the residue in 613 mL heptane at 45° C., then cooled to 0° C., seeded with DIAD-triphenylphosphine oxide complex, stirred at 0° C. for 30 min, then filtered the solution. The filtrate was concentrated to a smaller volume, then loaded onto a 1.5 kg silica gel column (column volume=2400 mL, flow rate=600 mL/min). Ran a gradient of 1% to 6% EtOAc in hexanes over 32 minutes (8 column volumes), then held at 6% EtOAc in hexanes until the product finished eluting which gave tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (163.5 g, 93%). ¹H NMR (400 MHz, Chloroform-d) δ 7.82 (s, 1H), 7.43-7.27 (m, 5H), 5.88-5.69 (m, 2H), 5.35 (h, J=6.2 Hz, 1H), 5.16-4.94 (m, 4H), 4.81 (d, J=10.7 Hz, 1H), 4.63 (d, J=10.7 Hz, 1H), 2.58-2.15 (m, 6H), 1.42 (s, 18H), 1.36 (d, J=6.2 Hz, 3H) ppm. ESI-MS m z calc. 756.2958, found 757.3 (M+1)⁺; Retention time: 4.0 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 2: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture)

The following reaction was run, split equally between two, 12 L reaction flasks run in parallel. Mechanical stirring was employed, and reactions were subjected to a constant nitrogen gas purge using a coarse porosity gas dispersion tube. To each flask was added tert-butyl N-[2-[5-[(1R)-1-benzyloxy-1-(trifluoromethyl)pent-4-enyl]-1,3,4-oxadiazol-2-yl]-6-[(1R)-1-methylbut-3-enoxy]-5-(trifluoromethyl)-3-pyridyl]-N-tert-butoxycarbonyl-carbamate (54 g, 71.36 mmol in each flask) dissolved in DCE (8 L in each flask) and both flasks were strongly purged with nitrogen at room temperature. Both flasks were heated to 62° C. and Grubbs 1^{st} Generation Catalyst (9 g, 10.94 mmol in each flask) was added to each reaction and stirred at 400 rpm while setting an internal temperature control to 75° C. with strong nitrogen purging (both reactions reached −75° C. after approximately 20 min). After 5 h 15 min, the internal temperature control was set to 45° C. After approximately 2 h, 2-sulfanylpyridine-3-carboxylic acid (11 g, 70.89 mmol in each flask) was added to each flask followed by triethylamine (10 mL, 71.75 mmol in each flask). On completion of addition, the nitrogen purge was turned off and both reaction flasks were stirred at 45° C. open to air overnight. The reactions were then removed from heat and 130 g of silica gel was added to each reaction and each was stirred at room temperature. After approximately 2 h, the green mixtures were combined and filtered over Celite then concentrated by rotary evaporation at 43° C. The obtained residue was dissolved in dichloromethane/heptane 1:1 (400 mL) and the formed orange solid was removed by filtration. The greenish mother liquor was evaporated to give 115.5 g of a green foam. Dissolved this material in 500 mL of 1:1 dichloromethane/hexanes then loaded onto a 3 kg silica gel column (column volume=4800 mL, flow rate=900 mL/min). Ran a gradient of 2% to 9% EtOAc in hexanes over 43 minutes (8 column volumes), then ran at 9% EtOAc until the product finished eluting giving 77.8 g of impure product. This material was co-evaporated with methanol (˜500 mL) then diluted with methanol (200 mL) to give 234.5 g of a methanolic solution, which was halved and each half was purified by reverse phase chromatography (3.8 kg C₁₈ column, column volume=3300 mL, flow rate=375 mL/min, loaded as solution in methanol). Ran the column at 55% acetonitrile for ˜5 minutes (0.5 column volumes), then at a gradient of 55% to 100% acetonitrile in water over ˜170 minutes (19-20 column volumes), then held at 100% acetonitrile until the product and impurities finished eluting. Clean product fractions from both columns were combined and concentrated by rotary evaporation then transferred with ethanol into 5 L flask, evaporated and carefully dried (becomes a foam) to give as a mixture of olefin isomers, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (55.5 g, 53%). ESI-MS m z calc. 728.26447, found 729.0 (M+1)⁺; Retention time: 3.82 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 3: tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,9,14,16-hexaen-17-yl]-N-tert-butoxycarbonyl-carbamate (E/Z mixture) (11.7 g, 16.06 mmol) was dissolved in stirring ethanol (230 mL) and cycled the flask 3 times vacuum/nitrogen and treated with 10% Pd/C (50% water wet, 2.2 g of 5% w/w, 1.034 mmol). The mixture was cycled 3 times between vacuum/nitrogen and 3 times between vacuum/hydrogen. The mixture was then stirred strongly under hydrogen (balloon) for 7.5 h. The catalyst was removed by filtration, replaced with fresh 10% Pd/C (50% water wet, 2.2 g of 5% w/w, 1.034 mmol) and stirred vigorously under hydrogen (balloon) overnight. Then, the catalyst was removed again by filtration, the filtrate evaporated and the residue (11.3 g, 1 g set aside) was dissolved in ethanol (230 mL) charged with fresh 10% Pd/C (50% water wet, 2.2 g of 5% w/w, 1.034 mmol) and stirred vigorously under hydrogen (balloon) for 6 h, recharged again with fresh 10% Pd/C (50% water wet, 2.2 g of 5% w/w, 1.034 mmol) and stirred vigorously under hydrogen (balloon) overnight. The catalyst was removed by filtration and the filtrate was evaporated (10 g of residue obtained). This crude material (10 g+1 g set aside above) was purified by silica gel chromatography (330 g column, liquid load in dichloromethane) with a linear gradient of 0% to 15% ethyl acetate in hexane until the product eluted followed by 15% to 100% ethyl acetate in hexane to giving, as a colorless foam, tert-butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (9.1 g, 78%). ESI-MS m z calc. 730.2801, found 731.0 (M+1)⁺; Retention time: 3.89 minutes. Final purity was determined by reversed phase UPLC using an Acquity UPLC BEH C₁₈ column (50×2.1 mm, 1.7 m particle) made by Waters (pn: 186002350), and a dual gradient run from 1-99% mobile phase B over 4.5 minutes. Mobile phase A=water (0.05% CF₃CO₂H). Mobile phase B=acetonitrile (0.035% CF₃CO₂H). Flow rate=1.2 mL/min, injection volume=1.5 μL, and column temperature=60° C.

### Step 4: (6R,12R)-17-amino-12-methyl-6,15-bis(trifluoromethyl)-13,19-dioxa-3,4,18-triazatricyclo[12.3.1.12,5]nonadeca-1(18),2,4,14,16-pentaen-6-ol

tert-Butyl N-[(6R,12R)-6-benzyloxy-12-methyl-6,15-bis(trifluoromethyl)-13,19-dioxa-3,4,18-triazatricyclo[12.3.1.12,5]nonadeca-1(18),2,4,14,16-pentaen-17-yl]-N-tert-butoxycarbonyl-carbamate (8.6 g, 11.77 mmol) was dissolved in ethanol (172 mL) then the flask was cycled 3 times between vacuum/nitrogen. Treated the mixture with 10% Pd/C (50% water wet, 1.8 g of 5% w/w, 0.8457 mmol) then cycled 3 times between vacuum/nitrogen and 3 times between vacuum/hydrogen and then stirred vigorously under hydrogen (balloon) at room temperature for 18 h. The mixture was cycled 3 times between vacuum/nitrogen, filtered over Celite washing with ethanol and then the filtrate was evaporated to give 7.3 g of tert-butyl N-tert-butoxycarbonyl-N-[(6R,12R)-6-hydroxy-12-methyl-6,15-bis(trifluoromethyl)-13,19-dioxa-3,4,18-triazatricyclo[12.3.1.12,5]nonadeca-1(18),2,4,14,16-pentaen-17-yl]carbamate an off-white solid. 1H NMR and MS confirmed the expected product. CFTR modulatory activity was confirmed using a standard Ussing Chamber Assay for CFTR potentiator activity.

### OTHER EMBODIMENTS

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following claims.

LENGTHY TABLES
The patent application contains a lengthy table section. A copy of the table is available in electronic form from the USPTO web site (https://seqdata.uspto.gov/?pageRequest=docDetail&DocID=US20240150377A1). An electronic copy of the table will also be available from the USPTO upon request and payment of the fee set forth in 37 CFR 1.19(b)(3).

1. A compound of Formula I: 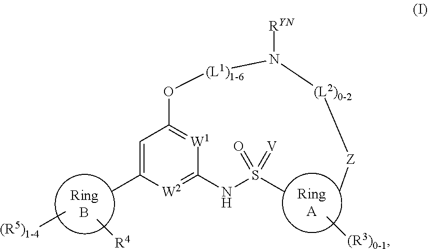
a tautomer thereof, deuterated derivative of the the compound or tautomer, or a pharmaceutically acceptable salt of any of the foregoing, wherein:
Ring A is selected from:
C₆-C₁₀ aryl,
C₃-C₁₀ cycloalkyl,
3- to 10-membered heterocyclyl, and
5- to 10-membered heteroaryl;
Ring B is selected from:
C₆-C₁₀ aryl,
C₃-C₁₀ cycloalkyl,
3- to 10-membered heterocyclyl, and
5- to 10-membered heteroaryl;
V is selected from O and NH;
W¹ is selected from N and CH;
W² is selected from N and CH, provided that at least one of W¹ nd W² is N;
Z is selected from O, NR^{ZN}, and C(R^{ZC})₂, provided that when L² is absent, Z is C(R^{ZC})₂;
each L¹ is independently selected from C(R^{L1})₂ and 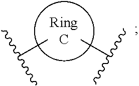
each L² is independently selected from C(R^{L2})₂;
Ring C is selected from C₆-C₁₀ aryl optionally substituted with 1-3 groups independently selected from:
halogen,
C₁-C₆ alkyl, and
N(RN)₂;
each R³ is independently selected from:
halogen,
C₁-C₆ alkyl,
C₁-C₆ alkoxy,
C₃-C₁₀ cycloalkyl,
C₆-C₁₀ aryl optionally substituted with 1-3 groups independently selected from C₁-C₆ alkyl, and
3- to 10-membered heterocyclyl;
R⁴ is selected from hydrogen and C₁-C₆ alkyl;
each R⁵ is independently selected from:
hydrogen,
halogen,
hydroxyl,
N(R^{N})₂,
—SO—Me,
—CH═C(R^{LC})₂, wherein both R^{LC} are taken together to form a C₃-C₁₀ cycloalkyl,
C₁-C₆ alkyl optionally substituted with 1-3 groups independently selected from:
hydroxyl,
C₁-C₆ alkoxy optionally substituted with 1-3 groups independently selected from C₁-C₆ alkoxy and C₆-C₁₀) aryl,
C₃-C₁₀) cycloalkyl,
—(O)₀₋₁—(C₆-C₁₀ aryl) optionally substituted with 1-3 groups independently selected from C₁-C₆ alkyl and C₁-C₆ alkoxy,
3- to 10-membered heterocyclyl, and
N(RN)₂,
C₁-C₆ alkoxy optionally substituted with 1-3 groups independently selected from:
halogen,
C₆-C₁₀ aryl, and
C₃-C₁₀ cycloalkyl optionally substituted with 1-3 groups independently selected from C₁-C₆ fluoroalkyl,
C₁-C₆ fluoroalkyl,
C₃-C₁₀ cycloalkyl,
C₆-C₁₀ aryl, and
3- to 10-membered heterocyclyl;
R^{VN} is selected from:
hydrogen,
C₁-C₉ alkyl optionally substituted with 1-3 groups independently selected from:
hydroxyl,
oxo,
cyano,
C₁-C₆ alkoxy optionally substituted with 1-3 groups independently selected from halogen and C₁-C₆ alkoxy,
N(R^{N})₂,
SO₂Me,
C₃-C₁₀ cycloalkyl optionally substituted with 1-3 groups independently selected from:
hydroxyl,
C₁-C₆ alkyl optionally substituted with 1-3 groups independently selected from hydroxyl, oxo, C₁-C₆ alkoxy, C₆-C₁₀ aryl, and N(RN)₂,
C₁-C₆ fluoroalkyl,
C₁-C₆ alkoxy,
COOH,
N(R^{N})₂,
C₆-C₁₀ aryl, and
3- to 10-membered heterocyclyl optionally substituted with 1-3 groups independently selected from oxo and C₁-C₆ alkyl,
C₆-C₁₀ aryl optionally substituted with 1-3 groups independently selected from:
halogen,
hydroxyl,
cyano,
SiMe₃,
SO₂Me,
SF₅,
N(R^{N})₂,
P(O)Me₂,
—(O)₀₋₁—(C₃-C₁₀ cycloalkyl) optionally substituted with 1-3 groups independently selected from C₁-C₆ fluoroalkyl,
C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
C₁-C₆ alkoxy optionally substituted with 1-3 groups independently selected from hydroxyl, oxo, N(R^{N})₂, and C₆-C₁₀ aryl,
C₁-C₆ fluoroalkyl,
3- to 10-membered heterocyclyl optionally substituted with 1-3 groups independently selected from C₁-C₆ alkyl,
(O)₀₋₁—(C₆-C₁₀ aryl), and
(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
3- to 10-membered heterocyclyl optionally substituted with 1-4 groups independently selected from:
hydroxyl,
oxo,
N(R^{N})₂,
C₁-C₆ alkyl optionally substituted with 1-3 groups independently selected from oxo and C₁-C₆ alkoxy,
C₁-C₆ alkoxy,
C₁-C₆ fluoroalkyl,
C₆-C₁₀ aryl optionally substituted with 1-3 groups independently selected from halogen, and
5- to 10-membered heteroaryl,
5- to 10-membered heteroaryl optionally substituted with 1-3 groups independently selected from:
hydroxyl,
cyano,
oxo,
halogen,
B(OH)₂,
N(R^{N})₂,
C₁-C₆ alkyl optionally substituted with 1-3 groups independently selected from hydroxyl, oxo, C₁-C₆ alkoxy (optionally substituted with 1-3 —SiMe3), 3- to 10-membered heterocyclyl, and N(R^{N})₂,
C₁-C₆ alkoxy optionally substituted with 1-3 groups independently selected from hydroxyl, oxo, halogen, C₁-C₆ alkoxy, N(R^{N})₂, 3- to 10-membered heterocyclyl (optionally substituted with 1-3 groups independently selected from C₁-C₆ alkyl), and C₃-C₁₀ cycloalkyl, C₁-C₆ fluoroalkyl,
—(O)₀₋₁—(C₃-C₁₀ cycloalkyl) optionally substituted with 1-3 groups independently selected from halogen, C₁-C₆ alkyl, and C₁-C₆ alkoxy,
—(O)₀₋₁—(C₆-C₁₀ aryl),
—(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, C₃-C₁₀ cycloalkyl, 5- to 10-membered heteroaryl (optionally substituted with 1-3 groups independently selected from C₁-C₆ alkyl), and 3- to 10-membered heterocyclyl (optionally substituted with 1-3 groups independently selected from C₁-C₆ fluoroalkyl), and 5- to 10-membered heteroaryl optionally substituted with 1-4 groups independently selected from C₁-C₆ alkyl and C₃-C₁₀ cycloalkyl, and
C₁-C₆ fluoroalkyl;
R^{ZN} is selected from:
hydrogen,
C₁-C₉ alkyl optionally substituted with 1-3 groups independently selected from:
hydroxyl,
oxo,
cyano,
C₁-C₆ alkoxy optionally substituted with 1-3 groups independently selected from halogen and C₁-C₆ alkoxy,
N(R^{N})₂,
SO₂Me,
C₃-C₁₀ cycloalkyl optionally substituted with 1-3 groups independently selected from:
hydroxyl,
C₁-C₆ alkyl optionally substituted with 1-3 groups independently selected from hydroxyl, oxo, C₁-C₆ alkoxy, C₆-Clo aryl, and N(R^{N})₂,
C₁-C₆ fluoroalkyl,
C₁-C₆ alkoxy,
COOH,
N(R^{N})₂,
C₆-C₁₀ aryl, and
3- to 10-membered heterocyclyl optionally substituted with 1-3 groups independently selected from oxo and C₁-C₆ alkyl,
C₆-C₁₀ aryl optionally substituted with 1-3 groups independently selected from:
halogen,
hydroxyl,
cyano,
SiMe₃,
SO₂Me,
SF5,
N(R^{N})₂,
P(O)Me₂,
—(O)₀₋₁—(C₃-C₁₀ cycloalkyl) optionally substituted with 1-3 groups independently selected from C₁-C₆ fluoroalkyl,
C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
C₁-C₆ alkoxy optionally substituted with 1-3 groups independently selected from hydroxyl, oxo, N(R^{N})₂, and C₆-C₁₀ aryl,
C₁-C₆ fluoroalkyl,
3- to 10-membered heterocyclyl optionally substituted with 1-3 groups independently selected from C₁-C₆ alkyl,
—(O)₀₋₁—(C₆-C₁₀ aryl), and
—(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
3- to 10-membered heterocyclyl optionally substituted with 1-4 groups independently selected from:
hydroxyl,
oxo,
N(R^{N})₂,
C₁-C₆ alkyl optionally substituted with 1-3 groups independently selected from oxo and C₁-C₆ alkoxy,
C₁-C₆ alkoxy,
C₁-C₆ fluoroalkyl,
C₆-C₁₀ aryl optionally substituted with 1-3 groups independently selected from halogen, and
5- to 10-membered heteroaryl,
5- to 10-membered heteroaryl optionally substituted with 1-3 groups independently selected from:
hydroxyl,
cyano,
oxo,
halogen,
B(OH)₂,
N(R^{N})₂,
C₁-C₆ alkyl optionally substituted with 1-3 groups independently selected from hydroxyl, oxo, C₁-C₆ alkoxy (optionally substituted with 1-3 —SiMe₃), and N(R^{N})₂,
C₁-C₆ alkoxy optionally substituted with 1-3 groups independently selected from hydroxyl, oxo, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
—(O)₀₋₁—(C₆-C₁₀ aryl),
—(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, 3- to 10-membered heterocyclyl (optionally substituted with 1-3 groups independently selected from C₁-C₆ fluoroalkyl), and
5- to 10-membered heteroaryl optionally substituted with 1-4 groups independently selected from C₁-C₆ alkyl and C₃-C₁₀ cycloalkyl,
C₁-C₆ fluoroalkyl,
C₃-C₁₀ cycloalkyl optionally substituted with 1-3 groups independently selected from:
hydroxyl,
oxo,
halogen,
cyano,
N(R^{N})₂,
C₁-C₆ alkyl optionally substituted with 1-3 groups independently selected from:
hydroxyl,
oxo,
N(R^{N})₂,
C₁-C₆ alkoxy, and
C₆-Cu) aryl,
C₁-C₆ alkoxy optionally substituted with 1-3 groups independently selected from halogen, oxo, C₆-C₁₀) aryl, and N(R^{N})₂,
halogen,
C₃-C₁₀) cycloalkyl,
3- to 10-member heterocyclyl optionally substituted with 1-3 groups independently selected from C₁-C₆ alkyl,
5- to 10-membered heteroaryl optionally substituted with 1-3 groups independently selected from:
hydroxyl,
cyano,
oxo,
halogen,
N(R^{N})₂,
C₁-C₆ alkyl optionally substituted with 1-3 groups independently selected from hydroxyl, oxo, C₁-C₆ alkoxy, and N(R^{N})₂,
C₁-C₆ alkoxy optionally substituted with 1-3 groups independently selected from hydroxyl, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
C₆-C₁₀ aryl,
3- to 10-membered heterocyclyl optionally substituted with 1-3 groups independently selected from C₁-C₆ alkyl,
C₆-C₁₀ aryl,
3- to 10-membered heterocyclyl optionally substituted with 1-3 groups independently selected from:
oxo,
C₁-C₆ alkyl optionally substituted with 1-3 groups independently selected from:
oxo,
hydroxyl,
N(R^{N})₂,
C₁-C₆ alkoxy optionally substituted with 1-3 groups independently selected from halogen and C₆-C₁₀ aryl, and
—(O)₀₋₁—(C₃-C₁₀ cycloalkyl),
C₁-C₆ fluoroalkyl,
C₃-C₁₀ cycloalkyl optionally substituted with 1-3 groups independently selected from halogen, and
3- to 10-membered heterocyclyl,
5- to 10-membered heteroaryl optionally substituted with 1-3 groups independently selected from:
halogen,
C₁-C₆ alkyl optionally substituted with 1-3 groups independently selected from oxo, C₁-C₆ alkoxy, and N(R^{N})₂, and
3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
R^{F.};
each R^{ZC} is independently selected from:
hydrogen,
C₁-C₆ alkyl optionally substituted with 1-3 groups independently selected from C₆-C₁₀ aryl (optionally substituted with 1-3 groups independently selected from C₁-C₆ alkyl),
C₆-C₁₀ aryl optionally substituted with 1-3 groups independently selected from C₁-C₆ alkyl, and
R^{F.};
or two R^{ZC} are taken together to form an oxo group;
each R^{L1} is independently selected from:
hydrogen,
N(R^{N})₂, provided that two N(R^{N})2 are not bonded to the same carbon,
C₁-C₉ alkyl optionally substituted with 1-3 groups independently selected from:
halogen,
hydroxyl,
oxo,
N(R^{N})₂,
C₁-C₆ alkoxy optionally substituted with 1-3 groups independently selected from C₆-C₁₀) aryl,
C₃-C₁₀ cycloalkyl optionally substituted with 1-3 groups independently selected from halogen, C₁-C₆ alkyl, and C₁-C₆ fluoroalkyl,
—O—(C₃-Cm cycloalkyl),
C₆-C₁₀ aryl optionally substituted with 1-3 groups independently selected from C₁-C₆ alkyl, and
3- to 10-membered heterocyclyl optionally substituted with 1-3 groups independently selected from C₁-C₆ alkyl (optionally substituted with 1-3 groups independently selected from hydroxyl and oxo),
C₃-C₁₀ cycloalkyl optionally substituted with 1-3 groups independently selected from C₁-C₆ alkyl,
C₆-C₁₀ aryl optionally substituted with 1-4 groups independently selected from:
halogen,
cyano,
SiMe₃,
POMe₂,
C₁-C₇ alkyl optionally substituted with 1-3 groups independently selected from:
hydroxyl,
oxo,
cyano,
SiMe₃,
N(R^{N})₂, and
C₃-C₁₀ cycloalkyl optionally substituted with 1-3 groups independently selected from C₁-C₆ fluoroalkyl,
C₁-C₆ alkoxy optionally substituted with 1-3 groups independently selected from:
C₃-C₁₀ cycloalkyl optionally substituted with 1-3 groups independently selected from C₁-C₆ fluoroalkyl, and
C₁-C₆ alkoxy,
C₁-C₆ fluoroalkyl,
C₃-C₁₀ cycloalkyl optionally substituted with 1-3 groups independently selected from C₁-C₆ alkyl and C₁-C₆ fluoroalkyl,
C₆-C₁) aryl,
3- to 10-membered heterocyclyl optionally substituted with 1-3 groups independently selected from C₁-C₆ alkyl, and
5- to 10-membered heteroaryl,
3- to 10-membered heterocyclyl optionally substituted with 1-3 groups independently selected from:
C₁-C₆ alkyl optionally substituted with 1-3 groups independently selected from:
oxo, and
C₁-C₆ alkoxy,
5- to 10-membered heteroaryl optionally substituted with 1-3 groups independently selected from:
C₁-C₆ alkyl optionally substituted with 1-3 groups independently selected from:
C₃-C₁₀ cycloalkyl optionally substituted with 1-3 groups independently selected from C₁-C₆ fluoroalkyl, and
C₆-C₁₀ aryl optionally substituted with 1-3 groups independently selected from C₁-C₆ alkyl, and
R^{F.};
or two R^{L1} on the same carbon atom are taken together to form an oxo group;
each R^{L2} is independently selected from hydrogen and R^{F.};
or two R^{L2} on the same carbon atom are taken together to form an oxo group;
each R^{N} is independently selected from:
hydrogen,
C₁-C₈ alkyl optionally substituted with 1-3 groups independently selected from:
oxo,
halogen,
hydroxyl,
NH₂,
NHMe,
NMe₂,
NHCOMe,
N(R^{N3})₂, wherein each R^{N3} is independently selected from C₁-C₆ alkyl (optionally substituted with 1-3 groups independently selected from oxo and C₁-C₆ alkoxy),
C₁-C₆ alkoxy optionally substituted with 1-3 groups independently selected from C₆-C₁₀ aryl, oxo, NMe₂, and NHMe,
—(O)₀₋₁—(C₃-C₁₀ cycloalkyl) optionally substituted with 1-3 groups independently selected from C₁-C₆ alkyl (optionally substituted with 1-3 groups independently selected from C₁-C₆ alkoxy), and C₁-C₆ alkoxy,
C₆-C₁₀ aryl optionally substituted with 1-3 groups independently selected from halogen and C₁-C₆ alkyl,
3- to 14-membered heterocyclyl optionally substituted with 1-4 groups independently selected from oxo and C₁-C₆ alkyl, and
5- to 14-membered heteroaryl optionally substituted with 1-4 groups independently selected from oxo and C₁-C₆ alkyl,
C₃-C₁₀ cycloalkyl optionally substituted with 1-3 groups independently selected from:
hydroxyl,
halogen,
NH₂,
NHMe,
C₁-C₆ alkoxy, and
C₁-C₆ alkyl optionally substituted with 1-3 groups independently selected from hydroxyl and C₁-C₆ alkoxy,
C₆-C₁₀ aryl,
5- to 10-membered heteroaryl optionally substituted with 1-3 groups independently selected from C₁-C₆ alkyl, and
3- to 10-membered heterocyclyl;
or two R^{N} on the same nitrogen atom are taken together with the nitrogen to which they are bonded to form a 3- to 10-membered heterocyclyl optionally substituted with 1-4 groups selected from:
hydroxyl,
halogen,
oxo,
cyano,
C₁-C₆ alkyl optionally substituted with 1-3 groups independently selected from oxo, hydroxyl, C₁-C₆ alkoxy, and N(RN2)2, wherein each R^{N2} is independently selected from hydrogen and C₁-C₆ alkyl,
C₁-C₆ alkoxy, and
C₁-C₆ fluoroalkyl;
or one R⁴and one R^{L1} are taken together to form a C₆-C₈ alkylene;
when R^{F} is present, two R^{F} taken together with the atoms to which they are bonded form a group selected from:
C₃-C₁₀ cycloalkyl optionally substituted with 1-3 groups independently selected from C₁-C₆ alkyl,
C₆-C₁₀ aryl optionally substituted with 1-3 groups independently selected from:
halogen,
C₁-C₆ alkyl,
N(R^{N})₂, and
3- to 10-membered heterocyclyl optionally substituted with 1-3 groups independently selected from hydroxyl,
3- to 11-membered heterocyclyl optionally substituted with 1-3 groups independently selected from:
oxo,
N(R^{N})₂,
C₁-C₉ alkyl optionally substituted with 1-4 groups independently selected from:
oxo,
halogen,
hydroxyl,
N(R^{N})₂,
—SO₂—(C₁-C₆ alkyl),
C₁-C₆ alkoxy optionally substituted with 1-3 groups independently selected from halogen and C₆-C₁₀ aryl,
C₆-C₁₀ aryl optionally substituted with 1-3 groups independently selected from hydroxyl, halogen, cyano, C₁-C₆ alkyl (optionally substituted with 1-3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
—(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
—O—(5- to 12-membered heteroaryl) optionally substituted with 1-3 groups independently selected from C₆-C₁₀ aryl (optionally substituted with 1-3 groups independently selected from halogen) and C₁-C₆ alkyl, and
5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), -O-(C₆-C₁₀ aryl), and C₃-C₁₀ cycloalkyl,
C₃-C₁₂ cycloalkyl optionally substituted with 1-4 groups independently selected from halogen, C₁-C₆ alkyl, and C₁-C₆ fluoroalkyl,
C₆-C₁₀ aryl,
3- to 10-membered heterocyclyl, and
5- to 10-membered heteroaryl optionally substituted with 1-3 groups independently selected from C₁-C₆ alkoxy and C₁-C₆ fluoroalkyl, and
5- to 12-membered heteroaryl optionally substituted with 1-3 groups independently selected from C₁-C₆ alkyl and C₁-C₆ fluoroalkyl.

2. A compound of Formula Ia: 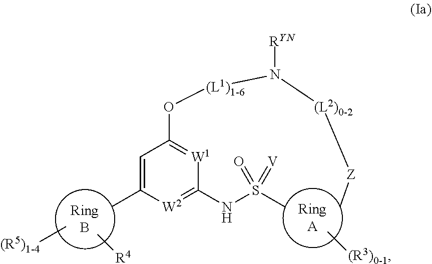
a tautomer thereof, a deuterated derivative of the compound or tautomer, or a pharmaceutically acceptable salt of any of the foregoing, wherein
Ring A is selected from:
C₆-C₁₀ aryl,
C₃-C₁₀ cycloalkyl,
3- to 10-membered heterocyclyl, and
5- to 10-membered heteroaryl;
Ring B is selected from:
C₆-C₁₀ aryl,
C₃-C₁₀ cycloalkyl,
3- to 10-membered heterocyclyl, and
5- to 10-membered heteroaryl;
V is selected from O and NH;
W¹ is selected from N and CH;
W2is selected from N and CH, provided that at least one of W¹ and W² is N;
Z is selected from O, NR^{ZN}, and C(R^{ZC})₂, provided that when L² is absent, Z is C(R^{ZC})₂; 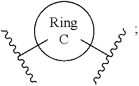
each L¹ is independently selected from C(R^{L1})₂ and
each L² is independently selected from C(R^{L2})₂;
Ring C is selected from C₆-C₁₀ aryl optionally substituted with 1-3 groups independently selected from:
halogen,
C₁-C₆ alkyl, and
N(R^{N})₂;
each R³ is independently selected from:
halogen,
C₁-C₆ alkyl,
C₁-C₆ alkoxy,
C₃-C₁₀ cycloalkyl,
C₆-C₁₀ aryl optionally substituted with 1-3 groups independently selected from C₁-C₆ alkyl, and
3- to 10-membered heterocyclyl;
R⁴ is selected from hydrogen and C₁-C₆ alkyl;
each R⁵ is independently selected from:
hydrogen,
halogen,
hydroxyl,
N(R^{N})₂,
—SO—Me,
—CH∇C(R^{LC})₂, wherein both R^{LC} are taken together to form a C₃-C₁₀ cycloalkyl,
C₁-C₆ alkyl optionally substituted with 1-3 groups independently selected from:
hydroxyl,
C₁-C₆ alkoxy optionally substituted with 1-3 groups independently selected from C₁-C₆ alkoxy and C₆-C₁₀) aryl,
C₃-C₁₀) cycloalkyl,
—(O)₀₋₁—(C₆-C₁₀ aryl) optionally substituted with 1-3 groups independently selected from C₁-C₆ alkyl and C₁-C₆ alkoxy,
3- to 10-membered heterocyclyl, and
N(R^{N})₂,
C₁-C₆ alkoxy optionally substituted with 1-3 groups independently selected from:
halogen,
C₆-C₁₀) aryl, and
C₃-C₁₀ cycloalkyl optionally substituted with 1-3 groups independently selected from C₁-C₆ fluoroalkyl,
C₁-C₆ fluoroalkyl,
C₃-C₁₀ cycloalkyl,
C₆-C₁₀ aryl, and
3- to 10-membered heterocyclyl;
R^{YN} is selected from:
hydrogen,
C₁-C₉ alkyl optionally substituted with 1-3 groups independently selected from:
hydroxyl,
oxo,
cyano,
C₁-C₆ alkoxy optionally substituted with 1-3 groups independently selected from halogen and C₁-C₆ alkoxy,
N(R^{N})₂,
SO₂Me,
C₃-C₁₀ cycloalkyl optionally substituted with 1-3 groups independently selected from:
hydroxyl,
C₁-C₆ alkyl optionally substituted with 1-3 groups independently selected from hydroxyl, oxo, C₁-C₆ alkoxy, C₆-C₁₀ aryl, and N(R^{N})₂,
C₁-C₆ fluoroalkyl,
C₁-C₆ alkoxy,
COOH,
N(R^{N})₂,
C₆-C₁₀ aryl, and
3- to 10-membered heterocyclyl optionally substituted with 1-3 groups independently selected from oxo and C₁-C₆ alkyl,
C₆-C₁₀ aryl optionally substituted with 1-3 groups independently selected from:
halogen,
hydroxyl,
cyano,
SiMe₃,
SO₂Me,
SF₅,
N(R^{N})₂,
P(O)Me₂,
—(O)₀₋₁—(C₃-C₁₀ cycloalkyl) optionally substituted with 1-3 groups independently selected from C₁-C₆ fluoroalkyl,
C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
C₁-C₆ alkoxy optionally substituted with 1-3 groups independently selected from hydroxyl, oxo, N(R^{N})₂, and C₆-C₁₀ aryl,
C₁-C₆ fluoroalkyl,
3- to 10-membered heterocyclyl optionally substituted with 1-3 groups independently selected from C₁-C₆ alkyl,
—(O)₀₋₁—(C₆-C₁₀ aryl), and
—(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
3- to 10-membered heterocyclyl optionally substituted with 1-4 groups independently selected from:
hydroxyl,
oxo,
N(R^{N})₂,
C₁-C₆ alkyl optionally substituted with 1-3 groups independently selected from oxo and C₁-C₆ alkoxy,
C₁-C₆ alkoxy,
C₁-C₆ fluoroalkyl,
C₆-C₁₀ aryl optionally substituted with 1-3 groups independently selected from halogen, and
5- to 10-membered heteroaryl, and
5- to 10-membered heteroaryl optionally substituted with 1-3 groups independently selected from:
hydroxyl,
cyano,
oxo,
halogen,
B(OH)₂,
N(R^{N})₂,
C₁-C₆ alkyl optionally substituted with 1-3 groups independently selected from hydroxyl, oxo, C₁-C₆ alkoxy (optionally substituted with 1-3 —SiMe₃), and N(R^{N})₂,
C₁-C₆ alkoxy optionally substituted with 1-3 groups independently selected from hydroxyl, oxo, halogen, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from halogen, C₁-C₆ alkyl, and C₁-C₆ alkoxy,
—(O)₀₋₁—(C₆-C₁₀ aryl),
—(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, and 3- to 10-membered heterocyclyl (optionally substituted with 1-3 groups independently selected from C₁-C₆ fluoroalkyl), and
5- to 10-membered heteroaryl optionally substituted with 1-4 groups independently selected from C₁-C₆ alkyl and C₃-C₁₀ cycloalkyl, and
C₁-C₆ fluoroalkyl;
R^{ZN} is selected from:
hydrogen,
C₁-C₉ alkyl optionally substituted with 1-3 groups independently selected from:
hydroxyl,
oxo,
cyano,
C₁-C₆ alkoxy optionally substituted with 1-3 groups independently selected from halogen and C₁-C₆ alkoxy,
N(R^{N})₂,
SO₂Me,
C₃-C₁₀ cycloalkyl optionally substituted with 1-3 groups independently selected from:
hydroxyl,
C₁-C₆ alkyl optionally substituted with 1-3 groups independently selected from hydroxyl, oxo, C₁-C₆ alkoxy, C₆-C₁₀ aryl, and N(R^{N})₂,
C₁-C₆ fluoroalkyl,
C₁-C₆ alkoxy,
COOH,
N(R^{N})₂,
C₆-C₁₀ aryl, and
3- to 10-membered heterocyclyl optionally substituted with 1-3 groups independently selected from oxo and C₁-C₆ alkyl,
C₆-C₁₀ aryl optionally substituted with 1-3 groups independently selected from:
halogen,
hydroxyl,
cyano,
SiMe₃,
SO₂Me,
SF₅,
N(R^{N})₂,
P(O)Me₂,
—(O)₀₋₁—(C₃-C₁₀ cycloalkyl) optionally substituted with 1-3 groups independently selected from C₁-C₆ fluoroalkyl,
C₁-C₆ alkyl optionally substituted with 1-3 groups independently selected from hydroxyl, oxo, C₁-C₆ alkoxy, 5- to 10-membered heteroaryl, SO₂Me, and N(R^{N})₂,
C₁-C₆ alkoxy optionally substituted with 1-3 groups independently selected from hydroxyl, oxo, N(R^{N})₂, and C₆-C₁₀ aryl,
C₁-C₆ fluoroalkyl,
3- to 10-membered heterocyclyl optionally substituted with 1-3 groups independently selected from C₁-C₆ alkyl,
—(O)₀₋₁—(C₆-C₁₀ aryl), and
—(O)₀₋₁—(5- to 10-heteroaryl) optionally substituted with hydroxyl, oxo, N(R^{N})₂, C₁-C₆ alkyl, C₁-C₆ alkoxy, C₁-C₆ fluoroalkyl, and C₃-C₁₀ cycloalkyl,
3- to 10-membered heterocyclyl optionally substituted with 1-4 groups independently selected from:
hydroxyl,
oxo,
N(R^{N})₂,
C₁-C₆ alkyl (optionally substituted with 1-3 groups independently selected from oxo and C₁-C₆ alkoxy),
C₁-C₆ alkoxy,
C₁-C₆ fluoroalkyl,
C₆-C₁₀ aryl optionally substituted with 1-3 groups independently selected from halogen, and
5- to 10-membered heteroaryl, and
5- to 10-membered heteroaryl optionally substituted with 1-3 groups independently selected from:
hydroxyl,
cyano,
oxo,
halogen,
B(OH)₂,
N(R^{N})₂,
C₁-C₆ alkyl optionally substituted with 1-3 groups independently selected from hydroxyl, oxo, C₁-C₆ alkoxy (optionally substituted with 1-3 -SiMe3), and N(R^{N})₂,
C₁-C₆ alkoxy optionally substituted with 1-3 groups independently selected from hydroxyl, oxo, C₁-C₆ alkoxy, N(R^{N})₂, and C₃-C₁₀ cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
—(O)₀₋₁—(C₆-C₁₀ aryl),
—(O)₀₋₁—(3- to 10-membered heterocyclyl) optionally substituted with 1-4 groups independently selected from hydroxyl, oxo, halogen, cyano, N(R^{N})₂, C₁-C₆ alkyl (optionally substituted with 1-3 groups independently selected from hydroxyl, oxo, N(R^{N})₂, and C₁-C₆ alkoxy), C₁-C₆ alkoxy, C₁-C₆ fluoroalkyl, and 3- to 10-membered heterocyclyl (optionally substituted with 1-3 groups independently selected from C₁-C₆ fluoroalkyl), and
5- to 10-membered heteroaryl optionally substituted with 1-4 groups independently selected from C₁-C₆ alkyl and C₃-C₁₀ cycloalkyl,
C₁-C₆ fluoroalkyl,
C₃-C₁₀ cycloalkyl optionally substituted with 1-3 groups independently selected from:
hydroxyl,
oxo,
halogen,
cyano,
N(R^{N})₂,
C₁-C₆ alkyl optionally substituted with 1-3 groups independently selected from:
hydroxyl,
oxo,
N(R^{N})₂,
C₁-C₆ alkoxy, and
C₆-C₁₀ aryl,
C₁-C₆ alkoxy optionally substituted with 1-3 groups independently selected from halogen, oxo, C₆-Clo aryl, and N(R^{N})₂,
halogen,
C₃-C₁₀ cycloalkyl,
3- to 10-memember heterocyclyl optionally substituted with 1-3 groups independently selected from C₁-C₆ alkyl,
5- to 10-membered heteroaryl optionally substituted with 1-3 groups independently selected from:
hydroxyl,
cyano,
oxo,
halogen,
N(R^{N})₂,
C₁-C₆ alkyl optionally substituted with 1-3 groups independently selected from hydroxyl, oxo, C₁-C₆ alkoxy, and N(R^{N})₂,
C₁-C₆ alkoxy optionally substituted with 1-3 groups independently selected from hydroxyl, C₁-C₆ alkoxy, N(R^{N})₂, and C₃-Clo cycloalkyl,
C₁-C₆ fluoroalkyl,
—(O)₀₋₁—(C₃-C₁₀ cycloalkyl) optionally substituted with 1-3 groups independently selected from C₁-C₆ alkyl,
C₆-C₁₀ aryl, and
3- to 10-membered heterocyclyl optionally substituted with 1-3 groups independently selected from C₁-C₆ alkyl,
C₆-C₁₀ aryl,
3- to 10-membered heterocyclyl optionally substituted with 1-3 groups independently selected from:
oxo,
C₁-C₆ alkyl optionally substituted with 1-3 groups independently selected from:
oxo,
hydroxyl,
N(R^{N})₂,
C₁-C₆ alkoxy optionally substituted with 1-3 groups independently selected from halogen and C₆-C₁₀ aryl, and
—(O)₀₋₁—(C₃-Cm cycloalkyl),
C₁-C₆ fluoroalkyl,
C₃-C₁₀ cycloalkyl optionally substituted with 1-3 groups independently selected from halogen, and
3- to 10-membered heterocyclyl,
5- to 10-membered heteroaryl optionally substituted with 1-3 groups independently selected from:
halogen,
C₁-C₆ alkyl optionally substituted with 1-3 groups independently selected from oxo, C₁-C₆ alkoxy, and N(R^{N})₂, and
3- to 10-membered heterocyclyl optionally substituted with 1-3 groups independently selected from C₁-C₆ alkyl (optionally substituted with 1-3 groups selected from oxo, C₁-C₆ alkoxy, and C₆-C₁₀ aryl), and
R^{F};
each R^{ZC} is independently selected from:
hydrogen,
C₁-C₆ alkyl optionally substituted with 1-3 groups independently selected from C₆-C₁₀ aryl (optionally substituted with 1-3 groups independently selected from C₁-C₆ alkyl), and
C₆-C₁₀ aryl optionally substituted with 1-3 groups independently selected from C₁-C₆ alkyl, and
R^{F};
or two R^{ZC} are taken together to form an oxo group;
each R^{L1} is independently selected from:
hydrogen,
N(R^{N})₂, provided that two N(R^{N})₂ are not bonded to the same carbon,
C₁-C₉ alkyl optionally substituted with 1-3 groups independently selected from:
halogen,
hydroxyl,
oxo,
N(R^{N})₂,
C₁-C₆ alkoxy optionally substituted with 1-3 groups independently selected from C₆-C₁₀) aryl,
C₃-C₁₀ cycloalkyl optionally substituted with 1-3 groups independently selected from halogen and C₁-C₆ fluoroalkyl,
C₆-C₁₀ aryl optionally substituted with 1-3 groups independently selected from C₁-C₆ alkyl, and
3- to 10-membered heterocyclyl optionally substituted with 1-3 groups independently selected from C₁-C₆ alkyl (optionally substituted with 1-3 groups independently selected from hydroxyl and oxo),
C₃-C₁₀ cycloalkyl,
C₆-C₁₀ aryl optionally substituted with 1-4 groups independently selected from:
halogen,
cyano,
SiMe₃,
POMe₂,
C₁-C₇ alkyl optionally substituted with 1-3 groups independently selected from:
hydroxyl,
oxo,
cyano,
SiMe₃,
N(R^{N})₂, and
C₃-C₁₀ cycloalkyl optionally substituted with 1-3 groups independently selected from C₁-C₆ fluoroalkyl,
C₁-C₆ alkoxy optionally substituted with 1-3 groups independently selected from:
C₃-C₁₀ cycloalkyl optionally substituted with 1-3 groups independently selected from C₁-C₆ fluoroalkyl, and
C₁-C₆ alkoxy,
C₁-C₆ fluoroalkyl,
C₃-C₁₀ cycloalkyl optionally substituted with 1-3 groups independently selected from C₁-C₆ alkyl and C₁-C₆ fluoroalkyl,
C₆-C₁) aryl,
3- to 10-membered heterocyclyl optionally substituted with 1-3 groups independently selected from C₁-C₆ alkyl, and
5- to 10-membered heteroaryl,
3- to 10-membered heterocyclyl optionally substituted with 1-3 groups independently selected from:
C₁-C₆ alkyl optionally substituted with 1-3 groups independently selected from:
oxo, and
C₁-C₆ alkoxy,
5- to 10-membered heteroaryl optionally substituted with 1-3 groups independently selected from:
C₁-C₆ alkyl optionally substituted with 1-3 groups independently selected from:
C₃-C₁₀ cycloalkyl optionally substituted with 1-3 groups independently selected from C₁-C₆ fluoroalkyl, and
C₆-C₁₀ aryl optionally substituted with 1-3 groups independently selected from C₁-C₆ alkyl, and
R^{F};
or two R^{L1} on the same carbon atom are taken together to form an oxo group;
each R^{L2} is independently selected from hydrogen and RF;
or two R^{L2} on the same carbon atom are taken together to form an oxo group;
each R^{N} is independently selected from:
hydrogen,
C₁-Cs alkyl optionally substituted with 1-3 groups independently selected from:
oxo,
halogen,
hydroxyl,
NH₂,
NHMe,
NMe₂,
NHCOMe,
C₁-C₆ alkoxy optionally substituted with 1-3 groups independently selected from C₆-Cm aryl,
—(O)₀₋₁—(C₃-Cm cycloalkyl),
C₆-C₁₀ aryl optionally substituted with 1-3 groups independently selected from halogen and C₁-C₆ alkyl, and
3- to 14-membered heterocyclyl optionally substituted with 1-4 groups independently selected from oxo and C₁-C₆ alkyl, and
5- to 14-membered heteroaryl optionally substituted with 1-4 groups independently selected from oxo and C₁-C₆ alkyl,
C₃-C₁₀ cycloalkyl optionally substituted with 1-3 groups independently selected from:
hydroxyl,
NH₂,
NHMe, and
C₁-C₆ alkyl optionally substituted with 1-3 groups independently selected from hydroxyl,
C₆-C₁₀ aryl, and
3- to 10-membered heterocyclyl;
or two R^{N} on the same nitrogen atom are taken together with the nitrogen to which they are bonded to form a 3- to 10-membered heterocyclyl optionally substituted with 1-4 groups selected from:
hydroxyl,
halogen,
oxo,
cyano,
C₁-C₆ alkyl optionally substituted with 1-3 groups independently selected from oxo, hydroxyl, C₁-C₆ alkoxy, and N(R^{N2})₂, wherein each R^{N2} is independently selected from hydrogen and C₁-C₆ alkyl,
C₁-C₆ alkoxy, and
C₁-C₆ fluoroalkyl;
or one R⁴ and one R^{L1} are taken together to form a C₆-C₈ alkylene;
when R^{F} is present, two R^{F} taken together with the atoms to which they are bonded form a group selected from:
C₃-C₁₀ cycloalkyl optionally substituted with 1-3 groups independently selected from C₁-C₆ alkyl,
C₆-C₁₀ aryl optionally substituted with 1-3 groups independently selected from:
halogen,
C₁-C₆ alkyl,
N(R^{N})₂, and
3- to 10-membered heterocyclyl optionally substituted with 1-3 groups independently selected from hydroxyl,
3- to 11-membered heterocyclyl optionally substituted with 1-3 groups independently selected from:
oxo,
N(R^{N})₂,
C₁-C₉ alkyl optionally substituted with 1-4 groups independently selected from:
oxo,
halogen,
hydroxyl,
N(R^{N})₂,
—SO₂—(C₁-C₆ alkyl),
C₁-C₆ alkoxy optionally substituted with 1-3 groups independently selected from halogen and C₆-C₁₀ aryl,
C₆-C₁₀ aryl optionally substituted with 1-3 groups independently selected from hydroxyl, halogen, cyano, C₁-C₆ alkyl (optionally substituted with 1- 3 groups independently selected from oxo and C₁-C₆ alkoxy), C₁-C₆ alkoxy (optionally substituted with 1-3 groups independently selected from C₆-C₁₀ aryl), —(O)₀₋₁—(C₁-C₆ fluoroalkyl), and C₆-C₁₀ aryl (optionally substituted with 1-3 groups independently selected from C₁-C₆ alkoxy),
—(O)₀₋₁—(C₃-C₁₀ cycloalkyl) optionally substituted with 1-4 groups independently selected from hydroxyl, halogen, N(R^{N})₂, C₁-C₆ alkyl (optionally substituted with 1-3 groups independently selected from oxo, hydroxyl, and C₁-C₆ alkoxy), C₁-C₆ fluoroalkyl, and C₆-C₁₀ aryl,
3- to 10-membered heterocyclyl optionally substituted with 1-3 groups independently selected from oxo, C₁-C₆ alkyl (optionally substituted with 1-3 groups independently selected from C₆-C₁₀ aryl (optionally substituted with 1-3 groups independently selected from halogens)), C₁-C₆ alkoxy, C₃-C₁₀ cycloalkyl, and R^{N},
—O—(5- to 12-membered heteroaryl) optionally substituted with 1-3 groups independently selected from C₆-C₁₀ aryl (optionally substituted with 1-3 groups independently selected from halogen) and C₁-C₆ alkyl, and
5- to 10-membered heteroaryl optionally substituted with 1-3 groups independently selected from hydroxyl, oxo, N(R^{N})₂, C₁-C₆ alkyl (optionally substituted with 1-3 groups independently selected from cyano), C₁-C₆ alkoxy, —(O)₀₋₁—(C₁-C₆ fluoroalkyl), -O-(C₆-C₁₀ aryl), and C₃-C₁₀ cycloalkyl,
C₃-C₁₂ cycloalkyl optionally substituted with 1-4 groups independently selected from halogen, C₁-C₆ alkyl, and C₁-C₆ fluoroalkyl,
C₆-C₁₀ aryl,
3- to 10-membered heterocyclyl, and
5- to 10-membered heteroaryl optionally substituted with 1-3 groups independently selected from C₁-C₆ alkoxy and C₁-C₆ fluoroalkyl, and
5- to 12-membered heteroaryl optionally substituted with 1-3 groups independently selected from C₁-C₆ alkyl and C₁-C₆ fluoroalkyl.

3. The compound, tautomer, deuterated derivative, or pharmaceutically acceptable salt according to claim 2, selected from compounds of Formula Ib: 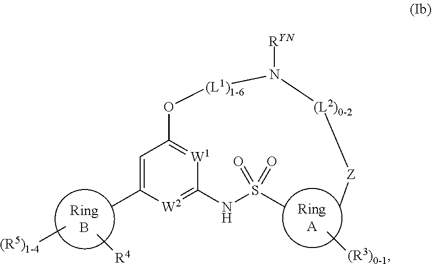
tautomers thereof, deuterated derivatives of the compounds and tautomers, and pharmaceutically acceptable salts of the compounds, tautomers, and deuterated derivatives , wherein Ring A, Ring B, W¹, W², Z, L¹, L², R³, R⁴, R⁵, and R^{YN} are defined as according to claim 2.

4. The compound, tautomer, deuterated derivative, or pharmaceutically acceptable salt according to claim 2, selected from compounds of Formula IIa: 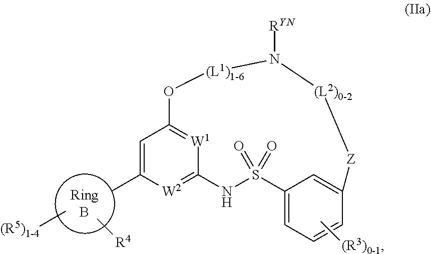
tautomers thereof, deuterated derivatives of the compounds and tautomers, and pharmaceutically acceptable salts of the compounds, tautomers, and deuterated derivatives a tautomer thereof, a deuterated derivative of the compound or tautomer, or a pharmaceutically acceptable salt of any of the foregoing, wherein Ring B, W¹, W², Z, L¹, L², R³, R⁴, R⁵, and R^{YN} are defined as according to claim 2.

5. The compound, tautomer, deuterated derivative, or pharmaceutically acceptable salt according to claim 2, selected from compounds of Formula IIb. 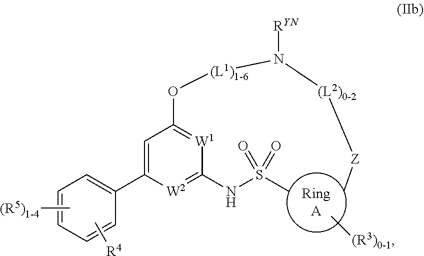
tautomers thereof, deuterated derivatives of the compounds and tautomers, and pharmaceutically acceptable salts of the compounds, tautomers, and deuterated derivatives, wherein Ring A, W¹, W², Z, L¹, L², R², R⁴, R⁵, and R^{YN} are defined as according to claim 2.

6. The compound, tautomer, deuterated derivative, or pharmaceutically acceptable salt according to claim 2, selected from compounds of Formula III: 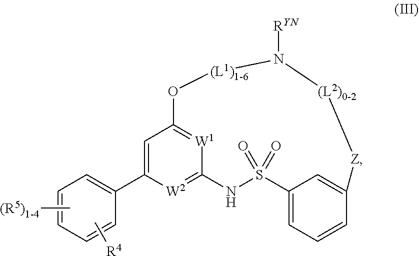
tautomers thereof, deuterated derivatives of the compounds and tautomers, and pharmaceutically acceptable salts of the compounds, tautomers, and deuterated derivatives, wherein Z, L¹, L², R², R⁴, R⁵, and R^{YN} are defined as according to claim 2.

7. The compound, tautomer, deuterated derivative, or pharmaceutically acceptable salt according to claim 2, selected from compounds of Formula IV: 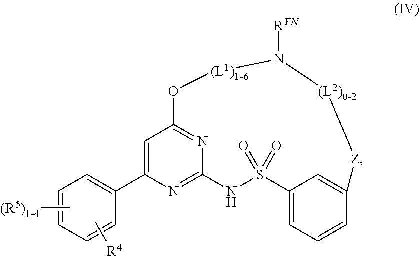
tautomers thereof, deuterated derivatives of the compounds and tautomers, and pharmaceutically acceptable salts of the compounds, tautomers, and deuterated derivatives, wherein Z, L¹, L², R⁴, R⁵, and R^{YN} are defined as according to claim 2.

8. The compound, tautomer, deuterated derivative, or pharmaceutically acceptable salt according to claim 2, selected from compounds A- compound of Formula V: 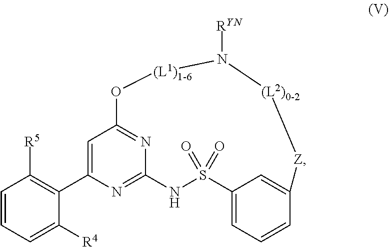
tautomers thereof, deuterated derivatives of the compounds and tautomers, and pharmaceutically acceptable salts of the compounds, tautomers, and deuterated derivatives, wherein Z, L¹, L², R⁴, R⁵, and R^{YN} are defined as according to claim 2.

9. The compound, tautomer, deuterated derivative, or pharmaceutically acceptable salt according to claim 2, selected from compounds of Formula VI: 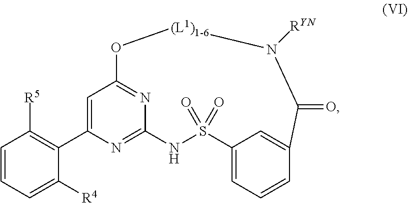
tautomers thereof, deuterated derivatives of the compounds and tautomers, and pharmaceutically acceptable salts of the compounds, tautomers, and deuterated derivatives, wherein L¹, R⁴, R⁵, and R^{YN} are defined as according to claim 2.

10. (canceled)

11. The compound, tautomer, deuterated derivative, or pharmaceutically acceptable salt according claim 1, selected from Compounds 1-1193, Compounds 1194-1294, tautomers thereof, deuterated derivatives of the compounds and tautomers, and pharmaceutically acceptable salts of the compounds, tautomers, and deuterated derivatives.

12. A pharmaceutical composition comprising the compound, tautomer, deuterated derivative, or pharmaceutically acceptable salt according to claim 1, and a pharmaceutically acceptable carrier.

13. The pharmaceutical composition of claim 12, further comprising one or more additional therapeutic agents.

14. The pharmaceutical composition of claim 13, wherein the one or more additional therapeutic agents are selected from tezacaftor, ivacaftor, deutivacaftor, lumacaftor, (6R,12R)-17-amino-12-methyl-6,15-bis(trifluoromethyl)-13,19-dioxa-3,4,18- triazatricyclo[12.3.1.12,5]nonadeca-1(18),2,4,14,16-pentaen-6-ol, and pharmaceutically acceptable salts thereof.

15. The pharmaceutical composition of claim 14, wherein the composition comprises tezacaftor and ivacaftor.

16. The pharmaceutical composition of claim 14, wherein the composition comprises tezacaftor and deutivacaftor.

17. The pharmaceutical composition of claim 14, wherein the composition comprises tezacaftor and (6R,12R)-17-amino-12-methyl-6,15-bis(trifluoromethyl)-13,19- dioxa-3,4,18-triazatricyclo[12.3.1.12,5]nonadeca-1(18),2,4,14,16-pentaen-6-ol.

18. A method of treating cystic fibrosis comprising administering to a patient in need thereof the compound, tautomer, deuterated derivative, or pharmaceutically acceptable salt according claim 1.

19. The method of claim 18, further comprising administering to the patient one or more additional therapeutic agents prior to, concurrent with, or subsequent to the compound, tautomer, deuterated derivative, or pharmaceutically acceptable salt according to claim 1.

20. The method of claim 19, wherein the one or more additional therapeutic agents is a compound selected from tezacaftor, ivacaftor, deutivacaftor, lumacaftor, (6R,12R)- 17-amino-12-methyl-6,15-bis(trifluoromethyl)-13,19-dioxa-3,4,18-triazatricyclo [12.3.1.12,5]nonadeca-1(18),2,4,14,16-pentaen-6-ol, and pharmaceutically acceptable salts thereof.

21. The method of claim 20, wherein the one or more additional therapeutic agents are tezacaftor and ivacaftor.

22. The method of claim 20, wherein the one or more additional therapeutic agents are tezacaftor and deutivacaftor.

23. The method of claim 20, wherein the one or more additional therapeutic agents are tezacaftor and (6R,12R)-17-amino-12-methyl-6,15-bis(trifluoromethyl)-13,19-dioxa- 3,4,18-triazatricyclo[12.3.1.12,5]nonadeca-1(18),2,4,14,16-pentaen-6-ol.

24-25. (canceled)
